SEQUENCE LISTING (534 sequences)

SEQ ID NO: 1 - DNA, 22 bp, Artificial Sequence
  Description of Artificial Sequence Note = Synthetic Construct
ctaacatacaatgctgctaggc

SEQ ID NO: 2 - DNA, 21 bp, Artificial Sequence
  Description of Artificial Sequence Note = Synthetic Construct
caatctttgcatctcggaagt

SEQ ID NO: 3 - DNA, 19 bp, Artificial Sequence
  Description of Artificial Sequence Note = Synthetic Construct
agaactaggtggtgtctac

SEQ ID NO: 4 - DNA, 19 bp, Artificial Sequence
  Description of Artificial Sequence Note = Synthetic Construct
gattttccctaacaggtgc

SEQ ID NO: 5 - DNA, 22 bp, Artificial Sequence
  Description of Artificial Sequence Note = Synthetic Construct
catgtgttcaaagtcaaggata

SEQ ID NO: 6 - DNA, 17 bp, Artificial Sequence
  Description of Artificial Sequence Note = Synthetic Construct
tgcttgtggtaatcggt

SEQ ID NO: 7 - DNA, 17 bp, Artificial Sequence
  Description of Artificial Sequence Note = Synthetic Construct
gtgttcggttatggagc

SEQ ID NO: 8 - DNA, 22 bp, Artificial Sequence
  Description of Artificial Sequence Note = Synthetic Construct
ggtatcatcttctttgttggga

SEQ ID NO: 9 - DNA, 16 bp, Artificial Sequence
  Description of Artificial Sequence Note = Synthetic Construct
cgcagggagcaagagt

SEQ ID NO: 10 - DNA, 20 bp, Artificial Sequence
  Description of Artificial Sequence Note = Synthetic Construct
cttcaaaaccaacaaggcag

SEQ ID NO: 11 - DNA, 21 bp, Artificial Sequence
  Description of Artificial Sequence Note = Synthetic Construct
agctttgtataagtttcgtgt

SEQ ID NO: 12 - DNA, 17 bp, Artificial Sequence
  Description of Artificial Sequence Note = Synthetic Construct
ccagccttcctcatctc

SEQ ID NO: 13 - DNA, 21 bp, Artificial Sequence
  Description of Artificial Sequence Note = Synthetic Construct
atgacatcaagatacctgtag

SEQ ID NO: 14 - DNA, 17 bp, Artificial Sequence
  Description of Artificial Sequence Note = Synthetic Construct
gacccattgctccttcg

SEQ ID NO: 15 - DNA, 17 bp, Artificial Sequence
  Description of Artificial Sequence Note = Synthetic Construct
gcaccacaaggtgtacg

SEQ ID NO: 16 - DNA, 17 bp, Artificial Sequence
  Description of Artificial Sequence Note = Synthetic Construct
gcccgacatcctcaaag

SEQ ID NO: 17 - DNA, 20 bp, Artificial Sequence
  Description of Artificial Sequence Note = Synthetic Construct
gaatgtggagactgaagcaa

SEQ ID NO: 18 - DNA, 24 bp, Artificial Sequence
  Description of Artificial Sequence Note = Synthetic Construct
caaatggaggatcattctgatagg

SEQ ID NO: 19 - DNA, 19 bp, Artificial Sequence
  Description of Artificial Sequence Note = Synthetic Construct
aggacagcatagacgacac

SEQ ID NO: 20 - DNA, 19 bp, Artificial Sequence
  Description of Artificial Sequence Note = Synthetic Construct
aggattctgcacagagcca

SEQ ID NO: 21 - DNA, 18 bp, Artificial Sequence
  Description of Artificial Sequence Note = Synthetic Construct
tcctgtgtggacctggat

SEQ ID NO: 22 - DNA, 17 bp, Artificial Sequence
  Description of Artificial Sequence Note = Synthetic Construct
tgccgtcgcttgatgag

SEQ ID NO: 23 - DNA, 20 bp, Artificial Sequence
  Description of Artificial Sequence Note = Synthetic Construct
catgatcaggtccaccttct

SEQ ID NO: 24 - DNA, 20 bp, Artificial Sequence
  Description of Artificial Sequence Note = Synthetic Construct
agcagcatgtcgaagatctc

SEQ ID NO: 25 - DNA, 18 bp, Artificial Sequence
  Description of Artificial Sequence Note = Synthetic Construct
ccctttctcctgggaaac

SEQ ID NO: 26 - DNA, 17 bp, Artificial Sequence
  Description of Artificial Sequence Note = Synthetic Construct
gctttggacagtggtct

SEQ ID NO: 27 - DNA, 20 bp, Artificial Sequence
  Description of Artificial Sequence Note = Synthetic Construct
gttaggaactgtgaagatgg

SEQ ID NO: 28 - DNA, 16 bp, Artificial Sequence
  Description of Artificial Sequence Note = Synthetic Construct
gccgctcgtagtcatg

SEQ ID NO: 29 - DNA, 20 bp, Artificial Sequence
  Description of Artificial Sequence Note = Synthetic Construct
agccattttgtcctgttttc

SEQ ID NO: 30 - DNA, 18 bp, Artificial Sequence
  Description of Artificial Sequence Note = Synthetic Construct
ccttcctcttcgttcact

SEQ ID NO: 31 - DNA, 16 bp, Artificial Sequence
  Description of Artificial Sequence Note = Synthetic Construct
agggaccgtgactcaa

SEQ ID NO: 32 - DNA, 18 bp, Artificial Sequence
  Description of Artificial Sequence Note = Synthetic Construct
aaacagaggatacctggc

SEQ ID NO: 33 - DNA, 19 bp, Artificial Sequence
  Description of Artificial Sequence Note = Synthetic Construct
aactgtcagaccaccacaa

SEQ ID NO: 34 - DNA, 20 bp, Artificial Sequence
  Description of Artificial Sequence Note = Synthetic Construct
gaagtcctccagtgagtcat

SEQ ID NO: 35 - DNA, 19 bp, Artificial Sequence
  Description of Artificial Sequence Note = Synthetic Construct
tcgatgcacacactggtat

SEQ ID NO: 36 - DNA, 19 bp, Artificial Sequence
  Description of Artificial Sequence Note = Synthetic Construct
ttcacatctgccacgtact

SEQ ID NO: 37 - DNA, 16 bp, Artificial Sequence
  Description of Artificial Sequence Note = Synthetic Construct
gggctctatgggaagg

SEQ ID NO: 38 - DNA, 16 bp, Artificial Sequence
  Description of Artificial Sequence Note = Synthetic Construct
gttctgggacagcagg

SEQ ID NO: 39 - DNA, 18 bp, Artificial Sequence
  Description of Artificial Sequence Note = Synthetic Construct
tggggctaagtggactat

SEQ ID NO: 40 - DNA, 17 bp, Artificial Sequence
  Description of Artificial Sequence Note = Synthetic Construct
tgccttctgagggtcaa

SEQ ID NO: 41 - DNA, 17 bp, Artificial Sequence
  Description of Artificial Sequence Note = Synthetic Construct
gcccttctacaaccctg

SEQ ID NO: 42 - DNA, 17 bp, Artificial Sequence
  Description of Artificial Sequence Note = Synthetic Construct
gctccaagtgcaagttc

SEQ ID NO: 43 - DNA, 17 bp, Artificial Sequence
  Description of Artificial Sequence Note = Synthetic Construct
cacgcacctgctgaaat

SEQ ID NO: 44 - DNA, 19 bp, Artificial Sequence
  Description of Artificial Sequence Note = Synthetic Construct
tctaccacgggcttctgtc

SEQ ID NO: 45 - DNA, 17 bp, Artificial Sequence
  Description of Artificial Sequence Note = Synthetic Construct
gagattgccacctaccg

SEQ ID NO: 46 - DNA, 17 bp, Artificial Sequence
  Description of Artificial Sequence Note = Synthetic Construct
gaggagatgaccttgcc

SEQ ID NO: 47 - DNA, 17 bp, Artificial Sequence
  Description of Artificial Sequence Note = Synthetic Construct
ggagaaggagttggacc

SEQ ID NO: 48 - DNA, 17 bp, Artificial Sequence
  Description of Artificial Sequence Note = Synthetic Construct
ccactgctgctggagta

SEQ ID NO: 49 - DNA, 17 bp, Artificial Sequence
  Description of Artificial Sequence Note = Synthetic Construct
acagcactccagccaaa

SEQ ID NO: 50 - DNA, 19 bp, Artificial Sequence
  Description of Artificial Sequence Note = Synthetic Construct
ctggtatgagcgtccaaac

SEQ ID NO: 51 - DNA, 19 bp, Artificial Sequence
  Description of Artificial Sequence Note = Synthetic Construct
agctcacagcgtttctatc

SEQ ID NO: 52 - DNA, 21 bp, Artificial Sequence
  Description of Artificial Sequence Note = Synthetic Construct
tgtgcagcaataacttcagac

SEQ ID NO: 53 - DNA, 18 bp, Artificial Sequence
  Description of Artificial Sequence Note = Synthetic Construct
cgtgccgactattgacat

SEQ ID NO: 54 - DNA, 17 bp, Artificial Sequence
  Description of Artificial Sequence Note = Synthetic Construct
gtagcggacgacaaagg

SEQ ID NO: 55 - DNA, 22 bp, Artificial Sequence
  Description of Artificial Sequence Note = Synthetic Construct
tcaaagattccaacggtcatag

SEQ ID NO: 56 - DNA, 22 bp, Artificial Sequence
  Description of Artificial Sequence Note = Synthetic Construct
tctcaagttccacttccagtag

SEQ ID NO: 57 - DNA, 18 bp, Artificial Sequence
  Description of Artificial Sequence Note = Synthetic Construct
atgtcagtgagcaagtcc

SEQ ID NO: 58 - DNA, 20 bp, Artificial Sequence
  Description of Artificial Sequence Note = Synthetic Construct
gctggttaatgtctgtcagt

SEQ ID NO: 59 - DNA, 19 bp, Artificial Sequence
  Description of Artificial Sequence Note = Synthetic Construct
gctgagatatggcaagtcc

SEQ ID NO: 60 - DNA, 19 bp, Artificial Sequence
  Description of Artificial Sequence Note = Synthetic Construct
ctcctaatcgcaaaagagc

SEQ ID NO: 61 - DNA, 20 bp, Artificial Sequence
  Description of Artificial Sequence Note = Synthetic Construct
caaaaatctccagccctaca

SEQ ID NO: 62 - DNA, 21 bp, Artificial Sequence
  Description of Artificial Sequence Note = Synthetic Construct
taaccatcctttccagcatac

SEQ ID NO: 63 - DNA, 17 bp, Artificial Sequence
  Description of Artificial Sequence Note = Synthetic Construct
aaaccacgacgctgaat

SEQ ID NO: 64 - DNA, 20 bp, Artificial Sequence
  Description of Artificial Sequence Note = Synthetic Construct
atttgtatcctcttcggctg

SEQ ID NO: 65 - DNA, 18 bp, Artificial Sequence
  Description of Artificial Sequence Note = Synthetic Construct
accaccatagtcatagcc

SEQ ID NO: 66 - DNA, 20 bp, Artificial Sequence
  Description of Artificial Sequence Note = Synthetic Construct
catacttggacaactgcttc

SEQ ID NO: 67 - DNA, 19 bp, Artificial Sequence
  Description of Artificial Sequence Note = Synthetic Construct
agcgttttacacctatccc

SEQ ID NO: 68 - DNA, 18 bp, Artificial Sequence
  Description of Artificial Sequence Note = Synthetic Construct
ccacgaagaaccagtagc

SEQ ID NO: 69 - DNA, 17 bp, Artificial Sequence
  Description of Artificial Sequence Note = Synthetic Construct
gtgtgggaaatcctgcg

SEQ ID NO: 70 - DNA, 17 bp, Artificial Sequence
  Description of Artificial Sequence Note = Synthetic Construct
gtggtggagccaagtct

SEQ ID NO: 71 - DNA, 18 bp, Artificial Sequence
  Description of Artificial Sequence Note = Synthetic Construct
ccgtacctgatgcacgaa

SEQ ID NO: 72 - DNA, 18 bp, Artificial Sequence
  Description of Artificial Sequence Note = Synthetic Construct
gtgcccgtagttgcgata

SEQ ID NO: 73 - DNA, 19 bp, Artificial Sequence
  Description of Artificial Sequence Note = Synthetic Construct
aagacactcaaccagaagg

SEQ ID NO: 74 - DNA, 23 bp, Artificial Sequence
  Description of Artificial Sequence Note = Synthetic Construct
ggtagagaacaaatgtgacaagg

SEQ ID NO: 75 - DNA, 18 bp, Artificial Sequence
  Description of Artificial Sequence Note = Synthetic Construct
aacaactacacgaacagc

SEQ ID NO: 76 - DNA, 18 bp, Artificial Sequence
  Description of Artificial Sequence Note = Synthetic Construct
attcttctgggtggtctc

SEQ ID NO: 77 - DNA, 18 bp, Artificial Sequence
  Description of Artificial Sequence Note = Synthetic Construct
ctgttgggcattctggac

SEQ ID NO: 78 - DNA, 18 bp, Artificial Sequence
  Description of Artificial Sequence Note = Synthetic Construct
ggaggctggtaaggaact

SEQ ID NO: 79 - DNA, 21 bp, Artificial Sequence
  Description of Artificial Sequence Note = Synthetic Construct
cgaccccatagaggaacataa

SEQ ID NO: 80 - DNA, 21 bp, Artificial Sequence
  Description of Artificial Sequence Note = Synthetic Construct
ttcttgacagaaaggaaagcg

SEQ ID NO: 81 - DNA, 18 bp, Artificial Sequence
  Description of Artificial Sequence Note = Synthetic Construct
cacttgggactgttgatg

SEQ ID NO: 82 - DNA, 19 bp, Artificial Sequence
  Description of Artificial Sequence Note = Synthetic Construct
tggataggaactcactggt

SEQ ID NO: 83 - DNA, 17 bp, Artificial Sequence
  Description of Artificial Sequence Note = Synthetic Construct
ccactgagtctcggcaa

SEQ ID NO: 84 - DNA, 17 bp, Artificial Sequence
  Description of Artificial Sequence Note = Synthetic Construct
atttcgtggtgggttct

SEQ ID NO: 85 - DNA, 17 bp, Artificial Sequence
  Description of Artificial Sequence Note = Synthetic Construct
tggaggagacttggtgt

SEQ ID NO: 86 - DNA, 20 bp, Artificial Sequence
  Description of Artificial Sequence Note = Synthetic Construct
gaatatgtggttctggctca

SEQ ID NO: 87 - DNA, 20 bp, Artificial Sequence
  Description of Artificial Sequence Note = Synthetic Construct
gtggaaatgcaggaactgaa

SEQ ID NO: 88 - DNA, 18 bp, Artificial Sequence
  Description of Artificial Sequence Note = Synthetic Construct
gctcgtcactcaagccaa

SEQ ID NO: 89 - DNA, 20 bp, Artificial Sequence
  Description of Artificial Sequence Note = Synthetic Construct
ggtgttgacatggacgataa

SEQ ID NO: 90 - DNA, 20 bp, Artificial Sequence
  Description of Artificial Sequence Note = Synthetic Construct
cttcccgctttcttttccta

SEQ ID NO: 91 - DNA, 22 bp, Artificial Sequence
  Description of Artificial Sequence Note = Synthetic Construct
gtttagaagcaatcagaggact

SEQ ID NO: 92 - DNA, 18 bp, Artificial Sequence
  Description of Artificial Sequence Note = Synthetic Construct
cctccacaaaggacaacc

SEQ ID NO: 93 - DNA, 17 bp, Artificial Sequence
  Description of Artificial Sequence Note = Synthetic Construct
tcagactccatgtgcct

SEQ ID NO: 94 - DNA, 21 bp, Artificial Sequence
  Description of Artificial Sequence Note = Synthetic Construct
cttcactgtccctatgacttc

SEQ ID NO: 95 - DNA, 19 bp, Artificial Sequence
  Description of Artificial Sequence Note = Synthetic Construct
cacactgcccaagtctcta

SEQ ID NO: 96 - DNA, 23 bp, Artificial Sequence
  Description of Artificial Sequence Note = Synthetic Construct
aagctgttgtcttctttgatacc

SEQ ID NO: 97 - DNA, 17 bp, Artificial Sequence
  Description of Artificial Sequence Note = Synthetic Construct
agcttggagactttggg

SEQ ID NO: 98 - DNA, 22 bp, Artificial Sequence
  Description of Artificial Sequence Note = Synthetic Construct
gtaataaggtgtgccaacaaat

SEQ ID NO: 99 - DNA, 20 bp, Artificial Sequence
  Description of Artificial Sequence Note = Synthetic Construct
gtcacagacaagtaatgtcg

SEQ ID NO: 100 - DNA, 17 bp, Artificial Sequence
  Description of Artificial Sequence Note = Synthetic Construct
tactgagtgtcaccgtt

SEQ ID NO: 101 - DNA, 23 bp, Artificial Sequence
  Description of Artificial Sequence Note = Synthetic Construct
cttactgtcattcgaagagagtt

SEQ ID NO: 102 - DNA, 19 bp, Artificial Sequence
  Description of Artificial Sequence Note = Synthetic Construct
atgcatccgaccttcaatc

SEQ ID NO: 103 - DNA, 20 bp, Artificial Sequence
  Description of Artificial Sequence Note = Synthetic Construct
aagcacatcaggtgaaaaat

SEQ ID NO: 104 - DNA, 17 bp, Artificial Sequence
  Description of Artificial Sequence Note = Synthetic Construct
taccacagccaatggca

SEQ ID NO: 105 - DNA, 20 bp, Artificial Sequence
  Description of Artificial Sequence Note = Synthetic Construct
acggaatcaagtcttctagc

SEQ ID NO: 106 - DNA, 19 bp, Artificial Sequence
  Description of Artificial Sequence Note = Synthetic Construct
tgccactgtttctggttac

SEQ ID NO: 107 - DNA, 22 bp, Artificial Sequence
  Description of Artificial Sequence Note = Synthetic Construct
gggatttgcattcagagatcag

SEQ ID NO: 108 - DNA, 18 bp, Artificial Sequence
  Description of Artificial Sequence Note = Synthetic Construct
ggaagggcatctcgtaag

SEQ ID NO: 109 - DNA, 18 bp, Artificial Sequence
  Description of Artificial Sequence Note = Synthetic Construct
ggcatggacatccagaag

SEQ ID NO: 110 - DNA, 17 bp, Artificial Sequence
  Description of Artificial Sequence Note = Synthetic Construct
ccacgacccggatgaat

SEQ ID NO: 111 - DNA, 19 bp, Artificial Sequence
  Description of Artificial Sequence Note = Synthetic Construct
tgaggtgtgcaccatgaac

SEQ ID NO: 112 - DNA, 20 bp, Artificial Sequence
  Description of Artificial Sequence Note = Synthetic Construct
cagaatgtgcttgccatagg

SEQ ID NO: 113 - DNA, 17 bp, Artificial Sequence
  Description of Artificial Sequence Note = Synthetic Construct
ttcctgggcatggagtc

SEQ ID NO: 114 - DNA, 18 bp, Artificial Sequence
  Description of Artificial Sequence Note = Synthetic Construct
caggtctttgcggatgtc

SEQ ID NO: 115 - DNA, 20 bp, Artificial Sequence
  Description of Artificial Sequence Note = Synthetic Construct
tggaaggactcatgaccaca

SEQ ID NO: 116 - DNA, 18 bp, Artificial Sequence
  Description of Artificial Sequence Note = Synthetic Construct
ggccatccacagtcttct

SEQ ID NO: 117 - DNA, 18 bp, Artificial Sequence
  Description of Artificial Sequence Note = Synthetic Construct
actatgcagcagacaagg

SEQ ID NO: 118 - DNA, 20 bp, Artificial Sequence
  Description of Artificial Sequence Note = Synthetic Construct
ccgtagtcgtgataccaaga

SEQ ID NO: 119 - DNA, 19 bp, Artificial Sequence
  Description of Artificial Sequence Note = Synthetic Construct
tctacaaccctgaagtgct

SEQ ID NO: 120 - DNA, 17 bp, Artificial Sequence
  Description of Artificial Sequence Note = Synthetic Construct
gacagacactggcaaca

SEQ ID NO: 121 - DNA, 20 bp, Artificial Sequence
  Description of Artificial Sequence Note = Synthetic Construct
gactattgctgtgatcgtct

SEQ ID NO: 122 - DNA, 21 bp, Artificial Sequence
  Description of Artificial Sequence Note = Synthetic Construct
ttacaatagcccaagtagcca

SEQ ID NO: 123 - DNA, 21 bp, Artificial Sequence
  Description of Artificial Sequence Note = Synthetic Construct
ggaagaggacttggagctact

SEQ ID NO: 124 - DNA, 19 bp, Artificial Sequence
  Description of Artificial Sequence Note = Synthetic Construct
tcctggacccgaggattat

SEQ ID NO: 125 - DNA, 17 bp, Artificial Sequence
  Description of Artificial Sequence Note = Synthetic Construct
acctggctgtgggaaga

SEQ ID NO: 126 - DNA, 17 bp, Artificial Sequence
  Description of Artificial Sequence Note = Synthetic Construct
gccctcacccagatact

SEQ ID NO: 127 - DNA, 22 bp, Artificial Sequence
  Description of Artificial Sequence Note = Synthetic Construct
cgggagattgctggacatataa

SEQ ID NO: 128 - DNA, 17 bp, Artificial Sequence
  Description of Artificial Sequence Note = Synthetic Construct
tggcacgctccagtttc

SEQ ID NO: 129 - DNA, 18 bp, Artificial Sequence
  Description of Artificial Sequence Note = Synthetic Construct
cgatgatgaggtgtacgc

SEQ ID NO: 130 - DNA, 17 bp, Artificial Sequence
  Description of Artificial Sequence Note = Synthetic Construct
gctcagccaactgcttc

SEQ ID NO: 131 - DNA, 20 bp, Artificial Sequence
  Description of Artificial Sequence Note = Synthetic Construct
tggcactcatctacctcatc

SEQ ID NO: 132 - DNA, 18 bp, Artificial Sequence
  Description of Artificial Sequence Note = Synthetic Construct
ccacgaagaaccagtagc

SEQ ID NO: 133 - DNA, 17 bp, Artificial Sequence
  Description of Artificial Sequence Note = Synthetic Construct
gcgcacagagctctcag

SEQ ID NO: 134 - DNA, 18 bp, Artificial Sequence
  Description of Artificial Sequence Note = Synthetic Construct
agggctggagatttttgc

SEQ ID NO: 135 - DNA, 21 bp, Artificial Sequence
  Description of Artificial Sequence Note = Synthetic Construct
tccaccagtaaaaccagactt

SEQ ID NO: 136 - DNA, 21 bp, Artificial Sequence
  Description of Artificial Sequence Note = Synthetic Construct
ttgtgaccctgggattagatg

SEQ ID NO: 137 - DNA, 17 bp, Artificial Sequence
  Description of Artificial Sequence Note = Synthetic Construct
gctggctgagcagaaag

SEQ ID NO: 138 - DNA, 22 bp, Artificial Sequence
  Description of Artificial Sequence Note = Synthetic Construct
ttcctccatcaagagttcaaca

SEQ ID NO: 139 - DNA, 18 bp, Artificial Sequence
  Description of Artificial Sequence Note = Synthetic Construct
agccgcaagtcctgtgta

SEQ ID NO: 140 - DNA, 18 bp, Artificial Sequence
  Description of Artificial Sequence Note = Synthetic Construct
gggtgccacattggaaga

SEQ ID NO: 141 - DNA, 17 bp, Artificial Sequence
  Description of Artificial Sequence Note = Synthetic Construct
ccccaagggatgcgtat

SEQ ID NO: 142 - DNA, 17 bp, Artificial Sequence
  Description of Artificial Sequence Note = Synthetic Construct
agatgtcgctgagggtt

SEQ ID NO: 143 - DNA, 19 bp, Artificial Sequence
  Description of Artificial Sequence Note = Synthetic Construct
gctcaggttcatgatggat

SEQ ID NO: 144 - DNA, 17 bp, Artificial Sequence
  Description of Artificial Sequence Note = Synthetic Construct
gatggcgaccaagttct

SEQ ID NO: 145 - DNA, 18 bp, Artificial Sequence
  Description of Artificial Sequence Note = Synthetic Construct
gagcaagcgggttgttat

SEQ ID NO: 146 - DNA, 20 bp, Artificial Sequence
  Description of Artificial Sequence Note = Synthetic Construct
tctttgggggagttagacac

SEQ ID NO: 147 - DNA, 24 bp, Artificial Sequence
  Description of Artificial Sequence Note = Synthetic Construct
tcccaaactgaaaattctcttgtc

SEQ ID NO: 148 - DNA, 24 bp, Artificial Sequence
  Description of Artificial Sequence Note = Synthetic Construct
gtgatgtagaagaatccaccatag

SEQ ID NO: 149 - DNA, 17 bp, Artificial Sequence
  Description of Artificial Sequence Note = Synthetic Construct
tcgctcacgggtcttac

SEQ ID NO: 150 - DNA, 22 bp, Artificial Sequence
  Description of Artificial Sequence Note = Synthetic Construct
ttcatctctccaatctcaccag

SEQ ID NO: 151 - DNA, 22 bp, Artificial Sequence
  Description of Artificial Sequence Note = Synthetic Construct
gcaaaatggttatgacccttac

SEQ ID NO: 152 - DNA, 23 bp, Artificial Sequence
  Description of Artificial Sequence Note = Synthetic Construct
caggaacatttttatgccttctc

SEQ ID NO: 153 - DNA, 18 bp, Artificial Sequence
  Description of Artificial Sequence Note = Synthetic Construct
tccgaggccagtaagttg

SEQ ID NO: 154 - DNA, 17 bp, Artificial Sequence
  Description of Artificial Sequence Note = Synthetic Construct
tgtctctgctgctgtcc

SEQ ID NO: 155 - DNA, 20 bp, Artificial Sequence
  Description of Artificial Sequence Note = Synthetic Construct
cccaacaagaaagtcagcaa

SEQ ID NO: 156 - DNA, 18 bp, Artificial Sequence
  Description of Artificial Sequence Note = Synthetic Construct
aggtccaggatgtagtcg

SEQ ID NO: 157 - DNA, 19 bp, Artificial Sequence
  Description of Artificial Sequence Note = Synthetic Construct
tgccgaaatgttatcgcag

SEQ ID NO: 158 - DNA, 17 bp, Artificial Sequence
  Description of Artificial Sequence Note = Synthetic Construct
tggtgagggctccttga

SEQ ID NO: 159 - DNA, 20 bp, Artificial Sequence
  Description of Artificial Sequence Note = Synthetic Construct
ggcagcactttcttcctaca

SEQ ID NO: 160 - DNA, 18 bp, Artificial Sequence
  Description of Artificial Sequence Note = Synthetic Construct
gcggttcatttggagtct

SEQ ID NO: 161 - DNA, 18 bp, Artificial Sequence
  Description of Artificial Sequence Note = Synthetic Construct
tcttcagcggctctctca

SEQ ID NO: 162 - DNA, 17 bp, Artificial Sequence
  Description of Artificial Sequence Note = Synthetic Construct
tcagggaaccaaggtcg

SEQ ID NO: 163 - DNA, 19 bp, Artificial Sequence
  Description of Artificial Sequence Note = Synthetic Construct
tggcagacaaaccagacat

SEQ ID NO: 164 - DNA, 17 bp, Artificial Sequence
  Description of Artificial Sequence Note = Synthetic Construct
tccgcttctcctgctca

SEQ ID NO: 165 - DNA, 21 bp, Artificial Sequence
  Description of Artificial Sequence Note = Synthetic Construct
cttgctcacttccgttatttg

SEQ ID NO: 166 - DNA, 21 bp, Artificial Sequence
  Description of Artificial Sequence Note = Synthetic Construct
cgggacacattgatctttaca

SEQ ID NO: 167 - DNA, 22 bp, Artificial Sequence
  Description of Artificial Sequence Note = Synthetic Construct
ctggaagagttgaataaagagc

SEQ ID NO: 168 - DNA, 17 bp, Artificial Sequence
  Description of Artificial Sequence Note = Synthetic Construct
gcaaatccttgggcaga

SEQ ID NO: 169 - DNA, 19 bp, Artificial Sequence
  Description of Artificial Sequence Note = Synthetic Construct
ggacgctcctgattatgac

SEQ ID NO: 170 - DNA, 17 bp, Artificial Sequence
  Description of Artificial Sequence Note = Synthetic Construct
agggcagattgggaaag

SEQ ID NO: 171 - DNA, 18 bp, Artificial Sequence
  Description of Artificial Sequence Note = Synthetic Construct
aggggtgccctctgagat

SEQ ID NO: 172 - DNA, 21 bp, Artificial Sequence
  Description of Artificial Sequence Note = Synthetic Construct
tcacagggtcaaacttccagt

SEQ ID NO: 173 - DNA, 17 bp, Artificial Sequence
  Description of Artificial Sequence Note = Synthetic Construct
gggcagagcttcagttg

SEQ ID NO: 174 - DNA, 17 bp, Artificial Sequence
  Description of Artificial Sequence Note = Synthetic Construct
ctggagcctcagggaga

SEQ ID NO: 175 - DNA, 18 bp, Artificial Sequence
  Description of Artificial Sequence Note = Synthetic Construct
tctccagccacctcatac

SEQ ID NO: 176 - DNA, 19 bp, Artificial Sequence
  Description of Artificial Sequence Note = Synthetic Construct
tattaccgaggcgaagagt

SEQ ID NO: 177 - DNA, 17 bp, Artificial Sequence
  Description of Artificial Sequence Note = Synthetic Construct
gtggtgcctgtcagcaa

SEQ ID NO: 178 - DNA, 18 bp, Artificial Sequence
  Description of Artificial Sequence Note = Synthetic Construct
cctctcagcctctctgtg

SEQ ID NO: 179 - DNA, 20 bp, Artificial Sequence
  Description of Artificial Sequence Note = Synthetic Construct
gctcccagcttcttcagaga

SEQ ID NO: 180 - DNA, 23 bp, Artificial Sequence
  Description of Artificial Sequence Note = Synthetic Construct
gaggatccttgggttataattgg

SEQ ID NO: 181 - DNA, 18 bp, Artificial Sequence
  Description of Artificial Sequence Note = Synthetic Construct
gagtgctggtgtgtgaac

SEQ ID NO: 182 - DNA, 21 bp, Artificial Sequence
  Description of Artificial Sequence Note = Synthetic Construct
tgtagaagagatgacactcgg

SEQ ID NO: 183 - DNA, 18 bp, Artificial Sequence
  Description of Artificial Sequence Note = Synthetic Construct
gactccaagcgcgaaaac

SEQ ID NO: 184 - DNA, 23 bp, Artificial Sequence
  Description of Artificial Sequence Note = Synthetic Construct
cagacatgttggtattgcacatt

SEQ ID NO: 185 - DNA, 22 bp, Artificial Sequence
  Description of Artificial Sequence Note = Synthetic Construct
gggaagtaaggaccagagacaa

SEQ ID NO: 186 - DNA, 25 bp, Artificial Sequence
  Description of Artificial Sequence Note = Synthetic Construct
tccagatgattctttaacaggtagc

SEQ ID NO: 187 - DNA, 20 bp, Artificial Sequence
  Description of Artificial Sequence Note = Synthetic Construct
aggccttggaactcaaggat

SEQ ID NO: 188 - DNA, 20 bp, Artificial Sequence
  Description of Artificial Sequence Note = Synthetic Construct
ccctttttggacttcaggtg

SEQ ID NO: 189 - DNA, 20 bp, Artificial Sequence
  Description of Artificial Sequence Note = Synthetic Construct
gtaaatcaccttctgagcct

SEQ ID NO: 190 - DNA, 23 bp, Artificial Sequence
  Description of Artificial Sequence Note = Synthetic Construct
acttgggatatgtgaataagacc

SEQ ID NO: 191 - DNA, 18 bp, Artificial Sequence
  Description of Artificial Sequence Note = Synthetic Construct
gccctctctgtttctccc

SEQ ID NO: 192 - DNA, 20 bp, Artificial Sequence
  Description of Artificial Sequence Note = Synthetic Construct
ggattcaagtaggatgctgc

SEQ ID NO: 193 - DNA, 20 bp, Artificial Sequence
  Description of Artificial Sequence Note = Synthetic Construct
gatcgtagcccctatgagac

SEQ ID NO: 194 - DNA, 19 bp, Artificial Sequence
  Description of Artificial Sequence Note = Synthetic Construct
actgctgggtttgtgtaag

SEQ ID NO: 195 - DNA, 20 bp, Artificial Sequence
  Description of Artificial Sequence Note = Synthetic Construct
cagcttctgcacacatgacc

SEQ ID NO: 196 - DNA, 19 bp, Artificial Sequence
  Description of Artificial Sequence Note = Synthetic Construct
cgtcgatgcctatgacagc

SEQ ID NO: 197 - DNA, 19 bp, Artificial Sequence
  Description of Artificial Sequence Note = Synthetic Construct
gggagacgacaaagtgaag

SEQ ID NO: 198 - DNA, 19 bp, Artificial Sequence
  Description of Artificial Sequence Note = Synthetic Construct
ataccaggagcaagctacc

SEQ ID NO: 199 - DNA, 19 bp, Artificial Sequence
  Description of Artificial Sequence Note = Synthetic Construct
gatcgtcctgcagaatctc

SEQ ID NO: 200 - DNA, 18 bp, Artificial Sequence
  Description of Artificial Sequence Note = Synthetic Construct
gggtcctcatcatcgttg

SEQ ID NO: 201 - DNA, 17 bp, Artificial Sequence
  Description of Artificial Sequence Note = Synthetic Construct
actcggttggagagact

SEQ ID NO: 202 - DNA, 17 bp, Artificial Sequence
  Description of Artificial Sequence Note = Synthetic Construct
ggctttccctttcccat

SEQ ID NO: 203 - DNA, 22 bp, Artificial Sequence
  Description of Artificial Sequence Note = Synthetic Construct
tggatttaccttatcccctcaa

SEQ ID NO: 204 - DNA, 20 bp, Artificial Sequence
  Description of Artificial Sequence Note = Synthetic Construct
tggctttggtcgttctgttt

SEQ ID NO: 205 - DNA, 22 bp, Artificial Sequence
  Description of Artificial Sequence Note = Synthetic Construct
ggacctgaaatccaaggaagat

SEQ ID NO: 206 - DNA, 21 bp, Artificial Sequence
  Description of Artificial Sequence Note = Synthetic Construct
cagtcacagatggttttgcac

SEQ ID NO: 207 - DNA, 18 bp, Artificial Sequence
  Description of Artificial Sequence Note = Synthetic Construct
aacgtccagagtgctaca

SEQ ID NO: 208 - DNA, 20 bp, Artificial Sequence
  Description of Artificial Sequence Note = Synthetic Construct
ccaagcatgacttcagattc

SEQ ID NO: 209 - DNA, 19 bp, Artificial Sequence
  Description of Artificial Sequence Note = Synthetic Construct
tcagccgctacttccaata

SEQ ID NO: 210 - DNA, 21 bp, Artificial Sequence
  Description of Artificial Sequence Note = Synthetic Construct
ctcagcatcactgtctggtta

SEQ ID NO: 211 - DNA, 21 bp, Artificial Sequence
  Description of Artificial Sequence Note = Synthetic Construct
ccctagagggcaagtacgagt

SEQ ID NO: 212 - DNA, 20 bp, Artificial Sequence
  Description of Artificial Sequence Note = Synthetic Construct
agtagaactcgggcaagctg

SEQ ID NO: 213 - DNA, 19 bp, Artificial Sequence
  Description of Artificial Sequence Note = Synthetic Construct
ctcaccgtccctgtacctg

SEQ ID NO: 214 - DNA, 19 bp, Artificial Sequence
  Description of Artificial Sequence Note = Synthetic Construct
caggagtgggcgttttctt

SEQ ID NO: 215 - DNA, 19 bp, Artificial Sequence
  Description of Artificial Sequence Note = Synthetic Construct
actctcatctccaggaacc

SEQ ID NO: 216 - DNA, 18 bp, Artificial Sequence
  Description of Artificial Sequence Note = Synthetic Construct
ctcgcaacgatcttcgtc

SEQ ID NO: 217 - DNA, 18 bp, Artificial Sequence
  Description of Artificial Sequence Note = Synthetic Construct
gcagtcttccaacccaat

SEQ ID NO: 218 - DNA, 18 bp, Artificial Sequence
  Description of Artificial Sequence Note = Synthetic Construct
gaggacatggtgtgcatc

SEQ ID NO: 219 - DNA, 18 bp, Artificial Sequence
  Description of Artificial Sequence Note = Synthetic Construct
gcttgggccatcacaaat

SEQ ID NO: 220 - DNA, 20 bp, Artificial Sequence
  Description of Artificial Sequence Note = Synthetic Construct
cggcttgatacaacccagtt

SEQ ID NO: 221 - DNA, 22 bp, Artificial Sequence
  Description of Artificial Sequence Note = Synthetic Construct
tggacgaatatgatccaacaat

SEQ ID NO: 222 - DNA, 21 bp, Artificial Sequence
  Description of Artificial Sequence Note = Synthetic Construct
tccctcattgcactgtactcc

SEQ ID NO: 223 - DNA, 19 bp, Artificial Sequence
  Description of Artificial Sequence Note = Synthetic Construct
ccagaatcactgaagggtc

SEQ ID NO: 224 - DNA, 17 bp, Artificial Sequence
  Description of Artificial Sequence Note = Synthetic Construct
aggaagtcatcgcacac

SEQ ID NO: 225 - DNA, 19 bp, Artificial Sequence
  Description of Artificial Sequence Note = Synthetic Construct
aaccctttgagaacacgac

SEQ ID NO: 226 - DNA, 19 bp, Artificial Sequence
  Description of Artificial Sequence Note = Synthetic Construct
tctttccgttcatctgcca

SEQ ID NO: 227 - DNA, 18 bp, Artificial Sequence
  Description of Artificial Sequence Note = Synthetic Construct
tgtggctcattaggcaac

SEQ ID NO: 228 - DNA, 17 bp, Artificial Sequence
  Description of Artificial Sequence Note = Synthetic Construct
cttcgactggactctgt

SEQ ID NO: 229 - DNA, 19 bp, Artificial Sequence
  Description of Artificial Sequence Note = Synthetic Construct
tttctggcagtagcaagag

SEQ ID NO: 230 - DNA, 18 bp, Artificial Sequence
  Description of Artificial Sequence Note = Synthetic Construct
acttgtgtctggttcagg

SEQ ID NO: 231 - DNA, 22 bp, Artificial Sequence
  Description of Artificial Sequence Note = Synthetic Construct
gctgaagccctatgaatcattt

SEQ ID NO: 232 - DNA, 21 bp, Artificial Sequence
  Description of Artificial Sequence Note = Synthetic Construct
tccaactctgcagacatttcc

SEQ ID NO: 233 - DNA, 20 bp, Artificial Sequence
  Description of Artificial Sequence Note = Synthetic Construct
cagtggagagaagaagttgc

SEQ ID NO: 234 - DNA, 18 bp, Artificial Sequence
  Description of Artificial Sequence Note = Synthetic Construct
ctggtgctcatagtcacg

SEQ ID NO: 235 - DNA, 18 bp, Artificial Sequence
  Description of Artificial Sequence Note = Synthetic Construct
caaagcgcaccaggaaac

SEQ ID NO: 236 - DNA, 22 bp, Artificial Sequence
  Description of Artificial Sequence Note = Synthetic Construct
gttgttctgagctgttgttcgt

SEQ ID NO: 237 - DNA, 18 bp, Artificial Sequence
  Description of Artificial Sequence Note = Synthetic Construct
gcagcctgggaacttcag

SEQ ID NO: 238 - DNA, 20 bp, Artificial Sequence
  Description of Artificial Sequence Note = Synthetic Construct
caccagcttctttcggattg

SEQ ID NO: 239 - DNA, 19 bp, Artificial Sequence
  Description of Artificial Sequence Note = Synthetic Construct
gagtcgctttgtgtaaccg

SEQ ID NO: 240 - DNA, 23 bp, Artificial Sequence
  Description of Artificial Sequence Note = Synthetic Construct
tgagaatgcaggatctttaacag

SEQ ID NO: 241 - DNA, 20 bp, Artificial Sequence
  Description of Artificial Sequence Note = Synthetic Construct
tgctgacgatgatgaaggag

SEQ ID NO: 242 - DNA, 20 bp, Artificial Sequence
  Description of Artificial Sequence Note = Synthetic Construct
cgaggtaatttgtgcccttt

SEQ ID NO: 243 - DNA, 19 bp, Artificial Sequence
  Description of Artificial Sequence Note = Synthetic Construct
ctggagaaagccttgaact

SEQ ID NO: 244 - DNA, 19 bp, Artificial Sequence
  Description of Artificial Sequence Note = Synthetic Construct
ctgtagacggcatggaaat

SEQ ID NO: 245 - DNA, 20 bp, Artificial Sequence
  Description of Artificial Sequence Note = Synthetic Construct
gtgcgaaaagatctgcaaaa

SEQ ID NO: 246 - DNA, 20 bp, Artificial Sequence
  Description of Artificial Sequence Note = Synthetic Construct
tcagctgcttgtctgcattt

SEQ ID NO: 247 - DNA, 20 bp, Artificial Sequence
  Description of Artificial Sequence Note = Synthetic Construct
ggggtttcctacttggttcg

SEQ ID NO: 248 - DNA, 18 bp, Artificial Sequence
  Description of Artificial Sequence Note = Synthetic Construct
ccgggtgttgaagtccag

SEQ ID NO: 249 - DNA, 17 bp, Artificial Sequence
  Description of Artificial Sequence Note = Synthetic Construct
gccaggagacggagttt

SEQ ID NO: 250 - DNA, 19 bp, Artificial Sequence
  Description of Artificial Sequence Note = Synthetic Construct
cgtgtcctctgttacccga

SEQ ID NO: 251 - DNA, 19 bp, Artificial Sequence
  Description of Artificial Sequence Note = Synthetic Construct
cactctccatgccatccac

SEQ ID NO: 252 - DNA, 19 bp, Artificial Sequence
  Description of Artificial Sequence Note = Synthetic Construct
gcccaacctcgctaagaaa

SEQ ID NO: 253 - DNA, 17 bp, Artificial Sequence
  Description of Artificial Sequence Note = Synthetic Construct
tggaggagacttggtgt

SEQ ID NO: 254 - DNA, 20 bp, Artificial Sequence
  Description of Artificial Sequence Note = Synthetic Construct
gaatatgtggttctggctca

SEQ ID NO: 255 - DNA, 20 bp, Artificial Sequence
  Description of Artificial Sequence Note = Synthetic Construct
gtggaaatgcaggaactgaa

SEQ ID NO: 256 - DNA, 18 bp, Artificial Sequence
  Description of Artificial Sequence Note = Synthetic Construct
gctcgtcactcaagccaa

SEQ ID NO: 257 - DNA, 21 bp, Artificial Sequence
  Description of Artificial Sequence Note = Synthetic Construct
ggatcattacaagttggctct

SEQ ID NO: 258 - DNA, 22 bp, Artificial Sequence
  Description of Artificial Sequence Note = Synthetic Construct
cttcatcagtcgcacataatct

SEQ ID NO: 259 - DNA, 20 bp, Artificial Sequence
  Description of Artificial Sequence Note = Synthetic Construct
tggaattggacaagactccc

SEQ ID NO: 260 - DNA, 19 bp, Artificial Sequence
  Description of Artificial Sequence Note = Synthetic Construct
acgctgagagataaggatg

SEQ ID NO: 261 - DNA, 17 bp, Artificial Sequence
  Description of Artificial Sequence Note = Synthetic Construct
ccactctcttcaacggt

SEQ ID NO: 262 - DNA, 17 bp, Artificial Sequence
  Description of Artificial Sequence Note = Synthetic Construct
agtgtcccatatccgca

SEQ ID NO: 263 - DNA, 17 bp, Artificial Sequence
  Description of Artificial Sequence Note = Synthetic Construct
gctactacgcagacacg

SEQ ID NO: 264 - DNA, 20 bp, Artificial Sequence
  Description of Artificial Sequence Note = Synthetic Construct
ctgagttcatgttgctgacc

SEQ ID NO: 265 - DNA, 19 bp, Artificial Sequence
  Description of Artificial Sequence Note = Synthetic Construct
cattaagcccaagcgaagg

SEQ ID NO: 266 - DNA, 18 bp, Artificial Sequence
  Description of Artificial Sequence Note = Synthetic Construct
tgacagttcgcacaggac

SEQ ID NO: 267 - DNA, 18 bp, Artificial Sequence
  Description of Artificial Sequence Note = Synthetic Construct
ggaggcggaagaaaccag

SEQ ID NO: 268 - DNA, 20 bp, Artificial Sequence
  Description of Artificial Sequence Note = Synthetic Construct
ggggaaagacaaagtttcca

SEQ ID NO: 269 - DNA, 22 bp, Artificial Sequence
  Description of Artificial Sequence Note = Synthetic Construct
tttgtcctcctcaatctggttt

SEQ ID NO: 270 - DNA, 22 bp, Artificial Sequence
  Description of Artificial Sequence Note = Synthetic Construct
cataatcctggggacatactgg

SEQ ID NO: 271 - DNA, 18 bp, Artificial Sequence
  Description of Artificial Sequence Note = Synthetic Construct
gcagggagaggagtttgt

SEQ ID NO: 272 - DNA, 18 bp, Artificial Sequence
  Description of Artificial Sequence Note = Synthetic Construct
gacttcagggtgctggac

SEQ ID NO: 273 - DNA, 17 bp, Artificial Sequence
  Description of Artificial Sequence Note = Synthetic Construct
gttccaggtcccatacg

SEQ ID NO: 274 - DNA, 17 bp, Artificial Sequence
  Description of Artificial Sequence Note = Synthetic Construct
tagagcctgccatctcg

SEQ ID NO: 275 - DNA, 19 bp, Artificial Sequence
  Description of Artificial Sequence Note = Synthetic Construct
gtttgcggttcaggtttgg

SEQ ID NO: 276 - DNA, 19 bp, Artificial Sequence
  Description of Artificial Sequence Note = Synthetic Construct
catgtgggcttcaagcatc

SEQ ID NO: 277 - DNA, 23 bp, Artificial Sequence
  Description of Artificial Sequence Note = Synthetic Construct
tcgaactgaaggctatttacgag

SEQ ID NO: 278 - DNA, 21 bp, Artificial Sequence
  Description of Artificial Sequence Note = Synthetic Construct
ctgctgagaatcaaagtggga

SEQ ID NO: 279 - DNA, 19 bp, Artificial Sequence
  Description of Artificial Sequence Note = Synthetic Construct
aactccgaagcctccctta

SEQ ID NO: 280 - DNA, 18 bp, Artificial Sequence
  Description of Artificial Sequence Note = Synthetic Construct
gtgtttgtgcgctgaatc

SEQ ID NO: 281 - DNA, 19 bp, Artificial Sequence
  Description of Artificial Sequence Note = Synthetic Construct
ccagggtttgtgtatttgc

SEQ ID NO: 282 - DNA, 19 bp, Artificial Sequence
  Description of Artificial Sequence Note = Synthetic Construct
actgaagaaccgaagatgg

SEQ ID NO: 283 - DNA, 18 bp, Artificial Sequence
  Description of Artificial Sequence Note = Synthetic Construct
tgggtcgtgtcaggaaac

SEQ ID NO: 284 - DNA, 18 bp, Artificial Sequence
  Description of Artificial Sequence Note = Synthetic Construct
caccgctggaaactgaac

SEQ ID NO: 285 - DNA, 20 bp, Artificial Sequence
  Description of Artificial Sequence Note = Synthetic Construct
gcctgggttgtctctacagg

SEQ ID NO: 286 - DNA, 20 bp, Artificial Sequence
  Description of Artificial Sequence Note = Synthetic Construct
ccccagggttactgtgtgtc

SEQ ID NO: 287 - DNA, 20 bp, Artificial Sequence
  Description of Artificial Sequence Note = Synthetic Construct
gctggctctcacactgatag

SEQ ID NO: 288 - DNA, 20 bp, Artificial Sequence
  Description of Artificial Sequence Note = Synthetic Construct
gcccttacacatcggagaac

SEQ ID NO: 289 - DNA, 20 bp, Artificial Sequence
  Description of Artificial Sequence Note = Synthetic Construct
gcaggtccagaagttcgatg

SEQ ID NO: 290 - DNA, 21 bp, Artificial Sequence
  Description of Artificial Sequence Note = Synthetic Construct
ccgcagtacagacttgcactt

SEQ ID NO: 291 - DNA, 21 bp, Artificial Sequence
  Description of Artificial Sequence Note = Synthetic Construct
gctccaaggagaacttcatac

SEQ ID NO: 292 - DNA, 20 bp, Artificial Sequence
  Description of Artificial Sequence Note = Synthetic Construct
cttgcaatctcttaatgccc

SEQ ID NO: 293 - DNA, 20 bp, Artificial Sequence
  Description of Artificial Sequence Note = Synthetic Construct
gcacaaagccattctaagtc

SEQ ID NO: 294 - DNA, 22 bp, Artificial Sequence
  Description of Artificial Sequence Note = Synthetic Construct
gacgcttcctatcactctattc

SEQ ID NO: 295 - DNA, 21 bp, Artificial Sequence
  Description of Artificial Sequence Note = Synthetic Construct
acagccactttcagaagcaag

SEQ ID NO: 296 - DNA, 24 bp, Artificial Sequence
  Description of Artificial Sequence Note = Synthetic Construct
cgatggttttgtacaagatttctc

SEQ ID NO: 297 - DNA, 20 bp, Artificial Sequence
  Description of Artificial Sequence Note = Synthetic Construct
cctcacgaattgctgaactt

SEQ ID NO: 298 - DNA, 20 bp, Artificial Sequence
  Description of Artificial Sequence Note = Synthetic Construct
ccacagtctgtgataaacgg

SEQ ID NO: 299 - DNA, 20 bp, Artificial Sequence
  Description of Artificial Sequence Note = Synthetic Construct
cgagttcttcagcgatctac

SEQ ID NO: 300 - DNA, 18 bp, Artificial Sequence
  Description of Artificial Sequence Note = Synthetic Construct
agccatgcagaacaacag

SEQ ID NO: 301 - DNA, 18 bp, Artificial Sequence
  Description of Artificial Sequence Note = Synthetic Construct
cccttccatgcgtccata

SEQ ID NO: 302 - DNA, 20 bp, Artificial Sequence
  Description of Artificial Sequence Note = Synthetic Construct
tgtcagcacaaactgaagca

SEQ ID NO: 303 - DNA, 18 bp, Artificial Sequence
  Description of Artificial Sequence Note = Synthetic Construct
gtcgaagccgcaattagg

SEQ ID NO: 304 - DNA, 19 bp, Artificial Sequence
  Description of Artificial Sequence Note = Synthetic Construct
ggaacaaactgctctgcca

SEQ ID NO: 305 - DNA, 19 bp, Artificial Sequence
  Description of Artificial Sequence Note = Synthetic Construct
ggagtttgggttccatctt

SEQ ID NO: 306 - DNA, 22 bp, Artificial Sequence
  Description of Artificial Sequence Note = Synthetic Construct
ttctctgccacttaaatcctcg

SEQ ID NO: 307 - DNA, 19 bp, Artificial Sequence
  Description of Artificial Sequence Note = Synthetic Construct
tgtccatcttgtcgtcttc

SEQ ID NO: 308 - DNA, 20 bp, Artificial Sequence
  Description of Artificial Sequence Note = Synthetic Construct
ctccttcctcctgtagtttc

SEQ ID NO: 309 - DNA, 18 bp, Artificial Sequence
  Description of Artificial Sequence Note = Synthetic Construct
gtaccagtggaaggcaac

SEQ ID NO: 310 - DNA, 18 bp, Artificial Sequence
  Description of Artificial Sequence Note = Synthetic Construct
acactgctctcctccatc

SEQ ID NO: 311 - DNA, 20 bp, Artificial Sequence
  Description of Artificial Sequence Note = Synthetic Construct
ctgcacccagtgtttctgtc

SEQ ID NO: 312 - DNA, 20 bp, Artificial Sequence
  Description of Artificial Sequence Note = Synthetic Construct
gagagtccccggtatcatcc

SEQ ID NO: 313 - DNA, 18 bp, Artificial Sequence
  Description of Artificial Sequence Note = Synthetic Construct
gcactgtgccgcttatag

SEQ ID NO: 314 - DNA, 18 bp, Artificial Sequence
  Description of Artificial Sequence Note = Synthetic Construct
tcgggcatacccatcttc

SEQ ID NO: 315 - DNA, 18 bp, Artificial Sequence
  Description of Artificial Sequence Note = Synthetic Construct
gagtcgagcccgctatga

SEQ ID NO: 316 - DNA, 18 bp, Artificial Sequence
  Description of Artificial Sequence Note = Synthetic Construct
caacccagtgcattgacg

SEQ ID NO: 317 - DNA, 18 bp, Artificial Sequence
  Description of Artificial Sequence Note = Synthetic Construct
gtggaatgcctgctgacc

SEQ ID NO: 318 - DNA, 19 bp, Artificial Sequence
  Description of Artificial Sequence Note = Synthetic Construct
cgcactccagcacctagac

SEQ ID NO: 319 - DNA, 24 bp, Artificial Sequence
  Description of Artificial Sequence Note = Synthetic Construct
agttggtgcacaaaatactgtcat

SEQ ID NO: 320 - DNA, 20 bp, Artificial Sequence
  Description of Artificial Sequence Note = Synthetic Construct
tcccaagttttgagccattc

SEQ ID NO: 321 - DNA, 19 bp, Artificial Sequence
  Description of Artificial Sequence Note = Synthetic Construct
ctgtctgagtgccgtggat

SEQ ID NO: 322 - DNA, 20 bp, Artificial Sequence
  Description of Artificial Sequence Note = Synthetic Construct
tccttgtaatggggagacca

SEQ ID NO: 323 - DNA, 20 bp, Artificial Sequence
  Description of Artificial Sequence Note = Synthetic Construct
tgcctatgtgacgacaatcc

SEQ ID NO: 324 - DNA, 20 bp, Artificial Sequence
  Description of Artificial Sequence Note = Synthetic Construct
ggctgcaatttgcacagttc

SEQ ID NO: 325 - DNA, 21 bp, Artificial Sequence
  Description of Artificial Sequence Note = Synthetic Construct
aaagaggaagaccgtggatgg

SEQ ID NO: 326 - DNA, 20 bp, Artificial Sequence
  Description of Artificial Sequence Note = Synthetic Construct
cactgggcgaattatatgcg

SEQ ID NO: 327 - DNA, 18 bp, Artificial Sequence
  Description of Artificial Sequence Note = Synthetic Construct
ccagtagcattgtccgag

SEQ ID NO: 328 - DNA, 19 bp, Artificial Sequence
  Description of Artificial Sequence Note = Synthetic Construct
cccatttgtctgtcttcac

SEQ ID NO: 329 - DNA, 19 bp, Artificial Sequence
  Description of Artificial Sequence Note = Synthetic Construct
ctggaacagcaagtggtag

SEQ ID NO: 330 - DNA, 19 bp, Artificial Sequence
  Description of Artificial Sequence Note = Synthetic Construct
gccatgagttttctctcgt

SEQ ID NO: 331 - DNA, 25 bp, Artificial Sequence
  Description of Artificial Sequence Note = Synthetic Construct
tcaagtaaaatcaagctgggtaatc

SEQ ID NO: 332 - DNA, 20 bp, Artificial Sequence
  Description of Artificial Sequence Note = Synthetic Construct
taggactgggactgccgtaa

SEQ ID NO: 333 - DNA, 20 bp, Artificial Sequence
  Description of Artificial Sequence Note = Synthetic Construct
acaagggacactcaaactac

SEQ ID NO: 334 - DNA, 20 bp, Artificial Sequence
  Description of Artificial Sequence Note = Synthetic Construct
tgtttaggagttcttcgcag

SEQ ID NO: 335 - DNA, 18 bp, Artificial Sequence
  Description of Artificial Sequence Note = Synthetic Construct
cgtggcagatgtgaacga

SEQ ID NO: 336 - DNA, 17 bp, Artificial Sequence
  Description of Artificial Sequence Note = Synthetic Construct
agtgggcatcccgtaga

SEQ ID NO: 337 - DNA, 20 bp, Artificial Sequence
  Description of Artificial Sequence Note = Synthetic Construct
tgccctgtatgatgtcagga

SEQ ID NO: 338 - DNA, 23 bp, Artificial Sequence
  Description of Artificial Sequence Note = Synthetic Construct
gggactatcaatgttgggttctc

SEQ ID NO: 339 - DNA, 20 bp, Artificial Sequence
  Description of Artificial Sequence Note = Synthetic Construct
accgacagggatcacttcat

SEQ ID NO: 340 - DNA, 21 bp, Artificial Sequence
  Description of Artificial Sequence Note = Synthetic Construct
agccgacactcttcatcagtc

SEQ ID NO: 341 - DNA, 20 bp, Artificial Sequence
  Description of Artificial Sequence Note = Synthetic Construct
ttgaacccacccaaatacct

SEQ ID NO: 342 - DNA, 20 bp, Artificial Sequence
  Description of Artificial Sequence Note = Synthetic Construct
gatgcagaaggatggctttt

SEQ ID NO: 343 - DNA, 24 bp, Artificial Sequence
  Description of Artificial Sequence Note = Synthetic Construct
ccaacaaaatattcatggttcttg

SEQ ID NO: 344 - DNA, 20 bp, Artificial Sequence
  Description of Artificial Sequence Note = Synthetic Construct
aggcgatcctgggaaattat

SEQ ID NO: 345 - DNA, 22 bp, Artificial Sequence
  Description of Artificial Sequence Note = Synthetic Construct
tcctttctgaaagacttcgacc

SEQ ID NO: 346 - DNA, 20 bp, Artificial Sequence
  Description of Artificial Sequence Note = Synthetic Construct
cctgtctgactcaatttgct

SEQ ID NO: 347 - DNA, 20 bp, Artificial Sequence
  Description of Artificial Sequence Note = Synthetic Construct
ccgcagggaactctactcac

SEQ ID NO: 348 - DNA, 18 bp, Artificial Sequence
  Description of Artificial Sequence Note = Synthetic Construct
ggatctcgtcccggactc

SEQ ID NO: 349 - DNA, 20 bp, Artificial Sequence
  Description of Artificial Sequence Note = Synthetic Construct
atggatcttggagccagttc

SEQ ID NO: 350 - DNA, 18 bp, Artificial Sequence
  Description of Artificial Sequence Note = Synthetic Construct
acacaaatgagcggacag

SEQ ID NO: 351 - DNA, 20 bp, Artificial Sequence
  Description of Artificial Sequence Note = Synthetic Construct
gtgggaaccatttgcagaag

SEQ ID NO: 352 - DNA, 20 bp, Artificial Sequence
  Description of Artificial Sequence Note = Synthetic Construct
attgcctggcagttcaactc

SEQ ID NO: 353 - DNA, 22 bp, Artificial Sequence
  Description of Artificial Sequence Note = Synthetic Construct
tgatgacaccaatatcacacga

SEQ ID NO: 354 - DNA, 22 bp, Artificial Sequence
  Description of Artificial Sequence Note = Synthetic Construct
ggcttgtaggccttttacttcc

SEQ ID NO: 355 - DNA, 18 bp, Artificial Sequence
  Description of Artificial Sequence Note = Synthetic Construct
ggcctacagccgaggatt

SEQ ID NO: 356 - DNA, 20 bp, Artificial Sequence
  Description of Artificial Sequence Note = Synthetic Construct
gccttatgttcctccagcat

SEQ ID NO: 357 - DNA, 17 bp, Artificial Sequence
  Description of Artificial Sequence Note = Synthetic Construct
gtgtccgttggaaccat

SEQ ID NO: 358 - DNA, 18 bp, Artificial Sequence
  Description of Artificial Sequence Note = Synthetic Construct
ctcagaaggctcatcagt

SEQ ID NO: 359 - DNA, 19 bp, Artificial Sequence
  Description of Artificial Sequence Note = Synthetic Construct
gtttgagctggcttggatg

SEQ ID NO: 360 - DNA, 19 bp, Artificial Sequence
  Description of Artificial Sequence Note = Synthetic Construct
tcttgtcttgcacccactg

SEQ ID NO: 361 - DNA, 23 bp, Artificial Sequence
  Description of Artificial Sequence Note = Synthetic Construct
catgaaatagtgcatagtttgcc

SEQ ID NO: 362 - DNA, 24 bp, Artificial Sequence
  Description of Artificial Sequence Note = Synthetic Construct
ccatcaacattctctttatgaacg

SEQ ID NO: 363 - DNA, 18 bp, Artificial Sequence
  Description of Artificial Sequence Note = Synthetic Construct
gccatcatggtgtttgag

SEQ ID NO: 364 - DNA, 20 bp, Artificial Sequence
  Description of Artificial Sequence Note = Synthetic Construct
gaagtgggacacgtagtaag

SEQ ID NO: 365 - DNA, 21 bp, Artificial Sequence
  Description of Artificial Sequence Note = Synthetic Construct
gctcctaatgtcaaccgagaa

SEQ ID NO: 366 - DNA, 20 bp, Artificial Sequence
  Description of Artificial Sequence Note = Synthetic Construct
agctgcatgtgtggttctgt

SEQ ID NO: 367 - DNA, 22 bp, Artificial Sequence
  Description of Artificial Sequence Note = Synthetic Construct
gagcacaaccacacttacattc

SEQ ID NO: 368 - DNA, 20 bp, Artificial Sequence
  Description of Artificial Sequence Note = Synthetic Construct
gaagttcaagccttgttctc

SEQ ID NO: 369 - DNA, 20 bp, Artificial Sequence
  Description of Artificial Sequence Note = Synthetic Construct
ggagatccgtcaactccaaa

SEQ ID NO: 370 - DNA, 19 bp, Artificial Sequence
  Description of Artificial Sequence Note = Synthetic Construct
agtggacatgcgagtggag

SEQ ID NO: 371 - DNA, 19 bp, Artificial Sequence
  Description of Artificial Sequence Note = Synthetic Construct
cagcaagcgatggcatagt

SEQ ID NO: 372 - DNA, 20 bp, Artificial Sequence
  Description of Artificial Sequence Note = Synthetic Construct
agcgggcttctgtaatctga

SEQ ID NO: 373 - DNA, 19 bp, Artificial Sequence
  Description of Artificial Sequence Note = Synthetic Construct
gatgttcgagtcacagagg

SEQ ID NO: 374 - DNA, 19 bp, Artificial Sequence
  Description of Artificial Sequence Note = Synthetic Construct
gacagctactattcccgtt

SEQ ID NO: 375 - DNA, 17 bp, Artificial Sequence
  Description of Artificial Sequence Note = Synthetic Construct
aatgccaccgaagcctc

SEQ ID NO: 376 - DNA, 19 bp, Artificial Sequence
  Description of Artificial Sequence Note = Synthetic Construct
gcctcagatttcaactcgt

SEQ ID NO: 377 - DNA, 20 bp, Artificial Sequence
  Description of Artificial Sequence Note = Synthetic Construct
tccaggccactgaataacac

SEQ ID NO: 378 - DNA, 20 bp, Artificial Sequence
  Description of Artificial Sequence Note = Synthetic Construct
tttgatgccagttcctcctc

SEQ ID NO: 379 - DNA, 19 bp, Artificial Sequence
  Description of Artificial Sequence Note = Synthetic Construct
aaagattcctgggacctga

SEQ ID NO: 380 - DNA, 22 bp, Artificial Sequence
  Description of Artificial Sequence Note = Synthetic Construct
tggggcagttctgtattacttc

SEQ ID NO: 381 - DNA, 18 bp, Artificial Sequence
  Description of Artificial Sequence Note = Synthetic Construct
ccatgaacgcctcgatgt

SEQ ID NO: 382 - DNA, 20 bp, Artificial Sequence
  Description of Artificial Sequence Note = Synthetic Construct
gagccaccatgtgaggagag

SEQ ID NO: 383 - DNA, 18 bp, Artificial Sequence
  Description of Artificial Sequence Note = Synthetic Construct
cgagatcgccaagatgtt

SEQ ID NO: 384 - DNA, 21 bp, Artificial Sequence
  Description of Artificial Sequence Note = Synthetic Construct
gatggtagagttccagtgatt

SEQ ID NO: 385 - DNA, 21 bp, Artificial Sequence
  Description of Artificial Sequence Note = Synthetic Construct
tggtgacagtggatgattgtg

SEQ ID NO: 386 - DNA, 19 bp, Artificial Sequence
  Description of Artificial Sequence Note = Synthetic Construct
cggtcagatccagggactt

SEQ ID NO: 387 - DNA, 18 bp, Artificial Sequence
  Description of Artificial Sequence Note = Synthetic Construct
ccaggcttctatcgtgac

SEQ ID NO: 388 - DNA, 17 bp, Artificial Sequence
  Description of Artificial Sequence Note = Synthetic Construct
agttggcaggaaggaca

SEQ ID NO: 389 - DNA, 25 bp, Artificial Sequence
  Description of Artificial Sequence Note = Synthetic Construct
tggataatctcaatctgtgtgtttg

SEQ ID NO: 390 - DNA, 20 bp, Artificial Sequence
  Description of Artificial Sequence Note = Synthetic Construct
cgaaacgattgctcaggact

SEQ ID NO: 391 - DNA, 18 bp, Artificial Sequence
  Description of Artificial Sequence Note = Synthetic Construct
gtggcggtttgaccagaa

SEQ ID NO: 392 - DNA, 19 bp, Artificial Sequence
  Description of Artificial Sequence Note = Synthetic Construct
tggtgcacaagacccagac

SEQ ID NO: 393 - DNA, 17 bp, Artificial Sequence
  Description of Artificial Sequence Note = Synthetic Construct
ttcggctggaaggaacc

SEQ ID NO: 394 - DNA, 21 bp, Artificial Sequence
  Description of Artificial Sequence Note = Synthetic Construct
tatgtgagtaagctcggagac

SEQ ID NO: 395 - DNA, 17 bp, Artificial Sequence
  Description of Artificial Sequence Note = Synthetic Construct
tgctggggctggtcctg

SEQ ID NO: 396 - DNA, 17 bp, Artificial Sequence
  Description of Artificial Sequence Note = Synthetic Construct
ggcacggcacactggtt

SEQ ID NO: 397 - DNA, 18 bp, Artificial Sequence
  Description of Artificial Sequence Note = Synthetic Construct
aacttcgctcccaccttc

SEQ ID NO: 398 - DNA, 18 bp, Artificial Sequence
  Description of Artificial Sequence Note = Synthetic Construct
gctggcttccgtttcttg

SEQ ID NO: 399 - DNA, 21 bp, Artificial Sequence
  Description of Artificial Sequence Note = Synthetic Construct
cccaaacaggaacacatagca

SEQ ID NO: 400 - DNA, 19 bp, Artificial Sequence
  Description of Artificial Sequence Note = Synthetic Construct
gagctccatgtgcagaacg

SEQ ID NO: 401 - DNA, 19 bp, Artificial Sequence
  Description of Artificial Sequence Note = Synthetic Construct
agaccgtaggtgggatcag

SEQ ID NO: 402 - DNA, 19 bp, Artificial Sequence
  Description of Artificial Sequence Note = Synthetic Construct
ggtggtggtgacactatgg

SEQ ID NO: 403 - DNA, 22 bp, Artificial Sequence
  Description of Artificial Sequence Note = Synthetic Construct
gccttaacagagcctttatgga

SEQ ID NO: 404 - DNA, 20 bp, Artificial Sequence
  Description of Artificial Sequence Note = Synthetic Construct
cagctatgctgttgtcttca

SEQ ID NO: 405 - DNA, 19 bp, Artificial Sequence
  Description of Artificial Sequence Note = Synthetic Construct
ctcatgcgctgtatgtcca

SEQ ID NO: 406 - DNA, 19 bp, Artificial Sequence
  Description of Artificial Sequence Note = Synthetic Construct
gtccactgccagagacagg

SEQ ID NO: 407 - DNA, 20 bp, Artificial Sequence
  Description of Artificial Sequence Note = Synthetic Construct
gtgaggggtgtcagctcagt

SEQ ID NO: 408 - DNA, 21 bp, Artificial Sequence
  Description of Artificial Sequence Note = Synthetic Construct
cacacagttcactgctccaca

SEQ ID NO: 409 - DNA, 21 bp, Artificial Sequence
  Description of Artificial Sequence Note = Synthetic Construct
ttccactcgatttccaagtga

SEQ ID NO: 410 - DNA, 20 bp, Artificial Sequence
  Description of Artificial Sequence Note = Synthetic Construct
gtggaaatttctcccggaac

SEQ ID NO: 411 - DNA, 23 bp, Artificial Sequence
  Description of Artificial Sequence Note = Synthetic Construct
gtatgctacatgcttcaacatcc

SEQ ID NO: 412 - DNA, 22 bp, Artificial Sequence
  Description of Artificial Sequence Note = Synthetic Construct
aggccattgagagacaaatatc

SEQ ID NO: 413 - DNA, 22 bp, Artificial Sequence
  Description of Artificial Sequence Note = Synthetic Construct
accattatgtctgggtcaaagg

SEQ ID NO: 414 - DNA, 21 bp, Artificial Sequence
  Description of Artificial Sequence Note = Synthetic Construct
ttcttccaaccgatccacttc

SEQ ID NO: 415 - DNA, 19 bp, Artificial Sequence
  Description of Artificial Sequence Note = Synthetic Construct
ctgcaagctgctcaacatc

SEQ ID NO: 416 - DNA, 18 bp, Artificial Sequence
  Description of Artificial Sequence Note = Synthetic Construct
cggtattggtgctctgtc

SEQ ID NO: 417 - DNA, 19 bp, Artificial Sequence
  Description of Artificial Sequence Note = Synthetic Construct
agcctcgaacaattgaaga

SEQ ID NO: 418 - DNA, 20 bp, Artificial Sequence
  Description of Artificial Sequence Note = Synthetic Construct
acacagatgatggagatgtc

SEQ ID NO: 419 - DNA, 21 bp, Artificial Sequence
  Description of Artificial Sequence Note = Synthetic Construct
ttcctactccaatggctcaga

SEQ ID NO: 420 - DNA, 20 bp, Artificial Sequence
  Description of Artificial Sequence Note = Synthetic Construct
agcgtgttccaccatttgta

SEQ ID NO: 421 - DNA, 20 bp, Artificial Sequence
  Description of Artificial Sequence Note = Synthetic Construct
gccatgtgctccaatcatag

SEQ ID NO: 422 - DNA, 19 bp, Artificial Sequence
  Description of Artificial Sequence Note = Synthetic Construct
gccaaacacccaggatctc

SEQ ID NO: 423 - DNA, 19 bp, Artificial Sequence
  Description of Artificial Sequence Note = Synthetic Construct
ggtgacatttctgccactg

SEQ ID NO: 424 - DNA, 17 bp, Artificial Sequence
  Description of Artificial Sequence Note = Synthetic Construct
gtcccgactcttcccat

SEQ ID NO: 425 - DNA, 20 bp, Artificial Sequence
  Description of Artificial Sequence Note = Synthetic Construct
acatttgttggcacacctta

SEQ ID NO: 426 - DNA, 20 bp, Artificial Sequence
  Description of Artificial Sequence Note = Synthetic Construct
attgtaggacatgcgattca

SEQ ID NO: 427 - DNA, 18 bp, Artificial Sequence
  Description of Artificial Sequence Note = Synthetic Construct
gctctgtgtgcccaggat

SEQ ID NO: 428 - DNA, 24 bp, Artificial Sequence
  Description of Artificial Sequence Note = Synthetic Construct
ggaagtcaatctcatcttccagtc

SEQ ID NO: 429 - DNA, 20 bp, Artificial Sequence
  Description of Artificial Sequence Note = Synthetic Construct
attgaccaccgaattccaaa

SEQ ID NO: 430 - DNA, 21 bp, Artificial Sequence
  Description of Artificial Sequence Note = Synthetic Construct
caaagaacccttcatctccaa

SEQ ID NO: 431 - DNA, 21 bp, Artificial Sequence
  Description of Artificial Sequence Note = Synthetic Construct
aaatgccggtcctcagaatac

SEQ ID NO: 432 - DNA, 20 bp, Artificial Sequence
  Description of Artificial Sequence Note = Synthetic Construct
tcctgctttcaggaatactc

SEQ ID NO: 433 - DNA, 21 bp, Artificial Sequence
  Description of Artificial Sequence Note = Synthetic Construct
gattgttgttgttgcaggaga

SEQ ID NO: 434 - DNA, 19 bp, Artificial Sequence
  Description of Artificial Sequence Note = Synthetic Construct
ccttcgtattgtggcattc

SEQ ID NO: 435 - DNA, 27 bp, Artificial Sequence
  Description of Artificial Sequence Note = Synthetic Construct
atcgactgtgtaaacaactagagaaga

SEQ ID NO: 436 - DNA, 25 bp, Artificial Sequence
  Description of Artificial Sequence Note = Synthetic Construct
agtagctacatctccaggttctctg

SEQ ID NO: 437 - DNA, 24 bp, Artificial Sequence
  Description of Artificial Sequence Note = Synthetic Construct
tgaagaaggtctcttttatgctca

SEQ ID NO: 438 - DNA, 23 bp, Artificial Sequence
  Description of Artificial Sequence Note = Synthetic Construct
tgggcaaatactttctttagtgc

SEQ ID NO: 439 - DNA, 23 bp, Artificial Sequence
  Description of Artificial Sequence Note = Synthetic Construct
cccatccatgtgaggaagtataa

SEQ ID NO: 440 - DNA, 22 bp, Artificial Sequence
  Description of Artificial Sequence Note = Synthetic Construct
tgtgaagccagcaatatgtatc

SEQ ID NO: 441 - DNA, 17 bp, Artificial Sequence
  Description of Artificial Sequence Note = Synthetic Construct
aagccacacacaggttc

SEQ ID NO: 442 - DNA, 22 bp, Artificial Sequence
  Description of Artificial Sequence Note = Synthetic Construct
catctccttcacagttaggttg

SEQ ID NO: 443 - DNA, 18 bp, Artificial Sequence
  Description of Artificial Sequence Note = Synthetic Construct
caccttcgcttccagatg

SEQ ID NO: 444 - DNA, 20 bp, Artificial Sequence
  Description of Artificial Sequence Note = Synthetic Construct
gcctattgtctcttgctgtt

SEQ ID NO: 445 - DNA, 20 bp, Artificial Sequence
  Description of Artificial Sequence Note = Synthetic Construct
cctcagatgatgcctatcca

SEQ ID NO: 446 - DNA, 20 bp, Artificial Sequence
  Description of Artificial Sequence Note = Synthetic Construct
gcaggtcaaaactctcaaag

SEQ ID NO: 447 - DNA, 18 bp, Artificial Sequence
  Description of Artificial Sequence Note = Synthetic Construct
aactcgcaaacgcaacct

SEQ ID NO: 448 - DNA, 21 bp, Artificial Sequence
  Description of Artificial Sequence Note = Synthetic Construct
tcttggaagagtccacaatcc

SEQ ID NO: 449 - DNA, 18 bp, Artificial Sequence
  Description of Artificial Sequence Note = Synthetic Construct
caaggtttcatccgcctc

SEQ ID NO: 450 - DNA, 17 bp, Artificial Sequence
  Description of Artificial Sequence Note = Synthetic Construct
tcatcactgctcacgct

SEQ ID NO: 451 - DNA, 18 bp, Artificial Sequence
  Description of Artificial Sequence Note = Synthetic Construct
tgccgcagaactcacttg

SEQ ID NO: 452 - DNA, 19 bp, Artificial Sequence
  Description of Artificial Sequence Note = Synthetic Construct
catttgccgtccttcatcg

SEQ ID NO: 453 - DNA, 21 bp, Artificial Sequence
  Description of Artificial Sequence Note = Synthetic Construct
ccaatggggagatacagaagg

SEQ ID NO: 454 - DNA, 18 bp, Artificial Sequence
  Description of Artificial Sequence Note = Synthetic Construct
cactggaggcggatctca

SEQ ID NO: 455 - DNA, 18 bp, Artificial Sequence
  Description of Artificial Sequence Note = Synthetic Construct
ccattacggcgatcaagg

SEQ ID NO: 456 - DNA, 20 bp, Artificial Sequence
  Description of Artificial Sequence Note = Synthetic Construct
cccacaggctctaggtcact

SEQ ID NO: 457 - DNA, 21 bp, Artificial Sequence
  Description of Artificial Sequence Note = Synthetic Construct
gacaaagacaggaggaaagag

SEQ ID NO: 458 - DNA, 20 bp, Artificial Sequence
  Description of Artificial Sequence Note = Synthetic Construct
tccctgtgaagtggctatta

SEQ ID NO: 459 - DNA, 20 bp, Artificial Sequence
  Description of Artificial Sequence Note = Synthetic Construct
tttaagagggcaatggaagg

SEQ ID NO: 460 - DNA, 21 bp, Artificial Sequence
  Description of Artificial Sequence Note = Synthetic Construct
cggattttatcaacgatgcag

SEQ ID NO: 461 - DNA, 18 bp, Artificial Sequence
  Description of Artificial Sequence Note = Synthetic Construct
tacctgaaccggcacctg

SEQ ID NO: 462 - DNA, 20 bp, Artificial Sequence
  Description of Artificial Sequence Note = Synthetic Construct
gccgtacagttccacaaagg

SEQ ID NO: 463 - DNA, 20 bp, Artificial Sequence
  Description of Artificial Sequence Note = Synthetic Construct
tgctctccttcatcaatcca

SEQ ID NO: 464 - DNA, 20 bp, Artificial Sequence
  Description of Artificial Sequence Note = Synthetic Construct
tggggttggagataaacctg

SEQ ID NO: 465 - DNA, 18 bp, Artificial Sequence
  Description of Artificial Sequence Note = Synthetic Construct
gaagatcgtcgccacctg

SEQ ID NO: 466 - DNA, 20 bp, Artificial Sequence
  Description of Artificial Sequence Note = Synthetic Construct
gacctcctcctcgcacttct

SEQ ID NO: 467 - DNA, 18 bp, Artificial Sequence
  Description of Artificial Sequence Note = Synthetic Construct
ggccaaaatcgacaggac

SEQ ID NO: 468 - DNA, 19 bp, Artificial Sequence
  Description of Artificial Sequence Note = Synthetic Construct
gggtctgcacagactgcat

SEQ ID NO: 469 - DNA, 19 bp, Artificial Sequence
  Description of Artificial Sequence Note = Synthetic Construct
ccaccaaagtcacgctgaa

SEQ ID NO: 470 - DNA, 19 bp, Artificial Sequence
  Description of Artificial Sequence Note = Synthetic Construct
tgcttggattccagaaacg

SEQ ID NO: 471 - DNA, 25 bp, Artificial Sequence
  Description of Artificial Sequence Note = Synthetic Construct
acacagaatctatacccaccagagt

SEQ ID NO: 472 - DNA, 20 bp, Artificial Sequence
  Description of Artificial Sequence Note = Synthetic Construct
atcaactcccaaacggtcac

SEQ ID NO: 473 - DNA, 18 bp, Artificial Sequence
  Description of Artificial Sequence Note = Synthetic Construct
tcgaccacgtcaagaagc

SEQ ID NO: 474 - DNA, 20 bp, Artificial Sequence
  Description of Artificial Sequence Note = Synthetic Construct
gttcttagcatccttgaggg

SEQ ID NO: 475 - DNA, 18 bp, Artificial Sequence
  Description of Artificial Sequence Note = Synthetic Construct
aggcgaacacacaacgtc

SEQ ID NO: 476 - DNA, 18 bp, Artificial Sequence
  Description of Artificial Sequence Note = Synthetic Construct
tctggtcacgcagggcaa

SEQ ID NO: 477 - DNA, 22 bp, Artificial Sequence
  Description of Artificial Sequence Note = Synthetic Construct
gttggaccagtcaacatctctg

SEQ ID NO: 478 - DNA, 18 bp, Artificial Sequence
  Description of Artificial Sequence Note = Synthetic Construct
gccatagccactgccact

SEQ ID NO: 479 - DNA, 19 bp, Artificial Sequence
  Description of Artificial Sequence Note = Synthetic Construct
acctcaccctgtaccagtc

SEQ ID NO: 480 - DNA, 19 bp, Artificial Sequence
  Description of Artificial Sequence Note = Synthetic Construct
ctgctggtccttcccatag

SEQ ID NO: 481 - DNA, 20 bp, Artificial Sequence
  Description of Artificial Sequence Note = Synthetic Construct
ccggagctgcctatgtaatc

SEQ ID NO: 482 - DNA, 20 bp, Artificial Sequence
  Description of Artificial Sequence Note = Synthetic Construct
cagaggcccatgaacacatc

SEQ ID NO: 483 - DNA, 18 bp, Artificial Sequence
  Description of Artificial Sequence Note = Synthetic Construct
cattcgtgctatccctgg

SEQ ID NO: 484 - DNA, 20 bp, Artificial Sequence
  Description of Artificial Sequence Note = Synthetic Construct
tgtagaaatctgcgtatgcc

SEQ ID NO: 485 - DNA, 18 bp, Artificial Sequence
  Description of Artificial Sequence Note = Synthetic Construct
ctgaccctgtctctgtgt

SEQ ID NO: 486 - DNA, 20 bp, Artificial Sequence
  Description of Artificial Sequence Note = Synthetic Construct
gttcaaaccttcctcttcgt

SEQ ID NO: 487 - DNA, 18 bp, Artificial Sequence
  Description of Artificial Sequence Note = Synthetic Construct
caacatggacaaaccacg

SEQ ID NO: 488 - DNA, 17 bp, Artificial Sequence
  Description of Artificial Sequence Note = Synthetic Construct
cctctcctcatcggtct

SEQ ID NO: 489 - DNA, 19 bp, Artificial Sequence
  Description of Artificial Sequence Note = Synthetic Construct
attcccagagcccacaata

SEQ ID NO: 490 - DNA, 20 bp, Artificial Sequence
  Description of Artificial Sequence Note = Synthetic Construct
atccactggcagtacagaag

SEQ ID NO: 491 - DNA, 21 bp, Artificial Sequence
  Description of Artificial Sequence Note = Synthetic Construct
actcagtacaagaaagaaccg

SEQ ID NO: 492 - DNA, 17 bp, Artificial Sequence
  Description of Artificial Sequence Note = Synthetic Construct
gaggagatgaccttgcc

SEQ ID NO: 493 - DNA, 24 bp, Artificial Sequence
  Description of Artificial Sequence Note = Synthetic Construct
gacaaggagaatcaaaagatcagc

SEQ ID NO: 494 - DNA, 20 bp, Artificial Sequence
  Description of Artificial Sequence Note = Synthetic Construct
actgtctgggtccatggcta

SEQ ID NO: 495 - DNA, 17 bp, Artificial Sequence
  Description of Artificial Sequence Note = Synthetic Construct
caagctagacctggact

SEQ ID NO: 496 - DNA, 17 bp, Artificial Sequence
  Description of Artificial Sequence Note = Synthetic Construct
gcattgctctgggtgat

SEQ ID NO: 497 - DNA, 18 bp, Artificial Sequence
  Description of Artificial Sequence Note = Synthetic Construct
aggctctgcttctgtacc

SEQ ID NO: 498 - DNA, 18 bp, Artificial Sequence
  Description of Artificial Sequence Note = Synthetic Construct
cggacgcactttcttctc

SEQ ID NO: 499 - DNA, 17 bp, Artificial Sequence
  Description of Artificial Sequence Note = Synthetic Construct
gtggcagcagatcacaa

SEQ ID NO: 500 - DNA, 18 bp, Artificial Sequence
  Description of Artificial Sequence Note = Synthetic Construct
ggatttcgtggtgggttc

SEQ ID NO: 501 - DNA, 21 bp, Artificial Sequence
  Description of Artificial Sequence Note = Synthetic Construct
caacatgccaattgagtgaaa

SEQ ID NO: 502 - DNA, 21 bp, Artificial Sequence
  Description of Artificial Sequence Note = Synthetic Construct
acttgggccttaaacttcacc

SEQ ID NO: 503 - DNA, 20 bp, Artificial Sequence
  Description of Artificial Sequence Note = Synthetic Construct
caaacgtgtgttctggaagg

SEQ ID NO: 504 - DNA, 22 bp, Artificial Sequence
  Description of Artificial Sequence Note = Synthetic Construct
acagctctttagcatttgtgga

SEQ ID NO: 505 - DNA, 19 bp, Artificial Sequence
  Description of Artificial Sequence Note = Synthetic Construct
ctttcgcctgagcctattt

SEQ ID NO: 506 - DNA, 18 bp, Artificial Sequence
  Description of Artificial Sequence Note = Synthetic Construct
gggcacatccagatgttt

SEQ ID NO: 507 - DNA, 19 bp, Artificial Sequence
  Description of Artificial Sequence Note = Synthetic Construct
gtctctggtaatgcacact

SEQ ID NO: 508 - DNA, 18 bp, Artificial Sequence
  Description of Artificial Sequence Note = Synthetic Construct
ctgatggttgaggctgtt

SEQ ID NO: 509 - DNA, 20 bp, Artificial Sequence
  Description of Artificial Sequence Note = Synthetic Construct
ctacgaccgcaaggactacg

SEQ ID NO: 510 - DNA, 18 bp, Artificial Sequence
  Description of Artificial Sequence Note = Synthetic Construct
gtggtggtgttggtggtg

SEQ ID NO: 511 - DNA, 19 bp, Artificial Sequence
  Description of Artificial Sequence Note = Synthetic Construct
atcggcagcaacattgtca

SEQ ID NO: 512 - DNA, 19 bp, Artificial Sequence
  Description of Artificial Sequence Note = Synthetic Construct
cacgcagttcatccatagg

SEQ ID NO: 513 - DNA, 19 bp, Artificial Sequence
  Description of Artificial Sequence Note = Synthetic Construct
caaggaaacaggtctctgg

SEQ ID NO: 514 - DNA, 17 bp, Artificial Sequence
  Description of Artificial Sequence Note = Synthetic Construct
gcaggcttctcttcacg

SEQ ID NO: 515 - DNA, 17 bp, Artificial Sequence
  Description of Artificial Sequence Note = Synthetic Construct
tgccatgttgagcctga

SEQ ID NO: 516 - DNA, 17 bp, Artificial Sequence
  Description of Artificial Sequence Note = Synthetic Construct
caaggggactcggtaga

SEQ ID NO: 517 - DNA, 18 bp, Artificial Sequence
  Description of Artificial Sequence Note = Synthetic Construct
tggattctgggctccaag

SEQ ID NO: 518 - DNA, 18 bp, Artificial Sequence
  Description of Artificial Sequence Note = Synthetic Construct
caactctcccgttgagtc

SEQ ID NO: 519 - DNA, 20 bp, Artificial Sequence
  Description of Artificial Sequence Note = Synthetic Construct
cgcagtcatccagagatgtg

SEQ ID NO: 520 - DNA, 19 bp, Artificial Sequence
  Description of Artificial Sequence Note = Synthetic Construct
cgtgcacatccatgacctt

SEQ ID NO: 521 - DNA, 18 bp, Artificial Sequence
  Description of Artificial Sequence Note = Synthetic Construct
gtcatggccgagcagaac

SEQ ID NO: 522 - DNA, 20 bp, Artificial Sequence
  Description of Artificial Sequence Note = Synthetic Construct
ccggttcaattcttcagtcc

SEQ ID NO: 523 - DNA, 19 bp, Artificial Sequence
  Description of Artificial Sequence Note = Synthetic Construct
gatgaaccggaacatcagc

SEQ ID NO: 524 - DNA, 18 bp, Artificial Sequence
  Description of Artificial Sequence Note = Synthetic Construct
ctccagggaagccctctg

SEQ ID NO: 525 - DNA, 17 bp, Artificial Sequence
  Description of Artificial Sequence Note = Synthetic Construct
agcactttgtcacccag

SEQ ID NO: 526 - DNA, 18 bp, Artificial Sequence
  Description of Artificial Sequence Note = Synthetic Construct
gccacaccaacttcaacc

SEQ ID NO: 527 - DNA, 22 bp, Artificial Sequence
  Description of Artificial Sequence Note = Synthetic Construct
tgagattgaggatgaagctgag

SEQ ID NO: 528 - DNA, 20 bp, Artificial Sequence
  Description of Artificial Sequence Note = Synthetic Construct
cattggtggtgaagctcttg

SEQ ID NO: 529 - DNA, 19 bp, Artificial Sequence
  Description of Artificial Sequence Note = Synthetic Construct
agtgtgtgcccactgagga

SEQ ID NO: 530 - DNA, 20 bp, Artificial Sequence
  Description of Artificial Sequence Note = Synthetic Construct
ggtgaggtttgatccgcata

SEQ ID NO: 531 - DNA, 22 bp, Artificial Sequence
  Description of Artificial Sequence Note = Synthetic Construct
caacattaatgatcggagtgct

SEQ ID NO: 532 - DNA, 19 bp, Artificial Sequence
  Description of Artificial Sequence Note = Synthetic Construct
ctcctccagagccatagcc

SEQ ID NO: 533 - DNA, 21 bp, Artificial Sequence
  Description of Artificial Sequence Note = Synthetic Construct
cagtattccaaccctcctgtg

SEQ ID NO: 534 - DNA, 19 bp, Artificial Sequence
  Description of Artificial Sequence Note = Synthetic Construct
gttctcctgcaccctggtt

(19) United States
(12) Patent Application Publication
(10) Pub. No.: US 2017/0044618 A1
(43) Pub. Date: Feb. 16, 2017

(54) METHODS AND COMPOSITIONS INVOLVING INTRINSIC GENES

(71) Applicants: Washington University, St. Louis, MO (US); The University of North Carolina at Chapel Hill, Chapel Hill, NC (US); University of Utah Research Foundation, Salt Lake City, UT (US)

(72) Inventors: Matthew J. ELLIS, St. Louis, MO (US); Philip S. BERNARD, Salt Lake City, UT (US); Robert A. PALAIS, Salt Lake City, UT (US); Charles M. PEROU, Carrboro, NC (US)

(21) Appl. No.: 15/043,022

(22) Filed: Feb. 12, 2016

Related U.S. Application Data
(63) Continuation of application No. 13/959,575, filed on Aug. 5, 2013, now abandoned.
Application No. 13/959,575 is a continuation of application No. 12/094,898, filed on Mar. 13, 2009, now abandoned.
(60) Provisional application No. 60/739,155, filed on Nov. 23, 2005.

(51) Int. Cl. C12Q 1/68

(52) U.S. Cl. CPC C12Q 1/6886; C12Q 2600/158; C12Q 2600/112; C12Q 2600/118

(57) ABSTRACT

Disclosed are compositions and methods related intrinsic gene sets and methods and compositions related to detecting and classifying cancer.

### CROSS-REFERENCE TO RELATED APPLICATIONS

This application is a continuation of U.S. patent application Ser. No. 13/959,575, filed Aug. 5, 2013. U.S. Ser. No. 13/959,575 is a continuation of U.S. patent application Ser. No. 12/094,898, filed Mar. 13, 2009. U.S. Ser. No. 12/094,898 is a National Stage Application, filed under 35 U.S.C. §371 of International Application No. PCT/US2006/044737, filed Nov. 17, 2006. PCT/US2006/044737 claims priority to, and the benefit of, U.S. Provisional Application Ser. No. U.S. 60/739,155, filed Nov. 23, 2005. The contents of the aforementioned applications are incorporated herein by reference in their entireties.

### GOVERNMENT SUPPORT

This work was supported in part by the National Institutes of Health (CA097769, CA114722, and CA058223). The United States Government may have certain rights in the inventions disclosed herein.

### INCORPORATION-BY-REFERENCE OF SEQUENCE LISTING

The instant application contains a Sequence Listing which has been submitted in ASCII format via EFS-Web and is hereby incorporated by reference in its entirety. Said ASCII copy, created on Feb. 12, 2016, is named NATE-701C02US_ST25.txt and is 126,940 bytes in size.

### BACKGROUND

2. A major challenge for microarray studies, especially those with clinical implications, is validation (Ioannidis 2005; Jenssen and Hovig 2005; Michiels et al. 2005). Due to the practical considerations of cost and accessing large numbers of fresh samples with associated clinical information, very few microarray studies have analyzed enough samples to allow the findings to be extended to the general population. Furthermore, it has been difficult to combine and/or validate results from independent laboratories due to differences in sample preparation, patient demographics and the microarray platforms used. An accepted method for validation is to derive a prognostic gene set from a “training set” and then apply it to a “test set” that was not used in any way, to derive the prognostic gene set (Simon et al. 2003); the “purest” test sets have also been suggested to be comprised of samples not contained in the training set and not generated by the primary investigators (Ioannidis 2005). What is needed in the art is a new breast tumor intrinsic gene list that identifies new and important biological features of breast tumors and validates this predictor using a true test set.

### SUMMARY

Described herein is a method of diagnosing cancer, the method comprising comparing expression levels of a combination of genes from Table 21 to test nucleic acids wherein specific expression patterns of the test nucleic acids indicates a cancerous state.

Also, disclosed is a method of quantitating level of expression of a test nucleic acid comprising: a) comparing gene expression levels of a combination of genes from Table 21 to test nucleic acids corresponding to the same combination of genes; and b) quantitating level of expression of the test nucleic acid.

Also disclosed is a method for determining prognosis based on the expression patterns in a subject diagnosed with cancer comprising: a) comparing expression levels of a combination of genes from Table 21 to test nucleic acids corresponding to the same combination of genes; and b) quantitating level of expression of the test nucleic acid.

Disclosed is a method of classifying cancer in a subject, comprising: a) identifying intrinsic genes of the subject to be used to classify the cancer; b) obtaining a sample from the subject; c) amplifying and detecting levels of intrinsic genes in the subject; and d) classifying cancer or subject based upon results of step c.

Also disclosed is a method of diagnosing cancer in a subject the method comprising: a) amplifying and detecting intrinsic genes; and b) diagnosing cancer based on expression levels of the gene within the subject.

Disclosed herein is a method of deriving a minimal intrinsic gene set for making biological classifications of cancer comprising: a) collecting data from multiple samples from the same individual to identify potential intrinsic classifier genes; b) weighting intrinsic classifier genes of multiple individuals identified using the method of step a relative to each other and forming classification clusters; c) estimating the number of clusters formed in step b) and assigning individual samples to classification clusters; d) identifying genes that optimally distinguish the samples in the assigned groups of step c); e) performing iterative cross-validation with a nearest centroid classifier and overlapping gene sets of various sizes using the genes identified in step d); and f) choosing a gene set which provides the highest class prediction accuracy when compared to the classifications made in step b).

Also disclosed is a method of assigning a sample to an intrinsic subtype, comprising a) creating an intrinsic subtype average profile (centroid) for each subtype; b) individually comparing a new sample to each centroid; and c) assigning the new sample to the centroid that is most similar to the expression profile of new sample.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the expression levels for the five genes shown by tissue sample. Top: raw data. Bottom: log-scale.

FIG. 2 shows the expression levels of the 10 genes shown by sample and tissue type. Vandesompele data set in log-scale.

FIG. 3 shows the mean squared error (MSE) of each gene by tissue-type. The sign is determined by the direction of the bias. The MSE is broken down into the contributing components of the squared bias (Biaŝ2) and the variance (Sigmâ2). Vandesompele data set.

FIG. 4 shows two-way hierarchical clustering of microarray data for the same samples assayed by qRT-PCR. Samples were classified based on the expression of 402 “intrinsic” genes defined in Sorlie et al. 2003. The expression level for each gene is shown relative to the median expression of that gene across all the samples with high expression represented by red and low expression represented by green. Genes with median expression are black and missing values are gray. The sample-associated dendrogram shows the same classes seen by qRT-PCR (FIG. 5). Samples are grouped into Luminal, HER2+/ER−, Normal-like, and Basal-like subtypes. Overall, 114/123 (93%) primary breast samples classified the same between microarray and qRT-PCR.

FIG. 5 shows two-way hierarchical clustering of real-time qRT-PCR data from 126 unique samples. The sample-associated dendrogram (5A) shows the same classes seen by microarray. Samples are grouped into Luminal (blue), HER2+/ER− (pink), Normal-like (green), and Basal-like (red) subtypes. The expression level for each gene is shown relative to the median expression of that gene across all the samples with high expression represented by red and low expression represented by green. Genes with median expression are black and missing values are gray. A minimal set of 37 “intrinsic” genes (5B) was used to classify tumors into their primary “intrinsic” subtypes. The “intrinsic” gene set was supplemented using PgR and EGFR (5C), and proliferation genes (5D). The genes in 1C and ID were clustered separately in order to determine agreement between the minimal 37 qRT-PCR “intrinsic” set (5A) and the larger 402 microarray “intrinsic” set.

FIG. 6 shows Receiver Operator Curves. The agreement between immunohistochemistry (IHC) and gene expression is shown for ER (6A), PR (6B), and HER2 (6C) using ROC. A cut-off for relative gene copy number was selected by minimizing the sum of the observed false positive and false negative errors. The sensitivity and specificity of the resulting classification rule were estimated via bootstrap adjustment for optimism. Since many biomarkers having concordant expression and can serve as surrogates for one another, we tested the accuracy of using GATA3 and GRB7 as surrogates (dotted lines) for calling ER and HER2 protein status, respectively. There was overall good agreement between gene expression and IHC status for ER and PR, but poor agreement between gene expression and IHC status for HER2. The surrogate markers had similar accuracy to the actual markers for predicting HiC status.

FIG. 7 shows outcome for “intrinsic” subtypes. Kaplan-Meier plots showing relapse free survival (RFS) and overall survival (OS) for patients with Luminal tumors compared to those with HER2+/ER− or Basal-like tumors. Patients with Luminal tumors showed significantly better outcomes for RFS (3A) and OS (3B) compared to HER2+/ER−(RFS: ρ=0.023; OS: p=0.003) and Basal-like (RFS: ρ=0.065; OS: p=0.002) tumors. Classifications were made from real-time qRT-PCR data using the minimal 37 “intrinsic” gene list. Pairwise log-rank tests were used to test for equality of the hazard functions among the intrinsic classes. Tumors in the Normal Breast-like subtype were excluded from the analyses since this class maybe artificially created from having a sample comprised primarily of normal cells.

FIG. 8 shows grade and proliferation as predictors of relapse free survival. Kaplan-Meier plots are shown for grade (8A) and the proliferation genes (8B) using Cox regression analysis. The analysis for the proliferation genes was performed on continuous expression data, although the plots are shown in tertiles. The proliferation index (log average of the 14 proliferation genes) has significant predictive value for outcome, even after correcting for other clinical parameters important for survival. Furthermore, when we include both grade and the proliferation index (and stage) in a model for RFS, we find that the proliferation index is the superior predictor (Grade p=0.51; Proliferation index p=0.047).

FIG. 9 shows co-clustering of real-time qRT-PCR and microarray data using 50 genes and 252 samples. The relative copy number (qRT-PCR) and R/G ratio (microarray) for each gene was Iog2 transformed and combined into a single dataset using distance weighted discrimination. Two-way hierarchical clustering was performed on the combined dataset using Spearman correlation and average linkage. The sample associated dendrogram (5A) shows the same classes as seen in FIG. 1. Samples are classified as Basal-like (red), HER2+/ER−, Luminal, and Normal-like. The expression level for each gene is shown relative to the median expression of that gene across all the samples with overexpressed genes and underexpressed genes, as well as average expression. The gene associated dendrogram (5B) shows that the Luminal tumors and Basal-like tumors differentially express estrogen associated genes (cluster 1); as well as basal keratins (KRT 5 and 17), inflammatory response genes (CX3CL1 and SLPI), and genes in the Wnt pathway (FZD7) (cluster 3). The main distinguishers of the HER2+/ER− group are low expression of genes in cluster 1 and high expression of genes on the 1/q12 amplicon (ERBB2 and GRB7) (cluster 4). The proliferation genes (cluster 2) have high expression in the ER negative tumors (Basal-like and HER2+/ER−) and low expression in ER positive (Luminal) and Normal-like samples.

FIG. 10 shows a flow chart of the steps of deriving minimal intrinsic gene sets for making biological classifications of breast cancer.

FIG. 11 shows an overview and flow of the data sets used and analyses performed.

FIG. 12 shows a hierarchical cluster analysis of the training set using the Intrinsic/UNC gene set. 146 microarrays, representing 105 tumors and 9 normal breast samples were analyzed using the 1300 gene Intrinsic/UNC gene set. A) Overview of the complete cluster diagram (the full cluster diagram can be found as Supplemental FIG. 1). B) Experimental sample associated dendrogram. The 26 paired samples used for the intrinsic analysis are identified by the black bars. C) Luminal/ER+ gene expression cluster with GATA3-regulated genes shown in pink. D) HER2 and GRB7 containing expression cluster. E) Basal epithelial enriched expression cluster. F) Proliferation associated expression cluster. The genes in red are mentioned in the text. The Single Sample Predictor/SSP was applied back onto this training data set with the individual sample classifications identified using colored squares (Pink=HER2+/ER−, Red=Basal-like, Dark Blue=Luminal A, Light Blue=Luminal B, and Green=Normal Breast-like).

FIG. 13 shows Androgen Receptor (AR) immunohistochemistry on human breast tumors. A) AR staining on the HER2+/ER− subtype tumor BR00-0284. B) AR staining on the HER2+/ER− subtype tumor PB455 showing nuclear localization. C) AR staining on the Luminal subtype tumor BR01-0246. D) Lack of AR staining on the Basal-like subtype tumor BR97-0137. The magnification is approximately 200×.

FIG. 14 shows hierarchical cluster analysis the combined test set of 311 tumors and 4 normal breast samples analyzed using the Intrinsic/UNC gene set reduced to 306 genes. A) Overview of the complete cluster diagram. B) Experimental sample associated dendrogram. C) Luminal/ER+ gene expression cluster with GATA3-regulated genes in pink text. D) HER2 and GRB7 containing expression cluster. E) Interferon-regulated cluster containing STAT1. F) Basal epithelial enriched cluster. G) proliferation cluster.

FIG. 15 shows univariate Kaplan-Meier survival plots using RFS as the endpoint, for the common clinical parameters present within the combined test set of 311 tumors. Survival plots for A) ER status, B) node status, C) grade, and D) tumor size.

FIG. 16 shows univariate Kaplan-Meier survival plots for intrinsic subtype analyses. A) Relapse-free survival for the 105 patients/tumors training set classified using hierarchical clustering and complete 1300 gene the Intrinsic/UNC list. B) Relapse-free survival for the 315 sample combined test set analyzed using the Intrinsic/UNC list reduced to 306 genes. C) Survival analysis of the 60 adjuvant tamoxifen-treated patients from the Ma et al. 2004 study who were classified as either LumA, LumB or Normal Breast-like using the Single Sample Predictor. D) Survival analysis of the 96 local treatment only (i.e. surgery alone) test set patients taken from Chang et al. 2005, which were classified using the Single Sample Predictor. E) Survival analysis of a second pure test set of 45 patients treated with adjuvant tamoxifen and classified using the Single Sample Predictor. F) Relapse-free survival for the 105 patients/tumors training set, and classified using the Single Sample Predictor. All p-values were based on a log-rank test.

FIG. 17 shows grade and proliferation as predictors of relapse free survival. A Cox regression model was used to determine probability of relapse over time. Kaplan-Meier curves show time to event given different grades and levels of proliferation. Grade was scored as low (green), medium (red) or high (blue). The proliferation score was based on continuous expression data and is shown as textiles that correspond to low (green), medium (red), and high (blue) levels of expression. The proliferation meta-gene (log 2 average of the 14 proliferation genes) showed significant value in predicting relapse, even after correcting for other clinical parameters important for survival (Table 1). Furthermore, when both grade and proliferation were used in a model for RFS, it was found that the proliferation meta-gene is the better predictor (Grade p=0.51; Proliferation index p=0.047).

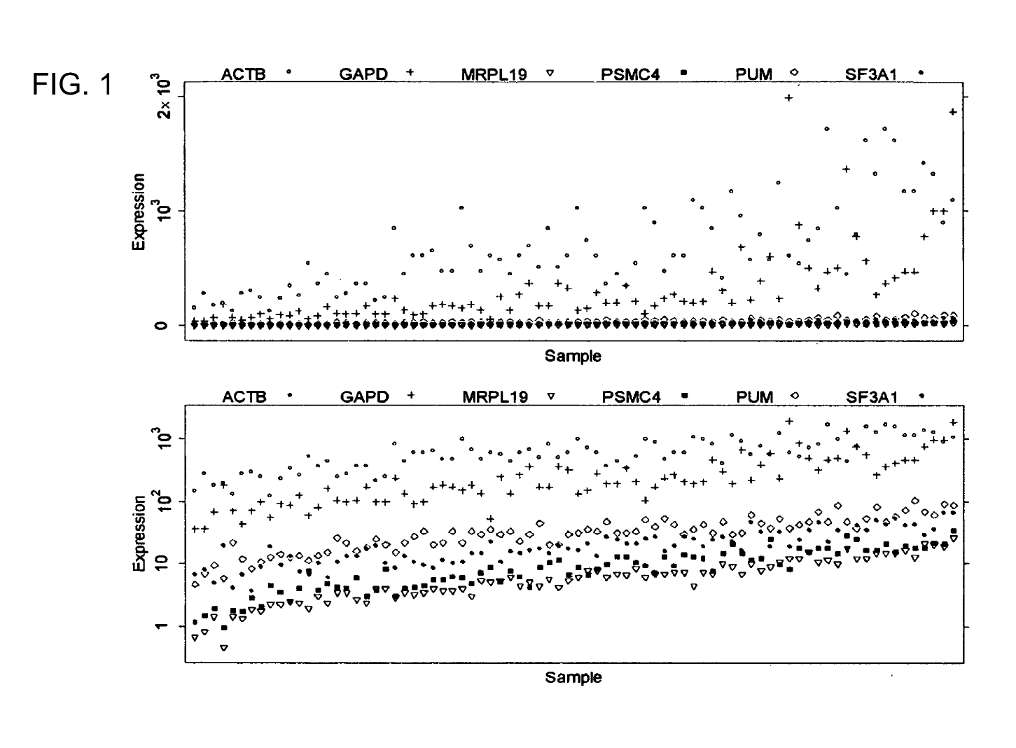

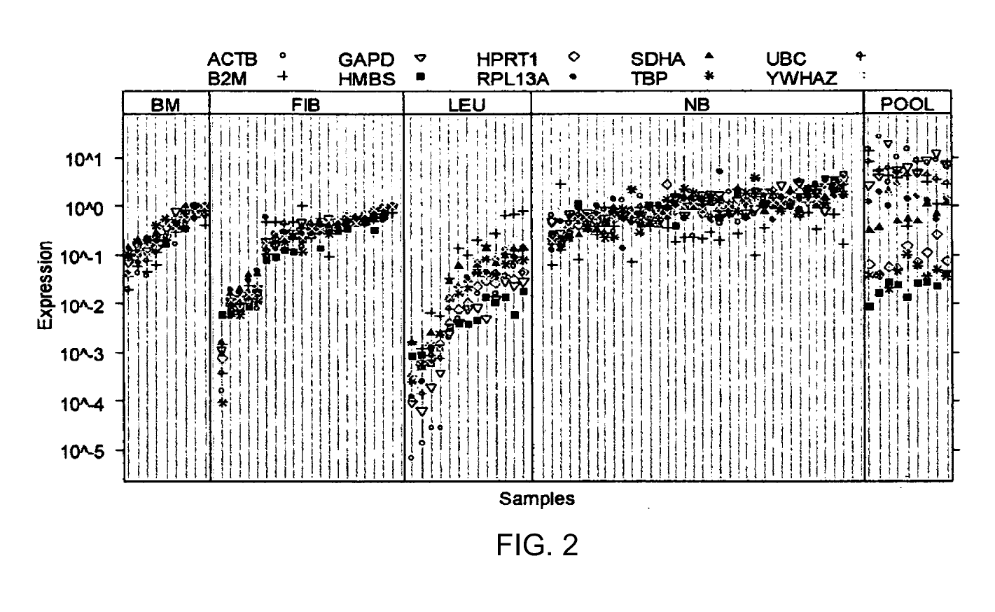

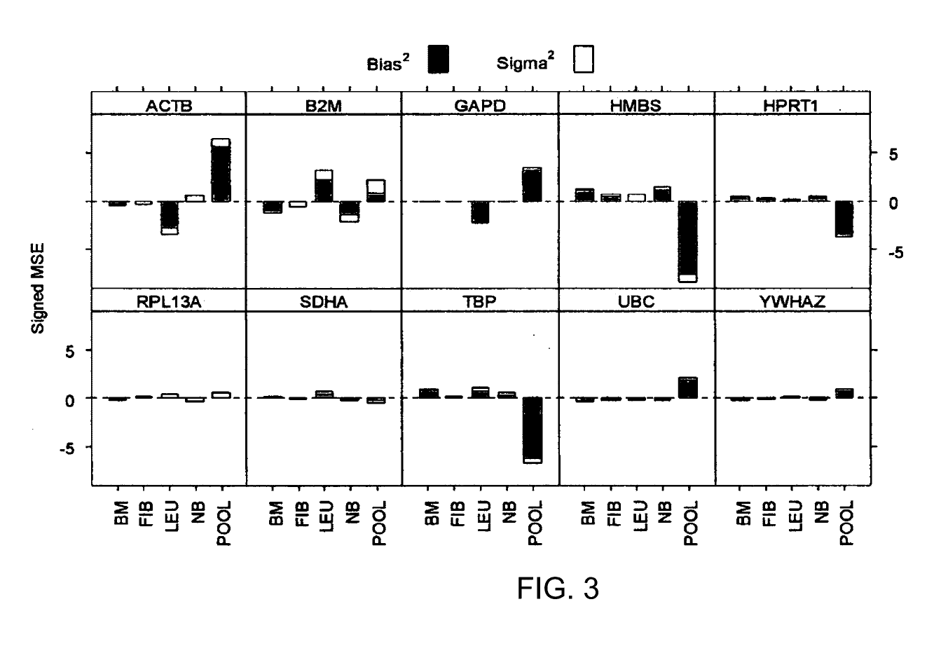

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

By “specific expression pattern” is meant an elevation or reduction of expression of given genes when compared with a control or a standard. One of ordinary skill in the art is capable of identifying and measuring the expression of gene patterns of genes related to the methods disclosed herein.

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout the description and claims of this specification, the word “comprise” and variations of the word, such as “comprising” and “comprises,” means “including but not limited to,” and is not intended to exclude, for example, other additives, components, integers or steps.

The term “cell” as used herein also refers to individual cells, cell lines, or cultures derived from such cells. A “culture” refers to a composition comprising isolated cells of the same or a different type.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, an Y are present at a weight ratio of 2:5, and are present in such ratio regardless of whether additional components are contained in the compound.

A weight percent of a component, unless specifically stated to the contrary, is based on the total weight of the formulation or composition in which the component is included.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

### B. COMPOSITIONS AND METHODS

Disclosed herein are methods and compositions for deriving a minimal intrinsic gene set for making biological classifications of cancer. Also disclosed are methods of using intrinsic genes in a real-time qRT-PCR assay for cancer classification, prognosis and/or treatment. Described herein are several algorithms for use in combination in order to generate a statistically validated minimal gene set that makes biological classifications of cancers. While the methods disclosed herein are generally useful with any type of cancer, breast cancer is specifically used as an example herein. Below follows a list of specific cancers that are useful with the methods disclosed herein, and the example of breast cancer is not intended to be limiting, but rather exemplary. The samples disclosed herein can be obtained from a variety of sources, including fresh tissue, fresh-frozen samples, or formalin-fixed paraffin-embedded samples.

The methodology described herein can be used to make a classification that distinguishes 2 or more intrinsic subtypes of breast cancer. The intrinsic subtypes can be designated as Luminal (and classes therein), HER2/ER− (and classes therein), Basal (and classes therein), Normal-like (and classes therein). The steps for finding the minimal intrinsic gene set for making subtype (and class) distinctions are as follows.

The first step is to use microarray data from biological replicates from the same patient to find intrinsic classifier genes. For example, a data set of tumors and normal breast samples can be used. In one embodiment, these data sets can comprise paired biological replicates to identify the intrinsic gene set. This is described, for example, in Perou et al. (2000), which is herein incorporated by reference in its entirety for its teaching regarding finding intrinsic classifier genes. In Perou et al., the molecular portraits revealed in the patterns of gene expression not only uncovered similarities and differences among the tumors, but also point to a biological interpretation. Variation in growth rate, in the activity of specific signalling pathways, and in the cellular composition of the tumors were all reflected in the corresponding variation in the expression of specific subsets of genes.

In the second step of the method disclosed herein, hierarchical cluster microarray data was obtained using an intrinsic gene set. Here, data can be combined from different microarray platforms for clustering using methods described in Example 2. Specifically, the “intrinsic gene set” from the first step (above) is tested on new tumors and normal breast samples after combining different datasets (such as cross platform analyses) and common genes/elements are hierarchically clustered. For example, a two-way average linkage hierarchical cluster analysis can be performed using a centered Pearson correlation metric and the program “Cluster” (Eisen et al. 1998), with the data being displayed relative to the median expression for each gene (i.e. median centering of the rows/genes).

In the third step, the number of clusters formed in the microarray dataset is estimated, and samples/tumors are assigned to clusters based on the sample-associated dendrogram groupings. In other words, the “test set” is used as a training set to create subtype centroids based upon the expression of the common intrinsic genes. New samples are assigned to the subtype corresponding to the nearest centroid when using Spearman correlation values.

In the fourth step, genes are found that optimally distinguish the samples in the assigned groups using the ratio of between-group to within-group sums of squares (the entire microarray dataset is used in this analysis). An example of this can be found in Chung et al, Cancer Cell 2004, herein incorporated by reference in its entirety for its teaching concerning identification of genes that optimally distinguish samples.

In the fifth step, iterative cycles of 10-fold cross-validation are performed with a nearest centroid classifier and overlapping gene sets of varying sizes. In other words, each gene and gene set are ranked based upon the metric from step four above, and various overlapping and every increasing sized genes lists are used in a 10-fold cross validation.

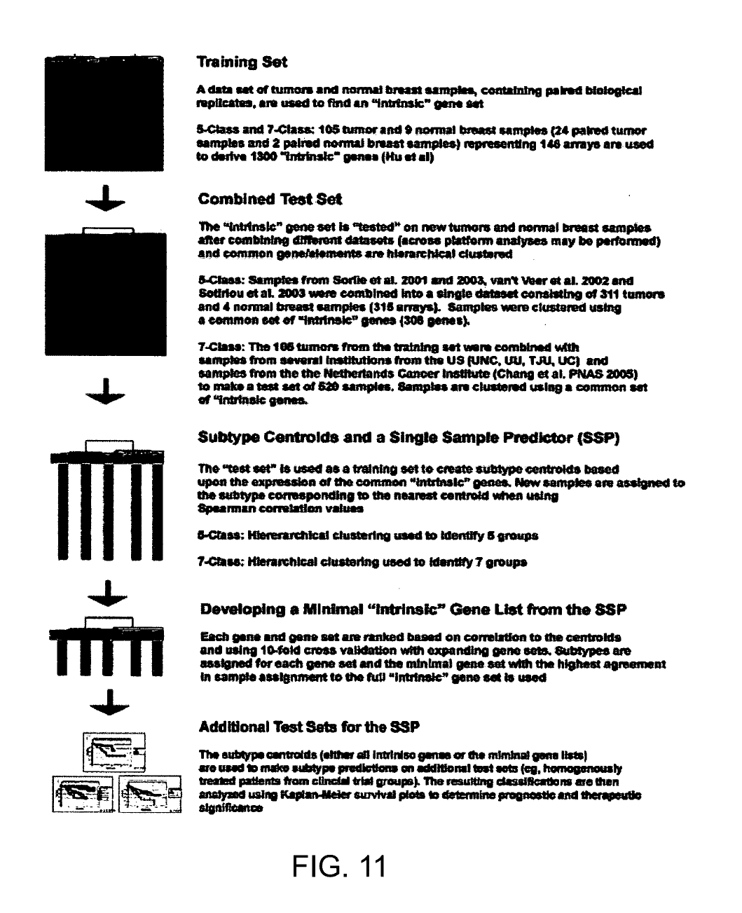

In the sixth, and final step, the smallest gene set which provides the highest class prediction accuracy when compared to the classifications made by the complete microarray-based intrinsic gene set is chosen. Subtypes are assigned for each gene set and the minimal gene set with the highest agreement in sample assignment to the full intrinsic gene set is chosen, hi one example, using a 1410 intrinsic gene set as disclosed in Example 2, 100 genes were identified (see Table 12 (7p 100), after the “Examples” section) that are important for identifying 7 different biological classes of breast cancer. Specific steps and sample sets used to develop the 7-class predictor as shown in FIG. 11. Also disclosed in Table 13 is an extended list of genes for classification resulting from the 7p analyses. This list is ranked in terms of significance for separating the different classes of intrinsic classifier genes. Another set of intrinsic genes that can be used for classification is found in Table 21, along with the primers that can be used to amplify those genes. It should be noted that the primers are optional and exemplary only, as any primer that can amplify a given gene can be used.

The minimal intrinsic gene set (identified using the methods described above, and found in Tables 12 and 13) has prognostic and predictive significance in breast cancer. The complete assay for making these biological “intrinsic” classifications includes 3 “housekeeper” genes (MRPL1 9, PUM1, and PSMC4) for normalizing the quantitative data. In addition, it has been shown that proliferation genes can also be used in combination with the housekeeper genes for providing a quantitative measurement of grade and for assessing prognosis in breast cancer.

Also disclosed herein is the Single Sample Predictor (SSP). The Single Sample Predictor/SSP is based upon the Nearest Centroid method presented in (Hastie et al. 2001). The subtype centroids (either all intrinsic genes or the minimal gene lists) can be used to make subtype predictions on additional test sets (e.g., homogenously treated subjects from clinical trial groups). The resulting classifications are then analyzed using Kaplan-Meier survival plots to determine prognostic and therapeutic significance. An example of SSP can be found in Example 2.

1. Intrinsic Genes and Cancer

An intrinsic gene is a gene that shows little variance within repeated samplings of the same tumor, but which shows high variance across tumors. Disclosed herein are genes that can be used as intrinsic genes with the methods disclosed herein. The intrinsic genes disclosed herein can be genes that have less than or equal to 0.00001, 0.0001, 0.001, 0.01, 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 1,000, 10,000, or 100,000% variation between two samples from the same tissue. It is also understood that these levels of variation can also be applied across 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more tissues, and the level of variation compared. It is also understood that variation can be determined as discussed in the examples using the algorithms as disclosed herein.

“Intrinsic gene set” is defined herein as comprising one or more intrinsic genes. “Minimal intrinsic gene set” is defined herein as being derived from an intrinsic gene set, and is considered the fewest number of intrinsic genes that can be used to classify a sample.

Disclosed herein is a set of 212 minimal intrinsic genes, as found in Table 21. These genes can be used alone, or in combination, as intrinsic genes for the purposes of classification, prognosis, and diagnosis of cancer, for example. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154. 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 of the genes can be used with the methods disclosed herein for analyzing samples.

Described herein is a method of diagnosing cancer, the method comprising comparing expression levels of a combination of genes from Table 21 to test nucleic acids corresponding to the same combination of genes, wherein specific expression patterns of the test nucleic acids indicates a cancerous state.

Also disclosed is a method of quantitating level of expression of a test nucleic acid comprising: a) comparing gene expression levels of a combination of genes from Table 21 to test nucleic acids corresponding to the same combination of genes; and b) quantitating level of expression of the test nucleic acid.

Also disclosed is a method of prognosing outcome in a subject diagnosed with cancer comprising: a) comparing expression levels of a combination of genes from Table 21 to test nucleic acids corresponding to the same combination of genes, b) identifying a subtype of cancer of the subject, and c) prognosing the outcome based on the subtype of cancer of the subject.

The intrinsic genes disclosed herein can be normalized to control housekeeper genes and used in a qRT-PCR diagnostic assay that uses relative copy number to assess risk or therapeutic response in cancer. For example, MRPL19 (SEQ ID NO:1), PSMC4 (SEQ ID NO:2), SF3A1 (SEQ IDNO:3), PUM1 (SEQ ID NO:4), ACTB (SEQ ID NO:5) and GAPD (SEQ ID NO:6). Other genes include GUSB, RPLPO, and TFRC, whose sequences can be found in Genbank. These are part of the 212 gene list. Other genes as disclosed herein can also be considered intrinsic genes.

The intrinsic genes can be used in any combination or singularly in any method described herein. It is also understood that any nucleic acid related to the expression control genes, such as the RNA, mRNA, exons, introns, or 5′ or 3′ upstream or downstream sequence, or DNA or gene can be used or identified in any of the methods or with any of the compositions disclosed herein.

2. Molecules for Detecting Genes, Gene Expression Products, Proteins Encoded by Genes

The disclosed methods involve using specific intrinsic genes or gene sets or expression control genes or gene sets such that they are detected in some way or their expression product is detected in some way. Typically the expression of a gene or its expression product will be detected by a primer or probe as disclosed herein. However, it is understood that they can also be detected by any means, such as in a microarray analysis or a specific monoclonal antibody or other visualization technique. Often, the expression of the genes of interest (control “housekeeper” genes or intrinsic classifier genes) can be detected after or during an amplification process, such as RT-PCR, including quantitative PCR.

3. Method of Diagnosing or Prognosing Cancer

Microarrays have shown that gene expression patterns can be used to molecularly classify various types of cancers into distinct and clinically significant groups. In order to translate these profiles into routine diagnostics, a microarray breast cancer classification system has been recapitulated using real-time quantitative (q)RT-PCR (Example 2). Statistical analyses were performed on multiple independent microarray datasets to select an “intrinsic” gene set that can classify breast tumors into four different subtypes designated as Luminal, Normal-like, HER2+/ER−, and Basal-like. Intrinsic genes, as described in Perou et al. (Nature (2000) 406:747-752), are statistically selected to have low variation in expression between biological sample replicates from the same individual and high variation in expression across samples from different individuals. Thus, intrinsic genes are the classifier genes for breast cancer classification and each classifier gene can be normalized to the housekeeper (or control) genes in order to make the classification. A minimal gene set from the microarray “intrinsic” list, and additional genes important for outcome (e.g., proliferation genes), were used to develop a real-time qRT-PCR assay comprised of 53 classifiers and 3 housekeepers. The expression data and classifications from microarray and real-time qRT-PCR were respectively compared using 123 unique breast samples (117 invasive carcinomas, 1 fibroadenoma and 5 normal tissues) and 3 cells lines. The overall correlation for the 50 genes in common between microarray and qRT-PCR was 0.76. There was 91% (114/126) concordance in the hierarchical clustering classification of the real-time qRT-PCR minimal “intrinsic” gene set (37 genes) and the larger (550 genes) microarray intrinsic gene set from which the PCR list was derived. As expected, the Luminal tumors (ER+) had a significantly better outcome than the HER2+/ER−(p=0.043) and Basal-like tumors (p=0.001). High expression of the proliferation genes GTBP4 (p=0.011), HSP A14 (p=0.023), and STK6 (p=0.027) were significant predictors of relapse free survival (RFS) independent of grade and stage. It has been shown that genomic microarray data can be translated into a qRT-PCR diagnostic assay that improves the standard of care in breast cancer.

The overlap in the minimized gene set discussed above and in Example 2 versus those in Example 3 is 14 out of 40. There are 108 genes in common between the larger intrinsic gene sets, which included 427 in Perreard et al versus 1300 used in Example 3. Example 2 illustrates how intrinsic gene sets can be minimized from microarray data and used on fresh tissue in a qRT-PCR assay to recapitulate the microarray classifications. It also shows the importance of the ‘proliferation’ genes in risk stratifying Luminal (ER+) breast tumors. Example 3 discusses a version of the intrinsic gene set from Hu et al and shows again how it can be minimized to provide intrinsic classifications on both fresh and FFPE tissue and using microarray or qRT-PCR data. Validated primer sequences from FFPE tissues for 212 genes important for breast cancer diagnostics are presented in Table 21.

A major challenge in the clinical care of cancer has been providing an accurate diagnosis for appropriate management of breast cancer. For over 50 years, medicine has relied on morphological features (histopathology) and anatomic staging (Tumor size/Node involvement/Metastasis) for classification of tumors (Greenough, R. B. J Cancer Res 9:452-463; Bloom et al. (1957) British Journal of Cancer 9:359-377). The TNM staging system provides information about the extent of disease and has been the “gold standard” for prognosis (Henson, et al. (1991) Cancer 68:2142-2149; Fitzgibbons, et al (2000) Arch Pathol Lab Med 124:966-978).

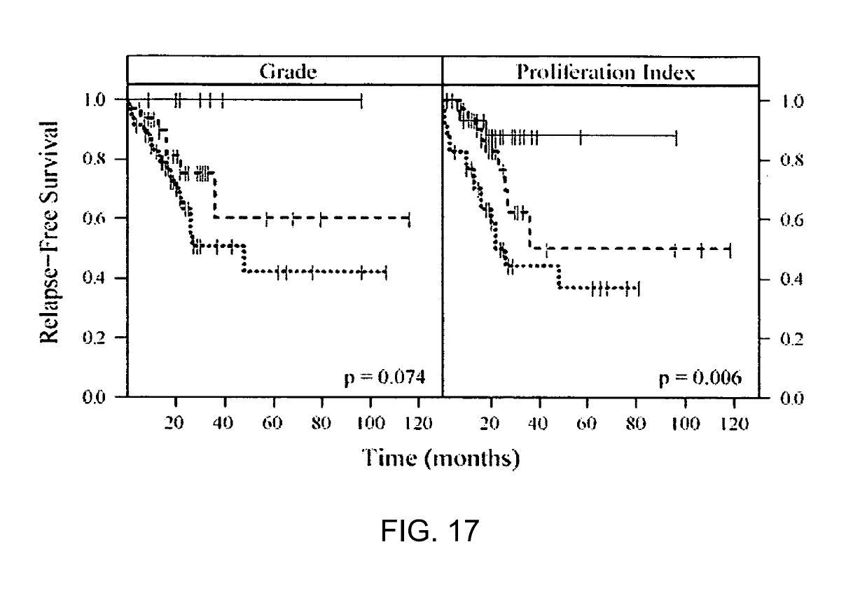

In addition to TNM, the grade of the tumor is also prognostic for relapse free survival (RFS) and overall survival (OS) (Elston et al. (1991) Histopathology 19:403-410). Tumor grade is determined from histological assessment of tubule formation, nuclear pleomorphism, and mitotic count. Due to the subjective nature of grading and difficulties standardizing methods, there has been less than optimal agreement between pathologists (Dalton et al. (1994) Cancer 73:2765-2770). Applying the Nottingham combined histological grade has made scoring more quantitative and improved agreement between observers (Frierson (1995) Am J Clin Pathol 103:195-198), however, more objective methods are still needed before grade is integrated into the TNM classification (Singletary (2003) Surg Clin North Am 83:803-819). For instance, most studies show significance in outcome between Grade 1 (low/least aggressive) and Grade 3 (high/most aggressive), but Grade 2 (intermediate) tumors show variability in outcome and are commonly not classified the same across institutions (Kollias et al. (1999) Eur J Cancer 35:908-912; Robbins et al. (1995) Hum Pathol 26:873-879; Genestie et al. (1998) Anticancer Res 18:571-576.). Alternatively, proliferation assays, such as S-phase fraction and mitotic index, have shown to be independent prognostic indicators and could be used in conjunction with, or instead of grade (Michels et al. (2004) Cancer 100:455-464; CaIy et al. (2004) Anticancer Res 24:3283-3288). It has been shown that proliferation genes can be used in a qRT-PCR assay and the genes can be averaged to produce a proliferation meta-gene that correlates with grade but is more prognostic (FIG. 17).

Women with the same stage of breast cancer can have widely different clinical outcomes due to differences in tumor biology (van't Veer et al. (2002) Nature 415:530-536; van van de Vijver et al. (2002) N Engl J Med 347:1999-2009. The use of gene expression markers in breast pathology can provide addition clinical information that complements the TNM system for prognosis and is important for making therapeutic decisions (van't Veer et al. (2002) Nature 415:530-536; van de Vijver et al. (2002) N Engl J Med 347:1999-2009; Paik et al. (2004) N Engl J Med 351:2817-2826; Sørlie et al. (2001) Proc Natl Acad Sci USA 98:10869-10874; Sorlie et al. (2003) Proc Natl Acad Sci USA 100:8418-8423). Undoubtedly, one of the greatest advancements in breast cancer medicine has been the identification and routine testing for the expression of the hormone receptors, namely the Estrogen Receptor (ER) and the Progesterone Receptor (PgR), which allows the clinician to offer endocrine blockade therapy that can significantly prolong survival in women with tumors expressing these proteins (Buzdar et al. (2003) J Clin Oncol 21:1007-1014; Fisher et al (1989) N Engl J Med 320:479-484).

Although ER expression is a predictive marker, it also serves as a surrogate marker for describing a tumor biology that is characteristically less aggressive (e.g. lower grade) than ER-negative tumors (Fisher et al. (1981) Breast Cancer Res Treat 1:37-41). Microarrays have elucidated the richness and diversity in the biology of breast cancer and have identified many genes that associate with ER-positive and ER-negative tumors (Perou et al. (2000) Nature 406:747-752; West et al. (2001) Proc Natl Acad Sci USA 98:11462-11467; Gruvberger et al. (2001) Cancer Res 61:5979-5984). When microarray data from invasive breast carcinomas are analyzed by hierarchical clustering, samples are separated primarily based on ER status (Sotiriou et al. (2003) Proc Natl Acad Sci USA 100:10393-10398).

Breast tumors of the “Luminal” subtype are ER positive and have a similar keratin expression profile as the epithelial cells lining the lumen of the breast ducts (Taylor-Papadimitriou et al. (1989) J Cell Sci 94:403-413; Perou et al. (2000) New Technologies for life sciences: A Trends Guide:67-76). Conversely, ER-negative tumors can be broken into two main subtypes, namely those that overexpress (and are DNA amplified for) HER2 and GRB7 (HER2+/ER−), and “Basal-like” tumors that have an expression profile similar to basal epithelium and express Keratin 5, 6B and 17. Both these tumor subtypes are aggressive and typically more deadly than Luminal tumors; however, there are subtypes of Luminal tumors that lead to poor outcome despite being ER-positive. For instance, Sorlie et al. identified a Luminal B subtype with similar outcomes to the HER2+/ER− and Basal-like subtypes, and Sotiriou et al. showed that there are 3 different types of Luminal tumors with different outcomes. The Luminal tumors with poor outcomes consistently share the histopathological feature of being higher grade and the molecular feature of highly expressing proliferation genes.

The so called “proliferation genes” show periodicity in expression through the cell cycle and have a variety of functions necessary for cell growth, DNA replication, and mitosis (Whitfield et al. (2002) Mol Biol Cell 13:1977-2000; Ishida et al. Mol Cell Biol 21:4684-4699). Despite their diverse functions, proliferation genes have similar gene expression profiles when analyzed by hierarchical clustering. As might be expected, proliferation genes correlate with grade, the mitotic index (Perou et al. (1999) Proc Natl Acad Sci USA 96:9212-9217), and outcome (Sørlie et al. (2001) Proc Natl Acad Sci USA 98:10869-10874). Proliferation genes are often selected when supervised analysis is used to find genes that correlate with patient outcome. For example, the SAM264 “survival” list presented in Sorlie et al., the 231 “prognosis classifier” list in van't Veer et al., and the “485 prognostic gene” list in Sotiriou et al., identified common proliferation genes (PCNA, TOP2A, CENPF). This suggests that all these studies are likely tracking a similar phenotype.

Gene expression profiling using DNA microarrays is a powerful tool to discover genes for molecular classifications of cancer but the platforms are labor intensive, expensive and currently not amenable to routine clinical diagnostics. Real-time qRT-PCR is well-suited for solid tumor diagnostics since it is rapid, homogenous (amplification and quantification in a single vessel), and can be performed from archived (FFPE tissue) samples. Example 3 shows that FFPE samples can perform as well as fresh samples. It has been shown that “intrinsic” breast cancer classifications from microarray can be recapitulated by qRT-PCR using a minimal “intrinsic” gene set. In addition, by supplementing the “intrinsic” gene set with proliferation genes, a more objective measurement of grade has been developed. The assay disclosed herein adds prognostic information to the standard of care for breast cancer.

Microarray used in conjunction with RT-PCR provides a powerful system for discovering and translating genomic markers into the clinical laboratory for molecular diagnostics. Although these platforms are fundamentally very different, the quantitative data across the methods have a high correlation. In fact, the data across the methods is no more disparate then across different microarray platforms. By hierarchical clustering, it has been shown that a biological classification of breast cancer derived from microarray data can be recapitulated using real-time qRT-PCR. Biological classification by real-time qRT-PCR makes the important clinical distinction between ER positive and ER negative tumors and identifies additional subtypes that have prognostic (i.e., correlate to outcome) and predictive value (i.e., correlate to treatment response).

The benefit of using real-time qRT-PCR for cancer diagnostics is that new informative markers can be readily validated and implemented, making tests expandable and/or tailored to the individual. For instance, it has been shown that including proliferation genes serves a similar purpose to grade but is more prognostic. Since grade has been shown to be universal as a prognostic factor in cancer, it is likely that the same markers correlate to grade and are important for survival in other tumor types. Real-time qRT-PCR is attractive for clinical use because it is fast, reproducible, tissue sparing, and able to be automated. Although genomic profiling should currently be used for ancillary testing, the fact that normal tissues can be distinguished from tumor tissue shows that these molecular assays may eventually be used for cancer diagnostics without histological corroboration.

Disclosed is a method of classifying cancer in a subject, comprising: a) identifying intrinsic genes of the subject to be used to classify the cancer; b) obtaining a sample from the subject; c) amplifying and detecting levels of intrinsic genes in the subject; and d) classifying cancer based upon results of step c. The sample can be fresh, or can be an FFPE sample.

Also disclosed is a method of diagnosing cancer in a subject the method comprising: a) amplifying and detecting intrinsic genes; and b) diagnosing cancer based on expression levels of the gene within the subject. The methods disclosed herein can be used with any of the types of cancer listed herein. The cancer can be breast cancer, for example. The breast cancer can be classified into one of four or more groups: luminal, normal-like, HER2+/ER− and basal-like, for example. Again, the sample can be fresh, or can be an FFPE sample.

Disclosed are methods of analyzing nucleic acid expression levels in a sample, the methods comprising comparing expression levels of an intrinsic gene set to a test nucleic acid, wherein specific expression patterns of the test gene relative to the intrinsic gene set indicates a diagnoses, poor prognosis, likelihood of obtaining, predisposition to obtaining, or presence of a cancer. Also disclosed are methods wherein the step of comparing comprises identifying the expression levels of an intrinsic gene set and a test nucleic acid by interaction with a primer or probe.

Disclosed are methods where a specific expression pattern of a test nucleic acid relative to an intrinsic gene set indicates the presence of a cancer, a poor (or good) prognosis for a patient having a cancer, a predisposition of getting a cancer, or a diagnoses of cancer or a cancerous state.

It is understood that any method of assaying any gene discussed herein can be performed. For example methods of assaying gene copy number or mRNA expression copy number can be performed. For example, RT-PCR, PCR, quantitative PCR, and any other forms of nucleic acid amplification can be performed. Furthermore, methods of hybridization, such as blotting, such as Northern or Southern techniques, such as chip and microarray techniques and any other techniques involving hybridizing of nucleic acids.

4. A Non-Limiting List of Cancers which can be Assayed with Disclosed Compositions and Methods

The disclosed compositions can be used to diagnose or prognose any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to diagnose or prognose is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Compounds disclosed herein may also be used for the diagnosis or prognosis of precancer conditions such as cervical and anal dysplasias, other dysplasias, severe dysplasias, hyperplasias, atypical hyperplasias, and neoplasias.

5. Methods of Identifying a Minimal Intrinsic Gene Set

Disclosed are methods of identifying minimal intrinsic genes. These methods are described in detail above, and generally comprise the following: deriving a minimal intrinsic gene set for making biological classifications of cancer comprising: a) collecting data from multiple samples from the same or different individuals to identify potential intrinsic classifier genes (microarray data can be used in this step, for example); b) weighting intrinsic classifier genes of multiple individuals identified using the method of step a relative to each other and forming classification clusters (weighting can be done, for example, by forming hierarchical clusters); c) estimating the number of clusters formed in step b) and assigning individual samples to clusters; d) identifying genes that optimally distinguish the samples in the assigned groups of step c); e) performing iterative cross-validation with a nearest centroid classifier and overlapping gene sets of various sizes using the genes identified in step d); and f) choosing a gene set which provides the highest class prediction accuracy when compared to the classifications made in step b).

Also disclosed is a method of assigning a sample to an intrinsic subtype, ′comprising a) creating an intrinsic subtype average profile (centroid) for each subtype; b) individually comparing a new sample to each centroid; and c) assigning the new sample to the centroid that is most similar to the new sample. This is known as the Single Sample Predictor (SSP) method, and is described in further detail in Example 2.

Also disclosed are computerized implementing systems, as well as storage and retrieval systems, of biological information, comprising: a data entry means; a display means; a programmable central processing unit; and a data storage means having expression data for a gene electronically stored; wherein the stored sequences are used as input data for determining which sequence is the best intrinsic gene set for a specific tissue type.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular expression control gene is disclosed and discussed and a number of modifications that can be made to a number of molecules including the expression control gene are discussed, specifically contemplated is each and every combination and permutation of expression control gene and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the subgroup of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5′C to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Nucleic Acids

There are a variety of molecules disclosed herein that are nucleic acid based, including for example the nucleic acids that encode, for example, the intrinsic genes disclosed herein (Table 12), as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

b) Primers and Probes

It is understood that primers and probes can be produced for the actual gene (DNA) or expression product (mRNA) or intermediate expression products which are not fully processed into mRNA. Discussion of a particular gene is also a disclosure of the DNA, mRNA, and intermediate RNA products associated with that particular gene.

Disclosed are compositions including primers and probes, which are capable of interacting with the intrinsic genes disclosed herein, as well as the any other genes or nucleic acids discussed herein, hi certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed genes or regions of the disclosed genes or they hybridize with the complement of the disclosed genes or complement of a region of the disclosed genes.

The size of the primers or probes for interaction with the disclosed genes in certain embodiments can be any size that supports the desired enzymatic manipulation of the primer, such as DNA amplification or the simple hybridization of the probe or primer. A typical disclosed primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 61, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the disclosed primers or probes can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the disclosed genes in certain embodiments can be used to produce an amplified DNA product that contains the desired region of the disclosed genes. In general, typically the size of the product will be such that the size can be accurately determined to within 10, 5, 4, 3, or 2 or 1 nucleotides.

In certain embodiments this product is at least 20, 21, 22, 23, 24, 25, 27, 28 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 61, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 61, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In certain embodiments the primers and probes are designed such that they are targeting as specific region in one of the genes disclosed herein. It is understood that primers and probes having an interaction with any region of any gene disclosed herein are contemplated: In other words, primers and probes of any size disclosed herein can be used to target any region specifically defined by the genes disclosed herein. Thus, primers and probes of any size can begin hybridizing with nucleotide 1, 2, 3, 4, 5, 6, 7, 8, 9, 10, or any specific nucleotide of the genes or gene expression products disclosed herein. Furthermore, it is understood that the primers and probes can be of a contiguous nature meaning that they have continuous base pairing with the target nucleic acid for which they are complementary. However, also disclosed are primers and probes which are not contiguous with their target complementary sequence. Disclosed are primers and probes which have at least 1, 2, 3, 4, 5, 6, 7, 8, 9, 10, 15, 20, 25, 30, 35, 40, 50, 75, 100, 150, 200, 500, or more bases which are not contiguous across the length of the primer or probe. Also disclosed are primers and probes which have less than or equal to 1, 2, 3, 4, 5, 6, 7, 8, 9, 10, 15, 20, 25, 30, 35, 40, 50, 75, 100, 150, 200, 500, or more bases which are not contiguous across the length of the primer or probe.

In certain embodiments the primers or probes are designed such that they are able to hybridize specifically with a target nucleic acid. Specific hybridization refers to the ability to bind a particular nucleic acid or set of nucleic acids preferentially over other nucleic acids. The level of specific hybridization of a particular probe or primer with a target nucleic acid can be affected by salt conditions, buffer conditions, temperature, length of time of hybridization, wash conditions, and visualization conditions. By increasing the specificity of hybridization means decreasing the number of nucleic acids that a given primer or probe hybridizes to typically under a given set of conditions. For example, at 20 degrees Celsius under a given set of conditions a given probe may hybridize with 10 nucleic acids in a sample. However, at 40 degrees Celsius with all other conditions being equal, the same probe may only hybridize with 2 nucleic acids in the same sample. This would be considered an increase in specificity of hybridization. A decrease in specificity of hybridization means an increase in the number of nucleic acids that a given primer or probe hybridizes to typically under a given set of conditions. For example, at 700 mM NaCl under a given set of conditions a particular probe or primer may hybridize with 2 nucleic acids in a sample, however when the salt concentration is increased to 1 Molar NaCl the primer or probe may hybridize with 6 nucleic acids in the same sample.

The salt can be any salt such as those made from the alkali metals: Lithium, Sodium, Potassium, Rubidium, Cesium, or Francium or the alkaline earth metals: Beryllium, Magnesium, Calcium, Strontium, Barium, or Radiumsodium, or the transition metals: Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Hafnium, Tantalum, Tungsten, Rhenium, Osmium, Iridium, Platinum, Gold, Mercury, Rutherfordium, Dubniuni, Seaborgium, Bohrium, Hassium, Meitnerium, Ununnilium, Unununium or Unuribium at any molar strength to promoter the desired condition, such as 1, 0.7, 0.5, 0.3, 0.2, 0.1, 0.05, or 0.02 molar salt, ha general increasing salt concentration decreases the specificity of a given probe or primer for a given target nucleic acid and decreasing the salt concentration increases the specificity of a given probe or primer for a given target nucleic acid.

The buffer conditions can be any buffer such as TRIS at any pH, such as 5.0, 5.5, 6.0, 6.1, 6.2, 6.3, 6.4, 6.5, 6.6, 6.1, 6.8, 6.9, 7.0, 7.1, 7.2, 7.3, 7.4, 7.5, 7.6, 7.7, 7.8, 7.9, 8.0, 8.5, or 9.0. In general pHs above or below 7.0 increase the specificity of hybridization.

The temperature of hybridization can be any temperature. For example, the temperature of hybridization can occur at 20°, 21°, 22°, 23°, 24°, 25°, 26°, 27°, 28°, 29°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 81°, 82°, 83°, 84°, 85°, 86°, 87°, 88°, 89°, 90°, 91°, 92°, 93°, 94°, 95°, 96°, 97°, 98°, or 99° Celsius.

The length of time of hybridization can be for any time. For example, the length of time can be for 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 150, 180, 210, 240, 270, 300, 360, minutes or 7, 8, 9, 10, 11, 12, 13, 14, 15, 16, 17, 18, 19, 20, 21, 22, 23, 24, 30, 36, 48 or more hours.

It is understood that any wash conditions can be used including no wash step. Generally the wash conditions occur by a change in one or more of the other conditions designed to require more specific binding, by for example increasing temperature or decreasing the salt or changing the length of time of hybridization.

It is understood that there are a variety of visualization conditions which have different levels of detection capabilities. Li general any type of visualization or detection system can be used. For example, radiolabeling or fluorescence labeling can be used and in general fluorescence labeling would be more sensitive, meaning a fewer number of absolute molecules would have to be present to be detected.

c) Sequences

There are a variety of sequences related to the intrinsic genes as well as the others disclosed herein and others are herein incorporated by reference in their entireties as well as for individual subsequences contained therein. A specific intrinsic gene set can be found in Table 12.

4. Kits

Disclosed are kits comprising nucleic acids which can be used in the methods disclosed herein and, for example, buffers, salts, and other components to be used in the methods disclosed herein. Disclosed are kits for identifying minimal intrinsic gene sets comprising nucleic acids, such as in a microarray. Also disclosed are specific minimal intrinsic genes used for classifying cancer, such as those found in Table 21. As described above, these intrinsic genes can be used in any combination or permutation, and any combination of permutation of these genes can be used in a kit. Also disclosed are kits comprising instructions.

5. Chips and Micro Arrays

Disclosed are chips where at least one address is the sequences or part of the sequences set forth in any of the nucleic acid sequences disclosed herein.

Also disclosed are chips where at least one address is a variant of the sequences or part of the sequences set forth in any of the nucleic acid sequences disclosed herein.

6. Computer Readable Mediums

Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

### D. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABI Model 380B). Synthetic methods useful for making oligonucleotides are also described by Dcuta et al., Ann. Rev. Biochem. 53:323-356 (1984), (phosphotriester and phosphite-triester methods), and Narang et al., Methods EnzymoL, 65:610-620 (1980), jfiosp otf es er metKo). Protein nucleic acid molecules can be made using known methods such as those described by Nielsen et ah, Bioconjug. Chem. 5:3-7 (1994).

### E. METHODS OF USING THE COMPOSITIONS

### 1. Methods of Using the Compositions as Research Tools

The disclosed compositions can be used in a variety of ways as research tools. The compositions can be used for example as targets in combinatorial chemistry protocols or other screening protocols to isolate molecules that possess desired functional properties related to the disclosed genes.

The disclosed compositions can also be used diagnostic tools related to diseases, such as cancers, such as those listed herein.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any method for determining allelic analysis of for example, the genes disclosed herein. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Biological Classification of Breast Cancer by Real-Time Quantitative RT-PCR: Comparisons to Microarray and Histopathology

a) Methods

Patient selection. An ethnically diverse cohort of patients were studied using samples collected from various locations throughout the United States. Tissues analyzed included 117 invasive breast cancers, 1 fibroadenoma, 5 “normal” samples (from reduction mammoplasty), and 3 cells lines. Patients were heterogeneously treated in accordance with the standard of care dictated by their disease stage, ER and HER2 status. Patients were censored for recurrence and/or death for up to 118 months (median 21.5 months). Clinical data presented in supplementary Table 7.

Sample preparation and first strand synthesis for qRT-PCR. Nucleic acids were extracted from fresh frozen tissue using RNeasy Midi Kit (Qiagen Inc., Valencia, Calif.). The quality of RNA was assessed using the Agilent 2100 Bioanalyzer with the RNA 6000 Nano LabChip Kit (Agilent Technologies, Palo Alto, Calif.). All samples used had discernable 18S and 28S ribosomal peaks. First strand cDNA was synthesized from approximately 1.5 mg total RNA using 500 ng Oligo(dT)12-18 and Superscript III reverse transcriptase (1st Strand Kit, Invitrogen, Carlsbad, Calif.). The reaction was held at 42° C. for 50 min followed by a 15-min step at 70° C. The cDNA was washed on a QIAquick PCR purification column and stored at −80° C. in TE′ (25 mM Tris, 1 mM EDTA) at a concentration of 5 ng/ul (concentration estimated from the starting RNA concentration used in the reverse transcription).

Primer design. Genbank sequences were downloaded from Evidence viewer (NCBI website) into the Lightcycler Probe Design Software (Roche Applied Science, Indianapolis, Ind.). All primer sets were designed to have a Tm>>60′C, GC content>>50% and to generate a PCR amplicon <200 bps. Finally, BLAT and BLAST searches were performed on primer pair sequences using the UCSC Genome Bioinformatics (http://genome.ucsc.edu/) and NCBI (http://www.ncbi.nhn.nih.gov/BLAST/) to check for uniqueness. Primer sets and identifiers are provided in supplementary Table 8.

Real-time PCR. For PCR, each 20 μL reaction included IX PCR buffer with 3 mM MgCl2 (Idaho Technology Inc., Salt Lake City, Utah), 0.2 mM each of dATP, dCTP, and dGTP, 0.1 mM dTTP, 0.3 mM dUTP (Roche, Indianapolis, Ind.), 10 ng cDNA and IU Platinum Taq (Invitrogen, Carlsbad, Calif.). The dsDNA dye SYBR Green I (Molecular Probes, Eugene, Oreg.) was used for all quantification ( 1/50000 final). PCR amplifications were performed on the Lightcycler (Roche, Indianapolis, Ind.) using an initial denaturation step (94° C., 90 sec) followed by 5 cycles: denaturation (94° C., 3 sec), annealing (58° C., 5 sec with 20° C./s transition), and extension (72° C., 6 sec with 2° C./sec transition). Fluorescence (530 nm) from the dsDNA dye SYBR Green I was acquired each cycle after the extension step. Specificity of PCR was determined by post-amplification melting curve analysis. Reactions were automatically cooled to 60° C. at a rate of 3° C./s and slowly heated at 0.1° C./s to 95° C. while continuously monitoring fluorescence.

Relative quantification by RT-PCR. Quantification was performed using the LightCycler 4.0 software. The crossing threshold (Ct) for each reaction was determined using the 2nd derivative maximum method (Wittwer et al. (2004) Washington, D.C.: ASM Press; Rasmussen (2001) Heidelberg: Springer Verlag. 21-34). Relative copy number was calculated using an external calibration curve to correct for PCR efficiency and a within run calibrator to correct for the variability between run. The calibrator is made from 4 equal parts of RNA from 3 cell lines (MCF7, SKBR3, ME16C) and Universal Human Reference RNA (Stratagene, La Jolla, Calif., Cat #740000). Differences in cDNA input were corrected by dividing target copy number by the arithmetic mean of the copy number for 3 housekeeper genes (MRPLl9, PSMC4, and PUM1) (Szabo et al. (2004) Genome Biol 5:R59). The normalized relative gene copy number was Iog2 transformed and analyzed by hierarchical clustering using Cluster (Eisen et al. (1998) Proc Natl Acad Sci USA 95:14863-14868). The clustering was visualized using Treeview software (Eisen Lab, http:/rana.Ibl.gov/EisenSoftware.htm).

Microarray experiments. The same 126 samples used for qRT-PCR were analyzed by microarray (Agilent Human oligonucleotide). Total RNA was prepared and quality checked as described above. Labeling and hybridization of RNA for microarray was done using the Agilent low RNA input linear amplification kit (http://www.chem.agilent.com/Scripts/PDS.asp?lPage=10003), but with one-half the recommended reagent volumes and using a Qiagen PCR purification kit to clean up the cRNA. Each sample was assayed versus a common reference sample that was a mixture of Stratagene's Human Universal Reference total RNA (lOOug) enriched with equal amounts of RNA (0.3 μg each) from MCF7 and ME16C cell lines. Microarray hybridizations were carried out on Agilent Human oligonucleotide microarrays (1 A-v1, 1 A-v2 and custom designed 1 A-v1 based microarrays) using 2 μg each of Cy3-labeled “reference” and Cy5-labeled “experimental” sample. Hybridizations were done using the Agilent hybridization kit and a Robbins Scientific “22k chamber” hybridization oven. The arrays were incubated overnight and then washed once in 2×SSC and 0.0005% triton X-102 (10 min), twice in 0.1×SSC (5 min), and then immersed′ into Agilent Stabilization and Drying solution for 20 seconds. All microarrays were scanned using an Axon Scanner 4000A. The image files were analyzed with GenePix Pro 4.1 and loaded into the UNC Microarray Database at the University of North Carolina at Chapel Hill (https://genome.unc.edu/) where a lowess normalization procedure was performed to adjust the Cy3 and Cy5 channels (Yang et al. (2002) Nucleic Acids Res 30:e15). All primary microarray data associated with this study are available at the UNC Microarray Database and have been deposited into the GEO (http://www.ncbi.nlm.nih.gov/geo/) under the accession number of GSE1992, series GSM34424-GSM34568.

Selecting genes for real-time qRT-PCR. A new “intrinsic” gene set for classifying breast tumors was derived using 45 before and after therapy samples from the combined data sets presented in Sorlie et al. (see Table 9 for the list of 45 pairs). The two-color DNA microarray data sets were downloaded from the internet and the R/G ratio (experimental/reference) for each spot was normalized and Iog2 transformed. Missing values were imputed using the k-NN imputation algorithm described by Troyanskaya et al. (Troyanskaya et al. (2001) Bioinformatics 17:520-525). The “intrinsic” analysis identified 550 gene elements.

Next, a completely independent data set was utilized (van't Veer et al. 2002) to derive an optimized version of the 550 intrinsic gene list. To allow across data set analyses, gene annotation from each dataset was translated to UniGene Cluster IDs (UCID) using the SOURCE database (Diehn et al. (2003) Nucleic Acids Res 31:219-223). Following the algorithm outlined by Tibshirani and colleagues (Bair et al. (2004) PLoS Biol 2:E108; Bullinger et al. (2004) N Engl J Med 350:1605-1616), the 97 samples from the van't Veer et al. 2002 study were hierarchical clustered using a common set of 350 genes, and assigned an “intrinsic subtype of either Luminal, HER2+/ER−, Basal-like, or Normal-like to each sample. A feature/gene selection was then performed to identify genes that optimally distinguished these 4 classes using a version of the gene selection method first described by Dudoit et al. (Genome Biol 3:RESEARCH0036), where the best class distinguishers are identified according to the ratio of between-group to within-group sums of squares (a type of ANOVA). In addition to statistically selecting “intrinsic” classifiers proliferation genes (e.g., TOP2A, KI-67, PCNA) were also chosen, and other important prognostic markers (e.g., PgR) that have potential for diagnostics. In total, 53 differentially expressed biomarkers were used in the real-time qRT-PCR assay (Table 8).

Combining microarray and qRT-PCR datasets. Distance Weighted Discrimination (DWD) was used to identify and correct systematic biases across the microarray and qRT-PCR datasets (Benito et al. (2004) Bioinformatics 20:105-114). Prior to DWD, each dataset was normalized by setting the mean to zero and the variance to one. Normalization was done within each microarray experiment and for genes profiled across many experimental runs for real-time qRT-PCR. After DWD, genes in common between the datasets were clustered using Spearman correlation and average linkage association.

Receiver operator curves. In order to determine agreement between protein expression (immunohistochemistry) and gene expression (qRT-PCR), a cut-off for relative gene copy number was selected by minimizing the sum of the observed false positive and false negative errors. That is, minimizing the estimated overall error rate under equal priors for the presence/absence of the protein. The sensitivity and specificity of the resulting classification rule were estimated via bootstrap adjustment for optimism (Efron et al. (1998) CRC Press LLC. p 247 pp).

Survival analyses. Survival curves were estimated by the Kaplan-Meier method and compared via a log-rank or stratified log-rank test as appropriate. Standard clinical pathological parameters of age (in years), node status (positive vs. negative), tumor size (cm, as a continuous variable), grade (1-3, as a continuous covariate), and ER status (positive vs. negative) were tested for differences in RFS and OS using Cox proportional hazards regression model. Pairwise log-rank tests were used to test for equality of the hazard functions among the intrinsic classes. Only the classes Luminal, HER2+/ER−, and Basal-like classes were included in the analyses because it was believed the Normal Breast-like subtype is not a pure tumor class and may result from normal breast contamination. Cox regression was used to determine predictors of survival from continuous expression data. All statistical analyses were performed using the R statistical software package (R Foundation for Statistical Computing).

b) Results

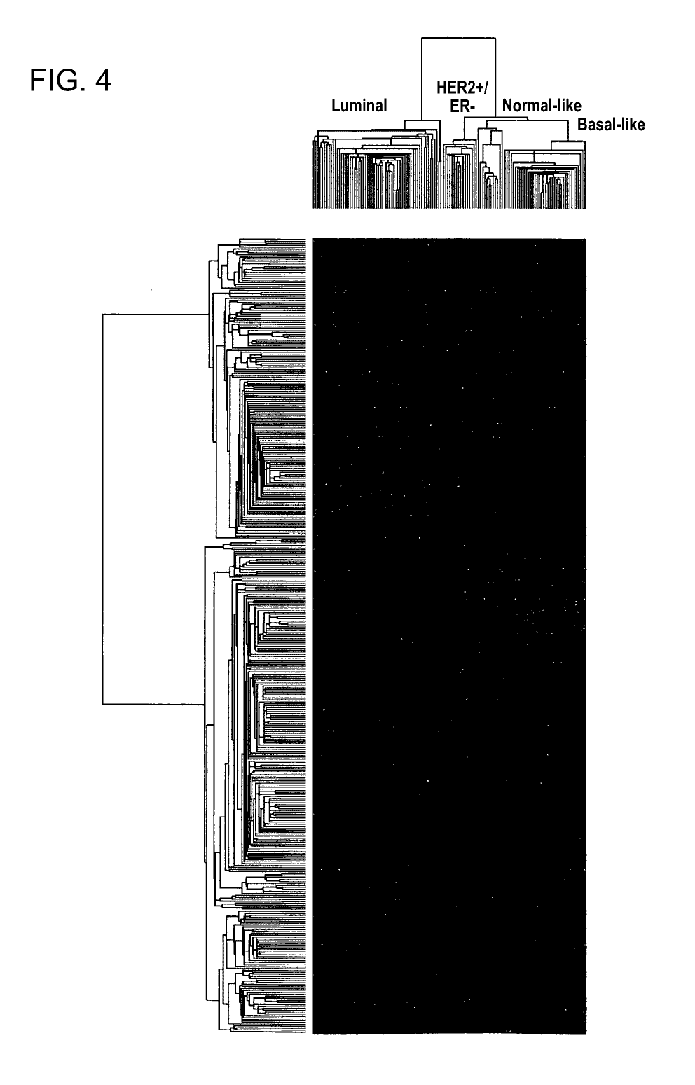

Recapitulating microarray breast cancer classifications by qRT-PCR. 126 different breast tissue samples (117 invasive, 5 normal, 1 fibroadenoma, and 3 cell lines) were expression profiled using a real-time qRT-PCR assay comprised of 53 biological classifiers and 3 control/housekeepers genes. Genes were statistically selected to optimally identify the 4 main breast tumor intrinsic subtypes, and to create an objective gene expression predictor for cell proliferation and outcome (Ross et al. (2000) Nat Genet 24:227-235). There were 402 genes in common between this microarray dataset and the 550 “intrinsic” genes selected from the Sorlie et al. 2003 study. Two-way hierarchical clustering of the 402 genes in the microarray gave the same tumor subtypes as the minimal 37 “intrinsic” genes assayed by qRT-PCR (FIG. 4). The samples were grouped into Luminal, HER2+/ER−, Normal-like, and Basal-like subtypes. Out of 123 breast samples compared across the platforms, 114 (93%) were classified the same. The minimal “intrinsic” gene set identified expression signatures within the 3 different cell lines that were characteristic of each tumor subtype: Luminal (MCF7), HER2+/ER− (SKBR3), and Basal-like (MEl 6C). The genes EGFR and PgR, which were added for their predictive and prognostic value in breast cancer Nielsen et al. (2004) Clin Cancer Res 10:5367-5374; Makretsov et al. (2004) Clin Cancer Res 10:6143-6151), had opposite expression and were found to associate with either ER-positive tumors (high expression of PgR) or ER-negative tumors (high expression of EGFR) (FIG. 4C).

Proliferation and grade. Expression of the 14 “proliferation” genes (FIG. 4D) assayed by qRT-PCR showed that Luminal tumors have relatively low replication activity compared to HER2+/ER− and Basal-like tumors. As expected, the Normal-like samples showed the lowest expression of the “proliferation” genes. When correlating (Spearman correlation) the gene expression of all 53 genes with grade, it was found that the top 3 proliferation genes with a positive correlation (i.e., high expression correlates with high grade) were the proliferation genes CENPF (p=2.00E-07), BUB1 (p=6.84E-07), and STK6 (p=2.67E-06) (see supplementary Table 10). Interestingly, all the proliferation genes, except PCNA, were at the top of the list for having a positive correlation to grade. Conversely, the top markers with significant negative correlations with grade (i.e., low expression correlates with high grade) were GATA3 (p=3.53E-07), XBP1 (p=9.64E-06), and ESR1 (p=4.53E-05).

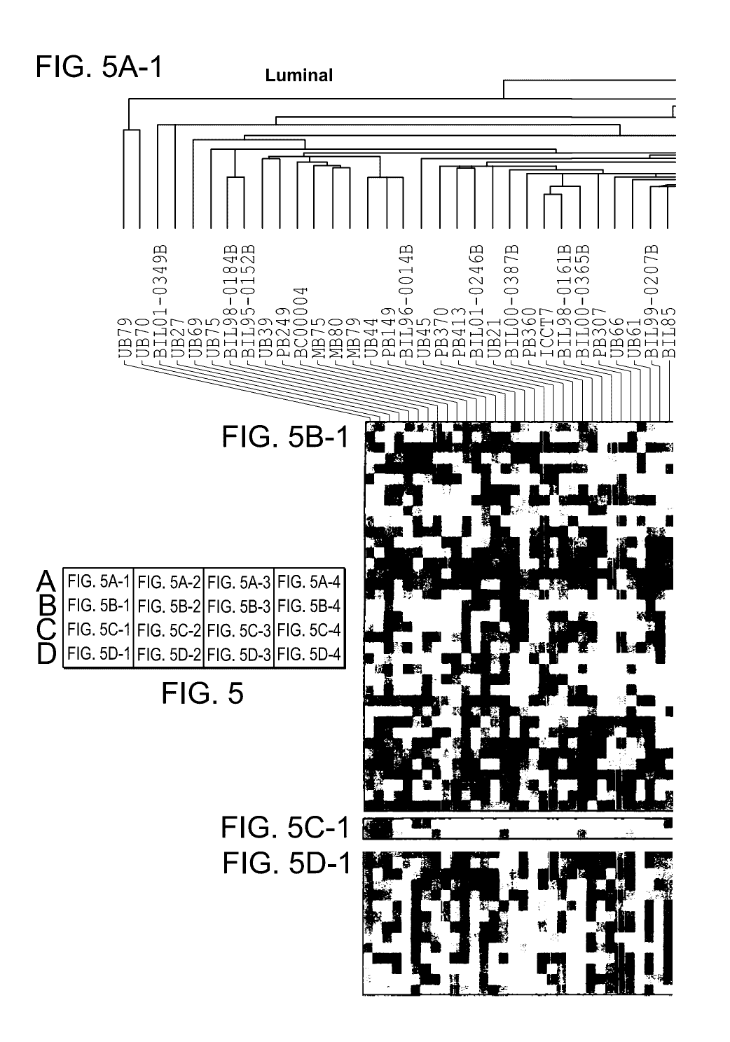

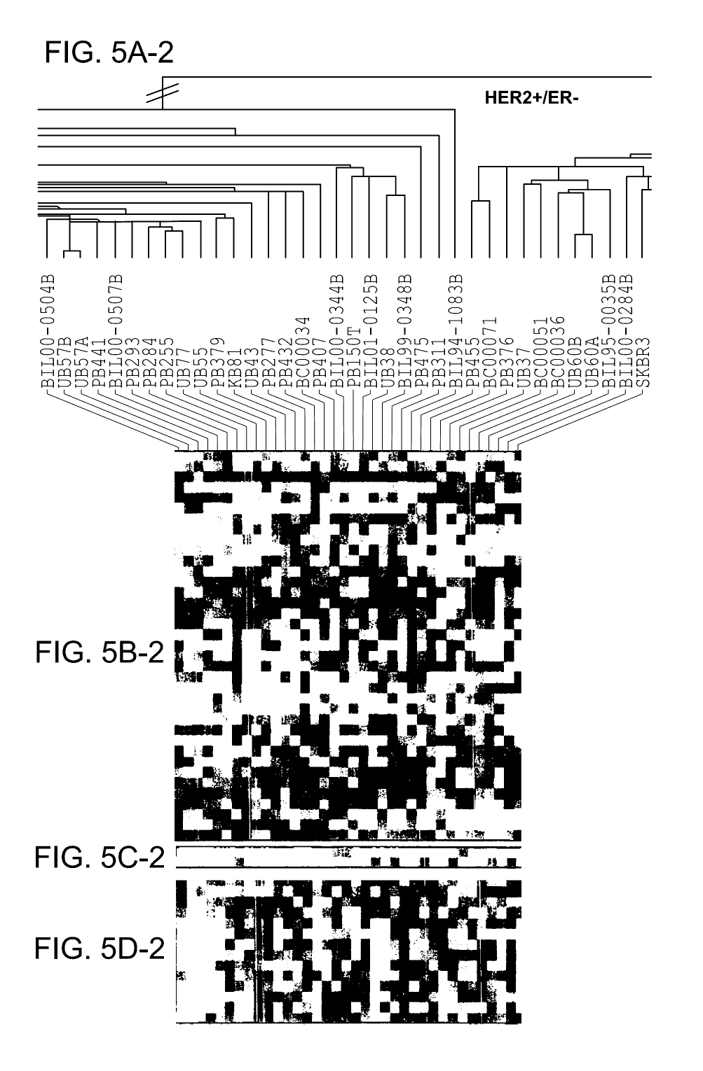

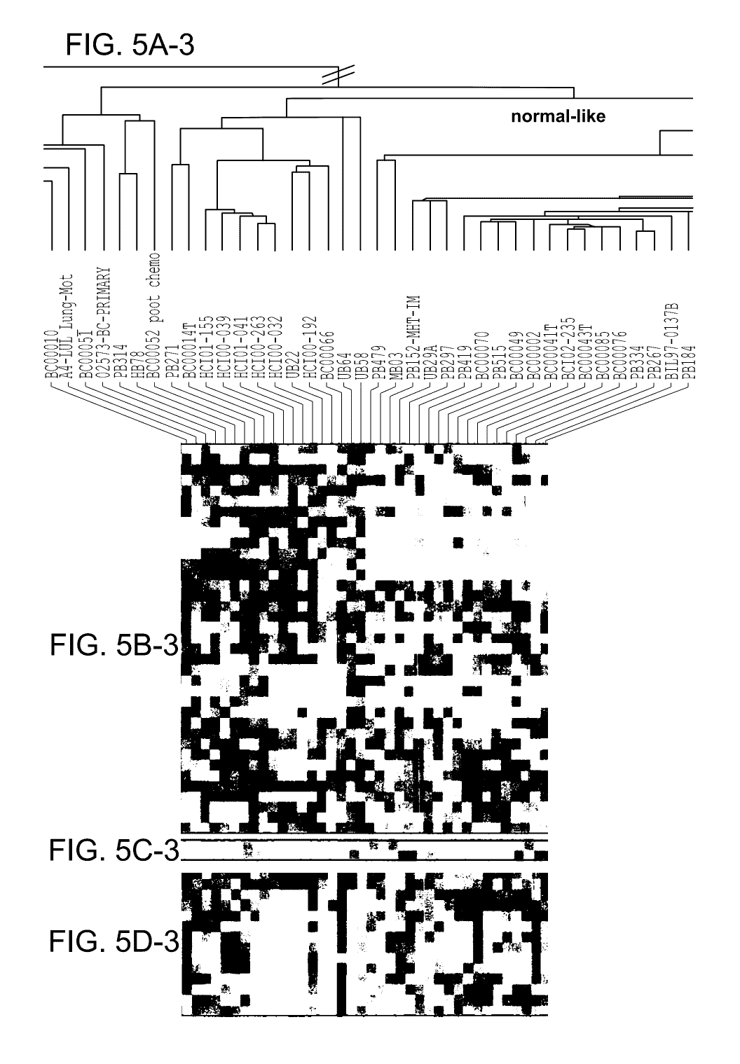

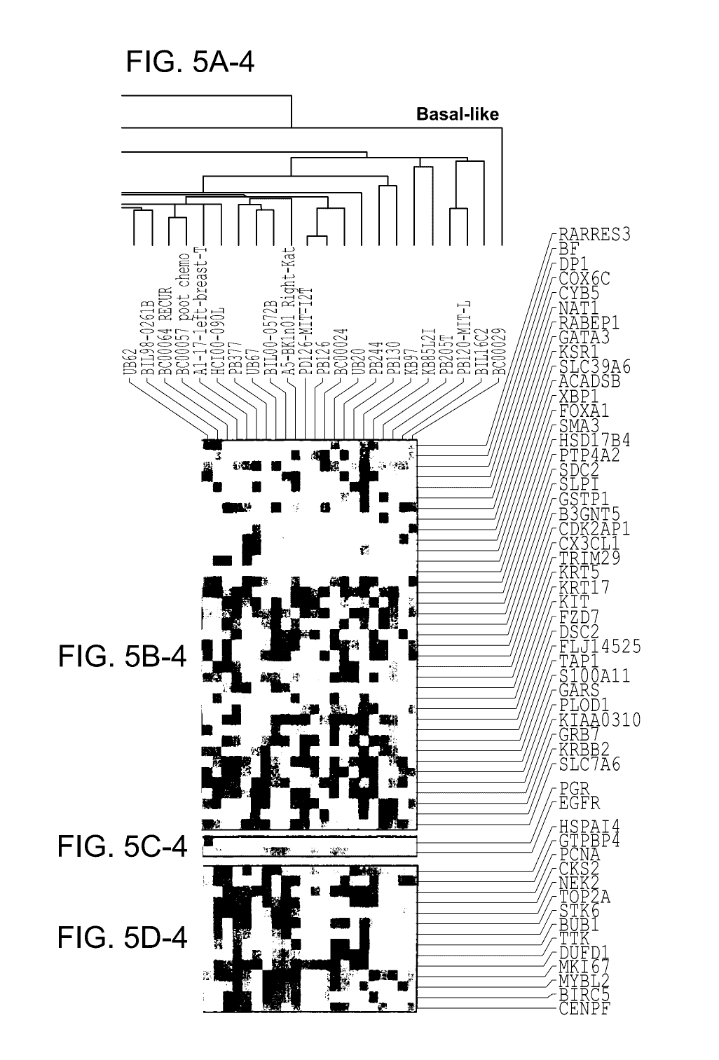

Agreement between immunohistochemistry, qRT-PCR “intrinsic” classifications, and gene expression. Fifty out of fifty-five (91%) Luminal tumors with IHC data were scored positive for ER. Conversely, 50 out of 56 (89%) tumors classified as HER2+/ER− or Basal-like were negative for ER by IHC. Cluster analysis showed that the Luminal tumors co-express ER and estrogen responsive genes such as LIV1/SLC39A6, X-box binding protein 1 (XBP1), and hepatocyte nuclear factor 3a (HNF3A/FOXA1). The gene with the highest correlation in expression to ESR1 was GATA3 (0.79, 95% CI: 0.71-0.85). It was found that the gene expression of ESR1 alone had 88% sensitivity and 85% specificity for calling ER status by IHC, and GATA3 alone showed 79% sensitivity and 88% specificity (FIG. 5A). In addition, gene expression of PgR correlated well with PR IHC status (sensitivity=89%, specificity=82%) (FIG. 5B). The data showed a very high correlation in expression between HER2/ERBB2 and GRB7 (0.91, 95% CI: 0.87-0.94), which are physically located near one another and are commonly overexpressed and DNA amplified together (Pollack et al. (1999) Nature Genetics 23:41-46; Pollack et al. (2002) Proc Natl Acad Sci USA 99:12963-12968). However, neither ERBB2 (sensitivity=91%, specificity=54%) nor GRB7 (sensitivity=52% specificity=78%) gene expression had both high sensitivity and specificity for predicting HER2 status by IHC (FIG. 5C).

Reproducibility of qRT-PCR. The run-to-run variation in Cp (cycle number determined from fluorescence crossing point) for all 56 genes (53 classifiers and 3 housekeepers) was determined from 8 runs. The median CV (standard deviation/mean) for all the genes was 1.15% (0.28%-6.55%) and 51/56 genes (91%) had a CV <2%. The reproducibility of the classification method is illustrated from the observation that replicates of the same sample (UB57A&B and UB60A&B), cluster directly adjacent to one another. Notably, the replicates were from separate RNA/cDNA preparations done on different pieces of the same tumor.

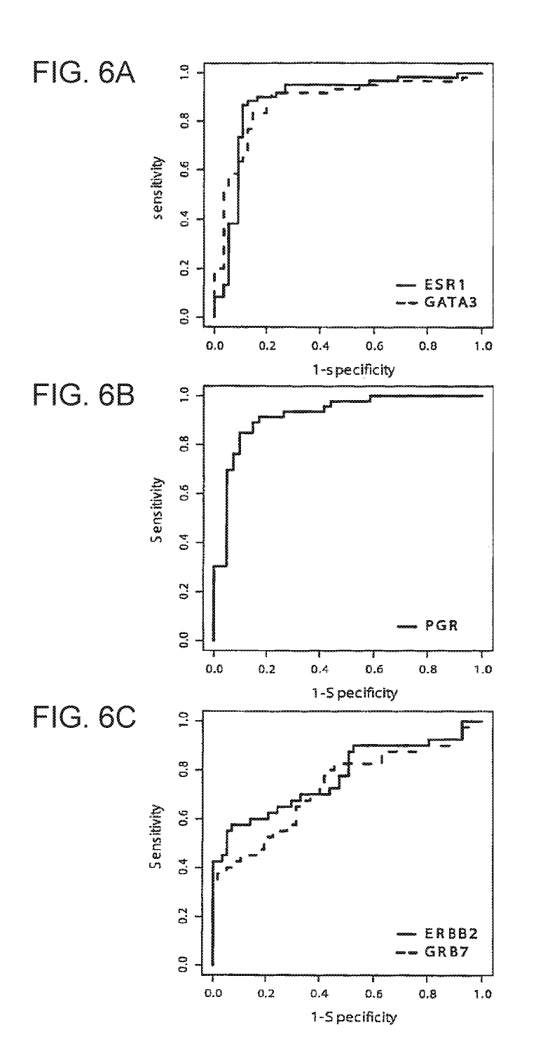

Survival Predictors. The clinical significance of individual markers and “intrinsic” subtypes were analyzed using qRT-PCR data. Patients with Luminal tumors showed significantly better outcomes for relapse-free survival (RPS) and overall survival (OS) compared to HER2+/ER− (RFS: p=0.023; OS: p=0.003) and Basal-like (RFS: p=0.065; OS: p=0.002) tumors (FIG. 6). This difference in outcome was significant for overall survival even after adjustment for stage (HER2+/ER−: p=0.043; Basal-like: p=0.001). There was no difference in outcome between patients with HER2+/ER− and Basal-like tumors. Analysis of the same cohort using standard clinical pathological information shows that stage, tumor size, node status, and ER status were prognostic for RFS and OS.

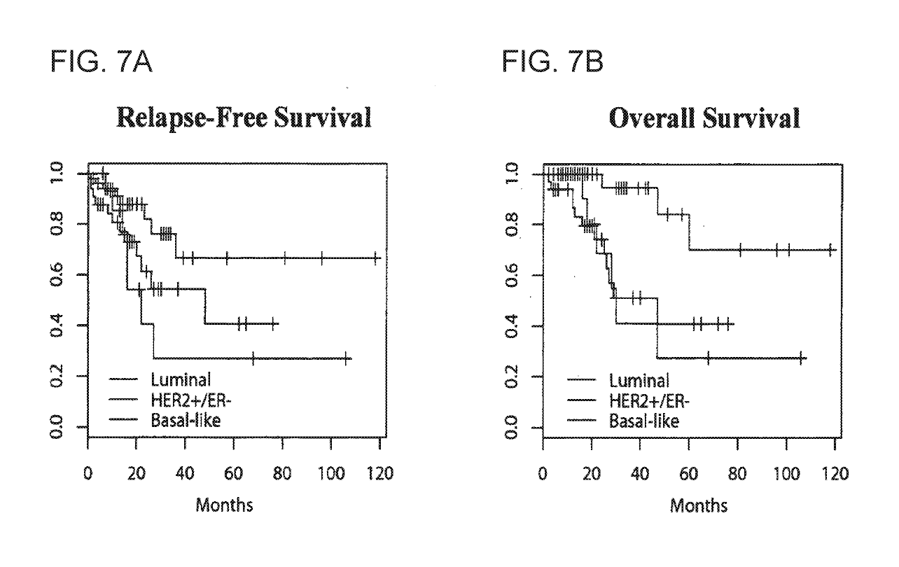

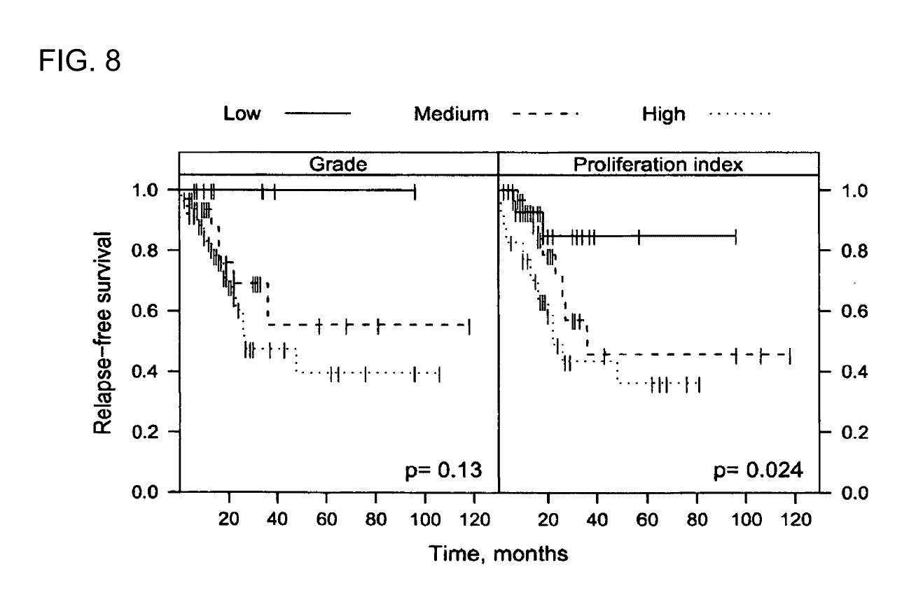

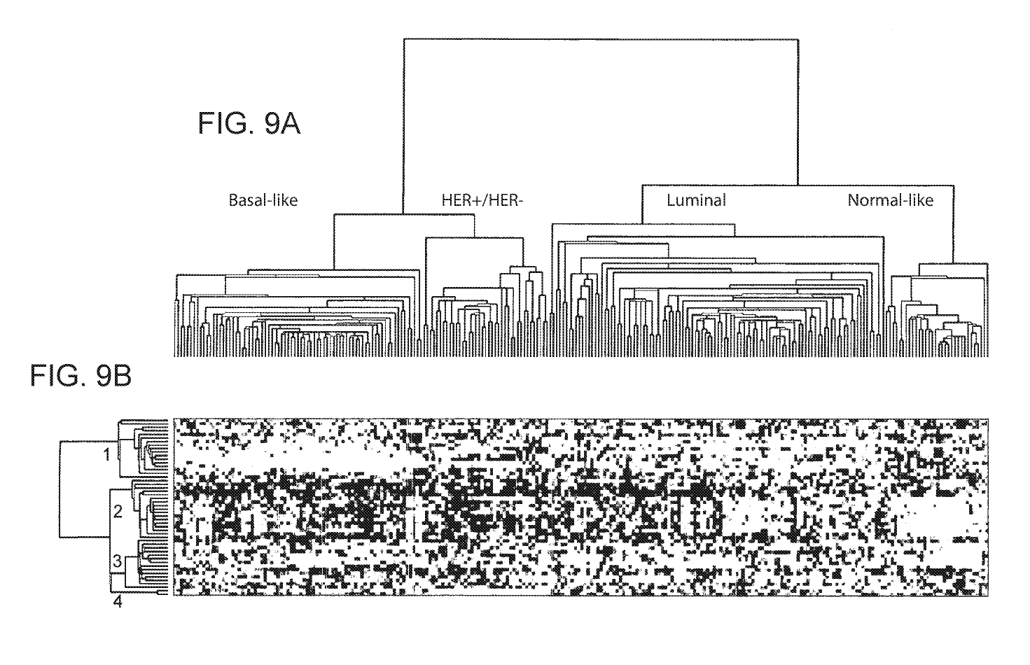

Using a Cox proportional hazards model to find biomarkers from the qRT-PCR data that predict survival, it was found that high expression of the proliferation genes GTBP4 (p=0.011), HSPA14 (p=0.023), and STK6 (p=0.027) were significant predictors of RFS independent of grade and stage (FIG. 7). The only proliferation gene significant for OS after correction for grade and stage was GTBP4 (p=0.011). Overall, the best predictor for both RFS (p=0.004) and OS (p=0.004) independent of grade and stage was SMA3 (Table 10).

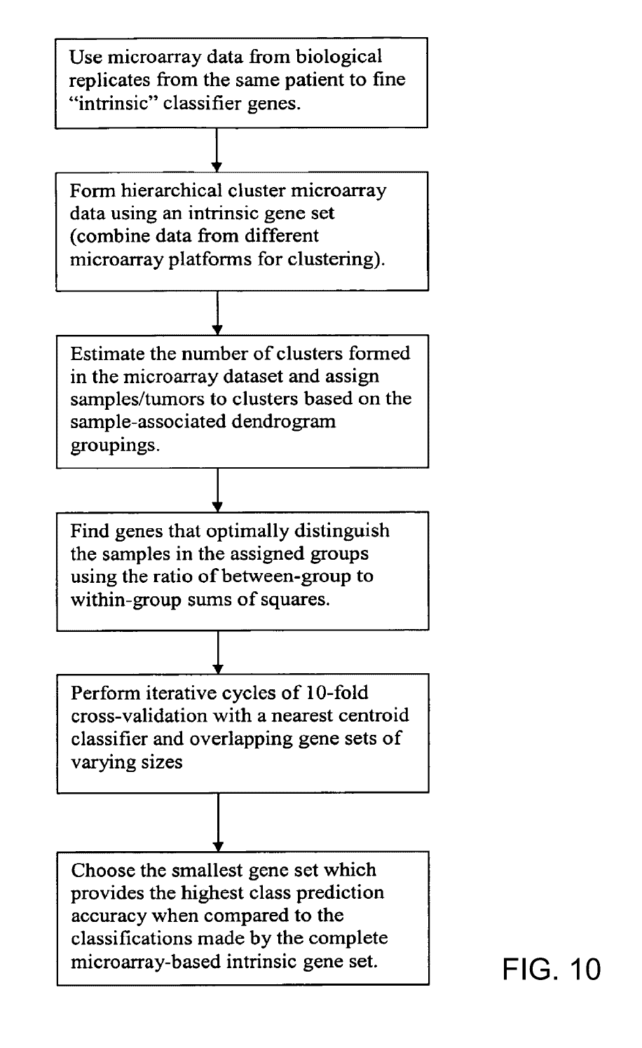

Co-clustering qRT-PCR and Microarray Data. In order to determine if qRT-PCR and microarray data could be analyzed together in a single dataset, DWD was used to combine data for 50 genes and 126 samples profiled on both platforms (252 samples total). Hierarchical clustering of these data show that 98% (124/126) of the paired samples classified in the same group and 83/126 (66%) clustered directly adjacent to their corresponding partner (FIG. 10). Thus, DNA microarray and real-time qRT-PCR can be combined into a seamless dataset without sample segregation based on platform. Overall, the correlation between microarray and qRT-PCR expression data was 0.76 (95% CI: 0.75, 0.77) before DWD and 0.77 (95% CI: 0.76, 0.78) after DWD (FIG. 5). The DWD does not significantly effect the correlation but corrects for systematic biases between the platforms.

c) Discussion

Gene expression analyses can identify differences in breast cancer biology that are important for prognosis. However, a major challenge in using genomics for diagnostics is finding biomarkers that can be reproducibly measured across different platforms and that provide clinically significant classifications on different patient populations. Using microarray data, 402 “intrinsic” genes were identified that classify breast cancers based on vastly different expression patterns. This “intrinsic” gene set was shown to provide the same classifications when applied to a completely new and ethnically diverse population. Furthermore, the microarray dataset can be minimized to 37 “intrinsic” genes, translated into a real-time qRT-PCR assay, and provide the same classifications as the larger gene set. Molecular classifications using the “intrinsic” qRT-PCR assay agree with standard pathology and are clinically significant for prognosis. Thus, biological classifications based on “intrinsic” genes are robust, reproducible across different platforms, and can be used for breast cancer diagnostics.

The greatest contribution genomic assays have made towards clinical diagnostics in breast cancer has been in identifying risk of recurrence in women with early stage disease. For instance, MammaPrint™ is a microarray assay based on the 70 gene prognosis signature originally identified by van't Veer et al. On the test set validation, the 70 gene assay found that individuals with a poor prognostic signature had approximately a 50% chance of remaining free of distant metastasis at 10 years while those with a good-prognostic signature had a 85% chance of remaining free of disease. Another assay with similar utility is Oncotype Dx (Genomic Health Inc)—a real-time qRT-PCR assay that uses 16 classifiers to assess if patients with ER positive tumors are at low, intermediate, or high risk for relapse. While recurrence can be predicted with high and low risk tumors, patients in the intermediate risk group still have variable outcomes and need to be diagnosed more accurately.

In general, tumors that have a low risk of early recurrence are low grade and have low expression of proliferation genes. Due to the correlation of proliferation genes with grade and their significance in predicting outcome, a group of 14 proliferation genes were assayed. While the classic proliferation markers TOP2A and MKI67 significantly correlated with grade in the cohort, they were not near the top of the list. Furthermore, PCNA did not significantly correlate with grade (p=0.11) in the cohort. This could result from PCR primer design or differences between RNA and protein stability. Nevertheless, the proliferation gene that was found had the highest correlation to grade was CENPF (mitosin); another commonly used mitotic marker that has been shown to correlate with grade and outcome in breast cancer (Clark et al. (1997) Cancer Res 57:5505-5508). Since tumor grade and the mitotic index have been shown to be important in predicting risk of relapse (Chia et al. (2004) J Clin Oncol 22:1630-1637; Manders et al. (2003) Breast Cancer Res Treat 77:77-84), it is not surprising that 4 (GTBP4, HSPA14, STK6/15, BUB1) out the top 5 predictors for RFS (independent of stage) were proliferation genes. The proliferation gene that was the best predictor of RFS was GTBP4, a GTP-binding protein implicated in chronic renal disease and shown to be upregulated after serum administration (i.e., serum response gene) (Laping et al. (2001) J Am Soc Nephrol 12:883-890). Overall, the best predictor for both RFS (ρ=0.004) and OS (p=0.004) independent of grade and stage was SMA3. The role of SMA3 in the pathogenesis of breast cancer is still unclear, although it has also been associated with the BCL2 anti-apoptotic pathway (Iwahashi et al. (1997) Nature 390:413-417).

### 2. Example 2

### A New Breast Tumor Intrinsic Gene List Identifies Novel Characteristics that are Conserved Across Microarray Platforms

A training set of 105 tumors were used to derive a new breast tumor “intrinsic” gene list and validated it using a combined test set of 315 tumors compiled from three independent microarray studies. An unchanging Single Sample Predictor was also used, and applied to three additional test sets. The Mrinsic/UNC gene set identified a number of findings not seen in previous analyses including 1) significance in multivariate testing, 2) that the proliferation signature is an intrinsic property of tumors, 3) the high expression of many Kallikrein genes in Basal-like tumors, and 4) the expression of the Androgen Receptor within the HER2+/ER− and Luminal tumor subtypes. The Single Sample Predictor that was based upon subtype average profiles, was able to identity groups of patients within a test set of local therapy only patients, and two independent tamoxifen-treated patient sets, which showed significant differences in outcomes. The analyses demonstrates that the “intrinsic” subtypes add value to the existing repertoire of clinical markers used for breast cancer patients. The computation approach also provides a means for quickly validating gene expression profiles using publicly available data.

Breast cancers represent a spectrum of diseases comprised of different tumor subtypes, each with a distinct biology and clinical behavior. Despite this heterogeneity, global analyses of primary breast tumors using microarrays have identified gene expression signatures that characterize many of the essential qualities important for biological and clinical classification. Using cDNA microarrays, five distinct subtypes of breast tumors arising from at least two distinct cell types (basal-like and luminal epithelial cells) were previously identified (Perou et al. 2000; Sorlie et al. 2001; Sorlie et al. 2003). This molecular taxonomy was based upon an “intrinsic” gene set, which was identified using a supervised analysis to select genes that showed little variance within repeated samplings of the same tumor, but which showed high variance across tumors (Perou et al. 2000). An intrinsic gene set reflects the stable biological properties of tumors and typically identifies distinct tumor subtypes that have prognostic significance, even though no knowledge of outcome was used to derive this gene set.

315 breast tumor samples compiled from publicly available microarray data were generated on different microarray platforms. These analyses show for the first time, that the breast tumor intrinsic subtypes are significant predictors of outcome when correcting for standard clinical parameters, and that common patterns of expression and outcome predictions can be identified when comparing data sets generated by independent labs.

A) Methods

Tissue samples, RNA preparations and microarray protocols. 105 fresh frozen breast tumor samples and 9 normal breast tissue samples were used as the training set and were obtained from 4 different sources using IRB approved protocols from each participating institution: the University of North Carolina at Chapel Hill, The University of Utah, Thomas Jefferson University and the University of Chicago. Thus, this sample set represents an ethnically diverse cohort from different geographic regions in the US with the clinical and microarray data for samples provided in Table 11. Patients were heterogeneously treated in accordance with the standard of care dictated by their disease stage, ER and HER2 status. The 105 patient training data set had a median follow up of 19.5 months, while the 315 sample combined test set had a median follow up of 74.5 months. Finally, another 16 tamoxifen-treated patient tumor samples were included that were used for the Single Sample Predictor additional test set analysis (tamoxifen-treated set #2).

Total RNA was purified from each sample using the Qiagen RNeasy Kit according to the manufacturer's protocol (Qiagen, Valencia Calif.) and using 10-50 milligram of tissue per sample. The integrity of the RNA was determined using the RNA 6000 Nano LabChip Kit and an Agilent 2100 Bioanalyzer (Agilent Technologies, Palo Alto, Calif.). The total RNA labeling and hybridization protocol used is described in the Agilent low RNA input linear amplification kit (http://www.chem.agilent.com/Scripts/PDS.asp?lPage=10003) with the following modifications: 1) a Qiagen PCR purification kit was used to clean up the cRNA and 2) all reagent volumes were cut in half. Each sample was assayed versus a common reference sample that was a mixture of Stratagene's Human Universal Reference total RNA (Novoradovskaya et al. 2004) (100 ug) enriched with equal amounts of RNA (0.3 μg each) from MCF7 and ME16C cell lines. Microarray hybridizations were carried out on Agilent Human oligonucleotide microarrays (1A-v1, 1A-V2 and custom designed 1A-v1 based microarrays) using 2 μg of Cy3-labeled Reference and 2 μg of Cy5-labeled experimental sample. Hybridizations were done using the Agilent hybridization kit and a Robbins Scientific “22k chamber” hybridization oven. The arrays were incubated overnight and then washed once in 2×SSC and 0.0005% triton X-105 (10 min), twice in 0.1×SSC (5 min), and then immersed into Agilent Stabilization and Drying solution for 20 seconds. AU microarrays were scanned using an Axon Scanner GenePix 4000B. The image files were analyzed with GenePix Pro 4.1 and loaded into the UNC Microarray Database at the University of North Carolina at Chapel Hill (https://genome.unc.edu/) where a Lowess normalization procedure was performed to adjust the Cy3 and Cy5 channels (Yang et al. 2002). All primary microarray data associated with this study are available at https://genome.unc.edu/pubsup/breastTumor/ and have been deposited into the GEO (http://www.ncbi.nlm.nih.gov/geo/) under the accession number of GSE1992, series GSM34424-GSM34568.

Intrinsic gene set analysis. A new breast tumor intrinsic gene set was derived, called the “Intrinsic/UNC” list using 105 patients (146 total arrays) and 15 repeated tumor samples that were different physical pieces (and RNA preparations) of the same tumor, 9 tumor-metastasis pairs and 2 normal sample pairs (26 paired samples in total, Table 11). This sample size was chosen based upon Basal-like, Luminal A, Luminal B, HER2+/ER−, and Normal-like samples, which occur at a frequency of 15%, 40%, 15%, 20%, and 10%, respectively; and it was estimated that most clinically relevant classes would constitute at least 10% of the affected population, and it was hoped to acquire at least 10 samples from each class in the new data set. Therefore, a sample size of 100 tumors was deemed adequate to identify most classes that might be present in breast cancer patients.

The background subtracted, Lowess normalized Iog2 ratio of Cy5 over Cy3 intensity values were first filtered to select genes that had a signal intensity of at least 30 units above background in both the Cy5 and Cy3 channels. Only genes that met these criteria in at least 70% of the 146 microarrays were included for subsequent analysis. Next, an “intrinsic” analysis was performed as described in Sorlie et al. 2003 (Sorlie et al. 2003) using the 26 paired samples and 86 additional microarrays. An intrinsic analysis identifies genes that have low variability in expression within paired samples and high variability in expression across different tumors; for an intrinsic analysis, each gene receives a score that is the average “within-pair variance” (the average square before/after difference), as well as the “between-subject variance” (the variance of the pair averages across subjects). The ratio D=(within-pair variance)/(between-subject variance) was then computed, and those genes with a small value of D (i.e. cut-off) declared to be “intrinsic”. The choice of a value of D was set at one standard deviation below the mean intrinsic score of all genes. This analysis resulted in the selection of 1410 microarray elements representing 1300 genes. In order to obtain an estimate of the number of false-positive intrinsic genes, the sample labels were permuted to generate 26 random pairs and 86 non-paired samples. This permutation was performed 100 times and the intrinsic scores were calculated for each. These permuted scores were used to determine a threshold on the intrinsic score corresponding to a false discovery rate less than 1%. The selected threshold resulted in 1410 microarray features being called significant with a FDR=0.3% and the 90th percentile FDR=0.5%. (See Tusher et al. for a complete description of this calculation (Tusher et al. 2001)).

These 1410 microarray elements were then used to perform a two-way average linkage hierarchical cluster analysis using a centered Pearson correlation metric and the program “Cluster” (Eisen et al. 1998), with the data being displayed relative to the median expression for each gene (i.e. median centering of the rows/genes). The cluster results were then visualized using “Treeview”.

Combined test set analysis. The two-color DNA microarray data sets of Sorlie et al. 2001 and 2003 van't Veer et al. and Sotiriou et al. (Sotiriou et al. 2003) were each downloaded from the internet and pre-processed similarly. Briefly, pre-processing included Iog2 transformation of the R/G ratio and then Lowess normalization of the data set (Yang et al. 2002 J. Next, missing values were imputed using the k-NN imputation algorithm described by Troyanskaya et al. (Troyanskaya et al. 2001). Gene annotation from each dataset was translated to UniGene Cluster IDs (UCTD) using the SOURCE database (Diehn et al. 2003), which gave a common gene set of approximately 2800 genes that were present across all four data sets. UniGene was chosen because a majority of the identifiers from each dataset could be easily mapped to a UniGene identifier (Build 161). Multiple occurrences of a UCDD were collapsed by taking the median value for that E) within each experiment and platform. Next, Distance Weighted Discrimination was performed in a pair-wise fashion by first combining the Sorlie et al. data set with the Sotiriou et al. data set, and then combining this with the van't Veer et al. data to make a single data set. hi the final step of pre-processing, each individual experiment (microarray) was normalized by setting the mean to zero and its variance to one. The data for 306 of the 1300 Intrinsic/UNC genes was present in the combined test set and was used in a two-way average linkage hierarchical cluster analysis across the set of 315 microarrays as described above.

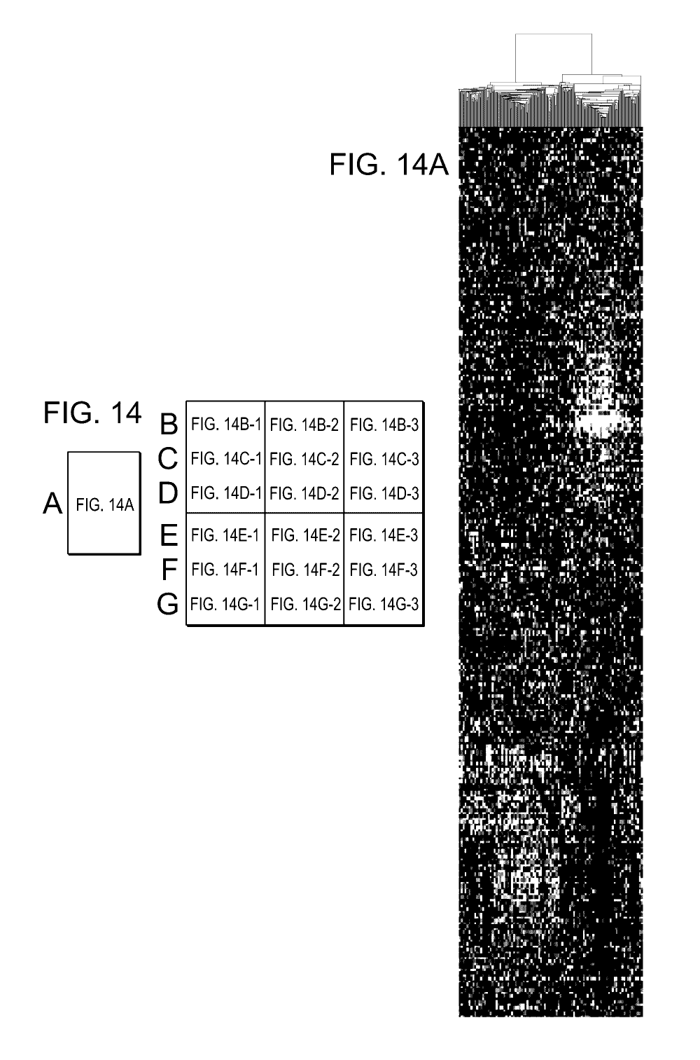

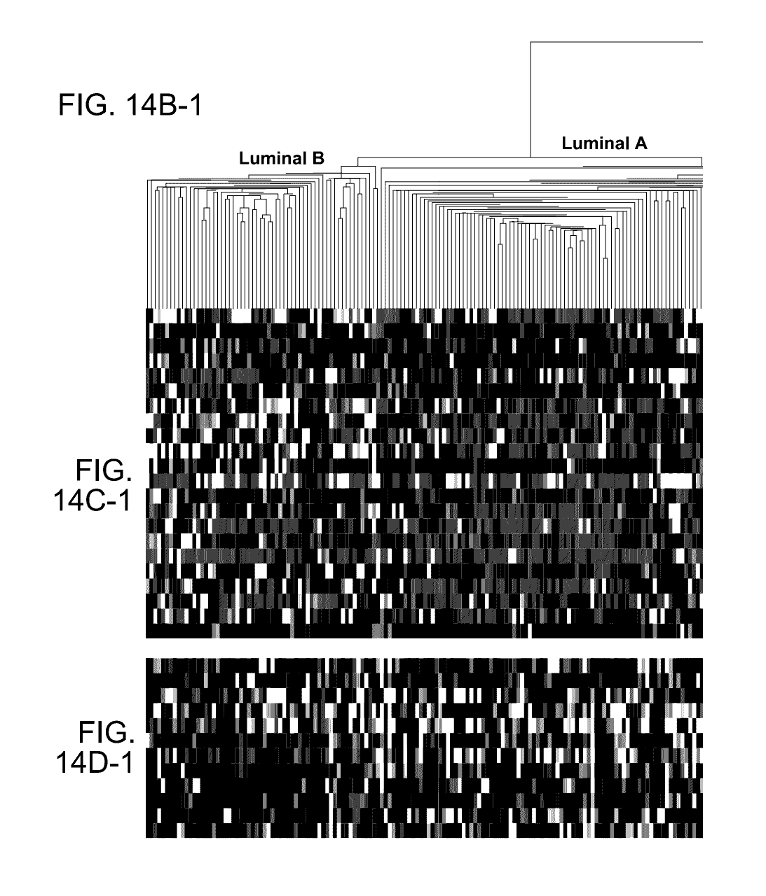

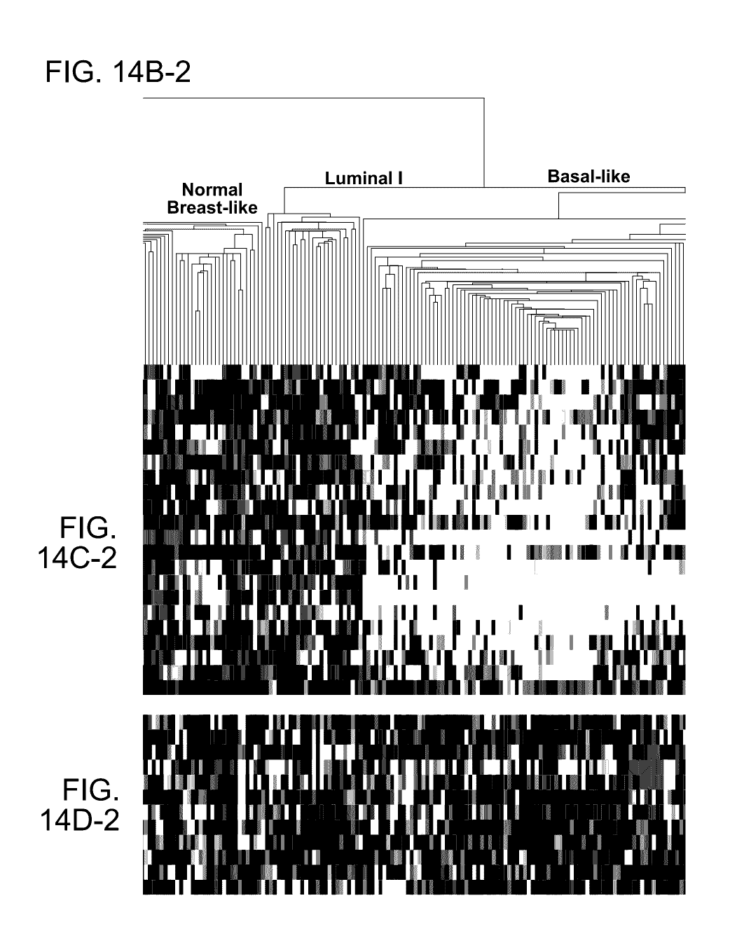

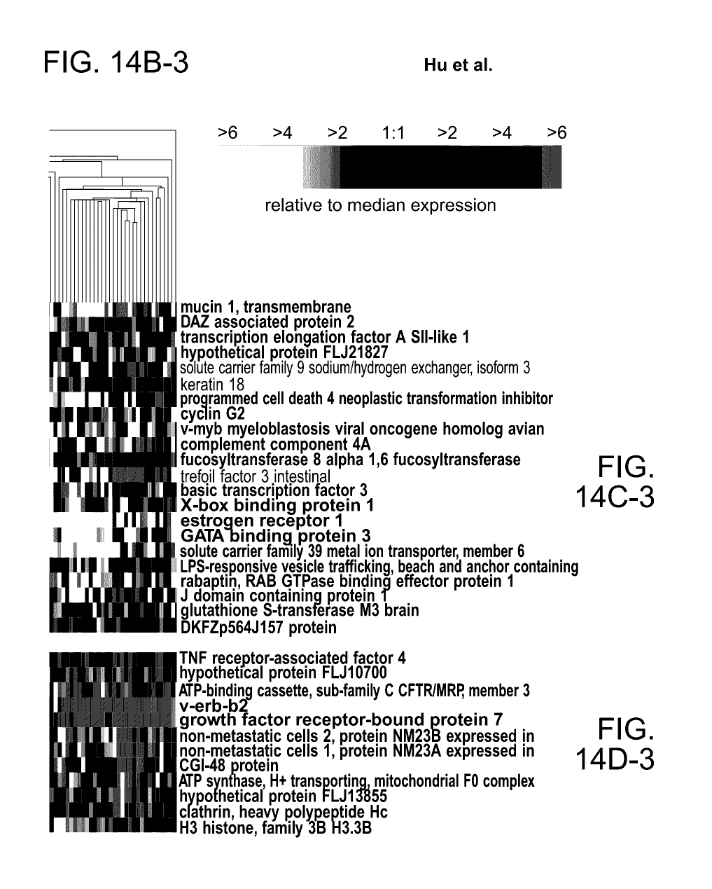

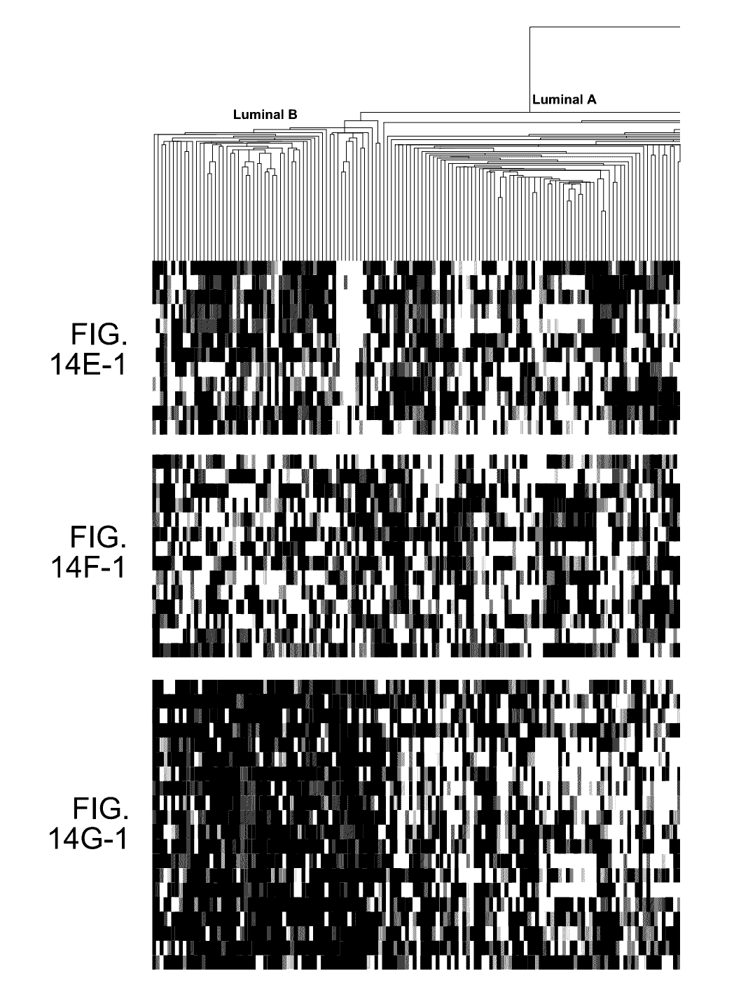

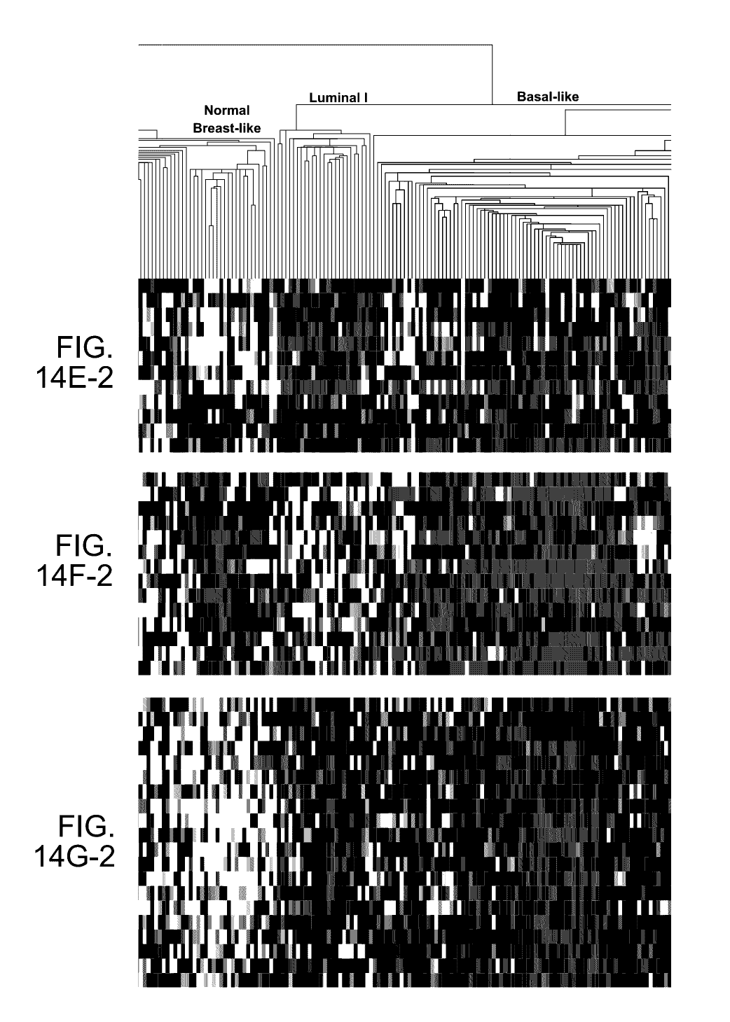

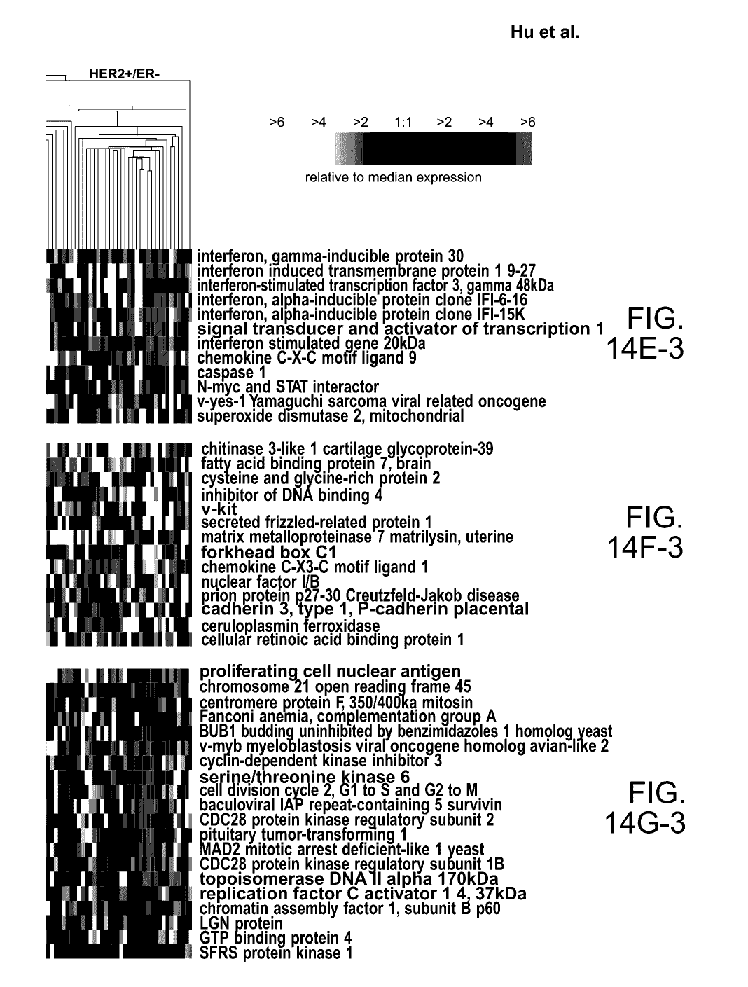

Single Sample Predictor. The Single Sample Predictor/SSP is based upon the Nearest Centroid method presented in (Hastie et al. 2001). More specifically, the combined test set was utilized, and 306 Intrinsic/UNC gene set hierarchical cluster presented in FIG. 14, as the starting point to create five Subtype Mean Centroids. A mean vector (centroid) for each of the five intrinsic subtypes (LumA, LumB, HER2+/ER−, Basal-like and Normal Breast-like) was created by averaging the gene expression profiles for the samples clearly assigned to each group (which limited the analysis to 249 samples total); the hierarchical clustering dendrogram in FIG. 14 were used as a guide for deciding those samples to group together. Next, using the 249 samples and 306 genes as a new training set (see FIG. 11), the SSP was applied back onto this data set (only the 249 samples) using Spearman correlation (which will calculate a training set error rate) and assigned a sample to the subtype to which it was most similar. This analysis showed 92% concordance with the clustering based subtype assignments.

Three additional test data sets were then analyzed: First the 60 sample data set of Ma et al. (Ma et al. 2004) was taken, which is an already pre-processed data set of Iog2 transformed ratios (GEO GSE1379), and performed a DWD correction using the 278 genes that were in common between the Ma et al. data set and the set of 306 Intrinsic/UNC genes used in the SSP. The SSP was applied to the 60 Ma et al. samples and, using Spearman correlation, each of the 60 samples were assigned to an intrinsic subtype based upon the highest correlation value to a centroid. Next, 220 samples from Chang et al. (Chang et al. 2005) were analyzed and 16 additional samples from UNC that were not used in the training set. The 220 samples represent an extension of the sample set presented in van't Veer et al. (van't Veer et al. 2002), and the combination of these two are the data used in van de Vijver et al. (van de Vijver et al. 2002). Each sample was column-standardized and then performed DWD to combine the 249 SSP samples (306 intrinsic genes) with the 220 samples from Chang et al. and the 16 UNC additional test set samples. Next, each sample's correlation to each centroid was calculated using a Spearman correlation and a sample was assigned to the centroid it was closest to, and the test set was then split into a local only therapy test set, and a tamoxifen-treated test set. Finally, the SSP was applied to the 105 sample original training set after DWD normalization.

Survival analyses. Univariate Kaplan-Meier analysis using a log-rank test was performed using WinSTAT for excel (R. Fitch Software). Standard clinical pathological parameters of age (in decades), node status (positive vs. negative), tumor size (categorical variable of T1-T4), grade (I vs. II and I vs. III), and ER status (positive vs. negative) were tested for differences in RFS, OS and DSS using a proportional hazards regression model. The likelihood ratio test was used to test for equality of the hazard functions among the intrinsic classes after adjusting for the covariates listed above. For the intrinsic subtype analyses, the coding was such that Lum A was the reference group to which the other classes were compared. SAS (SAS Institute Inc., SAS/STAT User's Guide, Version 8, 1999, Cary, N.C.) was used for proportional hazards modeling.

Immunohistochemistry. Five micron sections from formalin-fixed, paraffin-embedded tumors were cut and mounted onto Probe On Plus slides (Fisher Scientific). Following deparaffinization in xylene, slides were rehydrated through a graded series of alcohol and placed in running water. Endogenous peroxidase activity was blocked with 3% hydrogen peroxidase and methanol. Samples were steamed for antigen retrieval with 10 mM citrate buffer (pH 6.0) for 30 min. Following protein block, slides were incubated with biotinylated antibody for the Androgen Receptor (Zymed, 08-1292) and incubated with streptavidin conjugated HRP using Vectastain ABC kit protocol (Vector Laboratories). 3,3′-diaminobenzidine tetrahydrochloride (DAB) chromogen (the substrate) was used for the visualization of the antibody/enzyme complex. Slides were counterstained with hematoxylin (Biomeda-MIO) and examined by light microscopy.

B) Results

Overview. The goals were to create a new breast tumor intrinsic list and validate this list using multiple test data sets so that new biology could be identified, and the clinical significance of “intrinsic” classifications shown. A new intrinsic list was created using paired samples that were similarly treated (note that these were different “intrinsic” pairs than previously used since they were not before and after therapy pairs). In deriving the “new” list microarrays containing many more thousands of genes than was used before were used. A diagram representing the flow of data sets used here, and the different analysis methods, is presented in FIG. 11. First, a new 1300 gene “Intrinsic/UNC” list was created using 26 paired samples and a “training set” of 105 patients. Second, a large “combined test set” of 315 samples was created by combining three publicly available data sets. A reduced version of the Intrinsic/UNC gene set (reduced to an overlapping set of 306 genes) was applied onto this pure test set and show significance in a multivariate analysis. Finally, using the “combined test set”, a Single Sample Predictor (SSP) was created from the subtype average profiles (i.e. centroids) and assign subtype designation onto three “additional test sets”. Thus, the “combined test set” becomes the training set for the SSP, which is then used to predict subtype, and ultimately outcome, on the “additional test sets”.

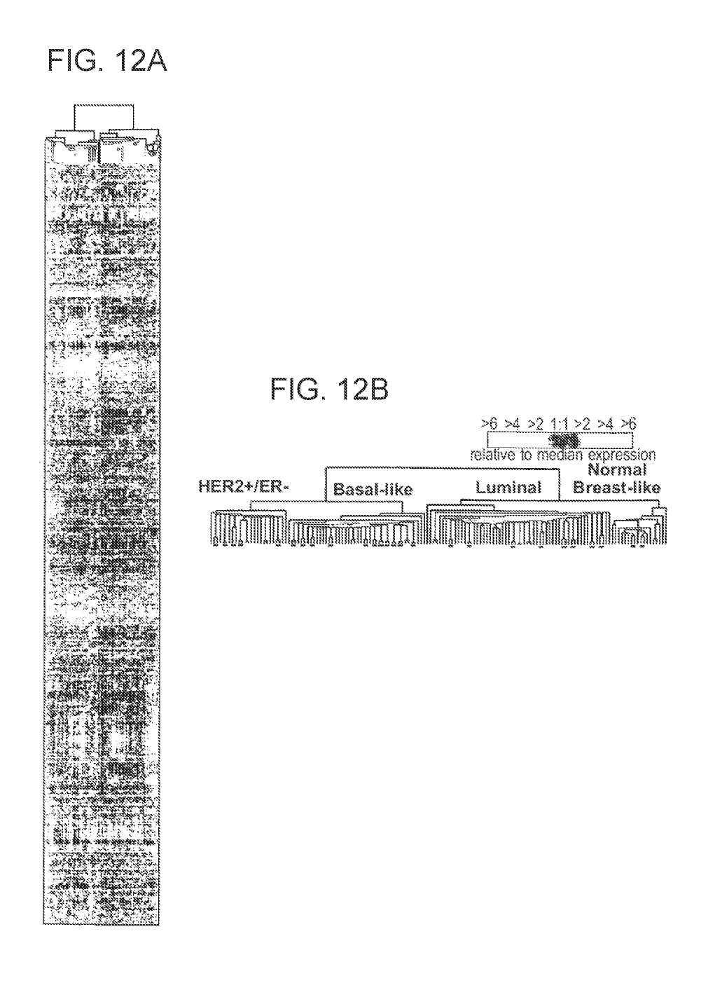

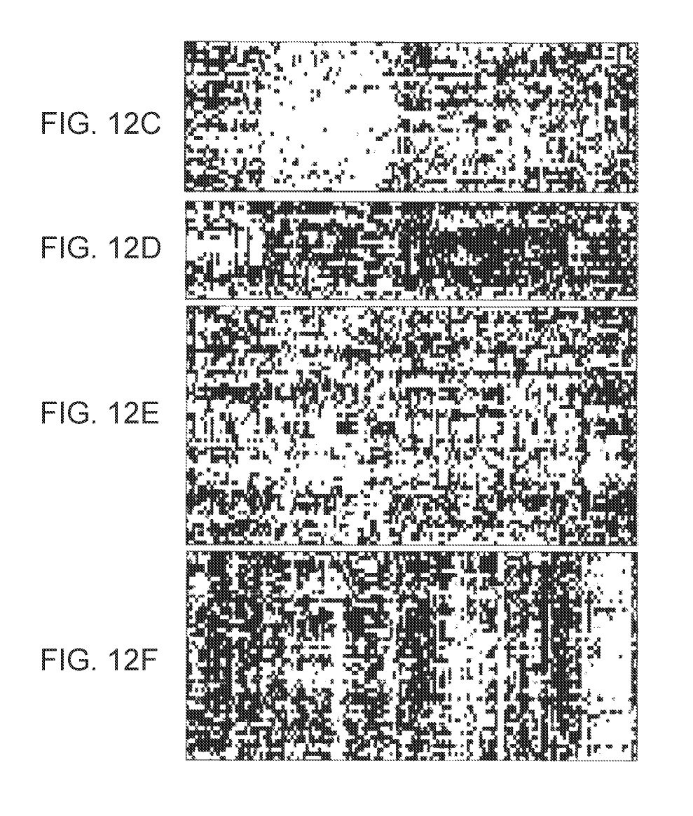

Identification of the Intrinsic/UNC gene set. A new breast tumor intrinsic gene set was created, called the “Intrinsic/UNC” list, using 26 paired samples comprised of 15 paired primary tumors that were different physical pieces (and RNA preparations) of the same tumor, 9 primary tumor-metastasis pairs, and 2 normal breast sample pairs. In total, 105 biologically diverse breast tumor specimens and 9 normal breast samples (146 microarrays, see Table 11) were assayed on Agilent oligo DNA microarrays representing 17,000 genes (GEO accession number GSE 1992). This intrinsic analysis identified 1410 microarray elements that represented 1300 genes. When this new gene list was used in a two-way hierarchical clustering analysis on the training set (FIG. 12), the experimental sample dendrogram (FIG. 12B) showed four groups corresponding to the previously defined HER2+/ER−, Basal-like, Luminal and Normal Breast-like groups (Perou et al. 2000). AU 26 tumor pairs were paired in this clustering analysis, including the 5 primary tumor-local metastasis pairs and the 4 distant metastasis pairs (FIG. 12); thus, the individual portraits of tumors are maintained even in their metastasis samples (Weigelt et al. 2003).

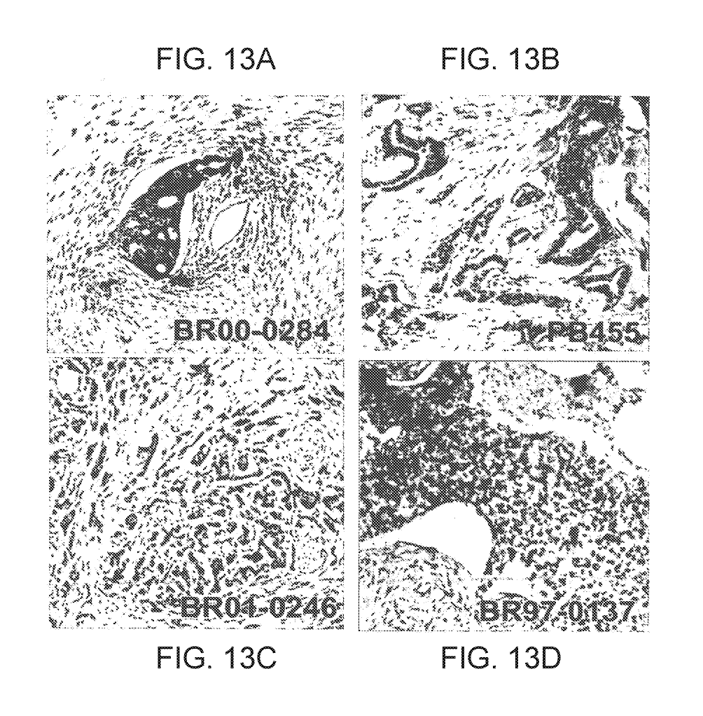

The biology of the intrinsic subtypes is rich and extensive, and the current analysis identified new biologically important features. A HER2+ expression cluster was observed that contained genes from the 17qI 1 amplicon including HER2/ERBB2 and GRB7 (FIG. 12D). The HER2+ expression subtype (pink dendrogram branch in FIG. 12B) was predominantly ER-negative (i.e. HER2+/ER−), but showed expression of the Androgen Receptor (AR) gene. To determine if this finding extended to the protein level, immunohistochemistry for AR was performed, and it was confirmed that the HER2+/ER− and many Luminal tumors, expressed AR at moderate to high levels (FIG. 13); in some cases, high nuclear expression was observed (FIG. 13B).

A Basal-like expression cluster was also present and contained genes characteristic of basal epithelial cells such as SOX9, CK17, c-KIT, FOXC1 and P-Cadherin (FIG. 13E). These analyses extend the Basal-like expression profile to contain four Kallikrein genes (KLK5-8), which are a family of serine proteases that have diverse functions and proven utility as biomarkers (e.g. KLK3/PSA); however, it should be noted that KLK3/PSA was not part of the basal profile. Finally, a Luminal/ER+ cluster was present and contained ER, XBP1, FOXA1 and GAT A3 (FIG. 12C). GATA3 has recently been shown to be somatically mutated in some ER+ breast tumors (Usary et al. 2004), and some of the genes in FIG. 12C are GAT A3-regulated (FOXA1, TFF3 and AGR2). In addition, the Luminal/ER+ cluster contained many new biologically relevant genes such as AR (FIG. 12C), FBP1 (a key enzyme in gluconeogenesis pathway) and BCMP11.

The subtype defining genes from this analysis showed similarity to the previous breast tumor intrinsic lists (i.e. Intrinsic/Stanford) described in (Perou et al. 2000; Sorlie et al. 2003), except there was a significant increase in gene numbers likely due to the increased number of genes present on the current microarrays, and another significant difference was that the new Intrinsic/UNC list contained a large proliferation signature (FIG. 12F) (Perou et al. 1999; Chung et al. 2002; Whitfield et al. 2002). The inclusion of proliferation genes in the Intrinsic/UNC gene set, but not in the previous Intrinsic/Stanford lists, is likely due to the fact that the Intrinsic/Stanford lists were based upon before and after chemotherapy paired samples of the same tumor, while the Intrinsic/UNC list was based upon identically treated paired samples. This finding suggests that tumor cell proliferation rates did vary before and after chemotherapy, and that proliferation is a reproducible feature of a tumor's expression profile. Thus, the new Intrinsic/UNC list likely encompasses most features of the previous lists, adds new genes to each subtype's defining gene set and adds a biological and clinically relevant feature that is the proliferation signature.

Combined test set analysis. Another difference between the intrinsic subtypes found in the 105 sample training data set versus those presented in Sorlie et al. 2001 and 2003 (Sorlie et al. 2001; Sorlie et al. 2003), was that the training set did not have a clear Luminal B (LumB) group as determined by hierarchical clustering analysis. The lack of a LumB group in the training set cluster analysis could be due to few LumB tumors being present in this data set, an artifact of the clustering analysis, or the lack of LumB defining genes in the Intrinsic/UNC gene list. To address this question, a “combined test set” of 315 breast samples was made (311 tumors and 4 normal breast samples) that was a single data set created by combining together the data from Sorlie et al. 2001 and 2003 (cDNA microarrays), van't Veer et al. 2002 (custom Agilent oligo microarrays) and Sotiriou et al. 2003 (cDNA microarrays).

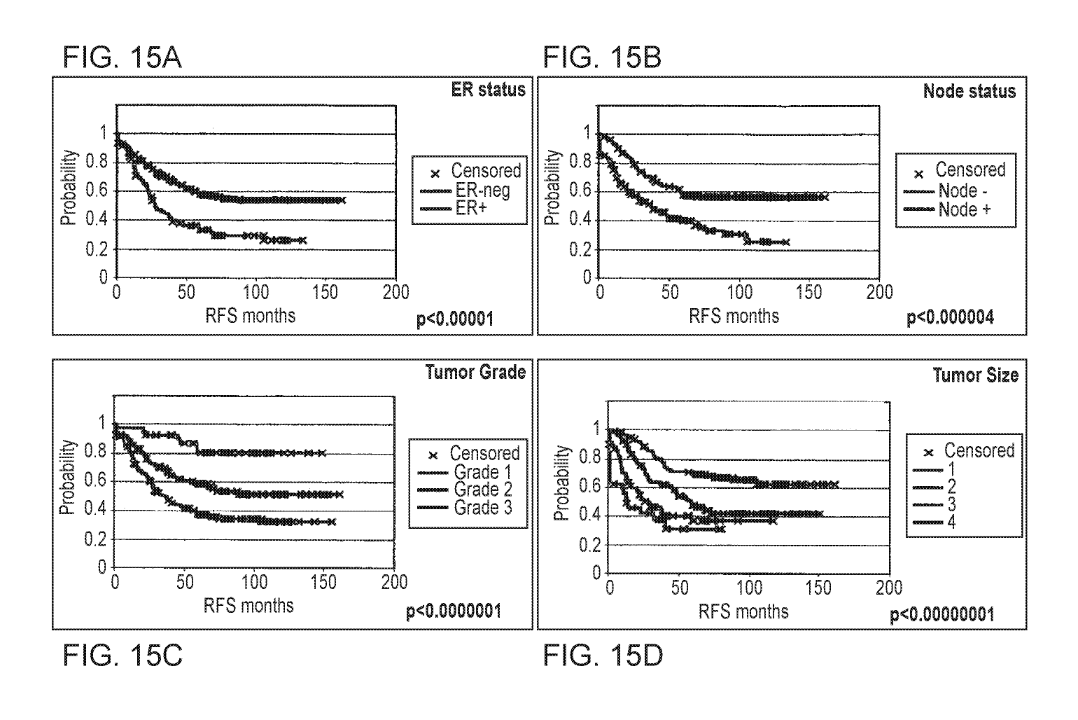

A single data table of these three sets was created by first identifying the common genes present across all four microarray data sets (2800 genes). Next, Distance Weighted Discrimination (DWD) was used to combine these three data sets together (Benito et al. 2004); DWD is a multivariate analysis tool that is able to identify systematic biases present in separate data sets and then make a global adjustment to compensate for these biases. Finally, it was determined that 306 of the 1300 unique Intrinsic/UNC genes were present in the combined test set. FIG. 14 shows the 315 sample combined test set and the 306 Intrinsic/UNC genes in a two-way hierarchical cluster analysis (see Supplementary FIG. 12 for the complete cluster diagram). As expected, this analysis identified the same expression patterns seen in FIG. 12 and more. For example, there was a Luminal/ER+ cluster containing ER, GATA3 and GAT A3-regulated genes (FIG. 14C), a HER2+ cluster (FIG. 15D), a Basal-like cluster (FIG. 14F) and a prominent proliferation signature (FIG. 14). The sample-associated dendrogram (FIG. 14B) showed the major subtypes seen in Sorlie et al. 2003 including a LumB group, and a potential new tumor group (Luminal T) characterized by the high expression of Interferon (IFN)-regulated genes (FIG. 14E). The IFN-regulated cluster contained STAT1, which is likely the transcription factor that regulates expression of these IFN-regulated genes (Bromberg et al. 1996; Matikainen et al. 1999). The IFN cluster was one of the first expression patterns to be identified in breast tumors (Perou et al. 1999), and since has been linked to positive lymph node metastasis status and a poor prognosis (Huang et al. 2003; Chung et al. 2004). The effectiveness of the DWD normalization is evident upon close examination of the sample associated dendrogram, which shows that every subtype is populated by samples from each data set (i.e. significant inter-data set mixing).

Even though there was limited overlap between the new Intrinsic/UNC list and the Intrinsic/Stanford list of Sorlie et al. 2003 (108 genes in common), there was high agreement in sample classification. For example, it was found 85% concordance in subtype assignments for the 416 tumor data set (combined samples from training and combined test set) that were analyzed independently using the Intrinsic/Stanford and Intrinsic/UNC lists, and both lists showed significance in univariate survival analyses (data not shown). This analysis suggests that, even though the exact constituent genes may vary, the different lists are tracking the same phenotypes and the same “portraits” are seen. However, since the Intrinsic/UNC list contained many more genes and a biologically relevant pattern of expression not seen in the Intrinsic/Stanford lists (i.e. proliferation signature), therefore, it can be more biologically representative of breast tumors. The Intrinsic/UNC list can also be more valuable because it provides a larger number of genes for performing across data set analyses and thus, classifications made across different platforms are less susceptible to artifactual groupings as a result of gene attrition.

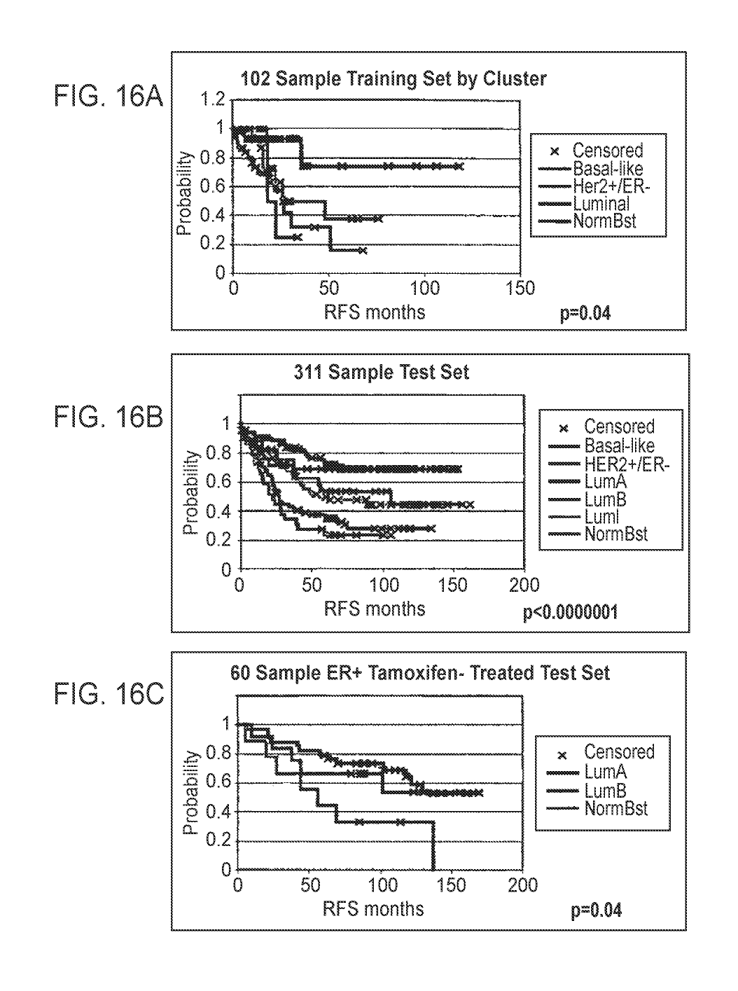

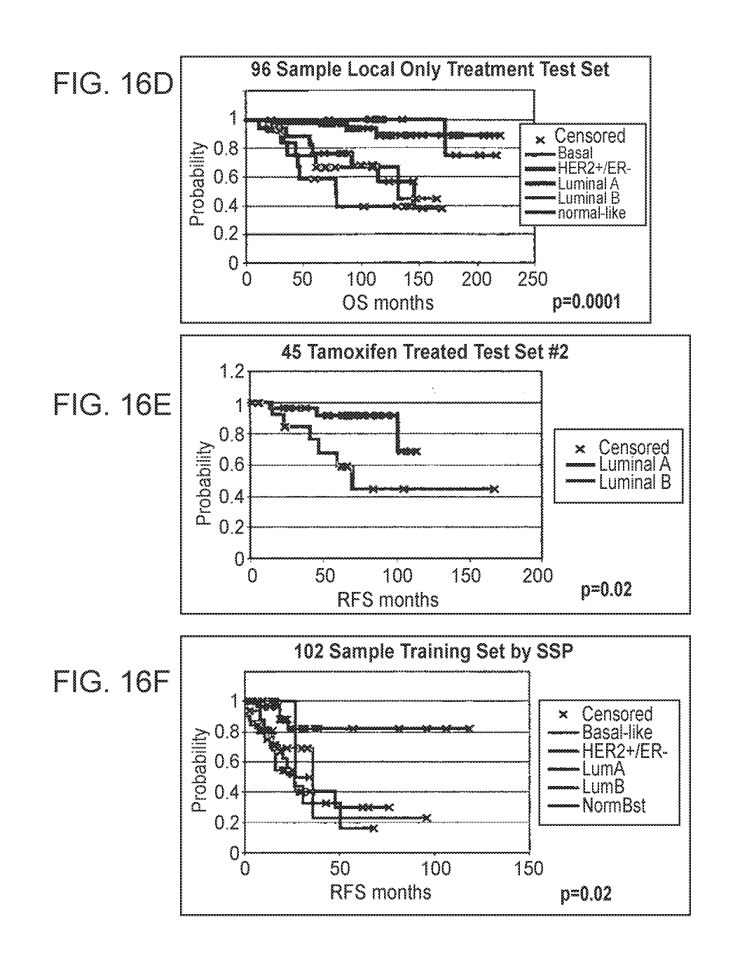

Multivariate analyses. In the training set and combined test set, the standard clinical parameters of ER status, node status, grade, and tumor size were all significant predictors of Relapse-Free Survival (RFS, where an event is either a recurrence or death) using univariate Kaplan-Meier analysis (FIG. 15 for combined test set analysis). In addition, the Intrinsic/UNC gene set identified tumor groups/subtypes that were predictive of RFS on both the training (FIG. 16A) and combined test set (FIG. 16B). As before, the Luminal group had the best outcome and the HER2+/ER− and Basal-like groups had the worst. The Intrinsic/UNC gene list was also predictive of Overall Survival (OS) on the training and combined test set. As previously seen, patients of the LumB classification showed worse outcomes that LumA, despite being clinically ER+ tumors (FIG. 16B). Finally, the new class of Lum1 showed similar outcomes to LumB, and both showed elevated proliferation rates when compared to LumA tumors (FIG. 14G).

When the five standard clinical parameters were tested on the 315 sample combined test set using a proportional hazards regression model and RFS, OS or Disease-Specific Survival (DSS) as endpoints, tumor size, grade and ER status were the significant predictors with node status being close to significant (p=0.06-0.07); however, node status was still prognostic in a univariate analysis (FIG. 15B). The next objective was to test for differences in survival among the intrinsic subtypes on the combined test set after adjusting for the clinical covariates of age, ER, node status, grade and tumor size. The approach used was a proportional hazards regression model for RFS (or time to distant metastasis for the van't Veer et al. samples), OS and DSS (which was limited to the Sorlie et al. and Sotiriou et al. data sets). P-values of 0.05 (RFS), 0.009 (OS) and 0.04 (DSS) were obtained when the intrinsic subtypes were tested in a model that included the clinical covariates, which showed that the classifications have significantly different hazard functions, and thus, different survival curves after taking into account (or adjusting for) the effects of age, node status, size, grade, and ER status (Table 11, example for RFS). In this analysis, the Basal-like, LumB and HER2+/ER− subtypes were significantly different from the LumA group (the reference group), while Lum1 was not. Similar findings were also obtained for the other endpoints except for the LumB subtype, which was not significantly different from LumA in OS (p=0.36) or DSS (p=0.08).

Single Sample Predictions using three additional test sets. A major limitation of using hierarchical clustering as a classifications tool, is its' dependence upon the sample/gene set used for the analysis (Simon et al. 2003). That is, new samples cannot be analyzed prospectively by simply adding them to an existing dataset because it may alter the initial classification of a few previous samples. If an assay is going to be used in the clinical setting, it must be robust and unchanging. To address this concern, a Single Sample Predictor (SSP) was developed using the “combined test set” and its 306 Intrinsic/UNC genes (See FIG. 11); the SSP is based upon “Subtype Mean Centroids” and a nearest centroid predictor (Hastie et al. 2001) (see Methods). For the SSP, an intrinsic subtype average profile (centroid) was created for each subtype using the combined test set presented in FIG. 14, and then a new sample is individually compared to each centroid and assigned to the subtype/centroid that it is the most similar to using Spearman correlation. Using this method, an intrinsic subtype can be assigned to any sample, from any data set, one at a time.

Using the combined test set, five centroids representing the LumA, LumB, Basal-like, HER2+/ER− and Normal Breast-like groups were created). The SSP was tested on three “additional test sets”, the first of which was the Ma et al. data set of ER+ patients that were homogenously treated with tamoxifen (Ma et al. 2004). Using the 60 whole tissue samples of Ma et al., the SSP called 2 Basal-like, 2 HER2+/ER−, 12 Normal Breast-like, 34 LumA, and 9 LumB. Since this patient set had RFS data, the SSP classifications were tested in terms of outcomes (the 2 Basal-like and 2 HER2+/ER− samples by SSP analysis were excluded). The SSP assignments were a significant predictor for this group of adjuvant tamoxifen treated patients (p=0.04, FIG. 16C).

Next, the SSP was applied onto a 96 sample test set of local only (surgery) treated patients from Chang et al. (Chang et al. 2005), which showed highly significant results (FIG. 16D, p=0.0006). The final additional test set analyzed was a second adjuvant tamoxifen-treated patient set created by combining similarly treated patients from Chang et al. 2005 plus 16 patients from UNC (which were not included within the 105 patient training data set); for the 45 patient tamoxifen treated data set #2, the SSP called 3 Normal-like, 2 Basal-like and 2 HER2+/ER−, and these samples were excluded from the survival analyses. Again, the SSP-based assignments were a statistically significant predictor of outcomes (FIG. 16E for tamoxifen-treated set #2, p=0.02). Finally, if the SSP was applied back onto the original training data set of 105 samples, it was noted that 17 tumors were called LumB (FIG. 12) and that the survival analysis showed that these tumors did show a poor outcome (FIG. 16F, p=0.02). Thus, the SSP that was based upon hundreds of samples, was able to define clinically relevant distinctions that the hierarchical clustering analysis of 105 samples missed, which further demonstrates the usefulness and objectivity of the SSP.

C) Discussion

This study identified a number of new biologically relevant “intrinsic” features of breast tumors and methods that are important for the microarray community. These new biological features include the 1) demonstration that proliferation is a stable and intrinsic feature of breast tumors, 2) identification of the high expression of many Kallikrein genes in Basal-like tumors, and 3) demonstration that there are multiple types of “HER2-positive” tumors; the HER2-positive tumors falling into the “HER2+/ER−” intrinsic subtype were also shown to associate with the expression of the Androgen Receptor, while those not falling into this group were present in the LumB or LumI subtypes and usually showed better outcomes. relative to the HER2+/ER− tumors. Recent studies in prostate cancer have shown that HER2 signaling enhances AR signaling under low androgen levels (Mellinghoff et al. 2004). When this finding is coupled to the observation that some HER2+/ER− tumors showed nuclear AR expression (FIG. 13B), this suggests that active AR signaling maybe occurring and that anti-androgen therapy can be helpful in these HER2+(i.e. amplified) and AR+ patients.

Microarray studies are often criticized for a lack of reproducibility and limited validation due to small sample sizes (Simon et al. 2003; Ioannidis 2005). By using DWD, multiple microarray data sets have been combined together to create a single and large combined test set, and it has been shown that the same “intrinsic” patterns can be identified in different data sets in a coordinated analysis, even though entirely different patient populations were investigated on different microarray platforms. The analysis of the 315 sample combined test set showed that the “intrinsic” subtypes based upon the Intrinsic/UNC list, were independent prognostic variables, and thus, were providing new clinical information.

To be of routine clinical use, a gene expression-based test must be based upon an unchanging assay that is capable of making a prediction on a single sample. Therefore, a Single Sample Predictor/SSP was created that was able to classify samples from three additional test sets of similarly treated patients. In particular, the new Intrinsic/UNC list and the SSP, recapitulated the finding that the intrinsic subtypes are truly prognostic on a test set of local only treated patients (FIG. 16D), and it was shown on two additional test sets that LumB patient fair worse than LumA patient in the presence of tamoxifen (FIGS. 16C and 16E). It should be noted that the distinction of LumA versus LumB closely mirrors the “Recurrence Score” predictor of Paik et al. (Paik et al. 2004), where outcome predictions for tamoxifen-treated ER+ tumors were stratified based mostly on the expression of genes in the HER2-amplicon (HER2 and GRB7), genes of proliferation (STK15 and MYBL2), and genes associated with positive ER status (ESR1 and BCL2). In essence, high expression of HER2-amplicon and/or proliferation genes gives a high Recurrence Score (and correlates with LumB because most HER2+ and ER+ tumors fall into this subtype), while low expression of these genes and high expression of ER status genes gives a low Recurrence Score (and correlates with LumA).

This data shows that the breast tumor intrinsic subtypes identified using the Intrinsic/UNC gene list can be generalized to many different patient sets, both treated and untreated. The intrinsic portraits of breast tumors are recognizable patterns of expression that are of biological and clinical value, and the SSP-based classification tool represents an unchanging predictor to be used for individualized medicine.

### 3. Example 3

### Agreement in Breast Cancer Classification Between Microarray and qRT-PCR from Fresh-Frozen and Formalin-Fixed Paraffin-Embedded Tissues

Microarray analyses of breast cancers have identified different biological groups that are important for prognosis and treatment. In order to transition these classifications into the clinical laboratory, a real-time quantitative (q)RT-PCR assay has been developed for profiling breast tumors from formalin-fixed paraffin-embedded (FFPE) tissues and evaluate its performance relative to fresh-frozen (FF) RNA samples.

Micro arraydata from 124 breast samples were used as a training set for classifying tumors into four different previously defined molecular subtypes of Luminal, HER2+/ER−, Basal-like, and Normal-like. Sample class predictors were developed from hierarchical clustering of microarray data using two different centroid-based algorithms: Prediction Analysis of Microarray and a Single Sample Predictor. The training set data was applied to predicting sample class on an independent test set of 35 breast tumors procured as both fresh-frozen and formalin-fixed, paraffin embedded tissues (70 samples). Classification of the test set samples was determined from microarray data using a large 1300 gene set, and using a minimized version of this gene list (40 genes). The minimized gene set was also used in a real-time qRT-PCR assay to predict sample subtype from the fresh-frozen and formalin-fixed, paraffin embedded tissues. Agreement between primer set performance on fresh-frozen and formalin-fixed, paraffin embedded tissues was evaluated using diagonal bias, diagonal correlation, diagonal standard deviation, concordance correlation coefficient, and subtype assignment.

The centroid-based algorithms (Prediction Analysis of Microarray and Single Sample Predictor) had complete agreement in classification from formalin-fixed, paraffin-embedded tissues using qRT-PCR and the minimized ‘intrinsic’ gene set (40 classifiers). There was 94% (33/35) concordance between the diagnostic algorithms when comparing subtype classification from fresh-frozen tissue using microarray (large and minimized gene set) and qRT-PCR data. By qRT-PCR alone, there was 97% (34/35) concordance between fresh-frozen and formalin-fixed, paraffin embedded tissues using Prediction Analysis of Microarray and 91% (32/35) concordance using Single Sample Predictor. Finally, we used several analytical techniques to assess primer set performance between fresh-frozen and formalin-fixed, paraffin-embedded tissues and found that the ratio of the diagonal standard deviation to the dynamic range was the best method for assessing agreement on a gene-by-gene basis.

Determining agreement in classification between platforms and procurement methods requires a variety of methods. It has been shown that centroid-based algorithms are robust classifiers for breast cancer subtype assignment across platforms (microarray and qRT-PCR data) and procurement conditions (fresh-frozen and formalin-fixed, paraffin-embedded tissues). In addition, the standard deviation, dynamic range, and concordance correlation coefficient are important parameters to assess individual primer set performance across procurement methods. The strategy for primer set validation and classification have

applications in routine clinical practice for stratifying breast cancers and other tumor types.

Expression-based classifications are important for determining risk of relapse and making treatment decisions in breast cancer (Fan et a. N Engl J Med 2006, 355:560-569; Paik et al. N Engl J Med 2004, 351:2817-2826; Perou et al. Nature 2000, 406:747-752; van't Veer et al. Nature 2002, 415:530-536). Classifications are often developed using microarray data and then further validated on the same or different platforms using minimized gene sets. For instance, van't Veer and van de Vijver used microarray data in training and test sets to validate a 70-gene signature that predicts relapse in early stage ER-positive and ER-negative tumors (van't Veer et al. Nature 2002, 415:530-536; van de Vijver et al. N Engl J Med 2002, 347:1999-2009). In addition, Paik et al developed a 16-gene classifier that predicts relapse in ER-positive tumors using qRT-PCR on formalin-fixed, paraffin embedded (FFPE) tissues. Furthermore, Perou and Sorlie showed that hierarchical clustering of microarray data separates breast tumors into different ‘biological’ subtypes (Luminal, HER2+/ER−, Basal-like, and Normal-like) and that these subtypes are prognostic (Sorlie et al. Proc Natl Acad Sci USA 2001, 98:10869-10874). The biological classification has been validated on multiple patient cohorts using cross-platform microarray analyses and qRT-PCR (Hu et al. BMC Genomics 2006, 7:96; Perreard et al. Breast Cancer Res 2006, 8:R23; Sorlie et al. Proc Natl Acad Sci USA 2003; 100:8418-8423).

Although there are few genes in common between those used to determine the biological subtypes and those used in other classifications for breast cancer prognosis, the different tests identify similar properties that predict tumor behavior (Fan et al. N Engl J Med 2006, 355:560-569). A major difference between the classification for biological subtypes and other classifications for breast cancer is that it is based on hierarchical clustering. The unsupervised nature of hierarchical clustering is effective for discovery (Eisen et al. Proc Natl Acad Sci USA 1998, 95:14863-14868), but it is not suitable for predicting a new sample's class since dendrogram associations can change when new data is introduced. However, it is possible to classify samples within the framework of hierarchical clustering using centroid-based methods (Tibshirani et al. Proc Natl Acad Sci USA 2002, 99:6567-6572; Bair et al. PLoS Biol 2004, 2:E108; Bullinger et al. N Engl J Med 2004, 350:1605-1616). For instance, Tibshirani et al has shown that the nearest shrunken centroid method, used in Prediction Analysis of Microarray (PAM), can classify samples as accurately as statistical approaches like artificial neural networks. In addition, Hu et al employed another simple centroid method called Single Sample Predictor (SSP) to classify subtypes of breast cancer (Hu et al. 2006).

a) Materials and Methods

(1) Tissue Procurement and Processing

All tissues and data used in this study were collected and handled in compliance with federal and institutional guidelines. Breast samples received in pathology were flash frozen in liquid nitrogen and stored at −80° C. Samples were procured at the University of North Carolina at Chapel Hill, Thomas Jefferson University, University of Chicago, and University of Utah. The 159 breast samples analyzed included a 124-sample microarray training set and a 35-sample test set profiled by microarray and real-time qRT-PCR (FF and FFPE). Total RNA from FF samples was isolated using the RNeasy Midi Kit (Qiagen, Valencia, Calif.) and treated on-column with DNase I to eliminate contaminating DNA. The RNA was stored at −80° C. until used for cDNA synthesis.

Each FF sample in the test set was compared to the clinical FFPE tissue block. An H&E slide was used to confirm the presence of >50% tumor and 20 micron cuts were prepared using a microtome. Tissue blocks were 1-5 years in age (i.e. early age FFPE). The FFPE cut was de-paraffinized in Hemo-De (Scientific Safety Solvents) and washed with 100% ethanol. Total RNA was isolated using the High Pure RNA Paraffin Kit (Roche Molecular Biochemicals, Mannheim, Germany). Manufacturer's instructions were followed for RNA extraction except that the reagents were increased 2-fold for the first proteinase K digestion. Samples were treated with TURBO DNA-free (Ambion, #1906) and stored at −80° C. until cDNA synthesis.

(2) First-Strand cDNA Synthesis

cDNA synthesis for each sample was performed in 40 μl total volume reaction containing 600 ng total RNA. Total RNA was first mixed with 2 μl gene specific cocktail containing 55 primers (each anti-sense primer at 1 pmol/μl) and 2 μl 10 nM dNTP Mix (10 mM each dATP, dGTP, dCTP, dTTP at pH7). Reagents were heated at 65° C. for 5 minutes in a PTC-100 Thermal Cycler (MJ Research, Inc.) and briefly centrifuged. The following reagents were added to each tube: 8 μl 5× First-Strand Buffer, 2 μl 0.1M DTT, 2 μl RNase Out (Invitrogen), and 2 μl Superscript DI polymerase (200 units/p. The reaction was thoroughly mixed by pipetting and incubated at 55° C. for 45 minutes followed by 15 minutes at 70° C. for enzyme inactivation. Following cDNA synthesis, samples were purified with the QIAquick PCR Purification Kit (Qiagen, Valencia, Calif.). Samples were adjusted to a final concentration of 1.25 ng/μl cDNA with TE (10 mM Tris-HCl, pH 8.0, 0.1 mM EDTA).

(3) Primer Design and Optimization

Primers were designed using Roche LightCycler Probe Design Software 2.0. Reference gene sequences were obtained through NCBI LocusLink and optimal primer sites were found with the aid of Evidence Viewer (http://www.ncbi.nlm.nih.gov). Primers sets were selected to avoid known insertions/deletions and mismatches while including all isoforms possible. Amplicons were limited to 60-100 bp in length due to the degraded condition of the FFPE mRNA. When possible, RNA specific amplicons were localized between exons spanning large introns (>1 kb). Finally, NCBI BLAST was used to verify gene target specificity of each primer set. Primer sequences are presented in Table 1. Primers were synthesized by Operon, Inc. (Huntsville, Ala.), re-suspended in TE to a final concentration of 60 uM, and stored at −80° C. Each new FFPE primer set was assessed for performance through qRT-PCR runs with three serial 10-fold dilutions of reference cDNA in duplicate and two no template control reactions. Primers were verified for use when they fulfilled the following criteria: 1) target Cp<30 in 10 ng reference cDNA; 2) PCR efficiency >1.75; 3) no primer-dimers in presence of template as determined through post amplification melting curve analysis; and 4) no primer-dimers in negative template control before cycle 40.

(4) Real-Time Quantitative (q)RT-PCR

PCR amplification was carried out on the Roche LightCycler 2.0. Each reaction contained 2 μl cDNA (2.5 ng) and 18 μl of PCR master mix with the following final concentration of reagents: 1 U Platinum Taq, 50 mM Tris-HCl (pH 9.1), 1.6 mM (NH₄)²SO₄, 0.4 mg/μl BSA, 4 mM MgCl₂, 0.2 mM dATP, 0.2 mM dCTP, 0.2 mM dGTP, 0.6 mM dUTP, 1/40000 dilution of SYBR Green I dye (Molecular Probes, Eugene, Oreg., USA), and 0.4 μM of both forward and reverse primers for the selected target. The PCR was done with an initial denaturation step at 94° C. for 90 s and then 50 cycles of denaturation (94° C., 3 s), annealing (58° C., 6 s), and extension (72° C., 6 s). Fluorescence acquisition (530 nm) was taken once each cycle at the end of the extension phase. After PCR, a post-amplification melting curve program was initiated by heating to 94° C. for 15 s, cooling to 58° C. for 15 seconds, and slowly increasing the temperature (0.1° C./s) to 95° C. while continuously measuring fluorescence.

Each PCR run contained a no template control, a calibrator reference in triplicate, and each sample in duplicate. The calibrator reference sample was comprised of 3 breast cancer cell lines (MCF7, SKBR3, and ME16C2) and Stratagene Universal Human Reference RNA (Stratagene, La Jolla, Calif., USA) represented in equal parts. The crossing point (Cₚ) for each reaction was automatically calculated by the Roche LightCycler Software 4.0. Relative quantification was done by importing an external efficiency curve (Eff=1.89) and setting the calibrator at 10 ng for each gene. In order to correct for differences in sample quality and cDNA input, copy numbers were adjusted to the arithmetic mean of 5 ‘housekeeper’ genes (ACTB, PSMC4, PUM1, MRPL19, SF3A1). Values from replicate samples were averaged and data was Iog2 transformed.

(5) Microarray

AU samples were analyzed by DNA microarray (Agilent Human A1, Agilent Human A2, and Agilent custom oligonucleotide microarrays). Labeling and hybridization of RNA for microarray analysis were performed using the Agilent low RNA input linear amplification kit (http://www.chem.agilent.com/Scripts/PDS.asp?lPage:=10003) as described in Hu et al (Hu et al. Biotechniques 2005, 38:121-124). Each sample was assayed versus a common reference that was a mixture of Stratagene's Human Universal Reference total RNA (Stratagene, La Jolla, Calif., USA) enriched with equal amounts of RNA from the MCF7 and ME16C cell lines. Microarray hybridizations were carried out on Agilent Human oligonucleotide microarrays using 2 μg Cy3-labeled ‘reference’ sample and 2 μg Cy5-labeled ‘experimental’ sample.

All microarrays were scanned using an Axon Scanner 4000B (Axon Instruments Inc, Foster City, Calif., USA). The image files were analyzed with GenePix Pro 4.1 (Axon Instruments) and were uploaded into the UNC Microarray Database at the University of North Carolina at Chapel Hill (https://genome.unc.edu/), where a Lowess normalization procedure was performed to adjust the Cy3 and Cy5 channels (Yang et al. Nucleic Acids Res 2002′ 30:e15).

(6) Clinical Immunohistochemistry and PCR

Samples were scored for protein expression at the time of diagnosis using standard operating procedures established at each institution. Greater than 10% positive staining nuclei was considered positive for the ER and PR. Staining and scoring criteria for HER2 were carried out according to the Dako HercepTest™ (Dako, Carpinteria, Calif., USA). Quantitative PCR, used to determine DNA copy number of the ERBB2 gene, was done using a clinical assay from ARUP Laboratories Inc (cat#00049390, Salt Lake City, Utah, USA).

(7) Selecting Genes for Real-Time qRT-PCR

The real-time qRT-PCR assay consisted of 5 housekeeper genes (Szabo et al. Genome Biol 2004, 5:R59), 5 proliferation genes for risk stratification of the Luminal (ER-positive) tumors, and 40 ‘intrinsic’ genes important for distinguishing biological subtypes of breast cancer. The minimal 40 ‘intrinsic’ classifiers were statistically selected from a larger 1300 ‘intrinsic’ gene set previously reported in Hu et al (2006). The larger gene set was minimized as described in Perreard et al (2006). Briefly, a semi-supervised classification method was used in which samples are hierarchical clustered and assigned subtypes based on the sample-associated dendrogram. Samples were designated as Luminal, HER2+/ER−, Basal-like, or Normal-like. The best class distinguishers were identified according to the ratio of between-group to within-group sums of squares. A 10-fold cross-validation was performed using a nearest centroid classifier and testing overlapping gene sets of varying sizes. The smallest gene set which provided the highest class prediction accuracy when compared to the classifications made by the complete microarray-based intrinsic gene set was selected.

(8) Assessing qRT-PCR Agreement Between FF and FFPE Tissues

Thirty-five matched FF and FFPE samples (70 samples total) were analyzed by qRT-PCR using the same primer sets. Agreement in the quantitative data was determined using diagonal bias (m), diagonal spread (d), diagonal standard deviation (dsd), diagonal correlation (r_{d}), and concordance correlation coefficient (CCC).

In diagonal bias, a best fitting line parallel to the diagonal (slope equals 1) is made from a plot of the qRT-PCR data (FF versus FFPE). Numerically, if (x, . . . , y₁), i=1, . . . , n denote the measurement pairs then the best fitting line parallel to the diagonal is given by the expression:

y=x+y̅−x̅

where x and y denote the sample means of the x and y measurements, respectively.

Then diagonal bias is calculated as:

$$m = \frac{\overline{y} - \overline{x}}{\sqrt{2}}$$

The diagonal standard deviation was calculated as follows:

$$\text{?} = \sqrt{\frac{\text{?} {d}_{1}^{2}}{n - 1}}$$

$$\text{?} \text{indicates text missing or illegible when filed}$$

Let d represent:

$$d = \frac{\text{?} {d}_{i}}{n}$$

$$\text{?} \text{indicates text missing or illegible when filed}$$

Diagonal correlation was used to determine the spread of points around the diagonal line:

$${r}_{d} = \frac{2 Cov ( X , Y )}{Var ( X ) + Var ( Y )}$$

This method does not provide information about the extent of deviation but allows measurements with different units to be compared. Further, if we let p denote the correlation coefficient and O_{χ} and O_{γ} the respective standard deviations, then

$${r}_{d} = ρ \frac{2}{\frac{{σ}_{χ}}{{σ}_{γ}} + \frac{{σ}_{γ}}{{σ}_{χ}}}$$

That is, the diagonal correlation penalizes the correction coefficient if there is a scale shift (σₓ≠σ_{y}). The combined effect of the bias and scale shift was measured using the concordance correlation coefficient (CCC) proposed by Lin et al (Lin et al. Biometrics 1989, 45:255-268):

$$C C C = \frac{2 Cov ( X , Y )}{Var ( X ) + Var ( Y ) + {( \overline{Y} - \overline{X} )}^{2}}$$

(9) Assessing Agreement Between Microarray and qRT-PCR for Classification.

A breast cancer subtype predictor was developed in PAM (http://www-stat. Stanford. edu/˜tibs/P AMA and SSP using 124 breast samples and the ‘intrinsic’ gene set identified in Hu et al (2006). The training set contained representative samples of Luminal (64 samples), HER2+/ER− (23 samples), Basal-like (28 samples), and Normal-like (9 samples) subtypes. Classification of an independent test set (35 matched FF and FFPE samples) was done using a large (1300 genes) and minimized (40 genes) version of the ‘intrinsic’ set. Subtypes were assigned based on Spearman correlation to the centroid. The qRT-PCR data from the test set was merged with the microarray data of the training set prior to classification using distance weighted discrimination (Benito et al. Bioinformatics 2004, 20:105-1.14). The gold standard for classification of the training and test samples was based on FF tissue RNA and using the classifications obtained when performing hierarchical clustering analysis using the 1300 gene intrinsic gene set from microarray data,

b) Results

(1) Assessment of qRT-PCR Primer Set Performance by Comparing Agreement Between FF and FFPE Tissues.

The data set of 35 matched FF and FFPE tissues (70 samples) was evaluated for 50 genes using the same PCR conditions. Agreement between FF and FFPE tissues was assessed for diagonal bias (m), diagonal correlation (r_{d}) diagonal standard deviation (dsd), and concordance correlation coefficient (ccc). An agreement plot between FF and FFPE for the estrogen receptor gene (ESR1) was produced after normalization to the 5 housekeepers. The large dynamic range of ESR1 expression provides clear separation of the tumors from both FF and FFPE. ESR1 alone measured from FF tissue has very high sensitivity and specificity using ER status by IHC as the gold standard (Perreard 2006).

For each gene, the agreement between FF and FFPE was analyzed using the raw data, housekeeper normalized data, and DWD adjusted normalized data. Scatter plots are provided in FIGS. 20-23 and values are presented in Table 14. The line graphs in 19 show the effects at each step of data processing. The raw (pre-normalized) data shows a negative bias for all genes likely due to lower RNA quality in the FFPE tissue (FIG. 19A). Much of the bias was corrected by normalization to the ‘housekeeper’ genes and using DWD adjustment. As expected, DWD had a significant effect on bias (m) but did not effect other measurements of agreement (FIG. 19B-D).

The median biases for the un-normalized, housekeeper normalized, and DWD adjusted normalized data were −1.5 (−3.1 to −0.033), 0.58 (−1.1 to 2) and 0.24 (−0.3 to 1.3), respectively. Normalization to the housekeeper genes had a relatively modest effect on the diagonal standard deviation with a change in the median from 1.1 (0.76-2) to 0.81 (0.38-1.8). While most genes had a similar standard deviation (e.g. ESR1) after applying the housekeepers, other genes such as C10orf7 and COX6C had nearly a 3-fold reduction in standard deviation after normalization.

In general, genes with the highest diagonal correlation between FF and FFPE also had the largest dynamic range in expression (e.g., ESR1, TFF3, COX6C, and FBP1). Housekeeper genes and other genes with low variability in expression (IGBP1) had the lowest diagonal correlation since they form more of a cloud than a line around the diagonal. The housekeeper genes all had high agreement in terms of having low variability in expression across samples in the FF and FFPE tissues.

The concordance correlation coefficient (CCC) considers both bias and scale shift when determining agreement. The median concordance correlation coefficient between FF and FFPE for the raw data of the 45 genes (housekeepers excluded) was 0.28. Normalization to housekeepers raised the CCC median to 0.48, and adjusting with DWD brought the median to 0.61. Only 27% of the genes had a CCC value greater than 0.5, whereas 47% of the genes were above that value in the normalized data, and 76% were above that when using DWD adjusted normalized data. A comparison of the CCC value to the ratio of the diagonal standard deviation over the dynamic range identified many of the same primer sets as good (or poor) performers from the FFPE derived samples.

(2) Breast Cancer Subtype Classification of Test Set Using PAM and SSP.

Hierarchical clustering of the 124 sample training set using the “intrinsic” gene set identified in Hu et al shows 4 distinct classes representing Luminal, HER2+/ER−, Basal-like, and Normal-like. Centroid classifiers were developed from the microarray expression data using PAM and SSP (Hu et al. 2006, Tibshirani et al. 2002). Class predictions were made on the test set using microarray (large and minimized ‘intrinsic’ sets) and qRT-PCR data (15). Each individual microarray (large and minimized) and PCR datasets were DWD merged with the training set prior to subtype class prediction.

Agreement in Classification Between Large and Minimized Microarray Gene Sets.

Thirty-three out of 35 (94%) samples classified the same between PAM and SSP when using the large ‘intrinsic’ microarray dataset for classification. In both discrepant cases, IHC data agreed with the PAM classification. There was the same agreement (94%) when performing the analysis with the minimized version of the microarray data. Interestingly, there was one sample that was called HER2+/ER− by both PAM and SSP when using the large microarray dataset, but called Basal-like by both methods when using the minimized microarray dataset. Additional analysis of this sample by quantitative PCR showed no DNA amplification of HER2/ERBB2 amplicon.

Agreement in Classification Between FF and FFPE.

By qRT-PCR, there was 97% (34/35) concordance between FF and FFPE using PAM, and 91% (32/35) concordance using SSP. There was 94% (33/35) concordance between the diagnostic algorithms from FF tissue and complete agreement in classification from FFPE tissue. Since the FFPE samples were obtained from the clinical block, it is likely that there was a higher tumor percentage in those samples than in the matched FF sample, which could affect the agreement. Indeed, 2 out of the 3 discrepancies in classification made by SSP were when the FF tissue sample was called Normal-like (microarray and PCR) and the FFPE sample was called Luminal (PCR). These samples were ER-positive by IHC and likely Luminal. The only discrepancy in PAM was in a sample classified as Normal-like from FF tissue and Luminal from FFPE.

Overall concordance across methods. Overall, PAM diagnosed 33 out of 35 samples (94%) the same across microarray and qRT-PCR, while SSP diagnosed 30 out of 35 samples (86%) the same across platforms and procurement methods. Discrepancies were of several types including Luminal tumors classified as Normal-like, HER2+/ER− tumors classified as Luminal, and Basal-like tumors classified as HER2+/ER−.

c) Discussion

The transition of large-scale microarray experiments into a clinical test requires identifying a minimum set of genes for classification, translating the assay from microarray to qRT-PCR for routine diagnostics, and validating the assay using both FF and FFPE specimen types.

A previous qRT-PCR assay for identifying biological subtypes was based on an intrinsic gene set derived from first generation microarrays that contained 8,100 genes. In comparison, the current intrinsic set was derived from a different microarray platform (cDNA versus Agilent), contained a larger number of genes (427 vs. 1300), and used pre-treatment samples only (Hu et al. 2006. The overlap in the minimized gene set developed here versus the list in Perreard et al. was 14 out of 40, which is not surprising since there were only 108 genes in common between the larger intrinsic gene sets. It has been shown that the new intrinsic gene set reproducibly identifies the same breast cancer subtypes within independent datasets (i.e. pure training and test sets), and that the biological classification adds significant clinical information when used in a multivariate Cox analysis.

It has been shown that the centroid-based method called Single Sample Predictor can use microarray data to classify breast cancers into biological subtypes that predict survival in treated and untreated patients (Hu et al. 2006). Here PAM is directly compared to SSP using the large microarray dataset applied in Hu et al, and also tested a minimized version using microarray and qRT-PCR data. Both methods performed well.

This method of classification is considered semi-supervised since data from hierarchical clustering is initially used to develop a centroid or shrunken centroid from a training set and new samples are then classified based on the distance to the centroid. In this way, the training set is not only necessary for initial discovery and validation but the data continues to be used as a reference base for future classification of new samples. Similarly, the Oncotype Dx assay established cut points for risk of relapse from a training set and this classifier rule is applied to new samples to derive a recurrence score.

Determining agreement between methods is a complex issue that requires consideration of several factors before reaching a conclusion. Cronin et al used Pearson correlation to show that the genes with the highest correlation in microarray maintained their association with qRT-PCR. They used short amplicons and control ‘housekeeper’ genes in the qRT-PCR assay to correct biases between FF and FFPE tissues. Although correlation provides information about the linearity and slope (positive or negative correlation) of the data, it does not indicate the amount of bias, scale shift, or data spread. These additional measurements are helpful in determining whether the discrepancies in the data can be compensated for experimentally (e.g., housekeeper genes) or by software algorithms. For example, when the qRT-PCR data from FF and FFPE were compared, it was found that a significant bias could be corrected by normalization to the housekeepers and applying Distance Weighted

Discrimination. Distance Weighted Discrimination corrected systematic biases but did not change other measurements of agreement. After correcting for systematic bias, it is then possible to evaluate variation due to noise that cannot be predicted or controlled.

It was found that the most useful analyses for assessing PCR primer set performance across FF and FFPE tissues were the concordance correlation coefficient, the diagonal standard deviation, and the dynamic range. Genes with a large dynamic range often had high correlation and were good classifiers across conditions, even with relatively large diagonal standard deviations. Although genes with a small dynamic range can be good classifiers, the measurement may not be as reproducible if there is a large amount of variation. Thus, it was found that the best assessment of a classifier was using a ratio of the diagonal standard deviation to the dynamic range. This allowed genes with smaller dynamic ranges to be considered as good classifiers, if they also had low diagonal standard deviations. The concordance correlation coefficient and the ratio of the diagonal standard deviation to the dynamic range selected many of the same genes as having similar performance from the FF and FFPE tissues.

Translating an assay from microarray to qRT-PCR provides a second level of gene validation and allows the test to be used on archived FFPE tissue blocks from clinical trials or on samples submitted for routine diagnostics (Paik et al. 2004; Cronin et al. Am J Pathol 2004, 164:35-42). qRT-PCR on formalin-fixed paraffin-embedded tissue can be effectively used for gene expression based diagnostics for translation into the clinical laboratory. The FFPE procured RNA provided accurate subtype classifications in breast cancer, and in some instances provided more tumor specific information than the FF derived samples. This study also developed methodologies that have wider application for developing qRT-PCR assays for subtype classification in a wide variety of cancer types. These gene expression based tests can provide powerful new prognostic clinical tools and aid in more appropriate individualized treatment decisions.

TABLE 11
223. Regression model using RFS and the intrinsic classes
from the 315 tumor sample Combined Test Set.

| | Hazard | | | | | Std |
|---|---|---|---|---|---|---|
| | Ratio | p-value | 95% CI lower | 95% CI upper | Param Est | Error |
| age (decade) | 1.079 | 0.2949 | 0.936133111 | 1.242912729 | 0.07573 | 0.07231 |
| ER | 0.692 | 0.1404 | 0.42483297 | 1.128714303 | −0.36749 | 0.24927 |
| Node status | 1.35 | 0.1261 | 0.919128261 | 1.981847717 | 0.29985 | 0.19601 |
| Grade 1 vs. 2 | 1.879 | 0.1376 | 0.817125651 | 4.322363677 | 0.63092 | 0.42494 |
| Grade 1 vs. 3 | 2.576 | 0.0321 | 1.084274609 | 6.120897891 | 0.94631 | 0.44153 |
| size | 1.591 | <0.0001 | 1.300348623 | 1.947657951 | 0.46463 | 0.10306 |
| LumA vs. Basal-like | 2.023 | 0.0358 | 1.047852886 | 3.904839964 | 0.70448 | 0.33558 |
| LumA vs. HER2+/ER− | 3.468 | 0.0003 | 1.780768834 | 6.75548522 | 1.2437 | 0.34013 |
| LumA vs. LumB | 1.923 | 0.0284 | 1.071712675 | 3.449405854 | 0.65373 | 0.2982 |
| LumA vs. LumI | 1.401 | 0.3669 | 0.673503019 | 2.914105175 | 0.33715 | 0.37368 |
| LumA vs. Normal-like | 1.556 | 0.3686 | 0.589038578 | 4.163947739 | 0.4486 | 0.49891 |

TABLE 7
SAMPLES CLINICAL DATA

| | | | ER (1 = positive; | | | | | |
|---|---|---|---|---|---|---|---|---|
| | | | 0 = negative: | | | Size (1 = <=2 cm; | | |
| | | | If fmol = 10 = + | | | 2 = >2 cm | | RFS |
| | | | (used fmol for | | | to <=5 cm; | | event (0 = |
| | | | rosetta and singapore) | | | 3 = >5 cm; | | no relapse, |
| | | | and norway as | | | 4 = any size with | | 1 = relapsed or |
| | | | detailed in PNAS | | | direct extension | | died |
| Sample Name | Age | Race | 2003 Table | HER2 | PGR | to chest wall or skin) | Grade | of disease) |
| 02573-BC-PRIMARY | 41 | AA | 1 | | | 3 | 3 | 1 |
| A1-17-left-breast-T | 84 | C | 0 | 0 | | 4 | 3 | 1 |
| A4-LUL_Lung-Mel | 44 | C | 1 | | | 4 | 3 | 1 |
| A5-Skin#1_Right-Mel | 85 | AA | 0 | | | 4 | 3 | 1 |
| BC00010 | 47 | C | 0 | | | 3 | 2 | 1 |
| BC00014T | 88 | AA | 0 | | | 4 | 3 | 1 |
| BC00024 | 88 | AA | 0 | | | 3 | 3 | 1 |
| BC00020 | 44 | C | 0 | | 0 | | 3 | 0 |
| BC00034 | 88 | AA | 0 | | | 1 | 2 | 0 |
| BC00038 | 55 | AA | 0 | | | 2 | 2 | 0 |
| BC0004 | 87 | C | 0 | | | 1 | 2 | 0 |
| BC00041T | 46 | AA | 0 | 0 | | 2 | 3 | 1 |
| BC00043T | 43 | C | 0 | | | 2 | 3 | 0 |
| BC00048 | 43 | C | 0 | | | 2 | 3 | 1 |
| BC00051 | 51 | C | 0 | | | 2 | 2 | 0 |
| BC00052 post chemo | 47 | AA | 0 | 2 | | 2 | 3 | 1 |
| BC80053 | 71 | AA | 0 | | | 2 | 3 | 1 |
| BC00057 post chemo | 51 | AA | 0 | 3 | | 4 | 3 | 1 |
| BC00064 RECUR | 44 | C | 0 | 2 | | 1 | 3 | 1 |
| BC00068 | 43 | AA | 0 | 3 | | 3 | 3 | 1 |
| BC00070 | 38 | AA | 0 | 0 | | 2 | 2 | 1 |
| BC00071 | 33 | C | 0 | | | 2 | 2 | 1 |
| BC00078 | 88 | AA | 0 | 0 | | 3 | 3 | 0 |
| BC00082 | 84 | AA | 0 | 0 | | 2 | 3 | 0 |
| BC00085 | 24 | AA | 0 | 1 | | 1 | 2 | 0 |
| BR00-0344B | 65 | AA | 1 | 0 | 0 | 2 | 3 | 1 |
| BR00-0365B | 43 | AA | 1 | 2 | 1 | 4 | 3 | 0 |
| BR00-0387B | 57 | AA | 1 | 0 | 1 | 4 | 2 | 1 |
| BR00-0504B | 88 | C | 1 | 0 | 1 | 2 | 1 | 0 |
| BR00-0572B | 45 | AA | 0 | 3 | 0 | 3 | 3 | 1 |
| BR00-0587B | 68 | C | 1 | 2 | 1 | 2 | 3 | 0 |
| BR00-284B | 63 | C | 0 | 3 | 0 | 3 | 3 | 0 |
| BR01-0125B | 40 | C | 1 | 0 | 1 | 3 | 2 | 0 |
| BR01-0246B | 36 | Other | 1 | 0 | 1 | 2 | 2 | 0 |
| BR01-0349B | 37 | C | 1 | 3 | 0 | 3 | 3 | 1 |
| BR94-1083B | 48 | C | 0 | 3 | 1 | 1 | 3 | 1 |
| BR95-0035B | 74 | C | 0 | 3 | 0 | 2 | 3 | 0 |
| BR05-0152B | 72 | C | 1 | 3 | 0 | 4 | 3 | 1 |
| BR95-0184B | 74 | C | 1 | 3 | 0 | 2 | 3 | 0 |
| BR96-0014B | 47 | AA | 1 | 3 | 1 | 4 | 1 | 0 |
| BR97-0137B | 55 | Other | 0 | | 0 | 3 | 3 | 1 |
| BR98-0181B | 57 | AA | 0 | 3 | 0 | 2 | 2 | 1 |
| BR98-0281B | 44 | C | 0 | 3 | 0 | 2 | 3 | 0 |
| BR99-0207B | 84 | C | 1 | 0 | 0 | 2 | 2 | 0 |
| BR99-0348B | 85 | AA | 1 | 2 | 1 | 2 | 2 | 0 |
| HCl00-039 | | | | | | | | |
| HCl00-052 | | | | | | | | |
| HCl00-088L | | | | | | | | |
| HCl00-192 | | | | | | | | |
| HCl00-283 | | | | | | | | |
| HCl01-041 | | | | | | | | |
| HCl01-155 | | | | | | | | |
| HCl02-235 | 57 | C | 0 | 0 | 0 | 2 | 3 | |
| HCl02-264 | 50 | C | 1 | 1 | 1 | 3 | 0 | 1 |
| MB75 | 53 | | 1 | 3 | 1 | 2 | 3 | 0 |
| MB76 | 57 | | 1 | 0 | 1 | 2 | 2 | 0 |
| MB77 | 63 | | 1 | 0 | 1 | 2 | 3 | 0 |
| MB70 | 50 | | 0 | 2 | 0 | 2 | 3 | |
| MB79 | 63 | | 1 | 3 | 1 | 2 | 3 | 0 |
| MB80 | 50 | | 1 | 0 | 1 | 2 | 3 | |
| MB81 | 84 | | 1 | 0 | 1 | 2 | 2 | 1 |
| MB83 | 31 | | 0 | 0 | 0 | 2 | 3 | |
| MB85 | 77 | | 1 | 0 | 1 | 2 | 3 | |
| MB86(LN) | 72 | | 0 | 0 | 0 | | 3 | 1 |
| MB87 | 73 | | 0 | 0 | 0 | | 3 | |
| PB120-MET-L | 61 | AA | 0 | | | 2 | 3 | 1 |
| PB126 | 29 | AA | 0 | 0 | 0 | 4 | 3 | 1 |
| PB126-MET-LN | | AA | 0 | 0 | 0 | 4 | 3 | 1 |
| PB138 | 58 | C | 0 | 0 | 0 | 2 | 2 | 0 |
| PB140 | 41 | C | 1 | 2 | 1 | 2 | 1 | 0 |
| PB152-MET-LN | | C | 0 | 1 | 0 | | | |
| PB158T | 86 | AA | 1 | | | 3 | 3 | 0 |
| PB184 | 50 | C | 1 | 3 | 0 | 1 | 3 | 0 |
| PB205T | 39 | C | 0 | 1 | 0 | 4 | 2 | 0 |
| PB244 | 38 | AA | 0 | 3 | 0 | 1 | 3 | 0 |
| PB249 | 36 | C | 1 | 3 | 1 | 1 | 3 | 0 |
| PB256 | 58 | C | 1 | 2 | 1 | 2 | 3 | 0 |
| PB257 | 44 | AA | 0 | 2 | 0 | 2 | 3 | 0 |
| PB271 | 45 | AA | 1 | 3 | 1 | 2 | 3 | 0 |
| PB277 | 48 | C | 1 | 2 | 1 | 2 | 3 | 0 |
| PB284 | 34 | C | 1 | 2 | 1 | 1 | 1 | |
| PB293 | 56 | C | 1 | 2 | 1 | 2 | 2 | 0 |
| PB297 | 55 | AA | 0 | 1 | 0 | 2 | 3 | 0 |
| PB307 | 35 | | 1 | 1 | 1 | 3 | 3 | 0 |
| PB311 | 48 | C | 1 | 0 | 1 | 3 | 3 | 0 |
| PB314 | 51 | C | 0 | 3 | 0 | 3 | 3 | 0 |
| PB334 | 50 | AA | 0 | 0 | 0 | 1 | 3 | 0 |
| PB370 | 67 | C | 1 | 0 | 1 | 2 | 3 | 0 |
| PB376 | 50 | AA | 0 | 1 | 0 | 2 | 3 | 0 |
| PB377 | 77 | C | 1 | 1 | 0 | 2 | 3 | 0 |
| PB378 | 55 | Other | 1 | 1 | 1 | 2 | 3 | 0 |
| PB388 | 80 | C | 1 | 1 | 1 | 2 | 3 | 0 |
| PB407 | 56 | C | 1 | 0 | 1 | 3 | 3 | |
| PB413 | 63 | AA | 1 | 0 | 1 | 2 | 2 | 0 |
| PB419 | 49 | C | 0 | 0 | 0 | 2 | 3 | 0 |
| PB432 | 79 | | 1 | 1 | 1 | 2 | 3 | |
| PB441 | 83 | C | 1 | 0 | 1 | 1 | 2 | 0 |
| PB455 | 52 | AA | 0 | 3 | 0 | 3 | 2 | 0 |
| PB475 | 50 | C | 1 | 0 | 1 | 2 | 2 | 0 |
| PB479 | 52 | Asian | 1 | 0 | 1 | 2 | 3 | |
| PB516 | | AA | 0 | 0 | 0 | 3 | 3 | |
| UB21 | 77 | | 1 | 0 | 1 | 1 | 1 | 0 |
| UB22 | | | | | | | | 0 |
| UB27 | 91 | C | 1 | 2 | 1 | 3 | 2 | 0 |
| UB28 | 46 | C | 0 | 0 | 0 | 1 | 3 | 0 |
| UB28A | 59 | C | 0 | 0 | 0 | 2 | 3 | 0 |
| UB37 | 42 | C | 0 | 2 | 1 | 1 | 3 | 0 |
| UB38 | 50 | C | 1 | 0 | 1 | 1 | 1 | 0 |
| UB39 | 48 | C | 1 | 0 | 0 | 1 | 2 | 0 |
| UB43 | 48 | C | 1 | 1 | 1 | 1 | 3 | 0 |
| UB44 | 50 | C | 1 | 0 | 1 | 2 | 3 | 0 |
| UB45 | 46 | C | 1 | 1 | 1 | 2 | 2 | 0 |
| UB55 | 58 | C | 1 | 2 | 1 | 1 | 1 | 0 |
| UB57 | 60 | C | 1 | 0 | 1 | 1 | 2 | 0 |
| UB58 | 58 | C | 1 | 1 | 1 | 1 | 4 | 0 |
| UB60 | 72 | C | 0 | 3 | 0 | 2 | 3 | 0 |
| UB61 | 51 | C | 1 | 3 | 0 | 2 | 2 | 0 |
| UB62 | 28 | C | | | | 1 | 1 | 0 |
| UB64 | 87 | C | 1 | 3 | 1 | 2 | 2 | 0 |
| UB66 | 88 | Other | 1 | 0 | 1 | 2 | 1 | 0 |
| UB67 | 90 | C | 0 | 0 | 0 | 1 | 3 | 0 |
| UB69 | 40 | C | 1 | 0 | 8 | 1 | 1 | 0 |
| UB78 | 41 | hlsp | 1 | 0 | 0 | 4 | 2 | 1 |
| UB79 | 48 | | 1 | 1 | 0 | 2 | 2 | 0 |

| | | | number | Overall | | |
|---|---|---|---|---|---|---|
| | | | of | Survival | | |
| | | number | nodes | Event | | |
| | | of | positive | (0 = alive, | Overall | |
| | RFS | nodes | for | 1 = DOD | suvival | |
| Sample Name | months | examined | tumor | or DOC) | months | Important Comments |
| 02573-BC-PRIMARY | 10 | 25 | 14 | 0 | 22 | primary for a patient with an associated brain |
| A1-17-left-breast-T | 2 | | | 1 | 2 | Autopsy Patient Sample |
| A4-LUL_Lung-Mel | 22 | | | 1 | 22 | Autopsy Patient Sample |
| A5-Skin#1_Right-Mel | 28 | 44 | 3 | 1 | 28 | Autopsy Patient Sample |
| BC00010 | 18 | 21 | 19 | 1 | 16 | |
| BC00014T | 18 | 40 | 38 | 1 | 23 | |
| BC00024 | 3 | 116 | 14 | 1 | 3 | pt was diagnosed with MM at same time as [symbol] |
| BC00020 | 82 | 7 | 3 | 0 | 62 | lymph node met sample? - no primary tumor [symbol] |
| BC00034 | 81 | | | 0 | 81 | |
| BC00038 | 10 | 23 | 1 | 0 | 10 | |
| BC0004 | 118 | 20 | 0 | 0 | 118 | |
| BC00041T | 13 | 19 | 0 | 1 | 29 | |
| BC00043T | 78 | 24 | 0 | 0 | 76 | |
| BC00048 | 48 | 13 | 1 | 0 | 72 | her2 was 1+ on recurrent tumor, not done on [symbol] |
| BC00051 | 88 | 12 | 12 | 0 | 68 | |
| BC00052 post chemo | 14 | 13 | 9 | 1 | 18 | pt had LABC, had neoadj chemo, this specim [symbol] |
| BC00053 | 27 | 21 | 7 | 1 | 28 | |
| BC00057 post chemo | 8 | 9 | 9 | 1 | 12 | pt had IBC, had neoadj chemo, this specimen |
| BC00064 RECUR | 10 | | | 1 | 47 | pt had local recurrence (this is the sample w [symbol] |
| BC00068 | 18 | 38 | 4 | 1 | 18 | |
| BC00070 | 22 | | | 1 | 25 | contralateral breast cancer dx Nov. 15, 2000, dx w [symbol] |
| BC00071 | 16 | 20 | 4 | 1 | 47 | |
| BC00078 | 12 | 16 | 12 | 1 | 12 | cirrhosis was cause of death |
| BC00082 | 27 | 3 | 0 | 1 | 27 | pt admitted with CHF/NQWMI, prob died of a [symbol] |
| BC00085 | 18 | | | 0 | 19 | extensive DCIS w/multiple.small foci of invas [symbol] |
| BR00-0344B | 7 | 15 | 2 | 1 | 30 | |
| BR00-0365B | 22 | 8 | 6 | 0 | 22 | |
| BR00-0387B | 8 | 17 | 10 | 0 | 51 | |
| BR00-0504B | 39 | 15 | 1 | 0 | 39 | |
| BR00-0572B | 11 | 31 | 7 | 0 | 42 | |
| BR00-0587B | 37 | 14 | 0 | 0 | 37 | |
| BR00-284B | 43 | 6 | 0 | 0 | 43 | |
| BR01-0125B | 33 | 17 | 1 | 0 | 33 | |
| BR01-0246B | 31 | 15 | 9 | 0 | 31 | |
| BR01-0349B | 3 | 24 | 22 | 1 | 24 | |
| BR94-1083B | 23 | 19 | 1 | 1 | 47 | |
| BR95-0035B | 106 | 13 | 1 | 0 | 106 | |
| BR05-0152B | 26 | 15 | 0 | 0 | 101 | |
| BR95-0184B | 96 | 20 | 1 | 0 | 96 | |
| BR96-0014B | 86 | | | 0 | 96 | |
| BR97-0137B | 20 | 21 | 1 | 1 | 21 | died of Unconfirmed mel ca (symptoms of me [symbol] |
| BR98-0181B | 36 | 24 | 0 | 1 | 80 | |
| BR98-0281B | 55 | 14 | 0 | 0 | 65 | |
| BR99-0207B | 57 | 5 | 1 | 0 | 57 | |
| BR99-0348B | 32 | 33 | 0 | 0 | 32 | died of other causes (daydration secondary to |
| HCl00-039 | | | | | | |
| HCl00-052 | | | | | | |
| HCl00-088L | | | | | | |
| HCl00-192 | | | | | | |
| HCl00-283 | | | | | | |
| HCl01-041 | | | | | | |
| HCl01-155 | | | | | | |
| HCl02-235 | | 12 | 0 | | | |
| HCl02-264 | 0 | 20 | 0 | | | ER positive tumor (5 cml) but no positive node |
| MB75 | 20 | 15 | 0 | 0 | 20 | |
| MB76 | 22 | 11 | 0 | 0 | 22 | |
| MB77 | 22 | 17 | 0 | 0 | 22 | Had right breast radical mastectomy in 1979 [symbol] |
| MB70 | | 5 | 4 | | | |
| MB79 | 19 | 7 | 0 | 0 | 18 | |
| MB80 | | 0 | 0 | | | |
| MB81 | 13 | 1 | 1 | 0 | 16 | Several recurences (cutaneous, gastric) |
| MB83 | | 17 | 0 | | | |
| MB85 | | 11 | 2 | | | |
| MB86(LN) | 15 | 17 | 7 | 0 | 41 | Lymph node metastasis - Saveral recurrences |
| MB87 | | 1 | 1 | | | metastasis in small intestine |
| PB120-MET-L | 1 | 15 | 14 | 1 | 13 | lymph node metastasis sample this patient w [symbol] |
| PB126 | 1 | 7 | 7 | 1 | 16 | This patient was never disease-free and died |
| PB126-MET-LN | 1 | 7 | 7 | 1 | 18 | |
| PB138 | 30 | 0 | 0 | 0 | 90 | |
| PB140 | 34 | 10 | 0 | 0 | 34 | |
| PB152-MET-LN | | | | | | ER, Her2 and PGR sts for PB152 but maybe |
| PB158T | 30 | 0 | 0 | 0 | 30 | |
| PB184 | 29 | 2 | 0 | 0 | 29 | |
| PB205T | 5 | 7 | 1 | 0 | 5 | |
| PB244 | 24 | 12 | 0 | 0 | 24 | |
| PB249 | 8 | 3 | 3 | 0 | 8 | |
| PB256 | 4 | 14 | 1 | 0 | 4 | |
| PB257 | 20 | 32 | 1 | 0 | 20 | |
| PB271 | 14 | 12 | 3 | 0 | 14 | |
| PB277 | 12 | 18 | 9 | 0 | 12 | |
| PB284 | | 0 | 0 | | | |
| PB293 | 11 | 12 | 0 | 0 | 11 | |
| PB297 | 18 | 0 | 0 | 0 | 18 | |
| PB307 | 9 | 15 | 0 | 0 | 9 | |
| PB311 | 14 | 12 | 2 | 0 | 14 | |
| PB314 | 21 | 13 | 8 | 0 | 21 | |
| PB334 | 19 | 0 | 0 | 0 | 19 | |
| PB370 | 20 | 11 | 2 | 0 | 20 | |
| PB376 | 15 | 3 | 0 | 0 | 15 | |
| PB377 | 18 | 8 | 0 | 0 | 18 | there are 2 different tumors within the same b [symbol] |
| PB378 | 17 | 12 | 4 | 0 | 17 | |
| PB388 | 18 | 5 | 0 | 0 | 15 | |
| PB407 | | 11 | 6 | | | |
| PB413 | 9 | 9 | 3 | 0 | 9 | |
| PB419 | 10 | 1 | 0 | 0 | 10 | |
| PB432 | | 21 | 4 | | | |
| PB441 | 8 | 0 | 0 | 0 | 9 | bilateral breast cancer and renal carcinoma |
| PB455 | 8 | 8 | 3 | 0 | 9 | |
| PB475 | 2 | 5 | 0 | 0 | 2 | |
| PB479 | | 19 | 1 | | | |
| PB516 | | 14 | 2 | | | IDS and DCIS |
| UB21 | 30 | 1 | 0 | 0 | 30 | |
| UB22 | 25 | | | | 25 | no malig (fibroadenoma) |
| UB27 | 29 | 14 | 2 | 0 | 29 | |
| UB28 | 30 | 20 | 0 | 0 | 30 | |
| UB28A | 25 | 19 | 0 | 0 | 25 | |
| UB37 | 25 | 14 | 3 | 0 | 25 | |
| UB38 | 20 | 13 | 0 | 0 | 20 | |
| UB39 | 25 | 10 | 0 | 0 | 25 | |
| UB43 | 18 | 14 | 14 | 0 | 19 | |
| UB44 | 24 | 3 | 1 | 0 | 24 | Had the other breast removed (contained mic [symbol] |
| UB45 | 21 | 5 | 1 | 0 | 21 | Had a second small tumor (5 mm - grade 1 - H |
| UB55 | 22 | 4 | 0 | 0 | 22 | |
| UB57 | 17 | 2 | 0 | 0 | 17 | |
| UB58 | 19 | 4 | 1 | 0 | 19 | Graded 1 on the tissue we received (then got |
| UB60 | 20 | 13 | 10 | 0 | 20 | |
| UB61 | 10 | 15 | 9 | 0 | 19 | |
| UB62 | 9 | 23 | 1 | 0 | 9 | No evidence of malignancy (we had IHC valu [symbol] |
| UB64 | 7 | 15 | 0 | 0 | 7 | No follow-up visit (person out of states) |
| UB66 | 9 | 18 | 0 | 0 | 9 | (From Price, utah-chest-X-ray visit used as La [symbol] |
| UB67 | 16 | 15 | 1 | 0 | 10 | |
| UB69 | 13 | 3 | 0 | 0 | 13 | (Can't find IHC data in the database to confirm |
| UB78 | 0 | 20 | 20 | 0 | 14 | has bone metastasis, in abdomen and pelvis - |
| UB79 | 2 | 9 | 2 | 0 | 2 | Macro-metastasis in the lymphnodes - Not in |

[symbol]

indicates data missing or illegible when filed

TABLE 8
Primer Sets and Gene ID

| | | Gene | | |
|---|---|---|---|---|
| | | ID | | |
| Gene symbol | Gene name | (NCBI) | Forward primer | Reverse primer |
| [symbol] | | | | |
| ACADSB | Acyl-Coenzyme A [symbol] | 86 | CTA ACA TAC AAT GCT GCT AGG C | CAA TCT TTG CAT CTC GGA AGT |
| B3GNTS | [symbol] | 84002 | AGA ACT AGG TGG TGT CTA C | GAT TTT CCC TAA CAG GTG C |
| DF | B factor, | 829 | CAT GTG TTC AAA GTC AAG GAT A | TGC TTG TGG TAA TCG GT |
| | proportion | | | |
| C5ORF18 | chromosome 5 [symbol] | 7905 | GTG TTC GGT TAT GGA GC | GGT ATC ATC TTC TTT GTT GGG A |
| (=DP1) | | | | |
| CDK2API | CDK2-associated | 8008 | CGC AGG GAG CAA GAG T | CTT CAA AAC CAA CAA GGC AG |
| | protein 1 | | | |
| CDXDX | cytochrome c | 1345 | AGC TTT GTA TAA GTT TCG TGT | CCA GCC TTC CTC ATC TC |
| | oxidise subunit | | | |
| | Vic | | | |
| CX3CL1 | [symbol] | 6375 | ATG ACA TCA AAG ATA CCT GTA G | GAC CCA TTG CTC CTT CG |
| CYB5 | cytochrome b-5 | 1528 | GCA CCA CAA GGT GTA CG | GCC CGA CAT CCT CAA AG |
| DSC2 (ESTa) | [symbol] | 1024 | GAA TGT GGA GAC TGA AAG CAA | CAA ATG GAG GAT CAT TCT GAT AGG |
| EGFR | Epidermal growth | 1056 | AGG ACA GCA TAG ACG ACA C | AGG ATT CTG CAC AGA GCC A |
| | factor receptor | | | |
| | [symbol] (v-arb-b) | | | |
| | oncogene homolog, | | | |
| | avian) | | | |
| ERBB2 | [symbol] | 2054 | TCC TGT GTG GAC CTG GAT | TGC CGT CGC TTG ATG AG |
| GSR1 | [symbol] receptor 1 | 2009 | CAT GAT CAG GTC CAC CTC TCT | AGC AGC ATG TCG AAG ATC TC |
| FLJ14525 | [symbol] protein | 04005 | CCC TTT CTC CTG GGA AAC | GCT TTG GAC AGT GGT CT |
| | FLJ14525 | | | |
| FOXA1 | Forkhand box A1 | 3160 | GTTAGGAACTGTGAAGATGG | GCCGCTCGTAGTCATG |
| FZD7 | Frizzled homolog 7 | 0324 | AGC CAT TTT GTC CTG TTT TC | CCT TCC TCT TCG TTC ACT |
| | (Drosophilia) | | | |
| GARS | Glycyl-tRNA | 2617 | ACC GAC CGT GAC TCA A | AAA CAG AGG ATA CCT GGC |
| | synthetase | | | |
| GATA3 | GATA binding | 2825 | AAC TGT CAG ACC ACC ACA A | GAA GTC CTC CAG TGA GTC AT |
| | protein 3 | | | |
| GRB7 | Growth factor | 2086 | TCG ATG CAC ACA CTG GTA T | TTC AGA TCT GCC ACG TAC T |
| | receptor-bound | | | |
| | protein 7 | | | |
| G5TP4 | [symbol] | 2050 | GGG CTC TAT GGG AAG G | GTT CTG GGA CAG CAG G |
| HSD1784 | hydroxysteroid | 3296 | TGG GGC TAA GTG GAC TAT | TGC CTT CTG AGG GTC AA |
| | (17-beta) | | | |
| | dehydrogenase 4 | | | |
| KIAA0310 | KIAA0310 [symbol] | 9910 | GCC CTT CTA CAA CCC TG | GCT CCA AGT GCA AGT TC |
| | product | | | |
| KIT | [symbol] | 3815 | CAC GCA CCT GCT GAA AT | TCT ACC ACG GGC TTC TGT C |
| KRT17 | Keratin 17 | 3872 | GAG ATT GCC ACC TAC CG | GAG GAG ATG ACC TTG CC |
| KRT5 | Keratin 5 | 3052 | GGA GAA GGA GTT GGA CC | CCA CTG CTG CTG GAG TA |
| | (epidermolysis [symbol] | | | |
| | simplex, [symbol] | | | |
| NAT1 | N- | 0 | ACA GCA CTC CAG CCA AA | CTG GTA TGA GCG TCC AAA C |
| | acetyltransferase | | | |
| | 1 (arylamine N- | | | |
| | acetyltranferase) | | | |
| PGR | Progeataron a | 5241 | AGC TCA CAG CGT TTC TAT C | TGT GCA GCA ATA ACT TCA GAC |
| | receptor | | | |
| PLGO1 | [symbol] | 5351 | CGT GCC GAC TAT TGA CAT | GTA GCG GAC GAC AAA GG |
| PTP4A2 | proteintyrosino | 6073 | TCA AAG ATT CCA ACG GTC ATA G | TCT CAA GTT CCA CTT CCA GTA G |
| | phosphatase type | | | |
| | IVA, member 2 | | | |
| RABEP1 | Robapitin-5 | 9135 | ATG TCA GTG AGC AAG TCC | GCT GGT TAA TGT CTG TCA GT |
| RARRES3 | [symbol] acid receptor | 6920 | GCT GAG ATA TGG CAA GTC C | CTC CTA ATC GCA AAA GAG C |
| | responder ( [symbol] | | | |
| | induced) 3 | | | |
| S100A11 | S100 calcium | 5282 | CAA AAA TCT CCA GCC CTA CA | TAA CCA TCC TTT CCA GCA TAC |
| | binding protein AB | | | |
| | (calpranulin A) | | | |
| SOC2 | Syndecan 2 | 8359 | AAA CCA CGA CGC TGA AT | ATT TGT ATC CTC TTC GGC TG |
| | (heparan sulfate | | | |
| | protcoglycan 1, | | | |
| | cell surface- | | | |
| | associated, | | | |
| | fibroglycan) | | | |
| SLC39A8 | solute carrier | 25800 | ACC ACC ATA GTC ATA GCC | CAT ACT TGG ACA ACT GCT TC |
| | family 39 (zinc | | | |
| | transporter), | | | |
| | member 2 | | | |
| SLC7A8 | solute carrier | 8057 | AGC GTT TTA CAC CTA TCC C | CCA CGA AGA ACC AGT AGC |
| | family 7 ( [symbol] | | | |
| | amino acid | | | |
| | transporter, y+ | | | |
| | system), member 6 | | | |
| SLPI | secretary | 6590 | GTG TGG GAA ATC CTG CG | GTG GTG GAG CCA AGT CT |
| | leukocyte prolease | | | |
| | inhibitor [symbol] | | | |
| SMA3 | SMA3 | 10571 | CCG TAC CTG ATG CAC GAA | GTG CCC GTA GTT GCG ATA |
| TAP1 | transporter 1, | 8890 | AAG CAC CTC AAC CAG AAG G | GGT AGA GAA CAA ATG TGA CAA GG |
| | ATP-binding [symbol] , | | | |
| | sub-family 0 [symbol] | | | |
| TRIM29 | Triparilla multi- | 23650 | AAC AAC TAC ACG AAC AGC | ATT CTT CTG GGT GGT CTC |
| | combining 29 | | | |
| XBP1 | X-box binding | 7494 | CTG TTG GGC ATT CTG GAC | GGA GGC TGG TAA GGA ACT |
| | protein 1 | | | |
| Pro- | | | | |
| lifiration | | | | |
| genes | | | | |
| BIRC5 | [symbol] IAP repeat- | 332 | CGA CCC CAT AGA GGA ACA TAA | TTC TTG ACA GAA AGG AAA GCG |
| | containing 5 | | | |
| | (yeast) | | | |
| BUB1 | budding | 609 | CAC TTG GGA CTG TTG ATG | TGG ATA GGA ACT CAC TGG T |
| | uninhibited by [symbol] | | | |
| | 1 homolog (yeast) | | | |
| CENPF | Cantromace protein | 1853 | CCA CTG AGT CTC GGC AA | ATT TCG TGG TGG GTT CT |
| | F, 350/400 ka [symbol] | | | |
| CKS2 | CDC protein in | 1164 | TGG AGG AGA CTT GGT GT | GAA TAT GTG GTT CTG GCT CA |
| | kinase regulatory | | | |
| | subunit 2 | | | |
| FAM54A | family with | 113110 | GTG GAA ATG CAG GAA CTG AA | GCT CGT CAC TCA AGC CAA |
| (=DUPD1) | sequence | | | |
| | similarity 56 | | | |
| | member A | | | |
| GTPBP4 | GTP binding | 23560 | GGT GTT GAC ATG GAC GAT AA | CTT CCC GCT TTC TTT TCC TA |
| | protein 4 | | | |
| HSPA14 | heat shock 70 kDa | 61102 | GTT TAG AAG CAA TCA GAG GAC T | CCT CCA CAA AGG ACA ACC |
| | protein 14 | | | |
| MK157 | Antigene | 4250 | TCA GAC TCC ATG TGC CT | CTT CAC TGT CCC TAT GAC TTC |
| | identified by | | | |
| | monoclonal | | | |
| | antibody K1-B7 | | | |
| MYBL2 | v-myb [symbol] viral | 4805 | CAC ACT GCC CAA GTC TCT A | AAG CTG TTG TCT TCT TTG ATA CC |
| | oncogene homolog | | | |
| | [symbol] -like 2 | | | |
| NEK2 | NIMA [symbol] kinas 2 | 4751 | AGC TTG GAG ACT TTG GG | GTA ATA AGG TGT GCC AAC AAA T |
| PCNA | [symbol] cell nuclear | 5111 | GTC ACA GAC AAG TAA TGT CG | TAC TGA GTG TCA CCG TT |
| | [symbol] | | | |
| STK0 | serine/ [symbol] | 6700 | CTT ACT GTC ATT CGA AGA GAG TT | |
| | kinase 6 | | | |
| TDP2A | Topalsomerase | 7153 | AAG CAC ATC AGG TGA AAA AT | TAC CAC AGC CAA TGG CA |
| | (DNA) II alpha | | | |
| | 170 kDa | | | |
| TTK | TTK protein kinase | 7272 | ACG GAA TCA AGT CTT CTA GC | TGC CAC TGT TTC TGG TTA C |
| House- | | | | |
| keeper | | | | |
| genes | | | | |
| MRPL19 | microchondrial | 9601 | GGG ATT TGC ATT CAG AGA TCA G | GGA AGG GCA TCT CGT AAG |
| | ribosimal protein | | | |
| | L1B | | | |
| PEMC4 | [symbol] | 5704 | GGC ATG GAC ATC CAG AAG | CCA CGA CCC GGA TGA AT |
| PLIM1 | Pumilla homolog 1 | 8600 | TGAGGTGTGCACCATGAAC | CAGAATGTGCTTGCCATAGG |
| | [symbol] | | | |

[symbol]

indicates data missing or illegible when filed

TABLE 9
45 Paired Samples for Intrinsic Analysis from Sorlie et al. 2003

shaz111.BC.FUMI05.AF
shaz110.BC.FUMI05.BE
shaz105.BC.FUMI06.AF
shaz104.BC.FUMI06.BE
shaz117.BC.FUMI07.AF
shaz116.BC.FUMI07.BE
shby032.BC.FUMI20.AF
shby020.BC.FUMI20.BE
shaz123.BC.FUMI27.AF
shaz122.BC.FUMI27.BE
shaz115.BC.FUMI35.AF
shaz114.BC.FUMI35.BE
shaz127.BC.FUMI37.AF
shaz126.BC.FUMI37.BE
svl012..BC104A.BE
svl013..BC104B.AF
svl005..BC106A.AF
svl006..BC106B.BE
svcc63..BC107A.AF
svcc98..BC107B.BE
svl003..BC108A.BE
svl004..BC108B.AF
svcc77..BC110A.AF
svcc78..BC110B.BE
svcc97..BC112A.AF
svcc53..BC112B.BE
svcc81..BC114A.BE
svcc52..BC114B.AF
svcc64..BC115A.AF
svcc106.BC115B.BE
svcc112.BC118A.AF
svcc134.BC118B.BE
svl015..BC119A.BE
svl014..BC119B.AF
svl027..BC120A.BE
svl02B..BC120B.AF
svl017..BC121A.AF
svl016..BC121B.BE
svcc91..BC123A.AF
svcc89..BC123B.BE
svcc111.BC124A.BE
svcc109.BC124B.AF
svl018..BC125A.BE
svl019..BC125B.AF
svcc96..BC2
svcc113.BC2.LN2
svcc93..BC206A.BE
svcc135.BC206B.AF
svcc107.BC208A.BE
svcc125.BC208B.AF
svcc79..BC213A.AF
svcc76..BC213B.BE
svcc103.BC214A.AF
svcc92..BC214B.BE
svl021..BC303A.AF
svl020..BC303B.BE
svcc131.BC305A.BE
svcc58..BC305B.AF
svl032..BC307A.AF
svl103..BC307B.BE
svcc115.BC38
svcc116.BC38.LN38
svcc66..BC402B.AF
svcc83..BC402B.BE
svcc36..BC404A.AF
svl033..BC404B.BE
svl029..BC405A.BE
svl030..BC405B.AF
shby035.BC601A.BE
shby036.BC601B.AF
svl042.BC608A.AF
svl036..BC608B.BE
svl040..BC702A.AF
svl041..BC702B.BE
shby034.BC703A.AF
shby037.BC703B.BE
svl039..BC706A.BE
svl038..BC706B.AF
svcc86..BC708A.AF
svcc104.BC708B.BE
svcc85..BC709A.AF
svcc84..BC709B.BE
svcc101.BC710A.BE
svcc82..BC710B.AF
svcc65..BC711A.AF
svcc120.BC711B.BE
svcc105.BC805A.BE
svcc121.BC805B.AF
svcc126.BC808A.AF
svcc124.BC808A.BE

TABLE 10

| Gene | OS~Gene | OS~Gene + Grade | OS~Gene + Stage | OS~Gene + Grade + Stage | Prolif. Gene |
|---|---|---|---|---|---|
| SMA3 | 0.0010086 | 0.00814571 | 0.000398174 | 0.00357674 | NO |
| KIT | 0.000332738 | 0.00154407 | 0.00272027 | 0.00672142 | NO |
| GTPBP4 | 0.00445804 | 0.0307721 | 0.00150072 | 0.0112402 | YES |
| COX6C | 0.00289023 | 0.00951953 | 0.0028745 | 0.0125619 | NO |
| CX3CL1 | 0.00217324 | 0.00425494 | 0.0181299 | 0.0152864 | NO |
| KRT17 | 0.0321012 | 0.0420179 | 0.0233713 | 0.015837 | NO |
| B3GNT5 | 0.032762 | 0.117857 | 0.00427977 | 0.02214 | NO |
| PLOD | 0.00730183 | 0.0152132 | 0.052899 | 0.0406316 | NO |
| SLPI | 0.0533249 | 0.0795638 | 0.0372877 | 0.0608959 | NO |
| DSC2 | 0.0432628 | 0.19777 | 0.0199733 | 0.0720347 | NO |
| GRB7 | 0.0023925 | 0.00997476 | 0.0212037 | 0.076893 | NO |
| TRIM29 | 0.0758398 | 0.0969003 | 0.10943 | 0.0808424 | NO |
| STK6 | 0.0353601 | 0.192395 | 0.0169665 | 0.0990307 | YES |
| BUB1 | 0.0572953 | 0.237675 | 0.0218123 | 0.123044 | YES |
| NAT1 | 0.0127223 | 0.0791954 | 0.0189787 | 0.135405 | NO |
| CYB5 | 0.0557461 | 0.287241 | 0.0273843 | 0.137872 | NO |
| PTP4A2 | 0.160424 | 0.0858591 | 0.342854 | 0.138471 | NO |
| TTK | 0.110921 | 0.45438 | 0.0192107 | 0.143497 | YES |
| HSPA14 | 0.391113 | 0.8142 | 0.0511814 | 0.144083 | YES |
| GATA3 | 0.0324598 | 0.289619 | 0.0175668 | 0.157456 | NO |
| ESR1 | 0.030409 | 0.145509 | 0.0405537 | 0.184542 | NO |
| SLC39A6 | 0.0733459 | 0.430962 | 0.024724 | 0.207555 | NO |
| ERBB2 | 0.0459011 | 0.0828308 | 0.169867 | 0.24427 | NO |
| FOXA1 | 0.110671 | 0.4427 | 0.094167 | 0.330446 | NO |
| EGFR | 0.145898 | 0.183089 | 0.3197 | 0.357336 | NO |
| DUFD1 | 0.378603 | 0.985614 | 0.0888335 | 0.359478 | YES |
| MYBL2 | 0.0399249 | 0.176578 | 0.0716375 | 0.361422 | YES |
| S100A11 | 0.34613 | 0.556875 | 0.230849 | 0.363064 | NO |
| XBP1 | 0.045776 | 0.268606 | 0.0926021 | 0.400871 | NO |
| TOP2A | 0.240971 | 0.655786 | 0.0969129 | 0.404568 | YES |
| KIAA0310 | 0.484382 | 0.772587 | 0.342042 | 0.406749 | NO |
| KRT5 | 0.985088 | 0.984712 | 0.641471 | 0.409027 | NO |
| BF | 0.046196 | 0.204647 | 0.105472 | 0.463932 | NO |
| GSTP1 | 0.687906 | 0.677131 | 0.557251 | 0.465849 | NO |
| FZD7 | 0.594194 | 0.90597 | 0.384141 | 0.47759 | NO |
| NEK2 | 0.46014 | 0.932809 | 0.172718 | 0.500592 | YES |
| TAP1 | 0.663093 | 0.482788 | 0.541857 | 0.534398 | NO |
| FLJ14525 | 0.17537 | 0.17907 | 0.613531 | 0.561022 | NO |
| ACADSB | 0.0698192 | 0.387308 | 0.118621 | 0.576123 | NO |
| GARS | 0.709987 | 0.923267 | 0.902252 | 0.630522 | NO |
| BIRC5 | 0.397737 | 0.975853 | 0.170876 | 0.632892 | YES |
| HSD17B4 | 0.206242 | 0.395994 | 0.305472 | 0.635554 | NO |
| MKI67 | 0.311764 | 0.709371 | 0.195635 | 0.640833 | YES |
| PCNA | 0.868635 | 0.731512 | 0.557926 | 0.645851 | YES |
| PGR | 0.355079 | 0.965257 | 0.181127 | 0.681739 | NO |
| RABEP1 | 0.543773 | 0.963589 | 0.377702 | 0.682359 | NO |
| SLC7A6 | 0.432451 | 0.689547 | 0.419107 | 0.685462 | NO |
| SDC2 | 0.47607 | 0.37331 | 0.914923 | 0.689713 | NO |
| CKS2 | 0.936337 | 0.36756 | 0.180917 | 0.763492 | YES |
| DP1 | 0.149164 | 0.576409 | 0.32648 | 0.839276 | NO |
| CENPF | 0.19591 | 0.730895 | 0.203913 | 0.8435 | YES |
| CDK2AP1 | 0.711736 | 0.908545 | 0.835195 | 0.883836 | NO |
| RARRES3 | 0.0189691 | 0.107372 | 0.398642 | 0.943889 | NO |

TABLE 12

| Rank | UGCluster | Symbol | Gene Name |
|---|---|---|---|
| 1 | Hs.163484 | FOXA1 | Forkhead box A1 \|\| NM_004496 \|\| 14q12-q13 |
| 2 | Hs.446352 | ERBB2 | V-erb-b2 erythroblastic leukemia viral oncogene homolog 2, neuro/glioblastoma derived oncogene homolog |
| | | | (avian) \|\| NM_001005862 \|\| 17q11.2-q12 |
| 3 | Hs.496240 | AR | Androgen receptor (dihydrotestosterone receptor; testicular feminization; spinal and bulbar muscular atrophy; Kennedy |
| | | | disease) \|\| NM_000044 \|\| Xq11.2-q12 |
| 4 | Hs.387057 | FLJ13710 | Hypothetical protein FLJ13710 \|\| BX641106 \|\| 15q23 |
| 5 | Hs.437638 | XBP1 | X-box binding protein 1 \|\| AK093842 \|\| 22q12.1 |
| 6 | Hs.348883 | FOXC1 | Forkhead box C1 \|\| NM_001453 \|\| 6p25 |
| 7 | Hs.82961 | TFF3 | Trefoil factor 3 (intestinal) \|\| BU536516 \|\| 21q22.3 |
| 8 | Hs.155956 | NAT1 | N-acetyltransferase 1 (arylamine N-acetyltransferase) \|\| BC013732 \|\| 8p23.1-p21.3 |
| 9 | Hs.100686 | BCMP11 | Breast cancer membrane protein 11 \|\| BG540617 \|\| 7p21.1 |
| 10 | Hs.524134 | GATA3 | GATA binding protein 3 \|\| NM_001002295 \|\| 10p15 |
| 11 | Hs.530009 | AGR2 | Anterior gradient 2 homolog (Xenopus laevis) \|\| BM924878 \|\| 7p21.3 |
| 12 | Hs.208124 | ESR1 | Estrogen receptor 1 \|\| NM_000125 \|\| 6q25.1 |
| 13 | Hs.523468 | SCUBE2 | Signal peptide, CUB domain, EGF-like 2 \|\| NM_020974 \|\| 11p15.3 |
| 14 | Hs.469649 | BUB1 | BUB1 budding uninhibited by benzimidazoles 1 homolog (yeast) \|\| AF053305 \|\| 2q14 |
| 15 | Hs.79136 | SLC39A6 | Solute carrier family 39 (zinc transporter), member 6 \|\| NM_012319 \|\| 18q12.2 |
| 16 | Hs.144197 | UGTB | UDP glycosyltransferase 8 (UDP-galactose ceramide galactosyltransferase) \|\| NM_003360 \|\| 4q26 |
| 17 | Hs.27373 | LOC40045 | Hypothetical gene supported by AK075564; BC060873 \|\| NM_207446 \|\| 15q26.1 |
| 18 | Hs.414407 | KNTC2 | Kinetochore associated 2 \|\| NM_006101 \|\| 18p11.32 |
| 19 | Hs.115838 | TMC5 | Transmembrane channel-like 5 \|\| AY358155 \|\| 16p12.3 |
| 20 | Hs.210995 | CA12 | Carbonic anhydrase XII \|\| NM_001218 \|\| 15q22 |
| 21 | Hs.532968 | DKFZp762 | Hypothetical protein DKFZp762E1312 \|\| AK074809 \|\| 2q37.1 |
| 22 | Hs.514527 | BIRC5 | Baculoviral IAP repeat-containing 5 (survivin) \|\| NM_001012271 \|\| 17q25 |
| 23 | Hs.62180 | ANLN | Anillin, actin binding protein (scraps homolog, Drosophila) \|\| NM_018685 \|\| 7p15-p14 |
| 24 | Hs.14559 | C10orf3 | Chromosome 10 open reading frame 3 \|\| NM_018131 \|\| 10q23.33 |
| 25 | Hs.76277 | C19orf32 | Chromosome 19 open reading frame 32 \|\| BC008201 \|\| 19p13.3 |
| 26 | Hs.194698 | CCNB2 | Cyclin B2 \|\| AK023404 \|\| 15q22.2 |
| 27 | Hs.520189 | ELOVL5 | ELOVL family member 5, elongation of long chain fatty acids (FEN1/Elo2, SUR4/Elo3-like, |
| | | | yeast) \|\| AL833001 \|\| 6p21.1-p12.1 |
| 28 | Hs.504301 | LOC12022 | Transmembrane protein 45B \|\| NM_138788 \|\| 11q24.3 |
| 29 | Hs.169840 | TTK | TTK protein kinase \|\| NM_003318 \|\| 6q13-q21 |
| 30 | Hs.87417 | CTSL2 | Cathepsin L2 \|\| BC067289 \|\| 9q22.2 |
| 31 | Hs.1594 | CENPA | Centromere protein A, 17 kDa \|\| BM911202 \|\| 2p24-p21 |
| 32 | Hs.127407 | GALNT7 | UDP-N-acetyl-alpha-D-galactosamine:polypeptide N-acetylgalactosaminyltransferase 7 |
| | | | (GalNAc-T7) \|\| BC047468 \|\| 4q31.1 |
| 33 | Hs.260720 | DNAJC12 | DnaJ (Hsp40) homolog, subfamily C, member 12 \|\| NM_021800 \|\| 10q22.1 |
| 34 | Hs.102406 | MLPH | Melanophilin \|\| AK096789 \|\| 2q37.3 |
| 35 | Hs.692 | TACSTD1 | Tumor-associated calcium signal transducer 1 \|\| AK026585 \|\| 2p21 |
| 36 | Hs.524947 | CDC20 | CDC20 cell division cycle 20 homolog (S. cerevisiae) \|\| BG256659 \|\| 1p34.1 |
| 37 | Hs.99949 | PIP | Prolactin-induced protein \|\| BF965123 \|\| 7q34 |
| 38 | Hs.470654 | CDCA7 | Cell division cycle associated 7 \|\| AL834186 \|\| 2q31 |
| 39 | Hs.279651 | MIA | Melanoma inhibitory activity \|\| BG765502 \|\| 19q13.32-q13.33 |
| 40 | Hs.205952 | LOC20189 | Hypothetical protein LOC201895 \|\| BC047541 \|\| 4p14 |
| 41 | Hs.267659 | VAV3 | Vav 3 oncogene \|\| NM_006113 \|\| 1p13.3 |
| 42 | Hs.86859 | GRB7 | Growth factor receptor-bound protein 7 \|\| NM_005310 \|\| 17q12 |
| 43 | Hs.93002 | UBE2C | Ubiquitin-conjugating enzyme E2C \|\| BC032677 \|\| 20q13.12 |
| 44 | Hs.271224 | PH-4 | Hypoxia-inducible factor prolyl 4-hydroxylase \|\| NM_017732 \|\| 3p21.31 |
| 45 | Hs.24976 | ART3 | ADP-ribosyltransferase 3 \|\| AK129914 \|\| 4p15.1-p14 |
| 46 | Hs.184339 | MELK | Maternal embryonic leucine zipper kinase \|\| NM_014791 \|\| 9p13.2 |
| 47 | Hs.524571 | CDCA8 | Cell division cycle associated 8 \|\| BC000703 \|\| 1p34.3 |
| 48 | Hs.406050 | DNALI1 | Dynein, axonemal, light intermediate polypeptide 1 \|\| AK126963 \|\| 1p35.1 |
| 49 | Hs.152385 | FLJ10980 | Hypothetical protein FLJ10980 \|\| BC040548 \|\| 15q21.2-q21.3 |
| 50 | Hs.523220 | RAD54L | RAD54-like (S. cerevisiae) \|\| NM_003579 \|\| 1p32 |
| 51 | Hs.406013 | KRT18 | Keratin 18 \|\| CR616919 \|\| 12q13 |
| 52 | Hs.487036 | MYO5C | Myosin VC \|\| NM_018728 \|\| 15q21 |
| 53 | Hs.494496 | FBP1 | Fructose-1,6-bisphosphatase 1 \|\| AK223395 \|\| 9q22.3 |
| 54 | Hs.474217 | CDC45L | CDC45 cell division cycle 45-like (S. cerevisiae) \|\| BM478416 \|\| 22q11.21 |
| 55 | Hs.189119 | CXXC5 | CXXC finger 5 \|\| NM_016463 \|\| 5q31.2 |
| 56 | Hs.284153 | FANCA | Fanconi anemia, complementation group A \|\| NM_000135 \|\| 16q24.3 |
| 57 | Hs.531941 | MYB | V-myb myeloblastosis viral oncogene homolog (avian) \|\| AJ606319 \|\| 6q22-q23 |
| 58 | Hs.549195 | OGFRL1 | Opioid growth factor receptor-like 1 \|\| NM_024576 \|\| 6q13 |
| 59 | Hs.69360 | KIF2C | Kinesin family member 2C \|\| NM_006845 \|\| 1p34.1 |
| 60 | Hs.226390 | RRM2 | Ribonucleotide reductase M2 polypeptide \|\| AK123010 \|\| 2p25-p24 |
| 61 | Hs.250822 | STK6 | Serine/threonine Kinase 6 \|\| NM_198433 \|\| 20q13.2-q13.3 |
| 62 | Hs.490655 | ARP3BET [symbol] | Actin-related protein 3-beta \|\| AB209174 \|\| 7q32-q36 |
| 63 | Hs.516297 | TCF7L1 | Transcription factor 7-like 1 (T-cell specific, HMG-box) \|\| AK128630 \|\| 2p11.2 |
| 64 | Hs.252387 | CELSR1 | Cadherin, EGF LAG seven-pass G-type receptor 1 (flamingo homolog, Drosophila) \|\| AF231024 \|\| 22q13.3 |
| 65 | Hs.179718 | MYBL2 | V-myb myeloblastosis viral oncogene homolog (avian)-like 2 \|\| BX647151 \|\| 20q13.1 |
| 66 | Hs.201034 | NTN4 | Netrin 4 \|\| AF278532 \|\| 12q22-q23 |
| 67 | Hs.42645 | SLC16A6 | Solute carrier family 16 (monocarboxylic acid transporters), member 6 \|\| NM_004694 \|\| 17q24.2 |
| 68 | Hs.66762 | C10orf38 | Chromosome 10 open reading frame 38 \|\| AL050367 \|\| 10p13 |
| 69 | Hs.231320 | GPR160 | G protein-coupled receptor 160 \|\| AJ249248 \|\| 3q26.2-q27 |
| 70 | Hs.517549 | PIB5PA | Phosphatidylinositol (4,5) bisphosphate 5-phosphatase, A \|\| AK092859 \|\| 22q11.2-q13.2 |
| 71 | Hs.370549 | BCL11A | B-cell CLL/lymphoma 11A (zinc finger protein) \|\| NM_022893 \|\| 2p16.1 |
| 72 | Hs.96055 | E2F1 | E2F transcription factor 1 \|\| BC050369 \|\| 20q11.2 |
| 73 | Hs.505469 | RACGAP1 | Rac GTPase activating protein 1 \|\| NM_013277 \|\| 12q13.12 |
| 74 | Hs.436187 | TRIP13 | Thyroid hormone receptor interactor 13 \|\| NM_004237 \|\| 5p15.33 |
| 75 | Hs.5199 | HSPC150 | Ubiquitin-conjugating enzyme E2T (putative) \|\| BF690859 \|\| 1q32.1 |
| 76 | Hs.529181 | CAPN13 | Calpain 13 \|\| BX647678 \|\| |
| 77 | Hs.49433 | PTE2B | Peroxisomal acyl-CoA thioesterase 2B \|\| AK055797 \|\| 14q24.3 |
| 78 | Hs.459362 | PRC1 | Protein regulator of cytokinesis 1 \|\| NM_003981 \|\| 15q26.1 |
| 79 | Hs.485158 | SPDEF | SAM pointed domain containing ets transcription factor \|\| BC021299 \|\| 6p21.3 |
| 80 | Hs.262811 | KIAA1324 | Maba1 \|\| AB037745 \|\| 1p13.3 |
| 81 | Hs.213424 | SFRP1 | Secreted frizzled-related protein 1 \|\| BC036503 \|\| 8p12-p11.1 |
| 82 | Hs.364544 | TM4SF13 | Tetraspanin 13 \|\| AK128509 \|\| 7p21.1 |
| 83 | Hs.533185 | MAD2L1 | MAD2 mitotic arrest deficient-like 1 (yeast) \|\| BQ215664 \|\| 4q27 |
| 84 | Hs.153704 | NEK2 | NIMA (never in mitosis gene a)-related kinase 2 \|\| BC043502 \|\| 1q32.2-q41 |
| 85 | Hs.105547 | NPDC1 | Neural proliferation, differentiation and control, 1 \|\| AK054950 \|\| 9q34.3 |
| 86 | Hs.489353 | GPSM2 | G-protein signalling modulator 2 (AGS3-like, C. elegans) \|\| NM_013296 \|\| 1p13.3 |
| 87 | Hs.77695 | DLG7 | Discs, large homolog 7 (Drosophila) \|\| NM_014750 \|\| 14q22.3 |
| 88 | Hs.529285 | SLC40A1 | Solute carrier family 40 (iron-regulated transporter), member 1 \|\| NM_014585 \|\| 2q32 |
| 89 | Hs.49760 | ORC6L | Origin recognition complex, subunit 6 homolog-like (yeast) \|\| NM_014321 \|\| 16q12 |
| 90 | Hs.498248 | EXO1 | Exonuclease 1 \|\| NM_130398 \|\| 1q42-q43 |
| 91 | Hs.73625 | KIF20A | Kinesin family member 20A \|\| AK025790 \|\| 5q31 |
| 92 | Hs.165904 | EPN3 | Epsin 3 \|\| AK000785 \|\| 17q21.33 |
| 93 | Hs.350966 | PTTG1 | Pituitary tumor-transforming 1 \|\| BQ278502 \|\| 5q35.1 |
| 94 | Hs.199487 | RERG | RAS-like, estrogen-regulated, growth inhibitor \|\| BC007997 \|\| 12p12.3 |
| 95 | Hs.351344 | TMEM25 | Transmembrane protein 25 \|\| AK124814 \|\| 11q23.3 |
| 96 | Hs.487296 | PHGDH | Phosphoglycerate dehydrogenase \|\| AK093306 \|\| 1p12 |
| 97 | Hs.396783 | SLC9A3R [symbol] | Solute carrier family 9 (sodium/hydrogen exchanger), isoform 3 regulator 1 \|\| BX648303 \|\| 17q25.1 |
| 98 | Hs.404323 | FLJ10156 | Family with sequence similarity 64, member A \|\| CR590914 \|\| 17p13.2 |
| 99 | Hs.269109 | SEMA3C | Sema domain, immunoglobulin domain (Ig), short basic domain, secreted, (semaphorin) |
| | | | 3C \|\| NM_006379 \|\| 7q21-q31. |
| 100 | Hs.234545 | CDCA1 | Cell division cycle associated 1 \|\| NM_145697 \|\| 1q23.3 |

[symbol]

indicates data missing or illegible when filed

TABLE 13

| 7P-SAM-Order | CLID | Gene Name |
|---|---|---|
| 1 | Hs.163484 | Forkhead box A1 |
| 2 | Hs.446352 | V-erb-b2 erythroblastic leukemia viral oncogene homolog 2, neuro/glioblastoma derived oncoge [symbol] |
| 3 | Hs.496240 | Androgen receptor (dihydrotestosterone receptor; testicular feminization; spinal and bulbar mus [symbol] |
| 4 | Hs.387057 | Hypothetical protein FLJ13710 |
| 5 | Hs.437638 | X-box binding protein 1 |
| 6 | Hs.348883 | Forkhead box C1 |
| 7 | Hs.82961 | Trefoil factor 3 (intestinal) |
| 8 | Hs.155956 | N-acetyltransferase 1 (arylamine N-acetyltransferase) |
| 9 | Hs.100686 | Breast cancer membrane protein 11 |
| 10 | Hs.524134 | GATA binding protein 3 |
| 11 | Hs.530009 | Anterior gradient 2 homolog (Xenopus laevis) |
| 12 | Hs.208124 | Estrogen receptor 1 |
| 13 | Hs.523468 | Signal peptide, CUB domain, EGF-like 2 |
| 14 | Hs.469649 | BUB1 budding uninhibited by benzimidazoles 1 homolog (yeast) |
| 15 | Hs.79136 | Solute carrier family 39 (zinc transporter), member 6 |
| 16 | Hs.144197 | UDP glycosyltransferase 8 (UDP-galactose ceramide galactosyltransferase) |
| 17 | Hs.27373 | Hypothetical gene supported by AK075564; BC060873 |
| 18 | Hs.414407 | Kinetochore associated 2 |
| 19 | Hs.115838 | Transmembrane channel-like 5 |
| 20 | Hs.210995 | Carbonic anhydrase XII |
| 21 | Hs.532968 | Hypothetical protein DKFZp762E1312 |
| 22 | Hs.514527 | Baculoviral IAP repeat-containing 5 (survivin) |
| 23 | Hs.62180 | Anillin, actin binding protein (scraps homolog, Drosophila) |
| 24 | Hs.14559 | Chromosome 10 open reading frame 3 |
| 25 | Hs.76277 | Chromosome 19 open reading frame 32 |
| 26 | Hs.194698 | Cyclin B2 |
| 27 | Hs.520189 | ELOVL family member 5, elongation of long chain fatty acids (FEN1/Elo2, SUR4/Elo3-like, yeast |
| 28 | Hs.504301 | Transmembrane protein 45B |
| 29 | Hs.169840 | TTK protein kinase |
| 30 | Hs.87417 | Cathepsin L2 |
| 31 | Hs.1594 | Centromere protein A, 17 kDa |
| 32 | Hs.127407 | UDP-N-acetyl-alpha-D-galactosamine:polypeptide N-acetylgalactosaminyltransferase 7 (GalNAc |
| 33 | Hs.260720 | DnaJ (Hsp40) homolog, subfamily C, member 12 |
| 34 | Hs.102406 | Melanophilin |
| 35 | Hs.692 | Tumor-associated calcium signal transducer 1 |
| 36 | Hs.524947 | CDC20 cell division cycle 20 homolog (S. cerevisiae) |
| 37 | Hs.99949 | Prolactin-induced protein |
| 38 | Hs.470654 | Cell division cycle associated 7 |
| 39 | Hs.279651 | Melanoma inhibitory activity |
| 40 | Hs.205952 | Hypothetical protein LOC201895 |
| 41 | Hs.267659 | Vav 3 oncogene |
| 42 | Hs.86859 | Growth factor receptor-bound protein 7 |
| 43 | Hs.93002 | Ubiquitin-conjugating enzyme E2C |
| 44 | Hs.271224 | Hypoxia-inducible factor prolyl 4-hydroxylase |
| 45 | Hs.24976 | ADP-ribosyltransferase 3 |
| 46 | Hs.184339 | Maternal embryonic leucine zipper kinase |
| 47 | Hs.524571 | Cell division cycle associated 8 |
| 48 | Hs.406050 | Dynein, axonemal, light intermediate polypeptide 1 |
| 49 | Hs.152385 | Hypothetical protein FLJ10980 |
| 50 | Hs.523220 | RAD54-like (S. cerevisiae) |
| 51 | Hs.406013 | Keratin 18 |
| 52 | Hs.487036 | Myosin VC |
| 53 | Hs.494496 | Fructose-1,6-bisphosphatase 1 |
| 54 | Hs.474217 | CDC45 cell division cycle 45-like (S. cerevisiae) |
| 55 | Hs.189119 | CXXC finger 5 |
| 56 | Hs.284153 | Fanconi anemia, complementation group A |
| 57 | Hs.531941 | V-myb myeloblastosis viral oncogene homolog (avian) |
| 58 | Hs.549195 | Opioid growth factor receptor-like 1 |
| 59 | Hs.69360 | Kinesin family member 2C |
| 60 | Hs.226390 | Ribonucleotide reductase M2 polypeptide |
| 61 | Hs.250822 | Serine/threonine kinase 6 |
| 62 | Hs.490655 | Actin-related protein 3-beta |
| 63 | Hs.516297 | Transcription factor 7-like 1 (T-cell specific, HMG-box) |
| 64 | Hs.252387 | Cadherin, EGF LAG seven-pass G-type receptor 1 (flamingo homolog, Drosophila) |
| 65 | Hs.179718 | V-myb myeloblastosis viral oncogene homolog (avian)-like 2 |
| 66 | Hs.201034 | Netrin 4 |
| 67 | Hs.42645 | Solute carrier family 16 (monocarboxylic acid transporters), member 6 |
| 68 | Hs.66762 | Chromosome 10 open reading frame 38 |
| 69 | Hs.231320 | G protein-coupled receptor 160 |
| 70 | Hs.517549 | Phosphatidylinositol (4,5) bisphosphate 5-phosphatase, A |
| 71 | Hs.370549 | B-cell CLL/lymphoma 11A (zinc finger protein) |
| 72 | Hs.96055 | E2F transcription factor 1 |
| 73 | Hs.505469 | Rac GTPase activating protein 1 |
| 74 | Hs.436187 | Thyroid hormone receptor interactor 13 |
| 75 | Hs.5199 | Ubiquitin-conjugating enzyme E2T (putative) |
| 76 | Hs.529181 | Calpain 13 |
| 77 | Hs.49433 | Peroxisomal acyl-CoA thioesterase 2B |
| 78 | Hs.459362 | Protein regulator of cytokinesis 1 |
| 79 | Hs.485158 | SAM pointed domain containing ets transcription factor |
| 80 | Hs.262811 | Maba1 |
| 81 | Hs.213424 | Secreted frizzled-related protein 1 |
| 82 | Hs.364544 | Tetraspanin 13 |
| 83 | Hs.533185 | MAD2 mitotic arrest deficient-like 1 (yeast) |
| 84 | Hs.153704 | NIMA (never in mitosis gene a)-related kinase 2 |
| 85 | Hs.105547 | Neural proliferation, differentiation and control, 1 |
| 86 | Hs.489353 | G-protein signalling modulator 2 (AGS3-like, C. elegans) |
| 87 | Hs.77695 | Discs, large homolog 7 (Drosophila) |
| 88 | Hs.529285 | Solute carrier family 40 (iron-regulated transporter), member 1 |
| 89 | Hs.49760 | Origin recognition complex, subunit 6 homolog-like (yeast) |
| 90 | Hs.498248 | Exonuclease 1 |
| 91 | Hs.73625 | Kinesin family member 20A |
| 92 | Hs.165904 | Epsin 3 |
| 93 | Hs.350966 | Pituitary tumor-transforming 1 |
| 94 | Hs.199487 | RAS-like, estrogen-regulated, growth inhibitor |
| 95 | Hs.351344 | Transmembrane protein 25 |
| 96 | Hs.487296 | Phosphoglycerate dehydrogenase |
| 97 | Hs.396783 | Solute carrier family 9 (sodium/hydrogen exchanger), isoform 3 regulator 1 |
| 98 | Hs.404323 | Family with sequence similarity 64, member A |
| 99 | Hs.269109 | Sema domain, immunoglobulin domain (Ig), short basic domain, secreted, (semaphorin) 3C |
| 100 | Hs.234545 | Cell division cycle associated 1 |
| 101 | Hs.400556 | Breast carcinoma amplified sequence 1 |
| 102 | Hs.446438 | G protein-coupled receptor, family C, group 5, member C |
| 103 | Hs.518727 | RNA-binding protein |
| 104 | Hs.501309 | Cold inducible RNA binding protein |
| 105 | Hs.121028 | Asp (abnormal spindle)-like, microcephaly associated (Drosophila) |
| 106 | Hs.421956 | Spindle pole body component 25 homolog (S. cerevisiae) |
| 107 | Hs.155017 | Nuclear receptor interacting protein 1 |
| 108 | Hs.18268 | Adenylate kinase 5 |
| 109 | Hs.436912 | Kinesin family member C1 |
| 110 | Hs.226307 | Apolipoprotein B mRNA editing enzyme, catalytic polypeptide-like 3B |
| 111 | Hs.469199 | Ring finger protein 103 |
| 112 | Hs.13291 | Cyclin G2 |
| 113 | Hs.444637 | Low density lipoprotein receptor-related protein 8, apolipoprotein e receptor |
| 114 | Hs.1892 | Phenylethanolamine N-methyltransferase |
| 115 | Hs.534367 | Frizzled homolog 9 (Drosophila) |
| 116 | Hs.244580 | TPX2, microtubule-associated protein homolog (Xenopus laevis) |
| 117 | Hs.105887 | Similar to common salivary protein 1 |
| 118 | Hs.254414 | Serine-arginine repressor protein (35 kDa) |
| 119 | Hs.479220 | Prominin 1 |
| 120 | Hs.518055 | Leucine-rich repeats and immunoglobulin-like domains 1 |
| 121 | Hs.129691 | Zinc finger protein 552 |
| 122 | Hs.492261 | Tumor protein p53 inducible nuclear protein 1 |
| 123 | Hs.473695 | Chloride intracellular channel 6 |
| 124 | Hs.336768 | 4-aminobutyrate aminotransferase |
| 125 | Hs.351875 | Cytochrome c oxidase subunit VIc |
| 126 | Hs.335139 | Potassium channel tetramerisation domain containing 3 |
| 127 | Hs.10082 | Potassium intermediate/small conductance calcium-activated channel, subfamily N, member 4 |
| 128 | Hs.75438 | Quinoid dihydropteridine reductase |
| 129 | Hs.432638 | SRY (sex determining region Y)-box 11 |
| 130 | Hs.283749 | Angiogenin, ribonuclease, RNase A family, 5 |
| 131 | Hs.79741 | Likely ortholog of mouse dilute suppressor |
| 132 | Hs.473087 | CTP synthase |
| 133 | Hs.444082 | Enhancer of zeste homolog 2 (Drosophila) |
| 134 | Hs.11729 | Solute carrier family 27 (fatty acid transporter), member 2 |
| 135 | Hs.546241 | Complement component 4B |
| 136 | Hs.58650 | Hedgehog acyltransferase |
| 137 | Hs.95612 | Desmocollin 2 |
| 138 | Hs.519057 | Neuropeptide Y receptor Y1 |
| 139 | Hs.517860 | Chromosome 3 open reading frame 18 |
| 140 | Hs.239 | Forkhead box M1 |
| 141 | Hs.514033 | Sperm associated antigen 5 |
| 142 | Hs.129895 | T-box 3 (ulnar mammary syndrome) |
| 143 | Hs.252712 | Karyopherin alpha 2 (RAG cohort 1, Importin alpha 1) |
| 144 | Hs.458304 | Ropporin, rhophilin associated protein 1 |
| 145 | Hs.446554 | RAD51 homolog (RecA homolog, E. coli) (S. cerevisiae) |
| 146 | Hs.283532 | Uncharacterized bone marrow protein BM039 |
| 147 | Hs.487046 | Superoxide dismutase 2, mitochondrial |
| 148 | Hs.522665 | Melanoma antigen family D, 2 |
| 149 | Hs.514146 | Titin-cap (telethonin) |
| 150 | Hs.23960 | Cyclin B1 |
| 151 | Hs.434604 | Similar to ovostatin-2 |
| 152 | Hs.390788 | Protein kinase, X-linked |
| 153 | Hs.33102 | Transcription factor AP-2 beta (activating enhancer binding protein 2 beta) |
| 154 | Hs.473583 | Nuclease sensitive element binding protein 1 |
| 155 | Hs.444767 | Kinesin family member 13B |
| 156 | Hs.514211 | Hypothetical protein MGC4251 |
| 157 | Hs.104741 | PDZ binding kinase |
| 158 | Hs.518997 | Hypothetical protein FLJ10901 |
| 159 | Hs.118552 | Heat shock 70 kDa protein 5 (glucose-regulated, protein, 78 kDa) binding protein 1 |
| 160 | Hs.2025 | Transforming growth factor, beta 3 |
| 161 | Hs.2006 | Glutathione S-transferase M3 (brain) |
| 162 | Hs.368072 | Progesterone receptor |
| 163 | Hs.413111 | Phospholipase C, gamma 2 (phosphatidylinositol-specific) |
| 164 | Hs.480837 | inositol polyphosphate-4-phosphatase, type II, 105 kDa |
| 165 | Hs.270845 | Kinesin family member 23 |
| 166 | Hs.215766 | GTP binding protein 4 |
| 167 | Hs.209983 | Stathmin 1/oncoprotein 18 |
| 168 | Hs.162807 | Trefoil factor 1 (breast cancer, estrogen-inducible sequence expressed in) |
| 169 | Hs.532803 | Hematological and neurological expressed 1 |
| 170 | Hs.233160 | Stanniocalcin 2 |
| 171 | Hs.445098 | DEP domain containing 1 |
| 172 | Hs.169348 | Bloom syndrome |
| 173 | Hs.515122 | Thymidine kinase 1, soluble |
| 174 | Hs.29724 | Pleckstrin homology domain containing, family F (with FYVE domain) member 2 |
| 175 | Hs.49143 | MKL/myocardin-like 2 |
| 176 | Hs.334562 | Cell division cycle 2, G1 to S and G2 to M |
| 177 | Hs.511755 | Pituitary tumor-transforming 2 |
| 178 | Hs.212088 | Epoxide hydrolase 2, cytoplasmic |
| 179 | Hs.25318 | RAB27B, member RAS oncogene family |
| 180 | Hs.462998 | Insulin-like growth factor binding protein 4 |
| 181 | Hs.268728 | Tweety homolog 1 (Drosophila) |
| 182 | Hs.258326 | B/K protein |
| 183 | Hs.525419 | Epithelial protein lost in neoplasm beta |
| 184 | Hs.122908 | DNA replication factor |
| 185 | Hs.12970 | Proteasome (prosome, macropain) 26S subunit, non-ATPase, 3 |
| 186 | Hs.417962 | Dual specificity phosphatase 4 |
| 187 | Hs.211511 | Hypothetical protein FLJ21127 |
| 188 | Hs.444959 | Acyl-Coenzyme A oxidase 2, branched chain |
| 189 | Hs.323583 | Hypothetical protein DKFZp434L142 |
| 190 | Hs.119192 | H2A histone family, member Z |
| 191 | Hs.95243 | Transcription elongation factor A (SII)-like 1 |
| 192 | Hs.190440 | ADP-ribosylation factor-like 6 interacting protein 2 |
| 193 | Hs.444915 | Solute carrier family 1 (neuronal/epithelial high affinity glutamate transporter, system Xag), mem |
| 194 | Hs.326391 | Phytanoyl-CoA dioxygenase domain containing 1 |
| 195 | Hs.443861 | SFRS protein kinase 1 |
| 196 | Hs.159142 | Lunatic fringe homolog (Drosophila) |
| 197 | Hs.272416 | SID1 transmembrane family, member 1 |
| 198 | Hs.374613 | Inositol 1,4,5-triphosphate receptor, type 1 |
| 199 | Hs.129452 | Dachshund homolog 1 (Drosophila) |
| 200 | Hs.489207 | Asparagine synthetase |
| 201 | Hs.270833 | Amphiregulin (schwannoma-derived growth factor) |
| 202 | Hs.446680 | Retinoic acid induced 2 |
| 203 | Hs.190284 | Smith-Magenis syndrome chromosome region, candidate 6 |
| 204 | Hs.404741 | Nuclear factor (erythroid-derived 2)-like 3 |
| 205 | Hs.444118 | MCM6 minichromosome maintenance deficient 6 (MIS5 homolog, S. pombe) (S. cerevisiae) |
| 206 | Hs.527295 | Ectonucleotide pyrophosphatase/phosphodiesterase 1 |
| 207 | Hs.98849 | Dynein, cytoplasmic, light polypeptide 2B |
| 208 | Hs.525198 | TAL1 (SCL) interrupting locus |
| 209 | Hs.471508 | Insulin receptor substrate 1 |
| 210 | Hs.435458 | SET binding protein 1 |
| 211 | Hs.15929 | Chromosome 6 open reading frame 211 |
| 212 | Hs.525796 | Chromosome 15 open reading frame 23 |
| 213 | Hs.514840 | Chitinase 3-like 2 |
| 214 | Hs.481307 | MLF1 interacting protein |
| 215 | Hs.408767 | Crystallin, alpha B |
| 216 | Hs.8878 | Kinesin family member 11 |
| 217 | Hs.201083 | Mal, T-cell differentiation protein 2 |
| 218 | Hs.507669 | Hypothetical protein CG003 |
| 219 | Hs.118722 | Fucosyltransferase 8 (alpha (1,6) fucosyltransferase) |
| 220 | Hs.388255 | DC13 protein |
| 221 | Hs.491148 | Pericentriolar material 1 |
| 222 | Hs.38972 | Tetraspanin 1 |
| 223 | Hs.494261 | Phosphoserine aminotransferase 1 |
| 224 | Hs.465413 | Cytochrome b-5 |
| 225 | Hs.121536 | Family with sequence similarity 54, member A |
| 226 | Hs.369752 | Thymidylate synthetase |
| 227 | Hs.368260 | Hypothetical protein MGC11242 |
| 228 | Hs.19114 | High-mobility group box 3 |
| 229 | Hs.3041 | Uracil-DNA glycosylase 2 |
| 230 | Hs.15250 | Peroxisomal D3,D2-enoyl-CoA isomerase |
| 231 | Hs.191842 | Cadherin 3, type 1, P-cadherin (placental) |
| 232 | Hs.491757 | V-yes-1 Yamaguchi sarcoma viral related oncogene homolog |
| 233 | Hs.461086 | Cadherin 1, type 1, E-cadherin (epithelial) |
| 234 | Hs.368884 | Chromosome 2 open reading frame 23 |
| 235 | Hs.89603 | Mucin 1, transmembrane |
| 236 | Hs.473420 | BTG family, member 3 |
| 237 | Hs.533782 | Keratin 8 |
| 238 | Hs.518448 | Lysosomal-associated membrane protein 3 |
| 239 | Hs.507290 | Kelch/ankyrin repeat containing cyclin A1 interacting protein |
| 240 | Hs.445898 | V-myb myeloblastosis viral oncogene homolog (avian)-like 1 |
| 241 | Hs.486354 | Protein kinase (cAMP-dependent, catalytic) inhibitor beta |
| 242 | Hs.511093 | Nucleolar and spindle associated protein 1 |
| 243 | Hs.69089 | Galactosidase, alpha |
| 244 | Hs.523968 | Tumor protein p53 binding protein, 2 |
| 245 | Hs.546382 | Transcription factor CP2-like 2 |
| 246 | Hs.487200 | SPARC related modular calcium binding 2 |
| 247 | Hs.519601 | Inhibitor of DNA binding 4, dominant negative helix-loop-helix protein |
| 248 | Hs.435711 | Tripartite motif-containing 2 |
| 249 | Hs.79361 | Kallikrein 6 (neurosin, zyme) |
| 250 | Hs.534313 | Early growth response 3 |
| 251 | Hs.370834 | ATPase family, AAA domain containing 2 |
| 252 | Hs.3346 | Family with sequence similarity 63, member A |
| 253 | Hs.432448 | Keratin 16 (focal non-epidermolytic palmoplantar keratoderma) |
| 254 | Hs.405619 | Phosphoribosyl transferase domain containing 1 |
| 255 | Hs.145932 | Metallothionein-like 5, testis-specific (tesmin) |
| 256 | Hs.298654 | Dual specificity phosphatase 6 |
| 257 | Hs.519162 | BTG family, member 2 |
| 258 | Hs.81934 | Acyl-Coenzyme A dehydrogenase, short/branched chain |
| 259 | Hs.27695 | Midline 1 (Opitz/BBB syndrome) |
| 260 | Hs.406515 | NAD(P)H dehydrogenase, quinone 1 |
| 261 | Hs.87247 | Harakiri, BCL2 interacting protein (contains only BH3 domain) |
| 262 | Hs.546434 | V-set domain containing T cell activation inhibitor 1 |
| 263 | Hs.9029 | Keratin 23 (histone deacetylase inducible) |
| 264 | Hs.71465 | Squalene epoxidase |
| 265 | Hs.293970 | Aldehyde dehydrogenase 6 family, member A1 |
| 266 | Hs.523836 | Glutathione S-transferase pi |
| 267 | Hs.306777 | Gasdermin-like |
| 268 | Hs.520942 | Claudin 4 |
| 269 | Hs.111732 | Fc fragment of IgG binding protein |
| 270 | Hs.149387 | Myosin VI |
| 271 | Hs.87752 | Moesin |
| 272 | Hs.85137 | Cyclin A2 |
| 273 | Hs.511978 | Huntingtin interacting protein K |
| 274 | Hs.503709 | Pro-oncosis receptor inducing membrane injury gene |
| 275 | Hs.332847 | Cysteine-rich motor neuron 1 |
| 276 | Hs.197922 | Calcium/calmodulin-dependent protein kinase II |
| 277 | Hs.188606 | START domain containing 10 |
| 278 | Hs.6147 | Tensin like C1 domain containing phosphatase |
| 279 | Hs.367992 | Inositol(myo)-1(or 4)-monophosphatase 2 |
| 280 | Hs.503134 | 7-dehydrocholesterol reductase |
| 281 | Hs.458360 | Uridine-cytidine kinase 2 |
| 282 | Hs.491582 | Plasminogen activator, tissue |
| 283 | Hs.439760 | Cytochrome P450, family 4, subfamily X, polypeptide 1 |
| 284 | Hs.74120 | Chromosome 10 open reading frame 116 |
| 285 | Hs.58756 | Period homolog 2 (Drosophila) |
| 286 | Hs.372924 | CAMP responsive element binding protein 3-like 4 |
| 287 | Hs.508284 | F-box and leucine-rich repeat protein 3 |
| 288 | Hs.35086 | Ubiquitin specific protease 1 |
| 289 | Hs.121520 | Amphoterin induced gene 2 |
| 290 | Hs.173859 | Frizzled homolog 7 (Drosophila) |
| 291 | Hs.440494 | Chemokine-like factor super family 7 |
| 292 | Hs.67726 | Macrophage receptor with collagenous structure |
| 293 | Hs.200227 | FYVE and coiled-coil domain containing 1 |
| 294 | Hs.28309 | UDP-glucose dehydrogenase |
| 295 | Hs.438064 | FN5 protein |
| 296 | Hs.488143 | Biliverdin reductase A |
| 297 | Hs.370379 | Zinc finger protein 462 |
| 298 | Hs.406061 | Fatty acid binding protein 5 (psoriasis-associated) |
| 299 | Hs.444924 | CDP-diacylglycerol synthase (phosphatidate cytidylyltransferase) 1 |
| 300 | Hs.505575 | UDP-N-acetyl-alpha-D-galactosamine:polypeptide N-acetylgalactosaminyltransferase 6 (GalNAc |
| 301 | Hs.89626 | Parathyroid hormone-like hormone |
| 302 | Hs.518475 | Replication factor C (activator 1) 4, 37 kDa |
| 303 | Hs.24529 | CHK1 checkpoint homolog (S. pombe) |
| 304 | Hs.515258 | Growth differentiation factor 15 |
| 305 | Hs.103755 | Receptor-interacting serine-threonine kinase 2 |
| 306 | Hs.390738 | Hypothetical protein FLJ20366 |
| 307 | Hs.369520 | Synaptotagmin-like 2 |
| 308 | Hs.81131 | Guanldinoacetate N-methyltransferase |
| 309 | Hs.199338 | GLI-Kruppel family member GLI3 (Greig cephalopolysyndactyly syndrome) |
| 310 | Hs.56145 | Thymosin-like 8 |
| 311 | Hs.374378 | CDC28 protein kinase regulatory subunit 1B |
| 312 | Hs.145209 | Ubiquitin protein ligase E3 component n-recognin 1 |
| 313 | Hs.513053 | DnaJ (Hsp40) homolog, subfamily A, member 4 |
| 314 | Hs.8739 | Signal transducer and activator of transcription 3 interacting protein 1 |
| 315 | Hs.508141 | Diaphanous homolog 3 (Drosophila) |
| 316 | Hs.511739 | SUMO-1 activating enzyme subunit 2 |
| 317 | Hs.409662 | Collagen, type XIV, alpha 1 (undulin) |
| 318 | Hs.487561 | Islet cell autoantigen 1, 69 kDa |
| 319 | Hs.520973 | Heat shock 27 kDa protein 1 |
| 320 | Hs.31034 | Peroxisomal biogenesis factor 11A |
| 321 | Hs.311609 | DEAD (Asp-Glu-Ala-Asp) box polypeptide 39 |
| 322 | Hs.232543 | Programmed cell death 4 (neoplastic transformation inhibitor) |
| 323 | Hs.512599 | Cyclin-dependent kinase inhibitor 2A (melanoma, p16, inhibits CDK4) |
| 324 | Hs.272805 | HRAS-like suppressor 2 |
| 325 | Hs.75117 | Interleukin enhancer binding factor 2, 45 kDa |
| 326 | Hs.55279 | Serine (or cysteine) proteinase inhibitor, clade B (ovalbumin), member 5 |
| 327 | Hs.377484 | BCL2-associated athanogene |
| 328 | Hs.75360 | Carboxypeptidase E |
| 329 | Hs.83758 | CDC28 protein kinase regulatory subunit 2 |
| 330 | Hs.40403 | Cbp/p300-interacting transactivator, with Glu/Asp-rich carboxy-terminal domain, 1 |
| 331 | Hs.10649 | Chromosome 1 open reading frame 38 |
| 332 | Hs.446192 | Contactin associated protein-like 2 |
| 333 | Hs.382202 | Chitinase 3-like 1 (cartilage glycoprotein-39) |
| 334 | Hs.108029 | SH3 domain binding glutamic acid-rich protein like |
| 335 | Hs.477693 | NCK adaptor protein 1 |
| 336 | Hs.221941 | Cytochrome b reductase 1 |
| 337 | Hs.504115 | Tripartite motif-containing 29 |
| 338 | Hs.514470 | Solute carrier family 25 (mitochondrial deoxynucleotide carrier), member 19 |
| 339 | Hs.75238 | Chromatin assembly factor 1, subunit B (p60) |
| 340 | Hs.6980 | Aldo-keto reductase family 7, member A3 (aflatoxin aldehyde reductase) |
| 341 | Hs.62771 | Hypothetical protein FLJ20186 |
| 342 | Hs.433201 | CDK2-associated protein 1 |
| 343 | Hs.368254 | Homogentisate 1,2-dioxygenase (homogentisate oxidase) |
| 344 | Hs.32973 | Glycine receptor, beta |
| 345 | Hs.434255 | Pleckstrin and Sec7 domain containing 3 |
| 346 | Hs.14623 | Interferon, gamma-inducible protein 30 |
| 347 | Hs.430324 | Annexin A9 |
| 348 | Hs.233952 | Proteasome (prosome, macropain) subunit, alpha type, 7 |
| 349 | Hs.44278 | RAB17, member RAS oncogene family |
| 350 | Hs.522500 | KIAA0310 |
| 351 | Hs.533573 | CDC7 cell division cycle 7 (S. cerevisiae) |
| 352 | Hs.530024 | Chromosome 7 open reading frame 24 |
| 353 | Hs.520313 | CD164 antigen, siaiomucin |
| 354 | Hs.208912 | Chromosome 22 open reading frame 18 |
| 355 | Hs.444751 | PDZ domain containing 1 |
| 356 | Hs.126248 | Collagen, type IX, alpha 3 |
| 357 | Hs.81892 | KIAA0101 |
| 358 | Hs.416358 | Sal-like 2 (Drosophila) |
| 359 | Hs.508461 | Mitogen-activated protein kinase kinase kinase 1 |
| 360 | Hs.491172 | Neurobeachin |
| 361 | Hs.6434 | Chromosome 14 open reading frame 132 |
| 362 | Hs.376984 | SRY (sex determining region Y)-box 10 |
| 363 | Hs.525205 | NDRG family member 2 |
| 364 | Hs.520463 | PiggyBac transposable element derived 5 |
| 365 | Hs.104650 | Mago-nashi homolog |
| 366 | Hs.29802 | Slit homolog 2 (Drosophila) |
| 367 | Hs.84113 | Cyclin-dependent kinase inhibitor 3 (CDK2-associated dual specificity phosphatase) |
| 368 | Hs.42650 | ZW10 interactor |
| 369 | Hs.512732 | Nei endonuclease VIII-like 1 (E. coli) |
| 370 | Hs.525105 | SLIT and NTRK-like family, member 6 |
| 371 | Hs.497741 | Centromere protein F, 350/400ka (mitosin) |
| 372 | Hs.404321 | Glycyl-tRNA synthetase |
| 373 | Hs.546280 | Pentraxin-related gene, rapidly induced by IL-1 beta |
| 374 | Hs.118631 | Timeless homolog (Drosophila) |
| 375 | Hs.279766 | Kinesin family member 4A |
| 376 | Hs.244723 | Cyclin E1 |
| 377 | Hs.505934 | CGI-119 protein |
| 378 | Hs.409065 | Flap structure-specific endonuclease 1 |
| 379 | Hs.26770 | Fatty acid binding protein 7, brain |
| 380 | Hs.532265 | Gene model 83 |
| 381 | Hs.155597 | D component of complement (adipsin) |
| 382 | Hs.513141 | Isocitrate dehydrogenase 2 (NADP+), mitochondrial |
| 383 | Hs.484813 | DEK oncogene (DNA binding) |
| 384 | Hs.30824 | Leucine zipper transcription factor-like 1 |
| 385 | Hs.472010 | Prion protein (p27-30) (Creutzfeld-Jakob disease, Gerstmann-Strausler-Scheinker syndrome, fa [symbol] |
| 386 | Hs.42151 | Histamine N-methyltransferase |
| 387 | Hs.368433 | Tumor protein D52 |
| 388 | Hs.16064 | CNKSR family member 3 |
| 389 | Hs.7879 | Interferon-related developmental regulator 1 |
| 390 | Hs.519168 | Fibromodulin |
| 391 | Hs.524216 | Cell division cycle associated 3 |
| 392 | Hs.518602 | Wolfram syndrome 1 (wolframin) |
| 393 | Hs.460693 | Glutamic pyruvate transaminase (alanine aminotransferase) 2 |
| 394 | Hs.527412 | N-acylsphingosine amidohydrolase (acid ceramidase) 1 |
| 395 | Hs.131683 | Cytoplasmic polyadenylation element binding protein 3 |
| 396 | Hs.109425 | GDNF family receptor alpha 1 |
| 397 | Hs.153479 | Extra spindle poles like 1 (S. cerevisiae) |
| 398 | Hs.443551 | Hypothetical protein FLJ10706 |
| 399 | Hs.458485 | Interferon, alpha-inducible protein (clone IFI-15K) |
| 400 | Hs.370858 | Fucosidase, alpha-L-1, tissue |
| 401 | Hs.405958 | CDC6 cell division cycle 6 homolog (S. cerevisiae) |
| 402 | Hs.78619 | Gamma-glutamyl hydrolase (conjugase, folylpolygammaglutamyl hydrolase) |
| 403 | Hs.446149 | Lactate dehydrogenase B |
| 404 | Hs.8257 | Cytokine inducible SH2-containing protein |
| 405 | Hs.528721 | Sema domain, immunoglobulin domain (Ig), short basic domain, secreted, (semaphorin) 3E |
| 406 | Hs.160562 | Insulin-like growth factor 1 (somatomedin C) |
| 407 | Hs.552582 | Leucine rich repeat containing 17 |
| 408 | Hs.104019 | Transforming, acidic coiled-coil containing protein 3 |
| 409 | Hs.288856 | Prefoldin 5 |
| 410 | Hs.512636 | Proline-rich nuclear receptor coactivator 2 |
| 411 | Hs.517830 | Biotinidase |
| 412 | Hs.504550 | RAD51 associated protein 1 |
| 413 | Hs.82222 | Sema domain, immunoglobulin domain (Ig), short basic domain, secreted, (semaphorin) 3B |
| 414 | Hs.524195 | Rho GTPase activating protein 21 |
| 415 | Hs.460789 | Trinucleotide repeat containing 9 |
| 416 | Hs.81848 | RAD21 homolog (S. pombe) |
| 417 | Hs.369042 | Hypothetical protein FLJ20605 |
| 418 | Hs.268698 | Methylenetetrahydrofolate dehydrogenase (NADP+ dependent) 1-like |
| 419 | Hs.156519 | MutS homolog 2, colon cancer, nonpolyposis type 1 (E. coli) |
| 420 | Hs.279746 | Transient receptor potential cation channel, subfamily V, member 2 |
| 421 | Hs.184062 | Chromosome 20 open reading frame 24 |
| 422 | Hs.321541 | RAB11A, member RAS oncogene family |
| 423 | Hs.3352 | Histone deacetylase 2 |
| 424 | Hs.330384 | Coronin, actin binding protein, 1C |
| 425 | Hs.435730 | Iroquois homeobox protein 5 |
| 426 | Hs.528763 | Small nuclear ribonucleoprotein polypeptide A′ |
| 427 | Hs.531561 | Epithelial membrane protein 2 |
| 428 | Hs.470477 | Protein tyrosine phosphatase type IVA, member 2 |
| 429 | Hs.438720 | MCM7 minichromosome maintenance deficient 7 (S. cerevisiae) |
| 430 | Hs.191179 | RAB11 family interacting protein 1 (class I) |
| 431 | Hs.307905 | V-rel reticuloendotheliosis viral oncogene homolog B, nuclear factor of kappa light polypeptide g [symbol] |
| 432 | Hs.123253 | SHC SH2-domain binding protein 1 |
| 433 | Hs.508950 | Transglutaminase 1 (K polypeptide epidermal type I, protein-glutamine-gamma-glutamyltransfer [symbol] |
| 434 | Hs.191911 | Nuclear factor I/A |
| 435 | Hs.54470 | ATP-binding cassette, sub-family C (CFTR/MRP), member 8 |
| 436 | Hs.19545 | Frizzled homolog 4 (Drosophila) |
| 437 | Hs.111554 | ADP-ribosylation factor-like 7 |
| 438 | Hs.534385 | THO complex 4 |
| 439 | Hs.375707 | Coiled-coil-helix-coiled-coil-helix domain containing 5 |
| 440 | Hs.446850 | Chromosome 14 open reading frame 100 |
| 441 | Hs.89497 | Lamin B1 |
| 442 | Hs.444468 | CTD (carboxy-terminal domain, RNA polymerase II, polypeptide A) small phosphatase 1 |
| 443 | Hs.18349 | Mitochondrial ribosomal protein L15 |
| 444 | Hs.532491 | Cryptochrome 2 (photolyase-like) |
| 445 | Hs.510402 | Membrane cofactor protein (CD46, trophoblast-lymphocyte cross-reactive antigen) |
| 446 | Hs.524399 | Trophinin associated protein (tastin) |
| 447 | Hs.522730 | G protein-coupled receptor associated sorting protein 1 |
| 448 | Hs.275464 | Kallikrein 10 |
| 449 | Hs.123464 | Purinergic receptor P2Y, G-protein coupled, 5 |
| 450 | Hs.534293 | Serine (or cysteine) proteinase inhibitor, clade A (alpha-1 antiproteinase, antitrypsin), member 3 |
| 451 | Hs.303476 | Flavin containing monooxygenase 5 |
| 452 | Hs.479208 | F-box and leucine-rich repeat protein 5 |
| 453 | Hs.124165 | Mitochondrial ribosomal protein S30 |
| 454 | Hs.477481 | MCM2 minichromosome maintenance deficient 2, mitotin (S. cerevisiae) |
| 455 | Hs.510334 | Serine (or cysteine) proteinase inhibitor, clade A (alpha-1 antiproteinase, antitrypsin), member 5 |
| 456 | Hs.180535 | UNC-112 related protein 2 |
| 457 | Hs.12068 | Carnitine acetyltransferase |
| 458 | Hs.513726 | Guanylate binding protein 5 |
| 459 | Hs.72620 | Chromosome 20 open reading frame 28 |
| 460 | Hs.36794 | Cyclin D-type binding protein 1 |
| 461 | Hs.421907 | Glioma tumor suppressor candidate region gene 2 |
| 462 | Hs.479754 | V-kit Hardy-Zuckerman 4 feline sarcoma viral oncogene homolog |
| 463 | Hs.5719 | Chromosome condensation-related SMC-associated protein 1 |
| 464 | Hs.278857 | Heterogeneous nuclear ribonucleoprotein H2 (H′) |
| 465 | Hs.546324 | Guanine monphosphate synthetase |
| 466 | Hs.461647 | KIAA0182 protein |
| 467 | Hs.433180 | DNA replication complex GINS protein PSF2 |
| 468 | Hs.483026 | Praja 2, RING-H2 motif containing |
| 469 | Hs.449415 | Eukaryotic translation initiation factor 2C, 2 |
| 470 | Hs.272499 | Dehydrogenase/reductase (SDR family) member 2 |
| 471 | Hs.370927 | Hypothetical protein PRO1855 |
| 472 | Hs.546239 | Alpha-2-glycoprotein 1, zinc |
| 473 | Hs.101774 | Chromosome 20 open reading frame 23 |
| 474 | Hs.546418 | Zinc finger protein 339 |
| 475 | Hs.49688 | Actin binding LIM protein family, member 3 |
| 476 | Hs.188881 | Hypothetical protein FLJ34633 |
| 477 | Hs.72071 | Potassium channel tetramerisation domain containing 9 |
| 478 | Hs.75668 | Hypothetical protein FLJ14490 |
| 479 | Hs.433845 | Keratin 5 (epidermolysis bullosa simplex, Dowling-Meara/Kobner/Weber-Cockayne types) |
| 480 | Hs.514289 | Homeo box B2 |
| 481 | Hs.438533 | Polymerase (DNA directed) iota |
| 482 | Hs.133539 | Microtubule associated serine/threonine kinase family member 4 |
| 483 | Hs.413835 | Sin3-associated polypeptide, 30 kDa |
| 484 | Hs.54483 | N-myc (and STAT) interactor |
| 485 | Hs.183617 | Claudin 23 |
| 486 | Hs.173092 | Solute carrier family 24 (sodium/potassium/calcium exchanger), member 1 |
| 487 | Hs.533977 | Thioredoxin interacting protein |
| 488 | Hs.448520 | Solute carrier family 7 (cationic amino acid transporter, y+ system), member 2 |
| 489 | Hs.408658 | Cyclin E2 |
| 490 | Hs.374596 | Tumor protein, translationally-controlled 1 |
| 491 | Hs210532 | KIAA0141 |
| 492 | Hs.297304 | Glycosyltransferase 8 domain containing 1 |
| 493 | Hs.513915 | Claudin 7 |
| 494 | Hs.334832 | NAD(P)H:quinone oxidoreductase type 3, polypeptide A2 |
| 495 | Hs.500761 | Solute carrier family 16 (monocarboxylic acid transporters), member 3 |
| 496 | Hs.86368 | Calmegin |
| 497 | Hs.251673 | DNA (cytosine-5-)-methyltransferase 3 beta |
| 498 | Hs.4944 | Chromosome 9 open reading frame 58 |
| 499 | Hs.134434 | Ovo-like 1(Drosophila) |
| 500 | Hs.396393 | Ubiquitin-conjugating enzyme E2S |
| 501 | Hs.227817 | BCL2-related protein A1 |
| 502 | Hs.93304 | Phospholipase A2, group VII (platelet-activating factor acetylhydrolase, plasma) |
| 503 | Hs.181326 | Myotubularin related protein 2 |
| 504 | Hs.62661 | Guanylate binding protein 1, interferon-inducible, 67 kDa |
| 505 | Hs.517582 | MCM5 minichromosome maintenance deficient 5, cell division cycle 46 (S. cerevisiae) |
| 506 | Hs.7946 | Mitochondrial tumor suppressor 1 |
| 507 | Hs.159799 | Thyroid hormone receptor associated protein 2 |
| 508 | Hs.511668 | Vacuolar protein sorting 13C (yeast) |
| 509 | Hs.444448 | Potassium channel, subfamily K, member 5 |
| 510 | Hs.480938 | LPS-responsive vesicle trafficking, beach and anchor containing |
| 511 | Hs.334370 | Brain expressed, X-linked 1 |
| 512 | Hs.253594 | Trichorhinophalangeal syndrome I |
| 513 | Hs.533710 | Fibronectin leucine rich transmembrane protein 2 |
| 514 | Hs.307529 | Kinesin family member 15 |
| 515 | Hs.116651 | Epithelial V-like antigen 1 |
| 516 | Hs.375957 | Integrin, beta 2 (antigen CD18 (p95), lymphocyte function-associated antigen 1; macrophage ant |
| 517 | Hs.13155 | Integrin, beta 5 |
| 518 | Hs.507798 | Lipoma HMGIC fusion partner |
| 519 | Hs.109438 | Potassium channel tetramerisation domain containing 12 |
| 520 | Hs.477959 | Seven in absentia homolog 2 (Drosophila) |
| 521 | Hs.182014 | Hematopoietic protein 1 |
| 522 | Hs.25647 | V-fos FBJ murine osteosarcoma viral oncogene homolog |
| 523 | Hs.148989 | Cingulin-like 1 |
| 524 | Hs.112949 | Chromosome 1 open reading frame 34 |
| 525 | Hs.437474 | RIO kinase 1 (yeast) |
| 526 | Hs.425427 | Hypothetical protein FLJ20425 |
| 527 | Hs.517227 | Junctional adhesion molecule 2 |
| 528 | Hs.106880 | Bystin-like |
| 529 | Hs.12813 | TCDD-inducible poly(ADP-ribose) polymerase |
| 530 | Hs.274313 | Insulin-like growth factor binding protein 6 |
| 531 | Hs.325667 | Thrombospondin, type I, domain containing 1 |
| 532 | Hs.481571 | Ubiquinol-cytochrome c reductase hinge protein |
| 533 | Hs.35096 | Zinc finger and BTB domain containing 4 |
| 534 | Hs.167531 | Methylcrotonoyl-Coenzyme A carboxylase 2 (beta) |
| 535 | Hs.330663 | Hypothetical protein FLJ20641 |
| 536 | Hs.494321 | ATP/GTP binding protein 1 |
| 537 | Hs.440379 | Rho GTPase-activating protein |
| 538 | Hs.104570 | Kallikrein 8 (neuropsin/ovasin) |
| 539 | Hs.495728 | Plrin (iron-binding nuclear protein) |
| 540 | Hs.275775 | Selenoprotein P, plasma, 1 |
| 541 | Hs.74034 | Caveolin 1, caveolae protein, 22 kDa |
| 542 | Hs.9728 | Armadillo repeat containing, X-linked 1 |
| 543 | Hs.221889 | Cold shock domain protein A |
| 544 | Hs.422889 | Nudix (nucleoside diphosphate linked moiety X)-type motif 6 |
| 545 | Hs.431498 | Forkhead box P1 |
| 546 | Hs.171299 | Zinc finger and BTB domain containing 16 |
| 547 | Hs.59554 | Sestrin 1 |
| 548 | Hs.533738 | Hypothetical protein FLJ21827 |
| 549 | Hs.501513 | Comparative gene identification transcript 37 |
| 550 | Hs.188464 | Mannosidase, alpha, class 2B, member 2 |
| 551 | Hs.24719 | Modulator of apoptosis 1 |
| 552 | Hs.33455 | Peptidyl arginine deiminase, type II |
| 553 | Hs.549109 | Protein tyrosine phosphatase, receptor type, T |
| 554 | Hs.171825 | Basic helix-loop-helix domain containing, class B, 2 |
| 555 | Hs.108106 | Ubiquitin-like, containing PHD and RING finger domains, 1 |
| 556 | Hs.47166 | Chromosome 3 open reading frame 14 |
| 557 | Hs.293907 | Family with sequence similarity 38, member B |
| 558 | Hs.83381 | Guanine nucleotide binding protein (G protein), gamma 11 |
| 559 | Hs.447530 | Hyaluronan and proteoglycan link protein 3 |
| 560 | Hs.6985 | Matrilin 3 |
| 561 | Hs.75969 | Proline-rich nuclear receptor coactivator 1 |
| 562 | Hs.477869 | Phospholipid scramblase 4 |
| 563 | Hs.128196 | Hypothetical protein FLJ14966 |
| 564 | Hs.156346 | Topoisomerase (DNA) II alpha 170 kDa |
| 565 | Hs.109439 | Osteoglycin (osteoinductive factor, mimecan) |
| 566 | Hs.103147 | Sperm protein SSP411 |
| 567 | Hs.485640 | Primase, polypeptide 2A, 58 kDa |
| 568 | Hs.477789 | ATPase, Na+/K+ transporting, beta 3 polypeptide |
| 569 | Hs.518060 | ADP-ribosylation-like factor 6 interacting protein 6 |
| 570 | Hs.550539 | NudC domain containing 1 |
| 571 | Hs.471405 | Tubulin tyrosine ligase-like family, member 4 |
| 572 | Hs.467733 | GREB1 protein |
| 573 | Hs.509264 | Kelch domain containing 2 |
| 574 | Hs.500812 | Beta-transducin repeat containing |
| 575 | Hs.104320 | Golgi autoantigen, golgin subfamily a, 5 |
| 576 | Hs.87435 | Rho guanine exchange factor (GEF) 16 |
| 577 | Hs.549157 | Coenzyme Q4 homolog (yeast) |
| 578 | Hs.482976 | Hypothetical gene supported by AF038182; BC009203 |
| 579 | Hs.291030 | IQ motif containing GTPase activating protein 2 |
| 580 | Hs.496267 | Immunoglobulin (CD79A) binding protein 1 |
| 581 | Hs.530904 | Cysteine and glycine-rich protein 2 |
| 582 | Hs.506603 | DIP13 beta |
| 583 | Hs.30246 | Solute carrier family 19 (thiamine transporter), member 2 |
| 584 | Hs.328865 | Dynactin 4 (p62) |
| 585 | Hs.50915 | Kallikrein 5 |
| 586 | Hs.483444 | Chemokine (C—X—C motif) ligand 14 |
| 587 | Hs.494337 | Golgi phosphoprotein 2 |
| 588 | Hs.62128 | Trophoblast glycoprotein |
| 589 | Hs.147433 | Proliferating cell nuclear antigen |
| 590 | Hs.521459 | ADAM-like, decysin 1 |
| 591 | Hs.415762 | Lymphocyte antigen 6 complex, locus D |
| 592 | Hs.524161 | Ras suppressor protein 1 |
| 593 | Hs.18376 | Cingulin |
| 594 | Hs.529798 | Basic transcription factor 3 |
| 595 | Hs.510262 | Membrane targeting (tandem) C2 domain containing 1 |
| 596 | Hs.445052 | MutS homolog 6 (E. coli) |
| 597 | Hs.276905 | Microtubule associated serine/threonine kinase-like |
| 598 | Hs.105940 | Jerky homolog-like (mouse) |
| 599 | Hs.236774 | High mobility group nucleosomal binding domain 4 |
| 600 | Hs.386733 | Polyribonucleotide nucleotidyltransferase 1 |
| 601 | Hs.170673 | Epidermal retinal dehydrogenase 2 |
| 602 | Hs.49421 | Hypothetical protein FLJ23129 |
| 603 | Hs.297413 | Matrix metalloproteinase 9 (gelatinase B, 92 kDa gelatinase, 92 kDa type IV collagenase) |
| 604 | Hs.183109 | Monoamine oxidase A |
| 605 | Hs.494870 | B-box and SPRY domain containing |
| 606 | Hs.406861 | Hydroxysteroid (17-beta) dehydrogenase 4 |
| 607 | Hs.496646 | Interleukin 13 receptor, alpha 1 |
| 608 | Hs.239154 | Ankyrin repeat, family A (RFXANK-like), 2 |
| 609 | Hs.2128 | Dual specificity phosphatase 5 |
| 610 | Hs.26010 | Phosphofructokinase, platelet |
| 611 | Hs.508597 | Integrin, beta-like 1 (with EGF-like repeat domains) |
| 612 | Hs.435051 | Cyclin-dependent kinase Inhibitor 2D (p19, inhibits CDK4) |
| 613 | Hs.288996 | S100 calcium binding protein A14 |
| 614 | Hs.80976 | Antigen identified by monoclonal antibody KI-67 |
| 615 | Hs.512144 | Chromosome 6 open reading frame 66 |
| 616 | Hs.82028 | Transforming growth factor, beta receptor II (70/80 kDa) |
| 617 | Hs.386189 | G-2 and S-phase expressed 1 |
| 618 | Hs.368592 | Sortilin-related receptor, L(DLR class) A repeats-containing |
| 619 | Hs.495473 | Notch homolog 1, translocation-associated (Drosophila) |
| 620 | Hs.381099 | Lymphocyte cytosolic protein 1 (L-plastin) |
| 621 | Hs.304682 | Cystatin C (amyloid angiopathy and cerebral hemorrhage) |
| 622 | Hs.840 | Indoleamine-pyrrole 2,3 dioxygenase |
| 623 | Hs.66170 | SET and MYND domain containing 2 |
| 624 | Hs.525157 | Tumor necrosis factor (ligand) superfamily, member 13b |
| 625 | Hs.389374 | Hypothetical protein LOC257106 |
| 626 | Hs.173536 | Protein kinase D3 |
| 627 | Hs.656 | Cell division cycle 25C |
| 628 | Hs.486502 | Neuroblastoma RAS viral (v-ras) oncogene homolog |
| 629 | Hs.375108 | CD24 antigen (small cell lung carcinoma cluster 4 antigen) |
| 630 | Hs.473838 | Down syndrome critical region gene 2 |
| 631 | Hs.153692 | Monogenic, audiogenic seizure susceptibility 1 homolog (mouse) |
| 632 | Hs.62185 | Solute carrier family 9 (sodium/hydrogen exchanger), isoform 6 |
| 633 | Hs.553497 | Phosphatidylinositol glycan, class H |
| 634 | Hs.535901 | Block of proliferation 1 |
| 635 | Hs.498317 | CGI-146 protein |
| 636 | Hs.513439 | Galactosylceramidase (Krabbe disease) |
| 637 | Hs.17109 | Integral membrane protein 2A |
| 638 | Hs.348920 | FSH primary response (LRPR1 homolog, rat) 1 |
| 639 | Hs.25338 | Protease, serine, 23 |
| 640 | Hs.531550 | Transducer of ERBB2, 1 |
| 641 | Hs.482390 | Transforming growth factor, beta receptor III (betaglycan, 300 kDa) |
| 642 | Hs.72550 | Hyaluronan-mediated motility receptor (RHAMM) |
| 643 | Hs.416073 | S100 calcium binding protein A8 (calgranulin A) |
| 644 | Hs.424788 | Tetratricopeptide repeat domain 13 |
| 645 | Hs.534450 | ORM1-like 2 (S. cerevisiae) |
| 646 | Hs.368431 | Runt-related transcription factor 1; translocated to, 1 (cyclin D-related) |
| 647 | Hs.462983 | Thyroid hormone receptor associated protein 4 |
| 648 | Hs.315137 | Alanyl-tRNA synthetase |
| 649 | Hs.446450 | Integral membrane protein 2B |
| 650 | Hs.506748 | Hepatoma-derived growth factor (high-mobility group protein 1-like) |
| 651 | Hs.2316 | SRY (sex determining region Y)-box 9 (campomelic dysplasia, autosomal sex-reversal) |
| 652 | Hs.18676 | Sprouty homolog 2 (Drosophila) |
| 653 | Hs.172052 | Polo-like kinase 4 (Drosophila) |
| 654 | Hs.24950 | Regulator of G-protein signalling 5 |
| 655 | Hs.372688 | Rho-related BTB domain containing 2 |
| 656 | Hs.504765 | Ets variant gene 6 (TEL oncogene) |
| 657 | Hs.204096 | Secretoglobin, family 1D, member 2 |
| 658 | Hs.512973 | Butyrate-induced transcript 1 |
| 659 | Hs.494648 | Testis expressed sequence 10 |
| 660 | Hs.499000 | DnaJ (Hsp40) homolog, subfamily C, member 1 |
| 661 | Hs.114062 | Protein tyrosine phosphatase-like (proline instead of catalytic arginine), member a |
| 662 | Hs.522054 | Synaptotagmin-like 4 (granuphilin-a) |
| 663 | Hs.460095 | Chromosome 16 open reading frame 45 |
| 664 | Hs.161640 | Tyrosine aminotransferase |
| 665 | Hs.129758 | Proline-serine-threonine phosphatase interacting protein 1 |
| 666 | Hs.370359 | Nuclear factor I/B |
| 667 | Hs.533444 | 3-hydroxymethyl-3-methylglutaryl-Coenzyme A lyase (hydroxymethylglutaricaciduria) |
| 668 | Hs.476052 | SNF related kinase |
| 669 | Hs.24763 | RAN binding protein 1 |
| 670 | Hs.432548 | Chromosome 10 open reading frame 18 |
| 671 | Hs.150693 | Activated leukocyte cell adhesion molecule |
| 672 | Hs.274329 | TP53 activated protein 1 |
| 673 | Hs.193491 | Tubulin, beta 6 |
| 674 | Hs.414469 | Potassium voltage-gated channel, delayed-rectifier, subfamily S, member 3 |
| 675 | Hs.46720 | Transmembrane protease, serine 5 (spinesin) |
| 676 | Hs.549192 | Zinc finger, FYVE domain containing 21 |
| 677 | Hs.469030 | Methylenetetrahydrofolate dehydrogenase (NADP+ dependent) 2, methenyltetrahydrofolate cyclc [symbol] |
| 678 | Hs.438824 | CK2 interacting protein 1; HQ0024c protein |
| 679 | Hs.266728 | Hypothetical protein FLJ13639 |
| 680 | Hs.501574 | A disintegrin and metalloproteinase domain 8 |
| 681 | Hs.529846 | Calcium modulating ligand |
| 682 | Hs.497159 | Chromosome 1 open reading frame 21 |
| 683 | Hs.551530 | Trans-prenyltransferase |
| 684 | Hs.492314 | Lysosomal associated protein transmembrane 4 beta |
| 685 | Hs.530003 | Solute carrier family 2 (facilitated glucose/fructose transporter), member 5 |
| 686 | Hs.60339 | N-myristoyltransferase 2 |
| 687 | Hs.36761 | HRAS-like suppressor |
| 688 | Hs.442658 | Aurora kinase B |
| 689 | Hs.122514 | Mitochondrial solute carrier protein |
| 690 | Hs.514151 | ORM1-like 3 (S. cerevisiae) |
| 691 | Hs.520506 | F-box protein 5 |
| 692 | Hs.175322 | Ubiquitin specific protease 13 (isopeptidase T-3) |
| 693 | Hs.943 | Interleukin 32 |
| 694 | Hs.255973 | CREBBP/EP300 inhibitor 1 |
| 695 | Hs.346950 | Cellular retinoic acid binding protein 1 |
| 696 | Hs.183861 | Chromatin modifying protein 4C |
| 697 | Hs.276878 | Nucleoporin 93 kDa |
| 698 | Hs.18616 | Leucine zipper protein 5 |
| 699 | Hs.153752 | Cell division cycle 25B |
| 700 | Hs.378996 | Hyperparathyroidism 2 (with jaw tumor) |
| 701 | Hs.493309 | KIAA0020 |
| 702 | Hs.433839 | Eukaryotic translation elongation factor 1 alpha 2 |
| 703 | Hs.16184 | RAD17 homolog (S. pombe) |
| 704 | Hs.530331 | Pyruvate dehydrogenase (lipoamide) alpha 1 |
| 705 | Hs.440401 | All-trans-13,14-dihydroretinol saturase |
| 706 | Hs.79018 | Chromatin assembly factor 1, subunit A (p150) |
| 707 | Hs.531642 | RAB11 family interacting protein 3 (class II) |
| 708 | Hs.518608 | Morf4 family associated protein 1-like 1 |
| 709 | Hs.446685 | Peroxisomal long-chain acyl-coA thioesterase |
| 710 | Hs.523375 | KIAA0514 |
| 711 | Hs.301526 | Tripartite motif-containing 45 |
| 712 | Hs.413801 | Proteasome (prosome, macropain) activator subunit 4 |
| 713 | Hs.481720 | Myosin X |
| 714 | Hs.514167 | Keratin 19 |
| 715 | Hs.445890 | HSPC163 protein |
| 716 | Hs.34114 | ATPase, Na+/K+ transporting, alpha 2 (+) polypeptide |
| 717 | Hs.371021 | Lysosomal associated multispanning membrane protein 5 |
| 718 | Hs.188569 | Zinc finger, DHHC domain containing 13 |
| 719 | Hs.478150 | Programmed cell death 10 |
| 720 | Hs.434953 | High-mobility group box 2 |
| 721 | Hs.25640 | Claudin 3 |
| 722 | Hs.35198 | Ectonucleotide pyrophosphatase/phosphodiesterase 5 (putative function) |
| 723 | Hs.280932 | Peroxisomal biogenesis factor 7 |
| 724 | Hs.292097 | SEC15-like 1 (S. cerevisiae) |
| 725 | Hs.515100 | Peroxisomal biogenesis factor 11 gamma |
| 726 | Hs.520619 | Hypothetical protein FLJ20485 |
| 727 | Hs.269944 | Mitochondrial carrier homolog 2 (C. elegans) |
| 728 | Hs.336219 | Peroxisome biogenesis factor 13 |
| 729 | Hs.127032 | Relaxin 2 |
| 730 | Hs.515601 | Leukocyte immunoglobulin-like receptor, subfamily B (with TM and ITIM domains), member 6 |
| 731 | Hs.352018 | Transporter 1, ATP-binding cassette, sub-family B (MDR/TAP) |
| 732 | Hs.150718 | Junctional adhesion molecule 3 |
| 733 | Hs.486835 | Chromosome 6 open reading frame 96 |
| 734 | Hs.277035 | Monoglyceride lipase |
| 735 | Hs.326035 | Early growth response 1 |
| 736 | Hs.130759 | Phospholipid scramblase 1 |
| 737 | Hs.385861 | Kelch-like 7 (Drosophila) |
| 738 | Hs.498397 | CGI-49 protein |
| 739 | Hs.436367 | Laminin, alpha 3 |
| 740 | Hs.492555 | Enhancer of yellow 2 homolog (Drosophila) |
| 741 | Hs.528334 | Fatty acid amide hydrolase |
| 742 | Hs.546386 | Carbohydrate (chondroitin 4) sulfotransferase 11 |
| 743 | Hs.31439 | Serine protease inhibitor, Kunitz type, 2 |
| 744 | Hs.271135 | ATP synthase, H+ transporting, mitochondrial F1 complex, gamma polypeptide 1 |
| 745 | Hs.335614 | SEC14-like 2 (S. cerevisiae) |
| 746 | Hs.198363 | MCM10 minichromosome maintenance deficient 10 (S. cerevisiae) |
| 747 | Hs.80342 | Keratin 15 |
| 748 | Hs.224607 | Syndecan 1 |
| 749 | Hs.463421 | ATP-binding cassette, sub-family C (CFTR/MRP), member 3 |
| 750 | Hs.110675 | Apolipoprotein C-I |
| 751 | Hs.337295 | Stress-induced-phosphoprotein 1 (Hsp70/Hsp90-organizing protein) |
| 752 | Hs.130989 | Sodium channel, nonvoltage-gated 1 alpha |
| 753 | Hs.372914 | N-myc downstream regulated gene 1 |
| 754 | Hs.59757 | Zinc finger protein 281 |
| 755 | Hs.54697 | Cdc42 guanine nucleotide exchange factor (GEF) 9 |
| 756 | Hs.482625 | Cardiomyopathy associated 5 |
| 757 | Hs.94865 | TEA domain family member 4 |
| 758 | Hs.435063 | Rho GTPase activating protein 22 |
| 759 | Hs.524138 | Brain-specific angiogenesis inhibitor 2 |
| 760 | Hs.8859 | Calcium activated nucleotidase 1 |
| 761 | Hs.165195 | VAMP (vesicle-associated membrane protein)-associated protein A, 33 kDa |
| 762 | Hs.3416 | Adipose differentiation-related protein |
| 763 | Hs.497788 | Glutamyl-prolyl-tRNA synthetase |
| 764 | Hs.501140 | KIAA1598 |
| 765 | Hs.534395 | Plakophilin 3 |
| 766 | Hs.29068 | Hypothetical protein DKFZp761P0423 |
| 767 | Hs.187376 | Tetratricopeptide repeat domain 10 |
| 768 | Hs.530272 | Similar to RIKEN cDNA 5730528L13 gene |
| 769 | Hs.11463 | UMP-CMP kinase |
| 770 | Hs.476415 | Adaptor protein containing pH domain, PTB domain and leucine zipper motif 1 |
| 771 | Hs.522373 | Gelsolin (amyloidosis, Finnish type) |
| 772 | Hs.532359 | Ribosomal protein L5 |
| 773 | Hs.397638 | WD repeat domain 5 |
| 774 | Hs.466507 | Liver-specific bHLH-Zip transcription factor |
| 775 | Hs.533747 | Hypothetical protein MGC13183 |
| 776 | Hs.7736 | Mitochondrial ribosomal protein L27 |
| 777 | Hs.89404 | Msh homeo box homolog 2 (Drosophila) |
| 778 | Hs.518750 | OCIA domain containing 1 |
| 779 | Hs.78482 | Paralemmin |
| 780 | Hs.370457 | LETM1 domain containing 1 |
| 781 | Hs.1600 | Chaperonin containing TCP1, subunit 5 (epsilon) |
| 782 | Hs.6551 | ATPase, H+ transporting, lysosomal accessory protein 1 |
| 783 | Hs.211600 | Tumor necorsis factor, alpha-induced protein 3 |
| 784 | Hs.339811 | UDP glycosyltransferase 2 family, polypeptide B11 |
| 785 | Hs.1051 | Granzyme B (granzyme 2, cyctotoxic T-lymphocyte-associated serine esterase 1) |
| 786 | Hs.479491 | Putative NFkB activating protein 373 |
| 787 | Hs.461113 | Cirrhosis, autosomal recessive 1A (cirhin) |
| 788 | Hs.57652 | Cadherin, EGF LAG seven-pass G-type receptor 2 (flamingo homolog, Drosophila) |
| 789 | Hs.472847 | Chromosome 20 open reading frame 35 |
| 790 | Hs.445758 | E2F transcription factor 5, p130-binding |
| 791 | Hs.926 | Myxovirus (influenza virus) resistance 2 (mouse) |
| 792 | Hs.549043 | Insulin-like growth factor 2 (somatomedin A) |
| 793 | Hs.492516 | Prefoldin 2 |
| 794 | Hs.31564 | FRAS1 related extracellular matrix 1 |
| 795 | Hs.116935 | Zinc finger protein 521 |
| 796 | Hs.488173 | Hypothetical protein MGC7036 |
| 797 | Hs.546467 | Epithelial stromal interaction 1 (breast) |
| 798 | Hs.503860 | 6-pyruvoyltetrahydropterin synthase |
| 799 | Hs.75573 | Centromere protein E, 312 kDa |
| 800 | Hs.550491 | Histone 1, H2ak |
| 801 | Hs.47649 | Methylcrotonoyl-Coenzyme A carboxylase 1 (alpha) |
| 802 | Hs.398157 | Polo-like kinase 2 (Drosophila) |
| 803 | Hs.170019 | Runt-related transcription factor 3 |
| 804 | Hs.371013 | Jumonji domain containing 2B |
| 805 | Hs.266273 | Chromosome 20 open reading frame 172 |
| 806 | Hs.202453 | V-myc myelocytomatosis viral oncogene homolog (avian) |
| 807 | Hs.546305 | Transcription elongation factor B (SIII), polypeptide 1 (15 kDa, elongin C) |
| 808 | Hs.87016 | Hypothetical protein FLJ10647 |
| 809 | Hs.404802 | Histone deacetylase 11 |
| 810 | Hs.531668 | Chemokine (C-X3-C motif) ligand 1 |
| 811 | Hs.492407 | Tyrosine 3-monooxygenase/tryptophan-5-monooxygenase activation protein, zeta polypeptide |
| 812 | Hs.410497 | Brain protein J3 |
| 813 | Hs.369232 | Erythrocyte membrane protein band 4.1 like 5 |
| 814 | Hs.75367 | Src-like-adaptor |
| 815 | Hs.118681 | V-erb-b2 erythroblastic leukemia viral oncogene homolog 3 (avian) |
| 816 | Hs.458276 | Nuclear factor of kappa light polypeptide gene enhancer in B-cells inhibitor, epsilon |
| 817 | Hs.498115 | TAR (HIV) RNA binding protein 1 |
| 818 | Hs.87889 | Dicer1, Dcr-1 homolog (Drosophila) |
| 819 | Hs.56729 | Lymphocyte-specific protein 1 |
| 820 | Hs.317192 | DnaJ (Hsp40) homolog, subfamily B, member 11 |
| 821 | Hs.4747 | Dyskeratosis congenita 1, dyskerin |
| 822 | Hs.192854 | Rhotekin |
| 823 | Hs.444247 | Mst3 and SOK1-related kinase |
| 824 | Hs.181042 | Dmx-like 1 |
| 825 | Hs.171626 | S-phase kinase-associated protein 1A (p19A) |
| 826 | Hs.517586 | Myoglobin |
| 827 | Hs.406551 | Similar to RIKEN cDNA 4921524J17 |
| 828 | Hs.272848 | Hypothetical protein FLJ21019 |
| 829 | Hs.516633 | NCK-associated protein 1 |
| 830 | Hs.333823 | Mitochondrial ribosomal protein L13 |
| 831 | Hs.400095 | Heat shock 22 kDa protein 8 |
| 832 | Hs.386470 | Neuromedin B |
| 833 | Hs.380403 | Polycomb group ring finger 4 |
| 834 | Hs.201671 | SRY (sex determining region Y)-box 13 |
| 835 | Hs.333297 | Hypothetical protein LOC339745 |
| 836 | Hs.241575 | N-acetylglucosamine-1-phosphotransferase, gamma subunit |
| 837 | Hs.526735 | Zinc finger, MYND domain containing 10 |
| 838 | Hs.149443 | Cytochrome b-561 domain containing 2 |
| 839 | Hs.260041 | O-acetyltransferase |
| 840 | Hs.276770 | CD52 antigen (CAMPATH-1 antigen) |
| 841 | Hs.204749 | Protein tyrosine phosphatase, non-receptor type 14 |
| 842 | Hs.111903 | Fc fragment of IgG, receptor, transporter, alpha |
| 843 | Hs.505077 | Chromosome 12 open reading frame 11 |
| 844 | Hs.282984 | Dehydrogenase/reductase (SDR family) member 8 |
| 845 | Hs.283683 | Chromosome 8 open reading frame 4 |
| 846 | Hs.444028 | Cytoskeleton associated protein 2 |
| 847 | Hs.18442 | E-1 enzyme |
| 848 | Hs.127788 | Hypothetical protein FLJ12078 |
| 849 | Hs.11590 | Cathepsin F |
| 850 | Hs.26530 | Serum deprivation response (phosphatidylserine binding protein) |
| 851 | Hs.127799 | Baculoviral IAP repeat-containing 3 |
| 852 | Hs.525709 | Hypothetical protein FLJ20607 |
| 853 | Hs.237856 | Solute carrier family 15, member 3 |
| 854 | Hs.2785 | Keratin 17 |
| 855 | Hs.145575 | Ubiquitin-like 3 |
| 856 | Hs.22543 | Ubiquitin protein ligase E3A (human papilloma virus E6-associated protein, Angelman syndrom [symbol] |
| 857 | Hs.5210 | Gila maturation factor, gamma |
| 858 | Hs.408557 | Elongation of very long chain fatty acids (FEN1/Elo2, SUR4/Elo3, yeast)-like 2 |
| 859 | Hs.389438 | KIAA0590 gene product |
| 860 | Hs.412019 | Chromosome 6 open reading frame 80 |
| 861 | Hs.412842 | Chromosome 10 open reading frame 7 |
| 862 | Hs.308122 | Inositol 1,3,4-triphosphate 5/6 kinase |
| 863 | Hs.484738 | Myosin regulatory light chain interacting protein |
| 864 | Hs.182385 | Hepsin (transmembrane protease, serine 1) |
| 865 | Hs.446628 | Ribosomal protein S4, X-linked |
| 866 | Hs.376208 | Lymphotoxin beta (TNF superfamily, member 3) |
| 867 | Hs.435535 | Zinc finger protein 395 |
| 868 | Hs.438862 | EPS8-like 1 |
| 869 | Hs.103527 | SH2 domain protein 2A |
| 870 | Hs.122926 | Nuclear receptor subfamily 3, group C, member 1 (glucocorticoid receptor) |
| 871 | Hs.544577 | Glyceraldehyde-3-phosphate dehydrogenase |
| 872 | Hs.31130 | Transmembrane 7 superfamily member 2 |
| 873 | Hs.1908 | Proteoglycan 1, secretory granule |
| 874 | Hs.487471 | Hypothetical protein FLJ20171 |
| 875 | Hs.23198 | Chromosome 1 open reading frame 31 |
| 876 | Hs.149305 | Hypothetical protein MGC2603 |
| 877 | Hs.522891 | Chemokine (C—X—C motif) ligand 12 (stromal cell-derived factor 1) |
| 878 | Hs.58324 | A disintegrin-like and metalloprotease (reprolysin type) with thrombospondin type 1 motif, 5 (agg |
| 879 | Hs.88297 | Serine/threonine kinase 17b (apoptosis-inducing) |
| 880 | Hs.516777 | SH3-domain binding protein 4 |
| 881 | Hs.492869 | Family with sequence similarity 49, member B |
| 882 | Hs.508148 | Abl-interactor 1 |
| 883 | Hs.44298 | Mitochondrial ribosomal protein S17 |
| 884 | Hs.368610 | 3′-phosphoadenosine 5′-phosphosulfate synthase 1 |
| 885 | Hs.442657 | TBC1 domain family, member 8 (with GRAM domain) |
| 886 | Hs.473721 | Solute carrier family 2 (facilitated glucose transporter), member 1 |
| 887 | Hs.511425 | Signal recognition particle 9 kDa |
| 888 | Hs.515126 | Intercellular adhesion molecule 1 (CD54), human rhinovirus receptor |
| 889 | Hs.9315 | Olfactomedin-like 3 |
| 890 | Hs.533736 | RNA binding motif protein 7 |
| 891 | Hs.532253 | F-box protein 16 |
| 892 | Hs.298813 | Scavenger receptor class B, member 1 |
| 893 | Hs.110757 | DNA segment on chromosome 21 (unique) 2056 expressed sequence |
| 894 | Hs.48924 | Armadillo repeat containing, X-linked 2 |
| 895 | Hs.435560 | SCY1-like 3 (S. cerevisiae) |
| 896 | Hs.19492 | Protocadherin 8 |
| 897 | Hs.523852 | Cyclin D1 (PRAD1: parathyroid adenomatosis 1) |
| 898 | Hs.177576 | Hypothetical protein MGC52110 |
| 899 | Hs.549185 | PEST-containing nuclear protein |
| 900 | Hs.493919 | Myelin protein zero-like 1 |
| 901 | Hs.514718 | Chromosome 18 open reading frame 43 |
| 902 | Hs.171695 | Dual specificity phosphatase 1 |
| 903 | Hs.443057 | CD53 antigen |
| 904 | Hs.369779 | Sirtuin (silent mating type information regulation 2 homolog) 1 (S. cerevisiae) |
| 905 | Hs.458283 | Glutaredoxin 2 |
| 906 | Hs.519346 | Erbb2 interacting protein |
| 907 | Hs.695 | Cystatin B (stefin B) |
| 908 | Hs.241517 | Polymerase (DNA directed), theta |
| 909 | Hs.514681 | Mitogen-activated protein kinase kinase 4 |
| 910 | Hs.369089 | Collagen, type IV, alpha 5 (Alport syndrome) |
| 911 | Hs.171426 | Nuclear receptor coactivator 7 |
| 912 | Hs.481704 | Hypothetical protein FLJ20152 |
| 913 | Hs.146246 | Hypothetical protein MGC45780 |
| 914 | Hs.532851 | Ribonuclease H2, large subunit |
| 915 | Hs.308613 | CGI-12 protein |
| 916 | Hs.156316 | Decorin |
| 917 | Hs.104476 | Hypothetical protein MGC17299 |
| 918 | Hs.437388 | Phosphatidylinositol glycan, class T |
| 919 | Hs.246381 | CD68 antigen |
| 920 | Hs.2962 | S100 calcium binding protein P |
| 921 | Hs.518805 | High mobility group AT-hook 1 |
| 922 | Hs.438102 | Insulin-like growth factor binding protein 2, 36 kDa |
| 923 | Hs.418367 | Neuromedin U |
| 924 | Hs.396644 | Poly(A) binding protein interacting protein 2 |
| 925 | Hs.530792 | GTP cyclohydrolase I feedback regulator |
| 926 | Hs.154073 | Solute carrier family 35, member B1 |
| 927 | Hs.76057 | UDP-galactose-4-epimerase |
| 928 | Hs.211800 | Component of oligomeric golgi complex 2 |
| 929 | Hs.470943 | Signal transducer and activator of transcription 1, 91 kDa |
| 930 | Hs.54416 | Sine oculis homeobox homolog 1 (Drosophila) |
| 931 | Hs.293077 | Choline phosphotransferase 1 |
| 932 | Hs.435991 | Chromosome 4 open reading frame 16 |
| 933 | Hs.429666 | CCAAT/enhancer binding protein (C/EBP), gamma |
| 934 | Hs.513633 | G protein-coupled receptor 56 |
| 935 | Hs.46679 | CUE domain containing 1 |
| 936 | Hs.525232 | Low density lipoprotein receptor-related protein 10 |
| 937 | Hs.504641 | CD163 antigen |
| 938 | Hs.411641 | Eukaryotic translation initiation factor 4E binding protein 1 |
| 939 | Hs.125474 | Leupaxin |
| 940 | Hs.517307 | Myxovirus (influenza virus) resistance 1, interferon-inducible protein p78 (mouse) |
| 941 | Hs.146602 | Low molecular mass ubiquinone-binding protein (9.5 kD) |
| 942 | Hs.473117 | Chromosome 20 open reading frame 17 |
| 943 | Hs.124027 | Selenophosphate synthetase 1 |
| 944 | Hs.517603 | Manic fringe homolog (Drosophila) |
| 945 | Hs.15299 | HMBA-inducible |
| 946 | Hs.503721 | Dynein, cytoplasmic, heavy polypeptide 2 |
| 947 | Hs.77628 | START domain containing 3 |
| 948 | Hs.158529 | Calsyntenin 2 |
| 949 | Hs.443831 | Programmed cell death 5 |
| 950 | Hs.497183 | Influenza virus NS1A binding protein |
| 951 | Hs.480042 | Annexin A3 |
| 952 | Hs.512680 | C-type lectin domain family 11, member A |
| 953 | Hs.357567 | Hypothetical protein LOC130576 |
| 954 | Hs.212102 | Protein disulfide isomerase-associated 6 |
| 955 | Hs.512464 | Surfeit 1 |
| 956 | Hs.469615 | Septin 10 |
| 957 | Hs.520004 | Discoidin domain receptor family, member 1 |
| 958 | Hs.126774 | RA-regulated nuclear matrix-associated protein |
| 959 | Hs.127445 | Lipase A, lysosomal acid, cholesterol esterase (Wolman disease) |
| 960 | Hs.414362 | Cytochrome b5 reductase b5R.2 |
| 961 | Hs.483136 | COMM domain containing 10 |
| 962 | Hs.243678 | SRY (sex determining region Y)-box B |
| 963 | Hs.301350 | FXYD domain containing ion transport regulator 3 |
| 964 | Hs.407135 | Adenosine deaminase |
| 965 | Hs.184523 | Serine/threonine kinase 38 like |
| 966 | Hs.134665 | Schwannomin interacting protein 1 |
| 967 | Hs.412836 | Factor for adipocyte differentiation 15B |
| 968 | Hs.502338 | Solute carrier family 1 (glial high affinity glutamate transporter), member 2 |
| 969 | Hs.497599 | Tryptophanyl-tRNA synthetase |
| 970 | Hs.377972 | Chromosome 13 open reading frame 21 |
| 971 | Hs.132340 | Chromosome 6 open reading frame 85 |
| 972 | Hs.161220 | Hypothetical gene CG01B |
| 973 | Hs.471675 | Diacylglycerol kinase, delta 130 kDa |
| 974 | Hs.369022 | MOB1, Mps One Binder kinase activator-like 2B (yeast) |
| 975 | Hs.34871 | Zinc finger homeobox 1b |
| 976 | Hs.166551 | Chromosome 5 open reading frame 3 |
| 977 | Hs.90073 | CSE1 chromosome segregation 1-like (yeast) |
| 978 | Hs.546282 | Retinoblastoma binding protein 8 |
| 979 | Hs.508343 | Alpha-methylacyl-CoA racemase |
| 980 | Hs.248174 | Histone 1, H2ab |
| 981 | Hs.69771 | B-factor, properdin |
| 982 | Hs.83383 | Peroxiredoxin 4 |
| 983 | Hs.233119 | Malic enzyme 2, NAD(+)-dependent, mitochondrial |
| 984 | Hs.89545 | Proteasome (prosome, macropain) subunit, beta type, 4 |
| 985 | Hs.502756 | AHNAK nucleoprotein (desmoyokin) |
| 986 | Hs.227067 | ATPase family, AAA domain containing 3A |
| 987 | Hs.497684 | Protein phosphatase 2, regulatory subunit B (B56), alpha isoform |
| 988 | Hs.47357 | Cholesterol 25-hydroxylase |
| 989 | Hs.412707 | Hypoxanthine phosphoribosyltransferase 1 (Lesch-Nyhan syndrome) |
| 990 | Hs.55220 | BCL2-associated athanogene 2 |
| 991 | Hs.22891 | Solute carrier family 7 (cationic amino acid transporter, y+ system), member 8 |
| 992 | Hs.118183 | Hypothetical protein FLJ22833 |
| 993 | Hs.17558 | Hypothetical protein FLJ90586 |
| 994 | Hs.75093 | Procollagen-lysine 1, 2-oxoglutarate 5-dioxygenase 1 |
| 995 | Hs.159118 | Adenosylmethionine decarboxylase 1 |
| 996 | Hs.64016 | Protein S (alpha) |
| 997 | Hs.3109 | Rho GTPase activating protein 4 |
| 998 | Hs.471441 | Proteasome (prosome, macropain) subunit, beta type, 2 |
| 999 | Hs.104315 | Soc-2 suppressor of clear homolog (C. elegans) |
| 1000 | Hs.77448 | Aldehyde dehydrogenase 4 family, member A1 |
| 1001 | Hs.433203 | HSPC171 protein |
| 1002 | Hs.524763 | Two pore segment channel 1 |
| 1003 | Hs.50130 | Necdin homolog (mouse) |
| 1004 | Hs.345694 | Potassium channel modulatory factor 1 |
| 1005 | Hs.7917 | Likely ortholog of mouse hypoxia induced gene 1 |
| 1006 | Hs.482363 | Solute carrier family 30 (zinc transporter), member 5 |
| 1007 | Hs.181301 | Cathepsin S |
| 1008 | Hs.126688 | Choline dehydrogenase |
| 1009 | Hs.381178 | Breast carcinoma amplified sequence 4 |
| 1010 | Hs.426359 | DKFZp564J157 protein |
| 1011 | Hs.408528 | Retinoblastoma 1 (including osteosarcoma) |
| 1012 | Hs.435326 | Actin-like 6A |
| 1013 | Hs.21611 | Kinesin family member 3C |
| 1014 | Hs.502004 | Related RAS viral (r-ras) oncogene homolog 2 |
| 1015 | Hs.281898 | Absent in melanoma 2 |
| 1016 | Hs.483473 | Chromosome 5 open reading frame 5 |
| 1017 | Hs.16362 | Pyrimidinergic receptor P2Y, G-protein coupled, 6 |
| 1018 | Hs.511067 | Hypothetical protein FLJ10579 |
| 1019 | Hs.125867 | Enah/Vasp-like |
| 1020 | Hs.404088 | Sarcoma antigen NY-SAR-48 |
| 1021 | Hs.118554 | Lactamase, beta 2 |
| 1022 | Hs.432438 | Echinoderm microtubule associated protein like 4 |
| 1023 | Hs.517232 | Peroxisomal biogenesis factor 19 |
| 1024 | Hs.128686 | Nucleobindin 2 |
| 1025 | Hs.93832 | Putative membrane protein |
| 1026 | Hs.497200 | Phospholipase A2, group IVA (cytosolic, calcium-dependent) |
| 1027 | Hs.418123 | Cathepsin L |
| 1028 | Hs.485938 | Ras-related GTP binding D |
| 1029 | Hs.280342 | Protein kinase, cAMP-dependent, regulatory, type I, alpha (tissue specific extinguisher 1) |
| 1030 | Hs.1987 | CD28 antigen (Tp44) |
| 1031 | Hs.533628 | KIAA0133 |
| 1032 | Hs.337594 | Serine dehydratase-like |
| 1033 | Hs.83169 | Matrix metalloproteinase 1 (interstitial collagenase) |
| 1034 | Hs.470608 | Solute carrier family 25 (mitochondrial carrier, Aralar), member 12 |
| 1035 | Hs.282326 | Down syndrome critical region gene 1 |
| 1036 | Hs.517581 | Heme oxygenase (decycling) 1 |
| 1037 | Hs.95351 | Lipase, hormone-sensitive |
| 1038 | Hs.433512 | ARP3 actin-related protein 3 homolog (yeast) |
| 1039 | Hs.292156 | Dickkopf homolog 3 (Xenopus laevis) |
| 1040 | Hs.439726 | Laminin, beta 2 (laminin S) |
| 1041 | Hs.506325 | Nudix (nucleoside diphosphate linked moiety X)-type motif 4 |
| 1042 | Hs.50984 | Sarcoma amplified sequence |
| 1043 | Hs.476319 | Enoyl Coenzyme A hydratase domain containing 2 |
| 1044 | Hs.332706 | Optineurin |
| 1045 | Hs.105700 | Secreted frizzled-related protein 4 |
| 1046 | Hs.26403 | Glutathione transferase zeta 1 (maleylacetoacetate isomerase) |
| 1047 | Hs.476365 | Sterol carrier protein 2 |
| 1048 | Hs.533260 | KIAA0649 |
| 1049 | Hs.435661 | Serine palmitoyltransferase, long chain base subunit 2 |
| 1050 | Hs.459952 | Stannin |
| 1051 | Hs.97220 | Chondroadherin |
| 1052 | Hs.20013 | GCIP-interacting protein p29 |
| 1053 | Hs.194329 | Transcription elongation factor A (SII)-like 4 |
| 1054 | Hs.495710 | Glycoprotein M6B |
| 1055 | Hs.90753 | HIV-1 Tat interactive protein 2, 30 kDa |
| 1056 | Hs.411847 | Mitogen-activated protein kinase 6 |
| 1057 | Hs.78944 | Regulator of G protein signalling 2, 24 kDa |
| 1058 | Hs.467769 | Family with sequence similarity 49, member A |
| 1059 | Hs.188634 | Sorting nexin 1 |
| 1060 | Hs.107740 | Kruppel-like factor 2 (lung) |
| 1061 | Hs.534169 | Heat shock 70 kDa protein 14 |
| 1062 | Hs.508234 | Kruppel-like factor 5 (intestinal) |
| 1063 | Hs.128065 | Cathepsin C |
| 1064 | Hs.105153 | Shugoshin-like 1 (S. pombe) |
| 1065 | Hs.9088 | Ankyrin repeat domain 34 |
| 1066 | Hs.530157 | FP15737 |
| 1067 | Hs.173288 | SH2 domain binding protein 1 (tetratricopeptide repeat containing) |
| 1068 | Hs.515785 | Biliverdin reductase B (flavin reductase (NADPH)) |
| 1069 | Hs.493096 | Pre-B-cell leukemia transcription factor 1 |
| 1070 | Hs.8526 | UDP-GlcNAc:betaGal beta-1,3-N-acetylglucosaminyltransferase 6 |
| 1071 | Hs.497636 | Laminin, beta 3 |
| 1072 | Hs.491351 | Clathrin, heavy polypeptide (Hc) |
| 1073 | Hs.523332 | Ornithine aminotransferase (gyrate atrophy) |
| 1074 | Hs.477114 | Pleckstrin homology-like domain, family B, member 2 |
| 1075 | Hs.509966 | Chromosome 14 open reading frame 58 |
| 1076 | Hs.75069 | Serine hydroxymethyltransferase 2 (mitochondrial) |
| 1077 | Hs.523009 | Sparc/osteonectin, cwcv and kazal-like domains proteoglycan (testican) 2 |
| 1078 | Hs.8036 | MBC3205 |
| 1079 | Hs.549198 | F-box protein 31 |
| 1080 | Hs.532626 | MGC16028 similar to RIKEN cDNA 1700019E19 gene |
| 1081 | Hs.189075 | PTK9 protein tyrosine kinase 9 |
| 1082 | Hs.495912 | Dystrophin (muscular dystrophy, Duchenne and Becker types) |
| 1083 | Hs.421724 | Cathepsin G |
| 1084 | Hs.532815 | Elastin microfibril interface 2 |
| 1085 | Hs.477921 | WW domain containing transcription regulator 1 |
| 1086 | Hs.520494 | Hypothetical protein FLJ14525 |
| 1087 | Hs.31442 | RecQ protein-like 4 |
| 1088 | Hs.2030 | Thrombomodulin |
| 1089 | Hs.508480 | RAP2A, member of RAS oncogene family |
| 1090 | Hs.153678 | Reproduction 8 |
| 1091 | Hs.319334 | Nuclear autoantigenic sperm protein (histone-binding) |
| 1092 | Hs.396358 | Hypothetical protein FLJ11273 |
| 1093 | Hs.514402 | Hypothetical protein MGC10986 |
| 1094 | Hs.168950 | Ganglioside-induced differentiation associated protein 1 |
| 1095 | Hs.74576 | GDP dissociation inhibitor 1 |
| 1096 | Hs.310542 | Translocase of outer mitochondrial membrane 40 homolog (yeast) |
| 1097 | Hs.285976 | LAG1 longevity assurance homolog 2 (S. cerevisiae) |
| 1098 | Hs.519018 | SH3 domain protein D19 |
| 1099 | Hs.524464 | ATP synthase, H+ transporting, mitochondrial F0 complex, subunit c (subunit 9), isoform 2 |
| 1100 | Hs.369554 | Solute carrier family 16 (monocarboxylic acid transporters), member 5 |
| 1101 | Hs.513315 | Nudix (nucleoside diphosphate linked moiety X)-type motif 16-like 1 |
| 1102 | Hs.150595 | Cytochrome P450, family 26, subfamily A, polypeptide 1 |

| 7P-SAM-Order | Annotation |
|---|---|
| 1 | Forkhead box A1 \|\| NM_004496 \|\| 14q12-q13 |
| 2 | V-erb-b2 erythroblastic leukemia viral oncogene homolog 2, neuro/glioblastoma derived oncoge [symbol] |
| 3 | Androgen receptor (dihydrotestosterone receptor; testicular feminization; spinal and bulbar mus [symbol] |
| 4 | Hypothetical protein FLJ13710 \|\| BX641106 \|\| 15q23 |
| 5 | X-box binding protein 1 \|\| AK093842 \|\| 22q12.1 |
| 6 | Forkhead box C1 \|\| NM_001453 \|\| 6p25 |
| 7 | Trefoil factor 3 (intestinal) \|\| BU536516 \|\| 21q22.3 |
| 8 | N-acetyltransferase 1 (arylamine N-acetyltransferase) \|\| BC013732 \|\| 8p23.1-p21.3 |
| 9 | Breast cancer membrane protein 11 \|\| BG540617 \|\| 7p21.1 |
| 10 | GATA binding protein 3 \|\| NM_001002295 \|\| 10p15 |
| 11 | Anterior gradient 2 homolog (Xenopus laevis) \|\| BM924878 \|\| 7p21.3 |
| 12 | Estrogen receptor 1 \|\| NM_000125 \|\| 6q25.1 |
| 13 | Signal peptide, CUB domain, EGF-like 2 \|\| NM_020974 \|\| 11p15.3 |
| 14 | BUB1 budding uninhibited by benzimidazoles 1 homolog (yeast) \|\| AF053305 \|\| 2q14 |
| 15 | Solute carrier family 39 (zinc transporter), member 6 \|\| NM_012319 \|\| 18q12.2 |
| 16 | UDP glycosyltransferase 8 (UDP-galactose ceramide galactosyltransferase) \|\| NM_003360 \|\| 4q [symbol] |
| 17 | Hypothetical gene supported by AK075564; BC060873 \|\| NM_207446 \|\| 15q26.1 |
| 18 | Kinetochore associated 2 \|\| NM_006101 \|\| 18p11.32 |
| 19 | Transmembrane channel-like 5 \|\| AY358155 \|\| 16p12.3 |
| 20 | Carbonic anhydrase XII \|\| NM_001218 \|\| 15q22 |
| 21 | Hypothetical protein DKFZp762E1312 \|\| AK074809 \|\| 2q37.1 |
| 22 | Baculoviral IAP repeat-containing 5 (survivin) \|\| NM_001012271 \|\| 17q25 |
| 23 | Anillin, actin binding protein (scraps homolog, Drosophila) \|\| NM_018685 \|\| 7p15-p14 |
| 24 | Chromosome 10 open reading frame 3 \|\| NM_018131 \|\| 10q23.33 |
| 25 | Chromosome 19 open reading frame 32 \|\| BC008201 \|\| 19p13.3 |
| 26 | Cyclin B2 \|\| AK023404 \|\| 15q22.2 |
| 27 | ELOVL family member 5, elongation of long chain fatty acids (FEN1/Elo2, SUR4/Elo3-like, yeast |
| 28 | Transmembrane protein 45B \|\| NM_138788 \|\| 11q24.3 |
| 29 | TTK protein kinase \|\| NM_003318 \|\| 6q13-q21 |
| 30 | Cathepsin L2 \|\| BC067289 \|\| 9q22.2 |
| 31 | Centromere protein A, 17 kDa \|\| BM911202 \|\| 2p24-p21 |
| 32 | UDP-N-acetyl-alpha-D-galactosamine:polypeptide N-acetylgalactosaminyltransferase 7 (GalNAc |
| 33 | DnaJ (Hsp40) homolog, subfamily C, member 12 \|\| NM_021800 \|\| 10q22.1 |
| 34 | Melanophilin \|\| AK096789 \|\| 2q37.3 |
| 35 | Tumor-associated calcium signal transducer 1 \|\| AK026585 \|\| 2p21 |
| 36 | CDC20 cell division cycle 20 homolog (S. cerevisiae) \|\| BG256659 \|\| 1p34.1 |
| 37 | Prolactin-induced protein \|\| BF965123 \|\| 7q34 |
| 38 | Cell division cycle associated 7 \|\| AL834186 \|\| 2q31 |
| 39 | Melanoma inhibitory activity \|\| BG765502 \|\| 19q13.32-q13.33 |
| 40 | Hypothetical protein LOC201895 \|\| BC047541 \|\| 4p14 |
| 41 | Vav 3 oncogene \|\| NM_006113 \|\| 1p13.3 |
| 42 | Growth factor receptor-bound protein 7 \|\| NM_005310 \|\| 17q12 |
| 43 | Ubiquitin-conjugating enzyme E2C \|\| BC032677 \|\| 20q13.12 |
| 44 | Hypoxia-inducible factor prolyl 4-hydroxylase \|\| NM_017732 \|\| 3p21.31 |
| 45 | ADP-ribosyltransferase 3 \|\| AK129914 \|\| 4p15.1-p14 |
| 46 | Maternal embryonic leucine zipper kinase \|\| NM_014791 \|\| 9p13.2 |
| 47 | Cell division cycle associated 8 \|\| BC000703 \|\| 1p34.3 |
| 48 | Dynein, axonemal, light intermediate polypeptide 1 \|\| AK126963 \|\| 1p35.1 |
| 49 | Hypothetical protein FLJ10980 \|\| BC040548 \|\| 15q21.2-q21.3 |
| 50 | RAD54-like (S. cerevisiae) \|\| NM_003579 \|\| 1p32 |
| 51 | Keratin 18 \|\| CR616919 \|\| 12q13 |
| 52 | Myosin VC \|\| NM_018728 \|\| 15q21 |
| 53 | Fructose-1,6-bisphosphatase 1 \|\| AK223395 \|\| 9q22.3 |
| 54 | CDC45 cell division cycle 45-like (S. cerevisiae) \|\| BM478416 \|\| 22q11.21 |
| 55 | CXXC finger 5 \|\| NM_016463 \|\| 5q31.2 |
| 56 | Fanconi anemia, complementation group A \|\| NM_000135 \|\| 16q24.3 |
| 57 | V-myb myeloblastosis viral oncogene homolog (avian) \|\| AJ606319 \|\| 6q22-q23 |
| 58 | Opioid growth factor receptor-like 1 \|\| NM_024576 \|\| 6q13 |
| 59 | Kinesin family member 2C \|\| NM_006845 \|\| 1p34.1 |
| 60 | Ribonucleotide reductase M2 polypeptide \|\| AK123010 \|\| 2p25-p24 |
| 61 | Serine/threonine kinase 6 \|\| NM_198433 \|\| 20q13.2-q13.3 |
| 62 | Actin-related protein 3-beta \|\| AB209174 \|\| 7q32-q36 |
| 63 | Transcription factor 7-like 1 (T-cell specific, HMG-box) \|\| AK128630 \|\| 2p11.2 |
| 64 | Cadherin, EGF LAG seven-pass G-type receptor 1 (flamingo homolog, Drosophila) \|\| AF231024 |
| 65 | V-myb myeloblastosis viral oncogene homolog (avian)-like 2 \|\| BX647151 \|\| 20q13.1 |
| 66 | Netrin 4 \|\| AF278532 \|\| 12q22-q23 |
| 67 | Solute carrier family 16 (monocarboxylic acid transporters), member 6 \|\| NM_004594 \|\| 17q24.2 |
| 68 | Chromosome 10 open reading frame 38 \|\| AL050367 \|\| 10p13 |
| 69 | G protein-coupled receptor 160 \|\| AJ249248 \|\| 3q26.2-q27 |
| 70 | Phosphatidylinositol (4,5) bisphosphate 5-phosphatase, A \|\| AK092859 \|\| 22q11.2-q13.2 |
| 71 | B-cell CLL/lymphoma 11A (zinc finger protein) \|\| NM_022893 \|\| 2p16.1 |
| 72 | E2F transcription factor 1 \|\| BC050369 \|\| 20q11.2 |
| 73 | Rac GTPase activating protein 1 \|\| NM_013277 \|\| 12q13.12 |
| 74 | Thyroid hormone receptor interactor 13 \|\| NM_004237 \|\| 5p15.33 |
| 75 | Ubiquitin-conjugating enzyme E2T (putative) \|\| BF690859 \|\| 1q32.1 |
| 76 | Calpain 13 \|\| BX647678 \|\| |
| 77 | Peroxisomal acyl-CoA thioesterase 2B \|\| AK055797 \|\| 14q24.3 |
| 78 | Protein regulator of cytokinesis 1 \|\| NM_003981 \|\| 15q26.1 |
| 79 | SAM pointed domain containing ets transcription factor \|\| BC021299 \|\| 6p21.3 |
| 80 | Maba1 \|\| AB037745 \|\| 1p13.3 |
| 81 | Secreted frizzled-related protein 1 \|\| BC036503 \|\| 8p12-p11.1 |
| 82 | Tetraspanin 13 \|\| AK128509 \|\| 7p21.1 |
| 83 | MAD2 mitotic arrest deficient-like 1 (yeast) \|\| BQ215664 \|\| 4q27 |
| 84 | NIMA (never in mitosis gene a)-related kinase 2 \|\| BC043502 \|\| 1q32.2-q41 |
| 85 | Neural proliferation, differentiation and control, 1 \|\| AK054950 \|\| 9q34.3 |
| 86 | G-protein signalling modulator 2 (AGS3-like, C. elegans) \|\| NM_013295 \|\| 1p13.3 |
| 87 | Discs, large homolog 7 (Drosophila) \|\| NM_014750 \|\| 14q22.3 |
| 88 | Solute carrier family 40 (iron-regulated transporter), member 1 \|\| NM_014585 \|\| 2q32 |
| 89 | Origin recognition complex, subunit 6 homolog-like (yeast) \|\| NM_014321 \|\| 16q12 |
| 90 | Exonuclease 1 \|\| NM_130398 \|\| 1q42-q43 |
| 91 | Kinesin family member 20A \|\| AK025790 \|\| 5q31 |
| 92 | Epsin 3 \|\| AK000785 \|\| 17q21.33 |
| 93 | Pituitary tumor-transforming 1 \|\| BQ278502 \|\| 5q35.1 |
| 94 | RAS-like, estrogen-regulated, growth inhibitor \|\| BC007997 \|\| 12p12.3 |
| 95 | Transmembrane protein 25 \|\| AK124814 \|\| 11q23.3 |
| 96 | Phosphoglycerate dehydrogenase \|\| AK093306 \|\| 1p12 |
| 97 | Solute carrier family 9 (sodium/hydrogen exchanger), isoform 3 regulator 1 \|\| BX648303 \|\| 17q25 |
| 98 | Family with sequence similarity 64, member A \|\| CR590914 \|\| 17p13.2 |
| 99 | Sema domain, Immunoglobulin domain (Ig), short basic domain, secreted, (semaphorin) 3C \|\| NI |
| 100 | Cell division cycle associated 1 \|\| NM_145697 \|\| 1q23.3 |
| 101 | Breast carcinoma amplified sequence 1 \|\| CR749643 \|\| 20q13.2-q13.3 |
| 102 | G protein-coupled receptor, family C, group 5, member C \|\| AK131210 \|\| 17q25 |
| 103 | RNA-binding protein \|\| BC071585 \|\| 4p13-p12 |
| 104 | Cold inducible RNA binding protein \|\| AK095781 \|\| 19p13.3 |
| 105 | Asp (abnormal spindle)-like, microcephaly associated (Drosophila) \|\| AY367065 \|\| 1q31 |
| 106 | Spindle pole body component 25 homolog (S. cerevisiae) \|\| BC022255 \|\| 2q24.3 |
| 107 | Nuclear receptor interacting protein 1 \|\| NM_003489 \|\| 21q11.2 |
| 108 | Adenylate kinase 5 \|\| NM_012093 \|\| 1p31 |
| 109 | Kinesin family member C1 \|\| XM_371813 \|\| 6p21.3 |
| 110 | Apolipoprotein B, mRNA editing enzyme, catalytic polypeptide-like 3B \|\| AK024854 \|\| 22q13.1-q1 [symbol] |
| 111 | Ring finger protein 103 \|\| NM_005667 \|\| 2p11.2 |
| 112 | Cyclin G2 \|\| AK092638 \|\| 4q21.1 |
| 113 | Low density lipoprotein receptor-related protein 8, apolipoprotein e receptor \|\| NM_004631 \|\| 1p3 |
| 114 | Phenylethanolamine N-methyltransferase \|\| NM_002686 \|\| 17q21-q22 |
| 115 | Frizzled homolog 9 (Drosophila) \|\| BC026333 \|\| 7q11.23 |
| 116 | TPX2, microtubule-associated protein homolog (Xenopus laevis) \|\| NM_012112 \|\| 20q11.2 |
| 117 | Similar to common salivary protein 1 \|\| BU558247 \|\| 16p13.3 |
| 118 | Serine-arginine repressor protein (35 kDa) \|\| AK027365 \|\| 6q15 |
| 119 | Prominin 1 \|\| AF117225 \|\| 4p15.32 |
| 120 | Leucine-rich repeats and immunoglobulin-like domains 1 \|\| BC071561 \|\| 3p14 |
| 121 | Zinc finger protein 552 \|\| AK023769 \|\| 19q13.43 |
| 122 | Tumor protein p53 inducible nuclear protein 1 \|\| AK125880 \|\| 8q22 |
| 123 | Chloride intracellular channel 6 \|\| AF448439 \|\| 21q22.12 |
| 124 | 4-aminobutyrate aminotransferase \|\| NM_020686 \|\| 16p13.2 |
| 125 | Cytochrome c oxidase subunit VIc \|\| AK128382 \|\| 8q22-q23 |
| 126 | Potassium channel tetramerisation domain containing 3 \|\| NM_016121\|\| 1q41 |
| 127 | Potassium intermediate/small conductance calcium-activated channel, subfamily N, member 4 \|\| |
| 128 | Quinoid dihydropteridine reductase \|\| AK124952 \|\| 4p15.31 |
| 129 | SRY (sex determining region Y)-box 11 \|\| AB028641 \|\| 2p25 |
| 130 | Angiogenin, ribonuclease, RNase A family, 5 \|\| NM_194430 \|\| 14q11.1 |
| 131 | Likely ortholog of mouse dilute suppressor \|\| BC082990 \|\| 2q35 |
| 132 | CTP synthase \|\| BC009408 \|\| 1p34.1 |
| 133 | Enhancer of zeste homolog 2 (Drosophila) \|\| AK023816 \|\| 7q35-q36 |
| 134 | Solute carrier family 27 (fatty acid transporter), member 2 \|\| AK223145 \|\| 15q21.2 |
| 135 | Complement component 4B \|\| BC063289 \|\| 6p21.3 |
| 136 | Hedgehog acyltransferase \|\| AK128135 \|\| 1q32 |
| 137 | Desmocollin 2 \|\| NM_004949 \|\| 18q12.1 |
| 138 | Neuropeptide Y receptor Y1 \|\| AB209237 \|\| 4q31.3-q32 |
| 139 | Chromosome 3 open reading frame 18 \|\| AK127002 \|\| 3p21.3 |
| 140 | Forkhead box M1 \|\| NM_202002 \|\| 12p13 |
| 141 | Sperm associated antigen 5 \|\| NM_006461 \|\| 17q11.2 |
| 142 | T-box 3 (ulnar mammary syndrome) \|\| NM_016569 \|\| 12q24.1 |
| 143 | Karyopherin alpha 2 (RAG cohort 1, importin alpha 1) \|\| BC067848 \|\| 17q23.1-q23.3 |
| 144 | Ropporin, rhophilin associated protein 1 \|\| AL133624 \|\| 3q21.1 |
| 145 | RAD51 homolog (RecA homolog, E. coli) (S. cerevisiae) \|\| AL833420 \|\| 15q15.1 |
| 146 | Uncharacterized bone marrow protein BM039 \|\| AK023669 \|\| 16q23.2 |
| 147 | Superoxide dismutase 2, mitochondrial \|\| AK097395 \|\| 6q25.3 |
| 148 | Melanoma antigen family D, 2 \|\| AK092463 \|\| Xp11.2 |
| 149 | Titin-cap (telethonin) \|\| AK096328 \|\| 17q12 |
| 150 | Cyclin B1 \|\| NM_031966 \|\| 5q12 |
| 151 | Similar to ovostatin 2 \|\| XM_495907 \|\| 12p13.31 |
| 152 | Protein kinase, X-linked \|\| NM_005044 \|\| Xp22.3 |
| 153 | Transcription factor AP-2 beta (activating enhancer binding protein 2 beta) \|\| NM_003221 \|\| 6p21 |
| 154 | Nuclease sensitive element binding protein 1 \|\| BF525416 \|\| 1p34 |
| 155 | Kinesin family member 13B \|\| NM_015254 \|\| 8p12 |
| 156 | Hypothetical protein MGC4251 \|\| BM542308 \|\| 17q21.31 |
| 157 | PDZ binding kinase \|\| NM_018492 \|\| 8p21.2 |
| 158 | Hypothetical protein FLJ10901 \|\| AK001763 \|\| 1q32.1 |
| 159 | Heat shock 70 kDa protein 5 (glucose-regulated protein, 78 kDa) binding protein 1 \|\| NM_017870\| |
| 160 | Transforming growth factor, beta 3 \|\| AK122902 \|\| 14q24 |
| 161 | Glutathione S-transferase M3 (brain) \|\| NM_000849 \|\| 1p13.3 |
| 162 | Progesterone receptor \|\| NM_000926 \|\| 11q22-q23 |
| 163 | Phospholipase C, gamma 2 (phosphatidylinositol-specific) \|\| AB208914 \|\| 16q24.1 |
| 164 | Inositol polyphosphate-4-phosphatase, type II, 105 kDa \|\| BX649090 \|\| 4q31.21 |
| 165 | Kinesin family member 23 \|\| NM_138555\|\| 15q23 |
| 166 | GTP binding protein 4 \|\| NM_012341 \|\| 10p15-p14 |
| 167 | Stathmin 1/oncoprotein 18 \|\| BX647885 \|\| 1p36.1-p35 |
| 168 | Trefoil factor 1 (breast cancer, estrogen-inducible sequence expressed in) \|\| BM923753 \|\| 21q22. |
| 169 | Hematological and neurological expressed 1 \|\| BC039343 \|\| 17q25.1 |
| 170 | Stanniocalcin 2 \|\| NM_003714 \|\| 5q35.1 |
| 171 | DEP domain containing 1 \|\| BC065304 \|\| 1p31.2 |
| 172 | Bloom syndrome \|\| BC034480 \|\| 15q26.1 |
| 173 | Thymidine kinase 1, soluble \|\| BF683703 \|\| 17q23.2-q25.3 |
| 174 | Pleckstrin homology domain containing, family F (with FYVE domain) member 2 \|\| NM_024613\| |
| 175 | MKL/myocardin-like 2 \|\| NM_014048 \|\| 16p13.12 |
| 176 | Cell division cycle 2, G1 to S and G2 to M \|\| CR933728 \|\| 10q21.1 |
| 177 | Pituitary tumor-transforming 2 \|\| AF095288 \|\| 4p12 |
| 178 | Epoxide hydrolase 2, cytoplasmic \|\| NM_001979 \|\| 8p21-p12 |
| 179 | RAB27B, member RAS oncogene family \|\| AF131784 \|\| 18q21.2 |
| 180 | Insulin-like growth factor binding protein 4 \|\| NM_001552 \|\| 17q12-q21.1 |
| 181 | Tweety homolog 1 (Drosophila) \|\| AK126690 \|\| 19q13.4 |
| 182 | B/K protein \|\| NM_016524 \|\| 16p12.3 |
| 183 | Epithelial protein lost in neoplasm beta \|\| BX647194 \|\| 12q13 |
| 184 | DNA replication factor \|\| AB053172 \|\| 16q24.3 |
| 185 | Proteasome (prosome, macropain) 26S subunit, non-ATPase, 3 \|\| D67025 \|\| 17q21.1 |
| 186 | Dual specificity phosphatase 4 \|\| NM_057158 \|\| 8p12-p11 |
| 187 | Hypothetical protein FLJ21127 \|\| AK128417 \|\| 12q24.11 |
| 188 | Acyl-Coenzyme A oxidase 2, branched chain \|\| BC033517 \|\| 3p14.3 |
| 189 | Hypothetical protein DKFZp434L142 \|\| NM_016613 \|\| 4q32.1 |
| 190 | H2A histone family, member Z \|\| AK056803 \|\| 4q24 |
| 191 | Transcription elongation factor A (SII)-like 1 \|\| BM690957 \|\| Xq22.1 |
| 192 | ADP-ribosylation factor-like 6 interacting protein 2 \|\| AK026946 \|\| 2p22.2-p22.1 |
| 193 | Solute carrier family 1 (neuronal/epithelial high affinity glutamate transporter, system Xag), mem |
| 194 | Phytanoyl-CoA dioxygenase domain containing 1 \|\| AK095000 \|\| 9q34.11 |
| 195 | SFRS protein kinase 1 \|\| AJ318054 \|\| 6p21.3-p21.2 |
| 196 | Lunatic fringe homolog (Drosophila) \|\| AK096284 \|\| 7p22 |
| 197 | SID1 transmembrane family, member 1 \|\| AK000181 \|\| 3q13.2 |
| 198 | Inositol 1,4,5-triphosphate receptor, type 1 \|\| D26070 \|\| 3p26-p25 |
| 199 | Dachshund homolog 1 (Drosophila) \|\| NM_080759 \|\| 13q22 |
| 200 | Asparagine synthetase \|\| NM_133436 \|\| 7q21.3 |
| 201 | Amphiregulin (schwannoma-derived growth factor) \|\| AK023449 \|\| 4q13-q21 |
| 202 | Retinoic acid induced 2 \|\| BC027937 \|\| Xp22 |
| 203 | Smith-Magenis syndrome chromosome region, candidate 6 \|\| AB209609 \|\| 17p11.2 |
| 204 | Nuclear factor (erythroid-derived 2)-like 3 \|\| NM_004289 \|\| 7p15-p14 |
| 205 | MCM6 minichromosome maintenance deficient 6 (MIS5 homolog, S. pombe) (S. cerevisiae) \|\| N |
| 206 | Ectonucleotide pyrophosphatase/phosphodiesterase 1 \|\| NM_006208 \|\| 6q22-q23 |
| 207 | Dynein, cytoplasmic, light polypeptide 2B \|\| BC035232 \|\| 16q23.3 |
| 208 | TAL1 (SCL) interrupting locus \|\| NM_003035 \|\| 1q32 |
| 209 | Insulin receptor substrate 1 \|\| NM_005544 \|\| 2q36 |
| 210 | SET binding protein 1 \|\| BX640904 \|\| 18q21.1 |
| 211 | Chromosome 6 open reading frame 211 \|\| AK022972 \|\| 6q25.1 |
| 212 | Chromosome 15 open reading frame 23 \|\| CR602848 \|\| 15q15.1 |
| 213 | Chitinase 3-like 2 \|\| U58515 \|\| 1p13.3 |
| 214 | MLF1 interacting protein \|\| AF516710 \|\| 4q35.1 |
| 215 | Crystallin, alpha B \|\| BU734674 \|\| 11q22.3-q23.1 |
| 216 | Kinesin family member 11 \|\| NM_004523 \|\| 10q24.1 |
| 217 | Mal, T-cell differentiation protein 2 \|\| AY007723 \|\| |
| 218 | Hypothetical protein CG003 \|\| U50534 \|\| 13q13.1 |
| 219 | Fucosyltransferase 8 (alpha (1,6) fucosyltransferase) \|\| AJ536056 \|\| 14q24.3 |
| 220 | DC13 protein \|\| AK123983 \|\| 16q23.2 |
| 221 | Pericentriolar material 1 \|\| NM_006197 \|\| 6p22-p21.3 |
| 222 | Tetraspanin 1 \|\| BQ216899 \|\| 1p34.1 |
| 223 | Phosphoserine aminotransferase 1 \|\| NM_058179 \|\| 9q21.2 |
| 224 | Cytochrome b-5 \|\| AB209617 \|\| 18q23 |
| 225 | Family with sequence similarity 54, member A \|\| AK125758 \|\| 6q23.3 |
| 226 | Thymidylate synthetase \|\| BQ056428 \|\| 18p11.32 |
| 227 | Hypothetical protein MGC11242 \|\| BC002865 \|\| 17q21.32 |
| 228 | High-mobility group box 3 \|\| BX537505 \|\| Xq28 |
| 229 | Uracil-DNA glycosylase 2 \|\| BC004877 \|\| 5p15.2-p13.1 |
| 230 | Peroxisomal D3,D2-enoyl-CoA isomerase \|\| AB209917 \|\| 6p24.3 |
| 231 | Cadherin 3, type 1, P-cadherin (placental) \|\| BC041846 \|\| 16q22.1 |
| 232 | V-yes-1 Yamaguchi sarcoma viral related oncogene homolog \|\| BC059394 \|\| 8q13 |
| 233 | Cadherin 1, type 1, E-cadherin (epithelial) \|\| NM_004360 \|\| 16q22.1 |
| 234 | Chromosome 2 open reading frame 23 \|\| AK023172 \|\| 2p11.2 |
| 235 | Mucin 1, transmembrane \|\| J05581 \|\| 1q21 |
| 236 | BTG family, member 3 \|\| BU730087 \|\| 21q21.1-q21.2 |
| 237 | Keratin 8 \|\| CR607281 \|\| 12q13 |
| 238 | Lysosomal-associated membrane protein 3 \|\| BC032940 \|\| 3q26.3-q27 |
| 239 | Kelch/ankyrin repeat containing cyclin A1 interacting protein \|\| BC032482 \|\| 1q23.3 |
| 240 | V-myb myeloblastosis viral oncogene homolog (avian)-like 1 \|\| XM_034274 \|\| 8q22 |
| 241 | Protein kinase (cAMP-dependent, catalytic) inhibitor beta \|\| CR749456 \|\| 6q22.31 |
| 242 | Nucleolar and spindle associated protein 1 \|\| AK222819 \|\| 15q15.1 |
| 243 | Galactosidase, alpha \|\| NM_000169 \|\| Xq22 |
| 244 | Tumor protein p53 binding protein, 2 \|\| NM_005426 \|\| 1q42.1 |
| 245 | Transcription factor CP2-like 2 \|\| BC063299 \|\| 2p25.1 |
| 246 | SPARC related modular calcium binding 2 \|\| AL832303 \|\| 6q27 |
| 247 | Inhibitor of DNA binding 4, dominant negative helix-loop-helix protein \|\| BM701438 \|\| 6p22-p21 |
| 248 | Tripartite motif-containing 2 \|\| AF220018 \|\| 4q31.3 |
| 249 | Kallikrein 6 (neurosin, zyme) \|\| NM_002774 \|\| 19q13.3 |
| 250 | Early growth response 3 \|\| NM_004430 \|\| 8p23-p21 |
| 251 | ATPase family, AAA domain containing 2 \|\| CR749832 \|\| 8q24.13 |
| 252 | Family with sequence similarity 63, member A \|\| AB037811 \|\| 1q21.2 |
| 253 | Keratin 16 (focal non-epidermolytic palmoplantar keratoderma) \|\| BC039169 \|\| 17q12-q21 |
| 254 | Phosphoribosyl transferase domain containing 1 \|\| NM_020200 \|\| 10p12.1 |
| 255 | Metallothionein-like 5, testis-specific (tesmin) \|\| AK128308 \|\| 11q13.2-q13.3 |
| 256 | Dual specificity phosphatase 6 \|\| BC037236 \|\| 12q22-q23 |
| 257 | BTG family, member 2 \|\| NM_006763 \|\| 1q32 |
| 258 | Acyl-Coenzyme A dehydrogenase, short/branched chain \|\| NM_001609 \|\| 10q25-q26 |
| 259 | Midline 1 (Opitz/BBB syndrome) \|\| AF041210 \|\| Xp22 |
| 260 | NAD(P)H dehydrogenase, quinone 1 \|\| NM_000903 \|\| 16q22.1 |
| 261 | Harakiri, BCL2 interacting protein (contains only BH3 domain) \|\| D83699 \|\| 12q24.22 |
| 262 | V-set domain containing T cell activation inhibitor 1 \|\| BX648021 \|\| 1p13.1 |
| 263 | Keratin 23 (histone deacetylase inducible) \|\| NM_015515 \|\| 17q21.2 |
| 264 | Squalene epoxidase \|\| NM_003129 \|\| 8q24.1 |
| 265 | Aldehyde dehydrogenase 6 family, member A1 \|\| NM_005589 \|\| 14q24.3 |
| 266 | Glutathione S-transferase pi \|\| BM926728 \|\| 11q13 |
| 267 | Gasdermin-like \|\| BX647700 \|\| 17q21.1 |
| 268 | Claudin 4 \|\| AK126462 \|\| 7q11.23 |
| 269 | Fc fragment of IgG binding protein \|\| NM_003890 \|\| 19q13.1 |
| 270 | Myosin VI \|\| NM_004999 \|\| 6q13 |
| 271 | Moesin \|\| NM_002444 \|\| Xq11.2-q12 |
| 272 | Cyclin A2 \|\| CR604810 \|\| 4q25-q31 |
| 273 | Huntingtin interacting protein K \|\| AF370428 \|\| 15q15.3 |
| 274 | Pro-oncosis receptor inducing membrane injury gene \|\| AK075420 \|\| 11q22.1 |
| 275 | Cysteine-rich motor neuron 1 \|\| AF167706 \|\| 2p21 |
| 276 | Calcium/calmodulin-dependent protein kinase II \|\| CR604926 \|\| 1p36.12 |
| 277 | START domain containing 10 \|\| AB209473 \|\| 11q13 |
| 278 | Tensin like C1 domain containing phosphatase \|\| CR936725 \|\| 12q13.13 |
| 279 | Inositol(myo)-1(or 4)-monophosphatase 2 \|\| BM924855 \|\| 18p11.2 |
| 280 | 7-dehydrocholesterol reductase \|\| BC000054 \|\| 11q13.2-q13.5 |
| 281 | Uridine-cytidine kinase 2 \|\| BX640859 \|\| 1q23 |
| 282 | Plasminogen activator, tissue \|\| BX641021 \|\| 8p12 |
| 283 | Cytochrome P450, family 4, subfamily X, polypeptide 1 \|\| NM_178033 \|\| 1p33 |
| 284 | Chromosome 10 open reading frame 116 \|\| AL157440 \|\| 10q23.2 |
| 285 | Period homolog 2 (Drosophila) \|\| NM_022817 \|\| 2q37.3 |
| 286 | CAMP responsive element binding protein 3-like 4 \|\| AY049977 \|\| 1q21.3 |
| 287 | F-box and leucine-rich repeat protein 3 \|\| AL833187 \|\| 13q22 |
| 288 | Ubiquitin specific protease 1 \|\| BC050525 \|\| 1p32.1-p31.3 |
| 289 | Amphoterin induced gene 2 \|\| NM_181847 \|\| 12q13.11 |
| 290 | Frizzled homolog 7 (Drosophila) \|\| AB017365 \|\| 2q33 |
| 291 | Chemokine-like factor super family 7 \|\| AK055554 \|\| 3p23 |
| 292 | Macrophage receptor with collagenous structure \|\| BC016004 \|\| 2q12-q13 |
| 293 | FYVE and coiled-coil domain containing 1 \|\| AJ292348 \|\| 3p21.31 |
| 294 | UDP-glucose dehydrogenase \|\| AF061016 \|\| 4p15.1 |
| 295 | FN5 protein \|\| AK098204 \|\| 11q13.3-q23.3 |
| 296 | Biliverdin reductase A \|\| BX647539 \|\| 7p14-cen |
| 297 | Zinc finger protein 462 \|\| NM_021224 \|\| 9q31.2 |
| 298 | Fatty acid binding protein 5 (psoriasis-associated) \|\| BG282526 \|\| 8q21.13 |
| 299 | CDP-diacylglycerol synthase (phosphatidate cytidylyltransferase) 1 \|\| NM_001263 \|\| 4q21.23 |
| 300 | UDP-N-acetyl-alpha-D-galactosamine:polypeptide N-acetylgalactosaminyltransferase 6 (GalNAc |
| 301 | Parathyroid hormone-like hormone \|\| J03580 \|\| 12p12.1-p11.2 |
| 302 | Replication factor C (activator 1) 4, 37 kDa \|\| NM_002916 \|\| 3q27 |
| 303 | CHK1 checkpoint homolog (S. pombe) \|\| NM_001274 \|\| 11q24-q24 |
| 304 | Growth differentiation factor 15 \|\| BQ883534 \|\| 19p13.1-13.2 |
| 305 | Receptor-interacting serine-threonine kinase 2 \|\| AY358814 \|\| 8q21 |
| 306 | Hypothetical protein FLJ20366 \|\| AB195679 \|\| 8q23.2 |
| 307 | Synaptotagmin-like 2 \|\| AY386362 \|\| 11q14 |
| 308 | Guanldinoacetate N-methyltransferase \|\| BM541904 \|\| 19p13.3 |
| 309 | GLI-Kruppel family member GLI3 (Greig cephalopolysyndactyly syndrome) \|\| M57609 \|\| 7p13 |
| 310 | Thymosin-like 8 \|\| BG471140 \|\| Xq21.33-q22.3 |
| 311 | CDC28 protein kinase regulatory subunit 1B \|\| BQ278454 \|\| 1q21.2 |
| 312 | Ubiquitin protein ligase E3 component n-recognin 1 \|\| NM_174916 \|\| 15q13 |
| 313 | DnaJ (Hsp40) homolog, subfamily A, member 4 \|\| NM_018602 \|\| 15q25.1 |
| 314 | Signal transducer and activator of transcription 3 interacting protein 1 \|\| AK095780 \|\| 18q12.2 |
| 315 | Diaphanous homolog 3 (Drosophila) \|\| BC034952 \|\| 13q21.2 |
| 316 | SUMO-1 activating enzyme subunit 2 \|\| AK124730 \|\| 19q12 |
| 317 | Collagen, type XIV, alpha 1 (undulin) \|\| NM_021110 \|\| 8q23 |
| 318 | Islet cell autoantigen 1, 69 kDa \|\| CR605198 \|\| 7p22 |
| 319 | Heat shock 27 kDa protein 1 \|\| BM541936 \|\| 7q11.23 |
| 320 | Peroxisomal biogenesis factor 11A \|\| AL360141 \|\| 15q26.1 |
| 321 | DEAD (Asp-Glu-Ala-Asp) box polypeptide 39 \|\| CR592759 \|\| 19p13.12 |
| 322 | Programmed cell death 4 (neoplastic transformation inhibitor) \|\| BX537500 \|\| 10q24 |
| 323 | Cyclin-dependent kinase inhibitor 2A (melanoma, p16, inhibits CDK4) \|\| BM719878 \|\| 9p21 |
| 324 | HRAS-like suppressor 2 \|\| AK025029 \|\| 11q12.3 |
| 325 | Interleukin enhancer binding factor 2, 45 kDa \|\| BG121872 \|\| 1q21.3 |
| 326 | Serine (or cysteine) proteinase inhibitor, clade B (ovalbumin), member 5 \|\| BX640597 \|\| 18q21.3 |
| 327 | BCL2-associated athanogene \|\| BM799512 \|\| 9p12 |
| 328 | Carboxypeptidase E \|\| NM_001873 \|\| 4q32.3 |
| 329 | CDC28 protein kinase regulatory subunit 2 \|\| BQ898943 \|\| 9q22 |
| 330 | Cbp/p300-interacting transactivator, with Glu/Asp-rich carboxy-terminal domain, 1 \|\| BM664781 [symbol] |
| 331 | Chromosome 1 open reading frame 38 \|\| AK094833 \|\| 1p35.3 |
| 332 | Contactin associated protein-like 2 \|\| NM_014141 \|\| 7q35-q36 |
| 333 | Chitinase 3-like 1 (cartilage glycoprotein-39) \|\| AB209459 \|\| 1q32.1 |
| 334 | SH3 domain binding glutamic acid-rich protein like \|\| AK024892 \|\| Xq13.3 |
| 335 | NCK adaptor protein 1 \|\| NM_006153 \|\| 3q21 |
| 336 | Cytochrome b reductase 1 \|\| AL136693 \|\| 2q31.1 |
| 337 | Tripartite motif-containing 29 \|\| BX648072 \|\| 11q22-q23 |
| 338 | Solute carrier family 25 (mitochondrial deoxynucleotide carrier), member 19 \|\| AK097882 \|\| 17q2 [symbol] |
| 339 | Chromatin assembly factor 1 , subunit B (p60) \|\| NM_005441 \|\| 21q22.13 |
| 340 | Aldo-keto reductase family 7, member A3 (aflatoxin aldehyde reductase) \|\| NM_012067 \|\| 1p35.1 |
| 341 | Hypothetical protein FLJ20186 \|\| NM_207514 \|\| 16q24.3 |
| 342 | CDK2-associated protein 1 \|\| NM_004642 \|\| 12q24.31 |
| 343 | Homogentisate 1,2-dioxygenase (homogentisate oxidase) \|\| BC071757 \|\| 3q21-q23 |
| 344 | Glycine receptor, beta \|\| NM_00824 \|\| 4q31.3 |
| 345 | Pleckstrin and Sec7 domain containing 3 \|\| NM_015310 \|\| 8pter-p23.3 |
| 346 | Interferon, gamma-inducible protein 30 \|\| AK123477 \|\| 19p13.1 |
| 347 | Annexin A9 \|\| AJ009985 \|\| 1q21 |
| 348 | Proteasome (prosome, macropain) subunit, alpha type, 7 \|\| AK127210 \|\| 20q13.33 |
| 349 | RAB17, member RAS oncogene family \|\| BX647412 \|\| 2q37.3 |
| 350 | KIAA0310 \|\| XM_088459 \|\| 9q34.3 |
| 351 | CDC7 cell division cycle 7 (S. cerevisiae) \|\| AB209337 \|\| 1p22 |
| 352 | Chromosome 7 open reading frame 24 \|\| BF570959 \|\| 7p15-p14 |
| 353 | CD164 antigen, siaiomucin \|\| BC040317 \|\| 6q21 |
| 354 | Chromosome 22 open reading frame 18 \|\| AK123479 \|\| 22q13.2 |
| 355 | PDZ domain containing 1 \|\| NM_002614 \|\| 1q21 |
| 356 | Collagen, type IX, alpha 3 \|\| NM_001853 \|\| 20q13.3 |
| 357 | KIAA0101 \|\| AY358648 \|\| 15q22.31 |
| 358 | Sal-like 2 (Drosophila) \|\| NM_005407 \|\| 14q11.1-q12 |
| 359 | Mitogen-activated protein kinase kinase kinase 1 \|\| XM_042066 \|\| 5q11.2 |
| 360 | Neurobeachin \|\| NM_015678 \|\| 13q13 |
| 361 | Chromosome 14 open reading frame 132 \|\| BC043593 \|\| 14q32.2 |
| 362 | SRY (sex determining region Y)-box 10 \|\| BC018808 \|\| 22q13.1 |
| 363 | NDRG family member 2 \|\| AK096999 \|\| 14q11.2 |
| 364 | PiggyBac transposable element derived 5 \|\| NM_024554 \|\| 1q42.13 |
| 365 | Mago-nashi homolog \|\| NM_018048 \|\| 12p13.2 |
| 366 | Slit homolog 2 (Drosophila) \|\| AF055585 \|\| 4p15.2 |
| 367 | Cyclin-dependent kinase inhibitor 3 (CDK2-associated dual specificity phosphatase) \|\| BQ05633 [symbol] |
| 368 | ZW10 interactor \|\| NM_001005414 \|\| 10q21-q22 |
| 369 | Nei endonuclease VIII-like 1 (E. coli) \|\| AK128372 \|\| 15q23 |
| 370 | SLIT and NTRK-like family, member 6 \|\| NM_032229 \|\| 13q31.1 |
| 371 | Centromere protein F, 350/400ka (mitosin) \|\| NM_016343 \|\| 1q32-q41 |
| 372 | Glycyl-tRNA synthetase \|\| NM_002047 \|\| 7p15 |
| 373 | Pentraxin-related gene, rapidly induced by IL-1 beta \|\| NM_002852 \|\| 3q25 |
| 374 | Timeless homolog (Drosophila) \|\| BC050557 \|\| 12q12-q13 |
| 375 | Kinesin family member 4A \|\| AF071592 \|\| Xq13.1 |
| 376 | Cyclin E1 \|\| BC035498 \|\| 19q12 |
| 377 | CGI-119 protein \|\| AK127285 \|\| 12q14.1-q15 |
| 378 | Flap structure-specific endonuclease 1 \|\| NM_004111 \|\| 11q12 |
| 379 | Fatty acid binding protein 7, brain \|\| AB208815 \|\| 6q22-q23 |
| 380 | Gene model 83 \|\| AK001693 \|\| 8q22.3 |
| 381 | D component of complement (adipsin) \|\| BQ712715 \|\| 19p13.3 |
| 382 | Isocitrate dehydrogenase 2 (NADP+), mitochondrial \|\| AK127371 \|\| 15q26.1 |
| 383 | DEK oncogene (DNA binding) \|\| BX641063 \|\| 6p23 |
| 384 | Leucine zipper transcription factor-like 1 \|\| BC042483 \|\| 3p21.3 |
| 385 | Prion protein (p27-30) (Creutzfeld-Jakob disease, Gerstmann-Strausler-Scheinker syndrome, fa [symbol] |
| 386 | Histamine N-methyltransferase \|\| NM_006895 \|\| 2q22.1 |
| 387 | Tumor protein D52 \|\| NM_005079 \|\| 8q21 |
| 388 | CNKSR family member 3 \|\| AY328891 \|\| 6q25.2 |
| 389 | Interferon-related developmental regulator 1 \|\| NM_001007245 \|\| 7q22-q31 |
| 390 | Fibromodulin \|\| BC035281 \|\| 1q32 |
| 391 | Cell division cycle associated 3 \|\| AK092246 \|\| 12p13 |
| 392 | Wolfram syndrome 1 (wolframin) \|\| BC069213 \|\| 4p16 |
| 393 | Glutamic pyruvate transaminase (alanine aminotransferase) 2 \|\| NM_133443 \|\| 16q12.1 |
| 394 | N-acylsphingosine amidohydrolase (acid ceramidase) 1 \|\| NM_004315 \|\| 8p22-p21.3 |
| 395 | Cytoplasmic polyadenylation element binding protein 3 \|\| NM_014912 \|\| 10q23.32 |
| 396 | GDNF family receptor alpha 1 \|\| AF038421 \|\| 10q26 |
| 397 | Extra spindle poles like 1 (S. cerevisiae) \|\| D79987 \|\| 12q |
| 398 | Hypothetical protein FLJ10706 \|\| AK127098 \|\| 1q24.2 |
| 399 | Interferon, alpha-inducible protein (clone IFI-15K) \|\| BQ279256 \|\| 1p36.33 |
| 400 | Fucosidase, alpha-L-1, tissue \|\| BC017338 \|\| 1p34 |
| 401 | CDC6 cell division cycle 6 homolog (S. cerevisiae) \|\| NM_001254 \|\| 17q21.3 |
| 402 | Gamma-glutamyl hydrolase (conjugase, folylpolygammaglutamyl hydrolase) \|\| CD359152 \|\| 8q12 |
| 403 | Lactate dehydrogenase B \|\| AB209231 \|\| 12p12.2-p12.1 |
| 404 | Cytokine inducible SH2-containing protein \|\| NM_013324 \|\| 3p21.3 |
| 405 | Sema domain, immunoglobulin domain (Ig), short basic domain, secreted, (semaphorin) 3E \|\| NI |
| 406 | Insulin-like growth factor 1 (somatomedin C) \|\| NM_000618 \|\| 12q22-q23 |
| 407 | Leucine rich repeat containing 17 \|\| NM_005824 \|\| 7q22.1 |
| 408 | Transforming, acidic coiled-coil containing protein 3 \|\| AJ243997 \|\| 4p16.3 |
| 409 | Prefoldin 5 \|\| AK024094 \|\| 12q12 |
| 410 | Proline-rich nuclear receptor coactivator 2 \|\| BC085018 \|\| 1p36.11 |
| 411 | Biotinidase \|\| NM_000060 \|\| 3p25 |
| 412 | RAD51 associated protein 1 \|\| CR625391 \|\| 12p13.2-p13.1 |
| 413 | Sema domain, immunoglobulin domain (Ig), short basic domain, secreted, (semaphorin) 3B \|\| A [symbol] |
| 414 | Rho GTPase activating protein 21 \|\| AF480466 \|\| 10p12.1 |
| 415 | Trinucleotide repeat containing 9 \|\| AK095095 \|\| 16q12.1 |
| 416 | RAD21 homolog (S. pombe) \|\| NM_006265 \|\| 8q24 |
| 417 | Hypothetical protein FLJ20605 \|\| AK125512 \|\| 1q41 |
| 418 | Methylenetetrahydrofolate dehydrogenase (NADP+ dependent) 1-like \|\| AK127089 \|\| 6q25.1 |
| 419 | MutS homolog 2, colon cancer, nonpolyposis type 1 (E. coli) \|\| AK223284 \|\| 2p22-p21 |
| 420 | Transient receptor potential cation channel, subfamily V, member 2 \|\| AK126996 \|\| 17p11.2 |
| 421 | Chromosome 20 open reading frame 24 \|\| BG482041 \|\| 20q11.23 |
| 422 | RAB11A, member RAS oncogene family \|\| BC013348 \|\| 15q21.3-q22.31 |
| 423 | Histone deacetylase 2 \|\| AB209190 \|\| 6q21 |
| 424 | Coronin, actin binding protein, 1C \|\| NM_014325 \|\| 12q24.1 |
| 425 | Iroquois homeobox protein 5 \|\| AY335945 \|\| 16q11.2-q13 |
| 426 | Small nuclear ribonucleoprotein polypeptide A′ \|\| AK090986 \|\| 15q26.3 |
| 427 | Epithelial membrane protein 2 \|\| AK096403 \|\| 16p13.2 |
| 428 | Protein tyrosine phosphatase type IVA, member 2 \|\| NM_003479 \|\| 1p35 |
| 429 | MCM7 minichromosome maintenance deficient 7 (S. cerevisiae) \|\| NM_182776 \|\| 7q21.3-q22.1 |
| 430 | RAB11 family interacting protein 1 (class I) \|\| NM_001002814 \|\| 8p11.22 |
| 431 | V-rel reticuloendotheliosis viral oncogene homolog B, nuclear factor of kappa light polypeptide g [symbol] |
| 432 | SHC SH2-domain binding protein 1 \|\| NM_024745 \|\| 16q11.2 |
| 433 | Transglutaminase 1 (K polypeptide epidermal type I, protein-glutamine-gamma-glutamyltransfer [symbol] |
| 434 | Nuclear factor I/A \|\| BX648791 \|\| 1p31.3-p31.2 |
| 435 | ATP-binding cassette, sub-family C (CFTR/MRP), member 8 \|\| NM_000352 \|\| 11p15.1 |
| 436 | Frizzled homolog 4 (Drosophila) \|\| AB032417 \|\| 11q14.2 |
| 437 | ADP-ribosylation factor like 7 \|\| NM_005737 \|\| 2q37.1 |
| 438 | THO complex 4 \|\| BQ279142 \|\| 17q25.3 |
| 439 | Coiled-coil-helix-coiled-coil-helix domain containing 5 \|\| AK024631 \|\| 2q13 |
| 440 | Chromosome 14 open reading frame 100 \|\| AK128628 \|\| 14q23.1 |
| 441 | Lamin B1 \|\| BC052951 \|\| 5q23.3-q31.1 |
| 442 | CTD (carboxy-terminal domain, RNA polymerase II, polypeptide A) small phosphatase 1 \|\| AF22 |
| 443 | Mitochondrial ribosomal protein L15 \|\| BQ278804 \|\| 8q11.2-q13 |
| 444 | Cryptochrome 2 (photolyase-like) \|\| BC035161 \|\| 11p11.2 |
| 445 | Membrane cofactor protein (CD46, trophoblast-lymphocyte cross-reactive antigen) \|\| BX537451 |
| 446 | Trophinin associated protein (tastin) \|\| AK128056 \|\| 12q13.12 |
| 447 | G protein-coupled receptor associated sorting protein 1 \|\| NM_014710 \|\| Xq22.1 |
| 448 | Kallikrein 10 \|\| AK026045 \|\| 19q13.3-q13.4 |
| 449 | Purinergic receptor P2Y, G-protein coupled, 5 \|\| BC045651 \|\| 13q14 |
| 450 | Serine (or cysteine) proteinase inhibitor, clade A (alpha-1 antiproteinase, antitrypsin), member 3 |
| 451 | Flavin containing monooxygenase 5 \|\| NM_001461 \|\| 1q21.1 |
| 452 | F-box and leucine-rich repeat protein 5 \|\| NM_033535 \|\| 4p15.33 |
| 453 | Mitochondrial ribosomal protein S30 \|\| BX538300 \|\| 5q11 |
| 454 | MCM2 minichromosome maintenance deficient 2, mitotin (S. cerevisiae) \|\| NM_004526 \|\| 3q21 |
| 455 | Serine (or cysteine) proteinase inhibitor, clade A (alpha-1 antiproteinase, antitrypsin), member 5 |
| 456 | UNC-112 related protein 2 \|\| BC004347 \|\| 11q13.1 |
| 457 | Carnitine acetyltransferase \|\| NM_000755 \|\| 9q34.1 |
| 458 | Guanylate binding protein 5 \|\| AK090479 \|\| 1p22.2 |
| 459 | Chromosome 20 open reading frame 28 \|\| NM_015417 \|\| 20pter-q11.23 |
| 460 | Cyclin D-type binding-protein 1 \|\| CR614852 \|\| 15q14-q15 |
| 461 | Glioma tumor suppressor candidate region gene 2 \|\| AK024486 \|\| 19q13.3 |
| 462 | V-kit Hardy-Zuckerman 4 feline sarcoma viral oncogene homolog \|\| BC071593 \|\| 4q11-q12 |
| 463 | Chromosome condensation-related SMC-associated protein 1 \|\| D63880 \|\| 12p13.3 |
| 464 | Heterogeneous nuclear ribonucleoprotein H2 (H′) \|\| CR624721 \|\| Xq22 |
| 465 | Guanine monphosphate synthetase \|\| NM_003875 \|\| 3q24 |
| 466 | KIAA0182 protein \|\| NM_014615 \|\| 16q24.1 |
| 467 | DNA replication complex GINS protein PSF2 \|\| AK091519 \|\| 16q24.1 |
| 468 | Praja 2, RING-H2 motif containing \|\| NM_014819 \|\| 5q21.3 |
| 469 | Eukaryotic translation initiation factor 2C, 2 \|\| BC054491 \|\| 8q24 |
| 470 | Dehydrogenase/reductase (SDR family) member 2 \|\| AB209653 \|\| 14q11.2 |
| 471 | Hypothetical protein PRO1855 \|\| AK025328 \|\| 17q21.33 |
| 472 | Alpha-2-glycoprotein 1, zinc \|\| BC014470 \|\| 7q22.1 |
| 473 | Chromosome 20 open reading frame 23 \|\| AY166853 \|\| 20p11.23 |
| 474 | Zinc finger protein 339 \|\| AK022284 \|\| 20pter-q11.23 |
| 475 | Actin binding LIM protein family, member 3 \|\| AB020650 \|\| 5q32 |
| 476 | Hypothetical protein FLJ34633 \|\| AK091952 \|\| 1p36.11 |
| 477 | Potassium channel tetramerisation domain containing 9 \|\| AL117436 \|\| 8p21.1 |
| 478 | Hypothetical protein FLJ14490 \|\| AF370364 \|\| 1p34.2 |
| 479 | Keratin 5 (epidermolysis bullosa simplex, Dowling-Meara/Kobner/Weber-Cockayne types) \|\| M2 [symbol] |
| 480 | Homeo box B2 \|\| NM_002145 \|\| 17q21-q22 |
| 481 | Polymerase (DNA directed) iota \|\| BC032617 \|\| 18q21.1 |
| 482 | Microtubule associated serine/threonine kinase family member 4 \|\| XM_291141 \|\| 5q12.3 |
| 483 | Sin3-associated polypeptide, 30 kDa \|\| BC016757 \|\| 4q34.1 |
| 484 | N-myc (and STAT) interactor \|\| AK124323 \|\| 2p24.3-q21.3 |
| 485 | Claudin 23 \|\| BC016047 \|\| 8p23.1 |
| 486 | Solute carrier family 24 (sodium/potassium/calcium exchanger), member 1 \|\| AB014602 \|\| 15q22 |
| 487 | Thioredoxin interacting protein \|\| NM_006472 \|\| 1q21.1 |
| 488 | Solute carrier family 7 (cationic amino acid transporter, y+ system), member 2 \|\| NM_003046 \|\| B |
| 489 | Cyclin E2 \|\| NM_057749 \|\| 8q22.1 |
| 490 | Tumor protein, translationally-controlled 1 \|\| BG033621 \|\| 13q12-q14 |
| 491 | KIAA0141 \|\| NM_014773 \|\| 5q31.3 |
| 492 | Glycosyltransferase 8 domain containing 1 \|\| NM_001010983 \|\| 3p21.1 |
| 493 | Claudin 7 \|\| CR594337 \|\| 17p13 |
| 494 | NAD(P)H:quinone oxidoreductase type 3, polypeptide A2 \|\| AK123705 \|\| 1p36.13-q41 |
| 495 | Solute carrier family 16 (monocarboxylic acid transporters), member 3 \|\| AK127319 \|\| 17q25 |
| 496 | Calmegin \|\| AK093096 \|\| 4q28.3-q31.1 |
| 497 | DNA (cytosine-5-)-methyltransferase 3 beta \|\| NM_006892 \|\| 20q11.2 |
| 498 | Chromosome 9 open reading frame 58 \|\| AK128526 \|\| 9q34.13-q34.3 |
| 499 | Ovo-like 1(Drosophila) \|\| BC059408 \|\| 11q13 |
| 500 | Ubiquitin-conjugating enzyme E2S \|\| BM479313 \|\| 19q13.43 |
| 501 | BCL2-related protein A1 \|\| BF677029 \|\| 15q24.3 |
| 502 | Phospholipase A2, group VII (platelet-activating factor acetylhydrolase, plasma) \|\| BC025674 \|\| 6 |
| 503 | Myotubularin related protein 2 \|\| NM_201278 \|\| 11q22 |
| 504 | Guanylate binding protein 1, interferon-inducible, 67 kDa \|\| AB208912 \|\| 1p22.2 |
| 505 | MCM5 minichromosome maintenance deficient 5, cell division cycle 46 (S. cerevisiae) \|\| AB2096 |
| 506 | Mitochondrial tumor suppressor 1 \|\| NM_001001927 \|\| 8p22 |
| 507 | Thyroid hormone receptor associated protein 2 \|\| NM_015335 \|\| 12q24.21 |
| 508 | Vacuolar protein sorting 13C (yeast) \|\| AJ608771 \|\| 15q22.2 |
| 509 | Potassium channel, subfamily K, member 5 \|\| BC060793 \|\| 6p21 |
| 510 | LPS-responsive vesicle trafficking, beach and anchor containing \|\| AF467287 \|\| 4q31.23-q31.3 |
| 511 | Brain expressed, X-linked 1 \|\| BM804232 \|\| Xq21-q23 |
| 512 | Trichorhinophalangeal syndrome I \|\| NM_014112 \|\| 8q24.12 |
| 513 | Fibronectin leucine rich transmembrane protein 2 \|\| NM_013231 \|\| 14q24-q32 |
| 514 | Kinesin family member 15 \|\| NM_020242 \|\| 3p21.31 |
| 515 | Epithelial V-like antigen 1 \|\| NM_005797 \|\| 11q24 |
| 516 | Integrin, beta 2 (antigen CD18 (p95), lymphocyte function-associated antigen 1; macrophage ant |
| 517 | Integrin, beta 5 \|\| AK091595 \|\| 3q21.2 |
| 518 | Lipoma HMGIC fusion partner \|\| CR749848 \|\| 13q12 |
| 519 | Potassium channel tetramerisation domain containing 12 \|\| AF359381 \|\| 13q22.3 |
| 520 | Seven in absentia homolog 2 (Drosophila) \|\| NM_005067 \|\| 3q25 |
| 521 | Hematopoietic protein 1 \|\| NM_005337 \|\| 12q13.1 |
| 522 | V-fos FBJ murine osteosarcoma viral oncogene homolog \|\| BX647104 \|\| 14q24.3 |
| 523 | Cingulin-like 1 \|\| NM_032866 \|\| 15q21.3 |
| 524 | Chromosome 1 open reading frame 34 \|\| AB007921 \|\| 1p32.3 |
| 525 | RIO kinase 1 (yeast) \|\| BC006104 \|\| 6p24.3 |
| 526 | Hypothetical protein FLJ20425 \|\| AK000432 \|\| 4p16.3 |
| 527 | Junctional adhesion molecule 2 \|\| NM_021219 \|\| 21q21.2 |
| 528 | Bystin-like \|\| AK095253 \|\| 6p21.1 |
| 529 | TCDD-inducible poly(ADP-ribose) polymerase \|\| CR749647 \|\| 3q25.31 |
| 530 | Insulin-like growth factor binding protein 6 \|\| BM913156 \|\| 12q13 |
| 531 | Thrombospondin, type I, domain containing 1 \|\| AK096289 \|\| 13q14.3 |
| 532 | Ubiquinol-cytochrome c reductase hinge protein \|\| BF127835 \|\| 1p34.1 |
| 533 | Zinc finger and BTB domain containing 4 \|\| BC043352 \|\| 17p13.1 |
| 534 | Methylcrotonoyl-Coenzyme A carboxylase 2 (beta) \|\| AK094987 \|\| 5q12-q13 |
| 535 | Hypothetical protein FLJ20641 \|\| AK000648 \|\| 12q23.3 |
| 536 | ATP/GTP binding protein 1 \|\| AB028958 \|\| 9q21.33 |
| 537 | Rho GTPase-activating protein \|\| AL833062 \|\| 11q24-q25 |
| 538 | Kallikrein 8 (neuropsin/ovasin) \|\| BC040887 \|\| 19q13.3-q13.4 |
| 539 | Plrin (iron-binding nuclear protein) \|\| BX537579 \|\| Xp22.2 |
| 540 | Selenoprotein P, plasma, 1 \|\| BC030009 \|\| 5q31 |
| 541 | Caveolin 1, caveolae protein, 22 kDa \|\| NM_001753 \|\| 7q31.1 |
| 542 | Armadillo repeat containing, X-linked 1 \|\| AB039670 \|\| Xq21.33-q22.2 |
| 543 | Cold shock domain protein A \|\| AB209896 \|\| 12p13.1 |
| 544 | Nudix (nucleoside diphosphate linked moiety X)-type motif 6 \|\| AB209758 \|\| 4q26 |
| 545 | Forkhead box P1 \|\| BX538242 \|\| 3p14.1 |
| 546 | Zinc finger and BTB domain containing 16 \|\| AB208916 \|\| 11q23.1 |
| 547 | Sestrin 1 \|\| AK127043 \|\| 6q21 |
| 548 | Hypothetical protein FLJ21827 \|\| NM_020153 \|\| 11q23.3 |
| 549 | Comparative gene identification transcript 37 \|\| NM_016101 \|\| 16q22.1 |
| 550 | Mannosidase, alpha, class 2B, member 2 \|\| AB023152 \|\| 4p16.1 |
| 551 | Modulator of apoptosis 1 \|\| NM_022151 \|\| 14q32 |
| 552 | Peptidyl arginine deiminase, type II \|\| AB023211 \|\| 1p35.2-p35.1 |
| 553 | Protein tyrosine phosphatase, receptor type, T \|\| NM_133170 \|\| 20q12-q13 |
| 554 | Basic helix-loop-helix domain containing, class B, 2 \|\| NM_003670 \|\| 3p26 |
| 555 | Ubiquitin-like, containing PHD and RING finger domains, 1 \|\| AK074377 \|\| 19p13.3 |
| 556 | Chromosome 3 open reading frame 14 \|\| BM699794 \|\| 3p14.2 |
| 557 | Family with sequence similarity 38, member B \|\| AK056572 \|\| 18p11.22 |
| 558 | Guanine nucleotide binding protein (G protein), gamma 11 \|\| BF971151 \|\| 7q31-q32 |
| 559 | Hyaluronan and proteoglycan link protein 3 \|\| BC053689 \|\| 15q26.1 |
| 560 | Matrilin 3 \|\| NM_002381 \|\| 2p24-p23 |
| 561 | Proline-rich nuclear receptor coactivator 1 \|\| NM_006813 \|\| 6q15 |
| 562 | Phospholipid scramblase 4 \|\| BC028354 \|\| 3q24 |
| 563 | Hypothetical protein FLJ14966 \|\| AK027872 \|\| 11p15.3 |
| 564 | Topoisomerase (DNA) II alpha 170 kDa \|\| NM_001067 \|\| 17q21-q22 |
| 565 | Osteoglycin (osteoinductive factor, mimecan) \|\| NM_033014 \|\| 9q22 |
| 566 | Sperm protein SSPA11 \|\| AK125807 \|\| 17q21.33 |
| 567 | Primase, polypeptide 2A, 58 kDa \|\| NM_000947 \|\| 6p12-p11.1 |
| 568 | ATPase, Na+/K+ transporting, beta 3 polypeptide \|\| AK094673 \|\| 3q23 |
| 569 | ADP-ribosylation-like factor 5 interacting protein 5 \|\| NM_006407 \|\| 3p14 |
| 570 | NudC domain containing 1 \|\| BC043406 \|\| 8q23 |
| 571 | Tubulin tyrosine ligase-like family, member 4 \|\| D79995 \|\| 2p24.3-p24.1 |
| 572 | GREB1 protein \|\| NM_014668 \|\| 2p25.1 |
| 573 | Kelch domain containing 2 \|\| AK056298 \|\| 14q21.3 |
| 574 | Beta-transducin repeat containing \|\| NM_033637 \|\| 10q24.32 |
| 575 | Golgi autoantigen, golgin subfamily a, 5 \|\| NM_005113 \|\| 14q32.12-q32.13 |
| 576 | Rho guanine exchange factor (GEF) 16 \|\| CR609468 \|\| 1p36.3 |
| 577 | Coenzyme Q4 homolog (yeast) \|\| AK128853 \|\| 9q34.11 |
| 578 | Hypothetical gene supported by AF038182; BC009203 \|\| BC009203 \|\| 5q21.1 |
| 579 | IQ motif containing GTPase activating protein 2 \|\| NM_008633 \|\| 5q13.3 |
| 580 | Immunoglobulin (CD79A) binding protein 1 \|\| AK054596 \|\| Xq13.1-q13.3 |
| 581 | Cysteine and glycine-rich protein 2 \|\| AB209321 \|\| 12q21.1 |
| 582 | DIP13 beta \|\| BX649010 \|\| 12q24.1 |
| 583 | Solute carrier family 19 (thiamine transporter), member 2 \|\| AJ237724 \|\| 1q23.3 |
| 584 | Dynactin 4 (p62) \|\| AK125973 \|\| 5q31-q32 |
| 585 | Kallikrein 5 \|\| AY359010 \|\| 19q13.3-q13.4 |
| 586 | Chemokine (C—X—C motif) ligand 14 \|\| NM_004887 \|\| 5q31 |
| 587 | Golgi phosphoprotein 2 \|\| NM_016548 \|\| 9q21.33 |
| 588 | Trophoblast glycoprotein \|\| NM_006670 \|\| 6q14-q15 |
| 589 | Proliferating cell nuclear antigen \|\| BE896331 \|\| 20pter-p12 |
| 590 | ADAM-like, decysin 1 \|\| Y13323 \|\| 8p21.2 |
| 591 | Lymphocyte antigen 6 complex, locus D \|\| BC034542 \|\| 8q24-qter |
| 592 | Ras suppressor protein 1 \|\| NM_012425 \|\| 10p13 |
| 593 | Cingulin \|\| AF263462 \|\| 1q21 |
| 594 | Basic transcription factor 3 \|\| BX537826 \|\| 5q13.2 |
| 595 | Membrane targeting (tandem) C2 domain containing 1 \|\| NM_152332 \|\| 14q32.12 |
| 596 | MutS homolog 6 (E. coli) \|\| BC071594 \|\| 2p16 |
| 597 | Microtubule associated serine/threonine kinase-like \|\| AK123004 \|\| 10p12.1 |
| 598 | Jerky homolog-like (mouse) \|\| NM_003772 \|\| 11q21 |
| 599 | High mobility group nucleosomal binding domain 4 \|\| NM_005353 \|\| 6p21.3 |
| 600 | Polyribonucleotide nucleotidyltransferase 1 \|\| BC053660 \|\| 2p15 |
| 601 | Epidermal retinal dehydrogenase 2 \|\| NM_138969 \|\| 8q12.1 |
| 602 | Hypothetical protein FLJ23129 \|\| AK127011 \|\| 1p31.2 |
| 603 | Matrix metalloproteinase 9 (gelatinase B, 92 kDa gelatinase, 92 kDa type IV collagenase) \|\| BC00 [symbol] |
| 604 | Monoamine oxidase A \|\| NM_000240 \|\| Xp11.4-p11.3 |
| 605 | B-box and SPRY domain containing \|\| AK092607 \|\| 9q32 |
| 606 | Hydroxysteroid (17-beta) dehydrogenase 4 \|\| AB208932 \|\| 5q21 |
| 607 | Interleukin 13 receptor, alpha 1 \|\| Y10659 \|\| Xq24 |
| 608 | Ankyrin repeat, family A (RFXANK-like), 2 \|\| NM_023039 \|\| 5q12-q13 |
| 609 | Dual specificity phosphatase 5 \|\| NM_004419 \|\| 10q25 |
| 610 | Phosphofructokinase, platelet \|\| AK126153 \|\| 10p15.3-p15.2 |
| 611 | Integrin, beta-like 1 (with EGF-like repeat domains) \|\| AK095102 \|\| 13q33 |
| 612 | Cyclin-dependent kinase inhibitor 2D (p19, inhibits CDK4) \|\| NM_001800 \|\| 19p13 |
| 613 | S100 calcium binding protein A14 \|\| BG674026 \|\| 1q21.3 |
| 614 | Antigen identified by monoclonal antibody KI-67 \|\| NM_002417 \|\| 10q25-qter |
| 615 | Chromosome 6 open reading frame 66 \|\| CD555939 \|\| 6q16.1 |
| 616 | Transforming growth factor, beta receptor II (70/80 kDa) \|\| BX648313 \|\| 3p22 |
| 617 | G-2 and S-phase expressed 1 \|\| NM_016426 \|\| 22q13.2-q13.3 |
| 618 | Sortilin related receptor, L(DLR class) A repeats-containing \|\| NM_003105 \|\| 11q23.2-q24.2 |
| 619 | Notch homolog 1, translocation-associated (Drosophila) \|\| NM_017617 \|\| 9q34.3 |
| 620 | Lymphocyte cytosolic protein 1 (L-plastin) \|\| NM_002298 \|\| 13q14.3 |
| 621 | Cystatin C (amyloid angiopathy and cerebral hemorrhage) \|\| BX647523 \|\| 20p11.21 |
| 622 | Indoleamine-pyrrole 2,3 dioxygenase \|\| M34455 \|\| 8p12-p11 |
| 623 | SET and MYND, domain containing 2 \|\| BC049367 \|\| 1q41 |
| 624 | Tumor necrosis factor (ligand) superfamily, member 13b \|\| NM_006573 \|\| 13q32-34 |
| 625 | Hypothetical protein LOC257106 \|\| BX537846 \|\| 1q23.3 |
| 626 | Protein kinase D3 \|\| NM_005813 \|\| 2p21 |
| 627 | Cell division cycle 25C \|\| BC039100 \|\| 5q31 |
| 628 | Neuroblastoma RAS viral (v-ras) oncogene homolog \|\| X02751 \|\| 1p13.2 |
| 629 | CD24 antigen (small cell lung carcinoma cluster 4 antigen) \|\| AK125531 \|\| 6q21 |
| 630 | Down syndrome critical region gene 2 \|\| CR624273 \|\| 21q22.3 |
| 631 | Monogenic, audiogenic seizure susceptibility 1 homolog (mouse) \|\| AF435925 \|\| 5q13 |
| 632 | Solute carrier family 9 (sodium/hydrogen exchanger), isoform 6 \|\| BC035029 \|\| Xq26.3 |
| 633 | Phosphatidylinositol glycan, class H \|\| BC071849 \|\| 14q11-q24 |
| 634 | Block of proliferation 1 \|\| NM_015201 \|\| 8q24.3 |
| 635 | CGI-146 protein \|\| NM_016076 \|\| 1q44 |
| 636 | Galactosylceramidase (Krabbe disease) \|\| NM_000153 \|\| 14q31 |
| 637 | Integral membrane protein 2A \|\| AB209310 \|\| Xq13.3-Xq21.2 |
| 638 | FSH primary response (LRPR1 homolog, rat) 1 \|\| NM_006733 \|\| Xq22.1 |
| 639 | Protease, serine; 23 \|\| AL832007 \|\| 11q14.1 |
| 640 | Transducer of ERBB2, 1 \|\| BC031406 \|\| 17q21 |
| 641 | Transforming growth factor, beta receptor III (betaglycan, 300 kDa) \|\| L07594 \|\| 1p33-p32 |
| 642 | Hyaluronan-mediated motility receptor (RHAMM) \|\| AF032862 \|\| 5q33.2-qter |
| 643 | S100 calcium binding protein A8 (calgranulin A) \|\| BG739729 \|\| 1q21 |
| 644 | Tetratricopeptide repeat domain 13 \|\| NM_024525 \|\| 1q42.2 |
| 645 | ORM1-like 2 (S. cerevisiae) \|\| CR621685 \|\| 12q13.2 |
| 646 | Runt-related transcription factor 1; translocated to, 1 (cyclin D-related) \|\| NM_004349 \|\| 8q22 |
| 647 | Thyroid hormone receptor associated protein 4 \|\| NM_014815 \|\| 17q21.1 |
| 648 | Alanyl-tRNA synthetase \|\| AK222824 \|\| 16q22 |
| 649 | Integral membrane protein 2B \|\| BX537657 \|\| 13q14.3 |
| 650 | Hepatoma-derived growth factor (high-mobility group protein 1-like) \|\| NM_004494 \|\| 1q21-q23 |
| 651 | SRY (sex determining region Y)-box 9 (campomelic dysplasia, autosomal sex-reversal) \|\| NM_0 [symbol] |
| 652 | Sprouty homolog 2 (Drosophila) \|\| BX648582 \|\| 13q31.1 |
| 653 | Polo-like kinase 4 (Drosophila) \|\| NM_014264 \|\| 4q27-q28 |
| 654 | Regulator of G-protein signalling 5 \|\| NM_003617 \|\| 1q23.1 |
| 655 | Rho-related BTB domain containing 2 \|\| AB018260 \|\| 8p21.3 |
| 656 | Ets variant gene 6 (TEL oncogene) \|\| NM_001987 \|\| 12p13 |
| 657 | Secretoglobin, family 1D, member 2 \|\| AW015055 \|\| 11q13 |
| 658 | Butyrate-induced transcript 1 \|\| BX648759 \|\| 15q22.2 |
| 659 | Testis expressed sequence 10 \|\| AK000294 \|\| 9q31.1 |
| 660 | DnaJ (Hsp40) homolog, subfamily C, member 1 \|\| CR613772 \|\| 10p12.31 |
| 661 | Protein tyrosine phosphatase-like (proline instead of catalytic arginine), member a \|\| AY455942 \|\| |
| 662 | Synaptotagmin-like 4 (granuphilin-a) \|\| AL832596 \|\| |
| 663 | Chromosome 16 open reading frame 45 \|\| AK092923 \|\| 16p13.11 |
| 664 | Tyrosine aminotransferase \|\| NM_000353 \|\| 16q22.1 |
| 665 | Proline-serine-threonine phosphatase interacting protein 1 \|\| CR593209 \|\| 15q24-q25.1 |
| 666 | Nuclear factor I/B \|\| BX537698 \|\| 9p24.1 |
| 667 | 3-hydroxymethyl-3-methylglutaryl-Coenzyme A lyase (hydroxymethylglutaricaciduria) \|\| BG0335 [symbol] |
| 668 | SNF related kinase \|\| CR749621 \|\| 3p22.1 |
| 669 | RAN binding protein 1 \|\| AK094410 \|\| 22q11.21 |
| 670 | Chromosome 10 open reading frame 18 \|\| XM_374765 \|\| 10p15.1 |
| 671 | Activated leukocyte cell adhesion molecule \|\| AL833702 \|\| 3q13.1 |
| 672 | TP53 activated protein 1 \|\| BC068535 \|\| 7q21.1 |
| 673 | Tubulin, beta 6 \|\| AK092677 \|\| 18p11.21 |
| 674 | Potassium voltage-gated channel, delayed-rectifier, subfamily S, member 3 \|\| BC015947 \|\| 2p24 |
| 675 | Transmembrane protease, serine 5 (spinesin) \|\| AF495727 \|\| 11q |
| 676 | Zinc finger, FYVE domain containing 21 \|\| AK055909 \|\| 14q32.33 |
| 677 | Methylenetetrahydrofolate dehydrogenase (NADP+ dependent) 2, methenyltetrahydrofolate cyclc [symbol] |
| 678 | CK2 interacting protein 1; HQ0024c protein \|\| AK125609 \|\| 1q21.2 |
| 679 | Hypothetical protein FLJ13639 \|\| AK023701 \|\| 13q14.3 |
| 680 | A disintegrin and metalloproteinase domain 8 \|\| NM_001109 \|\| 10q26.3 |
| 681 | Calcium modulating ligand \|\| NM_001745 \|\| 5q23 |
| 682 | Chromosome 1 open reading frame 21 \|\| NM_030806 \|\| 1q25 |
| 683 | Trans-prenyltransferase \|\| AB209763 \|\| 10p12.1 |
| 684 | Lysosomal associated protein transmembrane 4 beta \|\| BC038117 \|\| 8q22.1 |
| 685 | Solute carrier family 2 (facilitated glucose/fructose transporter), member 5 \|\| BC035878 \|\| 1p36.2 |
| 686 | N-myristoyltransferase 2 \|\| NM_004808 \|\| 10p13 |
| 687 | HRAS-like suppressor \|\| BC005856 \|\| 3q29 |
| 688 | Aurora kinase B \|\| CD049340 \|\| 17p13.1 |
| 689 | Mitochondrial solute carrier protein \|\| AK127666 \|\| 8p21.2 |
| 690 | ORM1-like 3 (S. cerevisiae) \|\| AK093063 \|\| 17q12-q21.1 |
| 691 | F-box protein 5 \|\| AK055221 \|\| 6q25-q26 |
| 692 | Ubiquitin specific protease 13 (isopeptidase T-3) \|\| BC049199 \|\| 3q26.2-q26.3 |
| 693 | Interleukin 32 \|\| BF569086 \|\| 16p13.3 |
| 694 | CREBBP/EP300 inhibitor 1 \|\| NM_014335 \|\| 15q21.1-q21.2 |
| 695 | Cellular retinoic acid binding protein 1 \|\| AK096006 \|\| 15q24 |
| 696 | Chromatin modifying protein 4C \|\| NM_152284 \|\| 8q21.13 |
| 697 | Nucleoporin 93 kDa \|\| AK056637 \|\| 16q13 |
| 698 | Leucine zipper protein 5 \|\| BX537845 \|\| 7q36.3 |
| 699 | Cell division cycle 25B \|\| NM_021874 \|\| 20p13 |
| 700 | Hyperparathyroidism 2 (with jaw tumor) \|\| NM_024529 \|\| 1q25 |
| 701 | KIAA0020 \|\| AL832245 \|\| 9p24.2 |
| 702 | Eukaryotic translation elongation factor 1 alpha 2 \|\| AB209064 \|\| 20q13.3 |
| 703 | RAD17 homolog (S. pombe) \|\| AF076838 \|\| 5q13 |
| 704 | Pyruvate dehydrogenase (lipoamide) alpha 1 \|\| BG036317 \|\| Xp22.2-p22.1 |
| 705 | All-trans-13,14-dihydroretinol saturase \|\| BC068517 \|\| 2p11.2 |
| 706 | Chromatin assembly factor 1, subunit A (p150) \|\| NM_005483 \|\| 19p13.3 |
| 707 | RAB11 family interacting protein 3 (class II) \|\| BC051360 \|\| 16p13.3 |
| 708 | Morf4 family associated protein 1-like 1 \|\| AF258591 \|\| 4p16.1 |
| 709 | Peroxisomal long-chain acyl-coA thioesterase \|\| NM_006821 \|\| 14q24.3 |
| 710 | KIAA0514 \|\| NM_014696 \|\| 10q11.22 |
| 711 | Tripartite motif-containing 45 \|\| AY669488 \|\| 1p13.1 |
| 712 | Proteasome (prosome, macropain) activator subunit 4 \|\| NM_014614 \|\| 2p16.3 |
| 713 | Myosin X \|\| AB018342 \|\| 5p15.1-p14.3 |
| 714 | Keratin 19 \|\| BG292068 \|\| 17q21.2 |
| 715 | HSPC163 protein \|\| BX649076 \|\| 1q42.12 |
| 716 | ATPase, Na+/K+ transporting, alpha 2 (+) polypeptide \|\| NM_000702 \|\| 1q21-q23 |
| 717 | Lysosomal associated multispanning membrane protein 5 \|\| CR607037 \|\| 1p34 |
| 718 | Zinc finger, DHHC domain containing 13 \|\| BC036020 \|\| 11p15.1 |
| 719 | Programmed cell death 10 \|\| BC002506 \|\| 3q26.1 |
| 720 | High-mobility group box 2 \|\| CR600021 \|\| 4q31 |
| 721 | Claudin 3 \|\| BM701226 \|\| 7q11.23 |
| 722 | Ectonucleotide pyrophosphatase/phosphodiesterase 5 (putative function) \|\| BX647968 \|\| 6p21.1-1 |
| 723 | Peroxisomal biogenesis factor 7 \|\| BC031606 \|\| 6q21-q22.2 |
| 724 | SEC15-like 1 (S. cerevisiae) \|\| AK128190 \|\| 10q23.33 |
| 725 | Peroxisomal biogenesis factor 11 gamma \|\| AK127684 \|\| 19p13.2 |
| 726 | Hypothetical protein FLJ20485 \|\| NM_019042 \|\| 7q22.3 |
| 727 | Mitochondrial carrier homolog 2 (C. elegans) \|\| AY380792 \|\| 11p11.2 |
| 728 | Peroxisome biogenesis factor 13 \|\| AK093866 \|\| 2p14-p16 |
| 729 | Relaxin 2 \|\| NM_005059 \|\| 9p24.1 |
| 730 | Leukocyte immunoglobulin-like receptor, subfamily B (with TM and ITIM domains), member 6 \|\| [symbol] |
| 731 | Transporter 1, ATP-binding cassette, sub-family B (MDR/TAP) \|\| BX648013 \|\| 6p21.3 |
| 732 | Junctional adhesion molecule 3 \|\| NM_032801 \|\| 11q25 |
| 733 | Chromosome 6 open reading frame 96 \|\| AK000634 \|\| 6q25.1 |
| 734 | Monoglyceride lipase \|\| NM_007283 \|\| 3q21.3 |
| 735 | Early growth response 1 \|\| NM_001964 \|\| 5q31.1 |
| 736 | Phospholipid scramblase 1 \|\| AB006746 \|\| 3q23 |
| 737 | Kelch-like 7 (Drosophila) \|\| NM_018845 \|\| 7p15.3 |
| 738 | CGI-49 protein \|\| NM_016002 \|\| 1q44 |
| 739 | Laminin, alpha 3 \|\| NM_198129 \|\| 18q11.2 |
| 740 | Enhancer of yellow 2 homolog (Drosophila) \|\| AK095651 \|\| 8q23.1 |
| 741 | Fatty acid amide hydrolase \|\| NM_001441 \|\| 1p35-p34 |
| 742 | Carbohydrate (chondroitin 4) sulfotransferase 11 \|\| AL833176 \|\| 12q |
| 743 | Serine protease inhibitor, Kunitz type, 2 \|\| AK127479 \|\| 19q13.1 |
| 744 | ATP synthase, H+ transporting, mitochondrial F1 complex, gamma polypeptide 1 \|\| BF131627 \|\| |
| 745 | SEC14-like 2 (S. cerevisiae) \|\| AB033012 \|\| 22q12.2 |
| 746 | MCM10 minichromosome maintenance deficient 10 (S. cerevisiae) \|\| AL136840 \|\| 10p13 |
| 747 | Keratin 15 \|\| AK122864 \|\| 17q21.2 |
| 748 | Syndecan 1 \|\| NM_001006946 \|\| 2p24.1 |
| 749 | ATP-binding cassette, sub-family C (CFTR/MRP), member 3 \|\| NM_020038 \|\| 17q22 |
| 750 | Apolipoprotein C-I \|\| AJ249921 \|\| 19q13.2 |
| 751 | Stress-induced-phosphoprotein 1 (Hsp70/Hsp90-organizing protein) \|\| BC039299 \|\| 11q13 |
| 752 | Sodium channel, nonvoltage-gated 1 alpha \|\| AK172792 \|\| 12p13 |
| 753 | N-myc downstream regulated gene 1 \|\| AK124709 \|\| 8q24.3 |
| 754 | Zinc finger protein 281 \|\| BC060820 \|\| 1q32.1 |
| 755 | Cdc42 guanine nucleotide exchange factor (GEF) 9 \|\| NM_015185 \|\| Xq11.2 |
| 756 | Cardiomyopathy associated 5 \|\| NM_153610 \|\| 5q14.1 |
| 757 | TEA domain family member 4 \|\| NM_003213 \|\| 12p13.2-p13.3 |
| 758 | Rho GTPase activating protein 22 \|\| BC047096 \|\| 10q11.22 |
| 759 | Brain-specific anglogenesis inhibitor 2 \|\| NM_001703 \|\| 1p35 |
| 760 | Calcium activated nucleotidase 1 \|\| NM_138793 \|\| 17q25.3 |
| 761 | VAMP (vesicle-associated membrane protein)-associated protein A, 33 kDa \|\| NM_003574 \|\| 1Bp |
| 762 | Adipose differentiation-related protein \|\| NM_001122 \|\| 9p22.1 |
| 763 | Glutamyl-prolyl-tRNA synthetase \|\| NM_004446 \|\| 1q41-q42 |
| 764 | KIAA1598 \|\| AK091578 \|\| 10q25.3 |
| 765 | Plakophilin 3 \|\| NM_007183 \|\| 11p15 |
| 766 | Hypothetical protein DKFZp761P0423 \|\| XM_291277 \|\| 8p23.1 |
| 767 | Tetratricopeptide repeat domain 10 \|\| AK126668 \|\| 13q12.1 |
| 768 | Similar to RIKEN cDNA 5730528L13 gene \|\| AK092292 \|\| 9q31.1 |
| 769 | UMP-CMP kinase \|\| AK025258 \|\| |
| 770 | Adaptor protein containing pH domain, PTB domain and leucine zipper motif 1 \|\| NM_012096 \|\| 3 |
| 771 | Gelsolin (amyloidosis, Finnish type) \|\| AK125819 \|\| 9q33 |
| 772 | Ribosomal protein L5 \|\| AK095815 \|\| 1p22.1 |
| 773 | WD repeat domain 5 \|\| NM_017588 \|\| 9q34 |
| 774 | Liver-specific bHLH-Zip transcription factor \|\| AK126834 \|\| 19q13.12 |
| 775 | Hypothetical protein MGC13183 \|\| AK027638 \|\| 12p13.33 |
| 776 | Mitochondrial ribosomal protein L27 \|\| NM_148571 \|\| 17q21.3-q22 |
| 777 | Msh homeo box homolog 2 (Drosophila) \|\| D89377 \|\| 5q34-q35 |
| 778 | OCIA domain containing 1 \|\| AK123529 \|\| 4p11 |
| 779 | Paralemmin \|\| NM_002579 \|\| 19p13.3 |
| 780 | LETM1 domain containing 1 \|\| AK123080 \|\| 12q13.12 |
| 781 | Chaperonin containing TCP1, subunit 5 (epsilon) \|\| NM_012073 \|\| 5p15.2 |
| 782 | ATPase, H+ transporting, lysosomal accessory protein 1 \|\| AK090462 \|\| Xq28 |
| 783 | Tumor necorsis factor, alpha-induced protein 3 \|\| BC041790 \|\| 6q23 |
| 784 | UDP glycosyltransferase 2 family, polypeptide B11 \|\| AK124272 \|\| 4q13.2 |
| 785 | Granzyme B (granzyme 2, cyctotoxic T-lymphocyte-associated serine esterase 1) \|\| BQ052893 \|\| |
| 786 | Putative NFkB activating protein 373 \|\| BX647545 \|\| 1p31.2 |
| 787 | Cirrhosis, autosomal recessive 1A (cirhin) \|\| AB075868 \|\| 16q22.1 |
| 788 | Cadherin, EGF LAG seven-pass G-type receptor 2 (flamingo homolog, Drosophila) \|\| AF234887 |
| 789 | Chromosome 20 open reading frame 35 \|\| AL390160 \|\| 20q13.12 |
| 790 | E2F transcription factor 5, p130-binding \|\| AB209185 \|\| 8q21.2 |
| 791 | Myxovirus (influenza virus) resistance 2 (mouse) \|\| AK122952 \|\| 21q22.3 |
| 792 | Insulin-like growth factor 2 (somatomedin A) \|\| AK074614 \|\| 11p15.5 |
| 793 | Prefoldin 2 \|\| BF203500 \|\| 1q23.3 |
| 794 | FRAS1 related extracellular matrix 1 \|\| BX648240 \|\| 9p22.3 |
| 795 | Zinc finger protein 521 \|\| AK027354 \|\| 18q11.2 |
| 796 | Hypothetical protein MGC7036 \|\| AK054942 \|\| 12q24.31 |
| 797 | Epithelial stromal interaction 1 (breast) \|\| AL831953 \|\| 13q13.3 |
| 798 | 6-pyruvoyltetrahydropterin synthase \|\| BG249563 \|\| 11q22.3-q23.3 |
| 799 | Centromere protein E, 312 kDa \|\| NM_001813 \|\| 4q24-q25 |
| 800 | Histone 1, H2ak \|\| BC034487 \|\| 6p22-p21.3 |
| 801 | Methylcrotonoyl-Coenzyme A carboxylase 1 (alpha) \|\| BC042453 \|\| 3q27 |
| 802 | Polo-like kinase 2 (Drosophila) \|\| AF059617 \|\| 5q12.1-q13.2 |
| 803 | Runt-related transcription factor 3 \|\| NM_004350 \|\| 1p36 |
| 804 | Jumonji domain containing 2B \|\| AB020683 \|\| 19p13.3 |
| 805 | Chromosome 20 open reading frame 172 \|\| BC026011 \|\| 20q11.23 |
| 806 | V-myc myelocytomatosis viral oncogene homolog (avian) \|\| NM_002467 \|\| 8q24.12-q24.13 |
| 807 | Transcription elongation factor B (SIII), polypeptide 1 (15 kDa, elongin C) \|\| AK057889 \|\| 8q21.11 |
| 808 | Hypothetical protein FLJ10647 \|\| BM911450 \|\| 1p34.3 |
| 809 | Histone deacetylase 11 \|\| AL834223 \|\| 3p25.1 |
| 810 | Chemokine (C-X3-C motif) ligand 1 \|\| AB209037 \|\| 16q13 |
| 811 | Tyrosine 3-monooxygenase/tryptophan-5-monooxygenase activation protein, zeta polypeptide \|\| |
| 812 | Brain protein J3 \|\| BU589543 \|\| 7q21.3 |
| 813 | Erythrocyte membrane protein band 4.1 like 5 \|\| BC054508 \|\| 2q14.2 |
| 814 | Src-like-adaptor \|\| BX647569 \|\| 8q22.3-qter |
| 815 | V-erb-b2 erythroblastic leukemia viral oncogene homolog 3 (avian) \|\| NM_001982 \|\| 12q13 |
| 816 | Nuclear factor of kappa light polypeptide gene enhancer in B-cells inhibitor, epsilon \|\| BC063609 |
| 817 | TAR (HIV) RNA binding protein 1 \|\| U38847 \|\| 1q42.3 |
| 818 | Dicer1, Dcr-1 homolog (Drosophila) \|\| NM_177438 \|\| 14q32.13 |
| 819 | Lymphocyte-specific protein 1 \|\| AK056576 \|\| 11p15.5 |
| 820 | DnaJ (Hsp40) homolog, subfamily B, member 11 \|\| BC046500 \|\| 3q27.3 |
| 821 | Dyskeratosis congenita 1, dyskerin \|\| BC009928 \|\| Xq28 |
| 822 | Rhotekin \|\| NM_033046 \|\| 2p13.1 |
| 823 | Mst3 and SOK1-related kinase \|\| BC070056 \|\| Xq26.2 |
| 824 | Dmx-like 1 \|\| AJ005821 \|\| 5q22 |
| 825 | S-phase kinase-associated protein 1A (p19A) \|\| NM_006930 \|\| 5q31 |
| 826 | Myoglobin \|\| BF670653 \|\| 22q13.1 |
| 827 | Similar to RIKEN cDNA 4921524J17 \|\| BX647945 \|\| 16q11.2 |
| 828 | Hypothetical protein FLJ21019 \|\| AB208939 \|\| 17q21.2 |
| 829 | NCK-associated protein 1 \|\| AB011159 \|\| 2q32 |
| 830 | Mitochondrial ribosomal protein L13 \|\| AK123239 \|\| 8q22.1-q22.3 |
| 831 | Heat shock 22 kDa protein 8 \|\| NM_014365 \|\| 12q24.23 |
| 832 | Neuromedin B \|\| BE781314 \|\| 15q22-qter |
| 833 | Polycomb group ring finger 4 \|\| NM_005180 \|\| 10p11.23 |
| 834 | SRY (sex determining region Y)-box 13 \|\| NM_005686 \|\| 1q32 |
| 835 | Hypothetical protein LOC339745 \|\| BC071613 \|\| 2q22.1 |
| 836 | N-acetylglucosamine-1-phosphotransferase, gamma subunit \|\| AK126110 \|\| 16p13.3 |
| 837 | Zinc finger, MYND domain containing 10 \|\| AB209621 \|\| 3p21.3 |
| 838 | Cytochrome b-561 domain containing 2 \|\| BX641103 \|\| 3p21.3 |
| 839 | O-acetyltransferase \|\| BC063284 \|\| 7q21.3 |
| 840 | CD52 antigen (CAMPATH-1 antigen) \|\| BU739882 \|\| 1p36 |
| 841 | Protein tyrosine phosphatase, non-receptor type 14 \|\| NM_005401 \|\| 1q32.2 |
| 842 | Fc fragment of IgG, receptor, transporter, alpha \|\| AK074734 \|\| 19q13.3 |
| 843 | Chromosome 12 open reading frame 11 \|\| BC003081 \|\| 12p11.23 |
| 844 | Dehydrogenase/reductase (SDR family) member 8 \|\| AY358553 \|\| 4q22.1 |
| 845 | Chromosome 8 open reading frame 4 \|\| CR600720 \|\| 8p11.2 |
| 846 | Cytoskeleton associated protein 2 \|\| NM_018204 \|\| 13q14 |
| 847 | E-1 enzyme \|\| AF113125 \|\| 4q21.3 |
| 848 | Hypothetical protein FLJ12078 \|\| BX538123 \|\| 5q15 |
| 849 | Cathepsin F \|\| BC013359 \|\| 11q13 |
| 850 | Serum deprivation response (phosphatidylserine binding protein) \|\| NM_004657 \|\| 2q32-q33 |
| 851 | Baculoviral IAP repeat-containing 3 \|\| NM_001165 \|\| 11q22 |
| 852 | Hypothetical protein FLJ20607 \|\| BQ935360 \|\| 12q24.22 |
| 853 | Solute carrier family 15, member 3 \|\| AK127216 \|\| 11q12.2 |
| 854 | Keratin 17 \|\| BX647923 \|\| 17q12-q21 |
| 855 | Ubiquitin-like 3 \|\| BC044582 \|\| 13q12-q13 |
| 856 | Ubiquitin protein ligase E3A (human papilloma virus E6-associated protein, Angelman syndrom [symbol] |
| 857 | Gila maturation factor, gamma \|\| BG259135 \|\| 19q13.2 |
| 858 | Elongation of very long chain fatty acids (FEN1/Elo2, SUR4/Elo3, yeast)-like 2 \|\| BC050278 \|\| 6p [symbol] |
| 859 | KIAA0590 gene product \|\| AB209020 \|\| 16p13.3 |
| 860 | Chromosome 6 open reading frame 80 \|\| AK092592 \|\| 6q23.1-q24.1 |
| 861 | Chromosome 10 open reading frame 7 \|\| AK023925 \|\| 10p13 |
| 862 | Inositol 1,3,4-triphosphate 5/6 kinase \|\| AK024887 \|\| 14q31 |
| 863 | Myosin regulatory light chain interacting protein \|\| NM_013262 \|\| 6q23-p22.3 |
| 864 | Hepsin (transmembrane protease, serine 1) \|\| AK125670 \|\| 19q11-q13.2 |
| 865 | Ribosomal protein S4, X-linked \|\| BM994563 \|\| Xq13.1 |
| 866 | Lymphotoxin beta (TNF superfamily, member 3) \|\| AK095821 \|\| 6p21.3 |
| 867 | Zinc finger protein 395 \|\| NM_018660 \|\| 8p21.1 |
| 868 | EPS8-like 1 \|\| AF370395 \|\| 19q13.42 |
| 869 | SH2 domain protein 2A \|\| NM_003975 \|\| 1q21 |
| 870 | Nuclear receptor subfamily 3, group C, member 1 (glucocorticoid receptor) \|\| NM_000176 \|\| 5q3 [symbol] |
| 871 | Glyceraldehyde-3-phosphate dehydrogenase \|\| BF983396 \|\| 12p13 |
| 872 | Transmembrane 7 superfamily member 2 \|\| AF023676 \|\| 11q13 |
| 873 | Proteoglycan 1, secretory granule \|\| CD359027 \|\| 10q22.1 |
| 874 | Hypothetical protein FLJ20171 \|\| BX647570 \|\| 8q22.1 |
| 875 | Chromosome 1 open reading frame 31 \|\| CR602593 \|\| 1q42.2 |
| 876 | Hypothetical protein MGC2603 \|\| AK024326 \|\| 1p36.11 |
| 877 | Chemokine (C—X—C motif) ligand 12 (stromal cell-derived factor 1) \|\| BX647204 \|\| 10q11.1 |
| 878 | A disintegrin-like and metalloprotease (reprolysin type) with thrombospondin type 1 motif, 5 (agg |
| 879 | Serine/threonine kinase 17b (apoptosis-inducing) \|\| BC052561 \|\| 2q32.3 |
| 880 | SH3-domain binding protein 4 \|\| BC057396 \|\| 2q37.1-q37.2 |
| 881 | Family with sequence similarity 49, member B \|\| CR749628 \|\| 8q24.21 |
| 882 | Abl-interactor 1 \|\| NM_005470 \|\| 10p11.2 |
| 883 | Mitochondrial ribosomal protein S17 \|\| AK026553 \|\| 7p11 |
| 884 | 3′-phosphoadenosine 5′-phosphosulfate synthase 1 \|\| NM_005443 \|\| 4q24 |
| 885 | TBC1 domain family, member 8 (with GRAM domain) \|\| AB024057 \|\| 2q11.2 |
| 886 | Solute carrier family 2 (facilitated glucose transporter), member 1 \|\| NM_006516 \|\| 1p35-p31.3 |
| 887 | Signal recognition particle 9 kDa \|\| BC064351 \|\| 1q42.12 |
| 888 | Intercellular adhesion molecule 1 (CD54), human rhinovirus receptor \|\| BC015969 \|\| 19p13.3-p1 [symbol] |
| 889 | Olfactomedin-like 3 \|\| AK075544 \|\| 1p13.2 |
| 890 | RNA binding motif protein 7 \|\| AB209753 \|\| 11q23.1-q23.2 |
| 891 | F-box protein 16 \|\| NM_172366 \|\| 8p21.1 |
| 892 | Scavenger receptor class B, member 1 \|\| AB209436 \|\| 12q24.31 |
| 893 | DNA segment on chromosome 21 (unique) 2056 expressed sequence \|\| NM_003683 \|\| 21q22.3 |
| 894 | Armadillo repeat containing, X-linked 2 \|\| BC052628 \|\| Xq21.33-q22.2 |
| 895 | SCY1-like 3 (S. cerevisiae) \|\| BX647352 \|\| 1q24.2 |
| 896 | Protocadherin 8 \|\| AF061573 \|\| 13q14.3-q21.1 |
| 897 | Cyclin D1 (PRAD1: parathyroid adenomatosis 1) \|\| NM_053056 \|\| 11q13 |
| 898 | Hypothetical protein MGC52110 \|\| AK128366 \|\| |
| 899 | PEST-containing nuclear protein \|\| BX647886 \|\| 3q12.3 |
| 900 | Myelin protein zero-like 1 \|\| NM_003953 \|\| 1q24.2 |
| 901 | Chromosome 18 open reading frame 43 \|\| CR627465 \|\| 18p11.21 |
| 902 | Dual specificity phosphatase 1 \|\| AK127679 \|\| 5q34 |
| 903 | CD53 antigen \|\| BC035456 \|\| 1p13 |
| 904 | Sirtuin (silent mating type information regulation 2 homolog) 1 (S. cerevisiae) \|\| NM_012238 \|\| 10 |
| 905 | Glutaredoxin 2 \|\| BM908128 \|\| 1q31.2-q31.3 |
| 906 | Erbb2 interacting protein \|\| NM_18695 \|\| 5q12.3 |
| 907 | Cystatin B (stefin B) \|\| CR591371 \|\| 21q22.3 |
| 908 | Polymerase (DNA directed), theta \|\| CR936627 \|\| 3q13.33 |
| 909 | Mitogen-activated protein kinase kinase 4 \|\| AK131544 \|\| 17p11.2 |
| 910 | Collagen, type IV, alpha 5 (Alport syndrome) \|\| NM_033380 \|\| Xq22 |
| 911 | Nuclear receptor coactivator 7 \|\| AL834442 \|\| 6q22.32 |
| 912 | Hypothetical protein FLJ20152 \|\| AL832438 \|\| 5p15.1 |
| 913 | Hypothetical protein MGC45780 \|\| NM_173833 \|\| 8p21.1 |
| 914 | Ribonuclease H2, large subunit \|\| CR619517 \|\| 19p13.13 |
| 915 | CGI-12 protein \|\| NM_015942 \|\| 8q22.1 |
| 916 | Decorin \|\| NM_001920 \|\| 12q13.2 |
| 917 | Hypothetical protein MGC17299 \|\| BC072393 \|\| 1p34.2 |
| 918 | Phosphatidylinositol glycan, class T \|\| AK123590 \|\| 20q12-q13.12 |
| 919 | CD68 antigen \|\| BC015557 \|\| 17p13 |
| 920 | S100 calcium binding protein P \|\| CA313584 \|\| 4p16 |
| 921 | High mobility group AT-hook 1 \|\| BC078664 \|\| 6p21 |
| 922 | Insulin-like growth factor binding protein 2, 36 kDa \|\| AB209509 \|\| 2q33-q34 |
| 923 | Neuromedin U \|\| BF034907 \|\| 4q12 |
| 924 | Poly(A) binding protein interacting protein 2 \|\| BC048106 \|\| 5q31.2 |
| 925 | GTP cyclohydrolase I feedback regulator \|\| BC027487 \|\| 15q15 |
| 926 | Solute carrier family 35, member B1 \|\| AK124975 \|\| 17q21.33 |
| 927 | UDP-galactose-4-epimerase \|\| AK057302 \|\| 1p36-p35 |
| 928 | Component of oligomeric golgi complex 2 \|\| AL832190 \|\| 1q42.2 |
| 929 | Signal transducer and activator of transcription 1, 91 kDa \|\| NM_007315 \|\| 2q32.2 |
| 930 | Sine oculis homeobox homolog 1 (Drosophila) \|\| AK093780 \|\| 14q23.1 |
| 931 | Choline phosphotransferase 1 \|\| AK025141 \|\| 12q |
| 932 | Chromosome 4 open reading frame 16 \|\| BX647702 \|\| 4q25 |
| 933 | CCAAT/enhancer binding protein (C/EBP), gamma \|\| NM_001806 \|\| 19q13.11 |
| 934 | G protein-coupled receptor 56 \|\| NM_201524 \|\| 16q13 |
| 935 | CUE domain containing 1 \|\| CR627470 \|\| 17q23.2 |
| 936 | Low density lipoprotein receptor-related protein 10 \|\| NM_014045 \|\| 14q11.2 |
| 937 | CD163 antigen \|\| Z22970 \|\| 12p13.3 |
| 938 | Eukaryotic translation initiation factor 4E binding protein 1 \|\| BM564526 \|\| 8p12 |
| 939 | Leupaxin \|\| BC034230 \|\| 11q12.1 |
| 940 | Myxovirus (influenza virus) resistance 1, interferon-inducible protein p78 (mouse) \|\| AK096355 \|\| |
| 941 | Low molecular mass ubiquinone-binding protein (9.5 kD) \|\| BM701597 \|\| 5q31.1 |
| 942 | Chromosome 20 open reading frame 17 \|\| NM_173485 \|\| 20q13.2 |
| 943 | Selenophosphate synthetase 1 \|\| AK125066 \|\| 10p14 |
| 944 | Manic fringe homolog (Drosophila) \|\| U94352 \|\| 22q12 |
| 945 | HMBA-inducible \|\| AB021179 \|\| 17q21.31 |
| 946 | Dynein, cytoplasmic, heavy polypeptide 2 \|\| XM_370652 \|\| 11q21-q22.1 |
| 947 | START domain containing 3 \|\| AL831952 \|\| 17q11-q12 |
| 948 | Calsyntenin 2 \|\| AJ278018 \|\| 3q23-q24 |
| 949 | Programmed cell death 5 \|\| AB209040 \|\| 19q12-q13.1 |
| 950 | Influenza virus NS1A binding protein \|\| NM_016389 \|\| 1q25.1-q31.1 |
| 951 | Annexin A3 \|\| AB209868 \|\| 4q13-q22 |
| 952 | C-type lectin domain family 11, member A \|\| BM719769 \|\| 19q13.3 |
| 953 | Hypothetical protein LOC130576 \|\| NM_177964 \|\| 2q23.3 |
| 954 | Protein disulfide isomerase-associated 6 \|\| AK127433 \|\| 2p25.1 |
| 955 | Surfeit 1 \|\| BM923055 \|\| 9q34.2 |
| 956 | Septin 10 \|\| AB208875 \|\| 2q13 |
| 957 | Discoidin domain receptor family, member 1 \|\| NM_013994 \|\| 6p21.3 |
| 958 | RA-regulated nuclear matrix-associated protein \|\| NM_016448 \|\| |
| 959 | Lipase A, lysosomal acid, cholesterol esterase (Wolman disease) \|\| AK091558 \|\| 10q23.2-q23.3 |
| 960 | Cytochrome b5 reductase b5R.2 \|\| AB209000 \|\| 11p15.4 |
| 961 | COMM domain containing 10 \|\| BC036897 \|\| 5q23.1 |
| 962 | SRY (sex determining region Y)-box B \|\| NM_014587 \|\| 16p13.3 |
| 963 | FXYD domain containing ion transport regulator 3 \|\| BF676327 \|\| 19q13.11-q13.12 |
| 964 | Adenosine deaminase \|\| BM809993 \|\| 20q12-q13.11 |
| 965 | Serine/threonine kinase 38 like \|\| AB023182 \|\| 12p11.23 |
| 966 | Schwannomin interacting protein 1 \|\| BX648179 \|\| 3q25.32-q25.33 |
| 967 | Factor for adipocyte differentiation 15B \|\| BC036122 \|\| 1p22.2 |
| 968 | Solute carrier family 1 (glial high affinity glutamate transporter), member 2 \|\| AY066021 \|\| 11p13- |
| 969 | Tryptophanyl-tRNA synthetase \|\| NM_004184 \|\| 14q32.31 |
| 970 | Chromosome 13 open reading frame 21 \|\| AK123212 \|\| 13q14.11 |
| 971 | Chromosome 6 open reading frame 85 \|\| NM_021945 \|\| 6p25.2 |
| 972 | Hypothetical gene CG01B \|\| AL832677 \|\| 13q12-q13 |
| 973 | Diacylglycerol kinase, delta 130 kDa \|\| NM_152879 \|\| 2q37.1 |
| 974 | MOB1, Mps One Binder kinase activator-like 2B (yeast) \|\| NM_024761 \|\| 9p21.2 |
| 975 | Zinc finger homeobox 1b \|\| NM_014795 \|\| 2q22 |
| 976 | Chromosome 5 open reading frame 3 \|\| CR749447 \|\| 5q31-q33 |
| 977 | CSE1 chromosome segregation 1-like (yeast) \|\| NM_001316 \|\| 20q13 |
| 978 | Retinoblastoma binding protein 8 \|\| NM_002894 \|\| 18q11.2 |
| 979 | Alpha-methylacyl-CoA racemase \|\| CR616479 \|\| 5p13.2-q11.1 |
| 980 | Histone 1, H2ab \|\| CK130254 \|\| 6p21.3 |
| 981 | B-factor, properdin \|\| NM_001710 \|\| 6p21.3 |
| 982 | Peroxiredoxin 4 \|\| BM674623 \|\| Xp22.11 |
| 983 | Malic enzyme 2, NAD(+)-dependent, mitochondrial \|\| NM_002396 \|\| 6p25-p24 |
| 984 | Proteasome (prosome, macropain) subunit, beta type, 4 \|\| CR601408 \|\| 1q21 |
| 985 | AHNAK nucleoprotein (desmoyokin) \|\| NM_001620 \|\| 11q12.2 |
| 986 | ATPase family, AAA domain containing 3A \|\| AK092633 \|\| 1p36.33 |
| 987 | Protein phosphatase 2, regulatory subunit B (B56), alpha isoform \|\| NM_006243 \|\| 1q32.2-q32.3 |
| 988 | Cholesterol 25-hydroxylase \|\| NM_003956 \|\| 10q23 |
| 989 | Hypoxanthine phosphoribosyltransferase 1 (Lesch-Nyhan syndrome) \|\| NM_000194 \|\| Xq26.1 |
| 990 | BCL2-associated athanogene 2 \|\| AK023735 \|\| 6p12.3-p11.2 |
| 991 | Solute carrier family 7 (cationic amino acid transporter, y+ system), member 8 \|\| Y18483 \|\| 14q11 |
| 992 | Hypothetical protein FLJ22833 \|\| AL832659 \|\| 2q32.3 |
| 993 | Hypothetical protein FLJ90586 \|\| BC035517 \|\| 7q34 |
| 994 | Procollagen-lysine 1, 2-oxoglutarate 5-dioxygenase 1 \|\| NM_000302 \|\| 1p36.3-p36.2 |
| 995 | Adenosylmethionine decarboxylase 1 \|\| BC041345 \|\| 6q21-q22 |
| 996 | Protein S (alpha) \|\| M14338 \|\| 3q11.2 |
| 997 | Rho GTPase activating protein 4 \|\| BC052303 \|\| Xq28 |
| 998 | Proteasome (prosome, macropain) subunit, beta type, 2 \|\| BM545813 \|\| 1p34.2 |
| 999 | Soc-2 suppressor of clear homolog (C. elegans) \|\| BC044752 \|\| 10q25 |
| 1000 | Aldehyde dehydrogenase 4 family, member A1 \|\| NM_003748 \|\| 1p36 |
| 1001 | HSPC171 protein \|\| BF204699 \|\| 16q22.1 |
| 1002 | Two pore segment channel 1 \|\| AB032995 \|\| 12q24.13 |
| 1003 | Necdin homolog (mouse) \|\| NM_002487 \|\| 15q11.2-q12 |
| 1004 | Potassium channel modulatory factor 1 \|\| NM_020122 \|\| 2p11.2 |
| 1005 | Likely ortholog of mouse hypoxia induced gene 1 \|\| AL833541 \|\| 3p22.1 |
| 1006 | Solute carrier family 30 (zinc transporter), member 5 \|\| BX537394 \|\| 5q12.1 |
| 1007 | Cathepsin S \|\| NM_004079 \|\| 1q21 |
| 1008 | Choline dehydrogenase \|\| AK055402 \|\| 3p21.1 |
| 1009 | Breast carcinoma amplified sequence 4 \|\| BC056883 \|\| 20q13.13 |
| 1010 | DKFZp564J157 protein \|\| BE906094 \|\| 12q12 |
| 1011 | Retinoblastoma 1 (including osteosarcoma) \|\| L41870 \|\| 13q14.2 |
| 1012 | Actin-like 6A \|\| NM_178042 \|\| 3q26.33 |
| 1013 | Kinesin family member 3C \|\| BX571741 \|\| 2p23 |
| 1014 | Related RAS viral (r-ras) oncogene homolog 2 \|\| BQ228116 \|\| 11p15.2 |
| 1015 | Absent in melanoma 2 \|\| BC010940 \|\| 1q22 |
| 1016 | Chromosome 5 open reading frame 5 \|\| AF251038 \|\| 5q31 |
| 1017 | Pyrimidinergic receptor P2Y, G-protein coupled, 6 \|\| NM_004154 \|\| 11q13.5 |
| 1018 | Hypothetical protein FLJ10579 \|\| AK123282 \|\| 15q15.1 |
| 1019 | Enah/Vasp-like \|\| AL133642 \|\| 14q32.2 |
| 1020 | Sarcoma antigen NY-SAR-48 \|\| AK130803 \|\| 19p13.11 |
| 1021 | Lactamase, beta 2 \|\| NM_016027 \|\| 8p22-q22.3 |
| 1022 | Echinoderm microtubule associated protein like 4 \|\| NM_019063 \|\| 2p22-p21 |
| 1023 | Peroxisomal biogenesis factor 19 \|\| NM_002857 \|\| 1q22 |
| 1024 | Nucleobindin 2 \|\| AK128739 \|\| 11p15.1-p14 |
| 1025 | Putative membrane protein \|\| BF680501 \|\| 1q22-q25 |
| 1026 | Phospholipase A2, group IVA (cytosolic, calcium-dependent) \|\| M68874 \|\| 1q25 |
| 1027 | Cathepsin L \|\| AK055599 \|\| 9q21-q22 |
| 1028 | Ras-related GTP binding D \|\| AL137502 \|\| 6q15-q16 |
| 1029 | Protein kinase, cAMP-dependent, regulatory, type I, alpha (tissue specific extinguisher 1) \|\| CR7 [symbol] |
| 1030 | CD28 antigen (Tp44) \|\| NM_006139 \|\| 2q33 |
| 1031 | KIAA0133 \|\| NM_014777 \|\| 1q42.13 |
| 1032 | Serine dehydratase-like \|\| BC009849 \|\| 12q24.13 |
| 1033 | Matrix metalloproteinase 1 (interstitial collagenase) \|\| BC013875 \|\| 11q22.3 |
| 1034 | Solute carrier family 25 (mitochondrial carrier, Aralar), member 12 \|\| AJ496568 \|\| 2q24 |
| 1035 | Down syndrome critical region gene 1 \|\| AY325903 \|\| 21q22.1-q22.2 |
| 1036 | Heme oxygenase (decycling) 1 \|\| BG165629 \|\| 22q12 |
| 1037 | Lipase, hormone-sensitive \|\| BC070041 \|\| 19q13.2 |
| 1038 | ARP3 actin-related protein 3 homolog (yeast) \|\| BC044590 \|\| 2q14.1 |
| 1039 | Dickkopf homolog 3 (Xenopus laevis) \|\| NM_015881 \|\| 11p15.2 |
| 1040 | Laminin, beta 2 (laminin S) \|\| NM_002292 \|\| 3p21 |
| 1041 | Nudix (nucleoside diphosphate linked moiety X)-type motif 4 \|\| NM_199040 \|\| |
| 1042 | Sarcoma amplified sequence \|\| BX647402 \|\| 12q13.3 |
| 1043 | Enoyl Coenzyme A hydratase domain containing 2 \|\| BX647186 \|\| 1p32.3 |
| 1044 | Optineurin \|\| NM_001008211 \|\| 10p13 |
| 1045 | Secreted frizzled-related protein 4 \|\| AF026692 \|\| 7p14.1 |
| 1046 | Glutathione transferase zeta 1 (maleylacetoacetate isomerase) \|\| AB209360 \|\| 14q24.3 |
| 1047 | Sterol carrier protein 2 \|\| AB208789 \|\| 1p32 |
| 1048 | KIAA0649 \|\| NM_014811 \|\| 9q34.3 |
| 1049 | Serine palmitoyltransferase, long chain base subunit 2 \|\| NM_004863 \|\| 14q24.3-q31 |
| 1050 | Stannin \|\| NM_003498 \|\| 16p13 |
| 1051 | Chondroadherin \|\| NM_001267 \|\| 17q21.33 |
| 1052 | GCIP-interacting protein p29 \|\| BC015824 \|\| 1p36.11 |
| 1053 | Transcription elongation factor A (SII)-like 4 \|\| CR594284 \|\| Xq22.2 |
| 1054 | Glycoprotein M6B \|\| NM_001001995 \|\| Xp22.2 |
| 1055 | HIV-1 Tat interactive protein 2, 30 kDa \|\| NM_006410 \|\| 11p15.1 |
| 1056 | Mitogen-activated protein kinase 6 \|\| NM_002748 \|\| 15q21 |
| 1057 | Regulator of G-protein signalling 2, 24 kDa \|\| BC042755 \|\| 1q31 |
| 1058 | Family with sequence similarity 49, member A \|\| AK055334 \|\| 2p24.3-p24.2 |
| 1059 | Sorting nexin 1 \|\| AB209013 \|\| 15q22.31 |
| 1060 | Kruppel-like factor 2 (lung) \|\| BM549806 \|\| 19p13.13-p13.11 |
| 1061 | Heat shock 70 kDa protein 14 \|\| BC026226 \|\| 10p13 |
| 1062 | Kruppel-like factor 5 (intestinal) \|\| AF132818 \|\| 13q22.1 |
| 1063 | Cathepsin C \|\| BX537913 \|\| 11q14.1-q14.3 |
| 1064 | Shugoshin-like 1 (S. pombe) \|\| AB187578 \|\| 3p24.3 |
| 1065 | Ankyrin repeat domain 34 \|\| AK094282 \|\| 1q21.1 |
| 1066 | FP15737 \|\| AF495725 \|\| |
| 1067 | SH2 domain binding protein 1 (tetratricopepttde repeat containing) \|\| BC058914 \|\| 11p15.3 |
| 1068 | Biliverdin reductase B (flavin reductase (NADPH)) \|\| BF341546 \|\| 19q13.1-q13.2 |
| 1069 | Pre-B-cell leukemia transcription factor 1 \|\| CR749446 \|\| 1q23 |
| 1070 | UDP-GlcNAc:betaGal beta-1,3-N-acetylglucosaminyltransferase 6 \|\| NM_006876 \|\| 11q13.2 |
| 1071 | Laminin, beta 3 \|\| NM_001017402 \|\| 1q32 |
| 1072 | Clathrin, heavy polypeptide (Hc) \|\| NM_004859 \|\| 17q11-qter |
| 1073 | Ornithine aminotransferase (gyrate atrophy) \|\| AB208817 \|\| 10q26 |
| 1074 | Pleckstrin homology-like domain, family B, member 2 \|\| AL832205 \|\| 3q13.2 |
| 1075 | Chromosome 14 open reading frame 58 \|\| AY260577 \|\| 14q24.3 |
| 1076 | Serine hydroxymethyltransferase 2 (mitochondrial) \|\| AK055053 \|\| 12q12-q14 |
| 1077 | Sparc/osteonectin, cwcv and kazal-like domains proteoglycan (testican) 2 \|\| NM_014767 \|\| 10pte |
| 1078 | MBC3205 \|\| AK127147 \|\| 19p13.2 |
| 1079 | F-box protein 31 \|\| AF318348 \|\| 16q24.2 |
| 1080 | MGC16028 similar to RIKEN cDNA 1700019E19 gene \|\| BU733407 \|\| 14q24.3 |
| 1081 | PTK9 protein tyrosine kinase 9 \|\| NM_198974 \|\| 12q12 |
| 1082 | Dystrophin (muscular dystrophy, Duchenne and Becker types) \|\| NM_004010 \|\| Xp21.2 |
| 1083 | Cathepsin G \|\| BU621869 \|\| 14q11.2 |
| 1084 | Elastin microfibril interfacer 2 \|\| AF270513 \|\| 18p11.3 |
| 1085 | WW domain containing transcription regulator 1 \|\| AL833852 \|\| 3q23-q24 |
| 1086 | Hypothetical protein FLJ14525 \|\| BC066649 \|\| 1q42.13-q43 |
| 1087 | RecQ protein-like 4 \|\| BC020496 \|\| 8q24.3 |
| 1088 | Thrombomodulin \|\| NM_000361 \|\| 20p12-cen |
| 1089 | RAP2A, member of RAS oncogene family \|\| NM_021033 \|\| 13q34 |
| 1090 | Reproduction 8 \|\| NM_005671 \|\| 8p12-p11.2 |
| 1091 | Nuclear autoantigenic sperm protein (histone-binding) \|\| AY700118 \|\| 1p34.1 |
| 1092 | Hypothetical protein FLJ11273 \|\| NM_018374 \|\| 7p21.3 |
| 1093 | Hypothetical protein MGC10986 \|\| NM_030576 \|\| 17q23.3 |
| 1094 | Ganglioside-induced differentiation-associated protein 1 \|\| AL110252 \|\| 8q21.11 |
| 1095 | GDP dissociation inhibitor 1 \|\| AK123405 \|\| Xq28 |
| 1096 | Translocase of outer mitochondrial membrane 40 homolog (yeast) \|\| BC047528 \|\| 19q13 |
| 1097 | LAG1 longevity assurance homolog 2 (S. cerevisiae) \|\| NM_181746 \|\| 1q21.2 |
| 1098 | SH3 domain protein D19 \|\| BX647422 \|\| 4q31.3 |
| 1099 | ATP synthase, H+ transporting, mitochondrial F0 complex, subunit c (subunit 9), isoform 2 \|\| CR! |
| 1100 | Solute carrier family 16 (monocarboxylic acid transporters), member 5 \|\| AK092512 \|\| 17q25.1 |
| 1101 | Nudix (nucleoside diphosphate linked moiety X)-type motif 16-like 1 \|\| BQ679635 \|\| 16p13.3 |
| 1102 | Cytochrome P450, family 26, subfamily A, polypeptide 1 \|\| AK027560 \|\| 10q23-q24 |

| 7P-SAM-Order | ProbeID | ProbeID |
|---|---|---|
| 1 | AGI_HUM1_OLIGO_A_23_P37127 | |
| 2 | AGI_HUM1_OLIGO_A_23_P89249 | |
| 3 | AGI_HUM1_OLIGO_A_23_P113111 | |
| 4 | AGI_HUM1_OLIGO_A_23_P148249 | |
| 5 | AGI_HUM1_OLIGO_A_23_P120845 | |
| 6 | AGI_HUM1_OLIGO_A_23_P390504 | |
| 7 | AGI_HUM1_OLIGO_A_23_P257296 | |
| 8 | AGI_HUM1_OLIGO_A_23_P95596 | |
| 9 | AGI_HUM1_OLIGO_A_23_P42811 | |
| 10 | AGI_HUM1_OLIGO_A_23_P75056 | |
| 11 | AGI_HUM1_OLIGO_A_23_P31407 | |
| 12 | AGI_HUM1_OLIGO_A_23_P309739 | AGI_HUM1_OLIGO_A_23_P59308 |
| 13 | AGI_HUM1_OLIGO_A_23_P105144 | |
| 14 | AGI_HUM1_OLIGO_A_23_P124417 | |
| 15 | AGI_HUM1_OLIGO_A_23_P50167 | |
| 16 | AGI_HUM1_OLIGO_A_23_P61346 | |
| 17 | AGI_HUM1_OLIGO_A_23_P100001 | |
| 18 | AGI_HUM1_OLIGO_A_23_P50108 | |
| 19 | AGI_HUM1_OLIGO_A_23_P15101 | |
| 20 | AGI_HUM1_OLIGO_A_23_P151956 | AGI_HUM1_OLIGO_A_23_P163336 |
| 21 | AGI_HUM1_OLIGO_A_23_P79429 | |
| 22 | AGI_HUM1_OLIGO_A_23_P118815 | |
| 23 | AGI_HUM1_OLIGO_A_23_P157099 | |
| 24 | AGI_HUM1_OLIGO_A_23_P115872 | |
| 25 | AGI_HUM1_OLIGO_A_23_P90510 | |
| 26 | AGI_HUM1_OLIGO_A_23_P65757 | |
| 27 | AGI_HUM1_OLIGO_A_23_P156498 | |
| 28 | AGI_HUM1_OLIGO_A_23_P1682 | |
| 29 | AGI_HUM1_OLIGO_A_23_P259586 | |
| 30 | AGI_HUM1_OLIGO_A_23_P146456 | |
| 31 | AGI_HUM1_OLIGO_A_23_P108910 | |
| 32 | AGI_HUM1_OLIGO_A_23_P144384 | |
| 33 | AGI_HUM1_OLIGO_A_23_P127220 | |
| 34 | AGI_HUM1_OLIGO_A_23_P154400 | |
| 35 | AGI_HUM1_OLIGO_A_23_P91081 | |
| 36 | AGI_HUM1_OLIGO_A_23_P149195 | |
| 37 | AGI_HUM1_OLIGO_A_23_P8702 | |
| 38 | AGI_HUM1_OLIGO_A_23_P251421 | |
| 39 | AGI_HUM1_OLIGO_A_23_P4714 | |
| 40 | AGI_HUM1_OLIGO_A_23_P112634 | |
| 41 | AGI_HUM1_OLIGO_A_23_P201551 | |
| 42 | AGI_HUM1_OLIGO_A_23_P163989 | |
| 43 | AGI_HUM1_OLIGO_A_23_P143207 | |
| 44 | AGI_HUM1_OLIGO_A_23_P113317 | |
| 45 | AGI_HUM1_OLIGO_A_23_P80918 | |
| 46 | AGI_HUM1_OLIGO_A_23_P9442 | |
| 47 | AGI_HUM1_OLIGO_A_23_P375 | |
| 48 | AGI_HUM1_OLIGO_A_23_P160377 | |
| 49 | AGI_HUM1_OLIGO_A_23_P99853 | |
| 50 | AGI_HUM1_OLIGO_A_23_P74115 | |
| 51 | AGI_HUM1_OLIGO_A_23_P122650 | AGI_HUM1_OLIGO_A_23_P99324 |
| 52 | AGI_HUM1_OLIGO_A_23_P140434 | |
| 53 | AGI_HUM1_OLIGO_A_23_P257111 | |
| 54 | AGI_HUM1_OLIGO_A_23_P57379 | |
| 55 | AGI_HUM1_OLIGO_A_23_P213680 | |
| 56 | AGI_HUM1_OLIGO_A_23_P206441 | |
| 57 | AGI_HUM1_OLIGO_A_23_P31073 | |
| 58 | AGI_HUM1_OLIGO_A_23_P7791 | |
| 59 | AGI_HUM1_OLIGO_A_23_P34788 | |
| 60 | AGI_HUM1_OLIGO_A_23_P136222 | |
| 61 | AGI_HUM1_OLIGO_A_23_P131866 | |
| 62 | AGI_HUM1_OLIGO_A_23_P123193 | |
| 63 | AGI_HUM1_OLIGO_A_23_P142872 | |
| 64 | AGI_HUM1_OLIGO_A_23_P132378 | |
| 65 | AGI_HUM1_OLIGO_A_23_P143184 | |
| 66 | AGI_HUM1_OLIGO_A_23_P204630 | |
| 67 | AGI_HUM1_OLIGO_A_23_P152791 | |
| 68 | AGI_HUM1_OLIGO_A_23_P44964 | |
| 69 | AGI_HUM1_OLIGO_A_23_P167005 | |
| 70 | AGI_HUM1_OLIGO_A_23_P91669 | |
| 71 | AGI_HUM1_OLIGO_A_23_P218584 | |
| 72 | AGI_HUM1_OLIGO_A_23_P80032 | |
| 73 | AGI_HUM1_OLIGO_A_23_P65110 | |
| 74 | AGI_HUM1_OLIGO_A_23_P167607 | |
| 75 | AGI_HUM1_OLIGO_A_23_P115482 | |
| 76 | AGI_HUM1_OLIGO_A_23_P101972 | |
| 77 | AGI_HUM1_OLIGO_A_23_P14515 | |
| 78 | AGI_HUM1_OLIGO_A_23_P206059 | |
| 79 | AGI_HUM1_OLIGO_A_23_P111194 | |
| 80 | AGI_HUM1_OLIGO_A_23_P115392 | |
| 81 | AGI_HUM1_OLIGO_A_23_P10121 | |
| 82 | AGI_HUM1_OLIGO_A_23_P168610 | |
| 83 | AGI_HUM1_OLIGO_A_23_P92441 | |
| 84 | AGI_HUM1_OLIGO_A_23_P35219 | |
| 85 | AGI_HUM1_OLIGO_A_23_P146565 | |
| 86 | AGI_HUM1_OLIGO_A_23_P63402 | |
| 87 | AGI_HUM1_OLIGO_A_23_P88331 | |
| 88 | AGI_HUM1_OLIGO_A_23_P102391 | |
| 89 | AGI_HUM1_OLIGO_A_23_P100344 | |
| 90 | AGI_HUM1_OLIGO_A_23_P23303 | |
| 91 | AGI_HUM1_OLIGO_A_23_P256956 | |
| 92 | AGI_HUM1_OLIGO_A_23_P130027 | |
| 93 | AGI_HUM1_OLIGO_A_23_P60024 | AGI_HUM1_OLIGO_A_23_P7636 |
| 94 | AGI_HUM1_OLIGO_A_23_P204296 | |
| 95 | AGI_HUM1_OLIGO_A_23_P203115 | |
| 96 | AGI_HUM1_OLIGO_A_23_P85780 | |
| 97 | AGI_HUM1_OLIGO_A_23_P152593 | |
| 98 | AGI_HUM1_OLIGO_A_23_P49878 | |
| 99 | AGI_HUM1_OLIGO_A_23_P256473 | |
| 100 | AGI_HUM1_OLIGO_A_23_P74349 | |
| 101 | AGI_HUM1_OLIGO_A_23_P17420 | |
| 102 | AGI_HUM1_OLIGO_A_23_P38167 | |
| 103 | AGI_HUM1_OLIGO_A_23_P132910 | |
| 104 | AGI_HUM1_OLIGO_A_23_P142322 | |
| 105 | AGI_HUM1_OLIGO_A_23_P52017 | |
| 106 | AGI_HUM1_OLIGO_A_23_P51085 | |
| 107 | AGI_HUM1_OLIGO_A_23_P211007 | |
| 108 | AGI_HUM1_OLIGO_A_23_P200015 | |
| 109 | AGI_HUM1_OLIGO_A_23_P133954 | |
| 110 | AGI_HUM1_OLIGO_A_23_P109539 | |
| 111 | AGI_HUM1_OLIGO_A_23_P56709 | |
| 112 | AGI_HUM1_OLIGO_A_23_P110122 | |
| 113 | AGI_HUM1_OLIGO_A_23_P200222 | |
| 114 | AGI_HUM1_OLIGO_A_23_P100642 | |
| 115 | AGI_HUM1_OLIGO_A_23_P59613 | |
| 116 | AGI_HUM1_OLIGO_A_23_P68610 | |
| 117 | AGI_HUM1_OLIGO_A_23_P118203 | |
| 118 | AGI_HUM1_OLIGO_A_23_P110901 | |
| 119 | AGI_HUM1_OLIGO_A_23_P258462 | |
| 120 | AGI_HUM1_OLIGO_A_23_P109636 | |
| 121 | AGI_HUM1_OLIGO_A_23_P38824 | |
| 122 | AGI_HUM1_OLIGO_A_23_P168882 | |
| 123 | AGI_HUM1_OLIGO_A_23_P132088 | |
| 124 | AGI_HUM1_OLIGO_A_23_P141114 | AGI_HUM1_OLIGO_A_23_P152505 |
| 125 | AGI_HUM1_OLIGO_A_23_P8900 | |
| 126 | AGI_HUM1_OLIGO_A_23_P160406 | |
| 127 | AGI_HUM1_OLIGO_A_23_P67529 | |
| 128 | AGI_HUM1_OLIGO_A_23_P133049 | |
| 129 | AGI_HUM1_OLIGO_A_23_P22378 | |
| 130 | AGI_HUM1_OLIGO_A_23_P205531 | |
| 131 | AGI_HUM1_OLIGO_A_23_P108948 | |
| 132 | AGI_HUM1_OLIGO_A_23_P21706 | AGI_HUM1_OLIGO_A_23_P33103 |
| 133 | AGI_HUM1_OLIGO_A_23_P259641 | |
| 134 | AGI_HUM1_OLIGO_A_23_P140450 | |
| 135 | AGI_HUM1_OLIGO_A_23_P42279 | |
| 136 | AGI_HUM1_OLIGO_A_23_P136355 | |
| 137 | AGI_HUM1_OLIGO_A_23_P4494 | |
| 138 | AGI_HUM1_OLIGO_A_23_P669699 | |
| 139 | AGI_HUM1_OLIGO_A_23_P155477 | |
| 140 | AGI_HUM1_OLIGO_A_23_P151150 | |
| 141 | AGI_HUM1_OLIGO_A_23_P89509 | |
| 142 | AGI_HUM1_OLIGO_A_23_P204100 | |
| 143 | AGI_HUM1_OLIGO_A_23_P125265 | |
| 144 | AGI_HUM1_OLIGO_A_23_P166922 | |
| 145 | AGI_HUM1_OLIGO_A_23_P88731 | |
| 146 | AGI_HUM1_OLIGO_A_23_P88740 | |
| 147 | AGI_HUM1_OLIGO_A_23_P134176 | |
| 148 | AGI_HUM1_OLIGO_A_23_P33898 | |
| 149 | AGI_HUM1_OLIGO_A_23_P107051 | |
| 150 | AGI_HUM1_OLIGO_A_23_P122197 | |
| 151 | AGI_HUM1_OLIGO_A_23_P25069 | |
| 152 | AGI_HUM1_OLIGO_A_23_P217339 | |
| 153 | AGI_HUM1_OLIGO_A_23_P145104 | |
| 154 | AGI_HUM1_OLIGO_A_23_P34766 | |
| 155 | AGI_HUM1_OLIGO_A_23_P147388 | AGI_HUM1_OLIGO_A_23_P95441 |
| 156 | AGI_HUM1_OLIGO_A_23_P15516 | |
| 157 | AGI_HUM1_OLIGO_A_23_P82699 | |
| 158 | AGI_HUM1_OLIGO_A_23_P1043 | |
| 159 | AGI_HUM1_OLIGO_A_23_P24716 | |
| 160 | AGI_HUM1_OLIGO_A_23_P88404 | |
| 161 | AGI_HUM1_OLIGO_A_23_P12343 | |
| 162 | AGI_HUM1_OLIGO_A_23_P138938 | |
| 163 | AGI_HUM1_OLIGO_A_23_P106675 | |
| 164 | AGI_HUM1_OLIGO_A_23_P18559 | |
| 165 | AGI_HUM1_OLIGO_A_23_P48835 | |
| 166 | AGI_HUM1_OLIGO_A_23_P12874 | |
| 167 | AGI_HUM1_OLIGO_A_23_P200866 | |
| 168 | AGI_HUM1_OLIGO_A_23_P68759 | |
| 169 | AGI_HUM1_OLIGO_A_23_P100632 | |
| 170 | AGI_HUM1_OLIGO_A_23_P110685 | |
| 171 | AGI_HUM1_OLIGO_A_23_P200310 | |
| 172 | AGI_HUM1_OLIGO_A_23_P88630 | |
| 173 | AGI_HUM1_OLIGO_A_23_P107421 | |
| 174 | AGI_HUM1_OLIGO_A_23_P20275 | |
| 175 | AGI_HUM1_OLIGO_A_23_P54556 | |
| 176 | AGI_HUM1_OLIGO_A_23_P138507 | |
| 177 | AGI_HUM1_OLIGO_A_23_P18579 | |
| 178 | AGI_HUM1_OLIGO_A_23_P8834 | |
| 179 | AGI_HUM1_OLIGO_A_23_P107611 | |
| 180 | AGI_HUM1_OLIGO_A_23_P38574 | |
| 181 | AGI_HUM1_OLIGO_A_23_P50815 | |
| 182 | AGI_HUM1_OLIGO_A_23_P163697 | |
| 183 | AGI_HUM1_OLIGO_A_23_P151267 | |
| 184 | AGI_HUM1_OLIGO_A_23_P37704 | |
| 185 | AGI_HUM1_OLIGO_A_23_P26785 | |
| 186 | AGI_HUM1_OLIGO_A_23_P134935 | |
| 187 | AGI_HUM1_OLIGO_A_23_P76402 | |
| 188 | AGI_HUM1_OLIGO_A_23_P10182 | |
| 189 | AGI_HUM1_OLIGO_A_23_P218928 | |
| 190 | AGI_HUM1_OLIGO_A_23_P133147 | |
| 191 | AGI_HUM1_OLIGO_A_23_P73801 | |
| 192 | AGI_HUM1_OLIGO_A_23_P209619 | |
| 193 | AGI_HUM1_OLIGO_A_23_P216468 | |
| 194 | AGI_HUM1_OLIGO_A_23_P71997 | |
| 195 | AGI_HUM1_OLIGO_A_23_P19543 | |
| 196 | AGI_HUM1_OLIGO_A_23_P8452 | |
| 197 | AGI_HUM1_OLIGO_A_23_P132515 | |
| 198 | AGI_HUM1_OLIGO_A_23_P92042 | |
| 199 | AGI_HUM1_OLIGO_A_23_P205134 | |
| 200 | AGI_HUM1_OLIGO_A_23_P145694 | |
| 201 | AGI_HUM1_OLIGO_A_23_P259071 | |
| 202 | AGI_HUM1_OLIGO_A_23_P254165 | |
| 203 | AGI_HUM1_OLIGO_A_23_P129786 | |
| 204 | AGI_HUM1_OLIGO_A_23_P42718 | |
| 205 | AGI_HUM1_OLIGO_A_23_P90612 | |
| 206 | AGI_HUM1_OLIGO_A_23_P156880 | |
| 207 | AGI_HUM1_OLIGO_A_23_P94840 | |
| 208 | AGI_HUM1_OLIGO_A_23_P51966 | |
| 209 | AGI_HUM1_OLIGO_A_23_P90649 | |
| 210 | AGI_HUM1_OLIGO_A_23_P4551 | |
| 211 | AGI_HUM1_OLIGO_A_23_P254472 | |
| 212 | AGI_HUM1_OLIGO_A_23_P140705 | |
| 213 | AGI_HUM1_OLIGO_A_23_P12082 | |
| 214 | AGI_HUM1_OLIGO_A_23_P254733 | |
| 215 | AGI_HUM1_OLIGO_A_23_P75589 | |
| 216 | AGI_HUM1_OLIGO_A_23_P52278 | |
| 217 | AGI_HUM1_OLIGO_A_23_P60130 | |
| 218 | AGI_HUM1_OLIGO_A_23_P105862 | |
| 219 | AGI_HUM1_OLIGO_A_23_P14432 | |
| 220 | AGI_HUM1_OLIGO_A_23_P106544 | |
| 221 | AGI_HUM1_OLIGO_A_23_P82950 | |
| 222 | AGI_HUM1_OLIGO_A_23_P160167 | |
| 223 | AGI_HUM1_OLIGO_A_23_P259692 | |
| 224 | AGI_HUM1_OLIGO_A_23_P101208 | |
| 225 | AGI_HUM1_OLIGO_A_23_P253752 | |
| 226 | AGI_HUM1_OLIGO_A_23_P50096 | |
| 227 | AGI_HUM1_OLIGO_A_23_P118894 | |
| 228 | AGI_HUM1_OLIGO_A_23_P217236 | |
| 229 | AGI_HUM1_OLIGO_A_23_P92860 | |
| 230 | AGI_HUM1_OLIGO_A_23_P156852 | |
| 231 | AGI_HUM1_OLIGO_A_23_P49155 | |
| 232 | AGI_HUM1_OLIGO_A_23_P147431 | |
| 233 | AGI_HUM1_OLIGO_A_23_P206359 | |
| 234 | AGI_HUM1_OLIGO_A_23_P96285 | |
| 235 | AGI_HUM1_OLIGO_A_23_P137856 | |
| 236 | AGI_HUM1_OLIGO_A_23_P80068 | |
| 237 | AGI_HUM1_OLIGO_A_23_P14072 | |
| 238 | AGI_HUM1_OLIGO_A_23_P29773 | |
| 239 | AGI_HUM1_OLIGO_A_23_P86100 | |
| 240 | AGI_HUM1_OLIGO_A_23_P43157 | |
| 241 | AGI_HUM1_OLIGO_A_23_P145529 | |
| 242 | AGI_HUM1_OLIGO_A_23_P206183 | |
| 243 | AGI_HUM1_OLIGO_A_23_P45475 | |
| 244 | AGI_HUM1_OLIGO_A_23_P12523 | |
| 245 | AGI_HUM1_OLIGO_A_23_P5882 | |
| 246 | AGI_HUM1_OLIGO_A_23_P70307 | |
| 247 | AGI_HUM1_OLIGO_A_23_P59375 | |
| 248 | AGI_HUM1_OLIGO_A_23_P213141 | |
| 249 | AGI_HUM1_OLIGO_A_23_P142090 | |
| 250 | AGI_HUM1_OLIGO_A_23_P216225 | |
| 251 | AGI_HUM1_OLIGO_A_23_P216068 | |
| 252 | AGI_HUM1_OLIGO_A_23_P160546 | |
| 253 | AGI_HUM1_OLIGO_A_23_P38537 | |
| 254 | AGI_HUM1_OLIGO_A_23_P202004 | |
| 255 | AGI_HUM1_OLIGO_A_23_P161507 | |
| 256 | AGI_HUM1_OLIGO_A_23_P139704 | |
| 257 | AGI_HUM1_OLIGO_A_23_P62901 | |
| 258 | AGI_HUM1_OLIGO_A_23_P158570 | |
| 259 | AGI_HUM1_OLIGO_A_23_P10031 | AGI_HUM1_OLIGO_A_23_P170037 |
| 260 | AGI_HUM1_OLIGO_A_23_P206662 | |
| 261 | AGI_HUM1_OLIGO_A_23_P25194 | |
| 262 | AGI_HUM1_OLIGO_A_23_P518 | |
| 263 | AGI_HUM1_OLIGO_A_23_P78248 | |
| 264 | AGI_HUM1_OLIGO_A_23_P146284 | |
| 265 | AGI_HUM1_OLIGO_A_23_P128967 | |
| 266 | AGI_HUM1_OLIGO_A_23_P202658 | |
| 267 | AGI_HUM1_OLIGO_A_23_P66454 | |
| 268 | AGI_HUM1_OLIGO_A_23_P19944 | |
| 269 | AGI_HUM1_OLIGO_A_23_P21495 | AGI_HUM1_OLIGO_A_23_P32896 |
| 270 | AGI_HUM1_OLIGO_A_23_P255952 | |
| 271 | AGI_HUM1_OLIGO_A_23_P73589 | |
| 272 | AGI_HUM1_OLIGO_A_23_P58321 | |
| 273 | AGI_HUM1_OLIGO_A_23_P117683 | |
| 274 | AGI_HUM1_OLIGO_A_23_P202964 | |
| 275 | AGI_HUM1_OLIGO_A_23_P51105 | |
| 276 | AGI_HUM1_OLIGO_A_23_P11800 | |
| 277 | AGI_HUM1_OLIGO_A_23_P36345 | |
| 278 | AGI_HUM1_OLIGO_A_23_P151297 | |
| 279 | AGI_HUM1_OLIGO_A_23_P50081 | |
| 280 | AGI_HUM1_OLIGO_A_23_P24444 | |
| 281 | AGI_HUM1_OLIGO_A_23_P487 | |
| 282 | AGI_HUM1_OLIGO_A_23_P82868 | |
| 283 | AGI_HUM1_OLIGO_A_23_P72411 | |
| 284 | AGI_HUM1_OLIGO_A_23_P161439 | |
| 285 | AGI_HUM1_OLIGO_A_23_P209320 | |
| 286 | AGI_HUM1_OLIGO_A_23_P63232 | |
| 287 | AGI_HUM1_OLIGO_A_23_P140069 | |
| 288 | AGI_HUM1_OLIGO_A_23_P11652 | |
| 289 | AGI_HUM1_OLIGO_A_23_P14083 | |
| 290 | AGI_HUM1_OLIGO_A_23_P209449 | |
| 291 | AGI_HUM1_OLIGO_A_23_P256413 | |
| 292 | AGI_HUM1_OLIGO_A_23_P101992 | |
| 293 | AGI_HUM1_OLIGO_A_23_P212339 | |
| 294 | AGI_HUM1_OLIGO_A_23_P167067 | |
| 295 | AGI_HUM1_OLIGO_A_23_P75430 | |
| 296 | AGI_HUM1_OLIGO_A_23_P71148 | |
| 297 | AGI_HUM1_OLIGO_A_23_P60499 | |
| 298 | AGI_HUM1_OLIGO_A_23_P59876 | |
| 299 | AGI_HUM1_OLIGO_A_23_P7245 | |
| 300 | AGI_HUM1_OLIGO_A_23_P204133 | |
| 301 | AGI_HUM1_OLIGO_A_23_P2271 | |
| 302 | AGI_HUM1_OLIGO_A_23_P18196 | |
| 303 | AGI_HUM1_OLIGO_A_23_P116123 | |
| 304 | AGI_HUM1_OLIGO_A_23_P16523 | |
| 305 | AGI_HUM1_OLIGO_A_23_P252106 | |
| 306 | AGI_HUM1_OLIGO_A_23_P134734 | |
| 307 | AGI_HUM1_OLIGO_A_23_P53193 | |
| 308 | AGI_HUM1_OLIGO_A_23_P108143 | |
| 309 | AGI_HUM1_OLIGO_A_23_P111532 | |
| 310 | AGI_HUM1_OLIGO_A_23_P137179 | |
| 311 | AGI_HUM1_OLIGO_A_23_P45917 | |
| 312 | AGI_HUM1_OLIGO_A_23_P152066 | |
| 313 | AGI_HUM1_OLIGO_A_23_P206140 | |
| 314 | AGI_HUM1_OLIGO_A_23_P78438 | |
| 315 | AGI_HUM1_OLIGO_A_23_P162719 | |
| 316 | AGI_HUM1_OLIGO_A_23_P209020 | |
| 317 | AGI_HUM1_OLIGO_A_23_P216361 | |
| 318 | AGI_HUM1_OLIGO_A_23_P215419 | |
| 319 | AGI_HUM1_OLIGO_A_23_P257704 | |
| 320 | AGI_HUM1_OLIGO_A_23_P37560 | |
| 321 | AGI_HUM1_OLIGO_A_23_P78664 | |
| 322 | AGI_HUM1_OLIGO_A_23_P258862 | |
| 323 | AGI_HUM1_OLIGO_A_23_P43490 | |
| 324 | AGI_HUM1_OLIGO_A_23_P105012 | |
| 325 | AGI_HUM1_OLIGO_A_23_P257956 | |
| 326 | AGI_HUM1_OLIGO_A_23_P208126 | |
| 327 | AGI_HUM1_OLIGO_A_23_P146654 | |
| 328 | AGI_HUM1_OLIGO_A_23_P259442 | |
| 329 | AGI_HUM1_OLIGO_A_23_P71727 | |
| 330 | AGI_HUM1_OLIGO_A_23_P73517 | |
| 331 | AGI_HUM1_OLIGO_A_23_P873 | |
| 332 | AGI_HUM1_OLIGO_A_23_P84399 | |
| 333 | AGI_HUM1_OLIGO_A_23_P137672 | |
| 334 | AGI_HUM1_OLIGO_A_23_P148297 | |
| 335 | AGI_HUM1_OLIGO_A_23_P255785 | |
| 336 | AGI_HUM1_OLIGO_A_23_P209564 | |
| 337 | AGI_HUM1_OLIGO_A_23_P203267 | |
| 338 | AGI_HUM1_OLIGO_A_23_P55036 | |
| 339 | AGI_HUM1_OLIGO_A_23_P57306 | |
| 340 | AGI_HUM1_OLIGO_A_23_P103968 | |
| 341 | AGI_HUM1_OLIGO_A_23_P88893 | |
| 342 | AGI_HUM1_OLIGO_A_23_P139486 | |
| 343 | AGI_HUM1_OLIGO_A_23_P250164 | |
| 344 | AGI_HUM1_OLIGO_A_23_P213265 | |
| 345 | AGI_HUM1_OLIGO_A_23_P216167 | |
| 346 | AGI_HUM1_OLIGO_A_23_P153745 | |
| 347 | AGI_HUM1_OLIGO_A_23_P103614 | |
| 348 | AGI_HUM1_OLIGO_A_23_P91464 | |
| 349 | AGI_HUM1_OLIGO_A_23_P5778 | |
| 350 | AGI_HUM1_OLIGO_A_23_P251303 | |
| 351 | AGI_HUM1_OLIGO_A_23_P148807 | |
| 352 | AGI_HUM1_OLIGO_A_23_P42695 | |
| 353 | AGI_HUM1_OLIGO_A_23_P254756 | |
| 354 | AGI_HUM1_OLIGO_A_23_P103159 | |
| 355 | AGI_HUM1_OLIGO_A_23_P52121 | |
| 356 | AGI_HUM1_OLIGO_A_23_P40108 | |
| 357 | AGI_HUM1_OLIGO_A_23_P117852 | |
| 358 | AGI_HUM1_OLIGO_A_23_P48585 | |
| 359 | AGI_HUM1_OLIGO_A_23_P41796 | |
| 360 | AGI_HUM1_OLIGO_A_23_P21128 | AGI_HUM1_OLIGO_A_23_P65278 |
| 361 | AGI_HUM1_OLIGO_A_23_P151525 | |
| 362 | AGI_HUM1_OLIGO_A_23_P143694 | |
| 363 | AGI_HUM1_OLIGO_A_23_P37205 | |
| 364 | AGI_HUM1_OLIGO_A_23_P126648 | |
| 365 | AGI_HUM1_OLIGO_A_23_P2423 | |
| 366 | AGI_HUM1_OLIGO_A_23_P144348 | |
| 367 | AGI_HUM1_OLIGO_A_23_P48669 | |
| 368 | AGI_HUM1_OLIGO_A_23_P63789 | |
| 369 | AGI_HUM1_OLIGO_A_23_P129157 | |
| 370 | AGI_HUM1_OLIGO_A_23_P65307 | |
| 371 | AGI_HUM1_OLIGO_A_23_P401 | |
| 372 | AGI_HUM1_OLIGO_A_23_P82361 | |
| 373 | AGI_HUM1_OLIGO_A_23_P121064 | |
| 374 | AGI_HUM1_OLIGO_A_23_P53276 | |
| 375 | AGI_HUM1_OLIGO_A_23_P148475 | |
| 376 | AGI_HUM1_OLIGO_A_23_P209200 | |
| 377 | AGI_HUM1_OLIGO_A_23_P13694 | |
| 378 | AGI_HUM1_OLIGO_A_23_P87192 | |
| 379 | AGI_HUM1_OLIGO_A_23_P134139 | |
| 380 | AGI_HUM1_OLIGO_A_23_P215875 | |
| 381 | AGI_HUM1_OLIGO_A_23_P119562 | |
| 382 | AGI_HUM1_OLIGO_A_23_P129204 | |
| 383 | AGI_HUM1_OLIGO_A_23_P254702 | |
| 384 | AGI_HUM1_OLIGO_A_23_P41049 | |
| 385 | AGI_HUM1_OLIGO_A_23_P109143 | |
| 386 | AGI_HUM1_OLIGO_A_23_P56734 | |
| 387 | AGI_HUM1_OLIGO_A_23_P216259 | |
| 388 | AGI_HUM1_OLIGO_A_23_P134085 | |
| 389 | AGI_HUM1_OLIGO_A_23_P251825 | |
| 390 | AGI_HUM1_OLIGO_A_23_P114883 | |
| 391 | AGI_HUM1_OLIGO_A_23_P162481 | |
| 392 | AGI_HUM1_OLIGO_A_23_P121499 | |
| 393 | AGI_HUM1_OLIGO_A_23_P37892 | |
| 394 | AGI_HUM1_OLIGO_A_23_P216325 | |
| 395 | AGI_HUM1_OLIGO_A_23_P46812 | |
| 396 | AGI_HUM1_OLIGO_A_23_P12686 | |
| 397 | AGI_HUM1_OLIGO_A_23_P32707 | |
| 398 | AGI_HUM1_OLIGO_A_23_P11862 | |
| 399 | AGI_HUM1_OLIGO_A_23_P811 | |
| 400 | AGI_HUM1_OLIGO_A_23_P11543 | |
| 401 | AGI_HUM1_OLIGO_A_23_P49972 | |
| 402 | AGI_HUM1_OLIGO_A_23_P134910 | |
| 403 | AGI_HUM1_OLIGO_A_23_P53476 | |
| 404 | AGI_HUM1_OLIGO_A_23_P144096 | |
| 405 | AGI_HUM1_OLIGO_A_23_P59578 | |
| 406 | AGI_HUM1_OLIGO_A_23_P13907 | |
| 407 | AGI_HUM1_OLIGO_A_23_P253958 | |
| 408 | AGI_HUM1_OLIGO_A_23_P212844 | |
| 409 | AGI_HUM1_OLIGO_A_23_P128183 | |
| 410 | AGI_HUM1_OLIGO_A_23_P103201 | |
| 411 | AGI_HUM1_OLIGO_A_23_P155348 | |
| 412 | AGI_HUM1_OLIGO_A_23_P99292 | |
| 413 | AGI_HUM1_OLIGO_A_23_P132718 | |
| 414 | AGI_HUM1_OLIGO_A_23_P115605 | |
| 415 | AGI_HUM1_OLIGO_A_23_P54681 | |
| 416 | AGI_HUM1_OLIGO_A_23_P20463 | |
| 417 | AGI_HUM1_OLIGO_A_23_P200685 | |
| 418 | AGI_HUM1_OLIGO_A_23_P214907 | |
| 419 | AGI_HUM1_OLIGO_A_23_P102471 | |
| 420 | AGI_HUM1_OLIGO_A_23_P207911 | |
| 421 | AGI_HUM1_OLIGO_A_23_P102582 | |
| 422 | AGI_HUM1_OLIGO_A_23_P77142 | |
| 423 | AGI_HUM1_OLIGO_A_23_P122304 | |
| 424 | AGI_HUM1_OLIGO_A_23_P53456 | |
| 425 | AGI_HUM1_OLIGO_A_23_P9779 | |
| 426 | AGI_HUM1_OLIGO_A_23_P14683 | |
| 427 | AGI_HUM1_OLIGO_A_23_P106682 | |
| 428 | AGI_HUM1_OLIGO_A_23_P200414 | |
| 429 | AGI_HUM1_OLIGO_A_23_P93690 | |
| 430 | AGI_HUM1_OLIGO_A_23_P31873 | |
| 431 | AGI_HUM1_OLIGO_A_23_P55706 | |
| 432 | AGI_HUM1_OLIGO_A_23_P206544 | |
| 433 | AGI_HUM1_OLIGO_A_23_P65617 | |
| 434 | AGI_HUM1_OLIGO_A_23_P85682 | |
| 435 | AGI_HUM1_OLIGO_A_23_P24774 | |
| 436 | AGI_HUM1_OLIGO_A_23_P64617 | |
| 437 | AGI_HUM1_OLIGO_A_23_P251551 | |
| 438 | AGI_HUM1_OLIGO_A_23_P152984 | |
| 439 | AGI_HUM1_OLIGO_A_23_P154279 | |
| 440 | AGI_HUM1_OLIGO_A_23_P205580 | |
| 441 | AGI_HUM1_OLIGO_A_23_P258493 | |
| 442 | AGI_HUM1_OLIGO_A_23_P28263 | |
| 443 | AGI_HUM1_OLIGO_A_23_P94174 | |
| 444 | AGI_HUM1_OLIGO_A_23_P127394 | AGI_HUM1_OLIGO_A_23_P138787 |
| 445 | AGI_HUM1_OLIGO_A_23_P201758 | |
| 446 | AGI_HUM1_OLIGO_A_23_P150935 | |
| 447 | AGI_HUM1_OLIGO_A_23_P96590 | |
| 448 | AGI_HUM1_OLIGO_A_23_P107911 | |
| 449 | AGI_HUM1_OLIGO_A_23_P2705 | |
| 450 | AGI_HUM1_OLIGO_A_23_P162916 | |
| 451 | AGI_HUM1_OLIGO_A_23_P231 | |
| 452 | AGI_HUM1_OLIGO_A_23_P213247 | |
| 453 | AGI_HUM1_OLIGO_A_23_P252369 | |
| 454 | AGI_HUM1_OLIGO_A_23_P250801 | |
| 455 | AGI_HUM1_OLIGO_A_23_P205355 | |
| 456 | AGI_HUM1_OLIGO_A_23_P64038 | |
| 457 | AGI_HUM1_OLIGO_A_23_P32196 | |
| 458 | AGI_HUM1_OLIGO_A_23_P74290 | |
| 459 | AGI_HUM1_OLIGO_A_23_P40280 | |
| 460 | AGI_HUM1_OLIGO_A_23_P26243 | |
| 461 | AGI_HUM1_OLIGO_A_23_P39125 | |
| 462 | AGI_HUM1_OLIGO_A_23_P110253 | |
| 463 | AGI_HUM1_OLIGO_A_23_P25293 | |
| 464 | AGI_HUM1_OLIGO_A_23_P11283 | |
| 465 | AGI_HUM1_OLIGO_A_23_P21033 | |
| 466 | AGI_HUM1_OLIGO_A_23_P152415 | |
| 467 | AGI_HUM1_OLIGO_A_23_P118246 | |
| 468 | AGI_HUM1_OLIGO_A_23_P133470 | |
| 469 | AGI_HUM1_OLIGO_A_23_P112159 | |
| 470 | AGI_HUM1_OLIGO_A_23_P48570 | |
| 471 | AGI_HUM1_OLIGO_A_23_P207481 | |
| 472 | AGI_HUM1_OLIGO_A_23_P71270 | |
| 473 | AGI_HUM1_OLIGO_A_23_P17503 | |
| 474 | AGI_HUM1_OLIGO_A_23_P143348 | |
| 475 | AGI_HUM1_OLIGO_A_23_P256204 | |
| 476 | AGI_HUM1_OLIGO_A_23_P800 | |
| 477 | AGI_HUM1_OLIGO_A_23_P43226 | |
| 478 | AGI_HUM1_OLIGO_A_23_P43817 | |
| 479 | AGI_HUM1_OLIGO_A_23_P218040 | |
| 480 | AGI_HUM1_OLIGO_A_23_P107283 | |
| 481 | AGI_HUM1_OLIGO_A_23_P4462 | |
| 482 | AGI_HUM1_OLIGO_A_23_P110571 | |
| 483 | AGI_HUM1_OLIGO_A_23_P121602 | |
| 484 | AGI_HUM1_OLIGO_A_23_P154235 | |
| 485 | AGI_HUM1_OLIGO_A_23_P134854 | |
| 486 | AGI_HUM1_OLIGO_A_23_P205910 | |
| 487 | AGI_HUM1_OLIGO_A_23_P97700 | |
| 488 | AGI_HUM1_OLIGO_A_23_P255837 | |
| 489 | AGI_HUM1_OLIGO_A_23_P215976 | |
| 490 | AGI_HUM1_OLIGO_A_23_P53797 | |
| 491 | AGI_HUM1_OLIGO_A_23_P213369 | |
| 492 | AGI_HUM1_OLIGO_A_23_P132669 | |
| 493 | AGI_HUM1_OLIGO_A_23_P164283 | |
| 494 | AGI_HUM1_OLIGO_A_23_P52101 | |
| 495 | AGI_HUM1_OLIGO_A_23_P147345 | AGI_HUM1_OLIGO_A_23_P158725 |
| 496 | AGI_HUM1_OLIGO_A_23_P18684 | |
| 497 | AGI_HUM1_OLIGO_A_23_P28953 | |
| 498 | AGI_HUM1_OLIGO_A_23_P94380 | |
| 499 | AGI_HUM1_OLIGO_A_23_P202810 | |
| 500 | AGI_HUM1_OLIGO_A_23_P55769 | |
| 501 | AGI_HUM1_OLIGO_A_23_P151995 | |
| 502 | AGI_HUM1_OLIGO_A_23_P145096 | |
| 503 | AGI_HUM1_OLIGO_A_23_P64018 | |
| 504 | AGI_HUM1_OLIGO_A_23_P62890 | |
| 505 | AGI_HUM1_OLIGO_A_23_P132277 | |
| 506 | AGI_HUM1_OLIGO_A_23_P94358 | |
| 507 | AGI_HUM1_OLIGO_A_23_P47991 | |
| 508 | AGI_HUM1_OLIGO_A_23_P206228 | |
| 509 | AGI_HUM1_OLIGO_A_23_P30728 | |
| 510 | AGI_HUM1_OLIGO_A_23_P251992 | |
| 511 | AGI_HUM1_OLIGO_A_23_P159952 | |
| 512 | AGI_HUM1_OLIGO_A_23_P134755 | |
| 513 | AGI_HUM1_OLIGO_A_23_P99802 | |
| 514 | AGI_HUM1_OLIGO_A_23_P80902 | |
| 515 | AGI_HUM1_OLIGO_A_23_P150379 | |
| 516 | AGI_HUM1_OLIGO_A_23_P211180 | |
| 517 | AGI_HUM1_OLIGO_A_23_P166627 | |
| 518 | AGI_HUM1_OLIGO_A_23_P88069 | |
| 519 | AGI_HUM1_OLIGO_A_23_P128674 | |
| 520 | AGI_HUM1_OLIGO_A_23_P69121 | |
| 521 | AGI_HUM1_OLIGO_A_23_P128195 | |
| 522 | AGI_HUM1_OLIGO_A_23_P106192 | |
| 523 | AGI_HUM1_OLIGO_A_23_P163306 | |
| 524 | AGI_HUM1_OLIGO_A_23_P160214 | |
| 525 | AGI_HUM1_OLIGO_A_23_P42432 | |
| 526 | AGI_HUM1_OLIGO_A_23_P41327 | |
| 527 | AGI_HUM1_OLIGO_A_23_P120667 | |
| 528 | AGI_HUM1_OLIGO_A_23_P145194 | |
| 529 | AGI_HUM1_OLIGO_A_23_P143845 | |
| 530 | AGI_HUM1_OLIGO_A_23_P128520 | |
| 531 | AGI_HUM1_OLIGO_A_23_P14184 | |
| 532 | AGI_HUM1_OLIGO_A_23_P200118 | |
| 533 | AGI_HUM1_OLIGO_A_23_P100653 | |
| 534 | AGI_HUM1_OLIGO_A_23_P18887 | |
| 535 | AGI_HUM1_OLIGO_A_23_P87769 | |
| 536 | AGI_HUM1_OLIGO_A_23_P169277 | |
| 537 | AGI_HUM1_OLIGO_A_23_P161686 | |
| 538 | AGI_HUM1_OLIGO_A_23_P130694 | |
| 539 | AGI_HUM1_OLIGO_A_23_P137035 | |
| 540 | AGI_HUM1_OLIGO_A_23_P121926 | |
| 541 | AGI_HUM1_OLIGO_A_23_P134454 | |
| 542 | AGI_HUM1_OLIGO_A_23_P22682 | |
| 543 | AGI_HUM1_OLIGO_A_23_P25229 | |
| 544 | AGI_HUM1_OLIGO_A_23_P155857 | |
| 545 | AGI_HUM1_OLIGO_A_23_P155257 | |
| 546 | AGI_HUM1_OLIGO_A_23_P104804 | |
| 547 | AGI_HUM1_OLIGO_A_23_P93562 | |
| 548 | AGI_HUM1_OLIGO_A_23_P116202 | |
| 549 | AGI_HUM1_OLIGO_A_23_P54834 | |
| 550 | AGI_HUM1_OLIGO_A_23_P250372 | |
| 551 | AGI_HUM1_OLIGO_A_23_P205389 | |
| 552 | AGI_HUM1_OLIGO_A_23_P201747 | |
| 553 | AGI_HUM1_OLIGO_A_23_P135576 | AGI_HUM1_OLIGO_A_23_P146970 |
| 554 | AGI_HUM1_OLIGO_A_23_P57836 | |
| 555 | AGI_HUM1_OLIGO_A_23_P208880 | |
| 556 | AGI_HUM1_OLIGO_A_23_P29655 | |
| 557 | AGI_HUM1_OLIGO_A_23_P130376 | |
| 558 | AGI_HUM1_OLIGO_A_23_P111701 | |
| 559 | AGI_HUM1_OLIGO_A_23_P14754 | |
| 560 | AGI_HUM1_OLIGO_A_23_P102058 | |
| 561 | AGI_HUM1_OLIGO_A_23_P145074 | |
| 562 | AGI_HUM1_OLIGO_A_23_P91912 | |
| 563 | AGI_HUM1_OLIGO_A_23_P2041 | |
| 564 | AGI_HUM1_OLIGO_A_23_P118834 | |
| 565 | AGI_HUM1_OLIGO_A_23_P82990 | |
| 566 | AGI_HUM1_OLIGO_A_23_P118633 | |
| 567 | AGI_HUM1_OLIGO_A_23_P44139 | AGI_HUM1_OLIGO_A_23_P61009 |
| 568 | AGI_HUM1_OLIGO_A_23_P68007 | AGI_HUM1_OLIGO_A_23_P6951 |
| 569 | AGI_HUM1_OLIGO_A_23_P166640 | |
| 570 | AGI_HUM1_OLIGO_A_23_P123343 | |
| 571 | AGI_HUM1_OLIGO_A_23_P142598 | |
| 572 | AGI_HUM1_OLIGO_A_23_P108862 | |
| 573 | AGI_HUM1_OLIGO_A_23_P54165 | |
| 574 | AGI_HUM1_OLIGO_A_23_P35427 | AGI_HUM1_OLIGO_A_23_P46819 |
| 575 | AGI_HUM1_OLIGO_A_23_P3193 | |
| 576 | AGI_HUM1_OLIGO_A_23_P114670 | |
| 577 | AGI_HUM1_OLIGO_A_23_P112493 | |
| 578 | AGI_HUM1_OLIGO_A_23_P122007 | |
| 579 | AGI_HUM1_OLIGO_A_23_P253002 | |
| 580 | AGI_HUM1_OLIGO_A_23_P171249 | |
| 581 | AGI_HUM1_OLIGO_A_23_P44724 | |
| 582 | AGI_HUM1_OLIGO_A_23_P105747 | |
| 583 | AGI_HUM1_OLIGO_A_23_P160466 | |
| 584 | AGI_HUM1_OLIGO_A_23_P251945 | |
| 585 | AGI_HUM1_OLIGO_A_23_P153475 | |
| 586 | AGI_HUM1_OLIGO_A_23_P213745 | |
| 587 | AGI_HUM1_OLIGO_A_23_P146506 | |
| 588 | AGI_HUM1_OLIGO_A_23_P59261 | |
| 589 | AGI_HUM1_OLIGO_A_23_P28886 | |
| 590 | AGI_HUM1_OLIGO_A_23_P256425 | |
| 591 | AGI_HUM1_OLIGO_A_23_P134764 | |
| 592 | AGI_HUM1_OLIGO_A_23_P138417 | |
| 593 | AGI_HUM1_OLIGO_A_23_P149388 | |
| 594 | AGI_HUM1_OLIGO_A_23_P213458 | |
| 595 | AGI_HUM1_OLIGO_A_23_P88439 | |
| 596 | AGI_HUM1_OLIGO_A_23_P102202 | |
| 597 | AGI_HUM1_OLIGO_A_23_P201988 | |
| 598 | AGI_HUM1_OLIGO_A_23_P202737 | |
| 599 | AGI_HUM1_OLIGO_A_23_P19389 | |
| 600 | AGI_HUM1_OLIGO_A_23_P154488 | |
| 601 | AGI_HUM1_OLIGO_A_23_P257457 | |
| 602 | AGI_HUM1_OLIGO_A_23_P200670 | |
| 603 | AGI_HUM1_OLIGO_A_23_P40174 | |
| 604 | AGI_HUM1_OLIGO_A_23_P83857 | AGI_HUM1_OLIGO_A_23_P96410 |
| 605 | AGI_HUM1_OLIGO_A_23_P71946 | |
| 606 | AGI_HUM1_OLIGO_A_23_P92954 | |
| 607 | AGI_HUM1_OLIGO_A_23_P137196 | |
| 608 | AGI_HUM1_OLIGO_A_23_P159011 | AGI_HUM1_OLIGO_A_23_P41634 |
| 609 | AGI_HUM1_OLIGO_A_23_P150016 | |
| 610 | AGI_HUM1_OLIGO_A_23_P46928 | |
| 611 | AGI_HUM1_OLIGO_A_23_P113777 | |
| 612 | AGI_HUM1_OLIGO_A_23_P89941 | |
| 613 | AGI_HUM1_OLIGO_A_23_P124619 | |
| 614 | AGI_HUM1_OLIGO_A_23_P202232 | |
| 615 | AGI_HUM1_OLIGO_A_23_P70617 | |
| 616 | AGI_HUM1_OLIGO_A_23_P211957 | |
| 617 | AGI_HUM1_OLIGO_A_23_P57588 | |
| 618 | AGI_HUM1_OLIGO_A_23_P87049 | |
| 619 | AGI_HUM1_OLIGO_A_23_P60393 | |
| 620 | AGI_HUM1_OLIGO_A_23_P204840 | |
| 621 | AGI_HUM1_OLIGO_A_23_P154745 | |
| 622 | AGI_HUM1_OLIGO_A_23_P112026 | |
| 623 | AGI_HUM1_OLIGO_A_23_P170587 | |
| 624 | AGI_HUM1_OLIGO_A_23_P14174 | |
| 625 | AGI_HUM1_OLIGO_A_23_P35045 | |
| 626 | AGI_HUM1_OLIGO_A_23_P108574 | |
| 627 | AGI_HUM1_OLIGO_A_23_P70249 | |
| 628 | AGI_HUM1_OLIGO_A_23_P63189 | |
| 629 | AGI_HUM1_OLIGO_A_23_P114457 | |
| 630 | AGI_HUM1_OLIGO_A_23_P68717 | |
| 631 | AGI_HUM1_OLIGO_A_23_P19134 | |
| 632 | AGI_HUM1_OLIGO_A_23_P22625 | |
| 633 | AGI_HUM1_OLIGO_A_23_P2884 | |
| 634 | AGI_HUM1_OLIGO_A_23_P43800 | |
| 635 | AGI_HUM1_OLIGO_A_23_P201445 | |
| 636 | AGI_HUM1_OLIGO_A_23_P25964 | |
| 637 | AGI_HUM1_OLIGO_A_23_P171074 | |
| 638 | AGI_HUM1_OLIGO_A_23_P252292 | |
| 639 | AGI_HUM1_OLIGO_A_23_P150789 | |
| 640 | AGI_HUM1_OLIGO_A_23_P164179 | |
| 641 | AGI_HUM1_OLIGO_A_23_P200780 | |
| 642 | AGI_HUM1_OLIGO_A_23_P70007 | |
| 643 | AGI_HUM1_OLIGO_A_23_P200288 | |
| 644 | AGI_HUM1_OLIGO_A_23_P103864 | |
| 645 | AGI_HUM1_OLIGO_A_23_P87500 | |
| 646 | AGI_HUM1_OLIGO_A_23_P216307 | |
| 647 | AGI_HUM1_OLIGO_A_23_P124760 | AGI_HUM1_OLIGO_A_23_P136148 |
| 648 | AGI_HUM1_OLIGO_A_23_P89020 | |
| 649 | AGI_HUM1_OLIGO_A_23_P139934 | |
| 650 | AGI_HUM1_OLIGO_A_23_P149239 | |
| 651 | AGI_HUM1_OLIGO_A_23_P26843 | |
| 652 | AGI_HUM1_OLIGO_A_23_P128698 | |
| 653 | AGI_HUM1_OLIGO_A_23_P155968 | |
| 654 | AGI_HUM1_OLIGO_A_23_P46045 | AGI_HUM1_OLIGO_A_23_P51518 |
| 655 | AGI_HUM1_OLIGO_A_23_P20423 | |
| 656 | AGI_HUM1_OLIGO_A_23_P105264 | |
| 657 | AGI_HUM1_OLIGO_A_23_P150555 | |
| 658 | AGI_HUM1_OLIGO_A_23_P99924 | |
| 659 | AGI_HUM1_OLIGO_A_23_P112412 | |
| 660 | AGI_HUM1_OLIGO_A_23_P127128 | |
| 661 | AGI_HUM1_OLIGO_A_23_P161352 | |
| 662 | AGI_HUM1_OLIGO_A_23_P11136 | |
| 663 | AGI_HUM1_OLIGO_A_23_P326319 | |
| 664 | AGI_HUM1_OLIGO_A_23_P206776 | |
| 665 | AGI_HUM1_OLIGO_A_23_P48997 | |
| 666 | AGI_HUM1_OLIGO_A_23_P216448 | |
| 667 | AGI_HUM1_OLIGO_A_23_P145 | |
| 668 | AGI_HUM1_OLIGO_A_23_P211985 | |
| 669 | AGI_HUM1_OLIGO_A_23_P91590 | |
| 670 | AGI_HUM1_OLIGO_A_23_P24244 | |
| 671 | AGI_HUM1_OLIGO_A_23_P41227 | |
| 672 | AGI_HUM1_OLIGO_A_23_P145895 | |
| 673 | AGI_HUM1_OLIGO_A_23_P254271 | |
| 674 | AGI_HUM1_OLIGO_A_23_P120105 | |
| 675 | AGI_HUM1_OLIGO_A_23_P52797 | |
| 676 | AGI_HUM1_OLIGO_A_23_P14273 | |
| 677 | AGI_HUM1_OLIGO_A_23_P120315 | |
| 678 | AGI_HUM1_OLIGO_A_23_P35114 | |
| 679 | AGI_HUM1_OLIGO_A_23_P205200 | |
| 680 | AGI_HUM1_OLIGO_A_23_P115759 | |
| 681 | AGI_HUM1_OLIGO_A_23_P213728 | |
| 682 | AGI_HUM1_OLIGO_A_23_P113161 | |
| 683 | AGI_HUM1_OLIGO_A_23_P161152 | |
| 684 | AGI_HUM1_OLIGO_A_23_P59926 | |
| 685 | AGI_HUM1_OLIGO_A_23_P160159 | |
| 686 | AGI_HUM1_OLIGO_A_23_P138686 | |
| 687 | AGI_HUM1_OLIGO_A_23_P57658 | |
| 688 | AGI_HUM1_OLIGO_A_23_P130182 | |
| 689 | AGI_HUM1_OLIGO_A_23_P216004 | |
| 690 | AGI_HUM1_OLIGO_A_23_P129824 | AGI_HUM1_OLIGO_A_23_P38190 |
| 691 | AGI_HUM1_OLIGO_A_23_P8241 | |
| 692 | AGI_HUM1_OLIGO_A_23_P40989 | |
| 693 | AGI_HUM1_OLIGO_A_23_P15146 | |
| 694 | AGI_HUM1_OLIGO_A_23_P54276 | |
| 695 | AGI_HUM1_OLIGO_A_23_P117882 | |
| 696 | AGI_HUM1_OLIGO_A_23_P43019 | |
| 697 | AGI_HUM1_OLIGO_A_23_P89056 | |
| 698 | AGI_HUM1_OLIGO_A_23_P168747 | |
| 699 | AGI_HUM1_OLIGO_A_23_P210726 | |
| 700 | AGI_HUM1_OLIGO_A_23_P137731 | |
| 701 | AGI_HUM1_OLIGO_A_23_P20683 | |
| 702 | AGI_HUM1_OLIGO_A_23_P256033 | |
| 703 | AGI_HUM1_OLIGO_A_23_P159053 | |
| 704 | AGI_HUM1_OLIGO_A_23_P251095 | |
| 705 | AGI_HUM1_OLIGO_A_23_P209946 | |
| 706 | AGI_HUM1_OLIGO_A_23_P208896 | |
| 707 | AGI_HUM1_OLIGO_A_23_P106727 | |
| 708 | AGI_HUM1_OLIGO_A_23_P133058 | |
| 709 | AGI_HUM1_OLIGO_A_23_P3111 | |
| 710 | AGI_HUM1_OLIGO_A_23_P98115 | |
| 711 | AGI_HUM1_OLIGO_A_23_P160518 | |
| 712 | AGI_HUM1_OLIGO_A_23_P79628 | |
| 713 | AGI_HUM1_OLIGO_A_23_P7596 | |
| 714 | AGI_HUM1_OLIGO_A_23_P66798 | |
| 715 | AGI_HUM1_OLIGO_A_23_P200507 | |
| 716 | AGI_HUM1_OLIGO_A_23_P148879 | |
| 717 | AGI_HUM1_OLIGO_A_23_P86283 | |
| 718 | AGI_HUM1_OLIGO_A_23_P13065 | |
| 719 | AGI_HUM1_OLIGO_A_23_P18325 | |
| 720 | AGI_HUM1_OLIGO_A_23_P155765 | |
| 721 | AGI_HUM1_OLIGO_A_23_P71017 | |
| 722 | AGI_HUM1_OLIGO_A_23_P214244 | |
| 723 | AGI_HUM1_OLIGO_A_23_P93543 | |
| 724 | AGI_HUM1_OLIGO_A_23_P169576 | |
| 725 | AGI_HUM1_OLIGO_A_23_P101410 | |
| 726 | AGI_HUM1_OLIGO_A_23_P82478 | |
| 727 | AGI_HUM1_OLIGO_A_23_P64010 | |
| 728 | AGI_HUM1_OLIGO_A_23_P257131 | |
| 729 | AGI_HUM1_OLIGO_A_23_P216454 | |
| 730 | AGI_HUM1_OLIGO_A_23_P208500 | |
| 731 | AGI_HUM1_OLIGO_A_23_P59005 | |
| 732 | AGI_HUM1_OLIGO_A_23_P217998 | |
| 733 | AGI_HUM1_OLIGO_A_23_P70708 | |
| 734 | AGI_HUM1_OLIGO_A_23_P80438 | |
| 735 | AGI_HUM1_OLIGO_A_23_P214080 | |
| 736 | AGI_HUM1_OLIGO_A_23_P69109 | |
| 737 | AGI_HUM1_OLIGO_A_23_P215517 | |
| 738 | AGI_HUM1_OLIGO_A_23_P62807 | |
| 739 | AGI_HUM1_OLIGO_A_23_P89780 | |
| 740 | AGI_HUM1_OLIGO_A_23_P82748 | |
| 741 | AGI_HUM1_OLIGO_A_23_P103223 | |
| 742 | AGI_HUM1_OLIGO_A_23_P139919 | |
| 743 | AGI_HUM1_OLIGO_A_23_P27795 | |
| 744 | AGI_HUM1_OLIGO_A_23_P63649 | |
| 745 | AGI_HUM1_OLIGO_A_23_P17808 | |
| 746 | AGI_HUM1_OLIGO_A_23_P161474 | |
| 747 | AGI_HUM1_OLIGO_A_23_P27133 | |
| 748 | AGI_HUM1_OLIGO_A_23_P16944 | |
| 749 | AGI_HUM1_OLIGO_A_23_P207507 | |
| 750 | AGI_HUM1_OLIGO_A_23_P4649 | |
| 751 | AGI_HUM1_OLIGO_A_23_P113078 | AGI_HUM1_OLIGO_A_23_P124470 |
| 752 | AGI_HUM1_OLIGO_A_23_P128323 | |
| 753 | AGI_HUM1_OLIGO_A_23_P20494 | |
| 754 | AGI_HUM1_OLIGO_A_23_P149615 | |
| 755 | AGI_HUM1_OLIGO_A_23_P251701 | |
| 756 | AGI_HUM1_OLIGO_A_23_P124946 | |
| 757 | AGI_HUM1_OLIGO_A_23_P32758 | AGI_HUM1_OLIGO_A_23_P94795 |
| 758 | AGI_HUM1_OLIGO_A_23_P75310 | |
| 759 | AGI_HUM1_OLIGO_A_23_P149019 | |
| 760 | AGI_HUM1_OLIGO_A_23_P26756 | |
| 761 | AGI_HUM1_OLIGO_A_23_P207957 | |
| 762 | AGI_HUM1_OLIGO_A_23_P134953 | |
| 763 | AGI_HUM1_OLIGO_A_23_P97632 | |
| 764 | AGI_HUM1_OLIGO_A_23_P202587 | |
| 765 | AGI_HUM1_OLIGO_A_23_P95810 | |
| 766 | AGI_HUM1_OLIGO_A_23_P250212 | |
| 767 | AGI_HUM1_OLIGO_A_23_P48339 | |
| 768 | AGI_HUM1_OLIGO_A_23_P146584 | |
| 769 | AGI_HUM1_OLIGO_A_23_P115366 | |
| 770 | AGI_HUM1_OLIGO_A_23_P166663 | |
| 771 | AGI_HUM1_OLIGO_A_23_P255884 | |
| 772 | AGI_HUM1_OLIGO_A_23_P12133 | |
| 773 | AGI_HUM1_OLIGO_A_23_P32558 | |
| 774 | AGI_HUM1_OLIGO_A_23_P142389 | |
| 775 | AGI_HUM1_OLIGO_A_23_P99172 | |
| 776 | AGI_HUM1_OLIGO_A_23_P49768 | |
| 777 | AGI_HUM1_OLIGO_A_23_P213910 | |
| 778 | AGI_HUM1_OLIGO_A_23_P213093 | |
| 779 | AGI_HUM1_OLIGO_A_23_P208991 | |
| 780 | AGI_HUM1_OLIGO_A_23_P117037 | |
| 781 | AGI_HUM1_OLIGO_A_23_P257863 | |
| 782 | AGI_HUM1_OLIGO_A_23_P250462 | |
| 783 | AGI_HUM1_OLIGO_A_23_P156898 | |
| 784 | AGI_HUM1_OLIGO_A_23_P212968 | |
| 785 | AGI_HUM1_OLIGO_A_23_P117602 | |
| 786 | AGI_HUM1_OLIGO_A_23_P200653 | |
| 787 | AGI_HUM1_OLIGO_A_23_P54626 | |
| 788 | AGI_HUM1_OLIGO_A_23_P201576 | |
| 789 | AGI_HUM1_OLIGO_A_23_P213986 | AGI_HUM1_OLIGO_A_23_P28772 |
| 790 | AGI_HUM1_OLIGO_A_23_P31713 | |
| 791 | AGI_HUM1_OLIGO_A_23_P6263 | |
| 792 | AGI_HUM1_OLIGO_A_23_P203458 | |
| 793 | AGI_HUM1_OLIGO_A_23_P51906 | |
| 794 | AGI_HUM1_OLIGO_A_23_P43334 | |
| 795 | AGI_HUM1_OLIGO_A_23_P159027 | |
| 796 | AGI_HUM1_OLIGO_A_23_P76109 | |
| 797 | AGI_HUM1_OLIGO_A_23_P105794 | |
| 798 | AGI_HUM1_OLIGO_A_23_P127579 | |
| 799 | AGI_HUM1_OLIGO_A_23_P253524 | |
| 800 | AGI_HUM1_OLIGO_A_23_P42220 | |
| 801 | AGI_HUM1_OLIGO_A_23_P58036 | |
| 802 | AGI_HUM1_OLIGO_A_23_P30254 | |
| 803 | AGI_HUM1_OLIGO_A_23_P51231 | |
| 804 | AGI_HUM1_OLIGO_A_23_P165051 | |
| 805 | AGI_HUM1_OLIGO_A_23_P165937 | |
| 806 | AGI_HUM1_OLIGO_A_23_P215956 | |
| 807 | AGI_HUM1_OLIGO_A_23_P60028 | |
| 808 | AGI_HUM1_OLIGO_A_23_P62830 | |
| 809 | AGI_HUM1_OLIGO_A_23_P155358 | |
| 810 | AGI_HUM1_OLIGO_A_23_P37727 | |
| 811 | AGI_HUM1_OLIGO_A_23_P71290 | |
| 812 | AGI_HUM1_OLIGO_A_23_P122915 | |
| 813 | AGI_HUM1_OLIGO_A_23_P209298 | |
| 814 | AGI_HUM1_OLIGO_A_23_P216340 | |
| 815 | AGI_HUM1_OLIGO_A_23_P203856 | |
| 816 | AGI_HUM1_OLIGO_A_23_P30655 | |
| 817 | AGI_HUM1_OLIGO_A_23_P52058 | |
| 818 | AGI_HUM1_OLIGO_A_23_P37111 | |
| 819 | AGI_HUM1_OLIGO_A_23_P13382 | |
| 820 | AGI_HUM1_OLIGO_A_23_P166899 | |
| 821 | AGI_HUM1_OLIGO_A_23_P137143 | |
| 822 | AGI_HUM1_OLIGO_A_23_P120054 | |
| 823 | AGI_HUM1_OLIGO_A_23_P21017 | |
| 824 | AGI_HUM1_OLIGO_A_23_P113582 | AGI_HUM1_OLIGO_A_23_P250571 |
| 825 | AGI_HUM1_OLIGO_A_23_P133424 | |
| 826 | AGI_HUM1_OLIGO_A_23_P6433 | |
| 827 | AGI_HUM1_OLIGO_A_23_P49279 | |
| 828 | AGI_HUM1_OLIGO_A_23_P152755 | |
| 829 | AGI_HUM1_OLIGO_A_23_P73239 | |
| 830 | AGI_HUM1_OLIGO_A_23_P44974 | |
| 831 | AGI_HUM1_OLIGO_A_23_P162579 | |
| 832 | AGI_HUM1_OLIGO_A_23_P88522 | |
| 833 | AGI_HUM1_OLIGO_A_23_P115732 | |
| 834 | AGI_HUM1_OLIGO_A_23_P85703 | |
| 835 | AGI_HUM1_OLIGO_A_23_P79681 | |
| 836 | AGI_HUM1_OLIGO_A_23_P14886 | |
| 837 | AGI_HUM1_OLIGO_A_23_P29663 | |
| 838 | AGI_HUM1_OLIGO_A_23_P121326 | |
| 839 | AGI_HUM1_OLIGO_A_23_P215607 | |
| 840 | AGI_HUM1_OLIGO_A_23_P85800 | |
| 841 | AGI_HUM1_OLIGO_A_23_P149111 | |
| 842 | AGI_HUM1_OLIGO_A_23_P55936 | |
| 843 | AGI_HUM1_OLIGO_A_23_P36464 | |
| 844 | AGI_HUM1_OLIGO_A_23_P21644 | |
| 845 | AGI_HUM1_OLIGO_A_23_P253345 | |
| 846 | AGI_HUM1_OLIGO_A_23_P151405 | |
| 847 | AGI_HUM1_OLIGO_A_23_P121806 | |
| 848 | AGI_HUM1_OLIGO_A_23_P156067 | |
| 849 | AGI_HUM1_OLIGO_A_23_P24433 | |
| 850 | AGI_HUM1_OLIGO_A_23_P72668 | |
| 851 | AGI_HUM1_OLIGO_A_23_P98350 | |
| 852 | AGI_HUM1_OLIGO_A_23_P76538 | |
| 853 | AGI_HUM1_OLIGO_A_23_P75780 | |
| 854 | AGI_HUM1_OLIGO_A_23_P96149 | |
| 855 | AGI_HUM1_OLIGO_A_23_P140029 | |
| 856 | AGI_HUM1_OLIGO_A_23_P48790 | |
| 857 | AGI_HUM1_OLIGO_A_23_P208866 | |
| 858 | AGI_HUM1_OLIGO_A_23_P251606 | |
| 859 | AGI_HUM1_OLIGO_A_23_P140725 | |
| 860 | AGI_HUM1_OLIGO_A_23_P31085 | |
| 861 | AGI_HUM1_OLIGO_A_23_P450035 | |
| 862 | AGI_HUM1_OLIGO_A_23_P37399 | |
| 863 | AGI_HUM1_OLIGO_A_23_P31034 | |
| 864 | AGI_HUM1_OLIGO_A_23_P101801 | |
| 865 | AGI_HUM1_OLIGO_A_23_P125519 | |
| 866 | AGI_HUM1_OLIGO_A_23_P93348 | |
| 867 | AGI_HUM1_OLIGO_A_23_P146077 | AGI_HUM1_OLIGO_A_23_P157460 |
| 868 | AGI_HUM1_OLIGO_A_23_P208779 | |
| 869 | AGI_HUM1_OLIGO_A_23_P160618 | |
| 870 | AGI_HUM1_OLIGO_A_23_P214059 | |
| 871 | AGI_HUM1_OLIGO_A_23_P13897 | |
| 872 | AGI_HUM1_OLIGO_A_23_P127426 | |
| 873 | AGI_HUM1_OLIGO_A_23_P86653 | |
| 874 | AGI_HUM1_OLIGO_A_23_P259127 | |
| 875 | AGI_HUM1_OLIGO_A_23_P63459 | |
| 876 | AGI_HUM1_OLIGO_A_23_P160537 | |
| 877 | AGI_HUM1_OLIGO_A_23_P202448 | |
| 878 | AGI_HUM1_OLIGO_A_23_P40415 | |
| 879 | AGI_HUM1_OLIGO_A_23_P154367 | |
| 880 | AGI_HUM1_OLIGO_A_23_P79259 | |
| 881 | AGI_HUM1_OLIGO_A_23_P43255 | |
| 882 | AGI_HUM1_OLIGO_A_23_P126992 | |
| 883 | AGI_HUM1_OLIGO_A_23_P258321 | |
| 884 | AGI_HUM1_OLIGO_A_23_P144465 | |
| 885 | AGI_HUM1_OLIGO_A_23_P253281 | |
| 886 | AGI_HUM1_OLIGO_A_23_P571 | |
| 887 | AGI_HUM1_OLIGO_A_23_P45928 | |
| 888 | AGI_HUM1_OLIGO_A_23_P153320 | |
| 889 | AGI_HUM1_OLIGO_A_23_P115172 | |
| 890 | AGI_HUM1_OLIGO_A_23_P138975 | |
| 891 | AGI_HUM1_OLIGO_A_23_P168846 | |
| 892 | AGI_HUM1_OLIGO_A_23_P203900 | |
| 893 | AGI_HUM1_OLIGO_A_23_P80129 | |
| 894 | AGI_HUM1_OLIGO_A_23_P73750 | |
| 895 | AGI_HUM1_OLIGO_A_23_P74320 | |
| 896 | AGI_HUM1_OLIGO_A_23_P36985 | |
| 897 | AGI_HUM1_OLIGO_A_23_P202837 | |
| 898 | AGI_HUM1_OLIGO_A_23_P28507 | |
| 899 | AGI_HUM1_OLIGO_A_23_P155332 | |
| 900 | AGI_HUM1_OLIGO_A_23_P11874 | AGI_HUM1_OLIGO_A_23_P476 |
| 901 | AGI_HUM1_OLIGO_A_23_P38677 | |
| 902 | AGI_HUM1_OLIGO_A_23_P110712 | |
| 903 | AGI_HUM1_OLIGO_A_23_P74538 | |
| 904 | AGI_HUM1_OLIGO_A_23_P98022 | |
| 905 | AGI_HUM1_OLIGO_A_23_P160503 | |
| 906 | AGI_HUM1_OLIGO_A_23_P30175 | |
| 907 | AGI_HUM1_OLIGO_A_23_P154889 | |
| 908 | AGI_HUM1_OLIGO_A_23_P218827 | |
| 909 | AGI_HUM1_OLIGO_A_23_P152687 | |
| 910 | AGI_HUM1_OLIGO_A_23_P45365 | |
| 911 | AGI_HUM1_OLIGO_A_23_P156957 | |
| 912 | AGI_HUM1_OLIGO_A_23_P167599 | |
| 913 | AGI_HUM1_OLIGO_A_23_P94103 | |
| 914 | AGI_HUM1_OLIGO_A_23_P164826 | |
| 915 | AGI_HUM1_OLIGO_A_23_P43071 | |
| 916 | AGI_HUM1_OLIGO_A_23_P64873 | |
| 917 | AGI_HUM1_OLIGO_A_23_P115022 | |
| 918 | AGI_HUM1_OLIGO_A_23_P79842 | |
| 919 | AGI_HUM1_OLIGO_A_23_P15394 | |
| 920 | AGI_HUM1_OLIGO_A_23_P58266 | |
| 921 | AGI_HUM1_OLIGO_A_23_P42331 | |
| 922 | AGI_HUM1_OLIGO_A_23_P119943 | |
| 923 | AGI_HUM1_OLIGO_A_23_P69537 | |
| 924 | AGI_HUM1_OLIGO_A_23_P213754 | |
| 925 | AGI_HUM1_OLIGO_A_23_P77328 | |
| 926 | AGI_HUM1_OLIGO_A_23_P89455 | |
| 927 | AGI_HUM1_OLIGO_A_23_P160148 | |
| 928 | AGI_HUM1_OLIGO_A_23_P160807 | |
| 929 | AGI_HUM1_OLIGO_A_23_P56630 | |
| 930 | AGI_HUM1_OLIGO_A_23_P76914 | |
| 931 | AGI_HUM1_OLIGO_A_23_P105571 | |
| 932 | AGI_HUM1_OLIGO_A_23_P69788 | |
| 933 | AGI_HUM1_OLIGO_A_23_P208801 | |
| 934 | AGI_HUM1_OLIGO_A_23_P206280 | |
| 935 | AGI_HUM1_OLIGO_A_23_P118384 | |
| 936 | AGI_HUM1_OLIGO_A_23_P205493 | |
| 937 | AGI_HUM1_OLIGO_A_23_P33723 | |
| 938 | AGI_HUM1_OLIGO_A_23_P22224 | |
| 939 | AGI_HUM1_OLIGO_A_23_P87150 | |
| 940 | AGI_HUM1_OLIGO_A_23_P17663 | |
| 941 | AGI_HUM1_OLIGO_A_23_P213718 | |
| 942 | AGI_HUM1_OLIGO_A_23_P154627 | |
| 943 | AGI_HUM1_OLIGO_A_23_P150092 | |
| 944 | AGI_HUM1_OLIGO_A_23_P103100 | |
| 945 | AGI_HUM1_OLIGO_A_23_P118552 | |
| 946 | AGI_HUM1_OLIGO_A_23_P147397 | |
| 947 | AGI_HUM1_OLIGO_A_23_P118451 | |
| 948 | AGI_HUM1_OLIGO_A_23_P212608 | |
| 949 | AGI_HUM1_OLIGO_A_23_P50608 | |
| 950 | AGI_HUM1_OLIGO_A_23_P137514 | |
| 951 | AGI_HUM1_OLIGO_A_23_P121716 | |
| 952 | AGI_HUM1_OLIGO_A_23_P153487 | |
| 953 | AGI_HUM1_OLIGO_A_23_P79302 | |
| 954 | AGI_HUM1_OLIGO_A_23_P56956 | |
| 955 | AGI_HUM1_OLIGO_A_23_P20648 | |
| 956 | AGI_HUM1_OLIGO_A_23_P43175 | |
| 957 | AGI_HUM1_OLIGO_A_23_P93311 | |
| 958 | AGI_HUM1_OLIGO_A_23_P10385 | AGI_HUM1_OLIGO_A_23_P84620 |
| 959 | AGI_HUM1_OLIGO_A_23_P97865 | |
| 960 | AGI_HUM1_OLIGO_A_23_P2181 | |
| 961 | AGI_HUM1_OLIGO_A_23_P252403 | |
| 962 | AGI_HUM1_OLIGO_A_23_P66137 | |
| 963 | AGI_HUM1_OLIGO_A_23_P209043 | |
| 964 | AGI_HUM1_OLIGO_A_23_P210482 | |
| 965 | AGI_HUM1_OLIGO_A_23_P64743 | |
| 966 | AGI_HUM1_OLIGO_A_23_P257031 | |
| 967 | AGI_HUM1_OLIGO_A_23_P103773 | |
| 968 | AGI_HUM1_OLIGO_A_23_P162068 | |
| 969 | AGI_HUM1_OLIGO_A_23_P65651 | |
| 970 | AGI_HUM1_OLIGO_A_23_P139965 | |
| 971 | AGI_HUM1_OLIGO_A_23_P7882 | |
| 972 | AGI_HUM1_OLIGO_A_23_P76659 | |
| 973 | AGI_HUM1_OLIGO_A_23_P210253 | |
| 974 | AGI_HUM1_OLIGO_A_23_P146548 | |
| 975 | AGI_HUM1_OLIGO_A_23_P142560 | |
| 976 | AGI_HUM1_OLIGO_A_23_P41912 | |
| 977 | AGI_HUM1_OLIGO_A_23_P17392 | |
| 978 | AGI_HUM1_OLIGO_A_23_P252371 | |
| 979 | AGI_HUM1_OLIGO_A_23_P110676 | |
| 980 | AGI_HUM1_OLIGO_A_23_P251633 | |
| 981 | AGI_HUM1_OLIGO_A_23_P156687 | |
| 982 | AGI_HUM1_OLIGO_A_23_P114232 | |
| 983 | AGI_HUM1_OLIGO_A_23_P38748 | |
| 984 | AGI_HUM1_OLIGO_A_23_P769 | |
| 985 | AGI_HUM1_OLIGO_A_23_P127789 | AGI_HUM1_OLIGO_A_23_P21363 |
| 986 | AGI_HUM1_OLIGO_A_23_P201357 | |
| 987 | AGI_HUM1_OLIGO_A_23_P256432 | |
| 988 | AGI_HUM1_OLIGO_A_23_P86470 | |
| 989 | AGI_HUM1_OLIGO_A_23_P11372 | |
| 990 | AGI_HUM1_OLIGO_A_23_P255363 | |
| 991 | AGI_HUM1_OLIGO_A_23_P205480 | |
| 992 | AGI_HUM1_OLIGO_A_23_P72897 | |
| 993 | AGI_HUM1_OLIGO_A_23_P42908 | |
| 994 | AGI_HUM1_OLIGO_A_23_P137525 | |
| 995 | AGI_HUM1_OLIGO_A_23_P214121 | |
| 996 | AGI_HUM1_OLIGO_A_23_P84510 | |
| 997 | AGI_HUM1_OLIGO_A_23_P159927 | |
| 998 | AGI_HUM1_OLIGO_A_23_P170058 | |
| 999 | AGI_HUM1_OLIGO_A_23_P202565 | |
| 1000 | AGI_HUM1_OLIGO_A_23_P158945 | AGI_HUM1_OLIGO_A_23_P170337 |
| 1001 | AGI_HUM1_OLIGO_A_23_P206369 | |
| 1002 | AGI_HUM1_OLIGO_A_23_P218086 | |
| 1003 | AGI_HUM1_OLIGO_A_23_P106405 | |
| 1004 | AGI_HUM1_OLIGO_A_23_P131475 | |
| 1005 | AGI_HUM1_OLIGO_A_23_P40885 | |
| 1006 | AGI_HUM1_OLIGO_A_23_P218988 | |
| 1007 | AGI_HUM1_OLIGO_A_23_P46141 | |
| 1008 | AGI_HUM1_OLIGO_A_23_P69293 | |
| 1009 | AGI_HUM1_OLIGO_A_23_P40209 | |
| 1010 | AGI_HUM1_OLIGO_A_23_P139575 | |
| 1011 | AGI_HUM1_OLIGO_A_23_P204850 | |
| 1012 | AGI_HUM1_OLIGO_A_23_P69249 | |
| 1013 | AGI_HUM1_OLIGO_A_23_P120325 | |
| 1014 | AGI_HUM1_OLIGO_A_23_P202852 | |
| 1015 | AGI_HUM1_OLIGO_A_23_P12100 | |
| 1016 | AGI_HUM1_OLIGO_A_23_P136460 | |
| 1017 | AGI_HUM1_OLIGO_A_23_P64611 | |
| 1018 | AGI_HUM1_OLIGO_A_23_P152087 | |
| 1019 | AGI_HUM1_OLIGO_A_23_P129034 | |
| 1020 | AGI_HUM1_OLIGO_A_23_P141965 | |
| 1021 | AGI_HUM1_OLIGO_A_23_P252711 | |
| 1022 | AGI_HUM1_OLIGO_A_23_P165896 | |
| 1023 | AGI_HUM1_OLIGO_A_23_P160188 | |
| 1024 | AGI_HUM1_OLIGO_A_23_P13364 | |
| 1025 | AGI_HUM1_OLIGO_A_23_P46084 | |
| 1026 | AGI_HUM1_OLIGO_A_23_P11685 | |
| 1027 | AGI_HUM1_OLIGO_A_23_P94533 | |
| 1028 | AGI_HUM1_OLIGO_A_23_P133684 | |
| 1029 | AGI_HUM1_OLIGO_A_23_P164170 | |
| 1030 | AGI_HUM1_OLIGO_A_23_P91095 | |
| 1031 | AGI_HUM1_OLIGO_A_23_P74914 | |
| 1032 | AGI_HUM1_OLIGO_A_23_P53439 | |
| 1033 | AGI_HUM1_OLIGO_A_23_P1691 | |
| 1034 | AGI_HUM1_OLIGO_A_23_P142714 | |
| 1035 | AGI_HUM1_OLIGO_A_23_P166246 | |
| 1036 | AGI_HUM1_OLIGO_A_23_P120883 | |
| 1037 | AGI_HUM1_OLIGO_A_23_P38876 | |
| 1038 | AGI_HUM1_OLIGO_A_23_P108785 | |
| 1039 | AGI_HUM1_OLIGO_A_23_P162047 | |
| 1040 | AGI_HUM1_OLIGO_A_23_P21382 | |
| 1041 | AGI_HUM1_OLIGO_A_23_P2366 | |
| 1042 | AGI_HUM1_OLIGO_A_23_P24984 | |
| 1043 | AGI_HUM1_OLIGO_A_23_P200203 | |
| 1044 | AGI_HUM1_OLIGO_A_23_P1461 | |
| 1045 | AGI_HUM1_OLIGO_A_23_P215320 | |
| 1046 | AGI_HUM1_OLIGO_A_23_P106204 | |
| 1047 | AGI_HUM1_OLIGO_A_23_P126057 | |
| 1048 | AGI_HUM1_OLIGO_A_23_P146497 | |
| 1049 | AGI_HUM1_OLIGO_A_23_P3146 | |
| 1050 | AGI_HUM1_OLIGO_A_23_P152160 | |
| 1051 | AGI_HUM1_OLIGO_A_23_P26976 | |
| 1052 | AGI_HUM1_OLIGO_A_23_P45756 | |
| 1053 | AGI_HUM1_OLIGO_A_23_P259166 | |
| 1054 | AGI_HUM1_OLIGO_A_23_P85067 | |
| 1055 | AGI_HUM1_OLIGO_A_23_P64129 | |
| 1056 | AGI_HUM1_OLIGO_A_23_P3204 | |
| 1057 | AGI_HUM1_OLIGO_A_23_P114947 | |
| 1058 | AGI_HUM1_OLIGO_A_23_P21560 | |
| 1059 | AGI_HUM1_OLIGO_A_23_P49033 | |
| 1060 | AGI_HUM1_OLIGO_A_23_P119196 | |
| 1061 | AGI_HUM1_OLIGO_A_23_P63829 | |
| 1062 | AGI_HUM1_OLIGO_A_23_P53891 | |
| 1063 | AGI_HUM1_OLIGO_A_23_P1552 | |
| 1064 | AGI_HUM1_OLIGO_A_23_P29723 | |
| 1065 | AGI_HUM1_OLIGO_A_23_P23855 | |
| 1066 | AGI_HUM1_OLIGO_A_23_P250833 | |
| 1067 | AGI_HUM1_OLIGO_A_23_P127676 | |
| 1068 | AGI_HUM1_OLIGO_A_23_P153351 | |
| 1069 | AGI_HUM1_OLIGO_A_23_P62948 | |
| 1070 | AGI_HUM1_OLIGO_A_23_P86899 | |
| 1071 | AGI_HUM1_OLIGO_A_23_P86012 | |
| 1072 | AGI_HUM1_OLIGO_A_23_P118543 | |
| 1073 | AGI_HUM1_OLIGO_A_23_P98092 | |
| 1074 | AGI_HUM1_OLIGO_A_23_P250063 | |
| 1075 | AGI_HUM1_OLIGO_A_23_P140364 | |
| 1076 | AGI_HUM1_OLIGO_A_23_P158239 | AGI_HUM1_OLIGO_A_23_P169629 |
| 1077 | AGI_HUM1_OLIGO_A_23_P161280 | |
| 1078 | AGI_HUM1_OLIGO_A_23_P90099 | |
| 1079 | AGI_HUM1_OLIGO_A_23_P89030 | |
| 1080 | AGI_HUM1_OLIGO_A_23_P48728 | |
| 1081 | AGI_HUM1_OLIGO_A_23_P48166 | |
| 1082 | AGI_HUM1_OLIGO_A_23_P113453 | |
| 1083 | AGI_HUM1_OLIGO_A_23_P140384 | |
| 1084 | AGI_HUM1_OLIGO_A_23_P27315 | |
| 1085 | AGI_HUM1_OLIGO_A_23_P29763 | |
| 1086 | AGI_HUM1_OLIGO_A_23_P138034 | |
| 1087 | AGI_HUM1_OLIGO_A_23_P71558 | |
| 1088 | AGI_HUM1_OLIGO_A_23_P91390 | |
| 1089 | AGI_HUM1_OLIGO_A_23_P151384 | |
| 1090 | AGI_HUM1_OLIGO_A_23_P157465 | |
| 1091 | AGI_HUM1_OLIGO_A_23_P34794 | |
| 1092 | AGI_HUM1_OLIGO_A_23_P8522 | |
| 1093 | AGI_HUM1_OLIGO_A_23_P26823 | |
| 1094 | AGI_HUM1_OLIGO_A_23_P216071 | |
| 1095 | AGI_HUM1_OLIGO_A_23_P45496 | |
| 1096 | AGI_HUM1_OLIGO_A_23_P153266 | |
| 1097 | AGI_HUM1_OLIGO_A_23_P63009 | |
| 1098 | AGI_HUM1_OLIGO_A_23_P33364 | |
| 1099 | AGI_HUM1_OLIGO_A_23_P87616 | |
| 1100 | AGI_HUM1_OLIGO_A_23_P159125 | |
| 1101 | AGI_HUM1_OLIGO_A_23_P49429 | |
| 1102 | AGI_HUM1_OLIGO_A_23_P138655 | |

| 7P-SAM-Order | ProbeID |
|---|---|
| 1 | |
| 2 | |
| 3 | |
| 4 | |
| 5 | |
| 6 | |
| 7 | |
| 8 | |
| 9 | |
| 10 | |
| 11 | |
| 12 | |
| 13 | |
| 14 | |
| 15 | |
| 16 | |
| 17 | |
| 18 | |
| 19 | |
| 20 | |
| 21 | |
| 22 | |
| 23 | |
| 24 | |
| 25 | |
| 26 | |
| 27 | |
| 28 | |
| 29 | |
| 30 | |
| 31 | |
| 32 | |
| 33 | |
| 34 | |
| 35 | |
| 36 | |
| 37 | |
| 38 | |
| 39 | |
| 40 | |
| 41 | |
| 42 | |
| 43 | |
| 44 | |
| 45 | |
| 46 | |
| 47 | |
| 48 | |
| 49 | |
| 50 | |
| 51 | |
| 52 | |
| 53 | |
| 54 | |
| 55 | |
| 56 | |
| 57 | |
| 58 | |
| 59 | |
| 60 | |
| 61 | |
| 62 | |
| 63 | |
| 64 | |
| 65 | |
| 66 | |
| 67 | |
| 68 | |
| 69 | |
| 70 | |
| 71 | |
| 72 | |
| 73 | |
| 74 | |
| 75 | |
| 76 | |
| 77 | |
| 78 | |
| 79 | |
| 80 | |
| 81 | |
| 82 | |
| 83 | |
| 84 | |
| 85 | |
| 86 | |
| 87 | |
| 88 | |
| 89 | |
| 90 | |
| 91 | |
| 92 | |
| 93 | |
| 94 | |
| 95 | |
| 96 | |
| 97 | |
| 98 | |
| 99 | |
| 100 | |
| 101 | |
| 102 | |
| 103 | |
| 104 | |
| 105 | |
| 106 | |
| 107 | |
| 108 | |
| 109 | |
| 110 | |
| 111 | |
| 112 | |
| 113 | |
| 114 | |
| 115 | |
| 116 | |
| 117 | |
| 118 | |
| 119 | |
| 120 | |
| 121 | |
| 122 | |
| 123 | |
| 124 | |
| 125 | |
| 126 | |
| 127 | |
| 128 | |
| 129 | |
| 130 | |
| 131 | |
| 132 | |
| 133 | |
| 134 | |
| 135 | |
| 136 | |
| 137 | |
| 138 | |
| 139 | |
| 140 | |
| 141 | |
| 142 | |
| 143 | |
| 144 | |
| 145 | |
| 146 | |
| 147 | |
| 148 | |
| 149 | |
| 150 | |
| 151 | |
| 152 | |
| 153 | |
| 154 | |
| 155 | |
| 156 | |
| 157 | |
| 158 | |
| 159 | |
| 160 | |
| 161 | |
| 162 | |
| 163 | |
| 164 | |
| 165 | |
| 166 | |
| 167 | |
| 168 | |
| 169 | |
| 170 | |
| 171 | |
| 172 | |
| 173 | |
| 174 | |
| 175 | |
| 176 | |
| 177 | |
| 178 | |
| 179 | |
| 180 | |
| 181 | |
| 182 | |
| 183 | |
| 184 | |
| 185 | |
| 186 | |
| 187 | |
| 188 | |
| 189 | |
| 190 | |
| 191 | |
| 192 | |
| 193 | |
| 194 | |
| 195 | |
| 196 | |
| 197 | |
| 198 | |
| 199 | |
| 200 | |
| 201 | |
| 202 | |
| 203 | |
| 204 | |
| 205 | |
| 206 | |
| 207 | |
| 208 | |
| 209 | |
| 210 | |
| 211 | |
| 212 | |
| 213 | |
| 214 | |
| 215 | |
| 216 | |
| 217 | |
| 218 | |
| 219 | |
| 220 | |
| 221 | |
| 222 | |
| 223 | |
| 224 | |
| 225 | |
| 226 | |
| 227 | |
| 228 | |
| 229 | |
| 230 | |
| 231 | |
| 232 | |
| 233 | |
| 234 | |
| 235 | |
| 236 | |
| 237 | |
| 238 | |
| 239 | |
| 240 | |
| 241 | |
| 242 | |
| 243 | |
| 244 | |
| 245 | |
| 246 | |
| 247 | |
| 248 | |
| 249 | |
| 250 | |
| 251 | |
| 252 | |
| 253 | |
| 254 | |
| 255 | |
| 256 | |
| 257 | |
| 258 | |
| 259 | AGI_HUM1_OLIGO_A_23_P32 [symbol] |
| 260 | |
| 261 | |
| 262 | |
| 263 | |
| 264 | |
| 265 | |
| 266 | |
| 267 | |
| 268 | |
| 269 | |
| 270 | |
| 271 | |
| 272 | |
| 273 | |
| 274 | |
| 275 | |
| 276 | |
| 277 | |
| 278 | |
| 279 | |
| 280 | |
| 281 | |
| 282 | |
| 283 | |
| 284 | |
| 285 | |
| 286 | |
| 287 | |
| 288 | |
| 289 | |
| 290 | |
| 291 | |
| 292 | |
| 293 | |
| 294 | |
| 295 | |
| 296 | |
| 297 | |
| 298 | |
| 299 | |
| 300 | |
| 301 | |
| 302 | |
| 303 | |
| 304 | |
| 305 | |
| 306 | |
| 307 | |
| 308 | |
| 309 | |
| 310 | |
| 311 | |
| 312 | |
| 313 | |
| 314 | |
| 315 | |
| 316 | |
| 317 | |
| 318 | |
| 319 | |
| 320 | |
| 321 | |
| 322 | |
| 323 | |
| 324 | |
| 325 | |
| 326 | |
| 327 | |
| 328 | |
| 329 | |
| 330 | |
| 331 | |
| 332 | |
| 333 | |
| 334 | |
| 335 | |
| 336 | |
| 337 | |
| 338 | |
| 339 | |
| 340 | |
| 341 | |
| 342 | |
| 343 | |
| 344 | |
| 345 | |
| 346 | |
| 347 | |
| 348 | |
| 349 | |
| 350 | |
| 351 | |
| 352 | |
| 353 | |
| 354 | |
| 355 | |
| 356 | |
| 357 | |
| 358 | |
| 359 | |
| 360 | |
| 361 | |
| 362 | |
| 363 | |
| 364 | |
| 365 | |
| 366 | |
| 367 | |
| 368 | |
| 369 | |
| 370 | |
| 371 | |
| 372 | |
| 373 | |
| 374 | |
| 375 | |
| 376 | |
| 377 | |
| 378 | |
| 379 | |
| 380 | |
| 381 | |
| 382 | |
| 383 | |
| 384 | |
| 385 | |
| 386 | |
| 387 | |
| 388 | |
| 389 | |
| 390 | |
| 391 | |
| 392 | |
| 393 | |
| 394 | |
| 395 | |
| 396 | |
| 397 | |
| 398 | |
| 399 | |
| 400 | |
| 401 | |
| 402 | |
| 403 | |
| 404 | |
| 405 | |
| 406 | |
| 407 | |
| 408 | |
| 409 | |
| 410 | |
| 411 | |
| 412 | |
| 413 | |
| 414 | |
| 415 | |
| 416 | |
| 417 | |
| 418 | |
| 419 | |
| 420 | |
| 421 | |
| 422 | |
| 423 | |
| 424 | |
| 425 | |
| 426 | |
| 427 | |
| 428 | |
| 429 | |
| 430 | |
| 431 | |
| 432 | |
| 433 | |
| 434 | |
| 435 | |
| 436 | |
| 437 | |
| 438 | |
| 439 | |
| 440 | |
| 441 | |
| 442 | |
| 443 | |
| 444 | |
| 445 | |
| 446 | |
| 447 | |
| 448 | |
| 449 | |
| 450 | |
| 451 | |
| 452 | |
| 453 | |
| 454 | |
| 455 | |
| 456 | |
| 457 | |
| 458 | |
| 459 | |
| 460 | |
| 461 | |
| 462 | |
| 463 | |
| 464 | |
| 465 | |
| 466 | |
| 467 | |
| 468 | |
| 469 | |
| 470 | |
| 471 | |
| 472 | |
| 473 | |
| 474 | |
| 475 | |
| 476 | |
| 477 | |
| 478 | |
| 479 | |
| 480 | |
| 481 | |
| 482 | |
| 483 | |
| 484 | |
| 485 | |
| 486 | |
| 487 | |
| 488 | |
| 489 | |
| 490 | |
| 491 | |
| 492 | |
| 493 | |
| 494 | |
| 495 | |
| 496 | |
| 497 | |
| 498 | |
| 499 | |
| 500 | |
| 501 | |
| 502 | |
| 503 | |
| 504 | |
| 505 | |
| 506 | |
| 507 | |
| 508 | |
| 509 | |
| 510 | |
| 511 | |
| 512 | |
| 513 | |
| 514 | |
| 515 | |
| 516 | |
| 517 | |
| 518 | |
| 519 | |
| 520 | |
| 521 | |
| 522 | |
| 523 | |
| 524 | |
| 525 | |
| 526 | |
| 527 | |
| 528 | |
| 529 | |
| 530 | |
| 531 | |
| 532 | |
| 533 | |
| 534 | |
| 535 | |
| 536 | |
| 537 | |
| 538 | |
| 539 | |
| 540 | |
| 541 | |
| 542 | |
| 543 | |
| 544 | |
| 545 | |
| 546 | |
| 547 | |
| 548 | |
| 549 | |
| 550 | |
| 551 | |
| 552 | |
| 553 | |
| 554 | |
| 555 | |
| 556 | |
| 557 | |
| 558 | |
| 559 | |
| 560 | |
| 561 | |
| 562 | |
| 563 | |
| 564 | |
| 565 | |
| 566 | |
| 567 | |
| 568 | |
| 569 | |
| 570 | |
| 571 | |
| 572 | |
| 573 | |
| 574 | |
| 575 | |
| 576 | |
| 577 | |
| 578 | |
| 579 | |
| 580 | |
| 581 | |
| 582 | |
| 583 | |
| 584 | |
| 585 | |
| 586 | |
| 587 | |
| 588 | |
| 589 | |
| 590 | |
| 591 | |
| 592 | |
| 593 | |
| 594 | |
| 595 | |
| 596 | |
| 597 | |
| 598 | |
| 599 | |
| 600 | |
| 601 | |
| 602 | |
| 603 | |
| 604 | |
| 605 | |
| 606 | |
| 607 | |
| 608 | |
| 609 | |
| 610 | |
| 611 | |
| 612 | |
| 613 | |
| 614 | |
| 615 | |
| 616 | |
| 617 | |
| 618 | |
| 619 | |
| 620 | |
| 621 | |
| 622 | |
| 623 | |
| 624 | |
| 625 | |
| 626 | |
| 627 | |
| 628 | |
| 629 | |
| 630 | |
| 631 | |
| 632 | |
| 633 | |
| 634 | |
| 635 | |
| 636 | |
| 637 | |
| 638 | |
| 639 | |
| 640 | |
| 641 | |
| 642 | |
| 643 | |
| 644 | |
| 645 | |
| 646 | |
| 647 | |
| 648 | |
| 649 | |
| 650 | |
| 651 | |
| 652 | |
| 653 | |
| 654 | |
| 655 | |
| 656 | |
| 657 | |
| 658 | |
| 659 | |
| 660 | |
| 661 | |
| 662 | |
| 663 | |
| 664 | |
| 665 | |
| 666 | |
| 667 | |
| 668 | |
| 669 | |
| 670 | |
| 671 | |
| 672 | |
| 673 | |
| 674 | |
| 675 | |
| 676 | |
| 677 | |
| 678 | |
| 679 | |
| 680 | |
| 681 | |
| 682 | |
| 683 | |
| 684 | |
| 685 | |
| 686 | |
| 687 | |
| 688 | |
| 689 | |
| 690 | |
| 691 | |
| 692 | |
| 693 | |
| 694 | |
| 695 | |
| 696 | |
| 697 | |
| 698 | |
| 699 | |
| 700 | |
| 701 | |
| 702 | |
| 703 | |
| 704 | |
| 705 | |
| 706 | |
| 707 | |
| 708 | |
| 709 | |
| 710 | |
| 711 | |
| 712 | |
| 713 | |
| 714 | |
| 715 | |
| 716 | |
| 717 | |
| 718 | |
| 719 | |
| 720 | |
| 721 | |
| 722 | |
| 723 | |
| 724 | |
| 725 | |
| 726 | |
| 727 | |
| 728 | |
| 729 | |
| 730 | |
| 731 | |
| 732 | |
| 733 | |
| 734 | |
| 735 | |
| 736 | |
| 737 | |
| 738 | |
| 739 | |
| 740 | |
| 741 | |
| 742 | |
| 743 | |
| 744 | |
| 745 | |
| 746 | |
| 747 | |
| 748 | |
| 749 | |
| 750 | |
| 751 | |
| 752 | |
| 753 | |
| 754 | |
| 755 | |
| 756 | |
| 757 | |
| 758 | |
| 759 | |
| 760 | |
| 761 | |
| 762 | |
| 763 | |
| 764 | |
| 765 | |
| 766 | |
| 767 | |
| 768 | |
| 769 | |
| 770 | |
| 771 | |
| 772 | |
| 773 | |
| 774 | |
| 775 | |
| 776 | |
| 777 | |
| 778 | |
| 779 | |
| 780 | |
| 781 | |
| 782 | |
| 783 | |
| 784 | |
| 785 | |
| 786 | |
| 787 | |
| 788 | |
| 789 | |
| 790 | |
| 791 | |
| 792 | |
| 793 | |
| 794 | |
| 795 | |
| 796 | |
| 797 | |
| 798 | |
| 799 | |
| 800 | |
| 801 | |
| 802 | |
| 803 | |
| 804 | |
| 805 | |
| 806 | |
| 807 | |
| 808 | |
| 809 | |
| 810 | |
| 811 | |
| 812 | |
| 813 | |
| 814 | |
| 815 | |
| 816 | |
| 817 | |
| 818 | |
| 819 | |
| 820 | |
| 821 | |
| 822 | |
| 823 | |
| 824 | |
| 825 | |
| 826 | |
| 827 | |
| 828 | |
| 829 | |
| 830 | |
| 831 | |
| 832 | |
| 833 | |
| 834 | |
| 835 | |
| 836 | |
| 837 | |
| 838 | |
| 839 | |
| 840 | |
| 841 | |
| 842 | |
| 843 | |
| 844 | |
| 845 | |
| 846 | |
| 847 | |
| 848 | |
| 849 | |
| 850 | |
| 851 | |
| 852 | |
| 853 | |
| 854 | |
| 855 | |
| 856 | |
| 857 | |
| 858 | |
| 859 | |
| 860 | |
| 861 | |
| 862 | |
| 863 | |
| 864 | |
| 865 | |
| 866 | |
| 867 | |
| 868 | |
| 869 | |
| 870 | |
| 871 | |
| 872 | |
| 873 | |
| 874 | |
| 875 | |
| 876 | |
| 877 | |
| 878 | |
| 879 | |
| 880 | |
| 881 | |
| 882 | |
| 883 | |
| 884 | |
| 885 | |
| 886 | |
| 887 | |
| 888 | |
| 889 | |
| 890 | |
| 891 | |
| 892 | |
| 893 | |
| 894 | |
| 895 | |
| 896 | |
| 897 | |
| 898 | |
| 899 | |
| 900 | |
| 901 | |
| 902 | |
| 903 | |
| 904 | |
| 905 | |
| 906 | |
| 907 | |
| 908 | |
| 909 | |
| 910 | |
| 911 | |
| 912 | |
| 913 | |
| 914 | |
| 915 | |
| 916 | |
| 917 | |
| 918 | |
| 919 | |
| 920 | |
| 921 | |
| 922 | |
| 923 | |
| 924 | |
| 925 | |
| 926 | |
| 927 | |
| 928 | |
| 929 | |
| 930 | |
| 931 | |
| 932 | |
| 933 | |
| 934 | |
| 935 | |
| 936 | |
| 937 | |
| 938 | |
| 939 | |
| 940 | |
| 941 | |
| 942 | |
| 943 | |
| 944 | |
| 945 | |
| 946 | |
| 947 | |
| 948 | |
| 949 | |
| 950 | |
| 951 | |
| 952 | |
| 953 | |
| 954 | |
| 955 | |
| 956 | |
| 957 | |
| 958 | |
| 959 | |
| 960 | |
| 961 | |
| 962 | |
| 963 | |
| 964 | |
| 965 | |
| 966 | |
| 967 | |
| 968 | |
| 969 | |
| 970 | |
| 971 | |
| 972 | |
| 973 | |
| 974 | |
| 975 | |
| 976 | |
| 977 | |
| 978 | |
| 979 | |
| 980 | |
| 981 | |
| 982 | |
| 983 | |
| 984 | |
| 985 | |
| 986 | |
| 987 | |
| 988 | |
| 989 | |
| 990 | |
| 991 | |
| 992 | |
| 993 | |
| 994 | |
| 995 | |
| 996 | |
| 997 | |
| 998 | |
| 999 | |
| 1000 | |
| 1001 | |
| 1002 | |
| 1003 | |
| 1004 | |
| 1005 | |
| 1006 | |
| 1007 | |
| 1008 | |
| 1009 | |
| 1010 | |
| 1011 | |
| 1012 | |
| 1013 | |
| 1014 | |
| 1015 | |
| 1016 | |
| 1017 | |
| 1018 | |
| 1019 | |
| 1020 | |
| 1021 | |
| 1022 | |
| 1023 | |
| 1024 | |
| 1025 | |
| 1026 | |
| 1027 | |
| 1028 | |
| 1029 | |
| 1030 | |
| 1031 | |
| 1032 | |
| 1033 | |
| 1034 | |
| 1035 | |
| 1036 | |
| 1037 | |
| 1038 | |
| 1039 | |
| 1040 | |
| 1041 | |
| 1042 | |
| 1043 | |
| 1044 | |
| 1045 | |
| 1046 | |
| 1047 | |
| 1048 | |
| 1049 | |
| 1050 | |
| 1051 | |
| 1052 | |
| 1053 | |
| 1054 | |
| 1055 | |
| 1056 | |
| 1057 | |
| 1058 | |
| 1059 | |
| 1060 | |
| 1061 | |
| 1062 | |
| 1063 | |
| 1064 | |
| 1065 | |
| 1066 | |
| 1067 | |
| 1068 | |
| 1069 | |
| 1070 | |
| 1071 | |
| 1072 | |
| 1073 | |
| 1074 | |
| 1075 | |
| 1076 | |
| 1077 | |
| 1078 | |
| 1079 | |
| 1080 | |
| 1081 | |
| 1082 | |
| 1083 | |
| 1084 | |
| 1085 | |
| 1086 | |
| 1087 | |
| 1088 | |
| 1089 | |
| 1090 | |
| 1091 | |
| 1092 | |
| 1093 | |
| 1094 | |
| 1095 | |
| 1096 | |
| 1097 | |
| 1098 | |
| 1099 | |
| 1100 | |
| 1101 | |
| 1102 | |

[symbol]

indicates data missing or illegible when filed

TABLE 14

| | Diagonal | Diagonal | Diagonal | Concordance | Diagonal | Lowe | Upper | |
|---|---|---|---|---|---|---|---|---|
| | bias | corr. | spread | corr. | std. dev | Limits | Limits | Range |
| Gene | Raw | Raw | Raw | Raw | Raw | Raw | Raw | Raw |
| ACTB | −1.6 | −0.012 | 0.85 | −0.004 | 1.1 | 0.026 | 1.8 | 6.5 |
| ASF1A | −2.3 | 0.044 | 1.1 | 0.012 | 1.4 | 0.0067 | 1.5 | 5.9 |
| B3GNT5 | −0.51 | 0.49 | 0.89 | 0.45 | 1.1 | 0.064 | 5.7 | 8.6 |
| BLVRA | −2.1 | 0.055 | 1.1 | 0.021 | 1.6 | 0.0054 | 3 | 7.8 |
| BTG3 | −1.1 | 0.39 | 0.84 | 0.25 | 1.1 | 0.038 | 3.3 | 7.6 |
| BUB1 | −1.5 | 0.51 | 0.68 | 0.23 | 0.94 | 0.038 | 1.5 | 5.3 |
| C10ORF7 | −2.1 | 0.18 | 0.86 | 0.045 | 1.1 | 0.015 | 1.1 | 5.7 |
| C16ORF45 | −2.8 | 0.31 | 1.1 | 0.093 | 1.5 | 0.0032 | 1.3 | 9.2 |
| CaMKIIN/Alpha | −0.91 | 0.69 | 0.68 | 0.51 | 0.87 | 0.075 | 2.2 | 8.3 |
| CDH3 | −1.5 | 0.65 | 0.96 | 0.45 | 1.3 | 0.02 | 2.9 | 9.7 |
| CHI3L2 | −1.5 | 0.84 | 0.91 | 0.66 | 1.1 | 0.024 | 2 | 13 |
| COX6C | −2.2 | 0.59 | 1 | 0.28 | 1.3 | 0.0081 | 1.6 | 10 |
| CSDA | −1.1 | 0.35 | 0.92 | 0.24 | 1.4 | 0.022 | 4.7 | 6.1 |
| CTPS | −1.8 | 0.27 | 0.83 | 0.096 | 1.1 | 0.018 | 1.6 | 5.4 |
| ERBB2 | −0.65 | 0.73 | 0.72 | 0.64 | 0.91 | 0.088 | 3.2 | 8.1 |
| ESR1 | −1.6 | 0.9 | 0.8 | 0.74 | 1 | 0.028 | 1.6 | 13 |
| FABP7 | −0.9 | 0.81 | 1.4 | 0.78 | 2 | 0.0086 | 20 | 23 |
| FBP1 | −1.8 | 0.67 | 0.9 | 0.37 | 1.2 | 0.016 | 1.6 | 10 |
| FLJ10980 | −2.8 | 0.42 | 0.83 | 0.086 | 1.1 | 0.0074 | 0.54 | 6.1 |
| FOXC1 | −0.034 | 0.72 | 0.83 | 0.72 | 1.1 | 0.11 | 8.9 | 8.2 |
| FZD7 | −0.93 | 0.3 | 0.7 | 0.17 | 0.92 | 0.067 | 2.4 | 5.8 |
| GATA3 | −1.5 | 0.77 | 0.88 | 0.53 | 1.1 | 0.026 | 1.9 | 11 |
| GRB7 | −1.4 | 0.73 | 0.7 | 0.46 | 0.94 | 0.042 | 1.7 | 8.5 |
| GSTM3 | −2.3 | 0.65 | 0.86 | 0.25 | 1.1 | 0.012 | 0.83 | 9 |
| GSTP1 | −0.033 | 0.51 | 0.76 | 0.51 | 0.97 | 0.14 | 6.5 | 5.1 |
| HIS1 | −1.3 | 0.3 | 0.75 | 0.13 | 0.94 | 0.046 | 1.8 | 6 |
| ID4 | −1.1 | 0.64 | 0.74 | 0.44 | 0.95 | 0.055 | 2.3 | 6.9 |
| IGBP1 | −2.5 | −0.16 | 1.1 | −0.035 | 1.5 | 0.0048 | 1.4 | 6.1 |
| INPP4B | −1.5 | 0.67 | 0.81 | 0.4 | 1.1 | 0.027 | 1.8 | 10 |
| KIT | −1.7 | 0.28 | 1.2 | 0.16 | 1.7 | 0.0071 | 5.5 | 9.4 |
| KRT17 | −0.7 | 0.66 | 0.92 | 0.59 | 1.2 | 0.049 | 5.1 | 9 |
| MKI67 | −1.3 | 0.48 | 0.7 | 0.24 | 0.92 | 0.047 | 1.7 | 5.7 |
| MRPL19 | −0.61 | 0.0035 | 0.88 | 0.0027 | 1.1 | 0.062 | 4.9 | 4.7 |
| MYBL2 | −0.18 | 0.8 | 0.57 | 0.79 | 0.76 | 0.19 | 3.7 | 5.6 |
| PSMC4_R3 | −2.6 | −0.061 | 1 | −0.012 | 1.3 | 0.0056 | 1 | 6.1 |
| PUM1 | −2.5 | −0.15 | 0.92 | −0.026 | 1.2 | 0.0084 | 0.97 | 6.2 |
| S100A11 | −0.89 | 0.34 | 0.86 | 0.23 | 1 | 0.054 | 3.3 | 5.8 |
| SEMA3C | −1.1 | 0.46 | 0.77 | 0.29 | 1.1 | 0.042 | 2.7 | 7.3 |
| SF3A1 | −2.3 | −0.24 | 0.93 | −0.043 | 1.2 | 0.0095 | 1.1 | 6 |
| SLC39A6 | −1.5 | 0.76 | 0.78 | 0.49 | 0.99 | 0.033 | 1.6 | 7.8 |
| SLC5A6 | −1.3 | 0.59 | 0.61 | 0.27 | 0.79 | 0.058 | 1.3 | 5.1 |
| STK6 | −1.4 | 0.63 | 0.63 | 0.32 | 0.87 | 0.046 | 1.4 | 5.7 |
| TCEAL1 | −1.2 | 0.47 | 0.64 | 0.24 | 0.85 | 0.061 | 1.7 | 6.1 |
| TFF3 | −1.8 | 0.8 | 0.89 | 0.55 | 1.2 | 0.016 | 1.8 | 10 |
| TMSB10 | −2.3 | 0.21 | 0.97 | 0.063 | 1.3 | 0.0082 | 1.4 | 6 |
| TOP2A | −2 | 0.53 | 0.9 | 0.23 | 1.2 | 0.014 | 1.5 | 7.3 |
| TP53BP2 | −0.93 | 0.47 | 0.74 | 0.3 | 0.89 | 0.07 | 2.3 | 5.3 |
| VAV3 | −3.1 | 0.48 | 0.97 | 0.12 | 1.3 | 0.0037 | 0.6 | 8.5 |
| WWP1 | −1.7 | 0.55 | 0.74 | 0.21 | 0.94 | 0.029 | 1.1 | 7.1 |
| XBP1 | −0.87 | 0.67 | 0.81 | 0.54 | 1 | 0.057 | 3.2 | N/A |

| | Diagonal | Diagonal | Diagonal | Concordance | Diagonal | Lowe | Upper | | Diagonal |
|---|---|---|---|---|---|---|---|---|---|
| | bias | corr. | spread | corr. | std. dev | Limits | Limits | Range | bias |
| Gene | Norm | Norm | Norm | Norm | Norm | Norm | Norm | Norm | DWD |
| ACTB | N/A | N/A | N/A | N/A | N/A | N/A | N/A | N/A | N/A |
| ASF1A | −0.41 | 0.43 | 0.38 | 0.31 | 0.51 | 0.24 | 1.8 | 2.1 | 0.061 |
| B3GNT5 | 1.5 | 0.63 | 0.64 | 0.26 | 0.76 | 0.97 | 19 | 4.8 | 0.24 |
| BLVRA | −0.16 | 0.46 | 0.52 | 0.45 | 0.83 | 0.17 | 4.4 | 4.6 | −0.3 |
| BTG3 | 0.91 | 0.39 | 0.64 | 0.24 | 0.9 | 0.42 | 14 | 6.6 | 0.47 |
| BUB1 | 0.43 | 0.59 | 0.56 | 0.51 | 0.7 | 0.39 | 6 | 4.4 | 0.42 |
| C10ORF7 | −0.14 | 0.7 | 0.28 | 0.67 | 0.38 | 0.42 | 1.8 | 3.6 | 0.36 |
| C16ORF45 | −0.74 | 0.74 | 0.59 | 0.61 | 0.82 | 0.097 | 2.4 | 6.4 | 0.079 |
| CaMKIIN/Alpha | 1.1 | 0.67 | 0.6 | 0.44 | 0.91 | 0.51 | 18 | 6.5 | 0.001 |
| CDH3 | 0.48 | 0.82 | 0.56 | 0.76 | 0.72 | 0.39 | 6.6 | 8.4 | 0.64 |
| CHI3L2 | 0.51 | 0.8 | 0.88 | 0.78 | 1.2 | 0.16 | 17 | 12 | 0.95 |
| COX6C | −0.28 | 0.93 | 0.39 | 0.91 | 0.48 | 0.3 | 1.9 | 6.4 | −0.23 |
| CSDA | 0.79 | 0.51 | 0.69 | 0.39 | 0.96 | 0.33 | 14 | 7.6 | 0.12 |
| CTPS | 0.12 | 0.56 | 0.47 | 0.54 | 0.59 | 0.35 | 3.6 | 3.3 | 0.3 |
| ERBB2 | 1.3 | 0.75 | 0.71 | 0.48 | 0.9 | 0.64 | 22 | 7 | 0.24 |
| ESR1 | 0.52 | 0.88 | 0.69 | 0.86 | 1.2 | 0.17 | 16 | 9.2 | −0.17 |
| FABP7 | 1.1 | 0.74 | 1.3 | 0.67 | 1.8 | 0.093 | 97 | 14 | 1.3 |
| FBP1 | 0.18 | 0.89 | 0.41 | 0.88 | 0.62 | 0.36 | 4 | 7.3 | −0.13 |
| FLJ10980 | −0.82 | 0.73 | 0.51 | 0.52 | 0.65 | 0.12 | 1.6 | 4.7 | 0.26 |
| FOXC1 | 1.8 | 0.77 | 0.58 | 0.36 | 0.81 | 1.2 | 29 | 6.2 | −0.055 |
| FZD7 | 1.1 | 0.47 | 0.58 | 0.21 | 0.72 | 0.72 | 12 | 4.2 | 0.13 |
| GATA3 | 0.58 | 0.72 | 0.84 | 0.68 | 1.3 | 0.14 | 22 | 10 | 0.25 |
| GRB7 | 0.65 | 0.78 | 0.66 | 0.69 | 0.85 | 0.35 | 10 | 6.8 | 0.49 |
| GSTM3 | −0.26 | 0.74 | 0.6 | 0.73 | 0.97 | 0.12 | 5.2 | 7.4 | 0.27 |
| GSTP1 | 2 | 0.53 | 0.64 | 0.13 | 0.77 | 1.5 | 31 | 4.2 | 0.014 |
| HIS1 | 0.71 | 0.42 | 0.56 | 0.26 | 0.7 | 0.5 | 7.9 | 3.3 | 0.1 |
| ID4 | 0.93 | 0.56 | 0.8 | 0.41 | 1 | 0.33 | 19 | 7.5 | 0.85 |
| IGBP1 | −0.59 | 0.37 | 0.44 | 0.23 | 0.61 | 0.17 | 1.8 | 3.8 | −0.035 |
| INPP4B | 0.51 | 0.86 | 0.47 | 0.79 | 0.64 | 0.47 | 5.8 | 5.8 | 0.019 |
| KIT | 0.71 | 0.66 | 0.61 | 0.54 | 0.86 | 0.37 | 11 | 6 | 0.65 |
| KRT17 | 1.4 | 0.57 | 0.91 | 0.38 | 1.3 | 0.32 | 47 | 9.2 | 0.91 |
| MKI67 | 0.66 | 0.41 | 0.63 | 0.29 | 0.78 | 0.41 | 8.8 | 3.4 | 0.5 |
| MRPL19 | N/A | N/A | N/A | N/A | N/A | N/A | N/A | N/A | N/A |
| MYBL2 | 1.7 | 0.65 | 0.73 | 0.3 | 0.95 | 0.83 | 34 | 6.2 | 0.15 |
| PSMC4_R3 | N/A | N/A | N/A | N/A | N/A | N/A | N/A | N/A | N/A |
| PUM1 | N/A | N/A | N/A | N/A | N/A | N/A | N/A | N/A | N/A |
| S100A11 | 1.1 | 0.48 | 0.57 | 0.2 | 0.7 | 0.78 | 12 | 3.3 | 0.092 |
| SEMA3C | 0.97 | 0.62 | 0.67 | 0.43 | 0.9 | 0.44 | 15 | 6 | 0.2 |
| SF3A1 | N/A | N/A | N/A | N/A | N/A | N/A | N/A | N/A | N/A |
| SLC39A6 | 0.51 | 0.85 | 0.62 | 0.8 | 0.81 | 0.34 | 8.1 | 6 | 0.2 |
| SLC5A6 | 0.65 | 0.56 | 0.62 | 0.42 | 0.77 | 0.42 | 8.7 | 5.6 | 0.58 |
| STK6 | 0.53 | 0.65 | 0.57 | 0.55 | 0.74 | 0.39 | 7.2 | 5.2 | 0.48 |
| TCEAL1 | 0.82 | 0.56 | 0.61 | 0.39 | 0.82 | 0.44 | 11 | 4.9 | 0.36 |
| TFF3 | 0.18 | 0.89 | 0.7 | 0.89 | 0.88 | 0.21 | 6.7 | 9.4 | −0.025 |
| TMSB10 | −0.34 | 0.54 | 0.41 | 0.46 | 0.53 | 0.25 | 2 | 2.5 | 0.18 |
| TOP2A | −0.5 | 0.55 | 0.94 | 0.52 | 1.3 | 0.048 | 7.7 | 6.6 | 0.41 |
| TP53BP2 | 1.1 | 0.54 | 0.55 | 0.26 | 0.68 | 0.75 | 11 | 4.6 | 0.3 |
| VAV3 | −1.1 | 0.65 | 0.62 | 0.47 | 1 | 0.047 | 2.6 | 7.8 | 0.35 |
| WWP1 | 0.26 | 0.73 | 0.55 | 0.7 | 0.67 | 0.34 | 4.9 | 5.4 | 0.22 |
| XBP1 | 1.2 | 0.7 | 0.71 | 0.48 | 1 | 0.46 | 23 | 6.6 | −0.1 |

| | Diagonal | Diagonal | Concordance | Diagonal | Lowe | Upper | |
|---|---|---|---|---|---|---|---|
| | corr. | spread | corr. | std. dev | Limits | Limits | Range |
| Gene | DWD | DWD | DWD | DWD | DWD | DWD | DWD |
| ACTB | N/A | N/A | N/A | N/A | N/A | N/A | N/A |
| ASF1A | 0.43 | 0.38 | 0.42 | 0.51 | 0.39 | 2.9 | 2.1 |
| B3GNT5 | 0.63 | 0.64 | 0.61 | 0.76 | 0.28 | 5.6 | 4.8 |
| BLVRA | 0.46 | 0.52 | 0.43 | 0.83 | 0.15 | 3.8 | 4.6 |
| BTG3 | 0.39 | 0.64 | 0.34 | 0.9 | 0.27 | 9.3 | 6.6 |
| BUB1 | 0.59 | 0.56 | 0.51 | 0.7 | 0.38 | 5.9 | 4.4 |
| C10ORF7 | 0.7 | 0.28 | 0.55 | 0.38 | 0.68 | 3 | 3.6 |
| C16ORF45 | 0.74 | 0.59 | 0.74 | 0.82 | 0.22 | 5.4 | 6.4 |
| CaMKIIN/Alpha | 0.67 | 0.6 | 0.67 | 0.91 | 0.17 | 5.9 | 6.5 |
| CDH3 | 0.82 | 0.56 | 0.73 | 0.72 | 0.45 | 7.7 | 8.4 |
| CHI3L2 | 0.8 | 0.88 | 0.71 | 1.2 | 0.25 | 26 | 12 |
| COX6C | 0.93 | 0.39 | 0.91 | 0.48 | 0.31 | 2 | 6.4 |
| CSDA | 0.51 | 0.69 | 0.51 | 0.96 | 0.17 | 7.4 | 7.6 |
| CTPS | 0.56 | 0.47 | 0.5 | 0.59 | 0.42 | 4.3 | 3.3 |
| ERBB2 | 0.75 | 0.71 | 0.73 | 0.9 | 0.22 | 7.4 | 7 |
| ESR1 | 0.88 | 0.69 | 0.88 | 1.2 | 0.088 | 8.1 | 9.2 |
| FABP7 | 0.74 | 1.3 | 0.65 | 1.8 | 0.11 | 120 | 14 |
| FBP1 | 0.89 | 0.41 | 0.88 | 0.62 | 0.26 | 2.9 | 7.3 |
| FLJ10980 | 0.73 | 0.51 | 0.7 | 0.65 | 0.36 | 4.6 | 4.7 |
| FOXC1 | 0.77 | 0.58 | 0.77 | 0.81 | 0.2 | 4.6 | 6.2 |
| FZD7 | 0.47 | 0.58 | 0.47 | 0.72 | 0.28 | 4.7 | 4.2 |
| GATA3 | 0.72 | 0.84 | 0.71 | 1.3 | 0.1 | 16 | 10 |
| GRB7 | 0.78 | 0.66 | 0.72 | 0.85 | 0.3 | 8.6 | 6.8 |
| GSTM3 | 0.74 | 0.6 | 0.73 | 0.97 | 0.2 | 8.6 | 7.4 |
| GSTP1 | 0.53 | 0.64 | 0.53 | 0.77 | 0.23 | 4.5 | 4.2 |
| HIS1 | 0.42 | 0.56 | 0.41 | 0.7 | 0.28 | 4.4 | 3.3 |
| ID4 | 0.56 | 0.8 | 0.43 | 1 | 0.3 | 17 | 7.5 |
| IGBP1 | 0.37 | 0.44 | 0.37 | 0.61 | 0.29 | 3.2 | 3.8 |
| INPP4B | 0.86 | 0.47 | 0.86 | 0.64 | 0.29 | 3.6 | 5.8 |
| KIT | 0.66 | 0.61 | 0.56 | 0.86 | 0.35 | 10 | 6 |
| KRT17 | 0.57 | 0.91 | 0.47 | 1.3 | 0.2 | 29 | 9.2 |
| MKI67 | 0.41 | 0.63 | 0.33 | 0.78 | 0.35 | 7.5 | 3.4 |
| MRPL19 | N/A | N/A | N/A | N/A | N/A | N/A | N/A |
| MYBL2 | 0.65 | 0.73 | 0.64 | 0.95 | 0.18 | 7.4 | 6.2 |
| PSMC4_R3 | N/A | N/A | N/A | N/A | N/A | N/A | N/A |
| PUM1 | N/A | N/A | N/A | N/A | N/A | N/A | N/A |
| S100A11 | 0.48 | 0.57 | 0.47 | 0.7 | 0.28 | 4.3 | 3.3 |
| SEMA3C | 0.62 | 0.67 | 0.61 | 0.9 | 0.21 | 7.1 | 6 |
| SF3A1 | N/A | N/A | N/A | N/A | N/A | N/A | N/A |
| SLC39A6 | 0.85 | 0.62 | 0.84 | 0.81 | 0.25 | 6 | 6 |
| SLC5A6 | 0.56 | 0.62 | 0.44 | 0.77 | 0.39 | 8.1 | 5.6 |
| STK6 | 0.65 | 0.57 | 0.56 | 0.74 | 0.37 | 6.9 | 5.2 |
| TCEAL1 | 0.56 | 0.61 | 0.52 | 0.82 | 0.28 | 7.1 | 4.9 |
| TFF3 | 0.89 | 0.7 | 0.89 | 0.88 | 0.17 | 5.5 | 9.4 |
| TMSB10 | 0.54 | 0.41 | 0.51 | 0.53 | 0.42 | 3.4 | 2.5 |
| TOP2A | 0.55 | 0.94 | 0.53 | 1.3 | 0.12 | 19 | 6.6 |
| TP53BP2 | 0.54 | 0.55 | 0.5 | 0.68 | 0.35 | 5.1 | 4.6 |
| VAV3 | 0.65 | 0.62 | 0.62 | 1 | 0.19 | 10 | 7.8 |
| WWP1 | 0.73 | 0.55 | 0.71 | 0.67 | 0.33 | 4.6 | 5.4 |
| XBP1 | 0.7 | 0.71 | 0.7 | 1 | 0.13 | 6.5 | 6.6 |

TABLE 15

| | | | | | Prediction Analysis | |
|---|---|---|---|---|---|---|
| | Single Sample Predictor | | | | for Microarrys | |
| Sample | MA (1393) | MA (40) | PCR-FF (40) | PCR-FFPE (40) | MA (1393) | MA (40) |
| BR00-0284 | HER2+/ER− | HER2+/ER− | HER2+/ER− | HER2+/ER− | HER2+/ER− | HER2+/ER− |
| BR00-0365 | LUMINAL | LUMINAL | LUMINAL | LUMINAL | LUMINAL | LUMINAL |
| BR00-0572 | BASAL | BASAL | BASAL | BASAL | BASAL | BASAL |
| BR00-0587 | LUMINAL | LUMINAL | LUMINAL | LUMINAL | LUMINAL | LUMINAL |
| BR99-0207 | LUMINAL | LUMINAL | LUMINAL | LUMINAL | LUMINAL | LUMINAL |
| BR99-0348 | LUMINAL | LUMINAL | LUMINAL | LUMINAL | LUMINAL | LUMINAL |
| PB120-LN | BASAL | BASAL | BASAL | BASAL | BASAL | BASAL |
| PB126 | BASAL | BASAL | BASAL | BASAL | BASAL | BASAL |
| PB149 | LUMINAL | NORMAL-LIKE | NORMAL-LIKE | LUMINAL | LUMINAL | LUMINAL |
| PB205 | BASAL | BASAL | BASAL | BASAL | BASAL | BASAL |
| PB255 | LUMINAL | LUMINAL | LUMINAL | LUMINAL | LUMINAL | LUMINAL |
| PB297 | BASAL | BASAL | BASAL | BASAL | BASAL | BASAL |
| PB311 | HER2+/ER− | HER2+/ER− | HER2+/ER− | LUMINAL | LUMINAL | LUMINAL |
| PB314 | HER2+/ER− | HER2+/ER− | HER2+/ER− | HER2+/ER− | HER2+/ER− | HER2+/ER− |
| PB334 | BASAL | BASAL | BASAL | BASAL | BASAL | BASAL |
| PB362 | NORMAL-LIKE | NORMAL-LIKE | NORMAL-LIKE | LUMINAL | NORMAL-LIKE | NORMAL-LIKE |
| PB370 | LUMINAL | LUMINAL | LUMINAL | LUMINAL | LUMINAL | LUMINAL |
| PB376 | HER2+/ER− | BASAL | BASAL | BASAL | HER2+/ER− | BASAL |
| PB413 | LUMINAL | LUMINAL | LUMINAL | LUMINAL | LUMINAL | LUMINAL |
| PB441 | LUMINAL | LUMINAL | LUMINAL | LUMINAL | LUMINAL | LUMINAL |
| PB455 | HER2+/ER− | HER2+/ER− | HER2+/ER− | HER2+/ER− | HER2+/ER− | HER2+/ER− |
| UB29 | BASAL | BASAL | BASAL | BASAL | BASAL | BASAL |
| UB37 | HER2+/ER− | HER2+/ER− | HER2+/ER− | HER2+/ER− | HER2+/ER− | HER2+/ER− |
| UB38 | LUMINAL | LUMINAL | LUMINAL | LUMINAL | LUMINAL | LUMINAL |
| UB39 | LUMINAL | LUMINAL | LUMINAL | LUMINAL | LUMINAL | LUMINAL |
| UB43 | LUMINAL | LUMINAL | LUMINAL | LUMINAL | LUMINAL | LUMINAL |
| UB45 | LUMINAL | LUMINAL | LUMINAL | LUMINAL | LUMINAL | LUMINAL |
| UB55 | LUMINAL | LUMINAL | LUMINAL | LUMINAL | LUMINAL | LUMINAL |
| UB57 | LUMINAL | LUMINAL | LUMINAL | LUMINAL | LUMINAL | LUMINAL |
| UB58 | LUMINAL | LUMINAL | LUMINAL | LUMINAL | LUMINAL | LUMINAL |
| UB60 | LUMINAL | HER2+/ER− | HER2+/ER− | HER2+/ER− | HER2+/ER− | HER2+/ER− |
| UB66 | LUMINAL | LUMINAL | LUMINAL | LUMINAL | LUMINAL | LUMINAL |
| UB67 | BASAL | BASAL | BASAL | BASAL | BASAL | BASAL |
| UB71 | BASAL | BASAL | BASAL | BASAL | BASAL | BASAL |
| UB79 | LUMINAL | LUMINAL | LUMINAL | LUMINAL | LUMINAL | LUMINAL |

| | Prediction Analysis | | Immunohisto- | | | |
|---|---|---|---|---|---|---|
| | for Microarrys | | chemistry | | | HER2 DNA |
| Sample | PCR-FF (40) | PCR-FFPE (40) | ER | PR | HER2 | PCR |
| BR00-0284 | HER2+/ER− | HER2+/ER− | − | − | + | |
| BR00-0365 | LUMINAL | LUMINAL | + | + | + | |
| BR00-0572 | BASAL | BASAL | − | − | + | [symbol] |
| BR00-0587 | LUMINAL | LUMINAL | + | + | + | |
| BR99-0207 | LUMINAL | LUMINAL | + | − | − | |
| BR99-0348 | LUMINAL | LUMINAL | + | + | + | |
| PB120-LN | BASAL | BASAL | − | − | − | |
| PB126 | BASAL | BASAL | − | − | − | |
| PB149 | LUMINAL | LUMINAL | + | + | + | |
| PB205 | BASAL | BASAL | − | − | + | [symbol] |
| PB255 | LUMINAL | LUMINAL | + | + | + | |
| PB297 | BASAL | BASAL | − | − | − | |
| PB311 | LUMINAL | LUMINAL | + | + | − | |
| PB314 | HER2+/ER− | HER2+/ER− | − | − | + | ↑ |
| PB334 | BASAL | BASAL | − | − | − | |
| PB362 | NORMAL-LIKE | LUMINAL | + | + | − | |
| PB370 | LUMINAL | LUMINAL | + | + | − | |
| PB376 | BASAL | BASAL | − | − | + | [symbol] |
| PB413 | LUMINAL | LUMINAL | + | + | − | |
| PB441 | LUMINAL | LUMINAL | + | + | − | |
| PB455 | HER2+/ER− | HER2+/ER− | − | − | + | [symbol] |
| UB29 | BASAL | BASAL | − | − | − | |
| UB37 | HER2+/ER− | HER2+/ER− | − | + | + | |
| UB38 | LUMINAL | LUMINAL | + | + | − | ↑ |
| UB39 | LUMINAL | LUMINAL | + | − | − | |
| UB43 | LUMINAL | LUMINAL | + | + | − | ↑ |
| UB45 | LUMINAL | LUMINAL | + | + | − | |
| UB55 | LUMINAL | LUMINAL | + | + | + | |
| UB57 | LUMINAL | LUMINAL | + | + | − | |
| UB58 | LUMINAL | LUMINAL | + | + | − | |
| UB60 | HER2+/ER− | HER2+/ER− | − | − | + | ↑ |
| UB66 | LUMINAL | LUMINAL | + | + | − | |
| UB67 | BASAL | BASAL | − | − | − | |
| UB71 | BASAL | BASAL | − | − | − | |
| UB79 | LUMINAL | LUMINAL | + | − | − | |

TABLE 16
MA P3m test (40 g × 35 s) Gene Scores (threshold = 0)

| | | | |
|---|---|---|---|
| Offset Quantile | 50 | Offset Value | 0.650934012 |
| | both | RNG Seed | 420473 |

| | | Prior Distribution (Sample Prior) | | | |
|---|---|---|---|---|---|
| | | BASAL | HER2 | LUMINAL | NORMAL-LIKE |
| | Class | | | | |
| | Prob. | 0.225806 | 0.185483871 | 0.516129032 | 0.072580645 |
| | gene | | | | |
| 1 | ERBB2 | −0.3895 | 1.2282 | −0.1621 | −0.4533 |
| 2 | KRT17 | 0.4627 | −0.2412 | −0.2612 | 1.12 |
| 3 | KIT | 0.3877 | −0.2516 | −0.2197 | 1.0848 |
| 4 | ESR1 | −0.8232 | −0.9961 | 0.8007 | −0.3565 |
| 5 | FOXC1 | 0.9915 | −0.1912 | −0.4871 | 0.9538 |
| 6 | TFF3 | −0.9369 | 0 | 0.441 | −0.0307 |
| 7 | B3GNT5 | 0.9196 | 0.045 | −0.4806 | 0.2112 |
| 8 | XBP1 | −0.8375 | 0 | 0.44 | −0.4901 |
| 9 | GRB7 | 0 | 0.8365 | −0.2925 | 0.0187 |
| 10 | ID4 | 0.4412 | −0.1331 | −0.2462 | 0.8043 |
| 11 | COX6C | −0.2273 | −0.2661 | 0.3347 | −0.762 |
| 12 | TMSB10 | 0.2812 | 0.744 | −0.3593 | −0.2403 |
| 13 | GATA3 | −0.7118 | −0.2662 | 0.4969 | −0.4083 |
| 14 | SLC39A6 | −0.5871 | −0.7076 | 0.6434 | −0.7101 |
| 15 | WWP1 | −0.3005 | 0 | 0.2562 | −0.689 |
| 16 | FABP7 | 0.4915 | 0.1749 | −0.4044 | 0.6688 |
| 17 | CDH3 | 0.6578 | 0.0768 | −0.3978 | 0.3551 |
| 18 | BTG3 | 0.6466 | 0.0631 | −0.3522 | 0.1006 |
| 19 | CHI3L2 | 0.6145 | −0.072 | −0.2852 | 0.3859 |
| 20 | GSTP1 | 0.6044 | 0 | −0.2967 | 0.1985 |
| 21 | FBP1 | −0.6029 | −0.2298 | 0.4607 | −0.5828 |
| 22 | TP53BP2 | 0.5756 | 0.0568 | −0.2241 | −0.3619 |
| 23 | C10orf7 | 0.5399 | 0.1613 | −0.2739 | −0.1839 |
| 24 | SLC5A6 | 0.3315 | 0.5335 | −0.4014 | 0.2293 |
| 25 | FZD7 | 0.4426 | −0.1423 | −0.2053 | 0.5327 |
| 26 | ASF1A | 0.5161 | 0.1316 | −0.2183 | −0.409 |
| 27 | INPP4B | −0.5015 | 0 | 0.2227 | 0 |
| 28 | FLJ10980 | −0.4928 | 0.0031 | 0.2695 | −0.477 |
| 29 | CTPS | 0.4773 | 0.2509 | −0.3185 | 0 |
| 30 | GSTM3 | −0.4558 | 0.0512 | 0.1822 | −0.0945 |
| 31 | CSDA | 0.4181 | 0.1737 | −0.3408 | 0.4479 |
| 32 | SEMA3C | −0.4457 | −0.1442 | 0.2904 | −0.0793 |
| 33 | VAV3 | −0.4123 | −0.0092 | 0.278 | −0.4404 |
| 34 | S100A11 | 0.2956 | 0.3957 | −0.2321 | −0.2994 |
| 35 | CaMKIINalpha | −0.3896 | 0.0698 | 0.1268 | 0 |
| 36 | C16orf45 | −0.3465 | −0.0975 | 0.1899 | 0 |
| 37 | TCEAL1 | −0.3093 | −0.2368 | 0.2565 | −0.0257 |
| 38 | BLVRA | −0.2509 | 0.1475 | 0.0522 | −0.0533 |
| 39 | HIS1 | −0.1223 | −0.1352 | 0.1643 | −0.2116 |
| 40 | IGBP1 | −0.1384 | 0 | 0.0446 | 0.1256 |

FF PCR test (40 g × 35 s) Gene Scores (threshold = 0)

| | | | |
|---|---|---|---|
| Offset Quantile | 50 | Offset Value | 0.625782014 |
| | both | RNG Seed | 420473 |

| | | Prior Distribution (Sample Prior) | | | |
|---|---|---|---|---|---|
| | | BASAL | HER2 | LUMINAL | NORMAL-LIKE |
| | Class | | | | |
| | Prob. | 0.257142857 | 0.2 | 0.514285714 | 0.028571429 |
| | gene | | | | |
| 1 | KIT | 0.3675 | −0.1395 | −0.2345 | 1.8906 |
| 2 | ESR1 | −1.1883 | −1.3459 | 1.102 | 0.2805 |
| 3 | ID4 | 0.3245 | −0.2605 | −0.1333 | 1.3023 |
| 4 | FOXC1 | 0.9875 | −0.0291 | −0.5523 | 1.2582 |
| 5 | COX6C | −0.4466 | −0.3998 | 0.4409 | −1.1188 |
| 6 | SLC39A6 | −0.6537 | −0.959 | 0.761 | −1.1022 |
| 7 | ERBB2 | −0.4563 | 1.0711 | −0.1409 | −0.8546 |
| 8 | FABP7 | 1.0494 | −0.2077 | −0.4884 | 0.7995 |
| 9 | FZD7 | 0.2722 | −0.2176 | −0.1094 | 1.042 |
| 10 | CTPS | 0.5663 | 0.2286 | −0.3146 | −1.0343 |
| 11 | GRB7 | −0.1824 | 1.0102 | −0.2542 | −0.8547 |
| 12 | TFF3 | −0.9615 | −0.0486 | 0.5382 | −0.6947 |
| 13 | INPP4B | −0.617 | −0.2413 | 0.3534 | 0.8813 |
| 14 | FBP1 | −0.7295 | −0.1266 | 0.4627 | −0.8762 |
| 15 | XBP1 | −0.7415 | 0.1957 | 0.3423 | −0.8578 |
| 16 | B3GNT5 | 0.8518 | −0.0531 | −0.4234 | 0.3263 |
| 17 | KRT17 | 0.8451 | 0.5354 | −0.5876 | −0.7765 |
| 18 | GSTM3 | −0.4103 | −0.109 | 0.2041 | 0.7821 |
| 19 | GATA3 | −0.7014 | −0.7154 | 0.6473 | −0.3318 |
| 20 | HIS1 | −0.0594 | −0.0717 | 0.0968 | −0.7067 |
| 21 | GSTP1 | 0.7038 | −0.023 | −0.3381 | −0.0862 |
| 22 | TP53BP2 | 0.6448 | 0.2512 | −0.4122 | −0.1419 |
| 23 | CSDA | 0.6429 | 0.2468 | −0.4262 | 0.1579 |
| 24 | SLC5A6 | 0.6212 | 0.2941 | −0.4275 | 0.0468 |
| 25 | CDH3 | 0.5387 | 0.6139 | −0.5217 | 0.2459 |
| 26 | C16orf45 | −0.6045 | −0.3263 | 0.4143 | 0.2668 |
| 27 | BTG3 | 0.5543 | 0.5727 | −0.5099 | 0.1804 |
| 28 | FLJ10980 | −0.5708 | −0.1958 | 0.3594 | 0.0384 |
| 29 | SEMA3C | −0.5628 | 0.158 | 0.1883 | 0.5699 |
| 30 | TMSB10 | 0.3611 | 0.5673 | −0.3855 | −0.2823 |
| 31 | S100A11 | 0.5274 | 0.452 | −0.4164 | −0.415 |
| 32 | C10orf7 | 0.523 | 0.1684 | −0.3132 | −0.2481 |
| 33 | TCEAL1 | −0.4665 | −0.2879 | 0.3164 | 0.5185 |
| 34 | CHI3L2 | 0.5001 | −0.2528 | −0.142 | −0.1756 |
| 35 | VAV3 | −0.3937 | −0.0416 | 0.2301 | −0.3076 |
| 36 | CaMKIINalpha | −0.3558 | 0.237 | 0.0826 | 0.0551 |
| 37 | WWP1 | −0.3116 | −0.349 | 0.2975 | −0.1076 |
| 38 | BLVRA | −0.2769 | 0.2018 | 0.0711 | −0.2014 |
| 39 | ASF1A | 0.2576 | 0.1658 | −0.202 | 0.1569 |
| 40 | IGBP1 | −0.0697 | −0.0909 | 0.0723 | −0.0384 |

TABLE 17
Supplemental Table 4. Raw qRT-PCR (replicates averaged)
FFPE-PCR data 40 classifiers + 5 proliferation genes + 5 Housekeepers

| Matrix_SHK + | PCR Sample name | | | | | | |
|---|---|---|---|---|---|---|---|
| prolif_(45 g × 35 s) | BR00- | BR00- | BR00-0572_Int- | BR00- | BR99- | BR99- | IPB120- |
| Gene | 0284_FFPE | 0365_C_FFPE | VIM_FFPE | 0587_GATA3_FFPE | 0207_C_Int_FFPE | 0348 GATA3 | MET-LN_FFPE |
| Intrinsic gene list | | | | | | | |
| ASF1A_R1 | 0.6625 | 0.3485 | 1.27 | 0.467 | 0.28500003 | 0.3445 | 1.1800001 |
| B3GNT5_DR3 | 0.6835 | 0.542 | 6.5550003 | 0.861 | 0.838 | 1.3 | 8.035 |
| BLVRA_R2 | 2.1100001 | 2.96 | 0.6615 | 3.01 | 2.69 | 1.49 | 2.15 |
| BTG3_R1 | 1.905 | 0.87450004 | 3.29 | 0.6385 | 0.8345 | 2.31 | 3.545 |
| C10ORF7_R2 | 0.529 | 0.37449998 | 2.585 | 0.546 | 0.38050002 | 0.5325 | 1.655 |
| C16ORF45_R1 | 1.075 | 0.574 | 0.246 | 3.455 | 0.23949999 | 7.4449997 | 0.69949996 |
| CaMKIINalpha_R2 | 0.752 | 4.35 | 0.268 | 1.325 | 0.8355 | 4.14 | 1.0285 |
| CDH3_R1 | 0.5675 | 0.9015 | 2.31 | 0.142 | 0.127 | 1.06 | 6.08 |
| CHI3L2_R1 | 3.48 | 2.3175 | 268.5 | 2.575 | 29.15 | 14.1 | 108 |
| COX6C_R2 | 0.024599999 | 0.305 | 0.069 | 0.29500002 | 0.4435 | 0.8325 | 0.0995 |
| CSDA_R1 | 6.185 | 2.315 | 33.75 | 2.8000002 | 3.33 | 4.23 | 10.95 |
| CTPS_R1 | 0.2785 | 0.9785 | 0.81 | 0.43150002 | 0.6375 | 0.6625 | 2.96 |
| ERBB2_R5 | 3.685 | 1.195 | 0.62600005 | 0.97 | 1.325 | 0.899 | 0.6925 |
| ESR1_R3 | 0.2565 | 5.615 | 0.053729996 | 7.625 | 17.05 | 6.785 | 0.149 |
| FABP7_R2 | 0.002871 | 0.002871 | 0.118 | 0.002871 | 0.002871 | N/A | 0.007755 |
| FBP1_R1 | 0.4415 | 1.0999999 | 0.014249999 | 0.498 | 0.96349996 | 1.23 | 0.23699999 |
| FLJ10980_R2 | 1.9100001 | 0.3215 | 0.288 | 3.035 | 0.651 | 2.595 | 0.729 |
| FDXC1_DR2 | 5.19 | 3.595 | 90.1 | 1.9200001 | 1.71 | 4.99 | 31.099998 |
| FZD7_DR4 | 5.795 | 4.5 | 16.5 | 9.715 | 1.585 | 8.3500004 | 4.7 |
| GATA3_R4 | 0.2295 | 1.44 | 0.143 | 2.065 | 4.935 | 3.57 | 0.0501 |
| GRB7_DR4 | 2.66 | 0.7035 | 0.2125 | 0.64750004 | 0.336 | 0.906 | 0.32 |
| GSTM3_R3 | 0.21149999 | 0.5825 | 0.3195 | 1.2 | 0.1255 | 3.355 | 0.4765 |
| G5TP1_R2 | 5.605 | 3.7749999 | 27.95 | 3.79 | 4.2200003 | 15.1 | 24.2 |
| HIS1_DR1 | 1.88 | 3.845 | 0.988 | 2.4099998 | 3.375 | 6.44 | 3.335 |
| ID4_DR4 | 9.255 | 4.7749996 | 42.9 | 3.93 | 4.94 | 16.35 | 27.5 |
| IG3P1_R1 | 0.656 | 0.374 | 0.681 | 0.8815 | 0.372 | 0.538 | 0.6575 |
| INPP48_R1 | 1.065 | 1.24 | 0.15900001 | 1.034 | 0.4105 | 3.315 | 1.735 |
| KIT_R4 | 0.32599998 | 0.3745 | 1.0095 | 0.26200002 | 0.29000002 | 1.9300001 | 1.0475 |
| KRT17_R6 | 0.371 | 0.66550004 | 2.4850001 | 0.46100003 | 0.196 | 0.517 | 2.65 |
| S100A11_R5 | 0.9425 | 1.425 | 1.1700001 | 0.741 | 0.3215 | 1.5899999 | 1.6500001 |
| SEMA3C_R1 | 1.05 | 1.0295 | 0.5225 | 0.427 | 0.7885 | 0.7585 | 0.3755 |
| SLC39A6_DR3 | 0.04795 | 0.292 | 0.141 | 1.74 | 1.97 | 0.556 | 0.08335 |
| SLC5A6_DR4 | 0.5075 | 1.8900001 | 6.27 | 0.4365 | 0.64199996 | 2.01 | 1.605 |
| TCEAL1_DR3 | 0.683 | 2.1750002 | 1.635 | 1.433675 | 2.895 | 2.75 | 1.565 |
| TFF3_R2 | 1.175 | 35.35 | 0.5905 | 2.105 | 6.05 | 2.025 | 0.282 |
| TMSB10_R1 | 1.22 | 0.467 | 2.13 | 0.65999997 | 0.2365 | 0.5115 | 1.265 |
| TP53BP2_R1 | 1.46 | 2.31 | 16.1 | 0.833 | 0.9485 | 1.89 | 6.5600004 |
| VAV3_R1 | 1.7 | 7.8900003 | 1.3599999 | 6.815 | 0.12830001 | 5.2200003 | 1.2049999 |
| WWP1_R2 | 0.4345 | 5.695 | 0.371 | 0.8155 | 0.4075 | 4.515 | 0.85249996 |
| XBP1_R2 | 2.72 | 6.08 | 0.3595 | 4.5950003 | 3.275 | 4.95 | 1.28 |
| Proliferation genes | | | | | | | |
| BUB1_R3 | 0.382 | 0.8895 | 2.02 | 0.1995 | 0.3455 | 0.403 | 3.025 |
| MKI67_R4 | 0.2405 | 0.612 | 1.175 | 0.255 | 0.37449998 | 0.41000003 | 1.5999999 |
| MYBL2_R7 | 0.85899997 | 2 | 4.79 | 0.23699999 | 0.3725 | 0.39499998 | 4.725 |
| STK6_R5 | 0.13 | 0.4505 | 0.569 | 0.08515 | 0.14 | 0.1275 | 0.7825 |
| TOP2A_R7 | 0.21700001 | 0.249 | 0.2115 | 0.004185 | 0.0484 | 0.11 | 0.3735 |
| Housekeeper genes | | | | | | | |
| ACTB_R2 | 1.9649999 | 1.21 | 0.7185 | 1.1600001 | 1.04 | 1.7 | 2.415 |
| MRPL19_R2 | 2.225 | 3.335 | 5.575 | 2.6750002 | 2.885 | 2.63 | 5.02 |
| PSMC4_R3 | 0.16499999 | 0.4955 | 0.814 | 0.15 | 0.225 | 0.4115 | 0.5185 |
| PUM1_R6 | 1.24 | 0.649 | 1.28 | 1.17 | 0.6795 | 1.0450001 | 2.03 |
| SF3A1_R3 | 1.34 | 0.747 | 0.86300004 | 1.235 | 0.7705 | 0.96000004 | 1.815 |

| Matrix_SHK + | PCR Sample name | | | | | | | | | |
|---|---|---|---|---|---|---|---|---|---|---|
| prolif_(45 g × 35 s) | | | | | BR00- | BR00- | BR00- | BR99- | BR99- | PB120- |
| Gene | PB126_FFPE | PB149_FFPE | PB184_FFPE | BR00-0284 | 03655_C | 0572_Int-VIM | 0587_GATA3 | 0207_C_Int | 0348_GATA3 | MET-LN |
| Intrinsic gene list | | | | | | | | | | |
| ASF1A_R1 | 0.2085 | 0.949 | 0.089 | 11.95 | 3.725 | 1.24 | 3.255 | 6.27 | 4.58 | 4.0950003 |
| B3GNT5_DR3 | 4.3100004 | 1.75 | 1.315 | 4.31 | 1.21 | 1.625 | 1.345 | 1.685 | 3.3899999 | 15.4 |
| BLVRA_R2 | 0.1985 | 2.2150002 | 0.1285 | 31.85 | 18.4 | 0.39499998 | 11.1 | 26.45 | 16.900002 | 7.85 |
| BTG3_R1 | 2.7350001 | 1.345 | 1.28 | 19.95 | 2.73 | 2.355 | 1.8399999 | 4.84 | 10.085 | 9.49 |
| C10ORF7_R2 | 0.7095 | 0.7575 | 0.195 | 7.94 | 3.3600001 | 9.110001 | 3.5999998 | 5.495 | 4.5950003 | 5.635 |
| C16ORF45_R1 | 0.18180001 | 3.5149999 | 0.376 | 10.15 | 10.165 | 0.771 | 13.15 | 4.37 | 152.5 | 2.71 |
| CaMKIINalpha_R2 | 0.12450001 | 3.0700002 | 4.755 | 3.315 | 5.76 | 0.31800002 | 2.595 | 3.925 | 12.85 | 1.625 |
| CDH3_R1 | 4.09 | 1.185 | 0.64900005 | 8.6449995 | 2.5900002 | 1.0785 | 0.6365 | 0.5325 | 7.52 | 21.65 |
| CHI3L2_R1 | 643.5 | 15.1 | 13.65 | 153.5 | 6.265 | 2015 | 22.95 | 460.5 | 162 | 336.5 |
| COX6C_R2 | 0.016800001 | 0.056649998 | 0.01145 | 0.622 | 1.26 | 0.0402 | 2.685 | 6.88 | 9.96 | 0.5365 |
| CSDA_R1 | 10.8 | 10.8 | 1.725 | 16.5 | 4.79 | 65.2 | 1.5795 | 14.299999 | 10.950001 | 6.2200003 |
| CTPS_R1 | 1.56 | 0.87549996 | 0.22049999 | 3.04 | 2.255 | 1.155 | 1.064 | 4.9750004 | 5.3450003 | 9.434999 |
| ERBB2_R5 | 0.5395 | 0.854 | 0.885 | 28.05 | 2.58 | 0.187 | 2.16 | 4.26 | 4.33 | 2.1 |
| ESR1_R3 | 0.162 | 3.245 | 3.15 | 2.345 | 20.599998 | 0.06465 | 88.6 | 179 | 44.2 | 0.3445 |
| FABP7_R2 | 0.952 | 0.009945 | 0.00559 | 0.01145 | 0.003852 | 0.9575 | 0.0145 | 0.003852 | 0.003852 | 0.01118 |
| FBP1_R1 | 0.02985 | 0.5495 | 0.303 | 13.1 | 6.59 | 0.0352 | 5.035 | 13.2 | 10.7 | 0.7365 |
| FLJ10980_R2 | 0.1345 | 1.5799999 | 0.2995 | 39.6 | 6.83 | 1.5450001 | 25.099998 | 13.8 | 50.15 | 4.06 |
| FDXC1_DR2 | 43.2 | 7.765 | 3.245 | 17.6 | 3.085 | 23 | 2.24 | 2.0700002 | 6.415 | 36.85 |
| FZD7_DR4 | 5.33 | 13.950001 | 11.75 | 27.75 | 14.3 | 25.75 | 35.6 | 6.8050003 | 25.25 | 10.049999 |
| GATA3_R4 | 0.2995 | 1.9649999 | 1.525 | 3.77 | 4.345 | 0.035 | 15.700001 | 45.35 | 29.15 | 0.05835 |
| GRB7_DR4 | 1.2950001 | 0.29 | 0.6385 | 18.45 | 1.455 | 0.2145 | 1.48 | 1.465 | 5.4049997 | 1.34 |
| GSTM3_R3 | 0.08825 | 0.49 | 0.6595 | 5.46 | 6.51 | 2.295 | 21.400002 | 1.575 | 54.5 | 2.2 |
| G5TP1_R2 | 10.5 | 12.1 | 10.5 | 20.75 | 5.175 | 17.05 | 4.855 | 9.645 | 36.4 | 31.8 |
| HIS1_DR1 | 1.49 | 5.2200003 | 1.665 | 8.115 | 5.0550003 | 1.355 | 4.2349997 | 15.5 | 23.349998 | 5.455 |
| ID4_DR4 | 9.885 | 34.9 | 34.699997 | 35.05 | 25.599998 | 415 | 21.099998 | 17.5 | 83.8 | 63 |
| IG3P1_R1 | 0.1655 | 0.87049997 | 0.0765 | 13.6 | 5.2250004 | 0.9165 | 6.2799997 | 7.465 | 8.15 | 3.31 |
| INPP48_R1 | 0.0367 | 2.95 | 1.033 | 13.700001 | 7.125 | 0.1625 | 7.315 | 3.6399999 | 19.05 | 5.395 |
| KIT_R4 | 0.58000004 | 1.5150001 | 2.565 | 2.77 | 1.26 | 4.585 | 1.5150001 | 1.28 | 11.45 | 0.8555 |
| KRT17_R6 | 5.1099997 | 2.1399999 | 1.6 | 3.9899998 | 1.025 | 2.4650002 | 1.915 | 0.6885 | 1.175 | 2.05 |
| S100A11_R5 | 2.175 | 0.9505 | 1.315 | 6.285 | 1.575 | 0.873 | 1.9300001 | 1.33 | 4.495 | 5.5 |
| SEMA3C_R1 | 0.27899998 | 1.65 | 2.385 | 9.975 | 4.4049997 | 0.412 | 1.815 | 6.5550003 | 3.33 | 0.7355 |
| SLC39A6_DR3 | 0.0515 | 1.035 | 0.2585 | 0.93050003 | 0.94200003 | 0.14199999 | 9.88 | 16.45 | 4.7700005 | 0.31849998 |
| SLC5A6_DR4 | 4.705 | 1.3 | 0.8835 | 9.315001 | 6.525 | 19.05 | 1.45 | 4.105 | 12.6 | 5.96 |
| TCEAL1_DR3 | 0.832 | 1.38 | 4.825 | 11 | 8.639999 | 1.325 | 8.775 | 13.3 | 15.200001 | 6.5299997 |
| TFF3_R2 | 0.337 | 3.385 | 0.45200002 | 41.35 | 171.5 | 0.38300002 | 17.05 | 40.050003 | 13.55 | 0.77750003 |
| TMSB10_R1 | 1.2 | 0.8215 | 0.1155 | 18.25 | 4.76 | 0.977 | 2.835 | 3.715 | 6.27 | 10.6 |
| TP53BP2_R1 | 6.0649996 | 2.4099998 | 1.4200001 | 13.299999 | 6.4399996 | 7.745 | 2.335 | 5.2749996 | 9.315001 | 22.400002 |
| VAV3_R1 | 1.3 | 6.95 | 2.6999998 | 35.45 | 97.850006 | 2.3600001 | 82.15 | 3.885 | 80.4 | 4.27 |
| WWP1_R2 | 0.17050001 | 0.94449997 | 1.275 | 7.205 | 42.75 | 0.736 | 7.06 | 5.43 | 31.8 | 5.0599997 |
| XBP1_R2 | 0.437 | 3.055 | 4.135 | 40.8 | 26.95 | 0.151 | 40.35 | 20.6 | 24.85 | 2.165 |
| Proliferation genes | | | | | | | | | | |
| BUB1_R3 | 1.09 | 0.1615 | 0.1083 | 8.014999 | 3.395 | 5.755 | 1.49 | 2.025 | 1.695 | 13 |
| MKI67_R4 | 1.047 | 0.29500002 | 0.15 | 4.6 | 2.43 | 1.7850001 | 1.14 | 1.94 | 1.845 | 6.6 |
| MYBL2_R7 | 2.665 | 0.2715 | 0.0725 | 4.6549997 | 2.645 | 3.065 | 0.2655 | 0.519 | 0.624 | 6.415 |
| STK6_R5 | 0.4385 | 0.07080001 | 0.052500002 | 2.4099998 | 1.895 | 1.385 | 0.406 | 0.8295 | 0.5585 | 2.57 |
| TOP2A_R7 | 0.2395 | 0.035949998 | 0.00465 | 19.2 | 1.745 | 1.2 | 0.62 | 0.845 | 0.92649996 | 2.975 |
| Housekeeper genes | | | | | | | | | | |
| ACTB_R2 | 0.90400004 | 1.355 | 0.6815 | 15.45 | 3.37 | 0.8175 | 3.9250002 | 8.645 | 8.695 | 7.1499996 |
| MRPL19_R2 | 1.995 | 2.99 | 0.4555 | 14.85 | 5.37 | 1.1949999 | 3.625 | 7.4849997 | 6.6400003 | 10.094999 |
| PSMC4_R3 | 0.1325 | 0.19150001 | 0.048699997 | 4.1549997 | 3.595 | 1.175 | 2.45 | 4.6549997 | 3.7800002 | 5.0550003 |
| PUM1_R6 | 0.2685 | 1.74 | 0.188 | 21.5 | 10.7 | 9.18 | 9.184999 | 11.4 | 12.05 | 11.35 |
| SF3A1_R3 | 0.4105 | 1.985 | 0.29549998 | 17.15 | 6.55 | 1.5699999 | 7.42 | 12.1 | 9.525 | 8.465 |

FF-PCR data 40 classifiers + 5 proliferation genes + 5 Housekeepers

| Matrix_SHK + | PCR Sample name | | | | | | | | | | | |
|---|---|---|---|---|---|---|---|---|---|---|---|---|
| prolif_(45 g × 35 s) | | | | PB205T_{—} | PB255_{—} | PB297_{—} | PB311_{—} | PB314_{—} | PB334_{—} | PB362_{—} | PB370_{—} | PB376_{—} |
| Gene | PB126 | PB149 | PB184 | FFPE | FFPE | FFPE | FFPE | FFPE | FFPE | FFPE | FFPE | FFPE |
| Intrinsic gene list | | | | | | | | | | | | |
| ASF1A_R1 | 11.049999 | 1.58 | 10.450001 | 0.33850002 | 0.537 | 1.1500001 | 0.73800004 | 0.1163 | 0.519 | 0.14050001 | 0.24599999 | 1.505 |
| B3GNT5_DR3 | 39.55 | 1.545 | 8.085 | 5.635 | 3.0700002 | 5.925 | 1.6500001 | 1.175 | 6.14 | 0.6345 | 1.2 [symbol] | 4.83 |
| BLVRA_R2 | 8.715 | 3.94 | 4.085 | 0.417 | 1.755 | 0.668 | 3.15 | 1.085 | 0.99549997 | 0.5935 | 0.978 | 3.045 |
| BTG3_R1 | 31.15 | 2.7849998 | 10.6 | 4.99 | 2.51 | 6.995 | 1.62 | 1.15 | 4.255 | 1.155 | 1.0320001 | 3.26 |
| C10ORF7_R2 | 22 | 2.05 | 5.385 | 0.72749996 | 0.764 | 1.585 | 1.36 | 0.3395 | 2.455 | 0.176 | 0.363 | 1.19 |
| C16ORF45_R1 | 3.6299999 | 14.75 | 9.735 | 0.283 | 2.275 | 0.354 | 0.85150003 | 0.202 | 0.18100001 | 0.532 | 3.01 | 0.49 |
| CaMKIINalpha_R2 | 0.82 | 4.025 | 2.42 | 2.3899999 | 10.85 | 2.5900002 | 6.6459996 | 3.48 | 0.544 | 3.055 | 2.865 | 1.04 |
| CDH3_R1 | 154 | 8.825 | 14.85 | 1.0955 | 1.565 | 10.549999 | 2.52 | 1.56 | 1.625 | 0.2105 | 0.24849999 | 3.2649999 |
| CHI3L2_R1 | 6120 | 71.05 | 127 | 2.745 | 8.785 | 7605 | 156.5 | 2.79 | 550 | 32.3 | 14.85 | 22.5 |
| COX6C_R2 | 0.6795 | 0.174 | 0.4015 | 0.033150002 | 0.2805 | 0.107999995 | 0.85849996 | 0.01845 | 0.03415 | 0.0268 | 0.06425 | 0.0315 |
| CSDA_R1 | 57.850002 | 5.625 | 35.75 | 6.27 | 3.645 | 7.365 | 8.055 | 1.87 | 16.65 | 1.17 | 2.6999998 | 4.65 |
| CTPS_R1 | 15.799999 | 0.9515 | 10.5 | 2.21 | 0.6825 | 1.685 | 1.4100001 | 0.5185 | 2.3600001 | 0.2075 | 0.30900002 | 1.4300001 |
| ERBB2_R5 | 3.075 | 1.51 | 1.26 | 0.951 | 1.77 | 1.395 | 0.668 | 13.9 | 0.3315 | 0.614 | 1.45 | 0.49600002 |
| ESR1_R3 | 3.4 | 9.75 | 0.3955 | 0.053729996 | 3.585 | 0.11965 | 3.275 | 0.059698997 | 0.29500002 | 6.88 | 22.2 | 0.1 [symbol] 6 |
| FABP7_R2 | 2.065 | 0.09295 | 1.73 | 0.00589 | 0.002071 | 1.405 | 0.00 [symbol] 42 | 0.002871 | 0.17449999 | 0.002871 | 0.00526 | 0.01255 |
| FBP1_R1 | 0.22049999 | 1.345 | 0.8715 | 0.034649998 | 0.927 | 0.05485 | 0.579 | 0.1285 | 0.105900005 | 0.255 | 0.40850002 | 0.7705 |
| FLJ10980_R2 | 3.66 | 12.95 | 6.295 | 0.35750002 | 1.23 | 0.7365 | 0.4 [symbol] 85 | 0.31149998 | 0.37300003 | 0.228 | 1.31 | 0.5845 |
| FDXC1_DR2 | 93.6 | 5.475 | 55.599997 | 3.755 | 3.81 | 63 | 6.9700003 | 1.16 | 49.5 | 0.31649998 | 3.2 | 4.335 |
| FZD7_DR4 | 24.35 | 27.55 | 25.650002 | 4.575 | 14.6 | 17.9 | [symbol] .29 | 3.4250002 | 15.8 | 6.075 | 8.725 | 5.14 |
| GATA3_R4 | 2.21 | 7.025 | 0.0978 | 1.615 | 3.485 | 0.26749998 | 1.535 | 0.2235 | 0.100150004 | 1.905 | 1.745 | 0.1109 |
| GRB7_DR4 | 5.885 | 1.225 | 1.925 | 0.782 | 0.56949997 | 0.947 | 0.51600003 | 9.26 | 0.1815 | 0.3505 | 0.503 | 0.289 |
| GSTM3_R3 | 3.235 | 2.655 | 0.4105 | 0.2165 | 2 | 0.2805 | 1.53 | 0.07805 | 0.1745 | 1.5350001 | 1.375 | 0.192 |
| G5TP1_R2 | 57.5 | 10.585 | 35 | 46.95 | 17.45 | 37.1 | 10. [symbol] 85 | 3.2649999 | 34.9 | 2.655 | 4.99 | 30.05 |
| HIS1_DR1 | 20 | 6.66 | 11.35 | 6.075 | 6.975 | 4.3599997 | 3.085 | 1.44 | 4.455 | 1.365 | 5.925 | 4.985 |
| ID4_DR4 | 86.35 | 100.15 | 483 | 6.42 | 58.15 | 224.5 | 15.9 | 4.585 | 134 | 11.950001 | 14.700001 | 9.35 |
| IG3P1_R1 | 6.895 | 3.475 | 3.27 | 0. [symbol] 25 | 0.745 | 0.303 | 0.89100003 | 0.13550001 | 0.38599996 | 0.12449999 | 0.2935 | 0.85 |
| INPP48_R1 | 0.9835 | 10.55 | 2.145 | 0.199 | 3.455 | 0.363 | 4.1800003 | 0.15 | 0.31599998 | 2.135 | 2.81 | 0.63199997 |
| KIT_R4 | 11.3 | 5.3 | 6.095 | 0.2065 | 2.08 | 10.030001 | 0.49150002 | 0.6355 | 8.475 | 0.471 | 1.385 | 0.72650003 |
| KRT17_R6 | 21.75 | 6.275 | 42.25 | 1.575 | 1.38 | 3.2150002 | 8. [symbol] 45 | 1.2950001 | 52.699997 | 0.481 | 0.3565 | 2.65 |
| S100A11_R5 | 14.475 | 1.23 | 3.3899999 | 2.5549998 | 3.225 | 4.55 | 3.1950002 | 1. [symbol] 800001 | 2.605 | 1.275 | 1.205 | 1.1800001 |
| SEMA3C_R1 | 1.155 | 5.75 | 1.013 | 0.396 | 1.3050001 | 0.51750004 | 1.8399999 | 0.204 | 0.224 | 0.71650004 | 0.7985 | 1.26 |
| SLC39A6_DR3 | 0.82350004 | 2.555 | 0.74600005 | 0.0646 | 0.9415 | 0.23449999 | 0.2 [symbol] 65 | 0.041950002 | 0.09715 | 0.242 | 1.075 | 0.105000004 |
| SLC5A6_DR4 | 49.449997 | 3.28 | 7.6499996 | 6.225 | 3.095 | 2.52 | 1.8499999 | 2.255 | 2.59 | 1.535 | 1.31 | 1.575 |
| TCEAL1_DR3 | 5.26 | 7.635 | 5.33 | 1.3535 | 9.8 [symbol] 2501 | 1.655 | 3.1 | 0.71850004 | 0.7285 | 1.665 | 1.6269999 | 1.7745001 |
| TFF3_R2 | 2.185 | 9.315001 | 2.04 | 0.429 | 8.31 | 0.90900004 | 9. [symbol] 35 | 0.40350002 | 0.4305 | 1.305 | 7.61 | 1.485 |
| TMSB10_R1 | 25.55 | 2.19 | 8.395 | 0.774 | 0.934 | 1.62 | 2.2800002 | 0.212 | 1.165 | 0.1115 | 0.321 | 1.4 |
| TP53BP2_R1 | 54.2 | 3.2350001 | 9.055 | 3.22 | 2.665 | 27.35 | 3.114999 [symbol] | 1.81 | 4.45 | 3.0149999 | 2 | 2.245 |
| VAV3_R1 | 73.5 | 39.65 | 5.365 | 0.90749997 | 9.235001 | 1.99 | 2.8049998 | 1.0665 | 0.597 | 1.3199999 | 2.705 | 3.045 |
| WWP1_R2 | 2.46 | 3.105 | 4.27 | 0.64199996 | 2.295 | 1.4 | 1.245 | 0.605 | 0.89849997 | 1.0550001 | 1.6949999 | 0.704 |
| XBP1_R2 | 1.685 | 9.26 | 2.76 | 0.5995 | 12 | 1.135 | 12.05 | 3.495 | 1.645 | 2.7849998 | 9.309999 | 3.175 |
| Proliferation genes | | | | | | | | | | | | |
| BUB1_R3 | 23.1 | 0.5825 | 11.1 | 2.7350001 | 0.87399995 | 2.3200002 | 1. [symbol] 15 | 0.6 | 1.4449999 | 0.357 | 0.24649999 | 0.7985 |
| MKI67_R4 | 13.6 | 0.629 | 12.25 | 4.17 | 0.8835 | 1.385 | 1.1 | 0.594 | 2.3899999 | 0.32099998 | 0.5615 | 1.65 |
| MYBL2_R7 | 8.684999 | 0.2665 | 7.4849997 | 10.9 | 1.48 | 4.41 | 1.985 | 1.2750001 | 8.16 | 0.6465 | 0.7945 | 1.8 |
| STK6_R5 | 6.955 | 0.1875 | 4.265 | 3.8 | 1.105 | 0.7175 | 0. [symbol] 49 | 0.1745 | 0.822 | 0.1595 | 0.131 | 0.36900002 |
| TOP2A_R7 | 18.4 | 0.125 | 5.1800003 | 1.095 | 0.2705 | 0.9 | 0. [symbol] 82 | 1.56 | 0.7575 | 0.135 | 0.004185 | 0.282 |
| Housekeeper genes | | | | | | | | | | | | |
| ACTB_R2 | 12.8 | 2.355 | 8.53 | 1.555 | 2.12 | 2.29 | 2.665 | 0.62049997 | 1.14 | 0.5445 | 0.6555 | 1.16 |
| MRPL19_R2 | 18 | 3.0149999 | 4.635 | 1.985 | 1.915 | 4.4849997 | 4.39 | 0.91550004 | 4.205 | 0.7355 | 1.1800001 | 3.0749998 |
| PSMC4_R3 | 8.565 | 0.6475 | 9.49 | 0.24149999 | 0.2415 | 0.41750002 | 0.51100004 | 0.08335 | 0.2625 | 0.055600002 | 0.1125 | 0.14 |
| PUM1_R6 | 15.049999 | 7.99 | 11.799999 | 0.6115 | 0.62450004 | 2.275 | 1.715 | 0.289 | 0.8995 | 0.44050002 | 0.272 | 0.85249996 |
| SF3A1_R3 | 11.4 | 4.915 | 17.95 | 0.662 | 0.6185 | 2.0149999 | 1.675 | 0.3095 | 1.0799999 | 0.4765 | 0.379 | 0.9735 |

| Matrix_SHK + | PCR Sample name | | | | | | | | | | | |
|---|---|---|---|---|---|---|---|---|---|---|---|---|
| prolif_(45 g × 35 s) | PB379_{—} | PB413_{—} | PB441_{—} | PB455_{—} | UB29_1C_{—} | | | | | | | |
| Gene | FFPE | FFPE | FFPE | FFPE | FFPE | PB205T | PB255 | PB297 | PB311 | PB314 | PB334 | PB362 |
| Intrinsic gene list | | | | | | | | | | | | |
| ASF1A_R1 | 0.08535 | 0.6705 | 1.78 | 0.741 | 1.91 | 3.72 | 6.285 | 10.355 | 5.465 | 2.085 | 5.86 | 2.79 |
| B3GNT5_DR3 | 8.08 | 1.985 | 1.69 | 2.83 | 19.4 | 4.255 | 1.96 | 11.35 | 1.855 | 0.935 | 20.150002 | 2.495 |
| BLVRA_R2 | 0.314 | 2.31 | 6.69 | 2.85 | 2.4050002 | 3.4099998 | 14.450001 | 3.1750002 | 12.9 | 7.925 | 8.775 | 5.4399996 |
| BTG3_R1 | 2.15 | 1.375 | 2.32 | 4.59 | 7.855 | 7.565 | 6.21 | 35.75 | 6.915 | 16.05 | 15.645 | 4.125 |
| C10ORF7_R2 | 0.291 | 1.045 | 0.93850005 | 0.82 | 3.38 | 3.885 | 6.5550003 | 11.700001 | 8.915001 | 5.565 | 32.2 | 2.56 |
| C16ORF45_R1 | 0.18180001 | 1.05 | 3.155 | 1.205 | 0.6935 | 2.145 | 34.05 | 4.335 | 5.965 | 5.435 | 3.2199998 | 10.635 |
| CaMKIINalpha_R2 | 0.8985 | 1.6700001 | 5.34 | 3.24 | 2.1 | 2.795 | 29.25 | 7.48 | 30.7 | 21.25 | 2.1000001 | 4.09 |
| CDH3_R1 | 1.22 | 0.81299996 | 0.3085 | 5.31 | 11.05 | 3.021 | 4.5150003 | 35.75 | 6.915 | 16.05 | 15.645 | 4.125 |
| CHI3L2_R1 | 122.5 | 29 | 250.5 | 13.9 | 943 | 3.845 | 123 | 35450 | 286 | 66.25 | 1615 | 59.050003 |
| COX6C_R2 | 0.0838 | 1.165 | 0.53999996 | 0.042400002 | 0.07665 | 0.175 | 5.485 | 1.035 | 5.745 | 0.5565 | 0.77699995 | 0.14649999 |
| CSDA_R1 | 1.78 | 3.48 | 1.935 | 4.4350004 | 12.15 | 7.975 | 12.95 | 39.25 | 23.55 | 12.6 | 43.25 | 6.4300003 |
| CTPS_R1 | 0.2015 | 0.56149995 | 0.8255 | 1.84 | 1.4849999 | 8.360001 | 3.51 | 8.595 | 6.225 | 5.6800003 | 14.799999 | 0.8915 |
| ERBB2_R5 | 0.39450002 | 1.135 | 1.4200001 | 35.85 | 1.21 | 0.7705 | 8.255 | 2.76 | 1.2065 | 35.15 | 1.0265 | 0.6115 |
| ESR1_R3 | 0.99399996 | 33.25 | 47.7 | 0.264 | 0.373 | 0.193 | 47.75 | 0.5525 | 17.6 | 1.12 | 3.505 | 10.059999 |
| FABP7_R2 | 0.002871 | 0.047399998 | 0.002871 | 0.00473 | 5.95 | 0.0303 | 0.007445 | 17.95 | 0.00484 | 0.004915 | 1.4 | 0.0436 |
| FBP1_R1 | 0.10349999 | 2.5549998 | 2.2 | 1.1800001 | 0.533 | 0.168 | 7.74 | 0.3475 | 2.2 | 1.78 | 1.49 | 0.796 |
| FLJ10980_R2 | 0.5395 | 1.78 | 3.9650002 | 1.185 | 0.7655 | 2.5149993 | 32.25 | 11.9 | 5.4449997 | 13.450001 | 11.25 | 9.395 |
| FDXC1_DR2 | 3.53 | 5.625 | 5.9300003 | 9.47 | 98.9 | 2.345 | 2.13 | 98.5 | 4.605 | 1.355 | 57.8 | 10.6 |
| FZD7_DR4 | 3.04 | 9.105 | 13.75 | 22.849996 | 18.05 | 8.014999 | 25.8 | 66.85 | 11.25 | 18.400002 | 94.1 | 34.25 |
| GATA3_R4 | 0.407 | 9.49 | 4.4 | 0.5855 | 1.1 | 2.545 | 21.05 | 1.56 | 9.205 | 5.26 | 1.37 | 2.8400002 |
| GRB7_DR4 | 0.2095 | 0.43449998 | 0.469 | 26.45 | 1.12 | 1.255 | 4.77 | 3.13 | 1.97 | 77.25 | 4.325 | 0.729 |
| GSTM3_R3 | 0.4705 | 0.2195 | 2.085 | 3.87 | 0.22749999 | 0.814 | 29.85 | 4.04 | 9.365 | 1.9649999 | 2.12 | 9.450001 |
| G5TP1_R2 | 19.849998 | 27.3 | 18.45 | 11.3 | 41.25 | 37.4 | 12.950001 | 64.5 | 5.17 | 2.995 | 26.9 | 6.76 |
| HIS1_DR1 | 1.21 | 4.84 | 9.36 | 3.3200002 | 3.395 | 8.675 | 22.15 | 13.55 | 10.4 | 9.700001 | 12.8 | 3.7649999 |
| ID4_DR4 | 8.184999 | 73.55 | 12.2 | 27.4 | 21.4 | 6.5550003 | 220 | 399.5 | 19.25 | 14.950001 | 159.5 | 145.5 |
| IG3P1_R1 | 0.0676 | 1.016 | 3.16 | 0.85800004 | 0.76699996 | 3.365 | 8.235001 | 2.185 | 5.4049997 | 4.7650003 | 7.29 | 3.585 |
| INPP48_R1 | 0.261 | 1.735 | 7.83 | 1. [symbol] 15 | 1.3299999 | 0.3255 | 12.45 | 1.2650001 | 14 | 1.4300001 | 1.85 | 10.025 |
| KIT_R4 | 2.27 | 3.7150002 | 1.0125 | 7.17 | 5.14 | 0.7635 | 8.475 | 38.8 | 1.235 | 5.115 | 66.65 | 22.75 |
| KRT17_R6 | 8.535 | 2.12 | 0.5475 | 1.425 | 7.2650003 | 2.64 | 1.4200001 | 5.1049995 | 7.415 | 5.5299997 | 49.75 | 7.8 |
| S100A11_R5 | 1.425 | 1.5550001 | 3.395 | 4.4 | 11.24 | 4.715 | 2.6799998 | 16.55 | 10.55 | 7.425 | 18 | 1.725 |
| SEMA3C_R1 | 0.465 | 0. [symbol] 9049995 | 1.815 | 1.4649999 | 1.525 | 0.509 | 4.99 | 1.3 | 4.825 | 2.4 | 0.6605 | 4.0699997 |
| SLC39A6_DR3 | 0.261 | 0.722 | 3.405 | 0.44099998 | 0.155 | 0.156 | 9.129999 | 1.29 | 0.7575 | 0.6045 | 1.225 | 0.36 |
| SLC5A6_DR4 | 0.37150002 | 0.744 | 1.092 | 2.0549998 | 6.8199997 | 7.665 | 8.02 | 12.45 | 6.5950003 | 12.200001 | 22.05 | 3.6 |
| TCEAL1_DR3 | 2.605 | 6.84 | 4.875 | 3.165 | 2.73 | 2.1799998 | 24.55 | 4.25 | 7.42 | 3.06 | 2.795 | 8.09 |
| TFF3_R2 | 0.5105 | 9.565001 | 44.8 | 20 | 0.7475 | 2.6 | 107 | 2.24 | 38.6 | 25.25 | 1.405 | 5.42 |
| TMSB10_R1 | 0.1245 | 0.479 | 0.627 | 1.0550001 | 2.33 | 6.225 | 6.855 | 17.2 | 13.4 | 6.4300003 | 13.1 | 2.96 |
| TP53BP2_R1 | 1.765 | 2.58 | 3.805 | 7.755 | 7.135 | 3.355 | 5.51 | 77.4 | 5.5 [symbol] 99997 | 7.455 | 20.2 | 3.7450001 |
| VAV3_R1 | 1.0545 | 3.97 | 16 | 2.345 | 2.325 | 13.1 | 260.5 | 25.45 | 66.2 | 115 | 22.7 | 28.3 |
| WWP1_R2 | 0.6185 | 1.9 | 2.885 | 1.095 | 0.90999997 | 1.22 | 10.35 | 9.105 | 6.535 | 4.67 | 6.6549997 | 3.8400002 |
| XBP1_R2 | 9.285 | 14.1 | 26.900002 | 11.45 | 2.12 | 0.7985 | 34.1 | 2.6399999 | 25.349998 | 11.55 | 8.73 | 3.12 |
| Proliferation genes | | | | | | | | | | | | |
| BUB1_R3 | 0.6455 | 0.8225 | 0.4895 | 0.845 | 2.155 | 5.125 | 2.84 | 15.85 | 5.74 | 3.43 | 7.3050003 | 0.62549996 |
| MKI67_R4 | 0.324 | 0.748 | 0.4685 | 0.95449996 | 1.5550001 | 4.295 | 2.535 | 9.2 | 4.0150003 | 1.925 | 10.450001 | 0.5715 |
| MYBL2_R7 | 1.1099999 | 0.667 | 0.2595 | 0.724 | 7.035 | 7.21 | 1.0139999 | 5.045 | 2.78 | 2.065 | 6.8050003 | 0.299 |
| STK6_R5 | 0.0878 | 0.26099998 | 0.167 | 0.252 | 1.025 | 9.025 | 5.465 | 3.795 | 2.05 | 1.125 | 4.205 | 0.17750001 |
| TOP2A_R7 | 0.0343 | 0.21000001 | 0.174 | 1.15 | 0.5115 | 2.415 | 2.065 | 10.200001 | 2.58 | 20.05 | 6.955 | 0.149 |
| Housekeeper genes | | | | | | | | | | | | |
| ACTB_R2 | 0.949 | 1.62 | 1.96 | 4.06 | 4.58 | 5.3900003 | 10.125 | 9.82 | 9.635 | 7.6 | 13.55 | 3.3249998 |
| MRPL19_R2 | 0.56299995 | 3.21 | 8.74 | 3.89 | 6.3999996 | 3.975 | 4.41 | 6.285 | 6.2550003 | 3.405 | 9.93 | 2.52 |
| PSMC4_R3 | 0.142 | 0.4325 | 0.7155 | 0.6165 | 0.94299996 | 3.7450001 | 4.1549997 | 5.28 | 7.255 | 4.45 | 8.76 | 1.1099999 |
| PUM1_R6 | 0.244 | 1.1800001 | 2.395 | 1.095 | 2.27 | 5.445 | 17.45 | 25.25 | 14.450001 | 12.2 | 21.55 | 8.13 |
| SF3A1_R3 | 0.4145 | 0.96099997 | 1.55 | 1.15 | 2.034999 [symbol] | 6.425 | 13 | 14.65 | 10.275 | 4.5649996 | 14.950001 | 6.215 |

| Matrix_SHK + | PCR Sample name | | | | | | | | | | |
|---|---|---|---|---|---|---|---|---|---|---|---|
| prolif_(45 g × 35 s) | | | | | | | | UB37_7V_{—} | UB38_1D_{—} | UB39_5I_{—} | UB43_4B_{—} |
| Gene | PB370 | PB376 | PB379 | PB413 | PB441 | PB455 | UB29 | FFPE | FFPE | FFPE | FFPE |
| Intrinsic gene list | | | | | | | | | | | |
| ASF1A_R1 | 26.5 | 9.105 | 2.58 | 1.74 | 3.75 | 0.4525 | 4.41 | 0.268 | 0.9395 | 1.28 | 0.1805 |
| B3GNT5_DR3 | 17.5 | 10.530001 | 3.105 | 0.5425 | 1.6700001 | 0.1915 | 8.92 | 1.79 | 4.295 | 1.605 | 1.2375 |
| BLVRA_R2 | 79.7 | 16.6 | 9.425 | 5.41 | 13.15 | 2.22 | 3.21 | 1.345 | 4.8900003 | 4.9 | 0.66499996 |
| BTG3_R1 | 31.1 | 22 | 3.48 | 1.94 | 2.9450002 | 1.3900001 | 11.3 | 1.965 | 2.01 | 1.885 | 1.0345 |
| C10ORF7_R2 | 36.35 | 12.95 | 3.4099998 | 2.725 | 2.8000002 | 0.723 | 7.6 | 0.4855 | 1.115 | 1.245 | 0.3395 |
| C16ORF45_R1 | 455 | 5.3599997 | 3.67 | 10.13 | 11.29 | 6.34 | 1.9100001 | 0.3085 | 3.725 | 1.165 | 0.18180001 |
| CaMKIINalpha_R2 | 98 | 4.935 | 4.49 | 2.615 | 3.79 | 1.655 | 1.645 | 1.007 | 3.93 | 9.139999 | 1.905 |
| CDH3_R1 | 38.95 | 58.1 | 1.6700001 | 1.5150001 | 0.513 | 1.8 | 9.87 | 3.7 | 1.1949999 | 3.13 | 1.425 |
| CHI3L2_R1 | 415.5 | 250.5 | 560 | 93.45 | 382 | 4.3199997 | 1560 | 2.3175 | 79.75 | 166.5 | 42.8 |
| COX6C_R2 | 21.15 | 0.468 | 3.28 | 1.9300001 | 1.27 | 0.019749999 | 0.19 | 0.0296 | 0.3115 | 0.925 | 0.225 |
| CSDA_R1 | 53 | 10.4 | 8.645 | 5.455 | 4.8500004 | 1.9849999 | 29 | 2.7849998 | 8.345 | 3.82 | 0.339 |
| CTPS_R1 | 36.6 | 9.32 | 1.81 | 2.3400002 | 2.325 | 1.59 | 2.8449998 | 0.6025 | 0.71000004 | 1.34 | 0.324 |
| ERBB2_R5 | 16.65 | 3.1100001 | 1.765 | 1.0525 | 1.26 | 12.014999 | 0.376 | 17 | 1.385 | 5.105 | 0.3515 |
| ESR1_R3 | 653 | 0.9755 | 19 | 43.55 | 70.1 | 0.07635 | 0.216 | 0.0881 | 9.450001 | 19.7 | 2.23 |
| FABP7_R2 | 0.0431 | 0.123500004 | 0.003852 | 0.08135 | 0.003852 | 0.003852 | 8.945 | 0.002871 | 0.01315 | 0.007085 | 0.00319 |
| FBP1_R1 | 48.6 | 5.19 | 1.26 | 6.235 | 3.205 | 0.527 | 0.4035 | 0.0704 | 1.8199999 | 2.75 | 0.57449996 |
| FLJ10980_R2 | 103 | 13.55 | 20.05 | 17.1 | 18.6 | 2.6599998 | 4.865 | 0.277 | 3.4850001 | 3.005 | 0.366 |
| FDXC1_DR2 | 19.95 | 3.755 | 3.4099998 | 2.83 | 2.205 | 1.2195001 | 45.5 | 2.53 | 14.450001 | 9.665 | 1.14 |
| FZD7_DR4 | 220.5 | 17.45 | 4.42 | 6.455 | 15.5 | 3.916 | 21.2 | 6.245 | 14.3 | 14.6 | 5.965 |
| GATA3_R4 | 79 | 1.92 | 3.94 | 12.35 | 7.575 | 0.52 | 1.62 | 0.08505 | 3.225 | 15.75 | 1.795 |
| GRB7_DR4 | 25.95 | 2.965 | 1.96 | 0.0815 | 1.0285001 | 15.549999 | 0.979 | 9.344999 | 0.78 | 3.56 | 0.2165 |
| GSTM3_R3 | 244 | 4.285 | 10.549999 | 0.4615 | 6.575 | 8.035 | 0.5255 | 0.4409 | 0.913 | 2.3000002 | 0.093150005 |
| G5TP1_R2 | 70.1 | 60.3 | 14.75 | 14.549999 | 4.36 | 2.08 | 13.6 | 8.965 | 30.150002 | 17.7 | 7.715 |
| HIS1_DR1 | 88 | 27 | 12.1 | 10.9 | 17.5 | 2.375 | 3.085 | 2.395 | 8.17 | 9.264999 | 0.5765 |
| ID4_DR4 | 272.5 | 11.950001 | 10.9 | 179.5 | 21.5 | 8.9 | 32.1 | 32.85 | 45.800003 | 20.2 | 15.25 |
| IG3P1_R1 | 39.6 | 5.83 | 4.305 | 3.46 | 7.37 | 0.6545 | 1.715 | 0.27899998 | 1.89 | 1.225 | 0.2175 |
| INPP48_R1 | 204 | 2.12 | 0.9165 | 2.67 | 11.549999 | 0.65250003 | 1.97 | 0.2945 | 2.5300002 | 7.245 | 2.13 |
| KIT_R4 | 50.5 | 4.7200003 | 2.895 | 3.205 | 1.345 | 2.955 | 7.755 | 0.612 | 6.855 | 2.995 | 1.23 |
| KRT17_R6 | 20.099998 | 18.599998 | 0.20899999 | 2.045 | 0.5865 | 0.333 | 6.245 | 1.11 | 2.8400002 | 8.51 | 0.7365 |
| S100A11_R5 | 31.5 | 8.73 | 1.765 | 0.5955 | 3.685 | 1.0699999 | 5.205 | 2.145 | 2.265 | 1.49 | 1.175 |
| SEMA3C_R1 | 54.7 | 9.065001 | 0.9805 | 0.77849996 | 3.135 | 0.3435 | 0.57449996 | 0.7935 | 2.1399999 | 3.025 | 1.79 |
| SLC39A6_DR3 | 29 | 0.4385 | 4.215 | 1.99 | 4.3599997 | 0.13 | 0.1445 | 0.115 | 1.7950001 | 2.68 | 0.25550002 |
| SLC5A6_DR4 | 42.75 | 11.049999 | 1.7850001 | 1.635 | 2.4499998 | 1.69 | 11.25 | 1.23 | 1.705 | 0.90550005 | 1.11 |
| TCEAL1_DR3 | 90.1 | 11.5 | 9.565001 | 15.6 | 5.84 | 1.56 | 3.385 | 1.1800001 | 5.105 | 7.74 | 1.6099999 |
| TFF3_R2 | 153.5 | 5.97 | 5.0299997 | 19.45 | 51.8 | 17.1 | 0.4125 | 0.4775 | 41.65 | 42.9 | 5.8599997 |
| TMSB10_R1 | 54.550003 | 21.5 | 2.145 | 1.395 | 3.35 | 1.9399999 | 5.83 | 1.115 | 1.7950001 | 1.27 | 0.48049998 |
| TP53BP2_R1 | 29.5 | 12.55 | 4.25 | 2.585 | 4.7200003 | 2.645 | 5.16 | 2.5149999 | 5.1850004 | 2.92 | 1.435 |
| VAV3_R1 | 845 | 147 | 131.5 | 43 | 84.95 | 9.174999 | 20 | 2.3600001 | 12.05 | 4.45 | 0.235 |
| WWP1_R2 | 58.95 | 4.7749996 | 7.205 | 4.1549997 | 7.025 | 0.4305 | 1.29 | 0.4275 | 2.69 | 4.27 | 1.105 |
| XBP1_R2 | 122.5 | 14.6 | 25.1 | 7.8 | 36.6 | 2.13 | 1.0235 | 1.72 | 10.6 | 14.5 | 2.355 |
| Proliferation genes | | | | | | | | | | | |
| BUB1_R3 | 20.5 | 7.17 | 2.175 | 1.25 | 0.94200003 | 0.6385 | 3.23 | 0.4795 | 0.6305 | 0.92149997 | 0.662 |
| MKI67_R4 | 27.05 | 9.34 | 0.98500003 | 1.095 | 0.7195 | 0.52 | 3.105 | 0.33 | 0.668 | 0.9235 | 0.98350005 |
| MYBL2_R7 | 11.450001 | 4.6949997 | 1.505 | 0.3645 | 0.2335 | 0.3615 | 4.385 | 1.4549999 | 0.549 | 1.15 | 1.2850001 |
| STK6_R5 | 7.875 | 3.0749998 | 0.3655 | 0.372 | 0.301 | 0.176 | 1.5350001 | 0.176 | 0.24450001 | 0.521 | 0.25849998 |
| TOP2A_R7 | 11.9 | 4.005 | 0.22850001 | 0.3885 | 0.3375 | 0.867 | 0.913 | 0.004185 | 0.134 | 0.418 | 0.113000005 |
| Housekeeper genes | | | | | | | | | | | |
| ACTB_R2 | 73.55 | 10.799999 | 3.16 | 1.95 | 3.39 | 2.605 | 5.545 | 1.435 | 3.125 | 1.54 | 2.065 |
| MRPL19_R2 | 30.150002 | 8.115 | 2.05 | 2.33 | 3.29 | 1.1555 | 2.79 | 1.855 | 5.615 | 3.125 | 2.205 |
| PSMC4_R3 | 33.05 | 4.5550003 | 3.375 | 1.0799999 | 2.775 | 0.495 | 1.665 | 0.31 | 0.75549996 | 0.729 | 0.2685 |
| PUM1_R6 | 113 | 16.150002 | 6.075 | 5.8450003 | 9.46 | 1.575 | 7.6000004 | 0.55550003 | 2.22 | 1.61 | 0.5185 |
| SF3A1_R3 | 69.65 | 11.5 | 5.52 | 2.565 | 7.7250004 | 1.125 | 3.57 | 0.4425 | 2.64 | 1.4749999 | 0.72099996 |

| Matrix_SHK + | | | | | | | | | |
|---|---|---|---|---|---|---|---|---|---|
| prolif_(45 g × 35 s) | PCR Sample name | | | | | | | | |
| Gene | UB45_6D_FFPE | UB55_5D_FFPE | UB57_3D_FFPE | UB58_7E_FFPE | UB60_3D_FFPE | UB66_1D_FFPE | UB37 | UB38 | UB39 |
| Intrinsic gene list | | | | | | | | | |
| ASF1A_R1 | 0.01845 | 0.0633 | 0.15349999 | 0.164 | 2.15 | 0.078 | 4.67 | 6.8149996 | 4.755 |
| B3GNT5_DR3 | 1.0455 | 1.605 | 0.9 | 0.865 | 2.865 | 0.9555 | 6.73 | 5.74 | 2.2649999 |
| BLVRA_R2 | 0.00733 | 0.276 | 0.488 | 0.58000004 | 7.01 | 0.479 | 16.65 | 14.2 | 12.8 |
| BTG3_R1 | 3.06 | 0.83449996 | 1.345 | 0.7475 | 5.375 | 2.335 | 8.59 | 3.6399999 | 3.555 |
| C10ORF7_R2 | 0.07805 | 0.566 | 0.2845 | 0.52349997 | 1.775 | 0.26999998 | 5.08 | 2.84 | 4.18 |
| C16ORF45_R1 | 0.18180001 | 3.04 | 0.62549996 | 0.7075 | 1.6949999 | 0.18180001 | 10.92 | 28.55 | 9.175 |
| CaMKIINalpha_R2 | 1.39 | 6.495 | 1.58 | 2.1 | 5.5699997 | 1.175 | 5.75 | 6.005 | 10.715 |
| CDH3_R1 | 1.095 | 0.2495 | 0.08095 | 0.5145 | 4.575 | 0.273 | 26.45 | 2.49 | 3.645 |
| CHI3L2_R1 | 264 | 42.5 | 12.275 | 8.5 | 43.4 | 87.35 | 6.54 | 574.5 | 415 |
| COX6C_R2 | 0.0284 | 0.02795 | 0.2615 | 0.089200005 | 0.1125 | 0.2105 | 0.6685 | 1.895 | 3.9899998 |
| CSDA_R1 | 0.0174 | 2.1 | 3.0149999 | 1.34 | 5.42 | 0.275 | 11.15 | 8.715 | 6.595 |
| CTPS_R1 | 0.023850001 | 0.2805 | 0.5095 | 0.18149999 | 1.38 | 0.208 | 5.945 | 3.145 | 5.7799997 |
| ERBB2_R5 | 2.185 | 1.435 | 0.704 | 0.7135 | 23 | 1.51 | 50.6 | 2.3400002 | 4.705 |
| ESR1_R3 | 17.95 | 6.3500004 | 4.825 | 3.145 | 0.553 | 25.3 | 0.7985 | 55.1 | 48.5 |
| FABP7_R2 | 0.002871 | 0.002871 | 0.00746 | 0.002871 | 0.02895 | 0.002871 | 0.003852 | 0.085975 | 0.00428 |
| FBP1_R1 | 0.11570001 | 0.5405 | 0.8655 | 0.22 | 1.033 | 0.2515 | 0.82299995 | 6.5249996 | 7.0550003 |
| FLJ10980_R2 | 0.1621 | 1.98 | 0.635 | 1.89 | 4.5150003 | 0.737 | 8.94 | 41 | 24.05 |
| FDXC1_DR2 | 0.41750002 | 1.565 | 2.54 | 0.9785 | 9.13 | 0.875 | 3.61 | 4.205 | 3.135 |
| FZD7_DR4 | 14.15 | 10.3 | 8.555 | 4.925 | 12.95 | 10.33 | 31.900002 | 16.85 | 15.799999 |
| GATA3_R4 | 1.85 | 3.5949998 | 5.7349997 | 1.0450001 | 0.5345 | 4.74 | 0.754 | 23.900002 | 33.4 |
| GRB7_DR4 | 2.5500002 | 0.96599996 | 0.969 | 0.294 | 9.35 | 1.07 | 57.6 | 1.665 | 7.2349997 |
| GSTM3_R3 | 0.03655 | 3.045 | 0.1895 | 1.0699999 | 0.505 | 0.988 | 4.365 | 5.1850004 | 14.35 |
| G5TP1_R2 | 5.705 | 10.200001 | 5.09 | 4.315 | 50.7 | 14.549999 | 15.45 | 23.2 | 5.67 |
| HIS1_DR1 | 0.5145 | 4.185 | 2.505 | 1.625 | 8.095 | 1.7850001 | 10.6 | 22.650002 | 26.849998 |
| ID4_DR4 | 11 | 8.375 | 6.365 | 7.64 | 46.35 | 23.349998 | 224.5 | 43.45 | 36.55 |
| IG3P1_R1 | 0.0288 | 0.112 | 0.22299999 | 0.182 | 2.48 | 0.312 | 4.3500004 | 11.2 | 7.34 |
| INPP48_R1 | 1.54 | 3.6599998 | 0.5875 | 1.12 | 5.3149996 | 1.54 | 2.9 | 12.15 | 14.299999 |
| KIT_R4 | 0.518 | 2.355 | 0.315 | 1.385 | 3.9299998 | 0.47 | 4.32 | 5.625 | 2.3200002 |
| KRT17_R6 | 0.702 | 0.486 | 0.3085 | 0.94 | 10.45 | 0.17639999 | 2.3449998 | 0.799 | 10.25 |
| S100A11_R5 | 2.385 | 2.85 | 0.621 | 1.1 | 6.075 | 1.565 | 6.9049997 | 4.0699997 | 1.5150001 |
| SEMA3C_R1 | 5.74 | 2.58 | 0.8355 | 1.525 | 2.6399999 | 3.585 | 6.21 | 5.5299997 | 5.19 |
| SLC39A6_DR3 | 3.5149999 | 1.7 | 0.512 | 0.6955 | 0.176 | 0.98599994 | 8.62049997 | 5.755 | 7.7349997 |
| SLC5A6_DR4 | 3.14 | 1.29 | 0.64849997 | 0.75 | 5.15 | 0.8725 | 7.04 | 3.25 | 1.855 |
| TCEAL1_DR3 | 3.03775 | 5.79 | 6.115 | 1.66 | 4.285 | 4.8775 | 5.98 | 21.599998 | 16.45 |
| TFF3_R2 | 0.31849998 | 1.006 | 62.35 | 15.55 | 9.82 | 12.5 | 4.575 | 390.5 | 187.5 |
| TMSB10_R1 | 0.0396 | 0.201 | 0.226 | 0.44 | 6.4449997 | 0.1415 | 15.8 | 8.555 | 4.065 |
| TP53BP2_R1 | 2.795 | 4.475 | 1.1 | 1.4300001 | 8.26 | 1.455 | 12.8 | 18.4 | 4.2200003 |
| VAV3_R1 | 0.4255 | 9.110001 | 1.825 | 8.945 | 8.13 | 0.845 | 81.2 | 108 | 60.2 |
| WWP1_R2 | 0.8405 | 1.9949999 | 0.398 | 0.7585 | 1.975 | 1.5450001 | 5.325 | 13.7 | 14.15 |
| XBP1_R2 | 6.675 | 7.625 | 10.235001 | 1.4 | 8.799999 | 17.95 | 7.725 | 31.75 | 16.5 |
| Proliferation genes | | | | | | | | | |
| BUB1_R3 | 0.46850002 | 0.503 | 0.271 | 0.252 | 0.854 | 0.542 | 3.225 | 1.02 | 1.6500001 |
| MKI67_R4 | 0.3595 | 0.5045 | 0.3266 | 0.2495 | 0.66349995 | 0.4395 | 2.225 | 1.25 | 1.315 |
| MYBL2_R7 | 0.758 | 1.1800001 | 0.34100002 | 0.342 | 1.0999999 | 0.2035 | 2.985 | 0.3015 | 0.50699997 |
| STK6_R5 | 0.19999999 | 0.2855 | 0.117 | 0.1275 | 0.286 | 0.217 | 1.0799999 | 0.538 | 1.035 |
| TOP2A_R7 | 0.004185 | 0.26749998 | 0.0674 | 0.059699997 | 0.09445 | 0.004185 | 0.6055 | 0.4745 | 1.017 |
| Housekeeper genes | | | | | | | | | |
| ACTB_R2 | 0.5985 | 1.0215 | 0.559 | 0.677 | 5.24 | 0.68850005 | 12.1 | 5.6949997 | 3.5900002 |
| MRPL19_R2 | 0.305 | 0.5935 | 1.0799999 | 0.6175 | 7.62 | 1.255 | 7.635 | 6.955 | 2.615 |
| PSMC4_R3 | 0.04485 | 0.15200001 | 0.1285 | 0.2335 | 1.145 | 0.121999994 | 7.415 | 4.03 | 2.99 |
| PUM1_R6 | 0.2659 | 0.347 | 0.32349998 | 0.7125 | 2.6399999 | 0.2135 | 10.25 | 7.8199997 | 11.45 |
| SF3A1_R3 | 0.184 | 0.385 | 0.563 | 0.35549998 | 2.435 | 0.2995 | 8.355 | 10.264999 | 4.85 |

| Matrix_SHK + | | | | | | | |
|---|---|---|---|---|---|---|---|
| prolif_(45 g × 35 s) | PCR Sample name | | | | | | |
| Gene | UB43 | UB45 | UB55 | UB578 | UB58 | UB60A | UB66 |
| Intrinsic gene list | | | | | | | |
| ASF1A_R1 | 2.415 | 4.80 | 0.7085 | 4.63 | 4.9049997 | 7.135 | 8.73 |
| B3GNT5_DR3 | 1.38 | 3.185 | 0.9235 | 5.5299997 | 4.9750004 | 6.18 | 4.84 |
| BLVRA_R2 | 21 | 14.3 | 3.74 | 6.0699997 | 18.75 | 15.95 | 85.75 |
| BTG3_R1 | 4.6400003 | 13.85 | 0.347 | 0.25335 | 9.76 | 14.86 | 19.8 |
| C10ORF7_R2 | 6.99 | 5.02 | 1.47 | 5.1800003 | 5.8 | 6.25 | 20.099998 |
| C16ORF45_R1 | 15.1 | 166.5 | 33.85 | 24.95 | 17.099998 | 13.434999 | 111 |
| CaMKIINalpha_R2 | 3.44 | 9.42 | 15.5 | 5.8450003 | 20.7 | 14.299999 | 1.0799999 |
| CDH3_R1 | 8.559999 | 5.7 | 0.347 | 0.25335 | 9.76 | 14.86 | 3.09 |
| CHI3L2_R1 | 73.45 | 500 | 183.5 | 101.7 | 66.4 | 106.6 | 49.65 |
| COX6C_R2 | 2.805 | 1.705 | 0.1645 | 7.7200003 | 1.075 | 0.42650002 | 5.72 |
| CSDA_R1 | 6.5950003 | 10.55 | 2.01 | 10.9 | 12.75 | 9.34 | 3.08 |
| CTPS_R1 | 5.135 | 4.455 | 0.975 | 3.9499998 | 3.37 | 3.7 | 2.705 |
| ERBB2_R5 | 2.185 | 5.525 | 0.97099996 | 0.985 | 4.02 | 36.050003 | 1.53 |
| ESR1_R3 | 14.15 | 65.95 | 23.55 | 58 | 33.65 | 3.01 | 234.5 |
| FABP7_R2 | 0.007535 | 0.00972 | 0.003852 | 0.00513 | 0.01087 | 0.07665 | 0.003852 |
| FBP1_R1 | 5.21 | 4.31 | 1.89 | 8.059999 | 4.1800003 | 2.645 | 9.959999 |
| FLJ10980_R2 | 14.75 | 53.75 | 10.65 | 14.200001 | 28.8 | 25 | 106.5 |
| FDXC1_DR2 | 1.7349999 | 1.63 | 1.2195001 | 4.6400003 | 3.3049998 | 7.68 | 2.74 |
| FZD7_DR4 | 17.55 | 31.099998 | 9.97 | 22 | 57.15 | 19.1 | 73.8 |
| GATA3_R4 | 9.94 | 11.305 | 7.775 | 38.35 | 20.45 | 1.175 | 18.7 |
| GRB7_DR4 | 1.335 | 11.200001 | 0.9325 | 2.33 | 4.38 | 20.900002 | 5.295 |
| GSTM3_R3 | 3.08 | 3.5100002 | 11.35 | 3.2150002 | 18.900002 | 1.235 | 19.85 |
| G5TP1_R2 | 8.46 | 4.7650003 | 2.195 | 4.215 | 16.3 | 18.1 | 25.75 |
| HIS1_DR1 | 9.235001 | 19.05 | 4.91 | 17.9 | 22.2 | 13.05 | 10.75 |
| ID4_DR4 | 37.9 | 30.25 | 3.5749998 | 19.2 | 56.35 | 84.3 | 130.5 |
| IG3P1_R1 | 4.9700003 | 17.55 | 3.295 | 9.139999 | 7.4049997 | 8.24 | 45.15 |
| INPP48_R1 | 6.585 | 28.8 | 6.475 | 3.435 | 13.85 | 9.48 | 40 |
| KIT_R4 | 3.59 | 3.8650002 | 1.815 | 2.1799998 | 9.805 | 10.9 | 5.495 |
| KRT17_R6 | 4.88 | 2.7649999 | 0.1795 | 0.376 | 12.049999 | 9.94 | 0.116 |
| S100A11_R5 | 3.9899998 | 12.7 | 2.73 | 2.2350001 | 10.645 | 9.365 | 6.165 |
| SEMA3C_R1 | 7.24 | 27.75 | 2.525 | 2.505 | 7.3999996 | 5.2200003 | 18.7 |
| SLC39A6_DR3 | 1.78 | 10.75 | 3.4499998 | 4.355 | 12.9 | 0.5205 | 5.875 |
| SLC5A6_DR4 | 4.735 | 11.1 | 1.51 | 1.5699999 | 7.1099997 | 8.485001 | 5.4049997 |
| TCEAL1_DR3 | 6.6499996 | 14 | 5.3999996 | 21.650002 | 9.585 | 8.275 | 54.05 |
| TFF3_R2 | 140.5 | 38.75 | 5.4449997 | 458.5 | 385.5 | 67.100006 | 220.5 |
| TMSB10_R1 | 14.5 | 8.035 | 1.065 | 3.24 | 6.7 | 12.6 | 12.1 |
| TP53BP2_R1 | 5.025 | 12.65 | 3.22 | 2.935 | 7.835 | 11.15 | 4.9700003 |
| VAV3_R1 | 84.649994 | 224.5 | 104 | 70.45 | 264.5 | 64.85 | 62.449997 |
| WWP1_R2 | 5.91 | 16.65 | 4.025 | 3.745 | 10.2 | 6.5150003 | 27.85 |
| XBP1_R2 | 9.025 | 26.150002 | 5.285 | 39.75 | 8.51 | 13.35 | N/A |
| Proliferation genes | | | | | | | |
| BUB1_R3 | 0.978 | 8.175 | 1.235 | 1.19 | 3.3200002 | 3 | 4.495 |
| MKI67_R4 | 0.7865 | 1.335 | 0.84749997 | 0.963 | 3.26 | 2.82 | 3.5549998 |
| MYBL2_R7 | 0.86950004 | 0.784 | 0.6625 | 0.269 | 0.998 | 1.245 | 0.3635 |
| STK6_R5 | 0.5625 | 0.6925 | 0.54700005 | 0.4525 | 1.26 | 1.115 | 1.36 |
| TOP2A_R7 | 0.228 | 0.6175 | 0.5415 | 0.4925 | 2 | 0.57449996 | 2.6750002 |
| Housekeeper genes | | | | | | | |
| ACTB_R2 | 15.21 | 10.95 | 3.095 | 5.365 | 12.04999 | 9.275 | 18.35 |
| MRPL19_R2 | 2.925 | 3.44 | 1.575 | 3.675 | 5.6 | 3.76 | 4.26 |
| PSMC4_R3 | 3.565 | 5.665 | 1.905 | 5.23 | 5.555 | 5.505 | 11.1 |
| PUM1_R6 | 7.495 | 16 | 5.45 | 11.25 | 17.25 | 11.35 | 33.75 |
| SF3A1_R3 | 9.545 | 13.55 | 2.4 | 15.15 | 11.275 | 8.85 | 32.949997 |

[symbol]

indicates data missing or illegible when filed

TABLE 18

| | | Sample | | | | | | | | | | | | |
|---|---|---|---|---|---|---|---|---|---|---|---|---|---|---|
| | | 9830-0100 | BC00030- [symbol] | 030222-M [symbol] | 030064-B [symbol] | 030521-B [symbol] | 9819-0003 | BC00051 | BC00063 | BC00087 | 9819-0003 | 020573-B [symbol] | 020577-M [symbol] | BC00036T |
| | | Class | | | | | | | | | | | | |
| Symbol | NAME | HER2 | HER2 | HER2 | HER2 | HER2 | HER2 | HER2 | HER2 | HER2 | HER2 | HER2 | HER2 | |
| | | −0.5095 | −1.6805 | −0.8795 | 0.3315 | 0.8155 | −1.4065 | 0.7935 | −0.5885 | 0.6935 | −2.1725 | −2.6325 | −0.4235 | −1.3755 |
| | | −0.05 | −0.181 | −0.033 | 0.629 | 0.397 | 0.378 | −0.092 | 0.038 | −0.464 | −0.516 | −0.217 | 0.043 | −0.394 |
| ABAT | 882378 \|\| [symbol] | 3.772 | −0.14 | 0.339 | 2.224 | 2.212 | 2.622 | 3.35 | 1.699 | 0.818 | 0.426 | 1.156 | 1.242 | 2.857 |
| ABC1 | 696882 \|\| [symbol] | 1.9595 | 1.0445 | 0.2205 | −1.4215 | 1.7615 | 0.2765 | 0.6815 | 1.4915 | 0.8115 | −0.8035 | 0.5125 | 0.5665 | 1.8355 |
| ABCC11 | 697619 \|\| [symbol] | −0.4705 | −0.8825 | −1.2975 | −0.9205 | 0.6255 | 1.9945 | −1.3925 | 0.7995 | 0.8925 | 0.9165 | −1.5215 | −0.3195 | −0.3145 |
| ABCC3 | 695262 \|\| [symbol] | −0.105 | −0.065 | −0.812 | −0.916 | −1.204 | −0.62 | −1.293 | −0.236 | −0.152 | 0.534 | 0.82 | 0.348 | 0.726 |
| ACAS2L | 695930 \|\| [symbol] | −0.0055 | 0.2625 | −0.7405 | −0.3895 | 0.0415 | −0.5675 | 1.0675 | −0.5905 | 0.8495 | 1.2635 | 1.0695 | 1.4025 | 0.6525 |
| ACAT1 | 686983 \|\| [symbol] | 0.55 | 0.175 | −0.164 | 2.796 | 0.388 | 1.202 | 2.132 | 0.333 | −1.467 | 1.064 | 0.301 | 2.28 | −0.777 |
| ACAT2 | 691318 \|\| [symbol] | 0.2215 | 0.3765 | −0.2855 | −0.3005 | −0.3835 | 0.0105 | 0.6455 | −0.8195 | −0.1275 | 1.2845 | 0.4195 | 0.6025 | −0.0395 |
| ACOX2 | 694388 \|\| [symbol] | −0.1305 | −0.2105 | −0.8885 | 0.2615 | 0.1765 | −0.9445 | 1.3655 | −0.1835 | 0.3805 | 0.5875 | −0.3345 | −0.4025 | −1.0605 |
| ACDX3 | 690904 \|\| [symbol] | −0.3555 | 0.1535 | 0.1205 | 0.0665 | −0.0795 | −0.1055 | −0.4935 | 0.2945 | −0.0215 | −0.4225 | 0.3185 | −0.4235 | 0.6025 |
| ACSL3 | 690853 \|\| [symbol] | −0.201 | 0.64 | 0.581 | 0.596 | 0.211 | 0.641 | 1.816 | 0.812 | 0.343 | −0.068 | 0.297 | 0.845 | 0.5 |
| ACTA1 | 692913 \|\| [symbol] | 0.0845 | 0.0475 | 0.6265 | 0.3445 | 0.0135 | −0.1045 | 0.0645 | 0.1305 | −0.0125 | 0.0705 | 0.0925 | 0.4305 | 0.3795 |
| ACTB | 695048 \|\| [symbol] | 0.8695 | 0.4595 | 0.9445 | 0.6535 | −0.2335 | 0.3325 | 1.1315 | 1.1675 | 0.1255 | 1.1715 | 1.1025 | 1.9415 | −0.3485 |
| ADAM33 | 682670 \|\| [symbol] | 2.645 | 0.826 | −0.705 | −1.38 | −0.124 | −1.094 | −0.126 | −0.773 | −0.955 | 2.281 | 0.63 | 2.35 | −0.445 |
| ADAMDE [symbol] | 687861 \|\| [symbol] | 0.6435 | 0.1905 | −1.2665 | 0.4745 | 1.3535 | −0.0075 | 0.6195 | −0.4395 | −0.6205 | 0.2145 | −0.0995 | −0.0875 | −0.5045 |
| ADPN | 684643 \|\| [symbol] | 0.9815 | 1.3475 | −0.2075 | −0.5825 | 0.7585 | 1.8165 | 0.3245 | 1.1835 | 0.3165 | 1.6535 | 0.0305 | −0.3295 | −0.2655 |
| ADPRHL1 | 681147 \|\| [symbol] | 0.4565 | 1.6055 | 0.3295 | −0.3275 | −0.4815 | 1.8365 | −3.5785 | −0.5715 | 2.1695 | −3.1775 | 1.2055 | 2.1805 | −0.0185 |
| ADRA2C | 696803 \|\| [symbol] | 2.3895 | 3.0755 | 2.2775 | 1.7755 | 0.8405 | −0.7785 | −0.1535 | 0.7935 | −0.1795 | 0.0595 | 0.2895 | 0.7475 | −0.4115 |
| AGR2 | 696132 \|\| [symbol] | 0.1785 | 0.0105 | −0.1255 | 0.1805 | −0.0275 | −0.2905 | −0.2575 | −0.1785 | −0.1375 | −0.1585 | −0.2735 | −0.1195 | 0.1475 |
| AGT | 685839 \|\| [symbol] | 0.015 | 0.033 | −0.945 | 0.5 | 0.393 | −0.239 | 0.298 | −0.002 | −0.889 | −0.382 | −0.469 | −0.443 | −0.055 |
| AGTR1 | 683209 \|\| [symbol] | 1.552 | −0.448 | 0.657 | −0.288 | −0.177 | −0.21 | −0.890 | −0.068 | −0.742 | −1.281 | −0.365 | 0.175 | 0.399 |
| AHCYL1 | 681920 \|\| [symbol] | −0.131 | 1.771 | 0.287 | −1.823 | −0.283 | −0.574 | −0.31 | −0.209 | −0.302 | 0.519 | 0.493 | 0.753 | −0.204 |
| AHNAK | 696607 \|\| [symbol] | −0.082 | −1.061 | −0.591 | −0.613 | −0.365 | 0.85 | 0.428 | −1.087 | −0.549 | −0.012 | 0.757 | 1.3 | 1.521 |
| AHR | 687282 \|\| [symbol] | −0.1435 | 0.3885 | 0.2065 | 0.5205 | −0.4865 | −0.4255 | 0.5055 | −0.4295 | −0.0025 | 0.8225 | 0.4205 | 0.6315 | −0.0065 |
| AIM1 | 692581 \|\| [symbol] | −0.1825 | 0.8765 | −1.1285 | −1.2245 | −1.7625 | −0.1985 | −0.6725 | −0.2045 | 0.1285 | 0.3735 | 0.3035 | −0.1055 | −0.6255 |
| AK2 | 686272 \|\| [symbol] | 1.9155 | 0.5865 | −0.6075 | 0.3015 | 0.9475 | −0.4015 | −0.2035 | −0.2515 | −0.1375 | −1.9485 | 0.0455 | −0.1975 | −1.5195 |
| AK3L1 | 689094 \|\| [symbol] | −0.425 | −0.4 | −0.085 | −0.28 | −0.931 | −0.237 | −0.267 | −1.018 | −1.429 | 1.095 | 0.138 | 0 | −0.126 |
| AK5 | 686869 \|\| [symbol] | 0.535 | 1.48 | −0.684 | 0.746 | 1.32 | 0.01 | 1.325 | 0.245 | 0.193 | 1.742 | 0.003 | −0.23 | 0.357 |
| AKAP1 | 689352 \|\| [symbol] | −0.2025 | 0.0795 | −0.7775 | 0.8115 | −0.0155 | 0.1485 | −0.4315 | −0.2045 | −0.4545 | −0.2715 | 0.3355 | 1.0475 | 0.1625 |
| AKR1A1 | 681581 \|\| [symbol] | 0.241 | 0.323 | −0.135 | −1.025 | −0.257 | −0.658 | −0.006 | −0.312 | −0.289 | 0.488 | 0.25 | −0.038 | 0.131 |
| AKR7A3 | 685301 \|\| [symbol] | −0.4895 | 0.0315 | 0.2185 | 0.7475 | −0.2965 | −0.3795 | −0.5815 | 0.0925 | 0.0815 | 0.5575 | 0.4975 | 0.8645 | 0.4425 |
| ALG5 | 686176 \|\| [symbol] | 0.3415 | 1.6285 | −0.5665 | −0.0015 | 0.1625 | 0.5525 | 0.5745 | 0.2825 | 0.9065 | 0.4475 | −0.0195 | 0.6355 | −0.7725 |
| ALG8 | 681787 \|\| [symbol] | 0.2665 | 0.4655 | −0.1575 | −0.5545 | −1.2255 | −0.1925 | −1.1205 | 0.5165 | 0.0975 | −0.4685 | −0.1225 | −0.3265 | 0.0225 |
| AMFR | 693971 \|\| [symbol] | −0.511 | −0.665 | −0.114 | 0.31 | 0.244 | 0.202 | 0.396 | 0.281 | −0.612 | 0.105 | −0.294 | 9.112 | −0.23 |
| AMPD2 | 683330 \|\| [symbol] | −0.045 | 1.010 | 1.288 | 0.702 | 0.333 | 1.083 | 0.158 | 0.452 | 0.195 | 1.123 | −0.894 | 0.598 | −0.477 |
| ANAPC11 | 684913 \|\| [symbol] | −0.005 | 0.018 | 3.151 | −0.918 | −0.734 | 0.656 | 0.318 | 0.47 | 0.723 | 0.636 | −0.430 | −0.955 | 0.314 |
| ANKRD22 | 691041 \|\| [symbol] | 0.753 | 0.253 | 0.886 | −1.219 | −0.504 | 0.784 | 1.738 | 0.023 | 0.68 | 0.42 | 1.274 | −0.298 | 0.968 |
| ANKRD27 | 693340 \|\| [symbol] | −0.207 | −0.334 | −0.138 | −0.315 | 1.894 | −0.226 | 0.292 | 0.096 | −0.047 | −0.025 | −0.069 | −0.282 | −0.519 |
| ANLN | 687252 \|\| [symbol] | 0.302 | 0.266 | −2.803 | −3.609 | 0.88 | −1.558 | −0.014 | −0.678 | 0.013 | −0.929 | 0.341 | 0.115 | −1.055 |
| ANP32A | 886123 \|\| [symbol] | 0.378 | −0.261 | 0.3 | −0.127 | −0.6 | −0.08 | 0.531 | 1.193 | 0.675 | 0.146 | −0.108 | −0.001 | −0.801 |
| ANXA3 | 688113 \|\| [symbol] | −0.6625 | 0.6235 | 0.4135 | 0.2625 | 1.1885 | 1.1965 | −2.4855 | 0.6475 | 1.1075 | 0.9525 | 1.1965 | 0.9395 | 0.0295 |
| APBB2 | 688899 \|\| [symbol] | 0.0885 | −1.0895 | −0.9315 | −0.5125 | −0.3645 | −0.6335 | −1.2525 | 0.5375 | −0.4225 | −1.2845 | 1.7825 | 0.8615 | 3.8095 |
| APOBEC3 | 691683 \|\| [symbol] | 0.081 | −0.17 | −1.946 | 2.077 | 2.145 | 0.699 | 1.298 | 0.168 | 0.402 | 0.922 | −0.017 | −0.358 | 1.329 |
| AQP5 | 681185 \|\| [symbol] | −1.299 | −1.183 | 2.295 | −1.098 | 1.943 | −1.872 | −0.41 | 2.715 | 2.218 | −1.37 | −0.312 | 2.048 | −2.128 |
| AR | 893848 \|\| [symbol] | 0.1315 | −0.2875 | 0.1375 | −0.2635 | 0.1185 | 0.6735 | 1.8955 | 0.0475 | 1.0695 | −0.3335 | 0.2885 | 0.1695 | −0.2215 |
| AREG | 896877 \|\| [symbol] | −0.242 | −0.227 | −0.218 | 0.662 | 0.27 | −0.19 | −0.385 | 0.197 | 0.364 | −0.781 | −0.217 | 0.595 | −0.66 |
| ARF1 | 694438 \|\| [symbol] | −0.8245 | 0.4335 | 0.2795 | 0.1245 | 0.3235 | −0.0755 | 0.8945 | 0.2025 | −0.1595 | −0.6715 | −0.2385 | −0.2445 | 0.0735 |
| ARHGDI [symbol] | 694908 \|\| [symbol] | 0.301 | 0.771 | −1.254 | −0.431 | −0.021 | 0.486 | 0.709 | −0.23 | −0.014 | −0.454 | −0.138 | −0.508 | −0.409 |
| ARL6IP | 688270 \|\| [symbol] | −0.1305 | −0.2925 | −2.1555 | −1.0395 | −0.0285 | −0.6195 | −0.6465 | 0.1155 | 0.0345 | −2.1145 | −0.6505 | −0.3955 | −1.7185 |
| ARL6IP2 | 884285 \|\| [symbol] | −1.827 | −0.834 | −1.105 | −0.24 | −1.665 | −0.459 | −1.208 | −1.19 | 0.489 | −2.471 | −1.06 | 0.963 | −2.344 |
| ARMCX2 | 882195 \|\| [symbol] | −2.066 | −0.877 | −0.788 | −0.328 | −1.791 | −0.518 | −1.404 | −1.037 | 0.388 | −2.501 | −0.94 | 0.947 | −2.527 |
| ARNT2 | 885876 \|\| [symbol] | −0.541 | 0.364 | −0.89 | −0.888 | −0.13 | −0.409 | 0.052 | −0.637 | −0.39 | −0.075 | −0.141 | −0.8 | −0.551 |
| ARNT2 | 686537 \|\| [symbol] | 0.7375 | 0.8945 | −0.7115 | −2.0905 | 0.3105 | −0.1355 | 1.6295 | −0.3595 | −0.2625 | 0.6295 | 0.3385 | 0.1295 | −0.2275 |
| ARP3BET [symbol] | 896804 \|\| [symbol] | 0.5475 | −0.1245 | 0.1025 | 0.6525 | 0.3145 | 0.7465 | 0.6275 | 0.1985 | 0.4855 | −0.3055 | 0.4705 | 0.5015 | 0.4465 |
| ARPC1B | 895647 \|\| [symbol] | 0.005 | 1.368 | −0.072 | 0.205 | 0.208 | −0.009 | −0.696 | 0.56 | 2.425 | −0.276 | 0.588 | 0.891 | −0.684 |
| ARPC5L | 890357 \|\| [symbol] | 0.2155 | 0.5505 | −1.3205 | −0.5885 | 0.5355 | 0.6595 | 0.1685 | −0.3135 | 0.2685 | 0.2245 | 0.4475 | 0.5505 | 1.6455 |
| AS89 | 885235 \|\| [symbol] | −0.51 | −0.214 | −0.681 | −0.303 | 0.202 | −0.266 | 0.488 | −0.05 | 0.406 | −0.312 | 0.286 | 0.085 | −0.834 |
| ASF1A | 692334 \|\| [symbol] | −0.0705 | −0.1875 | −0.4975 | −1.1875 | −0.5815 | 1.8525 | 1.3615 | −1.1525 | 0.2445 | 0.2685 | 0.3175 | −0.3775 | 0.8185 |
| ASH2L | 696184 \|\| [symbol] | −0.533 | −0.541 | 0.43 | −0.505 | −0.448 | 0.692 | 0.909 | 0.263 | 0.647 | −0.293 | 0.704 | −0.083 | 0.603 |
| ASK | 686886 \|\| [symbol] | 1.482 | 1.228 | −0.467 | 1.731 | 1.675 | −0.755 | 1.828 | 0.143 | 0.35 | 0.302 | 1.9 | 1.129 | 0.969 |
| ASPM | 695669 \|\| [symbol] | −1.1945 | 0.4045 | −0.1055 | −0.9855 | −0.7245 | 0.3295 | 0.5415 | −0.3855 | −0.8335 | 0.8275 | 1.0805 | 0.8545 | 0.9925 |
| ASS | 695913 \|\| [symbol] | 0.6245 | −0.2225 | 0.7225 | −0.0315 | −0.6315 | 1.0545 | −0.1785 | 0.1495 | 0.7125 | 0.0525 | 0.1485 | 1.1645 | 1.1935 |
| ATAD2 | 682321 \|\| [symbol] | 0.1835 | 1.2935 | −1.1955 | 0.3145 | −1.1645 | 0.3805 | 0.6695 | 0.4375 | −0.4935 | 0.5785 | −0.0005 | −0.4115 | 0.0585 |
| ATF5 | 684294 \|\| [symbol] | 0.331 | 0.311 | 0.18 | −0.465 | 1.448 | −0.003 | −0.914 | 0.167 | 0.355 | −0.122 | −0.308 | −0.739 | 0.042 |
| ATF7IP2 | 681599 \|\| [symbol] | −0.341 | −0.269 | 1.73 | −0.014 | −1.083 | 1.886 | −0.494 | −0.859 | −0.33 | 0.668 | −0.253 | −0.393 | 2.402 |
| ATP2B1 | 685531 \|\| [symbol] | 0.112 | 0.198 | −0.367 | 0.499 | −0.194 | 0.077 | 0.607 | −0.03 | 0.285 | 1.111 | −0.001 | −0.25 | 0.039 |
| ATP5G1 | 695474 \|\| [symbol] | 0.2195 | −0.0175 | 0.4455 | 0.7695 | 0.1105 | 0.1695 | 0.4535 | 0.0285 | 0.3955 | −0.2625 | −0.1735 | 0.3645 | −0.0275 |
| ATP5O | 896801 \|\| [symbol] | 0.434 | 1.167 | −0.383 | 0.448 | 3.429 | 0.136 | 1.751 | −0.732 | −0.393 | 3.44 | −0.059 | −0.947 | 2.315 |
| ATP6AP1 | 681830 \|\| [symbol] | −0.313 | −0.719 | −0.242 | 0.125 | −0.566 | −0.682 | −0.102 | −0.818 | 0.492 | 0.321 | −0.889 | −0.443 | −0.594 |
| ATP6V0A4 | 881594 \|\| [symbol] | 0.554 | −0.278 | 0.069 | 0.411 | 0.71 | 0.492 | 0.576 | −0.147 | 0.535 | −0.988 | −0.239 | −0.027 | −0.293 |
| ATP6V1B [symbol] | 692388 \|\| [symbol] | 0.2815 | 0.1885 | 0.8955 | 0.6075 | −0.9775 | −0.5295 | 0.6015 | −0.4775 | −0.2265 | 0.4415 | −0.1195 | −0.4335 | 0.1485 |
| ATP6V1G [symbol] | 696310 \|\| [symbol] | −0.215 | 0.314 | −0.37 | −0.403 | −0.681 | −0.374 | −0.58 | 0.388 | −0.381 | 0.248 | 0.188 | 0.221 | −0.942 |
| ATPIF1 | 692140 \|\| [symbol] | 0.3145 | 2.1245 | 0.0405 | −1.2405 | 1.0305 | 2.2175 | 2.6805 | 0.0915 | −0.9345 | −1.0185 | 0.5045 | −1.1885 | −1.7585 |
| ATXN10 | 685606 \|\| [symbol] | 0.199 | 0.781 | 0.464 | 0.835 | −0.076 | −0.699 | 1.026 | 0.518 | −0.336 | 1.044 | 0.209 | 0.047 | 1.047 |
| B3GNT5 | 683718 \|\| [symbol] | −0.5685 | −1.0445 | −0.1135 | −1.3505 | −1.7265 | −1.3115 | −1.6705 | −0.7725 | 1.2015 | −0.5455 | −0.1555 | −0.1775 | 0.2615 |
| BACE2 | 687028 \|\| [symbol] | −0.2655 | 0.0235 | −0.1575 | −0.2405 | −0.3605 | −0.1045 | 0.1915 | −0.0035 | 0.1775 | −0.0025 | 0.4855 | 0.3125 | −0.6275 |
| BAG1 | 888260 \|\| [symbol] | −1.2455 | −0.2735 | 1.2455 | −0.0585 | −0.6645 | −0.9545 | −0.4275 | −0.5745 | −0.4345 | −0.9515 | −0.6265 | −0.4105 | −0.5585 |
| BAG4 | 889183 \|\| [symbol] | 0.0355 | 0.0695 | −0.4175 | 2.0025 | 0.7375 | −0.7625 | 2.6145 | −1.3745 | −1.7975 | −0.4505 | 0.6875 | 2.0585 | −0.4705 |
| BAI2 | 684125 \|\| [symbol] | 0.029 | −0.587 | 1.755 | 0.281 | −0.401 | 1.202 | −0.048 | −0.127 | 1.35 | −0.006 | −0.193 | 0.7 | 0.072 |
| BAMBI | 686435 \|\| [symbol] | −0.437 | −0.56 | −0.062 | 0.026 | −0.212 | −0.324 | −0.004 | −0.155 | −0.251 | −0.192 | −0.023 | 0.883 | −0.202 |
| BCAS1 | 886280 \|\| [symbol] | −2.071 | −0.358 | −2.158 | −5.218 | −4.77 | −2.498 | −4.815 | 0.013 | 2.455 | −3.577 | 1.407 | 2.285 | −5.273 |
| BCL2L14 | 685643 \|\| [symbol] | 1.2645 | 0.5255 | 0.3455 | 0.9805 | −1.2415 | −1.0805 | −1.1445 | −0.8245 | −1.0065 | −0.8595 | −0.8725 | 0.0355 | −1.4876 |
| BCMP11 | 889987 \|\| [symbol] | 1.153 | 0.815 | −0.584 | −0.514 | −2.108 | −2.227 | −0.793 | −1.418 | −0.946 | −1.312 | −0.385 | −0.037 | −1.781 |
| BEX1 | 881657 \|\| [symbol] | 1.5445 | −0.4645 | 0.1205 | −0.7985 | −1.9045 | −3.5695 | −0.4435 | −0.7025 | 2.4265 | −3.2015 | 0.1085 | 1.8695 | 2.0805 |
| BEX2 | 887414 \|\| [symbol] | 0.9675 | 0.1335 | −0.2635 | −0.1875 | 0.4835 | 0.6785 | 0.4315 | 1.0585 | 0.7355 | 1.4335 | 0.3926 | 0.7445 | −0.1655 |
| BF | 886993 \|\| [symbol] | 0.628 | 0.191 | −0.305 | 0.942 | 1.431 | 1.167 | 0.354 | 0.104 | −0.031 | 1.153 | −0.327 | 0.225 | 0.352 |
| BFSP1 | 681068 \|\| [symbol] | −0.802 | 0.408 | −0.528 | −0.812 | −0.696 | 0.015 | −0.942 | −0.015 | 0.233 | −0.247 | −0.501 | −0.814 | 0.205 |
| BIK | 888391 \|\| [symbol] | −0.903 | −0.818 | −0.033 | 0.298 | −0.027 | 1.212 | 1.107 | −0.06 | −0.669 | 0.699 | 1.195 | 0.46 | 1.767 |
| BIRC2 | 685336 \|\| [symbol] | −0.3815 | −0.3055 | 0.1845 | 0.6485 | 0.2365 | 0.0455 | −0.1005 | 0.0075 | 0.2825 | 0.3315 | 0.5325 | 0.2585 | −0.2855 |
| BIRC5 | 687015 \|\| [symbol] | 0.5825 | 0.5345 | −0.9255 | −1.1575 | 0.5645 | −0.0985 | −0.6705 | 0.2585 | −0.1225 | 0.4795 | −0.6795 | −1.4585 | −0.3455 |
| Bit1 | 885275 \|\| [symbol] | −0.2095 | 0.5585 | −0.2875 | 0.7965 | −0.4965 | 0.1685 | 0.7255 | −0.2185 | 0.2755 | 0.3805 | −0.0145 | 0.5065 | 0.4025 |
| BIVM | 891115 \|\| [symbol] | −0.017 | −0.131 | 0.47 | −0.596 | −0.583 | 0.442 | 0.293 | −0.366 | −0.03 | 1.043 | 0.488 | 0.201 | 0.145 |
| BLVRA | 695027 \|\| [symbol] | −0.092 | 2.438 | −0.88 | 0.85 | −0.773 | 3.058 | 1.226 | −0.046 | −0.122 | 2.628 | 2.203 | 1.556 | −1.913 |
| BM039 | 692354 \|\| [symbol] | 1.537 | −0.405 | −0.693 | −0.314 | −0.121 | −0.107 | 0.027 | −0.709 | −0.68 | −0.368 | −0.123 | −0.588 | 0.173 |
| BMP7 | 697100 \|\| [symbol] | −0.846 | −0.404 | −0.774 | −1.014 | −0.956 | −0.431 | 0.406 | −0.946 | −0.16 | 1.731 | −0.785 | −0.884 | −0.162 |
| BOC | 894025 \|\| [symbol] | −0.0585 | −0.5495 | −1.2075 | 0.0975 | 0.1505 | 0.4915 | 0.2065 | −0.4045 | 0.0875 | 0.4255 | −0.1885 | 0.4805 | −0.7565 |
| BRCA1 | 691229 \|\| [symbol] | −0.3655 | 0.0825 | 0.3265 | −1.0515 | −0.5145 | −0.0915 | 1.1215 | 0.2325 | −0.1095 | −0.4755 | 0.1305 | −0.4005 | −0.0605 |
| BRF2 | 697749 \|\| [symbol] | 0.871 | −0.737 | 0.048 | 1.268 | 0.471 | 0.593 | 0.294 | −0.537 | 0.257 | 0.99 | 0.893 | 1.267 | 2.325 |
| BRI3 | 694633 \|\| [symbol] | 0.555 | −0.087 | 1.11 | 0.73 | −0.512 | −0.375 | 0.089 | −1.21 | −0.664 | 0.091 | −0.727 | 0.544 | 0.55 |
| BRI3BP | 685224 \|\| [symbol] | −0.624 | −0.201 | −0.21 | 0.868 | −1.232 | −0.897 | −1.448 | 0.031 | 0.099 | 0.011 | −0.593 | −0.89 | −0.787 |
| BSPRY | 669357 \|\| [symbol] | 0.0416 | 1.5225 | 0.1285 | −0.3165 | −1.6545 | −0.7595 | 0.8815 | 0.6015 | 0.0755 | 0.9035 | −0.3875 | −0.0785 | −0.6465 |
| BTF3 | 688146 \|\| [symbol] | −0.6285 | 0.3355 | 0.3045 | −1.3585 | −0.8285 | −0.2745 | −0.2685 | 0.6445 | 0.0345 | −0.7295 | −0.0015 | 0.7095 | 0.4355 |
| BTG3 | 881935 \|\| [symbol] | −0.365 | 0.327 | −0.042 | −0.439 | 0.033 | 0.873 | 1.175 | 0.15 | 0.398 | 0.03 | 0.891 | 0.15 | 0.37 |
| BγN2A2 | 685872 \|\| [symbol] | −0.274 | 0.085 | −0.411 | −0.313 | 0.804 | −0.002 | −0.182 | 0.082 | 0.208 | −0.461 | 0.788 | 0.544 | 0.224 |
| BUB1 | 682711 \|\| [symbol] | 0.2205 | −0.3895 | −0.1835 | −0.5245 | −0.0025 | 0.1425 | 0.0315 | −0.5285 | 0.4765 | 0.8745 | 1.3815 | 1.1065 | 2.3185 |
| BUB3 | 683752 \|\| [symbol] | 0.604 | 0.309 | 0.609 | 0.826 | 0.684 | 0.528 | 0.303 | 0.065 | −0.693 | 1.617 | 0.648 | 0.07 | 0.853 |
| BXDC1 | 686690 \|\| [symbol] | −0.48 | 0.59 | 0.16 | −0.093 | 0.213 | −0.244 | −0.233 | 0.268 | 0.191 | −0.395 | 0.585 | 0.279 | 0.22 |
| BZRP | 682336 \|\| [symbol] | 0.983 | −1.723 | −0.717 | 0.474 | −0.426 | −0.968 | −1.701 | −1.09 | −1.238 | −2.113 | −1.293 | −0.239 | −0.988 |
| BZW1 | 689146 \|\| [symbol] | −0.0335 | 1.2015 | 0.1285 | −0.2885 | −0.1555 | −0.1645 | 0.0825 | 0.1165 | 0.0985 | 0.3505 | −0.0175 | −0.6135 | −0.5295 |
| C10orf116 | 694348 \|\| [symbol] | 0.2745 | −0.3065 | −0.0885 | 0.4545 | −0.1075 | 0.0645 | −0.1285 | 0.0755 | 0.6555 | −0.5475 | −0.4425 | −0.1105 | 0.0485 |
| C10orf38 | 696429 \|\| [symbol] | 0.618 | 0.804 | −0.868 | −1.245 | 0.507 | 0.17 | 0.782 | 0.197 | −0.339 | 0.525 | −0.024 | −1.003 | 0.445 |
| C10orf4 | 682455 \|\| [symbol] | 0.2345 | 0.1745 | −0.0105 | −0.0715 | 0.0695 | 0.2345 | 0.2445 | 0.1905 | 0.0995 | 0.8965 | 0.0815 | −0.0615 | 0.8695 |
| C10orf45 | 895197 \|\| [symbol] | −0.7515 | −0.5105 | 1.1385 | −0.3445 | −0.3145 | −0.4815 | −0.4825 | −0.2045 | 1.0775 | −0.5135 | 0.0455 | 0.6455 | −0.3565 |
| C10orf7 | 088194 \|\| [symbol] | −0.0655 | 0.4125 | −0.3405 | −0.2385 | 0.3215 | 0.1765 | −0.1655 | 1.0595 | 1.2485 | −0.0945 | 0.3625 | 0.5985 | 0.3765 |
| C10orf82 | 693231 \|\| [symbol] | 0.287 | 0.126 | 0.34 | 0.097 | 0.48 | 0.912 | −0.474 | −0.324 | 0.509 | 0.52 | 0.401 | −0.399 | 0.258 |
| C11orf24 | 894704 \|\| [symbol] | −0.0155 | −0.5055 | −0.9155 | 0.2975 | −1.5045 | −0.7875 | −0.9455 | 0.0455 | −0.3345 | −0.8185 | 0.1375 | 0.5695 | 0.6025 |
| C12orf14 | 892853 \|\| [symbol] | −0.123 | 0.862 | −0.531 | 0.048 | 0.434 | 0.772 | 0.359 | −0.017 | 0.13 | 0.963 | 0.373 | −0.197 | 0.976 |
| C13orf21 | 697705 \|\| [symbol] | 0.031 | −0.058 | 0.042 | 0.729 | −0.548 | −0.567 | −0.261 | −0.057 | 0.089 | 0.042 | 0.325 | 0.389 | 0.117 |
| C13orf23 | 691314 \|\| [symbol] | 0.14 | 0.25 | −0.353 | 0.498 | 0.55 | 0.525 | 1.001 | −0.185 | 0.586 | 0.647 | 0.091 | 0.382 | 0.823 |
| C14orf94 | 894118 \|\| [symbol] | 1.24 | −0.015 | −0.606 | 0.476 | 1.85 | −0.32 | 1.18 | 0.01 | −0.3 | −1.25 | −0.24 | 0.08 | −0.95 |
| C15orf23 | 681265 \|\| [symbol] | 2.7625 | 2.4115 | 3.2855 | 1.9045 | 2.2195 | 3.0525 | 0.1345 | 3.0745 | 3.2415 | 1.6805 | 1.4315 | 1.0895 | 2.1045 |
| C16orf45 | 772706 \|\| [symbol] | −1.154 | 0.036 | −0.39 | −1.131 | 0.055 | −0.547 | 0.095 | 1.327 | −0.369 | −1.605 | −1.012 | −1.779 | −0.454 |
| C17orf37 | 686313 \|\| [symbol] | 0.057 | −0.098 | −0.393 | −0.75 | 0.261 | −0.04 | 0.772 | 0.492 | 0.735 | 0.29 | 0.186 | 0.274 | −0.027 |
| C18orf1 | 584420 \|\| [symbol] | 0.996 | −0.157 | 0.596 | 1.401 | 1.409 | 0.568 | 2.104 | −0.616 | −1.054 | 2.063 | 0.044 | 0.643 | 0.137 |
| C19orf10 | 687290 \|\| [symbol] | 0.1065 | −0.6025 | 1.0405 | −0.8055 | −0.9125 | −0.4695 | −0.4965 | 0.2205 | 0.2055 | −0.4515 | −0.7985 | −1.2595 | −0.1535 |
| C19orf32 | 697502 \|\| [symbol] | 0.2585 | −1.0655 | −0.9795 | 0.9875 | −1.2715 | −0.4515 | −0.5085 | −1.2115 | −2.2845 | −0.3505 | −1.0205 | −0.0205 | −2.0645 |
| C19orf33 | 693018 \|\| [symbol] | −0.15 | −0.333 | −0.24 | 0.059 | −2.564 | −1.674 | 1.715 | −1.8 | −0.682 | −0.145 | 1.245 | 0.7 | −1.536 |
| C1orf24 | 689493 \|\| [symbol] | 0.182 | 1.01 | −1.229 | 1.172 | 0.388 | 0.298 | 0.545 | 0.867 | 1.136 | −0.797 | −0.653 | 0.333 | −0.754 |
| C1orf34 | 694656 \|\| [symbol] | 0.0215 | −0.4085 | 0.0155 | 0.8105 | −0.2275 | 0.5205 | 1.1225 | 0.3705 | 0.3285 | −0.8245 | −0.0165 | 0.3735 | −0.2345 |
| C20orf103 | 695553 \|\| [symbol] | 0.7795 | −1.4825 | −1.0885 | −0.8345 | −0.0005 | 0.1305 | 0.7455 | −0.6765 | 1.4135 | 0.3665 | −0.2215 | −0.3655 | −1.8385 |
| C20orf149 | 691505 \|\| [symbol] | 0.853 | −0.494 | 0.134 | −0.043 | 1.542 | 0.978 | 1.578 | 1.125 | 0.359 | 0.769 | −0.017 | −0.567 | 1.411 |
| C20orf23 | 887940 \|\| [symbol] | −0.589 | −0.992 | −0.657 | −0.484 | −1.359 | −1.431 | −1.188 | −1.219 | −1.073 | −1.119 | −0.625 | −0.373 | −0.805 |
| C20orf3 | 685458 \|\| [symbol] | 0.054 | −0.37 | −0.202 | −0.132 | 0.339 | 0.004 | −0.227 | −0.407 | −0.367 | 0.276 | 0.045 | −0.015 | −0.286 |
| C20orf35 | 693279 \|\| [symbol] | −0.228 | −0.685 | 0.055 | −0.893 | 0.071 | 0.621 | −0.352 | 0.122 | −0.011 | −0.144 | 0.198 | −0.153 | 0.468 |
| C20orf43 | 687406 \|\| [symbol] | 0.1735 | −0.1855 | −0.3215 | 0.6825 | 0.5005 | 0.3995 | 0.9845 | −0.1195 | 0.2005 | 0.4965 | 0.1465 | 0.3495 | 0.3845 |
| C20orf8 | 838651 \|\| [symbol] | −0.287 | −0.39 | 0.254 | 0.681 | −0.298 | 0.109 | −0.088 | −0.202 | 0.187 | 1.39 | 0.16 | 0.688 | −0.167 |
| C21orf45 | 681352 \|\| [symbol] | −0.895 | 0.146 | −0.087 | 0.24 | −0.769 | −0.038 | −0.65 | −0.272 | 0.077 | 0.18 | 0.809 | 0.646 | −0.328 |
| C22orf18 | 881943 \|\| [symbol] | −0.429 | 0.044 | 0.279 | −0.034 | −0.026 | −0.747 | −0.148 | −0.713 | −0.227 | −0.413 | −0.002 | 0.248 | −0.314 |
| C2F | 687401 \|\| [symbol] | 1.533 | 0.681 | −0.308 | 1.474 | −0.451 | −1.385 | 0.269 | −0.868 | −0.325 | −1.057 | 0.718 | 1.419 | 2.242 |
| C2orf17 | 093373 \|\| [symbol] | −1.382 | 0.959 | −0.091 | 0.064 | −0.404 | 1.022 | −0.042 | 1.746 | 1.189 | −0.235 | 0.328 | 0.13 | 0.184 |
| C4A | 589714 \|\| [symbol] | 0.138 | −0.071 | −0.081 | −0.143 | −1.171 | −0.102 | 0.08 | −0.181 | 0.103 | 0.929 | −0.087 | 0.769 | −0.887 |
| C5orf13 | 694921 \|\| [symbol] | 0.183 | −0.193 | −0.398 | −0.16 | −0.173 | 0.018 | −0.009 | −0.308 | −0.071 | −0.308 | 0.021 | −0.18 | −0.45 |
| C6orf108 | 894988 \|\| [symbol] | 0.672 | 0.185 | −0.806 | −0.584 | −0.034 | −0.318 | −1.549 | −0.392 | 0.232 | 0.071 | 0.444 | 0.431 | −1.595 |
| C6orf166 | 694215 \|\| [symbol] | 0.426 | −0.826 | −0.465 | −1.185 | −0.193 | −0.516 | −0.778 | −0.89 | 0.436 | −0.466 | −0.084 | 0.673 | −1.441 |
| C6orf192 | 801969 \|\| [symbol] | 0.0585 | −0.4985 | 0.0765 | 0.5635 | 0.0785 | −0.0565 | −0.0725 | −0.1965 | −0.2035 | 0.3825 | 0.0185 | 0.2845 | 0.3795 |
| C6orf211 | 692990 \|\| [symbol] | 1.42 | 1.704 | 0.754 | 1.873 | 1.675 | 2.109 | 1.779 | 1.038 | 0.593 | 0.118 | 0.123 | −0.008 | 1.681 |
| C6orf49 | 692808 \|\| [symbol] | 0.0945 | 0.6835 | −0.3765 | 1.0565 | 0.5415 | 1.3895 | 1.4395 | 1.3425 | 0.2375 | 0.8195 | 1.6045 | 1.8655 | 1.5315 |
| C6orf85 | 693055 \|\| [symbol] | 0.3765 | −0.8045 | −0.2635 | −0.9055 | −0.0185 | −0.1435 | −0.7395 | −0.3395 | 0.3845 | 0.1595 | −0.6425 | −0.9335 | −0.3485 |
| C7orf24 | 585020 \|\| [symbol] | −0.53 | −0.955 | −0.433 | −1.023 | −2.253 | −1.173 | −0.811 | −1.167 | −0.396 | −1.437 | −0.596 | −1.514 | −1.035 |
| C9orf10 | 982781 \|\| [symbol] | 1.365 | −0.831 | −0.372 | 1.942 | 2.84 | 1.542 | 1.766 | −1.027 | −0.759 | 0.041 | −0.027 | −1.595 | 1.471 |
| C9orf121 | 692685 \|\| [symbol] | 0.819 | −0.29 | 0.34 | 0.792 | 1.040 | 0.267 | 0.651 | 0.018 | 0.647 | −0.342 | 0.183 | 0.442 | 0.758 |
| C9orf58 | 686494 \|\| [symbol] | 1.3005 | 0.0785 | −0.0175 | 0.7095 | −0.3495 | −0.4165 | −0.1985 | −0.1595 | −0.4015 | 0.2155 | 0.3245 | 0.2975 | −0.7565 |
| C9orf88 | 685307 \|\| [symbol] | −1.75 | −2.57 | 1.137 | −0.865 | −1.463 | −1.109 | −1.69 | −1.177 | 0.531 | −2.375 | 0.377 | 0.714 | 0.944 |
| C9orf9 | 696998 \|\| [symbol] | 0.258 | −0.9 | 0.008 | 1.132 | 0.093 | 0.108 | −0.756 | −0.314 | −0.592 | 0.388 | 2.001 | 1.632 | −0.105 |
| CA12 | 697341 \|\| [symbol] | 0.427 | −1.141 | −0.732 | 1.265 | −0.005 | 0.178 | −0.708 | −0.327 | −0.699 | 0.532 | 1.867 | 1.038 | 0.066 |
| CA8 | 681755 \|\| [symbol] | 0.715 | 0.235 | −0.551 | 0.297 | −0.029 | 0.077 | −0.374 | −0.253 | −0.096 | −0.209 | −0.227 | −0.134 | −0.38 |
| CA8 | 683844 \|\| [symbol] | −0.1125 | 0.3715 | 0.2765 | 0.1405 | −0.1835 | 0.1005 | 0.0745 | −0.0655 | −0.0775 | 0.4525 | −0.2295 | −0.1195 | −0.0325 |
| CACNA1G | 687482 \|\| [symbol] | 0.387 | −0.246 | 1.214 | −0.509 | 0.197 | −0.055 | −0.702 | 0.178 | 0.266 | 0.146 | −0.032 | −0.272 | −0.000 |
| CALCA | 866738 \|\| [symbol] | 3.871 | 2.462 | −0.282 | 2.788 | 2.447 | 0.878 | 2.677 | −0.809 | −0.62 | 3.713 | 2.297 | 0.782 | 3.207 |
| CALML4 | 685502 \|\| [symbol] | 0.2085 | −0.5895 | 0.7675 | −1.8235 | −0.1315 | 0.5865 | 0.2005 | 0.4875 | −0.2925 | −1.8155 | 0.0315 | −0.2495 | 1.0825 |
| CALML5 | 866121 \|\| [symbol] | −0.5535 | −0.4685 | −0.1765 | −0.3345 | 0.1155 | −1.1455 | −0.4575 | −0.2035 | 0.4265 | −0.6605 | −0.3105 | 0.8985 | 0.5615 |
| CaMKIINa [symbol] | 891045 \|\| [symbol] | 0.126 | −0.199 | −0.711 | −0.539 | 0.005 | 0.311 | 0.341 | −0.266 | 0.089 | −0.588 | −0.153 | −0.34 | −0.09 |
| CAP2 | 693946 \|\| [symbol] | 3.134 | 2.973 | −0.147 | 2.402 | 2.164 | 1.06 | 2.149 | −0.555 | −0.751 | 2.018 | 1.135 | 1.076 | 1.29 |
| CAP350 | 695521 \|\| [symbol] | 0.5325 | 0.2225 | −0.2365 | −0.2565 | −0.1975 | −0.9115 | −0.3355 | −0.4905 | −0.3745 | −0.9445 | 0.0855 | −0.7115 | −0.3015 |
| CAPN13 | 691887 \|\| [symbol] | 0.722 | 0.088 | −0.574 | 0.555 | 1.571 | 1.075 | 0.82 | 0.025 | −0.967 | 0.459 | 0.004 | 0.53 | 2.818 |
| CAPN9 | 697168 \|\| [symbol] | −0.0965 | −0.3055 | −0.2465 | 0.3985 | −0.3825 | −0.0335 | 0.1455 | −0.2945 | −0.1715 | 0.0765 | −0.3555 | −0.1455 | −0.0875 |
| CARD14 | 681056 \|\| [symbol] | −0.2555 | 0.8566 | 0.4995 | −0.6515 | −1.4495 | −0.6956 | −1.2705 | 0.3305 | −0.4015 | −0.1225 | 0.8775 | 1.0945 | −1.1575 |
| CART | 697059 \|\| [symbol] | 0.0735 | −0.0875 | 0.0855 | −0.4685 | 0.5065 | 0.9585 | 0.4525 | 0.2215 | 0.1625 | −0.9545 | −0.1625 | −0.4565 | 0.4595 |
| CASP1 | 682686 \|\| [symbol] | −0.232 | −0.08 | 0.306 | −1.294 | −0.408 | 0.069 | 0.331 | 0.157 | 0.232 | −0.782 | 0.278 | 0.792 | 0.083 |
| CASP3 | 998605 \|\| [symbol] | 0.798 | 0.089 | 0.711 | 0.173 | −0.113 | −0.012 | 0.509 | 0.54 | 0.028 | 0.392 | −0.13 | 0 | 0.194 |
| CASP7 | 693491 \|\| [symbol] | −0.0155 | −0.1005 | −0.8775 | −1.2095 | −0.3555 | −0.9955 | 1.1245 | 0.0545 | −0.3955 | −0.6155 | 0.3145 | 0.3605 | −0.6455 |
| C9LN1 | 885274 \|\| [symbol] | −0.099 | −0.175 | 0.523 | 1.324 | −1.004 | −0.456 | 0.577 | 0.115 | 0.624 | 1.104 | 0.971 | 1.079 | 2.137 |
| CBR1 | 784387 \|\| [symbol] | −0.3425 | 0.6785 | 0.0815 | 1.6155 | 0.6495 | 1.4775 | −0.7035 | −0.1495 | −0.4615 | 1.3975 | 1.0655 | 0.0355 | 0.9455 |
| CBR3 | 683488 \|\| [symbol] | 0.032 | −0.072 | 0.164 | 0.549 | −0.17 | −0.017 | −0.27 | 0.124 | 0.066 | −0.181 | −0.079 | 0.346 | 0.2 |
| CBR4 | 697391 \|\| [symbol] | 1.8895 | −0.3265 | −2.1065 | −1.1565 | −0.3865 | −1.0655 | 1.0425 | −0.9105 | −2.1275 | 0.6045 | −0.4085 | −1.4715 | 1.9425 |
| CCL24 | 681864 \|\| [symbol] | −0.633 | 0.098 | 0.28 | −0.568 | −0.249 | 0.059 | 0.485 | −0.182 | 0.225 | 0.557 | 1.197 | −0.136 | 0.864 |
| CCL29 | 885513 \|\| [symbol] | −0.235 | −0.501 | 0.311 | −1.816 | −0.732 | −0.352 | −2.304 | −1.14 | 0.231 | −0.533 | 0.503 | 0.398 | −1.61 |
| CCNB2 | 694924 \|\| [symbol] | 0.4495 | 0.2225 | 0.0605 | 0.3415 | −0.0805 | 0.8405 | 0.1515 | 0.1995 | 0.7795 | 0.5255 | 1.2875 | 0.7365 | 0.5295 |
| CCND1 | 689117 \|\| [symbol] | −1.144 | −0.474 | −0.791 | −0.583 | 0.543 | 1.297 | −1.084 | −0.216 | −0.489 | −1.916 | −0.287 | −1.312 | −0.179 |
| CCNE1 | 685647 \|\| [symbol] | −0.2265 | 0.2205 | 0.1655 | 0.4825 | −0.3925 | −0.7775 | −1.1825 | 0.3305 | 0.2945 | −0.4395 | 0.0375 | 0.1595 | −0.0515 |
| CCNG2 | 686672 \|\| [symbol] | −0.248 | −0.176 | 0.33 | −0.382 | 0.554 | −0.357 | 0.65 | −0.129 | −0.479 | 0.856 | 0.272 | 0.023 | 0.229 |
| CCNH | 888140 \|\| [symbol] | −0.1215 | 0.1865 | −0.6375 | −0.2135 | −0.5155 | 0.2285 | −0.3885 | 0.2475 | −0.8235 | 0.5245 | 0.7055 | 0.9255 | −0.4785 |
| CD38 | 891273 \|\| [symbol] | −0.1005 | −0.1935 | 0.3735 | 0.6435 | −0.1075 | −0.1735 | −0.1025 | −0.1225 | −0.2195 | −0.1785 | −0.3115 | 0.7975 | 0.0476 |
| C030 | 696702 \|\| [symbol] | −0.6935 | 0.0546 | −0.0915 | −0.5115 | 0.1545 | −0.5235 | 0.0565 | −0.2035 | 0.3455 | −0.0145 | 0.3285 | −0.2945 | −0.6535 |
| CD4 | 691128 \|\| [symbol] | 0.3495 | 0.1885 | 0.3845 | −0.3595 | −0.0985 | −1.0355 | −1.1165 | −0.1405 | −0.4875 | −0.2705 | −0.6125 | −1.0145 | −1.1775 |
| CD58 | 695421 \|\| [symbol] | −0.273 | −0.188 | 0.252 | 0.005 | −0.296 | 0.13 | 0.898 | −0.51 | 0.296 | 1.047 | 0.948 | 0.541 | 0.696 |
| CD50 | 682450 \|\| [symbol] | −0.2165 | 1.0205 | 1.5625 | 0.1635 | −0.5285 | 3.1745 | 0.4445 | −0.9575 | −0.4115 | 0.8245 | 2.4035 | 1.8155 | 4.8535 |
| CDC2 | 694849 \|\| [symbol] | −0.5315 | 0.1645 | −0.2055 | −0.9135 | −0.6545 | 0.3845 | 0.5745 | 0.0615 | 0.6705 | −0.2835 | 0.6645 | 0.2805 | −0.1375 |
| CDC6 | 693742 \|\| [symbol] | −1.1605 | −0.7325 | 0.6905 | 0.0925 | −0.2705 | 0.6295 | 0.2785 | −0.2455 | 0.3345 | 0.6335 | 1.4045 | 0.8185 | 1.7345 |
| CDCA1 | 693999 \|\| [symbol] | −0.7 | 1.176 | 0.11 | −0.378 | −0.451 | 0.847 | −0.41 | 0.332 | −0.205 | −0.623 | 0.253 | −0.135 | 0.659 |
| CDCA5 | 896784 \|\| [symbol] | 0.758 | 0.681 | 0.454 | 0.211 | 1.273 | 1.236 | 0.703 | 0.87 | 1.056 | 0.143 | 0.193 | −0.477 | 1.05 |
| CDCA7 | 686991 \|\| [symbol] | 1.4155 | 1.8745 | −0.5475 | −0.7295 | −1.0065 | −0.8805 | −0.4085 | −0.3315 | −0.6915 | −0.5645 | 1.0435 | −0.3705 | 1.0205 |
| CDH1 | 693949 \|\| [symbol] | −0.388 | −0.553 | −0.2 | 0.321 | 0.283 | 0.684 | 0.49 | −0.306 | 0.118 | −0.293 | −0.099 | 0.1 | 0.69 |
| CDH3 | 689567 \|\| [symbol] | −0.4545 | 0.4025 | 1.2815 | 1.4645 | 0.2375 | 0.3805 | −0.1785 | −0.0085 | 0.1065 | 0.7235 | 1.0855 | 0.0765 | 0.6785 |
| CDK2 | 690323 \|\| [symbol] | −0.0605 | −0.3135 | 0.4175 | −0.0895 | −0.3325 | 0.4395 | 0.9015 | −0.0285 | −0.4895 | −0.0585 | −0.1845 | 0.3065 | 0.7205 |
| CDKN2A | 692881 \|\| [symbol] | 0.5045 | 0.8065 | −0.1365 | −0.3415 | 0.2365 | 0.3405 | 0.7235 | 0.1145 | 0.0125 | 0.9335 | 0.5215 | 0.7825 | 0.5016 |
| CDKN2D | 682687 \|\| [symbol] | 0.04 | 0.865 | −0.507 | 0.169 | −0.406 | 0.862 | −0.153 | −0.222 | 0.011 | −0.043 | 0.307 | 0.945 | 0.549 |
| CDKN3 | 684517 \|\| [symbol] | 0.176 | −0.018 | 3.458 | 0.088 | −0.451 | 0.19 | 0.11 | 0.51 | 0.485 | 0.08 | −0.375 | −0.055 | −0.316 |
| CDT1 | 686706 \|\| [symbol] | −0.5925 | −0.2885 | −0.8205 | −0.8815 | −0.0435 | −1.5655 | −1.5805 | 0.3495 | 0.1025 | −1.0635 | −0.4555 | −0.2045 | 0.3616 |
| CE9PG | 688002 \|\| [symbol] | 0.012 | 0.001 | −0.189 | −0.162 | 0.189 | 1.273 | 0.875 | 0.23 | −0.255 | 0.296 | 0.281 | −0.298 | −0.001 |
| CELSR1 | 686084 \|\| [symbol] | −0.275 | −0.326 | 0.334 | −0.336 | −0.317 | 0.809 | −0.007 | 0.34 | 0.3 | 0.342 | 0.569 | −0.038 | −0.091 |
| CENPA | 897624 \|\| [symbol] | −0.558 | −0.618 | 0.504 | −0.897 | −0.496 | 1.007 | 1.17 | −0.199 | 0.268 | −0.96 | 1.494 | −0.235 | 0.291 |
| CENPE | 683395 \|\| [symbol] | 0.189 | 0.018 | 0.042 | 0.143 | 0.203 | 0.165 | 0.218 | −0.436 | 0.434 | −0.521 | −0.128 | 0.227 | 0.358 |
| CENPF | 691312 \|\| [symbol] | 0.8725 | −0.0916 | 0.4715 | −0.2785 | −0.1616 | 0.1575 | −0.0465 | −0.0305 | −0.1648 | −0.1585 | −0.6985 | 0.0015 | −0.4295 |
| CENTG3 | 687681 \|\| [symbol] | −0.5605 | −0.1175 | −0.2755 | 0.3435 | −1.0535 | −0.3215 | −1.0985 | −0.2635 | 0.2435 | 0.3255 | −0.0935 | 0.0485 | −0.5905 |
| CES2 | 897031 \|\| [symbol] | −0.567 | −0.047 | 1.581 | −0.523 | −1.025 | −0.651 | −0.237 | 0.317 | −0.049 | −0.885 | −0.31 | −0.098 | 0.494 |
| CETN3 | 692233 \|\| [symbol] | −0.8295 | 0.8705 | −0.4275 | −0.7475 | −0.4135 | 0.0395 | 0.0575 | 0.0795 | −0.7826 | 1.4795 | 0.9975 | 0.6535 | 0.7625 |
| CGA | 893821 \|\| [symbol] | −0.5765 | −0.1205 | −0.9595 | −0.7145 | −0.7415 | −0.5225 | −0.1505 | −0.1595 | −0.5845 | −0.0155 | 1.2595 | 1.4525 | −0.3575 |
| CGI-12 | 683023 \|\| [symbol] | −1.0805 | −0.7155 | −0.7945 | −1.9195 | −0.4965 | −0.7065 | 1.3625 | −1.3055 | 0.4785 | −2.0495 | 2.7955 | 1.2055 | 0.2055 |
| CGI-49 | 693157 \|\| [symbol] | −0.011 | −0.467 | −0.598 | −2.814 | −1.313 | 0.844 | 0.241 | −1.056 | −0.259 | −1.507 | −1.216 | −0.979 | −1.511 |
| CGI-4#z899; | 684734 \|\| [symbol] | 0.127 | −0.956 | −1.754 | 0.498 | −0.056 | −0.291 | −0.013 | −0.043 | 0.269 | −0.491 | −1.526 | −0.78 | −0.545 |
| CGN | 688407 \|\| [symbol] | −0.797 | 0.023 | 0.053 | 0.433 | −0.883 | 0.009 | 1.757 | −1.084 | −0.28 | 0.007 | 0.42 | 1.103 | 1.067 |
| CGNL1 | 890560 \|\| [symbol] | 0.4595 | 1.3725 | 0.0535 | 1.9195 | −1.4505 | 0.2355 | 1.5355 | −0.2945 | −0.1565 | 1.1615 | 0.7945 | 0.3785 | 1.8875 |
| CHAF1B | 882830 \|\| [symbol] | 0.015 | 0.536 | −0.018 | 0.407 | 0.078 | −0.105 | 1.90 | −0.507 | −0.287 | 0.859 | 0.908 | 0.709 | 0.207 |
| CHC1 | 682220 \|\| [symbol] | −0.13 | −0.758 | −0.072 | 0.843 | 0.156 | 0.424 | 0.286 | −0.357 | 0.134 | −0.043 | 0.147 | 0.394 | −0.132 |
| CHCHD2 | 681919 \|\| [symbol] | 2.046 | 0.35 | 0.607 | 0.917 | −0.728 | −1.15 | −0.515 | −0.401 | −1.28 | −0.073 | 0.609 | 0.001 | −0.831 |
| CHCH05 | 886549 \|\| [symbol] | 0.965 | −1.242 | −0.8 | 1.03 | 1.455 | −1.85 | −1.335 | −1.583 | −0.67 | −0.273 | −0.793 | 2.245 | −0.958 |
| CHI3L1 | 889913 \|\| [symbol] | −0.358 | 0.12 | −0.037 | 0.709 | −0.384 | 0.073 | 0.379 | 0.192 | 0.08 | 0.557 | −0.042 | −0.232 | 0.308 |
| CHI3L2 | 883767 \|\| [symbol] | −0.046 | 0.127 | 0.517 | 0.306 | −0.514 | −0.028 | −0.333 | 0.639 | −0.08 | 0.25 | 0.068 | 0.29 | −0.202 |
| CHKA | 889809 \|\| [symbol] | 0.168 | 0.026 | −0.404 | −0.244 | −0.18 | −0.246 | −0.286 | −0.141 | −0.804 | −0.059 | 0.159 | −0.092 | −0.343 |
| CHCDL | 594645 \|\| [symbol] | −0.0965 | 0.0215 | 0.5575 | 0.1085 | 0.0685 | −0.1015 | −0.3335 | 0.3295 | −0.0295 | −0.4055 | −0.2255 | 0.0885 | −0.2195 |
| CHRDL2 | 686385 \|\| [symbol] | 0.128 | −0.007 | −0.479 | 0.11 | 2.673 | −0.534 | −0.467 | 0.07 | 0.398 | 0.205 | −0.612 | −0.463 | −0.819 |
| CHST2 | 691695 \|\| [symbol] | −0.5235 | −0.4145 | −0.0705 | 0.1125 | −0.0385 | 0.0815 | −0.5245 | −0.0735 | 0.0845 | −0.5255 | 0.1015 | 0.2535 | 0.2115 |
| CHURC1 | 684314 \|\| [symbol] | −0.079 | −0.047 | 0.097 | 0.068 | −0.322 | −0.131 | −0.658 | 0.248 | −0.478 | −0.472 | −0.571 | −0.656 | −1.168 |
| CIR | 695941 \|\| [symbol] | 0.7195 | 0.4865 | −0.2215 | −0.1875 | −0.1865 | 0.4075 | −0.0205 | 0.2135 | 0.0655 | 0.4515 | −0.1315 | 0.0875 | −0.7095 |
| CIRBP | 585402 \|\| [symbol] | −0.6645 | 0.2975 | −0.6285 | −0.2975 | −0.0745 | 1.5015 | 0.6945 | 0.0405 | −0.4225 | −1.0735 | −0.9735 | −0.8355 | −1.2675 |
| CKLF | 688221 \|\| [symbol] | −0.0975 | −0.7155 | −0.8565 | −1.4195 | −1.8315 | 0.1895 | −0.7525 | −0.7815 | −0.7185 | −0.9855 | −1.2265 | −1.0235 | −1.2815 |
| CKLFSF6 | 681850 \|\| [symbol] | 1.3035 | −1.0225 | 1.1645 | 0.9485 | −1.7255 | −1.3985 | 0.1685 | −1.1435 | 1.4975 | 2.1175 | 0.9215 | 0.5465 | 2.0635 |
| CKLFSF7 | 694344 \|\| [symbol] | −0.077 | −0.037 | 0.063 | −0.614 | −0.472 | −0.133 | 1.32 | −0.235 | −0.239 | 0.028 | 0.73 | 0.473 | 0.513 |
| CKMT1 | 692148 \|\| [symbol] | −0.677 | −0.558 | −0.337 | −1.021 | −0.529 | −0.599 | 0.435 | −0.668 | 0.14 | 0.692 | 0.182 | −0.454 | 0.018 |
| CKS1B | 696332 \|\| [symbol] | −0.187 | 0.388 | −0.092 | 0.613 | 0.354 | 0.48 | 0.139 | 0.616 | −0.086 | 1.165 | 0.223 | 0.259 | −0.516 |
| CKS2 | 681352 \|\| [symbol] | −0.0435 | 0.0555 | −0.6705 | −0.7185 | 0.4855 | 0.5755 | 1.7275 | −0.1725 | −0.6545 | −0.8525 | −0.7725 | −0.6925 | 0.1546 |
| CL640 | 695108 \|\| [symbol] | 1.0735 | 0.5095 | −1.6155 | 0.7055 | 0.2495 | −0.6035 | 1.9485 | −0.4155 | −0.6115 | −0.0855 | 0.2145 | 0.9575 | 1.4335 |
| CLDN23 | 683062 \|\| [symbol] | 0.8115 | 0.7795 | 1.2845 | 0.1165 | −0.2665 | 0.4745 | 1.8105 | 0.0235 | −0.3215 | −1.3505 | −0.4215 | 0.1135 | −0.1765 |
| CLDN3 | 886923 \|\| [symbol] | 2.5335 | 1.3595 | −0.8155 | −0.7345 | 0.2795 | −0.1396 | 2.1315 | −1.3055 | −0.8595 | 2.7505 | −0.3095 | −0.5855 | 0.4095 |
| CLDN4 | 881712 \|\| [symbol] | 1.535 | −0.72 | −1.502 | 2.397 | −0.245 | −0.971 | −0.532 | −0.845 | −2.388 | 0.194 | −0.369 | 1.057 | 4.484 |
| CLDN8 | 595858 \|\| [symbol] | 0.0025 | −0.3435 | −1.3775 | −0.5515 | −0.8655 | −0.8195 | −1.0155 | −0.2925 | −0.3035 | 0.3885 | −0.1855 | −0.0545 | −0.8275 |
| CLGN | 691474 \|\| [symbol] | −0.9005 | 0.2585 | 0.9825 | −0.7445 | −0.7015 | −0.0715 | −0.8505 | −0.1205 | 0.0325 | −0.1425 | 0.4235 | 0.0585 | 0.2485 |
| CLIC6 | 692133 \|\| [symbol] | −0.221 | 0.502 | 0.073 | 0.205 | 0.501 | 0.043 | 0.351 | 0.654 | −0.003 | −0.461 | −0.119 | −0.438 | −1.103 |
| CLNS1A | 893066 \|\| [symbol] | 2.3125 | 0.0355 | −0.0485 | 2.5055 | 3.4035 | −1.9115 | 0.8515 | −1.2235 | −1.7475 | −3.5145 | −0.3345 | 0.3505 | 0.2225 |
| CLTC | 894429 \|\| [symbol] | 0.035 | 1.177 | −0.28 | −0.667 | −0.435 | 0.027 | −0.922 | −0.097 | 0.343 | 0.681 | 0.256 | −0.262 | −0.929 |
| CLU | 881706 \|\| [symbol] | 0.02 | 0.8 | 0.41 | −0.73 | −0.539 | 0.436 | −0.095 | −0.213 | −0.368 | 1.101 | 1.047 | 0.12 | 1.003 |
| CMAS | 682207 \|\| [symbol] | 0.3445 | 1.2805 | −0.0595 | 0.2315 | −0.2675 | −0.2945 | −0.5605 | 0.0145 | 0.2715 | 1.0235 | 0.2375 | −0.0725 | 0.2255 |
| CML65 | 582080 \|\| [symbol] | −0.671 | 0.017 | −0.405 | −0.282 | −0.604 | 1.48 | 0.165 | −0.57 | −0.455 | 0.133 | −0.443 | −0.083 | −0.8 |
| CNIH | 685679 \|\| [symbol] | −0.02 | −0.011 | −0.216 | 0.634 | 0.848 | 0.172 | −0.201 | 0.484 | 0.351 | −0.150 | 1.004 | 0.501 | 0.331 |
| CNOT10 | 686504 \|\| [symbol] | −0.3765 | −0.5875 | −0.1175 | 0.2775 | 0.7385 | 0.3575 | 0.3995 | 0.1725 | −0.1755 | −0.2775 | −0.3935 | −0.6575 | −0.1565 |
| CNOT2 | 688501 \|\| [symbol] | 0.42 | −0.043 | 0.301 | 0.836 | 0.446 | 1.072 | 0.35 | 0.756 | 0.028 | 0.556 | −0.304 | −0.291 | 0.358 |
| CNOT7 | 687730 \|\| [symbol] | 3.441 | 0.478 | 2.882 | 2.987 | 0.208 | 3.282 | 4 | 2.737 | 2.296 | 1.068 | 2.595 | 2.394 | 5.418 |
| CNP | 684883 \|\| [symbol] | −0.3325 | −0.8595 | −0.6025 | −0.2885 | −0.2405 | −0.4685 | −0.8385 | −0.2675 | −0.3205 | 0.3945 | −0.7025 | −0.5755 | 0.0205 |
| CNTNAP2 | 687882 \|\| [symbol] | −0.109 | 0.137 | 0.307 | 0.739 | −0.116 | −0.091 | −0.385 | 0.061 | 0.185 | 0.342 | 0.059 | −0.186 | 0.343 |
| COASY | 594548 \|\| [symbol] | −1.3905 | 0.4285 | −1.6775 | −0.0765 | −2.2585 | −0.8795 | 0.3285 | 0.6095 | −0.9625 | −2.1305 | −0.0005 | −0.5485 | −0.5025 |
| COL11A2 | 696658 \|\| [symbol] | −0.658 | −0.671 | −0.401 | −0.048 | −0.094 | 0.196 | −0.578 | 0.352 | 0.349 | −1.366 | −0.085 | −0.274 | −1.298 |
| COL27A1 | 090790 \|\| [symbol] | −0.393 | −0.227 | 1.204 | 1.548 | 0.524 | 0.041 | −1.195 | −0.411 | −0.062 | −0.203 | 0.225 | 1.777 | 0.086 |
| COL4A5 | 695638 \|\| [symbol] | 0.5015 | −0.9715 | −1.9415 | 1.0515 | 0.2785 | −1.1695 | 1.2815 | −0.0375 | −0.7135 | 1.0325 | 0.1085 | 0.7725 | 0.7455 |
| COL8A1 | 688215 \|\| [symbol] | −0.2 | 0.542 | 0.061 | −0.403 | −0.748 | −0.714 | −0.643 | 0.466 | 0.168 | −0.465 | 0.702 | 0.475 | −0.031 |
| COL8A3 | 694474 \|\| [symbol] | −0.208 | 0.811 | −0.492 | −0.213 | 0.023 | 0.261 | −0.388 | 0.637 | −0.141 | 0.465 | 1.207 | 1.505 | 0.658 |
| COMMD1 [symbol] | 885920 \|\| [symbol] | −0.3545 | 0.1925 | 0.6675 | 1.1275 | 0.3845 | −0.4015 | −0.0685 | 0.3255 | 0.2395 | −0.1755 | 0.2435 | 0.2425 | 0.0445 |
| COMMD5 | 883292 \|\| [symbol] | −0.3275 | −0.2915 | 0.8305 | 0.8505 | −0.5055 | −0.4855 | 0.9875 | 0.7275 | 0.4305 | −0.8275 | 0.0355 | −0.0705 | 0.9835 |
| COPS7B | 888043 \|\| [symbol] | 0.04 | −0.279 | 0.692 | 0.554 | 0.278 | −0.085 | 0.402 | −0.462 | −0.35 | 0.188 | 0.217 | −0.178 | 0.144 |
| CORO6 | 691905 \|\| [symbol] | 0.1185 | −0.3305 | 0.6705 | 0.4255 | −0.2515 | 0.3125 | 0.2705 | −0.2905 | 0.4105 | 1.0615 | 0.0005 | 0.8425 | −0.4495 |
| CORT | 688126 \|\| [symbol] | 0.008 | −0.100 | −0.116 | −0.755 | −0.592 | −0.488 | −0.489 | 0.014 | 0.247 | 0.809 | 0.641 | 0.247 | 0.309 |
| COX17 | 696533 \|\| [symbol] | 0.111 | −0.132 | 0.043 | −0.006 | −0.428 | −0.464 | −0.565 | 0.036 | 0.305 | 0.56 | 0.687 | 0.455 | 0.527 |
| COX5A | 690776 \|\| [symbol] | −0.327 | −0.058 | 0.431 | 0.418 | 0.415 | 0.274 | −0.084 | 0.214 | −0.415 | 0.57 | −0.211 | 0.07 | −0.368 |
| COX6A | 093143 \|\| [symbol] | −0.7105 | −0.7875 | −0.3155 | −0.4776 | −1.3315 | 0.3775 | −0.3655 | −0.9595 | −0.8305 | 0.8685 | 0.1675 | 1.1825 | 0.5795 |
| COX8A1 | 990654 \|\| [symbol] | 0.3025 | −0.3745 | −0.6765 | −0.7255 | 0.1835 | −0.1935 | 0.6755 | −0.5615 | −0.4815 | 0.5035 | 0.2266 | 0.7125 | 0.2365 |
| COX8C | 685589 \|\| [symbol] | 3.5955 | 6.2025 | −0.4325 | 1.6185 | 0.9995 | −1.4075 | −0.8555 | −1.2185 | −0.4025 | −1.4125 | 5.4585 | 3.4315 | 3.2515 |
| COX7A2 | 687312 \|\| [symbol] | 1.856 | 0.013 | 0.005 | −0.343 | −0.752 | −0.21 | 0.99 | −0.329 | −0.841 | −0.909 | 0.745 | 0.031 | 0.843 |
| CP | 883747 \|\| [symbol] | 0.2815 | −0.2295 | 1.3065 | 2.6975 | −0.5115 | −0.5115 | −0.2745 | −0.0685 | 1.8945 | −0.4895 | −0.1875 | −0.5305 | −0.1135 |
| CPAMD0 | 890973 \|\| [symbol] | −0.044 | −0.488 | −1.102 | 0.179 | −0.242 | −0.226 | 0.776 | −0.459 | 0.791 | 0.224 | −0.618 | −0.43 | −0.085 |
| CPB1 | 897093 \|\| [symbol] | 1.3635 | 0.3435 | −0.3955 | −0.0115 | −0.2295 | −1.2245 | 0.2905 | −0.5005 | −0.1415 | 0.6815 | −0.4525 | −0.1665 | −1.2275 |
| CPEB3 | 683341 \|\| [symbol] | −0.167 | −0.377 | −0.365 | 0.344 | 0.481 | 1.121 | 0.231 | 0.069 | 0.132 | −0.293 | 0.418 | 0.476 | 0.157 |
| CPEB4 | 681364 \|\| [symbol] | −0.406 | −0.564 | 0.197 | −0.871 | −0.285 | −0.803 | −0.948 | −0.374 | −0.001 | 0.127 | 0.852 | 0.282 | −0.679 |
| CPSF3 | 690199 \|\| [symbol] | 0.9835 | −0.3805 | −0.4715 | 0.8385 | 0.1505 | 0.3805 | 1.7075 | 0.1805 | 1.3555 | −0.2065 | 0.2255 | 0.5965 | 0.8535 |
| CRABP1 | 694973 \|\| [symbol] | −1.015 | −2.352 | 0.68 | 0.904 | −0.103 | −2.099 | −2.9 | 0.071 | 0.738 | 0.331 | 0.429 | −0.327 | 1.469 |
| CREB3L4 | 690183 \|\| [symbol] | −0.399 | 0.158 | 1.173 | 0.368 | 0.054 | −0.11 | −0.489 | 0.488 | 0.961 | 0.102 | 0.890 | 1.707 | −0.085 |
| CRIP1 | 866560 \|\| [symbol] | 1.228 | 0.289 | −0.397 | −0.089 | 0.023 | 0.297 | 0.181 | −0.146 | −1.18 | −0.021 | −0.507 | −0.242 | −0.177 |
| CRYBB2 | 883857 \|\| [symbol] | −0.495 | 0.213 | 0.59 | −1.334 | 0.16 | −1.489 | −1.154 | −0.68 | −0.985 | −0.954 | −0.883 | −1.201 | −0.889 |
| CRYL1 | 682598 \|\| [symbol] | 0.21 | 0.924 | 0.331 | −1.097 | 1.399 | −0.669 | −1.152 | 0.385 | −0.273 | 0.589 | −0.048 | −0.53 | 0.09 |
| CRYZ | 883831 \|\| [symbol] | −0.2245 | 0.0745 | −0.3865 | 0.1555 | −0.1425 | 0.4045 | 0.4485 | 0.1755 | 0.3915 | 0.8845 | 1.0315 | 0.3265 | 0.9685 |
| CSDA | 681102 \|\| [symbol] | −0.0625 | 0.0845 | −0.3535 | 0.1515 | −0.0665 | −0.1895 | −0.2195 | −0.1385 | −0.1745 | −0.2165 | −0.1725 | 0.3085 | 0.0765 |
| CSE1L | 893508 \|\| [symbol] | 0.0855 | −0.0495 | 0.7195 | 1.0055 | −0.0925 | −0.0385 | −0.7195 | 0.4825 | −0.0775 | −0.9675 | −0.7155 | 0.2215 | −1.2475 |
| CSK | 893320 \|\| [symbol] | 0.1495 | 0.7085 | 0.2335 | 0.3885 | −0.0745 | −0.6325 | −1.2525 | 1.8515 | 0.6255 | −1.4435 | 0.5655 | −0.4785 | 0.4805 |
| CSRP1 | 861688 \|\| [symbol] | 0.5135 | 0.9015 | 1.6916 | −0.9585 | 1.4945 | 1.3655 | −0.2075 | 1.2585 | 2.4505 | 1.5055 | 1.4265 | 1.3075 | 0.6335 |
| CSRP2 | 691158 \|\| [symbol] | 0.879 | 0.58 | −0.547 | −0.221 | −0.07 | 0.118 | 1.948 | −0.228 | −0.391 | 0.978 | 0.375 | 0.011 | 0.575 |
| CST5 | 888329 \|\| [symbol] | −0.4725 | 1.1435 | 0.5325 | 0.5195 | 0.2805 | 0.2785 | 0.1835 | 0.1325 | −0.7185 | 5.7045 | −0.5005 | 1.0815 | −0.2615 |
| CSTB | 888630 \|\| [symbol] | 0.008 | −0.038 | −0.054 | 0.28 | 0.579 | 0.078 | 0.446 | 0.001 | 0.387 | −0.862 | 0.145 | −0.093 | 0.303 |
| CTAG1B | 685110 \|\| [symbol] | −0.4885 | 0.1735 | 0.7645 | −0.6545 | 0.8185 | 0.1285 | 0.7315 | 0.5395 | 0.1845 | −0.3955 | −0.0205 | −1.2015 | 0.7485 |
| CTBP2 | 690880 \|\| [symbol] | 0.392 | 0.034 | 0.427 | −0.517 | 0.117 | 0.019 | 0.158 | 0.016 | −0.578 | −1.84 | −0.071 | −0.648 | 0.018 |
| CTNNA1 | 682257 \|\| [symbol] | 0.17 | −0.596 | 0.314 | 1.14 | −0.227 | −0.843 | −0.235 | −0.048 | 0.604 | −1.158 | −0.302 | 0.192 | −0.816 |
| CTNNAL1 | 683852 \|\| [symbol] | 0.3915 | 0.8545 | 0.5585 | −0.0315 | 0.8465 | 0.9995 | 0.4025 | 0.2835 | 0.2625 | 1.0665 | 0.4515 | 0.1445 | 0.3375 |
| CTNND2 | 592813 \|\| [symbol] | 0.228 | −0.072 | 1.124 | −1.08 | 0.159 | 0.177 | −0.165 | −0.124 | 0.273 | 0.895 | −0.023 | 0.004 | 0.928 |
| CTPS | 688832 \|\| [symbol] | −0.003 | 0.314 | 0.202 | −2.189 | −0.325 | 0.068 | −0.219 | 0.464 | −0.958 | 1.028 | 0.143 | 0.757 | −0.591 |
| CTSL2 | 596809 \|\| [symbol] | −0.1905 | 0.5306 | 0.2365 | 0.7625 | 0.4065 | 0.7125 | −0.5995 | −0.0255 | 1.1185 | −0.5935 | 0.6605 | 1.3635 | −0.0055 |
| CTSS | 688045 \|\| [symbol] | 1.91 | 1.337 | 4.048 | −0.722 | −1 | −1.81 | −0.91 | −0.73 | −1.2 | −0.45 | 1.57 | 0.749 | −1.93 |
| CTTN | 590810 \|\| [symbol] | −0.2625 | 0.9685 | −1.5385 | −1.4365 | −1.3185 | 1.5635 | 1.5015 | −1.1225 | −0.7825 | 2.4876 | 0.7195 | −0.1335 | 0.9275 |
| CX3CL1 | 762475 \|\| [symbol] | 0.4075 | −0.1855 | −0.1545 | 0.3415 | 1.1925 | 0.8255 | 0.5465 | −0.4855 | 0.6405 | 0.2895 | 0.3565 | 0.0395 | −0.6305 |
| CXCL9 | 694763 \|\| [symbol] | 0.0145 | −1.2435 | −0.5195 | 1.9915 | 1.3475 | 1.7325 | −0.2595 | 0.2205 | 0.6755 | −0.5915 | −0.7025 | −0.1605 | 0.2635 |
| CXorf39 | 582198 \|\| [symbol] | 1.241 | 0.217 | 0.116 | −0.788 | 1.349 | −0.911 | −0.225 | 0.018 | −0.199 | 2.018 | 1.049 | 0.407 | 0.112 |
| CXXC4 | 884240 \|\| [symbol] | 1.371 | −0.018 | −0.267 | 0.343 | 0.286 | 2.047 | 0.281 | 0.018 | −0.208 | 1.63 | −0.081 | −0.3 | 0.76 |
| CYB5 | 590445 \|\| [symbol] | 0.1995 | 0.6615 | −0.6215 | 0.3385 | −0.5625 | 0.0025 | −0.1965 | 0.8995 | −0.5915 | 1.5615 | 1.5705 | 2.0105 | 1.3255 |
| CYB561 | 583887 \|\| [symbol] | −0.088 | 0.827 | −0.96 | 0.845 | −0.9 | −0.017 | −1.163 | 0.427 | −0.83 | 0.08 | 0.266 | 1.345 | 1.068 |
| CYC1 | 683057 \|\| [symbol] | −0.06 | −0.144 | 0.469 | 0.165 | −0.207 | 0.007 | 0.118 | −0.005 | −0.141 | −0.027 | 0.093 | 0.228 | 0.315 |
| CYHR1 | 697209 \|\| [symbol] | 1.3475 | 3.7785 | 0.4365 | 0.3365 | 2.5955 | −1.0835 | 2.9685 | −1.3115 | −1.3535 | −1.2585 | 1.5705 | 0.4745 | −0.9185 |
| CYP2A13 | 689236 \|\| [symbol] | 0.166 | −0.067 | 0.68 | 0.065 | 0.211 | −0.307 | −0.113 | 0.183 | −0.473 | 0.94 | 0.211 | −0.051 | −0.038 |
| CYP4X1 | 681940 \|\| [symbol] | −0.6395 | −1.6235 | −0.9715 | −0.2275 | −1.2475 | 0.3725 | −2.3075 | 0.0595 | 0.3955 | −1.9985 | −0.7035 | −0.3515 | −2.1085 |
| D15Wsu7 [symbol] | 684616 \|\| [symbol] | −0.6695 | −0.8495 | −0.5905 | 0.0125 | −0.9725 | 0.0995 | −1.1005 | −0.1655 | 0.0285 | −0.9465 | −0.6985 | −0.3345 | −0.9705 |
| DACH1 | 892996 \|\| [symbol] | −0.22 | −1.529 | −0.825 | −0.171 | −1.798 | 0.721 | −2.258 | 0.24 | 0.120 | −1.562 | −0.798 | −0.097 | −2.042 |
| DACH1 | 694269 \|\| [symbol] | −0.458 | −0.311 | −0.136 | −0.43 | −0.311 | 0.228 | 0.619 | −0.268 | −0.013 | −0.698 | 0.124 | −0.106 | 0.12 |
| DACH1 | 597247 \|\| [symbol] | −0.124 | −0.66 | 0.483 | −0.912 | 0.27 | −0.418 | −0.157 | −0.134 | 0.085 | 0.349 | −0.094 | −0.239 | −0.100 |
| DAP3 | 089542 \|\| [symbol] | −0.5145 | −0.2455 | 0.6405 | 0.0145 | −0.0865 | −0.3775 | −0.4455 | 0.1855 | −0.2445 | −0.8285 | −0.2285 | −0.2185 | −0.8595 |
| DAPP1 | 681876 \|\| [symbol] | 1.057 | −0.308 | −0.458 | 1.397 | 1.047 | 0.048 | 2.174 | −0.982 | −0.251 | 2.08 | 1.022 | 1.029 | 1.15 |
| DAZAP2 | 666305 \|\| [symbol] | 1.595 | 0.763 | 0.487 | 0.244 | 0.558 | 1.858 | 0.117 | 0.785 | 0.41 | −0.502 | 0.012 | −0.263 | 0.277 |
| DBI | 697444 \|\| [symbol] | 0.5735 | 0.1525 | 0.7255 | −0.9855 | 0.4035 | 0.3515 | 0.0485 | 0.2435 | −0.5235 | 0.3055 | 0.3435 | 0.1575 | 0.1075 |
| D9N1 | 985238 \|\| [symbol] | −0.343 | 0.087 | −0.807 | −0.639 | −0.592 | 0.312 | −0.092 | −0.258 | −0.312 | 0.892 | 0.555 | 0.708 | 0.593 |
| DC13 | [symbol] 93725 \|\| [symbol] | −0.2175 | 0.2775 | −0.9715 | −0.1065 | −0.1555 | 0.6075 | 0.7455 | 0.1655 | 0.2065 | 0.2505 | 0.7375 | 1.1155 | −1.0705 |
| DCPS | 683083 \|\| [symbol] | −0.3585 | 0.7205 | −1.3255 | 0.4215 | 0.7855 | −0.4745 | −0.5485 | 1.4485 | 0.5485 | 0.8165 | 0.3325 | −0.0015 | 0.7615 |
| DDHD2 | 683946 \|\| [symbol] | −0.4855 | −0.2345 | −0.2455 | −0.1475 | 0.5945 | 0.3235 | 0.0039 | 0.1345 | 0.2535 | 0.6555 | 0.6745 | 0.4135 | 0.6815 |
| D0X17 | 694961 \|\| [symbol] | −0.9225 | −0.0105 | 0.0015 | −0.7445 | 0.5895 | 0.8805 | 0.2905 | 0.0395 | −1.2365 | 0.0595 | −0.0725 | −0.7215 | 1.5125 |
| DDX39 | 681303 \|\| [symbol] | −1.358 | −0.375 | 1.149 | 0.453 | −1.382 | −1.253 | −0.037 | 0.784 | 2.001 | −1.473 | 1.202 | 1.764 | 0.415 |
| DDX48 | 696985 \|\| [symbol] | −0.2255 | −0.4285 | −0.5175 | 0.2785 | −1.6635 | −0.5825 | 0.0985 | −0.0715 | −0.4245 | 0.3765 | −0.6555 | −0.4285 | −0.2855 |
| DDX58 | 691620 \|\| [symbol] | −0.3355 | 0.2455 | 0.2785 | −1.8015 | −0.4455 | −0.5945 | −0.5435 | 0.0876 | −0.2495 | 0.0995 | 0.2755 | −0.9305 | −0.1245 |
| DEF6 | 582749 \|\| [symbol] | 0.653 | 0.505 | 0.255 | 0.893 | 0.815 | −0.493 | 0.598 | −0.094 | 1.876 | 0.948 | 0.907 | 0.649 | 0.501 |
| DEK | 688515 \|\| [symbol] | −0.84 | 0.583 | −0.441 | 0.583 | 0.279 | −0.288 | −0.942 | 0.984 | 1.045 | −0.294 | −0.538 | −0.476 | −0.974 |
| DHCR7 | 994109 \|\| [symbol] | 1.791 | 1.328 | −0.776 | 1.752 | 1.797 | −0.533 | 1.916 | −0.042 | 0.451 | 0.443 | 2.121 | 1.293 | 1.228 |
| DHRS7 | 682438 \|\| [symbol] | −0.5335 | −0.8465 | −0.2215 | −0.0185 | −1.3305 | −1.3355 | −0.2235 | −1.3785 | 0.2575 | 0.8575 | −0.1685 | −0.4935 | −0.1495 |
| DHX57 | 688263 \|\| [symbol] | 3.8465 | −1.7435 | −0.9575 | 2.8735 | 2.7995 | −0.0875 | 3.5935 | 0.9625 | 0.5315 | 3.4515 | −0.8585 | 0.3795 | 4.8185 |
| DIRC1 | 991337 \|\| [symbol] | 0.011 | 0.251 | 0.259 | −0.09 | 1.304 | 0.414 | −0.876 | −0.578 | −0.218 | −0.232 | 0.131 | 0.291 | 0.069 |
| dJ222E13 | 989427 \|\| [symbol] | 0.005 | −0.791 | 0.393 | 0.12 | 0.572 | −0.193 | −0.321 | −0.118 | −1.015 | −0.012 | −0.288 | −0.599 | −1.239 |
| DKFZP434 | 689930 \|\| [symbol] | 0.88 | 0.446 | 0.234 | 1.331 | 0.788 | 0.738 | 1.765 | 0.612 | 0.537 | 0.8 | 0.391 | 0.926 | 1.319 |
| DKFZp434 | 689808 \|\| [symbol] | −0.3465 | 0.3175 | 0.0565 | 0.4375 | −0.4525 | 1.0225 | 1.2755 | 0.0885 | 0.2525 | 0.7345 | 0.6085 | 0.8045 | 1.5795 |
| DKFZP434 | 881071 \|\| [symbol] | −0.279 | 0.015 | 0.85 | −0.05 | −0.922 | −0.203 | 0.143 | −0.016 | −0.36 | −0.292 | −0.518 | 0.181 | −1.376 |
| DKFZp434 | 683495 \|\| [symbol] | −0.2915 | −0.5885 | 0.1035 | 0.3495 | 0.3885 | −0.1035 | −0.4385 | 0.1905 | 0.3505 | −0.5215 | −0.1385 | 0.3455 | −0.6065 |
| DKFZP564 | 684976 \|\| [symbol] | 0.0395 | 0.4505 | −0.4015 | 0.7885 | 1.2535 | −0.0875 | 0.0625 | 0.0416 | −0.1355 | 0.7835 | −0.2265 | 0.3735 | 0.2715 |
| DKFZp564 | 897724 \|\| [symbol] | −0.442 | 0.518 | 0.994 | −0.763 | −0.029 | 1.027 | −0.089 | 0.875 | −0.745 | 2.398 | 1.239 | 0.779 | 0.907 |
| DKFZP564 | 683858 \|\| [symbol] | −0.0815 | −0.0235 | 0.4805 | −0.0105 | −0.5305 | 0.2485 | 0.4375 | −0.3415 | −0.2495 | −0.0095 | −0.5785 | −0.5345 | −0.2075 |
| DKFZP564 | 688875 \|\| [symbol] | −0.196 | 0.014 | −0.374 | 0.317 | −0.292 | −0.15 | −0.471 | −0.029 | 0.346 | 0.605 | 0.729 | 0.824 | 0.224 |
| DKFZP564 | 682674 \|\| [symbol] | −1.13 | −1.071 | −0.178 | −0.813 | 0.655 | −0.163 | 0.514 | −0.68 | −1.002 | −0.919 | −0.536 | −1.874 | −0.021 |
| DKFZp586 | 895204 \|\| [symbol] | 0.4465 | −0.1975 | 0.6115 | 4.5745 | 1.2495 | 0.0395 | 2.3505 | −0.4195 | −0.6035 | −0.1195 | 0.1565 | 1.0855 | 0.4535 |
| DKFZp761 | 881865 \|\| [symbol] | −0.281 | −0.029 | 0.722 | 0.336 | −0.081 | −0.091 | −0.415 | 0.123 | −0.23 | −0.093 | 0.099 | −0.115 | 0.149 |
| DLK1 | 665295 \|\| [symbol] | 0.571 | 0.56 | −0.163 | 0.352 | 0.017 | −0.107 | 1.008 | 0.504 | 0.721 | 1.243 | 0.131 | 0.402 | 0.902 |
| DLX5 | 682457 \|\| [symbol] | 0.1075 | −0.0875 | −0.0085 | 0.9675 | 1.1675 | 0.5025 | 1.2926 | 1.2505 | 0.4495 | 1.3665 | −0.1805 | 0.1005 | 1.1015 |
| DNAJA4 | 681226 \|\| [symbol] | −0.6685 | −1.7405 | −1.8585 | −0.1515 | 0.0165 | −1.7565 | −2.3355 | −0.9655 | 1.0675 | −3.1535 | 1.2805 | 0.4075 | −1.5585 |
| DNAJC1 | 692749 \|\| [symbol] | 0.295 | 0.468 | −1.195 | 0.18 | 2.458 | 0.826 | −0.311 | 0.525 | −1.197 | 1.322 | 1.205 | 0.732 | 1.438 |
| DNAJC12 | 696427 \|\| [symbol] | −1.516 | −1.728 | 0.258 | −0.927 | −3.152 | 0.651 | −1.705 | −0.635 | −1.722 | −2.681 | 1.198 | 0.623 | −2.858 |
| DNAJD1 | 690353 \|\| [symbol] | −0.578 | −0.62 | −0.565 | −0.618 | 0.031 | 0.18 | 0.694 | −0.399 | −0.003 | −0.11 | 0.095 | −0.45 | 0.073 |
| DNALI1 | 695070 \|\| [symbol] | 1.764 | −0.01 | −0.007 | −0.598 | 0.171 | 0.031 | 0.598 | 0.438 | 0.474 | 0.549 | −0.058 | −0.956 | −0.506 |
| DNMT1 | 681128 \|\| [symbol] | 0.6215 | 0.5395 | 0.5915 | 0.5745 | −1.3745 | −1.1245 | −0.4845 | 0.1135 | −0.2685 | 1.9935 | −0.0205 | −0.2455 | −0.2735 |
| DOX1 | 597175 \|\| [symbol] | −0.1585 | 0.0745 | 0.4045 | 1.2115 | −1.0995 | 0.4265 | 0.2765 | −0.8835 | −0.6885 | 0.6735 | 0.1785 | 0.1105 | −0.5945 |
| DSCR1 | 590387 \|\| [symbol] | −0.2585 | −0.1145 | 0.4085 | −0.9095 | 0.6415 | −0.1485 | −0.6825 | −0.3085 | −0.2005 | 0.0275 | 0.0995 | −1.2825 | −0.7465 |
| DSCR1L2 | 885798 \|\| [symbol] | −0.0075 | −0.1355 | −0.2255 | −0.2935 | 0.0835 | 0.2415 | 0.9645 | −0.2305 | −0.1095 | 0.6995 | 0.4905 | 0.5725 | 0.4225 |
| DSP | 895661 \|\| [symbol] | 0.8735 | 0.3875 | −0.0175 | −0.4305 | −1.3225 | 0.0755 | 0.8295 | −0.5765 | −0.1305 | −0.2585 | −0.3185 | 0.2475 | −0.6465 |
| DTYMK | 882833 \|\| [symbol] | −0.1125 | −0.3925 | 0.9095 | 1.3925 | 0.6085 | 0.8845 | 1.7295 | 0.8465 | 1.9005 | 0.7785 | −0.1565 | 1.0135 | 0.8825 |
| DUSP23 | 691997 \|\| [symbol] | 1.9165 | 0.4485 | 0.3775 | 1.7455 | 2.3339 | 0.6285 | 1.3855 | 0.8345 | 0.2835 | −0.9865 | −0.7805 | 0.3785 | −0.4345 |
| DUSP4 | 693976 \|\| [symbol] | 0.3635 | 0.9045 | 1.0155 | 0.2105 | 0.5425 | −0.1685 | 0.5905 | −0.5685 | −0.4135 | 0.4285 | 0.2085 | 0.0375 | 0.2115 |
| DUSP5 | 693200 \|\| [symbol] | −0.442 | 0.111 | 0.273 | 0.01 | 0.193 | 0.152 | −0.053 | −0.001 | 0.209 | 0.250 | −0.018 | 0.374 | 0.194 |
| DVL1 | 582613 \|\| [symbol] | 0.125 | −0.585 | −0.159 | 0.898 | −0.189 | 0.401 | −0.706 | 0.366 | 0.123 | −0.03 | −0.262 | 0.523 | −1.229 |
| DXYS155 [symbol] | 894208 \|\| [symbol] | −0.273 | 0.728 | 1.483 | −0.318 | −0.158 | 0.724 | 0.018 | −0.232 | −0.549 | 0.795 | 1.242 | 0.848 | 1.199 |
| DYRK4 | 807043 \|\| [symbol] | 1.3125 | −0.4335 | −0.8525 | 1.6315 | 1.4595 | 1.8605 | 1.2925 | −0.0025 | 0.5675 | 1.0185 | 0.3585 | −0.6595 | 1.3755 |
| o(y)2 | 695983 \|\| [symbol] | −0.119 | −0.344 | 1.755 | 0.685 | −0.292 | 2.709 | −0.232 | 0.213 | 0.137 | 0.008 | −0.495 | −0.128 | 1.705 |
| EAF2 | 690585 \|\| [symbol] | 0.2239 | 1.3685 | −0.5685 | −0.6415 | −1.7505 | −0.8265 | −1.5615 | −0.5945 | −1.5435 | −0.0155 | −0.5005 | −1.8575 | −0.8255 |
| EAP30 | 695304 \|\| [symbol] | 0.1695 | −0.5715 | −2.6135 | −0.6145 | −0.8135 | −1.4415 | 0.4245 | −1.5335 | −1.4535 | 1.0095 | −0.4155 | 0.5405 | 0.6125 |
| ECHDC1 | 695058 \|\| [symbol] | 0.5455 | 0.5295 | 0.1285 | −0.3375 | −0.2745 | −0.1805 | 1.2225 | 0.5995 | 0.5555 | −1.7095 | −0.0565 | 0.7335 | 0.0425 |
| ECHDC3 | 693484 \|\| [symbol] | −0.2565 | −0.1685 | −0.7125 | 0.8405 | −0.2825 | 0.9369 | −0.6439 | −0.3405 | −0.1385 | −1.1185 | −0.8395 | −0.5095 | −1.1305 |
| EEF1A2 | 898775 \|\| [symbol] | 0.511 | 0.529 | −0.778 | 0.47 | 1.52 | 1.208 | 1.83 | −0.041 | −0.082 | −1.257 | −0.187 | 0.254 | 0.428 |
| EFHC1 | 694066 \|\| [symbol] | 0.3145 | 0.4175 | −0.5015 | −0.1145 | 0.6585 | −0.3285 | 0.2865 | 0.0115 | 0.8605 | 0.2595 | 0.0055 | −0.0765 | −0.4355 |
| EFNA4 | 687429 \|\| [symbol] | −0.1465 | 0.9485 | −0.0215 | 0.0355 | 0.7235 | 0.9525 | −0.3525 | 0.5855 | 0.0055 | 0.7075 | 1.2905 | 1.4145 | 1.1835 |
| EFS | 885583 \|\| [symbol] | 0.0155 | 0.8305 | 0.1325 | −0.2785 | −0.4015 | −0.5845 | −0.2345 | −0.1915 | 0.0085 | −0.5095 | −0.7755 | −0.8295 | −0.0785 |
| EIF2C2 | 698794 \|\| [symbol] | −0.8555 | 0.0315 | 0.8085 | 1.2985 | 0.2106 | 0.1285 | 0.2055 | 0.0295 | 0.7115 | 1.6006 | 1.3045 | 1.1015 | 0.2775 |
| EIF2C4 | 689728 \|\| [symbol] | 0.826 | 0.482 | 0.497 | 0.422 | 0.271 | 0.636 | 1.253 | 0.104 | 0.249 | 0.221 | −0.014 | 0.288 | −0.053 |
| EIF4EBP1 | 883217 \|\| [symbol] | 0.5725 | 0.3475 | 0.0135 | 0.0575 | −0.6345 | 0.1985 | 0.4235 | −0.1845 | −0.1845 | −0.3275 | 0.2635 | −0.1195 | 0.4235 |
| EIF4G1 | 562045 \|\| [symbol] | −0.047 | 0.237 | −0.85 | −0.503 | −1.345 | 0.045 | −0.444 | −0.193 | −0.241 | 0.019 | −0.548 | −0.281 | −1.577 |
| EIF4G3 | 683768 \|\| [symbol] | −0.411 | 0.484 | 1.126 | −0.541 | −0.162 | −0.272 | 0.001 | 0.318 | 0.924 | 1.178 | 0.271 | 0.374 | 0.14 |
| ELAC1 | 695486 \|\| [symbol] | 0.613 | −0.402 | 0.309 | 1.707 | −0.11 | −0.155 | 0.561 | −0.442 | 0.308 | 2.112 | −0.485 | −0.574 | 1.081 |
| ELL2 | 884068 \|\| [symbol] | 0.7395 | 0.0865 | −0.5445 | −1.5725 | 0.8585 | −0.1195 | 0.8575 | −0.8165 | −0.8085 | −0.4945 | −0.4615 | −0.0475 | 0.2205 |
| ELL3 | 695507 \|\| [symbol] | −0.2385 | 1.2305 | 0.0855 | −0.0135 | −0.5265 | 0.2325 | −0.0005 | 0.1415 | 0.0915 | 0.5385 | 1.0595 | 1.1735 | −0.2195 |
| ELOVL5 | 694593 \|\| [symbol] | 0.4445 | 0.5845 | 0.2055 | 0.3445 | 0.8915 | 1.1625 | 0.9925 | 0.7185 | 0.3855 | −0.1425 | 0.2085 | −0.3825 | −0.5325 |
| EN1 | 689064 \|\| [symbol] | −0.2476 | −0.9355 | −1.4835 | −0.0285 | −0.0305 | −0.8285 | 0.7475 | −1.3565 | −0.7255 | 0.5405 | −0.9505 | −0.7905 | −0.5895 |
| ENAH | 682255 \|\| [symbol] | 0.7035 | 0.1845 | 0.0035 | −0.2265 | −0.0925 | 0.7645 | −0.2085 | 1.0475 | 0.5095 | −0.8235 | −0.7365 | −0.4055 | −0.3805 |
| EPHX2 | 883125 \|\| [symbol] | −0.499 | −0.912 | 3.181 | 1.237 | 0.331 | 1.638 | 1.757 | 0.416 | −0.012 | 0.012 | −0.005 | 2.073 | 0.218 |
| EPLIN | 687810 \|\| [symbol] | −0.2505 | −0.3905 | −1.1085 | 0.0115 | 0.4505 | −0.1425 | 0.8035 | 0.8705 | −1.0105 | −0.7895 | −0.7425 | −0.6045 | −0.0725 |
| EPN3 | 581782 \|\| [symbol] | 4.1095 | 3.2945 | 3.8875 | 3.5725 | 3.7015 | 3.8895 | −0.0005 | 3.0645 | 4.6425 | 2.4935 | 2.4535 | 1.5025 | 4.2895 |
| EPOR | 684991 \|\| [symbol] | 0.089 | 0.413 | 0.095 | 0.221 | −0.088 | 0.253 | 1.513 | −0.076 | 0.595 | −0.174 | 0.339 | −0.327 | 0.843 |
| ERBB2 | 888427 \|\| [symbol] | −2.7985 | −1.5865 | −1.6605 | −1.2655 | −3.6665 | −5.0595 | −3.8875 | −4.3535 | 1.4745 | −4.8575 | −2.5675 | −0.8885 | −4.8715 |
| ERP70 | 690269 \|\| [symbol] | 1.194 | 0.715 | −1.633 | −0.224 | −0.004 | 0.825 | 1.103 | 0.023 | 0.258 | 1.766 | 0.953 | 0.038 | 0.199 |
| ESR1 | 765041 \|\| [symbol] | 0.0815 | 0.3955 | −0.2255 | −0.4475 | −0.1005 | 0.3045 | −0.3015 | −0.0765 | −0.4485 | −0.6655 | −0.0935 | −0.2136 | 0.4455 |
| ETFA | 693087 \|\| [symbol] | 0.3185 | 0.4725 | 0.2195 | −0.2685 | 1.0335 | 1.8915 | 1.0785 | −0.5995 | −0.4775 | 0.3455 | 0.3915 | 0.7615 | 1.8945 |
| ETV8 | 697286 \|\| [symbol] | −0.0385 | −0.0635 | −0.2835 | −0.4435 | −0.3285 | 1.2465 | 1.4465 | −0.0835 | 0.4445 | 1.4315 | 0.8395 | 0.4265 | 0.4945 |
| EVER1 | 688220 \|\| [symbol] | −0.5115 | −0.5225 | 0.5495 | −0.0205 | 0.2015 | 0.4655 | −0.5845 | 0.3655 | −0.0475 | 0.1645 | 0.3505 | 0.5355 | −0.0145 |
| EXO1 | 889742 \|\| [symbol] | −0.2435 | 0.3145 | −0.1785 | −0.7345 | −0.3745 | 0.8525 | 0.4215 | 0.8995 | 0.5725 | −0.0405 | −0.4845 | −0.9805 | 0.6285 |
| EXOSC9 | 591275 \|\| [symbol] | 0.126 | −0.32 | −0.027 | −0.47 | 0.928 | 1.04 | 1.204 | 0.524 | 0.226 | 0.293 | −0.046 | −0.372 | 0.335 |
| EXT2 | 693031 \|\| [symbol] | −0.045 | −0.129 | 0.528 | 0.465 | −2.128 | −0.228 | 0.218 | −1.088 | −0.526 | −0.771 | −1.449 | −0.677 | −0.308 |
| EZH2 | 689533 \|\| [symbol] | −0.1805 | −0.3685 | 0.4455 | 0.6355 | 0.2115 | −0.0625 | 0.3815 | −0.5375 | −0.1195 | −0.3416 | 0.4595 | −0.2815 | 0.2045 |
| FI1R | 589081 \|\| [symbol] | 3.114 | 0.141 | 0.537 | 1.347 | 1.668 | −0.907 | 1.789 | 0.745 | 1.14 | 1.456 | 0.221 | 1.281 | 2.14 |
| F [symbol] | 692838 \|\| [symbol] | 0.509 | 0.515 | 1.053 | 5.872 | 3.834 | −0.07 | 7.018 | −0.17 | 0.149 | 1.054 | −0.78 | 1.585 | 5.001 |
| FA2H | 888063 \|\| [symbol] | 0.284 | 0.221 | 0.328 | −0.293 | 0.548 | 1.23 | 0.108 | 0.438 | 0.174 | −0.815 | 0.282 | 0.138 | −0.545 |
| FABP7 | 694559 \|\| [symbol] | −0.657 | −0.173 | −0.086 | −0.519 | 0.273 | 0.115 | −0.484 | 0.494 | −0.455 | 1.137 | −0.277 | −0.046 | −0.443 |
| FADD | 886191 \|\| [symbol] | 0.09 | 0.499 | −0.353 | 0.775 | −0.769 | 0.031 | 0.275 | −0.708 | −0.452 | 0.33 | 0.326 | 0.283 | 0.363 |
| FAHD1 | 688442 \|\| [symbol] | 0.09 | 0.207 | −0.09 | 0.084 | 0.092 | 0.277 | 0.384 | −0.514 | 0.048 | 1.104 | 0.318 | 0.144 | 0.356 |
| FAHD2A | 687680 \|\| [symbol] | 0.236 | 0.564 | −0.614 | 0.618 | 0.382 | 0.018 | 0.51 | 0.092 | −0.15 | 0.018 | 0.051 | 0.728 | −0.485 |
| FAM38A | 683002 \|\| [symbol] | −0.9485 | −0.8185 | −0.5445 | 2.8825 | −1.3815 | −0.5805 | 1.7585 | −0.2435 | −0.2585 | −0.8475 | −0.6255 | −1.4235 | −0.0705 |
| FAM3A | 695471 \|\| [symbol] | 0.1405 | 0.7015 | 0.4825 | −1.2535 | −0.9305 | 0.5345 | 0.3295 | 0.6855 | 0.7405 | 0.7725 | 0.1785 | −0.2655 | −0.0255 |
| FAM3B | 685659 \|\| [symbol] | −0.819 | −0.603 | −0.844 | −0.181 | −0.351 | −0.414 | 0.558 | −0.748 | 0.265 | −0.352 | −0.283 | 0.835 | 0.345 |
| FAM3C | 681823 \|\| [symbol] | −0.414 | 0.937 | 0.425 | −0.783 | −0.456 | 0.024 | 0.732 | 1.477 | −1.313 | −0.099 | 1.559 | 0.711 | 0.838 |
| FAM46C | 685310 \|\| [symbol] | −0.444 | −0.053 | −0.588 | −0.976 | −0.23 | 0.562 | 1.322 | −0.12 | 0.43 | 1.553 | 1.398 | 1.107 | 0.375 |
| FAM49B | 692836 \|\| [symbol] | 0.343 | 0.697 | −0.493 | −0.083 | −0.701 | 0.817 | 1.194 | −0.321 | −0.454 | 0.038 | 0.292 | 0.527 | 1.649 |
| FAM54A | 890964 \|\| [symbol] | −0.451 | −0.424 | −0.508 | −0.507 | −0.573 | −0.171 | 0.83 | −0.251 | −0.422 | 0.139 | 0.506 | −0.493 | 1.008 |
| FANCA | 685878 \|\| [symbol] | −0.1955 | −0.2375 | 1.1855 | −0.2345 | −1.0055 | 0.1605 | −0.8055 | −0.1845 | 0.0325 | −0.5855 | 0.0235 | 0.0865 | 0.3505 |
| FANCE | 892748 \|\| [symbol] | 0.3725 | −1.4485 | −0.3515 | −0.5535 | −1.2115 | −1.7555 | −0.2015 | −1.3565 | 0.3395 | −1.7976 | −1.9285 | −0.3255 | 0.8825 |
| FBL | 882815 \|\| [symbol] | 0 | 0.245 | −0.062 | 0.303 | 0.204 | −0.081 | −0.127 | −0.057 | 0.17 | −0.207 | −0.139 | −0.073 | −0.134 |
| FBP1 | 587231 \|\| [symbol] | −0.881 | 0.038 | −0.160 | −0.403 | 0.934 | 0.044 | −0.019 | 0.918 | 0.009 | 0.118 | 0.783 | 0.515 | 0.083 |
| FBS1 | 683642 \|\| [symbol] | −0.1825 | −0.8015 | 0.1515 | 0.8765 | 0.8315 | 0.0765 | 0.7035 | 0.1125 | −0.1205 | −0.5585 | −0.4065 | 0.1625 | −0.0455 |
| FBXL20 | 695291 \|\| [symbol] | −0.415 | −0.399 | −0.017 | −0.078 | 0.079 | −0.022 | 0.194 | 0.097 | −0.47 | −0.303 | −0.478 | 0.023 | 2.085 |
| FBXO25 | 882373 \|\| [symbol] | 0.059 | 0.547 | 0.529 | −0.314 | 0.705 | −0.17 | −0.189 | 0.49 | 0.469 | 0.258 | 0.332 | 0.033 | 0.142 |
| FDXR | 698354 \|\| [symbol] | 2.358 | −0.412 | 0.846 | 1.552 | 2.322 | 0.106 | 1.582 | 0.895 | 1.003 | 0.059 | −0.491 | −0.543 | −0.525 |
| FEM1B | 887319 \|\| [symbol] | 0.91 | 1.32 | −0.315 | 2.169 | 1.307 | 1.173 | 1.193 | 0.081 | 0.049 | −1.014 | −0.211 | 0.007 | 0.328 |
| FGF12 | 886589 \|\| [symbol] | 1.8085 | 1.8245 | −0.0145 | 0.3995 | 1.6455 | 0.7375 | 0.8415 | 0.5585 | 0.5195 | 0.2995 | 0.5135 | 0.8575 | 1.0435 |
| FGF13 | 895768 \|\| [symbol] | 0.914 | −0.431 | −0.539 | −0.207 | 0.232 | −0.485 | 0.047 | −0.259 | 0.591 | −0.297 | 0.869 | −0.048 | −0.12 |
| FHOD1 | 693587 \|\| [symbol] | 0.3405 | −0.1195 | 1.0785 | 0.1825 | −0.2395 | 0.0845 | 0.9435 | 0.2625 | 0.8665 | 0.4855 | 0.3005 | −0.0335 | −0.2095 |
| FKBP11 | 684453 \|\| [symbol] | −0.2955 | −0.5835 | −1.1625 | −0.0385 | −1.0505 | −1.1385 | −0.0625 | −1.3885 | 0.3785 | 0.3325 | 0.0545 | 0.4565 | −0.3065 |
| FLJ10094 | 684307 \|\| [symbol] | 0.829 | 0.583 | −0.211 | 0.005 | −0.386 | 0.152 | −0.343 | 0.837 | −0.005 | −0.784 | −0.447 | −0.928 | −0.789 |
| FLJ10115 | 682368 \|\| [symbol] | −0.3945 | 0.5185 | 0.0065 | −0.7705 | −0.3335 | −0.0285 | 0.2065 | 0.4745 | −0.5295 | 0.6585 | 1.3485 | 1.4965 | 0.9285 |
| FLJ10159 | 885392 \|\| [symbol] | 0.689 | 0.103 | 0.271 | 1.066 | −0.473 | 0.332 | 0.826 | 0.317 | 0.033 | 0.019 | −0.595 | −0.343 | −0.144 |
| FLJ10204 | 688075 \|\| [symbol] | −0.343 | −0.157 | −0.346 | −0.451 | 0.38 | 0.543 | 0.28 | 0.055 | 0.794 | 0.518 | −0.451 | −0.284 | −0.204 |
| FLJ10275 | 694528 \|\| [symbol] | 0.9795 | −0.0585 | −1.3165 | 1.7775 | −1.0455 | 1.8845 | 1.8975 | 0.3745 | −0.5885 | 0.7655 | −1.0345 | −0.8325 | −1.5665 |
| FLJ10292 | 666906 \|\| [symbol] | 0.7355 | −0.6185 | −1.0185 | 1.8705 | 1.7875 | 1.0065 | 0.1085 | −1.1615 | −0.1925 | −0.4775 | 0.1035 | 0.4315 | 2.4205 |
| FLJ10324 | 893717 \|\| [symbol] | −0.6415 | −0.6235 | −0.2595 | 0.2905 | −0.4355 | 0.1435 | 1.2075 | −0.0585 | 0.0135 | −0.1645 | 0.6635 | 0.8705 | 0.8955 |
| FLJ10700 | 887078 \|\| [symbol] | −0.164 | −0.423 | −0.537 | 0.508 | 0.905 | 0.274 | 0.468 | −0.128 | 0.332 | −0.376 | −0.86 | −0.69 | −0.413 |
| FLJ10706 | 693300 \|\| [symbol] | 1.895 | 0.74 | 0.468 | 0.147 | 2.384 | 1.734 | 2.842 | −0.018 | 0.103 | 0.288 | 1.061 | 1.35 | 0.917 |
| FLJ10671 | 694856 \|\| [symbol] | −0.283 | 0.545 | 0.067 | 0.605 | 0.459 | 0.514 | −0.823 | −0.611 | −0.493 | −1.747 | −0.208 | 0.099 | 0.069 |
| FLJ10901 | 684051 \|\| [symbol] | 0.6345 | −0.4395 | 0.4245 | 0.5195 | 2.7455 | −0.4775 | 0.6715 | −0.4885 | 0.1985 | 0.2865 | −0.5605 | −0.2725 | 0.2405 |
| FLJ10918 | 695840 \|\| [symbol] | 0.108 | 1.441 | 0.332 | −0.84 | −0.967 | −0.132 | 0.015 | −0.554 | −0.635 | −0.784 | −0.258 | −0.164 | −0.254 |
| FLJ10980 | 684797 \|\| [symbol] | −0.6265 | −0.4925 | −0.0035 | −0.0825 | −0.3215 | −0.8515 | −0.1645 | −0.0805 | 0.3565 | −0.0855 | −0.5055 | −0.5985 | −0.9405 |
| FLJ11017 | 882528 \|\| [symbol] | 0.4225 | 1.1295 | −0.1535 | 0.3595 | −0.3205 | 0.6995 | 0.4325 | 0.4045 | 0.1885 | 1.7745 | 1.3505 | 0.7125 | 0.4955 |
| FLJ11088 | 689859 \|\| [symbol] | 0.1705 | 0.4955 | −0.3305 | −0.0625 | −0.3185 | 0.1565 | −0.5775 | −0.0015 | −0.9055 | −0.2555 | −0.6025 | −0.9216 | 0.4325 |
| FLJ11151 | 695680 \|\| [symbol] | 0.0135 | −0.3665 | 0.2905 | 0.3095 | 0.4385 | −0.2775 | −0.3965 | −0.7735 | −0.0535 | −1.1345 | −0.0135 | 0.6725 | −0.3745 |
| FLJ11267 | 688538 \|\| [symbol] | −0.0405 | −0.1405 | 0.2505 | −0.6625 | −0.2655 | 0.1995 | −0.3885 | −0.0225 | 0.5125 | −0.3065 | 0.2525 | 0.4855 | −0.1015 |
| FLJ11280 | 697294 \|\| [symbol] | 0.8765 | 0.4065 | −0.8235 | −0.8905 | 0.0475 | 0.5715 | 0.0385 | 0.0125 | −0.4455 | 0.3015 | −0.5915 | −0.8595 | −0.1276 |
| FLJ11508 | [symbol] 912 [symbol] 5 \|\| [symbol] | −0.4415 | 0.6425 | −0.8415 | 0.3615 | −0.9585 | 0.4875 | 0.0315 | 0.8235 | −0.3465 | −1.3045 | 0.0225 | −0.1715 | 1.9905 |
| FLJ12270 | 890555 \|\| [symbol] | −1.7535 | −1.1605 | 0.1535 | 0.8855 | −0.1385 | 0.7085 | 0.1485 | 0.5865 | 0.0985 | −0.8855 | 1.8355 | 1.8305 | −0.7475 |
| FLJ12844 | 893818 \|\| [symbol] | −0.4655 | −0.4255 | 0.7285 | 0.1795 | −0.0405 | −0.5405 | −0.3485 | −0.4895 | −0.4275 | −0.3865 | 1.1885 | 0.1235 | 0.3835 |
| FLJ12650 | 690008 \|\| [symbol] | 1.0735 | −1.1135 | −0.2965 | −0.7876 | 2.0425 | 0.6995 | 0.0505 | −0.0845 | 0.5515 | 0.3855 | 0.1655 | −0.5385 | −0.0065 |
| FLJ12684 | 685218 \|\| [symbol] | 0.9885 | 0.7895 | 0.7505 | 1.4115 | 2.0015 | 1.2325 | −0.1145 | 0.3676 | 0.4405 | 1.4025 | 0.0225 | 0.4116 | 0.9545 |
| FLJ12735 | 897973 \|\| [symbol] | −0.079 | 0.169 | −2.48 | 0.512 | −0.466 | 0.237 | 0.82 | −0.99 | −0.837 | −0.766 | −0.209 | −0.759 | −0.315 |
| FLJ12750 | 885380 \|\| [symbol] | −0.94 | −1.424 | −0.517 | −1.979 | −0.9 | −2.948 | −1.803 | −1.416 | 0.588 | −2.566 | −1.171 | −0.144 | −1.851 |
| FLJ12895 | 683723 \|\| [symbol] | −0.3805 | −0.5855 | 2.3085 | 0.1075 | −0.4665 | 2.6775 | −0.0005 | −0.3155 | 0.2005 | −0.6285 | −0.7115 | −1.1655 | 2.3455 |
| FLJ13710 | 693274 \|\| [symbol] | 0.2385 | 0.1955 | −0.1395 | −0.3805 | 0.4955 | 0.0335 | 1.4815 | −0.6745 | 0.2775 | 0.4725 | 0.3865 | 0.3715 | −0.2835 |
| FLJ13855 | 881768 \|\| [symbol] | −0.467 | −0.156 | 0.407 | 0.26 | −0.62 | −0.284 | −0.157 | −0.117 | −0.402 | −0.519 | −0.295 | −0.283 | −0.02 |
| FLJ13912 | 696739 \|\| [symbol] | −0.274 | −1.148 | 0.501 | −0.06 | 0.75 | 0.829 | −0.129 | 0.429 | 0.743 | −0.906 | 0.471 | 0.945 | −0.043 |
| FLJ14124 | 891127 \|\| [symbol] | −0.005 | −0.446 | −0.216 | 0.663 | 0.122 | 0.845 | 0.505 | −0.213 | 0.274 | −0.001 | 0.053 | 0.218 | −0.078 |
| FLJ14627 | 681398 \|\| [symbol] | −0.1055 | 2.0265 | 0.7145 | 0.4355 | −0.0555 | −0.0645 | 5.1925 | 0.0315 | −0.0885 | −0.1775 | 0.0145 | 0.0865 | 0.0295 |
| FLJ14666 | 686627 \|\| [symbol] | −0.515 | 0.117 | 0.494 | −0.279 | −1.245 | −0.625 | −0.545 | 0.427 | 0.594 | −0.598 | 0.428 | 1.081 | −0.498 |
| FLJ14904 | 887755 \|\| [symbol] | 0.042 | −1.23 | −0.423 | 0.155 | −0.239 | −1.481 | −2.484 | −1.371 | 1.901 | −1.311 | −1.394 | −0.586 | −2.18 |
| FLJ20035 | 895171 \|\| [symbol] | −0.775 | 0.168 | 1.057 | −0.049 | −0.347 | 0.543 | 0.638 | 0.119 | −0.223 | 0.954 | 1.87 | 1.301 | 0.269 |
| FLJ20152 | 594420 \|\| [symbol] | −0.198 | −0.448 | −0.561 | 0.132 | −0.121 | −0.013 | −0.063 | 0.013 | 0.157 | 0.267 | 0.379 | 0.425 | 0.565 |
| FLJ20171 | 695239 \|\| [symbol] | 0.76 | −0.046 | −0.149 | −0.97 | −0.608 | 0.759 | 0.869 | 0.237 | −0.989 | −0.238 | 0.158 | −0.289 | 0.818 |
| FLJ20244 | 684905 \|\| [symbol] | 0.026 | −0.019 | −0.282 | 1.071 | 0.554 | 0.545 | 0.703 | 0.051 | −0.215 | −0.608 | −1.404 | −0.231 | −0.283 |
| FLJ20273 | 683092 \|\| [symbol] | −0.393 | 0.105 | −0.199 | −0.203 | 0.62 | 0.205 | −0.013 | −0.265 | 0.014 | −0.087 | 1.237 | 1.079 | 0.098 |
| FLJ20315 | 689448 \|\| [symbol] | −0.083 | −0.771 | −0.258 | −0.095 | −0.134 | −0.244 | −0.169 | −0.897 | −0.156 | 1.024 | 0.674 | 0.837 | 0.244 |
| FLJ20323 | 897352 \|\| [symbol] | 0.0295 | 0.7265 | −0.3385 | −0.9995 | 0.2765 | 0.8805 | 0.5735 | −0.2445 | −0.1255 | 1.4305 | 0.6925 | 0.0035 | 1.0315 |
| FLJ20518 | 888202 \|\| [symbol] | −0.427 | −0.303 | −1.257 | −1.103 | −0.842 | −0.286 | −0.391 | −0.22 | −0.116 | −0.652 | −0.499 | −0.895 | −0.687 |
| FLJ20530 | 681453 \|\| [symbol] | 0.591 | −0.263 | 0.168 | −0.371 | 0.251 | 0.325 | 0.325 | 0.256 | −0.34 | 0.159 | −0.362 | −0.19 | 0.641 |
| FLJ20696 | 684792 \|\| [symbol] | −0.8795 | −0.8755 | 0.8155 | −0.0425 | 0.0475 | 0.4885 | −0.4275 | 0.7905 | −1.1425 | 0.6445 | 0.8105 | 1.0335 | 1.1905 |
| FLJ20718 | 692255 \|\| [symbol] | 0.258 | 0.219 | −0.292 | 0.055 | −0.273 | 0.206 | −0.533 | −0.153 | −0.031 | −0.094 | 0.873 | 1.752 | 0.765 |
| FLJ20772 | 688898 \|\| [symbol] | −0.2815 | −0.5555 | −0.0045 | −0.0755 | −0.1245 | 0.1805 | 0.2685 | −0.5775 | −0.2475 | −0.8015 | −0.8445 | −0.7975 | −0.2255 |
| FLJ20989 | 895210 \|\| [symbol] | 0.9205 | 0.1615 | −1.2595 | −1.0985 | −0.2535 | −0.5195 | 0.5955 | −0.5005 | 0.2025 | −0.1275 | −0.8295 | −0.5205 | −0.8765 |
| FLJ21019 | 889334 \|\| [symbol] | 0.5525 | −0.0935 | 0.4785 | 1.6365 | 1.1045 | 1.3095 | −0.4285 | 0.1765 | 0.7265 | −1.2565 | 0.1195 | 0.7525 | 0.0075 |
| FLJ21062 | 695900 \|\| [symbol] | −1.2415 | −0.3225 | −0.1205 | 0.1245 | 0.8325 | 0.2495 | 0.6705 | −2.2095 | 0.0385 | −0.4295 | −1.5455 | −2.2935 | −0.5235 |
| FLJ21159 | 688476 \|\| [symbol] | 0.1335 | −0.6335 | −1.5665 | 1.3215 | −0.2525 | −0.0375 | 0.6915 | −0.1576 | −0.6015 | 1.1075 | −0.2705 | 0.1735 | −0.1395 |
| FLJ21616 | 695280 \|\| [symbol] | −0.241 | 0.018 | −1.008 | 1.023 | −0.274 | 0.464 | 1.865 | 1.017 | −0.064 | 2.165 | 0.687 | 1.573 | 1.011 |
| FLJ21827 | 883572 \|\| [symbol] | −0.5085 | 0.7245 | −0.7565 | −0.3145 | −0.1285 | 0.6245 | −0.8745 | 0.5765 | 0.4975 | 0.8495 | 0.3965 | 0.2895 | 0.0715 |
| FLJ21963 | 888108 \|\| [symbol] | −0.521 | −0.509 | 0.051 | 0.122 | −0.237 | −0.213 | 0.119 | −0.673 | −0.513 | 0.297 | 0.216 | 0.598 | −0.091 |
| FLJ22104 | 890873 \|\| [symbol] | 0.7245 | 0.0295 | −0.5865 | −0.5355 | −0.6965 | 0.2285 | 0.8735 | −0.2905 | −0.2385 | 0.2685 | 0.3545 | 0.4795 | 0.7215 |
| FLJ22222 | 594558 \|\| [symbol] | −0.651 | 0.107 | 0.272 | 0.29 | −0.477 | −0.013 | −0.759 | 0.079 | 0.63 | −0.396 | −0.464 | −0.176 | 0.203 |
| FLJ22573 | 683583 \|\| [symbol] | 0.0395 | −0.2745 | −0.2115 | −1.1355 | 1.4505 | 2.0365 | 1.6045 | 0.3415 | 1.4655 | −0.0225 | −0.6275 | −0.6845 | −0.8515 |
| FLJ22794 | 892448 \|\| [symbol] | −0.612 | 1.013 | −0.39 | −0.032 | −0.05 | −0.038 | −0.183 | −0.091 | −0.359 | 0.347 | 0.005 | 0.089 | −0.29 |
| FLJ23188 | 892984 \|\| [symbol] | −0.5916 | 1.9035 | 1.3045 | 1.1335 | −0.0195 | 2.0905 | −0.1035 | −0.0855 | 0.4085 | 2.4485 | −0.0855 | 1.0455 | −0.3215 |
| FLJ23441 | 868418 \|\| [symbol] | −1.204 | −1.596 | −0.129 | 1.353 | 0.478 | −1.286 | −0.211 | −0.115 | −0.652 | −1.351 | 0.729 | 1.596 | 0.307 |
| FLJ25471 | 895557 \|\| [symbol] | −0.382 | −0.715 | −1.075 | −1.101 | −0.558 | −0.018 | 0.074 | −0.784 | −0.356 | −0.085 | −0.587 | −0.788 | −0.145 |
| FLJ31204 | 883983 \|\| [symbol] | −0.9455 | −0.0405 | 0.5375 | 0.0135 | 0.0225 | −0.3825 | 0.3545 | 0.0215 | 0.2285 | −0.4746 | 0.5085 | −0.4405 | 0.2795 |
| FLJ31795 | 688302 \|\| [symbol] | 0.0205 | −0.1665 | −0.0435 | −0.3895 | −0.3085 | −0.1825 | −0.3235 | −0.3685 | −0.4685 | −0.1555 | 0.0925 | 0.3785 | 0.7685 |
| FLJ32942 | 690591 \|\| [symbol] | 0.0815 | −1.3845 | −1.4875 | 0.6015 | 0.2975 | 0.2465 | −1.5155 | −0.4285 | 0.8435 | −1.9195 | −0.2155 | −0.2405 | −2.2155 |
| FLJ37970 | 897729 \|\| [symbol] | −0.515 | −1.794 | −2.303 | −2.778 | −2.233 | −1.067 | 0.674 | −1.198 | −0.393 | −3.219 | −0.848 | 0.894 | 0.493 |
| FLJ39370 | 894709 \|\| [symbol] | −0.6175 | 0.0775 | −0.7755 | 0.1025 | −0.0305 | −0.3975 | 0.3855 | −0.0085 | −0.5475 | −0.4435 | −0.2635 | 0.1585 | 0.0415 |
| FLJ90588 | 885870 \|\| [symbol] | 0.8545 | 1.5425 | 0.6515 | 0.1625 | −0.2145 | −0.8425 | 0.0485 | −0.1375 | −0.0925 | 1.4205 | 0.0735 | −0.1945 | −0.6245 |
| FN3KRP | 685326 \|\| [symbol] | −0.333 | −0.378 | −0.898 | 0.354 | 0.002 | 0.307 | 0.391 | −1.119 | 0.489 | −0.218 | 0.154 | 0.184 | 0.427 |
| FOLR1 | 697855 \|\| [symbol] | 0.0005 | 0.5185 | 0.6465 | 0.2275 | 0.9005 | −1.3195 | −1.4495 | −0.6495 | −0.9995 | −1.4695 | −0.7195 | −0.0005 | −1.6095 |
| FOXA1 | 692889 \|\| [symbol] | −0.3385 | 0.3195 | −0.1205 | 0.9985 | −0.2185 | 0.4875 | −0.1365 | 0.3895 | 0.6275 | −0.7945 | 0.1065 | 0.8345 | −0.0365 |
| FOXC1 | 772435 \|\| [symbol] | 0.271 | 0.904 | 0.059 | 1.454 | 0.728 | 2.004 | 1.73 | 0.458 | 0.469 | −0.844 | 1.387 | 0.804 | 0.448 |
| FOXP1 | 892043 \|\| [symbol] | 0.2095 | 0.3285 | −0.0755 | 0.7455 | −0.3905 | −0.2235 | 0.1035 | 0.4675 | −0.0925 | −0.4965 | −0.5285 | −0.4125 | −0.7275 |
| FREQ | 683314 \|\| [symbol] | 0.2025 | 0.7695 | −0.1035 | −0.1835 | 0.4515 | −0.4385 | −0.4195 | −0.7795 | 0.1315 | −0.4105 | −0.1395 | 0.2455 | −0.6005 |
| FTO | 893312 \|\| [symbol] | −0.0325 | 0.3075 | −0.6405 | 0.0325 | −0.7265 | −0.1685 | 0.7495 | −0.2465 | −0.5085 | 0.0316 | −0.6745 | −0.1625 | −0.6225 |
| FUBP1 | 690423 \|\| [symbol] | −0.7805 | −0.2355 | −0.7095 | −0.3235 | 0.9045 | −0.4455 | −0.2825 | 0.4805 | 0.7205 | −1.3585 | −0.8385 | −1.0465 | −0.0355 |
| FUCA1 | 691254 \|\| [symbol] | 0.021 | 0.63 | −0.318 | 0.433 | −1.073 | −0.545 | −0.48 | 0.284 | −0.363 | −0.441 | 0.258 | −0.26 | −1.158 |
| FUT8 | 695197 \|\| [symbol] | −0.3065 | 0.5145 | 0.2175 | 0.4915 | −0.8545 | 0.1785 | 0.3975 | −0.6445 | −0.4515 | 0.2065 | −0.7795 | −0.1495 | −0.2845 |
| FXYD6 | 887048 \|\| [symbol] | 0.841 | 0.617 | −0.294 | 0.09 | −0.896 | 1.264 | −0.451 | −0.19 | −0.293 | −1.388 | 0.428 | −1.132 | −0.766 |
| FYCO1 | 891750 \|\| [symbol] | 0.294 | 0.785 | −1.38 | −0.448 | −0.822 | 0 | −0.272 | 0.868 | −0.03 | −1.561 | −0.541 | −1.177 | −2.072 |
| FZD10 | 686248 \|\| [symbol] | −0.4155 | 0.3385 | 0.4545 | 0.3505 | −0.0345 | −0.1625 | 0.7895 | 0.2445 | −0.5585 | 0.1575 | −0.3545 | 0.2645 | −0.2855 |
| FZD7 | 691443 \|\| [symbol] | −1.3965 | −0.0985 | 3.0495 | 2.7575 | −0.0205 | −0.8455 | −0.8125 | 2.4325 | 3.3105 | −1.0305 | 1.3846 | 1.9755 | 0.0145 |
| FZD9 | 691926 \|\| [symbol] | 0.5475 | −0.0755 | 2.6905 | 0.7275 | −0.1085 | −1.0435 | −0.5545 | 1.2875 | 1.7735 | −1.0245 | 1.5045 | 1.7545 | −0.8785 |
| G1P2 | 894822 \|\| [symbol] | −0.7785 | −0.0195 | −0.1485 | −0.4345 | −0.3345 | −0.1945 | −0.9095 | 0.2455 | 0.2845 | −0.1265 | 0.2125 | −0.5535 | −0.3245 |
| G1P3 | 893731 \|\| [symbol] | 0.263 | 0.253 | 0.229 | −0.262 | −0.066 | −0.291 | −0.563 | −0.13 | −0.244 | −0.489 | −0.797 | −1.112 | −0.989 |
| GA17 | 093445 \|\| [symbol] | −0.7955 | −0.5895 | −0.0245 | 0.4785 | −0.0345 | 0.8395 | 0.4595 | −0.8175 | −0.8135 | 0.8685 | 1.2585 | 1.2545 | 0.7175 |
| GABARAP | 683557 \|\| [symbol] | 1.4945 | 0.5355 | −0.5355 | 1.5685 | 0.3785 | 0.4975 | 1.8285 | 0.2185 | 0.8085 | 1.2255 | 0.5765 | 1.2365 | 0.2565 |
| GAJ | 696722 \|\| [symbol] | 2.6505 | 2.8795 | 0.7425 | 2.7345 | 3.3665 | 1.7195 | 2.3125 | 0.8115 | −0.7745 | −0.9175 | 2.8145 | 1.1365 | 3.7625 |
| GALE | 681740 \|\| [symbol] | −0.103 | 0.222 | 0.778 | 0.373 | −0.549 | 0.615 | 0.379 | −0.043 | 0.286 | −0.263 | 0.484 | 0.87 | −0.076 |
| GALNT14 | 893378 \|\| [symbol] | 0.3485 | 0.6135 | 1.4215 | 2.1576 | 0.6605 | 1.0145 | −0.0535 | 0.7515 | 1.0015 | −2.0585 | −0.4355 | −0.7195 | 0.1135 |
| GALNT2 | 892425 \|\| [symbol] | −1.4405 | −1.3875 | 0.2545 | 1.6345 | −0.6805 | −2.4295 | −1.4585 | −0.7915 | −0.8045 | −1.6585 | −0.7365 | −0.6845 | −1.6345 |
| GALNT7 | 684972 \|\| [symbol] | −0.364 | 0.596 | 0.341 | −0.077 | 0.094 | −0.469 | −0.404 | 0.567 | 0.029 | −0.248 | 0.329 | 0.765 | −0.006 |
| GAMT | 887858 \|\| [symbol] | −0.257 | 0.27 | 0.45 | 0.516 | 0.071 | 1.152 | 0.458 | 0.971 | 1.447 | 1.458 | 1.187 | 1.285 | 0.774 |
| GAPD | 695932 \|\| [symbol] | −0.006 | 0.508 | −0.575 | −0.596 | 0.218 | 0.292 | 0.784 | −0.148 | 0.357 | 1.553 | −0.317 | −0.775 | 0.699 |
| GARS | 693904 \|\| [symbol] | −1.818 | −2.068 | −0.501 | −0.845 | −0.235 | 0.125 | −1.562 | −0.882 | 0.685 | −2.62 | −0.77 | 0.454 | −0.494 |
| GART | 683079 \|\| [symbol] | 0.7865 | −0.1815 | 0.4285 | 1.4615 | −1.0635 | −1.7285 | 0.3395 | 0.7075 | −0.3845 | 2.1145 | −0.3935 | −0.3165 | −1.4805 |
| GATA3 | 896046 \|\| [symbol] | 0.3895 | −0.1995 | 0.0815 | 0.4585 | 0.3585 | 0.0785 | 0.7635 | 0.0535 | 0.0575 | −0.5235 | 0.7675 | 0.4475 | 0.4185 |
| GATM | 885950 \|\| [symbol] | −0.548 | −0.381 | −0.569 | −1.28 | −1.729 | −0.531 | −1.23 | −0.008 | −0.106 | 0.978 | −1.017 | −0.765 | −0.319 |
| GBA | 591089 \|\| [symbol] | 0.128 | 0.418 | −0.438 | 0.286 | −0.28 | 0.301 | 0.26 | 0.117 | −0.21 | 0.593 | 0.448 | 0.134 | 0.126 |
| GDAP1 | 689715 \|\| [symbol] | 0.213 | 0.145 | 0.232 | 0.576 | −0.164 | 0.233 | 0.016 | 0.65 | 1.339 | 0.55 | 0.073 | 0.18 | −0.174 |
| GDI2 | 588226 \|\| [symbol] | −0.1865 | −0.9785 | −1.2875 | −0.3205 | −0.8345 | −0.3635 | 0.3935 | −0.2875 | 0.6705 | −0.3355 | −0.4545 | −0.0605 | 0.2175 |
| GGA1 | 683921 \|\| [symbol] | −0.6995 | 0.5815 | −0.1915 | 0.1155 | 0.0005 | 0.8445 | 0.3545 | −0.1625 | 0.5705 | −1.2485 | −0.9155 | −0.1625 | −0.4395 |
| GKAP1 | 862770 \|\| [symbol] | 0.5125 | 0.7785 | −0.1205 | 0.4845 | 0.2245 | 0.5945 | −0.2145 | 0.5625 | 0.3955 | −0.3155 | −0.1745 | −0.3745 | −0.1525 |
| GLCCI1 | 689758 \|\| [symbol] | −0.0025 | 0.4095 | −0.7305 | 0.8295 | −1.3345 | −0.5535 | −0.9525 | −0.0415 | 0.4375 | −0.6625 | −0.3785 | −0.2815 | 0.2875 |
| GLG1 | 681938 \|\| [symbol] | −0.404 | 0.552 | 0.309 | −1.298 | 0.104 | 0.197 | 0.421 | 0.8 | 0.163 | 1.794 | 0.337 | −0.333 | 0.429 |
| GLTSCR2 | 686785 \|\| [symbol] | −0.286 | 0.812 | 1.446 | −1.155 | −1.17 | −1.048 | −0.2 | 0.156 | 0.485 | −1.528 | −0.182 | 0.442 | 0.123 |
| GNB4 | 897071 \|\| [symbol] | 0.061 | −0.308 | 2.29 | 0.286 | −0.351 | 0.063 | −0.001 | 0.078 | 0.001 | −0.077 | −0.236 | 0.053 | 0.444 |
| GOLPH2 | 089832 \|\| [symbol] | 0.8845 | 0.5505 | 0.0385 | 0.6475 | 0.3275 | 1.5155 | 2.2925 | 0.6145 | 0.2955 | −0.5185 | 0.9205 | 0.2815 | 2.3895 |
| GPATC1 | 687697 \|\| [symbol] | −0.0515 | 1.0565 | 0.0595 | −0.8145 | 0.0545 | 0.6355 | −0.1765 | 0.4825 | 0.0845 | −1.9405 | −0.8295 | −1.7545 | −0.8945 |
| GPC2 | 692352 \|\| [symbol] | −0.1575 | 0.5545 | 1.0635 | 0.2295 | −0.1615 | 0.2455 | 1.2635 | 0.2885 | 0.8465 | −0.5825 | −0.0815 | 0.2845 | 2.5855 |
| GPR125 | 894724 \|\| [symbol] | 0.274 | 0.814 | 1.734 | 0.484 | 0.99 | 0.304 | −0.168 | 0.318 | 1.474 | −0.555 | −0.046 | 0.019 | 0.315 |
| GPR160 | 882327 \|\| [symbol] | −0.0045 | 0.2095 | −0.0515 | 1.1245 | −0.0695 | 3.1685 | 1.5525 | 0.7045 | 0.2805 | −1.1475 | −0.1645 | 0.6225 | 2.6135 |
| GPR56 | 688865 \|\| [symbol] | −0.1725 | 0.0965 | −0.0185 | −0.4845 | −0.4345 | 0.1995 | 0.6025 | 0.2175 | 0.1755 | −1.6285 | 0.4235 | 0.1345 | −0.5485 |
| GPRC5C | 696709 \|\| [symbol] | −0.3645 | 0.6205 | −0.6625 | −0.7455 | −0.6855 | 0.2915 | 1.2236 | −0.0175 | −0.1905 | 0.3645 | −0.0465 | −0.7835 | 0.5815 |
| GPSM1 | 582325 \|\| [symbol] | −0.192 | −0.74 | −0.638 | −0.017 | −0.748 | −0.952 | −0.37 | −0.541 | 0.581 | 0.835 | −0.3 | −0.247 | −0.523 |
| GPSM2 | 690902 \|\| [symbol] | 1.122 | 0.861 | 0.702 | −0.412 | −0.182 | 0.923 | 1.598 | −0.812 | −0.36 | 2.504 | 0.853 | 0.673 | 0.855 |
| GPT | 693608 \|\| [symbol] | −0.8305 | 1.4965 | −0.5025 | −1.1885 | −0.8385 | 1.1185 | 0.0765 | 1.6475 | 0.5885 | 0.0465 | 0.2715 | −1.0535 | −0.8215 |
| GPT2 | 689304 \|\| [symbol] | −0.241 | 0.355 | −1.603 | 3.521 | −0.67 | −0.437 | 2.191 | 1.743 | 1.458 | −1.13 | 1.88 | 2.115 | −1.931 |
| GPX7 | 694626 \|\| [symbol] | 1.15 | 2.607 | 2.858 | 2.303 | 2.18 | 2.734 | 0.006 | 1.95 | 2.164 | 0.67 | 0.923 | 1.112 | 2.326 |
| GRB14 | 691136 \|\| [symbol] | 0.7105 | 0.8275 | 0.1355 | 0.7695 | −1.1605 | −0.6165 | −0.0155 | 0.5735 | 0.1995 | 0.3225 | −0.3935 | 0.7325 | −1.1365 |
| GRB7 | 691667 \|\| [symbol] | 1.7495 | 1.9905 | 2.0995 | 1.5095 | −0.1385 | 2.4525 | −0.1425 | 3.7255 | −0.4995 | 0.0405 | 0.8235 | 1.3855 | 2.0195 |
| GSC | 684798 \|\| [symbol] | 0.376 | 0.497 | −0.108 | −1.021 | 0.06 | 0.636 | 0.036 | 0.155 | 0.467 | 0.408 | 0.043 | 0.231 | −0.282 |
| GSDML | 084490 \|\| [symbol] | 0.048 | 0.057 | 0.031 | 0.098 | −0.157 | 0.205 | −0.592 | −0.058 | −0.253 | 0.154 | −0.305 | 0.27 | 0.331 |
| GSK3B | 882653 \|\| [symbol] | 1.121 | −0.527 | −0.437 | −0.186 | −0.643 | −0.374 | 0.389 | −0.662 | −0.418 | 0.006 | −0.706 | −0.055 | −0.734 |
| GSTA1 | 890111 \|\| [symbol] | 0.8975 | −0.3075 | −2.3485 | 0.7815 | 2.2325 | −0.8485 | −2.9985 | 0.1025 | −0.6025 | 0.6445 | 0.8275 | 1.0695 | 1.2955 |
| GSTK1 | 691113 \|\| [symbol] | 1.083 | 0.224 | −1.916 | −1.387 | −0.883 | 0.928 | 0.455 | −0.512 | −1.204 | −1.594 | 0.891 | 0.017 | 0.819 |
| GSTM3 | 686813 \|\| [symbol] | 0.2485 | 0.2175 | −0.2895 | 1.4785 | 0.8385 | −2.2375 | −1.4625 | −0.2145 | −1.9945 | −2.0195 | −0.1845 | 0.5325 | −1.7025 |
| GSTP1 | 690952 \|\| [symbol] | −0.934 | 0.467 | 0.594 | −1.223 | −0.947 | −0.37 | −0.654 | 0.999 | 0.295 | −1.059 | 0.148 | −0.453 | −0.32 |
| GSTT1 | 684076 \|\| [symbol] | 0.2255 | 0.2875 | −0.1375 | −0.4695 | 0.9085 | 0.4355 | −0.5345 | −0.3055 | −0.3835 | 0.3275 | −0.0845 | −0.2445 | 0.3105 |
| GSTT2 | 885053 \|\| [symbol] | −0.3725 | −0.1935 | −0.6785 | −0.4595 | 0.2725 | −0.1925 | 0.9475 | −0.2355 | −0.0995 | −1.1935 | −0.0595 | −0.7405 | −0.3305 |
| GTF2F2 | 697821 \|\| [symbol] | −0.067 | 0.34 | −0.411 | −0.938 | 0.64 | 0.261 | −0.382 | −0.07 | 0.005 | 0.33 | −0.061 | −0.572 | 0.024 |
| GTF2I | 692303 \|\| [symbol] | 0.2785 | −0.4145 | −0.3495 | 0.1105 | −0.0455 | 0.3715 | −0.0165 | −0.0255 | −0.0625 | 0.3735 | 0.1675 | 0.6185 | 0.7205 |
| GTF3A | 693886 \|\| [symbol] | −0.123 | −0.531 | 0.411 | −0.299 | −0.013 | 0.838 | 0.502 | −0.035 | −0.191 | 1.548 | 0.587 | 0.472 | 0.947 |
| GTPBP3 | 682310 \|\| [symbol] | −1.425 | 0.651 | 0.676 | 1.228 | −1.438 | −1.219 | 0.547 | 0.877 | 0.824 | −0.826 | 0.053 | 1.297 | −0.567 |
| GTPBP4 | 689649 \|\| [symbol] | −0.833 | 0.352 | 0.195 | 0.131 | 0.09 | 0.75 | 1.166 | 0.265 | −0.318 | −0.424 | 0.156 | −0.214 | 0.728 |
| H1F0 | 692476 \|\| [symbol] | −0.5015 | 0.1155 | 0.6305 | −0.0325 | 0.8035 | 0.2115 | −0.3675 | 0.4355 | 0.0725 | −0.0595 | 0.2695 | 0.4835 | 0.4005 |
| H2AFV | 695317 \|\| [symbol] | −0.2655 | −0.4325 | 0.3275 | 0.4805 | −0.5185 | 0.5615 | 0.3255 | 0.4775 | −0.8435 | 0.7585 | 0.4165 | 0.0935 | −0.1985 |
| H2AFY | 896406 \|\| [symbol] | 0.2725 | 0.6015 | −0.1305 | 1.8735 | 1.5515 | 0.7905 | 1.2085 | 0.4755 | 1.9585 | −0.8175 | −0.6935 | −0.7775 | −0.2775 |
| H2AFZ | 683032 \|\| [symbol] | −1.1405 | 0.2505 | 0.0125 | −1.0075 | −0.2325 | 0.2265 | −0.1865 | 0.0135 | −0.9145 | −1.1005 | −0.3675 | −0.8115 | −0.4325 |
| H2BFS | 685337 \|\| [symbol] | −0.3415 | 0.3895 | −0.2925 | −0.8255 | −0.6745 | −0.0615 | 0.0965 | −0.3475 | 0.4695 | 0.7765 | 0.4315 | −0.0265 | 0.1845 |
| H3F3B | 690295 \|\| [symbol] | −0.047 | −0.183 | 0.135 | −0.363 | −0.702 | 0.124 | 0.714 | −0.413 | −0.014 | 0.61 | −0.438 | 0.291 | −0.213 |
| H41 | 682811 \|\| [symbol] | −0.0475 | 0.1965 | 0.3215 | −0.0995 | 0.1015 | −0.1275 | 0.2955 | 0.8055 | −0.2345 | −0.2865 | −0.3205 | −0.1375 | −0.6075 |
| HAGH | 696380 \|\| [symbol] | 0.177 | 0.157 | −0.053 | 0.645 | −0.736 | −0.397 | 0.238 | −0.009 | −0.366 | −0.369 | 0.316 | 0.007 | 0.1 |
| HBG1 | 688388 \|\| [symbol] | −0.5635 | 0.2405 | 0.3845 | −0.1595 | −0.4695 | 0.8555 | 0.7415 | 1.4065 | 0.0855 | 1.3305 | 1.1245 | 0.8395 | 1.1805 |
| HBL01 | 697677 \|\| [symbol] | 0.227 | 0.568 | 0.811 | −0.932 | −1.236 | −0.6 | −0.21 | 0.117 | 0.511 | −0.262 | 1.114 | 1.405 | 0.114 |
| HCAP-G | 691887 \|\| [symbol] | 0.4605 | 0.7915 | −0.7425 | −0.8035 | 1.0675 | 0.9535 | 0.5565 | 0.5245 | 0.8185 | 0.1685 | 0.6675 | −0.0335 | 2.2845 |
| HCP5 | 695382 \|\| [symbol] | 0.856 | 0.156 | −0.493 | −0.857 | −0.522 | −0.198 | −0.081 | −0.149 | −0.162 | 0.012 | −0.038 | −0.549 | −0.079 |
| HDAC2 | 697848 \|\| [symbol] | −0.175 | −0.02 | −0.135 | −0.597 | −0.911 | 0.384 | 0.041 | −0.075 | −0.2 | −0.488 | −0.915 | −0.815 | −0.721 |
| HDC | 688061 \|\| [symbol] | −0.0095 | −0.1585 | 0.9645 | 0.2555 | 0.0615 | −0.0875 | −0.2735 | −0.0075 | −0.1455 | −0.1355 | 0.1965 | 0.5855 | 0.5055 |
| HEBP1 | 697035 \|\| [symbol] | 1.1 | −0.989 | −0.251 | 0.654 | 0.521 | −0.026 | 1.121 | −1.771 | 0.107 | −0.479 | −0.274 | 1.102 | 0.149 |
| HIPK2 | 691802 \|\| [symbol] | −0.243 | −0.687 | −0.822 | −0.307 | −0.305 | −0.11 | −0.366 | −0.557 | −0.37 | −0.036 | −0.702 | −0.508 | −0.483 |
| HIPK2 | 694742 \|\| [symbol] | 0.3475 | −0.1135 | 0.7955 | 2.2905 | 2.2295 | 1.1325 | 2.0965 | 1.0385 | 2.0965 | −1.2435 | −1.3505 | −1.0085 | −0.8145 |
| HIS1 | 694010 \|\| [symbol] | 0.2145 | 0.3015 | −0.1375 | 1.6125 | 1.7345 | 0.6315 | 1.5155 | 0.6015 | 1.8285 | −0.7215 | −1.0625 | −1.0035 | −0.5055 |
| HIST1H2A | 884611 \|\| [symbol] | 0.285 | 0.241 | −0.068 | 1.732 | 1.441 | 0.596 | 1.838 | 0.705 | 1.925 | −0.83 | −1.203 | −1.202 | −0.465 |
| HIST1H2B | 697885 \|\| [symbol] | 0.1215 | 0.1555 | 0.0705 | 1.6495 | 1.4055 | 0.4395 | 1.5135 | 0.8295 | 1.7615 | −1.0315 | −1.0155 | −1.3175 | −0.4135 |
| HIST1H2B | 681566 \|\| [symbol] | 0.351 | 0.348 | −0.081 | 1.551 | 1.559 | 0.594 | 1.483 | 0.88 | 1.961 | −0.901 | −1.183 | −1.059 | −0.588 |
| HIST1H28 | 695646 \|\| [symbol] | −0.8155 | 0.4935 | −0.2815 | −0.8325 | 0.2105 | 0.1485 | −0.8085 | 0.0295 | −0.8445 | −0.8725 | −0.5215 | −0.4845 | −0.7455 |
| HIST1H2B | 683833 \|\| [symbol] | 0.3055 | −0.4585 | 0.4975 | 1.0095 | 0.1525 | 0.3216 | 0.7885 | 0.2275 | 0.5905 | 0.9875 | 0.8015 | 0.7785 | 0.6785 |
| HIST1H3C | 693912 \|\| [symbol] | −0.0385 | 0.5265 | −0.2245 | 0.0575 | 0.2015 | 1.1655 | 0.9235 | 0.4015 | −0.2915 | 1.1505 | 0.0155 | 0.2895 | 0.9525 |
| HM13 | 695097 \|\| [symbol] | −1.0405 | 0.3725 | 1.0875 | 0.7755 | 1.1365 | 1.5135 | 0.8355 | 0.9835 | −0.4575 | 0.5985 | 1.3765 | 0.6595 | 3.7065 |
| HMGB3 | 690848 \|\| [symbol] | −0.7315 | 0.2045 | 0.0035 | −0.3225 | 0.2255 | 0.2915 | 0.0675 | −0.0355 | 0.0875 | −0.3965 | 0.4825 | −0.1875 | 0.5725 |
| HN1 | 689578 \|\| [symbol] | −0.764 | −0.115 | 0.487 | −0.75 | −0.758 | −0.841 | −0.985 | 0.12 | −0.325 | −0.579 | 0.277 | 0.534 | −0.676 |
| HNRPA3P | 687868 \|\| [symbol] | 1.0505 | 0.5365 | −0.0875 | −0.6775 | −0.8235 | 1.5115 | −0.5585 | −0.0325 | −0.0795 | 0.5875 | −0.2085 | −0.6565 | −0.3485 |
| HNRPDL | 689713 \|\| [symbol] | 1.45 | 0.401 | −1.5 | −0.674 | −2.93 | −0.579 | −0.319 | 0.048 | −0.308 | −0.283 | −0.804 | −1.573 | −0.648 |
| HOXA5 | 682213 \|\| [symbol] | 0.3945 | 0.5205 | −2.0465 | 0.5995 | −1.2745 | 2.9585 | 1.1485 | 0.7505 | 0.9225 | 0.4445 | −0.0715 | 0.9085 | −1.6205 |
| HOXA7 | 693224 \|\| [symbol] | −0.6055 | 0.1185 | 0.7145 | 0.0575 | 0.0795 | 4.0675 | 0.3525 | 0.3775 | 0.5895 | −0.7295 | 0.1945 | 0.7555 | −0.3535 |
| HOXB2 | 695779 \|\| [symbol] | 0.4935 | −0.2565 | −0.6115 | 0.5955 | −0.1975 | 4.3155 | −0.7505 | 0.4885 | 0.9625 | −0.5235 | 0.0095 | 0.7595 | −0.7945 |
| HOXB6 | 698926 \|\| [symbol] | 0.5545 | −0.0005 | 2.2125 | 2.5515 | 1.2015 | 0.7075 | 0.0375 | 0.2305 | 0.5195 | −0.0015 | 1.6395 | 2.1535 | 3.1405 |
| HOXB7 | 687026 \|\| [symbol] | −0.2545 | 0.0525 | 0.7105 | 0.4605 | 1.5335 | 0.7825 | 0.8385 | 0.7655 | 1.3715 | −0.0795 | 1.1065 | 1.3295 | 0.2945 |
| HOXC10 | 688125 \|\| [symbol] | −0.431 | −0.243 | 0.515 | 1.179 | 1.389 | 0.874 | −0.878 | 0.511 | 0.157 | −0.821 | 0.054 | 0.689 | 1.725 |
| HOXC13 | 888022 \|\| [symbol] | −0.5565 | −0.5185 | −0.7215 | −0.1975 | −1.4625 | −1.2795 | −0.1025 | −1.5305 | 0.3015 | 0.4955 | −0.4245 | −0.5525 | −0.2685 |
| HOXC9 | 886974 \|\| [symbol] | −0.193 | 0.034 | −0.694 | −0.658 | −0.315 | 0.671 | −0.257 | −0.409 | −0.649 | 0.146 | −0.555 | −0.284 | −0.842 |
| HOXD3 | 888881 \|\| [symbol] | −0.7255 | 0.3285 | −0.8215 | −8.4915 | 0.0925 | −0.1165 | 0.1745 | −0.1345 | 0.4685 | −0.3545 | −0.3265 | −0.5815 | −0.5145 |
| HPCL2 | 693166 \|\| [symbol] | 0.086 | 1.059 | −0.141 | 1.41 | −0.382 | 2.558 | 1.655 | 0.366 | 0.955 | 3.24 | 1.44 | 0.801 | −0.297 |
| HPS3 | 684956 \|\| [symbol] | 1.0185 | 0.5705 | −0.3435 | 0.2355 | 0.9925 | −0.2865 | 1.2055 | 0.3785 | 0.2095 | 0.7635 | 0.4625 | 0.2315 | −0.1385 |
| HRASLS | 689305 \|\| [symbol] | −0.1735 | −0.3255 | −0.7835 | −0.7425 | −0.4905 | 0.5565 | 0.0305 | −0.0305 | −0.7255 | 1.3705 | 0.2735 | 0.7635 | 1.0085 |
| HRB | 692071 \|\| [symbol] | 3.956 | 1.43 | 1.507 | 1.214 | 2.313 | −0.312 | 1.631 | 0.146 | 0.345 | 2.123 | 1.055 | 2.184 | 2.208 |
| HRSP12 | 681060 \|\| [symbol] | −0.063 | 0.914 | −0.813 | 0.015 | −0.188 | 0.486 | −0.307 | 1.23 | −0.328 | 0.909 | 1.209 | 1.154 | 0.579 |
| HSD17B2 | 892999 \|\| [symbol] | 0.4035 | −0.0285 | −0.0005 | 0.0425 | −0.1145 | 0.7605 | 0.8125 | 0.0025 | 0.1076 | 0.8445 | −0.0515 | 0.0005 | 0.4825 |
| HSF1 | 894939 \|\| [symbol] | 0.2855 | −0.7595 | 1.3035 | 1.5075 | 0.9475 | −0.9395 | 1.1515 | 0.0805 | 2.5425 | −1.5385 | −1.2435 | −0.4465 | −1.3335 |
| HSPA14 | 694810 \|\| [symbol] | 1.047 | 0.596 | 1.078 | 1.67 | 1.363 | 1.52 | 1.145 | 0.956 | 1.122 | −0.122 | −0.03 | 0.156 | 1.947 |
| HSPA2 | 696880 \|\| [symbol] | −0.3265 | 0.0935 | 0.3515 | −0.0835 | −0.4115 | 0.1575 | 0.8625 | −0.5625 | −0.2325 | 0.4495 | −0.2465 | 0.0455 | −0.2815 |
| HSPA5BP [symbol] | 893776 \|\| [symbol] | −0.361 | 0.826 | −0.197 | −0.168 | −0.412 | −0.177 | −1.059 | −0.022 | 0.333 | 0.393 | 0.779 | 0.025 | −0.453 |
| HSPC016 | 685206 \|\| [symbol] | −0.271 | 0.086 | −0.264 | 0.441 | −0.649 | 0.214 | −1.113 | 0.236 | 0.065 | 0.333 | −0.083 | −0.22 | −0.128 |
| HSPC138 | 696635 \|\| [symbol] | 0.104 | −1.226 | −0.059 | −0.656 | 0.422 | 0.974 | 0.815 | −0.087 | 0.408 | −0.458 | 0.868 | 0.403 | 0.402 |
| HSPC148 | 687877 \|\| [symbol] | −0.4145 | −0.2695 | 1.1105 | −0.3935 | 0.0545 | 1.3315 | 1.5095 | 0.2905 | 0.9365 | −0.1915 | 1.4765 | 0.8576 | 0.3105 |
| HSPC150 | 892010 \|\| [symbol] | −0.0335 | −0.3826 | −0.2275 | 0.0105 | −0.5085 | 0.3435 | 0.7395 | −0.6975 | −0.0965 | 0.7065 | −1.0135 | −0.9545 | −0.1625 |
| HSPC163 | 891987 \|\| [symbol] | −0.161 | −0.413 | 0.798 | −1.325 | −0.71 | −0.754 | −0.935 | −0.21 | −0.891 | −0.016 | 0.131 | −0.874 | −0.921 |
| HSPC266 | 693184 \|\| [symbol] | −0.067 | 0.287 | −0.232 | −1.433 | 0.307 | 0.206 | 0.149 | −0.22 | −0.523 | 0.708 | 0.53 | 0.835 | −1.035 |
| HSU79274 | 693843 \|\| [symbol] | 0.154 | 0.755 | 0.628 | 0.347 | −0.188 | 0.227 | 1.084 | −0.061 | −0.168 | 0.529 | 0.574 | 0.516 | 0.78 |
| HTATIP2 | 593718 \|\| [symbol] | −0.721 | −0.908 | 0.259 | 0.541 | 0.469 | 0.577 | 0.425 | −0.124 | −0.848 | 0.83 | 0.202 | 0.061 | 2.822 |
| ICMT | 681046 \|\| [symbol] | 0.618 | 1.238 | −0.312 | 0.205 | −2.053 | −1.232 | −0.248 | −0.159 | −0.827 | 0.761 | −0.41 | −1.402 | −1.874 |
| ICT1 | 683297 \|\| [symbol] | 0.849 | 1.117 | 0.318 | 1 | 1.774 | 0.816 | 1.426 | 0.537 | 0.223 | 0.904 | 0.963 | 0.882 | 1.301 |
| ID4 | 685578 \|\| [symbol] | −0.1035 | 0.4815 | 0.5695 | −0.3845 | −0.2995 | 0.5665 | 0.6565 | −0.1745 | 0.1925 | −0.3765 | −0.6675 | −0.4185 | −1.1385 |
| IDH2 | 681706 \|\| [symbol] | −0.919 | 0.368 | 1.189 | 0.422 | 0.079 | 0.506 | −0.015 | 1.024 | −0.428 | 0.734 | 1.018 | 0.923 | 0.751 |
| IDS | 696420 \|\| [symbol] | −0.3295 | 0.1165 | 1.5775 | 1.2285 | −0.7755 | −0.8355 | −1.1525 | 1.9455 | 1.6355 | −0.9615 | 0.4075 | 1.5655 | −0.2725 |
| IFI30 | 695270 \|\| [symbol] | −0.4495 | 0.1615 | 0.8685 | 0.1875 | −0.9615 | −0.4995 | −0.5015 | 0.6465 | 1.3875 | −0.7615 | 0.2695 | 0.5095 | −0.4845 |
| IFI44 | 881212 \|\| [symbol] | −0.0145 | −0.3825 | 1.7325 | 0.0095 | −1.1655 | −1.0755 | −1.4015 | 1.0245 | 1.1485 | −1.1855 | 0.3715 | 0.7285 | −0.5295 |
| IFIT5 | 884503 \|\| [symbol] | −0.1845 | −0.0015 | −0.7755 | 0.5085 | −0.0795 | 0.2305 | 0.0575 | 0.1975 | 0.1815 | −0.0415 | −0.2605 | −0.3105 | −0.8305 |
| IFITM1 | 990866 \|\| [symbol] | −0.1295 | −0.4755 | −0.8485 | −0.2585 | 0.0915 | −0.3855 | 0.2035 | −0.2245 | −0.3715 | 0.2585 | −0.2755 | 0.3325 | −0.4865 |
| IGBP1 | 697847 \|\| [symbol] | −1.1085 | −0.3715 | 0.2285 | 0.7035 | 1.2755 | −0.6445 | −1.6905 | −0.0795 | 0.9975 | −3.2035 | 0.4255 | 1.0745 | 0.1955 |
| IGFALS | 692644 \|\| [symbol] | 0.2735 | −0.0835 | −0.1035 | −0.5025 | 1.0345 | 0.1285 | −0.4395 | −0.3935 | −0.2325 | 0.6605 | −0.2215 | 0.6535 | −0.0005 |
| IGPBP2 | 689980 \|\| [symbol] | 0.4005 | 0.0865 | −0.0105 | −0.6395 | 0.4216 | 0.0575 | −0.3465 | −0.3055 | −0.3035 | 0.7705 | −0.1175 | 0.7745 | −0.2005 |
| IGHG1 | 882030 \|\| [symbol] | 0.2385 | 1.0085 | −2.0475 | −2.4545 | −1.8445 | −0.5605 | 0.9185 | −2.4315 | −1.4565 | −0.8625 | −0.9345 | −0.5895 | 0.3885 |
| IGHG1 | 696457 \|\| [symbol] | −0.791 | 0.101 | −1.085 | −1.297 | −0.801 | 0.178 | 1.137 | −1.188 | −0.724 | 1.229 | −0.268 | 0.951 | −0.203 |
| IGJ | 687674 \|\| [symbol] | −0.829 | 0.14 | −2.258 | 0.134 | 1.153 | −0.268 | 1.087 | −1.199 | −1.773 | −0.452 | −0.629 | 0.983 | −0.7 |
| IGKC | 690921 \|\| [symbol] | −0.139 | 0.371 | −0.253 | 0.382 | −0.301 | 0.078 | −0.211 | −0.812 | −0.525 | 0.644 | −0.393 | 1.045 | −0.38 |
| IGKC | 691031 \|\| [symbol] | −0.957 | −0.034 | −1.581 | 0.092 | 0.779 | −0.562 | 0.196 | −0.807 | −0.748 | 0.041 | −0.261 | 1.806 | −1.002 |
| IGLC2 | 882716 \|\| [symbol] | 0.8155 | 0.6445 | 0.4925 | 0.6185 | −0.1645 | 0.4265 | 0.6475 | 0.2975 | 0.1685 | −0.1825 | 1.5175 | 1.4605 | 0.2855 |
| IGLL1 | 691836 \|\| [symbol] | 1.3175 | −0.1425 | 0.0525 | −0.3055 | −0.5975 | −0.2225 | −0.2555 | −0.3125 | −0.3405 | −0.3985 | 0.2965 | −0.6985 | −0.1445 |
| IL13RA1 | 697838 \|\| [symbol] | 0.814 | −0.349 | −0.286 | 0.589 | 0.245 | −0.167 | 0.393 | −0.067 | 0.06 | −0.133 | 0.017 | −0.033 | −0.134 |
| IL17B | 685612 \|\| [symbol] | 0.0395 | −1.1795 | −0.4735 | −1.3675 | −1.4635 | −1.2715 | −2.2415 | −1.6725 | −0.0145 | −1.1195 | −1.1215 | −0.4665 | −1.7185 |
| IL23A | 690046 \|\| [symbol] | −0.1095 | 0.2495 | 0.0425 | −0.0975 | 0.4976 | 0.0625 | 0.8155 | 0.1585 | 0.0415 | −0.5955 | 0.3945 | 0.2495 | −0.0955 |
| IL6ST | 690686 \|\| [symbol] | 0.5775 | 0.2385 | −0.5855 | −0.8035 | −0.4525 | −0.4775 | 0.9825 | −0.4245 | −0.6105 | 0.8505 | 1.3655 | 0.8415 | 0.4985 |
| ILF2 | 681204 \|\| [symbol] | −0.055 | −0.232 | −0.131 | −0.008 | 0.233 | 0.11 | 0.008 | −0.342 | −0.868 | −0.358 | −0.302 | 0.072 | −0.012 |
| IMPA2 | 890416 \|\| [symbol] | 1.1075 | 1.0415 | −1.2335 | −1.0975 | 0.3795 | −0.7835 | 1.0365 | 0.7995 | −1.2975 | 1.6185 | 1.4355 | 0.2465 | −1.5755 |
| ING1L | 681976 \|\| [symbol] | 0.598 | −0.574 | −0.481 | −0.035 | 0.575 | 0.425 | 0.22 | −0.457 | −0.191 | −1.251 | −0.183 | 0.443 | 0.242 |
| INHBB | 695995 \|\| [symbol] | −0.3405 | −0.2575 | 1.9825 | 0.2045 | −0.3985 | 1.1165 | −0.2295 | 2.1785 | 3.2855 | 0.0555 | 0.5625 | 2.3565 | −0.0615 |
| INPP4B | 685012 \|\| [symbol] | −0.3955 | 0.3025 | −1.3845 | −0.5185 | −1.8855 | −0.3325 | 0.2395 | 0.0035 | 0.2675 | −1.8425 | −0.1365 | 0.4355 | −0.3375 |
| INSM1 | 693435 \|\| [symbol] | 1.807 | 0.307 | −0.108 | 0.481 | 0.938 | 0.972 | 0.904 | 0.201 | −0.082 | 0.413 | 0.487 | 0.722 | 1.121 |
| IQCA | 892963 \|\| [symbol] | 0.397 | 0.907 | −1.009 | 1.385 | 0.01 | −0.297 | 0.609 | 0.96 | −0.795 | 0.601 | 0.003 | 0.202 | 0.679 |
| IQCE | 687430 \|\| [symbol] | −0.442 | −0.28 | −0.483 | −0.262 | 0.555 | −0.035 | 0.605 | −0.987 | −0.131 | 0.217 | 0.37 | 0.243 | 0.218 |
| IQGAP2 | 697172 \|\| [symbol] | −0.5085 | −0.0325 | −0.3675 | 0.2005 | 0.5585 | 0.1745 | 1.1395 | −0.2935 | −0.6905 | 0.5385 | 0.2545 | 0.3415 | 0.2795 |
| IRF4 | 690561 \|\| [symbol] | 3.0015 | 2.5245 | −0.0855 | 2.5045 | −2.2345 | 2.1385 | −0.5515 | 0.8705 | 1.8365 | 2.4685 | −1.3975 | −0.7185 | 2.4685 |
| IRTA2 | 885873 \|\| [symbol] | −0.699 | −0.914 | 1.184 | 0.410 | 0.109 | −1.047 | 0.091 | 0.808 | 1.283 | −0.048 | 0.942 | 1.307 | 1.59 |
| IRX4 | 592616 \|\| [symbol] | 0.1515 | 0.0205 | −0.1105 | 0.8115 | −0.6805 | −0.9475 | −0.4745 | 0.5285 | 1.0685 | −0.7005 | 0.3035 | 1.0735 | −0.4675 |
| ISG20 | 580588 \|\| [symbol] | −0.045 | 0.108 | −1.096 | 0.51 | 0.797 | 0.359 | −0.046 | −1.27 | −1.06 | 0.461 | −1.103 | −0.61 | −1.04 |
| ISGF3 [symbol] | 684521 \|\| [symbol] | −0.599 | −0.214 | −0.969 | 0.052 | 2.62 | −0.001 | −1.119 | −0.175 | −0.275 | −0.849 | −1.973 | −0.858 | −2.208 |
| ITGB4 | 892938 \|\| [symbol] | −0.113 | −0.323 | −0.069 | 0.54 | 0.209 | 0.012 | −0.926 | 0.004 | 0.293 | −0.467 | −0.413 | −0.771 | 1.099 |
| ITPR1 | 680279 \|\| [symbol] | −2.01 | −3.582 | −1.452 | 0.636 | −3.219 | 0.114 | −0.843 | −0.552 | 0.089 | −2.405 | −1.197 | 0.445 | −0.185 |
| JARID1A | 054293 \|\| [symbol] | 0.673 | 0.007 | −1.088 | −1.197 | 0.27 | 0.496 | 0.088 | 0.166 | 0.189 | 0.037 | 0.7 | 0.4 | −0.01 |
| KARCA1 | 092006 \|\| [symbol] | −0.21 | 0.021 | 0.661 | −0.095 | 0.069 | −0.396 | 0.27 | 0.201 | −0.314 | −0.003 | 0.178 | 0.238 | 0.319 |
| KATNA1 | 692605 \|\| [symbol] | 0.2335 | 0.3525 | −0.2785 | −0.2825 | −0.5325 | 0.1935 | 0.8275 | 0.3685 | 0.0755 | −0.0855 | −0.3975 | −0.6535 | −0.1345 |
| KCNC2 | 688570 \|\| [symbol] | 0.043 | −0.232 | 0.607 | −0.063 | −0.086 | 0.138 | −0.074 | 0.032 | −0.148 | 0.356 | −0.5 | −0.002 | 0.508 |
| KCND2 | 683134 \|\| [symbol] | 1.423 | 0.907 | −1.181 | −0.315 | 0.915 | −0.558 | −0.79 | −0.845 | −0.094 | −0.101 | 0.298 | 0.607 | 0.987 |
| KCNK4 | 881761 \|\| [symbol] | 0.111 | 1.142 | 0.475 | 0.415 | 0.785 | −0.151 | 1.806 | 0.525 | 1.189 | 1.107 | 2.16 | 1.119 | −0.493 |
| KCNN4 | 692275 \|\| [symbol] | 1.4125 | −0.4895 | −1.2055 | −0.2815 | 0.8175 | −0.3085 | 0.8495 | −0.9585 | −0.5255 | 0.6305 | 0.4085 | 0.3915 | 0.8905 |
| KCNS3 | 681093 \|\| [symbol] | −0.2175 | −1.1895 | −0.3655 | −1.8155 | −1.3705 | 0.8215 | −0.0855 | 0.2015 | 0.8315 | −0.4465 | −0.1645 | −1.1475 | −0.7545 |
| KCTD15 | 689199 \|\| [symbol] | 2.233 | 1.26 | −0.079 | 1.047 | 0.015 | 0.506 | 1.575 | 0.964 | 1.643 | 0.711 | 0.806 | 0.945 | 0.24 |
| KCTD3 | 886312 \|\| [symbol] | −0.802 | 0.207 | −0.201 | −0.698 | −0.463 | 0.269 | 0.065 | −0.282 | 0.184 | 0.635 | 1.229 | 0.443 | 0.872 |
| KDELR2 | 897248 \|\| [symbol] | −0.91 | −0.707 | 0.406 | −0.137 | −1.003 | 0.315 | 0.233 | −0.31 | 0.153 | 0.889 | 0.663 | 0.237 | 1.124 |
| KIAA0020 | 698703 \|\| [symbol] | 0.424 | 0.194 | −0.045 | 1.038 | 1.169 | 0.25 | 0.757 | 0.306 | −0.388 | 0.459 | 0.228 | 0.611 | 1.262 |
| KIAA0101 | 691153 \|\| [symbol] | 0.6725 | 0.6715 | −0.5975 | 0.6645 | 2.0505 | 1.6975 | 1.2835 | 0.8595 | 1.3405 | −0.1585 | −0.4885 | −0.3935 | 0.8575 |
| KIAA0153 | 694869 \|\| [symbol] | −0.4955 | 0.0215 | 1.0645 | −0.8385 | −0.5535 | −0.0345 | −0.1125 | 0.9155 | −0.8105 | −0.4255 | 1.3225 | 0.5885 | 0.1695 |
| KIAA0182 | 691550 \|\| [symbol] | 0.2735 | 0.6735 | 0.0055 | 0.9585 | 0.0925 | 0.4345 | 1.3075 | −0.0235 | 0.7655 | 0.2085 | 1.1315 | 0.6935 | 0.9225 |
| KIAA0196 | 885973 \|\| [symbol] | 0.5195 | 0.7405 | −0.0275 | 0.3535 | 0.7015 | 0.2895 | 1.3785 | 0.2965 | 0.8745 | 0.7245 | −0.1875 | −0.4215 | 0.3445 |
| KIAA0241 | 692587 \|\| [symbol] | −0.415 | 0.352 | −0.938 | −1.035 | 0.657 | −0.156 | 0.107 | −0.282 | −0.26 | 0.255 | −0.37 | −0.659 | −0.008 |
| KIAA0251 | 882227 \|\| [symbol] | 0.1705 | −0.0425 | 0.3845 | 1.8105 | 0.2425 | 0.7655 | 0.0145 | −0.0495 | 0.3065 | −0.2195 | 0.2875 | 0.3465 | 0.4005 |
| KIAA0274 | 683186 \|\| [symbol] | 0.358 | 0.021 | −0.243 | 0.254 | −3.385 | −0.162 | −0.354 | −0.114 | −1.105 | −1.676 | −1.319 | −0.072 | −1.031 |
| KIAA0515 | 888133 \|\| [symbol] | −0.2465 | 0.0915 | −0.8705 | −1.4525 | 1.4405 | −0.1535 | 0.0185 | −0.2885 | 0.7015 | −0.0635 | 0.3925 | −0.0945 | −0.1355 |
| KIAA0582 | 697645 \|\| [symbol] | 2.2855 | 1.1585 | −0.5235 | 0.3595 | 0.0685 | 0.5005 | 1.2685 | −0.7185 | 0.3285 | −0.0555 | 0.0035 | 0.6755 | 0.5715 |
| KIAA0650 | 884609 \|\| [symbol] | −0.4895 | 0.2435 | −0.0535 | 0.5935 | −1.2135 | −0.0855 | −1.0835 | −0.1425 | −0.1335 | 1.6215 | −0.3295 | −0.2455 | −0.0385 |
| KIAA0703 | 884139 \|\| [symbol] | −0.473 | 0.343 | −2.261 | −0.779 | 0.038 | 0.887 | 0.101 | 1.214 | −0.533 | −1.983 | −0.319 | −0.43 | 0.824 |
| KIAA0830 | 692561 \|\| [symbol] | 1.0795 | −0.3275 | −1.7895 | 1.5125 | 0.8325 | −0.0515 | 0.3935 | −1.0875 | 0.7635 | −0.9595 | −0.5726 | 0.4285 | 0.8005 |
| KIAA0980 | 686300 \|\| [symbol] | 0.9675 | 0.0185 | 0.3045 | 0.3885 | −0.0345 | −0.2915 | 0.2215 | 0.0765 | 0.3825 | 0.9785 | 0.0805 | 0.1895 | 1.5525 |
| KIAA1324 | 890969 \|\| [symbol] | −0.23 | 1.558 | −0.121 | 0.235 | 1.074 | −0.381 | 0.489 | −0.145 | 0.303 | −0.74 | −0.123 | −0.297 | −0.28 |
| KIAA1363 | 696389 \|\| [symbol] | 0.152 | −1.18 | 0.935 | 0.388 | 1.229 | 0.833 | 1.403 | −1.184 | 0.095 | −1.387 | −1.147 | −0.334 | −0.059 |
| KIAA1683 | 864555 \|\| [symbol] | −1.2465 | −0.1845 | −0.4835 | −0.6345 | −0.5795 | −0.2025 | 1.1555 | 0.4245 | 0.5925 | 0.4905 | 0.2905 | −0.0015 | 1.2865 |
| KIF12B | 689788 \|\| [symbol] | 0.0705 | −0.3995 | −0.4865 | 0.2645 | 0.2555 | −0.2985 | −0.0985 | −0.5595 | −0.1485 | 0.3975 | −0.3245 | −0.2825 | 0.1545 |
| KIF24 | 692033 \|\| [symbol] | −0.0476 | −0.5205 | 0.8865 | 0.4405 | −0.0955 | 0.0455 | 0.1445 | 0.0595 | −0.0495 | −0.1245 | −0.3835 | 0.1925 | 0.1605 |
| KIN | 687329 \|\| [symbol] | −0.1375 | 0.2835 | −2.2545 | −2.1685 | −0.9355 | −1.4505 | −1.7115 | 0.1415 | −0.5845 | −0.5205 | 0.0005 | 0.1005 | −0.8055 |
| KIRREL3 | 693623 \|\| [symbol] | 1.4145 | 2.6135 | −0.2295 | −0.2225 | −0.7895 | −0.4895 | −0.3845 | −0.2755 | −0.5335 | −0.6735 | −0.1245 | −0.4355 | 0.3065 |
| KIT | 692519 \|\| [symbol] | 1.4705 | 3.7875 | 1.1255 | 0.1185 | −0.6915 | −1.5775 | 2.0595 | 0.0085 | −1.9145 | −1.7715 | −0.5295 | −0.6925 | −1.2925 |
| KLK5 | 893542 \|\| [symbol] | 1.984 | 1.138 | −0.605 | −0.482 | −0.782 | −0.445 | 1.852 | −0.598 | −0.587 | −0.743 | −0.115 | −0.415 | −0.181 |
| KLK6 | 684796 \|\| [symbol] | 1.407 | 0.358 | 0.171 | −0.107 | −0.314 | −0.932 | 0.702 | −0.589 | −0.811 | −1.117 | 0.488 | −0.439 | −1.015 |
| KLK7 | 895787 \|\| [symbol] | 0.321 | −0.58 | −0.059 | −0.792 | −0.237 | 0.125 | 0.081 | 0.182 | 0.514 | 0.799 | 0.735 | 0.282 | 0.261 |
| KLK8 | 888892 \|\| [symbol] | −0.4155 | 0.1135 | 0.2815 | −0.6155 | −0.4775 | 1.2375 | 0.7005 | 0.3435 | 0.2225 | 0.1455 | 0.5245 | −0.2325 | −0.0876 |
| KNTC2 | 894720 \|\| [symbol] | −0.500 | 2.27 | 1.272 | 0.831 | 0.516 | −0.043 | 3.528 | 0.335 | 0.148 | 2.184 | 1.644 | 1.669 | 1.716 |
| KPNA2 | 668153 \|\| [symbol] | −0.0205 | −0.2545 | −1.1115 | −1.4335 | −0.0055 | −0.5575 | 0.4915 | −0.3605 | −0.7405 | −0.3225 | −0.8065 | −1.2615 | −1.1785 |
| KREMEN2 | 687365 \|\| [symbol] | 2.0475 | −0.4125 | −0.6685 | −0.8665 | −1.2185 | −0.3305 | −0.8526 | −1.3935 | −1.2725 | −1.1425 | −0.8175 | −0.4995 | −1.0445 |
| KRT10 | 594359 \|\| [symbol] | 1.8395 | −0.5015 | −0.2215 | −0.9515 | −1.2535 | −0.4065 | −1.2505 | −1.4785 | −1.9135 | −1.4225 | −1.3805 | −0.9665 | −1.1235 |
| KRT13 | 696433 \|\| [symbol] | 1.257 | 0.819 | −1.153 | −2.243 | −1.558 | −2.427 | −1.577 | −0.298 | −1.345 | −1.056 | −0.372 | −2.097 | −0.178 |
| KRT15 | 694764 \|\| [symbol] | −0.875 | −0.77 | 1.028 | 0.42 | 0.313 | 0.799 | −0.252 | 0.168 | 1.359 | 0.111 | 0.75 | 0.864 | 0.598 |
| KRT17 | 090706 \|\| [symbol] | 1.2475 | 1.3025 | −0.5705 | −1.8585 | −2.4415 | −3.3465 | −1.8185 | −2.4195 | −3.1105 | −3.3015 | −0.4985 | −0.4495 | −2.5715 |
| KRT18 | 889283 \|\| [symbol] | 2.21 | 0.392 | −2.773 | −2.703 | −0.883 | −2.294 | −2.283 | 0.374 | −2.294 | −2.243 | −0.961 | −2.643 | −1.318 |
| KRT23 | 689793 \|\| [symbol] | −0.0305 | −0.1625 | 0.2455 | −0.0805 | −0.2805 | −0.1865 | 0.1005 | −0.1845 | −0.2345 | 0.1475 | −0.0335 | −0.0825 | 0.1715 |
| KRT6B | 696954 \|\| [symbol] | −0.015 | −0.143 | 0.303 | 0.334 | −0.079 | 0.082 | 0.047 | 0.024 | −0.111 | 0.062 | −0.168 | 0.319 | −0.278 |
| KRTAP3-3 | 684073 \|\| [symbol] | 0.2315 | −0.7065 | −0.3845 | −1.5675 | −0.5955 | −1.4175 | −1.8955 | −0.8495 | −0.5805 | −1.5635 | −1.0005 | −2.1295 | −0.4226 |
| KRTHA7 | 883234 \|\| [symbol] | 1.0245 | −0.0485 | −0.8505 | −2.5425 | 0.2345 | 0.0445 | −1.1555 | −0.5455 | 1.8045 | −2.2655 | −0.8055 | −0.6885 | −0.4655 |
| LAMA3 | 689785 \|\| [symbol] | 0.5 | −0.502 | −0.13 | −0.771 | 1.326 | 0.308 | 1.524 | −0.025 | 0.762 | −1.282 | −0.029 | 0.622 | −0.256 |
| LAMB3 | 764891 \|\| [symbol] | 0.088 | −0.208 | 0.064 | −0.407 | 0.093 | 0.054 | 0.747 | −0.254 | −0.399 | 0.577 | −0.281 | 0.216 | 0.099 |
| LASS2 | 695705 \|\| [symbol] | 0.0995 | 0.5735 | −0.6545 | −0.6905 | −1.2105 | 1.1405 | 2.3685 | −0.1505 | −0.2075 | −0.7435 | 0.2805 | 0.0085 | −1.3655 |
| LAX | 682690 \|\| [symbol] | −0.1245 | −1.8795 | −1.2305 | 0.4275 | −1.9145 | −0.9105 | −0.1695 | −1.2775 | 1.1175 | 1.2895 | −0.8015 | −0.1355 | −0.7535 |
| LBR | 681309 \|\| [symbol] | −0.0345 | 1.8895 | −0.6025 | −0.6925 | −1.8975 | 0.3125 | −1.5175 | 1.1585 | −0.2835 | −0.2845 | 1.2995 | −0.9945 | −0.8485 |
| LCE1B | 693798 \|\| [symbol] | 0.1125 | 0.2555 | −0.0015 | −0.4835 | 0.9975 | 0.2895 | −0.1595 | 0.8155 | −0.6415 | −0.3295 | −0.0585 | 0.1035 | −1.3885 |
| LDHB | 663774 \|\| [symbol] | −0.353 | −0.365 | −0.379 | 0.098 | −0.598 | −0.393 | −0.785 | −0.238 | 0.053 | 0.636 | −0.336 | −0.038 | −0.661 |
| LDOC1L | 692299 \|\| [symbol] | 2.893 | 0.203 | −0.988 | 2.649 | 0.007 | 1.902 | 0.767 | 1.797 | 2.78 | −0.143 | −0.569 | 0.51 | 0.255 |
| LETMD1 | 682701 \|\| [symbol] | 0.9575 | 0.3576 | −1.2555 | −1.6695 | −1.8415 | 0.0125 | −0.0125 | −0.9015 | −1.0675 | 0.1525 | 1.2125 | −0.1095 | −1.0325 |
| LFNG | 683275 \|\| [symbol] | 0.306 | −0.235 | −0.638 | −0.529 | 0.11 | −0.022 | 0.418 | −0.079 | −0.048 | 0.084 | −0.888 | −0.616 | −1.796 |
| LGALS2 | 696910 \|\| [symbol] | 0.714 | −0.488 | 0.112 | 0.092 | 1.385 | 1.045 | −0.101 | 0.256 | 0.137 | 0.459 | −0.051 | −0.448 | 0.473 |
| LGTN | 894133 \|\| [symbol] | −0.592 | −1.426 | 0.378 | 0.006 | −0.344 | −0.857 | −0.547 | −0.638 | −0.768 | −1.82 | −1.351 | −1.634 | −0.142 |
| LNX | 688768 \|\| [symbol] | −0.4465 | −0.1225 | −0.9415 | −0.6895 | 0.2435 | −0.2515 | 0.0855 | −0.7025 | −0.2765 | −0.4245 | −0.6995 | −0.2825 | −1.6095 |
| LOC11247 | 687812 \|\| [symbol] | −0.452 | 0.603 | −0.124 | 0.859 | 1.236 | 0.7 | 1.215 | 1.333 | −0.048 | 0.191 | 0.757 | 1.186 | 1.085 |
| LOC11492 | 691804 \|\| [symbol] | −0.366 | −0.329 | −1.188 | 0.593 | 0.611 | 0.417 | 1.005 | −0.422 | −1.403 | −0.657 | −0.239 | −0.369 | −0.02 |
| LOC11497 | 691287 \|\| [symbol] | −0.1285 | −0.1705 | 0.1055 | 0.0505 | −0.4855 | −0.1735 | 0.0355 | −0.3505 | −0.6555 | −0.4035 | 0.0695 | 0.0395 | 0.1865 |
| LOC11621 | 688452 \|\| [symbol] | 2.6975 | −0.3805 | 1.5915 | 2.6325 | 1.5635 | 1.7225 | 3.4515 | 1.3425 | 0.8055 | −1.4805 | 1.2015 | 0.7425 | 2.9965 |
| LOC11644 | 895269 \|\| [symbol] | 1.3565 | −0.9535 | −1.2735 | 0.0865 | −1.2525 | −1.1255 | −1.1415 | −2.0435 | −0.8195 | 0.3335 | 0.2035 | 0.2355 | 0.1025 |
| LOC12022 | 693599 \|\| [symbol] | −0.4095 | −0.1195 | −0.2185 | 0.7555 | 0.4785 | 0.1065 | −0.2135 | 0.2165 | −0.3525 | 0.0775 | −0.1405 | 0.2925 | −0.0735 |
| LOC12422 | 894408 \|\| [symbol] | 0.4945 | 0.8305 | −0.3245 | 0.0285 | −0.6035 | 0.1515 | 0.6965 | 0.1305 | 0.2095 | 0.0005 | −1.0235 | −1.1885 | 0.7245 |
| LOC14654 | 887013 \|\| [symbol] | 0.011 | 0.449 | 0.055 | −0.458 | −0.258 | 0.531 | 0.450 | 1.184 | −0.51 | 0.15 | 0.038 | 0.148 | −0.058 |
| LOC15506 | 683455 \|\| [symbol] | 1.255 | −0.259 | 0.414 | 0.37 | −0.2 | −0.341 | 0.104 | 0.216 | 0.417 | 2.065 | 0.409 | 0.192 | −0.519 |
| LOC20189 | 694158 \|\| [symbol] | −0.1835 | 2.2055 | −0.5895 | 1.8995 | −0.9355 | −0.6885 | 1.1955 | −0.7885 | 3.5895 | −0.9325 | −0.3405 | −1.3055 | −0.6215 |
| LOC25398 | 689291 \|\| [symbol] | 0.687 | 0.313 | 1.085 | 1.549 | 1.139 | 0.323 | −0.084 | 0.657 | 0.625 | −0.103 | −0.385 | 0.32 | 0.348 |
| LOC33990 | 685185 \|\| [symbol] | 0.81 | −0.338 | 0.173 | 0.808 | 1.288 | −0.357 | 0.028 | 0.115 | 0.775 | −0.556 | −0.254 | 0.238 | 0.801 |
| LOC34817 | 686995 \|\| [symbol] | −0.3385 | 1.6555 | 0.0765 | 0.3015 | 0.8275 | −1.5685 | 0.0715 | −0.4595 | 0.2475 | −0.4345 | −0.2805 | −0.6445 | −1.8815 |
| LOC40045 | 683489 \|\| [symbol] | −0.017 | 1.31 | −0.5 | −0.99 | −0.087 | 0.6 | −0.207 | 1.429 | 1.759 | 0.326 | 1.68 | 0.91 | −0.517 |
| LOC44089 | 696189 \|\| [symbol] | −0.492 | 0.526 | −0.055 | −0.446 | −0.627 | 1.05 | 0.539 | −0.087 | −0.813 | 1.481 | 0.884 | 0.487 | 0.742 |
| LOC49386 | 684835 \|\| [symbol] | −0.754 | −0.952 | −1.821 | −0.289 | −1.815 | −0.988 | −1.114 | −0.731 | −0.886 | −1.398 | −0.905 | 0.561 | −1.644 |
| LOC51123 | 897791 \|\| [symbol] | −0.552 | −0.564 | −0.809 | −0.218 | −0.627 | 0.146 | 0.006 | 0.028 | −0.227 | −0.078 | 0.251 | −0.083 | −1.197 |
| LOC51161 | 695670 \|\| [symbol] | 0.9125 | 0.9375 | 0.8385 | 0.3565 | 0.4595 | −0.5945 | −0.9955 | −0.1165 | 0.2825 | −1.1185 | −0.8275 | 0.4985 | 0.0275 |
| LOC51321 | 894489 \|\| [symbol] | −0.7785 | −0.2625 | 1.0255 | −0.4785 | −0.3255 | 1.1255 | −0.0795 | −1.0175 | −0.9295 | −1.2235 | 0.3845 | −0.0165 | −0.3435 |
| LOC51760 | 694904 \|\| [symbol] | −0.3885 | 0.5365 | −1.1135 | 0.2445 | 2.1825 | 1.1935 | 0.3635 | 0.2365 | −0.1155 | 3.9865 | 1.1645 | −0.3985 | −0.3135 |
| LOC81558 | 882974 \|\| [symbol] | −0.398 | −0.376 | −0.888 | 0.396 | −0.729 | −0.731 | −0.626 | −0.141 | −0.123 | −1.569 | 0.232 | 0.016 | −0.684 |
| LOC81569 | 695594 \|\| [symbol] | 0.8165 | 0.5565 | 0.0155 | 0.2115 | −0.3925 | 0.2365 | −0.7875 | −0.5555 | −0.3135 | 1.3425 | 0.5605 | 1.0685 | 0.2175 |
| LOC90355 | 690384 \|\| [symbol] | 0.225 | 0.506 | −0.088 | 0.405 | 0.162 | 0.838 | 1.545 | −0.083 | 0.143 | 0.418 | 0.298 | 0.605 | −0.121 |
| LOC90701 | 691788 \|\| [symbol] | −0.01 | 0.685 | −1.352 | −2.952 | −0.418 | −1.754 | 0.656 | 1.084 | −0.909 | 1.187 | 0.648 | 0.828 | 0.058 |
| LONP | 694635 \|\| [symbol] | −0.3675 | −0.1375 | 0.0755 | −0.1105 | −0.1855 | 0.6335 | −0.6515 | −0.4035 | −0.3445 | 0.0315 | −0.4535 | −0.3195 | 0.7585 |
| LRAP | 994401 \|\| [symbol] | −0.4585 | 9.2255 | 0.4135 | −0.2355 | −0.4825 | 0.2455 | −0.2285 | −0.5525 | −0.2495 | −1.3425 | −0.7105 | −0.6035 | −0.7975 |
| LRBA | 889006 \|\| [symbol] | 0.1775 | −0.4015 | −0.1365 | −0.0885 | −0.3715 | 0.1335 | −0.7845 | −0.3835 | 0.1435 | −1.8025 | −1.3225 | −0.9955 | −0.4275 |
| LRIG1 | 896908 \|\| [symbol] | 1.5155 | 0.7295 | 0.8325 | 0.0895 | 0.3935 | 0.2295 | 0.9405 | −0.4315 | −0.5945 | 1.4255 | 2.1925 | 1.0445 | 0.8785 |
| LRP6 | 888139 \|\| [symbol] | −0.12 | 0.83 | −0.538 | 0.363 | 0.455 | 0.749 | 0.016 | 1.465 | 2.508 | −1.323 | 0.071 | −0.641 | −1.211 |
| LRP8 | 864430 \|\| [symbol] | 0.047 | −0.451 | −0.359 | 0.465 | 0.068 | −0.264 | −0.792 | −0.405 | −0.562 | 0.144 | 0.662 | −0.157 | −0.108 |
| LRRC17 | 689237 \|\| [symbol] | −0.3775 | −0.2005 | −0.1875 | −0.0955 | 0.5885 | −0.0215 | 0.5845 | −0.1945 | 0.4205 | 0.3535 | 0.7915 | 0.5705 | −0.1895 |
| LRRC2 | 695221 \|\| [symbol] | −0.4015 | −0.1315 | −0.2055 | −0.5895 | −0.2545 | 0.3175 | 0.5715 | −0.1755 | −0.0575 | 0.2845 | 0.0855 | 0.1335 | 0.6735 |
| LSM1 | 693186 \|\| [symbol] | 1.3535 | −0.1325 | −2.3325 | −0.6065 | −1.8415 | 0.4695 | −0.0875 | −1.5255 | −1.3125 | 0.7455 | 0.5585 | 0.6215 | −0.0775 |
| LSM8 | 689434 \|\| [symbol] | 0.418 | 2.5 | −0.115 | 0.138 | −0.009 | 0.072 | −0.136 | 0.068 | −0.04 | 2.031 | 0.167 | 0.46 | −0.017 |
| LTB | 895788 \|\| [symbol] | 0.173 | 0.807 | −0.128 | −0.998 | −1.033 | −0.185 | −0.104 | 0.795 | −0.169 | 0.528 | −0.449 | −0.922 | −0.434 |
| LY6D | 688248 \|\| [symbol] | −0.795 | −0.404 | 0.189 | −0.771 | −0.305 | 0.525 | −0.205 | 0.005 | −0.835 | 0.635 | 1.225 | 0.238 | 0.015 |
| LYN | 890653 \|\| [symbol] | −0.212 | −0.485 | −0.018 | 0.063 | 0.053 | −0.18 | −0.645 | −0.166 | 0.124 | 0.239 | −0.267 | −0.028 | −0.288 |
| MA02L1 | 684649 \|\| [symbol] | −0.135 | 2.022 | −0.122 | −0.003 | 0.967 | −0.006 | −0.009 | 0.357 | −0.241 | 2.129 | −0.145 | −0.11 | −0.011 |
| MADP-1 | 687178 \|\| [symbol] | −0.8795 | 3.4025 | 1.0175 | −0.2995 | 0.3045 | −0.4445 | 0.1185 | 0.4265 | 0.1725 | 3.4485 | 0.2125 | −0.5895 | 0.3105 |
| MAGEA12 | 696395 \|\| [symbol] | 0.171 | 2.01 | 0.47 | 0.03 | −0.182 | −0.189 | 0.07 | 0.171 | −0.168 | 2.814 | −0.441 | −0.14 | −0.503 |
| MAGEA2 | 096763 \|\| [symbol] | −0.7075 | 3.0785 | 1.0315 | −0.5915 | −0.7845 | 0.2565 | −0.6435 | 0.2045 | 0.3895 | 3.4295 | 0.8505 | −0.5795 | 0.3675 |
| MAGEA3 | 689285 \|\| [symbol] | −0.2685 | 0.3505 | −0.4595 | −0.9265 | −0.4875 | −0.0825 | −0.3395 | 0.1825 | 0.2405 | −0.9105 | −0.4895 | −0.4855 | 0.2475 |
| MAGEA4 | 890918 \|\| [symbol] | 0.818 | 0.882 | −0.467 | −0.988 | −0.787 | −0.203 | −0.425 | −0.788 | −0.83 | 0.541 | 1.157 | −0.269 | 0.088 |
| MAGED4 | 683069 \|\| [symbol] | −0.0495 | 0.0495 | −0.2295 | −0.1545 | −0.0445 | −0.2105 | −0.2645 | −0.2445 | −8.5165 | 0.2225 | −0.1205 | −0.6995 | 0.2625 |
| MAGI1 | 688247 \|\| [symbol] | 0.5315 | 0.9875 | 2.1345 | −1.7875 | −0.0765 | 0.2455 | 0.5815 | 0.4955 | −1.4895 | 2.0425 | 2.9355 | 2.1215 | 0.9245 |
| MAGI-3 | 892838 \|\| [symbol] | 0.281 | 0.043 | 0.058 | 0.174 | 0.003 | 0.407 | 0.72 | 0.192 | 0.403 | 0.222 | 0.268 | −0.515 | 1.202 |
| MAL2 | 683958 \|\| [symbol] | −0.041 | −1.084 | −1.272 | 0.802 | −1.409 | −1.451 | 1.01 | −0.944 | 1.083 | −0.074 | 1.053 | 0.73 | 0.637 |
| MAN2B1 | 690298 \|\| [symbol] | 2.5085 | −0.2125 | −0.0435 | −0.3535 | 0.0825 | 0.5716 | 0.3855 | −0.6775 | −0.8785 | 0.9455 | −0.1215 | 0.9155 | −0.2805 |
| MAPK8IP2 | 893222 \|\| [symbol] | 0.9145 | 0.5355 | −0.8425 | 3.0705 | −0.4555 | 8.5615 | 3.2375 | 2.2235 | −0.7665 | 0.3385 | 2.0015 | 1.5955 | −0.5235 |
| MARCO | 691594 \|\| [symbol] | 1.113 | −0.227 | 0.982 | 1.615 | 0.026 | −0.46 | 1.547 | 0.969 | 1.938 | −0.489 | 1.174 | 0.398 | 1.476 |
| MARLIN1 | 687688 \|\| [symbol] | −0.763 | −0.114 | −0.463 | −0.384 | 0.188 | 0.435 | 0.206 | −0.524 | 0.562 | 0.37 | 0.341 | 0.154 | 0.055 |
| MASS1 | 687008 \|\| [symbol] | −0.3725 | −0.4365 | 0.2245 | 0.0835 | −0.4305 | 0.6385 | 0.2825 | −0.2225 | 0.0615 | 0.0305 | 0.0885 | 0.1945 | 0.4935 |
| MCM2 | 694711 \|\| [symbol] | 2.4185 | 2.2155 | −0.7305 | −1.7145 | −1.5055 | −0.9265 | −1.3075 | −1.4375 | −1.6885 | −0.8505 | 0.3865 | 1.7315 | 2.9855 |
| MCM3 | 696856 \|\| [symbol] | 0.099 | 0.282 | 1.001 | 0.546 | 1.359 | −0.05 | 0.743 | −1.127 | −1.58 | −0.083 | 0.568 | 1.68 | 0.811 |
| MESP1 | 697846 \|\| [symbol] | −1.1635 | 1.5935 | −1.3085 | −0.5375 | −0.9435 | 0.6015 | 0.3755 | 1.5885 | 1.2185 | −0.4545 | 0.2945 | −1.8055 | −0.3875 |
| METRN | 691541 \|\| [symbol] | −0.2785 | 0.2755 | 0.1315 | −0.3725 | 0.0595 | −0.3065 | −0.3245 | 0.0735 | −0.0985 | −0.4715 | 0.1835 | −0.3305 | 0.1645 |
| MFAP2 | 682204 \|\| [symbol] | 1.785 | 0.211 | 0.475 | 0.151 | 0.366 | 0.952 | 0.916 | −0.298 | 0.336 | −0.815 | −0.217 | 0.118 | −0.13 |
| MFGE8 | 695022 \|\| [symbol] | 1.4515 | 0.1875 | 0.3535 | 0.2375 | 0.0015 | 0.7645 | 0.8795 | −0.1595 | 0.3065 | 8.3865 | 0.3665 | 0.6695 | 0.9005 |
| MGAT4B | 687728 \|\| [symbol] | 0.284 | 0.223 | −0.062 | 0.82 | −0.208 | 1.582 | 0.297 | 0.614 | −0.061 | −0.154 | −0.312 | 0.481 | 0.056 |
| MGC1091 [symbol] | 690398 \|\| [symbol] | 0.3345 | −0.0065 | 0.0785 | 1.4465 | −0.8245 | 2.3105 | 2.4155 | −0.9415 | −0.1505 | 0.2365 | 0.6485 | 1.6275 | 0.8865 |
| MGC1098 [symbol] | 687879 \|\| [symbol] | 0.1375 | −0.7125 | −1.3375 | 0.0445 | 0.0535 | 0.7615 | 0.8315 | 0.0065 | 0.2405 | −0.0085 | 0.1005 | 0.4975 | −0.0135 |
| MGC1124 [symbol] | 880903 \|\| [symbol] | −0.44 | −0.294 | −0.184 | 0.133 | −0.69 | 0.347 | −0.296 | −0.143 | −0.588 | −0.388 | 0.224 | 0.128 | 0.425 |
| MGC1127 [symbol] | 897307 \|\| [symbol] | −0.152 | −0.334 | 0.076 | 0.292 | 0.002 | 0.373 | 0.053 | 0.155 | 0.217 | −0.002 | −0.062 | −0.346 | 0.833 |
| MGC1312 [symbol] | 695012 \|\| [symbol] | −0.249 | −0.56 | 0.663 | 0.992 | −0.538 | 0.165 | 0.468 | −0.116 | 0.623 | 0.048 | 0.33 | 0.51 | 0.427 |
| MGC1318 [symbol] | 887992 \|\| [symbol] | −0.005 | −0.638 | 0.004 | 0.907 | −0.512 | 0.207 | 0.076 | −0.349 | −0.208 | 0.521 | −0.18 | −0.024 | 0.04 |
| MGC1414 [symbol] | 691067 \|\| [symbol] | 0.2015 | −0.0005 | 0.3065 | −0.0085 | 0.6465 | 0.2815 | −0.4745 | 0.2115 | 0.1445 | 0.4415 | 1.6255 | 2.4085 | 0.6685 |
| MGG1428 [symbol] | 695016 \|\| [symbol] | −0.629 | −0.659 | −0.852 | −0.08 | 0.12 | 1.024 | 0.534 | −0.107 | 0.009 | 0.14 | 0.104 | −0.142 | −0.806 |
| MGC1481 [symbol] | 693346 \|\| [symbol] | −0.353 | −0.055 | 0.227 | −0.085 | −0.009 | −0.348 | 0.245 | 0.073 | −0.471 | 0.549 | −0.198 | 0.863 | 0.116 |
| MGC1580 [symbol] | 881291 \|\| [symbol] | −0.1165 | 0.3185 | −0.0095 | −0.7935 | 0.0425 | 0.8895 | −0.2335 | 0.3515 | −0.0395 | −0.0635 | −0.1645 | −0.0245 | 0.0905 |
| MGC1663 [symbol] | 895059 \|\| [symbol] | −0.055 | 0.236 | −0.534 | 0.535 | 0.517 | 0.854 | 0.58 | 0.089 | 0.347 | 0.325 | 0.617 | 0.754 | 0.709 |
| MGC1873 [symbol] | 684966 \|\| [symbol] | −0.285 | 1.028 | −0.278 | −0.183 | −0.067 | 0.045 | 0.209 | 0.966 | 0.969 | 0.203 | 0.796 | 0.134 | 0.522 |
| MGC2053 [symbol] | 692014 \|\| [symbol] | −0.488 | −0.726 | −0.7 | 0.003 | 0.33 | −0.473 | −1.194 | −1.318 | 0.943 | −1.24 | −0.743 | −1.287 | −0.85 |
| MGC2398 [symbol] | 684508 \|\| [symbol] | −0.247 | 0.756 | −0.229 | 0.103 | −0.255 | 0.582 | −0.939 | 0.285 | 1.02 | 1.009 | 0.357 | −0.08 | 0.654 |
| MGC2610 | 685994 \|\| [symbol] | 1.659 | −0.215 | −1.034 | −0.62 | 0.874 | 0.108 | −0.158 | −1.318 | −0.77 | 0.989 | −0.516 | 0.641 | −0.67 |
| MGC2714 | 881884 \|\| [symbol] | −0.279 | 0.288 | −1.072 | −0.484 | 1.34 | 0.382 | −0.094 | −0.59 | −0.707 | 0.921 | −0.144 | 0.55 | −0.423 |
| MGC2716 [symbol] | 882082 \|\| [symbol] | 0.0145 | −0.0305 | −1.2165 | −0.2605 | 1.3405 | 0.2475 | 0.1985 | −0.6975 | −0.9645 | 0.6175 | −0.5556 | 0.7505 | −0.0705 |
| MGC2716 [symbol] | 685085 \|\| [symbol] | −0.2655 | −0.4225 | −0.8765 | −0.7085 | −0.0005 | 0.0525 | 0.4395 | −0.8885 | −0.6975 | 1.5515 | −0.5395 | 0.8035 | −0.5795 |
| MGC2716 [symbol] | 687819 \|\| [symbol] | −0.793 | 0.121 | −0.731 | −0.366 | 1.795 | 0.274 | 0.206 | −0.887 | −0.925 | 1.024 | −0.517 | 0.613 | −0.266 |
| MGC2716 [symbol] | 694146 \|\| [symbol] | −0.6505 | −0.3965 | 0.0065 | −1.3375 | −0.1005 | 0.5745 | 0.2375 | −0.1645 | 0.2665 | −1.1195 | 0.4325 | −0.0845 | −9.6925 |
| MGC2716 [symbol] | 695968 \|\| [symbol] | −0.132 | 0.148 | −0.431 | 0.216 | −0.745 | −0.053 | −0.219 | −1.147 | 0.856 | −0.075 | −0.875 | −0.401 | −0.799 |
| MGC2887 [symbol] | 692023 \|\| [symbol] | −0.504 | 0.197 | −0.791 | −0.385 | −0.417 | 0.081 | 0.307 | −1.037 | −0.367 | −0.342 | 0.265 | −0.312 | −0.857 |
| MGC3212 [symbol] | 693269 \|\| [symbol] | 1.123 | 0.422 | −1.023 | −0.597 | −0.842 | −0.873 | −0.598 | −1.309 | −0.856 | −0.124 | −0.395 | −0.473 | −1.21 |
| MGC3321 [symbol] | 892025 \|\| [symbol] | 0.3445 | −0.5755 | −0.3655 | −0.3385 | −0.3575 | 0.4825 | 0.2595 | 0.4715 | 0.5845 | 1.0865 | 8.6115 | 0.4465 | 0.6375 |
| MGC3484 [symbol] | 692812 \|\| [symbol] | −0.7695 | −0.4155 | −0.3765 | −0.6195 | −0.0135 | −0.7865 | 0.8735 | −0.3205 | −0.4955 | −0.7785 | −0.4695 | −0.5545 | 0.8825 |
| MGC3492 [symbol] | 691558 \|\| [symbol] | −0.944 | −0.453 | −1.288 | −1.962 | −0.638 | −0.403 | −1.609 | −0.897 | −1.292 | −0.198 | −1.881 | −1.299 | −1.824 |
| MGC4021 [symbol] | 895104 \|\| [symbol] | 0.138 | −0.072 | 0.128 | −0.348 | 0.031 | 0.384 | 0.208 | −0.624 | −0.071 | 1.019 | 0.688 | 0.409 | −0.03 |
| MGC4251 | 893489 \|\| [symbol] | −0.4395 | −0.5775 | −0.2965 | 1.0105 | −0.2785 | 0.0635 | −0.0355 | −0.7575 | −0.0925 | −0.0785 | 0.4565 | 0.7345 | 0.6805 |
| MGC4308 | 881305 \|\| [symbol] | 0.597 | 1.661 | −1.196 | 2.389 | −1.595 | −0.05 | 0.005 | 0.489 | −0.825 | −1.13 | −0.325 | 1.261 | −0.037 |
| MGC4800 | 683786 \|\| [symbol] | 0.6405 | 0.0445 | −1.9155 | −0.4855 | −1.8215 | −2.2415 | −1.8625 | −0.3875 | −2.0475 | −1.8345 | −1.2285 | −1.9055 | −1.4885 |
| MGC4659 | 684418 \|\| [symbol] | 0.403 | 1.217 | −0.925 | −0.481 | −0.829 | 0.128 | −0.593 | −0.097 | −0.457 | −0.609 | 0.546 | −0.659 | −1.639 |
| MIA | 695303 \|\| [symbol] | 0.3105 | 1.3785 | −0.6655 | −0.5815 | −0.9745 | 0.0806 | −0.4015 | −0.0865 | −0.9365 | −0.8845 | 0.9465 | −1.0605 | −1.3915 |
| MID1 | 887747 \|\| [symbol] | 0.2395 | 1.4235 | −1.3875 | −0.6885 | −0.9275 | −0.1385 | −1.0855 | −0.8515 | −1.3905 | −1.3215 | 1.0095 | −1.3295 | −1.6515 |
| MID1 | 890115 \|\| [symbol] | −0.413 | −1.28 | 0.494 | 0.045 | 0.038 | 1.259 | 0.708 | 0.088 | −0.339 | 1.042 | 0.147 | −0.131 | 0.594 |
| MID1 | 890599 \|\| [symbol] | 0.5205 | 0.1275 | −0.1845 | 1.2865 | 2.3805 | 1.8095 | 1.3535 | 0.7895 | 1.7315 | −0.3185 | 1.1995 | 0.8205 | 1.6145 |
| MLF1IP | 684863 \|\| [symbol] | −1.5875 | 1.5085 | −0.5135 | −1.0995 | −1.7235 | 0.0735 | 1.8125 | 1.9775 | 2.3345 | 0.6535 | 0.9165 | 0.0675 | 8.4835 |
| MLSTD1 | 689505 \|\| [symbol] | 1.883 | 0.754 | −0.855 | −1.942 | −2.156 | −0.737 | −0.806 | −0.318 | −1.126 | −2.38 | −0.981 | −0.905 | −2.27 |
| MMP1 | 697289 \|\| [symbol] | 0.149 | 1.034 | 0.316 | 0.591 | −0.628 | 0.342 | −0.502 | 0.116 | 0.075 | 0.518 | −0.226 | −0.329 | 0.188 |
| MMP7 | 897274 \|\| [symbol] | −0.5835 | −0.5785 | 0.2065 | 0.0165 | 0.0015 | 0.4685 | −0.5605 | −0.2415 | 0.0065 | 0.7835 | −0.0005 | −0.1375 | 0.3525 |
| MNAT1 | 687030 \|\| [symbol] | −0.6745 | 0.5225 | 0.0385 | −0.2455 | −0.2365 | 0.1205 | −0.5255 | 0.3405 | −0.3305 | 1.3335 | 1.2825 | 1.4575 | 1.2815 |
| MRPL14 | 686347 \|\| [symbol] | −0.777 | −0.313 | 2.533 | 1.222 | 0.267 | 0.926 | −0.268 | 0.099 | −0.011 | −0.085 | 1.072 | 1.652 | 1.577 |
| MRPL15 | 693549 \|\| [symbol] | −0.1505 | −0.1755 | −0.7155 | −0.1075 | −0.1115 | 0.2265 | −0.2715 | −0.2545 | −0.2575 | 1.1595 | 0.1015 | 0.1215 | −0.8525 |
| MRPL27 | 697164 \|\| [symbol] | 0.0725 | −0.0755 | 0.1305 | 0.9835 | −0.2815 | 0.4045 | 0.1425 | 0.2765 | 0.2395 | 0.0825 | −0.3125 | −0.1175 | −0.3516 |
| MRPL34 | 688498 \|\| [symbol] | −0.568 | −0.358 | 0.936 | −0.405 | −0.764 | −0.017 | −0.797 | −0.453 | 4.992 | 1.853 | 1.248 | 0.571 | −0.031 |
| MRPL43 | 883306 \|\| [symbol] | −0.5005 | 0.2185 | −0.0185 | 0.4625 | −0.4735 | −0.2495 | −0.4205 | −0.1295 | 0.0445 | 0.1845 | 0.5905 | 0.8295 | 0.1115 |
| MRPL45 | 687058 \|\| [symbol] | −0.306 | −0.469 | −0.396 | −0.196 | 0.324 | 0.079 | 0.391 | −0.027 | −0.351 | −0.557 | 0.628 | 0.143 | −0.142 |
| MRPL48 | 883204 \|\| [symbol] | −0.2705 | −0.6325 | −1.1235 | 0.1165 | 0.0685 | 0.5115 | 0.4385 | −0.5025 | 0.3495 | −0.1575 | 0.1375 | 0.4405 | −0.2475 |
| MRPL9 | 682558 \|\| [symbol] | 0.3075 | 0.4345 | −0.1505 | 1.0215 | −0.2505 | −0.1825 | 0.6455 | 0.3515 | 0.0005 | 0.2935 | 0.2865 | 0.3236 | 0.5025 |
| MRPS14 | 691200 \|\| [symbol] | 0.244 | −0.186 | −0.059 | 0.903 | 0.231 | 0.817 | 0.953 | −0.573 | 0.485 | 1.045 | 0.507 | 0.774 | 0.813 |
| MRPS15 | 686445 \|\| [symbol] | −0.165 | 0.053 | −0.299 | 0.38 | 0.137 | 0.814 | 0.665 | 0.09 | 0.119 | 0.153 | 0.363 | 0.308 | 0.115 |
| MRPS17 | 695189 \|\| [symbol] | −0.144 | 0.499 | 0.304 | 1.914 | 0.202 | 2.604 | 0.651 | −0.257 | −0.107 | 2.316 | 0.387 | 0.489 | 0.955 |
| MRPS24 | 881503 \|\| [symbol] | −0.1835 | 0.2635 | −0.8505 | −0.3235 | −0.1065 | 0.2785 | −0.4265 | −0.0345 | −0.3715 | 0.5376 | −0.1585 | −0.7145 | −0.1925 |
| MRPS25 | 695169 \|\| [symbol] | 0.2 | 0.475 | −0.476 | 0.03 | 0.244 | 0.378 | 1.753 | 0.865 | 0.075 | 1.156 | −0.439 | −0.423 | 0.287 |
| MRPS31 | 889671 \|\| [symbol] | 0.684 | −0.226 | 0.145 | −0.244 | −0.023 | 0.103 | 0.197 | 0.373 | −0.274 | 0.061 | −0.316 | 0.115 | −0.518 |
| MRPS6 | 692172 \|\| [symbol] | −0.2645 | −0.1495 | −1.0275 | −0.7185 | −0.1075 | 0.8705 | 0.6325 | −0.1895 | 0.3025 | 0.1495 | −0.2895 | −0.6295 | −0.2355 |
| MS4A1 | 890488 \|\| [symbol] | 0.0755 | −0.1195 | −0.4395 | −0.3155 | −0.5045 | 0.0185 | 0.3615 | −0.5625 | 0.3195 | 0.7515 | −0.1005 | −0.0985 | −0.0535 |
| MSH2 | 886029 \|\| [symbol] | 1.704 | −0.992 | −0.057 | 0.629 | 1.371 | 0.638 | 3.082 | 0.206 | −0.368 | −0.18 | −0.278 | −0.567 | −8.183 |
| MSH3 | 592736 \|\| [symbol] | 1.009 | 0.872 | −0.643 | 1.122 | 1.307 | 0.291 | 0.498 | 0.1 | 1.028 | 0.637 | −0.755 | −1.349 | 0.151 |
| MSMB | 695442 \|\| [symbol] | −0.255 | −0.252 | −0.19 | −0.027 | 0.278 | 1.17 | 1.039 | −0.175 | 0.764 | −0.313 | 0.483 | 0.232 | 0.225 |
| MTA1 | 690961 \|\| [symbol] | 0.8185 | 2.2125 | −0.7145 | 1.0115 | 2.1295 | 0.4555 | 0.0115 | −0.8715 | −0.2725 | 0.8895 | 0.0655 | −0.0025 | 0.7935 |
| MTA3 | 684982 \|\| [symbol] | −0.145 | 0.125 | −0.433 | −0.243 | −0.611 | −0.449 | 0.468 | 0.189 | −0.077 | 0.119 | −0.519 | −0.339 | −0.096 |
| MTAC201 | 569442 \|\| [symbol] | −0.064 | −0.045 | −0.132 | −0.546 | 1.469 | −0.52 | −0.501 | −0.014 | −0.44 | 0.649 | 0.028 | −0.303 | 0.277 |
| MTCH1 | 694039 \|\| [symbol] | −0.2295 | 0.6335 | −0.3385 | −0.4585 | −0.6875 | 0.2535 | −1.0475 | 0.7295 | 0.2925 | 0.7165 | 0.0215 | −0.1905 | −0.7095 |
| MTHFD1 | 693532 \|\| [symbol] | 1.8905 | 0.2195 | 0.5855 | 1.5345 | 1.3975 | −1.4555 | 1.6995 | −3.3585 | 0.0935 | −1.3795 | −0.6465 | 0.6655 | 1.2305 |
| MTMR2 | 683517 \|\| [symbol] | 2.0805 | 0.4085 | −0.2195 | 0.9415 | −0.4975 | −0.1445 | 1.6785 | 0.0175 | −0.3325 | −1.3975 | −0.7885 | 0.5895 | −0.5385 |
| MUC1 | 691634 \|\| [symbol] | 0.324 | 0.237 | −1.224 | 0.050 | 0.632 | 0.248 | 0.409 | 0.138 | −0.683 | −0.583 | −1.131 | −0.621 | 0.259 |
| MUC20 | 891484 \|\| [symbol] | 0.334 | 0.064 | −0.413 | 0.182 | 0.251 | −0.26 | −0.361 | −0.232 | −0.155 | 0.093 | 0.249 | −0.357 | −0.582 |
| MUM1 | 884755 \|\| [symbol] | −1.885 | −1.595 | −2.007 | −0.219 | 0.324 | −2.08 | −1.043 | −1.814 | 0.095 | −2.708 | −0.65 | −0.253 | −0.733 |
| MXI1 | 692501 \|\| [symbol] | −0.88 | −0.202 | 0.548 | −0.552 | −2.758 | −1.183 | 0.248 | −0.521 | −0.991 | −0.582 | −0.993 | −0.422 | −1.882 |
| MYB | 693882 \|\| [symbol] | −0.538 | 0.182 | 0.479 | −0.031 | −0.242 | 0.927 | 1.691 | −0.004 | 0.271 | 0.563 | 1.498 | 0.712 | 0.78 |
| MYBL1 | 885207 \|\| [symbol] | 0.674 | 0.165 | 0.726 | 0.586 | 2.076 | 1.472 | 1.09 | 0.75 | 1.082 | 0.955 | 0.175 | −0.723 | 1.023 |
| MYBL2 | 883074 \|\| [symbol] | 0.318 | 0.109 | −1.046 | −0.529 | 0.446 | −0.161 | 0.071 | −0.482 | −0.469 | −1.188 | 0.248 | 0.169 | 0.28 |
| MYO10 | 882216 \|\| [symbol] | −0.4855 | −0.2055 | −0.7005 | −0.0015 | 0.0525 | 0.5515 | −0.5925 | −0.1555 | 0.2135 | −0.4775 | −0.6535 | −0.1875 | −0.4346 |
| MYO6 | 885860 \|\| [symbol] | 0.6975 | 1.5725 | −1.3015 | −1.1875 | −1.9835 | 2.2625 | 0.6065 | 1.0865 | −1.9405 | 0.6965 | 0.8155 | 1.2105 | −0.0325 |
| MYST3 | 772896 \|\| [symbol] | −0.1685 | −0.0825 | 0.2555 | 0.2645 | −0.2515 | −0.2465 | −0.0545 | −0.3395 | −0.1955 | −0.0895 | 0.0375 | 0.2125 | 0.0815 |
| NALP2 | 682203 \|\| [symbol] | 0.4835 | −0.1725 | 0.2335 | −0.2205 | 0.0865 | −0.0315 | −0.0075 | 0.6475 | 1.7715 | 0.5005 | 0.4135 | 0.2475 | 0.8475 |
| NALP6 | 684014 \|\| [symbol] | −0.4005 | 0.4335 | −0.5715 | 0.5955 | 0.8265 | 0.4355 | 0.2185 | 0.3735 | −0.4925 | 0.5045 | −0.0665 | −0.1205 | 0.7295 |
| NANS | 897330 \|\| [symbol] | 0.041 | −0.429 | −0.06 | 0.708 | 0.542 | −0.144 | 1.499 | 0.095 | −0.006 | −0.191 | −0.191 | 0.005 | −0.58 |
| NARF | 887321 \|\| [symbol] | −1.312 | 0.125 | −0.092 | 0.172 | 0.692 | −1.523 | −0.9 | −0.755 | −0.15 | −0.6 | 0.079 | 0.017 | −1.369 |
| NAT2 | 087768 \|\| [symbol] | −0.6375 | −0.8275 | −0.3595 | −0.3035 | −0.0555 | −0.5895 | 0.0355 | 0.2655 | 0.3505 | −0.6405 | −0.1675 | 0.2125 | 0.3815 |
| NAV2 | 885130 \|\| [symbol] | −0.627 | −0.524 | 1.544 | −0.729 | −0.708 | 2.418 | 0.008 | −0.091 | −0.327 | −0.781 | −1.159 | −0.851 | 1.403 |
| NDE1 | 690698 \|\| [symbol] | 0.9225 | 0.1485 | −0.5605 | 0.3565 | −1.7635 | 0.0825 | −0.5515 | −0.8585 | 0.6105 | 0.0215 | −0.6985 | −0.0895 | −1.0375 |
| NDP52 | 584577 \|\| [symbol] | −0.01 | −1.121 | −0.037 | −0.194 | −0.128 | −0.154 | 0.165 | 0.268 | 0.033 | 0.181 | −0.401 | 0.038 | −0.399 |
| NDRG2 | 686705 \|\| [symbol] | 0.458 | 0.337 | −0.196 | 0.926 | −0.907 | −0.155 | 0.128 | −0.521 | 0.365 | 0.918 | 0.216 | −0.029 | 0.371 |
| N0UFA7 | 682915 \|\| [symbol] | 0.1015 | −0.0085 | 0.4615 | 0.5275 | −0.2785 | 0.2475 | 0.4035 | 0.1805 | 0.0825 | 0.5055 | 0.0855 | 0.4665 | 0.2695 |
| NDUFB1 | 695310 \|\| [symbol] | 0.105 | −0.448 | 0.129 | −0.401 | −0.823 | 0.038 | 0.293 | −0.455 | 0.148 | 0.378 | −0.001 | −0.195 | 0.295 |
| NDUFB10 | 681833 \|\| [symbol] | 0.339 | 0.177 | −0.29 | −0.121 | −0.73 | −0.132 | 0.706 | 0.041 | 0.097 | 0.514 | −0.185 | −0.118 | 0.356 |
| NDUFB2 | 688062 \|\| [symbol] | −0.5675 | −0.6805 | 0.0315 | −0.6935 | −0.7185 | −0.3845 | −0.1035 | −0.4615 | 0.0215 | 0.1535 | 0.2925 | 0.0305 | 0.5765 |
| NDUFB5 | 686352 \|\| [symbol] | −0.7405 | 0.1805 | 1.1845 | 0.7345 | −0.6695 | 0.2165 | −0.1685 | 0.6205 | −0.4565 | 0.1175 | 0.7055 | 0.4885 | 0.7855 |
| NDUFB6 | 888628 \|\| [symbol] | −0.2535 | −0.2575 | 0.0175 | 0.8865 | −0.3125 | 0.0285 | 1.5105 | −0.5855 | −0.1865 | 1.7305 | 0.9245 | 0.8475 | 0.6585 |
| NDUFC2 | 698213 \|\| [symbol] | −0.7135 | −0.0675 | −0.2395 | −0.9495 | −0.4835 | 0.1695 | 0.8375 | −0.3365 | 0.0955 | −1.1175 | 0.9685 | 0.0285 | −0.0775 |
| NEBL | 890781 \|\| [symbol] | 1.016 | 0.872 | 0.995 | 9.979 | 0.431 | 0.481 | 0.961 | 0.21 | 0.033 | 1.034 | 0.58 | 0.544 | 0.881 |
| NEK2 | 695375 \|\| [symbol] | −0.964 | −0.991 | 1.218 | −1.42 | −1.715 | −1.025 | −0.406 | −0.896 | −0.711 | −0.672 | −0.705 | −1.957 | −0.803 |
| NEK6 | 595042 \|\| [symbol] | −0.8155 | −1.0125 | −0.5375 | 0.0145 | −0.9765 | −0.8135 | 0.2735 | −0.8105 | 0.9645 | 0.4145 | −0.4815 | −0.1945 | −0.4225 |
| NELL2 | 688574 \|\| [symbol] | −0.0905 | 0.9545 | −1.3055 | −1.4555 | 0.1105 | 0.5055 | 0.3925 | 0.0015 | 0.0955 | 0.3375 | 1.2645 | −0.0655 | −1.0285 |
| NEURL | 881900 \|\| [symbol] | 1.235 | 1.31 | 0.396 | 0.581 | 2 | 0.537 | 1.152 | 0.033 | −0.228 | 0.031 | 0.699 | 0.293 | −0.237 |
| NFE2L3 | 883131 \|\| [symbol] | 0.9415 | 0.6445 | −0.1865 | −1.4125 | −0.2255 | −0.0125 | −0.3385 | −0.3485 | 0.3535 | 1.0485 | −0.1276 | −0.4275 | −0.1905 |
| NFIA | 882198 \|\| [symbol] | 0.04 | −0.616 | −0.044 | 0 | −0.045 | −0.34 | 0.308 | −0.119 | −0.271 | −0.626 | −0.318 | 0.171 | −1.044 |
| NFIB | 681629 \|\| [symbol] | 0.3565 | 0.8845 | 0.2605 | −0.1225 | −0.3265 | 0.2925 | −0.3325 | −0.5225 | 0.1475 | −0.2115 | −0.4725 | −0.9975 | −0.9355 |
| NINJ1 | 684190 \|\| [symbol] | 0.0575 | −0.1545 | 2.3915 | 0.8485 | 0.2015 | 0.1105 | 0.0815 | 0.0785 | −0.0585 | −0.0945 | 0.1565 | 0.4615 | 0.0815 |
| NIPSNAP [symbol] | 697441 \|\| [symbol] | −0.4515 | −0.1965 | −0.4585 | 0.1655 | −0.2595 | −0.2195 | −0.2905 | −0.3485 | 0.0855 | 0.1705 | 2.0885 | 2.0705 | 0.2595 |
| NKX8-2 | 682635 \|\| [symbol] | −0.4505 | −0.0516 | −0.8875 | 0.0775 | −0.3505 | −0.4325 | −0.4505 | −0.1285 | 0.3645 | −0.0935 | 1.9625 | 1.8605 | −0.2125 |
| NME1 | 689970 \|\| [symbol] | −0.046 | −0.421 | 0.229 | 0.646 | −1.158 | −0.287 | −0.298 | −0.428 | 0.014 | −1.008 | −0.39 | −0.014 | 0.021 |
| NME2 | 881655 \|\| [symbol] | −1.3505 | −1.2715 | −0.8415 | −2.3075 | −1.7405 | −1.5795 | −0.5445 | −0.8675 | −0.6025 | −0.4555 | −0.9345 | −0.9595 | −1.2395 |
| NME3 | 682225 \|\| [symbol] | 0.1085 | 0.2905 | 0.1205 | −0.1995 | −0.2585 | −0.3725 | −0.1825 | 0.1575 | 0.3555 | −0.0485 | 0.9975 | 1.1905 | 0.6595 |
| NMES1 | 589520 \|\| [symbol] | 0.296 | 0.215 | −0.471 | −0.706 | 1.208 | 0.573 | 0.005 | −0.003 | −0.65 | 0.079 | −0.019 | −0.346 | −0.481 |
| NMI | 690834 \|\| [symbol] | 0.0315 | −0.3915 | 0.6035 | −0.7005 | 0.2455 | 0.1175 | −0.2275 | −0.0085 | −0.2645 | 0.1005 | 0.4525 | 0.2485 | 0.7635 |
| NOC4 | 691497 \|\| [symbol] | 0.2115 | 0.1515 | 0.0805 | −0.2115 | 0.3245 | 0.1795 | −0.0745 | −0.3545 | −0.4875 | 0.7345 | −0.1085 | 0.2145 | −0.1775 |
| N0LC1 | 889311 \|\| [symbol] | −0.3775 | 0.2755 | 0.2915 | −0.8525 | −1.0325 | 0.2065 | −1.0055 | 0.5675 | −0.5545 | 0.4425 | 0.5825 | −0.1185 | 0.0045 |
| NotFound | 881142 \|\| | 0.0375 | −0.4585 | −0.4325 | −0.1325 | −0.2205 | −0.2185 | −0.3745 | −0.2305 | −0.1635 | 0.1965 | 0.1485 | −0.0135 | −0.0965 |
| NotFound | 881155 \|\| | −0.408 | −0.324 | 0.187 | 0.519 | 0.387 | −0.027 | 0.482 | −0.542 | −0.523 | 0.433 | −0.179 | 0.781 | 0.222 |
| NotFound | 681349 \|\| | 0.655 | −1.414 | 1.525 | 1.431 | 0.064 | 0.357 | 1.51 | −0.372 | 0.618 | 1.282 | −0.166 | −0.338 | −0.852 |
| NotFound | 681357 \|\| | 0.788 | 0.201 | −0.395 | −0.607 | 1.657 | 0.243 | 0.242 | −0.145 | −0.618 | 0.479 | −0.205 | −0.084 | −0.475 |
| NotFound | 681576 \|\| | 0.4065 | 0.5355 | 0.0335 | 0.1295 | −1.1455 | −0.3065 | −0.2645 | 0.6375 | −0.3625 | 1.2155 | 0.0385 | 0.0135 | −0.8725 |
| NotFound | 681608 \|\| | 0.988 | −0.257 | −0.188 | −0.502 | −0.325 | −0.879 | −1.121 | −0.695 | −0.682 | −1.13 | 0.55 | −0.538 | −0.897 |
| NotFound | 881958 \|\| | 0.2555 | −0.6305 | 2.0016 | 2.2595 | 2.3725 | 0.8545 | 1.9565 | 1.2025 | 2.9955 | −1.6195 | −0.8905 | −1.7895 | −0.1705 |
| NotFound | 682219 \|\| | −0.143 | −0.191 | −0.104 | 0.3 | 0.336 | 1.002 | −0.274 | 0.317 | −0.482 | −0.531 | 0.012 | −0.285 | −0.515 |
| NotFound | 682298 \|\| | 1.3895 | 0.5555 | −0.1065 | 0.1955 | 0.1395 | 0.0325 | 0.5335 | −1.0095 | 0.1455 | 0.2695 | 0.2085 | −0.1165 | 0.6495 |
| NotFound | 682699 \|\| | −0.6355 | 0.0365 | −1.9685 | −0.2125 | 0.5405 | 0.6365 | 0.1165 | −1.7805 | −1.1775 | 1.3585 | −0.4375 | 0.6095 | −0.3535 |
| NotFound | 883354 \|\| | −0.0485 | −0.6845 | −0.2805 | 0.1425 | −0.1585 | −0.3185 | 0.1965 | −0.5115 | 0.5125 | −0.3895 | −0.3295 | 0.3355 | 1.0425 |
| NotFound | 883540 \|\| | 0.0535 | −0.0445 | 0.5595 | 0.4415 | −0.0965 | 0.1095 | −0.1285 | 0.2285 | 0.2215 | −0.0115 | −0.1636 | 0.1075 | −0.1055 |
| NotFound | 863552 \|\| | −1.029 | −1.22 | −1.392 | 0.331 | −2.66 | −1.536 | −0.253 | −1.738 | 0.196 | −2.27 | −2.81 | −0.188 | −0.28 |
| NotFound | 683669 \|\| | 0.599 | −0.061 | −0.481 | 0.648 | 0.772 | 0.488 | 0.754 | −0.031 | 0.293 | −0.021 | −0.158 | 0.49 | 0.146 |
| NotFound | 684000 \|\| | −0.518 | −0.761 | 1.088 | 0.45 | 0.295 | 0.928 | −0.28 | 0.211 | 1.498 | 0.004 | 0.703 | 0.881 | 0.459 |
| NotFound | 884333 \|\| | −0.4576 | −0.4425 | 0.1955 | 0.5785 | −0.7035 | −0.0135 | −0.2405 | −0.4555 | 1.1875 | −0.9225 | −0.4285 | 0.2685 | −0.2385 |
| NotFound | 684481 \|\| | 1.051 | −0.875 | 1.339 | 1.777 | −0.799 | −0.601 | 0.476 | 1.381 | −0.496 | −0.097 | −1.139 | −0.984 | −0.159 |
| NotFound | 885169 \|\| | 0.019 | −0.252 | −0.649 | −0.12 | −0.766 | 0.008 | −0.57 | −0.481 | 0.295 | 0.065 | 0.85 | 0.28 | −0.308 |
| NotFound | 885265 \|\| | 0.1675 | 0.7315 | 0.0185 | 0.1165 | 0.2965 | 0.2705 | 0.9965 | −0.4245 | −0.4885 | 0.6645 | 0.0116 | 0.0675 | 0.2415 |
| NotFound | 885572 \|\| | 1.7495 | −1.6445 | −1.2095 | −0.1585 | 2.0565 | −2.2095 | −0.6855 | −1.8835 | −2.0385 | −1.3855 | −1.9295 | −1.5095 | −2.1585 |
| NotFound | 885625 \|\| | −0.151 | −0.478 | −0.705 | 0.889 | 0.078 | −0.063 | −0.934 | 0.023 | 0.035 | −0.621 | −0.426 | 0.223 | −0.501 |
| NotFound | 685756 \|\| | 0.8845 | 1.0525 | −0.3285 | 0.3945 | 0.4465 | 0.8295 | −0.1825 | 0.2805 | 0.2595 | 0.2375 | −0.2075 | 0.0045 | −1.2175 |
| NotFound | 685823 \|\| | −0.926 | −1.383 | −0.07 | 0.294 | 1.711 | −0.627 | 0.705 | −0.053 | 0.933 | −0.946 | −0.371 | 0.948 | −1.144 |
| NotFound | 686027 \|\| | 0.103 | 0.425 | −0.420 | 1.479 | 1.135 | 0.54 | 1.516 | 0.887 | 1.962 | −0.816 | −0.841 | −1.024 | −0.166 |
| NotFound | 686055 \|\| | −0.0985 | 2.0985 | 0.8205 | −1.5295 | 1.4725 | 0.9585 | 0.6005 | 1.0075 | −1.5755 | 1.9245 | 2.5755 | 2.0685 | 1.0945 |
| NotFound | 888477 \|\| | −0.001 | 0.16 | −0.197 | −1.217 | −0.751 | 0.609 | −0.111 | −0.255 | 0.688 | 0.84 | 0.302 | −0.294 | 0.119 |
| NotFound | 686759 \|\| | 0.4595 | 0.2645 | 0.2945 | 1.6135 | 1.5185 | 0.6845 | 1.4555 | 0.7625 | 1.9155 | −0.8395 | −1.0005 | −1.2535 | −0.3925 |
| NotFound | 886948 \|\| | 1.1 | 1.431 | 1.009 | 0.907 | 1.446 | 0.614 | −0.68 | 0.162 | 3.058 | −0.598 | 0.232 | 1.198 | −0.814 |
| NotFound | 687368 \|\| | −0.519 | 0.147 | −0.532 | 0.406 | −1.126 | −0.69 | −1.759 | 0.285 | 0.228 | 0.412 | −0.535 | −0.051 | −0.918 |
| NotFound | 687761 \|\| | −0.3525 | 0.1425 | −0.4205 | −0.4605 | 1.1185 | 0.3405 | 0.0665 | −0.2745 | −0.4235 | 1.3255 | 0.1895 | 0.7525 | 0.0045 |
| NotFound | 888033 \|\| | −0.3795 | −1.1875 | 0.3955 | 0.7505 | −0.2175 | 0.9965 | −0.0765 | 0.1185 | 1.1715 | −1.8005 | −0.9785 | −1.5676 | −1.3855 |
| NotFound | 888163 \|\| | −0.4345 | 1.2685 | 1.2815 | 2.1845 | 2.2165 | 1.6955 | 0.9325 | 1.6545 | 2.2015 | −1.0685 | −0.9925 | −1.0875 | −1.1195 |
| NotFound | 888264 \|\| | 0.2165 | 0.5045 | 0.3805 | 1.8605 | 2.1205 | 0.5805 | 1.0215 | 0.4315 | 2.2435 | −0.7645 | −0.8665 | −1.0335 | −0.7105 |
| NotFound | 888843 \|\| | −1.0795 | −1.1715 | −0.2125 | 1.7535 | 0.8325 | −0.3665 | 0.7425 | −0.7925 | 1.2525 | 0.5975 | −0.4055 | 0.0185 | −0.5445 |
| NotFound | 688954 \|\| | −0.047 | −0.262 | 0.078 | 1.812 | 1.422 | 0.599 | 1.285 | 0.324 | 1.443 | −0.269 | −0.98 | −0.61 | −0.652 |
| NotFound | 688988 \|\| | −0.2855 | 0.2225 | 0.0015 | 0.5235 | 1.5675 | −0.2585 | 0.6835 | 0.3975 | 1.4816 | −2.1765 | −2.5585 | −2.6305 | −1.9025 |
| NotFound | 689368 \|\| | −0.5995 | 0.2425 | 1.0615 | −0.6825 | 0.2875 | 0.2235 | 0.1445 | −0.0095 | 0.0585 | 0.0345 | 0.8885 | 0.9895 | 0.9055 |
| NotFound | 689732 \|\| | 0.5015 | −0.6005 | 1.2935 | 2.6375 | 2.2455 | 1.3855 | 2.4395 | 0.8555 | 2.5405 | −2.2445 | −1.7705 | −1.4065 | −0.7385 |
| NotFound | 690073 \|\| | 0.6195 | 0.5895 | 0.9835 | −0.8095 | 1.1885 | 0.2176 | 0.0335 | 0.2825 | 0.3695 | 0.0205 | −0.6565 | −1.7245 | −0.8825 |
| NotFound | 800533 \|\| | −0.746 | −0.106 | −0.523 | −1.55 | −0.998 | −0.485 | −0.693 | −0.031 | −0.111 | −0.23 | −0.588 | −1.026 | −1.185 |
| NotFound | 69057 [symbol] \|\| | 0.023 | −0.055 | 0.146 | 0.129 | 0.032 | −0.25 | −0.005 | 0.141 | −0.442 | 0.167 | 0.089 | 0.053 | −0.039 |
| NotFound | 890683 \|\| | −0.111 | −0.323 | −0.37 | −0.244 | −0.522 | 0.251 | 0.897 | −0.774 | −0.369 | −0.247 | −0.14 | 0.33 | 0.041 |
| NotFound | 691307 \|\| | 0.4835 | 0.4105 | 0.0595 | 1.9316 | 1.4245 | 0.7345 | 1.4545 | 0.5855 | 1.7655 | −0.3275 | −0.6245 | −0.8045 | −0.1175 |
| NotFound | 691444 \|\| | −0.4585 | −0.4855 | −0.4435 | −0.5595 | −0.6395 | −0.4035 | 0.1155 | 0.4545 | −0.2925 | 0.5615 | −0.3425 | −0.2405 | −0.9275 |
| NotFound | 691405 \|\| | −0.007 | −0.187 | 0.524 | 0.487 | 0.485 | 0.063 | −0.013 | −0.012 | −0.209 | −0.17 | 0.044 | 0.618 | 0.193 |
| NotFound | 691652 \|\| | −0.7255 | −0.0145 | 0.3315 | −0.4445 | 0.2245 | 0.1455 | 0.4415 | 0.4466 | −0.1365 | 0.0505 | −0.0555 | −0.3515 | 1.3605 |
| NotFound | 681783 \|\| | 0.1155 | 0.5225 | 0.1745 | 0.2195 | −0.0885 | 0.4816 | 0.6135 | −0.4285 | 0.6785 | −1.0585 | −0.3595 | −0.3705 | −0.8205 |
| NotFound | 692077 \|\| | −0.0025 | 0.1425 | −1.2305 | −0.9705 | 0.7215 | 0.2805 | −0.6815 | 0.0415 | −0.3485 | −0.8205 | −0.7545 | −1.1405 | −0.4025 |
| NotFound | 692188 \|\| | 0.1476 | 0.2495 | 0.1315 | 1.1716 | 0.5395 | 0.0205 | 0.5375 | −0.0905 | 0.2395 | 0.5105 | 0.5945 | 0.8415 | 0.3025 |
| NotFound | 892466 \|\| | 0.154 | −0.026 | −0.067 | −0.037 | 1.009 | 0.543 | −0.191 | −0.923 | −0.104 | 0.684 | −0.434 | −0.785 | −0.05 |
| NotFound | 692727 \|\| | 0.567 | 0.096 | −0.046 | 0.723 | −0.511 | −0.897 | 1.819 | 0.163 | −0.051 | 0.699 | 0.623 | 0.351 | −0.092 |
| NotFound | 692860 \|\| | 0.3445 | −0.9745 | 1.6195 | 1.6405 | 0.5985 | 0.6795 | 1.2225 | −0.1435 | 0.7875 | 1.2075 | −0.2915 | −0.0095 | −0.6105 |
| NotFound | 692931 \|\| | 0.0675 | −0.4785 | −0.6035 | 0.9825 | −0.8295 | 0.1985 | −0.8395 | 0.0225 | −0.4785 | −0.2615 | 0.8465 | 1.1135 | 0.2385 |
| NotFound | 693121 \|\| | 0.5545 | −0.6505 | −0.4835 | 1.4525 | −0.0925 | −0.0875 | 1.0005 | −0.4705 | 0.8965 | −2.3485 | −1.1575 | −0.3575 | 0.3455 |
| NotFound | 693387 \|\| | −0.4615 | 0.8375 | −0.7055 | −0.7515 | −0.2065 | −0.6775 | 1.1705 | −0.5635 | −0.5275 | 0.3595 | −0.5085 | −1.0205 | 1.5755 |
| NotFound | 693511 \|\| | −0.782 | −0.127 | −2.53 | −1.278 | −1.036 | −2.489 | −2.119 | −1.273 | −1.353 | 2.198 | 0.01 | 1.205 | −2.841 |
| NotFound | 693741 \|\| | 1.905 | 0.244 | −0.554 | −0.805 | −1.287 | −0.736 | −0.034 | −0.847 | 0.395 | −0.839 | −0.389 | −0.577 | −0.572 |
| NotFound | 894011 \|\| | −0.3015 | 0.0215 | 0.4655 | 0.0195 | −0.0195 | −0.1105 | 0.0215 | 0.0605 | −0.1345 | 0.0805 | −0.0136 | 0.0015 | −0.1985 |
| NotFound | 894287 \|\| | 1.034 | 1.527 | −0.465 | −0.246 | 1.055 | 2.073 | 0.401 | 1.668 | 0.392 | 1.928 | 0.416 | −0.014 | −0.281 |
| NotFound | 694458 \|\| | −0.962 | 0.436 | −0.14 | −0.264 | 0.013 | 0.211 | −0.58 | −0.009 | −0.696 | −0.795 | −0.461 | −0.486 | −0.678 |
| NotFound | 695199 \|\| | 0.3795 | 0.0995 | 2.3805 | 0.1095 | −0.5565 | 0.9245 | −0.6455 | 1.8125 | 1.7535 | −0.0905 | −0.4925 | −0.2185 | −0.7025 |
| NotFound | 895280 \|\| | 0.173 | 0.148 | −0.495 | 0.289 | −0.243 | 0.207 | −0.333 | −0.089 | −0.008 | 0.43 | −0.2 | 0.19 | −0.078 |
| NotFound | 895667 \|\| | −0.646 | 1.472 | 1.42 | 2.551 | 2.576 | 2.046 | 1.295 | 2.232 | 2.617 | −1.715 | −1.604 | −1.475 | −1.496 |
| NotFound | 896120 \|\| | 1.7505 | −0.5195 | −1.1485 | 1.5185 | −0.7335 | 0.9795 | 0.4805 | −0.1405 | 2.1995 | −0.7195 | 0.4005 | 1.2235 | 0.4845 |
| NotFound | 696388 \|\| | 0.343 | −0.38 | 0.196 | −0.257 | −1.044 | −0.801 | −0.148 | −0.512 | −0.664 | −0.691 | −0.314 | −0.115 | −0.443 |
| NotFound | 695512 \|\| | 0.2905 | −0.1645 | 1.0595 | 0.5895 | 0.7725 | 0.4035 | 0.4325 | 0.5645 | −0.1465 | 0.7425 | 0.6705 | 1.0205 | 0.6905 |
| NotFound | 695782 \|\| | −0.0675 | 0.0255 | 0.6595 | 0.3385 | 0.1215 | −0.0885 | −0.2715 | 0.4815 | −0.1365 | −0.0185 | 0.1135 | 0.3225 | 0.3345 |
| NotFound | 697127 \|\| | 0.135 | 0.223 | 0.19 | 1.64 | 1.555 | 0.461 | 1.498 | 0.742 | 1.91 | −0.614 | −0.939 | −1.254 | −0.227 |
| NotFound | 897197 \|\| | −0.2235 | −0.3175 | 0.0025 | −0.2905 | 0.3185 | 0.4785 | 0.3835 | 0.0465 | 0.2035 | −0.8045 | 0.6955 | 0.2755 | 0.0335 |
| NotFound | 897297 \|\| | 0.696 | −0.069 | 0.963 | 0.72 | −0.392 | −0.35 | 0.561 | −0.353 | 1.31 | −0.678 | −0.186 | −0.387 | 0.817 |
| NotFound | 697403 \|\| | −0.534 | −0.117 | −0.019 | −0.247 | −0.642 | −0.495 | 0.767 | −0.466 | −0.678 | −0.670 | −0.464 | 0.006 | −0.699 |
| NPDC1 | 682819 \|\| [symbol] | −0.4265 | 0.0615 | 0.4785 | 0.6255 | 0.3085 | −0.1215 | 0.1325 | −0.1555 | −0.0055 | −0.2175 | 0.2845 | 0.3985 | 0.1835 |
| NPEPPS | 687914 \|\| [symbol] | 0.07 | −0.699 | −0.298 | 0.08 | −0.081 | 0.482 | 0.852 | −0.686 | 0.421 | −0.934 | −0.581 | 0.205 | −0.28 |
| NPY | 683068 \|\| [symbol] | −0.689 | −0.286 | −0.075 | −0.106 | −0.997 | 0.297 | −0.979 | 0.088 | 0.557 | −0.892 | 0.269 | −0.056 | −0.478 |
| NQO3A2 | 897754 \|\| [symbol] | −0.199 | 0.091 | −1.186 | −0.313 | 0.363 | −0.087 | 0.286 | 0.351 | −0.222 | −0.957 | −2.018 | −1.958 | −0.822 |
| NRAS | 695502 \|\| [symbol] | 1.071 | 0.857 | 1.31 | −2.162 | −0.395 | −2.05 | −2.07 | 0.441 | 0.73 | −0.313 | 0.073 | −0.227 | 0.06 |
| NRIP1 | 684742 \|\| [symbol] | −0.1305 | −0.5985 | −1.3865 | 1.0815 | −0.2595 | −0.1695 | −0.1595 | −1.3335 | 1.0895 | 0.8465 | −0.4255 | 0.3435 | 0.0075 |
| NTN4 | 686487 \|\| [symbol] | −0.5385 | 0.0365 | 0.0865 | −0.2695 | 0.1905 | −0.6275 | 0.2345 | −0.1275 | −0.1165 | 0.6785 | 0.2375 | 0.1875 | 0.8605 |
| NUCB2 | 681359 \|\| [symbol] | −0.7585 | −0.9395 | −0.5815 | −1.3575 | −1.1575 | −0.6095 | −1.0265 | −0.5485 | −0.5545 | 0.3245 | −0.7305 | −0.4235 | −1.7285 |
| NUDT5 | 695608 \|\| [symbol] | −0.607 | 0.143 | 0.137 | −0.743 | 0.7 | 0.056 | −0.744 | 0.688 | −0.338 | 0.11 | 0.138 | 0.185 | −0.299 |
| NUDT6 | 686517 \|\| [symbol] | −0.347 | 1.607 | −0.244 | −0.048 | −0.324 | 0.388 | −0.289 | −0.018 | −0.002 | −0.279 | 0.008 | 0.062 | −0.204 |
| OCIAD2 | 695560 \|\| [symbol] | −0.4645 | 0.0345 | 0.0765 | −0.6945 | −0.8095 | −0.1695 | −0.1395 | −0.3155 | −0.7145 | 0.1565 | 0.8225 | 1.2665 | 0.2825 |
| OFD1 | 683518 \|\| [symbol] | −0.4355 | −1.1555 | −3.3235 | 0.8935 | −2.3235 | −1.8035 | −1.1595 | 0.8985 | −0.1315 | 0.9085 | 1.7145 | 2.4325 | −1.8155 |
| OGFRL1 | 689994 \|\| [symbol] | 1.272 | 2.174 | 1.933 | 1.379 | 0.007 | 2.298 | −0.288 | 2.539 | −0.029 | −0.781 | 0.379 | 1.174 | 1.273 |
| OPLAH | 694256 \|\| [symbol] | 1.8235 | 1.0125 | 0.1945 | −0.0825 | −0.7585 | −0.3205 | −0.1155 | 0.5625 | 0.0545 | 7.3905 | 0.9175 | −0.5555 | 0.6175 |
| ORMDL3 | 683021 \|\| [symbol] | 0.041 | −0.489 | −0.088 | 0.57 | −1.375 | 0.643 | −0.286 | −0.428 | 0.506 | −0.911 | −0.106 | −0.634 | −1.169 |
| OSBPL3 | 891048 \|\| [symbol] | 0.002 | 0.158 | 0.215 | −0.247 | 0.315 | −0.544 | 0.131 | 0.148 | −0.117 | 0.73 | 0.531 | 0.164 | 0.109 |
| OSR2 | 881734 \|\| [symbol] | 0.1455 | −0.2716 | −0.6635 | 0.6385 | −0.2815 | 0.8765 | 0.7565 | −0.2965 | −0.2165 | 0.9415 | −0.3395 | 0.4465 | 0.0275 |
| OSTF1 | 664940 \|\| [symbol] | −0.448 | 0.477 | 1.801 | 1.814 | −0.567 | 0.318 | 0.603 | −0.711 | 0.392 | 0.758 | 1.424 | 1.324 | 1.995 |
| P2RX1 | 661440 \|\| [symbol] | −0.2955 | −0.4465 | −0.5315 | 2.2485 | 0.6075 | −0.5835 | 0.4175 | −0.7125 | 0.1005 | 1.0055 | −0.5005 | 0.6415 | 0.3575 |
| P2RY2 | 696219 \|\| [symbol] | 1.562 | 1.4 | −0.129 | 0.992 | 1.649 | 0.165 | −0.071 | −0.533 | −0.12 | −0.381 | 0.106 | 0.691 | 0.484 |
| PACAP | 697667 \|\| [symbol] | 0.162 | 0.237 | 0.122 | −0.289 | 0.043 | −0.149 | −0.258 | −0.038 | −0.377 | −0.108 | −0.097 | 0.019 | 0.702 |
| PADI2 | 888464 \|\| [symbol] | 2.8815 | 1.4605 | −0.7235 | 1.6095 | 1.4155 | 1.1825 | 1.4975 | 1.0765 | 0.9235 | 3.1595 | 2.4215 | 2.1705 | 0.8995 |
| PAPD5 | 889131 \|\| [symbol] | 0.116 | −0.529 | 1.106 | 0.435 | 0.282 | 0.505 | −0.135 | −0.038 | −0.004 | −0.379 | −0.023 | 0.512 | 0.775 |
| PAPSS2 | 888187 \|\| [symbol] | −0.058 | −0.57 | −0.495 | 0.322 | 0.021 | 1.123 | 0.897 | −0.266 | 0.352 | −0.49 | 0.205 | −0.248 | 0.001 |
| PARD8A | 692812 \|\| [symbol] | −0.101 | 0.864 | 0.851 | −0.236 | 0.001 | −0.341 | −0.505 | 0.502 | 1.217 | 0.107 | 0.367 | 1.234 | −0.606 |
| PARP1 | 683432 \|\| [symbol] | 0.155 | −0.396 | −0.995 | −1.075 | −0.381 | 0.34 | 0.49 | −0.441 | 0.127 | −0.041 | −0.514 | −0.785 | −0.108 |
| PARP9 | 895188 \|\| [symbol] | −0.5125 | 1.0395 | −0.0845 | 0.7195 | −0.8785 | −0.1085 | 1.0775 | 0.3915 | −0.0485 | −0.4545 | −1.0195 | −0.6645 | 0.4565 |
| PAXIP1L | 684118 \|\| [symbol] | 0.5815 | 0.8055 | −0.2365 | 1.0625 | 0.5515 | 0.2685 | 1.1115 | −0.0925 | −0.2465 | 0.0345 | −0.5815 | −0.6025 | 0.2345 |
| PBX3 | 692245 \|\| [symbol] | 0.0605 | 0.3345 | −0.2455 | 1.2485 | 1.5745 | −0.4165 | 0.4835 | 0.6555 | −0.0925 | −0.5085 | −0.7855 | −0.3645 | −0.0105 |
| PCDHB11 | 681640 \|\| [symbol] | 1.928 | 2.38 | −1.17 | −0.024 | −0.534 | 1.823 | 3.342 | 0.697 | −0.928 | 1.106 | −1.078 | −1.453 | 1.542 |
| PCDHB16 | 697521 \|\| [symbol] | −0.468 | −0.807 | −1.383 | −0.551 | 0.054 | −0.201 | 0.088 | −0.835 | −0.027 | −0.883 | −0.791 | −1.382 | −0.508 |
| PCDHB2 | 884692 \|\| [symbol] | −0.3005 | −0.2715 | −0.1435 | −0.6915 | −0.3515 | 0.3245 | 0.4485 | 0.3915 | 0.0495 | 0.6385 | −0.0195 | −0.0355 | 0.2005 |
| PCM1 | 685108 \|\| [symbol] | −0.1735 | −0.0885 | 0.4725 | 0.5305 | −0.1985 | 0.1015 | 0.2705 | −0.0455 | −0.1685 | 0.5415 | 0.1445 | 0.7335 | −0.0825 |
| PCNA | 883820 \|\| [symbol] | −0.252 | 0.368 | 0.189 | −1.111 | −0.188 | −0.201 | −0.111 | −0.52 | −0.190 | 0.577 | 0.161 | −0.311 | 0.408 |
| PDC01 | 687556 \|\| [symbol] | −0.0195 | −1.2955 | −1.9935 | −0.0305 | −0.0735 | −0.7095 | −0.7205 | −0.1015 | 1.2705 | −0.8225 | −0.8505 | −1.2335 | −0.2285 |
| PDC010 | 894505 \|\| [symbol] | −0.01 | −0.141 | 3.7 | 0.156 | −0.49 | 0.443 | 0.194 | 0.476 | 0.142 | 0.817 | 0.13 | 0.038 | 0.353 |
| PDCD4 | 892083 \|\| [symbol] | 0.4025 | 0.1455 | 0.0275 | 0.7215 | 0.3625 | 0.8135 | 0.5585 | 0.4845 | −0.0715 | 1.2275 | 0.3165 | 0.2015 | 0.0045 |
| PDCD5 | 882521 \|\| [symbol] | −0.3725 | −1.0585 | −0.4455 | 0.9265 | −0.5455 | −0.7685 | −0.5295 | 0.5655 | 0.7435 | −0.6615 | 0.2635 | 0.6875 | −1.2425 |
| PDCD8 | 687693 \|\| [symbol] | 0.0075 | −0.1755 | −0.0135 | 0.4115 | −0.4435 | 0.8815 | 0.3276 | 0.1055 | −0.0465 | 0.3145 | 0.3825 | 0.2405 | 0.8815 |
| PDE4DIP | 689720 \|\| [symbol] | 0.7315 | −1.5565 | 0.1275 | 1.2675 | 0.3245 | 1.0055 | 0.8745 | 0.4525 | 0.2935 | −0.3825 | −0.2535 | 0.1725 | 0.8585 |
| PDHA1 | 684251 \|\| [symbol] | −1.518 | −0.022 | −1.096 | 1.701 | 1.006 | 0.519 | 0.058 | 3.173 | −1.605 | −2.293 | 1.37 | 0.28 | −2.104 |
| PECI | 889878 \|\| [symbol] | 0.0915 | −0.0975 | 0.0785 | −0.3275 | −0.6825 | −0.6925 | −0.6365 | −0.3115 | −0.5115 | 0.1625 | −1.0755 | −0.7905 | −0.7305 |
| PEG10 | 887554 \|\| [symbol] | 1.2945 | 1.2655 | −1.2295 | −0.4265 | 1.1865 | 0.2795 | 1.0245 | −0.7245 | −0.0825 | 0.5595 | 0.2915 | 0.3845 | 0.1565 |
| PER2 | 888213 \|\| [symbol] | 0.527 | −0.82 | −0.408 | −1.085 | −0.45 | 0.594 | 1.178 | −0.321 | 0.005 | −0.375 | −0.155 | −0.416 | 0.24 |
| PERP | 683121 \|\| [symbol] | −0.7555 | 0.4425 | −1.0415 | −1.4725 | −0.6255 | −0.4715 | −0.1415 | −0.4205 | −0.1775 | −1.0165 | −0.6485 | −0.8915 | −0.0025 |
| PEX1 | 686946 \|\| [symbol] | −0.029 | 0.24 | 0.322 | −0.05 | 0.609 | 0.214 | 0.088 | 0.14 | 0.612 | −0.218 | 0.433 | 0.581 | −0.115 |
| PFAAP5 | 683100 \|\| [symbol] | 0.0055 | −0.1965 | −0.5285 | −0.0055 | −0.1665 | −1.0345 | −0.2165 | −0.9125 | 0.4815 | 0.8505 | −0.8655 | −0.7175 | −1.0365 |
| PFDN2 | 687458 \|\| [symbol] | −0.302 | −0.137 | 0.402 | −0.899 | −0.277 | −0.372 | −0.452 | 0.724 | −0.822 | −0.435 | −0.095 | −0.151 | −0.874 |
| PGM1 | 681278 \|\| [symbol] | −0.5265 | 0.2805 | −0.1225 | −0.1035 | −0.5835 | 0.1455 | −0.1875 | 0.3335 | 0.6775 | −0.2745 | 0.1085 | 0.2595 | 0.3075 |
| PGR1 | 889407 \|\| [symbol] | −0.6715 | 0.0165 | −0.0095 | 0.6115 | 0.3135 | 0.5505 | 0.2075 | 0.1805 | 0.8015 | 0.2245 | 2.0145 | 2.2325 | 0.4005 |
| PHAX | 682593 \|\| [symbol] | 0.021 | 0.559 | −1.288 | −1.06 | 0.301 | 0.794 | 1.11 | −0.445 | −0.685 | 0.148 | 0.243 | 0.897 | −0.729 |
| PHGDH | 582625 \|\| [symbol] | −0.5355 | −0.6025 | 1.0495 | −0.6335 | −1.9675 | 0.4355 | −1.8215 | 1.2785 | 1.8755 | −1.0665 | 0.4815 | 0.4375 | 1.4035 |
| PHYH | 689499 \|\| [symbol] | 0.1245 | −0.0305 | −0.4935 | −0.8805 | −0.6005 | −0.2355 | 0.6705 | −0.2595 | 0.4025 | −0.4316 | −0.4005 | 0.0695 | −0.1916 |
| PI85PA | 891558 \|\| [symbol] | 0.1295 | −0.2165 | 0.0835 | −0.4485 | 0.2025 | −0.5205 | 1.1335 | −0.5265 | −0.7755 | −0.3445 | −0.3435 | 0.8595 | 0.3805 |
| PIGT | 688448 \|\| [symbol] | 0.342 | −0.331 | −0.272 | −0.112 | 1.487 | −0.269 | −0.837 | 0.054 | 0.107 | 0.22 | −0.423 | 0.281 | 1.403 |
| PIM2 | 697074 \|\| [symbol] | 0.0965 | 0.6395 | −0.5045 | 0.5945 | 0.8295 | 0.0855 | 1.4195 | 0.1695 | −0.0435 | 0.4705 | 0.8055 | 1.0355 | 0.2925 |
| PISD | 696650 \|\| [symbol] | 0.583 | 1.615 | −1.745 | 0.118 | 0.645 | −1.191 | 1.898 | −0.735 | −0.184 | 1.218 | 0.085 | 1.537 | 2.071 |
| PITPNC1 | 894234 \|\| [symbol] | −0.3855 | 1.6015 | 0.4405 | −0.0785 | −1.0535 | 0.3815 | 1.0065 | 0.0025 | 0.1645 | 1.3905 | 0.5835 | 1.1885 | 1.0385 |
| PKIB | 681591 \|\| [symbol] | 0.542 | −0.121 | −0.078 | 0.984 | 0.095 | 0.367 | −0.356 | −0.701 | −0.158 | 0.235 | −0.426 | 0.009 | 0.577 |
| PKMYT1 | 688037 \|\| [symbol] | −0.088 | −0.174 | −0.021 | 0.388 | −0.817 | −0.15 | −0.324 | 0.717 | 0.183 | −0.231 | −0.018 | 0.513 | −0.828 |
| PLA2G4B | 895710 \|\| [symbol] | 1.2575 | −0.1605 | −0.2685 | 0.9905 | 1.0385 | 0.0195 | 1.6615 | −0.1465 | 0.6915 | −0.1245 | −0.3065 | −0.9125 | 1.0925 |
| PLAC8 | 892243 \|\| [symbol] | −0.034 | −0.13 | −0.07 | −0.03 | 0.164 | 0.282 | 0.315 | −0.167 | −0.055 | 0.436 | −0.261 | 0.27 | 0.047 |
| PLCB1 | 681242 \|\| [symbol] | −0.2405 | −0.0145 | −0.9515 | 0.8795 | 1.9745 | 0.3555 | 0.3855 | 0.2295 | −0.3675 | 0.8755 | 0.2495 | −0.1175 | 0.8685 |
| PLCXD1 | 686574 \|\| [symbol] | −0.214 | 1.717 | 0.123 | 0.993 | 0.335 | 0.044 | 0.977 | 0.038 | 0.001 | 0.342 | 0.531 | −0.032 | 0.824 |
| PLEK2 | 686904 \|\| [symbol] | 0.3205 | 0.2695 | 0.1875 | −0.6655 | −0.1595 | 0.6755 | 1.1175 | −0.2045 | 0.6705 | 0.5415 | 0.1755 | −0.4675 | 0.0595 |
| PLO0 | 890311 \|\| [symbol] | 1.3265 | 0.3835 | 1.2905 | −0.1285 | −0.1075 | −0.3215 | 0.0345 | 0.1075 | −0.1865 | 0.3255 | 0.0855 | 0.3615 | −0.5245 |
| PNAS-4 | 895278 \|\| [symbol] | 2.2795 | 0.8475 | −1.1085 | 1.7165 | −1.4875 | 0.1795 | 1.7945 | −0.1975 | −0.1835 | −0.3115 | −0.7845 | 0.0385 | −1.3255 |
| PNLIPRP2 | 882823 \|\| [symbol] | −0.7225 | −0.0575 | −0.4155 | 0.1785 | 0.2395 | 0.7785 | 0.1815 | −0.5105 | 0.3385 | −0.6475 | 1.2125 | 0.9835 | −0.5265 |
| PNMA3 | 695191 \|\| [symbol] | −0.1565 | −0.0895 | 0.0745 | −0.2365 | 0.8525 | −0.2875 | −0.0905 | −0.0015 | 0.2855 | 0.1735 | 0.1516 | 0.2985 | −0.6645 |
| POLB | 688899 \|\| [symbol] | −0.147 | −0.311 | 0.628 | 0.627 | 0.029 | 0.551 | 1.15 | 0 | 0.292 | 0.065 | −0.166 | −0.15 | 0.341 |
| POLR2F | 695452 \|\| [symbol] | 0.7415 | 0.3745 | −0.4355 | 0.1125 | −0.1625 | 0.1715 | −0.1625 | 0.4205 | −0.1945 | −0.1795 | −0.2745 | 0.2955 | 0.0865 |
| POLR2H | 692429 \|\| [symbol] | 0.3705 | −0.0175 | −0.5545 | 0.1725 | −0.2915 | 0.2385 | 0.0775 | −0.0545 | 0.0835 | 0.4265 | 0.8405 | 0.3595 | −0.2805 |
| POMC | 883917 \|\| [symbol] | 0.113 | −0.606 | 0.265 | −0.1 | 0.403 | 0.517 | 1.458 | −0.517 | 0.185 | 0.422 | 0.173 | 0.001 | 0.334 |
| POP4 | 687322 \|\| [symbol] | 0.148 | 0.065 | −0.063 | 0.49 | 0.693 | 0.202 | 0.19 | 0.62 | −0.275 | −0.905 | −0.224 | −0.617 | 0.324 |
| POP7 | 688981 \|\| [symbol] | −0.042 | 0.078 | 0.534 | −0.466 | 0.124 | 0.81 | −0.118 | 0.609 | 0.514 | −0.678 | −0.131 | −0.451 | 0.019 |
| PP1057 | 694894 \|\| [symbol] | −0.0785 | 0.4135 | −0.8525 | 0.1135 | 0.4515 | 0.0615 | 0.6445 | −0.4715 | 1.2805 | 0.4885 | −0.2875 | 0.0615 | −0.0175 |
| PPFIA1 | 684385 \|\| [symbol] | −0.033 | 0.063 | −0.154 | 0.583 | −0.055 | 0.383 | 0.59 | 0.618 | −0.078 | 0.473 | 0.343 | 0.9 | −0.351 |
| PPFIBP2 | 688244 \|\| [symbol] | −0.3265 | −0.3025 | 0.0805 | −0.3445 | −0.0035 | −0.0125 | −0.3875 | −0.4155 | 0.2275 | −0.0475 | −0.2625 | −0.5035 | 0.1725 |
| PPGB | 896508 \|\| [symbol] | 0.4535 | −0.0015 | −0.1855 | 0.4995 | −0.3365 | −0.5475 | 0.9165 | −0.6215 | −0.1505 | 1.8685 | 1.0425 | 1.9175 | 0.2945 |
| PPHLN1 | 881157 \|\| [symbol] | 0.0335 | −0.2825 | 0.0845 | 0.5815 | −0.5625 | −0.2345 | −0.4805 | 0.3155 | 0.0575 | 0.4025 | 0.2435 | 0.5015 | 0.0585 |
| PPIF | 685607 \|\| [symbol] | 0.0815 | −0.1045 | −0.7215 | 0.7825 | −0.4065 | −0.0035 | 0.6055 | −0.5465 | 0.2395 | −0.9315 | 0.0425 | 0.2755 | −0.0245 |
| PPIL1 | 693349 \|\| [symbol] | −0.5505 | 0.6935 | −0.9275 | −0.2625 | −0.2185 | −0.3075 | −0.2595 | 0.0385 | −0.8365 | −0.2775 | 1.2405 | 1.6435 | 1.8865 |
| PPOX | 686128 \|\| [symbol] | −1.343 | 0.195 | −0.175 | −0.077 | 1.058 | 1.572 | −0.574 | −0.484 | 0.81 | −1.769 | −0.595 | −0.246 | −0.56 |
| PPP1R16 [symbol] | 884680 \|\| [symbol] | 0.454 | 0.085 | −0.006 | 0.56 | 0.828 | 0.448 | 0.75 | 0.004 | 0.355 | 0 | −0.001 | 0.449 | 0.547 |
| PPP1R3C | 688073 \|\| [symbol] | 0.291 | −0.788 | −1.053 | 0.02 | −0.21 | −0.174 | 0.809 | −0.774 | −0.15 | −1.427 | −0.101 | 0.623 | −0.201 |
| PPP2R2D | 669931 \|\| [symbol] | −0.9285 | 1.3255 | 0.5405 | −0.4605 | 1.2045 | 0.8835 | −0.3695 | 0.2265 | −0.2485 | 0.8425 | 1.5205 | 4.0025 | −1.1015 |
| PPP2R5A | 886848 \|\| [symbol] | 0.025 | −0.284 | 0.28 | −0.076 | −0.783 | 0.294 | 0.273 | 0.012 | 0.469 | −0.078 | 0.236 | 0.18 | 0.335 |
| PRAME | 690640 \|\| [symbol] | −0.0515 | −0.0465 | −0.3555 | −0.0685 | 0.2885 | 0.8885 | 0.3765 | −0.2575 | −0.0395 | 0.4075 | 0.1815 | −0.0575 | 0.3965 |
| PRC1 | 685404 \|\| [symbol] | 0.59 | 0.589 | 0.246 | 0.004 | −0.004 | −0.329 | −0.01 | 0.261 | −0.051 | −0.458 | −0.07 | −0.445 | −0.43 |
| PRIM2A | 987823 \|\| [symbol] | −0.0705 | −0.3115 | 1.2345 | 0.2435 | 0.0165 | −0.4075 | −0.5055 | −0.2335 | −1.3025 | −0.2735 | 0.7275 | 0.5945 | −0.8715 |
| PRKACB | 698518 \|\| [symbol] | 0.32 | −0.159 | −0.244 | −0.732 | 0.193 | 0.401 | −0.521 | −0.276 | 0.302 | −0.186 | −0.872 | −1.332 | 1.327 |
| PRKCB1 | 894132 \|\| [symbol] | −0.496 | 0.085 | −0.322 | −0.948 | −0.749 | −0.19 | −0.114 | 0.381 | 0.107 | −0.514 | 0.233 | −0.37 | 0.257 |
| PRKCI | 686943 \|\| [symbol] | −0.484 | −0.429 | −0.348 | 0.779 | −0.05 | 0.268 | −0.302 | −0.248 | 0.947 | 0.695 | −0.049 | 0.311 | 1.2 |
| PRKRIR | 684358 \|\| [symbol] | −0.0185 | 0.4155 | −0.4095 | −0.2175 | 0.4825 | 0.9635 | −0.5215 | 0.0205 | 0.3345 | −0.2895 | 0.0025 | −0.3445 | 0.0035 |
| PRKWNK1 | 683385 \|\| [symbol] | 0.2655 | 0.7425 | −1.4835 | −1.3055 | −2.1855 | −0.8705 | −0.4815 | 0.4685 | −0.5885 | 0.1135 | −0.6045 | −1.1085 | −2.5305 |
| PRKX | 808136 \|\| [symbol] | −0.202 | −0.178 | 0.805 | 0.278 | −0.05 | −0.239 | 0.591 | −0.023 | −0.513 | −0.115 | −0.248 | −0.002 | 0.031 |
| PRNP | 692320 \|\| [symbol] | −0.0855 | 0.4075 | −0.3905 | −0.4455 | 0.2055 | 0.4645 | 0.4995 | −0.3375 | −0.7485 | 0.4435 | −0.0015 | −0.2695 | 0.0815 |
| PROL5 | 884331 \|\| [symbol] | −0.4415 | 0.1315 | −1.0645 | −0.2795 | −0.5475 | 0.5275 | 0.5215 | 0.1875 | −0.7265 | −0.4835 | −0.9445 | −0.9075 | 0.1695 |
| PRPF18 | 685943 \|\| [symbol] | −0.8845 | 0.0585 | 0.1835 | −0.5325 | −1.4745 | −0.2825 | −0.4795 | −0.0245 | 0.3035 | −0.0485 | −0.4845 | −0.7945 | 0.3185 |
| PRPSAP1 | 681061 \|\| [symbol] | 0.0735 | 0.8885 | −1.7905 | −0.8375 | −1.5815 | −2.2165 | −2.0135 | −1.0365 | 0.6815 | 1.8185 | −0.2145 | −0.2685 | −2.2376 |
| PRSS12 | 692634 \|\| [symbol] | 0.1876 | −1.4585 | −0.5205 | 0.5625 | 1.5785 | −0.2695 | 0.4825 | 0.1105 | −0.0285 | −0.9165 | −1.2875 | −1.5225 | −0.1845 |
| PRSS16 | 895121 \|\| [symbol] | 0.2465 | 0.3475 | −0.2735 | 0.2275 | 0.6765 | 0.7635 | −0.4785 | −0.1385 | 0.6035 | 0.8045 | 0.3995 | 0.3105 | 0.7535 |
| PSD3 | 688328 \|\| [symbol] | −0.0505 | −0.0655 | −0.0815 | 0.4595 | 0.0915 | 0.8105 | 0.5735 | 0.3425 | 0.0655 | 0.6205 | 0.7555 | 0.3855 | 0.3595 |
| PSMA3 | 682239 \|\| [symbol] | −0.323 | 0.148 | −0.211 | 0.476 | 0.059 | 1.223 | −0.263 | 0.233 | −0.399 | 0.233 | 0.202 | 0.243 | 0.077 |
| PSMA7 | 694665 \|\| [symbol] | −0.335 | 0.701 | −0.611 | 0.284 | 0.312 | 0.728 | −0.897 | 0.264 | 0.229 | 0.486 | 0.588 | 0.557 | 0.071 |
| PSMC5 | 687471 \|\| [symbol] | −0.0285 | −0.4485 | −0.1195 | 0.0455 | 1.0185 | 1.3285 | 0.4475 | 0.2195 | −0.5335 | 0.6935 | −0.7205 | −0.8225 | −0.3175 |
| PSMC6 | 693728 \|\| [symbol] | −0.577 | 0.079 | 0.067 | −0.473 | −0.192 | 0.915 | −0.051 | 0.027 | 0.249 | 0.88 | 0.874 | 0.146 | 0.336 |
| PSMD12 | 697390 \|\| [symbol] | 1.9495 | 3.1405 | 2.4045 | 2.3415 | −0.1695 | 3.5965 | −0.3925 | 3.0855 | −0.3275 | −0.0295 | 1.3465 | 2.4315 | 2.8335 |
| PSMD12 | 697530 \|\| [symbol] | −0.5175 | 1.8355 | −1.6195 | 0.3915 | 0.8445 | 0.2795 | 0.6625 | −0.2305 | 0.1355 | −0.3655 | 1.8295 | 1.3915 | 1.4445 |
| PSMD3 | 681750 \|\| [symbol] | −0.4835 | 0.0615 | −1.4125 | −0.5175 | −1.0855 | −0.3355 | −0.8915 | −0.1545 | 0.1315 | 0.5595 | −0.5895 | −1.1905 | 0.6825 |
| PSPH | 685810 \|\| [symbol] | 1.143 | 0.091 | −0.387 | −1.058 | −1.072 | −0.344 | −0.112 | 0.312 | −0.077 | −0.427 | −0.286 | −0.795 | −0.677 |
| PTD012 | 884258 \|\| [symbol] | −0.272 | 0.537 | −0.247 | −0.091 | 1.101 | −0.204 | −0.258 | 0.593 | 1.959 | −0.376 | −0.275 | −0.188 | −0.228 |
| PTGER3 | 885654 \|\| [symbol] | 0.825 | 0.338 | 0.929 | 0.805 | −0.187 | 0.361 | 0.942 | −0.455 | 0.609 | 0.995 | 0.337 | 1.015 | −0.953 |
| PTHLH | 883001 \|\| [symbol] | 0.3525 | 0.1185 | −1.2615 | −0.2145 | −0.2385 | 0.2005 | −0.3715 | −0.0485 | 0.3415 | 0.0435 | −0.1125 | −0.3745 | −0.0065 |
| PTK6 | 690829 \|\| [symbol] | −0.513 | −0.145 | 0.096 | −0.142 | −0.014 | −0.104 | −0.027 | 0.243 | −0.133 | 0.115 | 0.074 | −0.196 | −0.208 |
| PTPN4 | 693676 \|\| [symbol] | 0.495 | 0.407 | 0.123 | 1.474 | 0.8 | 0.481 | 0.603 | −0.271 | −0.151 | 0.254 | −0.147 | 0.23 | 0.475 |
| PTPN7 | 887885 \|\| [symbol] | 0.145 | −0.125 | 0.122 | 0.317 | −0.009 | −0.26 | −0.346 | 0.17 | 1.579 | −0.196 | −0.078 | 1.586 | −0.592 |
| PTPRF | 884441 \|\| [symbol] | −0.114 | −0.199 | 0.135 | 0.081 | 8.387 | −0.502 | 0.699 | −0.297 | −0.473 | −1.154 | 0.516 | −0.265 | −0.009 |
| PTPRN2 | 885458 \|\| [symbol] | −0.5455 | 0.0355 | −0.5735 | −0.6025 | −0.3325 | 0.1215 | −0.7645 | 0.4305 | 0.1975 | 0.5995 | 0.5185 | 0.3976 | 0.3385 |
| PTPRT | 894229 \|\| [symbol] | 0.0075 | −0.1605 | 0.4015 | −0.0415 | −0.5835 | 0.1385 | 0.4965 | 0.1225 | 0.5605 | 0.3185 | 0.5395 | 0.4825 | 0.3715 |
| PTS | 691288 \|\| [symbol] | −0.455 | 0.767 | −0.342 | 0.293 | −0.551 | 1.005 | 0.578 | 0.053 | 0.554 | 0.153 | 0.483 | 0.585 | 0.277 |
| PTTG1 | 687187 \|\| [symbol] | −0.1485 | −0.4255 | −0.4315 | 0.0235 | 0.5385 | 0.0005 | 0.5875 | −0.8325 | −1.2305 | −0.7505 | −0.5215 | 0.2205 | 0.0585 |
| PURB | 897168 \|\| [symbol] | 0.0155 | −1.4805 | −0.5705 | −0.1265 | 1.0895 | 0.4475 | −0.6115 | −0.3095 | 0.0195 | 0.2455 | 0.3705 | −0.2305 | −0.6125 |
| PXMP2 | 694802 \|\| [symbol] | 0.0615 | 0.5735 | −0.4005 | 0.3775 | −0.0755 | 0.6835 | 1.3635 | 0.2695 | 0.6255 | 1.2805 | 0.7285 | 0.8965 | 0.4035 |
| PYCARD | 896817 \|\| [symbol] | −0.213 | −0.293 | 0.044 | 0.41 | −0.97 | −0.388 | −0.21 | 0.963 | −0.089 | 0.087 | 0.118 | 1.109 | −0.272 |
| PYCR1 | 683284 \|\| [symbol] | 1.072 | 1.268 | 0.114 | 0.95 | 1.596 | 1.782 | 1.132 | 0.587 | 1.33 | 1.085 | 1.005 | 0.893 | 1.395 |
| QDPR | 695556 \|\| [symbol] | 0.3775 | −0.8065 | −0.1095 | −0.9975 | −0.1405 | 0.6405 | −0.4495 | −0.5175 | 0.8125 | −1.8785 | 0.8345 | 1.1365 | −0.3795 |
| QPRT | 881328 \|\| [symbol] | 0.216 | 0.168 | 0.301 | −0.384 | −0.314 | 0.048 | 0.826 | −1.217 | 1.472 | −1.038 | −0.16 | 0.113 | −1.202 |
| QSCN9 | 891881 \|\| [symbol] | 1.5345 | 0.5785 | −0.7575 | 3.0625 | 0.3105 | 1.9365 | 1.0555 | −1.2545 | 1.1075 | 0.1835 | 1.2275 | 1.4415 | 1.1335 |
| RAB11FIP | 897353 \|\| [symbol] | 0.116 | −0.17 | 0.007 | 0.331 | −0.006 | −0.356 | −0.562 | −0.443 | 0.135 | −9.281 | −0.042 | 0.812 | −0.342 |
| RAB11FIP | 602380 \|\| [symbol] | 0.164 | 0.041 | −0.088 | 1.407 | 0.847 | −0.316 | 0.123 | −0.108 | 1.325 | 0.65 | 0.178 | 1.081 | −0.189 |
| RAB13 | 681521 \|\| [symbol] | 0.044 | −0.007 | 0.219 | −0.528 | −0.196 | 0.508 | −0.568 | 0.132 | −0.049 | −0.068 | −0.133 | −0.096 | −0.375 |
| RAB27A | 689787 \|\| [symbol] | −1.091 | −0.829 | 0.753 | −1.344 | −0.006 | −0.642 | −1.631 | 0.011 | −0.705 | −1.861 | 0.001 | −0.66 | 2.124 |
| RAB30 | 690805 \|\| [symbol] | −0.1635 | −0.2755 | −0.1005 | 1.0755 | 0.9595 | 0.5115 | 0.4805 | −0.2515 | 0.1485 | 0.2305 | 0.4515 | 0.5105 | 0.6736 |
| RAB34 | 967074 \|\| [symbol] | −1.414 | −0.438 | 0.104 | −0.106 | 0.214 | −0.7 | −0.358 | −0.453 | 0.318 | −0.406 | 0.205 | 0.125 | −0.505 |
| RAB35 | 869469 \|\| [symbol] | 0.139 | −0.088 | −0.505 | 0.203 | 0.103 | −0.125 | 0.414 | −0.058 | −0.081 | −0.155 | 0.136 | 0.308 | 0.037 |
| RAB39B | 896999 \|\| [symbol] | 1.0775 | −0.8905 | −0.6715 | −0.4965 | 0.9245 | −0.2285 | 0.4295 | 0.0235 | −0.2875 | −0.3205 | 0.8625 | 0.4105 | 0.2535 |
| RAB3A | 686794 \|\| [symbol] | 0.379 | 0.000 | 0.143 | −0.127 | −1.706 | 1.052 | −0.288 | −1.455 | −0.482 | 1.334 | −0.156 | −0.66 | −0.172 |
| RAB3IP | 690420 \|\| [symbol] | 0.127 | −0.33 | −0.158 | −0.3 | −0.624 | 0.22 | −0.508 | −0.383 | −0.327 | −0.38 | −0.512 | −0.284 | −0.805 |
| RABEP1 | 687547 \|\| [symbol] | −0.716 | −0.771 | −0.244 | −0.496 | −0.678 | 0.413 | 0.744 | −0.359 | 0.055 | 0.501 | 0.676 | −0.278 | 0.323 |
| RABGAP1 | 694298 \|\| [symbol] | −0.4415 | −0.1595 | −0.6845 | −0.0925 | −0.8825 | −0.1585 | −0.9915 | 0.1175 | 0.0925 | 0.8225 | −0.5875 | −0.3675 | −0.6135 |
| RACGAP1 | 690792 \|\| [symbol] | −0.6455 | −0.8965 | −0.6255 | 0.3605 | −0.2785 | 0.0855 | 0.2085 | −0.0555 | 0.0385 | 1.3285 | 0.4125 | 0.8615 | 1.5655 |
| RAD17 | 681218 \|\| [symbol] | −1.743 | −0.066 | 0.027 | −0.703 | −0.17 | 0.171 | −0.284 | 0.061 | 0.318 | −0.179 | 0.328 | −0.085 | −0.343 |
| RAD51 | 682122 \|\| [symbol] | −0.082 | 0.185 | 0.177 | 0.092 | 0.401 | 0.126 | 0.213 | 0.126 | 0.372 | 0.103 | −0.336 | 0.137 | −0.03 |
| RAD51AP | 587981 \|\| [symbol] | −0.7985 | 0.0795 | 0.0995 | 0.7485 | 0.5275 | 0.6835 | 0.7915 | 0.4476 | −0.2065 | 0.5935 | 0.2875 | 0.4185 | 1.0225 |
| RAD52 | 603300 \|\| [symbol] | −0.012 | −1.076 | 0.164 | −0.046 | −0.138 | 0.468 | 0.54 | −0.629 | 0.6 | −0.991 | 0.091 | 0.344 | −0.11 |
| RAD54L | 890991 \|\| [symbol] | −0.1635 | −0.0785 | 0.3895 | −0.2445 | 0.1575 | 0.3195 | 0.2185 | 0.1515 | 0.8525 | 0.2735 | 0.4335 | 0.4555 | 1.3985 |
| RAMP | 896334 \|\| [symbol] | 4.7555 | 1.9535 | −0.4315 | 0.6145 | −1.5835 | −0.8515 | 0.0585 | −0.8076 | −1.3715 | 1.6075 | 1.5815 | 2.3825 | −1.4245 |
| RANBP1 | 896056 \|\| [symbol] | −0.6385 | 1.5445 | −0.4205 | −0.1125 | −0.4815 | −0.0245 | 0.0375 | −0.0045 | 0.2125 | −0.7885 | −0.5735 | −0.9705 | −0.0855 |
| RARRES1 | 896860 \|\| [symbol] | −0.183 | −0.805 | −1.278 | −1.273 | −0.198 | −0.98 | −0.946 | −0.818 | 0.395 | −0.35 | −0.962 | −0.216 | −1.201 |
| RBBP7 | 695677 \|\| [symbol] | −0.1385 | 0.0695 | −0.2585 | −0.1885 | 0.4475 | 0.2605 | 0.7035 | −0.2065 | 0.1845 | −1.0655 | 0.5276 | 0.3155 | 0.4095 |
| RBBP8 | 690099 \|\| [symbol] | −0.0025 | −0.1835 | 0.2025 | −0.3475 | 0.8525 | −0.2265 | −0.3025 | 0.1895 | −0.3545 | 0.7225 | −0.0605 | 0.1735 | −0.7825 |
| RBM8A | 881239 \|\| [symbol] | 2.219 | −0.398 | 0.108 | 1.721 | −0.892 | −0.694 | −0.844 | 0.262 | 0.28 | 3.582 | 2.227 | 3.516 | 1.794 |
| RBX1 | 885240 \|\| [symbol] | −0.63 | 0.221 | −0.261 | 1.42 | −0.327 | 0.162 | −0.622 | 0.549 | 0.443 | 0.547 | −0.084 | 0.103 | −0.541 |
| RDHE2 | 695026 \|\| [symbol] | 0.034 | −0.313 | 0.081 | −0.847 | −0.411 | −0.518 | −0.47 | −0.012 | 0.057 | 0.292 | −0.312 | −0.27 | −0.322 |
| REA | 691804 \|\| [symbol] | −0.312 | −0.149 | 0.458 | 0.388 | 0.228 | 0.167 | 0.158 | −0.101 | −0.17 | 0.073 | 0.229 | −0.028 | 0.009 |
| REC14 | 685901 \|\| [symbol] | 0.5535 | 0.0045 | 0.1795 | −0.2895 | −0.4755 | −0.1225 | 0.0735 | 0.1945 | 0.7055 | −0.5675 | −0.2405 | 0.2885 | 0.0905 |
| REG1A | 895196 \|\| [symbol] | −0.185 | −0.831 | 0.472 | 0.275 | 0.57 | 0.597 | 0.525 | −0.14 | 0.277 | −0.094 | 0.148 | 0.086 | −0.277 |
| RELB | 684812 \|\| [symbol] | 0.548 | 0.139 | −0.489 | −0.009 | 0.205 | 0.125 | 0.187 | −0.285 | 2.812 | −0.435 | −0.374 | −0.121 | −0.247 |
| REPIN1 | 683068 \|\| [symbol] | −0.509 | −0.605 | −0.227 | −0.729 | −0.858 | −1.096 | −0.902 | −0.445 | 0.484 | −1.016 | −0.27 | −0.288 | −0.728 |
| REPS2 | 887793 \|\| [symbol] | −0.1215 | −0.0255 | 0.0545 | 0.1525 | −0.3035 | 0.2615 | −0.3405 | −0.0975 | 0.0885 | 0.6635 | 0.1295 | 0.0035 | 0.0165 |
| RERG | 687590 \|\| [symbol] | −0.5725 | −0.4335 | −0.1925 | −0.2175 | 0.0435 | −0.4275 | 0.6155 | −0.4076 | 0.2715 | −0.2915 | 0.0035 | −1.1075 | −0.6405 |
| RFC4 | 684079 \|\| [symbol] | 1.3935 | 1.3145 | 0.4105 | −0.3635 | 0.5825 | −0.0045 | 2.0745 | −0.3255 | −0.7415 | 0.3155 | 0.1355 | 0.0605 | 0.0175 |
| RGS10 | 695917 \|\| [symbol] | −0.0465 | −0.2475 | −0.0815 | 1.1485 | 1.0745 | 0.1005 | 0.6765 | 0.1705 | −0.0105 | 1.5315 | 0.6135 | 0.3465 | 0.0075 |
| RH8DL2 | 984395 \|\| [symbol] | −0.899 | −0.065 | 0.465 | 0.268 | −1.968 | 0.338 | 0.325 | −0.009 | 0.013 | −0.183 | −0.152 | −0.331 | −0.681 |
| RIBC2 | 896320 \|\| [symbol] | −0.271 | −0.45 | 0.604 | −0.164 | 0.372 | 0.731 | 1.142 | −0.025 | −0.014 | −0.089 | 1.015 | 0.639 | 0.286 |
| RIC-8 | 590624 \|\| [symbol] | −1.04 | −0.42 | −0.6 | −0.83 | −0.899 | −0.585 | −0.374 | −0.48 | −0.567 | −0.592 | −0.556 | 0.73 | −0.637 |
| RIT1 | 693292 \|\| [symbol] | 0.279 | 0.062 | −0.329 | 0.403 | 0.919 | 0 | 0.278 | 0.26 | 0.338 | −0.442 | −0.313 | −0.106 | 0.007 |
| RLN2 | 688741 \|\| [symbol] | 1.581 | −0.391 | −0.438 | −0.835 | 2.406 | 0.6 | −0.017 | 0.069 | 0.266 | 0.107 | 0.122 | −0.155 | −0.785 |
| RNF10 | 696512 \|\| [symbol] | 0.1346 | 0.5895 | 0.0015 | −0.2125 | −0.0025 | 0.1695 | 0.2225 | −0.1085 | 0.3265 | 0.6715 | −0.1255 | −0.2335 | −1.1865 |
| RNF135 | 891324 \|\| [symbol] | 0.5915 | −0.0965 | −0.3485 | 0.4105 | −0.0135 | −0.3335 | −0.4175 | −0.4455 | 0.0185 | −0.5445 | 0.0815 | −0.0195 | −0.2995 |
| RNF7 | 981076 \|\| [symbol] | −0.010 | −0.305 | −0.383 | 0.588 | −0.153 | 0.311 | 0.64 | −0.416 | −0.388 | 0.015 | 0.411 | 0.804 | −0.051 |
| ROPN1 | 692734 \|\| [symbol] | 0.8075 | 3.1995 | 1.4485 | −0.5585 | 0.1565 | −0.8185 | −0.9205 | −0.3415 | −2.0685 | −1.0175 | 2.9085 | 3.9335 | 0.2515 |
| RPA3 | 684912 \|\| [symbol] | −0.0605 | 0.5585 | −0.1655 | 1.0005 | −1.3925 | −0.2595 | −0.2365 | 0.1665 | −0.1325 | 0.0085 | 0.3055 | 0.0195 | −0.1335 |
| RPESP | 690226 \|\| [symbol] | 0.091 | 0.535 | −0.311 | 0.276 | −0.432 | 0.089 | −0.549 | −0.156 | −0.628 | −0.393 | 0.746 | 0.196 | 1.607 |
| RPL11 | 684149 \|\| [symbol] | 0.07 | 0.332 | 1.866 | 0.427 | −0.693 | −0.434 | −0.63 | 0 | 0.47 | 0.149 | 0.585 | −0.002 | 0.465 |
| RPL13 | 690460 \|\| [symbol] | −0.5 | 0.576 | 0.133 | 0.211 | −1.005 | 0.534 | −0.624 | 0.02 | −1.248 | 0.251 | −0.539 | −1.097 | −1.008 |
| RPL13A | 687951 \|\| [symbol] | 0.3335 | 0.8845 | −0.0685 | 0.5175 | 0.0965 | 0.0145 | −0.5175 | 0.6215 | −0.0555 | 1.1905 | 1.3535 | 0.9095 | 0.3015 |
| RPL15 | 682985 \|\| [symbol] | −0.732 | 0.041 | −0.936 | −0.333 | 0.572 | −0.63 | −0.511 | 0.043 | −0.501 | 0.849 | 0.838 | 0.381 | −0.687 |
| RPL18A | 883895 \|\| [symbol] | −0.038 | 0.363 | −0.621 | −0.667 | −0.13 | −0.223 | −0.816 | 0.039 | −0.712 | 0.203 | 0.534 | −0.293 | 0.017 |
| RPL19 | 888555 \|\| [symbol] | −0.016 | −0.755 | 0.311 | 1.442 | 0.837 | −0.636 | −0.804 | −0.133 | 0.041 | 0.157 | 0.677 | 0.308 | 1.343 |
| RPL21 | 092099 \|\| [symbol] | 0.0085 | −0.6815 | 0.4055 | 1.3125 | 0.6965 | −0.5025 | −0.7875 | 0.1025 | 0.0935 | 0.0345 | 0.4485 | 0.0395 | 1.1555 |
| RPL23A | 481589 \|\| [symbol] | −0.429 | 0.45 | −0.594 | −0.202 | −1.751 | −0.183 | −0.866 | 0.370 | −0.051 | 0.099 | 0.322 | −0.77 | −0.718 |
| RPL23A | 692544 \|\| [symbol] | −0.4255 | 0.5205 | −0.2785 | 1.1335 | −1.3275 | −0.1405 | −0.5895 | 0.1995 | −0.0475 | 0.7435 | 0.2205 | 0.2715 | −0.3845 |
| RPL26L1 | 685938 \|\| [symbol] | −0.0455 | −0.1255 | −0.1745 | 1.2015 | −0.5925 | 0.0295 | −0.0495 | 0.3875 | 0.5525 | 0.5155 | 0.5265 | 0.1325 | 0.9985 |
| RPL29 | 690810 \|\| [symbol] | −0.0935 | −0.1475 | −0.1005 | 0.9355 | −0.5205 | 0.0915 | −0.7275 | 0.2625 | 0.1776 | 0.3045 | 0.4215 | 0.5535 | −0.4575 |
| RPL35 | 687324 \|\| [symbol] | −0.2585 | 0.1805 | 1.1895 | −0.8705 | −1.2095 | −0.3915 | −0.9455 | 0.0845 | 0.1476 | 0.1125 | 0.2535 | −0.5295 | 0.5795 |
| RPL36AL | 693407 \|\| [symbol] | −0.4335 | −0.0105 | 0.5555 | 0.2395 | 0.1605 | −0.1875 | 0.0705 | −0.0035 | 0.0275 | 0.1855 | 0.7875 | 0.0505 | −0.0245 |
| RPS16 | 687942 \|\| [symbol] | −0.5765 | −0.0035 | −0.6985 | −0.8135 | −0.7525 | −0.5015 | −0.4545 | 0.2995 | 0.0295 | −0.3365 | 0.1165 | −0.2205 | −0.8345 |
| RPS4Y1 | 683806 \|\| [symbol] | 0.8125 | 0.7705 | 0.0495 | 0.3055 | −0.5565 | −0.0475 | 1.2185 | −0.2755 | −0.5025 | 1.8755 | 0.3735 | 0.2615 | −0.5725 |
| RRAGA | 688744 \|\| [symbol] | −0.733 | −0.563 | 0.407 | −0.14 | −0.743 | −0.065 | 0.164 | −0.438 | −0.061 | −0.001 | 0.076 | 0.055 | 0.215 |
| RRAGD | 891801 \|\| [symbol] | −0.565 | 0.356 | 0.006 | 1.562 | 0.513 | 1.406 | 1.66 | 0.585 | 0.21 | 0.902 | 0.996 | 0.155 | 1.023 |
| RRM1 | 884258 \|\| [symbol] | −0.128 | −0.274 | −0.178 | −1.138 | −0.345 | 0.222 | 0.624 | −0.578 | 0.002 | −0.045 | 0.134 | 0.07 | 0.624 |
| RRM2 | 694262 \|\| [symbol] | 0.264 | 0.598 | −0.278 | −1.173 | −2.826 | −1.944 | −1.618 | −0.183 | −1.108 | −1.85 | −0.318 | −0.446 | −2.004 |
| RSAFD1 | 687475 \|\| [symbol] | −0.0775 | 0.0585 | 0.2155 | 0.0955 | 0.0175 | −0.0425 | 8.0715 | 0.5835 | −0.0665 | 0.5155 | 0.0655 | 0.1255 | −0.8635 |
| RSNL2 | 682893 \|\| [symbol] | 0.3795 | 0.4395 | −0.4885 | −0.4315 | 0.1885 | 0.1815 | −0.1855 | 0.0755 | −0.5305 | 0.6785 | 0.8465 | 1.1145 | 0.8205 |
| RSU1 | 682633 \|\| [symbol] | 0.232 | 0.427 | 0.683 | 0.185 | 0.312 | 0.804 | 1.425 | 0.72 | 0.837 | −0.394 | −0.349 | −0.173 | −0.068 |
| RTN4IP1 | 698006 \|\| [symbol] | −0.118 | 0.584 | −0.781 | −3.141 | 0.862 | 1.269 | −0.516 | 0.615 | 1.99 | −0.118 | 0.813 | 0.84 | 2.158 |
| S100A11 | 688967 \|\| [symbol] | −0.045 | 0.572 | −1.098 | −1.177 | 1.165 | 1.112 | −0.76 | 1.127 | 1.465 | −1.106 | 0.588 | 0.717 | 0.66 |
| S100A14 | 689448 \|\| [symbol] | 4.6545 | 2.2655 | −0.0375 | −1.8505 | 3.9905 | −0.1525 | 4.2675 | 0.1345 | −1.5505 | 2.1545 | 2.8125 | 2.8405 | 4.7405 |
| S100A16 | 682683 \|\| [symbol] | 0.0415 | 0.2115 | 0.4975 | 0.4885 | −0.5505 | 0.3715 | 0.0075 | 0.4735 | 0.1845 | 0.1655 | −0.0415 | 0.0795 | 0.6715 |
| S100A9 | 681875 \|\| [symbol] | −0.854 | −0.474 | −0.451 | −0.391 | −0.944 | −0.274 | −1.049 | −0.275 | −0.067 | −1.912 | 0.02 | 0.647 | −0.322 |
| SAE1 | 695822 \|\| [symbol] | 0.0555 | −0.2695 | 0.1375 | 0.8855 | −0.0835 | 0.3785 | 0.2175 | 0.2815 | 1.3005 | 0.2485 | 0.6355 | 1.0385 | 0.6035 |
| SALL2 | 698484 \|\| [symbol] | −0.014 | 0.291 | 0.222 | 0.749 | 1.244 | −0.855 | 0.04 | 0.417 | 0.644 | 0.314 | 0.152 | 0.833 | 0.39 |
| SARA1 | 690066 \|\| [symbol] | −0.4555 | 0.4225 | 0.5025 | 0.2735 | −0.4705 | 0.3785 | −0.3735 | 0.1855 | −0.0685 | −0.2595 | −0.2475 | −0.3805 | −0.1655 |
| SARA2 | 888945 \|\| [symbol] | −0.309 | 1.039 | 0.685 | 0.18 | 0.015 | 0.572 | −0.34 | 0.611 | 0.087 | 1.805 | 0.928 | 0.84 | −0.403 |
| SAS | 897574 \|\| [symbol] | −0.227 | −0.128 | −0.354 | 0.362 | −1.248 | −0.168 | 0.368 | 0.279 | −0.051 | −0.557 | −0.082 | 0.391 | −1.235 |
| SCIN | 696329 \|\| [symbol] | 0.6845 | 0.8895 | −0.2295 | −0.7145 | −0.9055 | 0.1005 | 8.4805 | 0.1755 | −0.7425 | 0.6955 | −0.0145 | −0.3405 | −0.0675 |
| SCML1 | 683929 \|\| [symbol] | −0.124 | 0.594 | −1.102 | 0.088 | −0.248 | 0.39 | −0.542 | 0.523 | −0.852 | 0.482 | 0.734 | 1.016 | 0.804 |
| SCPEP1 | 889318 \|\| [symbol] | 1.2005 | 1.5205 | 0.8645 | 0.1585 | 1.0125 | 1.5475 | 1.7755 | 1.3665 | 1.1225 | 1.6215 | 1.7625 | 0.6695 | 1.4125 |
| SCRIB | 887588 \|\| [symbol] | 0.721 | 1.517 | 0.519 | 0.573 | 0.808 | 0.036 | 0.091 | 0.182 | 0.118 | −0.167 | 0.163 | 0.774 | 0.003 |
| SDC1 | 881258 \|\| [symbol] | 0.189 | 0.151 | −0.724 | −0.706 | −0.708 | 0.303 | −0.091 | −0.049 | −0.009 | 0.429 | 0.184 | −0.318 | −0.416 |
| SDC3 | 893185 \|\| [symbol] | 0.5685 | 0.8135 | 0.1005 | 0.8955 | −0.0425 | 0.0745 | 0.6985 | −0.0835 | 0.4585 | 1.0015 | 0.4645 | 0.5525 | 0.5515 |
| SDCCAG0 | 890950 \|\| [symbol] | 0.4875 | 0.9615 | 0.0225 | 0.4695 | −0.5855 | −0.1745 | 0.7375 | −0.0265 | −0.5855 | 0.4945 | −0.0875 | −0.1545 | 0.7975 |
| SEC61G | 693650 \|\| [symbol] | −0.502 | 0.074 | −0.183 | 0.095 | −0.431 | 0.73 | −0.182 | −0.389 | 0.097 | −0.047 | 0.026 | 0.671 | −0.467 |
| SECP43 | 894767 \|\| [symbol] | −0.0575 | −0.6595 | −0.2305 | 0.5905 | −0.8695 | −1.2125 | 0.0715 | −0.2435 | 0.1825 | −1.3755 | −1.0175 | −0.1825 | −0.2605 |
| SEDLP | 693304 \|\| [symbol] | 0.229 | 0.026 | −0.776 | −2.005 | 0.64 | −0.606 | 0.018 | −0.264 | 1.161 | −1.678 | −0.657 | −1.906 | −1.804 |
| SEMA3B | 696022 \|\| [symbol] | 0.8775 | 0.4615 | 0.1715 | 1.3045 | −0.7785 | 0.2105 | 0.9465 | 0.4525 | 0.3295 | −0.1325 | −0.3335 | 0.5735 | 0.4035 |
| SEMA3C | 601813 \|\| [symbol] | 0.4255 | 0.6885 | −1.5615 | 0.4425 | 0.1965 | −0.4005 | −0.1535 | −0.4645 | 0.0185 | 0.3145 | −0.5055 | −0.3315 | −0.1535 |
| SEPW1 | 683289 \|\| [symbol] | −0.455 | −0.474 | −0.933 | −0.852 | −0.273 | −1.287 | −1.448 | −0.611 | 0.301 | −0.409 | −0.862 | 0.293 | −1.085 |
| SFRP1 | 690913 \|\| [symbol] | −0.9925 | −0.3255 | 0.2175 | −1.1665 | −0.1985 | −0.8595 | −0.2535 | −0.0425 | −0.2555 | −0.8955 | 0.8255 | −0.0735 | −0.3035 |
| SERPINA [symbol] | 892607 \|\| [symbol] | 1.412 | 0.43 | −1.635 | −0.987 | −3.053 | −1.067 | −2.287 | −1.04 | −1.3 | −1.907 | −0.88 | −0.316 | −1.397 |
| SERPINB [symbol] | 683845 \|\| [symbol] | 0.679 | −0.437 | −0.719 | 1.593 | 0.492 | 0.799 | −0.985 | 1 | −0.098 | 0.289 | −0.364 | −0.093 | −0.103 |
| SFRP1 | 890138 \|\| [symbol] | −0.373 | 0.928 | −1.699 | −0.051 | −0.078 | 0.366 | 0.046 | 0.966 | 1.023 | −0.393 | −0.218 | −0.68 | −0.82 |
| SFRS6 | 689301 \|\| [symbol] | −0.1625 | 0.8985 | −0.2005 | −1.5645 | −1.0135 | −0.3915 | −1.6315 | 0.7835 | −0.1595 | −1.0955 | 0.7285 | 0.2735 | −1.7025 |
| SGCD | 694781 \|\| [symbol] | −0.3118 | −0.2785 | −0.3075 | −0.4205 | 0.8025 | 1.2085 | −0.7225 | 0.2055 | 1.1645 | −0.1545 | −0.3155 | −0.6845 | −2.2385 |
| SGCE | 585105 \|\| [symbol] | 0.001 | −0.271 | 0.143 | −0.594 | 0.044 | 0.316 | 0.755 | −0.406 | 0.107 | 0.535 | 0.114 | −0.177 | 0.434 |
| SH3BGRL | 694455 \|\| [symbol] | −1.165 | −1.321 | −0.578 | 0.933 | −0.565 | 1.187 | 0.049 | −0.118 | 0.333 | −2.058 | −0.493 | 0.74 | −0.908 |
| SHFM1 | 583462 \|\| [symbol] | 0.0375 | −0.1885 | −0.9825 | 0.9615 | 0.2115 | 0.1805 | 0.0195 | 0.0665 | 0.0785 | 0.2995 | 0.2305 | 0.7665 | 0.4505 |
| SIAT7B | 685595 \|\| [symbol] | 0.101 | −0.123 | 0.152 | 0.258 | −0.533 | 0.104 | 0.083 | −0.053 | −0.776 | 0.566 | 0.165 | 0.654 | −0.071 |
| SIGLEC12 | 680532 \|\| [symbol] | 0.064 | −0.444 | −0.254 | 0.778 | −0.092 | 0.004 | −0.378 | −0.198 | 0.388 | 0.694 | 0.349 | 0.722 | 0.191 |
| SIT | 682124 \|\| [symbol] | −0.4805 | −0.0375 | 0.3205 | −0.3025 | 0.0545 | −0.4335 | −0.4315 | −0.1845 | −0.1475 | −1.1355 | −0.1225 | 0.2225 | −0.1715 |
| SITPEC | 681337 \|\| [symbol] | 0.5115 | −0.2955 | −0.0275 | −0.4765 | −0.1275 | −0.0255 | 0.9015 | −0.2785 | 0.2175 | −0.3115 | −0.5135 | 0.0025 | 0.1785 |
| SKP1A | 684965 \|\| [symbol] | 0.0715 | −0.3965 | −1.4065 | 2.7885 | −0.9215 | 0.2745 | 2.6035 | 0.0055 | 1.3485 | −0.5035 | −0.1365 | −1.4695 | 2.5495 |
| SLC12A9 | 696704 \|\| [symbol] | −0.838 | −1.002 | 0.988 | −1.743 | −1.383 | −0.653 | −0.702 | −0.022 | 0.27 | −1.166 | −1.133 | 0.54 | −1.625 |
| SLC13A3 | 689746 \|\| [symbol] | −0.3295 | −0.6725 | −0.9555 | −0.2445 | 0.2495 | −0.5665 | 2.4055 | −0.1735 | 1.1365 | −1.0435 | −0.8505 | 0.1565 | −1.3815 |
| SLC16A6 | 687056 \|\| [symbol] | 0.882 | 0.288 | 0.176 | 0.629 | 1.938 | −0.598 | 2.698 | −0.525 | −0.603 | −1.689 | 1.681 | 2.575 | 1.85 |
| SLC19A2 | 603146 \|\| [symbol] | −0.7255 | −1.9595 | 0.1605 | 0.8045 | −0.8485 | −1.0995 | −0.3355 | −0.4215 | 0.4485 | 1.7575 | −0.7085 | −0.2685 | −0.9935 |
| SLC1A1 | 692391 \|\| [symbol] | −1.9105 | −0.6005 | 0.8165 | −1.5795 | −1.9725 | −1.1795 | 0.1955 | 1.2425 | −0.2375 | −1.6565 | −0.4585 | −1.8395 | −0.5535 |
| SLC1A2 | 690726 \|\| [symbol] | −0.0185 | 0.4315 | −0.8935 | 0.3915 | 0.2085 | 0.0615 | 0.3065 | −0.2035 | 0.1215 | −0.9125 | −0.2875 | −0.6145 | 1.1515 |
| SLC24A3 | 680570 \|\| [symbol] | 0.295 | −0.464 | −0.013 | 0.589 | 0.498 | 0.51 | 0.024 | −0.33 | −0.277 | 0.408 | −1.146 | −0.63 | 0.45 |
| SLC25A29 | 692129 \|\| [symbol] | 0.144 | 0.256 | 0.371 | −0.295 | 0.094 | 0.279 | 0.734 | 0.369 | 0.235 | 0.73 | 0.928 | 0.585 | −0.455 |
| SLC25A4 | 684980 \|\| [symbol] | 0.617 | −1.022 | −0.239 | −1.625 | −0.549 | −1.255 | −1.218 | −0.718 | −0.599 | −0.06 | −0.177 | 2.505 | −1.095 |
| SLC25A5 | 681570 \|\| [symbol] | −0.3305 | −0.3205 | 0.0325 | −0.5605 | −0.8605 | 1.0765 | 0.3995 | −0.5785 | −0.0875 | 1.8155 | 0.1265 | 0.3655 | −0.9485 |
| SLC27A2 | 686936 \|\| [symbol] | −0.631 | −0.166 | 0.44 | 0.042 | 0.111 | 1.786 | −0.141 | 0.123 | −0.292 | −0.545 | 0.233 | −0.403 | 0.109 |
| SLC27A5 | 892268 \|\| [symbol] | 0.313 | 0.593 | 0.344 | 0.148 | −1.306 | −0.212 | −0.061 | 0.639 | −0.471 | −0.008 | 1.616 | 1.914 | 0.767 |
| SLC30A8 | 695934 \|\| [symbol] | −0.2385 | −1.3815 | −1.5545 | −3.6195 | 0.6065 | −0.8515 | −1.8075 | −0.3815 | 0.2945 | −1.7545 | 0.5365 | −2.1175 | −2.1885 |
| SLC39A4 | 684775 \|\| [symbol] | 0.955 | 0.586 | −0.057 | −1.58 | −0.631 | −0.418 | 0.356 | −1.172 | −1.27 | −0.022 | 0.905 | −0.56 | −1.009 |
| SLC39A6 | 690413 \|\| [symbol] | 0.2445 | 1.0785 | −0.0005 | −0.0105 | 0.0965 | −0.0805 | 0.7555 | −0.3055 | −0.0745 | −0.1035 | −0.2185 | −0.1605 | −0.3115 |
| SLC40A1 | 695681 \|\| [symbol] | 0.5125 | 0.1985 | 0.2435 | 0.7725 | 1.0555 | 1.0845 | 1.3375 | −0.3205 | 0.0185 | 0.2755 | 0.8955 | 1.0725 | 0.7495 |
| SLC41A3 | 682343 \|\| [symbol] | −0.568 | −0.229 | 0.27 | 2.27 | −0.181 | −0.058 | −0.218 | −0.331 | −0.188 | 1.277 | 0.391 | −0.64 | 0.137 |
| SLC5A6 | 88366 \|\| [symbol] | −0.8145 | −0.3095 | −0.5475 | 0.5755 | 0.6955 | 1.6215 | 0.5515 | 0.7185 | −0.5305 | 0.3025 | −0.0685 | 1.1315 | 3.1815 |
| SLC6A4 | 691977 \|\| [symbol] | −1.3965 | 0.9595 | 0.6985 | −2.5365 | −1.0235 | −1.9985 | −3.7105 | −1.0625 | 5.6475 | −0.9505 | −0.2165 | 1.0685 | −3.0776 |
| SLC9A3R1 | 682747 \|\| [symbol] | −0.8455 | 0.9145 | 0.1415 | −1.2015 | 2.7135 | 1.4805 | 0.3905 | 1.2825 | 0.5485 | 0.5175 | −0.2215 | −0.4105 | 0.8465 |
| SLITRK6 | 685643 \|\| [symbol] | −0.265 | 0.089 | 0.038 | −0.051 | 0.235 | 0.654 | −0.452 | 0.006 | 0.173 | 0.56 | 0.391 | 0.083 | −0.104 |
| SMA3 | 687200 \|\| [symbol] | −0.486 | 0.771 | −0.612 | −0.377 | −0.551 | 0.702 | −0.595 | −0.067 | 0.024 | −1.466 | −0.02 | −0.532 | −0.899 |
| SMAD2 | 696467 \|\| [symbol] | −0.257 | −0.176 | −0.32 | −0.267 | −0.438 | −0.356 | 0.636 | −0.132 | 0.089 | −0.393 | 0.731 | 0.511 | 0.394 |
| SMO | 688958 \|\| [symbol] | −0.346 | −0.082 | 0.355 | 0.355 | 0.782 | −0.045 | 0.007 | −0.309 | 0.025 | −0.505 | 0.048 | 0.388 | 0.415 |
| SMU1 | 589287 \|\| [symbol] | −0.32 | −0.345 | −0.03 | −0.517 | 0.014 | 0.813 | 0.327 | 0.352 | 0.174 | 0.368 | 0.319 | 0.374 | 0.39 |
| SNPH | 584407 \|\| [symbol] | −0.145 | −0.358 | −0.394 | 0.012 | 0.209 | 0.362 | −0.315 | 0.155 | 0.499 | −0.139 | 0.579 | 0.873 | 0.78 |
| SNRPB2 | 698838 \|\| [symbol] | −0.4315 | −0.0035 | 0.1425 | 1.3335 | 0.5505 | 0.0176 | 0.3445 | −0.1525 | 0.7745 | 0.0725 | 0.3695 | 0.7255 | 2.1465 |
| SNRPD1 | 895576 \|\| [symbol] | −0.161 | −0.341 | 0.055 | −0.195 | 0.87 | 1.073 | 0.495 | 0.095 | −0.208 | 0.067 | 0.386 | 0.238 | −0.236 |
| SNRPD3 | 592082 \|\| [symbol] | −0.1555 | 0.2145 | −0.0025 | −1.6355 | −1.1155 | −0.4815 | −1.5105 | 0.1385 | −0.1885 | −1.4845 | −0.5985 | −1.0305 | −0.4185 |
| SNRPG | 684507 \|\| [symbol] | 0.7915 | 0.7055 | −0.2275 | −0.7455 | −0.6655 | 0.0305 | −0.0115 | 0.0115 | −0.1205 | 1.0355 | 1.5955 | 1.3665 | 0.8595 |
| SNX7 | 685968 \|\| [symbol] | −0.8845 | −0.3265 | −0.8685 | 0.0555 | 0.0475 | −0.1565 | −0.2985 | 0.0525 | 0.2845 | 0.2145 | 0.0435 | −0.0745 | −0.1265 |
| SO02 | 684100 \|\| [symbol] | −0.4155 | 0.8805 | −0.3515 | 0.9995 | 0.7365 | 1.2735 | −0.1375 | 0.3975 | 0.2565 | −1.1195 | 0.4835 | 0.0085 | 1.4555 |
| SORCS1 | 690320 \|\| [symbol] | −0.008 | 0.231 | 0.023 | 0.481 | 0.82 | 0.312 | −0.939 | −0.374 | 0.068 | −0.997 | 0.008 | 0.05 | −0.977 |
| SOX4 | 686301 \|\| [symbol] | −0.4325 | 0.6875 | −1.6775 | 0.8975 | −2.5275 | 1.7555 | −1.6415 | −0.5225 | 0.0285 | −2.1545 | −0.4555 | 0.5395 | −1.5155 |
| SOX9 | 661418 \|\| [symbol] | 0.395 | −1.044 | 0.331 | 1.215 | 0.37 | 0.112 | 0.799 | −0.879 | 0.461 | 0.326 | 0.767 | 0.205 | 3.284 |
| SPAG18 | 687462 \|\| [symbol] | 0.0325 | 0.1835 | −0.4805 | −1.0475 | −0.4825 | −0.0885 | −1.0265 | −0.1585 | −0.2975 | 0.6465 | 0.4165 | −0.1695 | 0.5175 |
| SPAG5 | 686120 \|\| [symbol] | 0.0305 | −0.4715 | −0.9045 | 0.5605 | 0.2985 | 0.0975 | −0.1235 | −0.6135 | 0.2275 | 0.1245 | −0.3445 | −0.7745 | −0.5405 |
| SPC18 | 695899 \|\| [symbol] | 0.344 | −0.211 | 0.013 | −0.852 | −0.146 | 0.527 | 0.239 | 0.28 | −0.212 | −0.637 | −0.422 | −1.169 | −0.87 |
| SPFH2 | 688593 \|\| [symbol] | −0.081 | −0.139 | 0.588 | −0.138 | 0.038 | 0.13 | 0.006 | 0.04 | 0.188 | 0.404 | −0.143 | −0.157 | 0.499 |
| SPG3A | 685052 \|\| [symbol] | 0.375 | 1.414 | 1.129 | −1.52 | −0.375 | −0.395 | 0.965 | 0.795 | 0.485 | 0.835 | 1.425 | 2.013 | 1.345 |
| SPPL3 | 884761 \|\| [symbol] | 0.39 | −0.16 | 1.151 | 0.546 | 0.606 | −0.695 | 0.525 | −0.288 | −0.15 | 1.489 | 0.524 | 0.228 | 0.453 |
| SQLE | 765400 \|\| [symbol] | −0.125 | 0.717 | 1.348 | 1.335 | 1.246 | −0.027 | 0.505 | 0.351 | −0.238 | 1.005 | −0.107 | −0.342 | 0.325 |
| SQRDL | 693902 \|\| [symbol] | −0.2835 | 0.6205 | 0.1235 | −0.5275 | 0.0165 | −0.7855 | −0.7515 | −0.1765 | −0.4965 | 0.2335 | −0.5535 | −1.1285 | −1.4375 |
| SREBF1 | 597880 \|\| [symbol] | 0.1915 | 0.2345 | 0.6515 | 0.5505 | −0.3195 | 0.3365 | 0.8015 | 0.2325 | 0.0605 | 0.6855 | 0.4495 | 0.1715 | 0.5095 |
| SRI | 696971 \|\| [symbol] | 0.2135 | 0.0135 | 0.4955 | −0.2915 | 1.5355 | −0.2455 | 0.4315 | 0.0365 | 1.2265 | −0.7475 | −0.9685 | −1.2535 | −0.1105 |
| SRPK1 | 697017 \|\| [symbol] | 0.0715 | 0.0505 | −0.2205 | 1.1565 | 0.2295 | −0.1625 | 0.0445 | 0.1335 | 0.1495 | 0.1415 | −0.0435 | 0.0495 | −0.4285 |
| SSFA2 | 692106 \|\| [symbol] | −0.2365 | 0.7005 | 0.6475 | −0.5715 | 0.3415 | 0.1705 | 0.3335 | 0.5515 | 0.0805 | 0.1925 | 0.3505 | −0.1255 | −0.2645 |
| SSR4 | 686717 \|\| [symbol] | 0.3695 | 0.2545 | −0.5305 | −0.4885 | −0.2605 | 0.1665 | −0.5395 | −0.7065 | −0.5395 | −0.2065 | 0.1375 | −0.0065 | −0.2685 |
| STARD3NI | 686038 \|\| [symbol] | −1.2535 | 0.7565 | 0.8915 | −0.2325 | −0.5605 | −0.4205 | −0.0875 | 1.1835 | 1.4155 | −0.0275 | 0.7755 | 1.4845 | −0.6245 |
| STARD7 | 681306 \|\| [symbol] | −1.4935 | −0.1855 | −0.9455 | −1.5375 | −1.7125 | −1.6065 | −1.4515 | −1.2815 | −0.9395 | 0.5795 | −0.4655 | 0.6855 | −1.1565 |
| STAT1 | 681492 \|\| [symbol] | −0.1325 | 0.4915 | 0.4435 | −1.2465 | 0.1225 | −0.2955 | −0.1835 | −0.0325 | −0.1445 | −0.6695 | −0.3415 | −0.2775 | 0.2565 |
| STC2 | 889625 \|\| [symbol] | −0.3485 | −0.0875 | −0.1485 | −0.1395 | −0.4075 | 0.4115 | 0.4915 | 0.0565 | 0.0015 | 0.5945 | 0.9905 | 0.3935 | 0.9285 |
| STK24 | 888430 \|\| [symbol] | −0.6895 | 0.5955 | 0.0885 | −0.2545 | 1.0875 | 0.1355 | 0.7795 | 0.2005 | −0.9945 | −0.6715 | 0.1315 | −0.3905 | 0.0995 |
| STK6 | 685902 \|\| [symbol] | 0.185 | 0.884 | 0.607 | 1.482 | 1.131 | 1.457 | 1.53 | 2.69 | 1.089 | 0.076 | 0.575 | 0.631 | 0.682 |
| STMN1 | 882764 \|\| [symbol] | 0.3205 | 0.8045 | 1.6575 | 0.3915 | 1.7495 | 0.6595 | 0.3255 | 0.0685 | 0.6855 | −0.1405 | 0.3435 | 0.4765 | 0.6875 |
| STX1A | 691816 \|\| [symbol] | 0.3776 | 0.5175 | −0.8945 | −0.4705 | −0.2055 | 0.7125 | 0.1015 | 0.1275 | 0.6565 | 1.4965 | 1.0295 | 1.2295 | 0.4765 |
| STX3A | 691051 \|\| [symbol] | 0.0525 | 0.1925 | −0.8705 | −0.8485 | 0.2805 | 0.1295 | −0.6845 | −0.1345 | −0.2605 | 0.5775 | 0.2385 | 0.2185 | 0.7565 |
| STYX | 885620 \|\| [symbol] | −0.7345 | 0.3005 | −0.1295 | −0.2785 | −0.2095 | 0.4025 | −0.3385 | −0.7695 | −0.6205 | 0.6185 | −0.2535 | −0.4315 | 0.0645 |
| SUGT1 | 693218 \|\| [symbol] | −0.834 | −0.202 | −0.301 | −0.473 | 0.471 | 0.199 | 0.443 | 0.252 | −0.367 | −0.653 | 0.058 | −0.261 | 2.808 |
| SUMF1 | 695843 \|\| [symbol] | −0.2575 | 0.4785 | −0.4565 | −0.1595 | 0.3815 | 0.3795 | 0.6065 | −0.1185 | −0.2975 | 0.7195 | 0.0575 | 0.2995 | 0.3725 |
| SUMO2 | 689403 \|\| [symbol] | −0.1635 | 2.1985 | 0.2185 | −0.2345 | 0.6195 | 0.2045 | 0.1385 | 0.2085 | −0.0025 | 0.5725 | 1.0845 | 1.5235 | 0.1965 |
| SUV39H2 | 581342 \|\| [symbol] | −0.8575 | −1.8485 | −0.8515 | −0.1035 | −3.1915 | −0.4165 | −0.3245 | −1.2055 | −0.0175 | 1.6735 | 0.4695 | 0.1145 | −0.1895 |
| SYAP1 | 881813 \|\| [symbol] | 0.1525 | 0.5155 | 1.1875 | 0.7005 | −1.0335 | −0.6705 | 0.1045 | 0.4455 | 2.1945 | 2.1335 | 1.2555 | 1.5805 | −0.2475 |
| SYCP2 | 889491 \|\| [symbol] | 0.466 | 0.589 | −1.023 | 0.037 | 0.334 | −0.3 | 1.029 | 0.957 | 0.647 | −1.092 | 0.058 | −0.403 | −1.681 |
| SYNGR1 | 886385 \|\| [symbol] | 0.2375 | −0.7716 | −0.2525 | −0.5125 | 0.1255 | −0.0345 | 0.1255 | −0.2355 | 0.1265 | 0.0175 | −0.2615 | −0.4305 | −0.3385 |
| SYTL2 | 892920 \|\| [symbol] | −0.0855 | −0.3816 | −0.2645 | −0.9645 | 0.0965 | 0.0105 | 1.0635 | −0.4145 | −0.0005 | −0.4675 | −0.1035 | 0.1895 | −0.2395 |
| TAF18 | 681153 \|\| [symbol] | −0.3425 | 0.1795 | −0.7165 | −1.0065 | −0.9575 | 0.2985 | 1.2685 | −0.4795 | 0.2255 | −1.1465 | −1.1395 | −1.0805 | −1.2125 |
| TAF8 | 687144 \|\| [symbol] | −0.048 | 0.034 | 0.54 | −0.158 | 1.391 | 1.117 | −0.442 | 0.63 | 1.01 | 1.265 | 1.194 | 1.093 | 0.636 |
| TARBP1 | 897518 \|\| [symbol] | −0.436 | 0.185 | 1.615 | 0.005 | −0.874 | −0.194 | −0.895 | 1.322 | −1.483 | 0.869 | 0.989 | 1.325 | 0.242 |
| TARS | 888090 \|\| [symbol] | −0.2135 | 0.2995 | −0.3475 | −0.2595 | −0.5095 | −0.1255 | 0.3695 | 0.2705 | 0.9525 | −0.0985 | −1.0115 | −1.0865 | −0.2985 |
| TATDN1 | 892820 \|\| [symbol] | 0.1365 | 0.2255 | 0.0755 | −0.3405 | 0.3985 | 0.3235 | 0.0205 | 0.1765 | −1.0155 | 0.6335 | −0.0775 | −0.3365 | −1.0075 |
| TBC1D12 | 889394 \|\| [symbol] | −0.6105 | −0.1095 | 0.6865 | 0.3115 | −1.8245 | −0.2475 | −0.0895 | −0.5635 | 3.3995 | 1.3075 | 0.0605 | −0.1145 | −1.3515 |
| TBC1D14 | 683264 \|\| [symbol] | −0.181 | 0.172 | 0.386 | −0.056 | −0.921 | −0.183 | −0.682 | −0.185 | 0.108 | 1.034 | −0.094 | −0.004 | 0.149 |
| TBC1D3 | 884780 \|\| [symbol] | −0.5225 | 1.4885 | 1.3875 | 2.3275 | −2.1755 | 1.8015 | −1.1105 | 1.5195 | 0.5195 | 1.0045 | −1.1045 | −1.8945 | −1.8015 |
| TBCA | 689449 \|\| [symbol] | −0.892 | −1.082 | 0.235 | −0.137 | −0.895 | −1.29 | −0.806 | 0.386 | 1.255 | −1.837 | −0.907 | 0.128 | −1.612 |
| TBX1 | 882010 \|\| [symbol] | 2.6305 | 1.7445 | 2.3055 | 3.8835 | 4.3915 | 1.8925 | 0.2415 | 3.5095 | 2.6395 | 1.2835 | 0.6945 | 1.5905 | 3.7395 |
| TBX3 | 887903 \|\| [symbol] | −0.071 | −0.838 | −0.146 | −0.245 | −0.38 | −0.625 | −0.579 | −0.335 | −0.232 | −0.752 | −0.02 | 0.028 | −0.739 |
| TCAP | 680995 \|\| [symbol] | −0.121 | −0.757 | −0.379 | −0.465 | −1.01 | −1.324 | −0.725 | −0.022 | −0.028 | −2.491 | 0.257 | 1.272 | −1.166 |
| TCEAL1 | 688759 \|\| [symbol] | −0.2185 | 0.0035 | −0.5845 | −0.0785 | −0.5935 | −0.4405 | −0.2555 | −0.0035 | 0.1015 | −0.6675 | −0.0295 | 0.8485 | −0.8135 |
| TCEAL3 | 691252 \|\| [symbol] | 0.608 | 1.041 | −1.480 | −0.512 | 0.148 | 0.784 | 0.451 | 0.878 | 0.641 | −0.835 | 1.458 | 1.104 | 1.886 |
| TCEAL4 | 687353 \|\| [symbol] | 0.005 | 0.263 | 0.107 | 0.487 | 1.547 | 1.945 | 1.411 | 0.488 | 0.158 | 0.197 | 0.325 | −0.222 | 0.342 |
| TCF15 | 885545 \|\| [symbol] | −0.555 | 0.041 | −0.086 | −0.657 | 0.283 | −0.154 | −0.102 | 0.233 | 0.348 | −0.922 | −0.277 | −0.315 | −0.349 |
| TCF3 | 689758 \|\| [symbol] | −0.26 | −0.564 | 0.674 | −0.704 | −0.32 | −0.486 | 0.354 | −0.146 | 0.389 | −0.829 | −0.058 | −0.132 | −0.43 |
| TCF7L2 | 688358 \|\| [symbol] | −0.2585 | −0.2575 | 0.5095 | 0.2635 | 0.5135 | −0.0365 | 0.3095 | −0.1235 | −0.2435 | −0.3565 | 0.2895 | −0.1915 | 0.3815 |
| TDRD7 | 695781 \|\| [symbol] | 1.8725 | 0.0235 | 1.2305 | 1.0235 | 1.2645 | 1.6625 | 1.0595 | 0.2565 | 1.0195 | 1.4565 | 0.7765 | 0.1745 | 2.5655 |
| TESK2 | 585924 \|\| [symbol] | −0.064 | 0.413 | −2.102 | −1.855 | −1.906 | −2.158 | −1.313 | −2.051 | −0.189 | 1.253 | −1.047 | −2.195 | −1.966 |
| TFAP2B | 886514 \|\| [symbol] | 0.085 | 0.703 | −0.539 | −0.023 | 0.518 | 0.186 | 1.725 | 0.183 | 0.887 | 0.881 | 0.095 | −0.639 | 0.527 |
| TFCP2L1 | 885915 \|\| [symbol] | −0.5755 | −0.9215 | −0.2785 | −0.7435 | −0.7155 | −0.8425 | 0.0075 | −0.0905 | 2.7855 | −1.1645 | −0.1355 | 0.1105 | −0.2875 |
| TFCP2L2 | 697960 \|\| [symbol] | −0.5895 | −3.6035 | 2.2445 | 0.3505 | −1.9415 | 0.3315 | 2.6425 | 0.9185 | 3.1585 | −2.8305 | −0.3925 | 0.9725 | −1.1685 |
| TFF1 | 688887 \|\| [symbol] | −0.8535 | 0.9425 | 1.1765 | −0.9465 | 0.9585 | 0.2215 | −0.1465 | 0.9855 | 0.1435 | 1.2285 | 1.1085 | 0.2925 | 0.5325 |
| TFF3 | 695605 \|\| [symbol] | 0.639 | −0.254 | −1.05 | −0.974 | −0.952 | −1.623 | −0.663 | −0.644 | −0.738 | −0.461 | −0.695 | −0.788 | −1.111 |
| TFRC | 693432 \|\| [symbol] | −0.1875 | −0.2145 | −0.4835 | −0.1865 | 0.5805 | 0.3115 | −0.7885 | 0.0865 | −0.1955 | −0.1105 | −0.5525 | −0.7855 | −0.5995 |
| TGFBR3 | 690641 \|\| [symbol] | −0.08 | −0.16 | −0.732 | −0.012 | 0.298 | −0.04 | 1.176 | −0.439 | 0.402 | −0.607 | −0.41 | −0.69 | 0.378 |
| THY28 | 687185 \|\| [symbol] | −0.3165 | −0.3755 | −0.5795 | 0.0285 | −1.8235 | −1.0875 | −1.6845 | −0.7755 | −0.1565 | −0.7245 | 0.0735 | −0.5885 | 0.4065 |
| TIF1 | 682191 \|\| [symbol] | −0.445 | −0.415 | −0.02 | −0.485 | −0.233 | −0.134 | 0.179 | −0.554 | 0.013 | 0.120 | −0.814 | −0.873 | −0.37 |
| TIGA1 | 689331 \|\| [symbol] | −0.44 | −0.417 | −0.074 | 0.349 | −0.21 | 0.568 | 0.264 | −0.027 | −0.216 | 0.72 | 0.392 | 0.55 | 0.882 |
| TIM14 | 681121 \|\| [symbol] | −0.0105 | 0.0925 | −0.1685 | 0.3485 | −0.3105 | 0.1255 | −0.8455 | 0.1645 | 0.2235 | 0.3325 | 0.0575 | −0.3295 | 0.4445 |
| TIMELES [symbol] | 089106 \|\| [symbol] | −0.4875 | 0.3205 | −0.1675 | −0.3775 | −0.4815 | −0.4265 | −1.1355 | 0.0865 | 0.0235 | 0.5315 | 0.1495 | 0.3025 | −0.2555 |
| TIMM9 | 684722 \|\| [symbol] | 0.113 | −0.29 | −1.008 | −0.141 | −0.916 | 3.892 | −0.075 | −0.85 | −0.361 | −1.159 | −1.452 | −1.004 | −1.474 |
| TINP1 | 688051 \|\| [symbol] | −0.7115 | −0.5025 | 0.0595 | 0.5115 | 0.0035 | 0.4215 | 1.5815 | −0.0265 | −0.5085 | 0.8095 | 0.1555 | 0.1625 | 1.1745 |
| [symbol] IP1208 | 688908 \|\| [symbol] | −0.029 | −0.41 | −0.483 | −0.988 | −1.571 | 0.321 | −0.65 | −0.54 | −0.307 | −1.515 | −0.721 | 0.097 | −0.358 |
| TK1 | 688069 \|\| [symbol] | 0.892 | −0.589 | −0.288 | −0.198 | −0.657 | −0.83 | −0.003 | −0.413 | −0.294 | −0.753 | −0.587 | −0.041 | −1.229 |
| TLE2 | 090798 \|\| [symbol] | 0.08 | 0.019 | −1.025 | −0.837 | 0.384 | 2.124 | −1.169 | 0.004 | 0.623 | 0.147 | −1.212 | −0.624 | −1.685 |
| TLR5 | 682772 \|\| [symbol] | −1.469 | 0.968 | −0.172 | −0.726 | −0.105 | 0.833 | −0.899 | 0.057 | 0.886 | 0.024 | 0.398 | 0.047 | 0.272 |
| TM4SF12 | 689572 \|\| [symbol] | −0.483 | −0.851 | −0.081 | −1.014 | 0.353 | 0.045 | −0.441 | −0.221 | −0.055 | 0.301 | 0.05 | 0.105 | −0.747 |
| TM4SF13 | 696352 \|\| [symbol] | −1.01 | −0.998 | −1.042 | 0.082 | −0.758 | −0.059 | 0.452 | −0.801 | 0.048 | 0.57 | −0.961 | −0.19 | 0.124 |
| TMEM14A | 883783 \|\| [symbol] | −0.0355 | −0.0655 | −0.4215 | 0.6425 | 0.0765 | −0.0055 | 0.0785 | 0.2045 | 0.7595 | 0.0385 | 0.0995 | 0.2385 | −0.3155 |
| TMEM25 | 697985 \|\| [symbol] | −0.2025 | −0.7865 | −0.4795 | −0.1515 | −0.1825 | −0.3425 | −0.1685 | −0.5635 | −0.4205 | −0.8765 | 0.1375 | −0.6075 | 0.7415 |
| TMEM4 | 695931 \|\| [symbol] | 0.494 | 0.551 | −0.223 | 1.823 | 0.752 | 1.407 | 2.278 | 1.14 | 1.064 | 0.407 | 0.809 | 0.711 | 0.479 |
| TMPRSS5 | 889588 \|\| [symbol] | −0.011 | 1.6 | 0.1 | 0.68 | 0.045 | 1.107 | 1.827 | 1.038 | −0.308 | 0.803 | 1.663 | 0.26 | −0.907 |
| TMSB10 | 695386 \|\| [symbol] | −0.989 | −0.485 | −0.68 | −0.044 | 1.275 | −0.15 | 0.742 | −1.612 | −0.856 | 0.188 | 0.099 | −0.022 | −0.211 |
| TMSNB | 681721 \|\| [symbol] | −0.3705 | 0.5735 | −0.1605 | −0.5485 | 0.5805 | −0.7715 | −0.2915 | 0.9305 | 0.1405 | −0.6035 | 0.2545 | 0.2235 | −0.2335 |
| TNFRSF1 [symbol] | 681592 \|\| [symbol] | 0.518 | 0.475 | 0.573 | −0.582 | 1.021 | −0.485 | −0.459 | 0.777 | 0.014 | −0.497 | 0.235 | −0.094 | −0.689 |
| TNFRSF2 [symbol] | 689952 \|\| [symbol] | −0.6075 | 1.1245 | −0.9545 | −0.8305 | 0.3145 | 0.8015 | 1.4465 | 1.5355 | 1.9205 | 0.5935 | −0.3105 | −0.8835 | −0.0085 |
| TNFRSF2 [symbol] | 692465 \|\| [symbol] | 4.5275 | 0.8835 | −0.3135 | 3.8485 | −0.4785 | −0.7805 | 3.1885 | −0.5515 | 0.2865 | 3.2465 | 1.8805 | 2.7455 | 1.9335 |
| TNFSF4 | 094020 \|\| [symbol] | 0.4285 | 0.3205 | 0.7155 | 0.6835 | 1.6855 | 0.2815 | 0.7485 | 0.2925 | 0.5275 | −0.0545 | 0.1515 | 0.7325 | −0.0015 |
| TNFSF7 | 684958 \|\| [symbol] | 0.5085 | −0.8915 | −0.6475 | 0.3465 | 0.3435 | 1.1235 | 1.3155 | −0.5725 | −1.0925 | 0.0685 | 0.5055 | −0.0395 | 0.2215 |
| TNIK | 695447 \|\| [symbol] | 0.7165 | 0.6615 | −0.2495 | −1.1195 | −0.8505 | −0.4455 | −0.1065 | −0.4125 | −0.6635 | −0.6445 | −0.1255 | −1.1935 | −0.1685 |
| TNNC2 | 687349 \|\| [symbol] | −1.018 | −0.009 | −0.05 | 0.182 | −0.177 | −0.322 | 2.336 | 0.685 | −0.666 | −0.428 | −0.478 | −0.323 | 0.332 |
| TNNI2 | 887679 \|\| [symbol] | −0.861 | −0.485 | −0.503 | 1.17 | −0.7 | −0.518 | 0.087 | −1.32 | −0.147 | −0.006 | −0.193 | 0.984 | 0.875 |
| TNNI3 | 695629 \|\| [symbol] | −0.5055 | −0.9235 | −0.6855 | −0.5875 | −0.3565 | −0.5845 | 0.4575 | −0.1905 | −0.4485 | 0.0915 | −0.2085 | −1.0235 | 0.7245 |
| TOB1 | 683402 \|\| [symbol] | 0.2765 | 0.1565 | −0.7785 | 0.3385 | −0.6545 | −0.0315 | −0.2025 | −0.0575 | −0.4625 | −0.0375 | 0.0455 | 0.1405 | −0.6815 |
| TOM1L1 | 685320 \|\| [symbol] | −0.3295 | 1.1905 | −1.3165 | −0.3965 | −0.3285 | 0.1085 | −0.7425 | −0.1165 | −0.4535 | 0.6585 | 1.2455 | 0.8555 | 0.9895 |
| TOMM7 | 683060 \|\| [symbol] | −1.0405 | −0.1855 | 1.2045 | −0.8175 | −0.8555 | 0.0655 | 0.1835 | −0.3145 | −0.0695 | −0.8135 | −0.3995 | −0.8585 | −0.1475 |
| TOP1MT | 693021 \|\| [symbol] | −0.381 | 0.498 | −0.148 | −0.64 | −0.24 | 1.201 | 1.1 | −0.359 | −0.421 | −0.125 | −0.025 | −0.442 | −0.33 |
| TOP2A | 690278 \|\| [symbol] | −0.3165 | 0.0675 | −0.2815 | −0.7685 | −0.5985 | 0.1485 | 0.0615 | −0.0575 | −0.1015 | 0.7865 | −0.2505 | −0.4795 | −0.3205 |
| TOP2B | 687326 \|\| [symbol] | −0.991 | −1.654 | 0.617 | −0.485 | −0.555 | 0.771 | 0.492 | −0.325 | −0.154 | 0.067 | 0.001 | −0.368 | 0.452 |
| TOPBP1 | 897492 \|\| [symbol] | 0.8365 | −0.9665 | −1.0925 | 1.0825 | 1.3945 | 0.5965 | 3.0405 | −1.0845 | −1.0555 | −0.2625 | −0.1075 | 0.3885 | 0.8355 |
| TOPK | 884492 \|\| [symbol] | −0.4015 | 1.1375 | −0.3415 | −0.6055 | −0.1925 | 1.2345 | 1.8185 | −0.1175 | 0.4685 | −0.8125 | 0.2505 | −0.2355 | −0.6235 |
| TP53AP1 | 888947 \|\| [symbol] | −0.2125 | −0.3295 | −0.0785 | 0.4155 | 0.7415 | 0.3215 | 0.4135 | −0.2285 | −0.1175 | −0.8275 | 0.0725 | 0.2445 | 0.5965 |
| TP53BP2 | 681831 \|\| [symbol] | 0.2295 | 0.2115 | −0.6405 | −0.5385 | −0.2695 | −0.2085 | −0.7105 | −1.0545 | −0.7685 | 2.0675 | 1.4115 | 1.8445 | −3.4235 |
| TP53INP1 | 686814 \|\| [symbol] | −0.3115 | 0.0515 | 0.4105 | 0.2245 | 0.4575 | 0.0105 | 0.1525 | −0.0155 | 0.0615 | 2.0305 | 0.8635 | 0.1355 | 0.7885 |
| TPD52L1 | 884176 \|\| [symbol] | 0.462 | 0.159 | −1.695 | −0.431 | −1.485 | 0.332 | 0.238 | −0.467 | −0.501 | 0.541 | 0.388 | −0.13 | −0.678 |
| TPRT | 695363 \|\| [symbol] | 0.4265 | −0.7915 | 1.4676 | 2.7685 | 2.8385 | 1.9715 | 0.4605 | 0.0655 | 0.1975 | −0.2305 | 0.5635 | 0.6285 | 2.6435 |
| TRA@ | 696810 \|\| [symbol] | −0.015 | −0.464 | −0.213 | 0.089 | −0.412 | 0.042 | −0.242 | −0.101 | 0.04 | −0.05 | 0.063 | 0.035 | 0.431 |
| TRAF4 | 695150 \|\| [symbol] | −0.454 | −0.155 | 0.581 | 1.105 | 0.001 | −0.078 | −0.179 | 0.039 | −0.081 | −0.073 | −0.83 | 0.892 | −0.556 |
| TRAPPC4 | 682554 \|\| [symbol] | 0.232 | −0.182 | 0.192 | −0.044 | −0.662 | −0.625 | −0.801 | −0.493 | −0.227 | −0.458 | −0.447 | −0.285 | −0.344 |
| TRH | 688633 \|\| [symbol] | −0.3705 | 0.0485 | −0.0495 | −0.3035 | −1.1565 | −0.2195 | −0.6845 | −0.4535 | −1.2765 | 0.3455 | 0.0635 | −1.1095 | −1.3515 |
| TRIM17 | 884736 \|\| [symbol] | 0.1455 | 0.9895 | 0.3835 | −1.1755 | −0.9865 | −0.8045 | 0.0785 | −0.2205 | 0.0285 | 0.5235 | 0.2255 | 0.4775 | −0.7605 |
| TRIM2 | 882053 \|\| [symbol] | −0.3595 | 1.1495 | −1.0695 | −0.3445 | −0.7985 | −0.3215 | −0.3835 | −0.5245 | −0.6375 | 0.5315 | 0.5075 | 0.1875 | 2.8275 |
| TRIM6 | 882620 \|\| [symbol] | −1.503 | −1.122 | 0.558 | −1.462 | −1.762 | −0.524 | −3.944 | −0.903 | −0.387 | 0.193 | −2.704 | −0.385 | −1.638 |
| TRIP3 | 696359 \|\| [symbol] | −0.2905 | −0.3555 | −0.6245 | −0.5255 | 0.1805 | 0.0335 | −0.1345 | −0.4315 | 0.1625 | 0.1365 | −0.6735 | −0.4395 | −0.4585 |
| TRPS1 | 883107 \|\| [symbol] | 0.1575 | −0.4235 | −0.2875 | −1.0335 | −0.2475 | 0.5165 | −0.8945 | −0.1445 | 0.0475 | −0.1845 | −0.6195 | −1.3595 | −0.5205 |
| TSG101 | 885755 \|\| [symbol] | 0.0755 | −0.0815 | −0.6745 | 0.7715 | 0.1215 | 0.0205 | −0.3985 | −0.5475 | 0.3485 | −0.8925 | −0.1565 | 0.5805 | 0.7105 |
| TTC10 | 687943 \|\| [symbol] | 0.0935 | −0.0815 | −0.3825 | −1.1545 | −0.2705 | 0.3145 | 0.8615 | 0.0445 | 0.4025 | 1.0685 | 0.9285 | 0.2115 | 0.5705 |
| TTC12 | 882598 \|\| [symbol] | 0.481 | 0.595 | 0.174 | −0.135 | 0.096 | −0.485 | 0.096 | −0.176 | −0.157 | −0.3 | 0.458 | −0.1 | 0.01 |
| TTK | 691976 \|\| [symbol] | 0.4525 | −0.8155 | 0.5725 | 2.2125 | −0.1395 | −0.4475 | 0.9995 | −0.9255 | −0.7795 | −0.3455 | 0.1835 | 0.7445 | 0.8205 |
| TTLL4 | 685109 \|\| [symbol] | 1.455 | 1.21 | 2.045 | 0.672 | 0.564 | −0.197 | −0.165 | 0.888 | 0.042 | −0.78 | 0.955 | 0.187 | 2.726 |
| TTYH1 | 686941 \|\| [symbol] | 0.5625 | −0.5155 | −0.9085 | 0.6305 | −0.3795 | 0.2395 | −0.2245 | −0.1745 | 1.4795 | −0.5585 | −0.1635 | −0.6585 | 0.6725 |
| TUBB3 | 885420 \|\| [symbol] | 0.6095 | −0.2575 | 0.4995 | 1.0185 | 0.4915 | 0.6605 | 0.3755 | −0.1605 | 0.6845 | −0.5445 | 0.1365 | −0.1235 | −0.2715 |
| TULP3 | 884031 \|\| [symbol] | −0.335 | −0.805 | −0.321 | −1.464 | −1.205 | −0.005 | −0.085 | −0.055 | 0.095 | 0.125 | −0.005 | −0.484 | −0.035 |
| TXN | 687357 \|\| [symbol] | −0.198 | −0.236 | −0.353 | 0.288 | 0.08 | 0.468 | −0.109 | −0.014 | −0.304 | −0.187 | −0.642 | −0.733 | 0.183 |
| TYMS | 763590 \|\| [symbol] | 0.0785 | 1.1495 | 0.0005 | −1.3135 | 1.1995 | −0.3665 | 0.9455 | −0.1185 | −0.8835 | −0.4465 | 0.6995 | −0.5955 | −0.0005 |
| U5-116KD | 896228 \|\| [symbol] | −0.3285 | −0.7625 | −0.4585 | −0.0155 | 0.2315 | 0.2855 | 0.2305 | −0.1955 | 0.2655 | 0.5035 | 0.0315 | 0.3635 | 0.0585 |
| UBE2E3 | 882401 \|\| [symbol] | −0.113 | 0.451 | −0.655 | 0.142 | 1.26 | 0.322 | 0.433 | −0.393 | −0.023 | 0.392 | 0.016 | 0.315 | 0.647 |
| UBL5 | 888745 \|\| [symbol] | 1.0255 | 0.5855 | 1.7285 | 2.6805 | 0.4325 | 0.5395 | 0.0365 | 0.8045 | 0.7015 | −1.0595 | −0.2685 | 0.1975 | −1.1905 |
| UBN1 | 887671 \|\| [symbol] | 1.1 | 0.185 | −0.059 | 0.067 | 1.548 | −0.11 | −0.404 | −0.326 | −0.01 | 1.237 | 1.317 | 1.019 | 0.801 |
| UCHL1 | 888791 \|\| [symbol] | 1.094 | −0.71 | 1.265 | 0.522 | −1.069 | −0.242 | −0.873 | −0.899 | −0.535 | −1.171 | −1.149 | −1.368 | −0.329 |
| UCHL3 | 695206 \|\| [symbol] | 0.483 | 0.683 | 0.068 | 0.774 | −0.172 | 0.432 | 0.993 | 0.258 | 0.466 | 1.084 | 1.643 | 0.882 | 0.992 |
| UGCG | 682938 \|\| [symbol] | 4.5175 | −0.3235 | −0.0465 | 5.5375 | 1.0295 | 0.3475 | 6.0285 | 0.8215 | −0.0445 | 5.2485 | 1.9165 | 1.4095 | 5.7985 |
| UGCGL1 | 688132 \|\| [symbol] | −0.3205 | −0.3035 | 0.2375 | −0.4715 | 0.3055 | −0.0735 | 0.2655 | −0.4345 | −0.5375 | 0.1835 | −0.1335 | −0.1045 | 0.7655 |
| UGT2B7 | 889581 \|\| [symbol] | 0.0505 | −0.3075 | 0.5035 | 0.6575 | −0.0035 | 0.9225 | 1.0455 | −0.8725 | −0.2445 | 0.3225 | 0.3675 | 0.9485 | 0.2085 |
| UNC13D | 893880 \|\| [symbol] | −0.486 | 0.063 | −0.084 | −0.59 | −0.523 | −0.811 | −0.07 | 0.343 | −0.789 | 0.6 | −0.192 | −0.121 | −1.217 |
| VAMP8 | 685078 \|\| [symbol] | −0.104 | 0.537 | −0.21 | −0.897 | −0.341 | −0.124 | 0.359 | 0.027 | −0.073 | 0.173 | 0.019 | 0.294 | −0.545 |
| VAPA | 586453 \|\| [symbol] | −0.5735 | 0.4505 | −0.3095 | 1.0855 | 0.3285 | −1.0805 | 0.2825 | −1.0685 | −2.7185 | −1.8625 | −0.1265 | −0.2775 | −0.5925 |
| VAV1 | 593880 \|\| [symbol] | 0.416 | −0.683 | 0.114 | 1.214 | −0.216 | −0.052 | 3.352 | 1.302 | −0.88 | 0.491 | −0.824 | −0.926 | −0.521 |
| VAV3 | 695745 \|\| [symbol] | −0.265 | −0.443 | 0.329 | −0.142 | 0.229 | −0.091 | −0.098 | 0.291 | 0.004 | 0.318 | 0.942 | 1.032 | −0.117 |
| VCX3 | 888317 \|\| [symbol] | 0.469 | 0.008 | −0.02 | 0.234 | −0.193 | −0.089 | −0.428 | 1.761 | 0.114 | 0.162 | −0.332 | −0.049 | −0.394 |
| VDAC2 | 681387 \|\| [symbol] | 1.0085 | 0.6055 | 0.2435 | −0.1805 | −0.2915 | 0.8985 | −0.0915 | −0.4418 | −0.2015 | 0.1985 | −0.2315 | −1.1855 | 0.6985 |
| VEST1 | 685356 \|\| [symbol] | −0.309 | −0.344 | 0.508 | 1.11 | −0.251 | 0 | −0.187 | −0.02 | 0.02 | 0.015 | 0.516 | 0.575 | 0.837 |
| VGLL1 | 703309 \|\| [symbol] | 1.1175 | 0.1685 | −0.3585 | 0.3005 | −1.0615 | 0.1035 | −0.7915 | −0.6585 | 0.2785 | −0.1075 | −0.8085 | −0.3345 | −0.9715 |
| VMD2L2 | 896188 \|\| [symbol] | 0.234 | 0.499 | 0.969 | 0.866 | 0.112 | 0.155 | 0.481 | −0.113 | −0.659 | 0.416 | 0.081 | 0.356 | 0.595 |
| VSX1 | 692722 \|\| [symbol] | −0.089 | −0.016 | 0.951 | −0.48 | 0.125 | 1.178 | 0.962 | 0.714 | 0.47 | −0.113 | −0.169 | −0.497 | 0.009 |
| WARP | 683039 \|\| [symbol] | −0.336 | 0.321 | −0.707 | −0.224 | −1.124 | −0.298 | −0.001 | −0.329 | −0.637 | −0.678 | −0.268 | −0.45 | 0.035 |
| WDR26 | 681481 \|\| [symbol] | −0.3385 | −0.6025 | −1.4515 | −0.8425 | 0.0676 | 0.2965 | −0.2165 | −0.4195 | 0.5415 | −0.9115 | 0.4235 | 0.8415 | −1.0615 |
| WDR6 | 693627 \|\| [symbol] | −0.826 | 0.288 | −0.796 | −0.446 | −0.793 | −0.117 | −0.759 | −0.812 | 0.229 | −1.316 | 0.019 | −0.108 | −0.917 |
| WHSC1L1 | 895912 \|\| [symbol] | −0.009 | 0.679 | −0.01 | 0.653 | 0.161 | −0.489 | −0.259 | 0.051 | −0.418 | −0.489 | 0.171 | 0.205 | −0.199 |
| WNT3 | 888424 \|\| [symbol] | 0.7475 | 0.0095 | 0.3925 | 0.8585 | 0.5375 | 1.0415 | 0.8395 | 0.8185 | 0.3845 | 1.1825 | 0.5765 | 0.3795 | 0.4685 |
| WNT6 | 763326 \|\| [symbol] | −0.3525 | 0.4705 | 0.4195 | −1.0645 | −0.6955 | −0.5445 | −0.1975 | −0.7035 | −1.0865 | −0.2975 | 0.5015 | −0.0285 | 0.4115 |
| WSB2 | 893380 \|\| [symbol] | 0.7385 | 0.4465 | 0.2555 | 0.4875 | 0.3585 | 0.2465 | −0.1255 | 0.5845 | −0.4445 | 0.0325 | −0.1955 | 0.3805 | −0.7775 |
| WWP1 | 686545 \|\| [symbol] | −0.5185 | −0.6005 | 0.3165 | 0.4945 | −1.9045 | −0.7945 | 0.3015 | −0.2845 | 0.8025 | −0.7555 | −0.6445 | 0.1865 | 1.1335 |
| WWP2 | 691487 \|\| [symbol] | −1.03 | −0.482 | 2.811 | 0.796 | −0.567 | 1.129 | 0.606 | −0.288 | 0.258 | −0.976 | 1.01 | 1.293 | 1.531 |
| XBP1 | 989595 \|\| [symbol] | −0.256 | 0.345 | 0.131 | −0.125 | −0.992 | 0.675 | 0.874 | −0.19 | −0.476 | 1.694 | 1.215 | 0.57 | 1.457 |
| XYLT2 | 892339 \|\| [symbol] | −0.5235 | 0.4975 | −0.1195 | −0.2065 | 0.2895 | −0.0045 | −0.3415 | 0.6765 | 0.6755 | 0.3015 | 0.6305 | 0.6955 | −0.1345 |
| YBX2 | 890318 \|\| [symbol] | −0.088 | 0.121 | 0.700 | −0.344 | −0.475 | −0.724 | −0.592 | 0.291 | −0.017 | −0.078 | 0.148 | 0.159 | −0.548 |
| YIF1 | 687006 \|\| [symbol] | 0.5585 | 1.0905 | −0.0139 | 0.4855 | −0.7425 | −0.1785 | 0.0116 | 0.2095 | 0.9275 | 0.2295 | −0.9335 | −0.6505 | −0.6635 |
| ZA20D3 | 694225 \|\| [symbol] | −0.4635 | −0.3535 | −0.4395 | 0.0485 | −0.4625 | −0.3815 | −0.9215 | 0.1285 | 0.1815 | 0.6795 | 0.0785 | 0.4105 | −0.8735 |
| ZAP128 | 697114 \|\| [symbol] | 0.1465 | 0.1405 | −0.3885 | −0.1845 | −0.8555 | −0.0185 | −0.5275 | −0.1665 | −0.5475 | −0.1755 | −0.7105 | −0.3775 | −0.8705 |
| ZBED3 | 694304 \|\| [symbol] | 0.214 | −0.128 | −0.708 | 0.498 | 0.827 | 0.459 | 0.751 | 0.144 | 0.766 | −0.331 | −0.148 | 0.065 | 1.850 |
| ZBTB4 | 692879 \|\| [symbol] | 0.747 | 0.184 | 0.182 | 0.958 | 0.477 | 0.713 | 1.22 | 0.509 | 0.93 | 0.56 | 0.236 | 0.567 | 0.077 |
| ZCWCC1 | 694671 \|\| [symbol] | −0.6395 | 0.4825 | −0.4335 | −0.2235 | 0.1705 | 0.5665 | −0.6245 | −0.2165 | 0.0655 | 0.0615 | −0.4925 | −1.1045 | −0.4095 |
| ZCWCC2 | 688993 \|\| [symbol] | 1.415 | −0.745 | −0.481 | 1.276 | 1.502 | −0.42 | 0.953 | −1.053 | −0.204 | 0.54 | −0.011 | 0.032 | −1.953 |
| ZDHHC13 | 692887 \|\| [symbol] | 0.5705 | 0.9865 | −1.5985 | −0.9735 | −1.0535 | 0.2045 | 0.0845 | 1.1345 | 0.2445 | −0.8725 | −0.4335 | −1.4605 | −0.8365 |
| ZDHHC14 | 690617 \|\| [symbol] | 0.0435 | 0.5075 | −0.1275 | 0.8995 | 0.3695 | −0.0145 | 0.0815 | 0.3885 | 0.6605 | 0.1895 | −0.3595 | −0.8965 | −0.0205 |
| ZFHX4 | 698157 \|\| [symbol] | −0.0035 | −0.3295 | −0.5935 | −0.0015 | 0.0735 | 0.4275 | 0.2125 | 0.7665 | 0.2875 | −0.1225 | −0.4405 | −0.1305 | 0.3115 |
| ZFYVE21 | 696639 \|\| [symbol] | −0.016 | −0.236 | 1.187 | 0.054 | −0.213 | −0.225 | −0.898 | −0.873 | −0.16 | −0.007 | −0.098 | 0.854 | 0.265 |
| ZNF133 | 689721 \|\| [symbol] | −0.117 | −0.91 | −0.009 | 0.108 | 0.759 | 0.144 | −0.853 | 0.069 | 0.695 | −0.859 | −0.477 | 0.326 | −0.309 |
| ZNF165 | 688726 \|\| [symbol] | −0.3125 | −0.3375 | −1.1075 | −0.1485 | −0.3775 | 0.3885 | 0.3525 | −0.5485 | 0.0165 | −0.7275 | −0.2215 | −0.3195 | 0.3285 |
| ZNF24 | 683142 \|\| [symbol] | −0.419 | 0.238 | −0.283 | −0.879 | −0.325 | −0.214 | −0.308 | 0.192 | −0.114 | −0.443 | 0.027 | 0.076 | −0.672 |
| ZNF318 | 682803 \|\| [symbol] | −0.0015 | −0.1585 | 0.3105 | 0.2325 | −0.1085 | 0.0145 | 0.6915 | 0.1145 | 0.0985 | 0.2335 | −0.2105 | −0.3025 | 0.7355 |
| ZNF354 | 690750 \|\| [symbol] | −0.7655 | 0.9575 | 0.4525 | −1.3455 | 0.2955 | −1.0205 | −1.3275 | −0.2775 | 0.6885 | −1.6915 | 0.1265 | −1.6985 | −1.6595 |
| ZNF432 | 896694 \|\| [symbol] | −0.4275 | 0.1545 | −0.0165 | 0.6615 | 0.9525 | 0.5365 | 0.9715 | 0.9705 | 0.1205 | 0.2115 | 0.8185 | 0.9995 | 0.9745 |
| ZNF482 | 882655 \|\| [symbol] | −1.456 | −0.396 | 0.618 | −1.092 | 1.211 | 0.119 | 0.591 | −0.684 | −0.761 | −0.55 | −0.43 | −0.689 | −0.198 |
| ZNF505 | 697563 \|\| [symbol] | 0.36 | −0.228 | 0.876 | 0.954 | −0.453 | 0.594 | 0.936 | 0.265 | −0.205 | −0.801 | −0.139 | −0.012 | 1.402 |
| ZNF552 | 681805 \|\| [symbol] | 0.1855 | −0.0485 | 0.0005 | −0.4765 | −0.0235 | 0.2685 | −0.0065 | −0.2525 | 0.1495 | 0.0075 | −0.7665 | −1.7185 | 0.3216 |
| ZNF577 | 696450 \|\| [symbol] | 0.2715 | 0.5475 | 0.2605 | −0.2005 | 0.3545 | 0.1785 | 0.8705 | 0.2755 | 0.7535 | 0.8255 | 0.1185 | −0.0076 | 0.2695 |
| ZNF587 | 695267 \|\| [symbol] | −0.3165 | −0.3925 | −1.1345 | −0.1845 | −2.1765 | −0.0595 | 1.0825 | −0.1965 | −0.2425 | −0.3275 | −1.1735 | −1.3495 | −0.5925 |
| ZNF598 | 689656 \|\| [symbol] | −0.2445 | −0.0225 | 0.0615 | −0.0135 | 0.6925 | 0.7455 | 0.6955 | −0.1055 | −0.5475 | −0.7885 | −0.2395 | −0.3465 | 0.5845 |
| ZNF599 | 697181 \|\| [symbol] | −0.278 | 0.614 | 0.548 | 0.092 | −0.21 | −0.019 | 0.84 | −0.108 | 0.193 | −0.226 | 0.334 | 0.083 | 1.395 |
| ZNF606 | 883801 \|\| [symbol] | −0.525 | 0.535 | 0.543 | −0.805 | −0.351 | −0.251 | 0.231 | 0.081 | 0.117 | 0.064 | −0.289 | −0.538 | −1.489 |
| ZNF614 | 683556 \|\| [symbol] | −0.168 | 0.734 | −1.066 | −0.771 | −0.553 | 0.535 | −0.37 | 0.892 | −0.709 | 0.218 | 1.25 | 1.385 | 0.864 |

| ZNF639 | 690004 \|\| ZNF639 \|\| Zinc finger protein 639 \|\| Hs.22879 \|\| AF003924 \|\| \|\| 51193 \|\| \|\| \|\| NM_016331 \|\| \|\| \|\| \|\| | | | | | | | | | | | | | | |
|---|---|---|---|---|---|---|---|---|---|---|---|---|---|---|---|
| ZNF7 | 684715 \|\| ZNF7 \|\| Zinc finger protein 7 (KOX 4, clone HF.16) \|\| Hs.493218 \|\| NM_003418 \|\| \|\| 7553 \|\| | | | | | | | | | | | | | | |
| | ENSG00000147789 \|\| ENST00000333208\|\|ENST00000328072\|\|ENST00000325948\|\|ENST00000325241\|\|ENST00000278814 | | | | | | | | | | | | | | |
| | \|\| NM_003416 \|\| \|\| chr8: 146073063-148073122 \|\| 8q24.3 \|\| | | | | | | | | | | | | | | |
| | Sample | | | | | | | | | | | | | | |
| | BR01-012 [symbol] | BC00055 | BC00071 | BC00074 | BC00097 | BC00010 | UB-117 | UB-118 | BR94-108 [symbol] | UB49 | BC00070 | UB-118 | PB377 | BR97-013 [symbol] | PB418 |
| | Class | | | | | | | | | | | | | | |
| Symbol | HER2 | HER2 | HER2 | HER2 | HER2 | HER2 | HER2 | HER2 | HER2 | HER2 | BASAL | BASAL | BASAL | BASAL | BASAL |
| | 0.0915 | −1.6875 | −1.4115 | −1.5255 | −0.0815 | −1.1825 | −1.1375 | −1.0425 | 2.2065 | −0.7095 | −2.5035 | −1.8045 | −1.9585 | −1.2015 | −2.7995 |
| | −0.373 | 0.551 | −0.494 | 0.475 | −0.916 | −0.757 | 0.912 | 0.514 | −0.494 | 0.425 | −0.228 | −0.11 | −0.04 | −0.112 | −0.488 |
| ABAT | 2.312 | 1.242 | 0.663 | 1.019 | −1.327 | 0.539 | 2.232 | 0.228 | 0.812 | −1.948 | −1.067 | −2.852 | 0.034 | −2.83 | −2.675 |
| ABC1 | 0.2735 | 3.9385 | 1.4875 | 0.9995 | 0.8985 | 3.0575 | 3.4395 | 0.0085 | −0.6405 | −0.7425 | −0.7475 | −0.5085 | 0.3315 | −0.9375 | −0.8865 |
| ABCC11 | 2.2886 | −0.8205 | −1.2925 | −1.7375 | −0.0075 | 0.8325 | −0.7645 | 0.6135 | −1.1225 | −0.0895 | −0.1375 | 1.3175 | 0.4465 | −0.9195 | 0.0215 |
| ABCC3 | −0.002 | 0.447 | 0.835 | 0.014 | 0.938 | −0.077 | −0.051 | −0.367 | 0.814 | −0.01 | −0.282 | 0.127 | −0.378 | 0.016 | 0.629 |
| ACAS2L | 0.6795 | 0.8525 | −0.1335 | 0.2035 | −1.6065 | 0.5365 | −0.1325 | 0.1145 | 0.1055 | 0.0225 | 0.9775 | 1.0075 | −0.5575 | 0.7145 | −0.1395 |
| ACAT1 | −1.348 | 2.927 | 2.213 | 0.491 | −0.959 | 0.311 | −2.136 | −1.311 | −1.138 | −1.558 | −2.476 | −2.318 | −1.98 | −2.613 | −0.576 |
| ACAT2 | 1.7215 | −0.9085 | −0.8975 | −0.7845 | 0.2555 | −0.2105 | −0.8285 | 0.7875 | 0.1845 | 0.0525 | −0.0495 | 0.8115 | 0.0915 | −0.0895 | 0.6335 |
| ACOX2 | −0.9865 | −0.2235 | 0.0135 | 0.6795 | 0.3535 | −1.0925 | −1.0385 | −0.4855 | −0.9395 | −0.9275 | −1.1365 | −0.9245 | −0.2325 | −0.9085 | −0.7835 |
| ACDX3 | −0.0695 | 2.0445 | 3.0745 | −0.4995 | 0.9435 | −0.8445 | −0.0935 | −0.2295 | 0.8205 | 0.0565 | 3.0245 | 6.3295 | 0.3435 | −0.1395 | 0.3785 |
| ACSL3 | 0.793 | 0.815 | 1.342 | 0.876 | 0.317 | 1.081 | 1.55 | 0.509 | −0.009 | 0.766 | 1.409 | 1.176 | 0.591 | 0.48 | −0.149 |
| ACTA1 | 0.0085 | 0.0365 | −0.1655 | −0.3235 | 0.2965 | 0.4775 | 0.2835 | −0.1935 | 0.1935 | 0.0555 | −0.0905 | −0.1915 | 0.3885 | 0.0085 | −0.2275 |
| ACTB | −0.5925 | −0.5655 | −0.7535 | −0.5595 | 0.2525 | 2.8695 | 1.6785 | 1.9915 | −0.6305 | −0.8245 | −0.0665 | −0.0235 | 0.3105 | −1.3595 | −0.5625 |
| ADAM33 | 1.306 | 0.055 | 1.407 | 0.848 | −0.258 | 0.013 | 2.414 | −0.29 | 2.252 | −0.757 | 0.867 | 0.867 | −0.808 | 1.602 | −1.214 |
| ADAMDE [symbol] | −0.7075 | 1.0875 | −0.7585 | 0.1855 | −0.3495 | −0.4885 | 0.7475 | −0.6135 | −0.0565 | −0.1585 | −0.4505 | −0.6395 | −0.4775 | −0.1595 | −0.4865 |
| ADPN | −0.8855 | 0.8415 | 0.9215 | −0.5285 | −0.7305 | 0.4675 | −0.3485 | 1.1905 | 0.3085 | 1.7805 | −1.3205 | 1.0255 | 1.0815 | −1.0635 | −0.7495 |
| ADPRHL1 | −3.1885 | −1.3935 | 0.2395 | −0.2705 | −0.6545 | −0.5285 | 1.7955 | 1.3805 | −1.5725 | 0.4555 | −3.3185 | −3.1775 | −3.4775 | −3.5805 | −4.3805 |
| ADRA2C | 1.8975 | −0.5395 | 1.1175 | 0.8985 | −0.7265 | −0.8195 | −0.1905 | −0.3905 | 1.2435 | 1.3755 | −0.2485 | −1.1515 | 4.1065 | −0.1795 | −0.4475 |
| AGR2 | −0.8305 | −0.2505 | −0.4705 | −0.4115 | −0.5875 | −0.4275 | −0.0785 | −0.1915 | −0.1735 | −0.0545 | −0.5375 | 0.2355 | 0.1545 | −0.2055 | −0.3555 |
| AGT | 0.348 | −0.378 | −0.221 | −0.126 | −0.447 | 0.185 | 0.306 | 0.218 | −0.187 | −0.289 | −0.358 | 0.406 | −0.444 | −0.121 | −0.776 |
| AGTR1 | 0.079 | −0.499 | 0.254 | −0.213 | −1.042 | 0.782 | 1.45 | −0.476 | 1.176 | −0.42 | 0.423 | 1.057 | −0.821 | −1.031 | −0.003 |
| AHCYL1 | −1.614 | −0.575 | 0.068 | −0.222 | −0.474 | 0.939 | 0.538 | 0.426 | 0.222 | 0.114 | 0.42 | −0.752 | −0.438 | 1.804 | −0.147 |
| AHNAK | 0.574 | 0.478 | −0.046 | −0.225 | 0.491 | 0.291 | 0.817 | 1.443 | 0.982 | −0.589 | 0.315 | −0.382 | −0.711 | −1.048 | 0.755 |
| AHR | 0.6065 | 0.3435 | −0.0675 | 0.5055 | −0.0125 | −0.2825 | −0.0285 | −0.2185 | −0.1485 | −0.0225 | 0.8095 | −0.4815 | −0.0435 | 0.7155 | 0.1185 |
| AIM1 | −0.8865 | 0.4615 | −0.3385 | −0.3235 | −0.0405 | 0.0005 | −0.1525 | 0.0995 | −0.6025 | −0.8595 | 0.0325 | −0.4875 | −1.0075 | 0.5665 | −0.0765 |
| AK2 | −0.7885 | −0.8925 | −0.0625 | −0.3545 | −0.7765 | −1.5025 | 0.0325 | 2.7525 | 0.4295 | 2.5265 | −0.7615 | −0.2115 | 0.0575 | 1.5055 | −1.2615 |
| AK3L1 | −0.938 | 1.266 | −0.98 | 0.458 | −1.442 | −0.326 | 0.225 | 0.349 | 0.097 | 0.282 | −0.025 | 0.527 | −0.026 | −0.702 | 0.328 |
| AK5 | 2.019 | −0.208 | 0.473 | 0.378 | 0.035 | 0.659 | 0.521 | 0.138 | −0.094 | −0.001 | −0.279 | 0.276 | −0.188 | −0.182 | 0.171 |
| AKAP1 | 0.0316 | −0.5135 | −0.3276 | −0.4715 | −0.2795 | −0.8625 | −1.0775 | −0.3795 | −1.0515 | 0.1545 | −1.0475 | −0.3455 | −0.6205 | −0.4575 | −0.1855 |
| AKR1A1 | −0.963 | 0.349 | −0.323 | 0.171 | 0.193 | 0.133 | 0.013 | −0.795 | −0.227 | −0.252 | −0.599 | −0.323 | −0.964 | −0.722 | 0.377 |
| AKR7A3 | 0.2995 | 0.3135 | 0.1615 | 0.0885 | −0.0985 | −0.2365 | −0.2745 | −0.1865 | 0.6815 | −0.6675 | −0.7455 | −0.3005 | −0.3615 | 0.0555 | −0.0505 |
| ALG5 | 0.0775 | −0.7945 | 0.1155 | −1.5935 | −0.6835 | −1.8985 | 0.5485 | −2.0455 | 0.5455 | −0.7925 | −0.2505 | −1.4335 | −0.0025 | −0.2475 | 0.3975 |
| ALG8 | 0.1335 | −0.3255 | 0.1505 | −0.2285 | −0.6965 | −0.0855 | 0.6035 | 0.4325 | 0.2955 | 0.1395 | −0.0715 | 0.5875 | 1.2565 | 0.1325 | 0.3825 |
| AMFR | 0.402 | −0.575 | −0.515 | −0.084 | −0.423 | 0.134 | −0.118 | 0.121 | −0.29 | −0.542 | −0.995 | −0.26 | −0.956 | −0.327 | −0.174 |
| AMPD2 | 1.077 | −0.63 | 0.554 | 1.315 | 0.811 | 0.313 | 1.818 | 2.645 | −0.819 | 0.599 | −0.381 | 0.207 | −0.965 | 1.102 | −0.813 |
| ANAPC11 | −0.513 | −0.491 | −0.67 | 0.286 | 0.812 | 0.059 | 0.446 | 0.418 | 0.087 | 0.427 | 0.701 | −0.078 | 0.335 | 1.362 | −0.484 |
| ANKRD22 | 0.337 | 0.868 | 0.475 | 1.568 | 0.626 | −0.187 | 0.355 | −0.431 | −0.118 | −0.177 | 1.795 | 0.929 | 0.251 | 1.194 | 0.941 |
| ANKRD27 | 0.093 | −0.136 | 0.275 | −0.348 | 0.813 | 0.013 | −0.935 | −0.4 | −0.235 | −0.352 | −0.273 | −0.56 | −0.171 | 0.172 | 0.415 |
| ANLN | −1.403 | 0.597 | 1.624 | 1.917 | 1.563 | 0.688 | 2.149 | −1.643 | −1.285 | 1.113 | 1.052 | 1.312 | −0.388 | −0.683 | −0.333 |
| ANP32A | −0.127 | −0.673 | −0.208 | −0.721 | 1.278 | −0.545 | −0.803 | 0.171 | 0.529 | −0.43 | −0.796 | −0.642 | −0.365 | −1.222 | −1.048 |
| ANXA3 | −0.3065 | −2.3705 | 0.2345 | 1.3975 | −0.4445 | 0.6655 | −2.7005 | −0.2735 | −1.0915 | 0.0375 | 1.1555 | 2.0795 | 0.9615 | 0.7885 | −1.2355 |
| APBB2 | −0.9965 | −0.8395 | 1.1125 | 1.0585 | −0.8835 | −1.2855 | 0.7845 | −0.0885 | 1.0555 | −0.7925 | 2.2355 | 4.2455 | 2.3765 | 0.6595 | −0.7425 |
| APOBEC3 | 0.017 | 0.017 | 0.106 | −1.026 | 0.404 | −0.046 | −0.928 | 0.163 | 0.299 | −1.578 | −4.107 | −3.671 | −4.555 | −5.258 | −4.762 |
| AQP5 | −1.634 | −1.659 | −0.984 | 1.498 | −1.455 | −1.024 | −0.07 | 0.924 | −0.412 | −1.35 | −1.705 | −0.879 | −0.442 | −1.109 | −1.012 |
| AR | −0.0785 | −0.0955 | 0.6065 | −0.1615 | 0.3535 | 0.1015 | 0.1625 | −0.3495 | −0.1435 | 0.2135 | 0.3125 | −0.3315 | 0.7385 | −0.1005 | −0.1305 |
| AREG | 0.149 | −0.069 | −0.045 | −0.517 | 0.541 | 0.9 | 0.1 | 0.981 | 0.294 | −0.52 | −0.504 | 0.496 | −0.067 | −0.923 | −1.834 |
| ARF1 | −0.4435 | −0.6595 | −0.2585 | 0.1745 | 0.7945 | −0.1885 | −0.4785 | −0.1495 | 0.4755 | −0.1825 | −0.0405 | −0.1125 | −1.2075 | −0.1585 | 0.0105 |
| ARHGDI [symbol] | −0.342 | −0.424 | −0.479 | 0.633 | 0.217 | −0.299 | 0.638 | −0.268 | −0.397 | −1.178 | 0.927 | 1.256 | 0.616 | 1.418 | 0.872 |
| ARL6IP | −1.5555 | 0.1105 | −0.4275 | −0.8685 | 0.5385 | −0.3755 | −1.5465 | −0.5005 | −0.4825 | −0.2675 | −1.1665 | −1.0105 | −0.3595 | 0.2715 | −0.7895 |
| ARL6IP2 | −2.281 | −1.633 | −1.629 | −1.229 | −1.523 | −0.953 | 0.411 | −0.697 | −0.301 | −2.272 | −2.107 | 1.31 | −0.81 | −0.8 | 1.198 |
| ARMCX2 | −2.293 | −1.73 | −1.715 | −1.433 | −1.471 | −2.19 | 0.31 | −0.564 | −0.164 | −1.724 | −2.173 | 1.057 | −0.296 | −1.053 | 1.077 |
| ARNT2 | −0.659 | −0.344 | −0.958 | 0.389 | −0.176 | −0.522 | −0.369 | 0.068 | 0.158 | −0.136 | 0.074 | 0.836 | 0.811 | 1.418 | 2.864 |
| ARNT2 | 1.2885 | 0.3085 | 0.8835 | 1.9545 | −0.0635 | 0.7395 | 2.3055 | 0.7655 | −0.1135 | 1.1555 | 1.2465 | 0.7895 | 0.4345 | 0.3205 | −0.1185 |
| ARP3BET [symbol] | −0.3185 | −0.1415 | 0.2195 | 0.7395 | 0.3365 | 0.5585 | 0.4195 | −0.0045 | −0.3825 | −0.3065 | −0.1335 | −0.2905 | −0.0116 | 0.2865 | 0.0095 |
| ARPC1B | −0.221 | −0.005 | −0.056 | 1.658 | 1.781 | −0.708 | 0.005 | 0.013 | −0.195 | −0.093 | −0.242 | 0.014 | −0.106 | 0.079 | 0.162 |
| ARPC5L | 0.1205 | 0.4735 | 0.3555 | −0.2515 | 0.0895 | 0.4575 | −0.8585 | −0.1585 | 0.0205 | −0.8505 | 0.2045 | −0.1275 | −1.3225 | 0.4605 | 1.3115 |
| AS89 | −0.531 | −0.386 | −0.183 | −0.593 | 0.13 | −0.272 | −0.217 | 1.158 | −0.353 | 0.65 | 0.257 | 0.075 | −0.806 | 0.167 | 0.014 |
| ASF1A | 0.5665 | 0.2135 | −0.3115 | 0.7185 | 0.2195 | 0.0595 | 1.0835 | −0.3835 | 0.3225 | −0.3625 | 0.3995 | −0.3225 | −0.6575 | 0.0726 | 0.9825 |
| ASH2L | −0.652 | 1.196 | −0.096 | −0.304 | 0.582 | 1.812 | −0.08 | −0.058 | −0.082 | 0.575 | 1.13 | 0.702 | 0.374 | 1.65 | 1.142 |
| ASK | 0.745 | 0.963 | 0.818 | 1.871 | −0.458 | 0.37 | 0.86 | 0.193 | 0.27 | 0.189 | 1.803 | 0.573 | 0.753 | 1.411 | −0.343 |
| ASPM | −0.5315 | 1.2925 | 0.4485 | 0.8805 | 0.7935 | 0.0385 | −0.6595 | −0.4785 | −0.0055 | −0.5795 | 0.6005 | 0.6855 | −0.4455 | 0.5245 | 0.1745 |
| ASS | 0.5885 | −1.0685 | −0.1085 | −0.7015 | −0.2325 | 0.0475 | 0.7025 | 0.7065 | 1.6405 | 0.3465 | 0.5005 | 0.6305 | −0.0555 | −0.4225 | 0.5555 |
| ATAD2 | −0.0015 | −0.5555 | 0.5965 | 0.0825 | −0.4585 | 0.1116 | −0.6035 | 1.0605 | 0.4415 | −0.4535 | 0.7665 | 0.4775 | −0.8045 | 0.2015 | 0.8635 |
| ATF5 | 2.159 | −0.098 | 0.005 | 0.175 | 1.018 | 0.238 | 0.222 | 0.186 | −0.81 | −0.285 | −0.948 | −0.523 | −1.052 | −0.351 | 0.334 |
| ATF7IP2 | 3.37 | 2.208 | −0.131 | 0.503 | 0.109 | 0.022 | −0.283 | 0.124 | −0.044 | −0.872 | −0.182 | 0.093 | −0.677 | −0.008 | 0.88 |
| ATP2B1 | 0.648 | −0.704 | 0.132 | 0.34 | 0.021 | 0.133 | −0.446 | −0.115 | −0.58 | −0.146 | −0.571 | 0.245 | −0.711 | −0.194 | 0.774 |
| ATP5G1 | 0.3155 | −0.6485 | −0.0725 | −0.1255 | 0.4565 | 0.4215 | 0.3875 | 0.0346 | 0.4125 | −0.8165 | −0.5585 | 0.1935 | −0.0245 | −1.3935 | −0.9785 |
| ATP5O | 0.25 | −0.178 | 0.012 | 1.823 | −0.33 | −0.223 | 2.595 | −0.355 | 0.877 | 0.851 | −0.616 | 0.477 | 0.278 | 1.039 | 1.333 |
| ATP6AP1 | 0.012 | −0.175 | 0.744 | 0.642 | 0.936 | 0.142 | 0.815 | 0.785 | 0.196 | −1.178 | 0.723 | 0.068 | −0.47 | 0.347 | 1.293 |
| ATP6V0A4 | −1.209 | −0.544 | 0.187 | 0.262 | 0.157 | −0.029 | −0.332 | −0.513 | −0.63 | −0.897 | −1.531 | −0.827 | −0.53 | −0.918 | −1.082 |
| ATP6V1B [symbol] | 0.3705 | 0.4625 | 0.3695 | −0.2555 | 0.0825 | 0.0515 | −0.4505 | −0.4975 | 0.2255 | −0.0525 | −0.5825 | −0.9535 | −0.2395 | −0.8745 | −0.9835 |
| ATP6V1G [symbol] | −0.639 | 0.35 | 0.198 | −0.155 | 0.062 | −0.192 | −0.11 | −0.081 | −0.873 | −0.202 | −0.214 | 0.041 | −0.926 | −0.061 | 0.013 |
| ATPIF1 | 0.1065 | −1.4125 | 0.8525 | 2.4165 | −0.7305 | 0.2235 | 1.3795 | 0.1535 | −1.1085 | 0.1135 | 1.1645 | 1.3135 | 1.0085 | 2.8845 | 1.6445 |
| ATXN10 | 0.961 | −0.163 | 0.861 | 1.143 | −0.044 | 2.387 | 1.126 | −0.146 | −0.032 | 0.588 | 0.9 | 1.217 | 0.963 | −1.084 | 1.688 |
| B3GNT5 | −0.3895 | −0.4415 | −0.7885 | −0.0675 | −1.7215 | −0.3055 | −0.3825 | −0.5175 | −0.9145 | −0.6355 | 0.2105 | −0.8835 | −0.7355 | −1.0185 | 0.5885 |
| BACE2 | −0.2375 | −0.1295 | −0.2345 | −0.4525 | −0.2015 | −0.1585 | −0.4685 | 0.2705 | −0.2155 | 0.0835 | 0.1825 | −0.5415 | −1.0705 | 0.2165 | −0.1685 |
| BAG1 | −0.4435 | −0.4875 | 0.8535 | −0.7845 | 0.0845 | −0.9445 | −0.3515 | −0.3345 | 1.0085 | −0.2935 | −0.1745 | −0.9555 | 0.1965 | −0.7785 | −1.0395 |
| BAG4 | −0.9155 | −0.5545 | 0.0945 | 1.3285 | 2.4205 | 0.0685 | −0.3755 | 0.0065 | 1.0475 | 3.1725 | −0.7755 | −0.4855 | 0.4625 | −0.4395 | 0.1115 |
| BAI2 | 0.718 | 0.735 | 0.476 | 0.955 | 0.481 | 1.097 | 0.317 | −0.781 | −0.185 | 1.825 | −0.534 | −2.098 | −0.549 | −0.923 | −1.179 |
| BAMBI | 0.099 | 0.097 | −0.295 | −0.332 | 0.664 | −0.220 | 1.239 | 1.274 | 0.17 | −0.296 | 0.339 | −0.004 | −0.022 | 1.192 | 0.728 |
| BCAS1 | −4.89 | −5.037 | −1.438 | −3.415 | −1.864 | −4.811 | −4.156 | 0.597 | −0.219 | −0.751 | −3.978 | −3.106 | −5.163 | −4.834 | −4.534 |
| BCL2L14 | −1.0985 | −1.0325 | −1.0655 | −1.0205 | −0.5475 | −1.1015 | −0.5185 | −0.1925 | 2.3625 | −0.7935 | −0.5165 | −1.1255 | −0.4125 | 1.2295 | −0.2195 |
| BCMP11 | −2.357 | −0.528 | −0.814 | −0.773 | −0.028 | −0.668 | −0.533 | −0.726 | 0.901 | −1.182 | −0.013 | −1.002 | −0.455 | 1.748 | −0.336 |
| BEX1 | −1.8365 | −3.2075 | 0.3035 | −2.2165 | −0.6235 | −0.5005 | 0.5475 | −0.2325 | −0.0025 | 0.5875 | −0.9835 | −0.7048 | −1.4275 | −0.0735 | −1.2635 |
| BEX2 | 0.7285 | 1.1515 | −0.1005 | 1.2735 | 0.1916 | 0.8395 | 0.2955 | 0.1115 | 0.8095 | 1.4195 | −0.2415 | 0.0715 | 0.5395 | −0.2975 | 0.4705 |
| BF | −0.046 | 0.313 | −0.32 | −0.072 | −0.204 | 0.068 | 0.14 | 0.053 | −0.421 | −0.621 | −0.421 | 1.751 | −0.415 | −0.327 | −0.573 |
| BFSP1 | −0.693 | −0.094 | 0.56 | 0.231 | 0.173 | 0.414 | −0.059 | 0.182 | 0.143 | −0.318 | 0.947 | 0.487 | 0.331 | 0.053 | 1.39 |
| BIK | 4.825 | 1.494 | 0.027 | 0.8 | −0.048 | −0.216 | 0.537 | 0.349 | −0.336 | 0.579 | 1.428 | 0.611 | −0.027 | 0.879 | 1.352 |
| BIRC2 | −0.2485 | 1.1945 | 0.5085 | 2.6535 | −0.1015 | −0.6225 | 0.5405 | −0.0345 | −0.2415 | 0.4905 | −0.1375 | −0.3975 | −0.4855 | 0.2165 | 0.8555 |
| BIRC5 | −0.2655 | 0.4625 | 0.4535 | −0.0615 | −0.2725 | −0.2685 | −0.6195 | −1.5195 | −0.3485 | −0.6555 | 0.5385 | −0.7335 | 0.1155 | 0.3545 | 0.6135 |
| Bit1 | 1.1105 | 0.3846 | 0.4505 | 0.5875 | −0.1445 | 0.2035 | 0.2085 | −0.3495 | 0.1855 | 0.8295 | −0.8725 | −0.5165 | −0.7545 | −1.0575 | −0.1565 |
| BIVM | −0.742 | 0.429 | 0.017 | 0.8 | 1.061 | −0.855 | −0.332 | −0.36 | −0.129 | 0.602 | 0.896 | 0.686 | −0.03 | 1.471 | 1.481 |
| BLVRA | 1.868 | −0.166 | −0.269 | 2.684 | 0.462 | 0.453 | −1.323 | 2.584 | −0.442 | 0.526 | 0.081 | −0.001 | 1.944 | 1.048 | 0.312 |
| BM039 | −0.038 | −0.265 | −0.175 | −0.731 | −0.672 | −0.024 | −0.222 | 0.414 | −0.164 | 0.232 | 0.848 | 0.294 | 0.556 | −0.268 | 0.027 |
| BMP7 | 0.426 | −0.042 | 1.192 | 0.122 | 0.093 | −0.647 | 0.175 | 0.31 | −0.064 | −0.121 | 0.503 | 0.364 | −0.354 | 0.566 | 0.588 |
| BOC | −0.8565 | −1.0285 | −0.3435 | −0.9195 | 0.1395 | −0.2735 | −0.0385 | 2.2555 | −0.6485 | 0.3785 | −0.0475 | 0.2205 | −0.6605 | −0.0145 | 0.3325 |
| BRCA1 | 0.6335 | −0.2905 | −0.0005 | 0.5195 | 0.2405 | −0.0385 | 0.8915 | −0.0495 | −0.1855 | 0.4055 | −0.1805 | 0.4585 | 0.6685 | 0.1305 | 0.4125 |
| BRF2 | 0.709 | −0.221 | 0.159 | −0.039 | −1.105 | −0.507 | 0.8 | −0.227 | −0.058 | −0.8 | 1.205 | 0.602 | 0.04 | 0.163 | 0.617 |
| BRI3 | −0.288 | −0.348 | 0.045 | 1.437 | −0.58 | −0.788 | −0.328 | 0.258 | −0.182 | 0.54 | −0.287 | −0.981 | 0.545 | −0.458 | −1.656 |
| BRI3BP | −0.873 | −0.41 | −0.844 | −0.687 | −0.393 | −0.721 | −1.016 | −0.593 | −0.567 | −0.986 | −0.755 | −0.598 | −1.379 | −0.681 | −0.261 |
| BSPRY | 0.3515 | −0.1125 | 0.9295 | 0.8945 | −0.7275 | 0.6145 | 0.1925 | 0.1535 | −0.4415 | −1.5275 | 0.4575 | 0.2775 | 0.6305 | 1.0105 | 1.8135 |
| BTF3 | −0.0555 | −0.3415 | −0.0855 | −0.5335 | 1.1335 | 0.7045 | 0.2895 | 1.0825 | 0.1405 | 0.8985 | 0.1945 | 0.9665 | 0.5465 | −0.3715 | −0.4225 |
| BTG3 | −0.233 | 0.479 | 0.04 | 0.309 | 0.720 | 0.023 | 0.302 | −0.315 | −0.298 | −0.123 | 1.161 | 0.684 | −0.023 | 1.12 | 1.015 |
| BγN2A2 | −0.831 | 0.23 | −0.318 | 0.146 | 0.619 | −0.194 | −1.05 | −0.659 | −1.062 | −0.196 | −0.765 | −0.984 | −1.405 | −0.912 | 0.225 |
| BUB1 | 0.7135 | 0.8805 | −0.2185 | −0.8245 | −0.7375 | −0.2855 | −0.1995 | 0.0045 | 0.0045 | 0.1925 | 0.6405 | 0.5415 | −0.0995 | 0.6425 | 1.9845 |
| BUB3 | 1.049 | 0.773 | 0.172 | 0.382 | −0.217 | 0.191 | 0.755 | 0.439 | −0.281 | 0.645 | 0.776 | 0.936 | 0.968 | −0.401 | −0.034 |
| BXDC1 | −0.378 | −0.33 | 0.283 | 0.911 | 0.286 | 0.076 | −0.201 | −0.816 | −0.857 | −0.254 | −1.097 | −0.71 | −1.31 | −0.717 | 0.055 |
| BZRP | −0.867 | −2.974 | −0.931 | −1.91 | −2.421 | −1.883 | −0.372 | 0.717 | −0.054 | 0.011 | −1.255 | 0.33 | −2.514 | −2.938 | −0.968 |
| BZW1 | −0.6035 | 0.0395 | −0.4825 | 0.6315 | −0.4055 | −0.0005 | −0.6215 | 0.5445 | 0.1585 | 0.1335 | 0.4605 | 1.3195 | 1.9555 | 1.4955 | 2.1045 |
| C10orf116 | −0.3075 | 0.3075 | −0.7675 | −0.8795 | −0.5745 | 0.1805 | −0.7935 | −0.0195 | −0.9235 | −0.1835 | −0.3105 | 0.3905 | −0.6965 | −0.1795 | −0.4635 |
| C10orf38 | 0.508 | −0.663 | 0.852 | 1.325 | −0.414 | 0.981 | 1.7 | 1.443 | −0.428 | 1.73 | −0.742 | −0.038 | −0.099 | −0.892 | 2.081 |
| C10orf4 | −0.0795 | 0.0385 | 0.2275 | 0.7195 | −0.3955 | −0.1175 | 0.5955 | −0.4925 | 0.6155 | 1.0575 | 0.4475 | 1.0585 | 0.4005 | 1.3595 | 2.0395 |
| C10orf45 | −0.3785 | 1.3455 | 0.4795 | −0.4965 | 0.5395 | −0.4585 | −0.9485 | 1.8445 | 0.8555 | 1.3515 | −0.4095 | 0.8925 | −0.5235 | −0.4755 | −0.6725 |
| C10orf7 | 0.3415 | −0.0585 | 0.5345 | −0.2395 | −0.7485 | 0.2745 | 1.1595 | 0.1815 | 0.0135 | −0.0065 | −0.2095 | 0.3565 | 0.4825 | −0.0415 | −0.0605 |
| C10orf82 | −0.733 | 0.322 | −0.125 | 0.128 | −0.068 | −0.4 | 0.721 | 0.568 | −0.003 | −0.071 | 1.271 | 2.306 | 1.292 | 2.001 | 1.339 |
| C11orf24 | −0.9855 | −0.3805 | 0.2235 | −0.8415 | −0.7255 | −0.5855 | 0.5155 | 0.0005 | −0.4155 | −0.9345 | −0.5875 | −0.5595 | 0.1185 | −0.5845 | −0.2455 |
| C12orf14 | −0.662 | 0.291 | 0.249 | 0.748 | 0.178 | 0.312 | 1.082 | −0.123 | −0.243 | 0.309 | 0.548 | 0.513 | 0.017 | 1.372 | 0.63 |
| C13orf21 | −0.265 | 0.687 | −0.421 | 0.579 | −0.003 | −0.117 | 0.027 | −0.026 | −0.012 | 1.993 | 0.415 | −0.113 | −0.301 | −0.628 | −0.348 |
| C13orf23 | 0.211 | 0.172 | −0.321 | −1.055 | −0.475 | −0.531 | −0.289 | −0.5 | −0.398 | −0.504 | 0.784 | 0.078 | −0.773 | 0.301 | 0.883 |
| C14orf94 | −0.39 | 0.01 | 0.73 | −0.76 | −0.78 | −0.64 | −0.53 | −0.52 | −0.295 | −0.44 | −0.91 | −0.32 | −0.626 | −1.603 | −0.813 |
| C15orf23 | 2.9915 | 1.9315 | 2.1555 | 0.9135 | 2.8775 | 3.1125 | 1.9005 | 0.1875 | −0.4815 | 1.3735 | 0.0245 | 0.9945 | 0.8055 | −0.8005 | −0.6975 |
| C16orf45 | −0.665 | 0.738 | 0.678 | −0.631 | 0.943 | −0.048 | −0.426 | 1.365 | 0.12 | −0.133 | −0.173 | 0.155 | 0.05 | −1.74 | −0.975 |
| C17orf37 | 0.483 | −0.577 | 0.158 | 0.481 | 0.141 | 0.27 | 0.305 | 0.112 | −0.448 | 0.462 | −0.288 | −0.134 | 0.005 | −0.29 | 0.408 |
| C18orf1 | 2.592 | 0.056 | 0.285 | 0.949 | −0.818 | 0.133 | −1.39 | −0.008 | 0.707 | 0.645 | −1.458 | −1.669 | −1.088 | −1.424 | −1.985 |
| C19orf10 | −0.2305 | −0.0755 | 0.2655 | 1.5485 | 0.6055 | −0.9045 | 2.1415 | −0.3325 | −0.0645 | 0.6215 | 0.2895 | −0.4105 | −0.5515 | −0.2285 | −0.7195 |
| C19orf32 | −0.0775 | −2.3205 | −1.9915 | −1.4365 | −1.7495 | −0.0335 | 0.1465 | 0.1465 | −1.6635 | −2.3225 | −0.9735 | 0.4945 | −0.2215 | −1.2125 | −1.9405 |
| C19orf33 | 0.18 | −2.027 | −0.544 | 0.828 | 0.021 | −0.125 | −0.887 | −0.164 | 1.374 | 1.302 | −1.034 | −2.064 | −0.734 | −0.898 | −2.037 |
| C1orf24 | −1.201 | −0.301 | 0.335 | −0.88 | −1.138 | 1.203 | 1.246 | 0.774 | −0.313 | −0.538 | −1.001 | −0.131 | −0.325 | −1.576 | −1.513 |
| C1orf34 | 1.0995 | 0.0665 | −0.0955 | −0.4235 | 0.2625 | 0.7325 | −0.1325 | 1.2215 | 0.1025 | 0.0215 | 0.2055 | −0.2675 | −0.4845 | −0.8095 | −0.8725 |
| C20orf103 | −0.8135 | −0.2255 | 0.0115 | −0.5225 | −0.5075 | 0.2575 | −0.6845 | −0.4585 | 0.2895 | −0.1695 | −0.5285 | −1.7285 | −0.5835 | −1.9355 | 0.0585 |
| C20orf149 | 2.501 | −0.117 | 0.814 | 0.207 | 1.233 | 0.761 | −0.688 | 0.252 | 0.144 | 0.5 | −0.755 | −0.549 | −0.106 | −1.01 | −0.072 |
| C20orf23 | −0.69 | −0.955 | −0.778 | −0.028 | −1.003 | −1.014 | −0.562 | −0.558 | −0.143 | −0.255 | 0.268 | 0.352 | −0.255 | −0.759 | 0.191 |
| C20orf3 | 0.407 | −0.225 | −0.151 | 0.074 | 0.272 | 0.123 | −0.345 | −0.148 | −0.156 | 0.127 | 0.175 | 0.096 | −0.275 | 0.074 | 0.281 |
| C20orf35 | −0.003 | −0.125 | −0.095 | −0.334 | −0.307 | 0.813 | −0.398 | 0.05 | −0.033 | −1.13 | 0.483 | −0.866 | 0.465 | 0.212 | 0.975 |
| C20orf43 | 0.5855 | 0.0445 | 0.3295 | 0.4755 | 0.3155 | 0.0205 | −1.1555 | −0.1255 | −0.4395 | −0.2195 | −0.2415 | −0.0255 | 0.0475 | 0.5585 | 0.6995 |
| C20orf8 | 0.807 | 0.056 | −0.373 | −0.361 | −0.4 | −0.058 | 0.557 | 0.548 | −0.275 | −0.353 | 1.058 | 0.837 | 0.223 | 0.76 | 0.126 |
| C21orf45 | 0.132 | 0.028 | −0.325 | −0.159 | −0.139 | −0.418 | −0.135 | 0.389 | 0.159 | −0.242 | 0.842 | 1.147 | 0.374 | 1.689 | 2.236 |
| C22orf18 | 0.25 | −0.318 | −0.021 | −0.166 | −0.256 | −0.168 | −0.484 | 0.203 | −0.185 | 0.489 | −0.35 | 0.488 | 0.018 | −0.62 | 0.458 |
| C2F | −0.433 | −0.858 | −0.068 | −1.577 | −1.52 | −0.128 | −0.723 | 0.398 | 1.105 | −0.116 | −1.397 | −0.259 | −0.335 | −0.521 | −0.021 |
| C2orf17 | −0.068 | 0.717 | 1.191 | 0.557 | 1.124 | −0.088 | −0.075 | −0.235 | −0.268 | 0.396 | 0.124 | −0.408 | −0.602 | −0.068 | −0.629 |
| C4A | 0.58 | 0.194 | −0.56 | −0.43 | −0.49 | −0.215 | −0.344 | 0.199 | 0.459 | −0.1 | 0.392 | 1.101 | 0.488 | 0.155 | 0.805 |
| C5orf13 | 0.506 | 0.165 | 0.381 | −0.14 | 0.691 | −0.095 | −0.21 | 0.232 | −0.374 | 0.454 | 0.262 | 0.249 | −0.471 | 0.201 | 0.112 |
| C6orf108 | 0.793 | 0.274 | 0.47 | −0.098 | −0.511 | 0.085 | −0.663 | 0.079 | −0.036 | 0.332 | 0.009 | −0.534 | −0.22 | 0.13 | 1.022 |
| C6orf166 | −0.4 | −1.317 | −0.77 | −0.803 | −0.395 | −0.702 | −1.819 | 0.908 | 0.879 | −0.471 | −1.624 | −0.871 | −2.471 | −1.752 | −0.875 |
| C6orf192 | 3.7255 | 0.3635 | −0.0075 | −0.5315 | −0.3185 | 0.0975 | 0.0385 | 0.1815 | 0.0225 | 0.1055 | 0.1475 | 0.5225 | −0.4335 | 0.1805 | 0.3175 |
| C6orf211 | 0.463 | 0.935 | 1.497 | 1.274 | 1.538 | 1.113 | −0.177 | 0.721 | 1.178 | 0.877 | 0.227 | 0.535 | −0.625 | −0.528 | −0.356 |
| C6orf49 | 0.6905 | 0.9225 | 1.0215 | 1.1425 | 0.7235 | 1.5345 | 0.3075 | −0.0385 | 0.2425 | 0.5975 | −1.5095 | −0.6775 | 0.2585 | 0.2375 | 0.0075 |
| C6orf85 | −1.2485 | −0.5705 | −0.6635 | −0.8965 | −0.8465 | −0.5955 | 0.4975 | 0.4055 | −0.1955 | −0.0045 | −0.5215 | −0.0155 | 0.3915 | −0.4055 | −0.0135 |
| C7orf24 | −1.227 | −0.514 | −0.014 | −0.506 | −0.15 | −1.23 | −1.287 | −0.973 | 1.767 | 0.173 | −0.248 | −0.594 | −0.707 | −1.681 | −0.917 |
| C9orf10 | 0.887 | −1.045 | 0.299 | 0.495 | 0.706 | 0.631 | 0.691 | 0.376 | −0.697 | −1.183 | 0.708 | 1.097 | 2.188 | −0.017 | 0.771 |
| C9orf121 | 0.439 | 0.079 | 0.174 | −0.073 | −0.266 | 0.351 | 0.468 | 0.332 | −0.008 | 0.059 | 0.45 | 0.17 | 0.27 | 0.501 | −0.132 |
| C9orf58 | −0.5135 | 0.0325 | −0.3045 | −0.4195 | 0.1145 | −0.2615 | 0.3715 | −0.2215 | −0.3705 | −0.6655 | −0.1285 | 0.1155 | 0.7305 | −0.1335 | 0.1275 |
| C9orf88 | 0.686 | −1.184 | −0.645 | −1.713 | −1.837 | −1.2 | −2.231 | 1.149 | 1.051 | −0.015 | −1.974 | −1.532 | −2.584 | −1.887 | −0.317 |
| C9orf9 | −0.26 | −0.211 | −0.008 | −0.83 | −1.979 | −0.176 | −2.288 | 0.955 | −0.396 | −0.789 | −0.028 | 0.767 | 1.498 | −0.686 | 0.116 |
| CA12 | −0.328 | −0.482 | −0.075 | −0.71 | −2.088 | −0.049 | −2.571 | 0.937 | −0.851 | −1.181 | −0.039 | 0.853 | 1.056 | −0.967 | 0.263 |
| CA8 | −0.386 | −0.365 | −0.481 | −0.042 | −0.299 | −0.317 | 0.101 | −0.088 | −0.094 | 0.141 | −0.295 | −0.01 | −0.15 | −0.251 | −0.276 |
| CA8 | 0.0505 | −0.0305 | −0.1145 | −0.0025 | 0.0625 | 0.1945 | 0.0525 | −0.1555 | −0.1455 | −0.0725 | 0.2035 | −0.0015 | −0.1785 | 0.4235 | 0.9165 |
| CACNA1G | 0.188 | −0.018 | 0.326 | −0.568 | 0.33 | −0.121 | −0.015 | 0.855 | 0.193 | 1.105 | 0.099 | 0.198 | 1.528 | −0.098 | 0.133 |
| CALCA | 2.938 | 1.953 | 2.575 | 1.252 | −0.159 | 0.309 | 4.64 | −0.138 | 0.404 | −0.261 | 2.175 | 4.141 | 0.272 | −0.394 | 1.961 |
| CALML4 | −1.2085 | 1.2595 | 0.8315 | −0.4015 | −0.1135 | −0.4395 | 1.0935 | −0.0065 | 0.9135 | −0.0455 | −1.0115 | −1.7705 | 0.1515 | −1.4875 | −1.0905 |
| CALML5 | −0.9415 | −0.3005 | −0.1555 | −1.0295 | −0.1715 | 0.3175 | −0.9305 | −0.0285 | 0.8545 | 0.3825 | 0.2295 | 0.8795 | −0.1195 | −0.2085 | −1.2885 |
| CaMKIINa [symbol] | −1.751 | 0.435 | −0.009 | −0.107 | 0.497 | 0.097 | −0.128 | 0.182 | −0.295 | 0.003 | −0.051 | 0.037 | −0.381 | −0.426 | −0.014 |
| CAP2 | 1.447 | 0.707 | 1.191 | 0.068 | 0.305 | −0.682 | 0.295 | 1.705 | −0.619 | −0.079 | −1.275 | 2.044 | −0.395 | −0.981 | −0.899 |
| CAP350 | −0.1745 | −0.2005 | −0.1275 | −0.0175 | −0.2405 | −0.1475 | −1.5235 | 0.3375 | 0.0175 | −0.4415 | 0.2995 | −0.2275 | 0.0705 | 0.3495 | −0.3055 |
| CAPN13 | 2.04 | −0.421 | 0.577 | −0.219 | −0.4 | −0.459 | 0.657 | 0.724 | 1.412 | −0.929 | 0.427 | 1.677 | −0.317 | −0.613 | −0.608 |
| CAPN9 | −0.1185 | −0.5075 | −0.4805 | −0.2945 | −0.1735 | −0.4315 | −0.1575 | −0.4155 | −0.2385 | 0.0185 | −0.4835 | −0.3245 | −0.3645 | −0.1025 | −0.1975 |
| CARD14 | 0.2515 | −0.4945 | 0.6285 | 0.0835 | 0.4495 | 0.7795 | 0.8805 | 0.9645 | −0.8645 | −0.4115 | −0.2795 | 0.1085 | −0.4205 | 0.7375 | 0.2055 |
| CART | 0.6625 | −0.0245 | 0.4455 | 0.4215 | 0.9755 | 0.7055 | 0.5155 | 0.2645 | −0.3285 | −0.3225 | 0.4975 | 0.0405 | −0.7875 | −0.4765 | 0.0005 |
| CASP1 | −0.114 | 0.279 | 0.481 | −0.004 | 0.45 | 0.002 | −0.302 | −0.164 | −0.787 | 0.063 | −0.489 | −0.873 | −0.862 | −0.198 | −1.309 |
| CASP3 | 0.292 | 0.026 | 0.127 | −0.181 | 0.016 | −0.215 | 0.119 | 0.242 | −0.085 | −0.218 | −0.058 | 0.168 | 0.282 | −0.239 | −0.345 |
| CASP7 | 0.6545 | −1.1855 | 0.4445 | −0.9755 | −0.3755 | 1.8146 | −0.7155 | 0.0445 | −1.3815 | 1.0945 | 0.4745 | 0.6745 | 1.3885 | 0.8115 | 2.1025 |
| C9LN1 | 2.73 | −0.179 | 0.393 | 1.013 | −0.211 | 2.208 | −0.424 | −0.638 | −1.142 | 0.482 | 0.306 | 0.245 | 0.949 | −0.862 | 2.381 |
| CBR1 | 0.3515 | 0.6085 | 0.9285 | 0.6945 | 1.3285 | 0.0905 | −0.2585 | −0.3815 | −0.1935 | 0.6465 | −0.2035 | −0.7625 | −0.9205 | −0.7145 | 0.6895 |
| CBR3 | −0.014 | −0.148 | −0.282 | −0.413 | −0.317 | −0.109 | 0.009 | 0.267 | 0.040 | −0.009 | 0.09 | 0.394 | 0.256 | −0.157 | −0.572 |
| CBR4 | −0.9345 | −1.7525 | −0.0155 | 0.3785 | −1.5715 | 2.2445 | −2.2745 | −0.1625 | 0.8835 | 1.2355 | 0.2965 | −1.9285 | 2.2825 | 3.0925 | −1.9395 |
| CCL24 | 0.98 | 0.903 | 0.541 | 0.665 | 0.624 | −0.173 | 0.204 | −0.719 | −0.046 | 0.123 | 1.39 | −0.024 | 0.077 | 1.731 | 2.209 |
| CCL29 | −1.501 | −0.986 | −0.161 | −0.69 | −0.729 | −0.187 | 0.518 | −0.864 | −0.582 | −1.636 | 0.607 | 1.138 | −0.115 | −0.301 | 2.303 |
| CCNB2 | 0.3365 | −0.0005 | −0.4105 | 0.5085 | 0.1885 | −0.1895 | 0.2965 | 0.2405 | 0.2985 | 0.5395 | 1.0035 | 0.0335 | 0.1215 | 2.2875 | 0.4045 |
| CCND1 | −2.337 | −0.409 | 0.404 | −0.532 | 0.806 | −0.487 | −0.119 | 0.035 | −0.886 | 0.075 | −1.018 | −0.908 | −1.004 | −1.413 | −1.468 |
| CCNE1 | −0.8725 | −0.4225 | −0.5475 | −0.1355 | −0.0695 | −0.3545 | −1.2955 | −0.0285 | −0.8555 | −1.4365 | −0.7415 | −0.5785 | −1.1275 | −0.9235 | −0.8625 |
| CCNG2 | 0.428 | 0.056 | 0.051 | 0.257 | 1.749 | 2.785 | 1.74 | 0.562 | 0.256 | −0.238 | 0.531 | −0.937 | 0.041 | −0.445 | −1.144 |
| CCNH | 0.0405 | −0.2585 | −0.5165 | −0.1535 | 1.2705 | 1.9935 | 0.8615 | 2.9455 | 0.3295 | 0.2185 | 0.6545 | 0.1895 | 0.5175 | −1.0265 | −0.3875 |
| CD38 | −0.1885 | −0.0455 | −0.2305 | −0.5145 | 1.4185 | 1.5995 | 1.4385 | 0.4255 | 0.0055 | −0.0335 | 0.0875 | 0.2325 | 0.3395 | −0.4115 | −0.7035 |
| C030 | 0.1095 | 0.0885 | 0.2835 | 1.1215 | 0.4475 | 0.2255 | 0.3835 | −0.2325 | −0.4585 | −0.3285 | −0.1355 | 0.5385 | 0.4435 | −0.0215 | 0.3875 |
| CD4 | 0.3265 | −0.0705 | 0.9355 | 1.8385 | −0.7135 | −0.0515 | −1.1525 | −0.3365 | −0.7985 | 0.3425 | 1.0575 | 0.2505 | −0.0105 | 0.1485 | 1.5125 |
| CD58 | 0.103 | 0.649 | 0.528 | 0.502 | 0.715 | −0.475 | −0.859 | −0.817 | −0.354 | −1.274 | 1.547 | −0.436 | −0.845 | 0.804 | 1.139 |
| CD50 | 1.9705 | 0.3605 | −0.1615 | −0.0085 | −0.1235 | 0.8055 | 0.0715 | 0.0385 | −0.3855 | 1.2935 | 0.6575 | 0.6475 | −0.0485 | 0.6775 | 0.8055 |
| CDC2 | −0.8185 | 0.8555 | −0.8705 | 0.0935 | −0.0965 | −0.1455 | −0.2045 | 0.0155 | −0.3605 | −0.1485 | 1.0605 | 0.2755 | 0.4995 | 1.7235 | 1.2845 |
| CDC6 | 0.9365 | 0.5685 | 0.6875 | 1.6065 | 0.6875 | 0.0005 | 0.6555 | −0.0225 | 0.0205 | −0.0215 | 1.7015 | 1.1475 | −0.0855 | 1.8745 | 1.5615 |
| CDCA1 | −0.038 | −0.048 | 0.804 | 0.652 | 0.304 | 0.92 | 0.02 | 0.882 | 0.31 | −0.231 | 0.503 | 1.072 | 1.104 | 1.146 | 1.379 |
| CDCA5 | 1.131 | 0.291 | 1.037 | 0.752 | 2.127 | −0.487 | 0.886 | −0.276 | 0.558 | −0.386 | −0.008 | −0.085 | −0.358 | 0.048 | 0.065 |
| CDCA7 | −0.1145 | −0.0505 | 1.9225 | 1.7915 | −0.0345 | −0.8565 | 1.3305 | 0.3135 | −0.5095 | 1.0275 | 2.3115 | 1.5355 | 0.8676 | 1.4235 | −0.3275 |
| CDH1 | 1.835 | −0.598 | 0.046 | −0.283 | −0.526 | 0.031 | 0.22 | 0.056 | −0.022 | −0.378 | 1.297 | 0.77 | −0.381 | 0.639 | 0.374 |
| CDH3 | −0.0885 | −0.4015 | 0.1705 | −0.1495 | −0.5355 | −0.1195 | 0.1785 | 0.2245 | 1.1175 | −0.3015 | 1.0655 | −0.0625 | 0.6875 | 2.9355 | −0.3645 |
| CDK2 | −0.1755 | −0.6135 | −0.1545 | 0.0635 | 0.2375 | −0.3085 | −0.3465 | −0.1755 | −0.0035 | 0.7175 | 0.9955 | 0.1915 | 0.8285 | −0.3505 | 2.3945 |
| CDKN2A | 0.3425 | 0.8845 | 0.1655 | 1.1505 | 0.6155 | −0.0005 | 0.3705 | −0.5425 | −0.6765 | −0.3715 | 0.8135 | −0.2585 | −0.3665 | 0.2725 | −0.1895 |
| CDKN2D | 0.278 | 1.224 | −0.395 | −0.625 | 0.556 | 0.341 | 0.328 | 0.076 | −0.222 | −0.679 | 1.456 | 1.418 | −0.214 | 1.379 | 0.83 |
| CDKN3 | −0.23 | −0.818 | −0.473 | 0.653 | −0.554 | 0.187 | −0.137 | 0.218 | −0.095 | 0.126 | 1.004 | −0.119 | 0.45 | 0.818 | 0.113 |
| CDT1 | −2.0595 | −1.3285 | −1.0795 | −1.4505 | −1.7235 | −0.9955 | 0.2655 | 0.2085 | 0.7335 | −0.5065 | −1.2055 | −0.0095 | −0.8385 | −0.6885 | −1.1065 |
| CE9PG | 0.452 | 0.375 | −0.584 | 0.979 | 0.637 | 0.161 | 0.278 | −0.398 | −0.43 | 0.295 | 0.975 | 0.255 | 0.434 | 1.543 | 0.729 |
| CELSR1 | 0.032 | 0.424 | 0.289 | 0.18 | 0.555 | −0.369 | −0.729 | −0.221 | −0.226 | 0.175 | 0.189 | 0.045 | 0.544 | 1.154 | 0.864 |
| CENPA | −0.145 | 1.468 | 0.782 | 0.158 | 1.141 | −0.661 | −0.138 | −0.33 | −0.072 | 0.466 | 1.274 | 0.579 | 0.873 | 1.936 | 1.177 |
| CENPE | 0.849 | −0.578 | −0.193 | −0.42 | −0.642 | −0.183 | 0.08 | 0.02 | 0.156 | −0.727 | 0.596 | 0.622 | 0.659 | 0.157 | 0.388 |
| CENPF | −0.5665 | −0.1365 | 0.1785 | −0.5815 | −0.2955 | 0.1675 | 0.0485 | 0.2265 | 0.2205 | −0.1815 | 0.2105 | −0.3735 | −0.1405 | −0.5855 | −0.4635 |
| CENTG3 | −1.3155 | −0.9245 | −0.4905 | −0.3325 | 0.0085 | −0.9805 | −1.4885 | −0.8885 | −0.8915 | −0.9515 | −1.0025 | −1.1225 | −1.4925 | −0.7985 | −0.6005 |
| CES2 | 0.876 | 0.758 | −0.54 | −0.319 | −0.287 | −0.486 | −0.809 | 0.797 | 0.283 | 0.544 | 0.915 | −1.167 | 0.669 | 0.081 | 2.897 |
| CETN3 | −0.9995 | −0.1235 | 0.3485 | 0.8535 | 0.0985 | −0.0035 | −0.0705 | 0.0425 | 0.2785 | −0.2615 | 0.2015 | 0.0075 | 0.5535 | 0.4435 | 0.8505 |
| CGA | −0.3585 | 1.7155 | −0.5945 | 1.0585 | 0.8095 | −0.0655 | 0.2925 | −0.0445 | −0.8375 | −0.8375 | −0.1965 | 0.0295 | −1.2785 | −0.2555 | 0.9935 |
| CGI-12 | −1.0645 | 2.1415 | 1.0435 | −1.2905 | −0.0335 | 0.2365 | −0.0105 | −0.0675 | 0.4555 | 1.1545 | −1.2735 | −0.5815 | −0.3185 | 0.0175 | 0.6885 |
| CGI-49 | −1.48 | −2.534 | −1.769 | −1.09 | −0.145 | −1.803 | 0.252 | 0.337 | 0.454 | 0.288 | −0.501 | 0.653 | −0.575 | 0.205 | −0.28 |
| CGI-4 [symbol] | 0.854 | −2.397 | −0.374 | −0.109 | −1.57 | 0.884 | 0.041 | −0.285 | −1.094 | −0.256 | −1.329 | 0.362 | −1.025 | −1.77 | 1.151 |
| CGN | 1.521 | −0.071 | 0.115 | 0.013 | 0.082 | 0.386 | −1.219 | −0.287 | −0.387 | 0.779 | 0.637 | −0.058 | −0.267 | 0.083 | 1.323 |
| CGNL1 | 0.4875 | 0.9185 | 0.3735 | −0.1805 | −0.7935 | 0.3835 | 0.6155 | −0.0355 | −0.3335 | 0.9225 | 1.1695 | −0.0005 | 0.5445 | −0.2285 | 0.6105 |
| CHAF1B | 0.603 | 0.354 | 0.263 | 0.189 | 0.027 | −0.155 | 0.879 | 0.008 | 0.139 | 0.219 | 0.391 | −0.179 | −0.281 | 0.14 | 0.558 |
| CHC1 | −0.273 | −0.142 | −0.589 | −0.361 | −0.702 | −0.522 | −0.134 | −0.374 | −0.503 | −0.344 | −0.4 | −0.291 | −0.82 | −0.825 | −0.085 |
| CHCHD2 | −0.09 | −0.702 | 0.7 | 0.329 | 0.361 | −0.75 | 1.587 | 0.106 | −0.104 | −1.22 | 2.679 | 4.198 | 3.578 | 1.671 | 2.403 |
| CHCH05 | −1.079 | −1.992 | −1.296 | 2.005 | −1.188 | −1.267 | −0.212 | −0.209 | 0.109 | 0.86 | 0.814 | 2.243 | 2.173 | 3.285 | 2.419 |
| CHI3L1 | 1.987 | −0.66 | 0.395 | −0.288 | −0.453 | 0.322 | −0.087 | 0.433 | 1.169 | −0.589 | 0.583 | 0.222 | 0.039 | 0.318 | −0.787 |
| CHI3L2 | 0.061 | −0.092 | −0.199 | −0.29 | −0.134 | 0.264 | 0.654 | 0.139 | 0.248 | −0.093 | 1.22 | 2.056 | 1.376 | 0.94 | 1.008 |
| CHKA | 0.115 | −0.271 | −0.14 | −0.438 | −0.315 | −0.199 | −0.522 | 0.403 | 0.682 | 0.38 | −0.28 | 0.63 | 0.132 | 0.73 | 3.484 |
| CHCDL | 0.0045 | −0.1325 | 0.0185 | −0.2585 | −0.1465 | 0.0665 | 0.2075 | 0.2735 | 0.1515 | 0.0365 | −0.0835 | 0.1235 | 0.2395 | −0.1805 | −0.3215 |
| CHRDL2 | −0.724 | 0.244 | −0.589 | 0.412 | −0.445 | −0.285 | 0.156 | 0.117 | −0.313 | −0.827 | −1.229 | −0.401 | −0.911 | −1.005 | −0.602 |
| CHST2 | −0.4125 | −0.0485 | −0.2895 | −0.6975 | −0.0865 | −0.1795 | −0.0275 | 0.0755 | −0.5215 | 0.0195 | −0.3395 | 0.0635 | −0.3095 | −0.7505 | 0.0025 |
| CHURC1 | −0.145 | 0.382 | −0.707 | −0.014 | 0.148 | 0.206 | −0.688 | −0.214 | −0.416 | −0.698 | −1.224 | −0.597 | −1.266 | −1.023 | −0.742 |
| CIR | −0.1795 | 0.0225 | 0.4525 | 0.1655 | 0.1035 | 0.0665 | 0.6615 | 0.1895 | −0.3355 | −0.3025 | −0.5075 | 0.1395 | −0.5115 | −0.2555 | −0.2125 |
| CIRBP | −0.8515 | −0.9015 | −0.5565 | 0.0115 | 0.1975 | −0.1385 | −0.0005 | 0.7725 | −0.9755 | −1.0615 | −0.8935 | −0.2905 | −0.7965 | −0.3095 | 0.7525 |
| CKLF | 0.1185 | −0.6575 | −0.7785 | −0.8355 | −0.4605 | 0.1735 | 0.8475 | 1.0575 | −0.2755 | −0.9255 | 0.7335 | 0.8405 | 0.9155 | 1.4415 | 1.1805 |
| CKLFSF6 | 0.5105 | 1.8415 | 1.3965 | 0.9795 | −0.6805 | 0.2205 | 1.2895 | −1.5405 | 0.9965 | 0.8105 | 0.3565 | −1.9255 | 0.1075 | 0.7515 | −1.4615 |
| CKLFSF7 | −0.538 | −0.021 | 0.063 | 0.515 | −0.085 | −0.295 | −0.54 | −0.423 | −0.468 | 0.089 | 0.458 | 0.54 | −0.059 | 0.965 | 0.958 |
| CKMT1 | −0.334 | 0.422 | −0.514 | 0.295 | 0.464 | −0.983 | −0.133 | −0.471 | 0.296 | −0.417 | 0.334 | −0.51 | 0.204 | 1.331 | 1.437 |
| CKS1B | 0.262 | −0.436 | −0.538 | 0.27 | 1.333 | −0.522 | 0.586 | −0.31 | −0.225 | −0.535 | 0.079 | −0.138 | 0.36 | −0.391 | −0.002 |
| CKS2 | −0.0495 | −0.7535 | 0.5895 | −0.3815 | 0.0775 | 0.6875 | 0.2915 | −0.6345 | −0.7185 | −0.9705 | 2.0115 | 0.3585 | −0.3485 | 0.2085 | −1.6315 |
| CL640 | 1.1655 | −0.0645 | −0.6575 | −0.1305 | −0.5825 | 1.4345 | 2.8535 | −1.0945 | 0.3985 | −0.0045 | 0.9015 | 0.9305 | 0.7705 | 0.9795 | −0.9855 |
| CLDN23 | 0.2795 | −0.7205 | −0.3805 | 1.5375 | −0.6755 | 0.3125 | 2.3165 | −0.6275 | −0.0905 | 0.2095 | 0.4305 | 0.7675 | 0.4395 | 0.1255 | −0.8045 |
| CLDN3 | 1.3235 | −0.4385 | 0.5165 | 0.9695 | −0.4835 | −0.5515 | −0.4035 | 0.8355 | 0.2405 | −0.9345 | −0.7465 | 0.1855 | 0.4445 | −0.6035 | 1.8085 |
| CLDN4 | −0.523 | −1.598 | 0.395 | 0.289 | −0.687 | −0.788 | −0.939 | −2.085 | −0.907 | 0.044 | −0.148 | −1.612 | −1.596 | −0.443 | −1.342 |
| CLDN8 | −0.5765 | 0.1275 | −0.8085 | −0.5585 | −0.6705 | −0.5946 | 1.0435 | −0.2545 | 0.5795 | 0.8505 | −0.8405 | −0.9285 | −1.3625 | 0.8775 | −0.2355 |
| CLGN | −0.2365 | 0.3275 | 0.0635 | −0.1945 | 0.6445 | −0.1515 | −0.3065 | 0.2625 | 0.1395 | −0.5265 | 0.6555 | −0.0345 | 0.0005 | 0.7535 | 0.6005 |
| CLIC6 | −0.202 | 0.385 | 0.88 | 2.441 | 1.254 | −0.251 | 0.825 | −0.193 | −0.78 | 0.29 | −0.841 | −0.742 | −0.909 | −0.806 | −0.119 |
| CLNS1A | 2.3585 | −1.2525 | −0.0465 | −0.9355 | 0.1485 | 0.0035 | −0.0625 | −0.0235 | −1.8495 | 0.2315 | −0.8355 | −1.4935 | 0.5485 | 0.3515 | −0.8895 |
| CLTC | −0.615 | −0.259 | −0.799 | 0.211 | −0.075 | −0.162 | 0.302 | −0.194 | 0.163 | −0.245 | −0.036 | 1.021 | 2.962 | 1.293 | 1.163 |
| CLU | −1.403 | 0.144 | 0.581 | 0.586 | 0.252 | 0.223 | −0.105 | −0.289 | 0.21 | −0.077 | 0.113 | 0.603 | 0.21 | 0.37 | 0.95 |
| CMAS | 0.1165 | 1.6485 | 0.5895 | 1.1225 | 0.2995 | −0.3065 | 0.4805 | −0.9145 | −0.2665 | −0.8165 | −1.0335 | −0.9555 | −1.0925 | −1.1365 | −0.2455 |
| CML65 | −0.238 | 2.029 | −0.209 | 0.303 | −0.285 | −0.358 | −0.091 | 0.931 | −0.128 | −0.854 | 0.404 | 0.001 | −0.736 | 0.718 | 1.034 |
| CNIH | 0.035 | −0.078 | 0.159 | −0.195 | 0.227 | −0.148 | −0.081 | 0.362 | 0.273 | −0.42 | 0.077 | −0.195 | 2.641 | −0.277 | −0.232 |
| CNOT10 | −0.9385 | −0.2485 | −0.0485 | −0.5035 | 0.9695 | −0.0465 | −0.3445 | 1.0015 | −0.5555 | −0.7105 | −0.3445 | −0.4015 | −0.2405 | 0.7855 | −0.0865 |
| CNOT2 | 0.49 | −0.378 | 0.402 | −0.126 | −0.391 | −0.12 | −0.698 | 0.068 | −0.086 | −0.338 | −0.158 | −0.02 | 0.025 | −0.339 | −0.169 |
| CNOT7 | 3.324 | 3.257 | 1.851 | 1.343 | 1.934 | 1.583 | 3.178 | 2.903 | 2.864 | 1.704 | −1.405 | −2.209 | −1.917 | 1.734 | −1.555 |
| CNP | 0.0745 | −0.8765 | −0.5975 | −0.7295 | −0.2135 | −0.6465 | 0.2285 | −0.0015 | 0.1135 | −0.0555 | 0.0425 | 0.0585 | −0.4565 | −0.7025 | 0.0575 |
| CNTNAP2 | 0.102 | 0.35 | 0.178 | −0.08 | 0.074 | 0.045 | −0.284 | −0.175 | 0.174 | 1.89 | 0.313 | 0.101 | 0.075 | 0.191 | −0.013 |
| COASY | −0.6865 | −0.7045 | 0.0005 | −1.1735 | 0.1625 | −1.2015 | −0.2505 | 0.4895 | 0.4595 | −0.4135 | 1.3315 | 1.4065 | 2.3475 | 0.3325 | 0.3795 |
| COL11A2 | −1.712 | −0.427 | 0.027 | −1.052 | 1.577 | −1.026 | −0.982 | −0.291 | −0.002 | −1.253 | −0.584 | −0.457 | −1.064 | 0.482 | −1.229 |
| COL27A1 | −0.517 | 0.363 | 0.363 | 0.122 | 0.253 | −0.504 | −0.392 | −0.266 | 0.422 | −0.525 | 0.35 | 0.312 | 0.056 | −0.39 | −0.204 |
| COL4A5 | 0.3815 | −0.4055 | 0.6265 | 0.0655 | −1.5615 | 0.4355 | −1.1625 | −0.7525 | −0.1555 | −1.0435 | 1.0545 | 1.3775 | 0.5335 | 1.4965 | 0.9915 |
| COL8A1 | −0.655 | 0.029 | −0.159 | 0.2 | 1.377 | 0.192 | −0.779 | −0.341 | −0.368 | −0.191 | −1.174 | −0.769 | −1.252 | −1.256 | −0.789 |
| COL8A3 | 0.25 | −0.638 | 0.039 | 0.44 | −0.526 | 0.166 | 0.085 | 0.61 | −0.023 | 0.638 | −0.038 | 0.845 | 0.671 | 0.117 | 0.298 |
| COMMD1 [symbol] | 0.0055 | −0.0095 | −0.0635 | −0.2495 | −0.0935 | 0.2965 | −0.3025 | −0.0385 | 0.4575 | 0.1505 | 0.1465 | 0.0635 | 0.2215 | 0.3485 | 0.0495 |
| COMMD5 | −0.1655 | 0.1405 | 0.4045 | −0.1645 | 0.2855 | −0.1485 | 0.1575 | 0.1785 | 1.6875 | 0.1825 | −0.1915 | −0.2125 | −0.0965 | −0.3305 | −0.6005 |
| COPS7B | −0.026 | 0.19 | 0.179 | 0.082 | 0.431 | 0.774 | −0.103 | −0.231 | 0.285 | 0.058 | 0.106 | −0.315 | 0.108 | −0.327 | 1.539 |
| CORO6 | 0.4555 | −0.4525 | −0.2605 | −0.0275 | −0.2995 | −0.5265 | 0.2155 | −0.4165 | −0.6695 | 0.1085 | 0.0255 | 0.0595 | −0.1635 | 0.0575 | −0.4315 |
| CORT | −0.214 | 0.997 | 0.025 | 0.416 | 0.21 | 0.04 | 0.449 | −0.358 | −0.201 | −0.239 | −0.426 | −0.262 | −0.619 | 0.008 | 1.178 |
| COX17 | −0.237 | 1.104 | 0.068 | 0.468 | 0.244 | −0.006 | 0.488 | −0.38 | −0.198 | −0.139 | −0.483 | −0.227 | −0.4 | −0.047 | 1.09 |
| COX5A | −0.341 | −0.253 | −0.509 | 0.18 | −0.184 | −0.657 | −0.123 | −0.569 | −0.105 | −0.46 | −0.392 | −0.395 | −0.959 | −0.548 | 0.228 |
| COX6A | −0.7425 | −0.1915 | −0.7325 | −0.4395 | −0.7085 | −1.2855 | −1.1295 | 0.8365 | −0.5355 | −0.7655 | −1.2795 | 0.0045 | −0.0045 | −1.2715 | −0.4755 |
| COX8A1 | 0.7445 | 0.3715 | 0.3205 | −0.1635 | 0.2395 | −0.3425 | −0.1035 | −0.5715 | −0.3785 | 0.1445 | −0.2785 | 0.0095 | −1.2685 | −0.2675 | 0.4335 |
| COX8C | −0.0435 | −0.8745 | 4.9935 | 3.9155 | 1.7165 | 0.6965 | 5.3795 | 1.7255 | 4.8815 | 4.0795 | 3.4905 | 1.3745 | 5.0655 | 0.9805 | 2.4885 |
| COX7A2 | 0.578 | 1.161 | 0.93 | 0.234 | −0.898 | −0.307 | 0.229 | 0.339 | 0.691 | −0.646 | 0.391 | −0.405 | 0.469 | 0.514 | 0.31 |
| CP | −0.6175 | −0.6375 | −0.4685 | −0.7945 | −0.2005 | −0.3445 | −0.4895 | −0.3295 | 0.1295 | −0.7175 | −0.4875 | −0.2535 | −0.5145 | −0.3985 | −1.0505 |
| CPAMD0 | −0.414 | −0.572 | −0.685 | −0.724 | −0.136 | 0.169 | −0.539 | −0.069 | −0.15 | −0.423 | −0.035 | −0.034 | −0.802 | −0.831 | −1.22 |
| CPB1 | 0.2405 | 0.0145 | 0.2525 | 0.3005 | −0.4375 | 0.4655 | 0.0615 | −0.5435 | 0.0435 | −0.2985 | −0.7175 | −1.1755 | −0.5925 | −2.2235 | −1.9745 |
| CPEB3 | 0.453 | 0.384 | −0.318 | 0.422 | 0.812 | −0.138 | 0.384 | −0.219 | −0.323 | −0.065 | 0.326 | 0.543 | 0.138 | 0.926 | 0.346 |
| CPEB4 | −0.752 | −0.617 | −0.434 | −0.763 | −0.534 | −0.32 | 1.269 | −0.781 | −0.033 | −0.218 | −0.018 | 2.417 | 1.634 | 1.337 | −0.112 |
| CPSF3 | 0.0595 | 1.2405 | 0.6475 | −0.2195 | 0.4865 | 0.9685 | −1.2435 | 0.0825 | 0.6355 | 0.9275 | −1.2905 | −1.5895 | −0.5325 | −1.2765 | −2.4455 |
| CRABP1 | −1.706 | 0.98 | 0.121 | −2.329 | 0.236 | −1.788 | 0.242 | −0.84 | 0.452 | −0.417 | −0.706 | −0.997 | −1.462 | −3.239 | −2.566 |
| CREB3L4 | 0.931 | 0.796 | 0.383 | −0.429 | −0.056 | −0.503 | 1.021 | −0.262 | 0.151 | −0.131 | 0.881 | 0.02 | 1.642 | −0.138 | −0.349 |
| CRIP1 | −0.202 | −0.148 | −0.667 | 0.786 | −0.516 | 0.351 | 0.668 | 0.03 | 0.384 | −0.574 | −1.011 | −0.612 | −0.462 | −1.215 | −0.914 |
| CRYBB2 | −1.033 | −0.207 | −0.429 | 0.95 | −0.216 | −0.207 | −1.623 | −1.055 | 0.985 | −0.462 | −0.364 | −0.023 | −0.414 | −0.491 | −0.652 |
| CRYL1 | 0.875 | 0.987 | −0.288 | 0.15 | −0.443 | 0.042 | 0.931 | −0.113 | 0.374 | 1.302 | 1.058 | 1.494 | 1.495 | 1.644 | 1.216 |
| CRYZ | 0.1065 | 0.7275 | −0.0035 | 0.1355 | 0.4576 | 0.0805 | −0.2985 | −0.1225 | 0.0285 | −0.2345 | 0.4535 | −0.0345 | −0.3725 | 1.1765 | 0.8435 |
| CSDA | 0.4535 | 0.0585 | −0.2185 | −0.2495 | 0.5535 | 0.6985 | 1.4995 | 0.5625 | −0.0925 | −0.3965 | 0.2905 | 0.3255 | 0.2835 | −0.0585 | 0.3195 |
| CSE1L | −0.7495 | 0.7165 | 0.7435 | −0.4755 | −0.4285 | 0.1775 | −0.0115 | 0.2525 | −0.4455 | 0.0375 | 0.4345 | 2.0495 | −0.8615 | −0.9395 | −0.1405 |
| CSK | −1.0815 | −0.2795 | 1.7555 | 0.3575 | −0.5455 | 0.2355 | −0.0715 | 0.5545 | −0.9625 | −0.3325 | −0.1025 | 0.5555 | −0.3215 | 1.4965 | −0.0385 |
| CSRP1 | 1.3705 | −0.1525 | 1.5395 | 1.2175 | −0.3705 | 0.3285 | 3.5225 | −0.0805 | 0.3335 | 1.0115 | −1.1035 | 1.8375 | −0.4085 | 0.4815 | −0.7365 |
| CSRP2 | 2.248 | −0.432 | 0.725 | 1.837 | 0.696 | 0.238 | 1.504 | −0.511 | 0.222 | 0.885 | 1.21 | 0.948 | 1.313 | 1.311 | 0.886 |
| CST5 | 1.2385 | 0.2935 | 0.8085 | 0.4195 | −0.2675 | 0.4125 | −0.8425 | −0.8185 | −0.6565 | −0.1115 | 0.6935 | 2.3765 | 2.8145 | 0.4495 | 2.4865 |
| CSTB | −1.427 | −0.35 | −0.486 | −0.971 | −0.14 | −0.451 | −0.434 | −0.079 | 0.473 | 0.257 | −0.081 | 0.2 | −0.493 | 0.092 | 0.254 |
| CTAG1B | 0.0605 | 0.6815 | 0.3555 | 0.3525 | 1.3095 | −0.3145 | −0.4555 | −0.4755 | 0.0665 | 0.2425 | 0.0655 | 0.3485 | −0.6485 | −0.7955 | −0.5745 |
| CTBP2 | −0.627 | −0.659 | −0.529 | −0.974 | 1.201 | −0.687 | −1.452 | −0.301 | −0.897 | −0.709 | −1.28 | −0.682 | −1.127 | −2.188 | −1.48 |
| CTNNA1 | −0.545 | −0.374 | 0.132 | 0.068 | −0.115 | 1.034 | −0.205 | −0.107 | 0.218 | −0.238 | −0.829 | 0.458 | 0.885 | −0.796 | 1.069 |
| CTNNAL1 | −0.1655 | 0.8425 | 0.5805 | 0.2615 | −0.4935 | −0.1875 | 0.2235 | 0.1755 | 0.5625 | 0.0005 | 0.9715 | 1.2865 | 0.4365 | 1.2785 | 1.2075 |
| CTNND2 | 1.058 | −0.565 | −0.195 | 1.028 | 0.498 | −0.124 | 1.738 | −0.478 | −0.532 | 0.09 | 0.122 | 1.683 | −0.355 | 1.076 | 2.281 |
| CTPS | 1.304 | −0.354 | −0.347 | 0.316 | 1.403 | 2.288 | 1.985 | 0.7 | −0.801 | −0.219 | −0.427 | −0.141 | 0.001 | −1.324 | −0.809 |
| CTSL2 | 0.1185 | 0.1395 | 0.3635 | 0.7965 | 0.4205 | 0.0455 | 0.8955 | −0.8325 | −0.1515 | 1.2255 | 0.8185 | −0.2775 | −0.3775 | 0.2005 | 0.1575 |
| CTSS | −2.08 | 0.99 | 0.99 | −1 | 1.92 | −1.01 | 2.33 | 1.14 | 0.261 | −2.29 | 0.01 | 1.65 | 1.839 | 1.438 | 1.588 |
| CTTN | −0.5795 | −0.1585 | −0.7105 | 0.0585 | 1.6945 | 2.1175 | 3.0205 | 3.2275 | 0.9995 | −0.5735 | 1.2055 | −0.0445 | 0.6295 | −1.8815 | −0.8155 |
| CX3CL1 | 0.5675 | −0.3105 | −0.3315 | −0.3385 | 0.3295 | 0.0175 | 0.7275 | −0.5925 | −0.5085 | −0.2105 | −0.8965 | −0.3745 | −0.4535 | 0.7245 | −0.7435 |
| CXCL9 | −1.7955 | 0.5045 | 0.6655 | −2.5645 | 1.7575 | −0.9175 | −0.9315 | −0.7585 | 0.5115 | −0.2045 | −1.0575 | −0.0585 | 1.4495 | −0.2635 | 1.0025 |
| CXorf39 | 0.567 | −0.72 | 0.008 | 0.491 | 0.08 | −0.548 | −0.314 | 0.576 | 0.941 | 1.436 | −1.328 | −1.216 | −2.039 | −1.42 | 0.29 |
| CXXC4 | 0.887 | 0.435 | 0.118 | 1.759 | 0.343 | 2.469 | 1.737 | −0.247 | 0.654 | 1.088 | −0.019 | −0.867 | −0.269 | −0.531 | −0.332 |
| CYB5 | 0.2775 | −0.4565 | 0.0785 | 0.3155 | −0.5505 | 0.1635 | −0.5405 | −0.0385 | 0.0565 | 0.3835 | −0.0025 | 0.9155 | 0.9205 | 0.4825 | 0.7905 |
| CYB561 | 0.55 | −0.722 | −0.189 | −0.344 | −1.383 | 0.298 | −0.456 | 1.065 | 0.845 | −0.414 | 0.289 | 0.914 | 1.508 | −0.39 | −0.212 |
| CYC1 | −0.07 | 0.107 | 0.204 | 0.062 | −0.029 | 0.046 | 0.341 | −0.18 | 0.216 | 0.141 | 0.18 | 0.119 | 0.06 | 0.052 | −0.258 |
| CYHR1 | −1.1265 | −0.7625 | 2.9915 | 1.4355 | 2.0665 | −0.5665 | 0.6305 | −0.5095 | −0.0925 | −0.8055 | −1.0945 | −0.7725 | −0.4645 | −0.6245 | −1.0875 |
| CYP2A13 | −0.16 | 0.474 | 0.285 | −0.471 | −0.19 | 0.051 | 0.232 | 0.384 | −0.176 | −0.539 | 0.519 | 1.011 | 0.742 | 0.125 | 1.142 |
| CYP4X1 | −1.9245 | −1.3735 | −1.7525 | −1.7265 | −1.7675 | −2.6395 | −2.5195 | 0.4925 | −1.8735 | −1.5975 | −1.9895 | −2.1285 | −1.8835 | −1.0535 | −0.3805 |
| D15Wsu75 | −0.9895 | −0.8325 | −0.8575 | −1.0705 | −1.0825 | −1.1205 | −0.9605 | 0.5255 | −1.0485 | −0.9045 | −0.8325 | −0.8245 | −1.0215 | −0.5515 | 0.2495 |
| DACH1 | −2.03 | −1.797 | −2.048 | −1.991 | −2.02 | −2.478 | −2.209 | 1.095 | −1.559 | −1.582 | −2.605 | −1.818 | −2.155 | −0.971 | −0.135 |
| DACH1 | −0.211 | 0.609 | −0.07 | 0.118 | −0.183 | 0.393 | 0.115 | 0.281 | 0.362 | 0.269 | 0.518 | 0.184 | 0.829 | 0.212 | 0.028 |
| DACH1 | −0.268 | 0.112 | 0.399 | 0.763 | 0.947 | 1.526 | 0.464 | 0.479 | 0.132 | 0.367 | 0.563 | 0.374 | 0.449 | −0.092 | −0.249 |
| DAP3 | −0.8155 | −0.9195 | −0.2525 | −0.0015 | −0.0035 | 0.1435 | −0.5895 | 0.0345 | −0.3845 | −0.2065 | −1.1035 | −0.5295 | −0.6505 | −1.3425 | −0.7805 |
| DAPP1 | 2.749 | −0.056 | 0.787 | 1.815 | 0.145 | 0.506 | 0.342 | −0.308 | 0.083 | 0.545 | 1.069 | 1.061 | −0.17 | −1.534 | −1.15 |
| DAZAP2 | −0.536 | 0.399 | 0.788 | 0.41 | 0.062 | 1.722 | −0.546 | 0.579 | −0.107 | 0.113 | 0.569 | 0.846 | −0.01 | 0.741 | 0.411 |
| DBI | 0.1055 | 0.8345 | 0.4085 | 0.2785 | 0.7665 | 0.1665 | 0.4285 | −0.2095 | 0.0105 | −0.3225 | 0.6285 | 0.3335 | −0.1435 | 0.5935 | 0.7455 |
| D9N1 | −0.058 | −0.978 | 0.119 | 0.252 | −0.436 | 0.518 | 0.338 | 0.391 | −0.175 | −0.431 | 0.772 | 1.688 | 0.042 | 0.477 | 1.117 |
| DC13 | 0.0885 | 0.3245 | −0.0065 | −0.2876 | 0.6195 | 0.1125 | 0.1065 | 1.1795 | 0.0035 | 0.7995 | 0.3665 | 0.2875 | −0.4235 | 0.5535 | 0.0085 |
| DCPS | −0.0705 | 0.1905 | 0.7476 | 0.0905 | −0.2455 | −0.2575 | −0.4735 | −0.0775 | −0.3985 | −0.3655 | 1.0645 | −0.0435 | −0.4085 | −0.4565 | −0.4065 |
| DDHD2 | 0.8105 | 0.1635 | −0.2985 | 0.6845 | −0.1415 | −0.0135 | 0.3005 | 0.1995 | −0.2695 | 0.8525 | 0.2965 | 0.0065 | 0.4885 | 0.2125 | 2.8425 |
| D0X17 | 0.5305 | 0.2005 | 0.0425 | 0.5465 | 0.5376 | 0.2935 | −0.4105 | −0.0175 | 0.6245 | 0.9395 | −0.2035 | −0.3625 | −0.8755 | −0.1995 | −0.1935 |
| DDX39 | 0.115 | 0.279 | 0.55 | −0.027 | −0.843 | 0.221 | −1.337 | 0.103 | −0.644 | −0.759 | 0.093 | 0.085 | 0.006 | 0.824 | 0.308 |
| DDX48 | 0.7965 | −0.6465 | −0.5775 | −0.8545 | −0.0355 | 0.5955 | 0.5865 | 1.9405 | 0.3945 | 0.3185 | 1.0915 | 1.1435 | 0.5205 | −1.2085 | 0.1005 |
| DDX58 | −0.5495 | −0.1695 | 0.0715 | −0.0155 | 0.8765 | 0.0155 | −0.6605 | −0.1305 | −0.4435 | 0.1185 | 1.0005 | 0.3815 | −0.2505 | −0.0485 | 0.5465 |
| DEF6 | 2.921 | 0.39 | 0.848 | 0.673 | 0.585 | 0.693 | 2.08 | −0.277 | 0.502 | −0.43 | 0.781 | 1.798 | 0.566 | 1.583 | 0.321 |
| DEK | −0.714 | −0.221 | −0.533 | 0.811 | 0.173 | 0.379 | 0.366 | −0.657 | −1.021 | −0.353 | −0.804 | −0.85 | −1.269 | −1.204 | −0.243 |
| DHCR7 | 0.854 | 1.202 | 0.946 | 2.077 | −0.459 | 0.452 | 1.147 | 0.33 | 0.101 | 0.215 | 2.027 | 0.804 | 0.647 | 1.529 | −0.145 |
| DHRS7 | −0.0295 | 0.1215 | 0.4575 | 0.2815 | 0.1335 | −1.1255 | 0.1595 | 0.7325 | 0.5835 | −1.0665 | 0.8935 | −0.2595 | −1.1085 | 0.1705 | 1.0205 |
| DHX57 | 3.4675 | 1.0235 | 0.5875 | 1.1405 | −1.3415 | 3.1205 | −0.3165 | 0.9485 | 1.0285 | 0.1055 | −1.0385 | −1.2095 | −0.7885 | −1.4805 | −2.0885 |
| DIRC1 | 0.134 | 0.462 | 0.572 | −0.264 | 0.206 | −0.351 | 0.382 | −0.072 | 0.284 | 0.18 | 0.82 | −0.379 | −0.137 | 0.532 | 0.962 |
| dJ222E13 | −0.279 | −0.48 | 0.795 | −0.057 | −0.259 | 0.095 | 0.614 | 0.759 | −0.37 | −0.12 | 0.199 | 1.055 | 2.277 | 2.418 | 2.325 |
| DKFZP434 | 0.781 | 0.876 | 0.819 | 0.33 | −0.152 | 1.319 | 1.88 | −0.068 | −0.246 | 0.808 | 0.447 | 1.488 | 0.037 | 0.314 | 1.469 |
| DKFZp434 | 0.4885 | 0.3855 | 0.2305 | 0.2685 | 0.1885 | −0.3075 | 0.0265 | 0.6685 | 0.0915 | 0.7115 | 0.7805 | 0.8549 | 0.2115 | 1.5875 | 1.2805 |
| DKFZP434 | −0.328 | −0.578 | −0.108 | −0.02 | −0.5 | 0.324 | 0.085 | −0.339 | −0.003 | −0.806 | −0.43 | −0.32 | −0.228 | −0.391 | −1.243 |
| DKFZp434 | 0.1755 | −0.4415 | −0.0315 | 0.0835 | 0.0025 | 0.1405 | −0.3585 | 0.2545 | 0.2485 | −0.2555 | −0.7715 | −0.5985 | −0.3005 | −0.8855 | −0.9155 |
| DKFZP564 | −0.4835 | −0.2215 | −0.0835 | −0.5225 | −0.7045 | −0.0915 | −0.1925 | −0.0725 | 0.9605 | 0.3995 | −0.0665 | 0.6605 | −0.2445 | 0.1445 | 0.2825 |
| DKFZp564 | −1.37 | 0.688 | −0.007 | 0.469 | −0.105 | −0.098 | −0.327 | −0.103 | 0.26 | −0.451 | 0.017 | 1.121 | 0.824 | 0.142 | −0.642 |
| DKFZP564 | −1.3195 | −0.6705 | −0.0575 | 1.2495 | 0.3085 | 0.1935 | −0.1735 | −0.0025 | −1.1545 | −0.4945 | −0.9045 | −0.5675 | −0.7055 | −0.7945 | 0.9055 |
| DKFZP564 | −0.124 | 0.654 | 0.468 | 0.486 | 1.209 | 0.258 | −0.771 | −0.466 | −0.149 | −0.335 | 0.035 | −0.392 | −0.539 | −0.208 | 0.044 |
| DKFZP564 | −0.204 | −0.012 | −0.278 | −0.936 | 0.44 | 0.48 | 0.038 | 0.148 | −0.158 | −0.494 | 0.458 | −0.909 | 0.092 | 0.51 | −0.184 |
| DKFZp586 | 0.5995 | −0.3075 | 0.5085 | 0.1955 | −0.4055 | −0.2285 | −2.1515 | −0.2125 | 5.5645 | 0.2445 | 0.2585 | −0.9105 | 1.0145 | −0.2015 | 0.3765 |
| DKFZp761 | 0.05 | 0.049 | 0.024 | −0.142 | −0.087 | −0.192 | 0.043 | 0.143 | 0.021 | 0.195 | 0.302 | 0.306 | 0.819 | −0.045 | −0.027 |
| DLK1 | 0.335 | 0.59 | 0.785 | 0.069 | 0.626 | 0.078 | −0.37 | 0.629 | −0.002 | −0.618 | −1.074 | −1.23 | 0.002 | −1.723 | −0.197 |
| DLX5 | −0.1625 | 0.2465 | −0.3985 | −0.8785 | 0.3555 | −0.1085 | 1.3975 | −0.3855 | 0.2975 | 0.0315 | −0.5405 | 0.2145 | −0.0675 | −0.1615 | 0.3575 |
| DNAJA4 | −2.3685 | −2.2005 | −1.1945 | −2.2525 | −1.8005 | −3.0405 | −2.7835 | −2.2175 | −1.9785 | −1.6455 | −2.1615 | −1.1935 | −2.8855 | −0.0165 | −1.7045 |
| DNAJC1 | −0.291 | 1.3 | −0.894 | 1.639 | 0.758 | 0.171 | 1.285 | −0.002 | −0.233 | −0.204 | 0.06 | −1.424 | 0.227 | −0.194 | 0.928 |
| DNAJC12 | −2.929 | −2.406 | −0.086 | −2.584 | −0.3 | −2.576 | −1.247 | −0.182 | 0.04 | 0.717 | −2.928 | 0.417 | −2.879 | 1.399 | −1.481 |
| DNAJD1 | 0.885 | −0.014 | −0.081 | 0.162 | 0.231 | −0.171 | 0.04 | 0.468 | 0.595 | 0.187 | 0.402 | −0.127 | −0.125 | −0.078 | 1.913 |
| DNALI1 | −0.232 | 1.074 | 0.754 | −0.305 | 0.816 | 0.646 | −0.593 | −0.056 | 0.223 | 2.147 | 0.122 | 1.317 | −0.424 | −0.145 | 0.235 |
| DNMT1 | −0.5325 | −0.7275 | 0.4065 | 0.1755 | −0.2415 | 0.4855 | 0.5825 | 0.8965 | 0.2115 | −0.5815 | −0.0495 | 0.9455 | 0.6065 | −1.1025 | −0.0715 |
| DOX1 | −0.0855 | −0.0735 | −0.3425 | 0.1055 | −0.0455 | 0.3345 | −0.0005 | 0.4605 | 0.0985 | 0.3845 | 0.0285 | −0.3955 | −0.1805 | −0.0815 | 0.2715 |
| DSCR1 | −2.8665 | −0.1525 | −0.0235 | 1.3595 | −0.1585 | −0.5565 | −0.1815 | 0.3955 | 0.0235 | 0.4005 | 1.1885 | 2.5665 | −0.2535 | 1.5845 | 2.7575 |
| DSCR1L2 | 0.1885 | 0.1165 | 0.0675 | 0.3255 | −0.0885 | −0.0715 | −0.3025 | 0.0215 | −0.1445 | 0.5965 | 0.3155 | 0.3215 | −0.3635 | 0.5935 | 0.6605 |
| DSP | −0.1525 | 0.0335 | −0.0395 | −0.1135 | −1.5015 | −0.6085 | 0.5935 | 0.1945 | −0.0225 | 0.7725 | −0.0825 | 0.8135 | 0.4935 | −0.3795 | 0.6345 |
| DTYMK | −0.1595 | 0.6635 | 0.1725 | −0.4185 | 0.6445 | 1.1905 | 0.0025 | 0.4955 | −0.2105 | −0.7945 | −0.3185 | −0.4755 | −0.3985 | 0.0625 | −0.2835 |
| DUSP23 | 2.6585 | 0.5335 | 1.2485 | −0.0415 | −0.3425 | 0.5655 | 0.3835 | 0.1065 | −0.7795 | −1.8865 | −1.7595 | 0.5385 | −0.6335 | 0.2595 | −1.3285 |
| DUSP4 | −0.0825 | −0.7375 | −0.4095 | −0.5075 | −0.8355 | −0.1985 | −0.0835 | −0.0775 | 0.5475 | 0.2415 | −0.0875 | 0.6875 | 0.9845 | −0.0885 | 0.0365 |
| DUSP5 | 0.168 | 0.098 | 0.04 | −0.064 | 0.89 | 0.051 | 0.439 | −0.082 | 0.237 | −0.156 | −0.017 | −0.037 | −0.047 | 0.117 | −0.009 |
| DVL1 | −0.771 | −0.246 | −0.743 | −1.172 | −0.577 | 0.372 | 0.183 | 0.178 | 0.36 | 1.111 | −0.154 | 2.04 | −0.7 | 0.308 | 1.064 |
| DXYS155 [symbol] | −0.184 | 0.192 | 0.859 | 0.388 | 0.488 | −0.018 | −0.398 | 0.025 | 0.021 | −0.109 | 0.513 | 0.497 | 0.595 | 0.223 | 0.772 |
| DYRK4 | 1.1815 | −0.6705 | −0.4835 | −0.1665 | 0.8575 | 1.6535 | 0.3315 | 0.2775 | 0.3935 | −0.4155 | −0.4425 | −0.2355 | −0.3355 | −0.5785 | −0.4405 |
| o(y)2 | 2.868 | 1.46 | −0.353 | 0.698 | 0.258 | 0.099 | −0.011 | 0.189 | 0.053 | 0.039 | −0.224 | 0.207 | −0.234 | −0.218 | 0.225 |
| EAF2 | −1.5865 | −1.7795 | −0.0085 | 0.9575 | −0.7135 | −0.2615 | −0.7965 | −0.2835 | −1.0716 | 0.1895 | −0.7565 | −0.7195 | −1.2415 | −0.1255 | 1.9245 |
| EAP30 | 0.0425 | −0.9325 | −0.5725 | 0.4025 | −1.7735 | −0.7045 | 1.0085 | 0.1785 | 0.7845 | 1.6535 | −0.4825 | 0.4985 | −0.8965 | 0.2925 | 2.8675 |
| ECHDC1 | −0.6275 | −0.5065 | −0.4585 | 0.7535 | 0.8385 | 0.8955 | −0.8785 | −0.2485 | −0.0295 | −0.1645 | −0.4885 | −0.8005 | −0.8605 | −0.5535 | −1.3715 |
| ECHDC3 | −0.2255 | −0.5065 | −0.7685 | −1.2055 | −0.1115 | −0.8285 | 1.3245 | −0.0615 | −0.0265 | 0.1225 | −1.1885 | 0.0135 | −0.9525 | −0.9375 | −0.6235 |
| EEF1A2 | 0.125 | 0.034 | 0.485 | 0.201 | −0.123 | 0.312 | 0.706 | −0.103 | 0.128 | −0.038 | 0.549 | 0.509 | 0.878 | 1.096 | 0.516 |
| EFHC1 | −1.1775 | −1.7375 | 0.1535 | 0.4495 | 0.1116 | −0.6995 | 0.4155 | −0.9485 | −0.2025 | 0.2035 | 0.3135 | −0.0005 | −0.1955 | 0.0255 | 0.2135 |
| EFNA4 | −0.2915 | −0.3945 | 0.2245 | 0.0225 | −0.2916 | 0.4075 | 0.0555 | 0.4335 | 0.3535 | 0.4155 | −0.3185 | 1.0285 | 0.9695 | 1.0285 | 0.6905 |
| EFS | −0.1175 | −0.6355 | −0.3625 | −0.2905 | −0.0805 | 0.0255 | −0.0545 | −0.2916 | −0.0995 | −0.1435 | −0.1935 | 0.5805 | −0.0765 | 0.0325 | 0.0215 |
| EIF2C2 | 0.5125 | 0.4115 | 0.7815 | 0.6045 | −0.2245 | 0.2505 | 0.3305 | 1.0995 | 0.0635 | 1.1465 | 1.1735 | 0.0825 | 0.2035 | 1.3205 | 0.3845 |
| EIF2C4 | 0.533 | −0.497 | 0.313 | 0.189 | −0.141 | 0.292 | 0.642 | 0.718 | −0.099 | 0.142 | 0.512 | 0.461 | 0.898 | 1.002 | −0.153 |
| EIF4EBP1 | −0.0395 | 0.0675 | 0.5495 | −0.1275 | 0.1225 | −0.0665 | 0.2565 | 0.4285 | 0.0975 | −0.7935 | −0.4225 | 0.0865 | 0.2585 | −0.3825 | −0.1725 |
| EIF4G1 | −0.643 | −1.171 | −0.081 | −0.149 | −0.842 | 0.039 | 0.408 | 0.163 | −0.555 | −0.626 | 0.164 | 0.892 | −0.208 | −0.554 | 1.61 |
| EIF4G3 | 0.962 | 0.439 | 0.984 | −0.029 | 1.7 | 0.712 | 0.499 | 0.112 | −0.811 | 0.113 | −0.808 | −0.738 | −0.873 | −1.474 | −0.492 |
| ELAC1 | −0.359 | 1.294 | 0.677 | −0.837 | 0.424 | 1.017 | −0.457 | 0.285 | 0.273 | 0.699 | −0.631 | 0.37 | 0.402 | −1.04 | −0.288 |
| ELL2 | 0.9695 | −0.7485 | 0.0215 | −0.4595 | −1.0995 | −0.3365 | 1.0275 | 0.1685 | 1.1805 | −0.8475 | −1.2855 | −0.9465 | −1.5865 | −1.9215 | −1.7635 |
| ELL3 | −0.0585 | −0.3235 | 0.0685 | 0.8345 | 0.0115 | 0.1635 | 0.3935 | −0.0995 | −0.3735 | −0.1965 | 4.4775 | 3.2295 | 3.1446 | −0.4485 | −0.1135 |
| ELOVL5 | −1.4985 | 1.3025 | 0.8985 | 0.4055 | 0.9335 | −0.0465 | 0.1585 | 0.3765 | −0.6145 | 1.2525 | −1.1025 | −1.0695 | 0.3765 | −0.7855 | −0.8525 |
| EN1 | −0.0005 | −2.0265 | −0.6405 | −0.7385 | 0.7505 | −0.8695 | −0.9985 | −0.0475 | −0.8775 | −0.9885 | −0.4895 | 0.0365 | −0.2795 | −0.1605 | −1.5025 |
| ENAH | −0.4055 | −0.5905 | −0.0175 | −0.4735 | 0.0495 | 0.4185 | −0.3725 | 0.8135 | −0.2555 | −0.1815 | −1.8365 | −0.3735 | −1.5245 | −1.8475 | −1.8715 |
| EPHX2 | −1.278 | 0.968 | 0.1 | 0.012 | 0.039 | 1.565 | 0.725 | 0.594 | −1.185 | −1.018 | −1.544 | −0.475 | −1.058 | −0.417 | 0.196 |
| EPLIN | −0.5665 | −0.3385 | −0.6665 | −0.1535 | 1.9785 | −0.3345 | 1.2165 | −0.8035 | −0.4075 | 0.6655 | −0.9465 | −0.5595 | −0.7625 | −0.9805 | −0.3925 |
| EPN3 | 3.8325 | 3.2715 | 2.9985 | 1.6495 | 3.3615 | 3.8275 | 3.8815 | 0.0895 | −0.6745 | 2.5115 | 0.3585 | 1.4835 | −0.3285 | −0.5695 | −1.2005 |
| EPOR | 1.248 | −0.451 | 0.645 | 0.663 | 0.663 | 0.815 | 0.988 | −0.236 | 0.373 | 1.722 | 0.547 | −0.054 | 0.621 | 1.215 | 0.347 |
| ERBB2 | −3.9865 | −4.0815 | −4.3555 | −4.1285 | −2.6465 | −3.5125 | −4.0715 | 1.4085 | 0.0535 | −1.4095 | −4.4805 | −2.9085 | −2.5985 | −3.0415 | −2.0275 |
| ERP70 | 1.313 | 0.625 | 1.274 | 1.634 | 1.217 | 1.243 | 0.943 | −0.391 | −0.035 | 0.913 | −0.602 | 0.063 | −0.01 | −0.221 | 0.718 |
| ESR1 | 0.3685 | −0.3305 | −0.0755 | −0.1695 | −0.3675 | −0.1855 | 0.9155 | 0.7595 | 0.3895 | 0.2405 | 0.6125 | 1.7435 | 0.9445 | 1.1485 | 1.4735 |
| ETFA | 4.4715 | 0.0405 | 0.2335 | 0.9255 | −0.6775 | 0.8265 | 0.9735 | 1.2585 | 0.6255 | 1.0215 | 0.6615 | 0.2905 | 0.5565 | −0.1105 | 0.8825 |
| ETV8 | 0.2965 | 1.2315 | 0.3835 | 0.3115 | 0.9415 | −0.0535 | 0.0905 | −0.1305 | −0.3135 | 0.0005 | 0.7355 | 0.8345 | 0.0155 | 1.3365 | 0.7865 |
| EVER1 | 0.0575 | 0.2905 | 0.2195 | 0.2455 | 0.5475 | −0.1405 | −0.3185 | 0.2145 | −0.6875 | −0.2125 | −0.0785 | 0.3705 | −0.7875 | −0.3975 | 0.4475 |
| EXO1 | 0.1315 | −0.0845 | 0.4115 | −0.3495 | 0.0875 | 0.3805 | 0.3405 | 0.1865 | −0.6825 | −0.0705 | −0.4055 | −0.3025 | −0.1765 | 0.5495 | 0.4485 |
| EXOSC9 | 0.107 | 0.436 | −0.275 | 0.701 | 0.171 | 0.387 | 0.004 | −0.229 | 0.462 | −0.003 | 0.545 | −0.208 | −0.291 | 1.659 | 0.389 |
| EXT2 | −0.592 | 0.935 | −0.288 | 0.004 | 0.15 | −0.098 | 0.014 | −0.316 | −0.662 | −1.212 | −0.999 | 0.078 | −0.725 | −0.359 | −1.431 |
| EZH2 | 0.4325 | 0.4195 | 0.3025 | 0.3705 | 0.2826 | −0.3785 | −0.3085 | −0.2585 | 0.1895 | 0.0735 | 0.2665 | −0.0395 | 0.3525 | −0.0465 | −0.2025 |
| FI1R | 0.624 | 2.199 | 1.869 | 0.675 | 1.85 | 1.529 | 2.374 | −0.179 | 0.231 | 1.004 | −0.023 | −0.615 | −1.305 | 1.084 | −0.795 |
| F9 | 6.69 | −0.224 | 0.014 | 0.683 | −1.17 | −0.234 | −1.293 | −1.048 | −0.428 | −0.033 | 0.671 | 1.571 | −0.141 | −0.311 | 1.039 |
| FA2H | −0.364 | −0.183 | 0.322 | −0.213 | −0.035 | 0.115 | 0.475 | 0.124 | 0.302 | 1.72 | −0.697 | −0.007 | −0.362 | 0.467 | 0.025 |
| FABP7 | −0.764 | −0.205 | −0.287 | 0.203 | −0.315 | −0.054 | −0.369 | −0.825 | 0.32 | 0.478 | −1.142 | 0.183 | −0.883 | 0.063 | 0.063 |
| FADD | 0.787 | 0.23 | 0.342 | 1.039 | −0.396 | 0.614 | −0.197 | −0.244 | −0.389 | 0.074 | 1.32 | 0.393 | 0.277 | −0.551 | 0.629 |
| FAHD1 | −0.719 | 0.307 | 0.297 | −0.227 | −0.593 | −0.788 | −0.803 | −0.033 | 0.075 | 0.471 | 0.705 | 1.339 | 1.006 | −0.058 | 0.297 |
| FAHD2A | 1.162 | 0.062 | 0.095 | 0.078 | 0.447 | −0.049 | 0.421 | 0.067 | 0.171 | −0.292 | 0.073 | 0.495 | 0.218 | −0.11 | −0.053 |
| FAM38A | 2.1155 | 0.0055 | −0.5955 | −0.8005 | 3.3235 | 3.8525 | −0.5415 | 0.1295 | 0.6375 | −0.0165 | 0.0785 | −0.3915 | −0.6115 | −0.1715 | −1.0745 |
| FAM3A | −0.1095 | 0.0095 | 0.7075 | 1.6825 | 0.1725 | 1.4815 | 1.4795 | −0.0785 | −0.3455 | −0.1685 | −0.3425 | 0.3915 | 0.1275 | −0.3095 | 0.4055 |
| FAM3B | 0.118 | −0.286 | −0.409 | −0.708 | 1.24 | 1.743 | 1.656 | 0.133 | −0.415 | −0.162 | 0.147 | −0.876 | 0.276 | −1.171 | −1.223 |
| FAM3C | −0.159 | −0.981 | 0.695 | 1.374 | 0.346 | 0.787 | −0.318 | 0.058 | −0.005 | −0.212 | −0.774 | −0.211 | 0.243 | −0.313 | −0.206 |
| FAM46C | 0.794 | 0.385 | 0.182 | 1.905 | 0.246 | 0.144 | 0.004 | −0.164 | −0.238 | 0.644 | 0.695 | 0.155 | −0.729 | 1.379 | 3.002 |
| FAM49B | 0.579 | 0.455 | −0.104 | −0.631 | 0.421 | 0.159 | 1.879 | 1.148 | 0.577 | 0.124 | 2.22 | 1.373 | 0.477 | 1.767 | 1.187 |
| FAM54A | 0.152 | 0.339 | −0.137 | −0.583 | −0.405 | 0.411 | −0.789 | 0.104 | 0.079 | 0.048 | 1.56 | 1.262 | −0.028 | −0.143 | 1.513 |
| FANCA | −0.3285 | 0.0015 | −0.2155 | −0.6505 | −0.5995 | −0.4925 | −0.5325 | 0.2255 | 1.0085 | −0.8525 | 1.0875 | 1.0835 | −0.4555 | 2.1975 | 0.1315 |
| FANCE | −0.0245 | −2.3515 | −1.5255 | −1.5845 | 0.7395 | −0.0875 | 0.3105 | 0.0215 | −0.3955 | −0.8575 | −2.4715 | −0.2465 | −1.1685 | −2.9815 | −2.8425 |
| FBL | 0.038 | −0.366 | −0.024 | −0.268 | 0.298 | 0.173 | −0.233 | 0.279 | 0.686 | −0.013 | 0.498 | 0.031 | −0.19 | −0.629 | 0.324 |
| FBP1 | 0.194 | 0.683 | 1.556 | 1.628 | 1.847 | 0.742 | 1.075 | −0.009 | −0.081 | 1.14 | 0.337 | 0.305 | 0.258 | 0.403 | −0.382 |
| FBS1 | 0.3915 | −0.1715 | −0.5865 | −0.8965 | 0.0165 | −0.5005 | −0.3085 | −0.1415 | −0.8205 | −0.1235 | 0.1185 | −0.0495 | −0.2535 | 0.6425 | 0.3755 |
| FBXL20 | −0.011 | 0.77 | −0.813 | −0.923 | −1.366 | −0.773 | 0.204 | 0.074 | 0.077 | 0.224 | −0.569 | −0.012 | −0.368 | −0.491 | −0.021 |
| FBXO25 | −0.023 | 0.111 | 0.608 | 0.045 | 0.172 | 0.401 | −0.235 | −0.197 | −0.258 | −0.131 | −0.319 | 0.011 | −0.155 | 0.272 | 0.035 |
| FDXR | 0.595 | −0.643 | −0.45 | 0.82 | 0.561 | 1.306 | 2.243 | −0.345 | −0.454 | −0.243 | −0.505 | −0.4 | 0.039 | 1.775 | −0.033 |
| FEM1B | −0.523 | −0.026 | 0.229 | −0.169 | 1.533 | 0.628 | 1.077 | −1.318 | −0.034 | −2.078 | −1.481 | −1.154 | 1.589 | −1.386 | −2.514 |
| FGF12 | 2.0185 | 1.5085 | 1.3085 | 1.0925 | 0.2395 | 0.5755 | 1.0416 | 0.5975 | 0.6265 | −0.0715 | 0.0925 | 0.3695 | 0.0495 | −0.6885 | −1.1345 |
| FGF13 | 0.403 | 0.359 | 0.35 | −0.448 | 1.56 | 2.128 | 1.381 | 0.492 | −0.187 | −0.926 | 0.123 | −0.541 | −0.039 | −0.989 | −0.578 |
| FHOD1 | −0.1215 | 0.5545 | −0.2585 | 0.4295 | 0.4365 | 0.6285 | 1.2685 | 0.3095 | −0.1215 | 0.3605 | −0.4175 | 0.1655 | −0.2015 | −0.8475 | −0.1025 |
| FKBP11 | −0.4395 | −0.2975 | −0.4925 | −0.0005 | −1.1725 | 0.0425 | 0.0075 | 0.0005 | 0.0105 | −0.4585 | −1.5935 | −0.5485 | −1.6775 | −1.0915 | −0.9255 |
| FLJ10094 | −0.595 | −0.484 | −0.367 | −0.699 | −0.573 | −0.518 | −0.738 | 0.338 | −0.356 | −0.245 | −0.844 | −0.3 | −0.079 | −0.399 | −0.086 |
| FLJ10115 | −0.3795 | 2.0945 | 0.4116 | 0.6315 | 0.3065 | −0.0245 | −0.3576 | −0.1855 | −0.1955 | −0.1105 | 0.5315 | 0.7495 | 0.3915 | −0.0905 | 0.5185 |
| FLJ10159 | −0.073 | −0.837 | −0.164 | −1.234 | −0.473 | −0.182 | −0.325 | 0.454 | −0.055 | 0.157 | 0.134 | −0.384 | 0.597 | 0.428 | 0.165 |
| FLJ10204 | 0.558 | −0.034 | −0.472 | 0.398 | 0.359 | −0.13 | 0.329 | −0.332 | 0.322 | 0.029 | 0.534 | 0.685 | −0.305 | 1.412 | 2.133 |
| FLJ10275 | −0.2845 | −0.4405 | −0.7035 | 0.0005 | −0.8135 | −0.5975 | −1.4975 | 0.4365 | −0.0015 | −1.2095 | −1.3985 | −0.9175 | 2.3355 | −0.1545 | −0.1045 |
| FLJ10292 | −0.0315 | −0.6055 | −0.2935 | 0.3815 | −0.0765 | 0.2145 | −0.1575 | −0.1885 | −0.3425 | 0.8585 | −0.3295 | 0.2005 | 1.3815 | 0.3965 | 0.7285 |
| FLJ10324 | 0.1825 | 1.3535 | 0.0595 | 0.0885 | −0.1135 | −0.0025 | −0.4045 | −0.1465 | 0.0305 | 0.5325 | 0.5205 | 0.3975 | −0.3315 | 1.7685 | 0.7205 |
| FLJ10700 | −0.555 | −0.787 | −0.628 | −0.95 | 0.014 | −0.31 | −0.069 | 0.3 | −0.436 | −0.608 | −0.437 | 0.129 | −0.355 | 0.839 | 0.185 |
| FLJ10706 | 1.34 | 1.613 | 1.859 | 1.704 | 0.878 | 1.344 | 2.277 | 1.002 | −0.042 | −0.501 | 2.103 | 2.808 | 1.655 | 1.05 | 1.433 |
| FLJ10671 | −0.583 | −0.739 | 0.329 | 0.184 | −0.369 | −1.213 | 0.962 | −0.059 | −0.056 | −0.348 | 0.616 | 0.637 | 0.034 | 0.404 | −0.517 |
| FLJ10901 | −0.4365 | 0.1045 | 0.7695 | −0.3655 | −0.2465 | 0.2215 | 0.1685 | −0.3665 | 0.8825 | −0.5935 | −1.5725 | −1.8865 | −1.5445 | −3.0435 | −1.8105 |
| FLJ10918 | −0.095 | −0.386 | 0.026 | 0.054 | −0.441 | 0.13 | 0.304 | −0.928 | 0.079 | 0.26 | 0.114 | −0.833 | −0.235 | 0.507 | −0.401 |
| FLJ10980 | −0.4285 | −0.1965 | −0.3735 | −0.0075 | −0.0505 | −0.6455 | −0.8235 | −0.7105 | 0.1205 | −0.3465 | −0.6435 | −0.0665 | −0.1615 | 0.9275 | 0.7116 |
| FLJ11017 | 1.1275 | 0.1345 | 0.9835 | 1.5375 | 0.2265 | 0.0575 | −0.0515 | 0.0625 | 1.3895 | 0.7116 | 0.1425 | −0.0515 | −1.0915 | −0.9515 | −0.0965 |
| FLJ11088 | −0.3215 | −0.0735 | −0.5085 | −0.4325 | 0.4745 | −0.4205 | −0.1325 | 0.4825 | 0.1725 | 0.3895 | 0.7275 | 0.1705 | 0.9375 | −0.1655 | 0.1775 |
| FLJ11151 | −0.5355 | 0.1695 | −0.1475 | −0.3025 | −0.4485 | −1.4565 | −0.3515 | −0.7145 | 0.3155 | 0.2115 | 0.7645 | −0.1705 | −0.5505 | −1.0865 | −0.4885 |
| FLJ11267 | 0.2535 | −0.1745 | −0.0165 | −0.5215 | 0.1195 | −0.0875 | 0.2005 | 0.3355 | −0.0955 | 0.3525 | −0.5705 | −0.4905 | −0.0395 | −0.1385 | 0.5775 |
| FLJ11280 | 0.0855 | 0.1525 | 0.0905 | −0.5435 | 0.1805 | −0.0835 | 1.2185 | 0.7745 | 0.4725 | −0.0855 | −0.0695 | 0.9745 | 0.0125 | 0.6755 | 0.3485 |
| FLJ11508 | −0.3545 | −0.6895 | −0.0415 | −0.8595 | 2.1725 | 0.1075 | −1.5805 | −0.1745 | 1.4635 | −0.3675 | −0.5855 | −0.4705 | −0.0885 | 0.7185 | 0.3335 |
| FLJ12270 | −0.9485 | −0.5225 | −1.1015 | −0.9075 | −0.8685 | −0.4735 | −1.8815 | 0.6545 | 0.4505 | 0.0235 | −0.5825 | −0.8925 | 0.6425 | 0.8445 | 0.7015 |
| FLJ12844 | 0.0545 | −0.1825 | −0.2335 | −0.0965 | 0.8255 | −0.3945 | 0.1855 | −0.3445 | 0.0435 | 0.2045 | 0.0075 | −0.2875 | 0.0235 | 0.8275 | 0.8485 |
| FLJ12650 | 0.5055 | −0.0785 | −0.1315 | 0.0385 | 0.1565 | −0.2585 | −0.3815 | 0.1335 | 0.1145 | 0.1615 | 0.6825 | 0.3725 | 0.5185 | 0.5515 | 0.3805 |
| FLJ12684 | 1.0795 | 0.1185 | −0.4835 | 0.8525 | −0.4915 | 0.3055 | 0.3545 | −0.0745 | 0.3185 | 0.0305 | 0.2845 | 0.4975 | −0.2555 | 0.2775 | 0.1645 |
| FLJ12735 | −0.021 | −1.246 | −0.796 | −1.818 | −0.26 | −0.08 | −0.868 | −0.204 | −0.264 | −1.429 | −0.706 | −0.412 | −0.819 | 0.404 | −0.13 |
| FLJ12750 | −2.027 | −0.4 | −0.757 | −1.95 | −0.361 | −2.202 | −0.977 | −1.421 | 0.653 | −1.168 | −1.623 | −2.898 | −3.697 | −2.052 | −2.338 |
| FLJ12895 | 3.3535 | 1.3855 | −0.0835 | 0.5705 | 0.8275 | −0.0735 | −0.3535 | 0.3175 | 0.2415 | −0.3776 | 0.3295 | 0.2465 | −0.2855 | 0.0275 | 0.1915 |
| FLJ13710 | 0.1265 | 0.1005 | 0.0875 | 1.3665 | 1.0516 | −0.6475 | −0.0745 | −0.5705 | −0.5355 | −0.3015 | 0.5795 | −0.0135 | −0.2815 | 0.4025 | 2.0485 |
| FLJ13855 | 0.19 | 0.44 | 0.334 | −0.887 | −0.806 | −0.406 | 0.302 | 0.141 | 0.562 | 0.089 | 0.949 | 1.099 | 1.133 | 0.9 | 0.245 |
| FLJ13912 | −0.835 | 0.7 | −0.208 | −0.803 | 0.650 | −0.892 | −0.121 | −0.21 | −0.235 | 0.003 | 1.825 | 0.196 | −0.212 | 2.105 | 0.036 |
| FLJ14124 | 0.198 | −0.134 | −0.237 | 0.494 | 0.554 | 0.411 | 0.220 | −0.399 | −0.091 | −0.135 | 0.556 | 0.326 | 0.011 | 0.798 | 0.775 |
| FLJ14627 | 0.0505 | −0.1355 | −0.1085 | 0.6765 | −0.2385 | −0.3045 | 0.2025 | −0.1295 | 0.2355 | 0.0005 | −0.3925 | −0.1675 | 0.2475 | −0.1255 | −0.2255 |
| FLJ14666 | −0.818 | −0.873 | 0.124 | −0.277 | 1.568 | 0.396 | 0.014 | 1.008 | −1.009 | −0.412 | −0.223 | −0.588 | −0.138 | −0.593 | −0.213 |
| FLJ14904 | −2.17 | −1.225 | −1.78 | −1.478 | −1.089 | −2.302 | −2.128 | −0.286 | −0.748 | −0.827 | −1.295 | −1.077 | −0.934 | −2.089 | −1.4 |
| FLJ20035 | 0.572 | −0.013 | −0.044 | 0.937 | 2.123 | 0.732 | 0.997 | −0.493 | −0.157 | 0.001 | 0.257 | −0.12 | −0.253 | 0.357 | −0.127 |
| FLJ20152 | 0.303 | −0.187 | −0.489 | −0.341 | −0.949 | −0.519 | −0.447 | 0.823 | 0.489 | 0.1 | 0.323 | 0.392 | 0.587 | 0.164 | 2.361 |
| FLJ20171 | −0.818 | −0.747 | −0.382 | −0.252 | 0.075 | −0.153 | −0.39 | 0.393 | −0.181 | −0.451 | −1.482 | −0.82 | −0.721 | −1.233 | −0.377 |
| FLJ20244 | 0.151 | −1.578 | −0.389 | −1.189 | 0.395 | −0.902 | −1.151 | 1.487 | −0.844 | −0.318 | −1.69 | 0.051 | −0.782 | −1.237 | −0.313 |
| FLJ20273 | 0.262 | 0.264 | 0.369 | 0.137 | 0.21 | −0.028 | 0.157 | −0.002 | 0.018 | −0.228 | 0.058 | −0.818 | −0.128 | −0.13 | −0.006 |
| FLJ20315 | 0.728 | 0.927 | 0.118 | −0.007 | 0.196 | −0.261 | −0.152 | −0.65 | 0.081 | 0.007 | −0.001 | −0.529 | 0.277 | 0.687 | 1.668 |
| FLJ20323 | −1.0985 | −0.1185 | 0.1255 | 0.1465 | 2.0875 | 0.4885 | 0.3865 | −0.0225 | −0.1615 | 0.2945 | −0.1445 | 0.0495 | 0.4855 | −0.0555 | −0.2155 |
| FLJ20518 | −0.918 | −0.719 | −0.379 | −0.356 | 0.381 | 0.82 | 0.242 | 1.63 | 0.227 | −0.65 | −0.302 | 0.171 | 0.253 | 0.69 | −0.346 |
| FLJ20530 | 0.586 | 0.773 | 0.5 | 0.811 | 0.656 | 0.218 | 0.494 | 0.039 | 0.237 | −0.096 | 0.216 | −0.286 | −0.118 | 0.083 | 0.357 |
| FLJ20696 | −0.8755 | −0.7105 | 0.7365 | −0.4815 | 0.4755 | 0.1895 | −1.1495 | 0.0265 | −0.4275 | 0.0915 | −0.0565 | 0.5555 | 0.5895 | −0.0815 | 0.2585 |
| FLJ20718 | −0.44 | −0.615 | 0.002 | −0.246 | −0.085 | −0.018 | −0.744 | 0.901 | 0.265 | 0.219 | 0.208 | 1.117 | 0.663 | 0.285 | 0.486 |
| FLJ20772 | −0.0635 | −0.7285 | −0.1895 | −0.5725 | −0.8695 | −0.8905 | 0.0405 | 0.5505 | −0.0785 | −0.5985 | −0.1085 | 0.1825 | −0.4045 | 0.3925 | 0.1595 |
| FLJ20989 | −1.2345 | −0.9635 | −0.8915 | −0.6775 | −0.5095 | −0.9915 | −0.6725 | 0.4555 | 0.3135 | −0.8145 | −1.4825 | −0.2855 | −1.3035 | −0.4445 | −0.7785 |
| FLJ21019 | −0.8775 | 0.2405 | 0.9115 | −0.0135 | 0.5225 | −0.2075 | −0.6155 | 0.0695 | −0.3785 | −0.3805 | −1.3345 | −0.3795 | −2.1285 | 0.5615 | 0.5845 |
| FLJ21062 | −0.8425 | −1.3825 | −1.6995 | −1.8315 | 1.1455 | −0.9965 | −0.1455 | 0.5985 | 0.0945 | −0.4385 | −1.2295 | −0.3315 | 0.3665 | 0.5375 | −0.8005 |
| FLJ21159 | 1.0115 | −0.0495 | 0.3615 | 0.3145 | −0.4485 | −0.0105 | −0.2895 | −0.1555 | 0.5295 | −0.1765 | −0.1145 | −0.3085 | −1.4435 | 0.0085 | −0.7405 |
| FLJ21616 | 2.818 | −0.059 | 0.676 | −1.103 | −0.853 | 0.841 | −0.597 | −0.964 | 0.889 | −0.417 | −0.838 | −1.18 | −0.607 | −2.533 | −1.694 |
| FLJ21827 | −0.1475 | 0.3835 | 0.5815 | 0.4475 | 1.9935 | 0.9145 | 0.0315 | −0.1055 | 0.2095 | −0.7145 | −0.8015 | −0.7525 | −1.4055 | −0.4535 | −0.9175 |
| FLJ21963 | 1.122 | −0.078 | 1.327 | −0.295 | −0.243 | 1.117 | −0.12 | −0.496 | −0.512 | −1.14 | −0.31 | −0.254 | −0.641 | −0.269 | 0.153 |
| FLJ22104 | 1.2085 | −0.1795 | −0.5676 | 0.2525 | 0.5885 | 0.6625 | 1.2575 | 1.6855 | 0.2925 | 0.2215 | 0.5805 | 0.8855 | 0.5645 | −0.3505 | −0.3295 |
| FLJ22222 | 1.371 | −0.107 | 0.325 | 0.373 | 0.58 | 0.239 | −0.257 | 0.35 | 0.164 | −0.464 | −0.23 | −0.489 | −0.346 | −0.081 | −1.227 |
| FLJ22573 | −0.2865 | −0.8965 | −0.2105 | 0.0075 | −0.6875 | −0.4505 | 0.7905 | −1.3385 | −0.5355 | −0.3825 | −1.2945 | −0.9575 | −0.3245 | 0.4415 | −1.0725 |
| FLJ22794 | −0.812 | −0.326 | −0.31 | −0.155 | 0.276 | 0.434 | −0.274 | 0.092 | 0.386 | −0.583 | −0.596 | 0.448 | −0.395 | 1.358 | 0.681 |
| FLJ23188 | −0.4395 | −0.1605 | 0.7715 | 0.3775 | −0.0375 | −0.6185 | 1.1685 | 0.1515 | −0.2485 | −0.2235 | −0.2385 | 1.3165 | 0.1165 | −0.4015 | 2.2415 |
| FLJ23441 | 0.944 | −0.698 | −0.291 | −1.21 | −0.432 | −0.005 | −0.275 | −1.176 | 0.095 | −0.56 | −0.514 | −1.123 | −0.267 | −0.724 | 1.013 |
| FLJ25471 | 2.843 | −0.485 | −0.102 | 0.382 | −0.08 | −0.579 | 0.063 | 0.222 | 0.091 | −0.259 | 0.323 | 0.96 | −0.975 | 0.184 | 0.002 |
| FLJ31204 | 0.5935 | 0.4215 | 0.3785 | 0.3235 | 0.4745 | 0.3895 | −0.7515 | −0.7695 | 0.4885 | 0.2835 | 0.6035 | 2.1885 | 0.6525 | −0.0875 | 2.1635 |
| FLJ31795 | 1.2765 | −0.7995 | −0.3915 | −0.9025 | −0.3865 | 0.6345 | 1.7915 | 2.2565 | 0.6635 | −0.0755 | 0.6765 | 0.9825 | 1.4025 | −0.6925 | −0.3845 |
| FLJ32942 | −1.7165 | −1.9515 | −1.4185 | −2.3275 | 0.2275 | −0.1375 | −2.1885 | −1.1835 | −1.4715 | −1.2385 | −2.7855 | −2.1465 | −2.3855 | −1.3825 | −0.5285 |
| FLJ37970 | −2.523 | −0.601 | 0.942 | −2.797 | −1.027 | 2.126 | −2.475 | −1.808 | 0.744 | 2.369 | 2.163 | 1.882 | 0.208 | −0.161 | −0.151 |
| FLJ39370 | 0.2795 | −0.2375 | −0.2015 | 0.1105 | 0.1825 | 1.5515 | −0.3935 | 0.3025 | 0.2435 | −0.5725 | −0.3355 | −0.2035 | −1.0545 | −0.4045 | −0.3155 |
| FLJ90588 | 0.2715 | −0.1315 | 2.0035 | 0.3435 | 0.3545 | 0.6135 | 1.3955 | −0.9245 | 0.8145 | −0.0965 | 0.7105 | 0.5165 | 0.6255 | 0.7845 | 1.7145 |
| FN3KRP | −0.806 | −0.286 | −0.057 | −0.878 | 0.107 | −0.543 | −1.145 | −1.363 | −0.208 | −0.45 | −6.002 | −4.471 | −4.048 | −4.336 | −8.128 |
| FOLR1 | −0.5095 | −0.6295 | −0.0795 | 1.3005 | −1.0995 | −0.8095 | −0.3095 | 0.3305 | 0.2115 | −0.8095 | 3.1605 | 2.5705 | 2.7605 | 4.0265 | 3.4705 |
| FOXA1 | −0.6555 | −0.4075 | 0.1535 | −0.3195 | −0.1735 | −0.3675 | −0.5375 | 0.4645 | −0.1885 | −0.4995 | −0.4985 | −0.3045 | −0.3025 | −1.1425 | −0.3645 |
| FOXC1 | −0.048 | 0.2 | 0.775 | 1.661 | 0.026 | 0.483 | 1.393 | 0.738 | −0.393 | −0.155 | −0.258 | 0.282 | 0.33 | 1.251 | 0.311 |
| FOXP1 | −0.4245 | 0.0895 | −0.1905 | −0.2265 | −0.2425 | −0.3555 | 0.2045 | 0.6375 | 0.3905 | −0.3635 | −0.3325 | −0.1085 | −0.5845 | 0.3255 | 0.8155 |
| FREQ | −0.5005 | 0.3875 | 0.3015 | 0.5835 | −0.0305 | 0.0115 | −0.3405 | −0.4485 | 0.0055 | −0.4775 | 0.5865 | 0.0865 | −0.3305 | 0.4885 | 0.0115 |
| FTO | 0.7975 | −0.7815 | −0.5485 | −0.4515 | 0.2355 | 1.0645 | 0.7295 | 0.1645 | −0.9795 | −0.6005 | −1.6395 | −0.4865 | −0.6345 | −0.3865 | −0.7405 |
| FUBP1 | −0.6475 | −0.9025 | 0.0025 | −0.9125 | −0.2655 | 0.2445 | −0.5915 | 0.3055 | 0.0155 | −0.0535 | −1.2645 | −1.2565 | −1.3345 | −0.8245 | −1.5415 |
| FUCA1 | 0.094 | −0.275 | 0.941 | −0.73 | −0.069 | −0.259 | 0.26 | 0.744 | −0.895 | −0.755 | 0.88 | 0.147 | 1.076 | 0.782 | 0.298 |
| FUT8 | −0.1855 | −0.2295 | −0.0555 | 0.5315 | −0.1945 | 0.1635 | 0.1325 | −0.1875 | −0.1875 | 0.1485 | −0.8555 | −0.5295 | −1.4325 | −0.9455 | −0.3555 |
| FXYD6 | −0.342 | −0.036 | 0.169 | −0.104 | −0.359 | 0.486 | −1.334 | −0.304 | 0.226 | 0.633 | −0.014 | −0.514 | 0.119 | −0.975 | 1.843 |
| FYCO1 | −0.999 | −0.119 | 0.496 | −0.076 | −0.136 | −0.369 | −0.284 | −0.031 | 0.319 | −1.064 | −1.111 | 1.343 | 1.058 | 1.001 | 2.263 |
| FZD10 | 0.0055 | −0.2595 | 0.0625 | 0.3735 | 0.0175 | 0.6295 | −0.3445 | −0.5405 | −0.2655 | −0.0545 | 1.4135 | 0.3445 | 2.3645 | 1.0725 | 1.5165 |
| FZD7 | 1.3195 | 0.3485 | 1.6775 | −0.3015 | −0.2015 | −0.4195 | 0.3815 | 0.9805 | −0.7515 | 0.2885 | 1.5455 | 0.8055 | 1.5035 | 0.0275 | 1.7515 |
| FZD9 | 1.0895 | 0.3075 | 1.5735 | 0.0965 | −0.4065 | −0.0975 | −0.1195 | 1.0175 | −0.4945 | 0.1625 | 1.2955 | −0.5495 | 1.5995 | 0.1535 | 0.5025 |
| G1P2 | 0.0415 | 1.2005 | 0.0865 | 0.7265 | −0.1485 | 0.1615 | −0.7585 | −0.0735 | −0.3845 | −0.1285 | 0.1045 | −0.6145 | −0.8205 | 1.0285 | 0.8985 |
| G1P3 | −0.594 | −0.039 | 0.499 | −0.202 | 1.093 | −0.325 | −0.038 | −0.054 | −0.411 | −0.666 | 0.009 | −0.063 | −0.514 | −0.98 | −0.195 |
| GA17 | 0.6325 | 1.1735 | −0.0345 | 0.3945 | 1.4695 | −0.8735 | −0.2535 | −0.7045 | −1.6385 | −0.4725 | 0.2835 | 0.0785 | −0.4955 | 1.3075 | 1.3185 |
| GABARAP | 1.3655 | 0.5865 | 0.4585 | 0.1905 | 0.2845 | 0.6285 | 0.3495 | 0.4015 | 0.9845 | 0.7875 | −0.3915 | −0.8905 | −0.2025 | 0.2785 | −1.0915 |
| GAJ | 0.5075 | 2.8835 | 2.8795 | 2.0345 | 1.6615 | −0.7705 | 2.3845 | −0.6745 | −0.8575 | −0.7235 | −0.4905 | −0.0285 | 3.1335 | 2.1555 | 0.9255 |
| GALE | 0.822 | 0.588 | 0.925 | 0.269 | 0.84 | 0.385 | 1.205 | −0.033 | 0.648 | 0.724 | 0.6 | −0.117 | 0.715 | 0.51 | 0.493 |
| GALNT14 | −0.7875 | 0.1935 | −0.0885 | 0.4575 | 1.4415 | −0.0985 | 0.1815 | −0.8305 | −0.2745 | −0.9015 | −1.8865 | −2.4925 | −1.1955 | −1.4675 | −0.9155 |
| GALNT2 | 1.9915 | 0.5515 | −0.5265 | −0.4885 | −0.2705 | −0.4515 | −1.7535 | −0.4735 | 0.0405 | 0.9205 | −0.5235 | −0.4025 | −0.0065 | −1.7675 | −1.5115 |
| GALNT7 | 0.701 | 0.137 | −0.307 | 0.279 | 0.085 | 0.266 | 0.637 | −0.178 | 0.118 | 0.18 | 0.737 | 1.455 | 0.044 | 1.399 | 2.323 |
| GAMT | 0.119 | 0.395 | 0.869 | 0.955 | −0.102 | 0.081 | 0.118 | 0.099 | −0.175 | 0.499 | 0.912 | 0.62 | 0.563 | 1.074 | 0.563 |
| GAPD | 0.652 | −0.09 | 0.009 | 0.808 | 0.223 | −0.167 | −1.022 | 0.276 | 0.058 | 0.164 | 0.517 | 0.757 | 0.107 | 0.996 | 0.948 |
| GARS | −2.942 | −0.12 | −0.998 | −3.279 | −0.591 | −1.702 | −1.874 | 0.21 | −0.288 | 0.035 | −2.883 | −3.054 | −2.15 | −0.817 | −1.135 |
| GART | 2.0455 | −2.0845 | 0.6015 | 1.1645 | −1.5475 | −0.0925 | 1.2955 | −0.0915 | 1.0095 | −0.2635 | −1.8305 | −1.2115 | −1.2885 | −0.6435 | 0.4665 |
| GATA3 | 1.0305 | 0.5825 | 0.5215 | 0.8575 | −0.2345 | 0.2975 | 0.6035 | 0.1855 | 0.4135 | 1.4715 | 0.9545 | 0.9515 | 1.5485 | 0.3225 | −0.3765 |
| GATM | −0.784 | −0.527 | 0.166 | 0.51 | −0.821 | −0.624 | −1.532 | 2.171 | 0.445 | −0.69 | −0.208 | −0.532 | −0.912 | 0.141 | 0.462 |
| GBA | 0.663 | 0.592 | 0.541 | 1.256 | 0.462 | 0.176 | 0.526 | 0.335 | −0.187 | 1.208 | 0.495 | 0.633 | −0.211 | 0.888 | 2.064 |
| GDAP1 | 0.461 | 0.048 | −0.178 | −0.903 | −0.428 | −0.178 | 0.762 | 0.585 | −0.082 | −0.123 | 0.364 | 0.85 | 0.415 | 0.068 | −0.259 |
| GDI2 | −0.3935 | −0.0595 | −0.6085 | −1.3745 | 0.0775 | −0.3705 | −0.6675 | 0.1785 | 0.0315 | −0.6045 | 0.0065 | −0.4625 | −1.0585 | −0.7985 | 1.0275 |
| GGA1 | −1.7865 | −0.4125 | −1.1125 | −0.4145 | 0.8285 | 1.1745 | 0.4515 | 0.6245 | 0.2215 | 0.2085 | −1.2035 | −1.2055 | −0.3585 | −1.1055 | −0.5335 |
| GKAP1 | 0.4485 | −0.2025 | 0.8765 | −0.2335 | 0.5875 | 0.2075 | 1.4995 | 0.5116 | 0.0355 | −0.0665 | 0.4025 | 0.6375 | 0.0215 | 0.2585 | 0.0685 |
| GLCCI1 | −0.0955 | 0.4045 | −0.5825 | −0.5345 | −1.1595 | −0.0545 | −0.4415 | 0.3545 | −0.0175 | 0.3695 | 0.1795 | 0.8825 | −0.1745 | 0.1805 | −0.6765 |
| GLG1 | 0.601 | −0.063 | 1.188 | 1.494 | 0.457 | 0.62 | −0.523 | −0.515 | −0.297 | 0.535 | 0.649 | −0.116 | −0.104 | 0.324 | 0.387 |
| GLTSCR2 | −1.233 | 0.551 | 0.657 | −0.95 | 0.288 | −0.018 | 0.147 | 0.212 | 0.403 | 0.459 | −0.706 | −1.427 | 0.128 | −0.356 | −0.473 |
| GNB4 | −0.091 | −0.165 | −0.321 | −0.452 | −0.276 | −0.428 | −0.037 | 0.418 | 0.308 | 0.439 | 0.319 | −0.091 | 0.106 | 0.491 | −0.334 |
| GOLPH2 | 0.4475 | 0.2895 | 0.5735 | 0.8975 | 0.6835 | 0.1615 | 1.2725 | 0.4965 | 0.3605 | 0.1415 | 0.9605 | 0.7235 | 0.5775 | 1.2825 | 1.1295 |
| GPATC1 | −0.6055 | −0.9175 | −0.5345 | −1.7635 | 0.4825 | −0.7605 | −0.1585 | −0.1655 | 0.2725 | −2.1075 | 0.3545 | 1.4385 | 2.0005 | 1.2805 | 1.4725 |
| GPC2 | −1.3945 | −0.4135 | 0.4255 | −0.0665 | −0.0045 | −0.2255 | −0.8355 | −0.0095 | −0.1595 | −0.2425 | −1.3695 | −2.6785 | −1.2355 | −1.6415 | −2.1995 |
| GPR125 | −0.387 | 0.694 | 1.368 | 0.305 | 1.065 | −0.11 | 1.005 | 0.504 | 0.151 | 0.326 | 1.268 | 0.611 | 0.726 | 0.538 | 0.875 |
| GPR160 | 0.6435 | 1.5435 | 0.0985 | 0.2875 | −0.7225 | 0.4835 | −1.9715 | 0.2755 | 0.1755 | −0.7685 | −0.6905 | −1.2035 | −0.5385 | −1.3985 | −0.0915 |
| GPR56 | −0.0695 | 0.0035 | 0.4995 | 0.4255 | 0.1465 | −0.0445 | −0.0245 | 0.5495 | −0.2345 | 0.0145 | 0.7825 | 0.1835 | 1.1375 | −0.0855 | −0.0255 |
| GPRC5C | 0.5325 | 0.1865 | 0.1595 | 0.9115 | −0.2165 | 0.2225 | 0.4965 | −0.0135 | −0.6925 | 0.1075 | 1.4935 | 1.6025 | −0.0275 | 1.3205 | 1.6405 |
| GPSM1 | −0.598 | −0.422 | 0.096 | −0.287 | −0.137 | −1.199 | 0.327 | 0.879 | 0.342 | −0.803 | 0.555 | −0.218 | −0.667 | 0.354 | 0.888 |
| GPSM2 | 1.091 | −0.155 | −0.581 | 1.081 | 1.006 | 0.567 | 0.518 | −0.198 | 1.152 | 0.372 | 1.51 | −0.044 | 0.523 | 1.333 | 1.162 |
| GPT | −0.3225 | −0.1695 | 0.8885 | 0.7925 | 0.4775 | 0.0015 | −0.2205 | −0.0325 | −0.2205 | −0.3075 | 0.3115 | 0.0825 | 0.9025 | 0.9725 | 1.8545 |
| GPT2 | −0.728 | −1.499 | −0.008 | −0.795 | −0.838 | −1.065 | −0.067 | 0.585 | 0.103 | −0.707 | 1.663 | 0.792 | 2.945 | 1.186 | −0.281 |
| GPX7 | 1.391 | 1.539 | 1.743 | 0.839 | 2.115 | 1.951 | 1.834 | −0.049 | −0.058 | 0.37 | 0.365 | 1.04 | 0.65 | −0.071 | −0.553 |
| GRB14 | −1.0465 | −0.2315 | −0.7885 | 0.5845 | −0.1375 | 0.1835 | −0.7415 | 0.8055 | −0.7085 | −0.5145 | −1.1215 | −0.3585 | −0.2175 | 0.4535 | −1.1805 |
| GRB7 | 1.9695 | 0.2575 | 1.5355 | 0.1655 | 0.2685 | 2.2155 | 2.5235 | 1.0175 | 0.3005 | 1.1765 | 0.6405 | 1.0495 | 1.5635 | 0.5045 | −0.5985 |
| GSC | −0.35 | −0.42 | 0.235 | 0.154 | −0.482 | 0.191 | 1.13 | 0.125 | −0.042 | 0.002 | −0.05 | 0.553 | 0.023 | 0.626 | −0.13 |
| GSDML | 0.37 | 0.127 | 0.714 | 0.001 | 0.039 | −0.647 | 0.162 | −0.261 | −0.152 | −0.224 | 0.079 | 1.38 | 0.899 | 0.518 | 0.061 |
| GSK3B | 1.037 | −0.674 | −0.288 | −0.122 | −0.666 | −0.071 | 0.329 | 0.193 | −0.19 | −0.071 | 0.273 | −0.003 | −0.208 | −0.793 | −0.914 |
| GSTA1 | 1.5315 | −0.2785 | −0.6525 | 0.3465 | −1.5175 | 1.4165 | −0.0855 | 0.9455 | 0.5305 | 1.0875 | −2.6435 | −1.8755 | −1.9965 | −1.1805 | −2.8885 |
| GSTK1 | 0.106 | 0.422 | −0.011 | −1.447 | −1.265 | −0.599 | −0.369 | −0.288 | −0.588 | −1.125 | 1.407 | 1.388 | 0.936 | 1.452 | 2.188 |
| GSTM3 | 0.5705 | 0.4805 | −0.2495 | 0.6635 | −1.6665 | 0.3515 | −0.6925 | −0.0625 | −0.1845 | −1.1605 | 0.2645 | 0.8485 | 0.0565 | −1.5725 | −0.4676 |
| GSTP1 | −0.168 | −0.242 | 1.053 | 0.754 | −0.441 | −1.57 | 0.523 | −0.173 | 1.348 | −0.599 | −0.153 | 0.884 | −0.68 | −0.094 | −0.847 |
| GSTT1 | −0.9675 | 0.8125 | −0.2715 | 0.5755 | 0.0445 | −0.4195 | −0.0485 | −0.0285 | −0.7055 | −0.3145 | −0.2495 | 0.5225 | 0.5065 | 0.2275 | 1.3595 |
| GSTT2 | −0.3825 | −0.8685 | −0.4905 | −0.2885 | 0.4315 | −0.1885 | 0.2575 | −0.1875 | 0.1815 | 0.4495 | −0.2185 | 0.0325 | 0.8505 | 0.1005 | 0.3325 |
| GTF2F2 | −0.663 | 0.848 | −0.338 | 0.739 | 0.145 | 0.006 | 0.478 | 0.214 | 0.188 | −0.428 | −0.515 | 0.166 | −0.307 | −0.478 | 0.878 |
| GTF2I | 0.1445 | −0.8085 | −0.6385 | −0.9525 | −1.2155 | −0.8315 | −0.1325 | 0.5285 | −0.2155 | 1.2495 | −0.1585 | 0.0285 | 0.6495 | 0.2455 | 1.6225 |
| GTF3A | −0.008 | 0.408 | 0.08 | 0.996 | 0.007 | −0.004 | 1.304 | −0.204 | 0.434 | 1.117 | 0.365 | 1.21 | 0.325 | 2.321 | 0.639 |
| GTPBP3 | −0.515 | 0.468 | 0.808 | −0.383 | −0.002 | 0.834 | 1.43 | −0.177 | −0.728 | −0.026 | 1.708 | 0.677 | 0.594 | −2.919 | −1.558 |
| GTPBP4 | 0.301 | −0.301 | 0.4 | 0.354 | 0.694 | 0.187 | −0.044 | −0.267 | 0.043 | 0.041 | −0.172 | −0.299 | −0.139 | 0.858 | 0.325 |
| H1F0 | −0.0975 | −0.4925 | −0.1705 | −0.2355 | 0.4525 | −0.0315 | 0.3115 | 0.2265 | −0.2025 | −0.0995 | 0.0105 | 0.8845 | −0.0815 | 0.1145 | −0.8135 |
| H2AFV | 0.1145 | 0.0545 | 0.4265 | 0.3145 | 1.8445 | 0.3765 | −0.5215 | −0.7685 | −0.8655 | −0.0835 | −0.2205 | −0.2345 | −0.2105 | 1.1385 | 0.3315 |
| H2AFY | −0.7705 | 0.2575 | 0.3885 | 0.6375 | 1.1685 | 0.5725 | 0.3165 | −0.3305 | −0.3655 | −1.7385 | 0.5935 | −0.5635 | 0.6845 | 1.0175 | −0.1715 |
| H2AFZ | −0.5725 | −0.2785 | 3.2795 | 0.7515 | 0.3805 | 0.2515 | 0.9405 | −0.1815 | −1.0035 | −0.9335 | −0.4915 | −0.2265 | −1.2215 | −0.4985 | 0.1155 |
| H2BFS | 0.1225 | −0.1375 | −0.0725 | 0.8305 | 0.2075 | 0.3405 | 0.5155 | 0.0085 | −0.0265 | −0.3995 | 0.8885 | −0.1155 | −0.5765 | −0.0105 | −0.4075 |
| H3F3B | 0.684 | −0.28 | −0.124 | −0.098 | −0.011 | −0.372 | −0.032 | −0.045 | 0.454 | 0.475 | 0.182 | 0.412 | −0.049 | −0.641 | 0.288 |
| H41 | 0.4605 | −0.4105 | 0.0475 | −0.2235 | −0.0895 | 0.1215 | −1.3425 | 0.0585 | 0.0785 | −0.1635 | 0.3355 | −0.4795 | 0.5365 | 0.2645 | −0.4135 |
| HAGH | 0.714 | 0.101 | −0.027 | 0.993 | 0.309 | −0.02 | −0.117 | −0.463 | 0.282 | −0.338 | −0.293 | −0.55 | −1.11 | 0.257 | −0.48 |
| HBG1 | 0.6185 | 1.0545 | −0.1475 | −0.1225 | 0.6305 | −0.2075 | −0.6165 | 0.1315 | −0.2925 | 0.3585 | 1.4655 | 0.9265 | 0.5325 | 1.7855 | 1.1795 |
| HBL01 | −0.629 | −0.396 | 0.164 | −0.11 | 1.718 | 0.732 | −0.162 | 1.471 | −0.229 | 0.554 | 0.884 | 0.609 | 0.293 | 0.153 | 0.641 |
| HCAP-G | 0.3205 | 0.4805 | 0.7785 | 0.0785 | 0.0245 | 0.1205 | −0.1775 | 0.0905 | 0.1105 | 0.4195 | 0.8115 | 0.5485 | −0.6595 | 1.5665 | 2.2446 |
| HCP5 | 0.264 | −0.091 | 0.435 | −0.058 | −0.389 | −0.177 | −0.479 | 1.323 | 1.056 | −0.602 | −0.052 | −0.006 | −0.48 | −0.578 | −0.413 |
| HDAC2 | −0.376 | −0.793 | −1.341 | −0.725 | −0.16 | −0.182 | −0.375 | −0.408 | 0.022 | −0.375 | −1.592 | −0.091 | −0.095 | −0.019 | −0.224 |
| HDC | −0.2305 | 0.0395 | −0.1995 | −0.2645 | −0.0475 | 0.2015 | 0.2855 | −0.0645 | 0.1285 | −0.0005 | −0.2385 | −0.0255 | 0.3245 | −0.0435 | −0.3655 |
| HEBP1 | 0.291 | −1.451 | −0.711 | −0.627 | −0.855 | −0.248 | 0.489 | 0.484 | 0.091 | −0.048 | −0.733 | 0.596 | 0.602 | 0.437 | −0.407 |
| HIPK2 | −0.128 | 0.068 | 0.447 | −0.674 | 0.64 | −0.709 | 0.621 | −0.441 | −0.905 | −0.04 | −0.439 | −0.579 | −1.718 | −0.54 | −0.752 |
| HIPK2 | −0.3885 | 1.1605 | 0.8165 | 1.2075 | 0.8455 | −0.0335 | 1.2355 | −0.8805 | −0.5815 | −1.7375 | 0.5815 | −0.9005 | 1.3295 | −0.0565 | −0.6855 |
| HIS1 | −1.1045 | 0.1345 | 0.0975 | 0.3075 | 1.0115 | 0.5135 | 0.4215 | −0.1005 | −0.5285 | −1.6035 | 0.6355 | −0.5415 | 1.1685 | 0.4955 | −0.8585 |
| HIST1H2A | −1.046 | 0.152 | 0.16 | 0.298 | 1.141 | 0.498 | 0.374 | −0.139 | −0.174 | −1.558 | 0.994 | −0.642 | 1.18 | 0.5 | −0.788 |
| HIST1H2B | −0.9775 | 0.1875 | 0.1775 | 0.3125 | 1.2095 | 0.5745 | 0.3705 | −0.1915 | −0.0455 | −1.5695 | 0.9075 | −0.6735 | 1.2875 | 0.3705 | −0.8985 |
| HIST1H2B | −0.91 | 0.053 | 0.118 | 0.231 | 1.056 | 0.511 | 0.234 | −0.058 | −0.269 | −1.821 | 0.871 | −0.478 | 1.173 | 0.38 | −0.783 |
| HIST1H2B | −1.1825 | −0.4385 | 2.9215 | 0.2965 | −0.1605 | 0.1705 | 0.8945 | −0.3725 | −0.9955 | −0.8915 | −1.2715 | −0.5045 | −1.1985 | −0.4775 | 0.1225 |
| HIST1H2B | 1.7225 | 0.3185 | 0.6275 | 0.0025 | 0.3725 | 1.3885 | 0.0665 | 0.2275 | 0.7535 | 0.7965 | 0.6315 | −0.0675 | 0.4945 | 0.0925 | 0.6715 |
| HIST1H3C | 0.7876 | 0.2735 | 0.1025 | 1.0635 | 0.9085 | 1.4605 | 0.8725 | 0.7765 | −0.3555 | −0.6075 | 0.2025 | 0.3915 | 0.0135 | 1.0335 | −0.0175 |
| HM13 | 0.5565 | 2.9975 | 0.7985 | 1.5675 | 0.5025 | 1.0555 | 2.3355 | 0.1575 | 0.1025 | 0.3625 | 0.1415 | −0.5225 | −0.4575 | 1.4425 | −0.0465 |
| HMGB3 | 0.0835 | 0.5315 | 0.2175 | 0.5355 | 1.0725 | −0.3035 | 0.2345 | −0.2785 | −0.4495 | 0.3815 | 0.3935 | 0.1525 | −0.5375 | 0.4976 | −0.0215 |
| HN1 | −0.582 | 0.026 | −0.16 | −0.468 | 0.475 | −0.269 | −0.332 | −0.347 | −0.558 | −0.74 | −0.319 | −0.776 | −1.048 | −0.199 | 0.419 |
| HNRPA3P | −0.4985 | −0.2975 | −0.2445 | −0.3385 | −0.1045 | 0.3565 | −0.0265 | 0.4485 | −0.2635 | −0.0895 | −0.2715 | −0.1025 | 1.1695 | −0.6635 | −0.6735 |
| HNRPDL | −0.92 | −0.583 | −0.009 | −0.524 | −1.492 | 0.629 | 0.024 | 0.644 | −0.703 | −0.151 | −0.033 | 0.277 | 1.379 | −1.977 | −0.651 |
| HOXA5 | −2.4375 | −0.9435 | −1.3385 | 0.1905 | 1.2635 | −0.3665 | 0.0085 | 0.7495 | −0.9495 | −1.1495 | −1.1735 | −1.1195 | −1.4145 | −3.5655 | −2.4485 |
| HOXA7 | −0.0625 | 0.2425 | 0.5055 | 1.0875 | 1.3125 | −0.9375 | −0.3015 | 1.3215 | 0.0705 | 0.2035 | −0.0155 | −0.8545 | −0.1095 | −0.2165 | −0.7255 |
| HOXB2 | −1.2075 | −1.0765 | −0.7235 | 1.4435 | 1.0795 | −0.2345 | 0.1585 | 1.0035 | −1.2055 | 0.2015 | 0.6215 | 0.3845 | 0.4105 | −1.2275 | −1.0765 |
| HOXB6 | −0.2155 | 0.5745 | 1.0765 | −0.0435 | 0.2535 | −0.4695 | 0.1715 | 2.0005 | −0.5645 | 0.3615 | 0.8625 | 0.7785 | 0.2225 | 0.5365 | −0.0285 |
| HOXB7 | −0.1545 | 0.8845 | 0.6095 | 0.1455 | 0.9475 | 0.8675 | −0.2065 | 2.1865 | 0.0815 | 0.6615 | 0.3205 | −0.1825 | 0.4825 | 0.8165 | −0.1205 |
| HOXC10 | −1.267 | 0.263 | 0.306 | −0.122 | 0.07 | 0.113 | −0.02 | 0.643 | −0.414 | 1.033 | 0.593 | −0.195 | 0.05 | −0.654 | 0.076 |
| HOXC13 | −0.3585 | 0.1755 | 0.2435 | 0.2355 | −0.2175 | −0.8255 | 0.1925 | 0.8375 | 0.1985 | −2.0275 | 0.6875 | −0.0725 | −1.8795 | 0.1925 | 0.9535 |
| HOXC9 | −0.35 | −0.445 | −0.416 | 0.275 | −0.639 | −0.231 | −0.749 | 1.404 | −0.737 | −0.856 | −0.732 | −0.419 | −0.876 | −0.032 | 0.933 |
| HOXD3 | −0.5445 | −1.0695 | −0.4535 | 0.3025 | 0.4355 | 0.0235 | 0.0275 | 0.4135 | −0.0715 | −0.3105 | 0.2105 | −0.9315 | −0.3215 | 0.0485 | −0.5315 |
| HPCL2 | 0.225 | −0.058 | 0.882 | 1.943 | −0.258 | 0.188 | 0.787 | 1.484 | 0.144 | 0.603 | 0.797 | −1.32 | 1.304 | 1.68 | −0.376 |
| HPS3 | −0.0365 | 0.3685 | 0.4695 | 1.3045 | 0.3525 | 0.7265 | 0.5945 | 0.0005 | −0.3975 | −0.0505 | −0.1735 | 0.5985 | −1.1425 | −0.0925 | −0.4025 |
| HRASLS | −0.7255 | 0.0865 | 0.0325 | −0.3605 | 0.1945 | −0.4435 | −0.1145 | 0.1575 | 0.1545 | −0.6855 | −0.5055 | −0.4165 | 0.7015 | 0.3495 | 0.0815 |
| HRB | 0.982 | 1.562 | 0.881 | 0.565 | 2.764 | 3.596 | 1.45 | −0.643 | 0.208 | 0.232 | 1.364 | 4.31 | 1.874 | 2.926 | 0.194 |
| HRSP12 | −0.334 | −1.013 | −0.094 | 0.547 | −0.71 | 0.311 | −0.114 | 0.777 | 0.489 | 0.444 | 0.311 | 1.225 | 1.058 | 0.288 | 0.888 |
| HSD17B2 | 0.2395 | −0.1355 | −0.2765 | 0.6345 | −0.3525 | 0.0785 | 0.7455 | −0.2045 | 0.3645 | 0.4895 | 0.3155 | 0.3865 | 0.1115 | 1.2305 | 2.2535 |
| HSF1 | 0.5425 | −1.3215 | −0.4585 | −0.4965 | 0.3985 | −1.0165 | −0.4345 | −0.6425 | −0.1695 | −0.7925 | 0.3855 | 1.0105 | 0.3785 | −0.6365 | 0.0795 |
| HSPA14 | 2.006 | 0.549 | 1.584 | 0.332 | 2.123 | 1.598 | 1.297 | 0.578 | 0.432 | 0.03 | −0.226 | −0.079 | 1.442 | 0.889 | 0.162 |
| HSPA2 | 0.6345 | −0.4195 | 0.0215 | −0.2635 | −0.4265 | −0.1465 | −0.0935 | −0.2425 | −0.1645 | −1.2125 | −0.4585 | −0.4925 | −0.6045 | −0.2495 | 0.0375 |
| HSPA5BP [symbol] | −0.509 | 0.406 | 0.553 | 0.768 | 1.907 | 0.485 | −0.326 | −0.359 | 0.179 | −0.278 | 0.465 | 0.151 | −0.362 | 0.515 | 0.379 |
| HSPC016 | −0.878 | 0.248 | 0.186 | −0.057 | 0.149 | 0.228 | 0.014 | −0.043 | 0.249 | −0.463 | 0.386 | 0.394 | −0.112 | 0.208 | 0.527 |
| HSPC138 | 0.28 | 1.19 | 0.434 | 0.461 | 0.464 | −0.649 | −0.248 | −0.382 | −0.339 | −0.515 | 0.312 | 0.81 | 0.095 | 0.346 | 0.891 |
| HSPC148 | −0.7535 | 1.3355 | 0.7275 | 0.6985 | 1.7415 | 0.2155 | 0.3685 | −0.0755 | 0.0015 | 0.6925 | −0.0445 | −0.2115 | 0.0235 | 0.9635 | −0.0015 |
| HSPC150 | 0.4795 | −0.0275 | −0.3305 | −0.2315 | 0.3765 | −0.1425 | 0.5155 | −0.1115 | −0.1655 | −0.1585 | −0.4955 | 0.1615 | 0.2025 | −0.3595 | 0.8355 |
| HSPC163 | −1.067 | 0.373 | −0.116 | −0.618 | −0.827 | −0.622 | −0.784 | −0.207 | 0.081 | −0.079 | −0.042 | 0.966 | 1.132 | 1.452 | 2.279 |
| HSPC266 | 0.338 | 0.17 | 0.291 | 0.834 | 0.934 | 0.207 | 0.396 | −0.229 | −0.883 | 0.519 | 0.395 | −0.389 | 0.381 | 0.775 | 1.084 |
| HSU79274 | 0.085 | 0.083 | 0.985 | 0.585 | 0.341 | 0.349 | 0.458 | 0.151 | −0.102 | 0.008 | 0.267 | 1.13 | 0.042 | 0.362 | 0.488 |
| HTATIP2 | 0.028 | 2.19 | −0.308 | 0.006 | −0.459 | 0.381 | 1.11 | −0.25 | 0.118 | −0.25 | −0.539 | −0.481 | −1.211 | −0.125 | −0.432 |
| ICMT | −2.139 | −1.253 | −0.231 | 0.318 | −1.643 | −0.647 | 0.044 | 0.088 | 1.085 | −1.582 | 1.521 | 2.884 | 1.875 | 2.31 | 0.162 |
| ICT1 | 1.128 | 0.96 | 1.354 | 0.68 | −0.044 | 0.897 | 3.035 | 1.483 | −0.075 | 0.992 | 0.313 | 0.516 | −0.12 | 0.247 | 0.846 |
| ID4 | 0.0385 | 0.1195 | −0.4475 | −0.0985 | −0.3975 | 0.0875 | 0.4015 | 0.4365 | −0.9015 | −0.7665 | −0.9805 | −0.0245 | −0.7885 | −1.0015 | −0.3725 |
| IDH2 | 1.783 | −0.038 | 0.366 | 0.487 | 0.858 | 1.152 | 1.875 | 0.721 | −0.1 | 0.801 | −0.068 | 0.837 | 0.511 | −1.781 | −0.492 |
| IDS | 0.2065 | 0.1255 | 1.1995 | −0.4275 | −0.8475 | 0.0015 | 0.0675 | 1.4665 | −0.4505 | −0.6115 | 0.4205 | 0.2475 | 0.8075 | 0.1785 | 0.0455 |
| IFI30 | −0.6355 | −0.2345 | 0.3035 | −0.4535 | 0.3865 | 0.1335 | −0.1015 | 0.4885 | −0.6965 | −0.3085 | −0.0065 | −0.6115 | −0.1385 | 0.4135 | −0.1285 |
| IFI44 | −1.0105 | −0.0975 | 0.6385 | −0.6825 | 0.3695 | −0.1145 | −0.4195 | 0.5275 | −0.0545 | −0.3835 | 0.8865 | −0.1835 | 0.4865 | −0.8975 | −0.5785 |
| IFIT5 | −0.4805 | 0.3065 | −0.6815 | −0.2615 | −0.5915 | 0.0585 | −0.0845 | −0.0975 | −0.0985 | −0.4235 | −0.4705 | −1.0465 | −0.8125 | −0.4575 | −0.3965 |
| IFITM1 | −0.6705 | −0.8225 | −0.0415 | −0.7705 | −0.2995 | 0.1755 | −0.3315 | −0.0475 | −0.0995 | −0.1295 | −0.8515 | −0.0745 | −0.4875 | −0.3905 | −0.1575 |
| IGBP1 | −2.0795 | −1.5925 | −0.9475 | −2.8775 | −0.1335 | −1.9675 | −0.7535 | 0.7545 | 0.9295 | 0.3255 | −2.2925 | −1.1285 | −1.6495 | −1.7075 | −2.2705 |
| IGFALS | 0.4875 | 1.1865 | 0.0395 | −0.3075 | 2.2825 | 2.2955 | 2.5295 | 1.3315 | −0.2015 | −0.2125 | 0.2275 | 0.4875 | 0.0905 | −0.4585 | −0.4225 |
| IGPBP2 | 0.3705 | 0.7555 | −0.0915 | −0.0005 | 1.6135 | 2.1705 | 2.6525 | 1.3385 | −0.0885 | 0.0555 | −0.2485 | 0.1145 | −0.1395 | −0.2485 | −0.2695 |
| IGHG1 | 0.5335 | −0.8435 | −1.6215 | −0.4895 | 1.9135 | 3.9695 | 1.9275 | 0.4585 | 1.9055 | −1.0715 | −0.8845 | −2.1875 | −0.3015 | −1.6325 | −1.9315 |
| IGHG1 | −0.453 | −0.21 | −0.369 | −1.426 | 3.139 | 3.131 | 3.203 | 0.859 | 1.984 | −0.746 | 0.432 | −0.647 | −1.116 | −1.122 | −1.257 |
| IGJ | 0.017 | 0.88 | −0.425 | −1.02 | 1.509 | 2.315 | 1.859 | 0.069 | −0.873 | −0.399 | −0.848 | 0.047 | −1.154 | −1.692 | −1.758 |
| IGKC | −0.811 | −0.373 | −0.551 | −0.714 | 1.713 | 2.732 | 2.12 | 0.322 | −0.358 | −0.29 | −0.801 | −0.078 | −0.261 | −0.594 | −0.456 |
| IGKC | 0.561 | 0.033 | −0.686 | −1.036 | 1.324 | 4.315 | 3.788 | 0.316 | −0.27 | −0.036 | 0.583 | −0.658 | −0.286 | −1.233 | −1.487 |
| IGLC2 | 1.3485 | 0.2005 | 1.2485 | 0.5295 | 0.3965 | 0.3535 | 1.8375 | −0.0835 | −0.2915 | 0.1595 | −0.6495 | −0.7045 | −0.5575 | −1.2085 | −0.7925 |
| IGLL1 | 0.1785 | 0.1835 | 0.1485 | 1.0445 | −0.1825 | −0.3385 | 0.1745 | 0.2255 | −0.0895 | 0.0405 | 0.8045 | 4.6325 | −0.1485 | −0.1865 | 0.2195 |
| IL13RA1 | 0.025 | −0.2 | −0.405 | −0.411 | −0.074 | 0.386 | 0.139 | 0.871 | 0.245 | −0.007 | −0.3 | 1.726 | 0.037 | −0.209 | 1.654 |
| IL17B | −0.8185 | −1.4105 | −0.6335 | −1.1695 | −1.0495 | −0.0935 | −0.6585 | 0.3315 | −0.1575 | −0.1895 | −1.6455 | −1.4415 | −1.0005 | −1.9295 | −1.2345 |
| IL23A | −0.2925 | 0.2335 | −0.1335 | 0.0045 | −0.0345 | −0.0505 | −0.0845 | −0.2345 | 0.1775 | 0.5825 | 0.4445 | 0.3655 | 0.4255 | 0.9015 | 0.5095 |
| IL6ST | 1.3265 | −0.4105 | 0.6435 | 0.8985 | −1.0025 | −1.1295 | −0.2735 | 0.3915 | 0.5545 | 0.7035 | 2.7245 | 0.3125 | 1.2465 | 1.0855 | 1.4485 |
| ILF2 | 0.291 | −0.34 | −0.21 | 0.402 | 0.77 | −0.069 | −0.345 | −0.75 | −0.359 | −0.356 | −0.413 | −0.505 | −0.791 | −0.37 | 0.216 |
| IMPA2 | 0.4045 | −0.0805 | 0.7685 | 1.1005 | −2.2795 | 0.4895 | 2.1145 | −0.8805 | −0.4895 | 0.8825 | 1.8845 | 0.0685 | 0.4385 | 0.4995 | 3.0795 |
| ING1L | −1.364 | −1.086 | 0.211 | −0.531 | 1.244 | −0.403 | 0.858 | 0.114 | 0.058 | −0.023 | −1.878 | −1.459 | −1.689 | −2.772 | −1.896 |
| INHBB | 0.1675 | −0.1265 | −0.5025 | 0.0015 | −0.3025 | 0.3076 | −0.0455 | 0.2215 | 0.2545 | 0.1075 | 0.2325 | 0.6465 | −0.3335 | −0.1115 | 0.1015 |
| INPP4B | −1.3015 | −0.3865 | 0.5595 | 0.2935 | −1.3745 | −0.0985 | −0.0585 | −0.0035 | 0.9155 | 1.7055 | −0.2965 | 0.5475 | 0.5365 | 0.9465 | −0.1185 |
| INSM1 | −0.764 | −0.256 | 0.551 | 0.012 | −0.229 | 0.138 | 0.756 | −0.439 | 0.474 | 0.838 | −0.731 | −0.077 | 0.128 | −0.325 | 0.222 |
| IQCA | 0.461 | −1.248 | −0.418 | −0.448 | −0.148 | 0.307 | −0.076 | −0.017 | 0.347 | −0.452 | −2.536 | −1.344 | −1.484 | −1.787 | −2.472 |
| IQCE | 0.446 | 0.198 | 0.256 | 0.002 | 0.951 | 2.298 | 1.137 | 0.991 | 0.29 | 0.712 | 1.021 | 0.198 | 0.727 | −0.353 | 0.265 |
| IQGAP2 | 0.1135 | −0.0425 | −0.1975 | −0.3325 | 2.1845 | 2.6245 | 2.5875 | 2.2115 | −0.0975 | −0.3825 | 0.8895 | 0.3875 | 0.9435 | −0.2895 | −0.6045 |
| IRF4 | −2.0175 | 1.1145 | −0.2955 | 2.1855 | −0.5405 | 0.6025 | −2.6615 | 0.6805 | −0.6545 | −1.2775 | −1.8435 | 2.7385 | −0.7295 | 2.8515 | 3.0585 |
| IRTA2 | 0.484 | 0.473 | −0.165 | 0.219 | 0.873 | 1.122 | 0.625 | 1.588 | −0.792 | 0.323 | 0.249 | 0.039 | 0.491 | −0.884 | 2.44 |
| IRX4 | 0.0515 | −0.3045 | 0.3815 | −0.1515 | 0.6025 | 0.3415 | −0.0195 | 0.8185 | −0.1865 | −0.1735 | 0.7295 | −0.2475 | 0.8895 | −0.2545 | 0.3525 |
| ISG20 | −0.449 | 0.551 | 4.497 | 1.548 | −0.234 | −0.239 | 0.473 | −0.526 | −0.995 | 0.522 | 0.837 | −0.22 | −1.205 | −2.303 | 0.45 |
| ISGF3 [symbol] | −2.039 | −1.143 | −0.953 | −0.763 | −0.106 | −0.573 | −1.188 | 1.908 | −0.801 | −1.396 | −2.538 | −1.218 | −1.936 | −2.062 | −0.542 |
| ITGB4 | −0.335 | −0.285 | −0.442 | −0.631 | 0.057 | −0.248 | 0.391 | 0.623 | 0.011 | 1.477 | 0.839 | 2.818 | 0.64 | 0.495 | 2.023 |
| ITPR1 | −2.875 | 0.705 | −0.838 | −3.828 | 0.53 | −0.108 | −2.394 | 0.157 | 0.183 | −0.006 | −0.28 | −2.491 | 0.101 | −1.132 | −2.445 |
| JARID1A | −0.169 | 0.557 | −0.154 | 0.144 | 0.01 | 0.058 | −0.417 | −0.179 | 0.318 | 0.983 | 0.206 | 0.965 | −0.272 | 0.577 | 0.153 |
| KARCA1 | 0.283 | 0.218 | 0.16 | 0.129 | 0.232 | 0.274 | −0.444 | −0.034 | 0.092 | 0.123 | 0.216 | −0.32 | 0.162 | 0.202 | −0.395 |
| KATNA1 | −0.4135 | 0.9855 | 0.1085 | 0.2985 | −0.3805 | −0.8785 | 0.4525 | 0.0455 | 0.2085 | 0.0805 | −0.0205 | −0.0695 | 0.2885 | 0.1145 | 0.0225 |
| KCNC2 | 0.117 | −0.345 | −0.318 | −0.802 | 0.323 | 0.836 | 0.912 | −0.209 | 0.026 | −0.198 | −0.295 | −0.357 | 0.149 | −0.252 | −0.245 |
| KCND2 | −0.066 | 0.622 | −0.625 | 0.338 | −1.051 | 0.094 | 1.692 | 0.669 | 0.207 | −0.542 | 0.945 | 2.988 | 1.849 | 2.583 | 0.335 |
| KCNK4 | −0.031 | 0.01 | 0.404 | 1.911 | 1.132 | 0.708 | 1.974 | −0.511 | −0.436 | −0.349 | −0.301 | −0.937 | −0.577 | 1.505 | −1.425 |
| KCNN4 | −0.1335 | −0.5885 | −0.1215 | −0.6275 | −0.8805 | −1.0185 | 0.6885 | 0.3625 | −0.0885 | 0.3755 | 0.3265 | 1.3435 | 1.6675 | −0.4495 | 1.8065 |
| KCNS3 | −1.0615 | 0.5945 | −0.0125 | −1.4505 | −0.9625 | −0.5325 | −0.1685 | 0.0445 | 0.0895 | 0.4195 | −1.3815 | −0.7355 | −0.3505 | −1.2375 | −0.7375 |
| KCTD15 | −0.132 | 0.426 | 1.277 | 0.639 | −0.661 | 0.843 | 0.807 | −0.5 | 0.027 | 0.822 | 0.101 | −0.893 | 0.409 | 0.612 | −0.264 |
| KCTD3 | −0.111 | 0.824 | 0.011 | −0.154 | −0.054 | −0.125 | −0.324 | 0.256 | −0.04 | −0.35 | 0.736 | −0.054 | 0.646 | 1.545 | 0.765 |
| KDELR2 | 1.044 | 1.153 | 0.358 | 0.636 | 0.577 | −0.278 | −0.688 | −0.587 | −0.271 | 0.276 | −0.32 | −0.981 | −1.137 | 0.002 | 0.887 |
| KIAA0020 | 0.674 | 0.858 | −0.102 | −0.138 | −0.701 | 0.005 | −0.569 | 0.459 | 0.113 | −0.041 | 0.148 | 0.881 | 0.328 | 0.148 | 0.322 |
| KIAA0101 | 1.0915 | 0.8765 | 0.2915 | 0.5085 | 1.4355 | 0.1525 | 1.1755 | 0.2895 | 0.9485 | −0.4405 | −0.0075 | 0.1655 | −0.9665 | −0.3575 | −1.3235 |
| KIAA0153 | −0.9195 | 0.6125 | 0.2425 | 0.1505 | 0.4485 | 0.3575 | −0.7815 | −0.1065 | 0.0955 | −0.0015 | −0.5045 | 0.1218 | 0.0665 | −0.8315 | −0.6145 |
| KIAA0182 | 0.7025 | 0.7645 | 0.6585 | 0.9715 | 0.3245 | 0.2155 | 1.1715 | −0.0665 | 0.5315 | 0.2275 | 0.4455 | 0.2335 | −0.3075 | 0.8795 | 0.5455 |
| KIAA0196 | 0.1645 | −0.4375 | −0.0655 | −0.2825 | 0.2515 | 0.3495 | 0.1865 | 0.1065 | 0.9335 | −0.1365 | −0.5185 | 0.1045 | −0.0245 | −0.8485 | −0.3995 |
| KIAA0241 | 0.152 | −0.16 | 0.068 | 0.175 | 0.025 | 0.506 | −0.292 | 0.104 | −0.023 | 0.184 | −0.064 | −0.008 | −0.653 | 0.184 | 0.431 |
| KIAA0251 | −0.0635 | −0.2345 | 0.0715 | 0.0175 | 0.3425 | −0.1026 | 0.6585 | 0.3085 | −0.8365 | −0.3876 | 0.3535 | 0.2525 | 0.1395 | −0.0345 | 0.0745 |
| KIAA0274 | −2.89 | −0.836 | −0.692 | −1.113 | −0.818 | −0.11 | −0.544 | 0.564 | −0.348 | −0.716 | −2.925 | 0.43 | 0.041 | −0.727 | −0.002 |
| KIAA0515 | −0.4715 | −0.4445 | 0.0205 | 0.3675 | 0.5025 | 0.5435 | 0.7585 | 1.2835 | −0.0205 | −0.8985 | 0.9125 | 0.0415 | 0.1525 | 0.8775 | 0.2815 |
| KIAA0582 | −0.5785 | 0.2455 | −0.1845 | −0.9445 | 0.2485 | 0.3715 | 1.7785 | −0.0285 | −0.3765 | 0.4905 | −0.0035 | 0.3005 | −0.3885 | 1.1765 | −0.9115 |
| KIAA0650 | −0.2815 | 0.0645 | 1.1665 | 0.6065 | −0.3845 | 0.6595 | 0.7925 | −0.4255 | 0.1145 | −0.0265 | 0.5925 | 1.3995 | −0.2015 | −0.4585 | 1.1335 |
| KIAA0703 | −0.751 | −0.684 | 0.002 | −1.199 | −0.89 | 0.478 | 0.111 | 0.326 | 0.987 | 1.11 | −0.188 | 0.601 | 0.489 | 0.071 | 1.147 |
| KIAA0830 | 1.0925 | −1.8265 | −1.0765 | −0.3535 | −0.4575 | −0.9995 | −0.0545 | 0.1755 | 0.0195 | −0.1575 | −2.8265 | −0.8985 | −0.2925 | −1.3405 | −2.8245 |
| KIAA0980 | 1.3475 | −0.9895 | −0.0165 | 0.5385 | 0.2535 | 0.1965 | 1.2005 | −0.8595 | −0.2785 | 0.4965 | −1.0655 | −1.1735 | −0.0915 | −0.7805 | −0.4275 |
| KIAA1324 | 0.342 | −0.204 | −0.299 | −0.157 | −0.39 | −0.166 | 0.809 | 0.489 | 0.345 | −0.318 | −0.788 | −0.606 | −0.294 | 0.068 | −0.15 |
| KIAA1363 | −0.003 | −1.14 | −0.523 | −1.385 | 0.548 | −0.125 | −0.521 | −0.438 | −1.008 | −1.108 | −1.615 | −1.146 | −1.387 | −0.898 | −0.634 |
| KIAA1683 | 0.1165 | 0.5965 | 0.2155 | −0.0955 | 0.8615 | −1.4445 | −0.4405 | 0.0695 | −0.0665 | −0.2515 | 0.6065 | −0.5055 | −0.1385 | 0.5985 | 0.8985 |
| KIF12B | −0.7905 | −0.2315 | 0.2895 | 0.3605 | −0.4805 | −0.4285 | 0.5405 | −0.1075 | 0.2545 | 0.8455 | 0.2155 | 0.8545 | 0.1695 | 1.1715 | 1.7345 |
| KIF24 | −0.0905 | 0.1505 | −0.0695 | −0.2865 | 0.2825 | 0.4425 | −0.1585 | 0.3446 | 0.2895 | 0.0605 | −0.1155 | 0.1385 | 0.0785 | −0.2045 | −0.3685 |
| KIN | −2.3375 | −1.1305 | −0.6445 | −1.4175 | −1.7205 | −0.8375 | 0.0265 | 0.2595 | 0.9255 | 1.2005 | 0.1135 | 0.4955 | 2.1445 | 3.1315 | 3.9855 |
| KIRREL3 | −0.5005 | −0.6545 | −0.4495 | 2.4355 | −0.5995 | −0.2175 | 1.0415 | 0.3125 | 0.7775 | −0.7755 | 1.4965 | 2.5425 | −0.4555 | 2.2755 | −0.4305 |
| KIT | −0.5505 | −0.2825 | −0.4755 | 5.2335 | −1.1995 | −0.9515 | 1.1045 | −0.6705 | 1.4816 | −1.3335 | 1.5805 | 3.3525 | −1.5145 | 4.6975 | 0.8325 |
| KLK5 | −0.295 | −0.293 | −0.288 | 0.213 | −0.573 | −1.019 | 1.703 | 0.603 | 0.757 | −0.648 | 0.967 | 3.593 | −0.558 | 2.828 | 0.22 |
| KLK6 | −1.459 | −0.548 | −0.503 | 3.787 | −0.897 | −0.789 | 0.241 | −0.297 | 0.088 | −0.278 | 1.491 | 2.727 | 0.162 | 2.758 | −0.394 |
| KLK7 | 0.078 | 0.294 | −0.076 | 1.652 | 0.86 | −0.24 | 0.015 | 0.447 | −0.341 | −0.269 | 1.55 | 0.557 | 1.232 | 1.854 | 1.059 |
| KLK8 | 1.1525 | 0.6655 | 0.8235 | 0.2005 | 0.6035 | 0.0425 | 1.5315 | 0.0025 | −0.1745 | 0.7925 | 0.4825 | −0.0025 | −0.2115 | 0.7915 | 0.6535 |
| KNTC2 | 0.15 | 2.049 | 3.055 | 0.843 | 0.561 | 0.594 | 1.372 | −1.089 | 0.634 | 0.637 | 1.08 | −0.465 | −0.212 | 1.3 | 2.048 |
| KPNA2 | −0.1635 | −0.5075 | 0.0375 | −0.0655 | −0.4125 | 2.2135 | −0.0725 | −0.3835 | −0.3865 | 1.9885 | −0.2825 | −0.3375 | −0.5425 | −0.8205 | −0.1565 |
| KREMEN2 | 0.0745 | −1.0195 | −0.3455 | 0.4545 | −1.0305 | 1.2985 | 0.5035 | 0.8045 | 1.0056 | 0.7365 | 1.4075 | 3.1975 | −1.4105 | −0.0745 | −0.3185 |
| KRT10 | 0.2855 | −1.3885 | −0.4535 | 0.0355 | −1.5685 | 1.3475 | 0.6025 | 0.5775 | 2.3085 | 0.8265 | 1.9485 | 3.2955 | −0.9035 | −0.1915 | −0.9345 |
| KRT13 | −0.854 | 0.305 | 0.79 | 1.274 | −1.455 | −1.415 | 1 | 0.681 | −0.89 | −1.392 | 4.039 | 3.938 | −1.357 | 2.29 | 2.203 |
| KRT15 | 0.405 | −0.12 | −0.218 | 0.355 | 0.286 | 1.935 | −0.049 | −0.12 | 0.312 | −0.249 | −0.542 | −0.267 | −1.296 | 0.466 | −1.273 |
| KRT17 | −1.5615 | 1.0525 | −0.4335 | −1.1875 | −2.1235 | 3.1265 | 4.2695 | 0.6325 | 0.3815 | 4.5035 | 2.1735 | 4.1285 | 1.4425 | 1.8065 | −1.6325 |
| KRT18 | −2.174 | −0.858 | 0.334 | 2.024 | −2.602 | −3.098 | 1.536 | 1.845 | −1.187 | −2.511 | 0.523 | 4.02 | 0.215 | 1.855 | 3.309 |
| KRT23 | 0.1595 | −0.0645 | 0.0085 | −0.0485 | 0.0015 | −0.4835 | −0.3345 | −0.0385 | −0.0465 | −0.1425 | 0.2895 | −0.2745 | 0.1485 | −0.1015 | 0.0585 |
| KRT6B | −0.123 | −0.244 | −0.124 | −0.158 | −0.08 | 0.089 | 0.211 | −0.087 | 0.359 | 0.21 | −0.095 | −0.637 | −0.018 | −0.08 | 0.373 |
| KRTAP3-3 | −1.1045 | −1.0765 | −0.9255 | 0.2125 | −1.3315 | −1.0005 | −2.3315 | 0.1545 | −0.2245 | −0.9495 | −1.1715 | 0.9675 | −1.7395 | −1.5895 | −1.2495 |
| KRTHA7 | −1.4055 | −0.5355 | 0.2545 | 1.2345 | −1.7655 | −2.9055 | 0.1045 | −0.1855 | −0.0425 | 0.2845 | 0.9045 | 3.7845 | −0.8475 | −1.6665 | 1.4475 |
| LAMA3 | −0.123 | 0.273 | −0.171 | −0.416 | 0.543 | −0.424 | −0.743 | −0.123 | 0.135 | 0.184 | −0.233 | −0.476 | 0.429 | −0.938 | −0.422 |
| LAMB3 | 0.512 | 0.494 | −0.218 | −0.295 | 0.488 | 1.844 | 2.164 | 1.148 | −0.12 | −0.145 | −0.011 | −0.483 | −0.134 | −0.242 | −0.481 |
| LASS2 | 0.2795 | 0.0025 | 0.3505 | 0.9425 | 0.2795 | 0.4435 | −0.5985 | 0.5385 | −0.2825 | −0.5555 | 0.7345 | 0.5685 | 0.1795 | 0.0265 | 0.4235 |
| LAX | −0.4945 | −0.7915 | 1.0295 | 0.3475 | 0.8135 | −0.7575 | 1.1145 | 1.5795 | 0.8235 | −1.6035 | 1.3675 | 0.1165 | −1.6825 | 0.8535 | 1.9295 |
| LBR | −0.8035 | 1.0975 | −0.1105 | −1.3975 | 0.3195 | 0.0575 | −0.6825 | 0.2665 | 0.0975 | −1.2155 | −0.1145 | 1.8345 | 2.8125 | 2.7095 | 1.6395 |
| LCE1B | −1.5635 | 0.0045 | −0.1585 | −0.4615 | 0.0175 | 0.1535 | −0.4095 | −0.1845 | −0.7415 | −0.9695 | −0.9535 | 0.1065 | −1.0985 | −0.4965 | −0.0155 |
| LDHB | −0.64 | 0.369 | −0.441 | −0.563 | −0.361 | −0.073 | −0.78 | 0.272 | −0.003 | −0.374 | −0.068 | 0.052 | −0.213 | −0.385 | −0.326 |
| LDOC1L | −0.268 | −1.159 | 0.013 | 0.451 | 1.356 | −1.143 | 0.195 | −0.316 | −0.704 | −1.001 | −1.425 | −0.333 | −0.894 | −1.173 | −1.723 |
| LETMD1 | 0.0335 | 0.3435 | −0.3725 | −0.8095 | 0.7545 | 1.6595 | −0.9375 | 2.9945 | 1.2535 | 0.5375 | −0.2055 | 0.9435 | 0.9595 | −0.1955 | −0.8445 |
| LFNG | −0.673 | 0.629 | −0.321 | −0.628 | −0.807 | −0.556 | −0.427 | 0.094 | 0.1 | 0.761 | −0.183 | 0.455 | 0.422 | −0.106 | −0.177 |
| LGALS2 | −0.314 | −0.276 | −0.954 | −0.049 | 0.096 | −0.156 | 0.165 | −0.7 | −0.398 | −0.609 | −0.717 | −1.094 | −1.553 | −0.624 | −0.969 |
| LGTN | −1.309 | −1.755 | −0.043 | −1.545 | −0.639 | −1.423 | −0.49 | 0.136 | 0.365 | 0.768 | −0.589 | 0.861 | 0.393 | −1.194 | −1.13 |
| LNX | −1.5185 | −0.8295 | −0.8525 | −1.2535 | 0.5955 | −0.4945 | 0.5835 | −0.8065 | −0.7135 | −1.0845 | −1.1155 | −0.2075 | 0.6525 | −0.3785 | 0.0175 |
| LOC11247 | 0.275 | −0.005 | −0.248 | −0.237 | 0.243 | 0.114 | −0.977 | −0.135 | −0.096 | 0.092 | −0.382 | −1.056 | −0.687 | 0.671 | −1.245 |
| LOC11492 | −0.541 | −0.442 | 0.357 | 0.181 | −0.191 | −0.57 | 0.147 | 0.308 | −0.277 | −0.271 | −0.958 | −1.259 | −1.264 | 0.695 | −0.185 |
| LOC11497 | 0.0735 | −0.3145 | −0.2085 | −0.1825 | −0.0085 | −0.1885 | 0.3205 | 0.1225 | −0.1845 | −0.5595 | 0.1395 | −0.0935 | −0.2165 | −0.2755 | −0.4195 |
| LOC11621 | 2.6265 | 2.6015 | 2.8805 | 1.9075 | 2.7865 | 2.6845 | 2.7675 | −0.8565 | −0.9595 | 1.8445 | −0.3815 | −1.7465 | −0.6835 | −0.9475 | −1.8225 |
| LOC11644 | 0.1345 | −0.0075 | 0.0905 | −0.8805 | −0.9505 | −0.4485 | −1.2535 | −1.2705 | 0.1875 | −0.7355 | −2.6935 | −2.9725 | −1.7585 | −2.9455 | −2.5515 |
| LOC12022 | 0.1625 | 0.0255 | −0.2755 | −0.3145 | −0.0515 | −0.1655 | 0.0065 | 0.2225 | 0.4895 | 0.1305 | −0.0445 | 0.2615 | 0.0545 | −0.8945 | −0.2805 |
| LOC12422 | 1.0485 | −0.6585 | −0.1805 | −0.3555 | −0.4785 | −0.4455 | 0.5805 | 0.9785 | 1.2195 | 0.4155 | 0.0435 | −0.2565 | −0.1015 | −0.0345 | −0.1065 |
| LOC14654 | −0.531 | −0.119 | −0.479 | 0.088 | 1.124 | −0.21 | −1.077 | −0.641 | −0.05 | −0.08 | −2.168 | −1.492 | −1.299 | −2.918 | −1.552 |
| LOC15506 | −0.102 | −0.519 | −0.247 | −0.82 | −0.631 | 0.234 | −0.81 | −1.03 | 1.024 | −0.219 | −0.474 | 0.198 | 0.634 | −0.173 | 0.188 |
| LOC20189 | −0.2795 | −1.0105 | 0.3745 | 0.4015 | −0.4935 | 1.6585 | 1.5395 | −0.4945 | 2.4935 | −0.9815 | −1.0485 | −0.9855 | 1.6055 | 3.8855 | −0.6155 |
| LOC25398 | 0.553 | 0.204 | 0.437 | −0.002 | 0.529 | −0.55 | 0.673 | 0.418 | 0.27 | −0.287 | 0.429 | 0.432 | 0.892 | 0.188 | 0.158 |
| LOC33990 | 0.708 | −0.08 | −0.392 | −0.855 | −0.819 | −0.432 | 1.858 | 1.116 | −0.088 | 0.149 | −1.94 | 0.079 | −0.882 | −1.685 | −1.355 |
| LOC34817 | −1.0585 | −0.4105 | 0.3245 | −0.7105 | 0.1145 | −0.4145 | 1.2405 | −0.2115 | 0.3015 | −0.9365 | 0.8265 | 0.9685 | 1.7585 | 0.2585 | 0.1355 |
| LOC40045 | −0.302 | 0.679 | 1.949 | 2.153 | 0.645 | −0.204 | 0.155 | −0.38 | −0.2 | 0.223 | −0.304 | −0.894 | −0.71 | 0.96 | −0.81 |
| LOC44089 | −0.415 | −0.01 | 0.596 | 0.935 | 0.66 | 0.517 | −0.367 | −0.131 | 0.145 | −0.056 | 0.248 | 0.055 | 0.301 | −0.268 | −0.182 |
| LOC49386 | −1.002 | −1.047 | −0.335 | −0.592 | −1.201 | −0.95 | −0.291 | −0.498 | 0.003 | 0.497 | −0.791 | 0.057 | −0.687 | −0.454 | −0.89 |
| LOC51123 | 0.387 | 0.516 | −0.155 | −0.385 | 0.306 | 0.332 | 0.023 | 0.32 | −0.139 | 0.502 | −0.472 | −0.307 | −0.766 | −0.895 | −0.018 |
| LOC51161 | 0.1225 | 0.2605 | 0.5825 | 0.0845 | 0.9835 | −0.0645 | 0.8855 | 0.0275 | 0.6805 | 1.2785 | −1.0355 | −1.1825 | −0.9765 | −0.5225 | −1.7305 |
| LOC51321 | −0.6585 | −0.5635 | −0.9915 | 0.0125 | 0.3425 | 1.0155 | −0.7675 | 0.4465 | 0.0695 | −1.0835 | −0.6705 | −0.4045 | −0.1795 | −0.5505 | −0.4655 |
| LOC51760 | 0.4505 | 0.4935 | −0.2855 | 2.4385 | 0.1765 | 0.0975 | −1.0585 | −0.8545 | −0.1575 | 0.5135 | 1.2715 | 1.1995 | 0.4385 | 2.4715 | 0.1035 |
| LOC81558 | −2.071 | −2.05 | −0.931 | −0.372 | 0.428 | −1.692 | −3.262 | −1.013 | 1.119 | −0.883 | −1.39 | −1.453 | −0.95 | −0.811 | −1.057 |
| LOC81569 | −0.2085 | −0.0055 | 0.3745 | −0.5785 | 0.8555 | 1.3465 | 0.4225 | −0.1475 | 0.3845 | 0.4805 | −0.0285 | 0.5095 | −0.7405 | −1.9405 | 0.4375 |
| LOC90355 | 0.008 | 0.64 | −0.768 | −0.195 | 0.309 | −0.682 | 0.597 | −0.649 | 0.023 | −0.548 | −0.481 | −1.07 | 0.629 | −0.215 | −0.265 |
| LOC90701 | −0.032 | −0.095 | −1.896 | 0.938 | 0.768 | 1.331 | 1.085 | 1.765 | 0.427 | −1.714 | −1.374 | −0.109 | −1.158 | 0.026 | 0.022 |
| LONP | −0.4505 | −0.1795 | −0.0535 | 0.0935 | 0.9795 | 0.1575 | −0.0785 | 0.0025 | −0.4715 | −0.8065 | −0.6935 | −0.0365 | −1.4625 | −0.4475 | −0.5755 |
| LRAP | −0.4785 | −0.9045 | −0.2095 | −0.0935 | −0.5966 | −0.3655 | 0.2075 | −0.2515 | 0.3325 | −0.4055 | −1.9975 | −0.4155 | −1.2375 | −1.2325 | −0.2155 |
| LRBA | −1.5095 | −0.9305 | −1.2755 | −1.5025 | −1.0525 | −0.6585 | 0.0725 | −0.1185 | 0.2155 | 0.0415 | −0.8185 | 1.4275 | 1.1855 | 1.0115 | 2.0635 |
| LRIG1 | 0.0545 | 1.1495 | 1.2455 | −0.4265 | −0.8875 | 1.8825 | 0.9135 | 0.7715 | 0.2405 | −0.4085 | 0.0505 | 0.8295 | 1.1125 | 2.2415 | 1.6535 |
| LRP6 | −0.854 | 0.545 | 0.318 | −0.124 | −0.438 | −0.622 | −0.292 | 1.271 | 0.967 | 0.143 | −0.967 | −0.895 | −0.085 | 0.483 | −1.558 |
| LRP8 | −0.985 | −0.261 | 0.175 | −0.55 | 0.079 | −0.363 | −0.465 | −0.166 | −0.051 | 0.176 | −1.416 | −0.631 | −0.863 | −0.981 | −0.017 |
| LRRC17 | −0.3945 | 0.4205 | 0.1755 | −0.2715 | 0.4905 | −0.0425 | 0.0335 | 1.4145 | −0.1765 | 0.7405 | 0.5625 | −0.3585 | −0.7975 | 0.3215 | 0.1015 |
| LRRC2 | 0.2945 | 0.4845 | −0.1785 | 0.3085 | −0.1155 | 0.0295 | 0.2505 | 0.0685 | −0.0115 | −0.1205 | −0.5205 | −0.2205 | −0.3435 | 0.5465 | 0.9585 |
| LSM1 | 0.8725 | −0.6245 | −0.8785 | −0.7205 | 0.0555 | 1.5645 | 1.6705 | 3.4265 | 0.9745 | 0.1985 | 1.1635 | 3.1105 | 1.6345 | −1.2445 | 0.1705 |
| LSM8 | −0.019 | 0.222 | 0.144 | 0.881 | 0.167 | −0.415 | 1.829 | 0.182 | −0.382 | −0.219 | 1.485 | 2.181 | −0.44 | 3.505 | 3.973 |
| LTB | −0.117 | −0.797 | −0.145 | 1.557 | −0.49 | 1.055 | 1.543 | 1.047 | −0.708 | −0.673 | 0.011 | 0.864 | 2.216 | −0.891 | −0.388 |
| LY6D | −1.105 | 0.275 | 0.415 | 0.125 | 1.535 | −0.085 | −1.035 | −0.495 | −1.027 | −0.535 | −0.175 | −0.435 | −1.484 | 1.266 | 1.971 |
| LYN | −0.791 | 0.156 | −0.032 | −0.192 | 0.301 | −0.049 | 0.28 | −0.126 | −0.345 | −0.834 | 0.548 | 0.532 | 1.394 | 1.188 | −0.551 |
| MA02L1 | 2.718 | 0.668 | 0.833 | 0.377 | 1.321 | −0.072 | 0.761 | −0.33 | −0.296 | 0.372 | 1.269 | 0.2 | 0.092 | 2.716 | 0.003 |
| MADP-1 | 3.9755 | 0.1365 | 0.2525 | 0.2305 | 0.2525 | −0.2205 | 2.1735 | −0.5475 | 0.1435 | 0.1885 | 2.3875 | −0.8285 | 0.9235 | 4.3895 | −0.2305 |
| MAGEA12 | 3.16 | −0.112 | −0.203 | −0.1 | −0.175 | −0.006 | 1.385 | −0.568 | −0.01 | 0.234 | 1.944 | 0.682 | 0.47 | 3.51 | 0.22 |
| MAGEA2 | 3.9705 | 0.8945 | 0.7715 | 1.1645 | 2.2035 | −0.5595 | 1.9875 | −0.6825 | 0.4445 | 0.7425 | 3.1155 | 3.7905 | −0.2125 | 5.3005 | −0.7795 |
| MAGEA3 | 0.0215 | 0.7895 | 0.7385 | 0.0755 | 0.2845 | −0.3315 | −0.5765 | 0.9305 | 0.1785 | 0.0275 | 1.1845 | 0.3875 | −0.4815 | 2.7015 | 1.3195 |
| MAGEA4 | 0.104 | 0.125 | −0.454 | 0.36 | −0.277 | −0.982 | −1.111 | 0.654 | −0.075 | −1.007 | 8.671 | 1.452 | 0.065 | 1.583 | −0.166 |
| MAGED4 | 0.0115 | 0.1635 | 0.3555 | −0.1825 | −0.0135 | 0.0815 | 0.9295 | 0.5315 | 0.9385 | −0.0605 | 0.1385 | 0.0845 | −0.7935 | −0.2115 | −0.8755 |
| MAGI1 | −1.0695 | 0.4565 | 0.9595 | 0.5935 | 0.9155 | 0.8605 | −0.5985 | −0.8855 | −0.2395 | −0.4585 | −1.0415 | −0.4285 | −0.1895 | −1.4495 | −2.8025 |
| MAGI-3 | 0.771 | −0.536 | −0.282 | −0.243 | −0.206 | 0.823 | 1.064 | 0.907 | 0.423 | 0.517 | −0.437 | 0.476 | 0.889 | −0.68 | 1.487 |
| MAL2 | −0.924 | −0.24 | 0.773 | −0.508 | 0.562 | −1.623 | 0.582 | 0.087 | −0.698 | −0.456 | 0.585 | −0.816 | 0.224 | −1.022 | 1.786 |
| MAN2B1 | 0.1585 | −0.9465 | −0.8745 | −0.3675 | 0.9945 | −0.4275 | 3.9315 | 8.1225 | −0.5595 | 0.8175 | 1.3515 | 3.5975 | 0.1025 | 1.1975 | 0.8155 |
| MAPK8IP2 | 0.5555 | 2.6395 | −0.5235 | 0.5605 | 0.7845 | 1.5345 | 0.3995 | 2.2415 | −0.1745 | 0.1065 | 0.4895 | −0.9065 | 0.4855 | −1.0165 | −0.5355 |
| MARCO | −0.144 | −0.982 | 1.33 | 1.43 | 0.848 | 0.276 | −2.178 | −1.437 | 0.154 | −1.117 | −0.564 | −0.605 | −0.068 | −0.415 | −0.599 |
| MARLIN1 | 0.287 | −0.224 | 0.054 | 0.285 | 0.186 | −0.31 | −0.038 | −8.192 | −0.211 | −0.088 | 0.911 | 0.228 | −0.011 | 0.843 | 0.495 |
| MASS1 | 1.6945 | 0.2045 | 8.2245 | −0.4185 | 0.8355 | −0.2445 | 0.6575 | 0.0015 | −0.1505 | −0.1985 | 0.9155 | 0.7925 | −0.3235 | 0.3075 | 0.8025 |
| MCM2 | −1.8195 | −0.2065 | 0.5815 | 0.4135 | −1.3875 | 1.9735 | 1.8215 | −0.2425 | 2.1275 | −0.0195 | 0.5845 | −0.1625 | −0.2125 | 1.8455 | 0.5815 |
| MCM3 | 0.684 | 0.259 | 0.133 | −0.221 | −0.022 | −1.392 | −0.019 | −1.417 | 1.628 | 0.334 | −0.557 | 0.171 | −0.792 | −2.273 | −0.603 |
| MESP1 | −0.4975 | 0.7995 | 1.3745 | 0.7475 | −0.0775 | −0.2005 | 0.8175 | 0.4415 | 0.0275 | −0.0015 | 0.4505 | −0.1625 | 0.7895 | 0.4435 | −0.6775 |
| METRN | 0.2465 | 0.3275 | 8.4385 | 0.3525 | 0.2305 | −0.3485 | −0.3065 | −0.4855 | −0.2255 | −0.1525 | 0.6105 | 1.3495 | 0.6275 | 0.2235 | 0.1635 |
| MFAP2 | 0.461 | 0.44 | 0.337 | 0.679 | −0.166 | 1.175 | 0.427 | 0.928 | −0.079 | 0.269 | 1.399 | 0.621 | 0.308 | 0.15 | −0.111 |
| MFGE8 | 0.3215 | 0.3725 | 0.4955 | −0.1475 | −8.7105 | 0.1775 | 0.4415 | 0.0955 | −0.0625 | 0.2115 | 0.5375 | 0.4075 | −0.0885 | 0.8115 | 0.8885 |
| MGAT4B | 0.182 | −0.149 | 0.385 | −0.317 | 0.435 | 0.67 | 1.343 | 1.81 | 0.196 | −0.001 | 0.486 | 0.412 | 0.734 | −0.372 | −0.276 |
| MGC1091 [symbol] | 0.6405 | 0.9235 | 0.5435 | 1.8125 | 0.2675 | −0.0525 | −0.1385 | 1.0905 | −0.0235 | 0.5175 | −0.6405 | −1.6655 | −0.4115 | −1.2365 | −2.7635 |
| MGC1098 [symbol] | 1.3185 | −1.3425 | −0.9815 | 0.0265 | 8.2755 | −0.5855 | −0.5545 | −0.1025 | −0.0845 | −0.0635 | 0.0405 | 0.3145 | −0.4735 | −0.4155 | 4.5525 |
| MGC1124 [symbol] | −0.505 | −0.326 | 0.369 | 0.002 | −0.2 | 0.185 | −0.087 | 0.082 | 8.202 | −0.363 | 0.478 | 0.47 | −0.17 | 0.393 | 1.129 |
| MGC1127 [symbol] | −0.073 | −0.622 | −0.902 | −0.247 | −0.426 | −0.441 | −0.182 | 0.447 | 8.334 | 1.373 | 0.424 | 3.035 | 0.169 | 1.9 | 2.769 |
| MGC1312 [symbol] | 0.312 | 0.012 | −0.348 | 0.382 | 0.233 | 0.171 | −0.407 | 0.017 | 0.267 | −0.053 | −0.502 | −0.012 | −0.255 | −0.387 | −1.466 |
| MGC1318 [symbol] | −0.225 | −0.315 | 0.223 | −0.028 | 0.057 | −0.18 | −0.055 | 0.359 | −0.255 | 0.154 | −0.431 | 0.015 | −0.537 | −0.367 | −0.182 |
| MGC1414 [symbol] | −0.0775 | 0.4315 | 0.0295 | 0.4485 | −0.1875 | −0.1155 | −0.3035 | −0.0985 | −0.2045 | −0.3235 | 0.0585 | −0.1335 | 0.0555 | 0.3135 | 0.8095 |
| MGG1428 [symbol] | −0.757 | −0.94 | −0.828 | −0.553 | −0.266 | 0.469 | 0.486 | 0.194 | −0.512 | 0.38 | −0.946 | −0.228 | −0.768 | −0.179 | −0.728 |
| MGC1481 [symbol] | −0.105 | −0.43 | 0.171 | −0.224 | −0.04 | 0.524 | 2.37 | 0.572 | 0.066 | −0.495 | 0.764 | 1.618 | 0.182 | 1.196 | 2.813 |
| MGC1580 [symbol] | 0.0635 | 0.1195 | 0.1465 | 0.0775 | 0.4105 | −0.0035 | 0.0275 | 0.5405 | 0.0415 | −0.3575 | 0.0925 | 0.6195 | −0.1645 | 0.2835 | −0.1795 |
| MGC1663 [symbol] | 0.498 | −0.238 | −0.781 | −0.188 | −0.123 | −0.258 | −0.145 | 0.186 | −0.48 | 0.477 | −0.328 | 0.093 | −0.267 | 0.459 | 0.632 |
| MGC1873 [symbol] | −0.071 | 0.932 | 1.703 | 1.072 | 0.193 | −0.338 | 1.644 | −0.203 | −0.268 | −0.046 | 0.852 | 2.488 | 1.642 | 1.989 | 2.295 |
| MGC2053 [symbol] | −1.535 | 0.211 | −0.357 | −0.112 | 0.848 | −1.398 | −1.062 | −0.842 | −0.611 | 0.024 | −1.242 | −0.233 | −1.311 | −0.452 | 0.23 |
| MGC2398 [symbol] | 0.005 | 1.035 | 0.989 | 0.849 | 8.137 | 1.167 | −0.006 | −8.264 | 0.625 | −0.157 | 0.753 | 0.076 | −0.308 | 1.563 | 2.179 |
| MGC2610 | 0.815 | 0.162 | −0.643 | −0.595 | 1.928 | 2.242 | 1.829 | 0.854 | −0.52 | −0.072 | 0.048 | 0.466 | −0.514 | −0.509 | −0.821 |
| MGC2714 | −0.004 | 0.046 | −0.664 | −0.379 | 2.501 | 2.804 | 3.467 | 0.377 | −0.624 | −0.276 | 0.306 | −0.128 | −0.495 | −0.615 | −0.58 |
| MGC2716 [symbol] | 0.2315 | 0.7955 | −0.5285 | −0.3825 | 2.4405 | 2.7465 | 3.4535 | 0.0995 | −0.4785 | −0.0135 | 8.2615 | −0.1855 | −0.3515 | −0.6405 | −0.6875 |
| MGC2716 [symbol] | −0.1075 | 0.1955 | −0.7445 | −0.6545 | 1.9825 | 2.9235 | 3.6155 | 0.7885 | −0.3215 | −0.3265 | −0.3795 | 0.0615 | −0.4085 | −0.5095 | −0.9815 |
| MGC2716 [symbol] | 0.994 | 0.022 | −0.255 | −0.455 | 2.156 | 3.026 | 2.809 | 0.403 | −0.592 | −0.165 | 0.391 | −0.022 | −0.096 | −0.457 | −0.188 |
| MGC2716 [symbol] | −1.2775 | 0.5575 | 0.2055 | −0.2965 | −0.2645 | −0.7315 | −0.3725 | −0.2085 | −0.3045 | 0.1975 | 0.0375 | 0.6635 | 0.6785 | 0.4325 | 0.6315 |
| MGC2716 [symbol] | −0.003 | −0.768 | −0.598 | −0.471 | −1.052 | −0.061 | 0.044 | −0.177 | −0.578 | −0.014 | −1.435 | −0.364 | −1.087 | −1.2 | 0.204 |
| MGC2887 [symbol] | 0.529 | −0.443 | −0.217 | −0.205 | 0.166 | −1.053 | −0.801 | 8.493 | 0.29 | 0.748 | 0.338 | 1.611 | 0.818 | 0.868 | 0.998 |
| MGC3212 [symbol] | −8.834 | −0.844 | −0.742 | −0.972 | −1.179 | 0.22 | 2.502 | 1.266 | −0.379 | −0.915 | 1.858 | 2.474 | 0.283 | 1.552 | 0.812 |
| MGC3321 [symbol] | 1.3565 | 0.3045 | −0.2015 | 0.3795 | −0.2785 | 0.8525 | 1.4825 | 0.9045 | −0.2065 | 0.3385 | 2.5505 | 1.7175 | 0.5775 | 0.9405 | −0.0475 |
| MGC3484 [symbol] | −0.7435 | 0.8015 | −0.1735 | −0.7795 | 3.0285 | −0.5435 | −0.2545 | 0.2225 | 0.5945 | 0.4985 | 1.0365 | 0.8225 | 0.9745 | −0.5805 | −0.2445 |
| MGC3492 [symbol] | −1.638 | −0.692 | −0.769 | −1.994 | −1.623 | −1.031 | −0.287 | 0.341 | 0.137 | −1.619 | −0.157 | 0.658 | −0.747 | −0.162 | −0.543 |
| MGC4021 [symbol] | −0.734 | −0.15 | −0.271 | 1.248 | −0.944 | −0.852 | 0.046 | −0.35 | −0.516 | 0.032 | 0.318 | 8.57 | 0.022 | 1.46 | 1.109 |
| MGC4251 | 0.5045 | −0.0995 | −0.9335 | −0.8005 | −0.3195 | 0.1885 | −1.1015 | 0.1145 | 1.0775 | 0.3425 | 0.3515 | −0.4255 | 0.0745 | −0.5025 | −0.0235 |
| MGC4308 | −1.147 | −0.808 | 0.558 | −1.013 | −1.472 | −0.688 | −1.169 | −0.432 | 0.453 | −0.423 | 0.025 | 1.25 | −0.482 | −1.404 | −1.057 |
| MGC4800 | −1.7315 | −1.4705 | −1.0345 | −2.1725 | −1.7705 | −1.6855 | 0.2365 | 0.1295 | −0.0855 | −1.8215 | 2.2185 | 4.9365 | 3.1845 | 1.6445 | 0.6345 |
| MGC4659 | −0.916 | 0.681 | 0.724 | 0.897 | 0.078 | 1.036 | 1.35 | 0.581 | 0.929 | −0.111 | 0.218 | 1.942 | −0.258 | 0.509 | 0.27 |
| MIA | −0.4195 | 1.0665 | 0.9995 | 1.4545 | 8.5825 | 1.4915 | 1.6845 | 0.7465 | 1.0345 | −0.6005 | 8.5855 | 2.3355 | −0.2335 | 1.0355 | 0.5515 |
| MID1 | −1.4235 | 1.4195 | 1.4675 | 1.8785 | 0.7595 | 1.1415 | 1.0755 | 0.4625 | 1.4255 | 0.0035 | 0.5895 | 1.8835 | −0.0535 | 0.7595 | 0.9935 |
| MID1 | 1.199 | 0.99 | 0.494 | 1.47 | 1.105 | −0.269 | −0.315 | −0.398 | −0.145 | 0.398 | 0.949 | 0.272 | −1.086 | 0.58 | 0.953 |
| MID1 | −0.1815 | 0.8775 | 0.2335 | 0.5325 | 2.1025 | −0.1135 | 1.8865 | −0.2135 | −0.0355 | −0.1685 | 0.8465 | 0.6785 | −0.3745 | −0.3695 | 0.0145 |
| MLF1IP | 2.8335 | 3.8625 | 3.1125 | 2.2885 | 5.4875 | −0.4855 | 2.7555 | −0.1755 | 8.4295 | 2.6945 | 3.1145 | 3.0935 | 1.6985 | 2.1875 | 1.5905 |
| MLSTD1 | −2.238 | −1.126 | −0.974 | −0.708 | −2.247 | −2.939 | 2.44 | 1.1 | 0.027 | −2.492 | 1.305 | 2.778 | −0.845 | 1.23 | 1.888 |
| MMP1 | −0.049 | 0.426 | 0.185 | 0.247 | 0.258 | −0.324 | −0.531 | −0.837 | −0.042 | −0.448 | −0.512 | −0.473 | −0.832 | −0.358 | −0.321 |
| MMP7 | 0.7915 | −0.0815 | 0.0335 | −0.0855 | −0.3515 | −0.0455 | −0.0425 | 0.0055 | −0.1285 | 0.3755 | 0.4735 | 0.7615 | −0.2745 | 0.4815 | 0.7795 |
| MNAT1 | −0.6045 | 0.1425 | 0.1815 | 1.8915 | −0.1865 | 0.6335 | 0.3415 | −0.0085 | 0.5085 | 0.3045 | −0.1165 | 0.3405 | 1.1505 | 0.6345 | 1.3095 |
| MRPL14 | 0.079 | 0.657 | −0.378 | 0.573 | 0.18 | 1.24 | 0.696 | 0.339 | −0.364 | −0.365 | −0.257 | 0.338 | −0.502 | −0.168 | 0.607 |
| MRPL15 | −0.2565 | −0.0585 | −0.6125 | −0.2575 | −0.6615 | −0.1895 | −0.7895 | −0.2955 | −0.4505 | 0.1346 | −0.3255 | −0.1505 | 0.5385 | −0.5445 | 0.2725 |
| MRPL27 | −0.0815 | −8.0765 | −0.4645 | −0.7715 | −0.6275 | −0.2035 | −0.5885 | −0.3615 | −0.2715 | 0.2065 | −1.8245 | −0.2255 | −0.7455 | −0.0905 | −0.2805 |
| MRPL34 | 1.043 | −0.26 | −0.239 | 0.001 | 0.551 | 2.633 | 1.606 | −0.087 | −0.245 | 1.603 | −0.527 | 0.042 | −0.491 | −0.242 | −0.307 |
| MRPL43 | 0.0885 | 0.1785 | 0.3565 | 0.1895 | −0.0405 | −0.1885 | 0.4155 | 0.0515 | −0.0025 | −0.3785 | 0.0485 | 0.3245 | −0.1945 | 0.6405 | 0.6505 |
| MRPL45 | −0.186 | 1.114 | 0.14 | 0.132 | 0.146 | −0.332 | −0.278 | −0.328 | −0.183 | 0.203 | 0.304 | 0.185 | −0.075 | 0.685 | 0.653 |
| MRPL48 | −0.5115 | 0.5685 | −0.0905 | 0.0235 | −0.0525 | −0.4795 | −0.0665 | −0.2475 | −0.2625 | 0.0965 | −0.5245 | −0.0695 | −0.4485 | 0.2325 | 0.4765 |
| MRPL9 | 0.5375 | 0.1475 | −0.1876 | −0.2575 | −0.6635 | −0.0195 | 0.3095 | −0.0675 | −0.0855 | 0.1055 | 0.1325 | 0.6535 | −0.4685 | 1.2995 | 0.6015 |
| MRPS14 | −0.113 | 0.598 | −0.044 | 0.033 | −0.577 | −0.012 | 0.6 | −0.314 | −0.231 | 0.087 | 0.777 | −0.033 | 0.078 | 0.755 | 1.139 |
| MRPS15 | −0.048 | 0.05 | −0.05 | 0.421 | −0.396 | 0.064 | 0.405 | −0.177 | −0.22 | −0.171 | −0.289 | 0.185 | −0.313 | 0.56 | 1.018 |
| MRPS17 | 0.344 | 0.435 | −0.01 | 1.001 | −1.006 | 0.137 | 0.361 | 1.247 | −0.219 | −0.097 | −0.365 | 0.37 | 0.579 | 1.219 | 1.959 |
| MRPS24 | −1.3195 | 0.2515 | −0.3085 | 0.1995 | 0.1895 | 0.2025 | −0.8165 | −0.0195 | −0.4195 | −0.2115 | −0.4715 | 0.0015 | −0.9365 | −0.1075 | 0.1005 |
| MRPS25 | 1.444 | −0.67 | 0.563 | 0.058 | 0.34 | 0.744 | −0.853 | 0.232 | 0.047 | −0.051 | −0.552 | −0.396 | −0.803 | −0.441 | 0.888 |
| MRPS31 | −0.375 | −0.37 | −0.316 | −0.488 | −0.126 | 1.787 | 0.507 | 2.785 | 0.06 | −0.061 | −0.307 | −0.176 | 0.205 | −0.475 | −0.308 |
| MRPS6 | −0.1455 | −0.0335 | −0.3825 | 0.3955 | 0.9645 | 0.4385 | −0.0825 | −0.4665 | −0.5365 | −1.0005 | 0.3205 | 0.4455 | −0.3045 | 0.7675 | 0.1585 |
| MS4A1 | −0.2945 | −0.3235 | −0.0095 | −0.0905 | −0.0275 | −0.4735 | −0.7825 | −0.4525 | −0.3305 | −0.7935 | −0.1765 | −0.4195 | −0.6775 | 0.3255 | −0.5075 |
| MSH2 | 1.266 | 0.152 | −0.429 | 0.275 | −1.127 | −0.501 | 1.622 | −1.183 | −0.147 | −0.207 | −1.227 | −0.492 | 0.702 | −0.096 | 1.273 |
| MSH3 | −0.261 | −0.495 | −0.321 | 0.533 | 0.973 | −0.344 | 0.784 | 0.193 | 0.032 | −0.767 | −0.271 | −0.04 | −0.033 | 0.259 | −0.981 |
| MSMB | 0.359 | 0.313 | −0.347 | 0.378 | 0.87 | 0.192 | 0.092 | −0.344 | −0.214 | 0.008 | −0.05 | −0.555 | −0.014 | 0.283 | −0.148 |
| MTA1 | 0.7215 | 0.1425 | −0.1815 | 0.4315 | −0.2285 | 0.0375 | −0.2655 | −0.0375 | −0.2875 | −0.5825 | −0.9115 | −0.8465 | −0.7875 | 0.1955 | −0.7355 |
| MTA3 | 0.571 | −0.515 | 0.155 | −0.504 | 0.485 | 0.312 | −0.544 | −0.036 | 0.008 | 0.183 | 0.797 | 0.816 | 0.194 | 0.319 | 1.778 |
| MTAC201 | 0.044 | 0.513 | −0.234 | 0.668 | −0.058 | −0.265 | 0.229 | −0.377 | 0.176 | −0.416 | −0.051 | 0.098 | −0.203 | 0.052 | 0.271 |
| MTCH1 | −1.4275 | 0.4335 | 0.0535 | 0.6425 | 0.2055 | 0.8965 | 0.2835 | −0.4635 | 0.3055 | 0.2125 | 0.5135 | 0.7985 | 0.1205 | 0.5525 | 1.0845 |
| MTHFD1 | −1.2305 | −0.1505 | −0.5315 | −0.3355 | −1.8225 | −0.9415 | −0.0295 | −0.1525 | 0.5545 | 0.3685 | −0.0235 | −1.4595 | −0.3385 | −2.9845 | −3.5585 |
| MTMR2 | 0.6995 | −0.7225 | −0.1515 | 0.1575 | −0.4255 | 0.6575 | 2.0435 | 0.3075 | −0.6645 | −0.0805 | 0.3305 | −0.1965 | 0.5535 | 0.1965 | −0.7885 |
| MUC1 | 0.684 | −0.035 | −0.229 | −0.97 | −0.84 | −0.34 | 0.055 | 0.413 | 0.011 | 1.009 | 0.063 | 1.237 | 0.446 | −0.318 | −0.994 |
| MUC20 | −1.037 | −0.048 | −0.499 | −0.545 | 0.029 | −0.711 | −0.338 | −0.54 | −0.425 | 0.421 | −0.372 | −0.137 | −0.021 | −0.061 | −0.324 |
| MUM1 | −1.192 | −1.391 | −0.628 | −0.846 | −1.309 | −1.761 | 3.066 | −0.037 | 1.041 | −0.746 | −3.004 | −2.38 | −3.151 | −2.338 | −1.158 |
| MXI1 | −0.023 | −1.546 | −1.075 | 0.126 | −0.764 | −0.437 | −0.697 | 0.812 | 0.968 | −0.359 | 2.891 | 1.692 | 2.658 | −0.372 | −0.362 |
| MYB | 1.081 | 0.219 | 0.219 | 1.148 | 0.396 | 0.444 | 0.244 | 0.312 | 0.451 | 0.004 | 1.689 | 0.208 | 0.252 | 2.04 | 0.746 |
| MYBL1 | 0.785 | 0.485 | 0.776 | 1.018 | 0.103 | 0.49 | 0.625 | 0.104 | −0.294 | 0.424 | 0.789 | 0.648 | 1.333 | 0.741 | 1.294 |
| MYBL2 | −0.208 | −0.331 | −0.147 | −1.01 | 1.24 | −0.408 | −0.446 | −0.569 | 0.357 | 0.954 | −1.875 | −1.45 | −1.181 | −1.225 | −0.883 |
| MYO10 | −1.5355 | −0.4885 | −0.4915 | −1.3985 | 0.1295 | −0.3365 | 0.2085 | 1.2135 | −0.3485 | −0.2405 | −0.1635 | −0.0335 | 0.3105 | 0.0275 | 0.3715 |
| MYO6 | 0.4325 | −1.5085 | 0.4005 | −2.6185 | 1.2765 | −0.3285 | −2.1835 | 0.7895 | 1.3945 | 0.5815 | 1.1455 | −1.5925 | 2.3795 | −1.8635 | −1.5855 |
| MYST3 | 0.0455 | 0.0165 | 0.1095 | −0.1155 | −0.2035 | −0.2425 | 0.0185 | 0.7365 | 0.1035 | 0.1925 | 0.2845 | −0.1015 | 0.3435 | 0.1125 | −0.1285 |
| NALP2 | 0.6815 | 0.7205 | 0.4695 | 0.0015 | 0.9385 | 0.4985 | 0.1795 | 0.0955 | −0.0375 | −0.0535 | −0.5235 | −0.7385 | −0.4965 | −0.9625 | −0.1835 |
| NALP6 | 0.9325 | 0.0815 | 1.1715 | 0.4515 | −0.2505 | 0.9775 | 0.3085 | 0.3845 | 0.1255 | −0.0555 | 0.2745 | 0.4605 | −0.5305 | 0.4885 | 0.4915 |
| NANS | −0.162 | −0.552 | −0.256 | −0.592 | 0.099 | −0.244 | −0.227 | 0.872 | −0.203 | −0.281 | −0.36 | −0.723 | −0.494 | −0.547 | −0.678 |
| NARF | −0.84 | 0.899 | 0.637 | −0.113 | 0.201 | −0.371 | −1.044 | −0.185 | 0.54 | 0.004 | 1.779 | 0.481 | 0.08 | 0.139 | 2.197 |
| NAT2 | −0.4655 | 0.1215 | 0.1245 | −0.2715 | 0.1875 | −0.3355 | −0.0965 | 0.3925 | 0.2715 | 0.8235 | 1.0365 | 0.4865 | 0.0105 | −0.3775 | 0.4855 |
| NAV2 | 2.854 | 1.216 | −0.818 | 0.168 | 0.254 | 0.087 | −0.642 | 0.28 | −0.325 | −1.325 | −0.995 | −0.786 | −1.195 | −1.645 | −0.556 |
| NDE1 | 0.5795 | −2.0445 | −0.9705 | −0.5605 | −0.4495 | −0.2885 | −1.3495 | 0.4965 | 0.3475 | −0.7315 | 0.9055 | 1.2995 | 1.9795 | 0.0195 | 0.5325 |
| NDP52 | −0.205 | −0.122 | −1.025 | −1.212 | −0.191 | −0.777 | −0.6 | −0.427 | −0.513 | 0.292 | −1.005 | −0.595 | −0.145 | −0.468 | −0.305 |
| NDRG2 | 0.647 | 0.789 | −0.121 | 0.382 | −0.043 | −0.296 | −0.057 | −0.093 | 0.015 | −0.274 | 0.876 | −0.462 | −0.737 | −0.599 | −1.294 |
| N0UFA7 | 0.4105 | 0.1385 | −0.3315 | −0.1985 | −0.3805 | −0.2045 | −0.3785 | −0.0325 | 0.2095 | 0.3015 | −0.6385 | 0.3155 | −0.3885 | −0.5005 | 0.3635 |
| NDUFB1 | 0.777 | −0.182 | 0.495 | 0.71 | 0.278 | −0.178 | 0.245 | −0.096 | −0.571 | 0.02 | −0.469 | 0.236 | −0.283 | −0.028 | 0.625 |
| NDUFB10 | −0.599 | −0.871 | 0.268 | 0.058 | −0.569 | −0.162 | −0.363 | 0.088 | −0.854 | −0.247 | −0.463 | −0.324 | −0.892 | 0.32 | −0.704 |
| NDUFB2 | −0.5245 | 0.3095 | −0.1815 | 0.0235 | −0.0145 | −0.1935 | −1.2625 | −0.5385 | −0.6425 | −0.6315 | −0.1245 | −0.7035 | −0.7145 | 0.0305 | 0.5235 |
| NDUFB5 | 0.9805 | 0.3305 | 1.1145 | 0.5675 | 0.7815 | 0.0495 | −0.5055 | −0.8275 | 0.5715 | −0.6405 | −0.4195 | −0.7535 | −0.9995 | 0.2035 | 0.0625 |
| NDUFB6 | 0.1975 | 0.1185 | 0.3355 | 0.1585 | −0.0475 | 0.3525 | 1.8545 | −1.2105 | 0.3645 | 0.4205 | 0.5945 | 0.4595 | −0.1505 | 0.7015 | 1.2095 |
| NDUFC2 | −0.9585 | 0.8975 | 0.3355 | 0.1415 | 0.8875 | −0.9045 | −0.0775 | −0.4665 | −0.4605 | 0.5435 | 1.7725 | 0.6835 | 0.0745 | 1.5305 | 1.0215 |
| NEBL | 0.742 | −0.088 | 0.607 | 0.841 | 0.979 | 0.524 | 0.53 | 0.547 | 0.416 | 0.194 | −0.21 | 0.08 | 0.268 | −0.047 | −0.818 |
| NEK2 | −1.493 | 0.887 | −0.343 | −0.917 | 0.521 | 0.37 | −0.382 | 2.263 | 1.427 | −0.8 | −0.353 | −0.795 | −0.858 | 1.709 | −0.881 |
| NEK6 | −0.8555 | −0.2185 | 0.0015 | 0.1225 | −0.4085 | −0.9205 | 0.0735 | 0.7015 | 0.1315 | −1.4655 | 0.1935 | −0.2785 | −1.4825 | −0.0815 | 0.7275 |
| NELL2 | 0.5245 | −0.6935 | 0.1025 | −0.1745 | 0.0975 | 0.5795 | 1.0425 | 0.8115 | −0.0955 | 1.3345 | −0.2035 | 0.3035 | 2.1145 | 1.1245 | 0.7045 |
| NEURL | −0.775 | 0.149 | 0.330 | 0.799 | −1.197 | −0.158 | −0.158 | 0.425 | 0.537 | 0.583 | −2.873 | −0.053 | −0.508 | 0.027 | −2.53 |
| NFE2L3 | −1.1685 | 0.3975 | 0.3225 | −1.5845 | −0.5985 | 0.5495 | 0.5235 | 0.4865 | −0.4385 | 0.4865 | 0.8495 | 1.1085 | 0.8475 | 1.5335 | −0.5685 |
| NFIA | −0.278 | −0.889 | −0.139 | −0.631 | 0.165 | 0.555 | 0.357 | 0.573 | 0.104 | −0.53 | −1.444 | 0.065 | −0.005 | −0.888 | −0.075 |
| NFIB | −1.0885 | 0.1815 | −0.0595 | −0.0625 | 0.8355 | −0.0315 | −0.1215 | −0.3585 | −0.3475 | −0.4025 | −0.4165 | −0.2695 | −0.5515 | −1.0845 | −0.4075 |
| NINJ1 | −0.2485 | −0.0965 | 0.0635 | −0.1755 | 0.1525 | 0.0355 | −0.0015 | 0.0605 | 0.0695 | 0.1805 | −0.0545 | 0.4955 | −0.1025 | 0.1845 | 0.2225 |
| NIPSNAP [symbol] | −0.1135 | 2.4165 | 0.0805 | 1.4135 | 0.1785 | −0.4905 | 0.7585 | 0.2285 | −0.3095 | −0.3785 | 0.2455 | 0.2145 | −0.2035 | 0.3075 | 1.1825 |
| NKX8-2 | 0.3985 | 2.4485 | −0.1515 | 1.2035 | −0.2975 | −0.5075 | 0.8395 | 0.2435 | −0.4775 | −0.4575 | −0.2035 | 0.3435 | −0.5345 | −0.1325 | 1.4105 |
| NME1 | 0.391 | 0.014 | −0.536 | −1.912 | −0.812 | −0.265 | −0.851 | 0.574 | 1.251 | −0.082 | −0.964 | −0.666 | −0.063 | −0.931 | −1.215 |
| NME2 | −0.5435 | −2.0465 | −1.5725 | 0.7825 | −0.6435 | −0.7395 | 1.0395 | −0.6575 | −1.9035 | −0.8776 | −1.1975 | 0.3335 | −1.1435 | −3.0515 | −0.2295 |
| NME3 | 0.0875 | 0.4695 | 0.4915 | 0.9485 | 0.9565 | 0.2455 | −0.2115 | −0.0545 | −0.4795 | −0.2665 | −0.1805 | −0.3835 | 0.1005 | 0.0605 | 0.7805 |
| NMES1 | 0.003 | 0.268 | 0.183 | 0.127 | 0.71 | −0.194 | 0.626 | 0.373 | −0.166 | 0.155 | 0.375 | 0.242 | −0.372 | 0.814 | 0.772 |
| NMI | 0.0075 | 0.5935 | −0.1655 | −0.2875 | 0.3165 | −0.1555 | −0.7915 | −0.3165 | −0.0695 | 0.1365 | −0.1095 | 0.0275 | −0.1875 | −0.0215 | 0.7725 |
| NOC4 | 0.8095 | 0.6865 | −0.0955 | −0.2925 | 1.7035 | 2.4715 | 1.8045 | 1.4315 | −0.2895 | 0.0375 | −0.1495 | 0.0585 | −0.0815 | −0.3625 | −0.3645 |
| N0LC1 | −0.8805 | −0.0065 | 0.8625 | 0.8045 | 0.6815 | 0.2585 | 0.1035 | 0.2035 | 0.1175 | −0.1385 | 0.5565 | 0.3065 | −0.0355 | −0.0725 | 1.1255 |
| NotFound | −0.0575 | 0.2535 | 0.4235 | −0.4065 | −0.1905 | 0.1055 | 0.1015 | −0.3455 | −0.0865 | 0.1195 | −0.8785 | −0.2535 | −0.4745 | −0.1235 | 1.0015 |
| NotFound | 0.82 | 0.154 | −0.195 | −0.387 | 1.307 | 1.724 | 2.171 | 0.154 | 0.107 | −0.02 | −0.127 | 0.051 | 0.197 | −0.234 | 0.037 |
| NotFound | 2.019 | −0.448 | −1.318 | 0.715 | 0.259 | −2.491 | −1.814 | −0.551 | −0.851 | 0.048 | −2.106 | −1.047 | −2.425 | −0.901 | 0.457 |
| NotFound | −0.319 | −0.604 | −0.796 | −0.706 | 0.866 | 1.739 | 1.179 | 2.505 | −0.155 | −0.211 | −0.241 | 0.189 | −0.252 | −0.705 | −0.33 |
| NotFound | 0.1395 | −1.0305 | −0.1235 | 0.5865 | 0.6485 | 0.5915 | 1.2925 | 1.3565 | −0.8655 | −0.7315 | −0.1965 | −0.2005 | 0.8275 | 1.0285 | 0.6485 |
| NotFound | −1.072 | −0.836 | −0.469 | −0.933 | −0.807 | −0.718 | 0.478 | 1.334 | 0.211 | −0.809 | 1.058 | 3.687 | 0.903 | −0.53 | −0.412 |
| NotFound | −0.0105 | 0.1235 | 1.0655 | 1.2665 | 0.0775 | 1.2575 | 0.9315 | 0.2545 | −0.4315 | −1.4055 | 0.3995 | 0.6165 | −0.0475 | 0.2835 | −0.7085 |
| NotFound | −0.788 | 0.17 | 0.14 | 0.029 | 0.88 | 0.365 | 0.116 | 0.981 | −0.803 | 0.315 | −0.208 | 1.505 | −0.076 | 0.732 | 1.17 |
| NotFound | 1.2725 | −0.4335 | −0.0975 | −0.1175 | −0.1805 | 0.7295 | 0.7395 | −0.2525 | 0.3625 | 0.9825 | 1.2705 | 0.8485 | 1.4185 | 0.9095 | 1.2495 |
| NotFound | −0.2135 | 0.0885 | 0.2015 | −1.0625 | 1.5475 | 1.4885 | 3.4105 | 0.4875 | −0.4685 | −0.2715 | −0.6885 | −0.4465 | −0.2815 | −1.7015 | −1.2435 |
| NotFound | 0.4905 | −0.1415 | −0.3285 | −0.3325 | −0.3125 | 0.2245 | 0.7535 | 0.9515 | 0.1835 | 0.1515 | 0.5556 | 0.9355 | 0.8005 | 0.4005 | 0.4465 |
| NotFound | −0.1795 | −0.0785 | −0.0835 | −0.2455 | −0.1115 | −0.1485 | −0.0575 | 0.0385 | 0.1205 | 0.0205 | 0.1215 | 0.0695 | 0.3345 | −0.0015 | −0.4995 |
| NotFound | −1.977 | −1.878 | −1.895 | −3.072 | −2.675 | −1.452 | −0.935 | −0.218 | 0.258 | −1.344 | −0.824 | −0.155 | 1.295 | −0.731 | −0.889 |
| NotFound | 0.885 | 0.042 | −0.188 | −0.309 | 0.208 | 0.002 | −0.288 | 0.036 | −0.371 | −0.089 | −0.272 | −0.169 | −0.512 | −0.534 | 0.447 |
| NotFound | 0.006 | −0.438 | −0.51 | 0.07 | 0.036 | 1.94 | −0.091 | −0.13 | 0.306 | −0.289 | −1.04 | −0.348 | −1.504 | 0.415 | −1.246 |
| NotFound | −0.3595 | 0.1155 | −0.0635 | −1.1185 | −0.1825 | −0.0585 | −0.5485 | −0.0565 | 0.0365 | 0.1145 | −0.7085 | 0.2045 | −0.5035 | −1.4645 | −2.2385 |
| NotFound | 0.058 | 0.506 | 0.009 | −0.77 | 1.018 | −0.257 | 1.647 | 0.337 | −0.218 | 1.025 | 0.381 | 9.361 | −0.05 | −0.15 | −0.588 |
| NotFound | −0.151 | 0.497 | −0.028 | 0.288 | −0.018 | −0.47 | −0.358 | −0.008 | −0.492 | −0.498 | −0.118 | 0.355 | −0.236 | 0.121 | 0.068 |
| NotFound | 0.8935 | −0.4405 | 0.1735 | −0.3375 | −0.0205 | −0.1805 | 0.0515 | 0.0245 | −0.0005 | 0.0605 | −0.3875 | −0.6415 | −0.1415 | 0.3475 | 0.4525 |
| NotFound | 0.3135 | −1.8705 | −1.6275 | −1.2735 | −1.0185 | −1.7185 | −1.9215 | −0.4415 | −0.3075 | −1.5405 | −1.8005 | −0.3215 | −1.5445 | −2.1895 | 0.5015 |
| NotFound | −0.101 | −0.548 | −0.624 | −0.788 | −0.769 | −0.011 | −0.278 | 0.489 | −0.209 | −0.728 | −0.872 | 0.171 | −0.5 | −0.487 | −0.575 |
| NotFound | −0.0305 | −0.4925 | 0.2035 | 0.8395 | −0.0735 | −0.4385 | −0.0295 | 0.2265 | −0.7285 | −1.1045 | 0.5395 | 2.1255 | 0.0005 | 1.1105 | 0.0375 |
| NotFound | −1.515 | −1.296 | −1.238 | −1.161 | 0.039 | −0.835 | −0.702 | 1.098 | −0.676 | −1.012 | −1.542 | −1.644 | −1.428 | −1.479 | −1.588 |
| NotFound | −0.999 | 0.435 | 0.474 | 0.502 | 1.576 | 0.613 | 0.346 | −0.067 | −0.077 | −1.753 | 1.063 | −0.589 | 1.363 | 0.506 | −0.653 |
| NotFound | −0.8685 | −0.0355 | 0.8085 | 1.9915 | 0.3815 | −0.9185 | 0.0515 | 0.3655 | 1.2845 | −0.4845 | −0.8915 | 0.2935 | 1.3835 | 1.2545 | 1.1685 |
| NotFound | −0.83 | 0.573 | −0.29 | −0.424 | −0.4 | −0.276 | 0.355 | 0.424 | −0.174 | 0.536 | −0.156 | 0.676 | 0.753 | 0.842 | 0.424 |
| NotFound | −0.9915 | 0.1835 | 0.1865 | 0.5035 | 1.1855 | 0.6055 | 0.4405 | −0.2215 | −0.0485 | −1.7685 | 1.0195 | −0.8095 | 1.1275 | 0.6305 | −0.8245 |
| NotFound | 0.148 | 0.325 | −0.507 | 0.183 | −0.268 | −0.41 | 0.476 | −0.682 | −0.506 | 0.159 | 2.905 | −0.655 | 2.493 | −0.044 | 0.448 |
| NotFound | −1.445 | −0.426 | −1.144 | −0.749 | −0.505 | −0.484 | −1.124 | −0.646 | −0.675 | −1.204 | −1.123 | −0.837 | −1.63 | −0.882 | −0.335 |
| NotFound | 0.3725 | 0.5645 | 0.0045 | 0.0145 | 2.4185 | 2.5825 | 2.8135 | 1.3395 | −0.3295 | −0.2215 | 0.2785 | 0.2435 | −0.1395 | −0.4255 | −0.7675 |
| NotFound | −1.4895 | 0.2645 | 1.5355 | 0.3475 | 0.3855 | 0.5405 | 1.6805 | −1.3475 | −0.4845 | −1.2055 | 0.2315 | −1.6815 | 0.4405 | 0.1345 | −0.3725 |
| NotFound | −0.1855 | 0.2805 | 1.3405 | 0.8145 | 0.8615 | 1.2205 | 0.5675 | −0.4235 | −0.3315 | −1.3585 | 0.2255 | −0.9855 | 0.9225 | 0.4445 | −0.2335 |
| NotFound | −0.9305 | 0.3075 | 0.3185 | 0.7675 | 1.2345 | 0.3645 | 0.2905 | −0.4135 | −0.2685 | −1.2805 | 0.5545 | −0.5555 | 0.5225 | 0.7565 | −0.2905 |
| NotFound | −1.7665 | −0.7035 | −0.6895 | −0.7745 | −0.3455 | −0.2745 | −0.6515 | −0.5405 | −0.4355 | −0.0965 | −1.1755 | −0.2405 | −0.3025 | 0.3095 | −0.5845 |
| NotFound | −1.026 | 0.33 | 0.215 | 0.538 | 0.254 | −0.038 | 0.874 | −0.158 | −0.27 | −1.081 | 0.412 | −0.437 | 0.817 | 0.009 | 0.182 |
| NotFound | −1.9325 | −0.2135 | 0.3685 | 0.3505 | 0.8915 | 0.0435 | −0.1615 | −0.0015 | −0.4915 | −1.8545 | 0.3255 | −1.0425 | 0.8775 | −0.4585 | −0.4485 |
| NotFound | 0.4465 | 1.1685 | 1.0035 | 0.4945 | 0.7355 | −0.4105 | 0.0905 | −0.0045 | −0.2175 | 0.2085 | 0.6275 | 0.8495 | 0.3735 | 0.2235 | 0.4025 |
| NotFound | 0.2645 | 1.1285 | 1.2305 | 1.6905 | 0.6245 | −0.0095 | 1.3955 | −0.9435 | −0.8675 | −2.2055 | 0.4175 | −1.3285 | 1.3465 | 0.0325 | −0.8545 |
| NotFound | −0.0425 | −0.1685 | −0.0815 | −0.1155 | −0.3545 | 0.6405 | 1.3075 | −0.0195 | 0.0465 | 1.3525 | −1.3535 | −0.7135 | −0.5815 | −0.2995 | −0.9795 |
| NotFound | −0.286 | −0.638 | −0.888 | −1.004 | 0.155 | −0.49 | 0.81 | −0.812 | −0.018 | −0.537 | 1.212 | 4.54 | 2.402 | 3.058 | 3.715 |
| NotFound | 0.023 | −0.148 | −0.178 | −0.078 | −0.029 | −0.124 | 0.083 | −0.206 | −0.078 | 0.12 | 0.113 | 0.1 | 0.004 | −0.074 | −0.004 |
| NotFound | −0.007 | 0.056 | −0.04 | −0.399 | 2.2 | 3.204 | 2.95 | 0.539 | 0.251 | −0.377 | −0.011 | 0.395 | 0.072 | −0.445 | −0.308 |
| NotFound | −0.8615 | 0.1425 | 0.1015 | 0.4475 | 0.9205 | 0.4015 | 0.4785 | −0.3355 | −0.0865 | −1.2815 | 0.7785 | −0.4565 | 1.0515 | 0.6815 | −0.4305 |
| NotFound | −0.8135 | −0.0145 | −0.2755 | 8.1775 | −0.4185 | −0.8035 | 0.5195 | −0.3175 | −0.1275 | 0.5895 | 1.5015 | −0.2535 | −0.0925 | −0.1925 | 1.5825 |
| NotFound | 0.192 | 0.02 | 0.075 | −0.235 | 0.685 | 0.748 | 0.664 | 0.14 | 0.143 | −0.511 | 0.153 | 0.17 | 0.469 | −0.175 | −0.413 |
| NotFound | 0.3215 | 0.1005 | 0.3576 | −0.0765 | 0.2025 | −0.0405 | 0.3135 | 0.1005 | 0.1645 | −0.0965 | 0.6115 | −0.4785 | 0.4395 | 0.3425 | −0.2925 |
| NotFound | −0.5255 | −0.2895 | −0.1035 | −0.6975 | −0.3585 | −0.0165 | −0.3955 | 0.8505 | 0.0065 | −0.8955 | 0.1495 | 1.1585 | 0.7335 | 0.3355 | −0.3345 |
| NotFound | −0.2945 | −0.0095 | −0.2525 | −0.4835 | 0.4835 | −0.2475 | 0.1395 | −0.1935 | 0.4535 | −0.2265 | 0.8855 | 1.2785 | 0.8215 | 1.0095 | 0.9085 |
| NotFound | 1.3575 | 1.5255 | 0.4035 | 0.4205 | −0.0785 | −0.3485 | 0.0075 | 0.1305 | 0.2255 | 0.2485 | −0.5945 | 0.6655 | −0.2405 | 0.0135 | 0.2185 |
| NotFound | −0.558 | −1.081 | −0.64 | −0.169 | 0.743 | −0.186 | −0.19 | −0.806 | −0.132 | 0.041 | −0.047 | 0.133 | −0.773 | 0.087 | 0.434 |
| NotFound | 0.851 | −0.17 | −0.311 | −0.407 | 0.73 | −0.008 | −0.931 | 1.011 | 0.204 | 1.353 | −0.387 | −0.552 | −0.16 | −1.02 | 4.405 |
| NotFound | 1.6125 | −0.4475 | −1.1955 | 0.6535 | 0.2625 | −1.4045 | −1.3845 | −0.4945 | −0.8805 | −0.2625 | −1.9215 | −0.9325 | −1.7735 | −0.7805 | 0.4865 |
| NotFound | 0.3835 | 0.4215 | −0.6475 | 0.0285 | 0.3195 | −0.3395 | 0.0785 | −0.1185 | 0.0055 | 0.3025 | −1.3005 | −0.2685 | −0.7425 | −0.6275 | 2.5525 |
| NotFound | −0.9025 | −0.7795 | −0.5065 | −1.3155 | −0.9695 | −0.4165 | 0.9765 | −0.5345 | 0.2705 | −0.1305 | −2.1445 | −0.7135 | −0.8185 | −1.2765 | −1.6415 |
| NotFound | −1.0265 | −0.5645 | 0.2125 | 0.3545 | −0.2105 | 0.1335 | −0.1355 | 0.4505 | −0.5045 | −0.1705 | 0.5055 | −0.6755 | −0.6535 | −0.0875 | −0.7765 |
| NotFound | −3.244 | −1.958 | −2.041 | −1.34 | −3.321 | −2.604 | −0.599 | −0.275 | 0.387 | 1.24 | 0.776 | 0.833 | 0.677 | 0.362 | −1.162 |
| NotFound | −0.519 | −0.26 | −0.18 | 0.394 | −0.179 | −1.057 | 1.027 | 2.123 | −0.181 | −0.607 | −0.683 | 0.943 | 0.335 | −0.917 | 0.209 |
| NotFound | −0.0705 | −0.0205 | −0.0035 | −0.0405 | 0.3795 | 0.8415 | 0.5595 | −0.0385 | 0.0875 | 0.0045 | −0.2205 | −0.1585 | 0.2755 | 0.0065 | −0.5855 |
| NotFound | −1.093 | 1.254 | 1.372 | −0.454 | −1.036 | 0.228 | −0.558 | 1.122 | 0.522 | 1.999 | −1.752 | 0.832 | 1.036 | −1.286 | −1.055 |
| NotFound | −0.815 | −0.23 | 3.003 | 0.475 | −0.022 | 0.21 | 0.748 | −0.283 | −0.815 | −0.832 | −0.574 | −0.356 | −0.817 | −0.304 | 0.27 |
| NotFound | −0.0355 | −0.7055 | −0.7035 | −0.6295 | −0.6295 | −0.5525 | −0.3105 | 2.7915 | 2.1265 | −1.1275 | 0.0085 | 0.4505 | 0.2776 | 0.6275 | −0.2845 |
| NotFound | −0.091 | −0.046 | 0.068 | −0.113 | −0.387 | 0.275 | −0.106 | 0.167 | −0.242 | 0.123 | −0.059 | −0.58 | 0.101 | −0.066 | −0.055 |
| NotFound | −0.311 | 0.01 | 1.431 | 0.72 | 0.934 | 1.429 | 0.785 | −0.444 | −0.34 | −2.161 | −0.105 | −1.107 | 0.907 | 0.572 | −0.457 |
| NotFound | 0.5185 | −0.2045 | 1.0225 | 0.1785 | 0.8835 | 0.4185 | 0.2335 | −0.2155 | 1.2125 | −0.7925 | 0.0525 | −0.8825 | −0.8025 | −0.5505 | −0.2855 |
| NotFound | −0.499 | −0.752 | 0.201 | −0.857 | −0.059 | 0.3 | 0.066 | −0.103 | 0.113 | 0.015 | −0.148 | −0.73 | −0.632 | −0.585 | −0.286 |
| NotFound | 0.4825 | −0.2045 | 0.0025 | −0.4555 | 1.2075 | −0.0485 | −0.0015 | 0.4285 | 0.2455 | 0.8505 | 0.5375 | −0.0255 | 0.4285 | 0.8545 | 0.8845 |
| NotFound | 0.4975 | 0.3415 | 0.3075 | 0.2635 | 0.6375 | 0.1585 | 0.8105 | 0.1315 | 0.2525 | 0.2895 | 0.4665 | 1.3655 | 0.2385 | −0.3415 | −0.8885 |
| NotFound | −1.09 | 0.317 | 0.264 | 0.367 | 1.153 | 0.475 | 0.329 | −0.209 | −0.105 | −1.478 | 1.005 | −0.713 | 1.391 | 0.352 | −0.682 |
| NotFound | 0.2255 | 0.5495 | −0.0645 | 0.3825 | 0.5535 | −0.0685 | −0.2665 | 0.1245 | −0.3235 | 0.3135 | 0.4025 | 0.2815 | 0.4715 | 0.4755 | 0.4235 |
| NotFound | −0.09 | 0.258 | 0.014 | −0.366 | 0.452 | −0.569 | −0.316 | 0.33 | 0.383 | −1.237 | −0.899 | −0.54 | −0.281 | −1.956 | −2.034 |
| NotFound | 3.384 | −0.444 | −0.263 | 0.75 | 0.275 | −0.292 | −0.254 | 0.088 | 0.044 | −0.335 | −0.188 | −0.176 | −0.813 | −0.287 | −0.678 |
| NPDC1 | 0.4555 | 0.2885 | 0.1895 | 0.1395 | 0.1885 | 0.0355 | −0.1795 | −0.2615 | −0.7885 | 0.1225 | 0.3725 | −0.4595 | 0.3715 | 0.1805 | 0.1575 |
| NPEPPS | −0.041 | −0.704 | 0.262 | −0.204 | −0.14 | 0.896 | −0.887 | 0.172 | −0.916 | 0.327 | −1.09 | 0.42 | −0.922 | −0.924 | −0.487 |
| NPY | 1.182 | −0.419 | 0.381 | 0.737 | 0.551 | 0.641 | 0.408 | −0.443 | 0.034 | 0.327 | −0.427 | 0.273 | 1.834 | 0.914 | 0.684 |
| NQO3A2 | −1.387 | −0.887 | −0.265 | −0.402 | −0.774 | 0.233 | −1.118 | 0.343 | −0.53 | −2.325 | −2.88 | −1.513 | −1.795 | −1.427 | −0.743 |
| NRAS | −3.289 | −1.014 | 0.273 | −1.048 | 8.895 | −0.619 | 0.708 | −1.427 | 0.493 | 1.043 | −2.846 | −1.99 | −0.368 | −0.873 | −2.999 |
| NRIP1 | 2.1905 | −0.5295 | 0.1425 | −0.6655 | −0.5945 | −0.0075 | −0.9975 | −1.0955 | −0.3935 | −0.2245 | −0.4715 | −1.4645 | −0.9895 | −0.4045 | 0.1276 |
| NTN4 | −0.0005 | 0.4465 | 0.2885 | 1.1505 | −0.2875 | −0.4175 | 0.2825 | −0.7255 | 0.6715 | 1.5475 | 0.4035 | 0.9645 | 0.4395 | 1.4905 | 1.7715 |
| NUCB2 | −0.8735 | −0.9845 | −0.9935 | −0.5385 | −1.1255 | −0.4445 | −1.5945 | −0.4915 | −0.0455 | −0.4295 | −1.3625 | −0.6095 | −0.9535 | 0.1785 | −0.1135 |
| NUDT5 | −0.631 | 0.027 | −0.526 | 1.348 | −0.021 | −0.545 | 0.269 | −0.225 | −1.204 | 0.351 | 0.586 | 0.012 | −0.221 | 0.249 | 0.535 |
| NUDT6 | 0.409 | 0.303 | 0.064 | 0.054 | 0.17 | −0.364 | 0.337 | 0.312 | 0.198 | −0.218 | 1.044 | 0.489 | −0.311 | 0.269 | 0.828 |
| OCIAD2 | 0.6255 | −1.3065 | −0.3785 | −1.2515 | −0.8365 | −1.0945 | −0.3425 | 0.4065 | −0.0415 | 1.7025 | 0.5385 | 0.6825 | 4.5575 | 0.9075 | 0.4005 |
| OFD1 | 0.2415 | −1.8865 | 0.3575 | −0.5205 | −1.4845 | −0.8855 | −0.5335 | 0.4915 | 1.0225 | −0.3195 | 0.3655 | 1.9925 | 2.2295 | 0.0095 | 0.4855 |
| OGFRL1 | 2.355 | 0.252 | 1.764 | 0.081 | −0.182 | 2.546 | 1.173 | 0.112 | −0.124 | 0.558 | −0.242 | 0.103 | 0 | −0.402 | −0.84 |
| OPLAH | 0.4785 | −0.1475 | 0.4975 | 0.4445 | −0.0555 | 1.0815 | 1.8395 | 1.4235 | 0.1235 | 3.1285 | 1.1025 | 0.9665 | 1.4505 | 0.7925 | 0.8615 |
| ORMDL3 | 1.301 | −0.569 | −0.07 | −0.221 | −0.757 | 0.619 | −0.035 | 0.16 | −0.077 | −0.379 | −0.621 | −1.424 | −1.269 | 0.184 | −1.532 |
| OSBPL3 | 0.869 | 0.157 | 0.349 | 1.088 | 0.288 | 0.575 | 0.42 | −0.2 | −0.251 | 0.162 | 0.006 | −0.29 | 0.149 | −0.991 | 0.582 |
| OSR2 | −0.2785 | −0.0825 | −0.2145 | −0.6175 | 0.5485 | 1.1415 | 2.0045 | 0.2595 | −0.0625 | −0.3395 | −0.1155 | −0.1435 | −0.0975 | −0.4485 | −0.4165 |
| OSTF1 | 1.657 | 0.054 | 1.011 | 0.282 | −0.345 | 0.949 | 0.973 | 2.151 | −0.051 | 0.615 | −1.202 | −0.657 | −1.89 | 0.232 | 1.325 |
| P2RX1 | 0.4455 | 0.4025 | −0.3125 | −0.9045 | 1.5315 | 3.1245 | 1.6355 | 0.1475 | −0.1625 | 0.2745 | 0.1785 | −0.6185 | −0.1575 | −1.1185 | −1.1815 |
| P2RY2 | 0.788 | 1.732 | 0.208 | −0.182 | −0.57 | 0.804 | 1.249 | 0.22 | 0.355 | 0.015 | 0.358 | 1.25 | 1.859 | 0.194 | −0.301 |
| PACAP | −0.718 | −0.023 | 0.367 | 0.153 | 0.763 | −0.184 | 0.622 | 0.518 | 0.422 | −1.12 | 0.172 | −0.014 | −0.197 | −0.715 | 0.023 |
| PADI2 | 1.7285 | 0.1715 | 2.1535 | 0.4475 | −0.5895 | 1.8895 | 0.4305 | 0.0615 | 0.3205 | 2.1785 | 1.8575 | 0.1455 | −0.7175 | 0.6345 | −1.0225 |
| PAPD5 | −0.357 | 0.065 | −0.031 | −0.578 | −0.073 | 0.004 | 0.577 | −0.004 | 0.423 | 0.285 | 0.067 | −0.189 | 0.37 | −0.104 | 0.038 |
| PAPSS2 | −0.302 | 0.314 | 0.258 | −0.566 | 0.233 | −0.017 | −0.315 | 0.137 | 0.117 | 0.575 | 0.033 | 0.173 | 0.71 | 0.567 | 0.233 |
| PARD8A | −0.63 | −0.851 | 0.53 | 0.312 | 0.58 | 0.223 | 0.393 | 0.734 | −0.643 | −0.873 | 0.175 | −0.29 | −0.606 | −0.215 | −0.658 |
| PARP1 | −0.119 | −0.456 | −0.696 | −0.174 | −0.018 | −0.254 | 0.169 | 0.819 | −0.25 | −0.282 | 0.172 | 0.147 | −0.377 | 0.178 | −0.164 |
| PARP9 | −0.1535 | −0.0065 | 0.4005 | 0.5625 | 1.2075 | 0.6995 | 0.1385 | 0.3075 | −0.2265 | −0.7565 | −0.4305 | −0.4165 | −0.8225 | −1.2335 | −0.6235 |
| PAXIP1L | 0.4635 | −0.0885 | 0.0875 | −0.0345 | 0.0275 | −0.1275 | −0.1275 | −0.0655 | −0.5885 | 0.1435 | 0.1925 | 0.2085 | 0.9225 | 0.4495 | 1.1205 |
| PBX3 | −0.0545 | 0.1795 | 0.4405 | 0.2565 | 0.1095 | −0.0455 | −0.2545 | 0.6675 | −0.3345 | −0.6445 | −0.5785 | −0.1735 | 0.1715 | 0.1795 | 1.8175 |
| PCDHB11 | 1.812 | −1.098 | −0.979 | 0.851 | −1.555 | 1.045 | 0.963 | −0.407 | −0.67 | 0.467 | 1.191 | 1.104 | 2.001 | −1.88 | 2.548 |
| PCDHB16 | −0.818 | −0.312 | −0.482 | −0.795 | 0.761 | −0.426 | −0.638 | 0.943 | −0.928 | −0.943 | −0.672 | −0.377 | −1.227 | 0.107 | −0.831 |
| PCDHB2 | 0.2315 | 0.1215 | −0.1145 | 0.2405 | 0.3355 | 0.5275 | −0.4245 | −0.1345 | 0.1275 | −0.1475 | 0.3025 | −0.0825 | −0.5305 | −0.0445 | 0.8095 |
| PCM1 | 0.4555 | −0.1335 | −0.0705 | −0.1075 | 0.7295 | 1.2855 | 0.9905 | 1.7315 | 0.1325 | 0.1175 | 0.7985 | 1.1605 | 0.6005 | −0.1255 | −0.1425 |
| PCNA | −1.117 | −0.954 | 0.473 | 0.818 | 0.304 | −0.252 | −0.017 | −0.144 | −0.845 | −1.099 | 0.663 | −0.584 | −1.542 | 0.247 | −0.577 |
| PDCD1 | 0.0095 | −1.2285 | −1.7615 | −1.6745 | −1.3545 | −1.0325 | −1.5115 | 0.8585 | 0.0395 | 0.2315 | 0.4385 | −0.1725 | 0.0535 | −0.5495 | −0.6955 |
| PDCD10 | −0.183 | 0.428 | −0.338 | 1.176 | 0.373 | −0.3 | −0.331 | 0.21 | 0.087 | 0.431 | 0.727 | −0.748 | −0.206 | 1.384 | 0.443 |
| PDCD4 | 0.5515 | 0.0135 | −0.3095 | −0.8135 | 0.0525 | 0.4445 | 0.2705 | −0.2725 | 0.0245 | −0.5525 | −1.4725 | −0.2685 | −0.1675 | −0.0325 | 0.4135 |
| PDCD5 | 1.6585 | 0.3475 | 0.3175 | 0.5665 | 0.8675 | 1.3575 | 0.0155 | −1.1665 | −0.3215 | 0.7365 | −0.3415 | 0.0475 | 0.7575 | −1.0825 | −0.7945 |
| PDCD8 | 0.7845 | 0.3475 | −0.0115 | 0.5585 | −0.2575 | 0.3665 | 0.3185 | 0.0345 | 0.4245 | −0.1815 | 0.1415 | 0.4815 | −0.0765 | 0.8285 | −0.0075 |
| PDE4DIP | 0.1035 | −0.2935 | −0.3525 | −2.1485 | 0.1485 | 0.1765 | −1.5485 | 0.0605 | 0.3775 | −0.0895 | −1.6445 | −0.3805 | −1.8745 | −0.0275 | −1.1485 |
| PDHA1 | −1.29 | −1.108 | −0.452 | 0.86 | 0.767 | −1.27 | 1.023 | −2.392 | −1.092 | −0.468 | 1.959 | 0.952 | 1.538 | 0.953 | 0.022 |
| PECI | −0.5565 | −0.2785 | −1.0475 | −0.4705 | −0.7395 | −0.4665 | −1.3875 | −0.3625 | −0.5825 | −1.1055 | −2.0215 | −1.1605 | −1.8565 | −1.7365 | 0.6205 |
| PEG10 | 1.4935 | −0.0605 | 0.5495 | 2.8335 | 0.4005 | −0.5125 | −0.4095 | −0.8125 | −0.3095 | 0.4125 | 0.7615 | 0.4895 | −0.7765 | 0.6285 | 0.8015 |
| PER2 | 0.252 | 0.158 | −0.593 | 0.282 | −0.005 | −0.248 | 1.513 | 0.165 | 0.358 | 0.064 | 0.138 | 0.332 | −0.216 | 0.071 | 0.187 |
| PERP | −0.7026 | −0.2705 | −0.1205 | −0.2535 | −0.2705 | −0.4165 | −0.5165 | 0.1795 | 0.2805 | −0.8855 | 0.3525 | −0.4825 | 0.0715 | −0.4465 | −0.3175 |
| PEX1 | −0.491 | 0.588 | −0.211 | 0.528 | −0.383 | −0.26 | 0.142 | −0.091 | −0.111 | 0.394 | 0.083 | 0.323 | 0.577 | 0.751 | 0.878 |
| PFAAP5 | 0.2325 | −0.6525 | 0.6455 | 0.1125 | 0.1445 | −0.6895 | 0.8625 | 1.2045 | 0.8195 | −0.9655 | 0.9525 | 0.1535 | −0.9805 | 0.6435 | 1.3965 |
| PFDN2 | 0.251 | −0.23 | 0.245 | −0.022 | 0.883 | −0.224 | −0.725 | −0.23 | −0.521 | −0.035 | −0.674 | −0.117 | −0.153 | −0.352 | 0.039 |
| PGM1 | −0.0225 | 0.3015 | 0.1385 | −0.4665 | 0.2985 | 0.2885 | −0.2195 | −0.5185 | −0.8775 | −0.5755 | −0.2365 | −0.2245 | −0.9605 | 0.0905 | −0.1205 |
| PGR1 | 1.5395 | −0.8885 | −0.1715 | 0.7755 | −0.3915 | 0.0275 | −0.7515 | −0.9015 | 0.1085 | −1.0305 | 1.0185 | 1.0835 | 2.1435 | 2.1085 | 2.5735 |
| PHAX | 0.661 | −0.765 | −0.313 | 1.484 | −0.78 | −0.148 | 0.823 | −0.893 | 0.536 | 1.689 | −0.527 | 0.404 | 0.988 | 0.89 | 2.212 |
| PHGDH | −1.5485 | −0.3355 | −0.3285 | −2.0545 | −0.5795 | −0.4095 | −0.3845 | 1.4455 | 0.5545 | 0.0095 | −1.4985 | −1.2425 | −1.8165 | −0.9895 | −2.2325 |
| PHYH | 0.5305 | −1.1405 | −0.4185 | −0.2585 | −0.5185 | −0.6135 | −0.3245 | 0.1285 | −0.1265 | −0.5505 | −0.7275 | −0.1745 | −1.0015 | −0.9435 | 0.1585 |
| PI85PA | 0.3845 | −0.4065 | −0.7305 | −0.7955 | 1.0925 | 2.7205 | 2.3865 | 0.6155 | 0.0015 | −0.0785 | 0.1755 | 0.1405 | 0.5505 | −0.0905 | −0.5495 |
| PIGT | 0.313 | −0.291 | −0.428 | −0.897 | −0.607 | −0.072 | −0.088 | 0.077 | −0.082 | −0.711 | −0.323 | 0.077 | 0.059 | −0.319 | −0.564 |
| PIM2 | 2.0115 | 0.4205 | 2.9235 | 0.5665 | 0.0865 | 0.4595 | 0.8105 | 0.4235 | 0.3135 | 0.4115 | −0.1725 | −0.3735 | −0.3685 | −0.2075 | 2.0465 |
| PISD | −0.043 | −1.881 | 0.604 | 3.012 | −1.441 | −0.086 | −1.144 | −1.751 | 0.627 | −0.692 | −1.746 | −1.991 | −1.895 | −1.082 | −2.164 |
| PITPNC1 | 0.5245 | 0.5735 | −0.2285 | −0.1025 | −0.4095 | 0.1165 | −0.1215 | −0.2915 | 0.2845 | 0.1815 | 0.7485 | 0.3805 | −0.3445 | 0.3375 | 1.2055 |
| PKIB | 0.157 | −0.29 | −0.581 | −1.012 | −0.434 | 0.829 | 0.585 | 0.329 | −0.223 | −0.155 | −0.784 | −0.334 | −0.591 | −0.664 | −0.07 |
| PKMYT1 | 0.001 | −0.001 | −0.142 | −0.732 | 0.451 | 0.889 | −0.158 | 1.997 | 0.818 | −0.245 | 9.277 | −0.822 | 0.425 | −0.949 | 0.013 |
| PLA2G4B | −0.2205 | −0.2255 | 0.1325 | −0.7835 | −1.2235 | 0.5925 | 0.1935 | 0.2145 | −0.1035 | −0.2215 | 0.9815 | 0.6815 | 0.1955 | −0.6655 | −0.3755 |
| PLAC8 | 1.568 | 0.718 | 0.535 | −0.41 | 0.423 | 0.231 | 0.453 | −0.028 | 0.26 | −0.214 | 1.346 | −0.528 | 0.58 | 0.58 | 0.53 |
| PLCB1 | 0.9795 | 0.1795 | 0.3735 | 1.3815 | −0.1995 | 0.1855 | 0.8775 | −0.7075 | −1.0565 | 0.0865 | −0.2055 | 1.3575 | −0.1535 | −0.2105 | 0.5085 |
| PLCXD1 | 2.16 | −0.001 | 0.963 | 0.308 | 0.065 | −0.114 | 0.672 | 0.357 | 0.334 | −0.575 | 1.431 | 2.098 | 1.448 | 1.085 | 0.518 |
| PLEK2 | −0.2755 | 0.5405 | 0.7775 | 0.4245 | 0.0245 | 0.1565 | 0.4385 | −0.0015 | −0.0495 | −0.0375 | 0.3185 | 0.3415 | 0.5625 | 0.2755 | 0.3505 |
| PLO0 | −0.0355 | −0.1235 | −0.0705 | −0.0785 | −0.0285 | 0.2975 | −0.6195 | −0.3215 | 0.2295 | 0.1175 | −0.2725 | −0.3575 | −0.0595 | −0.0035 | −0.5325 |
| PNAS-4 | −0.0745 | −0.0045 | 0.4645 | 2.3275 | 0.3365 | 0.8925 | −0.3415 | 2.3585 | 0.1285 | −0.0265 | −0.0145 | 0.6475 | 2.8705 | −0.0985 | 0.6765 |
| PNLIPRP2 | −1.1995 | −0.8545 | −0.3965 | −0.8775 | 0.7495 | −0.3925 | −0.5825 | 0.0985 | −0.7275 | 0.0645 | −0.2215 | 0.0485 | 0.6245 | −0.0675 | 0.1815 |
| PNMA3 | −0.5025 | 0.3555 | 0.0615 | 0.1545 | −0.8055 | −0.1595 | 0.3705 | 0.0765 | −0.4945 | −0.4155 | 0.2865 | 0.7185 | 0.6575 | 1.7145 | 0.5535 |
| POLB | 0.14 | −0.31 | −0.002 | 0.029 | −0.613 | −0.185 | 0.134 | 0.845 | −0.063 | −0.058 | 0.105 | −0.04 | 0.68 | 0.785 | −0.319 |
| POLR2F | 0.1365 | −0.2995 | −0.2725 | −0.8045 | 0.0005 | 0.1185 | 1.1575 | 0.1025 | −0.5435 | −0.4355 | 0.3855 | 0.0965 | 0.8935 | −0.2835 | −0.2505 |
| POLR2H | 0.2335 | −0.1555 | −0.3115 | 0.5985 | 0.6085 | 0.1095 | 0.1515 | 0.3335 | −0.3475 | −0.0025 | −0.0275 | −0.2845 | −0.8485 | 1.2895 | −0.2325 |
| POMC | 0.238 | −0.401 | −0.829 | 0.064 | −0.01 | −0.086 | 0.547 | −0.251 | 0.236 | 0.377 | −0.474 | −0.001 | −0.013 | 0.345 | 0.787 |
| POP4 | −0.061 | −0.141 | 0.088 | −0.824 | −0.537 | −0.189 | 0.256 | 0.487 | 0.306 | 0.161 | 0.155 | 0.928 | 0.067 | −0.352 | −0.024 |
| POP7 | 0.283 | −0.157 | 0.265 | −0.098 | 0.093 | 0.078 | 0.801 | 0.298 | 0.237 | 1.228 | −0.284 | 0.550 | −0.303 | 0 | −0.103 |
| PP1057 | 0.5175 | 0.8305 | 0.1925 | −0.1605 | 0.8255 | 0.0465 | 0.0145 | −0.0455 | −0.4195 | 0.5205 | 0.4705 | 0.2035 | −0.5605 | −0.3705 | 0.0175 |
| PPFIA1 | 0.036 | −0.449 | 0.283 | 0 | 0.012 | 0.4 | 0.075 | 0.065 | 0.176 | 0.273 | −0.148 | −0.015 | 0.507 | −0.325 | −0.445 |
| PPFIBP2 | −0.2585 | −0.1365 | −0.1735 | 0.2215 | −0.1105 | −0.1585 | 0.2175 | 0.1805 | −0.1035 | −0.8845 | −0.0045 | −0.0925 | 0.6175 | 1.8715 | −0.1916 |
| PPGB | 1.4575 | −0.1845 | −0.7045 | 0.2965 | −0.8915 | 0.2025 | −0.1455 | −0.4885 | −0.3985 | −0.8075 | 0.5245 | 0.0865 | −0.5945 | 0.2015 | −0.0645 |
| PPHLN1 | −0.1585 | 0.1445 | 0.0495 | −0.2005 | 0.0685 | −0.0045 | −0.5205 | 0.2745 | −0.4535 | −0.3175 | 0.3475 | 0.7775 | −0.1155 | 0.0915 | 0.9815 |
| PPIF | 0.3385 | 0.5105 | −0.8825 | −0.2615 | −0.2895 | −0.1485 | −0.1445 | 0.3485 | 0.3905 | −0.2495 | 0.2855 | 0.1905 | 0.4605 | 0.0145 | 0.5855 |
| PPIL1 | 0.2905 | −1.0975 | 0.0825 | 0.5455 | −1.0135 | −0.0045 | −0.4335 | 1.0945 | 0.6615 | 0.4775 | 0.3555 | 0.7455 | 1.8755 | 0.3545 | 1.1075 |
| PPOX | −1.839 | −1.175 | −0.648 | −0.742 | −0.637 | −1.962 | −1.096 | −1.8 | 0.01 | −0.561 | −1.72 | −1.352 | −0.404 | 0.067 | −0.716 |
| PPP1R16 [symbol] | 1.103 | 0.198 | −0.112 | −0.22 | 0.428 | 0.164 | −0.209 | −0.078 | −0.039 | 0.109 | 0.066 | −0.222 | −0.234 | −0.681 | 0.4 |
| PPP1R3C | −0.607 | 0.048 | −0.188 | −0.761 | 0.080 | −0.05 | −0.633 | −0.19 | −0.427 | −0.209 | −0.714 | −0.45 | 0.726 | −1.197 | −1.295 |
| PPP2R2D | −0.3885 | 1.4795 | 3.4585 | 1.9665 | 1.5725 | 2.4535 | 0.3525 | −0.9745 | 0.0205 | 1.7675 | 2.1785 | 4.2195 | 2.3965 | 2.3155 | 0.1255 |
| PPP2R5A | −0.117 | 0.099 | −0.337 | −0.456 | 0.011 | −0.196 | 0.434 | 0.071 | −0.37 | −0.026 | 0.56 | 0.57 | −0.016 | 0.49 | 0.962 |
| PRAME | 0.4705 | 0.3575 | −0.0475 | −0.0185 | 0.1335 | −0.0675 | 0.9835 | 1.4585 | −0.0625 | 0.4075 | 1.3705 | 0.5265 | −0.1395 | 0.7895 | 0.0735 |
| PRC1 | −0.188 | −0.252 | −0.209 | 0.188 | 0.068 | 0.589 | −0.204 | 0.53 | −0.035 | −0.132 | −0.149 | −0.59 | −0.487 | 0.06 | −0.36 |
| PRIM2A | 0.4365 | −0.0695 | 0.0176 | 0.0635 | 1.3085 | 1.8415 | 0.1705 | 2.4815 | 0.5085 | −0.2795 | 0.6545 | 0.0885 | 0.5265 | −1.3085 | −0.5055 |
| PRKACB | −1.355 | −0.932 | −0.784 | 0.025 | 1.166 | −0.784 | 0.369 | 0.693 | −0.438 | −0.254 | −0.149 | 0.22 | 0.207 | 0.699 | −0.313 |
| PRKCB1 | 0.285 | 0.003 | 0.294 | −0.071 | −0.467 | 0.189 | −0.211 | 0.087 | 0.088 | −0.278 | 0.032 | 0.052 | −0.027 | 0.45 | 0.251 |
| PRKCI | −0.483 | 0.121 | 0.002 | −0.473 | −0.002 | −0.404 | 0.267 | 0.539 | 0.742 | 1.297 | 1.405 | 2.6 | 0.642 | 0.943 | 1.37 |
| PRKRIR | −0.3165 | 0.6715 | −0.2185 | −0.2805 | −0.3645 | 0.4945 | 0.2535 | 0.8785 | 0.1715 | −0.0025 | 2.0225 | 0.9645 | 1.2035 | 1.3495 | 1.9285 |
| PRKWNK1 | −1.0325 | −0.6335 | 0.0795 | 0.4045 | −0.5785 | 0.6555 | −0.4455 | 0.0555 | −1.1435 | −1.5385 | −0.3305 | 0.3845 | 0.0375 | −0.7875 | −0.0065 |
| PRKX | −0.051 | −0.075 | −0.149 | −0.258 | 0.988 | 3.008 | 1.832 | 0.128 | 0.318 | −0.063 | −0.288 | −0.025 | 0.22 | 0.247 | −0.249 |
| PRNP | −0.0215 | −0.2975 | 0.1785 | 0.8865 | 0.0765 | 0.0025 | 0.2465 | −0.4855 | −0.4535 | 0.8205 | −0.3115 | 0.5455 | −0.4445 | 1.0105 | 2.4846 |
| PROL5 | −0.6805 | −0.0035 | 0.7275 | −0.2315 | −0.3125 | 0.0365 | 0.8305 | 0.0245 | −0.0505 | −0.1895 | −0.0905 | −0.1885 | −0.3365 | −0.8175 | −0.3225 |
| PRPF18 | 0.3335 | 0.1185 | −0.4995 | 0.3275 | −0.8095 | 0.1715 | −1.3825 | −0.5905 | 0.0905 | 0.0925 | 1.3505 | −0.6155 | 0.0165 | 0.2085 | 0.4915 |
| PRPSAP1 | −1.1275 | 0.2115 | −0.7005 | −1.2795 | −0.3905 | 0.1575 | 0.0555 | −0.8465 | −0.1415 | 0.1275 | 0.1775 | −0.5495 | −1.5285 | 0.5845 | −1.9625 |
| PRSS12 | −0.7105 | −1.1675 | −0.6315 | −0.7185 | −0.6375 | −1.0365 | 0.0825 | −0.4775 | −0.7955 | −0.8545 | −2.2555 | −0.8185 | 0.5305 | −0.7945 | −0.0675 |
| PRSS16 | 0.8395 | 0.4295 | −0.0475 | 1.1705 | 0.7805 | 0.1075 | −0.0835 | −0.3455 | −0.0365 | −0.8445 | −0.2735 | −0.7455 | −0.4035 | 0.1915 | 0.6735 |
| PSD3 | 0.6905 | 0.2505 | 0.2565 | 0.3485 | 0.4215 | 0.1485 | 0.3195 | 1.1065 | −0.2285 | 0.3355 | 0.2325 | 0.2795 | −0.0575 | 0.7535 | 0.6775 |
| PSMA3 | 1.239 | 0.719 | −0.053 | 0.001 | 0.093 | 1.824 | 2.179 | 0.244 | −0.001 | 0.788 | 0.158 | 0.235 | −0.313 | −0.388 | 0.005 |
| PSMA7 | −0.689 | −0.075 | 0.298 | 0.908 | 0.417 | 0.223 | −0.172 | −0.599 | −0.348 | −0.46 | −1.103 | −1.041 | −1.012 | −0.567 | −0.092 |
| PSMC5 | 2.8215 | 0.3445 | 3.0805 | −0.1615 | 0.3955 | −0.0285 | −0.0815 | −0.1275 | −0.0635 | −0.1635 | −0.8176 | −0.2985 | −0.6475 | −0.8355 | 1.0635 |
| PSMC6 | 1.87 | 0.923 | 3.274 | 0.626 | 0.675 | 0.31 | 0.713 | −0.254 | −0.158 | −0.002 | 0.002 | −0.655 | −1.018 | 0.074 | 0.399 |
| PSMD12 | 2.9205 | 1.1775 | 1.8825 | 0.8035 | −0.4495 | 3.7985 | −0.3485 | 0.3155 | −0.4895 | 1.2245 | −0.4485 | 0.5695 | 0.5085 | −0.0055 | 0.2675 |
| PSMD12 | 1.2155 | 1.7605 | −1.2435 | 0.0105 | 0.6955 | 1.0145 | 1.1425 | −1.4735 | 0.2845 | −0.7885 | 1.0895 | −2.0025 | 0.0145 | −0.2685 | −0.3455 |
| PSMD3 | −0.3645 | 0.3865 | −0.2965 | −0.1105 | −0.1895 | 0.4835 | −0.6255 | −0.5135 | 0.2705 | 0.0245 | 0.9375 | −0.0565 | −0.0235 | 0.1455 | 0.1185 |
| PSPH | −0.723 | −0.366 | −0.692 | −0.494 | −0.533 | −0.127 | 1.774 | 0.249 | 0.664 | 0.792 | −0.653 | 0.865 | −0.549 | −0.765 | −1.601 |
| PTD012 | −0.007 | −0.134 | 0.019 | 0.126 | −0.158 | −0.434 | 0.113 | −0.356 | −0.1 | −0.081 | −0.232 | −0.313 | −0.295 | 0.337 | −0.01 |
| PTGER3 | 1.443 | −0.318 | −0.312 | 0.54 | 0.108 | −0.01 | 0.257 | −0.571 | −0.044 | 0.188 | −0.159 | −1.002 | −0.581 | −0.888 | −0.625 |
| PTHLH | −0.7115 | −0.2275 | −0.3235 | −0.8775 | −0.1225 | 0.2805 | −0.2425 | 0.6515 | −0.3635 | −0.0965 | 0.3285 | 0.4885 | 0.0225 | −0.0655 | 0.4455 |
| PTK6 | 0.785 | −0.178 | 0.23 | 0.184 | 0.599 | 1.328 | 0.78 | 1.378 | 0.315 | 0.073 | 0.369 | 0.462 | 0.709 | −0.381 | 0.189 |
| PTPN4 | 0.44 | −0.699 | −0.166 | −0.865 | −0.4 | −0.357 | 0.9 | −0.65 | −0.019 | −0.188 | 0.34 | 1.94 | 0.944 | 1.079 | 0.503 |
| PTPN7 | −0.172 | −0.156 | −0.102 | −0.235 | 0.511 | −0.227 | −0.991 | 0.169 | 0.21 | 0.155 | −0.277 | −0.568 | −0.084 | 0.965 | −0.729 |
| PTPRF | −0.192 | −0.054 | −0.262 | −0.241 | −0.158 | −0.425 | −0.559 | 0.075 | 1.023 | −0.207 | −0.082 | −0.4 | −0.072 | −0.257 | −0.584 |
| PTPRN2 | −0.6125 | −0.3205 | 0.7875 | 0.6885 | 0.0135 | −0.3265 | 0.1835 | −0.4805 | −0.1995 | −0.3875 | 0.5815 | 0.3575 | 0.3905 | −0.0255 | 1.0335 |
| PTPRT | 1.3045 | −0.1855 | 0.2225 | 0.4985 | 0.3235 | −0.4105 | −0.4985 | −0.1995 | −0.3535 | −0.5085 | 2.1785 | 0.8015 | 0.0905 | 1.4185 | 0.8455 |
| PTS | −0.057 | −0.356 | 0.292 | −0.395 | 0.878 | 0.161 | −0.338 | −0.485 | −0.533 | −0.155 | −0.358 | −0.154 | −0.608 | 1.036 | 0.782 |
| PTTG1 | −0.4485 | −0.2675 | −0.4515 | −0.5845 | −0.0785 | −0.9415 | −0.6435 | −0.5555 | −0.0645 | −0.5615 | −0.1145 | 0.6085 | −0.8995 | 0.4015 | 0.5965 |
| PURB | 1.0485 | −0.7295 | −0.2615 | −1.6065 | −0.2505 | −0.8545 | 1.0335 | 1.6315 | 0.4875 | 1.5595 | −0.8775 | −0.1875 | −0.3755 | −1.9565 | −0.7575 |
| PXMP2 | 1.9155 | 0.0125 | −0.0765 | 0.8965 | 0.0175 | 0.8065 | −0.2435 | 0.0825 | 0.5515 | 0.1055 | 0.0215 | −0.3895 | 0.0585 | −0.2045 | −0.5635 |
| PYCARD | 0.65 | −0.547 | 0.024 | −0.221 | −1.019 | −0.689 | −0.957 | −0.284 | −1.168 | 0.115 | −1.322 | −0.88 | −0.613 | −0.742 | 0.002 |
| PYCR1 | 2.382 | 1.116 | 0.976 | 0.745 | 0.716 | 1.852 | 0.171 | 0.203 | 1.067 | 0.496 | 0.243 | −0.804 | 0.056 | 0.517 | −1.106 |
| QDPR | −0.5265 | −0.6735 | −0.2495 | −0.8785 | −0.0535 | −0.9555 | −0.3845 | 0.1795 | −0.4095 | 0.5455 | −0.5625 | 0.1285 | 0.4745 | −0.5795 | 0.3535 |
| QPRT | −1.172 | −2.179 | −1.146 | −0.898 | −0.158 | −0.263 | 0.54 | 1.533 | −0.813 | 0.507 | −1.907 | −1.874 | −1.363 | −0.67 | −1.889 |
| QSCN9 | 0.8175 | −1.3875 | −0.0805 | 0.9365 | 1.3025 | 0.0005 | 0.3525 | 0.5045 | 1.0025 | 2.1075 | −0.7585 | −0.6875 | 0.3545 | 0.2375 | 0.0495 |
| RAB11FIP | −0.133 | −0.197 | −0.281 | −0.075 | 0.016 | −0.443 | 0.11 | 0.001 | 0.083 | 0.595 | 0.121 | 0.136 | 0.359 | 0.296 | −1.245 |
| RAB11FIP | 0.269 | −0.124 | 0.88 | 0.487 | 0.175 | 0.744 | 1.303 | 0.088 | −0.734 | −0.308 | 0.999 | 0.12 | −0.514 | −0.171 | 0.126 |
| RAB13 | −0.395 | −0.349 | −0.26 | −0.345 | −0.09 | 0.373 | −0.28 | 0.144 | 0.095 | −0.334 | −0.226 | −0.632 | −0.241 | 0.709 | −0.47 |
| RAB27A | −0.872 | −1.237 | −0.412 | −0.973 | −0.433 | −0.964 | 0.273 | −0.818 | 0.081 | 1.365 | 0.022 | 0.248 | 0.217 | −0.205 | 0.085 |
| RAB30 | 0.9755 | −0.2105 | −0.0045 | 0.5815 | 0.3505 | −0.0685 | 0.7475 | −0.1055 | 0.0385 | 0.4515 | 0.7675 | 0.5285 | −0.3715 | 0.2225 | 0.9485 |
| RAB34 | 0.06 | −0.091 | 0.217 | 0.108 | 0.679 | 0.399 | −0.516 | 0.958 | −0.161 | 0.006 | 0.371 | −0.607 | −0.32 | −0.006 | 0.235 |
| RAB35 | 0.304 | 0.041 | −0.075 | −0.064 | 0.038 | −0.192 | −0.079 | −0.019 | −0.277 | −0.913 | −0.041 | 0.227 | −0.435 | −0.041 | 0.399 |
| RAB39B | 1.9245 | −0.4835 | 0.1365 | 0.3425 | 0.2845 | −0.7755 | 0.3905 | −0.1495 | 0.3455 | −0.5435 | 0.4555 | −0.0915 | 3.6985 | 0.0105 | −0.5415 |
| RAB3A | −0.701 | −0.825 | −0.54 | −0.301 | 1.189 | −0.058 | −2.329 | 0.249 | 1.833 | 2.468 | −1.115 | 0.697 | −0.009 | 1.315 | 0.77 |
| RAB3IP | 0.268 | −0.327 | 0.015 | −0.354 | −0.291 | 0.824 | −0.001 | 0.99 | −0.692 | −0.214 | −0.028 | 0.692 | 0.072 | 0.033 | −0.081 |
| RABEP1 | −0.138 | 0.42 | 0.596 | 0.695 | 0.824 | 0.026 | −0.473 | −0.026 | −0.358 | −0.554 | 1.076 | 0.37 | −0.681 | 0.656 | 0.731 |
| RABGAP1 | −0.7695 | −0.3045 | −0.4765 | −0.4835 | 0.0275 | −0.0035 | −0.4135 | −0.1075 | −0.6225 | −0.4625 | −0.7125 | −0.1315 | −1.0455 | −0.5165 | −0.1955 |
| RACGAP1 | −0.0355 | 0.6215 | −0.2065 | −0.3245 | 0.2465 | 0.4535 | 0.5535 | −0.4955 | 0.6875 | 0.3255 | 0.9785 | 0.2145 | −0.4065 | 0.0595 | 1.1375 |
| RAD17 | −0.135 | 0.02 | −0.141 | 1.82 | 0.721 | 0.088 | −0.152 | −0.287 | −0.225 | 0.037 | 1.424 | 2.031 | −0.367 | 1.336 | 1.236 |
| RAD51 | 0.173 | −0.095 | −0.016 | −0.209 | 0.091 | 0.029 | −0.049 | 0.074 | 0.11 | 0.342 | −0.069 | 1.925 | 0.255 | 0.298 | 0.731 |
| RAD51AP | 0.7905 | −0.0005 | −0.4955 | −0.8515 | −0.8825 | −0.3205 | −0.5575 | 0.1775 | 0.2735 | −0.3885 | 1.1655 | 1.0885 | 0.1325 | 1.8255 | 1.4395 |
| RAD52 | −0.095 | 0.47 | 0.098 | −0.184 | 0.414 | −1.357 | −0.544 | −0.464 | −0.556 | −0.494 | 0.079 | 0.554 | 0.004 | 0.094 | 0.144 |
| RAD54L | 0.0505 | 0.1915 | 0.1775 | 0.5875 | −0.2875 | −0.0555 | 0.4585 | 0.3905 | −0.1515 | −0.2735 | 0.9835 | 0.8175 | 0.5505 | 1.0685 | 1.8145 |
| RAMP | −1.7225 | −1.4525 | −0.7995 | 1.6185 | −1.0185 | 1.8465 | 3.3925 | 1.7985 | −1.5385 | −0.3535 | 0.7615 | 0.7815 | 2.5335 | 2.7945 | 1.4045 |
| RANBP1 | −0.1485 | −0.3525 | 0.2645 | 0.2315 | 0.5905 | −0.0905 | 0.2345 | 0.0415 | 0.0795 | −0.7975 | 0.7525 | 0.0995 | −0.9585 | −0.1825 | 0.7265 |
| RARRES1 | −1.47 | −1.807 | −1.367 | −0.733 | −1.288 | −1.318 | −0.745 | −0.688 | 0.312 | −1.178 | −1.05 | −0.503 | 0.496 | −1.569 | 0.339 |
| RBBP7 | 0.1515 | 0.3845 | 0.2405 | 0.1555 | 0.0375 | −0.0965 | −0.0616 | −0.0285 | 0.1205 | −0.1715 | 0.8395 | 0.2795 | −0.0215 | 1.3355 | 0.7995 |
| RBBP8 | −0.1655 | 0.3075 | 0.1955 | 0.6505 | −0.0745 | −0.3105 | 0.1875 | −0.1025 | −0.2295 | −0.8375 | 0.0075 | 0.2745 | −0.5625 | −0.0875 | −0.0855 |
| RBM8A | 1.803 | −0.177 | 2.259 | 2.317 | −0.561 | 1.082 | −0.221 | −0.092 | 0.269 | −0.621 | 0.158 | 0.001 | −0.349 | −0.374 | −0.164 |
| RBX1 | −0.097 | 0.213 | −0.518 | −0.205 | −0.114 | 0.004 | −0.006 | −0.096 | 0.16 | −0.291 | 0.677 | 1.229 | −0.335 | 1.284 | 2.261 |
| RDHE2 | −0.162 | 0.264 | 0.016 | 0.009 | 0.36 | −0.264 | 0.009 | −0.032 | −0.08 | −0.03 | −1.332 | −0.287 | −1.002 | −0.701 | 0.485 |
| REA | 0.081 | 0.201 | 0.137 | 0.107 | 0.408 | 0.022 | 0.576 | −0.087 | 0.183 | 0.189 | 0.309 | −0.049 | 0.254 | 0.017 | 0.009 |
| REC14 | −0.1285 | −1.0455 | −0.2985 | −0.6835 | −0.4245 | 0.6305 | 0.5365 | 0.8075 | −0.1475 | 0.0495 | 0.3435 | 1.5045 | 0.7235 | 0.4785 | 0.6985 |
| REG1A | 0.266 | 0.043 | 0.025 | 0.19 | −0.132 | 0.462 | 0.001 | 0.145 | 0.274 | −0.111 | −0.283 | −0.499 | −0.13 | 0.067 | −0.125 |
| RELB | −0.857 | −0.606 | −0.748 | −0.194 | −0.004 | −0.408 | −1.523 | −0.292 | 0.201 | −0.607 | −0.686 | −0.121 | −0.638 | −0.658 | −0.455 |
| REPIN1 | −0.691 | −0.938 | −1.134 | −1.146 | −0.549 | −0.701 | −0.248 | −0.002 | 0.618 | −0.809 | −0.93 | −0.239 | −0.031 | 1.415 | 0.494 |
| REPS2 | −0.4005 | −0.2745 | 0.0055 | 0.5805 | −0.0025 | −0.0905 | −0.0655 | 0.3915 | −0.5285 | −0.2765 | 0.6005 | 0.8105 | −0.0795 | 1.4855 | 0.0275 |
| RERG | 0.2095 | 0.6645 | −0.5325 | −0.3125 | −0.2665 | 0.2885 | 0.0645 | 0.6265 | −0.3095 | −0.1965 | 0.2545 | 0.1795 | 0.4185 | −0.6455 | −1.1615 |
| RFC4 | 0.5325 | −0.4805 | 0.5405 | 0.7645 | −0.2715 | 0.3615 | 1.6685 | −0.1055 | 0.0175 | 0.4025 | 0.5855 | 0.9615 | −0.1085 | 0.9535 | 0.8965 |
| RGS10 | 0.2255 | −0.3085 | −0.1555 | −0.1055 | 0.2215 | −0.6995 | −0.3195 | −0.8645 | −0.6365 | −0.2725 | −0.1485 | 0.0695 | −0.5085 | −0.3895 | −1.1485 |
| RH8DL2 | 0.574 | 0.262 | 0.496 | −0.324 | 0.27 | −0.427 | −0.491 | −0.263 | −0.238 | 0.626 | −0.422 | −0.449 | −0.054 | −0.307 | 0.867 |
| RIBC2 | 0.404 | 1.165 | 1.115 | 0.662 | 1.24 | 0.451 | 0.285 | 0.082 | −0.194 | 1.181 | 0.898 | 0.435 | 0.588 | 0.542 | 0.401 |
| RIC-8 | −0.944 | 0.072 | −0.885 | −0.852 | −0.077 | −0.385 | −0.344 | −0.551 | −0.089 | −0.044 | −0.501 | −0.759 | −0.795 | 0.458 | −0.285 |
| RIT1 | −0.085 | −0.223 | −0.605 | 0.164 | −0.138 | −0.033 | 0.343 | 0.089 | 0.175 | −0.038 | 0.158 | 0.108 | −0.281 | 0.191 | −0.092 |
| RLN2 | 0.158 | −0.813 | −0.713 | 0.229 | −0.658 | −0.319 | 0.077 | 0.236 | 0.17 | 0.217 | −1.766 | −0.389 | −0.058 | −0.902 | −0.173 |
| RNF10 | −0.2215 | −0.7855 | −0.0335 | 0.7025 | −0.3985 | −0.2185 | 0.3475 | 0.2795 | −0.1955 | −0.3785 | 0.5835 | −0.0605 | −0.3895 | 0.4375 | −0.4815 |
| RNF135 | 0.1095 | −0.2935 | −0.1865 | −0.0595 | 0.8795 | −0.8785 | −0.9155 | −0.1305 | 1.0825 | −0.0985 | 3.6025 | 3.6915 | 2.3045 | 1.4655 | 1.2005 |
| RNF7 | 0.484 | 0.304 | −0.029 | 0.017 | 0.068 | −0.354 | 0.22 | −0.494 | −0.371 | −0.386 | −0.445 | −0.692 | −0.72 | 0.069 | 0.293 |
| ROPN1 | −0.7905 | −1.0475 | 2.5585 | −0.5925 | 1.5295 | −0.7745 | −1.3965 | −0.6795 | −0.0985 | −0.7985 | 0.0735 | −1.3055 | 2.9705 | 1.8325 | 0.7815 |
| RPA3 | −0.4575 | 0.1035 | −0.2405 | −0.4175 | −0.1545 | −0.0395 | −0.3905 | 0.4255 | −0.1965 | −0.2485 | −0.5055 | 0.1335 | −0.3085 | 0.0795 | −0.2015 |
| RPESP | −0.059 | 0.673 | −0.278 | −0.51 | 0.172 | −0.422 | 0.182 | 0.411 | 0.279 | 0.709 | 0.711 | 1.184 | −0.304 | 1.31 | −0.295 |
| RPL11 | −0.425 | −0.244 | −0.55 | −0.374 | −0.755 | −0.315 | −0.565 | 0.318 | 0.647 | 0.057 | −0.221 | 0.414 | −0.346 | 0.479 | −0.576 |
| RPL13 | −0.937 | 0.696 | −0.624 | 0.394 | 0.014 | −0.441 | −0.93 | −0.414 | −0.329 | −0.419 | −0.859 | −0.567 | −0.067 | −0.826 | 0.052 |
| RPL13A | −0.2655 | 0.5445 | −0.6665 | −0.1075 | −0.6605 | −0.0355 | −0.3925 | 0.2165 | −0.0485 | 0.7205 | −0.3085 | 0.1335 | −0.1825 | 0.0945 | 0.3495 |
| RPL15 | −0.478 | 0.685 | 2.385 | 0.824 | 1.538 | 1.359 | 1.256 | 0.279 | −0.068 | 0.382 | −0.381 | 0.846 | 0.334 | −0.327 | −0.536 |
| RPL18A | −0.73 | 0.837 | −0.159 | 0.517 | 0.171 | 0.096 | −0.426 | 0.183 | −0.013 | −0.436 | −0.109 | 0.31 | −0.484 | −0.485 | 0.185 |
| RPL19 | −0.11 | 0.611 | −0.525 | 0.091 | −0.423 | 0.155 | −0.163 | 0.272 | −0.358 | 0.445 | −0.126 | 0.214 | −0.292 | −0.779 | −0.738 |
| RPL21 | −0.2945 | 0.3205 | −0.5965 | −0.1095 | −0.5955 | 0.0995 | −0.3155 | 0.2535 | −0.2805 | 0.5025 | −0.3595 | 0.1575 | −0.2265 | −0.7275 | −0.7445 |
| RPL23A | −1.153 | 0.219 | −0.587 | −0.31 | −0.345 | −0.244 | −0.362 | 0.191 | −0.303 | 0.358 | 0.148 | 0.447 | −0.851 | 0.058 | −0.676 |
| RPL23A | −0.1715 | 0.0235 | −0.6445 | 0.0815 | −0.7745 | −0.0885 | −0.3015 | −0.3585 | 0.1315 | −0.4515 | 0.0285 | −0.0175 | −0.4195 | −0.0825 | −0.6025 |
| RPL26L1 | −0.2475 | 0.4735 | −0.5395 | −0.3515 | −0.8135 | −0.4155 | −0.7585 | 0.4675 | −0.1695 | 0.2025 | −0.0065 | 0.6425 | 0.6495 | 0.1235 | −1.0535 |
| RPL29 | −0.0115 | 0.3505 | −0.3935 | −0.1155 | −0.5255 | −0.0115 | 0.1285 | 0.0215 | −0.2515 | −0.2595 | −0.7605 | −0.1765 | −0.1405 | −0.0455 | −0.5915 |
| RPL35 | 0.0755 | 0.6865 | −0.1625 | −0.0155 | −0.6605 | −0.4345 | 0.2465 | 0.3665 | 1.3355 | 0.1755 | 0.7365 | 0.7875 | −0.7615 | 0.9985 | −0.4605 |
| RPL36AL | 0.8925 | 0.7555 | 0.6595 | 0.6055 | 0.5085 | −0.3335 | −0.4875 | −0.3395 | 0.5925 | 0.4915 | 0.9835 | −1.0775 | 0.8775 | 0.0765 | 0.0515 |
| RPS16 | −0.9505 | 0.0585 | 0.0735 | −0.4915 | −0.2215 | −0.1565 | −0.1965 | −0.1545 | −0.7815 | −0.3745 | −0.1915 | −0.4835 | −0.2595 | 0.2495 | −0.4215 |
| RPS4Y1 | 1.9285 | 0.2875 | 0.0705 | 1.0005 | 0.2795 | 0.5935 | 1.2335 | 0.0035 | −0.7325 | 0.1375 | 1.4515 | 0.0505 | 0.6155 | 1.2865 | −0.6055 |
| RRAGA | 0 | 0.541 | −0.149 | −0.405 | 0.304 | −1.013 | −0.726 | −0.27 | −0.672 | −0.09 | −0.337 | −0.205 | −0.34 | 0.112 | 1.317 |
| RRAGD | 1.74 | 1.42 | 0.5 | 1.475 | 0.797 | 0.464 | 0.511 | −0.006 | −0.619 | −0.403 | 1.167 | −0.055 | −0.748 | 1.39 | 0.27 |
| RRM1 | 0.311 | 0.065 | −0.07 | 0.091 | −0.334 | 0.008 | 0.7 | 0.009 | −0.194 | −0.184 | −0.306 | 0.176 | −0.581 | −0.035 | 0.264 |
| RRM2 | −0.447 | 0.56 | −0.578 | −0.888 | 0.102 | 0.341 | 0.126 | 0.242 | −0.752 | −0.583 | −0.094 | 0.833 | 0.884 | −0.457 | 1.247 |
| RSAFD1 | −0.0265 | 0.4695 | 0.4375 | 0.7475 | 0.0185 | 0.0935 | 0.8845 | 0.1095 | 0.3595 | 1.3605 | 0.5815 | 1.9435 | 0.6865 | 0.5375 | 0.4705 |
| RSNL2 | −0.3845 | −0.0925 | 0.0335 | −0.2835 | 0.1575 | 0.4365 | 0.4825 | 0.3745 | 0.0705 | 0.7615 | 0.1895 | 0.5765 | −0.7865 | 0.6575 | 1.3985 |
| RSU1 | 0.215 | 0.316 | 0.457 | 0.956 | 0.624 | 0.436 | 1.449 | 0.278 | 0.005 | 0.868 | 0.406 | −0.123 | 0.557 | 0.078 | −0.264 |
| RTN4IP1 | −2.192 | 2.599 | 0.41 | 1.524 | −0.928 | 0.541 | 1.648 | 1.878 | 0.307 | 1.004 | 0.969 | 1.279 | −1.044 | −0.428 | −0.196 |
| S100A11 | −2.068 | 1.856 | 0.499 | 0.832 | −0.195 | 0.825 | 0.658 | 0 | −0.025 | 0.531 | −0.928 | 0.66 | −0.569 | 0.46 | 0.022 |
| S100A14 | 3.5225 | 4.2355 | 3.8895 | 4.1255 | 1.0375 | 3.1655 | 4.3925 | 0.3005 | −0.9835 | 1.6315 | 3.4835 | 1.1415 | −0.6255 | 2.8845 | 2.0445 |
| S100A16 | 0.5945 | −0.1595 | 0.2275 | 0.3425 | −0.1485 | 0.2465 | −0.3175 | 0.1325 | −0.0345 | 0.3415 | 0.5975 | 0.2225 | −0.3665 | 0.9275 | 0.0125 |
| S100A9 | −0.88 | 0.287 | 0.226 | −0.707 | 0.415 | −0.078 | −0.185 | −0.656 | 0.281 | −0.185 | −0.675 | −0.499 | −0.232 | −0.168 | −0.478 |
| SAE1 | 0.2105 | −0.1815 | −0.4555 | −0.3115 | −0.5335 | −0.0465 | 0.0475 | −0.5475 | −0.3875 | −0.2495 | 0.0585 | −0.3705 | −0.4195 | 0.0005 | −0.0695 |
| SALL2 | 0.321 | −0.349 | −0.008 | 0.76 | −0.046 | 0.188 | −0.335 | −1.143 | −0.503 | −0.455 | −0.725 | −0.726 | −0.872 | −0.777 | −0.835 |
| SARA1 | 0.4075 | 0.0015 | −0.0405 | −0.2505 | 0.2315 | −0.1705 | −0.6155 | −0.0275 | −0.0385 | −0.2945 | −0.5055 | −0.8085 | −0.1665 | −0.7115 | −1.1415 |
| SARA2 | −0.283 | −0.457 | 0.447 | 0.437 | −0.327 | −0.692 | 0.655 | −0.083 | 0.118 | 0.642 | 0.446 | 1.038 | 0.715 | 0.77 | 0.369 |
| SAS | −1.145 | −0.966 | −0.877 | −0.635 | −0.769 | 0.463 | −1.164 | 1.052 | 0.652 | 0.393 | 0.012 | −1.023 | −0.913 | −0.778 | −0.288 |
| SCIN | 0.5195 | −0.1645 | −0.4795 | 0.0125 | −0.4185 | 0.3025 | 1.6195 | 0.6265 | −0.4845 | −0.1615 | −0.0125 | −0.0205 | 0.1685 | −0.2995 | −0.5845 |
| SCML1 | −0.623 | −1.543 | −0.509 | −0.062 | −0.725 | 0.119 | −0.36 | 0.196 | 0.035 | 0.604 | 0.324 | 1.511 | 1.448 | 0.575 | 0.595 |
| SCPEP1 | 0.9495 | 0.8895 | 1.3085 | 1.5535 | 1.5235 | 1.8135 | 2.1395 | 0.2925 | −0.3105 | 0.7535 | 0.6435 | 0.4515 | 0.8265 | 1.6165 | 1.4795 |
| SCRIB | 0.27 | −1.288 | 0.286 | −0.496 | −0.61 | 0.467 | 0.62 | 0.868 | −0.845 | 0.912 | 0.421 | 0.313 | 0.812 | 0.287 | 0.573 |
| SDC1 | −0.122 | 0.642 | 0.047 | 0.059 | 0.081 | −0.112 | −0.199 | −0.151 | −0.015 | 0.485 | 0.128 | 0.585 | 0.479 | −0.078 | 0.101 |
| SDC3 | 0.1845 | −0.0645 | −0.0275 | 2.1395 | −0.1015 | 0.1345 | 1.0095 | −0.1645 | −0.0945 | 0.7325 | −0.9205 | −0.2285 | 0.0365 | 0.3975 | 1.1585 |
| SDCCAG0 | 0.8335 | 0.8225 | 0.0955 | −0.0745 | −0.1745 | 0.2485 | 0.7385 | 0.4985 | 0.2155 | 0.9935 | 1.0145 | 0.2655 | 0.5085 | −0.1225 | −0.2475 |
| SEC61G | −0.342 | −0.037 | −0.344 | −0.779 | 0.557 | 0.02 | −0.26 | −0.103 | −0.999 | −0.348 | −0.5 | −0.21 | −1.08 | 0.05 | −0.09 |
| SECP43 | −0.5825 | −0.9895 | −0.7875 | −0.7865 | −0.9055 | −1.0265 | −0.5025 | −0.5575 | −0.3845 | 0.5915 | −0.1965 | −0.7105 | −0.6095 | −0.0065 | −1.0975 |
| SEDLP | −1.877 | −0.861 | −0.738 | 0.872 | 0.727 | −0.025 | 1.035 | −1.096 | −0.257 | −1.003 | −2.655 | −2.5 | −2.651 | −1.149 | −1.826 |
| SEMA3B | −0.4665 | −0.6495 | 0.5575 | 0.2595 | −0.9645 | 0.2245 | −0.1745 | 0.3295 | −0.5115 | 0.5515 | 0.4205 | 0.4015 | 0.6385 | 0.4355 | −1.2865 |
| SEMA3C | −0.7995 | −1.7715 | −0.6915 | −0.3225 | −0.6525 | 0.0865 | −0.0655 | 0.2775 | −0.3915 | −0.2535 | −1.2585 | −0.7115 | −0.3465 | −0.4805 | −0.4985 |
| SEPW1 | −1.329 | −1.314 | −1.088 | −1.019 | −1.355 | −1.388 | −1.514 | −0.991 | 1.023 | −1.179 | −0.971 | −0.112 | −1.08 | −0.752 | −1.364 |
| SFRP1 | −0.0655 | 0.1625 | 0.2105 | 1.5575 | 0.0985 | −0.4145 | −1.5995 | −0.7705 | 0.0075 | 0.0045 | 2.6335 | 0.9555 | 0.0335 | 0.1995 | 2.6965 |
| SERPINA [symbol] | −2.535 | −1.372 | −1.309 | −1.46 | −2.617 | 0.88 | −1.18 | 1.454 | 0.526 | −1.612 | 2.258 | 2.855 | 3.872 | −0.331 | 0.879 |
| SERPINB [symbol] | 0.36 | −0.214 | −0.223 | −0.404 | 0.078 | 0.429 | −1.22 | 0.083 | 0.174 | −0.004 | 0.179 | −0.083 | −0.527 | 0.273 | −0.2 |
| SFRP1 | −0.048 | 0.628 | 0.795 | 0.731 | 0.129 | −0.236 | 0.293 | 0.053 | −0.803 | 0.178 | −0.607 | 0.374 | −0.118 | 1.236 | −0.417 |
| SFRS6 | −1.8265 | −0.3585 | 0.1415 | 0.6005 | −0.0715 | −0.0185 | 1.3535 | −0.3865 | −0.9525 | −0.4045 | 0.8465 | 0.8405 | −0.1625 | −0.2255 | −0.4925 |
| SGCD | −1.0725 | 0.0395 | −0.0285 | 0.0885 | 1.3385 | −0.4185 | −1.1516 | 0.3695 | 0.0265 | −0.8815 | −1.2415 | −1.0685 | −1.4085 | −2.6085 | −1.1585 |
| SGCE | 0.961 | 0.173 | −0.3 | 0.955 | 0.256 | −0.135 | 0.902 | −0.044 | 0.127 | 0.268 | 0.192 | −0.165 | 0.122 | 0.487 | 0.888 |
| SH3BGRL | −0.821 | 0.057 | 0.071 | −1.12 | −0.218 | 1.005 | −1.551 | 0.061 | −0.365 | −0.058 | −0.368 | −0.644 | −0.87 | −0.826 | −0.408 |
| SHFM1 | −0.0065 | 0.3105 | −0.5165 | −0.5365 | −0.2075 | 0.4175 | 0.5015 | 0.2475 | −0.1125 | 0.0525 | 0.0915 | 0.3675 | 0.3015 | −0.0905 | −0.8095 |
| SIAT7B | 0.019 | −0.48 | −0.822 | −0.72 | 0.388 | 1.502 | 1.068 | 2.408 | 0.437 | 0.308 | 0.333 | 0.251 | 0.968 | −0.858 | −0.373 |
| SIGLEC12 | −0.004 | −0.445 | −0.766 | −0.824 | −0.642 | −0.622 | 0.343 | 0.055 | −0.051 | 0.331 | −0.499 | 0.416 | −0.058 | 0.131 | 2.202 |
| SIT | 0.2035 | −0.6885 | −0.0785 | 0.1335 | 0.6635 | −0.6235 | −1.1235 | −0.6255 | −0.7845 | −0.7345 | −0.7485 | −0.6645 | −0.9995 | −0.7765 | −0.3045 |
| SITPEC | 0.7235 | −0.2915 | −0.5475 | −0.3315 | −0.2745 | −0.0125 | 1.1015 | 0.3505 | 0.5895 | 0.2085 | 0.0965 | 0.5545 | 0.5925 | −0.1145 | 0.4315 |
| SKP1A | 2.9755 | −0.0285 | 0.4315 | 0.1235 | −0.2035 | 1.0385 | −0.2715 | −0.0415 | 0.3035 | −0.3125 | 0.5745 | 0.1695 | 1.5055 | −0.0255 | 0.2085 |
| SLC12A9 | 1.455 | 1.015 | 0.263 | −1.758 | −0.434 | −0.509 | 0.441 | 0.294 | −0.327 | −0.429 | −1.366 | −0.324 | −1.511 | −1.06 | −1.527 |
| SLC13A3 | −0.2285 | −1.2875 | −0.7495 | −0.8175 | −1.3745 | −0.3645 | −1.2025 | −0.1925 | 0.1585 | −1.4955 | −2.6955 | −1.1185 | −1.4165 | −0.8325 | −1.1845 |
| SLC16A6 | 0.215 | 0.172 | 0.186 | −0.443 | −0.254 | 0.089 | 0.944 | −1.472 | −0.238 | −0.356 | −0.972 | −1.876 | −0.337 | 0.597 | −0.896 |
| SLC19A2 | 0.5125 | −1.2145 | −0.7035 | 0.9125 | 0.0925 | −0.6485 | −0.3655 | 0.0755 | 0.1965 | 0.6015 | −1.3075 | −2.0705 | −0.7025 | 0.0485 | −0.6435 |
| SLC1A1 | −0.4775 | 1.0095 | 1.0325 | −0.5065 | 1.4135 | 1.6065 | −0.1835 | 0.5855 | −0.1185 | 1.2295 | −0.4235 | 0.1585 | 1.5835 | −0.8245 | 0.7405 |
| SLC1A2 | 0.2495 | −0.1065 | −0.3585 | −0.5765 | 0.3115 | 0.0415 | −0.1195 | −0.1065 | 0.4185 | −0.3655 | −0.5335 | −0.3835 | 0.5035 | 0.3695 | 1.0425 |
| SLC24A3 | 0.734 | −0.233 | −0.142 | 0.822 | 0.512 | −0.281 | −0.291 | 0.815 | −0.869 | 0.295 | −0.031 | 0.34 | −1.629 | −1.124 | 0.142 |
| SLC25A29 | 0.665 | 1.092 | 0.211 | 0.135 | 0.804 | 0.795 | 0.383 | 0.388 | −0.57 | −0.98 | −0.623 | −0.071 | −0.635 | −0.312 | 0.445 |
| SLC25A4 | 1.538 | −1.129 | −0.73 | −1.553 | −0.941 | −0.496 | 0.139 | 0.929 | −1.011 | −0.833 | −0.638 | −0.767 | −1.464 | −0.769 | −1.27 |
| SLC25A5 | −0.3065 | 0.1625 | −1.1015 | −0.7635 | −1.6185 | −0.2195 | −0.9235 | −0.2525 | 0.0665 | −0.3505 | −0.2755 | 0.0545 | −0.0875 | 1.0855 | −0.2565 |
| SLC27A2 | 0.183 | 0.288 | 0.12 | 0.231 | 1.488 | −0.077 | −0.538 | 0.542 | 0.148 | 1.351 | 1.422 | −0.375 | 0.071 | 0.275 | 0.142 |
| SLC27A5 | 0.27 | −0.96 | 0.385 | 1.061 | −0.487 | 0.4 | 0.026 | 0.427 | 0.918 | 0.47 | 0.177 | 0.941 | 1.834 | 0.451 | −0.208 |
| SLC30A8 | −2.8695 | −1.8825 | −1.7145 | −2.2025 | −0.4345 | −1.9705 | −1.7305 | 0.3425 | −0.9865 | −0.9665 | −2.9085 | −0.5665 | −0.7885 | −3.2685 | −0.5485 |
| SLC39A4 | −1.35 | 0.022 | 0.362 | 0.528 | 0.126 | 0.207 | 1.118 | 0.402 | 2.146 | 0.183 | −3.228 | −1.299 | −1.12 | −2.303 | −2.804 |
| SLC39A6 | −0.1275 | −0.4425 | −0.5205 | 0.1405 | 1.2495 | −0.3605 | −0.2515 | −0.2865 | −0.0975 | 0.3205 | 0.7865 | 0.8785 | 0.7145 | 0.3495 | 0.0005 |
| SLC40A1 | 1.0195 | 0.9535 | 0.2205 | 0.4685 | 0.2945 | 1.4075 | 1.1945 | 0.4685 | 0.2395 | 0.8095 | −0.2285 | 0.2605 | 1.7035 | 1.8935 | 1.0705 |
| SLC41A3 | 0.254 | 0.31 | 0.083 | 0.107 | 0.031 | −0.264 | −0.462 | 1.91 | 0.531 | 0.163 | 0.365 | −0.21 | 0.131 | 0.279 | 0.044 |
| SLC5A6 | 0.1185 | 1.3625 | −0.3665 | −0.7765 | −0.2175 | 0.4345 | 0.7455 | 0.5975 | 0.1995 | 0.2865 | −1.8305 | −1.0085 | −1.6325 | −0.5145 | −0.7075 |
| SLC6A4 | −3.0425 | −2.5505 | −1.1215 | 0.9385 | 1.9775 | −3.6645 | 0.4485 | 2.1045 | −0.7635 | −1.4775 | −0.9395 | −1.1635 | 1.8345 | 1.4645 | −2.4235 |
| SLC9A3R1 | −1.0915 | 0.2805 | 0.6485 | 0.7125 | −0.1655 | −0.0285 | −0.4415 | 0.1335 | −0.6335 | −0.5205 | −0.0905 | −2.0455 | −1.6005 | −0.5975 | −0.9165 |
| SLITRK6 | −0.341 | 0.218 | 0.349 | 0.155 | 0.349 | 0.252 | 0.439 | −0.124 | −0.258 | 1.101 | 0.298 | 0.599 | −0.729 | 0.193 | 0.851 |
| SMA3 | −0.437 | 0.042 | 0.252 | −0.274 | 0.08 | 0.537 | 1.199 | 0.606 | 0.135 | 0.066 | 0.514 | 0.287 | 1.532 | 1.898 | 1.387 |
| SMAD2 | −0.039 | 0.228 | 0.088 | 0.1 | −0.155 | −0.208 | −0.688 | −0.088 | −0.412 | −0.349 | 0.349 | −0.591 | −0.077 | 1.378 | 0.703 |
| SMO | 0.197 | 0.092 | 0.149 | 0.102 | 0.085 | 0.753 | −0.301 | −0.193 | −0.2 | −0.004 | 0.207 | −0.308 | 0.079 | −0.189 | −0.082 |
| SMU1 | 0.635 | 0.324 | 0.012 | 0.178 | 0.210 | 0.767 | −0.099 | 0.069 | 0.237 | −0.1 | 0.415 | −0.088 | −0.056 | 0.056 | 0.588 |
| SNPH | 0.138 | 0.174 | 0.147 | −0.009 | 0.346 | −0.267 | −0.108 | 0.235 | −0.305 | −0.498 | 1.148 | 1.029 | 0.952 | 2.627 | 1.108 |
| SNRPB2 | 0.7495 | 0.0745 | 0.3295 | −0.1205 | −0.1205 | 0.1055 | −0.3835 | 0.1025 | −0.3355 | −0.5495 | 0.4585 | 0.3265 | 0.2325 | 0.3865 | −0.1955 |
| SNRPD1 | 0.43 | 0.315 | −0.019 | 0.953 | 0.592 | −0.181 | 0.23 | −0.282 | −0.607 | 0.011 | −0.079 | −0.252 | −0.421 | 0.727 | 0.52 |
| SNRPD3 | −1.0435 | −0.3045 | 0.0905 | 0.0545 | −1.1415 | −1.4645 | −1.3255 | −0.4815 | −0.1275 | 0.2595 | −0.8305 | 0.2855 | −0.9065 | −0.5245 | −0.3455 |
| SNRPG | 1.5695 | 0.1445 | 0.2455 | 1.0685 | 1.5385 | 0.7565 | 1.2035 | 1.3085 | −0.0995 | 1.7145 | 1.8695 | 0.8455 | 0.2565 | 0.2945 | 2.6865 |
| SNX7 | −0.1925 | −0.0245 | −0.2615 | −0.6215 | −0.8345 | −0.1765 | −0.5116 | −0.1575 | −0.0785 | 0.2065 | −0.3745 | −0.3565 | 0.0635 | −0.4475 | −0.4785 |
| SO02 | −0.5135 | 0.0495 | 0.4075 | −0.8035 | −0.3275 | 0.0505 | −0.0585 | 0.2385 | −0.1315 | 0.2315 | 0.9905 | 1.2125 | 0.9905 | 0.5745 | 1.0875 |
| SORCS1 | −1.184 | 0.621 | −0.571 | 0.46 | 0.03 | −0.921 | −0.852 | −0.442 | 0.401 | 0.8 | 0.272 | −0.755 | 0.119 | 1.148 | 0.422 |
| SOX4 | −0.0595 | −1.6095 | 0.1175 | −0.8005 | −0.1235 | −0.9495 | −0.5315 | −0.8875 | 0.6035 | −1.0455 | −0.4105 | −0.5845 | −0.0555 | −0.2035 | −0.2405 |
| SOX9 | 0.985 | 0.503 | −0.443 | 0.438 | 0.693 | 0.334 | 0.045 | 0.506 | −0.366 | 0.797 | 1.241 | 0.653 | 0.414 | 0.988 | 0.573 |
| SPAG18 | −0.5315 | 0.3995 | 0.4615 | 0.7075 | 0.3895 | 0.1205 | 0.7275 | −0.2625 | −0.1075 | 0.0295 | −0.5705 | −0.0415 | −0.5415 | −0.0545 | 0.5105 |
| SPAG5 | 0.4255 | −0.5655 | −0.5125 | −0.8485 | −0.0545 | −0.9285 | 0.0125 | 1.2135 | 0.3215 | 1.2065 | −0.0185 | 0.0035 | −0.4375 | 0.2405 | −0.3485 |
| SPC18 | −0.78 | −0.307 | 0.558 | −0.215 | −0.481 | −0.538 | 0.975 | −0.233 | 0.033 | 0.456 | −0.703 | 0.858 | −0.284 | 0.537 | 0.504 |
| SPFH2 | −0.489 | −0.622 | −0.418 | −0.378 | −0.098 | −0.192 | −0.267 | −0.244 | −0.122 | −0.628 | 0.742 | 0.705 | −0.347 | 0.436 | 1.709 |
| SPG3A | −0.125 | 1.505 | 1.475 | 0.855 | 1.945 | 0.485 | −0.505 | 0.255 | 1.147 | −0.595 | −0.195 | 1.035 | 1.495 | −0.344 | −0.638 |
| SPPL3 | 0.698 | 0.924 | 0.938 | 0.938 | −0.03 | 1.192 | 0.748 | 0.026 | −0.488 | 0.637 | −0.654 | −0.389 | −0.762 | −2.067 | −1.703 |
| SQLE | 1.944 | 0.579 | 0.308 | −0.155 | −0.826 | −0.072 | 0.042 | −0.635 | −0.033 | 0.279 | 0.539 | −1.539 | −1.704 | −1.084 | −2.022 |
| SQRDL | −1.0565 | −0.3525 | −0.6045 | −0.0025 | −0.4285 | −0.1315 | 0.0385 | −0.2235 | −0.2715 | −0.2725 | −0.8795 | −0.5605 | −0.5675 | −0.8505 | 0.1715 |
| SREBF1 | −0.1795 | 0.7665 | 0.5445 | 0.1845 | 0.9225 | 0.8965 | −0.4605 | 0.4145 | 0.2325 | 0.1955 | 1.0805 | 0.2715 | 0.1825 | 0.6455 | 1.1305 |
| SRI | −1.3285 | −0.8775 | −0.4145 | −1.0525 | −0.4145 | −0.1925 | −0.3915 | −0.1855 | −0.1785 | −0.7315 | −1.1695 | 0.3605 | −0.5845 | −1.0585 | −0.3635 |
| SRPK1 | 0.6015 | 0.5685 | −0.2235 | 0.4085 | 0.7215 | 1.5935 | 1.0645 | −0.4125 | 0.3165 | −0.5035 | −0.7645 | −0.3925 | −0.2555 | −0.7265 | 0.0135 |
| SSFA2 | −0.6385 | −0.3815 | 0.3485 | 0.5675 | 0.4345 | −0.3385 | −0.3135 | −0.2805 | −0.4855 | −0.0665 | −1.2885 | −0.6265 | −0.6875 | −0.4585 | 0.0525 |
| SSR4 | 0.1805 | −0.6025 | −0.5185 | −0.7645 | −0.5015 | 0.3465 | 0.8175 | 0.0815 | −0.1805 | −0.2815 | 0.1785 | 0.2785 | −0.0945 | −0.6015 | 0.4885 |
| STARD3NI | −1.0175 | −0.3235 | 0.5225 | 0.3085 | 2.3655 | 1.3205 | 1.1385 | 1.2845 | −0.5725 | −0.6345 | 0.8845 | −0.2875 | −0.1625 | −0.6595 | −0.0885 |
| STARD7 | −1.3045 | −1.7505 | −1.5225 | 0.1185 | −1.8395 | −1.7145 | −1.9145 | −0.9615 | 1.9895 | 0.1985 | 0.0805 | 0.2525 | −0.6405 | −0.7985 | −0.0175 |
| STAT1 | −0.8325 | 0.0355 | −0.0715 | 0.8855 | 0.3835 | −0.1815 | 0.3725 | 0.0465 | −0.7855 | −0.9015 | 1.3905 | 0.2835 | −0.2915 | −0.2215 | 0.4075 |
| STC2 | 0.5635 | 0.3195 | 0.3635 | 0.5076 | 0.3225 | 0.4365 | 0.1105 | −0.0745 | 0.0755 | 0.0615 | 1.2305 | 0.0555 | −0.0015 | 1.0765 | 1.0425 |
| STK24 | 0.3175 | 0.1855 | 0.9025 | 0.6735 | 1.2065 | 0.4835 | −0.7495 | −0.4565 | −0.1755 | 0.4225 | 1.0675 | −0.0565 | −0.2085 | 1.1825 | 0.3955 |
| STK6 | 0.453 | 0.858 | 0.394 | 1.067 | −0.082 | 0 | 1.142 | −0.143 | 0.391 | 0.395 | 0.195 | −0.233 | 0.311 | 0.654 | 0.227 |
| STMN1 | 1.2885 | 0.4285 | 1.2115 | 2.0465 | 1.0585 | 0.7325 | 0.4575 | −0.1875 | 0.5195 | −0.0275 | 0.5455 | 0.2085 | 0.3145 | 0.8015 | 0.6955 |
| STX1A | 0.4215 | 1.0135 | 0.7455 | 0.9485 | 0.1835 | 0.6465 | 0.7585 | −0.7445 | −0.1935 | 0.9855 | −0.0905 | −0.8345 | −0.5825 | −0.0285 | 0.3325 |
| STX3A | −0.9045 | 0.5815 | −0.3335 | 0.4535 | −0.2265 | −0.4885 | 0.0035 | −0.0045 | −0.5805 | 0.1065 | 0.6715 | 0.6875 | 0.5405 | 0.2035 | 0.3465 |
| STYX | −0.3035 | 0.3255 | −0.0575 | 0.2865 | 0.2655 | −0.5515 | 0.1895 | 0.1425 | 0.0785 | −0.2215 | −0.7305 | −0.0755 | −0.0025 | −0.6545 | 0.1315 |
| SUGT1 | −1.092 | 1.997 | 0.181 | −0.325 | 0.328 | 0.301 | 0.667 | −0.03 | −0.385 | −0.557 | 0.245 | −0.336 | −0.536 | −0.102 | 0.118 |
| SUMF1 | −0.4285 | 0.3185 | 0.1955 | 1.0685 | −0.2835 | 0.0945 | 0.4815 | −0.4005 | 0.1795 | 0.7415 | −0.3965 | 0.3855 | −0.9475 | 1.7435 | 3.1065 |
| SUMO2 | 0.8885 | 0.9815 | 0.3995 | 0.6085 | 0.5645 | 0.0765 | 0.1465 | −0.7445 | 0.5305 | −0.4145 | 0.3515 | −1.2335 | −0.6525 | 0.3465 | 1.4775 |
| SUV39H2 | 0.1105 | −0.9365 | 0.4015 | −0.0095 | 1.4255 | 0.1215 | −0.3765 | 0.7715 | 0.4085 | −0.2885 | −0.1145 | 0.5355 | 0.5855 | 0.5335 | −2.5135 |
| SYAP1 | 0.3695 | 0.9375 | 0.4495 | 1.8075 | −0.2745 | 0.9065 | −0.5315 | 0.1835 | 0.4845 | 0.3585 | 0.7685 | −0.4555 | 0.9055 | 0.0695 | −0.5915 |
| SYCP2 | −0.077 | −1.243 | 0.027 | 0.851 | 0.535 | −0.438 | 1.683 | −0.052 | −0.227 | −1.17 | −1.243 | −0.513 | −1.523 | −1.575 | −2.5 |
| SYNGR1 | −1.4385 | −0.0785 | −0.6785 | 0.3905 | 0.3165 | 0.0045 | −0.3995 | −0.5595 | −0.4355 | −0.4185 | −0.7085 | −0.0485 | −0.0045 | −0.4095 | −0.5325 |
| SYTL2 | −0.1905 | −0.0515 | −0.3415 | 0.3635 | 0.5125 | −0.1855 | 0.8745 | −0.1385 | 0.0355 | 0.3725 | −0.0345 | 0.1685 | 0.0395 | 0.3135 | 0.2935 |
| TAF18 | −0.9235 | −0.2815 | −0.7885 | −0.8205 | −0.7435 | −0.1495 | 0.0895 | 0.5095 | 0.5505 | −0.0225 | 0.5785 | 0.2195 | 0.9855 | 0.0665 | 0.1095 |
| TAF8 | 0.327 | 0.328 | 0.638 | 1.063 | 0.856 | 1.215 | 0.218 | −0.177 | −0.201 | 0.477 | 1.204 | 0.187 | 0.257 | 0.712 | 0.209 |
| TARBP1 | −1.066 | 2.039 | 0.494 | 0.191 | −0.027 | −0.029 | −1.442 | −0.711 | −0.055 | −0.461 | 0.242 | 0.415 | 0.255 | −0.005 | 0.655 |
| TARS | −0.3275 | −0.1915 | −0.1825 | −0.2105 | 0.3395 | 0.4905 | −0.2035 | −0.0535 | −0.4795 | −0.2865 | −0.8345 | 0.2945 | −0.5405 | −1.2255 | −0.8375 |
| TATDN1 | 0.2205 | −0.4695 | −0.5615 | −0.8885 | 0.6385 | 0.0235 | −0.6375 | −0.1295 | −0.2545 | −0.1685 | −0.6385 | 0.0905 | 0.8165 | −0.2905 | 0.9085 |
| TBC1D12 | 2.9645 | −0.4015 | −0.0805 | 0.1725 | −0.1155 | 2.2445 | 2.2855 | 0.2145 | 1.2515 | 1.5095 | 1.0845 | 0.3935 | 1.9105 | 0.3485 | −0.5495 |
| TBC1D14 | 0.057 | −0.505 | −0.32 | −0.093 | 0.348 | −0.492 | −0.661 | −0.268 | −0.347 | −0.338 | −0.035 | −0.526 | −0.244 | 0.043 | 0.314 |
| TBC1D3 | −1.6925 | −0.7765 | 0.6395 | 1.3065 | −1.3115 | −1.3885 | 1.4345 | 1.9725 | 0.6365 | 1.1395 | −0.7785 | 1.2225 | 1.5925 | −2.0115 | 1.0455 |
| TBCA | −2.243 | −1.242 | −1.168 | −2.522 | 0.106 | −1.982 | −1.505 | −0.217 | 0.881 | −0.698 | −1.102 | −1.629 | −0.11 | −3.885 | −3.505 |
| TBX1 | −0.1235 | 1.9255 | 1.4535 | 0.4595 | 0.7815 | 1.8355 | 2.0535 | 0.0545 | −0.0375 | 1.2945 | −0.0485 | −0.0005 | 0.3275 | 0.1735 | 0.0005 |
| TBX3 | −0.302 | −0.022 | −0.004 | −0.487 | 0.062 | −0.46 | −0.845 | −0.539 | −0.395 | −0.404 | −0.481 | −0.83 | −0.56 | −1.088 | −0.898 |
| TCAP | −0.731 | 0.506 | 0.261 | −0.579 | 0.329 | −0.439 | −0.164 | −0.384 | −0.797 | −0.128 | −0.86 | −1.014 | −0.952 | −0.732 | −1.645 |
| TCEAL1 | −0.2955 | 0.0775 | −0.3235 | −0.8845 | 0.4015 | −0.0945 | −0.3565 | −0.2075 | −0.7375 | −0.4965 | −0.8205 | −0.3995 | −0.8515 | 0.0485 | −1.0045 |
| TCEAL3 | 0.878 | −0.721 | −0.161 | 0.67 | −0.244 | 0.442 | 0.128 | 0.66 | 1.448 | 1.813 | 0.726 | 0.827 | 1.68 | 0.359 | −0.347 |
| TCEAL4 | −0.066 | 0.317 | −0.206 | 0.263 | −0.234 | 0.656 | 0.536 | 0.318 | 0.047 | 0.827 | −0.041 | −0.297 | 0.16 | 1.152 | 0.31 |
| TCF15 | −1.175 | 0.802 | −0.121 | −0.449 | −0.071 | −0.452 | −0.174 | −0.469 | −0.372 | −0.438 | 0.321 | 1.006 | 0.036 | 0.705 | 0.182 |
| TCF3 | −0.516 | 0.119 | 0.245 | −0.274 | 0.298 | −0.134 | 0.214 | −0.001 | −0.373 | −0.188 | 0.463 | −0.38 | −0.306 | −0.565 | −0.253 |
| TCF7L2 | 1.1395 | −0.2355 | −0.1095 | −0.3055 | 0.1115 | −0.3005 | −0.4595 | 0.8205 | 0.7755 | 0.4225 | 0.3595 | −0.1315 | −0.5145 | 0.0085 | 0.2115 |
| TDRD7 | 1.0415 | 0.7225 | 0.7575 | −1.9235 | −0.1755 | 1.0895 | 1.3785 | 1.6255 | 0.8485 | 1.3975 | −3.9835 | −2.7455 | −3.4215 | 0.0505 | −1.5145 |
| TESK2 | −1.473 | −1.888 | −2.108 | −0.214 | −1.847 | −2.253 | −1.34 | −0.035 | 0.973 | −2.221 | 1.659 | 0.715 | −0.445 | 0.812 | 1.521 |
| TFAP2B | 0.129 | −0.216 | −0.367 | 0.933 | 0.204 | 0.468 | 1.538 | −1.077 | −0.189 | −0.2 | −0.185 | −0.169 | 1.355 | 0.659 | 0.524 |
| TFCP2L1 | 0.1305 | −0.8425 | −0.3435 | −0.6275 | −0.2385 | −0.5835 | −0.8965 | −0.3485 | 0.6345 | −0.6235 | 0.1555 | −1.1295 | −0.0075 | −0.6355 | −0.8735 |
| TFCP2L2 | 2.5485 | −1.5415 | −0.7635 | −1.3275 | −0.1375 | −1.0235 | −3.6485 | 0.2975 | 0.7415 | −0.6825 | −3.1555 | −2.8885 | −3.0255 | −4.0875 | −3.8965 |
| TFF1 | 0.5305 | −0.0195 | 0.5095 | 0.7695 | 0.8015 | 1.0345 | 1.5876 | 0.7585 | 0.2225 | −0.2885 | 0.3545 | −0.2385 | −0.3235 | 1.2815 | −0.1425 |
| TFF3 | −0.818 | −0.922 | −0.874 | −0.981 | −0.941 | −0.15 | −1.213 | 0.015 | 0.925 | −0.526 | 0.44 | 0.906 | −1.175 | −0.995 | −0.721 |
| TFRC | −0.7865 | −0.5905 | −0.2535 | −0.1595 | −0.2975 | 0.0495 | −0.6925 | −0.0755 | 0.3985 | −0.5785 | −0.2425 | 0.1735 | −0.3615 | 0.6405 | 0.2885 |
| TGFBR3 | 0.11 | −0.226 | −0.18 | −0.157 | −0.098 | 0.53 | 0.93 | −0.657 | −0.029 | 0.084 | −0.382 | −0.346 | −0.373 | 0.396 | 0.278 |
| THY28 | −1.5885 | 0.3815 | −1.0695 | −1.1385 | −0.8745 | 0.0675 | −1.1025 | 0.0145 | −0.2565 | −1.4555 | −0.9775 | −0.2375 | −1.0005 | −0.8265 | −0.8165 |
| TIF1 | −0.585 | −0.732 | −0.343 | −0.188 | −0.309 | −0.874 | −1.081 | −0.18 | −0.239 | −0.662 | −0.371 | −0.959 | −0.441 | −0.355 | −0.448 |
| TIGA1 | 0.098 | −0.538 | −0.059 | 0.368 | 0.41 | −0.408 | 0.391 | 0.273 | 0.393 | −0.305 | 0.034 | −0.043 | −0.293 | 0.816 | 0.299 |
| TIM14 | −0.2835 | 1.0585 | −0.1795 | 0.0055 | −0.5555 | −0.0945 | −0.0205 | −0.4255 | 0.0025 | −0.6415 | −0.4945 | −0.0665 | −0.3025 | −0.3185 | 0.3765 |
| TIMELES [symbol] | −1.1345 | −0.2045 | −0.5325 | −0.1625 | −0.2675 | −0.8305 | −0.8575 | −0.3595 | −0.7695 | −1.0595 | −0.5965 | −0.8355 | −1.1685 | −0.0345 | −0.4835 |
| TIMM9 | −1.57 | −1.024 | −0.681 | −0.963 | −0.203 | −0.542 | 0.766 | −0.383 | −0.383 | −0.718 | 1.313 | −0.765 | 0.036 | 1.201 | −0.871 |
| TINP1 | 5.0585 | 0.9515 | −0.0325 | 0.5985 | −0.6505 | 0.0695 | 0.8425 | −0.1975 | 0.0576 | 1.5985 | −0.2365 | −0.2015 | −1.3105 | 0.3235 | 0.1015 |
| [symbol] IP1208 | 0.474 | −0.290 | −0.678 | −1.078 | −0.165 | −0.363 | 0.52 | 0.073 | −0.01 | 0.519 | 0.378 | 0.116 | −0.321 | 0.618 | −1.801 |
| TK1 | −0.98 | −0.097 | −0.629 | −0.821 | −0.323 | −0.536 | 1.204 | 1.133 | −0.54 | 0.055 | −0.17 | 1.49 | 1.093 | −0.074 | −0.73 |
| TLE2 | −0.456 | −0.541 | −0.132 | −0.181 | 0.387 | 0.219 | −1.48 | −0.554 | −0.96 | −1.032 | −0.853 | 0.334 | 0.049 | −0.438 | 1.644 |
| TLR5 | −0.841 | −0.41 | 0.284 | 0.034 | 0.392 | 0.126 | −0.084 | 0.361 | −0.517 | −0.418 | −0.724 | −2.017 | −0.883 | −1.121 | −1.502 |
| TM4SF12 | −0.326 | −0.108 | 0.404 | 0.415 | 0.648 | −0.001 | 1.794 | −0.56 | −0.92 | −0.074 | −0.25 | −0.072 | −0.91 | −0.209 | −0.105 |
| TM4SF13 | −0.138 | −1.61 | 0.015 | −1.326 | −0.964 | −0.014 | −0.621 | −0.347 | 0.208 | −0.584 | −1.555 | −1.194 | −1.211 | −0.603 | −1.396 |
| TMEM14A | −0.3965 | −0.2385 | −0.1035 | −0.0295 | −0.2975 | −0.1875 | −0.0825 | −0.1355 | −0.3105 | −0.8245 | −0.5555 | −0.1815 | −0.2825 | 0.0915 | −0.2675 |
| TMEM25 | 0.4155 | 0.2755 | −0.0405 | −0.2025 | −0.3255 | −0.5835 | −1.1815 | 0.1745 | 0.3345 | −0.4845 | 0.3835 | 1.2855 | 1.9745 | −0.4085 | 3.4595 |
| TMEM4 | 1.781 | 0.249 | 1.507 | 1.996 | 1.625 | 0.999 | 2.024 | 0.421 | 0.131 | 1.004 | 1.217 | 0.803 | 0.311 | 0.913 | −0.597 |
| TMPRSS5 | 0.288 | 0.407 | 0.145 | 0.223 | 0.493 | 1.599 | −0.393 | −0.149 | −0.26 | 0.296 | 0.847 | −0.104 | 0.71 | 2.87 | 0.55 |
| TMSB10 | 0.297 | 0.418 | −0.117 | −0.459 | 2.271 | 3.572 | 1.82 | 0.033 | 0.691 | 0.133 | 0.213 | −1.829 | 0.022 | −1.274 | −1.236 |
| TMSNB | −0.0305 | 0.3085 | 0.7565 | 0.8135 | 0.6585 | 0.5485 | 1.3245 | 0.1165 | −0.5375 | 0.1365 | 0.2775 | −0.0525 | 0.6275 | 0.9455 | 0.5485 |
| TNFRSF1 [symbol] | 0.218 | 0.159 | 0.605 | 0.35 | 0.407 | 0.105 | 1.77 | 0.596 | −0.837 | 0.298 | 0.172 | 0.596 | 0.655 | 0.84 | 0.341 |
| TNFRSF2 [symbol] | 0.4755 | 0.6415 | 0.7205 | 1.5025 | 0.6225 | 0.0645 | 1.1145 | 0.8355 | 0.6885 | 1.0595 | 0.1815 | 0.2745 | 0.0235 | −1.1855 | −0.7585 |
| TNFRSF2 [symbol] | 3.3595 | −0.1965 | 2.3975 | 1.9675 | 0.3135 | 1.7475 | 1.9755 | 1.1645 | 1.5235 | −1.2635 | 0.9085 | −0.1255 | −0.9725 | 0.2385 | 0.6425 |
| TNFSF4 | −0.1875 | −0.8785 | 0.0205 | 0.1545 | 0.2035 | 0.0395 | 0.3895 | 0.1455 | −0.1485 | 0.0015 | −0.3795 | −0.7925 | 0.1735 | −0.2565 | 0.1955 |
| TNFSF7 | 0.0515 | −0.6825 | 0.3015 | 1.2585 | −0.2555 | −0.2765 | 0.7705 | −0.0475 | −0.5675 | 0.9735 | −0.1415 | −0.2125 | 1.3085 | 0.6905 | 0.3125 |
| TNIK | 0.9855 | −0.1165 | 0.6195 | −0.2035 | 0.0115 | 0.0805 | 0.8525 | 0.1405 | 0.5115 | 0.0485 | 2.7655 | 2.0585 | 0.1095 | 1.0335 | 0.0315 |
| TNNC2 | −0.412 | −0.473 | −0.098 | −0.353 | 1.98 | 0.767 | −1.359 | −0.747 | −0.473 | 0.266 | 0.589 | −1.154 | 0.17 | 0.304 | 0.001 |
| TNNI2 | 0.034 | 1.664 | −0.94 | −0.494 | −0.314 | 1.032 | −0.846 | 0.975 | −0.217 | −0.494 | −0.895 | −1.277 | −0.82 | −1.471 | 0.41 |
| TNNI3 | −1.3285 | 2.8585 | −0.5075 | 2.2035 | 1.1785 | −1.8035 | −0.3515 | −0.3265 | −0.6735 | −0.8405 | −0.8035 | −0.9755 | −1.4635 | −0.9385 | −1.2885 |
| TOB1 | −0.2025 | 0.0935 | −0.2355 | −0.5885 | −0.4985 | −0.3075 | −0.5885 | 0.2065 | −0.2305 | 0.9225 | −0.9495 | 0.0635 | −0.4285 | −0.3525 | −0.2625 |
| TOM1L1 | 0.1265 | −0.2805 | −0.4485 | 0.0575 | −1.0555 | −0.6965 | −0.1295 | 0.4525 | 0.2865 | 0.3405 | 0.9845 | 1.9695 | 1.9095 | 0.8405 | 1.0705 |
| TOMM7 | −0.8205 | −0.0995 | −0.4595 | −0.1405 | 0.5015 | 0.1465 | −0.5635 | 0.5915 | −0.7425 | 0.9225 | 1.1385 | 0.9375 | −0.3385 | 0.6515 | 0.9885 |
| TOP1MT | −0.812 | 0.438 | −0.124 | 0.334 | 0.184 | −0.108 | −0.254 | −0.338 | −0.395 | −0.183 | 0.049 | −0.665 | −0.749 | 0.349 | 1.239 |
| TOP2A | −0.7085 | −0.8225 | −0.3235 | 0.2095 | −0.2165 | −0.0935 | 0.8075 | 0.1055 | −0.2325 | −0.1675 | 0.4145 | 0.2865 | −0.0405 | 0.1555 | −0.1335 |
| TOP2B | −1.062 | 0.335 | −0.141 | −0.142 | 1.502 | −0.385 | −0.939 | −0.95 | −1.006 | −0.798 | 0.477 | −0.628 | −0.848 | 1.167 | 1.083 |
| TOPBP1 | 1.8175 | 0.5935 | −0.8855 | 0.6275 | −0.4565 | −0.4865 | 1.4995 | −0.0475 | 0.0895 | 0.2425 | −0.5915 | −0.5355 | −0.8445 | −0.9475 | 0.2525 |
| TOPK | −2.0705 | 0.1095 | 0.8205 | 0.4845 | 0.3925 | 0.3755 | −0.3245 | −0.4295 | −0.0235 | −0.0295 | 0.8725 | 0.0185 | 0.9995 | 0.8705 | 0.2355 |
| TP53AP1 | −1.0965 | −0.7265 | −0.1915 | −0.7525 | 1.8325 | 0.8345 | 0.1215 | 0.6045 | −0.3215 | 0.2215 | −0.6895 | −0.7835 | −0.3305 | −1.8125 | −1.5945 |
| TP53BP2 | −0.2805 | −0.1655 | 0.2635 | −0.1385 | −1.3555 | −0.3865 | −0.5055 | 0.7585 | 1.4695 | 1.0895 | −0.4745 | −0.8425 | −1.6475 | 0.7335 | −0.4085 |
| TP53INP1 | −0.5935 | 1.0425 | 0.8975 | 1.3255 | 0.4435 | −0.0435 | 0.1595 | −0.5985 | 0.9245 | 0.2435 | 0.3965 | −0.0005 | −0.0295 | 1.4275 | 1.4165 |
| TPD52L1 | 0.482 | −0.059 | −0.874 | −0.225 | 0.698 | 1.04 | 1.332 | 2.333 | 0.188 | −0.259 | 0.793 | 0.729 | 0.621 | −1.775 | −0.869 |
| TPRT | 0.8455 | −0.3535 | −0.7765 | −0.0325 | −0.3245 | 0.9505 | −0.0725 | 0.0905 | −0.2825 | 0.6545 | −0.6735 | 0.5455 | 1.8785 | 0.3175 | −0.1735 |
| TRA@ | 0.334 | −0.561 | 0.897 | 0.122 | −0.079 | −0.021 | −0.097 | −0.114 | 0.535 | 0.069 | −0.508 | 0.183 | −0.372 | −0.287 | −0.69 |
| TRAF4 | −0.531 | −0.715 | −0.878 | −0.632 | −0.026 | −0.22 | −0.424 | −0.56 | −0.218 | −0.44 | −1.038 | −0.175 | −0.372 | −0.145 | −0.45 |
| TRAPPC4 | −0.575 | −0.484 | −0.629 | −0.557 | −0.137 | −0.348 | 0.114 | −0.374 | 2.008 | 1.142 | 0.285 | 2.318 | 1.935 | 0.537 | −0.041 |
| TRH | −1.2635 | 0.4725 | 0.2055 | 0.8865 | −0.1385 | −0.1545 | 0.5335 | −0.8915 | −0.6355 | −0.7335 | 1.2305 | 1.6885 | 1.1285 | 0.3355 | 0.1505 |
| TRIM17 | −0.2305 | −0.2785 | 0.5915 | 0.8825 | 0.0065 | −0.1985 | 0.0085 | −0.8355 | −0.1785 | −0.0405 | −0.4305 | −0.6935 | 0.3885 | −0.9905 | −0.0755 |
| TRIM2 | −0.9485 | −0.8065 | −0.7295 | 0.1285 | 0.6285 | 2.1335 | −0.0555 | 0.6385 | −0.4185 | 0.4325 | −0.3495 | −0.1835 | −0.1125 | −0.0505 | −0.4205 |
| TRIM6 | −4.8 | 0.435 | −0.851 | −1.672 | −0.569 | −0.815 | −1.548 | −0.311 | 0.428 | 0.172 | 0.883 | 0.231 | −0.512 | −1.351 | −0.939 |
| TRIP3 | −0.1865 | −0.3295 | −0.3905 | −0.4415 | 0.0885 | −0.4865 | −0.8415 | −0.4455 | −0.8285 | −0.1725 | −0.0565 | −0.4905 | −0.4205 | 0.0865 | 0.3645 |
| TRPS1 | −1.0085 | 0.0095 | −0.6005 | 0.0375 | −0.1205 | −0.6645 | −0.0675 | −0.0805 | 0.5855 | −0.1985 | −0.6975 | −0.1875 | −0.3885 | −0.3065 | −0.9535 |
| TSG101 | −1.2195 | −0.1675 | −0.6865 | −1.4265 | −1.0685 | −0.2535 | 0.5035 | 0.9366 | 0.5835 | −1.0065 | −1.3515 | 0.1075 | −0.3805 | 0.1485 | 0.8995 |
| TTC10 | −0.0425 | 0.3945 | −0.1305 | −0.0715 | 0.5375 | −0.0035 | −0.2565 | −0.6225 | 0.0405 | 0.2105 | 1.0305 | 0.7855 | −0.5085 | 1.9295 | 0.9185 |
| TTC12 | 0.548 | 0.374 | 0.331 | 1.059 | 0.283 | −0.138 | −0.077 | 0.294 | 0.115 | 0.573 | 1.15 | 1.851 | 0.891 | 1.931 | 1.473 |
| TTK | 0.6395 | −0.2205 | 0.0035 | 0.5875 | 0.9545 | 0.3265 | 1.2345 | −0.4035 | 0.6555 | 0.5415 | 2.5775 | 4.0775 | 4.3015 | 4.0985 | 4.7735 |
| TTLL4 | 0.307 | 1.523 | 1.977 | 1.767 | 2.626 | −1.134 | 1.623 | −0.638 | 0.152 | 1.675 | 1.197 | 0.06 | 0.713 | 2.057 | 1.391 |
| TTYH1 | 0.4955 | −0.5915 | −0.2545 | −0.7825 | −0.6565 | −0.6305 | 0.7385 | 0.5395 | 0.5725 | 1.9065 | 1.3665 | 2.1835 | 0.5945 | 0.9945 | 0.6065 |
| TUBB3 | 0.0955 | 0.3305 | 0.1765 | 0.1525 | 0.3705 | −0.3595 | 0.3745 | −0.2435 | −1.0505 | −0.6245 | −0.0315 | −0.1505 | −0.0615 | 0.0385 | −0.1115 |
| TULP3 | 0.285 | 0.425 | 0.085 | 2.405 | 0.325 | 0.035 | 0.265 | 0.105 | −0.188 | −0.595 | 1.475 | 0.745 | 0.552 | 0.943 | 0.192 |
| TXN | 3.82 | −0.663 | −0.384 | −0.656 | −0.64 | −0.441 | 0.282 | 0.881 | 0.628 | −0.175 | 0.035 | 1.454 | −0.23 | 0.588 | 0.378 |
| TYMS | −0.6315 | 0.3385 | 1.1675 | 1.0805 | 1.1045 | 0.0445 | 0.6165 | −0.1345 | 0.1525 | 1.0405 | −0.0395 | 0.0895 | 0.1325 | 0.0145 | 0.9415 |
| U5-116KD | 0.4575 | −0.2825 | −0.3645 | 0.2425 | 0.1425 | −0.5885 | −0.5185 | −0.1575 | 0.4435 | −0.1535 | −0.7515 | −0.7835 | −0.6455 | −0.0935 | 2.2035 |
| UBE2E3 | 0.889 | −0.169 | −0.148 | −0.266 | −0.226 | 0.024 | −0.055 | 0.303 | 0.461 | 0.17 | 0.078 | 0.811 | −0.238 | −0.048 | 0.028 |
| UBL5 | −0.2235 | 0.1435 | 1.2645 | 1.0165 | 0.5755 | 0.5815 | 0.3275 | 1.6885 | −0.5745 | −0.0805 | 2.1885 | 0.1665 | 3.0275 | 3.1445 | 0.9765 |
| UBN1 | −0.391 | 0.785 | −0.429 | 1.201 | 0.034 | −0.187 | 0.201 | −0.942 | −0.355 | −0.527 | −0.154 | 0.417 | −0.276 | −0.125 | 1.37 |
| UCHL1 | −0.416 | −1.388 | −1.181 | −0.588 | −1.277 | −0.205 | −0.834 | 0.577 | −0.525 | −0.527 | −1.827 | −0.7 | −1.009 | −1.981 | −0.039 |
| UCHL3 | 1.623 | 0.868 | 0.288 | 0.409 | 0.429 | 0.499 | 0.823 | −0.712 | −0.425 | −0.217 | 0.45 | −0.162 | 0.109 | 0.415 | 0.935 |
| UGCG | 5.2245 | −0.1885 | 4.4025 | 2.2646 | 2.6275 | 3.2125 | 3.8925 | −1.1675 | 0.5735 | 0.0555 | −0.4645 | −1.6485 | 0.1545 | −0.5675 | −0.8105 |
| UGCGL1 | 0.0885 | −0.0105 | 3.3685 | 0.8255 | 0.2035 | 0.8365 | 3.1955 | 1.1735 | −0.0375 | 0.9275 | 1.3445 | 1.4005 | 0.6175 | 0.7905 | 0.3235 |
| UGT2B7 | 0.9965 | −0.2165 | 0.1805 | 0.6925 | 0.3755 | 0.1955 | 1.5225 | −0.0855 | −0.0845 | 0.6285 | 0.6025 | 0.5925 | −0.2485 | −0.4485 | 0.1205 |
| UNC13D | −0.701 | 0.134 | −0.075 | −0.283 | −0.18 | −0.477 | −0.776 | −0.104 | −0.762 | 0.021 | −0.492 | −0.828 | −0.389 | −0.518 | 0.018 |
| VAMP8 | 1.171 | −0.48 | 0.49 | 0.139 | 0.264 | 1.142 | 1.162 | 1.496 | −0.35 | 0.099 | −0.047 | 0.863 | 0.175 | −0.885 | −0.329 |
| VAPA | −0.8415 | −0.7465 | 0.0395 | 0.3535 | −0.3745 | −1.3225 | −1.9745 | 0.3185 | 0.6285 | −0.6035 | −2.1145 | −2.4415 | −3.0925 | −1.8925 | −0.5355 |
| VAV1 | 4.904 | −0.524 | −0.739 | −0.983 | −0.104 | 0.362 | −0.235 | −0.71 | 0.52 | 0.543 | 0.701 | 0.428 | 0.469 | 0.261 | 1.338 |
| VAV3 | 0.096 | 0.553 | 0.075 | 0.069 | 0.413 | −0.015 | −0.756 | −0.602 | −0.718 | −0.348 | 0.79 | −0.136 | −0.972 | −0.141 | 0.257 |
| VCX3 | −0.28 | 0.059 | 0.152 | −0.476 | 1.346 | −0.291 | 0.084 | −0.063 | −0.035 | −0.045 | 0.004 | −0.249 | 0.002 | −0.241 | −0.378 |
| VDAC2 | 0.2085 | 0.0385 | −0.0915 | 0.1285 | −0.1515 | 1.0385 | 3.0385 | −0.1315 | 0.2905 | 0.6485 | 2.5885 | 3.4885 | 1.1985 | 1.7075 | 0.8535 |
| VEST1 | −0.07 | 0.669 | −0.141 | −0.041 | −0.027 | −0.279 | −0.42 | 0.267 | 0.143 | 0.465 | 0.409 | 0.413 | 0.587 | 0.152 | −0.426 |
| VGLL1 | −0.2095 | −1.2215 | −0.9065 | −0.6795 | −0.4505 | −0.5735 | −0.6435 | 0.4025 | 0.0395 | −0.4425 | −0.0365 | 1.3305 | 0.9775 | −0.2505 | 0.3485 |
| VMD2L2 | 0.811 | 0.1685 | 1.591 | −0.094 | −0.003 | −0.422 | 0.843 | 0.875 | 0.571 | −0.255 | 0.574 | 0.459 | 0.121 | −0.231 | −0.63 |
| VSX1 | −0.294 | 0.711 | 0.858 | 0.12 | 1.097 | 0.297 | 0.188 | 0.312 | −0.145 | 0.186 | −0.364 | −0.056 | −0.31 | −0.222 | −0.583 |
| WARP | 0.268 | −0.578 | −0.382 | −0.387 | 0.028 | −0.002 | −0.439 | −0.105 | 0.356 | −0.638 | 0.243 | −0.092 | 0.18 | 0.216 | −0.078 |
| WDR26 | −1.5525 | −0.2895 | −0.5825 | −1.3945 | −0.3585 | −0.5485 | −0.2095 | 1.4595 | −0.3435 | 0.1615 | 0.3145 | −0.0615 | −0.7605 | −0.5845 | −0.5415 |
| WDR6 | 1.38 | −0.378 | −0.452 | 0.337 | −0.543 | −0.517 | 0.378 | −0.279 | −0.418 | −0.274 | 0.063 | 0.179 | 0.277 | −0.197 | 1.153 |
| WHSC1L1 | −0.369 | 0.661 | −0.209 | −0.309 | 0.021 | 0.071 | 1.341 | 0.491 | 0.178 | −0.209 | 1.871 | 4.401 | 2.45 | 1.001 | 0.124 |
| WNT3 | −0.1715 | −0.6045 | 0.1855 | 0.2505 | 0.0815 | 0.2425 | −0.1705 | −0.5525 | −0.5065 | 0.2385 | 0.0155 | 0.1965 | −0.4595 | −0.6955 | −0.2015 |
| WNT6 | −0.8465 | −0.5535 | 0.1125 | −0.9035 | −0.2555 | −0.1885 | 1.0325 | 0.8185 | 0.5725 | −0.4615 | −0.4065 | −0.5865 | 0.6275 | −2.1105 | −1.3525 |
| WSB2 | −0.1635 | −0.3485 | 0.2155 | 0.0015 | 0.3125 | 0.0315 | 0.2825 | 0.1025 | −0.0245 | −0.3245 | 0.3745 | 0.7735 | 0.5545 | −0.1765 | −0.0925 |
| WWP1 | −1.2395 | −0.3735 | −0.0105 | −2.3495 | −0.1185 | 0.7485 | −1.2275 | −0.5495 | 0.8055 | −1.1965 | −3.2185 | −3.2035 | −2.8515 | −3.8735 | −3.9365 |
| WWP2 | 0.15 | 0.981 | 0.137 | 0.52 | 0.242 | 1.283 | 0.559 | 0.418 | −0.755 | −0.741 | −0.035 | −0.53 | −0.226 | −0.358 | 0.197 |
| XBP1 | 0.365 | −0.004 | 0.749 | 1.381 | 0.994 | −0.496 | 1.122 | −0.216 | 0.965 | 0.808 | 1.428 | 1.489 | −0.428 | 0.118 | 0.241 |
| XYLT2 | 0.1825 | 0.3735 | 0.2195 | 0.4305 | 0.2035 | 0.1635 | 0.6905 | −0.2875 | 0.1775 | 0.3875 | 0.0045 | −0.0165 | −0.0355 | 0.2055 | 0.3645 |
| YBX2 | −0.815 | 0.223 | 0.724 | 0.636 | 0.617 | 0.234 | 0.006 | −0.206 | −0.32 | 0 | −0.696 | −0.885 | −0.925 | −1.23 | −0.297 |
| YIF1 | 0.8555 | −1.2515 | −0.7975 | −0.0895 | −0.9385 | 0.4025 | −0.1685 | −0.7585 | 0.8685 | −0.7895 | −1.6515 | −0.0825 | −0.7965 | −1.7365 | −1.2845 |
| ZA20D3 | −1.0235 | −0.9485 | −0.7745 | −0.7505 | −1.1165 | −0.5635 | −0.7825 | −0.3885 | −0.2885 | 0.0075 | −0.6095 | −0.0595 | 0.1055 | 0.4215 | 0.9265 |
| ZAP128 | −0.8795 | −0.7575 | −0.3116 | −0.7075 | −0.6065 | 0.0905 | −0.1495 | 0.1605 | −0.2725 | 0.6355 | −0.6335 | 0.5285 | −0.1005 | −0.3655 | −0.8875 |
| ZBED3 | 0.328 | −0.141 | 0.101 | −0.967 | −0.824 | 0.219 | 0.42 | 1.126 | 0.142 | 0.28 | 1.025 | 0.75 | 0.943 | 0.632 | 0.694 |
| ZBTB4 | 1.424 | 1.206 | 0.468 | −0.241 | −0.118 | 0.494 | 0.172 | −0.012 | −0.288 | −1.029 | −0.125 | −0.12 | −0.746 | 0.802 | −0.258 |
| ZCWCC1 | −0.0585 | 0.5445 | 0.2405 | 0.1645 | 0.4715 | 0.1885 | 0.0015 | −0.0095 | −0.6675 | −0.5225 | 0.5555 | −0.3875 | 0.0935 | 0.8565 | 1.4065 |
| ZCWCC2 | 1.276 | 0.062 | −0.384 | −0.928 | −1.533 | 0.285 | 0.388 | 0.991 | 0.702 | 1.713 | −1.256 | 0.287 | 0.809 | 0.388 | 0.534 |
| ZDHHC13 | −1.2285 | −0.0425 | 0.2485 | 0.0005 | −0.8155 | 0.1475 | 0.0925 | 0.7955 | −0.0005 | −0.3165 | −1.4015 | 0.0685 | −0.2755 | −2.2155 | −1.5205 |
| ZDHHC14 | −0.6025 | −0.5145 | −0.1865 | 0.2085 | 0.2115 | 0.4515 | −0.0435 | −0.4075 | −0.0745 | −0.6065 | −0.8105 | −0.8835 | −0.4575 | −0.6425 | −1.7625 |
| ZFHX4 | −0.0745 | −0.1355 | −0.4105 | −0.8995 | −1.0545 | 0.5245 | −0.4525 | 0.5185 | 0.6125 | 0.3745 | 0.3805 | 0.3835 | 0.4135 | 0.1905 | 0.7415 |
| ZFYVE21 | −0.321 | 0.203 | −0.306 | −1.433 | −0.263 | −0.749 | −1.595 | −0.599 | 0.014 | 0.476 | 0.38 | −1.122 | −1.621 | 0.068 | 0.508 |
| ZNF133 | −1.499 | −0.578 | −0.334 | −0.966 | 0.009 | −0.432 | −0.711 | −0.018 | −0.819 | 0.054 | −0.039 | 0.169 | 1.184 | −1.87 | 0.44 |
| ZNF165 | 0.8195 | −0.2855 | −0.1115 | −0.5455 | 0.1195 | −0.3205 | −0.3795 | −0.0225 | 0.0275 | −0.1745 | 0.6435 | 0.9035 | −0.2705 | 0.1275 | 0.6825 |
| ZNF24 | −0.717 | 0.513 | 0 | −0.088 | −0.214 | −0.037 | −0.253 | 0.07 | −0.08 | 0.147 | 2.241 | −0.173 | 0.112 | −0.425 | −0.34 |
| ZNF318 | 0.3915 | 0.0315 | 0.1435 | 0.1805 | 1.2235 | −0.3715 | −0.0845 | −0.0235 | 0.5425 | 0.2085 | 0.1005 | −0.3065 | 0.0015 | 0.2575 | −0.0675 |
| ZNF54 | −2.2305 | 0.8225 | −0.0395 | −0.3655 | 0.2455 | −0.5715 | −0.7595 | 0.1885 | −0.3535 | −0.0176 | 0.1485 | 0.3865 | 1.8835 | 0.3215 | 0.2055 |
| ZNF432 | 0.3875 | 0.3755 | 0.0205 | −0.0055 | 0.4485 | −0.3125 | −0.8925 | −0.3395 | 0.2565 | 0.1785 | 0.1115 | −0.9895 | −0.9295 | 0.2775 | −1.3635 |
| ZNF482 | −0.456 | 0.471 | 0.381 | −0.657 | −0.766 | 0.243 | −1.146 | 0.276 | 0.306 | −0.515 | −0.826 | −2.407 | −0.948 | −0.69 | −1.657 |
| ZNF505 | −0.625 | −0.799 | −0.183 | −1.004 | 1.374 | 0.264 | −1.071 | 0.533 | 1.435 | 0.056 | −0.586 | −0.354 | 1.062 | 0.123 | −0.547 |
| ZNF552 | −0.0995 | −1.0975 | −0.4255 | −1.0385 | −0.8215 | −0.3295 | −0.2415 | 0.8655 | 0.0025 | −0.2665 | −0.0285 | −1.1305 | −0.2325 | 0.1715 | −0.5895 |
| ZNF577 | 0.4545 | −0.0525 | 0.1325 | 0.0295 | −0.6895 | 0.0065 | −0.2545 | 0.1535 | 0.9975 | 0.5645 | 0.2545 | 0.2665 | −0.0215 | 0.6645 | 0.7716 |
| ZNF587 | 1.2225 | −0.5216 | −0.8175 | −0.4285 | −0.7925 | −0.4445 | −0.6675 | 0.2045 | −0.2425 | 0.1615 | 0.2275 | 0.0685 | −0.3895 | 0.0625 | 0.2755 |
| ZNF598 | −0.2765 | −0.2005 | −0.2195 | −0.5385 | −0.0115 | −0.2365 | −0.1465 | −0.0585 | −0.0885 | 0.0225 | −0.2065 | −0.4175 | −0.2215 | 0.4645 | −0.3065 |
| ZNF599 | −0.048 | −0.134 | −0.247 | 0.045 | 0.861 | −0.274 | −0.729 | −0.128 | 0.524 | 0.184 | 0.239 | −0.217 | −0.206 | 0.535 | 0.36 |
| ZNF606 | −0.74 | −0.679 | 0.122 | 0.131 | −0.34 | −0.129 | −0.298 | 0.3 | 0.046 | −0.796 | 0.118 | −0.131 | 0.572 | 0.345 | −0.189 |
| ZNF614 | −0.503 | −0.852 | 0.063 | −0.294 | −0.463 | 0.227 | −0.525 | 1.017 | 0.209 | −0.049 | −0.148 | 1.021 | 0.359 | 0.157 | 0.444 |
| ZNF639 | | | | | | | | | | | | | | | |
| ZNF7 | | | | | | | | | | | | | | | |

| | Sample | | | | | | | | | | | | | | |
|---|---|---|---|---|---|---|---|---|---|---|---|---|---|---|---|
| | 9830-0200 | 9830-0101 | 9830-0101 | 9830-0102 | 9819-0104 | BR02-705 [symbol] | BC00043T | BC00049T | BC00078T | BC00041 | UB-118 | BC00024T | BC00082T | BC00086 | BC00094T |
| | Class | | | | | | | | | | | | | | |
| Symbol | BASAL | BASAL | BASAL | BASAL | BASAL | BASAL | BASAL | BASAL | BASAL | BASAL | BASAL | BASAL | BASAL | BASAL | BASAL |
| | −1.8865 | −0.9655 | −1.4435 | −2.3565 | −2.4525 | −1.2815 | −1.9595 | −2.0575 | −0.6875 | −1.9335 | −1.5675 | −1.5275 | −1.3645 | −2.4355 | −1.0895 |
| | 0.797 | −0.4 | −0.422 | −1.038 | −0.542 | −0.353 | 0.065 | −0.237 | −0.174 | 0.733 | −0.15 | −1.019 | −0.392 | 0.05 | −0.417 |
| ABAT | −3.036 | −2.734 | 2.543 | −1.599 | −0.84 | −1.085 | −2.738 | −2.839 | −3.124 | −2.757 | −2.665 | −0.006 | −2.582 | −2.707 | −2.339 |
| ABC1 | −1.5855 | −1.0335 | 0.5875 | −1.0025 | −1.5375 | −0.7935 | 0.8795 | 0.2495 | 0.4235 | 0.8835 | −1.2175 | −0.2435 | −0.3305 | −0.1995 | −0.4875 |
| ABCC11 | −2.9565 | 0.9575 | −1.2385 | −0.4085 | −0.8745 | 0.1765 | −1.3455 | −0.2085 | −0.7505 | −0.4955 | 0.8475 | 0.6415 | −1.0645 | −1.9055 | 0.4235 |
| ABCC3 | −0.949 | 0.183 | −0.253 | 0.495 | 0.555 | 1.353 | 0.883 | 0.45 | −0.678 | −0.756 | −1.259 | 0.94 | −0.388 | 1.143 | 0.318 |
| ACAS2L | 0.9905 | −0.1455 | 0.9825 | 1.0945 | 0.1845 | 1.4875 | 1.7475 | 1.0815 | 1.1465 | −0.0385 | 0.3615 | 0.3245 | 0.8825 | 0.9565 | 0.5935 |
| ACAT1 | −2.976 | −1.541 | −2.865 | −1.585 | −1.673 | −0.91 | −1.392 | −1.890 | −0.647 | −1.81 | −3.656 | −0.135 | −1.285 | −1.029 | −1.508 |
| ACAT2 | −0.1735 | −1.1675 | −0.9475 | −0.9335 | −0.2885 | 0.3595 | −0.4885 | 0.0835 | −0.6835 | −0.5525 | −1.6555 | −0.3835 | −1.7355 | 0.6625 | −0.9035 |
| ACOX2 | −1.1685 | 0.3675 | 0.8885 | −0.4555 | −0.4095 | −0.3035 | 0.1095 | −0.0885 | −0.7015 | 1.3935 | 2.3955 | 1.3285 | 0.1255 | 0.4055 | −1.5305 |
| ACDX3 | 1.3545 | 0.8775 | −1.0285 | −0.4965 | −0.5415 | 0.5475 | 4.0195 | 3.5405 | 4.0395 | 3.8945 | −1.0415 | 0.1765 | 4.0985 | 0.4785 | −0.8085 |
| ACSL3 | 0.797 | −0.738 | −0.259 | −0.647 | −0.06 | 0.239 | 0.114 | 0.904 | −0.185 | −0.044 | 0.176 | −0.284 | 0.000 | −0.819 | −1.188 |
| ACTA1 | −0.0695 | 0.1545 | 0.0355 | 0.0185 | −0.0265 | −0.1085 | −0.0395 | −0.0115 | 0.4775 | 0.2815 | −0.0195 | −0.2605 | 0.2545 | −0.0185 | −0.1385 |
| ACTB | 0.5115 | 2.3245 | 1.7235 | 2.8995 | 0.8465 | 0.6785 | 0.1115 | 0.0235 | 0.7465 | −0.2535 | 1.4545 | 1.7975 | 0.7705 | 1.8425 | 0.9055 |
| ADAM33 | 1.278 | 2.628 | 2.927 | −0.886 | 1.951 | 2.24 | −1.291 | −0.831 | 0.662 | 0.352 | 0.898 | 2.768 | 1.941 | 1.563 | 0.29 |
| ADAMDE [symbol] | −0.8515 | 1.3295 | 1.2705 | −0.9085 | 0.1035 | 0.1015 | 0.3085 | −1.6475 | −0.1425 | −0.0925 | 0.2485 | 1.3155 | −0.3565 | 0.9665 | 0.9715 |
| ADPN | −1.4485 | −0.1215 | −1.3395 | 0.0095 | −1.0845 | 0.1845 | −1.2235 | −1.5775 | −0.4805 | −0.1895 | 0.0515 | −1.4085 | 0.7715 | −0.9025 | −1.0955 |
| ADPRHL1 | −4.7155 | −3.8885 | −4.5535 | −4.0485 | −4.1255 | −1.1975 | −5.4485 | −1.6045 | −4.4815 | −4.9925 | −5.1445 | −4.6345 | −4.7085 | −3.5505 | −4.2445 |
| ADRA2C | −0.1615 | −0.3905 | 0.7145 | 0.7515 | 2.3285 | −0.3685 | 1.0445 | −1.4435 | −0.9655 | −0.6125 | −1.5715 | 4.8145 | 1.9565 | 2.3615 | −1.3375 |
| AGR2 | −0.1545 | 0.4585 | 0.1145 | 0.1655 | −0.2925 | −0.0895 | −0.3605 | −0.3025 | 0.3185 | −0.2275 | −0.2885 | −0.4955 | 0.0275 | −0.1535 | 0.0105 |
| AGT | −0.101 | −0.559 | 0.250 | −0.637 | −0.24 | −0.274 | −0.506 | −0.019 | −0.551 | −0.39 | 0.002 | 0.192 | −0.193 | −0.133 | 0.131 |
| AGTR1 | 0.341 | 0.002 | 0.087 | 0.438 | −2.043 | −0.014 | −0.325 | 0.128 | −0.8 | −0.794 | −0.725 | −0.111 | 1.023 | −2.13 | −0.006 |
| AHCYL1 | 0.867 | −1.83 | −0.526 | 0.503 | −0.303 | 0.002 | 0.441 | 0.695 | 0.068 | −0.73 | −0.677 | −0.244 | 0.604 | 0.413 | −1.309 |
| AHNAK | 0.309 | −0.934 | 0.869 | 0.847 | −2.289 | 0.902 | 1.691 | −0.01 | 0.858 | 0.587 | 1.837 | 1.045 | 0.921 | −0.363 | −1.006 |
| AHR | 0.7835 | −0.0555 | −0.8475 | 0.4415 | 0.8635 | 0.5375 | 0.1215 | −0.2005 | −1.0395 | −0.1625 | 0.6815 | −0.3445 | 0.1755 | 0.8835 | −0.1145 |
| AIM1 | 0.4295 | 0.6385 | 0.2795 | 1.2785 | −0.6025 | −0.0525 | 0.1535 | −0.7525 | −0.1895 | −0.4955 | 0.0495 | 0.2255 | 0.6735 | 0.7435 | −0.3248 |
| AK2 | −0.2285 | 3.5125 | −2.1505 | 0.2185 | −0.1775 | 1.0515 | 1.3725 | −0.6135 | −0.1975 | −1.4495 | −2.4125 | −1.5435 | −0.5555 | −1.5535 | 0.6416 |
| AK3L1 | −1.533 | 0.604 | −0.058 | 0.442 | 0.574 | 0.134 | 0.859 | −0.198 | −0.412 | 0.528 | 0.996 | −0.223 | −0.065 | 0.071 | 0.804 |
| AK5 | −0.454 | −0.836 | 0.158 | 0.001 | −0.32 | −0.304 | −0.26 | −0.257 | 0.03 | −0.321 | 0.115 | −0.101 | −0.02 | −0.127 | −0.249 |
| AKAP1 | −0.1975 | 0.1415 | −0.0495 | 0.6605 | −0.0715 | 0.2975 | −1.1745 | −0.9095 | −0.5755 | −0.7505 | −0.5625 | −0.8785 | −0.0465 | −0.1245 | −0.8245 |
| AKR1A1 | −0.94 | 0.312 | 0.802 | 0.253 | −0.863 | 0.678 | 0.151 | −0.012 | −0.317 | −0.567 | 0.525 | 1.179 | −0.009 | 0.793 | 0.582 |
| AKR7A3 | 0.4725 | 0.4495 | −0.6005 | 0.7425 | 0.7665 | 0.3395 | 0.3765 | −0.1215 | −0.1885 | 0.2485 | 0.1085 | −0.2405 | 0.5885 | 1.0025 | −0.1005 |
| ALG5 | 1.4785 | −2.0735 | −0.7905 | −0.9665 | −1.7765 | 0.3645 | −2.1335 | −0.3915 | −0.6155 | −0.3045 | −1.0325 | 0.1275 | 0.4835 | 0.3495 | 0.4345 |
| ALG8 | 0.3245 | 0.7065 | 0.4645 | 0.1785 | 0.4805 | 0.2645 | 0.2875 | 0.5455 | 1.0415 | 0.2045 | 0.7255 | 0.3225 | 0.2875 | 0.3455 | 0.7205 |
| AMFR | 0.478 | −0.003 | 0.25 | −0.164 | −0.300 | 0.082 | 0.204 | −0.083 | 0.131 | 0.228 | −0.05 | −0.034 | −0.283 | 0.849 | 0.355 |
| AMPD2 | −1.03 | −1.307 | 1.566 | 0.573 | −0.51 | −0.693 | −0.34 | 0.377 | −1.474 | −1.154 | 1.709 | 0.121 | −0.227 | −0.766 | 1.365 |
| ANAPC11 | 0.247 | 0.759 | 1.159 | −0.07 | 1.692 | 1.009 | 0.457 | 1.33 | 0.827 | 0.838 | 0.333 | 0.227 | 0.81 | −0.942 | 0.785 |
| ANKRD22 | 0.678 | 0.793 | 1.387 | 0.85 | 1.547 | 1.138 | 2.281 | 1.599 | 0.795 | 1.174 | 1.227 | 1.828 | 1.408 | 0.974 | −0.321 |
| ANKRD27 | −0.432 | −0.001 | −0.011 | −0.58 | −0.753 | 0.295 | −0.259 | −0.259 | −0.591 | 0.26 | 0.879 | 0.579 | −0.038 | −0.586 | 0.002 |
| ANLN | 1.648 | 0.999 | 1.277 | 1.175 | −2.267 | 0.228 | 2.649 | 2.152 | 1.671 | 1.983 | −0.375 | 3.028 | 2.329 | 0.812 | 1.173 |
| ANP32A | −1.306 | −1.083 | −2.222 | −1.231 | −0.703 | −0.329 | 0.218 | −0.426 | −0.783 | −0.942 | −1.405 | −0.171 | −0.595 | −0.846 | −1.071 |
| ANXA3 | 1.5885 | 1.5785 | 4.5425 | 4.1845 | 3.2885 | 0.2615 | 1.9705 | 2.0395 | 1.8825 | −0.0925 | 0.1895 | 4.5735 | 3.1225 | −0.0605 | −0.9485 |
| APBB2 | 2.2825 | 1.7775 | 3.4595 | 3.7975 | −0.3905 | 3.0695 | 3.6885 | −0.8705 | 1.6805 | 1.9915 | 2.7405 | 1.4825 | 0.1025 | 0.1955 | −1.0725 |
| APOBEC3 | −5.457 | −4.219 | −5.412 | −2.521 | −0.309 | −1.51 | −4.141 | −4.363 | −3.108 | −3.45 | −2.137 | −4.062 | −3.378 | −4.894 | −3.78 |
| AQP5 | −0.982 | 0.142 | −1.463 | −0.72 | 1.238 | 0.943 | −1.384 | −2.149 | −1.239 | 3.144 | 1.738 | −2.228 | −1.918 | −1.193 | −1.06 |
| AR | 0.1525 | 0.3545 | 0.3895 | −0.2776 | 0.0955 | 0.2865 | 0.3555 | 0.4415 | 0.1565 | −0.3155 | 0.1725 | 1.4315 | 0.2575 | 1.1445 | 0.4125 |
| AREG | −1.21 | −0.971 | −0.285 | 0.467 | −0.583 | −0.384 | −0.876 | 0.541 | 0.325 | −0.881 | −0.231 | 0.975 | 0.559 | −0.664 | −0.209 |
| ARF1 | 0.2335 | −0.7715 | −0.2305 | −0.0635 | −0.1225 | 0.6965 | −0.2675 | 0.6215 | 0.4035 | 0.0355 | 0.3955 | −0.0935 | 0.0125 | 1.0175 | 0.3385 |
| ARHGDI [symbol] | 1.189 | 8.897 | 0.156 | 0.257 | 0.543 | 0.068 | 1.512 | 1.328 | 1.231 | 0.893 | 2.355 | 2.119 | 0.89 | 1.474 | 1.337 |
| ARL6IP | −0.0435 | −0.4645 | −1.1695 | −0.3545 | 0.2855 | 0.3025 | 0.3325 | −0.0875 | 0.8245 | 0.2905 | 0.4295 | −0.0955 | −0.1125 | −0.3205 | 0.4355 |
| ARL6IP2 | 0.005 | −2.515 | 0.221 | −3.235 | −2.897 | −1.368 | −0.155 | −0.533 | −1.28 | −0.479 | 0.907 | 0.194 | −0.147 | −1.682 | 0.396 |
| ARMCX2 | −0.042 | −2.505 | −0.339 | −3.539 | −3.033 | −1.594 | −0.043 | −0.589 | −0.941 | −0.277 | 0.74 | 0.275 | −0.188 | −2.257 | 0.387 |
| ARNT2 | 1.594 | 2.412 | 0.369 | 0.641 | 1.007 | 2.118 | 2.115 | 1.002 | 1.958 | 3.485 | 2.385 | 2.523 | 1.511 | 4.189 | 3.873 |
| ARNT2 | 0.7665 | 0.8225 | 0.1675 | 0.4535 | −0.9655 | 0.3055 | 0.0675 | −0.0355 | 0.0335 | −0.8435 | 0.2505 | 0.1425 | 0.8575 | 0.5185 | 0.2985 |
| ARP3BET [symbol] | −0.2235 | 0.0525 | −0.1175 | 1.1705 | 0.5585 | 0.1365 | −0.4725 | −0.2525 | 0.1325 | −0.2305 | −0.1125 | 1.4225 | 0.4715 | 0.5335 | 0.6135 |
| ARPC1B | −0.392 | 1.067 | 2.334 | 0.027 | 0.238 | 0.688 | 1.197 | −0.125 | 0.215 | −0.821 | −1.025 | 1.437 | 0.76 | 1.768 | 1.481 |
| ARPC5L | 1.3765 | −0.0875 | 0.8055 | 1.1435 | 1.0155 | 0.9295 | 1.9625 | 0.2925 | −8.4585 | 0.4005 | 2.0755 | 1.9745 | 1.2275 | 0.8965 | 0.6555 |
| AS89 | 0.057 | −1.19 | 0.712 | −0.659 | −0.604 | −0.224 | −0.046 | 0.559 | −0.395 | 0.037 | 0.229 | −0.104 | −0.5 | −0.023 | 0.217 |
| ASF1A | 0.9305 | 0.8075 | 1.0745 | 1.6275 | 1.3755 | 0.5825 | 1.8125 | 1.1405 | 0.3725 | 0.2005 | 0.4945 | 1.3455 | 0.8025 | 0.8825 | 0.8125 |
| ASH2L | 0.949 | −0.21 | 1.529 | 1.544 | 1.891 | 1.215 | 1.804 | 2.111 | 1.199 | 1.917 | 2.003 | 1.538 | 1.015 | 1.491 | 1.804 |
| ASK | 0.835 | 0.585 | 1.088 | 1.407 | 0.395 | 1.146 | −0.161 | −0.175 | −0.203 | 0.221 | −0.763 | −0.016 | −0.489 | 0.913 | −1.033 |
| ASPM | 1.7965 | 0.4425 | 1.8985 | 1.2995 | 2.3785 | 2.0945 | 1.7455 | 1.0795 | −0.4555 | 0.6775 | 1.2165 | 1.4485 | 0.1545 | 0.6445 | 0.3545 |
| ASS | 0.3085 | 0.5185 | 0.7725 | −0.1385 | 0.0515 | 0.6275 | −0.1305 | 0.5855 | 0.5565 | −0.4895 | 0.0215 | −1.5715 | −0.3455 | −0.6205 | 2.3585 |
| ATAD2 | −1.2075 | 0.4545 | −1.0905 | 0.1655 | 1.0055 | −0.5715 | −1.1235 | −0.2765 | −0.4985 | 0.0605 | 0.3155 | −0.3385 | 0.5495 | 1.3605 | 0.4485 |
| ATF5 | 0.035 | −0.422 | −0.053 | −0.592 | −0.511 | 0.173 | −0.561 | −0.61 | −1.127 | −0.812 | 0.078 | −1.428 | −0.937 | −0.346 | −0.708 |
| ATF7IP2 | −0.413 | 0.521 | −0.018 | 0.323 | −0.225 | 0.25 | 0.621 | −0.947 | −0.458 | −0.243 | 0.81 | −0.325 | 0.041 | 0.509 | 0.565 |
| ATP2B1 | 0.142 | 0.096 | −0.035 | 0.509 | −0.31 | 0.594 | 0.431 | −0.74 | 0.426 | −0.116 | 0.177 | −0.057 | 0.029 | 1.935 | 0.976 |
| ATP5G1 | 0.3095 | −0.3595 | −1.0415 | −0.9385 | −0.4695 | 0.0025 | −0.7675 | −0.6015 | −0.7775 | −0.4375 | −0.2185 | 0.4315 | −0.9335 | −0.1125 | −0.3845 |
| ATP5O | 1.832 | 4.478 | 3.818 | 3.556 | 0.754 | 2.069 | 0.361 | 0.814 | 0.009 | −0.544 | 0.558 | 1.095 | −0.517 | 3.329 | −0.773 |
| ATP6AP1 | −1.078 | −0.617 | 0.671 | −0.533 | −0.151 | 0.281 | 1.022 | 1.121 | −0.38 | −1.199 | −0.119 | −0.96 | 0.051 | 0.697 | −0.322 |
| ATP6V0A4 | −0.931 | 0.136 | −0.482 | 0.401 | −0.026 | −0.808 | −0.865 | −1.061 | −0.365 | −0.25 | −0.928 | 1.272 | 0.719 | 0.518 | −0.085 |
| ATP6V1B [symbol] | 0.0035 | −0.4535 | −0.7945 | 0.5185 | −0.9525 | 0.4355 | −0.2385 | −0.8395 | 0.8035 | −0.7255 | −0.2345 | −0.6285 | 0.3035 | 0.0915 | 0.3645 |
| ATP6V1G [symbol] | −0.381 | −0.416 | 0.352 | 0.199 | −0.074 | 0.154 | 0.018 | 0.08 | −0.446 | −0.085 | −0.489 | 0.243 | 0.297 | −0.192 | −0.421 |
| ATPIF1 | 1.0115 | 1.6595 | 3.2365 | 2.4825 | 2.0835 | 1.0955 | 3.4445 | 2.0835 | 2.2605 | 2.2835 | 2.8385 | 3.6065 | 2.9145 | 1.9225 | 4.0565 |
| ATXN10 | 1.965 | −0.319 | 0.508 | −0.16 | 0.003 | −0.253 | 0.516 | 0.809 | 0.383 | 0.269 | 1.108 | 1.21 | 1.023 | 2.168 | 1.295 |
| B3GNT5 | 2.5575 | −1.0185 | −0.8135 | −0.5075 | −0.1865 | −0.4945 | −0.9545 | −1.0845 | −0.7255 | −0.4915 | −0.4455 | −0.6985 | −0.2265 | 2.3105 | −0.6305 |
| BACE2 | 0.4815 | −0.7295 | 1.0216 | −0.1455 | −0.2895 | 0.0505 | 0.1645 | 0.2935 | −0.2095 | 0.2795 | −0.2905 | 0.1585 | −0.0015 | −0.2225 | 0.1135 |
| BAG1 | −0.5415 | −1.9035 | −1.0895 | −1.7585 | −1.7165 | −1.6265 | −0.5825 | −0.5275 | −0.6345 | −1.1125 | −0.6795 | −1.4265 | −0.4975 | −1.2395 | −1.0395 |
| BAG4 | 2.1665 | 0.8745 | 0.9865 | −0.0525 | −0.1205 | 0.0475 | 1.6485 | 0.4335 | −0.7665 | 0.8635 | 2.0295 | 2.8055 | −0.4155 | 0.1145 | 3.6445 |
| BAI2 | −1.524 | −1.04 | −1.15 | −1.24 | −1.977 | −1.653 | −0.963 | −1.231 | −1.658 | −1.006 | −2.445 | −0.999 | −0.968 | −1.041 | −1.332 |
| BAMBI | 0.525 | 1.819 | 1.962 | 1.553 | 2.013 | 0.108 | −0.226 | −0.268 | 1.98 | 0.358 | 3.014 | −0.256 | 1.386 | 2.282 | 1.521 |
| BCAS1 | −5.687 | −4.326 | −5.653 | −5.612 | −4.163 | −2.549 | −4.857 | −5.218 | −4.352 | −4.489 | −4.784 | −5.055 | −3.891 | −5.459 | −4.558 |
| BCL2L14 | 2.4705 | 0.7225 | −0.1115 | −1.1805 | −0.5725 | −0.2516 | −0.8315 | −0.0255 | −0.3195 | −0.7135 | −0.0075 | 0.4595 | −0.0135 | −0.8805 | −0.7275 |
| BCMP11 | 1.319 | −0.150 | −1.802 | −2.425 | −1.348 | −0.121 | 0.172 | −0.123 | 0.735 | −0.342 | 0.713 | 1.683 | 0.984 | −0.778 | −0.593 |
| BEX1 | −0.1555 | −0.8765 | 0.8135 | −1.6835 | −0.0235 | −0.7195 | −1.6265 | −0.0365 | −1.3755 | −2.9735 | −1.6605 | −1.3725 | 0.3885 | 2.3365 | −1.9575 |
| BEX2 | 0.2845 | 1.1555 | −0.2265 | 0.7285 | −0.1085 | 0.2895 | 0.7615 | −0.7165 | −0.1275 | 0.3345 | 1.2305 | 1.4685 | −0.8505 | −0.4825 | −0.3955 |
| BF | −0.885 | 0.583 | 0.039 | 0.308 | −0.348 | −0.165 | −0.73 | −0.67 | 0.605 | −0.213 | −0.48 | −0.349 | 0.039 | −0.343 | −0.81 |
| BFSP1 | −0.659 | −0.435 | −0.266 | 0.177 | −0.352 | 0.185 | 1.537 | 1.637 | 0.304 | 1.432 | −0.589 | 1.874 | 0.718 | 0.183 | 0.99 |
| BIK | 0.98 | 0.206 | 0.921 | 0.741 | 0.913 | 1.506 | 0.92 | 1.729 | −0.12 | 0.937 | 1.83 | 0.896 | 0.327 | 1.312 | 1.3 |
| BIRC2 | −0.5495 | −0.0365 | −0.0065 | 0.0275 | 0.3585 | −0.4365 | 0.8325 | −0.2805 | 0.4245 | 0.5055 | 0.3725 | −0.3505 | 0.9085 | 1.8945 | 0.8555 |
| BIRC5 | 0.1395 | 0.0755 | 1.8705 | 1.1225 | 0.3085 | 1.1445 | 0.5835 | 0.2775 | −0.4135 | 1.2115 | 1.4865 | 0.7045 | −0.5255 | 0.0215 | 1.6635 |
| Bit1 | −0.7435 | −2.3675 | −0.7735 | −1.7655 | −2.0255 | −1.3055 | −0.1145 | −0.5085 | −1.4285 | −1.9055 | −1.2825 | −1.8185 | −0.3655 | −0.7885 | −0.6585 |
| BIVM | 0.934 | 0.288 | 1.606 | 0.999 | 0.951 | 1.149 | 1.515 | 0.092 | −0.111 | 0.595 | 1.115 | 1.348 | 0.941 | −0.282 | 1.435 |
| BLVRA | 2.121 | −1.191 | 1.381 | −1.462 | −1.156 | 1.434 | 1.438 | −1.235 | −0.238 | −0.961 | 2.043 | −0.648 | 2.735 | −1.476 | −0.597 |
| BM039 | −0.005 | 1.419 | 0.858 | 0.209 | 0.968 | 1.683 | 1.408 | 0.377 | −0.216 | 0.004 | 0.722 | −0.229 | 0.202 | −0.725 | −0.058 |
| BMP7 | −1.251 | −2.741 | −0.406 | 0.513 | 0.269 | 0.366 | −1.719 | −0.075 | 1.366 | 0.381 | −0.314 | −0.891 | 0.546 | 0.906 | 0.806 |
| BOC | 0.3855 | −1.5645 | 1.0595 | −0.1145 | −0.1435 | −0.1125 | 0.2315 | 0.5055 | −0.3535 | −0.2365 | 0.2265 | 0.3115 | −0.2125 | 0.3945 | 0.9975 |
| BRCA1 | −0.0865 | 1.1235 | 0.6815 | 1.0055 | 0.7965 | −0.0365 | 1.3585 | 0.8745 | 0.8835 | 0.8695 | −0.0595 | 1.6385 | 1.0315 | 1.3075 | 0.8735 |
| BRF2 | 0.998 | −0.894 | −0.168 | 0.155 | −0.261 | 0.573 | −1.175 | −0.868 | −1.01 | −0.387 | 0.565 | −0.294 | −0.155 | 0.805 | 0.095 |
| BRI3 | −1.223 | 1.965 | 0.903 | 0.702 | 0.822 | 0.457 | 0.025 | −0.975 | −0.808 | 1.148 | 0.707 | 1.765 | −1.409 | −0.585 | −0.397 |
| BRI3BP | −0.809 | −0.649 | −1.324 | −0.414 | −1.123 | −0.48 | −0.51 | −0.696 | −0.676 | 0.038 | −1.153 | −0.884 | −0.162 | −0.550 | −0.242 |
| BSPRY | 2.1405 | −0.2005 | 1.6455 | −0.1435 | 1.4565 | 1.1715 | 0.8195 | 0.8845 | 1.1095 | 1.6035 | −0.1805 | 1.2405 | 1.5425 | 2.8515 | 2.5805 |
| BTF3 | −0.1405 | −0.4875 | 0.2845 | 0.6335 | −0.6515 | −0.2745 | 0.1165 | 0.6485 | 0.8815 | −0.1885 | 1.2485 | 1.5045 | −0.0175 | 0.2025 | 1.9075 |
| BTG3 | 1.579 | −0.095 | 1.506 | 1.38 | 1.448 | 1.294 | 0.751 | 1.153 | 0.903 | 1.048 | 1.466 | 0.96 | 0.873 | 0.726 | 0.263 |
| BγN2A2 | 0.722 | 0.936 | 0.543 | 1.323 | 0.191 | 0.275 | −0.52 | −0.032 | 0.058 | −0.21 | −0.855 | −0.223 | −0.893 | −0.255 | −0.084 |
| BUB1 | 0.6505 | 0.3285 | 0.7565 | 1.1485 | 0.3645 | 0.5595 | 2.2505 | 0.9265 | 0.9465 | 0.8135 | 1.2585 | 2.8505 | 1.9675 | 1.1405 | −0.3105 |
| BUB3 | −0.03 | 0.546 | 0.694 | 1.325 | 0.517 | 0.252 | −0.033 | 0.771 | −0.235 | 0.435 | −0.364 | 0.924 | 0.12 | 0.322 | 0.42 |
| BXDC1 | 0.347 | −0.571 | −1.017 | 0.17 | −0.906 | −0.426 | 0.068 | −0.419 | −0.181 | −0.161 | −0.484 | −0.279 | 1.751 | −0.152 | −0.381 |
| BZRP | −0.876 | −0.154 | −2.815 | −0.644 | −3.887 | −0.662 | −2.827 | −3.826 | −1.2 | −1.64 | −3.144 | −4.384 | −2.624 | −0.134 | −1.345 |
| BZW1 | 0.2995 | −0.6475 | 0.0085 | −0.6685 | 1.7995 | 0.9435 | −0.3025 | 0.8555 | 0.4545 | 1.2365 | 1.6305 | 1.0265 | 0.6335 | 0.7325 | 1.7755 |
| C10orf116 | 0.2035 | −1.3055 | −0.5965 | −0.9785 | −0.8365 | 0.0145 | −0.1575 | −0.2495 | 0.3575 | 0.0505 | 0.5835 | −0.9225 | −0.3185 | −0.6555 | −0.3275 |
| C10orf38 | 0.206 | −0.349 | −1.197 | −0.742 | −0.494 | 0.012 | −0.185 | 0.626 | −0.837 | 0.019 | 0.095 | 0.368 | 1.185 | 0.095 | −0.096 |
| C10orf4 | 0.2405 | 0.5375 | 1.2505 | 0.8825 | 2.0235 | 1.4035 | −0.1195 | 0.9765 | 0.8175 | 0.3125 | 0.7105 | 1.1555 | 1.4325 | 0.9345 | 1.3535 |
| C10orf45 | −0.9395 | −1.2235 | −1.4505 | −0.9355 | −0.2355 | 0.0165 | −0.6225 | −0.5135 | −0.3935 | −0.3145 | −1.2585 | −0.3955 | −0.7575 | −0.4985 | −0.3375 |
| C10orf7 | 0.8845 | 0.2655 | −0.3505 | 0.2245 | 0.2615 | −0.0235 | 0.5235 | 0.7045 | 0.4735 | 0.4715 | −0.1515 | −0.3575 | 0.5405 | 0.1105 | 0.1695 |
| C10orf82 | −0.758 | 1.424 | 1.513 | 0.338 | 0.155 | 0.312 | −0.178 | 0.934 | 0.523 | 0.521 | 1.656 | −0.18 | 0.622 | −0.278 | 1.132 |
| C11orf24 | −1.1265 | −1.5775 | −1.7615 | −0.5825 | −0.2715 | −0.1245 | −0.4175 | −0.8065 | 0.3255 | −0.8945 | 0.8785 | 0.3695 | 0.4915 | −1.6255 | −0.9415 |
| C12orf14 | 0.398 | 1.014 | 1.158 | 0.536 | 1.443 | 0.176 | 0.305 | 0.395 | 0.446 | 0.388 | 0.875 | 2.308 | 0.234 | 1.427 | 0.953 |
| C13orf21 | −0.035 | −1.613 | 0.131 | −0.299 | 0.491 | 0.474 | −0.278 | 0.008 | −0.063 | 0.148 | −0.73 | −0.537 | −0.895 | −0.457 | −0.202 |
| C13orf23 | 0.278 | 1.081 | 0.982 | 1.182 | 0.804 | 0.7 | 0.8 | 0.651 | 0.933 | 0.196 | 0.428 | 0.596 | 1.222 | 1.256 | 0.699 |
| C14orf94 | −1.34 | −1.58 | −1.47 | −0.99 | −1.31 | −0.525 | −1.01 | −0.84 | −0.01 | −1.08 | −1.96 | −2.07 | −0.328 | −1.309 | −0.181 |
| C15orf23 | −0.3555 | −0.4405 | −0.0985 | −0.6765 | −0.7905 | −0.7565 | −0.7875 | −0.8915 | −1.0305 | −0.3385 | −0.4755 | 0.4585 | −0.3065 | −0.4735 | −0.8145 |
| C16orf45 | −0.764 | 0.571 | −1.191 | −1.068 | −0.915 | −0.842 | 0.179 | 1.271 | 0.08 | 0.53 | 1.743 | −0.198 | −0.399 | −1.587 | 0.777 |
| C17orf37 | 0.313 | −0.512 | 0.352 | 0.437 | 0.193 | 0.246 | −1.033 | −0.455 | −0.086 | −0.614 | 0.163 | −0.582 | 0.859 | 1.364 | 0.324 |
| C18orf1 | −1.414 | −1.167 | −1.561 | −1.084 | 0.031 | −1.316 | −0.996 | −1.55 | −1.71 | −1.208 | −2.156 | −1.281 | −1.535 | −1.37 | −1.928 |
| C19orf10 | −0.8685 | −0.7065 | −0.8835 | −0.5705 | −0.5705 | −0.9765 | −0.3095 | −0.0085 | 0.0745 | −0.5785 | −1.1285 | −0.3615 | −0.1775 | −0.3515 | −0.2315 |
| C19orf32 | −3.3785 | −1.6545 | −0.0415 | 1.3485 | −2.5435 | 0.0495 | 0.0375 | −0.9495 | 0.3185 | −1.5885 | 0.1235 | 1.6535 | −1.1745 | −1.5505 | 0.9555 |
| C19orf33 | −0.551 | −2.178 | −1.956 | −1.234 | 1.32 | 0.386 | −1.552 | −1.926 | −1.754 | 0.577 | −2.597 | 0.507 | −0.33 | −1.48 | −3.771 |
| C1orf24 | −0.613 | −2.035 | −2.388 | −1.16 | −1.231 | 0.431 | −0.062 | −0.239 | 0.54 | −0.852 | −0.644 | −1.835 | −1.422 | −0.902 | −0.91 |
| C1orf34 | 0.8375 | −0.0925 | −0.5455 | −1.2435 | 0.0126 | −0.5445 | −0.9925 | −0.2385 | −1.3115 | 0.0235 | −1.2565 | 0.1576 | −0.5355 | −1.0025 | −0.3955 |
| C20orf103 | 0.6765 | −1.2435 | −0.7235 | 0.2815 | −0.2095 | −0.0525 | 0.0485 | −1.2865 | −0.9595 | −0.6375 | −0.9565 | 0.6115 | −0.2555 | −1.9175 | −1.1385 |
| C20orf149 | −0.31 | −1.21 | −1.795 | −0.604 | 0.45 | 0.459 | −0.459 | 1.119 | 0.045 | −0.769 | −0.905 | 1.051 | −0.119 | 0.168 | 0.017 |
| C20orf23 | −0.004 | 0.278 | 0.862 | 0.116 | −0.329 | −0.236 | −0.522 | 0.038 | −0.922 | −0.476 | 0.258 | 0.316 | 0.057 | −1.084 | −0.044 |
| C20orf3 | 0.185 | 0.349 | 0.346 | −0.946 | 0.565 | 0.118 | −0.45 | −0.202 | 0.168 | −0.249 | 0.024 | 0.407 | 0.092 | −0.231 | 0.095 |
| C20orf35 | 0.647 | 0.692 | 0.429 | 0.843 | −0.332 | 0.921 | 0.811 | 0.331 | 0.578 | 0.215 | 0.117 | 0.925 | 0.898 | 0.542 | 0.188 |
| C20orf43 | 0.8435 | 0.0785 | 0.3775 | 0.2655 | 0.7965 | 0.2285 | 0.0695 | 0.0355 | −0.0265 | 0.0145 | −0.3735 | −0.7685 | 0.0035 | 0.9115 | 0.9825 |
| C20orf8 | −0.04 | 0.078 | 1.697 | 0.082 | 1.208 | 0.814 | 0.016 | 0.225 | −0.212 | −0.007 | 1.119 | 0.659 | 0.101 | −0.942 | 0.162 |
| C21orf45 | −0.731 | −1.035 | 0.475 | −1.048 | −0.682 | 0.737 | −0.305 | 0.845 | 0.44 | 0.388 | 0.355 | 0.45 | 0.483 | 0.491 | 0.573 |
| C22orf18 | −0.884 | −0.041 | −0.647 | −0.632 | 0.176 | 0.02 | 0.059 | 0.282 | 0.281 | −0.139 | −0.598 | 0.384 | 0.086 | −0.05 | 0.158 |
| C2F | −0.841 | 0.263 | −0.04 | −0.039 | −1.356 | −0.243 | −1.483 | −1.528 | −0.667 | −0.732 | −1.326 | −1.227 | −0.537 | 0.468 | −1.498 |
| C2orf17 | −0.008 | −0.321 | −1.073 | 0.303 | 0.843 | 1.295 | 0.942 | 0.117 | −0.024 | 0.051 | −0.884 | −1.037 | 0.248 | 0.208 | 0.748 |
| C4A | 1.044 | 0.424 | 1.258 | 1.778 | 0.822 | 0.198 | 0.192 | 0.243 | 0.805 | 0.192 | −0.095 | 0.898 | −0.2 | 0.782 | 0.992 |
| C5orf13 | −0.038 | −0.455 | −0.563 | 0.245 | 0.456 | 0.224 | −0.022 | 0.158 | 0.193 | 0.521 | 0.927 | 1.257 | 0.395 | 0.666 | 1.202 |
| C6orf108 | 0.808 | 0.236 | 0.288 | 2.08 | −1.632 | −0.039 | 0.554 | 0.315 | 0.79 | −1.073 | 0.994 | −0.359 | 1.586 | 1.898 | 1.187 |
| C6orf166 | −0.392 | −1.293 | −0.51 | −0.001 | −1.114 | −0.6 | −0.542 | −0.754 | −1.201 | −1.995 | −1.093 | −0.862 | −0.41 | −0.033 | −0.356 |
| C6orf192 | 0.4205 | −0.0165 | −0.2435 | 0.6905 | 0.0375 | 0.2475 | 0.1765 | 0.5865 | −0.4585 | −0.2925 | 0.2595 | 0.3795 | 0.1075 | 0.3835 | −0.0945 |
| C6orf211 | −1.938 | 0.006 | −2.945 | −1.673 | −2.826 | −0.852 | −0.638 | −0.814 | −1.3 | −0.807 | −1.912 | −0.169 | −0.741 | −1.923 | −0.354 |
| C6orf49 | −0.7055 | 0.3115 | 0.8555 | 0.0325 | 0.8315 | 1.3385 | −0.1905 | −0.8795 | 0.0605 | −0.4005 | 0.0385 | 0.7555 | −0.7085 | 0.0265 | −0.3865 |
| C6orf85 | −0.1345 | 0.8335 | 1.4265 | 0.5755 | 0.1705 | −0.8825 | −1.1675 | −0.6425 | −0.1085 | 0.3365 | 0.0075 | −0.6115 | −0.4655 | −0.9095 | −0.6385 |
| C7orf24 | 1.956 | −0.612 | −0.330 | −0.775 | −1.448 | −0.336 | −0.356 | −0.907 | −1.201 | −1.434 | −1.006 | −0.504 | −0.458 | −0.906 | −0.048 |
| C9orf10 | 0.976 | 2.439 | 0.333 | 1.745 | 0.574 | 1.693 | 1.571 | 1.741 | 1.851 | 1.903 | 1.658 | 2.71 | 1.505 | 2.14 | −0.652 |
| C9orf121 | −0.436 | 0.387 | −1.172 | −0.52 | −1.829 | −0.338 | −1.101 | −0.683 | −0.593 | −0.093 | −0.185 | −0.008 | −0.45 | −0.597 | −0.332 |
| C9orf58 | 0.4785 | 1.3425 | 0.3495 | 0.8065 | 0.8195 | 0.1315 | −0.1135 | −0.6505 | 0.1625 | 0.1705 | 0.7075 | 1.0455 | 0.0575 | −0.0175 | 1.0495 |
| C9orf88 | −2.388 | −2.648 | −3.467 | −2.489 | −2.042 | −2.032 | −1.579 | −1.172 | −2.72 | −2.455 | −3.577 | −2.083 | −2.524 | −1.13 | −2.828 |
| C9orf9 | 0.18 | −0.853 | −1.845 | −1.732 | −2.028 | −0.999 | 0.835 | 0.823 | 0.105 | −0.827 | 0.998 | −1.339 | −1.101 | −0.05 | −0.895 |
| CA12 | 0.283 | −1.123 | −3.017 | −2.71 | −2.212 | −0.713 | 0.888 | 0.811 | −0.144 | −0.892 | 0.81 | −1.393 | −0.89 | 0.118 | −1.542 |
| CA8 | 0.203 | 0.834 | 0.308 | −0.067 | −0.115 | −0.146 | −0.538 | −0.327 | 0.13 | −0.169 | −0.556 | −0.484 | 0.006 | −0.323 | 0.255 |
| CA8 | 7.0355 | −0.0795 | 0.0465 | 0.5845 | 0.1715 | 0.5375 | −0.1205 | −0.1755 | −0.2245 | −0.0725 | 0.2395 | 0.0015 | 0.0985 | 0.2255 | 0.0875 |
| CACNA1G | 0.907 | 1.429 | 1.911 | 0.927 | 0.838 | 0.505 | 0.261 | −0.112 | 0.81 | 0.315 | 0.582 | 0.694 | 0.918 | 0.028 | 0.735 |
| CALCA | 2.380 | 3.34 | 4.85 | 3.426 | 2.943 | 3.381 | 2.609 | −0.496 | 2.94 | 1.369 | 3.828 | 1.174 | 2.898 | −0.628 | −0.318 |
| CALML4 | 0.3375 | −1.5125 | −2.6405 | −1.4835 | −1.8885 | −0.9685 | −1.5185 | −0.7155 | −1.7975 | −1.5235 | −1.3995 | −1.1565 | 0.0395 | −1.5275 | −2.0515 |
| CALML5 | −0.8505 | 0.0955 | −2.0375 | −1.8095 | −1.8565 | −0.2835 | 0.1075 | 0.3175 | 0.3015 | 0.9755 | −0.4585 | 0.2435 | −1.0795 | −0.8755 | −0.2945 |
| CaMKIINa [symbol] | −0.206 | −0.14 | −0.255 | 0.249 | 0.184 | −0.287 | 1.136 | 0.966 | −0.702 | 0.057 | 0.586 | 1.009 | −0.628 | 0.657 | −0.297 |
| CAP2 | −1.447 | −0.335 | −0.644 | −0.308 | −1.1 | −0.189 | −1.549 | −1.449 | −0.688 | −1.286 | −1.135 | −1.111 | −0.593 | −1.657 | −1.284 |
| CAP350 | 1.6605 | −1.3245 | −1.2445 | −0.1105 | 0.1285 | 0.2585 | 3.8515 | 1.1315 | 4.1485 | −0.1755 | 1.1595 | −0.5905 | 0.8255 | −0.5605 | 0.3855 |
| CAPN13 | 0.748 | −0.565 | 0.804 | −0.242 | 0.115 | 0.399 | −0.718 | 0.307 | −0.64 | 0.454 | −0.004 | 0.269 | −0.166 | −0.502 | −0.558 |
| CAPN9 | 0.0375 | 0.0865 | 0.0485 | 0.0345 | 0.1935 | 0.4005 | −0.4435 | −0.6676 | 0.2075 | −0.1145 | −0.0345 | −0.4185 | 0.1065 | −0.0795 | −0.0895 |
| CARD14 | 0.1575 | −0.6055 | 0.2915 | −0.2435 | −0.7365 | 1.0725 | −0.7895 | 0.4905 | −0.0955 | −1.1795 | −0.0375 | 2.2475 | 1.4425 | 1.6395 | 0.3445 |
| CART | −0.7595 | −1.0155 | 0.7375 | 0.2895 | −0.5155 | 0.0915 | −0.1265 | 0.1455 | −0.1375 | −0.4765 | 1.1995 | 0.4425 | 0.4835 | 0.4185 | 0.8605 |
| CASP1 | 0.078 | −0.841 | −0.263 | −0.815 | 0.248 | 0.126 | −0.733 | −0.083 | −0.293 | −0.844 | −0.492 | 0.467 | 0.245 | −0.205 | −0.583 |
| CASP3 | −0.429 | 0.039 | −0.226 | −0.437 | 0.011 | −0.078 | −0.217 | −0.036 | 0.12 | −0.049 | −0.188 | 0.142 | −0.316 | −0.071 | 0.13 |
| CASP7 | 0.7645 | 0.4145 | 1.5845 | 0.5645 | 1.8045 | 0.0845 | 0.0845 | −0.0255 | 0.0535 | 0.4745 | 0.2845 | −0.5255 | 0.5795 | 2.6755 | 1.5715 |
| C9LN1 | 0.385 | 0.45 | 1.838 | 0.186 | −0.728 | 0.28 | 0.725 | −0.178 | −0.337 | −0.14 | 0.282 | −0.582 | −0.001 | 0.365 | 1.547 |
| CBR1 | −1.4885 | −0.5455 | −0.9105 | 0.9855 | −0.2115 | 0.1455 | −0.3715 | −0.8565 | −0.1876 | −1.3825 | −0.5775 | 0.3905 | −0.0435 | 0.3855 | −0.7245 |
| CBR3 | 0.125 | 0.228 | 0.091 | 0.054 | 0.196 | −0.224 | −0.53 | −0.453 | 0.29 | 0.216 | 0.214 | −0.714 | −0.069 | −0.915 | −0.21 |
| CBR4 | 1.7475 | 0.7385 | 2.0705 | 1.2025 | −0.4685 | 0.0455 | 0.5275 | 0.1925 | 3.9025 | −0.3485 | −0.0885 | 3.9555 | 2.9235 | 3.4235 | −1.0645 |
| CCL24 | 0.156 | 0.137 | 1.01 | 0.321 | 0.300 | 1.362 | 0.416 | 0.797 | 0.751 | 0.066 | 0.897 | 1.2 | 1.069 | 0.509 | 0.738 |
| CCL29 | −1.515 | 0.19 | −1.76 | −1.976 | −2.995 | −1.629 | 0.545 | 0.852 | −0.458 | 0.407 | −0.457 | −1.285 | −0.996 | −2.468 | −0.009 |
| CCNB2 | 0.8325 | 0.3925 | 1.6845 | 1.1586 | 2.6775 | 0.8975 | −0.2995 | −0.0405 | 1.1015 | 0.5995 | 1.2485 | −0.7815 | 1.7025 | 0.8955 | 0.8195 |
| CCND1 | −1.758 | −1.051 | −1.272 | −0.232 | −0.429 | −0.371 | −0.532 | 0.155 | −0.434 | 1.617 | −0.515 | −0.476 | −1.553 | −0.936 | −0.072 |
| CCNE1 | −1.3245 | −0.1495 | 0.3925 | −0.0025 | −0.4815 | 0.2105 | −1.0045 | −0.3325 | −0.5505 | −0.4365 | −0.7385 | −0.7455 | 0.1045 | −0.3905 | −0.2225 |
| CCNG2 | 0.044 | −0.295 | 1.17 | 0.895 | −0.364 | −0.645 | −0.302 | 0.887 | 0.879 | −0.08 | 0.573 | 0.873 | 1.5 | 1.185 | 0.583 |
| CCNH | −0.3865 | −0.2325 | 0.6035 | 1.1065 | −0.1515 | 0.0225 | −0.6585 | 0.0085 | 0.8995 | 0.2915 | 0.7115 | −0.3805 | 0.5205 | −0.3175 | 0.9155 |
| CD38 | 0.1605 | 0.4205 | 0.3565 | 0.3975 | −0.3655 | −0.4255 | −0.3825 | 0.0055 | 0.3055 | 0.2775 | 0.7525 | −0.5745 | 0.8895 | −0.2485 | 0.1545 |
| C030 | 1.2635 | 0.7565 | 0.9335 | 0.9335 | 0.9585 | 0.5415 | 0.6335 | 0.3245 | 0.3045 | −0.2395 | −0.2965 | 1.7505 | 1.0765 | 1.0745 | 1.3085 |
| CD4 | −0.2225 | 0.6945 | −0.3205 | 0.6655 | 0.0525 | −0.3465 | 0.4375 | −0.2825 | −0.3325 | 0.1285 | 0.0735 | 0.4645 | 0.5125 | 2.1025 | 0.9875 |
| CD58 | 1.353 | −0.245 | 1.356 | 1.492 | 0.999 | 2.459 | 1.368 | 0.127 | 0.303 | 0.052 | 0.789 | 1.038 | 1.038 | 1.707 | 0.624 |
| CD50 | 0.6855 | 0.0305 | 0.8485 | 0.2595 | −0.1555 | 0.6055 | 0.3355 | 0.0795 | −0.0275 | −0.2225 | 0.6955 | 0.2855 | 0.1895 | 0.3135 | −0.1725 |
| CDC2 | 1.5035 | 0.6705 | 2.4305 | 1.3115 | 2.0845 | 1.2375 | 1.4945 | 1.6705 | 0.7285 | 1.4425 | 1.3175 | 1.1755 | 0.8825 | 1.5235 | 1.1045 |
| CDC6 | 0.6485 | 0.3285 | 1.4425 | 0.9505 | 2.0185 | 0.5865 | 0.7805 | 1.2315 | 0.6485 | 0.0515 | 1.9625 | 0.9955 | 0.9295 | 0.4415 | 1.5275 |
| CDCA1 | 1.842 | 1.956 | 1.897 | 1.583 | 2.113 | 2.314 | 3.051 | 1.561 | 2.395 | 2.375 | 2.882 | 3.307 | 1.553 | 2.516 | 1.832 |
| CDCA5 | −0.332 | −1.225 | −0.399 | −1.198 | −0.791 | −0.041 | −0.131 | 0.054 | −1.105 | 0.304 | −0.749 | 0.896 | −0.258 | −1.459 | 0.464 |
| CDCA7 | 2.1355 | 1.1825 | 1.9195 | 2.0695 | 1.5705 | 2.5435 | 2.3475 | 2.1875 | 0.7295 | 0.6075 | 0.8575 | 1.7675 | 2.5975 | 0.7885 | 2.3175 |
| CDH1 | 0.094 | −0.998 | −0.19 | −0.715 | 0.512 | 0.98 | −0.436 | 0.161 | 0.028 | 0.637 | 0.36 | −0.849 | −0.079 | 1.185 | −0.294 |
| CDH3 | 2.7885 | 0.5845 | 2.6875 | 0.2495 | 3.4005 | 0.9175 | −0.4475 | 0.2445 | 1.6785 | −0.2265 | 2.1425 | 1.3385 | 2.0585 | 1.9415 | 0.0085 |
| CDK2 | 0.4245 | −0.3335 | 0.6775 | 0.1785 | 1.8245 | 0.3215 | 0.3065 | −0.0005 | 0.3325 | 1.0145 | 0.0125 | 0.0635 | 0.9085 | 0.0355 | 1.5845 |
| CDKN2A | 1.2155 | 0.1865 | 0.8445 | 1.0215 | 0.8955 | 1.1555 | 1.1925 | −0.0355 | −0.2165 | 0.0045 | 0.3485 | 0.3995 | 0.7895 | 0.3385 | 1.0015 |
| CDKN2D | 0.759 | 0.483 | 0.939 | 1.535 | 0.963 | 1.785 | 1.176 | 0.605 | 0.215 | 1.099 | 1.813 | −0.188 | 0.066 | −0.759 | 1.181 |
| CDKN3 | −0.345 | 0.781 | 0.298 | 0.29 | 0.878 | 1.217 | 0.472 | 1.004 | 0.834 | 0.645 | 0.935 | 0.253 | 0.459 | −1.243 | 0.975 |
| CDT1 | −0.8075 | −1.3225 | −0.1655 | −0.9845 | −0.5195 | −1.5495 | −1.8775 | −0.3605 | −1.4515 | −1.5205 | −2.2885 | −0.8125 | −1.0455 | −1.3215 | −2.0175 |
| CE9PG | 0.188 | 0.424 | 1.518 | 1.519 | 1.423 | 0.104 | 0.318 | 1.176 | 0.414 | 0.726 | 1.281 | 0.484 | 0.228 | 0.195 | 0.804 |
| CELSR1 | 0.198 | −0.285 | 0.817 | 1.224 | 1.304 | 0.424 | 0.02 | 0.569 | 0.544 | 1.122 | 1.065 | 0.007 | 0.464 | 0.284 | 1.014 |
| CENPA | 0.75 | 0.955 | 1.177 | 1.115 | 1.603 | 1.019 | 1.991 | 2.2 | 1.297 | 1.008 | 1.668 | 2.323 | 0.977 | 1.766 | 1.652 |
| CENPE | 0.016 | 0.728 | −0.194 | −0.538 | −0.309 | 0.178 | 0.585 | 0.48 | 0.221 | −0.387 | −0.016 | 0.213 | 0.539 | −0.148 | 0.86 |
| CENPF | 0.2765 | 0.2975 | 0.0585 | 0.0095 | −0.1085 | −0.1245 | 0.1475 | −0.1105 | −0.1545 | −0.0185 | −0.0285 | −0.7455 | −0.8435 | −0.5985 | −0.5405 |
| CENTG3 | −0.6255 | 0.6005 | 0.0185 | 0.0445 | 0.1005 | 0.2125 | 0.0105 | −0.4165 | −0.5305 | 0.2225 | −0.4955 | 0.0905 | −0.5555 | −0.1875 | −1.1395 |
| CES2 | 1.841 | −0.804 | −0.609 | −0.811 | −1.065 | −1.693 | −0.071 | 0.282 | 0.602 | −0.277 | −1.802 | 0.122 | 0.486 | 0.047 | 0.238 |
| CETN3 | −0.3085 | 0.8915 | 0.7715 | 0.2845 | 1.3325 | 2.1125 | 0.4595 | −0.0105 | −0.0645 | −0.5826 | 0.3495 | 1.1445 | 0.2935 | 1.0295 | 0.8575 |
| CGA | −0.4905 | −0.0065 | −0.3475 | −0.4295 | 0.0745 | 0.3945 | 1.8715 | 0.1445 | −0.3795 | 0.5715 | 0.6955 | −0.2255 | 0.4325 | 0.3965 | 0.6545 |
| CGI-12 | −0.9815 | −0.7995 | −0.9235 | 0.9715 | −2.7435 | −0.9865 | 0.4935 | −0.3625 | −1.3905 | −0.9185 | −0.3375 | −1.1095 | −0.0855 | −0.2095 | −0.7735 |
| CGI-49 | 0.011 | −0.174 | 0.872 | 0.185 | 0.821 | −0.772 | −0.02 | 0.293 | −0.409 | −0.072 | −0.071 | 0.196 | −1.045 | −0.687 | −1.058 |
| CGI-4 [symbol] | −0.99 | −1.456 | −0.773 | −0.426 | −0.665 | 0.498 | −0.649 | −1.358 | −0.648 | 0.069 | 0.712 | 0.404 | −1.532 | −0.899 | 0.939 |
| CGN | 0.982 | −0.013 | 1.376 | −0.194 | −0.183 | 0.533 | 1.008 | 0.552 | 0.993 | 0.364 | 1.243 | 1.259 | 0.743 | 2.053 | 1.833 |
| CGNL1 | 0.8545 | −0.0325 | 0.0845 | 0.4085 | 0.7945 | 0.9445 | 0.5875 | 0.5675 | −0.4885 | 0.5365 | 0.5575 | 0.3976 | 1.1245 | 0.5555 | 0.0925 |
| CHAF1B | −0.079 | 0.282 | 0.255 | −0.1 | 0.513 | 0.41 | 0.217 | −0.481 | −0.784 | −0.871 | 0.566 | 0.039 | 0.451 | 0.577 | −0.379 |
| CHC1 | −0.055 | −1.276 | −0.344 | −0.874 | −0.459 | −0.518 | −1.027 | −1.037 | −0.844 | −1.31 | −0.548 | −0.839 | −0.758 | −0.819 | −1.162 |
| CHCHD2 | 1.332 | 2.108 | 2.511 | 3.297 | 1.186 | 2.221 | −0.879 | 2.538 | 0.39 | 2.222 | 1.011 | 3.232 | 1.895 | 1.878 | 0.502 |
| CHCH05 | 0.778 | 1.872 | 1.659 | 1.702 | 3.595 | 4.015 | 1.31 | 0.582 | 0.295 | −0.28 | 0.651 | 4.027 | −0.223 | 3.383 | 0.822 |
| CHI3L1 | −0.288 | 0.625 | 0.843 | −0.285 | 1.784 | 0.211 | 0.802 | 0.848 | −0.348 | −0.444 | 0.074 | −0.013 | 0.713 | −0.227 | −0.557 |
| CHI3L2 | 2.19 | 0.522 | 0.574 | 0.42 | 0.27 | 0.906 | 0.528 | 0.321 | 0.514 | 1.054 | 0.7 | 1.347 | 0.628 | 0.909 | 1.526 |
| CHKA | 4.448 | 0.329 | −0.266 | 0.125 | 5.632 | 2.219 | 0.51 | −0.29 | 0.467 | 0.142 | 0.116 | −0.116 | 0.312 | 3.946 | −0.406 |
| CHCDL | 0.1425 | 0.0395 | 0.6545 | 0.0165 | −0.2015 | −0.2205 | −0.4055 | −0.2925 | −0.1015 | 0.1745 | −0.0845 | −0.5875 | 0.2365 | −0.3055 | 0.1055 |
| CHRDL2 | 0.185 | −0.172 | 0.766 | 1.233 | −0.16 | −0.481 | −0.001 | −0.605 | −0.49 | −0.019 | −0.318 | −0.653 | 0.426 | −0.162 | −0.225 |
| CHST2 | −0.3575 | 0.0135 | −0.3905 | −0.3155 | −0.2755 | 0.1455 | 0.1565 | 0.1965 | 1.0355 | 0.3045 | −0.0975 | 0.5755 | −0.0555 | −0.1355 | −0.1625 |
| CHURC1 | −0.94 | −1.333 | −0.854 | −0.67 | −0.778 | −0.119 | −0.777 | −1.377 | −0.315 | −0.443 | −0.94 | −1.865 | −1.473 | −1.408 | −1.515 |
| CIR | 0.7035 | 1.0335 | 0.5445 | 0.7055 | 0.4395 | 1.0445 | 0.8515 | −0.0355 | 0.0035 | 0.1585 | 0.5275 | 0.2875 | 1.4775 | −0.0955 | 0.7155 |
| CIRBP | −1.2305 | 0.2885 | −0.2875 | 0.3205 | 0.0935 | −0.1015 | 0.3225 | 0.1085 | −0.1745 | −0.9605 | 0.1585 | 0.7375 | 0.5845 | −0.0576 | −1.0546 |
| CKLF | 0.2405 | 1.9225 | 0.4765 | 1.4095 | 1.9765 | 0.4375 | 0.8275 | 0.3175 | 1.2075 | −1.1735 | 1.5065 | 2.0835 | 1.3545 | 0.4325 | −0.8785 |
| CKLFSF6 | −1.3195 | 1.9745 | 1.2076 | 8.8055 | 1.5575 | 1.2625 | 0.2845 | −0.0385 | −2.2945 | −2.0715 | 0.1715 | 0.9495 | 0.0375 | 1.3555 | −1.9295 |
| CKLFSF7 | 0.989 | 0.927 | 1.235 | 1.488 | 1.113 | 0.794 | 0.988 | 1.492 | 1.018 | 0.178 | 0.507 | 1.417 | 0.978 | 0.98 | 0.839 |
| CKMT1 | 1.158 | 0.925 | 2.514 | 1.354 | 0.897 | 1.01 | −0.287 | −0.254 | 1.007 | 0.118 | 0.955 | 0.55 | 1.621 | 0.516 | 0.846 |
| CKS1B | 0.559 | −0.597 | 0.937 | 0.824 | 1.188 | 0.467 | −0.508 | 0.002 | −0.184 | 1.077 | 0.222 | 0.425 | 0.964 | 0.716 | 0.242 |
| CKS2 | 1.9835 | 0.3945 | 0.3905 | 2.2825 | 1.7915 | 1.5375 | 3.1365 | 1.7925 | 0.5945 | −0.6745 | 1.8575 | 1.8825 | 0.7305 | −0.0305 | −0.9975 |
| CL640 | 0.8025 | 0.4435 | 1.5485 | 0.7325 | 0.3585 | 0.4835 | −0.2625 | 0.0575 | −0.6235 | −0.1115 | −0.1565 | 1.5525 | 1.8945 | −1.8765 | 0.0075 |
| CLDN23 | 0.2865 | 0.3955 | 0.8125 | 1.0015 | 0.8985 | 0.3115 | 0.1195 | 0.1185 | −0.4865 | 0.3095 | 0.2105 | 1.0505 | 1.0745 | −0.8955 | 0.3395 |
| CLDN3 | 1.6445 | 2.5395 | 3.1525 | 2.8745 | 2.0676 | 1.9125 | 0.1475 | −0.9225 | 0.5605 | −0.3255 | 1.2015 | 1.2495 | −0.2735 | 0.5315 | 1.2925 |
| CLDN4 | −2.009 | −2.231 | −0.729 | −2.091 | −2.238 | −0.43 | −0.592 | −1.822 | −2.203 | −1.232 | −1.835 | −1.42 | −1.191 | −0.547 | 0.629 |
| CLDN8 | −0.9935 | 0.7805 | −0.6025 | 0.2405 | −0.6645 | 0.0855 | −0.8965 | −0.4975 | 0.0235 | 8.2535 | −0.8805 | −1.1045 | −0.4215 | −0.3535 | −0.3575 |
| CLGN | −0.2785 | 0.1845 | −0.3576 | 0.3125 | 1.1505 | 0.4235 | 0.8525 | 0.6065 | 0.3525 | 0.7045 | 0.3245 | −0.2655 | 0.1035 | 0.2035 | 1.0535 |
| CLIC6 | −0.749 | −0.55 | −0.796 | −0.355 | −0.332 | −0.314 | 0.642 | −0.456 | 0.003 | 0.434 | −0.235 | −0.237 | 0.182 | 1.267 | 0.328 |
| CLNS1A | 0.4345 | −0.3925 | −2.1225 | −0.9755 | −1.6485 | 0.0335 | 0.0545 | −0.7785 | −0.8425 | −2.3375 | −2.8015 | 0.8845 | −2.5635 | 0.7495 | 1.8595 |
| CLTC | −0.47 | 0.728 | 1.537 | 0.061 | 0.522 | 1.146 | −0.314 | 0.335 | 0.738 | 0.544 | 0.818 | −0.357 | 8.368 | 8.235 | 1.217 |
| CLU | 0.414 | 1.261 | 1.104 | 0.917 | 1.58 | 1.85 | 1.175 | 0.798 | −0.06 | 1.332 | 0.572 | 1.555 | 0.83 | 0.97 | 1.3 |
| CMAS | 0.2775 | −0.6065 | −0.2485 | 0.4035 | −1.0525 | 0.3225 | 0.2915 | 0.0145 | −0.8135 | 0.1185 | −0.9715 | 0.1045 | 0.0655 | 0.1375 | −0.5025 |
| CML65 | −0.203 | −0.231 | 0.325 | −0.125 | 0.704 | 0.184 | 0.109 | −0.219 | −0.187 | −1.048 | 0.742 | 8.295 | −0.074 | 0.299 | −0.194 |
| CNIH | 0.066 | −0.968 | −0.037 | −0.907 | −0.523 | −0.446 | −0.444 | 0.888 | 0.182 | 0.277 | 0.381 | −0.476 | 0.051 | −0.182 | −0.188 |
| CNOT10 | 0.8895 | −0.8095 | −0.0885 | −0.2045 | −0.1195 | 0.0435 | 0.8575 | 0.2795 | 0.8485 | 0.8095 | 0.9105 | 0.0005 | −0.5265 | 0.7595 | 0.6285 |
| CNOT2 | 0.096 | 0.224 | 0.639 | 0.517 | −0.043 | 0.318 | −0.026 | −0.185 | 0.877 | −0.007 | −0.174 | −0.217 | −0.269 | 0.818 | 0.178 |
| CNOT7 | −2.574 | −1.614 | 1.058 | −1.965 | −2.689 | 0.337 | −2.355 | 0.371 | −1.983 | −1.769 | −2.064 | −1.352 | −1.016 | −1.831 | −1.722 |
| CNP | −0.5315 | −0.4435 | −0.0655 | −0.6125 | −0.3165 | −0.2485 | −0.8465 | −1.0985 | 0.2445 | −0.4175 | 0.6485 | −1.2385 | −0.2835 | 0.2945 | −1.0295 |
| CNTNAP2 | 0.195 | 2.578 | 0.239 | −0.107 | 0.052 | 0.365 | 0.513 | 0.425 | 2.177 | 1.453 | 0.853 | −0.137 | −0.286 | 0.303 | −0.102 |
| COASY | −0.5275 | 1.4965 | −0.5455 | −0.1245 | −0.4305 | −0.6375 | 1.6745 | 1.4315 | 2.0015 | 2.5805 | 2.0945 | 0.6715 | 0.3535 | −0.4055 | 2.3095 |
| COL11A2 | 0.253 | −2.526 | −2.22 | −2.478 | −0.701 | −0.31 | 0.763 | −1.225 | −0.78 | −0.058 | −3.401 | −2.266 | −0.598 | −1.803 | −2.252 |
| COL27A1 | −0.051 | 4.15 | −0.311 | −0.041 | −0.245 | 0.569 | 4.36 | 0.212 | 0.522 | 6.321 | 6.07 | 0.004 | −0.374 | −0.114 | −1.491 |
| COL4A5 | −0.7825 | 0.9105 | 1.7115 | 4.0455 | −1.2175 | 1.4805 | 4.6135 | 2.7835 | 4.8755 | 3.5545 | 2.2225 | 1.8855 | 2.9685 | 3.3895 | 8.3675 |
| COL8A1 | −0.565 | −0.276 | −0.288 | −0.133 | −0.873 | −0.08 | −0.38 | −0.505 | −0.569 | −0.044 | −1.39 | −0.344 | 0.334 | 0.095 | −0.23 |
| COL8A3 | 0.467 | 0.503 | 0.543 | −0.503 | 1.08 | 0.657 | 0.331 | −0.315 | 0.062 | −0.404 | −0.629 | 0.495 | −0.243 | −0.191 | −0.158 |
| COMMD1 [symbol] | 0.5275 | 0.3615 | −0.0745 | −0.1835 | −0.1355 | −0.0185 | −0.3265 | −0.2295 | 0.5795 | 0.2345 | −0.0555 | −0.2275 | 0.1175 | 0.0545 | −0.2155 |
| COMMD5 | −0.3685 | −0.2125 | −0.4005 | −0.2585 | −0.4985 | −0.2085 | 0.0115 | −0.1485 | −0.2895 | −0.0635 | −0.8895 | −0.3465 | 0.3085 | −0.4465 | −0.3795 |
| COPS7B | 0.382 | −0.173 | 8.478 | 0.028 | −0.112 | 0.789 | −0.617 | −0.3 | −0.814 | −0.416 | 0.27 | −8.338 | 0.118 | −0.129 | 0.621 |
| CORO6 | −0.2925 | −0.1605 | −0.8285 | −1.2225 | 0.0135 | 0.1645 | −0.1405 | −0.0005 | 0.1015 | −0.4895 | −0.2255 | 0.2565 | −0.3035 | −0.0845 | −0.4255 |
| CORT | 0.177 | 0.463 | 0.358 | 0.583 | −0.752 | 0.465 | 0.297 | −0.264 | −0.407 | −0.014 | 0.457 | 1.17 | 0.635 | 0.498 | 0.021 |
| COX17 | 0.385 | 0.584 | 0.53 | 0.665 | −0.535 | 0.538 | 0.338 | −0.014 | 0.065 | 0.217 | 0.523 | 1.261 | 0.376 | 0.514 | 0.04 |
| COX5A | 0.885 | −0.507 | −0.268 | 0.448 | −0.84 | 0.08 | −0.446 | −0.964 | −0.289 | 0.228 | 0.175 | −0.097 | 0.236 | 1.032 | 0.322 |
| COX6A | 0.1485 | 0.4985 | 0.7815 | −0.0445 | 0.2865 | 0.8455 | −0.4195 | −0.9375 | −1.8025 | −1.1565 | −0.5445 | −0.1465 | −1.5755 | −0.2565 | −1.0495 |
| COX8A1 | 0.3285 | −0.3945 | 0.0325 | 0.3675 | −0.3795 | −0.0705 | 0.2415 | −0.0885 | −0.1865 | −0.3335 | 0.9115 | 0.2765 | 0.3016 | 0.8555 | −0.5795 |
| COX8C | 2.4445 | 1.0115 | −0.2475 | 0.6625 | −1.5045 | 1.5025 | 5.8935 | 6.5545 | 1.8685 | 3.5775 | 2.5355 | 1.5015 | 2.3035 | 0.4785 | 0.6875 |
| COX7A2 | 0.426 | −1.729 | 0.98 | 1.246 | 8.89 | 1.116 | 0.58 | 0.579 | 1.188 | −0.616 | −0.28 | 1.335 | 1.761 | 0.434 | −0.968 |
| CP | −0.4225 | −0.9305 | −0.4315 | −0.0485 | −0.3735 | 0.0335 | −0.4405 | −0.4405 | 0.0025 | −0.1885 | −0.3495 | −0.5965 | 0.1085 | −0.3975 | −0.1905 |
| CPAMD0 | −0.257 | −0.737 | −0.862 | −0.883 | −0.17 | −0.358 | −0.234 | −0.054 | −1.192 | −0.785 | −0.433 | −0.252 | −3.602 | −0.595 | −1.378 |
| CPB1 | −0.5725 | −1.4835 | −0.6135 | −0.0095 | −1.5285 | −0.2265 | −0.4725 | −0.5215 | −0.9005 | −0.9005 | −1.1005 | 0.9115 | −0.5345 | −0.4305 | −1.1795 |
| CPEB3 | −0.9 | 0.01 | −0.01 | −1.149 | 0.145 | −0.244 | 0.908 | 0.642 | 0.138 | 8.083 | 1.281 | −0.417 | 0.2 | 0.449 | 0.449 |
| CPEB4 | 2.439 | 1.782 | 1.58 | 2.228 | 0.92 | 1.704 | 0.356 | −0.036 | −0.253 | 1.388 | 1.578 | 0.74 | 0.834 | −0.043 | 0.085 |
| CPSF3 | −1.4985 | −1.7735 | −1.8835 | −1.6015 | −1.1785 | −0.8905 | −0.7046 | −1.3385 | −1.4835 | −1.8505 | −1.1265 | −0.9605 | −2.1585 | −0.5826 | −1.4425 |
| CRABP1 | −0.355 | −0.922 | −0.623 | −1.772 | −2.026 | −0.706 | −2.155 | −2.603 | −2.223 | −2.047 | −2.175 | −3.287 | −2.411 | −1.672 | −2.314 |
| CREB3L4 | 1.405 | 0.183 | 0.132 | 0.035 | −0.105 | 0.813 | −0.449 | −0.358 | 1.583 | 1.102 | 1.595 | −0.427 | 0.127 | 1.158 | 1.08 |
| CRIP1 | −1.509 | −0.429 | 0.802 | 0.668 | 0.072 | 0.044 | −0.199 | −0.127 | −0.384 | −0.621 | −0.428 | −0.276 | −0.559 | −0.88 | −0.713 |
| CRYBB2 | 0.258 | 0.225 | 1.461 | −0.195 | 0.438 | −0.958 | 0.838 | 0.414 | 0.309 | 0.119 | −2.469 | 1.192 | 0.451 | −0.852 | 0.009 |
| CRYL1 | 0.757 | 1.21 | 0.579 | −0.477 | −0.417 | 0.307 | 0.292 | 1.262 | 0.487 | 1.26 | 0.908 | 0.943 | −0.421 | 0.105 | −1.049 |
| CRYZ | −0.2335 | 0.1905 | 0.5295 | −0.4555 | 0.8735 | 0.9705 | 0.4075 | 0.3076 | 0.9605 | 0.2455 | 0.3375 | 0.2115 | 1.0775 | 0.4745 | 0.9555 |
| CSDA | 0.2855 | 0.3015 | 0.3285 | 0.0195 | −0.2175 | 0.0885 | −0.4095 | −0.1715 | −0.2165 | −0.0375 | 0.6305 | −0.3965 | 0.0665 | −0.2916 | −0.2155 |
| CSE1L | 1.0455 | 0.8485 | −0.4855 | −0.1865 | −1.2615 | 0.4075 | 0.3355 | 0.8765 | 0.2855 | 1.0545 | 0.4705 | 0.0215 | −0.4685 | −0.8455 | −0.0985 |
| CSK | 0.9055 | −0.2875 | 0.4145 | 2.0735 | −1.3215 | 2.2225 | 1.8655 | 2.2005 | 0.8335 | 1.7525 | 0.9205 | −2.2455 | 0.7015 | 1.2165 | 3.3945 |
| CSRP1 | 1.2905 | −0.0425 | 1.1785 | −0.3705 | −1.1925 | −0.1675 | −0.0515 | −0.5885 | 0.8935 | −0.5095 | 0.2295 | −0.8025 | 1.1055 | −1.1175 | −1.4705 |
| CSRP2 | 2.042 | 0.574 | 2.296 | 0.498 | 1.653 | 0.806 | 0.505 | 1.371 | 0.214 | 0.105 | 0.028 | 1.779 | 1.473 | 3.027 | 0.739 |
| CST5 | −0.7205 | −0.7705 | −1.1885 | 6.8355 | 0.0225 | −0.2575 | 2.0205 | −0.3815 | −0.1445 | 0.8325 | −0.6855 | 0.1855 | 6.6425 | −0.0825 | 0.4665 |
| CSTB | 0.232 | −0.139 | −0.55 | −0.821 | 1.025 | −0.475 | −0.581 | 0.183 | 0.601 | 0.39 | 0.588 | −0.482 | −0.07 | −0.537 | 0.946 |
| CTAG1B | −0.2375 | −1.4435 | −0.6155 | −0.7515 | −0.8055 | −0.6495 | 0.2615 | 0.4055 | −2.3945 | 8.1455 | −0.2795 | −0.0585 | −0.8065 | 0.1795 | −0.9875 |
| CTBP2 | 0 | 0.138 | −0.603 | −0.348 | −1.350 | 0.912 | 0.321 | −0.542 | −0.233 | 0.17 | 0.879 | 1.823 | −0.353 | −1.618 | 0.184 |
| CTNNA1 | 0.562 | −1.389 | −1.806 | −1.075 | −1.228 | 0.543 | 0.462 | −0.815 | −0.055 | 1.956 | 0.794 | −1.175 | −0.314 | −1.299 | −1.531 |
| CTNNAL1 | 1.4265 | 0.9065 | 1.3405 | 0.9865 | 1.0945 | 8.5495 | 0.5105 | 1.0655 | 0.3895 | 0.7175 | 1.7075 | 1.0435 | 0.8265 | 0.8785 | 0.7515 |
| CTNND2 | 5.725 | 2.379 | 3.183 | 1.152 | 2.987 | 2.118 | 0.347 | 0.311 | 2.038 | 1.881 | 1.019 | 1.57 | 3.281 | 2.337 | 0.514 |
| CTPS | 0.372 | 0.724 | 2.204 | 1.244 | 0.145 | 0.574 | −0.134 | 1.124 | 0.787 | −0.656 | 0.947 | 1.847 | 1.146 | 1.272 | 0.959 |
| CTSL2 | −0.3125 | 0.2925 | −0.8465 | −0.1195 | 1.4975 | 0.7335 | 0.1495 | 0.3995 | 0.1925 | −0.4225 | −0.4755 | 0.4205 | 0.8745 | 0.0375 | 0.5075 |
| CTSS | 0.96 | −0.47 | 0.04 | 1.81 | −0.83 | 0.848 | 1.24 | −0.18 | 0.985 | 0.96 | 1.38 | 2.32 | 1.677 | −0.01 | 0.159 |
| CTTN | 0.1635 | −0.3545 | 3.0095 | 2.8725 | −0.8765 | 1.5755 | 1.5255 | 2.0495 | 0.2025 | 0.0475 | 3.8455 | 1.2485 | 1.4705 | −1.3425 | 1.4825 |
| CX3CL1 | −0.2885 | −1.0016 | −0.3835 | 0.2535 | 0.8595 | 0.2745 | −1.5335 | −0.4005 | 0.1385 | −0.4015 | −0.9645 | 0.5195 | −0.4385 | 0.5225 | −0.9505 |
| CXCL9 | −1.8525 | −0.6595 | −2.4565 | −1.7605 | −2.7155 | −0.1576 | −1.3815 | 0.1935 | −1.0665 | 1.4985 | 1.4325 | −1.3675 | −2.2116 | −0.2005 | 2.1095 |
| CXorf39 | −0.642 | −1.366 | −0.502 | −1.257 | −1.249 | −2.015 | −1.658 | −1.672 | −1.567 | −1.831 | −2.264 | −2.177 | −1.098 | −0.009 | −0.707 |
| CXXC4 | 0.234 | 0.897 | 0.143 | −0.437 | −0.214 | −0.291 | −0.852 | −0.951 | −0.61 | 0.19 | 0.282 | −0.612 | −1.085 | −0.574 | 8.078 |
| CYB5 | 1.3775 | 1.7415 | 1.5785 | 1.1095 | 1.0855 | 1.1885 | 0.8315 | −0.9155 | −0.8955 | −0.5885 | −0.5025 | 0.4625 | −0.3105 | 0.8445 | 0.2925 |
| CYB561 | 0.067 | 0.726 | −0.579 | −0.28 | 0.815 | 0.634 | 0.034 | −1.453 | −1.298 | −0.398 | 0.015 | −0.942 | −0.803 | −0.847 | −0.21 |
| CYC1 | 0.075 | −0.054 | 0.155 | −0.082 | 0.016 | −0.015 | 0.029 | 0.089 | −0.037 | −0.077 | −0.008 | 0.245 | 0.543 | −0.253 | 0.135 |
| CYHR1 | −0.6355 | −1.3835 | 1.0375 | 1.9045 | −1.3225 | −0.3745 | −0.4335 | −1.2995 | −1.0775 | −0.8645 | 1.2345 | 0.1575 | −1.0075 | −0.3195 | −0.6635 |
| CYP2A13 | −0.191 | 1.003 | 1.271 | 0.877 | 0.216 | 0.764 | 0.772 | 0.729 | 0.343 | 0.003 | 0.776 | 0.814 | 0.422 | 0.504 | −0.071 |
| CYP4X1 | −1.3776 | −2.2015 | −2.7055 | −0.8535 | −1.9355 | 0.1485 | −2.0885 | −1.8805 | −2.0675 | −2.0585 | −1.3205 | −2.1045 | −1.7095 | −1.8435 | −1.7765 |
| D15Wsu75 | −1.0675 | −0.7705 | −1.0185 | −0.7055 | −0.9375 | 0.0415 | −1.2175 | −1.3225 | −1.1575 | −0.8635 | −0.6825 | −1.4585 | −0.9515 | −1.1205 | −0.0665 |
| DACH1 | −1.771 | −1.205 | −1.469 | −0.866 | −1.588 | −0.528 | −2.096 | −2.11 | −1.459 | −1.906 | −1.112 | −2.242 | −1.467 | −2.022 | −1.953 |
| DACH1 | 0.347 | −0.107 | 0.24 | 0.161 | 1.008 | 0.058 | 0.842 | 0.52 | 0.088 | 0.004 | −0.288 | 0.835 | −0.124 | 1.437 | 0.478 |
| DACH1 | 0.877 | 0.465 | 1.64 | 1.351 | 0.452 | −0.422 | 0.363 | 1.01 | −0.242 | 0.219 | 1.073 | 3.01 | 0.109 | 1.129 | 0.928 |
| DAP3 | −0.6995 | −0.6595 | −0.5995 | −0.7935 | −0.8405 | 0.3025 | −0.2185 | 0.1725 | 0.0395 | −0.6745 | −0.9195 | 0.2695 | 0.0145 | 0.2485 | −0.4865 |
| DAPP1 | −0.058 | −0.114 | −0.083 | 1.041 | 0.502 | 1.046 | 0.425 | 0.039 | −0.841 | 1.26 | 1.062 | 0.669 | −0.371 | −0.105 | 0.251 |
| DAZAP2 | 0.588 | −0.468 | −0.688 | −0.847 | 0.14 | 0.384 | 0.252 | 0.278 | 0.634 | 0.798 | 0.724 | 0.946 | 0.52 | 0.069 | 0.193 |
| DBI | 0.7595 | 0.5725 | 0.3635 | 0.2995 | 0.6835 | 0.9025 | 1.3115 | 0.2725 | 0.3895 | 0.4465 | 0.4515 | 0.8775 | 0.7635 | 0.5155 | 1.4615 |
| D9N1 | 0.563 | 1.588 | −0.126 | 0.638 | 1.081 | 1.144 | 1.195 | 0.582 | 0.385 | 0.956 | 1.082 | 0.51 | 0.291 | 1.439 | −0.245 |
| DC13 | 1.1745 | −0.8785 | 0.3935 | −0.3355 | −0.4495 | 0.0415 | 0.3855 | 0.8015 | −0.3945 | 0.1165 | 0.0915 | −0.0255 | −0.2425 | 0.5155 | 0.6915 |
| DCPS | 0.9876 | 0.3295 | 0.1485 | 0.0045 | −0.3985 | −0.4375 | −0.1765 | −0.6135 | −0.3915 | 0.5125 | 1.0715 | 0.0015 | −0.6735 | 0.8185 | −0.0045 |
| DDHD2 | 0.6816 | 0.3805 | 0.8025 | 0.4315 | 0.9555 | 0.4305 | 0.1245 | −0.8015 | 0.2015 | 0.3075 | 0.2035 | −0.5335 | −0.2115 | 0.7495 | 0.8975 |
| D0X17 | 0.7085 | −0.4285 | 0.9005 | −0.1085 | 0.4805 | 0.2885 | 0.5805 | 0.5355 | −0.0245 | −0.1845 | 0.0585 | 0.3795 | −0.3845 | 1.4845 | 0.2955 |
| DDX39 | 1.021 | 0.844 | 0.439 | 1.007 | 1.838 | 0.401 | −0.886 | −0.497 | 0.78 | 0.197 | −0.481 | 0.549 | 0.787 | 1.858 | 0.582 |
| DDX48 | 0.1725 | −0.5155 | −0.0815 | −0.3075 | −0.4045 | 0.1615 | −0.4185 | 0.4455 | 0.3335 | −0.3025 | 0.8015 | −0.3945 | 0.2305 | −0.5165 | 0.5915 |
| DDX58 | 1.5435 | 0.6695 | 0.4165 | 1.0075 | 0.0605 | 0.9265 | 1.4795 | 1.7475 | 1.4275 | 0.8405 | 1.8565 | 1.7695 | −0.3966 | 0.7125 | 1.2855 |
| DEF6 | 1.016 | 1.393 | 0.715 | 2.348 | 1.538 | 0.633 | 0.799 | 0.768 | −0.077 | 1.339 | 1.594 | 1.264 | 1.584 | 0.704 | −0.013 |
| DEK | −0.148 | −1.313 | −1.455 | 0.702 | 0.970 | 1.415 | 0.405 | −0.294 | −0.139 | −0.393 | −1.555 | 0.545 | 0.162 | 0.302 | 0.2 |
| DHCR7 | 0.958 | 0.653 | 1.165 | 1.423 | 0.561 | 1.114 | −0.205 | −0.155 | 0.005 | 0.158 | −0.506 | −0.078 | −0.465 | 1.018 | −0.87 |
| DHRS7 | −1.1585 | −0.4275 | 0.0245 | −0.5545 | 0.1575 | −1.2345 | 0.8145 | 0.4855 | −1.9325 | −1.8495 | 0.1665 | −1.6995 | −1.0535 | 0.6625 | −1.3775 |
| DHX57 | −1.2925 | −1.1305 | −1.5535 | −0.1795 | −0.3715 | −0.1785 | −1.4435 | −2.0885 | −1.7185 | −2.0065 | −1.8745 | −0.9815 | −1.4505 | −1.1085 | −1.8825 |
| DIRC1 | −0.084 | 0.392 | −0.589 | −0.78 | −0.489 | −0.117 | −0.824 | −0.244 | −0.289 | −0.082 | 0.25 | −0.485 | −0.275 | −0.317 | 0.19 |
| dJ222E13 | 2.247 | 1.272 | 1.288 | 2.677 | 2.758 | 1.639 | −1.251 | −0.341 | 1.518 | 2.056 | 3.295 | 0.224 | 2.358 | 2.614 | 4.085 |
| DKFZP434 | 0.085 | −0.878 | 0.585 | 0.273 | −0.201 | 0.509 | 0.047 | 0.137 | 0.717 | −0.285 | 0.126 | 0.958 | 0.873 | 0.223 | −0.3 |
| DKFZp434 | 0.2325 | −0.7795 | −0.2445 | 0.5545 | 1.0185 | −0.2315 | 0.4315 | 1.1115 | 1.3375 | 0.6625 | 0.7876 | 0.2805 | 1.6215 | 0.9595 | 0.5495 |
| DKFZP434 | −0.487 | −0.46 | −0.269 | −0.158 | 0.727 | −0.19 | 0.038 | −0.22 | −0.175 | −0.382 | −0.302 | −0.965 | 0.234 | −0.989 | −0.811 |
| DKFZp434 | −0.8195 | −0.8125 | −0.4565 | −0.3705 | −0.3605 | −0.1355 | −0.9995 | −0.5295 | −0.3005 | −1.2475 | −0.9815 | −0.6805 | 0.0935 | −0.2045 | −0.7055 |
| DKFZP564 | −0.0525 | −0.1325 | 0.6405 | 0.1075 | 0.4575 | 0.2595 | −0.8125 | −0.0885 | 0.7345 | 0.1135 | 0.4965 | −0.3115 | 0.2445 | 0.3165 | 0.7775 |
| DKFZp564 | 0.698 | 1.48 | 1.594 | 0.906 | 2.012 | 2.175 | 0.94 | 0.214 | 0.301 | 1.084 | 0.867 | 0.654 | 0.295 | 0.571 | 1.189 |
| DKFZP564 | −0.8835 | −0.5965 | −0.0935 | −0.0485 | 0.2645 | −0.4925 | 0.4945 | 0.3025 | −0.4215 | −0.0885 | −1.0145 | 0.2435 | 1.5755 | 0.4395 | −0.2485 |
| DKFZP564 | 0.139 | 0.373 | 0.293 | 1.010 | 0.455 | −0.074 | 1.343 | 1.016 | 0.515 | 0.449 | −0.63 | 0.805 | 0.966 | −0.134 | −0.013 |
| DKFZP564 | 0.849 | −1.319 | −0.366 | −0.488 | −1.121 | −0.393 | 0.289 | 0.012 | −0.321 | 1.113 | −0.645 | −0.6 | −0.867 | −0.588 | 0.654 |
| DKFZp586 | −0.9415 | −0.2335 | −0.8625 | −0.4555 | −0.0625 | −0.8615 | 2.1525 | −0.7975 | −0.1245 | −0.3255 | −1.7305 | −0.8685 | −0.3485 | 0.1705 | −0.7185 |
| DKFZp761 | 0.128 | 1.31 | 0.477 | −0.18 | 0.001 | 0.464 | 1.41 | 0.418 | −0.22 | 2.452 | 2.819 | −0.231 | −0.107 | −0.223 | 0.111 |
| DLK1 | −0.198 | −2.497 | −1.791 | −0.335 | −0.431 | −0.206 | −1.48 | −1.88 | −1.029 | −1.137 | −0.935 | 1.14 | 0.236 | −0.008 | −1.413 |
| DLX5 | −0.2465 | −1.3205 | −0.3255 | −0.2005 | −0.8825 | 0.4105 | −1.1935 | −0.9715 | −0.6245 | −0.5465 | −0.5485 | −0.8535 | −0.8055 | −0.2965 | −0.9775 |
| DNAJA4 | −2.7275 | −3.7045 | −3.8285 | −3.3955 | −2.6865 | −0.9065 | −2.9235 | −2.9045 | −1.7495 | −3.0055 | −2.8065 | −3.6555 | −2.2805 | −2.4385 | −2.4785 |
| DNAJC1 | 0.002 | 0.286 | −1.691 | 0.455 | 0.266 | −0.155 | 0.303 | 0.514 | 0.031 | 0.164 | 0.178 | 0.749 | −0.088 | −1.554 | 0.534 |
| DNAJC12 | −3.28 | −2.59 | −4.122 | −2.79 | −3.171 | −0.357 | −2.229 | −2.519 | −4.222 | −2.321 | −0.481 | −3.411 | −2.428 | −3.21 | −2.87 |
| DNAJD1 | 1.126 | −0.053 | 1.116 | 0.5 | 0.639 | 0.826 | 0.908 | 0.009 | 0.269 | 0.144 | 1.103 | 0.042 | 0.6 | −0.16 | 0.476 |
| DNALI1 | −0.683 | −0.419 | −1.106 | 0.494 | −0.409 | −0.767 | −0.382 | −0.293 | 0.2 | −0.002 | −1.151 | −0.308 | 0.623 | −0.157 | 0.059 |
| DNMT1 | 4.2025 | 1.3285 | 2.1045 | 0.9575 | 0.7565 | 2.4235 | 1.9915 | 0.6965 | 1.7175 | 2.9035 | 1.4255 | −0.1235 | 2.3045 | 3.0095 | 0.8135 |
| DOX1 | 1.1765 | 0.6685 | −0.4185 | 0.4015 | −2.0865 | −0.9125 | 0.0545 | −0.0915 | 0.5185 | 0.6365 | 0.5885 | 0.1675 | 1.0965 | 0.5205 | 0.2205 |
| DSCR1 | 1.0925 | 0.4635 | −0.9495 | −0.0035 | 0.6025 | −0.0715 | 0.3955 | 1.2325 | 0.2405 | 1.3585 | 1.1975 | 0.8885 | −1.1615 | 0.6645 | 0.7485 |
| DSCR1L2 | −0.8925 | 0.4075 | 0.4095 | 0.0845 | 0.1985 | 0.2105 | 0.1975 | 0.3295 | 0.3795 | 0.2015 | −0.2345 | 0.0055 | 0.0355 | 0.5225 | 0.7595 |
| DSP | 0.7585 | −0.2795 | −0.5015 | 0.7455 | −0.0185 | 0.5525 | 0.6985 | −0.0195 | 0.2515 | −0.2625 | 0.0155 | 0.2115 | −0.9045 | 0.4685 | −0.5585 |
| DTYMK | 0.8535 | −0.5735 | −0.6165 | −0.5285 | −0.8105 | −0.8135 | −0.6225 | −0.7825 | −1.0075 | 0.0315 | −0.3975 | −0.4505 | −0.5775 | 0.8405 | −0.7805 |
| DUSP23 | −1.6975 | −1.8135 | −1.1995 | −0.2955 | −1.2675 | 0.3145 | −0.8905 | 0.0115 | −0.7935 | 0.6215 | −0.5905 | −2.4885 | −0.1235 | −1.1035 | −0.9515 |
| DUSP4 | 0.4195 | 0.4955 | −0.5085 | −0.4345 | 0.0495 | −0.2335 | −0.5155 | −0.4635 | 0.2085 | −0.6455 | −0.8105 | −0.1455 | 0.3615 | 0.2355 | 0.5885 |
| DUSP5 | −0.266 | 0.218 | 0.065 | 0.085 | −0.158 | −0.188 | −0.071 | 0.249 | 0.189 | 0.08 | 0.094 | −0.15 | 0.093 | 0.149 | −0.054 |
| DVL1 | 0.204 | −0.27 | 1.163 | 0.024 | 0.668 | −0.307 | −0.712 | −0.145 | 0.23 | 0.489 | −0.187 | −0.75 | 0.039 | −0.451 | 1.275 |
| DXYS155 [symbol] | 0.534 | 0.464 | 0.596 | 0.057 | 1.132 | 1.248 | 0.681 | 0.069 | −1.109 | 0.668 | 0.178 | 0.986 | 0.334 | 0.1 | 0.824 |
| DYRK4 | −0.2845 | −0.0035 | 1.1205 | 0.0745 | −0.2885 | 0.7345 | 0.1125 | −0.1185 | 0.1825 | −0.6215 | −0.1935 | −0.1005 | 1.0855 | −0.1895 | 0.1685 |
| o(y)2 | −0.823 | −0.394 | −0.599 | −0.628 | −0.181 | −0.301 | −0.096 | −0.624 | 0.606 | 0.089 | 0.086 | −0.987 | −0.326 | −0.42 | 0.007 |
| EAF2 | −1.0725 | 0.3915 | 0.5955 | 1.5805 | −0.0495 | 0.1945 | 0.9305 | 0.2625 | 1.4755 | −0.6325 | 1.4215 | 3.0665 | 0.8005 | 1.7095 | 1.1775 |
| EAP30 | 0.9745 | 0.6395 | 1.3975 | 0.2975 | 1.2775 | 0.5505 | −0.8625 | −0.6355 | 0.6745 | 0.7035 | −0.0775 | 1.2355 | 1.6285 | 1.8825 | 0.7855 |
| ECHDC1 | −0.6155 | −0.5955 | 1.5135 | −0.6825 | −1.9205 | −1.0105 | −0.2375 | −0.4455 | −0.9105 | −0.2365 | −1.5045 | 0.0205 | −0.7785 | −0.7485 | −0.7295 |
| ECHDC3 | −0.3595 | −1.2125 | −1.1605 | −1.4035 | −1.5285 | −1.0555 | 0.1815 | −0.0055 | 0.8845 | −0.4725 | −0.5005 | −1.5125 | −0.6125 | −0.2545 | −0.3825 |
| EEF1A2 | 1.144 | 0.7 | 0.272 | 0.092 | 0.527 | 0.871 | 0.525 | 0.223 | 0.23 | 0.092 | 1.187 | −0.121 | −0.183 | 0.93 | 0.409 |
| EFHC1 | 0.1815 | 0.2305 | 0.7755 | −0.1905 | 1.4035 | 0.9025 | 0.2425 | −1.0205 | 0.3455 | 0.6505 | 1.0075 | −0.3535 | −1.1115 | −0.5745 | −0.2535 |
| EFNA4 | 0.7795 | 0.8975 | 1.4015 | 0.6405 | 1.7075 | 0.6415 | 0.8975 | 0.0905 | 0.3895 | 0.1945 | 0.1355 | 0.7385 | 0.8025 | 0.2855 | 0.4285 |
| EFS | 0.3345 | −0.7475 | −0.4055 | 0.2795 | 0.3575 | 0.3085 | −0.1215 | 0.3875 | −0.2385 | 0.0715 | −0.1845 | 0.3845 | −0.1405 | 0.1695 | 0.7095 |
| EIF2C2 | 1.3795 | −0.8715 | 1.9345 | −0.0915 | 0.4655 | −0.1085 | −0.4715 | 1.0395 | −0.8755 | 0.0525 | 0.5095 | 1.1475 | 0.0985 | 0.1605 | 0.5765 |
| EIF2C4 | −0.656 | 0.02 | 0.508 | 0.071 | 0.139 | 0.101 | −1.005 | −0.449 | 0.302 | 0.534 | 0.284 | −0.461 | 0.453 | −0.003 | 0.832 |
| EIF4EBP1 | −0.6455 | −0.3965 | −1.0845 | −0.2265 | 0.3455 | 0.2045 | −0.5135 | 0.0295 | −0.9685 | −0.9245 | 0.3335 | −0.4745 | −0.3225 | −0.0855 | −0.2765 |
| EIF4G1 | 0.766 | −0.239 | 0.517 | 1.292 | 0.358 | 0.781 | 0.628 | 0.69 | 1.169 | 0.337 | −0.116 | −0.99 | 0.229 | 0.514 | 1.538 |
| EIF4G3 | −0.215 | −0.88 | −0.778 | −0.62 | −0.855 | −0.353 | 0.329 | 0.051 | −0.055 | −0.381 | −1.257 | −0.312 | 0.381 | 0.202 | −0.009 |
| ELAC1 | 0.128 | 1.349 | 0.229 | 1 | 1.127 | 0.311 | −0.03 | −0.995 | −0.781 | 0.103 | 0.873 | 0.04 | −1.375 | 0.134 | 0.439 |
| ELL2 | −1.5665 | −1.1335 | −2.2925 | −0.3535 | −1.3295 | −0.3795 | −0.0495 | −0.7175 | −2.2285 | −1.7625 | −1.9325 | −0.5925 | −0.6095 | −0.8885 | −0.6005 |
| ELL3 | 1.3575 | 2.4355 | 2.8805 | 3.3105 | 3.1945 | 3.2555 | 2.8315 | 2.8135 | 1.0805 | 3.0475 | 4.7355 | 2.0375 | 2.0225 | 0.1885 | −0.0255 |
| ELOVL5 | −0.0045 | 0.4735 | 0.2185 | 0.0395 | 0.4495 | −0.4045 | 1.1025 | 0.4535 | 1.0695 | 0.6045 | 0.3895 | 0.6625 | 0.0655 | 0.9195 | 0.4605 |
| EN1 | −0.5195 | 0.0175 | 0.0205 | −0.6195 | −1.1645 | 0.2985 | 0.9016 | −1.9815 | −1.3295 | −1.2775 | −0.0905 | 0.4045 | −1.3415 | 0.1495 | 0.8365 |
| ENAH | −1.2295 | −1.8585 | −2.3385 | −0.9545 | −1.2025 | −0.2305 | −0.9075 | −0.4605 | −0.4125 | −0.9285 | −2.2735 | −1.1685 | −0.8665 | −0.3895 | −1.1785 |
| EPHX2 | −0.970 | −0.922 | −0.741 | −1.02 | 0.686 | −1.317 | −0.105 | −1.971 | −0.914 | −0.232 | −0.392 | −0.291 | −1.509 | 0.598 | −0.852 |
| EPLIN | −1.0125 | −0.6225 | −1.0105 | −0.5665 | −0.1295 | −0.0855 | 0.2245 | −0.1115 | −0.2055 | −0.2195 | −0.7755 | −0.2285 | 0.1345 | −1.2745 | −0.7485 |
| EPN3 | −1.6465 | −1.7095 | −0.9185 | −2.2615 | −2.4955 | −0.8475 | −0.7245 | −0.3185 | −1.4085 | −1.0215 | −1.2625 | −2.3195 | −2.4025 | −0.9285 | −9.8915 |
| EPOR | 0.916 | −0.106 | −0.397 | 0.669 | −0.288 | 0.451 | 1.189 | 0.741 | 0.104 | 0.12 | 0.045 | 0.139 | 0.739 | 1.872 | 0.996 |
| ERBB2 | −0.9335 | −6.0925 | −2.2575 | −4.9245 | −4.8575 | −2.9705 | −4.8415 | −4.4375 | −2.1685 | −5.3445 | −5.3665 | −2.5185 | −1.8955 | −2.5445 | −2.0595 |
| ERP70 | −0.593 | −0.545 | −0.651 | 0.255 | −0.611 | 0.149 | −0.187 | −0.052 | −0.152 | −2.258 | −0.321 | 1.533 | 1.075 | 0.488 | 0.404 |
| ESR1 | 0.0845 | 0.4435 | 0.6515 | 0.2815 | 0.8525 | 0.5205 | 0.0465 | 0.8435 | 0.2015 | 0.8825 | 2.1415 | −0.0185 | 0.2045 | 0.2585 | 0.6675 |
| ETFA | 0.8525 | 0.1225 | 0.6295 | −0.0355 | −0.2055 | −0.1065 | −0.1035 | 0.1355 | −0.4735 | −0.8085 | 0.0925 | −0.0985 | −0.1745 | 1.3165 | −0.5885 |
| ETV8 | 0.3005 | 0.2495 | 0.8085 | 0.9515 | 1.7405 | 0.8465 | 1.5535 | 0.7345 | 1.1665 | 0.2855 | 1.0645 | 1.0905 | 0.2765 | 1.6565 | 0.7165 |
| EVER1 | −0.0405 | −0.5975 | 0.2095 | 0.0035 | −0.2235 | 0.0185 | −0.0575 | 0.4335 | 0.1745 | 0.4945 | 0.5295 | −0.5545 | −0.3845 | 0.1725 | 0.4685 |
| EXO1 | 0.7615 | 0.3615 | −0.2415 | 0.8075 | 0.6245 | −0.1905 | −0.0815 | 0.5515 | 0.3405 | 0.7515 | 0.2835 | −0.1695 | 0.9385 | 0.8425 | 0.7226 |
| EXOSC9 | 0.739 | 1.236 | 0.438 | 0.984 | 1.612 | 0.241 | 2.412 | 0.916 | 1.493 | 1.753 | 1.415 | 0.592 | 0.499 | 0.826 | 1.57 |
| EXT2 | 0.346 | −0.023 | −0.083 | −0.144 | −2.313 | −0.107 | −0.455 | −0.982 | −1.02 | −0.282 | 0.063 | 0.284 | −0.903 | −0.6 | −0.736 |
| EZH2 | −0.1765 | −0.0285 | 0.0455 | 0.0065 | −0.0795 | −0.2735 | 0.2785 | 0.0595 | −0.0465 | −0.5335 | 0.0285 | 0.4425 | 0.0875 | −0.0045 | 0.2675 |
| FI1R | 1.745 | −0.516 | 0.101 | −0.537 | −2.938 | −0.01 | 0.351 | −0.764 | 0.808 | −1.254 | −0.047 | 0.01 | 2.537 | −0.114 | −1.929 |
| F9 | 1.766 | 3.194 | −0.748 | 1.841 | 5.097 | 4.824 | 2.868 | 1.666 | 0.536 | 3.972 | 7.007 | −0.83 | 3.775 | 1.449 | 0.827 |
| FA2H | 0.409 | −0.262 | 0.211 | 0.838 | 0.327 | 0.101 | −0.097 | 0.237 | 0.313 | −0.223 | −0.198 | −0.439 | 0.007 | −0.020 | 0.431 |
| FABP7 | −0.429 | −0.254 | 0.388 | 0.524 | 0.506 | 0.089 | 0.885 | 0.356 | 0.347 | −0.281 | −0.632 | 0.66 | −0.18 | 0.808 | −0.822 |
| FADD | 0.783 | 0.096 | 0.904 | 0.777 | 1.19 | 1.147 | 0.288 | −0.275 | −0.77 | 0.233 | 0.778 | −0.282 | −0.881 | −0.211 | −0.094 |
| FAHD1 | −0.339 | 0.301 | −1.616 | −0.002 | 1.051 | 0.324 | 1.543 | 0.828 | 0.535 | 0.59 | −0.18 | 0.954 | −0.104 | 0.294 | −0.344 |
| FAHD2A | 0.968 | 0.546 | −0.508 | −0.202 | 0.18 | 0.464 | −0.172 | −0.354 | −0.557 | −0.308 | −0.187 | 0.71 | −0.227 | −0.239 | −0.313 |
| FAM38A | 1.3095 | 1.5695 | −0.8095 | −1.1405 | −1.1705 | −0.8485 | 0.0855 | −0.0885 | 0.5715 | −0.5805 | 0.9685 | 0.0825 | 0.5645 | −0.7535 | 0.8035 |
| FAM3A | 0.4745 | 0.8145 | −1.4835 | 0.9955 | 0.4535 | 0.3045 | 2.2125 | 1.4445 | 0.0205 | 0.8265 | −0.4175 | 1.4305 | 1.5955 | 0.8685 | 0.5075 |
| FAM3B | −1.051 | −0.812 | −0.542 | −0.539 | −1.076 | −0.936 | −0.834 | −0.208 | 0.225 | −0.888 | −0.04 | −0.827 | 0.388 | −0.052 | 0.088 |
| FAM3C | 0.072 | 0.657 | 1.21 | 0.346 | 0.085 | 1.31 | 1.569 | 0.533 | 0.121 | 0.243 | 0.478 | 1.7 | 0.984 | 0.851 | 1.331 |
| FAM46C | 1.273 | 0.182 | 1.558 | 1.868 | 0.782 | 1.19 | 1.015 | 1.075 | 1.522 | −0.404 | 0.71 | 0.374 | 0.733 | 2.079 | 0.58 |
| FAM49B | 0.876 | 0.616 | 0.868 | 1.467 | 0.119 | 1.307 | 0.237 | 1.367 | 1.767 | 0.774 | 0.742 | 1.759 | 1.437 | 0.957 | 1.657 |
| FAM54A | 0.305 | −0.188 | 0.914 | 0.064 | 0.543 | 0.688 | 1.403 | 1.723 | 0.791 | 0.244 | 1.783 | 1.25 | 0.748 | 1.034 | 1.504 |
| FANCA | 0.2485 | 0.1685 | 0.3105 | 0.4435 | 1.1105 | 1.6875 | 0.5735 | 0.4005 | 0.3225 | 0.7105 | 1.1705 | −0.7555 | 0.4845 | −0.3945 | 0.5635 |
| FANCE | −2.6345 | −3.0205 | −1.3835 | −1.5475 | −2.5825 | −0.9975 | −2.6135 | −0.6605 | −1.2985 | −2.7065 | −1.8425 | −1.7295 | −0.8225 | −2.5155 | −2.5365 |
| FBL | −0.286 | −0.318 | −0.47 | −0.335 | −0.513 | −0.251 | −0.824 | −0.352 | −0.652 | −0.148 | 0.013 | −0.685 | −0.145 | 0.077 | 0.375 |
| FBP1 | −0.281 | −0.809 | −1.176 | −0.42 | −0.144 | −0.331 | 0.179 | 0.07 | 0.096 | 0.23 | −0.467 | 0.828 | 0.307 | 0.218 | 0.331 |
| FBS1 | −0.0925 | −0.9165 | −0.6665 | 0.0225 | −0.0865 | 0.7205 | 0.4135 | −0.1995 | −0.0435 | −0.0845 | 0.3585 | −0.3785 | −0.6475 | −0.1885 | 0.0645 |
| FBXL20 | 0.544 | −0.878 | 0.651 | −0.531 | −0.346 | 0.201 | −1.213 | −0.648 | 0.044 | 0.602 | −0.534 | −1.188 | −0.824 | 0.751 | −0.09 |
| FBXO25 | 0.317 | −0.473 | −0.38 | 0.317 | −0.198 | 0.524 | 0.264 | −0.036 | 0.171 | −0.188 | −0.637 | 1.065 | 0.612 | −0.146 | −0.334 |
| FDXR | −0.825 | −0.768 | −1.088 | −0.142 | −0.316 | 0.135 | −0.669 | −0.272 | 0.098 | −0.298 | −0.667 | −0.448 | 1.166 | −0.094 | −0.578 |
| FEM1B | −0.712 | 1.11 | −1.821 | 1.656 | 1.375 | 0.38 | 1.930 | 0.41 | 0.578 | −1.111 | −1.164 | 2.599 | 0.124 | −0.1 | 0.835 |
| FGF12 | 0.1205 | −0.4925 | −0.7655 | 0.3275 | 0.1605 | 0.1885 | 0.1585 | −0.3715 | −0.7065 | −0.7535 | −0.8025 | −0.8835 | −0.2665 | −0.6985 | −0.8565 |
| FGF13 | 0.04 | −0.321 | −0.486 | 0.7 | −0.877 | 0.11 | −0.171 | 0.52 | −0.133 | −0.425 | −0.33 | 0.238 | 0.895 | 1.797 | 0.529 |
| FHOD1 | 0.9455 | −0.7715 | −0.8625 | −0.7895 | −0.5035 | −0.1865 | −0.0825 | −0.0145 | −0.2005 | −0.4135 | 0.8075 | −0.5115 | −0.4785 | 0.2815 | −0.1355 |
| FKBP11 | −0.8915 | −2.1705 | −0.5975 | 0.6375 | −0.9185 | −1.0175 | −0.6025 | −0.2195 | −1.3135 | −1.3135 | −1.0995 | 0.0205 | 0.2285 | −0.6625 | −1.3695 |
| FLJ10094 | 0.521 | 1.212 | 0.229 | 1.14 | −0.541 | 0.601 | 0.333 | −0.177 | 0.158 | 0.154 | 0.958 | −0.26 | 0.116 | −0.370 | 0.202 |
| FLJ10115 | 0.1625 | 1.0625 | 0.4265 | −0.4085 | 0.8245 | 0.8555 | 1.3335 | 0.6185 | −0.7625 | 0.4455 | 0.6785 | 0.6115 | −0.0635 | 0.1275 | 0.6215 |
| FLJ10159 | −0.123 | −0.347 | −0.904 | −0.444 | 0.944 | −0.761 | −0.517 | −0.54 | 0.24 | 0.19 | 0.178 | −1.214 | −0.3 | −0.384 | 0.405 |
| FLJ10204 | 0 | 1.018 | 1.265 | 0.479 | 0.338 | 0.199 | 0.372 | 0.858 | −0.021 | 0.113 | 0.836 | 1.016 | 1.45 | 1.092 | 1.051 |
| FLJ10275 | 0.3295 | −2.4285 | −0.5725 | −0.4965 | 1.3465 | 1.6675 | −1.1805 | −1.3265 | −0.6535 | −1.4685 | −0.8205 | 3.1395 | −0.6525 | 0.6105 | −1.1035 |
| FLJ10292 | −0.3835 | −0.1835 | −0.5325 | −0.4005 | 0.0685 | −0.0675 | 0.3585 | 0.5325 | 0.4585 | −0.0165 | 0.1265 | 0.6125 | 0.5655 | 0.7795 | 1.4615 |
| FLJ10324 | −0.3185 | −0.6325 | 0.4795 | 1.1045 | 1.0085 | 0.5535 | 1.2615 | 1.3535 | 1.5145 | 0.5725 | 0.7945 | 1.3125 | 1.1395 | 1.3185 | 0.9145 |
| FLJ10700 | 0.432 | −0.703 | 0.041 | 0.068 | −0.687 | 0.348 | 0.064 | −0.398 | 0.669 | 0.18 | 0.781 | 0.084 | −0.497 | 0.698 | 0.17 |
| FLJ10706 | 1.422 | −0.466 | 1.843 | 0.639 | −0.264 | 0.462 | 2.586 | 3.676 | 1.258 | 1.047 | 1.952 | 2.624 | 2.003 | 1.339 | −1.376 |
| FLJ10671 | −0.59 | 0.838 | −0.127 | −0.5 | −0.063 | 0.133 | −0.745 | −0.758 | −0.355 | −0.812 | −0.001 | −1.602 | −0.294 | 0.441 | −1.273 |
| FLJ10901 | −0.5705 | −0.7915 | −1.6165 | −1.1555 | −1.8275 | −1.3585 | −1.1825 | −0.9825 | −0.8165 | −1.2725 | −0.9325 | −0.8865 | −1.7295 | −1.2725 | −1.2725 |
| FLJ10918 | 0.610 | 0.30 | 1.062 | 1.881 | −0.521 | 0.601 | 0.642 | −0.062 | −0.46 | −0.17 | 0.571 | 1.009 | 0.821 | 2.552 | 0.448 |
| FLJ10980 | −0.8076 | 0.6635 | −0.1425 | 0.0035 | −0.8485 | −0.0725 | −0.0475 | 0.6216 | 0.4515 | 0.0045 | 0.0355 | 0.2125 | 0.1305 | −0.0115 | 1.2085 |
| FLJ11017 | 0.8595 | −0.6145 | 0.3945 | −0.7785 | −0.7315 | −0.1405 | −0.5195 | 0.4185 | −0.6885 | −1.0885 | −0.8915 | 0.5475 | 1.3785 | 1.3365 | 0.1725 |
| FLJ11088 | −0.8825 | 1.0025 | −0.3475 | 0.8575 | 0.7015 | 0.7235 | 0.3895 | −0.1325 | 0.2065 | 0.0155 | 0.9085 | 1.2415 | 0.7195 | −0.0645 | 0.4965 |
| FLJ11151 | 0.0175 | −0.2915 | −1.3285 | −0.3655 | 0.0555 | −0.1675 | −0.2665 | −0.4605 | −0.5065 | −0.5865 | −0.7655 | −0.4316 | −0.9055 | −0.1565 | −0.6355 |
| FLJ11267 | −0.5845 | −0.3515 | −0.9185 | −0.9345 | −0.4885 | −0.0605 | −0.9695 | −0.6415 | −0.2415 | −0.5315 | −0.0525 | 0.0235 | −0.0715 | −0.7215 | −1.0565 |
| FLJ11280 | 0.2985 | 0.3425 | −0.4365 | 0.1465 | −0.0945 | 0.6465 | 0.4525 | 0.1325 | −0.1985 | 0.3325 | 0.8255 | −0.4935 | −0.8575 | −0.0675 | 0.5145 |
| FLJ11508 | 0.1715 | −0.4125 | −0.5705 | 0.0325 | −0.4065 | 1.4975 | 0.8825 | 0.4995 | −0.0515 | −0.7615 | 0.1485 | 0.0345 | −0.1095 | −0.6235 | 1.4755 |
| FLJ12270 | −1.6555 | −1.8015 | −2.4205 | −1.3705 | 1.0415 | −2.0105 | 1.2245 | −0.8575 | −0.0235 | −1.0845 | −1.1055 | 1.1345 | −0.7185 | 0.1075 | −0.9875 |
| FLJ12844 | 0.1095 | −0.4455 | −0.4425 | 0.0495 | −0.3805 | 0.7835 | 0.4385 | 0.9875 | −0.0775 | 0.0795 | 0.4245 | 1.3855 | 0.6085 | 0.6825 | 0.1745 |
| FLJ12650 | −0.3215 | 0.2755 | 0.4095 | 0.7815 | 0.1845 | 0.0085 | 0.1435 | 0.2705 | 0.4225 | 0.3405 | 0.3705 | 0.0275 | −0.3545 | 0.1505 | 0.6165 |
| FLJ12684 | 0.7215 | −0.4125 | 0.4015 | 0.7365 | 0.5815 | 0.6055 | 0.0205 | −0.2725 | −0.5885 | 0.5395 | 0.9705 | −0.0785 | −0.1855 | −0.0525 | −0.1845 |
| FLJ12735 | 0.357 | −0.517 | −1.397 | −1.503 | −1.307 | −0.307 | 0.137 | −0.49 | 0.014 | −0.6 | −0.184 | −0.622 | −0.632 | −0.34 | 0.177 |
| FLJ12750 | −1.922 | −3.098 | −2.805 | −0.747 | −3.628 | −1.54 | −2.868 | −2.295 | −1.904 | −3.424 | −4.381 | −1.192 | −1.459 | −2.019 | −1.447 |
| FLJ12895 | −0.8275 | −1.1245 | −0.4545 | −0.8095 | 0.0545 | 0.0005 | 0.0895 | 0.3125 | 0.2916 | −0.4705 | −0.2995 | −0.2695 | −0.3575 | −0.3655 | 0.6355 |
| FLJ13710 | 1.6495 | −0.3495 | 0.7675 | 0.6985 | 0.4555 | 0.9545 | 0.7885 | 0.3925 | 0.0245 | −0.4945 | 0.9085 | 0.4315 | 0.3895 | 0.5665 | 0.5335 |
| FLJ13855 | −0.071 | 0.139 | 0.056 | −0.587 | 0.815 | −0.587 | 0.353 | 0.54 | 0.193 | −0.384 | 0.103 | 0.46 | 0.543 | 0.628 | 0.647 |
| FLJ13912 | 1.68 | 0.596 | 0.21 | 0.036 | 0.548 | 0.42 | −0.644 | 0.465 | 0.796 | 1.547 | 2.374 | 0.074 | 0.082 | 0.402 | 0.55 |
| FLJ14124 | 0.882 | 0.228 | 0.294 | 0.602 | −0.019 | −0.161 | −0.141 | 0.482 | 0.277 | −0.199 | 0.829 | 0.928 | −0.031 | 0.538 | 0.346 |
| FLJ14627 | −0.0975 | 0.1195 | −0.1125 | 0.7675 | −0.1435 | 0.0985 | −0.2055 | 0.0285 | −0.0515 | 0.1255 | 0.2445 | −0.2775 | 1.1585 | 0.1155 | 0.0135 |
| FLJ14666 | 0.409 | 0.878 | 0.434 | 0.527 | 0.057 | −0.027 | −0.208 | −0.229 | −0.864 | −0.035 | 0.168 | 0.536 | 0.01 | 0.793 | 0.685 |
| FLJ14904 | 1.741 | 1.021 | −1.255 | −1.262 | −2.844 | 0.015 | −1.387 | −1.3 | 0.305 | −2.095 | −1.143 | −1.846 | 0.074 | −1.314 | −0.871 |
| FLJ20035 | 0.524 | 0.239 | 0.592 | −0.215 | 0.924 | 2.095 | 0.008 | −0.272 | −0.006 | −0.229 | −0.133 | 0.065 | −0.206 | 1.162 | −0.287 |
| FLJ20152 | 0.38 | 0.626 | 0.126 | 0.579 | 1.105 | 0.158 | −0.458 | −0.421 | 0.158 | −0.127 | 0.417 | −0.931 | 0.375 | 0.888 | 2.13 |
| FLJ20171 | −0.073 | −0.119 | 0.457 | 0.416 | −0.500 | −0.385 | −0.403 | 0.026 | −2.541 | −0.893 | −1.06 | 0.213 | −1.29 | 0.11 | −1.331 |
| FLJ20244 | −1.694 | −0.606 | −2.204 | −1.51 | 0.309 | 0.335 | 0.138 | −1.088 | −1.542 | 0.552 | 0.609 | −2.365 | −1.777 | −1.774 | 0.119 |
| FLJ20273 | 0.193 | 0.074 | −0.503 | −0.168 | −0.547 | −0.374 | −0.175 | −0.194 | −0.796 | −0.874 | −0.354 | 0.235 | −0.112 | 0.174 | −0.359 |
| FLJ20315 | 0.648 | 0.35 | 0.864 | 0.814 | 0.339 | 0.902 | 0.51 | −0.035 | −0.002 | −0.198 | 0.753 | 1.152 | 1.2 | 0.52 | 0.818 |
| FLJ20323 | 0.2805 | −0.0975 | 0.5105 | 0.3545 | 1.2485 | 1.3065 | 0.4165 | −0.4845 | −0.3245 | −0.0795 | 0.6085 | 0.4745 | −0.4475 | 0.5525 | −0.0035 |
| FLJ20518 | −1.667 | −1.125 | 0.145 | 0.516 | −0.188 | −0.011 | −0.404 | 0.731 | −0.372 | −0.366 | −0.126 | 0.054 | 0.034 | 0.175 | 0.437 |
| FLJ20530 | 0.079 | 0.878 | 0.663 | 0.282 | 0.342 | 0.238 | −0.112 | 0.489 | −0.118 | −0.139 | 0.207 | 0.635 | 0.732 | 0.043 | 0.409 |
| FLJ20696 | −0.1455 | 0.0965 | 0.5315 | −0.0475 | 1.2285 | 0.7925 | 0.8085 | −0.0355 | −0.7955 | −0.5155 | 0.0525 | 0.8515 | −0.2355 | −0.6975 | 0.3635 |
| FLJ20718 | 0.745 | 1.583 | 0.839 | −0.126 | 0.734 | 0.132 | 0.606 | −0.831 | −0.171 | −0.434 | 0.225 | −0.192 | −0.346 | 0.007 | −0.617 |
| FLJ20772 | −1.8785 | −0.4805 | −0.8125 | −0.7285 | −0.5155 | −0.3165 | 0.4965 | −0.4955 | −0.9125 | −0.4105 | 0.2875 | −1.7975 | −1.0185 | −0.9765 | −0.9125 |
| FLJ20989 | −0.3335 | −0.2205 | −0.0265 | −0.4005 | −0.3765 | 0.0265 | −0.4035 | −1.0495 | −0.3745 | 0.1705 | −0.7305 | −1.2975 | −0.6145 | −0.5195 | −0.0335 |
| FLJ21019 | −0.1465 | −0.7515 | −0.7165 | −1.1435 | −0.3805 | 0.3585 | −0.7835 | 0.0515 | 0.0485 | −0.3915 | −0.3685 | −0.8805 | −1.5385 | −0.0215 | 1.3855 |
| FLJ21062 | −2.4065 | −0.2005 | 0.0975 | 0.2905 | −0.2565 | −1.0805 | 0.4015 | −0.9135 | −2.1155 | 0.0815 | 1.2615 | 0.5355 | −0.1175 | 0.9955 | 0.3385 |
| FLJ21159 | −1.7105 | −1.6825 | −1.0255 | −1.1945 | −0.5075 | −0.0685 | 0.1115 | −0.0085 | 0.0135 | −1.3805 | −1.1255 | −2.1065 | −0.9245 | −0.7855 | 0.1695 |
| FLJ21616 | −1.994 | −1.495 | −0.144 | −1.29 | −1.892 | −0.615 | −1.53 | 0.849 | −0.893 | −0.253 | 0.919 | −2.107 | −0.345 | −1.214 | −0.827 |
| FLJ21827 | −0.0485 | −0.5085 | −1.1155 | −0.0005 | 0.1625 | −0.0855 | 0.6835 | −0.2165 | −1.4635 | −0.1775 | −0.5625 | −0.1565 | −0.8135 | 0.5615 | −1.0675 |
| FLJ21963 | 1.388 | −0.565 | −0.137 | −0.889 | 0.146 | 0.342 | −0.254 | −0.148 | −0.711 | 0.289 | 0.241 | −0.956 | −0.205 | 1.075 | 0.154 |
| FLJ22104 | −0.1205 | −0.0385 | 1.0425 | 0.3905 | −2.2195 | 0.4455 | −0.8475 | −1.5445 | 0.6895 | −0.3675 | −2.2375 | −0.1135 | 0.4165 | 0.2585 | 0.5125 |
| FLJ22222 | −0.465 | −0.963 | −0.919 | −0.802 | −0.933 | −0.608 | −1.235 | −0.647 | 0.355 | 0.072 | −0.307 | −0.238 | −0.645 | −0.406 | −0.109 |
| FLJ22573 | 0.2825 | −1.9335 | −0.9285 | −1.7485 | −1.3285 | −0.1165 | −1.1775 | −0.3855 | −0.0995 | −0.2315 | −0.8305 | 0.1955 | −0.1865 | −0.3075 | −1.0615 |
| FLJ22794 | 0.659 | 1.032 | 0.183 | 0.696 | 1.798 | 0.817 | 1.322 | 0.023 | 0.238 | −0.058 | 0.77 | −0.059 | 0.366 | 1.079 | 0.178 |
| FLJ23188 | −0.3725 | 1.0075 | −0.9055 | −0.5375 | 0.7805 | 0.0895 | 0.5005 | 0.0385 | 0.2185 | 0.8316 | 0.4355 | −0.1575 | 2.3715 | 2.1725 | −0.1885 |
| FLJ23441 | −0.611 | −0.924 | −0.598 | −2.472 | −0.177 | −0.529 | −0.461 | −0.979 | −1.196 | −0.825 | −0.991 | −0.284 | −1.205 | 1.767 | 0.147 |
| FLJ25471 | −0.257 | −0.204 | 0.29 | 0.035 | 0.665 | 0.24 | 0.313 | 0 | −0.407 | −0.128 | 0.637 | −0.403 | 0.431 | −0.017 | −0.044 |
| FLJ31204 | −0.9255 | −0.8055 | −1.0185 | −0.9635 | −0.9315 | −1.0935 | −0.3745 | −0.0015 | 0.0975 | −0.1695 | −1.6585 | 0.0205 | 2.8855 | −0.3085 | 0.0995 |
| FLJ31795 | −0.4075 | −0.8405 | 0.3945 | 0.1485 | −0.5175 | 0.0565 | −0.2005 | 0.2095 | 0.9985 | 0.0015 | 0.2835 | 0.0085 | −0.2485 | −0.1895 | 0.0895 |
| FLJ32942 | −1.7155 | −3.3555 | −2.1885 | 1.3285 | 0.4845 | 0.2315 | −2.5085 | −1.8495 | −0.9995 | −2.2385 | −3.2975 | −0.8205 | −1.2505 | −1.0085 | 0.0835 |
| FLJ37970 | 2.678 | 2.607 | 0.43 | 1.934 | 1.758 | 1.696 | 3.653 | 2.145 | 2.346 | 2.475 | 0.605 | 1.715 | 0.85 | 0.892 | −0.219 |
| FLJ39370 | 1.0495 | −0.5255 | −0.3005 | −1.1575 | −0.4235 | −0.2625 | 0.5455 | −0.1345 | −1.0015 | 0.3615 | 0.2225 | −0.8276 | −0.2535 | 0.3655 | 0.2735 |
| FLJ90588 | 1.4335 | 1.4755 | 0.7925 | 1.4295 | −0.6655 | 0.0075 | 0.1955 | −0.4415 | −0.0665 | 1.5525 | 1.5805 | 2.2005 | 1.3525 | 2.7405 | −0.5305 |
| FN3KRP | −6.357 | −5.56 | −5.652 | −5.915 | −8.405 | −2.126 | −3.892 | −5.216 | −5.585 | −4.091 | −5.001 | −5.373 | −5.628 | −5.848 | −6.446 |
| FOLR1 | 4.0205 | 3.3305 | 3.0805 | 2.7405 | 3.8305 | 2.0175 | 3.4505 | 2.3705 | 1.7205 | 3.9705 | 3.7105 | 2.4905 | 1.4695 | 3.4965 | 0.8225 |
| FOXA1 | −1.3905 | −0.7475 | −1.3155 | −0.9925 | −0.2685 | −0.2745 | −0.8465 | −0.6005 | −0.2535 | −0.0285 | −1.1195 | −1.8875 | −0.9775 | −0.8915 | −0.8225 |
| FOXC1 | −0.01 | 1.644 | 1.195 | 0.964 | 2.133 | 0.928 | 0.959 | 0.286 | 0.735 | 1.17 | 0.551 | 0.647 | 0.473 | 1.657 | 1.202 |
| FOXP1 | 0.1165 | −0.0905 | −0.0735 | −0.2675 | −0.7015 | 0.1455 | −0.2515 | −0.2695 | 0.9595 | 0.1395 | 0.2345 | −0.5765 | −0.8595 | −0.5095 | −0.0055 |
| FREQ | 0.5545 | −0.0355 | 0.5665 | −0.3575 | −0.0205 | 0.5645 | −0.4135 | −0.2425 | −1.0265 | −0.2065 | −0.2515 | 0.3845 | −0.1305 | 0.1195 | −0.0595 |
| FTO | −0.8115 | −0.8135 | −0.4155 | 0.4875 | −1.3615 | −0.3535 | −0.8135 | −0.9855 | −1.0585 | −0.8665 | −0.1885 | −0.4305 | −0.5545 | −1.7125 | −0.5975 |
| FUBP1 | −0.8675 | −2.1045 | −0.9215 | −0.7695 | −0.8165 | −0.1655 | −0.5385 | −0.5745 | −0.8005 | −0.6545 | −1.7275 | −1.7875 | −1.3845 | −0.9345 | −1.5225 |
| FUCA1 | 1.238 | −1.057 | −0.697 | −0.409 | −0.717 | 1.114 | 2.181 | 1.468 | 1.539 | 1.745 | 1.704 | 0.359 | 0.628 | −0.934 | −0.135 |
| FUT8 | −1.0195 | −1.7555 | −1.1795 | −1.3455 | −0.1115 | −0.5225 | 0.0485 | 0.0855 | −0.6475 | −1.9035 | −1.4155 | 0.3595 | −0.5185 | −0.8625 | −1.2716 |
| FXYD6 | −1.043 | −1.69 | −1.757 | 0.584 | 1.877 | 0.297 | 0.317 | −0.788 | −0.039 | −0.596 | 0.353 | 2.457 | −0.494 | 0.407 | 0.245 |
| FYCO1 | −0.018 | 0.721 | 2.166 | 1.242 | −1.38 | 0.967 | 2.647 | 1.77 | 1.795 | 2.135 | 2.276 | 1.015 | 0.322 | 0.355 | 2.568 |
| FZD10 | 1.0395 | −0.4385 | 1.2775 | 2.1525 | 3.9085 | 1.5755 | 2.9535 | 0.2125 | 0.3205 | 0.7595 | 0.3175 | 2.7855 | 0.6405 | 0.5095 | 0.1105 |
| FZD7 | 2.2965 | 2.2275 | 0.4445 | 0.3575 | 1.9185 | 0.5985 | −1.3705 | −0.7355 | −0.0065 | −0.1505 | −0.6835 | 0.7185 | 0.6235 | 1.9665 | 1.5555 |
| FZD9 | 2.7065 | 2.1776 | −0.1385 | 0.5085 | 1.0275 | 0.6205 | −1.3905 | −1.0435 | −0.5805 | 1.0215 | −0.5585 | −0.3665 | 0.7575 | 1.3675 | 1.0125 |
| G1P2 | −0.2015 | 0.2985 | 0.9165 | 1.1785 | −0.0215 | −0.0495 | −0.1715 | −0.0765 | 0.4536 | 0.1295 | 0.1795 | 0.0955 | 1.4955 | 1.4565 | 0.7005 |
| G1P3 | −0.363 | 0.923 | −0.139 | −0.003 | −0.61 | 0.45 | 1.391 | 0.292 | 0.252 | 0.198 | −0.217 | 0.829 | 0.817 | −0.412 | 1.046 |
| GA17 | −0.0016 | −0.5685 | 1.6165 | 1.6845 | 1.0645 | 1.3625 | 0.9595 | 1.1075 | 0.4555 | 0.7625 | 0.8375 | 1.6885 | 1.8745 | 0.4155 | 2.6335 |
| GABARAP | 0.4015 | −0.4345 | −0.2115 | 0.0045 | −0.4835 | −0.8975 | −1.1965 | −1.1835 | −0.8195 | −0.8995 | −0.6975 | −0.8025 | 0.2905 | 0.6825 | −1.2885 |
| GAJ | 2.3175 | −1.3495 | 1.2445 | 0.4225 | −0.9125 | 0.5745 | 0.2555 | 0.0415 | 1.7035 | −1.1325 | 1.5185 | 3.9225 | 1.7105 | 3.4775 | −0.7325 |
| GALE | 1.053 | 0.012 | −0.303 | −0.129 | 0.891 | 0.07 | 0.368 | 0.047 | 0.182 | 0.364 | −0.004 | 0.222 | 0.458 | 1.803 | 0.387 |
| GALNT14 | −1.8055 | −1.3105 | −1.3825 | −0.4635 | −1.8675 | −1.8635 | −2.5215 | −0.3445 | −1.0985 | −2.0435 | −0.8995 | 0.3225 | −0.8955 | −0.3745 | −0.1855 |
| GALNT2 | −2.6555 | −1.7555 | −1.0975 | −1.3765 | −0.1805 | −0.7615 | −0.8676 | −1.1925 | −2.4185 | −1.8005 | −2.2235 | −1.3395 | −1.1605 | −2.1165 | −1.5945 |
| GALNT7 | 0.714 | 0.351 | 1.225 | −0.381 | −0.844 | 0.779 | −0.005 | 0.97 | 0.146 | 0.005 | 0.108 | −0.91 | 0.168 | −0.091 | 0.478 |
| GAMT | −0.583 | 1.049 | 1.383 | 0.378 | 0.827 | 0.191 | 0.081 | −0.043 | 0.491 | 0.03 | 0.535 | 0.594 | 0.158 | −0.091 | 0.395 |
| GAPD | 0.471 | 0.132 | 0.731 | 0.48 | 1.039 | 0.983 | 0.359 | −0.035 | 0.279 | 0.322 | 0.845 | −0.47 | 0.899 | 1.828 | 0.651 |
| GARS | −1.904 | −2.688 | −2.875 | −1.827 | −1.395 | −1.535 | −3.675 | −3.58 | −2.401 | −2.981 | −3.232 | −1.914 | −2.759 | −2.727 | −0.862 |
| GART | −0.8815 | −2.7455 | −0.5605 | −0.9275 | −2.8345 | 0.7155 | −1.3955 | −1.2355 | −0.2585 | −2.5685 | −0.3745 | −0.3645 | −0.0005 | −1.3095 | 2.2585 |
| GATA3 | 1.0495 | −0.6685 | −0.3955 | −0.5405 | 1.1185 | −0.0685 | 0.2355 | 0.8225 | −0.5815 | −0.2075 | 0.3895 | 1.0115 | −0.5335 | 1.0565 | −0.0205 |
| GATM | 0.487 | −0.827 | 0.282 | −1.185 | 1.765 | 1.406 | −1.343 | −1.056 | −0.839 | −0.675 | 0.257 | −1.156 | −0.587 | −0.758 | −1.433 |
| GBA | −0.378 | −0.481 | −0.069 | −0.319 | 1.493 | 1.165 | 0.181 | 0.228 | −0.044 | 0.148 | 0.618 | 0.311 | 0.178 | 0.746 | 1.877 |
| GDAP1 | −1.124 | −0.015 | 0.14 | −0.405 | 0.031 | 0.107 | −0.367 | −0.032 | −0.228 | −0.193 | 0.859 | −0.324 | −0.432 | −0.344 | −0.417 |
| GDI2 | −0.0375 | −0.3415 | −0.5065 | −1.8935 | −2.6995 | −0.4355 | −0.3195 | −1.3705 | −0.6455 | −0.0455 | −0.1535 | 0.2505 | −0.1145 | −0.8985 | 0.8805 |
| GGA1 | −0.2915 | −0.2045 | −0.8505 | −0.2745 | −0.4005 | −1.0735 | −1.1555 | −0.7985 | −0.3435 | −0.8365 | −0.7595 | −1.3035 | −0.8115 | −0.5565 | −0.8595 |
| GKAP1 | 0.3925 | −0.0005 | −0.9025 | −0.5145 | −0.4285 | 0.6585 | 0.1775 | 0.0885 | 0.6635 | 0.1065 | 0.2865 | −0.7916 | −0.0615 | −0.3585 | 0.8815 |
| GLCCI1 | −0.7855 | −0.2115 | −0.7945 | −0.5735 | −1.4685 | 0.4795 | −0.2005 | −0.0845 | 0.0255 | 0.8755 | 0.0085 | −1.4315 | −1.0435 | −1.4975 | −0.4205 |
| GLG1 | 0.979 | −1.119 | −0.022 | 0.834 | 1.829 | 1.289 | 2.634 | 0.774 | 0.003 | 0.788 | 1.124 | 1.312 | 0.34 | 1.041 | 0.905 |
| GLTSCR2 | 0.018 | −1.873 | −0.619 | −1.028 | −1.831 | 0.195 | −0.849 | 0.318 | −0.022 | −0.452 | −0.176 | 0.372 | 0.181 | −0.721 | 0.335 |
| GNB4 | 0.459 | 0.102 | 0.135 | −0.236 | 1.083 | 0.583 | 0.049 | 0.725 | 0.424 | 0.175 | 0.299 | −0.426 | −0.456 | −1.149 | −0.648 |
| GOLPH2 | −0.3675 | 0.1185 | −0.6155 | −0.0265 | −0.3475 | −0.1385 | 1.8755 | 0.6985 | 0.4085 | 0.7875 | 1.3865 | 0.2955 | 0.1845 | 0.5335 | 0.5695 |
| GPATC1 | −0.5535 | 0.2725 | −1.7705 | −0.9055 | 0.1495 | 1.1755 | 2.1585 | 1.3125 | 0.4825 | 0.8355 | 0.2855 | 0.3315 | 0.2785 | 0.5145 | 0.8305 |
| GPC2 | −0.8425 | −1.1415 | −2.7576 | −0.2535 | −2.8995 | −1.1925 | −2.3545 | −1.2915 | −0.2925 | −1.6685 | −1.6895 | −1.5795 | 0.5285 | −1.8675 | −1.7705 |
| GPR125 | 1.142 | 0.157 | 0.217 | 1.736 | 0.325 | 0.312 | 0.175 | 1.081 | 0.613 | 0.831 | 0.32 | 1.521 | 1.071 | 0.55 | −0.326 |
| GPR160 | −0.4745 | −2.2815 | −1.0285 | −0.0135 | −1.3515 | −0.5565 | −1.8745 | −1.6215 | −2.2195 | −1.6235 | −2.2455 | −0.4285 | −0.8055 | −1.5285 | −1.7385 |
| GPR56 | −0.5385 | −0.2295 | −1.1235 | −0.2625 | −0.0725 | −0.3515 | 0.0745 | −0.0615 | 0.0785 | 0.7295 | 0.1215 | 0.4035 | −0.3145 | −0.4335 | 0.8495 |
| GPRC5C | 1.8375 | −0.1085 | 1.0645 | 0.4095 | 2.4875 | 1.6935 | 2.0915 | 1.2545 | 0.2535 | 0.5685 | 1.5185 | 1.5025 | −0.2315 | 1.1635 | 1.8135 |
| GPSM1 | −0.714 | 0.417 | 0.585 | −0.175 | 0.351 | −1.108 | 0.594 | 0.242 | −0.258 | −0.991 | 0.052 | −2.148 | −0.808 | 0.563 | −0.704 |
| GPSM2 | 1.831 | 1.648 | 1.783 | 1.041 | 1.69 | 1.698 | 0.473 | 0.645 | −0.299 | 0.346 | 2.115 | 0.673 | 0.353 | −0.594 | 1.213 |
| GPT | 2.4425 | −0.9205 | 0.3455 | −0.1465 | 0.7325 | 0.2335 | 1.2035 | 0.4045 | 1.8135 | 0.8555 | 1.3975 | −1.3295 | 0.7365 | 1.0075 | 1.1655 |
| GPT2 | 1.142 | 3.177 | −1.37 | 3.835 | 3.272 | 1.118 | 2.407 | 3.382 | 0.555 | 0.329 | 0.854 | 0.57 | 1.454 | 1.182 | 0.888 |
| GPX7 | −0.26 | −0.12 | −0.108 | −0.333 | −0.401 | −0.32 | −0.152 | 0.106 | 0.108 | 0.39 | 0.164 | 0.02 | 0.08 | −0.343 | −0.306 |
| GRB14 | −1.6825 | −2.0235 | −2.1025 | −1.3975 | −0.9605 | −0.0515 | −0.2145 | −1.2025 | 0.5705 | 0.0065 | −1.6655 | −1.6435 | −0.7705 | −0.8525 | −0.9445 |
| GRB7 | −0.6695 | −0.0945 | −0.5145 | −0.6725 | −0.0755 | −0.1085 | −0.1065 | −0.2235 | 0.2795 | −0.0475 | 0.2565 | −0.8365 | −0.6885 | −0.4965 | −0.2655 |
| GSC | 0.47 | −0.088 | −0.64 | −0.834 | 0.456 | 0.022 | 0.001 | −0.365 | 0.409 | 0.08 | 0.465 | −0.953 | −0.174 | −0.018 | −0.67 |
| GSDML | −0.27 | −0.285 | 0.353 | 0.598 | −0.044 | 0.834 | 2.348 | 0.393 | 1.389 | 0.707 | −0.211 | −0.275 | 1.3 | −0.003 | −2.273 |
| GSK3B | 0.789 | 0.675 | −0.12 | 0.59 | 0.433 | −0.785 | 1.095 | −0.549 | 0.105 | 0.236 | −0.268 | −0.099 | 0.237 | 0.628 | −0.801 |
| GSTA1 | −0.2795 | −0.3855 | −1.4395 | −1.1345 | −0.4865 | −0.3415 | −1.3615 | −2.0615 | −1.3345 | −0.2805 | −2.6285 | −3.0645 | −0.8045 | −1.5335 | −1.2445 |
| GSTK1 | 1.176 | 0.423 | 0.655 | 1.056 | 0.947 | 2.031 | 0.875 | 0.661 | 1.129 | 0.604 | 1.374 | 0.872 | 1.353 | 1.088 | 0.488 |
| GSTM3 | −0.4075 | −0.4785 | −1.6925 | 0.0635 | 0.2925 | −0.2795 | 0.0075 | −1.4485 | −0.7555 | −1.2055 | −0.7235 | −2.0445 | −0.4045 | −1.8525 | −0.2785 |
| GSTP1 | 1.649 | −0.729 | −1.123 | −0.673 | −0.677 | −0.677 | 1.233 | 1.887 | 1.85 | 1.678 | −0.714 | −1.178 | 0.824 | −1.175 | −0.823 |
| GSTT1 | 0.0415 | 1.3595 | 1.4965 | 0.6485 | 0.8175 | 0.7575 | 0.2985 | −0.1895 | 0.2555 | 0.4785 | 1.6375 | 1.4345 | 0.1765 | 0.8245 | 1.3525 |
| GSTT2 | 0.1535 | −0.4035 | −0.1905 | −0.5295 | 0.5585 | −0.0795 | −0.0635 | 0.4235 | 0.6475 | 0.7175 | 0.0025 | 0.6485 | −0.0445 | 0.0225 | 0.2155 |
| GTF2F2 | −0.305 | 0.679 | 0.438 | 0.117 | 0.304 | 0.026 | −0.035 | −0.01 | −0.903 | −0.096 | 1.357 | 0.955 | −0.051 | 0.735 | 0.524 |
| GTF2I | −0.2235 | 0.6965 | 1.1445 | 0.8475 | 1.5475 | 0.8425 | −0.6925 | −0.9655 | 0.7195 | −0.6545 | 0.5175 | −1.8475 | 0.6935 | −0.9235 | 0.7765 |
| GTF3A | 0.741 | 0.696 | 1.546 | 0.681 | 2.271 | 1.453 | 0.414 | 1.22 | 0.117 | 0.876 | 0.279 | 1.847 | 1.369 | 1.264 | 1.384 |
| GTPBP3 | −0.233 | 0.255 | 1.054 | −3.24 | −1.127 | −0.42 | −0.386 | −0.004 | −0.62 | 0.002 | −0.223 | −1.19 | −1.465 | −2.084 | −3.152 |
| GTPBP4 | 0.575 | −0.108 | 0.817 | −0.118 | 0.496 | 1.259 | 0.893 | 0.124 | 0.725 | 0.092 | −0.021 | 1.413 | 0.587 | 0.251 | −0.204 |
| H1F0 | −0.8985 | −0.2825 | 0.1435 | −0.2305 | −0.5665 | −0.1385 | −0.7045 | −0.3235 | 0.1335 | 0.1245 | −0.0285 | −1.1115 | −0.1195 | −0.4565 | −0.2835 |
| H2AFV | 0.0075 | −0.5975 | 0.7185 | 1.0255 | 0.0765 | 1.0825 | 0.2815 | 0.3255 | 0.4015 | 0.5195 | 1.0295 | 0.8825 | 0.4226 | 0.4705 | 2.2155 |
| H2AFY | −0.1835 | 2.1276 | −0.8855 | 0.2155 | 0.5355 | 0.4925 | −0.1555 | 0.0265 | −0.8405 | −0.6355 | 0.7355 | 0.8005 | 0.7955 | 0.9735 | 0.6185 |
| H2AFZ | 0.1505 | −0.3415 | −0.8645 | −0.8855 | −0.0515 | 0.3545 | 0.7035 | 0.5325 | 0.5535 | −0.1935 | 0.1875 | −0.3575 | 0.0185 | 0.8145 | 0.6975 |
| H2BFS | 0.2815 | −0.1985 | 0.4645 | −0.2355 | 0.7915 | −0.1635 | 0.1065 | 0.2205 | −0.4445 | 0.0635 | −0.3625 | 0.2895 | 0.5885 | 0.1125 | −0.2925 |
| H3F3B | −0.446 | −0.412 | −1.58 | −0.353 | 0.151 | −0.425 | −0.904 | −0.57 | −0.592 | −0.384 | −0.514 | 0.021 | −0.457 | −0.691 | −1.302 |
| H41 | −0.7785 | −0.0715 | −0.4395 | −0.4195 | −0.0225 | −0.8795 | −1.0835 | −0.9845 | −0.4095 | 0.0225 | −0.6635 | −0.9525 | −0.1555 | −0.4845 | −0.6385 |
| HAGH | −0.994 | −0.615 | −0.263 | −0.761 | −0.572 | 0.579 | −0.292 | −0.386 | 0.006 | 0.169 | −0.513 | 0.344 | 1.377 | −0.148 | 0.01 |
| HBG1 | 0.5815 | −0.1845 | 1.1185 | 0.3915 | 1.3515 | 1.7615 | 0.9105 | 1.6805 | 0.9275 | 0.8045 | 0.3425 | 0.6495 | 0.4345 | 1.9565 | 0.6415 |
| HBL01 | 0.828 | 0.777 | 1.138 | 1.222 | 0.002 | −0.079 | −0.32 | 0.952 | −0.073 | −0.273 | 2.418 | 1.959 | 0.292 | 0.552 | 1.35 |
| HCAP-G | 0.7235 | −0.3185 | 0.1735 | 0.7195 | 0.5245 | 0.5715 | 2.1805 | 1.2435 | 1.1625 | 1.7985 | 2.5175 | 2.6485 | 1.1905 | 1.6645 | 0.1385 |
| HCP5 | −1.424 | −0.851 | −0.752 | −0.152 | −0.741 | 1.076 | 0 | −0.465 | −0.202 | −0.668 | −0.75 | −0.312 | −0.218 | −0.169 | −0.23 |
| HDAC2 | −1.194 | −0.261 | 0.022 | −0.237 | −0.257 | 0.298 | −0.554 | −0.401 | −0.114 | 0.322 | −1.512 | −0.556 | −0.499 | −0.555 | −0.126 |
| HDC | −0.1565 | 0.2445 | 0.3035 | 0.2045 | 0.0785 | −0.1355 | −0.0755 | 0.1715 | 0.6745 | 0.2315 | 0.1505 | −0.3125 | 0.2435 | −0.1755 | −0.1085 |
| HEBP1 | −1.01 | −0.424 | −1.081 | −0.768 | −0.415 | 0.528 | −1.422 | 0.244 | −0.548 | 0.033 | 0.705 | −1.435 | −0.544 | 0.276 | 0.226 |
| HIPK2 | −0.201 | −1.771 | −0.748 | −1.254 | 0.709 | −0.567 | −0.658 | −0.94 | 0.16 | −1.231 | −1.17 | −1.557 | −0.231 | 0.057 | −1.415 |
| HIPK2 | 1.0615 | 3.0735 | −0.0395 | 0.2175 | 0.9595 | 0.3005 | −0.2175 | 0.0275 | 0.3475 | 0.1115 | 0.4815 | 0.5945 | 0.0335 | 1.3835 | −0.6345 |
| HIS1 | −0.1405 | 1.8315 | −0.8885 | 0.0395 | 0.4155 | 0.1265 | −0.4185 | −0.3355 | −0.6995 | −0.8735 | 0.5565 | 0.0215 | 0.2815 | 0.2505 | 0.4895 |
| HIST1H2A | −0.334 | 1.714 | −1.187 | −0.183 | 0.101 | 0.002 | −0.577 | −0.326 | −0.858 | −0.627 | 0.565 | 0.056 | 0.359 | 0.254 | 0.479 |
| HIST1H2B | −0.4345 | 1.4695 | −1.2415 | −0.3775 | 0.0585 | −0.1135 | −0.5255 | −0.2455 | −0.5625 | −0.7245 | 0.5555 | 0.1065 | 0.1665 | 0.1455 | 0.3955 |
| HIST1H2B | −0.247 | 1.618 | −0.951 | −0.009 | 0.31 | −0.121 | −0.726 | −0.43 | −0.637 | −0.561 | 0.587 | 0.009 | 0.298 | 0.168 | 0.33 |
| HIST1H2B | 0.2015 | 0.2655 | −0.3075 | −0.3525 | −0.0655 | 0.0035 | 0.2485 | 0.0465 | 0.6495 | 0.2645 | 0.0005 | −0.8375 | 0.3835 | 0.6325 | 0.4075 |
| HIST1H2B | 0.6015 | −0.3385 | 0.2585 | 0.0065 | 0.5775 | 0.1525 | −0.3985 | 0.3155 | −0.3225 | −0.7365 | −0.1405 | −0.8305 | 0.1225 | 0.1025 | −0.1725 |
| HIST1H3C | 0.5085 | 0.1385 | −0.0695 | −0.4275 | 0.8735 | 1.1865 | 1.3225 | 0.2645 | 1.2935 | 0.8225 | 1.0855 | 0.6955 | 0.8275 | 1.3515 | 1.4945 |
| HM13 | 1.5865 | −0.8335 | 0.7165 | 0.0005 | 0.8775 | 1.1205 | 0.6065 | 0.5285 | 1.0185 | 0.1835 | 0.0425 | 0.1235 | 0.1005 | 2.1785 | 0.9015 |
| HMGB3 | 0.9735 | 0.0065 | −0.3815 | 1.1225 | 0.8255 | 0.9215 | 0.9255 | 0.5495 | 0.2625 | 0.6855 | 0.9065 | 0.7865 | 1.1785 | 0.5835 | 1.7105 |
| HN1 | 0.054 | −0.388 | 0.087 | −0.338 | −0.178 | 0.734 | 0.279 | −0.055 | 0.05 | −0.006 | 0.201 | −0.635 | 0.125 | 0.006 | 0.533 |
| HNRPA3P | −0.8625 | −0.0935 | 1.8945 | −0.1045 | −0.7865 | 1.1685 | −0.7025 | −0.5655 | 0.1475 | −0.2585 | −0.0425 | −0.4455 | −0.4885 | −0.7075 | −0.4615 |
| HNRPDL | −1.81 | −0.325 | 2.996 | 0.593 | −0.747 | 1.874 | −0.554 | −0.455 | 0.427 | −0.653 | 0.516 | −1.219 | −0.589 | −1.373 | −0.81 |
| HOXA5 | −1.7425 | −2.0355 | −0.0725 | −1.2745 | −2.5855 | −0.1685 | −1.8885 | −1.3055 | −0.5955 | −2.0895 | −3.2865 | −2.3445 | −1.8025 | −1.8665 | −2.1085 |
| HOXA7 | −0.7305 | −1.0855 | −1.6325 | −0.8445 | −0.7295 | −0.4475 | −0.5425 | 0.7695 | −0.4905 | −0.7325 | −2.0505 | −0.0516 | 0.4375 | −0.7285 | −0.1055 |
| HOXB2 | −1.1255 | 0.3215 | 0.7665 | −0.3405 | −0.2175 | −0.9835 | −1.2175 | −0.3905 | 0.2485 | −0.5845 | 0.1285 | 0.0335 | 0.0105 | −1.2175 | −0.2105 |
| HOXB6 | 0.5055 | −1.2785 | 0.0755 | 0.4835 | −2.0765 | 1.0315 | 0.1285 | −0.0515 | −0.0875 | −0.8255 | 1.0845 | −0.7525 | −0.5225 | −1.3455 | −0.8985 |
| HOXB7 | 0.9325 | −1.2885 | 0.0165 | 0.3715 | −1.6905 | −0.5305 | 0.6305 | 0.2716 | −0.3475 | −0.4125 | 1.5095 | 0.0545 | −0.0395 | −0.4825 | −1.4625 |
| HOXC10 | −0.532 | −3.253 | −0.421 | −0.801 | −2.72 | 0.24 | 0.426 | 0.16 | 0.533 | −0.793 | 0.069 | −0.814 | −0.774 | −2.28 | −0.867 |
| HOXC13 | −1.1485 | −0.4695 | 0.0325 | −0.3595 | 0.4085 | −1.3795 | 0.6085 | 0.1515 | −2.2615 | −1.7205 | 0.3975 | −1.8015 | −0.8885 | 0.5975 | −1.3595 |
| HOXC9 | −0.307 | −0.098 | 0.205 | 0.466 | 0.291 | 0.343 | −0.335 | −0.349 | −0.326 | −0.167 | 0.01 | 0.233 | 0.172 | −0.279 | −0.303 |
| HOXD3 | 0.3345 | −0.5565 | 0.6265 | −0.3045 | −0.2265 | −0.4015 | 0.7235 | 0.0135 | 0.0585 | −0.5195 | −0.2555 | 0.4695 | −0.7215 | 0.0085 | −0.3715 |
| HPCL2 | 1.288 | −0.457 | 1.516 | 2.13 | 2.406 | 0.174 | −0.12 | 0.258 | 0.508 | 2.486 | 1.133 | 2.685 | 0.261 | 1.47 | 0.443 |
| HPS3 | −0.2515 | −0.0175 | 0.0616 | 0.3025 | 1.2695 | −0.1815 | 0.4485 | 0.4475 | −0.1165 | 0.1245 | 0.2615 | 1.4445 | 1.7735 | 1.1745 | −0.3745 |
| HRASLS | 0.7085 | 0.8135 | 0.9675 | 0.5895 | 1.3405 | 1.4545 | 0.4535 | −0.2585 | −0.7405 | −1.0075 | 0.0855 | 1.1765 | 0.4715 | 0.9405 | 0.8015 |
| HRB | −0.038 | −1.497 | −0.704 | 0.492 | 0.962 | −0.331 | −0.853 | −0.025 | −0.218 | 4.228 | −1.147 | 0.214 | 0.835 | 0.936 | 2.852 |
| HRSP12 | 1.001 | 1.594 | 1.057 | −0.176 | 1.23 | 1.072 | 0.428 | −0.552 | −0.301 | −0.038 | −0.404 | 0.833 | 0.609 | 0.247 | 1.168 |
| HSD17B2 | 0.3915 | 0.1265 | 1.3765 | 1.3135 | 2.2835 | 1.3005 | −0.1325 | 0.6335 | 0.9255 | 0.8245 | 0.7645 | 1.0445 | 1.5585 | 1.0875 | 1.0075 |
| HSF1 | 0.8075 | −2.3065 | 0.0335 | −1.5435 | 1.2095 | 1.0275 | −0.5095 | −0.4155 | −1.0185 | −1.3195 | −0.7635 | −0.9335 | −0.4035 | −0.7285 | −1.4605 |
| HSPA14 | 0.791 | 0.453 | 0.901 | −0.99 | 1.304 | −0.753 | 0.748 | 0.861 | 0.571 | −0.749 | 0.437 | 1.248 | 1.351 | 0.448 | −0.528 |
| HSPA2 | −0.3135 | −1.0945 | −0.2635 | −0.0175 | 0.3165 | 0.0115 | 0.1515 | −0.4425 | −1.0955 | −1.0815 | −0.4305 | −0.4855 | 0.1775 | 0.4245 | −0.1065 |
| HSPA5BP [symbol] | −0.076 | 0.728 | 0.108 | 0.458 | 0.548 | 0.651 | 1.432 | 0.798 | 0.411 | 0.329 | −0.073 | 0.977 | 0.689 | 0.053 | −0.315 |
| HSPC016 | −0.511 | −0.075 | −0.391 | −0.003 | 0.494 | 0.359 | 0.719 | 0.336 | 0.335 | 0.83 | 0.349 | 0.449 | 0.388 | −0.151 | −0.14 |
| HSPC138 | −0.021 | −0.252 | 0.594 | 1.819 | 1.813 | 1.462 | 1.279 | 0.72 | 0.084 | 0.607 | 1.441 | 1.949 | 0.771 | 1.283 | 0.655 |
| HSPC148 | −0.4105 | 0.3185 | 0.2245 | 0.4505 | 0.5085 | −0.0525 | 1.3215 | 1.3435 | 0.6345 | 0.3535 | 0.3676 | 1.6445 | 0.7045 | 1.8415 | 0.7025 |
| HSPC150 | −0.1155 | 1.3705 | −0.2395 | 0.8435 | 0.5885 | 0.8955 | 0.9295 | −0.6045 | −0.0595 | 0.0255 | 0.3685 | 0.5825 | 0.2545 | 1.0425 | 0.0365 |
| HSPC163 | 1.002 | 1.018 | 1.967 | 0.909 | 1.504 | 2.165 | 0.684 | 0.528 | 0.809 | 0.212 | 0.084 | 1.142 | 1.056 | 0.906 | 1.463 |
| HSPC266 | 0.772 | 1.474 | −0.112 | 1.038 | 0.222 | 0.095 | −0.203 | −0.081 | 0.384 | −0.162 | 0.509 | 1.553 | 1.143 | 2.14 | 1.732 |
| HSU79274 | 0.931 | −0.093 | 0.425 | 0.246 | 0.576 | 0.962 | 0.585 | 0.192 | 0.859 | −0.554 | −0.583 | 0.394 | 1.431 | 0.799 | 0.96 |
| HTATIP2 | 0.473 | −0.387 | 0.496 | −0.553 | −0.416 | 0.079 | 0.331 | 0.169 | −0.348 | −0.361 | −0.089 | −0.041 | −2.024 | 1.23 | 0.038 |
| ICMT | 3.088 | 3.787 | −2.579 | 3.855 | −1.854 | 1.145 | 3.552 | 2.015 | 1.482 | 2.482 | 3.53 | −0.679 | −0.283 | −1.087 | −1.5 |
| ICT1 | −0.201 | −0.291 | 0.064 | 1.554 | 0.794 | 1.023 | 1.194 | −0.576 | −0.476 | −0.334 | 0.339 | 0.653 | 0.016 | 0.300 | −0.331 |
| ID4 | 0.6925 | −0.0895 | −0.6535 | −0.1615 | −0.8835 | −0.2915 | −0.8365 | −0.3545 | −0.2315 | −0.2835 | −0.4805 | 1.2715 | 0.1925 | −0.8076 | −0.6975 |
| IDH2 | 0.812 | 0.27 | 1.703 | 1.268 | 0.974 | 0.505 | −0.489 | 1.201 | 0.509 | −0.322 | 0.853 | 0.82 | 0.889 | 1.129 | 0.176 |
| IDS | 0.7135 | 2.0145 | 1.2745 | 1.2975 | 2.0105 | 0.4725 | −0.7705 | −0.1015 | 0.0585 | 0.9055 | 0.4095 | 0.8595 | 0.9875 | 1.5325 | 1.3355 |
| IFI30 | 0.5855 | 0.3745 | 0.0755 | 0.8125 | 0.7795 | 0.3125 | −0.8805 | 0.1275 | −0.5655 | 0.0055 | 0.0985 | 0.9875 | 0.3515 | 0.4445 | −0.1895 |
| IFI44 | 1.1845 | 1.0835 | 0.0435 | 0.6125 | 0.5775 | 0.2535 | −0.9955 | −0.4285 | −0.3595 | −0.2395 | −0.3305 | −0.4195 | 0.7265 | −0.0385 | 0.2685 |
| IFIT5 | −0.3255 | −1.1945 | −0.6495 | −0.6325 | −0.9305 | −0.4165 | −0.7405 | −0.9395 | −0.1875 | −0.1495 | −0.9935 | −1.0305 | −1.2985 | −0.3265 | −0.4345 |
| IFITM1 | −0.4215 | 0.4795 | −0.0035 | −0.3775 | −0.2965 | −0.2895 | −0.7125 | −0.7135 | 0.0035 | −0.3385 | −0.6295 | −0.8045 | −0.4835 | −0.5565 | −0.4075 |
| IGBP1 | 1.8845 | −1.6325 | −0.2975 | −2.2105 | −3.1025 | −0.0185 | 0.5335 | 0.2765 | −0.7495 | 0.2455 | 1.1095 | −1.6115 | −0.0115 | −2.0495 | −3.1475 |
| IGFALS | −0.2035 | 0.1616 | 0.9325 | 0.7955 | −0.3125 | −0.2865 | 0.0535 | −0.2845 | 1.0105 | −0.1055 | 1.4655 | −0.4475 | 0.9895 | 0.7985 | 0.2455 |
| IGPBP2 | −0.1685 | 0.2965 | 1.1685 | 1.0525 | −0.4285 | 0.3965 | 0.1735 | 0.7255 | 1.2585 | −0.1635 | 1.3325 | −0.2845 | 0.8725 | 0.2965 | 0.9255 |
| IGHG1 | −0.6175 | −2.0545 | 2.2355 | 0.4915 | −1.8645 | 0.0605 | −0.2465 | −0.3825 | 1.6655 | −1.4305 | −0.2035 | −0.7835 | 0.4925 | 2.3775 | 1.9815 |
| IGHG1 | −0.12 | −0.355 | 1.593 | 1.433 | −1.428 | 0.234 | 0.727 | 0.505 | 1.806 | −0.556 | 2.008 | −0.935 | 2.701 | 1.378 | 0.841 |
| IGJ | 0.119 | −0.253 | 1.059 | 1.045 | −2.383 | −0.337 | 0.025 | 0.445 | 0.87 | 0.271 | 1.587 | −0.883 | 1.532 | 0.794 | 0.378 |
| IGKC | 0.082 | 0.167 | 1.071 | 0.638 | −0.352 | −0.04 | −0.493 | −0.094 | 0.84 | −0.38 | 0.788 | −0.347 | 0.985 | 1.239 | 1.265 |
| IGKC | −0.587 | −0.626 | 1.271 | 0.128 | −1.33 | −1.269 | −1.142 | 0.45 | 1.277 | −0.474 | 0.096 | −0.88 | 1.759 | 1.274 | 0.141 |
| IGLC2 | −0.5605 | −1.7015 | −1.4115 | −0.9445 | 0.1525 | −0.3315 | −0.2675 | −0.0575 | −0.5565 | −0.8205 | −1.0515 | 0.0395 | −0.1165 | 0.1085 | −0.7585 |
| IGLL1 | −0.9085 | 1.6565 | −1.1295 | −0.0405 | −0.3735 | −0.0845 | 4.1375 | 0.6725 | −0.6805 | 0.6455 | −0.7855 | 0.1275 | −0.0205 | 0.8265 | −0.0025 |
| IL13RA1 | −0.611 | 0.24 | 1.592 | 0.225 | −0.485 | −0.115 | −0.036 | 0.757 | 2.582 | 0.43 | 0.127 | 0.52 | 0.781 | 0.773 | 2.778 |
| IL17B | −1.7565 | −1.1705 | −0.9875 | −0.2485 | −2.1125 | 0.1485 | −0.9285 | −1.1345 | −1.1685 | −1.8815 | −1.8485 | −0.8585 | −1.4905 | −1.7955 | −1.1295 |
| IL23A | 1.4905 | 0.2055 | 1.0795 | 0.7735 | 0.7465 | 0.5315 | 0.8765 | −0.6135 | 0.3245 | 0.7495 | 0.7895 | 0.5305 | −0.4595 | 0.6825 | 0.2105 |
| IL6ST | 2.4995 | 1.3085 | 1.0085 | 1.4115 | 1.8395 | 1.5785 | 1.0155 | 1.1315 | −0.3165 | 1.3135 | 1.4815 | 0.5465 | 1.3845 | 2.1465 | 1.0975 |
| ILF2 | −0.111 | −0.83 | 0.44 | −0.332 | −0.277 | 0.005 | 0.697 | −0.007 | −0.883 | −0.983 | 1.238 | −0.005 | −0.151 | −0.327 | 0.614 |
| IMPA2 | 0.8795 | −2.2305 | −3.2785 | −1.6205 | −2.4555 | −0.0815 | 1.7155 | 1.3445 | −0.9635 | 0.2975 | −1.4355 | 0.9425 | −2.8455 | −1.5195 | −1.2815 |
| ING1L | −0.933 | −1.857 | −2.19 | −0.895 | −2.392 | −0.725 | −0.758 | −1.562 | −1.535 | −1.383 | −2.265 | −0.395 | −1.997 | −1.434 | −1.685 |
| INHBB | −0.2935 | 1.3755 | −0.0545 | −0.4405 | −0.3435 | −0.4785 | −0.1895 | −0.0105 | 0.0385 | −0.0845 | −0.7985 | −0.1625 | −0.2415 | −0.2765 | 0.2835 |
| INPP4B | 6.1065 | −1.9245 | −1.9575 | −0.7155 | −1.3075 | 0.3885 | −0.1345 | −0.4415 | −0.8685 | −1.3735 | 0.1215 | −1.3625 | 1.4245 | −1.1695 | 0.9185 |
| INSM1 | −0.97 | −1.44 | −1.065 | −0.288 | −0.466 | −0.305 | −0.736 | −0.648 | −0.162 | −1.658 | 0.515 | −0.07 | −1.347 | −0.333 | −1.109 |
| IQCA | −1.693 | −2.224 | −0.658 | 0.87 | −1.77 | −0.369 | −1.528 | −1.392 | −0.268 | −1.907 | −1.333 | −1.195 | −1.478 | −1.218 | −0.305 |
| IQCE | −1.223 | −0.785 | −0.002 | 0.181 | −0.676 | −1.6 | −0.407 | 0.325 | −0.028 | −0.287 | 0.168 | −0.315 | 0.638 | −0.05 | 0.315 |
| IQGAP2 | −0.0776 | −1.2875 | 0.2025 | 1.1335 | −0.6285 | −0.4925 | 0.0415 | 0.9175 | 1.6385 | 0.0695 | 2.2495 | −0.5275 | 1.5985 | 1.4875 | 1.5815 |
| IRF4 | 1.8775 | −0.6305 | −1.2055 | −1.7795 | −2.7255 | 0.4575 | −1.6355 | 0.9995 | 2.0525 | 1.9135 | 2.9805 | 0.7805 | 0.1145 | −0.0155 | −0.3075 |
| IRTA2 | 0.827 | 0.499 | 1.245 | 0.831 | −0.222 | −0.855 | −1.145 | −0.191 | 0.213 | −0.547 | 0.222 | 0.847 | 0.448 | 0.736 | 0.726 |
| IRX4 | 1.4555 | −0.0075 | 0.5365 | −0.0585 | 0.8185 | 0.2505 | −0.6035 | −0.2375 | −0.8695 | 0.3205 | −0.0125 | −0.3275 | −0.4876 | −0.3735 | −0.2985 |
| ISG20 | 2.823 | 1.436 | 1.737 | 0.005 | −1.408 | −0.631 | 1.695 | 2.125 | 0.035 | 0.482 | 0.781 | −0.571 | −0.124 | 0.838 | 0.109 |
| ISGF3 [symbol] | −0.923 | −2.243 | 0.123 | −0.356 | −1.265 | −1.455 | −1.228 | −1.802 | −0.772 | −1.736 | −1.948 | −1.184 | −0.712 | −0.903 | −1.191 |
| ITGB4 | −1.217 | −0.005 | 1.204 | −0.388 | −0.609 | 0.002 | 1.292 | 0.232 | 0.556 | 0.787 | 0.97 | −0.749 | −0.002 | −0.541 | 1.732 |
| ITPR1 | −0.32 | −1.038 | −0.577 | −0.959 | −2.426 | −1.54 | −2.609 | −0.92 | −0.388 | −2.902 | −0.338 | −3.888 | 0.337 | 0.554 | −0.3 |
| JARID1A | 0.478 | −0.295 | 0.229 | 0.231 | 0.968 | 0.769 | 1.271 | 1.441 | 0.78 | 0.092 | 0.761 | −0.018 | 0.008 | 0.865 | 0.047 |
| KARCA1 | −0.258 | −0.68 | −0.019 | −0.322 | −0.838 | −0.18 | 0.419 | 0.158 | −0.504 | −0.016 | −0.127 | 0.279 | −0.068 | −0.323 | 0.076 |
| KATNA1 | −0.6995 | −0.7085 | −0.5115 | −1.0775 | 0.1765 | −0.2315 | −0.1505 | 0.2355 | 0.4625 | 0.0435 | −0.2285 | 0.0085 | −0.1735 | 0.0345 | −1.7595 |
| KCNC2 | −0.177 | 0.502 | 0.615 | 0.531 | 0.258 | 0.415 | 0.092 | 0.266 | 0.097 | 0.152 | 0.098 | −0.516 | 0.303 | −0.288 | −0.015 |
| KCND2 | 0.942 | 2.609 | 1.303 | 2.129 | 0.503 | 2.235 | 1.206 | 1.429 | 2.323 | 1.413 | 3.427 | 0.22 | 1.621 | 0.263 | −0.669 |
| KCNK4 | −0.385 | −1.912 | −2.906 | −1.373 | −2.028 | −0.211 | −0.935 | 1.022 | 0.107 | −0.942 | −0.333 | 1.969 | 1.706 | 1.088 | 0.564 |
| KCNN4 | 1.0615 | 1.5335 | −0.0225 | 1.1505 | −0.0025 | 0.3475 | 0.1195 | −1.2565 | −0.4555 | 0.7325 | 0.5825 | −0.4405 | −1.1225 | 0.4985 | 0.5855 |
| KCNS3 | −0.4085 | 0.6976 | −0.4725 | −0.4655 | −2.0535 | −1.0385 | 0.2846 | −0.3045 | −0.1465 | −0.4995 | −0.3485 | 0.2775 | −1.2205 | 0.3575 | −0.4685 |
| KCTD15 | 1.058 | 0.242 | −1.178 | 0.829 | 1.107 | 0.235 | 0.135 | 0.188 | 0.213 | −0.71 | −0.129 | 0.568 | 1.053 | −0.121 | −0.743 |
| KCTD3 | 1.343 | 1.508 | 0.873 | 1.715 | 1.788 | 0.305 | 0.594 | 0.723 | 0.452 | 0.434 | 1.032 | 1.217 | 1.44 | 1.363 | −0.103 |
| KDELR2 | −0.005 | −0.018 | 1.165 | 0.895 | 0.323 | 1.578 | 0.422 | −0.085 | 0.253 | 0.729 | 0.044 | 0.957 | 0.530 | 0.068 | 0.206 |
| KIAA0020 | −0.172 | 0.831 | 0.75 | 0.839 | 0.867 | 0.459 | −0.347 | 0.195 | −0.638 | −0.282 | 0.273 | −0.388 | −0.276 | 0.259 | −0.425 |
| KIAA0101 | −0.4835 | −0.4065 | −0.9505 | −1.3055 | −0.8575 | −0.2876 | 0.5305 | −0.2195 | −1.1505 | −0.4005 | −0.1536 | −1.0025 | −1.0715 | −1.0835 | −2.0825 |
| KIAA0153 | −0.0145 | 0.2675 | 0.5695 | 0.1216 | 0.6785 | −0.1135 | 0.9955 | −0.2335 | −0.8305 | −0.0975 | −0.1255 | 1.3305 | 0.0785 | −0.4425 | 0.0025 |
| KIAA0182 | −1.3205 | −0.9425 | 0.1035 | −0.4075 | 0.2025 | 0.0825 | 0.3325 | 0.4145 | 0.3135 | −0.2935 | −0.4716 | 1.3935 | 0.2795 | 0.9475 | 0.0535 |
| KIAA0196 | 0.2195 | −1.1345 | −0.2025 | −0.4275 | 0.1615 | −0.0055 | −0.6695 | −0.7585 | −0.5185 | −0.1055 | 0.1485 | 0.0055 | −0.4485 | 0.2135 | −0.2216 |
| KIAA0241 | 0.811 | −0.77 | −0.832 | −0.677 | −1.127 | 0.185 | 0.857 | −0.006 | −0.319 | 0.126 | 0.549 | 1.22 | 0.195 | 0.446 | −0.985 |
| KIAA0251 | −0.5835 | 0.1085 | −0.3965 | −0.0715 | 0.0535 | −0.0335 | −0.4075 | −0.2625 | −0.6125 | 0.1405 | −0.1985 | −0.2075 | −0.8415 | 0.4875 | −0.5905 |
| KIAA0274 | −0.595 | 0.894 | 0.054 | 0.534 | −0.48 | 0.467 | −0.422 | −0.918 | 1.138 | −1.053 | −0.013 | −1.484 | −1.314 | −0.97 | 1.32 |
| KIAA0515 | 1.0845 | 0.0635 | 0.4585 | 0.2555 | 0.6345 | −0.0535 | 1.0015 | 0.7885 | −0.6865 | −0.4985 | 1.0215 | 0.4065 | 0.3185 | 0.6855 | 0.4465 |
| KIAA0582 | 1.4105 | 0.6655 | 0.1665 | 0.5025 | 0.0635 | 0.9965 | 0.0595 | −1.3465 | −0.6875 | −0.6895 | −0.0045 | 0.2275 | 0.2465 | −1.5305 | −1.6325 |
| KIAA0650 | 0.4675 | 0.1305 | −1.1515 | −0.0285 | 0.7365 | 0.4515 | 1.5415 | 1.2095 | −0.0085 | 2.7775 | −0.3815 | 1.2215 | 0.4065 | 0.0975 | −0.4795 |
| KIAA0703 | −0.82 | 0.675 | −2.398 | −1.007 | 0.198 | −0.298 | −0.12 | 0.96 | 1.671 | 0.583 | 0.858 | 1.344 | −0.153 | 0.268 | 0.331 |
| KIAA0830 | −0.0885 | −1.9845 | −0.6015 | −0.6155 | −0.8585 | −1.0325 | −1.0265 | −3.8795 | −2.5015 | −0.7825 | 0.5065 | −4.4905 | −3.3675 | −2.2025 | −0.0185 |
| KIAA0980 | −0.2995 | −1.2005 | 1.3045 | 0.0325 | −0.6315 | −0.7275 | −1.1605 | −0.3925 | −0.3845 | −1.3895 | −1.2225 | 0.1475 | 0.2355 | −0.0565 | 1.2525 |
| KIAA1324 | −0.772 | −0.507 | −1.139 | −0.88 | −0.264 | 0.841 | 0.871 | −0.603 | −0.634 | −0.81 | −0.81 | −0.761 | 0.082 | −0.232 | 1.107 |
| KIAA1363 | 0.151 | −2.06 | −1.925 | −1.484 | −2.505 | −0.258 | −0.233 | −1.144 | −0.296 | 0.003 | −0.219 | 0.204 | −0.917 | 0.273 | −1.145 |
| KIAA1683 | 0.0695 | 0.0616 | −0.3085 | −0.4585 | 1.6905 | −1.1545 | 0.3795 | 0.8305 | 0.9745 | 0.6655 | 1.2255 | 1.1565 | 0.0045 | 1.2765 | 0.2755 |
| KIF12B | −0.5655 | 0.2115 | 0.6165 | −0.2235 | 1.1335 | 0.5815 | −0.1725 | 0.3785 | 0.2405 | −0.0275 | 1.0225 | 0.3235 | 0.9865 | 0.0275 | 1.8065 |
| KIF24 | 0.0825 | 0.2055 | 0.1775 | 0.1445 | −0.1265 | −0.4475 | −0.1345 | 0.0505 | 0.0015 | 0.2105 | 0.0475 | −0.1275 | 0.2615 | −0.3015 | 0.1876 |
| KIN | 1.9155 | −0.0955 | 0.8415 | 1.8215 | 2.4485 | 1.5005 | 0.1765 | −0.9975 | 1.8905 | 2.1195 | 2.0475 | 2.6285 | 1.4285 | 1.2645 | 4.0465 |
| KIRREL3 | 3.8735 | 3.1235 | −0.3705 | 0.4345 | 0.2716 | −0.1265 | 2.6315 | 1.8275 | 2.6065 | 2.9475 | 3.4715 | 0.4255 | 1.7595 | 1.4635 | −0.3815 |
| KIT | 8.8385 | 5.7815 | 5.8665 | 5.2955 | 3.2155 | −0.6565 | 1.8825 | 1.9855 | 3.6345 | 1.5825 | 3.8885 | 1.6505 | 5.6335 | 4.9985 | −0.1515 |
| KLK5 | 5.233 | 4.795 | 1.423 | 3.304 | 0.687 | −0.785 | 0.604 | 0.242 | 0.916 | 3.209 | 4.379 | 0.9 | 3.045 | 1.488 | −0.208 |
| KLK6 | 4.944 | 4.506 | 3.713 | 4.458 | 1.943 | 0.54 | 1.264 | 1.894 | 3.694 | 1.734 | 3.922 | 0.502 | 3.872 | 3.402 | −0.806 |
| KLK7 | 1.372 | 0.085 | 1.468 | 1.533 | 1.832 | 2.126 | 1.376 | 1.93 | 0.469 | 0.721 | 1.79 | 0.796 | 0.613 | 0.958 | 1.451 |
| KLK8 | 1.1505 | −0.2985 | 0.4445 | 0.0885 | 0.5525 | 0.3985 | 1.1245 | 1.1385 | 0.3595 | 0.3325 | 0.1105 | 0.9575 | 0.4915 | 1.7725 | 0.5475 |
| KNTC2 | 0.277 | 1.099 | 2.67 | −0.753 | −0.618 | 1.398 | −0.692 | 0.171 | −0.723 | −0.414 | −0.957 | −0.153 | 0.596 | −0.457 | 0.030 |
| KPNA2 | −0.1325 | −0.8085 | −0.3285 | 0.2835 | −1.4595 | 0.0305 | 0.1975 | −0.3095 | 0.3905 | −1.0575 | −0.7655 | −0.1785 | 0.8945 | 0.1816 | −0.2205 |
| KREMEN2 | −0.8915 | 1.7275 | 0.4225 | 2.6215 | 1.0655 | 0.5435 | 0.1415 | 0.4175 | 0.6575 | 0.7815 | 3.1535 | 1.8176 | 0.1395 | 0.2225 | 0.7145 |
| KRT10 | −1.4215 | 0.8305 | −0.1435 | 2.1415 | 0.8965 | 0.7565 | 0.2955 | 0.9475 | 0.9035 | 2.2065 | 2.9555 | 1.6535 | 1.0825 | 0.4865 | 0.8065 |
| KRT13 | 3.668 | 1.846 | 0.358 | −0.339 | 0.324 | 0.682 | 1.431 | 2.373 | 1.739 | 2.53 | 1.782 | 0.327 | 1.878 | 3.055 | −2.097 |
| KRT15 | −1.642 | −2.244 | −1.334 | −1.705 | −1.342 | −1.186 | −1.612 | −1.51 | −1.545 | −2.363 | −2.084 | −2.373 | −1.108 | −1.939 | −1.678 |
| KRT17 | 2.9355 | 3.4785 | 2.0745 | 2.6345 | 1.2995 | 1.9495 | 3.8775 | 3.4165 | 3.2735 | 3.5345 | 1.7725 | 3.2875 | 2.8725 | −1.2455 | −1.6915 |
| KRT18 | 4.32 | 0.473 | 4.074 | 3.454 | 0.681 | 0.829 | 1.13 | 2.884 | 3.547 | 0.051 | 1.905 | 0.849 | 3.116 | 3.125 | −2.551 |
| KRT23 | −0.5135 | −0.4845 | 0.3495 | −0.3365 | −0.1155 | 0.1425 | 0.1775 | −0.0455 | −0.1886 | −0.2825 | 0.1155 | 0.0245 | 0.1685 | −0.0365 | 0.2645 |
| KRT6B | −0.531 | −0.016 | −0.6 | −0.721 | −0.22 | −0.372 | −0.593 | −0.148 | 0.657 | 0.113 | −0.584 | −0.536 | 0.397 | −0.622 | 0.09 |
| KRTAP3-3 | 0.9695 | −1.8475 | −1.4555 | 0.4185 | −1.0095 | 0.7185 | −1.1075 | −0.8185 | −0.6575 | −0.9475 | −1.0895 | −1.9375 | 0.6765 | −0.7125 | −0.7685 |
| KRTHA7 | 0.8945 | 1.0345 | 1.7645 | 0.8545 | −1.7255 | 0.7235 | −0.5055 | 1.6745 | 1.9785 | 1.4645 | 1.3145 | 0.6645 | −0.2775 | −0.6505 | 2.1065 |
| LAMA3 | 0.234 | −0.060 | −0.417 | −0.001 | 0.287 | −0.803 | −0.278 | −0.399 | −0.781 | −0.367 | −0.447 | −0.436 | −1.195 | −0.275 | −0.708 |
| LAMB3 | −0.349 | 0.025 | 0.981 | 0.398 | −0.229 | 0.101 | 0.272 | 0.377 | 0.928 | 0.116 | 1.451 | −0.14 | 0.808 | 0.6 | 0.267 |
| LASS2 | 0.4685 | 0.4285 | 1.1555 | 1.1055 | 0.6816 | 0.7745 | 1.1385 | 1.3575 | 0.7595 | 0.8955 | 1.8795 | 1.9415 | 0.7665 | 2.1815 | 2.0915 |
| LAX | −2.1845 | −0.6525 | 0.7575 | −0.8005 | 0.4925 | −0.5125 | 1.2725 | 1.5275 | −0.9075 | −1.4725 | 0.0815 | −1.8505 | −1.7046 | 1.2915 | −0.7025 |
| LBR | 0.2795 | 1.1255 | 2.4715 | 1.0335 | −0.0755 | 1.5515 | 0.0515 | 1.0675 | 1.4675 | 0.8825 | 1.8535 | 0.1595 | 0.5995 | 1.4395 | 1.8205 |
| LCE1B | 0.0075 | 0.1785 | 0.4815 | 0.3545 | 0.0655 | 0.0885 | −0.0915 | 0.1715 | 0.3405 | 0.2615 | 0.1235 | −0.0245 | −0.3835 | −0.8025 | 0.0065 |
| LDHB | −1.288 | −0.452 | −0.737 | −0.677 | −0.422 | −0.103 | −0.863 | −0.834 | 0.087 | −0.156 | −0.551 | −1.237 | −0.54 | −0.633 | −0.531 |
| LDOC1L | −2.223 | −1.702 | −1.577 | −1.483 | −1.894 | 0.559 | −2.22 | −1.497 | 0.934 | −1.542 | −1.422 | −2.509 | −1.561 | −1.856 | −1.348 |
| LETMD1 | −2.0085 | 0.5035 | 0.5945 | 1.8455 | −0.0595 | 0.7155 | −0.2635 | −0.7805 | −0.7105 | −0.8075 | 1.2125 | 0.1115 | −0.7165 | 1.3435 | 1.5065 |
| LFNG | −0.263 | 0.785 | 0.33 | −0.021 | 0.005 | −0.414 | 0.243 | 0.103 | −0.165 | 0.848 | 0.58 | 0.468 | −0.075 | 0.631 | −0.45 |
| LGALS2 | −0.867 | −0.608 | −0.122 | −0.17 | −2.682 | −0.142 | 0.802 | −0.337 | −1.468 | 0.873 | 0.027 | −0.229 | −2.465 | 0.164 | −0.772 |
| LGTN | −1.742 | 1.095 | −0.419 | −0.913 | −0.841 | −1.518 | −0.58 | 0.133 | 0.334 | −0.975 | 0.392 | −1.443 | −1.138 | −1.787 | −1.116 |
| LNX | −0.1085 | −0.6285 | 1.6225 | 0.0895 | 0.4915 | −1.0545 | 0.3555 | −0.1985 | −1.1745 | −0.9145 | −0.5385 | 0.4855 | −0.4855 | −0.3425 | 0.3985 |
| LOC11247 | 0.231 | 0.08 | 0.031 | 1.165 | 0.34 | 0.777 | 0.883 | 0.407 | −0.298 | 0.077 | −0.08 | −0.128 | 1.221 | 0.354 | 2.921 |
| LOC11492 | −0.895 | −0.073 | −0.62 | −0.537 | −1.127 | 0.903 | −0.107 | −0.672 | −0.388 | −0.859 | 1.001 | 0.306 | 1.101 | 0.552 | 2.605 |
| LOC11497 | −0.1135 | 1.3365 | 1.7475 | 0.1075 | −0.7545 | 1.5125 | 1.2275 | 0.2785 | −0.2055 | 0.0225 | 0.6265 | 0.8555 | 0.0565 | −0.1575 | 0.5246 |
| LOC11621 | −1.7865 | −2.3815 | −2.0225 | −2.0165 | −1.9595 | −0.9245 | −0.8365 | −1.3175 | −1.2825 | −1.1095 | −2.5445 | −0.6885 | −0.0285 | −1.9015 | −0.8515 |
| LOC11644 | −2.4105 | −0.8665 | −3.1515 | 1.7985 | 0.0125 | 1.1595 | −0.4265 | −4.4525 | −4.2075 | −1.4765 | −2.8135 | −0.4875 | −1.8255 | 0.2915 | −4.1495 |
| LOC12022 | −0.2315 | −0.9065 | −0.6015 | −0.4785 | −0.7195 | −0.6115 | −0.6775 | −0.3585 | −0.3715 | −0.1165 | −0.4265 | −0.5585 | 0.0415 | −0.3805 | −0.0775 |
| LOC12422 | −1.0325 | 0.5825 | −1.4465 | 0.1245 | −1.0665 | −0.7485 | 0.7295 | −0.1985 | 0.1675 | 0.5305 | −0.9115 | −1.3455 | −0.8125 | −0.2825 | −0.0315 |
| LOC14654 | −1.875 | −1.104 | −0.938 | −1.088 | −1.272 | −0.7 | −0.56 | −0.88 | −1.409 | −0.957 | −2.091 | −0.755 | −1.241 | −0.479 | −0.928 |
| LOC15506 | 3.031 | 0.43 | 1.808 | −1.095 | 2.058 | −0.028 | −1.181 | 0.667 | 0.128 | 0.14 | −0.591 | 0.541 | 1.269 | 0.394 | 0.297 |
| LOC20189 | −1.6045 | −1.8755 | −1.5205 | 2.5685 | 2.3965 | 0.7095 | 0.6515 | 0.4865 | 0.2465 | −0.6385 | −0.6115 | 1.3945 | −0.4815 | 2.1395 | −0.7075 |
| LOC25398 | 0.022 | 2.166 | −0.46 | −0.892 | −0.172 | 0.485 | 2.024 | 0.358 | 0.546 | 0.576 | 1.441 | −0.476 | −0.112 | −0.345 | −0.628 |
| LOC33990 | −1.438 | −2.179 | −2.838 | −1.337 | −2.85 | −1.032 | −0.516 | −1.668 | −1.884 | −1.47 | −1.558 | −1.355 | −1.518 | −3.004 | −2.448 |
| LOC34817 | 0.9575 | 0.6675 | 0.6025 | 1.5135 | 0.3035 | 1.2385 | 1.3805 | −0.2645 | −0.3145 | −0.2235 | 0.9185 | 0.7405 | 1.8816 | −0.0315 | −0.2425 |
| LOC40045 | 0.548 | −0.438 | −1.292 | −0.416 | −0.747 | 0.52 | −0.059 | 0.468 | 1.22 | −0.579 | −0.356 | −0.888 | 1.34 | 0.92 | 0.75 |
| LOC44089 | 0.853 | 0.037 | 1.039 | 0.522 | 1.509 | 1.26 | 1.292 | −0.043 | −0.311 | −0.349 | −0.056 | 1.544 | 0.468 | 0.284 | 0.684 |
| LOC49386 | 0.008 | −1.711 | −1.044 | −1.419 | 0.142 | −0.926 | −0.053 | −1.157 | −0.698 | −1.667 | −0.831 | −1.445 | 0.818 | −0.526 | −1.053 |
| LOC51123 | 0.898 | −0.074 | −0.568 | −0.709 | −1.058 | −0.677 | 0.119 | 0.225 | −0.692 | −0.024 | −0.232 | 0.229 | −0.138 | 0.791 | 0.878 |
| LOC51161 | −0.0305 | −1.2815 | −1.3985 | −0.1145 | −1.7425 | −1.6595 | −1.5535 | −1.2555 | −1.4775 | −1.3275 | −2.0085 | −0.8085 | −0.9895 | −1.7695 | −1.6685 |
| LOC51321 | −1.4575 | 0.1505 | 0.4285 | −0.4235 | 0.0895 | −0.4595 | 0.0935 | −1.0045 | −0.1535 | 0.3905 | −0.0685 | −0.8235 | −0.7535 | −1.5535 | 1.4665 |
| LOC51760 | −0.4655 | 0.5955 | 0.4875 | 0.9065 | 4.4545 | 5.0335 | −0.0815 | −0.0025 | 0.3795 | 0.1935 | 1.6325 | 1.3535 | 2.6025 | 4.2325 | −0.2875 |
| LOC81558 | −1.542 | 0.021 | −1.346 | −0.552 | −1.135 | −0.456 | −1.046 | −1.049 | −0.673 | −0.543 | −1.203 | −1.137 | −1.325 | −1.152 | 0.622 |
| LOC81569 | 0.9085 | −0.7425 | −0.3085 | 0.8955 | −0.3025 | −0.9775 | −0.1165 | 0.1615 | 0.1035 | −0.5495 | −0.3325 | −0.7505 | 0.5745 | 0.6285 | 0.5985 |
| LOC90355 | −0.716 | 0.033 | −0.114 | 0.534 | 1.352 | 0.219 | −0.84 | −0.304 | −0.475 | 0.068 | −0.839 | 1.005 | −0.121 | −0.757 | 0.096 |
| LOC90701 | −2.1 | −2.334 | 1.15 | −1.346 | −0.29 | 0.759 | 0.629 | 1.873 | 0.348 | 0.501 | 1.385 | −2.370 | 0.686 | −1.635 | −0.098 |
| LONP | −0.8815 | −0.9145 | −0.8405 | −1.0405 | −0.7775 | −0.1945 | −0.3165 | −0.2485 | −1.1165 | −0.3865 | −0.2225 | 0.6555 | −0.9075 | −0.7955 | −0.0995 |
| LRAP | −2.0235 | −1.3545 | −1.8685 | −1.5105 | −0.7795 | −0.5845 | −0.4785 | −1.1745 | −1.0905 | −0.6245 | −0.9725 | −2.2618 | −1.6545 | −1.9785 | −0.8385 |
| LRBA | −0.5595 | 2.1525 | 1.0645 | 0.4775 | 0.2745 | 0.5425 | −0.0775 | 1.3535 | 1.8755 | 1.9165 | 1.9335 | −1.0345 | −0.9935 | −0.0025 | 0.7075 |
| LRIG1 | 2.0585 | 0.5915 | 2.0875 | 2.0565 | 2.6505 | 0.6025 | 1.8485 | 0.5455 | 0.9985 | −0.0195 | 1.1805 | 2.0005 | 2.3525 | 1.3575 | 2.6245 |
| LRP6 | −1.513 | −1.01 | −3.265 | −0.926 | −1.664 | −0.012 | −0.433 | −0.657 | 0.69 | −0.121 | −2.07 | −1.984 | −1.057 | −0.772 | −0.945 |
| LRP8 | −0.402 | −0.272 | −0.215 | 0.558 | −0.863 | −0.053 | −0.838 | −0.578 | −0.724 | −0.754 | −0.531 | −0.897 | −0.891 | −0.809 | −0.514 |
| LRRC17 | −9.1335 | −1.3316 | 0.5715 | −0.5725 | −0.7575 | 0.3845 | 0.5455 | 1.0655 | −0.0375 | −0.2125 | 0.0995 | 0.3795 | 0.7945 | 0.5255 | 1.0025 |
| LRRC2 | −0.1225 | 0.5235 | −0.8005 | 0.0395 | 0.3435 | 0.6525 | 0.3915 | 0.7675 | 0.6735 | 1.1545 | −0.0155 | 0.4445 | 0.1185 | 0.4935 | 0.5945 |
| LSM1 | −0.6355 | −0.4105 | 3.1595 | 2.2625 | −0.2315 | 0.7655 | −0.4715 | −0.1115 | −0.1175 | −0.4845 | 2.0915 | −0.4505 | −0.1525 | 0.0035 | 1.9615 |
| LSM8 | 0.513 | 1.08 | 4.327 | 1.996 | 0.804 | 2.888 | 1.146 | −0.097 | −0.106 | −0.188 | −0.125 | 3.127 | 0.548 | 3.538 | −0.09 |
| LTB | 0.719 | 0.658 | 2.587 | 1.847 | −0.189 | 0.928 | 0.979 | 1.727 | 0.804 | 0.268 | 1.812 | 2.143 | 1.735 | 0.523 | 0.79 |
| LY6D | 0.265 | 0.875 | 1.745 | 2.935 | 0.585 | 1.144 | 0.805 | 1.065 | 0.637 | 1.155 | 1.605 | 1.245 | 1.366 | 1.201 | 1.191 |
| LYN | −0.159 | 0.253 | −0.508 | −0.091 | −0.698 | 0.258 | −0.107 | −0.288 | −0.168 | −0.605 | −0.629 | 0.947 | 0.203 | −0.544 | −0.232 |
| MA02L1 | −0.471 | −0.088 | −0.286 | 1.285 | 0.378 | −0.732 | −0.549 | 0.287 | 0.038 | −0.491 | −0.092 | 0.296 | 2.872 | −0.284 | −0.31 |
| MADP-1 | −0.9425 | −0.4485 | −1.0275 | 2.3205 | −0.4605 | −1.5165 | −0.6045 | −0.2585 | −0.1125 | 0.4905 | −1.4505 | −0.2025 | 3.1565 | −0.4925 | −0.2465 |
| MAGEA12 | −0.6 | −0.058 | −0.393 | 1.528 | 0.463 | −0.83 | −0.649 | −0.098 | 0.46 | 0.055 | −0.675 | 0.153 | 3.13 | 0.27 | 0.14 |
| MAGEA2 | −0.9215 | −0.5615 | −1.0055 | 2.2895 | −0.5265 | −1.3475 | −0.2525 | −0.6085 | −0.3395 | −0.0735 | 0.5785 | 5.7185 | 4.5545 | −0.5605 | 1.4345 |
| MAGEA3 | 1.8095 | 1.0705 | −1.8315 | −1.2765 | −0.8975 | −0.3125 | −0.4645 | 1.0925 | 1.2325 | 1.1885 | 1.4845 | −0.3605 | 0.1545 | 1.1275 | 1.6725 |
| MAGEA4 | 0.517 | 1.555 | 1.818 | 0.082 | 1.225 | 1.297 | 1.97 | 1.186 | 0.749 | 0.454 | 1.269 | 1.893 | 1.732 | 0.482 | 1.151 |
| MAGED4 | 0.5625 | −0.1545 | 0.2645 | −0.7335 | 1.0515 | 0.2545 | −0.2405 | −0.3115 | −0.9075 | 0.2395 | 1.5335 | −0.3865 | −1.0705 | −1.0475 | −1.0775 |
| MAGI1 | 0.4595 | 1.3585 | 1.8535 | 0.5545 | −0.8725 | −0.1825 | 0.4945 | 1.0815 | −0.9655 | −0.4505 | 0.2545 | 1.4985 | 0.2235 | −1.0285 | 0.2535 |
| MAGI-3 | 0.038 | 0.017 | 0.411 | 0.893 | 0.274 | 0.257 | −0.003 | 0.494 | 0.695 | 0.063 | 1.037 | −0.6 | −0.243 | 0.181 | 0.52 |
| MAL2 | 0.832 | 1.357 | 0.875 | 0.005 | 0.542 | −0.537 | −1.151 | −1.338 | −0.91 | −1.67 | −0.457 | 0.155 | 0.158 | 1.094 | −0.983 |
| MAN2B1 | 0.6415 | 1.7355 | 3.8825 | 1.7415 | 1.5425 | 1.4275 | 1.3975 | 2.8835 | −0.1495 | −0.6415 | 3.0775 | 2.5855 | 1.9365 | −0.7165 | 0.2215 |
| MAPK8IP2 | −0.9765 | −0.5785 | 0.3415 | −0.0735 | −0.4325 | −0.0055 | −0.8905 | −0.2105 | 0.1015 | −0.2655 | −0.0305 | −0.4475 | 0.6975 | −1.1825 | 0.4516 |
| MARCO | −2.039 | −0.858 | −2.38 | −1.56 | −1.228 | −0.335 | −1.018 | −0.792 | −1.579 | −1.982 | 0.597 | −0.944 | −0.507 | −1.157 | −1.044 |
| MARLIN1 | 0.545 | −0.129 | 1.222 | 0.245 | 0.497 | 0.68 | 0.037 | 0.149 | 1.025 | 0.062 | 1.389 | 0.364 | −0.479 | 0.398 | 0.441 |
| MASS1 | 0.7375 | −0.2565 | 0.8585 | −0.1655 | −0.0555 | 0.3825 | 0.4875 | 0.3055 | 1.2485 | −0.2515 | 1.2235 | 0.4495 | 0.1895 | 0.2145 | 0.4795 |
| MCM2 | 2.6165 | −0.0005 | 3.2625 | 2.1915 | −0.4495 | 0.4735 | 0.8035 | −0.5815 | 1.1825 | 0.1995 | −0.3065 | 1.9785 | 0.6115 | 1.5855 | 0.5135 |
| MCM3 | −0.458 | −0.585 | −0.25 | −0.834 | −0.34 | −0.873 | −1.789 | −1.237 | −2.381 | −1.771 | −2.298 | −0.693 | −0.459 | 0.058 | −2.239 |
| MESP1 | 0.0425 | −0.2935 | −0.0015 | −1.0185 | 0.7825 | 0.5115 | 0.7955 | 0.3235 | 1.0815 | 0.5435 | 0.8265 | −1.3265 | 0.0285 | 0.3435 | 0.1225 |
| METRN | 1.7275 | 2.6455 | 1.8595 | 3.1585 | 1.1525 | 0.9985 | 0.4575 | 0.3775 | 0.1365 | 2.3745 | 2.5085 | 1.8315 | 2.2955 | 0.2195 | 0.5255 |
| MFAP2 | 0.979 | −0.12 | 0.02 | −0.527 | 0.464 | −0.53 | −0.426 | 0.079 | −0.415 | −0.221 | −0.03 | 1.807 | 0.475 | 0.37 | 0.081 |
| MFGE8 | 0.9835 | −0.0905 | −0.0445 | 0.4335 | 0.5985 | 0.1285 | −0.5425 | −0.7455 | −0.0005 | −1.0535 | 0.3675 | −0.9645 | 0.0185 | −0.1245 | −0.7816 |
| MGAT4B | 0.95 | 0.362 | 0.308 | 0.41 | 0.265 | −0.07 | −0.523 | −0.24 | 0.818 | 0.191 | 1.133 | −0.068 | 0.051 | 0.826 | 1.056 |
| MGC1091 [symbol] | −0.6875 | −1.1426 | 0.4005 | −1.2705 | −1.6575 | −1.8995 | −0.0415 | −2.9035 | −0.8645 | −1.3675 | −1.0105 | 0.2255 | −0.8465 | −2.1045 | −2.8595 |
| MGC1098 [symbol] | 1.4535 | 1.1235 | −0.0315 | 1.4505 | 1.5255 | 1.2855 | −0.2025 | −0.5365 | −0.0945 | −1.0015 | 1.3315 | −1.1755 | −0.2965 | 0.5365 | 0.8805 |
| MGC1124 [symbol] | −0.887 | −0.803 | 0.032 | −0.231 | −0.002 | 0.889 | 1.195 | 0.936 | 0.805 | 0.918 | −0.118 | −0.542 | −0.473 | −0.055 | −0.007 |
| MGC1127 [symbol] | −0.398 | −0.702 | 1.289 | −0.143 | 0.903 | 0.259 | 1.98 | 0.822 | 1.02 | 0.498 | 0.558 | 0.191 | 0.103 | 0.844 | 1.905 |
| MGC1312 [symbol] | −1.041 | −0.508 | −1.625 | −0.521 | −0.779 | −0.242 | −1.546 | −0.923 | −0.98 | −0.324 | −0.744 | −0.23 | −1.471 | −0.554 | −1.011 |
| MGC1318 [symbol] | −0.688 | −0.605 | −0.227 | −0.181 | −0.38 | 0.288 | −0.497 | −0.44 | −0.42 | −1.148 | −0.94 | −0.403 | 0.482 | 0.052 | −0.325 |
| MGC1414 [symbol] | −0.3265 | −0.3535 | 0.4625 | −0.1155 | 0.4595 | 0.3705 | −0.1125 | 0.8855 | 0.4616 | −0.0805 | 0.0625 | −0.0565 | 0.2575 | 0.5595 | 0.3545 |
| MGG1428 [symbol] | 0.044 | −1.328 | −0.681 | −0.565 | 0.068 | 0.197 | −0.785 | −0.266 | 0.047 | −0.324 | −0.128 | −1.273 | −0.169 | −0.701 | −1.049 |
| MGC1481 [symbol] | 0.009 | 2.066 | 2.135 | 0.038 | 2.244 | −0.376 | −0.221 | 0.159 | −0.119 | −0.177 | 0.971 | 1.759 | 1.349 | 1.805 | 1.800 |
| MGC1580 [symbol] | 0.5055 | 0.4455 | −0.2445 | 0.3315 | 1.1185 | −0.6245 | 0.6395 | 0.4705 | 0.0695 | 0.4125 | 0.3905 | −0.1195 | −0.0355 | −0.3145 | −0.4075 |
| MGC1663 [symbol] | 0.642 | 0.377 | 0.783 | 0.946 | 0.903 | 0.7 | 0.478 | −0.221 | 0.631 | −0.048 | 0.42 | −0.224 | 0.857 | −0.213 | 0.888 |
| MGC1873 [symbol] | 1.228 | 0.989 | 0.243 | 0.677 | 0.635 | −0.078 | 1.415 | 1.935 | 2.021 | 1.406 | 0.453 | 1.065 | 1.412 | 1.97 | −0.078 |
| MGC2053 [symbol] | −0.86 | 0.543 | −0.037 | 1.779 | −0.451 | −0.115 | −0.332 | −0.562 | 0.311 | 0.413 | 0.185 | −0.232 | −0.673 | −0.321 | 0.235 |
| MGC2398 [symbol] | −0.115 | 0.172 | 0.551 | 1.157 | 1.05 | 1.59 | 1.14 | 0.572 | 0.885 | 1.024 | −0.227 | 0.871 | 1.984 | 1.308 | 1.358 |
| MGC2610 | 0.007 | 0.788 | 1.953 | 1.02 | −0.887 | −0.003 | −0.403 | 0.014 | −0.102 | 0.197 | 1.241 | −0.317 | 2.218 | 0.499 | 0.496 |
| MGC2714 | 0.359 | 0.524 | 2.23 | 1.721 | −0.608 | −0.131 | −0.206 | 0.347 | 1.027 | −0.092 | 1.549 | −0.354 | 1.926 | 0.824 | 0.848 |
| MGC2716 [symbol] | 0.4095 | 0.6385 | 1.8825 | 1.4245 | −0.8625 | −0.3595 | −0.1975 | 0.5275 | 1.1595 | −0.2205 | 1.1575 | −0.4075 | 2.1555 | 0.9045 | 1.2835 |
| MGC2716 [symbol] | 0.0775 | 0.0005 | 1.8695 | 1.7555 | −1.0305 | −0.1995 | 0.3905 | 1.1045 | 0.7755 | −0.5495 | 1.7045 | −0.3245 | 2.1595 | 0.5185 | 0.5505 |
| MGC2716 [symbol] | −0.13 | −0.189 | 1.457 | 1.059 | −1.108 | −0.049 | −0.032 | 0.558 | 0.823 | −0.166 | 1.338 | −0.218 | 2.168 | 0.886 | 0.737 |
| MGC2716 [symbol] | 0.0185 | 1.3315 | 0.4825 | 0.8875 | 0.5735 | 0.1895 | 1.4075 | 1.6765 | 0.6145 | 0.6745 | 1.4235 | 2.3045 | 0.4165 | 1.2755 | 0.8095 |
| MGC2716 [symbol] | −1.313 | −0.365 | −0.648 | −0.289 | −1.126 | −0.433 | −0.755 | −0.664 | −0.508 | −1.875 | −1.54 | −1.547 | −1.03 | 0.275 | −0.818 |
| MGC2887 [symbol] | 0.603 | 1.409 | 0.584 | 1.004 | 0.99 | −0.333 | 0.578 | 0.429 | 0.518 | −0.78 | 0.011 | 0.795 | 0.193 | 2.03 | 1.257 |
| MGC3212 [symbol] | 2.288 | 2.342 | 2.719 | 0.932 | 0.03 | 1.626 | 1.324 | −0.129 | 1.127 | 0.126 | 0.302 | 0.934 | 1.358 | 0.936 | 0.903 |
| MGC3321 [symbol] | 0.2455 | −0.4015 | 2.5805 | 2.8255 | 0.6415 | 0.0755 | 0.4325 | 0.8075 | 0.4815 | 1.3715 | 0.9885 | 0.4815 | 1.2555 | 1.0755 | 0.7525 |
| MGC3484 [symbol] | −0.6135 | 0.5325 | 0.6345 | −0.2455 | 0.6845 | 0.2435 | −0.1515 | −0.1855 | −0.3025 | 0.0565 | 1.0335 | 0.8615 | −0.7616 | −0.5305 | 0.4785 |
| MGC3492 [symbol] | −0.947 | −0.527 | −1.124 | −1.08 | −0.088 | −0.282 | −0.786 | −0.719 | 0.212 | 0.085 | 0.383 | −1.402 | 0.001 | −0.574 | −0.568 |
| MGC4021 [symbol] | 0.67 | 0.458 | 0.104 | 0.139 | 1.305 | 0.588 | 1.479 | 0.927 | 0.682 | 0.165 | 0.747 | 1.955 | 0.978 | 0.721 | −0.408 |
| MGC4251 | 0.3085 | −0.7075 | −0.3795 | −0.2355 | 0.1395 | −0.6775 | −0.5475 | 0.0795 | −0.3975 | −1.0725 | −0.0815 | −0.7655 | −0.3655 | −0.1155 | 0.2395 |
| MGC4308 | −0.295 | −0.731 | −0.086 | −0.182 | −0.228 | 2.013 | 0.384 | −0.361 | −0.583 | −0.005 | 0.58 | −0.707 | −1.109 | −1.838 | −1.285 |
| MGC4800 | 4.6295 | 4.1435 | 3.3705 | 1.8025 | −0.9565 | 2.5645 | 1.2605 | 2.7675 | 2.0945 | 3.4035 | 5.5585 | 3.1455 | 0.5045 | 0.0345 | 1.8445 |
| MGC4659 | 1.065 | 1.288 | 0.017 | 0.455 | −0.578 | 0.769 | 2.777 | 1.184 | 2.484 | 1.787 | 0.271 | 1.61 | 0.2 | 0.526 | 1.43 |
| MIA | 1.4915 | 1.2315 | 0.0095 | 0.5745 | −0.4385 | 1.0125 | 3.5805 | 1.4725 | 2.3045 | 1.7355 | 0.4375 | 1.9565 | 0.3755 | 0.6185 | 1.6565 |
| MID1 | 1.0985 | 1.0235 | 0.0545 | 0.4335 | −1.1985 | 1.3605 | 4.3355 | 1.9535 | 3.3935 | 1.8715 | 0.3125 | 3.0735 | 1.2925 | 1.0445 | 2.8145 |
| MID1 | 0.347 | −0.348 | 2.021 | 1.051 | 0.584 | 0.438 | 0.087 | 0.572 | −0.139 | −0.324 | 2.33 | 1.922 | −0.087 | 0.438 | 0.486 |
| MID1 | −0.7235 | −1.0915 | −0.5325 | 0.7155 | −0.6576 | 0.4395 | −0.6755 | 0.8305 | −0.2065 | −0.1695 | 1.4385 | 0.7005 | 0.2385 | −0.5125 | −0.4925 |
| MLF1IP | −0.0065 | 3.2885 | 2.0515 | −1.4675 | 4.9855 | −1.3365 | 4.8735 | 3.8855 | 1.0225 | 0.7475 | 0.1395 | 1.0125 | 2.9295 | 0.6545 | 0.5355 |
| MLSTD1 | 1.083 | 1.623 | 2.485 | 1.756 | 0.728 | 2.581 | 1.218 | 1.668 | 2.48 | 1.203 | 2.029 | 1.797 | 0.836 | 0.929 | −0.048 |
| MMP1 | 0.15 | −0.281 | 0.135 | −0.594 | −0.157 | 0.539 | 0.521 | −0.037 | −0.099 | −0.069 | −0.404 | −0.161 | 0.398 | −0.386 | −0.609 |
| MMP7 | 0.1725 | 0.1165 | 0.8745 | 1.0495 | 0.2275 | 0.1045 | 0.4855 | 0.3745 | −0.0935 | −0.3565 | 0.1455 | 0.8985 | 0.1995 | 0.4475 | 0.1935 |
| MNAT1 | 0.6045 | 1.0065 | 0.6425 | 0.0575 | 2.0475 | 2.5555 | 0.9415 | 0.0095 | 0.2885 | 0.0385 | 0.9235 | 0.4365 | 0.8385 | 1.4065 | 0.9035 |
| MRPL14 | −0.68 | −0.723 | −0.598 | −1.095 | −0.305 | −0.214 | 0.877 | −1.005 | 0.18 | 0.303 | −0.166 | −1.328 | −0.217 | 0.099 | −0.541 |
| MRPL15 | 0.5765 | 0.7315 | 0.3245 | 0.6615 | 0.7355 | −0.0565 | 0.1315 | −0.8385 | −0.8035 | −0.4095 | −0.3895 | −0.2725 | 0.2735 | −0.1045 | 0.9785 |
| MRPL27 | 0.0945 | −0.9585 | 0.0555 | −0.1065 | −0.1685 | −0.1755 | −0.2755 | −0.5515 | 0.3665 | 0.2795 | −0.0085 | −0.8385 | −0.9105 | −0.4075 | −1.1615 |
| MRPL34 | −0.931 | −0.599 | 0.121 | −0.315 | −0.068 | −0.037 | −0.531 | 0.12 | 0.515 | 0.485 | −0.705 | 0.343 | −0.892 | 0.079 | −0.709 |
| MRPL43 | 0.1555 | 0.5735 | 0.1995 | 0.2925 | 1.1905 | 0.4955 | 0.7645 | 0.6815 | 0.7815 | 0.8655 | 0.8925 | 0.3205 | 1.0655 | 0.3595 | 0.2285 |
| MRPL45 | 0.755 | −0.5 | 0.422 | 0.132 | 0.747 | −0.058 | 0.569 | 0.538 | 0.279 | 0.23 | 0.279 | 0.67 | −0.28 | 1.016 | 0.272 |
| MRPL48 | −0.6155 | −0.0225 | −0.2435 | 0.8935 | 0.7415 | 0.1055 | 0.3485 | −0.0275 | −0.0165 | 0.0345 | −0.1275 | 0.5625 | 0.5295 | 0.6485 | 0.2155 |
| MRPL9 | 0.0145 | −0.2115 | −0.2735 | −0.0005 | 0.3185 | −0.2995 | −0.4785 | −0.1125 | −0.0375 | −0.1455 | 0.1785 | −0.8765 | 0.4795 | 0.6835 | 0.1905 |
| MRPS14 | 0.416 | 0.899 | 1.284 | 0.593 | 1.316 | 0.771 | 0.563 | 0.387 | 0.515 | −0.207 | 0.112 | 0.47 | 0.838 | 0.382 | −0.295 |
| MRPS15 | 1.098 | 0.569 | 1.054 | 0.006 | 1.14 | 0.444 | −0.126 | −0.64 | 0.587 | 0.154 | 0.112 | 0.468 | 0.985 | 0.795 | −0.183 |
| MRPS17 | 0.649 | 0.212 | 0.347 | 0.789 | 1.819 | 0.683 | 0.328 | −0.104 | −0.13 | 0.06 | 0.001 | 0.996 | −0.873 | 1.247 | −0.495 |
| MRPS24 | −1.0075 | 0.2875 | 0.5515 | −0.4985 | 0.2345 | −0.1875 | 0.2345 | −0.0715 | 0.0615 | 0.3855 | 0.4375 | 1.1795 | −0.3545 | 0.2575 | −0.0355 |
| MRPS25 | 0.328 | 0.207 | −0.186 | 0.139 | 0.394 | 0.082 | 0.154 | −0.392 | 0.647 | −0.625 | −0.459 | −1.063 | 0.582 | 1.797 | 0.691 |
| MRPS31 | 0.125 | −0.204 | 0.740 | 1.365 | −0.219 | −1.238 | −0.323 | −0.306 | 0.198 | 0.275 | 1.042 | −0.312 | 0.041 | 0.03 | 0.175 |
| MRPS6 | 0.9355 | 0.3765 | 0.7445 | 0.3075 | 0.6935 | 0.8005 | 0.8875 | 0.5155 | 0.1645 | 0.6495 | 2.0075 | 1.0405 | 0.5035 | 0.6075 | 1.4665 |
| MS4A1 | −1.0475 | −0.4165 | −1.3605 | −0.0255 | −0.4555 | 0.2645 | −0.1735 | −0.0295 | 0.1255 | −0.0625 | −0.3565 | −0.6025 | −0.5205 | −0.1605 | 0.0345 |
| MSH2 | −0.943 | −1.217 | 0.903 | 0.239 | 0.009 | −1.782 | −0.569 | −0.571 | 0.268 | −0.477 | −1.4 | −0.648 | 0.073 | 1.583 | −0.132 |
| MSH3 | −0.326 | 0.083 | −0.093 | −0.03 | −0.355 | −0.544 | −0.331 | −1.06 | −0.267 | −0.039 | −0.008 | −1.962 | −0.373 | −1.144 | −0.623 |
| MSMB | 0.301 | −0.456 | −0.205 | −0.9 | −0.069 | −0.61 | −0.799 | 0.093 | −0.208 | −0.477 | 0.279 | −0.174 | −1.124 | 0.125 | −0.249 |
| MTA1 | 1.6815 | −0.6275 | −0.2775 | 1.5505 | 0.1805 | 0.8365 | 0.8175 | −0.7115 | −0.3865 | −1.4475 | −0.2215 | 0.5185 | −0.8215 | 1.8915 | −0.1855 |
| MTA3 | 0.391 | 0.214 | −0.088 | −0.169 | −0.224 | 0.524 | 0.033 | 0.649 | 0.071 | −0.347 | 0.675 | 0.65 | 1.049 | 0.999 | 0.103 |
| MTAC201 | 1.052 | −0.303 | 0.425 | 0.948 | 0.579 | 0.729 | 0.234 | −0.154 | −0.09 | 0.176 | 0.365 | 0.146 | −0.535 | 0.129 | −0.122 |
| MTCH1 | −0.1395 | 1.4835 | 0.7075 | 2.2915 | 1.6155 | 1.3235 | 1.6735 | 1.2555 | 1.5345 | 2.2585 | 0.8595 | 1.9445 | 1.5195 | 0.3075 | 0.7025 |
| MTHFD1 | 1.2265 | 0.0235 | 1.1225 | 0.9015 | −0.1165 | −0.5205 | −2.0595 | −1.9385 | −2.0745 | −3.3305 | −1.4095 | −0.8495 | −1.1335 | 0.6275 | −3.8535 |
| MTMR2 | −0.1175 | −0.3125 | 1.3255 | 1.4045 | 0.1585 | −0.6005 | −0.2075 | −0.0395 | 0.4375 | 1.8385 | 1.0935 | 0.5565 | 1.4045 | 2.3685 | −0.0885 |
| MUC1 | −1.337 | −1.076 | −0.147 | −0.944 | −0.314 | −0.322 | −1.215 | −0.555 | 0.343 | −0.494 | −0.087 | −0.962 | −1.378 | −1.446 | −1.222 |
| MUC20 | 0.759 | 1.478 | 0.049 | −0.481 | −0.063 | −0.144 | 0.198 | 0.488 | −0.063 | 1.119 | −0.408 | 0.079 | −0.72 | −0.485 | 0.316 |
| MUM1 | −2.849 | −1.496 | −2.065 | −2.88 | −1.295 | −0.663 | −2.928 | −2.212 | −2.362 | 1.76 | 1.554 | −3.444 | −1.689 | −2.826 | −0.553 |
| MXI1 | 1.125 | 1.539 | 0.688 | 1.274 | 2.891 | 2.789 | 3.958 | 2.44 | 0.168 | 0.794 | 0.75 | 2.565 | 1.898 | 0.008 | 2.888 |
| MYB | 0.617 | −0.313 | 1.486 | 1.13 | 1.803 | 1.357 | 0.046 | 0.708 | 1.073 | 0.571 | 0.915 | 0.846 | 0.459 | 0.66 | 1.188 |
| MYBL1 | 1.368 | 1.264 | −0.293 | 0.359 | 1.466 | 0.101 | 1.208 | 1.071 | 0.815 | 0.835 | 1.186 | 0.844 | −1.068 | 0.031 | −0.541 |
| MYBL2 | 0.078 | −1.167 | 0.41 | 0.944 | 0.985 | −0.625 | −0.066 | 0.058 | 0.069 | 0.421 | −0.028 | −0.335 | −0.19 | −0.238 | −0.477 |
| MYO10 | −0.0135 | −1.5035 | 0.3795 | −0.2475 | 1.3795 | −0.2935 | −0.0455 | 0.2135 | −0.2735 | 0.2295 | 0.6045 | −0.1855 | −0.4135 | −0.5055 | −0.0935 |
| MYO6 | 0.7555 | 1.5505 | −1.5115 | −1.3795 | 2.8235 | −0.3295 | 1.7075 | 1.7665 | 1.8115 | 2.2985 | −2.6815 | −1.4605 | 3.0405 | 1.9015 | 1.1405 |
| MYST3 | −0.3335 | −0.4115 | −0.3395 | 0.0795 | −0.1535 | −0.2235 | −0.2405 | −0.1415 | 0.0715 | −0.0805 | 0.2045 | −0.1025 | 0.2355 | −0.1565 | −0.1875 |
| NALP2 | 0.3185 | −0.2335 | 0.8485 | 0.0795 | −0.2765 | −0.3055 | −0.4575 | −0.4065 | −0.5815 | −0.6985 | −0.4325 | 0.1955 | −0.0355 | −0.4225 | 0.1425 |
| NALP6 | 0.4575 | −0.0585 | 0.0385 | −0.3715 | 0.2205 | 0.3205 | 0.5825 | 0.2575 | 0.5395 | 0.6516 | 0.5925 | −0.9735 | 0.0005 | 0.6865 | 0.5775 |
| NANS | −0.378 | −0.863 | −0.768 | −0.327 | −0.59 | −0.888 | −0.516 | −0.609 | 0.228 | −0.262 | −0.382 | −0.585 | −0.358 | −0.41 | −0.038 |
| NARF | 0.982 | 1.445 | −0.764 | −0.245 | −1.052 | −0.496 | −0.693 | −0.041 | −0.185 | 1.607 | 0.773 | −0.352 | 0.829 | 0.461 | 1.325 |
| NAT2 | −0.6865 | −1.1485 | 0.5845 | −0.2435 | 0.0365 | −0.3355 | −0.4705 | −0.2535 | −0.0485 | −0.0115 | 0.5075 | −0.3885 | −1.0695 | −0.7116 | 0.3825 |
| NAV2 | −0.48 | −1.456 | −0.625 | −0.898 | −0.152 | −0.787 | −0.278 | −0.706 | −0.288 | −0.606 | −0.542 | −0.327 | −0.326 | −0.691 | 0.067 |
| NDE1 | 1.2875 | 1.5035 | 1.7445 | 1.3145 | 1.7745 | 1.7475 | 0.6835 | −0.4055 | 0.1935 | 1.2075 | 2.4035 | −0.5595 | −0.6375 | −0.4215 | 0.2565 |
| NDP52 | 0.04 | 0.559 | 0.488 | 0.422 | 0.09 | 0.159 | −0.317 | −1.053 | −0.812 | 0.06 | 0.061 | −0.734 | 0.409 | 0.48 | −0.479 |
| NDRG2 | 0.329 | −0.391 | −0.02 | 1.065 | −0.361 | 0.659 | −0.318 | −0.741 | −0.975 | −0.285 | −0.453 | −0.578 | −0.789 | −0.305 | −0.733 |
| N0UFA7 | −0.0795 | −0.5925 | 0.3965 | −0.3905 | −0.2255 | 0.0785 | −0.8435 | −0.4905 | −0.1645 | −0.6345 | −0.1475 | −1.1085 | −0.4075 | 0.9835 | −0.7885 |
| NDUFB1 | 0.173 | 0.368 | −0.714 | 0.108 | −0.319 | 0.494 | 0.343 | −0.271 | 0.025 | 0.338 | −0.094 | 0.583 | 0.685 | 1.01 | 0.343 |
| NDUFB10 | −0.352 | 0.65 | 0.388 | 0.653 | −0.002 | −0.002 | 1.641 | −0.402 | 0.784 | 0.26 | 0.084 | 1.165 | 0.348 | 0.889 | 1.095 |
| NDUFB2 | 0.9695 | −0.0625 | 0.1185 | 0.5905 | 0.8335 | 0.3135 | −0.0575 | −0.4915 | −0.1305 | −0.2785 | 0.0905 | 0.8975 | 0.4205 | 1.3115 | 0.1105 |
| NDUFB5 | −0.1385 | −0.1605 | −0.9415 | 0.1985 | 1.3095 | 0.5215 | 1.0185 | 0.4455 | 0.0655 | −0.1405 | −0.0475 | 0.2385 | 1.2685 | 0.6705 | 0.1336 |
| NDUFB6 | −0.1915 | −0.2095 | 1.2145 | 0.5876 | −1.1245 | 0.7085 | 0.2495 | −0.8125 | −0.5365 | −0.5465 | −0.4285 | 0.5835 | 0.3565 | −0.2815 | 0.9245 |
| NDUFC2 | 0.2215 | 0.4045 | 1.4215 | 0.8435 | 1.3895 | 1.5245 | 1.3045 | 1.5005 | 0.3815 | 0.2035 | 1.1715 | 1.8725 | 0.1165 | 1.0375 | 0.8376 |
| NEBL | −0.551 | −0.174 | −0.26 | 0.597 | −0.114 | −0.006 | −0.66 | −0.405 | 0.013 | −0.187 | −0.416 | 1.053 | 0.282 | −0.174 | −0.515 |
| NEK2 | −1.443 | −1.402 | −1.483 | 0.124 | −1.75 | 1.899 | −0.624 | −0.65 | −0.23 | −0.915 | 0.101 | −0.887 | 0.173 | −0.788 | −0.226 |
| NEK6 | −1.2275 | −0.3895 | 0.3575 | −0.4345 | 0.3325 | −0.6215 | 0.3145 | 0.1745 | −0.1845 | −0.4535 | 0.8395 | −1.4495 | −1.0865 | 0.5945 | −1.0345 |
| NELL2 | 0.3585 | −0.4025 | 1.1375 | 0.9125 | 1.9145 | 0.7445 | 1.8395 | 1.5935 | 2.1745 | −0.7385 | 1.0405 | 1.7385 | 1.1745 | 2.1745 | −0.3755 |
| NEURL | −1.35 | −0.49 | −0.009 | 0.738 | −0.128 | 0.038 | −1.873 | −0.845 | −1.335 | −2.38 | −1.163 | −0.017 | −1.357 | −1.171 | −2.188 |
| NFE2L3 | 1.8105 | 2.1265 | 1.5325 | 0.6655 | 0.7585 | 1.0755 | 0.7865 | 0.2495 | 0.8485 | 2.2045 | 2.9375 | 0.7135 | 0.7076 | 0.9685 | 1.7025 |
| NFIA | −0.125 | 0.481 | 1.048 | −0.388 | −1.375 | −0.074 | −0.928 | −0.563 | −0.728 | −1.017 | 0 | −0.005 | −0.38 | −1.344 | −0.093 |
| NFIB | 0.6725 | 0.3185 | 1.2405 | 0.6095 | −0.0005 | 0.7995 | 0.1485 | −0.0985 | 0.4755 | 0.3875 | −0.2115 | 1.5645 | −0.6525 | 0.8035 | 0.7135 |
| NINJ1 | −0.1105 | 0.2625 | 0.0135 | 0.3165 | 0.1255 | −0.2576 | −0.3205 | −0.3115 | 0.0545 | −0.0776 | −0.0475 | −0.4395 | 0.2725 | 0.0015 | −0.0845 |
| NIPSNAP [symbol] | −0.3785 | 0.6185 | −0.0895 | −0.4575 | 0.3715 | −0.0495 | 1.5595 | −1.4465 | −0.1765 | 0.6755 | 0.5585 | −0.7545 | 0.4815 | 0.4805 | 1.2005 |
| NKX8-2 | −0.4985 | 0.5255 | −0.3225 | −0.6585 | 0.1395 | −0.0415 | 1.5445 | −2.7355 | 0.7155 | 0.9025 | 0.5935 | −1.0335 | 0.5505 | 0.4465 | 0.2405 |
| NME1 | −0.829 | −0.681 | −0.548 | −1.236 | −0.639 | −0.734 | −2.068 | −1.517 | −0.851 | −0.88 | −1.012 | −1.983 | −1.193 | −0.546 | −1.239 |
| NME2 | 1.7265 | 0.2195 | 0.9905 | 1.2185 | −0.8285 | −0.9485 | −0.9215 | −0.0095 | −0.1405 | −2.5275 | −0.2315 | 0.2795 | 0.8775 | 1.0825 | −1.1365 |
| NME3 | 0.7005 | 0.7085 | 1.0055 | 1.4365 | 0.5175 | 1.0905 | 0.5475 | 1.2405 | 0.9205 | −0.1455 | 0.5015 | 1.9335 | 1.4205 | 1.3705 | 1.3505 |
| NMES1 | 0.51 | 0.92 | 0.536 | 0.85 | 0.208 | 0.915 | 0.762 | 0.307 | 0.23 | −0.02 | 0.444 | 0.725 | 1.412 | 0.437 | 1.603 |
| NMI | 0.4855 | 0.3185 | 0.4395 | 0.1135 | −0.1515 | 0.2175 | 0.0595 | 0.7225 | 0.4395 | 0.3575 | 0.9045 | 0.3675 | 0.0185 | 0.4275 | 0.3285 |
| NOC4 | −0.2675 | 0.1095 | 0.8605 | 0.7375 | −0.3885 | 0.3645 | 0.0275 | 0.2255 | 0.7045 | 0.0145 | 1.1745 | −0.3816 | 0.9635 | 0.3775 | 0.6315 |
| N0LC1 | 0.7505 | 0.2875 | −0.1615 | 0.3605 | 0.8885 | 1.4155 | 1.8215 | 1.2875 | 0.3995 | 1.0825 | −0.1645 | 1.6375 | 0.2115 | 0.9675 | 0.6425 |
| NotFound | −0.4525 | −0.4145 | −0.1395 | −0.2805 | −0.8665 | −0.0315 | −0.6355 | −0.6235 | −0.2365 | −0.7785 | 0.1295 | 0.2725 | 0.4485 | −0.4155 | 0.4185 |
| NotFound | −0.467 | 0.019 | 0.759 | 0.795 | −0.352 | −0.888 | 0.018 | 0.569 | 1.101 | 0.042 | 1.119 | −0.1 | 1.003 | 0.261 | 0.244 |
| NotFound | −0.793 | −2.085 | −0.087 | −0.095 | −2.377 | 0.14 | −0.8 | −1.851 | −3.231 | −0.386 | −0.52 | 0.362 | −1.935 | −0.059 | −0.722 |
| NotFound | 0.467 | 0.067 | 1.248 | 1.399 | −0.094 | −0.06 | −0.318 | −0.371 | 1.17 | 0.07 | 0.437 | −0.139 | 0.353 | 0.104 | 0.552 |
| NotFound | 1.2945 | −0.6545 | 1.6465 | 1.6795 | −0.5355 | 0.1595 | −0.0915 | 1.1315 | 0.8025 | −0.6375 | 0.9905 | 1.6085 | 2.7345 | 0.5145 | 1.0025 |
| NotFound | −0.199 | 3.879 | 0.593 | 4.087 | 1.726 | 2.925 | 2.827 | −0.168 | 3.332 | 1.763 | 4.134 | −0.704 | −1.1 | −0.852 | −1.147 |
| NotFound | −1.2875 | 2.8405 | −1.4995 | 0.9595 | 0.6025 | −0.3015 | −0.3475 | 1.0385 | −2.1895 | −1.2035 | 1.0145 | 0.8855 | −0.3876 | −0.1505 | 0.6985 |
| NotFound | −1.058 | −0.805 | 0.076 | 0.081 | 0.932 | 0.365 | 0.086 | 0.021 | 0.588 | 1.467 | 0.302 | 0.724 | 0.227 | −0.012 | 1.929 |
| NotFound | 0.8045 | 0.7055 | 0.3265 | −0.5355 | −0.0575 | −0.1475 | 0.1755 | 0.7935 | −0.5745 | −0.6475 | 0.0765 | 0.7575 | 1.0575 | 1.1615 | −0.0595 |
| NotFound | −0.3855 | 0.3025 | 1.3885 | 1.3065 | −1.5685 | 0.0185 | 0.7975 | 0.4545 | 1.0535 | −0.5535 | 1.5965 | −0.8005 | 1.4485 | 0.2045 | −0.0025 |
| NotFound | −0.8895 | 0.0685 | 0.1255 | −0.0155 | −0.2655 | 0.1885 | −1.1345 | −0.3675 | 0.2965 | −0.1035 | 0.0035 | 0.4285 | −0.2615 | 0.1055 | 1.1465 |
| NotFound | 0.1825 | 0.1515 | 0.6225 | 0.3995 | 1.0185 | 0.4275 | −0.2105 | 0.0035 | 0.1345 | 0.2195 | 0.0105 | 0.9585 | 2.2175 | −0.1195 | 0.1475 |
| NotFound | −1.508 | −0.555 | −1.388 | 0.038 | −1.149 | −0.229 | −0.805 | −0.016 | 0.15 | −0.932 | 0.372 | −1.212 | −1.544 | −0.871 | −2.509 |
| NotFound | 0.387 | 0.132 | −0.341 | −0.257 | −0.019 | 0.23 | −0.612 | −0.383 | −0.155 | −0.306 | 0.226 | −0.913 | −0.318 | −0.194 | −0.108 |
| NotFound | −1.556 | −2.213 | −0.99 | −1.504 | −1.208 | −1.201 | −1.746 | 1.482 | −1.391 | −2.363 | −1.903 | −2.415 | −1.194 | −1.845 | −1.713 |
| NotFound | −1.9335 | −2.2025 | −1.0105 | −2.3455 | −1.7925 | −1.0225 | −0.8335 | −1.5795 | −1.4765 | −0.9695 | −2.0055 | −1.9175 | −1.0055 | −1.7625 | −2.0526 |
| NotFound | −1.261 | −0.44 | −0.442 | −0.848 | −0.582 | −0.979 | −0.349 | −0.765 | 0.263 | −0.123 | 1.421 | −0.645 | 0.445 | −0.201 | −0.836 |
| NotFound | −0.243 | 0.544 | 0.338 | 0.913 | −0.227 | 0.185 | 0.302 | 0.624 | 0.371 | −0.154 | 1.198 | 0.372 | −0.166 | −0.323 | 0.845 |
| NotFound | 0.5785 | −0.6335 | −0.3505 | −0.4195 | 0.6035 | 0.0525 | −0.7435 | −0.3165 | 0.5295 | −0.9195 | −0.8515 | −0.0555 | −0.1155 | 1.0625 | 0.5045 |
| NotFound | −3.0805 | −2.5495 | 0.5035 | −0.1895 | 0.4215 | 0.0075 | −1.2605 | −1.6295 | −1.5415 | −2.0545 | −1.9716 | −2.3555 | −0.4515 | −0.7595 | −0.0075 |
| NotFound | −1.247 | −0.83 | −1.11 | −0.841 | −1.004 | 0.338 | −1.279 | −1.003 | 0.297 | −0.728 | −0.833 | −1.876 | −0.665 | −0.922 | −0.875 |
| NotFound | 0.6615 | 1.0915 | 2.3775 | 1.8505 | 2.8535 | 0.9655 | 0.7185 | 0.4905 | 0.7895 | 0.7525 | 1.8715 | 1.1075 | 0.9735 | 1.2445 | 0.5575 |
| NotFound | −0.762 | −2.131 | −1.476 | −1.743 | −1.509 | −1.555 | −1.683 | −1.756 | −1.384 | −1.913 | −1.927 | −1.35 | −1.517 | −1.232 | −1.172 |
| NotFound | −0.362 | 1.889 | −0.652 | −0.079 | 0.363 | −0.456 | −0.205 | 0.032 | −0.856 | −0.622 | 0.481 | 0.391 | 0.583 | 0.409 | 0.773 |
| NotFound | 1.3785 | −0.5115 | 0.9745 | 1.5855 | 1.7385 | 1.9335 | 0.2725 | 0.3135 | 0.5245 | 0.0215 | 1.1795 | 1.9675 | 0.4755 | 1.1425 | 1.2485 |
| NotFound | 0.423 | 0.96 | 0.949 | 0.91 | 1.129 | 0.177 | 0.795 | 0.807 | 0.476 | 0.6 | 0.855 | 0.377 | −0.401 | 0.791 | −0.058 |
| NotFound | −0.1415 | 1.8455 | −1.0025 | −0.0185 | 0.1965 | 0.1915 | −0.6935 | −0.0155 | −0.8375 | −0.7785 | 0.8715 | 0.3835 | 0.2165 | 0.4505 | 0.4495 |
| NotFound | 3.323 | −0.998 | 2.809 | 2.991 | −0.114 | 0.091 | 3.884 | 1.863 | −0.396 | −0.423 | −0.689 | 0.205 | 1.978 | −0.738 | 3.118 |
| NotFound | −0.274 | −0.101 | −0.693 | −0.041 | −0.705 | −0.241 | −0.375 | −0.602 | −0.328 | 0.621 | −1.157 | −0.881 | −0.878 | −0.531 | −0.499 |
| NotFound | −0.4755 | 0.2246 | 0.5685 | 0.4245 | −0.3385 | −0.1175 | −0.0415 | 0.1755 | 1.0355 | 0.0165 | 1.3825 | −0.8525 | 1.1205 | 0.8345 | 0.4085 |
| NotFound | −0.4435 | 1.7925 | 0.0115 | 0.1845 | 0.0055 | 0.3965 | −0.6365 | −0.0415 | −0.4295 | −0.8995 | 0.7625 | −0.0055 | −0.1705 | 0.3005 | 0.0225 |
| NotFound | −0.0485 | 2.5635 | −0.6805 | −0.6215 | −0.7305 | −0.9045 | −0.1375 | 0.1515 | −0.7675 | −0.8845 | 0.6935 | −0.0235 | −0.4545 | 0.0845 | −1.1425 |
| NotFound | −0.2255 | 1.8955 | −0.6235 | 0.0855 | 0.5415 | 0.1785 | −0.3205 | −0.1315 | −0.9145 | −0.8085 | 0.6355 | 0.7225 | 0.7705 | 0.8905 | 0.8245 |
| NotFound | −0.2505 | −2.3805 | −0.8995 | −1.1925 | −2.7315 | 0.3935 | 0.4225 | −0.5295 | 0.5845 | −0.6555 | −0.0415 | 0.6985 | −0.3295 | 0.8365 | −1.7015 |
| NotFound | −0.297 | 2.22 | 0.15 | −0.269 | 0.519 | 0.28 | −0.307 | 0.081 | 0.093 | −0.193 | 0.052 | −0.008 | 0.032 | 0.805 | −0.199 |
| NotFound | −1.4235 | 1.4895 | −2.0045 | −0.4475 | 0.3005 | 0.0295 | −0.5185 | 0.0075 | −0.8045 | −0.9195 | −0.1645 | 0.6475 | 0.3645 | 0.7315 | 0.9235 |
| NotFound | 0.6935 | 0.1055 | 0.1876 | 0.1015 | −0.2415 | 0.1125 | −0.1815 | 0.0045 | −0.6555 | −0.3866 | −0.2765 | 0.0735 | 0.5765 | 0.1575 | 0.1805 |
| NotFound | 1.5455 | 3.1825 | −0.5755 | −0.0425 | 0.3995 | 0.0105 | −0.8375 | −0.1895 | −0.3185 | −0.1855 | 0.5685 | 0.0035 | 0.4015 | 1.9565 | −0.3425 |
| NotFound | 0.3265 | −0.3785 | −0.3715 | −0.8735 | −1.6225 | −1.1795 | −1.5765 | −0.3195 | 0.0635 | 0.2305 | −0.2125 | −0.2575 | −0.4475 | −1.2635 | −0.7825 |
| NotFound | −0.52 | 0.058 | 4.384 | 0.285 | 1.046 | 0.708 | 1.59 | −0.704 | 2.668 | 1.988 | 4.076 | 1.154 | 1.901 | 1.62 | 3.095 |
| NotFound | −0.182 | −0.771 | 0.02 | 0.242 | 0.205 | 0.119 | −0.195 | −0.077 | 0.581 | 0.122 | 0.054 | −0.198 | −0.119 | −0.27 | 0.096 |
| NotFound | −0.085 | 0.205 | 1.153 | 0.66 | −0.557 | 0.081 | 0.408 | 0.993 | −0.454 | −0.204 | 2.711 | −0.107 | 0.663 | 1.01 | 0.781 |
| NotFound | 0.0145 | 1.9765 | −0.5685 | 0.2315 | 0.6145 | 0.4315 | −0.3845 | −0.0555 | −0.4925 | −0.7385 | 0.8075 | 0.3335 | 0.2825 | 0.8025 | 0.5245 |
| NotFound | 0.9185 | 0.3426 | 0.2595 | 0.8515 | 1.6415 | 0.5335 | 1.6695 | 2.0065 | 0.1335 | 1.0635 | 0.3355 | 1.5505 | 0.7945 | 1.7855 | 0.7785 |
| NotFound | 0.092 | 0.295 | 0.269 | 0.152 | −0.122 | −0.288 | −0.318 | −0.165 | −0.075 | 0.074 | 0.187 | −0.083 | 0.55 | −0.127 | −0.201 |
| NotFound | −0.4855 | −0.1755 | −0.3495 | 0.1485 | 0.1705 | −8.4315 | 0.2865 | 0.1815 | 0.2825 | 9.8325 | 0.3715 | 0.2115 | 0.2325 | −0.1225 | −0.0095 |
| NotFound | 0.5695 | 0.5395 | 0.5345 | −0.5455 | 1.1155 | −1.0005 | −1.2485 | −0.4825 | 0.4245 | −0.0445 | 1.1935 | −0.0925 | −0.2215 | 0.2385 | 0.8845 |
| NotFound | 0.6095 | −0.5276 | 1.0815 | 0.0685 | 0.1325 | 0.4445 | 0.4785 | 0.2235 | −0.6715 | −0.1525 | 0.3245 | 0.0115 | −0.9325 | 0.3985 | −0.2785 |
| NotFound | 0.0315 | −0.4436 | 0.6215 | 0.1515 | −0.3975 | −0.0715 | −0.5815 | −0.2535 | −0.7785 | −0.6025 | −0.8155 | −0.1955 | −0.7985 | 0.1025 | −0.1755 |
| NotFound | 0.26 | −0.433 | −0.932 | −0.255 | 0.181 | −0.233 | −0.083 | −0.116 | −1.328 | −0.416 | 0.378 | 0.62 | 0.067 | −0.404 | −0.537 |
| NotFound | −1.401 | −1.151 | −1.659 | −1.481 | −0.814 | −1.081 | −1.329 | −1.185 | −0.79 | −1.289 | −0.875 | −0.787 | −0.91 | 1.366 | −0.842 |
| NotFound | −0.8805 | −1.1995 | 0.1535 | −0.0895 | −1.3675 | −0.0095 | −0.8015 | −1.7105 | −2.7765 | −0.3315 | −0.3465 | 0.1275 | −1.6765 | −0.0095 | −0.7565 |
| NotFound | −0.8825 | −0.7365 | −0.3035 | −0.0055 | −0.5055 | −0.0255 | −1.1825 | −1.4155 | −1.1725 | −1.6775 | −0.9365 | −2.1935 | −0.8095 | −0.8565 | −0.9985 |
| NotFound | −1.3045 | −0.6205 | −2.1225 | −1.8095 | −1.3815 | −0.1845 | −0.5845 | −1.0485 | −0.6955 | −0.8335 | −1.7475 | −1.2165 | −0.9555 | −0.5135 | −1.5205 |
| NotFound | −0.1545 | 0.3885 | 1.2875 | 0.2945 | −0.7235 | 0.0585 | 1.6735 | −0.6385 | −0.5075 | 0.2775 | 0.5005 | 0.5145 | −0.0805 | 0.0765 | 1.6605 |
| NotFound | 1.133 | 1.717 | 3.035 | 0.642 | 1.233 | 1.093 | 0.228 | 0.431 | 0.684 | 0.649 | 1.748 | 0.173 | −1.093 | −0.358 | −0.01 |
| NotFound | −0.292 | −1.195 | −1.312 | −0.381 | −0.812 | 1.139 | −0.247 | −0.663 | −0.084 | −0.909 | −0.841 | −0.916 | −0.325 | −0.369 | −0.327 |
| NotFound | −0.1915 | 0.0335 | 0.4575 | 0.0935 | −0.0935 | −0.2585 | 0.0055 | 0.1995 | 0.8045 | −0.0466 | 0.2405 | 0.1045 | 0.2375 | −0.0465 | 0.0555 |
| NotFound | −1.655 | −0.752 | −1.558 | 0.248 | −1.177 | −0.002 | −1.111 | −2.158 | −0.489 | −0.244 | 0.002 | −1.289 | 1.326 | −1.004 | −0.834 |
| NotFound | 0.012 | 0.186 | −0.542 | −0.538 | 0.133 | 0.416 | 0.283 | 0.02 | 0.798 | 0.266 | 0.101 | −0.766 | 0.373 | 0.624 | 0.518 |
| NotFound | −0.5335 | −0.6345 | 0.5055 | −0.7245 | 0.2705 | 0.4675 | −0.8455 | −0.5425 | −0.1275 | −0.6265 | 0.3285 | −0.8145 | −0.7035 | −0.3605 | −0.3685 |
| NotFound | −0.032 | 0.461 | −0.343 | 0.243 | 0.722 | 0.181 | −0.195 | −0.103 | 0.066 | 0.049 | −0.342 | −0.185 | 0.07 | 0.168 | 0.161 |
| NotFound | 0.063 | 2.705 | −1.048 | −0.931 | −0.819 | −1.167 | −0.258 | 0.145 | −1.347 | −0.722 | 1.012 | −0.01 | −0.641 | 0.311 | −1.213 |
| NotFound | −0.7765 | −1.5835 | 0.2355 | −0.2335 | 0.1965 | −0.8505 | −0.6685 | −0.3415 | 0.1215 | −1.5935 | −0.4645 | 1.0685 | −0.3685 | −0.6805 | −0.1615 |
| NotFound | 0.248 | −0.771 | −0.828 | −0.518 | −0.4 | −0.354 | −1.056 | −0.544 | −0.656 | −1.115 | −0.391 | −1.396 | −0.188 | −0.139 | −0.485 |
| NotFound | 0.0265 | −0.1735 | 0.1045 | 0.8825 | 1.0215 | 1.0515 | −0.2225 | 0.1585 | −0.1825 | −0.0255 | −0.1455 | 1.3705 | 2.7455 | 1.0005 | 0.3735 |
| NotFound | −0.4505 | −0.2455 | −0.1455 | −0.0955 | 0.0095 | −0.4595 | 0.1935 | 0.1655 | −0.0115 | 0.3135 | 0.0615 | 0.3515 | 9.9975 | −0.2685 | 0.2635 |
| NotFound | −0.314 | 1.514 | −1.024 | −0.4 | 0.279 | −0.137 | −0.433 | −0.085 | −0.638 | −0.743 | 0.494 | 0.135 | 0.039 | 0.213 | 0.213 |
| NotFound | 0.5855 | −0.6245 | 0.1415 | 0.3835 | 1.4335 | 0.2065 | 0.1835 | 0.4505 | 0.2725 | −0.4245 | 0.0235 | 1.3445 | 0.7485 | 1.0915 | 0.6993 |
| NotFound | −1.403 | −0.734 | −2.017 | 0.364 | −2.764 | −0.141 | −1.297 | −1.552 | −2.413 | −2.497 | −0.873 | −2.688 | −0.639 | −1.406 | −2.712 |
| NotFound | −0.34 | −0.537 | −0.559 | −0.635 | −0.777 | −0.197 | −0.178 | −0.342 | −0.941 | 0.036 | 0.158 | −0.28 | −0.766 | 0.05 | −0.963 |
| NPDC1 | 0.1275 | −0.4615 | −1.0005 | −0.2435 | −0.5525 | −0.1215 | 0.0405 | 0.2575 | −0.3635 | −0.5025 | −0.6505 | 0.0795 | 0.6575 | −0.6735 | 0.3735 |
| NPEPPS | −0.923 | −1.122 | −1.21 | −0.088 | 0.208 | −0.328 | −0.172 | −0.317 | −0.787 | −1.049 | −0.54 | 0.55 | −0.602 | 0.193 | −0.634 |
| NPY | 1.662 | 1.129 | 0.802 | 0.083 | 1.218 | 0.634 | 1.182 | 1.064 | 1.304 | −0.911 | 0.159 | 1.541 | 0.754 | 0.814 | 1.464 |
| NQO3A2 | −0.506 | −2.211 | 0.306 | 0.332 | −0.349 | −1.472 | −2.248 | −1.006 | −0.853 | −2.293 | 1.882 | −1.448 | 0.556 | 0.278 | −0.648 |
| NRAS | −0.065 | −0.043 | −2.025 | 1.005 | −3.73 | −0.486 | −0.565 | −2.576 | −1.115 | −1.667 | −1.539 | −2.131 | −2.56 | −2.677 | −3.997 |
| NRIP1 | 1.0815 | −0.9595 | −1.4525 | 0.4385 | −0.7775 | −0.1775 | −0.8385 | −1.0305 | −0.9025 | −0.6485 | −0.1535 | −1.4015 | −0.4205 | −0.4445 | 1.1335 |
| NTN4 | 0.4515 | 0.4175 | 1.3085 | 0.9435 | 2.3815 | 1.4645 | 0.4425 | 1.2025 | 0.6355 | 0.5505 | 0.5295 | 1.3575 | 0.6595 | 1.1645 | 0.9116 |
| NUCB2 | −0.5365 | −0.3725 | −0.1215 | −0.5305 | −0.1525 | −0.4205 | 0.1335 | −0.3515 | −0.8535 | −0.4485 | −0.8595 | −1.2855 | −0.5675 | 0.3965 | 0.2735 |
| NUDT5 | −0.755 | −0.061 | −0.477 | 0.377 | −0.15 | −0.703 | 1.186 | 0.35 | 0.009 | 0.608 | 1.428 | 0.934 | 0.492 | 0.624 | 1.377 |
| NUDT6 | −0.223 | 0.068 | −0.359 | −0.04 | −0.209 | −0.071 | 0.758 | −0.27 | −0.2 | −0.08 | 0.806 | −0.26 | −0.103 | −0.345 | −0.061 |
| OCIAD2 | 1.0315 | 1.8215 | 2.3085 | 2.1815 | 3.1615 | 1.0205 | 0.8255 | 0.7035 | 1.4395 | 1.1585 | 1.4475 | 0.8045 | 0.2495 | 1.7535 | 2.3275 |
| OFD1 | 0.6055 | 3.0175 | 1.9075 | 2.5515 | 1.3415 | 1.2995 | 1.3585 | −2.2835 | −0.2915 | −1.0595 | −0.2485 | 0.8305 | −0.0905 | −0.7005 | 1.0255 |
| OGFRL1 | −0.26 | 0 | −0.302 | −0.325 | −0.69 | −0.571 | −0.775 | −0.567 | −0.267 | −0.323 | −0.345 | −0.569 | −0.351 | −0.958 | −0.664 |
| OPLAH | 1.4025 | 1.0325 | 0.2555 | 0.2815 | −0.7385 | −0.0645 | 0.1935 | 0.4825 | 0.3895 | 0.0685 | 0.3155 | 0.9255 | 0.9655 | 0.0445 | −0.0655 |
| ORMDL3 | −2.346 | −0.831 | −2.778 | 0.962 | −2.293 | 1.505 | 0.465 | 0.236 | −0.996 | −2.141 | −2.645 | 1.085 | 1.531 | −2.084 | −1.208 |
| OSBPL3 | 0.11 | −0.123 | 0.812 | −0.051 | −0.153 | 0.574 | −0.068 | −0.094 | 0.2 | 0.018 | −0.564 | 0.559 | 0.032 | 0.579 | 0.042 |
| OSR2 | −0.1265 | 0.0975 | 0.8055 | 0.9885 | 0.2825 | 0.2745 | 0.0865 | 0.5465 | 0.4365 | 0.0605 | 0.4795 | −0.4595 | 1.0855 | −0.0495 | 0.6065 |
| OSTF1 | 1.859 | −3.002 | 0.249 | −0.580 | 0.441 | −0.191 | −1.055 | −1.539 | −1.731 | −1.915 | −2.77 | −0.983 | 0.191 | 0.886 | −0.423 |
| P2RX1 | −1.1705 | −0.7025 | 0.3705 | 0.0935 | −1.1365 | −1.0105 | −0.9225 | −0.2975 | 0.3225 | −0.2005 | 0.1995 | −1.2375 | 0.8825 | 0.1615 | 0.4335 |
| P2RY2 | −0.069 | 1.746 | 1.248 | 0.666 | 1.082 | 1.421 | −0.053 | −0.098 | 0.35 | −0.881 | 2.057 | 1.722 | 1.05 | −0.028 | 0.396 |
| PACAP | −0.403 | 1.24 | 1.005 | 0.822 | 0.307 | 0.021 | −0.259 | 0.528 | −0.317 | −0.284 | 0.224 | 0.294 | 0.621 | −0.071 | −0.04 |
| PADI2 | −0.0215 | −1.5235 | 1.1475 | −0.4445 | 1.2795 | −0.4925 | −0.5155 | −0.1475 | 0.3445 | 0.2005 | −0.0025 | −1.2425 | 0.4445 | −0.9855 | −0.5135 |
| PAPD5 | −0.208 | 1.4 | 0.062 | −0.108 | −0.782 | −0.8 | −0.052 | 0.01 | 0.269 | −0.004 | −0.018 | −0.537 | −0.131 | −0.925 | −0.297 |
| PAPSS2 | −0.244 | 0.452 | 0.431 | −0.031 | 0.499 | 0.389 | 0.657 | 0.085 | 0.426 | 0.264 | 0.808 | 0.966 | 0.045 | 0.589 | 0.688 |
| PARD8A | 0.818 | 0.504 | −0.303 | −0.103 | 0.21 | 0.138 | −0.511 | −0.123 | −0.12 | −0.486 | 0.079 | −0.008 | 0.289 | 0.347 | 0.081 |
| PARP1 | −0.03 | 0.546 | 0.131 | −0.577 | −0.161 | −0.289 | 0.643 | −0.011 | 0.034 | 0.389 | 0.203 | 0.049 | −0.279 | 0 | −0.112 |
| PARP9 | 0.5265 | 0.1915 | −0.1165 | −0.2525 | 0.1325 | −0.3795 | −1.0695 | −0.2115 | −0.3635 | −0.8945 | −0.2905 | 0.1505 | −0.2875 | −0.5405 | −1.6176 |
| PAXIP1L | 0.0115 | −0.0235 | −0.1745 | 0.6745 | 2.8505 | 1.0895 | 0.7435 | 0.9305 | −0.6675 | 1.1565 | 1.3285 | 0.5985 | 0.1745 | 1.0545 | 1.8695 |
| PBX3 | −0.5945 | 0.8825 | −1.3775 | −0.4065 | 2.4585 | 0.1485 | −0.2255 | 1.9125 | 0.2205 | −0.3905 | −1.0755 | 0.7395 | −0.7375 | 1.7295 | −0.4195 |
| PCDHB11 | −1.621 | 1.318 | 1.417 | −0.745 | 3.53 | 2.728 | 1.768 | 1.919 | −1.054 | 3.208 | 3.222 | 2.411 | 1.474 | 3.05 | −0.918 |
| PCDHB16 | 0.72 | −1.316 | −0.453 | −0.125 | −0.878 | 0.155 | 0.807 | −0.04 | −0.432 | −0.129 | 0.774 | −0.225 | −1.05 | 0.254 | −0.093 |
| PCDHB2 | 1.7145 | −0.2055 | 0.4945 | 0.9285 | 0.2815 | 1.6265 | 0.7925 | −0.3525 | 0.5425 | 0.3475 | 0.2765 | 0.7805 | 0.1155 | 0.1535 | −0.0855 |
| PCM1 | −0.0275 | −0.1225 | 0.4515 | 0.8705 | −0.2165 | 0.0505 | −0.2215 | 0.1405 | 0.2046 | 0.0085 | 1.4085 | −0.0335 | 0.6795 | −0.2705 | 0.5235 |
| PCNA | 0.243 | 0.318 | 1.187 | 0.941 | 0.101 | −0.048 | 1.035 | 0.165 | 0.897 | −0.029 | 0.184 | 0.385 | 1.213 | 0.509 | 1.695 |
| PDCD1 | −0.5925 | −0.2805 | −0.9015 | −0.2475 | 1.0345 | 0.1145 | −1.1665 | −1.0005 | −1.5065 | −0.6515 | −1.2775 | −1.6455 | −1.4195 | −0.7505 | −0.7535 |
| PDCD10 | 1.186 | 0.445 | 1.07 | 0.635 | 1.788 | 0.947 | 1.19 | 1.527 | 0.518 | 0.024 | 0.28 | 0.209 | 1.29 | −0.081 | 1.305 |
| PDCD4 | −0.1475 | −0.5435 | −0.1865 | −0.1955 | 0.4085 | 1.1355 | −0.1805 | −1.0115 | 0.2055 | −0.7125 | −0.3195 | 0.2125 | −0.4775 | 0.8836 | 0.2135 |
| PDCD5 | −0.0405 | −1.0675 | −1.0195 | −1.0565 | −1.2085 | −0.1825 | −0.2785 | 0.6175 | −0.0895 | −0.8775 | 0.1875 | 1.5055 | 1.0345 | −0.2135 | 0.2565 |
| PDCD8 | 0.7095 | 0.8725 | 0.8295 | 0.8755 | 1.5775 | 0.4845 | 0.9595 | 0.2925 | 0.2285 | 0.4715 | 0.1815 | 0.8755 | 0.6785 | 2.0255 | 1.0655 |
| PDE4DIP | −1.9135 | −0.7265 | −2.4785 | −2.0695 | −3.2025 | −0.7875 | −0.1685 | −1.2775 | −0.9505 | −1.0245 | −1.2585 | −1.6945 | −1.3025 | −1.2095 | −0.2225 |
| PDHA1 | 1.635 | 0.86 | 0.23 | 0.627 | 2.575 | 0.039 | 1.974 | 1.955 | −0.197 | 1.283 | 1.074 | 3.541 | 1.718 | 0.428 | −1.17 |
| PECI | −0.2225 | 0.1215 | 0.4795 | −0.7405 | −1.0705 | −0.5505 | −0.8415 | −0.6515 | −0.0525 | 0.3465 | −0.5735 | −1.1795 | −1.7325 | 0.4855 | −0.5615 |
| PEG10 | 1.3325 | 0.6215 | −0.6285 | 1.1885 | −0.3885 | 0.2665 | 1.9455 | 1.3695 | 0.9145 | 0.3965 | 0.5085 | 0.3085 | 0.3395 | 1.7975 | 0.6025 |
| PER2 | −0.121 | −0.425 | −0.964 | 0.543 | 0.124 | −0.052 | 0.38 | 0.456 | −0.378 | −0.184 | 0.289 | −0.152 | −0.469 | 0.304 | −0.073 |
| PERP | −1.0545 | 0.3205 | 0.2105 | −0.6745 | −0.0895 | −0.2195 | 0.0105 | 0.0025 | 0.1475 | 0.6125 | −0.0375 | 0.9475 | −0.1145 | 0.2335 | 0.4175 |
| PEX1 | 0.848 | 0.762 | 0.82 | 0.474 | 1.105 | 0.343 | 0.353 | 0.32 | 0.545 | −0.113 | 0.393 | 0.459 | 0.703 | 1.291 | 0.257 |
| PFAAP5 | −0.3875 | 0.0095 | 0.8055 | −0.3735 | 0.4825 | −0.2485 | 1.0015 | 1.4185 | −0.8275 | −1.3205 | −0.3275 | −0.8135 | −0.8655 | 0.7795 | −0.5815 |
| PFDN2 | −1.025 | −1.38 | −1.833 | −1.466 | −1.107 | −0.01 | 0.436 | 0.346 | −0.157 | −1.592 | −0.87 | −0.893 | −0.392 | 0.674 | −0.544 |
| PGM1 | −0.1495 | −0.1395 | −0.2205 | −0.1565 | −0.2795 | 0.5415 | 0.0095 | 0.6885 | −0.1695 | 0.2195 | −0.5845 | −0.1905 | 0.3275 | −0.1265 | 0.2715 |
| PGR1 | 1.2065 | 1.3835 | 2.0115 | 1.3925 | 1.6235 | 2.1955 | 0.7855 | 0.7085 | 1.0685 | 0.4465 | 1.8375 | 2.5695 | −0.8745 | 1.1445 | 1.4425 |
| PHAX | 0.751 | −0.205 | 0.725 | 1.294 | 2.99 | 1.388 | 0.202 | 0.684 | −0.48 | −1.028 | −0.84 | 2.057 | 0.391 | 1.797 | 0.558 |
| PHGDH | −2.2835 | −2.7635 | −0.0625 | −3.3415 | −2.0755 | −0.5335 | −2.1785 | −1.6285 | −0.8995 | −2.5985 | −1.0655 | −1.3165 | −1.4435 | −2.9645 | −2.8275 |
| PHYH | 1.0065 | −0.5995 | −0.9825 | −0.5565 | −0.7775 | −0.1165 | −0.9795 | −0.4625 | −1.3225 | −0.7775 | 0.1575 | −0.3915 | −0.7145 | −0.1565 | −0.2755 |
| PI85PA | −0.7425 | −0.1575 | 0.5295 | 0.4105 | −0.4315 | 0.0755 | −0.5565 | 0.1945 | 1.0205 | −0.0435 | 0.7716 | −1.0045 | 0.4055 | 0.7535 | 0.4825 |
| PIGT | −1.077 | −0.018 | 0.344 | −0.265 | −0.29 | −0.236 | −0.518 | −0.301 | −0.005 | −0.353 | 0.416 | −0.818 | −0.475 | −0.61 | −1.196 |
| PIM2 | 0.5516 | −1.7185 | −2.0765 | −0.8775 | −1.2915 | 0.0185 | 0.3905 | 0.5685 | −0.4055 | −0.6475 | −0.7955 | −0.0185 | −0.1415 | 0.6505 | 0.8415 |
| PISD | −1.508 | −2.875 | −2.184 | −2.094 | −1.692 | −0.842 | −0.247 | −0.59 | −0.729 | −0.839 | −2.122 | 0.457 | 2.533 | −1.309 | −1.256 |
| PITPNC1 | 1.0265 | −0.1695 | 1.2675 | 0.9655 | 0.7635 | 1.0815 | −0.4605 | −0.0225 | 0.0635 | 0.5515 | 0.6935 | 0.0485 | 0.1035 | 0.1995 | 0.1415 |
| PKIB | −1.293 | −0.082 | −0.68 | −0.358 | −0.649 | −0.919 | −1.108 | −0.484 | 0.077 | −0.403 | −0.272 | −1.03 | −0.669 | −0.618 | −0.111 |
| PKMYT1 | −0.526 | −0.978 | 0.762 | 1.455 | 1.235 | 0.485 | −0.392 | −0.379 | 0.584 | −0.385 | 1.067 | 0.943 | 0.582 | 1.627 | 2.195 |
| PLA2G4B | 0.5905 | 1.5795 | 0.0045 | 1.6325 | −0.3385 | 0.8395 | 0.1785 | −0.3345 | 1.3205 | 1.6045 | −0.1075 | −1.2805 | 0.0975 | −0.6345 | −0.2705 |
| PLAC8 | 0.026 | −0.093 | 0.85 | −0.063 | −0.185 | 0.61 | 0.297 | −0.926 | −0.21 | 0.073 | 0.889 | −0.721 | −0.29 | 0.29 | 0.04 |
| PLCB1 | −0.0505 | −1.2785 | 0.2985 | −0.5445 | −0.8595 | −0.6595 | −0.2405 | 0.4915 | 0.0085 | −0.2365 | −0.8525 | 0.4145 | 0.4075 | −0.0725 | 1.4005 |
| PLCXD1 | 1.608 | 0.141 | −0.187 | −0.427 | 0.516 | 1.417 | 1.14 | 0.913 | 0.8 | 0.29 | −0.13 | 0.24 | 1.688 | 1.186 | 1.621 |
| PLEK2 | 0.0265 | 0.3445 | 0.1875 | 0.2535 | 0.4785 | 0.1235 | 1.9035 | 1.2445 | 0.0445 | 0.7825 | 0.7525 | 1.2475 | 0.6975 | 1.0545 | 0.8945 |
| PLO0 | −0.3815 | −0.4005 | −0.8545 | −0.4925 | −0.5935 | 0.3535 | 0.4055 | 0.0965 | 0.0065 | −0.0765 | −0.5085 | −0.1115 | −0.2605 | −0.1775 | −0.1805 |
| PNAS-4 | 0.1755 | 0.2825 | 1.4365 | 0.6325 | 2.9845 | 1.7635 | 0.7925 | 0.7185 | 1.8555 | 0.1955 | 2.2005 | 2.4445 | 2.4195 | 0.3585 | 0.9305 |
| PNLIPRP2 | −0.5875 | −1.3385 | −0.1915 | 0.2335 | 1.4165 | −0.0185 | 0.3585 | 0.3745 | −0.5185 | −0.5525 | −0.0145 | 0.2935 | 0.0985 | 0.0935 | 0.3085 |
| PNMA3 | 0.4015 | 0.7945 | 1.2605 | 0.6415 | 1.3845 | 0.8185 | 0.5095 | 0.0725 | 0.5825 | 0.2745 | 0.9585 | 0.3815 | −0.1645 | 0.2705 | −0.1395 |
| POLB | −0.755 | −0.383 | 0.602 | 0.292 | 0.383 | −0.052 | −0.543 | 0.055 | 0.488 | 0.571 | 0.156 | 0.461 | 0.205 | 0.143 | 0.837 |
| POLR2F | −0.0075 | −0.8195 | 0.4845 | 0.5845 | 0.0955 | 1.9115 | 0.5275 | −0.0915 | 0.0955 | 0.5895 | 1.8125 | −0.8385 | −0.2595 | −0.3255 | 0.1255 |
| POLR2H | 0.0035 | −0.1515 | 0.3385 | 0.1355 | 1.2655 | 0.6715 | 0.4025 | 0.3235 | 0.0285 | −0.6725 | −0.0715 | −0.4035 | 1.4575 | −0.2075 | −0.1785 |
| POMC | 0.542 | 0.685 | 0.414 | 1.109 | 1.091 | 0.679 | 1.024 | 0.096 | 0.323 | −0.037 | 0.101 | −0.128 | 0.034 | 0.413 | 0.298 |
| POP4 | −1.524 | −1.182 | 0.637 | −0.707 | −1.891 | −0.015 | −0.871 | −0.925 | −0.122 | 0.026 | −0.932 | −1.215 | −0.73 | −1.023 | −1.375 |
| POP7 | −0.01 | −0.667 | −0.43 | −0.44 | 0.799 | −0.019 | 0.356 | 1.029 | 0.454 | 0.447 | 0.243 | 0.075 | 0.214 | −0.123 | 0.247 |
| PP1057 | −0.8005 | −0.2545 | 0.8765 | −0.5405 | 0.1105 | 0.0705 | −0.1765 | 0.0575 | −0.9735 | −0.0345 | 0.4655 | 0.4525 | −0.4185 | 0.0465 | 0.1165 |
| PPFIA1 | 1.278 | −0.193 | 0.469 | 0.438 | 0.549 | −0.13 | −0.638 | 0.114 | 0.179 | −0.364 | −0.631 | 0.609 | 0.029 | 1.087 | 0.319 |
| PPFIBP2 | −0.8575 | 0.0185 | −0.4005 | 0.1705 | −0.4875 | 0.1095 | −0.0425 | −0.2475 | 0.2455 | −1.1405 | −0.5355 | 0.0645 | 0.2905 | −0.0425 | −0.1255 |
| PPGB | 0.4535 | −0.3225 | 0.4435 | 0.6095 | 0.5795 | 0.6745 | −1.2055 | −0.2065 | 0.5785 | −0.3775 | 0.7025 | 0.9015 | 0.8915 | 1.5575 | −0.1905 |
| PPHLN1 | 0.2365 | −0.1425 | 0.4755 | 0.5825 | −0.2515 | 0.1755 | 0.4565 | 0.4875 | 0.1285 | −0.0715 | 1.1215 | 0.5295 | 0.8125 | 0.3945 | −0.3945 |
| PPIF | 0.2955 | −0.1045 | −0.5135 | −0.4015 | −0.2365 | −0.5995 | 0.0845 | −0.4005 | −0.3305 | −0.6325 | 0.1415 | −0.9795 | −0.1475 | 0.2685 | 0.2485 |
| PPIL1 | 0.7365 | 1.4045 | 0.7995 | −0.3475 | 0.3155 | 0.3285 | 1.8185 | −1.1205 | −0.8125 | −1.0835 | −0.3225 | 0.4155 | 0.0045 | 0.4475 | 1.1325 |
| PPOX | 0.03 | 0.088 | −1.235 | −2.139 | −2.057 | −0.817 | −0.536 | −1.265 | −0.885 | −1.364 | −2.374 | −2.141 | −0.457 | −1.503 | −1.28 |
| PPP1R16 [symbol] | 0.307 | 0.203 | −0.375 | −0.348 | −0.027 | 0.405 | −0.513 | −0.157 | −0.092 | −0.274 | 0.273 | −0.876 | −0.551 | −0.164 | −0.245 |
| PPP1R3C | −0.785 | −0.225 | −0.871 | 1.483 | 0.73 | 0.162 | 0.371 | −0.345 | −0.798 | −0.492 | 0.207 | 1.047 | −0.2 | 0.265 | 0.121 |
| PPP2R2D | 0.8595 | 0.3675 | −0.7055 | 4.3675 | −0.1015 | −1.9065 | −0.5875 | −0.1235 | 2.0935 | 3.2705 | 4.4325 | 2.4165 | 0.8075 | −0.5015 | 2.9115 |
| PPP2R5A | 0.421 | 0.872 | 0.443 | 0.684 | 1.055 | 0.687 | 0.874 | 0.263 | 0.637 | 0.917 | 0.991 | 0.958 | 0.528 | −0.307 | 0.486 |
| PRAME | 1.1175 | −0.1695 | 0.7115 | 0.4965 | 0.5915 | 1.1385 | 1.0715 | 1.0875 | 1.1615 | −0.2235 | 1.0125 | 1.3025 | 0.9875 | 0.9125 | 0.8485 |
| PRC1 | 0.091 | −0.487 | 0.513 | 0.862 | 0.1 | −0.181 | −0.067 | −0.207 | 0.317 | 0.237 | −0.383 | −0.18 | −0.19 | −0.285 | −0.186 |
| PRIM2A | −1.4895 | −1.8485 | 0.5245 | 0.8025 | −0.8105 | 0.1475 | −0.1975 | 0.1875 | 0.2425 | −0.0975 | 1.0005 | 0.2735 | 0.8805 | 0.2295 | 1.4545 |
| PRKACB | 0.724 | 0.797 | 2.148 | 2.187 | 0.354 | 0.225 | 0.189 | 0.165 | 1.009 | 0.823 | 0.756 | 0.102 | −0.195 | 0.409 | 0.824 |
| PRKCB1 | 0.145 | −0.22 | −0.431 | −0.256 | 0.972 | 0.202 | 1.177 | 0.982 | −0.09 | 0.898 | 1.458 | −0.116 | −0.263 | −0.039 | 0.869 |
| PRKCI | −0.341 | −0.294 | 0.979 | −0.68 | 0.217 | −0.068 | 1.185 | 1.1 | −0.677 | 0.76 | −0.037 | −0.211 | −0.638 | −0.527 | 0.757 |
| PRKRIR | 0.5125 | 1.8385 | 1.8705 | 1.8825 | 1.9155 | 1.2375 | 1.0385 | 0.0675 | 1.1695 | 1.2235 | 1.8985 | 1.1015 | 0.3925 | 0.7605 | 0.8675 |
| PRKWNK1 | 2.1525 | 1.0185 | 0.0885 | 1.1015 | 0.4065 | 0.9275 | 1.4445 | 0.6155 | 1.0505 | 0.8115 | −0.5825 | 2.5145 | 0.4725 | 0.0085 | −0.1655 |
| PRKX | −0.188 | 0.054 | 0.704 | 0.699 | −0.384 | −0.156 | −0.056 | 0.327 | 1.185 | 0.185 | 0.522 | −0.097 | 2.08 | 0.454 | 0.521 |
| PRNP | 0.1655 | −0.2775 | 0.1505 | 0.1655 | 1.1925 | 1.0035 | 0.1585 | 0.6665 | 0.1795 | 0.5595 | 0.5425 | 1.8425 | 1.8415 | 0.8855 | 1.2605 |
| PROL5 | 0.9285 | 1.0315 | 0.8525 | −0.2255 | 0.8735 | −0.0115 | 0.6855 | 0.5705 | −0.8595 | 0.8775 | 0.6185 | −0.1125 | −0.0945 | 1.0605 | 0.0255 |
| PRPF18 | 1.8495 | −1.3925 | 2.8385 | −0.8485 | 0.2415 | −0.4245 | 0.0095 | 1.9955 | 0.7725 | 0.2925 | −0.3985 | 0.0335 | 0.9885 | 0.7635 | 0.7105 |
| PRPSAP1 | 1.5805 | 1.2095 | −0.4845 | 0.7665 | −3.2895 | 0.2905 | 1.3335 | 1.2795 | 1.5575 | 0.5785 | 2.0345 | 2.1835 | 1.7295 | 1.4965 | 1.5845 |
| PRSS12 | −1.5075 | −1.3285 | −0.0315 | 0.0485 | 0.3765 | 0.8305 | 0.8525 | −0.1685 | 0.5975 | 0.1805 | 1.8335 | −1.4225 | −1.0785 | −0.0935 | −0.9345 |
| PRSS16 | −0.0475 | −0.2135 | 0.4075 | 0.7215 | 0.1945 | 0.7375 | 0.5315 | −0.0735 | −0.3845 | −0.4295 | −0.3425 | 0.1775 | 0.6445 | 0.2235 | 0.5495 |
| PSD3 | −0.1715 | 0.0885 | 0.8105 | 0.3805 | 0.7855 | 0.7815 | −0.5735 | 0.5185 | 0.2825 | −0.3475 | −0.7465 | 0.6225 | 0.4625 | 0.5065 | 1.4475 |
| PSMA3 | −0.276 | −0.488 | −0.946 | −1.275 | −0.052 | −0.383 | −0.295 | −0.643 | −0.252 | −0.227 | −0.446 | −0.941 | −0.654 | −0.031 | −0.646 |
| PSMA7 | 0.15 | −0.517 | −0.275 | −0.047 | −0.261 | 0.408 | 0.228 | −0.217 | −0.089 | −0.076 | −0.811 | −0.234 | −0.093 | −0.166 | −0.212 |
| PSMC5 | 0.7645 | −0.3495 | 0.2165 | −0.0005 | −0.9365 | 0.5425 | 0.8725 | 0.6645 | 0.2535 | 0.0205 | 0.6035 | 1.1435 | −0.2445 | 1.1665 | 1.4765 |
| PSMC6 | 0.822 | −0.421 | −0.454 | −0.002 | 0.729 | 0.013 | 1.063 | 0.87 | 0.176 | 0.293 | 0.158 | 0.752 | 0.488 | 1.521 | 0.741 |
| PSMD12 | −0.7105 | −0.3265 | 0.2975 | −0.3335 | −0.5405 | 0.0585 | −0.6545 | −0.8565 | 0.0895 | −0.4445 | −0.2545 | −0.2985 | −0.5625 | −0.3195 | −0.6165 |
| PSMD12 | 1.1145 | 0.1385 | −0.4505 | −0.6125 | −0.6005 | 1.4905 | −1.1245 | −1.2215 | 0.0975 | 0.1255 | 0.1685 | 1.1505 | 1.8775 | 1.5055 | 0.2775 |
| PSMD3 | −0.8325 | 0.7005 | −1.2515 | 0.4785 | 0.5265 | 0.4255 | 1.1095 | −0.5285 | 0.3515 | 0.8915 | 0.1195 | 0.5485 | 0.5365 | 0.2635 | −0.8845 |
| PSPH | 0.384 | −0.616 | −0.821 | 0.628 | 0.083 | −0.288 | −0.709 | −0.581 | 0.381 | −0.594 | −0.73 | −0.538 | −0.324 | −0.232 | 0.098 |
| PTD012 | −0.58 | −0.385 | −0.897 | −0.039 | −0.23 | −0.164 | −0.137 | −0.183 | 0.405 | 0.144 | −0.155 | −0.532 | 0.097 | −0.321 | 0.053 |
| PTGER3 | 0.543 | 0.385 | 0.26 | 0.393 | 1.502 | −0.252 | −0.761 | −0.781 | −0.091 | −0.965 | −1.158 | −0.745 | −0.404 | 0.455 | −0.647 |
| PTHLH | −0.0115 | −0.2495 | 0.1785 | 0.4545 | 0.5075 | 0.1055 | 0.2185 | 0.0945 | 0.2005 | 0.0965 | 1.1785 | −0.1235 | −0.3855 | −0.1565 | −0.3935 |
| PTK6 | −0.232 | −1.186 | 0.322 | 0.065 | −0.175 | 0.029 | −0.291 | 0.118 | 0.432 | 0.115 | 0.783 | −0.114 | 0.277 | −0.412 | 0.399 |
| PTPN4 | 1.748 | 0.905 | 0.927 | 0.703 | 1.032 | 0.998 | −0.093 | 0.88 | −0.193 | 0.293 | 1.185 | −0.396 | −0.641 | 0.032 | 0.327 |
| PTPN7 | −1.057 | −1.253 | 0.388 | −0.128 | −0.53 | −0.053 | 0.344 | −0.3 | 0.484 | −0.199 | −1.255 | −0.418 | 0.81 | −0.618 | 0.093 |
| PTPRF | −0.727 | −0.948 | −0.759 | −0.694 | −0.633 | 0.27 | −0.057 | −0.111 | −0.489 | −0.478 | −0.212 | −0.135 | −0.281 | −0.599 | −0.308 |
| PTPRN2 | 0.4045 | 0.1885 | 0.1515 | 0.7015 | 0.3895 | 0.8805 | 1.8685 | 1.2305 | 0.9535 | 1.3325 | −0.5485 | 1.0085 | 1.4945 | 0.9935 | 0.0795 |
| PTPRT | 8.3045 | −0.0485 | 0.8425 | 0.5876 | 1.4775 | 1.1315 | 0.9335 | 0.6185 | −0.0265 | 0.3225 | 0.3775 | −0.1065 | 0.4825 | 0.0575 | 0.5155 |
| PTS | 1.276 | 0.145 | 0.121 | −0.557 | 1.025 | 0.33 | 0.028 | −0.493 | 1.012 | 0.222 | 0.3 | 1.035 | 1.323 | 0.575 | 0.668 |
| PTTG1 | 0.4805 | −0.5155 | 0.5195 | −1.6015 | −0.2395 | 0.1945 | −0.4125 | 0.0825 | 0.3995 | −0.0245 | 0.7175 | 0.0355 | −0.2005 | 0.8255 | 1.1795 |
| PURB | −0.7915 | −1.0375 | 1.2875 | −0.0315 | −0.1855 | −1.1035 | −2.4395 | −1.0275 | −0.8475 | −1.6685 | −0.7065 | −0.5395 | 0.5205 | −0.6695 | 0.5875 |
| PXMP2 | 0.6035 | 0.4595 | 0.9815 | −0.0405 | 0.5685 | 0.3905 | 0.5505 | 0.6795 | −0.8495 | −0.7925 | −0.0965 | 0.1065 | −0.2725 | 1.2155 | 1.4275 |
| PYCARD | −0.736 | −0.831 | −1.129 | −2.112 | −0.688 | −0.006 | 0.119 | −0.429 | −0.601 | −1.138 | −1.064 | −0.342 | −0.061 | 0.723 | −0.233 |
| PYCR1 | −1.538 | −1.137 | −0.81 | −1.142 | 0.844 | 0.563 | −1.241 | −1.309 | −0.412 | −0.553 | −1.566 | 1.129 | 0.891 | 1.698 | −0.972 |
| QDPR | −0.4215 | −1.2295 | −1.8015 | 0.5845 | −1.1035 | 0.3525 | −0.8595 | 0.2495 | −0.2155 | −1.1095 | 0.5835 | −0.0385 | −0.0255 | 1.0085 | −0.0685 |
| QPRT | 0.171 | −1.875 | 0.926 | −0.357 | 0.202 | −0.884 | −1.032 | −0.624 | −0.808 | −0.883 | −1.7 | 0.559 | −0.075 | −0.27 | −1.249 |
| QSCN9 | −1.1655 | −1.7285 | −1.1765 | −1.1535 | 0.9525 | 0.1385 | 0.2805 | −0.6685 | −0.8905 | −0.3745 | −0.5115 | 0.4095 | −0.4125 | −0.2315 | −0.2995 |
| RAB11FIP | −0.538 | −0.417 | −0.377 | −0.225 | 0.627 | 0.534 | −0.442 | 0.29 | 0.116 | −0.463 | −0.382 | −0.988 | 0.154 | 0.092 | −0.6 |
| RAB11FIP | 0.916 | 0.826 | 0.341 | 1.11 | 1.253 | 1.073 | −0.016 | 0.202 | 0.402 | −0.773 | 0.216 | 1.223 | 1.206 | 0.756 | 0.207 |
| RAB13 | −0.8 | −0.592 | −0.383 | −0.148 | 0.203 | −0.304 | −0.45 | −0.418 | 0.105 | −0.093 | −0.596 | −0.599 | −0.115 | −0.532 | −0.282 |
| RAB27A | −1.113 | −0.931 | −0.619 | −0.67 | −1.392 | −0.552 | −0.145 | 0.084 | 0.661 | −0.377 | −0.494 | −1.726 | 0.303 | 0.379 | 0.432 |
| RAB30 | 0.5935 | −0.5125 | 0.4455 | 0.3485 | 0.6255 | 0.6625 | −0.2255 | 0.1185 | −0.1795 | −0.1735 | 0.2185 | 0.8715 | 0.6405 | 1.1495 | 0.9095 |
| RAB34 | −0.679 | −1.191 | 0.172 | −0.329 | −0.513 | −1.62 | −0.457 | −0.04 | −0.68 | −0.468 | −0.328 | 0.043 | −0.036 | −0.385 | −0.048 |
| RAB35 | 0.048 | 0.121 | 0.181 | −0.18 | −0.011 | 0.164 | −0.156 | 0.003 | −0.025 | −0.162 | −0.323 | −0.096 | −0.05 | 0.228 | 0.222 |
| RAB39B | −0.0385 | 0.1355 | 0.1535 | −1.0265 | −2.2616 | −1.1805 | −0.0815 | 1.4635 | −0.7035 | −0.8005 | 0.5465 | 0.7235 | 0.3015 | −0.1745 | −0.4565 |
| RAB3A | 0.104 | −0.319 | 0.216 | −1.298 | −0.358 | −1.232 | 0.063 | 0.797 | −1.273 | −1.705 | −1.016 | −1.088 | −0.623 | −0.03 | −1.285 |
| RAB3IP | −0.451 | 0.498 | 0.292 | 0.159 | 0.996 | 0.001 | −0.064 | 0.785 | −0.153 | 0.805 | 0.285 | 0.741 | 0.345 | 1.132 | 0.492 |
| RABEP1 | 0.461 | 0.042 | 0.815 | 0.765 | 0.522 | 0.605 | 0.862 | 1.102 | 0.147 | −0.485 | 0.618 | 1.078 | 0.376 | 0.506 | 0.284 |
| RABGAP1 | −0.7725 | −0.7975 | −1.1905 | −0.4295 | −0.4515 | −0.2465 | −0.1405 | −0.0245 | −0.1845 | 0.2905 | −0.0625 | −0.1885 | −0.1835 | −0.4745 | 0.2215 |
| RACGAP1 | 0.0695 | 0.4855 | 0.6835 | 0.5355 | 0.8555 | 0.0485 | 0.9325 | 0.7205 | −0.0465 | −0.0585 | 0.1975 | 0.7875 | 0.5005 | −0.4365 | 1.0775 |
| RAD17 | 0.435 | 0.14 | 2.057 | 0.657 | 0.521 | 0.694 | 0.358 | 0.918 | 0.817 | 0.421 | 0.991 | 1.201 | 0.593 | 0.801 | 0.961 |
| RAD51 | −0.293 | −0.285 | 0.702 | −0.062 | 0.09 | −0.415 | −0.012 | −0.049 | 0.602 | 0.277 | −0.548 | −0.148 | 0.712 | −0.187 | 0.307 |
| RAD51AP | 0.7245 | −0.1235 | 0.9485 | 0.6255 | 1.7945 | 0.5655 | 0.0275 | 0.6655 | 0.7415 | 0.7895 | 1.5045 | 0.2955 | 1.3675 | 0.1415 | 0.9055 |
| RAD52 | −0.810 | 0.401 | 1.09 | 1.095 | 0.718 | 0.564 | 0.792 | 0.601 | 0.374 | 0.522 | 1.494 | 1.204 | −0.516 | 0.374 | 0.874 |
| RAD54L | −0.5315 | −0.7365 | −0.4835 | 0.0835 | 0.7425 | 1.3955 | 0.4435 | 0.5205 | −0.0085 | 0.0965 | 1.5035 | 0.6305 | −0.2385 | 0.2965 | 0.7855 |
| RAMP | 3.8075 | 1.9375 | 4.9495 | 4.0105 | 1.9855 | 2.3055 | −0.1645 | 0.8945 | 2.9525 | −0.7945 | 3.7555 | 2.5405 | 2.3775 | 3.5416 | 1.3685 |
| RANBP1 | 0.3245 | −0.8125 | 0.4645 | 0.1875 | −0.0845 | 0.5225 | 1.1145 | −0.4285 | 0.0735 | −0.4195 | −0.0255 | 0.2175 | 0.4425 | 0.3365 | 0.0045 |
| RARRES1 | 1.787 | 0.66 | 0.852 | 0.238 | −0.34 | −0.17 | −0.637 | 0.067 | −0.059 | −0.097 | 0.313 | −0.434 | −1.052 | −0.333 | 0.756 |
| RBBP7 | 0.8285 | −0.3485 | 0.5385 | −0.0595 | 1.2555 | 0.7795 | 1.0225 | 0.9655 | 0.2945 | −0.4325 | 0.7416 | 0.5885 | 0.7955 | 0.9535 | 0.1985 |
| RBBP8 | −0.4755 | 0.5465 | 0.5125 | 0.3515 | 0.5555 | 0.3595 | 0.8405 | 0.2265 | −0.5565 | 0.0225 | 0.6205 | 0.0485 | 0.2815 | −0.8155 | −0.3935 |
| RBM8A | −1.118 | −0.837 | −0.517 | −0.701 | −1.081 | −0.66 | −0.673 | 0.048 | −0.379 | −0.19 | −0.298 | −0.905 | −0.155 | −0.755 | −0.559 |
| RBX1 | −0.114 | −0.356 | 0.622 | −0.183 | −0.252 | −0.044 | −0.824 | 0.272 | −0.274 | 0.661 | 0.091 | −0.243 | −0.698 | 0.392 | −0.708 |
| RDHE2 | −0.467 | −0.615 | −0.801 | 0.024 | −0.151 | 0.19 | −0.134 | −0.408 | −0.212 | 0.015 | −0.157 | 0.654 | 0.346 | −0.896 | −1.04 |
| REA | 0.073 | −0.2 | −0.402 | −0.079 | −0.399 | −0.019 | 0.04 | 0.269 | 0.159 | −0.053 | −0.136 | 0.363 | 0.362 | 0.135 | 0.244 |
| REC14 | −0.0535 | 0.7655 | 2.5245 | 0.9185 | 0.3655 | 0.2295 | −0.3165 | 0.8255 | 1.1455 | −0.1435 | 0.8505 | 0.2615 | 0.6175 | −0.0175 | 1.5695 |
| REG1A | −0.298 | 0.189 | −0.576 | −0.093 | −1.591 | −0.375 | 0.838 | 0.254 | −0.22 | 0.168 | −0.731 | 0.738 | 0.312 | 0.28 | 0.719 |
| RELB | −0.027 | −1.197 | −0.044 | 0.256 | 1.44 | −0.008 | −0.203 | −0.584 | 0.078 | −0.203 | −1.172 | 0.228 | −0.432 | 0.412 | −0.68 |
| REPIN1 | −1.136 | −0.62 | 0.195 | −0.883 | −1.482 | −0.77 | −0.949 | −0.416 | 0.29 | 0.25 | 0.355 | −1.198 | −1.109 | −0.702 | −1.332 |
| REPS2 | 0.0385 | 0.1195 | 0.3295 | 0.7315 | 0.5585 | 1.0895 | 0.1255 | 1.1235 | 1.3855 | 0.9315 | 1.3355 | 1.0695 | 1.1945 | 0.5805 | 0.3455 |
| RERG | 0.2305 | 0.7015 | 1.5405 | 0.0575 | 0.9155 | −0.0875 | 0.2415 | 0.7315 | −0.1385 | 0.5355 | 0.4655 | 1.9475 | 1.7825 | −0.1805 | −0.0325 |
| RFC4 | 1.2615 | 0.0445 | 2.0845 | 1.0585 | 0.0065 | 0.6795 | −0.5745 | 0.7545 | 0.0205 | −0.1505 | 1.2185 | 1.2615 | 0.2225 | 0.8015 | 2.2455 |
| RGS10 | 0.4335 | −0.7185 | 0.3226 | 1.2815 | 0.5885 | 1.6195 | −0.5865 | −0.7305 | 0.0105 | −0.1945 | 0.3405 | −0.0315 | −0.7295 | 0.0425 | −0.0605 |
| RH8DL2 | 0.42 | −0.206 | −0.126 | −0.54 | −0.414 | −0.163 | −0.258 | −0.029 | 0.232 | 0.006 | −0.079 | −0.009 | 0.227 | 0.356 | 0.124 |
| RIBC2 | 0.655 | −0.817 | −0.138 | 0.052 | 0.771 | 0.087 | 0.915 | 0.873 | 0.295 | −0.07 | −0.519 | 2.168 | 0.957 | 1.339 | 0.667 |
| RIC-8 | 0.658 | −1.275 | −0.705 | −0.267 | −1.029 | −0.358 | 0.173 | −0.486 | 0.422 | 0.361 | 0.045 | −0.779 | −0.014 | −0.533 | 0.052 |
| RIT1 | 0.397 | −0.885 | −1.04 | −0.707 | −0.28 | −0.151 | −0.74 | −0.692 | −0.888 | 0.516 | 0.045 | −0.573 | −0.348 | −0.063 | −0.1 |
| RLN2 | −0.818 | −0.059 | −0.111 | −0.387 | −0.329 | −0.041 | −0.508 | −0.417 | 0.25 | −0.432 | −1.225 | −0.499 | −0.89 | 0.275 | 0.527 |
| RNF10 | −0.1925 | −0.6585 | −0.2745 | −0.1525 | 0.1135 | −0.1125 | 0.4835 | −0.2155 | 0.7375 | −0.0395 | 0.1575 | −0.2585 | 0.9155 | 0.1885 | 0.3485 |
| RNF135 | 3.4705 | 2.9885 | 3.7805 | 3.8045 | 3.8865 | 5.8655 | 3.6846 | 3.0335 | 2.2635 | 3.2305 | 4.4735 | 3.9935 | 2.8025 | 3.4015 | −0.0075 |
| RNF7 | 0.232 | −0.297 | −0.309 | 0.034 | −0.407 | 0.208 | −0.377 | −0.643 | −0.283 | −1.024 | 0.257 | 0.278 | −0.304 | −0.208 | −0.342 |
| ROPN1 | 0.0945 | −1.8495 | −2.3375 | 3.5785 | −0.7255 | −0.4455 | −0.0575 | −1.0725 | 1.0605 | −1.3935 | 0.9255 | −0.1605 | −0.8975 | 2.8095 | −1.1425 |
| RPA3 | −0.1895 | 0.2005 | −0.4965 | 0.0915 | −0.0185 | 0.1805 | −0.2995 | −0.0845 | 0.2575 | 0.5495 | 0.3915 | −0.8175 | −0.1795 | 0.2415 | 0.4885 |
| RPESP | −0.882 | 0.722 | −0.372 | −0.013 | −0.352 | 0.846 | −0.181 | 0.923 | 0.284 | 1.116 | −0.119 | −0.382 | −0.539 | −0.451 | 0.17 |
| RPL11 | −0.061 | 0.593 | −0.352 | −0.087 | −0.513 | 0.283 | −0.148 | 0.025 | 0.456 | 0.372 | 0.462 | −1.503 | −1.207 | −0.9 | −0.35 |
| RPL13 | −1.041 | −0.598 | −0.38 | −0.619 | −1.573 | 0.059 | −0.262 | −0.167 | −0.221 | −0.242 | −0.252 | 0.815 | −0.99 | −0.315 | −0.742 |
| RPL13A | 0.8815 | 1.6485 | 1.2545 | 0.7965 | 0.3775 | 0.6145 | 0.0395 | −0.3005 | 0.3765 | 0.8025 | 0.5325 | −0.8965 | −0.2615 | −0.3835 | −0.0935 |
| RPL15 | −0.721 | −0.653 | −0.096 | 0.176 | −1.017 | −0.094 | −0.097 | −0.285 | 0.142 | 0.381 | −0.002 | 0.144 | 0.093 | 0.116 | 0.126 |
| RPL18A | −1.452 | 0.811 | −0.007 | 0.059 | −0.575 | 0.04 | 0.168 | 0.187 | −0.158 | 0.512 | 0.824 | 0.119 | −0.022 | 0.184 | 0.152 |
| RPL19 | −1.178 | −0.489 | −0.729 | −0.556 | −0.841 | −0.411 | −0.353 | −0.08 | 0.593 | 0.174 | −0.456 | −0.695 | −0.016 | −0.167 | 0.883 |
| RPL21 | −1.0875 | −0.5585 | −0.8855 | −0.3585 | −0.8295 | −0.3125 | −0.4565 | −0.2015 | −0.0775 | 0.3025 | −0.5255 | −0.7765 | 0.0015 | −0.1905 | 0.3865 |
| RPL23A | −0.195 | 0.205 | −0.179 | 0.414 | −0.253 | 0.557 | 0.032 | −0.008 | 1.016 | 0.827 | −0.099 | −0.743 | −0.28 | 0.538 | 0.075 |
| RPL23A | −0.3485 | −0.2755 | −0.2305 | −0.3955 | 0.7425 | −0.4175 | −0.0705 | −0.6845 | −0.5265 | 0.1125 | −0.8015 | −0.3725 | −0.9685 | −0.1895 | −0.7555 |
| RPL26L1 | −0.1545 | 0.2555 | −0.0615 | 0.0655 | 0.8885 | 0.3365 | −0.3795 | −0.3195 | 0.2005 | 1.1595 | 0.3535 | −0.9365 | −0.7595 | −0.6405 | −0.1485 |
| RPL29 | 0.1115 | −0.5605 | −0.1815 | −0.0785 | 0.0695 | 0.4965 | −0.1055 | −0.3495 | 0.1375 | 0.1875 | −0.5515 | −0.9455 | −0.3975 | −0.5445 | −0.1845 |
| RPL35 | 0.0275 | 0.1525 | 0.0935 | 0.1855 | 0.9105 | 0.5475 | 0.1055 | 0.7655 | −0.1245 | 0.2295 | 0.3645 | −0.7885 | −0.0795 | −0.5415 | 0.1185 |
| RPL36AL | −1.0055 | −0.3665 | −0.4195 | −0.6045 | 0.0385 | −2.4695 | −0.3525 | −0.2935 | −0.3405 | 0.0895 | −1.6265 | −0.1385 | 0.0035 | −0.1865 | −0.0695 |
| RPS16 | 0.7465 | −0.0025 | −0.4845 | −0.1285 | 0.3105 | −0.0115 | −0.0685 | −0.1995 | −0.3185 | 0.1475 | 0.1355 | 0.2605 | −0.1415 | 0.8005 | 0.7945 |
| RPS4Y1 | 0.2455 | −1.4005 | 0.2885 | 1.1125 | 2.7525 | 0.8485 | 1.0635 | 0.5575 | 0.1615 | −0.3425 | 0.5385 | 3.5605 | 1.7025 | 1.8735 | −0.6355 |
| RRAGA | 0.822 | 0.093 | 0.94 | −0.102 | 0.457 | 0.845 | 0.156 | 0.158 | 0.717 | 0.605 | 1.086 | 0.403 | −0.498 | 0.738 | −0.178 |
| RRAGD | −0.291 | −1.517 | 0.505 | 0.163 | 0.778 | 0.821 | 0.662 | 0.154 | −0.013 | 0.259 | 0.258 | −0.122 | 0.685 | 0.738 | 0.751 |
| RRM1 | −0.563 | 0.203 | −0.287 | −0.114 | 0.288 | 0 | 0.234 | 0.409 | −0.131 | 0.051 | 0.106 | 0.352 | 0.286 | 0.101 | −0.024 |
| RRM2 | 1.802 | −1.282 | 0.677 | 1.421 | −2.672 | 0.364 | 0.351 | 1.076 | 2.707 | 0.849 | 0.673 | 3.095 | 1.808 | 0.786 | −0.44 |
| RSAFD1 | 0.7515 | 0.2345 | 0.5905 | 0.7135 | 1.9625 | 1.4705 | 0.9835 | 1.0675 | 0.6155 | 1.2135 | 0.2915 | 1.4725 | 1.3455 | 0.9325 | 0.4515 |
| RSNL2 | −0.1975 | −0.4185 | 0.5475 | 1.5275 | 0.6355 | 0.8585 | 1.8175 | 0.2695 | 1.0815 | 0.7035 | 1.1485 | 1.2255 | 1.6505 | 1.0385 | −0.5935 |
| RSU1 | 1.809 | −0.689 | 0.651 | 0.203 | 0.391 | −0.247 | 0.333 | 0.474 | 0.477 | −0.26 | −0.393 | 1.023 | 0.856 | 0.707 | −0.01 |
| RTN4IP1 | −0.004 | −0.117 | 1.984 | −1.517 | −2.65 | −0.625 | −3.768 | −2.718 | −1.731 | −1.605 | −1.782 | −1.168 | −0.059 | 0.937 | 0.225 |
| S100A11 | 2.191 | 0.129 | 1.172 | −0.888 | −2.192 | −0.979 | −1.594 | −0.826 | 0.679 | −0.501 | −1.013 | −1.661 | 0.273 | 0.829 | −2.031 |
| S100A14 | 2.5245 | −0.4685 | 1.4275 | 3.2795 | 0.8795 | 0.2435 | 0.7275 | 3.0725 | −0.4085 | −1.5025 | −0.3615 | 3.2125 | 2.9975 | 4.3835 | −0.0845 |
| S100A16 | 0.0195 | −0.5955 | −0.4885 | 0.2055 | −0.2955 | 1.5535 | 0.1805 | −0.1245 | 0.1975 | −0.1995 | 0.4455 | −0.4965 | −0.0075 | −0.6405 | 0.0885 |
| S100A9 | −1.285 | −1.489 | −0.499 | −1.96 | −1.504 | 0.016 | −0.400 | −0.417 | −0.182 | −0.399 | −0.175 | −0.509 | −0.783 | −1.282 | −0.321 |
| SAE1 | 0.5045 | −0.4285 | −0.0015 | 0.4955 | 1.0035 | 0.9795 | 0.4455 | −0.3015 | 0.4435 | 0.1035 | 0.0145 | −0.8095 | 0.2055 | 0.8365 | 0.0135 |
| SALL2 | −0.223 | −0.033 | −0.447 | 0.409 | 0.08 | −0.358 | −0.47 | −0.684 | −0.359 | −0.451 | −1.583 | 0.137 | −0.039 | 0.496 | −0.153 |
| SARA1 | −0.7065 | −0.3455 | −0.8505 | −0.7705 | −0.6305 | −0.1755 | −0.2825 | −0.1385 | −0.1855 | 0.0195 | −0.7355 | −0.3605 | −0.4775 | −0.7615 | −0.2715 |
| SARA2 | 0.908 | −0.537 | 0.117 | −0.191 | 0.808 | 0.883 | 0.809 | −0.185 | −0.324 | −0.226 | 0.994 | 0.147 | −0.201 | −0.041 | −0.358 |
| SAS | 0.285 | −1.488 | −0.834 | 0.322 | 0.661 | 0.424 | −0.859 | −0.44 | 0.094 | −0.872 | 0.029 | −0.735 | −0.419 | 1.301 | −0.658 |
| SCIN | 0.6375 | −0.7585 | 0.1545 | 0.1665 | 0.8835 | 0.0715 | −0.1355 | 0.1805 | 0.2275 | −0.2165 | 0.4925 | 0.1425 | −0.1535 | 0.5865 | 1.6095 |
| SCML1 | 0.896 | 0.022 | 0.943 | 0.358 | 1.317 | 0.543 | 0.577 | −0.509 | 0.135 | 0.119 | −0.199 | 0.701 | 0.226 | 0.088 | 0.454 |
| SCPEP1 | 0.1445 | −0.8355 | −0.4855 | −0.0385 | 0.8325 | −0.4365 | 0.7415 | 0.0155 | 0.4315 | 0.7245 | −0.2565 | 1.0145 | 1.8535 | −0.0805 | −0.4475 |
| SCRIB | 0.052 | −0.97 | −0.761 | −0.126 | −0.134 | 0.055 | 0.407 | 0.34 | −2.067 | −0.301 | −0.085 | 0.254 | −0.561 | 0.519 | 0.312 |
| SDC1 | −0.179 | 0.421 | −0.763 | −0.288 | 0.061 | 0.854 | 0.736 | 0.734 | 0.138 | −0.01 | 0.627 | 0.89 | 0.06 | 0.693 | 0.493 |
| SDC3 | 0.6695 | 0.6805 | 0.8935 | 0.2615 | −0.1395 | 0.3145 | 0.3515 | 0.2915 | 0.3045 | 0.2165 | 0.1095 | 0.3785 | 0.2705 | 0.2765 | 0.0125 |
| SDCCAG0 | 0.3095 | −0.8315 | −0.8335 | −0.0095 | 0.3475 | 0.2595 | 0.5055 | 0.2525 | −0.0385 | −0.1285 | 0.2005 | 0.2735 | 0.9695 | 0.4755 | 0.1545 |
| SEC61G | 0.037 | −0.691 | −0.98 | −0.452 | −0.11 | 0.257 | 0.493 | −0.176 | −0.845 | −0.298 | −0.294 | −0.428 | −0.364 | −0.327 | 0.199 |
| SECP43 | 1.0805 | −1.0715 | −1.7825 | −1.7475 | −0.8455 | −1.6535 | −0.4085 | −1.0905 | 0.3045 | −0.7915 | −1.2785 | −1.8875 | −0.6755 | 0.7615 | −1.5785 |
| SEDLP | −1.107 | −1.089 | −2.612 | −0.575 | −3.165 | −0.774 | −1.159 | −1.734 | 0.172 | −0.528 | −2.867 | −2.37 | −2.698 | −2.27 | −2.337 |
| SEMA3B | 0.5595 | −0.8285 | −0.8075 | −0.1855 | −0.7355 | 1.0275 | −0.2935 | −0.2575 | −0.1095 | −0.1835 | −0.7775 | −0.6915 | −0.4915 | −1.0565 | −0.9005 |
| SEMA3C | 0.0285 | 0.3935 | 1.0535 | 0.2215 | −0.8076 | −0.3475 | 0.2775 | −0.3295 | 0.4525 | 0.3815 | −0.2445 | −0.0905 | 0.1205 | −0.3055 | 0.6575 |
| SEPW1 | −1.953 | 0.959 | −1.085 | −1.234 | −0.79 | −1.392 | −1.769 | −1.019 | −0.365 | −1.022 | −2.239 | 0.378 | −0.08 | 2.405 | −1.541 |
| SFRP1 | 2.8585 | 1.0065 | 2.8195 | 3.0375 | −0.8485 | 0.1705 | 0.2045 | 2.2235 | 0.5175 | 0.6225 | 0.9755 | 1.1535 | 2.2875 | 2.3605 | 0.3805 |
| SERPINA [symbol] | 1.624 | 1.511 | 1.007 | 2.734 | 2.57 | 2.588 | 3.231 | 2.729 | 2.184 | 1.71 | 3.827 | 1.686 | 1.743 | 0.733 | 1.833 |
| SERPINB [symbol] | −1.258 | −0.611 | −0.039 | −1.376 | −0.309 | −0.317 | −0.554 | −0.838 | 0.737 | 0.353 | −0.119 | −0.402 | −0.155 | 0.278 | −0.517 |
| SFRP1 | −0.773 | −0.007 | −1.259 | −0.474 | 0.756 | −0.566 | −0.073 | 0.308 | 1.049 | 0.232 | −0.875 | −0.484 | 0.45 | −0.747 | −0.452 |
| SFRS6 | 0.2505 | 1.2825 | 0.2055 | 0.8285 | 0.0265 | 0.5875 | 1.3405 | 1.3435 | 0.9405 | 1.0455 | 0.5045 | 1.8585 | 0.9745 | 0.7416 | −0.9605 |
| SGCD | −0.5715 | −0.2835 | −1.5385 | −1.3095 | −0.4575 | −0.6575 | −0.1025 | −0.5995 | −0.6765 | −0.2085 | −1.1355 | −0.2175 | −0.3875 | 0.0345 | 0.0605 |
| SGCE | 0.584 | 0.659 | 0.682 | 0.545 | 0.722 | 0.744 | 1.204 | 0.423 | 0.188 | 0.493 | 0.171 | 0.223 | 0.529 | 0.355 | 0.668 |
| SH3BGRL | 0.323 | −1.719 | −1.479 | −1.834 | −1.552 | −0.589 | −0.027 | −0.334 | −1.019 | −0.431 | −1.067 | 0.985 | 0.022 | −0.87 | −1.716 |
| SHFM1 | −0.1645 | 0.1355 | −0.0085 | 0.0505 | 0.4535 | −0.1016 | −0.4935 | −0.2625 | 0.6865 | 0.6395 | 0.2765 | −0.6795 | 0.8545 | −0.2555 | 0.0115 |
| SIAT7B | −0.79 | 0.438 | 0.687 | 1.069 | −0.624 | 0.013 | −0.892 | −0.181 | 1.084 | 0.117 | 0.958 | −0.359 | 0.412 | −0.381 | 0.886 |
| SIGLEC12 | −0.06 | 0.842 | 0.409 | −0.289 | 0.267 | 0.727 | −0.579 | −0.715 | 0.399 | 0.075 | 0.163 | −1.537 | −0.308 | −0.778 | −0.683 |
| SIT | −0.9575 | −0.4245 | −0.8735 | −0.2865 | −0.8695 | −0.0595 | −0.1255 | −0.1635 | −0.4985 | −0.3415 | −1.0405 | 0.2525 | −0.2235 | −0.5315 | −0.4395 |
| SITPEC | 0.0545 | 0.4555 | −0.1495 | 0.3435 | 0.6395 | −0.0676 | 0.6765 | −0.1905 | 0.4375 | 0.3095 | 0.3555 | −0.4855 | 0.0595 | −0.1925 | 0.0435 |
| SKP1A | 0.4005 | 1.7185 | −0.5425 | 0.6865 | 1.4245 | 0.0665 | 0.9555 | 1.0795 | 0.5835 | 0.1475 | 1.0035 | 3.4405 | 1.5305 | 1.5895 | 2.6915 |
| SLC12A9 | −1.755 | −1.467 | −0.724 | −1.173 | −2.312 | −1.581 | −0.931 | −0.688 | −1 | −1.108 | −1.457 | −0.605 | −0.783 | −0.558 | −1.361 |
| SLC13A3 | −1.7705 | −1.0516 | −0.6525 | −0.1315 | −0.0965 | −0.9605 | −0.0065 | −0.5155 | −1.3025 | 0.0955 | −0.7095 | 0.2845 | −0.2405 | 0.5295 | −0.6775 |
| SLC16A6 | 1.238 | −0.178 | −1.785 | −1.12 | −1.546 | −1.09 | −0.004 | −0.471 | 0.004 | −0.934 | −1.166 | −0.23 | −0.80 | −1.259 | −2.773 |
| SLC19A2 | −1.4375 | 1.1305 | 0.3375 | 0.7005 | −0.3935 | −1.5305 | −1.4645 | −1.9265 | −1.1525 | −0.7095 | 0.5345 | −1.5045 | −0.7735 | 0.0395 | −1.2875 |
| SLC1A1 | 0.4755 | −0.8645 | −0.9685 | 0.4225 | −0.9175 | −0.4325 | 0.9625 | 0.3465 | 0.6115 | 0.0375 | −0.7065 | 2.4335 | 0.3625 | 1.4625 | −0.0335 |
| SLC1A2 | 0.1945 | 0.1645 | 0.2285 | 0.2985 | −0.1255 | 0.3345 | −0.9515 | −1.0445 | −0.8985 | −0.3865 | −0.4175 | −1.0585 | −1.0555 | 0.0816 | 0.1375 |
| SLC24A3 | −0.663 | −0.161 | −1.438 | −0.421 | −3.708 | −0.272 | 0.415 | −0.001 | −0.644 | −0.629 | 0.836 | 0.811 | −0.283 | 0.098 | 0.118 |
| SLC25A29 | 0.87 | −0.483 | −0.158 | 0.287 | 0.458 | 1.050 | 0.828 | −0.381 | 0.468 | −0.178 | 0.135 | 0.852 | −0.142 | 1.416 | 0.448 |
| SLC25A4 | 0.523 | −1.183 | −0.63 | −0.996 | −0.411 | −1.281 | −0.849 | −0.826 | −0.851 | −1.014 | −0.897 | −1.224 | −0.806 | −1.548 | −1.288 |
| SLC25A5 | 1.1085 | 0.9775 | 0.2205 | 1.0625 | 0.6565 | 1.4795 | 0.1305 | −0.6455 | 0.3945 | 0.4825 | 0.5915 | −1.8975 | −1.0055 | −0.4505 | 0.1415 |
| SLC27A2 | −0.427 | −0.532 | −0.2 | −0.506 | −0.323 | −0.494 | −0.138 | −0.004 | −0.394 | 0.056 | −0.525 | 0.262 | −0.184 | −0.14 | −0.513 |
| SLC27A5 | 1.677 | 1.269 | 0.724 | −0.143 | 1.208 | 1.847 | 1.415 | −0.476 | −0.84 | −0.267 | −0.172 | 1.003 | −0.843 | −0.01 | −0.477 |
| SLC30A8 | −0.7215 | −0.7315 | −1.4665 | −1.3085 | −1.9125 | −1.3195 | −1.7825 | −1.0305 | −1.6025 | −1.0755 | −2.2335 | −2.1605 | −1.3085 | 0.3985 | −0.3745 |
| SLC39A4 | −0.605 | −2.74 | −1.058 | 0.135 | −3.117 | 0.103 | −1.241 | −2 | −0.831 | −2.032 | −1.4 | −1.394 | −2.083 | −2.254 | −1.606 |
| SLC39A6 | 0.0595 | −0.7675 | 0.0645 | −0.7775 | 0.1525 | 0.4095 | −0.4055 | 0.3345 | 0.5105 | 1.1355 | 0.6645 | 0.6505 | −0.0155 | 0.6565 | 0.6435 |
| SLC40A1 | −0.4425 | 0.2455 | 1.0055 | 1.0905 | 0.5335 | −0.2965 | −0.8475 | −0.0885 | −0.0165 | −0.9505 | 1.1045 | 1.2175 | 0.5775 | 0.5075 | 1.4945 |
| SLC41A3 | −0.613 | −0.4 | −0.702 | −0.55 | 0.419 | −0.331 | 0.152 | −0.045 | −0.121 | −0.146 | −0.839 | 0.058 | 0.155 | −0.091 | 0.111 |
| SLC5A6 | 0.6385 | −1.8525 | −1.2515 | −1.3595 | −0.5345 | −0.2835 | −1.7685 | −1.2155 | −2.5325 | −1.2455 | −1.1785 | −1.5475 | −1.8225 | 0.0365 | −2.5475 |
| SLC6A4 | −3.1395 | 2.5915 | −3.1925 | −1.9605 | −2.0685 | 0.5825 | 1.6115 | −0.1825 | 1.2645 | 3.2735 | 0.9745 | −4.0355 | −3.5015 | −3.0775 | −1.8585 |
| SLC9A3R1 | −1.8645 | −0.4715 | −1.5895 | −0.0875 | −0.3445 | −0.8385 | −0.0685 | −0.9205 | −1.1205 | 0.5315 | −0.1985 | −0.8195 | −2.0855 | −1.5495 | −1.2765 |
| SLITRK6 | 0.82 | −0.607 | −0.433 | 0.028 | 0.447 | −0.105 | 0.234 | 0.851 | 0.268 | 0.565 | −0.579 | −0.229 | 1.376 | 1.835 | −0.073 |
| SMA3 | −0.133 | 1.287 | −0.038 | 0.951 | −1.483 | 0.277 | 1.569 | 1.058 | 0.908 | 1.199 | 1.526 | 1.148 | 1.241 | 0.477 | 1.252 |
| SMAD2 | 0.81 | −0.74 | 0.024 | −0.157 | 0.767 | 0.744 | −0.339 | 0.019 | 0.061 | −0.154 | −0.087 | 0.802 | 0.77 | 1.94 | 0.05 |
| SMO | −0.289 | −0.333 | −0.94 | 0.391 | −0.565 | −0.283 | −0.084 | −0.071 | 0.163 | −0.258 | −0.749 | −0.007 | 0.503 | 0.012 | 0.137 |
| SMU1 | 0.485 | −0.392 | −0.299 | 0.022 | −0.146 | 0.588 | 0.504 | −0.335 | 0.346 | −0.235 | 0.249 | 0.273 | 0.817 | −0.036 | −0.343 |
| SNPH | 0.396 | 0.831 | 0.365 | 0.304 | 0.649 | 0.663 | −0.147 | 0.589 | 0.621 | −0.142 | 0.928 | 0.243 | 0.201 | 0.441 | 0.77 |
| SNRPB2 | −1.0295 | −1.1425 | 0.5765 | 0.3585 | 0.8975 | 0.8635 | 0.2815 | 0.2885 | 0.4085 | −0.6675 | 0.2595 | 0.7155 | 0.2385 | −0.2995 | −0.2116 |
| SNRPD1 | 1.088 | 0.571 | 0.992 | 1.293 | 0.781 | 0.072 | −0.164 | −0.197 | 0.037 | −0.39 | 0.592 | 0.183 | −0.03 | 0.502 | 0.287 |
| SNRPD3 | 1.2765 | 0.1375 | 0.3025 | −1.0575 | 0.7035 | −0.9975 | −1.3005 | 1.0935 | 0.7315 | 0.8345 | 0.4215 | 0.9715 | 0.2105 | 0.2805 | 1.1645 |
| SNRPG | 2.0025 | 1.0285 | 1.3495 | 2.0445 | 1.9615 | 0.3625 | 1.5015 | 3.1265 | 1.2835 | −0.7555 | 1.5255 | 1.1895 | 2.7335 | 3.8375 | 1.0475 |
| SNX7 | −0.0945 | −0.0245 | 0.3215 | 0.5425 | 0.0015 | −0.6165 | −0.3075 | −0.1985 | 0.1775 | 0.8125 | −0.2875 | −0.3555 | −0.8515 | −0.9925 | 0.0435 |
| SO02 | 0.0025 | 0.9515 | −0.6575 | 1.0365 | 0.1775 | 0.3735 | 0.2075 | −0.0075 | 1.6005 | 1.0585 | 1.8635 | 0.8535 | −0.3685 | 0.3055 | 1.1175 |
| SORCS1 | 2.494 | −0.172 | 0.505 | 0.449 | 0.996 | −0.108 | 0.754 | 0.955 | −0.102 | 1.804 | −0.444 | 1.02 | −0.064 | 1.094 | 1.289 |
| SOX4 | −0.9715 | 1.1365 | −0.0885 | −1.0505 | −0.3245 | 0.4055 | −0.5025 | 0.5085 | 0.3585 | 0.0155 | 0.3875 | 0.9845 | −1.3505 | 0.3405 | 0.9315 |
| SOX9 | 0.214 | −0.7 | 0.21 | −0.09 | 0.462 | −0.292 | −0.207 | 0.058 | 0.233 | 0.05 | 0.177 | −0.248 | 0.458 | 0.545 | 0.813 |
| SPAG18 | −0.5355 | 0.5445 | −0.6685 | 0.4005 | −0.4215 | 0.6515 | 0.2675 | 0.2715 | 0.2845 | −0.1085 | 0.1375 | 0.8405 | 0.6135 | 0.3105 | −0.0325 |
| SPAG5 | −0.1915 | −1.4075 | 0.0105 | −0.9425 | −1.0975 | 0.0245 | −0.3235 | 0.1605 | −0.5825 | −0.3785 | 0.1525 | −0.8125 | −0.9785 | 0.7585 | −0.2505 |
| SPC18 | 0.781 | 0.285 | −1.158 | −0.226 | 0.267 | 0.731 | 0.421 | −1.044 | 0.306 | 0.394 | 1.099 | −0.61 | −0.714 | 0.282 | −0.045 |
| SPFH2 | 0.627 | −0.393 | −0.333 | 0.013 | 0.205 | 0.081 | −0.213 | −0.16 | −0.229 | 1.316 | 0.52 | 0.406 | 0.102 | 0.287 | 0.072 |
| SPG3A | −0.326 | 1.435 | 1.885 | 1.395 | 1.515 | 3.01 | 2.625 | 1.145 | 0.08 | 0.485 | −0.115 | 1.835 | 1.046 | −0.644 | 1.139 |
| SPPL3 | −1.599 | −0.353 | −0.374 | 0.19 | −1.433 | −0.728 | −0.528 | 0.108 | −0.867 | −1.903 | −0.761 | 2.022 | −0.15 | −0.241 | −0.342 |
| SQLE | −1.321 | 0.559 | −0.012 | −0.053 | 0.432 | −0.988 | −1.081 | −0.71 | −2.012 | −1.054 | −0.451 | −3.311 | −1.58 | 1.405 | −1.744 |
| SQRDL | 0.3685 | 0.9415 | 0.2785 | 0.1465 | −1.4385 | 0.3775 | −0.4285 | −0.3725 | 0.6895 | −0.1135 | −0.1015 | 0.5045 | 0.1365 | 1.1595 | 1.8395 |
| SREBF1 | 0.5925 | 0.3115 | 0.6185 | 0.2795 | 0.5445 | 0.4835 | 1.0285 | 1.9525 | 1.3405 | 0.7015 | 1.5565 | 1.7415 | 1.0295 | 2.1105 | 1.8735 |
| SRI | 0.6185 | 0.5855 | −0.2065 | 0.7095 | −0.0985 | 0.0595 | 0.0485 | 0.1775 | 0.5285 | −0.1205 | 1.0755 | 0.6485 | −0.5445 | 1.9885 | 0.4585 |
| SRPK1 | 0.1305 | −0.0815 | −0.9755 | −0.7315 | 0.3965 | −0.2815 | −0.3745 | −0.3545 | −0.1175 | 0.0395 | 0.0945 | 0.0725 | −0.3915 | 0.2795 | −0.1415 |
| SSFA2 | −0.8765 | 0.3745 | 0.5825 | −0.5245 | −0.7705 | 0.0855 | −0.5975 | −0.0265 | 0.3665 | −0.2515 | −0.3285 | 1.0445 | −0.0855 | 0.4925 | 0.1025 |
| SSR4 | 0.2885 | −0.0815 | 0.4695 | 0.3055 | −0.5335 | 0.4745 | −0.6485 | −0.4045 | 0.3715 | −0.1615 | 0.4715 | −0.7385 | −0.3285 | −0.5545 | 0.2925 |
| STARD3NI | 2.1065 | 0.9605 | 0.3385 | 0.7985 | 0.0135 | 0.1705 | 0.1805 | 1.0805 | −0.4045 | 0.8555 | 1.7445 | 0.9205 | 0.0245 | 0.8025 | 0.2915 |
| STARD7 | −2.0805 | −1.3205 | −0.8535 | −1.0495 | −0.0585 | 0.0195 | −1.6855 | −0.7145 | −1.4235 | −1.2055 | −1.4675 | −0.6175 | −0.2475 | −2.0775 | 0.0315 |
| STAT1 | 0.7205 | 0.9535 | 0.9635 | −0.2415 | −0.2995 | 0.1205 | −0.2965 | 0.2125 | −0.3385 | 0.6995 | 1.2155 | 1.0965 | −0.0195 | 0.1095 | 1.1525 |
| STC2 | 0.5555 | 0.2385 | 0.7565 | 0.0615 | 1.8785 | 0.9335 | 0.2015 | 0.5655 | −0.1155 | 0.2575 | 0.3555 | 0.6035 | 0.1755 | 0.6815 | −0.2105 |
| STK24 | 1.3155 | 0.0905 | 0.4285 | 1.0415 | 0.9005 | 2.1325 | 0.7885 | 0.3835 | 1.0415 | 1.2505 | 2.2405 | 1.1055 | 1.1965 | 0.8865 | 2.3495 |
| STK6 | −0.382 | 0.24 | −0.125 | −0.488 | 0.858 | −0.787 | −0.131 | 0.027 | 0.426 | 0.282 | −0.174 | −0.453 | 0.362 | −0.118 | 0.273 |
| STMN1 | −0.7045 | −0.1755 | −0.3705 | −0.3985 | 1.0825 | −0.4865 | 0.5965 | 0.5575 | 0.1245 | −0.0525 | −0.7545 | 1.2405 | 0.7205 | 0.2525 | 0.2685 |
| STX1A | 0.6015 | 0.0725 | −0.4485 | −0.0945 | 0.5025 | 0.7195 | 1.0355 | 0.0575 | −0.7295 | −0.1585 | −0.4935 | 0.1245 | 1.1005 | 0.7295 | 0.3755 |
| STX3A | −1.0205 | 0.5175 | 0.4705 | 0.0025 | 0.7115 | 0.1715 | 0.1505 | 0.2255 | 0.1435 | 0.3035 | 0.1035 | 1.5285 | 0.3825 | 0.0845 | 0.6705 |
| STYX | 0.2065 | −1.7045 | −1.1945 | −1.8575 | −0.0055 | −0.0435 | 0.0155 | −0.4235 | −1.1575 | −0.3715 | −0.7485 | −0.2505 | −0.8016 | −0.4475 | −0.4845 |
| SUGT1 | 0.138 | −0.644 | −0.288 | −1.088 | −0.597 | 0.049 | 0.544 | 0.536 | 0.106 | 0.454 | 0.325 | 0.383 | −0.174 | 1.361 | 0.899 |
| SUMF1 | 0.6145 | 0.9875 | 1.2995 | 1.5215 | 2.0005 | 1.5805 | 1.0135 | 0.9735 | 0.6025 | 0.5895 | 1.1875 | 1.4005 | 2.4035 | 1.2705 | 0.8785 |
| SUMO2 | 0.0935 | −0.6435 | −0.6375 | −0.2895 | 0.1375 | 0.2265 | 0.9895 | −0.8435 | −1.0655 | −0.7925 | −0.3105 | 0.1285 | 0.1115 | 0.6595 | −0.0215 |
| SUV39H2 | 0.9065 | −2.4585 | −2.3045 | 0.0035 | 1.7985 | 0.6985 | −0.8595 | 0.1075 | −0.8205 | 0.7376 | −0.0145 | 0.3145 | −2.9255 | 0.2285 | 0.4465 |
| SYAP1 | 0.7295 | −0.2825 | 1.3335 | 1.3285 | 1.3425 | 1.6135 | 1.2985 | −0.4645 | 0.0835 | −0.4385 | 1.7115 | −0.3995 | −0.5985 | 1.6445 | −0.0715 |
| SYCP2 | −2.453 | −1.908 | −2.165 | −1.183 | −1.465 | −0.31 | −1.395 | −0.935 | −0.744 | −0.77 | −1.867 | −1.888 | −0.096 | −1.48 | −2.432 |
| SYNGR1 | −1.0955 | −0.0235 | 0.8435 | −0.7805 | −0.2655 | 0.0315 | 1.1525 | 0.2585 | 0.2555 | −0.4435 | 0.5065 | −0.5805 | −0.0455 | −0.1265 | −0.5776 |
| SYTL2 | 0.0065 | 0.1255 | −0.0505 | −0.3355 | 0.8546 | 0.0315 | 0.8595 | 0.5115 | 0.2895 | 0.4705 | 0.0595 | 0.2365 | 0.5385 | 0.3715 | 0.2185 |
| TAF18 | 0.4025 | 0.3505 | 0.4855 | 0.0215 | 0.6325 | −0.2495 | 1.0405 | 0.1565 | 0.4945 | 0.8476 | 1.3325 | 0.9395 | −0.5505 | 0.8615 | −0.3335 |
| TAF8 | 0.342 | −0.004 | −0.139 | 0.264 | 0.697 | −0.305 | 0.712 | 1.388 | 0.537 | −0.018 | 0.948 | 1.11 | 0.03 | −0.096 | 0.349 |
| TARBP1 | 0.308 | 1.884 | 1.25 | 0.806 | 1.013 | 1.605 | 1.9 | 1.182 | −0.035 | 0.522 | 0.458 | 1.325 | 0.805 | 0.305 | 1.125 |
| TARS | −0.0035 | −0.5975 | −0.7425 | −0.3995 | −0.0075 | −0.0405 | 0.4085 | −0.0015 | −0.4825 | −0.3545 | 0.4485 | 0.1805 | −0.9825 | 0.0375 | −1.0935 |
| TATDN1 | −1.2885 | −0.7185 | −1.0715 | −0.6525 | −0.9105 | −0.1645 | 0.5145 | 0.3655 | −0.7355 | −1.6305 | −0.8215 | 0.5475 | −0.1125 | 0.6975 | −0.2355 |
| TBC1D12 | 0.2455 | −0.0575 | −0.2995 | −0.7285 | −0.8385 | −1.8065 | −0.7805 | −0.2725 | −0.1085 | 0.1175 | −0.0845 | −0.4545 | −0.2245 | 0.0355 | −0.3705 |
| TBC1D14 | −0.324 | 0.127 | −0.38 | 0.248 | −0.441 | 0.584 | −0.242 | −0.289 | −0.313 | 0.004 | −0.347 | 0.378 | 0.137 | 0.312 | 0.477 |
| TBC1D3 | −1.8145 | −0.4605 | −0.2035 | −0.0885 | 1.4245 | 0.1705 | 2.1185 | −0.5155 | 0.5755 | −0.1105 | 0.8285 | −1.2335 | 0.5085 | −1.7245 | 1.5885 |
| TBCA | −2.224 | −4.019 | −0.847 | −1.594 | −2.552 | 0.165 | −1.501 | −1.244 | −0.943 | 0.468 | 0.45 | −2.596 | −2.583 | −3.135 | −3.309 |
| TBX1 | −0.3526 | −0.4235 | −0.0785 | −0.0135 | −0.0355 | 0.0435 | −0.0235 | −0.5425 | 0.2595 | 0.0465 | −0.3245 | −0.5195 | 0.1745 | −0.2795 | −0.1645 |
| TBX3 | −0.727 | −1.317 | −1.831 | −0.833 | −0.548 | −0.312 | 0.187 | −0.752 | −0.529 | −0.362 | −1.167 | 0.298 | −0.733 | −0.878 | −0.858 |
| TCAP | −0.255 | −1.252 | −1.37 | −1.69 | −0.093 | −0.189 | −0.274 | −0.383 | −1.389 | −0.949 | −0.771 | −0.207 | −0.511 | −1.058 | −0.746 |
| TCEAL1 | −0.3435 | −1.2995 | −1.3806 | −1.2135 | 0.0305 | 0.4225 | −0.1615 | −1.2495 | −0.3845 | −0.7425 | −0.9395 | −0.1615 | −0.4705 | −0.7565 | −0.8105 |
| TCEAL3 | 0.256 | 2.734 | 1.006 | −0.753 | 1.06 | 0.605 | 1.336 | −0.996 | 0.49 | −0.83 | −1.307 | 0.04 | 0.169 | 1.202 | −0.967 |
| TCEAL4 | 0 | −0.639 | −0.15 | 0.538 | 1.627 | 0.327 | −0.058 | 0.091 | 1.052 | 0.279 | 0.463 | −0.478 | 0.136 | 0.206 | 0.527 |
| TCF15 | 1.238 | 0.373 | 0.813 | −0.625 | 0.248 | 0.492 | 0.128 | 0.327 | 0.963 | 1.048 | 1.765 | −0.18 | −0.114 | 0.11 | 0.37 |
| TCF3 | 0.064 | 0.832 | 0.249 | −0.813 | −0.318 | −0.262 | −0.762 | 0.29 | −0.14 | −0.178 | −0.18 | 2.107 | 0.248 | −0.012 | 0.719 |
| TCF7L2 | −0.2225 | −0.6095 | −0.7635 | −0.7265 | −0.1035 | −0.3835 | −0.3285 | −0.4015 | −0.5845 | −0.2985 | −0.2445 | −0.2305 | −0.2185 | −0.4305 | 0.2485 |
| TDRD7 | −4.3155 | −4.4315 | −3.9685 | −0.2845 | −3.8195 | −0.5105 | −3.8535 | −4.5085 | −2.4595 | −2.8385 | −2.4855 | −4.4115 | −3.9885 | −3.6375 | −4.2115 |
| TESK2 | 2.621 | 2.941 | 3.457 | 2.668 | 2.238 | 1.831 | 1.218 | 1.442 | 2.091 | 0.413 | 2.221 | 0.546 | 0.917 | 1.847 | 0.748 |
| TFAP2B | −0.39 | 1.688 | 1.885 | −0.36 | 0.515 | 0.916 | 1.695 | 1.522 | 1.168 | 1.931 | 2.425 | 0.711 | 0.838 | 0.09 | 1.058 |
| TFCP2L1 | −1.8035 | −0.9015 | −1.1125 | −1.4095 | −1.0185 | −1.3865 | −1.3455 | −1.3485 | −1.3475 | −1.4365 | −1.4755 | −1.2595 | 0.5405 | −0.7425 | −1.2115 |
| TFCP2L2 | −3.7055 | −3.7325 | −4.3695 | −2.8925 | −3.9335 | −0.6025 | −3.7935 | −4.0045 | −3.8135 | −2.9795 | −4.8915 | −4.2145 | −4.5775 | −2.9715 | −4.2875 |
| TFF1 | −0.6665 | 0.3845 | 0.0585 | 1.9675 | −0.2645 | 0.5955 | 0.2265 | −0.3135 | −0.0395 | 1.7415 | 2.0975 | 2.2095 | 1.3835 | 0.0775 | 1.7125 |
| TFF3 | 0.748 | 2.281 | −0.486 | 0.019 | 0.058 | 1.871 | −0.353 | −0.744 | −0.281 | 1.327 | 0.338 | −1.048 | −0.973 | −0.589 | −0.479 |
| TFRC | −0.4895 | 0.4385 | −0.6645 | −0.2925 | 0.3655 | 0.4235 | 1.1835 | 0.1718 | 0.3085 | 0.0345 | −0.2825 | −1.2145 | −0.4615 | 0.0405 | 0.1425 |
| TGFBR3 | 0.405 | 0.967 | −0.473 | 0.414 | 0.743 | 0.551 | 0.458 | 0.307 | 0.01 | 0.316 | 0.058 | 0.883 | 0.039 | 0.304 | 0.07 |
| THY28 | −1.7475 | 0.6165 | −0.3765 | −0.2845 | −1.3045 | 0.1795 | −0.8605 | −0.0195 | 0.5845 | 0.3575 | −0.4605 | −1.1525 | −1.2815 | −1.3165 | −0.8365 |
| TIF1 | −0.882 | 0.27 | 0.374 | 0.48 | −0.311 | −0.629 | 0.769 | −0.503 | 0.007 | −0.499 | −0.249 | 0.52 | 0.274 | 0.073 | 0.472 |
| TIGA1 | −0.074 | −1.299 | −0.295 | −0.175 | 0.372 | 0.662 | −0.343 | −0.134 | 1.147 | 0.432 | 0.209 | −1.000 | −0.225 | 0.122 | 0.325 |
| TIM14 | 0.6175 | −0.6675 | −0.2315 | −0.3805 | −0.1125 | 0.1315 | 0.6085 | 0.0175 | −0.8015 | 0.6335 | −0.3955 | 0.2925 | 0.3725 | −0.0245 | −0.2375 |
| TIMELES [symbol] | −0.1735 | −0.0035 | −0.6765 | −0.0435 | −0.6975 | −0.1385 | −0.4425 | −0.4665 | 0.6485 | 0.9375 | −0.9525 | 0.1105 | −0.8195 | −0.6125 | −0.1115 |
| TIMM9 | 1.78 | −0.866 | 1.551 | 0.164 | 2.808 | 1.937 | 1.924 | −0.094 | 1.561 | 1.462 | 1.463 | 2.457 | 0.349 | 0.692 | 0.56 |
| TINP1 | 1.8555 | −0.0035 | 1.2075 | 0.5215 | 0.9015 | −0.0045 | 0.8195 | 0.7525 | 0.8055 | 0.5085 | 0.5325 | −0.5585 | −0.3885 | 1.0375 | 0.8395 |
| [symbol] IP1208 | −1.193 | −2.226 | 0.311 | −0.01 | −0.623 | 0.482 | 0.423 | 0.727 | −0.845 | −0.242 | −0.008 | −0.459 | −1.144 | −0.375 | −1.877 |
| TK1 | −0.085 | 0.745 | 1.871 | 0.97 | 1.012 | 0.402 | 1.287 | 0.109 | 0.687 | 0.368 | 1.691 | 0.783 | 0.029 | 0.985 | −0.224 |
| TLE2 | −0.237 | 0.037 | −1.438 | −0.020 | 1.764 | 1.64 | 2.308 | 1.919 | −0.027 | 1.953 | 0.03 | −0.226 | 0.089 | 1.68 | −0.271 |
| TLR5 | −0.584 | −1.506 | −1.925 | −1.047 | −1.426 | −0.022 | −0.275 | −1.191 | −1.086 | −1.275 | −0.869 | −1.316 | −0.853 | −0.527 | −1.341 |
| TM4SF12 | 0.806 | −0.133 | 0.64 | −1.045 | −0.567 | −0.363 | 1.033 | 0.612 | 0.479 | −0.135 | 0.021 | 1.143 | 0.908 | 0.798 | 0.167 |
| TM4SF13 | −0.848 | −1.96 | −2.258 | −3.797 | 0.077 | −0.66 | −0.194 | −1.065 | −0.397 | −1.93 | −2.254 | −1.155 | −0.434 | −0.368 | −0.584 |
| TMEM14A | −0.2345 | −0.3255 | −0.3755 | −0.1895 | 0.1365 | 0.4385 | −0.8785 | −0.6985 | −0.0825 | −0.2135 | −0.3855 | −0.4735 | 0.2985 | 1.3435 | 0.9195 |
| TMEM25 | 0.8485 | 2.8815 | 3.0455 | 1.7895 | 1.8625 | 2.8555 | 0.5095 | −0.0585 | 1.3695 | 2.0685 | 3.8745 | 1.1545 | 0.9265 | 1.7925 | 0.2005 |
| TMEM4 | 0.738 | −1.179 | −0.635 | −0.336 | 0.431 | 0.362 | 0.108 | 0.731 | 0.832 | 0.146 | 0.423 | 0.708 | 0.153 | 1.108 | −0.433 |
| TMPRSS5 | 0.499 | −0.918 | −0.46 | −1.156 | −1.312 | 2.62 | 2.536 | 0.844 | 1.04 | 2.498 | 2.042 | 0.493 | 1.61 | 1.18 | 2.59 |
| TMSB10 | −1.864 | −1.882 | 1.549 | 1.022 | −1.763 | −1.645 | −0.18 | 3.846 | 0.994 | −0.573 | 0.077 | −0.162 | 1.678 | 0.59 | 1.533 |
| TMSNB | 0.2875 | −0.1405 | −0.1415 | 0.2255 | 0.2255 | 0.6525 | 1.4435 | 0.5785 | 1.3175 | 0.3825 | 0.0085 | 0.8185 | 1.2145 | 0.4005 | 0.2855 |
| TNFRSF1 [symbol] | −0.029 | −0.215 | 0.187 | 0.519 | 0.838 | 0.981 | 1.489 | 0.612 | 1.713 | −0.543 | 0.188 | 0.424 | 1.413 | −0.118 | 0.111 |
| TNFRSF2 [symbol] | −0.8415 | −0.0455 | −0.4865 | 0.0065 | −0.2825 | −0.4745 | 0.5325 | 0.0945 | 0.3945 | −0.2445 | −0.4555 | 0.4785 | 0.7585 | −0.2405 | −0.2395 |
| TNFRSF2 [symbol] | −1.6235 | −0.7595 | 0.8135 | −0.5425 | 0.0985 | −0.8775 | 0.6705 | 1.3735 | −1.0345 | −1.0285 | −0.9145 | −1.2225 | −0.8255 | 0.1405 | −0.5865 |
| TNFSF4 | 0.4785 | −0.9285 | −0.5395 | −0.7295 | −0.7375 | −0.8376 | 0.0805 | −0.9495 | −0.6485 | −0.8515 | −0.8475 | −1.0535 | −1.0275 | −1.3055 | 0.0905 |
| TNFSF7 | 0.6905 | 0.9255 | −0.6415 | −0.1815 | 0.2165 | 0.0175 | −0.6335 | −0.7045 | 0.6235 | −0.7465 | −0.0305 | 2.3785 | −0.0075 | −0.2895 | 2.3165 |
| TNIK | 3.9595 | 3.2455 | −0.0405 | −0.4665 | −0.7825 | −0.2665 | 3.7385 | 2.1195 | 3.6475 | 2.4355 | 1.6685 | −1.0035 | −0.1865 | 2.6075 | −0.3215 |
| TNNC2 | 0.883 | −0.31 | 0.645 | −1.43 | 0.223 | 0.577 | −0.559 | −0.493 | −0.001 | 1.011 | −1.033 | −0.721 | 0.094 | 0.212 | 0.036 |
| TNNI2 | −0.218 | −0.606 | −1.06 | −0.965 | 0.39 | −0.452 | 0.65 | −1.898 | −1.379 | 0.261 | −0.52 | −2.067 | −1.3 | −0.348 | −0.421 |
| TNNI3 | −0.9275 | −0.5415 | −1.2235 | −1.8765 | −1.4735 | −0.9375 | −0.2195 | −0.5115 | −0.4355 | 1.5985 | 0.3235 | −0.2095 | −0.1485 | −0.0495 | 0.3305 |
| TOB1 | −0.0635 | 0.2465 | −0.6665 | −0.0835 | −0.6905 | 0.0175 | 0.2105 | −0.2325 | −0.4395 | −0.7075 | −0.3385 | −0.7405 | −0.3655 | 0.0985 | −0.6145 |
| TOM1L1 | 0.5605 | 1.4375 | 0.6435 | −0.8805 | 1.1995 | 1.4305 | 0.7185 | −0.4425 | 0.1935 | 0.0695 | 0.0515 | 0.9055 | −0.1305 | 0.2135 | 0.4445 |
| TOMM7 | 0.8955 | −0.0095 | 1.2415 | 0.7355 | 0.3245 | 1.0785 | 1.0035 | 1.1055 | 1.1675 | 0.5945 | 0.9495 | 1.2605 | −0.3405 | 0.6995 | 0.3315 |
| TOP1MT | −0.516 | −0.221 | −0.441 | 0.289 | 0.397 | −0.073 | 0.305 | −0.023 | 0.004 | −0.156 | 0.763 | 0.09 | −0.152 | −0.312 | −0.083 |
| TOP2A | 0.5725 | −0.1165 | 1.1725 | −0.2325 | 0.6335 | −0.0275 | −0.3345 | 0.4895 | 0.5475 | 0.5985 | 1.0555 | 0.1135 | −0.4135 | 0.2815 | −0.1145 |
| TOP2B | 1.697 | −0.305 | 1.668 | 1.559 | 1.421 | 2.246 | 1.981 | 1.045 | 0.263 | 1.596 | 1.338 | 2.324 | 0.247 | 1.798 | 2.07 |
| TOPBP1 | −0.9245 | −0.5035 | −1.8145 | −1.9775 | −1.9705 | −1.1045 | −0.2945 | −1.0045 | −1.1135 | −0.3795 | −1.2785 | −1.6285 | −0.9915 | −0.6715 | −0.3985 |
| TOPK | −0.0245 | 0.6755 | 0.8755 | 0.3865 | 1.5875 | 0.4895 | 2.8035 | 2.4255 | 0.6065 | 0.8295 | 0.8705 | 2.5585 | 1.0715 | 1.4675 | 0.8815 |
| TP53AP1 | −1.6635 | −0.9585 | −1.5495 | −0.7835 | −0.8455 | 0.1385 | −1.2155 | −0.5055 | −0.8845 | −1.0165 | −0.2335 | −1.1675 | −1.2935 | −1.2035 | −0.0795 |
| TP53BP2 | 0.5685 | 0.6615 | 0.4155 | 1.2525 | 0.8585 | 0.3095 | 0.1715 | −0.5845 | 0.1405 | 0.1695 | 0.8925 | 2.1835 | 2.8755 | 1.0435 | 0.3445 |
| TP53INP1 | 0.2115 | 0.8155 | 1.2765 | 0.9625 | 1.9755 | 1.5405 | 1.3595 | 1.2615 | −0.0075 | 0.5505 | 0.6265 | 1.5365 | 0.3445 | 1.7335 | 0.9605 |
| TPD52L1 | −0.304 | 0.084 | 1.342 | 1.549 | −0.238 | 0.474 | −0.805 | −0.476 | −0.054 | −0.405 | 1.406 | 0.107 | 0.236 | −0.175 | 0.892 |
| TPRT | −0.7505 | −0.2895 | 0.1175 | −0.1205 | −0.2015 | −0.4845 | −0.5925 | −0.4005 | 0.2745 | 0.3575 | 0.0915 | −1.0905 | −0.8315 | −0.1805 | 0.3385 |
| TRA@ | −1.604 | −0.525 | −1.036 | −0.09 | −0.015 | 0.118 | 0.698 | 0.078 | 0.036 | −0.083 | −0.619 | −0.596 | −0.239 | −0.435 | −0.289 |
| TRAF4 | −0.001 | 0.348 | 0.459 | 0.559 | −0.088 | −0.456 | −0.383 | −0.538 | 0.379 | −0.293 | −0.2 | −0.396 | 0.05 | −0.34 | −0.192 |
| TRAPPC4 | 0.045 | 3.307 | 1.016 | 2.919 | 0.092 | 0.181 | −0.006 | −0.042 | 0.919 | −0.342 | 1.347 | 0.087 | −0.25 | 0.197 | −0.313 |
| TRH | 0.8625 | 1.8235 | 2.5955 | 1.8945 | 1.0445 | 0.5675 | 2.7875 | 2.2475 | 1.5985 | 2.1075 | 2.6355 | 2.3175 | 0.8085 | 0.5205 | 1.9005 |
| TRIM17 | 0.4065 | 0.9995 | 0.3745 | 0.4245 | −1.0425 | −0.2175 | −0.2765 | −0.0445 | 0.0315 | −0.7195 | −0.2805 | 1.2925 | −0.1255 | −0.2725 | 0.4245 |
| TRIM2 | −0.8255 | −0.7645 | −0.5285 | −0.1805 | −0.5735 | −0.0975 | −0.6165 | −0.4215 | 0.2925 | 0.1845 | −0.4955 | 0.1095 | 0.0185 | 0.2185 | 0.1055 |
| TRIM6 | −0.548 | 0.475 | 0.137 | −0.389 | 0 | −0.106 | 0.342 | 0.475 | −1.229 | 0.114 | 0.424 | −0.19 | −0.712 | −1.212 | −0.834 |
| TRIP3 | 0.2345 | 0.6095 | 0.0575 | 0.7125 | −0.3055 | −0.1655 | −0.3955 | −0.0465 | 0.3375 | −0.0725 | −0.3825 | 0.0035 | 0.2385 | 0.4195 | 0.0995 |
| TRPS1 | −1.2145 | −0.0215 | −0.2525 | −0.3445 | −0.4135 | −0.1275 | −0.2035 | −0.4495 | −0.2085 | 0.0276 | 0.5675 | −1.0485 | −1.0245 | 0.0495 | −0.3965 |
| TSG101 | −0.6205 | −1.5455 | −0.2085 | −0.8085 | −1.0845 | 0.0885 | −0.4235 | −1.2925 | −0.0155 | 0.1555 | −0.7825 | −0.8145 | −1.6335 | 0.5605 | −1.9175 |
| TTC10 | 1.8795 | 0.3325 | 2.4885 | 1.5465 | 1.8135 | 1.6475 | 1.4865 | 1.4015 | 1.8805 | 1.7685 | 2.6755 | 0.7985 | 0.4915 | 1.3505 | 1.6815 |
| TTC12 | −0.376 | 0.284 | 1.80 | 1.218 | 0.225 | −0.173 | 0.869 | 0.921 | 0.166 | 0.606 | 1.475 | 1.796 | 0.703 | 0.461 | 0.912 |
| TTK | 0.9345 | 3.7275 | 0.8485 | 3.4875 | 0.3695 | 4.5545 | 2.0195 | 3.5445 | 0.8585 | 3.7465 | 5.3315 | 2.4725 | 0.4185 | 2.5565 | 0.4505 |
| TTLL4 | 0.302 | 0.136 | −0.93 | 0.814 | 0.953 | 0.335 | 1.257 | 1.729 | 1.002 | −0.352 | −0.717 | −0.675 | 1.309 | 1.344 | 0.527 |
| TTYH1 | −0.7705 | 0.0405 | 0.3285 | −0.2985 | −0.1235 | −0.0575 | 0.3885 | 0.4885 | 0.8355 | 0.5805 | 0.2975 | −0.8155 | −0.4755 | −0.0085 | 1.0025 |
| TUBB3 | −0.4785 | 0.9155 | 1.0625 | 0.8565 | 0.6605 | 0.5115 | −0.0265 | −0.3045 | −0.0125 | 0.2075 | −0.1995 | 1.8305 | 0.4195 | 0.7705 | 0.4335 |
| TULP3 | 1.695 | −0.245 | 0.905 | 0.525 | 1.505 | 1.185 | 1.775 | 1.475 | 0.111 | 0.885 | 2.285 | 1.115 | 0.109 | −0.27 | 1.671 |
| TXN | 0.084 | −0.155 | 0.257 | −0.221 | 0.111 | 0.251 | −0.069 | −0.116 | 0.224 | 0.16 | 0.885 | −0.567 | 0.119 | 0.335 | −0.015 |
| TYMS | 0.8505 | 0.5995 | −0.1455 | −0.0495 | 0.8505 | 0.7875 | 1.5805 | 1.2705 | 1.3235 | 1.0455 | 0.9385 | 1.7345 | 0.9385 | 0.8245 | 1.3835 |
| U5-116KD | 0.1835 | 0.0805 | −0.4525 | −0.2265 | 0.5805 | 0.0695 | 0.1295 | −0.8405 | −0.6885 | −0.7155 | −0.1155 | −0.5355 | 0.2775 | 0.6085 | 0.0195 |
| UBE2E3 | −0.744 | −0.294 | −0.074 | −0.476 | 0.383 | 0.087 | −0.663 | −0.15 | −0.594 | −0.303 | 0.518 | −0.442 | −0.434 | −0.108 | −0.078 |
| UBL5 | 0.3905 | −0.8445 | −0.9775 | −0.4655 | −0.7605 | 0.1345 | 0.0795 | 3.3425 | 3.5975 | 0.5285 | 1.4905 | 3.7445 | 2.5875 | 1.8275 | 4.3375 |
| UBN1 | 0.218 | 0.72 | 2.549 | 1.766 | 1.33 | 1.612 | −0.230 | 0.251 | 0.498 | −0.038 | 0.983 | 1.846 | 0.094 | 1.05 | 0.422 |
| UCHL1 | −1.286 | 0.008 | 1.565 | 0.121 | −0.851 | 0.148 | −1.085 | −0.831 | −0.761 | −0.866 | −1.008 | −0.856 | −1.485 | −1.259 | −0.393 |
| UCHL3 | 1.269 | −1.009 | 0.144 | 0.487 | 0.909 | 0.372 | 0.096 | 0.281 | 0.049 | −0.097 | −0.025 | 0.353 | 0.582 | 1.002 | 0.313 |
| UGCG | −0.9465 | 0.5135 | 0.3375 | −0.0665 | −0.1905 | −0.9715 | 0.1465 | −0.2135 | 0.2845 | 0.0815 | −0.5305 | −0.6215 | −0.2605 | −0.8195 | −0.1925 |
| UGCGL1 | 1.5175 | 1.6105 | 2.4455 | 0.3245 | −0.2015 | −0.5885 | −0.2725 | 1.6115 | 1.3785 | 0.4405 | 0.3745 | 0.5945 | 1.3525 | 1.6485 | 0.3075 |
| UGT2B7 | 0.5345 | −0.5845 | −0.3665 | 0.0375 | 0.2105 | −0.0605 | −0.7545 | 0.4685 | −0.4545 | −0.8795 | −0.2265 | 0.3065 | −0.3925 | 0.2325 | −1.2235 |
| UNC13D | 0.017 | −0.894 | −0.58 | −0.839 | −0.578 | 0.072 | −0.1 | −0.546 | −1.008 | 0.294 | 0.347 | 0.744 | −0.117 | 0.301 | 0.593 |
| VAMP8 | −0.568 | −0.212 | 0.221 | −0.111 | −0.423 | 0.28 | −0.937 | 0.045 | 0.202 | −0.459 | 0.368 | −0.348 | −0.347 | −0.407 | −0.118 |
| VAPA | −2.8345 | 0.1575 | −2.2435 | −2.1965 | −0.4545 | −0.6415 | −0.9465 | −1.5855 | −2.6545 | −3.3825 | −4.7855 | −2.6815 | −2.8625 | −1.7295 | 1.5065 |
| VAV1 | −0.649 | 0.386 | −1.563 | −1.696 | 0.734 | −0.923 | −0.636 | −0.521 | 0.051 | 0.376 | −0.023 | −1.529 | 7.391 | 0.025 | 0.333 |
| VAV3 | 0.503 | −0.508 | −0.27 | 0.251 | −0.271 | 0.307 | 0.195 | 0.677 | 0.273 | 0.148 | 0.391 | 0.352 | −0.047 | 1.507 | −0.291 |
| VCX3 | −0.42 | 0.04 | −0.608 | 0.102 | 0.126 | −0.185 | −0.541 | −0.308 | 0.504 | −0.002 | −0.418 | −0.596 | 0.019 | −0.198 | −0.189 |
| VDAC2 | 1.1085 | 2.6485 | 2.3085 | 1.1885 | 0.3005 | 2.6315 | 2.5885 | 2.5985 | 2.1175 | 1.1885 | 2.7185 | 1.8185 | 1.2645 | 0.9595 | −1.4745 |
| VEST1 | −0.054 | 0.037 | −0.007 | 0.039 | 0.291 | 0.022 | −0.288 | −0.093 | 0.021 | 0.94 | 0.273 | −0.467 | −0.278 | −0.165 | 0.082 |
| VGLL1 | 1.8165 | 2.2805 | 2.8615 | 2.2225 | 2.5765 | 1.7775 | 0.1665 | −0.5735 | 0.5225 | 0.5305 | 2.4415 | −0.6075 | −0.2575 | −0.2905 | 0.3455 |
| VMD2L2 | 1.019 | −0.161 | −0.687 | 0.113 | 0.094 | 0.213 | 0.728 | 0.036 | 0.082 | −0.584 | −0.302 | 0.294 | 1.628 | −0.131 | −0.818 |
| VSX1 | 0.033 | −0.192 | −0.539 | −0.232 | −0.073 | −0.514 | 0.846 | 0.715 | −0.123 | 0.1 | 0.209 | 0.877 | −0.301 | 0.857 | −0.538 |
| WARP | −0.751 | −1.485 | −0.781 | −1.239 | 0.101 | −0.077 | 0.205 | −0.41 | −0.427 | −0.772 | −0.352 | −0.642 | 0.232 | 0.068 | −0.147 |
| WDR26 | −0.0025 | −0.8525 | 1.6935 | 0.1065 | −0.6985 | −0.5345 | −0.2845 | 0.4335 | −0.3365 | 0.1665 | 0.3685 | −0.6595 | −0.4985 | −0.2855 | 0.2405 |
| WDR6 | −0.95 | 0.547 | 0.034 | 1.471 | 0.366 | −0.567 | −0.522 | −0.455 | 0.146 | −0.577 | 0.481 | −0.358 | −0.111 | 0.575 | −0.408 |
| WHSC1L1 | −0.589 | 0.131 | −0.339 | 0.701 | 1.141 | 1.563 | −0.329 | 3.851 | 0.921 | 2.091 | 0.041 | 1.241 | 1.436 | 2.975 | 0.009 |
| WNT3 | 0.0355 | −0.8655 | −0.3585 | −0.2425 | −0.6775 | 0.1895 | 0.0115 | 0.3695 | 0.1035 | 0.1535 | 0.8995 | 0.5595 | 0.1335 | 0.3955 | 1.4655 |
| WNT6 | −1.2345 | −1.5565 | −0.7705 | −1.2285 | −0.4135 | −0.2255 | −1.1825 | −0.7805 | −1.0925 | −0.9855 | −0.9275 | −0.1265 | −1.2425 | −1.1715 | −0.8275 |
| WSB2 | 1.2135 | 1.8725 | 0.1135 | 0.1575 | −0.2755 | 0.0295 | 2.4645 | 2.1985 | 0.2625 | 1.2955 | 1.6435 | −0.2135 | −0.0575 | −0.6685 | −0.1275 |
| WWP1 | −3.1335 | −3.9555 | −3.0785 | −2.5805 | −2.3435 | −2.3505 | −2.3165 | −2.3425 | −3.3585 | −3.2555 | −2.6275 | −2.3075 | −1.8065 | −2.7895 | −2.8975 |
| WWP2 | −1.074 | −0.941 | −1.288 | −1.848 | −0.514 | −1.101 | 0.6 | −0.867 | 0.159 | 0.399 | −0.453 | −1.158 | −0.325 | −0.129 | 0.506 |
| XBP1 | −0.044 | 0.004 | −0.653 | 0.298 | 0.42 | 0.958 | 0.198 | 0.227 | −0.606 | 0.13 | 0.119 | −0.597 | −0.215 | 0.018 | 0.326 |
| XYLT2 | 0.7995 | 0.6655 | 0.1275 | 0.3825 | 0.7425 | −0.1095 | 0.6135 | −0.1245 | 0.1065 | 0.3125 | 0.3595 | 0.2955 | 0.4535 | 1.1555 | 0.3665 |
| YBX2 | −0.435 | −0.578 | −0.619 | 0 | −1.374 | 0.341 | −0.175 | −0.293 | 0.156 | −0.259 | −0.358 | 0.565 | 0.012 | −0.05 | 0.503 |
| YIF1 | −1.2285 | −1.3285 | −0.1385 | −0.8715 | −1.4855 | 0.8295 | −0.5975 | −1.3655 | −0.6895 | −0.7205 | −1.5535 | −1.2165 | −0.9955 | −1.1435 | −0.6885 |
| ZA20D3 | 0.8945 | 1.8165 | 0.9895 | 0.3625 | 0.7745 | −0.1175 | 0.0015 | −0.3945 | 0.1695 | 0.6785 | 0.6075 | −0.0385 | −0.0145 | 0.9605 | 0.8325 |
| ZAP128 | −0.8275 | −0.1215 | −0.8355 | −0.5495 | −0.4875 | 0.0955 | −0.3065 | 0.3785 | 0.1765 | −0.0545 | −0.6605 | −1.1095 | −0.4095 | −0.5175 | −0.8285 |
| ZBED3 | −0.307 | −0.135 | −0.224 | −0.038 | 1.007 | 0.572 | 0.432 | 1.087 | 1.013 | 1.422 | 1.382 | −0.264 | 0.582 | 0.003 | 0.507 |
| ZBTB4 | −0.203 | −1.518 | −0.6 | −0.146 | 1.179 | 0.935 | 0.045 | −0.866 | 0.017 | −0.137 | 1.464 | −0.097 | 0.504 | −0.322 | 0.734 |
| ZCWCC1 | 1.8065 | 1.8535 | 1.8125 | 1.0255 | 0.0135 | 0.4225 | −0.0885 | 0.1765 | 0.6355 | 0.3505 | 1.0585 | 1.0875 | 1.5875 | 1.3795 | 0.8005 |
| ZCWCC2 | 0.277 | 1.046 | 1.264 | 1.792 | 1.232 | 0.929 | −0.081 | −0.426 | −0.204 | −1.291 | 0.854 | 0.006 | 0.371 | −0.394 | −0.214 |
| ZDHHC13 | −1.2005 | 0.4755 | −1.4895 | −0.4275 | −0.9145 | −0.0955 | 0.7165 | 0.7075 | 0.3705 | 1.9385 | 1.2205 | −1.2125 | 0.3025 | −0.3875 | −0.6275 |
| ZDHHC14 | 0.3565 | −0.9325 | −1.4045 | −0.5135 | −0.5195 | 0.4305 | −0.1045 | −0.6715 | 0.0055 | 0.3715 | −1.1335 | −1.0915 | 0.0945 | −0.5875 | −0.3076 |
| ZFHX4 | 0.7235 | −0.2215 | −0.3895 | −0.0255 | −0.1115 | 0.5485 | −0.0355 | −0.1445 | 1.8725 | 0.8355 | 0.5745 | −0.8385 | −0.1715 | −0.0095 | −0.0885 |
| ZFYVE21 | 0.631 | −0.182 | −1.156 | −2.556 | 1.579 | −1.488 | 1.041 | 0.746 | 0.43 | −0.982 | 0.169 | 0.27 | −2.017 | 0.686 | 0.418 |
| ZNF133 | −0.189 | −0.044 | −0.366 | −0.249 | −0.785 | −0.653 | −0.998 | −0.117 | −0.268 | −0.127 | −0.374 | −0.909 | −0.6 | −0.911 | −0.34 |
| ZNF165 | −0.5815 | −0.0485 | 0.0395 | 0.4345 | 0.2505 | −0.3945 | 0.7695 | 1.0715 | −0.5095 | −0.4295 | 0.5505 | 1.2925 | −0.3965 | −0.1035 | 0.8985 |
| ZNF24 | 0.377 | −0.114 | 0.061 | 0.413 | 0.808 | 0.255 | 0.819 | 0.722 | 0.368 | −0.42 | 0.107 | 0.503 | −0.688 | −0.139 | −0.396 |
| ZNF318 | 0.4635 | −0.0645 | −0.8055 | −0.3365 | 0.5375 | 0.2045 | 0.1935 | −0.0305 | 0.1875 | −0.2245 | −0.0285 | 0.2125 | 0.2935 | −0.1505 | 0.3035 |
| ZNF54 | 0.9625 | 2.1405 | 1.4865 | 0.8855 | 0.8475 | 1.2235 | 1.3105 | 1.4215 | 1.3306 | 1.9685 | 2.8645 | 0.8735 | −0.0335 | 0.2325 | 0.8315 |
| ZNF432 | 0.2275 | −0.4165 | −0.3845 | 0.7295 | 0.0495 | 0.5685 | 0.8045 | 0.2655 | −0.4915 | 0.4785 | −0.2765 | −0.0465 | 0.8745 | 0.2295 | 2.5265 |
| ZNF482 | −0.017 | −1.12 | −1.221 | −0.581 | −0.5 | −1.168 | −0.132 | −0.421 | −0.981 | −1.277 | −0.89 | −1.089 | −1.258 | −0.911 | −1.05 |
| ZNF505 | −0.524 | −0.061 | −0.973 | −0.41 | 0.546 | −0.174 | −0.295 | −0.11 | −0.184 | −0.65 | −0.535 | −0.774 | −0.242 | −0.956 | −0.108 |
| ZNF552 | −0.3315 | 0.0985 | −0.6295 | −0.3285 | −0.1035 | −0.0855 | −0.3825 | −0.2745 | 0.4255 | −0.2135 | 0.2265 | −1.0685 | −1.0225 | −0.5835 | −0.0245 |
| ZNF577 | −0.0225 | 0.1685 | 0.4115 | 0.0095 | 0.3055 | −0.1185 | −0.3955 | 0.1605 | 0.3965 | −0.0935 | 0.4555 | 0.5225 | 1.1875 | 0.6865 | 0.1785 |
| ZNF587 | 0.5345 | 0.2795 | −1.0245 | 0.4885 | 0.8295 | 0.7735 | 0.0375 | −0.1245 | −1.2205 | −0.2705 | 0.2545 | −0.7295 | 0.2205 | −0.6395 | 0.2405 |
| ZNF598 | −0.0275 | −0.3465 | −0.7325 | −0.9285 | −0.2875 | 0.4245 | 0.1965 | 0.1285 | −0.2025 | −0.0495 | 0.9015 | −0.4125 | −0.8335 | −0.1535 | 0.1955 |
| ZNF599 | 0.381 | −0.822 | −0.522 | −0.271 | 0.263 | 0.368 | 0.054 | 0.034 | 0.15 | 0.143 | −0.551 | −0.145 | −0.771 | −0.289 | 0.44 |
| ZNF606 | 0.559 | −0.322 | 1.173 | 0.578 | 0 | 0.316 | 1.883 | 0.241 | 1.024 | 0.437 | 0.803 | 0.999 | 0.738 | 0.578 | 1.997 |
| ZNF614 | 0.564 | 0.711 | 0.396 | −0.848 | 1.059 | 1.038 | 0.959 | 0.04 | −0.068 | −0.163 | −0.007 | 0.413 | −0.152 | 0.229 | 0.578 |
| ZNF639 | | | | | | | | | | | | | | | |
| ZNF7 | | | | | | | | | | | | | | | |

| | Sample | | | | | | | | | | | | | | |
|---|---|---|---|---|---|---|---|---|---|---|---|---|---|---|---|
| | BR02-703 [symbol] | BC00064 | BC00029T | PB257 | BR03-703 [symbol] | UB-100 | PB341-LN | BR98-026 [symbol] | BC00033- [symbol] | BC00093- [symbol] | BC00017- [symbol] | Normal Br | Normal Br | Normal Br | Normal Br |
| | Class | | | | | | | | | | | | | | |
| Symbol | BASAL | BASAL | BASAL | BASAL | BASAL | BASAL | BASAL | BASAL | NORMAL- [symbol] | NORMAL- [symbol] | NORMAL- [symbol] | NORMAL- [symbol] | NORMAL- [symbol] | NORMAL- [symbol] | NORMAL- [symbol] |
| | −1.3565 | −1.4885 | −1.3575 | −0.8005 | −1.1895 | −1.3045 | −1.8075 | −1.0205 | −0.2155 | −0.0145 | −0.6165 | −0.5255 | −0.5355 | 0.2885 | −1.3795 |
| | −0.945 | −0.277 | 0.43 | −0.322 | −0.139 | 0.734 | 0.127 | 0.012 | −0.378 | −0.448 | −0.04 | −0.126 | −1.059 | −0.341 | −0.123 |
| ABAT | −3.12 | −2.399 | −2.69 | −2.511 | −3.06 | −1.916 | −3.076 | −1.792 | −0.457 | −2.188 | −0.477 | −0.013 | −0.147 | 0.725 | −2.382 |
| ABC1 | −1.7285 | −0.5795 | 0.1315 | −0.0085 | −0.5535 | −0.5155 | −0.5955 | 0.2645 | −0.2875 | −0.9125 | 0.3585 | −1.0295 | −0.0645 | 0.6615 | −0.2705 |
| ABCC11 | 0.0115 | −0.4435 | −0.7085 | −0.8985 | −0.2145 | 0.3775 | −1.3355 | 0.9595 | −0.6265 | −0.4315 | 0.0265 | −0.2415 | 0.4215 | 0.2305 | −0.3215 |
| ABCC3 | −0.272 | −0.824 | 0.073 | −0.31 | 0.463 | 0.41 | 0.306 | −1.781 | 0.339 | 0.151 | −0.467 | 0.431 | −0.951 | 0.313 | 0.835 |
| ACAS2L | 0.5955 | 0.1105 | −0.4035 | 1.2695 | 0.9535 | −0.1845 | 0.5125 | 0.7135 | −0.1395 | −0.4175 | −0.3735 | 1.1545 | 0.3955 | −0.3115 | −0.4735 |
| ACAT1 | −2.204 | −0.519 | −2.856 | −0.378 | −1.478 | −1.532 | −2.135 | −0.628 | 0.251 | 0.300 | 0.157 | 0.7 | −0.123 | −0.826 | 0.077 |
| ACAT2 | 0.1375 | −0.3945 | −0.6265 | −1.0525 | 0.2165 | −0.5875 | −0.9495 | −0.6325 | −0.8785 | −0.6995 | −0.3155 | 0.1335 | −1.4245 | 0.2055 | 0.0795 |
| ACOX2 | −0.5945 | −0.0095 | 0.5225 | 0.0405 | −0.5335 | −0.6515 | −0.4195 | −1.9295 | −0.8275 | −0.2545 | −1.4055 | −1.0445 | −0.2365 | −0.1015 | 0.2295 |
| ACDX3 | 2.9485 | 1.2735 | −0.9205 | −0.2355 | 3.8365 | 5.1815 | 2.4595 | −0.3835 | 2.7155 | 0.8975 | −0.7415 | 3.3165 | −0.2185 | −0.0405 | 0.3175 |
| ACSL3 | −1.195 | 0.067 | 0.31 | 0.188 | 0.813 | 0.627 | −0.022 | 0.037 | 0.8 | 0.899 | 1.285 | −0.422 | −1.532 | −0.015 | 0.158 |
| ACTA1 | −0.1965 | 1.5865 | 0.5465 | 0.0485 | −0.2195 | 0.0085 | −0.0385 | 0.2035 | 0.1485 | 0.3345 | 0.2785 | 0.3025 | 0.7045 | −0.1025 | 0.1045 |
| ACTB | 0.5235 | 1.9045 | 4.3735 | 2.1515 | 3.2255 | 0.8185 | 1.2805 | 3.3295 | −0.9685 | −0.6725 | 1.4685 | −1.2775 | −1.2035 | −1.5645 | −1.3635 |
| ADAM33 | 0.752 | 0.387 | 3.308 | −0.632 | 0.283 | 2.04 | −0.538 | 0.838 | −0.075 | −1.076 | 1.301 | 1.703 | 0.018 | 0.182 | −0.109 |
| ADAMDE [symbol] | −1.0205 | −0.8615 | 0.0775 | −0.0055 | −0.6655 | −0.0835 | −0.6835 | 0.8255 | −0.7335 | −1.4065 | −1.4995 | 0.2365 | −0.6875 | −0.4385 | 0.2065 |
| ADPN | −1.6095 | −1.2775 | −1.8215 | −0.6325 | −0.9235 | −0.1645 | −1.2585 | 0.5155 | 1.6215 | 1.1025 | 0.3845 | 0.0495 | −0.1115 | −0.2835 | 1.1505 |
| ADPRHL1 | −3.9625 | −4.3655 | −5.2995 | −3.5905 | −4.1685 | −4.8395 | −3.9575 | −3.9135 | −1.0535 | −2.9375 | 0.9995 | −1.3695 | −1.8925 | −0.6045 | −2.8255 |
| ADRA2C | −1.3425 | 0.0185 | −1.4765 | −0.1685 | −0.9495 | 0.5765 | −1.3755 | −0.5975 | 1.4725 | 0.2825 | 0.4615 | 0.4535 | 0.1065 | −0.1025 | 0.6335 |
| AGR2 | −0.0105 | −0.1265 | −0.5935 | −0.3115 | 0.0275 | −0.1275 | −0.4225 | −0.2835 | 0.5435 | 0.3216 | 0.4015 | 0.7945 | 0.4285 | −0.3225 | 0.3775 |
| AGT | −0.356 | 0.308 | 0.138 | 0.13 | −0.365 | −0.321 | −1.214 | −0.13 | 0.187 | −0.291 | −0.008 | −0.231 | −0.032 | −0.813 | −0.169 |
| AGTR1 | −1.628 | −1.152 | −2.445 | −0.826 | 1.8 | −0.414 | 0.967 | −0.541 | 0.729 | 1.239 | 0.328 | 0.32 | 0.117 | −0.274 | 0.938 |
| AHCYL1 | −0.108 | 0.251 | 0.621 | 0.448 | −0.008 | −0.477 | −0.147 | 0.699 | 0.882 | 1.021 | 0.426 | −1.297 | −0.093 | −0.427 | −0.381 |
| AHNAK | −1.598 | −0.069 | 0.386 | 0.25 | 0.144 | 1.377 | 2.168 | −0.228 | −0.554 | 0.01 | 0.262 | −0.808 | −0.011 | −0.358 | 0.067 |
| AHR | 0.3465 | −0.0745 | 0.7515 | 0.0435 | −0.0245 | 0.3025 | 0.8085 | 0.0215 | −0.2835 | −0.3515 | −0.3065 | 0.3135 | −0.5745 | 0.0225 | −0.3965 |
| AIM1 | −0.7285 | 1.1855 | 1.3595 | 0.7955 | 0.6035 | 0.2685 | 0.1565 | −0.1595 | −0.2565 | −0.1965 | −0.7665 | −0.1755 | −1.2335 | −0.1225 | −0.1805 |
| AK2 | −1.8075 | −1.6645 | −2.0895 | 0.3225 | 0.1705 | −1.8525 | 0.4095 | −0.9205 | 2.6455 | 2.5305 | 3.4245 | 2.4855 | 2.8245 | 1.6935 | 1.9516 |
| AK3L1 | 0.472 | 0.014 | 0.364 | 0.009 | 0.245 | 0.794 | 0.444 | −0.829 | −0.477 | −0.188 | 0.241 | 0.348 | 0.02 | 0.666 | 0.53 |
| AK5 | −0.993 | 0.59 | 0.234 | −0.12 | −0.217 | −0.435 | −1.031 | 0.316 | −0.585 | −0.647 | 0.237 | 0.243 | −0.85 | −0.047 | −0.374 |
| AKAP1 | −0.4035 | −0.6295 | 0.0655 | −0.8765 | −0.2755 | −0.9115 | −1.1555 | −0.4675 | −0.6045 | −0.5825 | 0.0955 | 0.3185 | −1.1835 | −0.0535 | −0.3045 |
| AKR1A1 | 0.518 | 0.488 | 0.038 | 0.34 | 0.273 | −0.157 | −0.628 | −0.521 | −0.434 | −0.187 | −0.444 | 0.442 | −0.545 | −0.065 | −0.094 |
| AKR7A3 | −0.0435 | −0.2135 | −0.4225 | −0.5205 | −0.0795 | 0.6245 | 0.4045 | −0.4695 | −0.4735 | −0.9015 | −0.4135 | 0.2915 | −0.1745 | −0.9835 | −0.4285 |
| ALG5 | −0.5255 | −0.1845 | −0.9245 | −0.8815 | −0.0005 | −0.2115 | −2.0775 | −1.2855 | 0.2515 | 0.4315 | 0.2515 | 0.7225 | −0.7555 | −1.1825 | 0.0005 |
| ALG8 | 0.0585 | 0.0745 | 0.7195 | 0.3455 | −0.2495 | −0.1775 | −0.4935 | 0.2925 | 0.1775 | 0.2885 | 0.3795 | 0.3265 | −0.2715 | 0.0165 | −0.1716 |
| AMFR | −0.391 | 0.129 | −0.455 | −0.148 | 0.098 | 1.038 | 0.845 | 0.294 | −0.642 | −0.97 | −0.304 | −0.116 | −1.168 | −0.898 | −0.846 |
| AMPD2 | 0.194 | −0.154 | 2.894 | 0.483 | 0.103 | 2.044 | −0.096 | 0.823 | −1.975 | −1.343 | −1.039 | −1.779 | −1.491 | −0.831 | −1.581 |
| ANAPC11 | 0.853 | 0.406 | 0.83 | 0.265 | 0.438 | 0.79 | 2.728 | 0.155 | −0.58 | −0.607 | −0.889 | −0.032 | −0.817 | −0.604 | −0.188 |
| ANKRD22 | 0.593 | 0.595 | 1.554 | 2.935 | 0.48 | 1.668 | 2.53 | 0.449 | −1.324 | −1.246 | −0.412 | −1.391 | −1.543 | −0.659 | −0.708 |
| ANKRD27 | −0.36 | 0.23 | 0.541 | 0.697 | −0.315 | −0.475 | −0.306 | −0.49 | −0.18 | 0.093 | 0.094 | −0.171 | −0.148 | 0.175 | 0.141 |
| ANLN | 0.294 | 0.839 | 0.968 | 1.227 | 1.191 | 1.237 | 0.933 | −3.202 | 0.991 | 1.339 | 0.674 | 0.141 | 0.014 | 0.789 | 1.19 |
| ANP32A | −1.322 | 0.001 | −1.239 | 0.898 | −0.533 | −0.805 | −0.451 | −1.329 | −0.214 | −0.612 | −0.67 | −0.785 | −0.39 | −0.306 | 0.012 |
| ANXA3 | 4.9385 | 2.9005 | 3.0975 | 1.0545 | 3.2525 | 1.5915 | 1.9755 | 2.4475 | −0.8605 | −0.9865 | 0.7295 | 1.2715 | −1.5615 | −1.4815 | −1.8545 |
| APBB2 | 1.7655 | 1.4425 | −0.6816 | 2.7555 | 3.0585 | 3.2885 | 1.6625 | 1.8195 | 0.3135 | 1.0515 | −0.3725 | 2.6255 | 0.5835 | 1.6565 | 3.1095 |
| APOBEC3 | −5.19 | −4.538 | −4.815 | −2.543 | −4.003 | −4.243 | −5.067 | 4.089 | −1.002 | −1.392 | −1.385 | −1.766 | −1.427 | 0.865 | −1.899 |
| AQP5 | −2.815 | −1.733 | −3.26 | 0.333 | −2.986 | −1.522 | −1.355 | −1.906 | 2.521 | −0.15 | 2.186 | 2.014 | 1.956 | 3.784 | −0.417 |
| AR | 0.3975 | −0.2905 | 0.5495 | −0.0725 | −0.0025 | 0.6025 | 0.2585 | −0.4705 | −0.7525 | −0.9285 | −0.8855 | −0.8745 | −1.5555 | −1.0695 | −0.9355 |
| AREG | −1.24 | 0.308 | 0.811 | 0.253 | 0.736 | −0.61 | −0.059 | −0.448 | −0.415 | −0.292 | −0.065 | −0.601 | 0.113 | −0.44 | 0.118 |
| ARF1 | 0.8245 | 0.7855 | 0.1805 | −0.1755 | −0.0645 | 0.5875 | 1.1895 | 0.3605 | −0.7245 | −0.6005 | −0.5425 | −0.5375 | −1.9845 | −0.9425 | −0.8135 |
| ARHGDI [symbol] | 1.297 | 0.419 | 0.928 | 0.637 | 0.527 | 0.559 | 0.828 | 1.251 | 0.184 | 0.839 | 0.138 | −0.213 | 0.065 | 0.994 | 0.871 |
| ARL6IP | 0.5405 | −0.8145 | −0.8015 | 0.3085 | −0.7005 | −0.0665 | −0.3405 | −1.1915 | −0.1735 | −0.1645 | −0.2585 | −0.0005 | −1.3495 | 0.2965 | −0.2405 |
| ARL6IP2 | 1.702 | −0.366 | 2.153 | −1.058 | −0.163 | −1.185 | −0.509 | 0.939 | −2.188 | −0.909 | 1.171 | −0.272 | −0.881 | −0.443 | −1.695 |
| ARMCX2 | 1.736 | −0.44 | 2.053 | −1.108 | −0.413 | −1.184 | −0.493 | 0.876 | −1.884 | −1.024 | 1.182 | −0.223 | −0.494 | −0.422 | −1.987 |
| ARNT2 | 1.214 | −1.141 | 1.316 | 1.304 | 0.312 | 1.996 | 2.143 | 1.967 | 0.164 | 0.303 | −0.178 | 1.338 | −1.011 | 0.826 | 1.105 |
| ARNT2 | −0.0925 | 1.3595 | 0.6195 | 0.0575 | 0.6035 | 0.1775 | 0.2165 | 0.8435 | 0.2635 | 0.0815 | 0.8985 | −0.8855 | −0.9375 | −0.4045 | −0.1815 |
| ARP3BET [symbol] | 0.4995 | 0.5605 | −0.0825 | 0.1465 | 0.1395 | 0.4205 | 1.4705 | −0.3155 | −0.0465 | −0.4505 | −0.2365 | 0.0275 | −0.6785 | −0.8455 | −0.8095 |
| ARPC1B | 0.708 | −0.17 | 2.197 | 0.928 | −0.016 | −0.698 | −0.16 | 1.597 | 0.6 | 0.459 | 0.159 | 0.75 | −0.019 | 0.155 | 0.106 |
| ARPC5L | 0.7815 | 1.3195 | 1.3025 | 0.7945 | 1.4555 | 1.0085 | 1.3605 | 0.4945 | −0.5925 | −0.6645 | −0.9605 | −0.6005 | −1.5915 | −0.5805 | −0.6915 |
| AS89 | 0.991 | −0.14 | 0.733 | 1.417 | 0.189 | 0.4 | 0.251 | 0.477 | 0.234 | 0.224 | 0.009 | 0.054 | −0.543 | 0.133 | −0.102 |
| ASF1A | 1.3525 | 2.5925 | 1.9675 | 1.1105 | 0.4525 | −0.0085 | 1.0625 | 0.0355 | −1.4065 | −1.1735 | −0.9025 | −1.4775 | −1.5475 | −0.5415 | −1.0075 |
| ASH2L | 1.987 | 1.152 | 1.393 | 0.727 | 0.738 | 1.854 | 3.391 | 1.444 | −1.39 | −2.013 | 0.2 | −1.574 | −1.073 | −0.984 | −1.483 |
| ASK | −0.113 | 0.71 | −0.44 | 0.354 | 0.072 | 1.262 | 1.545 | 0.536 | 1.123 | 0.455 | 0.427 | 0.585 | 0.685 | 0.806 | 1.119 |
| ASPM | 1.1445 | 1.5545 | 2.2075 | 0.9625 | 1.2345 | 1.1635 | 3.4045 | 0.1815 | −1.3725 | −1.6495 | −0.8295 | −1.5955 | −1.7155 | −0.9305 | −1.4555 |
| ASS | 0.3875 | −0.4005 | 0.8465 | −0.2145 | 1.1995 | 0.2175 | 0.6085 | 1.9925 | −0.1235 | −0.3765 | 0.3755 | −0.1255 | −0.0165 | −0.2885 | −0.3965 |
| ATAD2 | 0.0565 | 0.2625 | 0.0005 | 0.7345 | −0.1915 | 1.0945 | 0.5205 | 0.7685 | −1.0135 | −0.6795 | 0.0716 | −0.9975 | 0.1035 | −0.1985 | −0.3105 |
| ATF5 | 0.53 | 0.252 | 0.042 | −0.208 | 0.502 | −0.434 | 0.029 | −0.218 | 0.059 | 0.598 | −0.098 | −1.096 | −1.058 | 0.236 | −0.393 |
| ATF7IP2 | −0.23 | 0.181 | 0.699 | 0.367 | 0.262 | 0.102 | 0.871 | −0.271 | −0.313 | −0.334 | −0.044 | 0.64 | −0.878 | 0.278 | 0.041 |
| ATP2B1 | −0.099 | 0.011 | −0.348 | 0.084 | 0.358 | 0.561 | 0.174 | 0.132 | −0.076 | −0.17 | −0.197 | 0.422 | −0.432 | 0.222 | −0.006 |
| ATP5G1 | −0.4105 | −0.5425 | −0.5125 | −0.6945 | −0.0235 | −0.3955 | −0.5095 | −0.4855 | −0.6865 | −0.9315 | −0.0735 | −0.6035 | −0.1445 | −0.5485 | −0.7945 |
| ATP5O | 3.035 | −0.912 | −1.252 | 0.213 | 0.28 | 1.208 | 2.301 | 3.589 | −0.36 | −0.601 | −1.035 | 1.221 | −0.211 | 0.818 | 1.853 |
| ATP6AP1 | −1.197 | −0.103 | −0.053 | −0.718 | 1.045 | −0.258 | 0.781 | −0.456 | −0.407 | 0.613 | 0.484 | 0.385 | 0.204 | 0.015 | 0.272 |
| ATP6V0A4 | 0.241 | −0.088 | −0.628 | −0.482 | 0.109 | −0.233 | −0.054 | −1.135 | −0.938 | −0.144 | −0.099 | −0.122 | −1.353 | −0.586 | −1.384 |
| ATP6V1B [symbol] | 0.1875 | −0.5465 | 0.1665 | 0.0585 | −0.4945 | −0.6795 | 0.2925 | −0.7435 | −0.6095 | −0.6125 | 0.1885 | −0.2495 | −0.3675 | −0.5175 | −0.8155 |
| ATP6V1G [symbol] | 0.088 | 0.716 | 0.101 | 0.224 | 0.273 | −0.891 | −0.38 | −0.954 | −0.127 | 0.234 | 0.058 | 0.312 | −0.112 | −0.013 | −0.197 |
| ATPIF1 | −0.1015 | 1.4575 | 1.2005 | 1.9325 | 1.6695 | 2.3855 | 3.0975 | 1.3555 | 1.7465 | 0.3295 | 0.1205 | −1.3465 | −0.4425 | 8.9135 | 0.9095 |
| ATXN10 | 0.338 | 0.139 | 0.385 | 0.454 | 0.005 | −0.376 | −0.426 | −0.14 | 0.211 | 0.25 | 0.478 | 0.128 | 1.001 | 0.741 | 0.002 |
| B3GNT5 | −0.8245 | −0.8155 | −0.8105 | −0.3855 | 0.0775 | −0.3285 | −0.8435 | −0.9085 | 0.3285 | 0.0875 | 0.2405 | −0.0005 | 0.2925 | 0.2875 | 0.0495 |
| BACE2 | 0.7935 | −0.0865 | 0.6695 | 0.8766 | 0.0175 | 0.3395 | 0.9395 | −1.1395 | −0.3905 | −0.1745 | −0.0965 | −0.3335 | −0.0005 | −0.2195 | −0.3085 |
| BAG1 | −1.4835 | −0.9315 | −0.6835 | −0.7055 | −1.3455 | −0.9215 | 0.0275 | −0.1665 | 0.9295 | −0.2465 | 0.0275 | −0.0275 | 1.0575 | 0.0685 | −0.2015 |
| BAG4 | 1.4985 | −0.4725 | 0.0555 | −0.3645 | −0.9885 | −0.6175 | 0.4485 | 0.0975 | −1.0685 | −1.7855 | −0.0745 | 0.3925 | −0.2945 | 0.5235 | −0.0925 |
| BAI2 | −1.031 | −0.982 | −1.036 | −0.631 | −1.217 | −1.771 | −1.071 | −0.325 | −0.933 | −0.736 | 0.008 | −0.783 | −0.848 | −0.512 | 0.262 |
| BAMBI | 2.889 | 0.51 | 0.381 | 0.396 | 1.691 | 1.429 | 0.068 | 4.085 | −0.172 | −0.148 | −0.477 | 0.309 | 0.439 | 0.127 | 0.892 |
| BCAS1 | −4.009 | −4.51 | −5.594 | −3.733 | −4.626 | −3.53 | −4.538 | −3.586 | −1.657 | −3.301 | −0.845 | −2.762 | −1.461 | −2.269 | −4.593 |
| BCL2L14 | −0.1045 | −1.0055 | −1.6395 | 0.2735 | −0.8375 | 0.8075 | −0.4215 | 3.9355 | 0.7745 | 1.1175 | 0.6735 | 1.2615 | 1.0215 | 0.7285 | 0.9345 |
| BCMP11 | 1.061 | −0.68 | −1.12 | 0.873 | 0.09 | 1.017 | 0.304 | 1.261 | 0.007 | 0.692 | 0.33 | 1.035 | 0.896 | 1.905 | 0.483 |
| BEX1 | −3.0895 | −0.7165 | −0.9525 | −0.5665 | 0.3645 | 1.1715 | −2.7005 | −0.3955 | −0.1935 | −0.4355 | 0.3545 | −1.4255 | −1.4875 | −1.5015 | −1.9605 |
| BEX2 | 0.0395 | −0.1355 | −1.3325 | −0.2615 | −1.3185 | −0.0165 | −0.2485 | 0.2205 | −0.3425 | −0.4635 | −0.2355 | 0.3465 | 0.5315 | −0.0745 | 0.2315 |
| BF | 1.361 | −0.44 | −0.857 | −0.61 | −0.292 | −0.121 | −1.054 | −0.255 | −0.587 | −0.659 | −0.731 | −0.052 | −0.545 | −0.529 | −0.689 |
| BFSP1 | 1.004 | −0.171 | 2.187 | 0.564 | 1.192 | 1.165 | 1.804 | −0.509 | 0.181 | 0.28 | 0.072 | −1.471 | 0.084 | 0.12 | −0.144 |
| BIK | 0.85 | 1.514 | 2.197 | 0.894 | −0.209 | 1.059 | 2.166 | 1.265 | −1.534 | −1.684 | 0.658 | −0.652 | −1.412 | −0.527 | −1.858 |
| BIRC2 | 0.5295 | 0.6795 | 1.3855 | 0.4555 | 0.4085 | 0.5105 | 0.3005 | −0.3155 | 0.0785 | −0.2885 | −0.0325 | 0.3025 | −0.3975 | 0.0785 | −0.4125 |
| BIRC5 | −0.7425 | 0.6105 | 0.5335 | 0.2765 | −0.7325 | 1.2805 | 0.2185 | 0.0375 | −0.1995 | 0.6235 | 0.0645 | −0.4955 | −1.5205 | 0.1065 | 0.6525 |
| Bit1 | −1.2455 | −0.8085 | 0.8315 | −0.4055 | −0.2455 | −0.8575 | −0.9705 | −0.8805 | −0.0445 | −0.3975 | 0.5165 | −0.0585 | −0.4395 | −0.3255 | −0.1685 |
| BIVM | 0.285 | 0.214 | 2.388 | 0.911 | 0.557 | 0.419 | 2.16 | 1.038 | −0.08 | −0.364 | 0.096 | 0.774 | 0.163 | 0.124 | −0.429 |
| BLVRA | 4.397 | 4.042 | −0.604 | −0.034 | 2.559 | 3.413 | 1.655 | 3.202 | 0.872 | 0.56 | 0.605 | 1.306 | 0.98 | 0.538 | −0.312 |
| BM039 | −0.772 | −0.128 | −0.196 | 0.488 | 0.001 | −0.13 | 0.538 | −0.424 | 0.874 | 0.635 | 1.134 | 0.777 | 0.623 | 0.816 | 1.25 |
| BMP7 | 0.828 | 0.738 | −1.383 | 0.662 | −0.494 | −1.992 | −1.194 | 0.341 | −0.2 | −0.478 | 0.436 | −0.224 | −0.764 | 0.014 | −0.544 |
| BOC | 1.3635 | 0.2055 | 0.2485 | 1.2225 | 0.3525 | 0.7085 | 0.0095 | 0.1045 | −0.9955 | 0.1225 | −0.2395 | 0.3695 | −0.5685 | −0.7345 | −0.4105 |
| BRCA1 | 0.4195 | 0.0685 | 0.5995 | −0.0285 | 1.0145 | 0.7785 | 0.8405 | −0.3905 | 0.2625 | −0.1825 | 0.9925 | −0.0145 | 0.0855 | −0.6095 | 0.2135 |
| BRF2 | 0.105 | 0.02 | −0.105 | 0.05 | 0.283 | 0.057 | 0.011 | 0.334 | −1.187 | −1.325 | −0.311 | −0.795 | −0.389 | −0.593 | −0.925 |
| BRI3 | −1.493 | −0.928 | −0.633 | 0.029 | −1.069 | −1.34 | −0.491 | −1.056 | −1.132 | −2.029 | −0.197 | −0.242 | −1.783 | 0.097 | −1.105 |
| BRI3BP | 0.271 | −0.585 | −0.104 | −0.108 | −0.378 | −1.653 | −0.812 | −0.672 | 0.023 | 0.217 | 0.304 | 0.562 | −0.336 | 0.382 | 0.094 |
| BSPRY | 1.3585 | 2.6995 | 0.8175 | 1.7215 | 1.3376 | 0.6185 | 2.5746 | 0.7295 | 0.8725 | 0.8605 | 0.0755 | 0.8405 | −0.5165 | 0.0725 | −0.1235 |
| BTF3 | 1.0345 | 0.7945 | 1.5515 | 1.7305 | 1.3555 | 0.7935 | −0.4185 | 1.6035 | −0.7845 | 0.0985 | −0.1245 | −0.1605 | −0.6065 | −0.1765 | −0.1695 |
| BTG3 | 1.58 | 1.671 | 1.386 | 0.97 | 1.316 | 1.588 | 3.067 | 0.913 | −0.742 | −1.357 | −0.377 | −0.962 | −1.272 | −0.661 | −1.231 |
| BγN2A2 | 0.655 | −0.185 | 0.398 | 1.038 | −0.352 | −0.421 | 0.261 | −0.288 | −0.913 | −0.958 | −0.371 | −0.004 | −1.114 | −0.346 | −0.888 |
| BUB1 | 1.4405 | −0.3775 | 0.4385 | 0.5215 | 0.5965 | 0.1185 | 1.2315 | −0.3355 | 0.1725 | 0.1045 | −0.3975 | −0.9155 | 0.9215 | 0.5045 | −0.4155 |
| BUB3 | 1.52 | 0.528 | −0.009 | 0.681 | 0.657 | 0.205 | −0.736 | −0.285 | −0.119 | −0.667 | 0.393 | 0.361 | −0.254 | 0.046 | 0.206 |
| BXDC1 | 0.81 | 0.087 | −0.42 | −0.161 | 0.58 | −0.22 | 0.746 | 0.377 | 0.011 | −0.774 | −0.932 | −0.239 | −1.012 | −0.827 | −0.946 |
| BZRP | −1.168 | −3.737 | −4.816 | −1.271 | −0.901 | −0.284 | −2.204 | −1.735 | 1.952 | 1.49 | 1.196 | 0.882 | 0.121 | −0.516 | 1.684 |
| BZW1 | 1.5075 | 0.6215 | 0.8795 | 0.2145 | 1.7015 | 0.8075 | 0.5195 | 0.9725 | 0.8555 | 1.0095 | 0.4295 | 0.5335 | −0.0455 | 0.5565 | 0.7085 |
| C10orf116 | 0.7965 | −0.6585 | −0.4455 | −0.0395 | 0.3415 | −0.4855 | −0.9755 | −0.3305 | 0.2525 | 0.4815 | 0.1485 | −2.4045 | −0.1005 | 0.0585 | −0.2075 |
| C10orf38 | −0.01 | 0.355 | 0.693 | −0.374 | 0.989 | −0.42 | −0.355 | 0.004 | 0.479 | 0.915 | −0.111 | −1.545 | −1.708 | −1.201 | −0.243 |
| C10orf4 | 1.4885 | 0.8195 | 0.4315 | −0.0025 | 1.6285 | 0.8485 | 0.3815 | −0.1915 | 0.0635 | −0.1835 | 0.2285 | −0.4345 | −0.2605 | −0.4065 | −0.8605 |
| C10orf45 | −0.0395 | −0.7075 | −0.8345 | −0.2455 | −0.3825 | −0.8535 | −0.5955 | 0.1405 | −0.0005 | −0.2165 | 0.2735 | 0.5645 | 0.4245 | −0.1575 | 0.0965 |
| C10orf7 | 0.2145 | −0.3115 | 0.2205 | −0.2395 | 0.2325 | 0.0835 | 0.0995 | −0.0415 | −0.1935 | −0.4685 | 0.0485 | 0.4245 | −0.7255 | −0.8425 | −0.5855 |
| C10orf82 | 1.863 | 0.815 | 0.028 | −0.262 | 1.188 | 1.018 | −0.94 | 2.324 | 0.42 | 0.928 | 0.978 | −0.184 | 0.978 | 1.306 | 0.067 |
| C11orf24 | −1.4715 | −0.6945 | −1.0495 | −0.3395 | −0.7035 | 0.1095 | −0.4015 | −0.0865 | 0.2135 | 0.7035 | 0.6785 | 1.0955 | 0.5045 | 0.3395 | −0.0005 |
| C12orf14 | 0.206 | 1.171 | 0.154 | 0.385 | 0.48 | 1.408 | 0.902 | 0.954 | −1.454 | −1.072 | −0.896 | −0.325 | −0.308 | −0.741 | −1.153 |
| C13orf21 | −1.379 | −0.805 | −0.048 | 0.085 | −0.475 | −0.508 | 0.063 | −0.434 | 0.231 | 0.123 | 0.748 | 0.878 | 0.599 | 0.637 | 0.401 |
| C13orf23 | 0.627 | 1.21 | 1.099 | 0.504 | 0.839 | 1.02 | 1.6 | 0.493 | −0.458 | −0.937 | 0.157 | 0.345 | −1.255 | −0.981 | −1.224 |
| C14orf94 | −0.661 | −0.102 | −2 | −0.77 | −0.811 | −1.72 | −1.181 | 0.76 | 0.86 | 0.5 | 0.7 | 0.382 | 0.031 | −0.1 | 0.435 |
| C15orf23 | −0.3265 | 1.9285 | −0.4825 | −0.4125 | −0.3775 | −0.2565 | −0.5275 | −0.7435 | 1.0785 | −0.4525 | 0.5415 | −0.5625 | 0.6215 | −0.1545 | −0.5995 |
| C16orf45 | 0.248 | −0.369 | 0.304 | −0.085 | −0.035 | −0.424 | −0.518 | −1.335 | −1.251 | −0.23 | −0.835 | −0.638 | −0.348 | −1.012 | −0.413 |
| C17orf37 | −0.704 | 0.815 | 0.091 | 0.002 | 0.042 | 0.134 | −0.665 | −0.236 | 0.064 | −0.182 | 0.755 | −0.129 | −0.605 | −0.218 | −0.394 |
| C18orf1 | −2.144 | −1.766 | −1.672 | −1.045 | −1.722 | −0.998 | −1.297 | −0.928 | −0.342 | −1.028 | 0.112 | −0.233 | 0.576 | 0.067 | −0.428 |
| C19orf10 | −1.0305 | 0.1425 | −0.1215 | −0.0905 | −0.9576 | −1.0275 | −0.4615 | −0.5175 | −0.1485 | −0.3045 | 0.5515 | 0.0235 | −0.8485 | 0.1305 | −0.0645 |
| C19orf32 | 0.0965 | −1.1435 | −0.4555 | −0.5295 | 0.0795 | −0.8255 | 0.2525 | 0.1235 | 1.1985 | 0.9805 | 0.0485 | −0.6745 | −0.3465 | 0.0835 | 0.4075 |
| C19orf33 | −3.01 | −1.96 | −1.893 | −1.948 | −0.484 | −0.838 | −2.445 | −0.607 | −0.773 | −2.446 | −0.355 | −1.043 | −1.688 | −0.145 | −3.033 |
| C1orf24 | −0.098 | 0.168 | −0.97 | −1.223 | 0.778 | −1.217 | −1.03 | −0.017 | −1.474 | −1.863 | −1.307 | −0.985 | −1.548 | −1.216 | −1.426 |
| C1orf34 | −1.0075 | −0.5055 | −1.8855 | −0.8655 | −0.7365 | 0.9895 | −0.3385 | −0.2955 | −0.3475 | −1.0845 | 0.0465 | −0.8075 | 0.2245 | −0.3265 | −0.4555 |
| C20orf103 | −0.0545 | −1.0435 | −0.4855 | −0.7995 | −0.5315 | −0.8025 | 0.0005 | −1.8575 | −0.4555 | −0.5715 | −1.0105 | −1.1585 | −1.6585 | −0.6335 | −0.4095 |
| C20orf149 | 0.162 | −0.382 | 0.043 | −0.533 | −0.502 | −0.639 | −0.7 | −0.53 | 0.263 | −0.236 | −0.252 | −0.397 | −0.223 | −0.489 | −0.230 |
| C20orf23 | 0.94 | −0.119 | −0.68 | −0.398 | −0.586 | −0.593 | −1.148 | −0.812 | 0.165 | 0.126 | 0.453 | −0.141 | 0.316 | 0.088 | 0.004 |
| C20orf3 | 0.187 | −0.108 | −0.543 | −0.169 | 0.56 | 1.394 | −0.126 | 0.018 | −0.037 | 0.29 | 0.178 | 0.253 | −0.409 | 0.322 | 0.261 |
| C20orf35 | 1.663 | −0.013 | −0.723 | 0.041 | 0.522 | −0.098 | 0.003 | 0.27 | −0.385 | −0.287 | −0.199 | −1.487 | −0.36 | −0.402 | −0.085 |
| C20orf43 | −0.1425 | 0.1405 | −0.2935 | −0.1105 | −0.3125 | −0.1465 | 0.7635 | −0.1075 | −0.3425 | 0.3015 | 0.1585 | −0.0095 | −0.2045 | 0.3925 | 0.1975 |
| C20orf8 | −0.036 | 1.066 | 1.08 | 0.424 | 0.165 | 0.524 | 0.712 | 0.268 | −0.385 | −0.814 | 0.555 | 0.591 | −0.487 | −0.109 | −0.881 |
| C21orf45 | 1.998 | 0.425 | −0.44 | −0.024 | 1.482 | 1.941 | 0.238 | 0.306 | 0.081 | 0.237 | 0.096 | 0.25 | −0.147 | 0.158 | −0.068 |
| C22orf18 | 0.35 | −0.504 | 0.131 | −0.054 | 0.282 | −0.12 | −0.839 | −1.019 | 0.278 | 0.53 | 0.216 | 0.002 | 0.293 | 0.173 | 0.276 |
| C2F | −1.708 | −1.156 | −1.782 | −0.753 | −1.173 | −0.896 | −1.69 | −0.52 | 1.154 | −0.015 | 1.167 | −0.285 | 1.156 | −0.204 | −0.547 |
| C2orf17 | 0.18 | 0.884 | 0.652 | −0.078 | 0.673 | 0.029 | 0.604 | −0.079 | −0.928 | −0.851 | −0.455 | 0.096 | −1.09 | −0.395 | −1.397 |
| C4A | 1.021 | 1.069 | 0.378 | 0.076 | 1.043 | −0.05 | −0.32 | 0.198 | 0.11 | −0.21 | 0.352 | −0.009 | −0.033 | −0.028 | −0.084 |
| C5orf13 | 0.542 | 0.177 | 0.012 | 0.447 | 0.656 | 0.743 | 0.201 | 0.501 | 0.687 | 0.705 | 0.385 | 0.215 | −1.037 | −0.019 | −0.198 |
| C6orf108 | 0.927 | 1.567 | 0.029 | 1.124 | 0.544 | −0.134 | 1.953 | 0.248 | −0.002 | 0.255 | 0.003 | 0.5 | 0.155 | 0.442 | 0.422 |
| C6orf166 | −0.573 | −0.364 | −0.377 | −0.524 | −0.795 | −1.664 | −1.481 | −1.232 | −1.204 | −1.138 | −0.583 | −1.651 | −0.689 | −0.197 | −0.964 |
| C6orf192 | 0.2915 | 1.3605 | −0.2835 | −0.0425 | 1.1205 | 0.4245 | −0.4355 | −0.2545 | −0.9785 | −1.1495 | −0.7835 | 0.3455 | −1.6455 | −0.3155 | −0.2335 |
| C6orf211 | −3.098 | −1.492 | −1.681 | 0.03 | −0.555 | −0.879 | −1.028 | −1.558 | −0.095 | −0.221 | 0.038 | −0.368 | −0.201 | 0.048 | 0.13 |
| C6orf49 | −0.3555 | −0.9775 | 0.4235 | −0.1055 | −0.6135 | −0.5815 | −0.0795 | −0.0075 | −1.1625 | −1.2195 | −0.7525 | −0.5755 | −1.1855 | −1.0445 | −1.4905 |
| C6orf85 | −0.5245 | −0.5695 | −0.4825 | −0.9245 | −0.4355 | 0.4585 | −0.3745 | −0.0745 | 0.1205 | −0.3275 | 0.4535 | −0.9185 | 0.1125 | −0.0995 | −0.5105 |
| C7orf24 | −2.037 | −0.679 | 0.882 | −0.846 | −2.286 | −0.575 | −0.647 | −1.046 | 0.389 | 0.06 | 1.18 | −0.628 | 0.485 | 0.412 | −0.852 |
| C9orf10 | −0.646 | 1.027 | −1.632 | 0.635 | −0.344 | 2.032 | 1.193 | 1.063 | 1.417 | 0.928 | −0.02 | 1.611 | 1.681 | 1.706 | 2.178 |
| C9orf121 | −0.847 | −0.324 | −0.801 | −0.353 | −1.128 | −0.08 | 0.338 | 0.384 | 0.101 | −0.179 | 0.705 | −0.44 | −0.851 | 0.217 | 0.015 |
| C9orf58 | 0.0055 | −0.3675 | −0.0115 | −0.2425 | −0.2055 | 0.0055 | 0.6085 | 0.4015 | −0.2975 | −0.6505 | −0.5945 | 0.3055 | −0.6705 | −0.1345 | 0.0665 |
| C9orf88 | −2.319 | −2.195 | −2.364 | −2.072 | −1.11 | −2.201 | −2.078 | −1.389 | −1.489 | −2.061 | −0.038 | −2.158 | −1.302 | −0.452 | −0.431 |
| C9orf9 | −1.142 | 0.636 | 0.341 | 0.032 | 1.765 | 0.514 | 0.627 | 0.169 | −0.399 | −0.755 | −0.223 | 0.63 | 0.131 | 0.036 | −0.526 |
| CA12 | −0.576 | 0.751 | 0.353 | −0.129 | 2.2 | 0.521 | 0.861 | 0.802 | −0.553 | −1.14 | −0.447 | 0.803 | −0.273 | −0.115 | −0.373 |
| CA8 | 0.17 | 0.554 | −0.45 | 0.12 | −0.06 | −0.229 | −0.153 | −0.07 | 0.345 | 0.068 | −0.042 | 1.018 | 0.546 | 0.643 | 0.14 |
| CA8 | 3.1095 | 0.0075 | 0.1825 | −0.1435 | 0.4165 | −0.0625 | −0.0415 | 0.1205 | 0.1855 | 0.0095 | −0.0555 | −0.1195 | 0.0115 | −0.0605 | −0.1505 |
| CACNA1G | −0.025 | 0.789 | 2.588 | 0.713 | 0.162 | 0.484 | 0.944 | 0.618 | 0.059 | 0.459 | 0.006 | 0.048 | −0.316 | −0.389 | −0.145 |
| CALCA | 3.217 | −0.4 | 0.933 | 1.482 | 2.184 | 2.192 | 0.703 | 1.345 | −0.769 | −1.082 | −0.888 | 0.547 | −0.309 | 0.897 | −0.713 |
| CALML4 | −1.6505 | −1.1935 | −2.4265 | −0.8275 | −1.4745 | −1.7215 | −1.4705 | −2.5725 | 0.5755 | 0.2125 | 1.2035 | −0.8405 | −0.8275 | 0.0065 | −0.4405 |
| CALML5 | −0.7965 | −0.8425 | −0.2315 | 0.3895 | −0.4415 | 0.8875 | −0.4825 | −1.1295 | −0.2745 | −0.7145 | −0.8975 | −0.8795 | −0.3045 | −0.3205 | −0.0735 |
| CaMKIINa [symbol] | 0.119 | −0.055 | 1.006 | 0.011 | 0.088 | −0.896 | 0.214 | 0.322 | −0.772 | −0.811 | −0.911 | −2.22 | −1.534 | −0.388 | −0.318 |
| CAP2 | −0.986 | −1.806 | −1.815 | −1.336 | −0.511 | −1.248 | −1.216 | −0.402 | −0.831 | −1.211 | 1.098 | −0.82 | 0.277 | −0.721 | −1.472 |
| CAP350 | 3.4475 | 1.8305 | 5.0525 | 2.3775 | 0.7285 | 2.7985 | 3.3735 | 1.9795 | 0.0565 | 1.6355 | 1.3135 | 3.8385 | 0.1776 | 2.0735 | 1.7855 |
| CAPN13 | −0.866 | −0.068 | −0.517 | −0.692 | −0.476 | 0.845 | −0.709 | −0.854 | −0.486 | −0.779 | −0.31 | −0.471 | −0.743 | −0.164 | −0.873 |
| CAPN9 | 0.2945 | 0.1715 | −0.3485 | −0.2875 | 0.0985 | 0.1865 | −0.3805 | 0.4165 | −0.0805 | −0.3045 | −0.6725 | −0.2045 | 0.0295 | −0.3065 | −0.5645 |
| CARD14 | 0.5225 | 0.8975 | 1.6935 | 1.1355 | 1.8145 | 0.9595 | 1.0035 | −0.0115 | 0.6755 | 0.7395 | 0.9845 | 0.8895 | −0.7595 | 0.5155 | 0.4445 |
| CART | 0.2775 | 0.3565 | 0.8485 | 0.3065 | 0.2745 | −0.2605 | −0.0545 | 0.1725 | −0.5055 | −0.8045 | −0.3385 | −1.7575 | −0.7495 | −0.5795 | −0.9295 |
| CASP1 | 0.002 | 0.108 | 0.438 | 0.672 | 0.258 | 0.444 | 0.148 | −0.606 | 0.317 | 0.211 | 0.259 | −0.643 | −0.259 | −0.572 | −0.53 |
| CASP3 | −0.369 | −4.235 | −0.36 | −0.079 | −0.299 | −0.51 | −0.291 | −0.561 | 0.419 | 0.68 | −0.43 | −0.02 | 0.346 | −0.275 | −0.227 |
| CASP7 | 0.8275 | 0.7235 | 0.4545 | 0.1845 | 0.2135 | 0.5745 | −0.4145 | 0.4145 | 0.1445 | −0.5755 | 0.5245 | 0.5505 | −0.1155 | 0.2345 | 0.2185 |
| C9LN1 | −0.593 | −0.163 | 0.001 | 0.866 | −0.283 | 0.118 | 0.038 | 1.041 | 0.134 | −0.232 | −0.087 | −0.107 | 0.373 | −0.132 | 0.497 |
| CBR1 | −0.2535 | −0.1915 | 0.5545 | −0.2625 | 0.0665 | −1.2595 | 0.5675 | −1.6825 | −0.7075 | −0.4575 | −0.4305 | 0.2365 | −0.7955 | −0.0905 | −0.3735 |
| CBR3 | −0.013 | 0.349 | −0.444 | −0.113 | 0.166 | −0.135 | −0.112 | 0.003 | 1.038 | 0.676 | 0.74 | 0.237 | 1.237 | 0.266 | 0.158 |
| CBR4 | −0.6235 | −1.2765 | −2.1095 | 1.5905 | −0.8405 | −2.1795 | −1.9555 | 0.1135 | 0.9735 | 1.3905 | 1.2335 | 3.2905 | 3.6695 | 3.1835 | 3.6805 |
| CCL24 | 0.506 | 1.002 | 1.942 | 1.198 | 0.624 | 0.673 | 1.98 | 0.028 | −0.35 | −0.846 | −0.069 | −0.102 | −0.136 | 0.086 | −1.223 |
| CCL29 | −0.592 | 1.157 | −2.323 | −1.398 | 0.356 | −0.114 | −1.87 | −0.822 | −0.631 | −0.351 | 2.186 | −0.207 | −1.419 | 0.467 | 0.009 |
| CCNB2 | 1.6175 | 0.1276 | 1.2155 | 1.0185 | −0.0145 | 0.2295 | 2.8755 | 2.3395 | 0.0795 | −0.5195 | 0.1445 | 0.4815 | 0.7815 | −0.1455 | −0.1085 |
| CCND1 | −0.697 | −0.814 | 0.209 | −0.387 | −0.249 | 0.263 | 1.362 | −1.688 | −1.123 | −0.887 | −0.421 | −0.359 | −1.181 | −0.725 | −0.899 |
| CCNE1 | 0.6685 | −0.1265 | −0.0805 | 0.0635 | 0.2425 | 1.0065 | −0.6505 | −0.7275 | 0.4605 | −0.1045 | −0.0005 | 0.0005 | −0.0445 | −0.3765 | −0.5135 |
| CCNG2 | −0.007 | 3.267 | 3.132 | 1.202 | 1.727 | 1.32 | 0.884 | 2.098 | −0.395 | −0.237 | −0.507 | 0.538 | 0.587 | −0.133 | −0.306 |
| CCNH | −0.0535 | 0.9995 | 2.5185 | 1.0315 | 3.1535 | 0.3685 | 1.3655 | 1.8135 | −0.1495 | 0.8095 | 0.4475 | 0.1895 | −0.5285 | 0.0245 | 0.1205 |
| CD38 | −0.3975 | 2.6655 | 1.4875 | 0.3255 | 0.0205 | 0.9555 | −0.1645 | 1.7165 | 1.2935 | 0.7475 | 0.9605 | 0.1815 | 0.6655 | 0.0495 | 0.3325 |
| C030 | 0.9415 | 0.6415 | 0.6875 | 0.8735 | 1.4455 | −0.4655 | −0.1065 | −0.3605 | −0.3835 | −0.3235 | −0.4885 | 0.2065 | 0.6315 | −0.2035 | 0.1385 |
| CD4 | −0.0525 | −0.4435 | −0.5415 | 0.1065 | 0.3875 | 0.6705 | 0.6285 | −0.4575 | 1.3145 | 0.9615 | 0.9385 | 0.9655 | 0.2585 | 0.5415 | 1.1785 |
| CD58 | 1.24 | 0.699 | 1.427 | 0.957 | 0.181 | 0.888 | 1.73 | 0.144 | −0.385 | −1.332 | −0.326 | −0.306 | −0.928 | −0.452 | −0.834 |
| CD50 | 0.6245 | 2.4935 | 0.6975 | 0.5985 | 0.5435 | 0.0585 | 0.7195 | 0.7395 | −0.8695 | −1.8025 | 0.0775 | 0.5505 | −0.6725 | −0.0725 | −0.2955 |
| CDC2 | 1.8035 | 0.8235 | 1.5515 | 1.7815 | 0.9305 | −0.2835 | 2.8465 | 1.1985 | −1.3575 | −1.5855 | −0.3865 | −1.2235 | −2.0745 | −1.4835 | −2.2385 |
| CDC6 | 1.2275 | 1.4335 | 1.5435 | 0.9545 | 0.5025 | 0.9205 | 2.2225 | 0.8875 | −1.8865 | −1.7885 | −0.3775 | −0.1505 | −1.9035 | −0.3505 | −1.9885 |
| CDCA1 | 2.081 | 2.884 | 2.733 | 2.026 | 0.693 | 0.558 | 3.236 | 2.787 | −0.562 | −0.541 | −1.208 | 1.182 | 0.159 | 0.978 | 0.042 |
| CDCA5 | −0.203 | −1.305 | 1.482 | −0.224 | −0.748 | −0.368 | −0.877 | −1.999 | −0.771 | −0.842 | −0.671 | −0.568 | −0.846 | 0.247 | −1.344 |
| CDCA7 | 1.6295 | 1.6375 | 2.7675 | 1.1085 | 2.1425 | 2.3445 | 1.7815 | −0.5845 | 0.5475 | 0.9245 | 1.8715 | 1.0055 | 0.2725 | 1.1385 | 0.2295 |
| CDH1 | 0.777 | 0.442 | −0.363 | 0.242 | 0.023 | 0.126 | −0.484 | 1.044 | 1.095 | 0.022 | 1.424 | −0.41 | −0.584 | −0.151 | −1.126 |
| CDH3 | 2.6975 | −0.0075 | 3.3045 | 2.5665 | −0.4555 | −1.3405 | 2.5655 | 1.8155 | 0.2515 | −0.9305 | 0.0185 | −0.7915 | 0.5025 | −0.7975 | −0.4935 |
| CDK2 | 1.0495 | 0.0905 | −0.0575 | 0.7225 | 1.3535 | 0.7025 | 2.3925 | 0.0385 | 0.3495 | −0.3465 | 0.8445 | −0.6505 | −0.3155 | −1.2515 | −1.0635 |
| CDKN2A | 0.2905 | 0.3335 | 0.7975 | 0.8765 | 0.8365 | 0.8035 | 3.2785 | −0.0665 | −0.1175 | −0.7035 | −0.4545 | −0.3755 | −1.0545 | −0.8295 | −1.0765 |
| CDKN2D | 0.525 | 0.716 | 1.38 | 0.515 | 0.384 | 0.231 | 2.148 | 2.675 | −0.32 | −0.762 | 0.486 | 1.671 | −1.575 | 1.078 | −0.218 |
| CDKN3 | 0.281 | 0.156 | 0.626 | 0.787 | −0.047 | 0.414 | 2.218 | 0.739 | 0.28 | −0.031 | −0.188 | −1.081 | −0.142 | −0.387 | −0.084 |
| CDT1 | −0.8165 | −0.9935 | −0.9845 | −1.2655 | −0.3916 | −1.3565 | −1.0145 | −1.1395 | −0.7795 | −1.4085 | 0.0905 | −1.4835 | −1.4725 | −1.3005 | −1.7765 |
| CE9PG | 0.939 | 1.05 | 1.027 | 0.134 | 0.366 | 0.821 | 1.597 | 0.905 | −0.276 | −1.024 | 0.306 | −0.471 | −0.155 | −0.373 | −0.168 |
| CELSR1 | 0.734 | 0.764 | 0.652 | 0.88 | 0.134 | 0.528 | 2.444 | 0.265 | −0.224 | −0.737 | 0.111 | −0.836 | −0.586 | −0.227 | −0.538 |
| CENPA | 1.449 | 1.014 | 2.18 | 1.149 | −0.373 | 0.714 | 3.133 | 0.901 | −1.315 | −1.513 | 0.076 | −1.921 | −1.389 | −0.587 | −1.909 |
| CENPE | −0.18 | −1.098 | 0.131 | −0.326 | 0.018 | −0.363 | −0.436 | 0.187 | 0.341 | 0.575 | 0.807 | −0.564 | 1.169 | 0.435 | −0.207 |
| CENPF | −1.3275 | −0.0225 | −0.7195 | −0.0045 | −0.3305 | 0.1005 | 0.4895 | −0.1715 | 0.8595 | 0.2635 | 0.6435 | 0.2745 | 0.4345 | 0.0825 | 0.1875 |
| CENTG3 | 0.8945 | −0.5155 | 0.1775 | −0.3965 | 0.3915 | 1.0125 | −0.0255 | −1.2905 | −0.8445 | −0.6775 | −0.9075 | −1.0045 | −0.5975 | −0.5835 | −0.6025 |
| CES2 | −1.64 | 0.414 | −0.251 | −0.713 | −0.952 | −1.138 | −0.156 | −0.333 | 0.37 | 0.134 | 0.423 | 0.08 | −0.182 | 0.778 | 0.698 |
| CETN3 | 1.7065 | 0.7795 | 0.5435 | 0.4335 | 1.0815 | 0.6685 | 0.9465 | 0.7385 | −1.2045 | −0.5915 | −0.7655 | −0.4155 | −1.2985 | −0.8975 | −1.0145 |
| CGA | 0.6565 | 1.1455 | 1.2885 | 0.3315 | 0.8955 | 0.0695 | 0.9225 | −0.5835 | −0.1555 | 0.3395 | −0.1876 | 0.0955 | −0.3855 | 0.2815 | −0.2885 |
| CGI-12 | −0.3375 | −1.3685 | −0.3265 | −1.6435 | −0.5805 | −1.5785 | −0.1465 | −0.9485 | −0.4675 | −0.9065 | 0.3995 | −0.7395 | −0.8605 | −1.1565 | −0.9205 |
| CGI-49 | −0.974 | −1.691 | −0.429 | −0.309 | 0.823 | 0.298 | −1.314 | −0.808 | −0.99 | −0.871 | 0.016 | −0.718 | −1.664 | −0.805 | −1.015 |
| CGI-4 [symbol] | 0.144 | −1.539 | 0.084 | 0.69 | 0.113 | −1.025 | −0.764 | 0.46 | −0.451 | 0.067 | −0.546 | 0.889 | −0.065 | 1.125 | 1.403 |
| CGN | 0.113 | 1.473 | 1.02 | 1.389 | 0.693 | 0.684 | 1.083 | 0.452 | −1.042 | −0.924 | 0.052 | 0.843 | −0.077 | 0.288 | −0.137 |
| CGNL1 | 1.0355 | 1.9825 | 0.6275 | 0.2825 | 0.5045 | 0.8435 | 0.3055 | 0.3475 | −0.8335 | −0.8745 | 0.4635 | 0.2895 | 0.0815 | −0.2115 | −1.1345 |
| CHAF1B | −0.545 | 0.214 | −0.12 | 0.602 | −0.084 | 0.763 | −0.205 | −0.389 | −0.572 | −0.622 | −0.083 | −0.176 | −0.915 | −0.57 | −0.527 |
| CHC1 | −2.188 | −0.557 | −0.856 | −0.976 | −0.254 | 0.011 | −0.183 | −0.578 | −0.591 | −0.888 | 0 | 0.109 | −0.936 | −0.808 | −0.746 |
| CHCHD2 | 1.178 | 2.471 | 2.187 | 0.845 | 4.006 | 2.324 | 0.894 | 0.053 | 0.669 | −0.12 | 1.728 | 0.318 | 0.378 | −0.53 | 0.72 |
| CHCH05 | 0.62 | 2.096 | 0.011 | 2.854 | 4.567 | 2.228 | 1.34 | 1.183 | 0.611 | 0.431 | 1.036 | 2.489 | −0.09 | 2.147 | 0.728 |
| CHI3L1 | −1.017 | −0.349 | 0.292 | −0.254 | 0.746 | 1.003 | 0.212 | −0.812 | −0.117 | 0.074 | 0.272 | 0.286 | 0.552 | 1.489 | 0.993 |
| CHI3L2 | 1.227 | 0.449 | 0.729 | 0.316 | 0.301 | 0.808 | 0.788 | 0.781 | 0.527 | 0.456 | 0.223 | −0.152 | 0.852 | −0.098 | 0.108 |
| CHKA | 0.096 | −0.466 | −0.379 | 0.664 | 1.676 | 1.165 | 1.252 | −0.164 | 0.227 | −0.391 | 0.009 | 0.197 | 0.597 | −0.255 | −0.048 |
| CHCDL | 0.4435 | 0.4795 | 2.0395 | 0.1185 | −0.3745 | −0.2075 | −0.0235 | 0.7765 | 1.1565 | 0.7305 | 0.6825 | 0.0805 | 0.7095 | 0.1835 | 0.1375 |
| CHRDL2 | −0.735 | −0.488 | −0.12 | −0.077 | 0.581 | 0.124 | 0.208 | −0.757 | −0.334 | −0.15 | −0.192 | 0.303 | −0.276 | −0.741 | −0.367 |
| CHST2 | 0.0765 | −0.4145 | 0.0425 | 0.0505 | 0.6665 | 0.7955 | −0.6365 | 0.0205 | −0.3385 | 0.2755 | 0.7675 | −1.8775 | 0.0275 | −0.1925 | −0.2855 |
| CHURC1 | −1.514 | −0.699 | −0.861 | −0.335 | 0.043 | −1.055 | −1.098 | −0.951 | 0.445 | 1.534 | 0.513 | 0.59 | 0.013 | 1.375 | 1.526 |
| CIR | 0.1235 | 0.4025 | 0.1165 | 0.3425 | 0.4205 | −0.0635 | 0.7055 | −0.0455 | −0.2185 | −0.0055 | 0.4165 | 0.6255 | −1.0145 | −0.4445 | −0.4695 |
| CIRBP | 0.1335 | −0.0065 | 0.4135 | 0.5835 | 0.4875 | 0.0065 | 0.1795 | −0.5715 | 0.1865 | 0.2635 | 0.1855 | −0.2085 | −1.2505 | −0.5165 | −0.5285 |
| CKLF | 1.2875 | 0.2285 | 1.0785 | 1.0415 | 0.9435 | 1.2105 | 1.3755 | 0.8855 | 0.8555 | 0.8885 | 1.5785 | 1.7105 | 1.4125 | 1.9305 | 2.0955 |
| CKLFSF6 | −2.4225 | −0.3285 | 0.7716 | −0.1145 | −0.6285 | 0.0515 | −0.9335 | −1.1325 | −0.9195 | −1.1455 | 0.5145 | 0.3435 | 0.0115 | 0.4225 | 0.0295 |
| CKLFSF7 | 1.583 | 0.822 | 1.652 | 1.273 | 1.041 | 0.591 | 0.97 | −0.131 | −0.47 | −0.168 | 0.081 | 0.346 | −0.685 | −0.275 | −0.483 |
| CKMT1 | 1.446 | 1.114 | 0.95 | 0.263 | −0.208 | 1.69 | 2.806 | −0.813 | −1.226 | −1.299 | 0.669 | −0.303 | −2.151 | −0.378 | −1.928 |
| CKS1B | 0.332 | 0.554 | 0.914 | 0.068 | 0.385 | −0.423 | 1.107 | −0.801 | −0.32 | −0.669 | 0.097 | 0.682 | −0.818 | −0.512 | −0.655 |
| CKS2 | 0.9805 | 0.4835 | 0.0345 | 0.5825 | 2.3345 | 3.5575 | 1.7275 | 1.1385 | −0.0515 | −0.3065 | −1.4315 | 0.7085 | −0.8835 | 0.0015 | 0.1585 |
| CL640 | 1.5885 | −1.3165 | 0.8315 | −1.0855 | 0.8105 | 0.6905 | −2.0925 | 1.1435 | −0.6175 | −0.6305 | 0.2395 | 0.2415 | −0.5795 | 0.5375 | −0.7045 |
| CLDN23 | 0.1345 | −1.3455 | −0.6795 | −0.4755 | 0.5476 | 0.1416 | −1.8745 | 0.2295 | 0.3375 | 0.1695 | 0.3985 | −0.2645 | 0.4705 | 0.0385 | −0.3315 |
| CLDN3 | −0.6385 | 0.1595 | −0.6345 | 0.5725 | −0.3505 | −0.4945 | −0.4625 | −0.8795 | 0.2085 | −0.0615 | 0.1875 | 1.3305 | −0.1745 | 0.9815 | 1.2915 |
| CLDN4 | −0.692 | −0.891 | −1.346 | −1.329 | −1.129 | −0.791 | −0.348 | 0.58 | −0.658 | −1.523 | −1.142 | −1.288 | −0.522 | −0.961 | −1.241 |
| CLDN8 | 0.6925 | −0.5775 | −0.3575 | −0.0275 | 0.2395 | −0.8135 | 2.4535 | 0.4875 | −0.2775 | 0.0225 | −0.0305 | 1.5135 | −0.4725 | −0.0025 | −0.0955 |
| CLGN | 8.5595 | −0.2095 | 0.4585 | 0.0475 | 0.3035 | 1.2105 | 0.6395 | 0.1305 | −0.2675 | −0.1845 | −0.0035 | 0.2685 | 0.1945 | 0.1135 | −0.4935 |
| CLIC6 | −0.279 | 0.813 | 1.33 | 0.168 | −0.089 | 0.661 | 0.149 | −1.022 | −0.679 | −0.652 | −1.167 | −1.011 | −0.858 | −0.826 | −0.557 |
| CLNS1A | −1.6785 | −2.0735 | 0.9405 | −1.1425 | 0.1035 | 1.9495 | −0.7785 | 0.5295 | 1.3525 | 1.5555 | 1.7565 | 0.9276 | −0.0535 | 0.6925 | 1.8955 |
| CLTC | 2.135 | 0.933 | 0 | −0.228 | 0.692 | 0.512 | −0.307 | 2.434 | −0.811 | −0.351 | −0.884 | 0.401 | −1.47 | −0.007 | 0.195 |
| CLU | 1.58 | 1 | 1.555 | 0.752 | 1.04 | 1.694 | 0.91 | −0.118 | −0.308 | −0.289 | −0.376 | −8.48 | −0.88 | −0.374 | −0.42 |
| CMAS | 0.1855 | 0.0295 | 0.1935 | 0.2845 | 0.3135 | −0.0205 | 0.2705 | −1.5155 | −0.4155 | −0.7055 | −0.7205 | 0.7785 | −1.5395 | −0.8155 | −0.8965 |
| CML65 | 1.022 | 0.091 | 0.163 | 0.474 | 0.442 | 0.02 | 0.329 | 0.021 | −0.129 | 0.122 | 0.328 | −0.755 | −0.88 | 0.188 | −0.298 |
| CNIH | 0.188 | −0.429 | −8.242 | −0.061 | −0.102 | −0.245 | −0.682 | 0.585 | −0.475 | −0.102 | −0.038 | −0.813 | 0.361 | 0.232 | −0.185 |
| CNOT10 | 0.8495 | −0.3976 | 0.6825 | 0.5805 | 0.5135 | 1.1755 | 0.3435 | 0.1775 | 0.2045 | 0.5695 | 0.0115 | −0.0805 | −0.0395 | 0.3785 | −0.0235 |
| CNOT2 | 1.172 | 1.217 | 0.038 | 0.19 | 0.257 | −0.157 | −0.4 | −0.002 | 0.265 | 0.217 | 0.294 | 0.366 | −0.205 | 0.026 | −0.173 |
| CNOT7 | 1.834 | −1.767 | −2.105 | −2.068 | −1.296 | −2.485 | −2.288 | −0.051 | −1.315 | −1.884 | 0.277 | −1.182 | −1.288 | −1.644 | −1.72 |
| CNP | −0.5195 | −0.5895 | −0.4865 | −0.4276 | −0.5915 | 0.3045 | −0.7945 | −0.9785 | −0.1555 | −0.1935 | 0.1705 | 0.3775 | −0.0815 | 0.8685 | 0.2045 |
| CNTNAP2 | 0.174 | −0.403 | −0.101 | 0.071 | −0.635 | 2.312 | −0.108 | 0.445 | −0.352 | −0.347 | −0.453 | 0.075 | −0.274 | 0.192 | 0.308 |
| COASY | 0.8575 | 0.3535 | 2.0325 | 1.2845 | −0.1315 | 1.1585 | 2.6705 | 1.5175 | 0.0135 | 0.6765 | 0.6116 | 0.1795 | 0.4085 | 1.9855 | 0.6975 |
| COL11A2 | −0.588 | −1.254 | −1.149 | −0.434 | −1.195 | −0.337 | −2.012 | −1.891 | −0.181 | 0.233 | −0.038 | 0.126 | −0.148 | 0.794 | −0.665 |
| COL27A1 | 1.88 | −0.38 | −0.457 | 0.437 | 0.007 | 1.331 | 0.808 | −0.024 | 0.802 | 0.853 | 0.496 | 0.287 | 0.847 | −0.035 | −0.56 |
| COL4A5 | 0.1995 | 2.0285 | −1.3525 | −0.3045 | 1.6925 | 5.6725 | 4.7635 | 3.9005 | −0.1165 | 0.9765 | −0.8835 | 0.7695 | 0.2015 | 0.3935 | −0.7335 |
| COL8A1 | 0.109 | −0.437 | 0.322 | −0.018 | 0.718 | −0.738 | −0.485 | −0.702 | −0.929 | −0.594 | −1.423 | 0.821 | −1.702 | −0.251 | −0.259 |
| COL8A3 | 0.565 | −0.173 | 1.013 | −0.095 | 1.132 | 0.351 | 0.205 | −0.365 | −0.472 | −0.735 | 0.004 | 0.08 | −1.087 | −0.745 | −0.57 |
| COMMD1 [symbol] | −0.1595 | 0.0885 | −0.3385 | −0.1925 | 0.0535 | −0.1395 | −0.0676 | 0.1005 | 0.0605 | 0.1335 | 0.1725 | 0.0015 | 0.7805 | −0.1215 | 0.1755 |
| COMMD5 | −0.8125 | −0.0055 | −0.1655 | −0.0635 | −0.6015 | −0.3115 | −0.1665 | 0.2545 | 1.4335 | 0.3895 | 1.2945 | 0.0405 | 1.2055 | −0.2035 | 0.5255 |
| COPS7B | 0.602 | 1.896 | 0.533 | 0.575 | −0.05 | −0.261 | −0.301 | 0.472 | 0.158 | −0.424 | 0.189 | 0.028 | 1.135 | 0.282 | −0.187 |
| CORO6 | 0.4675 | −0.4035 | 0.3725 | −0.4785 | −0.0435 | −0.1895 | 0.0835 | −0.3655 | −0.6605 | −1.0835 | −0.5745 | −0.2355 | −0.1865 | −0.9125 | −0.9335 |
| CORT | 0.295 | 0.58 | 0.75 | 0.256 | 0.623 | 0.264 | 1.001 | −0.061 | −0.549 | −0.849 | −0.123 | 0.55 | −0.946 | 0.027 | −0.331 |
| COX17 | 0.18 | 0.176 | 0.838 | 0.213 | 0.653 | 0.226 | 0.86 | −0.092 | −0.718 | −0.845 | −0.263 | 0.822 | −0.837 | 0.019 | −0.524 |
| COX5A | 0.828 | −0.381 | 0.799 | 0.004 | −0.39 | −0.004 | 0.547 | −0.048 | −0.737 | −1.07 | −0.338 | −0.568 | −0.689 | −0.935 | −0.808 |
| COX6A | −0.0285 | −1.3575 | −0.6445 | −0.8645 | 0.1795 | 0.4245 | −0.4555 | −0.4775 | −1.5935 | −2.2015 | −0.5685 | −0.7175 | −0.9195 | −1.7645 | −1.6365 |
| COX8A1 | −0.0045 | 0.3175 | −0.5355 | 0.3495 | 0.3795 | 1.0295 | 0.3355 | −0.5855 | −0.4065 | −0.7915 | −0.2125 | −0.0825 | −0.5565 | −0.7425 | −0.8815 |
| COX8C | −1.3365 | −0.4845 | 1.4845 | 2.7485 | 2.8525 | 3.3445 | 6.1175 | 0.2885 | 1.7115 | 0.1865 | 3.0515 | −1.0225 | 2.7215 | 0.6915 | −0.3375 |
| COX7A2 | −1.579 | −0.018 | −1.539 | 0.308 | 0.172 | 2.05 | 0.887 | 0.012 | 0.615 | 1.017 | 1.048 | 1.563 | 1.821 | 2.128 | 1.485 |
| CP | −0.4965 | −0.2925 | −0.3215 | −0.0895 | −0.4805 | −0.7295 | 1.3135 | −0.0235 | 3.3455 | 0.3635 | 2.0075 | −0.6275 | 0.8295 | −0.7885 | −1.0815 |
| CPAMD0 | −0.601 | −0.726 | −0.468 | −0.509 | 0.013 | 0.445 | −1.238 | −0.18 | −0.129 | 0.078 | −0.572 | −1.776 | −1.549 | 0.384 | 0.514 |
| CPB1 | −1.4565 | −0.1355 | −0.1135 | −0.4295 | −0.7735 | −0.4085 | −0.6995 | −0.4935 | 0.2635 | −0.0585 | −0.2815 | −1.7685 | −1.6245 | −0.6405 | −0.1925 |
| CPEB3 | 8.886 | 0.679 | 0.215 | 0.022 | 0.393 | 0.099 | −0.891 | 0.313 | −0.228 | −0.391 | 0.18 | −0.754 | 0.33 | 0.091 | −0.407 |
| CPEB4 | 1.535 | 1.889 | 2.267 | 1.002 | 1.211 | 0.146 | −0.416 | 1.058 | 0.383 | 0.116 | 0.188 | 1.786 | 1.082 | 0.945 | 0.481 |
| CPSF3 | −1.7355 | −1.0575 | −1.6416 | −1.1015 | −1.9855 | −1.9085 | −2.5245 | −2.1165 | −1.7465 | −2.0235 | −0.6685 | −0.7615 | −1.2055 | −0.1335 | −1.1105 |
| CRABP1 | −2.22 | −1.419 | −1.843 | −1.335 | −0.942 | −2.332 | −2.588 | −2.005 | −0.5 | −0.964 | −0.556 | −2.114 | −1.628 | −1.688 | −0.687 |
| CREB3L4 | 1.845 | −0.23 | −0.938 | −0.163 | 1.9 | 1.444 | −0.298 | 0.318 | 0.35 | −0.264 | −0.02 | −0.501 | 1.198 | −0.152 | −0.191 |
| CRIP1 | −0.653 | −0.695 | −0.868 | −0.181 | −0.837 | −0.452 | −1.273 | −0.71 | 0.511 | 0.9 | 0.396 | 0.573 | −0.817 | −0.368 | 0.306 |
| CRYBB2 | 0.407 | 0.161 | 0.41 | 0.772 | 0.442 | −0.279 | −0.916 | −0.709 | −0.605 | −0.348 | −1.223 | −0.332 | −1.389 | −0.471 | −0.454 |
| CRYL1 | 1.964 | 0.045 | −0.372 | 0.272 | 1.735 | 1.142 | −0.033 | 1.078 | 1.434 | 1.052 | 0.891 | 0.478 | −0.685 | 0.73 | 0.716 |
| CRYZ | 1.8125 | 1.0805 | 0.7855 | 0.0035 | 0.7505 | 0.7555 | 1.9765 | 0.3335 | −0.5435 | −0.2725 | −0.0645 | −0.9975 | −0.0355 | −0.0925 | −0.3905 |
| CSDA | −0.0355 | 1.8335 | 0.0545 | 0.1275 | 0.3565 | −0.0065 | 0.0635 | 0.4825 | 0.3885 | 0.3535 | 0.3845 | 0.0255 | 0.1005 | 0.0615 | 0.1655 |
| CSE1L | 0.2295 | 0.0905 | −0.6776 | −0.0126 | 0.1185 | 0.0735 | −1.1595 | −0.6305 | 1.8895 | 1.8255 | 0.8765 | 1.5745 | −1.1105 | 1.7285 | 1.5845 |
| CSK | 2.2975 | 0.8765 | 1.0085 | 1.2845 | 1.1525 | 0.5675 | 1.3795 | 3.2615 | 1.0435 | 1.7065 | 0.1205 | 0.0785 | 0.2815 | −0.4795 | −0.0735 |
| CSRP1 | −0.5995 | −0.8516 | 0.1195 | −0.3635 | 2.1015 | 2.5765 | −0.9985 | −1.2295 | −1.2675 | −2.1055 | −0.0925 | −0.5535 | −0.9735 | −1.1675 | −1.6735 |
| CSRP2 | 0.25 | 0.14 | 1.085 | 0.896 | 0.713 | −0.168 | −0.258 | 0.088 | −0.293 | −0.786 | 1.203 | −0.299 | −0.591 | −0.38 | −0.197 |
| CST5 | −0.5685 | 0.3475 | 6.9195 | 0.2145 | −0.3275 | 4.8385 | −1.1705 | 1.0865 | −0.0315 | 0.2365 | −0.3835 | 4.0495 | 2.0655 | 0.6535 | 0.4225 |
| CSTB | 0.131 | −0.618 | −0.994 | −0.389 | −0.42 | 0.61 | −1.181 | −0.252 | −0.184 | 0.275 | 0.18 | −0.566 | −0.198 | 0.362 | −0.173 |
| CTAG1B | −0.1465 | −0.2255 | −0.1935 | 0.2315 | −0.1975 | −0.2315 | −0.8435 | −0.4965 | −0.2775 | −0.0175 | −0.0335 | 0.0975 | −0.8285 | −0.0405 | 0.2745 |
| CTBP2 | 0.608 | 0.496 | 0.076 | 0 | 0.021 | 1.37 | 1.525 | −0.384 | 0.924 | 1.288 | 0.121 | −0.408 | −0.088 | 0.468 | 0.275 |
| CTNNA1 | −1.648 | −0.716 | −1.373 | −0.291 | −1.816 | −1.676 | −1.836 | −0.681 | −0.708 | −0.903 | 0.693 | 0.233 | 0.287 | 0.104 | −0.042 |
| CTNNAL1 | 1.4005 | 0.7955 | 1.2385 | 0.5185 | 1.3646 | 0.2965 | 0.9955 | 1.5545 | −0.2455 | −0.4895 | −0.1206 | 0.3985 | −1.5565 | −0.5365 | −0.8775 |
| CTNND2 | 0.478 | 0.425 | 2.334 | 1.384 | 0.772 | 2.377 | 2.004 | 0.35 | 0.082 | 0.374 | 0.921 | −0.203 | 0.512 | −1.304 | −1.088 |
| CTPS | 0.694 | 1.542 | 3.296 | 1.181 | 2.248 | 0.748 | 0.086 | 1.065 | −0.617 | −0.037 | 0.546 | −0.109 | −0.354 | −0.672 | −0.157 |
| CTSL2 | −0.1975 | 1.7965 | −0.2435 | −0.6875 | 0.6315 | 0.8535 | 0.1085 | −0.7295 | −0.3805 | −0.7975 | 0.4735 | −0.2115 | −1.2645 | −0.7045 | −0.6805 |
| CTSS | 0.317 | 0.076 | 0.36 | 1.84 | 0.879 | 0.74 | 1.517 | 0.23 | 2.2 | 3.28 | 2.32 | 2.85 | 3.888 | 2.54 | 2.426 |
| CTTN | −0.9615 | 1.6876 | 3.0105 | 1.8925 | 2.6855 | 1.5795 | 1.6155 | 1.1855 | −2.7405 | −1.0315 | −0.9975 | −2.2385 | −2.2975 | −2.1075 | −1.5665 |
| CX3CL1 | −0.3225 | −0.5155 | 0.0235 | −0.7165 | −0.0975 | 0.1985 | 0.1025 | −0.2595 | −0.7645 | −1.2775 | −0.7855 | −1.0265 | −2.1605 | −1.3745 | −1.6375 |
| CXCL9 | 0.0345 | −1.7215 | −1.4135 | −0.9205 | −1.8485 | −2.2295 | −1.2345 | −0.8015 | −0.7835 | −1.3155 | 0.1095 | −0.8075 | −0.8565 | 0.4525 | 0.1205 |
| CXorf39 | −0.876 | −2.911 | −1.548 | −1.482 | −1.108 | −2.449 | −2.507 | −1.028 | −0.189 | −1.171 | −0.079 | 0.271 | −1.691 | −1.14 | −0.748 |
| CXXC4 | −0.682 | −0.183 | −0.842 | −1.014 | −0.814 | 0.988 | −1.113 | −0.64 | −1.185 | −0.74 | −0.528 | −0.274 | −0.859 | −0.522 | −0.557 |
| CYB5 | 0.7145 | 0.2075 | 1.4975 | 0.1585 | 0.7245 | 0.3416 | 1.0675 | −0.1285 | −0.6335 | −0.9615 | −0.2535 | 0.3285 | −1.0675 | −0.4115 | −0.6415 |
| CYB561 | −0.825 | −0.87 | −0.522 | −1.192 | 0.881 | −0.261 | 0.036 | −0.537 | −0.321 | −0.254 | 0.518 | −0.305 | −0.279 | 0.155 | −0.044 |
| CYC1 | 0.085 | −0.04 | 0.079 | 0.002 | 0.115 | −0.105 | 0.034 | −0.119 | 0.564 | 0.446 | 0.544 | 0.216 | 1.195 | 0.175 | 0.225 |
| CYHR1 | −0.5095 | −1.0905 | 0.0875 | −0.2505 | −0.8325 | −1.6865 | −0.4995 | −1.3455 | 2.6035 | 0.8095 | 1.8475 | −0.9205 | 1.7605 | −0.4455 | −0.1245 |
| CYP2A13 | 0.675 | −0.148 | 0.218 | 0.427 | −0.101 | −0.358 | −0.68 | −0.038 | 0.675 | 0.651 | 0.449 | 0.183 | 0.235 | 0.327 | −0.041 |
| CYP4X1 | −2.3875 | −1.7345 | −1.8955 | −1.4555 | −1.8985 | −2.5075 | −2.3255 | −2.4375 | −0.5555 | −0.2905 | −1.0455 | −0.8495 | −0.9765 | −0.8845 | −1.1395 |
| D15Wsu75 | −0.7395 | −1.0795 | −1.3875 | −1.2535 | −0.8295 | −1.2005 | −1.1385 | −0.8815 | −0.1255 | 0.4015 | −0.5475 | −0.6465 | −0.1485 | −0.0055 | −0.3315 |
| DACH1 | −1.741 | −1.877 | −2.317 | −1.719 | −1.422 | −2.253 | −1.454 | −1.702 | −0.291 | 0.097 | −0.847 | −0.845 | −1.864 | −1.37 | −1.224 |
| DACH1 | 1.328 | 0.179 | 0.968 | 0.308 | 0.526 | −0.468 | −0.503 | 0.198 | −0.817 | −0.87 | −0.458 | −0.403 | −0.476 | −0.505 | −0.722 |
| DACH1 | 0.566 | 1.088 | 1.741 | 1.248 | 1.705 | 1.416 | 0.831 | 0.083 | −0.384 | 0.599 | −0.028 | −0.326 | −0.224 | −0.026 | −0.143 |
| DAP3 | 0.2195 | 0.1965 | 0.4355 | 0.3155 | 0.3915 | 0.0675 | −0.2835 | −0.0485 | −0.5095 | −0.1565 | −0.5165 | −0.0495 | −0.2035 | −0.4775 | −0.2125 |
| DAPP1 | −0.200 | 0.009 | −0.274 | 0.587 | 0.888 | 0.885 | −0.031 | −0.568 | −0.158 | −0.898 | 0.348 | 0.832 | −0.778 | 0.002 | 0.312 |
| DAZAP2 | 0.351 | 0.01 | −0.74 | −0.046 | 0.408 | −0.063 | 0.387 | 0.048 | 0.378 | 0.576 | −0.314 | 0.271 | −0.424 | −0.557 | −0.134 |
| DBI | −0.2575 | 0.2705 | 1.3095 | 0.2605 | 0.5035 | 0.2815 | 1.5385 | 0.5015 | −0.1235 | −0.4505 | −0.6885 | 0.4245 | −0.0915 | −0.2025 | −0.6075 |
| D9N1 | 0.996 | 0.579 | 0.01 | 0.437 | 1.061 | 1.018 | 1.026 | 0.027 | −0.329 | −0.629 | −0.168 | 0.682 | −1.428 | 0.08 | −0.34 |
| DC13 | 1.5005 | 0.1835 | 1.0115 | 1.4855 | 0.6976 | 0.9316 | 0.7875 | 0.3695 | −0.2745 | 0.0895 | −0.5155 | −0.2025 | −1.1555 | −0.2885 | −0.2365 |
| DCPS | −0.0075 | −0.4016 | −0.1035 | −0.2115 | −0.4145 | −0.2295 | −0.2215 | −0.3405 | −0.2075 | 0.5045 | −0.1275 | 0.2875 | 0.0885 | 0.0695 | 0.0565 |
| DDHD2 | −0.2725 | 0.7935 | 0.2305 | 0.0375 | 0.1105 | 0.0045 | 1.3305 | 0.6495 | −0.4895 | −0.4125 | 0.2355 | −0.0105 | −0.6805 | −0.1885 | −0.6435 |
| D0X17 | −0.4775 | 0.6715 | 0.1445 | 0.1915 | 0.6235 | 0.8025 | 1.0355 | −0.3376 | −0.0655 | −0.1385 | −0.0615 | 0.2115 | −0.8525 | −0.0055 | −0.4415 |
| DDX39 | 0.34 | 1.736 | 1.018 | 1.311 | 1.651 | 1.144 | 0.43 | −0.791 | −0.358 | −0.351 | −0.29 | −2.111 | −0.756 | −0.989 | −1.656 |
| DDX48 | −0.1205 | 0.5665 | 0.8895 | 0.8855 | 1.0995 | 0.3985 | −0.0235 | 0.7795 | −0.5495 | 0.4945 | 0.6225 | −0.7275 | 0.1195 | 0.3235 | −0.4725 |
| DDX58 | 1.2835 | 1.1165 | 1.1465 | 2.1865 | 1.1515 | 0.8785 | 1.8525 | 1.0055 | −0.3175 | 0.2715 | −0.8945 | −1.0805 | −0.0855 | 0.1165 | 0.0265 |
| DEF6 | 0.8 | −0.67 | −0.714 | 0.037 | 0.349 | −0.348 | −0.195 | 0.745 | −0.484 | −0.395 | 0.032 | 0.12 | −1.172 | −0.078 | −0.299 |
| DEK | 0.411 | −0.295 | −0.529 | −0.266 | 1.317 | −0.299 | −0.087 | −0.876 | −0.47 | −0.05 | −0.938 | 0.044 | −1.23 | −0.984 | −0.784 |
| DHCR7 | −0.037 | 0.805 | −0.41 | 0.33 | 0.165 | 1.472 | 1.554 | 0.682 | 1.091 | 0.24 | 0.503 | 0.684 | −0.325 | 0.818 | 1.145 |
| DHRS7 | −2.8405 | −0.7575 | −0.4925 | −0.3745 | −2.0435 | −0.9535 | 0.6155 | 0.5625 | −0.0916 | 0.6705 | 0.4716 | 1.1105 | 0.0875 | 1.1145 | 0.7825 |
| DHX57 | −1.6305 | −2.1175 | −2.0575 | −1.9215 | −1.1885 | −1.9955 | −1.6225 | −1.6035 | −0.3316 | −1.3205 | −0.3865 | 0.0345 | 0.8345 | 0.7085 | −1.0085 |
| DIRC1 | −0.502 | −0.111 | −0.301 | −0.058 | −0.835 | 0.481 | −0.105 | −1.032 | 0.726 | 0.638 | 0.722 | 0.496 | 0.799 | 0.882 | 0.553 |
| dJ222E13 | 3.829 | −0.331 | 2.962 | 0.233 | 1.099 | 0.986 | −0.433 | 1.321 | −0.286 | −0.321 | −0.072 | 0.845 | −0.91 | 0.51 | −0.058 |
| DKFZP434 | 0.329 | 1.163 | 0.071 | 0.009 | 0.832 | −0.136 | −0.507 | 0.133 | −0.807 | −0.661 | −0.452 | 0.033 | −0.498 | −0.582 | −0.491 |
| DKFZp434 | −0.0295 | 0.4625 | 0.5725 | 0.4715 | −0.5485 | 0.5435 | 1.1225 | 1.9605 | −0.7855 | −0.4755 | 0.2485 | −0.5855 | 0.4045 | 0.1255 | −0.5235 |
| DKFZP434 | 0.008 | 0.228 | 0.003 | −0.193 | 0.052 | 0.415 | −0.041 | −0.41 | 0.019 | −0.357 | 0.123 | 0.328 | 0.157 | −0.628 | 0.046 |
| DKFZp434 | 0.0185 | 0.5385 | −0.0315 | −0.1595 | 0.1415 | −0.2675 | −1.0345 | 0.0465 | −0.1115 | −0.2885 | −0.0025 | −0.3205 | −0.1655 | −0.8795 | −0.4445 |
| DKFZP564 | 1.3835 | 0.0045 | −0.5865 | −0.0025 | 0.0855 | 0.2395 | −0.3015 | 0.4175 | −0.9345 | −0.6435 | −0.4155 | 0.4305 | 0.0065 | −0.3015 | −0.4415 |
| DKFZp564 | 1.657 | 0.746 | 0.878 | 0.21 | 1.095 | 0.76 | 0.478 | 0.148 | −0.448 | −1.019 | −0.721 | −0.893 | −1.158 | −1.133 | −1.276 |
| DKFZP564 | 1.1825 | −0.2675 | 0.1425 | 0.2735 | 0.8915 | 0.2435 | −0.0825 | −0.9415 | 0.1045 | 0.2475 | −0.1365 | −1.2995 | −0.5595 | −0.0405 | −0.2645 |
| DKFZP564 | 0.736 | −0.521 | 0.144 | 0.168 | 0.661 | 0.598 | 1.803 | −0.767 | 0 | −0.067 | −0.171 | 0.502 | −0.318 | −0.214 | −0.282 |
| DKFZP564 | −0.768 | −0.85 | 0.402 | 0.348 | −0.38 | −0.208 | 0.152 | −1.098 | 0.039 | −0.384 | 0.207 | 0.288 | 0.103 | 0.771 | 0.76 |
| DKFZp586 | −1.3115 | −0.3435 | −1.5325 | 1.7035 | −1.2275 | 3.2325 | −0.1835 | −0.4835 | 1.3775 | 3.0295 | 2.4625 | 5.6545 | 2.3805 | 2.2895 | 2.2075 |
| DKFZp761 | 0.06 | 0.105 | 0.509 | 0.159 | 0.037 | 0.022 | 1.164 | 0.228 | 1.47 | 0.656 | 0.874 | 0.012 | 0.298 | −0.115 | 0.08 |
| DLK1 | −1.242 | 0.111 | 0.848 | −0.503 | −0.929 | −0.276 | −0.71 | −2.102 | −0.744 | −1.214 | −1.025 | −0.783 | −1.384 | −1.148 | −0.744 |
| DLX5 | −0.0885 | −0.2945 | −0.6915 | −0.9455 | −0.6135 | −0.1355 | −1.1255 | −0.3145 | −0.6585 | −1.0755 | 0.1475 | −1.4145 | −1.2445 | −0.7905 | −0.6805 |
| DNAJA4 | −2.8755 | −2.5305 | −3.6805 | −1.6745 | −2.2825 | −0.1775 | −3.6255 | −0.4825 | −0.9135 | −1.4985 | −1.4016 | 0.2605 | −0.7465 | 0.8475 | −2.0515 |
| DNAJC1 | 0.201 | −0.024 | −0.972 | 0.674 | 0.681 | 0.681 | −0.377 | −1.36 | −0.394 | −1.545 | −0.33 | −0.291 | −1.273 | −0.842 | −0.708 |
| DNAJC12 | −2.481 | −2.801 | −3.33 | −1.718 | −1.925 | −2.782 | −3.14 | −3.595 | −0.7 | −0.225 | 0.404 | −0.58 | 0.019 | −0.881 | −2.254 |
| DNAJD1 | 1.134 | 0.885 | 1.624 | 1.115 | −0.389 | −0.259 | 1.038 | 0.169 | −0.548 | −0.375 | 0.035 | −0.028 | −0.81 | 0.363 | −0.386 |
| DNALI1 | 0.08 | 1.12 | −0.551 | 0.424 | 0.537 | −0.762 | −0.316 | 0.922 | 0.643 | 0.03 | 0.822 | 0.179 | 0.38 | −0.2 | −0.102 |
| DNMT1 | −0.8165 | 1.8215 | 0.8835 | 1.5085 | 0.3845 | 1.1615 | 1.0315 | −1.1505 | 1.7495 | 2.1085 | 0.5035 | 1.7495 | 1.7835 | 1.7276 | 1.6375 |
| DOX1 | 1.0335 | −0.1215 | −0.5205 | 0.3855 | 1.0005 | 0.1045 | 0.6585 | 0.6795 | −1.0975 | −0.4025 | −0.2365 | −0.4155 | −0.5185 | −0.8825 | −0.5385 |
| DSCR1 | 1.3115 | 0.1755 | −0.5295 | 2.0665 | 0.5795 | 0.9365 | 0.9605 | 0.4375 | −0.3255 | −0.3315 | 0.1655 | −1.2695 | −1.4216 | 0.2495 | −0.9845 |
| DSCR1L2 | 0.3845 | 1.2045 | 0.5915 | −0.0025 | −0.3555 | −0.0435 | −0.0925 | −1.0825 | −0.5445 | −0.5595 | 0.9025 | 0.0345 | −0.4455 | −0.1405 | −0.6225 |
| DSP | 0.6835 | −0.4015 | −0.1745 | 0.2575 | 0.6235 | −0.0055 | −0.5215 | −0.5545 | 0.2285 | −0.8385 | 0.9615 | 0.0635 | 0.0085 | −1.0365 | −0.1955 |
| DTYMK | −0.9065 | −0.2895 | −0.7185 | −0.3465 | −1.0505 | −0.5716 | −1.3435 | −0.7045 | 0.5245 | −0.1885 | 0.0245 | 0.0945 | 0.6475 | 1.1375 | −0.4975 |
| DUSP23 | −1.2925 | −0.7545 | −0.5885 | 0.5565 | −0.2185 | −1.0955 | −1.0895 | −0.3555 | 1.2395 | 0.8755 | 0.5865 | 1.2855 | 0.0595 | 1.7325 | 1.3175 |
| DUSP4 | 0.1565 | −0.4435 | −0.9695 | −0.3185 | −0.8655 | 0.0815 | 0.2785 | −0.5965 | 0.1645 | 0.0525 | 0.1285 | −0.0985 | 0.3465 | −0.1135 | 0.0125 |
| DUSP5 | 0.236 | 1.71 | 1.004 | 0.292 | −0.098 | 0.103 | 0.174 | 0.276 | 0.181 | −0.371 | 0.002 | 0.163 | 0.067 | 0.074 | −0.499 |
| DVL1 | 1.669 | 0.233 | −0.925 | −0.215 | 1.308 | 1.154 | 0.043 | 0.44 | 0.305 | 0.041 | 0.533 | 0.485 | 0.25 | −0.179 | −0.432 |
| DXYS155 [symbol] | 0.656 | 0.714 | 1.38 | 0.544 | 0.89 | 1.214 | 1.007 | −0.58 | −0.308 | −0.785 | −0.257 | −0.68 | −0.723 | −0.507 | −0.588 |
| DYRK4 | 1.0545 | 1.6605 | 2.1515 | 0.7935 | 1.0645 | −0.3915 | 0.3775 | 1.4635 | −0.6545 | −0.7685 | −0.4645 | 0.0105 | −0.5325 | −0.4525 | −0.1915 |
| o(y)2 | −0.23 | 0.305 | −0.063 | −0.077 | 0.162 | −0.215 | 0.625 | −0.167 | 0.168 | −0.071 | 0.484 | 0.259 | −0.152 | −0.053 | 0.042 |
| EAF2 | 1.1835 | 0.7495 | 0.5115 | 1.2195 | 0.6625 | 0.2465 | 0.9455 | 0.5545 | 0.8725 | 0.9775 | 0.2995 | −0.4515 | −0.9325 | −0.4875 | 0.7785 |
| EAP30 | 0.1955 | −0.7775 | −1.5075 | −0.8705 | 1.2675 | 0.4455 | −0.2055 | −1.2476 | 0.5825 | −0.2315 | −0.0135 | 0.9595 | −0.3045 | 0.0335 | 0.8545 |
| ECHDC1 | −0.1715 | −0.7065 | −0.4995 | −0.9126 | −0.6435 | −0.9695 | −0.8125 | 1.3175 | 0.0855 | −0.8145 | 0.2735 | −0.5715 | −0.2805 | −0.2895 | 0.1245 |
| ECHDC3 | −0.5005 | −0.7785 | −1.7585 | −0.6915 | −0.2885 | 1.1775 | 0.6625 | 0.2515 | −0.5935 | −0.0735 | −0.4785 | −0.4775 | −0.0295 | −0.2285 | −0.3665 |
| EEF1A2 | 0.626 | 0.825 | 0.037 | 0.521 | −0.021 | −0.083 | −0.57 | −0.206 | −0.851 | −0.619 | −0.005 | 0.05 | −0.824 | −0.132 | −0.546 |
| EFHC1 | −0.8245 | 0.0155 | −3.4185 | −0.1585 | 0.1785 | 0.6845 | 0.8185 | −0.3975 | 0.2265 | 0.1555 | 0.4855 | 0.3745 | −0.8255 | 0.2835 | −0.4075 |
| EFNA4 | 0.5715 | 0.5845 | 0.5025 | 0.1995 | 1.4945 | 0.4715 | 0.7575 | 0.0705 | 0.0545 | −0.4865 | −0.2525 | −0.9995 | 1.1345 | −0.3905 | −0.2105 |
| EFS | 0.4575 | −0.3675 | −0.0805 | 0.0945 | 0.6215 | 0.4465 | 0.0355 | −0.3335 | −0.0005 | 0.2125 | 0.0005 | −0.5425 | −0.6225 | −0.2445 | −0.1275 |
| EIF2C2 | 1.2985 | −0.2445 | 0.7945 | 1.3205 | −0.1735 | 0.5385 | 0.0795 | 0.4705 | 0.4055 | −0.1515 | 0.8025 | 0.1485 | 0.0515 | 0.3585 | −0.3375 |
| EIF2C4 | −0.185 | −0.002 | 0.148 | −0.07 | −0.224 | 0.082 | −0.669 | 0.402 | 0.454 | 0.133 | 0.424 | −0.306 | −0.617 | −0.007 | −0.231 |
| EIF4EBP1 | 0.2245 | −0.4325 | −0.0375 | −0.2525 | −0.1615 | −0.0055 | 0.1955 | −0.3845 | 0.0055 | −0.2805 | −0.2755 | −1.1385 | −1.0185 | −0.1175 | −0.1445 |
| EIF4G1 | 1.564 | 0.284 | 1.642 | 0.497 | 1.157 | 0.07 | 0.186 | 0.471 | −0.425 | 0.281 | 0.182 | 1.333 | −0.528 | −0.302 | 0.309 |
| EIF4G3 | −0.999 | 1.553 | 1.432 | 0.235 | 0.164 | 0.518 | 0.12 | 0.181 | 1.112 | −0.552 | −0.195 | −1.532 | −1.361 | −0.124 | −0.527 |
| ELAC1 | −0.22 | −0.422 | −0.108 | −0.088 | −0.122 | −0.863 | −0.592 | −0.904 | −1.342 | −1.216 | −0.43 | 0.773 | −1.298 | −0.018 | −0.284 |
| ELL2 | −1.4115 | −0.8545 | −0.5885 | −0.9545 | −0.6975 | −0.7485 | −1.8195 | −2.1965 | 0.2715 | −0.8825 | −0.4635 | −0.0045 | −1.2585 | 0.1465 | −0.0845 |
| ELL3 | 1.8635 | 2.5125 | 2.4745 | 1.7525 | 3.0655 | 3.9315 | 3.5085 | 0.2845 | 0.7605 | 0.0595 | 0.1315 | 0.8365 | −0.4965 | −0.1145 | 0.0965 |
| ELOVL5 | 0.6105 | −0.5455 | −0.9115 | −0.1485 | −0.8525 | −0.2115 | 0.0625 | −0.7875 | −0.8155 | 0.3045 | −1.3445 | −2.0885 | −1.8455 | −0.6885 | −0.5765 |
| EN1 | 0.9405 | −1.6795 | 0.5715 | −0.0335 | −0.6155 | 0.4755 | −1.7835 | −0.5895 | −0.2385 | 0.3465 | 0.1785 | 0.8405 | −0.1485 | 0.8005 | 0.7405 |
| ENAH | −1.3745 | −0.8155 | −1.1925 | −0.2675 | 0.0805 | −1.0645 | −1.0465 | −1.6345 | 0.6315 | 0.7965 | −0.2805 | −0.6295 | −1.4275 | −0.6985 | 0.3045 |
| EPHX2 | −1.762 | 0.005 | −0.642 | −0.888 | −1.315 | −0.882 | −2.577 | −2.922 | −1.763 | −2.387 | −1.49 | −0.613 | −1.271 | −1.837 | −1.178 |
| EPLIN | −1.1655 | −0.3015 | −0.8315 | −0.2745 | −0.5015 | 0.8395 | 0.0735 | −0.1935 | −0.0115 | −0.0875 | 0.0445 | −0.2585 | −0.0315 | 0.0205 | 0.0585 |
| EPN3 | −0.4385 | 1.9585 | −0.7515 | −1.4115 | −0.5595 | −0.8285 | −2.5425 | −1.2446 | −0.5195 | −0.6515 | 0.2995 | −1.1985 | −1.2315 | −0.1785 | −1.2445 |
| EPOR | 0.693 | 0.2 | 1.723 | 0.131 | 0.105 | 0.648 | −0.328 | 0.458 | −0.33 | −0.823 | −0.126 | 0.099 | −1.473 | −0.267 | −0.804 |
| ERBB2 | −1.9855 | −2.7215 | 0.2665 | −2.9125 | −3.0325 | −5.0925 | −3.7125 | −2.2715 | −0.5695 | −1.4785 | −0.7085 | −1.2055 | −1.5865 | −0.1965 | −1.2855 |
| ERP70 | −0.255 | 0.598 | 0.159 | −0.158 | −0.03 | −0.222 | 0.239 | −0.843 | 0.158 | −0.371 | −0.018 | 0.367 | −0.308 | 0.154 | 0.004 |
| ESR1 | 1.7025 | 0.2045 | 0.2146 | 0.0005 | 0.3505 | 0.8295 | 0.3135 | 1.1715 | −0.2645 | 0.0475 | 0.0795 | 0.3415 | −0.0805 | 0.2815 | 0.7095 |
| ETFA | −0.8775 | −0.1775 | −0.1005 | −0.1425 | 0.4415 | 0.3755 | 0.2855 | 0.4135 | −0.0395 | −0.0885 | 0.4895 | −0.7225 | −0.7805 | −0.2825 | 0.3115 |
| ETV8 | 2.0965 | 1.3155 | 1.3745 | 0.7395 | 0.4065 | 0.9435 | 2.1585 | 1.0055 | −0.6985 | −1.1785 | −0.6165 | −0.2535 | −0.5335 | −0.1415 | −0.8135 |
| EVER1 | 0.5885 | −0.0035 | 0.2215 | 0.3985 | 0.7335 | −0.3455 | 0.7385 | −0.1545 | −0.1135 | 0.1155 | 0.2725 | −0.4505 | 0.1655 | 0.4375 | −0.1185 |
| EXO1 | −0.0205 | −0.1425 | −0.0695 | 0.2425 | 0.0855 | 0.7345 | 0.8855 | −0.1585 | −0.0105 | −0.0345 | 0.2265 | 0.8325 | −0.5245 | −0.4135 | −0.0475 |
| EXOSC9 | 1.01 | −0.298 | 1.355 | 1.318 | −0.14 | 0.305 | 2.277 | 2.235 | −1.346 | −1.197 | −0.064 | −0.754 | −0.868 | −0.9 | −1.469 |
| EXT2 | −0.079 | −1.828 | 0.14 | −0.155 | −0.701 | −1.264 | −1.484 | −1.16 | −1.821 | −1.806 | −0.81 | −0.053 | −1.849 | −0.632 | −1.181 |
| EZH2 | −0.3525 | −0.2665 | 0.3205 | 0.2265 | 0.5575 | 0.2505 | −0.2275 | −1.1295 | 0.1865 | −0.0815 | 0.0305 | −0.0955 | 0.1905 | 0.2236 | 0.4985 |
| FI1R | −3.163 | −0.099 | −0.829 | 0.632 | −0.863 | −1.792 | −1.215 | −0.02 | −1.168 | −2.127 | −1.551 | 1.384 | −0.607 | 0.565 | 0.83 |
| F9 | 1.461 | 2.128 | 6.986 | 2.694 | 2.28 | 1.542 | 1.831 | 1.428 | 0.126 | 2.554 | 4.909 | 2.739 | 0.932 | 2.004 | 1.785 |
| FA2H | 0.115 | 0.37 | −0.353 | −0.15 | 0.007 | −0.223 | −0.438 | 0.027 | −0.708 | −0.304 | 1.147 | 0.337 | −0.04 | −0.522 | −0.536 |
| FABP7 | −0.788 | −0.342 | 0.423 | 0.13 | −0.209 | −0.299 | −1.349 | −1.586 | −0.998 | −1.595 | −0.77 | 0.796 | −1.53 | −1.443 | −0.864 |
| FADD | 0.111 | −0.214 | −0.602 | −0.371 | −0.101 | 0.113 | 0.166 | 0.138 | 0.134 | −0.052 | 0.396 | 0.626 | −0.002 | 0.858 | 0.724 |
| FAHD1 | 0.373 | −0.92 | −0.381 | 0.421 | 0.872 | 0.853 | 1.826 | −1.088 | −0.612 | −1.069 | −0.654 | −1.116 | −0.241 | −0.668 | −1.138 |
| FAHD2A | 0.34 | −0.252 | −0.373 | −0.311 | 0.166 | 0.069 | 0.232 | −0.134 | −0.016 | 0.116 | 0.116 | 0.226 | 0.151 | −0.248 | 0.247 |
| FAM38A | −1.1705 | −0.1455 | −0.1015 | 0.7015 | −0.3145 | −1.6025 | 0.9185 | 0.3195 | 0.0875 | 0.2885 | 0.1755 | 3.1305 | 0.8965 | 2.5415 | 1.7495 |
| FAM3A | 1.4355 | 0.4985 | 1.3795 | 0.0045 | 1.2255 | 0.0475 | 1.3025 | −0.4365 | −0.2676 | −0.0505 | −0.3755 | −0.7035 | −1.5155 | −0.9985 | −0.6415 |
| FAM3B | −0.771 | 2.007 | 1 | −0.128 | 0.198 | −0.146 | −0.401 | 2.376 | 1.03 | 0.87 | 0.31 | −0.005 | −0.198 | 0.38 | −0.188 |
| FAM3C | 0.828 | 1.085 | 2.284 | 1.004 | 1.378 | 0.118 | 0.533 | −0.401 | −0.758 | −0.598 | −0.334 | −0.796 | −2.372 | −1.379 | −1.014 |
| FAM46C | 2.337 | 1.347 | 1.705 | 1.742 | 0.48 | 0.432 | 3.059 | 0.875 | −0.972 | −1.498 | −0.541 | −0.21 | −2.187 | −0.925 | −1.437 |
| FAM49B | 0.517 | 1.117 | 2.37 | 1.145 | −0.443 | 0.614 | 2.177 | 2.428 | −1.068 | −0.983 | 0.645 | 0.127 | −0.253 | −0.648 | −0.603 |
| FAM54A | 1.11 | 1.302 | 1.504 | 1.428 | 0.714 | 0.616 | 0.876 | 0.836 | 0.111 | −0.009 | 0.353 | 0.559 | 0.672 | 0.399 | 0.295 |
| FANCA | 0.9165 | −0.0715 | 0.4955 | 0.3705 | 0.1805 | −0.0005 | −0.0795 | 1.5035 | 0.1365 | 0.4635 | 0.4955 | 0.7016 | 0.3615 | 1.3255 | 0.6055 |
| FANCE | −2.7525 | −2.4105 | −0.9865 | −1.6865 | −0.7855 | −2.2675 | −3.0155 | −1.9375 | −0.8495 | −2.1345 | 0.1985 | −1.0095 | −1.2495 | −0.8605 | −1.8815 |
| FBL | 0.349 | −0.119 | −0.474 | −0.326 | −0.28 | 0.385 | −1.051 | 0.408 | 0.617 | 0.956 | 0.515 | −0.621 | −0.407 | 0.192 | −0.358 |
| FBP1 | −0.112 | 0.901 | 0.267 | −0.016 | 0.351 | 0.25 | −0.236 | −0.162 | −0.342 | −0.492 | −0.509 | 0.54 | 0.079 | 0.125 | −0.116 |
| FBS1 | 1.8865 | −0.9525 | 0.7416 | −0.1015 | 0.3515 | 1.3345 | −0.8905 | −0.0045 | 0.1975 | −0.0515 | 0.1195 | 0.2285 | −0.6675 | −0.1075 | −0.3615 |
| FBXL20 | −1.063 | 0.1 | −0.888 | −0.291 | −0.464 | −0.296 | 0.116 | −0.157 | 0.249 | −0.518 | 0.084 | −0.144 | −0.373 | −0.103 | −0.397 |
| FBXO25 | 0.885 | 0.105 | 0.485 | 0.855 | 1.217 | 0.018 | 0.558 | −0.42 | −0.337 | −0.089 | −0.101 | 0.114 | 0.194 | 0.386 | 0.064 |
| FDXR | −0.537 | −0.062 | −0.823 | 0.561 | 0.162 | 0.202 | −1.101 | 0.245 | −0.486 | −0.543 | −1.056 | −0.175 | −0.186 | −0.32 | 0.542 |
| FEM1B | −1.549 | 0.429 | −2.266 | 0.031 | 0.198 | −2.69 | −0.932 | −3.119 | −0.224 | 0.169 | −0.292 | −0.108 | −0.754 | −0.805 | 0.166 |
| FGF12 | −1.7455 | 0.2055 | −0.9485 | −0.2925 | −0.3155 | −0.4075 | −0.2615 | −0.3485 | 0.1526 | 0.0135 | −0.0855 | 0.0005 | 0.3645 | −0.2225 | 0.2855 |
| FGF13 | 0.038 | 2.282 | 1.696 | 0.605 | 0.669 | 0.251 | −0.173 | 1.584 | 0.205 | −0.656 | −0.01 | 0.957 | −0.242 | 0.39 | 0.252 |
| FHOD1 | 2.3135 | −0.0535 | −0.1075 | −0.0515 | 0.2145 | −0.3995 | −0.4325 | 0.7055 | −0.0825 | 0.0805 | −0.4245 | −0.4955 | 0.1495 | −0.3155 | −0.0705 |
| FKBP11 | −0.3235 | −0.5405 | 0.3615 | −0.6215 | −0.6395 | −0.8555 | −1.3825 | −1.4615 | −1.3515 | −1.8585 | −0.8005 | 0.4695 | −2.1335 | −0.6575 | −1.0035 |
| FLJ10094 | 0.023 | −0.396 | 0.207 | 0.498 | 0.227 | −0.359 | 0.83 | 0.354 | 0.642 | 0.741 | −0.042 | 1.512 | 1.043 | 0.604 | 0.995 |
| FLJ10115 | 0.2015 | 0.0705 | 0.9775 | 0.6835 | 0.6615 | 0.3285 | 0.0225 | −0.2205 | −0.3825 | −0.8775 | 0.1855 | 0.1875 | −0.8025 | −0.2085 | −0.3105 |
| FLJ10159 | −0.275 | −0.491 | 0.088 | −0.208 | −0.426 | −0.256 | −0.646 | 0.126 | 0.061 | 0.423 | 0.216 | −0.215 | −0.646 | −0.313 | −0.251 |
| FLJ10204 | 2.608 | 0.85 | 0.599 | −0.158 | 1.141 | 0.851 | 1.695 | 0.302 | −1.123 | −1.062 | −0.723 | −0.417 | −1.148 | −1.252 | −1.04 |
| FLJ10275 | −1.6375 | −1.7535 | −2.2415 | −0.0455 | 0.7205 | −1.6465 | −0.6745 | −1.8945 | 1.5725 | 2.6185 | 1.2895 | 1.2295 | 0.9805 | −0.0855 | 0.4835 |
| FLJ10292 | 0.1485 | −0.2585 | 0.0285 | 0.0805 | 0.1815 | 0.1775 | −0.9405 | 0.1425 | −0.5595 | −0.3445 | −0.7015 | 0.1315 | −0.3045 | −0.1035 | −0.1605 |
| FLJ10324 | 1.8495 | 0.4895 | 1.7415 | 0.5355 | 0.6475 | −0.6246 | 0.6255 | 1.1945 | −1.1075 | −0.7455 | 0.2685 | 0.1145 | −0.3825 | −0.5515 | −0.8795 |
| FLJ10700 | 0.822 | −0.549 | 0.178 | 0.243 | −0.081 | 0.487 | 0.402 | 0.444 | 0.257 | 0.028 | 0.037 | 0.38 | −0.249 | −0.517 | −0.463 |
| FLJ10706 | 1.581 | 1.013 | 0.377 | 0.274 | 2.032 | 1.348 | 1.274 | 2.294 | −0.704 | −0.342 | 0.865 | −0.543 | −1.076 | 0.125 | −0.643 |
| FLJ10671 | −1.203 | −0.318 | −1.768 | −1.14 | −0.165 | 1.202 | −1.351 | −0.544 | 0.167 | 0.361 | −0.001 | 0.105 | 0.708 | 0.846 | −0.378 |
| FLJ10901 | −2.0165 | −0.5185 | −0.1195 | −0.5505 | −1.0635 | −1.2445 | −1.4095 | −0.9795 | −0.7025 | −0.8305 | −0.0395 | −2.2505 | −0.6245 | −0.0375 | −0.9205 |
| FLJ10918 | −0.415 | 1.886 | −0.403 | 0.139 | 0.884 | −0.015 | 0.42 | 0.437 | 0.315 | 0.162 | 0.110 | 1.296 | −0.505 | 0.587 | 0.905 |
| FLJ10980 | 1.8875 | 0.1265 | 0.6195 | 0.1805 | −0.3565 | 0.2245 | −0.3745 | −0.4495 | −0.1245 | 0.1735 | −0.4615 | −1.1135 | −0.2435 | −0.2555 | 0.0285 |
| FLJ11017 | 0.7095 | 1.6375 | 0.7215 | 1.0205 | 1.1345 | −0.4076 | 0.8495 | −0.0295 | −0.4715 | −0.7035 | −0.0445 | −0.3455 | −1.6065 | −1.1325 | −0.8235 |
| FLJ11088 | −0.5545 | 0.2755 | −0.4775 | −0.1335 | −0.1445 | 0.5675 | 0.1795 | 0.2985 | −0.6535 | 0.1165 | −0.1745 | −0.3685 | 0.2225 | 0.0565 | 0.2725 |
| FLJ11151 | −0.5115 | −1.0295 | −0.6415 | −0.6565 | −0.0335 | −0.1985 | −1.3305 | −1.0595 | −0.0705 | −0.3265 | −0.0315 | −0.4805 | −0.0595 | −0.1355 | −0.1945 |
| FLJ11267 | −0.1085 | 0.0116 | −0.5285 | −0.0245 | −0.4845 | −0.1345 | −0.1455 | −0.6505 | −0.3495 | −0.6065 | 0.0255 | −0.2035 | 0.0375 | −0.7175 | −0.6075 |
| FLJ11280 | −0.5415 | 0.1165 | 0.4755 | −0.4395 | 0.1005 | 0.4755 | 0.5225 | 0.2815 | 0.1245 | 0.4945 | 0.4385 | 0.1545 | −0.4965 | 0.6285 | 0.8335 |
| FLJ11508 | 1.7605 | −0.3495 | 0.1925 | 0.3235 | 0.6075 | −0.7015 | 0.2095 | 0.1605 | −0.5885 | 0.1095 | −0.5175 | 1.2175 | 0.0545 | −0.1285 | −0.8045 |
| FLJ12270 | −2.2695 | −1.2365 | −1.3515 | −0.6965 | −1.8615 | −0.7095 | −0.1955 | −0.3455 | −0.5585 | −0.6555 | −1.1085 | −0.1446 | −0.0855 | −0.2855 | −1.2325 |
| FLJ12844 | 1.5635 | 0.4825 | −0.5876 | −0.2685 | −0.1215 | 0.7385 | −0.0765 | 0.6015 | 0.3685 | −0.0755 | 0.5815 | −0.0335 | 0.1255 | −0.2145 | −0.1685 |
| FLJ12650 | 1.0175 | 0.1675 | 0.8015 | 0.4045 | 0.0985 | −0.3015 | 1.3935 | 0.2055 | −0.8345 | −0.7105 | −0.3755 | 3.0835 | −0.2845 | 0.2135 | −0.1385 |
| FLJ12684 | 0.8715 | 0.4675 | −0.2095 | −0.1585 | −0.2845 | 0.8995 | 0.4805 | 0.4785 | 0.2245 | −0.0555 | 0.0065 | 0.3305 | −0.7445 | 0.6375 | 0.6405 |
| FLJ12735 | −0.636 | −1.677 | −0.267 | −0.283 | −1.496 | 0.602 | −0.412 | −0.72 | 0.458 | 0.982 | −0.296 | 0.534 | −0.028 | 0.539 | 0.63 |
| FLJ12750 | −3.398 | −3.286 | −1.711 | −1.009 | −2.386 | −3.703 | −1.873 | −2.591 | −1.074 | −0.925 | −1.175 | 0.755 | −0.682 | 0.781 | −0.985 |
| FLJ12895 | 0.3485 | −0.0625 | 1.0995 | 0.1675 | 0.7475 | −0.0355 | −0.1345 | −0.4115 | −0.0225 | 0.1235 | −0.0375 | −0.2845 | −1.0875 | 0.0175 | −0.5475 |
| FLJ13710 | 1.2935 | 1.5435 | 2.0005 | 0.9865 | −0.0375 | −0.2365 | 0.2875 | 0.4485 | −1.4235 | −0.6865 | −0.7485 | 0.9125 | −0.3625 | −0.1165 | 0.0095 |
| FLJ13855 | −0.32 | −0.259 | 0.4 | 0.217 | −0.537 | 0.724 | 0.666 | 1.073 | −0.349 | 0.338 | 0.297 | −0.355 | 0.537 | 0.837 | 0.397 |
| FLJ13912 | 1.206 | −0.449 | 1.496 | −0.403 | 0.273 | 0.383 | 1.122 | 1.426 | −1.129 | −1.161 | −0.406 | −0.392 | −0.976 | −0.824 | −0.517 |
| FLJ14124 | 1.279 | 0.602 | 0.3 | 0.085 | 0.592 | 0.884 | 0.252 | 0.433 | −0.917 | −0.747 | −0.202 | 0.068 | 0.048 | 0.052 | −0.123 |
| FLJ14627 | 0.0175 | −0.5805 | −0.2495 | 0.0205 | −0.2895 | −0.4335 | 3.3175 | −0.1385 | 0.3115 | 0.1005 | −0.0405 | 0.4285 | 0.3455 | −0.1615 | −0.1115 |
| FLJ14666 | −0.028 | 1.458 | 1.512 | 0.381 | 1.797 | 1.348 | 1.715 | 0.392 | −0.958 | −0.376 | −1.201 | −1.55 | −2.001 | −0.91 | −0.509 |
| FLJ14904 | −1.938 | −2.55 | −2.433 | −0.799 | 0.16 | −0.74 | −0.724 | −0.91 | 0.36 | −0.209 | −0.015 | −0.814 | −0.578 | −1.042 | −0.37 |
| FLJ20035 | 0.001 | 0.566 | −0.229 | −0.131 | 1.04 | 0.818 | 0.446 | −0.513 | −1.075 | −0.939 | −0.314 | −0.2 | −0.082 | −0.397 | −1.272 |
| FLJ20152 | 1.268 | 0.094 | −0.28 | 0.19 | −0.35 | −0.375 | −0.464 | 0.265 | 0.134 | 0.077 | 0.604 | −0.151 | 0.104 | 0.243 | 0.019 |
| FLJ20171 | −1.793 | −0.273 | −0.427 | −0.799 | −0.201 | −0.289 | −1.357 | −1.418 | −1.146 | −1.833 | −0.015 | −1.282 | −2.534 | −0.169 | −0.789 |
| FLJ20244 | −0.833 | 2.106 | −0.56 | −0.441 | −0.712 | −0.379 | −0.486 | 0.272 | −0.898 | −0.789 | −0.573 | 0.378 | −1.309 | 0.598 | −0.77 |
| FLJ20273 | −0.526 | −0.298 | 0.114 | 0.03 | 0.685 | −0.147 | 0.238 | −0.547 | −0.177 | 1.255 | 1.324 | −0.15 | 0.142 | 0.294 | 0.128 |
| FLJ20315 | 0.894 | 0.856 | 1.089 | 1.565 | 0.739 | −0.334 | 1.503 | −0.511 | −0.362 | −0.223 | 0.037 | 0.231 | 1.023 | −0.028 | −0.173 |
| FLJ20323 | 0.3175 | 0.4596 | −0.2925 | 0.2895 | 0.4585 | 1.2955 | 0.2325 | −0.2665 | −1.2915 | −1.0975 | −0.7855 | −0.8815 | −1.6515 | −0.9605 | −0.8795 |
| FLJ20518 | 0.48 | 0.997 | 1.601 | 0.588 | 1.148 | 0.235 | 0.291 | 0.169 | −0.463 | 0.14 | 0.142 | −2.432 | −0.460 | −0.46 | −0.213 |
| FLJ20530 | 0.328 | 0.395 | 0.676 | 0.386 | −0.139 | −0.296 | −0.563 | −0.741 | −0.307 | −0.753 | −0.216 | 0.285 | −0.71 | −0.037 | −0.455 |
| FLJ20696 | 0.7705 | −0.2035 | 1.3205 | −0.0775 | 0.4745 | 0.0605 | 0.1715 | −0.8455 | −1.2285 | −1.1945 | −0.7975 | 0.2835 | −0.0545 | −0.1495 | −0.1015 |
| FLJ20718 | 0.235 | 0.048 | 0.702 | −0.335 | 0.507 | 0.084 | 0.309 | −0.051 | −0.185 | −0.78 | −0.75 | −0.205 | −0.485 | 0.5 | −0.201 |
| FLJ20772 | −1.3455 | −0.3045 | −2.0265 | −1.1035 | −0.1195 | −0.1305 | −0.8355 | −1.6905 | 0.1545 | 0.2055 | 0.4425 | 0.0185 | −1.1405 | 0.2975 | −0.0925 |
| FLJ20989 | −0.3355 | −0.2545 | −1.2325 | −0.1875 | −0.6955 | 0.1485 | −0.7185 | −0.7445 | 0.6245 | 1.2915 | 0.4815 | −1.3285 | −0.1835 | 0.1365 | 0.2075 |
| FLJ21019 | 0.2375 | −0.4905 | −1.1655 | −0.6785 | −0.2375 | 0.2945 | −0.0655 | −0.0435 | −0.7385 | −0.8965 | −0.7105 | −1.8625 | −1.2055 | −0.4255 | −0.4035 |
| FLJ21062 | 0.5005 | −0.7785 | 0.7055 | −0.0715 | −1.6565 | 1.1135 | 0.0075 | −2.4555 | −0.0925 | 0.3085 | 0.0175 | 0.1575 | 0.3705 | 0.9635 | 0.8775 |
| FLJ21159 | −0.2095 | −1.6995 | −1.0345 | −0.3195 | −0.2145 | 1.2185 | −1.0065 | −0.7305 | −0.0885 | −0.1475 | −0.1095 | 0.1575 | −0.2715 | 0.3915 | −0.2215 |
| FLJ21616 | −1.825 | −0.29 | −1.067 | −0.678 | −0.581 | −0.81 | −1.805 | −0.957 | 0.217 | −0.279 | −0.547 | −1.415 | −1.039 | −0.28 | 0.09 |
| FLJ21827 | −0.6255 | −0.4235 | −0.1755 | −0.7545 | 0.0095 | 0.4555 | 0.1665 | −0.9475 | −0.7125 | −0.6905 | −1.3685 | −0.5625 | −1.9555 | −0.2715 | 0.1935 |
| FLJ21963 | −0.77 | 0.113 | 0 | −0.032 | 0.051 | 0.518 | 1.343 | 0.146 | 0.128 | −0.132 | 0.071 | 0.289 | −0.535 | 0.345 | −0.153 |
| FLJ22104 | 0.0275 | 0.8535 | 1.5965 | 1.0615 | 1.5785 | −0.0175 | −0.4155 | 0.9545 | −0.6605 | 0.0806 | 1.0855 | −0.2235 | −0.4225 | −0.1725 | −0.1555 |
| FLJ22222 | −0.909 | −0.747 | 0.893 | 0.319 | −0.731 | 0.254 | 0.983 | 0.042 | −0.378 | 0.268 | −0.094 | −1.351 | 0.393 | 0.378 | −0.149 |
| FLJ22573 | 0.9575 | −0.1365 | 0.8855 | −0.6085 | −0.8575 | −1.0825 | −0.9525 | −0.1595 | −0.4575 | −0.9705 | −0.5145 | −0.4275 | −0.6565 | −1.1255 | −0.6995 |
| FLJ22794 | 0.653 | 0.095 | −0.019 | −0.29 | 0.774 | 1.338 | 0.774 | 0.419 | −0.189 | −0.189 | −0.89 | 0.748 | −1.391 | −0.538 | −0.252 |
| FLJ23188 | 2.2335 | 0.5805 | −0.1375 | 0.2755 | 0.8825 | −0.2185 | 0.0285 | 0.3995 | −0.5535 | −0.2685 | −0.4275 | −0.1985 | 0.2515 | −0.1975 | −0.5545 |
| FLJ23441 | 0.175 | −1.584 | 1.024 | −0.222 | −0.499 | −0.393 | −0.057 | −0.596 | −1.58 | −1.406 | −0.174 | −0.539 | −0.722 | −0.516 | −1.35 |
| FLJ25471 | 0.093 | 0.282 | 0.184 | 0.223 | 0.73 | 0.485 | −0.43 | −0.402 | −0.432 | −0.327 | 0.066 | −0.838 | −1.51 | −0.112 | −0.598 |
| FLJ31204 | −1.5335 | 0.3525 | −0.1205 | 0.3035 | −0.5275 | 2.4765 | −0.0035 | 0.7495 | 0.8215 | 0.0355 | 0.3775 | 0.5245 | 1.1585 | 0.6495 | 1.0215 |
| FLJ31795 | −1.0925 | −0.1115 | 0.9055 | 0.9715 | 1.1045 | 0.2435 | −0.0785 | 0.9875 | −0.0815 | 0.6095 | 0.6515 | 0.5515 | 0.8555 | 0.1005 | 0.5545 |
| FLJ32942 | −1.7816 | −1.3475 | −2.2565 | −1.0805 | −0.6615 | −1.5175 | −1.1828 | −2.0595 | 0.7185 | −0.6735 | −1.1255 | −0.6605 | −0.3725 | −1.3285 | −0.1835 |
| FLJ37970 | −2.711 | −0.37 | −0.087 | 0.889 | 2.575 | 2.68 | 2.064 | −0.554 | 0.204 | −0.068 | 1.565 | 1.03 | 1.153 | 0.464 | 0.432 |
| FLJ39370 | −0.8975 | 0.5185 | −0.1795 | −0.1805 | 0.1015 | 0.6815 | 0.8665 | 0.6565 | 0.1495 | 0.4655 | 0.3835 | 0.2775 | −0.6965 | 0.2925 | 0.2865 |
| FLJ90588 | −1.6865 | 0.8065 | −0.6395 | 0.5835 | 0.1335 | −1.3385 | −0.2835 | 1.0315 | 1.1875 | 0.4585 | 1.0055 | 1.8965 | 1.9325 | 1.4015 | 2.1455 |
| FN3KRP | −5.996 | −5.086 | −4.957 | −4.486 | −5.646 | −7.89 | −6.316 | −5.078 | −2.809 | −3.84 | −1.091 | −2.586 | −2.546 | −0.861 | −4.876 |
| FOLR1 | 3.1235 | 1.4475 | −0.3095 | 3.9105 | 1.2785 | 2.5805 | 2.6705 | 3.5905 | 2.0705 | 1.8405 | 0.9605 | 1.9265 | 1.2995 | 2.5005 | 2.4135 |
| FOXA1 | −0.9745 | −1.1985 | −1.1325 | −0.1285 | −0.0815 | −1.2775 | −0.5335 | −0.8995 | 0.1285 | 0.7275 | −0.0125 | −0.8395 | 1.4395 | 0.6325 | 0.0205 |
| FOXC1 | 0.819 | 1.227 | 0.345 | 0.248 | 0.719 | 0.633 | 1.179 | −0.25 | −0.448 | −1.341 | −0.473 | 0.002 | −1.202 | −1.304 | −0.999 |
| FOXP1 | −0.5525 | −0.2495 | −0.3215 | 0.0465 | −0.4875 | −0.0075 | −0.5345 | −0.4815 | 0.7635 | 0.3655 | 0.0065 | 1.1035 | 0.4425 | 1.0815 | 0.4965 |
| FREQ | 0.4715 | 0.0605 | 0.4415 | 0.4865 | 0.1575 | −0.1855 | 0.7305 | 0.0275 | −0.0595 | −0.1515 | −0.0485 | 0.3425 | −1.9075 | −0.3985 | −0.7455 |
| FTO | −1.2925 | −0.3565 | 1.1745 | −0.1395 | 0.4835 | −0.5885 | 0.0075 | 0.6535 | 0.1825 | 0.1575 | 1.2335 | −0.0545 | −1.8185 | −1.1505 | −0.2515 |
| FUBP1 | −0.7785 | −0.4525 | −0.2495 | −0.5176 | −0.2345 | −1.6345 | −1.4085 | 0.1025 | −0.5755 | −1.1395 | −1.0575 | −0.2205 | −0.3965 | −1.2115 | −1.4495 |
| FUCA1 | 0.834 | −0.273 | −0.845 | 0.254 | 1.195 | 1.377 | 0.871 | 0.981 | 1.265 | 1.571 | 0.857 | 0.436 | 1.267 | 1.013 | 0.796 |
| FUT8 | −0.4875 | −1.0685 | −0.2715 | −0.3655 | −0.2055 | −0.4425 | −0.7165 | −1.7085 | 0.0745 | 0.0525 | −0.3785 | −0.1985 | −1.1745 | −0.5485 | 0.2275 |
| FXYD6 | 1.237 | 1.143 | −0.073 | 1.01 | −0.229 | −1.071 | −0.834 | 1.067 | 0.241 | 0.678 | 0.664 | 1.432 | 0.944 | 1.696 | 0.488 |
| FYCO1 | 1.156 | 0.869 | 1.835 | 1.182 | 0.52 | −0.315 | −0.247 | 2.21 | 0.581 | 1.913 | 0.894 | 2.493 | −0.094 | 0.756 | 0.868 |
| FZD10 | 1.2385 | 0.4375 | −0.3785 | 0.2405 | 1.5565 | 0.8485 | 1.0835 | 1.0325 | 0.2815 | 0.1615 | 0.3465 | 1.3055 | 0.2725 | 0.4935 | 1.0875 |
| FZD7 | 9.4495 | 1.4955 | 0.7085 | 0.3155 | 2.8385 | 2.0975 | 2.4155 | 0.1175 | −0.8485 | −1.6615 | −1.1475 | −1.3925 | −0.9245 | −1.4165 | −1.4965 |
| FZD9 | −0.5155 | 1.7065 | 0.2995 | −0.1565 | 0.7945 | 1.6225 | 2.1485 | 0.3565 | 0.1385 | −0.1225 | 0.3865 | −0.7535 | −0.0985 | −0.2715 | −1.0035 |
| G1P2 | 1.6835 | −0.1695 | 0.2785 | 0.4105 | 0.2655 | −0.3115 | 1.0545 | 0.4045 | −0.1715 | 0.0185 | −0.1935 | 0.3455 | −0.0455 | 0.4815 | 0.1125 |
| G1P3 | −0.734 | −0.205 | 1.384 | 0.042 | 0.806 | −0.073 | 0.459 | −1.434 | −0.198 | −0.088 | −0.628 | 0.592 | −0.63 | −0.258 | 0.063 |
| GA17 | 1.5605 | 1.0455 | 1.3785 | 0.8925 | 0.8615 | 1.2545 | 2.4505 | 0.3275 | −1.5805 | −1.1875 | −0.1805 | 0.0015 | −0.6295 | −0.3395 | −1.4635 |
| GABARAP | −9.8835 | 0.0085 | −1.6375 | −0.7605 | −0.7545 | 0.1025 | −0.3085 | −0.7895 | −0.5495 | −1.0245 | 0.1265 | −0.4295 | −0.5745 | −1.1245 | −0.7885 |
| GAJ | 2.9885 | −0.5705 | 3.8175 | 0.8505 | −0.4105 | −1.0285 | −1.6385 | 0.8395 | −0.1495 | −0.1235 | 0.9335 | 0.8195 | 0.5615 | 0.1395 | −0.0985 |
| GALE | −0.005 | 0.346 | 0.259 | −0.177 | 0.604 | 0.663 | 0.284 | −0.102 | 0.046 | −0.635 | −0.155 | −0.434 | −0.658 | −0.416 | −0.308 |
| GALNT14 | −1.0025 | −1.4155 | −0.2415 | −0.9485 | −1.6515 | −1.7875 | −1.7975 | −1.6425 | −1.3135 | −1.8995 | −1.4795 | −1.4575 | −1.3595 | −1.0525 | −1.1495 |
| GALNT2 | −3.3365 | −1.4515 | −1.9255 | −0.9775 | −0.9005 | −1.3645 | −0.9795 | −1.3705 | −0.1976 | −0.3915 | 1.0125 | 0.5355 | 0.2305 | 0.8116 | 0.6275 |
| GALNT7 | 0.558 | 0.987 | −0.385 | 0.027 | 0.418 | 1.564 | 0.659 | 1.786 | 0.211 | 0.307 | 0.59 | 0.096 | −1.222 | 0.271 | −0.127 |
| GAMT | 0.268 | 0.121 | 0.128 | −0.202 | 0.387 | 0.277 | 0.142 | 0.557 | −0.166 | −0.398 | 0.048 | −0.756 | −0.244 | −0.193 | −0.876 |
| GAPD | 1.102 | 1.295 | 0.621 | 0.389 | 0.444 | 1.034 | 0.566 | 0.255 | −0.377 | −0.409 | −0.14 | −0.022 | −1.105 | −0.168 | −0.835 |
| GARS | −3.32 | −3.33 | −2.475 | −2.889 | −2.418 | −2.002 | −2.997 | −1.577 | −1.389 | −1.283 | 0.019 | −1.822 | −0.237 | −0.684 | −1.788 |
| GART | −1.2355 | −0.0195 | −0.2915 | 0.4475 | −0.2995 | −0.7445 | −1.1485 | −0.0605 | −0.4455 | 0.2035 | 0.4375 | 0.3605 | −1.2435 | −0.6285 | −0.8195 |
| GATA3 | 0.5045 | 0.2965 | −0.0035 | −0.5215 | 0.2495 | 0.4805 | −0.2805 | 0.0035 | −0.6265 | −0.7785 | −0.2775 | −0.6915 | −0.7945 | −0.8155 | −0.7915 |
| GATM | 0.198 | 0.355 | 0.239 | 0.159 | 0.53 | −0.565 | −0.83 | −0.179 | −1.286 | −0.13 | 0.138 | −0.83 | −0.648 | −0.018 | −0.524 |
| GBA | 0.865 | 0.819 | 0.447 | −0.168 | 1.59 | 0.862 | −0.046 | −0.488 | −0.211 | 0.149 | −0.161 | 0.257 | −1.831 | 0.354 | 0.137 |
| GDAP1 | −0.528 | 0.552 | −0.626 | −0.192 | −0.531 | −0.381 | −0.98 | −0.284 | 0.28 | 0.285 | 0.72 | −0.528 | 0.139 | 0.183 | −0.399 |
| GDI2 | −0.5405 | −0.5685 | −0.8255 | −0.5115 | −0.5855 | −0.0495 | 0.3715 | −0.3445 | −0.2745 | −0.2025 | −0.2845 | −1.4125 | −0.5355 | −0.1705 | −0.3065 |
| GGA1 | −0.9515 | 0.6345 | 0.2775 | −0.1325 | −0.4885 | −0.8555 | −0.5375 | 1.4295 | −0.0825 | −0.2725 | 0.2205 | −0.9605 | −0.1895 | −0.7925 | −0.8115 |
| GKAP1 | −0.9485 | 0.2915 | 0.9165 | −0.1285 | 0.4225 | 0.5595 | 0.9355 | 0.0215 | 0.2155 | 0.5255 | 0.0495 | 0.0005 | −0.3175 | 0.3105 | 0.2235 |
| GLCCI1 | −0.6385 | −0.6605 | −0.3845 | 0.1005 | −0.2315 | −0.7965 | −1.3495 | 0.9455 | 0.7735 | 1.1325 | 0.9925 | 1.0555 | 0.6685 | 1.2665 | 1.3545 |
| GLG1 | 0.833 | 2.637 | 1.206 | 0.682 | 1.355 | 0.795 | 0.811 | 0.106 | −0.851 | −0.276 | −0.548 | 0.123 | −0.627 | −0.521 | −0.341 |
| GLTSCR2 | −0.761 | −0.252 | −0.033 | −0.614 | −0.784 | 0.579 | −0.682 | −1.176 | −0.839 | −0.73 | −0.584 | −0.972 | −0.508 | −0.418 | −1.09 |
| GNB4 | 0.261 | −0.417 | −0.065 | 0.307 | −0.407 | −0.119 | 1.861 | 0.379 | −0.451 | −0.374 | −0.433 | −0.343 | 0.067 | −0.031 | −0.28 |
| GOLPH2 | 0.5915 | 0.6475 | −0.0765 | 0.2195 | 0.0205 | 0.2725 | 1.9345 | 1.2315 | 0.1735 | −0.0975 | 0.3385 | 0.3525 | 1.0895 | 0.4485 | 0.1865 |
| GPATC1 | 0.4425 | 0.4515 | −0.9305 | 0.5085 | 0.8785 | 0.8525 | 1.2915 | −0.3355 | 0.1355 | 0.4305 | 0.4225 | 0.5965 | 0.1335 | 0.7915 | 0.8985 |
| GPC2 | −1.9775 | −1.5665 | −1.3815 | −0.8215 | −0.8735 | −2.2865 | −1.2475 | −2.2095 | −1.1275 | −1.5455 | −1.3495 | −1.1235 | −0.7225 | −0.9565 | −1.5315 |
| GPR125 | −1.044 | 0.018 | −0.013 | 0.25 | 0.481 | 1.101 | −0.62 | −0.922 | 0.054 | −0.687 | −0.469 | 0.099 | 0.425 | −0.041 | −0.02 |
| GPR160 | −3.0905 | −1.0995 | −2.6485 | −0.8315 | −2.1545 | −1.2335 | −0.8385 | −1.0735 | −0.7065 | −1.3575 | −0.8225 | 0.2635 | −0.0445 | 0.0715 | −0.3235 |
| GPR56 | −2.5005 | −0.5365 | −2.2325 | −0.1085 | −0.3875 | 0.4885 | 0.4525 | −1.0835 | 0.1155 | 0.0815 | 0.4475 | 0.5675 | 0.7315 | 0.7235 | 0.3875 |
| GPRC5C | 2.1925 | 1.5465 | 1.5635 | 2.2475 | 2.1825 | 1.8705 | 1.4315 | 0.8825 | −1.1355 | −0.9145 | −0.2735 | −0.9395 | −1.7345 | −0.3285 | −1.2905 |
| GPSM1 | −2.262 | 0.017 | −1.02 | −0.633 | −2.08 | −1.163 | 0.786 | 0.72 | −0.143 | 0.721 | 0.452 | 1.307 | 0.331 | 0.457 | 0.578 |
| GPSM2 | −0.005 | 1.132 | −0.085 | 0.602 | −0.239 | 1.873 | 0.567 | 0.432 | 0.226 | −0.514 | −0.752 | 0.617 | −1.272 | −0.473 | 0.243 |
| GPT | 2.1855 | 0.2835 | 0.6185 | 0.2845 | 1.0635 | 1.0945 | 0.3725 | 1.6255 | −0.4535 | −0.1585 | −0.8805 | 1.7555 | −0.9135 | 0.1175 | −0.1175 |
| GPT2 | 1.874 | 1.228 | −0.952 | −0.068 | 1.329 | 1.027 | 2.811 | 1.898 | −0.474 | −0.977 | −0.953 | −1.009 | −0.334 | −0.512 | 0.23 |
| GPX7 | −0.093 | 1.2 | −0.279 | −0.127 | −0.143 | −0.208 | −0.336 | −0.132 | 0.853 | 0.635 | 0.748 | −0.329 | 1.217 | 0.188 | −0.027 |
| GRB14 | −1.7235 | 0.5035 | −1.4785 | −0.5055 | 0.0165 | −0.8045 | −0.4035 | 0.1565 | −0.0205 | 1.3735 | 0.9505 | 0.4165 | −0.2155 | −0.5055 | 1.1155 |
| GRB7 | −0.8105 | 0.7545 | −0.0635 | −0.2595 | −0.5385 | 0.0955 | −0.2635 | 0.1715 | −0.3275 | 0.0275 | 0.0415 | −0.0945 | 0.4775 | 0.3855 | 0.3835 |
| GSC | −0.33 | −0.337 | −0.468 | −0.138 | 0.022 | 0.328 | 0.41 | 0.038 | −0.888 | −0.344 | −0.822 | −0.715 | −0.849 | −0.075 | 0.011 |
| GSDML | 0.359 | 0.406 | 0.242 | 3.098 | 1.87 | 0.937 | 0.641 | 0.262 | −0.02 | 0.796 | 0.219 | 0.388 | 0.364 | 0.427 | 0.333 |
| GSK3B | −0.685 | −0.573 | 0.045 | −0.275 | −0.137 | −0.018 | −0.753 | −0.115 | 0.558 | 0.203 | 0.591 | 0.36 | 0.348 | 0.111 | 0.25 |
| GSTA1 | −2.0985 | −0.2885 | −3.5065 | −1.3825 | −0.9885 | −2.4725 | −1.3205 | −1.7705 | −0.6265 | −0.3925 | 0.1395 | −0.0815 | −0.3115 | −0.3365 | −0.4885 |
| GSTK1 | 1.68 | 0.663 | 1.082 | 0.142 | 1.438 | 1.048 | 0.885 | 1.755 | 0.683 | 0.345 | 0.546 | 0.328 | −0.11 | 0.412 | 0.176 |
| GSTM3 | −0.3395 | −0.2485 | 0.0125 | −0.8005 | 0.1405 | 0.4925 | −0.8105 | −0.4325 | −0.1995 | −1.9835 | 0.4095 | 0.1425 | 0.0705 | 0.0445 | −1.2715 |
| GSTP1 | 0.518 | 0.033 | −1.404 | 0.872 | −0.087 | 0.803 | 1.371 | −0.733 | −0.245 | 0.878 | −0.254 | −0.172 | 0.109 | 0.65 | 0.412 |
| GSTT1 | −0.0955 | 0.3845 | −0.3055 | 0.6015 | 0.4415 | 0.0065 | −0.4745 | −0.2085 | −0.0905 | 0.0955 | −0.3145 | −0.5846 | −1.2545 | −0.0755 | −0.3145 |
| GSTT2 | 0.0295 | −0.9375 | −0.3055 | 0.2425 | −0.7275 | −0.5195 | −0.4875 | 0.2285 | −0.6435 | −0.3775 | −0.2225 | −0.9605 | 0.0135 | 0.0305 | −0.4685 |
| GTF2F2 | 1.323 | 0.636 | −0.212 | 0.219 | 0.176 | 0.253 | 0.053 | 0.084 | −0.384 | −0.434 | −0.287 | 0.162 | −0.73 | −0.34 | −0.458 |
| GTF2I | 0.1955 | −0.1155 | −0.4495 | −0.3455 | 0.3045 | −0.2015 | 0.3075 | 0.4695 | −0.8905 | −0.7405 | −0.1575 | −0.1985 | −0.3845 | −0.0525 | −0.6095 |
| GTF3A | 1.806 | 1.521 | 0.647 | 0.144 | 1.652 | 2.246 | 0.391 | −0.286 | 0.169 | −0.125 | −0.356 | −0.536 | −1.568 | −0.392 | −0.829 |
| GTPBP3 | −1.938 | 0.417 | −3.946 | −0.143 | 0.804 | 0.807 | 0.312 | −2.883 | −0.697 | −0.771 | 0.379 | −0.506 | −2.577 | 0.398 | 0.162 |
| GTPBP4 | −0.357 | 0.342 | 0.907 | 0.903 | 0.08 | 0.78 | 2.194 | 0.368 | −0.981 | −1.084 | −0.175 | −0.998 | −1.151 | −0.946 | −1.421 |
| H1F0 | 0.0325 | −0.5035 | 0.0355 | −0.2735 | −0.0785 | −0.0245 | −0.2055 | 0.3535 | −0.3525 | −0.0595 | 0.7445 | −0.5815 | −0.5635 | 0.0275 | −0.5035 |
| H2AFV | 0.5485 | 1.3885 | 1.2755 | 0.6255 | 0.3435 | 0.5755 | 2.2075 | −0.0075 | −1.2965 | −1.0965 | −0.4665 | −0.0325 | −1.9065 | −0.9775 | −1.6255 |
| H2AFY | −0.4325 | 0.9155 | 0.8815 | −0.2705 | 0.0815 | −0.1245 | 0.9115 | −0.1885 | −1.3505 | −1.6765 | −1.9635 | −0.1315 | −0.8555 | −0.4645 | −0.5295 |
| H2AFZ | −1.1245 | 0.3115 | 0.0805 | 0.0925 | 0.5665 | 0.2325 | 1.6305 | −0.5485 | 0.3585 | 0.6395 | −0.0285 | −0.5165 | −0.5145 | 0.5435 | 0.0135 |
| H2BFS | 0.2405 | 0.6895 | 0.7585 | −0.3835 | 0.6315 | 0.7605 | 1.0245 | 0.1725 | 0.2185 | −0.0415 | 0.3025 | −0.0955 | 0.1965 | −0.0315 | −0.0805 |
| H3F3B | −1.554 | −0.159 | −0.069 | −0.602 | −0.224 | −0.53 | −1.022 | −0.891 | 0.026 | −0.444 | 0.105 | −0.312 | −0.282 | −0.389 | −0.083 |
| H41 | −1.2195 | 1.9695 | −1.1605 | 0.5805 | −0.5775 | −0.4295 | −0.0965 | 0.3965 | 1.8365 | 1.3685 | 0.7595 | −0.2805 | 0.4285 | 0.3845 | 0.4585 |
| HAGH | −0.679 | 0.17 | 0.43 | −0.192 | 0.458 | −0.036 | 0.408 | −0.702 | −0.485 | −0.25 | 0.216 | 0.345 | −0.703 | −0.007 | −0.344 |
| HBG1 | 1.0516 | 1.6945 | 1.3065 | 1.2525 | 1.3915 | 0.1745 | 2.1825 | 0.4485 | −0.8775 | −2.2865 | 0.0005 | −0.9745 | −1.3115 | −0.5445 | −1.6175 |
| HBL01 | 0.05 | 1.542 | 1.302 | 1.182 | 1.094 | 2.633 | 0.254 | 1.092 | −0.737 | 0.031 | −0.062 | 0.15 | −0.591 | −0.068 | 0.056 |
| HCAP-G | 1.8535 | 0.9385 | 0.7875 | 1.2655 | 1.0345 | 0.9825 | 1.5505 | 1.2535 | −0.6975 | −0.4165 | −0.5305 | −0.7335 | −1.2635 | 0.1835 | −0.5275 |
| HCP5 | 0.777 | −0.338 | −0.359 | −0.024 | 0.849 | −1.154 | −0.446 | −0.158 | 0.889 | 1.405 | 1.357 | −0.183 | 0.713 | −0.227 | 0.513 |
| HDAC2 | 0.888 | −1.173 | −0.833 | −0.562 | −0.905 | −0.202 | −1.41 | 1.006 | 0.178 | −0.212 | 0.09 | 0.279 | −0.87 | −1.005 | −0.405 |
| HDC | 0.0305 | 1.2475 | 0.6295 | 0.3765 | −0.1835 | −0.0515 | 0.0345 | 0.0165 | 1.1025 | 0.2666 | 0.9805 | −0.0035 | 1.0665 | −0.0615 | 0.0705 |
| HEBP1 | −0.704 | −1.596 | −1.117 | −1.142 | −0.668 | −1.173 | −2.188 | 1.244 | 1.263 | −0.037 | 0.23 | −1.84 | 0.691 | 0.554 | −0.189 |
| HIPK2 | −0.426 | 0.125 | −0.189 | 0.207 | 0.099 | 0.214 | −0.687 | −1.104 | 0.227 | −0.27 | 0.553 | −0.583 | −1.254 | −0.878 | −0.937 |
| HIPK2 | −1.1055 | −0.4335 | 0.7335 | −0.8195 | −0.3525 | −0.0455 | 1.2355 | −0.5625 | −1.5795 | −2.2085 | −1.3505 | −0.0925 | −0.3105 | −0.5225 | −1.0475 |
| HIS1 | −1.3975 | 0.2735 | 0.2625 | −0.8215 | −0.5145 | −0.3355 | 0.8225 | −0.5065 | −1.3355 | −1.3625 | −1.7425 | −0.6145 | −0.6886 | −0.6785 | −0.8075 |
| HIST1H2A | −1.567 | 0.433 | 0.321 | −0.683 | −0.625 | −0.351 | 0.838 | −0.002 | −1.129 | −1.381 | −1.295 | −0.888 | −1.084 | −0.511 | −0.702 |
| HIST1H2B | −1.5825 | 0.2195 | 0.4015 | −0.6485 | −0.6395 | −0.3805 | 0.8825 | −0.5465 | −1.1475 | −1.2635 | −1.1585 | −0.7435 | −0.4415 | −0.3885 | −0.8205 |
| HIST1H2B | −1.307 | 0.264 | 0.231 | −0.77 | −0.398 | −0.308 | 0.914 | −0.811 | −0.848 | −1.168 | −1.241 | −0.787 | −0.488 | −0.481 | −0.861 |
| HIST1H2B | −0.9505 | 0.5315 | −0.4155 | 0.0685 | 0.2725 | 0.0325 | 1.5395 | −0.1916 | 0.2795 | 0.5815 | −0.1875 | 0.0815 | −0.3215 | 0.1105 | 0.0235 |
| HIST1H2B | 0.3495 | 0.4645 | 0.1235 | −0.4385 | 0.1465 | −0.2715 | −0.9325 | −0.2295 | −0.2265 | −0.9105 | 0.0775 | −0.4785 | −0.9255 | 0.0165 | −0.4165 |
| HIST1H3C | 1.0035 | 0.9365 | 0.8095 | 0.8035 | 0.8345 | 0.3415 | 1.7925 | 1.2345 | −0.1755 | −0.4075 | −0.0735 | −0.4055 | −1.1145 | −0.5345 | −1.0945 |
| HM13 | 0.0205 | 0.7185 | 0.9085 | 0.1565 | 0.3065 | 0.7995 | 2.3505 | 0.2275 | −1.5105 | −1.4385 | −0.2825 | −0.9825 | −1.1235 | −0.9075 | −1.5385 |
| HMGB3 | 0.9035 | 1.0965 | 0.9475 | 0.7815 | 0.7205 | 0.7585 | 1.1255 | 0.4715 | −0.4185 | −0.4065 | −0.1325 | −0.5255 | −0.7945 | −0.3225 | −0.9075 |
| HN1 | 0.103 | 0.557 | 0.17 | 0.401 | 0.677 | −0.162 | 0.64 | −0.117 | 0.68 | 0.738 | 0.259 | −0.635 | −0.653 | 0.894 | 0.047 |
| HNRPA3P | −0.5865 | −0.5465 | −1.0965 | 0.0725 | −0.2515 | −0.6205 | 0.4355 | 0.8395 | 0.8065 | 1.4395 | 2.1945 | 2.1985 | 1.4655 | 1.6085 | 2.3495 |
| HNRPDL | −0.092 | −0.975 | −1.026 | 0.456 | 1.025 | −0.51 | 1.559 | 1.106 | 0.645 | 1.168 | 2.194 | 2.108 | 2.384 | 2.306 | 2.208 |
| HOXA5 | −2.8225 | 0.3765 | −1.5245 | −2.0215 | −0.8895 | −0.1545 | −1.2255 | −1.8785 | −0.9345 | −1.2885 | 0.6935 | 0.0305 | −2.5905 | −2.3295 | −1.2125 |
| HOXA7 | −1.0055 | −0.6215 | −0.8665 | −0.0965 | −0.6315 | 0.2815 | −0.7515 | −0.9865 | 0.3745 | −0.3335 | 0.5265 | −0.2245 | 0.7525 | −0.1555 | 0.0155 |
| HOXB2 | −1.0685 | −0.5815 | −1.6535 | −1.2745 | 0.4275 | 0.8925 | −0.4315 | 0.5725 | 0.0665 | −0.8315 | −0.1885 | −0.4765 | 0.5875 | −1.6275 | 0.3375 |
| HOXB6 | −1.8445 | −0.4095 | −1.0025 | −0.3535 | −1.6775 | 0.4625 | −0.1795 | −0.8415 | 0.3805 | −0.6315 | 0.7805 | −1.0875 | 0.3205 | −0.2895 | −0.8845 |
| HOXB7 | −0.6945 | −0.4305 | −1.0335 | 0.0955 | −0.4485 | −0.3045 | −0.0785 | 0.3935 | −0.5365 | −0.8435 | −0.6025 | −0.2905 | −1.6525 | −0.1625 | −0.1805 |
| HOXC10 | −1.867 | −0.291 | −1.874 | −0.575 | −0.609 | −0.206 | −1.224 | −0.262 | −0.8 | −0.88 | 0.029 | −1.131 | −0.817 | −1.084 | −0.828 |
| HOXC13 | −2.1055 | −0.3725 | −0.3425 | −0.3565 | −1.6495 | −0.7485 | 0.8575 | 0.6795 | 0.0395 | 0.7185 | 0.5715 | 1.3445 | −0.3975 | 1.0325 | 0.6805 |
| HOXC9 | 0.793 | 0.181 | 0.247 | 0.18 | 0.259 | 0.192 | −0.27 | −0.423 | 0.136 | 0.331 | −0.064 | 0.484 | −0.699 | −0.073 | 0.269 |
| HOXD3 | −0.0515 | 0.0185 | −0.4705 | −0.0715 | 0.3285 | 0.3515 | 0.7785 | 0.0165 | −0.7555 | −0.9205 | −0.7785 | −1.1115 | −0.5415 | −0.7595 | −0.5615 |
| HPCL2 | 1.561 | 1.269 | 2.327 | 2.035 | 0.404 | 1.419 | −0.483 | −0.455 | −1.467 | −1.599 | −0.618 | 0.268 | −0.853 | −0.179 | −0.531 |
| HPS3 | 1.1155 | 1.1795 | 0.7625 | 0.2275 | 1.0065 | 1.4565 | 0.2585 | −0.0295 | −0.0405 | −0.2815 | −0.3085 | 0.2085 | −1.0635 | −0.6565 | −0.4105 |
| HRASLS | 1.2265 | 0.7135 | 0.4245 | 0.1245 | 0.3725 | 1.4305 | 0.4545 | 0.2025 | −0.7705 | −1.5085 | −1.3555 | −0.8185 | −1.5765 | −0.6915 | −0.7635 |
| HRB | −1.494 | −0.085 | 0.827 | 1.164 | 0.525 | −1.034 | 0.452 | −0.162 | 0.6 | 0.051 | −0.017 | 1.397 | −0.365 | 1.709 | 0.764 |
| HRSP12 | 0.349 | −0.045 | 0.886 | −0.015 | 1.048 | 0.706 | 1.441 | −0.095 | 0.283 | 0.238 | 0.421 | −0.15 | −0.066 | −0.392 | −0.377 |
| HSD17B2 | 1.6315 | 1.1065 | 0.2615 | −0.3955 | 1.5755 | 0.0155 | 0.8975 | 0.0185 | −0.3705 | −0.3785 | −0.6755 | −0.6835 | −0.8035 | −0.6635 | −0.7815 |
| HSF1 | −1.9095 | −0.7345 | 0.8935 | −0.4205 | 1.1365 | 0.2925 | 0.2625 | −0.9635 | 0.0515 | 0.3475 | 0.6075 | −0.0335 | −0.5725 | 0.0425 | −0.6885 |
| HSPA14 | 0.081 | −0.473 | 0.855 | −0.174 | 0.73 | 0.211 | 0.444 | 0.173 | −1.8 | −1.3 | −0.838 | −1.049 | −2.009 | −0.6 | −1.833 |
| HSPA2 | −0.3495 | 0.0975 | −0.0545 | 0.0875 | 0.2045 | −0.3335 | 0.7825 | −0.6935 | −0.0725 | −0.2825 | 0.1755 | −0.2845 | −0.3615 | −0.4975 | −0.5205 |
| HSPA5BP [symbol] | 0.596 | −0.001 | 0.84 | 0.272 | 0.597 | 1.394 | 1.292 | −0.228 | −0.117 | 0.002 | −0.249 | 0.855 | −0.681 | 0.315 | 0.235 |
| HSPC016 | 0.47 | −0.163 | 0.248 | −0.131 | 0.575 | 0.83 | 1.278 | 0.086 | −0.068 | 0.334 | 0.01 | 0.001 | 0.428 | 0.57 | 0.043 |
| HSPC138 | 1.274 | 1.539 | 0.388 | 0.379 | 0.942 | 0.254 | 1.851 | 0.021 | −1.72 | −1.617 | −0.245 | −0.344 | −2.408 | −1.143 | −1.459 |
| HSPC148 | 1.0005 | 0.2645 | 0.4455 | −0.0025 | 0.8335 | 0.8825 | 1.3925 | −0.5005 | −0.7595 | −0.9725 | −0.5615 | −0.3315 | −0.4335 | −0.8545 | −1.2815 |
| HSPC150 | 0.1325 | −0.4685 | 0.4775 | −0.0315 | 0.4155 | 0.4615 | 1.5405 | 0.3795 | −0.2085 | −0.0085 | −0.1755 | 0.8095 | 0.2295 | 0.3525 | −0.1985 |
| HSPC163 | 2.81 | −0.047 | 0.535 | 0.261 | 1.256 | 0.114 | 0.899 | 0.482 | −0.171 | 0.105 | 0.204 | 1.003 | −0.521 | 0.858 | −0.104 |
| HSPC266 | 0.544 | 0.808 | 0.692 | 0.028 | 0.722 | 1.525 | 1.047 | −0.024 | −0.752 | −0.785 | −0.543 | −0.2 | −0.867 | −0.881 | −0.246 |
| HSU79274 | 1.242 | 0.717 | 0.735 | 0.526 | 0.719 | 0.681 | 0.979 | −0.54 | −0.539 | −0.859 | −0.143 | −0.106 | −0.759 | −1.218 | −0.83 |
| HTATIP2 | −0.334 | 0.01 | 0.593 | −0.015 | −0.274 | 0.036 | 0.313 | −0.371 | −0.287 | −0.451 | 0.239 | −0.265 | −0.282 | −0.293 | −0.584 |
| ICMT | 1.643 | 0.306 | −2.524 | 3.294 | 2.188 | 2.22 | 2.951 | 0.727 | 2.351 | 2.529 | 1.731 | 2.113 | 0.852 | 1.724 | 1.807 |
| ICT1 | −0.081 | 1.192 | −0.265 | 0.546 | 0.349 | 1.495 | 0.738 | −0.085 | −1.146 | −1 | 0.093 | −0.3 | −1.138 | −0.55 | −0.998 |
| ID4 | 0.5295 | −0.2405 | −0.0115 | −0.4515 | 0.4445 | −0.5015 | −0.3335 | 0.0705 | −0.1895 | −0.2615 | 0.0225 | 0.2925 | −0.2876 | −0.8805 | −0.5055 |
| IDH2 | 0.163 | 1.072 | 2.193 | 1.063 | 1.159 | 0.731 | 0.406 | 0.951 | −0.998 | −1.535 | 1.953 | −1.19 | −0.872 | −1.218 | −1.35 |
| IDS | −0.2275 | 1.8465 | 0.9125 | 1.1065 | 2.0185 | 1.1815 | 1.4225 | 0.7015 | −0.6545 | −0.5805 | −1.0825 | −1.2315 | −1.4795 | −1.3535 | −0.7775 |
| IFI30 | 0.2205 | 1.2305 | 0.8525 | 0.2525 | 1.4835 | 0.6995 | 0.5945 | −0.1025 | 0.0295 | 0.1505 | −0.4285 | −0.5715 | −0.1945 | −0.5955 | −0.3085 |
| IFI44 | −1.0935 | 1.2935 | 0.8165 | 0.8455 | 1.4135 | 1.1185 | 0.8915 | −0.4905 | 1.0645 | 0.4175 | 0.6835 | −0.7825 | 0.2525 | −0.8895 | 0.0105 |
| IFIT5 | −0.8725 | −0.4805 | −0.3515 | −0.0985 | −0.3875 | −0.5455 | −0.9785 | 0.0865 | 0.0725 | 0.1255 | 0.2215 | 0.4315 | 0.4515 | 0.0555 | 0.2016 |
| IFITM1 | −0.9225 | −0.5215 | −1.0665 | −0.6385 | −0.0155 | −0.3675 | −1.0395 | 0.3445 | −0.1685 | −0.3485 | −0.3725 | 0.1935 | −0.2335 | −0.8515 | −0.0905 |
| IGBP1 | −4.1805 | −0.8825 | −2.9505 | −0.9575 | −1.8825 | −0.8435 | −2.8735 | −1.2565 | 0.7545 | −0.5095 | 0.5825 | 0.1715 | 0.1225 | 0.4095 | −0.7605 |
| IGFALS | 0.4685 | 2.9555 | 2.3155 | 1.8765 | 1.8075 | 1.4875 | 0.4645 | 1.7985 | 0.7265 | 0.0825 | −0.2965 | −0.0085 | −0.3755 | −0.3305 | −0.3185 |
| IGPBP2 | 1.0805 | 2.6925 | 2.4005 | 1.3855 | 2.5125 | 1.4885 | 0.2875 | 1.5835 | 0.4195 | −0.0855 | 0.3665 | −0.1305 | −0.1215 | −0.2525 | −0.3275 |
| IGHG1 | −3.0205 | 1.8275 | 2.7085 | 0.9435 | 1.2315 | 0.1585 | 0.2405 | 3.4385 | 3.6945 | 4.0275 | 3.4905 | 2.4855 | 1.5295 | 1.2615 | 2.0335 |
| IGHG1 | 0.091 | 3.071 | 3.185 | 0.67 | 2.074 | 3.046 | 0.583 | 1.956 | 0.224 | −0.152 | 0.114 | 1.192 | −1.65 | −0.898 | 0.078 |
| IGJ | 0.171 | 2.352 | 2.1 | 1.048 | 1.391 | 1.742 | 0.261 | 1.255 | −0.21 | −0.097 | 0.445 | 0.839 | −2.22 | −1.345 | −0.197 |
| IGKC | 1.301 | 2.254 | 1.803 | 0.275 | 1.044 | 1.805 | 1.579 | 1.288 | 1.099 | 0.283 | 0.75 | 0.817 | 0.578 | −0.53 | 0.06 |
| IGKC | −0.645 | 2.952 | 2.582 | 0.809 | 1.416 | 1.589 | 0.901 | 2.777 | 1.427 | 1.849 | 0.907 | 0.584 | −0.59 | −0.102 | 0.516 |
| IGLC2 | −0.7895 | −0.4955 | 0.0775 | −0.0715 | 0.3985 | −0.6075 | −0.4035 | −1.2175 | −0.3955 | −0.6165 | −0.0755 | −1.0355 | −0.4865 | −0.7755 | −0.8555 |
| IGLL1 | −0.1306 | −0.0395 | −0.0075 | 0.6285 | 0.5095 | −0.5105 | 0.0985 | −0.0795 | 0.4375 | 0.3935 | 0.3835 | 2.4565 | 0.2025 | 2.3405 | 1.0345 |
| IL13RA1 | 3.684 | 0.584 | 2.511 | 0.437 | 1.878 | 0.695 | 0.071 | 4.335 | −0.294 | 0.205 | 0.791 | 0.381 | −0.593 | −0.155 | −0.078 |
| IL17B | −0.8865 | −1.3795 | 0.0145 | −1.1585 | −0.5095 | −1.8585 | −0.9615 | −1.0045 | 0.0525 | 0.1985 | −0.3415 | −1.9035 | −0.6125 | 0.8385 | 0.8445 |
| IL23A | 0.9585 | 0.8375 | 0.8895 | 1.4025 | 0.5965 | 0.2155 | 0.5265 | 0.4795 | −0.4245 | −0.8355 | −0.1115 | 0.1105 | −0.9495 | −0.1135 | −0.5875 |
| IL6ST | 1.4905 | 1.8375 | −0.3895 | 0.7785 | 1.1055 | 0.9885 | 1.2905 | 1.4025 | 0.8495 | −0.1535 | 1.4125 | 0.2675 | −0.3845 | 0.7815 | 0.6215 |
| ILF2 | 0.289 | −0.665 | 0.107 | 0.115 | −0.054 | −0.744 | −0.512 | −0.867 | −0.391 | −0.241 | −0.305 | −0.419 | −0.921 | −0.114 | −0.37 |
| IMPA2 | −3.7555 | −2.8225 | −3.1105 | −0.9285 | −0.8445 | 0.5395 | −2.7545 | −0.0385 | 2.1175 | −0.3175 | 0.7675 | 0.0875 | 0.7095 | 0.7535 | 1.0275 |
| ING1L | −2.657 | −0.778 | −1.799 | −1.659 | −0.383 | −2.112 | −0.695 | −1.91 | 0.883 | 0.657 | 0.881 | −2.177 | 0.227 | 0.3 | −0.65 |
| INHBB | 0.9455 | 0.2625 | 0.3335 | −0.0235 | 0.4005 | 0.0985 | −0.0375 | 1.0305 | 0.3515 | 0.3265 | −0.3115 | −0.6475 | −0.4885 | −0.0885 | −0.7895 |
| INPP4B | 0.0235 | −0.9855 | −1.7715 | −0.9105 | −0.6845 | 2.4775 | −0.1185 | 0.1175 | −0.2435 | 0.3475 | 0.5515 | 0.0515 | 0.2995 | 0.9785 | 0.2365 |
| INSM1 | −0.92 | −1.37 | −0.182 | −0.846 | −1.388 | −1.033 | −1.31 | −0.198 | −0.591 | −0.384 | −0.317 | −0.876 | −1.277 | −0.287 | −1.047 |
| IQCA | −1.224 | −0.432 | 0.194 | −0.364 | 0.324 | −1.88 | −1.183 | −0.179 | −0.425 | −0.276 | −0.033 | −0.988 | −1.337 | 0.004 | 0.63 |
| IQCE | −0.19 | 2.528 | 2.018 | 0.613 | 1.413 | −0.302 | 0.411 | 2.058 | 0.974 | 0.822 | 1.044 | −0.303 | 1.68 | 0.587 | 0.629 |
| IQGAP2 | −0.1955 | 2.1315 | 3.0575 | 2.7355 | 1.8545 | 1.6325 | 1.4095 | 3.2845 | 0.0325 | 0.2065 | 0.6675 | −0.7315 | 0.5185 | −0.5015 | −0.2485 |
| IRF4 | −0.2005 | 0.4395 | −3.1735 | 0.1405 | −1.1325 | 2.4225 | 1.4385 | 1.5285 | 0.2295 | 2.0805 | 1.1785 | 1.4465 | 1.4785 | 1.7135 | 0.7055 |
| IRTA2 | −0.687 | 2.168 | 1.404 | 0.745 | 1.65 | 0.819 | 0.156 | 0.749 | −0.039 | −0.814 | −0.241 | 0.28 | −0.753 | 0.392 | −0.442 |
| IRX4 | −1.0835 | 0.6935 | 0.2785 | 0.0125 | 0.5485 | 0.9275 | 0.5945 | 0.4385 | −0.2075 | 0.1225 | −0.0295 | −0.4485 | −1.7435 | −0.4225 | −0.4885 |
| ISG20 | 0.263 | −0.554 | 0.913 | 2.163 | −0.005 | 1.695 | 1.25 | 0.052 | 1.171 | 1.267 | 1.933 | 0.38 | 1.951 | 0.929 | 0.657 |
| ISGF3 [symbol] | −1.34 | −0.886 | 0.283 | −0.567 | −0.298 | −1.254 | 0.468 | −1.22 | 0.337 | 0.098 | −0.555 | −0.147 | −0.803 | −0.509 | −0.399 |
| ITGB4 | −0.053 | 0.195 | −0.25 | −0.166 | 0.617 | 1.24 | 0.213 | 3.419 | −0.52 | 0.19 | −0.178 | −1.135 | −0.035 | 0.249 | 0.16 |
| ITPR1 | −0.388 | −2.899 | −0.976 | −1.619 | −3.281 | −3.509 | −2.959 | −2.466 | −0.983 | −0.909 | −0.218 | −0.417 | −0.909 | −0.78 | −0.959 |
| JARID1A | 1.726 | 0.567 | 0.454 | 0.362 | 1.157 | 0.297 | 0.418 | 0.2 | −0.483 | −0.227 | −0.558 | −0.105 | −0.723 | −0.245 | −0.339 |
| KARCA1 | −0.33 | −0.27 | 0.069 | 0.086 | −0.333 | −0.154 | 0.282 | −0.027 | 0.139 | 0.224 | −0.901 | 0.248 | 0.158 | 0.903 | 0.722 |
| KATNA1 | 0.0715 | −0.9425 | 0.2085 | −0.3375 | −0.2505 | 0.0965 | −0.2515 | −0.5725 | −0.4935 | −0.1215 | −1.0935 | −0.5235 | 0.1095 | −0.2315 | 0.2475 |
| KCNC2 | −0.388 | 2.33 | 0.771 | 0.134 | −0.083 | 0.212 | 0.118 | 0.413 | 0.383 | −0.028 | 0.561 | −0.208 | 1.128 | −0.43 | −0.231 |
| KCND2 | −0.173 | 1.559 | 0.157 | 0.739 | 1.535 | 1.698 | −0.374 | 2.307 | 0.316 | 1.244 | 0.867 | 2.139 | 0.069 | 2.252 | 1.761 |
| KCNK4 | −0.438 | −0.485 | −2.579 | −0.508 | −0.08 | 0.062 | −2.5 | −0.938 | −1.247 | −0.397 | −0.822 | 0.075 | −1.029 | −1.158 | −0.958 |
| KCNN4 | 0.1805 | −0.3715 | −1.6825 | −0.3385 | −0.5385 | 0.5755 | −0.6475 | 0.2445 | −0.2655 | −0.4195 | 1.0485 | −0.0205 | −0.1625 | 0.3845 | −0.2945 |
| KCNS3 | −0.7445 | −1.0865 | −2.2525 | −0.8585 | −0.7625 | −0.8365 | −0.9345 | −0.8805 | −0.8615 | −0.7876 | −0.4505 | −1.6895 | −1.4575 | −0.3145 | −0.6355 |
| KCTD15 | 0.089 | −0.002 | 0.857 | −0.611 | 0.422 | 0.473 | 1.347 | −0.557 | −0.149 | −0.723 | −0.421 | −0.298 | −2.999 | −1.878 | −1.791 |
| KCTD3 | 1.251 | 2.142 | 2.282 | 1.987 | 0.661 | 1.083 | 0.765 | −0.311 | 0.264 | 0.058 | 0.218 | −0.758 | −0.008 | 0.795 | 0.18 |
| KDELR2 | 0.4 | 0.529 | 0.459 | 1.203 | −0.102 | −0.117 | 0.854 | −0.892 | −2.419 | −2.28 | 0.05 | 0.242 | −0.9 | −1.292 | −2.435 |
| KIAA0020 | 0.31 | 0.382 | −1.209 | −0.182 | 0.145 | −0.161 | −0.6 | 0.109 | −0.336 | −1.024 | −0.008 | 0.106 | −0.513 | −0.363 | −0.357 |
| KIAA0101 | −1.1285 | −0.4755 | 0.0485 | −1.2285 | −0.6155 | −0.3595 | 0.0855 | −0.0445 | 0.0645 | −0.9205 | −0.8245 | −0.8115 | −1.2905 | −0.3845 | −0.6505 |
| KIAA0153 | 0.2785 | 0.1485 | 0.8675 | −0.1775 | 0.0525 | 0.1265 | −0.3115 | −0.7125 | −0.4385 | −0.6355 | −0.2885 | −1.0295 | −1.5625 | −1.0535 | −0.7395 |
| KIAA0182 | −0.5255 | −0.0885 | 0.4775 | 0.2625 | 0.1855 | 0.4415 | 0.4565 | −0.3845 | −0.2775 | −0.5285 | −0.1435 | 0.7055 | −0.2695 | −0.0935 | −0.2875 |
| KIAA0196 | 0.2825 | −0.1905 | 0.1535 | −0.4946 | −0.4465 | 0.4835 | −0.0216 | −0.4135 | −0.7785 | −0.8195 | −0.8895 | −0.6825 | −2.0835 | −0.9205 | −0.9965 |
| KIAA0241 | 0.22 | −0.073 | 0.287 | −0.237 | 0.316 | 0.183 | 1.031 | 0.012 | −0.818 | −0.258 | −0.473 | −0.332 | −0.579 | 0.001 | −0.328 |
| KIAA0251 | −0.2965 | −0.0055 | −0.0195 | −0.1805 | −0.0225 | 0.1715 | 0.4095 | −0.1585 | −0.2765 | −0.1145 | −0.2715 | −0.3275 | 0.4095 | 0.2055 | −0.0915 |
| KIAA0274 | 0.431 | −0.814 | −0.48 | −0.378 | −0.282 | −1.829 | 0.002 | 0.482 | 0.997 | 1.703 | 0.839 | −0.083 | 1.429 | 0.098 | 0.75 |
| KIAA0515 | 1.0575 | 1.3575 | 1.4355 | 0.5285 | 0.9205 | 1.0325 | 1.1495 | 0.4515 | −0.3065 | −0.2295 | −0.6845 | −1.1255 | −1.0375 | −0.2375 | −0.2405 |
| KIAA0582 | −1.8255 | −1.3265 | −0.1185 | −0.8385 | 0.3385 | −1.9585 | −0.4735 | −0.0385 | −0.6555 | −1.1385 | −0.6145 | −0.5535 | −1.1815 | −0.7765 | −1.1195 |
| KIAA0650 | 0.4625 | −0.8055 | 0.3715 | 0.3145 | 0.9435 | 1.1355 | 1.4485 | −0.3785 | 0.0535 | 0.2475 | 0.3485 | −0.2325 | 0.8125 | 0.4915 | 0.3725 |
| KIAA0703 | 0.823 | −0.48 | −2.3 | −1.06 | −0.496 | 1.138 | 0.489 | 1.28 | −0.771 | 0.108 | 0.684 | −1.192 | 0.239 | −0.402 | −0.85 |
| KIAA0830 | −4.6235 | −3.2985 | −2.6345 | −1.6255 | −2.5535 | −2.7815 | −4.5045 | −0.4155 | −1.4045 | −2.5065 | −1.1185 | 0.5585 | −1.8495 | 1.0335 | −0.5165 |
| KIAA0980 | −0.7295 | −0.7875 | 1.6885 | −0.1915 | 0.0495 | −0.1885 | 0.2935 | −1.0215 | −0.5375 | −0.4715 | 0.1495 | −0.7775 | −0.2035 | −1.0895 | −1.0415 |
| KIAA1324 | −1.42 | −0.447 | −0.673 | 0.07 | −0.789 | −0.442 | −1.228 | 0.297 | 0.207 | 2.683 | 0.434 | −0.054 | 1.21 | 1.523 | 1.053 |
| KIAA1363 | −0.752 | −1.12 | −0.762 | −0.319 | −1.056 | −0.723 | −1.414 | −1.008 | 0.27 | 0.821 | 0.121 | −0.939 | 0.349 | 0.253 | 0.224 |
| KIAA1683 | −0.3725 | 0.4545 | 1.4815 | 0.6355 | −1.1915 | −0.3515 | 1.3385 | −0.0465 | −1.4245 | −1.2075 | −0.4335 | −0.3275 | 0.2515 | −0.3125 | −0.7585 |
| KIF12B | 0.8995 | −0.1365 | −0.2175 | −0.3755 | 1.0385 | 0.9945 | −0.1675 | −0.5705 | −0.7315 | −0.3395 | −0.8285 | −0.9125 | 0.0055 | 0.2205 | −0.2715 |
| KIF24 | 0.6285 | 1.6905 | 0.4535 | 0.4605 | 0.0945 | −0.3525 | 0.0095 | 1.2905 | 0.5935 | 0.2735 | 0.7875 | 0.0825 | 1.0505 | 0.3095 | 0.3425 |
| KIN | −1.0025 | 1.2445 | −1.7475 | 2.1325 | 1.6865 | −0.0365 | 0.5645 | 2.8795 | 1.4395 | 2.2075 | 1.3815 | 3.2315 | 1.1385 | 3.1165 | 4.0995 |
| KIRREL3 | −0.2835 | 1.1435 | −0.5545 | 2.6155 | 1.7275 | 2.5385 | −0.4795 | 0.4095 | 1.4885 | 2.1595 | 2.3435 | 2.1965 | 1.7185 | 1.0885 | 1.0875 |
| KIT | 2.3535 | 0.5765 | −0.2295 | 1.4495 | 0.8465 | 4.0705 | −2.7935 | 2.8865 | 1.1816 | 1.2645 | 2.2355 | 2.9785 | 1.9885 | 1.8175 | 1.5575 |
| KLK5 | 0.852 | −0.424 | −0.454 | 1.351 | −0.189 | 2.865 | −0.788 | 0.929 | 0.608 | 0.727 | 1.376 | 1.707 | 2.387 | 1.155 | 0.365 |
| KLK6 | −0.989 | −0.378 | −1.234 | 2.557 | 1.079 | 2.672 | −0.953 | 2.457 | 1.267 | 1.499 | 2.682 | 2.206 | 2.328 | 1.447 | 0.723 |
| KLK7 | 1.836 | 1.446 | 1.693 | 1.366 | 0.678 | 1.124 | 3.182 | 0.591 | −1.87 | −1.434 | −0.555 | −1.73 | −2.023 | −0.805 | −1.898 |
| KLK8 | 0.6325 | 1.2775 | 1.7735 | 0.8145 | 0.3935 | 1.3835 | 1.7195 | −0.4395 | −0.8295 | −0.7795 | −0.0435 | −0.7805 | −0.8885 | −0.4915 | −0.9575 |
| KNTC2 | 3.715 | 1.482 | 0.131 | 0.247 | −0.811 | −0.479 | −0.121 | −0.072 | −0.301 | −0.343 | −0.495 | 0.408 | 1.26 | 0.13 | 0.179 |
| KPNA2 | −0.7735 | 1.6985 | −0.1725 | 0.3345 | 0.1695 | −0.8715 | 0.0355 | −1.3505 | 0.1435 | 0.0515 | −0.1125 | 0.0055 | −0.2695 | −0.4605 | −0.4905 |
| KREMEN2 | −0.5355 | 2.3015 | 0.1335 | 1.3285 | 1.5965 | 0.8765 | −0.4115 | −0.3245 | 0.2215 | 2.5745 | 2.3895 | 2.7395 | 2.1745 | 3.4565 | 3.3395 |
| KRT10 | 1.3835 | 2.2255 | 0.1025 | 2.0285 | 2.2945 | 0.9615 | −0.0155 | 0.0155 | 1.9675 | 3.2805 | 3.3965 | 2.4305 | 3.0975 | 3.8475 | 3.5415 |
| KRT13 | 1.454 | 2.48 | −1.521 | 2.647 | 1.323 | 1.611 | −0.588 | 0.868 | 4.189 | 2.895 | 3.791 | 0.484 | 1.763 | 1.931 | 1.603 |
| KRT15 | −1.028 | 0.02 | −2.427 | −1.513 | −1.116 | −1.057 | −2.142 | −0.528 | −0.453 | −0.817 | −0.052 | −1.354 | −0.857 | 0.074 | −0.787 |
| KRT17 | −0.3835 | 3.2985 | −3.9255 | 2.9865 | 3.9685 | 2.7065 | 1.5825 | −1.6185 | 2.0585 | 2.2605 | 2.7535 | −0.1955 | 0.2035 | 0.0215 | 0.1115 |
| KRT18 | 1.679 | 0.677 | −2.83 | 3.328 | 1.288 | 1.559 | 0.73 | 2.761 | 3.075 | 2.218 | 3.63 | 0.977 | 0.881 | −0.042 | 0.029 |
| KRT23 | 0.0905 | −0.0375 | 0.0265 | −0.2105 | −0.2315 | −0.3135 | −0.1235 | −0.3345 | 0.1315 | 0.1145 | 0.2995 | −0.1875 | 0.4565 | 0.0805 | −0.3165 |
| KRT6B | −0.519 | 0.228 | −0.319 | −0.118 | −0.035 | −0.389 | −0.167 | −1.035 | −0.22 | −0.308 | 0.484 | −0.156 | 0.108 | −0.24 | −0.296 |
| KRTAP3-3 | −0.7085 | −0.8205 | 0.2745 | 0.0015 | −0.4545 | −0.8155 | −0.5285 | −0.0575 | 1.7785 | 0.9835 | 1.0425 | 1.1545 | 1.0935 | 1.1215 | 0.8255 |
| KRTHA7 | 2.2635 | −2.1455 | 1.7745 | 0.5345 | −0.2395 | 0.4845 | 0.5025 | 1.7445 | 2.1145 | 2.3945 | 1.4545 | 1.4555 | 2.4255 | 2.1046 | 1.6315 |
| LAMA3 | −0.903 | −0.641 | −1.006 | −0.688 | −0.522 | −0.767 | −0.873 | −0.692 | 0.135 | −0.181 | 0.049 | −0.893 | −0.726 | −0.788 | −0.711 |
| LAMB3 | 0.588 | 1.411 | 1.876 | 0.185 | 1.286 | 1.204 | −0.009 | 0.943 | −0.011 | 0.156 | 0.009 | −0.382 | 0.018 | −0.224 | 0.017 |
| LASS2 | 0.5795 | 0.6015 | 0.6795 | 0.7445 | 0.3845 | 0.4015 | 1.9625 | 0.4285 | 0.8735 | 1.0995 | −0.0285 | −0.5605 | 0.1485 | 0.1175 | 0.3985 |
| LAX | −1.9325 | −0.5855 | −1.1685 | −0.9155 | −0.5255 | −1.0365 | 1.2245 | 0.5995 | −0.5985 | 1.0445 | 0.6025 | 0.7815 | 0.6085 | 0.0235 | 0.1215 |
| LBR | 3.5135 | 1.8885 | 1.1605 | 0.3685 | 0.8795 | 1.0005 | 0.7325 | 3.4105 | 1.0205 | 0.4845 | 0.2885 | 1.6355 | −0.1095 | 0.9705 | 0.0185 |
| LCE1B | −2.2905 | 0.7505 | −0.8895 | 0.2235 | −0.3585 | −1.1885 | −0.2855 | −0.2805 | −0.0195 | 0.2735 | −0.3775 | 0.8135 | −1.4595 | −0.3445 | −0.3545 |
| LDHB | −0.242 | 0.031 | −0.75 | −0.22 | −0.284 | 0.309 | −0.78 | 0.773 | 0.003 | 0.272 | 0.009 | 1.016 | 0.26 | 0.459 | 0.812 |
| LDOC1L | −2.06 | −1.748 | −1.979 | −1.031 | 0.441 | −2.183 | −2.09 | −1.445 | −1.551 | −0.575 | −0.54 | 1.16 | −0.269 | −0.367 | 0.254 |
| LETMD1 | −0.9565 | 2.2295 | 2.5195 | 2.4195 | 3.1305 | −2.0495 | 1.2545 | 1.9925 | −1.0495 | 1.8005 | 1.0835 | −0.0765 | 0.7095 | 0.3375 | −0.3545 |
| LFNG | −0.411 | −0.392 | −0.269 | −0.135 | −0.249 | −0.462 | −0.561 | −0.993 | −0.005 | 0.145 | 0.105 | 0.594 | −0.018 | 0.268 | 0.259 |
| LGALS2 | −1.233 | −1.571 | −2.165 | −0.499 | −1.008 | 0.649 | 0.457 | 0.984 | −0.484 | −0.797 | −1.335 | 0.122 | −1.418 | −0.303 | −0.225 |
| LGTN | −2.659 | −1.197 | −2.657 | −1.252 | −2.151 | −0.535 | −2.208 | 1.191 | −0.584 | 0.623 | 0.703 | −1.061 | −0.171 | −1.345 | −0.059 |
| LNX | 0.1825 | −1.0775 | 0.5445 | −0.3835 | 0.3015 | 0.0165 | −0.4785 | −0.4476 | −0.9425 | −0.1255 | −0.3565 | 0.1125 | −1.3855 | −0.7485 | −0.4625 |
| LOC11247 | 0.529 | 0.203 | 0.462 | 0.43 | 0.285 | −0.284 | 0.436 | 0.438 | −0.822 | −0.806 | −0.892 | −0.783 | −0.71 | −0.365 | −0.985 |
| LOC11492 | 0.58 | 0.147 | −0.086 | 0.704 | −1.589 | −0.517 | −2.125 | 0.675 | −0.641 | −0.288 | −0.288 | −0.184 | −1.18 | −0.141 | −0.834 |
| LOC11497 | 0.7665 | 0.4005 | 0.4755 | −0.0345 | 1.7745 | 0.3056 | 1.6185 | 0.1265 | 0.0095 | 0.1795 | 0.0805 | 0.4675 | 0.4075 | 0.2855 | 0.0425 |
| LOC11621 | −3.5935 | −0.6805 | −1.7185 | −0.9825 | −1.5795 | −0.6985 | −1.5245 | −2.2425 | −0.2435 | −1.0805 | 0.0975 | 0.1425 | −0.0565 | −0.2775 | −0.7295 |
| LOC11644 | −4.6865 | −2.7285 | 3.9125 | −1.0305 | −1.7695 | −2.8765 | −4.3405 | −4.0095 | −1.3895 | −2.4265 | −1.0095 | −0.4125 | −2.0935 | −0.4725 | 0.6385 |
| LOC12022 | −0.1915 | −0.1895 | −0.3505 | −0.1575 | −0.4505 | −0.2485 | −0.7455 | −0.0495 | 0.0835 | 0.0185 | 0.2835 | −0.3805 | 1.0425 | 0.0815 | −0.0805 |
| LOC12422 | −1.9245 | −1.7615 | −0.8085 | −0.2835 | −1.5425 | 0.2225 | −1.0855 | −0.1475 | 0.0355 | 0.8585 | −0.3085 | −1.3845 | 1.0535 | 0.2375 | 0.3435 |
| LOC14654 | −1.48 | −1.042 | −0.824 | −1.001 | 0.005 | −1.568 | −1.887 | −1.224 | −1.252 | −1.204 | −1.236 | −0.392 | −2.074 | −0.798 | −1.46 |
| LOC15506 | 0.713 | 0.365 | −1.055 | −0.033 | −0.467 | 0.142 | −0.221 | 0.531 | −0.113 | −0.865 | 0.385 | 1.178 | 0.332 | 0.273 | 0.458 |
| LOC20189 | −1.6195 | −1.0115 | −1.4775 | 2.8385 | 1.2245 | 1.6885 | 3.7555 | −0.1535 | 0.4775 | 0.9395 | 1.2465 | 2.8725 | 0.5975 | 1.7805 | 1.2785 |
| LOC25398 | −0.35 | −0.126 | −0.474 | 0.016 | −0.402 | 0.628 | 0.522 | −0.256 | −0.387 | −0.329 | −0.475 | −0.408 | 0.189 | 0.183 | −0.32 |
| LOC33990 | −2.436 | −1.587 | −2.888 | −1.295 | −1.26 | −1.425 | −2.082 | −1.462 | −0.999 | −1.376 | −0.465 | −0.424 | −1.017 | −0.439 | −0.672 |
| LOC34817 | −0.6645 | −1.1615 | −1.0085 | −0.0595 | −0.5595 | 0.5255 | −0.1195 | −0.5605 | −0.7685 | −0.7635 | −0.6325 | 0.4325 | −0.5525 | −0.1555 | 0.2815 |
| LOC40045 | 0.66 | 0.68 | 0.193 | 0.567 | 1.64 | −0.527 | 0.26 | 0.065 | −1.189 | −0.833 | −1.015 | 0.28 | −0.93 | −1.131 | −0.55 |
| LOC44089 | 0.07 | 0.697 | 0.444 | 0.347 | 0.911 | 0.997 | 0.771 | −0.359 | −0.58 | −0.396 | −0.446 | −0.298 | −1.204 | −0.53 | −0.377 |
| LOC49386 | −0.614 | −1.495 | −1.537 | −0.728 | 0.388 | 0.358 | 0.131 | −0.490 | 0.317 | 0.566 | 0.584 | 0.196 | 0.27 | −0.064 | 0.209 |
| LOC51123 | −0.381 | 0.019 | −0.007 | −0.16 | −0.159 | 0.921 | 0.612 | −0.848 | −0.587 | −0.501 | −0.288 | −0.375 | −0.462 | −0.252 | −0.254 |
| LOC51161 | −2.1305 | −0.0285 | −1.5705 | −1.2075 | −0.9805 | −1.4665 | −0.8715 | −1.1465 | −1.1695 | −1.0585 | −0.1165 | −0.8705 | −0.3355 | −0.6295 | −0.8895 |
| LOC51321 | 1.0555 | 0.4695 | 0.9275 | 0.5705 | 0.1665 | −0.7205 | −0.2385 | 0.7815 | 0.1975 | 0.7826 | −0.1205 | 1.0395 | 0.8705 | 0.8795 | 0.4155 |
| LOC51760 | 4.5815 | 0.4495 | −0.3745 | −0.5265 | −0.1465 | −0.1975 | −1.2705 | 0.7685 | −0.2115 | −0.2705 | −0.0145 | 0.0455 | −0.2055 | −0.4285 | 0.3905 |
| LOC81558 | 0.145 | −1.089 | −0.785 | −1.124 | −1.501 | −1.082 | −1.537 | −0.263 | −1.309 | −1.334 | −1.229 | −1.145 | −1.489 | −0.735 | −1.139 |
| LOC81569 | 1.4345 | 0.9095 | 1.2275 | 0.2635 | 0.2815 | −0.8875 | −0.6445 | 0.8295 | −0.2875 | −0.3055 | −0.1055 | −0.3275 | −0.2275 | −0.3585 | −0.5375 |
| LOC90355 | −0.399 | −0.594 | −0.393 | 0.734 | −0.118 | 0.181 | −0.932 | −1.16 | −0.899 | −1.458 | −0.908 | 0.098 | −1.616 | −0.988 | −1.444 |
| LOC90701 | −2.34 | −0.975 | 2.078 | 0.892 | 1.897 | 0.736 | −1.43 | 1.727 | 0.007 | 0.582 | 0.131 | −1.575 | −0.78 | −0.007 | −0.949 |
| LONP | −0.2895 | −0.2495 | −0.2565 | −0.8135 | 0.1585 | 0.5925 | −0.2915 | −1.0605 | −0.7045 | −0.4385 | −0.5085 | −0.6765 | −1.7145 | −0.2935 | −0.3825 |
| LRAP | −1.0675 | −1.2385 | −0.1876 | −0.7445 | −0.4585 | −0.9035 | −1.0925 | −1.3325 | 0.2015 | 0.0075 | 0.6305 | 0.1965 | −0.2585 | 0.8205 | 0.5985 |
| LRBA | 1.7975 | −0.5395 | −1.1045 | −0.1125 | −1.0715 | 0.7325 | 0.3185 | 2.1705 | 0.0245 | 0.1945 | −0.2885 | −0.8935 | 0.5745 | −0.0035 | 0.2885 |
| LRIG1 | 1.4815 | 1.1765 | 1.4535 | 1.3605 | 0.8465 | 0.2585 | 0.1995 | 1.1605 | −0.5365 | −1.3045 | −0.5035 | 0.1395 | −1.0065 | −1.7715 | −1.3155 |
| LRP6 | −1.198 | −0.784 | −2.683 | −0.582 | 0.464 | −1.942 | −1.065 | −0.555 | −0.444 | 1.789 | −0.384 | 0.591 | 0.248 | −0.41 | 0.471 |
| LRP8 | −0.705 | −0.888 | −0.811 | −0.922 | −0.428 | −0.895 | −1.013 | −0.941 | −0.814 | −0.148 | −0.672 | −1.629 | −1.229 | 0.017 | −0.153 |
| LRRC17 | 1.1235 | 0.1625 | 0.9545 | 1.6235 | 0.5105 | 0.9345 | 0.7845 | −0.2015 | 0.0095 | −0.0945 | −0.0135 | −0.0635 | −0.6525 | −0.0705 | −0.3435 |
| LRRC2 | 1.3105 | 0.0385 | 0.7046 | 0.2055 | 0.6285 | −0.3795 | 1.0745 | 0.5445 | −0.4325 | −0.2545 | 0.0075 | −0.3895 | −0.2215 | −0.5405 | −0.5975 |
| LSM1 | 3.3215 | 0.6405 | 2.1975 | 1.7515 | 3.8115 | 0.1675 | 1.7225 | 2.0235 | −1.3945 | 0.6405 | 0.8885 | −0.9275 | −1.2435 | −0.6945 | −0.4005 |
| LSM8 | 0.748 | 1.497 | 0.026 | 1.163 | 1.551 | 4.28 | 1.198 | 0.81 | 0.805 | −0.068 | 0.084 | 1.897 | 0.251 | 1.028 | 0.36 |
| LTB | −0.143 | 1.141 | 2.243 | 1.068 | 1.848 | 1.244 | 1.169 | 1.01 | −0.011 | 0.346 | 1.48 | 0.605 | −0.858 | −0.402 | 0.348 |
| LY6D | 1.647 | 1.028 | 1.755 | 0.945 | 0.504 | 0.875 | 2.933 | 0.015 | −1.865 | −1.985 | −1.235 | 0.28 | 1.076 | −1.025 | −1.482 |
| LYN | 0.304 | 0.652 | 0.048 | −0.173 | 0.085 | −0.251 | −0.319 | −0.142 | −0.572 | −0.099 | −0.422 | −0.841 | −0.572 | 0.085 | −0.152 |
| MA02L1 | −0.123 | 1.898 | −0.317 | −0.188 | −0.012 | 0.198 | −0.373 | 0.193 | 1.092 | 0.568 | 1.027 | 0.402 | 0.177 | 0.567 | 0.772 |
| MADP-1 | −1.9855 | 2.6035 | −0.2755 | 0.1635 | −0.7075 | 0.9725 | −0.0795 | 0.3365 | 0.8735 | 0.3275 | 0.3225 | 0.0935 | 0.6765 | 0.5895 | 0.1705 |
| MAGEA12 | −0.35 | 2.35 | −0.418 | −0.13 | 0.12 | 0.53 | 0 | 0.781 | 1.031 | 0.423 | 0.907 | 0.25 | 0.35 | 0.145 | 0.31 |
| MAGEA2 | −1.8775 | 2.7775 | 0.0225 | 0.4485 | −1.6815 | 0.7375 | 0.0695 | −0.4325 | 0.7985 | 0.6175 | −0.0655 | −0.0835 | 0.6875 | 0.9875 | −0.1695 |
| MAGEA3 | 0.8565 | 0.7595 | −1.0925 | 0.0105 | −0.6905 | 1.2775 | 1.8425 | 0.7325 | 0.2625 | 0.5845 | 1.0315 | 0.8015 | 0.5075 | 1.2495 | 0.8595 |
| MAGEA4 | 1.679 | 3.045 | −0.028 | 1.564 | 1.849 | 1.179 | 0.397 | 0.515 | 0.104 | 1.373 | −0.105 | 1.192 | −0.029 | 0.501 | −0.229 |
| MAGED4 | 0.4695 | −0.8525 | 0.1565 | 0.0665 | −0.2525 | −0.3055 | −0.4685 | −0.0895 | −0.3135 | −0.5005 | −0.2175 | −0.7335 | −0.6885 | 0.2235 | −0.2895 |
| MAGI1 | 1.8225 | −1.2585 | 0.8725 | 0.6005 | −0.3785 | −0.1945 | 0.0885 | 0.1875 | −1.6305 | −2.1525 | −0.4805 | −1.1085 | −2.3045 | −1.6715 | −1.8805 |
| MAGI-3 | 0.384 | −0.019 | 0.662 | 0.536 | 0.695 | 0.07 | −0.248 | 0.588 | −0.121 | −0.024 | 0.818 | −0.088 | −0.048 | −0.274 | −0.058 |
| MAL2 | 0.264 | −0.746 | −0.225 | 0.405 | −0.27 | −0.677 | −0.005 | −1.336 | −0.692 | −0.852 | 1.216 | −0.843 | −0.107 | −0.694 | −1.222 |
| MAN2B1 | 3.8015 | 0.8685 | 2.8965 | 0.4065 | 3.1725 | 2.4045 | 1.9355 | 1.3455 | 3.1975 | 1.3475 | 2.0945 | 1.2565 | 0.0275 | −0.6025 | 0.1315 |
| MAPK8IP2 | −0.4295 | 0.6975 | 1.7765 | 0.8545 | 1.1175 | 0.2365 | −0.0325 | 1.4805 | −0.4205 | −0.6086 | −0.4185 | −1.0685 | −1.2235 | −0.5995 | 0.0995 |
| MARCO | −2.033 | −0.471 | −0.869 | −0.02 | −1.599 | −0.93 | −1.885 | −1.508 | −0.318 | −0.805 | 0.337 | 0.245 | 0.091 | 1.102 | 0.264 |
| MARLIN1 | 1.041 | 1.042 | 1.136 | 0.941 | −0.3 | −0.313 | 1.49 | 0.58 | 0.421 | 0.234 | 0.162 | −0.05 | 0.18 | 0.451 | −0.166 |
| MASS1 | 1.1216 | 1.4945 | 0.7465 | 1.1075 | 0.0385 | 0.8325 | 0.5265 | 1.3135 | −0.1276 | 0.1745 | 0.2155 | 0.2275 | −0.4845 | 0.1595 | −0.1485 |
| MCM2 | −0.8665 | 1.5165 | −1.6295 | 0.0715 | 2.5285 | 1.4145 | −0.5885 | 0.2275 | 0.2135 | −0.0355 | 0.7285 | 2.3875 | −0.5865 | 0.1605 | 1.2655 |
| MCM3 | −2.771 | −1.691 | −1.304 | −1.415 | −2.072 | −1.609 | −2.655 | −1.707 | −1.226 | −1.865 | −0.739 | −0.775 | −1.444 | −0.825 | −0.806 |
| MESP1 | 0.7915 | 1.2365 | −2.4885 | 0.8055 | 0.9215 | 1.1185 | 0.4725 | 0.8465 | −0.3875 | −0.6805 | −0.5435 | −0.6525 | −1.4925 | −1.6035 | −1.1765 |
| METRN | 0.2195 | 0.0025 | 0.3525 | 0.3935 | 1.0445 | 0.2935 | 1.6725 | −0.4165 | −0.2935 | −0.1365 | −0.4165 | 1.3145 | 0.4685 | 0.7765 | 0.7035 |
| MFAP2 | 0.583 | 0.236 | 0.038 | −0.414 | 0.053 | 1.268 | −0.175 | 0.153 | −0.177 | −0.19 | 0.345 | −0.421 | −1.194 | −0.340 | −0.455 |
| MFGE8 | −0.4575 | −0.3195 | 0.0005 | −0.3075 | 0.0055 | 0.0695 | 0.0455 | 0.6445 | −0.8345 | −0.7935 | 0.2375 | −0.4585 | −0.4555 | −0.4405 | −0.7645 |
| MGAT4B | −0.46 | 0.694 | 0.508 | 0.591 | 1.246 | 0.01 | 0.629 | 1.038 | 0.522 | 0.914 | 0.703 | −0.518 | 1.065 | 0.15 | −0.494 |
| MGC1091 [symbol] | −0.9255 | −1.2825 | −2.1955 | −0.7195 | −2.3585 | −2.8445 | −3.6965 | −2.0125 | −2.0375 | −1.4675 | −0.2885 | −0.2645 | −1.0445 | −0.8925 | −1.5345 |
| MGC1098 [symbol] | 1.6005 | −0.0545 | −0.8595 | 0.3305 | −0.1455 | −0.4445 | 1.6295 | 1.2605 | −0.3885 | −0.1435 | 0.2285 | 0.8305 | −0.4875 | 0.3355 | 0.7075 |
| MGC1124 [symbol] | 1.075 | 0.079 | −0.311 | 0.413 | 0.743 | 0.746 | 0.888 | −0.142 | 0.43 | 0.644 | 0.53 | 0.268 | 0.348 | 0.426 | 0.133 |
| MGC1127 [symbol] | 0.776 | 0.926 | 0.212 | 0.196 | 0.539 | 1.001 | 0.838 | 4.383 | −0.201 | 0.094 | 0.12 | −0.732 | 0.438 | 0.13 | 0.002 |
| MGC1312 [symbol] | −1.064 | −1.167 | −1.291 | −1.068 | −0.663 | 0.057 | −0.486 | −0.938 | −0.548 | −1.082 | −0.149 | 0.09 | −1.087 | −0.545 | −0.515 |
| MGC1318 [symbol] | 0.328 | 0.038 | 0.45 | −0.106 | 0.745 | 0.34 | −1.245 | −0.292 | 0.374 | −0.436 | 0.057 | 0.32 | 0.168 | −0.059 | 0.208 |
| MGC1414 [symbol] | 0.3875 | 0.2445 | 0.5185 | 0.0485 | 0.4945 | −0.1285 | −0.7765 | 0.3505 | −0.0475 | −0.2855 | 0.0035 | −1.1235 | −0.2105 | −0.0135 | −0.5325 |
| MGG1428 [symbol] | 0.248 | 0.303 | 0.161 | −0.386 | 0.693 | 0.356 | −0.433 | 0.814 | −0.279 | −0.663 | −0.531 | −0.157 | −0.91 | −0.829 | −0.587 |
| MGC1481 [symbol] | 2.159 | 0.492 | 2.627 | 0.45 | 1.649 | 1.875 | 1.203 | 2.069 | 0.023 | −0.016 | 0.094 | −0.43 | 0.398 | 0.313 | −0.903 |
| MGC1580 [symbol] | −0.0745 | 0.0715 | −0.0255 | −0.2615 | −0.0745 | 0.6655 | 0.6735 | 0.3645 | −0.3225 | −0.4785 | −0.5005 | 0.2455 | −0.0145 | −0.2845 | −0.1555 |
| MGC1663 [symbol] | −0.038 | 0.82 | 0.491 | 0.557 | 0.438 | 0.078 | 1.147 | 0.79 | 0.22 | 0.178 | 0.458 | 0.171 | −1.07 | −0.6 | −0.312 |
| MGC1873 [symbol] | 1.519 | 0.262 | −0.107 | 2.005 | 1.374 | 1.229 | 1.308 | 0.178 | −0.558 | −0.808 | 0.064 | 0.048 | 1.112 | −0.285 | −0.159 |
| MGC2053 [symbol] | −0.022 | −0.39 | −0.919 | −0.833 | 0.48 | 0.163 | −0.485 | −0.406 | −0.376 | −0.08 | −0.369 | −0.056 | −0.544 | −0.003 | −0.231 |
| MGC2398 [symbol] | 1.958 | 0.275 | 0.738 | 0.67 | 0.765 | 1.358 | 1.852 | −0.5 | −0.034 | −0.135 | −0.342 | 0.629 | −2.033 | −0.183 | −0.533 |
| MGC2610 | 0.533 | 2.6 | 1.808 | −0.033 | 0.758 | 1.738 | 0.269 | 1.328 | 0.751 | 0.859 | −0.165 | 0.361 | −0.16 | −0.196 | −0.178 |
| MGC2714 | 0.951 | 3.138 | 2.57 | 0.627 | 1.507 | 2.105 | 0.327 | 3.157 | 0.518 | 0.449 | −0.184 | 0.768 | −0.42 | −0.753 | 0.068 |
| MGC2716 [symbol] | 1.1905 | 3.2505 | 2.4455 | 0.7715 | 1.4545 | 1.7385 | 0.3325 | 3.5505 | −0.0445 | −0.2135 | −0.0565 | 0.6505 | −0.5645 | −0.7315 | 0.1165 |
| MGC2716 [symbol] | 0.3285 | 2.4075 | 2.5755 | 0.4535 | 1.5045 | 2.5135 | 0.8805 | 1.4555 | 0.8005 | 1.4765 | −0.0185 | 0.8285 | −0.8295 | −0.9065 | −0.1255 |
| MGC2716 [symbol] | 1.401 | 2.885 | 2.53 | 0.511 | 1.23 | 1.887 | 0.178 | 3.107 | 0.885 | 0.645 | −0.323 | 0.438 | 0.306 | −0.62 | 0.945 |
| MGC2716 [symbol] | 1.4495 | 0.4085 | 0.8825 | 0.3115 | 0.3535 | −0.1285 | 0.3495 | 0.4755 | −0.7395 | −0.9215 | −0.5775 | 0.2685 | −1.2565 | −0.3295 | −0.3285 |
| MGC2716 [symbol] | −1.585 | 0.048 | −0.886 | −0.358 | −0.188 | −1.087 | −1.683 | −1.401 | 0.114 | 0.624 | −0.534 | 0.454 | 0.149 | 0.188 | 0.673 |
| MGC2887 [symbol] | 0.181 | −0.363 | −0.238 | −0.069 | −0.831 | 1.12 | −0.503 | 1.642 | −0.813 | −0.686 | 0.427 | −0.768 | 0.258 | 0.238 | −0.232 |
| MGC3212 [symbol] | 1.201 | −0.193 | 0.78 | 0.83 | 2.645 | 2.002 | 0.918 | −0.386 | 1.099 | 1.684 | 2.071 | 1.332 | 1.219 | 1.274 | 0.214 |
| MGC3321 [symbol] | 0.1705 | 0.7265 | 1.6735 | 0.0935 | 0.5855 | 1.2876 | 1.9955 | 1.4535 | 0.2365 | −0.5785 | −0.2455 | −0.0885 | −0.3825 | −0.3235 | 0.4635 |
| MGC3484 [symbol] | 0.8335 | −0.3525 | −0.3455 | 0.6415 | −0.8865 | 0.4545 | −0.3845 | 1.5095 | −1.1515 | −0.8215 | −0.2105 | −0.9115 | 0.8365 | 0.6116 | −0.1015 |
| MGC3492 [symbol] | −0.701 | −0.265 | −0.77 | −0.39 | 0.203 | 0.209 | −1.104 | −0.951 | 0.162 | −0.112 | 1.377 | 0.814 | −0.482 | 1.363 | −0.224 |
| MGC4021 [symbol] | 1.777 | 0.001 | 0.854 | 0.612 | 0.889 | 0.337 | 0.599 | −0.315 | −0.245 | −0.179 | −0.039 | 0.275 | −0.531 | −0.366 | −0.523 |
| MGC4251 | 0.6175 | 0.3385 | −0.0665 | −0.3945 | −0.7165 | −0.1385 | −0.7375 | −0.2725 | −0.6725 | −0.3185 | 0.0065 | −0.5785 | −0.0105 | −0.3735 | −0.6315 |
| MGC4308 | −0.583 | −0.83 | −1.955 | −0.608 | −0.08 | 0.310 | −0.685 | −0.427 | −0.062 | −0.302 | 0.104 | 0.207 | −0.453 | −0.334 | −0.1 |
| MGC4800 | 3.7445 | 1.8245 | 1.0835 | 3.7215 | 2.0545 | 3.5635 | 0.0745 | 3.6135 | 1.5755 | 1.2975 | 2.4585 | 3.0845 | 2.8645 | 2.4005 | 1.5545 |
| MGC4659 | 0.942 | 0.528 | 1.108 | 1.386 | 0.784 | 1.809 | 2.224 | 0.565 | 0.73 | 1.008 | 0.648 | 0.536 | 0.254 | 0.759 | 0.772 |
| MIA | 1.0345 | 0.9595 | 1.3925 | 1.5275 | 1.1855 | 2.1125 | 2.5085 | 1.1165 | 0.5715 | 1.0255 | 0.3635 | 0.0175 | −0.1105 | 0.9885 | 1.1105 |
| MID1 | 1.6075 | 0.6815 | 2.1615 | 2.2845 | 0.8835 | 1.9705 | 2.7135 | 0.5395 | 1.2595 | 1.9105 | 0.9095 | 0.7745 | 1.6965 | 1.4345 | 1.3225 |
| MID1 | 0.990 | 0.156 | 0.954 | 0.122 | −0.004 | −0.999 | 1.449 | 0.124 | −1.529 | −1.931 | 0.341 | −0.996 | −1.615 | −0.682 | −1.43 |
| MID1 | 0.1095 | 0.0725 | 0.0975 | −0.2065 | 0.3955 | 0.8935 | 0.0085 | 1.4185 | −0.3875 | −0.3055 | −0.0085 | 0.0655 | −0.9585 | −0.8315 | 0.2285 |
| MLF1IP | 3.5585 | 1.2735 | −0.9655 | 4.8635 | 3.2216 | 5.0265 | −0.3885 | 0.0255 | −0.1605 | −0.7505 | −0.4495 | −1.2335 | −1.2045 | −0.5155 | 0.0795 |
| MLSTD1 | 3.422 | −1.665 | 2.272 | 1.028 | 2.311 | 1.732 | 1.541 | 0.658 | 0.488 | 1.702 | 3.629 | 1.082 | 0.677 | −0.05 | 0.381 |
| MMP1 | 0.005 | −0.13 | 0.049 | 0.341 | 0.334 | 0.078 | 0.203 | −1.2 | −0.477 | −0.532 | −0.842 | −0.549 | −0.78 | −0.441 | −0.601 |
| MMP7 | 0.1495 | 1.1455 | −0.0145 | −0.0725 | 0.8165 | 1.2315 | −0.9625 | −0.6045 | −0.5475 | −0.9175 | −0.7255 | 0.1385 | −1.1155 | −0.1475 | −0.6465 |
| MNAT1 | 1.5875 | 1.0945 | 1.0265 | 0.4105 | 1.7225 | 0.7265 | 0.9975 | 0.1825 | −1.0155 | −1.0885 | −0.4715 | 0.5425 | −1.3455 | −0.5965 | −0.7715 |
| MRPL14 | −0.97 | 0.677 | 0.155 | −0.311 | 0.58 | −0.001 | −0.105 | −0.184 | −0.249 | −0.297 | −0.045 | 0.272 | −0.506 | −0.099 | −0.162 |
| MRPL15 | 0.0085 | 0.0285 | 0.1585 | −0.0335 | −0.0325 | −0.0615 | 0.4375 | 0.2795 | 0.1435 | −0.1815 | 0.0335 | 0.4695 | −0.6175 | −0.1135 | −0.0005 |
| MRPL27 | 0.2275 | −1.0495 | −0.7105 | −0.5335 | 0.1795 | −0.0995 | −1.1445 | −0.8935 | −0.3795 | −0.2795 | −0.2505 | 0.6495 | −0.5385 | −0.9265 | −0.2725 |
| MRPL34 | 0.424 | 0.927 | −0.328 | −0.047 | 0.231 | −0.286 | −1.263 | −0.508 | −0.477 | −0.517 | −0.195 | 0.403 | 0.069 | 0.497 | −0.337 |
| MRPL43 | 0.7715 | −0.1425 | 0.0805 | 0.2985 | 0.7255 | −0.1885 | 0.3985 | 0.9445 | 0.0025 | −0.1895 | 1.2045 | 0.6395 | −0.3825 | 0.0555 | −0.3285 |
| MRPL45 | 0.791 | 0.704 | 1.013 | 0.236 | 0.259 | 0.022 | −0.013 | 0.23 | −0.36 | −0.233 | −0.155 | 0.073 | 0.07 | 0.144 | −0.327 |
| MRPL48 | 1.1795 | 0.0225 | 0.6895 | −0.5175 | 0.4885 | −0.3165 | 0.1235 | −0.3495 | −0.9215 | −1.3825 | −0.3195 | 0.7005 | −1.1095 | −1.0685 | −1.1265 |
| MRPL9 | 0.2555 | 0.4545 | −0.1975 | −0.0095 | 0.2035 | 0.7685 | 0.4815 | 0.5105 | 0.1305 | −0.6815 | 0.0885 | 0.4925 | −0.1245 | −0.3525 | −0.1435 |
| MRPS14 | 0.515 | −0.386 | 0.271 | 0.655 | −0.227 | 0.366 | 0.162 | 0.34 | −1.035 | −1.821 | −0.696 | −0.104 | −1.071 | −0.928 | −1.414 |
| MRPS15 | −0.194 | 0.053 | 0.63 | 0.268 | −0.169 | 0.389 | 0.477 | 0.523 | −0.147 | −0.229 | −0.07 | 0.385 | 0.029 | −0.085 | −0.3 |
| MRPS17 | 0.544 | 0.225 | 0.587 | 0.391 | 0.289 | 0.475 | 0.44 | −0.293 | 0.095 | −0.818 | 0.571 | 0.843 | −0.448 | −0.314 | −0.226 |
| MRPS24 | −0.7735 | 0.3785 | 0.0435 | 0.4495 | 0.2445 | −0.0185 | −0.3095 | −0.0045 | −0.4125 | −0.2235 | −0.4305 | −0.2555 | 0.0595 | 0.2305 | −0.0785 |
| MRPS25 | −0.418 | −0.086 | 0.011 | 0.078 | −0.591 | 0.695 | 0.048 | −0.397 | 0.377 | 0.088 | 0.374 | 0.077 | −0.07 | −0.842 | −0.147 |
| MRPS31 | −0.174 | 0.534 | 2.334 | 2.037 | 2.415 | −0.113 | 1.575 | 0.987 | 0.222 | 0.295 | 0.241 | 0.053 | 0.519 | −0.63 | −0.823 |
| MRPS6 | 2.0105 | 1.0435 | 0.9025 | 1.0876 | 0.0725 | 0.3595 | 0.2775 | 0.3915 | −0.6985 | 0.0215 | −0.1935 | −1.4005 | −0.6595 | −0.1475 | −0.4795 |
| MS4A1 | 0.6755 | −0.3725 | 0.2315 | −0.1475 | −0.1605 | −0.2445 | 0.0295 | −0.1695 | −0.2255 | −0.3185 | −0.4885 | −1.0845 | −0.1155 | 0.4125 | −0.0245 |
| MSH2 | −0.066 | −0.519 | −1.129 | −0.558 | −1.446 | −1.353 | −0.861 | 0.492 | −0.611 | −0.326 | −0.903 | −0.009 | 1.08 | 0.011 | −1.238 |
| MSH3 | −1.49 | −0.922 | −1.174 | −0.893 | −1.102 | −0.389 | −0.346 | −0.698 | −0.063 | 0.097 | 0.136 | −0.454 | 0.23 | 0.22 | −0.42 |
| MSMB | −0.425 | −0.685 | −0.64 | −0.698 | −0.316 | −0.352 | −0.837 | 0.012 | −0.845 | −0.845 | −0.64 | −0.468 | −1.037 | −0.193 | −0.677 |
| MTA1 | −0.4505 | −0.8685 | −0.0805 | −0.3955 | 0.4535 | −1.2945 | −0.1555 | −0.6685 | −1.2585 | −0.7195 | −0.4955 | −0.5095 | 0.1055 | −0.2245 | −0.2175 |
| MTA3 | −0.249 | 1.288 | −0.114 | 0.212 | 0.523 | 0.479 | −0.907 | 0.275 | 0.134 | 0.107 | −0.347 | 0.061 | −0.145 | 0.173 | 0.404 |
| MTAC201 | −0.001 | 0.471 | 0.822 | 0.302 | −0.329 | −0.097 | 0.874 | 0.031 | 0.327 | −0.33 | −0.354 | 0.656 | −0.417 | 0.115 | 0.183 |
| MTCH1 | 1.1195 | 0.3805 | 0.9125 | 0.3485 | 1.4495 | 1.5335 | 2.0155 | 0.3425 | −0.1235 | 0.0875 | −0.8505 | −0.4585 | −1.3005 | −1.0455 | −0.3035 |
| MTHFD1 | −3.9905 | −3.0525 | −1.9825 | −0.5115 | −2.6585 | −1.5015 | −1.5505 | −2.6915 | −1.4365 | −2.5045 | −0.5815 | −1.1685 | −2.0855 | −0.6895 | −1.2275 |
| MTMR2 | −1.7465 | −1.2125 | −0.5745 | −0.5415 | −0.3285 | 1.3995 | −0.4485 | −0.3785 | 0.1215 | 0.0695 | 0.1295 | 0.0675 | 0.6125 | −0.0725 | 0.1875 |
| MUC1 | −1.062 | 0.132 | −1.214 | −0.514 | −0.407 | −0.353 | −1.342 | 0.062 | 0.365 | 0.558 | 0.825 | −0.386 | −0.227 | −0.06 | 0.489 |
| MUC20 | 0.902 | −0.952 | −0.907 | 0.262 | −0.014 | −0.103 | −0.982 | 0.835 | 0.246 | 0.398 | 0.155 | 0.376 | −0.635 | 0.164 | 0.405 |
| MUM1 | 0.122 | −0.492 | −2.257 | −1.321 | −3.023 | −4.316 | −0.837 | −0.926 | −1.098 | −2.3 | −0.444 | −2.116 | 0.18 | 0.019 | −2.937 |
| MXI1 | 0.608 | 2.028 | 2.822 | 2.512 | 1.568 | 1.412 | 1.568 | −0.606 | −0.706 | −0.247 | 1.032 | −0.432 | 0.108 | −0.826 | −0.182 |
| MYB | 0.761 | 0.775 | −0.028 | 0.92 | 1 | 0.969 | 2.195 | 0.537 | −1.195 | −1.814 | −0.11 | −0.023 | −1.084 | −0.699 | −1.019 |
| MYBL1 | 0.377 | 1.03 | 0.142 | 2.002 | 0.118 | 0.239 | 1.388 | 0.816 | −0.167 | −0.112 | −0.837 | −0.266 | −0.703 | 0.037 | −0.719 |
| MYBL2 | 0.087 | −1.206 | 0.176 | −0.139 | −0.993 | −0.209 | −0.543 | −0.886 | −1.678 | −1.489 | −1.269 | −1.852 | −1.105 | −1.147 | −1.321 |
| MYO10 | −0.5375 | −0.1005 | 0.3295 | 0.2425 | 0.7355 | 0.1235 | 0.2095 | 0.6035 | −0.3776 | −0.0725 | −0.3285 | −1.0275 | −0.5315 | −0.2175 | 0.0255 |
| MYO6 | 1.2876 | −1.0275 | −0.9205 | −1.6915 | 2.4885 | −2.8795 | −1.5095 | 1.0135 | −0.9715 | −1.4745 | 0.2835 | 0.9345 | 0.0325 | 0.7595 | −0.2315 |
| MYST3 | 0.0375 | −0.2095 | −0.0165 | −0.1205 | 0.1125 | −0.1455 | −0.2245 | −0.2885 | 0.2765 | 0.0885 | −0.0855 | −0.0995 | 0.6395 | 0.2335 | 0.2365 |
| NALP2 | 0.2485 | 0.3435 | 0.4055 | −0.2625 | −0.7895 | −0.0105 | 0.0435 | −0.1025 | −0.6825 | −1.3505 | −0.0805 | −0.1355 | −0.5095 | −0.2625 | −0.9715 |
| NALP6 | −0.5855 | 0.6015 | −0.4075 | −0.1155 | 0.2295 | 1.0585 | 0.8805 | 1.1855 | −0.2935 | 0.1195 | 0.1285 | −0.3155 | −0.9955 | −0.1095 | −0.2795 |
| NANS | −0.819 | −0.418 | −0.35 | −0.499 | −0.206 | −0.462 | −0.527 | −1.125 | −0.357 | −0.302 | −0.35 | −0.498 | −0.404 | −0.417 | −0.718 |
| NARF | −0.347 | −0.052 | −0.706 | 1.938 | −0.76 | −0.07 | 2.053 | 0.512 | 0.09 | 0.232 | 0.23 | 1.274 | 1.607 | 1.671 | 1.486 |
| NAT2 | −0.7235 | −0.1845 | −0.4885 | −0.0715 | −0.9215 | −0.1725 | 0.1125 | 0.6485 | −0.9105 | −0.3085 | −0.1825 | −0.7155 | −0.6035 | 0.1325 | −0.8915 |
| NAV2 | −0.537 | 0.14 | −0.193 | −0.182 | 0.127 | −0.163 | −0.02 | −1.037 | 0.488 | 0.321 | −0.75 | −0.003 | −0.338 | −0.721 | 0.012 |
| NDE1 | −2.4955 | −1.2215 | −1.0545 | 0.8895 | 0.4205 | −0.0945 | −1.1895 | −0.1005 | 1.3375 | 2.1115 | 1.3915 | 1.4435 | 2.0175 | 2.6445 | 2.8825 |
| NDP52 | −0.11 | 0.168 | 0.237 | 0.001 | −0.188 | −0.213 | −0.001 | −0.989 | −0.62 | −0.999 | −0.135 | 0.282 | −0.899 | −0.683 | −0.583 |
| NDRG2 | −0.815 | −0.426 | 0.151 | −0.298 | −0.053 | −0.622 | 0.707 | −0.864 | −1.143 | −1.574 | −0.793 | 0.399 | −0.002 | −0.405 | −0.248 |
| N0UFA7 | −0.6075 | 0.0105 | −0.4936 | −0.3535 | −0.1316 | −0.7355 | −1.1025 | −0.6215 | −0.2505 | −0.4095 | 0.1895 | −0.0305 | −0.5245 | −0.4325 | −0.2925 |
| NDUFB1 | 0.284 | −0.662 | 0.122 | −0.403 | 0.345 | 0.318 | 0.359 | −0.134 | −0.273 | −0.603 | −0.17 | 0.047 | −0.295 | −0.354 | −0.48 |
| NDUFB10 | −0.049 | 0.11 | 0.467 | 0.324 | 0.609 | 1.157 | 0.473 | −0.389 | −0.338 | −0.454 | −0.545 | 0.894 | −0.838 | −0.452 | 0.507 |
| NDUFB2 | 0.6585 | 0.2845 | 1.1635 | 0.5465 | 0.1135 | −0.0095 | 0.5175 | −1.0585 | −0.3465 | −0.5835 | −0.3765 | 0.5855 | −0.9325 | −0.3915 | −0.4895 |
| NDUFB5 | −0.2415 | −0.1785 | −0.1315 | −0.0055 | 0.1615 | 1.1025 | 0.7825 | −0.7365 | −0.3286 | −0.6825 | −0.3285 | −0.2505 | −0.8165 | −0.4505 | −0.4875 |
| NDUFB6 | −0.8426 | 0.0625 | −0.3975 | −0.1755 | 1.5515 | 0.2395 | −0.8595 | −1.0625 | −0.6305 | −0.9545 | −0.5915 | −0.2865 | −1.0025 | −0.4615 | −0.7815 |
| NDUFC2 | 1.4476 | 1.2545 | 1.3045 | 0.0925 | 0.8105 | 0.7805 | 2.3635 | 0.4335 | −0.9255 | −1.5645 | −0.6825 | −1.3615 | −1.4275 | −0.7595 | −1.3075 |
| NEBL | −0.543 | 0.258 | 0.853 | −0.294 | 0.315 | 0.494 | −0.288 | −0.328 | 0.299 | 0.039 | 0.324 | 0.001 | 0.273 | −0.104 | −0.377 |
| NEK2 | −0.261 | −0.735 | 0.652 | 0.004 | 1.085 | −0.58 | 0.351 | −0.397 | −1.229 | 0.1 | −0.677 | −0.582 | −0.618 | −0.602 | −0.135 |
| NEK6 | −0.9836 | −0.9145 | −0.7075 | −0.6505 | −0.9695 | −0.1765 | 0.6035 | −0.1455 | −0.6985 | 0.2235 | 0.3645 | 1.1845 | −0.2925 | 0.4235 | 0.2885 |
| NELL2 | 3.0745 | 1.0045 | 2.9895 | 0.4905 | 1.0345 | 0.3005 | 0.6745 | 2.0915 | −0.7185 | −0.0785 | 0.1235 | −0.2855 | −0.5855 | −0.7295 | −0.6355 |
| NEURL | −1.682 | −2.801 | −1.176 | −0.512 | −1.495 | −0.851 | −1.344 | −0.278 | 1.07 | 1.228 | 0.227 | −0.882 | −0.59 | −0.403 | 0.173 |
| NFE2L3 | 0.2315 | 1.3705 | 0.3425 | 1.3135 | 1.2515 | 0.9915 | 1.0315 | −0.0635 | 0.8505 | 0.6175 | 0.4265 | 0.2035 | 0.1955 | 1.5245 | 1.3075 |
| NFIA | −0.195 | −0.286 | −0.041 | −0.699 | −0.971 | 1.624 | 0.706 | −0.168 | −0.25 | −0.599 | 0.68 | −0.016 | −0.921 | −0.452 | −0.32 |
| NFIB | −0.4435 | 0.0415 | 0.0465 | −1.0885 | 0.7385 | 1.0635 | 1.3215 | −1.2765 | −0.8625 | −0.4505 | −0.8045 | 1.3095 | −0.5875 | −0.8015 | 0.2415 |
| NINJ1 | 0.0955 | 0.0995 | −0.2615 | −0.6205 | −0.0305 | −0.0215 | −0.5285 | −0.0205 | 0.2555 | −0.1895 | −0.1905 | 0.6345 | −0.0175 | −0.0315 | 0.2016 |
| NIPSNAP [symbol] | 0.0045 | 0.8095 | 1.0385 | 0.1295 | 0.5855 | −0.1025 | 0.1345 | −0.4095 | −0.1915 | −0.3285 | −0.0365 | 0.3995 | −0.6185 | 0.1455 | −0.0855 |
| NKX8-2 | 0.5235 | 0.3545 | 0.7455 | 0.0365 | 0.9105 | 0.1285 | 0.3225 | −0.2135 | −0.3415 | −0.3385 | −0.0105 | 0.4865 | −0.8485 | −0.1145 | −0.2955 |
| NME1 | −1.146 | −2.155 | −1.034 | −1.336 | −1.034 | −1.82 | −1.835 | −1.172 | −0.367 | −0.278 | 0.024 | −0.701 | −0.569 | −0.344 | −0.358 |
| NME2 | −0.5285 | −0.3895 | 1.3885 | 0.1035 | 1.6235 | 0.6005 | −0.9205 | −1.3835 | −1.2715 | −1.8305 | 1.2135 | −1.0085 | −1.3885 | −1.4735 | −2.4835 |
| NME3 | 1.8205 | 1.0905 | 1.6865 | 1.1305 | 1.8405 | 1.6875 | 1.0105 | 0.2015 | 0.0075 | 0.2365 | −0.1065 | −0.6495 | −0.4495 | −0.3385 | −0.7495 |
| NMES1 | 0.285 | 0.898 | 1.778 | 0.282 | 0.269 | 0.502 | 1.414 | 0.332 | −0.006 | −0.121 | −0.043 | 0.338 | 0.429 | −0.281 | −0.345 |
| NMI | 1.3955 | −0.0135 | 0.8035 | 0.3585 | −0.1085 | −0.3295 | −0.6785 | 0.1155 | 0.4375 | 0.0895 | 0.0815 | 0.1045 | 0.0695 | 0.1745 | −0.2195 |
| NOC4 | 1.1085 | 2.8805 | 2.5485 | 0.8995 | 1.8415 | 1.1645 | −0.0945 | 2.3605 | 0.4445 | 0.2385 | −0.0145 | 0.1515 | −0.5295 | −0.4245 | −0.1385 |
| N0LC1 | 0.8585 | 0.5645 | 1.0935 | 0.7645 | 1.5525 | 1.1945 | 0.9265 | −0.6305 | 0.3825 | 0.4315 | −0.5345 | −1.0945 | −0.4005 | −0.0245 | 0.5345 |
| NotFound | −0.2085 | 0.8005 | 0.3475 | −0.0915 | 0.5225 | −0.4415 | 0.1255 | −0.2865 | −0.4095 | 0.0165 | −0.0125 | 0.5845 | 0.4275 | 0.1385 | 0.1525 |
| NotFound | 0.204 | 1.732 | 1.559 | 0.503 | 0.529 | 1.195 | 0.05 | 1.61 | 0.558 | −0.049 | 0.468 | 0.088 | −0.065 | −0.328 | −0.289 |
| NotFound | 0.722 | −3.102 | −0.381 | −1.69 | −1.29 | −0.628 | −1.559 | −1.418 | −0.988 | −0.929 | 0.871 | −0.024 | −0.082 | 0.157 | 0.09 |
| NotFound | 0.129 | 0.79 | 1.784 | 0.92 | 2.597 | −0.003 | [symbol] 207 | 1.022 | −0.896 | 0.31 | −0.072 | −0.011 | −0.074 | −0.914 | −0.842 |
| NotFound | 1.7195 | 1.8575 | 2.8695 | 2.0755 | 1.9995 | 2.1745 | 1.1465 | 1.4435 | −1.4325 | −0.1355 | −0.0316 | −0.3885 | −1.2355 | −0.5235 | −0.5785 |
| NotFound | −0.63 | 2.989 | −0.565 | 1.44 | 2.779 | 2.681 | 2.984 | −0.731 | 1.004 | 1.46 | 1.218 | 2.181 | 1.014 | 1.497 | 0.389 |
| NotFound | −1.7105 | 0.2815 | 0.3775 | −1.0855 | −0.2485 | 0.2635 | 0.1865 | −0.8485 | −1.7235 | −2.4075 | −2.2345 | −1.0095 | −2.1675 | −1.8565 | −2.1765 |
| NotFound | 1.42 | 0.607 | 1.572 | 1.075 | 1.139 | 1.039 | 1.323 | 0.582 | −0.4 | −0.389 | −0.647 | −0.033 | −0.373 | −0.165 | −0.175 |
| NotFound | −0.1535 | 0.2265 | 0.0595 | 0.3515 | −0.2285 | −0.3335 | −0.7725 | 0.7605 | 0.6525 | 0.5045 | 0.9125 | 0.3305 | 0.2505 | 0.8855 | 1.6445 |
| NotFound | 0.4545 | 1.1335 | 1.5795 | 0.7655 | 1.0365 | 1.3175 | 0.4015 | 0.4235 | 2.5175 | 2.2875 | 1.2925 | 0.6395 | −1.9775 | −1.1915 | −0.0335 |
| NotFound | 0.7505 | −0.2355 | −0.4015 | −0.3195 | −0.0205 | 0.5875 | 0.2905 | 1.0485 | −0.5895 | −0.2365 | 0.5655 | −0.0645 | −0.9465 | −0.2285 | −0.3205 |
| NotFound | 0.0955 | 0.0385 | −0.1745 | 0.0805 | 0.4835 | −0.0145 | 0.0925 | 0.3125 | 0.9875 | 0.8195 | 0.8855 | −0.1325 | 1.1625 | −0.0275 | −0.0095 |
| NotFound | −2.535 | −1.081 | 0.587 | −0.103 | −0.506 | −0.525 | −0.44 | 0.503 | −0.447 | −0.209 | 0.267 | 0.914 | 1.81 | 1.109 | −0.229 |
| NotFound | −0.035 | −0.165 | −0.611 | −0.659 | −0.108 | 0.412 | −0.594 | 0.113 | −0.199 | −0.191 | −0.07 | −0.181 | −0.945 | 0.218 | −0.002 |
| NotFound | −0.809 | −0.154 | −2.624 | −1.43 | −1.241 | −1.011 | −2.386 | −0.404 | −0.483 | −1.054 | −0.262 | −1.018 | −1.242 | −0.335 | −0.855 |
| NotFound | −0.6825 | −1.0715 | −0.1705 | −0.8855 | −0.8095 | −0.9055 | −1.9635 | −0.8305 | −0.1105 | −0.1405 | 0.8745 | −0.1275 | −0.4535 | 0.0985 | −0.3985 |
| NotFound | −0.582 | −0.215 | −0.691 | 0.002 | −0.517 | 0.513 | −0.93 | 0.2 | −0.568 | 0.393 | 0.829 | −1.538 | 0.88 | −0.125 | 0.207 |
| NotFound | 1.219 | −0.068 | 0.17 | −0.024 | 0.801 | −0.404 | 0.355 | 0.814 | 0.077 | 0.641 | 0.499 | 0.749 | 0.378 | 0.619 | 0.583 |
| NotFound | 0.6175 | 0.0205 | 0.0285 | 0.0005 | 0.4125 | 0.4905 | −0.3715 | −0.5825 | −0.3795 | −0.7005 | −0.5855 | −0.4965 | −0.8825 | −0.7385 | −0.8145 |
| NotFound | −1.8245 | −1.9425 | 0.4985 | −0.8345 | −0.3575 | −2.7835 | 0.0475 | −1.7735 | 2.8175 | 0.6475 | 1.0845 | 1.1035 | 1.7765 | 1.2155 | 2.1385 |
| NotFound | 0.145 | −0.854 | −1.086 | −0.4 | 0.789 | −0.475 | −1.148 | 0.368 | 0.143 | 0.371 | 0.553 | 0.707 | 0.236 | −0.081 | 0.451 |
| NotFound | 0.1825 | 0.1435 | 0.5895 | 0.3015 | 1.0225 | 2.1595 | 2.1875 | 1.8245 | 0.8125 | 0.1505 | −0.6785 | 1.6485 | −0.2365 | 0.3855 | 1.0795 |
| NotFound | −1.84 | −0.882 | −0.919 | −0.94 | −0.735 | −0.822 | −1.543 | −2.889 | −1.569 | −1.655 | −0.626 | −1.367 | −0.039 | −1.058 | −1.608 |
| NotFound | −1.254 | 0.0658 | 0.824 | −0.432 | −0.171 | −0.47 | 0.948 | −0.557 | −1.135 | −1.724 | −1.187 | −0.355 | −0.784 | −0.328 | −0.785 |
| NotFound | 2.1545 | 1.0045 | −0.3885 | 0.3825 | 1.1825 | 0.9955 | 0.6805 | 0.4475 | −0.7895 | −0.3545 | −0.1185 | 0.2975 | −0.3305 | −0.8725 | −0.4135 |
| NotFound | 1.499 | 0.035 | 0.848 | 0.237 | 0.605 | 1.371 | 0.534 | 0.849 | −0.58 | −1.271 | −0.774 | −0.709 | −0.881 | −0.543 | −0.342 |
| NotFound | −1.3995 | 0.3175 | 0.4495 | −0.5245 | −0.5765 | −0.1885 | 0.8276 | −0.9105 | −1.2455 | −1.4365 | −1.4985 | −0.9195 | −1.0795 | −0.8765 | −1.0035 |
| NotFound | −0.9 | −0.646 | 2.757 | 3.869 | −0.382 | 1.033 | 1.489 | 1.316 | 0.175 | −0.434 | −0.108 | 0.232 | −0.42 | 0.072 | 0.198 |
| NotFound | −0.032 | −0.319 | −0.009 | −0.212 | −0.084 | −1.887 | −0.879 | −0.277 | −0.182 | 0.331 | 0.074 | 1.26 | −1.583 | 0.295 | 0.212 |
| NotFound | 0.8965 | 3.4665 | 2.5575 | 1.2455 | 1.7135 | 1.3485 | 0.3055 | 2.7915 | −0.1215 | 0.0875 | −0.5545 | 0.8375 | −0.1795 | −0.2645 | 0.1575 |
| NotFound | −1.6285 | −0.0265 | 0.6985 | −0.4475 | −0.2325 | 0.5815 | 1.0715 | −0.2925 | −1.2265 | −1.2965 | −0.3005 | −0.0685 | −0.3725 | −0.2695 | −0.5125 |
| NotFound | −2.4855 | 0.8185 | 0.1886 | −0.8225 | −0.7335 | 0.1745 | 0.5845 | −0.1885 | −0.5185 | −0.9375 | −0.7665 | −0.4055 | −0.3235 | −0.3675 | −0.2545 |
| NotFound | −0.6835 | 0.8525 | 0.7935 | −0.3055 | −0.1985 | 0.1455 | 0.8195 | −0.4465 | −1.5285 | −1.8885 | −1.5315 | −0.1855 | −0.3386 | −0.4005 | −0.6595 |
| NotFound | 1.5285 | 0.3475 | −0.8075 | −0.2255 | −0.9075 | 0.7895 | −2.0015 | −0.0085 | −1.1755 | −1.7345 | −1.2745 | 0.1345 | −0.7815 | −0.8195 | −1.3495 |
| NotFound | −1.163 | −0.161 | 0.157 | −0.884 | 0.098 | −0.15 | 0.845 | 0.008 | −0.99 | −0.919 | −1.183 | −0.087 | 0.082 | −0.393 | −0.866 |
| NotFound | −2.8055 | 0.5955 | −0.0805 | −0.3385 | 0.2535 | 0.6135 | −0.0975 | −1.8505 | −1.4445 | −2.6055 | −1.8605 | −0.8835 | −1.8015 | −0.7895 | −1.4685 |
| NotFound | 0.4895 | 0.4985 | 0.3275 | 0.4455 | −0.1915 | 0.2705 | 0.6885 | −0.2745 | 0.0165 | −0.2205 | 0.1485 | 0.3475 | −0.2705 | 0.0155 | −0.1915 |
| NotFound | −1.0005 | −0.0035 | 0.9445 | −0.7685 | −0.4225 | −0.0145 | 1.2175 | −0.9625 | −1.5245 | −1.8015 | −0.8995 | −0.1315 | −0.4005 | −0.4435 | −0.4835 |
| NotFound | −1.5995 | −0.6405 | −1.9835 | −0.7865 | −0.4895 | 0.0745 | −0.9345 | −2.2045 | −0.2985 | −0.7995 | −0.2186 | −0.5925 | −1.0205 | −0.8425 | −0.8525 |
| NotFound | 5.395 | 1.894 | 4.298 | 1.237 | 1.314 | 0.307 | 4.241 | 6.821 | −0.202 | 0.81 | 1.699 | 1.057 | 0.521 | 0.204 | 0.074 |
| NotFound | −0.458 | −0.026 | −0.241 | −0.38 | −0.151 | 0.189 | 0.058 | −0.389 | −0.967 | −0.753 | −0.51 | 0.158 | 0.587 | 0.028 | −0.422 |
| NotFound | 0.201 | 3.197 | 1.896 | −0.103 | 1.308 | 1.784 | 0.548 | 2.35 | 0.781 | 1.044 | 0.309 | 0.828 | 0.85 | −0.107 | −0.069 |
| NotFound | −0.8705 | 0.3235 | 0.4245 | −0.5325 | −0.3315 | −0.1075 | 0.9565 | −0.3615 | −1.2745 | −1.3315 | −1.5745 | −0.4735 | −0.7535 | −0.6025 | −0.7445 |
| NotFound | 0.7185 | 0.0005 | 0.5135 | 1.2535 | 2.4505 | 1.4715 | 1.5365 | −0.1805 | −1.0435 | −1.8555 | −1.9455 | −2.2725 | −1.8055 | −1.1315 | −1.8005 |
| NotFound | −0.289 | 2.133 | 0.729 | −0.113 | −0.199 | 0.215 | −0.217 | 0.841 | 0.829 | 0.308 | 0.327 | 0.048 | 1.281 | 0.248 | 0.314 |
| NotFound | 0.0715 | −0.0705 | −0.0045 | 0.1975 | −0.0885 | −0.0885 | 0.0975 | −0.0915 | −0.5985 | −0.1355 | 0.0875 | 0.3255 | 1.6895 | 0.1105 | −0.1835 |
| NotFound | 0.5205 | −0.2085 | 0.6895 | −0.0485 | −0.3535 | 0.2115 | −0.1895 | 0.4075 | 0.2815 | 1.8965 | 0.3515 | −0.1155 | −0.2615 | −0.2065 | −0.0815 |
| NotFound | 1.5695 | −0.8045 | −0.8275 | −0.5895 | −0.0535 | 0.0905 | 1.0655 | 0.1625 | −0.2225 | −0.0465 | −0.4515 | −0.1145 | −0.5125 | 0.1545 | 0.3845 |
| NotFound | 0.7065 | −0.2505 | 0.9135 | −0.2425 | 0.0155 | −0.2185 | −0.7195 | 0.0985 | −0.9735 | −1.4495 | −0.2255 | 0.1205 | −1.0725 | −0.4625 | −0.3115 |
| NotFound | 0.521 | −0.142 | −0.643 | −0.631 | −0.828 | −0.161 | −0.539 | 0.626 | −0.138 | 0.278 | 0.144 | 0.559 | −0.852 | 0.261 | 0.378 |
| NotFound | −2.096 | −0.943 | −1.437 | −0.784 | −1.227 | −0.948 | −1.758 | −0.087 | −0.881 | −1.458 | 0.038 | −0.845 | −0.881 | −0.086 | −0.834 |
| NotFound | 0.9595 | −2.6535 | −0.2885 | −1.5965 | −1.3015 | −0.4516 | −1.4816 | −0.8965 | −0.7435 | −0.7115 | −0.8875 | −0.2385 | −0.2895 | 0.0505 | −0.1825 |
| NotFound | 0.1995 | −0.4895 | −1.7045 | −0.7215 | −0.7695 | −0.7225 | −1.1815 | −0.5835 | −0.3605 | −0.4785 | −0.1885 | −0.0235 | −0.0665 | 0.1615 | −0.0305 |
| NotFound | −3.3435 | −1.4595 | −1.2805 | −1.1595 | 0.8905 | −0.2115 | −1.2835 | −0.9715 | −0.8035 | −0.7905 | −0.1945 | −0.3045 | −0.4875 | −0.3165 | −0.8585 |
| NotFound | 0.3905 | 1.6015 | 0.8475 | −0.4745 | 0.1055 | 0.5055 | 0.3975 | −0.4875 | −0.3235 | 0.4955 | −0.7925 | −1.2855 | −0.5415 | 0.1165 | 0.1345 |
| NotFound | −0.397 | −1.265 | 0.111 | 1.17 | 1.366 | 1.174 | −0.569 | 1.337 | −0.088 | 0.215 | 0.733 | 1.296 | 0.832 | 1.491 | 1.36 |
| NotFound | 0.01 | −0.83 | −0.97 | 1.162 | 0.29 | −0.696 | −0.958 | −1.04 | 1.732 | 1.502 | 0.949 | 1.21 | 0.184 | 0.47 | 1.909 |
| NotFound | −0.2875 | 1.5605 | 0.6225 | 0.0575 | 0.1995 | 0.1275 | −0.0865 | 1.5205 | −0.1985 | −0.1895 | 0.1785 | 0.1645 | 0.5365 | −0.1325 | −0.1405 |
| NotFound | −1.557 | −1.373 | −2.076 | −0.537 | −1.002 | −0.033 | −1.42 | 0.508 | 1.943 | 1.038 | 0.187 | −0.04 | 0.414 | −0.488 | 0.995 |
| NotFound | −0.975 | 0.532 | −0.248 | 0.067 | 0.163 | 0.022 | 1.385 | 0.009 | 0.329 | 0.589 | −0.112 | −0.061 | −0.39 | 0.469 | 0.116 |
| NotFound | −0.4995 | −0.2855 | −0.3945 | −0.5495 | −0.0745 | 0.2015 | 0.2985 | −0.8255 | 0.0895 | −0.6965 | 0.0025 | −0.2555 | −0.9005 | −0.0505 | −0.9455 |
| NotFound | 0.135 | 0.018 | −0.033 | −0.308 | 0.006 | −0.071 | 0.143 | −0.006 | −0.072 | −0.268 | −0.254 | −0.108 | 0.129 | −0.299 | 0.007 |
| NotFound | −2.801 | 1.146 | 0.116 | −1.035 | −0.853 | 0.392 | 0.794 | 0.073 | −1.324 | −1.857 | −1.431 | −0.78 | −0.656 | −1.142 | −0.781 |
| NotFound | −0.9595 | 0.0805 | 0.3885 | −0.1865 | 0.6985 | −1.1515 | −0.1495 | −0.6205 | −1.1385 | −1.2125 | −0.3525 | −0.7555 | −0.5185 | −0.0125 | −0.9085 |
| NotFound | −0.668 | −0.344 | 0.163 | −0.86 | −0.058 | −0.535 | −0.458 | 0.125 | −0.129 | −0.442 | −0.18 | −0.824 | −1.303 | −0.087 | −0.287 |
| NotFound | −0.0915 | 1.0305 | 0.4795 | 0.0015 | −0.1385 | 1.6245 | −0.2375 | 0.4685 | −0.0585 | −0.1135 | 0.5075 | −0.2385 | 0.2815 | 0.1065 | −0.2045 |
| NotFound | −0.3845 | 0.9835 | 0.3045 | 0.2765 | −0.4376 | −0.1205 | 0.1585 | 0.2425 | 0.8665 | 0.8875 | 0.7855 | 0.1515 | 1.3285 | 0.6175 | 0.3175 |
| NotFound | −1.503 | 0.012 | 0.416 | −0.71 | −0.85 | −0.427 | 0.827 | −0.659 | −1.243 | −1.341 | −1.485 | −0.667 | −0.335 | −0.434 | −0.792 |
| NotFound | 1.2565 | 0.6585 | 1.1425 | 0.1685 | 0.3175 | 0.2185 | −0.1645 | 0.0325 | −0.5505 | −0.5555 | −0.2455 | −0.3825 | −0.8935 | −0.4185 | −0.5535 |
| NotFound | −4.116 | −2.473 | −3.489 | −0.999 | −1.519 | −1.125 | −1.622 | −1.915 | 0.179 | 0.103 | 0.18 | −0.44 | 0.585 | 0.168 | 0.005 |
| NotFound | −0.562 | −0.158 | −0.287 | −0.236 | −0.742 | −0.503 | −0.139 | −0.175 | 0.009 | 0.414 | −0.223 | −0.48 | −0.938 | 0.068 | −0.006 |
| NPDC1 | 1.1535 | 0.0135 | 0.0965 | 0.2045 | 0.3146 | −0.3825 | 0.8475 | 0.0805 | 0.1025 | 0.0955 | 0.4785 | 0.2105 | 0.9516 | 0.1035 | 0.3005 |
| NPEPPS | −0.503 | −0.498 | −0.541 | −1.036 | 0.041 | −0.577 | −1.18 | −1.08 | −0.813 | −0.738 | −0.448 | −0.383 | −1.041 | −0.858 | −0.599 |
| NPY | 0.964 | 0.734 | 1.003 | 0.52 | 1.614 | 1.3 | 1.134 | 0.63 | 0.17 | −0.213 | −0.194 | 0.404 | −0.056 | −0.584 | −0.548 |
| NQO3A2 | −0.264 | −1.374 | −0.931 | −0.458 | −0.22 | −1.881 | −1.949 | −0.527 | −0.559 | −0.484 | −0.72 | −1.384 | −2.515 | −0.667 | −1.159 |
| NRAS | −3.314 | −2.518 | −3.425 | −1.169 | −0.991 | −0.19 | −0.113 | −3.611 | 0.528 | 0.388 | 0.76 | −0.102 | 0.24 | −1.165 | 0.928 |
| NRIP1 | 0.0295 | −0.2965 | −0.4455 | −0.7445 | −0.5225 | −1.0565 | −0.4985 | −0.5885 | −0.4575 | −0.3555 | −0.8995 | −2.0405 | −0.9805 | −0.5195 | −0.3145 |
| NTN4 | 1.3625 | 0.8895 | 1.0695 | 0.2495 | 1.2845 | 1.1445 | 0.6895 | −0.2495 | −0.3215 | −1.1415 | −0.1105 | 0.0745 | −0.7525 | −0.7485 | −0.5895 |
| NUCB2 | 0.3475 | 0.0255 | 0.0195 | −0.3085 | 0.0815 | −0.5895 | −0.0045 | −0.7395 | −0.5275 | −0.1365 | −0.0355 | 0.4515 | −0.3775 | 0.0285 | 0.1795 |
| NUDT5 | 0.189 | 0.301 | 0.243 | 0.379 | 0.585 | 1.392 | 0.395 | 0.157 | −1.317 | −0.821 | −0.264 | −0.102 | −0.813 | −0.433 | −0.889 |
| NUDT6 | −0.306 | 0.002 | 1.038 | −0.238 | 0.248 | 0.015 | 0.962 | 0.534 | −0.019 | 0.573 | 0.039 | −1.16 | 0.358 | 0.652 | 0.348 |
| OCIAD2 | 3.9515 | 1.6055 | 1.3685 | 1.5475 | 2.0775 | 2.1035 | 3.4265 | 1.6755 | −0.1205 | −0.3455 | 0.4275 | 0.1825 | −0.4815 | 0.1185 | −0.0985 |
| OFD1 | −0.0865 | −0.4725 | 0.8735 | −0.5615 | 2.0455 | 0.8505 | 1.4845 | −0.2615 | −0.2335 | −0.6535 | 0.5975 | 0.2515 | −0.4605 | −0.1285 | −0.0555 |
| OGFRL1 | −0.693 | 0.995 | −0.502 | −0.314 | −0.352 | −0.329 | −0.732 | −0.043 | 0.84 | 0.469 | 0.408 | −0.148 | 0.381 | 0.001 | 0.239 |
| OPLAH | −0.2795 | 0.4885 | 0.6205 | 0.2395 | 0.1505 | 0.5025 | 1.7045 | 0.9535 | −0.6185 | −0.7255 | 0.0795 | −1.2475 | −0.2785 | −0.5525 | −0.7426 |
| ORMDL3 | −0.617 | 0.445 | −2.209 | −0.398 | 0.151 | 1.292 | −0.892 | −1.39 | 1.135 | 2.608 | −0.263 | −0.238 | −0.142 | −0.501 | 0.499 |
| OSBPL3 | −0.575 | 0.186 | 0.885 | 0.458 | 0.842 | 1.12 | 0.592 | −0.71 | −0.006 | 0.003 | 0.027 | 0.184 | −0.498 | −0.394 | −0.038 |
| OSR2 | 0.0855 | 1.9315 | 1.0835 | 0.2955 | 0.9415 | 0.8126 | 0.6675 | 1.4405 | −0.1585 | −0.1965 | −0.3825 | −0.1315 | 0.5475 | −0.5585 | −0.8955 |
| OSTF1 | −2.589 | −1.704 | −1.3 | −1.28 | −1.349 | −1.045 | −2.252 | −1.046 | −1.213 | −1.557 | 1.899 | −0.585 | −0.383 | −1.522 | −1.859 |
| P2RX1 | −1.5015 | 2.8825 | 1.6555 | 0.2555 | 0.5785 | 0.4275 | 0.1865 | 2.9355 | 1.1445 | 0.6665 | 0.6525 | 0.2455 | −0.1625 | 0.0605 | −0.0595 |
| P2RY2 | −0.054 | 0.04 | 0.112 | 1.204 | 0.297 | 1.821 | 0.343 | −0.638 | −0.074 | 0.407 | 0.062 | 1.864 | −0.803 | 1.535 | 0.89 |
| PACAP | −0.298 | 0.355 | 0.065 | −0.249 | −0.236 | −0.193 | −0.449 | 0.188 | −0.029 | 0.018 | 0.002 | −1.665 | 0.477 | 0.271 | −0.081 |
| PADI2 | 0.9875 | 1.3625 | 1.0505 | 0.5375 | 0.8815 | −0.9845 | 0.4745 | 0.6425 | 0.5385 | −1.2795 | 0.4445 | 0.0565 | −1.4855 | −0.0215 | 0.0955 |
| PAPD5 | −0.028 | −0.749 | −0.792 | −0.096 | −0.168 | −0.494 | 0.322 | 0.266 | 0.108 | −0.606 | 0.361 | −0.083 | 1.008 | 0.064 | −0.15 |
| PAPSS2 | 1.396 | 0.688 | 0.59 | 0.227 | −0.055 | −0.175 | 0.648 | 0.891 | −0.887 | −1.047 | −0.272 | −0.403 | −1.1 | −0.8 | −1.122 |
| PARD8A | −0.147 | 1.157 | 0.939 | −0.172 | 1.306 | 1.062 | 0.747 | −0.671 | −0.85 | −0.869 | −1.061 | −1.317 | −2.267 | −0.995 | −0.901 |
| PARP1 | −0.249 | 1.503 | −0.107 | −0.418 | 0.027 | 0.257 | 0.348 | 0.015 | −0.4 | −0.493 | −0.296 | −0.539 | −0.859 | −0.273 | −0.739 |
| PARP9 | −0.3325 | 0.5755 | −0.2885 | −0.1305 | 0.1265 | 0.1475 | −0.1115 | 0.6885 | 0.1125 | −0.2125 | −0.0405 | −0.0135 | −1.2615 | −0.1625 | −0.2405 |
| PAXIP1L | 1.2805 | −0.3135 | 0.7385 | 0.1425 | 1.1775 | 0.6355 | 1.3125 | −0.6985 | 0.4645 | 0.5275 | −0.3835 | 0.1525 | 0.1695 | 0.1165 | 0.2535 |
| PBX3 | 1.6945 | 0.1095 | −0.2625 | −0.2105 | 1.2045 | −0.7105 | 1.7895 | −1.4435 | 0.1455 | 0.8095 | −0.0085 | −0.0615 | −0.0565 | −0.0735 | 0.0645 |
| PCDHB11 | 1.083 | −0.205 | 2.553 | 1.011 | 0.98 | 1.584 | −1.98 | 0.574 | 0.415 | −0.187 | −0.129 | 0.457 | −1.034 | −0.305 | 0.122 |
| PCDHB16 | 0.383 | −0.881 | 0.433 | 0.325 | 0.002 | 0.591 | −0.352 | −0.32 | −0.518 | −0.002 | −1.1 | −1.935 | −0.591 | 0.158 | −0.256 |
| PCDHB2 | 1.0875 | 1.2115 | 0.8185 | 0.6035 | 0.6825 | −0.3505 | 1.0525 | 1.0785 | −0.3405 | −0.3565 | −0.2835 | 0.5515 | −1.3585 | −0.2845 | −0.7255 |
| PCM1 | 0.4065 | 0.6555 | 1.9195 | 0.7995 | 1.4435 | 1.9825 | 0.6425 | 1.8575 | 0.8395 | 0.5145 | 0.6735 | −0.7605 | 1.1415 | 0.0265 | −0.2485 |
| PCNA | 0.899 | 0.167 | 0.491 | 0.583 | 1.113 | 1.258 | 1.017 | −0.958 | −0.767 | −0.319 | −0.713 | −0.994 | −1.462 | −0.791 | −1.034 |
| PDCD1 | −1.2615 | −1.7585 | −0.7015 | −0.1575 | 0.1795 | −1.5025 | −2.0205 | −0.8995 | 0.6405 | 0.4525 | 1.7935 | −0.5425 | 0.0545 | 0.9615 | 0.6065 |
| PDCD10 | 1.287 | 0.382 | 1.221 | 0.869 | 0.72 | 0.488 | 3.213 | 0.44 | −0.432 | −0.455 | −0.291 | −0.229 | 0.102 | −0.545 | −0.571 |
| PDCD4 | 0.4715 | 0.2876 | 0.4225 | 0.1105 | 0.0635 | −0.4835 | −0.4155 | −0.1155 | −0.1665 | −0.7955 | −0.2325 | 0.1295 | −0.8925 | −0.9725 | −0.7185 |
| PDCD5 | −1.6045 | 0.4555 | 2.2955 | 0.8865 | −0.5805 | 1.2315 | −0.7175 | 0.7455 | 1.8575 | 0.7355 | −0.3785 | 0.3535 | 0.9045 | −0.0855 | 1.0235 |
| PDCD8 | −0.0816 | 0.3815 | 0.1565 | 0.5895 | 0.6835 | 0.9855 | 1.8055 | 0.2325 | 0.3175 | 0.2525 | −0.0925 | 0.4065 | −0.1185 | −0.0245 | 0.1205 |
| PDE4DIP | −0.8875 | −1.5745 | −2.1475 | −1.3735 | −0.8026 | −0.7265 | 1.1575 | −1.4705 | 0.0325 | −0.4055 | −0.1565 | 0.1665 | −0.0235 | −0.2655 | −0.2035 |
| PDHA1 | 3.183 | 2.641 | −0.487 | −0.227 | 0.883 | 2.66 | 3.017 | 0.478 | −0.859 | −0.157 | 1.668 | −0.103 | 0.335 | 0.314 | −0.2 |
| PECI | −1.5115 | −1.0735 | −1.7235 | −0.1035 | −1.2485 | 0.5195 | −1.8125 | −1.7525 | 1.3075 | 1.2505 | 0.0415 | 1.0605 | −0.3475 | 0.0195 | 0.3825 |
| PEG10 | 1.4255 | 0.0595 | −0.1735 | 2.1675 | 0.4905 | −0.0905 | 3.2795 | 0.1295 | −0.2925 | −0.1935 | −0.2575 | 0.1835 | −0.1075 | 0.0965 | −0.2745 |
| PER2 | 0.385 | 1.645 | −0.174 | −0.604 | −0.306 | 0.02 | −0.517 | −0.192 | −0.873 | −0.51 | −0.773 | −1.38 | −1.483 | 0.034 | −0.332 |
| PERP | 0.5855 | −0.5525 | −0.2305 | 0.2895 | −0.5795 | −0.0975 | −0.2455 | −0.8325 | 0.6945 | 1.0195 | 0.4415 | −1.5255 | 0.9685 | 0.4505 | 0.3465 |
| PEX1 | 1.293 | −0.316 | 1.084 | 0.813 | 0.907 | 0.161 | 0.405 | 0.155 | 0.131 | −0.474 | −0.241 | 0.083 | −0.947 | −0.834 | −0.486 |
| PFAAP5 | −1.1395 | −0.5075 | −0.8256 | −0.4505 | −0.1825 | −0.0135 | 0.3895 | −0.0955 | −0.0815 | 0.6905 | 0.2755 | 0.8805 | 0.4865 | 0.3205 | 0.7175 |
| PFDN2 | 0.273 | 0.212 | −0.181 | −0.365 | −0.042 | −0.575 | −0.721 | −1.196 | 0.299 | 0.518 | −0.204 | −0.021 | −0.503 | 0.135 | 0.017 |
| PGM1 | 1.0075 | 0.0345 | 0.3765 | 0.1185 | 0.5105 | 0.0645 | 0.0915 | 0.0355 | −0.1975 | −0.1105 | −0.3455 | −1.6305 | −0.6005 | −0.1325 | −0.1835 |
| PGR1 | 0.5245 | 1.4395 | 1.7415 | 1.0155 | −0.4895 | 0.1595 | 0.4475 | 1.9845 | 0.0215 | 0.1835 | −0.0845 | 0.8815 | 0.0245 | 0.7885 | 1.0425 |
| PHAX | 0.409 | −0.48 | −0.722 | −0.741 | 0.972 | 0.941 | 0.074 | −1.059 | −0.681 | −1.215 | −1.208 | −0.284 | −1.615 | −1.192 | −0.018 |
| PHGDH | −1.2235 | −1.5895 | −2.5465 | −1.3875 | −1.9755 | −1.4375 | −2.8465 | −1.7505 | −0.2805 | −1.1745 | 0.2875 | −0.5815 | −0.0055 | −0.4845 | −1.8155 |
| PHYH | −0.7995 | −0.2855 | −0.1805 | −0.4705 | −0.2555 | −0.2265 | −0.2985 | −0.6355 | 0.0545 | −0.1935 | −0.1335 | −0.3715 | −0.9435 | −0.5765 | −0.8476 |
| PI85PA | −0.4595 | 1.9425 | 1.4785 | 0.8515 | 1.5525 | 0.6295 | 0.1755 | 2.5945 | 1.3525 | 0.3805 | 1.5085 | −0.0595 | −0.0015 | −0.8045 | −0.2195 |
| PIGT | −1.075 | −0.749 | −0.388 | −0.549 | −0.419 | −0.664 | −0.753 | −0.317 | 0.24 | −0.107 | 0.063 | 0.246 | −0.37 | 0.001 | 0.185 |
| PIM2 | −0.3185 | 0.8355 | 0.3165 | −0.2275 | 0.4325 | 0.1085 | −0.0975 | −0.1365 | 0.6855 | 0.7415 | 0.3475 | 0.1805 | −0.5625 | 0.1635 | 0.8585 |
| PISD | −1.562 | −0.211 | −0.901 | 0.087 | 0.152 | −0.136 | 1.834 | −1.36 | −1.904 | −1.698 | −0.954 | 0.446 | −2.375 | 0.015 | −1.579 |
| PITPNC1 | 2.6715 | 1.0925 | 0.0395 | 0.2335 | 0.2785 | −0.6815 | 0.7135 | −0.7235 | −0.5305 | −1.4885 | 0.2775 | −0.0015 | −0.7285 | −0.8885 | −1.0175 |
| PKIB | −1.687 | −0.071 | −1.09 | −0.539 | −0.289 | 0.192 | −1.323 | −0.513 | −0.295 | −0.313 | −0.058 | 0.384 | 0.289 | −0.284 | −0.05 |
| PKMYT1 | 0.068 | 1.436 | 3.172 | 1.739 | 3.298 | −1.222 | 2.067 | 0.329 | 1.085 | 0.307 | 1.993 | 1.764 | 0.843 | −0.037 | 0.127 |
| PLA2G4B | −0.8205 | −0.0825 | −1.4555 | −0.2715 | −0.1265 | 0.9885 | 0.4425 | 0.0286 | 0.3375 | 0.6125 | 0.4765 | −0.7195 | 1.1845 | 0.9005 | 0.9135 |
| PLAC8 | −1.31 | −0.18 | 0.884 | −0.193 | −0.62 | −0.823 | 0.24 | 0.129 | 0.019 | −0.577 | 0.058 | 0.68 | 0.24 | 0.501 | 0.47 |
| PLCB1 | 0.6276 | 0.8535 | 0.4835 | 0.1255 | 0.5435 | 0.0835 | 0.0215 | 0.1215 | −1.0005 | −0.9375 | −0.9535 | 0.3355 | −1.1085 | −0.6595 | −0.3825 |
| PLCXD1 | 0.175 | 1.149 | 0.767 | 0.968 | 1.038 | 1.826 | 0.258 | 0.194 | −0.062 | −0.574 | −0.306 | 0.492 | −0.781 | 0.535 | 0.536 |
| PLEK2 | 0.8455 | 0.4245 | 0.7905 | 0.8855 | 0.2655 | 0.9295 | 1.5925 | −0.1395 | −0.2835 | −0.1755 | −0.4525 | −0.8365 | −1.3605 | −0.4855 | −0.2445 |
| PLO0 | 0.3395 | −0.2795 | −0.0825 | 0.3595 | 0.9945 | 1.2905 | 0.8015 | −0.2155 | 0.3065 | −0.0415 | 1.7105 | 0.5615 | −0.0255 | −0.4025 | −0.1045 |
| PNAS-4 | 2.0075 | −0.6355 | −1.0015 | −0.2205 | −0.0765 | 1.8235 | 2.0145 | 0.0405 | 0.0105 | 0.9835 | 0.3275 | 0.0895 | 0.0845 | −0.2765 | 0.3345 |
| PNLIPRP2 | 0.3315 | −0.3555 | −0.0176 | 0.0175 | 0.9125 | 0.0145 | 0.1475 | −0.4795 | −1.0135 | −0.7195 | −0.4545 | −0.3885 | −1.3655 | −0.3995 | −0.9805 |
| PNMA3 | 0.1545 | 0.7825 | 0.3245 | 0.2785 | −0.0525 | 0.5025 | 0.4385 | 0.0845 | 0.0075 | −0.1265 | 0.1555 | 0.4265 | 0.3275 | 0.4165 | 0.1085 |
| POLB | 0.198 | −0.366 | 0.471 | 0.06 | 0.063 | 0.188 | −0.951 | −0.163 | −0.573 | −0.51 | −0.335 | 0.073 | −0.638 | −0.005 | −0.588 |
| POLR2F | 0.6335 | 0.1135 | 0.1195 | −0.0285 | 1.3345 | 0.9995 | 2.0705 | 0.5015 | −0.1585 | 0.2735 | 0.0875 | 0.9535 | 0.5705 | −0.3305 | 0.2635 |
| POLR2H | 0.0595 | 0.0665 | 0.6415 | −0.4205 | 0.0295 | 0.5555 | 2.0335 | −0.3175 | −0.8825 | −0.6105 | −0.4545 | −0.0805 | −1.2015 | −0.5075 | −0.6515 |
| POMC | 1.038 | 0.767 | 0.056 | −0.199 | 0.272 | −0.081 | 0.622 | 0.173 | −0.36 | −0.555 | −0.001 | −0.021 | −0.171 | −0.582 | −0.495 |
| POP4 | −0.845 | −1.167 | −1.906 | −0.555 | −0.429 | 0.841 | −0.064 | 0.699 | 0.051 | −0.138 | 0.048 | −0.19 | 0.617 | −0.697 | −0.42 |
| POP7 | −0.03 | 2.144 | −0.13 | −0.288 | 0.464 | 0.199 | 0.919 | 0.195 | 0.095 | 0.082 | 0.95 | −1.165 | −1.285 | −0.553 | −0.529 |
| PP1057 | 1.4215 | −0.2255 | 0.7575 | 0.3625 | −0.1585 | −0.0995 | −1.2376 | 0.8385 | −0.9465 | −0.5385 | 0.3405 | −0.4375 | −0.3805 | −0.1305 | −0.2455 |
| PPFIA1 | 0.232 | 0.383 | 0.512 | −0.272 | 0.538 | 0 | −0.193 | 0.04 | 0.106 | −0.478 | 0.845 | −0.508 | 0.138 | −0.639 | −0.291 |
| PPFIBP2 | 0.0705 | 1.2215 | 0.1005 | −0.1135 | 0.2875 | −0.4415 | −0.8105 | −0.0955 | −0.5145 | 0.2535 | −0.0875 | −0.2825 | −0.4875 | 0.0885 | −0.3305 |
| PPGB | 0.2525 | 0.1555 | 0.4085 | 0.0695 | −0.0685 | 0.6215 | −0.5565 | 0.8985 | −0.2705 | −1.0975 | 0.3025 | 0.5825 | −0.2825 | −0.3075 | −0.7185 |
| PPHLN1 | 0.5925 | 2.1625 | 0.7675 | 0.5245 | 0.4915 | 0.3335 | −0.3005 | −0.0525 | −0.7505 | −0.9125 | −0.7945 | 0.6275 | −0.6835 | −0.8835 | −0.8135 |
| PPIF | 0.2445 | −0.2165 | −0.0315 | −0.1485 | −0.3405 | −0.2885 | −0.5575 | 0.0415 | −0.1995 | 0.0505 | 0.0905 | −0.4565 | −0.1615 | 0.1335 | 0.1885 |
| PPIL1 | −0.2165 | −0.6965 | 0.8685 | −0.1915 | −0.3585 | 0.0395 | 0.3075 | −0.9125 | −0.3785 | −0.8125 | 0.3855 | −0.3035 | 0.2285 | 0.2805 | −0.1595 |
| PPOX | −2.002 | −2.013 | −2.453 | −0.655 | −1.285 | −2.07 | −1.475 | −2.127 | −0.68 | 0.035 | −0.833 | −0.131 | 1.234 | −1.114 | 0.561 |
| PPP1R16 [symbol] | −0.054 | −0.249 | −0.38 | −0.664 | −0.001 | 0.478 | −0.462 | −0.131 | −0.18 | −0.115 | −0.055 | −0.105 | −0.774 | 0.451 | −0.077 |
| PPP1R3C | −1.432 | −0.105 | −0.68 | −0.805 | −0.042 | −0.849 | −0.006 | −1.75 | 0.189 | −0.073 | −0.682 | −0.989 | 0.199 | −0.688 | 0.167 |
| PPP2R2D | 0.7345 | 0.2876 | 3.7995 | −0.0385 | 1.7775 | 2.9635 | 0.5265 | 3.9465 | 1.2545 | −0.1635 | 0.2085 | −0.0195 | 0.3465 | −0.2765 | −0.1785 |
| PPP2R5A | −0.032 | 1.155 | 0.886 | 0.411 | −0.117 | 0.668 | 2.538 | 0.176 | −0.337 | −0.508 | 0.31 | −0.037 | −0.053 | −0.109 | −0.663 |
| PRAME | 1.6285 | 0.9785 | 1.7195 | 0.9405 | 0.8995 | 0.2035 | 0.8075 | 0.2195 | −0.5485 | −0.3085 | −0.4495 | 0.5705 | −0.7605 | −0.0595 | −0.1025 |
| PRC1 | −0.477 | 0.403 | 0.022 | 0.238 | 0.632 | −0.627 | 0.07 | 0.577 | 0.042 | 0.321 | −0.42 | −0.1 | 0.058 | −0.124 | −0.012 |
| PRIM2A | −1.0426 | 1.0486 | 3.0655 | 2.0535 | 2.3546 | −0.1095 | 1.0295 | 0.7715 | −0.0525 | −0.3985 | 1.4455 | −0.1376 | −0.2735 | 0.2565 | 0.0205 |
| PRKACB | 0.893 | 0.747 | −0.323 | −0.37 | 0.255 | 1.137 | 0.583 | −0.147 | −0.242 | 0.157 | −0.434 | −1.717 | −1.341 | −0.262 | −0.407 |
| PRKCB1 | 0.226 | 0.113 | 0.463 | 0.128 | 0.725 | 0.907 | 1.526 | −0.003 | 0.198 | 0.26 | −0.215 | −0.834 | 0.235 | 0.177 | −0.114 |
| PRKCI | 0.000 | 0.357 | 0.131 | −0.272 | 0.558 | 1.211 | 0.015 | 2.39 | 0.288 | −0.013 | 0.102 | −1.089 | −1.568 | 0.096 | −0.102 |
| PRKRIR | 0.0635 | 0.2585 | 1.2855 | 0.8685 | 1.4495 | 0.7755 | 1.0925 | 1.1685 | −0.3865 | −0.2535 | −0.2565 | 0.1985 | −0.2555 | 0.3625 | 0.6476 |
| PRKWNK1 | 1.0185 | 1.0295 | 1.5865 | 0.6445 | 1.7275 | 0.3045 | 0.6395 | 0.8025 | 1.1705 | 1.5355 | 0.1835 | 1.4745 | −0.4055 | 0.1505 | 0.6035 |
| PRKX | −0.092 | 4.009 | 2.332 | 0.471 | 0.17 | 1.214 | 0.094 | 3.22 | 1.07 | 0.42 | 0.701 | −0.076 | 0.472 | −0.231 | −0.1 |
| PRNP | 0.9885 | 0.2275 | 0.1815 | 0.1575 | 1.1245 | 0.4876 | 0.0795 | −1.3565 | −0.2615 | −0.0935 | 0.3205 | −0.9595 | −1.2375 | −0.0085 | −0.0705 |
| PROL5 | −0.2845 | 0.2725 | −0.3875 | −0.1785 | −0.1825 | 0.0035 | 0.6245 | 0.9985 | −0.0855 | 0.2285 | −0.0575 | 0.4385 | −0.1975 | 0.6105 | 0.1295 |
| PRPF18 | 4.0745 | 0.4315 | 4.7395 | 3.3305 | 0.4935 | 3.2985 | 1.3285 | 1.0385 | 0.7635 | 1.1325 | 0.6025 | 0.1005 | −0.2785 | 0.4705 | 0.6815 |
| PRPSAP1 | 0.9965 | 0.0035 | −0.6785 | 1.7485 | 0.7665 | 0.3335 | 0.6555 | −0.1576 | −0.8885 | −0.4965 | −0.4935 | 0.5265 | 0.5875 | −0.1365 | 0.0385 |
| PRSS12 | 1.8035 | −1.2055 | −1.1635 | −1.1785 | −0.9735 | −0.0455 | −0.7835 | 0.2985 | −1.1865 | −0.7075 | −0.6975 | −1.0035 | 0.6335 | −0.0645 | −0.7025 |
| PRSS16 | 0.4895 | 0.4235 | 1.2105 | 0.8435 | 0.8805 | 0.0245 | 0.8515 | −0.5545 | −0.3885 | −0.2785 | −0.6195 | 0.2405 | −0.4865 | −0.5075 | −0.3576 |
| PSD3 | 0.8895 | 0.3655 | −0.1555 | −0.3645 | 0.5435 | 1.2025 | 0.5585 | 0.6005 | −0.1535 | −0.4855 | 0.2285 | −0.7425 | −1.1815 | −0.8205 | −0.9115 |
| PSMA3 | −1.068 | −0.03 | −0.035 | −0.374 | 0.067 | 0.179 | −0.584 | −0.36 | 0.14 | 0.102 | 0.222 | −0.198 | −0.134 | 0.479 | −0.136 |
| PSMA7 | −0.241 | 0.288 | 0.237 | 0.213 | 0.714 | −0.155 | 0.584 | −0.899 | −0.734 | −0.295 | −1.361 | −0.322 | −1.094 | −0.608 | −0.811 |
| PSMC5 | 0.8205 | 1.0385 | 0.5435 | 0.7195 | −0.3855 | 0.4755 | −0.7885 | −0.4425 | −0.1415 | −0.3135 | −0.9705 | −1.6025 | −1.2275 | −0.6145 | −0.3855 |
| PSMC6 | 0.602 | 1.228 | 1.107 | 0.514 | 0.429 | 1.14 | 0.603 | −1.371 | 0.148 | 0.05 | −0.767 | −1.211 | −0.486 | −0.547 | −0.773 |
| PSMD12 | −0.6055 | 1.8725 | −0.4265 | −0.4675 | −0.0015 | −0.1305 | −0.7685 | 0.1935 | 0.2815 | 0.3035 | 0.2885 | 0.7185 | −1.0855 | −0.6305 | −0.0535 |
| PSMD12 | −0.1985 | −0.7435 | −0.0185 | 1.8725 | 0.7735 | −1.0875 | 0.8485 | −0.0115 | 0.7275 | −0.0075 | −0.1275 | −1.0355 | −1.9945 | 0.5045 | −0.2095 |
| PSMD3 | 0.2165 | −0.4795 | −0.7825 | −0.4595 | 1.1055 | 0.5836 | 0.8925 | 0.1625 | −0.7885 | −0.5275 | −0.6925 | −0.3765 | −1.2555 | 0.2835 | −0.3995 |
| PSPH | −0.281 | −0.477 | 0.034 | −0.07 | 0.254 | −0.28 | −0.566 | −0.996 | 1.135 | 0.243 | −0.548 | 0.514 | −0.612 | −0.887 | −0.08 |
| PTD012 | 0.007 | −0.231 | −0.803 | 1.337 | 0.081 | 0.042 | −0.785 | 0.456 | 1.669 | 0.87 | −0.065 | 3.073 | 0.276 | 3.248 | 0.342 |
| PTGER3 | −1.331 | −0.492 | −1.173 | −0.907 | −0.35 | 1.178 | −0.353 | −0.875 | −0.994 | −1.03 | −0.474 | −0.881 | −0.88 | −0.948 | −0.599 |
| PTHLH | 0.3085 | 0.3785 | 0.2495 | −0.4625 | 0.5725 | 0.5315 | 0.0675 | 0.4675 | −0.8505 | −0.4076 | −0.6435 | −0.9375 | −0.9865 | −0.0255 | 0.0085 |
| PTK6 | 0.178 | 0.923 | 1.34 | 0.585 | 1.334 | 0.441 | 0.528 | 1.457 | −0.054 | 0.181 | 0.229 | −0.519 | −0.101 | −0.241 | −0.009 |
| PTPN4 | 0.43 | −0.994 | −0.07 | 1.155 | 0.439 | 0.238 | 0.589 | −0.419 | −0.168 | −1.363 | −0.369 | 0.138 | −1.632 | −0.668 | −0.259 |
| PTPN7 | 0.257 | 0.422 | −0.563 | 0.009 | 0.194 | −0.942 | 0.051 | 0.211 | 0.157 | −0.348 | −0.016 | 0.219 | 1.366 | −0.059 | −0.048 |
| PTPRF | −0.677 | −0.54 | −0.252 | 0.127 | −0.447 | −0.166 | −0.339 | −0.921 | 0.253 | 0.185 | 1 | −0.35 | 0.787 | 0.208 | −0.22 |
| PTPRN2 | 0.2945 | 0.1485 | −0.8915 | 0.5355 | 2.2085 | 2.2625 | 1.7865 | −0.5915 | −0.1845 | −0.5725 | −0.4955 | 0.0525 | −1.0825 | −0.5075 | −1.1115 |
| PTPRT | 1.1075 | 0.8085 | 0.7805 | 0.5435 | 0.6405 | 0.6725 | 1.5935 | 0.9005 | −0.0265 | −0.0075 | 0.8365 | −0.2375 | −0.5745 | 0.0695 | −0.5885 |
| PTS | 0.012 | 0.269 | 0.238 | 0.237 | 0.302 | 1.584 | 1.215 | 1.131 | 0.35 | 0.111 | −0.387 | 0.159 | −1.09 | −0.694 | −0.531 |
| PTTG1 | 0.5965 | 0.0075 | −0.0485 | 0.4195 | 0.2725 | 0.1985 | −0.3995 | 0.1235 | −0.0335 | −0.4925 | 0.7655 | 0.7395 | −1.0585 | 0.0505 | −0.3185 |
| PURB | 1.5445 | 0.0825 | 0.9925 | −0.1876 | −0.5895 | −0.7055 | −1.0755 | 1.2965 | −0.7595 | −0.7315 | 0.8915 | −0.0155 | −0.1795 | −0.4545 | −0.6645 |
| PXMP2 | 0.2035 | 1.2375 | −0.8785 | −0.3975 | −0.0425 | 0.4105 | −0.3715 | −1.0155 | −0.8735 | −1.3155 | −0.1625 | −0.4475 | −1.2315 | −0.3685 | −1.0605 |
| PYCARD | −0.592 | −0.235 | 0.215 | −0.896 | −0.008 | −0.8 | −0.861 | −2.228 | −0.288 | −0.388 | −0.213 | 0.482 | −1.035 | −0.547 | 0.19 |
| PYCR1 | −1.891 | −0.047 | −0.515 | −0.531 | −0.266 | 0.938 | −0.684 | 0.482 | 0.194 | 0.195 | −0.053 | −0.002 | 0.432 | −0.052 | −0.496 |
| QDPR | −0.3515 | −1.1195 | −1.0335 | −0.8135 | −0.2975 | −0.6225 | −1.1105 | −0.0965 | 0.5125 | 0.0285 | 0.1175 | 0.1095 | −0.1285 | 0.6275 | −0.1485 |
| QPRT | 0.085 | −1.771 | −0.508 | 0.003 | −0.776 | −0.003 | −0.414 | −1.636 | −0.271 | −0.565 | −0.457 | 0.106 | −1.238 | −1.309 | −1.553 |
| QSCN9 | 1.6035 | −1.0795 | −0.1445 | −0.8455 | 0.2325 | 0.2795 | −0.0745 | −0.8455 | −1.7345 | −1.7295 | −1.1505 | −1.3385 | −1.8805 | −1.1045 | −2.1285 |
| RAB11FIP | 0.798 | −0.651 | −0.041 | −0.671 | 0.783 | −0.704 | −1.119 | −0.001 | −0.486 | −0.342 | 0.272 | −0.964 | −0.025 | 0.156 | −0.52 |
| RAB11FIP | −0.672 | 1.171 | 1.357 | 1.017 | 0.848 | 0.894 | 0.433 | 0.513 | 0.082 | −0.195 | 0.054 | 0.002 | −0.379 | −0.508 | −0.741 |
| RAB13 | −0.571 | 0.229 | −0.514 | −0.252 | 0.012 | −0.091 | 0.002 | −0.185 | 0.376 | 0.684 | −0.041 | −0.357 | 0.559 | −0.159 | 0.083 |
| RAB27A | −0.057 | 0.304 | −1.19 | −0.316 | 0.124 | 0.245 | −0.68 | −0.474 | 0.029 | 0.603 | 0.255 | 0.195 | −0.252 | −0.001 | 0.261 |
| RAB30 | 0.8225 | 0.3285 | 1.1785 | −0.0855 | 1.0205 | 0.2085 | 0.2135 | 0.3825 | 0.3825 | −0.3385 | 0.3085 | −0.4765 | −0.8765 | −0.4535 | −0.6675 |
| RAB34 | 1.078 | 0.481 | 0.621 | 0.121 | 0.27 | 0.168 | −0.362 | 0.047 | 0.029 | −0.105 | −0.911 | −0.04 | 0.061 | 0.292 | 0.098 |
| RAB35 | −0.026 | 0.074 | −0.182 | −0.095 | 0.094 | 0.134 | −0.194 | 0.222 | 0.251 | −0.145 | 0.01 | 0.936 | 0.197 | 0.195 | 0.211 |
| RAB39B | 0.5325 | −0.3765 | −0.3526 | 0.0005 | −0.1845 | −0.3185 | −1.0965 | 0.5375 | −0.8315 | −0.8555 | 0.1705 | −1.4995 | −0.2385 | 0.0065 | −0.9125 |
| RAB3A | −1.235 | 0.20 | −1.043 | 0.618 | −1.024 | −1.671 | 0.408 | −2.428 | −0.915 | 0.385 | −1.9 | −1.321 | −0.454 | 0.899 | −0.349 |
| RAB3IP | 0.75 | 0.255 | 1.348 | −0.009 | 1.22 | −0.518 | 0.032 | 0.842 | −0.543 | 0.035 | −0.056 | −1.097 | 0.004 | −0.478 | 0.05 |
| RABEP1 | 1.055 | 1.74 | 1.441 | 0.84 | −0.436 | 0.386 | 1.83 | 0.438 | −1.368 | −2.035 | −0.393 | 0.09 | −2.272 | −0.401 | −1.329 |
| RABGAP1 | 0.2455 | −0.0645 | −0.1955 | −0.1575 | 0.3746 | −0.5965 | −0.1495 | −0.2535 | −0.2595 | −0.1975 | 0.0435 | −1.4045 | −1.1115 | 0.0545 | −0.2585 |
| RACGAP1 | 0.8105 | 1.2575 | 1.4215 | 0.6805 | 0.6855 | 0.6295 | 1.1595 | 0.1915 | −1.9585 | −1.8425 | 0.0755 | 0.3415 | −1.5035 | −0.5115 | −2.3925 |
| RAD17 | 2.792 | 1.359 | 1.153 | 0.684 | 2.299 | 1.305 | 2.078 | 1.298 | −0.389 | −1.178 | −1.145 | 0.152 | −0.859 | 0.227 | −0.811 |
| RAD51 | 0.236 | 0.65 | −0.261 | −0.055 | 0.182 | 0.898 | −0.333 | 1.684 | −0.28 | −0.167 | 0.012 | 0.501 | 0.779 | −0.11 | −0.103 |
| RAD51AP | 0.9285 | 1.3425 | 0.3435 | 0.8555 | −0.1155 | 0.2635 | 0.8015 | 1.3405 | −1.1165 | −1.7335 | 0.3075 | 0.3055 | −1.5155 | −0.4185 | −0.8356 |
| RAD52 | 1.054 | 0.444 | 0.545 | −0.004 | −0.538 | −0.799 | 1.154 | 0.312 | −1.469 | −1.56 | 0.034 | −0.548 | −0.776 | −0.505 | −0.946 |
| RAD54L | 0.6025 | 0.9125 | 0.5855 | 0.5205 | 0.1515 | 0.2505 | 1.1055 | 0.5375 | 0.3825 | 0.4385 | 0.7505 | 0.2665 | −0.2595 | 0.3545 | −0.2695 |
| RAMP | −1.0765 | 1.5205 | 1.9245 | 0.8755 | 2.8375 | 3.8445 | −0.5055 | 0.6145 | 0.5475 | 1.4985 | 3.5785 | 0.0736 | −0.5425 | −0.9785 | −0.4305 |
| RANBP1 | −0.4725 | −0.0615 | 1.1055 | 0.4785 | −0.1685 | −0.2525 | −2.3705 | 0.0735 | −0.3705 | 0.0385 | −0.0555 | −0.1205 | −0.4445 | −0.0455 | −0.3505 |
| RARRES1 | 1.227 | 0.024 | 0.198 | 0.425 | 0.628 | −0.548 | 0.022 | 0.624 | 0.668 | 0.398 | −0.095 | −0.852 | −0.808 | 0.414 | −0.811 |
| RBBP7 | 1.3885 | 0.6225 | 2.3875 | 0.7715 | 0.7725 | 0.5485 | 0.6355 | −0.1655 | 0.0875 | −0.2705 | 0.1215 | −0.5025 | −0.2145 | 0.0895 | −0.4765 |
| RBBP8 | −0.3276 | 0.1835 | 0.0505 | 0.9265 | 0.7265 | 0.0765 | 0.1425 | −0.8215 | −0.7095 | −0.7185 | −0.2355 | 0.6025 | −0.8955 | −0.3515 | −0.4815 |
| RBM8A | 0 | −0.92 | −0.918 | −0.444 | 0.708 | −0.579 | 1.213 | −0.611 | −0.111 | 0 | 0.572 | −0.388 | 0.27 | 0.193 | −0.452 |
| RBX1 | 1.872 | 1.104 | −0.115 | −0.01 | 0.697 | 1.275 | −0.053 | 1.085 | −0.075 | 0.274 | 0.058 | 0.595 | −0.182 | 0.886 | 0.398 |
| RDHE2 | −0.509 | −0.009 | 0.175 | −0.189 | 0.228 | −0.321 | 0.319 | −1.331 | −0.530 | −0.26 | −0.2 | 0.335 | −0.298 | −0.141 | −0.328 |
| REA | 0.285 | 0.413 | 0.394 | −0.058 | 0.094 | 0.855 | −0.148 | −0.257 | 0.115 | −0.002 | 0.002 | −0.208 | 1.015 | 0.245 | 0.046 |
| REC14 | 1.5245 | 0.4145 | 2.2885 | 0.6695 | 1.4525 | 0.6805 | 0.4585 | 2.0165 | 0.9155 | 0.8025 | 0.9215 | 0.8585 | −0.3955 | −0.0515 | −0.7655 |
| REG1A | −0.834 | −0.815 | 0.357 | −0.198 | −0.537 | −0.394 | 0.019 | −0.081 | −0.833 | −0.541 | −0.656 | −0.008 | −0.416 | −0.221 | −0.327 |
| RELB | −0.963 | −0.426 | −0.591 | −0.247 | −0.39 | −0.311 | 0.877 | −0.813 | −1 | −1.342 | −0.617 | −0.691 | −0.026 | −0.909 | −0.999 |
| REPIN1 | −1.215 | −1.455 | −1.511 | −0.791 | −1.286 | −1.56 | −1.038 | −0.325 | −0.428 | −0.243 | −0.388 | −0.9 | −0.435 | −0.551 | −0.547 |
| REPS2 | 1.5135 | 0.5935 | 1.4285 | 1.1725 | 0.5095 | 0.0855 | 0.4585 | 0.9765 | −0.8615 | −0.4525 | −0.0855 | 0.0365 | −1.3545 | −0.0755 | −0.8085 |
| RERG | 1.2235 | 0.1075 | 0.7485 | 0.4815 | 1.5475 | 0.4435 | −0.2115 | 0.2405 | −0.8525 | −0.0916 | 0.5835 | 0.0805 | −0.8715 | 0.1225 | −0.5485 |
| RFC4 | 1.3195 | 0.9225 | 0.7785 | 0.0835 | 1.9685 | 0.9085 | 0.4045 | 0.0135 | −0.8955 | −0.8155 | −0.3045 | −0.1636 | −0.3595 | 0.0565 | −0.4795 |
| RGS10 | 1.8575 | 0.3535 | 0.5105 | −0.7355 | −1.1565 | −0.5485 | −0.0385 | 0.8315 | −0.6265 | −1.2355 | 0.8916 | −0.5315 | 0.1615 | 0.0225 | −0.6785 |
| RH8DL2 | 0.447 | −0.018 | 0.139 | 0.228 | 0.286 | −0.249 | −0.581 | −0.672 | −0.172 | 0.045 | −0.099 | −0.192 | −0.325 | 0.005 | −0.134 |
| RIBC2 | 1.037 | 0.432 | 1.485 | 0.768 | 1.077 | 0.592 | −0.066 | −0.9 | −0.538 | −0.795 | −0.201 | 0.014 | −0.531 | −0.452 | −0.443 |
| RIC-8 | 0.567 | 0.214 | −0.48 | 0.858 | −0.066 | 0.622 | 0.352 | 0.194 | −0.496 | −0.701 | 0.165 | 0.371 | −0.345 | −0.393 | −0.627 |
| RIT1 | −0.273 | −0.242 | −0.142 | −0.337 | −0.414 | 0.171 | 0.114 | 0.47 | 0.524 | −0.027 | 0.215 | 0 | 0.145 | −0.114 | 0.088 |
| RLN2 | −0.561 | −1.071 | −0.245 | 0.012 | 0.411 | −0.313 | −0.383 | −0.57 | −0.963 | −0.269 | 0.012 | 0.241 | −0.833 | −0.812 | −0.114 |
| RNF10 | 0.0825 | −0.0015 | 0.0035 | −0.1455 | 0.8345 | 0.6045 | 0.0485 | −0.1635 | −0.0535 | 0.2745 | 0.1675 | −0.0385 | −0.6525 | −0.0945 | −0.8305 |
| RNF135 | 3.1995 | 1.8925 | 1.8945 | 3.4525 | 3.4635 | 3.9755 | 3.1965 | 2.6335 | 0.8395 | 2.4805 | 0.9125 | 3.6965 | 2.0145 | 3.7035 | 3.9095 |
| RNF7 | −0.049 | −0.052 | 0.403 | −0.237 | −0.271 | 0.088 | 0.669 | 0.266 | −0.902 | −1.178 | −0.306 | 0.618 | −0.355 | −0.556 | −0.485 |
| ROPN1 | −1.1085 | 1.7475 | −1.1435 | 0.1275 | 1.4295 | 1.1675 | 2.4195 | −0.1745 | 0.2835 | 0.7055 | 1.4325 | 1.8595 | 0.7055 | 0.2105 | −0.2265 |
| RPA3 | 0.7825 | −0.1775 | −0.1895 | 0.1255 | 0.5435 | −0.3895 | 0.2445 | 0.2135 | 0.1855 | 0.8135 | 0.3885 | 0.8315 | −0.5805 | 0.4875 | 0.4185 |
| RPESP | −0.62 | −0.522 | 0.481 | 0.01 | 0.285 | −0.373 | −0.226 | 1.101 | 0.834 | 0.92 | 1.235 | 1.147 | 0.818 | 1.721 | 1.215 |
| RPL11 | −0.384 | −0.514 | −0.284 | −0.476 | 0.318 | −0.411 | 0.026 | 0.755 | −0.316 | −0.09 | −0.047 | 0.75 | 0.083 | 0.855 | 0.472 |
| RPL13 | −0.275 | −0.674 | −0.163 | 0.504 | −0.302 | −1.644 | −1.133 | −0.997 | −0.137 | 0.479 | −0.353 | 0.782 | 0.345 | 0.859 | 0.948 |
| RPL13A | −0.0595 | 0.2175 | −0.2855 | 0.1785 | 0.4195 | −0.3355 | 0.6585 | 1.2085 | −0.0995 | 0.0455 | 0.5315 | 1.1385 | −0.5485 | 1.1025 | 0.8175 |
| RPL15 | −0.16 | −0.258 | 0.036 | 0.025 | −0.119 | −0.428 | −0.158 | 0.071 | −0.404 | −0.177 | 0.056 | 0.201 | 0.094 | −0.002 | 0.099 |
| RPL18A | 1.855 | 0.264 | −0.127 | 0.077 | 0.721 | −0.382 | −0.091 | −0.232 | 0.196 | 0.807 | 0.643 | 0.97 | 0.214 | 1.248 | 0.961 |
| RPL19 | 0.76 | −0.195 | 0.111 | −0.155 | 0.573 | −0.596 | −0.474 | −0.36 | 0.166 | 0.253 | 0.458 | 0.21 | −0.081 | 0.334 | −0.124 |
| RPL21 | 0.7575 | −0.2215 | −0.0245 | −0.1375 | 0.1705 | −0.6195 | −0.4185 | −0.4195 | 0.2985 | 0.3435 | 0.3045 | 0.2245 | −0.4895 | 0.1185 | −0.0985 |
| RPL23A | 0.748 | −0.242 | 0.055 | 0.03 | 1.301 | −0.297 | −0.307 | 0.059 | 0.686 | 1.102 | 0.588 | 0.923 | −0.554 | 0.389 | 0.333 |
| RPL23A | 0.3155 | −0.1165 | −0.5345 | −0.0655 | 0.4545 | −0.3775 | 0.7425 | 0.4575 | 0.0045 | −0.0265 | 0.3335 | 1.1155 | −0.0155 | 0.9895 | 0.6435 |
| RPL26L1 | 0.5555 | −0.3555 | −0.1345 | −0.0075 | 1.0945 | −0.4345 | −0.4475 | 0.0385 | −0.3455 | −0.5155 | 0.6185 | 0.7735 | 0.1285 | 0.8685 | 0.5935 |
| RPL29 | −0.0005 | −0.5455 | 0.0965 | −0.0985 | 0.5645 | −0.4455 | 0.0365 | 0.1085 | −0.2825 | 0.0485 | −0.0015 | 0.0595 | 0.1235 | 0.0595 | −0.0065 |
| RPL35 | 0.4085 | −0.0605 | 0.3665 | −0.1225 | 0.6085 | 0.2315 | −0.2255 | 0.6325 | −0.0535 | 0.0745 | 0.2545 | 0.3775 | −0.4505 | 0.8195 | 0.4095 |
| RPL36AL | −2.6105 | −0.4755 | −0.5005 | 0.6045 | −1.3835 | −1.0485 | −0.2545 | −0.3575 | 1.4065 | 0.4545 | −0.0135 | 1.1105 | 1.4605 | 1.1435 | 0.8835 |
| RPS16 | 0.2695 | 0.3895 | 1.0605 | 0.9635 | 0.1815 | 0.0515 | 0.4655 | −1.0705 | 0.4885 | 0.7385 | −0.0335 | 0.5795 | 0.0205 | 0.0375 | 0.3485 |
| RPS4Y1 | 0.1285 | −0.0285 | 1.0125 | 0.8895 | 0.6885 | 2.3405 | 1.3195 | −0.1755 | 0.6025 | −0.4365 | 1.3495 | 0.2075 | −0.4235 | 0.2495 | 0.8985 |
| RRAGA | 1.564 | 0.389 | 0.448 | 1.163 | 0.008 | 0.052 | 0.348 | 0.837 | −0.769 | −0.742 | −0.151 | −0.15 | −1.1 | −0.193 | −0.874 |
| RRAGD | 0.023 | 2.332 | 0.638 | 1.046 | −0.074 | 0.731 | 0.815 | 0.4 | −1.005 | −1.795 | 0.244 | 0.06 | −1.481 | −1.133 | −1.875 |
| RRM1 | 0.336 | −0.858 | 0.168 | −0.173 | 0.123 | −0.379 | 0.245 | −0.172 | −0.183 | −0.274 | −0.668 | −0.121 | −0.453 | 0.327 | 0.227 |
| RRM2 | 0.252 | −1.15 | 0.028 | 0.52 | −0.23 | 0.563 | −1.424 | −0.373 | 1.181 | 1.764 | 0.922 | −0.071 | 0.891 | −0.207 | 0.907 |
| RSAFD1 | 1.0545 | 1.0645 | 0.7275 | 0.3105 | 1.8535 | 0.6805 | 0.4665 | −0.5745 | 0.3555 | 0.3855 | −0.2025 | 0.4685 | −0.7515 | −0.1735 | −0.0785 |
| RSNL2 | 0.4345 | 0.7795 | 0.3835 | 0.7025 | 1.4025 | 0.7835 | 2.8045 | −0.0235 | −0.5555 | −0.4305 | −0.5585 | 0.8005 | −0.8585 | −0.0885 | −0.0855 |
| RSU1 | 0.399 | 0.44 | 0.356 | 0.993 | 0.648 | 0.574 | −0.325 | −0.602 | −0.051 | −0.605 | 0.427 | −0.856 | −0.452 | −1.196 | −0.754 |
| RTN4IP1 | 1.535 | −1.678 | 1.008 | −0.197 | −1.089 | −0.322 | −3.934 | −0.408 | −1.879 | −2.689 | −0.211 | −1.088 | −2.583 | −1.31 | −2.217 |
| S100A11 | 1.446 | −1.376 | 1.235 | 1.05 | −0.916 | −1.339 | −2.359 | −0.32 | −0.421 | −1.108 | −0.808 | 0.046 | −0.501 | −1.416 | −1.092 |
| S100A14 | −0.8545 | 0.4205 | 1.4595 | 2.8655 | 3.3345 | 2.6665 | −0.0155 | −0.1645 | 1.8985 | −0.9255 | 1.9785 | 1.0085 | −0.2635 | −1.1025 | 0.0785 |
| S100A16 | −0.4195 | 0.3545 | 0.5405 | 0.3845 | 0.3015 | −0.1115 | 0.1125 | 0.4385 | −0.0725 | −0.1585 | −0.2325 | −0.2785 | −0.4895 | −0.0865 | −0.2935 |
| S100A9 | −1.378 | −0.533 | −1.42 | −0.104 | −0.498 | −0.714 | −0.785 | −0.528 | −0.155 | 0.772 | 0.87 | 0.86 | 0.503 | 0.777 | 0.109 |
| SAE1 | 0.5505 | −0.2505 | 0.1895 | −0.2615 | 0.8145 | 0.4885 | 0.1945 | −0.0085 | −0.1445 | −1.2945 | −0.2935 | 0.2705 | 0.4025 | −0.7435 | −0.8595 |
| SALL2 | −0.072 | −0.054 | −0.047 | −0.053 | 0.264 | −0.408 | 0.051 | −0.879 | −0.491 | −1.143 | −0.819 | 0.219 | −0.106 | −0.895 | −0.58 |
| SARA1 | −0.9045 | −0.3785 | −0.3525 | −0.2985 | −0.2745 | −0.2025 | −0.5405 | −0.5235 | 0.7925 | 0.7215 | 0.3955 | 0.0585 | 0.3665 | −0.0015 | −0.1005 |
| SARA2 | −0.608 | 0.778 | −0.947 | −0.57 | 0.404 | 0.436 | −0.312 | −0.807 | 0.102 | −0.533 | 0.229 | −0.7 | −0.33 | −0.251 | −0.18 |
| SAS | −0.883 | −0.941 | −0.74 | 0.448 | −0.353 | −0.028 | 1.382 | 0.957 | 0.558 | 0.487 | 0.016 | −0.133 | 0.428 | 0.08 | −0.053 |
| SCIN | 0.2095 | 0.8965 | 0.5715 | 0.4595 | 0.9595 | 2.1365 | 1.1285 | 0.5285 | 0.6105 | 0.3005 | 0.7015 | 0.3575 | −0.2775 | 0.1355 | 0.0225 |
| SCML1 | −0.274 | −0.366 | 0.533 | −0.123 | 0.498 | 0.198 | 0.264 | −0.511 | −0.026 | −0.29 | 0.278 | −0.48 | −0.04 | −0.148 | −0.705 |
| SCPEP1 | −0.9755 | 0.8275 | −1.1305 | 0.5065 | 0.2035 | 0.3425 | −1.8176 | −0.1385 | −0.5905 | −0.9335 | −0.2245 | −0.7545 | −1.7395 | −1.7685 | −1.8915 |
| SCRIB | −0.45 | −0.066 | 0.361 | 0.615 | −1.105 | −0.697 | −0.057 | −0.069 | 1.616 | 0.797 | 1.074 | 0.925 | 0.448 | 0.347 | 0.007 |
| SDC1 | 0.383 | 0.184 | −0.003 | −0.083 | 0.41 | 1.061 | 0.095 | 0.255 | −0.786 | −0.243 | −0.398 | −1.087 | −0.625 | 0.009 | 0.191 |
| SDC3 | −0.1895 | −0.0715 | 0.3285 | 0.4975 | 0.1835 | 0.3315 | −0.1745 | 0.0425 | −0.8255 | −1.4215 | −0.5425 | 0.4255 | −0.3285 | −0.9255 | −0.8245 |
| SDCCAG0 | 0.1865 | 1.4975 | −0.0865 | 0.1225 | 1.1185 | 0.2465 | 0.2745 | 0.1835 | −0.1195 | 0.1175 | 0.3205 | −0.2875 | 0.7305 | −0.1665 | −0.2865 |
| SEC61G | 0.316 | −0.154 | 0.462 | 0.379 | 0.458 | 0.603 | 0.58 | −0.781 | −0.416 | −0.113 | −0.227 | −0.001 | −0.084 | 0.173 | −0.634 |
| SECP43 | −2.0955 | −1.4585 | −1.9405 | −0.9855 | −1.3315 | 0.5995 | −1.8955 | −0.5185 | 0.6745 | 1.0045 | 1.2455 | −0.0715 | 0.8895 | 0.5725 | 0.1325 |
| SEDLP | −1.957 | −1.915 | −0.918 | −0.173 | −1.818 | −1.005 | −0.94 | −1.777 | 0.28 | 0.548 | −1.394 | −1.166 | −1.275 | 0.264 | 0.073 |
| SEMA3B | −1.0385 | −0.8835 | −0.4135 | −0.4895 | 0.4925 | −0.2915 | −0.5335 | −0.5035 | −0.0135 | −0.3305 | 0.2385 | −0.3585 | 0.3725 | −0.5725 | 0.0425 |
| SEMA3C | 1.0555 | −0.0715 | 0.0335 | −0.1905 | 0.4315 | −0.0435 | 0.4705 | −0.3315 | −0.3805 | −0.4105 | −0.8125 | 0.0435 | −1.1865 | −0.9945 | −0.8255 |
| SEPW1 | −1.596 | −2.022 | −1.7 | −0.894 | −1.149 | −1.007 | −1.527 | −0.607 | 0.08 | −0.804 | 0.156 | 2.005 | −0.281 | 0.832 | 0.277 |
| SFRP1 | 3.1545 | 0.3675 | 1.0376 | 2.6075 | −0.6365 | 0.3755 | 0.4595 | 0.5045 | 0.2825 | 0.2975 | 0.0855 | 0.5195 | 0.5845 | 1.2615 | 0.4325 |
| SERPINA [symbol] | 1.101 | 1.92 | 1.507 | 2.798 | 1.764 | 3.141 | 2.934 | 1.947 | 1.802 | 2.639 | 1.608 | 3.757 | 1.481 | 3.447 | 4.054 |
| SERPINB [symbol] | −0.772 | 0.677 | −0.86 | −0.554 | 1.458 | 0.195 | 0.697 | 0.107 | 0.2 | −0.37 | −0.222 | 0.067 | 0.788 | 0.341 | −0.171 |
| SFRP1 | 0.236 | −0.017 | −1.404 | 0.144 | 0.495 | −0.649 | −0.564 | −1.454 | 0.038 | −0.064 | −0.602 | 0.759 | 0.804 | −0.614 | −0.044 |
| SFRS6 | −1.9355 | 2.0595 | −0.6025 | 0.4425 | 1.3365 | 1.4765 | 1.7705 | −0.8775 | 1.2305 | 2.2215 | 0.1955 | 1.1475 | −0.3415 | 0.7765 | 1.0225 |
| SGCD | 0.5125 | −0.0846 | 1.1935 | −0.1205 | 0.1185 | −0.8855 | −0.5445 | −1.1625 | −0.4425 | 0.0155 | −0.0155 | −1.3805 | −1.4215 | −0.4305 | −0.5115 |
| SGCE | 0.658 | 0.861 | 0.543 | 0.027 | 0.253 | 0.142 | 1.083 | 0.162 | −0.079 | −0.596 | −0.412 | −0.548 | 0.388 | −0.367 | −0.858 |
| SH3BGRL | −2.273 | −1.072 | −2.096 | −0.109 | −0.683 | 0.531 | −1.032 | −0.384 | −0.501 | −1.101 | 0.05 | 0.167 | 0.004 | 0.097 | −0.318 |
| SHFM1 | 0.4735 | 1.8695 | −0.1305 | 0.0025 | 0.4885 | −0.1295 | −0.4035 | 0.3855 | 0.2495 | −0.2125 | 0.7325 | 0.2405 | 1.1985 | 0.2516 | −0.1695 |
| SIAT7B | −0.271 | 1.001 | 2.048 | 1.448 | 2.941 | 0.378 | 1.014 | 1.672 | −1.246 | 0.264 | 0.669 | −0.075 | −0.032 | −0.575 | 0.029 |
| SIGLEC12 | −0.128 | −0.54 | −0.887 | −0.09 | −0.161 | −0.458 | 0.012 | 0.153 | 0.5 | 0.459 | 0.733 | 0.678 | 0.178 | 0.851 | 0.425 |
| SIT | −0.1355 | −0.6385 | 0.2825 | −0.5415 | −0.1535 | −0.6055 | −0.4425 | −1.4625 | 0.2525 | 0.1885 | 0.2115 | 0.0865 | −0.4305 | −0.2585 | −0.1395 |
| SITPEC | 0.0705 | 0.3635 | −0.3435 | −0.2045 | −0.4505 | −0.0755 | −0.1115 | 0.4245 | 0.8005 | 0.5465 | 0.7015 | −0.3495 | 0.2765 | 0.2395 | −0.0185 |
| SKP1A | 1.1805 | 0.8275 | −1.4905 | −0.2485 | 0.5975 | 0.5285 | 0.7605 | 0.8185 | −0.4015 | 0.1115 | −0.3965 | −0.0385 | −0.0085 | 0.2875 | −0.1245 |
| SLC12A9 | −2.084 | −1.117 | −1.05 | −1.153 | −0.899 | −0.725 | −1.127 | −0.598 | 0.022 | −0.307 | 0.858 | −2.049 | −0.517 | −0.342 | −1.669 |
| SLC13A3 | −0.1275 | −1.9375 | −1.7995 | −0.9345 | −1.0555 | −1.7745 | −0.9825 | −0.7465 | 1.5875 | −0.9235 | −0.4415 | −0.8385 | −1.0155 | −1.0395 | −1.5865 |
| SLC16A6 | −1.278 | 0.132 | 0.71 | −0.297 | −0.755 | −1.688 | −0.368 | −1.29 | −0.843 | −1.891 | −0.08 | 0.414 | −0.674 | −0.502 | −0.453 |
| SLC19A2 | −1.8025 | −0.7865 | −0.7515 | −0.5595 | −1.0125 | −1.8095 | 0.2675 | 0.5785 | −0.9076 | −1.0385 | −0.1625 | −0.8075 | −0.2735 | −0.6135 | −0.9215 |
| SLC1A1 | 1.2615 | 0.0335 | −0.1655 | −0.1275 | 0.6005 | 0.3695 | 0.6825 | −0.0865 | −0.4085 | −0.6185 | −0.4735 | 0.0625 | −0.6985 | −0.7415 | 0.2655 |
| SLC1A2 | −0.9345 | −1.0435 | −0.6175 | −0.8335 | −0.9875 | −0.8945 | −0.3305 | −0.2085 | −0.3425 | 0.0915 | 0.1115 | −0.2215 | −0.3185 | 0.4035 | 0.1095 |
| SLC24A3 | −0.357 | −0.182 | −0.653 | −0.303 | −0.377 | 0.113 | −0.903 | −1.247 | 0.352 | 0.199 | −0.403 | 0.354 | −0.146 | 0.001 | −0.126 |
| SLC25A29 | 0.886 | 0.758 | 0.969 | 0.954 | 0.139 | −0.35 | 0.811 | 0.284 | −0.42 | −0.69 | −0.314 | 0.682 | −0.658 | 0.094 | −0.258 |
| SLC25A4 | −1.054 | −0.961 | −0.661 | −1.181 | −0.686 | −1.112 | −1.013 | 0.598 | −0.082 | −0.469 | 0.483 | −0.382 | −1.24 | −0.606 | −1.209 |
| SLC25A5 | −0.8605 | −0.7215 | 0.5005 | 0.3325 | −0.2925 | −1.2775 | −0.6825 | 0.3375 | −0.2425 | −0.4745 | 0.1945 | 1.2025 | −0.3995 | 0.3145 | −0.1405 |
| SLC27A2 | 0.023 | −0.38 | 0.147 | −0.096 | −0.955 | −0.37 | −0.171 | 0.154 | −0.184 | −0.358 | −0.062 | 0.296 | 0.211 | 0.149 | −0.053 |
| SLC27A5 | 0.802 | −1.296 | 0.927 | −0.72 | 1.303 | 0.133 | 1.92 | 0.355 | −0.389 | −0.597 | −0.02 | −0.034 | 0.76 | −0.281 | −0.291 |
| SLC30A8 | −0.5285 | −2.1635 | −1.8725 | −1.6145 | −0.4225 | −2.2095 | −2.6475 | −2.8385 | −0.2485 | −2.1045 | −0.2025 | −2.0215 | −1.3725 | −0.0735 | −2.1235 |
| SLC39A4 | −2.588 | −1.165 | −0.587 | −0.908 | 0.125 | −1.822 | −0.81 | −0.788 | −0.805 | 0.102 | 1.473 | −0.507 | −0.687 | −0.918 | 0.053 |
| SLC39A6 | 0.7085 | −0.4005 | 0.1345 | 0.3335 | −0.0335 | −0.3575 | 0.2535 | 0.3855 | 0.8805 | 0.5505 | 0.5305 | 0.4715 | 0.5725 | 0.2945 | 0.2855 |
| SLC40A1 | 1.1785 | 0.0965 | 1.4375 | −0.4845 | 0.0145 | 0.1795 | −1.1045 | 0.7285 | −0.4355 | 0.4035 | 0.4035 | 1.0425 | −0.3165 | 1.1905 | 0.3285 |
| SLC41A3 | −0.148 | 0.04 | −0.168 | −0.067 | −0.694 | −0.677 | −0.285 | −0.873 | −0.428 | −0.343 | −0.016 | −0.265 | 0.844 | 0.34 | 0.194 |
| SLC5A6 | −1.6045 | −1.2135 | −1.3005 | −1.3655 | −1.4775 | −0.3955 | −1.2155 | −0.9245 | −1.1795 | −1.5385 | −0.5645 | −0.6905 | −1.8985 | −0.5125 | −1.0495 |
| SLC6A4 | −1.9155 | −1.2635 | −3.7405 | −1.4425 | −2.5165 | 0.5585 | 1.8075 | −1.5295 | −0.2045 | −1.0505 | 1.4225 | −2.7685 | −0.3095 | 0.2105 | 0.4255 |
| SLC9A3R1 | −0.8445 | −0.5055 | −0.7275 | −1.0535 | −0.7565 | −0.8725 | −0.9485 | 0.8455 | −1.3985 | −0.5255 | −1.1085 | −0.7965 | 0.1105 | −0.3505 | −0.7175 |
| SLITRK6 | 1.157 | −0.228 | 1.145 | 0.33 | 0.252 | 0.29 | −0.354 | −0.498 | −0.133 | −0.034 | −0.138 | −0.011 | −0.872 | −0.283 | −0.318 |
| SMA3 | −0.67 | 0.845 | 0.447 | 0.257 | −0.084 | −0.158 | 0.746 | 1.308 | −0.116 | 0.336 | 0.559 | 0.834 | 0.29 | 0.833 | 0.612 |
| SMAD2 | 0.854 | 0.874 | 1.305 | 1.121 | 0.365 | −0.072 | 0.301 | 0.051 | −0.137 | −0.295 | −0.515 | 0.4 | −1.055 | −0.282 | −0.052 |
| SMO | −0.585 | 0.114 | −0.111 | 0.004 | −0.377 | −0.577 | −0.118 | −0.285 | 0.591 | 0.318 | 0.388 | 0.223 | 1.021 | 0.189 | 0.335 |
| SMU1 | 0.834 | −0.168 | −0.039 | 0.097 | 0.7 | 0.16 | 0.325 | −0.286 | −0.382 | −0.218 | −0.4 | −0.062 | 0.282 | −0.069 | −0.202 |
| SNPH | 0.859 | 0.66 | 0.245 | 0.144 | 0.754 | −0.062 | 0.078 | 0.169 | 0.301 | 0.148 | 0.58 | −0.096 | −0.218 | 0.269 | −0.368 |
| SNRPB2 | 0.4115 | 0.1425 | 0.3885 | 0.1245 | 0.2975 | −0.1065 | 0.2995 | −0.6815 | −0.2255 | −0.3455 | 0.0035 | −0.1815 | −0.6315 | −0.1025 | −0.2875 |
| SNRPD1 | 0.737 | 1.07 | 0.418 | 0.463 | 1.491 | 1.021 | 1.389 | 0 | −0.753 | −0.965 | −0.502 | −0.185 | 0.016 | −0.704 | −0.952 |
| SNRPD3 | 1.3155 | −1.0625 | 0.3825 | 0.9595 | 0.8625 | 0.2475 | −0.4195 | 0.0605 | −0.3375 | −0.0755 | −0.2205 | 0.5175 | −0.6615 | −0.6805 | −0.2335 |
| SNRPG | 1.2516 | 1.9935 | 2.0215 | 2.1575 | 3.4195 | 2.1555 | 2.0475 | 0.6005 | 1.5985 | −0.0885 | 1.7395 | 0.8355 | 0.7665 | −0.6835 | −0.2105 |
| SNX7 | 0.6415 | −0.1445 | −0.4175 | 0.0325 | 0.1085 | −0.3385 | 0.2185 | 2.3165 | 0.1145 | −0.1435 | 0.2075 | −0.0075 | 0.3095 | 0.2565 | 0.0715 |
| SO02 | 0.0825 | 0.1285 | −0.8585 | 0.3805 | −0.0515 | −0.0565 | 0.5425 | 2.0355 | −1.2335 | −0.4805 | 0.0685 | −0.4495 | 0.4245 | 0.4845 | −0.9776 |
| SORCS1 | 0.35 | 0.655 | 0.769 | 1.406 | −1.268 | 1.035 | 1.831 | 1.058 | 0.047 | 0.526 | 0.338 | 0.508 | −0.251 | 0.782 | 0.086 |
| SOX4 | 0.1545 | −0.6915 | −0.0885 | 0.3225 | 0.4135 | 0.4465 | 0.1745 | −0.5725 | 0.1465 | 0.7945 | 0.2835 | −0.0106 | −0.3275 | 0.2785 | 0.4895 |
| SOX9 | 0.444 | 0.922 | 1 | 0.183 | −0.282 | 0.335 | 1.305 | 0.332 | −1.094 | −1.552 | 0.337 | −0.631 | −2.129 | −0.584 | −1.287 |
| SPAG18 | −0.5145 | 0.5605 | 0.5225 | −0.1885 | 0.8485 | −0.1025 | −0.2485 | −1.7945 | −0.5585 | −0.1515 | −0.5135 | 0.7185 | −1.3285 | 0.1265 | 0.3395 |
| SPAG5 | −0.1385 | −0.8415 | 0.0335 | −0.0265 | −0.6855 | 0.3955 | −0.8635 | −0.3705 | −1.0165 | −1.2045 | −0.8725 | −0.2895 | −0.3215 | 0.4045 | −0.0985 |
| SPC18 | −0.032 | −0.051 | −0.185 | 0.008 | 0.561 | 0.089 | −0.931 | −0.384 | 0.003 | 0.432 | 0.155 | 0.184 | 0.32 | 0.807 | 0.526 |
| SPFH2 | 0.276 | −0.081 | −0.396 | −0.017 | 0.33 | 0.315 | −0.088 | 0.268 | −0.337 | −0.48 | −0.485 | −0.214 | −1.159 | −0.638 | −0.484 |
| SPG3A | 1.289 | −0.385 | 0.675 | 0.375 | 0.841 | −0.195 | −0.064 | −1.135 | −1.985 | −1.985 | −1.855 | −0.207 | −3.187 | −2.495 | −2.426 |
| SPPL3 | −1.282 | −0.057 | 0.443 | 0.7 | 0.868 | −0.142 | −1.224 | −0.998 | −0.53 | −0.58 | 0 | −0.104 | −0.605 | −0.497 | −0.865 |
| SQLE | −2.396 | −1.062 | −0.175 | −0.008 | −1.159 | 0.879 | −1.825 | −2 | −1.037 | −0.783 | −0.387 | −0.484 | −0.297 | −0.45 | −0.532 |
| SQRDL | 0.7718 | 0.5485 | −0.3205 | −0.1746 | 0.0095 | −0.7399 | −1.1555 | −0.0065 | −0.1845 | 0.2885 | −0.8075 | −0.0755 | 0.7455 | −0.3555 | 0.2685 |
| SREBF1 | 2.0295 | 1.2325 | 1.3395 | 1.3645 | 0.4175 | 0.4845 | −0.1565 | 1.4505 | −0.4095 | −0.1835 | −0.2375 | −0.9715 | −0.5885 | −0.0615 | −0.9295 |
| SRI | −0.7085 | −0.1655 | −0.6875 | −0.0645 | 0.5685 | 0.3535 | −0.0815 | −0.2095 | −0.0005 | 0.4215 | −0.2875 | −1.2965 | −1.1025 | −0.4295 | −0.0345 |
| SRPK1 | 0.3255 | 1.4495 | 0.9265 | −0.0945 | 0.3445 | 0.0195 | −0.5925 | 1.2815 | −0.9405 | −0.9175 | −0.5735 | 0.4135 | −1.0115 | −0.0795 | −0.1545 |
| SSFA2 | −0.0995 | −0.7175 | 0.2215 | 0.1915 | −0.0835 | −0.4205 | −0.3585 | −0.4935 | 0.2885 | 0.2975 | −0.1715 | 0.1035 | −1.0545 | −0.5426 | −0.5415 |
| SSR4 | −0.1505 | 0.1885 | −0.4035 | −0.4875 | 0.3955 | 0.7545 | 0.1475 | 0.8135 | 0.0915 | 0.1065 | 0.1925 | 0.3795 | −0.5175 | −0.2345 | −0.0815 |
| STARD3NI | 0.1755 | 2.4265 | 2.5925 | 1.3925 | 2.2765 | 3.3805 | 2.0545 | 1.2875 | −1.6315 | −1.3265 | −1.8825 | −0.7315 | −2.8925 | −1.3775 | −1.4435 |
| STARD7 | −2.8335 | −2.0055 | −0.1835 | −1.1335 | −1.7865 | −0.5735 | −1.9045 | −0.9595 | 1.5065 | 0.3415 | 1.6095 | 0.2145 | 0.7225 | 0.8825 | −0.8075 |
| STAT1 | −0.3815 | 0.4425 | 0.1715 | 0.4845 | 0.8825 | 1.3435 | 0.2776 | 0.2405 | 0.5525 | 0.4295 | 0.4835 | 0.1555 | −0.7165 | 0.0515 | 0.0075 |
| STC2 | 0.2095 | 1.5415 | 0.8235 | 0.2855 | 0.7485 | 1.6835 | 1.7145 | 0.5265 | 0.1135 | −0.1955 | 0.4345 | −0.1685 | −0.6435 | −0.2255 | −0.5345 |
| STK24 | 1.4745 | 2.1085 | 0.2895 | 1.9105 | 0.9895 | 1.5185 | 2.6875 | 1.9275 | −1.4415 | −0.8145 | −0.2555 | 0.0165 | −1.0245 | −0.1895 | −0.9725 |
| STK6 | −0.22 | −0.413 | −0.982 | −0.389 | −0.608 | −0.418 | −0.107 | 0.214 | −0.293 | −0.657 | −0.695 | −0.223 | 0.038 | −0.524 | −0.834 |
| STMN1 | 0.1765 | −0.5215 | −0.0615 | −0.1285 | 0.3315 | 0.3185 | −0.5815 | −0.5695 | −0.2775 | −0.2975 | −0.1745 | −0.7875 | 0.0835 | −0.4905 | −1.0255 |
| STX1A | 0.4615 | 0.4445 | 0.7825 | 0.5115 | 0.7135 | 0.2625 | 1.6785 | −0.2535 | −0.8905 | −0.8395 | −0.8885 | −0.4685 | −0.8755 | −0.0565 | −0.1385 |
| STX3A | −0.7335 | 0.0745 | −0.2405 | 0.0825 | 0.3585 | 0.4765 | −0.8525 | −0.1875 | −0.3005 | −0.4795 | 0.0956 | −1.1735 | 0.7315 | 0.2645 | −0.5145 |
| STYX | −0.5895 | −1.3095 | −0.8115 | −0.3525 | 0.1205 | 0.3415 | −0.2825 | −1.1875 | −0.4035 | −0.7015 | −0.5165 | 0.4205 | −1.4635 | −0.4395 | −0.3605 |
| SUGT1 | −0.226 | 0.717 | 0.35 | 0.527 | 0.61 | 0.433 | 1.086 | 0.303 | −0.29 | 0.091 | 0.13 | −0.379 | −1.748 | 0.157 | −0.358 |
| SUMF1 | 1.8385 | 1.2425 | 0.7275 | 0.0285 | 1.1215 | 0.3355 | 1.4935 | −0.0955 | −0.8055 | −0.2775 | −0.6475 | −0.3395 | −0.1815 | 0.3795 | −0.1255 |
| SUMO2 | 0.1615 | −0.1645 | 0.8605 | −0.4415 | −0.1995 | −0.3595 | −0.1385 | −1.0095 | −1.2115 | −1.3835 | −1.0985 | −0.6635 | −1.3165 | −0.1675 | −0.2405 |
| SUV39H2 | 0.7005 | −0.3915 | −0.7535 | −0.7965 | −0.8125 | 1.4005 | 0.9885 | 0.5565 | −2.2805 | −2.0695 | −0.7985 | −3.7965 | −3.0005 | −0.5685 | −1.8055 |
| SYAP1 | −2.0235 | 0.5885 | −0.8545 | 0.4825 | −0.8145 | 0.2185 | −0.0945 | −0.6095 | 0.4465 | 0.3775 | 0.4355 | 0.9715 | 1.4165 | 0.7735 | 0.7425 |
| SYCP2 | −2.06 | −0.844 | −2.243 | −1.248 | −0.853 | −0.814 | −0.616 | −1.549 | −0.079 | 0.114 | −0.057 | −2.188 | −0.833 | −1.108 | −1.2 |
| SYNGR1 | −0.5445 | 0.1385 | −0.3605 | −0.6345 | 0.2285 | 0.3205 | −0.5815 | −0.5915 | −0.7445 | 0.1595 | −0.8965 | −1.0005 | −1.3235 | −0.1845 | −0.1155 |
| SYTL2 | 0.2585 | 0.6925 | 0.0185 | −0.2265 | −0.3665 | 0.2955 | 0.7825 | −0.4825 | 0.1435 | 0.0835 | 0.1395 | 0.0445 | 0.1245 | −0.4075 | −0.6815 |
| TAF18 | −0.7175 | −0.4955 | 0.1795 | 0.9855 | −0.2705 | 1.0335 | 0.5465 | 1.1015 | −1.0315 | −0.8165 | −0.8845 | −1.0595 | −1.6675 | −0.3125 | −0.1685 |
| TAF8 | 0.779 | 0.184 | 0.395 | 0.968 | 0.305 | −0.381 | 0.685 | 0.928 | −0.158 | −0.155 | −0.314 | −1.291 | −0.552 | 0.067 | −0.783 |
| TARBP1 | 1.415 | 0.085 | 1.24 | 0.287 | 1.195 | −0.234 | 0.216 | −0.191 | −0.876 | −0.044 | −0.582 | 0.065 | −0.165 | −0.14 | −0.345 |
| TARS | 0.0795 | −0.0905 | 0.2375 | −0.4285 | 0.0755 | −0.5135 | −0.4165 | −0.2635 | −0.8415 | −0.2855 | −0.8325 | −0.7945 | −1.4215 | −0.4205 | −0.1635 |
| TATDN1 | 0.9795 | −0.2335 | −0.4146 | −0.1925 | −0.1185 | −0.8305 | −1.0305 | −0.6285 | −0.0345 | 0.3925 | 0.2776 | −0.2975 | −0.2585 | 0.0485 | 0.0645 |
| TBC1D12 | −1.2185 | 0.5165 | −0.4035 | −0.2175 | −1.0825 | 0.2845 | −0.2215 | 0.0685 | −0.2475 | 0.0525 | 0.1815 | −0.8515 | 0.4925 | 0.4035 | 0.2875 |
| TBC1D14 | 0.022 | −0.53 | 0.218 | −0.173 | −0.28 | −0.845 | −0.466 | −0.648 | −0.483 | −0.768 | −0.355 | 0.083 | −0.368 | −0.322 | −0.386 |
| TBC1D3 | −2.1105 | −1.5415 | −2.5525 | −0.4175 | 0.2465 | −0.0435 | −0.0645 | −0.7525 | 0.0585 | 1.6605 | 1.3775 | 0.3835 | 0.3905 | −0.7965 | 0.6035 |
| TBCA | −1.84 | −1.601 | −4.414 | −0.434 | −1.593 | −2.781 | −0.79 | 0.045 | 0.3 | −0.046 | 0.421 | −0.563 | −0.34 | 0.536 | −0.88 |
| TBX1 | −0.2855 | 2.4065 | 0.3805 | −0.1455 | 0.0215 | 0.1345 | 0.2135 | 0.5975 | −0.2265 | −0.1835 | 0.0145 | 0.4205 | 0.5965 | −0.1375 | −0.3175 |
| TBX3 | −1.7 | −0.671 | −1.327 | −0.35 | −0.377 | −0.813 | −0.488 | −1.696 | −0.289 | −0.19 | 0.019 | −0.137 | 0.052 | 0.183 | −0.155 |
| TCAP | −1.004 | −0.976 | −1.6 | −0.148 | −0.762 | −0.033 | −0.614 | −2.032 | −0.207 | −0.008 | 0.235 | 0.621 | 0.683 | 0.339 | −0.447 |
| TCEAL1 | −1.1315 | −0.9505 | −2.6325 | −0.4865 | −0.3576 | −0.3975 | −0.4465 | −1.5705 | 0.4205 | 0.5615 | 0.4265 | −0.9445 | 0.4675 | −0.1935 | −0.1545 |
| TCEAL3 | −1.368 | −0.461 | 0.725 | −0.472 | −0.83 | 0.715 | 1.016 | −0.52 | −0.833 | −1.478 | −0.458 | −1.618 | −1.29 | −1.627 | −1.288 |
| TCEAL4 | 0.826 | 0.642 | 0.355 | −0.014 | 0.35 | 0.468 | 0.632 | 0.335 | −0.758 | −0.586 | 0.014 | −0.184 | −1.359 | −0.806 | −0.971 |
| TCF15 | −0.106 | −0.218 | 0.394 | 1.013 | −0.388 | 1.307 | 0.624 | 0.251 | 0.38 | 1.359 | −0.317 | 0.837 | 0.334 | 1.007 | 1.144 |
| TCF3 | 0.071 | 0.438 | 0.823 | 0.034 | 0.387 | 0.688 | 0.087 | −0.948 | 0.001 | −0.029 | −0.102 | −0.995 | −0.27 | −0.31 | −0.514 |
| TCF7L2 | −0.3825 | −0.5135 | −0.2785 | 0.0075 | −0.8855 | −0.4405 | −0.2615 | −0.1055 | 0.2375 | −0.0675 | 0.2835 | −0.0085 | −0.0285 | 0.4435 | 0.0385 |
| TDRD7 | −3.5705 | −3.8365 | −4.4785 | −1.9525 | −3.4775 | −5.1505 | −4.0615 | −2.2675 | −2.0985 | −2.1125 | 0.7006 | −1.2925 | 0.1815 | 0.2775 | −1.4625 |
| TESK2 | −0.321 | 0.494 | −0.723 | 1.937 | 1.585 | 3.31 | 1.231 | 0.823 | −0.927 | −1.164 | −0.598 | 0.095 | −1.18 | 0.071 | 2.26 |
| TFAP2B | −0.762 | 0.383 | −0.919 | 0.534 | 0.327 | −0.431 | −0.984 | −0.782 | −1.135 | −0.534 | −0.88 | 0.494 | −0.514 | 0.734 | −0.118 |
| TFCP2L1 | −3.2335 | 0.5175 | −1.2725 | −0.6865 | −1.1285 | −1.3405 | −0.9635 | −1.2255 | 0.6415 | 0.1325 | 0.6895 | −0.1965 | 0.4615 | 1.5715 | −0.0265 |
| TFCP2L2 | −4.4015 | −3.8155 | −4.3825 | −2.6855 | −4.1085 | −4.6995 | −3.2135 | −2.9125 | −0.9095 | −0.2216 | 0.2885 | 0.0325 | −0.9835 | 2.0635 | −1.9245 |
| TFF1 | 1.3965 | 0.8545 | 1.4075 | 1.6505 | 1.2005 | 0.4815 | 0.9025 | 0.9575 | −0.0045 | −1.1655 | −0.0985 | −0.3965 | −0.8885 | −0.9625 | −0.7345 |
| TFF3 | −0.731 | 0.084 | −0.792 | 0.001 | 1.254 | 0.379 | −0.262 | 0.382 | 1.499 | 2.084 | 0.277 | 0.477 | −0.136 | 1.483 | 1.343 |
| TFRC | 1.0505 | −0.2555 | −0.4585 | 0.0245 | 1.2885 | 0.7065 | 0.6435 | 0.1135 | 0.2635 | 0.6645 | 0.1715 | 0.6835 | −0.7635 | 0.1935 | 0.1385 |
| TGFBR3 | 0.85 | −1.115 | 0.075 | 0.153 | −0.436 | −0.835 | 0.338 | 0.906 | −0.794 | −0.664 | −1.135 | −1.814 | −0.939 | −0.635 | −0.786 |
| THY28 | 0.6585 | −1.6105 | −0.9435 | −0.0625 | 0.0215 | 0.1685 | −1.5405 | −0.0545 | −0.2755 | 0.9145 | 0.1805 | 1.2835 | 1.1255 | 1.0115 | 0.9895 |
| TIF1 | 0.115 | −0.893 | −0.166 | −0.53 | 0.072 | 0.338 | 0.249 | −1.158 | −1.322 | −0.94 | −1.207 | 0.407 | −1.254 | −0.297 | −0.296 |
| TIGA1 | 0.817 | 0.795 | 0.814 | 0.548 | −0.301 | 0.146 | 0.588 | 1.038 | −1.221 | −1.056 | 0.005 | 0.401 | −1.54 | −0.456 | −0.852 |
| TIM14 | −0.0025 | −0.3315 | 0.2445 | 0.4085 | 0.5475 | −0.1325 | 0.6045 | −0.6625 | −0.1855 | −0.3925 | −0.4135 | 0.5025 | −0.0035 | 0.3335 | 0.1295 |
| TIMELES [symbol] | 0.9675 | −0.8905 | 0.0485 | 0.0205 | 0.5465 | −1.3025 | −0.6425 | −0.2515 | 0.4285 | 0.1725 | −0.1715 | −0.1765 | −0.1755 | 0.9355 | 0.0555 |
| TIMM9 | −1.458 | −0.697 | 0.554 | 0.751 | 1.881 | 1.007 | 3.244 | 0.196 | 0.728 | 1.281 | −0.029 | 0.823 | 0.23 | −0.158 | 0.321 |
| TINP1 | −0.2825 | 1.0275 | 0.8935 | 0.8115 | −0.0945 | 0.3455 | 2.7115 | 0.8025 | −1.6565 | −2.1875 | 0.0225 | 0.0235 | −2.1295 | −1.3845 | −1.8555 |
| [symbol] IP1208 | −1.703 | 0.613 | −2.402 | −0.823 | 0.127 | 1.009 | −0.393 | −0.277 | 0.675 | 0.808 | 0.332 | 0.45 | 0.042 | 0.86 | 1.185 |
| TK1 | 0.554 | −0.012 | 1.088 | −0.783 | 0.925 | 1.377 | 0.393 | −0.408 | −0.316 | −0.514 | 0.053 | −0.614 | −0.897 | −0.717 | −0.079 |
| TLE2 | 2.025 | −0.301 | 1.659 | 0.27 | 0.99 | −0.698 | −1.231 | 0.476 | −0.155 | 0.646 | −0.895 | −0.035 | −0.849 | −0.526 | 0.417 |
| TLR5 | −1.841 | −1.084 | −0.873 | −1.017 | −0.922 | −1.841 | −0.139 | −1.838 | −1.263 | −1.702 | −1.024 | −0.899 | −0.8 | −0.602 | −1.095 |
| TM4SF12 | 0.239 | 0.852 | 1.325 | 1.61 | 0.91 | 0.821 | 0.373 | −0.116 | −0.668 | −0.92 | −1.091 | 0.102 | −2.208 | −1.026 | −0.878 |
| TM4SF13 | −0.772 | −2.877 | −3.25 | −1.192 | −1.321 | 0.707 | −0.337 | −2.252 | −0.304 | −0.919 | −0.905 | −0.178 | −1.282 | −0.803 | −1.431 |
| TMEM14A | 0.5985 | 0.7625 | −0.3475 | −0.1335 | −0.0195 | 0.3885 | −0.4375 | 0.1515 | 0.0815 | −0.0485 | 0.3595 | 0.3175 | −0.6735 | 0.0435 | −0.2605 |
| TMEM25 | 1.0725 | −0.1085 | −0.0185 | 0.5845 | −0.5855 | 1.0485 | −0.5945 | 1.4695 | −0.2345 | 0.3055 | 0.1925 | 0.0995 | 0.6035 | 0.5465 | 1.0295 |
| TMEM4 | 0.302 | −0.068 | 0.683 | 0.183 | 1.288 | 1.848 | −0.364 | 0.939 | −0.345 | −0.2 | 0.417 | −0.401 | −1.314 | −0.161 | −0.732 |
| TMPRSS5 | 1.81 | 0.81 | 0.181 | 1.715 | 1.64 | 1.487 | 4.26 | 0.945 | −0.288 | 0.015 | 0.068 | 1.32 | 0.78 | 0.75 | 0.24 |
| TMSB10 | 0.123 | 3.241 | 3.737 | 1.235 | 1.726 | 0.857 | 0.649 | 2.95 | 0.219 | 0.294 | 0.852 | 1.831 | 0.277 | −0.209 | 0.282 |
| TMSNB | 0.4835 | 0.7595 | 2.9165 | 0.3625 | 1.1145 | 2.0275 | 0.4075 | 0.0355 | −0.0145 | −0.0925 | −0.2285 | 1.3005 | 0.3965 | 0.7115 | 0.6455 |
| TNFRSF1 [symbol] | 0.815 | 0.299 | 2.884 | 0.494 | 1.089 | 2.235 | 0.243 | 0.634 | −0.008 | −0.352 | −0.117 | 1.073 | 0.271 | 0.713 | 0.496 |
| TNFRSF2 [symbol] | 0.2345 | 0.9405 | 0.7445 | −0.1565 | 1.5035 | 0.4315 | −0.0865 | 1.0776 | −1.4425 | −1.3085 | −1.1395 | −0.2275 | −1.1845 | −1.0195 | −0.8605 |
| TNFRSF2 [symbol] | −0.8585 | −0.5685 | −0.4255 | −0.7135 | −0.2865 | −1.6215 | 0.0175 | −0.0005 | 0.6955 | 0.4305 | 0.1025 | 1.0995 | 0.6305 | 3.0475 | −0.2345 |
| TNFSF4 | −1.1816 | −0.2525 | −0.8245 | −0.6885 | −0.9825 | −0.9935 | −0.7205 | −1.1015 | −0.3075 | −0.1445 | −0.3985 | −1.3125 | −0.6085 | −0.1255 | −0.0645 |
| TNFSF7 | −0.4485 | −0.1395 | −0.2775 | −0.3135 | 1.5435 | 1.3375 | 0.0785 | 0.1845 | 0.0025 | 1.1885 | 0.7185 | 1.6485 | 0.8755 | 0.6295 | 0.7295 |
| TNIK | 0.7415 | 0.1055 | 0.4445 | 2.4405 | 0.4345 | 4.7755 | 0.9615 | 0.6015 | 0.5735 | 0.2025 | 0.5715 | 1.4895 | 0.5015 | 2.3875 | 2.1435 |
| TNNC2 | 0.084 | 0.029 | −0.854 | −0.063 | 0.838 | −1.157 | 1.291 | 1.813 | −0.559 | −1.044 | 0.787 | 0.354 | 1.286 | −0.222 | 0.11 |
| TNNI2 | −1.224 | −0.499 | −0.884 | −0.495 | −0.781 | −0.296 | −0.512 | −1.358 | 0.17 | −0.437 | 0.293 | −0.707 | 0.312 | 1.148 | −0.09 |
| TNNI3 | 0.3125 | −0.9645 | 0.8955 | −0.1125 | −2.0735 | 0.2235 | −0.7655 | −0.6315 | −1.3315 | −1.5275 | −0.7685 | −0.7165 | −1.1425 | 0.0145 | −0.8865 |
| TOB1 | −0.4576 | −1.1125 | −0.3985 | −0.5175 | 0.0955 | −0.8105 | −0.5815 | 0.6525 | −0.2225 | −0.0176 | 0.0505 | 0.7995 | 0.6305 | 0.3905 | 0.5485 |
| TOM1L1 | 1.4735 | −0.5355 | 0.4425 | 0.2655 | 0.8045 | −0.0825 | 0.0855 | 0.0485 | 0.1775 | −0.1555 | 0.9235 | 0.1915 | 0.3145 | 0.9135 | 0.0275 |
| TOMM7 | 0.7675 | 3.1085 | 1.6275 | 1.0405 | 0.5415 | 0.9495 | 2.0225 | 0.5285 | −0.3045 | −0.3535 | −0.1385 | −0.8405 | −0.8185 | −0.5915 | −1.1835 |
| TOP1MT | 0.355 | −0.348 | 0.351 | 0.581 | −0.078 | −0.504 | 0.272 | −0.384 | −0.215 | 0.049 | −0.034 | −1.475 | −0.532 | 0.347 | −0.006 |
| TOP2A | 0.7315 | 0.3085 | 1.0435 | 0.0505 | 0.2035 | 0.8085 | 1.3655 | 0.9865 | −0.9085 | −0.4325 | −0.3705 | −1.1715 | −1.3125 | −0.4005 | −0.7825 |
| TOP2B | 2.076 | 0.09 | 1.574 | 1.49 | 0.147 | 1.827 | 2.745 | 0.322 | −2.046 | −1.785 | −0.961 | 0.184 | −1.587 | −0.72 | −2.382 |
| TOPBP1 | −0.6115 | 0.1285 | −1.4025 | −0.9935 | −0.4445 | −0.6885 | −1.8485 | −0.8435 | −0.7835 | −1.3285 | 0.2075 | −0.6035 | 0.1085 | −0.3605 | −1.0595 |
| TOPK | 1.9805 | 1.4055 | 0.7875 | 1.3085 | 1.5055 | 0.5105 | 0.8285 | 0.3955 | −0.8175 | −0.0415 | −0.9045 | −0.5155 | −1.0225 | −0.9755 | −0.7745 |
| TP53AP1 | −1.6275 | −0.7805 | −0.5816 | −0.8285 | −0.1145 | 0.3115 | −1.3595 | −0.0115 | −0.4795 | −0.8305 | −0.5785 | −1.7585 | −1.4165 | −0.6295 | −0.9445 |
| TP53BP2 | 0.3175 | −0.2695 | 0.3535 | 1.1455 | −0.2595 | −0.3215 | 0.0085 | −0.0485 | −0.4805 | −1.0435 | −0.5355 | 1.2145 | 0.0905 | 0.3165 | 0.9155 |
| TP53INP1 | 1.3635 | 1.1515 | 1.1765 | 0.3645 | 0.9185 | 0.5725 | 0.8115 | −0.0595 | −0.6945 | −0.4865 | −0.1755 | 0.8835 | −0.5045 | 0.2105 | −0.2705 |
| TPD52L1 | −0.341 | 0.817 | 1.814 | 0.545 | 2.518 | 0.794 | 0.977 | 0.821 | −1.533 | 1.072 | 0.538 | −0.644 | −1.049 | −0.587 | −0.154 |
| TPRT | 0.0185 | −0.4385 | −0.7695 | −0.2815 | 0.0115 | −0.3825 | −0.3565 | 0.8065 | −0.2645 | −0.7975 | −0.0405 | 0.2535 | −0.4055 | 0.4015 | −0.5035 |
| TRA@ | −0.212 | −0.138 | −0.507 | −0.237 | 0.748 | 0.934 | 0.15 | −0.56 | −0.136 | −0.187 | −0.252 | 0.19 | 0.133 | 0.097 | −0.14 |
| TRAF4 | −0.792 | −0.257 | −0.599 | −0.367 | −0.173 | −0.383 | −0.287 | 0.034 | 0.23 | 0.121 | 0.058 | 0.133 | 0.797 | −0.083 | −0.381 |
| TRAPPC4 | 0.441 | 0.678 | 0.596 | 0.187 | 0.973 | 1.787 | −0.685 | −0.118 | −0.345 | 0.965 | 1.106 | −0.128 | 0.656 | −0.398 | −0.585 |
| TRH | 1.8125 | 0.0595 | 0.1165 | 1.3255 | 0.6795 | 2.6305 | 0.8065 | 0.1845 | 0.6305 | 1.2835 | 0.0985 | −0.5095 | 0.8695 | 1.9345 | 1.4115 |
| TRIM17 | −0.4415 | −0.1775 | 0.1875 | 0.1235 | 0.3855 | 0.5655 | −0.4125 | −0.5565 | −0.0775 | 0.7025 | −0.1015 | −0.1865 | −0.2248 | −0.1615 | −0.0475 |
| TRIM2 | 0.5825 | −0.5325 | −0.1865 | −0.1545 | 0.6245 | −0.1385 | −0.7495 | −0.2505 | −0.6346 | −0.1675 | 0.0005 | 0.3735 | −0.1605 | −0.2585 | −0.6885 |
| TRIM6 | 0.474 | −0.772 | −0.419 | 0.209 | 0.003 | 0.748 | −0.119 | 0.062 | −1.646 | −1.094 | 0.027 | −2.335 | −1.302 | −0.804 | −1.4 |
| TRIP3 | 0.0295 | −0.5935 | −0.2725 | 0.0485 | 0.2725 | −0.2105 | 0.4975 | −0.4715 | −0.0695 | −0.0375 | −0.1885 | −0.1395 | −0.5165 | −0.5125 | −0.4785 |
| TRPS1 | 0.4515 | −0.7355 | −1.0285 | −0.3255 | −0.2325 | 0.0625 | −0.9025 | −0.4495 | −0.8205 | −0.1175 | −0.8975 | −0.6085 | −0.5895 | −0.2565 | −0.3365 |
| TSG101 | 0.4135 | −0.8455 | −1.2355 | −0.2925 | −1.2755 | −0.1055 | −0.9855 | −0.3315 | −0.7125 | −0.7375 | −0.7165 | 0.1845 | −0.6345 | −0.2815 | −0.7885 |
| TTC10 | 1.5145 | 1.1955 | 1.4995 | 1.6165 | 1.4825 | 1.1385 | 2.4175 | 1.2965 | −0.7515 | −1.3525 | −0.5026 | −0.9865 | −1.5285 | −0.2395 | −1.3465 |
| TTC12 | 1.975 | 1.111 | 1.09 | 0.497 | 0.655 | 1.427 | 0.01 | 1.712 | −0.048 | −0.431 | 0.635 | 0.508 | 0.453 | 0.686 | 0.309 |
| TTK | −0.8455 | 2.6265 | 0.2895 | 1.7655 | −0.3025 | 3.1455 | −0.9535 | 2.1815 | 0.8515 | 1.6435 | 0.6955 | 2.4345 | 1.7715 | 2.6785 | 2.8085 |
| TTLL4 | 0.295 | 1.219 | −0.012 | 0.836 | −0.143 | 0.477 | −0.342 | 0.687 | 1.1 | 0.583 | 1.118 | −0.871 | 1.658 | −0.227 | −0.707 |
| TTYH1 | 0.3775 | −0.2825 | −1.0605 | −0.8965 | −0.3155 | 1.2095 | −0.3005 | 1.3905 | 0.3085 | 0.7115 | 0.7585 | −0.7885 | −0.1856 | 0.1235 | 0.3035 |
| TUBB3 | 1.2345 | −0.0945 | 0.2405 | 0.0795 | 0.3755 | 0.8875 | 0.6846 | −0.4585 | −0.2835 | −0.5035 | 0.1745 | 0.0125 | −0.9795 | −0.4585 | −0.8975 |
| TULP3 | 1.746 | 2.325 | 1.005 | 1.225 | 0.18 | 1.385 | 2.387 | 0.525 | −0.995 | −1.075 | 0.415 | 0.745 | −1.838 | −0.145 | −1.248 |
| TXN | 0.588 | 0.178 | 0.075 | 0.547 | 0.213 | 0.4 | −0.288 | 0.021 | 0.014 | −0.146 | 0.873 | 0.178 | −0.695 | −0.348 | −0.397 |
| TYMS | 0.7345 | 0.6925 | 0.7445 | 0.7755 | 1.1245 | 0.9515 | 0.6075 | 0.9025 | −0.0745 | 0.4075 | 0.0595 | 0.9655 | 0.5515 | 0.4185 | 0.4005 |
| U5-116KD | 0.3635 | 0.1655 | 0.5505 | 0.0295 | 0.2925 | −0.0555 | 0.2005 | −1.0715 | −0.4785 | −0.7725 | −0.1395 | −0.1895 | −0.6995 | −0.5925 | −0.8295 |
| UBE2E3 | −1.077 | 0.13 | −0.393 | −0.645 | −0.107 | 0.531 | −0.12 | 0.362 | −0.522 | −0.316 | −0.52 | −0.427 | −0.918 | 0.001 | 0.006 |
| UBL5 | −1.1415 | 4.2085 | 0.8085 | 0.0255 | 0.2935 | 0.7785 | 0.1025 | 2.6835 | 0.8535 | −0.4605 | −0.5305 | 0.0035 | 0.0985 | −1.0485 | 1.3255 |
| UBN1 | 0.945 | 0.043 | −0.144 | 1.024 | 0.464 | 0.942 | −0.428 | −0.552 | −0.607 | −0.581 | −0.68 | 0.123 | −1.319 | −0.386 | −0.388 |
| UCHL1 | −0.513 | −0.732 | −0.43 | −0.544 | −0.114 | −1.122 | −0.337 | 0.088 | 0.534 | −0.481 | 0.887 | −2.361 | 0.138 | −1.222 | −0.869 |
| UCHL3 | 0.339 | 1.443 | 0.752 | −0.486 | 0.794 | 1.063 | 0.868 | 0.271 | −0.871 | −1.836 | −0.998 | −1.181 | −0.913 | −0.8 | −1.162 |
| UGCG | −0.9785 | −0.5075 | −1.2575 | 1.7765 | −0.4715 | −0.0855 | −0.4905 | −0.8705 | 0.2716 | −0.3095 | 0.0085 | 1.8285 | 2.1355 | 2.9415 | 0.6005 |
| UGCGL1 | −0.7545 | 0.5835 | 0.2885 | 0.3215 | 1.3435 | 1.8825 | 1.0265 | 0.8155 | −0.2825 | 0.2315 | 0.5485 | −0.2335 | 0.1645 | −0.3395 | −0.1055 |
| UGT2B7 | −0.2185 | −0.0265 | 0.6315 | −0.4345 | 1.1205 | 1.1295 | −0.9845 | −0.8035 | −1.2415 | −1.2855 | 0.1255 | −0.5295 | −0.5695 | −0.1385 | −0.4695 |
| UNC13D | −0.187 | −0.258 | −0.249 | −0.224 | −0.464 | −0.686 | 0.162 | −1.304 | 0.428 | 0.698 | −0.317 | 0.015 | 0.137 | 0.208 | 0.335 |
| VAMP8 | −0.708 | 0.429 | 1.423 | 0.43 | 1.58 | −0.213 | 0.093 | 0.246 | −0.094 | 0.045 | 0.58 | −0.485 | −0.287 | −0.42 | 0.168 |
| VAPA | −2.1925 | −0.1075 | 0.4415 | −0.3645 | −2.1805 | −1.5735 | −2.2285 | −2.3475 | −1.2275 | −1.6005 | 0.2895 | −1.8625 | −0.5485 | −0.8645 | −1.6035 |
| VAV1 | −0.84 | 0.023 | −0.666 | −0.202 | −1.091 | −0.52 | −1.164 | 2.016 | −0.126 | −0.33 | −0.322 | 0.51 | 0.818 | 1.885 | 0.306 |
| VAV3 | 0.484 | −0.078 | 0.211 | 0.227 | −0.15 | 0.357 | −0.205 | −0.474 | 0.239 | 0.07 | −0.139 | 0.371 | −0.244 | −0.002 | 0.029 |
| VCX3 | 0.029 | 0.059 | −0.588 | −0.083 | 0.152 | −0.228 | 0.044 | −0.325 | 0.133 | 0.181 | −0.047 | 0.384 | 0.753 | −0.126 | 0.18 |
| VDAC2 | 0.8205 | 0.7025 | 1.3185 | 0.8785 | 2.1725 | 2.7885 | 1.8145 | −0.5715 | −0.0215 | 0.3885 | 0.4885 | 0.2055 | 1.0055 | 0.9885 | 0.2595 |
| VEST1 | 0.397 | −0.034 | −0.069 | 0.126 | 0.419 | −0.409 | −0.308 | −0.147 | 0.143 | −0.039 | 0.586 | 0.638 | 1.401 | 0.555 | 0.482 |
| VGLL1 | −0.8485 | −0.2935 | −0.7635 | 0.1875 | 0.0735 | 0.0465 | −0.7205 | −0.7405 | 0.8645 | 1.4135 | 0.9095 | 1.3985 | 2.1045 | 1.1515 | 1.1215 |
| VMD2L2 | −1.314 | 0.459 | −1.009 | −0.286 | 0.578 | 1.316 | 0.363 | −1.477 | 0.796 | 0.411 | 0.947 | −0.433 | 0.312 | 0.027 | −0.172 |
| VSX1 | 0.352 | −0.062 | −0.04 | −0.089 | 0.072 | 0.608 | 0.448 | −0.858 | −0.559 | −0.668 | −1.021 | −0.452 | −1.143 | −0.9 | −0.668 |
| WARP | −0.684 | −0.076 | 0.004 | 0.176 | −0.388 | −0.212 | −1.241 | 0.004 | −0.075 | −0.008 | 0.124 | −0.289 | 0.588 | 0.951 | 0.537 |
| WDR26 | 0.1355 | −0.4355 | 0.0435 | 1.0155 | −0.7375 | 0.5055 | 0.0275 | 0.2585 | 0.0965 | 0.4355 | 0.7935 | −1.1925 | −1.6875 | −0.2295 | −0.2195 |
| WDR6 | −0.58 | −0.019 | −0.355 | −0.538 | −0.077 | −0.476 | 0.494 | 0.083 | −0.358 | −0.449 | 0.142 | −0.371 | −0.344 | 0.192 | −0.572 |
| WHSC1L1 | 3.038 | 2.308 | 1.891 | 1.141 | 1.89 | 2.521 | 1.005 | 1.961 | 0.931 | 0.891 | 0.251 | 0.572 | 1.02 | 0.571 | −0.015 |
| WNT3 | 1.0015 | −0.1565 | 0.1895 | 0.1255 | 0.2245 | 0.8935 | 0.7055 | 0.0125 | 0.4265 | −0.0095 | 0.3445 | −0.5065 | −1.6245 | −1.0895 | −0.8765 |
| WNT6 | −1.0965 | −1.1235 | −1.2465 | −1.2075 | −0.2805 | −0.2225 | −1.0515 | −0.9605 | −0.4775 | −0.5855 | −0.0875 | −2.7115 | −2.0125 | −0.4795 | −0.5805 |
| WSB2 | −0.5225 | 0.4065 | −0.2745 | −0.0016 | −0.3315 | 0.2245 | 1.1005 | 0.1775 | −0.0525 | 0.7565 | 0.0885 | 0.2985 | 1.1745 | −0.0965 | 0.5115 |
| WWP1 | −3.2975 | −0.5675 | −0.9185 | −2.0025 | −2.4325 | −2.5555 | −3.0175 | −0.8265 | −0.3625 | −2.1875 | −0.1865 | −1.3305 | −1.5175 | −0.1045 | −2.3995 |
| WWP2 | −0.038 | 0.24 | 1.155 | −0.22 | −0.756 | −0.459 | −0.792 | −1.053 | −0.345 | −0.404 | 0.063 | 0.031 | 0.921 | 0.461 | −0.178 |
| XBP1 | −1.022 | 1.134 | −1.148 | 0.026 | 0.207 | 0.176 | 0.526 | 0.343 | 0.012 | −0.242 | 0.011 | −0.264 | 0.923 | 0.262 | −0.035 |
| XYLT2 | 0.0275 | 0.4045 | 0.4585 | −0.0735 | 0.2925 | 0.2686 | −0.2725 | −0.8475 | −0.6335 | −0.7925 | −0.8325 | 0.1235 | −0.8125 | −0.5645 | −0.4185 |
| YBX2 | −0.144 | 0.385 | 0.831 | −0.131 | 0.71 | −0.646 | 0.038 | −0.6 | −0.874 | −0.381 | −0.925 | 0.007 | −1.864 | −0.269 | −0.37 |
| YIF1 | −1.4985 | −0.9245 | −0.1155 | 0.0465 | 0.1085 | −0.6595 | −0.0495 | −1.4895 | 1.0085 | −0.1745 | −1.0915 | 0.6785 | −0.8955 | −0.9475 | 0.4655 |
| ZA20D3 | 0.7375 | −1.3195 | 0.3175 | 0.3125 | −0.2835 | 0.0295 | −0.6395 | 0.2735 | 0.1145 | 0.0285 | −0.2455 | 0.4905 | 0.2065 | 0.4005 | 0.4375 |
| ZAP128 | −1.2825 | −0.9055 | −0.7885 | −0.4215 | 0.1265 | −0.1065 | −0.8585 | −0.4225 | 0.5545 | 0.8885 | 0.0905 | 0.0595 | 0.7915 | −0.1265 | 0.3645 |
| ZBED3 | 0.666 | −0.172 | 0.092 | −0.142 | 0.332 | 0.297 | −0.328 | 0.384 | 0.477 | 0.056 | 0.312 | −0.481 | −0.325 | −0.253 | −0.739 |
| ZBTB4 | 0.264 | −0.211 | 0.182 | 0.232 | 0.002 | 0.465 | 0.289 | −0.016 | 0.208 | −0.002 | −0.328 | −0.173 | −0.513 | −0.657 | −0.5 |
| ZCWCC1 | 1.4985 | 0.7745 | 0.5785 | 0.9575 | 0.6815 | 0.7315 | 1.4725 | 0.7185 | −1.3005 | −0.7925 | −0.7455 | −0.3085 | −0.9485 | −0.5535 | −0.4565 |
| ZCWCC2 | −0.065 | −0.747 | −0.446 | −0.2 | −0.289 | −0.743 | −1.139 | 0.883 | 0.342 | 0.725 | −0.071 | 0.352 | −0.108 | 0.497 | 0.366 |
| ZDHHC13 | −1.1765 | −0.8165 | 0.2015 | −0.8195 | 0.0475 | −1.2505 | −1.8225 | −0.1995 | 0.0615 | 0.9445 | −0.5495 | −0.0465 | 0.0645 | −1.1915 | 0.2425 |
| ZDHHC14 | −0.4725 | −0.7305 | −0.5075 | −0.5175 | −0.1065 | −0.2585 | −0.2635 | −1.8085 | 0.5445 | 0.6095 | −0.0655 | 0.8095 | 0.8135 | −0.0405 | 0.1115 |
| ZFHX4 | 0.4835 | −0.5485 | −0.2885 | 0.3705 | −0.5045 | 0.2415 | −0.0985 | 0.3115 | −0.8015 | 0.2275 | 0.2965 | 0.5735 | 0.7825 | 0.4175 | 0.1455 |
| ZFYVE21 | 1.537 | −0.208 | −0.561 | 0.716 | 1.324 | 0.793 | 0.383 | 0.052 | −0.433 | −1.043 | −0.762 | −1.498 | −0.584 | −0.187 | −0.919 |
| ZNF133 | −0.523 | −0.412 | −0.693 | −0.522 | −0.438 | −0.8 | −1.143 | −0.346 | −0.160 | 0.247 | −0.358 | −1.97 | −0.752 | 0.46 | −0.131 |
| ZNF165 | 0.8185 | 1.0815 | 0.3985 | 0.1405 | 0.6755 | 0.4885 | −0.1055 | 0.2855 | −0.4215 | −0.3185 | −0.2885 | −0.4045 | −0.4745 | 0.2665 | −0.1655 |
| ZNF24 | 0.387 | −0.007 | 0.538 | 0.107 | 0.758 | 0.523 | −0.203 | −0.638 | −0.696 | −0.138 | −0.961 | −0.101 | −1.479 | −0.48 | −0.4 |
| ZNF318 | −0.0845 | 0.2085 | 0.2175 | −0.0465 | 0.0835 | 0.2175 | 0.0875 | −0.0545 | 0.9995 | 0.1255 | 0.1395 | −0.4205 | 0.2415 | 0.1135 | −0.2855 |
| ZNF54 | 0.7185 | 1.2685 | 0.6505 | 0.7325 | 0.3875 | 1.7045 | 1.7735 | 1.9055 | 0.4545 | 1.1015 | 0.0175 | 0.0645 | 0.6936 | 1.3375 | 1.3225 |
| ZNF432 | 0.3495 | 0.0795 | 0.4615 | 0.4475 | 0.1485 | −0.5155 | 0.4305 | 0.1575 | −0.7285 | −0.5785 | −0.7865 | −0.7305 | 0.3315 | 0.0885 | −1.0845 |
| ZNF482 | −1.029 | −1.268 | 0.259 | −0.014 | −1.994 | 0.04 | −0.575 | −1.377 | −1.938 | −1.916 | −0.421 | −0.412 | −0.374 | 0.451 | −0.212 |
| ZNF505 | −0.518 | −1.015 | −0.825 | −0.127 | −0.376 | −1.212 | −0.726 | −0.301 | 0.461 | 0.638 | 0.865 | 0.289 | 0.399 | −0.043 | 0.42 |
| ZNF552 | −1.1005 | −1.2115 | −0.6425 | −0.4725 | −0.9726 | 0.1665 | −0.4785 | −0.4355 | −1.6515 | −1.2595 | −0.0755 | −0.9485 | 0.0665 | 0.0585 | −0.8415 |
| ZNF577 | −0.1205 | 0.2545 | −0.1815 | −0.1495 | −0.6025 | −0.8795 | −0.6475 | −0.4295 | −0.1226 | 0.1995 | 0.1145 | −0.4485 | −0.1845 | −0.2145 | −0.6585 |
| ZNF587 | −0.6045 | −0.5365 | −0.0935 | −0.2825 | −0.0335 | 0.3845 | 1.2545 | 0.5825 | −0.7795 | −0.3185 | −0.3915 | 0.4116 | −0.1485 | −0.0975 | −0.2715 |
| ZNF598 | 0.0595 | −0.2095 | −0.0005 | 0.2215 | −0.3235 | 0.6795 | −0.1825 | −0.4885 | −0.9895 | −0.3925 | −0.5415 | −0.7055 | 0.2495 | −0.0655 | 0.3785 |
| ZNF599 | 0.521 | −0.058 | 0.294 | 0.12 | 0.815 | 0.28 | 0.306 | 0.108 | −0.143 | 0.037 | −0.487 | −0.522 | 0.208 | 0.003 | −0.01 |
| ZNF606 | 0.995 | 1.839 | 0.487 | 0.487 | 0.85 | 0.868 | 0.808 | −0.22 | −0.318 | −0.029 | −0.513 | −0.145 | −0.078 | −0.325 | −0.372 |
| ZNF614 | 0.834 | −0.251 | 1.095 | −0.122 | 1.228 | 0.437 | 0.618 | −0.109 | −1.34 | −0.067 | −0.128 | 0.033 | −1.029 | −0.428 | −0.383 |
| ZNF639 | | | | | | | | | | | | | | | |
| ZNF7 | | | | | | | | | | | | | | | |

| | Sample | | | | | | | | | |
|---|---|---|---|---|---|---|---|---|---|---|
| | Normal Br | Normal Br | BR03-724 [symbol] | 9830-0004 | 9830-0305 | BC00014 | 9830-0004 | 9830-0304 | PB339 | PB271 |
| | Class | | | | | | | | | |
| Symbol | NORMAL-I | NORMAL-I | LUMINAL | LUMINAL | LUMINAL | LUMINAL | LUMINAL | LUMINAL | LUMINAL | LUMINAL |
| | −0.3185 | −1.1445 | −0.4775 | 0.4895 | 1.5095 | 0.1995 | 0.6175 | 1.5795 | 0.5815 | 0.1165 |
| | −0.219 | 0.096 | −0.030 | −0.105 | 0.368 | −0.267 | 0.214 | −0.012 | 0.041 | −0.109 |
| ABAT | −0.483 | −2.012 | −1.958 | 2.276 | 0.346 | 2.285 | 1.529 | 1.578 | −0.875 | 0.909 |
| ABC1 | −0.0925 | 0.0835 | −0.4765 | −0.5155 | 0.0965 | −0.8325 | 0.3865 | 0.2955 | 0.0585 | −0.1885 |
| ABCC11 | −0.4055 | −0.2135 | 0.5955 | 0.9985 | 0.3215 | −0.2355 | 0.0075 | −0.5055 | −0.9045 | −0.4475 |
| ABCC3 | 0.187 | 0.308 | 0.721 | −0.111 | 0.76 | 0.874 | 0.489 | 0.148 | −0.026 | 0.014 |
| ACAS2L | −0.2855 | −0.1435 | −0.6825 | −0.5435 | −0.7135 | 0.2145 | −0.5085 | 0.1615 | −0.1145 | −0.3055 |
| ACAT1 | 0.011 | −0.368 | 0.758 | 0.642 | 1.463 | 0.289 | 1.772 | 1.121 | 1.212 | −0.210 |
| ACAT2 | −0.0675 | −0.3635 | −0.1525 | −0.2935 | 0.5595 | 0.9325 | 0.6235 | 0.8505 | 1.2615 | 0.4555 |
| ACOX2 | 0.4405 | −0.2015 | −0.0585 | 0.3785 | −0.6275 | 1.4265 | 0.6175 | 0.2615 | −0.2975 | −0.3565 |
| ACDX3 | 0.1095 | −0.2935 | −0.9615 | −1.0365 | 0.1725 | −0.1095 | −0.7755 | −0.7905 | 3.5055 | 0.1395 |
| ACSL3 | −0.521 | 0.13 | −0.086 | −0.214 | 0.479 | 0.011 | −0.616 | −0.743 | 0.251 | 0.05 |
| ACTA1 | −0.0215 | 0.1505 | 0.0685 | −0.1315 | 0.0845 | −0.2495 | −0.2425 | −0.2695 | 0.0035 | 0.1245 |
| ACTB | −1.3495 | −1.4945 | −0.4525 | 1.2425 | 0.2585 | −0.3265 | 0.0405 | −0.8925 | 0.5365 | −0.1075 |
| ADAM33 | 0.198 | 0.311 | −1.246 | −0.846 | −1.05 | 2.089 | −0.573 | −0.08 | −1.333 | −0.478 |
| ADAMDE [symbol] | −0.3135 | −0.1976 | −0.5425 | −0.1735 | 0.1365 | 0.1815 | 0.0115 | 0.7265 | 0.8265 | −0.2835 |
| ADPN | −0.0395 | 1.2025 | −0.0095 | 2.1175 | 0.9435 | 1.5795 | −0.1315 | −0.5235 | −0.6025 | 0.2325 |
| ADPRHL1 | −1.4875 | −1.5055 | 4.3775 | 0.6715 | 2.5565 | −2.0955 | 1.2835 | 1.8375 | −1.2305 | −0.0585 |
| ADRA2C | −0.3755 | 0.6175 | 0.1475 | −0.1445 | −0.0845 | 0.1935 | −0.3245 | −0.7975 | 1.0295 | −0.0865 |
| AGR2 | −0.1795 | −0.0595 | 0.3055 | 0.0715 | 0.7415 | 0.7005 | 0.5015 | 0.8185 | 0.0405 | 0.0205 |
| AGT | −0.403 | −0.024 | 0.616 | −0.045 | −0.152 | −0.021 | −0.19 | 0.01 | 0.054 | −0.151 |
| AGTR1 | −0.145 | 1.053 | 0.413 | −0.57 | 0.757 | 0.528 | 0 | 0.612 | 0.403 | 0.484 |
| AHCYL1 | −0.077 | 0.282 | 1.066 | 0.329 | −0.237 | 1.046 | 0.37 | 0.302 | 0.977 | 1.058 |
| AHNAK | −0.064 | 0.166 | 0.94 | 0.304 | 0.45 | −0.232 | 0.769 | 0.535 | −0.282 | −0.359 |
| AHR | −0.0995 | −0.3475 | −0.1316 | 0.5116 | −0.0235 | 0.1065 | 0.0915 | −0.0355 | 0.2195 | −0.0375 |
| AIM1 | 0.0165 | 0.8515 | 0.8085 | 0.0905 | 0.3285 | 0.8525 | 0.6065 | 0.7835 | 0.0555 | −0.3175 |
| AK2 | 1.7745 | 1.9995 | 2.8855 | 1.8565 | 0.1175 | 1.9755 | 1.8925 | 3.0505 | 2.6015 | 2.0955 |
| AK3L1 | 0.344 | 0.37 | −0.074 | −0.529 | −0.07 | 0.101 | −0.463 | −0.148 | −0.3 | −0.718 |
| AK5 | −0.048 | −0.38 | 0.347 | 0.218 | −0.363 | 0.034 | −0.453 | −0.159 | −0.171 | −0.058 |
| AKAP1 | −0.2235 | −0.0345 | −0.1855 | −0.4525 | 0.7885 | −0.0025 | 0.0025 | 1.4735 | 0.3235 | −0.3365 |
| AKR1A1 | −0.177 | −0.417 | 0.109 | 0.074 | 0.027 | 0.185 | −0.009 | 0.492 | 0.043 | −0.081 |
| AKR7A3 | −0.3105 | −0.6385 | 0.0575 | 0.0235 | −0.2045 | −0.6205 | 0.3315 | −0.1925 | 0.1205 | −0.2755 |
| ALG5 | −0.4685 | −0.3335 | 0.5295 | −0.3095 | 0.1895 | 1.0995 | 0.5565 | 0.6705 | −1.2595 | 0.4295 |
| ALG8 | 0.2395 | 0.0745 | −0.1315 | −0.2435 | −0.0165 | −0.6375 | −0.4365 | −0.5575 | 0.1145 | 0.2255 |
| AMFR | −0.908 | −0.77 | 0.007 | −0.493 | −0.223 | −0.442 | −0.281 | −0.292 | −0.357 | −0.409 |
| AMPD2 | −0.919 | −0.868 | −0.418 | 0.423 | −1.162 | −0.421 | −0.973 | −0.172 | −0.983 | −0.649 |
| ANAPC11 | −0.41 | −0.64 | 0.221 | −0.073 | −0.183 | −0.069 | 0.005 | −0.52 | −0.276 | −0.152 |
| ANKRD22 | −1.234 | −1.458 | −1.837 | −1.333 | −0.535 | −0.582 | −0.932 | −1.084 | −1.157 | −1.692 |
| ANKRD27 | 0.237 | −0.002 | 0.279 | 0.388 | −0.021 | 0.074 | 0.447 | 0.023 | 0.224 | 0.145 |
| ANLN | 0.759 | 0.487 | −0.056 | −0.024 | 0.605 | 0.321 | 0.78 | 1.247 | 0.953 | 0.261 |
| ANP32A | −0.68 | −0.736 | 0.912 | 0.946 | 0.407 | 0.29 | −0.015 | −0.127 | −0.55 | −0.401 |
| ANXA3 | −1.4905 | −0.7385 | −0.3905 | −1.1305 | −1.0245 | −0.1895 | −0.8945 | −0.3135 | −0.8955 | −0.6615 |
| APBB2 | 2.1685 | 3.1285 | −0.7305 | 1.1115 | −0.4085 | 1.0905 | 1.0395 | 2.0555 | 1.5735 | 0.6585 |
| APOBEC3 | −2.211 | −1.850 | −0.324 | 1.269 | 0.469 | −0.783 | 0.957 | 0.548 | −0.148 | −0.552 |
| AQP5 | 2.023 | 3.055 | −0.142 | −0.373 | 1.942 | −0.665 | 3.754 | 4.992 | 3.521 | 4.155 |
| AR | −1.3235 | −0.8005 | −0.2285 | −0.5545 | −0.5575 | 0.2775 | −0.8635 | −0.6325 | −0.3276 | −0.2345 |
| AREG | −0.296 | −0.546 | 0.26 | 0.691 | 0.342 | 0.49 | 0.585 | 0.203 | 0.593 | 0.523 |
| ARF1 | −0.8215 | −1.0725 | 0.5145 | −0.5385 | −0.1185 | −0.0145 | −0.3365 | −0.1785 | −0.1715 | −0.5475 |
| ARHGDI [symbol] | 0.909 | 0.223 | −0.001 | −0.327 | −0.288 | −0.557 | 0.02 | −0.159 | 0.258 | −0.101 |
| ARL6IP | 0.0055 | −0.5695 | 0.4255 | 1.1805 | 0.9045 | −0.3065 | 0.8516 | 0.6895 | 0.8705 | 0.3345 |
| ARL6IP2 | −0.784 | −0.393 | 0.701 | 0.911 | 1.210 | −0.046 | 0 | 0.123 | −0.076 | −0.735 |
| ARMCX2 | −0.907 | −0.569 | 0.737 | 0.096 | 1.272 | −0.095 | −0.191 | 0.181 | −0.166 | −0.589 |
| ARNT2 | 0.967 | 0.96 | 0.171 | −0.105 | 1.431 | 0.924 | −0.416 | −0.017 | 0.13 | −0.391 |
| ARNT2 | −0.7525 | −0.6585 | 0.1975 | −1.0555 | −0.0485 | 0.1065 | −0.5355 | −0.8875 | 0.0015 | −0.1335 |
| ARP3BET [symbol] | −0.5835 | −0.4535 | −0.0755 | −0.2795 | −0.8115 | −0.1285 | −0.6185 | −0.4115 | −0.1335 | −0.0405 |
| ARPC1B | 0.186 | 0.761 | 0.84 | −0.118 | 0.192 | 0.882 | 0.156 | −0.091 | −0.358 | 0.082 |
| ARPC5L | −0.1045 | −0.6105 | 0.1525 | 0.0535 | −0.6715 | −0.2965 | −0.0725 | 0.3945 | −0.4055 | −0.7935 |
| AS89 | −0.103 | −0.136 | −0.287 | 0.005 | 0.138 | 0.333 | −0.28 | −0.394 | −0.123 | 0.186 |
| ASF1A | −0.3575 | 0.8025 | −0.2975 | −0.5145 | −0.0705 | −0.4265 | −0.3385 | −0.5185 | −0.8375 | −0.7995 |
| ASH2L | −1.261 | −0.856 | −1.684 | −1.869 | −1.003 | −1.418 | −0.998 | −1.859 | −1.067 | −1.319 |
| ASK | 0.514 | 0.607 | −0.101 | 0.395 | 1.118 | 0.922 | −0.669 | −0.708 | −0.206 | −0.167 |
| ASPM | −0.4255 | −2.0755 | 0.2745 | −1.1785 | −1.3235 | −1.0925 | −0.6045 | −0.7465 | −1.3755 | −1.2055 |
| ASS | −0.4025 | 0.2105 | −0.4875 | −0.3685 | −0.2845 | 0.0545 | −0.5845 | −0.4585 | −0.2375 | 0.1545 |
| ATAD2 | 0.0185 | 0.7435 | 1.3445 | −0.0535 | 0.3915 | −0.0125 | 0.1215 | −0.1795 | −0.0585 | −0.0845 |
| ATF5 | 0.094 | 0.30 | 0.322 | −0.21 | −0.238 | 0.53 | 0.396 | 0.211 | −0.002 | −0.219 |
| ATF7IP2 | 0.096 | 0.159 | −0.117 | −0.359 | −0.096 | 0.158 | −0.334 | −0.045 | 0.067 | −0.218 |
| ATP2B1 | −0.256 | −0.123 | 0.121 | 0.377 | 0.146 | 0.107 | −0.03 | 0.261 | 0 | −0.072 |
| ATP5G1 | −0.8295 | −0.7465 | −0.1455 | −0.0395 | −0.2935 | −0.3525 | −0.3095 | −0.3845 | −0.2535 | −0.4165 |
| ATP5O | 0.785 | 1.313 | 2.283 | −1.722 | −1.703 | −0.526 | −1.224 | 0.315 | −0.811 | −0.576 |
| ATP6AP1 | −0.1 | 0.524 | −0.565 | 0.235 | 0.218 | 0.634 | −0.646 | 0.803 | 0.477 | 0.113 |
| ATP6V0A4 | −0.202 | −0.632 | −0.045 | 0.639 | −0.268 | 0.18 | −0.108 | 0.32 | −0.151 | −0.464 |
| ATP6V1B [symbol] | −0.5285 | −0.7095 | −0.7245 | 0.1305 | −0.6155 | 0.0945 | −0.4635 | 0.0905 | −0.2495 | −0.1995 |
| ATP6V1G [symbol] | 0.127 | −0.375 | 0.2 | −0.514 | −0.751 | −0.052 | 0.129 | 0.364 | 0.373 | 0.194 |
| ATPIF1 | 0.5975 | 1.7725 | 0.0035 | −0.0495 | 0.3795 | 0.5835 | 0.1485 | −0.0465 | −0.0035 | 0.1225 |
| ATXN10 | −0.019 | 0.283 | 2.245 | −0.387 | 0.103 | −0.107 | −0.083 | −0.177 | −0.478 | 0.525 |
| B3GNT5 | −0.3405 | 0.2125 | −0.1805 | −0.3795 | 0.0075 | 0.3815 | −0.1295 | 0.2445 | 0.3325 | 0.3185 |
| BACE2 | −0.1815 | −0.1985 | −0.2085 | 0.2185 | 0.1065 | 0.1805 | −0.2715 | 0.1805 | −0.0785 | −0.1305 |
| BAG1 | −0.1525 | −0.6255 | 0.0725 | 0.5555 | 0.1935 | 0.1365 | −0.9395 | 0.3355 | 0.2845 | 0.0435 |
| BAG4 | −0.2195 | −0.2685 | −0.0065 | 1.9275 | 1.7625 | 1.8665 | 0.9035 | −0.4575 | 2.1995 | 0.8705 |
| BAI2 | −0.281 | 0.32 | 0.835 | 0.254 | −0.104 | 1.365 | 1.013 | 0.052 | −0.799 | 0.057 |
| BAMBI | −0.014 | −0.039 | 0.738 | −0.681 | 0.258 | 0.004 | −0.045 | 0.084 | 0.531 | 0.074 |
| BCAS1 | −2.484 | −2.412 | 3.154 | 1.207 | 0.513 | 0.861 | 1.657 | 2.271 | 0.814 | 1.025 |
| BCL2L14 | 1.0185 | 0.9625 | −0.9455 | −0.0325 | −1.0865 | −0.0935 | 0.7835 | 3.5365 | 0.4185 | 0.0075 |
| BCMP11 | 1.322 | 1.449 | −0.924 | −1.043 | −2.266 | −0.398 | 0.03 | 1.4 | 1.596 | −0.007 |
| BEX1 | −1.8325 | −1.0045 | 2.7855 | −0.8516 | 1.7065 | 1.2445 | 1.4875 | 0.6535 | 1.9605 | 1.2105 |
| BEX2 | 0.2035 | −0.1816 | 0.0965 | −0.0985 | −0.1225 | −0.2175 | 0.2075 | −0.2605 | −0.0865 | 0.0165 |
| BF | −0.553 | −0.377 | 0.066 | −0.122 | −0.357 | −0.293 | 0.827 | 0.464 | −0.183 | −0.341 |
| BFSP1 | 0.267 | 0.085 | 0.374 | −0.03 | −0.205 | 0.362 | 0.047 | 0.01 | 0.234 | 0.202 |
| BIK | −1.996 | −1.361 | −1.324 | −2.592 | −2.003 | −1.488 | −1.456 | −1.629 | −1.42 | −2.269 |
| BIRC2 | −0.0685 | 0.0955 | 0.2735 | −0.9225 | −0.5695 | −0.0815 | −0.4965 | −0.3695 | −0.1125 | −0.1155 |
| BIRC5 | 0.6735 | −0.6425 | 0.1165 | 0.3205 | 0.4655 | 0.5225 | 0.4195 | 0.3075 | 0.2685 | −0.0225 |
| Bit1 | −0.3985 | −0.5885 | 0.6145 | −0.0085 | 0.4015 | −0.2745 | 0.0085 | 0.6535 | −0.8415 | −0.2265 |
| BIVM | −0.522 | 0.645 | −0.842 | −0.932 | −1.374 | −0.505 | −0.877 | −0.734 | −0.838 | −0.645 |
| BLVRA | 0.87 | 0.295 | −0.489 | −0.03 | 1.201 | −0.797 | −1.265 | 0.738 | −0.049 | 0.03 |
| BM039 | 0.678 | 1.008 | 0.786 | 1.14 | 1.297 | 0.528 | 0.902 | 1.358 | 0.315 | 0.112 |
| BMP7 | −0.214 | 0.158 | 0.106 | −1.385 | −0.911 | −0.591 | −0.412 | −0.564 | −0.234 | −1.054 |
| BOC | −0.1325 | −0.0575 | −0.0285 | 0.9515 | −0.3745 | 0.3755 | −0.1915 | −0.2995 | −0.1615 | −0.3725 |
| BRCA1 | −0.6095 | 0.0115 | 0.0835 | −0.0475 | 0.0505 | 0.0275 | −0.3755 | −0.5785 | −0.2545 | 0.0245 |
| BRF2 | −1.138 | 0.378 | −0.65 | −0.889 | −0.667 | −0.433 | −0.235 | −0.935 | −0.814 | −0.949 |
| BRI3 | −0.27 | −0.652 | 0.258 | 1.097 | −0.443 | −0.553 | 0.1 | 0.853 | 0.441 | −0.937 |
| BRI3BP | 0.374 | 0.145 | 0.848 | 0.096 | 0.134 | 0.117 | 0.211 | 0.594 | 0.274 | −0.027 |
| BSPRY | −0.0045 | 0.2235 | −0.0625 | −0.8505 | −0.5045 | 0.2395 | −1.0375 | 0.4015 | 0.4025 | 0.9665 |
| BTF3 | −0.0635 | −0.8585 | 0.5475 | −0.5095 | −0.8925 | 0.1475 | −0.2785 | −0.5085 | 0.0645 | 0.0385 |
| BTG3 | −0.752 | −0.437 | −1.023 | −1.002 | −1.243 | −1.234 | −0.987 | −1.555 | −1.178 | −0.98 |
| BγN2A2 | −0.739 | −0.572 | −0.427 | 0.409 | −0.128 | −0.839 | 0.381 | 0.501 | −0.153 | −0.332 |
| BUB1 | 0.0715 | 0.5285 | 0.3695 | 0.0785 | 0.0655 | 0.0025 | −0.0665 | 0.3675 | −0.2625 | 0.1885 |
| BUB3 | −0.318 | 0.03 | 0 | −0.179 | −0.308 | 0.514 | −0.167 | −0.4 | −0.148 | −0.214 |
| BXDC1 | −0.697 | −0.858 | −0.394 | −0.287 | −0.418 | −0.305 | 0.095 | 0.425 | −0.004 | 0.196 |
| BZRP | −0.347 | 0.984 | 0.382 | 1.753 | 1.551 | 0.702 | 0.877 | 0.483 | −0.68 | 0.558 |
| BZW1 | 0.2685 | 0.2465 | 0.0485 | −0.1875 | −0.2625 | −0.1795 | −0.2715 | −0.5125 | −0.3335 | 0.0635 |
| C10orf116 | −0.1175 | −0.0795 | 0.4795 | 0.2895 | 0.3085 | −0.0755 | −0.0105 | −0.1235 | 0.3555 | 0.1085 |
| C10orf38 | −0.952 | −0.148 | −0.284 | 0.081 | 0.326 | 1.785 | 0.181 | −0.093 | 0.148 | 0.107 |
| C10orf4 | −0.5175 | −0.4505 | −0.1485 | 0.0025 | −0.2255 | −0.3825 | −0.3895 | −0.2615 | −0.3195 | −0.2665 |
| C10orf45 | −0.2425 | −0.0905 | 2.7825 | −0.0985 | 0.9785 | 0.7095 | 0.9685 | 0.0005 | −0.6375 | 0.4955 |
| C10orf7 | −0.7075 | −0.8405 | −0.1785 | 0.2875 | 0.3955 | −0.2285 | −0.4275 | −0.0745 | −0.0405 | 0.8275 |
| C10orf82 | 0.003 | 0.889 | 0.091 | −0.482 | −1.123 | −0.814 | −0.096 | 0.177 | −0.251 | −0.215 |
| C11orf24 | −0.0595 | −0.8415 | 0.4825 | 0.7355 | 0.3665 | −0.3155 | 0.9365 | 0.8915 | 1.3575 | 0.5865 |
| C12orf14 | −0.788 | −0.866 | −0.548 | −0.265 | −0.851 | −0.957 | −0.718 | −0.623 | −0.426 | −0.324 |
| C13orf21 | 0.575 | 0.402 | 0.184 | −0.423 | −0.326 | 0.182 | 0.422 | −0.085 | 0.085 | 0.3 |
| C13orf23 | −0.695 | −0.465 | −1.088 | −0.667 | −0.481 | −0.646 | −0.76 | −0.874 | −0.104 | −1.24 |
| C14orf94 | 0.057 | 0.425 | 0.765 | −0.33 | 1.01 | −0.07 | 1.14 | 0.39 | 1.491 | 0.48 |
| C15orf23 | −0.5255 | −0.4495 | −0.0035 | −0.6355 | −0.3925 | −0.6585 | −0.4055 | −0.5185 | −0.5465 | −0.0415 |
| C16orf45 | −0.403 | −1.009 | −0.228 | −0.788 | −0.422 | 0.214 | 0.89 | −0.568 | −0.253 | 0.172 |
| C17orf37 | −0.298 | −0.364 | 0.485 | −0.17 | −0.043 | −0.222 | −0.215 | −0.147 | −0.338 | −0.027 |
| C18orf1 | −0.646 | −0.133 | −0.556 | 0.876 | 0.811 | 0.93 | 0.118 | 0.526 | 0.591 | 0.221 |
| C19orf10 | 0.2035 | 0.2855 | 0.8726 | −0.4716 | −0.2345 | 0.2515 | −1.1505 | 0.9555 | −1.1775 | −0.9516 |
| C19orf32 | −0.3185 | 0.2325 | 0.9695 | 1.4175 | 0.8745 | 1.3655 | 0.9445 | 1.5235 | 0.3425 | 0.2855 |
| C19orf33 | −1.108 | −1.591 | −0.389 | 0.695 | 0.106 | −0.704 | 0.578 | 1.6 | 0.738 | −0.042 |
| C1orf24 | −1.204 | −2.06 | 0.363 | 1.698 | 0.268 | 0.582 | 0.029 | −0.36 | 1.453 | 1.094 |
| C1orf34 | −1.2165 | −0.1805 | −0.3755 | 0.0025 | −0.0275 | −0.7175 | −0.3776 | −0.1995 | −0.5735 | −0.4445 |
| C20orf103 | −0.6405 | −1.2295 | 0.3025 | 0.6395 | −0.4475 | −0.1655 | 0.2055 | 0.5925 | 0.2005 | 0.1115 |
| C20orf149 | −0.555 | −0.835 | −0.594 | 0.814 | 0.214 | 0.448 | −0.261 | −0.266 | 0.086 | −0.187 |
| C20orf23 | −0.194 | −0.005 | 0.365 | −0.352 | 0.313 | 0.061 | 0.421 | 0.235 | 0.185 | −0.193 |
| C20orf3 | 0.138 | 0.281 | 0.311 | −0.28 | −0.013 | −0.022 | 0.214 | 0.133 | 0.12 | −0.126 |
| C20orf35 | 0.376 | 0.854 | 0.491 | 0.112 | −0.167 | 0.487 | 0.133 | 0.015 | −0.416 | −0.442 |
| C20orf43 | 0.2575 | 0.1355 | −0.5095 | −0.1295 | −0.1815 | −0.3985 | 0.0225 | −0.3305 | 0.0335 | −0.0535 |
| C20orf8 | −0.96 | −0.969 | 0.294 | −1.189 | −1.491 | −0.917 | −0.562 | −0.897 | −0.733 | −0.96 |
| C21orf45 | −0.055 | 0.172 | 0.228 | −0.527 | −0.628 | 0.187 | −0.592 | −0.384 | −0.177 | 0.017 |
| C22orf18 | −0.345 | 0.322 | −0.146 | −0.446 | −0.455 | 0.301 | −0.482 | 0.121 | 0.048 | 0.032 |
| C2F | −0.513 | −0.723 | 1.643 | 1.426 | 0.355 | 0.002 | 0.488 | 0.492 | 1.528 | 0.784 |
| C2orf17 | −0.612 | −1.257 | 0.282 | −0.139 | −0.726 | −0.413 | −0.742 | −0.705 | 0.781 | 0.18 |
| C4A | 0.095 | −0.046 | 0.254 | −0.612 | 0.083 | −0.796 | −0.228 | −0.264 | −0.054 | −0.311 |
| C5orf13 | −0.597 | 0.312 | 0.289 | 0.165 | −0.161 | −0.171 | −0.612 | −0.265 | −0.542 | −0.049 |
| C6orf108 | 0.287 | −0.06 | 0.392 | −0.338 | −0.647 | −0.407 | 0.192 | 0.286 | 0.075 | 0.221 |
| C6orf166 | −0.367 | −1.751 | 0.152 | 0.214 | 0.001 | −0.141 | 0.865 | 1.235 | 0.92 | −0.555 |
| C6orf192 | −0.1685 | −0.0495 | −0.1795 | −0.2575 | −0.4645 | −0.2805 | −0.3175 | −0.3395 | 0.0075 | −0.3915 |
| C6orf211 | −0.482 | 0.505 | 0.721 | 0.788 | 0.365 | 1.027 | 0.442 | −0.349 | 0.118 | −0.057 |
| C6orf49 | −0.5565 | −1.6025 | 0.2665 | −0.1305 | −0.5995 | 0.0175 | −0.1895 | 0.2415 | 0.2055 | −0.0345 |
| C6orf85 | −0.7775 | −0.5905 | 0.1285 | 0.7085 | 0.9285 | −0.4625 | 0.6885 | −0.0055 | −0.4395 | −0.5075 |
| C7orf24 | 0.837 | 0.352 | 1.942 | −1.206 | 1.682 | 0.189 | 1.185 | 1.478 | 0.384 | 0.455 |
| C9orf10 | 1.252 | 1.835 | 0.064 | −0.001 | 0.126 | 0.052 | −0.739 | 0.095 | −1.474 | −0.828 |
| C9orf121 | −0.186 | 0.216 | −0.244 | −0.001 | 0.243 | 0.077 | −0.565 | −0.139 | −0.33 | −0.529 |
| C9orf58 | −0.1795 | 0.4325 | 0.5185 | 0.7505 | 0.5985 | 0.3575 | −0.2095 | 0.4685 | 0.1405 | −0.0315 |
| C9orf88 | −1.431 | −0.471 | 1.426 | 1.088 | 1.935 | 0.755 | 0.439 | 0.988 | 0.365 | −0.462 |
| C9orf9 | −0.02 | −0.452 | −1.499 | 0.439 | −0.436 | −0.274 | 1.157 | 0.639 | −0.15 | 0.285 |
| CA12 | 0.254 | 0.468 | −1.501 | 0.389 | −0.472 | 0.048 | 0.977 | 0.902 | 0.058 | 0.005 |
| CA8 | 0.047 | 0.766 | 0.319 | 0.85 | 0.396 | −0.484 | 0.109 | 1.285 | 0.022 | 0.27 |
| CA8 | −0.1605 | 0.2495 | −0.0165 | −0.4225 | −0.2065 | 0.1205 | −0.1945 | −0.1615 | −0.0215 | 0.3255 |
| CACNA1G | 0.068 | −0.031 | 0.785 | 1.139 | 0.175 | 0.082 | −0.292 | −0.461 | 0.029 | 0.524 |
| CALCA | 0.742 | 0.685 | −0.906 | −0.296 | 3.097 | −0.738 | −0.896 | 0.37 | −0.34 | −0.26 |
| CALML4 | −0.8255 | −0.0655 | 0.2085 | 1.0335 | 0.2675 | 1.0735 | 0.2715 | 1.1735 | 0.3615 | 0.4705 |
| CALML5 | −0.6095 | −0.3755 | −1.2345 | 0.3075 | 0.7025 | −0.0305 | 0.8315 | 0.6935 | 0.3565 | 0.0065 |
| CaMKIINa [symbol] | −0.371 | 0.21 | −0.308 | 0.292 | 0.001 | 0.596 | 0.592 | −0.317 | −0.018 | −0.432 |
| CAP2 | −1.187 | −1.09 | 0.268 | −0.213 | −0.331 | 0.484 | 0.279 | 0.997 | 0.871 | −0.171 |
| CAP350 | 2.0575 | 1.2545 | 1.0835 | −0.9335 | −0.0645 | 1.8075 | 1.3015 | 4.0785 | −0.3405 | 0.4185 |
| CAPN13 | −0.709 | −1.032 | −0.278 | −0.578 | 0.029 | −0.669 | −0.037 | 0.08 | −0.146 | −0.221 |
| CAPN9 | −0.3045 | 0.0175 | −0.1355 | −0.1435 | −0.0085 | −0.2125 | 0.0275 | 0.5135 | −0.1835 | −0.0955 |
| CARD14 | 0.3825 | −0.3725 | 0.4885 | −0.4206 | −0.2625 | 0.9685 | −0.1265 | 0.9105 | 0.2825 | 0.2885 |
| CART | −0.8275 | −0.8195 | −0.2255 | 0.4225 | −0.3335 | −0.4835 | −0.1645 | −0.3065 | −0.6305 | −0.5135 |
| CASP1 | −0.527 | −0.51 | 1.038 | −0.142 | −0.399 | 0.797 | 0.058 | 0.083 | −0.262 | −0.182 |
| CASP3 | −0.168 | −0.443 | −0.077 | 2.229 | 0.906 | 1.881 | −0.368 | −0.06 | 1.547 | 0.816 |
| CASP7 | −0.0135 | 0.0135 | 1.2465 | 0.3945 | 0.0345 | −0.2855 | −0.6355 | −0.1855 | −0.0505 | −0.0355 |
| C9LN1 | −0.087 | 0.851 | −0.42 | 0.675 | 0.539 | 0.118 | −0.432 | 0.514 | 0.393 | 0.628 |
| CBR1 | −0.4605 | −0.8135 | 0.8385 | −0.0455 | −0.5015 | −0.0005 | 0.2675 | 0.4315 | 0.1845 | −0.0575 |
| CBR3 | 0.177 | 0.471 | −0.192 | 0.176 | 0.381 | −0.419 | 0.062 | −0.177 | 0.099 | 0.356 |
| CBR4 | 2.2355 | 2.8825 | 1.3525 | 0.4435 | −0.1935 | 0.3485 | 0.8415 | 1.2115 | −0.4355 | −1.2235 |
| CCL24 | −0.879 | −0.88 | −1.949 | −1.542 | −1.198 | −1.113 | −1.062 | −1.25 | −0.815 | −0.961 |
| CCL29 | −0.207 | −0.724 | 0.072 | −0.614 | −0.216 | 0.158 | 0.27 | 0.139 | −0.074 | −0.752 |
| CCNB2 | −0.1075 | −0.5016 | −0.8775 | −0.3855 | −0.4245 | −0.8535 | −0.4845 | −0.7705 | −0.4765 | −0.1285 |
| CCND1 | −0.055 | −0.988 | 1.277 | 1.709 | 0.692 | 0.432 | 2.095 | 1.805 | 0.926 | 0.324 |
| CCNE1 | −0.5075 | 0.2965 | 0.1395 | 0.1235 | 0.2795 | 0.7455 | 0.0965 | 0.4015 | 0.0785 | 0.2555 |
| CCNG2 | 0.48 | 0.007 | 0.521 | −0.323 | −0.754 | 1.236 | −0.347 | −0.288 | −0.87 | 0.454 |
| CCNH | −0.0115 | 0.8245 | −0.3475 | 0.0575 | −0.5835 | 0.5935 | 0.5115 | −0.5155 | −0.0425 | 0.6835 |
| CD38 | 0.0835 | −0.1115 | 0.4195 | 0.6695 | −0.0055 | −0.4905 | 0.0845 | −0.4255 | 0.1005 | 0.7425 |
| C030 | 0.0055 | −0.0785 | 0.5865 | −0.0425 | −0.0055 | 0.3365 | 0.2205 | 0.3945 | 0.1095 | −0.0925 |
| CD4 | 0.5925 | 1.2265 | 0.4575 | −0.4185 | −0.0126 | 0.6615 | 0.0405 | 0.8535 | 0.7615 | 0.5485 |
| CD58 | −1.078 | −0.822 | −0.791 | −1.017 | −1.053 | −0.859 | −0.526 | −0.701 | −0.78 | −1.083 |
| CD50 | −0.4855 | 1.8845 | −0.2385 | −1.1015 | −1.4225 | −0.5255 | −0.7785 | −0.9565 | −0.8035 | −1.0315 |
| CDC2 | −1.3195 | 0.7005 | −1.5205 | −1.8345 | −1.0585 | −1.0915 | −1.1125 | −0.8745 | −0.7485 | −1.5805 |
| CDC6 | −1.6635 | −1.6105 | −0.9265 | −1.9815 | −1.4765 | −1.7495 | −2.2035 | −1.4935 | −1.6115 | −1.8575 |
| CDCA1 | 0.258 | −0.888 | −0.523 | −0.718 | −0.959 | −0.225 | −0.808 | −1.026 | 0.098 | −0.457 |
| CDCA5 | −0.432 | −0.34 | −2.042 | −1.628 | −1.845 | −2.31 | −2.782 | 0.118 | −0.945 | −2.882 |
| CDCA7 | 0.4015 | 0.8615 | 0.5735 | −0.1775 | −0.5955 | −0.1475 | 0.2005 | 1.7145 | 0.9695 | 0.3885 |
| CDH1 | −0.458 | 0.189 | −0.082 | −0.625 | −0.406 | −0.452 | −0.651 | −0.515 | −0.57 | −0.298 |
| CDH3 | −0.2285 | −0.5895 | −0.6115 | 0.2515 | −0.1885 | −0.8185 | −0.3535 | −0.6295 | −0.1035 | 0.5345 |
| CDK2 | −1.1335 | −1.1555 | −0.2475 | −0.8305 | −0.7045 | −0.3335 | −0.5445 | −1.2395 | −0.5125 | −0.1565 |
| CDKN2A | −0.9085 | −0.1175 | −0.1205 | −1.1725 | −0.6925 | −0.9085 | −0.4435 | −0.8315 | −0.4595 | −0.8615 |
| CDKN2D | −0.227 | −0.512 | −0.823 | −1.093 | −1.638 | −0.269 | −1.076 | −0.857 | −0.614 | −0.95 |
| CDKN3 | −0.226 | −0.124 | −0.185 | −0.188 | −0.249 | 0.222 | −0.3 | −0.28 | −0.416 | −0.227 |
| CDT1 | −1.3835 | −1.4995 | 0.2935 | −0.5625 | −0.3095 | −0.2785 | 1.4635 | 0.8895 | 0.7975 | 0.0705 |
| CE9PG | 0.021 | −0.829 | −1.243 | −1.015 | −0.818 | −0.539 | −0.734 | −1.154 | −0.446 | −0.255 |
| CELSR1 | 0.104 | −0.756 | −1.016 | −1.113 | −0.912 | −0.546 | −0.716 | −1.07 | −0.686 | −0.327 |
| CENPA | −1.661 | −2.05 | −1.898 | −1.504 | −0.987 | −1.118 | −0.722 | −1.547 | −0.834 | −1.505 |
| CENPE | 0.391 | 0.213 | 0.062 | 0.006 | −0.176 | −0.162 | −0.454 | −0.402 | −0.337 | −0.084 |
| CENPF | 0.1505 | 0.2365 | −0.1405 | 0.2575 | 0.5395 | −0.2135 | 0.1025 | 0.4105 | 0.0385 | 0.1585 |
| CENTG3 | 0.0045 | −0.0045 | 0.4875 | 0.0325 | 0.3815 | −0.1405 | 0.4185 | 0.5935 | 0.0575 | −0.5005 |
| CES2 | 0.907 | 0.398 | −0.467 | −0.585 | −0.263 | 1.255 | −0.491 | −0.392 | 0.816 | −0.833 |
| CETN3 | −0.3445 | −0.1025 | 0.2215 | −0.8905 | −0.9215 | 0.0285 | −0.6155 | −0.8255 | −0.1625 | −0.4265 |
| CGA | 0.2965 | −0.5885 | 0.1285 | −1.2785 | −0.8255 | −0.1725 | −0.2265 | −0.0655 | 0.2815 | 0.0825 |
| CGI-12 | −1.1945 | −1.3825 | 0.3635 | −0.9965 | 0.2935 | 0.1725 | −0.0735 | 0.8925 | 0.5035 | 0.0375 |
| CGI-49 | −0.598 | −0.284 | 0.723 | −0.492 | 0.755 | −0.61 | 0.536 | −0.394 | 0.525 | −1.133 |
| CGI-4 [symbol] | 0.792 | 1.215 | 0.154 | 0.408 | 0.627 | −0.37 | 0.013 | 1.677 | 1.759 | 0.81 |
| CGN | −0.107 | 0.213 | −0.407 | −0.759 | −1.187 | −0.585 | −0.266 | −0.304 | −0.347 | −0.742 |
| CGNL1 | −1.3695 | −0.0755 | −0.3795 | −0.6825 | −0.7795 | −0.7705 | −1.1845 | −0.8425 | −0.8165 | −0.9735 |
| CHAF1B | −0.473 | −0.282 | −0.028 | −0.269 | −0.122 | 0.228 | −0.514 | −0.725 | −0.482 | −0.452 |
| CHC1 | −0.745 | −0.28 | 0.167 | 0.082 | −0.022 | −0.016 | 0.007 | 0.243 | 0.039 | −0.325 |
| CHCHD2 | −0.16 | −0.037 | 1.601 | −0.208 | −0.495 | −0.725 | −0.117 | 0.964 | 1.332 | 1.377 |
| CHCH05 | 1.353 | 0.448 | −0.228 | 0.38 | −0.568 | 0.814 | −0.216 | −0.022 | 2.542 | −0.361 |
| CHI3L1 | 0.814 | 0.83 | −0.428 | −0.127 | −0.879 | −0.284 | −0.329 | −0.729 | −0.596 | −0.47 |
| CHI3L2 | −0.185 | −0.508 | −0.028 | −0.108 | 0.095 | −0.4 | 0.003 | −0.044 | −0.079 | 0.217 |
| CHKA | 0.39 | 0.205 | 0.411 | −0.021 | 0.002 | −0.343 | 0.204 | −0.109 | 0.214 | 0.397 |
| CHCDL | −0.1455 | −0.2575 | −0.2365 | 0.2765 | 0.2695 | −0.4165 | 0.0415 | −0.2665 | 0.1275 | 0.5765 |
| CHRDL2 | −0.249 | −0.327 | 0.999 | 0.097 | 0.934 | −0.097 | 0.811 | 0.456 | 0.295 | 0.081 |
| CHST2 | −0.1365 | 0.4225 | 0.1855 | 0.1985 | −0.0835 | 0.0275 | 0.4945 | 0.2555 | 0.1435 | 0.1465 |
| CHURC1 | 1.007 | 1.772 | 0.556 | 0.314 | 0.22 | 1.435 | 0.656 | 1.142 | 1.219 | 0.881 |
| CIR | −0.5585 | −0.8065 | −0.2315 | −0.0355 | −0.1185 | −0.5875 | −0.1935 | −0.2065 | −0.0575 | −0.4425 |
| CIRBP | −0.1945 | −0.4245 | 0.3725 | −0.0495 | −0.2075 | −0.2485 | 0.3875 | 0.4835 | 0.1575 | 0.0005 |
| CKLF | 1.1505 | 2.1595 | 0.9905 | −0.1515 | −0.0965 | −0.1285 | −0.0245 | 0.5635 | −0.3315 | 0.1065 |
| CKLFSF6 | 0.0385 | 0.3975 | −1.9095 | −0.1705 | −0.8215 | −0.8165 | −1.4485 | −0.2335 | −0.5085 | −1.0205 |
| CKLFSF7 | −0.35 | −0.409 | −0.22 | 0.276 | 0.371 | −0.433 | 0.036 | −0.429 | 0.044 | −0.541 |
| CKMT1 | −0.847 | −1.406 | −0.387 | −0.491 | −0.287 | −1.262 | −0.376 | −1.443 | −1.667 | −1.131 |
| CKS1B | −0.379 | −0.975 | 0.122 | −0.491 | −0.545 | −0.035 | −0.578 | −0.092 | −0.362 | −0.21 |
| CKS2 | 0.0115 | −0.4125 | −0.0255 | 0.0055 | 0.1585 | 0.2785 | −0.1985 | −0.1855 | −0.1395 | −0.6715 |
| CL640 | −0.4535 | 0.7505 | −0.6645 | −1.0985 | 0.7545 | −0.5245 | −0.8435 | −0.0625 | −0.8835 | −0.7925 |
| CLDN23 | −0.4725 | 1.1995 | 0.3685 | −0.5735 | 0.9755 | −0.5125 | −0.9265 | −0.6195 | −0.8325 | 0.2225 |
| CLDN3 | 0.8095 | 0.2005 | 1.4915 | 1.7405 | −0.5835 | −0.3655 | 1.0495 | 1.9985 | −0.5835 | −0.1715 |
| CLDN4 | −0.628 | 1.893 | 2.612 | 2.376 | 1.382 | 1.803 | 3.432 | 1.312 | 1.392 | 0.189 |
| CLDN8 | 0.0785 | 0.3785 | 0.2075 | 0.0925 | 0.5915 | 0.2515 | 1.5065 | 3.4925 | 1.6095 | 0.1055 |
| CLGN | −0.0405 | −0.3995 | 0.0255 | −0.2775 | −0.1835 | −0.5115 | 0.1095 | −0.2135 | 0.0945 | −0.3305 |
| CLIC6 | −0.846 | −1.161 | 0.053 | −0.262 | −0.283 | 0.149 | −0.128 | −0.217 | −0.019 | −0.068 |
| CLNS1A | 1.3345 | 1.4865 | 1.7865 | 1.2185 | 1.3285 | 1.9505 | 0.6345 | 0.6285 | 1.2885 | 0.2115 |
| CLTC | −0.019 | −0.149 | −0.018 | −0.060 | −0.129 | −0.518 | −0.112 | 0.398 | 0 | −0.46 |
| CLU | −0.09 | −0.3 | 0.81 | −0.546 | −0.189 | −0.019 | −0.454 | −0.512 | −0.48 | −0.559 |
| CMAS | −0.3795 | −0.8895 | 0.6095 | 0.3115 | 0.1055 | −0.2125 | −0.0175 | 0.3615 | −0.1645 | −0.3765 |
| CML65 | −0.228 | 0.503 | −0.42 | 0.011 | −0.241 | −0.554 | −0.142 | −0.318 | 0.113 | −0.505 |
| CNIH | −0.301 | 0.147 | −0.157 | 0.282 | −0.227 | 0.156 | −0.067 | −0.229 | −0.109 | −0.048 |
| CNOT10 | 0.2985 | 0.1305 | −0.4365 | 0.0725 | −0.2745 | −0.1415 | 0.0425 | 0.1485 | 0.3105 | 0.0595 |
| CNOT2 | 0.002 | −0.054 | −0.003 | −0.594 | 0.123 | −0.185 | −0.392 | 0.087 | −0.368 | 0.027 |
| CNOT7 | −0.989 | 0.517 | −1.876 | −1.597 | −2.011 | −0.988 | −0.286 | −0.103 | 0.651 | −1.988 |
| CNP | 0.2845 | 0.1255 | 0.1815 | −0.4145 | 0.1355 | −0.2095 | −0.4065 | −0.1335 | 0.2215 | −0.3075 |
| CNTNAP2 | 0.389 | −0.002 | −0.098 | 0.29 | 0.174 | 0.196 | 0.332 | 0.166 | −0.113 | −0.03 |
| COASY | 0.9045 | 0.3305 | −0.7385 | −1.2525 | 0.8605 | 0.6585 | −0.2575 | −0.7075 | 0.3825 | 0.1225 |
| COL11A2 | 0.272 | −0.356 | 0.078 | 0.862 | −0.751 | 0.098 | 0.856 | 1.175 | 2.258 | 0.818 |
| COL27A1 | 0.446 | −0.22 | −0.422 | −0.44 | −0.575 | −0.384 | 0.106 | 0.11 | −1.083 | 0.455 |
| COL4A5 | 0.7285 | 0.6505 | 0.3665 | −0.5955 | −1.7145 | −0.3875 | −0.0165 | 0.3085 | 0.3365 | 0.5065 |
| COL8A1 | −0.206 | −0.651 | 0.18 | 0.344 | −0.168 | 0.25 | 0.302 | 0.012 | 0.563 | 0.175 |
| COL8A3 | −0.664 | −0.73 | 0.37 | −0.638 | −0.308 | −0.383 | −0.322 | −0.31 | −0.23 | −0.1 |
| COMMD1 [symbol] | 0.1705 | 0.0135 | 0.0535 | −0.4105 | −0.2205 | −0.1995 | −0.2445 | −0.2485 | −0.2095 | 0.0225 |
| COMMD5 | 0.0355 | 0.3565 | 2.1285 | −0.0335 | 0.0605 | −0.1625 | 0.0455 | −0.2605 | 0.7695 | 0.3315 |
| COPS7B | −0.055 | 0.112 | −0.458 | 0.05 | −0.361 | −0.563 | −0.006 | −0.36 | −0.684 | 0.086 |
| CORO6 | −1.1855 | −0.8755 | 0.1775 | 0.0695 | −0.3115 | 0.0565 | −0.1985 | −0.3075 | −0.1905 | −0.4005 |
| CORT | −0.319 | −0.356 | 0.357 | 0.094 | −0.247 | 0.268 | −0.429 | −0.244 | −0.298 | −0.402 |
| COX17 | −0.522 | −0.48 | 0.277 | 0.143 | −0.089 | −0.026 | −0.289 | −0.354 | −0.283 | −0.388 |
| COX5A | −0.789 | −0.815 | −0.628 | −0.313 | −0.019 | −0.625 | −0.012 | −0.537 | −0.544 | −0.54 |
| COX6A | −1.4725 | −1.1105 | −0.1605 | −0.4115 | −0.4055 | 0.2035 | −0.4165 | 0.8525 | 0.5885 | −1.0275 |
| COX8A1 | −0.7116 | −0.7005 | 0.1295 | 0.3145 | −0.1275 | −0.3375 | −0.3365 | −0.0935 | −0.7025 | −0.6315 |
| COX8C | 0.0685 | 0.4185 | 2.0745 | 0.9835 | −1.1525 | 0.1385 | −0.8235 | −0.0175 | −0.3035 | 0.0075 |
| COX7A2 | 0.721 | 1.08 | 1.41 | 0.246 | 0.833 | −0.197 | −0.331 | 0.239 | −0.196 | −0.245 |
| CP | −0.2445 | −0.8335 | 1.4655 | 0.4425 | 1.0075 | −0.0655 | −0.0255 | 1.2195 | 0.2565 | 1.6145 |
| CPAMD0 | 0.132 | −0.022 | 0.434 | 0.802 | −0.134 | 1.236 | 0.572 | −0.025 | 0.114 | −0.416 |
| CPB1 | −0.8965 | −1.1405 | 0.9475 | 0.3965 | 0.5535 | 0.6545 | 0.4715 | 0.4355 | 0.0095 | 0.2025 |
| CPEB3 | −0.463 | −0.121 | −0.236 | −0.165 | −0.623 | −0.565 | −0.101 | −0.207 | −0.262 | −0.314 |
| CPEB4 | 0.69 | 0.656 | −0.313 | 1.003 | −0.236 | −0.856 | 0.487 | 0.679 | −0.501 | 0.034 |
| CPSF3 | −0.8785 | −0.5505 | −0.2555 | 1.1875 | 0.8675 | 0.8505 | 0.7315 | 0.2065 | −0.0135 | 0.1085 |
| CRABP1 | −2.108 | −0.874 | −0.382 | −1.89 | 0.031 | 0.623 | 0.138 | −0.268 | 0.107 | 0.816 |
| CREB3L4 | 0.283 | 0.574 | 1.161 | −0.119 | −0.225 | −0.674 | 0.597 | −0.411 | −0.252 | 0.755 |
| CRIP1 | −0.018 | −0.142 | 0.379 | 0.532 | 0.735 | −0.01 | 0.844 | −0.293 | 0.633 | 0.409 |
| CRYBB2 | 0.23 | −0.447 | −0.182 | 0.215 | 0.798 | −0.345 | 0.698 | 0.754 | −0.751 | −0.088 |
| CRYL1 | 0.490 | 1.532 | −0.287 | −0.171 | 0.548 | 0.008 | −0.108 | −0.008 | 0.238 | 0.39 |
| CRYZ | −0.5805 | −0.3635 | −0.5855 | −0.4795 | −0.5445 | −0.4435 | −0.6565 | −0.3315 | 0.0225 | −0.2775 |
| CSDA | −0.0055 | 0.1665 | 0.1915 | −0.1025 | 0.1815 | −0.2175 | 0.0055 | −0.2055 | −0.0705 | 0.3585 |
| CSE1L | 1.4915 | 1.6795 | 0.1995 | 0.1085 | 0.3625 | 0.3805 | 0.0245 | 1.7215 | 1.3645 | 1.1735 |
| CSK | 0.2285 | 0.4185 | 1.1315 | −0.0165 | 0.0615 | 0.7875 | −0.0525 | −0.2075 | 0.4295 | 0.6805 |
| CSRP1 | −0.9325 | −1.5165 | −1.4115 | 0.5595 | 0.0035 | −0.2405 | 0.5605 | −0.4835 | 0.7455 | −0.0035 |
| CSRP2 | −0.011 | −0.319 | −0.372 | −0.384 | 0.059 | 0.024 | 0.043 | −0.595 | −0.272 | −0.267 |
| CST5 | 0.9805 | 0.8215 | −0.3185 | −1.3485 | −0.9275 | 2.7465 | −0.8255 | 1.1545 | −0.2895 | 0.0355 |
| CSTB | 0.077 | −0.048 | 0.79 | 0.611 | 0.239 | 0.22 | −0.197 | −0.183 | −0.001 | 0.17 |
| CTAG1B | −0.1585 | 0.0395 | −0.3145 | 0.1735 | −0.0155 | 0.4415 | 0.1865 | −0.0495 | −0.0975 | 0.0955 |
| CTBP2 | 0.37 | 0.265 | 1.422 | 0.295 | 0.344 | 0.466 | 0.199 | 0.976 | 0.329 | 0.815 |
| CTNNA1 | 0.019 | −0.074 | 0.276 | 0.435 | −0.495 | −0.576 | 0.924 | 0.41 | −0.745 | 0.001 |
| CTNNAL1 | −0.6035 | −0.5975 | −0.4495 | −0.9025 | −1.0895 | −0.7585 | −1.0515 | −0.9305 | −0.0595 | −0.1945 |
| CTNND2 | −0.469 | 0.441 | −1.243 | −0.645 | −1.858 | −1.205 | −1.169 | −0.744 | −0.575 | −0.284 |
| CTPS | −0.713 | −1.519 | 0.762 | −0.202 | −0.21 | 0.881 | 0.72 | −0.387 | 0.872 | 0.752 |
| CTSL2 | −0.0125 | −1.1135 | 0.1005 | −0.4655 | −0.6505 | 0.0055 | −0.5975 | −0.1475 | −0.2375 | −0.6345 |
| CTSS | 1.99 | 2.99 | 0.991 | 0.52 | −0.04 | 0.68 | 0.04 | 1.34 | 0.811 | 1.02 |
| CTTN | −2.5125 | −3.0475 | −1.7715 | 1.2085 | −0.9965 | 0.5405 | 1.0325 | 0.4885 | −2.4925 | 0.0015 |
| CX3CL1 | −1.4195 | −1.5505 | 0.0995 | 0.7585 | −0.3985 | −1.2455 | −0.0115 | −0.1235 | −0.1735 | −0.6705 |
| CXCL9 | −0.0655 | −0.1975 | 0.0615 | 0.9345 | 0.8605 | 0.4835 | 0.8075 | 0.1735 | −0.0145 | −0.0555 |
| CXorf39 | −1.017 | −1.337 | −0.725 | 0.251 | 0.17 | 0.902 | 0.38 | −0.006 | 1.181 | −0.003 |
| CXXC4 | −0.696 | −0.87 | 0.446 | −0.838 | −0.085 | −0.001 | −0.522 | −0.03 | −0.084 | −0.786 |
| CYB5 | −0.3215 | 0.4205 | −0.0655 | 0.1205 | −0.0805 | −0.2515 | −0.3595 | −0.3725 | −0.3325 | −0.5415 |
| CYB561 | −0.25 | −0.004 | 0.685 | −0.474 | 0.114 | 0.032 | −0.093 | 0.04 | −0.01 | −0.325 |
| CYC1 | −0.14 | 0.025 | −0.292 | −0.03 | 0.095 | −0.594 | −0.201 | −0.35 | −0.158 | −0.083 |
| CYHR1 | −1.0395 | −0.9265 | 1.1495 | 2.1075 | 2.1755 | 1.6755 | 1.8215 | 2.2145 | 2.7315 | 2.1715 |
| CYP2A13 | 0.222 | 0.402 | −0.215 | −0.822 | −0.688 | 0.445 | −0.528 | −0.397 | −0.227 | 0.131 |
| CYP4X1 | −0.9995 | −1.8305 | 0.3525 | 1.9535 | 0.8875 | 0.5785 | 2.4845 | 2.6035 | 1.0215 | −0.2205 |
| D15Wsu7 [symbol] | −1.0345 | −1.3995 | 0.6655 | 0.8515 | 0.1895 | 0.3515 | 1.1405 | 1.4615 | 0.7645 | −0.0505 |
| DACH1 | −1.177 | −1.495 | 0.55 | 2.385 | 1.203 | 0.126 | 2.458 | 2.859 | 1.479 | −0.257 |
| DACH1 | −0.729 | −0.929 | −0.374 | 0.254 | −0.155 | −0.219 | −0.365 | −0.404 | 0.319 | −0.136 |
| DACH1 | −0.073 | −0.541 | 0.045 | −0.589 | −0.151 | 0.642 | 0.169 | −0.001 | −0.079 | −0.498 |
| DAP3 | −0.2675 | −0.0435 | 0.5815 | 0.2975 | −0.1415 | 0.0015 | 0.2185 | 0.2205 | 0.3065 | 0.3195 |
| DAPP1 | 0.234 | 0.197 | −0.182 | 2.274 | −0.077 | 1.098 | −0.289 | 0.094 | −0.128 | −0.472 |
| DAZAP2 | −0.508 | −0.833 | 0.156 | −0.318 | −0.144 | −0.355 | −0.621 | −0.218 | 0.042 | 0.172 |
| DBI | −0.3105 | −0.4825 | −0.6315 | −0.6455 | −0.8155 | −0.5345 | −0.6205 | −0.6015 | −0.7595 | 0.0595 |
| D9N1 | −0.43 | 0.83 | 0.283 | −0.249 | −0.332 | −0.327 | −0.641 | −0.035 | −0.01 | −0.288 |
| DC13 | 0.0425 | −0.1015 | −0.3125 | −0.1305 | −0.0485 | 0.2485 | −0.4005 | −0.3005 | −0.1335 | −0.0205 |
| DCPS | −0.3225 | 0.1335 | 0.2685 | 0.1555 | −0.3115 | −0.1315 | 0.8515 | −0.0205 | −0.2625 | 1.2535 |
| DDHD2 | −0.5065 | −0.4845 | −0.4135 | −0.6195 | −0.6835 | −0.5995 | −0.4005 | −0.9505 | −0.5095 | −0.2905 |
| D0X17 | −0.2675 | 0.3075 | −0.3375 | −1.1565 | −0.4875 | −0.2255 | −0.2905 | −0.5135 | −0.4375 | −0.0975 |
| DDX39 | −1.161 | −1.475 | −1.061 | −0.340 | −1.46 | 0.329 | −1.11 | −0.981 | 0.164 | −0.489 |
| DDX48 | −0.2605 | −0.4875 | 0.3505 | −0.3215 | −0.8755 | −0.3385 | −0.4375 | −0.3705 | −0.4425 | −0.1375 |
| DDX58 | −0.1485 | −0.4535 | 0.0865 | −0.2445 | 0.3885 | 0.3555 | 0.4505 | 0.1465 | 0.1005 | −0.2455 |
| DEF6 | −0.001 | 0.018 | −0.946 | −0.997 | −1.247 | −0.889 | −0.899 | −1.458 | −0.382 | −0.341 |
| DEK | −0.386 | −0.693 | 1.031 | −0.395 | −0.068 | −0.18 | 0.295 | 0.625 | 0.073 | 0.188 |
| DHCR7 | 0.278 | 0.801 | −0.031 | 0.467 | 1.269 | 0.749 | −0.719 | −0.818 | −0.313 | −0.218 |
| DHRS7 | 0.2055 | 1.5925 | −1.4055 | 0.0035 | −0.0895 | 1.1255 | −0.5285 | 0.5755 | 1.5625 | 0.3705 |
| DHX57 | 0.1225 | −0.3085 | −0.6365 | −0.9425 | 0.1845 | 2.4855 | 1.0495 | 0.1445 | −1.5025 | 2.1455 |
| DIRC1 | 0.588 | 1.011 | −0.088 | −0.227 | −0.262 | 0.035 | −0.298 | −0.145 | −0.244 | −0.023 |
| dJ222E13 | 0.262 | −0.218 | −0.773 | −0.469 | −0.627 | 1.362 | −0.119 | −0.217 | 0.227 | 0.061 |
| DKFZP434 | −0.409 | 0.303 | 0.302 | −0.433 | −1.134 | −0.173 | −0.716 | 0.179 | −0.331 | −0.308 |
| DKFZp434 | 0.4935 | −0.3875 | −0.9035 | −0.9295 | −1.0565 | −0.1665 | −0.5985 | −1.1875 | −0.7385 | −0.6985 |
| DKFZP434 | −0.352 | −0.024 | 0.055 | 0.355 | 0.132 | 0.122 | 0.055 | 0.069 | 0.127 | 0.056 |
| DKFZp434 | −0.6075 | −0.3965 | 0.1216 | −0.1805 | −0.1655 | −0.5345 | 0.1455 | −0.2105 | −0.1025 | 0.0175 |
| DKFZP564 | −0.4325 | −0.4805 | 1.0435 | −0.1955 | −0.0855 | −0.1285 | −0.3705 | −0.2715 | 0.3095 | −0.7325 |
| DKFZp564 | −0.708 | −1.242 | 0.303 | −0.595 | −0.45 | −0.860 | −0.18 | −0.383 | −0.397 | −0.298 |
| DKFZP564 | −0.5615 | −0.0555 | 0.3445 | −0.0655 | −0.0015 | 0.1465 | 0.0425 | 0.4295 | 0.4985 | 0.0055 |
| DKFZP564 | −0.044 | −0.437 | 0.277 | 0.47 | 0.338 | 0.154 | −0.05 | 0.258 | −0.038 | 0 |
| DKFZP564 | 0.592 | 0.585 | 0.082 | −0.222 | 0.988 | 0.918 | 0.291 | 1.143 | 1.297 | 0.489 |
| DKFZp586 | 3.8585 | 4.0955 | −1.4175 | −0.0585 | −0.6405 | 0.1205 | 1.0555 | 4.4765 | 0.4545 | 0.5055 |
| DKFZp761 | −0.126 | −0.469 | −0.290 | −0.079 | −0.122 | −0.223 | −0.124 | −0.374 | 0.152 | 0.363 |
| DLK1 | −0.517 | −1.428 | 0.726 | −0.276 | −0.49 | 0.468 | −0.012 | −0.236 | −0.682 | −0.127 |
| DLX5 | −0.8065 | −0.5295 | 0.8635 | 0.5055 | 0.3135 | 0.7525 | −0.0295 | −0.0545 | 0.1795 | −0.4005 |
| DNAJA4 | −0.5255 | −0.5005 | 0.8515 | −1.3295 | −0.0725 | 0.9585 | 1.6905 | 1.4085 | 1.1695 | 0.0545 |
| DNAJC1 | −0.931 | −0.342 | −0.783 | −0.303 | −0.112 | −0.011 | 0.138 | 0.192 | −0.114 | −0.264 |
| DNAJC12 | −1.42 | −1.839 | 1.191 | 2.171 | 0.438 | 0.193 | 0.876 | 1.638 | 0.96 | 0.835 |
| DNAJD1 | −0.208 | −0.74 | −0.494 | −0.596 | −0.649 | −0.14 | −0.02 | −0.041 | −0.132 | −0.478 |
| DNALI1 | 0.332 | 0.574 | 1.628 | −0.307 | 0.556 | 0.99 | −0.079 | −0.738 | 0.153 | 0.228 |
| DNMT1 | 1.5275 | 1.5755 | 1.1185 | 0.0125 | 0.5425 | 0.3625 | 1.0455 | 2.5465 | 0.4645 | 1.6015 |
| DOX1 | −0.6575 | −1.1005 | 0.0005 | −0.3415 | −0.5405 | −0.1925 | −0.0285 | 0.2135 | −0.3505 | −0.2375 |
| DSCR1 | −0.5935 | −0.4365 | −0.5275 | 0.2755 | −0.4595 | −2.3745 | 0.3885 | 0.5305 | 0.8745 | −0.4985 |
| DSCR1L2 | −0.4295 | −0.6565 | −0.1785 | −0.7875 | −0.6095 | −0.3785 | −0.4125 | −0.6905 | −0.3135 | −0.6695 |
| DSP | −0.6405 | −0.0995 | 0.7625 | 0.2895 | 0.3955 | −0.1995 | −0.1615 | −0.1305 | 0.3325 | 0.0605 |
| DTYMK | −0.2945 | 0.4225 | −0.3105 | 0.7855 | 0.6015 | 1.0575 | 0.0785 | 0.4285 | 0.1895 | 1.1475 |
| DUSP23 | 1.0035 | 0.6405 | 0.8185 | 0.2175 | 0.5585 | 1.8425 | 0.2415 | 1.3995 | 0.5025 | 1.1035 |
| DUSP4 | 0.0555 | 0.1185 | −0.4655 | 0.1305 | 0.1915 | −0.2405 | −0.2395 | −0.1535 | 0.1365 | −0.2235 |
| DUSP5 | −0.068 | 0.08 | 0.144 | −0.496 | −0.307 | −0.142 | −0.337 | −0.057 | 0.333 | 0.132 |
| DVL1 | −0.076 | −0.311 | 0.097 | 0.501 | −0.134 | −0.954 | 0.13 | −0.022 | −0.193 | 0.113 |
| DXYS155 [symbol] | −0.38 | −0.624 | 0.358 | −0.532 | −0.512 | −0.301 | −0.537 | −0.782 | −0.726 | −0.515 |
| DYRK4 | 0.3005 | −0.3895 | 0.9755 | 0.9165 | −0.7375 | 1.0745 | 0.8265 | 0.3405 | −0.2785 | 0.0065 |
| o(y)2 | 0.018 | −0.22 | −0.086 | −0.435 | 0.208 | −0.212 | −0.105 | −0.191 | −0.077 | −0.03 |
| EAF2 | 0.4615 | 0.4915 | 0.4105 | 1.2295 | 0.8665 | 1.7205 | 0.8315 | 0.8975 | 0.2585 | −0.0675 |
| EAP30 | 0.1535 | 0.1695 | 0.3315 | 1.1995 | 1.225 | 0.5375 | −0.8495 | −0.0375 | −0.0445 | −2.2685 |
| ECHDC1 | 0.5545 | 1.5855 | −0.8735 | −0.6395 | −0.0785 | 0.3165 | −1.0685 | −0.3335 | 0.0295 | −0.0615 |
| ECHDC3 | −0.1265 | −1.0035 | 0.0825 | 0.3305 | 0.2565 | −0.8316 | 1.0515 | 0.8555 | 1.1075 | 0.0385 |
| EEF1A2 | 0 | 0.507 | 0.063 | 0.939 | −0.104 | −0.485 | 0 | −0.193 | −0.062 | −0.491 |
| EFHC1 | 0.0215 | −0.0335 | −0.8995 | −0.5385 | −0.6435 | 0.0425 | −0.0895 | 0.2545 | −0.2715 | −0.2035 |
| EFNA4 | −0.0355 | −0.1345 | −0.2885 | −0.1935 | −0.4205 | −0.5555 | −0.3205 | −0.4335 | −0.4145 | 0.1095 |
| EFS | −0.0875 | −0.5935 | 0.2235 | −0.2185 | 0.0695 | −0.1915 | 0.5005 | 0.4505 | 0.3795 | −0.1745 |
| EIF2C2 | −0.0305 | 0.4305 | −1.2205 | 0.0095 | −0.2675 | −0.5915 | −0.8715 | −1.1115 | −0.4375 | −0.2705 |
| EIF2C4 | −0.004 | 0.01 | −0.203 | −0.227 | 0.117 | −0.121 | −0.532 | −0.345 | 0.33 | −0.171 |
| EIF4EBP1 | −0.2875 | −0.5335 | −0.2435 | 0.1675 | 0.3615 | 0.5315 | 0.1085 | 0.1585 | 0.0115 | −0.3735 |
| EIF4G1 | 0.371 | 0.282 | 0.464 | −0.461 | −0.368 | 0.168 | −0.336 | 0.345 | 1.061 | 0.173 |
| EIF4G3 | −0.291 | −0.046 | 1.33 | 0.370 | −0.598 | 0.186 | −0.172 | −0.198 | −0.368 | 1.464 |
| ELAC1 | −0.116 | −0.687 | −1.354 | −0.382 | −0.773 | −0.286 | −0.044 | −0.168 | −0.761 | −0.815 |
| ELL2 | 0.0375 | −1.0785 | −0.0545 | 0.4845 | 0.4725 | 0.8245 | 1.1995 | 1.2365 | 0.7865 | 0.1965 |
| ELL3 | −0.3315 | 0.5385 | −0.8975 | −0.4975 | 0.5465 | 0.1685 | 0.1985 | −0.1835 | −0.0315 | 0.1425 |
| ELOVL5 | −0.7535 | −1.0625 | −0.5665 | 0.4525 | 0.1035 | 0.5895 | 0.5025 | 0.3675 | 0.9705 | 0.8735 |
| EN1 | 0.6305 | 0.5795 | 0.6735 | 0.7305 | 1.1365 | 1.7975 | 1.2275 | 1.0655 | 0.6415 | −0.1235 |
| ENAH | 0.0195 | −0.4255 | 1.0585 | 1.2345 | 1.0865 | 0.9205 | 1.0495 | 0.8125 | 0.7985 | 1.2215 |
| EPHX2 | −1.125 | −1.305 | 0.151 | −0.286 | −0.09 | −0.847 | −0.035 | 0.096 | −0.127 | −1.019 |
| EPLIN | 0.2065 | 0.3805 | 0.1225 | −0.6965 | 0.7025 | 0.1335 | 1.3105 | 0.4955 | 0.8275 | −0.2675 |
| EPN3 | −0.9905 | −0.2945 | −1.2775 | −0.1635 | −0.3895 | 0.4685 | 0.3225 | 0.5845 | 0.3115 | −0.0885 |
| EPOR | −0.779 | −0.285 | 0.754 | 0.044 | −0.412 | −0.397 | −0.523 | −0.296 | −0.42 | −0.408 |
| ERBB2 | −0.7795 | −1.6215 | 0.7395 | −0.0535 | 0.6465 | 0.5045 | 1.6105 | 1.3375 | 1.7015 | −0.3265 |
| ERP70 | −0.077 | −0.642 | 0.413 | 0.75 | 0.433 | 1.3 | −0.043 | 0.506 | −0.029 | −0.105 |
| ESR1 | −0.2425 | 0.1255 | 0.5995 | 0.1305 | −0.4515 | −0.3445 | −0.3855 | −0.3685 | −0.0465 | 0.1165 |
| ETFA | −0.1575 | −0.3885 | −0.0515 | −1.2005 | 0.1335 | 0.0555 | −0.0495 | −0.6175 | −0.2855 | −0.4695 |
| ETV8 | −0.3035 | 1.4685 | −1.2235 | −1.3505 | −0.9285 | −0.4695 | −0.6975 | −1.3435 | −0.2835 | −1.0035 |
| EVER1 | −0.4455 | 0.1885 | −0.1035 | −0.7745 | −0.4155 | 0.6155 | −0.4035 | −0.0225 | −0.0975 | −0.1965 |
| EXO1 | −0.2305 | −0.4925 | 0.3955 | −0.0055 | 0.7245 | −0.1695 | 0.0825 | 0.4465 | 0.4535 | 0.3805 |
| EXOSC9 | −0.667 | −1.279 | −1.161 | −1.16 | −1.096 | −1.128 | −0.524 | −1.14 | −0.708 | −0.834 |
| EXT2 | −0.008 | −0.702 | −0.734 | −0.042 | 0.456 | 0.224 | 0.721 | 0.559 | 0.852 | −0.376 |
| EZH2 | 0.0795 | −0.5115 | −0.4195 | 0.4835 | −0.2765 | −0.0955 | 0.0105 | 0.2725 | −0.2405 | 0.2505 |
| FI1R | 0.442 | 0.976 | −0.535 | −0.015 | −0.184 | 0.922 | −1.299 | 0.123 | −1.353 | −0.746 |
| F9 | 2.148 | 1.122 | 0.903 | −0.781 | −1.792 | 0.055 | 0.029 | 1.02 | 0.956 | 0.905 |
| FA2H | −0.25 | −0.333 | 0.093 | −0.206 | 0.612 | −0.63 | −0.468 | −0.02 | −0.122 | −0.304 |
| FABP7 | −0.911 | −0.888 | 1.068 | −0.337 | −0.288 | −0.181 | −0.246 | −0.223 | 0.056 | −0.508 |
| FADD | 0.512 | 0.727 | −0.106 | −0.014 | 0.264 | 0.777 | 0.038 | 0.179 | −0.117 | −0.443 |
| FAHD1 | −0.822 | −1.104 | −0.171 | 0.488 | −0.27 | −0.539 | 0.151 | −0.094 | −0.404 | −0.111 |
| FAHD2A | 0.275 | −0.032 | 0.587 | −0.054 | 0.362 | −0.247 | −0.15 | 0.026 | −0.079 | 0.007 |
| FAM38A | 1.8625 | 2.2415 | −1.0815 | 0.3835 | 2.9195 | −0.7505 | 1.8425 | 1.4365 | −0.2015 | −0.5085 |
| FAM3A | −0.3795 | −0.9885 | 0.3565 | −0.5095 | −0.5695 | 0.2355 | −0.1075 | 0.3785 | 0.3235 | 0.2495 |
| FAM3B | 0.374 | 0.338 | 0.628 | 0.858 | −0.256 | 1.38 | −0.205 | −0.141 | −0.528 | 0.684 |
| FAM3C | −1.353 | −1.845 | −0.113 | −0.685 | −0.481 | −0.228 | −0.283 | −0.9 | −0.406 | −0.257 |
| FAM46C | −0.382 | 0.761 | −0.49 | −1.094 | −1.093 | 1.078 | −0.952 | −0.442 | −1.075 | −1.538 |
| FAM49B | −0.053 | −0.633 | −1.243 | −1.592 | −1.347 | −0.764 | −1.717 | −1.6 | −1.193 | −1.126 |
| FAM54A | 0.258 | 0.516 | 0.17 | −0.526 | −0.775 | 0.379 | −0.668 | −0.993 | −0.664 | −0.416 |
| FANCA | 0.8255 | 0.8865 | 0.2725 | −0.4795 | −0.2005 | 0.4685 | −0.2565 | 0.0835 | 0.2045 | 0.1865 |
| FANCE | −1.4325 | −1.0085 | 0.7725 | 0.1105 | 0.1905 | 0.8525 | 0.6515 | 0.8225 | −0.0235 | −0.1975 |
| FBL | −0.218 | 0.134 | 0.24 | −0.412 | −0.199 | 0.004 | −0.663 | −0.426 | 0.073 | −0.21 |
| FBP1 | 0.091 | −0.685 | −0.194 | −0.064 | −0.037 | −0.018 | −0.243 | −0.638 | −0.156 | −0.129 |
| FBS1 | −0.3585 | 0.2885 | 0.2995 | 0.6725 | 0.1035 | 0.0005 | 0.1325 | 0.4045 | 0.1135 | −0.1145 |
| FBXL20 | −0.219 | −0.239 | 0.344 | −0.483 | −0.178 | −0.953 | −0.499 | −0.082 | −0.267 | 0.333 |
| FBXO25 | −0.024 | 0.141 | 0.103 | 0.018 | −0.343 | 0.579 | −0.664 | 0.669 | 0.575 | 0.403 |
| FDXR | 0.237 | 0.184 | 0.164 | 0.999 | −0.361 | −0.206 | −0.11 | 0.109 | −0.308 | −0.071 |
| FEM1B | −0.642 | −0.419 | 2.977 | 0.735 | 0.432 | 0.549 | 1.111 | 0.84 | −0.927 | −0.054 |
| FGF12 | 0.1825 | −0.0285 | −0.1655 | −0.0565 | −0.0445 | 0.5775 | −0.3735 | −0.4045 | −0.1765 | 0.4575 |
| FGF13 | 0.33 | 0.544 | 0.606 | −0.028 | −0.851 | 1.14 | −0.076 | −0.404 | −0.055 | 0.723 |
| FHOD1 | −0.1665 | −0.3555 | −0.2655 | 0.7935 | 0.9055 | 0.0625 | 0.4905 | 0.2825 | 1.8805 | 0.5175 |
| FKBP11 | −0.2425 | −0.8785 | −0.0095 | −0.1585 | −0.7795 | −0.5675 | 0.8355 | 1.8885 | 0.7225 | −0.6675 |
| FLJ10094 | 1.236 | 1.124 | 0.2 | 0.719 | 1.07 | 0.447 | 0.841 | 1.619 | 0.39 | 0.911 |
| FLJ10115 | −0.2655 | 0.0515 | 0.5185 | −0.4495 | −0.5195 | −0.1175 | −0.3355 | −0.4205 | −0.0075 | −0.4945 |
| FLJ10159 | −0.106 | 0.226 | −0.279 | −0.068 | 0.088 | 0.117 | −0.129 | −0.009 | 0.187 | −0.123 |
| FLJ10204 | −0.929 | −0.798 | 0.005 | 0.062 | −0.388 | −0.412 | −0.503 | −0.503 | −0.439 | −0.485 |
| FLJ10275 | 0.7825 | 0.1675 | 0.3465 | −0.2735 | 0.6735 | 0.6885 | 0.0895 | 1.0965 | 0.6825 | 0.9125 |
| FLJ10292 | −0.2835 | −0.0525 | −3.0215 | −0.0845 | −0.8745 | −0.2555 | −0.1155 | −0.1015 | −0.1465 | −0.3655 |
| FLJ10324 | −0.5525 | −0.1955 | −0.2565 | −0.2595 | −0.7625 | −0.0895 | −0.2935 | −0.6885 | 0.3385 | −0.5865 |
| FLJ10700 | −0.182 | −0.103 | 0.151 | 0.277 | 0.211 | −0.333 | 0.114 | −0.076 | −0.01 | 0 |
| FLJ10706 | 0.212 | 0.471 | −0.414 | −0.508 | 0.411 | −0.737 | −0.784 | −1.259 | −0.164 | −0.833 |
| FLJ10671 | −0.246 | 0.424 | 0.145 | −0.602 | 1.051 | −0.399 | 0.072 | 0.651 | 0.287 | 0.263 |
| FLJ10901 | −0.2655 | −1.1135 | −0.0885 | 0.7735 | −0.0825 | 0.4595 | 1.0225 | 0.4935 | 0.3345 | −0.6035 |
| FLJ10918 | 0.329 | 0.105 | 0.738 | 0.257 | −0.206 | 0.049 | −0.136 | −0.699 | −0.819 | −0.497 |
| FLJ10980 | 0.1725 | −0.9655 | 0.1955 | −0.3725 | 0.3385 | 0.0405 | 0.2925 | 0.4015 | 0.0915 | −0.2345 |
| FLJ11017 | −0.4575 | −0.3585 | 0.0445 | 0.1435 | 0.0085 | 0.3385 | −0.2275 | 0.1855 | 0.0175 | −0.1715 |
| FLJ11088 | 0.2965 | −0.5235 | −0.4655 | 0.5245 | −0.1675 | 0.3625 | 0.2075 | 0.0525 | −0.2605 | −0.5025 |
| FLJ11151 | −0.6415 | −0.3335 | 0.3095 | −0.0775 | 0.3385 | 0.1695 | 0.1035 | −0.0255 | 0.4945 | 0.1185 |
| FLJ11267 | −0.4055 | −0.6025 | −0.3335 | 0.8535 | 0.0125 | −0.2455 | 0.1045 | 0.0325 | −0.0115 | 0.0865 |
| FLJ11280 | 0.7735 | 0.0135 | 0.0095 | −0.7575 | −0.1475 | 0.0605 | −0.2765 | −0.1095 | −0.2305 | −0.2005 |
| FLJ11508 | 0.5045 | −0.0925 | −0.0585 | 2.0435 | 0.4915 | 0.5785 | −0.0185 | 0.4275 | 0.0035 | 0.3675 |
| FLJ12270 | −0.0785 | −0.2905 | 1.1095 | −2.2685 | −0.2045 | −1.2325 | −1.3585 | −0.3605 | −0.8135 | −0.1655 |
| FLJ12844 | 0.0075 | −0.4625 | 0.3045 | −0.6955 | −0.2485 | −0.1216 | −0.3445 | 0.4475 | 0.6235 | 0.2005 |
| FLJ12650 | −0.0145 | 0.3085 | −0.5445 | −0.5465 | −0.1955 | 0.0005 | −0.3985 | −0.8005 | −0.7915 | −0.9285 |
| FLJ12684 | 0.0585 | 1.3745 | 0.1275 | −0.1985 | 0.0425 | −0.4555 | −0.4865 | −0.0065 | −0.2935 | 0.0925 |
| FLJ12735 | 0.281 | 0.521 | 0.465 | 0.67 | 0.621 | −0.553 | 0.731 | 1.076 | −0.348 | 0.137 |
| FLJ12750 | 0.051 | −0.194 | 1.349 | 0.838 | 0.164 | 0.701 | 1.35 | 1.279 | 0.362 | 0.441 |
| FLJ12895 | −0.4695 | 0.0745 | 0.3055 | −0.7735 | −0.4895 | −0.1175 | −0.5785 | −0.0705 | −0.3555 | 0.1345 |
| FLJ13710 | −0.0415 | 0.3955 | 0.3155 | −0.9945 | −0.7875 | 0.0835 | −1.0115 | −0.8965 | −0.5375 | −1.0695 |
| FLJ13855 | 0.333 | −0.055 | −0.259 | −0.359 | −0.741 | −0.377 | 0.047 | −0.475 | 0.032 | 0 |
| FLJ13912 | −0.539 | −0.076 | −0.23 | −1.211 | −0.839 | −0.616 | −1.262 | −0.436 | −0.653 | −0.695 |
| FLJ14124 | 0.121 | −0.173 | −0.199 | −0.35 | −0.844 | −0.510 | −0.458 | −0.496 | −0.267 | −0.523 |
| FLJ14627 | −8.0795 | −0.3525 | −0.0105 | 0.1715 | 0.0035 | −0.0845 | 0.1855 | −0.0505 | −0.0775 | 0.2555 |
| FLJ14666 | −0.584 | −1.214 | −0.462 | −0.083 | −0.169 | 0.535 | 0.28 | 0.12 | −0.015 | −0.276 |
| FLJ14904 | −0.684 | −1.027 | 2.054 | 0.428 | 1.544 | 0.275 | 0.59 | 0.182 | −0.499 | −0.795 |
| FLJ20035 | −0.741 | −0.889 | −0.205 | −0.62 | −0.411 | −0.545 | −0.427 | 0.038 | −0.187 | −0.521 |
| FLJ20152 | 0.182 | 0.396 | 0.091 | −0.441 | −0.22 | −0.168 | −0.325 | −0.267 | −0.07 | −0.197 |
| FLJ20171 | −0.441 | −0.331 | 1.018 | 1.27 | −0.082 | −0.06 | 1.278 | 0.992 | 0.77 | −0.343 |
| FLJ20244 | 0.24 | 0.282 | 0.056 | 0.151 | −0.13 | −0.330 | 0.08 | 0.487 | 0.179 | −0.975 |
| FLJ20273 | 0.127 | 0.005 | −0.311 | 0.221 | 0.566 | 0.778 | 0.329 | −0.168 | −0.165 | 1.074 |
| FLJ20315 | −0.064 | −0.435 | −0.605 | −8.839 | −0.43 | 0.072 | −0.556 | 0.001 | −0.015 | −0.247 |
| FLJ20323 | −0.6935 | −1.0785 | 0.4125 | −0.3145 | −0.4525 | −0.0305 | −0.0525 | −1.1085 | −0.4885 | −0.4985 |
| FLJ20518 | 0.027 | −0.576 | 0.217 | 0.05 | 0.044 | 0.382 | 0.889 | −0.227 | −0.018 | −0.45 |
| FLJ20530 | 0.027 | −0.701 | −0.486 | −0.728 | −1.124 | −0.929 | −0.701 | −0.555 | −0.844 | −0.24 |
| FLJ20696 | −0.1515 | −0.4215 | 0.2305 | −0.3765 | −0.6885 | 0.2945 | −0.5735 | −0.1885 | −0.3305 | −0.6135 |
| FLJ20718 | −0.269 | −0.135 | 0.621 | 0.096 | 0.391 | −0.054 | 0.342 | −0.241 | −0.374 | −8.771 |
| FLJ20772 | 0.1225 | 0.2545 | −0.1955 | 0.4185 | 0.1325 | 0.0925 | −0.1235 | 0.0845 | 0.1285 | 0.1385 |
| FLJ20989 | −0.0765 | −0.3635 | 0.6255 | 1.2065 | 1.8425 | 1.3145 | 1.7795 | 1.3415 | 0.6315 | 0.8995 |
| FLJ21019 | −0.2035 | −0.3205 | −0.0845 | 1.1855 | 0.1965 | −0.7815 | 1.2685 | 1.1325 | 1.0055 | 0.3495 |
| FLJ21062 | 0.4165 | 0.6785 | 0.2605 | 0.3155 | 0.0715 | 0.3075 | 1.0895 | 0.4835 | 0.9165 | 0.5695 |
| FLJ21159 | −0.0965 | 0.2505 | 0.4225 | 1.0805 | 0.0695 | 0.2245 | 0.4885 | 0.7935 | 0.8505 | 0.0176 |
| FLJ21616 | 0.231 | 0.354 | 0.04 | 0.423 | 1.228 | 1.058 | 1.436 | 0.313 | 0.302 | −0.538 |
| FLJ21827 | 0.0355 | −0.0335 | 0.2995 | 0.2195 | 0.1755 | 1.2405 | 0.6195 | 0.1435 | 0.0385 | −0.1835 |
| FLJ21963 | −0.316 | 0.064 | 0.131 | −0.688 | −0.458 | −0.051 | −0.42 | 0.248 | −0.132 | 0.337 |
| FLJ22104 | −0.4285 | −0.4345 | 0.4755 | −0.5325 | −0.4785 | 0.7015 | −0.4495 | −0.4975 | 0.0265 | 0.2045 |
| FLJ22222 | −0.004 | −0.136 | −0.293 | 0.188 | −0.345 | 0.094 | 0.349 | 0.106 | 0.058 | −0.008 |
| FLJ22573 | −0.4995 | −0.7735 | 1.1975 | 2.0785 | 1.2165 | 1.0895 | 0.7895 | 1.1665 | −0.1745 | 1.0615 |
| FLJ22794 | 0.07 | 0.423 | 0.158 | −0.355 | −0.312 | −0.395 | −0.148 | 0.192 | 0.261 | −0.514 |
| FLJ23188 | −0.3735 | −0.3605 | −0.8805 | −0.9745 | −1.1795 | −0.3625 | −1.6345 | −0.3045 | −0.3705 | −0.3505 |
| FLJ23441 | −0.712 | −0.668 | 0.168 | 0.016 | −1.253 | −0.242 | 0.304 | −0.185 | 0.088 | −0.018 |
| FLJ25471 | −0.23 | −0.123 | 0.162 | −0.377 | −0.138 | −0.024 | 0.354 | 0.181 | −0.343 | −0.548 |
| FLJ31204 | 0.9835 | 0.8385 | −1.3825 | −0.8845 | −0.6145 | 0.3335 | −0.9375 | −1.1615 | −0.1126 | 0.3425 |
| FLJ31795 | 0.0895 | 0.7265 | 0.3725 | −0.2185 | −0.8135 | 0.0345 | −0.0765 | −0.8995 | −0.3665 | 0.4955 |
| FLJ32942 | −0.4805 | −1.4595 | 1.4765 | 1.4845 | 1.5945 | 0.8845 | 1.4785 | 2.0335 | 0.4645 | 8.1315 |
| FLJ37970 | 0.424 | −0.028 | 0.004 | −1.243 | −0.180 | 0.568 | 0.275 | 0.251 | 0.182 | −1.133 |
| FLJ39370 | −0.2705 | −0.0775 | −0.0955 | −1.1185 | −0.1745 | 0.0075 | −0.9685 | −0.0805 | 0.2305 | −0.1505 |
| FLJ90588 | 1.7816 | 1.9405 | −0.0685 | −0.2795 | −0.5735 | −0.1725 | −0.4585 | −0.1365 | 0.1425 | 0.4735 |
| FN3KRP | −2.216 | −2.178 | −0.236 | 1.096 | 0.517 | −0.708 | 0.946 | 0.438 | −0.226 | −0.634 |
| FOLR1 | 1.5545 | 3.1065 | 0.7775 | 0.1505 | 0.6805 | 1.4005 | −0.2395 | −0.2395 | 0.4735 | 1.3305 |
| FOXA1 | −0.2595 | 0.2125 | 0.2305 | 1.0355 | 0.6425 | 0.4005 | 0.1985 | 0.4895 | 0.2425 | 0.8035 |
| FOXC1 | −0.845 | −0.408 | 0.199 | −0.802 | −0.78 | −0.361 | −1.132 | −0.386 | 0.111 | 0.098 |
| FOXP1 | 0.7585 | 0.8585 | 0.2165 | 0.1425 | −0.3065 | −0.3485 | −0.1025 | 1.8805 | 0.8895 | 0.5275 |
| FREQ | −0.4095 | −0.5495 | 0.3965 | 0.2385 | −0.1045 | 0.0085 | 0.0225 | 0.1215 | −0.2005 | −0.2165 |
| FTO | −0.5125 | −0.7145 | 0.0355 | 0.9055 | 0.9645 | 0.2415 | 0.8515 | 0.2885 | 0.5735 | 0.2805 |
| FUBP1 | −0.7395 | −1.0316 | 0.5815 | 0.7595 | 0.8105 | −0.1425 | 1.3425 | 1.2815 | 0.6535 | 0.3005 |
| FUCA1 | 0.413 | 0.349 | 0.773 | 0.314 | −0.031 | −0.42 | 0.392 | 1.078 | 0.209 | 0.237 |
| FUT8 | −0.2095 | −0.9135 | 0.2225 | 0.9255 | 0.7455 | 0.7825 | 0.2555 | 0.1085 | 0.2315 | 0.0815 |
| FXYD6 | 1.229 | 1.745 | 0.391 | 0.433 | −0.558 | 0.235 | 0.067 | 0.37 | −0.137 | 0.94 |
| FYCO1 | 1.468 | 1.387 | 0.337 | 0.548 | 0.104 | 0.263 | 0.895 | 1.91 | 1.444 | 0.882 |
| FZD10 | 0.7125 | 0.9195 | −0.0975 | −0.0875 | −0.4485 | −0.0055 | −0.5275 | −0.4795 | 0.8895 | −0.0285 |
| FZD7 | −1.9405 | −1.4795 | −1.9175 | −1.6655 | −1.4815 | −0.2475 | −2.3065 | −1.1385 | −1.5625 | −0.7645 |
| FZD9 | −0.8525 | −0.9125 | −0.8735 | −0.3605 | −0.5965 | −0.6605 | −1.4365 | −0.7225 | −1.0745 | −0.3445 |
| G1P2 | 0.2755 | 0.1895 | 0.3155 | 0.2855 | −0.0205 | −0.0105 | 0.0065 | 0.3775 | 0.0825 | −0.0975 |
| G1P3 | 0.104 | 0.12 | 0.237 | −0.197 | 0.145 | 0.462 | 8.126 | 0.317 | 0.046 | 0.343 |
| GA17 | −1.0895 | 0.5375 | 0.3385 | −1.6475 | −0.8235 | −1.5185 | −0.6215 | −0.7305 | −0.7475 | −1.4435 |
| GABARAP | −0.7115 | −0.5085 | −0.6535 | −0.0575 | −0.7625 | −0.5035 | −1.1135 | −0.5065 | −0.0795 | −0.2855 |
| GAJ | 0.1165 | 1.1885 | −0.2755 | −1.1945 | −1.7245 | −0.5485 | −0.4275 | −0.0785 | −0.2385 | −0.1425 |
| GALE | −0.584 | −0.176 | −0.506 | 0.82 | −0.173 | −0.205 | −0.537 | 0.027 | 0.004 | −0.181 |
| GALNT14 | −0.8145 | −2.2335 | 1.3955 | 1.1475 | 0.3115 | −0.5275 | 1.2865 | 1.4675 | 0.7715 | 0.0375 |
| GALNT2 | 0.5375 | 0.5585 | 0.2415 | 0.2415 | 0.3585 | 0.2505 | 0.3025 | 0.6635 | 0.2405 | −0.1755 |
| GALNT7 | 0.278 | −0.118 | −0.881 | −0.5 | 0.512 | −0.532 | −0.853 | −0.391 | 0.06 | −0.536 |
| GAMT | −0.948 | −0.211 | −0.299 | −0.448 | −0.263 | −0.227 | −0.556 | −0.642 | −0.695 | 0.05 |
| GAPD | −0.498 | −0.168 | −0.284 | −0.428 | −0.169 | −0.609 | −0.125 | −0.457 | −8.008 | −0.3 |
| GARS | −1.385 | −1.035 | 0.224 | 0.724 | 0.914 | −0.568 | 0.487 | −0.164 | 0.777 | −0.655 |
| GART | −0.8175 | −1.7475 | 0.9985 | −0.6215 | 0.1375 | 1.1795 | 0.3815 | 1.1635 | 1.5645 | −0.8915 |
| GATA3 | −1.1965 | −0.5575 | 0.1835 | −1.0505 | −0.2215 | −0.3495 | −0.6255 | −0.4015 | −0.0565 | −0.3335 |
| GATM | 0.008 | 0.223 | 0.865 | −0.622 | −0.752 | −0.23 | 0.026 | 0.345 | 0.184 | −0.47 |
| GBA | −0.023 | −0.021 | 0.5 | −0.722 | −0.464 | 0.227 | −0.289 | 0.028 | 0.141 | 0.021 |
| GDAP1 | −0.118 | −0.212 | −0.116 | −0.469 | −0.855 | 0.115 | −0.187 | −0.086 | −0.048 | 0.129 |
| GDI2 | −0.1725 | −0.0505 | 0.0765 | 0.0695 | 0.0255 | 0.4175 | 0.3455 | 0.0165 | 0.1065 | −0.8255 |
| GGA1 | −0.2505 | −0.4055 | −0.0125 | 0.7385 | 1.3835 | 0.5795 | 0.8435 | 0.0075 | 0.3855 | −0.4105 |
| GKAP1 | 0.2135 | 0.3075 | 0.1155 | 0.1815 | −0.0265 | 0.0745 | −0.4855 | 0.0895 | 0.2225 | 0.5295 |
| GLCCI1 | 1.3265 | 1.6845 | 0.7455 | 0.3416 | 0.7525 | 1.0915 | 0.2065 | 0.5775 | 0.8816 | 0.8295 |
| GLG1 | −0.129 | 0.181 | 0.152 | −0.813 | −0.994 | 0.948 | −0.889 | −0.3 | 0.223 | −0.014 |
| GLTSCR2 | −0.725 | −1.059 | 0.943 | 0.588 | −0.167 | 0.473 | 0.356 | 0.821 | 0.169 | 1.228 |
| GNB4 | −0.222 | −0.102 | −0.158 | 0.485 | 0.217 | −0.048 | 0.532 | −0.078 | −0.069 | −0.215 |
| GOLPH2 | 0.4345 | −0.0565 | −0.4305 | −0.3715 | −0.6205 | −0.1815 | −0.2625 | −0.4445 | −0.1935 | −0.0555 |
| GPATC1 | 0.7145 | 0.5605 | 0.3575 | −0.1955 | −1.0225 | −0.3835 | 0.0465 | 0.3745 | 0.4595 | −0.2025 |
| GPC2 | −0.8975 | −1.6645 | 1.1875 | 1.1535 | 0.1365 | 0.5055 | 1.0335 | 0.8355 | 0.7635 | −0.0515 |
| GPR125 | −0.44 | 0.264 | 0.26 | −0.163 | −1.094 | −0.397 | −1.487 | −0.16 | −0.47 | −0.596 |
| GPR160 | −0.1645 | −0.1105 | 0.1345 | 0.5485 | −0.5285 | 0.8115 | −0.6215 | 0.4745 | −0.4265 | 0.1225 |
| GPR56 | 0.8145 | 0.6305 | 0.4965 | −0.1985 | −0.1075 | 0.1855 | −0.3325 | 0.1985 | −0.3925 | 0.4835 |
| GPRC5C | −0.6885 | 0.9525 | −0.5185 | −0.7185 | −0.8145 | −0.6835 | −0.9355 | −0.3415 | −0.6585 | −1.0675 |
| GPSM1 | −0.198 | 1.464 | −1.22 | 0.554 | 0.31 | 0.42 | −0.515 | 0.746 | 1.615 | 0.311 |
| GPSM2 | −0.095 | 0.391 | −0.248 | −0.043 | 0.713 | 0.547 | −1.183 | −0.8 | −0.69 | −1.319 |
| GPT | 0.3085 | 0.3835 | 0.8495 | −0.1865 | 0.0915 | −0.0335 | −0.3005 | −0.1095 | −0.0015 | 1.2865 |
| GPT2 | −0.089 | −0.39 | 0.587 | 0.078 | −0.427 | −0.311 | −1.084 | 0.507 | −0.367 | 0.452 |
| GPX7 | −0.108 | −0.025 | −0.261 | −0.248 | −0.292 | −0.175 | −0.019 | −0.349 | 0.154 | 0.134 |
| GRB14 | 0.3245 | 0.1165 | 0.1345 | 1.4625 | 2.5735 | 0.9295 | 0.8865 | 1.1145 | −0.0155 | 0.4125 |
| GRB7 | 0.1235 | 0.0695 | −0.2165 | −0.4545 | −0.3965 | −0.1145 | −0.4205 | −0.4875 | −0.7305 | −0.5015 |
| GSC | −0.482 | −0.023 | −0.217 | −0.097 | −0.062 | −0.297 | −0.123 | −0.057 | 0.291 | −0.186 |
| GSDML | 0.138 | 0.085 | 0.157 | −0.179 | 0.09 | 0.336 | 0.041 | 0.213 | −0.25 | 0.299 |
| GSK3B | 0.031 | 0.223 | 0.046 | −0.123 | 0.607 | −0.432 | 0.413 | 0.436 | 0.013 | −0.091 |
| GSTA1 | 0.1645 | −0.1375 | −0.9925 | 0.6385 | 1.5075 | 0.4915 | 1.6925 | 1.3925 | −0.2755 | −0.7475 |
| GSTK1 | −0.165 | 0.403 | 1.976 | 0.118 | −0.429 | −0.216 | −0.247 | 0.41 | −0.598 | 0.558 |
| GSTM3 | −0.1975 | 0.5385 | 1.1355 | 0.4395 | 0.8065 | −0.4085 | 0.2055 | 0.3275 | −0.0076 | −1.9205 |
| GSTP1 | 0.543 | −0.79 | −0.254 | 0.274 | 0.024 | 1.022 | 0.8 | 0.596 | 1.251 | 1.108 |
| GSTT1 | 0.1475 | 0.1355 | 0.2135 | 0.0565 | −0.4565 | −0.3935 | −0.1635 | 0.4255 | 0.2005 | 0.1405 |
| GSTT2 | 0.0155 | −0.3805 | −0.3485 | 0.0895 | 0.0485 | −0.3645 | 0.1005 | 0.1625 | 0.1145 | −0.2465 |
| GTF2F2 | −0.485 | −0.578 | −0.396 | −0.431 | −0.535 | −0.225 | −0.089 | −0.389 | −0.036 | −0.295 |
| GTF2I | −0.1765 | −0.0205 | 0.0645 | 0.0735 | −0.2605 | −0.4445 | −0.2955 | −0.5455 | −0.4095 | −0.8345 |
| GTF3A | −0.362 | −0.515 | −0.144 | 0.016 | −0.053 | −0.26 | −0.331 | −0.519 | −0.175 | −0.005 |
| GTPBP3 | −0.298 | 1.552 | 0.768 | −1.04 | −0.284 | −0.009 | 0.004 | 0.188 | 0.924 | −0.199 |
| GTPBP4 | −0.721 | −1.46 | 0.038 | −0.211 | −0.232 | −0.638 | −0.474 | −0.575 | −0.456 | −0.552 |
| H1F0 | −0.2335 | −0.4375 | −0.2385 | 0.0255 | −0.4405 | −0.5215 | −0.8215 | −0.4085 | −0.0815 | −0.8525 |
| H2AFV | −1.0745 | −1.6075 | 0.0725 | −0.6425 | −0.7975 | −0.4705 | −0.5485 | −0.5765 | −0.6945 | −1.0115 |
| H2AFY | −0.5835 | 0.3565 | −0.9165 | 0.9435 | −1.2115 | 0.0895 | −0.7315 | −0.8285 | −0.6465 | −1.4815 |
| H2AFZ | −0.2615 | 0.0825 | 0.8125 | −1.1925 | −0.3605 | 0.8955 | −0.4255 | 0.0115 | −0.1885 | 0.1465 |
| H2BFS | −0.1125 | 0.5695 | 0.1035 | −0.5585 | −0.1035 | 0.0045 | 0.1015 | 0.0465 | −0.0045 | 0.0685 |
| H3F3B | −0.54 | −0.091 | 0.418 | −0.647 | 0.376 | 0.345 | −0.314 | −0.372 | −0.174 | −0.545 |
| H41 | 0.3655 | 0.4225 | −2.4375 | −0.0275 | −0.0145 | 0.3735 | 0.2275 | −0.3645 | 0.5416 | 0.3365 |
| HAGH | −0.18 | −0.311 | 0.36 | −0.185 | −0.553 | −0.152 | 0.007 | −0.117 | 0.153 | −0.283 |
| HBG1 | −1.5075 | −0.8505 | −1.0935 | −1.7495 | −1.4825 | −1.5526 | −1.0935 | −1.5255 | −0.7585 | −1.1815 |
| HBL01 | −0.314 | −1.115 | 0.171 | −0.037 | −0.115 | −0.241 | −0.079 | −0.437 | −0.918 | −0.101 |
| HCAP-G | −0.0225 | −0.4105 | −0.0615 | −0.1205 | −0.7005 | −0.7925 | −0.5055 | −0.2765 | −0.2015 | −0.1685 |
| HCP5 | 0.01 | −0.317 | 2.303 | 0.031 | 0.388 | 4.129 | 0.945 | 1.347 | 0.539 | 2.764 |
| HDAC2 | −0.352 | −0.54 | 0.312 | 0.031 | 0.383 | 0.088 | 0.238 | 0.057 | 0.461 | −0.031 |
| HDC | −0.1805 | 0.1715 | −0.0715 | 0.1245 | 0.3865 | −0.5065 | 0.2645 | 0.1215 | 0.2345 | 0.2305 |
| HEBP1 | −0.79 | −0.101 | −0.528 | 0.861 | 0.117 | −0.6 | 0.308 | 0.053 | 0.44 | −0.585 |
| HIPK2 | −0.568 | −0.019 | −0.49 | 0.025 | −0.191 | −0.399 | 0.236 | −0.359 | 0.408 | 0.015 |
| HIPK2 | −1.2935 | −0.3415 | −1.6235 | 0.1535 | −0.8595 | −0.0155 | −0.2995 | −0.7735 | −1.3855 | −1.1345 |
| HIS1 | −0.9175 | −0.1185 | −1.0825 | −0.2625 | −0.8545 | −0.2625 | −0.1815 | −0.8545 | −1.0126 | −1.2825 |
| HIST1H2A | −0.946 | −0.272 | −1.142 | −0.352 | −0.812 | −0.267 | −0.339 | −1.021 | −1.101 | −1.362 |
| HIST1H2B | −0.8465 | −0.5055 | −1.2055 | −0.5645 | −0.8835 | −0.1605 | −0.4995 | −1.0825 | −1.1116 | −1.3115 |
| HIST1H2B | −1.027 | −0.925 | −1.195 | −0.392 | −0.896 | −0.445 | −0.347 | −0.915 | −0.93 | −1.235 |
| HIST1H2B | −0.0455 | −0.0585 | 0.7485 | −0.8325 | 0.1875 | 0.3515 | −0.0055 | 0.3205 | −0.0005 | 0.3215 |
| HIST1H2B | −0.5555 | −0.2285 | 0.3465 | −0.5795 | −0.8785 | −0.2325 | −1.1865 | −0.6965 | −0.6215 | −0.4195 |
| HIST1H3C | −0.3925 | −0.8725 | −0.7525 | −1.1365 | −1.9155 | −1.0325 | −1.2745 | −0.8355 | −0.3765 | −0.8375 |
| HM13 | −1.8885 | −1.4985 | −1.3225 | −1.3015 | −1.0495 | −1.3515 | −1.1795 | −1.3505 | −0.5145 | −0.6545 |
| HMGB3 | −0.5475 | −0.7595 | 0.0585 | 0.1315 | −0.5975 | −0.3475 | −0.4605 | −0.2475 | −0.2195 | −0.0395 |
| HN1 | 0.172 | 0.249 | 0.346 | −0.549 | 0.139 | 0.61 | −0.191 | 0.775 | 0.42 | 0.32 |
| HNRPA3P | 1.3765 | 1.3225 | 1.3065 | 1.5215 | 1.6815 | 0.2465 | 1.8425 | 1.2125 | 0.5055 | 0.2625 |
| HNRPDL | 1.271 | 1.581 | 1.602 | 1.725 | 1.798 | 1.228 | 2.727 | 1.41 | 1.404 | 0.908 |
| HOXA5 | −0.9305 | −1.1385 | 0.9585 | 0.5535 | 0.9165 | −0.3445 | −1.4285 | 1.3135 | 1.4015 | 0.0445 |
| HOXA7 | 0.3475 | −0.8865 | 0.3445 | 1.7025 | −0.1045 | −0.0705 | −0.0975 | 2.9255 | 2.6685 | −0.0565 |
| HOXB2 | −1.1815 | 0.2325 | −0.0065 | 0.3775 | 0.5745 | 0.2345 | −1.0635 | 0.0455 | 0.5935 | −0.2765 |
| HOXB6 | −0.7635 | −0.4475 | 0.3545 | 2.6575 | 1.1505 | −0.6005 | −0.3805 | 0.1685 | −0.3955 | 0.7235 |
| HOXB7 | −0.0365 | 0.3635 | −0.2725 | 1.0315 | 0.2055 | −0.5925 | −1.1045 | −0.5495 | −0.4235 | −0.0925 |
| HOXC10 | −0.878 | −1.038 | −0.282 | 1.004 | 0.304 | −0.427 | 0.253 | −0.224 | −0.588 | 0.236 |
| HOXC13 | 0.3445 | 1.4865 | −1.3225 | 0.0735 | −0.0535 | 1.0925 | −0.4015 | 0.6185 | 1.5115 | 0.3635 |
| HOXC9 | −0.035 | −0.182 | 0.265 | 0.048 | 0.206 | 0.558 | 0.532 | 0.507 | 0.885 | 0.237 |
| HOXD3 | −0.5215 | 0.0085 | 0.0985 | −0.4785 | −0.4475 | −0.3185 | 0.0035 | −0.5605 | −0.3416 | −0.9855 |
| HPCL2 | 0.064 | 0.37 | −0.78 | 0.222 | −0.395 | 0.568 | 0.72 | −0.152 | 0.558 | −0.56 |
| HPS3 | −0.6015 | 0.1385 | −0.2485 | 0.0485 | −0.0595 | −0.1265 | −0.0485 | −0.2345 | −0.1825 | −0.3365 |
| HRASLS | −0.5965 | −0.4025 | 0.9145 | −0.3315 | 0.1875 | 0.3285 | −0.3685 | −0.0385 | −0.5395 | −0.9635 |
| HRB | 1.005 | 0.848 | −0.389 | −1.281 | −1.094 | 0.264 | 0.572 | 0.795 | 0.142 | 0.51 |
| HRSP12 | −0.375 | 0.07 | 0.354 | −0.816 | −0.247 | −0.193 | −0.72 | −0.115 | −0.346 | −0.271 |
| HSD17B2 | −0.5045 | −0.4535 | −0.2355 | 0.0845 | −0.5295 | −0.0625 | −0.3125 | −0.0785 | −0.0375 | −0.4025 |
| HSF1 | −0.2895 | −1.2055 | 1.9945 | −0.6715 | 0.1005 | 0.7925 | 0.8285 | −0.1215 | −0.4705 | −0.1945 |
| HSPA14 | −0.847 | −1.624 | −0.758 | −0.099 | −1.206 | −0.693 | −0.295 | 0.383 | −0.715 | −0.388 |
| HSPA2 | −0.2615 | −0.6725 | −0.0115 | 0.3175 | −0.4075 | 0.0725 | 0.0425 | −0.0825 | 0.2715 | −0.2515 |
| HSPA5BP [symbol] | 0.01 | −0.06 | −0.338 | 0.542 | 0.124 | 0.385 | −0.179 | −0.042 | 0.197 | −0.015 |
| HSPC016 | 0.015 | −0.195 | −0.036 | 0.405 | 0.139 | 0.023 | 0.136 | 0.132 | 0.067 | −0.113 |
| HSPC138 | −1.221 | −1.576 | −0.622 | −1.144 | −1.02 | −1.548 | −0.471 | −1.107 | −0.878 | −0.927 |
| HSPC148 | −1.0445 | −0.8485 | −0.1645 | −0.5185 | −0.1475 | −0.7145 | −0.0955 | −0.6565 | −0.3555 | −0.4055 |
| HSPC150 | −0.1476 | 0.0555 | 0.0455 | 0.0225 | −0.3105 | 0.4335 | −0.1905 | 0.0165 | −0.2965 | −0.1545 |
| HSPC163 | 0.538 | 1.085 | 0.249 | −0.209 | −0.244 | −0.18 | −0.24 | 0.083 | 0.171 | 0.185 |
| HSPC266 | −0.6 | −0.904 | 0.118 | −1.505 | −0.788 | −0.915 | −0.589 | −0.998 | −0.501 | −0.794 |
| HSU79274 | −1.086 | −0.264 | 0.101 | −0.192 | −0.355 | 0.027 | −0.731 | −0.198 | −0.131 | −0.048 |
| HTATIP2 | −0.674 | −0.361 | −0.539 | −0.858 | −0.355 | −0.302 | −0.322 | −0.297 | −0.352 | −0.484 |
| ICMT | 1.54 | 1.856 | 0.835 | 2.103 | 1.235 | 1.224 | 1.277 | 0.879 | 1.089 | 1.407 |
| ICT1 | −1.113 | −1.273 | 0.229 | 1.289 | −0.16 | −0.226 | −0.612 | −0.295 | 0.13 | −0.081 |
| ID4 | −0.4275 | 0.0285 | 0.1785 | 0.1615 | −0.0325 | 0.0155 | 0.3435 | 0.4025 | 0.3725 | 0.2465 |
| IDH2 | −1.905 | −1.54 | −0.909 | −0.663 | −0.882 | −0.975 | −0.241 | −0.849 | −0.474 | 0.007 |
| IDS | −0.7685 | −1.6815 | −0.4585 | −0.0385 | −0.5185 | 0.3785 | −0.4015 | −0.1305 | −0.8875 | −0.3855 |
| IFI30 | −0.3835 | −0.7885 | 0.0125 | −0.2075 | −0.4325 | 0.4845 | −0.3145 | 0.0215 | −0.4065 | −0.2635 |
| IFI44 | −0.6215 | −0.3335 | 0.2975 | −0.7045 | 0.1355 | −0.0165 | −0.2805 | −0.5635 | 0.8405 | 0.1625 |
| IFIT5 | 0.4245 | 0.2725 | 0.4885 | 1.0425 | 0.6325 | 0.2485 | 0.5835 | 0.4075 | 0.2405 | 0.1895 |
| IFITM1 | −0.1925 | 0.1155 | 0.3035 | 1.0145 | 1.5885 | 1.2805 | 1.5855 | 1.7425 | 2.2915 | 0.0115 |
| IGBP1 | −0.2225 | 0.1265 | 1.0305 | −0.7185 | −0.4545 | 0.8815 | −0.0575 | 1.2195 | 0.3145 | −0.1325 |
| IGFALS | −0.6085 | −0.1535 | 1.6935 | 1.2695 | −0.3095 | 0.2005 | 0.6605 | 0.1985 | −0.2075 | 0.8145 |
| IGPBP2 | −0.4345 | −0.5835 | 1.9415 | 1.1815 | 0.0015 | 0.2205 | 0.5975 | −0.0995 | −0.1195 | 0.6295 |
| IGHG1 | 2.8895 | 2.5705 | 2.3705 | 2.1895 | −2.5405 | 3.5375 | 1.7995 | 1.6345 | 1.3025 | 1.7275 |
| IGHG1 | −0.08 | −1.064 | 1.865 | 1.676 | −0.782 | 2.319 | 1.297 | 0.814 | 0.994 | 1.847 |
| IGJ | 0.497 | −1.135 | 1.948 | 1.892 | −2.368 | 0.692 | 0.723 | 0.541 | 0.623 | 1.312 |
| IGKC | 0.213 | −0.401 | 1.182 | 0.643 | −0.257 | 0.993 | 0.434 | 0.274 | 0.452 | 0.438 |
| IGKC | 1.375 | 0.604 | 1.851 | 1.005 | −1.102 | 0.486 | 0.663 | −0.711 | 0.137 | 1.077 |
| IGLC2 | −0.9105 | −1.3445 | 0.5575 | 0.1165 | −0.7195 | 0.0535 | −0.5216 | 0.0005 | 0.2145 | −0.2025 |
| IGLL1 | 1.1245 | 1.2335 | 0.2985 | 0.9255 | −0.1185 | 0.8505 | 1.1935 | 3.4165 | −0.7955 | 0.7885 |
| IL13RA1 | −0.369 | 0.796 | −0.28 | −0.545 | −0.532 | 0.595 | 0.143 | −0.377 | 0.208 | 0.049 |
| IL17B | 0.0176 | −0.5735 | 1.9885 | 0.9095 | 1.7115 | 1.2275 | 1.7135 | 1.6175 | 0.4615 | −0.1455 |
| IL23A | −0.0735 | 0.0145 | −0.8555 | 0.3285 | −0.2545 | −0.6085 | 0.1485 | −0.4095 | −0.0095 | −0.0075 |
| IL6ST | 0.3485 | 0.3815 | −0.3465 | −0.7285 | 0.0805 | 0.0355 | −0.1535 | −0.5705 | −0.1745 | −0.3885 |
| ILF2 | −0.396 | −0.58 | −0.198 | 0.196 | 0.127 | −0.087 | 0.613 | 0.918 | 0.548 | 0.314 |
| IMPA2 | −0.0085 | 0.7035 | 0.6025 | 1.2885 | 1.0405 | 0.7695 | 0.0085 | 0.8215 | −0.6885 | 0.3295 |
| ING1L | −0.834 | −0.61 | 1.018 | −0.527 | 1.248 | 0.176 | 1.643 | 1.02 | −0.181 | −0.404 |
| INHBB | −0.0995 | 0.1625 | 0.3325 | −0.5315 | 0.8295 | −0.3725 | 0.9715 | −0.3315 | −0.4045 | −0.0145 |
| INPP4B | 0.1725 | −0.0325 | 2.2345 | 1.0425 | 1.7115 | −0.2135 | −0.7375 | 1.8795 | 0.9125 | 1.1875 |
| INSM1 | −0.857 | −0.671 | 0.081 | −0.023 | 0.526 | −0.604 | −0.186 | 0.223 | 0.399 | −0.305 |
| IQCA | −0.078 | −0.12 | 1.102 | 2.345 | 1.66 | 1.239 | 1.935 | 0.668 | −0.186 | 0.375 |
| IQCE | 1.011 | 1.319 | 0.312 | 0.133 | −0.689 | 0.568 | 0.104 | −0.831 | −0.474 | 0.46 |
| IQGAP2 | −0.2415 | −0.5045 | 0.0865 | 0.3845 | −1.1665 | 0.2075 | 0.7825 | −0.8865 | 0.3125 | 0.4345 |
| IRF4 | 1.4385 | 0.8595 | −0.0015 | 0.4425 | 0.4965 | −0.3115 | 0.6865 | 1.9125 | 1.5335 | 0.8015 |
| IRTA2 | −0.194 | 0.115 | −0.363 | −0.825 | −1.19 | 0.456 | −0.542 | −0.908 | −1.094 | −0.232 |
| IRX4 | −0.6535 | −1.4825 | −0.1015 | −0.3985 | −0.8245 | −0.5355 | −0.8995 | −0.2795 | −0.7245 | −0.4376 |
| ISG20 | 0.608 | 1.429 | 0.39 | −0.32 | −0.534 | 0.327 | 0.013 | 1.156 | 0.231 | 0.127 |
| ISGF3 [symbol] | 0.222 | −0.771 | 0.777 | 2.477 | 0.001 | 0.438 | 2.039 | 1.52 | 1.302 | 0.117 |
| ITGB4 | −0.241 | 0.292 | 0.033 | 0.182 | −0.38 | 0.228 | 0.192 | −0.324 | −0.274 | −0.281 |
| ITPR1 | −0.966 | −1.3 | −0.309 | −0.455 | 0.235 | 0.006 | 0.717 | 0.619 | 1.155 | 0.227 |
| JARID1A | −0.118 | 0.247 | −0.043 | 0.268 | −0.433 | −0.034 | 0.001 | −0.305 | 0.058 | 0.334 |
| KARCA1 | 0.221 | 0.381 | −0.465 | −0.434 | −0.369 | 1.957 | −0.566 | −0.165 | −0.118 | 0.12 |
| KATNA1 | −0.3385 | −0.1615 | 0.3776 | −0.2085 | −0.1965 | 0.0725 | 0.4955 | 0.0745 | 0.9085 | −0.0155 |
| KCNC2 | −0.189 | 0.046 | 0.68 | 0.874 | 0.432 | −0.206 | 0.292 | −0.032 | 0.471 | 0.597 |
| KCND2 | 1.718 | 1.8 | −0.163 | −0.848 | −1.471 | 0.003 | −0.748 | 0.06 | 0.473 | 0.059 |
| KCNK4 | −0.458 | −1.032 | −0.123 | −0.51 | −1.374 | 1.181 | 0.193 | −0.693 | 1.066 | −0.5 |
| KCNN4 | −0.2895 | 0.3745 | −0.0925 | 1.1995 | 0.1755 | 0.0585 | −0.1595 | 0.5175 | 0.5785 | −0.3145 |
| KCNS3 | −0.5525 | −0.7755 | 0.0355 | 0.1765 | 0.4315 | −0.2025 | 1.0775 | 1.1775 | 0.9235 | 0.0695 |
| KCTD15 | −1.756 | −1.593 | −0.606 | −0.241 | 0.352 | −0.054 | −0.904 | −0.422 | −0.217 | −0.366 |
| KCTD3 | 0.751 | 0.572 | 0.351 | −0.397 | −0.49 | 0.069 | −0.152 | 0.13 | 0.213 | 0.18 |
| KDELR2 | −1.348 | −2.194 | −0.91 | −1.252 | −0.719 | −1.735 | −0.572 | −0.702 | −0.468 | −1.742 |
| KIAA0020 | −0.355 | 0.1 | −0.456 | −0.524 | −0.553 | −0.184 | 0.451 | −0.666 | −0.573 | −0.151 |
| KIAA0101 | −0.9895 | −0.2585 | −0.6775 | 0.6965 | −0.0475 | 0.2545 | −0.2745 | −0.4485 | −0.9535 | −0.6705 |
| KIAA0153 | −0.5105 | −0.7825 | 0.3515 | 0.1785 | −0.4285 | −0.7425 | 0.0335 | −0.3175 | −0.1495 | −0.2495 |
| KIAA0182 | −0.1235 | −0.1775 | −0.5315 | −1.2095 | −0.8105 | 0.8615 | −0.0295 | −0.4655 | −0.4635 | −0.4645 |
| KIAA0196 | −0.7285 | −1.0275 | −0.1345 | 0.3825 | 0.0335 | 0.6185 | 0.3135 | −0.4815 | −0.2495 | −0.3495 |
| KIAA0241 | −0.128 | −0.28 | 0.098 | −0.441 | −0.25 | 0.316 | 0.579 | 0.664 | 0.433 | −0.084 |
| KIAA0251 | −0.8176 | 0.1555 | −0.2965 | 0.0905 | −0.0685 | 0.0835 | −0.5175 | −0.1505 | 0.0675 | 0.1755 |
| KIAA0274 | 0.141 | 0.823 | 0.893 | 0.407 | 0.997 | 0.749 | 0.479 | 0.184 | 0.258 | 0.181 |
| KIAA0515 | −0.0645 | 0.1015 | −0.4495 | −0.1165 | −0.9235 | 0.6835 | 0.3315 | −0.2875 | 0.0685 | −0.4055 |
| KIAA0582 | −0.5715 | −1.2385 | −0.2195 | 0.6455 | 0.6315 | −0.6235 | 0.1045 | 1.0545 | −0.9585 | −0.9335 |
| KIAA0650 | 0.1905 | 0.3885 | 1.2315 | −0.0825 | −0.3985 | 0.1815 | −0.5835 | 0.0095 | 0.2755 | 0.4005 |
| KIAA0703 | −0.15 | −0.671 | −0.278 | −0.799 | −0.371 | −1.165 | −0.156 | 0.22 | −0.83 | −0.215 |
| KIAA0830 | 0.0185 | −0.1275 | −1.1255 | 1.3485 | 0.5915 | −0.7925 | 0.4595 | 1.0016 | 0.3245 | −0.2325 |
| KIAA0980 | −0.5825 | −1.2315 | −0.6155 | −0.0015 | −0.1225 | 0.4525 | 0.0015 | −0.1705 | 0.4985 | 9.3965 |
| KIAA1324 | 0.557 | 1.544 | −0.102 | 0.116 | 0.555 | 0.142 | 0.658 | 1.133 | 0.038 | 0.209 |
| KIAA1363 | −0.288 | 0.358 | 0.762 | 1.046 | 0.567 | 1.545 | 1.167 | 1.004 | 0.945 | 0.787 |
| KIAA1683 | −0.0975 | 0.7295 | −1.2455 | −0.8155 | −0.9555 | 0.7625 | 0.1415 | −1.2755 | −0.8315 | −0.5505 |
| KIF12B | −0.0895 | 0.1645 | 0.1365 | −0.2425 | −0.4945 | 0.0825 | −0.1045 | −0.0575 | −0.3615 | −0.6545 |
| KIF24 | 0.1555 | 0.0385 | −0.2645 | −0.0145 | −0.1395 | −0.2295 | −0.6855 | −0.0565 | 0.3995 | 0.2305 |
| KIN | 3.0685 | 2.8285 | 2.0075 | 0.7005 | −0.0915 | 0.7765 | 1.4065 | 1.6495 | 1.2765 | 1.0595 |
| KIRREL3 | 0.7715 | 2.5275 | 0.6895 | 0.3745 | −0.3005 | 0.4155 | 0.4915 | 2.1585 | 1.0045 | 0.7905 |
| KIT | 1.9385 | 3.3845 | 0.4105 | −0.3495 | −0.8475 | 0.6045 | 0.0665 | 2.7105 | 0.5635 | 0.8295 |
| KLK5 | 0.638 | 0.953 | 0.712 | 0.821 | −0.253 | −0.503 | 1.896 | 3.389 | 0.483 | 0.657 |
| KLK6 | 1.166 | 2.392 | 1.683 | 0.399 | 1.954 | −0.099 | 0.588 | 2.499 | 0.626 | 0.742 |
| KLK7 | −1.518 | −1.231 | −1.291 | −1.465 | −1.173 | −0.953 | −1.496 | −0.936 | −0.927 | −1.49 |
| KLK8 | −0.8825 | −0.4755 | −0.9975 | −1.1355 | −0.7505 | −0.9095 | −0.4975 | −0.8465 | −0.7045 | −0.7005 |
| KNTC2 | 0.373 | −0.177 | −0.782 | −0.907 | −1.004 | 1.584 | −0.42 | −1.166 | −0.279 | −0.305 |
| KPNA2 | −0.1275 | −0.2815 | 0.2455 | −0.7895 | 0.2885 | −0.1295 | −0.1335 | 0.0075 | 0.1875 | −0.0195 |
| KREMEN2 | 3.6315 | 2.4855 | 1.8515 | 0.9375 | 3.4995 | 0.3135 | 0.1185 | 2.6015 | −0.0395 | 0.8715 |
| KRT10 | 3.5855 | 3.4435 | 2.4255 | 0.9135 | 3.2035 | 0.2015 | −0.1855 | 2.0885 | −0.3955 | 1.3815 |
| KRT13 | 1.159 | 3.354 | 0.743 | 0.007 | −0.754 | −0.266 | 0.818 | 1.884 | 2.037 | 0.966 |
| KRT15 | −0.827 | 0.041 | −0.254 | −0.749 | −0.136 | 0.059 | −0.579 | 0.378 | −0.216 | −0.031 |
| KRT17 | −0.1365 | 2.7235 | −1.2235 | 0.9015 | 1.0635 | 0.5595 | −0.4875 | 0.8515 | 0.3035 | 1.4265 |
| KRT18 | −0.148 | −0.743 | 0.957 | 0.453 | −1.418 | −1.22 | −0.467 | 2.31 | 1.842 | 1.152 |
| KRT23 | −0.2305 | −0.4935 | −0.0655 | −0.6685 | 0.2025 | 0.1605 | −0.1225 | −0.1565 | −0.4535 | −0.1475 |
| KRT6B | 0.199 | −0.385 | −0.258 | −0.601 | −0.044 | −0.254 | −0.858 | −0.033 | 0.262 | 0.135 |
| KRTAP3-3 | 1.2305 | 0.7185 | 0.3155 | 0.2265 | −0.2275 | −0.3395 | 1.0625 | 2.4475 | 0.4495 | −0.0725 |
| KRTHA7 | 1.6185 | 2.1905 | 0.3615 | 0.0645 | 0.2145 | 0.0245 | 0.9345 | 2.5945 | 1.9315 | 0.4345 |
| LAMA3 | −0.749 | −0.758 | −0.853 | −0.065 | 0.95 | −0.375 | 0.699 | −0.558 | 0.185 | −0.32 |
| LAMB3 | −0.442 | −0.694 | 1.295 | 0.343 | −0.268 | 0.747 | 0.333 | −0.377 | 0.124 | 0.265 |
| LASS2 | 0.0765 | −0.2605 | 0.0385 | −0.0925 | −0.2465 | 0.3755 | 0.1795 | −0.5965 | −0.1695 | 0.4265 |
| LAX | −0.8455 | 1.2955 | −1.9295 | 0.5505 | 0.5155 | 0.7715 | −1.6185 | 1.4005 | 1.7455 | −0.4265 |
| LBR | 1.1305 | 0.3245 | −0.0735 | −0.0965 | 0.6085 | 1.0385 | 0.3065 | 0.6315 | 0.7205 | −0.1265 |
| LCE1B | −0.1135 | −0.4265 | 1.1305 | −0.1735 | 0.1075 | −0.0865 | 0.0015 | 0.5685 | 0.7135 | 0.1745 |
| LDHB | 0.756 | 0.706 | 0.479 | 0.456 | 0.288 | 0.389 | 0.503 | 0.177 | 0.134 | −0.045 |
| LDOC1L | −0.102 | −0.018 | 0.98 | 0.96 | 1.792 | 0.773 | −0.098 | 0.208 | −1.109 | −0.329 |
| LETMD1 | −0.2785 | −0.4715 | 1.3235 | 0.2575 | −0.5045 | 0.0345 | 0.8785 | −0.8715 | −0.9525 | 0.5495 |
| LFNG | 0.395 | 0.516 | −0.246 | 0.322 | 0.831 | −0.19 | 0.59 | 0.109 | 0.522 | −0.094 |
| LGALS2 | −0.059 | −0.183 | 0.072 | 1.254 | 0.45 | −0.081 | 0.7 | 0.308 | −0.046 | −0.479 |
| LGTN | −0.583 | −0.552 | −0.336 | −0.014 | 1.549 | 0.765 | 0.252 | −0.739 | 0.961 | −0.252 |
| LNX | −0.2165 | −0.2975 | 0.6425 | 0.1985 | 0.3925 | 0.0535 | 0.4885 | 0.7365 | 0.5145 | −0.5095 |
| LOC11247 | −0.169 | −0.678 | 0.144 | 0.357 | −0.438 | −0.39 | 0.183 | −0.092 | 0.28 | 0.339 |
| LOC11492 | −0.364 | −1.252 | −0.636 | −0.425 | 0.183 | −0.25 | 0.009 | 0.149 | 0.578 | −0.413 |
| LOC11497 | −0.0395 | −0.8455 | 0.4845 | 0.3705 | 0.1275 | 0.5605 | 0.2925 | 1.8255 | −0.4835 | 0.0085 |
| LOC11621 | −0.4035 | −0.7145 | 2.1905 | 1.3025 | 0.4785 | 0.9275 | 0.2875 | 1.4615 | −0.8435 | 0.9525 |
| LOC11644 | −0.9675 | −1.2945 | 1.3515 | 0.6885 | 0.5085 | 0.2095 | 0.1005 | 0.2635 | −0.5385 | −0.5245 |
| LOC12022 | −0.1655 | −0.0405 | 0.3395 | −0.1895 | 0.0895 | −0.2405 | −0.2925 | −0.3055 | 0.1725 | −0.1945 |
| LOC12422 | 0.1835 | −0.2845 | −0.3705 | −0.1885 | 0.8225 | −0.4705 | −0.1325 | 0.2775 | 0.0665 | −0.0005 |
| LOC14654 | −1.174 | −1.582 | 1.269 | 0.844 | −0.255 | 0.155 | 0.456 | 0.731 | 0.105 | −0.12 |
| LOC15506 | 0.78 | 1.059 | −0.221 | −0.46 | −0.153 | −0.234 | −0.759 | −0.305 | −0.035 | −0.083 |
| LOC20189 | 1.2055 | 1.3605 | 0.2115 | 0.0375 | 2.8235 | 1.0265 | 0.2305 | 2.8275 | 0.5295 | 0.2585 |
| LOC25398 | 0.094 | −0.187 | −0.424 | −0.999 | −0.463 | −0.507 | −0.419 | −0.226 | 0.154 | −0.104 |
| LOC33990 | −0.85 | −0.594 | 0.687 | 0.926 | −0.661 | 0.35 | 0.237 | 0.007 | 0.181 | −0.088 |
| LOC34817 | −0.0205 | 0.2825 | 1.2335 | 0.5215 | 0.0675 | −0.2735 | 0.4465 | 9.0235 | 0.4125 | −0.6345 |
| LOC40045 | −0.51 | 0.28 | −0.18 | −0.241 | −0.287 | 0.199 | 0.478 | 0.211 | 0.57 | 1.126 |
| LOC44089 | −0.524 | −0.564 | 0.264 | 0.271 | −0.258 | −0.106 | −0.409 | −0.422 | −0.255 | −0.289 |
| LOC49386 | 0.102 | −0.319 | 0.696 | 0.004 | −0.089 | 0.407 | 0.149 | 0.395 | 0.875 | −0.003 |
| LOC51123 | −0.235 | −0.268 | 0.068 | −0.253 | 0.338 | −0.005 | 0.177 | 0.137 | −0.114 | −0.228 |
| LOC51161 | −0.9935 | −0.7875 | 1.2865 | 1.4345 | 1.2215 | 0.9605 | 0.6635 | 0.0965 | 0.7285 | −0.1135 |
| LOC51321 | 0.5015 | 0.6805 | −0.1005 | 0.4615 | 0.1595 | 0.5505 | 0.2175 | 0.2785 | 0.4125 | 0.3705 |
| LOC51760 | 0.5145 | 0.8195 | −0.9775 | −0.7275 | −0.5005 | −0.0195 | −0.9805 | −0.5865 | −0.0145 | 0.2865 |
| LOC81558 | −0.591 | −1.703 | 0.434 | 1.331 | −0.326 | −0.22 | 0.495 | 0.379 | 0.93 | −0.584 |
| LOC81569 | −0.0295 | −0.1525 | 0.1045 | −0.8365 | −1.2885 | 0.2325 | −0.3375 | −0.0595 | −0.1515 | −0.0835 |
| LOC90355 | −0.47 | −1.395 | −0.383 | 0.5 | −0.101 | −0.612 | −0.317 | 0.22 | −0.858 | −0.545 |
| LOC90701 | −0.109 | 0.994 | 0.952 | 0.861 | 0.649 | −0.947 | 0.408 | −2.277 | −1.679 | 1.689 |
| LONP | −0.1885 | −0.6825 | −0.0415 | −0.4815 | −0.5715 | −0.1295 | 0.4515 | 0.0425 | 0.1345 | −0.2705 |
| LRAP | −0.0035 | 0.5885 | 1.8535 | 1.0325 | 0.8155 | 0.7345 | 0.9775 | 0.6595 | 1.4875 | 0.3675 |
| LRBA | 0.5455 | 0.2575 | 0.4575 | 0.7405 | 0.8835 | −0.4515 | 0.2925 | 0.2515 | 0.0845 | −0.1985 |
| LRIG1 | −0.7345 | 1.2215 | −0.3615 | −1.6115 | −2.1665 | −0.5715 | −1.8925 | −1.5085 | −0.8265 | −1.0315 |
| LRP6 | 0.487 | 0.472 | 0.967 | 1.155 | 1.131 | −0.206 | 1.008 | 0.65 | 1.508 | 1.989 |
| LRP8 | −0.515 | −0.712 | 0.516 | 1.884 | 0.258 | 0.235 | 0.744 | 0.579 | 1.107 | −0.194 |
| LRRC17 | −0.2105 | −0.3835 | −0.4095 | −0.1975 | −0.2625 | 0.3565 | −0.2125 | −0.1285 | −0.1095 | 0.0635 |
| LRRC2 | −0.5445 | −0.4345 | 0.2805 | −0.0405 | −0.1885 | −0.1085 | −0.1705 | −0.2385 | −0.1895 | −0.1065 |
| LSM1 | −1.3835 | −1.3395 | 0.5405 | 0.9035 | −1.3085 | −0.8315 | 1.6745 | −0.6135 | −0.2095 | −0.0035 |
| LSM8 | 0.082 | 0.457 | 0.539 | 0.268 | −0.341 | −0.605 | −0.337 | −0.352 | −0.075 | −0.103 |
| LTB | −0.561 | 0.036 | 1.048 | 0.399 | −0.204 | 0.304 | 0.198 | −0.524 | −0.152 | 0.931 |
| LY6D | −0.603 | −0.095 | −0.327 | −1.195 | −1.435 | −0.575 | −0.385 | 0.185 | −0.583 | −1.495 |
| LYN | −0.228 | −0.232 | 0.17 | 0.291 | 0.188 | −0.14 | −0.053 | 0.222 | 0.022 | −0.198 |
| MA02L1 | 0.602 | 1.744 | −0.593 | −0.259 | −0.189 | 0.24 | −0.299 | −0.193 | 0.273 | 0.305 |
| MADP-1 | 0.6945 | 0.4035 | −2.0945 | −0.5225 | −0.6175 | 0.3235 | −0.6345 | −0.9505 | 0.1185 | −0.4205 |
| MAGEA12 | 0.5 | 2.04 | −0.7 | −0.318 | −0.298 | 0.308 | −0.458 | −0.491 | 0.11 | 0.053 |
| MAGEA2 | 0.8575 | 0.7235 | −1.1035 | −0.6555 | −1.5855 | 0.2955 | −0.8765 | −0.4995 | −0.5945 | −0.1755 |
| MAGEA3 | 0.8355 | 1.0755 | 0.2455 | −1.7915 | 0.2695 | −0.2595 | −0.9605 | 0.5095 | −0.1935 | −0.3255 |
| MAGEA4 | 0.423 | 0.005 | 0.607 | −0.005 | −1.263 | −0.133 | 0.471 | 0.309 | 0.072 | 1.039 |
| MAGED4 | −0.4365 | −0.9065 | −0.5535 | 0.1025 | −0.0445 | 0.0855 | 0.2395 | 0.4165 | −0.0025 | −0.4405 |
| MAGI1 | −1.4045 | −1.9705 | 0.4435 | 0.7335 | 0.0255 | −0.1685 | 0.0605 | −0.0035 | −0.2715 | −0.4785 |
| MAGI-3 | −0.617 | −0.166 | −0.009 | −0.566 | −0.42 | −0.234 | −0.321 | −0.411 | 0.081 | 0.125 |
| MAL2 | −0.953 | −0.64 | −1.208 | −1.559 | −1.117 | 0.85 | −1.059 | −0.423 | 0.095 | −0.495 |
| MAN2B1 | −0.7465 | −0.6605 | 0.1425 | −0.1035 | 1.3565 | −0.7115 | −0.0645 | −0.2785 | −0.1655 | −0.2745 |
| MAPK8IP2 | −0.2415 | −0.3525 | −0.4605 | 2.0625 | −0.2685 | −0.1505 | −0.8115 | −1.9835 | −0.5485 | 0.3205 |
| MARCO | 0.738 | −0.06 | 0.847 | −0.74 | −0.498 | 0.551 | 1.541 | 0.579 | 0.225 | 0.168 |
| MARLIN1 | −0.239 | −0.218 | −0.495 | −0.738 | −0.42 | −0.309 | −0.6 | −0.517 | −0.158 | −0.328 |
| MASS1 | −0.2655 | −0.2025 | −0.1005 | −0.0175 | −0.4875 | −0.5345 | −0.3435 | −0.2995 | −0.3425 | −0.5225 |
| MCM2 | 0.5875 | 0.9895 | 0.9725 | 1.7065 | 2.7855 | 1.0615 | −0.5385 | −0.2845 | −0.5935 | −0.8165 |
| MCM3 | −0.984 | −0.851 | 0.326 | 0.075 | −0.12 | 0.079 | −0.569 | −0.703 | 0.877 | −0.662 |
| MESP1 | −1.2645 | −1.5115 | 0.2959 | 0.0515 | 0.0495 | −0.8145 | −0.0135 | −1.2395 | 0.8275 | 1.1275 |
| METRN | 0.6565 | 0.4235 | 0.0295 | −0.0165 | −0.1865 | −0.2395 | −0.0065 | 0.1375 | 0.3365 | −0.0115 |
| MFAP2 | −0.364 | −0.437 | −0.285 | 0.176 | −0.001 | −0.546 | −0.102 | 0.071 | 0.106 | −0.688 |
| MFGE8 | −0.7825 | −0.3255 | −0.4275 | 0.0255 | −0.0485 | 0.0385 | −0.4125 | −0.4005 | −0.3535 | −0.4245 |
| MGAT4B | −0.271 | −0.136 | −0.271 | −0.04 | −0.692 | −0.596 | −0.054 | −0.785 | −0.312 | 0.172 |
| MGC1091 [symbol] | −0.9885 | −1.5055 | −0.0525 | 0.7055 | −0.6005 | −0.3395 | −0.5905 | 0.2935 | −0.5295 | −1.0145 |
| MGC1098 [symbol] | 0.5975 | 0.1355 | 0.3375 | −1.0905 | −0.3495 | −0.2605 | −0.4335 | 0.4385 | −0.0485 | −0.2415 |
| MGC1124 [symbol] | 0.035 | 0.555 | 0.192 | 0.135 | −0.089 | 0.291 | −0.368 | 0.069 | 0.242 | 0.21 |
| MGC1127 [symbol] | −0.008 | −0.103 | −0.227 | −0.251 | −0.56 | −0.177 | −0.177 | −0.598 | −0.826 | −0.491 |
| MGC1312 [symbol] | −0.384 | −0.193 | 0.431 | 0.733 | 0.247 | 0.125 | −0.102 | −0.264 | −0.258 | −0.69 |
| MGC1318 [symbol] | −0.209 | 0.038 | 0.373 | 0.671 | 0.688 | 0.8 | 0.09 | −0.133 | −0.128 | −0.142 |
| MGC1414 [symbol] | −0.8395 | 0.0105 | 0.1505 | −0.0825 | −0.0635 | −0.1845 | 0.1295 | −0.0275 | −0.2985 | −0.2405 |
| MGG1428 [symbol] | −0.436 | −0.674 | 2.10 | 0.043 | 0.659 | −0.507 | 0.674 | 0.017 | −0.316 | −0.356 |
| MGC1481 [symbol] | −0.372 | −0.722 | −0.623 | −0.648 | −0.938 | −0.225 | −0.558 | −0.477 | 0.705 | −0.544 |
| MGC1580 [symbol] | −0.1195 | −0.0435 | −0.0165 | 0.1685 | 0.1615 | −0.4345 | 0.4685 | −0.0816 | −0.1435 | −0.1035 |
| MGC1663 [symbol] | −0.251 | −0.129 | −0.17 | −0.081 | −0.159 | −0.638 | −0.229 | −0.41 | −0.373 | −0.389 |
| MGC1873 [symbol] | 0.043 | 0.11 | −0.429 | −0.436 | −0.852 | −0.34 | −0.95 | −0.49 | 0.969 | −0.044 |
| MGC2053 [symbol] | 0.038 | −0.397 | 1.219 | 1.887 | −0.158 | 0.568 | 1.477 | 0.782 | 0.544 | −0.285 |
| MGC2398 [symbol] | −0.051 | −0.254 | 0.201 | 0.264 | −0.9 | 0.114 | −0.14 | 0.051 | 0.228 | 0.188 |
| MGC2610 | 0.173 | −0.458 | 1.818 | 1.898 | −0.757 | 1.015 | 0.999 | 0.834 | −0.052 | 1.086 |
| MGC2714 | 0.161 | −0.814 | 1.976 | 1.346 | −0.738 | 0.927 | 1.458 | 0.331 | 0.389 | 0.647 |
| MGC2716 [symbol] | 0.3235 | −0.5215 | 1.7785 | 0.9575 | −0.6885 | 1.1055 | 1.1745 | 0.4465 | 0.3755 | 0.8685 |
| MGC2716 [symbol] | 0.1985 | −0.7915 | 2.4885 | 1.4705 | −0.5715 | 0.4755 | 0.4885 | 0.0735 | 0.3315 | 0.7785 |
| MGC2716 [symbol] | 0.179 | 0.083 | 1.821 | 0.769 | −1.326 | 1.67 | 0.838 | −0.171 | 0.28 | 1.556 |
| MGC2716 [symbol] | −0.0725 | −0.4055 | −0.0575 | 0.0565 | −0.1315 | −0.1155 | −0.2415 | −0.8005 | 0.1595 | −0.3995 |
| MGC2716 [symbol] | 0.003 | 0.282 | 0.985 | −0.171 | 0.735 | 0.772 | 0.799 | 1.25 | 0.946 | −0.178 |
| MGC2887 [symbol] | −0.412 | −0.354 | −0.304 | −0.493 | −0.047 | 0.083 | −0.175 | 0.239 | −0.305 | −0.399 |
| MGC3212 [symbol] | −0.539 | 0.848 | 1.4 | 1.278 | 0.252 | 0.521 | 0.461 | 0.644 | 0.374 | 0.035 |
| MGC3321 [symbol] | 0.7515 | 1.3995 | −0.6525 | −1.4755 | −0.6815 | 0.7305 | −1.1115 | −1.3355 | 0.1695 | −0.3625 |
| MGC3484 [symbol] | 0.5225 | 0.0135 | −0.2205 | 0.3435 | −0.3155 | 0.4645 | −0.0635 | −0.1195 | −0.3185 | −0.5835 |
| MGC3492 [symbol] | 0.282 | 0.282 | 0.818 | −0.247 | 0.176 | −0.001 | 0.297 | 0.277 | 0.373 | −0.215 |
| MGC4021 [symbol] | 0.075 | −0.164 | 0.337 | −0.529 | 0.054 | 0.052 | −0.331 | −0.171 | 0.541 | −0.304 |
| MGC4251 | −0.4925 | −0.4525 | 0.2145 | −0.2345 | −0.0025 | −0.1785 | −0.3995 | −0.7955 | −0.0855 | −0.1785 |
| MGC4308 | −0.183 | −0.466 | 0.723 | −0.262 | −0.011 | −0.007 | 0.383 | −0.082 | 0.538 | 0.119 |
| MGC4800 | 2.4845 | 2.3745 | 1.2845 | 1.2145 | −1.5345 | 0.2905 | 0.3315 | 3.2215 | 1.5345 | 1.9975 |
| MGC4659 | 0.709 | 1.053 | 0.466 | 1.15 | −0.137 | 0.462 | 0.061 | 0.208 | 0.15 | 0.316 |
| MIA | 1.0805 | 1.1835 | 0.6195 | 0.8825 | −0.0095 | 0.5835 | 0.1925 | 0.2895 | 0.2365 | 0.2965 |
| MID1 | 1.2045 | 1.3905 | 0.7875 | 0.8245 | −0.8085 | 0.9175 | −0.0035 | 0.7925 | 0.1415 | 0.4515 |
| MID1 | −1.654 | −1.463 | −0.991 | −1.291 | −1.285 | −1.6 | −1.075 | −0.496 | −0.855 | −1.449 |
| MID1 | −0.4105 | 0.9045 | 0.6815 | −0.7575 | −0.5725 | 1.1005 | −0.8625 | −0.8335 | −0.2915 | −0.4445 |
| MLF1IP | −0.9945 | 0.3425 | −0.6295 | −1.8625 | −1.2126 | 1.7715 | −1.6495 | −1.9785 | −0.3125 | −1.1905 |
| MLSTD1 | 0.451 | 0.557 | 0.456 | 0.61 | −2.093 | 0.026 | 0.257 | 0.914 | 0.431 | −0.253 |
| MMP1 | −0.169 | −0.057 | 0.74 | 0.186 | −0.336 | 0.341 | 0.205 | 0.298 | 0.13 | −0.106 |
| MMP7 | −0.4845 | −0.2755 | −0.3485 | 0.0005 | −0.3745 | −0.6416 | −0.1495 | −0.3855 | −0.1565 | −0.6245 |
| MNAT1 | −0.2545 | −0.4275 | 0.1515 | −0.4035 | −0.7285 | 0.1785 | −0.6705 | −0.6355 | −0.4205 | −0.4415 |
| MRPL14 | −0.188 | −0.008 | −0.028 | −1.122 | −0.661 | −0.06 | −0.493 | −0.339 | 0.068 | 0.118 |
| MRPL15 | −0.2045 | 0.0225 | 0.0755 | −0.0595 | 0.0995 | −0.1626 | 0.1555 | −0.4515 | 0.1735 | −0.3315 |
| MRPL27 | −0.4985 | −0.1175 | 0.2515 | −0.3825 | −0.1155 | 0.2385 | −0.3365 | 0.1065 | 0.1605 | −0.0125 |
| MRPL34 | 0.037 | 0.106 | 0.165 | −0.169 | 0.332 | 0.027 | 0.48 | 0.188 | −0.006 | −0.235 |
| MRPL43 | −0.0425 | −0.3015 | −0.1235 | −0.1175 | −0.1825 | −0.4645 | 0.0895 | −0.1305 | 0.1235 | −0.2395 |
| MRPL45 | −0.057 | −0.182 | −0.635 | −0.05 | 0.175 | −0.257 | −0.024 | −0.427 | 0.23 | −0.219 |
| MRPL48 | −0.8485 | −1.0575 | 0.0025 | 0.1915 | 0.4355 | −0.1215 | 0.0805 | −0.2375 | 0.1475 | −0.1475 |
| MRPL9 | −0.4905 | −0.3185 | −0.2805 | −0.0785 | 0.0905 | −0.0055 | −0.5305 | −0.3455 | −0.3065 | −0.2445 |
| MRPS14 | −1.245 | 0.222 | 0.156 | −0.031 | −0.012 | −0.545 | −0.581 | −0.539 | −0.476 | −0.694 |
| MRPS15 | −0.245 | 0.156 | −0.019 | −0.168 | −0.191 | −0.195 | −0.533 | −0.392 | −0.303 | −0.286 |
| MRPS17 | −0.145 | −0.304 | 0.245 | −0.35 | −0.122 | −0.293 | −0.733 | −0.555 | −0.033 | −0.472 |
| MRPS24 | 0.0285 | 0.0495 | 0.0985 | 0.5285 | 0.1285 | −0.0395 | 0.2755 | 0.2505 | 0.3475 | −0.1065 |
| MRPS25 | −0.514 | −0.861 | 0.248 | 0.124 | 0.296 | −0.382 | −0.026 | −0.137 | 0.326 | 0.028 |
| MRPS31 | −0.184 | −0.345 | 0.375 | 1.433 | −0.329 | 0.257 | 2.236 | −0.268 | −0.088 | −0.099 |
| MRPS6 | −0.2175 | −0.5735 | 0.0585 | −0.6215 | −0.5405 | −0.3225 | −0.1295 | −0.0515 | 0.1235 | −0.5895 |
| MS4A1 | 0.0705 | 0.6965 | 0.7635 | 0.3765 | 0.6545 | 0.5205 | 0.4865 | 0.2755 | −0.2175 | −0.3835 |
| MSH2 | 0.807 | −0.199 | 1.381 | −1.132 | −0.995 | −0.64 | 1.47 | −0.88 | −0.743 | 0.081 |
| MSH3 | −0.504 | −0.174 | −0.743 | 0.52 | 0.2 | 0.065 | 0.47 | 0.009 | 0.063 | 0.051 |
| MSMB | −0.433 | 0.344 | −0.017 | 0.002 | −0.531 | −0.068 | 0.32 | −0.002 | 0.226 | −0.368 |
| MTA1 | −0.2445 | −0.7195 | 0.4375 | 1.5075 | −0.2735 | 0.0025 | 0.9415 | 0.8475 | 0.5655 | 0.2405 |
| MTA3 | 0.315 | 0.213 | 0.034 | −0.412 | −0.168 | −0.355 | −0.406 | −0.317 | −0.285 | −0.137 |
| MTAC2D1 | 0.042 | −0.012 | 0.389 | −0.264 | 0.884 | 0.195 | 0.067 | 0.001 | 0.113 | −0.569 |
| MTCH1 | −0.2465 | −0.5425 | 0.1325 | 0.3495 | 0.3865 | −0.4705 | 0.0785 | 0.2475 | 0.3345 | 0.2975 |
| MTHFD1 | −1.5025 | −1.1475 | 0.1995 | 1.3535 | 1.2715 | 1.0445 | 0.6125 | 0.4965 | 1.2395 | 1.1615 |
| MTMR2 | −0.3985 | −0.0215 | 1.9965 | 0.3375 | 0.0465 | −0.0395 | −0.3345 | −0.5075 | −0.3235 | 0.2675 |
| MUC1 | 0.182 | 0.48 | 0.449 | 0.619 | 0.969 | −0.049 | 0.028 | −0.069 | −0.028 | 0.109 |
| MUC20 | 0.647 | 0.33 | 0.289 | 0.315 | 0.203 | 0.043 | 0.656 | 0.473 | 0.438 | 0.044 |
| MUM1 | −0.773 | −1.006 | 1.108 | 0.39 | −0.105 | −1.175 | 1.326 | 1.448 | 0.976 | −0.001 |
| MXI1 | 0.808 | −0.742 | −0.172 | −1.317 | −1.145 | −0.008 | −0.772 | −0.469 | −0.352 | −1.001 |
| MYB | −0.632 | −0.804 | −1.353 | −1.524 | −1.576 | −1.029 | −0.85 | −1.518 | −0.897 | −1.223 |
| MYBL1 | −0.745 | −0.268 | −0.552 | 0.344 | −0.380 | −0.839 | −0.115 | −0.9 | −0.557 | −0.801 |
| MYBL2 | −1.142 | −1.994 | −0.088 | 0.855 | 0.435 | −0.403 | 0.688 | 0.433 | 1.056 | −0.051 |
| MYO10 | −0.0365 | 0.3835 | 0.4045 | 0.3075 | −0.0555 | 0.0015 | 0.2315 | −0.1675 | −0.1625 | −0.2185 |
| MYO6 | −0.1795 | −0.8955 | 0.9945 | −1.8625 | −0.5325 | −1.4715 | −0.5465 | −2.0285 | −1.1685 | −1.0635 |
| MYST3 | −0.0655 | −0.4495 | −0.1255 | −0.2525 | −0.4105 | 0.9725 | 0.0235 | −0.0995 | 0.1035 | 0.1115 |
| NALP2 | −0.8495 | −0.6555 | 1.1255 | 0.0315 | −0.2595 | 0.7275 | −0.2305 | −0.1135 | −0.3195 | −0.0215 |
| NALP6 | −0.2225 | −0.1885 | −0.1355 | −0.8735 | −0.4125 | −0.1845 | −0.4755 | −0.5375 | −0.1815 | −0.2315 |
| NANS | −0.398 | −1.33 | −0.075 | 1.064 | 2.267 | 0.01 | 3.5 | 2.796 | 2.279 | 0.608 |
| NARF | 1.406 | 1.818 | −0.04 | 0.112 | −0.837 | −0.085 | 0.285 | 0.473 | 0.808 | 0.101 |
| NAT2 | −0.7515 | −0.5185 | −0.7845 | −0.9855 | −0.4185 | 0.0605 | −0.4535 | −1.0705 | 0.1295 | −0.4195 |
| NAV2 | −0.291 | −0.408 | 0.142 | −0.626 | 0.017 | 0.112 | −0.047 | 0.178 | −0.039 | 0.222 |
| NDE1 | 1.9635 | 2.7315 | 1.1875 | 0.4995 | 0.7645 | 0.9065 | 0.5055 | 0.5705 | 0.2305 | 0.4475 |
| NDP52 | −0.824 | −0.459 | 0.064 | 0.386 | 0.109 | −0.053 | −0.184 | −0.045 | −0.332 | −0.421 |
| NDRG2 | −0.667 | −0.52 | −0.24 | 0.373 | 0.406 | 0.086 | 0.259 | 0.229 | −0.403 | −0.308 |
| N0UFA7 | −0.3385 | −0.0785 | 0.3945 | 0.1795 | 0.3735 | −0.1075 | −0.2485 | −0.3305 | 0.0855 | −0.3365 |
| NDUFB1 | −0.734 | −0.68 | −0.044 | 0.174 | 0.258 | −0.119 | −0.364 | −0.048 | −0.412 | −0.376 |
| NDUFB10 | −0.157 | −0.932 | 0.293 | 0.212 | 0.231 | 0.385 | 0 | 0.263 | 0.079 | −0.382 |
| NDUFB2 | −0.3325 | −0.4155 | 0.1145 | 0.2445 | −0.0555 | 0.3695 | −0.1585 | 0.2385 | −0.3225 | −0.5055 |
| NDUFB5 | −0.4905 | −0.5505 | −0.1935 | −0.0525 | −0.4215 | −0.2985 | 0.0305 | −0.1775 | −0.0155 | −0.3475 |
| NDUFB6 | −0.6735 | −1.1275 | −0.0175 | −0.2355 | −0.4295 | −0.6375 | −0.4455 | −0.2705 | −0.7895 | −0.7476 |
| NDUFC2 | −1.3665 | 1.0045 | −1.3025 | −1.5435 | −0.8725 | −0.9935 | −0.3235 | −1.4725 | −1.1175 | −1.5045 |
| NEBL | −0.265 | −0.248 | 0.369 | −0.45 | −0.5 | 0.118 | −0.124 | 0.092 | −0.09 | 0.032 |
| NEK2 | −0.2 | −0.201 | 0.259 | −1.12 | 2.355 | −0.391 | 0.429 | −0.165 | −0.361 | −0.517 |
| NEK6 | −0.2515 | 1.3005 | 0.4145 | −0.0015 | 0.0885 | 0.3825 | −0.8315 | 0.3915 | 1.1695 | 0.1425 |
| NELL2 | −0.5055 | 0.3745 | 0.1145 | −0.6785 | −0.3515 | −0.1735 | −0.4585 | −0.9595 | 0.8045 | −0.4075 |
| NEURL | −0.012 | 0.268 | 0.197 | 1.976 | 1.287 | 0.888 | 1.053 | 0.819 | 0.543 | 0.268 |
| NFE2L3 | 0.6705 | 1.6425 | 0.5885 | 1.7855 | 0.9495 | 0.0125 | 0.5575 | 0.9955 | 0.5335 | 0.1605 |
| NFIA | −0.552 | −0.01 | −0.004 | 1.149 | 0.808 | −0.095 | −0.177 | 0.458 | 0.165 | −0.901 |
| NFIB | −0.2825 | −1.0755 | 0.3275 | 0.3755 | 0.2345 | 0.4825 | 0.1575 | 0.9145 | 0.2905 | −0.0865 |
| NINJ1 | 0.0335 | 0.8765 | −0.3005 | 0.2725 | −0.2595 | 0.1365 | −0.0875 | −0.0165 | −0.1635 | −0.0205 |
| NIPSNAP [symbol] | 0.2225 | 0.3115 | −0.2195 | −0.9005 | −0.5155 | −0.0045 | −0.3775 | −0.3245 | 0.1925 | −0.1645 |
| NKX8-2 | 0.1995 | 0.2565 | 0.0905 | −1.0195 | −0.3965 | −0.2625 | −0.7155 | −0.1035 | 0.3455 | 0.0265 |
| NME1 | −0.534 | −0.248 | −0.14 | 0.199 | 0.679 | 0.275 | −0.167 | −0.153 | 0.32 | −0.025 |
| NME2 | −1.7568 | −1.8125 | 1.1495 | −0.1605 | 0.7175 | 1.1775 | 1.3335 | 2.7185 | 0.1345 | −0.6075 |
| NME3 | −0.3895 | −1.0395 | 0.0605 | −0.2825 | 0.0025 | −0.1095 | −0.2835 | −0.0315 | −0.0995 | −0.0485 |
| NMES1 | −0.361 | −0.382 | −0.125 | −0.405 | −0.503 | −0.594 | −0.608 | −0.024 | −0.448 | −0.097 |
| NMI | 0.1305 | 0.3705 | −0.0915 | −0.3625 | −0.1585 | −0.1835 | −0.4605 | −0.3945 | 0.1295 | 0.0545 |
| NOC4 | −0.2515 | −0.3665 | 1.7695 | 1.4595 | −0.4085 | 0.3685 | 0.0785 | −0.0395 | 0.3105 | 1.1915 |
| N0LC1 | 0.1515 | −0.3405 | 0.5085 | −0.1515 | 0.4055 | 0.7465 | 0.4915 | 0.2095 | 0.2755 | 0.2975 |
| NotFound | 0.3285 | −0.0495 | 0.7115 | −0.4385 | −0.5015 | 0.2445 | −0.4035 | −0.0885 | 0.1855 | −0.3245 |
| NotFound | −0.018 | −0.349 | 0.887 | 0.913 | −0.819 | 0.586 | 0.089 | −0.274 | −0.107 | 0.839 |
| NotFound | −0.434 | 0.469 | 0.009 | 1.264 | 0.324 | −0.713 | 0.383 | 1.219 | 0.174 | −0.564 |
| NotFound | −0.507 | 0.016 | 0.662 | 1.229 | −0.326 | 0.242 | 1.597 | 0.05 | −0.008 | 0.32 |
| NotFound | −0.6775 | −1.1515 | −0.1065 | −0.4805 | −1.5895 | 0.4805 | −0.1185 | −0.1615 | −0.0225 | 0.3365 |
| NotFound | 0.834 | 0.869 | 0.283 | 1.345 | −0.371 | −0.277 | 0.617 | 3.49 | 0.964 | 0.17 |
| NotFound | −1.1875 | 0.5465 | −1.2595 | −0.2425 | −1.6445 | −0.7105 | −1.1935 | −1.2955 | −1.9995 | −1.5655 |
| NotFound | −0.298 | −0.336 | 0.553 | 0.684 | −0.053 | 0.734 | 0.089 | −0.053 | −0.23 | 0.103 |
| NotFound | 0.7065 | 1.0925 | −0.0505 | −0.2045 | 0.9295 | 0.7555 | −0.2605 | 0.0495 | 0.1035 | −0.0105 |
| NotFound | 0.2615 | −1.0355 | 1.2775 | 1.7515 | −1.4895 | −0.0185 | 1.0545 | 0.5185 | 0.4695 | 1.0765 |
| NotFound | −0.0555 | −0.3025 | 0.2075 | −0.4545 | −0.1505 | 0.2345 | −0.5225 | −0.7295 | −0.3845 | −0.2365 |
| NotFound | −0.2715 | 0.1235 | −0.1005 | 0.1045 | 0.2685 | −0.6595 | −0.0875 | −0.2325 | −0.1275 | 0.0375 |
| NotFound | 0.471 | 0.475 | 0.382 | 0.065 | 0.285 | −1.095 | 0.532 | 1.168 | 0.07 | −0.827 |
| NotFound | −0.106 | 0.125 | −0.082 | 0.211 | 0.207 | 0.033 | 0.15 | 0.078 | −0.241 | −0.353 |
| NotFound | −0.826 | −0.002 | −0.128 | −0.383 | 0.177 | −0.089 | −0.434 | 0.502 | −0.152 | 0.09 |
| NotFound | 0.0085 | −0.0985 | 0.5625 | 0.2865 | −0.2135 | −0.0165 | −0.0805 | 0.1765 | −0.4155 | 0.2385 |
| NotFound | −0.368 | −0.328 | 0.008 | 0.143 | −0.293 | 0.302 | 0.093 | −0.002 | 0.317 | −0.046 |
| NotFound | 0.842 | 0.585 | 0.53 | 0.502 | 0.537 | 0.566 | 0.381 | 0.657 | 0.173 | −0.122 |
| NotFound | −0.4955 | −0.6845 | −0.4855 | 0.9185 | −0.3915 | −0.2385 | −0.3735 | −0.0315 | −0.0545 | 0.0085 |
| NotFound | 0.4646 | 0.8505 | 1.5885 | 2.0555 | 3.1615 | 2.6295 | 1.0125 | 1.4135 | 0.5795 | −0.0125 |
| NotFound | 0.3 | 0.789 | 0.571 | 0.511 | 0.281 | 0.662 | 0.41 | 0.057 | 0.268 | 0.358 |
| NotFound | 0.6915 | −0.5425 | −0.1555 | 0.8105 | −0.0035 | 1.2925 | 0.0725 | −0.3565 | −0.2505 | −0.0865 |
| NotFound | −1.044 | −1.188 | −0.224 | 1.155 | 2.73 | 0.686 | 3.337 | 3.165 | 3.242 | 1.608 |
| NotFound | −0.865 | −0.76 | −1.057 | −0.572 | −0.774 | −0.026 | −0.265 | −0.652 | −0.872 | −1.346 |
| NotFound | −0.5305 | −0.3895 | 0.8925 | −0.2765 | 0.0465 | −0.7585 | −0.4455 | −0.4855 | −0.6875 | −0.4485 |
| NotFound | −0.25 | −0.458 | −0.734 | 0.205 | 0.737 | −0.424 | 0.533 | −0.871 | 0.334 | −0.318 |
| NotFound | −0.9465 | −0.5125 | −1.2135 | −0.4005 | −0.7915 | −0.3525 | −0.4275 | −1.0565 | −1.0415 | −1.6575 |
| NotFound | 0.481 | 1.365 | −0.092 | 1.03 | −0.615 | 0.833 | −0.034 | −0.724 | −0.32 | 0.138 |
| NotFound | 0.87 | 0.101 | 0.973 | 0.322 | 0.473 | 0.022 | 0.794 | 0.959 | 0.495 | −0.07 |
| NotFound | −0.0765 | −0.4005 | 1.6065 | 0.7115 | −0.6555 | 1.1405 | 0.3775 | −0.8765 | −0.0185 | 0.9865 |
| NotFound | −0.5065 | −0.2455 | −0.2425 | −0.5755 | −0.7105 | −0.3115 | −0.6135 | −0.2105 | −0.6255 | −1.5475 |
| NotFound | −1.3815 | −0.1695 | −0.8335 | −0.1845 | −0.6785 | 0.2885 | −0.8705 | −1.1575 | −1.2835 | −1.3245 |
| NotFound | −0.9076 | 0.1825 | −1.0385 | −0.4755 | −1.1815 | 0.2755 | −0.4875 | −1.0055 | −0.9765 | −1.3865 |
| NotFound | −0.3845 | −0.7465 | −0.8875 | 0.9895 | −0.9955 | 0.6405 | 0.8745 | 0.9095 | 0.4185 | 0.2855 |
| NotFound | −0.697 | 0.337 | −1.112 | 0.255 | −0.525 | −0.326 | −0.724 | −0.381 | −1.032 | −1.17 |
| NotFound | −0.3145 | −1.1745 | −0.6095 | 0.5455 | 0.2245 | −0.4365 | 0.4125 | −0.4525 | −0.3925 | −1.3175 |
| NotFound | −0.3525 | −0.4045 | −0.4375 | −1.0935 | −0.8135 | −0.4625 | −1.0235 | −0.2685 | −0.4575 | −0.3715 |
| NotFound | −1.8205 | −0.5695 | −1.7385 | −0.1045 | −1.4785 | 0.3715 | −0.5355 | −1.3795 | −1.7255 | −0.8755 |
| NotFound | −0.2305 | −0.9995 | 0.1875 | 0.5365 | 0.5405 | −1.0345 | 0.0275 | 0.3255 | −0.8205 | −0.4365 |
| NotFound | 0.374 | 1.591 | −0.281 | −0.525 | 0.132 | 0.348 | −0.003 | −0.645 | −0.688 | −0.212 |
| NotFound | −0.053 | 0.228 | −0.262 | 0.158 | −0.016 | −0.008 | 0.179 | 0.27 | −0.162 | 0.045 |
| NotFound | 0.59 | −0.388 | 0.724 | 1.121 | −0.458 | 0.931 | 1.163 | 0.209 | 0.4 | 0.62 |
| NotFound | −0.8775 | −0.0825 | −0.8655 | −0.1725 | −0.6515 | −0.0455 | −0.2335 | −0.7995 | −1.0745 | −1.3445 |
| NotFound | −0.3045 | −1.8625 | 0.5825 | 0.4645 | −0.2085 | −0.4205 | 0.6125 | −0.7945 | 0.0755 | −0.2135 |
| NotFound | 0.056 | 0.08 | −0.095 | 0.379 | 0.098 | −0.366 | 0.021 | −0.327 | −0.191 | 0.236 |
| NotFound | −0.1615 | −0.0935 | −0.3995 | −0.0565 | −0.3135 | 0.1365 | 0.0515 | −0.2465 | −0.0315 | 0.2795 |
| NotFound | 0.0395 | −0.2445 | 0.6105 | −0.7085 | −0.9095 | −0.4405 | 1.3085 | −0.0055 | 0.9355 | 0.0895 |
| NotFound | 0.0335 | 0.8305 | 0.0505 | −0.3415 | 0.1145 | 0.1965 | −0.3615 | 0.0055 | 0.1375 | 0.2655 |
| NotFound | −0.5085 | −0.3175 | −0.2595 | 1.4455 | −0.0315 | 0.4345 | −0.3555 | −0.1275 | −0.1305 | −0.0915 |
| NotFound | 0.775 | 0.993 | 0.049 | 0.253 | 0.224 | −0.617 | 0.703 | 0.57 | 0.357 | −0.692 |
| NotFound | −0.035 | 0.172 | −0.384 | 0.707 | 0.157 | −0.16 | 0.636 | −0.32 | 0.559 | −0.665 |
| NotFound | −0.5375 | 0.0905 | 0.0125 | 1.3815 | 0.2445 | −0.8575 | 0.4125 | 0.8805 | 0.1765 | −0.5575 |
| NotFound | −0.2255 | −0.0505 | 0.1915 | −0.3725 | −0.1285 | 0.1105 | 0.1175 | 0.1335 | 0.4215 | −0.1835 |
| NotFound | −0.3405 | −0.2825 | 0.6675 | 0.3195 | 0.4475 | −0.9725 | 0.9955 | 0.9045 | −0.5905 | −0.2595 |
| NotFound | −0.1715 | −0.1435 | 0.4555 | 0.7605 | 0.4745 | 1.1535 | 0.4845 | 0.6305 | 1.3255 | −0.1825 |
| NotFound | 0.395 | 2.428 | −0.863 | −0.353 | 1.681 | 1.524 | −1.055 | 0.67 | 1.713 | 1.14 |
| NotFound | 1.11 | 0.736 | 1.398 | 1.545 | 2.682 | 2.198 | 1.441 | 1.295 | 1.032 | 1.358 |
| NotFound | −0.1065 | −0.6105 | 0.5545 | −0.0125 | 0.0505 | −0.2325 | 0.1455 | −0.1305 | −0.1065 | 0.0785 |
| NotFound | −0.045 | 0.906 | −0.112 | 2.199 | 0.753 | 1.505 | −0.171 | −0.567 | −0.735 | 0.517 |
| NotFound | −0.039 | 0.082 | 0.724 | −0.616 | −0.153 | 0.458 | −0.205 | 0.113 | −0.036 | 0.301 |
| NotFound | −0.9405 | −0.5205 | −0.0805 | 0.2995 | 0.5685 | 0.1055 | −0.1465 | 0.3715 | −0.6695 | 0.7445 |
| NotFound | 0.095 | 1.127 | −0.081 | 0.411 | 0.141 | 0.075 | 0.54 | −0.271 | 0.162 | −0.208 |
| NotFound | −2.052 | −0.541 | −1.031 | −0.128 | −0.839 | 0.211 | −1.331 | −1.74 | −1.847 | −1.435 |
| NotFound | −0.3695 | 0.5565 | 0.2055 | −1.3825 | −1.1355 | −0.0815 | −1.1175 | −0.2905 | −0.7865 | −0.5895 |
| NotFound | −0.542 | −0.163 | 0.865 | 0.782 | 0.488 | 0.077 | 0.381 | −0.113 | −0.001 | −0.071 |
| NotFound | −0.1445 | −0.0175 | 0.0145 | −0.6105 | −0.5715 | 0.0185 | −0.9765 | −0.6375 | −0.5025 | −0.2955 |
| NotFound | 0.1565 | 0.1515 | −0.3915 | −0.3225 | −0.2155 | −0.3855 | −0.2115 | −0.2525 | −0.4015 | −0.1035 |
| NotFound | −0.876 | −0.307 | −1.222 | −0.487 | −0.803 | −0.278 | −0.37 | −0.996 | −1.188 | −1.32 |
| NotFound | −0.8225 | −0.5525 | −0.8145 | −0.5555 | −0.4185 | −0.1035 | −0.6875 | −0.8525 | −0.1225 | −0.3955 |
| NotFound | −0.228 | 0.175 | 0.456 | −0.154 | 0.422 | 0.804 | −0.354 | −0.131 | −0.257 | 0.324 |
| NotFound | 0.258 | −0.285 | 0.204 | −0.229 | 0.122 | 0.109 | 0.544 | −0.108 | 0.11 | −0.642 |
| NPDC1 | 0.3185 | −0.1116 | −0.6765 | −0.2075 | −0.1345 | 0.1695 | −0.2385 | −0.4745 | 0.1555 | 0.2995 |
| NPEPPS | −0.589 | −0.82 | −0.294 | 0.682 | 0.423 | 0.38 | 0.72 | 0.199 | 0.38 | −0.339 |
| NPY | 0.224 | −0.086 | −0.148 | −0.213 | −0.097 | −0.138 | −0.733 | −0.914 | −0.308 | 0.006 |
| NQO3A2 | −0.922 | −1.213 | 0.614 | 0.561 | 0.418 | 0.03 | 1.439 | 1.226 | 1.262 | −0.044 |
| NRAS | 0.619 | 0.239 | 1.539 | 1.738 | 1.733 | 0.481 | 1.339 | 1.432 | 1.945 | 0.984 |
| NRIP1 | −0.2925 | −0.7075 | 1.3485 | 0.8935 | −0.1635 | 0.2265 | 0.3465 | 0.5725 | −0.1165 | 0.3795 |
| NTN4 | −0.6385 | −0.7675 | −0.5285 | −0.2785 | −0.0395 | −0.7205 | −0.2975 | −0.5275 | −0.3945 | −0.6575 |
| NUCB2 | 0.0735 | 0.2335 | 0.2535 | 0.5895 | 0.5975 | 1.2245 | 0.5095 | 0.5945 | 0.9775 | −0.5115 |
| NUDT5 | −0.784 | −0.612 | −0.006 | −0.816 | −0.921 | −1.208 | −0.185 | −0.142 | −0.145 | −0.74 |
| NUDT6 | 0.454 | 0.805 | 0.224 | −0.355 | −0.243 | 0.078 | 0.003 | 0.089 | −0.212 | −0.747 |
| OCIAD2 | 0.4105 | −0.3295 | −0.2135 | −0.0575 | −0.1205 | −0.5025 | −0.4005 | 0.3455 | 0.0145 | −0.2035 |
| OFD1 | 0.0045 | 0.2755 | 0.5165 | 0.1685 | −0.0345 | 0.0345 | −0.4095 | 0.2735 | −0.0305 | −0.5265 |
| OGFRL1 | 0.049 | 0.004 | −0.015 | −0.03 | 0.518 | −0.181 | −0.029 | −0.208 | 0.095 | 0.148 |
| OPLAH | −0.6485 | −0.6575 | 0.0585 | −0.4375 | −0.8265 | −0.3915 | −0.3335 | −0.7435 | −0.7595 | −0.1785 |
| ORMDL3 | −0.273 | −0.461 | 0.069 | 0.882 | 2.075 | 0.795 | 0.217 | 0.43 | −0.217 | 0.333 |
| OSBPL3 | −0.533 | −0.651 | 0.396 | −0.591 | −0.583 | −0.14 | −0.339 | −0.436 | −0.273 | 0.002 |
| OSR2 | −0.1405 | −0.4635 | 0.6385 | 0.5465 | −0.8425 | 0.3055 | 0.0345 | 0.2135 | −0.3085 | 0.2165 |
| OSTF1 | −1.113 | −1.256 | −0.988 | 0.594 | 0.281 | −0.196 | 0.438 | 0.632 | 0.364 | 0.247 |
| P2RX1 | 0.5135 | 0.9285 | 1.0885 | 0.2125 | −0.9335 | 1.0165 | −0.2275 | −0.0815 | −0.3055 | 0.4885 |
| P2RY2 | 0.263 | 0.748 | −0.206 | 0.623 | −0.616 | −0.453 | −0.242 | −0.385 | 0.054 | −0.389 |
| PACAP | −0.28 | 0.237 | −0.323 | −0.391 | −0.825 | 0.046 | −0.658 | −0.386 | −0.64 | −0.291 |
| PADI2 | −1.0025 | −0.5565 | 1.4945 | 0.4235 | −0.0485 | 1.1005 | −0.4075 | −0.5525 | 0.2855 | 0.2335 |
| PAPD5 | −0.071 | 0.16 | −0.618 | −0.271 | −0.289 | −0.291 | −0.315 | −0.571 | −0.156 | −0.3 |
| PAPSS2 | −0.914 | −1.179 | −0.724 | −0.006 | −0.245 | −0.71 | −0.385 | −0.91 | −0.102 | −0.162 |
| PARD8A | −0.806 | −1.48 | 0.149 | −1.031 | −0.68 | 0.197 | −0.239 | 0.001 | 0.138 | −0.495 |
| PARP1 | −0.49 | −0.161 | −0.421 | 0 | 0.103 | 0.041 | −0.055 | 0.163 | −0.105 | −0.654 |
| PARP9 | −0.2205 | 0.2255 | −0.2865 | −0.0285 | 0.0935 | −0.0975 | −0.2945 | 0.2555 | 0.2325 | 0.0055 |
| PAXIP1L | 0.0225 | −0.0305 | −0.0185 | 0.3895 | 0.4405 | −0.1645 | −0.0135 | 0.4045 | −0.1175 | −0.1555 |
| PBX3 | −0.0145 | −0.6845 | −0.0805 | 0.1925 | 0.4095 | 0.2845 | 0.2355 | 0.3395 | 0.5815 | 0.5315 |
| PCDHB11 | 0.292 | −0.338 | −0.027 | 0.799 | 0.802 | 0.088 | −0.527 | 1.068 | −0.441 | −0.377 |
| PCDHB16 | 0.21 | −0.16 | 0.088 | 1.189 | 0.888 | 0.724 | 1.469 | 0.855 | 0.397 | 0.223 |
| PCDHB2 | −0.6495 | −0.8485 | −0.0205 | −0.6225 | −0.4765 | −0.5955 | −0.2885 | −0.2875 | −0.4035 | −0.4425 |
| PCM1 | −0.3805 | −0.5375 | 1.0845 | −0.1545 | −0.4035 | −0.2305 | −0.2655 | −0.4165 | −0.1255 | 0.1955 |
| PCNA | −0.546 | −1.528 | 0.227 | 0.005 | −0.284 | −0.093 | 0.341 | 0.35 | 0.421 | −0.573 |
| PDC01 | 0.6535 | 1.2675 | 0.1115 | 1.9375 | 1.0565 | −0.4585 | 1.1045 | 1.0545 | 1.0735 | 0.2385 |
| PDC010 | −0.145 | −0.58 | −0.326 | −0.276 | −0.227 | −0.419 | −0.507 | −0.442 | −0.126 | −0.271 |
| PDCD4 | −0.5735 | −1.0455 | −0.0205 | 0.0035 | −0.2555 | −0.5455 | −0.3025 | −0.2425 | −0.4015 | −0.5025 |
| PDCD5 | 0.2655 | 1.4465 | 1.1325 | −0.6265 | 0.5505 | −0.5355 | −1.3335 | 0.1025 | 1.0605 | 1.2185 |
| PDCD8 | −0.1005 | −0.2175 | −0.0665 | 0.1325 | −0.0915 | −0.0245 | −0.4885 | −0.1445 | −0.1935 | −0.2825 |
| PDE4DIP | −0.0435 | −0.3005 | 0.1025 | 1.1105 | 0.7625 | 0.4835 | 0.7745 | 0.8885 | 0.1895 | −0.0375 |
| PDHA1 | −0.135 | 0.529 | −2.755 | −1.834 | −0.956 | −1.117 | −0.356 | 2.841 | 0.441 | −0.183 |
| PECI | 0.0745 | 1.4285 | 0.3895 | 0.9476 | 1.0285 | 1.1355 | 0.3315 | 1.2235 | 0.9805 | 1.0695 |
| PEG10 | 0.2005 | 0.3755 | 0.6175 | −0.4765 | −0.2435 | −0.2605 | 0.0245 | 0.8005 | 0.1735 | −0.2555 |
| PER2 | −0.274 | 0.263 | −0.052 | 0.440 | 0.379 | 0.743 | 0.396 | 0.075 | −0.491 | −0.522 |
| PERP | 0.6715 | 0.0325 | −0.1005 | 0.0545 | 0.1885 | 0.3485 | 0.6755 | 0.2575 | 0.4205 | 0.2245 |
| PEX1 | −0.487 | −0.249 | −0.525 | −0.3 | 0.185 | −0.276 | −0.182 | −0.373 | 0.121 | 0.067 |
| PFAAP5 | −0.1325 | 0.5625 | −0.5476 | 0.7825 | 0.9605 | 0.6415 | −0.6885 | 1.1065 | 0.7835 | −0.0615 |
| PFDN2 | 0.01 | −0.12 | −0.202 | −0.233 | −0.218 | 0.18 | −0.183 | 0.188 | 0.191 | 0.27 |
| PGM1 | −0.2015 | 0.1855 | 0.4025 | −0.0065 | −0.1675 | 0.1335 | −0.2045 | 0.3515 | 0.2175 | 0.0065 |
| PGR1 | 0.4405 | 0.8345 | −0.4415 | −0.0585 | −0.3095 | 0.3105 | −1.0475 | −0.7375 | 0.3295 | −0.4045 |
| PHAX | −0.85 | −0.515 | 0.383 | 0.591 | 0.602 | 0.215 | 0.42 | −0.288 | −0.487 | −0.333 |
| PHGDH | −0.7565 | −1.1195 | 1.2785 | 1.1975 | −0.5725 | 0.5225 | 0.7425 | 0.8065 | 1.0065 | 0.0055 |
| PHYH | −0.6745 | −0.5555 | 0.4425 | 0.4755 | −0.2685 | −0.09895 | 0.2715 | 0.1145 | 0.2005 | −0.2515 |
| PI85PA | 0.0325 | 1.0535 | 1.4845 | 0.5085 | −0.5595 | 1.1075 | 0.4595 | −0.5585 | 0.3395 | 0.9675 |
| PIGT | 0.126 | −0.238 | 0.128 | −0.374 | 0.089 | 0.218 | 0.56 | 0.133 | −0.093 | −0.025 |
| PIM2 | 0.2715 | 0.7725 | 0.7275 | −0.7365 | 0.0865 | 0.5985 | −0.4315 | −0.4755 | −0.3235 | −0.3385 |
| PISD | 0.414 | −0.383 | 0.452 | −1.414 | −3.888 | −0.864 | 1.327 | 1.495 | 0.91 | −0.082 |
| PITPNC1 | −1.2505 | −1.0935 | −0.3105 | −1.2485 | −0.7905 | −0.7965 | −0.8375 | −1.2115 | −0.8155 | −1.3235 |
| PKIB | −0.256 | −0.088 | 0.153 | 0.179 | 0.44 | 0.128 | −0.061 | 0.706 | 0.056 | −0.079 |
| PKMYT1 | 0.62 | −0.128 | 0.592 | −0.871 | −0.98 | −0.418 | 0.33 | −0.769 | 1.157 | −0.373 |
| PLA2G4B | 0.4975 | 0.9355 | 0.3455 | 0.1925 | 0.4745 | 0.1215 | −0.0955 | 1.4895 | −0.5385 | −0.5455 |
| PLAC8 | 0.53 | 0.02 | −0.62 | −0.276 | −0.151 | 0.496 | −0.683 | −0.429 | −0.18 | −0.954 |
| PLCB1 | 0.0525 | −0.4335 | 0.7205 | −0.1485 | −0.3175 | −0.0275 | −0.2545 | −0.2375 | 0.1395 | −0.2355 |
| PLCXD1 | 0.309 | 0.685 | −0.215 | −0.071 | −0.258 | 0.126 | −0.495 | −0.377 | −0.04 | 0.212 |
| PLEK2 | −0.2655 | −0.5285 | −0.3025 | −0.3325 | −0.2085 | 0.0035 | 0.0865 | −0.5815 | −0.1405 | 0.0015 |
| PLO0 | −0.0815 | −0.2045 | 0.3405 | 1.1865 | 1.6915 | −0.1655 | 0.2315 | 0.3515 | 0.6075 | −0.0545 |
| PNAS-4 | 0.3715 | −0.0765 | −0.6215 | 0.1235 | −0.4145 | 0.0915 | −0.3085 | 0.7985 | 0.2835 | 0.5075 |
| PNLIPRP2 | −0.8225 | −0.9505 | 0.5805 | −0.2625 | −0.9155 | 0.5605 | 0.1855 | 0.1175 | −0.2805 | −0.8355 |
| PNMA3 | 0.1695 | 0.3795 | −0.1525 | −0.3025 | −0.0295 | 0.0715 | 0.2385 | −0.0355 | 0.0535 | −0.0925 |
| POLB | −0.491 | −0.295 | 0.01 | −0.286 | −0.202 | −0.125 | −0.385 | −0.497 | −0.184 | −0.363 |
| POLR2F | −0.4325 | −0.1775 | −0.4135 | 0.1185 | 0.6455 | −0.0205 | 0.2565 | 0.6785 | −0.3175 | −0.2195 |
| POLR2H | −0.4805 | 0.3645 | 0.1095 | −0.0305 | −0.3975 | 0.1895 | −0.0255 | 0.0025 | 0.0245 | −0.5045 |
| POMC | −0.33 | −0.494 | −0.111 | −0.452 | 0.065 | −0.454 | −0.579 | −0.283 | −0.402 | −0.3 |
| POP4 | −0.89 | −0.291 | 0.431 | 0.538 | 0.254 | 0.05 | −0.238 | 0.009 | 0.278 | 0.155 |
| POP7 | −0.407 | −0.416 | −0.434 | −0.382 | −0.489 | −0.235 | −0.608 | −0.383 | −0.364 | −0.086 |
| PP1057 | −0.1235 | −0.3285 | 0.3505 | 1.5575 | 0.5845 | −0.5425 | 0.5405 | −0.1925 | 0.5245 | −0.3715 |
| PPFIA1 | −0.562 | −0.86 | 0.438 | −0.524 | 0.182 | −0.308 | −0.670 | −0.518 | −0.379 | −0.09 |
| PPFIBP2 | −0.3855 | −0.0215 | 0.1135 | −0.1765 | −0.4125 | −0.1475 | 0.0965 | 0.2385 | 0.1275 | −0.1736 |
| PPGB | −0.5985 | −0.4705 | 0.1005 | −0.4625 | 0.7415 | −0.7725 | −0.9755 | 0.2815 | 0.1735 | −0.5395 |
| PPHLN1 | −0.5405 | −0.2925 | −0.0405 | −0.2955 | −0.6665 | −0.4745 | −0.4235 | 0.3375 | −0.3225 | −0.5785 |
| PPIF | 0.1095 | 0.1085 | −0.3875 | 0.0645 | 0.1955 | 0.3755 | 0.0605 | −0.0165 | 0.5175 | 0.3175 |
| PPIL1 | −0.0715 | 0.1576 | −0.4945 | −0.2635 | −0.2865 | 0.1985 | −0.0485 | −0.3185 | −0.2375 | −0.5415 |
| PPOX | −0.04 | −0.318 | 3.473 | 1.055 | 2.042 | 0.343 | 1.187 | 0.004 | 0.725 | 1.089 |
| PPP1R16 [symbol] | −0.155 | 0.321 | −0.072 | −0.002 | 0.345 | 0.29 | 0.028 | −0.013 | −0.229 | −0.197 |
| PPP1R3C | −0.656 | −0.605 | −0.018 | 0.797 | 1.085 | 0.719 | 0.553 | 0.019 | 0.199 | 0.445 |
| PPP2R2D | −0.3325 | 0.9395 | 1.1065 | −0.6225 | −0.8935 | 0.6065 | −0.6935 | −0.8555 | 3.0385 | 0.1775 |
| PPP2R5A | −0.501 | −0.893 | −0.859 | −0.987 | −0.779 | −1.011 | −0.734 | −1.03 | −0.51 | −0.538 |
| PRAME | −0.4805 | −0.0195 | −0.2095 | −0.2055 | −0.6565 | −0.4905 | −0.0595 | −0.4075 | −0.1745 | −0.5305 |
| PRC1 | 0.097 | −0.32 | 0.18 | 0.45 | −0.251 | 2.187 | 0.638 | 0.456 | 0.109 | 0.282 |
| PRIM2A | 0.4725 | −0.5516 | 0.1005 | −0.0045 | −1.3815 | 0.5885 | 0.3405 | −0.5095 | −0.1375 | 0.4895 |
| PRKACB | −0.577 | −0.504 | 0.051 | 0.255 | 0.053 | −0.612 | −0.067 | −0.01 | 0 | −1.042 |
| PRKCB1 | 0.18 | −0.148 | −0.36 | 0.044 | −0.597 | −0.096 | 0.267 | 0.15 | 0.102 | 0.057 |
| PRKCI | −0.507 | −0.216 | −0.13 | −0.345 | 0.051 | −0.254 | −0.368 | −0.627 | −0.235 | −0.189 |
| PRKRIR | 0.4155 | −0.1245 | 0.4605 | −0.6805 | −0.2205 | −0.3445 | −0.2085 | −0.1205 | 0.1185 | −0.4045 |
| PRKWNK1 | 1.2535 | 0.6545 | 0.7175 | 0.3815 | 0.1435 | 0.2745 | 0.6075 | 1.4945 | 0.4755 | 0.4545 |
| PRKX | 0.037 | −0.308 | 0.405 | 0.879 | −0.084 | 0.451 | 0.511 | −0.189 | 0.637 | 0.564 |
| PRNP | −0.2745 | −0.8025 | 0.0545 | 0.0525 | −0.0015 | 0.3485 | −0.0135 | 0.1455 | 0.2495 | 0.1075 |
| PROL5 | 0.2766 | 0.2745 | −0.3635 | −0.5805 | 0.0845 | −0.4105 | 0.2005 | 0.2875 | −0.1175 | −0.5755 |
| PRPF18 | 0.7885 | 0.6085 | 0.2395 | −0.5355 | −0.5195 | 0.5795 | −0.0485 | 0.1195 | −0.1625 | 0.1956 |
| PRPSAP1 | 0.0095 | 0.0195 | 1.1485 | 0.2715 | −0.1295 | −0.8575 | 0.0185 | 1.0715 | 0.5785 | −0.2255 |
| PRSS12 | −0.4855 | −0.1415 | 0.3315 | 0.3255 | 1.3175 | 0.3205 | 2.0395 | 2.4825 | 1.6305 | −0.3235 |
| PRSS16 | −0.3155 | −0.7205 | 0.4475 | −0.2855 | −0.2725 | −0.0135 | −0.3565 | 0.2535 | −0.1805 | −0.2285 |
| PSD3 | −1.2335 | −1.1825 | −0.4115 | −0.7016 | −0.4335 | −0.4335 | −1.5765 | −1.0635 | −0.9785 | −0.7005 |
| PSMA3 | 0.13 | 0.324 | −0.234 | −0.551 | −0.084 | 0.257 | −0.107 | 0.043 | 0.028 | −0.009 |
| PSMA7 | −0.417 | −0.791 | 0.551 | −0.011 | −0.086 | 0.684 | 0.312 | 0.322 | 0.078 | −0.102 |
| PSMC5 | −0.3055 | −0.0695 | −0.0935 | −0.4735 | −0.8725 | 0.1365 | −0.4915 | 0.0005 | −0.0895 | −0.8085 |
| PSMC6 | −0.665 | −0.646 | −0.27 | −0.75 | −0.41 | −0.084 | −0.296 | −0.229 | −0.263 | −0.172 |
| PSMD12 | −0.3185 | 0.2545 | 0.0476 | −1.0305 | 0.4365 | −0.2075 | −0.6935 | 0.0225 | 0.0185 | −0.4535 |
| PSMD12 | 0.7655 | 0.2885 | 0.1025 | −1.8835 | −1.0225 | 0.5325 | 1.1825 | 0.5465 | −1.1295 | 1.1685 |
| PSMD3 | −0.0005 | 0.1725 | 0.7845 | −0.1195 | 0.4545 | 0.6425 | 0.4395 | 0.5735 | 0.3705 | 0.0905 |
| PSPH | −0.336 | −0.561 | 0.582 | 1.515 | 1.597 | 0.295 | 1.262 | 0.91 | 1.451 | 0.877 |
| PTD012 | 1.19 | 1.243 | 0.202 | 0.138 | 0.044 | 1.837 | 0.456 | 3.54 | 2.25 | 1.033 |
| PTGER3 | −0.582 | −0.137 | −0.19 | 0.181 | 0.668 | −0.532 | 0.28 | 0.042 | −0.486 | −0.48 |
| PTHLH | −0.2125 | 0.2615 | 0.0225 | 0.5935 | 0.2465 | −0.0825 | 0.1335 | 0.1515 | −0.0515 | −0.5185 |
| PTK6 | 0.037 | 0.138 | 0.012 | −0.21 | −0.374 | 0.029 | −0.568 | −0.63 | −0.43 | 0.222 |
| PTPN4 | −0.724 | 0.132 | −0.317 | 0.612 | 0.978 | −0.019 | 0.048 | −0.035 | −0.049 | −0.836 |
| PTPN7 | 0.562 | −0.306 | 0.826 | −0.048 | −0.274 | −0.63 | −0.617 | −0.601 | −0.728 | 0.392 |
| PTPRF | 0.009 | −0.272 | 0.617 | −0.324 | −0.197 | −0.364 | 1.174 | 0.629 | 1.394 | 0.312 |
| PTPRN2 | −0.4665 | −0.6695 | −0.0135 | 0.2485 | −0.3195 | −0.2305 | −0.1295 | 0.2225 | 0.3295 | 0.1595 |
| PTPRT | −0.6515 | −0.7455 | −0.8195 | −0.8585 | −0.9155 | −0.7875 | −0.5005 | −0.8595 | −0.7595 | −0.5095 |
| PTS | −0.564 | −0.084 | −0.171 | −0.247 | −0.363 | 0.388 | −0.643 | 0.218 | 0.143 | 0.042 |
| PTTG1 | −0.5245 | −0.3225 | −0.8315 | −0.1415 | −0.0005 | 0.5625 | −0.2945 | 0.0185 | −0.2675 | −0.7485 |
| PURB | −0.6185 | −0.7475 | 1.0725 | −0.1835 | 1.2515 | −0.7205 | 0.4275 | −0.1145 | 1.4105 | 0.0925 |
| PXMP2 | −0.3685 | −0.8385 | 0.4125 | −0.8555 | −0.3195 | −0.2955 | −0.8745 | −0.3705 | −0.0125 | −0.3765 |
| PYCARD | −0.133 | −0.444 | −0.234 | 0.698 | 0.393 | 1.236 | 1.463 | 0.764 | 0.539 | 0.701 |
| PYCR1 | 0.005 | −0.342 | 1.021 | −0.48 | −0.829 | −1.031 | −1.192 | −0.311 | 0.002 | −0.209 |
| QDPR | 0.2325 | 0.1885 | 1.1755 | −0.1495 | −0.1945 | −0.1385 | 0.5075 | 0.6375 | 0.9015 | 0.5015 |
| QPRT | −1.056 | −0.888 | −0.47 | −0.907 | −0.418 | 0.352 | 0.398 | 2.67 | 1.552 | 0.956 |
| QSCN9 | −1.2925 | −1.0395 | 0.1355 | −0.3985 | −1.3735 | −0.1265 | −0.2465 | 0.3325 | 0.1955 | −0.2415 |
| RAB11FIP | −0.502 | −0.704 | 0.227 | −0.558 | 0.029 | −0.114 | 0.019 | −0.257 | 0.142 | 0.063 |
| RAB11FIP | −0.584 | 0.155 | −0.043 | −3.05 | −0.814 | −0.031 | −0.287 | −0.181 | −0.274 | 0.159 |
| RAB13 | 0.011 | −0.041 | 1.447 | 0.412 | 0.258 | −0.002 | 0.317 | 0.889 | 0.445 | 0.835 |
| RAB27A | 0.06 | 0.307 | 0.101 | −0.567 | −0.251 | 0.081 | −0.338 | 0.207 | 0.419 | 0.695 |
| RAB30 | −0.6415 | −0.8345 | −0.2865 | −0.5765 | −0.3085 | −0.0685 | −0.8735 | −0.7345 | −0.7195 | −0.4535 |
| RAB34 | 0.591 | 0.397 | 0.873 | 0.392 | −1.003 | −0.32 | −0.859 | −0.882 | 0.797 | 0.048 |
| RAB35 | 0.088 | 0.588 | 0.086 | −0.001 | 0.075 | 0.51 | −0.085 | −0.08 | −0.179 | 0.019 |
| RAB39B | −0.6515 | −0.6275 | −0.2405 | −0.4225 | −0.4095 | −0.3085 | 0.2395 | −0.0165 | −0.2215 | 0.0245 |
| RAB3A | −1.097 | −0.176 | 0.141 | 0.671 | −0.679 | 0.696 | 0.313 | −0.411 | 1.078 | −0.849 |
| RAB3IP | −0.224 | −0.616 | 0.289 | 0.218 | 0.104 | 0.448 | 0.233 | −0.04 | 0.173 | 0.182 |
| RABEP1 | −1.037 | −1.308 | −0.969 | −0.701 | −1.113 | −1.017 | −0.607 | −1.332 | −1.132 | −1.194 |
| RABGAP1 | −0.0155 | −0.2875 | 0.3235 | 0.1875 | −0.0245 | 0.3505 | 0.3035 | 0.3945 | 0.1005 | −0.4235 |
| RACGAP1 | −1.0115 | −0.7305 | −1.0465 | −1.6155 | −1.0715 | −1.3095 | −0.6205 | −1.3105 | −1.2895 | −1.1225 |
| RAD17 | −0.193 | −0.167 | −0.646 | −0.888 | −1.2 | −0.059 | −0.35 | −0.439 | −0.478 | −0.536 |
| RAD51 | 0.14 | −0.12 | −0.155 | 0.063 | 0.082 | −0.06 | 0.394 | −0.302 | 0.043 | 0.089 |
| RAD51AP | −0.7525 | −0.1485 | −0.9445 | −1.4605 | −1.2885 | −1.0585 | −1.4455 | −1.1925 | −0.7655 | −0.9475 |
| RAD52 | −0.526 | −1.288 | −0.356 | −1.308 | −0.556 | −0.229 | 0.124 | −1.07 | −1.036 | −0.938 |
| RAD54L | −0.1525 | 0.0585 | −0.2515 | −0.7885 | −0.8305 | −0.0465 | −0.5765 | −0.7105 | −0.3825 | −0.5575 |
| RAMP | −0.6635 | 0.3335 | 1.5745 | 0.2345 | 0.0035 | −0.4215 | 0.8135 | 2.3085 | 1.2835 | −0.2465 |
| RANBP1 | −0.0505 | −0.6125 | 0.3865 | −0.2815 | −0.2625 | −0.2175 | −0.2825 | 0.0875 | −0.2785 | −0.4885 |
| RARRES1 | 0.387 | 0.723 | −0.005 | −0.787 | −0.485 | −1.233 | −0.009 | 1.705 | 0.172 | −0.063 |
| RBBP7 | −0.3225 | 0.0745 | −0.6075 | −0.0335 | −0.2195 | 0.1125 | −0.2565 | −0.0885 | −0.0095 | −0.0425 |
| RBBP8 | −0.5915 | −0.5685 | 0.1505 | 0.1305 | −0.7875 | 0.0365 | −0.2585 | 0.0025 | 0.1655 | −0.2555 |
| RBM8A | −0.586 | −0.511 | −0.378 | 1.219 | −0.502 | 0.756 | 0.705 | 0.197 | −0.314 | 0.848 |
| RBX1 | 0.583 | 0.606 | 0.046 | 0.421 | −0.147 | −0.031 | 0.128 | −0.066 | 0.352 | −0.002 |
| RDHE2 | −0.086 | −0.115 | 0.594 | −0.151 | −0.316 | 0.395 | 0.146 | 0.34 | 0.163 | 0.103 |
| REA | −0.006 | −0.681 | −0.186 | 0.42 | −0.105 | 0.063 | −0.403 | −0.432 | −0.211 | 0.09 |
| REC14 | −0.5345 | −0.0145 | 0.2985 | −0.6035 | −0.0355 | 0.3415 | −0.1635 | −0.2545 | 0.2625 | 0.7065 |
| REG1A | −0.029 | −0.222 | −0.279 | 0.384 | −0.401 | 0.307 | −0.386 | −0.32 | 0.171 | −0.306 |
| RELB | −0.758 | −1.595 | 0.659 | −0.247 | 0.177 | −0.45 | 0.059 | 0.004 | −0.382 | −0.401 |
| REPIN1 | −0.437 | −0.533 | 0.603 | 0.131 | 0.425 | −0.256 | 1.227 | 0.653 | 0.696 | 0.015 |
| REPS2 | −0.6355 | −0.2645 | −0.3445 | −0.3415 | −0.4115 | 0.0025 | −0.2335 | −0.3685 | 0.4135 | −0.7035 |
| RERG | −0.9476 | −0.7435 | 0.2715 | 0.4335 | −0.9325 | −0.4555 | 0.0735 | −0.1265 | 0.0545 | 0.0255 |
| RFC4 | −0.3285 | −0.6355 | 0.3545 | −0.9125 | −0.9215 | 0.0345 | −0.4095 | −0.0065 | −0.0355 | −0.5905 |
| RGS10 | −0.4475 | 0.1045 | 1.0205 | −0.5005 | −0.9695 | 0.6265 | −0.7775 | −0.1685 | −0.5525 | −1.1515 |
| RH8DL2 | −0.368 | −0.211 | 0.192 | −0.77 | −0.28 | −0.151 | −0.564 | −0.02 | 0.193 | 0.096 |
| RIBC2 | −0.299 | −1.433 | −0.363 | −0.6 | −0.765 | −0.412 | −0.442 | −0.825 | −0.195 | 0.054 |
| RIC-8 | −0.065 | 0.243 | 0.839 | −1.105 | 0.196 | −0.152 | 0.555 | 1.72 | 3.621 | −0.168 |
| RIT1 | −0.159 | 0.04 | −0.304 | −0.134 | 0.026 | −0.24 | −0.382 | −0.35 | 0.111 | 0.074 |
| RLN2 | −0.182 | −0.565 | 0.309 | −0.634 | −0.128 | 0.025 | −0.247 | 0.281 | 0.466 | −0.278 |
| RNF10 | −0.2915 | −0.5725 | 0.3485 | −0.1465 | −0.2055 | 0.1895 | −0.2245 | −0.1285 | 0.0745 | 0.0085 |
| RNF135 | 3.6295 | 3.3585 | 1.0505 | 0.3635 | −0.5565 | 0.9915 | 0.7355 | 2.0705 | 0.1735 | 1.0015 |
| RNF7 | −0.568 | −0.526 | 0.388 | −0.159 | 0.374 | −0.11 | −0.044 | −0.113 | −0.309 | −0.553 |
| ROPN1 | 0.5665 | −0.3085 | 1.2065 | −0.2525 | −1.4225 | 0.0415 | −0.2215 | 1.9335 | 1.4155 | 0.8945 |
| RPA3 | 0.8215 | 0.4755 | 0.3305 | 0.6235 | 0.5555 | 0.7945 | 0.1955 | 0.7065 | 0.1825 | 0.3665 |
| RPESP | 1.003 | 1.723 | 0.294 | 0.026 | 0.171 | 1.004 | 0.02 | 0.473 | 0.018 | 0.927 |
| RPL11 | 0.734 | 0.655 | 0.258 | 0.717 | 0.367 | 0.291 | 0.491 | 0.333 | 0.152 | 0.126 |
| RPL13 | 0.735 | 0.834 | 0.053 | 0.284 | 0.4 | 0.88 | 0.542 | 0.493 | 0.93 | 0.4 |
| RPL13A | 0.9545 | 0.9385 | 0.4065 | 0.2995 | 0.6045 | 0.0975 | 0.1815 | 0.0295 | 0.4805 | −0.1505 |
| RPL15 | 0.341 | 0.048 | 0.033 | −0.143 | 0.136 | 0.08 | −0.055 | 0.128 | 0.066 | −0.024 |
| RPL18A | 1.183 | 0.905 | 0.334 | 0.407 | 0.418 | 1.171 | 0.796 | 0.508 | 0.83 | 0.378 |
| RPL19 | 0.248 | 0.321 | 0.193 | −0.136 | 0.109 | 0.27 | 0.055 | 0.277 | 0.082 | 0.078 |
| RPL21 | 0.1225 | 0.2585 | 0.1815 | 0.1635 | 0.1885 | 0.0685 | −0.0365 | 0.2335 | 0.0405 | 0.1095 |
| RPL23A | 0.716 | 0.279 | 0.689 | 0.328 | 0.565 | 0.486 | 0.768 | 0.938 | 0.34 | 0.374 |
| RPL23A | 1.0465 | 1.0505 | 0.4425 | 0.4645 | 0.2045 | 0.1615 | 0.3955 | 0.3975 | 0.6355 | 0.1555 |
| RPL26L1 | 0.6835 | 1.3035 | −0.1825 | 0.4115 | 0.1845 | 0.1635 | 0.0465 | −0.1825 | 0.3785 | 0.3075 |
| RPL29 | −0.1485 | −0.0465 | 0.5535 | 0.2485 | 0.4185 | 0.1595 | 0.1245 | 0.2925 | 0.1435 | 0.0395 |
| RPL35 | 0.2035 | 0.5375 | 0.3075 | −0.4176 | 0.1075 | 0.8345 | −0.1975 | −0.0385 | 0.1345 | −0.1215 |
| RPL35AL | 0.6345 | 1.2195 | −1.1305 | −0.2575 | −0.6205 | 0.4155 | −0.4155 | −0.3695 | 0.4585 | 0.0205 |
| RPS16 | 0.0325 | 0.1085 | 0.3955 | 0.0025 | 0.4575 | 0.2325 | 0.2725 | 0.3595 | 0.1585 | 0.3315 |
| RPS4Y1 | 0.1155 | 0.5425 | 1.3355 | −0.0395 | 0.1915 | 0.3095 | −0.0035 | −0.7715 | −0.4255 | −0.0245 |
| RRAGA | −0.632 | −0.744 | −0.106 | −0.189 | −0.306 | −0.279 | 0.106 | −0.181 | 0.256 | 0.082 |
| RRAGD | −0.935 | −1.207 | −1.641 | −1.822 | −2.124 | −1.405 | −1.602 | −2.458 | −1.097 | −1.606 |
| RRM1 | 0.049 | −0.011 | 0.333 | 0.181 | 0 | 0.534 | −0.071 | −0.14 | −0.321 | 0.031 |
| RRM2 | 0.932 | 0.574 | 1.549 | 0.97 | 0.247 | 1 | 1.333 | 1.335 | 1.037 | 0.143 |
| RSAFD1 | −0.1205 | −0.8755 | 0.0255 | −0.1095 | −0.1385 | −0.2365 | −0.2235 | 0.0625 | 0.1595 | 0.2355 |
| RSNL2 | −0.0815 | −0.0655 | −0.0895 | −0.0725 | −0.3105 | −0.0145 | −0.8485 | 0.3905 | 0.1455 | −0.3865 |
| RSU1 | −1.102 | −0.915 | −0.47 | 0.66 | −0.083 | −0.725 | −0.282 | −0.681 | −0.101 | −0.304 |
| RTN4IP1 | −1.55 | −1.268 | −0.538 | −0.642 | 0.456 | −0.176 | −0.325 | −0.086 | −0.121 | −0.021 |
| S100A11 | −0.761 | −0.494 | −0.72 | −0.241 | 0.702 | −0.985 | 0.116 | 0.179 | 0.271 | 0.733 |
| S100A14 | −0.3095 | −0.6835 | 0.0155 | −1.6845 | −0.8885 | −0.2745 | −0.8045 | 0.1925 | 0.8885 | 0.2335 |
| S100A16 | −0.2725 | −0.4025 | −0.1555 | −0.4795 | −0.6155 | −0.1875 | −0.8205 | −0.5945 | −0.4085 | −0.1285 |
| S100A9 | 0.584 | 0.519 | 0.443 | 0.021 | 0.156 | 0.414 | 0.802 | 1.131 | 0.531 | 0.13 |
| SAE1 | −0.8755 | −0.8945 | −0.0005 | 0.4925 | 0.5475 | −0.4015 | 0.0285 | −0.3545 | −0.6345 | 0.1515 |
| SALL2 | −0.546 | −0.808 | 0.284 | 0.051 | −0.025 | −0.309 | 0 | −0.217 | −0.278 | −0.126 |
| SARA1 | −0.1945 | −0.5695 | −0.0345 | 0.3825 | −0.1955 | 0.0925 | 1.1055 | 0.1075 | 0.3925 | 0.2865 |
| SARA2 | −0.627 | −0.463 | 0.953 | 3.475 | −0.159 | 0.28 | −0.15 | −0.015 | −0.093 | 0.632 |
| SAS | 0.328 | 0.16 | −0.33 | 0.682 | −0.854 | 0.087 | 0.217 | −0.194 | −0.065 | 0.011 |
| SCIN | 0.0285 | −0.0645 | 0.6005 | 0.1945 | −0.0135 | −0.0855 | 0.7945 | 0.6625 | 0.5305 | 0.0585 |
| SCML1 | −0.219 | −0.377 | 0.138 | −0.855 | 0.127 | −0.783 | −0.459 | −0.068 | −0.389 | −0.363 |
| SCPEP1 | −1.1135 | −0.9705 | −0.8495 | −0.7416 | −0.5875 | −1.2175 | −1.5365 | −0.2505 | 0.4315 | 0.5135 |
| SCRIB | 0.564 | 0.533 | 0.449 | −0.058 | −0.038 | −0.359 | −0.277 | 0.781 | −0.682 | 0.422 |
| SDC1 | 0.127 | 0.233 | 0.179 | −0.111 | 0.013 | 0.222 | 0.034 | −0.131 | 0.002 | −0.002 |
| SDC3 | −0.8355 | −0.6125 | 0.5625 | 0.3415 | −0.0795 | −0.5835 | −0.7455 | −0.2945 | −0.5215 | −0.2795 |
| SDCCAG0 | −0.2615 | 0.3285 | −0.4145 | −0.5176 | −0.3785 | −0.1705 | −0.3555 | −0.0555 | −0.1225 | −0.1185 |
| SEC61G | −0.535 | 0.213 | 0.133 | 0.072 | 0.176 | 0 | 0.219 | 0.163 | −0.139 | −0.024 |
| SECP43 | −0.0225 | 0.8755 | −0.3835 | 0.6435 | 0.0065 | 1.9645 | 0.1575 | 0.7265 | 0.6815 | 0.7835 |
| SEDLP | 0.526 | −1.18 | 0.119 | 1.559 | 0.829 | 0.773 | 1.955 | 1.551 | 1.08 | 0.9 |
| SEMA3B | −0.4455 | 0.1785 | −0.0725 | −0.3835 | 0.2165 | 0.5065 | −0.0545 | −0.1745 | −0.1945 | 0.0565 |
| SEMA3C | −0.2255 | −0.8545 | 1.0585 | 0.6185 | 0.0045 | −0.1585 | 0.5525 | 1.0415 | 0.9505 | −0.0825 |
| SEPW1 | 0.867 | 0.916 | −1.088 | 0.148 | 0.175 | −1.063 | 1.35 | 2.269 | 0.188 | −0.709 |
| SERP1 | 0.7735 | 0.4575 | −0.7915 | −0.6825 | −1.3945 | 0.3165 | −0.4955 | 0.8645 | 0.5575 | −0.0105 |
| SERPINA [symbol] | 3.557 | 3.813 | 1.845 | 1.344 | 1.23 | 1.351 | 1.77 | 3.214 | 0.543 | 1.36 |
| SERPINB [symbol] | −0.053 | −0.194 | −0.559 | 0.505 | 0.089 | −0.757 | −0.874 | 0.593 | 0.004 | 0.448 |
| SFRP1 | 0.285 | 0.233 | 0.469 | 0.837 | 0.606 | 0.271 | −0.087 | −0.631 | 0.739 | 1.107 |
| SFRS6 | 0.9475 | 0.1195 | 0.2895 | −0.3995 | 0.7235 | 0.6595 | 1.0225 | 1.4655 | 0.5755 | 0.6405 |
| SGCD | −0.3745 | −1.1495 | 2.2055 | 0.8645 | −0.0995 | 0.7285 | 0.9315 | 1.5545 | 0.5505 | −0.0355 |
| SGCE | −0.436 | −0.448 | 0.072 | 0.018 | 0.351 | −0.348 | −0.273 | −0.176 | −0.439 | −0.193 |
| SH3BGRL | −0.07 | 0.21 | 0.744 | −0.553 | 0.281 | 0.741 | 0.722 | 0.809 | −0.256 | −0.358 |
| SHFM1 | −0.0245 | 0.3065 | −0.1305 | 0.3985 | 0.1045 | −0.5495 | −0.0835 | −0.3175 | −0.0935 | 0.4875 |
| SIAT7B | −0.176 | −0.074 | −0.318 | 0.615 | −0.946 | 0.151 | 0.455 | −1.057 | 0.405 | −0.149 |
| SIGLEC12 | 0.446 | 0.618 | 0.55 | −0.361 | 0.515 | −0.004 | −0.468 | 0.147 | 0.014 | −0.245 |
| SIT | 0.2405 | −0.2185 | 0.7315 | 0.4955 | 0.2575 | 0.5955 | 0.4475 | 0.6145 | 0.0265 | −0.4165 |
| SITPEC | 0.0675 | −0.0515 | 0.0455 | −0.0885 | 0.5035 | −0.4635 | −0.2345 | −0.1755 | −0.1125 | 0.0455 |
| SKP1A | 0.2045 | 1.2635 | 1.6545 | 1.0435 | 0.4305 | 0.2635 | −0.6935 | −0.1085 | −0.5785 | 0.5335 |
| SLC12A9 | −0.695 | −0.403 | 1.041 | −0.072 | 0.268 | −1.195 | 0.743 | 0.931 | 1.237 | 0.365 |
| SLC13A3 | −0.6485 | −0.4485 | −0.7065 | 0.1995 | −0.1895 | −0.4275 | 0.4235 | 0.4385 | 0.0275 | 0.1845 |
| SLC16A6 | 0.068 | −0.889 | 2.006 | 0.594 | −0.862 | −0.597 | 2.702 | 0.463 | 1.931 | 1.278 |
| SLC19A2 | −0.0155 | 0.5955 | −1.0145 | −0.6585 | −0.5805 | −0.5685 | 0.7095 | 0.0165 | 1.2505 | −0.5045 |
| SLC1A1 | −0.5305 | −0.3705 | −0.3815 | 0.1725 | 0.4215 | 0.1855 | −0.4695 | 0.9225 | −0.2485 | 0.3325 |
| SLC1A2 | 0.3245 | 0.3065 | 0.0245 | 0.5585 | 0.7145 | 0.0975 | 0.2555 | 0.2385 | −0.6545 | −0.4085 |
| SLC24A3 | −0.188 | −0.044 | −0.325 | 0.485 | −0.113 | 0.114 | 0.117 | 0.204 | −0.074 | −0.162 |
| SLC25A29 | −0.052 | −0.366 | 0.326 | −0.457 | −0.476 | −0.321 | −0.385 | −0.137 | −0.106 | −0.419 |
| SLC25A4 | −0.528 | 0.548 | 1.535 | 0.186 | −0.242 | 1.828 | 0.986 | 0.158 | 1.575 | 0.435 |
| SLC25A5 | 0.0775 | 0.5785 | 0.4975 | 0.0485 | −0.3505 | 0.2485 | −0.0035 | −0.1275 | 0.4935 | −0.4815 |
| SLC27A2 | 0.019 | −0.026 | −0.251 | −0.417 | 0.067 | 0.312 | −0.178 | 1.463 | 1.484 | 0.545 |
| SLC27A5 | −0.028 | 0.318 | −0.748 | −0.811 | −0.525 | −0.773 | −1.077 | −0.72 | −0.411 | −1.439 |
| SLC30A8 | −0.8325 | −1.5025 | 1.3995 | −1.7395 | −0.2605 | 1.1055 | 2.4335 | 2.6545 | 1.0985 | −0.3425 |
| SLC39A4 | −0.388 | −1.341 | 3.094 | 2.527 | 1.165 | 1.281 | 1.726 | 1.264 | 1.416 | 1.309 |
| SLC39A6 | 0.1225 | 0.6535 | 0.3585 | −0.2505 | −0.4025 | 0.0095 | −0.4755 | −0.1905 | 0.2855 | 0.1745 |
| SLC40A1 | 0.6015 | 0.2535 | −0.9935 | −0.9225 | −0.3285 | 0.0845 | −1.1395 | −0.2315 | 0.3495 | −0.8485 |
| SLC41A3 | 0.037 | 0.068 | 2.237 | −0.797 | −0.329 | 0.204 | 0.251 | −0.417 | 2.124 | 0.495 |
| SLC5A6 | −1.1055 | −0.5535 | −0.3355 | −0.2455 | −1.0185 | −1.2535 | −0.9605 | −0.0005 | −0.1185 | −0.2555 |
| SLC6A4 | −0.2465 | −0.0805 | 2.2965 | 1.5385 | −1.9125 | 0.2385 | 1.2785 | 2.3455 | 1.8875 | 1.7915 |
| SLC9A3R1 | −0.3485 | 0.4455 | 0.4405 | −0.7515 | −0.7275 | 2.2425 | −0.0755 | 0.9925 | 2.8875 | −0.0455 |
| SLITRK6 | −0.294 | −0.51 | −0.444 | −0.406 | −0.591 | 0.012 | −0.007 | 0.216 | 0.347 | −0.335 |
| SMA3 | 0.855 | 0.621 | 0.459 | −0.23 | −0.056 | 0.014 | −0.108 | 0.146 | 0.221 | 0.427 |
| SMAD2 | 0.164 | 0.847 | 0.088 | −0.15 | −0.282 | 0.455 | −0.275 | −0.041 | −0.062 | −0.252 |
| SMO | 0.183 | −0.249 | −0.044 | 0.169 | 0.037 | −0.073 | 0.106 | 0.332 | 0.047 | 0.02 |
| SMU1 | −0.037 | −0.245 | −0.022 | −0.24 | −0.179 | −0.219 | −0.269 | 0.019 | −0.267 | −0.264 |
| SNPH | −0.153 | 0.017 | −0.405 | −0.658 | −0.517 | 0.09 | −0.272 | −0.126 | −0.023 | −0.383 |
| SNRPB2 | −0.8355 | −0.8745 | −0.0235 | −0.6065 | −0.8125 | 0.2515 | −0.3795 | 0.0775 | −0.3615 | −0.8925 |
| SNRPD1 | −0.524 | −0.951 | −0.351 | 0.28 | −0.035 | −0.573 | 0.181 | −0.178 | −0.368 | −0.415 |
| SNRPD3 | 0.0365 | −0.6935 | 0.4625 | −0.2475 | 0.3265 | −0.8845 | 0.2705 | 0.6885 | 0.4885 | 0.5855 |
| SNRPG | −0.4405 | 1.6745 | 0.1485 | −0.8895 | 0.0015 | 0.2295 | −1.1595 | −0.0315 | 0.6415 | −0.1285 |
| SNX7 | 0.2785 | −0.4055 | 0.1816 | −0.0515 | −0.0495 | 0.1055 | −0.0285 | −0.3165 | −0.2395 | −0.0045 |
| SO02 | −0.5635 | −0.3275 | 0.5475 | 0.9525 | −0.2515 | −1.3135 | 0.0415 | −0.1575 | −0.7455 | −0.5125 |
| SORCS1 | 0.428 | 0.827 | 0.111 | 0.328 | 0.168 | −0.196 | 0.129 | −0.01 | 0.946 | 0.295 |
| SOX4 | 0.5055 | 0.0465 | 2.0785 | −0.2245 | −0.2835 | 0.0455 | 0.4005 | 0.7965 | 0.2725 | −0.1595 |
| SOX9 | −1.496 | −0.234 | −1.318 | −1.552 | −1.095 | −1.225 | −0.878 | −1.534 | −1.373 | −1.624 |
| SPAG18 | 0.1055 | −0.5685 | 0.9485 | −0.0095 | 0.4445 | 0.9165 | −0.0265 | 0.8325 | 0.4995 | −0.0075 |
| SPAG5 | −0.3565 | −0.4715 | 0.0805 | 0.3305 | 0.1395 | −0.0025 | −0.1995 | 0.0895 | −0.3415 | −0.4845 |
| SPC18 | 0.184 | −0.084 | 0.288 | −0.231 | −0.777 | 0.335 | −0.289 | 1.032 | 0.891 | 0.289 |
| SPFH2 | −0.579 | −0.131 | −0.55 | −0.388 | −0.349 | −0.456 | 0.215 | 0.011 | 0.091 | −0.04 |
| SPG3A | −1.572 | −2.18 | 0.215 | −1.745 | −1.585 | −1.005 | −1.075 | −0.555 | −0.2 | −0.825 |
| SPPL3 | −1.138 | −0.842 | −0.233 | 0.737 | −0.308 | 0.732 | −0.452 | 0.178 | 0.386 | 0.337 |
| SQLE | −0.82 | −0.553 | −1.333 | −0.537 | −1.292 | 0.988 | −0.429 | −0.103 | 0.703 | 0.643 |
| SQRDL | 0.0405 | −0.2255 | 0.2475 | 0.0545 | 0.8335 | 0.4345 | 0.9495 | 1.1075 | 0.6495 | 0.0025 |
| SREBF1 | −0.4005 | −0.8435 | −0.3745 | −0.9915 | −0.8515 | −0.5745 | −0.8825 | −0.4875 | −0.2355 | −0.4575 |
| SRI | 0.1035 | 0.1505 | 0.7045 | 0.1925 | 0.1545 | 0.5085 | 0.7085 | −0.1255 | 0.0935 | 0.3325 |
| SRPK1 | −0.0885 | 0.0905 | 0.2205 | 0.0015 | −0.8745 | 0.3635 | −0.1205 | 0.0415 | −0.1695 | 0.1275 |
| SSFA2 | −0.4305 | 0.4505 | 0.2805 | 0.4025 | 0.8925 | 0.0005 | 0.3685 | 0.4905 | 0.2135 | 0.0725 |
| SSR4 | 0.0395 | −0.1075 | 0.5745 | 0.6775 | 0.2385 | −9.2135 | 0.2615 | 0.2205 | −0.0755 | −0.5545 |
| STARD3NI | −1.0795 | −1.3555 | −0.7185 | −0.4195 | −1.0365 | 0.7425 | −0.2475 | 0.1875 | −0.8215 | −0.4705 |
| STARD7 | 0.6865 | 0.5135 | 0.1355 | −1.2315 | 2.1185 | −1.0855 | 1.4175 | 1.8885 | 1.6125 | 0.4145 |
| STAT1 | −0.0965 | 0.0405 | −0.2765 | −0.5195 | −0.1425 | −0.0385 | −0.0715 | 0.0795 | 0.0035 | −0.0595 |
| STC2 | −0.3375 | −0.2345 | −0.8435 | −0.9115 | −0.7425 | −0.5975 | −0.7795 | −1.1915 | −0.3405 | −0.8115 |
| STK24 | −0.7855 | −1.5295 | −0.9185 | −1.1185 | −1.2465 | −1.2585 | −1.2435 | −1.1265 | −0.9705 | −1.2995 |
| STK6 | −0.369 | −0.453 | −0.797 | −0.493 | −0.725 | −0.376 | −0.724 | −0.829 | −0.478 | 0.298 |
| STMN1 | −0.5015 | −0.3235 | −0.0465 | −0.8085 | −1.2925 | −0.8105 | −0.6255 | −0.0625 | −0.0235 | 0.1055 |
| STX1A | −0.0045 | 0.6885 | 0.0045 | 0.2135 | −0.2705 | −0.0685 | −0.1225 | 0.0055 | 0.1405 | −0.4875 |
| STX3A | −0.5555 | −0.2825 | −0.1015 | −0.2495 | −0.2275 | −0.3095 | −0.2065 | −0.3625 | −0.8325 | −0.4875 |
| STYX | −0.8585 | −0.3055 | 0.6815 | −0.0975 | 0.1295 | 0.7895 | 0.2095 | 0.5765 | 0.5545 | −0.0295 |
| SUGT1 | −0.118 | 0.027 | −0.276 | −0.806 | −0.549 | 0.123 | −0.366 | −0.439 | −0.099 | −0.143 |
| SUMF1 | 0.2945 | 0.2895 | −0.2245 | −0.2735 | −0.4925 | −0.0855 | −0.6675 | 0.0845 | 0.1745 | −0.5315 |
| SUMO2 | −0.5935 | 0.6895 | 0.0585 | −0.8655 | −0.9085 | 0.0855 | −0.5345 | −0.1575 | −0.4245 | −0.5355 |
| SUV39H2 | −1.1595 | −1.8685 | −1.8445 | 0.1905 | 0.1445 | 0.2385 | 1.0725 | −0.5815 | −0.8525 | −1.1995 |
| SYAP1 | 0.4615 | 0.9085 | −0.5425 | −0.2035 | −0.7085 | −0.4245 | −0.1115 | 0.2505 | −0.4475 | −0.0485 |
| SYCP2 | −0.656 | −0.802 | 0.035 | −0.894 | 1.054 | 0.156 | 0.401 | 1.457 | 0.11 | 0.68 |
| SYNGR1 | −0.3815 | 0.4905 | 0.0325 | −0.5875 | −0.4845 | −0.3665 | 0.1285 | 0.3585 | 0.5535 | −0.0045 |
| SYTL2 | −0.4395 | −0.6005 | −0.2965 | −0.3375 | 0.0665 | −0.4455 | −0.3585 | −0.1095 | −0.4125 | −0.1235 |
| TAF18 | −0.0655 | −0.5775 | −0.4785 | 0.2625 | 0.5005 | −0.0035 | 0.6905 | −0.3185 | 0.6605 | 0.2885 |
| TAF8 | −0.439 | −0.632 | −0.522 | −0.421 | −0.507 | 0.144 | −0.544 | −0.37 | −0.102 | −0.188 |
| TARBP1 | 0.275 | −0.045 | 0.785 | −0.275 | −0.043 | 0.297 | −0.099 | 0.422 | 0.175 | −0.48 |
| TARS | 0.2715 | −0.0565 | 0.4755 | −0.6395 | −0.2705 | 0.7735 | 0.3485 | 0.0605 | −0.0635 | −0.0215 |
| TATDN1 | 0.2405 | 0.0045 | 0.0635 | 0.1816 | −0.4365 | −0.3905 | −0.4325 | 0.2655 | 0.3955 | −0.5365 |
| TBC1D12 | 0.1616 | −0.2165 | −0.3535 | −1.0955 | 0.2685 | −0.2515 | 0.3255 | −0.5645 | −0.0825 | −0.3615 |
| TBC1D14 | −0.133 | −0.507 | 0.243 | 0.453 | 0.161 | 0.388 | −0.104 | 0.07 | −0.273 | −0.574 |
| TBC1D3 | −0.1665 | 0.0195 | 1.5555 | −0.1845 | −1.1045 | −0.7275 | 0.0595 | −0.6705 | −1.4525 | −0.3975 |
| TBCA | −0.105 | −0.355 | 1.528 | 0.585 | 1.221 | −0.069 | 1.334 | 0.946 | 0.913 | 0.361 |
| TBX1 | −0.3295 | −0.1895 | −0.0635 | −0.5115 | −0.8105 | 0.0465 | −0.5335 | −0.5375 | −0.5065 | −0.2295 |
| TBX3 | 0.16 | 0.055 | −0.083 | 0.182 | 0.107 | 0.465 | 1.814 | 0.511 | 0.242 | 0.405 |
| TCAP | −0.063 | 0.098 | −0.285 | −0.016 | 0.4 | 0.371 | 1.188 | 0.999 | 0.18 | 0.885 |
| TCEAL1 | 0.1105 | −0.3585 | 0.2565 | −0.1555 | 0.5155 | 0.1965 | 0.8055 | 0.2805 | 0.1225 | 0.7595 |
| TCEAL3 | −1.025 | −1.188 | 0.147 | −1.596 | 0.041 | −0.624 | −0.554 | −1.948 | −0.498 | 0.114 |
| TCEAL4 | −0.723 | −0.61 | −0.328 | 0.1 | −0.403 | −0.64 | −0.194 | −0.287 | −0.152 | −0.213 |
| TCF15 | 1.035 | 0.163 | 0.49 | 0.873 | 0.464 | 0.268 | 0.138 | 0.078 | 0.49 | 0.427 |
| TCF3 | −0.338 | −0.61 | −0.169 | −0.387 | −0.427 | 0.399 | −0.067 | −0.327 | −0.213 | −0.612 |
| TCF7L2 | 0.0675 | −0.1075 | 0.2435 | 0.8766 | −0.3385 | −0.3385 | 0.3645 | 0.2895 | −0.1655 | −0.2575 |
| TDRD7 | −1.0075 | −1.5495 | −0.5075 | 2.3595 | 1.9235 | 0.2335 | 1.2575 | 1.3515 | 0.9055 | 0.8895 |
| TESK2 | 0.222 | 0.891 | 3.138 | −0.322 | −1.559 | −0.941 | 1.239 | 0.873 | −0.586 | −1.423 |
| TFAP2B | 0.527 | −0.161 | 0.258 | 0.672 | −1.038 | −1.264 | 0.493 | 0.491 | −0.358 | −0.291 |
| TFCP2L1 | −0.1445 | 0.3745 | 0.9265 | −0.4945 | 2.6805 | 0.1125 | 1.3565 | 2.7275 | −0.0915 | 0.9815 |
| TFCP2L2 | −0.4685 | −0.4855 | 1.9775 | 2.0585 | −0.1705 | 0.0995 | 0.8535 | 2.5525 | −1.7155 | 0.5375 |
| TFF1 | −0.5505 | −1.0155 | 0.7285 | −0.5995 | −0.0435 | 0.0825 | 0.4005 | −0.0655 | −0.0215 | −0.3265 |
| TFF3 | 0.947 | 1.062 | 2.484 | 1.756 | 2.18 | 1.314 | 1.459 | 1.331 | 0.143 | 0.768 |
| TFRC | 0.2835 | 0.5575 | 0.0655 | 0.3005 | −0.1315 | 0.1785 | 0.3105 | 0.4155 | 0.3815 | 0.3305 |
| TGFBR3 | −0.936 | 0.005 | −0.049 | 0.885 | 0.468 | 0.115 | 0.377 | 0.087 | −0.715 | −0.759 |
| THY28 | 1.0855 | 1.3695 | 0.7795 | 0.8465 | 0.7125 | 0.8285 | 1.2255 | 1.0165 | 0.3945 | 0.3945 |
| TIF1 | −0.157 | −0.877 | 0.128 | −0.075 | −0.132 | 1.064 | 0.2 | 0.934 | 0.593 | −0.425 |
| TIGA1 | −1.076 | −0.987 | −0.382 | −1.298 | −1.069 | −1.238 | −0.599 | −0.58 | −0.416 | −0.983 |
| TIM14 | 0.1895 | 0.4575 | 0.8555 | 0.2055 | 0.0855 | 0.2165 | −0.0575 | 0.2525 | 0.0855 | −0.0125 |
| TIMELES [symbol] | 0.8755 | −0.0545 | 0.5105 | 0.5825 | 0.5035 | 0.6985 | 0.9045 | 1.5485 | 0.2755 | 0.1865 |
| TIMM9 | 0.352 | 1.15 | 1.159 | 0.384 | 0.624 | 1.103 | −0.214 | 0.249 | 0.147 | 1.035 |
| TINP1 | −1.6825 | −1.6325 | −0.4925 | −1.4235 | −0.9475 | −1.1015 | −1.0195 | −1.0195 | −0.5725 | −1.2575 |
| [symbol] IP1208 | 0.707 | 0.835 | 0.283 | 0.452 | 0.748 | 0.798 | −0.251 | 0.448 | −0.05 | −0.418 |
| TK1 | −0.88 | −0.897 | 0.632 | 0.724 | 0.528 | −0.336 | 1.025 | 0.19 | 0.353 | −0.531 |
| TLE2 | 0.011 | −0.489 | 2.103 | −0.004 | 0.085 | 0.496 | 0.667 | −0.035 | −0.472 | −0.048 |
| TLR5 | −0.784 | −1.357 | 1.485 | −0.446 | 0.166 | 1.039 | 0.725 | 0.802 | −0.363 | −0.342 |
| TM4SF12 | −0.841 | −0.595 | 0.105 | −0.212 | −0.083 | −0.573 | 0.024 | −0.059 | 0.25 | −0.323 |
| TM4SF13 | −0.83 | −1.802 | 0.1 | 1.854 | 0.15 | −0.753 | 0.077 | 0.905 | 0.964 | −0.031 |
| TMEM14A | −0.0945 | −0.1925 | 0.1976 | 0.0555 | −0.1325 | −0.3195 | −0.0825 | 0.1035 | −0.1505 | −0.4005 |
| TMEM25 | 0.7745 | 0.8185 | 0.1235 | −0.0295 | −0.5585 | 0.9935 | −0.0245 | 0.1865 | −0.8585 | 0.3835 |
| TMEM4 | −0.592 | −0.797 | 0.113 | −0.025 | −0.49 | −0.532 | −0.733 | −0.332 | −0.315 | −0.028 |
| TMPRSS5 | 0.67 | 0.05 | −1.16 | −1.528 | −1.045 | −0.483 | −0.88 | −0.695 | −0.24 | −0.488 |
| TMSB10 | 1.221 | 0.649 | 1.304 | 0.845 | −2.392 | 1.682 | 0.409 | 0.148 | −0.275 | 1.099 |
| TMSNB | 0.5285 | 1.1535 | 0.1515 | −0.1835 | −0.3835 | 0.4135 | −0.1335 | 0.6325 | −0.2435 | −0.4125 |
| TNFRSF1 [symbol] | 0.566 | 0.038 | 0.065 | 0.264 | 0.106 | 0.072 | 0.308 | 1.13 | −0.36 | 0.033 |
| TNFRSF2 [symbol] | −0.6855 | −0.7255 | −0.1535 | −0.5135 | −0.8735 | −0.3915 | −0.6725 | −0.9305 | −0.0435 | −0.0065 |
| TNFRSF2 [symbol] | 0.1275 | −0.1095 | −0.3035 | 1.4625 | 0.2045 | 0.4725 | −1.1275 | 1.2505 | −0.6315 | 1.8085 |
| TNFSF4 | −0.1145 | −0.1315 | 0.1155 | −0.0195 | 0.2275 | 0.0795 | 0.1155 | 0.2815 | 0.2585 | 0.0995 |
| TNFSF7 | 0.2025 | 0.7476 | −0.2045 | 0.2235 | 0.4205 | 0.9135 | −0.0635 | 0.7235 | −0.5405 | 0.2785 |
| TNIK | 2.1475 | 1.9895 | −0.7065 | 0.9235 | −0.8305 | 0.2985 | 1.0795 | 2.8905 | 0.7585 | 0.1045 |
| TNNC2 | 0.855 | 1.603 | 2.015 | −1.354 | 1.408 | 1.103 | −0.772 | −0.914 | 1.971 | −0.171 |
| TNNI2 | −0.578 | 0.885 | −0.089 | −0.066 | 0.302 | 0.374 | 0.001 | 9.247 | 0.193 | −0.068 |
| TNNI3 | −0.3095 | −0.8035 | 0.4475 | −0.4026 | 0.0065 | −0.4255 | 0.4165 | 0.5445 | 0.2025 | −0.6555 |
| TOB1 | 0.2935 | 0.8225 | 0.6505 | 0.5155 | 0.8215 | 0.7265 | 0.0535 | 0.5255 | −0.1175 | 0.1515 |
| TOM1L1 | 0.1305 | 0.3405 | 0.8755 | −0.9475 | −0.6835 | −0.0065 | −0.4435 | −0.4445 | −0.4025 | −0.5145 |
| TOMM7 | −0.9785 | −0.4925 | −0.6185 | −1.2165 | −0.7075 | −0.9855 | −0.5345 | −0.6575 | −0.7035 | −1.1205 |
| TOP1MT | 0.217 | −0.038 | −0.517 | 0.858 | 0.583 | 0.13 | 0.491 | 0.303 | 0.505 | 0.167 |
| TOP2A | −0.5715 | −0.0255 | 0.1975 | −0.2755 | 0.0735 | 0.0385 | 0.0585 | −0.7455 | −0.0445 | −0.8345 |
| TOP2B | −1.195 | −1.089 | −1.169 | −1.306 | −0.886 | −1.292 | −0.825 | −0.75 | −0.85 | −2.009 |
| TOPBP1 | −0.9325 | −0.6715 | 0.7365 | −0.2145 | 0.0935 | −0.1885 | 0.3485 | −0.0365 | −0.0665 | −0.0705 |
| TOPK | −0.3915 | −0.4745 | −0.0235 | 0.2635 | −0.6225 | −0.1925 | −0.1825 | 0.1755 | 0.5595 | 0.4545 |
| TP53AP1 | −1.1045 | −1.3145 | 0.7655 | 1.4735 | 0.2135 | 1.0065 | 1.1075 | 0.1665 | 0.0325 | −0.3075 |
| TP53BP2 | 0.8905 | 0.9885 | 0.6055 | 1.5365 | 0.2255 | −0.8205 | 0.6975 | 0.4255 | −0.1565 | −0.5635 |
| TP53INP1 | 0.1975 | −0.0495 | −0.5585 | −0.5605 | −0.8505 | −0.0055 | −0.4655 | −0.1255 | −0.2385 | −0.6175 |
| TPD52L1 | −0.583 | −1.558 | −0.141 | 0.587 | −0.658 | 0.549 | 0.826 | −0.016 | 0 | 0.208 |
| TPRT | 0.0865 | 0.5065 | −0.8655 | −0.4345 | 0.2885 | −0.2855 | −0.1845 | −0.2785 | −0.2076 | 0.3475 |
| TRA@ | 0.183 | −0.244 | 0.108 | 0.394 | 0.278 | 0.14 | −0.3 | −0.003 | −0.18 | 0.046 |
| TRAF4 | −0.107 | −0.103 | −0.102 | 0.052 | 0.344 | −0.465 | 0.092 | 0.13 | 0.272 | −0.126 |
| TRAPPC4 | −0.256 | −0.375 | 1.595 | −0.15 | 0.198 | −0.017 | −0.217 | 0.57 | 0.42 | −0.382 |
| TRH | 1.0855 | 1.4255 | 0.5045 | 0.0755 | 0.0325 | 0.6895 | 0.0895 | 0.1915 | −0.5365 | −0.0485 |
| TRIM17 | −0.0845 | −0.0065 | −0.6365 | 0.3655 | −0.1795 | −0.6645 | 0.2615 | 0.2765 | 0.2105 | 0.2185 |
| TRIM2 | −0.1795 | −0.5235 | 0.3155 | 0.1085 | 0.0205 | 0.0045 | 0.2515 | 0.3225 | −0.1085 | −0.3235 |
| TRIM6 | −0.951 | −1.533 | −0.043 | 0.861 | 0 | −1.065 | 0.66 | 0.437 | 0.58 | −1.073 |
| TRIP3 | −0.4795 | −0.5275 | 0.2295 | 0.3205 | 0.6115 | −0.1935 | 0.4005 | 0.1585 | −0.0345 | −0.0955 |
| TRPS1 | −0.2765 | −0.6225 | 0.1185 | 0.6655 | 0.6345 | 0.4535 | 0.7325 | 0.6925 | 0.2185 | 0.1055 |
| TSG101 | −0.2775 | −0.8805 | 0.2965 | 1.7335 | 0.9545 | 0.1705 | 0.3355 | 1.9035 | 0.6325 | 0.5195 |
| TTC10 | −0.6455 | −0.1445 | −0.6335 | −1.3355 | −0.5905 | −0.4505 | −1.0905 | −1.0275 | −1.1355 | −1.3675 |
| TTC12 | 0.452 | 0.49 | −0.259 | −0.543 | −0.075 | 0.202 | −0.438 | −0.58 | −0.011 | −0.073 |
| TTK | 2.1526 | 2.4505 | 0.2245 | 0.3435 | −0.9035 | 0.2555 | −0.0465 | 0.1585 | −0.5195 | 0.0675 |
| TTLL4 | 0.007 | −0.354 | −0.581 | −0.872 | −0.802 | −0.853 | −0.483 | −0.705 | −0.345 | 0.29 |
| TTYH1 | −0.0155 | 0.7835 | −0.0285 | 0.4715 | 0.2015 | 0.2555 | −0.0245 | −0.3285 | −0.4205 | −0.1905 |
| TUBB3 | −0.5605 | −0.4735 | 0.0765 | 0.1295 | −0.3175 | −0.4045 | −0.1115 | 0.0725 | −0.4455 | −0.7415 |
| TULP3 | −0.982 | −1.233 | −1.276 | −1.595 | −1.385 | −1.035 | −0.945 | −0.165 | −0.825 | −1.105 |
| TXN | −0.409 | 0.044 | −0.136 | −0.49 | −0.208 | −0.27 | −0.336 | −0.273 | −0.138 | 0.055 |
| TYMS | 0.4675 | 0.1105 | −0.2855 | −0.7285 | −0.5335 | 0.0776 | 0.3305 | 0.5825 | 0.8645 | 0.6065 |
| U5-116KD | −0.7505 | −0.7095 | −0.1645 | 0.0755 | −0.5895 | −0.1325 | −0.1545 | −0.1345 | −0.3375 | −0.5335 |
| UBE2E3 | −0.326 | −0.398 | −0.004 | −0.588 | 0.244 | 0.243 | −0.511 | −0.377 | −0.014 | −0.604 |
| UBL5 | −0.5445 | −0.0035 | 0.9475 | −0.6545 | −0.1585 | −0.3275 | −0.9615 | −0.9825 | 0.0035 | 0.6505 |
| UBN1 | −0.193 | −0.316 | −0.176 | 0.369 | 0.194 | −0.019 | 0.404 | 0.919 | 0.322 | 0.079 |
| UCHL1 | −1.61 | −0.372 | 1.56 | −0.164 | 1.983 | 0.89 | 0.571 | 0.463 | 0.843 | −0.243 |
| UCHL3 | −0.811 | 0.025 | 0.182 | −0.633 | −1.397 | −0.07 | −0.831 | −0.608 | −0.385 | −0.628 |
| UGCG | 0.2795 | 0.9725 | −0.8105 | −0.8375 | −1.0255 | −0.9005 | 3.4365 | −0.8895 | −0.8825 | 1.0885 |
| UGCGL1 | 0.0645 | 0.1435 | −0.8776 | −0.1965 | −0.2455 | −0.0985 | −0.3725 | 0.1385 | −0.2655 | 0.1175 |
| UGT2B7 | −0.8015 | −0.2575 | −0.0805 | −0.4545 | −0.9485 | −0.3976 | −0.3595 | −0.3515 | −0.4345 | −0.5985 |
| UNC13D | 0.143 | 0.377 | −0.025 | −0.187 | −0.16 | 0.779 | 0.465 | 1.066 | 0.862 | 0.596 |
| VAMP8 | −0.027 | 0.001 | 0.533 | −0.087 | −0.465 | 0.016 | 0.044 | −0.551 | −0.24 | 0.099 |
| VAPA | −1.2885 | −1.5215 | −0.9085 | 0.8975 | −1.0885 | 0.0345 | 0.2045 | 1.2175 | 1.6995 | −0.0085 |
| VAV1 | 1.088 | 2.746 | −0.714 | 0.827 | −0.533 | 0.457 | 0.114 | −0.164 | −0.109 | −0.351 |
| VAV3 | 0.026 | 0.237 | −0.341 | −0.244 | −0.174 | 0.136 | −0.158 | −0.187 | 0.009 | −0.099 |
| VCX3 | −0.362 | −0.517 | 0.152 | 0.257 | 0.53 | 0.154 | 3.396 | 0.547 | 1.145 | −0.099 |
| VDAC2 | −0.1325 | 0.1595 | 0.2265 | −0.0915 | −0.5115 | −0.3215 | −0.5115 | 0.1585 | −0.3685 | −0.0515 |
| VEST1 | 0.475 | 1.047 | −0.555 | 0.124 | 0.01 | 0.032 | −0.25 | −0.442 | 0.148 | 0.302 |
| VGLL1 | 1.1225 | 0.9785 | 1.0895 | 1.0845 | 0.7255 | −0.1765 | 0.6175 | 0.9835 | 0.0035 | 0.0055 |
| VMD2L2 | −0.441 | −0.207 | 0.111 | −0.352 | −0.282 | 0.319 | −0.029 | −0.072 | 0.448 | 0.293 |
| VSX1 | −0.539 | −0.968 | −0.552 | 0.424 | −0.066 | −0.43 | 0.09 | −0.421 | −0.035 | −0.009 |
| WARP | 0.55 | 0.803 | 0.103 | −0.328 | −0.78 | 0.023 | −0.043 | 0.054 | 0.368 | 0.014 |
| WDR26 | −0.2785 | −0.6165 | −0.3455 | 0.1895 | 0.2855 | −0.2995 | 0.1095 | −0.1045 | 0.0125 | −0.4605 |
| WDR6 | −0.36 | 0.235 | 0.232 | −0.898 | 0.118 | −0.274 | 1.072 | 0.183 | −0.6 | −0.264 |
| WHSC1L1 | 0.577 | 0.397 | 0.315 | −0.169 | −0.339 | −0.209 | 0.041 | 0.301 | −0.363 | −0.119 |
| WNT3 | −1.2685 | −1.1595 | −0.6885 | −0.3575 | −0.4145 | −0.4425 | −0.4115 | −0.4035 | 0.0225 | −0.0965 |
| WNT6 | −1.1115 | −1.1815 | 1.2155 | 0.0845 | 0.2855 | 0.3605 | 0.6225 | 0.2405 | −0.2045 | −0.6705 |
| WSB2 | −0.0505 | 0.3845 | 0.0435 | −0.0725 | −0.1825 | −0.3235 | −0.3105 | −0.0405 | −0.1315 | 0.2375 |
| WWP1 | −1.0805 | −1.0845 | 0.3205 | 0.4315 | −0.8175 | 0.0985 | 0.8525 | 1.0335 | 0.2315 | −0.0745 |
| WWP2 | −0.246 | 0.108 | −0.076 | −0.937 | −0.31 | −0.024 | −0.69 | 0.007 | −0.306 | −0.161 |
| XBP1 | −0.164 | −0.632 | 0.064 | −0.824 | −0.494 | −0.139 | −0.501 | −0.95 | −0.443 | −0.942 |
| XYLT2 | −0.4275 | −0.4305 | −0.2875 | −0.0395 | −0.2795 | −0.5765 | −0.6295 | −0.5045 | −0.2515 | 0.0895 |
| YBX2 | −0.236 | −1.196 | 0.502 | −0.048 | −0.117 | 0.533 | 0.588 | 0.51 | 0.436 | 0.257 |
| YIF1 | −0.5475 | −0.5225 | −0.1105 | 1.0805 | 1.0545 | 0.9765 | 0.7555 | 0.6425 | 0.1305 | −0.0775 |
| ZA20D3 | 0.0005 | 0.3615 | 0.0175 | 1.1975 | 1.0425 | −0.1285 | 0.4625 | −3.0815 | 0.2485 | 0.3355 |
| ZAP128 | 0.0555 | 0.3495 | 0.5075 | 0.4945 | 1.1955 | 0.4435 | 0.8965 | 0.6735 | 0.6965 | 0.5395 |
| ZBED3 | −0.304 | −0.217 | 0.188 | −0.236 | −0.556 | −0.577 | −0.414 | −0.821 | −0.839 | −0.414 |
| ZBTB4 | −0.243 | −0.106 | −0.099 | 1.182 | 0.331 | −0.06 | 0.09 | 0.035 | −0.314 | 0.041 |
| ZCWCC1 | −0.1305 | −0.5795 | 0.3285 | −0.3025 | −0.9725 | −0.6545 | −0.3355 | −0.4445 | −0.3935 | −0.3365 |
| ZCWCC2 | −0.058 | −0.688 | 0.681 | 0.838 | 0.858 | 0.038 | −0.403 | 0.672 | −0.089 | −0.474 |
| ZDHHC13 | 0.6105 | −0.2955 | 0.5095 | 1.5925 | 1.2795 | 0.7635 | 0.9375 | 1.0345 | 1.0245 | 1.5385 |
| ZDHHC14 | 0.1125 | −0.0165 | 0.2075 | 0.6865 | 0.7595 | 0.5145 | 0.4685 | 0.6765 | 0.2945 | 0.1525 |
| ZFHX4 | 0.6335 | 0.0995 | −0.1865 | 0.4435 | −0.3125 | −0.1085 | 0.2695 | −0.1255 | −0.0865 | −0.2145 |
| ZFYVE21 | −0.851 | 0.983 | 0.71 | −0.434 | −0.746 | −0.648 | −0.425 | 0.272 | −0.431 | −0.484 |
| ZNF133 | −0.129 | −0.271 | −0.27 | −0.434 | −0.341 | 0.51 | 0.494 | 0.323 | 0.475 | −0.207 |
| ZNF165 | 0.1365 | −0.1125 | −0.0045 | 0.0205 | −0.4045 | 0.0775 | −0.1055 | −0.4005 | −0.3025 | −0.5055 |
| ZNF24 | −0.347 | −0.24 | 0.217 | 0.15 | 0 | −0.065 | 0.558 | 0.236 | 0.47 | −0.114 |
| ZNF318 | 0.1815 | −0.2095 | −0.2775 | 1.1055 | 0.1335 | −0.2415 | 0.1395 | −0.1145 | −0.2785 | −0.0955 |
| ZNF54 | 1.0645 | 1.7435 | 0.1785 | 0.7095 | −0.2455 | −0.1625 | −0.1935 | 0.0705 | 0.0755 | 0.4545 |
| ZNF432 | −0.0795 | −0.6465 | −0.0335 | −0.1055 | −0.4245 | −0.4095 | −0.1645 | −0.0725 | −0.0805 | 0.1135 |
| ZNF482 | −1.207 | −0.675 | 0.596 | 0.399 | 0.052 | 0.16 | −0.088 | 0.546 | −0.026 | −0.566 |
| ZNF505 | 0.012 | −0.096 | −0.185 | 0.937 | 1.331 | 0.367 | 0.726 | 0.265 | 0.535 | 0.773 |
| ZNF552 | −0.9415 | −0.2555 | 0.8615 | 0.2875 | 0.2845 | −0.1995 | 0.2865 | 0.1885 | −0.5535 | −0.6715 |
| ZNF577 | −0.3905 | −0.2125 | 0.1885 | −0.5155 | −0.2955 | −0.2995 | −0.6285 | −0.4845 | 0.0115 | −0.3215 |
| ZNF587 | 0.0285 | −0.4825 | 0.1555 | 0.0595 | 0.0925 | 0.0455 | −0.0615 | 0.5865 | −0.1735 | −0.5315 |
| ZNF598 | −0.2435 | −0.8805 | −0.1325 | 1.0655 | −0.2155 | −0.0965 | 0.0535 | 0.0875 | −0.4255 | −0.3565 |
| ZNF599 | 0.312 | 0.079 | −0.312 | 1.436 | 0.392 | −0.245 | −0.248 | 0.214 | 0 | 0.297 |
| ZNF606 | −0.15 | −0.523 | −0.019 | −0.43 | −0.755 | −0.514 | 0.085 | 0.301 | 0.442 | −0.296 |
| ZNF614 | −0.128 | −0.288 | 0.38 | −0.26 | −0.524 | 0.215 | −0.021 | −0.085 | −0.135 | −0.309 |
| ZNF639 | | | | | | | | | | |
| ZNF7 | | | | | | | | | | |
| | Sample | | | | | | | | | |
| | BR96-001 [symbol] | UB-91 | FB284 | UB-22 | UB-95 | UB-85 | UB-97 | BR00-050 [symbol] | 9830-0200 | BR95-003 [symbol] |
| | Class | | | | | | | | | |
| Symbol | LUMINAL | LUMINAL | LUMINAL | LUMINAL | LUMINAL | LUMINAL | LUMINAL | LUMINAL | LUMlNAL | LUMINAL |
| | 1.4335 | 0.3065 | 1.3685 | 0.8185 | 0.0145 | 0.6765 | 0.3445 | 1.8895 | −0.1345 | 1.6815 |
| | 0.356 | 0.014 | −0.439 | −0.07 | 0.034 | 0.114 | 0.643 | 0.073 | 0.476 | 0.506 |
| ABAT | −0.586 | 1.213 | 1.232 | 2.379 | 2.715 | 1.713 | 0.006 | 0.483 | 2.035 | −1.507 |
| ABC1 | 0.8675 | 0.3105 | −0.1075 | 0.5455 | 2.3455 | 3.1885 | 0.2585 | −0.5615 | 1.3465 | −0.2155 |
| ABCC11 | 1.0505 | 0.4745 | −0.1095 | 0.2055 | 0.5735 | 0.5625 | 0.8875 | −0.5525 | −0.3265 | −0.5065 |
| ABCC3 | −0.155 | 0.316 | 0.526 | 0.109 | −0.086 | −0.495 | −0.348 | 1.422 | −1.127 | 0.276 |
| ACAS2L | −0.0485 | −0.4465 | −0.3785 | 0.1805 | 0.1625 | −0.5315 | −0.5305 | −0.1675 | 0.1975 | −0.0245 |
| ACAT1 | −0.655 | 1.48 | 0.379 | 0.854 | 1.311 | −0.35 | 0.367 | 3.48 | 2.313 | 1.559 |
| ACAT2 | 0.0055 | −0.4515 | 0.4225 | 0.1545 | 0.2115 | 0.6785 | 0.7105 | 1.2195 | −0.0195 | −0.1905 |
| ACOX2 | −0.1395 | 0.0805 | 0.9935 | 0.9925 | 0.2165 | 0.9825 | −0.0095 | 0.0375 | 0.7395 | −0.0695 |
| ACDX3 | 3.4175 | −0.4895 | −0.2375 | −0.0165 | −1.3605 | −0.6535 | −0.4455 | −0.9055 | −0.5525 | −0.5715 |
| ACSL3 | −0.339 | 0.048 | −0.534 | −1.008 | −0.038 | 0.682 | −0.038 | −0.284 | −1.284 | −1.083 |
| ACTA1 | 0.0575 | −0.2495 | −0.0535 | 0.0705 | −0.2585 | −0.4155 | −0.1176 | 0.3055 | −0.4145 | 0.0175 |
| ACTB | −0.1726 | −0.9295 | −1.0025 | −0.7215 | −0.2415 | −0.3345 | −0.9886 | −0.8395 | −0.3885 | −1.2545 |
| ADAM33 | −0.846 | −0.834 | −0.169 | −0.171 | −0.139 | −0.586 | −1.261 | 0.891 | −1.39 | 0.336 |
| ADAMDE [symbol] | 0.2535 | 0.0055 | 0.7405 | −0.2835 | 0.4095 | 0.6585 | 0.4695 | 0.8385 | 1.7925 | 0.1285 |
| ADPN | −0.8755 | −0.0625 | −0.1255 | −0.0765 | 0.2065 | 2.5335 | −0.1415 | −0.4475 | −1.5795 | −0.8275 |
| ADPRHL1 | −0.7375 | 0.0185 | −1.6165 | 0.0925 | 1.1685 | −0.3195 | 0.1175 | 0.3245 | 0.0905 | −1.2055 |
| ADRA2C | 0.2185 | 0.2505 | −0.3125 | −0.1325 | −0.4825 | −0.2915 | −0.0415 | 0.2115 | 1.1545 | 1.8565 |
| AGR2 | 0.7235 | 3.5985 | −0.0365 | 0.1485 | 0.3005 | −0.1105 | 0.8855 | 0.4445 | 0.1905 | 2.8595 |
| AGT | 0.145 | 0.237 | 0.299 | −0.094 | 0.053 | 0.191 | 0.454 | 0.581 | 0.574 | 0.332 |
| AGTR1 | 0.477 | 0.208 | −0.152 | 0.663 | 0.507 | 0.535 | −0.06 | 0.931 | −0.732 | −0.656 |
| AHCYL1 | 0.577 | −0.348 | 0.32 | −0.264 | −0.121 | 0.606 | −0.002 | −0.932 | −0.537 | −0.728 |
| AHNAK | −0.858 | 0.763 | −0.899 | 0.908 | 0.607 | 1.172 | −0.543 | −0.163 | −0.355 | −1.508 |
| AHR | −0.2195 | −0.0905 | 0.3125 | −0.3095 | −0.1565 | −0.3385 | 0.1205 | 0.0025 | 0.1195 | 0.1065 |
| AIM1 | 0.1915 | 0.7395 | −0.0005 | −0.0445 | −0.1115 | −0.5045 | 0.0285 | 0.4785 | −0.0345 | 0.1035 |
| AK2 | 2.7645 | 2.7445 | −0.1025 | 2.9445 | 1.7345 | 2.1585 | 1.4745 | 4.0095 | 3.2975 | 3.0605 |
| AK3L1 | 0.33 | −0.489 | −0.441 | −0.028 | −0.096 | 0.114 | 0.565 | −0.544 | 0.449 | 0.046 |
| AK5 | 0.048 | −0.277 | 0.306 | 0.124 | 0.194 | −0.468 | −0.139 | −0.242 | 0.445 | −0.026 |
| AKAP1 | 0.4425 | 0.6505 | 0.3015 | 0.2625 | 0.7735 | −0.5655 | 0.1565 | 1.5815 | 1.0425 | −0.5255 |
| AKR1A1 | 0.475 | 0.084 | 0.449 | 0.032 | −0.07 | 0.042 | −0.079 | −0.049 | 0.393 | −0.463 |
| AKR7A3 | 0.3875 | −0.2515 | −0.1595 | −0.1015 | 0.0585 | −0.4825 | −0.4245 | −0.4765 | −0.0475 | −0.0865 |
| ALG5 | 1.3115 | 0.3265 | 0.8605 | −0.3395 | 0.8515 | −1.7475 | −0.7655 | 1.5145 | −0.5545 | −0.9515 |
| ALG8 | 0.0845 | 0.4375 | 0.1885 | −0.2535 | −0.0845 | 0.2965 | 0.1445 | 0.3035 | −0.0875 | −0.1575 |
| AMFR | 0.348 | −0.223 | −0.534 | −0.579 | 0.245 | −0.555 | 0.489 | 0.325 | 0.729 | 0.334 |
| AMPD2 | −0.469 | −0.657 | 0.094 | −0.535 | 0.974 | −0.296 | −1.087 | 0.052 | −1.815 | −0.536 |
| ANAPC11 | −0.36 | −0.085 | −0.126 | 0.009 | −0.25 | 0.188 | −0.114 | −0.313 | −1.205 | −0.926 |
| ANKRD22 | −1.504 | −0.583 | −1.081 | −1.729 | −0.852 | −1.189 | 0.053 | −0.784 | −0.478 | −0.404 |
| ANKRD27 | −0.179 | 0.063 | −0.185 | 0.295 | −0.028 | −0.249 | 0.217 | 0.008 | 0.001 | −0.219 |
| ANLN | 0.019 | −0.292 | −1.265 | 0.944 | 0.91 | 0.305 | −0.419 | −1.268 | −2.743 | −3.115 |
| ANP32A | −0.329 | 0.261 | −0.146 | −0.089 | 0.494 | 1.153 | 0.938 | 0.632 | 0.845 | 0.099 |
| ANXA3 | −1.1575 | −1.1095 | −1.3825 | −1.1725 | −1.8285 | −0.8305 | 0.4805 | −0.2325 | −0.7815 | −0.2085 |
| APBB2 | −0.4985 | −0.1495 | −0.6575 | 0.8955 | 0.6925 | 0.5885 | 1.4545 | −0.5345 | 4.4635 | −1.0505 |
| APOBEC3 | −0.298 | 1.097 | 0.827 | 0.399 | 0.852 | −0.153 | 0.851 | 0.152 | 0.418 | −0.251 |
| AQP5 | 0.244 | 0.668 | 2.341 | 2.653 | 3.631 | 1.965 | 0.616 | −0.231 | −0.435 | 1.798 |
| AR | −0.0165 | −0.7295 | −0.3385 | −1.0555 | −0.5925 | −0.2845 | −0.1685 | −0.3965 | 0.2335 | 0.0375 |
| AREG | 1.093 | 0.175 | 0.964 | 0.302 | −0.55 | −0.011 | 0.521 | 0.446 | −0.836 | −0.508 |
| ARF1 | −0.0895 | −0.1245 | −0.2085 | −0.3895 | −0.4495 | −0.4105 | 0.3405 | 0.2195 | −0.0285 | 0.3225 |
| ARHGDI [symbol] | −0.303 | 0.009 | −0.095 | 0.137 | 0.377 | −0.149 | −0.122 | −0.853 | −0.704 | −0.673 |
| ARL6IP | 0.3325 | 0.1885 | 0.2535 | 0.5335 | −0.0385 | 0.0885 | 0.0035 | −0.2315 | 0.0005 | 0.1595 |
| ARL6IP2 | 0.57 | 1.652 | 1.189 | −0.861 | −0.325 | 0 | 0.482 | 0.007 | 1.023 | 1.488 |
| ARMCX2 | 0.593 | 1.469 | 1.079 | −0.633 | −0.18 | 0.091 | 0.513 | 0.474 | 0.989 | 1.396 |
| ARNT2 | −0.26 | −0.448 | 0.365 | 0.014 | 0.253 | −0.417 | −0.314 | −0.445 | −0.808 | −0.022 |
| ARNT2 | −0.1125 | 0.0245 | −0.0575 | −0.8855 | −0.5095 | 0.1125 | −0.1826 | 0.2305 | −0.2435 | −0.7465 |
| ARP3BET [symbol] | 0.3445 | −0.1415 | 0.0775 | −0.3785 | −0.3075 | −0.2365 | −0.1875 | −0.1955 | 0.0905 | −0.3515 |
| ARPC1B | −0.556 | 0.018 | 0.06 | 0.159 | −0.108 | 1.934 | −0.217 | −0.52 | −1.182 | −0.15 |
| ARPC5L | −0.3635 | −0.0555 | 0.3025 | −0.1535 | −0.5115 | −0.6725 | −0.5445 | −0.2275 | 0.8476 | −0.4015 |
| AS89 | −0.336 | 0.12 | −0.185 | −0.325 | −0.005 | −0.34 | 0.434 | 0.145 | −0.155 | 0.017 |
| ASF1A | −0.8875 | −0.7505 | −0.2515 | −0.5375 | −1.0075 | −1.1935 | −0.7125 | −0.4275 | −0.2125 | −0.9075 |
| ASH2L | −1.063 | −1.177 | −0.066 | −1.546 | −1.268 | −1.241 | 0.133 | −1.499 | 0.607 | 0.014 |
| ASK | −0.34 | −0.339 | 0.003 | 0.165 | −0.066 | 0.16 | −0.204 | −0.921 | 0.085 | −0.227 |
| ASPM | −0.8955 | −0.5225 | −1.1435 | −0.8355 | −0.3965 | −0.7995 | −0.2445 | −0.0155 | −0.4365 | −0.3155 |
| ASS | −0.2505 | 0.0495 | −0.2035 | −0.1485 | −0.1815 | 0.3855 | 0.5245 | −0.0295 | −0.0845 | −0.4775 |
| ATAD2 | 0.2765 | −0.5765 | 0.5875 | −0.6945 | −0.1475 | 0.3725 | 0.9955 | −1.1355 | 0.3515 | −1.3955 |
| ATF5 | −0.867 | −0.09 | −0.119 | 0.009 | 1 | 0.619 | 0.518 | −0.815 | −0.584 | −0.792 |
| ATF7IP2 | 0.019 | −0.243 | 0.028 | −0.412 | 0.942 | −0.045 | 0.353 | −0.131 | 0.581 | 0.118 |
| ATP2B1 | −0.269 | 0.012 | 0.058 | −0.228 | 0.209 | −0.253 | 0.022 | −0.258 | 0.261 | −0.241 |
| ATP5G1 | 0.2395 | 0.2045 | −0.0195 | −0.5545 | 0.2285 | 0.2425 | 0.1355 | 0.1405 | 0.1325 | 0.4015 |
| ATP5O | 2.126 | −0.668 | 0.15 | 0.199 | 0.136 | 1.684 | −0.646 | −0.963 | 0.656 | −0.706 |
| ATP6AP1 | −0.072 | 0.387 | 0.126 | 1.151 | 0.152 | −0.485 | −0.446 | −0.511 | −0.13 | 0.473 |
| ATP6V0A4 | −0.701 | 0.488 | 0.47 | 0.024 | 0.29 | 0.003 | 0.328 | 0.058 | 0.233 | −0.1 |
| ATP6V1B [symbol] | −0.1135 | −0.0525 | 0.4095 | −0.0855 | −0.2665 | −0.4395 | 0.5235 | 0.4585 | 0.2305 | 0.2125 |
| ATP6V1G [symbol] | 0.249 | 0.117 | 0.286 | −0.224 | −0.294 | −0.206 | −0.264 | 0.233 | 0.149 | 0.211 |
| ATPIF1 | 0.2885 | 0.1685 | −0.5595 | 0.1325 | −0.6685 | −0.7865 | −0.5785 | −1.4355 | −1.0275 | −1.5195 |
| ATXN10 | −0.416 | 0.038 | −0.103 | −0.32 | 0.455 | −0.144 | 0.444 | −0.38 | −1.774 | −0.766 |
| B3GNT5 | 0.8655 | 0.0005 | 0.4945 | −0.1745 | 0.0575 | 0.7305 | 0.3065 | 0.5685 | 0.2625 | 1.1195 |
| BACE2 | −0.0235 | −0.0345 | 0.1945 | 0.0585 | −0.1466 | −0.1625 | −0.3825 | 0.6825 | −0.0805 | −0.1405 |
| BAG1 | 0.5035 | 0.7255 | 0.8055 | −0.4725 | −0.4225 | 1.1315 | 1.5075 | 0.5975 | 1.5795 | 1.4295 |
| BAG4 | −2.5625 | −2.8175 | 0.1155 | 0.0665 | −0.5235 | 1.4315 | 0.2075 | −3.0305 | −0.4695 | −3.0655 |
| BAI2 | −0.388 | 1.237 | −0.05 | −0.545 | −0.408 | 0.481 | 1.249 | 1.151 | −1.24 | 0.08 |
| BAMBI | −0.362 | −0.38 | −0.104 | −0.084 | 0.07 | −0.33 | −0.31 | −0.065 | −0.382 | −0.252 |
| BCAS1 | 2.605 | 0.953 | 0.738 | −0.013 | −0.966 | 0.418 | 0.811 | 0.461 | 1.336 | 0.636 |
| BCL2L14 | 3.0515 | 3.6825 | 3.2015 | 0.9325 | 0.8355 | 0.0705 | 4.2365 | 3.4035 | −2.1795 | 2.1145 |
| BCMP11 | 0.821 | 1.787 | 1.315 | 0.965 | 0.193 | −0.206 | 2.192 | 2.26 | −2.74 | 1.266 |
| BEX1 | 0.7545 | 1.1895 | 1.0035 | −0.6705 | −1.1625 | −1.2345 | 0.7895 | 1.7355 | −0.9675 | 1.3465 |
| BEX2 | 0.9515 | −1.0515 | −0.1275 | −0.2895 | 0.3155 | −0.7225 | −0.4835 | −0.7765 | −0.0475 | 0.2195 |
| BF | −0.356 | 0.748 | −0.141 | 0.026 | 0.826 | −0.095 | 1.136 | 1.11 | 1.036 | −0.277 |
| BFSP1 | 0.302 | 0.056 | 0.507 | −0.137 | −0.122 | −0.241 | −0.413 | −0.219 | −1.121 | −0.202 |
| BIK | −1.715 | −1.02 | −1.319 | −1.681 | −0.406 | −0.982 | 0.708 | −0.415 | −0.242 | −0.857 |
| BIRC2 | 0.0355 | −0.2215 | −0.1735 | −0.4865 | −0.4915 | −0.2445 | 0.0535 | −0.1385 | −0.0315 | 0.0645 |
| BIRC5 | −0.2415 | −0.0905 | −0.7865 | 0.0305 | −0.0515 | −0.2275 | −1.0085 | 0.3116 | −0.4615 | −0.2615 |
| Bit1 | −0.1885 | 0.1575 | −0.0385 | −0.3195 | 0.0255 | 0.1655 | 0.3425 | 0.8305 | −0.1805 | −0.6035 |
| BIVM | −1.077 | −0.63 | −0.608 | −0.671 | −0.692 | −0.706 | −0.559 | 0.694 | 0.288 | −0.822 |
| BLVRA | 0.13 | −1.022 | 0.415 | 0.079 | 0.713 | 0.53 | 0.153 | 0.33 | −0.977 | −0.706 |
| BM039 | −0.048 | 0.66 | 0.135 | 0.32 | 1.797 | 1.139 | 1.1 | −0.485 | −0.014 | −0.115 |
| BMP7 | 0.118 | −0.217 | −0.44 | −0.534 | −0.715 | 0.498 | 0.296 | 0.036 | 0.641 | 1.056 |
| BOC | −0.5165 | −0.1975 | 0.1305 | −0.3575 | −0.1925 | −0.0635 | −0.0475 | −0.0095 | 0.1115 | −0.2895 |
| BRCA1 | 0.1715 | −0.3445 | −0.4985 | −0.0375 | −0.6165 | 0.1025 | −0.1675 | −0.7065 | 0.0605 | 0.1525 |
| BRF2 | −1.207 | −0.852 | −0.355 | −1.085 | 0.016 | −0.185 | 0.319 | 1.322 | 0.88 | 0.01 |
| BRI3 | −0.97 | −0.233 | 0.811 | 0.227 | −0.274 | −0.486 | −0.051 | 0.452 | 0.880 | 0.029 |
| BRI3BP | −0.36 | 0.716 | 0.156 | 0.197 | 0.207 | −0.047 | 0.255 | 0.171 | 0.026 | 0.012 |
| BSPRY | 0.2855 | −0.3705 | −0.5315 | −0.5275 | −0.4205 | −0.8025 | −0.6955 | −0.0045 | −0.4235 | 0.1495 |
| BTF3 | 0.4265 | −0.4735 | −0.0225 | −0.3495 | −0.4145 | −0.3965 | −0.3955 | −0.0485 | −0.4375 | 0.0425 |
| BTG3 | −0.76 | −0.903 | −0.596 | −0.724 | −1.149 | −1.443 | −0.198 | −1.119 | −0.274 | −0.336 |
| BγN2A2 | −0.045 | −0.143 | −0.082 | −0.626 | −0.031 | −0.672 | −0.502 | −0.892 | 0.536 | −0.158 |
| BUB1 | −0.3775 | 0.0015 | −0.3275 | −0.1925 | −0.1335 | −0.1775 | −0.1105 | 0.1015 | 0.3325 | −0.7445 |
| BUB3 | −0.24 | −0.175 | −0.233 | −0.039 | 0.164 | −0.138 | −0.273 | −0.252 | −0.112 | −0.645 |
| BXDC1 | 0.242 | −0.052 | 0.592 | 0.1 | −0.154 | 0.404 | −0.135 | 0.121 | −0.043 | −0.035 |
| BZRP | −0.005 | 0.207 | −0.684 | 0.646 | 0.685 | 1.152 | 0.502 | 0.735 | 1.195 | 0.204 |
| BZW1 | −0.1155 | 0.2735 | −0.0185 | 0.3135 | 0.2915 | 0.3965 | 0.3515 | −1.2565 | −0.5795 | 0.0505 |
| C10orf116 | −0.5415 | 0.4645 | 0.1225 | 0.0555 | 0.0865 | 0.1635 | 0.5885 | −0.0765 | 0.0105 | −0.2255 |
| C10orf38 | 0.013 | 0.701 | 0.229 | −1.197 | −0.19 | 1.063 | −0.377 | 1.569 | −2.309 | −1.138 |
| C10orf4 | −0.2135 | 0.0175 | −0.0735 | −0.3105 | −0.0255 | −0.2085 | −0.1455 | −0.2745 | −0.1485 | −0.2685 |
| C10orf45 | −0.4105 | −0.3525 | 0.4155 | −0.2735 | 0.4876 | 1.7675 | −1.3425 | 1.0675 | −0.3985 | −0.7005 |
| C10orf7 | 0.1925 | 0.4755 | −0.2075 | −0.4445 | −0.2195 | 0.1665 | −0.3225 | −0.2475 | 0.8305 | −0.3245 |
| C10orf82 | −0.685 | −0.843 | −0.3 | −0.216 | 0.678 | −0.241 | 0.432 | −0.853 | −0.454 | −0.912 |
| C11orf24 | 0.2625 | 1.2455 | 0.3945 | 0.4235 | 0.7455 | 0.1705 | 0.3975 | 1.5405 | 0.7045 | 0.3185 |
| C12orf14 | −0.107 | −0.658 | −0.197 | −0.4 | −0.323 | −0.689 | −0.455 | −0.129 | −0.242 | −0.787 |
| C13orf21 | 0.062 | −0.108 | −0.515 | −0.019 | 0.389 | 0.084 | 0.281 | 0.28 | 0.019 | 0.057 |
| C13orf23 | −0.784 | −0.586 | −0.305 | −0.282 | −0.545 | 0.044 | −0.742 | −0.776 | −0.112 | −0.55 |
| C14orf94 | 0.027 | 1.01 | 0.88 | 0.564 | 0.26 | 2.39 | 1.27 | 2.271 | 1.98 | 1.237 |
| C15orf23 | −0.0715 | −0.0485 | −0.1285 | −0.3705 | 1.3465 | 0.2025 | −0.1615 | −0.3375 | 0.4545 | 0.5665 |
| C16orf45 | −0.548 | 0.216 | 1.195 | 0.018 | −0.551 | 0.173 | 0.168 | 0.753 | −0.371 | −0.425 |
| C17orf37 | −0.297 | 0.049 | −0.041 | −0.513 | −0.285 | −0.382 | −0.251 | −0.316 | 0.303 | −0.311 |
| C18orf1 | 0.256 | 0.21 | 0.388 | 0.458 | 0.341 | −0.495 | 0.2 | 1.199 | 0.939 | 0.557 |
| C19orf10 | 0.4135 | 0.6275 | 0.3455 | 0.0065 | 0.1805 | 1.4595 | −0.1925 | 1.0825 | 0.6185 | 1.1005 |
| C19orf32 | 0.7875 | 0.8245 | −0.5955 | 0.8095 | 0.8016 | 0.5055 | 1.2665 | −0.6005 | −0.2485 | −0.7525 |
| C19orf33 | 0.751 | −0.155 | 0.704 | 0.212 | 0.085 | −0.234 | 0.179 | 0.653 | 1.68 | −0.477 |
| C1orf24 | 1.182 | 0.077 | 1.652 | −1.011 | 0.107 | 1.508 | 1.208 | −0.911 | −0.069 | 0.576 |
| C1orf34 | −0.1895 | −0.0485 | −0.2475 | −0.2725 | −0.0195 | 0.8215 | 0.4235 | 0.0455 | 0.1775 | 0.0195 |
| C20orf103 | 2.5845 | 0.2805 | 0.6955 | 1.1095 | 0.1705 | 0.0845 | −0.3605 | 1.1585 | 2.2605 | 1.2005 |
| C20orf149 | 1.298 | −0.12 | 0.657 | −0.043 | −0.053 | −0.088 | 0.191 | 0.088 | 0.483 | 0.028 |
| C20orf23 | −0.275 | 0.572 | −0.172 | −0.454 | −0.549 | 0.621 | 0.174 | 0.378 | 0.042 | −0.089 |
| C20orf3 | −0.164 | −0.415 | −0.004 | −0.378 | −0.257 | 0.273 | −0.073 | −0.241 | −0.392 | −0.152 |
| C20orf35 | −0.187 | −0.512 | −0.044 | −0.107 | −0.521 | 0.013 | −0.76 | 0.036 | 0.122 | −0.354 |
| C20orf43 | −0.4765 | −0.6595 | −0.2425 | −0.4695 | −0.4305 | 0.2305 | 0.0985 | 0.1855 | −0.0655 | −0.4345 |
| C20orf8 | −0.603 | −0.604 | −0.925 | −0.879 | −0.331 | −0.262 | 0.786 | −0.071 | 0.34 | 0.658 |
| C21orf45 | −0.252 | −0.246 | 0.042 | −0.383 | −0.148 | −0.291 | 0.136 | 0.264 | −0.646 | −0.252 |
| C22orf18 | 0.072 | −0.037 | 0.007 | 0.013 | −0.265 | −0.106 | 0.123 | 0.216 | −0.096 | 0.044 |
| C2F | 1.104 | 1.184 | 1.043 | 0.298 | 1.175 | 0.521 | 0.555 | 1.962 | 0.946 | 0.825 |
| C2orf17 | 0.512 | 0.656 | 1.275 | −1.26 | −1.087 | −0.372 | 0.174 | −0.522 | −0.976 | −0.368 |
| C4A | −0.078 | −0.517 | −0.22 | −0.348 | −0.174 | −0.143 | −0.304 | −0.314 | 0.378 | −0.354 |
| C5orf13 | −0.235 | −0.086 | −0.464 | −0.208 | −0.024 | 0.661 | −0.034 | 0.028 | 0.221 | −0.492 |
| C6orf108 | 0.152 | 0.522 | 0.218 | 0.083 | −0.872 | −0.441 | −0.326 | −0.358 | −0.303 | 1.115 |
| C6orf166 | 0.325 | 2.446 | 0.489 | 0.93 | 0.169 | −1.19 | 1.005 | 0.852 | 1.077 | 2.521 |
| C6orf192 | −0.1015 | −0.2835 | 0.0685 | −0.1576 | 0.0695 | 0.2385 | 0.1485 | −0.2375 | 0.1675 | 0.0095 |
| C6orf211 | 0.057 | −0.147 | −0.107 | 0.228 | 0.853 | −0.284 | −0.218 | −0.391 | −2.245 | −1.782 |
| C6orf49 | −0.4415 | −0.3835 | 0.8045 | −0.0895 | 0.2205 | −0.2645 | −0.8175 | −0.9205 | 1.0535 | −0.2735 |
| C6orf85 | −0.5345 | 0.6465 | −0.3435 | −0.0365 | 0.5935 | 0.9745 | 0.7205 | 0.5225 | 0.2135 | 0.5515 |
| C7orf24 | −0.148 | 0.78 | 0.592 | 1.155 | 0.27 | 1.213 | 1.313 | 0.989 | 0.407 | −0.433 |
| C9orf10 | −2.106 | −1.002 | −0.174 | 0.616 | 0.544 | 0.992 | 0.256 | −0.412 | −2.674 | −1.746 |
| C9orf121 | −0.9 | −0.013 | −0.03 | −0.345 | 0.41 | 0.105 | 0.69 | −0.084 | 0.276 | −0.058 |
| C9orf58 | 0.0845 | −0.1695 | 0.7425 | 0.1595 | 0.3655 | 0.1995 | 0.1065 | 0.8135 | 2.1065 | −0.1135 |
| C9orf88 | 0.475 | 0.202 | −0.606 | −0.419 | 0.346 | 1.881 | 1.078 | 1.963 | 0.866 | 1.002 |
| C9orf9 | 0.03 | −0.263 | −0.12 | 0.444 | 0.617 | −0.067 | −0.636 | −0.046 | 0.477 | −0.423 |
| CA12 | 0.082 | −0.408 | −0.034 | 0.57 | 0.499 | −0.337 | −1.124 | −0.093 | 0.205 | −0.574 |
| CA8 | 0.1 | 0.241 | −0.322 | 0.531 | 1.091 | 0.416 | 0.378 | 0.003 | 0.86 | 0.11 |
| CA8 | −0.0725 | −0.1215 | −0.0975 | −0.0775 | −0.1615 | 0.0195 | −0.1115 | −0.0045 | 3.3285 | 0.0915 |
| CACNA1G | −0.211 | −0.038 | −0.245 | 0.07 | −0.232 | −0.171 | −0.145 | −0.332 | −1.072 | −0.21 |
| CALCA | −0.528 | −1.021 | −0.298 | 0.138 | 1.202 | −0.65 | −0.397 | 0.223 | −0.767 | −0.588 |
| CALML4 | −0.8725 | −0.5735 | 0.2995 | 0.7115 | 0.5185 | 0.4035 | −0.5715 | −0.3385 | −0.4495 | −0.0745 |
| CALML5 | −0.0065 | −0.1915 | −0.2405 | 0.4145 | 1.2485 | 0.7785 | 2.3235 | 0.3195 | −0.3535 | 1.0625 |
| CaMKIINa [symbol] | 0.016 | −0.023 | −0.206 | −0.346 | −0.22 | −0.489 | 0.471 | −0.499 | 0.946 | 0.191 |
| CAP2 | −0.815 | 0.826 | 0.735 | −0.315 | 0.263 | −0.239 | 0.194 | 1.554 | 1.771 | 0.518 |
| CAP350 | 0.2865 | 1.0065 | 0.8235 | 1.2355 | 2.7415 | 0.3055 | 0.9785 | −0.4035 | −1.2085 | −0.0705 |
| CAPN13 | 0.356 | 0.33 | −0.007 | 0.297 | 0.251 | 0.387 | 2.014 | 0.629 | 0.929 | −0.275 |
| CAPN9 | 0.0505 | 4.4155 | 2.9835 | −0.0305 | 0.0785 | 1.8535 | 0.3735 | 1.1475 | 6.8185 | 4.7175 |
| CARD14 | 0.2085 | −0.3245 | 0.3195 | −0.0505 | 0.4945 | −0.3925 | 0.0115 | −1.7215 | −1.3695 | −1.1845 |
| CART | −0.4135 | −0.0205 | −0.5535 | −0.4785 | −0.2325 | 0.1745 | −0.0445 | −0.4425 | 0.3085 | −0.6755 |
| CASP1 | 1.44 | 0.002 | −0.066 | −0.718 | −0.584 | −0.517 | −0.148 | 0.168 | −0.974 | −0.853 |
| CASP3 | 0.045 | 0.205 | 0.841 | 0.626 | −0.029 | 0.285 | 0 | 0.102 | −0.059 | 0.865 |
| CASP7 | −0.4925 | −0.4155 | −0.4555 | 0.1585 | −0.1355 | −0.4155 | −0.2955 | −1.0725 | −2.1855 | −1.9245 |
| C9LN1 | −1.109 | −0.411 | 0.324 | −0.024 | 0.264 | −0.224 | −0.402 | −1.42 | 2.156 | 0.878 |
| CBR1 | −0.2085 | 0.4895 | −0.4875 | −0.1575 | 0.5285 | −0.3165 | −0.5045 | 0.5275 | −0.0485 | −0.1166 |
| CBR3 | −0.061 | 0.216 | 0.067 | −0.037 | 0.19 | 0.173 | 0.412 | 0.018 | 0.12 | −0.033 |
| CBR4 | −1.0115 | −0.4445 | 0.8695 | 2.3025 | 1.8425 | 1.1315 | 1.1835 | 0.1055 | 0.1425 | −0.0025 |
| CCL24 | −0.911 | −1.086 | −0.671 | −1.027 | −0.642 | −0.427 | −0.39 | −0.574 | −0.522 | −0.502 |
| CCL29 | 0.196 | 0.337 | −0.307 | −0.629 | −0.22 | −0.84 | −0.523 | 1.091 | 3.047 | 0.543 |
| CCNB2 | −0.1795 | −0.5265 | 0.0225 | 0.0005 | −0.5635 | −0.2055 | −0.2405 | 0.0665 | −0.3525 | −0.3345 |
| CCND1 | 1.508 | 1.026 | 0.182 | 0.883 | 0.607 | 0.683 | 0.099 | 0.279 | −0.802 | −0.07 |
| CCNE1 | −0.1075 | 0.3045 | 0.1855 | 0.0765 | −0.1925 | −0.3285 | −0.3335 | 0.5865 | 0.1835 | 0.3065 |
| CCNG2 | −0.344 | −0.595 | 0.061 | −0.017 | −0.658 | −0.88 | −0.488 | −0.357 | −0.68 | −0.927 |
| CCNH | −0.1785 | −0.4325 | 0.7245 | −0.1155 | −0.2155 | −1.0385 | −0.2535 | −0.1585 | −1.3655 | −0.8825 |
| CD38 | −0.1345 | −0.1855 | 0.0145 | 0.0255 | −0.0475 | −0.0575 | −0.0285 | −0.1285 | 0.0085 | −0.2785 |
| C030 | −0.0135 | −0.4425 | 0.2575 | −0.1285 | −0.7125 | −0.7385 | −0.7075 | −0.3515 | 0.2045 | 0.1345 |
| CD4 | 1.4005 | 0.2605 | −0.3915 | 0.7035 | 0.5875 | 0.9155 | −0.1825 | 0.2425 | −0.0305 | −0.1315 |
| CD58 | −1.506 | −0.832 | −0.453 | −2.105 | −1.329 | −0.544 | −0.473 | −0.75 | 0.195 | −0.278 |
| CD50 | −1.3445 | −1.2025 | −0.4545 | −1.2315 | −1.0435 | −0.8535 | −0.0035 | 0.0035 | 0.5975 | −0.8085 |
| CDC2 | −1.4155 | −1.4345 | −0.8775 | −1.9315 | −1.5545 | −1.4105 | −0.0085 | −1.2515 | 1.1495 | 2.8185 |
| CDC6 | −1.5825 | −1.4685 | −1.3485 | −1.9085 | −0.9715 | −1.5005 | −0.4245 | −0.5095 | −1.4965 | −0.8885 |
| CDCA1 | −0.883 | −0.591 | −0.384 | −0.02 | 0.096 | −0.452 | −0.628 | −0.562 | −1.426 | −0.183 |
| CDCA5 | −0.57 | −0.031 | 0.461 | 0.053 | 0.802 | 1.241 | −1.608 | 0.748 | 0.515 | −0.437 |
| CDCA7 | 0.7625 | −0.2725 | −0.4875 | 0.0775 | 1.8685 | 1.3185 | 0.7665 | 0.1905 | −1.1625 | −1.2955 |
| CDH1 | −1.099 | −0.233 | −1.072 | −0.509 | −0.282 | 0.035 | 0.638 | 0.464 | −0.573 | −0.874 |
| CDH3 | 0.2805 | −0.6465 | 0.3775 | −0.2775 | 0.0075 | −0.0655 | 0.1705 | −0.0265 | −0.1815 | 0.1995 |
| CDK2 | −0.3685 | −0.3835 | −0.3325 | −0.6485 | −1.4835 | −1.0955 | −0.0635 | −0.3605 | 0.2715 | 0.1675 |
| CDKN2A | −1.3355 | −0.4235 | −0.7605 | −1.5755 | −0.7545 | −0.8655 | −0.3575 | −0.3595 | 0.0625 | −0.4265 |
| CDKN2D | −1.46 | −0.677 | −0.839 | −1.08 | −0.093 | −1.135 | 0.028 | 0.548 | 0.358 | −0.882 |
| CDKN3 | −0.439 | −0.416 | −0.057 | 0.221 | −0.224 | −0.32 | −0.385 | −0.552 | −0.771 | −0.13 |
| CDT1 | 1.2495 | 1.5605 | 0.5805 | −0.0545 | 0.0135 | 0.1245 | 1.5465 | 1.5665 | 2.0975 | 1.4485 |
| CE9PG | −1.175 | −1.135 | −0.989 | −0.969 | −1.061 | −0.976 | −0.731 | −0.488 | −0.045 | −0.81 |
| CELSR1 | −0.296 | −0.498 | −0.273 | −0.418 | −0.755 | −0.585 | 0.189 | −0.626 | −0.163 | 0.024 |
| CENPA | −1.03 | −1.742 | −1.003 | −2.232 | −1.312 | −1.254 | 0.232 | −1.392 | 0.068 | −0.272 |
| CENPE | −0.292 | 0.009 | −0.167 | −0.287 | 0.351 | 0.378 | 0.432 | 0.114 | −0.062 | −0.273 |
| CENPF | 0.2375 | 0.3165 | −0.0015 | 0.5555 | 0.8855 | 0.9355 | 0.2785 | 1.9035 | 0.5645 | −0.2305 |
| CENTG3 | −0.5345 | 0.1815 | −0.0995 | 0.1715 | −0.3276 | −0.5865 | −0.5325 | 0.1205 | 0.3905 | 0.1265 |
| CES2 | 0.497 | 0.078 | −0.529 | 0.535 | −0.485 | 1.802 | −0.383 | −0.63 | −0.626 | 0.316 |
| CETN3 | 0.4605 | −0.8495 | −0.0715 | −0.7805 | −0.7515 | −0.3415 | −0.8765 | 0.1865 | −0.5425 | −0.6185 |
| CGA | 0.4845 | 0.1435 | −0.2515 | −0.5565 | 1.6605 | −0.3115 | 0.6795 | −0.2235 | 0.3775 | 0.3755 |
| CGI-12 | 0.7505 | −0.1695 | −0.2535 | −0.4665 | −0.8715 | 0.8335 | 0.2705 | 0.7125 | −0.3795 | 0.7995 |
| CGI-49 | 0.047 | 0.159 | −0.314 | −0.134 | −0.553 | 0.703 | 1.941 | 0.327 | −0.011 | −0.211 |
| CGI-4 [symbol] | −0.08 | 1.151 | 0.081 | 1.584 | 1.441 | 0.293 | 0.662 | 1.761 | 0.158 | −0.19 |
| CGN | −0.307 | −1.348 | −0.307 | −0.387 | −0.952 | −1.16 | 0.167 | −0.337 | −0.241 | −0.127 |
| CGNL1 | −2.0765 | −0.2695 | 0.2685 | −0.9765 | 0.0005 | −0.1225 | 0.4025 | −1.5945 | −0.5025 | −1.7395 |
| CHAF1B | −0.602 | −0.125 | −0.003 | −0.689 | −0.074 | 0.237 | 0.129 | −0.028 | −0.121 | −0.532 |
| CHC1 | 0.161 | −0.391 | 0.166 | 0.041 | 0.332 | 0.537 | 0.348 | 0.79 | 0.689 | −0.237 |
| CHCHD2 | 1.741 | 0.113 | −0.699 | 2.852 | 1.341 | 1.203 | −0.001 | −1.696 | 2.385 | −1.013 |
| CHCH05 | 1.391 | −0.885 | 1.264 | 0.92 | 0.386 | −0.365 | −0.518 | −1.496 | 0.4 | 1.318 |
| CHI3L1 | −0.44 | 0.071 | 0.055 | −0.183 | 0.402 | 0.102 | 0.013 | 0.136 | 0.908 | −0.314 |
| CHI3L2 | 0.014 | 0.018 | −0.091 | −0.008 | −0.022 | 0.062 | −0.055 | −0.003 | −0.218 | 0.061 |
| CHKA | 0.312 | 0.314 | 0.159 | −0.057 | 0.024 | 0.322 | −0.062 | 0.141 | 1.009 | 0.299 |
| CHCDL | 0.2145 | 0.0315 | −0.0165 | 0.1016 | −0.2095 | 0.2115 | 0.0305 | 0.0275 | −0.0045 | 0.0665 |
| CHRDL2 | −0.018 | 0.145 | −0.199 | 0.122 | 0.03 | 0.002 | −0.018 | −0.111 | 0.893 | 0.001 |
| CHST2 | 0.0445 | 0.0765 | −0.0035 | −0.2455 | −0.1565 | −0.1865 | −0.0445 | −0.1455 | 0.0595 | −0.0585 |
| CHURC1 | 0.78 | 0.689 | 0.768 | 0.495 | 0.408 | −0.054 | 0.883 | 0.75 | 0.082 | 0.992 |
| CIR | −0.0205 | −0.2215 | −0.1315 | −0.0455 | −0.0525 | 0.0865 | −0.2825 | 0.1815 | 0.5155 | −0.6125 |
| CIRBP | 0.1555 | 0.9585 | 0.4505 | 0.2315 | 0.0995 | −0.2525 | 0.3305 | 0.0935 | 0.2595 | −0.0835 |
| CKLF | −0.1405 | 0.3875 | −0.4355 | 0.8745 | 0.6485 | 1.2585 | 0.3805 | 1.8345 | −1.2015 | −1.2815 |
| CKLFSF6 | −1.0765 | 0.6415 | −0.3975 | −0.5275 | −0.7165 | 0.8955 | 0.3775 | 0.0875 | 1.3935 | 0.6835 |
| CKLFSF7 | −0.886 | −1.097 | −0.499 | −0.478 | −0.542 | −0.791 | 0.267 | −0.482 | 0.587 | −0.352 |
| CKMT1 | −1.627 | −0.942 | 0.122 | −1.352 | −0.944 | 0.104 | −0.207 | −0.421 | −0.365 | −0.564 |
| CKS1B | −0.146 | 0.077 | −0.261 | −0.124 | −0.451 | 0.193 | −0.185 | −0.4 | 0.653 | −0.263 |
| CKS2 | −0.5235 | 0.3475 | −0.5285 | −0.9985 | 0.8395 | −0.5135 | 0.7255 | −0.4755 | 0.5455 | 0.3015 |
| CL640 | −0.4885 | −1.3365 | −0.9145 | −0.2555 | 0.1205 | 0.6535 | −0.8335 | −0.0765 | −1.2005 | −1.3155 |
| CLDN23 | −0.7505 | −0.9105 | −0.7545 | −0.8065 | −0.4815 | −0.0255 | −0.2775 | −0.6475 | −1.2895 | −1.8825 |
| CLDN3 | −0.3265 | 0.0935 | 0.0435 | 1.4585 | 1.6975 | 1.0005 | 0.5755 | −0.2335 | −0.0195 | −1.0335 |
| CLDN4 | 0.535 | −0.044 | −0.084 | 0.185 | −0.424 | −1.506 | 1.143 | 2.004 | 2.096 | 0.319 |
| CLDN8 | 2.0815 | −0.3465 | 0.8155 | 0.1475 | 1.7025 | −0.0835 | 1.5885 | 2.2315 | 1.7945 | −0.8285 |
| CLGN | −0.2255 | 0.0765 | 0.0595 | −0.2725 | −0.1325 | −0.3965 | −0.5785 | 0.1315 | −1.2395 | −0.0655 |
| CLIC6 | 0.0689 | −0.085 | −0.139 | −0.175 | −0.686 | −0.379 | 0.004 | −0.781 | 0.405 | 0.413 |
| CLNS1A | 0.8805 | −0.1185 | 0.2045 | 1.5305 | 0.2715 | 1.5195 | 0.4415 | −0.1105 | 1.5955 | −0.8035 |
| CLTC | −0.174 | −0.384 | −0.029 | −0.134 | −0.498 | −0.481 | −0.081 | 0.142 | 0.284 | 0.164 |
| CLU | −0.12 | −0.373 | −0.855 | −0.34 | −0.48 | −0.632 | −1.083 | −0.22 | −0.472 | −0.59 |
| CMAS | −0.0375 | −0.2605 | −0.5245 | −0.0885 | −0.3985 | −0.8425 | −1.2795 | −0.0145 | 0.1945 | 0.3645 |
| CML65 | −0.395 | −0.118 | 0.068 | −0.56 | −0.075 | −0.001 | 0.319 | −0.054 | −0.028 | −0.177 |
| CNIH | −0.001 | 0.131 | 0.151 | −0.184 | 0.137 | 0.148 | 0.296 | 0.031 | −0.496 | −0.164 |
| CNOT10 | −0.0395 | 0.1785 | 0.4865 | 0.2145 | 0.1495 | −0.3785 | 0.2925 | −0.5725 | 0.2725 | 0.6565 |
| CNOT2 | 0.124 | −0.225 | −0.224 | 0.188 | −0.067 | 0.169 | −0.151 | −0.226 | 0.335 | 0.409 |
| CNOT7 | −0.333 | −2.779 | 1.316 | −0.249 | 3.907 | −1.727 | −1.181 | 1.647 | −0.249 | −2.042 |
| CNP | 0.3335 | 0.0755 | 0.1205 | 0.2985 | 0.2675 | 1.8225 | 0.7045 | 0.7905 | 1.0575 | 0.5145 |
| CNTNAP2 | −0.173 | −0.134 | −0.017 | 0.05 | 0.2 | −0.2 | 0.289 | −0.511 | −0.381 | −0.619 |
| COASY | −0.5375 | 1.3045 | −0.6725 | −0.0535 | 0.2115 | 0.8285 | 0.6265 | 0.3695 | −0.7035 | −1.2725 |
| COL11A2 | 1.066 | 2.012 | 1.244 | 0.959 | 1.051 | 1.18 | 1.025 | 0.293 | 0.46 | 0.184 |
| COL27A1 | 0.252 | −0.041 | 0.081 | 0.564 | 0.21 | −0.004 | 0.407 | −0.045 | −0.212 | 0.38 |
| COL4A5 | 0.3145 | 0.2715 | −0.1275 | 0.3205 | −0.1525 | 0.0395 | −0.7335 | 0.4195 | 1.2655 | −0.6595 |
| COL8A1 | 0.108 | −0.358 | 0.348 | −0.085 | −0.365 | −0.49 | −0.187 | 0.071 | 0.471 | −0.127 |
| COL8A3 | 0.052 | −0.456 | −0.246 | −0.454 | −0.343 | −0.084 | −0.253 | −0.014 | −0.247 | −0.128 |
| COMMD1 [symbol] | 0.0435 | −0.0155 | −0.0075 | 0.0905 | −0.3815 | −0.1555 | 0.1735 | −0.0715 | 0.3405 | −0.0145 |
| COMMD5 | 0.5565 | −0.0825 | 2.2465 | 0.3015 | −0.1845 | 3.3715 | 2.6535 | 2.9285 | 0.3485 | 0.3485 |
| COPS7B | −0.256 | −0.388 | −0.178 | −0.128 | −0.475 | −0.401 | 0.006 | 0.977 | −0.103 | −0.17 |
| CORO6 | −0.2915 | −0.2895 | −0.3605 | −0.8175 | −0.9025 | 0.1325 | −0.3765 | −0.0995 | 0.8825 | 0.2165 |
| CORT | −0.344 | −0.276 | −0.083 | −0.581 | −0.052 | −0.624 | −0.38 | −0.515 | 0.522 | −0.237 |
| COX17 | −0.294 | −0.288 | −0.226 | −0.428 | −0.023 | −0.572 | −0.347 | −0.612 | 0.59 | −0.163 |
| COX5A | 0.494 | −0.322 | −0.246 | −0.479 | −0.281 | 0.26 | −0.327 | −0.465 | 0.78 | 0.133 |
| COX6A | 3.1465 | −0.0825 | 2.2535 | −1.2945 | −1.0565 | 3.7005 | 0.8885 | −0.4095 | −4.4565 | 2.6675 |
| COX8A1 | −0.4575 | −0.1925 | −0.2025 | −0.5385 | −0.3345 | 0.2305 | −0.1255 | −0.0945 | 0.8575 | 0.2745 |
| COX8C | −1.1045 | −0.3055 | −0.8335 | 1.8115 | 0.2375 | 0.5355 | 0.0595 | 5.5725 | 5.2275 | −1.3185 |
| COX7A2 | −0.741 | −0.535 | −0.36 | 0.445 | 0.735 | 0.647 | 0.52 | 0.003 | −0.003 | −1.021 |
| CP | 3.0765 | 3.4275 | 2.2275 | 0.6495 | 0.7415 | −0.3785 | 0.7575 | 3.5535 | −4.0015 | 3.5015 |
| CPAMD0 | −0.306 | 1.007 | 0.457 | 0.129 | 0.511 | 0.42 | 0.839 | −0.525 | 0.229 | 0.267 |
| CPB1 | −0.1255 | 0.2735 | −0.2915 | −0.2095 | −0.5675 | 1.6605 | −0.5445 | 0.7335 | 1.1205 | 0.1125 |
| CPEB3 | −0.499 | −0.109 | −0.25 | −0.364 | 0.028 | −0.198 | 0.061 | 0.072 | −0.118 | −0.226 |
| CPEB4 | −0.05 | −0.231 | −0.49 | 1.072 | 0.671 | 0.286 | 0.235 | −0.485 | 0.933 | −0.207 |
| CPSF3 | −0.0055 | −0.8385 | −0.5715 | −0.3245 | 0.0985 | −0.2735 | 1.3965 | 0.4035 | 1.0685 | 0.0385 |
| CRABP1 | 1.093 | −0.252 | 0.758 | −1.473 | −1.128 | 0.844 | 0.232 | 0.676 | 0.717 | 0.553 |
| CREB3L4 | −0.025 | −0.213 | −0.216 | 0.053 | −0.291 | 0.693 | −0.224 | 0.07 | 1.2 | 0.814 |
| CRIP1 | 0.43 | 0.412 | 0.393 | 0.276 | 0.29 | −0.452 | 0.003 | 0.426 | 0.347 | −0.305 |
| CRYBB2 | 0.524 | −0.103 | 0.156 | 0.036 | −0.16 | −0.712 | −0.165 | 0.243 | 0.501 | −0.109 |
| CRYL1 | −0.104 | −0.113 | 0.017 | 0.121 | 0.85 | 0.338 | 0.081 | 0.51 | −1.651 | −1.381 |
| CRYZ | −0.3485 | −0.4325 | 0.1605 | −0.5155 | −0.1995 | −0.2505 | −0.2635 | −0.6385 | −0.2885 | −0.1605 |
| CSDA | −0.0195 | −0.1865 | −0.2595 | −0.0835 | −0.2515 | 0.0125 | −0.0465 | 0.0305 | −0.0315 | −0.1895 |
| CSE1L | 0.5655 | −0.2195 | −0.3745 | 1.3705 | 1.3835 | 0.0325 | 0.1275 | −0.7555 | 0.8875 | −0.0015 |
| CSK | 0.0035 | 0.0625 | 0.0575 | 0.1085 | 0.0855 | −0.0035 | 0.1925 | −0.2825 | −1.8015 | −0.8925 |
| CSRP1 | −0.1965 | 0.1775 | 0.0185 | 0.1095 | 0.7235 | −0.6165 | −0.3855 | 0.9225 | 1.9025 | 1.1655 |
| CSRP2 | 0.332 | −0.217 | −0.704 | −0.577 | 0.02 | 0.008 | −0.153 | −0.319 | 0.343 | −0.149 |
| CST5 | −0.6055 | −1.1075 | 0.6305 | 0.8585 | −0.3035 | −1.0845 | −0.9255 | −0.1375 | −0.6475 | −0.1345 |
| CSTB | −0.134 | −0.29 | −0.31 | −0.094 | 0.218 | −0.201 | 0.329 | 0.557 | −0.151 | 0.189 |
| CTAG1B | −0.3965 | −0.1875 | −0.0455 | −0.1955 | 0.1405 | 0.6275 | 0.0675 | −0.2205 | −0.2895 | −0.2045 |
| CTBP2 | 0.521 | 0.259 | −0.047 | 0.566 | 0.656 | −0.038 | 0.485 | 0.427 | −0.868 | −0.647 |
| CTNNA1 | −0.849 | −1.839 | 0.490 | 0.702 | 0.238 | −0.097 | 2.312 | −0.078 | −0.242 | −0.433 |
| CTNNAL1 | −0.7545 | 0.0025 | −0.0125 | −1.1765 | −0.3525 | −0.5815 | 0.1935 | 0.0525 | −0.0005 | −0.4655 |
| CTNND2 | −0.545 | −1.121 | −1.188 | 0.573 | −0.799 | −0.642 | −0.317 | −1.348 | −0.524 | −0.252 |
| CTPS | 0.791 | −0.04 | −0.079 | −0.658 | −0.89 | −0.739 | −0.411 | −0.881 | 1.989 | −0.069 |
| CTSL2 | −0.3565 | −0.2575 | −0.2125 | −0.8305 | −0.5415 | 0.1695 | −0.8615 | −0.6495 | 0.9335 | −0.6225 |
| CTSS | 0.423 | −0.13 | −0.6 | 1.086 | 1.36 | 0.89 | 0.95 | 1.228 | −1.82 | −1.293 |
| CTTN | −1.3385 | −1.4365 | 0.0425 | −2.5105 | −0.9235 | −1.5485 | 0.1165 | −0.0935 | −1.9505 | −1.5335 |
| CX3CL1 | −0.3715 | −0.7345 | 0.0845 | −0.3025 | −0.4225 | 0.4385 | −0.5295 | 0.2385 | 0.2955 | −0.2635 |
| CXCL9 | −0.0225 | −1.0465 | 1.9195 | 0.3545 | 0.3595 | 0.8135 | 1.9665 | −1.1765 | 0.1395 | −0.8605 |
| CXorf39 | 0.514 | −0.183 | 1.309 | 0.206 | −0.492 | 0.009 | −0.484 | −0.132 | 1.291 | 0.097 |
| CXXC4 | 0.107 | −0.345 | −0.149 | −0.161 | 0.15 | −0.398 | −1.13 | 0.021 | 0.408 | 0.672 |
| CYB5 | −0.2445 | −0.6295 | −0.2265 | −0.3935 | −0.1105 | −0.3715 | −0.3255 | −0.5385 | 0.1575 | −0.2715 |
| CYB561 | 0.378 | 0.028 | −0.199 | −0.114 | 0.533 | 0.59 | 0.546 | 0.291 | −0.238 | 0.344 |
| CYC1 | −0.105 | 2.358 | −0.301 | −0.05 | −0.205 | −0.08 | −0.197 | 3.733 | 0.027 | 1.18 |
| CYHR1 | −0.6905 | 4.0135 | 2.5405 | 1.9055 | −0.9005 | −0.3785 | −0.4135 | −0.0015 | 3.0905 | −0.6455 |
| CYP2A13 | −0.259 | −0.056 | 0.168 | −0.263 | 0.061 | −0.031 | 0.175 | 0.216 | 0.131 | −0.078 |
| CYP4X1 | 0.0895 | 1.8935 | 0.2465 | 0.7115 | 1.0235 | 1.5505 | 1.4375 | 0.2545 | 2.7815 | 1.1005 |
| D15Wsu7 [symbol] | −0.2085 | 0.7535 | 0.5835 | 1.2525 | 0.1125 | 0.2225 | 0.3215 | 1.4425 | 1.4575 | 0.9325 |
| DACH1 | 0.474 | 1.626 | −0.137 | 1.107 | 0.802 | 0.893 | 0.977 | 1.002 | 2.704 | 1.519 |
| DACH1 | −0.073 | −0.494 | −0.125 | −0.703 | −0.505 | −0.507 | 0.301 | −0.358 | 0.027 | 0.258 |
| DACH1 | 0.431 | −0.627 | 0.024 | −0.711 | −1.36 | −0.592 | −0.923 | 0.327 | 0.408 | 0.228 |
| DAP3 | 0.3245 | 0.1355 | 0.4055 | 0.2125 | −0.4735 | −0.3425 | −0.2795 | 0.2915 | 0.0325 | 0.0155 |
| DAPP1 | −0.864 | −0.86 | −0.198 | 0.928 | 0.769 | −0.416 | −0.128 | 0.942 | 1.478 | −1.02 |
| DAZAP2 | −0.339 | 0.356 | −0.307 | −0.375 | −0.108 | −0.313 | −0.195 | −0.473 | −0.585 | −0.016 |
| DBI | −0.6395 | −0.3275 | −0.3785 | −0.2785 | −0.2955 | −0.5495 | −0.6105 | 0.0145 | 0.0085 | −0.6885 |
| D9N1 | −0.583 | 0.07 | 0.065 | −0.536 | 0.125 | 0.085 | −0.376 | −0.291 | −0.812 | −0.396 |
| DC13 | −0.2815 | 0.1035 | 0.1155 | −0.3245 | −0.0355 | −0.2175 | −0.1845 | −0.0285 | −0.0495 | −0.0285 |
| DCPS | 0.6355 | 0.2275 | −0.0325 | 0.5255 | 0.2055 | 0.0505 | −0.0775 | 0.0725 | 0.0415 | 0.0795 |
| DDHD2 | −0.4925 | −0.5015 | −0.3265 | −0.7105 | −0.4875 | 0.2515 | 0.0065 | 0.1805 | −0.1195 | −0.8165 |
| D0X17 | 0.4485 | −0.1955 | −0.2685 | −0.4415 | 0.0915 | −0.4185 | 0.6825 | 1.2575 | −0.3755 | −0.0815 |
| DDX39 | −0.783 | −0.42 | −0.006 | −1.203 | −1.554 | −1.52 | −1.412 | 0.305 | −2.096 | −1.039 |
| DDX48 | −0.0915 | −0.1315 | 0.2495 | −0.3175 | −0.1545 | 0.1695 | 0.4385 | −0.0695 | −1.0205 | −0.6395 |
| DDX58 | −0.0435 | −0.1425 | −0.5525 | 0.2775 | −0.2865 | −0.5535 | 0.0235 | −0.2625 | 0.3395 | −0.4115 |
| DEF6 | −0.563 | −0.977 | −0.332 | 0.421 | 0.108 | −0.648 | −1.473 | 0.3 | 0.333 | 0.181 |
| DEK | 0.648 | 0.227 | 0.119 | 0.167 | −0.153 | −0.549 | −0.2 | 0.168 | −0.29 | 0.322 |
| DHCR7 | −0.65 | −0.262 | −0.296 | 0.206 | −0.006 | 0.216 | −0.007 | −0.773 | 0.181 | −0.589 |
| DHRS7 | −0.0615 | 0.7725 | −0.0725 | 0.5885 | 0.8935 | −0.3035 | 0.1055 | −0.2695 | −0.4295 | 0.3535 |
| DHX57 | 1.4095 | −0.0815 | −0.0345 | 2.9185 | 3.0305 | 2.5395 | 1.5485 | 3.4635 | 2.6155 | 0.1135 |
| DIRC1 | 0.098 | 0.18 | −0.35 | 0.489 | 0.249 | 0.873 | 0.047 | 1.277 | 0.48 | 0.157 |
| dJ222E13 | −0.036 | −0.265 | −0.27 | −0.333 | 0.023 | −0.393 | 0.028 | −0.489 | −0.084 | 0.575 |
| DKFZP434 | −0.587 | −0.471 | −0.833 | −0.587 | 0.237 | 0.063 | −0.198 | −1.048 | −1.41 | −1.054 |
| DKFZp434 | −0.5595 | −0.5635 | −0.1385 | −0.6235 | −0.3785 | −0.4455 | 0.1345 | −0.5615 | −0.2955 | −0.3085 |
| DKFZP434 | 0.049 | −0.025 | −0.875 | 0.229 | 0.376 | 0.358 | −0.247 | 0.189 | 0.006 | −0.061 |
| DKFZp434 | 0.2405 | −0.1115 | −0.1885 | −0.0785 | −0.4095 | −0.0645 | 0.1015 | 0.3195 | −0.3815 | 0.1905 |
| DKFZP564 | −0.3175 | −0.4065 | −0.2765 | −0.0485 | −0.1195 | −0.1365 | 0.0935 | 0.5605 | 0.0025 | 0.0595 |
| DKFZp564 | 0.141 | 0.167 | −0.132 | −0.715 | −0.716 | −0.651 | −0.276 | 0.468 | 0.06 | −0.428 |
| DKFZP564 | 0.1025 | 0.2235 | 0.0015 | 0.0415 | −0.0075 | −0.3595 | −0.1125 | 0.2485 | 0.7055 | 0.3095 |
| DKFZP564 | −0.012 | −0.271 | 0.472 | 0.152 | −0.328 | −0.728 | −0.891 | −0.481 | −0.276 | 0.142 |
| DKFZP564 | −1.151 | 0.436 | 0.459 | 0.386 | 0.902 | 0.307 | −0.109 | 1.814 | −1.596 | 0.098 |
| DKFZp586 | 0.1955 | 0.2585 | −0.2355 | 1.2555 | 3.8355 | 0.3955 | 2.0025 | 0.6085 | −0.8265 | 0.2665 |
| DKFZp761 | −0.021 | −0.032 | −0.012 | −0.052 | 0.073 | 0.045 | −0.028 | 0.281 | −0.203 | −0.073 |
| DLK1 | −0.062 | −0.176 | −0.459 | −0.368 | 0.11 | −1.014 | 0.188 | 0.162 | −0.162 | 0.011 |
| DLX5 | −0.3475 | 0.0575 | −0.2635 | −0.5935 | 0.0085 | 0.1725 | 1.1885 | 1.0705 | 1.2055 | −0.3415 |
| DNAJA4 | 0.1845 | 3.1385 | 1.5665 | 0.0435 | 0.2575 | 1.4055 | 1.8545 | 2.7345 | 3.0915 | 3.2475 |
| DNAJC1 | −0.061 | −0.539 | 0.124 | −0.576 | 0.881 | 0.105 | −1.1 | 0.32 | 0.393 | −1.027 |
| DNAJC12 | 0.07 | 1.102 | −0.788 | 0.503 | −0.534 | −0.019 | −1.491 | 1.231 | −3.77 | 0.875 |
| DNAJD1 | −0.362 | 0.003 | −0.149 | −0.24 | −0.25 | −0.439 | 0.093 | −0.205 | −0.363 | −0.357 |
| DNALI1 | 0.081 | −1.24 | 0.002 | −0.2 | −1.193 | −1.378 | −0.264 | −0.312 | −1.045 | −0.143 |
| DNMT1 | 0.4405 | 0.8275 | −0.8915 | 0.9755 | 1.2935 | 0.5085 | −0.0145 | −1.0705 | −0.8295 | −1.8525 |
| DOX1 | 0.1335 | 0.1945 | −0.3575 | −0.1755 | −0.5125 | −1.3545 | −0.4345 | 0.1925 | 0.5035 | 0.6615 |
| DSCR1 | −0.8575 | −0.9205 | 0.1905 | 0.2935 | 1.1465 | −0.8045 | 0.7425 | −0.2545 | −2.6865 | 0.7995 |
| DSCR1L2 | −0.4875 | −0.5215 | −0.3095 | −0.5305 | −0.3115 | −0.6485 | 0.1135 | −0.3345 | 0.1325 | −0.1975 |
| DSP | −0.2025 | −0.1655 | −0.3125 | 0.1945 | −0.1555 | 0.2535 | 0.4535 | −0.2265 | 0.6995 | 0.4635 |
| DTYMK | −0.0225 | −0.0025 | 0.6425 | −0.1425 | 0.8095 | −0.2485 | −0.0385 | 0.5165 | 0.8555 | 0.8805 |
| DUSP23 | 0.3905 | 0.8995 | 0.0205 | 1.0145 | 1.9845 | 0.0825 | 0.5395 | −0.0115 | −1.2335 | −1.0075 |
| DUSP4 | 0.0495 | −0.0055 | 0.1155 | −0.0615 | −0.0495 | −0.1555 | 0.2835 | 0.0705 | 0.1875 | 0.3545 |
| DUSP5 | −0.106 | −0.207 | −0.017 | −0.01 | −0.022 | −0.18 | −0.298 | −0.055 | 0.033 | −0.195 |
| DVL1 | 0.13 | 0.296 | −0.286 | 0.114 | 0.075 | 0.461 | 0.365 | −0.010 | −0.421 | −0.337 |
| DXYS155 [symbol] | −0.388 | −0.373 | −0.462 | −0.634 | −0.823 | −0.221 | −0.721 | 0.277 | −0.579 | −0.685 |
| DYRK4 | −0.3345 | −1.2225 | 0.1685 | 0.3735 | 0.5975 | 0.4435 | 0.4425 | −0.4815 | −0.6455 | −1.5505 |
| o(y)2 | 0.189 | −0.037 | 0.027 | −0.071 | 0.961 | 0.057 | 0.185 | −0.126 | 0.357 | 0.087 |
| EAF2 | 0.5665 | 0.0085 | −0.0425 | 0.9965 | 0.1785 | −0.0255 | 0.0745 | 0.1195 | −2.1845 | −0.2045 |
| EAP30 | 0.4435 | −0.1375 | −0.2815 | 0.3085 | −0.5415 | −0.0905 | 0.1565 | 0.3245 | 0.0205 | −0.4315 |
| ECHDC1 | −0.8595 | 0.6115 | 0.2165 | −0.7735 | −0.9035 | 0.8785 | −0.0585 | 2.0875 | 1.7835 | 1.1575 |
| ECHDC3 | 0.8055 | 1.4625 | 0.3635 | 0.8225 | 0.4375 | 1.3725 | 0.8775 | 0.0875 | 0.5445 | 1.7155 |
| EEF1A2 | −0.766 | −1.25 | −0.375 | −0.44 | −0.003 | −0.245 | 0.604 | −1.165 | 0.097 | −0.528 |
| EFHC1 | −0.2785 | 0.1105 | −1.2505 | 0.2075 | 0.9145 | −0.3455 | −0.5375 | 0.2535 | −0.4055 | 0.2925 |
| EFNA4 | 0.0555 | −0.7135 | −0.3815 | 0.1605 | −0.1785 | −0.3005 | −0.5255 | −0.8725 | −0.4975 | −0.2085 |
| EFS | 0.3235 | 0.1715 | −0.4775 | 0.1655 | −0.0255 | −0.3545 | 0.2465 | −0.4085 | 0.3015 | 0.1795 |
| EIF2C2 | −0.8905 | −0.0955 | −0.1125 | −0.5125 | −0.1955 | −0.6505 | −0.5025 | 0.7665 | −0.3835 | −0.2725 |
| EIF2C4 | −0.278 | −0.085 | −0.071 | −0.416 | 0.054 | 0.289 | −0.068 | −0.004 | −0.154 | −0.41 |
| EIF4EBP1 | −0.1555 | 0.6805 | 0.1355 | −0.4145 | 0.2015 | 0.4405 | 0.2795 | 0.3015 | 0.2775 | 0.9255 |
| EIF4G1 | 0.809 | 0.399 | 0.036 | 0.458 | −0.115 | −0.45 | −0.563 | −0.23 | −0.293 | 0.222 |
| EIF4G3 | −0.001 | 0.485 | −0.346 | −0.31 | −0.516 | 0.197 | −1.111 | 1.152 | 1.452 | 1.037 |
| ELAC1 | −0.429 | −0.96 | −0.29 | −0.353 | 0.606 | 0.473 | −0.339 | −0.921 | 0.73 | −0.196 |
| ELL2 | 0.3285 | 1.4085 | 2.2425 | 0.9545 | 1.0135 | 1.2435 | 0.0315 | 2.2945 | 1.7325 | 0.1115 |
| ELL3 | 0.0205 | −0.1915 | −0.1705 | −0.1335 | −0.4235 | −0.3065 | −0.5195 | −0.1785 | 0.0245 | 0.0005 |
| ELOVL5 | 0.5945 | −0.0705 | −0.4205 | −0.1975 | −0.2555 | −0.4325 | −0.5015 | −1.4115 | −2.6775 | −0.1765 |
| EN1 | 0.2705 | 0.8815 | 0.8245 | 0.8215 | 0.1135 | 0.1955 | 0.7455 | −0.3145 | 1.7205 | 0.6715 |
| ENAH | 0.5725 | 1.1045 | 0.2815 | 0.7485 | 0.9785 | 0.4825 | 0.1535 | −0.8975 | −0.0035 | −0.0515 |
| EPHX2 | 0.031 | −0.811 | −1.079 | −0.818 | 2.116 | −0.66 | 0.763 | −0.835 | 1.034 | 0.416 |
| EPLIN | −0.6755 | 1.0935 | 0.8555 | 0.0255 | 0.3875 | 0.3715 | 1.0705 | −0.6335 | −0.8045 | 1.0675 |
| EPN3 | 0.5005 | −0.3345 | −0.5325 | −0.3685 | 2.4525 | 1.1845 | 0.5755 | 0.0715 | −0.1115 | −0.3505 |
| EPOR | −0.233 | −0.56 | −0.009 | −0.302 | −0.372 | −0.445 | −0.56 | −0.452 | −0.532 | −0.327 |
| ERBB2 | 0.3315 | 3.3835 | 1.4005 | 1.8975 | −1.3175 | 0.3515 | 1.2905 | 1.3725 | 3.1825 | 2.8785 |
| ERP70 | −0.526 | 0.027 | −0.196 | 0.469 | 0.884 | −0.07 | −0.899 | −0.653 | −0.442 | −1.728 |
| ESR1 | −0.6335 | −0.2205 | −0.3705 | −0.2835 | 0.0315 | 0.1675 | 0.3975 | 0.5276 | −0.7285 | −0.2785 |
| ETFA | 0.4505 | −0.5925 | −0.1875 | −0.5085 | 0.2175 | −0.3475 | −0.0955 | −0.2455 | 0.7785 | −0.1555 |
| ETV8 | −1.0035 | −1.4075 | −0.5655 | −0.7135 | −1.2915 | −1.1425 | −0.0845 | −0.4335 | −1.0485 | −0.3135 |
| EVER1 | −0.3365 | −0.0825 | −0.5645 | −0.7095 | −0.1435 | −0.1395 | −0.0095 | 0.3455 | −0.1595 | −0.4925 |
| EXO1 | 0.2855 | 0.3405 | 0.1755 | 0.0055 | 0.3065 | 0.0675 | −0.4635 | −0.0055 | −0.7225 | −0.7045 |
| EXOSC9 | −0.833 | −1.073 | −0.087 | −0.77 | −1.268 | −1.319 | 0.061 | 0.258 | −0.16 | 0.197 |
| EXT2 | 0.025 | −0.22 | −0.429 | 0.004 | 0.276 | 0.152 | 0.478 | −0.905 | 1.408 | 1.029 |
| EZH2 | −0.0585 | 0.0535 | 0.0865 | −0.0275 | 0.0625 | −0.0105 | −0.1675 | −0.2535 | 0.2755 | 0.1525 |
| FI1R | −0.662 | −0.764 | −0.595 | 0.57 | 0.799 | 0.979 | −0.487 | 0.981 | 1.175 | −0.18 |
| F9 | −0.182 | 0.025 | 1.283 | 2.905 | 5.983 | −0.491 | −0.014 | −0.404 | −1.135 | −0.087 |
| FA2H | −0.259 | −0.205 | −0.308 | −0.339 | −0.076 | 0.261 | −0.711 | −0.192 | 1.986 | −0.14 |
| FABP7 | 0.223 | −0.402 | −0.426 | −0.408 | −0.082 | −0.425 | −0.245 | 0.315 | −0.149 | 0.012 |
| FADD | −0.425 | −0.106 | 0.165 | 0.075 | 0.258 | 0.388 | 0.113 | 0.23 | 0.052 | −0.319 |
| FAHD1 | −0.171 | −0.23 | 0.215 | −0.585 | −0.731 | −0.563 | 0.214 | −0.324 | 0.943 | 0.683 |
| FAHD2A | −0.316 | −0.324 | 0.018 | −0.186 | −0.221 | −0.007 | −0.206 | 0.271 | −0.011 | −0.233 |
| FAM38A | −0.1935 | −0.8795 | 2.4665 | 0.3205 | 3.9455 | 0.9225 | 1.0345 | −0.9695 | −1.1605 | 0.0005 |
| FAM3A | 0.3895 | 0.0445 | 0.3305 | −0.0875 | −0.2725 | −0.4715 | −0.9775 | −0.5795 | −0.1015 | 0.2025 |
| FAM3B | −0.107 | −0.487 | −0.128 | −0.108 | 0.191 | 1.621 | −0.264 | −0.409 | −0.329 | 0.156 |
| FAM3C | 0.032 | −0.512 | −0.059 | −0.778 | −1.387 | −1.262 | −1.25 | −1.145 | 0.098 | −0.182 |
| FAM46C | −1.089 | −1.145 | −0.694 | −1.81 | −1.029 | −1.351 | −0.164 | −0.807 | 0.008 | −0.257 |
| FAM49B | −1.123 | −1.306 | −0.967 | −1.033 | −0.837 | −0.776 | −0.212 | −0.043 | −0.038 | −0.743 |
| FAM54A | −0.533 | −0.399 | −0.129 | −0.227 | −0.108 | −0.078 | 0.003 | −1.021 | −1.198 | −0.299 |
| FANCA | −0.4215 | 0.0315 | 0.1955 | −0.1975 | 0.3515 | −0.0355 | 0.3075 | −0.2305 | −0.4885 | −0.8245 |
| FANCE | −0.3605 | 0.3425 | 0.2215 | −0.0655 | 0.4805 | 1.9045 | 1.0205 | 0.1875 | 1.2435 | 0.0295 |
| FBL | −0.178 | −0.134 | 0.032 | −0.272 | −0.073 | 0.125 | 0.39 | 0.087 | −0.539 | 0.227 |
| FBP1 | 0.125 | −0.398 | 0.094 | −0.615 | 0.561 | 0.288 | −0.807 | −0.681 | −0.067 | 0.207 |
| FBS1 | −0.0005 | 0.2055 | 0.5175 | 0.2755 | 0.0485 | −0.1585 | 0.1445 | 0.7895 | 0.1065 | −0.0535 |
| FBXL20 | 1.788 | −0.043 | −0.179 | −0.126 | 0.751 | 0.037 | 1.119 | 0.482 | 0.932 | 0.68 |
| FBXO25 | 0.399 | −0.323 | −0.326 | −0.463 | −0.062 | −0.227 | −0.456 | −0.142 | −0.152 | −0.144 |
| FDXR | −0.355 | −0.624 | 0.028 | 0.495 | 1.882 | 2.957 | 0.765 | 0.31 | 6.528 | 0.007 |
| FEM1B | −0.853 | 0.148 | −0.268 | 0.917 | 0.375 | −0.877 | 1.023 | 0.365 | 0.034 | −0.136 |
| FGF12 | −0.3085 | 0.1685 | −0.2295 | −0.0205 | 0.3215 | 0.7025 | 0.3845 | 1.0505 | 0.5085 | 0.1435 |
| FGF13 | −0.391 | 0.586 | 0.247 | −0.431 | −0.219 | −0.22 | 0.168 | −0.368 | 0.603 | −0.578 |
| FHOD1 | 1.1055 | 1.5815 | 0.2115 | 0.0155 | −0.0445 | 0.2015 | −0.0515 | 2.4245 | 0.2825 | 0.0545 |
| FKBP11 | −0.1905 | 0.2065 | 0.7085 | 0.8775 | 0.6505 | −0.1755 | −0.4185 | 1.2395 | 0.9495 | 0.9955 |
| FLJ10094 | 0.369 | 0.413 | 0.574 | 0.9 | 1.476 | 0.409 | 0.731 | −0.859 | −0.952 | −0.117 |
| FLJ10115 | 0.0915 | −0.4065 | −0.0475 | −0.8875 | −0.0475 | −0.1255 | −0.0875 | 0.1385 | −0.2825 | −0.2095 |
| FLJ10159 | 0.059 | 0.334 | 0.002 | 0.027 | 0.277 | 0.317 | 0.978 | 0.51 | −0.008 | 0.485 |
| FLJ10204 | −0.632 | −0.799 | −0.097 | −0.584 | −0.582 | −0.341 | −0.671 | −0.002 | 0.164 | −0.405 |
| FLJ10275 | 0.8585 | 1.5195 | 0.1195 | 0.9555 | 1.0965 | 1.0135 | 0.5935 | −0.0955 | −0.8785 | 1.5825 |
| FLJ10292 | 0.0295 | −0.1395 | −0.4095 | 0.0655 | −0.4285 | −0.3185 | −0.1335 | 0.6195 | 0.6025 | 0.6135 |
| FLJ10324 | −0.3025 | −0.7945 | −0.4545 | −0.5965 | −0.8165 | −0.7975 | 0.1835 | −0.6395 | 0.1835 | 0.1895 |
| FLJ10700 | −0.138 | 0.251 | 0.246 | −0.079 | 0.169 | 0.146 | 0.6 | −0.539 | 0.461 | 0 |
| FLJ10706 | −0.429 | −1.832 | −0.357 | −1.005 | 0.47 | −1.365 | −0.643 | −1.826 | −0.908 | −0.608 |
| FLJ10671 | 0.013 | −0.503 | −0.168 | 0.423 | 0.338 | −0.07 | −0.12 | 1.374 | 0.692 | −0.24 |
| FLJ10901 | −0.1505 | 0.5525 | 0.1925 | 0.5825 | 0.2805 | 1.6575 | 0.8065 | 1.0885 | 1.1265 | 1.3745 |
| FLJ10918 | −0.564 | −0.858 | −0.766 | 0.335 | 0.208 | 0.364 | −0.048 | −0.594 | −0.71 | −0.731 |
| FLJ10980 | 0.5115 | −0.2385 | 0.9885 | 0.3315 | −0.2315 | −0.2415 | −0.3575 | 0.0285 | 1.0305 | 4.6585 |
| FLJ11017 | −0.0975 | −0.3465 | 0.3485 | −0.6865 | −0.3815 | −0.3445 | −0.4965 | −0.9825 | −0.4755 | 0.0205 |
| FLJ11088 | 0.3645 | −0.1035 | 0.0925 | 0.4755 | −0.1535 | 0.0945 | 0.1825 | 0.5355 | −0.1485 | 0.1065 |
| FLJ11151 | 0.3265 | −0.2365 | 0.2825 | −0.4085 | −0.2145 | −0.1795 | 0.6095 | −0.0755 | 1.3195 | 1.1915 |
| FLJ11267 | −0.0735 | 0.2215 | −0.1955 | −0.0335 | −0.1825 | 1.6505 | 0.2055 | 0.4195 | −0.0785 | −0.1715 |
| FLJ11280 | −0.1385 | 0.5285 | −0.3415 | 0.2115 | 0.5885 | 1.0375 | 0.5835 | 0.6135 | −0.0095 | −0.3615 |
| FLJ11508 | 0.1505 | −0.0335 | 0.9765 | 0.7225 | −0.4355 | −0.3385 | −0.3655 | −0.6995 | −1.1015 | −0.0765 |
| FLJ12270 | 1.4095 | 1.3795 | 0.4765 | −0.1825 | −0.6965 | 1.5285 | 1.7435 | 1.5495 | 3.7975 | 2.7995 |
| FLJ12844 | 0.0885 | −0.1215 | −0.4845 | −0.2875 | 0.0465 | 0.1055 | −0.0495 | −0.0035 | −0.1905 | −0.0485 |
| FLJ12650 | −0.1965 | −0.6245 | −0.0715 | −0.3375 | −0.5485 | −0.0255 | −0.0145 | −0.1365 | −0.1075 | 0.2105 |
| FLJ12684 | −0.5595 | −0.4375 | −0.3175 | 0.0085 | 0.3745 | 0.1715 | 0.0705 | −0.6665 | −0.1485 | −0.9495 |
| FLJ12735 | −0.64 | 0.708 | 0.55 | 0.469 | 0.214 | 1.1 | 0.453 | 0.607 | −2.804 | 1.194 |
| FLJ12750 | 0.888 | 0.951 | −0.271 | 0.51 | 0.965 | 0.904 | 0.782 | 1.034 | 0.139 | 1.991 |
| FLJ12895 | 0.3785 | −0.3295 | −0.5285 | −0.6325 | 0.5485 | 0.0395 | 0.7295 | −0.2275 | −0.4005 | 0.3365 |
| FLJ13710 | −1.0715 | −0.2985 | 0.1145 | −0.1465 | −0.7545 | −0.3845 | −0.2526 | 0.6705 | −0.3385 | −0.3045 |
| FLJ13855 | 0.005 | −0.096 | −0.232 | 0.016 | −0.216 | −0.51 | 0.877 | 0.424 | −0.091 | 0.217 |
| FLJ13912 | −0.231 | −0.985 | −0.338 | −1.373 | −0.731 | −0.724 | −0.139 | 0.715 | 0.073 | −0.003 |
| FLJ14124 | −0.604 | −0.491 | 0.211 | 0.01 | 0.111 | −0.235 | 0.001 | −0.53 | −0.83 | −0.359 |
| FLJ14627 | 0.1585 | 0.0125 | −0.0695 | 0.0705 | 0.0785 | 0.0315 | −0.0315 | −0.0495 | −0.0875 | 0.1376 |
| FLJ14666 | 0.003 | −8.305 | 0.046 | −0.693 | −0.443 | −0.633 | −0.37 | 0.205 | −1.143 | −0.326 |
| FLJ14904 | 1.441 | 1.597 | −0.133 | 8.268 | −0.376 | −0.139 | 1.097 | 1.452 | 1.46 | 2.204 |
| FLJ20035 | 0.082 | −0.393 | −0.881 | −0.528 | −0.281 | −0.283 | −0.325 | −0.311 | −0.563 | −0.57 |
| FLJ20152 | −0.431 | −0.176 | −0.251 | −0.228 | 0.122 | 8.058 | 0.276 | 0.093 | −0.349 | −0.81 |
| FLJ20171 | 0.094 | 0.094 | 0.099 | 0.187 | 0.455 | 0.262 | 0.527 | −0.34 | 0.546 | −0.086 |
| FLJ20244 | 0.209 | −0.593 | −0.003 | 0.245 | 0.565 | 0.674 | 1.224 | 0.208 | −3.055 | −1.888 |
| FLJ20273 | 0.084 | 0.176 | 0.068 | −0.079 | −0.64 | −0.302 | −0.233 | 0.024 | −0.428 | −0.345 |
| FLJ20315 | −0.362 | −0.839 | −0.348 | −0.01 | −1.195 | −1.349 | −0.866 | 0.262 | 0.559 | 0.021 |
| FLJ20323 | 0.1995 | −0.4495 | −0.1245 | −0.3535 | −0.2505 | −0.4205 | −0.5625 | −0.9565 | −0.3455 | −0.2275 |
| FLJ20518 | −0.413 | 0.589 | 8.375 | −0.239 | −0.13 | 0.011 | 0.504 | 0.916 | −0.895 | −0.6 |
| FLJ20530 | −0.364 | 0.023 | 0.003 | −0.228 | −0.337 | −0.502 | −0.475 | 0.598 | 0.052 | −0.407 |
| FLJ20696 | −0.0635 | −0.2905 | −0.1875 | −0.6185 | −0.3835 | −0.4795 | −0.5195 | −8.3425 | −0.6065 | −0.4385 |
| FLJ20718 | −0.215 | −0.149 | −0.424 | −0.27 | 0.345 | 0.295 | 0.356 | 0.226 | −0.203 | −0.163 |
| FLJ20772 | 0.2045 | 0.0045 | −0.0425 | 0.1475 | 0.0095 | 1.3355 | 0.9055 | 0.4865 | −0.1165 | 0.2175 |
| FLJ20989 | 1.1775 | 1.2225 | 0.7685 | 0.8015 | 1.2465 | 1.1415 | 0.9655 | −0.7015 | −8.7715 | 0.7185 |
| FLJ21019 | −0.5395 | 0.1455 | −0.7405 | 0.3895 | 0.8055 | 0.4145 | 0.1025 | −0.0075 | −2.3425 | 0.8095 |
| FLJ21062 | −0.8715 | 0.4195 | −1.6515 | 0.9045 | −0.0465 | 0.8785 | 0.1575 | −1.9615 | 0.5165 | 1.0705 |
| FLJ21159 | 0.5095 | 0.8125 | 1.2005 | 0.6735 | 0.6195 | 0.1065 | 8.4015 | 0.3685 | −0.2625 | 1.1145 |
| FLJ21616 | 0.135 | 0.372 | 0.886 | 1.006 | 1.501 | 0.313 | −0.737 | 1.143 | −0.541 | 1.048 |
| FLJ21827 | −0.0365 | −0.3825 | 0.4795 | 0.0895 | 0.5035 | −0.6345 | −0.9445 | −0.7215 | −0.8905 | −0.4275 |
| FLJ21963 | 0.806 | −0.312 | −0.415 | −8.168 | 0.055 | −0.586 | 0.259 | 0.203 | −0.389 | 0.15 |
| FLJ22104 | 0.0635 | −0.3635 | −0.0495 | −0.8205 | −0.1345 | −0.0715 | −0.3675 | −0.6105 | −0.5185 | −0.7485 |
| FLJ22222 | −0.075 | −0.149 | 0.313 | 0.254 | 0.215 | −0.004 | 0.341 | −0.864 | −1.377 | −0.084 |
| FLJ22573 | 0.3395 | −0.1805 | 0.0485 | −0.0055 | −0.0025 | 1.1905 | 1.0225 | 8.3355 | −0.9745 | −0.3475 |
| FLJ22794 | 0.312 | 0.311 | 0.032 | −0.042 | −0.051 | −0.575 | −0.129 | −0.202 | −0.304 | 0.175 |
| FLJ23188 | 0.4175 | 1.4195 | 0.5575 | −0.3005 | 0.0915 | 0.0565 | −0.0055 | 5.0135 | 0.0155 | 0.2105 |
| FLJ23441 | 0.073 | 0.34 | 0.26 | 0.154 | −0.079 | −0.287 | 0.049 | 0.362 | 0.59 | 0.386 |
| FLJ25471 | −0.05 | 0.159 | −0.27 | −0.418 | −0.179 | 0.183 | −0.3 | 0.05 | 0.732 | 0.25 |
| FLJ31204 | 0.2515 | −0.2795 | 0.4035 | 0.2965 | −0.1245 | −0.0525 | −0.1045 | 0.0015 | −0.6915 | 0.4725 |
| FLJ31795 | −0.0125 | 0.1375 | −0.1165 | −0.3645 | −0.0015 | 0.4375 | −0.0595 | 0.5015 | −0.9635 | −0.2745 |
| FLJ32942 | 0.6685 | 0.6075 | 0.0065 | 0.8235 | 0.2525 | −0.1735 | −0.3875 | −0.9085 | 1.6666 | 2.2695 |
| FLJ37970 | 0.468 | −0.886 | −0.632 | 0.353 | 0.274 | 2.831 | 0.279 | 0.799 | 0.27 | 0.488 |
| FLJ39370 | 0.5335 | −0.0955 | −0.2105 | −0.2685 | 0.3735 | −0.7685 | 0.5255 | 0.0365 | −0.0075 | −0.0125 |
| FLJ90588 | 0.3345 | −0.2745 | 0.1735 | 1.2875 | 0.3575 | 0.0955 | 0.0205 | −0.1715 | −1.6785 | 0.1275 |
| FN3KRP | 0.064 | 1.148 | −0.002 | −0.096 | −0.091 | −1.178 | 0.73 | 0.804 | 1.394 | 1.274 |
| FOLR1 | 0.2565 | 0.1805 | −0.3395 | 0.9195 | 0.6705 | 0.3705 | 0.3005 | −0.8425 | −0.2385 | −1.0115 |
| FOXA1 | 0.2185 | 0.4495 | 0.1785 | 0.3575 | 0.4235 | 0.3145 | 0.4155 | −0.0185 | 0.6215 | 0.5295 |
| FOXC1 | −0.428 | 0.446 | −0.188 | −0.828 | −1.105 | 0.322 | −0.082 | −0.664 | −0.593 | −0.834 |
| FOXP1 | 0.7555 | 1.3375 | 1.2155 | 1.2155 | 1.7885 | 0.6845 | 0.4865 | 1.0295 | 0.8825 | 0.7605 |
| FREQ | −0.4165 | −0.0325 | −0.6455 | −0.2995 | −0.5305 | −0.4425 | −0.5855 | −0.3305 | 0.0795 | −0.0135 |
| FTO | 0.6145 | 0.4475 | 0.4505 | 0.0315 | −0.7495 | −0.7135 | −0.1835 | −0.6755 | −0.4735 | 0.2885 |
| FUBP1 | 0.4785 | 0.3125 | 0.2085 | −0.4995 | −0.0135 | 1.1085 | 1.4605 | 0.8505 | 2.0305 | −0.0855 |
| FUCA1 | 0.368 | −0.024 | 0.037 | 0.291 | 1.107 | 0.876 | 0.637 | −0.953 | −1.745 | −0.575 |
| FUT8 | 0.2375 | 0.0065 | −0.1645 | 0.1675 | 0.7446 | −0.0255 | 0.0425 | −0.4255 | 0.5765 | 0.0345 |
| FXYD6 | −0.242 | 0.343 | 0.976 | 0.014 | 0.487 | 1.013 | 0.427 | 1.082 | −1.482 | 1.194 |
| FYCO1 | 1.046 | −0.043 | −0.123 | 1.285 | 1.371 | 0.793 | 0.262 | −0.419 | 0.299 | −0.296 |
| FZD10 | −0.1016 | −8.4625 | 0.2445 | −0.0275 | −0.8915 | −0.6035 | −0.5125 | 0.0675 | −0.6245 | −0.2105 |
| FZD7 | −0.4325 | −1.3145 | −0.2135 | −1.9225 | −1.8615 | −0.9445 | −1.5255 | 1.4235 | −2.1215 | −0.5945 |
| FZD9 | −0.0596 | −0.9455 | −0.0255 | −0.9055 | −1.1915 | −0.6875 | −1.1065 | 2.1825 | −1.3575 | 0.3345 |
| G1P2 | −0.2165 | 0.1255 | −0.3735 | −0.0145 | −0.0605 | −0.5885 | −0.7995 | 0.0915 | 0.1455 | −0.2335 |
| G1P3 | 0.126 | 0.512 | 0.04 | 0.559 | −0.037 | 0.35 | −0.418 | 0.418 | 0.836 | 0.012 |
| GA17 | −1.9185 | −2.1365 | −1.2785 | −1.7145 | −0.7335 | −1.1495 | −0.3425 | −0.2805 | 0.8865 | −0.6485 |
| GABARAP | 0.0985 | −0.5715 | 0.1895 | −0.5875 | −0.3985 | 0.1755 | −0.4525 | 0.0985 | 0.8375 | 0.3895 |
| GAJ | −0.1035 | 0.8595 | −0.6575 | 0.4005 | −0.0225 | 1.7525 | −0.2405 | 2.0105 | 2.1595 | 2.3355 |
| GALE | 0.219 | −0.103 | −0.151 | −0.992 | −0.378 | −0.33 | 0.015 | −0.405 | 1.643 | 0.129 |
| GALNT14 | 0.8975 | 0.3725 | −0.0345 | 0.3075 | 0.4455 | −0.3385 | −0.5855 | −0.9045 | 1.8585 | 0.6585 |
| GALNT2 | −0.0905 | 0.0065 | −0.0095 | −0.0465 | −0.0405 | 0.1465 | 0.7825 | 0.7865 | 1.5845 | 0.9005 |
| GALNT7 | −0.408 | −0.265 | −0.376 | −0.721 | −0.076 | −0.044 | −0.157 | −0.296 | 0.108 | −0.109 |
| GAMT | −0.783 | 0.05 | −0.665 | −1.065 | −0.243 | −0.172 | −0.159 | −0.001 | −0.681 | −0.957 |
| GAPD | −0.399 | 0.059 | −0.324 | −0.62 | 0.038 | 0.098 | 0.262 | −0.29 | −0.302 | −0.198 |
| GARS | 0.004 | 1.226 | 1.107 | −0.257 | −0.746 | 0.321 | 0.735 | 1.942 | 8.096 | 1.049 |
| GART | 1.1395 | 0.9395 | −0.4845 | 1.1515 | −0.8885 | −0.6135 | −0.3975 | 1.0995 | 0.5295 | 0.9285 |
| GATA3 | −0.0435 | −0.3175 | −0.7045 | −0.5916 | −0.3925 | −0.0605 | 0.2975 | −0.0785 | 0.4835 | 0.9225 |
| GATM | 2.288 | 2.626 | −0.686 | −0.026 | −0.157 | 1.895 | 0.311 | 1.371 | 1.542 | 1.541 |
| GBA | −0.077 | 0.53 | 0.158 | −0.556 | 0.233 | −0.515 | −0.136 | −0.709 | −0.2 | −0.141 |
| GDAP1 | 0.066 | 0.467 | 0.013 | −0.188 | 0.342 | 0.685 | 0.85 | 0.252 | −0.469 | 0.213 |
| GDI2 | −0.3995 | 0.5165 | 0.2545 | 0.2435 | −0.1185 | 0.1025 | 0.4115 | 0.0545 | 0.3765 | 1.0165 |
| GGA1 | −0.0045 | 0.3075 | 0.7525 | −0.0005 | −0.1625 | 0.7315 | 0.7515 | 0.0785 | −0.2485 | −0.7605 |
| GKAP1 | 0.3085 | 0.1055 | 0.2015 | −0.0555 | 0.5685 | 0.3575 | −0.0105 | 0.1475 | −0.2985 | −0.4395 |
| GLCCI1 | −0.1355 | 0.4835 | 0.8765 | 0.3405 | 0.7785 | 0.2185 | 0.6295 | 0.3585 | −0.1125 | −0.5675 |
| GLG1 | 0.401 | −0.121 | 0.213 | −0.26 | −0.975 | −0.648 | −0.791 | −1.23 | 0.52 | −0.3 |
| GLTSCR2 | 0.392 | −0.634 | 0.653 | 0.055 | −0.772 | −0.407 | 0.245 | 0.208 | 0.559 | 0.316 |
| GNB4 | −0.191 | −0.049 | −0.172 | 0.108 | −0.125 | −0.038 | 0.161 | −0.118 | 0.286 | −0.429 |
| GOLPH2 | −0.3705 | −0.1355 | 0.0555 | −0.0715 | −0.0205 | 0.2235 | 0.3235 | 0.0405 | −0.7715 | −0.1225 |
| GPATC1 | −0.3185 | −0.0035 | −0.3585 | 0.7715 | 0.4345 | −0.0245 | 0.3055 | −0.3735 | −0.6475 | −0.1995 |
| GPC2 | 0.9525 | 0.0405 | 1.0335 | 0.0045 | 0.0315 | −1.2875 | 0.2125 | −0.3385 | 0.7245 | 0.7475 |
| GPR125 | −0.634 | −0.654 | −1.426 | −0.037 | 0.471 | 0.357 | −0.632 | 0.214 | −0.468 | −1.219 |
| GPR160 | −0.0095 | 0.3315 | −0.3805 | 0.1345 | 0.8765 | 0.0265 | 0.4935 | 0.5915 | 0.5755 | 0.7135 |
| GPR56 | −0.4385 | 0.8265 | −0.1855 | 0.1925 | 0.4775 | 0.8725 | 0.8485 | −0.0145 | −1.2305 | 0.1445 |
| GPRC5C | −0.8885 | −0.1205 | −0.4305 | −0.8085 | −0.3685 | −0.0385 | 0.0066 | −0.3375 | −0.0265 | 0.7645 |
| GPSM1 | 0 | 0.787 | −0.097 | 0.857 | 0.686 | −0.238 | 0 | −0.382 | −0.048 | 0.347 |
| GPSM2 | −1.756 | −0.226 | −1.439 | 0.36 | 0.005 | −0.61 | −1.023 | 2.255 | 1.418 | −0.419 |
| GPT | −0.0665 | 0.0735 | 0.4095 | −0.2825 | −0.3005 | 0.1705 | −0.3735 | 0.1965 | −0.7935 | −1.1425 |
| GPT2 | −0.933 | 0.024 | 2.225 | 0.308 | 3.245 | −0.074 | −0.657 | −1.619 | 4.492 | 6.394 |
| GPX7 | −0.098 | −0.159 | −0.296 | −0.077 | 0.58 | 0.187 | −0.071 | 0.246 | −0.115 | 0.003 |
| GRB14 | −0.1405 | 0.4235 | 0.2795 | 0.3365 | 0.6665 | 0.8615 | −0.2825 | −0.9855 | 1.8565 | 1.4695 |
| GRB7 | −0.3285 | −0.0635 | −0.1715 | 0.0835 | 0.4395 | 0.7265 | 0.4095 | 0.2855 | −0.4725 | −0.2605 |
| GSC | 0.049 | −0.125 | −0.214 | 0.026 | 0.047 | −0.071 | 0.159 | −0.047 | 0.319 | 0.117 |
| GSDML | −0.167 | −0.404 | 0.052 | −0.492 | −0.21 | −0.272 | −0.11 | −0.006 | −0.282 | −0.198 |
| GSK3B | 0.283 | 0.187 | 0.261 | 0.384 | 0.541 | 0.885 | 0.241 | −0.046 | 0.653 | 0.084 |
| GSTA1 | 0.7015 | −0.0085 | 0.8515 | 0.2245 | 1.2205 | −0.8705 | 0.8425 | 2.7135 | 2.2285 | 0.4895 |
| GSTK1 | −0.505 | 0.095 | −0.801 | 0.262 | 0.011 | −0.128 | 0.837 | −0.863 | −2.528 | −1.613 |
| GSTM3 | 0.4305 | −2.6405 | −0.1485 | 0.0885 | −2.4705 | −0.7995 | 0.9855 | 0.2705 | 1.1485 | 0.6205 |
| GSTP1 | 0.807 | 1.548 | −0.077 | 2.985 | 0.898 | 0.245 | −0.085 | 0.732 | −0.502 | 0.423 |
| GSTT1 | −0.0595 | −0.0675 | 0.2115 | −0.1025 | −0.0705 | 0.0585 | −0.2185 | 0.2535 | −0.7195 | −1.0755 |
| GSTT2 | −0.0025 | 0.1495 | −0.2285 | 0.0115 | −0.0735 | 0.2365 | 0.1385 | −0.2945 | −0.0405 | 0.2105 |
| GTF2F2 | −0.148 | 0.207 | 0.327 | −0.463 | −0.108 | 0.472 | 0.017 | 0.311 | −0.018 | −0.775 |
| GTF2I | −0.3405 | −0.1715 | −0.6505 | −0.1825 | −0.2515 | 1.0775 | 0.5235 | 0.0755 | 0.2085 | −0.3055 |
| GTF3A | −0.413 | −0.106 | −0.235 | −0.726 | −0.03 | −0.286 | −0.253 | −0.304 | −0.327 | −0.644 |
| GTPBP3 | −0.152 | 0.264 | −1.529 | −0.922 | 0.153 | 0.408 | 1.645 | −0.253 | −0.419 | 0.345 |
| GTPBP4 | −0.288 | −0.8 | −0.208 | −0.809 | −0.829 | −0.603 | −0.195 | −0.403 | −0.146 | −0.082 |
| H1F0 | 0.0205 | −0.1175 | −0.0925 | −0.6515 | 0.2845 | 0.2465 | 0.8995 | 0.3095 | 0.1685 | 0.9125 |
| H2AFV | −1.0825 | −0.4995 | −0.6495 | −1.2565 | −0.4315 | −0.9255 | −0.0135 | 0.0385 | 0.1675 | −0.1785 |
| H2AFY | 0.3785 | −1.5405 | −0.5495 | −0.8345 | −1.1025 | −0.5955 | −1.3725 | −0.1515 | 0.5285 | −0.0325 |
| H2AFZ | 0.8425 | 0.0385 | −0.5805 | −0.4955 | 0.3685 | −0.3165 | 0.7605 | −0.4945 | −0.7365 | 0.2015 |
| H2BFS | −0.7785 | −0.3065 | 0.8155 | −0.7385 | −0.6205 | −0.1345 | 0.3585 | −0.3925 | −0.7045 | −0.8945 |
| H3F3B | 0.104 | 0.083 | −0.187 | −0.432 | −0.457 | −0.047 | 0.328 | 0.195 | 0.263 | 1.188 |
| H41 | −0.0705 | 0.5565 | 0.2755 | 0.1825 | 0.3105 | 0.6105 | 0.4805 | 0.4175 | −0.3755 | 0.1885 |
| HAGH | 0.162 | −0.342 | −0.089 | −0.045 | −0.076 | −0.074 | −0.169 | 0.909 | −0.464 | 0.053 |
| HBG1 | −1.4135 | −0.8475 | −1.3275 | −1.4335 | −0.5175 | −0.5165 | 0.1765 | −0.8615 | −0.6045 | −0.2505 |
| HBL01 | −0.411 | −0.002 | −0.011 | −0.466 | −0.283 | −0.651 | −0.333 | 1.459 | −0.342 | 0.313 |
| HCAP-G | −0.4895 | 0.0705 | −0.0115 | −0.0235 | −0.0045 | −0.6715 | 0.0665 | −0.4525 | −0.1725 | −0.0395 |
| HCP5 | 1.234 | 0.951 | 0.534 | 0.403 | 0.622 | 1.835 | 0.398 | −0.057 | −0.694 | −0.531 |
| HDAC2 | 0.862 | 0.134 | 0.566 | 0.113 | −0.386 | 2.419 | 1.057 | 0.552 | 0.971 | −0.628 |
| HDC | 0.0435 | −0.1845 | −0.1225 | −0.0145 | −0.1535 | −0.0635 | −0.0725 | 0.3815 | 0.0965 | 0.0575 |
| HEBP1 | 0.312 | 1.258 | −0.17 | −0.18 | 1.51 | 0.026 | 0.867 | 0.871 | −0.499 | −1.663 |
| HIPK2 | 1.125 | −0.015 | 0.458 | 0.225 | −1 | 0.519 | −0.436 | 0.313 | −0.288 | 1.587 |
| HIPK2 | −0.0785 | −1.6685 | −1.3925 | −0.2565 | −0.5945 | −0.0035 | −0.3645 | −0.5385 | 1.2665 | 0.8535 |
| HIS1 | −0.0275 | −1.3205 | −1.1185 | −0.5575 | −0.4975 | 0.0055 | −0.8755 | −0.0055 | 0.7555 | 0.1145 |
| HIST1H2A | 0.034 | −1.272 | −1.041 | −0.815 | −0.442 | −0.021 | −0.888 | −0.025 | 0.73 | 0.151 |
| HIST1H2B | 0.0015 | −1.2005 | −0.9615 | −0.6425 | −0.2975 | −0.0015 | −0.7705 | 0.2595 | 0.4945 | 0.2395 |
| HIST1H2B | 0.176 | −1.128 | −1.114 | −0.804 | −0.269 | 0.131 | −0.73 | −0.063 | 0.714 | 0.241 |
| HIST1H2B | 0.7215 | −0.1565 | −0.4865 | −0.1235 | 0.1355 | −0.5185 | 0.4735 | −0.4895 | −0.2785 | 0.1475 |
| HIST1H2B | −0.3175 | −0.3335 | 0.0605 | −0.5995 | −0.0785 | 0.0015 | −0.4835 | −0.1825 | −0.3605 | −0.3935 |
| HIST1H3C | 0.0645 | −0.6205 | −0.0875 | −1.2055 | −0.1125 | −1.0065 | −0.4245 | 0.5985 | 0.7255 | 0.0145 |
| HM13 | −0.5095 | −0.8015 | −0.9025 | −1.5775 | −0.0295 | −1.1505 | −0.3385 | −0.0005 | −0.2945 | −0.4985 |
| HMGB3 | −0.2685 | −0.3455 | −0.1215 | −0.8025 | −0.2015 | −0.4685 | −0.2075 | −0.1735 | 0.1475 | −0.3475 |
| HN1 | 0.223 | −0.012 | −0.189 | −0.589 | −0.096 | −0.723 | −0.237 | 0.488 | 0.065 | −0.062 |
| HNRPA3P | 0.8825 | 0.0875 | 0.1335 | 2.1595 | 1.5425 | 1.2445 | 0.9805 | −0.4025 | −0.4405 | −0.1815 |
| HNRPDL | 0.974 | 0.402 | 0.793 | 2.577 | 1.877 | 1.59 | 1.46 | −0.541 | −0.711 | −0.607 |
| HOXA5 | 1.5565 | 1.3505 | 0.6935 | 0.1435 | 1.9635 | 0.9645 | 1.7415 | −2.6135 | 2.6495 | 1.7105 |
| HOXA7 | 2.4825 | −0.1135 | 0.6055 | −0.0556 | 1.2125 | −0.2275 | −0.4405 | −0.8515 | 0.9325 | 0.6535 |
| HOXB2 | 0.5105 | −0.2775 | 0.2605 | −1.0115 | 1.6835 | 0.2305 | 0.0065 | −0.5915 | −0.8855 | −0.0185 |
| HOXB6 | 0.0005 | −0.8445 | −0.1235 | −0.0495 | 0.6965 | 0.7295 | −0.3285 | −1.1885 | −1.0935 | −0.2045 |
| HOXB7 | −0.0815 | −1.3235 | 0.3355 | −0.2505 | −0.1825 | 0.3295 | −1.0595 | −1.2115 | −0.8635 | −0.2005 |
| HOXC10 | 0.373 | −1.019 | 0.1 | −0.298 | 0.11 | −0.26 | 0.072 | −2.233 | −1.858 | −0.081 |
| HOXC13 | −0.0935 | 0.8575 | 0.0455 | 0.4445 | 0.7205 | −0.4695 | 0.1845 | −0.1775 | −0.2295 | 0.0175 |
| HOXC9 | 0.088 | 0.245 | 0.495 | 0.356 | −0.248 | −0.171 | 0.241 | 0.069 | 0.417 | −0.22 |
| HOXD3 | −0.1815 | −0.0975 | −0.4765 | −0.1815 | −1.0425 | 0.1535 | −0.6685 | 0.5585 | 0.3125 | −0.1315 |
| HPCL2 | −1.15 | −1.094 | 1.051 | −0.495 | −0.256 | −1.307 | −0.894 | −1.782 | −2.041 | −0.768 |
| HPS3 | −0.2795 | 0.0025 | −0.1535 | −0.4155 | 0.4655 | −0.3145 | −0.6855 | −0.5565 | −0.4875 | −0.6605 |
| HRASLS | −0.3495 | 0.3575 | −0.8155 | −0.7815 | −0.9565 | −0.5265 | −1.1465 | 0.4925 | −0.2005 | −0.3235 |
| HRB | 0.045 | −1.595 | 0.017 | 0.888 | 0.842 | 0.28 | 0.202 | −0.219 | −1.529 | −0.241 |
| HRSP12 | 0.037 | −0.071 | −0.27 | −0.279 | −0.115 | 0.21 | −0.196 | −0.347 | −0.508 | −0.55 |
| HSD17B2 | −0.1635 | −0.4655 | −0.1785 | −0.3465 | −0.1545 | −0.1475 | −0.0355 | 0.0535 | −0.2745 | −0.4455 |
| HSF1 | −0.1445 | −0.2165 | −0.2955 | −0.1905 | 1.6935 | −0.3575 | 1.3105 | 1.5805 | −0.7925 | 0.0455 |
| HSPA14 | −0.849 | −1.457 | −1.547 | −0.523 | 1.156 | 0.654 | −0.51 | −0.811 | −1.331 | −0.776 |
| HSPA2 | −0.2515 | 0.0535 | −0.2045 | −0.5116 | 0.1885 | 0.4665 | 0.3055 | −0.1255 | 0.6685 | −0.2305 |
| HSPA5BP [symbol] | −0.054 | 0.126 | 0.38 | 0.125 | −0.17 | −0.431 | −0.516 | 0.113 | −0.367 | 0.081 |
| HSPC016 | −0.065 | −0.027 | 0.027 | −0.055 | 0.208 | −0.126 | −0.057 | 0.194 | −0.879 | −0.119 |
| HSPC138 | −1.72 | −1.225 | −1.115 | −1.52 | −1.421 | −1.539 | 0.101 | −0.867 | 0.909 | 0.278 |
| HSPC148 | −0.3865 | −0.8475 | −1.1835 | −0.8945 | 0.6135 | −0.5975 | −0.0895 | −0.5615 | 0.2945 | −0.2635 |
| HSPC150 | −0.2875 | 0.2185 | 0.0085 | 0.2775 | 0.4035 | −0.0445 | 0.0845 | −0.0105 | −0.1745 | 0.1635 |
| HSPC163 | −0.211 | 0.429 | −0.157 | −0.156 | −0.037 | −0.36 | −8.515 | 0.772 | −0.655 | −0.524 |
| HSPC266 | −0.004 | 0.037 | −0.806 | −0.387 | −0.961 | 0.141 | −1.095 | −0.312 | 1.485 | 0.351 |
| HSU79274 | −0.131 | −0.22 | −0.253 | −0.749 | −0.356 | −0.228 | −0.543 | −0.008 | 0.097 | −0.398 |
| HTATIP2 | −0.121 | −0.607 | −0.635 | −0.458 | 0.475 | −0.604 | 0.097 | −0.029 | 0.241 | 0.069 |
| ICMT | 0.987 | 0.113 | 0.345 | 1.539 | 0.562 | 0.496 | 0.848 | −0.94 | −1.277 | −1.572 |
| ICT1 | −0.711 | −0.914 | −0.422 | −0.322 | 0.554 | −0.866 | −0.756 | −0.555 | −0.891 | −1.156 |
| ID4 | 0.0105 | 0.2475 | 0.0835 | −0.0105 | −0.8255 | 0.2845 | 0.5105 | 8.5755 | 0.5685 | −0.2295 |
| IDH2 | 0.484 | −0.307 | −0.688 | −1.42 | −1.181 | −0.263 | −0.258 | −0.713 | −1.05 | −1.088 |
| IDS | 0.0285 | −0.4165 | −0.3595 | −0.6105 | −1.1005 | 0.2285 | −0.5885 | 0.4705 | −1.8705 | −0.5775 |
| IFI30 | −0.2625 | −0.1795 | −0.1845 | −0.2535 | −0.3515 | −0.5355 | −0.3805 | 0.0275 | −0.1625 | −0.1065 |
| IFI44 | 0.0855 | 0.0285 | −0.0125 | −0.3455 | −0.2885 | −0.0645 | −0.2015 | 1.7625 | −1.2895 | −0.5965 |
| IFIT5 | 0.0665 | 0.5485 | 0.3145 | 0.4235 | 0.3555 | −0.0465 | −0.0145 | 0.1205 | 0.5425 | 0.7815 |
| IFITM1 | 2.0235 | 0.8525 | 0.1115 | −0.3135 | 0.6065 | 0.5125 | 2.0705 | −0.0405 | 1.3135 | 1.0275 |
| IGBP1 | 0.3065 | −0.8005 | 1.4475 | −0.1585 | 0.7445 | 0.9935 | 0.7135 | −0.5015 | 1.8775 | 1.8545 |
| IGFALS | −0.2285 | −0.6605 | 0.1185 | −0.5355 | −0.0275 | −0.4185 | −0.0205 | −0.4885 | −0.5085 | −0.8065 |
| IGPBP2 | −0.1085 | −0.4605 | 0.0065 | −0.1235 | −0.2075 | −0.6935 | −0.1065 | −0.5545 | −0.4005 | −0.1695 |
| IGHG1 | 1.2075 | −1.4955 | 2.0245 | 1.0535 | 1.4715 | −0.7675 | 1.6045 | −2.3485 | −3.6805 | −2.8915 |
| IGHG1 | 0.144 | −1.556 | 1.288 | −0.281 | −0.297 | −1.291 | −0.656 | −0.934 | −1.377 | −1.544 |
| IGJ | 0.501 | −2.263 | 0.753 | −0.364 | −0.801 | −2.102 | −0.306 | −1.127 | −2.499 | −2.83 |
| IGKC | 0.15 | −0.794 | 0.134 | −0.174 | −0.27 | −0.787 | 0.097 | −0.514 | −0.384 | −0.692 |
| IGKC | 0.156 | −1.237 | 0.394 | −0.509 | 0.123 | −0.834 | 0.098 | −1.109 | −1.584 | −1.633 |
| IGLC2 | 0.4815 | 0.0425 | 0.1745 | −0.3135 | 0.1805 | −0.4275 | −0.7885 | 0.2095 | −0.2855 | −0.2765 |
| IGLL1 | 1.2015 | 0.4005 | 0.5435 | 1.8415 | 2.2465 | 1.2995 | 0.6835 | −0.2035 | −0.1845 | −0.0615 |
| IL13RA1 | −0.169 | −0.254 | 0.897 | −0.057 | −0.089 | −0.521 | −0.04 | 0.668 | −0.534 | −0.041 |
| IL17B | 0.3885 | 0.9425 | 0.4585 | 1.1305 | 0.5105 | 0.8875 | 0.3905 | 2.1175 | 0.5285 | 1.6805 |
| IL23A | −0.1275 | −0.5425 | −0.4155 | −0.2585 | −0.2675 | −0.8735 | 0.0745 | 0.1275 | 0.4245 | 0.2025 |
| IL6ST | −0.8455 | −0.3055 | −0.1115 | −0.4115 | −0.3045 | −0.0405 | 0.0065 | 0.8045 | 0.5385 | −0.5425 |
| ILF2 | −0.296 | −0.005 | −0.243 | 0.023 | −0.303 | −0.126 | 0.212 | 0.481 | 0.697 | 0.033 |
| IMPA2 | 0.3615 | 0.2815 | −1.2445 | 1.2235 | 0.8075 | 0.8945 | −0.1975 | −0.8785 | 0.4905 | −2.0835 |
| ING1L | −0.14 | 0.398 | −1.301 | 0.84 | 0.624 | 1.745 | 0.341 | 1.639 | −0.285 | 0.196 |
| INHBB | 0.0305 | 0.2945 | 0.1925 | −0.0165 | 0.5315 | −0.1585 | −0.0015 | −0.0895 | 6.9005 | 6.0135 |
| INPP4B | 0.9545 | −1.2585 | 0.0335 | 1.1435 | 1.1285 | 0.8115 | 0.2725 | −0.6975 | 0.8695 | 0.2225 |
| INSM1 | 0.058 | 0.086 | 0.342 | −0.141 | 0.132 | 0.203 | 0.337 | −0.728 | −0.643 | 0.161 |
| IQCA | 1.081 | 0.568 | 1.104 | 1.955 | 0.999 | 1.191 | 0.012 | 0.234 | −1.584 | −1.937 |
| IQCE | −0.223 | −0.536 | 0.435 | −0.054 | −0.414 | −0.398 | −0.049 | −0.095 | −1.408 | −0.534 |
| IQGAP2 | −0.4125 | −0.3776 | −0.1285 | −0.4505 | 0.0625 | −0.6125 | −0.1865 | −0.4095 | −1.0745 | −0.8705 |
| IRF4 | 1.3015 | 2.1815 | 0.3175 | 1.8425 | 1.9255 | 2.6325 | 1.2735 | 2.9315 | −1.5645 | −1.1885 |
| IRTA2 | −1.54 | −1.294 | −0.726 | −1.063 | −0.28 | −0.045 | −0.638 | 0.475 | −0.858 | −0.71 |
| IRX4 | −0.2155 | −0.3945 | −0.3615 | −0.7845 | −0.7775 | −0.4305 | −0.4565 | 1.4255 | −1.3835 | −0.1565 |
| ISG20 | −0.12 | 0.884 | −0.965 | 0.616 | 1.29 | 0.515 | 0.569 | −0.372 | −2.135 | −2.388 |
| ISGF3 [symbol] | 1.294 | 1.822 | 0.68 | 1.523 | 0.165 | 0.788 | 1.726 | 2.384 | 8.342 | 0.847 |
| ITGB4 | −0.141 | 0.107 | −0.027 | −0.018 | 0.388 | 0.291 | 0.629 | 0.112 | −0.293 | −0.046 |
| ITPR1 | 0.838 | −0.553 | 0.385 | −0.339 | −0.871 | 0.157 | 0.33 | −0.635 | 1.357 | 1.7 |
| JARID1A | −0.001 | −0.088 | 0.248 | 0.021 | −0.307 | −0.745 | −0.17 | −0.44 | −0.313 | −0.23 |
| KARCA1 | −0.124 | 1.556 | −0.019 | −0.177 | 0.139 | −0.004 | −0.156 | 0.105 | 0.673 | 3.362 |
| KATNA1 | 0.2215 | 2.8275 | −0.1595 | −0.2325 | 0.2245 | 0.2385 | −0.1475 | 2.7845 | −0.3405 | 0.3275 |
| KCNC2 | −0.259 | −0.478 | −0.08 | 0.022 | −0.013 | 0.111 | −0.055 | 0.489 | −0.222 | −0.203 |
| KCND2 | −0.252 | −0.438 | −0.221 | 0.956 | 0.748 | 1.314 | −0.003 | −0.813 | 0.695 | −0.377 |
| KCNK4 | −0.01 | 2.154 | 0.408 | 0.201 | −0.687 | −0.812 | −0.846 | 2.154 | 1.545 | 0.889 |
| KCNN4 | −0.2405 | −0.3875 | −0.1935 | 0.4805 | 0.6815 | 0.5295 | 0.2285 | 1.7705 | 1.3535 | −0.6455 |
| KCNS3 | 1.5175 | −0.1165 | 0.2195 | 0.0905 | 0.3485 | 0.6355 | 0.5245 | −0.3105 | 1.4495 | 1.0815 |
| KCTD15 | −0.31 | 0 | −0.034 | −1.758 | −0.1 | −0.606 | −0.398 | −0.136 | −0.29 | −0.523 |
| KCTD3 | −0.171 | −0.042 | 0.208 | −0.185 | −0.331 | −0.333 | −0.269 | 0.264 | −0.385 | −0.516 |
| KDELR2 | −1.195 | −1.252 | −1.057 | −1.808 | −0.761 | −0.321 | 0.057 | −0.645 | 0.72 | 0.172 |
| KIAA0020 | −0.124 | −0.436 | −0.229 | −0.224 | 0.415 | 0.431 | 0.463 | 0.351 | 0.282 | 0.102 |
| KIAA0101 | 0.3905 | 0.3615 | −0.3785 | 0.2175 | 0.7615 | 0.9235 | 0.7095 | 0.7475 | 0.4685 | −0.0025 |
| KIAA0153 | 0.8405 | 0.0205 | 0.0915 | −0.2785 | 0.0945 | −0.2295 | −0.2205 | −0.7285 | −0.2045 | −0.1585 |
| KIAA0182 | −0.3995 | −0.5685 | −0.0535 | −0.6205 | −0.2135 | 0.0835 | −0.3605 | −0.2985 | −0.7525 | −0.4555 |
| KIAA0196 | −0.1325 | −0.2835 | −0.0465 | −0.3545 | 0.1245 | −0.5825 | 0.6125 | −0.1695 | 0.0425 | −0.1035 |
| KIAA0241 | 0.03 | 0.223 | 0.524 | −0.176 | −0.042 | −0.535 | 0.133 | −0.425 | 0.353 | 0.031 |
| KIAA0251 | −0.3915 | 0.1195 | −0.0265 | 0.0225 | 0.1245 | 0.4195 | 0.1795 | 0.1435 | 0.0405 | −0.1775 |
| KIAA0274 | −0.217 | 0.561 | 0.212 | 0.734 | 0.733 | 0.657 | 1.047 | −0.075 | 0.087 | 0.58 |
| KIAA0515 | −0.4275 | 0.2015 | −0.0035 | −0.1515 | −0.0625 | −0.1255 | 0.1625 | −0.3515 | −0.8485 | 0.0515 |
| KIAA0582 | −0.8565 | −0.8015 | −0.4285 | 0.4555 | 0.0515 | 0.8675 | 1.4545 | 0.3818 | 0.0265 | −0.4355 |
| KIAA0650 | 0.1145 | 0.3675 | −0.1476 | 0.2125 | 0.5575 | −0.2795 | −0.1715 | −0.4945 | 0.7655 | 1.4745 |
| KIAA0703 | 0.948 | 0.799 | −0.3 | 0.064 | 0.119 | 0.174 | 0.383 | 0.343 | −0.849 | −0.054 |
| KIAA0830 | 0.6215 | 0.4465 | 0.1595 | 0.7035 | 1.1715 | 0.0185 | 0.0385 | 0.7885 | 1.7955 | 0.8585 |
| KIAA0980 | 0.3925 | −0.6275 | 0.1666 | 0.2205 | −0.2355 | −1.3975 | −1.1955 | −0.4955 | 0.6555 | 0.8095 |
| KIAA1324 | 0.357 | 0.698 | 1.484 | 0.696 | 0.911 | 0.732 | 1.059 | 1.505 | 0.835 | 0.119 |
| KIAA1363 | −0.272 | 1.302 | 1.405 | 0.878 | 0.138 | 0.298 | 1.101 | −0.055 | 0.465 | 1.341 |
| KIAA1683 | −0.4665 | −0.9035 | 0.2145 | −0.1855 | −0.7385 | −0.4275 | 0.2505 | −0.1905 | −0.9045 | 0.8325 |
| KIF12B | −0.2175 | −0.0275 | −0.1795 | 0.0405 | −0.0855 | 0.0015 | −0.0245 | −0.1875 | −0.0895 | −0.4195 |
| KIF24 | −0.0055 | −0.0245 | 0.1855 | 0.0545 | −0.0975 | −0.1925 | −0.1035 | 0.2115 | −0.0025 | 0.0895 |
| KIN | 0.3205 | −0.0005 | 0.8535 | 2.4715 | 1.8285 | 1.4045 | 1.1855 | −2.0675 | −0.8455 | −1.4725 |
| KIRREL3 | 0.9315 | 0.4015 | −0.2855 | 1.9215 | 1.3755 | 0.9985 | 0.8725 | −0.2105 | 0.2895 | −0.5375 |
| KIT | 1.0755 | −0.1585 | −0.2175 | 2.4635 | 2.0455 | 0.7455 | 0.7955 | −0.6855 | 0.3645 | −1.1495 |
| KLK5 | 0.572 | 1.136 | −0.056 | 2.214 | 2.368 | 1.618 | 0.53 | −0.498 | 1.103 | −0.514 |
| KLK6 | 0.529 | −0.261 | −0.474 | 2.255 | 1.382 | 1.275 | 1.05 | −0.645 | 0.126 | −0.294 |
| KLK7 | −1.78 | −0.97 | −0.771 | −1.882 | −0.742 | −0.787 | −0.342 | −1.091 | −0.459 | −0.3 |
| KLK8 | −0.4275 | −0.1205 | −0.8925 | −0.7215 | −0.2405 | −0.2615 | 0.3775 | −0.5135 | 0.1535 | −0.1105 |
| KNTC2 | 0 | −0.007 | 0 | −0.015 | −0.247 | −0.347 | 0.142 | 0.293 | 1.221 | 0.808 |
| KPNA2 | 0.5025 | −0.2775 | 0.0615 | −0.0475 | −0.5625 | 0.6545 | −0.2475 | 0.2445 | 0.3465 | 0.3085 |
| KREMEN2 | 1.0245 | 1.3955 | −0.5965 | 3.4355 | 1.5995 | 1.4465 | 1.2585 | −0.5345 | 0.1275 | −0.8465 |
| KRT10 | 0.8965 | 2.1115 | −0.8755 | 3.0885 | 2.3445 | 2.0945 | 1.9005 | −1.1005 | −0.3455 | −1.1155 |
| KRT13 | 1.667 | 0.613 | −0.602 | 1.545 | 2.137 | 1.49 | 1.142 | −1.615 | 0.074 | −1.535 |
| KRT15 | −0.424 | −0.047 | −0.315 | −0.775 | 0.514 | 1.58 | 0.457 | 0.864 | −0.529 | −0.441 |
| KRT17 | 0.0515 | 0.1005 | −1.8355 | 0.0745 | 0.0645 | −0.6945 | −0.6935 | −0.7135 | 3.1675 | 1.0055 |
| KRT18 | 1.118 | 0.759 | −0.102 | 2.126 | 1.659 | 1.415 | 0.765 | −2.852 | 1.015 | −1.448 |
| KRT23 | 0.0275 | 0.0545 | 0.0885 | −0.1005 | −0.1275 | 0.2525 | −0.0015 | 0.0985 | 1.5185 | 0.1375 |
| KRT6B | 0.382 | −0.084 | −0.092 | 0.27 | −0.422 | 0.487 | −0.175 | 0.178 | −0.222 | 0.195 |
| KRTAP3-3 | 1.3725 | 0.3815 | −0.0015 | 1.3505 | 2.4995 | 1.3795 | 1.8365 | 0.6945 | 1.6675 | 0.5665 |
| KRTHA7 | 1.2185 | 0.0645 | 0.0845 | 1.0245 | 2.1545 | 1.3145 | 1.3245 | −1.4965 | 1.3345 | −0.5345 |
| LAMA3 | 0.042 | −0.042 | −0.181 | −0.46 | −0.192 | 0.128 | 0.87 | 0.001 | 0.999 | 0.536 |
| LAMB3 | −0.251 | −0.384 | −0.093 | −0.197 | −0.237 | −0.426 | −0.322 | −0.182 | −0.504 | −0.383 |
| LASS2 | −0.0885 | −0.3285 | −0.1305 | −0.4295 | −0.4735 | −0.4655 | −0.0395 | −1.3905 | 0.4615 | 0.2505 |
| LAX | 0.0045 | 1.1135 | −0.0045 | 1.6115 | 0.7716 | −0.8885 | −0.5965 | −0.6985 | −1.4185 | 0.4125 |
| LBR | −0.4265 | 0.0415 | −0.4235 | 0.0345 | 0.5345 | −0.2665 | −0.0425 | −0.9125 | −2.4195 | −0.8795 |
| LCE1B | 0.1515 | 0.5465 | 0.3955 | 0.0185 | 0.1785 | −0.4425 | 0.2395 | −0.3505 | 0.2025 | 0.6325 |
| LDHB | 0.18 | 0.827 | 0.171 | 0.727 | 0.002 | 0.642 | 0.182 | −0.107 | −0.098 | 0.315 |
| LDOC1L | −0.792 | 0.582 | −1.031 | −0.158 | 0.463 | 0.135 | 0.882 | 1.218 | 0.374 | 0.017 |
| LETMD1 | 0.2065 | −0.2375 | 0.5745 | 0.1075 | 0.2215 | −0.6065 | 0.5415 | 0.2015 | −2.8135 | −1.5225 |
| LFNG | 0.13 | −0.141 | −0.178 | 0.202 | 0.075 | 0.184 | 0.396 | −0.163 | 1.394 | 0.248 |
| LGALS2 | −0.212 | 0.384 | −0.367 | −0.021 | 0.146 | −0.118 | −0.804 | −0.297 | 0.716 | 0.456 |
| LGTN | 0.182 | 1.731 | −0.415 | −0.117 | 0.125 | 1.453 | 3.345 | 0.897 | 0.836 | 2 |
| LNX | −0.4795 | −0.0165 | 0.2855 | −0.2185 | −0.2085 | −0.3215 | −0.2585 | 0.4475 | 0.6635 | −0.4415 |
| LOC11247 | 0.242 | −0.372 | 0.306 | −0.025 | −0.405 | −0.485 | 0.033 | −0.182 | −0.481 | −0.132 |
| LOC11492 | −0.172 | −0.106 | 0.721 | −0.184 | 0.029 | −0.219 | −0.116 | 0.353 | 1.104 | 0.481 |
| LOC11497 | −0.1485 | 0.1065 | 0.1635 | 0.2845 | −0.0635 | 0.0135 | 0.0885 | 1.6635 | 0.9335 | 0.0435 |
| LOC11621 | −0.8465 | 0.6405 | 0.3085 | 1.5105 | 2.1835 | 1.8625 | −0.1735 | 2.9395 | −0.8035 | −0.1305 |
| LOC11644 | 0.2695 | 0.3035 | 0.1875 | −0.2905 | −1.1285 | −1.9765 | 0.5115 | 0.9085 | 0.1945 | 0.5435 |
| LOC12022 | 0.0015 | −0.0675 | 0.0905 | −0.2265 | −0.1025 | 0.0635 | 0.3005 | 0.2835 | −0.2945 | 0.3515 |
| LOC12422 | 1.0075 | 0.2505 | 0.3965 | 0.1205 | 0.2835 | 1.2165 | 0.2365 | −0.4885 | 0.9385 | 0.8075 |
| LOC14654 | −0.311 | −0.339 | −0.071 | 0.075 | 0.551 | −0.804 | −0.133 | 0.238 | 0.447 | 0.058 |
| LOC15506 | −0.086 | 2.24 | −0.12 | 0.225 | −0.103 | −0.217 | −0.205 | 1.514 | 2.999 | 2.062 |
| LOC20189 | 1.2265 | 2.4285 | 0.0495 | 0.6535 | 1.7465 | 0.6865 | −0.1895 | −1.1085 | −0.8725 | −0.4555 |
| LOC25398 | 0.448 | 0.035 | 0.166 | 0.115 | 0.57 | 0.113 | −0.075 | 0.277 | −0.473 | −0.47 |
| LOC33990 | 0.185 | 0.335 | −0.388 | 0.113 | 0.581 | 0.204 | 0.099 | 1.475 | 0.167 | −0.202 |
| LOC34817 | −1.5895 | 0.4885 | 0.6655 | 0.1965 | 0.2835 | 2.4485 | 1.2215 | 0.9705 | −0.3615 | 0.3425 |
| LOC40045 | 0.46 | 0.422 | 0.564 | −0.42 | −0.985 | 0.118 | −0.685 | −0.67 | −1.322 | −0.24 |
| LOC44089 | 0.889 | −0.292 | −0.176 | −0.383 | −0.579 | 0.029 | −0.659 | −0.486 | −0.614 | −0.061 |
| LOC49386 | 0.903 | 0.643 | −0.404 | 0.024 | −0.099 | 0.08 | 1.036 | 0.081 | 0.42 | 1.173 |
| LOC51123 | 0.639 | −0.083 | −0.006 | −0.344 | 0.007 | −0.186 | 0.124 | −0.082 | 0.258 | 0.505 |
| LOC51161 | 0.2655 | −0.7875 | −0.5955 | 0.0365 | −0.8395 | 1.4675 | 1.4195 | 1.8825 | −1.1305 | 0.5805 |
| LOC51321 | 0.6555 | −0.0485 | −0.1295 | 0.4665 | 0.4505 | −0.4415 | 1.2115 | −0.8535 | −1.8965 | 0.6415 |
| LOC51760 | −0.1965 | −0.3295 | −0.6465 | −0.7175 | −0.1225 | 0.1665 | −0.1475 | −1.5115 | −0.2715 | 0.0485 |
| LOC81558 | 1.306 | 1.103 | 0.07 | −0.142 | 0.32 | 1.89 | 0.02 | 0.362 | 0.575 | 0.505 |
| LOC81569 | 0.4495 | −0.3335 | −0.0975 | 0.0395 | −0.0315 | −0.4395 | −0.5865 | −0.5275 | 1.5925 | 0.3155 |
| LOC90355 | −0.629 | −0.399 | −0.414 | 0.587 | 0.177 | −0.18 | −0.728 | 0.164 | 0.871 | −0.519 |
| LOC90701 | 1.34 | −1.649 | 0.799 | −1.205 | 0.282 | −1.848 | 0.515 | −1.308 | −2.528 | −1.501 |
| LONP | 0.2175 | 0.7465 | −0.0785 | −0.1735 | 0.1655 | 9.0285 | 0.4725 | 0.2815 | 0.7255 | 0.2065 |
| LRAP | 0.6415 | 0.5045 | 0.5425 | 0.8765 | 0.6805 | 0.4605 | 0.9065 | 1.0585 | 0.6115 | 0.4065 |
| LRBA | −0.3005 | 0.1985 | 0.0025 | 0.8275 | 0.5855 | −0.0615 | 0.4615 | 0.4215 | 0.0615 | −0.5115 |
| LRIG1 | −0.8925 | −0.8545 | −0.7975 | −0.7775 | −1.5505 | −1.0415 | −1.3545 | 0.0445 | 0.7085 | −0.1735 |
| LRP6 | 1.944 | 0.93 | 1.247 | 0.163 | 0.906 | 1.384 | 1.908 | −0.127 | −0.075 | 0.934 |
| LRP8 | 0.298 | −0.057 | 0.413 | 0.394 | 0.366 | −0.311 | 0.528 | −0.153 | 0.697 | −0.489 |
| LRRC17 | −0.4075 | −0.1195 | 0.2815 | −0.5055 | 0.0015 | −0.5345 | −0.0315 | 0.4065 | −0.1305 | −0.3825 |
| LRRC2 | −0.0655 | 0.0385 | −0.0595 | −0.1435 | −0.3635 | −0.2545 | −0.2135 | −0.0075 | −0.3655 | 0.2355 |
| LSM1 | −0.7255 | 0.0355 | 0.2365 | −0.3765 | 0.3055 | −1.1535 | −0.0975 | −0.0095 | −2.0305 | −2.6525 |
| LSM8 | 0.035 | −0.384 | −0.289 | −0.146 | 1.19 | −0.28 | −0.099 | 0.241 | 0.063 | −0.342 |
| LTB | −0.138 | 0.404 | −0.362 | 0.067 | 0.148 | 0.033 | −0.072 | −0.819 | −0.909 | −1.038 |
| LY6D | −1.289 | −0.825 | −0.855 | −1.583 | −1.165 | −1.685 | −0.375 | −0.921 | 0.205 | −0.499 |
| LYN | −0.025 | −0.389 | −0.096 | −0.204 | −0.337 | −0.13 | −0.043 | 0.107 | 0.298 | 0.3 |
| MA02L1 | 0.046 | −0.185 | −0.353 | −0.029 | −0.602 | 0.497 | 0.541 | 0.331 | −0.278 | 0.053 |
| MADP-1 | 0.1175 | −0.0105 | 3.1895 | 0.3135 | 0.0425 | 0.0125 | −0.1925 | 0.0775 | −0.7845 | 0.3425 |
| MAGEA12 | −0.06 | −0.279 | 0.081 | 0.15 | −0.065 | −0.189 | −0.144 | 0.31 | −0.263 | −0.03 |
| MAGEA2 | 0.4145 | −0.0735 | −0.0745 | 0.3085 | 0.0415 | −0.0075 | −0.0295 | −0.5205 | −0.7535 | 0.3835 |
| MAGEA3 | −0.0755 | 0.2385 | −0.1375 | −0.0075 | 0.2315 | 1.8815 | −0.2665 | −0.3805 | −1.2085 | −0.7005 |
| MAGEA4 | −0.011 | 0.187 | 0.065 | −0.4 | −0.295 | −0.295 | −0.464 | −0.286 | 2.472 | 4.634 |
| MAGED4 | 0.2085 | 0.4355 | −0.0785 | −0.0585 | 0.3635 | 0.9295 | 0.3485 | 1.9245 | 0.6345 | −0.3235 |
| MAGI1 | −0.0895 | −0.6935 | 0.0615 | −0.1655 | −0.3675 | −0.4585 | −0.4605 | −0.1505 | −0.7125 | −1.1995 |
| MAGI-3 | 0.085 | −0.227 | 0.021 | −0.347 | −0.045 | −0.113 | −0.312 | 0.049 | −0.201 | −0.708 |
| MAL2 | 0.239 | 1.288 | 1.18 | −0.999 | −1.158 | 0.983 | 0.175 | 0.247 | 1.691 | 1.267 |
| MAN2B1 | −0.8295 | 2.9655 | −0.8935 | −0.4175 | 0.6785 | −0.1235 | 1.2445 | −0.5175 | −0.0645 | −0.7955 |
| MAPK8IP2 | −0.8255 | 1.4155 | 0.2485 | −0.9105 | −1.4865 | −0.3865 | −1.2595 | −0.7475 | 3.1455 | 2.5145 |
| MARCO | 0.07 | 1.060 | 0.669 | 0.461 | 1.17 | −0.284 | 0.42 | 1.203 | 2.22 | 1.08 |
| MARLIN1 | −0.597 | −0.524 | −0.295 | −0.59 | −0.513 | −0.114 | 0.241 | 0.251 | −0.128 | −0.319 |
| MASS1 | −0.7525 | −0.4815 | −0.4195 | −0.8135 | −0.1605 | −0.2395 | 0.3925 | 0.1835 | −0.3455 | −0.3865 |
| MCM2 | 0.3055 | −1.1905 | −0.7315 | 0.8995 | −0.7425 | −1.2005 | 0.4825 | −0.2895 | −1.3045 | −1.0295 |
| MCM3 | 0.397 | −1.755 | 0.26 | −0.89 | −0.452 | 1.275 | 0.383 | −0.186 | 0.643 | 0.02 |
| MESP1 | 0.6295 | 0.0445 | 0.6685 | −1.2165 | −0.6445 | 0.7255 | 0.6265 | 0.0015 | −0.7985 | −0.0595 |
| METRN | 0.4505 | −0.1845 | 0.0075 | 0.3595 | 0.0435 | −0.3205 | −0.1115 | −0.2915 | −0.2665 | −0.2455 |
| MFAP2 | −0.525 | −0.079 | −0.308 | −0.292 | 0.176 | 0.02 | −0.05 | −0.846 | −0.126 | 0.001 |
| MFGE8 | −0.4095 | −0.4025 | −0.6275 | −0.5745 | −0.2295 | 0.3675 | 0.1485 | −0.1135 | 0.0755 | −0.3535 |
| MGAT4B | −0.136 | 0.001 | −0.057 | −0.086 | −0.118 | −0.006 | −0.087 | −0.493 | −0.162 | 0.878 |
| MGC1091 [symbol] | 0.2435 | −0.5825 | 0.0145 | −0.1125 | 0.5075 | 1.2195 | 0.6315 | 0.2695 | 0.5385 | −0.0885 |
| MGC1098 [symbol] | −0.4265 | −0.3745 | −0.6485 | 0.6425 | 0.1585 | 0.5855 | 0.7855 | 0.3575 | 0.1955 | 0.0995 |
| MGC1124 [symbol] | 0.107 | 0.131 | 0.133 | 0.352 | 0.111 | −0.286 | −0.013 | 0.049 | −0.861 | 0.033 |
| MGC1127 [symbol] | −0.213 | −0.09 | −0.051 | −0.06 | −0.172 | −0.038 | 0.813 | −0.502 | −0.08 | 0.288 |
| MGC1312 [symbol] | −0.556 | −0.286 | −0.184 | −0.167 | 0.461 | 0.435 | 0.403 | −0.236 | −0.709 | 0.075 |
| MGC1318 [symbol] | −0.219 | −0.198 | 0.078 | −0.004 | −0.077 | −0.161 | −0.299 | −0.503 | 0.262 | −0.186 |
| MGC1414 [symbol] | −0.6965 | −0.4345 | 0.0005 | −0.2635 | −0.3075 | −0.3825 | −0.1975 | 0.4435 | −0.0775 | −0.3875 |
| MGG1428 [symbol] | −0.089 | 0.1 | 0.322 | 0.185 | 0.073 | −0.249 | 0.235 | −0.447 | 1.012 | 1.412 |
| MGC1481 [symbol] | −0.477 | −0.449 | −0.457 | −0.455 | 0.505 | −0.514 | −0.442 | 0.839 | 0.529 | −0.262 |
| MGC1580 [symbol] | −0.0145 | 0.1285 | −0.3685 | 0.0285 | 0.0475 | −0.1376 | −0.1225 | 0.0465 | −0.1925 | −0.0579 |
| MGC1663 [symbol] | −0.721 | −0.407 | −0.437 | −0.248 | −0.406 | −0.028 | 0.419 | 0.126 | −0.421 | −0.464 |
| MGC1873 [symbol] | 0.878 | 0.009 | −0.244 | 0.013 | −0.125 | −0.152 | −0.459 | 0.371 | −0.099 | −0.377 |
| MGC2053 [symbol] | 0.274 | −0.084 | 0.675 | 0.183 | −0.109 | −0.289 | −0.38 | 0.296 | 0.978 | 1.175 |
| MGC2398 [symbol] | −0.156 | −0.029 | 0.703 | −0.397 | −0.301 | −0.597 | −0.712 | −0.005 | −0.803 | −0.348 |
| MGC2610 | 0.303 | −0.833 | 0.267 | −0.528 | 0.003 | −0.373 | 0.819 | −0.432 | −0.715 | −1.754 |
| MGC2714 | 0.274 | −0.991 | 0.553 | −0.468 | −0.578 | −0.868 | −0.366 | 0.464 | −0.622 | −0.927 |
| MGC2716 [symbol] | 0.5255 | −0.9925 | 0.6825 | −0.3755 | −0.3845 | −0.9245 | −0.4175 | 0.0355 | −0.8445 | −1.2375 |
| MGC2716 [symbol] | 0.2715 | −1.2525 | −0.1865 | −0.3875 | −0.2785 | −1.1645 | −0.2835 | −0.6235 | −0.9955 | −1.3155 |
| MGC2716 [symbol] | 0.331 | −1.238 | 1.212 | −0.491 | −0.677 | −1.222 | −0.401 | −0.035 | −1.382 | −1.035 |
| MGC2716 [symbol] | −0.1785 | −0.4875 | −0.5795 | −0.5235 | −0.4685 | −0.4416 | −0.2115 | −0.9465 | 0.6895 | 0.2695 |
| MGC2716 [symbol] | 0.068 | 1.393 | 0.063 | 0.274 | 0.142 | 0.824 | 0.726 | 1.06 | 0.914 | 1.010 |
| MGC2887 [symbol] | −0.095 | −0.258 | 0.498 | −0.369 | −0.498 | 0.712 | −0.04 | 1.196 | −0.464 | 0.387 |
| MGC3212 [symbol] | −0.055 | 0.943 | 0.483 | 0.57 | 0.929 | 0.776 | 0.827 | −1.23 | −0.349 | −1.027 |
| MGC3321 [symbol] | −0.3225 | −0.3235 | 0.0475 | −0.8045 | −0.5405 | −0.2675 | 0.2895 | −0.4115 | −1.1645 | −0.8295 |
| MGC3484 [symbol] | 1.2895 | −0.2785 | −0.0955 | 0.4295 | 0.7165 | 0.4735 | 0.7855 | 0.2985 | −0.2325 | −0.0995 |
| MGC3492 [symbol] | 1.478 | 0.502 | −0.373 | 0.302 | 0.278 | 2.255 | 2.216 | 2.306 | 1.072 | 1.568 |
| MGC4021 [symbol] | −0.272 | −0.569 | 0.629 | −0.749 | −0.373 | −0.32 | −0.355 | −0.186 | −0.035 | −0.789 |
| MGC4251 | 0.0495 | −0.1475 | −0.0175 | −0.6085 | −0.6165 | 0.1415 | 0.7435 | −0.0085 | −0.0995 | 1.1355 |
| MGC4308 | 1.247 | 0.639 | 0.072 | −0.093 | −0.231 | 0.895 | 0.796 | 1.496 | 0.81 | 1.193 |
| MGC4800 | 1.3445 | 0.0345 | 0.5585 | 3.2245 | 2.8885 | 1.7695 | 1.3445 | −1.2855 | −0.6605 | −1.3155 |
| MGC4659 | 0.332 | −0.116 | −0.235 | 0.829 | 0.778 | 0.508 | −0.003 | −0.886 | −1.105 | −0.772 |
| MIA | 0.0365 | −0.0165 | −0.3285 | 0.7105 | 0.7805 | 0.5425 | 0.0225 | −0.3855 | −1.0255 | −0.3525 |
| MID1 | 0.2875 | −0.1615 | 0.0195 | 0.7095 | 0.6125 | 0.3955 | −0.1855 | −1.0375 | −1.9665 | −1.0495 |
| MID1 | −1.143 | −1.168 | −0.582 | −1.628 | −1.098 | −0.978 | 0.246 | −0.56 | 0.194 | 0.243 |
| MID1 | 0.0665 | −0.2585 | 0.2575 | −0.1915 | −0.6025 | 0.1105 | 0.0205 | 0.0515 | −0.2615 | 0.8955 |
| MLF1IP | −0.5325 | −0.3505 | −1.2795 | −0.9665 | −1.1135 | −0.3525 | 0.0065 | 0.1925 | −0.5425 | −0.8605 |
| MLSTD1 | 0.132 | −0.026 | −2.273 | −0.557 | 0.681 | 1.021 | 0.919 | −0.426 | 0.754 | −1.731 |
| MMP1 | −0.072 | −0.195 | −0.524 | −0.202 | −0.257 | −0.231 | −0.456 | 0.386 | −0.378 | 0.308 |
| MMP7 | −0.3365 | −0.2735 | −0.1525 | −0.7705 | −0.9126 | −0.1205 | −0.0425 | 0.1465 | 0.5965 | 0.0785 |
| MNAT1 | 0.1995 | −0.5025 | −0.3005 | −0.4935 | −0.4705 | −1.1915 | −0.5315 | −0.1565 | −0.9815 | −0.3835 |
| MRPL14 | 0.167 | −0.107 | 0.235 | −0.311 | 1.902 | −0.065 | 0.93 | 0.092 | −0.028 | 0.461 |
| MRPL15 | 0.2305 | −0.3825 | −0.4155 | 0.0235 | −0.1165 | 0.3955 | −0.2955 | −0.1505 | 0.0015 | 0.3355 |
| MRPL27 | 0.0705 | 0.3375 | −0.0465 | −0.1475 | 0.2195 | −0.0695 | 0.0095 | −0.0605 | 0.5105 | 0.5165 |
| MRPL34 | 0.033 | 0.028 | −0.502 | 0.042 | −0.155 | −0.063 | −0.001 | −0.28 | 0.748 | 0.271 |
| MRPL43 | −0.3785 | −0.1405 | −0.1505 | −0.1105 | −0.1855 | −0.0715 | −0.4745 | −0.6205 | −0.2685 | −0.1835 |
| MRPL45 | −0.286 | −0.476 | −0.339 | −0.421 | −0.279 | −0.478 | 0.25 | −0.38 | 0.596 | 0.085 |
| MRPL48 | −0.0025 | −0.3675 | −0.4405 | −0.4175 | −0.8225 | −0.5125 | −0.0485 | −0.4055 | 0.8105 | 0.1885 |
| MRPL9 | −0.2275 | −0.1115 | −0.0325 | −0.2385 | 0.0815 | −0.0955 | 0.2645 | 0.1695 | −0.2175 | −0.2075 |
| MRPS14 | −0.848 | −0.66 | −0.079 | −0.635 | −0.364 | −0.476 | −0.629 | −0.364 | −0.148 | −0.925 |
| MRPS15 | −0.324 | −0.143 | −0.305 | −0.18 | −0.218 | −0.286 | −0.345 | −0.112 | −0.054 | −0.56 |
| MRPS17 | −0.62 | −0.291 | −0.149 | −0.183 | 0.633 | 0.271 | −0.358 | −0.132 | −0.502 | −0.387 |
| MRPS24 | 0.0015 | 0.0595 | 0.2335 | 0.1495 | 0.1175 | 0.1965 | 0.1005 | 0.1585 | −0.0855 | −0.7445 |
| MRPS25 | 0.006 | −0.217 | 0.296 | 0.219 | 0.016 | −0.589 | −0.057 | −0.162 | −0.576 | −1.215 |
| MRPS31 | −0.428 | −0.448 | −0.002 | 0.145 | 0.103 | −0.192 | −0.661 | −0.117 | −0.048 | −0.384 |
| MRPS6 | −0.4375 | −0.2155 | 0.1075 | −0.3575 | −0.4145 | −0.8095 | −0.1205 | −0.3755 | −0.0035 | −0.0165 |
| MS4A1 | 0.0005 | −0.0015 | −0.0085 | −0.1025 | −0.0935 | 0.5785 | 0.1505 | 0.2795 | 0.2455 | 0.2965 |
| MSH2 | −0.412 | −0.177 | 0.881 | 0.565 | 1.137 | 0.485 | 0.906 | 0.388 | −0.356 | −0.096 |
| MSH3 | 0.159 | 0.23 | 0.318 | −0.113 | 0.281 | 0.438 | 1.054 | −0.34 | 0.743 | −0.405 |
| MSMB | −0.15 | 0.233 | 0.239 | −0.136 | −0.122 | 0.348 | −0.084 | 0.301 | 0.109 | 0.649 |
| MTA1 | −0.3805 | 0.1585 | 0.5615 | 0.3505 | −0.1005 | −0.5785 | −0.6315 | 1.1355 | 1.6265 | 0.9555 |
| MTA3 | 0.129 | −0.065 | −0.138 | 0.006 | −0.006 | 0.135 | −0.024 | −0.244 | −0.644 | −0.143 |
| MTAC2D1 | −0.303 | −0.349 | −0.413 | −0.139 | −0.166 | −0.217 | −0.262 | 0.469 | 0.308 | −0.314 |
| MTCH1 | 0.4415 | 0.3295 | −0.1775 | 0.0745 | −0.4115 | −0.2405 | −0.6415 | −0.5445 | −1.1305 | −0.1805 |
| MTHFD1 | 1.1835 | −0.1025 | −0.2275 | −0.0475 | −0.2235 | 0.0565 | 1.1465 | 0.5605 | 1.7515 | 0.8155 |
| MTMR2 | 0.1825 | −0.5795 | 0.3715 | 0.5885 | −0.1255 | 1.9705 | 0.8165 | 0.2795 | 1.9185 | 1.4315 |
| MUC1 | −0.358 | 0.404 | −0.211 | 0.015 | 0.285 | 0.385 | 1.176 | −0.462 | −0.405 | −0.308 |
| MUC20 | 0.156 | 0.24 | −0.11 | 0.95 | 0.255 | 0.376 | 0.091 | −0.21 | −0.245 | −0.08 |
| MUM1 | 0.306 | 1.417 | 1.535 | 0.776 | 0.071 | −0.159 | 0.847 | 1.288 | −0.063 | −0.7 |
| MXI1 | −0.022 | −1.276 | −1.282 | −0.162 | −0.149 | −0.107 | 0.012 | 0.628 | −0.776 | −0.332 |
| MYB | −1.527 | −1.097 | −1.018 | −0.877 | −0.969 | −0.392 | 0.028 | −0.63 | −1.139 | −1.218 |
| MYBL1 | −1.023 | −0.683 | 0.081 | −0.188 | 1.039 | −0.634 | −0.011 | −0.971 | −0.051 | −0.422 |
| MYBL2 | 0.693 | 1.218 | 0.395 | 0.435 | −0.4 | 0.696 | 0.166 | 0.248 | 1.842 | 0.913 |
| MYO10 | −0.2845 | 0.0855 | 0.0305 | 0.2235 | 0.2785 | 0.3215 | 0.3645 | 0.6625 | 0.1885 | −0.2675 |
| MYO6 | 1.1865 | 0.1615 | −1.1175 | −0.7965 | 1.2635 | −0.7275 | 0.6305 | 0.4235 | −2.1265 | −1.5835 |
| MYST3 | −0.0245 | 0.1965 | 0.2065 | 0.2445 | 0.0135 | 0.2355 | 0.0025 | 0.3445 | −0.1725 | 0.7825 |
| NALP2 | −0.9925 | −0.2935 | 0.4015 | −0.5725 | 0.0555 | 1.0055 | −0.4755 | −0.1995 | 0.1385 | −0.7665 |
| NALP6 | 0.1115 | 0.0865 | −0.6295 | −0.1505 | 0.3735 | −0.2765 | 0.3375 | 0.1175 | −0.2295 | 0.1025 |
| NANS | 0.598 | 1.588 | 1.769 | 0.08 | 0.735 | −0.384 | 0.913 | 1.179 | 3.971 | 2.851 |
| NARF | 0.231 | 0.743 | 0.024 | −0.055 | 0.092 | 1.088 | 0.056 | −0.076 | −0.193 | 0.518 |
| NAT2 | −0.2365 | 0.0735 | −0.1835 | −0.4915 | −0.0235 | 0.1055 | 0.5895 | 0.5605 | −0.5955 | 0.0955 |
| NAV2 | 0.418 | −0.234 | 0.162 | 0.162 | 0.408 | −0.401 | 0.414 | −0.258 | 0.621 | 0.71 |
| NDE1 | −0.1605 | 0.3915 | 0.1325 | 1.2825 | 1.4475 | 1.0625 | 1.2295 | −0.4995 | −0.5145 | 0.0065 |
| NDP52 | −0.25 | −0.366 | −0.116 | −0.367 | −0.386 | −0.057 | −0.115 | 0.115 | 0.979 | 0.155 |
| NDRG2 | −0.401 | −0.272 | 0.002 | −0.327 | 0.071 | 0.189 | −0.285 | 0.56 | 1.193 | 0.152 |
| N0UFA7 | −0.1055 | −0.1885 | −0.6145 | −0.3905 | −0.0105 | 0.1945 | 0.2185 | 0.2995 | 0.1365 | 0.3405 |
| NDUFB1 | −0.248 | −0.126 | −0.011 | −0.475 | −0.142 | −0.341 | −0.021 | −0.261 | 0.307 | 0.098 |
| NDUFB10 | −0.006 | −0.055 | 0.068 | −0.013 | −0.17 | −0.65 | −0.444 | −0.414 | 0.547 | 0.078 |
| NDUFB2 | −0.1785 | 0.0495 | 0.0375 | −0.1865 | −0.0175 | −0.7245 | −0.4285 | −0.0345 | 0.4085 | −0.0465 |
| NDUFB5 | 0.2285 | −0.4505 | 0.5875 | −0.2975 | −0.1375 | −0.7555 | −0.8825 | −0.1785 | −0.7085 | −0.0645 |
| NDUFB6 | −0.1685 | −0.2035 | −0.3025 | −0.4015 | −0.5985 | 0.6495 | −0.3335 | 0.4765 | 0.3365 | 0.0575 |
| NDUFC2 | −1.3415 | −2.5985 | −0.9465 | −2.1665 | −2.1065 | −1.5335 | 0.0815 | −1.0615 | 1.1625 | −0.1125 |
| NEBL | 0.157 | 0.139 | 0.482 | 0.037 | 0.074 | 0.064 | 0.073 | 0.036 | 0.233 | −0.106 |
| NEK2 | 1.246 | 3.303 | 2.549 | −0.711 | −0.422 | −1.332 | 1.89 | 2.518 | 1.871 | 1.832 |
| NEK6 | −0.3845 | 0.6545 | −0.1545 | 0.5805 | 0.5515 | 2.0115 | 0.3955 | −0.4345 | −0.4325 | 0.3785 |
| NELL2 | 0.2845 | −1.0855 | 0.3645 | −0.5555 | −1.1255 | −0.3035 | −0.5155 | −0.0055 | −0.8885 | −0.1955 |
| NEURL | −0.333 | −0.629 | 0.511 | 0.725 | 0.592 | −0.476 | 0.693 | −0.633 | −0.166 | −1.064 |
| NFE2L3 | −0.4305 | −0.2885 | −0.4255 | 1.5065 | 1.2175 | 0.5285 | 0.1335 | −2.7935 | −1.9155 | −1.8065 |
| NFIA | 0.226 | −0.083 | 0.305 | −0.048 | 0.358 | 0.907 | 0.666 | 0.564 | 0.851 | 0.125 |
| NFIB | −0.1495 | 0.5855 | 0.4645 | 0.2385 | −0.1445 | −0.2435 | −0.1425 | −0.2365 | −0.2975 | −0.0525 |
| NINJ1 | −0.1825 | −0.3905 | −0.1055 | 0.3505 | 0.1955 | −0.1205 | −0.0665 | −0.5445 | 0.0765 | −0.0845 |
| NIPSNAP [symbol] | −0.0225 | −0.1095 | 0.2545 | −0.6855 | 1.1305 | 0.1595 | 0.1895 | −0.2775 | 0.2735 | 0.0745 |
| NKX8-2 | 0.1595 | −0.0275 | 0.0825 | −0.8415 | 1.1955 | −0.0485 | 0.1485 | −0.4265 | 0.4505 | 0.0455 |
| NME1 | 0.192 | −0.134 | 0.314 | −0.28 | −0.022 | 0.703 | 1.172 | 0.214 | 0.427 | 0.755 |
| NME2 | −1.2395 | 1.9905 | 0.4725 | −1.5845 | −1.5625 | −0.7645 | 1.3845 | 0.4975 | −3.4895 | −1.5825 |
| NME3 | −0.2095 | −0.8595 | 0.2135 | −0.1385 | −0.6225 | −1.0115 | −0.7265 | −0.5695 | −1.5955 | −1.3695 |
| NMES1 | −0.479 | −0.044 | −0.481 | −0.242 | −0.096 | −0.389 | −0.444 | 0.921 | −0.022 | −0.644 |
| NMI | 0.1155 | −0.0035 | 0.0205 | −0.1855 | −0.0055 | −0.2585 | 0.1175 | −0.0615 | −0.1805 | −0.2685 |
| NOC4 | −0.0505 | −0.3465 | −0.0835 | −0.3945 | −0.2685 | −0.5285 | −0.3695 | −0.2515 | −0.5785 | −0.5145 |
| N0LC1 | 0.1515 | 0.1195 | 0.3595 | −0.0025 | −0.1615 | −0.4035 | −0.4245 | −0.7185 | −0.5555 | 0.0465 |
| NotFound | −0.0735 | −0.0505 | −0.1705 | −0.0025 | 0.2095 | −0.2745 | 0.2455 | 0.2865 | −0.1545 | −0.0285 |
| NotFound | 0.041 | −0.839 | −0.07 | −0.121 | −0.283 | −0.726 | −0.333 | −0.195 | −0.936 | −0.458 |
| NotFound | −0.757 | −0.135 | −0.009 | 0.419 | 0.916 | 0.251 | −0.201 | −0.375 | −0.169 | 0.08 |
| NotFound | −0.255 | 0.003 | 0.217 | −0.225 | −0.177 | −0.802 | −0.387 | −0.766 | −0.505 | −0.857 |
| NotFound | 0.0735 | −0.5775 | 0.5915 | −0.6465 | −1.3075 | −0.2465 | −1.6595 | −0.5275 | −1.5625 | −1.5545 |
| NotFound | 1.233 | 0.444 | −0.557 | 1.706 | 1.967 | 1.424 | 1.656 | −0.503 | 0.239 | −0.766 |
| NotFound | 1.1445 | −2.1065 | −0.5765 | 0.0105 | −0.2365 | 0.1435 | −2.2245 | −0.9225 | 1.6705 | 0.1725 |
| NotFound | −0.757 | −0.529 | 0.015 | −0.056 | −0.207 | 0.08 | −0.069 | −0.029 | −0.901 | −1.17 |
| NotFound | −0.4255 | 0.0185 | 0.1405 | 0.2625 | 0.4925 | 0.2465 | 0.6985 | −0.5185 | −0.6435 | −0.4035 |
| NotFound | 0.5065 | −2.0955 | 0.4435 | −0.1435 | −0.2405 | −1.9355 | −0.1115 | −0.1915 | −2.4935 | −2.6155 |
| NotFound | −0.7325 | −0.3795 | −0.1205 | −1.0335 | −0.3295 | 0.8535 | 0.2175 | 0.0215 | 0.0095 | −0.1175 |
| NotFound | −0.0095 | 0.0015 | −0.1625 | 0.1095 | 0.0405 | 0.0995 | −0.1325 | 0.1385 | −0.0315 | 0.0255 |
| NotFound | −1.194 | −0.197 | 1.035 | 0.475 | 0.948 | 0.508 | 1.926 | −1.882 | −0.348 | 0.997 |
| NotFound | −0.064 | −0.035 | −0.077 | −0.162 | 0.099 | −0.149 | 0.403 | 0.152 | 0.115 | −0.201 |
| NotFound | −0.22 | −0.053 | −0.23 | −0.49 | 0.45 | 1.484 | 0.408 | 0.776 | −0.441 | −0.4 |
| NotFound | −0.2835 | −0.0085 | −0.0385 | −0.0865 | 0.1675 | 0.6045 | 0.7825 | 0.1645 | −0.4865 | 0.1965 |
| NotFound | −0.389 | 0.97 | −0.221 | 0.165 | 0.469 | 0.72 | 1.328 | 0.797 | 0.398 | 0.778 |
| NotFound | −0.396 | 0.205 | 0.249 | −0.091 | 0.427 | −0.143 | 0.306 | −0.521 | 0.236 | −0.734 |
| NotFound | −0.2245 | −0.3725 | 0.0475 | −0.2975 | −0.2635 | −0.2935 | −0.1805 | 0.0535 | −0.5565 | −0.2045 |
| NotFound | 0.9285 | 0.7995 | 0.3265 | 1.3845 | 1.4275 | 0.8545 | −0.3365 | −0.3195 | 1.8765 | 1.9495 |
| NotFound | −0.131 | 0.582 | 0.176 | 0.292 | 0.507 | 0.409 | 0.825 | 0.128 | 0.199 | −0.205 |
| NotFound | −0.0005 | 0.0345 | −0.5025 | 0.2965 | 0.5035 | 0.1045 | −0.6105 | −0.8475 | −0.8885 | −1.0455 |
| NotFound | 1.474 | 2.406 | 2.833 | 0.095 | 1.852 | −0.918 | 2.157 | 2.131 | 3.979 | 3.773 |
| NotFound | 0.322 | −1.155 | −0.698 | −0.747 | −0.559 | −0.221 | −0.932 | −0.271 | 1.069 | 0.467 |
| NotFound | 2.5805 | −0.5785 | −0.8965 | −0.4745 | −0.6315 | −0.2865 | −0.6135 | 0.1065 | −0.0215 | −0.3545 |
| NotFound | 0 | −0.176 | −0.231 | −0.278 | −0.035 | 0.049 | 0.417 | −0.455 | 0.35 | −0.14 |
| NotFound | −0.0145 | −1.4265 | −0.9905 | −0.7585 | −0.3225 | 0.1125 | −0.8695 | −0.4095 | 0.5775 | 0.0955 |
| NotFound | −0.737 | −0.365 | −0.296 | 0.132 | 0.125 | −0.822 | −0.622 | 0.665 | −0.138 | −0.554 |
| NotFound | −0.124 | 0.648 | 0.499 | 0.531 | 0.163 | −0.241 | −0.06 | 0.009 | 0.455 | 0.135 |
| NotFound | −0.2935 | −0.5065 | −0.0125 | −0.0365 | −0.4345 | −0.5405 | −0.2955 | −0.4955 | −0.9085 | −0.6565 |
| NotFound | 0.3115 | −0.4345 | −1.2635 | −0.4245 | −0.4585 | −0.6855 | 0.0125 | −0.5315 | 0.3985 | 1.8145 |
| NotFound | 0.5675 | −1.2645 | −1.0875 | 0.0235 | −0.3225 | 1.6015 | −0.5015 | 0.3515 | 0.9485 | 0.0585 |
| NotFound | −0.1065 | −1.2955 | −0.6875 | −0.7485 | −0.6065 | 0.0175 | −0.9425 | 0.0065 | 0.5375 | −0.0125 |
| NotFound | 0.1515 | 0.2405 | 0.7655 | 0.0565 | −0.1235 | −0.4995 | 0.1885 | −1.4685 | 1.0965 | 1.1475 |
| NotFound | −0.089 | −0.7 | −0.628 | 0.323 | −0.19 | −0.223 | −0.653 | −0.494 | 0.352 | 0.263 |
| NotFound | 0.6245 | −0.4245 | −0.7065 | 0.1505 | −0.4045 | 0.7675 | −0.4035 | −0.2395 | 0.7105 | 0.8325 |
| NotFound | −0.5285 | −0.2875 | −0.2965 | −0.7215 | −0.0495 | −0.5215 | −0.3365 | 0.5705 | 0.0505 | −0.4365 |
| NotFound | −0.1825 | −2.2065 | −1.4625 | −0.8505 | −1.0955 | 0.2615 | −0.1645 | −0.0325 | 1.4965 | 1.1905 |
| NotFound | 0.1465 | 0.4785 | −0.7075 | −0.1585 | 0.6555 | −0.4115 | −0.3275 | 0.3235 | 0.1815 | 1.0845 |
| NotFound | −0.315 | −0.504 | 0.116 | 0.353 | 0.074 | −0.707 | 1.347 | −0.588 | −1.432 | −0.238 |
| NotFound | 0.101 | 0.344 | −0.069 | −0.068 | 0.285 | −0.031 | 0.092 | 0.009 | 0.275 | 0.073 |
| NotFound | −0.271 | −0.634 | 0.564 | −0.408 | 0.007 | −0.499 | −0.27 | 0.012 | −0.407 | −0.633 |
| NotFound | −0.1515 | −1.4715 | −0.9045 | −0.7195 | −0.4535 | −0.0155 | −0.8965 | −0.2495 | 0.8185 | −0.0305 |
| NotFound | −0.2715 | 0.0235 | −0.4265 | −0.1225 | 0.0485 | −0.8845 | −0.4965 | 0.4745 | 0.1845 | 0.6645 |
| NotFound | −0.103 | −0.186 | −0.233 | −0.102 | −0.041 | 0.003 | 0.099 | 0.164 | 0.168 | 0.101 |
| NotFound | −0.1405 | 0.0785 | 0.1715 | −0.1455 | −0.0265 | 0.1985 | 0.0815 | 1.0285 | −0.2555 | 0.0045 |
| NotFound | −0.4315 | −0.3125 | −0.0565 | 0.3875 | 0.5225 | 0.1215 | 1.4065 | −0.5065 | 0.2835 | 1.2425 |
| NotFound | 0.2315 | 0.0085 | −0.0495 | 0.0845 | 0.2055 | 0.5655 | 0.7955 | 0.2165 | −0.1975 | 0.8215 |
| NotFound | −0.5535 | −0.4175 | 0.4235 | −0.2875 | −0.1415 | −0.1725 | −0.0505 | −0.0835 | 0.1785 | −0.1965 |
| NotFound | 0.061 | 0.697 | 0.859 | 0.81 | 0.708 | 0.271 | 0.541 | −0.109 | 0.135 | 0.412 |
| NotFound | −0.389 | 0.648 | 0.008 | −0.132 | 0.227 | 1.406 | 0.443 | 0.264 | 0.012 | 0.703 |
| NotFound | 0.0015 | −0.0015 | −0.0065 | 1.0675 | 1.0135 | 0.3965 | 0.1255 | −0.3665 | −0.0135 | 0.3375 |
| NotFound | 0.2445 | −0.0335 | 0.0895 | −0.1415 | 0.2755 | −0.0825 | −0.0235 | −0.1375 | 0.1005 | 0.3865 |
| NotFound | −0.0105 | 0.6735 | 0.0685 | 0.3805 | 0.2995 | 1.1855 | 0.8275 | 1.2265 | 0.0105 | 0.4095 |
| NotFound | 0.3815 | −0.3145 | 0.3955 | 0.0585 | −0.1425 | −0.8975 | −0.4305 | 0.6385 | −0.0095 | 0.0085 |
| NotFound | −0.995 | −0.671 | −0.881 | 0.259 | 0.202 | −0.543 | −0.65 | 1.74 | −1.618 | −2.631 |
| NotFound | 0.669 | 5.586 | 0.772 | 0.614 | 2.293 | 1.282 | 1.529 | 3.788 | −0.325 | 3.544 |
| NotFound | −0.0835 | −0.3065 | −0.2145 | 0.1015 | −0.0855 | −0.2785 | −0.2055 | −0.1345 | 0.0495 | 0.0725 |
| NotFound | −0.952 | −0.062 | 0.049 | 0.067 | 0.254 | 2.688 | −0.112 | −0.612 | −2.079 | −0.94 |
| NotFound | 0.654 | −0.137 | −0.412 | −0.179 | 0.255 | −0.523 | 0.365 | −0.488 | −0.414 | 0.171 |
| NotFound | −0.3155 | 0.8075 | 0.4985 | −0.0115 | 2.3165 | 0.0785 | 1.5895 | 1.2085 | 1.3035 | 0.0055 |
| NotFound | −0.232 | −0.058 | −0.087 | 0.077 | 0.063 | −0.329 | 0.07 | 0.106 | −0.064 | −0.423 |
| NotFound | 0.69 | −1.607 | −1.498 | −0.044 | −0.503 | 1.964 | −0.676 | 0.346 | 1.168 | 0.189 |
| NotFound | −0.7745 | 3.0605 | −0.0505 | −0.1955 | −1.0375 | 0.2915 | −1.2055 | 2.7175 | 3.0585 | 2.3425 |
| NotFound | 0.305 | 0.468 | 0.704 | −0.473 | −0.041 | 0.395 | 1.335 | 0.858 | 1.072 | 0.701 |
| NotFound | −0.1795 | −0.1995 | 0.1545 | −0.3385 | 0.2525 | −0.0885 | −0.2915 | 0.0805 | −0.5345 | −0.4745 |
| NotFound | 0.0195 | 0.6575 | −0.1195 | −0.2105 | −0.0095 | −0.0785 | 0.0305 | 0.1235 | −0.0305 | −0.0055 |
| NotFound | 0.088 | −1.381 | −1.072 | −0.683 | −0.407 | −0.117 | −0.844 | −0.012 | 0.627 | 0.221 |
| NotFound | −0.4045 | −0.7055 | −0.5385 | −0.7485 | −0.7605 | −0.7265 | −0.1235 | −0.6245 | 0.1055 | 0.1605 |
| NotFound | −0.869 | 0.622 | 0.332 | −0.005 | 0.385 | 1.14 | 0.714 | 0.363 | 0.622 | 0.592 |
| NotFound | 1.085 | 0.366 | 0.225 | 0.103 | −0.193 | −0.021 | 0.522 | 0.193 | 0.515 | 1.417 |
| NPDC1 | −1.0085 | −0.7055 | 0.1215 | −0.2305 | −1.0065 | −1.1045 | −0.0395 | 0.1995 | −0.2755 | 0.0055 |
| NPEPPS | 1.183 | −0.231 | 0.123 | −0.33 | −0.104 | −0.183 | 0.84 | −0.111 | 1.095 | 1.282 |
| NPY | 0.064 | −0.41 | −0.118 | −0.236 | −0.641 | −0.482 | −0.755 | −0.706 | −0.965 | −0.216 |
| NQO3A2 | 0.281 | 0.584 | 0.105 | 0.234 | 0.31 | 0.359 | 0.296 | −0.549 | 3.214 | 1.112 |
| NRAS | 2.762 | 1.437 | 0.709 | 1.734 | 0.678 | 0.993 | 0.355 | 0.699 | −1.898 | −0.994 |
| NRIP1 | 0.4425 | 0.7345 | 0.3775 | 0.0535 | −0.6535 | 0.3245 | −0.1855 | 0.4715 | 3.3275 | 1.5945 |
| NTN4 | −0.1035 | −0.4575 | −0.3325 | −0.1775 | −0.5445 | −0.4875 | −0.6965 | −0.1845 | −0.0295 | 0.0005 |
| NUCB2 | −0.0235 | 0.1825 | −0.5475 | −0.0985 | −0.5745 | −0.1865 | 0.1755 | 0.4015 | 0.2815 | 0.1935 |
| NUDT5 | −0.415 | −0.297 | −0.497 | −0.354 | −0.238 | −0.63 | −0.622 | −0.374 | 0.232 | −0.229 |
| NUDT6 | −0.145 | 0.174 | −0.148 | 0.259 | 0.37 | 0.023 | 0.501 | −0.034 | −0.317 | −0.183 |
| OCIAD2 | 0.1985 | 0.0075 | −0.0485 | 0.2585 | 0.4035 | 0.2355 | −0.3775 | −0.1555 | −0.8295 | −1.0995 |
| OFD1 | 0.6345 | −0.3835 | −0.2875 | −0.2405 | 0.0465 | 0.5055 | −0.1036 | −0.1225 | 0.6145 | −0.5305 |
| OGFRL1 | 0.311 | −0.148 | −0.095 | −0.099 | 0.115 | 0.444 | 0.177 | 0.348 | 0.325 | 0.205 |
| OPLAH | 0.0845 | −0.3205 | 0.0285 | −1.0525 | 0.0755 | 1.0325 | −0.2045 | −0.1805 | −0.8735 | −0.7795 |
| ORMDL3 | 1.013 | 0.179 | 0.441 | −0.007 | 0.332 | 0.065 | −0.727 | −0.834 | −1.952 | −0.842 |
| OSBPL3 | 0.125 | −0.205 | −0.365 | −0.019 | −0.28 | −0.677 | −0.511 | −0.188 | −0.343 | −0.448 |
| OSR2 | 0.0035 | −0.2385 | 0.1695 | −0.1025 | −0.2625 | −0.1365 | −0.3335 | −0.1645 | 0.2395 | −0.2695 |
| OSTF1 | 0.43 | −0.352 | 0.224 | −0.445 | 0.152 | 2.077 | −0.547 | −0.81 | −0.798 | −1.565 |
| P2RX1 | −0.3195 | −0.6075 | 0.2675 | −0.2575 | 0.1255 | −0.1945 | −0.1015 | −0.5925 | −1.1185 | −0.9745 |
| P2RY2 | −0.487 | −0.62 | −0.446 | 0.352 | 1.551 | 0.487 | 0.199 | −0.198 | −0.494 | −0.798 |
| PACAP | −0.35 | 0.199 | −0.16 | −0.499 | 0.127 | 0.292 | −0.142 | 1.017 | 1.306 | −0.896 |
| PADI2 | −0.2195 | 0.0415 | −0.2575 | 1.1405 | 1.1835 | 0.1335 | −0.6425 | 2.8255 | −0.3785 | −0.6075 |
| PAPD5 | −0.069 | 0.288 | −0.026 | 0.007 | 0.033 | 0.725 | −0.278 | 1.093 | 1.496 | 0.336 |
| PAPSS2 | −0.441 | −0.685 | −0.459 | −0.826 | −0.787 | −0.93 | −0.001 | −0.486 | 0.257 | 0.019 |
| PARD8A | −0.066 | 0.203 | 0.213 | −0.992 | −0.616 | −0.732 | −0.343 | 0.597 | −0.51 | −0.158 |
| PARP1 | −0.394 | 0.848 | −0.093 | −0.213 | 0.01 | 0.394 | 0.637 | 0.056 | 0.033 | −0.179 |
| PARP9 | 0.5585 | 0.8205 | 0.2055 | 0.5095 | 0.7055 | 0.2415 | 0.2075 | −0.1545 | 3.8455 | 0.5295 |
| PAXIP1L | −0.1615 | 0.2595 | −0.0355 | 0.0715 | 0.8095 | −0.0116 | −0.4805 | −0.1425 | −0.9065 | −0.2855 |
| PBX3 | 0.1905 | 0.2305 | 0.5645 | 0.2085 | 0.4205 | 0.3755 | 0.0625 | −0.1905 | −0.7715 | −0.4755 |
| PCDHB11 | −0.425 | 0.024 | −0.612 | −0.848 | 1.062 | 0.058 | −0.977 | −1.203 | −2.29 | 0.739 |
| PCDHB16 | −0.218 | 2.228 | 0.575 | 0.37 | 0.357 | 0.234 | 1.033 | −0.106 | 1.178 | 1.344 |
| PCDHB2 | 0.1525 | −0.5425 | −0.3555 | −0.4365 | −0.2535 | −0.5425 | 0.1026 | −0.1255 | 0.2615 | −0.0155 |
| PCM1 | 0.2595 | −0.0755 | 0.1465 | −0.1805 | −0.0805 | −0.1025 | −0.1855 | −0.0175 | −0.3655 | −0.2435 |
| PCNA | −0.007 | −0.385 | −0.278 | −0.307 | −0.739 | −0.77 | −0.431 | −0.544 | −0.025 | −0.528 |
| PDC01 | 0.5075 | 0.4375 | 0.7515 | 0.3805 | 0.8255 | 1.0965 | 0.0025 | −0.1335 | −1.5605 | −1.8295 |
| PDC010 | −0.448 | −0.429 | −0.203 | −0.414 | −0.407 | −0.686 | −0.787 | −0.482 | 0.004 | −0.506 |
| PDCD4 | −0.2745 | −0.4785 | 0.0395 | −0.2155 | −0.0035 | −0.1155 | −0.2685 | 0.1885 | −0.2305 | −0.3705 |
| PDCD5 | 0.9175 | 1.6095 | 0.0365 | 0.4195 | −0.5845 | −0.4285 | 0.4425 | −0.3195 | 1.5355 | 1.0685 |
| PDCD8 | −0.4705 | −0.2675 | 0.0845 | −0.2215 | −0.4125 | −0.1425 | −0.3405 | −0.1185 | −0.2625 | −0.1465 |
| PDE4DIP | 0.4075 | 0.1595 | 0.7125 | 0.8685 | 0.3735 | 0.2525 | 0.2055 | 0.4125 | 0.1655 | 0.0555 |
| PDHA1 | −1.369 | −2.104 | 1.405 | 1.785 | 0.189 | 0.646 | −1.861 | 0.432 | −2.454 | −1.32 |
| PECI | 0.4855 | 0.3145 | 0.2025 | 0.6815 | 0.3465 | 0.4755 | 0.0125 | −0.0975 | 0.6775 | −0.4216 |
| PEG10 | −0.2265 | −0.4605 | −0.0245 | 0.6805 | 0.7955 | −0.4755 | −0.2875 | −0.4455 | 0.5095 | −1.8135 |
| PER2 | −0.342 | −0.036 | −0.057 | −0.083 | 0.245 | −0.033 | 0.065 | −0.364 | 0.282 | −0.045 |
| PERP | 0.3585 | 0.2805 | 0.3435 | 0.4245 | −0.2125 | 0.6705 | 0.3525 | 1.2015 | −0.1535 | −0.4105 |
| PEX1 | −0.283 | −0.685 | −0.298 | −0.595 | −0.479 | −0.638 | −0.263 | −0.504 | 0.82 | 0.23 |
| PFAAP5 | 0.1095 | 0.8245 | 0.0625 | 1.4485 | 0.7145 | −0.4805 | −0.3765 | −0.6175 | −0.8535 | 0.4795 |
| PFDN2 | 0.022 | −0.071 | −0.189 | −0.25 | 0.819 | 0.27 | 0.514 | 0.073 | −1.062 | 0.369 |
| PGM1 | −0.3435 | −0.0795 | −0.1285 | −0.3785 | −0.2445 | −0.1445 | −0.0855 | 0.0685 | −0.0505 | −0.3555 |
| PGR1 | −0.7055 | −0.6535 | −0.0795 | 0.0855 | 0.4545 | −0.4665 | −0.3185 | −0.6055 | −0.4735 | −0.4035 |
| PHAX | 0.269 | −0.24 | −0.68 | −0.424 | −0.625 | −0.205 | −0.015 | 0.32 | 0.339 | 0.437 |
| PHGDH | 1.2425 | 0.6225 | 0.7765 | 0.5915 | 0.8285 | 0.1125 | 1.2215 | 1.5065 | −1.4935 | 0.2745 |
| PHYH | 0.3215 | 0.1655 | −0.1055 | −0.5615 | −0.1465 | 0.5205 | 0.2115 | −0.3105 | 0.4755 | 0.2365 |
| PI85PA | 0.2285 | −0.9225 | 0.1365 | −0.5895 | −0.3385 | −0.9475 | −0.0705 | −0.0765 | −0.6195 | −0.4725 |
| PIGT | −0.041 | 0.005 | 0.121 | 0.06 | 0.32 | 0.513 | 0.324 | 0.747 | −0.077 | 0.431 |
| PIM2 | −0.8545 | 0.3175 | −0.0495 | −0.4115 | 0.2545 | −0.3015 | 0.8855 | 0.4945 | −0.9505 | −0.4135 |
| PISD | 1.725 | 1.183 | 0.977 | 0.867 | −0.086 | −0.879 | −0.423 | 1.189 | 2.345 | 3.915 |
| PITPNC1 | −1.2315 | −1.2785 | −0.9425 | −1.3405 | −0.9885 | −0.7565 | 0.1965 | −0.0845 | −0.3995 | −0.5725 |
| PKIB | 0.581 | 0.37 | −0.477 | 0.253 | 0.329 | 0.798 | 0.462 | 0.185 | 0.199 | −0.009 |
| PKMYT1 | 0.972 | −0.583 | 0.445 | 0.274 | 0.134 | −0.237 | −0.502 | −0.739 | −1.865 | −0.802 |
| PLA2G4B | −0.2705 | −0.3065 | −0.1885 | 0.9435 | 1.7225 | 1.0035 | 0.8035 | −1.3875 | −0.9035 | −0.5205 |
| PLAC8 | 0 | −0.887 | −0.213 | 0.35 | −0.01 | 0.227 | −0.032 | 0.62 | −0.272 | 0.14 |
| PLCB1 | −0.3285 | −0.2645 | −0.1445 | −0.4775 | −0.4255 | −0.8835 | −0.2285 | −0.4255 | −0.1535 | 0.3935 |
| PLCXD1 | −0.101 | 0.273 | 0.023 | −0.564 | −0.028 | −0.087 | −0.002 | −0.338 | −0.43 | −0.259 |
| PLEK2 | −0.3135 | −0.2235 | −0.4085 | −0.6375 | −0.5965 | −0.7065 | −0.0495 | −0.5055 | 0.1755 | 0.1355 |
| PLO0 | 1.0915 | −0.2905 | −0.1645 | 0.0375 | −0.4615 | −0.1325 | −0.0305 | 0.3825 | −0.4305 | 0.1845 |
| PNAS-4 | 0.0145 | 0.0045 | −0.2085 | −0.5075 | 0.0425 | 0.1705 | −0.0095 | −0.8155 | −0.6535 | −0.5035 |
| PNLIPRP2 | −0.5455 | 0.4645 | −0.0315 | −0.3995 | −0.1865 | −0.7425 | −0.1155 | 1.1635 | 0.4295 | 0.6575 |
| PNMA3 | −0.0475 | −0.2755 | 0.0195 | −0.0255 | −0.9185 | −0.0605 | −0.1295 | 0.1785 | 0.0785 | −0.3045 |
| POLB | −0.252 | −0.195 | −0.358 | −0.465 | −0.134 | 0.194 | 0 | −0.113 | −0.151 | −0.189 |
| POLR2F | −0.3846 | 0.2625 | −0.2775 | −0.1645 | −0.4605 | 0.1505 | −0.4375 | −0.0005 | −0.4545 | −0.6145 |
| POLR2H | −0.1475 | −0.3995 | 0.2185 | −0.4475 | −0.3995 | −0.3955 | −0.1225 | −0.0505 | 0.2335 | −0.3225 |
| POMC | −0.261 | −0.54 | −0.176 | −0.418 | −0.061 | −0.227 | −0.031 | −0.328 | 0.159 | −0.186 |
| POP4 | −0.154 | 0.246 | 0.331 | 0.29 | 0.008 | 0.135 | 0.656 | 0.48 | 0.038 | −0.221 |
| POP7 | 0.151 | 0.148 | −0.248 | −0.213 | −0.016 | 0.239 | −0.078 | −0.313 | 0.904 | 0.064 |
| PP1057 | −0.7925 | −0.4655 | −0.1715 | −0.0125 | 0.3435 | 0.0785 | −0.3075 | −1.6725 | −0.5035 | −1.3655 |
| PPFIA1 | 0.209 | −0.239 | −0.804 | −0.539 | −0.43 | 0.261 | −0.221 | −0.6 | 0.601 | 0.185 |
| PPFIBP2 | −0.1425 | −0.1905 | 0.0475 | −0.4275 | −0.1835 | 0.2395 | 0.2845 | 0.4235 | 0.1225 | −0.4525 |
| PPGB | −0.2925 | −0.2085 | −0.1055 | 0.2895 | 0.2085 | 1.2235 | −0.3155 | −0.0115 | 0.2135 | −0.5515 |
| PPHLN1 | −0.0965 | −0.1715 | −0.3475 | −0.1605 | −0.2465 | −0.1855 | −0.4855 | 0.1275 | −0.0285 | −0.5035 |
| PPIF | −0.3005 | −0.2895 | −0.0575 | −0.0165 | −0.0575 | 0.0645 | 0.9885 | −0.1995 | 0.5355 | 0.5135 |
| PPIL1 | −0.0405 | −0.5105 | −0.1115 | −0.2985 | −0.3435 | 0.1415 | 0.0795 | −0.1315 | −0.8765 | −0.2065 |
| PPOX | 0.615 | 0.16 | 2.218 | 1.547 | −0.359 | 1.715 | 2.083 | 0.703 | −0.699 | 1.3 |
| PPP1R16 [symbol] | 0 | −0.028 | −0.208 | −0.237 | 0.181 | −0.064 | 0.487 | 0.103 | 0.075 | −0.043 |
| PPP1R3C | 0.14 | 0.511 | −0.002 | −0.158 | −0.614 | −0.008 | 0.466 | −0.747 | 1.913 | 1.326 |
| PPP2R2D | 0.0905 | −0.9545 | −0.0905 | 0.3145 | −1.1805 | −1.0105 | −0.7185 | −0.3275 | −1.8705 | 0.2005 |
| PPP2R5A | −0.857 | −0.353 | −0.552 | −0.893 | −0.214 | −0.011 | 0.4 | −0.253 | 0.277 | −0.124 |
| PRAME | −0.5845 | −0.5835 | −0.1155 | −0.4505 | −0.4416 | −0.4115 | −0.0475 | −0.0525 | 0.0625 | −0.4775 |
| PRC1 | 0.283 | −0.073 | 0.171 | 0.445 | 0.272 | −0.191 | −0.43 | −0.287 | −0.231 | 0.184 |
| PRIM2A | 0.0105 | 0.0885 | 0.4805 | 0.1255 | −0.2805 | −0.8175 | −0.5315 | −0.7755 | −1.9245 | −1.0455 |
| PRKACB | −0.23 | −0.001 | −0.265 | −0.153 | 0.306 | −0.057 | −0.028 | −0.656 | 0.271 | −0.34 |
| PRKCB1 | −0.061 | 0.032 | 0.322 | 0.168 | 0.413 | −0.484 | −0.234 | −0.22 | −1.099 | −0.288 |
| PRKCI | −0.455 | −0.022 | −0.166 | −0.259 | 0.002 | 0.382 | 0.539 | 0.424 | 0.036 | 0.197 |
| PRKRIR | 0.1905 | −0.1835 | −0.1115 | 0.3435 | 0.7585 | 1.2125 | −0.0135 | −0.8975 | −1.0615 | −0.8495 |
| PRKWNK1 | 0.7165 | 0.1705 | 0.2905 | 0.8785 | 0.9215 | 0.1865 | 0.0855 | −1.8355 | −1.7005 | −1.9175 |
| PRKX | −0.058 | −0.327 | 0.2 | 0.011 | −0.198 | −0.19 | 0.001 | 0.157 | −0.017 | −0.044 |
| PRNP | 0.0465 | −0.1575 | 0.0705 | −0.3415 | −0.4515 | −0.5155 | −0.2655 | −0.1415 | 0.0015 | −0.5585 |
| PROL5 | 0.2535 | 0.1445 | −0.8385 | 0.0695 | 0.4995 | −0.2115 | 0.6225 | −0.5255 | −0.0045 | 0.2675 |
| PRPF18 | −0.0095 | −0.1835 | 0.4905 | 0.3975 | 0.4475 | 0.2055 | 0.0915 | −0.5185 | −0.8175 | −0.0435 |
| PRPSAP1 | −0.9445 | 0.0915 | −0.8505 | 0.9205 | −0.1285 | 0.2615 | −0.4645 | 1.4645 | −0.1775 | 0.4305 |
| PRSS12 | −0.4305 | 0.6545 | 1.2115 | −0.0195 | 1.0235 | 0.8455 | 2.6885 | 1.1185 | 3.7885 | 3.0755 |
| PRSS16 | −0.2805 | −0.3445 | 0.0875 | −0.3545 | −0.4545 | −0.2995 | −0.8735 | 0.4315 | 0.4865 | −0.1365 |
| PSD3 | −0.8105 | −0.6625 | −0.5845 | −1.1595 | −0.5705 | −0.2525 | −0.5315 | −0.0705 | −0.7485 | −0.6605 |
| PSMA3 | 0.02 | −0.01 | −0.104 | −0.273 | 0.132 | 0.032 | 0.05 | 0.341 | −0.272 | 0.224 |
| PSMA7 | 0.122 | −0.184 | −0.164 | −0.006 | −0.389 | −0.030 | −0.676 | 0.051 | −0.084 | 0.093 |
| PSMC5 | −0.2115 | −0.2525 | −0.0515 | −0.0035 | −0.1516 | −0.4895 | −0.6035 | −0.0945 | 0.5935 | −0.0705 |
| PSMC6 | 0.134 | −0.267 | −0.232 | −0.821 | −0.209 | −0.449 | −0.283 | −0.32 | 0.015 | 0.06 |
| PSMD12 | −0.0995 | −0.2695 | −0.4405 | −0.3155 | 0.9135 | 0.1335 | −0.0035 | 0.1645 | 0.1585 | 0.1165 |
| PSMD12 | 0.4275 | −0.0525 | 0.6965 | −1.6335 | 0.1725 | −1.8345 | −1.8295 | 0.2445 | 0.5855 | −1.3495 |
| PSMD3 | −0.0485 | 0.0005 | 0.1585 | 0.5105 | −0.3275 | −0.4815 | −0.1975 | 0.7975 | −0.2105 | 0.0445 |
| PSPH | 0.683 | −0.001 | −0.08 | 0.167 | −0.104 | 0.155 | 0.517 | 0.443 | −0.44 | 0.001 |
| PTD012 | 3.852 | −0.194 | 1.425 | 0.617 | 3.47 | 0.084 | 1.219 | −0.148 | 2.96 | 4.051 |
| PTGER3 | 0.426 | −0.159 | −0.63 | −0.591 | −0.332 | 0.15 | 0.01 | 0.069 | 0.797 | 0.273 |
| PTHLH | 0.0275 | −0.1425 | −0.3865 | 0.4895 | 0.4915 | −0.0005 | −0.0655 | −0.9735 | −0.5595 | −0.2265 |
| PTK6 | −0.004 | −0.359 | 0.141 | −0.463 | −0.588 | −0.267 | −0.154 | −0.614 | −0.921 | −0.628 |
| PTPN4 | −0.248 | −0.487 | −0.177 | 0.029 | 0.244 | −0.258 | −0.003 | −0.509 | 0.126 | −0.474 |
| PTPN7 | 0.272 | 2.356 | 0.385 | 0.399 | −0.587 | 1.386 | −0.21 | −0.091 | 3.604 | 2.577 |
| PTPRF | 1.576 | 2.231 | 0.302 | 0.278 | 0.63 | 0.592 | 1.752 | 1.895 | 1.244 | 0.299 |
| PTPRN2 | 0.4865 | 0.2345 | 0.1215 | −0.3265 | −0.1205 | −0.3635 | −0.7925 | 0.2535 | −0.4665 | −0.2655 |
| PTPRT | −0.8385 | −0.8175 | −0.7755 | −1.2925 | −0.5665 | −0.3985 | −0.0675 | −0.3925 | 0.0115 | −0.2655 |
| PTS | −0.289 | −0.63 | −0.108 | −0.416 | −0.769 | −0.044 | −0.597 | −0.588 | 0.149 | −0.268 |
| PTTG1 | −0.1715 | −0.2075 | −0.3435 | −0.0735 | −0.1805 | −0.6105 | 0.1885 | 0.0915 | 0.3845 | −0.3995 |
| PURB | −0.1105 | 0.3245 | 1.0435 | −0.5025 | −0.3445 | −0.4875 | 0.2065 | −0.6355 | 0.2335 | 0.0435 |
| PXMP2 | −0.2875 | −0.8145 | −0.5705 | −1.1815 | 0.1465 | −0.6475 | −0.1465 | −0.2325 | 0.1345 | −0.8175 |
| PYCARD | −0.047 | 0.395 | 0.541 | −0.002 | 0.399 | −0.531 | 1.147 | 1.372 | 1.181 | 2.429 |
| PYCR1 | 0.137 | −0.49 | −0.113 | 0.048 | 0.574 | 0.833 | −0.596 | −0.386 | −0.069 | 0.992 |
| QDPR | 0.8716 | 0.4065 | −0.1555 | −0.6855 | −0.1485 | 0.5065 | 1.9785 | 0.1255 | −1.4385 | 1.0705 |
| QPRT | 0.476 | −0.938 | −0.454 | −0.435 | 0.843 | 0.269 | −0.076 | 1.124 | 0.405 | 0.377 |
| QSCN9 | −0.0005 | −0.7195 | 0.0035 | −0.6055 | −0.2445 | −0.3285 | −0.3625 | −0.6765 | 0.0475 | 0.6305 |
| RAB11FIP | 0.066 | 0.175 | −0.317 | −0.524 | −0.015 | −0.093 | 0.4 | 0.847 | 0.453 | 0.582 |
| RAB11FIP | −0.23 | 0.128 | −0.263 | −0.741 | −0.248 | −0.23 | −0.319 | 0.034 | −0.6 | −1.075 |
| RAB13 | 1.216 | −0.156 | 0.493 | 0.265 | −0.077 | −0.28 | −0.058 | −0.208 | −0.216 | 1.3 |
| RAB27A | 0.921 | 0.169 | −0.409 | 0.093 | −0.385 | 0.134 | 0.379 | 0.564 | −1.007 | 1.008 |
| RAB30 | −0.2285 | −0.1995 | −0.3355 | −1.2195 | −0.3255 | −0.1495 | −0.1615 | −0.5795 | −0.2735 | −0.5555 |
| RAB34 | −0.251 | 0.132 | 0.064 | −0.033 | 0.127 | −0.07 | −0.522 | 0.938 | −1.4 | 0.179 |
| RAB35 | −0.109 | −0.3 | 0.143 | −0.219 | −0.312 | −0.49 | 0.051 | 0.287 | 0.737 | 0.014 |
| RAB39B | −0.1485 | 0.1925 | 1.0515 | 0.2805 | −0.2785 | −0.2335 | −0.0395 | 1.4195 | 0.7825 | −0.3085 |
| RAB3A | 0.421 | 2.511 | 1.482 | −0.648 | 1.102 | 1.048 | −0.443 | 2.071 | −0.448 | 2.533 |
| RAB3IP | 0.153 | −0.404 | 0.045 | −0.211 | −0.285 | −0.142 | −0.247 | −0.447 | 0.893 | −0.421 |
| RABEP1 | −1.301 | −0.403 | −0.4 | −1.533 | −1.003 | −0.528 | −0.454 | −0.729 | 0.222 | −0.33 |
| RABGAP1 | 0.0615 | 0.4135 | −0.0705 | −0.0025 | 0.2065 | −0.0105 | 0.2805 | −0.0695 | 0.3735 | 0.2735 |
| RACGAP1 | −0.7365 | −0.9645 | −1.3415 | −0.9955 | −0.8835 | −1.0815 | 0.0505 | −1.1525 | −0.4885 | −0.3245 |
| RAD17 | −0.433 | −1.255 | −0.169 | −0.508 | −1.101 | −1.097 | −0.284 | −0.45 | −0.092 | −0.024 |
| RAD51 | 0.151 | 0.055 | −0.109 | 0.042 | −0.14 | 0.169 | −0.148 | 0.245 | 0.109 | 0.199 |
| RAD51AP | −0.9976 | −1.0645 | −0.5865 | −1.0905 | −0.9955 | −1.0545 | 0.0355 | −0.0795 | −0.7875 | −0.3135 |
| RAD52 | −1.176 | −1.632 | −1.985 | −1.426 | −1.23 | −1.299 | 1.149 | −0.046 | 1.558 | 0.074 |
| RAD54L | −0.3415 | 0.0885 | −0.3055 | −0.1715 | −0.0145 | −0.0095 | 0.4625 | 0.2405 | −0.5885 | −0.0635 |
| RAMP | −0.5255 | 0.5195 | −1.5365 | 0.2105 | 0.6665 | 2.7245 | 1.0145 | −2.2495 | −0.5885 | −1.7625 |
| RANBP1 | −0.3915 | 0.1235 | 0.2825 | −0.5145 | −0.2815 | 0.1235 | −0.0825 | 0.4665 | −0.0825 | −0.2325 |
| RARRES1 | 1.817 | 1.102 | 0.48 | 0.884 | 0.788 | −0.355 | 0.005 | 1.821 | 0.767 | 1.954 |
| RBBP7 | −0.4235 | −0.2635 | −0.0935 | −0.6195 | −0.1615 | −0.3605 | 0.7175 | 0.0545 | −0.4205 | −0.0425 |
| RBBP8 | −0.1755 | −0.0745 | 0.1055 | −0.0465 | −0.1685 | −0.0775 | 0.1965 | 0.4595 | 0.1655 | −0.3485 |
| RBM8A | 0.674 | 1.121 | 0.413 | 0.152 | 0.784 | −0.044 | −0.081 | 1.812 | 1.798 | 0.543 |
| RBX1 | −0.024 | −0.065 | 0.283 | 0.378 | 0.266 | −0.224 | 0.094 | −0.272 | −0.019 | 0.053 |
| RDHE2 | 0.172 | −0.042 | −0.098 | 0.282 | −0.164 | 0.106 | −0.143 | 0.015 | −0.057 | 0.019 |
| REA | −0.099 | −0.042 | 0.034 | −0.11 | −0.129 | −0.104 | −0.077 | 0.056 | −0.218 | −0.009 |
| REC14 | −0.3925 | −0.7145 | 0.0205 | −0.8905 | −0.4675 | −0.7005 | 0.0215 | −0.1515 | −0.8145 | −0.4175 |
| REG1A | −0.096 | −0.172 | 0.52 | 0.023 | 0.008 | −0.01 | −0.104 | −0.089 | −0.145 | 0.799 |
| RELB | −0.264 | 1.505 | 0.251 | −0.213 | −0.935 | −0.562 | 0.342 | 1.56 | 1.583 | 0.896 |
| REPIN1 | 0.305 | 1.322 | 0.425 | −0.038 | 0.187 | 0.376 | 1.176 | 1.084 | 0.975 | 0.611 |
| REPS2 | −0.7305 | −0.5945 | −0.2825 | −0.6845 | −0.4775 | −0.9505 | −0.1885 | −0.5415 | 0.1855 | −0.2995 |
| RERG | 0.1625 | 0.0495 | 0.4085 | −0.3395 | −0.7085 | −0.6545 | −0.1105 | −0.3785 | 0.2795 | −0.4815 |
| RFC4 | −0.4905 | −0.2225 | 0.2035 | −0.2905 | −0.4105 | −0.7175 | 0.0415 | −0.4995 | −0.0305 | −0.4515 |
| RGS10 | −0.5675 | −1.0675 | −0.2845 | −0.5235 | −0.4015 | −1.1385 | 0.4915 | 0.8385 | 0.3235 | 0.0935 |
| RH8DL2 | 0.028 | −0.095 | −0.323 | −0.181 | 0.081 | 0.179 | −0.316 | 0.192 | −0.141 | 0.033 |
| RIBC2 | −0.116 | −0.531 | −0.173 | −0.616 | −0.619 | −0.19 | −0.238 | −0.68 | 0.106 | 0.177 |
| RIC-8 | −0.076 | 0.636 | −0.125 | 0.028 | −0.498 | 0.142 | −0.631 | 0.837 | 1.22 | 6.204 |
| RIT1 | −0.114 | 0.13 | 0.174 | −0.254 | 0.215 | 0.229 | 0.214 | −0.235 | −0.277 | −0.258 |
| RLN2 | 0.897 | −0.036 | 0.085 | −0.148 | −0.408 | 0.18 | 0.092 | 0.377 | 0.41 | 0.455 |
| RNF10 | −0.2165 | −0.1375 | 0.2515 | −0.2695 | −0.6115 | −0.0845 | 0.0085 | 0.1285 | −0.5075 | −0.4265 |
| RNF135 | 0.0075 | −0.2035 | 0.3195 | 2.4205 | 1.8795 | 0.5185 | 1.2655 | −0.6515 | −0.8665 | −0.0605 |
| RNF7 | −0.586 | −0.426 | −0.451 | −0.284 | −0.224 | −0.326 | −0.272 | 0.161 | 0.382 | 0.017 |
| ROPN1 | 2.9975 | 0.5175 | 1.0776 | 0.4905 | 1.2415 | 0.1355 | −0.0055 | 4.5645 | −1.2375 | −1.1585 |
| RPA3 | −0.1885 | 0.2305 | 0.5865 | 0.0845 | 0.2115 | −0.0925 | 0.1845 | −0.3265 | 0.5205 | −0.4775 |
| RPESP | −0.01 | 0.684 | 0.144 | 0.507 | 1.052 | 0.122 | 0.444 | 0.92 | −0.036 | −0.695 |
| RPL11 | −0.309 | 0.28 | 0.258 | −0.006 | 0.732 | 0.074 | 0.381 | −0.163 | 0.153 | −0.573 |
| RPL13 | 0.195 | 0.515 | 0.522 | 0.71 | 0.319 | −0.014 | 0.153 | 0.071 | 0.43 | −0.035 |
| RPL13A | −0.1105 | −0.0065 | 0.5825 | 0.1775 | 0.6045 | −0.0775 | −0.1135 | −0.0315 | 0.1905 | −0.5185 |
| RPL15 | 0.14 | 0.002 | −0.031 | −0.319 | 0.822 | 0.004 | −0.121 | −0.228 | 0.525 | 0.092 |
| RPL18A | 0.034 | 0.325 | 0.791 | 0.111 | 0.628 | −0.126 | 0.382 | −0.005 | −0.09 | −0.782 |
| RPL19 | 0.066 | 0.251 | 0.074 | −0.506 | 0.192 | 0.072 | 0.084 | −0.626 | 0.48 | −0.026 |
| RPL21 | 0.1765 | 0.0315 | 0.1405 | −0.2795 | 0.0275 | −0.0015 | 0.0065 | −0.5815 | 0.3685 | 0.0795 |
| RPL23A | −0.16 | 0.448 | 0.381 | 0.221 | 0.407 | 0.083 | 0.235 | −0.267 | −0.086 | −0.807 |
| RPL23A | −0.0515 | 0.2585 | 0.2035 | 0.2525 | 0.7405 | 0.1175 | 0.3795 | 0.1195 | 0.3375 | −0.2195 |
| RPL26L1 | −0.2175 | 0.1195 | 0.7135 | 0.0595 | 0.4515 | −0.2005 | 0.4785 | −0.1195 | 0.4515 | −0.3715 |
| RPL29 | −0.2935 | 0.0385 | −0.0865 | 0.0365 | 0.0415 | −0.2405 | −0.1105 | 0.1155 | 0.2725 | −0.3065 |
| RPL35 | −0.3865 | 0.2255 | 0.1755 | −0.4505 | 0.5135 | −0.1095 | 0.1645 | −0.3075 | 0.1985 | −0.6495 |
| RPL35AL | 0.2345 | −0.9235 | 0.7135 | 0.5005 | 0.0725 | −0.0555 | 0.0705 | 5.8115 | −0.6835 | 0.5185 |
| RPS16 | 0.3085 | 0.2975 | −0.2855 | 0.0355 | 0.0635 | −0.3915 | 0.0645 | 0.1685 | −0.1705 | 0.3025 |
| RPS4Y1 | −0.4265 | −0.4035 | −0.4195 | −0.0385 | −0.9155 | −0.0185 | −0.3715 | −0.9105 | 0.9865 | 0.0295 |
| RRAGA | −0.287 | 0 | −0.616 | −0.4 | −0.254 | −0.069 | −0.117 | −0.423 | 0.618 | 0.191 |
| RRAGD | −1.396 | −0.914 | −0.826 | −2.282 | −0.394 | −1.337 | −0.24 | −1.233 | 0.073 | −0.815 |
| RRM1 | −0.137 | −0.017 | 0.402 | −0.221 | 0.064 | 0.228 | 0.543 | −0.39 | 0.233 | −0.396 |
| RRM2 | 0.777 | 0.615 | −0.048 | 1.155 | 0.38 | 1.109 | −0.005 | 0.053 | −1.849 | −1.759 |
| RSAFD1 | 0.4815 | −0.2035 | −0.1925 | −0.0105 | −0.1555 | −0.1425 | −0.2435 | −0.3085 | −0.8715 | −0.4345 |
| RSNL2 | 0.1585 | 0.0045 | −0.1645 | −0.1165 | 0.2585 | −0.6085 | −0.2355 | −0.3235 | 0.4145 | −0.0675 |
| RSU1 | −0.202 | −0.498 | −0.384 | −0.565 | −0.189 | 0.029 | −0.355 | −0.604 | −0.73 | −0.53 |
| RTN4IP1 | 0.289 | −0.5 | −0.531 | −1.817 | −0.491 | 0.927 | 0.622 | 0.741 | −1.967 | 0.089 |
| S100A11 | 0.239 | −0.215 | −0.393 | −0.548 | −0.071 | 0.659 | 0.378 | 0.355 | −1.578 | −0.415 |
| S100A14 | 0.1445 | 1.3215 | 0.3395 | 0.2125 | 0.5815 | −1.2775 | −0.4845 | −2.0525 | 1.5585 | −1.6305 |
| S100A16 | −0.2875 | −0.0475 | −0.1155 | −0.4745 | 0.0735 | −0.0905 | 0.2995 | −0.1645 | −0.4505 | −0.1655 |
| S100A9 | 0.47 | 1.069 | 0.918 | 0.441 | 0.499 | 0.285 | 0.896 | 1.522 | 0.311 | 0.76 |
| SAE1 | −0.1765 | 0.0625 | −0.3475 | −0.5055 | −0.3035 | −0.1015 | −0.1415 | 0.0745 | −0.4345 | −0.6305 |
| SALL2 | 0.143 | −0.426 | 0.219 | 0.08 | −0.238 | −0.015 | −0.723 | −0.681 | 0.171 | 0.2 |
| SARA1 | −0.0875 | 0.0405 | 0.2935 | 0.2585 | 0.2045 | 0.2555 | 0.2755 | −0.9155 | −0.4635 | 0.0055 |
| SARA2 | −0.263 | 0.039 | −0.023 | −0.445 | 0.584 | 0.489 | −0.044 | 0.254 | −0.402 | −0.545 |
| SAS | 0.798 | 0.148 | −0.035 | 0.937 | 0.017 | 1.806 | −0.407 | −1.738 | −1.258 | −0.654 |
| SCIN | 0.6355 | −0.2355 | −0.0245 | 0.6735 | 0.7295 | 0.7945 | 0.3065 | −2.0595 | −0.5565 | −1.2185 |
| SCML1 | 0.591 | −0.424 | −0.688 | −0.083 | −0.013 | 0.105 | 0.249 | −0.395 | −0.318 | −0.105 |
| SCPEP1 | 0.8205 | −1.3685 | 0.2775 | −0.8585 | 0.4245 | −0.6405 | −0.9205 | −0.0675 | −0.5185 | −0.3515 |
| SCRIB | −0.04 | 0.775 | 0.161 | −0.182 | 0.361 | −0.625 | −0.479 | 0.673 | −0.5 | −0.593 |
| SDC1 | 0.506 | −0.584 | −0.219 | −0.266 | 0.037 | −0.397 | 0.306 | −0.215 | 0.345 | 0.484 |
| SDC3 | −0.6005 | −0.2325 | −0.3475 | −0.3275 | −0.4115 | −0.1845 | −0.8275 | −0.4695 | 0.4315 | −1.2415 |
| SDCCAG0 | −0.0525 | −0.0055 | 0.0595 | −0.1425 | 0.0155 | 0.0685 | 0.5205 | −0.2875 | −0.4205 | −0.1485 |
| SEC61G | −0.401 | 0.035 | 0.164 | −0.789 | −0.211 | −0.201 | −0.331 | −0.379 | 0.019 | −0.348 |
| SECP43 | 0.8065 | 0.1725 | −0.3755 | −0.2315 | −0.0625 | 2.2615 | 1.0235 | 0.9195 | 1.4585 | 1.8845 |
| SEDLP | 1.062 | −0.791 | 1.623 | 1.454 | 0.891 | 1.331 | 0.201 | −1.709 | −0.879 | −1.128 |
| SEMA3B | 0.1395 | −0.5005 | −0.7055 | 0.0225 | −0.0955 | 0.1595 | 0.2765 | −0.3825 | 0.3235 | −0.3005 |
| SEMA3C | −0.0445 | 0.1785 | −0.2735 | 0.6925 | −0.4775 | −0.5925 | −0.3615 | −0.4755 | 1.1135 | 0.3235 |
| SEPW1 | 0.355 | −1.138 | 1.039 | 0.586 | 1.283 | 1.902 | 0.919 | −1.06 | 1.206 | 1.641 |
| SERP1 | 1.0455 | −0.2205 | 0.1335 | 0.5155 | 0.8615 | 0.1285 | 0.1175 | −0.4135 | 0.6045 | 0.0745 |
| SERPINA [symbol] | 0.907 | 0.115 | 0.531 | 3.452 | 2.918 | 1.892 | 1.797 | −1.876 | −1.648 | −1.097 |
| SERPINB [symbol] | 0.47 | −0.021 | −0.023 | 0.238 | 0.344 | −0.111 | −0.174 | 0.243 | −0.143 | 0.549 |
| SFRP1 | 0.728 | −0.141 | 1.002 | −0.054 | −0.412 | 0.233 | −0.061 | −0.528 | −0.673 | 0.786 |
| SFRS6 | 0.4645 | −0.1985 | 0.6385 | 0.7005 | 0.5965 | 0.0185 | −0.2165 | −1.4555 | −1.8995 | −0.9555 |
| SGCD | 0.9215 | 0.8565 | 2.0835 | 0.8505 | 0.0465 | −0.3515 | 1.1315 | 0.5895 | 1.9995 | 0.9525 |
| SGCE | −0.563 | −0.883 | −0.225 | −0.91 | −0.707 | −0.322 | −0.609 | −0.188 | 0.112 | −0.414 |
| SH3BGRL | 0.049 | −0.356 | −0.344 | −0.095 | 1.044 | −0.015 | 1.213 | 0.025 | −1.018 | 0.275 |
| SHFM1 | −0.2015 | −0.0615 | 0.1215 | −0.0725 | 0.0915 | −0.0235 | 0.0765 | −0.0595 | 0.3725 | −0.4735 |
| SIAT7B | −0.242 | −0.523 | 0.366 | −0.134 | −0.33 | −0.961 | −0.783 | −0.038 | −1.235 | −0.87 |
| SIGLEC12 | −0.317 | −0.024 | −0.251 | −0.144 | 0.1 | −0.048 | 0.021 | −0.12 | 0.515 | 0.395 |
| SIT | 0.0285 | 0.1165 | 0.3325 | 0.2235 | 0.0105 | −0.1995 | −0.1185 | 0.4215 | 0.2735 | 0.0085 |
| SITPEC | −0.0705 | 0.1595 | −0.1215 | −0.0005 | 0.0675 | 0.2025 | 0.1975 | 0.4955 | 0.0195 | −0.2285 |
| SKP1A | −0.5685 | 0.3305 | 0.0185 | −0.1555 | −0.3165 | −0.0055 | −0.0865 | 1.1305 | −0.3315 | −1.4545 |
| SLC12A9 | 1.387 | 1.76 | −0.993 | −0.384 | 0.576 | 1.442 | 1.482 | 2.81 | 1.155 | 1.848 |
| SLC13A3 | 2.5505 | 0.2375 | 0.8815 | −0.1235 | −0.0505 | 0.6825 | 1.0085 | −0.0355 | 1.1795 | 1.7865 |
| SLC16A6 | −0.089 | 2.509 | 0.342 | −0.109 | 0.771 | −0.381 | 1.264 | 6.487 | −0.371 | −1.191 |
| SLC19A2 | 0.2815 | 0.8245 | 0.7205 | 0.2775 | −0.3745 | 0.8485 | −1.1105 | 0.6795 | 3.7025 | 2.7105 |
| SLC1A1 | 1.2445 | −1.3085 | 0.4175 | −0.1305 | 0.1975 | −0.7845 | −0.9095 | −2.0955 | −1.6045 | −0.8045 |
| SLC1A2 | −0.5495 | −0.1735 | −0.3575 | −0.1055 | 0.1295 | 1.3165 | 0.9555 | 0.6485 | −0.7005 | −0.4855 |
| SLC24A3 | 0.069 | −0.508 | −0.881 | 0.183 | 0.119 | −0.189 | −0.1 | −0.019 | 0.817 | 0.889 |
| SLC25A29 | −0.373 | −0.232 | 0.051 | −0.465 | 0.002 | −0.685 | −0.406 | −0.062 | −0.108 | −0.332 |
| SLC25A4 | 1.41 | 0.51 | 1.732 | 0.944 | 0.626 | −0.709 | 0.885 | 0.155 | 2.356 | 0.516 |
| SLC25A5 | 0.4205 | −0.0845 | 0.0905 | 0.1605 | −0.2735 | 0.3465 | −0.2315 | 0.7995 | −1.0425 | −0.4545 |
| SLC27A2 | −0.059 | 1.411 | 0.584 | −0.06 | 0.109 | −0.459 | −0.194 | −0.04 | 0.28 | −0.073 |
| SLC27A5 | 0.403 | −0.596 | −0.156 | −0.356 | −0.993 | −1.038 | 0.014 | 0.088 | −0.721 | −0.255 |
| SLC30A8 | 3.7445 | 1.1625 | 1.4925 | 0.0735 | 0.2395 | 1.0585 | 0.8545 | −0.2245 | 4.2025 | 1.6175 |
| SLC39A4 | 0.12 | 0.583 | 2.24 | 1.184 | 1.073 | 0.693 | 0.995 | 3.266 | −1.53 | −1.979 |
| SLC39A6 | −0.0145 | 0.0445 | 0.3585 | 0.1425 | 0.1925 | 0.3645 | 0.1135 | −0.5285 | −0.5475 | −0.0435 |
| SLC40A1 | −0.8545 | −1.1785 | 0.3055 | −1.0555 | 0.7575 | −0.3595 | −0.4805 | −1.0715 | −1.2775 | −1.3665 |
| SLC41A3 | −0.405 | 0.492 | 3.029 | 0.014 | −0.193 | −0.284 | 2.657 | 0.673 | −0.354 | 0.049 |
| SLC5A6 | 1.5725 | −1.3135 | −0.5095 | −0.2675 | 1.0226 | 0.2845 | 0.6735 | −0.4355 | 1.3595 | 0.9475 |
| SLC6A4 | −0.5445 | 4.2345 | 2.5165 | 1.5135 | 0.1215 | 1.2355 | 0.7365 | 3.6575 | −0.4095 | 0.3845 |
| SLC9A3R1 | 1.3885 | 1.3115 | 1.7675 | 0.0855 | 0.3275 | 0.0015 | 0.0145 | 1.6855 | 2.5195 | 0.8365 |
| SLITRK6 | 0.434 | −0.248 | 0.035 | −0.256 | −0.374 | −0.724 | −0.555 | −0.383 | −0.067 | −0.017 |
| SMA3 | 0.143 | −0.109 | 0.428 | 0.083 | 0.413 | 0.4 | 0.249 | 0.214 | −0.952 | −0.615 |
| SMAD2 | 0.019 | −0.566 | 0.004 | −0.19 | −0.428 | −0.818 | −0.355 | 0.209 | −0.146 | 0.115 |
| SMO | 0.028 | 0.164 | 0.062 | −0.087 | 0.03 | 0.177 | −0.048 | −0.12 | −0.279 | 1.072 |
| SMU1 | 0.921 | −0.228 | −0.1 | −0.082 | −0.327 | 0.128 | −0.142 | −0.239 | −0.232 | −0.25 |
| SNPH | −0.612 | −0.159 | −0.179 | −0.756 | −0.197 | −0.283 | −0.041 | 0.179 | −0.258 | −0.52 |
| SNRPB2 | −0.3885 | −0.2555 | 0.0455 | −0.5525 | 0.0965 | −0.5115 | 0.6285 | 0.2945 | −0.1255 | −0.1285 |
| SNRPD1 | −0.56 | −0.474 | −0.322 | −0.452 | −0.444 | −0.443 | −0.53 | −0.158 | 0.222 | −0.211 |
| SNRPD3 | 0.4955 | 0.2035 | 0.1625 | 0.3865 | 0.0165 | −0.4225 | 0.3065 | −0.1445 | 0.6075 | −0.9985 |
| SNRPG | −0.0965 | −0.2175 | −0.4755 | −0.9385 | −0.3465 | −0.8995 | −0.8455 | −0.5555 | −0.3075 | −0.7975 |
| SNX7 | −0.1565 | 0.2575 | 0.2585 | 0.1055 | 0.1035 | −0.0675 | 0.5265 | 0.6725 | 1.2355 | 0.7755 |
| SO02 | −0.8695 | −0.4875 | −0.5245 | −0.3895 | 0.2116 | 0.0825 | 0.7235 | 0.6255 | −0.0645 | −0.3455 |
| SORCS1 | 0.406 | −1.358 | −1.371 | 0.4 | 0.4 | −0.977 | −0.636 | −2.862 | 0.492 | −2.266 |
| SOX4 | 0.6275 | 1.6735 | −0.8705 | 0.6795 | 0.4555 | −1.2145 | 0.0155 | 2.6885 | −1.0355 | 1.7025 |
| SOX9 | −1.327 | −1.061 | −0.992 | −1.814 | −0.139 | −0.045 | 0.595 | −0.989 | 0.113 | −0.129 |
| SPAG18 | 0.0855 | −0.0405 | 0.4295 | −0.1155 | 0.1865 | −0.5375 | −0.3065 | −0.2895 | 0.2075 | −0.3285 |
| SPAG5 | −0.0455 | −0.3345 | −0.1785 | 0.0445 | 0.2335 | −0.0585 | 0.2755 | 0.3415 | −0.0245 | 0.7825 |
| SPC18 | 0.822 | 0.897 | −0.431 | 0.236 | 1.005 | 0.165 | 0.026 | 0.171 | 0.879 | 0.504 |
| SPFH2 | −0.055 | −0.142 | −0.216 | −0.327 | −0.339 | −0.004 | 0.186 | 0.269 | 0.061 | 0.106 |
| SPG3A | 0.697 | −0.985 | −0.605 | −1.118 | −0.705 | −1.455 | −1.205 | −0.379 | −0.625 | −0.267 |
| SPPL3 | −0.04 | 0.192 | −0.026 | −0.456 | −0.176 | −0.133 | −0.123 | 0.078 | −0.346 | 0.181 |
| SQLE | 0.165 | 0.241 | 0.948 | 0.674 | −0.564 | 0.797 | 0.657 | 2.384 | 1.718 | 0.603 |
| SQRDL | 0.7565 | 1.7425 | 0.3075 | 0.2195 | 0.2895 | −0.3995 | −0.2155 | 0.1985 | −0.1665 | 0.1025 |
| SREBF1 | −0.5345 | −0.1785 | 0.0555 | −0.5135 | −0.0755 | −0.2965 | −0.5135 | −0.5075 | −0.2155 | −0.0255 |
| SRI | 0.8475 | 0.9155 | 0.3755 | 0.9325 | 0.3885 | 0.0205 | 0.1685 | −0.4075 | 3.7465 | 3.8075 |
| SRPK1 | −0.2235 | −0.5495 | 0.5895 | −0.1895 | 0.2375 | −0.3275 | −0.4285 | −0.5955 | 0.8045 | −0.1575 |
| SSFA2 | 0.1835 | −0.2585 | −0.0985 | −0.3225 | −0.5395 | −0.1095 | −0.5045 | −0.3995 | 0.2165 | 0.0185 |
| SSR4 | −0.0885 | −0.0545 | 0.0585 | 0.1375 | 0.4405 | 0.0975 | 0.2975 | −0.4765 | 0.3295 | −0.4675 |
| STARD3NI | −0.2385 | −0.1345 | −0.0135 | −0.9475 | −0.4305 | −1.3525 | −0.8745 | −0.3525 | −0.8015 | −0.5545 |
| STARD7 | 2.1955 | 1.6655 | 0.6175 | 1.8845 | 0.4845 | 1.2665 | 1.3605 | 2.7185 | −1.7125 | −0.6255 |
| STAT1 | 0.0476 | 0.1105 | −0.1945 | −0.1885 | 0.2605 | 0.0885 | 0.0295 | −0.0245 | −0.2875 | −0.8065 |
| STC2 | −0.7815 | −0.7285 | −0.3865 | −0.8585 | −0.5085 | −0.1465 | −0.0625 | −0.3175 | −0.1955 | −0.2585 |
| STK24 | −1.3915 | −0.6525 | −1.0905 | −1.9705 | −0.8275 | −1.3375 | 0.1425 | 0.8745 | −0.3975 | 0.0435 |
| STK6 | −0.003 | 1.381 | −0.114 | −0.18 | −0.071 | 0.085 | −0.322 | 0.742 | 1.821 | 0.886 |
| STMN1 | 0.1005 | −0.2415 | −0.2765 | −0.0965 | 0.0035 | −0.2255 | −0.6715 | −0.0035 | −0.9845 | 0.5725 |
| STX1A | 0.0375 | −0.8385 | −0.1995 | −0.5235 | −0.5455 | −1.3085 | −0.8565 | 0.4075 | 0.2965 | −0.0645 |
| STX3A | −0.4575 | 0.4225 | −0.3215 | −0.3795 | 0.2725 | −0.2125 | 0.2935 | 0.1885 | −0.0565 | −0.6705 |
| STYX | 0.2345 | −0.0865 | −0.0445 | 0.0075 | 0.1055 | 0.4205 | −0.1855 | 0.0255 | 0.3895 | 0.1805 |
| SUGT1 | 0.07 | 0.019 | −0.024 | −0.475 | −0.065 | −0.826 | 0.207 | −0.154 | −0.071 | 0.195 |
| SUMF1 | −0.2895 | −0.4135 | −0.6425 | −0.1465 | −0.2735 | −1.0815 | −0.6205 | −0.4415 | −0.0395 | −0.2915 |
| SUMO2 | 0.0025 | −1.0635 | 0.2965 | −0.4655 | −0.9125 | −0.3945 | −1.2015 | −0.1825 | 0.1665 | −0.0765 |
| SUV39H2 | −1.0955 | −0.7615 | −0.8615 | −1.0225 | −1.9495 | 0.9965 | 0.5965 | 0.4316 | −0.9265 | −0.1835 |
| SYAP1 | 0.8085 | −0.6185 | −0.1405 | 0.7075 | 0.7155 | 1.0015 | −0.3105 | −0.5435 | −0.2025 | −0.7945 |
| SYCP2 | 0.751 | 2.463 | −0.208 | 1.258 | 1.344 | 1.28 | 0.401 | 1.367 | −0.268 | 1.676 |
| SYNGR1 | −0.1845 | 0.2205 | 0.1335 | 0.2275 | 0.1095 | −0.1445 | −0.0435 | −0.8895 | 0.3925 | 0.2565 |
| SYTL2 | 0.0285 | −0.4105 | −0.2955 | −0.1386 | −0.0385 | 0.1805 | 0.0295 | −0.2785 | −0.1265 | 0.0305 |
| TAF18 | 0.5015 | 0.1735 | −0.4785 | −0.1735 | −0.3145 | −0.1525 | 1.4265 | −1.2915 | −0.1015 | 0.5375 |
| TAF8 | −0.665 | −0.126 | 0.12 | −0.789 | 0.284 | −0.509 | −0.195 | −0.434 | −0.002 | −0.102 |
| TARBP1 | 0.005 | −0.351 | −0.058 | −0.295 | −0.673 | −1.105 | −0.914 | −0.495 | −0.016 | −0.415 |
| TARS | 0.5905 | 0.7016 | 0.6685 | −0.2355 | 0.1485 | 1.4415 | 0.4265 | 0.4675 | −0.3985 | 0.0265 |
| TATDN1 | −0.0805 | 0.1635 | −0.3515 | 0.1755 | 0.1015 | −0.3585 | 0.0825 | −0.5885 | −0.9145 | 0.6105 |
| TBC1D12 | 0.3845 | 0.6875 | 0.1335 | −0.0175 | −0.2145 | 0.7325 | 0.6655 | 0.7375 | 0.0685 | 1.1945 |
| TBC1D14 | −0.491 | −0.069 | −0.048 | −0.156 | −0.041 | 0.209 | −0.133 | −0.08 | 0.527 | −0.04 |
| TBC1D3 | −1.1755 | 0.3976 | 0.9465 | 0.3355 | −0.0165 | 0.8516 | 2.0995 | −0.5655 | −0.9785 | −0.0095 |
| TBCA | 0.588 | 1.415 | 0.197 | 0.278 | 1.288 | 1.223 | 2.25 | 1.434 | −1.618 | −1.501 |
| TBX1 | −0.2725 | 0.1215 | −0.1925 | −0.1695 | 1.3045 | 0.6535 | 0.0265 | 0.5075 | −0.1595 | −0.2415 |
| TBX3 | 0.902 | 1.373 | 0.095 | 0.637 | 0.133 | 1.275 | 0.057 | 2.318 | 0.67 | 1.84 |
| TCAP | 0.931 | 0.994 | −0.635 | 0.503 | 0.008 | 1.718 | 0.196 | 0.848 | 0.142 | 1.083 |
| TCEAL1 | 0.7545 | 1.3895 | −0.1875 | 0.9875 | −0.2005 | 1.9455 | 0.1905 | 2.6915 | −0.2735 | 1.0395 |
| TCEAL3 | −0.488 | −0.112 | −1.398 | −1.676 | −0.649 | 1.434 | 1.655 | −0.197 | 0.238 | 0.42 |
| TCEAL4 | −0.477 | −0.442 | −0.079 | −0.478 | 0.108 | −0.238 | −0.364 | −0.141 | −0.599 | −0.412 |
| TCF15 | 0.158 | −0.276 | 0.305 | 0.776 | 1.106 | 0.616 | 0.384 | −0.483 | −1.848 | −1.512 |
| TCF3 | −0.445 | 0.037 | 0.277 | −0.33 | −0.543 | −0.278 | −0.229 | −0.019 | −0.006 | 0.347 |
| TCF7L2 | 0.3805 | −0.1765 | 0.0845 | 0.3965 | 0.2685 | 0.2975 | 0.2685 | −0.1015 | −0.4845 | 0.0465 |
| TDRD7 | −1.0715 | −0.6655 | −0.2005 | 1.0025 | 1.4205 | 0.2405 | 1.0835 | 1.3525 | −4.8405 | −3.2645 |
| TESK2 | −0.402 | −0.559 | −0.824 | 1.071 | 0.368 | 1.211 | 1.02 | −0.794 | 0.471 | −0.055 |
| TFAP2B | −1.171 | −0.119 | 0.051 | 0.775 | 0.198 | −0.786 | −0.391 | −0.858 | −0.565 | −0.791 |
| TFCP2L1 | −0.2345 | −0.4455 | −0.4395 | −0.5975 | 1.2755 | 0.6875 | 2.0145 | 0.9575 | 4.5155 | 3.7095 |
| TFCP2L2 | 0.5595 | −0.3875 | −1.9815 | −0.3365 | 2.4915 | −0.0125 | 1.7225 | 0.2295 | 3.6335 | 1.7525 |
| TFF1 | −0.1465 | −0.4355 | 0.5535 | −0.5865 | 0.8035 | −0.1805 | −0.4295 | 0.1475 | 0.1535 | −0.7115 |
| TFF3 | 0.068 | 0.845 | −0.01 | 1.069 | 1.712 | 0.622 | 1.092 | 0.886 | 0.367 | 1.843 |
| TFRC | 0.0245 | 0.1235 | 0.1385 | 0.2065 | 0.2465 | −0.3645 | 0.0255 | 0.1815 | −0.5315 | 0.0805 |
| TGFBR3 | −0.829 | −0.472 | −0.248 | −0.278 | −0.43 | 0.301 | 0.323 | −0.494 | −0.65 | −0.622 |
| THY28 | −0.2585 | 0.6125 | 0.3985 | 0.7945 | 0.6355 | 0.3695 | 0.2315 | −0.1225 | 0.4035 | −0.2105 |
| TIF1 | −0.007 | 0.281 | 0.565 | −0.09 | −0.363 | −0.432 | −0.568 | −0.076 | 0.679 | 0.569 |
| TIGA1 | −0.977 | −1.111 | −0.345 | −0.975 | −0.691 | −0.44 | 0.245 | −0.172 | −0.276 | −0.078 |
| TIM14 | −0.3225 | −0.0865 | −0.4885 | 0.1225 | 0.0185 | −0.2145 | −0.1055 | 0.0905 | 0.3925 | −0.1235 |
| TIMELES [symbol] | −0.4025 | 0.3015 | 0.5685 | 0.1825 | 0.0915 | −0.4245 | −0.1155 | −0.3485 | 0.1815 | −0.3275 |
| TIMM9 | −0.065 | 0.306 | 0.202 | 0.422 | −0.235 | −0.094 | −0.592 | 1.011 | −0.18 | 1.443 |
| TINP1 | −0.8595 | −1.0445 | −0.8775 | −1.0695 | −0.8405 | −1.9855 | 0.2275 | 0.4226 | 0.7345 | −0.0945 |
| [symbol] IP1208 | −0.419 | −0.21 | −0.257 | 1.137 | 0.38 | 0.662 | 0.365 | −0.84 | −1.77 | −0.6 |
| TK1 | −0.438 | 0.04 | −0.599 | −0.432 | −0.055 | 0.456 | 0.633 | −1.227 | 0.441 | −0.493 |
| TLE2 | −0.73 | −0.506 | 0.259 | 0.714 | −0.814 | −0.708 | −0.443 | 0.441 | −0.925 | 0.503 |
| TLR5 | −0.181 | 0.644 | 0.022 | −0.667 | −0.5 | −0.064 | 0.098 | −0.278 | 0.594 | 0.14 |
| TM4SF12 | 0.16 | 0.106 | 0.361 | −0.448 | −0.691 | −0.803 | −0.599 | −0.653 | 1.034 | 0.504 |
| TM4SF13 | 0.653 | 0.664 | 0.751 | 0.248 | 0.346 | 0.058 | −0.148 | −0.387 | 0.809 | 1.633 |
| TMEM14A | −0.7015 | −0.1675 | −0.1295 | −0.1345 | 0.0915 | −0.3125 | 0.0385 | −0.2395 | 0.5275 | −0.0105 |
| TMEM25 | −0.1975 | 0.6135 | 0.3255 | 0.3625 | 0.4065 | 0.3335 | 0.8555 | −0.6115 | −0.9975 | 0.0185 |
| TMEM4 | −0.391 | −0.079 | 0.055 | −1.067 | 0.11 | −0.346 | −0.269 | −0.908 | −0.415 | −0.657 |
| TMPRSS5 | 0.01 | −0.78 | −1.478 | 0.3 | −0.414 | −0.765 | −0.531 | −0.62 | −1.769 | 0.24 |
| TMSB10 | −0.041 | −1.553 | 0.962 | 0.417 | −0.051 | −1.1 | −0.065 | −1.201 | −3.087 | −0.818 |
| TMSNB | −0.6985 | −0.7415 | 0.3445 | −0.3075 | 0.2135 | −0.8445 | −0.5295 | −0.2095 | −1.4805 | 0.1365 |
| TNFRSF1 [symbol] | −1.159 | −0.891 | −0.508 | −0.242 | 0.924 | −0.788 | −0.186 | −0.558 | −0.763 | 0.168 |
| TNFRSF2 [symbol] | 0.4205 | −0.3445 | 0.8345 | −1.0155 | −1.4335 | 0.0195 | −0.6435 | −0.1095 | −0.6765 | 0.5315 |
| TNFRSF2 [symbol] | −0.2545 | 0.3815 | 3.2705 | 3.0225 | 3.9485 | 1.7615 | 0.4605 | −0.2415 | −1.1385 | −0.1345 |
| TNFSF4 | −0.1055 | 1.0715 | −0.1925 | 0.3535 | 0.1435 | 1.0525 | 0.2185 | −0.6405 | 0.6585 | 0.1605 |
| TNFSF7 | −0.5125 | −0.0315 | −0.4755 | −0.0395 | 0.9965 | 1.3795 | 0.4865 | 0.0355 | 1.2845 | −0.0365 |
| TNIK | 1.1785 | −0.4315 | 0.6425 | 1.7925 | 1.2465 | 1.2995 | 0.4135 | −0.7535 | −0.8215 | −0.6255 |
| TNNC2 | −0.339 | −0.662 | 0.876 | 0.051 | −0.829 | −0.415 | −1.03 | 2.311 | −1.137 | −0.115 |
| TNNI2 | −0.013 | −0.254 | 0.275 | −0.145 | 1.474 | 0.97 | 0.386 | 0.626 | 0.437 | 0.351 |
| TNNI3 | 0.2865 | 0.0915 | −0.0985 | 0.1535 | −0.5255 | −0.2845 | 0.5985 | −0.1895 | 0.8985 | 0.2675 |
| TOB1 | −0.1405 | 0.2555 | 0.0725 | 0.0425 | 0.4755 | 0.0355 | 0.3735 | −0.6195 | 0.1905 | −0.3125 |
| TOM1L1 | −0.8025 | 0.2145 | 0.0065 | −0.6065 | −0.1665 | −0.3235 | 0.0895 | 0.0335 | −0.8185 | −1.1395 |
| TOMM7 | −0.9585 | −0.7715 | −0.6485 | −1.1435 | −0.7475 | −0.1765 | 0.5885 | −0.4975 | 0.7605 | 0.3055 |
| TOP1MT | −0.037 | 0.314 | 0.122 | 0.499 | 0.198 | −0.049 | 0.313 | 0.278 | −0.14 | −0.011 |
| TOP2A | −0.5845 | −0.1485 | −0.2505 | −0.4895 | −0.4865 | −0.1905 | 0.0935 | −0.3225 | 0.0375 | −0.2615 |
| TOP2B | −1.384 | −0.788 | −0.951 | −1.499 | −0.827 | −1.398 | 0.343 | −1.254 | 0.67 | 0.329 |
| TOPBP1 | −0.0145 | −0.9015 | 0.2185 | −0.1275 | 0.3675 | 0.9645 | 0.3275 | 1.2845 | 0.2275 | −0.8015 |
| TOPK | −0.0145 | −1.1795 | −0.1555 | −0.4025 | −1.0685 | −1.9655 | −0.2395 | −0.7395 | 0.2615 | 0.0275 |
| TP53AP1 | 0.3265 | 0.3455 | 0.2255 | −0.1855 | 0.3915 | 0.5295 | 0.6495 | −0.4705 | −0.3285 | −1.0975 |
| TP53BP2 | 0.0865 | −0.9965 | 0.1005 | 0.5785 | 0.4615 | 0.4915 | −0.3605 | −0.9815 | −2.5245 | −2.6475 |
| TP53INP1 | −0.0615 | 0.0005 | 0.0865 | −0.4635 | −0.2216 | −1.0615 | −0.6425 | −0.3085 | −0.1305 | 0.2715 |
| TPD52L1 | −0.156 | 0.209 | 0.319 | −0.469 | −0.065 | −1.15 | −0.045 | −0.472 | 0.907 | −1.171 |
| TPRT | 0.0285 | −0.3675 | −0.7855 | −0.4805 | 0.1875 | 0.4995 | 0.4325 | −0.6335 | 0.0135 | −0.2415 |
| TRA@ | 0.417 | 0.269 | 0.691 | 0.056 | 0.253 | −0.294 | 0.253 | 0.765 | −0.5 | 0.838 |
| TRAF4 | −0.042 | 3.669 | −0.275 | 0.886 | 0.097 | 0.095 | 0.397 | 4.837 | 2.866 | 0.009 |
| TRAPPC4 | 1.371 | 0.382 | −0.869 | 0.124 | 0.364 | 0.675 | 0.078 | 1.095 | −0.222 | 0.344 |
| TRH | −0.5245 | −0.0705 | −0.5745 | 1.0185 | 0.7235 | 0.1755 | 0.1245 | −0.5125 | 0.1945 | 0.3425 |
| TRIM17 | 0.2625 | −0.6415 | 0.2895 | 0.3695 | 0.0855 | 0.2725 | −0.3025 | −0.4425 | −0.4355 | 0.2815 |
| TRIM2 | 0.2895 | 0.1595 | −0.0945 | 0.0205 | 0.0095 | −0.1205 | 0.1615 | −0.2195 | 0.7555 | 0.4085 |
| TRIM6 | 0.543 | 0.87 | 0.052 | −0.103 | −0.723 | −0.663 | 0.6 | 0.668 | 0.83 | 0.037 |
| TRIP3 | 0.0355 | 0.1755 | −0.6405 | −0.3575 | −0.0945 | −0.2205 | −0.3885 | −0.0025 | 0.6635 | 0.4365 |
| TRPS1 | 0.5525 | 0.3635 | 0.1905 | 0.3625 | 0.5425 | 0.4195 | 0.4645 | 0.4485 | 0.3735 | 0.3555 |
| TSG101 | 0.0965 | −0.3175 | 0.4315 | 0.2105 | 0.9675 | 0.4755 | 0.7085 | 0.8285 | 0.3345 | −0.2035 |
| TTC10 | −1.0945 | −0.7485 | 0.1845 | −0.8975 | −0.4805 | −1.1195 | −0.3175 | −0.9135 | 0.0285 | −0.6835 |
| TTC12 | −0.183 | −0.254 | 0.112 | −0.214 | 0.241 | −0.184 | 0.012 | −0.268 | −0.385 | 0.073 |
| TTK | −0.3055 | −0.3465 | −0.1675 | 1.0225 | 1.0065 | −0.3125 | 0.1455 | −0.3115 | −0.5585 | −0.2645 |
| TTLL4 | −0.097 | 0.868 | −0.225 | −0.088 | 0.16 | −0.118 | −0.007 | 1.087 | 1.487 | 0.896 |
| TTYH1 | 0.0085 | 0.1555 | −0.4345 | −0.0595 | 0.6745 | 0.7955 | 1.2245 | 0.7235 | −0.1075 | −0.0295 |
| TUBB3 | −1.0185 | −0.2445 | −0.2005 | −0.4775 | −0.2075 | −0.2375 | −0.3905 | 0.0415 | 0.8525 | −0.5405 |
| TULP3 | −0.764 | −0.375 | −1.095 | −1.694 | −0.085 | −0.885 | 0.385 | −0.189 | −0.175 | −0.234 |
| TXN | 0.043 | 0.196 | −0.289 | −0.403 | −0.111 | 0.425 | −0.015 | 0.344 | 0.105 | −0.186 |
| TYMS | −0.2225 | −0.4545 | −0.0175 | 0.6045 | −0.1765 | −0.3095 | −0.5465 | −1.6795 | −1.5245 | −1.3715 |
| U5-116KD | −0.2815 | −0.4375 | 0.3005 | −0.4915 | −0.6945 | −0.2875 | −0.2065 | −0.0855 | 0.2255 | −0.2335 |
| UBE2E3 | −0.108 | −0.001 | −0.181 | −0.164 | 0.179 | −0.078 | 0.373 | −0.021 | −0.201 | 0.042 |
| UBL5 | 0.2865 | 1.7525 | −0.1025 | −0.0985 | −1.0985 | 0.8455 | −0.0865 | −0.6305 | −1.0595 | −0.2765 |
| UBN1 | −0.157 | −0.056 | 0.281 | 0.251 | 0.007 | −0.613 | −0.707 | −0.249 | 0.504 | −0.906 |
| UCHL1 | −0.194 | 2.545 | 0.22 | −1.41 | −0.075 | 1.336 | 0.926 | 1.532 | 0.017 | 0.356 |
| UCHL3 | −0.745 | −0.658 | 0.115 | −1.245 | −0.306 | −0.81 | −1.262 | −0.65 | −0.231 | −0.141 |
| UGCG | −0.0485 | 1.2105 | 1.8265 | 6.2955 | 5.2685 | 3.1085 | −0.5655 | 2.5915 | 0.7645 | −0.3715 |
| UGCGL1 | −0.2925 | −0.5245 | −0.5035 | −0.2945 | 0.0735 | 0.4165 | 0.1295 | 0.0235 | −0.8945 | −1.3145 |
| UGT2B7 | 0.1015 | −0.2845 | −0.1255 | −0.5125 | 0.0455 | −0.2435 | 0.1545 | 0.1615 | −0.3425 | −0.5775 |
| UNC13D | 0.022 | 0.562 | 0.767 | 0.528 | 0.089 | 0.454 | −0.274 | 0.515 | 1.163 | 0.253 |
| VAMP8 | 0.497 | 0.379 | −0.021 | −0.041 | −0.534 | −0.268 | −0.374 | −0.17 | 0.093 | −0.729 |
| VAPA | 0.7275 | 0.3035 | 1.2355 | 0.0085 | 0.0125 | −0.9755 | 0.4815 | 0.5715 | −0.2505 | −0.2035 |
| VAV1 | −0.563 | 0.046 | 1.316 | 0.224 | 0.323 | 0.953 | 0.673 | 0.654 | 0.124 | −0.868 |
| VAV3 | 0.039 | −0.239 | 0.068 | −0.283 | −0.173 | −0.699 | −0.361 | −0.136 | −0.098 | 0.218 |
| VCX3 | −0.043 | 0.115 | 1.287 | 0.053 | 0.179 | −0.27 | −0.811 | −0.1 | −0.044 | 0.589 |
| VDAC2 | −0.2525 | −0.2315 | −0.4515 | 0.2375 | −0.1315 | −0.1815 | −0.2515 | −0.4005 | 0.0185 | −0.3805 |
| VEST1 | −0.132 | −0.037 | 0.399 | −0.004 | 0.248 | −0.045 | 0.286 | −0.183 | 0.092 | −0.093 |
| VGLL1 | −0.1385 | 0.3135 | −0.5955 | 1.0975 | 0.9835 | 0.7225 | 0.7305 | −0.3255 | 0.0365 | −0.2085 |
| VMD2L2 | 0.414 | 0.071 | 0.068 | −0.407 | −0.129 | −0.188 | −0.059 | 0.284 | 0.474 | 0.235 |
| VSX1 | 0.283 | −0.129 | −0.243 | −0.331 | −0.11 | −0.199 | −0.011 | −0.608 | 0.711 | 0.307 |
| WARP | 0.061 | 0.235 | −0.058 | −0.159 | 0.284 | 0.677 | 0.891 | 0.484 | −0.205 | 0.5 |
| WDR26 | −0.3595 | 0.4495 | −0.1305 | 0.0845 | 0.0365 | −0.0075 | 0.3115 | 0.3825 | 0.1225 | 0.1915 |
| WDR6 | 0.601 | 1.206 | −0.435 | −0.326 | −0.635 | 1.671 | 1.024 | 1.995 | −0.227 | 0.901 |
| WHSC1L1 | 0.091 | −0.399 | −0.259 | 0.565 | 0.951 | −0.269 | 0.161 | −0.193 | −0.499 | 0.023 |
| WNT3 | −0.1765 | 0.1295 | 0.2355 | −0.3985 | 0.2355 | −0.5305 | −0.1065 | −0.7425 | −0.0105 | 0.2005 |
| WNT6 | 1.0835 | 0.6165 | 0.3195 | 0.1805 | 0.0985 | 1.3535 | 0.9295 | 0.7655 | −0.1985 | 0.4425 |
| WSB2 | 0.0285 | 0.0155 | −0.0975 | 0.5425 | 0.1945 | 0.2305 | −0.1795 | 0.2416 | 0.1705 | −0.5415 |
| WWP1 | 1.2795 | 0.6545 | 0.4715 | 0.2675 | 0.2175 | −0.2415 | 0.8645 | 1.2265 | 1.9425 | 1.3576 |
| WWP2 | −0.018 | −0.234 | −0.383 | −0.196 | 1.178 | 0.039 | 0.735 | −0.005 | 0.005 | −0.452 |
| XBP1 | −0.403 | −0.537 | −0.615 | −0.093 | −0.352 | 0.342 | 0.325 | 0.472 | −0.533 | −0.4 |
| XYLT2 | 0.0975 | 0.1295 | −0.2965 | −0.3065 | −0.2855 | −0.5885 | −0.7945 | −0.0465 | 1.4955 | 0.0455 |
| YBX2 | 0.67 | −0.138 | −0.099 | 0.067 | −0.334 | −0.365 | −0.223 | 0.41 | 0.259 | 0.477 |
| YIF1 | −0.0945 | −0.0945 | 0.1405 | 0.0455 | 0.3015 | 0.2955 | −0.2405 | 0.2425 | −0.2115 | −0.8125 |
| ZA20D3 | 0.0675 | −0.1125 | 0.1515 | −0.0485 | 0.1465 | −0.0225 | 0.1405 | 0.0335 | −2.1155 | −0.3116 |
| ZAP128 | 0.2275 | 0.9005 | −0.0145 | 0.3985 | 0.5375 | 0.8195 | 1.1075 | 0.1855 | 0.3105 | −0.1835 |
| ZBED3 | −0.198 | −0.228 | 0.212 | −0.565 | 0.059 | −0.123 | −0.095 | −0.383 | −0.414 | −0.212 |
| ZBTB4 | −0.231 | −0.251 | 0.151 | −0.257 | 0.197 | −0.13 | −0.103 | −0.363 | −0.444 | −0.504 |
| ZCWCC1 | −0.4485 | 0.0685 | −0.9625 | −0.9185 | −0.7475 | −0.5875 | −0.8395 | −0.0725 | −0.1335 | −0.0625 |
| ZCWCC2 | −0.779 | 0.638 | 0.21 | −0.056 | 0.835 | 0.288 | 0.528 | 0.33 | −0.597 | −0.494 |
| ZDHHC13 | 1.4285 | 1.0585 | 0.8045 | 0.4155 | 1.1445 | 1.2845 | 0.9545 | −1.0295 | −0.5655 | 0.1795 |
| ZDHHC14 | −0.0055 | 0.0585 | 0.2125 | 0.3605 | 0.6845 | 0.0085 | 0.6265 | −0.0315 | −0.4676 | −0.3935 |
| ZFHX4 | 0.9745 | −0.0035 | −0.0835 | 0.8855 | 0.3095 | 0.9355 | 0.5935 | −0.1625 | −0.4485 | −0.1025 |
| ZFYVE21 | −0.214 | −0.455 | −0.281 | −0.647 | −0.486 | 0.352 | −0.014 | 0.308 | 0.728 | 0.179 |
| ZNF133 | 0.382 | 0.332 | 0.063 | −0.016 | 0.052 | 0.074 | 0.112 | 0.256 | 0.253 | 0.345 |
| ZNF165 | −0.3625 | 0.1955 | 0.2295 | −0.0245 | 0.0575 | −0.0915 | 0.4395 | 0.0655 | −0.0645 | 0.0475 |
| ZNF24 | 0.169 | −0.339 | −0.197 | −0.243 | −0.141 | −0.422 | 0.253 | −0.742 | −0.17 | 0.765 |
| ZNF318 | −0.1895 | −0.0235 | 0.1345 | −0.2655 | −0.2255 | 0.0835 | −0.0095 | −0.0535 | 0.1515 | −0.0505 |
| ZNF54 | −0.9295 | −0.0435 | 0.3075 | 1.3245 | 0.8316 | 0.6705 | 0.4715 | −0.1615 | −1.4916 | −0.6985 |
| ZNF432 | 0.3795 | −0.4095 | 0.1015 | 0.0055 | −0.3215 | −0.4215 | 0.0265 | −0.6985 | −0.2925 | 0.1445 |
| ZNF482 | 0.708 | 0.005 | 0.193 | −0.462 | −0.362 | 0.984 | −0.005 | 0.78 | 1.114 | 0.594 |
| ZNF505 | 0.281 | 0.74 | 0.519 | 0.602 | 0.252 | 0.747 | 0.178 | 0.182 | −0.679 | 0.782 |
| ZNF552 | 0.1705 | −0.0005 | −0.4145 | −0.1855 | 0.1515 | 1.5595 | 0.9375 | 0.9655 | 0.5355 | 1.0175 |
| ZNF577 | −0.0735 | −0.3835 | −0.3655 | −0.4715 | −0.0575 | −0.1095 | 0.2955 | −0.0245 | −0.5455 | −0.2795 |
| ZNF587 | 0.0165 | 0.1315 | −0.0535 | 0.0665 | −0.1355 | 0.3485 | 0.2225 | −0.1155 | −0.0085 | 0.2305 |
| ZNF598 | −0.2955 | 0.0005 | 0.0805 | −0.1385 | 0.1555 | −0.0735 | 0.3875 | 0.1725 | −0.3645 | 0.1185 |
| ZNF599 | −0.025 | −0.079 | 0.055 | −0.107 | 0.114 | 0.288 | −0.33 | −0.336 | −0.69 | −0.121 |
| ZNF606 | 0.462 | −0.286 | 0.025 | 0.173 | −0.389 | −0.492 | −0.402 | −0.094 | −0.09 | 0 |
| ZNF614 | 0.351 | 0.032 | −0.376 | −0.288 | −0.042 | 0.226 | 0.396 | −0.305 | −0.125 | −0.347 |
| ZNF639 | | | | | | | | | | |
| ZNF7 | | | | | | | | | | |
| | Sample | | | | | | | | | |
| | PB479 | 9830-0000 | UB-82 | 9830-0104 | UB-81L | UB-81R | UB-99 | UB-59LN | BC00004 | BR00-038 [symbol] |
| | Class | | | | | | | | | |
| Symbol | LUMINAL | LUMINAL | LUMINAL | LUMINAL | LUMINAL | LUMINAL | LUMINAL | LUMINAL | LUMINAL | LUMINAL |
| | 2.9515 | 3.3355 | 2.2185 | 1.1115 | 2.2465 | 3.0455 | 2.2795 | 3.4195 | 3.1535 | 0.4285 |
| | 0.282 | 0.46 | −0.317 | 0.341 | 0.656 | 0.174 | 0.157 | −0.024 | 0.08 | 0.268 |
| ABAT | −0.998 | 3.339 | 2.344 | 2.646 | 3.244 | 1.345 | 1.404 | 1.24 | 1.359 | −1.431 |
| ABC1 | −0.8335 | 0.0985 | −1.3235 | 0.2375 | −0.0415 | −0.2085 | −0.6765 | −0.0505 | 0.5455 | −0.1185 |
| ABCC11 | 0.0175 | 0.3565 | −1.0345 | 1.0875 | 1.0295 | 0.9805 | 1.1275 | 0.7145 | −1.0445 | 0.5345 |
| ABCC3 | 0.403 | 0.196 | 1.104 | −0.762 | −1.299 | −0.492 | 0.807 | 0.099 | −0.531 | −0.497 |
| ACAS2L | −0.1415 | −0.6075 | −0.0485 | −0.2845 | −0.5255 | −0.4095 | −0.0795 | −0.5545 | 0.3105 | −0.3045 |
| ACAT1 | 2.317 | 2.68 | −0.034 | 1.843 | 1.224 | 2.647 | 1.067 | 2.19 | 1.74 | −0.653 |
| ACAT2 | −0.8115 | 1.0835 | 0.8545 | 1.6235 | 1.0875 | −0.1425 | 0.7705 | 0.5785 | 0.2885 | −0.4035 |
| ACOX2 | −0.6785 | 0.6345 | −0.0145 | −0.0005 | −1.1195 | 0.1785 | −0.4805 | 0.1255 | 0.0005 | −0.4405 |
| ACDX3 | 0.6375 | −0.2655 | 0.9725 | −0.3405 | −1.0895 | −0.8785 | −0.3175 | −1.0575 | 2.3795 | 1.2145 |
| ACSL3 | −0.831 | 0.671 | −0.042 | −0.267 | 0.751 | −0.052 | 0.143 | 0.269 | 0.634 | 0.654 |
| ACTA1 | −0.0045 | −0.3805 | −0.1165 | 0.1115 | −0.0055 | −0.1495 | −0.1675 | −0.3685 | −0.0825 | −0.1235 |
| ACTB | −0.4645 | −0.1275 | −0.9095 | 0.8325 | −0.2395 | −0.6575 | −1.0545 | −0.4765 | −0.2745 | −0.6665 |
| ADAM33 | 1.954 | 1.543 | −1.302 | −1.248 | 0.085 | 0.528 | −1.198 | −0.904 | −0.537 | 3.052 |
| ADAMDE [symbol] | 0.8005 | 0.4545 | 0.7305 | −0.7025 | 0.0125 | 0.7465 | 0.6605 | 1.0715 | 0.9435 | −0.7485 |
| ADPN | 0.6265 | −0.9735 | −1.2855 | 0.5685 | −0.9685 | −0.5395 | 0.0325 | −0.9775 | −0.1945 | 0.2885 |
| ADPRHL1 | 0.0665 | −0.8035 | −0.2485 | 3.2095 | 1.9085 | 1.8275 | 2.3455 | 3.1095 | 0.1875 | 1.0775 |
| ADRA2C | 3.0875 | −0.0185 | −0.0295 | −0.5395 | 0.5655 | 0.6225 | 0.3115 | 0.3955 | −0.6825 | −0.2015 |
| AGR2 | 1.6925 | 0.3055 | 0.0135 | −0.1175 | −0.1465 | 2.0655 | 0.2235 | −0.3215 | −0.1345 | 0.0225 |
| AGT | 0.759 | 0.519 | 0.1 | 0.282 | 0.622 | 1.118 | 0.198 | 0.189 | 0.258 | −0.145 |
| AGTR1 | −0.233 | −0.481 | −1.07 | −1.732 | −0.979 | 0.767 | −0.195 | −0.723 | 1.288 | 1.223 |
| AHCYL1 | −0.11 | −0.018 | 0.256 | −0.401 | 0.129 | 1.64 | 0.766 | 1.449 | 0.058 | 0.307 |
| AHNAK | 0.03 | 1.029 | 0.04 | 0.513 | −0.284 | 0.278 | 1.267 | 0.811 | −0.075 | −0.641 |
| AHR | 0.1255 | 0.2225 | 0.1615 | 1.2865 | 0.1165 | 0.3115 | 0.1395 | 0.0335 | −0.2595 | −0.3455 |
| AIM1 | −0.1385 | 0.6055 | 0.2555 | −0.9435 | −0.8185 | 0.1015 | 0.1945 | 0.6165 | −0.0475 | 0.2725 |
| AK2 | −0.2965 | 1.8525 | 3.5816 | −0.3295 | 2.7516 | 2.5985 | 1.6335 | −2.6995 | −1.0425 | −0.2415 |
| AK3L1 | −0.037 | −0.421 | 0 | −1.436 | 0.158 | 0.391 | 0.455 | 0.518 | −0.683 | −0.813 |
| AK5 | −0.207 | −0.163 | 0.04 | 0.282 | 0.396 | 0.387 | −0.191 | −0.27 | 0.148 | 0.369 |
| AKAP1 | −0.0565 | 0.1895 | 0.2735 | 1.5315 | −0.5835 | 0.0025 | −0.1695 | −0.8375 | 1.6616 | 1.1025 |
| AKR1A1 | −0.074 | 0.33 | 0.04 | −1.171 | −0.527 | 0.039 | 0.009 | 0.434 | 0.021 | −0.773 |
| AKR7A3 | 0.6065 | 0.2795 | 0.8105 | −0.0235 | 0.2425 | 0.0875 | 1.1115 | 0.9495 | 0.1765 | −0.1575 |
| ALG5 | 0.4415 | 2.3995 | −1.2435 | −0.8895 | −0.7505 | −0.1825 | −0.2015 | −0.2816 | 1.2675 | −0.0345 |
| ALG8 | −0.4485 | −0.2385 | 0.0985 | −0.7705 | 0.3515 | −0.2935 | −0.5325 | −1.0195 | −0.4705 | 0.1735 |
| AMFR | 0.243 | −0.075 | 0.003 | −0.154 | −0.2 | 0.008 | 0.298 | 0.258 | 0.163 | 0.06 |
| AMPD2 | −0.912 | −0.981 | −0.243 | −1.246 | −0.71 | −0.037 | −1.068 | −0.038 | −0.75 | 0.282 |
| ANAPC11 | −0.828 | −0.14 | −0.174 | −0.495 | 0.03 | −0.558 | −0.255 | 0.015 | 0.688 | −0.541 |
| ANKRD22 | −0.94 | −0.609 | 0.051 | −1.761 | −1.373 | −1.649 | −1.004 | −0.481 | 0.387 | −1.657 |
| ANKRD27 | 0.187 | 0.076 | 0.045 | −0.304 | −0.327 | −0.08 | −0.314 | −0.715 | 0.483 | 0.6 |
| ANLN | −0.146 | 1.377 | 0.904 | −1.615 | −1.868 | −1.898 | −0.276 | −0.832 | 1.067 | −0.251 |
| ANP32A | 1.042 | 0.922 | −0.338 | 0.812 | 0.963 | 1.185 | 1.003 | 1.355 | 1.508 | 0.522 |
| ANXA3 | −0.7355 | −0.3725 | −0.1225 | −0.3365 | −2.0635 | −1.4135 | −1.4575 | −0.4825 | −0.5505 | −1.4885 |
| APBB2 | 0.6705 | 3.9385 | 1.0525 | −1.2105 | 3.5495 | 0.9805 | 0.2295 | 1.1085 | −1.4035 | −0.6295 |
| APOBEC3 | 0.695 | 0.98 | 0.795 | 0.757 | 0.788 | 0.945 | 1.457 | 2.054 | 0.879 | −0.888 |
| AQP5 | −0.95 | 1.353 | 3.481 | −0.669 | −1.2 | −0.767 | 0.07 | −0.674 | 2.53 | −1.594 |
| AR | 0.0545 | −0.0875 | −0.0605 | −0.9315 | 0.1975 | −0.3625 | 0.1855 | 0.4315 | −0.2915 | −0.3585 |
| AREG | 0.101 | 0.035 | 0.423 | 0.993 | 0.824 | 1.141 | 0.822 | 0.889 | −0.023 | 1 |
| ARF1 | 0.5045 | 0.4545 | 0.3635 | −0.8395 | −0.7395 | −0.2475 | 0.3675 | 0.7015 | 1.6165 | 0.5105 |
| ARHGDI [symbol] | −0.352 | 0.254 | −0.426 | 0.134 | −0.15 | −0.369 | 0.007 | −0.024 | −0.095 | −0.268 |
| ARL6IP | 0.6275 | 0.6335 | 0.7465 | 1.2995 | −0.1055 | 0.5405 | 0.4395 | 0.7435 | −0.3445 | −0.4485 |
| ARL6IP2 | 1.205 | 0.768 | −1.208 | −0.522 | 2.864 | 3.267 | 0.437 | 2.542 | −1.018 | 0.472 |
| ARMCX2 | 1.276 | 0.667 | −1.053 | −0.494 | 2.69 | 2.914 | 0.471 | 2.301 | −1.16 | 0.262 |
| ARNT2 | −0.025 | −0.325 | 1.189 | −1.028 | −0.779 | 0.852 | −0.589 | −0.75 | −0.064 | −0.861 |
| ARNT2 | −0.3185 | −0.4255 | −0.3905 | −0.9515 | 0.0485 | −0.3825 | −0.2845 | −0.7075 | 0.0265 | 1.0285 |
| ARP3BET [symbol] | −0.2105 | −0.3085 | −0.2585 | −0.5805 | −0.5375 | −0.2825 | 0.0495 | 0.0695 | 0.1995 | 0.0235 |
| ARPC1B | 1.77 | −0.555 | −0.267 | −1.125 | −0.341 | −0.625 | 0.131 | −0.618 | −0.813 | 0.005 |
| ARPC5L | −0.0755 | 0.3195 | −0.3195 | 0.4345 | −0.6565 | −0.0955 | −0.3015 | 0.0855 | 0.0225 | −0.4845 |
| AS89 | −0.058 | −0.1 | 0.213 | 0.312 | −0.202 | −0.072 | 0.275 | 0.123 | 1.352 | 0.643 |
| ASF1A | −0.1075 | −0.3845 | −0.4565 | −0.6455 | −1.0295 | −1.0705 | −0.8695 | −0.7035 | −0.3965 | −1.4925 |
| ASH2L | −0.772 | −1.379 | 0.43 | −2.575 | −1.763 | −1.881 | −0.823 | −0.348 | −0.483 | −1.502 |
| ASK | −0.14 | −0.004 | −0.619 | −1.171 | −0.327 | −0.799 | 0.11 | −0.659 | 0.326 | 0.214 |
| ASPM | 0.0245 | −0.3245 | −0.1605 | −0.6225 | −1.8805 | −0.5335 | −0.6465 | 0.0055 | 0.7165 | −0.7415 |
| ASS | 0.0185 | −0.1505 | −0.0705 | 0.7815 | 0.2315 | −0.2045 | 0.5625 | −0.0265 | 0.0565 | −0.1195 |
| ATAD2 | −0.4385 | −0.1145 | −1.0515 | −0.2255 | −0.3315 | −0.8635 | −1.1095 | −1.3665 | −1.0465 | 0.3235 |
| ATF5 | −0.494 | 0.276 | 0.006 | −0.418 | −0.671 | −0.378 | 0.002 | 0.292 | 0.01 | 0.508 |
| ATF7IP2 | 0.24 | 0.087 | −0.132 | −0.393 | −0.249 | −0.262 | 0.476 | 0.58 | −0.099 | 0.019 |
| ATP2B1 | 0.484 | 0.32 | −0.143 | −0.177 | −0.425 | −0.098 | 0.298 | 0.171 | −0.093 | 0 |
| ATP5G1 | 0.1745 | 0.0885 | −0.2215 | −0.3125 | 0.1545 | 0.3785 | 0.2585 | 0.0725 | 0.8765 | 1.1175 |
| ATP5O | −0.611 | −2.348 | 0.552 | 1.125 | −0.427 | −0.35 | 2.744 | 1.877 | −0.372 | −1.12 |
| ATP6AP1 | 0.167 | 0.906 | 0.904 | −0.836 | −0.19 | 0.015 | −0.325 | −0.164 | −0.318 | 1.293 |
| ATP6V0A4 | −0.611 | 0.577 | −0.031 | −0.21 | −0.413 | 0.371 | 0.057 | 0.097 | 0.427 | 0.37 |
| ATP6V1B [symbol] | 0.2795 | −0.1105 | 0.1335 | 0.5425 | −0.0835 | 0.4385 | −0.5965 | −0.3465 | 0.5745 | −0.2105 |
| ATP6V1G [symbol] | 0.285 | 0.381 | 0.082 | −0.753 | −1.214 | −0.379 | −0.438 | −0.356 | −0.078 | 0.487 |
| ATPIF1 | −0.0125 | 0.0235 | −1.4335 | −1.2285 | −1.1235 | −0.5935 | −0.8805 | −0.7505 | −0.7585 | −0.2785 |
| ATXN10 | −1.377 | −0.853 | −0.549 | −0.902 | 0.06 | −0.644 | 0.744 | −0.041 | −0.737 | 0.229 |
| B3GNT5 | 0.5235 | 0.0885 | 0.8865 | 0.1365 | −0.4245 | 0.4385 | 0.6655 | 0.5595 | 0.2835 | 1.8415 |
| BACE2 | 0.5485 | 0.1826 | 0.0395 | 0.1985 | 0.1015 | 0.2195 | −0.0775 | 0.0145 | 0.5995 | −0.0095 |
| BAG1 | 2.5825 | 1.1965 | 0.8455 | 0.9895 | 1.2695 | 1.7285 | 0.1055 | 0.5085 | 0.4755 | 1.1825 |
| BAG4 | −2.7845 | 2.3905 | 2.1045 | 4.2075 | −2.5725 | −2.5685 | 0.2875 | 0.6085 | 2.2855 | 2.2125 |
| BAI2 | −0.116 | 0.403 | −0.214 | 1.014 | 1.725 | 1.372 | 0.59 | 1.777 | −0.407 | 0.07 |
| BAMBI | −0.435 | −0.264 | 0.191 | 1.878 | −0.283 | 0.465 | 0.079 | 0.245 | −1.042 | −0.488 |
| BCAS1 | 1.557 | −2.658 | 1.336 | 1.794 | 2.188 | 3.194 | 1.793 | 2.675 | 0.71 | 1.076 |
| BCL2L14 | 0.5415 | 4.1515 | 2.0155 | 2.8245 | −1.1775 | −1.3785 | −0.4585 | −1.2685 | −0.7245 | 3.5155 |
| BCMP11 | 0.251 | 2.066 | 0.972 | 2.079 | −1.749 | −2.067 | −0.991 | −1.936 | −1.931 | 1.318 |
| BEX1 | 1.5615 | 2.2055 | 1.0815 | 2.3145 | 2.8295 | 2.0435 | 2.3805 | 2.1955 | 0.4415 | 1.1065 |
| BEX2 | 1.1565 | 0.1125 | 0.6685 | −1.7585 | −0.3475 | −0.5705 | 1.4665 | 2.2025 | 1.1185 | −0.1455 |
| BF | −0.084 | 0.951 | 0.573 | 0.868 | 0.067 | 1.104 | −0.026 | −0.164 | 0.132 | 0.066 |
| BFSP1 | −0.196 | −0.092 | 0.073 | −0.96 | −0.307 | −0.21 | −0.107 | −0.195 | 0.138 | −0.098 |
| BIK | −0.755 | −1.232 | −0.033 | −2.066 | −1.142 | −1.645 | −0.643 | −0.458 | −0.522 | −2.484 |
| BIRC2 | −0.4735 | 0.0985 | −0.0205 | −1.1945 | −0.2295 | −0.3525 | −0.0015 | 0.0015 | −0.0945 | −0.3665 |
| BIRC5 | −0.2705 | 0.8705 | 0.6405 | −0.5005 | −1.1175 | −0.8825 | −0.1205 | 0.9625 | −0.1815 | −0.0215 |
| Bit1 | 0.6765 | −0.6275 | −1.3785 | 0.4075 | 0.4265 | 1.4595 | 0.8585 | 1.4045 | −0.0085 | −0.4605 |
| BIVM | −0.287 | −0.807 | −0.281 | 0.077 | −0.658 | −1.07 | −0.192 | −0.739 | 1.153 | −1.183 |
| BLVRA | 0.769 | 0.199 | −0.419 | −0.771 | −0.195 | −0.691 | −0.231 | −1.223 | −0.09 | 0.081 |
| BM039 | 0.06 | −0.109 | 0.102 | −0.069 | 0.998 | −0.001 | 1.315 | 0.355 | −0.339 | 0.205 |
| BMP7 | 0.186 | −0.493 | 0.72 | −0.793 | −0.175 | −0.285 | 0.055 | 0.14 | −0.09 | −0.169 |
| BOC | −0.0915 | 0.1875 | −0.3545 | 0.3285 | −0.3425 | −0.2465 | 0.0865 | −0.2245 | 1.3895 | 1.0615 |
| BRCA1 | −0.0945 | −0.2955 | −0.7865 | −0.6545 | −0.4715 | −0.8135 | −0.4175 | −1.1225 | −0.4395 | −0.3265 |
| BRF2 | 1.314 | −0.413 | −0.073 | 0.488 | 1.309 | −0.383 | 0.207 | 0.206 | −0.251 | 0.391 |
| BRI3 | −0.151 | 0.899 | 0.452 | 0.746 | 0.256 | 0.303 | 0.541 | 0.429 | 0.832 | −1.085 |
| BRI3BP | 0.274 | 0.064 | 0.262 | 0.932 | −0.418 | 0.652 | 0.375 | 0.598 | −0.159 | −0.434 |
| BSPRY | −0.7715 | −1.1955 | 0.0815 | −2.9575 | −1.7855 | −1.5255 | −1.2435 | −2.3385 | −0.6305 | −0.2155 |
| BTF3 | −0.2315 | −0.4605 | −0.3265 | 0.0355 | −0.0875 | −0.2855 | −0.2305 | −0.2455 | 0.0265 | −0.1315 |
| BTG3 | −0.235 | −1.148 | −0.502 | −0.931 | −1.684 | −1.785 | −0.835 | −0.521 | −0.291 | −1.05 |
| BγN2A2 | 0.047 | 0.027 | 0.188 | 0.383 | −1.291 | 0.252 | −0.028 | 0.706 | 0.243 | −0.927 |
| BUB1 | −0.0015 | −0.0895 | −0.0875 | −0.2075 | 0.4255 | −0.1785 | 0.1385 | 0.0535 | −0.9685 | −0.7725 |
| BUB3 | 0.165 | −0.315 | 0.088 | −0.866 | −0.637 | −0.221 | −0.548 | −0.694 | −0.234 | −0.395 |
| BXDC1 | −0.484 | 0.804 | −0.745 | 0.698 | −1.681 | 0.004 | 0.028 | 0.199 | 0.367 | −0.216 |
| BZRP | 1.014 | 0.033 | 0.049 | 1.585 | 0.463 | 0.926 | 0.372 | 0.296 | 0.766 | 0.994 |
| BZW1 | −0.5585 | −0.5195 | −0.3345 | −0.2845 | 0.0215 | −0.1635 | 0.0855 | −0.7745 | −0.8635 | 0.4155 |
| C10orf116 | 0.7355 | −0.2475 | 0.6275 | −0.2755 | 0.0725 | −0.0685 | 0.5615 | 0.1815 | 0.1005 | 0.4205 |
| C10orf38 | −0.642 | 0.032 | 0.02 | 1.173 | −0.314 | 0.687 | 0.088 | 0.332 | −0.141 | −0.2 |
| C10orf4 | −0.2755 | −0.0635 | −0.1015 | −0.2565 | −0.2095 | −0.2055 | −0.0945 | −0.2365 | 0.0855 | −0.0065 |
| C10orf45 | −0.2265 | −1.2125 | 0.4715 | 2.9855 | 0.8535 | 0.8075 | −0.7895 | −0.8865 | 0.1235 | 0.5595 |
| C10orf7 | −0.4215 | −0.1435 | −0.0045 | −0.1605 | 0.4405 | 0.2235 | 0.0045 | −0.5015 | −0.2225 | 0.4095 |
| C10orf82 | −0.341 | 0.083 | 0.742 | −0.114 | 0.496 | −0.814 | 0.567 | −0.472 | −0.366 | −0.03 |
| C11orf24 | −1.1975 | 1.0425 | 0.8345 | −0.4355 | 0.6745 | 0.1495 | −0.2325 | −1.4535 | 1.4405 | 0.6785 |
| C12orf14 | −0.049 | 0.155 | 0.3 | −0.895 | −0.192 | −0.27 | −0.327 | −0.14 | 0.063 | −0.479 |
| C13orf21 | −0.377 | −0.227 | 0.604 | −0.38 | −0.034 | 0 | 0.273 | −0.026 | −0.119 | −0.267 |
| C13orf23 | −0.078 | −0.806 | −0.171 | −0.456 | −1.203 | −1 | −0.974 | −0.832 | −0.067 | −0.752 |
| C14orf94 | 1.257 | 0.59 | 0.55 | 0.77 | 1.08 | −0.12 | −0.36 | −0.93 | 2.1 | 1.54 |
| C15orf23 | 0.0945 | −0.7095 | 0.0505 | 0.9415 | 0.2055 | 0.2825 | −0.0615 | −0.1445 | −0.0705 | −0.2805 |
| C16orf45 | −0.571 | 0.315 | 0.523 | 1.874 | 0.536 | 0.043 | 0.302 | 0.424 | 1.219 | 1.134 |
| C17orf37 | 0.3 | −0.179 | −0.283 | −0.012 | −0.024 | 0.183 | 0.319 | 0.234 | −0.079 | 0.536 |
| C18orf1 | 1.652 | 1.884 | 0.804 | 1.623 | 1.671 | 1.261 | 1.013 | 1.248 | 1.216 | −0.295 |
| C19orf10 | −0.5825 | −1.4445 | −0.4525 | 0.3065 | 0.4645 | −0.5475 | −0.0745 | 0.8275 | 2.0585 | 0.5095 |
| C19orf32 | 1.1375 | 1.3445 | 1.3295 | 0.7345 | 0.8485 | 1.6005 | 1.0495 | 1.5835 | 0.0205 | 0.7225 |
| C19orf33 | 0.265 | 1.659 | 1.877 | 2.187 | −0.216 | 0.699 | 1.813 | 2.282 | 2.498 | −0.335 |
| C1orf24 | −1.8 | 1.598 | 0.919 | 0.728 | 1.404 | 0.978 | 1.772 | 0.681 | 1.161 | 0.322 |
| C1orf34 | 0.5245 | −0.3605 | −0.1135 | 0.8255 | 0.8985 | 0.5805 | 0.3985 | 0.3185 | 0.3395 | 0.0915 |
| C20orf103 | 0.6235 | 0.8585 | 0.7325 | −0.1315 | −0.5935 | 0.7225 | 0.8205 | 1.4585 | −0.0415 | −0.1815 |
| C20orf149 | 0.383 | 0.557 | −0.279 | 0.227 | 0.074 | 0.260 | −0.031 | 0.252 | −0.126 | −0.042 |
| C20orf23 | 0.263 | 0.45 | 0.994 | 1.668 | 0.61 | 0.975 | 0.807 | 1.311 | −0.203 | 0.257 |
| C20orf3 | −0.397 | 0.14 | −0.09 | 0.457 | −0.541 | −0.385 | −0.052 | 0.016 | 0.086 | −0.226 |
| C20orf35 | 0.669 | 0.134 | −0.534 | −0.441 | −0.133 | −0.692 | −0.277 | −0.357 | −1.104 | −0.35 |
| C20orf43 | 0.0195 | −0.3155 | −0.2375 | 0.1665 | −0.3225 | −0.5995 | 0.1845 | 0.0285 | −0.0035 | −0.1275 |
| C20orf8 | −0.213 | −0.588 | 0.638 | −1.072 | −0.369 | −0.182 | −0.634 | −0.839 | −0.852 | −0.91 |
| C21orf45 | 0.075 | −0.546 | −0.121 | −0.588 | −0.324 | −0.184 | 0.285 | −0.016 | −0.174 | 0.07 |
| C22orf18 | −0.097 | −0.493 | −0.005 | −0.225 | −0.758 | −0.139 | 0.57 | 0.282 | 0.23 | −0.307 |
| C2F | 0.464 | 0.796 | 1.483 | 0.615 | 1.827 | 1.923 | 1.568 | 0.361 | −0.567 | 1.34 |
| C2orf17 | −0.711 | −0.047 | 0.181 | −0.353 | 0.255 | −0.466 | −0.417 | −0.876 | 0.008 | 1.243 |
| C4A | −1.017 | −0.189 | 0.014 | −0.711 | −0.922 | −1.295 | 0.218 | 0.129 | −0.351 | −0.849 |
| C5orf13 | 0.304 | −0.288 | −0.645 | 0.273 | −0.367 | −0.432 | −0.059 | 0.301 | −0.261 | −0.273 |
| C6orf108 | −0.078 | −0.297 | −0.052 | −1.058 | 0.199 | −0.124 | 0.039 | 0.433 | −1.605 | −0.281 |
| C6orf166 | 1.152 | 1.506 | 0.264 | 0.241 | −1.009 | −0.225 | 0.458 | 0.739 | 2.631 | 2.245 |
| C6orf192 | −0.0855 | −0.0965 | −0.1305 | 0.4585 | −0.4675 | −0.0805 | 0.1345 | −0.1365 | −0.0555 | −0.2175 |
| C6orf211 | 0.237 | 0.449 | 1.214 | −0.049 | 0.761 | 0.271 | 1.413 | 1.596 | 1.103 | −0.222 |
| C6orf49 | −0.7055 | 0.8195 | 0.2065 | −0.8445 | −0.6635 | −0.1165 | 0.4755 | 0.8805 | −0.4495 | −0.6585 |
| C6orf85 | 0.7985 | 0.9965 | 0.7816 | 0.8795 | 1.0045 | 0.9145 | 0.8885 | 1.0055 | −0.0765 | 0.6795 |
| C7orf24 | 1.513 | 1.047 | 1.153 | 1.834 | 1.451 | 2.346 | 3.182 | 3.286 | 3.881 | −0.2 |
| C9orf10 | 0.165 | −2.355 | −1.564 | −1.963 | −2.244 | −1.23 | 0.056 | −0.617 | −1.755 | −0.482 |
| C9orf121 | −0.069 | −0.696 | −0.072 | −0.015 | 0.664 | 0.144 | 0.125 | 0.072 | −0.08 | 0.181 |
| C9orf58 | 0.3545 | 0.1435 | 0.3215 | 0.1305 | −0.7435 | 0.1445 | −0.0405 | −0.2975 | 0.3205 | 0.0625 |
| C9orf88 | 2.151 | 1.452 | 0.641 | 1.433 | 1.903 | 2.061 | 1.311 | 1.4 | 0.755 | 0.628 |
| C9orf9 | 1.124 | 1.065 | 0.952 | 1.829 | −0.043 | 1.354 | 0.63 | −0.442 | 0.836 | −0.541 |
| CA12 | 0.891 | 1.107 | 0.853 | 1.867 | −0.255 | 1.366 | 0.635 | −0.539 | 1.06 | −0.192 |
| CA8 | 0.589 | 0.312 | −0.275 | 0.687 | −0.379 | 0.275 | 0.162 | −0.241 | −0.029 | 0.42 |
| CA8 | 0.2925 | 0.0105 | −0.1965 | −0.4655 | −0.1035 | −0.0775 | −0.1025 | 0.2095 | 0.0985 | −0.2095 |
| CACNA1G | 1.067 | −0.087 | −0.087 | −1.321 | −0.027 | −0.674 | −0.184 | −1.281 | −0.627 | 0.155 |
| CALCA | −0.313 | 1.404 | −0.568 | −0.099 | 2.181 | −0.695 | −0.515 | −0.956 | −0.445 | −0.848 |
| CALML4 | −0.2415 | −1.0065 | 0.1335 | 0.8515 | −0.6415 | −0.6105 | −0.7095 | −1.3185 | 0.7205 | 0.2105 |
| CALML5 | 1.1115 | 0.5685 | 0.5225 | 2.2755 | 0.6785 | 0.8155 | 2.1365 | 2.9405 | 1.3995 | −0.4425 |
| CaMKIINa [symbol] | 0.498 | 0.367 | 0.523 | −0.64 | −0.31 | −0.245 | −0.143 | 0.369 | −0.053 | −0.516 |
| CAP2 | 0.98 | 1.064 | 1.062 | 1.893 | 1.52 | 1.386 | 1.18 | 1.551 | 0.768 | −0.437 |
| CAP350 | 0.1575 | −1.9605 | −0.3805 | −1.4185 | −1.7915 | −1.8795 | 1.1235 | −1.0765 | −0.7285 | 0.0375 |
| CAPN13 | −0.976 | 1.324 | 0.357 | −0.017 | 2.27 | 1.076 | 0.939 | 1.368 | 0.2 | −0.172 |
| CAPN9 | 0.1855 | 0.2505 | 0.1425 | −0.0475 | −0.0325 | 0.8235 | 0.5885 | 1.8275 | 0.8795 | −0.0015 |
| CARD14 | −0.2495 | 0.2355 | −1.0015 | −1.3225 | −0.7925 | 0.0805 | −0.4355 | −0.6485 | −1.1045 | −0.4485 |
| CART | −0.1325 | −0.2985 | −0.0375 | 0.1725 | 0.0575 | −0.1135 | −0.0085 | 0.3925 | 0.0255 | −0.1885 |
| CASP1 | 0.365 | 0.054 | −0.197 | 0.291 | −0.126 | 0.781 | 0.184 | 0.123 | 1.315 | 0.999 |
| CASP3 | −0.557 | −0.143 | 0.417 | −0.108 | 0.729 | 0.758 | 0.119 | −0.118 | −0.237 | −0.355 |
| CASP7 | 0.6835 | −1.2155 | −0.0155 | −1.2955 | −0.4555 | −1.9155 | 0.3945 | −0.2955 | −0.7855 | 0.4045 |
| C9LN1 | 0.113 | 0.665 | 1.144 | −1.459 | 1.062 | −0.503 | 0.622 | 0.352 | −0.619 | −0.508 |
| CBR1 | 0.7775 | 0.8105 | 0.8385 | 0.2705 | −0.2045 | 0.7015 | 0.5235 | 0.5385 | −0.2015 | −0.3645 |
| CBR3 | 0.233 | −0.172 | 0.181 | 0.284 | 0.465 | 0.176 | 0.309 | −0.217 | −0.336 | 0.025 |
| CBR4 | 1.0485 | −1.7505 | −1.4465 | −0.3275 | 2.2735 | 0.0535 | 1.8765 | 3.3855 | −0.2255 | −0.7985 |
| CCL24 | −0.683 | −0.539 | −0.191 | −0.992 | −0.956 | −1.441 | −0.877 | −1.279 | −0.218 | −1.676 |
| CCL29 | 0.601 | 0.172 | −0.184 | 0.078 | 0.267 | 1.749 | −0.839 | −1.003 | 1.098 | 1.131 |
| CCNB2 | 0.0625 | −0.4965 | −0.5295 | −0.3935 | −0.7625 | −0.5715 | −0.5535 | −0.9965 | −0.4675 | −0.3375 |
| CCND1 | −0.444 | 0.873 | 1.729 | 1.549 | −0.165 | 1.04 | 1.384 | 1.877 | 0.57 | −0.473 |
| CCNE1 | −0.0405 | 0.1985 | −0.0065 | 0.1075 | −0.3825 | 0.2885 | 0.0925 | 0.2415 | 0.1295 | 0.4595 |
| CCNG2 | −1.106 | −1.085 | −0.955 | −0.107 | −0.923 | −1.135 | −0.893 | −0.879 | −0.559 | −0.815 |
| CCNH | −0.1975 | −0.9815 | −0.6965 | 0.0255 | −0.3045 | −0.7205 | −0.1705 | −0.1395 | −0.8905 | −0.6285 |
| CD38 | 0.0115 | −0.2765 | −0.1375 | 0.0945 | 0.3145 | −0.1576 | −0.2155 | −0.1505 | −0.2375 | −0.1095 |
| C030 | −0.0815 | 0.4875 | −0.1475 | −0.8595 | −0.7505 | −0.1275 | −1.1525 | −0.4375 | −0.4545 | −0.0615 |
| CD4 | 0.2665 | 0.2425 | −0.4085 | −0.8695 | −0.5745 | −0.0595 | 0.0105 | −0.3895 | 0.0315 | 0.1045 |
| CD58 | −0.214 | −0.686 | 0.044 | −1.246 | −1.455 | −1.512 | −1.314 | −0.497 | −0.513 | −1.286 |
| CD50 | 0.0075 | −0.9975 | −0.2055 | −0.9745 | −1.2265 | −1.3935 | −1.2825 | −0.9735 | −0.8835 | −0.7465 |
| CDC2 | −0.8985 | 1.3255 | 0.0095 | −2.3055 | −1.4315 | −1.7585 | −1.1885 | −1.0325 | −0.8095 | −0.9265 |
| CDC6 | −1.1845 | −1.5725 | −0.0625 | −1.4985 | −1.4555 | −1.9435 | −0.5995 | −0.7355 | −0.1835 | −1.7695 |
| CDCA1 | 0.048 | −1.542 | −0.668 | −1.55 | −1.403 | −1.265 | −0.021 | −0.473 | −1.717 | −0.787 |
| CDCA5 | −0.314 | 0.803 | −0.46 | 0.8 | −0.3 | 0.422 | −1.909 | −3.734 | 0.95 | −0.577 |
| CDCA7 | −1.3845 | −0.3155 | −0.1955 | −1.4895 | −0.0475 | −0.6955 | −0.3445 | −2.0385 | −0.6585 | −0.8735 |
| CDH1 | 0.292 | −0.288 | 0.178 | 0.86 | 0.16 | −0.217 | 0.294 | −0.222 | 0.549 | 0.828 |
| CDH3 | −0.1835 | −0.0365 | 0.0905 | −0.0495 | −0.1255 | −0.3515 | −0.1355 | −0.8905 | −0.2445 | −0.5445 |
| CDK2 | −0.6075 | −1.0165 | −0.3805 | −0.3635 | −1.1245 | −1.0785 | −0.9795 | −0.7475 | 1.0345 | 0.0165 |
| CDKN2A | −0.4305 | −0.0505 | −0.2875 | −0.6795 | −0.7995 | −1.2045 | −0.5045 | −0.1765 | −0.2475 | −0.9105 |
| CDKN2D | 0.222 | −0.887 | −0.267 | −0.495 | −0.854 | −0.926 | −0.401 | −1.037 | −0.547 | −0.453 |
| CDKN3 | −0.246 | −0.449 | −0.733 | −0.699 | −0.366 | −0.306 | −0.318 | −0.118 | 0.251 | −0.164 |
| CDT1 | 1.0495 | 0.9365 | 1.9105 | 0.0095 | 1.4055 | 2.4995 | 1.4165 | 1.5215 | 0.7395 | 0.5235 |
| CE9PG | 0.054 | −1.301 | −0.586 | −0.983 | −1.091 | −1.444 | −0.956 | −0.785 | −0.194 | −0.973 |
| CELSR1 | −0.366 | −0.931 | −0.211 | −0.469 | −1.354 | −1.562 | −0.648 | −0.538 | −0.282 | −0.762 |
| CENPA | −0.454 | −1.024 | 0.303 | −2.329 | −0.697 | −1.649 | −0.712 | −0.159 | −0.469 | −2.116 |
| CENPE | −0.482 | −0.511 | −0.076 | −0.697 | 0.63 | −0.258 | −0.606 | −1.55 | 0.27 | 0.299 |
| CENPF | −0.0225 | 0.0745 | −0.1436 | 0.4895 | 0.0935 | −0.0495 | 0.2885 | 0.0625 | −0.6915 | −0.3735 |
| CENTG3 | 0.2345 | 0.2755 | −0.2115 | 0.8595 | −1.7795 | −0.5385 | −0.1935 | −0.0725 | 0.1415 | 0.1375 |
| CES2 | 0.113 | −0.762 | −0.352 | −0.473 | −0.821 | −0.407 | −0.591 | −1.041 | −0.997 | 0.2 |
| CETN3 | −0.8675 | −0.0265 | −0.4715 | −1.5305 | −1.0325 | −1.1605 | −0.3885 | −0.7195 | 1.1235 | 0.0035 |
| CGA | −0.1685 | 0.0425 | 0.2575 | −1.0675 | −1.1625 | −0.8195 | −0.1095 | −0.3325 | −0.1205 | 0.2095 |
| CGI-12 | 0.4975 | 1.7505 | 0.2805 | 0.9725 | 0.2285 | 0.5815 | 0.4455 | 0.8675 | 0.4045 | −0.4665 |
| CGI-49 | −0.11 | 1.281 | 0.446 | 1.203 | 0.828 | 0.327 | 0.888 | 0.477 | 0.883 | 0.257 |
| CGI-4 [symbol] | −0.06 | 0.476 | 1.882 | −0.071 | 0.331 | 1.856 | 1.427 | 1.395 | 0.538 | 0.777 |
| CGN | −0.197 | −1.011 | −0.074 | −0.938 | −0.597 | −0.793 | −0.246 | −0.02 | 0.279 | −0.954 |
| CGNL1 | −0.4035 | −1.0175 | 0.1405 | −0.6795 | 0.0925 | −0.4275 | −0.6125 | −1.2755 | 1.1185 | −1.4125 |
| CHAF1B | −0.052 | −0.467 | −0.146 | −0.057 | −0.187 | −0.081 | 0.49 | 0.745 | 0.182 | 0.123 |
| CHC1 | 0.857 | 0.292 | 0.471 | 1.027 | 0.005 | 0.748 | 1.015 | 1.085 | 0.628 | 0.442 |
| CHCHD2 | −0.759 | 1.333 | −0.978 | −1.628 | −1.451 | −1.095 | −0.154 | −1.853 | −0.507 | −0.929 |
| CHCH05 | 0.513 | 3.449 | 1.339 | −1.529 | 1.076 | −0.14 | −0.011 | 0.479 | −1.349 | −0.598 |
| CHI3L1 | −0.658 | −0.098 | 0.119 | 0.078 | 0.429 | 0.268 | 0.042 | −0.083 | 0.095 | −0.572 |
| CHI3L2 | −0.294 | −0.454 | −0.138 | 0.032 | −0.077 | −0.179 | −0.248 | −0.311 | −0.06 | −0.055 |
| CHKA | 0.093 | 1.394 | 0.287 | −0.255 | 0.01 | −0.052 | −0.025 | −0.28 | −0.003 | −0.002 |
| CHCDL | −0.1165 | −0.2185 | −0.0805 | 0.2916 | 0.3365 | −0.0905 | −0.1035 | −0.0825 | 0.0135 | 0.6945 |
| CHRDL2 | 0.805 | 0.831 | 0.032 | 1.085 | −0.104 | 0.236 | 0.073 | 0.252 | −0.016 | 0.017 |
| CHST2 | −0.3815 | 0.2525 | −0.0275 | −0.0865 | 0.0065 | 0.1075 | 0.1815 | −0.0025 | −0.0475 | −0.1855 |
| CHURC1 | 0.195 | 0.366 | 0.387 | −0.165 | 0.112 | 1.024 | 0.393 | 0.173 | 0.52 | 0.481 |
| CIR | −0.1665 | 0.4925 | −0.1845 | 0.3535 | −0.3735 | −0.3245 | −0.3805 | −0.3225 | −0.4305 | −0.3205 |
| CIRBP | 0.1455 | −1.3335 | −0.5515 | 0.6535 | −0.9775 | 0.0435 | −0.4325 | −0.1185 | 0.3365 | 0.5065 |
| CKLF | −0.2905 | −1.2295 | 1.3635 | 1.3115 | −0.3005 | −0.4195 | −0.3145 | −0.9835 | 1.8785 | 1.0975 |
| CKLFSF6 | −0.1065 | −0.5015 | −0.9165 | −1.9375 | 0.3445 | −1.5665 | −0.2875 | −0.8685 | −0.0115 | −1.3375 |
| CKLFSF7 | −0.004 | −0.04 | 0.269 | −0.074 | −1.272 | −1.251 | −0.288 | −0.091 | −0.493 | −1.335 |
| CKMT1 | 0.326 | −1.344 | −0.131 | 0.725 | −1.717 | −1.503 | −0.633 | 0.024 | −0.018 | −0.85 |
| CKS1B | −0.665 | −0.422 | 0.224 | −0.788 | −0.601 | −0.478 | −0.527 | −0.495 | −0.248 | −0.34 |
| CKS2 | −0.8975 | −0.0005 | −0.0825 | −0.3435 | −1.6275 | −1.1215 | −1.0045 | −0.7225 | 1.3705 | −0.7005 |
| CL640 | 0.0025 | 0.6565 | 0.3825 | 0.3445 | −0.0175 | −0.9055 | −0.0205 | −0.2115 | 1.7755 | 0.4875 |
| CLDN23 | −1.4885 | 0.0255 | −0.1035 | 0.9305 | −0.0515 | −0.4485 | 0.4235 | 0.3145 | 0.2775 | −0.0705 |
| CLDN3 | −0.8735 | 0.5755 | −0.6005 | −1.1085 | −0.7995 | −0.9965 | 2.0225 | 1.3765 | 0.9955 | −0.9555 |
| CLDN4 | 1.939 | 2.158 | 2.312 | 1.276 | 0.257 | 0.868 | 0.411 | 0.344 | −2.509 | −0.858 |
| CLDN8 | −0.2835 | 2.6035 | 3.5525 | 0.8545 | −1.0155 | 0.9405 | 2.1815 | 0.2735 | 3.1025 | 2.8155 |
| CLGN | −0.2055 | −0.0795 | 0.1415 | −0.2645 | −0.4575 | −0.0445 | 0.2595 | 0.0175 | 0.0705 | −0.5295 |
| CLIC6 | 0.177 | 0.29 | −0.396 | −0.797 | −0.796 | −0.416 | −0.064 | 0.102 | 0.229 | 0.167 |
| CLNS1A | 0.5155 | 1.8195 | −1.1335 | −0.0035 | −1.0685 | −1.4785 | −0.0555 | −0.1405 | −0.9435 | 0.3116 |
| CLTC | −0.225 | −0.017 | −0.238 | −0.095 | −0.792 | −0.694 | 0.418 | 0.346 | 0.081 | 0.13 |
| CLU | −0.38 | −0.328 | −0.72 | −0.402 | −1.572 | −1.267 | −0.768 | −0.543 | 0.441 | −0.327 |
| CMAS | 0.1295 | 1.9505 | 0.1715 | −0.0775 | −1.0595 | −0.4655 | −0.7795 | 0.3715 | −0.8345 | −1.1475 |
| CML65 | −0.025 | −0.244 | 0.040 | 0.347 | 0.392 | 0.019 | 0.207 | 0.343 | 0.201 | 0.062 |
| CNIH | −0.408 | −0.101 | 0.041 | 0.305 | 0.48 | −0.018 | 0.168 | 0.001 | 0.428 | 0.434 |
| CNOT10 | −0.1515 | −0.0005 | 0.2545 | −0.0505 | −0.2555 | 0.0255 | 0.2395 | 0.4375 | −0.3255 | −0.6305 |
| CNOT2 | −0.318 | −0.262 | −0.229 | 0.349 | −0.006 | 0.023 | −0.084 | −0.227 | −0.595 | −0.648 |
| CNOT7 | 0.288 | 0.075 | 1.919 | −1.463 | −0.04 | −1.26 | 2.636 | 2.194 | 3.705 | 1.478 |
| CNP | 0.2095 | −0.1005 | 0.4025 | 0.2735 | 0.5315 | 0.3515 | 0.7145 | 0.5865 | 0.8815 | 0.3495 |
| CNTNAP2 | −0.436 | −0.564 | −0.114 | 0.213 | −0.072 | −0.016 | −0.21 | −0.53 | −0.562 | −0.234 |
| COASY | −0.4975 | 0.6025 | 0.4835 | −1.6205 | 1.3015 | −0.2195 | 0.9345 | 0.5185 | −0.7065 | 0.1725 |
| COL11A2 | 1.181 | 1.622 | 1.627 | 0.248 | −0.068 | 0.625 | 0.355 | 0.386 | −0.631 | −0.047 |
| COL27A1 | −0.447 | −0.894 | −0.156 | −0.324 | 0.028 | −0.023 | −0.038 | −0.48 | −0.291 | −0.795 |
| COL4A5 | −0.3395 | −0.5215 | 0.7705 | −1.9225 | −0.7995 | −0.7205 | −0.6455 | −1.3595 | 0.0975 | −0.5975 |
| COL8A1 | −0.179 | 0.921 | −0.006 | 0.216 | −1.239 | 0.541 | 0.484 | 0.682 | 0.256 | 0.05 |
| COL8A3 | −0.206 | −0.123 | −0.366 | −0.271 | −0.272 | −0.296 | −0.029 | 0.158 | 1.106 | 0.13 |
| COMMD1 [symbol] | −0.0455 | −0.3855 | 0.0215 | −0.2525 | −0.8025 | −0.2245 | 0.0405 | −0.1115 | −0.0205 | −0.0665 |
| COMMD5 | 0.7815 | −0.3015 | −0.3635 | 0.4415 | 2.0835 | −0.0335 | 2.1485 | 3.2085 | 0.4495 | −0.1335 |
| COPS7B | 0.098 | −0.192 | −0.116 | 0.412 | −0.144 | −0.24 | −0.44 | −0.779 | −0.045 | −0.583 |
| CORO6 | 0.1175 | −0.0675 | −0.1525 | 1.4765 | 0.1195 | 0.0405 | −0.1445 | 0.0895 | −0.2105 | −0.0625 |
| CORT | −0.164 | −0.202 | −0.164 | 0.176 | −0.879 | −0.279 | −0.595 | −0.397 | −0.174 | −0.038 |
| COX17 | −0.298 | −0.105 | 0.037 | 0.22 | −0.851 | −0.229 | −0.576 | −0.498 | −0.334 | −0.445 |
| COX5A | 0.228 | 0.068 | −0.07 | 0.7 | −0.099 | 0.132 | 0.55 | 0.851 | 0.086 | −0.01 |
| COX6A | 1.8305 | −0.6355 | 0.6215 | 2.7655 | 1.3025 | 1.7945 | −0.0215 | 0.8035 | 1.0745 | 1.1655 |
| COX8A1 | 0.5335 | 0.0975 | −0.4295 | 0.7195 | −0.1285 | 0.1715 | 0.1045 | 0.3995 | −0.2275 | −0.1115 |
| COX8C | −0.0075 | −0.7905 | −1.3955 | −0.9175 | −1.4585 | −0.9965 | 2.0525 | −0.5465 | −1.3035 | −0.3915 |
| COX7A2 | −0.285 | −0.175 | −0.403 | −0.679 | 0.012 | −1.009 | −0.133 | −0.817 | 2.447 | 0.196 |
| CP | −1.0635 | −0.4415 | 0.5225 | −1.3605 | 1.6545 | 3.1815 | 0.2375 | 2.4065 | 0.0825 | 0.4955 |
| CPAMD0 | 0.067 | 1.214 | 0.662 | 0.238 | 0.914 | 1.205 | 0.838 | 1.06 | 0.874 | 0.219 |
| CPB1 | 0.4705 | 0.9975 | 0.6545 | 0.6385 | 0.1255 | 0.3275 | −0.0265 | 0.8805 | −0.0815 | 0.4105 |
| CPEB3 | 0.161 | −0.28 | 0.047 | −0.265 | 0.035 | −0.212 | 0.099 | 0.128 | −0.118 | 0.14 |
| CPEB4 | 0.045 | 1.422 | 0.43 | −0.081 | 0.408 | 0.001 | 0.556 | −0.828 | −0.885 | −0.881 |
| CPSF3 | 1.4185 | 1.3255 | 1.1565 | 0.4835 | 1.6965 | 0.6205 | 0.8305 | 1.3665 | 0.7525 | −1.6665 |
| CRABP1 | 0.364 | 0.812 | 0.565 | 1.202 | 0.76 | 0.814 | 0.082 | 0.484 | 0.796 | 0.355 |
| CREB3L4 | −0.144 | −0.511 | −0.146 | 0.072 | −0.166 | −0.19 | 1.078 | 0.834 | −0.306 | −0.408 |
| CRIP1 | 0.211 | −0.398 | 0.071 | 0.295 | −0.559 | 0.13 | 0.684 | 1.076 | −0.053 | 0.118 |
| CRYBB2 | 0.008 | 0.955 | 0.29 | 0.31 | −1.599 | −0.68 | 0.86 | 1.358 | 1.448 | 0.088 |
| CRYL1 | −0.603 | 0.065 | 0.378 | −0.61 | −0.547 | −0.923 | −0.472 | −1.412 | 0.064 | 0.069 |
| CRYZ | −0.0315 | −0.4705 | 0.0385 | 0.8395 | −0.7925 | −0.5805 | −0.3085 | −0.2265 | −0.3305 | −0.5705 |
| CSDA | 0.1445 | −0.2755 | −0.0865 | 0.1735 | 0.2145 | −0.1165 | −0.1795 | 0.0295 | −0.0485 | 0.1815 |
| CSE1L | −0.2225 | 0.3145 | −0.0015 | −0.1965 | −0.7875 | −0.7345 | −0.2625 | −0.5805 | 0.0015 | −0.3485 |
| CSK | −1.0125 | −0.7405 | −0.6515 | −1.4475 | −0.4095 | −0.4325 | −0.1375 | −1.6425 | −0.8635 | 2.3475 |
| CSRP1 | 2.7335 | 1.6465 | 1.0065 | −1.0585 | −0.3325 | 0.0515 | 1.3115 | 2.0625 | 1.4165 | 1.1025 |
| CSRP2 | 0.539 | 0.9 | −0.596 | −0.158 | −0.256 | −0.744 | −0.463 | −0.359 | −0.088 | −0.482 |
| CST5 | 0.2795 | 1.2385 | −1.2285 | −0.6455 | −1.0245 | −1.1215 | 0.1345 | −1.4735 | 0.2165 | 1.0835 |
| CSTB | 0.394 | 0.003 | 1.047 | −0.305 | 0.899 | −0.022 | 0.458 | 0.188 | 0.831 | 0.127 |
| CTAG1B | −0.2805 | 0.3145 | 0.3885 | −0.2115 | 0.0885 | 0.2415 | 0.0655 | −0.0045 | 0.2835 | −0.0265 |
| CTBP2 | −0.45 | 0.233 | −0.312 | −1.627 | −1.218 | −0.289 | 0.36 | −0.235 | −0.201 | 0.257 |
| CTNNA1 | −0.237 | −1.604 | 0.606 | 1.534 | −1.003 | −2.609 | −1.171 | −1.859 | 0.659 | 1.874 |
| CTNNAL1 | −0.6525 | −0.2835 | 0.1515 | −1.5225 | −0.6005 | −0.8255 | −1.0695 | −1.2525 | −0.2405 | −0.5785 |
| CTNND2 | −0.646 | −1.811 | −1.068 | −1.465 | −2.019 | −1.786 | −0.577 | −0.675 | −0.896 | −0.893 |
| CTPS | −0.411 | −0.434 | −0.416 | −0.704 | −0.509 | −0.377 | −0.569 | 0.652 | −0.917 | 0.02 |
| CTSL2 | 0.0535 | −0.3885 | −0.3465 | 0.5515 | −0.0915 | −0.5325 | 0.0845 | −0.0735 | 0.1885 | −0.2485 |
| CTSS | −1.642 | −1.37 | −1.75 | −2.54 | −0.72 | −0.72 | −0.19 | −2.62 | −0.27 | −0.87 |
| CTTN | −2.7825 | 0.4005 | −0.4435 | 0.6975 | 0.9325 | −0.0015 | −1.5855 | 0.6075 | −0.8795 | −0.4705 |
| CX3CL1 | 0.3235 | 0.6405 | 0.1115 | 0.9515 | −0.3785 | −0.0155 | 0.5445 | 1.2685 | 0.0395 | −0.8415 |
| CXCL9 | −1.2225 | 0.0215 | 0.7755 | 2.0845 | −0.3835 | −1.1305 | 1.3555 | 1.1355 | −1.3725 | 0.3615 |
| CXorf39 | 0.581 | 1.576 | 0.764 | 0.892 | −0.434 | 1.455 | 0.867 | 1.247 | 0.41 | 0.366 |
| CXXC4 | 0.334 | 0.153 | 0.558 | 0.431 | 0.876 | 0.603 | 0.25 | 1.034 | −0.099 | −0.523 |
| CYB5 | 0.2275 | −0.3125 | −0.4515 | −0.2185 | −0.7445 | −0.3976 | −0.4085 | −0.1915 | 0.8465 | −0.2915 |
| CYB561 | 0.371 | −0.15 | −0.192 | 0.127 | 0.755 | 0.559 | 0.765 | 0.352 | 1.571 | 1.148 |
| CYC1 | 0.086 | 0.924 | 0.118 | 0.141 | 0.198 | −0.199 | −0.120 | 0.072 | −0.152 | −0.306 |
| CYHR1 | 3.4715 | 4.9825 | 2.3075 | 2.8795 | 3.9575 | 2.5525 | 3.4195 | 3.9995 | 1.5935 | −0.6855 |
| CYP2A13 | −0.565 | −0.467 | −0.065 | −1.174 | −0.385 | −0.287 | −0.557 | −1.321 | −0.651 | −0.138 |
| CYP4X1 | −0.1565 | 3.7825 | −0.0295 | 2.2105 | 0.9135 | 2.4115 | −0.0105 | 0.8845 | −1.8835 | −1.2035 |
| D15Wsu7 [symbol] | 0.3655 | 2.7215 | −0.3375 | 1.3715 | 0.6145 | 1.9965 | −0.4405 | 0.6605 | −1.4295 | −0.6285 |
| DACH1 | 0.327 | 4.109 | 0.391 | 2.446 | 0.252 | 1.993 | −0.208 | 0.854 | −2.278 | −1.543 |
| DACH1 | 0.491 | 0.285 | 0.61 | −0.409 | 0.138 | −0.346 | 0.222 | 0.348 | −0.004 | −0.729 |
| DACH1 | −0.269 | −0.38 | 0.054 | −0.853 | −0.143 | 0.398 | 0.032 | 1.081 | 0.384 | −0.331 |
| DAP3 | −0.5365 | 0.0445 | −0.1755 | 0.4405 | −0.8895 | 0.0945 | −0.2305 | −0.0495 | 0.2885 | 0.2585 |
| DAPP1 | −0.222 | 1.232 | −0.002 | 1.761 | −0.722 | −0.748 | 0.858 | 0.648 | −0.864 | −1.183 |
| DAZAP2 | −0.341 | −0.165 | −0.593 | −0.879 | −0.851 | −0.489 | −0.046 | −0.793 | 0.025 | 0.652 |
| DBI | −1.0495 | −0.3825 | −0.5835 | 0.4875 | −1.0515 | −1.0215 | −0.5385 | −0.9255 | −1.0165 | −0.8515 |
| D9N1 | 0.263 | −0.341 | 0.351 | −0.057 | 0.051 | 0.567 | 0.057 | 0.051 | −0.15 | 0.095 |
| DC13 | 0.2565 | 0.0995 | −0.2355 | −0.4905 | −0.0975 | −0.1665 | −0.3775 | −0.7185 | 0.7125 | 0.6015 |
| DCPS | 0.3285 | 0.0605 | −0.0035 | −0.1645 | −0.3605 | 0.4275 | −0.2035 | 0.1665 | −0.5095 | 1.1845 |
| DDHD2 | −0.4465 | −0.7825 | −0.2685 | −0.6675 | 0.2285 | −0.6655 | −0.1415 | −0.0035 | −0.0615 | −0.5105 |
| D0X17 | −0.4805 | −0.2855 | −0.1425 | −0.3935 | −0.8195 | −0.3105 | 0.0055 | 0.2035 | 1.2165 | −0.2885 |
| DDX39 | 0.272 | −0.981 | −0.517 | 0.053 | −0.573 | −1.05 | 0.228 | 0.374 | 0.113 | −0.467 |
| DDX48 | −0.6155 | −0.3605 | −0.6315 | 0.2415 | 1.0205 | 0.6785 | 0.3095 | 0.0235 | −0.3685 | −0.8025 |
| DDX58 | −0.5005 | 0.1075 | −0.2955 | −1.2355 | −1.4015 | −0.4705 | −0.4035 | −0.3575 | −0.5265 | −0.9465 |
| DEF6 | −0.061 | 0.5 | 0.22 | −1.435 | −0.257 | −1.071 | −0.697 | −0.734 | −0.273 | −0.035 |
| DEK | −0.465 | −0.262 | 0.166 | 0.147 | −0.898 | 0.231 | 0.007 | 0.462 | 0.28 | −0.405 |
| DHCR7 | −0.345 | 0.236 | −0.691 | −1.11 | −0.341 | −0.787 | 0.042 | −0.678 | 0.334 | 0.401 |
| DHRS7 | 0.2565 | 0.0245 | 1.3295 | −0.0015 | 0.7755 | 0.5625 | 0.5705 | 0.1995 | −0.3325 | 0.4815 |
| DHX57 | −0.5175 | 2.7895 | 0.3645 | 1.4695 | 1.0055 | 1.1515 | 1.1305 | −0.6225 | 0.4575 | −0.9865 |
| DIRC1 | 0.081 | 0.416 | 0.105 | 0.199 | 0.809 | −0.169 | 0.299 | −0.203 | −0.008 | −0.203 |
| dJ222E13 | 0.065 | 1.066 | 0.56 | 0.902 | 0.328 | 0.547 | 0.216 | −0.194 | 0.452 | −0.396 |
| DKFZP434 | −0.409 | −0.959 | −0.732 | −1.551 | −0.188 | −0.856 | −0.696 | −1.688 | 0.371 | 0.371 |
| DKFZp434 | −0.6525 | −1.0585 | −0.1435 | −0.6695 | −0.5455 | −0.8625 | −0.3825 | −0.8745 | −0.4565 | −0.3585 |
| DKFZP434 | 0.2 | −0.182 | −0.017 | 0.987 | 0.053 | 0.408 | 0.123 | 0.505 | 0.355 | 0.461 |
| DKFZp434 | 0.3465 | −0.3035 | 0.1525 | 1.3945 | 0.1895 | 0.1165 | 0.2905 | 0.0945 | 0.2425 | 0.4705 |
| DKFZP564 | 0.2495 | 0.2965 | 0.4755 | 0.2416 | −0.0735 | −0.1825 | 0.8335 | 0.6645 | 1.4895 | 0.5305 |
| DKFZp564 | −1.052 | 0.001 | −0.347 | −1.084 | −1.204 | −1.058 | −0.819 | −0.611 | 0.79 | 0.053 |
| DKFZP564 | 0.0145 | −0.0825 | −0.0985 | −0.2285 | −0.5925 | −0.1775 | −0.1105 | 0.3645 | 0.1765 | −0.0315 |
| DKFZP564 | 0.307 | 0.009 | 0.073 | 0.935 | −0.672 | −0.135 | −0.438 | −0.183 | −0.217 | −0.557 |
| DKFZP564 | 1.114 | 0.083 | 1.907 | 0.139 | 1.227 | 0.66 | 0.688 | 1.09 | 0.667 | −0.563 |
| DKFZp586 | 1.7685 | −0.7715 | −0.2085 | −0.3625 | −0.0995 | −0.8195 | 0.5315 | −0.8185 | −0.3035 | 0.7085 |
| DKFZp761 | 0.19 | −0.155 | 0.068 | 0.293 | 0.165 | −0.001 | 0.066 | −0.028 | 0.019 | −0.407 |
| DLK1 | 0.264 | 0.468 | −0.031 | −1.209 | 0.034 | −0.127 | −1.235 | −2.649 | 1.173 | 1.379 |
| DLX5 | 0.7585 | −0.3135 | 0.6685 | 0.4725 | 1.3535 | 1.3125 | 1.9645 | 2.3355 | −0.5045 | −0.1935 |
| DNAJA4 | 2.5945 | −0.9475 | 2.2185 | 0.7815 | 1.7075 | 2.2655 | 0.4255 | 1.6955 | −0.4135 | 1.6075 |
| DNAJC1 | 0.169 | 0.119 | 0.084 | 0.076 | −0.09 | −0.784 | 0.155 | 0.228 | −0.026 | −0.521 |
| DNAJC12 | 1.396 | 1.85 | 1.622 | 2.164 | 0.783 | 0.554 | 0.921 | 1.39 | 1.385 | 0.326 |
| DNAJD1 | −0.082 | −0.19 | 0.456 | −0.473 | 0.299 | −0.057 | −0.174 | 0.492 | 0.374 | −0.249 |
| DNALI1 | −0.358 | −0.392 | −0.482 | −0.167 | −0.872 | −1.338 | 0.07 | −0.135 | 0.438 | 0.525 |
| DNMT1 | −0.9625 | −0.1825 | −0.3645 | −1.8145 | −1.9855 | −1.6745 | 1.1975 | −0.2205 | −0.9755 | 0.6535 |
| DOX1 | 0.4585 | −0.3605 | 0.4325 | 0.5455 | −0.4665 | −0.2285 | −0.0245 | 0.0955 | −0.1475 | −0.3815 |
| DSCR1 | 0.0235 | 0.5335 | 0.3765 | 0.6515 | −0.1015 | −0.1885 | 1.0935 | 1.1275 | 0.7075 | −0.0735 |
| DSCR1L2 | −0.2095 | −0.6535 | −0.0215 | 0.0065 | −1.0335 | −0.8835 | −0.1925 | −0.0565 | 0.1675 | −0.3405 |
| DSP | 0.4115 | 0.4165 | 0.6925 | −0.2385 | 0.1495 | −0.2315 | 0.5155 | 0.3575 | −0.4725 | −0.4735 |
| DTYMK | 0.4375 | 1.4195 | 0.1455 | 1.8945 | 1.7915 | 1.5215 | 0.7565 | 0.6715 | 0.2675 | 0.0945 |
| DUSP23 | 0.5285 | 2.1905 | −0.5435 | −1.8165 | 0.2165 | −0.2345 | −0.3225 | −1.0605 | −0.8005 | 0.7555 |
| DUSP4 | 0.4965 | 0.0685 | −0.0185 | −0.4225 | 1.0355 | 0.2765 | −0.5415 | −0.9295 | 0.5445 | −0.1975 |
| DUSP5 | −0.006 | −0.426 | −0.196 | −0.114 | −0.215 | −0.328 | −0.317 | −0.249 | −0.156 | −0.327 |
| DVL1 | 0.065 | 0.16 | −0.163 | 0.223 | 0.722 | 0.008 | 0.267 | 0.288 | 0.093 | 0.258 |
| DXYS155 [symbol] | −0.28 | −0.532 | −0.718 | −0.85 | −0.714 | −0.64 | −0.396 | −0.462 | 0.847 | 0.226 |
| DYRK4 | −0.9195 | −0.0515 | −1.1225 | 0.9085 | −1.1255 | −1.0165 | −0.6145 | −0.1705 | −0.9265 | −0.6785 |
| o(y)2 | 0.037 | 0.015 | −0.077 | −0.151 | 0.025 | −0.201 | 0.342 | −0.204 | −0.236 | −0.121 |
| EAF2 | 0.9035 | −1.2535 | −0.1975 | −1.2865 | −1.4965 | −0.1445 | −0.7365 | −0.5915 | 0.0385 | 0.4085 |
| EAP30 | −0.2355 | −1.0965 | 0.1455 | 0.7065 | −1.4105 | −0.4075 | 0.4585 | 0.4945 | 0.3305 | −1.1355 |
| ECHDC1 | 1.1115 | 0.5855 | −0.1535 | 0.3645 | −0.4245 | −0.1655 | −0.5145 | −1.1925 | 0.5695 | 1.6595 |
| ECHDC3 | 0.7055 | 1.0116 | 1.1585 | 0.3885 | 1.8225 | 1.5765 | 0.4475 | 0.0285 | 0.8325 | −0.2835 |
| EEF1A2 | 1.078 | 0.81 | 0.408 | 1.16 | 0.61 | −0.035 | 0.1 | 0.178 | 0.068 | −0.145 |
| EFHC1 | −0.4455 | 0.8965 | 0.0775 | 0.7375 | −0.1325 | −0.1015 | 0.1875 | 0.0505 | 0.5575 | 0.2025 |
| EFNA4 | −0.1405 | −0.7816 | −0.5265 | −0.5875 | −0.7515 | −0.7085 | −0.7275 | −0.2545 | 0.4755 | 0.1645 |
| EFS | −0.5055 | 0.2035 | 0.1835 | 0.3895 | 0.1785 | 0.5005 | −0.2355 | −0.4465 | 0.1385 | −0.4055 |
| EIF2C2 | −0.0405 | −0.8255 | −0.6765 | −0.3095 | 0.3185 | −0.2455 | 0.1115 | −0.4035 | −0.2885 | −0.3205 |
| EIF2C4 | −0.02 | 0.256 | −0.059 | 0.286 | 0.417 | −0.045 | −0.078 | 0.007 | 0.272 | 0.409 |
| EIF4EBP1 | 0.7135 | 1.0335 | 0.0565 | 0.7155 | 0.8795 | 0.5075 | −0.1715 | −0.1875 | −0.0665 | 0.7425 |
| EIF4G1 | 0.074 | −0.287 | −0.314 | −0.611 | −1.127 | −0.351 | −0.008 | −0.418 | −0.628 | −0.179 |
| EIF4G3 | 1.401 | −0.728 | −0.422 | 0.031 | −0.094 | 1.263 | 0.537 | 1.759 | −1.471 | −0.303 |
| ELAC1 | −0.518 | −0.164 | 0.363 | 0.268 | −0.468 | −0.683 | −0.417 | 0.274 | 1.277 | −0.385 |
| ELL2 | 1.5645 | 2.5795 | 1.8135 | 0.3265 | 0.0045 | 1.7705 | 1.2515 | 1.0615 | 0.0425 | −0.2495 |
| ELL3 | 0.1665 | −0.6725 | −0.3125 | −0.7755 | 0.5875 | −0.0875 | −0.1915 | 0.1465 | −0.1845 | 0.1585 |
| ELOVL5 | 0.3915 | 1.3215 | 0.6035 | −1.0815 | −0.2665 | −0.5005 | −0.5835 | −0.1055 | −0.1875 | 0.1585 |
| EN1 | 1.4585 | 2.1905 | 0.9305 | 0.6965 | 1.5995 | 1.7245 | 1.4455 | 1.5085 | −1.2585 | −0.3855 |
| ENAH | 0.0935 | 0.4375 | 0.0495 | 0.7375 | −0.1125 | 0.2875 | 0.7025 | 0.0805 | 0.0395 | 1.6225 |
| EPHX2 | −0.104 | 0.428 | −0.205 | 0.513 | −0.119 | 0.027 | 0.025 | 0.312 | 1.181 | 0.726 |
| EPLIN | 0.6875 | 0.9855 | 0.9365 | −0.4325 | 1.3205 | 0.2595 | 0.0505 | −0.2835 | 2.5175 | 0.7155 |
| EPN3 | −0.8535 | 1.1745 | 0.7905 | −0.1785 | 1.2085 | 0.4195 | 0.9175 | 0.7545 | 0.4975 | −0.5355 |
| EPOR | −0.058 | −0.028 | −0.139 | −1.119 | −0.239 | 0.249 | 0.121 | −0.294 | −0.585 | 0.149 |
| ERBB2 | 2.2945 | 3.3925 | 1.6895 | 2.1035 | 2.5575 | 3.1885 | 2.8495 | 3.6715 | 2.8595 | 1.4495 |
| ERP70 | −0.888 | 1.291 | 0.056 | −0.051 | −0.614 | −0.84 | −0.316 | −0.821 | −0.086 | −0.901 |
| ESR1 | −0.5755 | 0.1765 | −0.0095 | 0.1975 | 0.7885 | 0.1395 | 0.2225 | 0.1415 | −0.1105 | 0.1585 |
| ETFA | 0.1576 | −0.8635 | −0.6305 | 0.4715 | 0.8925 | 0.3965 | 0.0105 | 0.3165 | −0.0755 | −0.1885 |
| ETV8 | 0.1165 | −0.0005 | 0.1845 | −1.2075 | −1.0965 | −1.6135 | −1.0475 | −0.9875 | −0.6725 | −0.2065 |
| EVER1 | −0.7595 | −0.4275 | −0.3615 | −0.5455 | −0.1105 | −0.3915 | 0.0585 | −0.2775 | −0.1495 | −0.0475 |
| EXO1 | −1.3855 | 0.2845 | −0.0805 | −0.4255 | −0.2875 | −0.2925 | 0.2215 | −0.1255 | 0.0355 | 0.1745 |
| EXOSC9 | −0.537 | −0.268 | 0.003 | −1.526 | −0.964 | −1.187 | −0.768 | −1.023 | 0.17 | −1.147 |
| EXT2 | 1.834 | 1.17 | 1.002 | 0.169 | 0.054 | 0.492 | 0.989 | 1.278 | 0.144 | −0.434 |
| EZH2 | −0.0875 | 0.3865 | 0.2165 | 0.0925 | −0.0075 | −0.1835 | −0.1865 | 0.0035 | −0.4305 | −0.9925 |
| FI1R | −0.607 | 0.619 | −0.349 | 0.964 | 0.561 | −0.207 | −0.194 | −0.039 | −0.774 | −1.917 |
| F9 | −0.125 | −0.38 | −1.042 | −1.427 | −1.567 | −1.036 | −0.598 | −1.938 | −0.878 | 0.128 |
| FA2H | −0.232 | −0.326 | −0.139 | 0.63 | −0.675 | −0.254 | −0.022 | 0.039 | 0.188 | −0.228 |
| FABP7 | 0.191 | 0.496 | 0.347 | 0.382 | −1.304 | −0.118 | 0.657 | 1.472 | 1.49 | 0.278 |
| FADD | −0.146 | −0.074 | −0.065 | 0.845 | 0.181 | 0.264 | 0.866 | 1.037 | −0.031 | −0.307 |
| FAHD1 | 1.027 | 0.43 | 0.09 | 0.934 | 0.378 | −0.014 | −0.31 | −0.448 | −1.051 | −0.828 |
| FAHD2A | 0.01 | −0.412 | −0.312 | 0.266 | 0.027 | −0.265 | 0.396 | −0.058 | 0.041 | 0.072 |
| FAM38A | −1.3225 | −0.0655 | −0.6875 | −0.4265 | −0.4045 | −0.9775 | 0.7945 | 0.2285 | 1.5515 | 1.4595 |
| FAM3A | −1.2125 | 0.1265 | −0.6395 | −1.1625 | −1.3055 | −0.7745 | −0.6695 | −1.1325 | −0.0115 | 0.3855 |
| FAM3B | 1.542 | 0.595 | −0.454 | 0.758 | 0.236 | 0.821 | 0.178 | 0.244 | −0.148 | 0.263 |
| FAM3C | −0.529 | −0.333 | −0.986 | −0.837 | −0.89 | −0.184 | −0.761 | 0.17 | 1.393 | −1.024 |
| FAM46C | −0.09 | −0.476 | −0.095 | −0.589 | −1.021 | −1.201 | −1.158 | −0.515 | −0.375 | −0.004 |
| FAM49B | −0.743 | −0.141 | −0.696 | −0.501 | −0.725 | −1.49 | −1.304 | −1.078 | −0.706 | −0.551 |
| FAM54A | −0.489 | −1.683 | −1.179 | −0.201 | −0.341 | −0.254 | −0.278 | −0.676 | 0.234 | 0.226 |
| FANCA | −0.2625 | −0.6575 | 0.3415 | −0.7395 | −0.1735 | −0.4025 | 0.4105 | −0.2545 | −0.2625 | −0.2185 |
| FANCE | 0.2735 | 0.7415 | 0.8815 | 1.0115 | −0.1745 | 1.1955 | 1.1355 | 1.3085 | 0.9445 | 0.3255 |
| FBL | 0.128 | −0.075 | 0.087 | −0.456 | 0.273 | 0.154 | 0.105 | 0.372 | 1.475 | 0.779 |
| FBP1 | 0.165 | −0.097 | −0.143 | 0.743 | −0.33 | −0.297 | 0.026 | 0.355 | −0.112 | −0.076 |
| FBS1 | 0.0315 | −0.0505 | 0.1335 | 0.9045 | 0.0725 | 0.2905 | 0.9375 | 0.6355 | −0.3165 | −0.2285 |
| FBXL20 | 0.54 | −0.075 | −0.269 | −0.276 | −0.092 | −0.085 | 0.094 | −0.114 | 0.411 | 0.84 |
| FBXO25 | −0.074 | −0.024 | −0.32 | −0.584 | −0.71 | −0.592 | −0.8 | −1.096 | 0.063 | 0.325 |
| FDXR | 0.089 | −0.169 | −1.006 | 2.078 | −0.238 | −0.956 | −0.638 | −1.047 | −0.209 | 0.487 |
| FEM1B | −0.485 | 1.093 | −0.258 | 1.075 | −0.293 | −0.758 | 0.308 | −1.115 | −2.188 | −0.304 |
| FGF12 | −0.0645 | 0.2155 | −0.3145 | −0.0005 | 1.2805 | 0.4675 | 0.0285 | −0.1525 | −0.1855 | 0.6435 |
| FGF13 | 0.339 | −0.407 | −0.164 | −1.431 | 0.268 | −0.191 | 0.95 | 0.586 | −1.157 | 0.274 |
| FHOD1 | −0.6285 | 1.0885 | 1.1965 | −0.8245 | −0.2075 | −0.3255 | −0.6325 | −0.8365 | 0.4685 | 0.4575 |
| FKBP11 | 0.8955 | 1.2115 | 1.1155 | 0.0805 | −1.4415 | 0.8555 | 0.4205 | 0.8365 | 0.6065 | −0.9955 |
| FLJ10094 | 0.371 | −0.216 | −0.501 | −1.341 | −0.333 | −0.498 | 0.352 | −1.576 | −0.465 | 1.222 |
| FLJ10115 | −0.0685 | −0.2385 | 0.2115 | −0.1485 | −0.7615 | −0.4195 | −0.0595 | −0.0505 | 0.3115 | 0.2875 |
| FLJ10159 | 0.107 | −0.038 | 0.347 | 0.488 | 0.637 | 0.409 | −0.16 | −0.832 | 0.45 | 0.307 |
| FLJ10204 | −0.479 | −0.147 | −0.077 | −0.049 | −0.448 | −0.652 | −0.506 | −0.109 | −0.285 | −0.18 |
| FLJ10275 | 1.0585 | −0.0665 | −1.1455 | −1.3195 | −0.3875 | 0.8415 | 0.0985 | −1.2575 | −1.0915 | −0.0105 |
| FLJ10292 | 0.5055 | −0.1955 | −0.0255 | −0.4715 | 0.3135 | 0.3625 | −0.1135 | 0.2675 | −0.3385 | −0.1455 |
| FLJ10324 | −0.2945 | −0.1845 | 0.5465 | −0.4635 | −0.6555 | −0.8375 | −0.2515 | −0.1455 | −0.2765 | −0.3725 |
| FLJ10700 | 0.306 | 0.243 | 0.564 | 1.081 | 0.535 | 0.323 | 0.999 | 0.773 | −0.288 | −0.125 |
| FLJ10706 | −0.601 | −1.016 | −1.129 | −0.937 | −1.032 | −2.351 | −0.791 | −1.017 | 0.038 | −0.425 |
| FLJ10671 | 0.121 | −0.286 | 0.242 | −0.091 | 1.01 | 0.842 | 1.139 | 1.357 | 1.289 | 0.794 |
| FLJ10901 | 1.3545 | 0.9055 | 1.7435 | 1.2295 | 1.2475 | 2.2455 | 0.6025 | 0.7005 | 0.7285 | −0.2985 |
| FLJ10918 | 0.373 | −1.443 | −1.543 | −0.274 | 0.219 | 1.393 | 1.038 | 1.486 | 0.29 | −0.402 |
| FLJ10980 | 0.3665 | −0.0335 | 0.0545 | −0.3905 | −0.9255 | −0.2175 | −0.3825 | −0.0285 | −0.0655 | −0.0515 |
| FLJ11017 | −0.0085 | 0.8005 | −0.9065 | 0.5255 | −1.0855 | −0.5895 | −0.8305 | −1.3545 | 1.2115 | 0.7185 |
| FLJ11088 | −0.1385 | −0.5155 | −0.5075 | −0.1895 | −0.0455 | −0.2785 | 0.0035 | −0.2215 | 1.1385 | 0.0015 |
| FLJ11151 | 1.2355 | 0.9515 | 1.0105 | 0.9195 | 0.5315 | 0.7425 | 0.9965 | 1.4495 | 0.4445 | 0.5635 |
| FLJ11267 | 0.1885 | 0.3475 | −0.0616 | 0.3705 | 0.0615 | 0.5375 | 0.2895 | −0.2025 | 1.0555 | 0.8265 |
| FLJ11280 | −0.6305 | 0.0385 | −0.2355 | 0.6435 | 0.4405 | −0.2555 | 0.2155 | −0.3885 | 0.0535 | 0.4265 |
| FLJ11508 | −0.0035 | −0.4755 | −0.4435 | 0.9145 | −0.6395 | −0.3085 | 0.4525 | 0.3165 | −0.0045 | −0.2035 |
| FLJ12270 | 0.9255 | −2.6845 | 0.1155 | −0.3635 | −1.0705 | −1.1865 | 0.8325 | 1.0815 | 3.2005 | −0.1876 |
| FLJ12844 | −0.3655 | −0.3735 | −0.1855 | −0.0785 | 0.0365 | −0.1535 | −0.1175 | −0.3385 | 0.4845 | 0.1835 |
| FLJ12650 | 0.1885 | −0.4445 | −0.0865 | −0.0275 | −0.0805 | −0.6375 | −0.2685 | −0.1715 | −0.4015 | −0.4995 |
| FLJ12684 | −0.2015 | −0.2515 | −0.0555 | −0.3015 | 0.6155 | 0.2705 | 0.2365 | 0.3925 | −0.1595 | 0.2455 |
| FLJ12735 | 0.447 | 0.46 | 0.411 | 1.768 | 1.807 | 1.263 | −0.801 | −0.38 | 0.9 | 0.231 |
| FLJ12750 | 0.169 | 0.921 | 1.199 | 0.072 | 1.593 | 1.89 | 0.616 | 1.039 | 1.7 | 1.738 |
| FLJ12895 | −0.7645 | −0.2505 | −0.1445 | −0.4275 | −0.0935 | −0.0775 | 0.1375 | −0.2705 | 0.0425 | −0.1125 |
| FLJ13710 | −0.5535 | 0.0605 | −0.3125 | −1.1065 | −0.9825 | −1.0285 | −0.8915 | −0.9135 | −0.6775 | −0.7125 |
| FLJ13855 | 0.269 | −0.647 | −0.586 | 0.134 | 0.419 | 0.018 | 0.347 | 0.247 | −0.109 | −0.123 |
| FLJ13912 | 0.018 | −0.386 | 0.296 | −0.157 | −0.401 | −0.833 | −0.495 | −0.395 | −0.458 | 0.064 |
| FLJ14124 | −0.286 | −0.895 | −0.28 | −0.318 | 0.263 | −0.032 | −0.263 | −0.126 | −0.039 | −0.604 |
| FLJ14627 | −0.1565 | 0.0355 | 0.1835 | 0.0045 | 0.0685 | 0.0295 | −0.1775 | −0.2655 | −0.3345 | −0.2405 |
| FLJ14666 | −0.342 | 0.443 | 0.17 | 1.207 | −0.964 | −0.408 | −0.18 | 0.318 | 0.162 | −0.05 |
| FLJ14904 | 1.418 | −0.887 | −1.76 | 2.168 | 0.206 | 1.242 | 2.02 | 2.372 | −0.057 | 1.783 |
| FLJ20035 | −0.608 | 0.187 | 0.098 | −0.478 | −0.005 | −0.163 | 0.166 | 0.664 | 0.971 | −0.001 |
| FLJ20152 | −0.243 | −0.632 | 0.117 | −0.293 | 0.695 | −0.331 | 0.203 | −0.027 | 0.038 | −0.086 |
| FLJ20171 | −0.052 | 1.628 | 1.439 | 1.028 | −0.147 | 0.743 | 0.676 | 0.951 | 0.738 | 0.081 |
| FLJ20244 | −0.896 | −1.129 | 0.241 | 1.699 | −0.651 | −0.106 | 0.11 | −0.384 | −0.182 | −0.111 |
| FLJ20273 | 0.221 | 0.819 | −0.082 | −0.115 | −0.008 | 0.225 | −0.181 | 0.031 | −0.395 | 0.141 |
| FLJ20315 | 0.705 | −0.579 | −0.458 | −0.627 | −1.043 | −0.877 | −0.864 | −0.72 | −0.242 | 0.135 |
| FLJ20323 | −0.1085 | 0.0785 | 0.2105 | −0.2275 | −0.1365 | −0.1875 | −0.2305 | −0.0125 | 0.6125 | −0.4495 |
| FLJ20518 | −0.074 | 0.129 | −0.12 | 0.11 | 0.548 | 0.473 | −0.035 | 0.309 | 0.258 | −0.046 |
| FLJ20530 | −0.326 | −0.243 | −0.604 | −0.199 | −0.235 | −0.977 | −0.748 | 0.413 | −0.702 | 0.003 |
| FLJ20696 | 0.0185 | −0.3815 | −0.5555 | 0.0485 | −0.6315 | −0.4745 | −0.0035 | 0.0685 | 1.4435 | 0.7185 |
| FLJ20718 | 0.265 | −0.093 | −0.295 | −0.029 | −0.285 | −0.237 | 0.055 | −0.346 | 0.448 | 0.186 |
| FLJ20772 | 0.4295 | −0.0675 | 0.3035 | 0.0545 | 1.8335 | 0.9075 | 0.7115 | 0.6385 | 0.8735 | 0.5455 |
| FLJ20989 | 0.3285 | 1.4435 | 2.6195 | 1.7425 | 2.0705 | 2.5275 | 1.8595 | 0.9435 | 0.3135 | 0.0475 |
| FLJ21019 | −0.7955 | 0.7395 | 1.1965 | −0.7755 | −1.4385 | −2.0335 | 0.1845 | −0.2055 | −0.0225 | 0.2035 |
| FLJ21062 | 0.7485 | 0.4785 | 0.1815 | −0.0985 | 0.3925 | 0.2245 | −0.0075 | 0.6585 | −0.1715 | −1.0575 |
| FLJ21159 | 0.9455 | 1.3295 | 1.1975 | 1.3715 | 1.2435 | 1.5845 | 1.1885 | 1.2985 | 0.8395 | 0.7205 |
| FLJ21616 | −0.009 | 0.766 | 0.185 | 0.362 | −1.617 | −0.178 | −0.472 | −1.121 | 0.543 | 1.74 |
| FLJ21827 | −0.0305 | 1.5445 | 0.4185 | 0.2055 | −0.6385 | 0.1516 | 0.3405 | 0.6735 | 0.5605 | 0.4916 |
| FLJ21963 | −0.039 | −0.601 | −0.151 | −0.374 | 0.074 | 0.366 | 0.131 | −0.185 | −0.333 | 0.052 |
| FLJ22104 | −0.1125 | −2.8165 | −0.9935 | 0.1875 | 0.3825 | 0.0175 | −0.3365 | −0.0585 | −0.8675 | −0.2815 |
| FLJ22222 | 0.285 | 0.364 | 0.459 | 0.108 | 1.48 | 0.721 | 0.644 | 0.828 | −0.048 | −0.558 |
| FLJ22573 | 0.4015 | 1.2535 | 0.0055 | 2.2445 | −0.4265 | −1.0925 | 1.7495 | 1.8535 | 0.7835 | 0.0385 |
| FLJ22794 | 0.371 | 0.099 | 0.603 | −0.307 | −0.551 | −0.3 | 0.343 | 0.057 | 0.343 | 0.166 |
| FLJ23188 | 0.7815 | 0.0055 | 0.4515 | −0.9965 | 2.3525 | −0.6075 | −0.1555 | 1.0735 | 0.9825 | 4.4345 |
| FLJ23441 | 0.927 | −0.268 | 0.331 | 0.218 | 0.767 | 0.144 | 0.505 | 0.094 | −0.121 | 0.046 |
| FLJ25471 | −0.38 | 0.909 | 0.158 | 0 | 0.299 | 0.111 | 0.402 | 0.66 | 0.267 | 0.089 |
| FLJ31204 | 0.5175 | −0.8995 | −1.1985 | −0.7745 | −0.7225 | −0.0105 | −1.1115 | −1.1275 | −0.1445 | 0.5705 |
| FLJ31795 | −0.5665 | −0.4315 | −0.2835 | −0.6195 | 0.7525 | 0.3755 | −0.1665 | −0.2355 | −1.0265 | −0.1045 |
| FLJ32942 | 1.1855 | 1.1555 | 0.5055 | 2.0155 | 0.1465 | 2.0305 | 1.3155 | 2.5525 | 1.6055 | 0.5515 |
| FLJ37970 | −1.297 | 0.72 | 0.531 | 0.514 | −4.021 | −2.624 | 0.117 | −1.558 | 2.282 | −0.608 |
| FLJ39370 | −0.8125 | −0.1315 | 0.3035 | 0.0115 | −0.2555 | −0.0395 | 0.2025 | 0.2425 | −0.1635 | 0.5095 |
| FLJ90588 | 0.2025 | −1.4235 | −0.3945 | −0.7845 | −0.5165 | −0.6165 | −0.1885 | −1.5475 | 0.1735 | 0.2265 |
| FN3KRP | 0.644 | 1.175 | 0.495 | 1.53 | 0.987 | 1.467 | 0.526 | 1.18 | 0.951 | −0.706 |
| FOLR1 | 0.9945 | −1.5485 | −1.0495 | −1.6795 | −0.5895 | −0.6285 | −0.0095 | −1.8395 | −0.9895 | 0.1105 |
| FOXA1 | 0.9115 | −0.1045 | −0.2475 | 0.3905 | 1.0615 | 0.8685 | 0.8625 | 0.7925 | 0.1235 | 0.5505 |
| FOXC1 | −0.946 | −0.034 | −0.19 | −0.782 | −0.945 | −1.547 | −0.552 | −1.071 | 0.051 | 0.699 |
| FOXP1 | −0.3965 | 1.0535 | 0.3465 | 2.0175 | 1.3945 | 0.2405 | 0.5985 | 0.0055 | −0.4775 | −0.0276 |
| FREQ | 0.2045 | 0.5825 | −0.0005 | 0.2495 | −0.6925 | −0.0145 | −0.3485 | 0.1275 | 0.2855 | 0.0635 |
| FTO | 0.9155 | 0.8195 | −0.0785 | 1.4675 | 0.4335 | 1.5515 | 0.3555 | 0.7025 | −0.1455 | 0.6175 |
| FUBP1 | 1.1025 | 2.4085 | 1.3365 | 1.8585 | 0.3455 | 1.2955 | 0.8725 | 1.0295 | 0.5075 | −0.3105 |
| FUCA1 | −0.893 | −0.127 | −0.724 | −1.011 | −0.668 | −0.509 | 0.115 | −0.514 | −0.821 | 0.227 |
| FUT8 | 0.4845 | 0.8575 | 0.1805 | −0.1745 | 0.3625 | 0.4575 | 0.4525 | 0.3216 | 0.4205 | 0.1455 |
| FXYD6 | 0.023 | −0.965 | 0.439 | −1.248 | 1.843 | 1.801 | −0.028 | −0.962 | −1.336 | 0.828 |
| FYCO1 | 0 | 0.207 | −0.362 | −0.992 | −1.604 | −1.862 | −0.176 | −0.82 | −0.402 | −0.131 |
| FZD10 | 1.0875 | −0.6705 | −0.5175 | 0.0485 | −0.8615 | −0.8755 | −0.7855 | −0.7815 | 0.3835 | 0.2276 |
| FZD7 | 0.5355 | −1.1755 | −0.7005 | 2.0075 | 0.1095 | −0.3715 | −0.5805 | −0.4055 | −0.2065 | 0.0835 |
| FZD9 | 1.6755 | −0.7785 | −0.7195 | 3.3735 | −0.5815 | −0.5275 | −0.9805 | −0.4325 | −0.4675 | −0.0405 |
| G1P2 | −0.2185 | −0.1475 | −0.0115 | −0.7185 | −0.3745 | −0.6465 | −0.2955 | −0.0665 | −0.4875 | −0.8055 |
| G1P3 | −0.38 | 0.154 | −0.425 | 0.558 | −1.232 | −0.682 | −0.535 | −0.344 | −0.095 | −0.034 |
| GA17 | 0.0775 | −1.3255 | −0.1395 | −1.0155 | −1.9025 | −1.7975 | −1.9135 | −1.6485 | −0.9545 | −0.8795 |
| GABARAP | 0.7255 | 0.3105 | 0.2055 | 1.3045 | 1.5555 | 0.8075 | −0.2285 | 0.1415 | 0.6975 | 0.1715 |
| GAJ | −0.3605 | −1.0735 | −0.8025 | 1.4135 | −0.1955 | −0.4005 | −0.4715 | −1.6035 | −1.3885 | −1.2735 |
| GALE | 1.091 | 0.343 | −0.186 | −0.053 | 0.795 | 0.154 | 0.772 | 1.007 | −0.288 | −0.407 |
| GALNT14 | 1.3775 | 2.2845 | 1.0876 | 0.5685 | 1.5435 | 1.6645 | 1.5515 | 2.5255 | −0.1315 | −1.5555 |
| GALNT2 | 1.3585 | 0.8345 | 0.1975 | 1.2835 | 1.0855 | 0.4815 | 0.5945 | 0.4785 | 0.5935 | 0.3575 |
| GALNT7 | −0.308 | −0.273 | −0.261 | −0.557 | −0.526 | −0.772 | −0.074 | −0.034 | 0.091 | −0.4 |
| GAMT | −0.557 | −0.434 | −0.521 | −0.718 | 0.006 | −0.58 | −0.178 | −0.02 | −0.471 | 0.761 |
| GAPD | −0.091 | 0.419 | 0.267 | −0.442 | 0.162 | −0.121 | 0.341 | 0.141 | −0.074 | −0.013 |
| GARS | 1.102 | 1.082 | 0.985 | 1.801 | 1.375 | 1.067 | 0.83 | 1.377 | 0.77 | −0.004 |
| GART | 0.1585 | 0.2225 | 1.4715 | 0.4125 | 0.8355 | 1.5535 | 0.9515 | 1.2105 | 1.2085 | −0.6515 |
| GATA3 | 0.6895 | 0.0065 | 0.4275 | −0.5315 | 0.8125 | −0.2835 | 1.0615 | 0.5865 | −0.2965 | −0.3375 |
| GATM | 0.223 | 0.794 | 0.485 | 1.076 | −0.157 | 0.648 | 0.84 | 0.186 | 0.174 | 3.9 |
| GBA | −0.94 | 0.142 | −0.111 | −0.633 | −1.087 | −0.459 | −0.279 | −0.216 | −0.265 | 0.377 |
| GDAP1 | 0.285 | −0.05 | 0.183 | −0.81 | 0.524 | 0.37 | 0.011 | −0.264 | −0.324 | 0.234 |
| GDI2 | 1.0535 | 0.1255 | 0.0825 | 0.4985 | 0.1275 | 0.7185 | 0.8735 | 1.0665 | 0.1245 | −0.2665 |
| GGA1 | 1.5535 | 1.0465 | 0.6185 | 0.5085 | 0.3385 | 0.0775 | 0.5495 | 0.7885 | 0.8515 | 0.3525 |
| GKAP1 | −0.6655 | 0.1505 | −0.4825 | −0.4945 | −0.0335 | −0.4205 | 0.3065 | −0.4265 | −0.2115 | 0.6465 |
| GLCCI1 | 0.5415 | −0.3895 | 0.9225 | −0.9415 | 0.2445 | 0.3645 | 0.3885 | 0.0475 | −0.5335 | −0.1895 |
| GLG1 | −0.258 | −0.309 | 0.474 | −0.916 | −0.563 | −0.815 | −1.109 | −0.961 | −0.386 | 0.093 |
| GLTSCR2 | 0.352 | 1.082 | 0.406 | 0.767 | 1.231 | 0.857 | 1.913 | 2.078 | 1.41 | 0.233 |
| GNB4 | −0.23 | 0.103 | 0.271 | 0.497 | 0.112 | −0.162 | 0.004 | 0.148 | −0.014 | −0.367 |
| GOLPH2 | −0.0365 | −0.4925 | −0.2005 | −0.1835 | 0.5405 | −0.2885 | −0.8755 | −0.9795 | −0.3245 | −0.0735 |
| GPATC1 | −0.5816 | −0.3965 | −0.0705 | −1.3195 | −0.3385 | −0.6785 | 0.0555 | −0.2105 | −0.2275 | −0.4285 |
| GPC2 | −0.8816 | 1.0455 | 0.7655 | 1.0985 | −0.7265 | 0.8165 | 0.7125 | 1.1225 | 0.3335 | −0.6065 |
| GPR125 | −0.841 | −0.37 | −1.072 | −0.466 | −0.686 | −0.4 | −0.827 | −0.957 | −0.425 | −0.24 |
| GPR160 | 0.0895 | −0.5195 | −1.2975 | 0.4585 | 1.0365 | 0.7045 | 0.2465 | 0.2155 | −0.4255 | −0.0665 |
| GPR56 | −0.0145 | −1.1485 | −0.3895 | −0.4035 | 0.3325 | −0.3555 | −0.1995 | −0.3635 | −0.8035 | 0.8475 |
| GPRC5C | 0.3615 | 0.0055 | −0.0055 | −0.9615 | −1.1735 | −1.0675 | −1.0405 | −0.8245 | −0.0335 | −0.1035 |
| GPSM1 | 0.594 | 0.508 | 1.409 | 0.07 | 0.568 | 0.508 | 0.482 | −0.012 | −0.535 | 0.716 |
| GPSM2 | 0.147 | 1.364 | −1.118 | −1.467 | 1.491 | −0.32 | −1.049 | −1.056 | 0.208 | −0.132 |
| GPT | −1.0185 | 0.2315 | −0.5215 | −1.2455 | 0.4185 | −0.4375 | 0.0555 | −0.5265 | −0.0945 | 1.4655 |
| GPT2 | 0.905 | −1.554 | −1.707 | 5.104 | 6.974 | −1.29 | −0.57 | −0.695 | 0.242 | −0.203 |
| GPX7 | −0.358 | −0.231 | −0.089 | 0.656 | 0.108 | −0.177 | −0.186 | −0.059 | 0.043 | −0.196 |
| GRB14 | 1.9155 | 2.3545 | 2.7565 | −0.0065 | 2.1865 | 1.5965 | −0.2125 | −1.7805 | −0.9235 | −0.0675 |
| GRB7 | −0.5455 | −0.8355 | −0.3005 | 1.2365 | 0.1235 | −0.3535 | −0.1085 | −0.4015 | −0.0435 | 0.1175 |
| GSC | −0.268 | 0.683 | −0.042 | 0.615 | 0.248 | −0.097 | 0.151 | 0.307 | 0.008 | 0.131 |
| GSDML | −0.409 | −0.01 | −0.205 | 0.12 | −0.363 | −0.419 | −0.465 | −0.478 | −0.127 | −0.431 |
| GSK3B | 0.324 | 0.37 | 0.41 | 0.48 | 0.136 | 0.148 | 0.344 | −0.299 | −0.044 | −0.229 |
| GSTA1 | 1.0485 | 0.3095 | 0.1215 | 3.0005 | 0.2725 | 1.3405 | −0.1855 | −0.4015 | 2.3915 | 0.1065 |
| GSTK1 | −0.246 | 1.003 | −1.073 | −1.998 | 0.918 | 1.481 | 1.306 | 1.315 | −1.41 | 0.201 |
| GSTM3 | 0.5325 | 0.2585 | 0.8215 | 1.0465 | 0.3415 | 1.1785 | 0.8625 | 0.7535 | −1.7375 | −0.0545 |
| GSTP1 | −0.419 | −0.98 | −0.695 | −0.634 | −0.28 | −0.046 | 0.632 | −0.442 | 0.556 | 0.735 |
| GSTT1 | −0.1945 | 0.3475 | 0.0825 | −0.7075 | −0.2945 | −0.2715 | −0.3475 | −0.0065 | −0.0195 | −0.2695 |
| GSTT2 | −0.0995 | 0.1935 | 0.2805 | −0.5225 | −0.1475 | −0.0485 | 0.1775 | −0.0525 | 0.6845 | −0.6785 |
| GTF2F2 | −0.478 | 0.015 | 0.056 | −1.426 | −0.212 | 0.304 | 0.043 | 0.207 | 0.535 | 0.607 |
| GTF2I | −0.0065 | −0.5565 | 0.1805 | 0.4175 | 1.0265 | 0.0285 | 0.2795 | 0.0365 | 0.1785 | 0.1185 |
| GTF3A | −0.891 | −0.097 | −0.019 | −0.724 | −0.88 | −0.6 | −0.315 | −0.271 | 0.138 | −0.654 |
| GTPBP3 | 1.69 | 0.853 | 1.238 | 0.66 | 1.396 | 1.878 | 1.475 | 1.407 | 1.545 | 0.092 |
| GTPBP4 | −0.266 | −0.339 | −0.135 | −0.307 | −0.738 | −0.367 | −0.327 | 0.46 | −0.281 | −0.871 |
| H1F0 | −0.3945 | 0.2975 | −0.0635 | 0.1495 | 0.3555 | 0.1295 | 0.4215 | 0.3955 | 0.3485 | 0.8305 |
| H2AFV | −0.3865 | −0.6445 | −0.2095 | −0.1075 | −1.4615 | −0.8885 | −0.5245 | −0.0815 | −0.3605 | −0.8775 |
| H2AFY | 0.1195 | 1.2425 | −0.9215 | −0.4465 | −0.3465 | −0.9075 | −0.8765 | −0.5145 | −0.4455 | 0.5045 |
| H2AFZ | −0.4775 | −0.5205 | −0.2965 | −0.3025 | −0.1985 | 0.6385 | −0.0615 | −0.8855 | 0.2875 | 0.0185 |
| H2BFS | −0.2805 | −0.8315 | 0.2665 | 2.4945 | −0.0985 | −0.4075 | −0.1055 | −0.2665 | 0.2385 | −0.0335 |
| H3F3B | 0.623 | −0.012 | 0.231 | 1.185 | 0.554 | 0.498 | 0.372 | 0.8 | 1.34 | 0.947 |
| H41 | 0.7195 | −0.4525 | 0.3005 | 0.1715 | 1.0465 | 0.4015 | 1.2035 | 0.0495 | 0.4295 | 0.6585 |
| HAGH | 0.155 | 0.851 | 0.133 | 0.711 | −0.371 | 0.015 | 0.435 | 0.529 | 0.391 | −1.036 |
| HBG1 | −0.3975 | −0.8505 | 0.2385 | −2.0535 | −0.8495 | −1.5345 | −0.0995 | −0.8295 | −0.3435 | −1.7035 |
| HBL01 | 0.046 | 0.453 | −0.458 | −0.686 | −0.292 | −0.244 | −0.683 | −0.057 | −0.31 | −0.345 |
| HCAP-G | −0.4845 | −0.1405 | 0.0035 | −0.2875 | −0.4915 | −0.0775 | −0.1885 | −0.2115 | −0.0385 | −0.3995 |
| HCP5 | 0 | 0.203 | −0.563 | −0.183 | −0.388 | −0.46 | 0.406 | −1.343 | 1.365 | 0.993 |
| HDAC2 | 0.367 | 0.305 | 0.543 | 0.336 | −0.772 | 0.338 | 0.447 | 1.119 | 1.56 | 0.641 |
| HDC | −0.1075 | −0.0505 | −0.0565 | 0.9185 | 0.0175 | −0.0575 | −0.1115 | −0.0335 | −0.3295 | −0.7825 |
| HEBP1 | 0.714 | 0.617 | 1.41 | −0.912 | −1.427 | −1.902 | 1.159 | 0.151 | −0.885 | 1.086 |
| HIPK2 | 1.258 | 0.793 | 0.121 | 0.891 | 0.685 | 0.71 | −0.094 | 1.058 | 1.345 | 0.605 |
| HIPK2 | 1.0485 | 1.0085 | −0.4695 | 0.5915 | 0.2205 | −0.5265 | −0.3985 | −0.4345 | 0.0035 | 0.2745 |
| HIS1 | 0.4995 | 1.4105 | −0.7855 | 0.3885 | 0.2325 | −0.4545 | −0.5065 | −0.3195 | −0.4395 | 0.6615 |
| HIST1H2A | 0.511 | 1.409 | −0.776 | 0.266 | 0.283 | −0.433 | −0.473 | −0.348 | −0.413 | 0.62 |
| HIST1H2B | 0.3325 | 1.2295 | −0.6415 | −0.0425 | 0.2495 | −0.4455 | −0.3275 | −0.3725 | −0.4716 | 0.3035 |
| HIST1H2B | 0.51 | 1.352 | −0.498 | 0.051 | 0.289 | −0.224 | −0.32 | −0.295 | −0.337 | 0.637 |
| HIST1H2B | −0.3135 | −0.3595 | −0.4315 | 0.1205 | −0.6515 | 0.3135 | −0.4045 | −0.9335 | 0.2135 | 0.2745 |
| HIST1H2B | 0.0505 | −0.5875 | −0.0415 | −0.8495 | 0.1555 | −0.3535 | 0.3955 | 0.0885 | −0.2065 | −0.1205 |
| HIST1H3C | −0.9605 | 0.0565 | −0.4725 | 1.9305 | −0.3105 | −0.4745 | −0.2905 | −0.9335 | −0.4685 | −0.9255 |
| HM13 | −0.7955 | −0.2575 | 0.0255 | −0.6035 | −0.3695 | −1.2765 | −0.7445 | −0.4625 | −0.7045 | −1.4225 |
| HMGB3 | −0.1225 | 0.0485 | 0.0375 | −1.1285 | −0.4745 | −0.2035 | −0.1045 | 0.0645 | 0.3565 | −0.2415 |
| HN1 | −0.383 | −0.098 | −0.084 | −0.779 | −0.672 | 0.023 | −0.093 | 0.181 | 0.152 | 0.105 |
| HNRPA3P | 0.3055 | 0.1255 | −0.0486 | 0.7415 | 0.2845 | −0.1415 | 0.8595 | 0.3635 | −0.6885 | 0.1645 |
| HNRPDL | 0.64 | 0.188 | −0.098 | −0.908 | 0.662 | 0.035 | 0.943 | −0.875 | −0.358 | 1.138 |
| HOXA5 | 0.9235 | 1.3415 | 0.2115 | 1.6445 | 1.6875 | 1.2895 | 1.5705 | 1.7205 | −1.3265 | 0.2085 |
| HOXA7 | −0.3235 | 0.6895 | −0.4505 | 3.8935 | 3.6115 | −0.6165 | 3.5875 | 4.5955 | −0.1385 | −0.5185 |
| HOXB2 | 0.5135 | 0.2055 | −0.8345 | 1.8495 | 1.5005 | −0.7435 | 2.2105 | 1.7905 | −0.5795 | 0.9825 |
| HOXB6 | 2.0765 | −0.5645 | 1.5085 | −0.1265 | 2.1445 | −1.0515 | −0.1675 | −1.6825 | −1.0025 | −0.3525 |
| HOXB7 | 1.3695 | −0.1345 | 1.3375 | 0.1235 | −0.2085 | −1.1285 | −0.2105 | −1.0595 | 0.4605 | 1.1625 |
| HOXC10 | −0.426 | −0.397 | 0.88 | 0.75 | 0.948 | 0.213 | −0.12 | −0.523 | −0.308 | −0.474 |
| HOXC13 | 0.3525 | −0.0275 | 1.2575 | 0.0435 | 0.7015 | 0.5205 | 0.5855 | 0.2405 | −0.0175 | 0.5975 |
| HOXC9 | 0.426 | 0.376 | 0.284 | −0.663 | −0.072 | 0.334 | 0.27 | 0.098 | 0.581 | 0.379 |
| HOXD3 | 0.0485 | 0.1585 | −0.1705 | 0.9645 | −0.7255 | 0.1175 | −0.3225 | 0.1755 | −0.0645 | −0.0035 |
| HPCL2 | 0.34 | 0.378 | −1.354 | −1.273 | −1.01 | −1.947 | −2.204 | −2.738 | −1.548 | −1.357 |
| HPS3 | 0.0075 | 0.5005 | 0.1385 | 0.1065 | −1.1815 | −0.3455 | −0.0395 | 0.3515 | 0.2845 | −0.0355 |
| HRASLS | −0.0805 | 0.2685 | −0.4825 | −0.9975 | −1.0755 | −0.7765 | −0.0455 | 0.4075 | 0.4105 | −0.1175 |
| HRB | 0.264 | 2.473 | −0.837 | −1.858 | −0.938 | −1.096 | −0.851 | −1.698 | −1.872 | 2.004 |
| HRSP12 | −0.357 | −0.79 | −0.158 | −1 | −0.051 | −0.259 | −0.152 | −0.214 | 1.042 | 0.568 |
| HSD17B2 | −0.3085 | −0.0885 | −0.1845 | −0.4605 | 0.0435 | −0.2315 | −0.0705 | 0.2395 | −0.0495 | −0.1785 |
| HSF1 | 0.4365 | 0.1625 | 1.8235 | 0.4145 | 1.3435 | 1.0795 | 1.2245 | 1.2665 | 1.2855 | 1.8175 |
| HSPA14 | −1.707 | −0.36 | −1.387 | −0.56 | −0.454 | −1.436 | −0.254 | −0.244 | 0.109 | −1.05 |
| HSPA2 | 0.4485 | 0.0835 | 0.1535 | 0.5635 | 0.6865 | 0.4036 | 0.1535 | 0.2485 | 0.2855 | 0.1415 |
| HSPA5BP [symbol] | 0.026 | 0.272 | −0.185 | 0.768 | −0.86 | −0.229 | −0.025 | −0.116 | −0.127 | −0.113 |
| HSPC016 | 0.186 | 0.145 | −0.001 | −0.223 | 0.308 | 0.091 | 0.413 | −0.005 | −0.237 | −0.344 |
| HSPC138 | −0.55 | −0.499 | −0.666 | −0.97 | −1.53 | −2.068 | −0.502 | −0.268 | −0.822 | −1.852 |
| HSPC148 | −0.9795 | 0.0715 | 0.1915 | −0.7385 | −1.0145 | −2.6495 | −0.2855 | −0.1035 | −0.5715 | −1.1845 |
| HSPC150 | −0.2185 | −0.5665 | 0.0155 | −0.5735 | −0.2485 | 0.4445 | 0.1245 | −0.3635 | −0.0365 | −0.3825 |
| HSPC163 | 0.058 | −0.357 | −0.222 | −0.814 | −0.838 | −1.319 | −0.298 | −1.038 | −0.317 | −0.376 |
| HSPC266 | −0.516 | −0.321 | −0.378 | −0.759 | −1.063 | −1.081 | −0.405 | −0.762 | −1.187 | −1.459 |
| HSU79274 | −0.026 | −0.052 | −0.25 | −0.575 | −0.341 | −0.072 | −0.805 | −1.096 | 0.525 | 0.214 |
| HTATIP2 | −0.289 | 0.038 | 0.171 | −0.303 | −0.22 | −0.081 | 9.04 | 0.321 | −0.006 | −0.916 |
| ICMT | −0.364 | −1.361 | −0.623 | −1.666 | −1.371 | −1.19 | 0.503 | −1.825 | −1.079 | 0.338 |
| ICT1 | −0.879 | 0.53 | 0.384 | 0.254 | 0.104 | −0.204 | −0.614 | −0.777 | −0.046 | −0.121 |
| ID4 | 0.3285 | 0.5325 | −0.0855 | −0.2135 | −0.2905 | 0.0605 | −0.4735 | −0.6625 | −0.2475 | 0.4515 |
| IDH2 | −0.647 | −0.659 | −0.298 | 0.033 | 0.489 | −0.439 | −0.789 | −0.208 | −0.391 | −0.56 |
| IDS | −0.5995 | −0.1745 | −1.1075 | 1.1985 | −0.0235 | −0.4615 | −0.6395 | −0.8915 | −1.0415 | 0.2185 |
| IFI30 | 0.0335 | −0.0535 | −0.2005 | 0.5325 | −0.6645 | −0.1005 | −0.1965 | 0.3015 | −0.6075 | 0.0285 |
| IFI44 | −0.3675 | −0.4725 | 0.0335 | 0.7895 | −0.3055 | −0.4275 | −0.0175 | −0.2585 | 0.7175 | 0.3895 |
| IFIT5 | 0.0945 | 0.2635 | 0.4615 | 0.5495 | −0.0915 | 0.8925 | 0.1985 | 0.0455 | −0.1575 | −0.2645 |
| IFITM1 | 1.8015 | 1.7385 | 3.8745 | 0.5375 | 0.8025 | 1.9125 | 3.0445 | 3.9545 | 1.9505 | 0.9825 |
| IGBP1 | 1.1275 | 0.2715 | 0.7025 | 2.1205 | −0.5745 | 0.1745 | 1.2575 | 1.8185 | 1.7095 | −0.1235 |
| IGFALS | −0.7545 | −0.3755 | −0.6635 | −0.2195 | −0.0785 | −0.3975 | 0.2405 | 0.3325 | −0.7195 | −0.7865 |
| IGPBP2 | −0.8485 | 0.1385 | −0.3325 | −0.2355 | −0.5005 | −0.1825 | −0.1585 | 0.6505 | −0.6025 | −2.6105 |
| IGHG1 | −2.0335 | −0.6905 | −1.8405 | −1.0125 | −1.5775 | −2.2635 | 1.0585 | −0.0605 | −1.8785 | −2.9505 |
| IGHG1 | −1.379 | 1.266 | −1.356 | 0.135 | −1.333 | −1.245 | −0.231 | 1.511 | −0.961 | −1.508 |
| IGJ | −2.223 | 0.209 | −1.26 | 0.088 | −2.12 | −2.284 | 0.067 | 0.493 | −1.572 | −2.538 |
| IGKC | −0.371 | −0.223 | −0.781 | −0.114 | −1.009 | −0.86 | −0.254 | 0.489 | −0.752 | −0.746 |
| IGKC | −1.278 | −0.85 | −1.244 | 0.1 | −0.642 | −0.888 | 0.083 | 0.154 | −1.077 | −1.444 |
| IGLC2 | −0.2585 | 0.8555 | 9.2815 | 0.0455 | 0.3405 | 0.2215 | 0.5315 | 0.2415 | −0.3205 | 0.0095 |
| IGLL1 | 0.2035 | −0.0705 | −0.0465 | 0.0875 | −0.1435 | −0.0875 | 0.4885 | −0.7025 | −0.0875 | −0.2755 |
| IL13RA1 | −0.02 | −0.862 | −0.467 | −0.074 | −0.488 | −0.162 | −0.726 | −0.434 | −0.351 | −0.014 |
| IL17B | 1.8225 | 1.1625 | 1.0405 | 2.2185 | 0.8615 | 1.6425 | 2.2295 | 2.9495 | 0.3705 | −0.4305 |
| IL23A | −0.1375 | 0.3255 | 0.0035 | −0.1885 | −1.1185 | −0.6865 | −0.2015 | −0.1055 | −0.5025 | −1.0375 |
| IL6ST | −0.5055 | −0.4355 | 0.0935 | −0.9075 | −1.0005 | −1.2325 | −0.3405 | −0.9635 | 0.0175 | −0.4115 |
| ILF2 | 0.242 | 0.838 | 0.855 | 1.389 | −0.499 | 0.092 | 0.448 | 0.759 | 0.476 | −0.774 |
| IMPA2 | 0.2785 | 1.5285 | −0.0625 | −1.8735 | 0.1145 | 0.5545 | 0.4935 | −0.5635 | 1.7875 | 0.5255 |
| ING1L | 1.281 | 0.778 | 0.081 | 1.131 | 1.912 | 0.728 | 0.705 | 0.829 | 1.199 | 0.275 |
| INHBB | 2.4165 | −0.3085 | −0.0085 | −0.5825 | −0.2676 | −0.1655 | −0.0935 | −0.2385 | −0.4075 | −0.0175 |
| INPP4B | 0.7635 | 0.3125 | −0.5165 | 0.2005 | 2.0175 | 0.3305 | 1.5285 | 1.3645 | 2.8505 | 0.5615 |
| INSM1 | −0.124 | −0.012 | 0.906 | −1.312 | 0.534 | 0.407 | −0.27 | 0.728 | 0.995 | −0.174 |
| IQCA | −0.311 | 2.291 | 0.932 | 1.993 | 0.603 | 1.774 | 2 | 2.106 | −1.888 | −0.551 |
| IQCE | 0.341 | −0.846 | −0.559 | −0.549 | −0.032 | −0.637 | −0.35 | −0.594 | −0.637 | −0.238 |
| IQGAP2 | −0.9385 | −0.7355 | −0.0815 | −0.7775 | −0.1335 | −0.8005 | −0.4875 | 1.1315 | 0.2735 | −0.6545 |
| IRF4 | 0.0335 | −0.7775 | −1.1155 | −1.6435 | −1.7855 | −1.1085 | −0.2965 | −3.6645 | −1.1935 | −1.4315 |
| IRTA2 | −0.565 | −0.807 | −1.006 | 0.308 | 0.101 | −0.135 | −0.888 | −0.733 | −1.387 | −0.75 |
| IRX4 | −0.0475 | 0.0105 | −0.5735 | 1.6245 | 0.2325 | 0.0285 | −0.3935 | 0.1475 | 0.3825 | 0.1615 |
| ISG20 | −0.239 | −0.881 | −0.093 | −1.854 | 1.484 | 0.924 | −0.245 | −0.409 | 0.11 | −0.569 |
| ISGF3 [symbol] | 0.824 | 1.255 | −0.11 | 1.858 | −1.151 | 0.389 | 0.838 | 2.05 | −0.258 | 0.868 |
| ITGB4 | −0.098 | −0.22 | −0.016 | −0.425 | 0.436 | 0.102 | 0.62 | 0.277 | −0.101 | 0.353 |
| ITPR1 | 1.616 | 0.742 | 1.808 | 0.704 | 0.959 | 0.641 | 1.381 | 2.079 | 1.132 | −0.112 |
| JARID1A | −0.447 | −0.126 | −0.072 | −0.469 | −0.604 | −0.276 | −0.056 | −0.348 | 0.21 | −0.333 |
| KARCA1 | −0.195 | −0.441 | −0.257 | −0.624 | 0.032 | 0.399 | −0.131 | −0.203 | 0.047 | 0.624 |
| KATNA1 | −0.0095 | −0.0955 | 1.2795 | 0.1145 | 0.3735 | −0.1575 | 0.1825 | −0.8935 | 0.1335 | 1.0525 |
| KCNC2 | −0.188 | −0.311 | −0.048 | 0.895 | 0.311 | −0.145 | −0.392 | −0.12 | 0.014 | −0.26 |
| KCND2 | −0.952 | −0.651 | −0.402 | −1.412 | −0.02 | −0.98 | −0.926 | −1.456 | 0.391 | −0.33 |
| KCNK4 | 0.62 | 1.468 | 0.81 | −1.111 | 0.427 | 1.23 | 0.141 | 0.98 | 0.243 | 0.608 |
| KCNN4 | 0.0315 | 1.8435 | 0.6855 | 1.2045 | 1.0875 | 0.4265 | 1.0985 | 0.7205 | −0.0765 | −1.1435 |
| KCNS3 | 1.1785 | 1.0405 | 1.4615 | −0.5615 | 0.7795 | 1.0355 | 0.7705 | 1.3435 | 0.7155 | −0.2185 |
| KCTD15 | −1.174 | 0.204 | −0.124 | −1.222 | 0.011 | 0.281 | 0.086 | 0.91 | −0.583 | 0.42 |
| KCTD3 | −0.605 | −0.231 | 0.368 | −0.882 | −0.428 | −0.803 | 0.208 | 0.238 | −0.301 | 0.149 |
| KDELR2 | −0.515 | −0.512 | 1.053 | −1.701 | −0.845 | −0.789 | −1.5 | −0.734 | −0.002 | −1.357 |
| KIAA0020 | 0.168 | −0.005 | 1.217 | −0.45 | −0.524 | −0.165 | −0.01 | −0.505 | 0.037 | 0.045 |
| KIAA0101 | 0.0265 | 1.2125 | 0.0655 | 0.8015 | 1.3555 | 0.5075 | 0.0025 | −0.0315 | 1.0015 | 0.2555 |
| KIAA0153 | −0.0125 | −0.0985 | −0.2078 | −1.2845 | −0.7875 | 0.1275 | 0.1425 | 0.6285 | 1.2725 | 0.5845 |
| KIAA0182 | −0.4585 | −0.7165 | 0.4215 | −0.1805 | 0.3485 | −0.0145 | 0.0315 | 0.2035 | −0.1525 | −0.3745 |
| KIAA0196 | 0.0915 | 0.8565 | 0.2325 | 0.3885 | 0.8505 | 0.9115 | 0.4555 | 0.9045 | 1.1845 | 0.6985 |
| KIAA0241 | −0.21 | 0.214 | 0.395 | −0.544 | −0.306 | −0.058 | 0.434 | 0.646 | 1.287 | −0.454 |
| KIAA0251 | −0.0125 | −0.5385 | 0.2695 | −0.9035 | 0.4395 | 0.2685 | 0.0925 | 0.3925 | −0.2715 | −0.0125 |
| KIAA0274 | −0.065 | −0.249 | −0.069 | −0.476 | 0.14 | 0.288 | 0.925 | 0.215 | 0.248 | −0.204 |
| KIAA0515 | 0.0355 | −0.1775 | 0.2345 | −0.4785 | −0.2655 | 0.0755 | −0.3775 | −0.0415 | 0.0025 | −0.4825 |
| KIAA0582 | −0.0865 | 1.0745 | −0.9685 | 2.4095 | 1.5245 | 1.4185 | −0.1105 | 0.0595 | −0.9285 | −1.1735 |
| KIAA0650 | 1.0375 | 0.0875 | −0.4885 | −0.2305 | −0.6165 | 0.4745 | 0.7515 | 0.4965 | −0.7625 | −0.2225 |
| KIAA0703 | 0.122 | −1.114 | −0.76 | −0.04 | −0.115 | −0.097 | 0.067 | −0.375 | 0.926 | 0.413 |
| KIAA0830 | 1.6915 | 1.6605 | 0.6705 | 1.0765 | 0.9295 | 1.3885 | 0.6285 | 0.5715 | 0.0435 | −0.6375 |
| KIAA0980 | 0.1225 | 1.3045 | 0.0335 | 1.7265 | 0.5905 | 0.4965 | 0.0075 | 0.2225 | −1.0635 | −0.7805 |
| KIAA1324 | −0.171 | −0.76 | −0.104 | 0.037 | 1.306 | 0.758 | 0.717 | 0.422 | −0.815 | 0.796 |
| KIAA1363 | 1.244 | 1.222 | 0.697 | 0.138 | 1.528 | 2.525 | 1.193 | 1.798 | 0.113 | −0.056 |
| KIAA1683 | 0.2185 | −0.3645 | 0.7005 | −0.1065 | 0.4985 | −0.1705 | −0.1245 | 0.4355 | 0.5675 | 1.5295 |
| KIF12B | 0.0135 | 0.0745 | −0.5045 | 0.1535 | 0.4445 | 0.1185 | −0.0345 | 0.1955 | −0.3175 | 0.0045 |
| KIF24 | 0.2935 | −0.4255 | −0.0275 | −0.1865 | −0.5495 | −0.0605 | −0.3555 | −0.5755 | −0.1885 | 0.3525 |
| KIN | −1.8205 | −0.3285 | −0.0155 | −2.0745 | −2.0305 | −1.4505 | 2.2335 | 2.1135 | −0.0665 | −0.2095 |
| KIRREL3 | −0.6755 | −0.4365 | −0.2545 | 0.1195 | 0.1435 | −0.3455 | 0.5835 | −0.7535 | −0.2855 | −0.6365 |
| KIT | −0.4105 | −1.3205 | −1.1755 | −1.4795 | −2.9645 | −1.5085 | −0.2345 | −3.3395 | −0.3735 | −1.4805 |
| KLK5 | −1.263 | −0.161 | −0.306 | −0.442 | −0.771 | −0.638 | 1.085 | −0.62 | 0.131 | −0.872 |
| KLK6 | −0.819 | −1.153 | −0.707 | −1.006 | −1.066 | −1.149 | 0.657 | −1.912 | −0.594 | −0.986 |
| KLK7 | −0.596 | −1.094 | 0.109 | −1.733 | −0.869 | −1.395 | −0.646 | −0.295 | −0.379 | −1.491 |
| KLK8 | −0.4375 | −0.4955 | −0.3765 | −0.2015 | −0.2465 | −0.7685 | −0.6085 | −0.1075 | −0.2655 | −0.5775 |
| KNTC2 | −0.148 | 1.024 | 0.215 | −0.812 | −0.21 | −0.328 | −0.362 | −1.17 | 2.761 | −0.858 |
| KPNA2 | 0.1035 | −0.1675 | 0.0495 | −0.2695 | −0.1365 | 0.1725 | −0.0785 | 0.4625 | 0.7025 | 0.2795 |
| KREMEN2 | 2.4905 | −0.6775 | −1.5085 | −1.0375 | −0.7605 | −0.9935 | 0.6595 | −1.1395 | 3.4885 | 2.4385 |
| KRT10 | 2.7355 | −0.9475 | −2.0455 | −1.7045 | −0.3635 | −1.1705 | 0.7115 | −1.0985 | 3.8655 | 2.0635 |
| KRT13 | −1.324 | −0.063 | −0.048 | −0.902 | −1.565 | −0.592 | 0.918 | −2.316 | 0.118 | −1.246 |
| KRT15 | 0.416 | 0.129 | 0.396 | 0.794 | 0.969 | 1.147 | 1.205 | 1.418 | 2.664 | 0.914 |
| KRT17 | 3.0225 | 2.9695 | −0.8115 | −1.7845 | 0.8825 | −0.1845 | −0.2505 | −0.6285 | 1.9455 | −0.8195 |
| KRT18 | −1.998 | −0.138 | −0.43 | −1.52 | −3.265 | −1.105 | 0.047 | −2.973 | 0.925 | −2.45 |
| KRT23 | 1.9795 | −0.0295 | 0.1265 | 0.3925 | 0.0335 | 0.8095 | −0.0525 | 0.5845 | 0.2376 | −0.0275 |
| KRT6B | 0.301 | 0.914 | 0.27 | −0.816 | −0.018 | 0.334 | 0.436 | 0.668 | 1.517 | 1.894 |
| KRTAP3-3 | 0.5295 | −0.9585 | −0.5815 | −1.6305 | −0.6975 | 0.2185 | 0.2365 | −0.1945 | 0.9185 | −0.4235 |
| KRTHA7 | −0.0955 | 0.5145 | 1.3245 | −2.1455 | −0.8355 | −0.8555 | 0.3845 | −2.2655 | −0.4955 | −1.3555 |
| LAMA3 | 0.823 | 0.484 | 0.38 | 0.847 | 0.977 | 0.458 | 0.555 | 0.913 | 0.458 | 0.195 |
| LAMB3 | −0.542 | −0.078 | −0.377 | 0.278 | −0.177 | −0.421 | −9.316 | 0.29 | −0.271 | −0.378 |
| LASS2 | 0.3265 | −1.1605 | −0.9885 | −1.8105 | −0.3255 | −0.9295 | −0.3085 | −0.3295 | −1.0365 | −0.4845 |
| LAX | 9.4815 | 0.9875 | 2.0975 | −0.3465 | 1.0665 | 1.0085 | 0.4295 | 0.3035 | −0.7055 | 1.5195 |
| LBR | −0.5315 | −1.1065 | −1.2675 | −1.8115 | −1.1735 | −1.2905 | −0.5545 | −1.7215 | −1.3935 | 0.0865 |
| LCE1B | 0.1765 | −0.0385 | −0.0225 | −1.6175 | −2.0325 | 0.2765 | −0.3655 | −0.3525 | −0.1115 | 0.1025 |
| LDHB | −0.016 | −0.053 | 0.062 | 0.543 | 0.23 | 0.576 | 0.48 | 0.116 | 0.103 | 0.488 |
| LDOC1L | 1.298 | 1.058 | −0.681 | −0.187 | 1.818 | 0.993 | 0.511 | 0.673 | 1.221 | 1.494 |
| LETMD1 | −0.5615 | −1.8945 | −0.7965 | 1.2355 | −1.4735 | −1.7185 | −1.3385 | −0.8495 | −0.7875 | 0.3825 |
| LFNG | 0.622 | 0.497 | 0.88 | −0.073 | 0.233 | −0.237 | 0.140 | 0.29 | −0.102 | −0.149 |
| LGALS2 | 0.385 | 0.989 | 0.457 | 1.542 | −0.197 | 0.601 | 0.429 | 0.591 | −0.039 | 0.105 |
| LGTN | 0.493 | 0.907 | 1.338 | −0.658 | 1.276 | 0.647 | 0.484 | 0.623 | 1.374 | 1.174 |
| LNX | −0.1055 | 0.3675 | 0.3055 | −0.2105 | −1.1025 | 0.0305 | 0.0415 | 0.2115 | 1.9185 | 0.5535 |
| LOC11247 | 0.048 | −1.26 | 0.253 | 0.839 | −0.05 | −0.05 | −0.187 | 0.188 | −0.257 | 0.26 |
| LOC11492 | −0.132 | 0.072 | 0.53 | 0.4 | −0.701 | −0.134 | −0.317 | 0.258 | 0.478 | −0.151 |
| LOC11497 | −0.5035 | 0.0775 | −0.2535 | 2.0655 | 0.2345 | −0.2505 | −0.1665 | 0.0205 | −0.1445 | 0.2945 |
| LOC11621 | 0.1725 | 1.6185 | −0.2345 | −1.5355 | 1.0295 | 1.8085 | 0.3455 | 0.3965 | 2.8345 | 1.7415 |
| LOC11644 | 1.4955 | 1.4205 | 0.8325 | 1.0995 | 1.5455 | 1.4395 | 1.0435 | 1.5565 | 0.9345 | 0.2985 |
| LOC12022 | 0.2765 | 0.4115 | 0.4105 | −0.0185 | 0.3635 | 0.2725 | 0.6305 | 0.7235 | 0.9105 | 0.1355 |
| LOC12422 | −0.0645 | −0.0365 | 0.1065 | −0.4905 | 1.0765 | 0.0995 | −0.1805 | −0.3425 | 0.3075 | −0.1235 |
| LOC14654 | 0.193 | 0.643 | 0.124 | 0.463 | −0.443 | 0.242 | 0.554 | 0.6 | 1.104 | 0.059 |
| LOC15506 | 0.45 | −1.425 | −0.535 | −0.55 | −0.003 | −1.136 | −0.152 | −0.734 | 0.208 | 0.291 |
| LOC20189 | −0.6695 | −0.5815 | 2.5705 | 0.0635 | 0.2005 | −0.5265 | 0.8195 | −1.0825 | −0.5885 | 0.2255 |
| LOC25398 | −0.716 | −0.413 | −0.854 | 0.536 | −0.167 | −0.341 | −0.277 | −0.376 | −0.258 | 0.46 |
| LOC33990 | 0.567 | 0.396 | 0.419 | 0.566 | 1.074 | 1.03 | 0.37 | 0.145 | 0.476 | 0.178 |
| LOC34817 | −0.1055 | −1.0315 | 0.1755 | −0.1115 | 0.6345 | 1.0485 | 0.3195 | −0.3615 | 1.1545 | 1.3585 |
| LOC40045 | −0.14 | 0.454 | 0.467 | 0.448 | −0.124 | 0.234 | −1.243 | −1.599 | 0.042 | 1.202 |
| LOC44089 | −0.143 | −0.501 | 0.021 | −0.407 | −0.896 | −0.573 | −0.69 | −0.125 | 0.968 | 0.177 |
| LOC49386 | 1.644 | 0.78 | 0.098 | −0.607 | 0.833 | 1.106 | 0.466 | 0.572 | 1.204 | 0.91 |
| LOC51123 | −0.282 | 0.045 | −0.088 | 0.231 | −0.002 | −0.046 | 0.461 | 0.428 | −0.173 | −0.308 |
| LOC51161 | 1.9935 | 1.8465 | 1.4595 | 2.0845 | 0.1535 | 1.4275 | 1.4045 | 2.0845 | 2.2715 | 1.6315 |
| LOC51321 | −0.1275 | −0.4025 | 0.2925 | 0.0485 | −0.1266 | −0.1375 | 0.5145 | 2.0275 | −0.1175 | −9.1265 |
| LOC51760 | 0.3985 | −0.3825 | 0.0935 | 0.0025 | −0.5355 | −0.4105 | −0.5595 | −0.0405 | −0.6055 | 0.0385 |
| LOC81558 | −0.294 | −2.003 | 0.269 | 0.629 | 0.261 | 1.046 | 0.832 | 0.899 | 2.056 | 0.977 |
| LOC81569 | 0.0245 | 0.5955 | −0.4915 | −1.0595 | −0.6355 | −0.0035 | −0.0435 | 0.0315 | −0.7255 | −0.1385 |
| LOC90355 | 0.344 | 1.078 | 0.216 | 2.071 | 0.724 | −0.008 | 0.063 | 0.285 | 0.167 | 0.631 |
| LOC90701 | −1.005 | 1.514 | 0.542 | 0.408 | −1.781 | −1.829 | −1.727 | −2.039 | −1.867 | 1.067 |
| LONP | 0.3465 | 0.5815 | 0.7245 | 0.0195 | 0.1215 | 1.1885 | 0.5635 | 0.8995 | 1.3895 | 0.8365 |
| LRAP | 1.0335 | 1.7015 | 1.4455 | 0.8795 | 1.0425 | 1.1535 | 1.2765 | 1.0875 | 1.0475 | 0.7675 |
| LRBA | −0.3575 | 0.0175 | −0.0175 | 0.4125 | −0.1005 | 0.4515 | −0.0325 | 0.5745 | −0.3085 | −0.1795 |
| LRIG1 | 0.4125 | −1.5215 | −1.1535 | −1.3605 | −1.4705 | −1.9315 | −2.1315 | −2.1265 | −1.0925 | −0.4745 |
| LRP6 | −0.673 | 1.934 | 0.673 | 0.002 | 0.932 | 0.458 | 1.126 | −1.526 | 1.089 | 1.417 |
| LRP8 | −0.132 | 1.228 | 0.355 | 1.067 | −0.181 | 0.29 | 0.391 | 0.541 | 0.315 | −0.096 |
| LRRC17 | −0.4635 | −0.0015 | −0.2815 | 0.0815 | −0.2955 | −0.3815 | 0.0755 | 0.0645 | 1.3795 | 0.8415 |
| LRRC2 | 2.3875 | −0.4685 | −0.1695 | −0.1745 | 0.2895 | −0.4125 | −0.4575 | −0.8165 | −0.3205 | −0.5045 |
| LSM1 | −1.2295 | −1.1005 | −0.2895 | 0.4115 | 0.4745 | −0.5215 | −0.5645 | 1.5525 | −0.3215 | −0.5705 |
| LSM8 | −0.112 | −0.22 | −0.551 | −0.301 | −0.149 | −0.278 | −0.321 | −0.54 | 0.324 | 0.246 |
| LTB | −0.58 | −0.221 | −0.272 | −0.34 | 0.185 | 0.273 | −0.54 | 0.099 | −0.398 | 0.032 |
| LY6D | −1.146 | −0.815 | −0.025 | −1.045 | −2.325 | −1.935 | −1.145 | −0.605 | −0.275 | −1.155 |
| LYN | 0.229 | 0.065 | 0.128 | 0.331 | −0.405 | −0.033 | 0.251 | 0.127 | −0.019 | −0.131 |
| MA02L1 | 0.991 | −0.476 | −0.335 | −0.005 | −0.024 | −0.115 | 0.336 | −0.638 | 0.211 | 0.172 |
| MADP-1 | −0.0085 | −1.0885 | −0.0855 | −0.8145 | 0.1065 | −0.1385 | 0.0795 | −0.9095 | −0.2085 | −0.1145 |
| MAGEA12 | 0.93 | −0.204 | −0.191 | −0.015 | 0.072 | −0.263 | 0.208 | −0.717 | −0.39 | 0.342 |
| MAGEA2 | 0.2085 | −0.9345 | −0.0745 | −0.1935 | 0.2585 | −0.1125 | 0.1025 | −1.0965 | −0.6655 | −0.8195 |
| MAGEA3 | −1.1125 | −1.6585 | −0.6955 | −0.4625 | −0.2415 | −1.1765 | −0.6685 | −1.9195 | −0.9035 | 0.8355 |
| MAGEA4 | 0.999 | −1.136 | 0.213 | −1.169 | −0.752 | −0.131 | −0.628 | −1.525 | −1.98 | −0.042 |
| MAGED4 | −0.7495 | 1.1125 | 0.3745 | 0.7945 | 0.4825 | 0.8965 | −0.0065 | −0.3505 | 0.8405 | −0.0595 |
| MAGI1 | −0.4685 | 0.8455 | −0.4695 | −0.1045 | −1.1385 | −0.1825 | 0.1265 | 0.1715 | 1.6325 | 0.7855 |
| MAGI-3 | −0.322 | −0.217 | 0.073 | −0.324 | 0.418 | −0.03 | −0.28 | −0.156 | −0.034 | −0.367 |
| MAL2 | 0.03 | 0.817 | 0.164 | −0.436 | −0.096 | 0.375 | −1.179 | −1.844 | −0.083 | −0.143 |
| MAN2B1 | 0.7355 | −0.4795 | −0.0535 | −0.5065 | −0.6765 | −0.8315 | −0.3945 | 0.8045 | −0.8445 | 0.2435 |
| MAPK8IP2 | 1.0185 | 1.2725 | −0.7095 | −0.9435 | 0.3315 | −0.9495 | −1.2345 | −0.7235 | −1.2665 | 0.2535 |
| MARCO | −0.068 | −1.557 | 1.733 | 0.012 | 0.928 | 0.532 | −1.231 | −2.44 | 0.77 | 2.127 |
| MARLIN1 | −0.329 | −0.603 | −0.134 | −0.371 | 0.163 | −0.364 | −0.151 | −0.365 | −0.049 | −0.28 |
| MASS1 | −0.3455 | −0.3045 | −0.0355 | −0.7315 | −0.2565 | −0.2565 | −0.0405 | −0.0895 | 0.2645 | −0.5275 |
| MCM2 | 0.3735 | −0.3125 | −1.5955 | −0.7595 | 0.0005 | −0.3835 | 1.0115 | 1.2605 | 2.8176 | 1.5205 |
| MCM3 | 1.142 | 1.696 | 1.393 | 0.204 | 0.896 | 0.445 | 0.842 | 1.125 | 1.565 | 1.266 |
| MESP1 | −0.8355 | 0.6175 | 0.4165 | −0.6475 | 0.8995 | −0.5255 | 0.4345 | −1.2155 | 0.5905 | 1.0485 |
| METRN | 0.0785 | −0.2245 | −0.4265 | −0.3985 | −0.5585 | −0.4935 | −0.6405 | −0.9105 | −0.1135 | −0.1755 |
| MFAP2 | −0.004 | 0.281 | −0.046 | −0.374 | 0.243 | −0.266 | −0.246 | 0.218 | 0.191 | −0.119 |
| MFGE8 | 0.2285 | −0.1685 | −0.1785 | −0.4385 | 0.7285 | −0.1275 | 0.2275 | 0.3215 | 0.0685 | 0.1205 |
| MGAT4B | −0.505 | −0.476 | −0.294 | −0.085 | 0.352 | −0.133 | −0.134 | 0.296 | −0.557 | −0.026 |
| MGC1091 [symbol] | 0.1965 | 0.9185 | 0.8406 | 0.8295 | 1.0245 | 1.0865 | 0.6505 | 1.0875 | 0.4755 | −0.4615 |
| MGC1098 [symbol] | 0.1305 | −0.7605 | −0.2085 | −0.1275 | 0.2765 | 0.4665 | −0.4785 | −0.8335 | 0.1095 | 0.3625 |
| MGC1124 [symbol] | 0.042 | 0.041 | 0.073 | −0.815 | −0.059 | −0.05 | 0.243 | −0.27 | 0.233 | 0.166 |
| MGC1127 [symbol] | −0.114 | −0.595 | 0.147 | −0.468 | −0.058 | −0.584 | 0.077 | −0.439 | 0.017 | −0.087 |
| MGC1312 [symbol] | 0.583 | −0.189 | 0.192 | 0.956 | 0.141 | 0.63 | 0.958 | 0.785 | 0.654 | 0.256 |
| MGC1318 [symbol] | 0.441 | −0.146 | −0.187 | 0.805 | −0.417 | −0.246 | 0.116 | 0.099 | −0.614 | 0.255 |
| MGC1414 [symbol] | 0.0735 | −0.0565 | −0.1965 | 0.8455 | −0.4385 | −0.5655 | −0.0075 | 0.0225 | −0.2915 | −0.2245 |
| MGG1428 [symbol] | 1.462 | −0.304 | −0.584 | 1.437 | 0.298 | 0.852 | 1.313 | 1.364 | 0.616 | 0.238 |
| MGC1481 [symbol] | −0.34 | −0.681 | −0.031 | 0.116 | −0.045 | −0.403 | −0.64 | −0.304 | −0.471 | −0.807 |
| MGC1580 [symbol] | 0.2105 | 0.0365 | 0.1555 | 0.5555 | 0.3905 | −0.0435 | 0.3695 | 0.1115 | 0.1645 | 0.0665 |
| MGC1663 [symbol] | 0.051 | −0.452 | 0.012 | −0.218 | −0.163 | −0.382 | −0.093 | −0.496 | 0.101 | 0.008 |
| MGC1873 [symbol] | −0.451 | 0.198 | −0.009 | −0.371 | 0.871 | −0.285 | −0.264 | −1.278 | 0.83 | −0.179 |
| MGC2053 [symbol] | 0.083 | 0.647 | 0.673 | 0.987 | −1.084 | 0.738 | 1.104 | 1.515 | −0.465 | −0.161 |
| MGC2398 [symbol] | −0.11 | 0.175 | 0.123 | −0.433 | −0.588 | −0.121 | −0.209 | −0.117 | 0.129 | 0.141 |
| MGC2610 | −0.812 | 0.077 | −0.666 | −0.192 | −0.511 | −0.143 | 0.616 | 0.806 | −1.507 | −1.482 |
| MGC2714 | −0.619 | 0.416 | −0.768 | −0.128 | −1.177 | −0.681 | −0.497 | 1.089 | −0.816 | −0.814 |
| MGC2716 [symbol] | −1.0005 | 0.1855 | −0.8815 | −0.0505 | −0.9945 | −0.7725 | −0.4325 | 0.7855 | −0.7075 | −1.0055 |
| MGC2716 [symbol] | −1.6505 | 0.1015 | −1.0775 | 0.2935 | −1.3715 | −0.8885 | −0.2315 | 0.9275 | −1.0075 | −1.4285 |
| MGC2716 [symbol] | −0.936 | −0.568 | −0.971 | −0.571 | −1.044 | −1.118 | −0.716 | 0.683 | −0.882 | −0.987 |
| MGC2716 [symbol] | 0.1715 | 0.2545 | 0.6795 | −0.5735 | −0.8345 | −0.9995 | −0.3295 | 0.0985 | −0.4205 | −0.6695 |
| MGC2716 [symbol] | 1.625 | −0.416 | 0.973 | −0.333 | −0.059 | 1.337 | 1.219 | 1.459 | 0.819 | 1.187 |
| MGC2887 [symbol] | 0.357 | −0.521 | 0.469 | 0.626 | 0.37 | −0.255 | 0.616 | −0.368 | −0.132 | −0.072 |
| MGC3212 [symbol] | −1.239 | −0.412 | −0.713 | −0.45 | −0.127 | −0.315 | 0.777 | −0.03 | −1.145 | 0.123 |
| MGC3321 [symbol] | −0.3445 | −0.7305 | −1.0695 | 0.1915 | 0.5895 | −0.2775 | −0.8255 | −1.1505 | −0.9115 | 0.6765 |
| MGC3484 [symbol] | −0.1405 | 0.0815 | −0.2575 | −0.0215 | 0.4195 | −0.2445 | −0.1515 | −0.2035 | 1.3585 | 0.8725 |
| MGC3492 [symbol] | 0.271 | 0.307 | 1.356 | 0.253 | −0.116 | 1.28 | 2.516 | 1.979 | 1.246 | 0.499 |
| MGC4021 [symbol] | −0.306 | −0.259 | −0.001 | −0.388 | −0.852 | −0.685 | −0.115 | −0.062 | −0.521 | −0.591 |
| MGC4251 | 0.7245 | 0.1115 | 0.5215 | −0.3875 | 0.4705 | 0.5515 | 0.2795 | 0.6045 | 1.4355 | 0.5595 |
| MGC4308 | 0.58 | 1.254 | 1.096 | −0.289 | 1.182 | 0.881 | 1.052 | 0.736 | 0.838 | 0.737 |
| MGC4800 | −1.0255 | 3.3655 | −0.6905 | −1.0695 | −1.8135 | −1.1355 | 0.2835 | −2.0395 | −0.1815 | −1.5145 |
| MGC4659 | −0.16 | −1.184 | −0.922 | −1.099 | −1.013 | −1.035 | −0.078 | −1.317 | −1.157 | 0.224 |
| MIA | −0.0355 | −1.0835 | −0.7365 | −1.0345 | −0.8805 | −1.5855 | −0.2345 | −1.6115 | −1.4515 | 0.5375 |
| MID1 | 0.0385 | −1.7125 | −1.4285 | −2.2655 | −1.9185 | −1.8395 | −0.7105 | −1.9445 | −2.3725 | −0.4715 |
| MID1 | −0.991 | 0.126 | −0.66 | −1.218 | −0.988 | −1.187 | −0.623 | −0.129 | −0.164 | −1.504 |
| MID1 | 0.9515 | 0.6215 | 0.0195 | −0.9895 | −0.7445 | −0.9035 | −0.7055 | −0.3055 | −0.4445 | 0.7285 |
| MLF1IP | −0.9705 | 2.0925 | −0.0885 | −1.0215 | −0.1015 | −1.1945 | −1.6495 | −2.1745 | 2.5175 | −0.2945 |
| MLSTD1 | −3.825 | 1.049 | −1.481 | 1.726 | −0.912 | −1.85 | −0.449 | −4.016 | 0.161 | −2.941 |
| MMP1 | −0.005 | 0.233 | −0.146 | 0.433 | −0.446 | −0.242 | −0.188 | 0.147 | −0.231 | −0.423 |
| MMP7 | 0.0735 | −0.5435 | −0.0555 | 0.2935 | −0.3225 | 0.0415 | 0.0505 | 0.3865 | 0.1065 | −0.2105 |
| MNAT1 | −0.7175 | −0.3195 | −0.4135 | −0.4855 | −1.5375 | −0.6775 | −0.4235 | −0.3895 | 1.0075 | −0.3145 |
| MRPL14 | −0.361 | −0.175 | 0.052 | −0.523 | 0.176 | 0.07 | 0.428 | 0.054 | 0.288 | 0.424 |
| MRPL15 | 0.3265 | 0.0195 | 0.0005 | 0.3385 | −0.1895 | 0.0995 | −0.3035 | 0.2615 | 0.1845 | −0.1465 |
| MRPL27 | 0.1445 | 0.0555 | 0.0495 | 0.6365 | 0.1845 | 0.3365 | 0.5135 | 0.4555 | 0.4665 | −0.2135 |
| MRPL34 | −0.232 | 0.033 | 0.266 | −0.133 | −0.91 | −0.344 | 0.321 | 0.166 | −0.092 | −0.944 |
| MRPL43 | 0.1585 | −0.3085 | −0.0435 | 0.0345 | −0.1905 | −0.2875 | 0.1315 | −0.0615 | 0.0115 | −0.2095 |
| MRPL45 | 0.002 | 0.148 | 0.359 | −0.397 | −0.177 | −0.694 | 0.133 | 0.054 | −0.627 | −0.829 |
| MRPL48 | 0.8535 | 0.5275 | 0.6515 | 0.5565 | −0.8635 | −0.4565 | 0.4845 | 0.6905 | −0.3405 | −0.1955 |
| MRPL9 | 0.0695 | 0.0305 | −0.0325 | 0.3385 | 0.0595 | 0.1815 | −0.1765 | −0.5195 | −0.0805 | 0.0155 |
| MRPS14 | −0.197 | −0.437 | 0.076 | 0.184 | −0.52 | −0.866 | 0.654 | 1.115 | −0.654 | −0.976 |
| MRPS15 | −0.044 | −0.439 | −0.312 | −0.234 | −0.64 | −0.319 | 0.175 | 0.585 | −0.352 | −0.333 |
| MRPS17 | −0.109 | −0.293 | 0.008 | 0.039 | −0.515 | −0.684 | 0.473 | 0.028 | −0.173 | −0.776 |
| MRPS24 | 0.0475 | 0.4625 | 0.1775 | −0.4405 | −0.0015 | −0.0055 | 0.0985 | 0.2245 | 0.5965 | −0.4065 |
| MRPS25 | −0.707 | 0.003 | −0.357 | −0.26 | −0.684 | −1.676 | −0.29 | −0.801 | 0.595 | 0.172 |
| MRPS31 | −0.407 | −0.226 | 0.166 | 0.615 | −0.398 | −0.51 | −0.666 | 1.651 | −0.374 | −0.05 |
| MRPS6 | −0.2635 | 0.0425 | −0.0225 | −0.7605 | −0.7755 | −0.0695 | −0.3655 | 0.0545 | 0.1235 | −0.5685 |
| MS4A1 | 0.1435 | 0.2385 | 0.4405 | 0.7765 | 0.4795 | −0.1895 | 0.4485 | 0.5945 | −0.4705 | 0.9495 |
| MSH2 | −0.209 | −0.189 | 1.714 | −0.709 | 0.309 | 0.013 | 1.741 | 1.045 | 0.502 | −0.339 |
| MSH3 | 0.292 | 1.021 | 1.059 | 0.439 | 1.586 | 0.815 | 0.477 | 0.008 | 0.199 | 0.155 |
| MSMB | 0.015 | 0.519 | 0.428 | 0.814 | 0.156 | 0.538 | 0.328 | 0.559 | −0.013 | −0.213 |
| MTA1 | −0.3065 | 2.2975 | 0.6545 | 1.9495 | 1.0085 | 1.5955 | 0.3025 | 0.7445 | 0.3785 | −0.1045 |
| MTA3 | −0.322 | −0.799 | −0.712 | −0.332 | −0.16 | −0.254 | −0.006 | −0.153 | −0.038 | 0.182 |
| MTAC2D1 | 0.005 | 0.289 | 0.035 | −0.595 | −0.524 | −0.328 | 0.165 | 0.371 | −0.03 | −1.04 |
| MTCH1 | −0.8305 | −0.1485 | −0.0295 | −0.5465 | −0.7765 | −0.6855 | −0.4455 | −1.0835 | −0.4015 | 0.1185 |
| MTHFD1 | 1.1955 | 1.6205 | 1.2515 | 1.5265 | 1.6015 | 1.3505 | 1.2235 | 1.3295 | 0.2005 | 0.8295 |
| MTMR2 | 0.9795 | −0.8055 | −0.9695 | 1.0125 | 2.9815 | 1.2885 | 0.3865 | 0.2485 | 0.1985 | 0.6235 |
| MUC1 | 0.43 | 0.398 | −0.19 | 0.418 | 1.147 | 0.15 | 0.21 | −0.005 | 0.781 | 0.487 |
| MUC20 | −0.031 | 0.78 | 0.594 | 1.199 | −0.341 | −0.684 | 0.882 | 0.684 | 0.329 | 0.398 |
| MUM1 | 0.213 | 0.85 | 1.535 | 1.471 | 1.339 | 1.332 | 1.972 | 2.083 | 2.319 | 0.807 |
| MXI1 | −0.382 | −1.253 | 0.317 | −0.543 | −2.246 | −1.225 | −0.897 | −0.534 | 2.156 | −0.433 |
| MYB | −1.061 | −1.729 | −0.27 | −1.503 | −1.128 | −1.61 | −0.939 | −1.058 | −0.815 | −1.381 |
| MYBL1 | −0.265 | 0.402 | −1.63 | −0.1 | 0.245 | −0.059 | 0.235 | −0.057 | −0.154 | 0 |
| MYBL2 | 0.58 | 1.582 | 1.295 | 0.299 | 0.747 | 0.925 | 0.481 | 1.064 | 1.273 | 0.715 |
| MYO10 | 0.0695 | −0.0385 | −0.2235 | 0.0975 | −0.3605 | 0.1278 | 0.4185 | 0.1295 | 1.7195 | 0.5285 |
| MYO6 | 0.3025 | 1.2375 | 1.0375 | 0.6785 | −2.0505 | 0.4905 | −0.9045 | −2.1095 | 1.8465 | 0.4155 |
| MYST3 | −0.2955 | 0.1285 | 0.2765 | 0.0225 | 0.2075 | −0.0485 | 0.1955 | 0.0765 | 0.7905 | −0.1175 |
| NALP2 | −0.4035 | 0.0505 | 0.1465 | −0.0515 | 0.5465 | 0.9175 | 0.9555 | 1.2165 | 0.5315 | −0.1845 |
| NALP6 | −0.7415 | −0.3365 | −0.1985 | −0.8855 | 0.6105 | −0.1625 | −0.0005 | −0.4695 | −0.0475 | 0.9785 |
| NANS | 2.708 | 2.296 | 3.179 | 5.153 | 3.809 | 3.732 | 2.679 | 4.835 | 2.133 | 0.552 |
| NARF | 0.063 | −0.176 | 0.983 | −1.275 | 0.002 | 0.015 | 0.022 | 0.316 | −1.181 | −0.888 |
| NAT2 | 0.0005 | 0.2475 | 1.1705 | 0.5495 | 1.1945 | 0.7155 | 0.5095 | 0.4575 | 1.5845 | 0.4215 |
| NAV2 | 0.633 | −0.063 | −0.142 | −0.312 | −0.518 | 0.251 | 0.003 | 0.164 | 0.118 | −0.102 |
| NDE1 | 0.4585 | −0.0855 | −1.0705 | −0.3425 | 0.1455 | 0.6035 | 1.2885 | 0.9605 | −0.3835 | −0.2995 |
| NDP52 | 0.358 | −0.331 | 0.275 | 0.817 | −0.023 | 0.201 | −0.05 | 0.364 | 0.435 | 0.041 |
| NDRG2 | 0.67 | 0.608 | 0.169 | 0.938 | 0.373 | 0.323 | 0.42 | 1.137 | −0.23 | −0.492 |
| N0UFA7 | 0.8765 | 0.7515 | 0.2925 | 0.3515 | 0.3775 | 0.5055 | 0.7205 | 0.9515 | 0.8895 | 0.1225 |
| NDUFB1 | 0.133 | −0.07 | −0.37 | −0.112 | −0.382 | 0.083 | 0.383 | −0.302 | −0.414 | −0.191 |
| NDUFB10 | −0.027 | 0.644 | −0.287 | 0.306 | −1.321 | −0.386 | −0.352 | −0.121 | −0.599 | −0.376 |
| NDUFB2 | 0.0955 | 0.0095 | −0.2025 | −0.1755 | −0.9115 | −0.1395 | 0.4715 | 1.0445 | −0.0105 | −0.3905 |
| NDUFB5 | −0.2155 | 0.1715 | 0.5595 | −0.0755 | −0.5395 | −0.0185 | 0.4085 | 0.8115 | 0.4905 | −0.6305 |
| NDUFB6 | −0.1925 | 0.8215 | 0.2705 | 0.5635 | −0.2345 | 0.1425 | −0.1235 | 0.8215 | 0.8925 | −0.3595 |
| NDUFC2 | −0.3005 | −0.1015 | 0.4435 | −1.1975 | −1.8495 | −1.8155 | −0.9345 | −0.2055 | −0.3035 | −1.2225 |
| NEBL | −0.205 | 0.343 | 0.113 | −0.524 | 0.191 | −0.03 | 0.057 | −0.179 | −0.32 | −0.299 |
| NEK2 | 2.972 | −1.133 | 2.188 | 2.25 | 0.531 | 1.732 | 1.802 | 3.715 | 0.848 | 0.951 |
| NEK6 | 0.4535 | −0.1065 | 1.1715 | −0.1685 | 0.7925 | 0.4995 | 0.2565 | 0.4655 | −0.0885 | 1.0135 |
| NELL2 | −0.1055 | 1.6535 | 0.9995 | −1.2995 | −0.4785 | −0.4885 | −0.0735 | −0.1145 | 1.4425 | −0.0015 |
| NEURL | 0.801 | 1.449 | 0.869 | −0.395 | 0.382 | 1.438 | 1.462 | 0.896 | 1.048 | 0.185 |
| NFE2L3 | −0.8676 | 0.1825 | 0.4615 | −0.5115 | −1.8285 | −1.4075 | −0.4255 | −1.5935 | −1.4895 | −0.0685 |
| NFIA | 1.261 | −0.022 | −0.248 | 1.092 | 0.964 | 1.149 | 1.038 | 1.327 | 0.69 | −0.497 |
| NFIB | −0.5695 | −0.0485 | 0.5435 | 0.1225 | −1.1695 | −0.0345 | 0.4115 | 0.5975 | 0.8585 | −0.0275 |
| NINJ1 | 0.7045 | 0.0215 | −0.3685 | 0.5615 | −0.0295 | −0.2115 | −0.2045 | 0.0855 | −0.1175 | 0.1585 |
| NIPSNAP [symbol] | −0.3515 | −0.0515 | 0.3625 | −1.1945 | 0.1355 | −0.2555 | 0.4745 | 0.0905 | −0.2425 | −0.2165 |
| NKX8-2 | −0.3785 | −0.2335 | 0.4685 | −1.5985 | −0.2415 | −0.3675 | 0.5495 | 0.0105 | −0.2345 | −0.1825 |
| NME1 | 0.89 | 0.615 | 0.87 | 1.499 | 1.605 | 0.925 | 0.866 | 0.712 | 0.96 | 0.261 |
| NME2 | −1.7395 | −1.9955 | 0.7395 | 4.6925 | 0.4845 | 1.5325 | 2.0315 | 2.3665 | 0.0095 | −0.6075 |
| NME3 | −0.6095 | −0.1235 | 0.1685 | −0.1095 | −1.1095 | −1.0275 | −0.3685 | −0.1345 | −0.6035 | −0.9925 |
| NMES1 | 0.26 | 0.044 | −0.548 | 0.033 | −0.822 | −0.644 | −0.68 | −0.654 | −0.408 | 0.11 |
| NMI | −0.2475 | −0.1685 | 0.1735 | −0.9405 | −0.5875 | −0.3545 | −0.0285 | −0.3115 | 0.2125 | −0.7245 |
| NOC4 | −0.5265 | −0.0915 | −0.5415 | 0.0895 | −0.1885 | −0.2735 | −0.1115 | 0.2435 | −0.4905 | −0.9975 |
| N0LC1 | −0.5185 | 0.8985 | 0.4205 | −2.4805 | −1.1545 | −0.4945 | 0.0165 | 0.0205 | −0.0685 | 0.1225 |
| NotFound | 0.1555 | −0.1425 | 0.1395 | −0.7385 | −0.3205 | 0.3005 | −0.3075 | −0.2635 | 0.8585 | 0.3535 |
| NotFound | −0.554 | −0.871 | −0.651 | −0.154 | −0.628 | −0.661 | −0.152 | −0.221 | −0.674 | −0.791 |
| NotFound | 0.193 | −0.1 | 0.592 | 2.234 | 0.663 | 0.739 | 0.023 | 0.602 | −0.35 | −0.512 |
| NotFound | −0.144 | −0.312 | −0.056 | 1.26 | −0.306 | −0.172 | 0.238 | 1.984 | −0.126 | −0.31 |
| NotFound | −0.5165 | 0.2885 | −0.4565 | −0.3215 | −0.1375 | 0.1265 | −1.7885 | 0.2605 | −0.5795 | −0.8055 |
| NotFound | −0.687 | 1.108 | 2.818 | −0.155 | −1.152 | −0.632 | 0.167 | −1.151 | 1.769 | −0.586 |
| NotFound | 0.1085 | 2.3165 | −1.8825 | −0.9235 | −1.4085 | −1.5885 | −1.5005 | −1.1995 | −1.4495 | 0.1915 |
| NotFound | −0.514 | −0.982 | −1.225 | 0.283 | −0.411 | −0.602 | −0.808 | −1.187 | −0.505 | 0.427 |
| NotFound | −0.1655 | 0.0895 | −0.3145 | −1.5395 | 0.2355 | −0.1585 | −0.3635 | −0.1935 | −0.0905 | 0.0775 |
| NotFound | −2.2745 | −0.1925 | −1.7735 | 0.9065 | −1.9325 | −2.1515 | −0.2675 | 0.3805 | −1.8825 | −2.6605 |
| NotFound | 0.2355 | −0.5385 | 0.0765 | 0.3345 | 1.0245 | 0.1585 | 0.6185 | 0.4825 | 0.3065 | 0.3235 |
| NotFound | −0.4765 | −0.0085 | −0.3055 | 0.3645 | 0.2105 | 0.0245 | −0.1465 | −0.0795 | −0.1865 | 0.0225 |
| NotFound | 0.801 | 0.118 | −0.045 | 0.361 | 0.781 | 0.207 | 0.407 | −0.042 | 0.074 | 0.016 |
| NotFound | 0.145 | 0.052 | 0.071 | 0.693 | 0.87 | 0.29 | 0.134 | 0.25 | 0.396 | 0.427 |
| NotFound | 0.621 | 0.28 | 0.372 | 0.851 | 0.746 | 1.032 | 1.107 | 1.41 | 2.629 | 1.074 |
| NotFound | 1.0945 | −0.0915 | −0.2185 | 0.3165 | 1.1955 | 1.1025 | 0.5445 | 0.3215 | 1.4925 | 1.5435 |
| NotFound | −0.544 | −1.957 | −0.3 | 1.712 | 0.975 | −0.521 | −0.31 | −0.704 | 1.67 | 0.849 |
| NotFound | 0.364 | −0.152 | −0.055 | −0.412 | −0.734 | −0.503 | 0.105 | −0.52 | −1.12 | −0.652 |
| NotFound | 0.4215 | 0.1015 | −0.1225 | 0.7085 | 0.3045 | 0.4685 | −0.4585 | −0.6885 | 0.1205 | −0.2085 |
| NotFound | 1.3095 | −2.2375 | 0.4345 | 3.0825 | −1.9635 | 0.6455 | 1.0635 | 0.1745 | −2.1895 | −0.9735 |
| NotFound | 0.657 | 0.078 | 0.066 | 0.98 | 0.465 | 0.387 | 0.511 | −0.348 | −0.101 | 0.783 |
| NotFound | −0.0775 | −0.5285 | −0.6935 | −0.8155 | −0.5175 | −0.7265 | −0.2165 | −0.8255 | −0.7195 | −0.2165 |
| NotFound | 3.353 | 2.514 | 3.877 | 4.724 | 3.997 | 4.193 | 3.799 | 4.527 | 2.254 | 0.726 |
| NotFound | 0.184 | 1.232 | −0.605 | 0.026 | 0.428 | −0.296 | −0.349 | −0.234 | −0.164 | 0.582 |
| NotFound | −0.8405 | −1.0105 | −1.2895 | −1.9265 | −2.7225 | −2.3305 | −0.3825 | −0.1605 | −0.6735 | 0.2735 |
| NotFound | 0.256 | 1.071 | 1.08 | −0.778 | −0.139 | −0.534 | 0.186 | −0.157 | −0.507 | −1.027 |
| NotFound | 0.2685 | 1.3145 | −0.6615 | 0.6425 | 0.1945 | −0.5105 | −0.5015 | −0.3235 | −0.5515 | 0.1365 |
| NotFound | 0.708 | −0.647 | −0.936 | −0.529 | −0.867 | −1.078 | −0.576 | −1.487 | −0.585 | 0.357 |
| NotFound | 0.063 | 0.405 | 0.226 | 0.9 | −0.938 | 0.397 | 0.096 | 0.529 | −0.045 | −0.45 |
| NotFound | −0.5565 | −0.3725 | −0.7285 | −0.3375 | −0.3435 | −0.4585 | −0.2345 | 0.4915 | −0.0415 | −0.2625 |
| NotFound | 1.0205 | 0.6785 | 0.2185 | 0.0725 | −0.7785 | −0.9155 | −0.8675 | −0.3715 | 0.3585 | 0.9805 |
| NotFound | 0.4415 | 0.9405 | −1.0265 | −0.0715 | 0.2475 | −0.2065 | −0.3615 | 0.2155 | −0.1905 | 1.4765 |
| NotFound | −0.0065 | 1.8915 | −0.8855 | −0.5695 | −0.5105 | −0.8035 | −0.9815 | −0.6215 | −0.8495 | −0.0225 |
| NotFound | 0.7355 | 0.0855 | 0.8305 | 1.4175 | 0.3565 | 1.2185 | 0.8455 | 1.4595 | 0.0845 | −0.6595 |
| NotFound | 0.508 | 1.069 | 0.667 | −0.023 | −0.034 | −0.285 | −0.289 | −0.245 | −0.062 | 0.425 |
| NotFound | 0.6095 | 1.6015 | −0.1775 | −0.5965 | −0.4625 | −0.1935 | 0.2965 | 0.8775 | 0.0435 | 0.5725 |
| NotFound | −0.4725 | −0.7625 | −0.3825 | −0.2865 | −0.3775 | −0.7605 | −0.8375 | −1.2205 | 0.1115 | 0.0325 |
| NotFound | 1.5355 | 0.7765 | −0.6285 | 0.1005 | 0.4775 | −0.5935 | −0.3185 | −0.1925 | 0.5745 | 0.5615 |
| NotFound | 0.2215 | −0.2715 | −0.6425 | 0.2985 | 0.5945 | 0.4785 | 0.0115 | 0.1875 | −1.2625 | −0.0715 |
| NotFound | −0.026 | −0.993 | −0.659 | −0.738 | 0.637 | −1.129 | −0.71 | −1.087 | 0.281 | 0.328 |
| NotFound | 0.344 | 0.256 | −0.008 | 0.442 | −0.208 | 0.125 | −0.027 | −0.236 | 0.139 | −0.48 |
| NotFound | −0.097 | −0.172 | −0.445 | 0.026 | −0.289 | −0.481 | 0.02 | 0.939 | −0.638 | −0.831 |
| NotFound | 0.4465 | 1.3585 | −0.6505 | 0.4105 | 0.0865 | −0.5495 | −0.5415 | −0.2825 | −0.5295 | −0.0145 |
| NotFound | −0.5225 | 0.6735 | 0.4045 | −0.0045 | −0.6585 | 0.0775 | −1.1585 | −0.6035 | 0.5095 | 0.0845 |
| NotFound | 0.397 | −0.349 | −0.041 | 0.171 | 0.22 | 0.067 | −0.047 | −0.088 | −0.192 | −0.094 |
| NotFound | −0.0265 | −0.2755 | 0.1535 | 0.5115 | 0.5015 | −0.0055 | −0.1335 | 0.0565 | 0.5645 | −0.1485 |
| NotFound | −0.0355 | −1.4905 | 0.8155 | 1.7275 | 1.0905 | 0.3365 | −0.6275 | −1.4945 | 0.6585 | 0.1365 |
| NotFound | 0.6845 | 0.2565 | 0.3435 | −0.0355 | 0.9555 | 0.2805 | −0.0835 | −1.6345 | 0.8995 | 1.0765 |
| NotFound | 0.2815 | 0.6435 | −0.0085 | 1.3885 | −0.3355 | −0.5955 | −0.1115 | −0.0615 | −0.2315 | 0.2335 |
| NotFound | 0.371 | 0.441 | 0.091 | −1.135 | −0.541 | 0.356 | −0.635 | 0.38 | 0.146 | 0.414 |
| NotFound | 0.647 | 0.773 | 1.135 | 0.971 | 1.639 | −0.202 | 0.8 | 0.631 | 0.825 | −0.483 |
| NotFound | 0.1235 | −0.0345 | 0.5245 | 2.0615 | 0.6835 | 0.8285 | 0.3105 | 0.7095 | −0.1385 | −0.1145 |
| NotFound | 0.9475 | 0.3015 | 0.0405 | 0.3835 | −0.2225 | 0.4435 | 1.1205 | 1.3835 | 0.4876 | 0.5345 |
| NotFound | 0.7955 | −0.2855 | −0.0405 | 2.0815 | 1.2185 | 1.6255 | 0.8935 | 1.3455 | 1.4135 | 0.4385 |
| NotFound | −0.2845 | 1.7595 | −0.3655 | 0.4095 | −0.8165 | −0.2425 | −0.8175 | −0.3025 | −0.5015 | −0.1875 |
| NotFound | −1.488 | −0.154 | 1.999 | 0.389 | 1.658 | 2.171 | 0.246 | −0.203 | 1.002 | −1.141 |
| NotFound | 3.338 | 1.171 | 1.32 | −0.418 | 3.598 | −0.064 | 1.012 | −0.08 | 0.696 | 3.755 |
| NotFound | −0.3315 | 0.0685 | −0.1275 | −0.2355 | −0.2115 | −0.0755 | −0.1895 | 0.2475 | −0.2185 | −0.8055 |
| NotFound | 0.896 | −1.027 | −1.431 | 0.418 | −1.273 | −0.54 | −0.051 | −1.149 | −0.364 | 0.495 |
| NotFound | −0.347 | −0.447 | −0.445 | −0.103 | −0.553 | 0.254 | −0.446 | −0.888 | 0.059 | 0.355 |
| NotFound | 1.1025 | 0.2255 | 0.5055 | −1.1445 | 0.7025 | 0.8115 | 0.8375 | 0.2935 | 1.1615 | 0.1905 |
| NotFound | 0.476 | 0.279 | −0.179 | −0.583 | 0.279 | 0.021 | −0.18 | 0.119 | −0.012 | 0.045 |
| NotFound | 0.548 | 1.228 | −1.31 | −0.216 | 0.067 | −0.307 | −0.445 | 0.328 | −0.166 | 1.695 |
| NotFound | −1.0085 | −1.4325 | −0.7385 | 1.2205 | −0.7435 | 0.0785 | −1.0375 | −1.3915 | −0.6895 | −0.0345 |
| NotFound | 0.883 | 0.292 | 0.605 | 0.801 | 0.631 | 1.834 | 0.289 | 0.175 | 0.106 | 1.376 |
| NotFound | −0.8885 | −0.6285 | −0.0695 | −1.0385 | −0.0225 | −0.6145 | −0.3716 | −0.4905 | 0.5285 | −0.1035 |
| NotFound | −2.2945 | −0.4585 | −0.0585 | −0.0805 | 0.0085 | −0.0125 | −0.0755 | −0.4405 | −0.5025 | −0.2675 |
| NotFound | 0.438 | 1.305 | −0.634 | 0.124 | 0.251 | −0.491 | −0.463 | −0.342 | −0.511 | 0.208 |
| NotFound | 0.0295 | −0.0845 | −0.0085 | −0.7415 | −0.2795 | −0.6685 | −0.0025 | −0.0805 | −0.5885 | −0.9005 |
| NotFound | 0.6 | 0.522 | 0.19 | −0.48 | 1.488 | 0.83 | 1.02 | 0.701 | 0.543 | 0.324 |
| NotFound | 0.578 | 0.383 | 0.931 | −0.029 | 0.134 | 0.157 | 0.151 | 0.948 | 0.491 | −0.515 |
| NPDC1 | 0.1765 | −0.3035 | −0.0125 | −0.1185 | −0.5285 | −0.6775 | −0.0665 | −0.4225 | −0.1435 | 0.3975 |
| NPEPPS | 0.538 | 0.217 | 0.294 | 0.844 | −0.207 | 0.55 | 0.611 | 0.639 | 0.698 | 0.375 |
| NPY | −0.336 | 0.123 | −0.182 | 0.111 | −0.906 | −0.896 | −1.059 | −0.999 | 0.024 | −0.428 |
| NQO3A2 | 0.985 | 0.881 | 1.08 | −0.09 | −0.11 | 1.703 | −0.152 | −0.712 | 1.331 | 1.845 |
| NRAS | 0.737 | 2.554 | 1.14 | −0.219 | 1.53 | 2.583 | 2.074 | 2.243 | −0.117 | 0.733 |
| NRIP1 | 2.0225 | 1.1525 | 0.4635 | 1.0945 | 1.7335 | 1.6965 | 0.4655 | 1.2805 | −0.1845 | 1.5425 |
| NTN4 | 0.2055 | −0.2875 | −0.3035 | 0.3725 | −0.4825 | −0.1385 | −0.2705 | 0.1745 | −0.1405 | −1.0205 |
| NUCB2 | 0.7245 | 0.7305 | 0.4835 | −0.0815 | −0.0745 | 0.6155 | 0.4235 | 0.8785 | 0.5655 | −0.2375 |
| NUDT5 | 0.248 | −0.411 | 0.26 | 0.209 | −0.901 | −0.096 | −0.073 | 0.065 | 0.501 | −0.327 |
| NUDT6 | 0.19 | −0.008 | 0.043 | −0.223 | 0.938 | 0.457 | 0.385 | 0.461 | −0.256 | −0.222 |
| OCIAD2 | −0.3055 | 0.6275 | −0.0075 | −0.0535 | −0.1795 | 0.1895 | 0.2325 | −0.0215 | 0.5985 | −0.1495 |
| OFD1 | 0.1715 | −0.0475 | −0.0775 | −0.9315 | −0.1495 | 0.0925 | 0.6465 | 0.5735 | 1.7865 | 0.7275 |
| OGFRL1 | −0.087 | −0.336 | −0.213 | 0.312 | 0.129 | −0.061 | 0.443 | 0.173 | 0.637 | 0.816 |
| OPLAH | −0.3325 | 0.2155 | −0.0285 | −0.7645 | 1.1445 | −0.0575 | −0.4855 | −1.0995 | −0.9565 | 1.0335 |
| ORMDL3 | 0.593 | −0.358 | −1.083 | −0.036 | 0.116 | −1.682 | −0.78 | −2.251 | −1.116 | 1.511 |
| OSBPL3 | −0.23 | −0.173 | −0.135 | −0.695 | −0.417 | −0.143 | −0.141 | 0.054 | 0.211 | 0.299 |
| OSR2 | 0.1516 | −0.2425 | −0.5345 | −0.0655 | −0.8475 | −0.7315 | −0.3485 | −0.1955 | 0.0915 | 0.1835 |
| OSTF1 | −0.952 | 0.593 | 0.737 | 0.492 | 1.441 | 0.656 | 1.217 | 1.526 | 1.288 | 0.432 |
| P2RX1 | −0.4255 | −0.8335 | −0.8545 | 0.0035 | −0.1125 | 0.1825 | 0.4475 | −0.0845 | −1.0445 | −0.9445 |
| P2RY2 | −0.68 | 0.483 | −0.016 | −0.593 | −0.248 | 0.322 | 0.602 | 0.501 | −0.457 | −0.287 |
| PACAP | −0.493 | −0.449 | −0.197 | 0.339 | 0.695 | −0.396 | −0.297 | −0.724 | −0.64 | 0.923 |
| PADI2 | 0.0435 | 2.0105 | −0.7235 | −1.9055 | −0.7485 | −0.3645 | −0.1795 | −0.9685 | 0.0025 | 0.4816 |
| PAPD5 | 0.313 | 0.287 | 0.009 | 0.15 | −0.292 | −0.28 | −0.402 | −0.463 | −0.182 | −0.812 |
| PAPSS2 | 0.973 | 0.051 | 0.446 | −0.91 | −0.335 | −0.714 | −0.231 | −0.208 | −0.483 | −0.738 |
| PARD8A | −0.995 | 1.214 | 0.241 | 1.177 | −0.161 | 0.123 | 0.147 | 0.679 | 0.183 | 0.197 |
| PARP1 | 0.231 | −0.117 | 0.148 | 0.74 | 0.187 | 0.721 | −0.157 | −0.128 | −0.028 | 0 |
| PARP9 | 0.7816 | 0.0195 | 0.9995 | −1.2475 | 1.8235 | 1.5445 | −0.5465 | −0.9985 | 2.1055 | 2.0495 |
| PAXIP1L | −0.2295 | −0.6655 | −1.2755 | 1.2115 | 0.6005 | −0.8555 | 0.0885 | 0.5205 | −0.7165 | −0.0725 |
| PBX3 | −0.7715 | −0.2275 | −0.3265 | −0.0245 | 0.2935 | −0.0205 | −0.2215 | 0.1635 | −0.0645 | 0.7815 |
| PCDHB11 | 0.565 | −1.261 | −1.372 | 0.173 | 1.459 | −1.582 | 1.075 | 1.589 | −1.434 | −1.685 |
| PCDHB16 | 1.062 | 0.928 | 0.914 | 1.99 | 1.058 | 1.733 | 0.485 | 0.987 | −0.17 | −0.023 |
| PCDHB2 | −0.7725 | −0.0325 | −0.0725 | −0.8145 | −0.9515 | −0.5405 | −0.3615 | −0.0635 | 0.2225 | −0.3565 |
| PCM1 | −0.2105 | −0.4585 | −0.2725 | −0.1385 | 0.0125 | −0.1375 | −0.1485 | 0.1805 | −0.3245 | −0.0825 |
| PCNA | −0.402 | 0.582 | −0.399 | 0.42 | −1.491 | −0.433 | −0.469 | −0.056 | 0.326 | −0.334 |
| PDC01 | 0.3305 | 0.6845 | 2.6435 | 1.5125 | 1.0945 | 1.6505 | 1.9335 | 1.9975 | 0.8655 | 1.1835 |
| PDC010 | −0.679 | −0.41 | −0.118 | −0.245 | −0.807 | −0.833 | −0.525 | −0.13 | −0.317 | −1.199 |
| PDCD4 | 0.2585 | −0.0095 | 0.2605 | 0.0775 | −0.4225 | −0.2065 | 0.1075 | 0.2175 | 0.5315 | −0.4025 |
| PDCD5 | −0.1655 | 0.0775 | 0.0815 | −1.0125 | 1.2455 | −0.0725 | 0.1485 | −1.0015 | −0.1895 | 2.1315 |
| PDCD8 | −0.2755 | −0.4825 | −0.4285 | −0.7715 | −0.3465 | −0.5765 | −0.2025 | −0.1175 | 0.0375 | −0.1715 |
| PDE4DIP | 0.2635 | 1.2755 | 0.3655 | 0.8265 | −0.7915 | 0.5825 | 0.6595 | 0.5765 | −0.1475 | −0.6276 |
| PDHA1 | −0.77 | −2.739 | 2.224 | −0.885 | −2.097 | −2.31 | −0.935 | −2.548 | 1.997 | −0.692 |
| PECI | 0.4285 | −0.1715 | 0.2205 | 0.0665 | −1.1025 | 0.1685 | −0.2115 | 0.4525 | −0.2395 | −0.1545 |
| PEG10 | −1.3465 | 0.2025 | −0.4015 | 0.2725 | −0.9725 | −0.8245 | 0.0965 | −0.4625 | 0.1755 | −0.3735 |
| PER2 | 0.02 | 0.523 | 0.267 | 0.192 | 0.417 | 0.131 | −0.028 | −0.205 | −0.117 | −0.166 |
| PERP | 0.2205 | 0.6775 | 0.4495 | −0.4695 | 0.6275 | 0.4545 | 0.0215 | −0.0595 | 0.1645 | 0.1205 |
| PEX1 | −0.012 | 0.072 | 0.165 | 0.022 | −0.464 | −0.707 | −0.195 | −0.193 | −0.812 | −0.849 |
| PFAAP5 | 0.4015 | 0.9525 | 1.8915 | −0.1995 | 0.3905 | 0.6016 | 0.4625 | 0.3145 | −1.1275 | 0.6295 |
| PFDN2 | −0.304 | 0.16 | 0.083 | 0.112 | −0.437 | 0.283 | 0.279 | 0.263 | 0.734 | 0.08 |
| PGM1 | −0.1095 | −0.0045 | −0.0785 | 0.0045 | −0.6815 | −0.1865 | −0.1455 | −0.0575 | 0.1705 | 0.5385 |
| PGR1 | −0.3095 | −0.5595 | −0.9765 | −0.9995 | −0.4135 | −0.7435 | −0.7215 | −1.6085 | −1.1205 | −0.5816 |
| PHAX | 0.45 | −0.181 | 0.028 | −0.074 | −1.295 | −0.472 | 0.327 | −0.082 | −0.627 | −0.034 |
| PHGDH | 0.8025 | 0.6775 | 0.7275 | 0.7785 | 0.0965 | 1.1435 | 0.3925 | 1.0835 | −0.3375 | 0.4955 |
| PHYH | 0.8655 | 0.4455 | 0.3355 | 0.0215 | 0.5695 | 0.9785 | 0.8365 | 1.0745 | 0.1155 | 0.7335 |
| PI85PA | 0.0745 | −0.3845 | −1.0115 | 0.5235 | −0.7475 | −0.5925 | −0.1635 | −0.7145 | −0.3395 | −0.0995 |
| PIGT | 0.166 | 0.271 | −0.188 | 0.271 | −0.096 | 0.799 | 0.055 | −0.419 | 0.077 | −0.001 |
| PIM2 | −0.4495 | −0.4565 | −0.5235 | −0.7195 | −0.4425 | −0.3795 | −0.1436 | −0.6655 | −0.6585 | 0.4245 |
| PISD | 2.838 | −1.08 | 2.231 | 1.558 | −0.707 | 2.655 | 1.783 | 2.187 | 1.154 | −1.152 |
| PITPNC1 | −0.3145 | −0.4315 | 0.3865 | −0.9255 | −0.6375 | −1.0285 | −0.2885 | −0.4865 | 0.6305 | −1.0035 |
| PKIB | 0.53 | 0.214 | −0.253 | 1.307 | 0.87 | 1.194 | 0.419 | −0.278 | 0.939 | 1.054 |
| PKMYT1 | −0.888 | −1.008 | −0.898 | 0.97 | −1.282 | −0.623 | −1.041 | 0.384 | −1.839 | −1.084 |
| PLA2G4B | −0.4535 | −0.4245 | −0.5995 | −0.6995 | −0.1515 | 0.4055 | 0.0355 | −1.0845 | 0.1095 | 0.2255 |
| PLAC8 | 0.12 | −0.788 | −0.444 | 0.111 | 0.324 | −0.529 | 0 | −0.021 | −0.364 | −0.132 |
| PLCB1 | 0.7205 | 0.3785 | −0.1175 | −1.1555 | 0.6525 | 0.8125 | −0.0075 | 0.4665 | 0.4985 | −0.3685 |
| PLCXD1 | −0.071 | −0.487 | −0.358 | −0.817 | 0.895 | −0.295 | −0.006 | −0.848 | 0.156 | 0.883 |
| PLEK2 | 0.3505 | 0.2625 | −0.0685 | −0.9145 | −0.1835 | −0.2645 | −0.2685 | −0.0915 | −0.4975 | 0.0975 |
| PLO0 | −0.2925 | −0.3195 | 0.2505 | 0.0095 | 0.2455 | 0.4605 | −0.1995 | −0.6845 | −0.3075 | −0.2765 |
| PNAS-4 | 0.3525 | −0.0755 | −0.2835 | −1.1155 | −0.6055 | −0.5615 | −0.1375 | −0.8325 | −1.2165 | 0.3195 |
| PNLIPRP2 | 0.1005 | −0.2965 | −0.4225 | 0.1075 | −0.3605 | 0.3805 | 0.4655 | 0.9475 | 0.8675 | 0.3725 |
| PNMA3 | −0.0705 | 0.0575 | −0.0465 | −0.0975 | −0.5295 | −0.5545 | −0.4605 | −0.7745 | −0.6155 | −0.4815 |
| POLB | −0.229 | 0.112 | 0.097 | 0.835 | 0.46 | 0.078 | 0.183 | 0.038 | −0.312 | −0.507 |
| POLR2F | 0.1445 | −0.1615 | −0.4615 | 0.0735 | 0.2785 | 0.0675 | −0.0975 | −0.3325 | 0.9165 | −0.1435 |
| POLR2H | −0.3145 | 0.2345 | −0.2575 | 0.2675 | −0.5165 | −0.3235 | −0.0645 | 0.3085 | −0.0285 | 0.2455 |
| POMC | −0.189 | −0.582 | −0.301 | −0.064 | −0.412 | −0.431 | −0.17 | −0.381 | 0.139 | −0.773 |
| POP4 | −0.497 | 0.423 | 0.107 | −0.063 | 0.818 | 0.432 | 0.256 | −0.604 | 0.563 | 0.731 |
| POP7 | −0.806 | −0.191 | 0 | 0.148 | 0.128 | 0.096 | −0.381 | −0.312 | 0.023 | −0.315 |
| PP1057 | −0.0525 | 0.9525 | 0.2235 | 0.7945 | 0.8185 | 0.0925 | 0.3305 | 0.8125 | −0.7635 | −0.5505 |
| PPFIA1 | −0.089 | −0.423 | −0.798 | −0.254 | 0.189 | −0.671 | −0.277 | −0.594 | −0.337 | 0.438 |
| PPFIBP2 | −0.4185 | 0.1185 | 0.3765 | 0.2235 | 0.3185 | 0.0145 | 0.1925 | 0.5315 | −0.1375 | 0.3895 |
| PPGB | 0.0315 | −1.0305 | 0.4085 | 0.4065 | −0.0525 | 1.5195 | 1.0585 | 1.2485 | 0.8605 | −1.0675 |
| PPHLN1 | −0.4035 | −0.4025 | −0.4085 | 0.0305 | −1.0545 | −0.2585 | −0.0475 | −0.2835 | −0.5585 | −0.5705 |
| PPIF | 0.1835 | −0.0175 | 0.5935 | 0.1995 | 1.2085 | 0.0725 | 0.6705 | 0.4835 | 0.0415 | −0.0975 |
| PPIL1 | −0.3005 | −0.2585 | 0.3655 | −1.0935 | 0.8735 | 0.1955 | 0.0065 | 0.1745 | 1.2915 | −0.1875 |
| PPOX | 0.897 | −0.609 | 1.289 | 3.639 | 0.447 | 0.745 | 2.39 | 3.126 | 0.224 | 0.294 |
| PPP1R16 [symbol] | −0.17 | 0.043 | 0.177 | 0.719 | 0.836 | 0.34 | 0.043 | 0.201 | 0.397 | 0.077 |
| PPP1R3C | 0.801 | 0.853 | 0.606 | −0.691 | 0.228 | 0.421 | 0.428 | 0.523 | −0.161 | −0.179 |
| PPP2R2D | 0.8205 | −0.9625 | −0.9155 | −0.3995 | −0.7035 | −1.0575 | −0.0075 | −1.2085 | −0.2775 | −0.6585 |
| PPP2R5A | −0.599 | −0.819 | 0.111 | −1.027 | −0.491 | −0.873 | −0.764 | −0.945 | −0.294 | −0.711 |
| PRAME | −0.1175 | 0.3155 | 0.2985 | 0.4365 | −0.3985 | −0.7715 | −0.0725 | 0.0015 | 0.0745 | −0.5525 |
| PRC1 | −0.231 | −0.261 | −0.457 | 2.811 | 2.016 | −0.091 | −0.361 | −0.542 | −0.094 | 0.046 |
| PRIM2A | −0.0985 | −0.4565 | −0.6055 | −1.0275 | −0.9995 | −0.7745 | −1.2315 | −0.2385 | −1.0425 | −0.7015 |
| PRKACB | −0.528 | 0.125 | 0.122 | 0.321 | −0.012 | 0.085 | 0.44 | 0.596 | −0.715 | −0.318 |
| PRKCB1 | −0.708 | 0.068 | −0.102 | −0.57 | −0.64 | −0.385 | −0.126 | −0.111 | −0.082 | −0.259 |
| PRKCI | 0.464 | 0.312 | 0.377 | −0.535 | 1.08 | 0.27 | 0.282 | −0.059 | 0.43 | 1.049 |
| PRKRIR | −0.9245 | −1.0875 | −0.4745 | −0.7965 | −0.7345 | −1.1235 | −0.2505 | −0.4095 | −0.8275 | 0.3055 |
| PRKWNK1 | −1.2885 | −0.7895 | −1.4575 | −1.8755 | −2.1135 | −1.8765 | −0.8515 | −2.2525 | −1.0105 | −0.4855 |
| PRKX | −0.199 | −0.285 | −0.213 | 0.496 | −0.059 | −0.178 | −0.188 | 0.217 | −0.297 | −0.607 |
| PRNP | −0.6025 | 0.3515 | 0.0325 | −0.5145 | −1.2175 | −0.7435 | −0.2375 | −0.4605 | −0.1545 | −0.2225 |
| PROL5 | −0.2165 | −0.1585 | −0.1475 | −0.2635 | −0.0475 | 0.2135 | −0.2035 | 0.2745 | 0.1075 | 0.0155 |
| PRPF18 | −0.8765 | −1.3275 | −0.4295 | −0.9385 | −1.3095 | −0.4745 | −0.1155 | −1.2795 | −0.9515 | 0.6545 |
| PRPSAP1 | 0.9485 | 0.4245 | 0.4505 | −0.4935 | −1.2695 | 0.2985 | 0.2935 | −1.1635 | −0.5325 | −0.7355 |
| PRSS12 | 2.2545 | 1.1295 | 2.6475 | 4.3285 | 4.5715 | 3.7915 | 2.3775 | 3.2255 | −0.5315 | −0.0615 |
| PRSS16 | −0.3945 | 0.4516 | −0.3255 | −0.1915 | −0.5745 | −0.2825 | −0.5975 | 0.0035 | −0.2345 | −0.4016 |
| PSD3 | −0.3805 | −0.6915 | −0.8555 | −1.0125 | −0.3716 | −0.7195 | −0.2175 | −0.3605 | −0.2835 | −0.4745 |
| PSMA3 | −0.082 | 0.055 | −0.054 | −0.245 | 0.557 | −0.004 | 0.24 | 0.083 | 0.078 | −0.177 |
| PSMA7 | −0.452 | 0.658 | 0.007 | −0.608 | −1.033 | −0.218 | −0.288 | 0.082 | 0.14 | −0.679 |
| PSMC5 | 0.3055 | −0.3725 | −0.3615 | −1.0355 | −0.5995 | −0.2435 | −0.8825 | −0.0275 | 0.1385 | 0.0525 |
| PSMC6 | −0.282 | −0.097 | −0.167 | −0.725 | −0.475 | −0.234 | −0.392 | −0.086 | −0.396 | −0.892 |
| PSMD12 | 0.1935 | −0.5805 | −0.3875 | 0.5015 | −0.2315 | −0.1225 | 0.0555 | −0.1415 | 0.3855 | 0.1416 |
| PSMD12 | −0.9535 | −1.7605 | −0.7925 | 0.7535 | −2.2145 | −2.1605 | −1.4225 | −0.4985 | −1.1705 | 0.5895 |
| PSMD3 | 0.1805 | 0.2555 | 0.0165 | −0.8545 | 0.6445 | 0.6285 | 0.7275 | 0.4825 | 0.2705 | 0.1585 |
| PSPH | 0.278 | 0.541 | 0.833 | −0.528 | −0.378 | −0.26 | 0.372 | −0.099 | 0.956 | 1.179 |
| PTD012 | 0.888 | −0.452 | 3.03 | −0.559 | −0.604 | −1.062 | 0.445 | −0.438 | −0.228 | 0.537 |
| PTGER3 | 0.596 | −0.702 | −0.076 | 1.424 | 1.427 | 1.181 | 0.712 | 1.196 | 0.788 | −0.238 |
| PTHLH | −0.1025 | 0.0925 | 0.3035 | 0.1375 | −0.0705 | 0.0475 | 0.1945 | 0.2635 | 0.1255 | 9.0005 |
| PTK6 | −0.771 | −0.51 | −0.497 | −0.309 | 0.004 | −0.138 | −0.123 | −0.249 | 0.021 | 0.252 |
| PTPN4 | 0.141 | 0.881 | −0.22 | 0.384 | 0.568 | 0.407 | −0.392 | −0.67 | −0.213 | 0.232 |
| PTPN7 | 0.127 | −0.59 | −0.495 | −0.146 | 1.513 | 1.249 | −0.511 | −0.704 | −0.622 | 0.32 |
| PTPRF | 1.353 | 0.343 | 2.127 | −0.088 | 0.985 | 1.774 | 1.046 | 1.971 | 1.594 | −0.099 |
| PTPRN2 | −0.8825 | 0.0325 | 0.3585 | −0.2945 | −0.3145 | 0.0125 | −0.1605 | 0.2775 | 0.2055 | 0.1285 |
| PTPRT | −0.3375 | −0.9635 | −0.6185 | −0.6225 | −0.3905 | −1.1445 | −0.3505 | −0.4975 | −0.4455 | −0.3785 |
| PTS | 0.222 | −0.324 | −0.326 | −0.086 | −0.616 | −0.559 | −0.028 | 0.736 | −0.092 | −0.255 |
| PTTG1 | 0.2895 | 0.2835 | 0.2165 | 0.6845 | −1.7095 | −0.2675 | −0.1235 | 0.0425 | −0.0155 | −0.5285 |
| PURB | 0.8985 | 1.7065 | 1.2875 | 1.6885 | 1.4665 | 1.2215 | 1.9325 | 2.2955 | 1.6565 | −0.4455 |
| PXMP2 | −0.3525 | −0.1985 | −0.0495 | −1.0885 | 0.1865 | −0.1275 | 0.1025 | 0.1995 | −0.3605 | 0.1335 |
| PYCARD | −0.428 | 2.347 | 2.583 | 1.587 | −0.394 | 0.706 | 0.849 | 1.341 | −0.873 | −0.162 |
| PYCR1 | 0.711 | 0.693 | −0.597 | 1.152 | −0.675 | −1.165 | 0.259 | 0.465 | −0.773 | 0.436 |
| QDPR | 1.1265 | −0.2795 | 0.3215 | −0.7175 | 1.1705 | 1.0755 | 1.1445 | 1.1775 | 0.3215 | 1.5845 |
| QPRT | −0.447 | 0.407 | 1.750 | 1.461 | −0.964 | 0.104 | 0.022 | 0.958 | 2.665 | 1.832 |
| QSCN9 | 0.5395 | 0.1805 | 0.3785 | 0.0975 | −0.2685 | −0.2775 | −0.0155 | −0.0685 | 0.8195 | −0.5685 |
| RAB11FIP | 0.652 | 0.475 | 0.281 | −0.283 | 0.526 | −0.18 | 0.759 | 0.389 | −0.376 | 0.378 |
| RAB11FIP | −0.358 | −0.444 | −0.125 | 0.273 | −0.281 | −0.47 | −0.029 | −0.524 | −0.689 | 0.051 |
| RAB13 | 1.359 | 0.237 | 0.97 | 2.13 | 0.106 | 2.119 | 1.796 | 2.027 | 1.055 | 0.822 |
| RAB27A | 0.582 | −0.31 | 0.111 | −1.642 | −0.104 | 0.19 | 0.321 | 0.244 | −0.383 | 0.12 |
| RAB30 | −0.1905 | −0.2685 | −0.2145 | 0.2125 | 0.5255 | −0.1075 | 0.0205 | −0.0555 | −0.1495 | −0.2035 |
| RAB34 | 0.151 | −0.07 | 0.339 | −0.878 | −0.383 | −0.555 | −0.975 | −0.435 | −0.852 | 0.576 |
| RAB35 | 0.08 | −0.31 | −0.25 | −0.122 | −0.388 | −0.427 | 0.228 | 0.015 | 0.096 | 0.249 |
| RAB39B | −0.1685 | 0.2375 | 0.8215 | 0.5215 | −0.1365 | −0.7925 | 0.1095 | 0.1835 | 0.6035 | 0.0365 |
| RAB3A | 0.48 | −1.145 | 3.016 | 3.008 | 2.217 | 1.944 | 0.539 | 1.139 | 0.378 | −0.729 |
| RAB3IP | −0.146 | −0.105 | −0.008 | 0.59 | −0.246 | −0.383 | 0.011 | 0.098 | −1.076 | 0.231 |
| RABEP1 | −0.563 | −1.058 | −0.104 | −0.423 | −1.388 | −1.416 | −0.632 | −0.111 | −0.307 | −1.13 |
| RABGAP1 | 0.0465 | 0.4515 | 0.1645 | 0.0545 | −0.2375 | 0.2135 | 0.1055 | 0.3845 | 0.4015 | 0.3785 |
| RACGAP1 | −1.3225 | −1.2065 | −0.1995 | −2.2125 | −1.6005 | −1.6695 | −1.3575 | −1.4735 | 0.4475 | −0.8255 |
| RAD17 | −0.18 | −0.456 | −0.418 | −1.432 | −0.739 | −1.019 | −0.446 | −0.252 | −0.022 | −0.539 |
| RAD51 | −0.064 | 0.255 | −0.486 | 0.354 | −0.13 | −0.413 | −0.45 | −0.209 | −0.058 | 0.289 |
| RAD51AP | −0.1745 | −1.1995 | −0.1395 | −0.9805 | −0.6785 | −1.0535 | 0.0165 | −0.7395 | −0.0855 | −0.9425 |
| RAD52 | −0.156 | 0.204 | 0.241 | −1.211 | −1.338 | −1.209 | −0.271 | −0.336 | −1.097 | −0.941 |
| RAD54L | −0.3665 | −0.3235 | −0.0155 | −0.5845 | −0.3055 | −0.2155 | −0.0835 | −0.9845 | −0.7105 | −0.1135 |
| RAMP | −1.5705 | 0.2385 | −1.0855 | −0.8385 | −2.1935 | −2.4725 | −1.1115 | −1.6575 | −1.6535 | −1.2145 |
| RANBP1 | 0.8005 | 0.1305 | −0.0125 | 0.6145 | −0.5235 | 0.1745 | −0.1925 | 0.1105 | 0.7005 | 0.1455 |
| RARRES1 | 1.262 | −0.355 | 0.375 | −0.34 | −0.356 | 1.517 | 0.383 | 0.357 | 0.118 | 0.47 |
| RBBP7 | −0.0135 | 0.2475 | 0.1425 | −0.2965 | −0.2335 | −0.4715 | −0.0455 | −0.1735 | −0.3375 | −0.6455 |
| RBBP8 | 0.0655 | −0.1965 | 0.0635 | 0.1125 | −1.0595 | −0.0575 | −0.1815 | −0.1105 | −0.2015 | 0.4295 |
| RBM8A | −0.682 | 2.034 | 0.538 | 0.297 | 0.629 | 0.642 | −0.487 | −0.897 | 2.675 | 0.38 |
| RBX1 | 0.457 | −0.156 | 0.033 | −0.53 | −0.68 | −0.12 | −0.097 | −0.109 | −0.217 | −0.065 |
| RDHE2 | 0.021 | 0.412 | −0.2 | −0.838 | −0.516 | 0.075 | −0.702 | −0.886 | 0.237 | 0.543 |
| REA | −0.378 | −0.099 | −0.072 | −0.622 | 0.071 | 0.002 | −0.053 | −0.79 | −0.137 | 0.058 |
| REC14 | −0.4635 | −0.5725 | 0.2825 | −0.8955 | 0.2245 | −0.1085 | −0.0045 | −0.4575 | 0.2895 | 0.2295 |
| REG1A | −0.201 | −0.505 | 0.103 | −0.715 | −0.134 | −0.01 | −0.243 | −0.347 | 0.472 | −0.014 |
| RELB | 1.238 | −0.245 | 0.264 | 2.053 | 0.285 | 1.687 | 0.273 | 1.702 | 0.048 | 0.438 |
| REPIN1 | 0.205 | 1.342 | 0.833 | 0.995 | 0.17 | 1.668 | 1.267 | 1.994 | 0.643 | 1.209 |
| REPS2 | −0.5465 | 0.1185 | −0.1595 | 0.0895 | −0.9225 | −0.9555 | −0.4435 | −0.4385 | −0.4475 | −0.6525 |
| RERG | −0.1875 | 0.0545 | 0.2335 | 0.4255 | 0.3605 | 0.5905 | 0.7185 | 0.9475 | 0.0845 | −0.3195 |
| RFC4 | −0.5565 | 0.7325 | 0.2175 | −0.3225 | −0.5405 | 0.0145 | −0.0425 | 0.0265 | 0.0345 | −0.4925 |
| RGS10 | −0.0475 | −0.6195 | 0.6115 | −0.1415 | −0.7925 | −0.5495 | −0.4475 | −0.4755 | 0.0265 | −0.2395 |
| RH8DL2 | −0.427 | −0.362 | 0.074 | −0.764 | −0.12 | −0.188 | 0.179 | 0.291 | 0.776 | 0.124 |
| RIBC2 | 0.03 | 0.07 | 0.328 | −0.517 | −0.202 | −0.764 | −0.26 | −0.152 | −0.707 | −0.309 |
| RIC-8 | 0.614 | −0.406 | 3.642 | 0.506 | −1.145 | 1.392 | 0.911 | 0.956 | 5.316 | 2.634 |
| RIT1 | 0.185 | 0.077 | 0.384 | 0.594 | 0.765 | 0.45 | 0.556 | 0.457 | 0.22 | 0.548 |
| RLN2 | −0.309 | −0.064 | 0.246 | −0.231 | 0.106 | −0.039 | 0.375 | 0.55 | 0.43 | −0.882 |
| RNF10 | −0.5995 | 0.2385 | −0.0515 | 0.8115 | −0.4045 | −0.0395 | 0.1185 | −0.1735 | 0.6525 | 0.1615 |
| RNF135 | −0.3575 | −0.8515 | −0.6305 | −0.5095 | −0.6295 | −0.9875 | −0.1025 | −1.1905 | −0.5115 | −1.0045 |
| RNF7 | 0.575 | −0.167 | 0.194 | 0.724 | −0.53 | 0.324 | 0.008 | 0.418 | −0.383 | −0.242 |
| ROPN1 | 0.9485 | −1.4645 | 0.8335 | −0.4405 | −0.7525 | 1.9825 | 0.4485 | 0.2135 | −0.8365 | −0.4085 |
| RPA3 | 0.2085 | 0.2695 | 0.0275 | −0.2075 | 0.0705 | 0.0235 | −0.0505 | −1.1075 | −0.3225 | 0.0245 |
| RPESP | −0.035 | −0.347 | 0.132 | 0.249 | −0.461 | −0.494 | 0.371 | −0.971 | 0.158 | −0.108 |
| RPL11 | −0.019 | 0.021 | 0.619 | 0.082 | −0.427 | −0.304 | 0.372 | −0.364 | −0.81 | −0.701 |
| RPL13 | −0.645 | −0.187 | 0.09 | −0.954 | −0.91 | −0.313 | −0.224 | −0.066 | 0.202 | −0.45 |
| RPL13A | −0.4525 | −0.1315 | 0.3345 | 0.1365 | −0.4975 | −0.2915 | −0.0505 | −0.3725 | −0.5815 | −0.8485 |
| RPL15 | −0.109 | −0.204 | −0.268 | −0.642 | 0.161 | −0.226 | −0.255 | −0.039 | −0.27 | −0.064 |
| RPL18A | −0.331 | 0.859 | 0.2 | −0.748 | −1.402 | −0.534 | −0.011 | −0.474 | −0.576 | −0.046 |
| RPL19 | 0.016 | −0.122 | 0.203 | −0.692 | −0.287 | −0.119 | −0.077 | −0.335 | −0.4 | −0.313 |
| RPL21 | 0.0065 | −0.1055 | −0.1765 | −0.7355 | −0.0215 | −0.2275 | −0.2095 | −0.2675 | −0.3785 | −0.3785 |
| RPL23A | 0.291 | −0.045 | 0.426 | −0.642 | −0.61 | −0.015 | 0.148 | 0.008 | −0.07 | −0.054 |
| RPL23A | 0.3855 | 0.2185 | 0.8435 | 0.3305 | 0.1035 | 0.0575 | 0.5585 | 0.0595 | −0.0045 | −0.1195 |
| RPL26L1 | 0.2125 | −0.1135 | 0.6435 | −0.0135 | 0.2155 | −0.0845 | 0.0505 | −0.6545 | −0.3405 | −0.6195 |
| RPL29 | −0.1235 | 0.2555 | 0.3675 | −0.0835 | −0.1725 | −0.0375 | 0.3475 | 0.1705 | −0.5345 | −0.7515 |
| RPL35 | −0.3755 | −0.0985 | 0.3575 | −0.5395 | −0.1055 | −0.2685 | 0.0435 | −0.3205 | −0.4855 | −0.1945 |
| RPL35AL | 0.7815 | −0.8195 | 0.1195 | −0.2625 | 0.4245 | −0.0505 | 0.1605 | −0.5585 | −0.2705 | −0.6465 |
| RPS16 | −0.1135 | 0.1355 | −0.1335 | −0.3735 | −0.7045 | −0.0135 | 0.6645 | 0.9815 | 0.4715 | −0.0965 |
| RPS4Y1 | −0.1215 | −0.4925 | −0.6618 | −1.1625 | −0.5595 | −1.2135 | −0.8715 | −1.4995 | −0.8835 | −0.2255 |
| RRAGA | −0.266 | 0.279 | 0.574 | −0.925 | −1.136 | −0.365 | 0.224 | −0.162 | −0.117 | −0.681 |
| RRAGD | −0.519 | −1.139 | −0.081 | −1.999 | −1.033 | −1.713 | −1.144 | −0.206 | −1.485 | −0.802 |
| RRM1 | 0 | 0.3 | 0.155 | −0.495 | 0.432 | 0.095 | 0.643 | 0.003 | 0.027 | −0.351 |
| RRM2 | 0.28 | −0.653 | −1.661 | −1.689 | −1.914 | −2.013 | −1.038 | −2.441 | −1.323 | −0.037 |
| RSAFD1 | −0.5805 | −0.5195 | −0.3605 | −0.5645 | −0.4065 | −0.5135 | −0.0475 | −0.2255 | 0.0845 | 0.3555 |
| RSNL2 | 0.0425 | 0.2625 | −0.2775 | −0.1255 | −0.9895 | −0.2385 | 0.2245 | 0.0085 | 0.2725 | −0.2205 |
| RSU1 | 0.173 | 0.097 | −0.183 | 0.126 | 0.298 | −0.434 | 0.023 | 0.322 | −0.182 | −0.313 |
| RTN4IP1 | 0.834 | 0.441 | 1.548 | 0.687 | 1.036 | 0.96 | 0.896 | 1.454 | 0.913 | 0.388 |
| S100A11 | −0.054 | 0.437 | 0.787 | 0.449 | 0.181 | 0.073 | 0.305 | 0.305 | 0.505 | 0 |
| S100A14 | −0.1695 | 0.2045 | −1.5555 | −1.4935 | 1.0505 | −2.6316 | −1.0675 | −0.8735 | −1.3265 | −0.4955 |
| S100A16 | −0.2955 | −0.6105 | −0.2555 | −0.3575 | −0.4595 | −0.2935 | −0.1485 | −0.1795 | 0.4065 | −0.0135 |
| S100A9 | 0.894 | 1.358 | −0.016 | 0.115 | −0.608 | 0.483 | 1.434 | 1.2 | 0.24 | 0.502 |
| SAE1 | 0.0535 | −0.0605 | −0.0395 | 0.7475 | 0.3495 | 0.2545 | 0.4705 | 0.3125 | −0.4275 | −0.3325 |
| SALL2 | 0.004 | 0.741 | 0.046 | 0.547 | −0.633 | 0.429 | 0.095 | 0.795 | −0.262 | −0.453 |
| SARA1 | 0.2105 | 0.2725 | 0.4375 | 1.1445 | 1.1405 | 0.2585 | −0.0915 | −0.0485 | 0.5175 | 0.6035 |
| SARA2 | −0.262 | 0.143 | 0.335 | −0.731 | 1.291 | 0.041 | −0.435 | 0.196 | 0.269 | 2.71 |
| SAS | −0.884 | −0.012 | 1.411 | 0.486 | −0.183 | −0.317 | 0.038 | 0.487 | 0.693 | 0.871 |
| SCIN | −0.4475 | 0.3285 | −0.2125 | −1.4755 | −0.7885 | −0.2285 | 0.4725 | −0.3725 | −0.2705 | −0.7265 |
| SCML1 | −0.163 | −0.55 | −0.157 | −1.304 | 0.182 | −0.186 | −0.173 | 0.004 | 1.342 | 0.609 |
| SCPEP1 | −1.3265 | 0.2765 | 0.3815 | −2.1425 | −0.0155 | −0.7745 | −0.0595 | −0.3395 | 0.8695 | −0.0815 |
| SCRIB | −0.526 | −0.57 | −0.753 | −0.987 | 0.042 | −0.54 | −0.414 | −0.912 | −0.754 | −0.775 |
| SDC1 | −0.25 | 0.642 | 0.365 | −0.25 | −0.013 | −0.518 | −0.542 | −0.588 | −0.363 | −0.095 |
| SDC3 | −0.6295 | −0.6915 | −0.4725 | 0.3555 | −1.1145 | −0.5725 | −0.2415 | −0.1865 | −0.7955 | −0.3485 |
| SDCCAG0 | −0.0635 | −0.3135 | −0.1355 | −0.1015 | 0.1435 | 0.0335 | −0.5845 | −0.7685 | 0.8395 | 0.6555 |
| SEC61G | −0.11 | 0.184 | 0.165 | 0.128 | −0.234 | 0 | 0.077 | 0.51 | −0.367 | 0.038 |
| SECP43 | 0.8035 | 0.0775 | 0.6765 | 0.6925 | 2.4415 | 2.3835 | 0.5085 | 0.6875 | 0.8465 | 1.3945 |
| SEDLP | 0.423 | 1.977 | 0.901 | −0.986 | −0.919 | −0.602 | −0.142 | 0.874 | 1.821 | 0.577 |
| SEMA3B | −0.0526 | 0.0005 | −0.1375 | 0.9035 | −0.0005 | −1.0095 | 0.0095 | −0.4445 | 0.5885 | 0.7785 |
| SEMA3C | −0.0705 | 1.0085 | 0.1605 | 2.0055 | −1.1855 | 0.5585 | 0.2825 | 0.7925 | −0.0625 | −0.1916 |
| SEPW1 | 2.305 | −1.292 | 1.36 | 1.079 | −0.685 | 1.218 | 2.449 | 2.34 | 0.134 | 0.507 |
| SERP1 | −0.8385 | −1.5115 | −0.6385 | −1.7085 | −0.8295 | −0.7275 | −0.8015 | −1.8975 | −0.5835 | −0.0505 |
| SERPINA [symbol] | −1.214 | −1.902 | −1.395 | −1.783 | −1.896 | −1.718 | 0.536 | −1.018 | −1.689 | −0.399 |
| SERPINB [symbol] | 0.544 | −0.473 | −0.075 | 0.823 | 0.261 | −0.153 | 0.499 | 0.761 | 0.07 | 0.18 |
| SFRP1 | 0.137 | 1.05 | 0.185 | −0.411 | 0.007 | −0.431 | 0.512 | −0.617 | 0.565 | 1.218 |
| SFRS6 | −1.1465 | −0.9755 | −0.2515 | 0.9855 | −1.1915 | −1.4025 | −0.1105 | −1.2755 | 0.2555 | −0.2205 |
| SGCD | 1.1265 | 1.4005 | 0.0415 | 2.8075 | −0.2065 | 1.1335 | −0.2705 | −0.3575 | 0.2355 | 0.3285 |
| SGCE | −0.411 | −0.213 | −0.485 | −0.28 | −0.118 | −0.757 | −0.404 | −0.811 | −0.71 | −0.661 |
| SH3BGRL | −0.389 | 0.532 | −0.238 | 1.475 | 1.366 | 0.99 | 0.554 | 0.555 | 0.597 | −0.188 |
| SHFM1 | −0.0885 | −0.1695 | 0.3805 | 0.1775 | 0.1155 | −0.1126 | −0.0585 | −0.5595 | −0.0655 | −0.1225 |
| SIAT7B | −1.115 | −1.307 | −0.69 | −0.363 | −0.013 | −0.472 | −0.627 | 0.113 | −0.624 | −0.881 |
| SIGLEC12 | 0.196 | −0.351 | 0.041 | 0.166 | −0.111 | −0.388 | 0.477 | −0.258 | −0.583 | −0.316 |
| SIT | 0.3355 | 0.5375 | −0.1095 | 0.9665 | −1.0915 | −0.1205 | −0.2495 | −0.0365 | 0.0465 | 0.2485 |
| SITPEC | −0.0605 | −0.3405 | 0.0385 | −0.2885 | 0.8265 | −0.0175 | 0.3595 | 0.1365 | −0.0135 | 0.0125 |
| SKP1A | −0.0335 | −0.7875 | −1.1825 | −0.8435 | 0.4215 | −1.0615 | 0.2735 | −0.5525 | 0.1165 | 0.2275 |
| SLC12A9 | −0.028 | 1.499 | 2.155 | 0.436 | 1.477 | 1.657 | 0.909 | 1.46 | −0.904 | −0.77 |
| SLC13A3 | 1.1155 | 0.3145 | 1.4145 | 0.0065 | 1.2535 | 2.2185 | 0.6355 | 1.1405 | 0.8125 | 0.1435 |
| SLC16A6 | 1.9 | −1.335 | 2.985 | −3.635 | −0.839 | −0.968 | 0.156 | 0.458 | −1.524 | −0.305 |
| SLC19A2 | 0.0845 | 0.6145 | 1.5575 | 0.7995 | −0.1365 | 0.5355 | 1.0725 | 1.6895 | 0.2475 | 0.3355 |
| SLC1A1 | −1.5115 | −0.1605 | 0.4925 | 0.9425 | −0.7135 | 0.0425 | 0.8375 | 0.8665 | 0.7125 | 1.8305 |
| SLC1A2 | 0.3155 | 0.7795 | −0.1775 | 1.2625 | 2.0085 | 0.3755 | 0.5275 | 0.8725 | 0.3475 | −0.4425 |
| SLC24A3 | −0.196 | −0.227 | 0.288 | 0.913 | −0.553 | 0.026 | −0.183 | 0.257 | 0.041 | −0.654 |
| SLC25A29 | −1.002 | −0.08 | −0.379 | −0.087 | −1.252 | −0.858 | −0.116 | 0.19 | −0.312 | 0.063 |
| SLC25A4 | 1.297 | 2.828 | 2.654 | −0.799 | −0.185 | −0.926 | 2.416 | 2.541 | 0.06 | 3.453 |
| SLC25A5 | 0.4145 | −0.4385 | 0.6595 | −0.1935 | 0.2945 | −0.6775 | 0.3485 | 0.0035 | −0.0845 | −0.1075 |
| SLC27A2 | 0.172 | 0.05 | 0.058 | 4.834 | 3.745 | −0.199 | −0.391 | 0.004 | 0.019 | −0.039 |
| SLC27A5 | −0.173 | −0.778 | 0.188 | −1.208 | 0.23 | −0.444 | 0.003 | −0.109 | 1.041 | −0.671 |
| SLC30A8 | 1.2515 | 1.3735 | 1.3595 | 2.9775 | 2.0835 | 3.4285 | 2.5655 | 2.5435 | 0.7475 | 1.8035 |
| SLC39A4 | −1.108 | −0.846 | 1.868 | 0.026 | 0.325 | −1.564 | 0.869 | −1.399 | 0.103 | −0.697 |
| SLC39A6 | 0.0395 | −0.8165 | 0.1075 | −0.5055 | 0.1125 | −0.1485 | 0.0235 | −0.5445 | −0.3075 | −0.5935 |
| SLC40A1 | −1.0245 | −0.3995 | 0.6295 | −1.5805 | −0.3365 | −0.9835 | −0.4165 | −0.1205 | 0.5385 | −0.5475 |
| SLC41A3 | −0.084 | −0.392 | 3.376 | 0.564 | −0.161 | 0.303 | 1.35 | 1.791 | −0.824 | −0.086 |
| SLC5A6 | 0.4405 | −0.5425 | 0.5195 | 0.9675 | 0.1445 | 0.6395 | 0.5565 | 0.8565 | 1.2405 | 0.8315 |
| SLC6A4 | 4.7735 | 4.2585 | 3.9265 | −2.2315 | −0.6475 | 0.0805 | 1.8925 | −0.8735 | −2.1545 | −0.3205 |
| SLC9A3R1 | 1.2815 | 0.6575 | 3.5365 | 0.5615 | −0.5085 | 0.9875 | 1.6885 | 2.5655 | 1.2295 | −1.1815 |
| SLITRK6 | 0.07 | −0.137 | 0.283 | −0.505 | −0.79 | −0.425 | −0.307 | −0.249 | −0.006 | −0.195 |
| SMA3 | −0.378 | −0.216 | −0.575 | −1.145 | −0.089 | −0.298 | −0.014 | −0.895 | −0.557 | 0.618 |
| SMAD2 | 0.795 | 0.226 | −0.498 | −0.236 | −0.706 | −0.467 | −0.084 | −0.045 | −0.407 | 0.259 |
| SMO | 0.157 | 0.008 | 0.239 | 0.383 | 0.778 | 0.021 | 0.253 | 1.596 | −0.318 | 0.033 |
| SMU1 | −0.248 | −0.235 | −0.39 | 0.051 | −0.312 | −0.415 | −0.009 | −0.138 | 0.18 | −0.142 |
| SNPH | −0.055 | −0.139 | −0.163 | −0.006 | −0.326 | −0.426 | 0.058 | −0.303 | −0.387 | −0.199 |
| SNRPB2 | 0.0875 | 0.0745 | −0.1055 | −0.0745 | −0.3725 | 0.0355 | −0.0885 | −0.5035 | −0.2725 | −0.1415 |
| SNRPD1 | 0.299 | 0.176 | −0.135 | 0.289 | −0.47 | −0.207 | −0.421 | 0.118 | −0.385 | −0.447 |
| SNRPD3 | −0.5305 | 0.4215 | 0.1715 | −0.3175 | −1.7195 | −1.5325 | 0.0835 | −0.3445 | 0.5465 | 0.2565 |
| SNRPG | −0.0015 | −1.0395 | −0.1605 | −0.8385 | −0.7815 | −1.0315 | −0.5235 | −0.4725 | −0.4785 | −0.6315 |
| SNX7 | −0.0035 | 0.1925 | 0.6945 | −0.2565 | 1.6525 | 1.7685 | −0.1255 | −0.5525 | −0.3215 | −0.4165 |
| SO02 | −0.4825 | −0.0475 | −0.0155 | 0.6245 | 0.5345 | −0.3785 | 0.4705 | −0.4805 | −0.0085 | −0.5155 |
| SORCS1 | −0.407 | 1.78 | 1.006 | 0.361 | 0.468 | −1.101 | 0.525 | 0.535 | −0.876 | −0.942 |
| SOX4 | −0.1085 | 0.7965 | −2.0325 | 0.3845 | −1.8245 | −2.0685 | −0.0235 | −0.7325 | 1.3325 | 1.2435 |
| SOX9 | −0.669 | −0.9 | −0.187 | −1.019 | −0.352 | −1.272 | −0.734 | −0.588 | −0.576 | −0.94 |
| SPAG18 | −0.3445 | 0.2335 | 0.2775 | −0.7445 | −0.9075 | −0.2595 | −0.2875 | −0.7115 | −0.2105 | −0.1095 |
| SPAG5 | 0.4295 | 0.2255 | 0.0025 | 0.1385 | 0.8445 | 0.3175 | 0.8275 | 0.3395 | 0.5885 | 0.7375 |
| SPC18 | −0.301 | 0.187 | −0.195 | −0.725 | −0.378 | 0.863 | 0.28 | 0.174 | −0.004 | 0.299 |
| SPFH2 | −0.03 | 0.063 | −0.147 | 0.556 | −0.2 | 0.163 | 0.121 | −0.037 | 0.417 | 0.429 |
| SPG3A | −0.945 | 0.275 | −0.325 | −2.025 | −1.635 | −1.845 | −1.225 | −0.576 | 0.075 | −0.155 |
| SPPL3 | −0.137 | 0.192 | −0.18 | 0.197 | −0.082 | 0.59 | −0.158 | 0.019 | 0.167 | 0 |
| SQLE | 1.174 | 0.138 | 2.01 | −1.184 | 1.196 | 0.815 | −0.619 | 0.031 | 1.428 | −0.092 |
| SQRDL | −0.3595 | 0.6475 | 0.0035 | −0.8705 | −1.1775 | −0.2265 | −0.0815 | −0.0145 | 0.7755 | −0.0175 |
| SREBF1 | −0.3805 | −0.0625 | −0.0215 | −0.7965 | −0.6805 | −0.4095 | −0.5585 | −0.1515 | −0.1365 | −1.0735 |
| SRI | 2.5655 | −0.0315 | −0.3535 | −0.4025 | 2.0685 | 3.6315 | 0.0195 | −0.4365 | −0.1955 | 0.3155 |
| SRPK1 | 0.2665 | −0.1805 | −0.0015 | −0.5325 | −0.3485 | −0.0265 | 0.3105 | 0.5205 | −0.9035 | −0.7735 |
| SSFA2 | −0.5435 | 0.3215 | −0.1785 | 0.2175 | −0.8795 | −0.6235 | −0.7345 | −0.0355 | −0.7415 | −0.5085 |
| SSR4 | −0.1155 | 0.1095 | −0.1285 | −0.1165 | −0.9345 | −0.1005 | 0.5365 | 0.4535 | 0.1435 | 0.4385 |
| STARD3NI | −0.9225 | 0.2885 | −0.4775 | 0.5245 | −1.4515 | −0.5195 | −0.8735 | 0.0165 | 0.0965 | −0.4125 |
| STARD7 | 2.5085 | 2.5005 | 0.4845 | 2.4095 | 3.0925 | 3.4115 | 3.3005 | 3.4675 | 4.2545 | 2.6105 |
| STAT1 | −0.3005 | 0.2825 | 0.3395 | −0.9005 | −0.8095 | 0.1285 | −0.1215 | 0.1795 | 0.2765 | −0.3935 |
| STC2 | −0.2935 | −0.6455 | −0.3375 | −0.2425 | −0.2075 | −0.5685 | −0.2325 | −0.2265 | −0.1625 | −0.6965 |
| STK24 | −0.4065 | −0.8635 | −0.2885 | −0.6425 | −1.6485 | −1.4585 | −1.0965 | −1.0115 | −0.2285 | −0.9145 |
| STK6 | 0.063 | −0.184 | −0.024 | −0.847 | 0.347 | −0.223 | −0.364 | −0.737 | 0.079 | −0.332 |
| STMN1 | −0.1125 | −0.1845 | −0.2715 | −0.3865 | 0.1285 | −0.2155 | −0.2845 | −0.4735 | 0.2265 | −0.1795 |
| STX1A | 0.4845 | 0.6335 | −0.8285 | −0.2585 | −1.7025 | −0.9525 | −0.6695 | −0.2405 | −0.6785 | −0.2025 |
| STX3A | 0.0485 | −0.1165 | −0.0775 | −0.4965 | 0.3775 | 0.0445 | 0.0235 | 0.1315 | −0.1505 | 0.0716 |
| STYX | 0.4425 | 0.7785 | 0.4055 | 0.7725 | −0.1975 | 0.1695 | 1.1235 | 0.9875 | 1.7345 | 0.8585 |
| SUGT1 | −0.433 | −0.162 | −0.556 | −0.666 | −0.696 | −0.328 | −0.357 | −0.582 | 0.008 | 0.2 |
| SUMF1 | −0.3225 | −0.2965 | −0.0285 | 0.1035 | −2.2555 | −1.2175 | −0.3085 | 0.2965 | −0.3985 | −0.3545 |
| SUMO2 | 0.3385 | −0.2585 | −0.3165 | −0.6485 | 0.0855 | −0.4405 | −0.3355 | 0.0375 | −0.0205 | −0.4095 |
| SUV39H2 | 0.2455 | 1.6905 | 0.4355 | 0.2205 | 0.4665 | 0.2055 | −0.2455 | 0.0905 | 0.4155 | −0.3165 |
| SYAP1 | −0.5845 | 0.2655 | 0.1005 | −1.3875 | −0.3965 | 0.5365 | 0.5035 | −0.2485 | −0.7275 | −0.7435 |
| SYCP2 | 0.511 | 1.731 | −0.608 | 2.216 | 2.261 | 1.857 | 0.835 | 0.631 | 0.763 | 1.8 |
| SYNGR1 | −0.2245 | −0.0285 | 0.4265 | −0.3645 | −0.2415 | 0.2555 | 0.0155 | 0.3625 | 0.4715 | −0.0315 |
| SYTL2 | 0.1575 | −0.1945 | 0.2035 | −0.3395 | 0.1085 | −0.2545 | −0.0075 | −0.1935 | 0.0435 | 0.0505 |
| TAF18 | 0.5365 | 0.4465 | 0.3825 | −0.3435 | 1.2915 | 0.5505 | 0.1035 | 0.3215 | −0.3225 | −0.5815 |
| TAF8 | −0.057 | −0.345 | −0.194 | −0.467 | −0.394 | −0.411 | −0.242 | 0.002 | 0.055 | 0.173 |
| TARBP1 | −0.795 | −0.504 | −0.494 | −0.562 | −1.496 | −0.772 | −0.742 | −0.798 | 0.537 | −0.294 |
| TARS | 0.1575 | 0.0695 | 0.3265 | 0.0335 | −0.0725 | 0.9995 | 0.2645 | 0.7575 | 0.6645 | 0.4525 |
| TATDN1 | 0.1335 | −0.0495 | 0.0745 | −1.4855 | −0.2375 | 0.1255 | −0.7065 | 0.0415 | 0.3005 | 0.3285 |
| TBC1D12 | −0.1335 | 0.2815 | −0.1285 | 0.5065 | 1.4055 | −0.0365 | 0.2925 | 0.6405 | 0.2825 | −0.5085 |
| TBC1D14 | 0.636 | −0.151 | −0.235 | 0.728 | −0.007 | 0.24 | −0.292 | −0.009 | −0.137 | −0.216 |
| TBC1D3 | 1.1205 | −2.6775 | −1.6915 | −0.2265 | 1.3075 | 2.2125 | 0.2295 | −0.1735 | 1.6895 | 2.2285 |
| TBCA | −1.133 | 0.304 | 1.019 | 0.617 | −0.463 | −0.921 | −0.271 | −0.945 | 1.759 | 1.983 |
| TBX1 | −0.3885 | −0.3345 | −0.5285 | −0.5625 | −0.4435 | −0.6345 | −0.3525 | −0.5135 | 0.0275 | −0.1705 |
| TBX3 | −0.17 | 0.21 | 0.238 | 1.813 | −0.387 | 0.745 | 0.498 | 0.87 | 0.004 | 0.091 |
| TCAP | 0.078 | −0.03 | 0.056 | 2.028 | −0.587 | 0.805 | 0.699 | 0.873 | 0.017 | −0.399 |
| TCEAL1 | 0.3035 | 0.0605 | −0.6805 | 2.1075 | −0.2445 | 0.7655 | 1.0435 | 0.9125 | 0.4865 | 1.2045 |
| TCEAL3 | −0.256 | −0.284 | −0.04 | −1.088 | 1.28 | −1.185 | −0.412 | 0.16 | 1.098 | 1.116 |
| TCEAL4 | −0.426 | −0.082 | 0.379 | −0.014 | 0.261 | 0.138 | 0.15 | 0.292 | −0.076 | −0.357 |
| TCF15 | 0.097 | −0.352 | 0.464 | −0.998 | 0.412 | 0.002 | 0.761 | 0.361 | −0.064 | −0.121 |
| TCF3 | 0.05 | 0.072 | −0.098 | 0.277 | 0.183 | 0.207 | −0.431 | 0.077 | 0.005 | −0.295 |
| TCF7L2 | −0.0075 | 0.1815 | −0.0595 | 0.9845 | 0.6215 | 0.4795 | −0.0475 | −0.2395 | 0.7615 | 0.6095 |
| TDRD7 | −2.8975 | 1.8505 | 1.3845 | 1.2005 | 0.6395 | −3.5055 | 0.7045 | 1.2015 | 0.7925 | −3.5405 |
| TESK2 | −1.643 | −0.993 | −0.188 | 0.788 | −0.949 | −0.931 | 0.559 | 0.019 | 1.043 | 0.565 |
| TFAP2B | −0.506 | 0.224 | −0.181 | 0.385 | 0.419 | 0.308 | −0.181 | 0.165 | 0.623 | −1.136 |
| TFCP2L1 | 2.0025 | −0.3585 | 0.4745 | 1.5585 | 2.2995 | 2.9215 | 2.0255 | 3.2765 | 1.5695 | 4.3005 |
| TFCP2L2 | 3.0305 | 0.0295 | −0.5245 | 2.9745 | 2.3525 | 2.5575 | 1.6245 | 2.1415 | 1.8685 | 3.7205 |
| TFF1 | −1.0435 | −0.3005 | 0.2875 | −0.6035 | −1.5785 | −1.1705 | −0.8755 | −0.9815 | 0.5165 | −0.3215 |
| TFF3 | 2.76 | −0.55 | −1.334 | −0.694 | −0.895 | 0.418 | 1.712 | −0.604 | −0.902 | 0.431 |
| TFRC | −0.2155 | 0.1755 | 0.0365 | −0.2345 | −0.2745 | 0.2685 | 0.1805 | −0.0555 | −0.2945 | −0.1425 |
| TGFBR3 | −0.341 | −0.323 | −0.156 | 0.573 | −0.399 | −0.53 | 0.198 | −0.244 | 0.281 | −0.833 |
| THY28 | −0.1165 | −0.5285 | 0.2175 | 0.8315 | −0.4605 | −0.1395 | 0.0885 | −0.4005 | −0.5425 | −0.8445 |
| TIF1 | 0.376 | 0.472 | −0.051 | 1.366 | −0.664 | 0.586 | 0.277 | 1.33 | 0.395 | −0.671 |
| TIGA1 | −0.61 | −0.775 | 0.142 | 0.127 | −0.622 | −0.555 | 0.315 | 0.514 | 0.099 | −0.172 |
| TIM14 | 0.1175 | 0.3325 | 0.3585 | −0.0025 | −0.4025 | −0.3485 | −0.0945 | 0.1155 | −0.4705 | −0.7875 |
| TIMELES [symbol] | 0.0835 | 0.2145 | 0.0035 | 0.1385 | −0.8076 | 0.3315 | 0.7675 | 0.6485 | −0.2775 | −0.0405 |
| TIMM9 | 1.408 | −0.469 | −1.035 | −0.667 | 1.078 | −0.294 | −0.306 | −1.412 | −1.492 | 0.229 |
| TINP1 | −0.3175 | −0.1855 | 0.5175 | −1.2135 | −0.8115 | −0.6785 | 0.2315 | 0.3565 | 0.1685 | −1.3116 |
| [symbol] IP1208 | 0.484 | 0.693 | −0.503 | 1.13 | 0.956 | 0.438 | 0.104 | 0.795 | −0.255 | 0.229 |
| TK1 | −0.4 | 1.774 | 0.432 | −0.218 | 0.134 | 0.051 | 0.894 | 0.675 | 0.413 | −0.443 |
| TLE2 | −0.183 | −1.004 | −0.849 | 1.663 | −1.281 | 0.993 | −0.503 | 0.46 | −0.407 | 1.696 |
| TLR5 | −0.524 | 0.61 | −0.305 | 0.406 | −0.219 | 0.777 | 0.543 | 1.357 | 0.265 | 0.277 |
| TM4SF12 | 0.055 | 0.315 | −0.2 | −0.08 | −0.934 | −0.274 | −0.204 | 0.247 | 0.001 | −0.148 |
| TM4SF13 | 1.021 | 0.619 | 1.639 | 0.91 | 1.488 | 2.37 | 1.604 | 2.077 | 1.047 | 0.111 |
| TMEM14A | 0.4765 | 0.1245 | −0.0845 | 0.6706 | 0.0295 | 0.0185 | 0.1255 | 0.1545 | −0.3935 | 0.3725 |
| TMEM25 | −0.2785 | −0.7645 | −0.3345 | −1.0475 | 1.0145 | 0.9645 | 0.5285 | 0.1275 | −0.0755 | 0.1995 |
| TMEM4 | −1.378 | 0.32 | −0.427 | 0.179 | −0.127 | −0.745 | 0.066 | −0.354 | −0.121 | −0.301 |
| TMPRSS5 | −0.13 | −1.634 | −0.488 | −0.588 | −1.225 | −1.345 | −1.305 | −1.627 | −1.321 | −0.842 |
| TMSB10 | −0.835 | −1.487 | −1.45 | −0.765 | −1.085 | −1.534 | −0.149 | −0.3 | −1.77 | −1.659 |
| TMSNB | 0.4665 | 0.2215 | −0.8125 | −0.9145 | −0.9985 | −1.1905 | −0.1475 | −0.4475 | −0.7055 | 0.0885 |
| TNFRSF1 [symbol] | 0.056 | −0.002 | −1.083 | −1.401 | −0.317 | −1.532 | 0.002 | −0.249 | −0.965 | −0.127 |
| TNFRSF2 [symbol] | −0.8076 | 0.3665 | −0.3545 | −0.6195 | 0.3725 | −0.7055 | −1.0855 | −1.1825 | 0.2425 | 0.3905 |
| TNFRSF2 [symbol] | −0.0855 | 1.2855 | 1.6335 | −0.6216 | 3.7195 | 0.0855 | 1.3225 | 1.0755 | −0.4505 | −0.0755 |
| TNFSF4 | 0.3905 | 1.4355 | 0.2315 | 0.5595 | 1.1185 | 1.6625 | 0.4715 | 0.7395 | 0.7555 | −0.1925 |
| TNFSF7 | 1.0905 | −0.7705 | −1.5115 | −0.8035 | 0.3945 | −0.1975 | 0.3285 | 0.4635 | −0.3705 | 0.2255 |
| TNIK | 0.2205 | −0.1565 | −0.5585 | −0.4445 | −0.2165 | −0.5445 | −0.4205 | −1.1205 | −0.4375 | −0.0615 |
| TNNC2 | 0.342 | 2.684 | 0.351 | 1.403 | −0.543 | −1.193 | −0.632 | −1.347 | 0.785 | 0.808 |
| TNNI2 | −0.246 | 0.701 | 0.036 | 1.016 | 0.521 | −0.052 | −0.055 | 0.199 | 0.729 | −0.227 |
| TNNI3 | 0.1576 | 0.6585 | 0.6085 | −0.2045 | 0.2285 | 0.8285 | 0.2445 | 0.4825 | 0.1415 | −0.8685 |
| TOB1 | 0.6065 | −0.3025 | 0.1375 | −0.1185 | −0.8165 | −0.3305 | 0.1415 | −0.3935 | −0.6465 | 0.0795 |
| TOM1L1 | −0.5695 | −1.0745 | 0.1265 | −1.2685 | 0.1075 | −0.4475 | 0.0155 | −0.6545 | 0.4095 | 0.1325 |
| TOMM7 | −0.4545 | −0.8855 | 0.3255 | −0.8485 | −0.7995 | −0.9605 | −0.6055 | −0.0685 | 0.0325 | −1.0305 |
| TOP1MT | 0.278 | 0.649 | 0.197 | 0.613 | −0.481 | 0.225 | −0.108 | −0.12 | 0.175 | −0.365 |
| TOP2A | −0.3035 | 0.3985 | −0.0725 | 0.9265 | 0.0105 | −0.1915 | 0.3235 | 0.3405 | 0.1645 | −0.5115 |
| TOP2B | −0.667 | −0.877 | 8.378 | −1.95 | −1.888 | −1.588 | −0.755 | −0.001 | −1.135 | −2.612 |
| TOPBP1 | 0.0815 | 0.2385 | 0.7515 | 0.6675 | 0.3625 | 0.1515 | 0.9585 | 0.2305 | 0.5435 | −0.0765 |
| TOPK | −0.0625 | 0.9675 | 0.5355 | −1.1435 | −0.7365 | −0.5755 | −0.5375 | 0.0145 | −0.0975 | −0.6065 |
| TP53AP1 | 0.3345 | 0.7665 | 0.0725 | 1.6185 | 0.6585 | 0.4585 | 0.3905 | 0.5725 | 1.0575 | 1.0765 |
| TP53BP2 | −0.2785 | 1.5385 | −0.4885 | −2.9235 | −1.4725 | 0.9845 | 1.1055 | 1.9325 | 0.0875 | 0.1115 |
| TP53INP1 | −0.7525 | −0.1035 | −0.1665 | −0.6625 | −0.9285 | −0.9665 | −0.7765 | −0.5915 | −0.0765 | −0.6005 |
| TPD52L1 | −1.7 | −0.036 | 0.191 | 0.221 | 0.56 | −0.248 | −0.319 | 0.586 | −0.706 | −0.651 |
| TPRT | −0.5495 | −0.8815 | 0.4345 | 0.7025 | 0.6135 | 0.3485 | 0.5845 | 0.1705 | 0.0215 | −1.1795 |
| TRA@ | 1.23 | 0.05 | −0.038 | −0.315 | −0.084 | 0.687 | 0.461 | 0.378 | 0.423 | −0.091 |
| TRAF4 | 0.111 | −0.182 | 2.203 | 0.782 | 0.61 | 0.771 | 0.625 | 0.949 | 0.215 | −0.022 |
| TRAPPC4 | 0.341 | 0.299 | −0.614 | −0.057 | 0.383 | −0.435 | 1.726 | 0.846 | 0.081 | 0.1 |
| TRH | −0.5515 | −0.1235 | −0.7025 | −1.8405 | −1.4675 | −0.4278 | −0.7355 | −2.4885 | −1.1125 | −0.6875 |
| TRIM17 | 0.4385 | 0.8985 | 0.1685 | 0.2405 | −0.2365 | 0.0395 | 0.4825 | 0.5255 | 0.3245 | −0.3705 |
| TRIM2 | 0.1245 | 0.1845 | 0.0895 | 0.0505 | −0.8215 | −0.0716 | −0.0215 | −0.1615 | −0.0018 | −0.4815 |
| TRIM6 | 0.483 | 1.279 | 0.968 | 0.183 | −0.358 | 0.813 | 0.036 | 0.239 | 1.035 | 1.304 |
| TRIP3 | −0.1885 | 0.2675 | 0.0445 | 0.6815 | −0.4485 | 0.1655 | 0.1585 | 0.2545 | 0.1815 | −0.0825 |
| TRPS1 | 1.1845 | 1.1285 | 1.0105 | −0.2275 | 0.6685 | 0.9895 | 0.5685 | 0.8765 | 0.4345 | 0.7345 |
| TSG101 | 0.1275 | 1.7395 | 8.6825 | 0.4215 | 0.8705 | 0.8405 | 1.7435 | 1.9525 | −0.2335 | −0.9595 |
| TTC10 | −0.3605 | −0.8335 | 0.1675 | −0.4545 | −1.2775 | −1.3515 | −0.3025 | −0.0975 | −0.2255 | −1.5835 |
| TTC12 | 0.418 | −0.391 | 0.146 | −0.289 | −0.101 | −0.653 | 0.106 | −0.181 | −0.394 | 0.288 |
| TTK | −0.3475 | −0.7325 | −0.2885 | −0.7095 | −0.1875 | −0.6895 | −0.6765 | −1.2085 | −0.6905 | −0.9055 |
| TTLL4 | −0.256 | −0.188 | −0.219 | −0.435 | −0.082 | −0.586 | −0.12 | −1.208 | 0.125 | −0.428 |
| TTYH1 | 0.3835 | −0.3595 | 0.0825 | 0.3025 | 1.2255 | 0.2845 | 0.7655 | 0.2325 | −0.1745 | 0.6825 |
| TUBB3 | −0.1795 | −0.1285 | −0.5105 | 0.5805 | −0.6995 | −0.5915 | −0.0905 | −0.2875 | −0.1025 | −0.4475 |
| TULP3 | −1.192 | −0.735 | 0.315 | −1.875 | −1.705 | −1.215 | −1.025 | −0.515 | 0.735 | −1.245 |
| TXN | −0.365 | −0.135 | 0.283 | −0.491 | 0.5 | −0.088 | 0.59 | 0.042 | 0.452 | 0.114 |
| TYMS | −1.3115 | −1.4155 | 0.6405 | −1.0805 | −1.8065 | −1.7825 | −0.4845 | −1.5485 | 0.3125 | −0.6555 |
| U5-116KD | 0.4475 | 0.1215 | −0.0545 | 0.3735 | 0.0325 | 0.1055 | −0.0565 | 0.4375 | −0.0485 | 0.2205 |
| UBE2E3 | 0.265 | 0.157 | 8.792 | 0.13 | 0.54 | 0.334 | 0.71 | 0.897 | 1.221 | 0.631 |
| UBL5 | 0.1075 | −0.0715 | −0.2945 | −1.3385 | −0.3005 | −1.4995 | −0.7375 | −1.7885 | −0.2515 | 0.9755 |
| UBN1 | −0.316 | 1.178 | 0.35 | −0.8 | −1.025 | −0.069 | −0.155 | 0.541 | −0.055 | −1.221 |
| UCHL1 | −0.833 | −0.357 | 1.628 | 0.325 | 1.242 | 1.981 | 0.866 | 1.533 | 1.038 | 0.286 |
| UCHL3 | 0.118 | −0.026 | −0.139 | −0.17 | −0.194 | −0.414 | 0.112 | 0.252 | −0.753 | 0.461 |
| UGCG | 0.7755 | −0.0065 | −1.2215 | 4.0065 | −0.5515 | 7.0215 | 4.4225 | 0.9015 | 0.4255 | −0.5605 |
| UGCGL1 | 0.0335 | −0.5405 | −0.2515 | −0.5625 | 0.2015 | −0.7025 | −0.6445 | −0.7075 | 0.9805 | 0.3195 |
| UGT2B7 | −0.2075 | −0.0915 | −0.2195 | 0.4695 | −0.0675 | 0.3205 | 0.5865 | 0.3275 | 0.7835 | −0.1015 |
| UNC13D | −0.132 | 0.198 | 0.016 | 0.336 | −0.947 | 0.568 | −0.328 | −0.329 | 0.185 | 0.525 |
| VAMP8 | −0.001 | 0.749 | −0.253 | −0.274 | 0.047 | −0.038 | 0.018 | 0.456 | −0.318 | −0.18 |
| VAPA | 0.5815 | 0.3875 | 0.6315 | 0.0825 | 0.2795 | 0.9145 | 0.1425 | 0.1445 | 1.4855 | −1.1345 |
| VAV1 | 1.038 | −0.425 | −1.141 | 0.937 | 0.365 | −0.953 | −0.391 | −1.897 | 0.426 | 1.065 |
| VAV3 | 0.132 | −0.099 | −0.184 | −0.349 | −0.915 | −0.369 | −0.615 | −0.027 | −0.233 | −0.588 |
| VCX3 | 0.205 | −0.276 | 2.298 | −0.275 | −0.145 | 0.333 | −0.026 | 0.09 | −0.092 | 0.085 |
| VDAC2 | −0.1315 | −0.0615 | −0.3615 | −0.0315 | −0.3015 | −0.4115 | −0.2915 | −0.1115 | −0.1715 | −0.3815 |
| VEST1 | −0.155 | −0.34 | 0.531 | 0.079 | 0.435 | 0.012 | 0.108 | −0.641 | −0.102 | −0.732 |
| VGLL1 | −0.2135 | 0.2015 | −0.7125 | 0.0575 | −0.1675 | 0.6425 | 0.6565 | 1.2205 | −0.5335 | −0.4605 |
| VMD2L2 | −0.239 | 0.152 | −0.061 | −0.915 | 1.349 | 0.329 | −0.035 | −0.452 | 0.022 | 0.284 |
| VSX1 | 0.361 | 0.589 | 0.292 | −0.554 | 0.068 | −0.298 | 0.154 | 0.4 | −0.132 | −0.492 |
| WARP | 0.298 | −1.053 | 0.323 | 0.276 | 1.195 | 0.434 | 0.484 | 0.569 | 0.76 | 0.161 |
| WDR26 | 0.1525 | 0.2145 | 0.3895 | 0.4645 | 0.8185 | 0.4925 | 0.0285 | 0.8255 | 0.8825 | 0.6655 |
| WDR6 | 0.769 | −0.623 | 0.961 | −0.683 | −0.089 | 0.572 | 1.049 | 1.358 | −0.536 | 0.777 |
| WHSC1L1 | −0.22 | −0.809 | −0.839 | −0.509 | −0.479 | −0.289 | −0.029 | −0.749 | 0.111 | −0.249 |
| WNT3 | −0.6835 | 0.7815 | 0.2825 | −0.7395 | −0.3385 | −0.2425 | −0.6455 | 0.1245 | −0.6145 | −0.4815 |
| WNT6 | 0.4505 | 0.1415 | 0.2035 | 1.2745 | 0.8275 | 1.3275 | 0.8445 | 1.0335 | 2.6105 | 1.6465 |
| WSB2 | −0.1935 | 0.0775 | −0.5805 | 0.4155 | −0.8205 | −0.4235 | −0.1505 | −0.5115 | −0.0245 | −0.0405 |
| WWP1 | 2.6815 | 0.9785 | −0.1685 | 0.0105 | 0.2655 | 1.1435 | 0.3325 | 0.7395 | 0.8565 | 0.7105 |
| WWP2 | 0.091 | −0.311 | −0.099 | −0.989 | 0.566 | −0.025 | 0.666 | 0.473 | −0.162 | 0.237 |
| XBP1 | −0.544 | −0.464 | 0.015 | −0.769 | −0.085 | −0.508 | −0.039 | 0.199 | 0.487 | 0.451 |
| XYLT2 | −0.0405 | −0.3125 | −0.2255 | −0.8825 | −0.3895 | −0.4385 | −0.0625 | 0.0475 | −0.0335 | 0.0725 |
| YBX2 | −0.592 | 0.828 | −0.001 | −0.086 | −0.955 | 0.211 | −0.399 | −0.39 | 0.666 | 0.05 |
| YIF1 | 0.6315 | 0.7945 | −0.0115 | 0.1115 | 0.1075 | 0.6825 | 0.8055 | 1.1895 | 0.7155 | −0.2165 |
| ZA20D3 | 0.2925 | −0.3555 | 0.4685 | −0.1255 | 0.3165 | 0.3685 | 1.1825 | 0.9665 | −0.5005 | 0.0525 |
| ZAP128 | −0.2585 | −0.2485 | 0.2895 | 0.3185 | 0.0585 | 0.9115 | 0.8825 | 0.5645 | 0.5915 | 0.9655 |
| ZBED3 | 0.256 | 0.023 | 0.275 | −0.578 | −0.167 | −0.35 | −0.031 | −0.513 | −0.474 | −0.353 |
| ZBTB4 | 0.021 | −0.168 | −0.888 | 0.182 | 0.365 | 0.153 | 0.519 | 0.568 | 0.126 | 0.312 |
| ZCWCC1 | −0.1605 | −0.4095 | −0.8825 | −0.2695 | −0.0395 | −0.2325 | −0.4185 | −0.3495 | −0.2985 | −0.0015 |
| ZCWCC2 | −0.452 | 0.103 | −0.607 | −1.004 | 1.148 | −0.215 | 0.448 | 0.785 | 0.16 | 0.839 |
| ZDHHC13 | −0.3985 | 0.8555 | 0.4345 | −1.2835 | 0.3715 | −0.4205 | 1.0155 | −0.9465 | 0.1745 | 2.0315 |
| ZDHHC14 | 0.3275 | 0.9175 | 0.1735 | 0.3195 | −0.0805 | 0.2965 | 0.4695 | 0.3815 | 1.1085 | 0.1715 |
| ZFHX4 | −0.2885 | −0.1375 | −0.0185 | −0.0195 | 0.5465 | 0.0255 | 0.3665 | 0.3715 | −0.2075 | −0.4225 |
| ZFYVE21 | 0.106 | −0.339 | −0.048 | 0.034 | −0.69 | −0.493 | −0.005 | 0.215 | −0.2 | −0.572 |
| ZNF133 | −0.196 | 0.33 | 0.432 | 0.376 | 0.185 | 0.285 | 0.101 | 0.255 | 0.366 | 0.377 |
| ZNF165 | 0.1765 | 0.1285 | −0.1095 | 0.1125 | −0.0045 | −0.1505 | 0.1515 | −0.0705 | 0.0185 | −0.1045 |
| ZNF24 | 0.503 | 0.634 | 0.481 | −0.436 | 0.222 | 0.112 | 0.031 | 0.769 | −0.112 | −0.213 |
| ZNF318 | −0.2915 | −0.0095 | −0.1855 | 0.1205 | 0.1135 | 0.0345 | −0.0835 | 0.2525 | −0.2335 | −0.1375 |
| ZNF54 | −1.1176 | 0.5745 | −1.4105 | −1.0275 | −1.1485 | −1.3255 | −0.1955 | −2.4695 | −1.4445 | 0.2045 |
| ZNF432 | −0.2025 | −1.1045 | 0.1175 | 0.3335 | −0.1905 | 0.0185 | −0.4345 | −0.1425 | −0.7185 | 0.0095 |
| ZNF482 | 1.781 | −0.591 | 0.975 | 1.704 | 1.4 | 2.301 | 0.755 | 1.651 | 1.077 | −1.031 |
| ZNF505 | 0.323 | −0.127 | 0.173 | 2.396 | 1.441 | 0.731 | 0.384 | 0.631 | −0.23 | −0.052 |
| ZNF552 | 0.7465 | −0.9515 | −0.1035 | 2.0035 | 2.0675 | 2.2315 | 1.3745 | 1.7605 | −0.0335 | −0.4885 |
| ZNF577 | −0.4575 | 0.0845 | 0.1955 | −0.5265 | 0.8775 | 0.0805 | −0.4025 | 0.0455 | 1.0145 | 0.6275 |
| ZNF587 | 0.3585 | −0.0785 | −0.0575 | 0.7365 | 0.2115 | 0.2875 | 0.4985 | 0.4855 | 0.5295 | −0.3865 |
| ZNF598 | 0.2245 | 0.5416 | 0.2025 | 0.8195 | 0.7215 | 0.6585 | 0.1525 | 0.2385 | 0.3965 | −0.4045 |
| ZNF599 | −0.063 | −0.385 | −0.09 | 0.736 | −0.211 | −0.062 | 0.195 | 0.243 | −0.187 | 0 |
| ZNF606 | −0.523 | −1.253 | −0.159 | 0.135 | −1.109 | −0.22 | −0.233 | −0.121 | 0.109 | −0.57 |
| ZNF614 | −0.282 | 0.007 | −0.333 | −0.492 | −0.001 | −0.076 | −0.075 | −0.168 | 1.479 | 0.577 |
| ZNF639 | | | | | | | | | | |
| ZNF7 | | | | | | | | | | |

| | Sample | | | | | | | | | | | | |
|---|---|---|---|---|---|---|---|---|---|---|---|---|---|
| | 9819-0103 | BR02-705 [symbol] | UB69 | UB21 | UB56 | UB44 | BC00040 | BR02-704 [symbol] | BR02-705 [symbol] | BR03-725 [symbol] | BR95-018 [symbol] | 9830-0200 | 9830-0201 |
| | Class | | | | | | | | | | | | |
| Symbol | LUMINAL | LUMINAL | LUMINAL | LUMINAL | LUMINAL | LUMINAL | LUMINAL | LUMINAL | LUMINAL | LUMINAL | LUMINAL | LUMINAL | LUMINAL |
| | 1.7595 | 0.8565 | 1.5065 | 0.6185 | 0.4315 | 1.0095 | 1.8705 | −0.8315 | −0.6865 | 2.2875 | −0.8215 | 1.6425 | 2.3855 |
| | 0.593 | 0.161 | −0.236 | 0.209 | 0.224 | 0.154 | 0.467 | 0.632 | 0.629 | 1.484 | 0.724 | −0.076 | −0.287 |
| ABAT | 1.884 | −0.97 | −1.477 | −1.983 | −0.995 | 0.555 | 2.988 | −0.775 | −0.134 | −0.22 | −2.458 | 2.788 | 2.013 |
| ABC1 | 1.0025 | −0.6415 | −0.3425 | −0.6115 | −0.1255 | 0.7695 | 0.3385 | −0.4355 | −0.4875 | −0.5545 | −1.3735 | 0.2265 | 2.8115 |
| ABCC11 | −0.9925 | 0.0965 | −0.7795 | −0.7805 | −1.3415 | −1.1555 | 1.3815 | 0.1925 | 0.2615 | 0.8815 | −0.8145 | 0.4335 | −0.2705 |
| ABCC3 | 0.178 | 0.057 | −0.216 | 0.218 | 0.731 | −0.72 | −1.026 | −0.527 | −0.528 | 1.618 | 0.114 | −0.103 | −0.867 |
| ACAS2L | 0.2435 | −0.1285 | −0.6135 | 0.0055 | 0.0355 | −0.3975 | −0.4575 | −0.4955 | −0.5175 | −0.1585 | 0.3645 | 0.3325 | −0.5465 |
| ACAT1 | −0.207 | 0.057 | −0.195 | 0.1 | −0.011 | 1.063 | −1.158 | −1.666 | −1.557 | 1.059 | −0.899 | 2.881 | 0.313 |
| ACAT2 | 0.7355 | 0.0575 | 0.3035 | −0.2225 | 1.5325 | −0.0055 | 1.6235 | −0.5655 | −0.4905 | −0.0135 | 0.2225 | 1.1415 | 0.5725 |
| ACOX2 | 1.8025 | 1.5025 | 1.5605 | 0.4365 | −0.2655 | 0.6545 | 1.6575 | 0.1295 | 0.0825 | 0.5985 | 0.1105 | −0.5775 | 0.8625 |
| ACDX3 | 0.1525 | −0.2445 | 0.2115 | 1.0655 | −0.0965 | 1.1615 | 0.3805 | −0.3645 | 0.4505 | 0.4705 | 0.2555 | −0.0905 | 1.6395 |
| ACSL3 | −0.661 | −0.881 | −0.899 | 0.029 | 0.389 | −0.048 | −1.35 | −0.88 | −0.744 | −0.29 | −0.997 | −0.69 | −0.555 |
| ACTA1 | −0.5105 | −0.0585 | 0.0855 | −0.1715 | −0.0085 | −0.1195 | −0.0305 | −0.2535 | −0.3275 | −0.4275 | 0.0735 | 0.5285 | −0.2355 |
| ACTB | −0.8965 | −0.4635 | −0.9945 | −0.0465 | −1.0715 | −0.1155 | −1.1005 | 2.8055 | 2.7515 | 1.6005 | −0.4295 | 1.7885 | 1.3085 |
| ADAM33 | −0.742 | 0.309 | 0.013 | −0.475 | −0.425 | 0.678 | −1.305 | 2.558 | 2.665 | −0.521 | −0.074 | −0.583 | 0.246 |
| ADAMDE [symbol] | 0.0285 | −0.1095 | −0.9535 | 0.3415 | 0.1745 | 0.0815 | 1.1735 | −0.3015 | −0.3825 | 1.0815 | 0.0935 | −0.5895 | 1.1905 |
| ADPN | −0.1185 | 0.1275 | −0.2735 | 0.6985 | 0.0245 | −0.4205 | 0.7395 | 1.7115 | 1.6415 | 1.0705 | 2.5685 | −0.3135 | −1.2705 |
| ADPRHL1 | 4.4715 | 2.7335 | 1.3575 | −0.7625 | −1.3255 | −0.1185 | 1.4435 | 3.1955 | 3.1945 | 1.7605 | 3.5325 | 2.3515 | 1.8935 |
| ADRA2C | −0.7555 | −1.6875 | −0.0855 | 0.2865 | 0.7545 | 0.4765 | 0.1325 | −0.6575 | −0.7565 | −0.9565 | −0.6535 | 0.5065 | −0.6345 |
| AGR2 | 2.3665 | −0.0535 | −0.1575 | 0.2475 | −0.1665 | −0.1405 | 2.1375 | 2.2935 | 2.4535 | 0.7475 | −0.1235 | −0.3635 | 1.7345 |
| AGT | 0.886 | 0.02 | 0.81 | 0.024 | 0.1 | 1.005 | −0.218 | −0.435 | −0.388 | 0.029 | −0.786 | 0.516 | 0.708 |
| AGTR1 | −0.758 | 1.425 | −0.173 | −0.319 | 0.826 | 0.762 | −0.039 | −0.086 | −0.142 | 0.342 | 0.453 | 0.749 | −0.109 |
| AHCYL1 | −0.078 | 0.478 | 0.742 | 0.556 | −0.837 | 0.875 | −1.402 | −0.74 | −0.765 | 0.658 | −1.125 | 0.248 | 0.613 |
| AHNAK | −0.537 | 1.056 | −0.887 | −0.442 | −0.385 | 0.472 | −1.25 | 0.211 | 0.273 | −0.298 | −0.115 | 1.058 | −0.684 |
| AHR | −0.2605 | −0.3305 | 0.0115 | −0.1445 | −0.0555 | −0.2815 | 0.3635 | 0.4045 | 0.4105 | 0.1625 | 0.4065 | −0.1855 | 0.0135 |
| AIM1 | 0.1715 | 0.5455 | 0.2735 | 0.3255 | −0.2425 | 0.6885 | −0.3535 | −0.1815 | −0.1175 | 0.1375 | 0.0635 | −0.4935 | 0.3395 |
| AK2 | 0.9305 | 0.4285 | −0.0095 | 2.5145 | 0.3015 | 0.5415 | −0.5305 | 0.0095 | −0.1375 | 0.0915 | −0.4025 | −0.9825 | −0.4595 |
| AK3L1 | −0.591 | 0.671 | −0.747 | −0.347 | −0.326 | 0.017 | 0.005 | 0.129 | 0.117 | 1.174 | 0.031 | 0.727 | −0.816 |
| AK5 | 0.123 | −0.335 | −0.52 | 0.111 | −0.201 | −0.277 | −0.444 | −0.038 | 0.004 | 0.449 | −0.763 | 0.23 | −0.019 |
| AKAP1 | 1.3175 | 0.4735 | 0.1445 | 0.5695 | 0.0435 | −8.2625 | 1.5155 | 0.6815 | 0.6425 | 0.5835 | 0.2615 | 1.9485 | 1.5095 |
| AKR1A1 | 0.857 | 0.85 | −0.095 | 0.118 | 0.885 | −0.014 | −0.102 | −0.12 | −0.085 | 0.541 | 0.083 | 0.145 | 0.26 |
| AKR7A3 | 0.3655 | 0.2475 | 0.7545 | −0.0685 | −0.4495 | 0.0595 | 0.1955 | 0.2675 | 0.3765 | 0.7335 | 0.1075 | 1.1545 | −0.0615 |
| ALG5 | −0.2195 | 0.3145 | −0.4015 | −0.5055 | −0.8145 | 1.5765 | 1.6945 | 0.5175 | 0.4845 | 0.0115 | 0.4995 | 0.8305 | 2.4715 |
| ALG8 | 0.0205 | −0.0195 | 0.1315 | 0.0905 | −0.0515 | 0.4195 | −0.3435 | −0.0425 | −0.1395 | −0.4745 | −0.2105 | −0.6545 | −0.5865 |
| AMFR | 0.641 | 0.458 | −0.181 | −0.076 | −0.181 | −0.227 | 0.226 | 0.973 | 1.36 | −0.146 | 0.602 | −0.856 | −0.02 |
| AMPD2 | 1.073 | −1.051 | −0.936 | −0.471 | 0.141 | −0.232 | 0.221 | 0.421 | 1.593 | 0.597 | 0.342 | −0.199 | −0.276 |
| ANAPC11 | −0.141 | 0.306 | −0.013 | −0.273 | −0.451 | −0.113 | −0.174 | −0.056 | 0.056 | 0.243 | 0.584 | −0.635 | −0.343 |
| ANKRD22 | −0.957 | −1.909 | −0.617 | −1.007 | −1.39 | −0.367 | −1.739 | −0.521 | −0.488 | −1.538 | −0.655 | −1.817 | −0.804 |
| ANKRD27 | 0.007 | 0.570 | 0.814 | 0.033 | 0.526 | 0.601 | 0.344 | 0.041 | 0.083 | 0.042 | 0.022 | −0.301 | 0.781 |
| ANLN | −1.03 | −0.088 | −0.581 | 1.045 | −0.398 | −0.709 | −1.958 | −1.528 | −1.57 | −0.656 | −2.537 | −3.327 | 0.072 |
| ANP32A | 1.021 | −0.139 | 0.511 | 0.624 | 1.041 | 0.288 | 1.704 | 0.403 | 0.458 | 0.567 | 0.773 | 0.165 | −0.714 |
| ANXA3 | −2.2555 | −1.5845 | 0.1655 | −0.9455 | −1.5935 | −0.8895 | −2.0265 | 0.7165 | 0.6995 | 0.5285 | −0.3535 | −1.2225 | 0.3615 |
| APBB2 | −1.2895 | −0.8625 | −0.4495 | −0.5385 | −0.9315 | −0.4595 | −1.1675 | −1.2705 | −1.2255 | −0.2525 | −1.3685 | −0.7265 | 0.2485 |
| APOBEC3 | 0.585 | −1.374 | 1.356 | 1.045 | 0.819 | 0.613 | 0.634 | 0.309 | 0.208 | 0.72 | −0.334 | 1.55 | −1.75 |
| AQP5 | 0.471 | 1.522 | 4.039 | 0.957 | 1.816 | 3.969 | −0.61 | 5.308 | 5.326 | −1.133 | 1.663 | −0.26 | 5.13 |
| AR | 0.0765 | −0.3845 | −0.2195 | −0.4775 | 0.0605 | −0.5545 | 0.9045 | 0.4085 | 0.4945 | 0.2685 | −0.4845 | 0.0025 | −0.1715 |
| AREG | −1.903 | 0.414 | 0.19 | 0.32 | 0.96 | 0.203 | −0.915 | −0.072 | 0.011 | 0.471 | −1.514 | 0.768 | −0.151 |
| ARF1 | −0.2805 | 0.2495 | −0.1995 | −0.2505 | 0.0115 | 0.1085 | −0.2825 | 0.4825 | 0.4755 | 0.3995 | 0.2075 | 0.6575 | 0.6935 |
| ARHGDI [symbol] | −0.577 | 0.123 | −0.355 | −0.372 | −0.534 | −0.357 | −0.198 | −0.051 | −0.013 | 0.072 | −0.159 | −0.468 | 0.364 |
| ARL6IP | 1.0245 | 0.3865 | 1.2455 | 0.7575 | 0.3725 | 0.5135 | −0.9695 | 0.4235 | 0.4715 | 0.4235 | 1.0775 | −0.1235 | 0.5415 |
| ARL6IP2 | 1.128 | 0.399 | 0.111 | 0.346 | −0.057 | 0.894 | 1.074 | 1.151 | 1.169 | 1.391 | 1.368 | −0.198 | 0.851 |
| ARMCX2 | 0.922 | 0.083 | −0.038 | 0.503 | 0.081 | 1.049 | 0.785 | 0.92 | 1.037 | 1.336 | 1.232 | 0.085 | 0.75 |
| ARNT2 | 0.108 | 0.454 | 0.327 | −0.066 | −0.455 | −0.041 | −0.435 | 0.873 | 0.887 | 0.081 | 0.206 | −0.359 | −0.86 |
| ARNT2 | 0.3025 | −0.3825 | −1.5405 | 0.3965 | 0.2965 | 0.0325 | −0.9685 | 0.3085 | 0.3135 | −0.0015 | 0.2005 | 0.3315 | −0.4015 |
| ARP3BET [symbol] | −0.9725 | −0.3715 | 0.0555 | −0.1735 | −0.3885 | 0.1125 | −0.3405 | 0.0045 | −0.0255 | −0.1295 | −0.3905 | −0.4775 | 0.0115 |
| ARPC1B | −0.305 | 0.345 | −0.158 | −0.109 | −0.503 | −0.297 | −0.726 | −0.935 | −0.745 | −0.854 | −0.93 | −0.91 | −0.729 |
| ARPC5L | −0.6535 | 0.2105 | −0.6155 | −0.0245 | 0.1435 | −0.3465 | −0.5425 | 0.0175 | −0.0175 | 0.4775 | −0.4475 | −0.3175 | 0.3635 |
| AS89 | 2.559 | 0.274 | −0.235 | −0.078 | 0.399 | −0.404 | −0.447 | −0.111 | −0.077 | 0.801 | 2.42 | −0.385 | 2.06 |
| ASF1A | −0.7025 | 0.0525 | 0.0085 | −0.3675 | −0.2875 | 0.0125 | −1.3835 | 0.1625 | 0.1425 | −0.0775 | −0.2675 | 0.4095 | 0.1185 |
| ASH2L | −1.7 | −2.5 | −0.498 | −1.362 | −1.349 | −0.014 | −2.232 | −0.764 | −0.805 | −0.849 | −1.033 | 0.02 | −1.001 |
| ASK | −1.401 | 0.182 | −0.537 | −0.277 | −0.207 | −0.322 | −0.689 | −0.119 | −0.054 | −0.003 | −0.442 | −0.618 | −0.302 |
| ASPM | −0.9995 | −0.2755 | −0.0235 | −0.9055 | −1.1765 | 0.0145 | −1.1685 | 0.4245 | 0.4345 | −1.0255 | −0.2355 | 0.5376 | −0.1285 |
| ASS | −0.3985 | 0.7865 | −0.5805 | −0.0265 | −0.3855 | 0.1705 | −0.3465 | 0.2965 | 0.3265 | −0.3005 | 0.3245 | 0.4005 | −0.1355 |
| ATAD2 | −0.5415 | 0.5595 | 0.4365 | 0.6455 | 1.2105 | 1.0035 | 1.2695 | 0.6845 | 0.5725 | −0.4365 | −1.6105 | 0.3645 | −0.2855 |
| ATF5 | 0.198 | 0.874 | −0.109 | 0.522 | 0.102 | 0.228 | 0.383 | −0.061 | 0.004 | 0.076 | −0.648 | −0.538 | −0.042 |
| ATF7IP2 | 0.413 | 0.442 | −0.471 | 0.113 | −0.024 | −0.034 | −0.29 | 0.556 | 0.813 | −0.021 | 0.045 | 0.088 | −0.368 |
| ATP2B1 | 0.178 | 0.172 | −0.051 | 0.29 | 0.07 | −0.5 | −0.18 | 0.162 | 0.226 | 0.7 | −0.267 | −0.141 | −0.027 |
| ATP5G1 | 0.1295 | −0.1365 | −0.3805 | −0.1945 | 0.1875 | −0.1795 | 0.8095 | 1.1225 | 1.1415 | 0.4455 | 0.8535 | 0.3995 | 0.4645 |
| ATP5O | −0.972 | 0.347 | −0.933 | −0.789 | −0.746 | −0.008 | −0.982 | 2.813 | 3.001 | −1.544 | −0.801 | 1.248 | −0.032 |
| ATP6AP1 | −0.467 | 0.306 | −0.876 | 0.681 | −0.487 | 0.425 | −0.867 | 0.536 | 0.496 | 0.33 | 0.171 | −0.115 | 0.123 |
| ATP6V0A4 | −0.17 | 0.634 | 0.733 | 0.152 | −0.25 | 0.145 | 0.823 | 0.62 | 0.685 | 0.524 | 0.356 | −0.733 | 0.834 |
| ATP6V1B [symbol] | 1.0265 | 0.3225 | 0.5385 | −0.1925 | 0.4355 | −0.2285 | −0.0225 | 0.3095 | 0.2915 | 0.2025 | 0.8865 | −0.5345 | 0.2385 |
| ATP6V1G [symbol] | −0.181 | 0.087 | 0.684 | 0.462 | 0.38 | 0.462 | 0.687 | 0.7 | 0.699 | 0.635 | 0.155 | −0.589 | 0.68 |
| ATPIF1 | −0.2455 | 0.0075 | 0.3905 | −0.1735 | −0.1275 | 0.2065 | −0.7205 | −0.7245 | −0.7505 | −1.0825 | −1.3725 | −0.9065 | −0.9385 |
| ATXN10 | −0.288 | 0.638 | −0.063 | 0.581 | 0.748 | −0.127 | −0.812 | −0.49 | −0.43 | −1.037 | −0.529 | −1.348 | −0.506 |
| B3GNT5 | 0.0645 | 0.1395 | −0.0545 | 0.4445 | 0.6735 | 0.2585 | 2.7165 | 0.5495 | 0.4965 | 0.2245 | 0.5865 | 0.3935 | 3.0025 |
| BACE2 | 2.2625 | 0.5155 | −0.0365 | −0.0205 | 0.4315 | −0.2235 | 0.0295 | −0.0705 | −8.1085 | 0.2385 | 1.1905 | 0.0005 | 1.9645 |
| BAG1 | 0.8535 | −0.4815 | 0.2575 | 1.2555 | 1.2215 | −0.7555 | 2.0955 | 0.8145 | 0.6405 | 0.7695 | 0.4355 | 0.4525 | −0.0915 |
| BAG4 | −0.3885 | 2.5745 | 2.2365 | −1.3055 | 0.9895 | 0.5095 | 1.0675 | −1.4825 | −1.5125 | −0.5315 | −1.3665 | −1.1765 | 1.3885 |
| BAI2 | 1.225 | 1.224 | 0.334 | −0.284 | −0.114 | −0.377 | 2.244 | 8.036 | 0.011 | 1.831 | 1.868 | −1.001 | −0.574 |
| BAMBI | −0.641 | −0.224 | 0.06 | −0.306 | −0.448 | −0.106 | −0.256 | 0.365 | 0.437 | −1.413 | −0.329 | 0.482 | −0.337 |
| BCAS1 | −0.176 | −1.282 | −0.052 | 0.092 | 0.971 | 1.45 | 1.832 | 2.478 | 2.449 | 2.607 | 1.655 | 3.435 | 3.293 |
| BCL2L14 | 1.0565 | 0.0275 | 8.0005 | −0.7845 | 1.6915 | 5.7195 | −1.7485 | 5.8515 | 5.7665 | 4.8945 | −0.2945 | −1.4785 | 0.0485 |
| BCMP11 | −0.512 | −0.065 | 3.308 | −2.116 | 0.162 | 3.053 | −1.128 | 3.155 | 3.118 | 2.472 | 0.135 | −2.056 | −0.86 |
| BEX1 | −0.9265 | 0.9865 | −1.1525 | 1.2635 | 2.7195 | 0.8225 | 3.9235 | 0.6425 | 0.7465 | 2.1205 | −3.5565 | 1.7745 | −0.6215 |
| BEX2 | 8.1485 | −0.5425 | −0.1455 | −0.2875 | −0.5485 | −0.3085 | −0.2495 | −1.0165 | −0.8705 | −0.0265 | 0.8385 | −2.0705 | −0.1695 |
| BF | −0.107 | 0.286 | 0.12 | 0.029 | 0.031 | −0.709 | 0.684 | 0.544 | 0.054 | 1.113 | 0.6 | 0.237 | 0.759 |
| BFSP1 | 0.462 | 0.603 | 0.157 | −0.003 | 0.121 | −0.16 | −1.034 | −0.128 | −0.197 | 0.093 | −0.011 | −0.839 | −0.27 |
| BIK | −1.335 | −2.938 | −1.079 | −1.483 | −1.108 | −0.527 | −2.864 | 0.106 | 0.074 | −0.552 | −0.175 | −0.516 | −0.963 |
| BIRC2 | 0.3915 | 0.2015 | 0.0285 | −0.0405 | −0.0345 | −0.0585 | 0.1275 | 0.3285 | 0.3715 | 1.8355 | 0.4175 | −0.1155 | −0.5585 |
| BIRC5 | 1.1655 | 0.5195 | 0.1765 | 0.7835 | 1.1785 | 0.0385 | 0.7235 | −0.7525 | −0.5735 | −0.2075 | −0.0855 | −0.2255 | −0.4795 |
| Bit1 | 0.6135 | −0.0835 | 0.2235 | −0.1315 | −0.2805 | 0.0355 | 0.7575 | 0.9905 | 0.9315 | 0.1765 | 0.7785 | 1.7365 | 0.3645 |
| BIVM | −0.382 | −1.019 | −0.809 | −0.531 | −0.351 | −0.488 | −1.185 | −0.432 | −0.241 | −0.085 | −0.316 | −1.016 | −0.793 |
| BLVRA | 0.305 | −0.251 | −0.703 | 1.803 | 1.235 | 0.026 | −0.155 | 0.798 | 0.869 | −0.406 | −0.187 | 0.789 | −0.487 |
| BM039 | 0.159 | 0.712 | 0.259 | 0.391 | 0.247 | 0.317 | −0.11 | −0.395 | −0.502 | −0.83 | −0.448 | 0.158 | −0.74 |
| BMP7 | 0.548 | −0.274 | −0.317 | −0.274 | 0.036 | 0.358 | −0.762 | 0.536 | 0.516 | −0.914 | 0.616 | 0.408 | 0.198 |
| BOC | 2.6345 | 0.2955 | 0.2925 | −0.0915 | 0.4015 | −0.3705 | −0.5435 | 0.9245 | 0.9445 | 1.3235 | 3.1135 | −1.5145 | 3.0625 |
| BRCA1 | 0.6955 | −0.0565 | 0.0005 | 0.1315 | 0.1595 | −0.4125 | −0.0655 | 0.2015 | 0.2915 | 0.2475 | 0.0365 | −0.5555 | −0.5635 |
| BRF2 | 0.218 | −0.049 | −0.272 | 0.027 | −0.04 | 0.059 | 0.124 | −0.043 | −0.01 | −0.527 | −1.061 | −0.184 | −0.667 |
| BRI3 | 0.281 | 1.077 | 0.206 | 0.01 | 1.73 | 0.496 | −0.01 | 1.202 | 1.185 | 0.142 | 0.353 | 0.558 | 0.448 |
| BRI3BP | 0.84 | 0.313 | 0.501 | 0.284 | 0.541 | 0.309 | 0.808 | −0.43 | −0.43 | 0.691 | 0.029 | −0.12 | −0.011 |
| BSPRY | 0.3635 | 0.4405 | −0.0495 | −0.3955 | −1.1035 | 0.2085 | −1.3385 | −0.3565 | −0.3075 | −0.3655 | −0.4555 | −2.2245 | −0.5965 |
| BTF3 | 0.1255 | 0.0905 | −0.5975 | −0.4785 | −0.3495 | −0.5595 | −0.3985 | 0.3665 | 0.4135 | −0.0525 | 0.2025 | 0.2245 | −0.1795 |
| BTG3 | −0.914 | −0.951 | −0.403 | −1.028 | −1.338 | −0.27 | −1.521 | −0.299 | −0.424 | −0.474 | 0.118 | −1.201 | −0.283 |
| BγN2A2 | 0.447 | −0.88 | 0.421 | −0.321 | −0.695 | −0.224 | 0.488 | 0.985 | 0.899 | 0.194 | −0.774 | 0.426 | 1.004 |
| BUB1 | −0.0445 | −0.0785 | −0.0265 | 0.1075 | 0.4295 | 0.0155 | −0.4435 | −0.6965 | −0.6895 | −0.0775 | −0.7325 | −0.4055 | −0.2445 |
| BUB3 | −0.084 | −0.33 | 0.183 | 0.114 | 0.286 | −0.058 | 0.076 | −0.661 | −0.808 | −0.008 | 0.333 | 0.496 | 0.137 |
| BXDC1 | 0.227 | 0.232 | 0.131 | −0.444 | −0.689 | −0.18 | −0.058 | 0.103 | 0.15 | 0.056 | 0.928 | 0.868 | 0.738 |
| BZRP | 0.759 | 2.465 | −0.345 | 0.276 | −0.087 | −0.034 | −0.413 | 0.603 | 0.744 | 0.169 | −1.176 | 1.383 | 0.96 |
| BZW1 | −0.5405 | 0.2925 | −0.2445 | 0.1375 | −0.1795 | −0.3595 | 0.1545 | −0.7945 | −0.5255 | −0.2435 | −0.0405 | −0.2525 | −0.6045 |
| C10orf116 | 0.2295 | 0.1895 | 0.0735 | 0.3655 | 0.4985 | 0.1095 | −0.3495 | 0.4145 | 0.4225 | 0.4295 | 0.0145 | −0.7145 | 0.8065 |
| C10orf38 | 0.596 | −0.425 | −0.608 | 1.188 | 0.34 | −0.392 | −0.571 | −0.214 | −0.205 | 0.584 | −1.104 | 1.34 | 0.435 |
| C10orf4 | 0.1605 | −0.2665 | −0.4075 | −0.2015 | −0.4365 | −0.1785 | −0.4315 | −0.0625 | 0.0095 | −0.1815 | 0.1825 | −0.0575 | 0.0045 |
| C10orf45 | −0.3705 | 0.3755 | 0.7635 | 0.2065 | 0.1085 | 0.7105 | 1.0175 | 1.5235 | 1.4115 | 2.8495 | −0.5885 | 1.9505 | 3.2175 |
| C10orf7 | 0.3215 | −0.3775 | −0.1715 | 0.2625 | 0.0325 | −0.0765 | −0.9325 | −0.7405 | −0.8905 | −0.3775 | −0.3855 | 0.2885 | 0.8995 |
| C10orf82 | 0.137 | −0.613 | −0.599 | −0.398 | −0.093 | 0.389 | −0.191 | 0.017 | −0.1 | −0.884 | −0.519 | −0.915 | −0.354 |
| C11orf24 | 0.9755 | −0.7365 | 0.3225 | 0.6895 | 0.8495 | 0.9305 | −0.4875 | 1.7065 | 1.6175 | −0.0495 | 0.4885 | −1.2195 | 0.4855 |
| C12orf14 | 0.388 | 0.318 | −0.647 | 0.068 | 0.039 | 0.087 | 0.249 | −0.594 | −0.553 | −0.396 | 0.139 | −0.631 | 0.135 |
| C13orf21 | −0.496 | −0.179 | 0.074 | −0.409 | −0.187 | −0.487 | 0.349 | 0.082 | −0.021 | −0.305 | −0.172 | −0.352 | −0.407 |
| C13orf23 | −0.026 | −0.996 | −0.584 | −0.744 | −0.791 | −0.504 | −0.825 | −0.037 | −0.074 | −0.206 | −0.489 | 0.23 | 0.182 |
| C14orf94 | 0.48 | 1.334 | 0.31 | 0.644 | 1.908 | 1.724 | 1.99 | −0.104 | −0.104 | 1.47 | 0.443 | −0.72 | 1.17 |
| C15orf23 | 0.2335 | −0.0545 | −0.0795 | −0.3745 | −0.4485 | −0.2165 | 0.4965 | 1.3735 | 1.1555 | −0.2365 | −0.1865 | −0.2115 | 0.3655 |
| C16orf45 | 0.542 | −0.191 | 1.167 | −0.271 | 0.65 | 0.772 | −0.24 | 0.138 | 0.194 | 1.125 | 1.28 | 0.717 | −0.918 |
| C17orf37 | 0.133 | −0.375 | −0.15 | 0.197 | 0.228 | 0.081 | −0.047 | 0.325 | 0.281 | 0.356 | −0.213 | −0.404 | 0.28 |
| C18orf1 | 0.663 | −0.437 | 0.33 | 0.662 | 0.624 | 0.226 | 0.184 | −1.506 | −1.394 | 0.977 | −0.579 | 0.755 | −0.16 |
| C19orf10 | −0.8625 | 0.6265 | 0.4665 | −0.5675 | 0.5405 | 1.5345 | 1.4895 | 1.7545 | 1.6555 | 1.4585 | 0.3495 | 1.6545 | 2.0785 |
| C19orf32 | 2.0925 | 1.2115 | 2.0705 | −0.5055 | −1.1015 | −0.5185 | −1.6765 | −0.4645 | −0.4815 | −0.4305 | 0.3045 | 0.7375 | 0.8405 |
| C19orf33 | 1.533 | 2.016 | 2.006 | −0.387 | 0.683 | 0.393 | 1.374 | 1.706 | 1.697 | 0.794 | 1.228 | 0.952 | 1.781 |
| C1orf24 | 1.885 | 1.482 | 1.776 | 1.242 | 1.312 | 1.518 | 1.202 | 1.628 | 1.961 | 0.934 | 1.185 | −0.358 | −0.539 |
| C1orf34 | −0.1865 | −0.4855 | −0.0105 | −0.1655 | −0.0255 | −0.1435 | 1.2475 | 0.2855 | 0.3105 | −0.2305 | 0.0685 | −0.8675 | 0.1075 |
| C20orf103 | 1.8235 | −1.4785 | 0.8585 | 0.6735 | 0.5215 | 0.4735 | −0.4475 | 0.6735 | 0.8165 | 1.1055 | 0.3475 | −0.1655 | 0.8555 |
| C20orf149 | −0.105 | 0.583 | 0.568 | −0.536 | 0.25 | −0.418 | 0.778 | −0.531 | −0.512 | 0.645 | −0.179 | 0.019 | −0.218 |
| C20orf23 | −0.610 | 0.128 | −0.409 | 0.634 | 0.456 | 0.419 | 1.039 | 0.177 | 0.139 | 0.538 | 0.35 | 1.381 | 0.593 |
| C20orf3 | −0.168 | −0.208 | −0.167 | −0.492 | −0.175 | −0.27 | 1.066 | 0.44 | 0.398 | −0.193 | 0.246 | −0.096 | −0.355 |
| C20orf35 | 0.611 | 0.586 | 0.447 | 0.362 | 0.364 | 0.475 | 0.278 | 0.434 | 0.487 | 0.2 | −0.058 | −1.284 | −0.428 |
| C20orf43 | −0.5335 | −0.7305 | −0.0415 | −0.1875 | −0.1815 | −0.3995 | −0.8015 | −0.8355 | −0.4145 | −0.3295 | −0.3975 | −0.9155 | −0.3815 |
| C20orf8 | −0.858 | −1.101 | −0.58 | −0.48 | −0.765 | −0.557 | −1.252 | −0.264 | −0.196 | −0.336 | −0.414 | 0.578 | 0.128 |
| C21orf45 | −0.554 | 0.029 | −0.255 | −0.148 | −0.001 | −0.316 | 0.001 | 0.069 | 0.197 | −0.037 | 0.231 | −0.984 | −0.306 |
| C22orf18 | 0.912 | 0.73 | 0.541 | −0.097 | 0.276 | 0.008 | 0.296 | 0.98 | 0.888 | 0.551 | −0.072 | −0.018 | 0.014 |
| C2F | −0.97 | 0.781 | −0.508 | 1.558 | 2.138 | 0.183 | −0.546 | 0.442 | 0.228 | 1.166 | −0.95 | 1.338 | −0.024 |
| C2orf17 | 1.071 | −0.317 | 0.16 | 1.127 | 0.074 | 0.684 | −1.186 | 0.935 | 0.849 | 0.603 | 0.345 | −0.013 | −1.41 |
| C4A | 0.495 | −0.791 | 0.192 | −0.187 | 0.348 | −0.19 | 0.985 | 0.6 | 0.669 | 0.011 | 0.427 | 0.281 | −0.184 |
| C5orf13 | −0.624 | 0.136 | −0.533 | 0.008 | 0.074 | 0.205 | −0.297 | −0.104 | −0.133 | −0.107 | 0.108 | 0.265 | 0.341 |
| C6orf108 | −0.387 | 0.08 | −0.453 | 0.002 | −0.332 | 0.483 | −1.16 | −0.837 | −0.802 | −0.318 | −0.548 | −0.566 | 0.348 |
| C6orf166 | 0.072 | 0.845 | 0.801 | 2.111 | 1.055 | 0.444 | 0.891 | 2.036 | 2.014 | 1.116 | 3.798 | 1.103 | 1.8 |
| C6orf192 | −0.3745 | 0.4695 | −0.3355 | −0.1815 | 0.2475 | −0.2915 | 0.3005 | 0.4075 | 0.4035 | 0.2565 | −0.3685 | 0.0355 | 0.1685 |
| C6orf211 | −0.831 | 1.132 | 1.045 | −0.006 | 1.161 | 0.325 | 0.101 | −0.968 | −0.928 | 0.334 | −1.584 | 0.49 | −0.204 |
| C6orf49 | −1.0785 | 0.0645 | −0.5455 | −0.1585 | −0.6775 | −0.3025 | −0.7385 | 0.9255 | 0.9135 | 0.5265 | −0.3675 | 0.3575 | −0.7745 |
| C6orf85 | −0.2375 | 0.3725 | 0.2585 | 0.7505 | 0.1165 | 0.5805 | 0.4425 | 0.0615 | 0.0045 | 0.1735 | −0.4755 | 1.0165 | −0.2085 |
| C7orf24 | 1.073 | 3.131 | 2.792 | 1.132 | 0.968 | 3.113 | 2.1 | 0.595 | 0.675 | 1.639 | 0.6 | 2.321 | 0.99 |
| C9orf10 | −2.242 | −0.12 | −0.505 | −1.527 | −1.784 | −1.522 | −1.063 | −0.538 | −0.521 | −0.507 | −1.031 | −0.687 | −1.137 |
| C9orf121 | −0.21 | −0.239 | −0.546 | 0.188 | 0.235 | −0.221 | −0.264 | 0.001 | 0.007 | −0.377 | −0.125 | 0.477 | −0.056 |
| C9orf58 | −0.2625 | 0.0965 | −0.3065 | −0.0055 | 0.3325 | 0.2265 | −0.5245 | −0.0935 | −0.0275 | 0.0205 | 0.4775 | 0.0405 | 0.2655 |
| C9orf88 | 0.691 | 1.337 | −0.142 | 0.378 | 1.057 | 1.183 | 2.084 | 0.341 | 0.506 | 0.671 | −0.289 | 1.969 | 0.911 |
| C9orf9 | −2.016 | 1.351 | −0.044 | −0.304 | −0.813 | 1.541 | −0.09 | 0.039 | 0.128 | −1.641 | 0.168 | 3.179 | 1.403 |
| CA12 | −2.109 | 1.518 | −0.128 | −0.227 | −0.757 | 1.719 | −0.24 | 0.321 | 0.196 | −0.339 | 0.083 | 3.225 | 1.429 |
| CA8 | −0.003 | 0.24 | −0.006 | 0.439 | 0.155 | 9.151 | −0.087 | 0.336 | 0.187 | 0.178 | −0.204 | 0.046 | 0.513 |
| CA8 | 0.0395 | 0.2245 | −0.3835 | 0.2305 | −0.0575 | −0.0395 | 0.0185 | 0.0375 | −0.2905 | 0.2225 | −0.2455 | 0.0195 | −0.1445 |
| CACNA1G | −0.541 | 0.161 | −0.538 | −0.374 | −0.729 | −0.435 | −0.549 | −0.505 | −0.396 | −0.702 | −0.48 | 0.292 | −0.733 |
| CALCA | −0.881 | −0.739 | −0.313 | −0.902 | 0.939 | −0.635 | −1.32 | −0.678 | −0.878 | 0.117 | −0.48 | 5.104 | −0.811 |
| CALML4 | 0.9265 | 1.2625 | 1.4935 | 0.7365 | 0.4345 | 1.4235 | 0.8515 | 0.9985 | 1.0295 | 0.3125 | 0.7835 | 0.3995 | 0.7805 |
| CALML5 | 0.8025 | 1.9185 | 1.4195 | 0.8705 | 0.3745 | 0.6175 | −2.2615 | −0.5035 | −0.4045 | 0.0235 | −1.2815 | 0.9325 | −0.1015 |
| CaMKIINa [symbol] | 0.053 | −0.333 | 0.4 | 0.422 | 0.416 | 0.538 | 0.212 | −0.18 | −0.174 | 0.233 | 1.002 | 0.097 | 0.142 |
| CAP2 | 0.846 | −0.327 | −0.558 | −0.347 | 0.633 | 0.856 | −0.443 | 2.217 | 1.112 | 0.774 | 0.705 | 1.64 | 0.281 |
| CAP350 | −1.3305 | 0.0535 | 0.9025 | −0.1145 | −0.8225 | 1.0295 | −0.5985 | 0.4025 | 0.4325 | −1.6135 | −0.2385 | −1.5135 | 0.3085 |
| CAPN13 | 0.303 | 0.599 | 0.304 | 0.121 | 0.911 | −0.038 | 0.101 | 1.433 | 1.36 | −0.107 | 0.974 | −0.098 | 0.157 |
| CAPN9 | 0.0275 | 0.6045 | 0.0725 | 0.0525 | 0.1105 | 0.0015 | 0.3235 | 0.0185 | −0.0355 | 0.3285 | −0.2645 | −0.4465 | 3.1405 |
| CARD14 | −0.4875 | −0.3855 | −0.5565 | 0.0415 | −0.2075 | 0.3105 | −0.8325 | 0.8385 | 0.7305 | 1.1075 | −1.1375 | −0.5105 | −0.3065 |
| CART | 0.1575 | −0.6435 | −0.4765 | −0.4065 | 0.9715 | −0.0995 | −0.2945 | 0.0475 | 0.0885 | −0.0575 | 0.0815 | −1.4615 | −0.2815 |
| CASP1 | 0.909 | −0.361 | −0.164 | 0.186 | −0.076 | 0.538 | 1.569 | 0.655 | 0.812 | 0.94 | −0.862 | −0.152 | 0.309 |
| CASP3 | 0.136 | 0.12 | 0.506 | 0.422 | −0.324 | 0.201 | 0.122 | 0.057 | 0.621 | 0.826 | 0.153 | 0.201 | 0.111 |
| CASP7 | 0.4145 | 0.8595 | −0.7255 | 0.3285 | 0.1875 | −0.0875 | −0.7455 | 0.1825 | 0.3265 | −0.1205 | −0.6235 | −0.5255 | −0.2655 |
| C9LN1 | −1.426 | 0.318 | −1.092 | −0.387 | −1.008 | −0.466 | −2.214 | −0.174 | −0.128 | −0.363 | −0.669 | 0.08 | −0.509 |
| CBR1 | 0.6295 | 0.3205 | 0.4725 | 0.2645 | 0.1835 | 0.0005 | −0.0735 | 0.5965 | 0.5385 | 0.5695 | 0.0115 | −0.3905 | 0.1055 |
| CBR3 | −0.713 | −0.321 | −0.178 | 0.105 | −0.002 | 0.002 | 0.035 | −0.258 | −0.313 | −0.141 | 0.085 | 0.198 | 0.109 |
| CBR4 | −0.3295 | −0.1315 | 0.0085 | −1.4455 | 0.3335 | −0.3635 | 3.9815 | −1.5445 | −1.8805 | 0.0425 | −0.6395 | 1.5395 | −0.3555 |
| CCL24 | −0.995 | −2.826 | −0.421 | −1.037 | −0.723 | −0.476 | −1.58 | −0.351 | −0.349 | −0.677 | −0.354 | −0.397 | −0.769 |
| CCL29 | 1.554 | 0.142 | −0.625 | 0.26 | 0.054 | 0.583 | 0.979 | 0.246 | 0.243 | 0.552 | 1.196 | 0.452 | 2.717 |
| CCNB2 | −0.1055 | −0.8125 | −0.2185 | −0.5165 | −0.4095 | −0.3515 | −0.7405 | −0.3345 | −0.2255 | −0.7765 | 0.4415 | −0.0635 | −0.5425 |
| CCND1 | 0.779 | 1.329 | 1.72 | 0.321 | 0.872 | 1.136 | 0.904 | 0.917 | 1.021 | 1.27 | −0.836 | 0.821 | 1.758 |
| CCNE1 | 0.4455 | 0.3095 | 0.2295 | −0.0475 | 6.3655 | −0.1035 | 0.4505 | −0.3195 | −0.2515 | 0.6815 | −0.1665 | −0.7975 | 0.5115 |
| CCNG2 | −1.644 | −0.545 | 0.551 | −0.861 | −1.489 | 0.076 | −1.056 | 0.52 | 0.501 | −0.616 | −0.996 | 0.488 | 0.598 |
| CCNH | −1.0035 | −0.6885 | −0.6715 | −0.1115 | −0.2255 | −0.0635 | −0.5285 | 1.1935 | 1.2475 | 0.2455 | −0.2785 | 0.5395 | −0.1265 |
| CD38 | −0.7355 | −0.3585 | −0.5595 | 0.1595 | 0.1845 | −0.0785 | −0.3595 | −0.4385 | −0.4195 | −0.6105 | −0.2515 | 0.6945 | 0.5685 |
| C030 | 0.2095 | 0.2235 | 0.4855 | 0.3185 | −0.6125 | 0.5465 | −0.5535 | 0.1355 | 0.1485 | 0.3255 | −0.5655 | −0.7445 | −0.2665 |
| CD4 | 0.3155 | −0.5535 | −0.3755 | 0.8595 | 0.6345 | 0.3215 | 0.5775 | −0.0895 | −0.0735 | 0.0325 | −0.6505 | 0.3765 | 0.2195 |
| CD58 | −0.653 | −2.332 | −0.383 | −0.402 | −1.276 | −0.328 | −1.854 | −0.164 | 0.024 | −0.287 | −0.409 | −0.233 | −0.082 |
| CD50 | −0.1565 | −1.2675 | −0.6465 | −0.6605 | −0.5515 | −0.3215 | −1.2135 | 0.2235 | 0.2745 | −0.9145 | −0.5785 | −0.0125 | −0.6215 |
| CDC2 | −1.4215 | −1.1555 | −1.2815 | −1.4075 | −1.1105 | −0.3555 | −2.2675 | −0.6045 | −0.5125 | −0.6685 | 0.9625 | 0.0845 | −0.4015 |
| CDC6 | −0.9965 | −1.7595 | −0.8875 | −1.8885 | −1.3195 | −0.7135 | −2.3565 | −0.4015 | −0.3465 | −1.3785 | −0.5015 | −0.8455 | −1.3615 |
| CDCA1 | −1.276 | −1.858 | −0.478 | −0.713 | −1.097 | −0.718 | −1.665 | 0.339 | 0.367 | −1.903 | −0.267 | −0.816 | −0.805 |
| CDCA5 | 0.997 | 0.338 | 0.582 | −0.454 | −0.539 | −0.739 | 1.028 | −0.732 | −0.828 | 0.728 | 1.019 | −0.141 | 0.736 |
| CDCA7 | 0.0345 | −1.7395 | −1.9865 | −0.9835 | −0.9625 | 0.5955 | −0.0705 | −0.3595 | −0.3605 | −0.7335 | −1.1365 | −1.3455 | 0.8085 |
| CDH1 | 0.052 | −0.027 | −0.398 | −0.181 | −0.618 | 0.225 | −0.638 | −0.685 | −0.685 | −0.66 | −0.868 | −0.11 | −0.117 |
| CDH3 | −0.8705 | 0.4375 | −0.3275 | −0.1415 | 0.3075 | −0.1105 | 0.1055 | −0.3915 | −0.3885 | −0.1615 | 0.3215 | 0.2805 | −0.4945 |
| CDK2 | −0.0635 | 0.4075 | −0.7095 | −0.4725 | −0.5135 | 0.0775 | −0.7035 | 0.3955 | 0.4245 | −0.1095 | −0.1625 | 0.3395 | 0.1015 |
| CDKN2A | −0.5205 | −1.6705 | −0.6025 | −0.5915 | −0.8595 | −0.3865 | −2.3365 | −0.0295 | 0.0495 | 0.3675 | −0.7665 | −0.2615 | −0.4526 |
| CDKN2D | −0.207 | −2.251 | −1.249 | −0.731 | −0.819 | −0.777 | −1.484 | −0.241 | −0.289 | 0.229 | 0.224 | −0.475 | −1.009 |
| CDKN3 | −0.005 | 0.13 | −0.082 | −0.059 | −0.448 | −0.165 | 0.313 | 0.229 | 0.076 | 0.081 | 0.377 | 0.082 | −0.355 |
| CDT1 | 0.0185 | 1.6735 | 0.8755 | 0.9295 | 1.4345 | 1.0475 | 1.5645 | 1.1055 | 1.2195 | 1.0205 | 0.7665 | 2.0725 | 1.9285 |
| CE9PG | −0.576 | −2.041 | −1.013 | −0.81 | −0.712 | −0.551 | −1.201 | −0.595 | −0.516 | −0.456 | −0.11 | −0.599 | −0.39 |
| CELSR1 | −0.62 | −0.336 | −0.024 | −0.836 | −0.526 | −0.058 | −0.46 | −0.856 | −0.546 | −0.706 | 0.274 | −0.046 | −0.484 |
| CENPA | −1.618 | −3.121 | −0.556 | −1.546 | −0.533 | 0.056 | −2.678 | −1.26 | −1.325 | −0.874 | −1.355 | −0.7 | −0.852 |
| CENPE | −0.137 | 0.289 | 0.149 | −0.064 | −0.302 | −0.208 | −0.14 | 0.94 | 0.793 | −0.543 | −0.174 | −0.433 | 0.323 |
| CENPF | 9.3675 | −0.3885 | −0.2885 | −0.0035 | −0.2195 | −0.0195 | 0.0605 | −0.4565 | −0.4155 | −0.5235 | 0.1875 | 0.1875 | −0.0475 |
| CENTG3 | 1.2975 | 0.1145 | 0.6465 | −0.2265 | −0.2425 | −0.1155 | 0.1705 | 1.0095 | 0.9155 | 0.9125 | 0.6295 | 0.0815 | 0.3805 |
| CES2 | −0.991 | 1.954 | −0.534 | 0.055 | 3.873 | −0.137 | 0.506 | 4.572 | 4.465 | 3.19 | 1.564 | −0.267 | 4.42 |
| CETN3 | −0.8855 | 0.4475 | −0.1765 | −0.6325 | −0.0405 | 0.1305 | 0.5065 | 1.1195 | 1.1155 | 0.3265 | 0.1495 | −0.5855 | 0.2955 |
| CGA | 0.2465 | 1.0025 | −0.1535 | −0.0765 | −0.3135 | −0.0685 | −0.3465 | 1.4165 | 1.3485 | 0.3625 | 0.4135 | −0.6585 | 0.0765 |
| CGI-12 | 0.5315 | 0.6405 | −0.3775 | −0.0455 | 0.9855 | 1.3815 | 2.3255 | 0.0105 | −0.0545 | 1.8765 | 0.7405 | 0.2875 | −0.2805 |
| CGI-49 | −0.215 | 0.316 | 0.576 | −0.053 | 0.325 | −0.162 | 1.434 | 1.143 | 1.1 | 0.691 | 1.585 | 0.077 | 2.526 |
| CGI-4 [symbol] | 0.187 | −0.665 | 2.256 | 1.166 | −0.965 | −0.389 | −2.182 | 1.004 | 1.016 | 0.568 | −1.778 | −0.679 | 1.024 |
| CGN | −0.71 | −0.437 | −0.71 | −0.367 | −0.597 | −0.487 | −1.982 | −0.387 | −0.317 | 0.073 | −0.007 | 0.071 | −0.61 |
| CGNL1 | 0.1655 | −0.0105 | −1.0885 | −0.1765 | −0.2715 | −0.0455 | −0.2295 | −0.5445 | −0.5155 | −0.2125 | −0.6245 | 0.0185 | 0.1095 |
| CHAF1B | 0 | 0.223 | 0.058 | −0.462 | 0.108 | −0.344 | 0.736 | −0.563 | −0.544 | −0.023 | 0.107 | −0.42 | 0.478 |
| CHC1 | 0.591 | 0 | 0.333 | 0.176 | 0.329 | 0.279 | 0.876 | 0.478 | 0.404 | 0.656 | 0.122 | 1.074 | 0.879 |
| CHCHD2 | −1.474 | −0.357 | −1.312 | −0.772 | −0.06 | 1.129 | −0.767 | 1.657 | 1.643 | −0.119 | −1.212 | 1.604 | 0.627 |
| CHCH05 | −0.747 | −0.301 | −1.552 | −0.758 | −0.871 | 0.173 | 0.108 | 0.935 | 0.93 | 0.322 | −1.68 | −1.171 | −0.341 |
| CHI3L1 | −0.499 | 0.174 | −0.526 | −0.546 | −0.262 | −0.321 | −0.035 | −1.021 | −1.022 | −0.596 | −0.689 | 0.199 | 1.082 |
| CHI3L2 | −0.387 | −0.217 | −0.244 | 0.091 | −0.219 | −0.089 | 0.009 | −0.292 | −0.238 | −0.357 | 0.086 | 0.052 | −0.223 |
| CHKA | −9.463 | 0.083 | −0.125 | −0.116 | 0.026 | −0.19 | −0.261 | 0.839 | 0.559 | −0.228 | −0.117 | −0.023 | 0.107 |
| CHCDL | −0.2855 | −0.1495 | −0.1365 | 0.5115 | −0.0065 | 0.1955 | 0.1115 | −0.7045 | −0.6545 | −0.7455 | −0.0745 | 0.0435 | −0.1275 |
| CHRDL2 | 0.203 | 0.499 | 0.665 | 0.352 | −0.112 | 0.355 | 0.434 | −0.355 | −0.444 | 0.46 | −0.416 | −0.171 | 0.407 |
| CHST2 | 0.3875 | 0.2485 | 0.2625 | −0.4595 | −0.1395 | −0.0735 | −0.5345 | 0.4585 | 0.3885 | 0.1895 | 0.0285 | 0.4075 | 0.2225 |
| CHURC1 | −0.118 | −0.322 | 0.276 | −0.017 | 0.452 | −0.037 | 0.133 | −0.048 | 0.039 | 0.774 | −0.5 | −0.492 | −0.002 |
| CIR | 0.7675 | −0.1825 | −0.4385 | 0.6485 | −0.0455 | 0.0395 | 0.3035 | 0.3115 | 0.3965 | 0.0175 | 0.2715 | −0.3275 | −0.4565 |
| CIRBP | 0.8925 | 0.4925 | −0.0555 | 0.1705 | −0.1825 | 0.0945 | −0.0525 | 0.6305 | 0.5865 | −0.3855 | 0.4465 | −1.9655 | −0.4785 |
| CKLF | 0.1905 | −0.3745 | 0.2855 | −0.5135 | −0.3415 | 0.1945 | −0.7475 | −0.5605 | −0.5035 | −1.1365 | −1.0365 | −0.1995 | −0.5195 |
| CKLFSF6 | −0.0555 | 0.0855 | −1.1305 | −1.3815 | −1.4755 | 0.1995 | 0.0955 | 1.1645 | 0.8125 | −0.3485 | −0.0475 | −0.8965 | −1.9395 |
| CKLFSF7 | −0.747 | −0.967 | 0.091 | −0.621 | −0.083 | −0.087 | −0.982 | −0.549 | −0.613 | −0.542 | −0.48 | 0.46 | 0.773 |
| CKMT1 | −0.804 | 0.034 | 0.086 | −0.284 | −0.972 | 0.305 | −1.05 | −0.174 | −0.128 | 0.138 | −0.511 | 0.613 | −0.714 |
| CKS1B | −0.653 | −0.236 | −0.07 | 0.008 | −0.793 | 0.052 | 0.412 | 0.737 | 0.693 | 0.636 | 0.092 | −0.051 | 0.036 |
| CKS2 | 0.8775 | −0.6985 | −0.6355 | −0.3965 | −0.1735 | −0.4105 | 0.0005 | −0.1125 | 0.2525 | 0.0115 | 0.6215 | −2.0975 | −0.0095 |
| CL640 | 0.4665 | 0.4005 | −0.0305 | −0.4875 | 0.5695 | −1.1385 | 1.3915 | 0.8515 | 0.8185 | −0.1225 | −1.0215 | −0.5835 | 0.0645 |
| CLDN23 | −0.7335 | −0.3615 | −1.0105 | −1.5945 | −0.3165 | −0.4315 | 0.0595 | 0.2915 | 0.2525 | −0.1625 | −0.8105 | 0.9035 | 0.4065 |
| CLDN3 | 1.5615 | 0.5245 | −0.1725 | −1.0275 | −0.5475 | −0.3885 | 0.0195 | 1.0685 | 1.1375 | −0.6115 | −0.3635 | 0.8225 | −0.3875 |
| CLDN4 | 1.13 | 2.477 | 1.894 | 2.048 | 1.255 | −0.262 | −2.37 | 0.276 | 0.203 | 2.474 | −0.791 | 4.231 | 0.215 |
| CLDN8 | 3.3195 | 0.8955 | −0.2835 | 1.2695 | 2.0505 | 2.5935 | −0.2575 | 3.6795 | 3.4135 | 0.0555 | 1.7255 | −0.3325 | 3.3835 |
| CLGN | −0.4435 | 0.0965 | 0.0115 | −0.2915 | −0.1965 | −0.2875 | −0.3275 | 0.2455 | 0.2215 | 2.3025 | −0.2855 | 0.3225 | 0.0625 |
| CLIC6 | 0.613 | 0.099 | 0.404 | 0.417 | 0.261 | 0.092 | 0.322 | 1.13 | 0.95 | 1.71 | 1.103 | −0.56 | 0.309 |
| CLNS1A | 0.2175 | −0.3575 | −1.6475 | 1.2285 | 1.2725 | −0.8885 | −0.1405 | 0.5415 | 0.4855 | 1.2675 | 0.5235 | 0.3125 | 1.0145 |
| CLTC | 0.148 | 0.31 | −0.438 | −0.391 | −0.414 | −0.049 | −0.38 | 0.763 | 0.714 | 0.8 | 0.641 | −0.036 | 0.091 |
| CLU | −0.232 | 0.38 | −0.4 | −0.28 | −0.1 | 0.4 | 0.479 | 0.52 | 0.52 | 0.91 | 0 | 0.27 | 0 |
| CMAS | 0.4985 | 0.2015 | 0.6815 | 0.1425 | −0.6655 | −0.0805 | 0.1655 | 0.5585 | 0.5505 | 0.6965 | −0.7325 | 0.1225 | −0.1395 |
| CML65 | 0.147 | −0.12 | −0.815 | −0.188 | −0.013 | 0.07 | 0.506 | 0.146 | 0.095 | 0.009 | −0.020 | −0.029 | −0.01 |
| CNIH | 0.181 | −0.451 | 0.124 | −0.016 | 0.114 | 0.161 | 0.199 | −0.219 | −0.191 | −0.138 | 0.241 | −0.1 | −0.109 |
| CNOT10 | −0.3005 | −0.2715 | −0.2295 | −0.1155 | 0.7495 | −0.4685 | −0.4135 | −0.1025 | −0.1965 | 0.0105 | −0.1315 | −1.2805 | −0.4635 |
| CNOT2 | 0.385 | −0.158 | −0.063 | −0.352 | −0.323 | −0.371 | 0.158 | 0.315 | 0.37 | −0.092 | 0.492 | 0.425 | 0.007 |
| CNOT7 | −0.107 | −1.079 | −0.251 | −1.04 | −0.593 | 0.04 | −0.593 | 0.334 | 0.289 | 1.704 | −1.439 | 2.817 | 1.277 |
| CNP | 0.6255 | −0.5415 | −0.1465 | 0.0165 | 0.4505 | −0.0965 | 0.2815 | 0.4445 | 0.5035 | −0.3225 | 0.0015 | −0.5165 | 0.4505 |
| CNTNAP2 | 0.2 | −0.065 | 0.179 | −0.432 | −0.366 | −0.196 | −0.231 | −0.147 | −0.103 | −0.147 | −0.148 | 0.162 | −0.608 |
| COASY | −0.6215 | −0.2425 | 0.7475 | 0.0185 | −0.5725 | 0.4285 | 0.5825 | −2.4725 | −2.2035 | −1.6955 | −1.1275 | −1.4595 | 0.5345 |
| COL11A2 | 1.233 | −0.418 | 1.158 | 1.522 | 0.381 | 1.119 | 0.002 | 0.331 | 0.374 | 1.08 | 2.103 | −0.541 | −0.805 |
| COL27A1 | 0.283 | 2.311 | 0.779 | −0.502 | −0.697 | 0.137 | 0.007 | −0.641 | −0.593 | −0.811 | 0.428 | −0.26 | −0.63 |
| COL4A5 | −2.1745 | 1.9775 | −0.1285 | 0.7415 | −0.9635 | −0.8275 | 0.9105 | −2.1455 | −2.1705 | −2.7245 | −0.9335 | −1.1465 | 1.0145 |
| COL8A1 | 0.272 | 0.645 | 0.382 | −0.686 | 0.524 | −0.405 | 0.254 | 1.4 | 1.379 | 1.349 | −0.124 | 0.791 | 0.702 |
| COL8A3 | 0.49 | 0.173 | −0.094 | −0.231 | 0.406 | 0.06 | −0.835 | −0.026 | 0.039 | 0.189 | 0.274 | 0.654 | 0.103 |
| COMMD1 [symbol] | −0.9165 | −0.0695 | −0.2315 | −0.2635 | −0.1415 | 0.0585 | 0.0595 | 0.0175 | 0.0685 | 0.0825 | −0.0075 | −0.5485 | 0.2315 |
| COMMD5 | 0.9316 | 0.7415 | 0.7885 | 2.0095 | 1.5255 | 1.8965 | 0.0255 | −0.9355 | −0.8835 | 1.4225 | −0.3045 | −0.6935 | −0.0855 |
| COPS7B | 0.138 | −0.532 | 0.115 | −0.636 | −0.025 | −0.013 | −0.302 | 0.145 | −0.054 | −0.735 | −0.066 | 0.581 | 0.179 |
| CORO6 | 0.7505 | 0.3105 | −0.0585 | −0.1395 | −0.4445 | −0.0745 | 1.3265 | 0.6105 | 0.7445 | 0.1875 | 0.0135 | 0.6145 | 0.4105 |
| CORT | −0.223 | −0.503 | −0.259 | −0.285 | −0.412 | 0.055 | 0.075 | 0.246 | 0.28 | 0.434 | −0.008 | 0.072 | −0.244 |
| COX17 | −0.064 | −0.308 | −0.075 | −0.407 | −0.54 | −0.179 | 0.006 | 0.185 | 0.22 | 0.508 | 0.1 | −0.228 | −0.23 |
| COX5A | 0.427 | 0.467 | 0.173 | −0.527 | −0.332 | −0.196 | 0.792 | 0.619 | 0.571 | 0.303 | −0.222 | 0.908 | 0.355 |
| COX6A | 0.1995 | 0.0315 | −0.0845 | 1.2315 | 3.4255 | 1.4305 | 2.6015 | 5.1305 | 5.1455 | 2.6785 | 1.6245 | 4.0255 | 4.4215 |
| COX8A1 | −0.4015 | 0.2575 | 0.4165 | 0.2115 | 0.3705 | 0.0105 | 0.1735 | 0.3525 | 0.3855 | 0.0045 | −0.2355 | 0.3905 | 0.1755 |
| COX8C | −1.2715 | 0.2805 | −0.3295 | −1.0855 | −0.0555 | 1.9975 | −0.5615 | −1.5175 | −1.2615 | −1.0305 | −1.4985 | −0.0605 | −0.2735 |
| COX7A2 | −0.616 | −0.539 | −0.028 | −0.762 | −0.743 | −0.587 | 1.096 | 0.393 | 0.099 | −0.684 | −1.218 | 0.066 | −0.439 |
| CP | 3.1485 | 0.5495 | −0.5655 | −0.2605 | 2.8545 | 1.4535 | 3.3675 | 3.3405 | 3.2245 | 2.1185 | 2.1595 | 0.5975 | 3.1425 |
| CPAMD0 | 0.235 | 0.113 | 0.646 | −0.097 | −0.159 | 0.309 | 0.689 | 0.338 | 0.338 | 0.498 | −0.412 | 0.915 | 0.554 |
| CPB1 | 0.1495 | 1.1655 | 1.9645 | 0.5845 | 0.7145 | 0.4465 | 0.8205 | 1.1875 | 1.1455 | −0.1605 | 0.1495 | 1.9195 | 0.9425 |
| CPEB3 | −0.106 | −0.284 | 0.131 | −0.384 | −0.157 | 0.121 | −0.649 | −0.073 | −0.097 | −0.02 | −0.02 | −0.487 | −0.186 |
| CPEB4 | −0.905 | −0.601 | −0.108 | −0.477 | −0.122 | −0.342 | −0.398 | −1.147 | −0.553 | −1.051 | −0.152 | 0.466 | 0.348 |
| CPSF3 | 0.1155 | −0.4525 | 1.0775 | 0.3715 | 0.8005 | −0.3615 | 1.1535 | −1.6495 | −1.7405 | 1.0455 | −0.4605 | 0.0385 | 1.2575 |
| CRABP1 | −0.899 | −0.376 | −0.117 | 0.571 | 0.895 | 0.199 | 1.096 | 0.594 | 0.359 | 0.661 | −1.117 | 0.961 | 0.343 |
| CREB3L4 | −0.337 | −0.434 | −0.377 | −0.383 | −0.512 | −0.374 | −0.159 | −1.32 | −0.742 | −1.005 | 0.762 | 0.505 | 1.603 |
| CRIP1 | 1.403 | 0.297 | 0.063 | 0.487 | 0.881 | −0.351 | 0.395 | −0.22 | −0.255 | 0.221 | 0.555 | 0.361 | 0.231 |
| CRYBB2 | 0.817 | 0.35 | 1.141 | −0.321 | −0.427 | 0.729 | 0.15 | 0.008 | −0.008 | 0.806 | 0.933 | 0.373 | 1.02 |
| CRYL1 | −0.843 | −0.076 | −0.662 | −0.198 | 0.134 | −0.073 | −1.237 | −0.717 | −0.663 | −0.479 | −0.37 | −0.717 | −0.623 |
| CRYZ | −0.7275 | 0.3675 | −0.2505 | −0.1805 | −0.0385 | −0.2435 | −0.1985 | −0.1135 | −0.0395 | 0.1935 | −0.1405 | −0.2135 | −0.5915 |
| CSDA | −0.5435 | −0.1705 | −0.3435 | 0.2265 | 0.0925 | −0.0285 | −0.3745 | −0.0635 | −0.0265 | −0.2175 | −0.1515 | −0.3505 | 0.1875 |
| CSE1L | −0.3905 | −0.4695 | −0.1585 | 0.4595 | −0.0225 | 0.0225 | −0.7905 | −0.1395 | −0.1565 | −0.5115 | −0.7505 | −0.5325 | 0.2275 |
| CSK | 1.0295 | 0.2845 | 0.2185 | 0.9235 | −0.3445 | −0.3575 | −0.4875 | −0.5275 | −0.4685 | −0.6635 | 0.1335 | −1.7535 | −0.7315 |
| CSRP1 | 2.2385 | −2.4615 | −0.6435 | 0.9075 | 1.2735 | 1.4145 | −1.6575 | −0.5505 | −0.2835 | 1.4205 | −1.4875 | −0.3945 | 1.8125 |
| CSRP2 | 0.255 | −0.665 | −0.148 | −0.352 | −0.041 | −0.43 | −0.075 | −0.347 | −0.391 | −0.148 | −0.103 | −0.305 | 0.089 |
| CST5 | 1.3325 | 0.7705 | 0.6495 | −0.2155 | −0.1315 | −0.0235 | 0.7845 | −1.1885 | −0.8525 | −1.3395 | −0.8455 | −0.2135 | −0.9055 |
| CSTB | −0.282 | 0.241 | 0.442 | 0.123 | 0.446 | 0.75 | 0.121 | −1.328 | −1.291 | −0.143 | −1.225 | −0.198 | −0.116 |
| CTAG1B | 0.0265 | −0.5095 | −0.1485 | −0.1315 | 0.9205 | 0.0195 | 0.6235 | −0.3765 | −0.3825 | 0.0655 | 0.3075 | 0.4915 | 0.1405 |
| CTBP2 | −0.871 | 1.192 | 0.086 | 0.039 | −0.629 | 0.089 | −1.275 | −0.688 | −0.603 | −0.31 | −1.658 | 0.137 | −0.114 |
| CTNNA1 | 1.249 | 1.369 | 1.062 | −0.002 | 2.413 | 0.465 | 0.209 | 0.879 | 0.979 | −0.001 | 0.398 | −2.1 | 1.06 |
| CTNNAL1 | −0.7615 | −0.7145 | −0.6395 | 0.4815 | −0.8885 | 0.3895 | −0.8385 | −0.2925 | −0.1735 | −0.9495 | −0.0595 | −1.3685 | −0.7205 |
| CTNND2 | 0.002 | 0.691 | −1.242 | −1.412 | −1.29 | −0.62 | −0.974 | −0.205 | −0.222 | −0.131 | 0.066 | −1.056 | −0.826 |
| CTPS | −0.518 | −0.421 | −0.41 | 0.007 | 0.028 | −0.349 | −1.128 | 1.151 | 1.264 | 1.04 | −0.904 | 0.38 | −0.001 |
| CTSL2 | 0.3325 | −0.0555 | −0.6185 | −0.0325 | −0.3225 | −0.2455 | 0.0745 | 0.1195 | 0.2275 | 0.0815 | −0.1055 | −0.8445 | −0.4465 |
| CTSS | −1.47 | −0.924 | −0.29 | −1.745 | −0.471 | −0.248 | −0.35 | −1.019 | −0.973 | −0.717 | −1.408 | −0.35 | 0.58 |
| CTTN | −1.4765 | −2.0595 | −0.7125 | −1.2125 | −0.1065 | −0.0805 | −0.4755 | 2.2705 | 2.5225 | 1.4995 | −0.7665 | 2.7665 | 1.1115 |
| CX3CL1 | −0.2265 | 0.0115 | 0.7525 | −0.1795 | 0.1065 | −0.0485 | 0.0695 | −0.2135 | −0.2275 | 0.4815 | −0.1065 | 0.1565 | 0.5515 |
| CXCL9 | −0.8875 | −0.2745 | 1.4075 | 0.1025 | 1.4195 | 0.3795 | −1.5495 | 1.5345 | 1.4135 | 1.5565 | 1.4715 | 1.1535 | −0.0915 |
| CXorf39 | −0.999 | 0.586 | 0.35 | 0.115 | 0.552 | 0.324 | −0.376 | 0.383 | 0.348 | 1.448 | −0.562 | 1.329 | 0.48 |
| CXXC4 | 0.483 | 0.301 | −0.021 | −0.015 | −0.241 | −0.55 | −0.102 | 0.636 | 0.687 | 0.428 | 0.317 | 0.164 | −0.419 |
| CYB5 | −0.2015 | −0.5885 | −0.4395 | −0.3645 | 0.3165 | 0.1115 | −1.0255 | −0.4965 | −0.5285 | −0.4405 | 0.5325 | 0.8535 | 0.3145 |
| CYB561 | −0.443 | −0.426 | −0.672 | 0.175 | 0.231 | 0.37 | −0.404 | 0.271 | 0.185 | 0.037 | −0.297 | 1.382 | 0.872 |
| CYC1 | −0.211 | −0.3 | −0.189 | −0.168 | −0.002 | −0.003 | 3.376 | −0.425 | −0.384 | −0.255 | −0.044 | −0.503 | −0.244 |
| CYHR1 | 0.8565 | 4.1445 | 0.2865 | 3.7875 | 3.2995 | 0.6235 | −0.4265 | −1.4955 | −1.6755 | 1.2555 | −0.0615 | 6.0165 | 0.6555 |
| CYP2A13 | −0.12 | 0.893 | 0.36 | −0.241 | 0.028 | 0.04 | −0.064 | −0.233 | −0.159 | −0.259 | 0.004 | 0.228 | −0.114 |
| CYP4X1 | 2.4855 | 1.0145 | 1.0935 | 0.5075 | 0.4845 | 0.2265 | 2.4775 | 1.1805 | 2.2225 | 1.7615 | 2.4795 | 0.8275 | 1.4275 |
| D15Wsu7 [symbol] | 1.8525 | −1.0855 | 1.4495 | 0.8265 | 0.9155 | 0.7235 | 1.8105 | 1.9845 | 1.4765 | 2.5065 | 1.3295 | 0.1075 | 0.1175 |
| DACH1 | 2.972 | 1.102 | 0.747 | 0.464 | 0.759 | 0.545 | 2.633 | 2.041 | 2.628 | 2.007 | 3.074 | 0.773 | 1.725 |
| DACH1 | −0.235 | −0.367 | −0.039 | −0.052 | 0.424 | −0.258 | 0.623 | −0.268 | −0.377 | 0.692 | 0.589 | −0.681 | −1.575 |
| DACH1 | −0.132 | −0.328 | −0.891 | −0.157 | −0.69 | −0.396 | −0.129 | 0.645 | 0.787 | 0.72 | −0.193 | −0.045 | −0.359 |
| DAP3 | 0.4695 | 0.7835 | 0.5515 | −0.0475 | 0.3735 | 0.1235 | 0.9085 | 0.3075 | 0.4355 | 0.6455 | −0.3035 | −0.7705 | 0.9875 |
| DAPP1 | −0.789 | 1.183 | 0.301 | −0.09 | −0.406 | 0.034 | −1.326 | −0.502 | −0.595 | 0.365 | −0.963 | −0.33 | −1.224 |
| DAZAP2 | 0.245 | −0.418 | 0.059 | 0.167 | −0.096 | −0.025 | −0.36 | 0.674 | 0.668 | −0.178 | 0.346 | −0.194 | −0.852 |
| DBI | −0.2605 | −0.8155 | −0.1225 | −0.8875 | −1.0245 | −0.4355 | −0.4455 | −0.2335 | −0.1825 | 0.0135 | 0.3425 | −0.5985 | −0.4615 |
| D9N1 | 0.739 | 0.377 | −0.916 | −0.181 | −0.19 | −0.66 | 0.036 | −0.281 | −0.226 | 0.056 | 0.156 | −0.205 | −0.552 |
| DC13 | 2.6005 | 0.4705 | −0.0965 | 0.1695 | 0.1285 | −0.3745 | −0.3395 | −0.2485 | −0.0175 | −0.1715 | 1.9915 | −0.5695 | 1.7905 |
| DCPS | −0.7355 | 0.1075 | 0.0885 | 0.4525 | 0.3315 | 0.9265 | 0.4265 | 1.2005 | 1.3545 | −0.0315 | 1.5395 | 0.8985 | 0.4705 |
| DDHD2 | −0.8545 | 0.2085 | −0.7645 | −0.2625 | −0.4165 | −0.1805 | −0.2655 | −0.0665 | −0.1195 | −0.5955 | −0.1155 | −0.4195 | −0.0515 |
| D0X17 | 0.8515 | 0.3165 | 0.0555 | −0.3235 | −0.1925 | 0.2285 | −0.1245 | 0.4385 | 0.4025 | 0.0515 | 0.9995 | −0.7065 | −0.2845 |
| DDX39 | −0.358 | 0.119 | −0.07 | −1.03 | −0.859 | 0.508 | −0.059 | 1.264 | 1.271 | 0.29 | 1.06 | −0.301 | 0.111 |
| DDX48 | −0.8005 | −0.1365 | −0.0265 | −0.6375 | −0.1405 | −0.0605 | 1.0995 | 0.3865 | 0.2735 | 0.3205 | −0.0675 | 1.0165 | 0.1855 |
| DDX58 | 0.1005 | −0.0695 | 0.2945 | −0.0955 | −0.3865 | −0.3765 | −1.0685 | −0.0735 | −0.0335 | −0.1885 | −0.6165 | −0.7715 | −0.2375 |
| DEF6 | −1.120 | −0.925 | −0.644 | −0.273 | −0.674 | −0.296 | −0.728 | −0.603 | −0.695 | −0.388 | −0.84 | −1.553 | −1.27 |
| DEK | 1.178 | 0.622 | 0.352 | 0.195 | −0.262 | −0.657 | −0.642 | 0.125 | 0.044 | 1.05 | 0.148 | 1.227 | 0.468 |
| DHCR7 | −1.175 | 1.022 | −0.949 | −0.243 | −0.457 | −0.25 | −0.545 | −0.143 | −0.005 | 0.079 | −0.623 | −0.449 | −0.362 |
| DHRS7 | −0.3905 | 0.0775 | −1.2685 | 0.4875 | −0.0675 | 0.1115 | −0.5655 | 0.2665 | 0.1575 | 0.1605 | 0.2325 | −0.3835 | −0.6695 |
| DHX57 | 1.5215 | 1.4625 | 0.8375 | 0.5935 | 1.3445 | 0.8185 | 1.5215 | −0.4855 | −0.5195 | 1.5945 | −1.6835 | 2.4055 | 0.6155 |
| DIRC1 | 0.627 | −0.354 | −0.349 | −0.139 | 0.118 | −0.289 | 0.513 | −0.439 | −0.428 | −0.521 | 0.238 | −0.557 | 0.196 |
| dJ222E13 | 0.158 | −0.505 | 0.094 | −0.508 | −0.404 | −0.128 | −0.581 | −0.17 | −0.076 | −0.183 | −0.691 | −0.164 | −0.192 |
| DKFZP434 | 1.09 | −0.006 | −1.369 | −0.406 | −0.432 | −0.122 | −0.613 | −0.556 | −0.392 | −0.573 | −0.152 | −0.688 | 0.838 |
| DKFZp434 | −0.6695 | −0.5135 | −0.0425 | −0.4035 | −0.5835 | 0.0615 | −0.4145 | −1.5375 | −1.2585 | −1.3125 | 0.1555 | −0.4845 | −0.8605 |
| DKFZP434 | 0.012 | −0.261 | −0.483 | 0.271 | −0.074 | −0.465 | 1.283 | −0.267 | −0.216 | −0.349 | 0.304 | −0.481 | 0.418 |
| DKFZp434 | 0.0275 | 0.2335 | 0.2925 | 0.0885 | 0.2365 | 0.2545 | 0.6975 | 0.4665 | 0.5385 | 0.5665 | 0.3845 | −0.0275 | 0.2675 |
| DKFZP564 | −0.1055 | 0.1115 | −0.1405 | −0.0785 | 0.3315 | −0.1165 | −0.0395 | 0.1385 | 0.2045 | 0.3655 | −0.0895 | −0.0655 | 0.2955 |
| DKFZp564 | −0.07 | −0.005 | −0.738 | −0.557 | −0.170 | 0.282 | −0.072 | 0.682 | 0.756 | 0.705 | −0.001 | 0.348 | 0.185 |
| DKFZP564 | 0.6545 | 0.2125 | −0.1975 | 0.2495 | 0.3715 | 0.0735 | 0.9525 | 0.4495 | 0.3895 | 0.2415 | −0.2495 | −0.1935 | 0.0535 |
| DKFZP564 | 0.836 | 0.386 | 0.324 | 0.578 | 0.169 | −0.182 | −0.675 | 0.027 | 0.175 | 0.236 | −0.239 | −0.567 | −1.057 |
| DKFZP564 | −0.262 | 0.935 | 0.993 | 0.805 | 2.364 | 0.22 | −1.374 | −1.047 | −1.045 | 0.208 | −1.319 | −1.733 | 0.066 |
| DKFZp586 | −0.5535 | −1.1285 | 1.1225 | 0.7545 | 0.4025 | 0.6555 | −0.7705 | −1.2295 | −1.0685 | −1.0645 | −0.0235 | −0.7845 | −0.5745 |
| DKFZp761 | 0.124 | −0.147 | −0.136 | 0.176 | 0.078 | −0.25 | −0.169 | −0.6 | −0.387 | −0.357 | −0.021 | −0.425 | −0.17 |
| DLK1 | 0.057 | −0.175 | 0.122 | −0.041 | −1.32 | 0.171 | 0.756 | 1.38 | 1.232 | 0.437 | 2.06 | −0.294 | 1.23 |
| DLX5 | 1.3885 | −1.3245 | −0.4575 | −0.0825 | −0.1035 | −0.7075 | −0.6065 | −0.7925 | −0.7925 | 0.5255 | −0.4815 | 2.3975 | 0.2695 |
| DNAJA4 | 1.4095 | 0.7335 | −0.4245 | 1.9345 | 1.4015 | 1.2335 | 3.3285 | 3.2465 | 3.0535 | 2.6335 | 2.1475 | 0.6025 | 2.5555 |
| DNAJC1 | 0.778 | 0.297 | −0.24 | 0.029 | 0.259 | −0.731 | −0.11 | −1.233 | −1.125 | 0.349 | −1.003 | −0.073 | 0.194 |
| DNAJC12 | 1.394 | −2.505 | 1.054 | 0.95 | 0.759 | 0.86 | 1.618 | 2.209 | 1.966 | 1.35 | 0.134 | 1.069 | 1.97 |
| DNAJD1 | −0.023 | 0.894 | 0.084 | 0.225 | −0.099 | 0.122 | −0.803 | 0.041 | 0.023 | −0.806 | −0.077 | −0.05 | 0.245 |
| DNALI1 | −0.623 | 0.547 | 0.02 | −0.111 | −0.17 | 0.165 | 0.212 | 1.466 | 1.501 | 1.011 | −0.038 | 0.072 | −0.019 |
| DNMT1 | −0.2565 | −0.0485 | 0.8545 | −0.9195 | −1.0775 | −0.2455 | −0.0675 | −0.5015 | −0.5175 | −0.5665 | −1.3215 | −0.9925 | 0.4615 |
| DOX1 | 0.2525 | −0.1555 | 0.8615 | −0.1925 | −0.5135 | 0.0225 | −0.3295 | 0.0245 | 0.1165 | −0.0355 | 0.0725 | 0.1615 | 0.4075 |
| DSCR1 | −0.0185 | −0.0755 | 0.4915 | −0.0845 | 1.8805 | 1.1655 | −1.2155 | −0.9515 | −0.8505 | 0.0335 | −0.7765 | 0.9155 | 1.3025 |
| DSCR1L2 | 0.2595 | −0.3035 | 0.1605 | −0.2875 | −0.1545 | −0.0585 | −0.4485 | −0.1015 | −0.0325 | 0.0025 | 0.0655 | −0.3275 | 0.0095 |
| DSP | 0.0775 | 0.2295 | 0.3935 | −0.1035 | 0.2855 | −0.1145 | 0.9865 | 0.4785 | 0.3685 | 0.5275 | 1.1895 | −0.3545 | −0.4645 |
| DTYMK | −0.8855 | 0.8915 | 0.1425 | 0.6045 | 2.5745 | −0.1015 | 0.2095 | 0.4755 | 0.3195 | −0.0945 | −0.5525 | 0.3415 | 0.0155 |
| DUSP23 | 1.2355 | 0.5515 | 1.4745 | −0.5595 | −0.6975 | 0.9395 | −0.3785 | −0.5245 | −0.4215 | −0.8195 | −0.6055 | −1.7475 | 1.0925 |
| DUSP4 | 0.4105 | −0.0315 | 0.0055 | 0.0595 | 0.6345 | −0.2435 | −0.1225 | −0.6825 | −0.8005 | −0.7895 | 0.2795 | −0.1065 | 0.3095 |
| DUSP5 | −0.082 | 0.036 | 0.238 | −0.313 | −0.109 | −0.154 | −0.286 | −0.052 | 0.076 | −0.236 | −0.049 | 0.252 | 0 |
| DVL1 | −0.257 | −0.159 | −0.348 | −0.008 | −0.217 | 0.173 | −0.073 | −0.238 | −0.101 | −0.402 | −0.259 | −0.288 | −0.699 |
| DXYS155 [symbol] | −0.183 | −0.096 | −0.335 | −0.201 | 0.062 | 0.565 | 0.204 | 0.487 | 0.429 | 0.995 | −0.878 | 0.617 | 0.018 |
| DYRK4 | 0.0026 | 0.4295 | 1.4355 | −0.2735 | −0.6275 | −0.3135 | −0.6535 | 0.6285 | 0.5035 | 0.9635 | −0.4435 | −0.0925 | 0.1075 |
| o(y)2 | 0.565 | −0.249 | −0.242 | −0.108 | −0.104 | −0.119 | −0.288 | 0.595 | 0.468 | −0.305 | 0.189 | 0.624 | −0.183 |
| EAF2 | 0.2215 | 1.2615 | 0.3635 | 0.5885 | −0.7485 | 0.3045 | 0.1005 | 0.2545 | 0.3105 | −0.0745 | −0.2545 | −1.0615 | 0.6035 |
| EAP30 | −0.2775 | −0.2025 | −0.1345 | −0.4405 | −0.4275 | −0.3625 | −0.8715 | 0.1185 | 0.0135 | −0.3855 | −2.2095 | 1.1315 | 1.1445 |
| ECHDC1 | 0.3975 | 1.1555 | −0.6035 | −0.1925 | 0.1195 | 0.2605 | 0.9785 | 1.8875 | 2.0505 | 0.5305 | 2.6705 | −1.1035 | 0.5595 |
| ECHDC3 | 0.5125 | 0.1725 | 0.1545 | 0.5475 | 0.7555 | −0.0595 | 0.4115 | 0.2015 | 0.2215 | 0.2805 | 0.2905 | 0.0055 | 0.5505 |
| EEF1A2 | −0.965 | 0.469 | −0.497 | −0.1 | 0.712 | −0.347 | −0.056 | −0.744 | −0.54 | 0.772 | −1.015 | −0.097 | 0.506 |
| EFHC1 | 0.2745 | 0.6015 | −0.1805 | 0.2495 | −1.0835 | −0.4195 | −1.2085 | 0.0585 | 0.1395 | −0.5385 | −2.3105 | −1.3685 | 0.4005 |
| EFNA4 | −0.3785 | −0.4105 | −0.0075 | 0.0415 | −0.1545 | 0.4665 | −0.1115 | −0.7295 | −0.7025 | −0.8175 | −0.0345 | −0.6935 | −0.1615 |
| EFS | 0.4855 | 0.5425 | 0.4865 | −0.2055 | −0.0355 | −0.0285 | 0.0605 | 0.3725 | 0.2115 | 0.0655 | 0.2215 | 0.1675 | 0.8575 |
| EIF2C2 | 1.0145 | −1.5785 | −0.7175 | −0.3895 | −0.4625 | −0.6555 | −1.0565 | −0.5725 | −0.5375 | 0.5655 | 1.2105 | −0.8875 | −0.1255 |
| EIF2C4 | −0.125 | −0.042 | −0.554 | 0.306 | 0.023 | −0.117 | −0.38 | 0.014 | 0.077 | −0.433 | −0.083 | −0.076 | −0.446 |
| EIF4EBP1 | 0.4775 | 0.2065 | −0.0605 | 1.0455 | 0.3375 | 0.4965 | −0.4015 | 0.1535 | 0.1605 | 0.0895 | 0.3005 | −0.4935 | −0.2955 |
| EIF4G1 | −0.015 | 0.308 | 0.496 | 0.028 | −0.213 | 0.055 | 0.008 | 0.608 | 0.499 | 0.443 | 0.224 | −1.018 | 0.24 |
| EIF4G3 | 0.638 | −0.741 | 0.018 | −0.451 | −0.09 | 0.576 | −0.361 | −0.215 | −0.197 | 0.171 | −1.467 | 0.51 | 0.585 |
| ELAC1 | 0.794 | −0.923 | −0.653 | −0.342 | −0.521 | 0.018 | 0.504 | 0.695 | 0.593 | 0.719 | −0.243 | 0.372 | 0.658 |
| ELL2 | 0.7505 | 1.8355 | 1.0915 | 1.0435 | 1.2385 | 0.9915 | −1.0655 | 1.3945 | 1.2785 | −0.1435 | −0.4265 | 2.4145 | 0.7685 |
| ELL3 | 0.4885 | −0.4775 | −0.5175 | 0.5405 | 0.1835 | −0.0635 | −0.6855 | −0.8275 | −0.2885 | −0.3035 | −0.1445 | 1.8375 | −0.5925 |
| ELOVL5 | 0.0225 | −0.3505 | 0.9285 | 1.1775 | 0.4995 | 0.5235 | −0.2495 | −0.0445 | −0.0805 | 0.5635 | −0.1585 | −0.9935 | −0.7165 |
| EN1 | −0.2805 | 0.5075 | −0.2265 | 1.1565 | 1.2335 | −0.4305 | −0.2465 | 0.2775 | 0.1925 | 1.5625 | −1.0775 | 0.3175 | −0.3615 |
| ENAH | 0.2935 | 1.2655 | 0.3515 | 0.8705 | 0.4075 | 0.3625 | −0.5485 | −0.1705 | 0.0245 | 0.5745 | −0.2905 | −0.2195 | 0.3215 |
| EPHX2 | 0.411 | 0.404 | −0.085 | −0.76 | −0.206 | 0.106 | 1.406 | 1.333 | 1.332 | 2.125 | 0.94 | 0.932 | 1.089 |
| EPLIN | 0.6005 | 0.6565 | 0.5085 | 0.8495 | 1.1025 | 0.4045 | 1.5876 | 0.3145 | 0.3865 | 0.9215 | 0.3235 | −1.3705 | 1.8985 |
| EPN3 | 1.0685 | 1.0535 | −0.1815 | −0.2995 | 0.3495 | −0.2575 | 0.0005 | 0.2625 | 0.3155 | 0.2675 | −0.7135 | 0.0325 | 0.4765 |
| EPOR | 0.595 | 0.21 | −0.3 | 0.281 | −0.065 | −0.252 | −0.174 | 0.908 | 0.81 | 0.278 | −0.049 | −0.416 | −0.547 |
| ERBB2 | 0.8785 | −0.4945 | 1.8535 | 2.2755 | 1.0715 | 0.5185 | 2.0115 | 0.9505 | 0.8045 | 1.6015 | 3.0375 | 2.9715 | 3.1335 |
| ERP70 | 0.976 | 0.466 | 0.303 | 0.245 | −0.643 | −0.271 | 0.361 | −0.234 | −0.273 | −0.1 | −0.133 | −0.378 | −0.708 |
| ESR1 | −0.3615 | −1.1435 | −0.5165 | 0.0915 | −0.1575 | 0.1785 | −0.4585 | −0.5545 | −0.4715 | −0.2905 | −0.6905 | −0.4095 | −0.8295 |
| ETFA | −0.2405 | 0.1685 | −1.2225 | −0.1565 | −0.0295 | −0.6315 | −0.9385 | 0.9025 | 0.8515 | 0.0255 | 0.2716 | 0.5225 | −0.7875 |
| ETV8 | −0.6475 | −0.9935 | −0.9005 | −0.4435 | 0.1865 | −0.0235 | −1.3355 | −0.1935 | −0.1535 | −1.0635 | −0.2735 | −0.3665 | −0.8395 |
| EVER1 | 0.3235 | 0.1665 | −0.2195 | −0.4275 | −0.2025 | −0.1425 | 0.2665 | 0.1235 | 0.0945 | 0.0715 | −0.0505 | −0.1535 | −0.2485 |
| EXO1 | 0.8285 | −0.2465 | 0.2275 | 0.2045 | 0.0945 | 0.0545 | −1.3895 | −0.0235 | −0.0395 | −0.0685 | −0.3905 | 0.1125 | −0.6615 |
| EXOSC9 | −1.397 | −1.383 | −0.466 | −0.834 | −0.702 | −0.113 | −0.837 | 0.032 | −0.137 | −0.692 | 0.438 | −0.115 | −0.423 |
| EXT2 | 0.59 | 0.6 | 0.506 | 0.365 | 0.566 | −0.268 | 0.87 | 0.487 | 0.374 | 1.074 | 0.3 | 0.665 | 0.279 |
| EZH2 | −0.1275 | −0.3265 | 0.3455 | −0.1215 | 2.6415 | −0.2025 | −0.1995 | −0.1575 | −0.0245 | −0.5805 | 0.0075 | −0.0475 | −0.0035 |
| FI1R | 2.372 | 0.282 | −0.580 | −0.639 | −1.129 | −0.251 | −0.209 | −0.326 | −0.27 | −0.058 | 0.577 | −1.922 | −1.075 |
| F9 | −0.619 | −0.709 | −0.126 | −0.178 | −0.595 | 0.342 | −0.447 | −2.477 | −1.759 | −1.842 | −0.658 | −1.517 | −0.07 |
| FA2H | 0.164 | −0.499 | −0.331 | −0.117 | −0.333 | −0.191 | 0.81 | −0.132 | −0.05 | −0.243 | 0.031 | −1.073 | 2.143 |
| FABP7 | 0.432 | −0.594 | 0.29 | −0.353 | 0.178 | −0.012 | 0.667 | −0.024 | −0.105 | 0.267 | 0.256 | 0.541 | 1.361 |
| FADD | −0.024 | −0.011 | −0.078 | −0.289 | −0.057 | −0.101 | −0.197 | −0.62 | −0.594 | −0.081 | −0.456 | −0.211 | 0.018 |
| FAHD1 | −0.223 | 0.245 | 0.677 | −0.09 | −0.098 | −0.036 | 0.308 | 0.826 | 8.865 | 0.492 | 0.088 | −0.052 | −0.827 |
| FAHD2A | −0.188 | 0.11 | −0.725 | −0.174 | −0.178 | −0.306 | −0.373 | 0.4 | 0.446 | 0.237 | 0.606 | 0.195 | 0.322 |
| FAM38A | −0.6715 | 1.1235 | 1.9595 | 3.7385 | −0.5575 | −0.4695 | 2.8205 | 3.7445 | 3.6245 | 0.4115 | −0.0005 | −1.3405 | 0.7155 |
| FAM3A | 0.7445 | 0.3095 | −0.5305 | 0.2585 | −0.4285 | −0.2405 | −0.0016 | 1.1015 | 1.0015 | −0.1305 | 0.3365 | −0.1475 | −0.3475 |
| FAM3B | 0.982 | 0.157 | −0.459 | −0.076 | −0.886 | 0.472 | 0.005 | 0.891 | 0.828 | −0.073 | −0.801 | 1.141 | 0.44 |
| FAM3C | −0.435 | −0.116 | −0.29 | −0.407 | −0.471 | 0.059 | 0.307 | 0.685 | 0.79 | −0.106 | 0.131 | −0.036 | −0.099 |
| FAM46C | −1.196 | 1.381 | −1.412 | −1.047 | −0.644 | −0.073 | −1.158 | −0.673 | −0.603 | −1.137 | 0.207 | 0.169 | −0.051 |
| FAM49B | −1.432 | 0.627 | −1.492 | −0.693 | −0.743 | −0.353 | −0.958 | −0.853 | −0.723 | −1.273 | 0.307 | 0.143 | −1.034 |
| FAM54A | −0.536 | −0.927 | −0.363 | −0.99 | −0.628 | −0.474 | 0.419 | 0.372 | 0.387 | −0.386 | −0.56 | −0.036 | −0.111 |
| FANCA | −0.2645 | −0.0645 | 0.0845 | −0.3475 | 0.2075 | 0.0655 | −0.2865 | 8.1085 | 0.3345 | −0.0015 | −0.1205 | −1.1795 | −0.6415 |
| FANCE | −0.0215 | 1.4325 | 1.3575 | −0.6395 | −0.4605 | −0.1045 | 1.8415 | 1.3835 | 1.4155 | 0.8095 | 0.0465 | 1.2985 | 1.0915 |
| FBL | 0.163 | −0.238 | −0.17 | −0.118 | 0.26 | −0.136 | 0.195 | −0.096 | −0.065 | −0.056 | 0.155 | 0.689 | 0.392 |
| FBP1 | −0.272 | −0.371 | 0.142 | −0.195 | −0.182 | −0.172 | 0.012 | 0.145 | 0.26 | −0.307 | −0.155 | −0.353 | −0.119 |
| FBS1 | −0.2505 | 0.9045 | −0.1895 | −0.0865 | 0.9155 | −0.5175 | 0.3185 | −0.1925 | −0.1325 | 0.4445 | −0.7395 | −0.6975 | −0.1315 |
| FBXL20 | 1.086 | 0.305 | −0.27 | −0.221 | −0.37 | 0.011 | 0.854 | 1.03 | 1.059 | 0.227 | 1.57 | 0.301 | 0.608 |
| FBXO25 | 0.581 | 0.450 | 0.478 | −0.106 | −0.003 | −0.004 | −0.176 | −0.025 | −0.149 | 0.147 | 0.028 | −0.831 | 0.492 |
| FDXR | 0.524 | 0.312 | 0.52 | −0.171 | −0.377 | 0.173 | −0.293 | 1.465 | 1.503 | −0.007 | −0.19 | −1.802 | −0.39 |
| FEM1B | −0.362 | 0.218 | 1.707 | −0.185 | −1.89 | −0.762 | −2.235 | 0.073 | 0.239 | 0.128 | 0.651 | 2.038 | −1.228 |
| FGF12 | −0.1965 | −0.4495 | −0.4735 | 0.1245 | −0.2815 | −0.0855 | −0.2895 | −0.9385 | −0.8955 | −0.2245 | 0.5175 | −0.1105 | −0.9615 |
| FGF13 | 0.34 | −0.529 | −0.299 | 0.436 | −0.127 | 0.088 | −0.704 | −0.084 | −0.106 | 0.646 | −0.006 | 0.077 | −0.02 |
| FHOD1 | 1.2055 | −0.0255 | −0.1895 | 1.8355 | 2.2805 | 0.6825 | 0.3055 | −0.4265 | −0.7365 | 1.7525 | −0.2605 | 0.0145 | −0.2565 |
| FKBP11 | 0.3155 | 1.3135 | 0.2775 | 0.5185 | 0.2045 | 0.4865 | −0.5215 | 1.0505 | 0.8955 | 0.7775 | 0.4835 | 0.8025 | 1.0325 |
| FLJ10094 | 0.83 | 0.136 | 0.509 | 0.883 | 0.376 | 0.602 | −0.3 | 0.184 | 0.164 | −0.429 | −0.284 | −0.639 | −0.648 |
| FLJ10115 | −0.3345 | 0.0545 | −0.1235 | −0.3125 | −0.0675 | 0.6385 | 0.3075 | 0.9305 | 0.8275 | 0.2585 | −0.0705 | 0.7865 | −0.0295 |
| FLJ10159 | −0.267 | 0.216 | −0.066 | −0.249 | −0.309 | −0.2 | 0.25 | −0.015 | −0.003 | −0.404 | 0.102 | 0.187 | 0.496 |
| FLJ10204 | −0.106 | −0.741 | 0.352 | −0.201 | −0.557 | 0.033 | 0.357 | 0 | 0.041 | 0.171 | 0.041 | −0.193 | −0.126 |
| FLJ10275 | 1.7935 | 0.8945 | 1.7185 | −0.5835 | −0.4755 | −0.2415 | 2.1635 | 3.0165 | 2.9735 | 2.5585 | 1.7645 | −0.1005 | 0.7085 |
| FLJ10292 | 0.4695 | 0.1365 | 0.6915 | −0.0345 | 0.0685 | 0.3575 | 0.1645 | 0.1105 | −0.0305 | 0.0215 | 0.1395 | −0.4265 | 0.2515 |
| FLJ10324 | −0.6255 | −0.5135 | −0.1615 | −0.3895 | 0.1725 | 0.2425 | −0.1185 | −0.7695 | −0.6335 | −0.1135 | 0.0765 | −0.4385 | −0.7075 |
| FLJ10700 | −0.309 | 0.412 | 0.012 | 0.248 | 0.749 | −0.116 | −0.385 | −0.053 | −0.048 | −0.008 | −0.435 | −0.175 | −0.253 |
| FLJ10706 | −1.289 | 0.018 | −1.368 | −0.679 | −1.088 | 0.247 | −0.623 | 0.975 | 0.708 | −1.035 | −1.13 | −0.689 | −0.845 |
| FLJ10671 | 0.799 | −1.238 | 0.306 | −0.192 | 0.363 | 0.883 | 0.965 | 0.959 | 0.69 | 0.001 | 0.138 | −0.387 | 0.598 |
| FLJ10901 | 0.8865 | 0.9965 | 1.7075 | 0.3225 | 0.2465 | 1.1615 | −0.2455 | −0.1095 | −0.0925 | 0.0755 | −0.6675 | 1.4845 | 1.0045 |
| FLJ10918 | 1.207 | −0.036 | −0.55 | −1.02 | −0.993 | −0.338 | 0.659 | −0.509 | −0.328 | 0.323 | 0.942 | −0.911 | −1.079 |
| FLJ10980 | 0.3245 | 0.5165 | 1.0105 | 0.1755 | −0.0135 | 0.4545 | 1.4055 | 1.0245 | 1.0235 | 0.6145 | 0.0855 | −0.4955 | 0.7485 |
| FLJ11017 | 0.3555 | 0.1275 | −0.0445 | 0.4145 | 0.3795 | 0.5155 | −0.2535 | −0.2495 | −0.0845 | 0.3915 | 0.1065 | 0.3345 | 0.3855 |
| FLJ11088 | 0.0045 | 1.0375 | 0.0665 | 0.1385 | 0.6515 | 0.7265 | −0.5145 | 0.3995 | 0.2935 | −0.2175 | 0.3915 | −0.0295 | −0.8685 |
| FLJ11151 | 0.3515 | −0.4595 | 0.1825 | 0.0745 | 0.5015 | 0.9065 | 0.2185 | 0.6745 | 0.5935 | 0.6185 | 0.5775 | 0.2605 | 1.2255 |
| FLJ11267 | −0.0995 | 0.0515 | 0.2715 | 0.1585 | 0.0165 | 0.2715 | 0.4305 | 0.3375 | 0.3025 | 0.3415 | −0.1135 | −0.2855 | 0.6775 |
| FLJ11280 | −0.0095 | 0.0405 | −0.8065 | −0.2475 | −0.3135 | −0.1665 | 0.5465 | −0.3475 | −0.2595 | −0.3335 | 0.7035 | −0.1295 | −0.3975 |
| FLJ11508 | 0.6005 | 0.5755 | −0.0865 | 0.0795 | −0.1735 | −0.1805 | 0.0175 | −0.6875 | −0.8845 | 0.2925 | −0.9575 | −0.7605 | 0.1375 |
| FLJ12270 | 1.8645 | −0.7705 | 1.6545 | 0.7155 | 1.6245 | 2.3315 | 2.3455 | 4.4165 | 4.3925 | 1.9975 | 3.7685 | 1.1465 | −0.2525 |
| FLJ12844 | 0.8725 | −0.2885 | −0.3265 | −0.0315 | −0.2945 | −0.0945 | 0.0795 | 0.5795 | 0.2465 | −0.3165 | −0.3195 | 0.2975 | −0.1165 |
| FLJ12650 | 0.2395 | −0.3025 | −0.0805 | −0.1395 | −0.1705 | −0.0325 | −0.5255 | −0.4745 | −0.7125 | −0.3805 | −0.1195 | −0.0535 | −0.5805 |
| FLJ12684 | −0.7265 | −0.3785 | −0.6885 | −0.0885 | −0.0565 | −0.0835 | −0.3625 | −0.6375 | −0.6295 | −0.0105 | −0.6575 | 0.3185 | −0.6775 |
| FLJ12735 | 0.977 | 0.367 | 0.387 | 0.655 | 1.061 | 0.203 | −0.373 | 0.114 | −0.014 | 0.035 | 0.826 | 0.835 | 0.106 |
| FLJ12750 | 0.861 | 1.427 | 1.965 | 0.874 | 0.646 | 0.794 | 1.979 | 1.302 | 1.442 | 0.176 | 2.432 | 1.473 | 1.833 |
| FLJ12895 | 0.7155 | 0.8175 | −0.1745 | −0.5765 | −0.2305 | −0.1475 | 0.1055 | 0.6855 | 0.6095 | −0.0575 | 0.0295 | −0.7595 | 0.0155 |
| FLJ13710 | 0.2745 | −0.5895 | 0.2175 | −0.3985 | −0.5995 | −0.1425 | −0.5125 | −0.2335 | −0.2785 | −0.0135 | 0.2445 | −1.3985 | −0.6745 |
| FLJ13855 | −0.347 | −0.491 | −0.193 | −0.068 | 0.321 | 0.279 | 0.271 | −0.837 | −0.623 | −0.25 | 0.15 | −0.317 | −0.946 |
| FLJ13912 | −0.105 | 0.637 | −0.541 | −0.617 | −0.65 | −0.205 | −0.689 | 0.438 | 0.519 | 0.07 | 0.117 | −1.211 | 0.045 |
| FLJ14124 | −0.39 | 0.591 | −0.487 | 0.191 | −0.242 | 0.017 | −0.793 | −0.05 | −0.037 | −0.116 | 0.354 | −0.709 | −0.505 |
| FLJ14627 | −0.0015 | −0.0415 | 0.1585 | −0.1365 | 0.0425 | 0.0795 | −0.0005 | −0.0695 | −0.2375 | −0.0605 | 0.0405 | −0.0485 | −0.0525 |
| FLJ14666 | −0.55 | −0.823 | −0.017 | 0.089 | −0.611 | 0.202 | −0.084 | 2.882 | 2.684 | 1.031 | 2.256 | 0.629 | 0.391 |
| FLJ14904 | 0.627 | −0.052 | 0.094 | 0.061 | −0.244 | 0.237 | 0.62 | 1.134 | 1.165 | 2.177 | 1.589 | 1.825 | 1.233 |
| FLJ20035 | 0.212 | −0.151 | −0.246 | −0.437 | 0.044 | 0.213 | 0.336 | 0.12 | 0.198 | 0.136 | −0.507 | 0.517 | 0.058 |
| FLJ20152 | −0.354 | 0.489 | −0.087 | −0.091 | −0.128 | 0.278 | 0.199 | −0.043 | 0.022 | −0.377 | 0.041 | −0.088 | 0.075 |
| FLJ20171 | 0.472 | 1.535 | 0.722 | 0.745 | 0.813 | 1.051 | 1.321 | 1.187 | 1.114 | 0.638 | 0.111 | 2.104 | 1.214 |
| FLJ20244 | −0.847 | 1.503 | −0.223 | 0.296 | 0.034 | −0.544 | 0.973 | 1.404 | 1.45 | 2.426 | 1.649 | 0.577 | −0.121 |
| FLJ20273 | −0.204 | 0.055 | 0.485 | 0.165 | −0.079 | 0.033 | −0.253 | 0.062 | 0.141 | 0.192 | 0.153 | 0.24 | −0.11 |
| FLJ20315 | 0.469 | −0.516 | 0.515 | 0.037 | 0.201 | 0.296 | −0.677 | 0.287 | 0.271 | 0.052 | −0.053 | −0.614 | −0.386 |
| FLJ20323 | 0.1455 | 0.0655 | 0.1605 | −0.4315 | 0.1165 | 0.2275 | −0.5615 | 0.0085 | 0.2085 | 0.1955 | 0.1865 | −0.1765 | 0.5015 |
| FLJ20518 | 0.961 | 1.256 | 1.531 | 1.738 | −0.05 | 0.236 | −0.546 | −0.025 | −0.036 | 0.098 | −0.719 | 0.324 | 0.256 |
| FLJ20530 | 0.328 | −0.525 | −0.326 | 0.16 | −0.448 | 0.001 | 0.215 | −0.387 | −0.404 | −0.26 | 0.199 | −0.083 | −0.592 |
| FLJ20696 | 0.0965 | 0.1315 | −0.1875 | −0.5845 | 0.3565 | 0.4335 | 0.6785 | 0.8115 | 0.1505 | 0.1565 | 0.3225 | 0.5215 | 0.8605 |
| FLJ20718 | 0.081 | −0.002 | −0.423 | −0.189 | 0.157 | 0.19 | −0.952 | 0.174 | 0.11 | −0.335 | 0.464 | 0.37 | 0.256 |
| FLJ20772 | 0.3945 | −0.0275 | 0.8175 | 0.0995 | 0.6835 | 0.3425 | 0.5115 | 0.5285 | 0.5105 | −0.2585 | 0.3735 | −0.6495 | −0.5025 |
| FLJ20989 | 0.5395 | 0.5076 | 0.1195 | 0.1535 | 0.8385 | −0.4995 | 0.3915 | 0.2545 | 0.2305 | 0.4015 | −0.0835 | 1.5195 | 0.6535 |
| FLJ21019 | 1.1485 | −0.4525 | 1.6625 | 0.4515 | −0.2305 | 0.1905 | −1.7915 | 1.6795 | 1.8785 | 0.1805 | 1.0755 | −2.4115 | 0.8715 |
| FLJ21062 | −0.0085 | −0.3015 | −1.4485 | 0.9645 | −0.7545 | 0.5545 | −0.1465 | −0.0835 | −0.1895 | −0.0655 | −0.1695 | −0.4155 | 0.3715 |
| FLJ21159 | 0.8155 | 1.4185 | 0.0275 | 0.7245 | 0.7775 | −0.1475 | 1.0505 | 0.1385 | 0.1265 | 1.1425 | −0.1245 | −0.4515 | 0.0735 |
| FLJ21616 | 1.278 | 0.819 | 0.232 | 0.373 | −1.057 | 0.141 | −1.864 | −0.28 | −0.293 | 0.622 | 0.858 | −0.96 | −0.265 |
| FLJ21827 | 0.4325 | 1.0205 | 0.0885 | 0.0435 | −0.3945 | −0.5855 | −1.0375 | 0.5145 | 0.6755 | 0.3975 | −0.5235 | −0.3955 | 0.4845 |
| FLJ21963 | 0.035 | 0.991 | −0.38 | 0.258 | 0.53 | −0.074 | 0.449 | 1.335 | 1.237 | −0.115 | 0.269 | −0.767 | −0.672 |
| FLJ22104 | 0.2365 | −0.1955 | −0.2125 | −0.8255 | −0.7525 | 0.4185 | −0.9545 | 0.5475 | 0.4745 | −0.3175 | −0.8195 | 0.3185 | −0.0805 |
| FLJ22222 | −0.209 | 0.379 | 0.381 | 0.265 | 0.35 | −0.035 | −0.83 | −0.15 | −0.099 | 0.095 | 0.119 | 0.539 | 0.041 |
| FLJ22573 | 1.5835 | 0.3055 | 0.9755 | 0.7655 | −0.5705 | −0.5325 | 1.7535 | −0.0145 | 0.0795 | 1.8455 | 0.3665 | 0.6145 | −0.4185 |
| FLJ22794 | 0.096 | 0.336 | −0.313 | 0.117 | −0.066 | −0.005 | −0.26 | 0.347 | 0.382 | 0.26 | 0.115 | −0.334 | −0.316 |
| FLJ23188 | 1.3405 | −0.4905 | −0.7215 | −0.0505 | 0.2335 | −0.0995 | −0.1345 | −0.1175 | −0.0935 | 0.5225 | −0.2685 | 5.2925 | −0.0685 |
| FLJ23441 | 0.477 | 0.042 | 0.017 | 0.226 | 0.76 | 0.57 | 0.772 | 0.048 | 0.213 | 1.05 | 0.331 | 0.713 | 1.399 |
| FLJ25471 | 1.157 | 0.381 | −0.122 | −0.023 | −0.168 | 0.445 | −0.133 | 0.97 | 0.958 | 0.069 | 0.273 | 0.473 | 0.266 |
| FLJ31204 | −0.9125 | −1.7815 | 0.2145 | 2.0445 | 0.0905 | 0.1025 | 0.0735 | −2.1525 | −1.4885 | −1.2115 | −0.0105 | −0.9115 | −0.4595 |
| FLJ31795 | −0.5595 | −0.0515 | −0.8045 | 0.1595 | −0.0105 | 0.0355 | 0.6985 | 0.3515 | 0.2655 | −0.4205 | −0.0365 | 0.6025 | −0.5545 |
| FLJ32942 | −0.0805 | 1.7635 | −0.0065 | 0.7335 | −0.3825 | 0.0625 | 0.0685 | 0.4275 | 0.4355 | 1.1995 | −0.9665 | 1.5475 | 1.5495 |
| FLJ37970 | 1.837 | 1.963 | −0.213 | 0.128 | −0.004 | −0.071 | 0.438 | −0.621 | −0.745 | −1.271 | 1.053 | −1.014 | 0.527 |
| FLJ39370 | −0.1265 | −0.0165 | −0.2565 | −0.2145 | 0.2105 | −0.1205 | 0.2865 | 1.0955 | 1.0665 | 0.1665 | 0.6825 | 0.3315 | −0.1385 |
| FLJ90588 | −0.8405 | −1.9545 | −0.0225 | −0.3135 | −0.3475 | −0.4155 | −0.5315 | 4.7875 | 4.9035 | −1.9665 | 0.0175 | −0.7165 | −1.0325 |
| FN3KRP | 0.622 | 0.464 | 0.893 | 0.424 | 0.614 | 0.644 | 0.48 | 0.394 | 0.394 | 1.054 | 0.704 | 0.833 | 1.073 |
| FOLR1 | −1.0995 | −0.7005 | −0.0395 | −0.7555 | 0.0045 | −0.1235 | 0.8005 | −1.8825 | −1.7335 | −1.3725 | −0.3825 | 2.4005 | −1.0895 |
| FOXA1 | 0.2145 | −0.5285 | 0.0195 | 0.4525 | 0.0585 | 0.4325 | −0.0805 | −0.1775 | −0.2505 | 0.0045 | −1.1355 | 0.8826 | 0.1255 |
| FOXC1 | 0.138 | −0.002 | −0.637 | 0.543 | −0.085 | 0.171 | −0.871 | −0.248 | −0.169 | −0.027 | −0.448 | −2.324 | −1.096 |
| FOXP1 | −0.3305 | −0.4966 | −0.2525 | 1.0105 | 1.4725 | −0.1275 | −0.0175 | 0.8415 | 0.8895 | −0.5485 | 0.0705 | 1.7065 | 0.6685 |
| FREQ | 0.2595 | 0.1745 | 0.4375 | 0.3855 | 0.0295 | 0.3355 | 0.0355 | −0.3625 | −0.3045 | 0.1155 | −0.3515 | 0.0005 | 0.1375 |
| FTO | 0.2725 | 0.6995 | 0.2575 | 0.9755 | 0.2685 | −0.1015 | 0.8385 | 0.1335 | 0.2015 | 0.8585 | 0.2755 | 0.2455 | 0.6875 |
| FUBP1 | 0.9615 | 1.2415 | 0.8605 | 0.8395 | 0.2495 | 0.6625 | 1.0685 | −0.0025 | −0.0495 | 1.2585 | −0.0645 | 0.5945 | 1.5535 |
| FUCA1 | 0.404 | −0.519 | −0.54 | 0.147 | 0.097 | 0.177 | −0.249 | 0.003 | −0.216 | −0.133 | −0.404 | −0.881 | −0.855 |
| FUT8 | 1.4605 | 0.5295 | −0.2185 | 0.2335 | 0.1825 | 0.2525 | 0.3605 | −0.4485 | −0.4455 | −0.0065 | 0.1775 | 0.2555 | 0.2595 |
| FXYD6 | 1.272 | 0.697 | 0.297 | −0.049 | 0.223 | 0.953 | −0.531 | −0.464 | −0.501 | −0.163 | −0.419 | −1.237 | 0.041 |
| FYCO1 | −0.079 | −0.502 | 0.571 | 0.702 | −0.415 | 0.868 | −1.213 | −0.359 | −0.368 | −0.203 | −0.423 | −1.397 | 0.822 |
| FZD10 | −0.0465 | 0.3455 | −0.2585 | 0.0995 | 0.9876 | −0.2335 | −0.2775 | −0.9275 | −0.4105 | −0.4265 | −0.0505 | 0.0905 | −0.3215 |
| FZD7 | −0.4605 | −1.4935 | −1.3855 | −0.7335 | −0.0645 | 0.2465 | 0.1955 | 2.1055 | 2.0755 | 1.2685 | 2.7905 | 0.1175 | −0.1235 |
| FZD9 | −0.7975 | −0.5625 | −0.7575 | −0.5155 | 0.4565 | 0.6805 | −0.1735 | 3.1865 | 2.8355 | 0.9465 | 2.9755 | 0.1665 | 1.0035 |
| G1P2 | 0.4055 | 0.1265 | 0.3065 | 0.1185 | 0.2895 | 0.0115 | 0.5885 | 0.2255 | 0.3595 | 0.5385 | −0.1045 | −0.3165 | −0.1545 |
| G1P3 | 0.903 | 0.292 | 0.363 | −0.049 | 0.009 | −0.213 | 1.052 | 1.344 | 1.303 | 0.5 | 0.897 | −0.491 | 0.059 |
| GA17 | −0.7905 | −1.9176 | −1.1915 | −0.5705 | −0.8485 | 0.0095 | −3.0485 | 0.2855 | 0.2425 | 0.1705 | 0.2855 | −1.0785 | −0.2685 |
| GABARAP | −1.0875 | −1.0135 | −0.9035 | −0.3545 | −0.4715 | 0.0745 | −0.9235 | −0.7795 | −0.6155 | −0.0395 | −0.2885 | 0.2235 | 0.2055 |
| GAJ | −1.7085 | 0.0845 | −0.1905 | −1.0645 | −0.8895 | 0.3185 | 0.8945 | −0.2885 | −0.3585 | −0.9955 | 1.3425 | −0.6035 | −0.5875 |
| GALE | −0.459 | −1.313 | −0.49 | 0.048 | 0.044 | −0.037 | −0.302 | −0.344 | −0.331 | −0.55 | −0.634 | −0.878 | −0.948 |
| GALNT14 | 0.3115 | 1.1475 | 0.5535 | 0.1635 | −0.7885 | 0.1485 | 1.1725 | −0.0105 | −0.0455 | 0.7475 | 0.2805 | 1.0035 | 1.2635 |
| GALNT2 | 0.0295 | −0.7955 | 0.3145 | −0.0735 | 0.5255 | 0.3265 | 0.8115 | −0.0505 | −0.0585 | 0.8325 | 0.2525 | 1.5545 | 0.2685 |
| GALNT7 | −0.772 | −0.971 | −1.123 | −0.528 | 0.299 | −0.442 | −0.945 | −0.224 | 0.006 | 0.117 | 0.357 | −0.924 | −1.034 |
| GAMT | 0.243 | −0.422 | −0.567 | 0.134 | 0.001 | −0.026 | −0.034 | 0.171 | 0.188 | −0.364 | −0.337 | −0.33 | −1.321 |
| GAPD | −0.573 | 0.353 | −0.65 | −0.071 | −0.224 | −0.179 | −0.331 | 0.094 | 0.139 | 0.09 | −0.994 | −0.169 | −0.553 |
| GARS | 0.267 | −0.366 | 0.225 | 0.694 | 1.264 | 0.641 | 2.25 | 0.816 | 0.883 | 1.202 | 0.61 | 0.646 | 1.4 |
| GART | 0.6615 | 1.3505 | 0.6116 | −0.3425 | 0.1945 | 0.6075 | 1.5535 | 0.6355 | 0.7375 | 1.1065 | 0.7055 | −0.0405 | −0.2985 |
| GATA3 | −0.1865 | −0.5085 | −0.4805 | −0.3645 | 0.0055 | −0.4365 | 0.7715 | −0.4585 | −0.3675 | 0.2955 | 0.1525 | 0.2515 | 0.7265 |
| GATM | 0.066 | 0.63 | −0.217 | −0.613 | 0.885 | 1.421 | 1.889 | 4.852 | 4.67 | 0.175 | 0.564 | −0.319 | 2.024 |
| GBA | −0.22 | −0.034 | −0.53 | −0.098 | −0.095 | −0.188 | −0.074 | 0.573 | 0.59 | 0.523 | 0.058 | −0.229 | −0.411 |
| GDAP1 | −0.624 | −0.104 | −0.302 | 0.257 | −0.007 | 0.068 | 0.705 | −0.378 | −0.333 | −0.224 | −0.333 | 0.476 | 0.106 |
| GDI2 | −0.7605 | 0.0755 | 0.5045 | 0.1515 | −0.1055 | −0.0065 | 0.1675 | 0.3765 | 0.5025 | 0.3255 | 0.3565 | 1.0205 | 0.2245 |
| GGA1 | 0.2725 | 0.0045 | 0.9955 | 0.6595 | 0.5665 | 0.6435 | −0.8855 | −0.1315 | −0.5605 | 0.4355 | −1.3685 | 0.8485 | 0.6745 |
| GKAP1 | 0.4405 | −0.6365 | −0.4925 | 0.0725 | 0.1145 | −0.2835 | −0.2865 | −0.4005 | −0.5175 | −0.2665 | −0.1895 | −0.4085 | −0.6735 |
| GLCCI1 | −0.1976 | 0.0025 | 0.5075 | 0.0715 | 0.5905 | −0.0085 | 0.0335 | 0.2455 | 0.1165 | 0.4385 | −0.8395 | 0.1655 | 0.0455 |
| GLG1 | 0.497 | 0.097 | −0.028 | −0.353 | 0.867 | 0.097 | −0.395 | 0.649 | 0.711 | −0.022 | 0.244 | −1.459 | 0.887 |
| GLTSCR2 | 0.585 | 1.859 | 0.639 | −0.128 | 0.864 | 0.581 | −0.137 | −0.494 | −0.374 | 0.029 | −0.425 | 0.457 | 0.586 |
| GNB4 | 0.11 | −0.225 | 0.203 | −0.137 | 0.064 | −0.138 | −0.233 | −0.519 | −0.491 | −0.538 | −0.086 | 0.298 | 0.692 |
| GOLPH2 | −0.2075 | −0.1005 | −0.3385 | 0.1805 | −0.2285 | 0.1305 | −0.2515 | −0.7975 | −0.8475 | −1.1835 | −0.0245 | −0.1495 | −0.6395 |
| GPATC1 | 0.1295 | 0.1555 | 0.2865 | 0.2225 | −0.6565 | 0.3905 | 0.2015 | −0.8845 | −0.9275 | −0.5125 | 0.1855 | −0.1215 | −0.5715 |
| GPC2 | 0.2175 | 1.2625 | 0.9375 | −0.7115 | −0.8895 | −0.6365 | 1.1285 | 1.5335 | 1.4535 | 1.4525 | 0.3055 | 1.2385 | 1.0665 |
| GPR125 | −1.124 | −1.302 | −1.716 | −1.643 | −0.995 | −0.415 | 0.038 | −0.842 | −0.731 | −0.428 | −0.011 | −0.806 | −0.165 |
| GPR160 | 0.5715 | 0.2655 | 0.2325 | −0.2965 | −0.3845 | −0.4475 | 0.4805 | 0.7405 | 0.7475 | −0.2195 | 0.7845 | 0.2155 | 0.4195 |
| GPR56 | 0.1965 | −0.1845 | 0.0935 | 0.4065 | 0.3485 | 0.6485 | −0.1735 | 0.0185 | 0.0265 | −0.1495 | 0.1175 | −1.1395 | −0.3845 |
| GPRC5C | 0.0875 | 0.7845 | −0.7625 | −0.3325 | −0.4225 | −0.7395 | −1.4835 | −0.2365 | −0.2055 | −0.7035 | −0.7135 | 0.5155 | 0.0945 |
| GPSM1 | −0.549 | 0.345 | −1.112 | 0.677 | −0.084 | 0.211 | −0.37 | 0.761 | 0.836 | 0.242 | 0.347 | 0.084 | −0.321 |
| GPSM2 | −0.908 | 0.71 | −0.993 | −0.704 | −1.594 | −0.287 | −1.088 | −0.262 | −0.21 | −0.674 | −0.428 | −0.86 | −0.899 |
| GPT | 1.7625 | 0.0835 | 0.7795 | 1.5515 | 1.0885 | 0.6305 | −0.8525 | −0.0465 | 0.0695 | 0.5365 | 0.3595 | −1.6195 | −1.0285 |
| GPT2 | −1.47 | 0.008 | −0.467 | −1.53 | −1.351 | 0.588 | −0.022 | −0.046 | 0.053 | −1.408 | −0.748 | −2.458 | −1.326 |
| GPX7 | −0.003 | −0.526 | −0.408 | −0.244 | 0.005 | 0.069 | −0.074 | 0.225 | −0.007 | −0.444 | −0.148 | 0.385 | 0.079 |
| GRB14 | 0.8675 | 0.7535 | 0.0775 | −0.2885 | 0.0265 | −0.7635 | 0.9565 | 1.0575 | 1.1935 | 0.6805 | −1.2475 | −1.3935 | 2.3115 |
| GRB7 | −0.3845 | 0.2955 | −0.1955 | −1.0565 | −0.7805 | −0.1755 | −0.3675 | 0.6395 | 0.4725 | −0.5945 | 0.3285 | 0.0955 | −0.5855 |
| GSC | −0.426 | −0.552 | −0.257 | 0.246 | 0.11 | 0.184 | 0.057 | 0.037 | 0.142 | −0.104 | −0.302 | −0.981 | −0.407 |
| GSDML | 0.203 | 0.142 | 0.111 | −0.225 | −0.41 | −0.119 | −0.486 | −0.12 | −0.405 | −0.679 | 0.081 | 0.506 | −0.299 |
| GSK3B | 0.766 | 0.091 | 0.021 | −0.017 | −0.289 | −0.202 | 0.685 | 0.929 | 0.855 | −0.099 | 1.068 | 0.215 | 0.275 |
| GSTA1 | 1.4215 | 2.9525 | 2.9235 | 1.0055 | 0.9125 | −1.1205 | 0.6285 | 1.6015 | 1.8876 | 0.9305 | −1.8885 | 2.2625 | 2.3135 |
| GSTK1 | −0.778 | 0.849 | −1.728 | −0.228 | −0.779 | −0.718 | −1.359 | −1.227 | −1.224 | −0.652 | −1.775 | −1.121 | −1.062 |
| GSTM3 | −1.3265 | −2.5365 | 0.1675 | 0.0825 | 0.4205 | −0.2755 | −2.0945 | 1.2895 | 1.1805 | −0.4185 | 0.3215 | 0.1695 | 0.8565 |
| GSTP1 | −0.024 | 1.234 | 0.98 | 0.458 | 0.714 | 1.14 | −0.124 | −0.398 | −0.099 | −0.808 | −1.002 | −0.793 | 0.628 |
| GSTT1 | 0.7815 | 0.3155 | −0.2755 | 0.2465 | 0.6155 | 0.3675 | 0.0576 | −0.1205 | −0.1275 | 0.4805 | 0.4455 | −0.8535 | 0.1675 |
| GSTT2 | 0.2765 | 0.1145 | 0.7755 | −0.1085 | 0.1755 | −0.1325 | 0.2565 | 0.3345 | 0.3375 | −0.0275 | 0.5925 | 0.4655 | 1.0885 |
| GTF2F2 | −0.18 | 0.191 | −0.773 | −0.188 | 0.239 | −0.057 | −0.005 | −0.108 | −0.048 | 0.217 | 0.718 | −0.64 | 0.337 |
| GTF2I | 0.2645 | 0.9595 | −0.1835 | 0.2275 | 0.1025 | −0.0275 | 0.1536 | 0.2665 | 0.1475 | −0.4015 | −0.1305 | −0.4645 | 0.2425 |
| GTF3A | 0.057 | −0.006 | −0.019 | −0.229 | −0.831 | −0.128 | −0.137 | −0.021 | −0.011 | −0.381 | −0.038 | 0.258 | 0.019 |
| GTPBP3 | −0.251 | −0.771 | −0.891 | 0.703 | 0.32 | 0.821 | −0.597 | −1.288 | −1.207 | 0.151 | −0.387 | 0.47 | −0.114 |
| GTPBP4 | 0.24 | −0.282 | 0.265 | −0.472 | −0.264 | −0.178 | −0.006 | 0.045 | 0.145 | 0 | −0.489 | −0.75 | −0.26 |
| H1F0 | −0.4335 | −0.8025 | −0.1445 | −0.2745 | 0.4465 | −0.1215 | 0.1505 | 0.3715 | 0.4235 | −0.2465 | 0.1525 | 0.4625 | −0.3845 |
| H2AFV | −0.1235 | −0.1595 | −0.3855 | −0.6285 | −0.7335 | −0.7285 | −0.9385 | 0.6345 | 0.6005 | 0.0185 | −0.0955 | 0.0345 | −0.3785 |
| H2AFY | 0.4925 | −0.4345 | 0.4885 | −0.9545 | −0.5265 | −0.5225 | 2.0125 | 1.7235 | 1.9735 | 1.3145 | 0.4695 | −0.4065 | −0.6895 |
| H2AFZ | 0.8875 | 0.1995 | −0.2535 | −0.6375 | −0.3785 | −0.3235 | −0.0525 | 1.0735 | 1.2265 | 0.0875 | 0.7285 | −0.9695 | −0.3165 |
| H2BFS | 0.1515 | −0.2585 | 0.1095 | −0.0755 | 0.0165 | 0.1305 | −0.4635 | 0.8865 | 0.7965 | −0.0375 | −0.2675 | −0.1785 | −0.4135 |
| H3F3B | 0.249 | −0.132 | −0.477 | −0.357 | 0.306 | −0.433 | 0.647 | 0.562 | 0.655 | 0.068 | 0.584 | 1.731 | 1.142 |
| H41 | −0.3375 | −3.3965 | −0.2455 | 0.7545 | 0.6855 | 0.0745 | 0.2555 | −0.7125 | −1.0545 | −0.9595 | −0.0725 | −0.9445 | −0.4085 |
| HAGH | 0.539 | 0.218 | 0.367 | −0.407 | −0.12 | 0.039 | 0.841 | −0.219 | −0.073 | 0.636 | −0.875 | −0.32 | 0.02 |
| HBG1 | −1.2585 | −2.7355 | −1.0105 | −1.3125 | −0.8165 | −0.2755 | −2.8285 | −0.4085 | −0.2435 | −0.7495 | −0.2685 | −0.3085 | −0.0855 |
| HBL01 | −0.41 | −1.187 | −0.646 | −0.394 | −0.385 | −0.145 | −0.649 | 1.68 | 1.492 | 0.937 | 0.674 | 0.538 | −0.162 |
| HCAP-G | −0.5485 | 0.2755 | −0.4095 | 0.0475 | 0.1265 | 0.2515 | −0.9895 | −0.5495 | −0.6495 | −0.2975 | −0.1485 | −0.3315 | 0.1245 |
| HCP5 | −1.006 | 0.953 | 0.048 | 0.706 | 0.232 | 1.793 | 0.987 | 0.344 | 0.14 | 0.532 | −0.841 | 0.442 | 3.969 |
| HDAC2 | −0.309 | 0.494 | 0.423 | 0.422 | 0.505 | 0.019 | 3.818 | 0.382 | 0.492 | 1.305 | −0.071 | 0.656 | 0.401 |
| HDC | −0.1175 | −0.0585 | −0.0385 | −0.2745 | −0.3365 | −0.1915 | 0.2125 | −0.2315 | −0.4275 | −0.4275 | −0.0055 | 0.8225 | 0.5185 |
| HEBP1 | 0.202 | 0.235 | 0.686 | 1.001 | −0.077 | 0.855 | 0.177 | 1.218 | 1.046 | −0.902 | −0.761 | 0.825 | 0.265 |
| HIPK2 | 1.762 | 1.258 | 1.06 | 1.008 | 0.672 | 0.794 | 0.758 | 1.409 | 1.329 | 0.492 | 0.181 | −0.384 | 0.293 |
| HIPK2 | 0.2125 | −0.6265 | 1.4195 | −0.9145 | −0.9815 | −0.7895 | 3.7245 | 1.1425 | 1.2045 | 1.2115 | 0.5545 | −0.1575 | −0.3825 |
| HIS1 | 0.2905 | −0.5465 | 0.7655 | −0.8455 | −1.1015 | −0.8725 | 2.4295 | 1.6615 | 1.7755 | 1.2705 | 0.1225 | −0.4645 | −0.6075 |
| HIST1H2A | 0.253 | −0.581 | 0.424 | −0.795 | −1.055 | −0.759 | 2.448 | 1.761 | 1.755 | 1.144 | 0.042 | −0.306 | −0.743 |
| HIST1H2B | 0.1255 | −0.6055 | 0.6105 | −0.9505 | −1.1795 | −1.0595 | 2.2085 | 1.7015 | 1.6285 | 1.1205 | 0.1115 | −1.2105 | −0.8295 |
| HIST1H2B | 0.168 | −0.354 | 0.675 | −0.698 | −1.138 | −0.875 | 2.359 | 1.837 | 1.703 | 1.18 | 0.181 | −0.367 | −0.626 |
| HIST1H2B | 0.4805 | 0.1305 | −0.3955 | −0.3585 | −0.3665 | −0.2305 | −0.0225 | 1.1745 | 1.0535 | −0.0335 | 0.7715 | −0.5995 | −0.1475 |
| HIST1H2B | 0.2985 | −0.2925 | −0.1385 | −0.3095 | −0.1025 | −0.2425 | 0.1605 | −0.1065 | −0.0615 | 0.2065 | 0.0115 | −0.1295 | −0.3265 |
| HIST1H3C | −0.6135 | 0.4915 | −1.0575 | −0.6145 | −0.5595 | −0.5885 | −0.5935 | −0.4745 | −0.2785 | −0.8165 | −0.6735 | 1.5485 | −0.9575 |
| HM13 | 0.2895 | −1.5895 | −0.8525 | −0.7605 | −0.8135 | −0.5935 | −1.0985 | −0.2765 | −0.1445 | −0.5845 | −0.0005 | −0.5875 | −1.8145 |
| HMGB3 | −0.4395 | −0.5155 | −0.0275 | −0.0925 | 0.0245 | 0.2065 | −0.2895 | −0.2425 | −0.2425 | −0.2875 | −0.2885 | −0.1385 | 0.3615 |
| HN1 | −0.216 | −0.141 | 0.216 | −0.394 | 0.061 | −0.168 | −0.562 | 0.141 | 0.136 | 0.268 | 0.022 | −0.171 | −0.089 |
| HNRPA3P | 1.6815 | 0.3955 | 0.1735 | 0.8365 | 0.3175 | 0.4555 | −0.2245 | 0.3795 | 0.1015 | −0.4805 | −0.4085 | 1.6035 | −0.5655 |
| HNRPDL | 3.28 | 0.829 | 0.301 | 0.868 | 0.739 | 0.682 | 0.342 | −0.333 | −0.41 | 0.009 | −0.875 | 0.474 | −0.824 |
| HOXA5 | 0.3185 | −0.9065 | −1.1755 | 1.3735 | −1.0055 | 0.9955 | 0.5065 | −0.8385 | −0.5815 | 0.1035 | −1.2075 | 2.7315 | −2.3905 |
| HOXA7 | 0.7455 | 0.2935 | 0.4875 | 2.1415 | 0.1595 | 0.1695 | −0.0215 | 0.9295 | 0.8905 | 1.0695 | 0.2065 | 4.6545 | −1.5725 |
| HOXB2 | 1.0105 | −0.0215 | −1.2215 | 0.4195 | −0.1885 | −0.4945 | −0.1585 | 0.8345 | 0.8595 | 0.1005 | −1.0395 | 1.2985 | −1.3885 |
| HOXB6 | −1.9975 | −1.0505 | −1.2855 | 0.2235 | 1.5125 | 0.6335 | −0.0685 | 1.0315 | 0.6015 | −0.6245 | −1.2435 | 0.6805 | 1.1075 |
| HOXB7 | 0.3525 | −0.0575 | −1.2885 | −0.5925 | 1.9935 | −0.0155 | −0.4555 | 0.5085 | 0.4915 | 0.4415 | −0.5875 | −0.5325 | 1.2075 |
| HOXC10 | 0.832 | 0.02 | −0.054 | 0.274 | 0.994 | 0.247 | 1.178 | 0.443 | 0.45 | 0.567 | 0.498 | −0.61 | 0.207 |
| HOXC13 | −0.2755 | 0.2045 | −1.5185 | 0.6825 | 0.0745 | 0.3135 | −0.2995 | 0.5285 | 0.4185 | 0.4325 | 0.0185 | −1.0445 | −0.8945 |
| HOXC9 | −0.146 | 0.554 | 0.677 | 0.502 | 0.635 | 0.28 | −0.147 | −0.297 | −0.222 | 0.507 | −0.215 | −0.966 | −0.01 |
| HOXD3 | 0.5895 | 0.0285 | 0.1185 | −0.2015 | −0.3016 | 0.1385 | 0.5185 | 0.4885 | 0.4285 | 0.1885 | −0.1715 | −0.1515 | 0.4775 |
| HPCL2 | −0.564 | −0.87 | −0.167 | −0.431 | −0.22 | −0.121 | 0.727 | 1.145 | 1.063 | 0.340 | 0.938 | −1.879 | −1.701 |
| HPS3 | −0.2635 | 0.1005 | −0.7685 | 0.0645 | −0.3185 | 0.0865 | −0.0385 | −0.0245 | 0.0975 | 0.3765 | −0.0015 | −1.1095 | 0.0995 |
| HRASLS | −0.2455 | 0.7995 | −0.1135 | −0.5755 | −0.3825 | 0.4875 | 1.0065 | 1.3765 | 1.3805 | 1.1165 | −0.1555 | 0.6375 | 0.2985 |
| HRB | −1.238 | −0.946 | −1.178 | −0.025 | −0.747 | 0.368 | −0.44 | −2.489 | −2.445 | −0.369 | −0.518 | −1.246 | −1.514 |
| HRSP12 | 0.032 | 0.19 | −0.12 | −0.201 | 0.091 | 0.495 | −0.434 | 0.14 | 0.132 | −0.184 | 0.221 | 0.355 | 0.668 |
| HSD17B2 | −0.6355 | −0.1185 | −0.1915 | −0.2745 | −0.5225 | −0.0945 | −0.0955 | −0.1785 | −0.1685 | 0.2605 | −0.2415 | −0.6935 | −0.2155 |
| HSF1 | 0.9485 | 1.1775 | 0.7095 | 0.7905 | 0.9355 | 0.3925 | −0.0345 | −0.8555 | −0.9635 | −0.9505 | −0.3955 | −1.5525 | 1.4975 |
| HSPA14 | 0.774 | −0.633 | 0.347 | −1.172 | −0.836 | −0.44 | −0.989 | 0.657 | 0.65 | 0.258 | −0.226 | −0.549 | −1.07 |
| HSPA2 | 0.4885 | 0.1595 | −0.3215 | 0.1905 | 0.1745 | 0.0405 | 0.7585 | −0.2465 | −0.2135 | −0.0815 | −0.2375 | 0.1165 | 0.4505 |
| HSPA5BP [symbol] | 0.671 | −0.211 | 0.001 | 0.091 | 0.079 | −0.298 | −1.713 | 0.135 | 0.18 | −0.186 | −0.147 | −1.316 | −0.871 |
| HSPC016 | 0.839 | 0.158 | −0.087 | −0.037 | −0.024 | −0.487 | −0.85 | −0.368 | −0.343 | −0.183 | −0.444 | −0.824 | −0.772 |
| HSPC138 | −1.394 | −1.515 | −0.251 | −0.845 | −1.215 | −0.41 | −2.391 | −0.854 | −0.559 | −0.422 | −1.158 | −0.796 | −0.588 |
| HSPC148 | 0.0245 | −0.4805 | −0.0276 | −0.7135 | −0.4155 | 0.0225 | 0.1575 | 0.7995 | 0.6955 | 0.2005 | −0.5965 | 8.3575 | −0.8065 |
| HSPC150 | −0.0945 | 0.4795 | −0.0485 | −0.1135 | −0.3615 | −0.3435 | −0.0325 | 1.1605 | 1.1235 | 0.4165 | 0.4155 | −0.4795 | −0.2335 |
| HSPC163 | 0.165 | 0.185 | 0.057 | −0.083 | 0.137 | 0.387 | −0.638 | 0.27 | 0.383 | −0.223 | −0.331 | −0.573 | −1.355 |
| HSPC266 | −0.408 | 0.05 | −1.18 | −0.483 | −1.094 | 0.004 | 1.4 | 1.182 | 1.113 | 0.351 | 1.198 | 0.029 | −0.647 |
| HSU79274 | 0.606 | −0.219 | −0.063 | 0.124 | −0.096 | 0.208 | −0.692 | −0.483 | −0.468 | −0.223 | 0.032 | −1.009 | −1.147 |
| HTATIP2 | 0.207 | 0.3 | 0.076 | −0.57 | −0.148 | −0.259 | 0.016 | 0.462 | 0.455 | −0.169 | 0.696 | −0.276 | 8.063 |
| ICMT | −0.743 | −0.043 | 0.103 | −0.733 | −1.089 | 0.043 | −1.157 | −1.009 | −1.094 | −1.708 | −1.299 | −0.373 | 1.744 |
| ICT1 | −0.598 | −0.612 | −0.174 | −0.293 | −0.78 | −0.53 | −0.382 | −0.018 | 0.061 | −0.293 | 0.057 | −0.534 | −0.701 |
| ID4 | 1.0375 | 0.0505 | 0.0595 | 0.6785 | 0.5795 | 0.5485 | 0.2885 | 1.0085 | 1.0505 | 0.5215 | 1.4335 | −0.2015 | 0.2415 |
| IDH2 | 0.043 | −0.396 | −0.906 | −0.293 | −0.007 | −0.255 | −0.546 | 0.079 | 0.191 | 0.096 | −0.462 | 0.725 | −0.743 |
| IDS | −0.5935 | −0.0175 | −0.5445 | −0.4015 | −0.2905 | 0.3305 | 0.8015 | 2.0055 | 1.7775 | 0.5765 | 2.0155 | −0.5045 | −0.4195 |
| IFI30 | −0.2095 | −0.8205 | 0.0215 | 0.1975 | −0.3435 | 0.3415 | 0.1305 | 1.6565 | 1.7325 | 0.5945 | 0.8555 | −0.1775 | −0.1105 |
| IFI44 | −1.4085 | −0.7365 | −0.7105 | 0.2155 | 1.6375 | 0.4585 | 1.6955 | 0.9445 | 0.7235 | 0.6335 | 1.3035 | −0.0095 | 0.1945 |
| IFIT5 | 0.4375 | −0.2375 | 0.3885 | −0.1005 | −0.1385 | 0.0095 | 0.3575 | 0.5785 | 0.6795 | 0.5415 | −0.3435 | 0.5035 | 0.0885 |
| IFITM1 | 0.0685 | −0.2505 | 0.1115 | 1.4065 | 2.8785 | −0.0585 | 0.8925 | 1.0595 | 0.9075 | 2.5755 | −0.1525 | 0.3275 | 2.4945 |
| IGBP1 | 0.6205 | −0.3415 | 0.8245 | −0.3975 | 0.0115 | −0.6585 | −2.3175 | −0.4775 | −0.4625 | 0.0575 | −1.5395 | 0.8615 | −0.3845 |
| IGFALS | −0.1495 | −0.1065 | −0.2805 | 0.2635 | −0.6385 | −0.4065 | 0.0005 | 0.8625 | 0.6915 | 0.0035 | −0.6915 | 2.3005 | 2.2655 |
| IGPBP2 | −0.8025 | −0.0945 | −0.3935 | −0.4245 | −0.3665 | −0.3065 | −0.1135 | 1.2895 | 1.5625 | 0.0005 | −0.3165 | 1.8655 | 2.5025 |
| IGHG1 | −3.7635 | 0.6295 | 1.6445 | 0.7285 | −1.1945 | 0.5915 | −0.8765 | −1.0285 | −0.8465 | −1.8565 | −0.8875 | 0.9345 | 0.3085 |
| IGHG1 | −1.868 | −1.116 | −0.58 | −0.226 | −1.414 | −0.495 | −1.12 | 1.311 | 1.449 | −0.31 | −0.98 | 3.083 | 1.58 |
| IGJ | −1.872 | −1.409 | −0.963 | −0.721 | −1.723 | −1.329 | −1.835 | 0.582 | 0.622 | −0.955 | −1.096 | 0.844 | 1.948 |
| IGKC | −0.863 | −0.537 | −0.582 | 0.066 | 0.04 | −0.326 | −0.654 | 1.887 | 2.209 | −0.257 | −0.604 | 0.571 | 1.28 |
| IGKC | −1.99 | −0.903 | −0.957 | −0.033 | −0.878 | −0.791 | −1.276 | 1.76 | 1.692 | −0.882 | −1.128 | 1.209 | 2.062 |
| IGLC2 | 0.4285 | 0.2605 | −0.0755 | 0.6855 | 0.2305 | 0.3895 | −0.3065 | 0.1025 | 0.1435 | 0.7515 | −0.2145 | −0.6675 | −0.6285 |
| IGLL1 | −0.3765 | 0.2095 | −0.3165 | −0.0525 | −0.2105 | 1.1135 | −0.3025 | 0.3235 | 8.1085 | −0.1415 | −0.0585 | −0.4085 | 1.2885 |
| IL13RA1 | −0.479 | 0.346 | −0.345 | −0.047 | 0.006 | 0.132 | 1.591 | 0.746 | 0.818 | 0.024 | −0.181 | 0.08 | 0.867 |
| IL17B | 0.0435 | 2.1745 | 1.6255 | 1.7495 | 2.2145 | 1.4025 | −0.8995 | 2.0335 | 2.0065 | 1.3985 | 0.0145 | −0.0455 | 1.5245 |
| IL23A | −0.4855 | −1.2155 | 0.2775 | −0.3025 | −0.0035 | −0.1745 | −0.3955 | −0.8615 | −0.9175 | −0.3985 | 0.0725 | −0.1375 | 1.3135 |
| IL6ST | −1.2695 | −0.3225 | −0.7375 | −0.2415 | −0.6305 | 0.3955 | −1.1065 | −0.7125 | −0.7215 | −0.1915 | −0.6025 | 0.2225 | −0.3145 |
| ILF2 | 0.962 | 0.241 | 1.139 | 0.352 | 0.066 | −0.377 | −0.428 | 0.483 | 0.503 | 0.89 | 0.224 | −0.6 | 0.16 |
| IMPA2 | 0.9695 | −2.3065 | 0.4975 | 0.5265 | −0.1235 | −0.9805 | 0.4045 | −0.9975 | −1.0295 | −0.7265 | −0.5355 | −0.7425 | −0.9415 |
| ING1L | 2.729 | 1.311 | 1.607 | 2.121 | 0.659 | 1.703 | 0.704 | 0.406 | 0.425 | 0.631 | 0.643 | 1.122 | 8.61 |
| INHBB | 1.4145 | −0.2145 | −0.4825 | −0.1495 | 0.7435 | 0.6485 | 0.1315 | 5.5585 | 5.6415 | −0.1905 | 0.5785 | −0.2225 | 3.2295 |
| INPP4B | 2.4175 | −1.1305 | −1.2025 | 0.8415 | 0.5145 | 1.4225 | −0.4335 | 2.8135 | 2.5595 | 1.0715 | −0.9705 | −1.4345 | −1.1705 |
| INSM1 | 0.634 | −0.821 | 0.395 | 0.663 | 0.505 | 0.181 | 0.099 | 0.104 | 0.213 | 0.29 | 0.793 | 0.444 | 0.664 |
| IQCA | −0.347 | 0.489 | 1.19 | 1.155 | 0.123 | 0.439 | −1.772 | 0.189 | 0.285 | 1.275 | −0.225 | 2.051 | −0.893 |
| IQCE | −0.855 | −0.943 | −0.054 | 0.342 | 0.022 | 0.042 | −0.391 | 0.374 | 0.479 | −0.596 | −0.422 | 1.131 | −0.067 |
| IQGAP2 | −0.8525 | −1.1805 | −0.4565 | 0.1655 | −1.0995 | −0.2885 | 0.4815 | 1.1475 | 1.1675 | −0.6005 | −0.6405 | 0.9475 | 0.9435 |
| IRF4 | −0.9395 | −0.2405 | −0.7085 | −1.0805 | 0.0015 | 1.2195 | 1.5665 | 2.1085 | 2.0675 | −2.9125 | −1.0575 | −1.9745 | 0.7575 |
| IRTA2 | −0.091 | −0.09 | −0.822 | −0.961 | −0.341 | 0.175 | −0.278 | 1.645 | 1.575 | 0.128 | 1.052 | −0.081 | 0.91 |
| IRX4 | −0.5325 | −1.1255 | −0.8515 | −0.3715 | −0.0415 | 0.1445 | 0.2305 | 1.4315 | 1.3505 | 0.3965 | 1.2185 | 0.1275 | 0.3665 |
| ISG20 | 0.802 | −0.536 | −1.141 | −0.431 | −0.802 | 0.375 | −0.319 | −1.75 | −1.774 | −0.815 | −0.268 | 0.036 | 1.32 |
| ISGF3 [symbol] | −1.135 | 1.353 | 1.979 | 1.296 | 0.484 | 0.463 | 1.96 | 0.087 | 0.129 | 0.515 | 0.313 | 0.858 | 0.742 |
| ITGB4 | −0.084 | −0.445 | 0.057 | −0.084 | −0.078 | 0.206 | 0.183 | −0.058 | −0.046 | −0.449 | −0.033 | 0.129 | −0.81 |
| ITPR1 | 0.241 | −1.049 | 0.824 | 0.346 | 1.11 | 0.489 | 1.573 | 0.563 | 0.568 | 1.075 | 0.158 | 1.266 | 1.047 |
| JARID1A | 0.264 | −0.044 | 0.193 | 0.024 | 0.248 | 0.14 | −0.789 | −0.641 | −0.51 | −0.182 | 0.036 | −1.029 | 0.104 |
| KARCA1 | −0.477 | 0.009 | −0.099 | −0.128 | −0.249 | −0.012 | −0.165 | 1.334 | 1.425 | 0.145 | 0.141 | −1.219 | −0.261 |
| KATNA1 | 2.1505 | 0.1875 | −0.0135 | 1.3535 | 1.1835 | −0.0045 | −0.6785 | −0.1075 | −0.0385 | −0.1735 | 0.0045 | −0.7955 | −0.1115 |
| KCNC2 | −0.02 | −0.159 | −0.325 | −0.108 | 0.051 | −0.089 | 0.114 | −0.501 | −0.389 | −0.912 | −0.102 | 1.255 | 0.639 |
| KCND2 | 0.243 | −0.318 | −0.874 | −0.211 | −0.64 | 0.121 | −0.908 | −0.442 | −0.472 | −0.831 | −1.073 | −0.302 | 0.543 |
| KCNK4 | 0.487 | −0.421 | 0.024 | 0.255 | 0.266 | 1.039 | −0.51 | 0.297 | 0.209 | 1.251 | −0.742 | 0.674 | −0.348 |
| KCNN4 | −0.0045 | 0.1595 | 0.2885 | −0.2455 | 0.0025 | 0.0295 | −0.9785 | −0.1265 | −0.1255 | −0.6685 | −0.9055 | 0.2105 | −0.0335 |
| KCNS3 | 0.2245 | −0.2695 | 0.6195 | −0.0535 | 0.8715 | 0.7785 | 0.1475 | 0.0875 | −0.0285 | 1.1455 | −1.2055 | 0.3745 | 0.5155 |
| KCTD15 | 0.552 | 0.083 | −0.058 | 0.385 | −0.552 | −0.207 | −0.134 | 0.08 | 0.034 | 0.614 | −0.63 | −1.247 | −1.333 |
| KCTD3 | −0.25 | −0.103 | 0.131 | 0.136 | −0.218 | 0.685 | 0.008 | −0.147 | −0.118 | −0.17 | −0.332 | −0.256 | −0.439 |
| KDELR2 | −0.049 | −2.006 | −0.118 | −1.172 | −1.521 | −0.25 | −2.493 | 0.108 | 0.216 | −0.808 | −0.12 | −0.285 | −0.165 |
| KIAA0020 | −1.009 | −0.183 | 0.03 | 0.564 | 0.648 | −0.171 | 0.232 | −0.422 | −8.36 | 0.787 | 2.513 | 0.048 | −0.355 |
| KIAA0101 | −0.2745 | −0.1345 | −0.8345 | −0.3695 | −0.5405 | −0.2455 | 0.0175 | −0.5975 | −0.5315 | −0.2695 | 0.8655 | −0.3835 | −0.1495 |
| KIAA0153 | −1.0495 | −0.2675 | −0.1805 | 0.0715 | 0.4415 | 0.4535 | 0.6325 | 0.3635 | 0.3655 | 0.1015 | −0.8675 | 0.7165 | 0.7595 |
| KIAA0182 | 0.1275 | −0.3485 | −0.0755 | −0.6735 | −0.3595 | −0.3095 | 0.3015 | −0.1385 | −0.1515 | −0.4485 | −0.1155 | −0.7265 | −0.2275 |
| KIAA0196 | 0.2945 | −0.3675 | −0.1415 | −0.3875 | −0.1725 | −0.3805 | −0.2435 | −0.1835 | −0.0655 | 0.7695 | 0.0305 | 0.5875 | 1.0455 |
| KIAA0241 | −0.695 | −0.316 | −0.999 | 0.038 | 0.501 | 0.175 | −0.734 | 0.265 | 0.107 | 1.273 | −0.349 | 1.195 | 0.549 |
| KIAA0251 | −0.3735 | −0.3495 | 0.3555 | 0.1995 | 0.2365 | 0.1855 | −0.3115 | −0.2485 | −0.2825 | −0.5055 | 0.0825 | 0.2355 | −0.0365 |
| KIAA0274 | 0.015 | 0.795 | 0.887 | −0.077 | −0.113 | −0.008 | 0.469 | −0.117 | −0.042 | −0.704 | 0.244 | 0.954 | 0.902 |
| KIAA0515 | −0.0545 | −0.0585 | −0.3405 | 0.1605 | −0.3925 | 0.2645 | −0.7045 | 0.1335 | 0.1685 | −0.0025 | 0.0555 | −0.2455 | 0.5615 |
| KIAA0582 | 0.4255 | 1.0555 | −0.4495 | −1.1525 | −0.9995 | −1.2815 | 1.5755 | −0.5985 | −0.5835 | 0.8455 | 0.7075 | 0.4865 | −0.2575 |
| KIAA0650 | 0.5445 | −0.0455 | −0.7145 | −0.5715 | −0.5735 | 0.1865 | −0.8425 | −0.2885 | −0.2915 | −0.0725 | −0.5295 | −0.3075 | −0.0205 |
| KIAA0703 | 0.038 | 0.501 | 0.484 | 0.156 | −0.211 | 8.207 | 0.767 | −0.556 | −0.489 | −0.255 | −0.501 | −0.406 | −0.531 |
| KIAA0830 | 0.1555 | −0.2755 | 0.0875 | −0.0355 | −0.0305 | −0.8385 | −0.5865 | 0.5545 | 0.4975 | 0.8115 | −0.2645 | 0.7275 | 1.0675 |
| KIAA0980 | −0.8685 | 0.7785 | −0.0105 | −0.0555 | 0.0225 | −0.1995 | −0.9185 | 0.4025 | 0.3655 | 0.0825 | −0.6385 | 0.6265 | 0.1055 |
| KIAA1324 | 0.056 | 1.122 | 0.489 | −0.132 | −0.312 | −0.037 | 0.416 | −1.098 | −1.041 | −0.538 | −0.525 | −0.174 | 1.825 |
| KIAA1363 | 0.548 | 1.38 | 1.156 | 0.945 | 1.903 | −0.035 | −0.430 | −0.115 | −0.045 | 1.316 | −1.131 | 1.411 | 0.871 |
| KIAA1683 | −0.7925 | −0.4695 | 0.2355 | −0.0835 | 0.1025 | 0.3785 | −0.7215 | −1.0675 | −1.3285 | −1.0665 | −0.1475 | 0.2545 | −0.2765 |
| KIF12B | 0.2845 | 0.1805 | 0.0165 | 0.0965 | −0.0095 | −0.0015 | 0.0145 | 0.2485 | 0.2875 | 0.2645 | −0.1375 | −0.5795 | −0.1915 |
| KIF24 | −0.4035 | 0.2725 | −0.5045 | −0.1445 | −0.1675 | −0.6005 | −0.0015 | −0.4065 | −0.4445 | −0.4435 | 0.0185 | 0.2175 | −0.0805 |
| KIN | 0.2055 | −0.9525 | 0.1135 | −0.1705 | −0.2145 | 1.3315 | −0.3805 | −0.6295 | −0.5105 | −0.8705 | −1.0885 | −0.2245 | −0.1975 |
| KIRREL3 | −0.0025 | −0.3505 | −0.5735 | −0.4525 | −0.0815 | 1.0205 | 0.1005 | 0.3925 | 0.4955 | −0.5405 | −0.5085 | 0.2515 | 0.3855 |
| KIT | −0.8735 | −0.8905 | 0.3355 | −0.8515 | 0.2225 | 1.5825 | 0.4535 | 6.3305 | 0.4705 | −1.9245 | −0.8465 | −1.9615 | 0.2805 |
| KLK5 | −0.004 | 0.004 | −0.668 | −0.74 | −0.059 | 1.69 | 0.236 | 0.083 | 0.273 | −0.204 | −0.383 | −0.037 | 0.751 |
| KLK6 | −1.249 | −0.189 | −0.26 | −0.722 | 0.408 | 0.72 | 0.523 | 0.073 | 0.128 | −1.386 | −0.45 | −1.535 | 0.744 |
| KLK7 | −1.009 | −1.488 | −0.761 | −1.284 | −1.075 | −0.362 | −2.593 | 0.035 | 0.022 | −0.486 | −8.442 | −1.084 | −0.345 |
| KLK8 | −0.2655 | −0.4775 | −0.4415 | −0.1095 | −0.7145 | −0.0115 | −0.5225 | −0.3005 | −0.1265 | −0.1065 | 0.1645 | −0.5185 | −0.8585 |
| KNTC2 | −1.22 | −0.029 | 0.366 | 0.123 | −0.781 | −0.112 | −0.417 | −1.528 | −1.306 | −0.169 | 0.25 | −0.933 | −1.417 |
| KPNA2 | 1.5305 | 0.7355 | 0.7975 | 0.5135 | 0.3875 | 0.2125 | 0.4805 | 1.0195 | 0.9955 | 0.1375 | 0.0105 | −0.0055 | 0.2055 |
| KREMEN2 | −1.0055 | −0.4045 | −1.0015 | −0.9575 | −0.1425 | −0.4135 | 1.7235 | −1.0215 | −1.0965 | −0.7565 | −1.2735 | −0.3965 | −0.7245 |
| KRT10 | −1.7625 | −1.0365 | −0.9075 | −1.4515 | −0.4115 | −0.2955 | 1.5325 | −2.0555 | −2.1635 | −1.4895 | −1.2235 | −0.7595 | −0.8415 |
| KRT13 | −0.822 | −0.303 | −1.054 | −1.35 | −0.007 | 0.597 | −0.3 | 0.589 | 0.599 | −1.328 | −1.12 | −0.141 | 1.542 |
| KRT15 | 0.852 | −0.212 | −0.268 | −0.02 | 0.603 | 0.117 | 1.26 | −0.224 | −0.102 | 0.802 | −0.331 | 0.765 | 0.5 |
| KRT17 | 0.8345 | −0.0795 | 0.2375 | −1.6175 | −0.0215 | 1.2345 | −0.2485 | −2.2095 | −2.2135 | −2.7575 | −2.3965 | 0.3525 | −1.9675 |
| KRT18 | −1.889 | −2.502 | −1.933 | −2.405 | 0.239 | 1.401 | 1.457 | 0.594 | 0.901 | −1.155 | −1.042 | 2.046 | 1.044 |
| KRT23 | −0.1095 | 0.2835 | −0.1755 | 0.0895 | −0.1795 | 0.2765 | 0.0475 | 0.2785 | 0.3165 | 0.0035 | −8.3245 | 0.1885 | 0.1695 |
| KRT6B | 0.628 | 0.055 | 0.828 | −0.22 | −0.306 | 0.609 | 2.866 | 0.918 | 0.56 | −0.219 | −0.16 | −0.14 | 2.068 |
| KRTAP3-3 | 2.8225 | 1.1695 | 2.0595 | −0.3665 | −1.0475 | 0.6275 | −0.0145 | 0.7555 | 0.5645 | −1.1635 | 1.2135 | 1.6685 | 2.2465 |
| KRTHA7 | 0.3445 | −0.8045 | −1.1655 | −0.2355 | −0.6175 | 1.2695 | −0.3555 | −0.2825 | −0.2905 | −0.2705 | −1.9615 | −0.6055 | 1.3645 |
| LAMA3 | −0.724 | −0.255 | 0.372 | 0.388 | 0.55 | 0.113 | 0.548 | −0.136 | −0.129 | 0.564 | 0.097 | 0.378 | 0.743 |
| LAMB3 | −0.351 | −0.107 | −0.065 | 0.181 | −0.413 | −0.18 | −0.28 | 1.234 | 0.895 | −0.45 | −0.235 | 1.6 | 0.508 |
| LASS2 | −0.5985 | −0.4515 | −0.2015 | 0.0415 | −0.1505 | −0.1185 | −1.2745 | −0.0025 | −0.0405 | −1.1115 | −0.8935 | 0.0995 | −0.9875 |
| LAX | −0.7975 | 0.7455 | −1.7175 | 1.0425 | −0.5875 | 0.5845 | −0.8595 | 0.2605 | 0.5435 | 0.2145 | 0.3925 | −0.2345 | 0.0285 |
| LBR | −0.9285 | −0.6285 | −0.9905 | −0.5705 | −0.8585 | −0.2585 | −1.5385 | 0.2335 | 0.4305 | 0.5445 | −1.2795 | −1.3005 | −0.9665 |
| LCE1B | −8.0675 | 0.3425 | 0.9765 | 0.3815 | −0.2325 | 0.9515 | −0.3535 | 0.7855 | 0.8715 | 0.2285 | 0.2585 | −1.2205 | 0.9925 |
| LDHB | 0.004 | −0.223 | −0.281 | 0.203 | −0.096 | −0.044 | −0.161 | 0.091 | 0.1 | 0.135 | −0.013 | 0.18 | −0.269 |
| LDOC1L | 0.787 | −1.373 | −0.007 | 0.11 | −0.55 | 0.523 | 1.176 | −0.511 | −0.449 | 0.307 | −0.587 | −0.036 | 1.354 |
| LETMD1 | −2.6235 | 0.1925 | −0.3195 | −0.1885 | −0.7985 | 0.7775 | −0.3875 | 0.8485 | 0.8365 | −0.2875 | −1.0085 | 1.0545 | 0.1785 |
| LFNG | −0.267 | −0.31 | 0.355 | 0.128 | 0.496 | 0.217 | 0.433 | −0.54 | −0.55 | 0.111 | 0.532 | −0.38 | 0.038 |
| LGALS2 | 0.327 | −0.195 | −0.133 | −0.948 | −0.66 | −0.283 | 0.198 | 0.28 | 0.456 | 0.459 | 0.439 | 1.555 | 1.161 |
| LGTN | 0.882 | −0.059 | −0.369 | 0.58 | 0.803 | 0.48 | 1.399 | 1.03 | 1.043 | 0.471 | −0.006 | 1.16 | 0.296 |
| LNX | 0.2215 | 0.6435 | 0.5575 | 0.3335 | 0.3555 | −0.4505 | −0.5375 | 0.4975 | 0.4915 | 0.3815 | 1.2285 | 0.4585 | 2.7905 |
| LOC11247 | −0.138 | −0.13 | 0.091 | 0.439 | −0.064 | 0.253 | −0.162 | −0.726 | −0.669 | −0.134 | −0.079 | −0.82 | 0.442 |
| LOC11492 | −0.149 | −0.063 | 0.559 | −0.13 | 0.207 | 0.233 | 0.343 | 0.35 | 0.368 | 0.401 | −0.179 | 0.448 | 1.292 |
| LOC11497 | 0.2975 | −0.0345 | −0.3005 | −0.2845 | −0.6255 | −0.3525 | −0.5326 | 0.0415 | 0.2715 | 0.0635 | −0.1695 | 0.2185 | −0.3945 |
| LOC11621 | 3.9115 | 2.7385 | 0.8445 | 1.2235 | 0.6105 | −0.0675 | −1.2745 | −2.5615 | −2.6755 | 1.2035 | −1.3915 | −0.6525 | −0.3655 |
| LOC11644 | 0.4635 | 0.0285 | 0.4035 | −0.1215 | 0.6925 | −0.4675 | −0.3795 | 0.9065 | 0.5195 | 0.7545 | 0.1545 | 1.1365 | 0.6945 |
| LOC12022 | 0.2655 | −0.7265 | 0.0405 | −0.2975 | 0.4985 | −0.1455 | 0.4035 | 0.0265 | −0.0015 | 0.1995 | −0.0795 | 1.2525 | 0.4395 |
| LOC12422 | 0.4665 | 0.0855 | 0.1105 | 0.0765 | −0.0205 | 0.2595 | 0.4645 | 0.3955 | 0.3365 | −0.8245 | 0.0365 | −0.2155 | 0.1395 |
| LOC14654 | 1.046 | 0.69 | 0.731 | −0.227 | −0.04 | −0.279 | 1.191 | 0.382 | 0.427 | 1.449 | 0.085 | 1.64 | 1.568 |
| LOC15506 | 1.241 | −0.372 | −0.237 | −0.188 | −0.118 | −0.006 | −0.062 | 2.132 | 2.254 | −0.431 | 1.389 | −0.64 | −0.911 |
| LOC20189 | 0.1085 | 0.1245 | −0.8165 | −0.3335 | −0.0375 | 0.5495 | −0.6075 | −2.0245 | −2.1875 | −0.3795 | 0.1865 | −1.6505 | −0.3875 |
| LOC25398 | 0.654 | 0.169 | −0.07 | −0.399 | 0.046 | 0.391 | −0.498 | −0.133 | −8.257 | −0.267 | −0.17 | −0.052 | −0.487 |
| LOC33990 | −0.296 | −0.254 | 0.018 | 0.115 | 0.45 | −0.353 | −0.348 | 1.054 | 0.951 | 0.344 | 0.42 | 0.709 | 0.914 |
| LOC34817 | −0.4375 | −0.7695 | 0.3765 | 0.4135 | −1.3235 | 0.0635 | −0.0565 | −0.0885 | −0.1485 | −0.6515 | 0.1555 | 0.4815 | −0.5375 |
| LOC40045 | 1.244 | 1.11 | −0.022 | 1.08 | 0.78 | 0.88 | −1.135 | −0.18 | −0.2 | 1.71 | 0.33 | −0.189 | −1.397 |
| LOC44089 | −0.445 | 0.021 | −0.483 | 0.122 | 0.187 | 0.249 | 1.177 | 0.606 | 0.493 | −0.151 | −0.207 | 0.582 | 0.408 |
| LOC49386 | 0.79 | −0.963 | −0.224 | 0.638 | 0.831 | 0.45 | 1.401 | −0.225 | −0.104 | 0.022 | −0.381 | 0.76 | 0.564 |
| LOC51123 | 0.845 | 0.381 | −0.051 | −0.133 | −0.068 | 0.019 | 0.002 | 1.264 | 1.174 | 0.695 | 0.611 | 0.721 | 0.202 |
| LOC51161 | −1.0855 | −0.9165 | 0.7315 | 0.1505 | 1.3205 | 0.5415 | −0.8795 | 0.7715 | 0.6025 | 1.2205 | −1.5035 | 0.9475 | 0.5025 |
| LOC51321 | 0.8215 | 0.1755 | 0.7705 | 0.1785 | −0.1555 | −0.2975 | −0.1475 | −0.1255 | −0.1305 | 0.1285 | −0.3355 | −1.2005 | 0.5155 |
| LOC51760 | 0.0516 | 0.5345 | 0.1585 | 0.2195 | −0.7085 | −0.3805 | 0.3495 | −1.3115 | −0.5525 | −0.5115 | 0.3415 | −0.6155 | −0.5395 |
| LOC81558 | 1.165 | −0.262 | 0.749 | 1.17 | 0.755 | 0.185 | 0.11 | 0.784 | 0.916 | 1.382 | 0.695 | 0.938 | 1.618 |
| LOC81569 | −0.0235 | 0.1735 | 0.1525 | −0.1865 | −0.3605 | −0.1275 | 0.5155 | 1.2045 | 1.1705 | 0.8085 | −0.0585 | 0.7305 | −9.2345 |
| LOC90355 | 0.403 | −0.87 | −0.268 | −0.772 | −0.791 | −0.413 | 1.317 | 0.572 | 0.629 | 0.051 | −0.448 | 0.059 | 0.408 |
| LOC90701 | 1.124 | 0.819 | −0.5 | 0.913 | 0.774 | −0.743 | −0.318 | −1.558 | −1.264 | −1.498 | 0.243 | 1.629 | −0.201 |
| LONP | 0.0445 | 0.3615 | 0.0885 | −0.1585 | −0.3215 | −0.0015 | 0.1485 | 0.6485 | 0.8735 | 0.7195 | 0.5285 | −0.0825 | 1.0755 |
| LRAP | −0.7965 | 2.1885 | 0.8585 | 0.7755 | 1.3525 | 0.6165 | −0.6035 | −0.1005 | −0.0765 | 0.1445 | −2.0725 | 0.6415 | 0.8525 |
| LRBA | −0.1085 | 0.5355 | 0.8715 | 0.1295 | 0.0535 | 0.0445 | −0.2745 | −0.5015 | −0.5545 | −0.1905 | −0.5825 | −0.7775 | −0.3035 |
| LRIG1 | −0.7355 | −0.5185 | −0.9935 | 0.2215 | −0.6965 | −0.4495 | −1.1995 | 0.3725 | 8.3795 | −0.8945 | 0.8835 | −1.7305 | −1.5985 |
| LRP6 | 2.645 | 1.734 | 1.771 | 2.684 | 2.525 | 1.591 | 0.761 | 2.273 | 2.244 | 1.76 | 1.211 | −0.877 | −1.035 |
| LRP8 | 0.758 | −0.147 | 0.62 | 0.871 | 1.051 | 0.818 | 0.473 | 0.176 | 8.22 | 1.031 | 0.585 | 0.339 | 1.35 |
| LRRC17 | 2.7765 | 0.3416 | −0.1995 | −0.1905 | 0.4785 | −0.6285 | −0.4785 | 0.7775 | 0.8175 | 1.1965 | 2.4245 | −0.6185 | 2.2165 |
| LRRC2 | 0.9416 | 0.1795 | 0.1135 | 0.0735 | −0.0295 | −0.2315 | 0.1485 | 0.4945 | 0.3805 | 0.3935 | 0.0385 | 0.6345 | −0.3865 |
| LSM1 | −1.4885 | −0.5975 | −1.9995 | 0.1915 | −0.6525 | 0.6535 | −1.0975 | 2.2865 | 2.2615 | 8.1685 | −1.2485 | 3.1695 | 0.9525 |
| LSM8 | −0.116 | 0.05 | −0.265 | −0.059 | −0.066 | 0.015 | −0.121 | −0.258 | −0.268 | −0.112 | −0.268 | 0.112 | 0.124 |
| LTB | −0.012 | −0.754 | −0.552 | 0.369 | 0.065 | 0.358 | −0.41 | −0.291 | −0.13 | −0.355 | −1.092 | 0.417 | −0.929 |
| LY6D | −0.775 | −1.012 | −0.755 | −1.401 | −1.267 | −0.607 | −1.855 | 0.458 | 0.432 | −0.201 | 0.329 | −0.005 | 0.435 |
| LYN | 0.742 | 0.091 | 0.435 | 0.04 | 0.094 | 0.004 | 0.602 | 0.549 | 0.5 | 0.294 | 0.137 | −0.033 | 0.183 |
| MA02L1 | −0.397 | 0.224 | 0.926 | 0.086 | 0.345 | 0.055 | −0.143 | 1.076 | 1.681 | −0.27 | −0.181 | −0.261 | −0.335 |
| MADP-1 | −0.6425 | −2.2205 | 0.3555 | 0.0085 | 0.0365 | −0.1925 | −0.0545 | −1.0825 | −2.1315 | −1.3335 | −0.0615 | −0.0225 | −1.1135 |
| MAGEA12 | −0.4 | 0.58 | −0.694 | 0.49 | 0.51 | 0.25 | 0.132 | −0.44 | −0.04 | −0.32 | −0.08 | 0.334 | −0.505 |
| MAGEA2 | −0.5305 | 0.3295 | 0.7765 | −0.0205 | 0.0075 | −0.4285 | 0.0525 | 2.1615 | 1.9515 | −1.3115 | 0.3565 | −0.4035 | −0.7155 |
| MAGEA3 | 1.6905 | 0.5225 | −0.0375 | 0.0845 | 0.2895 | 0.3975 | −0.5925 | 1.9185 | 1.8575 | 0.5745 | 0.8155 | −0.8075 | −1.4885 |
| MAGEA4 | −1.157 | −0.63 | −0.043 | −0.432 | −0.67 | 0.344 | −1.427 | 0.054 | 0.028 | −0.189 | −1.407 | 1.143 | 0.164 |
| MAGED4 | 2.1795 | 0.3635 | 0.3295 | 1.0125 | 0.8075 | 1.4635 | −0.3245 | 0.6555 | 0.5735 | 0.5385 | 0.1895 | 0.2275 | −0.0195 |
| MAGI1 | 1.8145 | 1.1005 | −0.2375 | −0.7265 | 0.7555 | 1.2465 | −1.0595 | 0.4205 | 0.3025 | 0.2795 | −0.5975 | 0.4125 | 0.7015 |
| MAGI-3 | 0.821 | 0.045 | −0.2 | −0.227 | −0.004 | −0.304 | −0.302 | 0.054 | 0.023 | −0.336 | −0.25 | 0.644 | −0.515 |
| MAL2 | 0.353 | −0.076 | −0.023 | −0.078 | 0.143 | 0.905 | 1.486 | 2.365 | 1.747 | 0.891 | 0.632 | 1.262 | 1.307 |
| MAN2B1 | −0.3755 | 0.3995 | −1.1195 | −0.6115 | −0.5315 | −0.7135 | −0.7575 | 1.0885 | 1.1945 | 0.3345 | −1.0495 | 0.2465 | −0.3595 |
| MAPK8IP2 | −1.3315 | 0.4705 | −1.0415 | 0.1535 | −0.4475 | −0.0815 | 0.0055 | 0.3615 | 0.3845 | 0.0725 | −0.2665 | 0.2225 | −0.1405 |
| MARCO | 0.402 | 0.667 | −1.405 | −0.411 | 2.013 | 0.905 | 0.711 | −0.903 | −0.828 | 0.472 | −0.301 | 0.434 | −1.297 |
| MARLIN1 | −0.041 | −1.944 | −0.324 | −0.205 | −0.158 | −0.105 | −0.487 | −0.178 | −0.173 | −0.772 | −0.218 | −0.122 | 0.075 |
| MASS1 | −0.1265 | −0.6135 | −0.3065 | −0.3955 | 0.1825 | −0.4145 | −0.4515 | −0.5115 | −0.3795 | −0.6545 | −0.5015 | −0.9535 | −0.2635 |
| MCM2 | 0.9155 | 0.7895 | −0.3275 | 0.5505 | −0.7285 | −0.1005 | −0.4355 | −0.1705 | −0.1525 | −0.3285 | 1.0175 | −0.4145 | −0.2025 |
| MCM3 | 0.724 | −1.362 | −0.367 | 0.358 | 0.176 | −0.378 | 0.424 | −1.66 | −1.487 | 0.183 | −0.727 | 0.533 | 0.41 |
| MESP1 | 1.4705 | −0.3065 | 0.4225 | 2.1065 | 1.5215 | 0.8585 | −0.0715 | −0.2495 | −0.1375 | 0.6275 | 0.5545 | −0.6805 | −2.1135 |
| METRN | −0.0505 | −0.1425 | 0.2885 | −0.1005 | −0.0735 | −0.0025 | −0.7235 | −0.1675 | −0.3815 | −0.9635 | 0.1745 | −0.5345 | −0.8835 |
| MFAP2 | −0.188 | −0.187 | −0.183 | 0.16 | 0.571 | −0.07 | 0.165 | −0.225 | −0.169 | −0.348 | 0.578 | −0.394 | 0.37 |
| MFGE8 | −0.0275 | 0.0475 | −0.5085 | 0.2105 | −0.1105 | −0.1295 | −0.1805 | −0.2225 | −0.1605 | −0.0515 | −0.4745 | −0.3815 | 0.3575 |
| MGAT4B | −0.737 | −0.125 | −0.504 | −0.106 | −0.287 | −0.033 | 0.071 | 0.405 | 0.338 | −0.338 | −0.37 | 0.799 | −0.24 |
| MGC1091 [symbol] | 2.0045 | 1.3495 | −0.0275 | −0.4705 | 0.0065 | 0.1305 | 1.2945 | 0.3675 | 0.2205 | 0.7875 | −0.1085 | 1.0945 | −0.1335 |
| MGC1098 [symbol] | −0.1665 | 0.1055 | −0.5575 | −0.3395 | −0.9245 | −0.2395 | −0.7035 | −0.6505 | −0.5045 | −0.4545 | −0.5465 | −1.8015 | 0.1205 |
| MGC1124 [symbol] | 0.492 | 0.474 | −0.219 | 0.068 | 0.156 | −0.177 | −0.807 | −0.414 | −0.298 | −0.288 | −0.494 | −1.143 | −0.358 |
| MGC1127 [symbol] | −0.341 | −0.146 | 0.119 | −0.195 | −0.023 | −0.144 | 0.114 | −0.285 | −0.174 | −0.835 | 0.46 | −0.798 | −1.203 |
| MGC1312 [symbol] | −0.495 | 0.307 | 0.037 | −0.412 | −0.231 | −0.727 | 0.111 | 0.517 | 0.455 | 0.513 | −0.986 | 0.205 | 0.287 |
| MGC1318 [symbol] | −0.027 | 0.348 | 0.085 | 0.328 | 0.017 | 0.091 | 0.425 | 0.403 | 0.37 | 0.284 | −0.555 | 0.289 | −0.081 |
| MGC1414 [symbol] | 0.5615 | 0.1495 | 0.3025 | 0.1655 | 0.1375 | 0.3755 | 0.0825 | 0.1885 | 0.1355 | 0.2595 | −0.0285 | −0.2675 | −0.4365 |
| MGG1428 [symbol] | 0.673 | 0.129 | −0.138 | 0.039 | −0.618 | −0.129 | −0.483 | 0.673 | 0.85 | 0.716 | −0.402 | 4.014 | −0.582 |
| MGC1481 [symbol] | 0.489 | −0.657 | −0.593 | −0.403 | −0.688 | −0.448 | 2.622 | 0.497 | 0.775 | 1.409 | −0.394 | −0.601 | 0.783 |
| MGC1580 [symbol] | −0.1105 | −0.1985 | 0.2045 | 0.1005 | −0.2035 | 0.1595 | −0.3725 | −0.0765 | −0.0535 | 0.3905 | 0.0035 | 0.1805 | 0.3465 |
| MGC1663 [symbol] | −0.321 | −0.352 | −0.109 | −0.509 | −0.49 | −0.116 | −0.837 | 0.038 | −0.029 | −0.23 | −0.148 | −0.29 | −0.113 |
| MGC1873 [symbol] | 1.22 | −0.453 | −0.261 | 0.852 | 1.528 | 0.191 | −0.089 | −0.324 | −0.274 | 0.129 | −0.075 | −0.611 | −0.303 |
| MGC2053 [symbol] | 1.207 | −0.148 | 1.04 | 0.329 | 0.898 | 0.159 | 0.075 | 0.512 | 0.475 | 1.764 | 0.959 | 1.254 | 1.186 |
| MGC2398 [symbol] | 0.256 | 0.451 | −0.09 | 0.197 | −0.171 | −0.249 | −0.089 | −0.405 | −0.351 | 0.554 | −0.312 | −0.646 | −0.726 |
| MGC2610 | −1.477 | −0.519 | −0.409 | −0.136 | −0.739 | −0.865 | −0.549 | 0.428 | 0.93 | −0.39 | −0.892 | 2.391 | 1.912 |
| MGC2714 | −0.978 | −0.968 | −0.561 | −0.06 | −0.645 | −0.434 | −0.878 | 1.416 | 2.061 | −0.199 | −0.892 | 2.305 | 2.338 |
| MGC2716 [symbol] | −1.2175 | −1.4225 | −0.4645 | 0.0145 | −0.5755 | −0.4585 | −0.8025 | 1.4915 | 1.8955 | −0.1495 | −0.8125 | 1.7565 | 2.2785 |
| MGC2716 [symbol] | −1.3315 | −1.0485 | −0.8335 | −0.1975 | −1.0295 | −0.7605 | −0.9245 | 1.7395 | 1.9425 | 0.3765 | −0.7515 | 2.7535 | 2.1205 |
| MGC2716 [symbol] | −1.31 | −1.335 | −0.654 | 0.131 | −0.909 | −0.584 | −0.942 | 1.333 | 1.019 | −0.323 | −0.87 | 2.132 | 1.691 |
| MGC2716 [symbol] | 0.0585 | −0.2495 | 0.2585 | −0.0275 | 0.2485 | 0.4825 | 0.4325 | −0.1225 | −0.1325 | 0.0155 | −0.7755 | −0.0375 | −0.1025 |
| MGC2716 [symbol] | 0.393 | 0.655 | 0.506 | 1.034 | 1.235 | −0.03 | 1.59 | 0.178 | 0.186 | 0.953 | −0.03 | −0.525 | 1.193 |
| MGC2887 [symbol] | −0.583 | 0.375 | −0.567 | −0.252 | −0.277 | 0.341 | −0.396 | 0.416 | 0.389 | −0.042 | −0.059 | 0.275 | −1.089 |
| MGC3212 [symbol] | −1.481 | −0.176 | −0.446 | 0.184 | −0.415 | 0.14 | −0.295 | −0.083 | −0.11 | −0.778 | −1.053 | 0.161 | 0.32 |
| MGC3321 [symbol] | −0.3475 | −0.4095 | −0.6485 | −0.4065 | 0.3315 | −0.4335 | 0.1845 | −0.1016 | −0.1475 | −0.7435 | −0.9145 | −0.2505 | −1.2365 |
| MGC3484 [symbol] | 0.2425 | 0.6535 | 0.1655 | −0.3945 | 0.2155 | 0.7735 | −0.0685 | −0.5295 | −0.6345 | −0.0655 | 0.4385 | 1.3615 | 0.0655 |
| MGC3492 [symbol] | 2.376 | 1.344 | 1.203 | 0.1 | 0.273 | 0.678 | 2.078 | 2.113 | 2.173 | −0.364 | 0.244 | 0.518 | 1.575 |
| MGC4021 [symbol] | −0.024 | 0.518 | 0.536 | −0.335 | 0.052 | 0.347 | −0.039 | 0.661 | 0.59 | 0.326 | −0.372 | −0.229 | −0.058 |
| MGC4251 | 0.0035 | −0.2305 | 0.0495 | −0.1476 | 0.0025 | −0.1225 | 0.4155 | 0.3595 | 0.2815 | 0.0455 | −0.8425 | 0.6065 | 0.6185 |
| MGC4308 | 1.05 | −0.227 | −0.419 | 0.905 | 1.111 | 0.372 | 1.475 | 1.279 | 1.249 | 1.484 | −0.099 | 1.005 | 0.8 |
| MGC4800 | −1.8835 | −1.0055 | −0.5875 | −1.3155 | −0.2655 | 1.6545 | −0.5665 | −0.2355 | −0.1855 | −1.7455 | −0.8955 | 1.9165 | 0.8865 |
| MGC4659 | −0.743 | −0.829 | 0.266 | −0.112 | −0.38 | 0.003 | −0.675 | −0.67 | −0.672 | −0.901 | −0.778 | −0.809 | −0.801 |
| MIA | −1.2525 | −0.4735 | 0.0745 | −0.4215 | −0.8985 | 0.0705 | −0.3795 | −0.4795 | −0.3955 | −0.7565 | −0.4505 | −1.2795 | −0.8485 |
| MID1 | −1.0095 | −0.4835 | 0.8936 | −0.3455 | −0.7355 | 0.0795 | −0.9715 | −0.4875 | −0.5295 | −0.9255 | −1.2085 | −1.6065 | −1.1055 |
| MID1 | −0.516 | −1.593 | −0.282 | −1.156 | −1.156 | −0.223 | −1.383 | 0.074 | 0.004 | −0.22 | 0.28 | −0.547 | −0.325 |
| MID1 | −0.6175 | 0.3565 | −0.8205 | −0.1305 | −0.4025 | −0.0525 | −0.7505 | −0.1535 | −0.3455 | 0.5515 | −0.7165 | 1.9985 | −0.8785 |
| MLF1IP | 2.2935 | 0.8235 | −1.8805 | 0.1995 | −0.4175 | 0.8235 | −0.8035 | −0.1825 | −0.0075 | 0.5725 | 0.1475 | 0.2475 | −0.2865 |
| MLSTD1 | −1.037 | −1.181 | −1.341 | 0.351 | −0.582 | −0.593 | 1.874 | 0.247 | 0.373 | −0.628 | −0.946 | 2.563 | −0.278 |
| MMP1 | 0.381 | 0.192 | 0.421 | −0.435 | −0.354 | 0.035 | 0.459 | 0.098 | −0.016 | 0.724 | −0.128 | 0.278 | 0.089 |
| MMP7 | 0.1215 | −0.7505 | −0.1495 | −0.1095 | 0.1995 | −0.2425 | 0.3435 | 0.1535 | 0.1085 | −0.1855 | −0.3885 | 0.6535 | 0.3165 |
| MNAT1 | −0.6245 | −0.2715 | −0.6985 | −0.8955 | −0.5815 | 0.1265 | −0.0855 | 0.1435 | 0.1585 | −0.1195 | 0.0205 | 0.3825 | −1.4145 |
| MRPL14 | 0.089 | −0.038 | −0.655 | −0.055 | −0.152 | 0.001 | 0.204 | 0.779 | 0.921 | 1.064 | 0.107 | −1.031 | −0.233 |
| MRPL15 | −0.0685 | 0.0505 | −0.1565 | 0.4545 | 0.0915 | 0.0265 | −0.0345 | 0.5025 | 0.5005 | 0.3545 | 0.1355 | 0.2755 | 0.2335 |
| MRPL27 | 0.4705 | 0.9255 | 0.6635 | 0.0245 | 0.0085 | −0.6785 | 1.0955 | 0.9115 | 0.8055 | 0.9495 | −0.3815 | −0.6075 | 0.1135 |
| MRPL34 | 1.034 | −0.158 | 0.548 | −0.469 | −0.009 | −0.057 | −0.138 | 0.62 | 0.578 | 0.216 | −0.062 | 0.198 | 0.342 |
| MRPL43 | 0.1995 | 0.6055 | −0.3805 | 0.0275 | −0.1605 | −0.0365 | 0.2505 | −0.2675 | −0.1485 | 0.2395 | −0.4445 | 0.2345 | −0.5035 |
| MRPL45 | −0.66 | −0.871 | 0.201 | −0.204 | 0.402 | 0.025 | −0.049 | −0.587 | −0.499 | −0.012 | −0.002 | −0.747 | 1.185 |
| MRPL48 | 0.0145 | −0.1875 | 0.4125 | 0.0395 | 0.2175 | 0.3235 | 0.7735 | 0.0145 | 0.1075 | 0.5575 | 0.2905 | 0.8365 | −0.0305 |
| MRPL9 | −0.4145 | −0.3855 | −0.0675 | −0.2155 | −0.1355 | −0.5075 | 0.0085 | −0.0545 | 0.0255 | −0.1295 | 0.0215 | −0.1475 | −0.0965 |
| MRPS14 | 0.467 | 0.065 | 0.133 | −0.258 | −0.331 | −0.298 | −0.102 | −0.516 | −0.502 | 0.102 | −0.399 | −0.62 | −0.22 |
| MRPS15 | 0.137 | −0.353 | −0.082 | −0.299 | −0.24 | −0.204 | 0.258 | 0.307 | 0.257 | 0.242 | −0.103 | −0.586 | −0.292 |
| MRPS17 | 0.202 | −0.568 | −0.283 | −0.444 | −0.427 | −0.122 | −0.138 | 0.327 | 0.316 | −0.147 | 0.263 | −0.618 | −0.543 |
| MRPS24 | 0.5375 | 0.3235 | 0.2975 | 0.1435 | 0.4245 | 0.0945 | 0.0485 | −0.2345 | −0.2115 | 0.0635 | 0.2245 | −0.0515 | 0.3695 |
| MRPS25 | 0.138 | 0.216 | 0.573 | 0.878 | 0.022 | −0.088 | −0.296 | −0.003 | −0.047 | 0.308 | 0.049 | −0.214 | 0.517 |
| MRPS31 | −0.595 | −0.12 | 0.002 | 0.378 | −0.73 | −0.045 | 0.007 | 1.672 | 1.676 | 0.242 | −0.21 | 0.659 | 0.874 |
| MRPS6 | −0.0995 | 0.1285 | 0.1495 | −0.2595 | −0.4215 | −0.1675 | −0.2605 | 0.0755 | 0.1285 | 0.1295 | 0.7375 | −0.5985 | −0.0105 |
| MS4A1 | 1.4185 | 0.9875 | 0.6175 | −0.0865 | 0.8815 | 0.5625 | 1.0345 | 0.2855 | 0.3035 | 0.5235 | −0.1545 | −0.6325 | −0.7325 |
| MSH2 | 3.198 | 0.447 | 0.693 | 0.324 | −0.833 | 0.348 | −0.28 | 4.994 | 4.503 | 1.587 | 0.704 | 3.811 | 0.211 |
| MSH3 | −0.608 | −0.069 | 0.165 | 0.857 | 0.247 | −0.112 | 0.196 | −0.31 | −0.288 | 0.251 | −0.851 | −0.008 | −0.578 |
| MSMB | 0.336 | 0.397 | 0.206 | −0.359 | −0.123 | −0.014 | −0.474 | −0.156 | −0.331 | 0.194 | 0.367 | 0.129 | −0.316 |
| MTA1 | −0.1305 | 0.3065 | 0.1705 | −0.2855 | 0.4675 | −0.1195 | 0.0325 | −0.6325 | −0.5295 | 1.2425 | −1.2265 | 0.8935 | 0.0375 |
| MTA3 | −0.035 | 0.303 | −0.451 | 0.024 | 0.121 | −0.822 | 0.663 | 0.099 | 0.052 | −0.165 | −0.164 | 0.108 | −0.092 |
| MTAC2D1 | −0.528 | −0.216 | 0.309 | 0.296 | −0.158 | −0.194 | −0.436 | −0.819 | −0.617 | −0.309 | −0.631 | 0.369 | −0.572 |
| MTCH1 | 1.0425 | 0.1485 | −0.2215 | 0.3495 | −0.0295 | 0.0115 | −1.0345 | −0.4645 | −0.3235 | −0.1235 | −0.1705 | −0.9285 | −0.9075 |
| MTHFD1 | 0.6775 | 1.0165 | −0.6845 | 1.2135 | 1.7825 | 0.5075 | 1.4485 | −0.5945 | −0.6115 | 0.9485 | −3.0615 | 1.2605 | 1.1715 |
| MTMR2 | 0.4265 | −0.4145 | −0.1475 | −0.2615 | −0.1595 | −0.4645 | 2.9495 | 0.8175 | 0.9845 | 0.1785 | −0.5865 | −0.4205 | −0.4815 |
| MUC1 | −0.157 | −0.831 | −0.273 | 0.883 | 0.682 | 0.708 | 0.267 | −1.093 | −1.085 | 0.168 | 1.081 | 0.633 | 0.005 |
| MUC20 | −0.715 | 0.856 | 0.216 | 0.019 | 0.014 | 0.292 | −0.243 | 0.567 | 9.995 | −0.286 | −0.565 | −1 | −0.036 |
| MUM1 | −0.305 | 1.957 | 2.004 | 0.886 | 1.503 | 1.55 | 1.105 | 0.776 | 0.759 | 0.396 | 1.323 | 0.994 | 2.33 |
| MXI1 | −0.777 | 0.728 | 1.683 | −0.392 | −0.822 | 1.058 | −1.627 | 0.278 | 0.298 | 0.538 | 0.458 | 1.11 | 0.068 |
| MYB | −1.339 | −2.475 | −0.855 | −1.551 | −1.289 | −0.651 | −2.306 | −1.143 | −1.095 | −1.416 | −0.362 | −0.485 | −0.943 |
| MYBL1 | 0.139 | 0.096 | −0.927 | −0.301 | −0.181 | −0.356 | −1.498 | −0.602 | −0.565 | −1.145 | −0.494 | 0.273 | −1.304 |
| MYBL2 | 0.128 | −0.095 | 0.36 | 0.213 | 1.038 | −0.054 | −0.01 | −0.402 | −0.385 | 0.438 | −0.065 | 0.277 | 1.227 |
| MYO10 | 0.0855 | −0.3015 | −0.1965 | −0.0735 | 0.5015 | −0.1585 | −0.6215 | 0.2015 | 0.3115 | −0.2385 | 0.2075 | 0.0145 | 2.0555 |
| MYO6 | 0.7535 | 0.4715 | 0.6375 | 0.6325 | −1.4575 | 0.4615 | −1.4335 | 0.7545 | 0.7995 | −1.4225 | −0.6215 | −1.6015 | −2.1725 |
| MYST3 | 0.0605 | −0.1225 | −0.1065 | 0.1135 | 0.2095 | 0.2575 | 0.1915 | 1.1625 | 0.6435 | 0.2985 | −0.0595 | 0.5335 | 0.1555 |
| NALP2 | −0.2455 | 0.1215 | −0.0655 | −0.2435 | −0.1935 | 0.0015 | 0.2985 | −0.2755 | −0.1905 | 0.4555 | −0.3955 | 0.4365 | −0.2815 |
| NALP6 | 0.2425 | −0.0615 | −1.1675 | −0.2275 | −0.3515 | −0.2415 | −0.0125 | 0.5095 | 0.4975 | −0.5325 | 0.7355 | 0.1555 | −0.7225 |
| NANS | 0.246 | −0.313 | −0.507 | 2.028 | 2.631 | 0.836 | −0.228 | 2.954 | 2.644 | 3.726 | −0.39 | 0.436 | 1.912 |
| NARF | −1.239 | −1.137 | 0.835 | 0.002 | 0.491 | 0.655 | −0.002 | −1.236 | −1.14 | −1.49 | −0.367 | −1.439 | 0.822 |
| NAT2 | 0.1695 | −0.4335 | −0.6125 | −0.0145 | 0.0605 | −0.0325 | 0.0285 | −0.4985 | −0.5555 | −0.0485 | −0.4815 | 0.8635 | 0.6335 |
| NAV2 | 0.289 | 0.782 | 0.038 | −0.19 | −0.164 | −0.143 | 0.346 | 0.981 | 0.974 | 0.535 | 0.427 | 0.359 | 0.139 |
| NDE1 | −0.8045 | −0.4655 | −0.1275 | −0.5345 | −0.3255 | 0.0465 | −0.2885 | 0.0445 | −0.0505 | −0.4635 | −0.4745 | 0.5945 | −0.6345 |
| NDP52 | 0.210 | −0.081 | 0.006 | −0.115 | −0.018 | 0.055 | 1.059 | 0.933 | 0.909 | 0.044 | 0.697 | 0.903 | 0.624 |
| NDRG2 | 0.003 | 0.202 | 0.394 | 0.19 | −0.098 | 0.204 | 0.33 | −0.632 | −0.655 | 0.612 | −0.718 | 0.908 | 0.506 |
| N0UFA7 | 0.2975 | −0.3415 | −0.1765 | −0.2945 | 0.4145 | −0.3055 | 0.5045 | −0.0735 | −0.0825 | 0.3745 | 0.0085 | 9.4915 | 0.3675 |
| NDUFB1 | 0.388 | 0.098 | −0.086 | 0.192 | −0.134 | −0.266 | 0.001 | 1.614 | 1.401 | 0.398 | 0.167 | −0.263 | −0.529 |
| NDUFB10 | 0.141 | 0.387 | 0.205 | −0.057 | −0.314 | −0.266 | 0.103 | 1.044 | 0.884 | 0.876 | −0.184 | 0.084 | −0.21 |
| NDUFB2 | 0.3445 | 0.4115 | 0.6425 | 0.1915 | −0.0575 | 0.2625 | 0.1805 | 0.4395 | 0.4135 | 0.3345 | −0.1325 | 0.0705 | 0.0895 |
| NDUFB5 | 0.7245 | 0.9855 | 1.0685 | 0.0055 | 0.1935 | −0.1605 | 0.1635 | 0.0945 | 0.1925 | 0.9565 | −0.0505 | 0.3245 | −0.2035 |
| NDUFB6 | 0.8005 | −0.3255 | 0.0555 | −0.4885 | −0.4615 | −0.1315 | 0.1155 | 0.7065 | 0.4555 | 0.3495 | −0.4305 | 1.0975 | 0.4385 |
| NDUFC2 | −1.8025 | −0.9435 | −1.4235 | −1.3985 | −0.6075 | 0.1535 | −2.6325 | −0.1455 | −0.0285 | −0.8275 | −1.2885 | 0.0415 | −0.3335 |
| NEBL | −0.139 | −0.006 | −0.444 | 0.543 | 0.035 | 0.266 | −0.253 | −0.458 | −0.364 | −0.001 | −0.198 | 0.036 | −0.689 |
| NEK2 | 1.284 | −1.209 | −0.727 | −1.178 | −0.854 | 0.788 | −0.977 | 1.828 | 1.727 | 1.861 | −1.022 | 0.441 | 0.562 |
| NEK6 | −0.6005 | −0.2775 | −1.1365 | 0.1825 | −0.6185 | 0.0285 | −0.2325 | 1.4575 | 1.2185 | 0.0955 | −0.2285 | −0.8315 | −0.3345 |
| NELL2 | −0.0945 | −0.3655 | −0.1515 | 0.3745 | 0.1445 | 0.1845 | −0.9635 | −0.1155 | −0.1155 | 0.4245 | 0.0245 | −0.5225 | −0.0705 |
| NEURL | −0.507 | 0.891 | 1.524 | 0.009 | 0.534 | 0.758 | −0.906 | −1.32 | −1.393 | 0.45 | −1.028 | 1.477 | −0.977 |
| NFE2L3 | −0.8785 | −0.1065 | −0.8755 | −1.1655 | 0.2245 | −0.5225 | −1.3635 | −0.5215 | −0.4925 | −1.3285 | −1.2685 | −0.7695 | −0.4135 |
| NFIA | 0.206 | 0.655 | 0.548 | 0.269 | 0.37 | 0.202 | −0.126 | 0.241 | 0.215 | 0.847 | −0.581 | 1.337 | 0.481 |
| NFIB | −0.1435 | 0.3105 | 0.0855 | 0.3635 | −0.1885 | −0.0035 | −0.3205 | 0.2385 | 0.3125 | 0.9355 | 0.3535 | −0.4195 | −0.0785 |
| NINJ1 | 0.2065 | 0.0545 | −0.1365 | 0.0475 | 0.2215 | −0.3125 | 0.0825 | −0.1305 | 0.0105 | −0.0845 | −0.0245 | 0.2055 | −0.0565 |
| NIPSNAP [symbol] | −0.1585 | −0.1245 | −0.5185 | −0.1625 | −0.3075 | 0.1745 | −0.3325 | 1.1415 | 1.1225 | −0.1635 | 0.1145 | −0.3665 | −0.7645 |
| NKX8-2 | −0.1535 | 0.1825 | −0.4155 | −0.1455 | −0.3815 | 0.0765 | 0.0505 | 1.2975 | 1.4785 | 0.2585 | 0.1295 | −0.7615 | −0.9965 |
| NME1 | 0.02 | 0.239 | 0.322 | −0.11 | 0.689 | −0.492 | 0.924 | 0.85 | 0.779 | 0.653 | 0.222 | 0.312 | 0.433 |
| NME2 | −1.1905 | 0.0765 | 0.1145 | 2.7595 | 0.1205 | 0.0765 | 3.7395 | 0.9525 | 1.1995 | 2.4215 | −2.0715 | 2.5445 | 2.2665 |
| NME3 | 0.0675 | −0.0095 | 0.5545 | −0.0295 | −0.1895 | 0.1205 | −1.4705 | 0.3105 | 0.2605 | 0.8205 | 0.7005 | −0.5475 | −0.7595 |
| NMES1 | 0.529 | −0.389 | −0.055 | −0.053 | −0.445 | −0.291 | 0.64 | −0.141 | −0.162 | 0.096 | 0.527 | −0.554 | −0.182 |
| NMI | 0.0105 | −0.1705 | 0.3735 | −0.0645 | −0.1215 | 0.2645 | −0.0315 | −0.0455 | −0.0235 | −0.1675 | −0.1035 | −1.1095 | −0.3725 |
| NOC4 | −0.5105 | −0.2805 | −0.5295 | −0.3935 | −0.4615 | −0.4145 | −0.2855 | 1.1705 | 1.3675 | −0.2295 | −0.4365 | 1.5495 | 2.0776 |
| N0LC1 | −0.6225 | 0.5165 | 0.3705 | 0.2215 | −0.6495 | −0.4705 | −1.2945 | −0.4935 | −0.3855 | −0.0045 | −0.8985 | −0.4935 | −0.3135 |
| NotFound | 0.2605 | 0.5205 | 0.2085 | 0.2215 | 0.3325 | 0.0025 | 0.4435 | 9.1205 | 0.1575 | 0.3625 | 0.2775 | −0.0985 | 0.1255 |
| NotFound | −0.896 | −0.519 | −0.181 | −0.428 | −0.644 | −0.517 | −0.686 | 0.186 | 0.261 | −0.663 | −0.582 | 1.097 | 0.801 |
| NotFound | 0.121 | 0.181 | −0.452 | 0.050 | −0.289 | −0.782 | 1.854 | 1.492 | 1.208 | 0.754 | 0.402 | 1.371 | 0.229 |
| NotFound | −0.69 | −0.042 | −0.186 | 0.259 | −0.685 | 0.11 | −0.306 | 1.476 | 1.818 | 0.408 | −0.688 | 0.645 | 0.549 |
| NotFound | −0.9585 | −0.7085 | −0.1175 | 0.5735 | 0.5485 | −1.5415 | −0.2445 | 0.9345 | 0.9295 | 0.3905 | −0.8525 | 0.1815 | −0.4595 |
| NotFound | −0.853 | −0.003 | 0.003 | 0.827 | −0.175 | 0.707 | −0.391 | 0.331 | 1.013 | −1.168 | −0.581 | 0.047 | 1.853 |
| NotFound | 0.3475 | −1.6905 | 0.3335 | −1.5125 | −0.9545 | −0.9545 | 2.7545 | 2.3675 | 2.4215 | 0.9835 | −1.0105 | −0.9265 | −0.9955 |
| NotFound | −0.2 | −0.489 | −0.941 | 0.447 | 0.048 | −0.542 | 0.309 | 0.232 | 0.209 | 0.194 | −0.157 | 0.34 | −0.925 |
| NotFound | 0.5395 | −0.0505 | −0.2475 | 0.8025 | 0.6045 | 0.0105 | −0.7445 | −0.5845 | −0.6395 | −0.5695 | −0.3435 | −0.1325 | −0.8455 |
| NotFound | −2.8475 | −0.6505 | −0.8675 | −0.4145 | −1.5845 | −0.7305 | −1.5985 | 1.5945 | 0.6895 | −0.3655 | −1.1885 | 1.8525 | 1.2635 |
| NotFound | −0.5745 | −0.3295 | −0.4335 | −0.2345 | 0.3505 | −0.1985 | −0.3015 | −0.2385 | −0.1975 | −0.2685 | −0.5895 | 0.5585 | 0.4175 |
| NotFound | −0.2135 | −0.0565 | −0.1135 | 0.0865 | −0.0835 | −0.0205 | 0.0665 | −0.3305 | −0.3385 | −0.3195 | −0.0925 | 0.6905 | −0.1395 |
| NotFound | 0.48 | 1.203 | 2.067 | −0.646 | −0.168 | 0.931 | −0.458 | 1.265 | 1.06 | 0.503 | 2.348 | 0.155 | 1.138 |
| NotFound | −0.027 | −0.278 | −0.382 | 0.395 | 0.038 | 0.019 | 0.004 | −0.32 | −0.215 | 0.129 | −0.923 | 0.1 | 0.127 |
| NotFound | 0.761 | −0.346 | −0.078 | −0.055 | 0.537 | 0.177 | 1.525 | 0.002 | −0.046 | 0.885 | −0.186 | 0.244 | 0.708 |
| NotFound | 0.5185 | 0.0855 | 0.0995 | −0.1105 | 1.0935 | 0.8805 | 1.1325 | 1.3605 | 1.2815 | 0.4705 | 1.1895 | 0.7725 | 0.7275 |
| NotFound | 1.206 | −1.011 | 0.07 | −0.869 | −0.169 | 1.53 | 1.727 | 2.037 | 1.547 | −0.087 | 0.225 | 0.265 | 0.054 |
| NotFound | 0.293 | 0.544 | 0.045 | 0.204 | 0.051 | −0.178 | −0.5 | 0.477 | 0.489 | 0.707 | −0.684 | −0.324 | −0.514 |
| NotFound | 0.0855 | 0.2435 | 0.5525 | −0.3205 | −0.3295 | 0.0095 | 0.0575 | 0.1835 | 0.1455 | −0.0845 | −0.1505 | −0.4575 | 0.5595 |
| NotFound | 1.1785 | 1.8125 | 0.1705 | −0.8905 | −1.3945 | 1.3185 | 4.2235 | 2.3375 | 2.3505 | 1.1945 | −1.9145 | 1.3925 | 2.7955 |
| NotFound | −0.039 | 0.784 | 0.047 | 0.165 | 0.221 | 0.023 | −0.143 | 0.478 | 0.464 | 0.423 | −0.421 | −0.121 | 0.166 |
| NotFound | 0.5545 | −0.1375 | −0.2726 | 0.0705 | −0.6315 | −0.0725 | −0.0385 | −0.3015 | 0.0545 | −0.0225 | −0.4565 | −1.1545 | −0.5015 |
| NotFound | 0.386 | 0.354 | −0.927 | 2.797 | 3.677 | 0.976 | −1.039 | 3.051 | 2.683 | 3.473 | −0.637 | 0.345 | 1.796 |
| NotFound | 0.449 | −0.283 | 0.331 | −0.862 | −1.172 | −0.844 | 1.356 | 2.066 | 2.076 | 1.501 | 0.302 | −0.156 | −0.55 |
| NotFound | −0.4115 | −0.1405 | −0.3945 | −0.9535 | −0.7665 | −0.2285 | 0.4235 | 0.4885 | 0.5305 | −0.5145 | 0.5515 | −1.8085 | −0.8745 |
| NotFound | −0.609 | −0.834 | 0.295 | −0.322 | 0.183 | 0.299 | −0.098 | 0.05 | 0.146 | −0.256 | −0.652 | 0.148 | −0.567 |
| NotFound | 0.2695 | −0.7835 | 0.5585 | −1.1505 | −1.2465 | −1.0325 | 2.0825 | 1.4235 | 1.8695 | 0.9595 | 0.3275 | −0.4145 | −0.8495 |
| NotFound | −0.027 | −0.391 | 0.401 | −0.038 | −0.172 | −0.062 | −0.818 | 0.503 | 0.569 | −0.899 | 0.818 | −0.708 | 0.05 |
| NotFound | 0.071 | 0.289 | 0.353 | 0.278 | 0.238 | 0.299 | 0.502 | −0.083 | −0.223 | 1.071 | 0.388 | 0.28 | 0.381 |
| NotFound | −0.1205 | −0.7995 | −0.5385 | 0.0965 | −0.4985 | −0.4965 | −0.3515 | 0.8345 | 0.9715 | −0.1715 | −0.3945 | 2.7085 | 1.9275 |
| NotFound | 0.9615 | 0.3175 | 0.0215 | 0.1155 | −1.1405 | −0.0735 | 2.5005 | 2.2305 | 2.2235 | 1.4845 | 1.1765 | −1.0815 | 0.0405 |
| NotFound | 1.4985 | −1.1015 | 0.0425 | −0.7345 | −0.3445 | −0.0895 | 3.1135 | 1.0875 | 1.0895 | 0.5375 | −0.1125 | −0.6105 | −0.9685 |
| NotFound | 0.4795 | −0.5655 | 0.4045 | −0.8375 | −0.9995 | −0.7375 | 3.5505 | 1.6305 | 1.6565 | 1.2455 | 0.2795 | −0.1495 | −0.7675 |
| NotFound | 0.2046 | 0.6995 | −0.3515 | 0.6175 | 0.8975 | −0.2655 | −0.1045 | 0.7975 | 0.8355 | 1.1005 | 1.0725 | 0.4865 | 0.1895 |
| NotFound | −0.033 | −0.133 | 0.381 | −0.648 | −0.858 | −0.731 | 2.552 | 1.397 | 1.298 | 1.147 | 0.435 | 0.023 | 0.178 |
| NotFound | 0.8216 | 0.1405 | 0.9265 | −0.0435 | −0.4855 | −0.7275 | 3.2185 | 1.9985 | 1.9595 | 1.7805 | 0.2075 | −1.9945 | −1.4105 |
| NotFound | 0.1015 | −1.7705 | −0.6425 | −0.4335 | −0.2185 | −0.1175 | 0.0675 | −0.6215 | −0.4255 | −0.7795 | 0.5015 | −0.8115 | −0.2715 |
| NotFound | −0.3715 | −0.8975 | 1.5145 | −0.6395 | −0.8435 | −0.5275 | 4.9095 | 1.2885 | 1.2845 | 1.3415 | 0.9545 | −0.2885 | 0.0975 |
| NotFound | 1.3955 | 0.1015 | 1.0065 | 0.8025 | 0.4785 | 0.6825 | −0.0115 | 0.9675 | 0.9425 | 0.0705 | 0.7775 | 0.3735 | −0.0135 |
| NotFound | 0.871 | 0.575 | 1.063 | −0.444 | −0.514 | −0.59 | −1.042 | 1.343 | 1.288 | −0.957 | −1.085 | 0.003 | −0.174 |
| NotFound | −0.255 | −0.6 | −0.132 | −0.078 | 0.2 | −0.114 | 0.242 | −0.095 | 0.194 | −0.25 | −0.046 | 0.105 | 0.032 |
| NotFound | −0.731 | −0.284 | −0.513 | −0.119 | −0.438 | −0.03 | −0.476 | 0.252 | 0.31 | −0.243 | −0.428 | 2.533 | 1.615 |
| NotFound | 0.5755 | −0.5855 | 0.4275 | −1.0015 | −0.9945 | −0.8695 | 1.8405 | 1.5875 | 1.5225 | 1.0685 | 0.3655 | −0.2225 | −0.7315 |
| NotFound | −0.0005 | −0.7725 | −0.2895 | 0.3385 | −0.0455 | 0.2165 | 1.3295 | −0.0315 | 0.1315 | −0.0595 | 0.0845 | −0.0745 | 0.3465 |
| NotFound | −0.547 | −0.287 | −0.058 | −0.054 | −0.27 | −0.214 | −0.228 | −0.371 | −0.482 | −0.517 | 0.073 | 0.083 | 0.202 |
| NotFound | 0.2075 | −0.2845 | −0.0775 | 0.3035 | 0.0145 | 0.5395 | 0.3745 | −0.2805 | −0.6355 | −0.1475 | −0.1985 | −0.0705 | −0.1045 |
| NotFound | 0.8476 | 0.1705 | 0.3615 | −0.1965 | 1.1135 | 0.8585 | −0.9435 | 0.8935 | 0.8425 | 0.1445 | 2.0605 | −1.0305 | −0.5735 |
| NotFound | −0.7855 | 0.4355 | −0.0605 | 0.3575 | 0.6705 | 0.1055 | −0.1485 | −0.2765 | −0.1895 | −0.0495 | 0.1975 | −0.6555 | 0.2795 |
| NotFound | 0.2485 | −0.5115 | −0.1795 | −0.0715 | 0.7275 | −0.1485 | 0.1595 | 1.2275 | 1.2605 | 0.4495 | 1.1105 | 1.0395 | −0.2365 |
| NotFound | 0.88 | −0.48 | −0.034 | 0.909 | 0.546 | 0.65 | −0.687 | 0.29 | 0.196 | −0.269 | −0.594 | 0.104 | 0.869 |
| NotFound | 0.988 | 0.766 | 0.523 | 0.691 | 1.146 | −0.299 | 1.001 | 0.273 | 0.306 | 0.067 | 0.54 | 1.181 | −0.526 |
| NotFound | −0.0375 | 0.2385 | −0.0685 | 0.0455 | −0.1475 | −0.8045 | 1.6675 | 1.1725 | 1.3195 | −0.4555 | 0.7275 | 0.0015 | 0.3765 |
| NotFound | −0.2245 | 0.4205 | −0.1715 | 0.1445 | 0.3755 | 0.0595 | −0.1495 | 0.7165 | 0.7605 | 0.7445 | −0.2995 | 1.0785 | 0.0105 |
| NotFound | 1.1515 | 1.5365 | 1.5885 | −0.0755 | 0.1935 | −0.2725 | 1.5295 | 2.3345 | 2.2205 | 1.7825 | −0.2605 | 1.4485 | 0.6715 |
| NotFound | 0.8075 | 1.2485 | 0.6515 | 0.2665 | 2.0645 | 0.6025 | −0.8455 | 0.5685 | 0.5305 | 0.2575 | 0.1935 | 0.3855 | 0.4625 |
| NotFound | 1.742 | −1.029 | 0.591 | −0.223 | 1.617 | 0.862 | −1.629 | 0.047 | 0.094 | 0.974 | −2.922 | −0.952 | 0.927 |
| NotFound | 4.505 | −1.021 | 0.95 | 2.258 | 2.017 | 1.066 | −0.72 | 0.833 | 0.68 | 0.383 | −0.532 | −0.036 | 4.825 |
| NotFound | 0.1255 | −0.3505 | 0.1835 | −0.3175 | −0.3775 | −0.1585 | −0.3885 | 0.0895 | −0.1035 | −0.3215 | −0.0185 | 0.3265 | 0.1555 |
| NotFound | −0.102 | 0.268 | −0.039 | 0.72 | 0.048 | −0.313 | 1.282 | 1.643 | 1.725 | 1.131 | 3.028 | −0.343 | −1.277 |
| NotFound | 0.455 | 0.032 | −0.497 | −0.369 | −0.284 | −0.206 | −0.039 | 0.879 | 1.001 | −0.085 | 0.643 | −0.656 | −0.222 |
| NotFound | 2.2025 | −0.9505 | 1.9955 | 0.4225 | −0.2855 | 0.8495 | −0.3515 | −0.2485 | 0.2035 | 0.2115 | −0.1505 | 2.1095 | −0.5445 |
| NotFound | 0.235 | 0.447 | −0.126 | 0.228 | 0.486 | 0.099 | 0.062 | 0.081 | −0.044 | 0.277 | −0.24 | −0.452 | −0.05 |
| NotFound | 1.859 | −0.061 | 0.114 | −1.164 | −1.217 | −0.432 | 3.889 | 1.258 | 1.238 | 0.538 | −0.124 | −0.728 | −1.079 |
| NotFound | 0.6485 | 0.3865 | 0.2545 | −0.9125 | −0.6675 | 0.5415 | 1.2815 | 1.7595 | 1.3875 | 1.2045 | 1.1165 | 0.4145 | −0.4005 |
| NotFound | 0.338 | −0.231 | −0.173 | 0.434 | 0.601 | 0.752 | 1.681 | 0.32 | 0.29 | 0.156 | −0.309 | 1.658 | 0.594 |
| NotFound | 0.6485 | 0.1945 | −0.4745 | 0.7195 | −0.0465 | 0.4965 | 0.0535 | −0.5795 | −0.4995 | −0.8585 | −0.2585 | 0.3835 | 0.0575 |
| NotFound | 1.3315 | −0.4015 | 0.0985 | −0.1525 | 0.1975 | 0.0865 | −0.2885 | −0.4895 | −0.3815 | −0.3815 | −0.0895 | −0.3035 | −0.2855 |
| NotFound | 0.47 | −0.587 | 0.647 | −0.952 | −1.048 | −1.054 | 2.294 | 1.678 | 1.702 | 1.137 | 0.134 | −0.415 | −0.831 |
| NotFound | −0.3395 | −0.5985 | −0.3055 | −0.4755 | 0.0375 | −0.4055 | 0.2015 | −0.1635 | −0.2465 | 0.3235 | 0.5315 | −1.2935 | 1.0235 |
| NotFound | −0.089 | −0.022 | −0.175 | 0.018 | −0.287 | −0.315 | 0.686 | 0.097 | 0.055 | 0.73 | −0.11 | −0.658 | −0.306 |
| NotFound | 0.289 | 0.587 | 0.275 | 0.166 | 0.284 | 0.914 | −0.106 | 0.623 | 0.748 | 0.191 | 0.42 | 1.147 | 0.315 |
| NPDC1 | −0.4715 | −0.3165 | 0.4055 | 0.2935 | −0.4135 | 0.2185 | −0.1385 | −1.0615 | −0.8476 | −0.5725 | −0.9075 | −0.1765 | −0.5345 |
| NPEPPS | 0.197 | 0.172 | 0.555 | 0.391 | 1 | −0.119 | 0.391 | 0.496 | 0.527 | 1.222 | −0.974 | 0.485 | 0.856 |
| NPY | 0.87 | 0.434 | 0.05 | −0.096 | −0.676 | 0.464 | −0.016 | −0.096 | −0.096 | −0.028 | −0.288 | −0.833 | −0.748 |
| NQO3A2 | 1.6 | 0.872 | 1.654 | 0.011 | 0.52 | 1.164 | 0.486 | 1.452 | 1.344 | 1.344 | 1.526 | 0.821 | 2.611 |
| NRAS | 0.249 | 1.887 | −0.722 | 0.515 | 1.621 | 0.89 | −0.355 | −1.149 | −1.172 | 1.71 | −2.485 | 1.01 | 0.46 |
| NRIP1 | 0.5015 | 0.0615 | −0.1515 | 0.8505 | 0.0085 | −0.3325 | 1.2165 | 0.9705 | 0.9095 | 1.2115 | −0.7355 | 1.0785 | 0.9765 |
| NTN4 | −0.2065 | −0.6045 | 0.0285 | −0.8605 | −0.4805 | −0.4005 | −0.1715 | −0.1645 | −0.1605 | −0.1615 | 0.0635 | 0.6035 | −0.0165 |
| NUCB2 | 0.0045 | 0.9525 | 0.3525 | 0.5145 | 0.3755 | 0.2165 | 0.5785 | 0.4255 | 0.3985 | 0.9875 | 0.8815 | 0.4815 | 0.4325 |
| NUDT5 | −0.465 | −0.348 | −0.406 | −0.562 | −0.846 | −0.138 | 0.367 | 0.846 | 0.777 | 0.378 | 0.394 | 0.161 | 0.685 |
| NUDT6 | 0.033 | 0.201 | −0.44 | −0.114 | 0.054 | −0.212 | −0.314 | −0.111 | −0.233 | 0.177 | 0.07 | −0.549 | −0.379 |
| OCIAD2 | −0.1925 | −0.5645 | 0.3415 | 0.0145 | 0.0276 | −0.0885 | −1.4615 | −0.5895 | −0.5495 | 0.0815 | −1.3245 | 0.6005 | −0.6185 |
| OFD1 | 0.1495 | 0.0485 | −0.4435 | 0.0535 | 1.1285 | 0.6035 | −0.4515 | −0.5135 | −0.4515 | −0.0045 | 0.3035 | 0.1735 | 1.4415 |
| OGFRL1 | −0.286 | 0.201 | −0.171 | −0.194 | −0.025 | −0.267 | −0.311 | 0.337 | 0.391 | −0.307 | 0.148 | −0.06 | 0.46 |
| OPLAH | 0.0345 | 0.0775 | −1.4525 | 0.5105 | 0.0425 | 0.2925 | 0.8015 | −0.7985 | −0.7215 | −0.7475 | −1.0775 | 0.1335 | −1.2445 |
| ORMDL3 | 1.476 | 0.007 | 0.855 | 0.537 | 0.164 | 0.108 | 1.288 | 3.813 | 3.737 | −0.367 | 1.23 | −1.459 | 1.084 |
| OSBPL3 | 0.27 | −0.064 | −0.201 | −0.032 | 0.164 | −0.109 | −0.205 | 0.043 | −0.135 | 0.119 | −0.218 | −0.624 | −0.581 |
| OSR2 | −0.5945 | −0.0325 | −0.3575 | 0.0865 | −0.3225 | −0.1655 | 0.1725 | 0.8045 | 0.7515 | 0.2655 | −0.3335 | 1.5125 | 0.2675 |
| OSTF1 | −0.347 | −0.666 | −0.092 | 0.344 | 0.325 | −0.033 | 0.33 | 0.347 | 0.345 | 0.939 | −0.545 | −0.643 | −0.161 |
| P2RX1 | −0.9015 | −0.5995 | −0.3035 | −0.0035 | −0.5565 | −0.7905 | −0.0205 | 0.3495 | 0.5815 | 0.7655 | −1.0595 | 1.7715 | 0.7345 |
| P2RY2 | 0.144 | −0.517 | −0.442 | 0.151 | −0.094 | 0.159 | −0.651 | −0.114 | −0.105 | −0.372 | −0.576 | −0.854 | −0.193 |
| PACAP | 0.576 | −0.671 | 0.354 | −0.159 | −0.368 | 0.21 | 2.056 | 0.105 | 0.154 | −0.541 | 0.51 | 0.373 | 0.243 |
| PADI2 | 0.0755 | −0.9325 | −0.6085 | −0.0145 | −0.3155 | 0.7485 | −0.2525 | −0.5445 | −0.3645 | 0.0905 | −0.8875 | −1.7105 | −1.1576 |
| PAPD5 | 1.28 | −0.394 | −0.028 | −0.47 | −0.082 | −0.112 | 0.234 | 0.604 | 0.624 | −0.14 | 0.261 | −0.071 | 0.216 |
| PAPSS2 | −0.494 | −0.547 | −0.117 | −0.274 | 0.103 | 0.147 | −0.28 | 0.206 | 0.179 | −0.203 | −0.581 | −0.454 | −0.105 |
| PARD8A | −0.458 | −0.693 | −0.722 | 0.002 | −0.42 | −0.216 | 0.037 | 1.91 | 1.839 | 0.999 | 1.456 | 0.437 | 0.583 |
| PARP1 | 0.317 | 0.492 | −0.41 | 0.304 | 0.118 | 0.24 | 0.639 | 1.009 | 0.972 | 0.174 | 1.312 | −0.158 | 0 |
| PARP9 | −0.1895 | −0.2815 | 0.5505 | 0.7765 | 1.1115 | 1.2775 | −0.6975 | 2.3045 | 2.3245 | 0.0515 | −0.4595 | −1.5205 | 1.4425 |
| PAXIP1L | −0.1745 | −0.1715 | −0.0605 | 0.1035 | 0.2375 | −0.0275 | −0.8345 | −0.6265 | −0.6555 | −0.8945 | −0.0635 | −0.6505 | −1.1855 |
| PBX3 | 0.2215 | 0.1815 | 0.2325 | 0.8335 | 0.0085 | 0.1695 | −0.5765 | −0.8095 | −0.7135 | −1.0055 | 0.7505 | −0.4925 | −0.6885 |
| PCDHB11 | −0.768 | 0.711 | −0.296 | −0.814 | 0.302 | −0.754 | −1.274 | −1.089 | −1.1 | −1.194 | −1.042 | −0.921 | −1.07 |
| PCDHB16 | −0.578 | 0.18 | 0.122 | 0.594 | 1.738 | 0.23 | −0.138 | −0.011 | −0.021 | 0.592 | −0.898 | −0.143 | 0.538 |
| PCDHB2 | 0.2505 | −0.0165 | −0.4885 | −0.3125 | −0.5115 | −0.5745 | −0.3625 | 0.0195 | 0.1305 | −0.1385 | 0.0155 | −0.0375 | 0.1105 |
| PCM1 | −0.4925 | −0.2715 | −0.4985 | −0.0765 | −0.0085 | −0.0155 | −0.1605 | 1.0905 | 0.8865 | 0.1985 | −0.2615 | 0.5815 | 0.0905 |
| PCNA | 0.237 | 0.458 | 0.472 | 0.154 | −0.486 | −0.156 | 0.067 | 0.372 | 0.409 | 0.461 | −0.239 | 0.24 | −0.205 |
| PDC01 | −1.4465 | 1.0445 | −0.1175 | −0.0025 | 0.7285 | 1.3435 | −1.5225 | 1.2255 | 1.0905 | 0.4345 | −2.6915 | 2.7505 | 1.5365 |
| PDC010 | −0.052 | −0.445 | 0.343 | −0.027 | −0.498 | 0.205 | −0.047 | −0.236 | −0.157 | 0.194 | 0.224 | −1.269 | −0.357 |
| PDCD4 | 0.1775 | 0.1065 | −0.2005 | −0.5225 | −0.4995 | −0.6725 | 0.5215 | −0.3845 | −0.3505 | −0.0855 | 0.6155 | 0.2645 | 0.4145 |
| PDCD5 | 0.7695 | 1.5945 | 0.2925 | 1.0865 | 1.9075 | 0.2885 | −0.5535 | −0.9605 | −0.7425 | −0.9265 | 0.7995 | −1.3585 | −1.3825 |
| PDCD8 | −0.1025 | 0.4015 | −0.1695 | −0.2465 | −0.3425 | −0.3705 | 0.3225 | −0.3455 | −0.4475 | −0.3855 | −1.0785 | −0.1435 | −0.2115 |
| PDE4DIP | 0.2845 | 0.7905 | 1.3495 | 0.1885 | 0.3355 | −0.5125 | −0.3875 | −0.6335 | −0.6705 | 0.7535 | −0.5065 | 1.6555 | −0.3965 |
| PDHA1 | −0.672 | −1.899 | −1.5 | −0.795 | 1.99 | 0.889 | −1.615 | −0.374 | −0.311 | −2.445 | −1.003 | −1.979 | 2.661 |
| PECI | 0.5435 | 0.2185 | 0.9465 | −0.0345 | 0.0565 | 0.2085 | 0.4075 | 0.2445 | 0.2685 | 0.1425 | 0.0685 | 1.9305 | 1.3485 |
| PEG10 | −0.0385 | 0.5405 | −0.3315 | −0.0605 | 0.3095 | −0.2215 | −1.8875 | −0.8425 | −0.7685 | −0.4675 | −0.0275 | 0.4365 | 0.2955 |
| PER2 | 0.142 | 0.218 | 0.364 | −0.125 | 0.091 | −0.028 | 0.252 | 0.218 | 0.266 | −0.065 | 0.367 | 0.997 | 0.092 |
| PERP | 0.1375 | 0.5125 | 0.2875 | 0.4015 | 1.1955 | 0.0965 | 0.5185 | −1.0805 | −1.0645 | −0.0675 | 0.1315 | −0.2285 | 0.5805 |
| PEX1 | −0.328 | −0.698 | −0.416 | −0.647 | −0.107 | −0.33 | −0.26 | −0.237 | −0.104 | 0.05 | 0.499 | −0.57 | −0.44 |
| PFAAP5 | −0.3775 | 0.3765 | −0.8135 | 0.5945 | −0.5055 | 0.2485 | −0.8405 | −0.1775 | 0.0995 | −0.3395 | 0.4405 | 0.4475 | 0.0195 |
| PFDN2 | 0.488 | 0.051 | 0.319 | −0.588 | 0.206 | −0.478 | 0.686 | 0.378 | 0.483 | 0.202 | 0.061 | 0.131 | 0.201 |
| PGM1 | 0.8975 | 0.1395 | −0.0765 | −0.0105 | 0.4395 | −0.1695 | 0.0455 | 0.6085 | 0.6865 | 0.3685 | −0.1385 | 0.0995 | 0.4455 |
| PGR1 | −1.8355 | −2.6805 | −1.1795 | −0.7785 | −0.8585 | −0.7505 | −1.0735 | 0.4725 | 0.5175 | −1.5885 | −1.3145 | 0.6765 | −1.4865 |
| PHAX | 0.657 | −0.259 | 0.015 | −0.408 | −0.586 | −0.839 | 0.85 | −0.114 | −0.309 | 0.128 | 0.189 | 0.468 | 0.68 |
| PHGDH | 0.0445 | −0.2335 | 0.2195 | 0.9655 | 1.4745 | 0.2205 | 1.7465 | 0.9155 | 1.1145 | 1.0585 | −0.1645 | 0.7655 | 1.2645 |
| PHYH | 0.1025 | 0.2975 | 0.0215 | 0.5755 | −0.1045 | −0.3405 | 1.0085 | 0.1495 | 0.1095 | 0.6375 | 0.3935 | 0.4785 | 0.3995 |
| PI85PA | −1.2775 | −0.3425 | −0.7055 | −0.4015 | −0.6045 | −0.4195 | −0.8315 | 0.3765 | 0.5385 | −0.5595 | −0.5495 | 1.9235 | 1.3685 |
| PIGT | −0.184 | 0.004 | 0.283 | −0.037 | −0.356 | −0.188 | 0.258 | 0.861 | 0.751 | 0.304 | 0.171 | 2.512 | 1.073 |
| PIM2 | −0.3725 | −0.5735 | −0.7745 | 0.0805 | −0.3735 | −0.2415 | −1.0165 | 0.9855 | 1.0355 | 1.0495 | −0.6185 | −0.3765 | −1.2125 |
| PISD | 0.43 | −1.111 | −0.79 | 0.961 | −0.105 | 0.979 | −1.402 | 1.382 | 1.459 | 1.441 | −1.595 | 4.594 | 3.69 |
| PITPNC1 | −0.7945 | −1.8835 | −0.9545 | −0.9415 | −0.7725 | −0.4995 | −2.3855 | −0.7085 | −0.4345 | −0.9035 | −0.0955 | 0.4955 | 0.0585 |
| PKIB | 1.358 | 0.511 | −0.095 | 0.306 | −0.07 | −0.079 | 0.506 | 0.564 | 0.546 | 0.884 | −0.204 | 0.392 | 0.206 |
| PKMYT1 | −1.262 | −0.103 | −0.031 | −0.224 | −1.245 | 1.12 | 0.328 | 1.811 | 1.747 | −0.83 | −0.306 | 0.998 | 0.11 |
| PLA2G4B | 1.2105 | 0.7095 | −0.2175 | −0.3645 | −0.4145 | −0.0555 | 0.4715 | −0.7315 | −0.6625 | −0.0495 | −0.3775 | 0.0655 | −1.0815 |
| PLAC8 | −0.589 | 0.12 | −0.717 | 0.15 | −0.25 | −0.14 | −0.14 | −0.44 | −0.52 | −0.09 | −0.42 | 0.104 | −0.492 |
| PLCB1 | 0.8595 | −0.2595 | −0.6485 | 0.2085 | 0.0815 | 0.3705 | 0.2065 | −0.5395 | −0.2275 | 0.2435 | 0.8245 | −1.3485 | −0.5725 |
| PLCXD1 | 0.381 | −0.393 | −0.564 | 0.342 | 0.336 | −0.129 | −0.756 | −0.753 | −0.641 | −0.464 | −0.267 | −0.944 | −0.525 |
| PLEK2 | 0.2095 | −0.4885 | 0.0275 | −0.0095 | 0.1405 | 0.2265 | −0.2525 | 0.0405 | 0.0685 | 0.3125 | −0.0835 | −0.6815 | −0.9605 |
| PLO0 | −0.0645 | −0.0825 | 0.3355 | −0.1375 | 0.1005 | 0.3305 | 0.0995 | 2.3845 | 2.4855 | 0.8995 | 0.1975 | 1.1495 | 0.8785 |
| PNAS-4 | 0.6225 | 0.4325 | −0.6405 | 0.5505 | 0.1855 | −0.1665 | −0.1115 | 1.6805 | 1.6075 | −0.3555 | −0.4835 | −0.4605 | −1.1675 |
| PNLIPRP2 | 0.0555 | −0.0345 | 0.2225 | 0.8075 | −0.0495 | −0.5845 | −0.3295 | 0.3595 | 0.3115 | 0.5875 | 0.8115 | 0.0275 | 1.1495 |
| PNMA3 | −0.1895 | −0.2915 | 0.1305 | −0.0295 | 0.0955 | −0.3195 | −0.4085 | 0.2155 | 0.3705 | 0.2535 | 0.2115 | −0.2745 | −0.0025 |
| POLB | 0.18 | −0.16 | −0.196 | −0.315 | −0.406 | −0.133 | 0.246 | 0.478 | 0.453 | −0.02 | −0.271 | −1.136 | −0.394 |
| POLR2F | 1.0675 | 0.3855 | −0.4295 | 0.1035 | 0.0385 | 0.2225 | −0.1115 | −0.5625 | −0.3855 | −0.3405 | −0.3625 | 1.4305 | 1.2285 |
| POLR2H | 0.2375 | 0.1555 | −0.1545 | −0.1495 | −0.1015 | 0.0925 | −0.0035 | 0.5005 | 0.5465 | 0.3945 | 0.2075 | −0.3185 | −0.0575 |
| POMC | −0.014 | −0.018 | −0.032 | −0.629 | −0.199 | −0.521 | 0.141 | 0.271 | 0.476 | 0.335 | 0.343 | 0.151 | 0.091 |
| POP4 | 0.353 | 1.185 | 0.342 | 0.472 | 0.912 | 0.156 | −0.816 | 0.008 | −0.002 | 0.002 | −0.011 | 0.011 | −0.432 |
| POP7 | −0.038 | −0.136 | −0.176 | −0.258 | −0.206 | −0.348 | 0.736 | −0.123 | −0.081 | −0.293 | −0.281 | −1.133 | 1.142 |
| PP1057 | −0.6775 | −1.4915 | 0.1325 | 0.3045 | 0.9315 | −0.1325 | 0.2355 | −0.1835 | −0.1865 | 0.6755 | −0.8715 | 0.6185 | 0.5095 |
| PPFIA1 | 0.342 | −0.518 | −0.895 | −0.152 | −0.139 | −0.379 | 0.248 | 0.168 | 0.131 | 0.231 | 0.157 | −0.094 | −0.223 |
| PPFIBP2 | 0.3735 | 0.3045 | −0.1376 | 0.0555 | 0.0275 | 0.2435 | −0.0285 | −0.0235 | 0.0035 | 0.0275 | 0.0165 | −0.3195 | −0.1005 |
| PPGB | −1.2545 | 1.1285 | 1.4155 | −0.4995 | −1.3555 | −0.1545 | −0.0945 | −0.5415 | −0.6395 | −0.4635 | −0.9255 | −0.5265 | −0.7125 |
| PPHLN1 | 0.2135 | −0.2525 | 0.0045 | 0.4035 | −0.2335 | −0.1575 | 0.1885 | 0.2905 | 0.3325 | 0.2915 | 0.1585 | −0.7385 | −0.3775 |
| PPIF | −1.2626 | 0.0115 | −0.0025 | 0.2455 | 0.6575 | −0.1335 | 0.4425 | −0.6675 | −0.4765 | 0.3245 | −0.1625 | 0.0505 | −0.1945 |
| PPIL1 | −0.2945 | −0.1215 | −0.3225 | −0.4375 | 0.3115 | 0.3525 | −0.8905 | −0.4905 | −0.5835 | −0.7865 | 0.2265 | 0.7785 | 0.4345 |
| PPOX | 1.132 | 1.688 | 0.483 | 2.785 | 0.94 | 0.452 | −0.375 | −0.679 | −0.606 | 2.553 | −0.004 | 0.048 | 0.346 |
| PPP1R16 [symbol] | −0.032 | −0.173 | −0.562 | 0.228 | 0.064 | 0.048 | −0.033 | −0.205 | −0.19 | 0.084 | −0.831 | −0.035 | −0.067 |
| PPP1R3C | −1.561 | 0.31 | 0.168 | 0.018 | 0.06 | 0.157 | 1.204 | −0.234 | −0.229 | 0.593 | −1.71 | −0.003 | 0.458 |
| PPP2R2D | −0.8945 | −2.2185 | −0.2095 | 0.4425 | 0.1965 | −0.2495 | −0.2805 | 1.1735 | 1.1475 | 4.6205 | 0.0075 | −0.7705 | −0.9385 |
| PPP2R5A | −0.934 | −2.464 | −0.778 | −0.68 | −0.785 | −0.481 | −0.947 | 0.224 | 0.314 | −0.69 | 0.302 | 0.262 | −0.206 |
| PRAME | 0.2985 | 0.1675 | 0.1685 | −0.1045 | 0.3495 | −0.0385 | −0.4285 | −0.1085 | −0.1145 | −0.0065 | −0.1945 | −0.2625 | 0.0975 |
| PRC1 | −0.441 | 0.069 | 0.103 | −0.317 | −0.218 | 0.249 | 2.935 | 0.052 | 0.312 | −0.019 | −0.184 | −0.104 | −0.112 |
| PRIM2A | −1.9135 | −0.4115 | 0.6205 | −0.2505 | −0.7895 | 0.0085 | −0.6675 | 0.5485 | 0.6835 | −0.8215 | −0.4475 | 0.5185 | −0.2455 |
| PRKACB | 0.262 | 0.307 | 0.418 | −0.085 | −0.228 | −0.002 | 1.682 | 0.246 | 0.195 | 0.028 | −0.434 | −0.333 | 0.009 |
| PRKCB1 | 0.239 | 0.323 | 0.355 | 0.128 | −0.225 | −0.034 | −0.355 | −0.047 | −0.009 | 0.444 | −0.332 | 0.331 | −0.701 |
| PRKCI | 0.165 | −0.056 | −0.682 | 0.3 | 0.398 | 0.447 | −0.206 | −0.609 | −0.527 | −0.745 | −0.39 | 0.086 | −0.598 |
| PRKRIR | 0.0025 | −0.3525 | −0.3905 | −0.0615 | 0.2585 | 0.1645 | −0.4235 | 0.5395 | 0.4205 | −0.2595 | −0.2555 | −0.1305 | −0.4405 |
| PRKWNK1 | −0.1075 | −0.2475 | 0.1805 | 0.4835 | −0.8965 | 0.3195 | −0.7265 | 0.1325 | 0.3085 | −0.5295 | −0.8805 | −1.8095 | −0.3295 |
| PRKX | −0.313 | −0.167 | 0.03 | −0.24 | −0.196 | −0.227 | −0.322 | −0.329 | −0.376 | −0.868 | −0.090 | 0.947 | 1.021 |
| PRNP | 0.2335 | −0.2695 | −0.0265 | −0.0825 | −0.1145 | −0.2215 | −0.0325 | 0.4805 | 0.3975 | 0.3475 | −0.1145 | 0.1575 | 0.5885 |
| PROL5 | 0.3055 | −0.1455 | −0.4315 | −0.0925 | −0.4865 | −0.0555 | 0.0045 | 0.4105 | 0.4585 | −0.0575 | 0.8185 | −0.1545 | 0.1375 |
| PRPF18 | −0.0115 | 0.7655 | −0.0395 | −0.0675 | −0.6085 | −0.0235 | 0.1185 | −0.9965 | −1.1805 | −0.6695 | −0.2045 | −0.3045 | 0.1775 |
| PRPSAP1 | 0.8865 | 0.8965 | −0.1855 | 0.0135 | −0.9485 | −0.0035 | −1.9815 | 1.3165 | 1.7345 | 0.9845 | 0.7445 | 0.8865 | −0.0725 |
| PRSS12 | 1.0765 | −0.2075 | 0.1745 | 2.4065 | 2.7255 | −0.0085 | −0.5415 | 0.5605 | 0.6325 | 1.3135 | −1.0425 | −2.4615 | −1.0735 |
| PRSS16 | 0.2055 | 0.0545 | −0.1355 | 0.1375 | −0.1195 | 0.0115 | −0.0035 | −0.1695 | −0.1445 | 0.5945 | −0.1415 | −0.0185 | −0.1995 |
| PSD3 | −0.1215 | −0.3585 | −1.1485 | −0.6195 | −0.4575 | −0.7675 | 0.0615 | 0.5275 | 0.4845 | −0.5525 | 0.2715 | −0.0685 | −1.1585 |
| PSMA3 | 0.403 | −0.034 | −0.59 | −0.188 | 0.058 | −0.233 | −0.317 | 0.947 | 0.853 | 0.389 | 0.591 | −0.063 | −0.399 |
| PSMA7 | 0.71 | 0.283 | 0.37 | 0.064 | −0.633 | −0.144 | −0.307 | 0.413 | 0.56 | 0.583 | 0.57 | 0.304 | 0.351 |
| PSMC5 | 0.8835 | 0.5375 | 0.5585 | 0.4135 | 0.1325 | 0.6035 | −0.0355 | 1.0965 | 1.0555 | 0.4325 | 1.3855 | −0.0525 | 0.7276 |
| PSMC6 | 0.293 | −0.083 | 0.144 | −0.506 | −0.595 | −0.398 | −0.65 | 0.73 | 0.611 | 0.193 | 0.712 | −0.487 | −0.333 |
| PSMD12 | 0.1685 | −0.0025 | −0.8955 | −0.3385 | −0.1715 | −0.1805 | −0.3795 | 0.7285 | 0.8455 | −0.0475 | 0.5745 | 0.0895 | 0.1605 |
| PSMD12 | −1.8965 | −0.8205 | 0.7585 | −1.0385 | −2.0715 | 0.9355 | −1.4625 | −2.6785 | −2.5785 | −1.4306 | 0.1155 | −1.1645 | 1.5475 |
| PSMD3 | 0.9675 | 0.4615 | 0.1305 | 0.1405 | 0.1065 | −0.3165 | −0.3515 | −0.3035 | −0.3325 | 0.7005 | −0.1075 | 0.2885 | 0.2535 |
| PSPH | 1.454 | 0.874 | 0.428 | 0.686 | 0.746 | 0.348 | −0.65 | 0.353 | 0.699 | 0.706 | 0.235 | 0.284 | 0.277 |
| PTD012 | 0.364 | −0.256 | 0.037 | 0.627 | 0.107 | 2.288 | 0.022 | 2.235 | 2.194 | 0.309 | −0.22 | −1.116 | 1.385 |
| PTGER3 | 0.708 | −0.563 | −0.478 | −0.208 | −0.127 | −0.254 | 1.311 | 1.226 | 1.027 | 0.273 | 0.67 | 0.848 | 1.189 |
| PTHLH | 0.3735 | −0.1285 | 0.3575 | 0.1735 | −0.0545 | 0.0465 | −0.2385 | 0.2315 | 0.3805 | 0.0555 | −0.0035 | −0.0355 | −0.0945 |
| PTK6 | −0.228 | −0.249 | −0.33 | −0.103 | −0.542 | 0.025 | −0.296 | 0.601 | 0.783 | 0.193 | −0.3 | 0.463 | −0.271 |
| PTPN4 | 0.294 | 0.477 | −0.343 | −0.219 | −0.004 | −0.501 | −0.497 | 0.382 | 0.589 | 0.285 | −0.948 | 0.488 | 0.154 |
| PTPN7 | −0.802 | 0.451 | 0.097 | −0.197 | −0.681 | 0.025 | 2.064 | 3.701 | 3.646 | 0.571 | 1.038 | −0.831 | 0.445 |
| PTPRF | 2.495 | −0.02 | 1.03 | 1.071 | 1.669 | 1.392 | −0.325 | 0.6 | 0.905 | 1.675 | 0.141 | −0.781 | 0.463 |
| PTPRN2 | 0.8965 | 0.2365 | −0.2905 | 0.7315 | −0.2835 | −0.3565 | −0.6975 | −0.1025 | −0.1435 | 0.2945 | −0.0695 | −0.3295 | 0.1455 |
| PTPRT | −0.5415 | −2.8095 | −1.1165 | −0.6575 | −0.4495 | −0.4145 | −1.0315 | 0.0465 | 0.2015 | −0.4565 | 0.0155 | 0.1875 | −0.5355 |
| PTS | 0.223 | 0.784 | −0.586 | −0.191 | −0.012 | −0.577 | −0.561 | 0.44 | 0.562 | 0.202 | −0.352 | −0.12 | 0.468 |
| PTTG1 | 0.8825 | −0.5275 | −0.2845 | −0.1135 | −0.3755 | −0.0575 | 1.0135 | 0.3885 | 0.2685 | 1.1395 | −0.3405 | 0.3195 | 0.5015 |
| PURB | −0.4395 | −0.7535 | −0.7325 | 0.0925 | 1.4175 | 0.4145 | −0.1185 | 0.0375 | 0.0285 | 1.3845 | 0.2335 | −0.4695 | −0.1185 |
| PXMP2 | 0.7635 | 0.3585 | −0.5005 | 0.2705 | −0.0645 | −0.5255 | −0.5465 | 0.6005 | 0.8315 | 0.0315 | 0.4665 | 0.3285 | −0.6045 |
| PYCARD | 0.122 | 0.092 | 3.755 | 1.41 | 0.051 | 0.347 | 1.045 | −1.133 | −1.017 | 1.147 | −0.224 | 0.839 | 1.761 |
| PYCR1 | −1.055 | −0.528 | −0.121 | −0.797 | −0.252 | −0.678 | −0.507 | 0.888 | 0.834 | −1.36 | −0.833 | −0.936 | −1.039 |
| QDPR | −0.4315 | 0.5115 | −0.1455 | 0.6575 | 0.2865 | 0.3745 | −0.0425 | −0.8585 | −0.4975 | 0.9425 | 0.5675 | −0.8965 | 0.5465 |
| QPRT | 3.242 | −0.185 | 0.338 | 0.189 | 0.534 | −0.309 | 1.613 | 1.105 | 1.11 | 1.405 | 2.605 | −1.063 | 4.256 |
| QSCN9 | −0.1245 | 0.7915 | 0.6995 | −0.2975 | −0.7215 | −0.8005 | −0.8126 | 0.6815 | 0.8005 | 0.7885 | −0.0635 | −0.1855 | 0.7405 |
| RAB11FIP | 0.288 | 0.028 | −0.548 | 0.097 | 0.213 | 0.028 | 0.268 | 0.236 | 0.107 | 0.453 | 0.014 | −0.18 | 0.403 |
| RAB11FIP | 0.168 | −0.242 | −0.275 | −0.002 | −0.484 | 0.351 | −0.141 | −0.502 | −0.252 | 0.007 | −0.845 | −0.232 | −0.402 |
| RAB13 | −0.602 | 0.207 | −0.284 | 0.024 | −0.016 | −0.373 | 0.183 | 0.037 | 0.038 | 1.045 | −0.339 | 1.58 | 0.24 |
| RAB27A | −0.392 | 0.047 | −0.192 | 0.289 | 0.542 | 0.147 | −0.003 | −0.638 | −0.688 | 0.096 | −1.492 | −0.614 | 0.371 |
| RAB30 | 0.3335 | −0.0876 | −0.5305 | −0.2885 | −0.5185 | −0.1075 | 0.0325 | 0.2435 | 0.2385 | 0.0395 | −0.1085 | −0.5535 | −0.0435 |
| RAB34 | 1.696 | 0.089 | 0.182 | −0.027 | −0.538 | −0.017 | −0.682 | −0.788 | −1.283 | −0.45 | 1.261 | −0.858 | 1.06 |
| RAB35 | 0.105 | 0.173 | 0.001 | 0.053 | −0.102 | −0.291 | 0.047 | 0.609 | 0.541 | 0.132 | −0.27 | −0.575 | −0.484 |
| RAB39B | −0.0005 | −0.7035 | −0.2145 | 1.1465 | −0.4925 | 0.8595 | 0.0695 | −0.6505 | −0.8945 | 0.4995 | −0.2355 | 0.8965 | −0.3715 |
| RAB3A | −0.872 | 0.437 | −0.802 | −0.829 | 2.25 | 0.896 | 1.512 | −0.987 | −1.067 | 0.703 | 1.483 | 0.094 | 0.441 |
| RAB3IP | 0.365 | 0.088 | −0.073 | 0.188 | 0.252 | −0.046 | −0.095 | 0.451 | 0.419 | −0.089 | −0.078 | 0.319 | −0.527 |
| RABEP1 | −0.394 | −1.357 | −0.352 | −0.794 | −0.887 | 0.227 | −1.714 | −0.584 | −0.503 | −0.921 | −0.084 | 0.071 | 0.074 |
| RABGAP1 | 0.1385 | 0.4405 | 0.2505 | 0.4515 | 0.8745 | 0.3295 | 0.4495 | 0.0025 | 0.0355 | 0.6315 | 0.2605 | 0.3095 | 0.6025 |
| RACGAP1 | −0.5175 | 1.3415 | −0.7355 | −1.1975 | −1.3846 | −0.5545 | −1.7385 | −0.6095 | −0.5465 | −0.8525 | −0.1275 | −0.0115 | −0.2395 |
| RAD17 | −0.761 | −1.085 | −0.438 | −0.625 | −0.658 | −0.169 | −1.501 | 0.255 | 0.205 | −0.345 | 0.182 | 0.577 | −0.596 |
| RAD51 | −0.343 | −0.103 | 0.342 | 0.113 | −0.154 | −0.235 | 0.384 | −0.542 | −0.548 | −0.255 | 0.284 | −0.034 | −0.213 |
| RAD51AP | −0.8395 | −1.8385 | −1.1955 | −0.2116 | −0.7415 | −0.4805 | −1.3685 | −0.8285 | −0.5855 | −0.8815 | −0.0625 | 0.0515 | −0.4925 |
| RAD52 | −0.426 | −1.106 | 0.204 | −0.576 | −1.026 | −0.016 | −2.122 | −0.716 | −0.736 | −0.706 | −0.728 | 0.035 | −0.802 |
| RAD54L | −0.5295 | 0.1075 | −0.4855 | −0.1855 | −0.2875 | −0.1555 | −0.4375 | −0.1135 | 0.0405 | −0.2005 | −0.1715 | −1.1285 | −0.4185 |
| RAMP | 0.0575 | −0.5705 | −1.3845 | −1.0055 | −1.7385 | −0.2395 | −0.3285 | 0.5825 | 0.7485 | −0.0035 | −1.9435 | 0.3105 | 1.2245 |
| RANBP1 | 0.1085 | −0.2835 | 0.0335 | −0.1675 | −0.1375 | −0.1735 | −0.1075 | −0.3605 | −0.3505 | 0.0265 | −0.8345 | 0.2795 | 0.0355 |
| RARRES1 | 0.618 | −0.35 | −0.138 | −0.353 | −0.838 | 0.848 | 0.193 | 0.63 | 0.652 | 0.991 | 0.385 | 0.671 | 0.895 |
| RBBP7 | −0.1915 | −0.6655 | −0.1505 | −0.3365 | 0.1525 | −0.1385 | 0.2605 | −0.2945 | −0.2495 | −0.0425 | 0.0635 | −0.3695 | −1.4725 |
| RBBP8 | −0.1185 | 0.2425 | 0.2405 | 0.3255 | 0.1745 | 0.1395 | 0.3475 | 0.1435 | 0.1995 | 0.3665 | −0.1265 | −0.9085 | 0.5565 |
| RBM8A | 1.774 | 0.478 | −0.707 | 0.248 | −0.448 | 1.765 | 0.31 | 0.862 | 0.711 | 0.701 | 0.442 | −0.512 | 1.236 |
| RBX1 | −0.455 | 0.774 | −0.078 | 0.278 | 0.438 | −0.012 | −0.514 | −0.156 | −0.102 | 0.01 | −0.065 | −0.045 | 0.075 |
| RDHE2 | 0.623 | 0.48 | 0.725 | −0.01 | 0.19 | −0.148 | 0.517 | 0.554 | 0.544 | 0.207 | 1.495 | −0.028 | 0.22 |
| REA | 0.08 | −0.214 | 0.021 | −0.069 | −0.449 | −0.027 | −0.078 | −0.073 | −0.2 | −0.047 | 0.601 | −0.834 | −0.133 |
| REC14 | −0.1975 | −0.4315 | −0.8355 | 0.0445 | 0.2365 | 0.3535 | −0.8445 | −0.2205 | −0.1975 | 0.3925 | −0.4905 | −0.6425 | −0.1945 |
| REG1A | −0.071 | 0.014 | 0.404 | 0.017 | 0.41 | −0.189 | 0.029 | 1.184 | 1.126 | −0.077 | 0.474 | 0.203 | 0.688 |
| RELB | 0.904 | −0.058 | −0.312 | −0.541 | 0.086 | −0.09 | 0.898 | 0.246 | 0.256 | 1.465 | −1.174 | 1.079 | 0.299 |
| REPIN1 | −0.76 | 0.556 | −0.087 | 0.002 | −0.241 | 0.006 | 0.475 | 3.85 | 3.227 | 0.801 | 0.317 | 0.831 | 2.2 |
| REPS2 | 0.1215 | −0.3935 | −0.1565 | −0.5025 | −0.4175 | −0.3185 | −0.6635 | 0.3685 | 0.4705 | −0.2915 | −0.1775 | −0.4725 | 0.0825 |
| RERG | −0.3095 | −0.2395 | −0.0485 | −0.0845 | −0.1215 | −0.0035 | −0.0285 | −0.1475 | −0.1865 | 0.5805 | −1.8225 | 0.1755 | −0.8765 |
| RFC4 | 0.1455 | −0.1765 | −0.1815 | −0.0835 | −0.7485 | −0.1215 | −0.5915 | 0.1585 | 0.1915 | 0.2075 | −0.3115 | −1.4305 | −0.6075 |
| RGS10 | 0.1515 | −0.0555 | −0.3265 | 0.0965 | −0.3825 | −0.0505 | −0.3175 | 0.8465 | 0.8645 | −0.4225 | −0.8135 | 0.1365 | 0.5865 |
| RH8DL2 | 0.687 | 0.128 | 0.187 | 0.116 | 0.355 | −0.023 | 0.929 | 0.367 | 0.361 | 0.58 | 0.02 | −0.417 | 0.4 |
| RIBC2 | 0.648 | −0.031 | 0.164 | −0.272 | −0.174 | 0.092 | 0.034 | 0.106 | 0.156 | 0.668 | 0.174 | −0.901 | −0.555 |
| RIC-8 | 0.993 | 1.323 | 0.48 | 0.543 | 0.054 | 0.438 | 0.014 | 0.492 | 0.549 | 0.707 | 2.434 | 0.293 | 4.908 |
| RIT1 | −0.379 | −0.344 | −0.535 | −0.048 | 0.31 | −0.077 | −0.015 | −0.172 | −0.103 | −0.135 | −0.358 | −0.406 | 0.221 |
| RLN2 | 0.798 | 0.409 | 0.518 | −0.43 | 0.2 | 0.089 | 0.838 | 0.315 | 0.457 | 0.414 | 0.083 | 0.358 | 0.433 |
| RNF10 | 0.3685 | 0.2355 | 0.2875 | −0.1265 | −0.2435 | −0.3675 | −0.1535 | 0.4935 | 0.5335 | 0.7515 | 0.0555 | 0.0825 | −0.2165 |
| RNF135 | −0.8375 | −0.1515 | 0.4085 | −0.3325 | −0.1895 | −0.2075 | −0.0225 | −0.6125 | −1.1515 | −1.2365 | −0.2045 | −0.1485 | −0.8185 |
| RNF7 | 0.256 | 0.163 | 0.243 | −0.008 | −0.268 | −0.102 | 0.509 | 0.277 | 0.367 | 0.184 | −0.128 | 0.348 | 0.122 |
| ROPN1 | −0.7875 | 0.5785 | 0.4045 | 1.1285 | 0.0055 | 1.6055 | −0.8845 | 1.9725 | 1.9625 | −0.4505 | −1.6115 | −1.1005 | 0.2275 |
| RPA3 | 0.0855 | −0.1845 | 0.1755 | 0.2685 | 0.3805 | 0.1795 | −0.2285 | 0.6485 | 0.7595 | 0.4715 | −0.0085 | −0.3775 | 0.0155 |
| RPESP | 0.248 | −0.328 | 0.063 | −0.354 | −0.187 | 0.089 | 0.231 | −0.258 | −0.321 | −0.142 | −0.166 | −0.327 | −0.413 |
| RPL11 | 0.119 | −0.502 | 0.012 | −0.146 | −0.064 | −0.099 | 0 | 0.03 | 0.143 | 0.558 | −0.248 | −0.158 | −0.359 |
| RPL13 | 0.273 | 0.275 | 0.985 | 0.074 | 0.334 | 0.215 | 0.167 | 0.347 | 0.289 | 0.24 | 0.228 | −1.066 | 0.025 |
| RPL13A | −0.2535 | −0.0275 | −0.2875 | −0.1695 | 0.0905 | 0.0275 | −0.4905 | 0.5405 | 0.5665 | 0.8605 | 0.1495 | −0.8285 | −0.4845 |
| RPL15 | 0.157 | −0.14 | 0.422 | −0.149 | −0.049 | 0.052 | 0.358 | 0.23 | 0.62 | −0.114 | −0.069 | −1.126 | −0.116 |
| RPL18A | 0.346 | 0.208 | −0.002 | 0.077 | 0.58 | 0.15 | −0.642 | 5.024 | 0.002 | 0.15 | 0.13 | −0.82 | −0.502 |
| RPL19 | 0.255 | −0.348 | −0.043 | −0.037 | −0.332 | 0.199 | 0.424 | 0.061 | 0.207 | 0.411 | −0.516 | −0.061 | −0.061 |
| RPL21 | 0.2045 | −0.1875 | −0.0185 | −0.1215 | −0.1555 | 0.0395 | 0.4095 | 0.1715 | 0.2625 | 0.3285 | −0.4695 | 0.1055 | −0.2135 |
| RPL23A | −0.263 | 0.295 | −0.283 | 0.282 | 0.308 | −0.141 | 0.387 | 0.135 | 0.214 | 0.969 | −0.314 | −0.221 | 0.632 |
| RPL23A | −0.4565 | −0.3945 | −0.7655 | 0.0345 | 0.1145 | 0.2285 | −0.0815 | −0.3245 | −0.2975 | 0.5075 | 0.0515 | 0.0265 | −0.1125 |
| RPL26L1 | −0.3545 | −0.7375 | −0.0755 | −0.0305 | 0.2965 | 0.2565 | −0.2685 | 0.2115 | 0.2125 | 0.7525 | 0.2205 | −0.8225 | 0.0595 |
| RPL29 | 0.3615 | −0.0385 | 0.0965 | 0.1235 | 0.0205 | −0.0645 | 0.0005 | 0.4325 | 0.2955 | 0.6395 | 0.1805 | −0.3975 | −0.1735 |
| RPL35 | 0.0625 | −0.5065 | −0.5605 | −0.1685 | −0.3085 | −0.0555 | −0.2295 | 0.1395 | 0.3326 | 0.6745 | −0.8235 | −0.0665 | −0.3505 |
| RPL35AL | −0.0185 | −2.1695 | 0.4225 | 0.5625 | 0.6855 | 0.1185 | −0.4165 | −2.7505 | −1.4555 | −1.7465 | 0.1195 | −0.4475 | −0.9195 |
| RPS16 | 0.1115 | −0.0535 | 0.4785 | 0.0505 | 0.0705 | −0.0205 | 0.2025 | 0.0385 | 0.0826 | 0.2125 | −0.0325 | −0.7265 | 0.0945 |
| RPS4Y1 | 0.0055 | −0.3095 | −0.4745 | 0.3635 | 0.0575 | 0.0995 | 0.1355 | 0.3205 | 0.3955 | −0.4205 | −0.5845 | −0.6025 | −0.5955 |
| RRAGA | 0.175 | −0.162 | 0.459 | −0.535 | −0.046 | 0.003 | 0.414 | 0.128 | 0.209 | 0.128 | −0.255 | 0.472 | 0.449 |
| RRAGD | −0.827 | 1.469 | −1.890 | −1.634 | −1.412 | −0.913 | −2.233 | −0.758 | −0.637 | −0.615 | 0.103 | −0.942 | −0.629 |
| RRM1 | 0.136 | 0.121 | 0.27 | −0.444 | 0.324 | −0.199 | −0.203 | 0.304 | 0.285 | −0.122 | 0.32 | 0.014 | −0.074 |
| RRM2 | −0.384 | 0.921 | 0.524 | 0.082 | −0.827 | 0.424 | −1.163 | 0.506 | 0.388 | 0.005 | 0.203 | −1.834 | −0.043 |
| RSAFD1 | −0.0895 | −0.3525 | −0.2535 | 0.2225 | 0.1395 | 0.2416 | −0.2725 | −0.1725 | −0.2575 | −0.0665 | 0.1605 | −0.8425 | −0.6415 |
| RSNL2 | −0.0955 | −0.0295 | −0.7865 | 0.0415 | −0.0045 | −0.1185 | −0.2845 | −0.0125 | −0.0065 | 0.1465 | 0.3815 | −0.0415 | 0.2135 |
| RSU1 | −0.073 | −0.827 | −0.753 | −0.145 | 0.213 | −0.154 | 0.288 | −0.885 | −0.605 | 0.287 | −0.216 | 0.19 | 0.701 |
| RTN4IP1 | 1.179 | 0.004 | −0.852 | 0.136 | 0.742 | 0.782 | 0.722 | 0.399 | 0.28 | 1.321 | −3.098 | −3.213 | 2.87 |
| S100A11 | 0.247 | −0.716 | −0.004 | 0.486 | 0.386 | 0.47 | 1.415 | −0.455 | −0.461 | 0.994 | −1.496 | −1.448 | 2.159 |
| S100A14 | −2.1685 | −0.4765 | −1.7285 | −0.8065 | −0.7035 | 2.9595 | 4.6365 | 0.4735 | 0.5635 | 2.0915 | −1.5595 | −1.5455 | 1.4635 |
| S100A16 | −0.1905 | 0.3225 | −0.4455 | −0.3726 | −0.2255 | −0.5245 | −0.2815 | −0.3385 | −0.2605 | 0.1325 | 0.3645 | 0.0675 | 0.1785 |
| S100A9 | 0.235 | 0.813 | 1.568 | 0.522 | 0.827 | 0.533 | −0.851 | 0.751 | 0.766 | 0.646 | −0.193 | 0.515 | −0.508 |
| SAE1 | 0.5155 | 0.0205 | −0.1315 | −0.0685 | −0.3075 | −0.4475 | 0.2175 | 0.3665 | 0.3005 | 0.5475 | −0.6655 | −0.2995 | −0.3135 |
| SALL2 | −0.288 | 0.087 | 0.022 | −0.552 | −0.421 | −0.678 | 0.605 | 0.603 | 0.666 | 0.754 | −0.08 | 0.779 | 0.391 |
| SARA1 | 0.0055 | 0.4575 | 0.0415 | 0.5555 | −0.1085 | 0.2945 | 0.0595 | 0.4475 | 1.0285 | 0.5015 | −0.3045 | −0.1205 | 0.2835 |
| SARA2 | 0.488 | −1.03 | −0.593 | 0.071 | −0.511 | 0.298 | 0.048 | −0.166 | 0.561 | 0.528 | −0.492 | −0.416 | 1.469 |
| SAS | −0.086 | 0.384 | 0.203 | 1.043 | 0.209 | 0.523 | 0.886 | −1.224 | −1.05 | −2.078 | −0.742 | −1.014 | 0.097 |
| SCIN | 0.0605 | 0.7585 | −0.7565 | −0.3975 | −0.4375 | −0.1925 | −1.3805 | 0.0195 | 0.0855 | 0.8265 | −1.2605 | 0.1845 | −0.0315 |
| SCML1 | 0.145 | 0.244 | −0.142 | 0.107 | 0.286 | 0.708 | −0.583 | −0.404 | −0.452 | −0.573 | −0.125 | 0.625 | 0.538 |
| SCPEP1 | 0.9685 | −1.2895 | −2.3165 | 1.1785 | 1.4205 | 0.2145 | −0.8935 | 0.1015 | 0.1345 | 0.0545 | −0.8045 | −0.6415 | 0.1815 |
| SCRIB | −0.884 | 0.412 | 0.859 | −0.61 | −1.114 | −0.077 | −0.792 | 0 | 0.038 | −0.663 | −0.49 | −0.009 | −0.08 |
| SDC1 | 0.161 | −0.168 | 0.036 | −0.137 | 0.308 | 0.13 | −0.465 | 0.543 | 0.665 | 0.407 | −0.011 | −0.904 | −0.553 |
| SDC3 | −0.0125 | −0.2545 | −0.2445 | 0.1625 | −0.0865 | 0.2425 | −0.0485 | −0.1955 | −0.1155 | 0.7995 | −0.2195 | 0.2765 | −0.2285 |
| SDCCAG0 | 0.2635 | 0.0055 | −0.4795 | 0.1145 | 0.0925 | −0.1185 | 0.1175 | −0.2655 | −0.2735 | −0.0235 | 0.1105 | −0.0705 | −0.2165 |
| SEC61G | 0.505 | 0.33 | 0.34 | −0.01 | −0.088 | 0.116 | 0.151 | 0.18 | 0.03 | 0.437 | −0.173 | 0.503 | 0.009 |
| SECP43 | 1.4725 | −0.3585 | −0.5235 | 0.8475 | 0.3155 | 2.0415 | 3.5275 | −0.5105 | −0.2835 | 0.7135 | −0.4585 | 2.1695 | 2.1785 |
| SEDLP | 0.399 | 0.073 | 1.33 | 1.076 | 0.611 | 1.728 | 0.586 | −0.028 | −0.016 | 1.02 | 0.184 | 0.26 | 2.379 |
| SEMA3B | 0.3825 | 0.1295 | −0.3535 | 0.1085 | 0.3455 | −0.0795 | 0.4415 | −0.1145 | −0.0135 | 0.0735 | 0.3235 | −0.4235 | 0.4255 |
| SEMA3C | −0.1825 | 0.7665 | 0.6575 | 0.4955 | −0.2185 | −0.0625 | −0.0505 | 0.4945 | 0.4335 | 0.6715 | −0.0045 | 2.2905 | 0.1565 |
| SEPW1 | 1.763 | 1.309 | 0.649 | −0.914 | −0.112 | 1.575 | −0.656 | 2.314 | 2.288 | 1.014 | −1.234 | 1.044 | 2.017 |
| SERP1 | −0.5045 | −0.3525 | 0.0315 | 1.9605 | −0.5185 | 0.6395 | −0.3395 | −1.0725 | −1.1825 | −1.2905 | −0.2055 | −1.2855 | 0.1505 |
| SERPINA [symbol] | −2.39 | 0.113 | −0.141 | −1.293 | −1.501 | 0.272 | −0.506 | −0.277 | −0.28 | −2.283 | −1.124 | −0.256 | 0.832 |
| SERPINB [symbol] | −0.635 | 0.306 | −0.097 | 0.323 | −0.093 | 0.253 | −0.369 | 0.38 | 0.313 | 0.153 | 0.141 | 0.657 | 0.643 |
| SFRP1 | 1.192 | 0.411 | 0.844 | 1.285 | 0.477 | 0.718 | −0.058 | 0.444 | 0.283 | 0.594 | 0.591 | −1.243 | −0.944 |
| SFRS6 | 0.2525 | 0.0965 | −0.1285 | −0.3525 | 0.7385 | −0.3055 | −1.7605 | −0.1825 | −0.2495 | −0.5575 | −0.3585 | −1.6465 | 0.5015 |
| SGCD | 0.6195 | 1.7385 | 2.1155 | 0.6585 | −0.0525 | 0.8725 | 2.8235 | 0.3405 | 0.2675 | 2.0525 | −0.6285 | 2.2835 | −0.2135 |
| SGCE | 0.255 | −0.001 | −0.097 | −0.406 | −0.232 | −0.28 | −0.347 | 0.142 | 0.182 | 0.258 | 0.114 | 0.403 | −0.268 |
| SH3BGRL | 1.015 | 1.172 | 0.258 | 0.045 | −0.747 | 0.5 | −0.264 | 0.553 | 0.564 | 0.838 | 1.723 | 0.173 | 1.199 |
| SHFM1 | −0.5235 | −0.7945 | −0.2755 | −0.0035 | −0.0735 | 0.2425 | −0.0795 | −0.1935 | −0.2255 | 0.2015 | −0.1885 | 0.1755 | 0.3175 |
| SIAT7B | −1.541 | −0.758 | −0.731 | −0.062 | −0.657 | 0.088 | −0.44 | 0.872 | 0.951 | −0.126 | −0.559 | 1.786 | −0.139 |
| SIGLEC12 | 0.268 | 1.12 | −0.135 | −0.564 | 0.109 | −0.464 | 0.272 | 1.161 | 1.08 | 0.151 | 0.055 | 0.024 | −0.092 |
| SIT | 0.6585 | 0.5945 | 0.7285 | 0.3875 | 0.9315 | 0.0185 | 0.8525 | 1.3005 | 1.2255 | 0.7145 | 0.2155 | 0.7245 | 0.3405 |
| SITPEC | −0.2895 | −0.2255 | −0.1025 | 0.1975 | 0.1065 | −0.1665 | 0.0665 | −0.1245 | −0.0095 | −0.2085 | 0.2665 | 0.1305 | −0.0585 |
| SKP1A | 8.0605 | 0.5675 | −0.4295 | −0.2985 | −0.1125 | −0.0075 | 0.0325 | −0.8885 | −0.9455 | −0.5375 | −0.8665 | −0.8285 | −0.9285 |
| SLC12A9 | 2.382 | 0.937 | 0.402 | −0.037 | 1.874 | 1.327 | 2.303 | 0.581 | 0.531 | −0.88 | 0.481 | 1.166 | 1.435 |
| SLC13A3 | −0.4155 | −0.0955 | 1.9035 | 0.1435 | 0.0185 | 0.7895 | −0.8685 | 0.5575 | 0.5765 | 1.4005 | −0.5815 | 0.0985 | 0.5205 |
| SLC16A6 | 2.325 | 0.285 | 2.972 | 3.628 | 0.127 | 1.971 | −1.299 | 0.518 | 0.563 | 2.984 | 0.056 | 0.388 | 2.259 |
| SLC19A2 | −0.7545 | 0.2785 | −0.2045 | −0.7995 | 0.8295 | 2.3505 | 1.4975 | 1.0215 | 0.8605 | 0.4075 | −1.3425 | 1.1645 | 3.8275 |
| SLC1A1 | −1.7095 | 0.5505 | 0.4885 | −1.6505 | 0.2265 | −0.2575 | −1.5855 | 1.1525 | 1.3845 | 0.7985 | −0.6065 | −2.2885 | −0.7415 |
| SLC1A2 | −0.3425 | 0.4775 | −0.0895 | −0.2675 | 0.1565 | −0.0635 | 0.6135 | −0.3245 | −0.3875 | 0.2075 | −0.4795 | 0.5635 | −0.1155 |
| SLC24A3 | 0.698 | −0.096 | 0.349 | 0.405 | −0.062 | −0.353 | 0.89 | 0.734 | 0.682 | 0.454 | 1.082 | −2.452 | −0.14 |
| SLC25A29 | −0.275 | −0.591 | 0.059 | −0.463 | −0.511 | −0.095 | −0.276 | 0.004 | 0.086 | 0.257 | 0.392 | −0.334 | −0.565 |
| SLC25A4 | 1.595 | 1.94 | 1.523 | 1.869 | −0.466 | 1.882 | 1.802 | 1.439 | 1.465 | 1.091 | 1.12 | 2.038 | 4.991 |
| SLC25A5 | 0.1165 | 0.3945 | −0.1515 | −0.4375 | −0.0635 | 0.2965 | 0.2785 | 0.6215 | 0.6055 | −0.0445 | −0.6235 | 0.0095 | −0.0975 |
| SLC27A2 | −0.874 | −0.042 | 0.229 | −0.525 | 0.225 | −0.272 | 0.206 | 4.053 | 3.459 | 2.083 | −0.018 | 0.071 | −0.578 |
| SLC27A5 | 0.234 | 0.231 | −0.003 | −0.497 | −0.051 | 0.353 | −1.551 | 0.655 | 0.628 | −0.178 | −0.259 | 0.066 | −0.24 |
| SLC30A8 | 1.8565 | 1.5935 | 0.3405 | 0.5615 | 0.8385 | 1.7035 | 4.0305 | 4.2505 | 4.1535 | 2.2745 | 2.5895 | 2.6075 | 3.5855 |
| SLC39A4 | 0.806 | 2.028 | 2.502 | 1.975 | 1.343 | 2.407 | −1.399 | 0.817 | 0.768 | 1.152 | −1.925 | 0.175 | −0.602 |
| SLC39A6 | 0.1065 | 0.0685 | −0.0785 | −0.1245 | −0.1645 | −0.3805 | 0.4225 | −0.3325 | −0.3175 | −0.7205 | −0.5705 | −0.0635 | 0.0735 |
| SLC40A1 | −1.0885 | −0.8155 | −0.9475 | −0.5415 | 0.0905 | −0.1245 | −1.0745 | −0.2115 | −0.2505 | 0.2035 | −0.0195 | −1.0525 | −0.0565 |
| SLC41A3 | −0.219 | 4.138 | 1.47 | 1.901 | 2.097 | 2.493 | −0.22 | −0.75 | −0.875 | 1.94 | −0.162 | −0.432 | 2.97 |
| SLC5A6 | 0.1025 | 0.8135 | −0.1555 | −0.5325 | 0.0005 | −0.0435 | 3.5895 | 2.1095 | 2.0635 | 0.2005 | 1.8865 | 0.2825 | 0.5895 |
| SLC6A4 | 2.6055 | −0.6615 | −0.1035 | 1.0165 | 3.3445 | 2.4845 | −1.8685 | 2.1165 | 2.1525 | 1.4645 | −2.3465 | −1.3585 | 2.8085 |
| SLC9A3R1 | 0.5095 | 0.0985 | 0.4565 | 0.6395 | 2.1265 | 0.7115 | −1.2905 | 0.5535 | 0.3045 | 1.2945 | −0.3875 | 1.1485 | 3.1565 |
| SLITRK6 | 0.711 | −0.101 | 0.287 | −0.001 | 0.151 | 0.105 | −0.255 | 0.382 | 0.436 | 0.228 | −0.384 | −0.524 | −0.086 |
| SMA3 | 0.734 | 0.11 | 0.021 | 0.692 | 0.261 | 0.275 | −0.678 | −0.199 | −0.206 | −0.286 | −0.655 | −0.832 | −0.885 |
| SMAD2 | 0.121 | 0.427 | −0.004 | 0.094 | 0.099 | 0.257 | 0.147 | 0.503 | 0.507 | 0.384 | −0.567 | 0.09 | 0.347 |
| SMO | 0.316 | −0.08 | −0.28 | −0.01 | −0.243 | 0.089 | 0.379 | −0.287 | −0.416 | −0.409 | −0.021 | −0.115 | −0.08 |
| SMU1 | 0.135 | 0.195 | −0.044 | −0.088 | −0.199 | 0.004 | 0.094 | 0.187 | 0.314 | 0.631 | −0.158 | −0.582 | −0.215 |
| SNPH | −0.266 | −0.179 | −0.618 | 0 | −0.325 | −0.056 | −0.857 | 0.199 | 0.152 | 0.089 | −0.051 | −0.039 | −0.356 |
| SNRPB2 | −0.4625 | 0.1825 | 0.2145 | −0.2335 | −0.1435 | −0.2135 | 0.0225 | 0.6115 | 0.6915 | 0.4685 | −0.1615 | −0.6755 | −0.0245 |
| SNRPD1 | −0.104 | −0.053 | 0.342 | 0 | −0.377 | 0.108 | −0.371 | −0.291 | −0.225 | 0.19 | −0.242 | −0.098 | 0.168 |
| SNRPD3 | 1.0155 | 0.1885 | 0.0365 | 0.6205 | −0.1275 | 0.0025 | −0.3725 | 0.0995 | 0.0215 | 0.4445 | −0.0505 | −0.9095 | 0.5135 |
| SNRPG | −1.0215 | −0.3335 | −1.5625 | −0.5335 | −0.2745 | 0.0855 | −0.6445 | −0.1145 | −0.0825 | −0.1715 | −0.9605 | 0.3905 | −0.7075 |
| SNX7 | −0.0015 | −0.0795 | 0.0155 | 1.2075 | −0.2825 | 0.8935 | 0.5005 | 0.3875 | 0.5765 | 0.3365 | −0.0965 | −0.4605 | 0.3965 |
| SO02 | −0.1905 | 0.1705 | 1.0375 | −0.4845 | 0.4595 | 0.3205 | −0.2075 | −0.2155 | −0.0595 | −0.1455 | −0.3835 | −0.8245 | 0.9525 |
| SORCS1 | −1.341 | −0.268 | −0.417 | −0.785 | −0.703 | −0.077 | −1.535 | −2.123 | −2.018 | −0.673 | −0.886 | −1.11 | 0.107 |
| SOX4 | 2.1315 | −0.0795 | 0.0105 | −0.5575 | −1.4385 | 0.3015 | −1.5175 | −0.4275 | −0.4155 | 1.3795 | −1.8105 | 0.3255 | −1.7805 |
| SOX9 | −0.877 | −1.79 | −1.381 | −1.222 | −1.082 | −0.355 | −1.78 | −0.736 | −0.677 | −0.951 | −0.273 | −0.077 | −0.359 |
| SPAG18 | 0.6855 | 0.2965 | 0.0645 | −0.0065 | −0.2555 | 0.0685 | −0.4915 | 0.7965 | 0.7425 | 0.8255 | 0.3985 | 0.1265 | −0.0985 |
| SPAG5 | 2.3135 | 0.3875 | 0.0735 | 0.0955 | 0.8955 | −0.4885 | −0.1216 | 0.4445 | 0.5005 | 1.0595 | 1.6235 | −1.2125 | 1.4965 |
| SPC18 | 0.474 | 0.008 | −0.249 | 0.186 | −0.195 | −0.092 | −0.922 | 0.38 | 0.5 | −0.284 | 0.208 | −0.609 | −0.925 |
| SPFH2 | 0.269 | 0.004 | 0.328 | −0.29 | 0.008 | −0.123 | −0.226 | 0.304 | 0.127 | 0.064 | −0.523 | −0.106 | 0.007 |
| SPG3A | 0.015 | 0.157 | −0.045 | −0.78 | −0.583 | 0.022 | −0.435 | 1.067 | 1.249 | 0.124 | 0.408 | −0.535 | 0.155 |
| SPPL3 | 0.553 | 0.407 | 0.499 | −0.051 | 0.383 | 0.004 | 0.509 | 0.493 | 0.421 | 0.907 | −0.315 | −0.428 | 0.233 |
| SQLE | −0.770 | 0.341 | −1.698 | 1.663 | 2.472 | 0.441 | 0.189 | −1.794 | −1.573 | 1.312 | −0.018 | 0.008 | 0.702 |
| SQRDL | 1.8285 | 1.1045 | 0.0015 | 0.2525 | −0.0415 | 0.3555 | −0.4335 | 1.1875 | 1.1585 | 1.0995 | 1.4075 | 0.8805 | 1.7295 |
| SREBF1 | −0.0765 | 0.3055 | −0.1505 | 0.0795 | −0.2905 | 0.2275 | 0.0725 | −0.1145 | −0.1585 | −0.4785 | −0.2665 | −0.1025 | −0.5045 |
| SRI | 1.9065 | 0.1635 | 0.5705 | −0.0265 | −0.4675 | −0.0165 | 0.0935 | −0.6685 | −0.6365 | 0.2435 | −0.4875 | 0.2225 | 0.4135 |
| SRPK1 | 0.1745 | −0.8215 | −0.7615 | −0.2885 | −0.5645 | −0.4085 | −0.0205 | 0.7465 | 0.7115 | 0.2305 | 0.1835 | −0.3485 | 0.1925 |
| SSFA2 | 0.5965 | 0.3795 | 0.2805 | −0.7195 | −0.1925 | −0.3025 | −0.6235 | 0.5285 | 0.4725 | 0.1635 | 0.3075 | 0.2916 | −0.0565 |
| SSR4 | 0.0735 | 0.2225 | −0.1745 | 0.3045 | 0.4405 | 0.0795 | −0.2545 | 0.2445 | 0.2955 | −0.0885 | −0.1885 | 1.0155 | 0.2465 |
| STARD3NI | −0.6885 | −0.4605 | −0.4795 | −0.4585 | −1.0935 | −0.0995 | −0.6695 | 2.1215 | 2.1185 | 0.8795 | 1.7976 | 0.7385 | 0.6265 |
| STARD7 | 3.1615 | 3.7965 | 2.9945 | 2.4525 | 2.5505 | 0.3715 | 1.3365 | −0.4385 | −0.4145 | 0.6595 | 2.4585 | 2.7025 | 1.0465 |
| STAT1 | −0.1115 | −0.3215 | −0.8035 | −0.3735 | −0.3215 | −0.0035 | −0.1765 | −0.2885 | −0.2955 | −0.0205 | −0.0095 | −0.1525 | 0.3395 |
| STC2 | −0.8045 | 1.2265 | −0.6415 | −0.1935 | −0.3355 | −0.3095 | −0.2495 | −0.2875 | −0.0635 | −1.0315 | −0.0685 | −0.0915 | −0.4035 |
| STK24 | −1.0235 | −1.0275 | −0.0165 | −0.7845 | −0.9915 | −0.2875 | −2.6505 | −0.1865 | −0.1525 | −0.8875 | −0.3945 | −1.2865 | −0.3015 |
| STK6 | 0.065 | −0.728 | −0.359 | 0.15 | −0.456 | 0 | 0.056 | 0.463 | 0.595 | −0.419 | 0.309 | 0.213 | −0.123 |
| STMN1 | 0.4405 | 0.0745 | 0.1225 | −0.2915 | −0.3595 | −0.2725 | −0.0225 | 0.7975 | 0.7775 | −0.2285 | 0.5955 | −0.0955 | 0.3565 |
| STX1A | 0.5735 | 0.3955 | −0.3355 | 0.1535 | −0.0235 | −0.2505 | −0.4565 | 0.3675 | 0.4145 | 0.6865 | −0.3805 | −0.0905 | −0.4465 |
| STX3A | 0.6495 | 0.2155 | −0.6575 | 0.1885 | 0.2735 | 0.1735 | 0.0375 | −0.3005 | −0.2905 | −0.0025 | 0.3225 | −0.6105 | −0.4305 |
| STYX | 1.2305 | 0.9005 | 0.4835 | 0.2745 | 0.6025 | 0.3435 | 0.2965 | 0.6335 | 0.6385 | 0.5305 | −0.6075 | −0.2855 | 0.6715 |
| SUGT1 | 0.32 | 0.1 | −0.331 | −0.184 | −0.106 | 0.033 | −0.591 | 0.713 | 0.742 | −0.194 | 0.424 | −0.299 | −0.323 |
| SUMF1 | −0.3395 | 0.9425 | −0.3935 | −0.5825 | −0.9745 | −0.4525 | −0.3495 | −0.3295 | −0.1915 | −0.2145 | −0.2485 | −0.6575 | −0.5895 |
| SUMO2 | 0.2405 | −0.0575 | −0.2675 | −0.5055 | −0.3215 | −0.4805 | −0.1545 | 0.2815 | 0.1075 | 0.3335 | 0.2395 | 0.2965 | −0.0175 |
| SUV39H2 | −0.6815 | 0.3585 | 0.6245 | −2.4505 | −0.5675 | −0.5085 | −0.2835 | 0.1015 | 0.0555 | −1.2685 | 1.4945 | −0.1575 | −0.2995 |
| SYAP1 | −0.1995 | 0.0775 | −0.3225 | −0.1725 | 0.2845 | −0.4665 | 0.4985 | −1.4575 | −1.3125 | −0.4645 | −0.8245 | −0.7295 | −0.2215 |
| SYCP2 | 1.482 | −0.193 | −1.335 | 1.63 | −0.283 | −0.554 | −0.027 | 1.389 | 1.353 | 0.35 | 1.372 | −0.9 | −0.401 |
| SYNGR1 | 0.1645 | 0.1205 | 0.0495 | −0.2275 | 0.3725 | 0.0175 | −0.0125 | 0.4505 | 0.3425 | 0.2365 | 0.5275 | −1.2505 | 0.6235 |
| SYTL2 | 0.1425 | −0.0135 | −0.1455 | 0.2025 | 0.1825 | 0.0645 | −0.0105 | −0.3545 | −0.2825 | −0.0945 | 0.3325 | −0.6455 | 0.1065 |
| TAF18 | −0.8445 | −0.3985 | 0.3325 | 0.1545 | 0.0655 | 0.0035 | 0.3745 | −0.0925 | 0.0105 | −0.4585 | 0.6705 | 0.3625 | −0.0115 |
| TAF8 | −0.118 | 0.155 | −0.349 | 0.248 | 0.007 | −0.077 | −0.296 | −0.098 | −0.072 | −0.35 | −0.373 | −0.704 | −0.589 |
| TARBP1 | 0.289 | 0.525 | −0.067 | −0.395 | −0.105 | 0.175 | 0.038 | 1.265 | 1.265 | 0.795 | 0.435 | −0.14 | 0.58 |
| TARS | 0.8545 | 0.0016 | 0.6435 | 0.3725 | 0.0855 | 0.3915 | 0.8665 | −0.0295 | 0.0316 | 0.7385 | −0.1715 | 0.1215 | 1.2185 |
| TATDN1 | 0.1316 | 0.1385 | 0.6135 | 0.1545 | 0.3885 | 0.2205 | −0.1325 | −0.0045 | 0.0416 | 0.0145 | −0.3745 | −0.2515 | 0.3265 |
| TBC1D12 | −0.7645 | 0.1525 | −0.3905 | 0.1685 | 0.2115 | 0.7095 | 0.2415 | −0.0575 | 0.0085 | −0.0085 | 0.3445 | 0.8355 | −0.0135 |
| TBC1D14 | 1.037 | −0.015 | 0.276 | 0.216 | 0.588 | 0.021 | 0.753 | 0.162 | 0.119 | 0.618 | 0.131 | 0.043 | 0.341 |
| TBC1D3 | −1.4815 | −0.3505 | 1.7375 | −0.3425 | 0.7005 | −0.4135 | 1.5555 | 2.5145 | 2.4215 | 0.7475 | 0.4385 | −1.0575 | 0.7445 |
| TBCA | 1.442 | 2.241 | 2.445 | 0.878 | 1.217 | 1.936 | 0.626 | 0.753 | 0.579 | 0.773 | 0.982 | −1.001 | 2.635 |
| TBX1 | 0.5575 | 0.0925 | −0.3085 | −0.1405 | −0.4216 | −0.2305 | −0.0075 | 1.1035 | 0.9095 | −0.4685 | 0.0945 | 0.2405 | 0.0445 |
| TBX3 | 1.89 | 0.96 | 0.384 | 1.375 | 0.583 | 0.093 | 3.127 | 0.588 | 0.535 | 1.853 | 3.115 | 0.751 | 1.256 |
| TCAP | 2.264 | 0.093 | 0.595 | 0.735 | 0.498 | 0.173 | 2.918 | 0.234 | 0.208 | 1.026 | 2.583 | 0.568 | 0.801 |
| TCEAL1 | 1.8235 | 0.4825 | −0.0205 | 1.9335 | 0.9725 | 0.3085 | 2.8105 | 0.3185 | 0.3235 | 1.3545 | 2.6965 | −0.0075 | 0.2685 |
| TCEAL3 | −0.99 | −0.436 | −0.82 | −0.34 | −0.11 | 0.49 | 0.684 | −1.366 | −1.206 | −0.291 | 0.528 | 0.491 | 0.78 |
| TCEAL4 | 0.31 | −0.559 | −0.238 | 0.269 | 0.253 | 0.231 | −0.811 | −0.881 | −0.78 | 0.044 | −0.195 | −0.504 | −0.221 |
| TCF15 | −0.002 | 0.088 | 1.058 | 0.101 | −0.227 | 0.142 | −0.119 | −0.87 | −0.921 | −0.879 | −1.371 | −0.455 | −0.522 |
| TCF3 | −0.113 | 0.156 | 0.089 | −0.169 | −0.06 | 0.2 | −0.134 | 1.066 | 1.088 | 0.35 | 0.814 | 0.023 | 0.076 |
| TCF7L2 | 0.3185 | 1.1035 | 0.3525 | −0.2915 | 0.1265 | 0.3105 | 0.3575 | 0.8975 | 1.0085 | −0.0215 | 0.1015 | 0.0235 | 0.4285 |
| TDRD7 | 1.1605 | 2.4645 | 1.0205 | 0.8835 | 1.4285 | 0.6745 | −2.7855 | −3.8555 | −4.0935 | 0.7235 | −3.3615 | 1.7025 | 0.7005 |
| TESK2 | 0.609 | 2.252 | 0.258 | −0.131 | −0.466 | 0.901 | 0.429 | 1.14 | 1.479 | 0.145 | 0.132 | −1.089 | 1.553 |
| TFAP2B | 0.047 | 0.278 | −0.048 | −0.966 | −0.819 | 0.093 | −0.853 | −0.544 | −0.595 | 0.336 | −0.005 | 0.005 | 0.766 |
| TFCP2L1 | 0.8085 | 1.9335 | 0.2585 | −0.1065 | 0.7435 | 1.6785 | 0.0215 | 2.1105 | 2.1795 | 1.0425 | 1.4775 | −0.4325 | 2.7935 |
| TFCP2L2 | 2.7755 | −0.3845 | 1.8825 | −0.9745 | 0.0855 | 1.2485 | −2.0025 | 2.3645 | 2.1365 | 2.1485 | 2.8905 | 0.9595 | 2.9505 |
| TFF1 | 0.6435 | −0.2535 | 0.0415 | 0.1365 | −0.2985 | 0.6845 | −1.3375 | −0.0715 | −0.0295 | 0.1575 | −0.4755 | −0.2795 | −1.2965 |
| TFF3 | −0.842 | 0.696 | −0.041 | 0.04 | −0.372 | 0.401 | −0.782 | 0.125 | −0.27 | 0.051 | −1.148 | 3.575 | 0.421 |
| TFRC | −0.0055 | 0.1695 | −0.3285 | −0.4265 | −0.3985 | −0.3435 | −1.2585 | 0.1115 | 0.1835 | 0.2085 | 0.1095 | −0.9185 | −0.5785 |
| TGFBR3 | 0.644 | 0.273 | 0.496 | −0.524 | −0.531 | −0.646 | −0.533 | 0.867 | 0.785 | −0.005 | −0.258 | 0.698 | 0.015 |
| THY28 | 0.8875 | 0.3045 | 0.8405 | −0.2695 | 0.4225 | 0.0245 | 0.1525 | 1.4245 | 1.3945 | 0.6035 | 0.8465 | 0.4915 | 0.1675 |
| TIF1 | −0.02 | 0.658 | 0.167 | −0.368 | −0.223 | −0.121 | 0.138 | 1.314 | 1.302 | 1.242 | 0.338 | 0.96 | 0.726 |
| TIGA1 | 0.063 | −0.737 | −0.58 | −0.775 | −0.648 | −0.299 | 0.073 | 0.238 | 0.298 | −0.373 | 0.263 | −0.862 | −0.168 |
| TIM14 | 0.2855 | 0.1855 | 0.8395 | 0.1875 | 0.1315 | 0.2175 | 0.3445 | −0.1395 | −0.0275 | 0.5685 | −0.4905 | −0.0895 | −0.1695 |
| TIMELES [symbol] | 0.8555 | 0.8285 | 0.9905 | −0.1665 | 0.0395 | 0.0825 | −0.3075 | −0.2515 | −0.1955 | 0.5765 | −0.5095 | 0.3285 | 0.2435 |
| TIMM9 | 0.292 | 0.5 | −0.044 | 0.405 | −0.387 | −0.273 | −0.411 | 2.234 | 2.495 | 0.464 | −1.342 | −1.511 | 0.01 |
| TINP1 | −0.5255 | −1.6995 | −0.8705 | −0.6005 | −0.8785 | −0.8845 | −2.8605 | −0.0385 | −0.0525 | −0.5655 | −0.8415 | −0.5255 | −0.4935 |
| [symbol] IP1208 | 0.523 | −0.611 | −0.592 | 0.307 | 0.096 | 0.656 | 0.901 | 0.021 | 0.008 | 0.646 | 0.764 | 0.253 | −0.059 |
| TK1 | 0.638 | −0.996 | −0.506 | −0.381 | 0.258 | 0.232 | 1.876 | 1.233 | 1.492 | 0.483 | −0.365 | 0.627 | 0.707 |
| TLE2 | −0.099 | 0.928 | 0.297 | −0.252 | −1.356 | −0.299 | 0.587 | 1.89 | 1.859 | 1.326 | 0.895 | 1.981 | −0.189 |
| TLR5 | 0.821 | 1.399 | 0.332 | −0.649 | −0.973 | −0.742 | 0.214 | 2.456 | 2.435 | 1.559 | 1.18 | 2.328 | 1.889 |
| TM4SF12 | 0.883 | 0.196 | 0.189 | 0.005 | −0.026 | −0.39 | 0.259 | 0.654 | 0.631 | 0.411 | 0.627 | 0.048 | −0.106 |
| TM4SF13 | 0.328 | 1.151 | 0.29 | 0.206 | 0.765 | −0.497 | 1.16 | 0.131 | 0.192 | 1.068 | −0.714 | −0.405 | −1.251 |
| TMEM14A | 0.4885 | 0.0505 | −0.0025 | 0.3315 | −0.1865 | 0.0475 | 0.3765 | 0.3785 | 0.4475 | 0.4995 | −0.3045 | 0.3055 | 0.4085 |
| TMEM25 | −0.7965 | 0.8415 | 0.3345 | −0.2755 | 0.4095 | −0.5115 | −0.3815 | −1.3085 | −0.7635 | −1.1535 | −0.5135 | 0.9775 | −0.4835 |
| TMEM4 | −0.277 | −0.169 | −0.709 | −0.372 | −0.294 | −0.54 | −1.399 | 0.26 | 0.215 | 0.191 | −0.31 | −1.606 | −1.209 |
| TMPRSS5 | −0.5 | −0.01 | −1.432 | −0.36 | −0.32 | 0.03 | −0.484 | −0.41 | −0.49 | −1.57 | 0.41 | −0.815 | −0.294 |
| TMSB10 | −2.951 | −0.482 | 0.496 | −0.041 | −1.312 | −0.201 | −0.803 | 0.363 | 0.392 | −1.708 | −0.545 | 1.228 | 0.835 |
| TMSNB | −1.0425 | −0.0555 | −0.6275 | −0.3765 | 0.1715 | 0.2475 | −1.1735 | −0.1715 | −0.1885 | −0.0115 | −0.8825 | −0.5205 | −0.3805 |
| TNFRSF1 [symbol] | −0.877 | −1.268 | −0.028 | −1.218 | −0.861 | 0.163 | −1.386 | −0.565 | −0.353 | −0.762 | −1.497 | −0.855 | −0.834 |
| TNFRSF2 [symbol] | 1.6145 | −0.4465 | −0.1715 | 1.8725 | 0.8755 | 0.5545 | 0.1635 | 1.0985 | 0.9705 | 0.9275 | 1.2565 | −1.2475 | 0.0565 |
| TNFRSF2 [symbol] | −0.9095 | 0.4915 | −0.3995 | 0.3885 | −0.8625 | 0.5255 | −0.4225 | −0.5925 | −0.2685 | −0.7995 | −0.1125 | 0.3885 | 0.0985 |
| TNFSF4 | −0.4916 | −0.5795 | −0.1845 | −0.0405 | −1.0165 | −0.0355 | −0.1565 | 0.0385 | 0.0585 | 0.0585 | −0.7115 | −0.3515 | −0.7225 |
| TNFSF7 | −0.6995 | −0.0025 | −0.5295 | −0.5125 | −0.5295 | −0.4735 | −0.8835 | −0.5235 | −0.2805 | −0.7045 | −1.1625 | −0.7425 | −0.5745 |
| TNIK | −0.8885 | −0.6855 | −0.4446 | −0.2795 | 0.2725 | −0.0705 | −0.8845 | −0.5315 | 0.0245 | −1.1585 | −0.7215 | −0.2905 | 0.5495 |
| TNNC2 | 3.125 | 0.598 | −0.27 | 0.583 | 0.546 | −0.1 | −0.261 | 4.605 | 4.631 | 1.145 | −0.395 | −0.769 | 1.803 |
| TNNI2 | 0.334 | 1.308 | 0.402 | 0.002 | 0.685 | 0.019 | 0.79 | 1.352 | 1.12 | 0.951 | −0.199 | 0.624 | −0.138 |
| TNNI3 | 1.0885 | −0.0065 | 0.7265 | 0.2425 | −0.3535 | 0.1645 | 0.2305 | 1.1835 | 1.2195 | 2.0315 | 1.4245 | 0.5945 | 0.5195 |
| TOB1 | 0.1285 | 0.2425 | 0.0435 | 0.4645 | 0.0976 | 0.1915 | 0.2475 | 1.2715 | 1.3515 | 0.7195 | −0.3685 | −0.5745 | 0.2985 |
| TOM1L1 | −0.0275 | −0.2525 | −0.7785 | −0.8375 | −0.0495 | 0.2925 | −0.9915 | −0.8185 | −0.5385 | −0.7165 | 0.2045 | 0.8155 | −0.6255 |
| TOMM7 | −0.2845 | −2.1325 | −0.5385 | −0.8805 | −0.6715 | −0.5105 | −2.4055 | 0.2385 | 0.2045 | −0.7875 | 0.5885 | 0.1995 | 0.0205 |
| TOP1MT | 0.183 | −0.026 | 0.952 | 0.58 | 0.34 | 0.368 | 0.104 | −0.302 | −0.355 | −0.187 | 0.196 | 0.095 | 0.138 |
| TOP2A | −0.0105 | 0.3635 | −0.0455 | −0.3895 | −0.2965 | 0.3715 | −0.1955 | 0.9385 | 0.5305 | 0.0145 | −0.3095 | −0.1375 | −0.3275 |
| TOP2B | −0.987 | −0.056 | −0.637 | −1.448 | −0.594 | −1.154 | −1.51 | 0.605 | 0.551 | −0.822 | 0.133 | −0.182 | −0.159 |
| TOPBP1 | 0.8115 | 0.4075 | 0.5785 | −0.3105 | −0.0495 | 0.2095 | 0.0975 | 1.0915 | 1.0465 | 0.5965 | 0.1515 | 2.0485 | −0.2245 |
| TOPK | −0.2155 | −0.4365 | 0.3145 | 0.0675 | 0.2925 | 0.4215 | −0.0485 | 0.5005 | 0.3955 | 0.0825 | −0.3045 | −0.1755 | −0.8415 |
| TP53AP1 | 0.6975 | 1.8275 | 0.3935 | 0.0115 | 1.2115 | 1.1845 | −0.5965 | 0.2735 | 0.1375 | 1.1845 | −0.8645 | 1.8255 | 1.3655 |
| TP53BP2 | −3.1905 | −0.0825 | −0.8385 | −2.2245 | −1.5725 | −0.2185 | −2.0385 | −1.0845 | −0.9235 | 0.2495 | −1.4005 | −1.8785 | −0.6355 |
| TP53INP1 | −0.5175 | −0.6625 | 0.0335 | −0.4355 | −0.5575 | −0.0225 | −0.6495 | −0.0115 | 0.1935 | −0.2555 | −0.1345 | −0.9455 | −1.0055 |
| TPD52L1 | −0.992 | −0.878 | −1.047 | −0.063 | −0.867 | 0 | −0.939 | 1.734 | 1.652 | 0.468 | −0.766 | 1.457 | 0.817 |
| TPRT | 0.0665 | −0.4435 | −0.0015 | −0.8585 | −0.4196 | 0.0015 | −0.2665 | −1.0975 | −1.0565 | −0.6275 | −0.0865 | 0.0135 | 0.0055 |
| TRA@ | 0.975 | 0.17 | 0.278 | 0.511 | 0.133 | −0.475 | 0.908 | −0.081 | −0.142 | 0.618 | −0.151 | −1.035 | −0.52 |
| TRAF4 | 0.288 | 0.485 | −0.503 | −0.17 | −0.344 | 2.881 | 0.257 | 0.765 | 0.788 | 1.36 | 4.449 | 0.199 | 5.849 |
| TRAPPC4 | 0.589 | 1.934 | 0.153 | −0.507 | 1.015 | 0.291 | 1.499 | 1.274 | 1.724 | −0.084 | −0.413 | −0.667 | −0.269 |
| TRH | −0.2745 | −0.3665 | −0.1476 | −0.1425 | −0.8905 | −0.3005 | 0.5535 | 0.3065 | 0.2245 | −1.8745 | −0.8665 | −1.4935 | −0.7525 |
| TRIM17 | 0.3475 | −0.2815 | 0.9465 | 0.7475 | 0.7035 | 0.3975 | −0.0485 | 0.9605 | 0.7545 | 0.8645 | 0.1815 | −0.5815 | −0.4745 |
| TRIM2 | 0.7585 | 0.8105 | −0.0005 | 0.0685 | −0.0435 | −0.0065 | 0.1065 | 0.4285 | 0.5115 | 0.7245 | 0.0765 | 0.2725 | 0.3805 |
| TRIM6 | −0.048 | 0.3 | 0.077 | 0.313 | 0.939 | 0.901 | 0.049 | 0.519 | 0.468 | 0.308 | 0.01 | 0.85 | 1.336 |
| TRIP3 | −0.3855 | 0.1795 | 0.2105 | −0.6225 | −0.0485 | −0.3055 | 0.9505 | 0.4195 | 0.3405 | 0.1225 | 0.1025 | 0.4685 | 0.8245 |
| TRPS1 | 0.7365 | 0.5855 | −0.3105 | 0.6555 | 1.2195 | 0.3355 | 0.5835 | 0.1145 | 0.0955 | 0.6565 | 0.2085 | 0.7085 | 0.5265 |
| TSG101 | −0.0095 | 0.3905 | 0.0485 | 0.4585 | 0.1705 | −1.0385 | −0.5875 | −0.5215 | −0.3415 | 0.5205 | −0.2585 | 0.0605 | 0.2775 |
| TTC10 | −1.1305 | 0.0035 | −0.6855 | −0.8945 | −0.4705 | −0.4175 | −1.6845 | 0.0785 | 0.1555 | −0.3445 | −0.6455 | 0.4925 | −0.2435 |
| TTC12 | −1.03 | −0.185 | −0.277 | −0.064 | −0.421 | −0.243 | −0.519 | −0.35 | −0.377 | −0.399 | −0.279 | −0.194 | −0.477 |
| TTK | −0.3965 | −0.6845 | −0.2875 | −0.5765 | −0.2715 | −0.4945 | −0.3325 | −1.3435 | −0.7435 | −0.4765 | −0.2995 | 0.1025 | −0.3495 |
| TTLL4 | 0.766 | −0.831 | −0.038 | 0.384 | −0.047 | 0.473 | −0.568 | −0.316 | −0.122 | −1.175 | 1.226 | −0.748 | −1.105 |
| TTYH1 | −0.1885 | −0.6705 | −0.4025 | 0.4905 | 0.1185 | 0.2885 | 0.7075 | −0.7355 | −0.5925 | −0.7105 | −0.0675 | 0.2045 | −1.0575 |
| TUBB3 | −0.3845 | 0.4455 | 0.1305 | 0.1495 | −0.0315 | 0.2155 | −0.5335 | 0.3155 | 0.3815 | 0.5815 | 0.5085 | −0.9625 | 0.1185 |
| TULP3 | 0.005 | −0.266 | −0.725 | −0.863 | −1.402 | −0.51 | −2.255 | −0.708 | −0.684 | 0.158 | −0.191 | −0.195 | −0.335 |
| TXN | 0.309 | −0.12 | −0.028 | −0.296 | −0.054 | 0.129 | 0.092 | 0.469 | 0.457 | −0.568 | 0.098 | −0.949 | −0.095 |
| TYMS | 0.6375 | 8.5725 | 0.2115 | 0.4155 | −0.7085 | 0.2155 | −1.6615 | −0.1645 | −0.1776 | −1.2285 | −0.3385 | −2.5945 | −0.7075 |
| U5-116KD | 0.4915 | 1.2025 | −0.1325 | 0.1145 | −0.0515 | 0.1325 | 0.2865 | 0.7835 | 0.7605 | 0.1925 | 0.0485 | −0.1055 | 0.1175 |
| UBE2E3 | 0.142 | −0.406 | −1.052 | 0.159 | 0.388 | −0.015 | −0.405 | 0.323 | 0.224 | 0.197 | 0.132 | 1.034 | 0.321 |
| UBL5 | 0.5705 | 0.6585 | −0.5305 | 0.9975 | 1.0135 | 0.5435 | −0.7605 | −0.1325 | −0.2525 | 0.0365 | 0.8125 | −1.6835 | −1.3105 |
| UBN1 | 0.425 | −0.151 | −0.153 | 0.017 | 0.248 | −0.02 | −0.961 | −0.702 | −0.606 | 0.376 | −0.015 | −0.138 | −0.007 |
| UCHL1 | 1.27 | 1.018 | 0.867 | 0.519 | 1.341 | 0.592 | 1.101 | 1.457 | 1.448 | 1.01 | −0.739 | 0.58 | 2.027 |
| UCHL3 | 0.562 | −0.344 | −0.525 | −0.625 | −0.532 | −0.294 | −0.402 | −0.177 | −0.18 | −0.342 | −0.621 | −0.584 | −0.704 |
| UGCG | −0.0295 | −0.4305 | 0.2425 | −0.2785 | −0.6035 | 3.5945 | −0.2035 | −1.5755 | −1.3385 | −0.7925 | −0.6815 | 1.5565 | −1.0715 |
| UGCGL1 | 0.6735 | −0.3965 | −0.5955 | 0.1745 | 0.2075 | −0.1125 | −0.0275 | 0.1395 | 0.1755 | −0.7065 | −1.3285 | −0.1175 | −0.6735 |
| UGT2B7 | 0.5935 | 0.3025 | −0.5275 | −0.9155 | −0.2285 | −0.3285 | 0.3425 | 0.6755 | 0.7085 | 0.3365 | 0.1815 | −0.1875 | 0.8385 |
| UNC13D | 0.4 | 0.549 | 0.551 | 0.114 | 0.462 | 0.152 | −0.395 | 0.527 | 0.619 | 0.661 | 0.568 | −0.392 | 0.4 |
| VAMP8 | −0.217 | −0.139 | −0.472 | 0.127 | 0.12 | 0.041 | −0.888 | 0.5 | 0.327 | 0.588 | 0.019 | 0.308 | −0.409 |
| VAPA | −0.0155 | 0.6995 | 1.1155 | 0.9675 | 1.4985 | 0.5745 | 0.9385 | 1.6715 | 1.5865 | 1.5795 | 0.2965 | 0.7475 | 1.1295 |
| VAV1 | 0.033 | 1.563 | 0.129 | 0.126 | 0.923 | 0.175 | −1.087 | −0.988 | −1.037 | −0.699 | −0.069 | 0.576 | −0.998 |
| VAV3 | 0.019 | −0.451 | 0.382 | −0.226 | −0.119 | −0.364 | 0.295 | 0.232 | 0.191 | 0.165 | −0.133 | −0.065 | −0.142 |
| VCX3 | −0.048 | −0.018 | −0.291 | 0.103 | −0.222 | 0.146 | −0.051 | 0.133 | 0.234 | 1.442 | −0.22 | −0.358 | 0.082 |
| VDAC2 | −0.1615 | 0.4415 | 0.0985 | −0.6516 | 0.1725 | −0.1585 | −0.1716 | −0.1955 | −0.5155 | −0.5805 | −0.3815 | 0.4885 | −0.2715 |
| VEST1 | −0.578 | −0.716 | 0.164 | −0.246 | 0.059 | 0.155 | −0.105 | −0.449 | −0.409 | −0.014 | −0.018 | −0.664 | −0.247 |
| VGLL1 | −0.1525 | 0.2545 | −0.2875 | −0.4845 | −0.3875 | 0.1405 | −0.2645 | 0.0465 | 0.0595 | −0.0735 | −0.3845 | 0.0685 | 0.0335 |
| VMD2L2 | −0.389 | −0.985 | −0.879 | −0.051 | 0.837 | 0.003 | −0.218 | −0.205 | −0.488 | 0.101 | −0.551 | 0.024 | −0.546 |
| VSX1 | −0.124 | −0.726 | 0.649 | −0.305 | 0.133 | 0.21 | 0.025 | 0.293 | 0.258 | 0.081 | −0.304 | −0.231 | −0.05 |
| WARP | −0.186 | −0.092 | −0.757 | 0.296 | 0.785 | 0.301 | 0.806 | −0.87 | −0.79 | −0.468 | −0.012 | −0.062 | 0.22 |
| WDR26 | 1.6855 | 0.2045 | −0.1455 | 0.2945 | 0.6905 | −0.3275 | −0.3125 | 0.0025 | 0.0545 | −0.4925 | 2.2485 | −0.7475 | 2.3135 |
| WDR6 | 1.27 | −0.277 | −0.292 | 1.05 | 0.351 | 0.21 | 2.018 | 0.365 | 0.403 | 0.051 | 0.88 | 0.668 | 0.853 |
| WHSC1L1 | −0.659 | 0.151 | −0.120 | −0.489 | −0.48 | −0.275 | −0.189 | 0.608 | 0.506 | −0.74 | 0.091 | −0.169 | 0.041 |
| WNT3 | 0.2515 | −0.1285 | −0.1135 | 0.3055 | −0.8825 | −0.0315 | −0.5055 | −0.0135 | 0.1625 | −0.3115 | −0.1945 | 0.0235 | −0.5635 |
| WNT6 | 0.8125 | −0.6715 | 0.6215 | 0.3065 | 0.8085 | 1.5315 | 1.2005 | 1.1805 | 1.0335 | −0.2375 | 0.0355 | 1.9275 | 1.4295 |
| WSB2 | −0.2335 | −0.7035 | −0.4695 | −0.2485 | −0.1185 | −0.1835 | 0.5575 | −0.6715 | −0.9145 | −0.6405 | 0.2155 | 0.3055 | −0.1375 |
| WWP1 | −0.0765 | 0.5435 | 1.2945 | 0.5345 | 1.5555 | 1.1765 | 1.2425 | 0.1005 | 0.4505 | 0.3935 | 0.4435 | 1.2195 | 1.5065 |
| WWP2 | 0.392 | −0.116 | 0.405 | 0.217 | 0.146 | 0.26 | 0.087 | −0.008 | 0.019 | 0.879 | −0.373 | 0.402 | 0.035 |
| XBP1 | −0.518 | −0.172 | −0.879 | −0.327 | −0.028 | −0.645 | −0.099 | −0.979 | −0.87 | −1.154 | −0.544 | 0.606 | −0.01 |
| XYLT2 | 0.1305 | −0.1435 | −0.2775 | 0.2635 | −0.1315 | −0.1325 | 0.0365 | −0.1585 | −0.0715 | −0.4275 | −0.1265 | −0.3035 | −0.5965 |
| YBX2 | 0.526 | 1.008 | 0.64 | −0.407 | −0.319 | −0.122 | 0.231 | 0.409 | 0.487 | 1.074 | 1.453 | 0.386 | 0.757 |
| YIF1 | 0.1555 | 0.7645 | 1.1385 | −0.1345 | 0.5115 | −0.0645 | 1.2975 | −0.7575 | −0.8465 | 0.5255 | −0.0685 | 0.7285 | 0.7245 |
| ZA20D3 | −0.0005 | −0.1735 | 0.4755 | −0.0845 | 0.7515 | −0.0055 | −0.8505 | −1.2075 | −1.2835 | −0.1775 | −0.7365 | −0.2815 | −0.8615 |
| ZAP128 | 0.0145 | 0.6805 | 0.1925 | 0.1645 | 0.5335 | −0.0505 | 0.6215 | 0.5025 | 0.4075 | 0.2255 | −0.4545 | −0.1505 | 0.9055 |
| ZBED3 | −0.496 | 0.102 | 0.638 | −0.331 | 0.097 | 0.003 | 0.018 | −0.08 | −0.161 | −0.41 | −0.003 | 0.358 | 0.019 |
| ZBTB4 | 0.23 | −0.273 | −0.185 | 0.039 | −0.082 | 0.457 | −0.221 | −0.596 | −0.439 | −0.024 | −0.041 | −0.231 | −0.202 |
| ZCWCC1 | −0.7685 | 0.1385 | −0.9905 | −0.5085 | −0.6845 | −0.0245 | 0.6025 | 0.5105 | 0.5485 | −0.1415 | 0.2825 | −0.5795 | 0.0295 |
| ZCWCC2 | −0.357 | −0.16 | 0.528 | 0.774 | 0.390 | 0.277 | 0.306 | −1.065 | −0.986 | −0.704 | −0.889 | 0.147 | 0.087 |
| ZDHHC13 | 1.0275 | 0.5285 | 0.8825 | 1.3285 | 0.7135 | 0.8375 | −0.0225 | 0.2505 | 0.3835 | −0.1105 | −0.4855 | 0.1865 | −1.0495 |
| ZDHHC14 | 0.1245 | 0.3055 | −0.0055 | 0.0845 | −0.0675 | −0.3215 | −0.0685 | 0.0865 | 0.1255 | 0.4675 | −1.0115 | −0.3075 | 0.1035 |
| ZFHX4 | 0.0015 | 0.3355 | 0.5485 | 0.0275 | 0.2405 | −0.0355 | 0.0355 | −0.4725 | −0.4585 | −0.3465 | −0.6395 | −0.3435 | −0.0565 |
| ZFYVE21 | 0.611 | 0.548 | 0.078 | −0.024 | 0.535 | 0.22 | 1.523 | 1.033 | 1.102 | 0.395 | 0.215 | 0.005 | 1.312 |
| ZNF133 | 0.396 | 0.357 | 0.75 | −0.232 | 0.261 | −0.175 | 0.014 | 0.471 | 0.527 | 0.248 | 0.213 | 0.276 | 0.479 |
| ZNF165 | −0.1115 | 0.1485 | −0.0145 | 0.1095 | 0.4305 | 0.1055 | −0.1895 | 0.3545 | 0.2925 | 0.0045 | 0.0725 | −0.0545 | −0.0195 |
| ZNF24 | −0.204 | −0.234 | −0.06 | 0.166 | 0.264 | 0.09 | 0.444 | −0.097 | −0.182 | 0.441 | 0.761 | −0.355 | −1.174 |
| ZNF318 | −0.0595 | 0.0845 | −0.0195 | 0.0875 | −0.0315 | 0.0605 | 0.0075 | −0.2455 | −0.4195 | 0.0475 | 0.2035 | 0.1165 | 0.2775 |
| ZNF54 | −0.7415 | −0.9325 | −0.7005 | 0.3745 | −0.7615 | 0.5795 | −1.2955 | −0.9395 | −0.8985 | −0.8755 | −0.8425 | −1.0515 | −1.6985 |
| ZNF432 | −0.1505 | 0.5285 | −0.3945 | 0.2585 | −0.1895 | 0.0935 | −0.0425 | −0.7585 | −0.8325 | −0.2755 | −0.1565 | −0.2495 | 0.0125 |
| ZNF482 | 0.15 | −0.808 | 1.592 | −0.296 | 1.002 | 1.037 | 2.287 | −1.068 | −1.085 | 0.325 | 0.293 | 1.582 | 1.146 |
| ZNF505 | 0.715 | 0.085 | −0.304 | 0.464 | 0.617 | −0.185 | 0.529 | −1.037 | −1.079 | 0.585 | −0.584 | 0.303 | −0.029 |
| ZNF552 | 1.1225 | 0.3065 | 1.2705 | −0.0585 | 0.0175 | 0.4055 | 2.3465 | −0.6075 | −0.4555 | −0.2085 | 0.8545 | 1.2795 | 0.8335 |
| ZNF577 | −0.2505 | −0.0545 | −0.3945 | −0.1095 | 0.1145 | 0.0105 | 0.1025 | −0.8035 | −0.7115 | −0.3545 | −0.2575 | −0.3355 | 0.3165 |
| ZNF587 | 0.7405 | −0.3455 | 0.3895 | 0.0825 | 0.2795 | −0.2115 | 0.0085 | 0.9405 | 0.9745 | 0.3935 | 0.2285 | 0.0485 | 0.5955 |
| ZNF598 | 0.3175 | 0.4595 | 0.2905 | −0.0235 | 0.4855 | −0.0985 | −0.4675 | 0.0085 | −0.0845 | 0.4755 | 0.3725 | 0.3305 | 0.4645 |
| ZNF599 | 0.432 | 0.495 | 0.482 | −0.185 | 0.087 | −0.136 | −0.117 | −0.971 | −1.537 | 0.048 | −0.288 | −0.461 | −0.161 |
| ZNF606 | 0.168 | 0.234 | 0.529 | −0.343 | −0.2 | −0.129 | 0.448 | 0.517 | 0.650 | −0.04 | 0.213 | −0.857 | 0.48 |
| ZNF614 | 0.14 | 0.289 | −0.152 | −0.341 | 0.232 | 0.433 | −0.483 | −0.134 | −0.078 | −0.121 | 0.455 | 0.278 | 0.382 |
| ZNF639 | | | | | | | | | | | | | |
| ZNF7 | | | | | | | | | | | | | |

| | Sample | | | | | | | | |
|---|---|---|---|---|---|---|---|---|---|
| | 9830-0203 | PB249 | UB-120 | BC00078 | BC00017 | BR-02-704 | BR-99-062 | PB293 | PB407 |
| | Class | | | | | | | | |
| Symbol | LUMINAL | LUMINAL | LUMINAL | LUMINAL | LUMINAL | LUMINAL | LUMINAL | LUMINAL | LUMINAL |
| | −0.3365 | 1.1175 | −1.6365 | 2.8155 | 0.2005 | 1.6365 | 0.2715 | 2.4685 | 1.8095 |
| | 1.573 | 1.06 | 0.407 | −0.271 | 0.058 | −0.122 | −0.212 | 0.311 | 0.082 |
| ABAT | −0.187 | 1.552 | −1.02 | 1.578 | 0.168 | −0.944 | −0.457 | −2.235 | −3.138 |
| ABC1 | 0.8505 | 0.8335 | −0.5525 | 0.2735 | 0.2685 | −1.2775 | −1.6525 | −1.3225 | 1.8565 |
| ABCC11 | −0.2815 | 1.0585 | 0.3825 | −0.1665 | −0.6415 | −3.1065 | 0.3795 | −0.8305 | 0.0955 |
| ABCC3 | −0.873 | −1.158 | 0.042 | 0.218 | −0.289 | 0.458 | 0.309 | 0.274 | −0.224 |
| ACAS2L | −0.1535 | 0.0965 | −0.0485 | 0.1505 | 0.0315 | 0.3975 | −0.1035 | 0.5915 | −0.4035 |
| ACAT1 | −0.958 | 0.662 | −0.888 | 2.906 | 2.483 | 0.541 | 1.205 | 1.894 | 1.949 |
| ACAT2 | 0.4965 | 1.4675 | 1.1315 | 0.4715 | −0.2475 | 0.1415 | −0.0535 | −0.3715 | −0.4545 |
| ACOX2 | 0.8955 | 0.1065 | −0.9195 | 0.7535 | −0.2005 | −0.1165 | 1.2685 | 0.0205 | 0.1135 |
| ACDX3 | 0.4595 | 0.7325 | 1.7415 | −0.3815 | −0.5225 | 2.4865 | −0.8255 | −0.8505 | −0.0845 |
| ACSL3 | −1.024 | −0.425 | −0.199 | −0.161 | −0.735 | −0.622 | −0.846 | −0.565 | 0.243 |
| ACTA1 | −0.0125 | 0.4165 | −0.1485 | −0.1345 | 0.0025 | −0.1085 | 0.0135 | 0.0325 | 0.3845 |
| ACTB | −0.5895 | −0.1415 | 1.5575 | −1.2895 | −0.8215 | −0.4205 | 1.7345 | 0.0875 | 0.9435 |
| ADAM33 | 0.022 | 0.107 | −0.455 | 0.094 | 2.992 | 1.183 | −0.798 | 2.196 | −0.158 |
| ADAMDE [symbol] | 0.5875 | −1.1645 | −0.4885 | −0.4355 | −0.3495 | 1.7085 | 0.0055 | −1.8635 | 0.2925 |
| ADPN | 0.7305 | 0.4755 | −1.3485 | 1.1375 | −0.7745 | −0.9995 | 3.3425 | −0.7435 | 2.7785 |
| ADPRHL1 | 3.0895 | 2.0585 | 1.7335 | −0.4195 | 3.2345 | 1.5945 | 1.7726 | 1.8925 | 0.3045 |
| ADRA2C | −0.3535 | −0.2215 | 0.1255 | 0.6265 | 1.1375 | 3.3695 | −0.5785 | 1.0515 | 1.1975 |
| AGR2 | 2.8255 | 1.8775 | −0.1815 | 3.3545 | 1.8695 | −0.4055 | 3.6055 | 0.0675 | 0.1255 |
| AGT | 0.175 | 0.592 | 0.037 | 0.333 | 0.4 | −0.688 | 0.218 | 0.401 | 0.502 |
| AGTR1 | 0.271 | 0.39 | 3.607 | −0.516 | −0.925 | −1.12 | 0.937 | 1.226 | −0.152 |
| AHCYL1 | −1.954 | −0.075 | −0.509 | −0.069 | 0.024 | 0.95 | −0.869 | −0.031 | −0.304 |
| AHNAK | −2.038 | −1.877 | 0.227 | −1.192 | −0.212 | −0.736 | −2.212 | −0.816 | −1.581 |
| AHR | −0.0965 | −0.2945 | 0.4785 | 0.3265 | −0.0965 | −0.1745 | −0.8055 | −0.2415 | −0.6895 |
| AIM1 | 0.4255 | −0.9945 | 0.1755 | −0.0165 | 0.2085 | −0.2515 | 0.4605 | −0.8605 | −1.0075 |
| AK2 | −1.3475 | −0.3395 | −0.8265 | 2.3335 | −0.8815 | −0.7825 | −1.6615 | −0.9015 | −0.5525 |
| AK3L1 | 0.534 | −0.803 | 1.111 | 0.108 | 0.07 | 0.464 | −0.265 | 1.063 | −0.233 |
| AK5 | 0.002 | 0.187 | −0.101 | −0.19 | 0.139 | −0.757 | −1.31 | 0.18 | 0.008 |
| AKAP1 | 0.5075 | 0.3935 | 2.0035 | 0.1045 | 0.4255 | −0.4675 | −0.2475 | 2.0895 | 0.1875 |
| AKR1A1 | −1.145 | −0.707 | −0.549 | −0.248 | −0.301 | 0.625 | 1.297 | 0.175 | 0.324 |
| AKR7A3 | −0.5065 | 0.1335 | −0.2935 | −0.6395 | 0.4035 | 1.5295 | 1.0705 | 3.4775 | −0.5645 |
| ALG5 | 2.2465 | 1.5665 | −2.2775 | −2.4285 | 0.3925 | −0.9365 | −1.9165 | 1.4335 | 1.5605 |
| ALG8 | −0.5095 | 0.5245 | −0.3075 | −0.1935 | 0.0965 | −0.9075 | −0.3375 | −0.3505 | 0.4235 |
| AMFR | 1.072 | 0.71 | 1.35 | 0.564 | 0.367 | 0.94 | 0.373 | 0.521 | 0.204 |
| AMPD2 | −1.238 | 0.268 | 2.879 | 0.152 | 0.684 | 0.943 | 0.627 | −0.404 | 0.062 |
| ANAPC11 | −0.582 | −0.331 | 0.272 | −0.112 | −0.05 | −0.351 | 0.467 | 0.292 | −0.006 |
| ANKRD22 | −0.661 | 0.248 | −0.023 | −0.483 | 1.058 | 1.563 | −0.03 | 0.047 | 0.655 |
| ANKRD27 | 0.078 | −0.038 | −0.139 | −0.371 | 0.392 | 0.305 | 0.828 | 0.201 | −0.099 |
| ANLN | −1.27 | −1.623 | −3.989 | −2.758 | −0.545 | −1.87 | −1.56 | −3.155 | −2.679 |
| ANP32A | 0.168 | 0.281 | 1.033 | 1.153 | 0.626 | 1.57 | 0.542 | 1.114 | 0.07 |
| ANXA3 | −2.0185 | 0.9065 | 0.7165 | −0.0295 | 1.9225 | −0.6155 | 1.8125 | 0.7245 | 0.3375 |
| APBB2 | −0.5285 | −0.9405 | −1.4755 | −0.5665 | −1.1995 | −0.9235 | −1.2835 | −0.8255 | −1.0735 |
| APOBEC3 | 0.648 | −1.318 | 0.417 | 0.858 | 0.88 | −0.078 | −0.981 | 0.351 | −1.127 |
| AQP5 | 4.521 | 4.174 | −1.014 | −0.457 | 2.635 | −2.721 | 0.316 | −1.201 | 3.46 |
| AR | −0.0425 | 0.0395 | 0.3885 | 0.5175 | −0.2395 | 0.4525 | 0.3485 | −0.1755 | 0.1205 |
| AREG | −0.933 | −1.774 | 0.235 | 0.808 | −0.306 | −1.299 | −0.824 | 0.378 | −0.867 |
| ARF1 | 0.1715 | 0.1975 | 0.8885 | 0.7425 | 0.9785 | 2.2385 | 1.2725 | 0.7575 | 0.8015 |
| ARHGDI [symbol] | −0.458 | −0.015 | −0.277 | 0.001 | 0.328 | −0.104 | 0.505 | −0.222 | −0.196 |
| ARL6IP | 0.9125 | 1.0325 | −0.7085 | 0.9325 | 1.1105 | 1.0105 | 0.3275 | 0.7155 | 0.3955 |
| ARL6IP2 | 0.891 | 0.14 | −1.082 | 1.883 | 2.007 | 0.821 | 1.541 | 0.184 | 0.054 |
| ARMCX2 | 0.825 | 0.394 | −1.084 | 1.799 | 2.026 | 0.672 | 1.343 | 0.122 | 0.449 |
| ARNT2 | −0.32 | −0.182 | −0.157 | 0.959 | 0.189 | 0.488 | 0.458 | 0.13 | 0.703 |
| ARNT2 | −0.7275 | −0.3175 | −0.2405 | −0.5225 | −0.3875 | −1.1865 | −1.8145 | −0.8725 | −0.4475 |
| ARP3BET [symbol] | 0.2295 | 0.4675 | −0.0245 | 0.0755 | 0.0935 | −0.0085 | 0.1435 | −0.2235 | 0.1885 |
| ARPC1B | −0.704 | −0.518 | −0.204 | 0.179 | 0.167 | 2.915 | 1.614 | −0.97 | −0.359 |
| ARPC5L | −0.8965 | −1.4695 | −0.2195 | 0.0955 | −0.3635 | 0.6245 | −0.5075 | −0.9795 | −1.1585 |
| AS89 | 0.16 | −0.158 | 1.265 | −0.718 | −0.383 | 0.468 | 2.594 | 2.645 | 2.711 |
| ASF1A | −0.7255 | −0.6705 | −0.0585 | −0.5875 | 0.7995 | 1.0225 | 0.6725 | 0.0575 | 0.2825 |
| ASH2L | −0.306 | 0.445 | 0.272 | 0.147 | 1.684 | 1.352 | 0.488 | 0.319 | 0.542 |
| ASK | −0.473 | −0.598 | −1.849 | −1.118 | −0.411 | −1.556 | −1.101 | −1.311 | 0.787 |
| ASPM | −0.1055 | 1.2725 | 2.4675 | −0.2785 | 1.4885 | 1.6645 | 1.2245 | 0.5215 | 0.5545 |
| ASS | −0.3905 | 0.1255 | 0.4685 | −0.0405 | 0.0705 | −0.4895 | −0.9145 | 0.7225 | −0.2995 |
| ATAD2 | 0.1795 | 0.8155 | −0.2025 | 0.6025 | 0.9255 | 1.1595 | −1.9945 | −0.7005 | −1.3455 |
| ATF5 | −0.231 | −0.22 | 0.612 | 0.438 | 0.52 | 0.361 | 0.122 | −0.003 | −0.475 |
| ATF7IP2 | 0.15 | −1.093 | 0.787 | −0.261 | −0.045 | 0.773 | 0.071 | 0.152 | 0.008 |
| ATP2B1 | −0.2 | −0.389 | −0.46 | −0.294 | 0.197 | 0.965 | 0.285 | 0.888 | −0.079 |
| ATP5G1 | 0.1685 | 1.0875 | 0.5425 | 0.1455 | −0.0025 | 0.4265 | 0.5945 | 0.4945 | 0.8955 |
| ATP5O | −1.388 | 0.04 | −0.57 | 1.144 | −0.994 | −1.702 | −1.844 | −0.882 | −0.657 |
| ATP6AP1 | −1.024 | −0.82 | 0.168 | −0.455 | −0.012 | 0.068 | 1.299 | −0.657 | −0.681 |
| ATP6V0A4 | 0.988 | 0.926 | 0.131 | 0.338 | 0.029 | −0.406 | 0.964 | −0.648 | 0.251 |
| ATP6V1B [symbol] | −0.1395 | 0.6645 | 0.8395 | 0.6435 | 0.8255 | 0.3635 | 0.3515 | 1.0095 | 1.3505 |
| ATP6V1G [symbol] | −0.536 | −0.544 | −0.212 | 0.768 | 0.605 | 0.584 | 1.316 | 0.379 | −0.137 |
| ATPIF1 | −0.7245 | −1.1975 | −0.9595 | −0.4285 | −0.0055 | −0.6525 | −1.5876 | −0.7895 | −0.3625 |
| ATXN10 | 0.724 | −0.002 | −1.012 | −0.624 | −0.303 | −2.13 | −2.29 | −1.132 | −0.377 |
| B3GNT5 | 0.7615 | 0.0815 | −0.1015 | −0.1315 | −0.6005 | −0.0555 | 0.8895 | 0.7665 | −0.2785 |
| BACE2 | 0.3285 | 0.3125 | 0.3975 | −0.1435 | 0.0245 | 0.2905 | 1.7705 | 1.5025 | 1.3885 |
| BAG1 | 0.7955 | 1.6165 | 0.7155 | 0.6765 | 0.2476 | 0.7176 | 1.0095 | 0.3485 | 1.4465 |
| BAG4 | 1.0855 | −1.5195 | −0.4995 | 0.4055 | 0.6885 | −4.6035 | −2.1585 | −4.5575 | −3.4275 |
| BAI2 | 0.549 | 1.855 | −1.082 | 1.538 | 0.323 | −0.679 | 0.115 | 0.725 | 0.182 |
| BAMBI | −0.957 | −0.802 | 0.291 | −0.082 | −0.414 | −0.604 | −0.031 | −0.03 | 0.024 |
| BCAS1 | 0.72 | 1.883 | 1.931 | 1.05 | 1.357 | 3.983 | 3.418 | 2.961 | 1.929 |
| BCL2L14 | 7.9475 | 5.2415 | −0.8255 | 4.5295 | 0.1275 | 3.8295 | 7.6475 | 4.8305 | 5.4225 |
| BCMP11 | 4.893 | 2.389 | −1.783 | 2.166 | 0.128 | 1.042 | 4.716 | 2.067 | 3.094 |
| BEX1 | −1.3565 | −2.1385 | 0.8095 | 2.2765 | 0.8845 | 0.7765 | 1.8675 | 0.4415 | 0.8615 |
| BEX2 | −1.3785 | −1.0475 | 0.5165 | 0.3975 | −0.6165 | 0.8835 | −1.4965 | 0.1505 | 0.0415 |
| BF | −0.085 | −0.568 | 1.148 | 0.664 | −0.056 | −0.956 | 0.664 | 0.136 | −0.291 |
| BFSP1 | −1.031 | −0.216 | −0.798 | −0.313 | 0.003 | −0.438 | −0.386 | −0.194 | −0.424 |
| BIK | 0.604 | 0.32 | 1.822 | 0.287 | 1.037 | 1.823 | 0.883 | 0.734 | 0.688 |
| BIRC2 | −0.2135 | 0.3595 | 0.1735 | 0.0885 | −0.4005 | 0.5155 | 0.1585 | −0.1995 | −0.4695 |
| BIRC5 | −0.9555 | 0.6095 | −1.3395 | −0.3035 | 0.0415 | 0.8375 | 0.3135 | −0.4595 | 0.5215 |
| Bit1 | 0.3595 | 0.3185 | 0.6205 | 1.8085 | 1.0205 | 0.4335 | 1.1045 | 0.1905 | −1.6945 |
| BIVM | −0.222 | −0.35 | −0.484 | 0.125 | 0.595 | 2.613 | 0.141 | 0.155 | 0.95 |
| BLVRA | −0.85 | −0.405 | −0.985 | −0.923 | −0.701 | −0.734 | −1.278 | −0.128 | 2.323 |
| BM039 | 1.128 | −1.144 | −0.539 | −0.449 | −0.241 | −1.189 | −1.244 | −0.907 | −0.583 |
| BMP7 | 0.427 | 0.24 | 1.281 | 0.287 | 1.18 | 1.776 | 1.396 | 0.611 | 0.856 |
| BOC | 1.1805 | 1.0485 | 1.6945 | −0.7945 | −0.3635 | 0.8515 | 2.8745 | 3.7655 | 2.7115 |
| BRCA1 | 0.1415 | −0.1545 | 0.0685 | −0.1515 | 0.0735 | −0.7285 | 0.0525 | −0.5145 | −0.4105 |
| BRF2 | 0.315 | 0.495 | 0.087 | 0.236 | 0.22 | 0.247 | −0.425 | 0.693 | 0.349 |
| BRI3 | 1.809 | 0.809 | −0.355 | −0.09 | 0.42 | −0.565 | −0.338 | −0.844 | 0.31 |
| BRI3BP | 0.094 | −0.196 | 0.742 | 0.785 | 0.24 | 1.139 | 0.869 | 0.894 | 0.348 |
| BSPRY | 0.1935 | −0.9946 | −0.9915 | −1.4715 | 0.8445 | 2.5105 | −1.7725 | 1.1735 | −1.6275 |
| BTF3 | −1.0895 | −0.2565 | 0.1255 | 0.0965 | −0.3185 | −0.1065 | 0.5025 | 0.5056 | 0.2355 |
| BTG3 | 0.126 | 0.531 | 0.585 | 0.031 | 1.222 | 0.796 | 0.482 | −0.188 | 0.762 |
| BγN2A2 | 0.552 | 0.984 | 0.272 | 0.849 | 0.917 | 1.168 | 2.037 | 0.68 | 0.986 |
| BUB1 | −0.4725 | −1.2855 | −0.7175 | −0.4525 | −0.4485 | 0.3085 | −0.5355 | −0.2525 | −0.9005 |
| BUB3 | 0 | −0.471 | 0.382 | −0.549 | 0.079 | −0.218 | 0.053 | 0.029 | −0.201 |
| BXDC1 | −1.015 | 0.947 | −0.112 | 0.695 | 0.046 | 0.437 | 0.135 | −0.406 | −0.246 |
| BZRP | 1.468 | −0.914 | 1.225 | 0.534 | −0.308 | 0.447 | 1.173 | 1.329 | −1.895 |
| BZW1 | −0.9995 | −0.2145 | −1.2385 | −0.2655 | −0.3765 | 0.1545 | −0.9995 | −0.4405 | 0.1655 |
| C10orf116 | −0.4985 | −0.1785 | −0.1945 | 0.2915 | 0.1555 | 0.3505 | 0.9665 | 0.2045 | 0.2285 |
| C10orf38 | −0.418 | −0.748 | −0.48 | 0.834 | −0.294 | −1.82 | 0.623 | 0.02 | −0.74 |
| C10orf4 | 0.2835 | 0.0895 | 0.6265 | 0.3865 | −0.0535 | 0.1345 | 0.3905 | 0.0025 | −0.4065 |
| C10orf45 | 2.3895 | 1.0145 | 3.1615 | 1.4425 | 1.8855 | 3.3705 | 2.7275 | 0.8715 | 2.6785 |
| C10orf7 | 0.2665 | 0.1875 | 1.1225 | −0.6265 | 0.3135 | 0.1425 | 0.8776 | −0.5325 | −0.2595 |
| C10orf82 | 1.432 | −0.428 | 0.088 | −0.502 | 0.156 | −1.761 | −1.584 | −0.488 | −0.058 |
| C11orf24 | 1.2575 | 0.7465 | 0.1995 | −0.9055 | 0.6385 | 1.8125 | 2.7465 | −0.9455 | 1.8185 |
| C12orf14 | −0.999 | −0.515 | −0.127 | −1.038 | −0.7 | −0.219 | −0.54 | −0.771 | −0.524 |
| C13orf21 | −0.684 | 0.478 | −0.034 | 0.085 | 0.602 | 0.457 | 0.265 | 0.332 | 0.074 |
| C13orf23 | 0.008 | 0.346 | 0.508 | 0.048 | 1.008 | 1.825 | 1.015 | 0.652 | 0.87 |
| C14orf94 | 1.32 | −0.82 | 0.76 | 1.94 | 1.17 | 2.137 | 1.072 | 0.58 | −0.385 |
| C15orf23 | 0.0035 | 0.5855 | 0.9285 | 0.0475 | −0.3115 | 0.0575 | 0.8835 | 0.8095 | 0.8755 |
| C16orf45 | −1.067 | −0.672 | 2.388 | −1.219 | 0.979 | −0.132 | 0.478 | 1.107 | 1.731 |
| C17orf37 | −0.094 | 0.448 | −0.418 | 0.167 | 0.098 | −0.028 | −0.028 | 0.302 | 0.019 |
| C18orf1 | −0.483 | −0.57 | −0.848 | −0.257 | −0.459 | 0.121 | −1.831 | 0.73 | −0.052 |
| C19orf10 | 1.9415 | 2.5205 | 1.6335 | 0.8785 | 1.1805 | 1.4115 | 1.6375 | 0.1885 | 0.5955 |
| C19orf32 | 1.0755 | −0.5625 | −0.5875 | 0.1035 | 0.4775 | −0.9285 | −1.1945 | 1.5255 | −1.7095 |
| C19orf33 | 0.751 | 2.602 | 1.4 | 0.829 | 1.468 | 1.359 | −0.473 | 0.45 | −0.824 |
| C1orf24 | −0.95 | −0.622 | −0.415 | 0.24 | 0.017 | 0.2 | −0.245 | 0.052 | 0.515 |
| C1orf34 | 1.1485 | 1.1785 | 0.3705 | 0.0345 | 0.5355 | 0.1445 | 0.3995 | 0.7875 | −0.2085 |
| C20orf103 | −0.3825 | 0.4495 | 0.5065 | 0.1395 | 0.1915 | 0.7815 | 0.4315 | 0.3975 | 0.1545 |
| C20orf149 | −0.719 | 0.288 | −0.043 | 0.963 | −0.499 | −0.176 | −0.421 | 0.076 | 0.656 |
| C20orf23 | 1.722 | −0.106 | 1.096 | −0.169 | 0.297 | 0.008 | 0.706 | 0.857 | −0.248 |
| C20orf3 | 0.143 | 0.801 | 0.13 | 0.223 | 0.045 | 0.055 | 0.573 | −0.177 | −0.307 |
| C20orf35 | −0.281 | −0.55 | −0.281 | 0.228 | 0.362 | 1.048 | −0.331 | −0.379 | 0.241 |
| C20orf43 | 0.1245 | −0.0325 | 0.0645 | −0.3595 | 0.3355 | 0.7705 | 0.2605 | 0.3225 | −0.0085 |
| C20orf8 | 0.35 | 0.284 | 0.784 | 0.546 | 1.177 | 1.158 | 1.289 | 1.12 | 0.416 |
| C21orf45 | −0.323 | −0.726 | 0.343 | 0.336 | −0.285 | 0.361 | 0.031 | −0.213 | −0.863 |
| C22orf18 | −0.366 | 0.952 | 0.183 | −0.185 | 0.457 | −0.128 | 0.707 | −0.038 | 0.872 |
| C2F | −0.002 | −0.934 | 0.643 | 1.348 | 0.243 | 0.218 | 0.836 | 0.49 | −0.648 |
| C2orf17 | −1.501 | −1.708 | −0.263 | 0.174 | 0.135 | 1.424 | 0.047 | −0.697 | −0.058 |
| C4A | −0.146 | −1.025 | 0.001 | 0.006 | −0.029 | 1.3 | 0.574 | 0.157 | 0.133 |
| C5orf13 | −0.184 | −0.95 | 0.352 | 0.247 | −0.472 | 0.421 | −0.008 | −0.13 | 0.644 |
| C6orf108 | −0.602 | −1.488 | −1.828 | 0.246 | −0.348 | −0.11 | −0.693 | 0.12 | −0.248 |
| C6orf166 | 2.688 | 1.645 | 3.815 | 1.856 | 1.896 | 0.995 | 1.403 | 0.148 | −0.613 |
| C6orf192 | 0.0275 | 0.1765 | 0.1835 | −0.3395 | −0.6875 | 0.6765 | 0.6835 | −0.0385 | −0.0555 |
| C6orf211 | −0.46 | −0.245 | −0.674 | −0.343 | −0.364 | −0.322 | 0.123 | −0.225 | 0.862 |
| C6orf49 | −0.7216 | 0.0875 | −0.1185 | 0.1865 | 0.3585 | 1.1595 | 0.0695 | −0.8745 | −0.1235 |
| C6orf85 | 1.8705 | 0.6355 | 0.2625 | 0.2605 | 0.5545 | −0.4355 | −0.4645 | −0.2065 | 0.4005 |
| C7orf24 | 2.501 | 1.142 | 3.159 | −0.208 | 2.333 | 0.338 | −1.035 | 2.315 | −0.257 |
| C9orf10 | −0.118 | −0.159 | −0.839 | −0.43 | −0.522 | −2 | −2.30 | −1.896 | −1.461 |
| C9orf121 | 0.994 | 0.504 | −0.26 | 0.07 | 0.124 | −1.172 | −0.028 | −0.684 | 0.098 |
| C9orf58 | 0.8955 | −0.0685 | −0.0305 | −0.1845 | −0.2575 | 0.0595 | −0.7145 | −0.4875 | −0.4176 |
| C9orf88 | 2.758 | 2.037 | 1.706 | 0.728 | 1.503 | 1.915 | 0.16 | 1.412 | 1.38 |
| C9orf9 | 2.325 | 0.297 | 1.949 | −0.919 | 0.868 | −1.651 | 0.076 | 0.963 | 0.38 |
| CA12 | 2.139 | 0.193 | 1.963 | −1.432 | 1.143 | −0.404 | 0.123 | 0.784 | 0.036 |
| CA8 | 0.454 | −0.318 | −0.027 | −0.329 | −0.128 | −0.17 | 0.012 | 0.051 | −0.223 |
| CA8 | 0.5925 | 0.2055 | 0.1085 | −0.1375 | −0.0295 | 0.5585 | −0.2535 | −0.2505 | 0.2385 |
| CACNA1G | −0.56 | −0.41 | −0.236 | −0.436 | 0.098 | 0.782 | −0.74 | −0.393 | 0.259 |
| CALCA | 0.256 | −0.104 | 2.96 | −0.431 | −0.718 | −1.115 | −0.467 | −0.702 | −1.095 |
| CALML4 | 1.6385 | 0.6415 | −0.0785 | 1.2495 | 1.7945 | −1.5315 | 1.8635 | 0.7485 | 2.3205 |
| CALML5 | 1.2705 | 1.3065 | 0.8095 | 1.0385 | 0.5165 | 0.5185 | −1.2725 | −0.4875 | −0.4665 |
| CaMKIINa [symbol] | −0.055 | −0.147 | 0.169 | 0.09 | 0.24 | 0.597 | 0.411 | −0.436 | −0.095 |
| CAP2 | 0.287 | 0.113 | −1.035 | 0.125 | 1.276 | −0.557 | 1.814 | −0.772 | 1.905 |
| CAP350 | −0.7465 | 0.1645 | −1.4035 | −0.9245 | −0.1355 | −0.6055 | −1.7485 | −0.2635 | −0.4235 |
| CAPN13 | −0.178 | 0.559 | 1.314 | −0.853 | −0.446 | 0.235 | 0.433 | −0.196 | −0.221 |
| CAPN9 | 1.3095 | −0.0555 | 0.1075 | 0.1825 | 0.1235 | 2.1135 | 0.0635 | 3.7865 | −0.2105 |
| CARD14 | −1.9335 | −1.4265 | 0.1925 | −0.0875 | 0.3505 | −0.1265 | −0.8935 | −1.4715 | −0.5625 |
| CART | −0.7395 | −0.0285 | −0.1615 | 0.0765 | 0.0195 | 0.5025 | 0.1595 | 0.2935 | 0.4095 |
| CASP1 | −1.109 | −0.795 | 0.645 | 0.5 | 0.831 | 0.34 | 0.395 | 1.594 | 0.368 |
| CASP3 | −0.005 | 0.098 | 0.49 | −0.149 | −0.232 | −0.215 | −0.167 | 0.104 | 0.119 |
| CASP7 | 0.0745 | −0.6055 | −0.4455 | −1.2955 | 0.0145 | −1.9955 | 0.5775 | −1.8055 | −0.4115 |
| C9LN1 | −1.204 | −0.403 | −2.453 | −1.461 | −0.757 | −1.725 | −0.165 | −0.414 | −0.825 |
| CBR1 | −1.2395 | −0.4405 | 0.3285 | 0.8075 | 0.0795 | 0.5005 | −0.7065 | −0.1945 | −0.5755 |
| CBR3 | 0.001 | −0.083 | −0.127 | −0.235 | −0.029 | −0.875 | −0.227 | −0.185 | −0.358 |
| CBR4 | −0.5195 | −0.6325 | −2.0565 | 0.1425 | −0.3285 | −0.4485 | −2.5895 | −2.5455 | −1.3745 |
| CCL24 | −0.44 | 0.295 | 0.121 | −0.58 | 0.808 | 1.263 | 0.236 | 0.629 | 1.063 |
| CCL29 | 3.187 | 0.933 | 2.782 | −0.09 | 3.913 | 0.166 | 2.437 | 2.887 | 2.026 |
| CCNB2 | −0.1235 | −0.1485 | −0.8116 | −0.3305 | 0.8785 | 0.3415 | −0.0735 | 0.2645 | −0.0495 |
| CCND1 | 0.275 | −0.066 | 0.398 | 0.385 | 1.053 | −0.035 | 0.238 | 0.752 | −0.88 |
| CCNE1 | 0.1995 | −0.0175 | 0.5155 | 0.5255 | 0.4715 | 1.0715 | 1.1775 | 0.8045 | 0.6035 |
| CCNG2 | −0.472 | −0.412 | 0.768 | −0.917 | 0.212 | −1.25 | −0.945 | −0.435 | −1.572 |
| CCNH | −1.1975 | −1.0335 | 0.6425 | −0.6495 | −0.0085 | −1.7385 | −0.8875 | −1.3405 | −0.4745 |
| CD38 | 0.0945 | −0.1665 | 0.2055 | −0.7245 | −0.4805 | −0.9715 | −0.6185 | −0.4505 | −0.4325 |
| C030 | −0.7575 | −1.1335 | −0.8895 | 0.2535 | 0.0115 | 0.7645 | −0.4775 | −0.6235 | −0.9465 |
| CD4 | −0.8075 | −0.1615 | −0.2775 | −1.1215 | −0.4045 | 0.4805 | −0.5255 | 0.6235 | −0.1215 |
| CD58 | 0.089 | 0.019 | 0.462 | 0.582 | 1.5 | 2.209 | 1.363 | 0.308 | 0.751 |
| CD50 | 0.8545 | −0.0165 | 0.8025 | 0.0285 | 1.3715 | 1.5705 | 1.4535 | 1.0135 | −0.2395 |
| CDC2 | 0.2745 | 0.5275 | 0.6505 | 1.1525 | 1.1635 | 1.8685 | 0.9685 | −0.2555 | 0.3545 |
| CDC6 | 0.2395 | 1.4075 | 0.8905 | −0.3605 | 0.4595 | 1.2515 | 1.5225 | 0.1575 | 0.4475 |
| CDCA1 | −1.847 | −0.267 | −0.608 | −1.383 | −0.113 | 1.314 | 0.817 | −0.386 | 0.476 |
| CDCA5 | −2.101 | 0.737 | 0.257 | 0.933 | −2.503 | 1.121 | 0.577 | 0.394 | 1.166 |
| CDCA7 | −1.3785 | −0.0985 | −0.2615 | −0.7625 | −0.4475 | 0.5985 | −2.6905 | −0.9655 | −1.2065 |
| CDH1 | −0.559 | −0.014 | 0.014 | 0.099 | 0.447 | 0.329 | 0.015 | 0.886 | 0.328 |
| CDH3 | 0.0715 | 0.4255 | −0.0825 | −0.8125 | 0.2565 | 0.3275 | −0.9855 | −0.0175 | 0.3565 |
| CDK2 | 1.5775 | 1.3435 | −0.0365 | 0.2095 | 1.3115 | 1.4345 | 1.6385 | 1.5155 | 1.2085 |
| CDKN2A | 0.1795 | −0.6565 | 0.8185 | 0.5415 | 0.3885 | 1.9615 | 0.8345 | 0.3735 | 0.7135 |
| CDKN2D | −0.387 | −0.61 | 0.764 | −0.107 | 0.456 | 1.199 | 0.518 | −0.011 | −0.154 |
| CDKN3 | 0.053 | 0.688 | −0.534 | −0.128 | 0.287 | 0.31 | 0.742 | 0.933 | 0.577 |
| CDT1 | 1.1285 | 0.6215 | 1.1295 | 1.6315 | 1.5075 | 1.0165 | 0.7235 | 0.9195 | −0.8685 |
| CE9PG | 0.038 | −0.079 | 0.017 | −0.129 | 0.483 | 0.148 | 0.158 | −0.322 | 0.128 |
| CELSR1 | −0.324 | 0.377 | 0.542 | 0.246 | 1.213 | 0.914 | −0.306 | 0.77 | 0.824 |
| CENPA | −0.014 | 0.008 | 0.542 | 0.262 | 1.326 | 1.271 | 0.124 | −0.148 | 0.072 |
| CENPE | 0.108 | 0.306 | −0.288 | −0.247 | −0.23 | −1.3 | 0.029 | −0.428 | 0.407 |
| CENPF | 0.4125 | 0.0095 | −0.1815 | −0.0995 | −0.2825 | 0.6245 | −0.1835 | 0.6785 | 0.4175 |
| CENTG3 | 0.4545 | 0.3285 | 0.2655 | 0.5695 | 0.6045 | 2.1425 | 1.1415 | 0.7145 | 0.6425 |
| CES2 | 1.862 | 3.191 | 0.123 | 0.714 | −0.175 | 1.196 | 1.152 | 3.936 | 1.14 |
| CETN3 | 0.2665 | 1.2275 | 1.0035 | −0.5795 | 0.6215 | 0.9495 | 1.3185 | 0.6055 | 0.3935 |
| CGA | −0.3915 | −0.4305 | 1.2615 | 0.7565 | 0.4795 | 0.9555 | 0.6475 | 0.2125 | 0.0065 |
| CGI-12 | 0.4675 | 0.4255 | 1.6425 | 1.7745 | 1.3005 | 1.4825 | 2.0285 | 0.4965 | −1.4235 |
| CGI-49 | 1.452 | 1.293 | 2.052 | 1.021 | 1.039 | 0.888 | 0.688 | 1.007 | 0.148 |
| CGI-4 [symbol] | −1.21 | −0.13 | −1.752 | −0.979 | 1.005 | −0.646 | −2.163 | 2.531 | −1.506 |
| CGN | −0.012 | −0.342 | 0.105 | −0.472 | 0.478 | 1.213 | 0.973 | 0.826 | 0.603 |
| CGNL1 | 0.1405 | 0.6405 | 0.6235 | −0.0575 | 0.4885 | 0.1455 | −0.4345 | 0.6475 | 0.4985 |
| CHAF1B | 0.17 | −0.028 | 0.41 | 0.176 | 0.514 | 1.038 | 0.834 | −0.161 | 0.352 |
| CHC1 | 0.47 | 0.498 | 1.117 | 0.51 | 0.833 | 0.39 | 0.516 | 0.411 | 0.333 |
| CHCHD2 | −0.635 | −0.346 | −0.603 | −1.369 | −0.019 | 0.697 | −1.452 | −1.471 | −0.785 |
| CHCH05 | −2.166 | −1.871 | −1.441 | 2.715 | −1.554 | −1.186 | −1.146 | −2.026 | −2.631 |
| CHI3L1 | 0.525 | −0.291 | 2.316 | −0.866 | 0.11 | −0.024 | 0.601 | −0.527 | −0.583 |
| CHI3L2 | −0.045 | −0.066 | 0.122 | −0.351 | −0.221 | −0.178 | −0.298 | 0.127 | 0.199 |
| CHKA | −0.042 | 0.336 | −0.654 | −1.004 | 0.227 | 0.794 | −0.191 | −0.196 | 0.006 |
| CHCDL | 0.1565 | −0.0935 | −0.0485 | −0.3685 | −0.3165 | −0.7115 | −0.4845 | −0.0865 | −0.2255 |
| CHRDL2 | 0.042 | −0.534 | 0.508 | 0.474 | −0.324 | 0.597 | 0.696 | −0.047 | −0.029 |
| CHST2 | −0.3475 | 0.2705 | 0.1765 | 0.0835 | 0.1015 | −0.4045 | 0.6935 | −0.1015 | 0.2915 |
| CHURC1 | −0.118 | 0.181 | −0.02 | 0.591 | −0.012 | −0.286 | 0.407 | 0.472 | 0.281 |
| CIR | −0.1905 | −0.4705 | −0.3165 | 0.3035 | 0.2205 | 1.4355 | 0.0295 | −0.4805 | 0.2455 |
| CIRBP | −1.0505 | −0.9525 | 0.1145 | 0.7555 | 0.2165 | −0.1485 | 0.1625 | −0.3395 | −0.3745 |
| CKLF | −0.7675 | −1.3635 | −0.7565 | 0.1285 | 0.4155 | −1.4205 | −2.2455 | −0.0785 | −0.9135 |
| CKLFSF6 | −1.4825 | 0.1425 | −1.5235 | −0.3775 | 0.7255 | 0.4345 | 1.6576 | 1.0525 | 1.7775 |
| CKLFSF7 | 0.21 | 0.208 | 0.728 | −0.111 | 0.999 | 1.841 | 0.944 | −0.63 | 0.3 |
| CKMT1 | 1.263 | 0.036 | 0.919 | −0.105 | 1.109 | 1.648 | 0.782 | −0.064 | 0.596 |
| CKS1B | −0.332 | 0.51 | 0.194 | 0.433 | 1.036 | 1.482 | 0.739 | 0.935 | 0.849 |
| CKS2 | 0.4675 | 1.1805 | −0.2525 | −1.3215 | −0.5765 | −0.1995 | 0.3025 | −0.7915 | 0.3495 |
| CL640 | 0.6915 | 0.8545 | 1.8655 | −0.1455 | −0.4885 | 0.1165 | −0.6855 | −2.685 | −0.5795 |
| CLDN23 | 0.3475 | 0.6885 | 0.1785 | −0.5015 | −0.6275 | −1.1445 | 0.0895 | −0.2165 | −0.5965 |
| CLDN3 | −0.7055 | −0.6585 | −0.6925 | −0.9135 | 0.3275 | 0.2115 | 0.2005 | 0.4575 | −1.1865 |
| CLDN4 | 0.954 | 0.185 | 2.387 | 2.417 | 0.774 | 1.253 | 2.862 | 1.599 | −1.134 |
| CLDN8 | 3.3135 | 2.1895 | −0.7815 | 0.9795 | 1.3195 | 0.2875 | 0.1055 | −0.3735 | −0.5925 |
| CLGN | −1.0145 | −0.9765 | −0.3525 | −0.2325 | 0.0575 | 1.0495 | 1.7955 | 4.0375 | −0.0735 |
| CLIC6 | 0.33 | 0.867 | 0.688 | 0.914 | 0.024 | 0.326 | −0.088 | −0.027 | −0.208 |
| CLNS1A | −0.1885 | −0.2525 | −0.0945 | −2.0895 | −0.7195 | −0.8475 | −0.1945 | 0.5295 | −0.9275 |
| CLTC | 1.077 | −0.408 | 0.235 | 0.598 | 0.062 | 0.118 | 0.268 | 0.352 | 0.439 |
| CLU | 0.229 | 0.156 | 1.263 | −0.633 | 0.264 | 1.14 | 0.85 | −0.158 | 0.12 |
| CMAS | 0.0625 | −0.8715 | 0.0905 | 0.7265 | −0.4495 | 0.9565 | 0.4715 | −0.1195 | 0.2795 |
| CML65 | −0.331 | −1.06 | 0.489 | 0.172 | 0.256 | 0.734 | 0.641 | −0.052 | 0.289 |
| CNIH | 0.137 | 0.336 | −0.145 | 0.337 | 0.028 | −0.432 | −0.051 | 0.352 | 1.153 |
| CNOT10 | 0.0945 | 0.8525 | −0.3145 | −0.4375 | −0.1275 | 0.9565 | −0.3475 | −0.2185 | 0.1265 |
| CNOT2 | −0.007 | 0.319 | 0.054 | 0.549 | 0.285 | −0.158 | 0.072 | 0.004 | 0.506 |
| CNOT7 | 0.623 | −0.8 | −2.79 | −0.622 | 1.654 | 1.899 | 3.015 | 4.159 | 3.758 |
| CNP | 0.4885 | 0.6775 | 1.4015 | 0.6235 | 0.0735 | 0.1665 | 0.1275 | 0.4545 | 0.2025 |
| CNTNAP2 | 0.024 | 0.046 | −0.157 | −0.256 | −0.25 | −0.425 | −0.441 | 0.084 | 0.676 |
| COASY | −0.6985 | 0.3895 | −1.7185 | 0.1995 | −0.4955 | −2.2085 | −1.7065 | −1.6365 | −0.7425 |
| COL11A2 | −1.143 | −1.055 | 0.385 | 1.188 | 0.787 | 1.466 | 0.253 | 0.385 | −0.048 |
| COL27A1 | 0.261 | 0.889 | −0.713 | 0.023 | −0.058 | −0.357 | −0.83 | 0.264 | −0.065 |
| COL4A5 | −1.3335 | −0.8365 | −2.2555 | −1.5895 | −0.9655 | −1.8465 | 0.1465 | −0.7585 | −0.9345 |
| COL8A1 | 0.006 | −0.221 | 1.317 | 0.452 | 0.165 | 2.106 | 0.879 | −0.415 | 0.334 |
| COL8A3 | 0.082 | 1.52 | 1.517 | −0.28 | 0.226 | 0.237 | 0.654 | 0.58 | 0.53 |
| COMMD1 [symbol] | 0.4645 | 0.2175 | −0.0375 | 0.2805 | 0.1925 | −0.3485 | 0.1135 | 0.0695 | −0.1645 |
| COMMD5 | −0.0055 | −0.1815 | −0.3465 | −0.2845 | 1.1205 | 0.0745 | 1.1355 | 1.2315 | 2.2965 |
| COPS7B | 0.281 | 0.229 | 0.384 | −0.033 | 0.101 | 0.219 | −0.312 | 0.595 | 0.813 |
| CORO6 | 0.6815 | 0.9725 | 0.9275 | 0.0905 | 0.2415 | 1.0825 | 1.1365 | 0.7205 | 1.1745 |
| CORT | 0.179 | −0.11 | −0.162 | −0.659 | 0.101 | 0.599 | 0.64 | 0.396 | 0.101 |
| COX17 | 0.249 | 0.341 | −0.197 | −0.695 | 0.227 | 0.949 | 0.644 | 0.57 | 0.561 |
| COX5A | 0.147 | 0.923 | 1.143 | 1.196 | 0.104 | 1.051 | 0.748 | 1.191 | 1.297 |
| COX6A | 5.4785 | 4.3545 | 1.7495 | 0.2975 | 2.4315 | 4.6095 | 4.6455 | 2.3155 | 3.6565 |
| COX8A1 | −0.2535 | −0.9555 | 0.4755 | 0.2705 | −0.7225 | 0.6355 | −0.0375 | 0.8545 | −0.2195 |
| COX8C | −1.7755 | −1.4425 | 3.1335 | −1.0625 | −0.5925 | 3.9965 | −1.5315 | −1.1355 | −1.0375 |
| COX7A2 | −0.14 | −0.092 | −1.096 | −0.73 | −0.105 | 0.876 | −1.361 | −0.255 | −0.996 |
| CP | 2.3475 | −0.0025 | 1.9165 | 0.3865 | −0.2595 | 1.6885 | 3.7625 | 2.7685 | −0.0935 |
| CPAMD0 | −0.013 | 0.482 | 0.351 | 0.193 | 0.189 | −0.241 | 0.758 | 0.682 | 0.765 |
| CPB1 | 1.3435 | 1.2525 | 0.7085 | −0.2305 | 0.8955 | 0.3815 | −0.4605 | 0.3795 | 1.4645 |
| CPEB3 | −0.844 | −0.283 | −0.026 | −0.105 | 0.053 | −0.044 | 0.144 | 0.233 | −0.552 |
| CPEB4 | 0.997 | 0.154 | −1.046 | −0.798 | −0.463 | −0.746 | −1.04 | −0.416 | 0.697 |
| CPSF3 | −0.8425 | 0.2015 | −0.2865 | 0.2345 | 0.0055 | −0.2535 | 0.4075 | 0.2595 | −0.3465 |
| CRABP1 | −0.993 | 0.466 | 0.982 | 0.193 | −0.031 | 0.371 | 0.598 | 0.439 | 1.018 |
| CREB3L4 | 0.897 | 0.153 | 0.099 | −0.426 | −0.28 | 1.096 | −0.835 | −0.226 | 0.237 |
| CRIP1 | 0.613 | 0.506 | −0.248 | 0.347 | −0.297 | 0.224 | 1.716 | 0.487 | 0.712 |
| CRYBB2 | 0.422 | 1.544 | 0.538 | 1.046 | 0.664 | 0.308 | 1.209 | −0.338 | 0.949 |
| CRYL1 | −0.732 | −0.397 | 0.365 | −0.723 | −0.359 | −0.604 | −2.02 | −0.015 | −0.643 |
| CRYZ | −0.1275 | 1.2555 | 0.2815 | 0.0665 | 0.8225 | 0.9155 | 1.3755 | 0.3715 | 0.0865 |
| CSDA | 0.1965 | 0.0275 | 0.1245 | −0.4035 | −0.2165 | −0.4915 | −0.3485 | −0.1205 | −0.3525 |
| CSE1L | 0.3355 | 0.9015 | −0.6255 | −0.2675 | −0.6015 | −1.5225 | −0.6375 | −0.9475 | 0.0515 |
| CSK | −1.5775 | −1.2445 | 1.3885 | −0.4445 | 0.2005 | −1.3455 | −2.5065 | −0.8725 | −0.2705 |
| CSRP1 | −0.3315 | −0.5215 | −0.9475 | −1.0085 | −0.1595 | 1.7025 | −1.1795 | −1.7445 | 2.3035 |
| CSRP2 | 0.576 | −0.055 | −0.387 | −0.502 | −0.256 | −0.059 | 0.209 | 0.09 | −0.033 |
| CST5 | −0.3335 | −0.0185 | 0.1715 | −0.7715 | 0.0915 | −1.0735 | −1.3015 | 0.0185 | −0.0555 |
| CSTB | −0.316 | 0.399 | 0.211 | 0.895 | 0.694 | −0.027 | 0.107 | −0.35 | 0.609 |
| CTAG1B | 0.1855 | −0.3595 | 0.4865 | −0.2035 | 0.0775 | 0.2845 | 0.1585 | −0.0115 | −0.1655 |
| CTBP2 | −0.274 | −1.065 | 0.017 | −0.271 | 0.228 | 0.479 | 1.017 | −0.18 | 0.181 |
| CTNNA1 | 3.152 | 0.427 | 2.779 | −1.832 | 1.142 | −0.508 | 0.849 | 0.294 | −0.209 |
| CTNNAL1 | −0.7375 | 0.2275 | 0.3685 | −0.2985 | 0.3125 | −0.1685 | −0.5525 | −0.5405 | 0.5685 |
| CTNND2 | 0.345 | −0.103 | −0.412 | 0.767 | 1.442 | 1.655 | −0.044 | −0.999 | −0.432 |
| CTPS | −1.075 | −0.699 | 0.084 | 0.331 | −0.182 | −0.27 | −0.639 | −0.965 | −0.433 |
| CTSL2 | −0.0315 | 0.2135 | 0.2105 | −0.7215 | 1.6885 | −0.8855 | 1.7545 | 1.6435 | 1.8545 |
| CTSS | −1.4 | −0.54 | −1.78 | −1.12 | −1.31 | −2.937 | 0.149 | −1.56 | 2.407 |
| CTTN | −0.9335 | −2.0295 | 2.6245 | −0.7345 | −0.5235 | −1.0205 | −0.5285 | −1.9085 | −1.1335 |
| CX3CL1 | −0.5165 | −0.0835 | 0.2085 | 1.4045 | 0.3455 | 1.6535 | 0.9125 | 0.4375 | 0.4605 |
| CXCL9 | 0.4085 | −0.3415 | −0.1005 | −0.3935 | 0.5735 | 1.7665 | 1.9975 | 2.0075 | 1.6405 |
| CXorf39 | −1.196 | 0.655 | 0.403 | 0.034 | 0.856 | 1.003 | 0.291 | −0.134 | 0.527 |
| CXXC4 | 1.78 | 1.48 | 0.436 | 0.403 | −0.194 | 0.093 | −0.012 | 0.308 | 0.648 |
| CYB5 | 0.8515 | 1.2745 | 1.9085 | −0.5175 | 0.5565 | 0.4945 | 0.4315 | 0.1465 | 0.7755 |
| CYB561 | 0.683 | 1.147 | 1.483 | −0.316 | 0.516 | 0.004 | 0.452 | 0.534 | 0.348 |
| CYC1 | −0.153 | −0.069 | −0.169 | −0.203 | −0.304 | 0.136 | 0.011 | 0.314 | 1.027 |
| CYHR1 | 0.1515 | 0.0015 | 0.4335 | −0.3835 | 1.5075 | −0.4445 | 3.2125 | −1.4305 | 1.1895 |
| CYP2A13 | −0.225 | −0.06 | −0.605 | 0.049 | 0.034 | −0.831 | 0.562 | 0.659 | −0.106 |
| CYP4X1 | 1.5895 | 0.1655 | 0.1745 | 1.2605 | 0.2765 | 2.2395 | 1.5385 | 1.4725 | 0.9855 |
| D15Wsu7 [symbol] | 0.5745 | 0.0055 | 0.2705 | 0.2275 | 0.3595 | 2.0505 | 1.6915 | 1.0405 | 0.5505 |
| DACH1 | 1.952 | 1.159 | 0.48 | 1.21 | 0.741 | 2.269 | 2.066 | 1.836 | 1.633 |
| DACH1 | −0.298 | 0.188 | 0.569 | 0.75 | 0.025 | 0.406 | 0.174 | −0.221 | −0.112 |
| DACH1 | −0.971 | −0.407 | −0.533 | 0.9 | −0.187 | −0.003 | 0.701 | −0.397 | −0.691 |
| DAP3 | −0.1825 | −0.1775 | 0.2965 | 0.8875 | 0.2735 | 0.4525 | 0.9505 | 0.6505 | 0.2975 |
| DAPP1 | −1.539 | −0.676 | −0.129 | 0.647 | 0.808 | 0.327 | −0.168 | 0.339 | −0.469 |
| DAZAP2 | 0.495 | −0.926 | −0.095 | −0.426 | −0.204 | 0.166 | −0.294 | −1.442 | −0.339 |
| DBI | −0.5695 | −0.1595 | −0.5585 | −0.1285 | −0.2915 | 1.2015 | −0.4385 | −0.2875 | 1.1415 |
| D9N1 | −0.852 | −0.905 | −0.363 | −0.219 | −0.407 | −0.162 | −0.426 | −0.623 | −0.557 |
| DC13 | −0.0265 | −0.0035 | 0.5875 | −0.1615 | −0.2245 | 0.6035 | 2.0045 | 2.2175 | 2.2185 |
| DCPS | 1.5795 | −0.7525 | −0.6595 | −0.0255 | 0.0415 | −0.6035 | −0.1395 | −0.8116 | −0.3805 |
| DDHD2 | 0.0195 | 0.7735 | 0.3435 | −0.4015 | 0.3675 | −0.4265 | −0.0165 | −0.1725 | 0.4205 |
| D0X17 | 0.1495 | −0.0465 | 1.3275 | 0.5935 | 0.0545 | 0.5205 | 0.2135 | 0.6495 | 0.3255 |
| DDX39 | −1.689 | 0.14 | 0.273 | −1.293 | 0.283 | −1.463 | 1.564 | −0.992 | −0.517 |
| DDX48 | −0.2135 | 1.1955 | 0.1525 | 0.0405 | −0.0995 | −0.7525 | −0.0945 | 0.0555 | 0.8555 |
| DDX58 | −1.0995 | −0.6555 | −0.5715 | 0.1615 | 0.2305 | 1.1485 | 0.9735 | −0.5905 | 0.0905 |
| DEF6 | 0.538 | 0.047 | 2.861 | −0.818 | 0.77 | −0.645 | 0.718 | 4.133 | −0.466 |
| DEK | −0.876 | 0.161 | 0.68 | 0.463 | −0.268 | −0.007 | 1.003 | 0.625 | 0.176 |
| DHCR7 | −0.53 | −0.676 | −1.752 | −1.068 | −0.382 | −1.564 | −1.018 | −1.589 | 0.886 |
| DHRS7 | −0.8995 | −1.1045 | 0.3385 | −0.3865 | 0.0295 | 0.0455 | 1.1615 | −0.5325 | 0.0015 |
| DHX57 | 0.0635 | −0.6635 | 0.1585 | 0.2785 | −0.3295 | −1.1115 | 0.3875 | 0.8945 | −0.8005 |
| DIRC1 | −0.381 | 0.035 | −0.145 | −0.279 | 0.008 | 1.064 | −0.127 | 0.312 | 0.666 |
| dJ222E13 | −0.141 | −0.132 | 0.465 | 0.012 | −0.169 | 0.732 | −0.587 | 0.263 | 0.501 |
| DKFZP434 | 0.488 | 0.473 | 0.413 | −0.654 | −0.153 | −0.32 | 0.468 | −1.303 | 0.559 |
| DKFZp434 | 0.0235 | 0.5375 | −0.3955 | 0.3045 | 0.9605 | −0.3705 | −0.2205 | −0.4715 | 0.3335 |
| DKFZP434 | 0.099 | −0.419 | 1.039 | 0.07 | 0.031 | 0.27 | −0.033 | 0.444 | −0.179 |
| DKFZp434 | −0.1325 | 0.3155 | 0.8145 | 0.3255 | −0.0685 | −0.0105 | 0.0225 | 0.1805 | 0.0305 |
| DKFZP564 | −0.2845 | −0.1435 | 0.1165 | 0.5185 | 0.0985 | 0.9525 | 0.1325 | 0.5395 | −0.3535 |
| DKFZp564 | 0.031 | 0.552 | 1.399 | −0.712 | 0.689 | 0.795 | −0.551 | 0.05 | 0.151 |
| DKFZP564 | 0.4895 | 0.3885 | 0.4175 | 0.1905 | −0.0285 | 0.9465 | 0.5215 | −0.0265 | −0.1425 |
| DKFZP564 | −0.544 | −0.495 | −0.872 | −0.153 | 0.017 | 0.68 | 0.31 | 0.082 | −0.459 |
| DKFZP564 | 0.555 | −0.511 | −1.687 | −0.279 | −0.89 | −1.462 | −0.888 | −0.739 | −0.165 |
| DKFZp586 | −0.5295 | 0.3145 | −0.8035 | −0.3965 | 0.0645 | −1.6265 | −1.4735 | −0.3535 | 0.1765 |
| DKFZp761 | 0.189 | 0.193 | 0.042 | −0.289 | −0.302 | −0.417 | −0.312 | −0.323 | 0.037 |
| DLK1 | 0.034 | 1.582 | 1.42 | −1.719 | 0.638 | 0.101 | 0.396 | −2.944 | 1.649 |
| DLX5 | 0.5955 | 1.6885 | 1.3905 | 1.5955 | 1.4245 | 1.3675 | 1.6975 | 1.0045 | 0.1615 |
| DNAJA4 | 2.4505 | 3.0515 | 2.6505 | 2.6665 | 0.8625 | 0.0625 | 3.5825 | 1.7155 | 1.9285 |
| DNAJC1 | −0.277 | −0.721 | −0.238 | −0.9 | −0.217 | −1.877 | 0.881 | −0.373 | 0.318 |
| DNAJC12 | 1.687 | 1.472 | 1.528 | 1.449 | 0.877 | 1.443 | 0.635 | 1.502 | 1.158 |
| DNAJD1 | −0.065 | −0.107 | −0.152 | 0.196 | 0.664 | −0.26 | 0.186 | 0.112 | 0.238 |
| DNALI1 | −0.243 | 1.989 | −0.459 | 0.286 | 0.01 | −0.481 | −0.205 | 1.753 | −0.325 |
| DNMT1 | −1.7535 | −0.4445 | −1.6585 | −1.2215 | −0.5905 | −0.1725 | −0.9505 | −0.2535 | −0.0125 |
| DOX1 | −0.6275 | −0.0915 | −0.1355 | 0.9275 | 0.6885 | 1.1285 | −0.2885 | 0.9495 | 0.5075 |
| DSCR1 | 2.7135 | 0.7845 | 0.0185 | −0.4675 | 0.1185 | −2.6625 | −0.1675 | 0.4405 | −2.3135 |
| DSCR1L2 | 0.3095 | −0.6865 | 0.3555 | 0.3135 | 0.9975 | 0.5205 | 0.8375 | 0.0325 | 0.5235 |
| DSP | 0.0405 | 0.2995 | −0.1925 | 0.5745 | 0.4325 | 0.7585 | 0.3465 | −0.2565 | −0.3305 |
| DTYMK | 0.2085 | −0.3455 | −0.4785 | −0.5415 | −0.6565 | −1.0915 | −0.3885 | 0.0345 | −0.7735 |
| DUSP23 | −0.4805 | −0.1275 | −1.6915 | −0.8495 | −0.2095 | −1.0205 | −0.7595 | 0.2015 | −0.4825 |
| DUSP4 | −0.0225 | 0.7155 | −0.1285 | −0.1025 | 0.1035 | −0.9235 | −0.9305 | 0.0655 | 0.3275 |
| DUSP5 | 0.045 | 0.4 | −0.082 | −0.238 | −0.165 | 0.133 | 0.027 | 0 | 0.242 |
| DVL1 | −0.074 | −0.448 | 0.314 | 0.638 | 0.142 | 0.44 | 0.297 | −0.059 | −0.22 |
| DXYS155 [symbol] | 0.646 | 0.135 | 1.142 | −0.586 | 0.234 | 0.628 | 0.913 | −0.176 | −0.317 |
| DYRK4 | −1.0975 | −0.8955 | −0.3615 | −0.6315 | −0.1205 | −0.7045 | −0.6075 | −0.2075 | −0.5675 |
| o(y)2 | −0.007 | 0.177 | 0.583 | 0.287 | 0.195 | 0.312 | −0.336 | 0.147 | 0.237 |
| EAF2 | −0.4715 | −0.3785 | −0.9235 | −0.0465 | −0.2495 | −0.6005 | 0.2565 | −0.4115 | 0.0085 |
| EAP30 | 1.1345 | 0.2145 | 0.7235 | 0.2025 | 0.1655 | 1.2755 | 0.6465 | −1.0095 | −1.4895 |
| ECHDC1 | 3.0475 | 0.1645 | 0.8145 | 1.3385 | 0.9145 | 2.1435 | 2.5895 | 0.5365 | 1.7735 |
| ECHDC3 | −0.7815 | 0.5005 | 1.0625 | 0.5405 | −0.3565 | 1.1025 | −0.1635 | 0.1485 | 0.2915 |
| EEF1A2 | −0.605 | −1.064 | −0.333 | 0.785 | −0.447 | 0.759 | −1.499 | −0.803 | −2.236 |
| EFHC1 | −1.4655 | −2.3585 | −2.2795 | −2.0195 | 0.4185 | −0.2365 | −0.0445 | −2.8215 | −0.6395 |
| EFNA4 | −0.0285 | 0.4505 | 0.7845 | −0.5945 | 0.2125 | −0.0515 | −0.4055 | −0.0105 | 0.2805 |
| EFS | −0.4185 | 0.3165 | −0.1635 | 0.1175 | 0.1795 | −0.1795 | 0.1125 | 0.0485 | 0.1825 |
| EIF2C2 | −0.2895 | −0.7025 | 0.5005 | −0.9485 | −0.9075 | −0.1545 | 0.2645 | 1.9135 | 2.8265 |
| EIF2C4 | 0.517 | 0.339 | −0.03 | −0.146 | −0.218 | −0.586 | −0.75 | −0.303 | 0.161 |
| EIF4EBP1 | −0.2555 | −0.8375 | −0.0885 | 1.4835 | 0.1405 | −0.5305 | −1.3515 | −0.5315 | 0.2315 |
| EIF4G1 | −1.832 | −0.885 | −0.105 | −0.168 | 0.113 | 1.633 | 0.409 | −0.894 | 0.183 |
| EIF4G3 | 0.412 | −1.369 | −0.053 | 1.592 | −0.432 | 1.452 | 0.256 | −0.146 | −0.494 |
| ELAC1 | 0.612 | 1.758 | 0.639 | −0.753 | 0.82 | 1.574 | 1.032 | 1.121 | 0.233 |
| ELL2 | −0.3575 | 0.5155 | 1.1465 | 0.8585 | 0.7685 | 0.2575 | −0.3285 | 0.3945 | −1.3985 |
| ELL3 | −0.7045 | −0.8825 | −0.6305 | −0.2855 | −0.4276 | −0.6285 | −0.9285 | −0.4055 | −0.2385 |
| ELOVL5 | −0.7945 | −0.7526 | −0.1385 | 0.0045 | 0.0065 | 0.3545 | −0.6645 | −0.7755 | 0.1025 |
| EN1 | 0.3245 | −1.8215 | −0.5595 | −0.8515 | −0.6885 | 1.4955 | 0.7175 | 1.0055 | 0.0005 |
| ENAH | −0.8575 | −0.9155 | 0.1335 | 0.8995 | 0.4425 | −0.5395 | 0.1355 | −0.3715 | 0.3125 |
| EPHX2 | 1.133 | 0.963 | 1.444 | 0.489 | 0.039 | 1.653 | 1.18 | 0.461 | 0.773 |
| EPLIN | 0.6695 | −0.1315 | 0.0265 | 0.7525 | 0.9216 | −0.0385 | 0.9995 | −0.6835 | 0.1765 |
| EPN3 | −0.2585 | 0.2955 | 0.3125 | 0.1185 | −0.5245 | −0.6045 | −0.7845 | 0.3235 | −1.6675 |
| EPOR | −1.083 | −0.108 | −0.445 | 0.085 | −0.204 | 0.279 | −0.899 | −0.36 | 0.025 |
| ERBB2 | 1.1355 | 1.7655 | 2.6535 | 3.1105 | 2.1645 | 1.7025 | 2.6215 | 2.6965 | 1.1945 |
| ERP70 | −0.307 | −1.366 | 0.023 | 0.004 | 0.038 | 0.666 | −0.063 | −0.165 | −0.11 |
| ESR1 | −0.2825 | −0.8185 | −0.1295 | −0.4845 | −0.5425 | −1.1105 | −1.3435 | −0.8425 | −0.4015 |
| ETFA | −0.1125 | 1.1045 | −0.3215 | −0.4765 | −0.4135 | −1.1065 | −1.0505 | 0.3915 | −0.3985 |
| ETV8 | −0.6445 | −0.8885 | 0.0325 | 0.1195 | 0.8275 | 2.0165 | 1.4065 | 0.0465 | −0.1035 |
| EVER1 | 0.2035 | 0.4195 | 0.4575 | 0.4055 | 0.3295 | 0.8955 | 1.2555 | 0.9935 | 0.6145 |
| EXO1 | −0.7675 | −0.0895 | −0.2305 | −0.5325 | 0.3115 | 0.7885 | 0.3805 | 0.4955 | 0.7605 |
| EXOSC9 | −0.453 | 0.264 | 0.261 | 0.036 | 1.028 | 0.771 | −0.026 | 0.541 | 0.205 |
| EXT2 | 0.57 | −0.097 | 0.483 | 0.145 | 0.254 | 1.017 | 1.177 | 0.738 | 0.081 |
| EZH2 | 0.2495 | 0.0165 | −0.0215 | 0.0505 | −0.2045 | −0.1735 | −0.4705 | 0.2035 | −0.2795 |
| FI1R | −0.664 | −0.866 | −1.734 | 1.066 | 0.284 | −0.381 | 0.138 | 0.704 | 1.785 |
| F9 | −1.748 | −0.777 | −1.76 | −0.602 | −0.853 | −1.792 | −1.928 | −0.925 | −0.711 |
| FA2H | 1.08 | 0.74 | 1.667 | 0.032 | 2.532 | 0.776 | 3.02 | 2.675 | 1.65 |
| FABP7 | 0.393 | 1.161 | 0.178 | 1.431 | 0.301 | 1.107 | 0.565 | 0.935 | 0.905 |
| FADD | 0.56 | −0.13 | −0.459 | 0.522 | 0.002 | −1.11 | −0.252 | −0.213 | 0.607 |
| FAHD1 | −1.07 | −0.342 | −0.361 | 0.65 | 0.419 | 0.558 | −0.571 | −0.349 | −0.691 |
| FAHD2A | 0.104 | −0.274 | 0.438 | 0.329 | −0.503 | 0.475 | −0.079 | −0.157 | −0.196 |
| FAM38A | −0.3605 | −0.2035 | 0.7345 | −0.5765 | 0.5545 | −0.4285 | −0.7785 | −0.3925 | −0.1745 |
| FAM3A | −1.1205 | −0.2045 | −0.4735 | 0.3725 | −0.2885 | 0.1365 | −0.5675 | −0.7045 | −0.2965 |
| FAM3B | 0.289 | 0.495 | 0.209 | 0.103 | −0.074 | −0.753 | 0.116 | 0.221 | −0.512 |
| FAM3C | −0.624 | 0.766 | 0.992 | 0.415 | 0.701 | 0.559 | 0.797 | −0.061 | 0.821 |
| FAM46C | −0.172 | −0.57 | 0.426 | 0.103 | 0.173 | 1.355 | 0.729 | −0.099 | 0.814 |
| FAM49B | −0.348 | 0.347 | 0.868 | −0.083 | 0.236 | 0.577 | −0.833 | −0.207 | 0.937 |
| FAM54A | −0.615 | 0.083 | −0.216 | −0.139 | −0.06 | 0.008 | 0.782 | −0.059 | −0.074 |
| FANCA | −0.5825 | −0.4545 | 0.1765 | −0.1815 | 0.0785 | 0.2135 | 0.0285 | −0.0185 | −0.3555 |
| FANCE | 1.5465 | 1.1645 | 1.1775 | 1.1435 | 1.0455 | 0.2245 | 2.0385 | 0.3475 | 1.1245 |
| FBL | −0.236 | 0.571 | 0.008 | 0.352 | 0.034 | 0.316 | −0.104 | 0.368 | 0.13 |
| FBP1 | −0.364 | −0.179 | 0.646 | 0.143 | −0.226 | 0.885 | 0.064 | −0.166 | −0.119 |
| FBS1 | 0.5735 | −0.5685 | −0.6165 | −0.5295 | −0.2035 | 1.8005 | 0.1165 | 0.0995 | 0.4065 |
| FBXL20 | 1.053 | 0.581 | 0.503 | 0.282 | 0.414 | 0.572 | −0.087 | 0.959 | 0.303 |
| FBXO25 | −0.737 | −0.196 | −0.148 | −0.171 | 0.003 | 0.782 | 0.242 | −0.823 | −0.55 |
| FDXR | −0.162 | −0.064 | −1.066 | 0.181 | −0.394 | 1.007 | 2.022 | −0.282 | 3.014 |
| FEM1B | −0.81 | −0.255 | −0.007 | 0.632 | 0.518 | 0.342 | 1.128 | −0.921 | −2.078 |
| FGF12 | −0.7755 | −0.2565 | −0.5205 | −0.0375 | −0.5545 | −0.1595 | −1.0585 | −0.5785 | 0.2225 |
| FGF13 | −1.278 | −0.902 | −0.53 | −1.417 | −0.811 | −0.288 | −1.523 | −0.838 | −0.158 |
| FHOD1 | −0.6705 | −0.8185 | −0.5175 | 0.2275 | −0.0745 | 1.3915 | 3.0465 | 0.3245 | 0.9015 |
| FKBP11 | 0.0735 | 0.1085 | 0.0675 | 0.5535 | 0.0735 | 0.0415 | 0.3805 | 0.5475 | −0.0385 |
| FLJ10094 | −1.417 | −0.559 | −1.567 | −0.54 | 0.02 | −1.305 | −2.022 | −0.453 | −0.657 |
| FLJ10115 | 0.3715 | 0.8405 | 1.6025 | −0.4435 | 0.8055 | 0.6605 | 0.9225 | 0.7695 | −0.1255 |
| FLJ10159 | 0.986 | 1.226 | −0.213 | 0.062 | 0.211 | −1.139 | −1.441 | 0.57 | 0.247 |
| FLJ10204 | 0.712 | −0.916 | 0.702 | 0.181 | 0.254 | 1.304 | 0.901 | 0.512 | 0.258 |
| FLJ10275 | −0.2475 | 3.1575 | −0.1745 | −0.0005 | 1.1085 | 0.5425 | 2.8875 | 2.3875 | 2.2325 |
| FLJ10292 | −0.4535 | −0.1845 | 0.4805 | 0.3095 | −0.1895 | −0.2285 | −0.2895 | 0.0195 | 0.1126 |
| FLJ10324 | −0.4185 | 0.0555 | 0.2125 | 0.5985 | 1.2545 | 1.0865 | 0.5905 | 0.0025 | −0.4075 |
| FLJ10700 | −0.092 | −0.391 | −0.55 | −0.431 | −0.224 | 0.812 | −0.527 | −0.177 | −0.609 |
| FLJ10706 | −0.78 | −0.254 | −0.395 | −0.81 | 0.35 | −0.937 | −1.462 | −1.692 | −0.892 |
| FLJ10671 | 0.862 | −0.039 | 0.988 | −0.794 | −0.092 | 0.866 | 1.576 | 0.475 | 0.607 |
| FLJ10901 | 0.4055 | 1.2445 | 1.5015 | 0.0155 | 1.8765 | 1.1085 | −0.5685 | 1.0085 | −0.1475 |
| FLJ10918 | −1.135 | −0.267 | 0.329 | 0.855 | 1.238 | 0.539 | −0.793 | 0.489 | −0.051 |
| FLJ10980 | 0.1405 | −0.6295 | −0.0765 | 1.0915 | 0.6295 | 1.3945 | 1.1965 | 1.2115 | 0.8475 |
| FLJ11017 | −0.8565 | −0.9985 | −0.7315 | 0.7225 | 0.6065 | 0.9785 | 0.2605 | −0.1985 | −0.2705 |
| FLJ11088 | 0.3815 | 0.4555 | 0.7845 | −0.5585 | 0.4335 | −0.3695 | −0.1825 | −0.3595 | −0.1415 |
| FLJ11151 | 0.7655 | 1.9625 | 0.6525 | 0.8515 | 0.6685 | 0.6185 | 0.6445 | −0.4945 | −0.1735 |
| FLJ11267 | 0.3925 | 0.3415 | 0.7245 | −0.0295 | 0.1835 | 0.4245 | 0.6015 | 0.8835 | 0.3565 |
| FLJ11280 | −0.0255 | −0.1095 | −0.0345 | −0.2975 | −0.3795 | −0.2195 | −0.9455 | −0.3445 | 0.2985 |
| FLJ11508 | −0.4455 | −0.5745 | −1.4095 | 0.3045 | −0.4155 | 0.2235 | 1.9075 | −0.4905 | 0.5245 |
| FLJ12270 | 3.9235 | 3.6595 | 4.0345 | 0.7255 | 2.5025 | 3.3135 | 3.0055 | 4.4225 | 5.1565 |
| FLJ12844 | −0.1085 | 0.1085 | 0.7955 | 0.0035 | −0.0695 | 0.6805 | 0.3565 | 0.4845 | −0.2685 |
| FLJ12650 | 0.7405 | 0.0105 | 0.4085 | 0.1395 | 0.6625 | 0.3065 | 0.4295 | 0.1325 | 0.2025 |
| FLJ12684 | −0.9475 | 0.1475 | −0.5525 | −0.2705 | −0.7175 | −0.9735 | −0.8785 | −0.3885 | −0.2535 |
| FLJ12735 | 1.987 | 0.478 | −0.902 | 1.067 | −0.665 | −0.438 | 0.448 | −0.783 | 0.165 |
| FLJ12750 | 1.238 | 1.636 | 1.406 | 0.404 | −0.078 | 1.177 | 1.139 | 2.554 | 1.41 |
| FLJ12895 | −0.3255 | 0.1165 | 0.3345 | 0.4045 | 0.0845 | 0.3195 | 0.2305 | 0.1695 | 0.2375 |
| FLJ13710 | 0.2595 | −0.6205 | −0.3255 | 0.3645 | 0.5035 | 1.7735 | 0.5805 | −0.0015 | −0.1945 |
| FLJ13855 | 0.165 | 0.033 | −0.483 | −0.189 | 0 | −0.464 | −0.325 | −0.833 | 0.079 |
| FLJ13912 | −0.475 | −0.127 | 1.5 | 1.228 | 0.921 | 0.559 | 0.423 | 1.085 | 1.156 |
| FLJ14124 | −0.363 | 0.277 | −0.638 | 0.063 | −0.423 | −0.503 | 0.556 | 0.58 | 0.49 |
| FLJ14627 | −0.0515 | 0.3775 | −0.3865 | −0.1515 | −0.0175 | −0.0625 | 0.3925 | 0.1795 | 0.3655 |
| FLJ14666 | −1.352 | 0.235 | 1.737 | −0.22 | 0.512 | −1.047 | 2.889 | 0.106 | 0.108 |
| FLJ14904 | 2.448 | 1.016 | 2.85 | 1.598 | 1.244 | 3.743 | 2.788 | 2.849 | 1.806 |
| FLJ20035 | 0.414 | 0.689 | 1.917 | −0.548 | 0.404 | 0.998 | 1.898 | 0.449 | 0.073 |
| FLJ20152 | 0.582 | 0.317 | −0.242 | −0.08 | 0.327 | −0.688 | −0.091 | −0.271 | 0.025 |
| FLJ20171 | 1.448 | 0.429 | 1.103 | 0.504 | 1.018 | −0.083 | −0.109 | −0.412 | −0.58 |
| FLJ20244 | 0.97 | 0.507 | 1.39 | 0.003 | −0.162 | −3.202 | 0.973 | 0.748 | 0.319 |
| FLJ20273 | −0.996 | −0.427 | −0.362 | −0.113 | 0.002 | −0.055 | −0.384 | 0.03 | −0.607 |
| FLJ20315 | −0.195 | −0.298 | 0.284 | 0.14 | 0.583 | 1.539 | 0.538 | 0.353 | −0.055 |
| FLJ20323 | 0.1665 | 0.9185 | 1.1585 | −0.6515 | 0.9685 | 0.1555 | 0.6605 | 0.4255 | 0.5285 |
| FLJ20518 | −0.883 | −0.413 | 0.488 | −0.061 | 0.748 | −0.874 | 0.026 | −0.384 | −0.593 |
| FLJ20530 | 0.191 | 0.216 | 0.049 | −0.01 | −0.434 | 0.309 | −0.602 | −0.251 | 0.377 |
| FLJ20696 | 0.0345 | 1.9445 | 0.9635 | −0.3345 | 0.3685 | 0.3485 | 0.3576 | 0.2425 | −0.4885 |
| FLJ20718 | 0.885 | 1.003 | 1.441 | −0.581 | 0.114 | 0.417 | 0.482 | 0.319 | 0.655 |
| FLJ20772 | 0.4405 | 0.5345 | 0.8425 | 0.6445 | 0.5285 | 0.7055 | −0.1405 | 0.2625 | 0.0585 |
| FLJ20989 | 0.9465 | 0.2245 | −0.5705 | 0.6615 | 0.0795 | −0.6735 | −0.7995 | 0.3585 | −0.1645 |
| FLJ21019 | 2.0885 | 1.3325 | −2.0315 | 1.8725 | 1.0495 | 2.2355 | 1.4995 | 2.5435 | 2.0825 |
| FLJ21062 | 0.2815 | −1.9476 | −0.7895 | −0.1815 | 0.3115 | 0.5285 | −0.2805 | −0.0945 | −1.8625 |
| FLJ21159 | 0.0825 | −0.7435 | −0.1385 | 0.9235 | −0.1785 | 0.5715 | 0.6125 | 1.0295 | −0.1745 |
| FLJ21616 | 0.924 | −1.01 | 1.328 | −0.3 | 0.983 | 2.043 | 0.552 | 1.92 | 0.592 |
| FLJ21827 | −0.6125 | −1.1755 | 1.1115 | −0.2305 | −0.2225 | 0.1525 | 0.8765 | −1.1915 | −0.8125 |
| FLJ21963 | −0.128 | −0.132 | 0.415 | 0.684 | 0.06 | 0.146 | 0.224 | 0.572 | 0.317 |
| FLJ22104 | −1.0285 | −0.5515 | 1.0825 | −0.0475 | 0.4405 | −0.0775 | 0.4395 | −0.2735 | 0.2235 |
| FLJ22222 | −0.926 | 0.366 | −0.131 | 0.004 | −0.01 | −1.638 | 0.405 | −0.017 | −0.401 |
| FLJ22573 | −0.1435 | 1.5995 | 1.0285 | 0.9825 | 0.0725 | −0.6915 | 0.0505 | 1.9145 | 0.1535 |
| FLJ22794 | −1.188 | −0.369 | −0.072 | −0.023 | 0.183 | 1.144 | 0.741 | 3.066 | −0.683 |
| FLJ23188 | −0.8635 | 2.3225 | 0.1885 | −0.1145 | −0.4925 | 1.3505 | 0.2275 | 4.4595 | 0.1135 |
| FLJ23441 | 0.747 | 0.462 | 0.946 | 0.107 | 0.802 | −0.697 | 0.393 | 0.884 | −0.496 |
| FLJ25471 | 0.807 | −0.111 | 1.097 | 0.643 | 0.386 | 1.008 | 0.332 | 0.193 | −0.038 |
| FLJ31204 | −0.7385 | −0.0725 | −0.9705 | 0.2735 | −0.4245 | −0.7525 | −1.7785 | 0.1395 | 0.1125 |
| FLJ31795 | −1.3755 | −0.6555 | 0.4045 | −0.0925 | 0.1265 | 0.1515 | −0.5735 | −0.0495 | 0.6115 |
| FLJ32942 | −0.2705 | −0.8865 | 0.0065 | 2.1475 | 0.7375 | 0.8425 | 2.0715 | 0.8225 | −1.1565 |
| FLJ37970 | −0.222 | −0.116 | 0.451 | 1.579 | 1.427 | −0.023 | −0.118 | −0.095 | −1.313 |
| FLJ39370 | 0.6985 | −0.6325 | 1.0955 | 0.4825 | 0.2665 | −0.0885 | 0.5195 | 0.6435 | 0.0185 |
| FLJ90588 | −1.0125 | 0.2065 | −1.6905 | −0.4175 | −0.3445 | −1.6265 | −3.2075 | −0.1205 | −0.0265 |
| FN3KRP | 1.32 | −0.427 | 0.767 | 0.756 | 0.946 | 1.134 | 1.444 | 0.84 | 0.244 |
| FOLR1 | −0.3595 | −0.8995 | −2.1895 | −1.4495 | −1.3495 | −2.8285 | −2.5125 | −0.0995 | −0.4985 |
| FOXA1 | −0.7245 | −0.0375 | −0.0045 | −0.1155 | 0.6325 | −0.7015 | −1.0295 | 0.3455 | 0.0885 |
| FOXC1 | −0.516 | −0.537 | −1.374 | −0.516 | 0.184 | 0.111 | −0.629 | −1.038 | −0.457 |
| FOXP1 | 0.2885 | −0.5325 | −0.4595 | −0.3385 | −0.5275 | 0.0355 | −0.7475 | 0.0225 | 0.3615 |
| FREQ | 0.1685 | −0.8995 | 0.7265 | 0.4175 | −0.1865 | 0.4095 | −0.2455 | −0.7205 | 0.0385 |
| FTO | −0.2455 | −0.2285 | 0.3465 | 1.0755 | −0.0325 | −0.0955 | −0.1275 | −0.0425 | −0.4125 |
| FUBP1 | 0.0355 | 0.1395 | 1.1775 | 1.4355 | −0.0375 | 1.3535 | 1.1885 | 0.0335 | 0.2175 |
| FUCA1 | −0.414 | −0.478 | −1.205 | 0.19 | 0.678 | −0.338 | −2.215 | −0.576 | −0.003 |
| FUT8 | −0.9935 | −0.5845 | 0.5785 | −0.2525 | 0.1825 | 0.2875 | −0.9155 | −0.0255 | −0.4215 |
| FXYD6 | −1.775 | −0.998 | −1.488 | −0.52 | −0.036 | −0.621 | −0.959 | −0.232 | −0.577 |
| FYCO1 | −2.349 | −1.312 | −1.789 | 0.205 | 0.014 | −0.657 | −0.431 | −0.644 | 2.441 |
| FZD10 | −0.8945 | −0.1825 | −0.5465 | −0.0785 | −0.8485 | 0.1305 | −0.8585 | 0.2215 | −0.1815 |
| FZD7 | −0.7745 | 1.1045 | 2.1075 | −1.2095 | −0.2515 | −1.0535 | 2.6135 | −1.2045 | 0.4065 |
| FZD9 | −0.1205 | 1.2265 | 1.2635 | −0.9295 | 0.1435 | −1.0355 | 1.9855 | −1.2835 | 0.4775 |
| G1P2 | −0.0765 | −0.6335 | 0.0895 | −0.3055 | 0.1125 | 1.3815 | 0.5255 | 0.4975 | 0.4635 |
| G1P3 | 0.018 | −0.084 | 0.024 | −0.13 | −0.177 | 1.113 | 0.599 | 0.003 | 0.951 |
| GA17 | 0.2515 | −0.4365 | 0.2885 | 0.3705 | 1.6055 | 2.3875 | 2.0385 | 1.8865 | 0.7455 |
| GABARAP | 0.0025 | 0.4345 | 0.9205 | 0.9425 | −0.0935 | 0.1295 | −1.4525 | −0.8805 | 0.0335 |
| GAJ | −1.3885 | −0.4225 | −1.7505 | 2.4325 | 1.2885 | −0.5955 | 4.2105 | 1.9795 | 4.1685 |
| GALE | −0.913 | −0.704 | 0.017 | −0.275 | −0.559 | 0.146 | −1.546 | −0.672 | −0.642 |
| GALNT14 | −2.1035 | 0.8385 | −0.8175 | 1.0805 | 0.0105 | −0.0725 | 1.3445 | 0.7885 | 1.5015 |
| GALNT2 | 1.8065 | 0.1255 | 0.6915 | 0.6785 | 0.7635 | 0.4815 | 1.2035 | 1.6485 | 1.2575 |
| GALNT7 | 0.853 | −0.486 | 0.375 | 1.493 | 0.122 | −0.09 | −0.189 | 0.19 | 0.161 |
| GAMT | −0.596 | −0.645 | −0.589 | −0.362 | −0.42 | 0.585 | −0.433 | −0.652 | −0.088 |
| GAPD | −0.707 | 0.05 | −0.028 | −0.454 | 0.141 | 0.54 | 0.006 | 0.336 | −0.235 |
| GARS | 2.316 | 0.605 | 0.557 | 1.382 | 1.258 | 0.649 | 1.441 | 1.321 | 1.008 |
| GART | −0.6855 | −1.7045 | −0.5455 | 1.6075 | 1.6265 | 2.5635 | 1.3845 | 1.8985 | −1.4035 |
| GATA3 | 0.3645 | 0.8215 | 0.7815 | 0.4535 | −0.1005 | 0.3525 | −0.2175 | −0.3555 | −0.3665 |
| GATM | 0.454 | 0.02 | 1.739 | −0.253 | 1.113 | 3.203 | 2.245 | 1.074 | 2.786 |
| GBA | −0.445 | −0.707 | 0.73 | 0.224 | −0.093 | −0.053 | −0.817 | −0.561 | −0.975 |
| GDAP1 | −0.302 | −0.082 | 0.07 | 0.389 | 0.333 | −0.991 | 0.187 | 0.007 | −0.371 |
| GDI2 | 0.4815 | 0.3345 | −0.0755 | 0.5035 | 0.5955 | 1.0155 | 1.3135 | 0.7305 | 0.9965 |
| GGA1 | −1.1515 | −0.0995 | 0.4595 | −1.3075 | 0.7595 | 0.9275 | 0.3195 | 0.8655 | −0.8635 |
| GKAP1 | −1.0245 | −1.0305 | −0.8385 | −0.3095 | −0.5595 | −0.2115 | −1.5335 | −0.5165 | 0.2195 |
| GLCCI1 | −0.3385 | −1.2255 | −0.5635 | −0.1005 | 0.3325 | −1.0625 | 0.0745 | 0.7945 | −0.3475 |
| GLG1 | −0.393 | 0.162 | −0.351 | 0.256 | 0.447 | −1.053 | −0.538 | −0.544 | −0.388 |
| GLTSCR2 | −0.444 | −0.178 | −1.8 | 0.949 | 0.381 | 0.062 | −0.807 | −0.037 | −0.463 |
| GNB4 | 0.293 | 0.372 | 0.149 | −0.153 | 0.062 | −0.02 | 0.521 | 0.247 | 0.581 |
| GOLPH2 | −0.3095 | −0.2835 | −0.5965 | −0.2735 | −0.0435 | −0.3035 | −1.4585 | −0.3145 | −0.3305 |
| GPATC1 | −0.9255 | 0.0035 | −1.3755 | 0.3755 | 1.3055 | 0.8565 | −0.0795 | 0.6175 | −0.8785 |
| GPC2 | −0.0205 | 0.3095 | 0.2155 | 0.6725 | 0.5195 | −0.5815 | 1.7015 | 0.9075 | 0.2215 |
| GPR125 | 0.011 | −0.018 | 0.478 | −1.528 | −0.371 | 0.521 | −0.734 | −0.461 | 1.263 |
| GPR160 | 1.0415 | 0.3285 | −2.8625 | 1.2885 | −0.5105 | 0.4645 | 0.2135 | −0.1015 | 0.2675 |
| GPR56 | 1.1365 | −0.0305 | −0.7785 | −0.0775 | 0.3635 | −0.9085 | 0.0575 | −0.3565 | −0.1435 |
| GPRC5C | −0.3825 | 0.2245 | 0.3885 | 0.5545 | 1.0295 | 0.8935 | 1.2205 | 0.3895 | 0.8305 |
| GPSM1 | −0.255 | −0.957 | 0.43 | −0.544 | 0.195 | 0.251 | 1.161 | −0.538 | −0.261 |
| GPSM2 | 0.413 | −0.625 | −0.763 | −0.425 | −0.507 | 0.738 | −1.022 | 0.343 | 1.019 |
| GPT | −1.5485 | −1.6615 | −1.0565 | −0.7325 | −0.6005 | −0.3535 | −1.8695 | −1.0775 | 1.0075 |
| GPT2 | 0.32 | 3.212 | −1.05 | 4.437 | −0.156 | 0.09 | −1.554 | −1.286 | −0.155 |
| GPX7 | 0.373 | 0.131 | 0.143 | −0.513 | −0.374 | −0.63 | 0.145 | 0.009 | 0.11 |
| GRB14 | 0.4305 | −0.9305 | −0.8505 | −1.0675 | −0.2655 | −1.0485 | −0.4385 | −1.0125 | −0.2485 |
| GRB7 | 0.1315 | 0.2285 | 0.7295 | −0.2745 | −0.1375 | −0.7895 | −1.1745 | −0.3855 | 0.7735 |
| GSC | 0.391 | 0.356 | 0.508 | −0.025 | −0.001 | 0.137 | −0.789 | −0.291 | −0.014 |
| GSDML | −0.329 | −0.065 | 0.082 | −0.308 | 0.026 | 0.438 | −0.18 | −0.165 | −0.164 |
| GSK3B | −0.02 | 0.253 | −0.483 | 0.148 | −0.068 | −0.932 | −0.426 | −0.209 | −0.761 |
| GSTA1 | −0.3775 | 2.2405 | −0.2595 | 2.3475 | 1.8115 | −0.8255 | 0.0205 | 0.2475 | 0.8805 |
| GSTK1 | −2.135 | −2.448 | −1.056 | −1.664 | −1.285 | −1.314 | −3.753 | −2.291 | −1.635 |
| GSTM3 | 1.3705 | 0.1125 | 0.1485 | 0.6915 | 0.1875 | −2.6115 | 0.3115 | −2.0045 | −0.3605 |
| GSTP1 | 0.252 | 0.422 | −0.117 | 0.789 | 0.483 | −1.803 | 2.279 | 2.054 | −0.074 |
| GSTT1 | −0.8065 | −0.7835 | −0.3605 | −0.5385 | −0.3925 | 0.4255 | 0.8405 | −0.0285 | 0.3916 |
| GSTT2 | −0.0465 | 0.5495 | −0.4695 | 0.3625 | 0.4265 | −0.7645 | 0.5245 | 0.9525 | 1.2765 |
| GTF2F2 | −0.842 | 0.424 | −0.006 | −0.651 | −0.514 | 0.012 | 0.61 | 0.178 | 0.01 |
| GTF2I | 0.9065 | 1.2745 | −0.1595 | −0.0735 | 0.4305 | 0.1295 | −0.0595 | 0.5065 | 0.9065 |
| GTF3A | 0.188 | 0.457 | 1.275 | 0.097 | 0.004 | 0.395 | 0.354 | −0.348 | −0.403 |
| GTPBP3 | −0.734 | 0.331 | 1.051 | 0.631 | 0.265 | −1.388 | 1.816 | −0.881 | −0.048 |
| GTPBP4 | −0.429 | 0 | 0.308 | 0.624 | 0.557 | 2.005 | 0.52 | 0.098 | 0.17 |
| H1F0 | 1.4775 | 0.7575 | 0.9825 | −0.0065 | 0.4515 | 0.4245 | −0.1755 | −0.0965 | 0.6105 |
| H2AFV | −0.2775 | −0.0195 | 0.3725 | 1.0985 | 0.7215 | 2.0335 | 1.5805 | 0.8105 | 0.5055 |
| H2AFY | −0.0515 | 0.9785 | 1.0005 | −0.4085 | −0.2505 | 1.2095 | 2.7285 | −1.0565 | 0.3385 |
| H2AFZ | −0.2305 | 0.4035 | 0.8075 | 0.1175 | −0.4385 | 0.5365 | 0.5945 | −0.5585 | −0.4465 |
| H2BFS | 0.1475 | −0.7025 | 0.6816 | −0.5175 | 0.2605 | −0.5645 | −0.0345 | −0.7605 | −0.6405 |
| H3F3B | 1.302 | 1.262 | 0.888 | 0.582 | 0.083 | 0.528 | 0.378 | 1.641 | 1.083 |
| H41 | −0.3105 | −0.1155 | −0.2975 | −0.3905 | 0.0895 | −1.2965 | −1.1915 | −0.1745 | −0.0935 |
| HAGH | −0.822 | −0.628 | 1.331 | 0.879 | −0.323 | 0.945 | 0.631 | 0.046 | 0.439 |
| HBG1 | −0.0005 | 0.9055 | 0.5465 | 0.0785 | 1.6115 | 1.0415 | 0.8435 | 1.1435 | 0.6055 |
| HBL01 | −1 | −0.974 | 0.487 | −0.624 | −0.598 | 0.237 | 0.602 | −0.093 | 0.211 |
| HCAP-G | −1.1125 | −0.6425 | −0.4835 | 0.0425 | −0.4105 | 0.1635 | −0.3595 | 0.0435 | −0.4555 |
| HCP5 | 2.741 | 0.368 | 2.369 | −0.39 | 0.542 | −0.045 | 0.212 | 0.215 | −0.169 |
| HDAC2 | 0.564 | 0.524 | 2.974 | 0.646 | 0.716 | 0.996 | 2.92 | 1.257 | −0.083 |
| HDC | 0.6205 | 0.3825 | 0.0985 | −0.3695 | −0.0385 | −0.3505 | 0.2085 | 0.0305 | 0.3575 |
| HEBP1 | 0.733 | 0.584 | 0.08 | −3.203 | −0.109 | −1.086 | 0.507 | 0.406 | 0.007 |
| HIPK2 | 0.475 | 1.014 | 0.731 | 1.44 | 1.372 | 0.931 | 1.379 | 0.675 | 0.28 |
| HIPK2 | 0.9675 | 1.6255 | 1.1195 | −0.2595 | −0.0805 | 0.9245 | 1.7485 | −0.9475 | 0.4915 |
| HIS1 | 0.7475 | 1.2475 | 0.8525 | −0.3475 | −0.6155 | 0.3565 | 1.9305 | −1.1105 | 0.5735 |
| HIST1H2A | 0.65 | 1.222 | 0.95 | −0.372 | −0.694 | 0.354 | 2.091 | −1.369 | 0.587 |
| HIST1H2B | 0.3975 | 1.4325 | 0.8245 | −0.3955 | −0.5915 | 0.2115 | 2.0045 | −1.2225 | 0.8735 |
| HIST1H2B | 0.633 | 1.456 | 0.916 | −0.279 | −0.439 | 0.328 | 1.98 | −0.921 | 0.701 |
| HIST1H2B | 0.0415 | 0.4025 | 0.6285 | −0.0405 | −0.3935 | 0.3985 | 0.4715 | −0.2385 | −0.8555 |
| HIST1H2B | −0.1515 | 0.4175 | 0.3685 | −0.0915 | −0.3315 | −0.0015 | −0.4965 | −0.0215 | 0.3355 |
| HIST1H3C | −0.8905 | −0.7365 | 1.7925 | 0.1325 | −0.3945 | 1.4765 | −1.1925 | 1.3405 | 0.3195 |
| HM13 | −0.1105 | 0.3755 | 0.8525 | −0.0265 | −0.1245 | 0.7205 | −1.3105 | −0.2345 | 0.3135 |
| HMGB3 | −0.8825 | −1.0555 | 0.2575 | 0.0055 | 0.2695 | 0.2385 | 0.1785 | −0.7285 | −0.0895 |
| HN1 | −0.256 | −0.533 | 0.02 | 0.09 | 0.3 | 0.871 | 0.609 | −0.541 | −0.187 |
| HNRPA3P | 0.8235 | 0.8225 | −1.3885 | −0.2695 | 0.2605 | −0.6685 | −0.9945 | −0.3896 | 0.7505 |
| HNRPDL | 1.453 | 0.078 | −1.612 | −0.404 | 0.574 | −0.99 | −1.049 | −0.432 | −0.51 |
| HOXA5 | 0.4535 | 2.2635 | −2.1855 | 1.6135 | 2.5335 | 1.0255 | −3.8955 | −0.0085 | 2.6115 |
| HOXA7 | −0.7375 | 3.7545 | −1.2795 | 0.4275 | −0.2105 | −0.8895 | −1.7235 | −0.1685 | 1.6375 |
| HOXB2 | 0.3275 | 0.8025 | −1.5965 | −0.6245 | 0.0485 | −0.9615 | −2.5545 | −1.1115 | 0.6375 |
| HOXB6 | 2.0455 | 0.0415 | 1.5295 | −1.1445 | −1.4355 | 0.6565 | 0.5205 | −1.5115 | 1.2925 |
| HOXB7 | 1.2915 | −0.2005 | 1.0595 | −0.2855 | −0.6695 | 2.4435 | 0.2515 | −0.5455 | −0.8035 |
| HOXC10 | 0.069 | 0.245 | 1.859 | 0.173 | 0.03 | −0.873 | 0.107 | −2.34 | 1.021 |
| HOXC13 | −0.8305 | −1.8065 | 0.5235 | −0.1785 | 0.0905 | 0.3245 | 1.0265 | −0.5625 | −0.1305 |
| HOXC9 | −0.5 | −0.304 | 0.361 | −0.085 | 0.34 | −0.033 | 1.156 | 0.428 | −0.251 |
| HOXD3 | 0.4865 | 0.1405 | 0.9825 | 0.0335 | 0.2705 | 0.8585 | 0.7985 | 0.0915 | 0.4885 |
| HPCL2 | −0.894 | −0.477 | −2.788 | −0.231 | 0.065 | −0.882 | 0.132 | −1.65 | 2.951 |
| HPS3 | −0.6555 | −0.4885 | 0.1485 | 0.0545 | −0.0575 | −0.0875 | 0.3525 | −1.2695 | −0.1425 |
| HRASLS | 1.6645 | 1.1215 | 1.2775 | −0.0445 | 0.4345 | 1.4685 | 0.8325 | 0.1425 | 0.0885 |
| HRB | −1.428 | −0.459 | −1.311 | −0.307 | −1.055 | −0.965 | −1.981 | −0.429 | −0.554 |
| HRSP12 | 0.839 | 1.245 | 1.657 | −0.431 | 0.724 | −0.378 | 0.65 | 0.278 | 0.421 |
| HSD17B2 | 0.5885 | 0.4285 | 1.1415 | 0.4195 | 0.2845 | 0.9485 | 0.5795 | 0.1475 | −0.4895 |
| HSF1 | −0.2145 | −0.2025 | 1.7275 | 0.6936 | 0.8285 | 0.4355 | 1.8005 | 0.6455 | −0.3875 |
| HSPA14 | −0.685 | 1.27 | 0.83 | −0.093 | −0.282 | −0.783 | −0.478 | −0.005 | 0.234 |
| HSPA2 | −0.1376 | −0.3335 | 0.4465 | 0.1675 | 0.4145 | 0.7156 | 0.4385 | 0.1635 | 0.4335 |
| HSPA5BP [symbol] | −1.018 | −0.747 | −0.595 | −0.358 | −0.138 | 0.428 | −0.054 | −0.561 | −0.03 |
| HSPC016 | −0.472 | −0.647 | −0.492 | −0.152 | −0.301 | 0.094 | −0.561 | −0.512 | −0.498 |
| HSPC138 | −0.063 | 0.032 | 0.849 | 0.072 | 0.897 | 2.344 | 1.371 | 0.147 | 0.621 |
| HSPC148 | 0.0235 | 0.7065 | 0.5635 | −0.2225 | 0.5345 | 1.3265 | 0.2875 | −0.6985 | −0.6365 |
| HSPC150 | −0.1895 | −0.0775 | 0.3125 | 0.3135 | −0.1985 | −0.1955 | 0.8745 | 1.0855 | 0.9875 |
| HSPC163 | −0.748 | 0.203 | 0.64 | 0.223 | 0.607 | 1.913 | 1.091 | 1.484 | 3.298 |
| HSPC266 | 0.037 | −1.132 | 0.52 | −0.588 | 0.117 | −0.816 | 0.956 | −1.402 | −0.294 |
| HSU79274 | −0.424 | 0.394 | 0.198 | 0.051 | 0.054 | 0.08 | −0.322 | −0.417 | 0.49 |
| HTATIP2 | 0.489 | 0.904 | 0.957 | 8.076 | −0.272 | 0.752 | 0.192 | 0.273 | 0.54 |
| ICMT | −0.635 | −0.055 | −1.789 | 1.549 | −0.372 | −2.278 | −2.774 | −1.649 | −0.426 |
| ICT1 | −1.099 | −0.069 | 1.338 | 0.106 | −0.371 | 0.975 | −1.125 | −0.548 | −0.628 |
| ID4 | 0.4725 | −0.2895 | 0.3385 | 0.4465 | 0.1825 | −0.0495 | −0.2865 | 0.4075 | −0.5835 |
| IDH2 | −1.484 | −0.509 | 0.675 | −0.604 | −0.264 | −0.577 | −0.26 | −0.332 | −0.104 |
| IDS | −1.1945 | 8.0455 | 1.1095 | −0.8065 | −0.0015 | −0.9115 | 1.9655 | −1.7365 | −0.7945 |
| IFI30 | −0.6545 | 0.4825 | 0.9455 | −0.0475 | 0.1525 | −0.0185 | 2.1105 | −0.0055 | 0.3085 |
| IFI44 | −1.3175 | −1.1625 | 1.7815 | −0.8545 | 1.0145 | −0.3505 | 1.9535 | 0.1245 | −0.1835 |
| IFIT5 | 0.1245 | −0.1485 | −0.3945 | 0.8595 | 0.0016 | −0.0945 | −0.0215 | 0.5445 | 0.0745 |
| IFITM1 | 1.0685 | −0.1965 | 0.8305 | 2.5995 | 0.7095 | 2.2685 | 1.9385 | 1.0645 | 1.2785 |
| IGBP1 | 8.3165 | 2.1915 | 1.5335 | 1.7635 | 1.3545 | 0.2085 | −1.1415 | −0.1195 | 0.2565 |
| IGFALS | −0.0525 | −0.7435 | 1.4765 | −0.6505 | −0.3195 | −0.7615 | −0.5895 | −0.8855 | −0.9845 |
| IGPBP2 | 0.3485 | −0.4105 | 1.4855 | −0.5005 | −0.1855 | −0.4845 | −0.4065 | −0.3695 | −0.1055 |
| IGHG1 | −2.9765 | −1.1475 | 0.1035 | −3.1675 | −0.2085 | −2.2265 | −4.3055 | −3.3945 | −1.9495 |
| IGHG1 | −0.913 | −1.362 | 2.57 | −1.536 | −0.823 | −1.354 | −1.139 | −1.55 | −1.471 |
| IGJ | −1.085 | −2.59 | 1.521 | −2.415 | −1.454 | −2.549 | −2.824 | −2.671 | −1.503 |
| IGKC | −0.189 | −0.623 | 1.023 | −0.83 | −0.572 | −0.609 | −0.506 | −0.649 | −0.749 |
| IGKC | −0.743 | −1.536 | 0.69 | −1.886 | −0.943 | −2.687 | −2.898 | −2.02 | −1.603 |
| IGLC2 | −1.1895 | −0.9155 | 0.0045 | 0.8035 | 0.0895 | 0.8135 | −0.0215 | −0.2895 | −0.1885 |
| IGLL1 | 0.1205 | 0.9535 | −0.3415 | −0.0995 | −0.2875 | −0.1825 | −0.1115 | −0.1045 | −0.4725 |
| IL13RA1 | −0.336 | 0.506 | −0.014 | −0.031 | −0.061 | −0.259 | −0.888 | −0.053 | −0.006 |
| IL17B | 3.5285 | 0.6045 | 1.8845 | 0.7865 | 1.6795 | 0.0375 | 0.3885 | 0.5285 | 0.9035 |
| IL23A | −0.1045 | 0.0865 | −0.0245 | 0.0605 | 0.0565 | 0.4105 | −0.1435 | −0.5646 | −0.2715 |
| IL6ST | −1.1855 | −1.1535 | 0.5185 | −0.2825 | 1.1805 | 1.0705 | −0.4365 | 0.3965 | −0.7195 |
| ILF2 | −1.232 | 0.649 | −0.043 | 1.892 | 0.807 | 1.015 | 0.919 | 0.624 | 0.092 |
| IMPA2 | −0.9725 | −1.2255 | 0.2775 | −1.2805 | −0.4205 | −1.5155 | −1.6675 | −2.1175 | −2.0555 |
| ING1L | 0.869 | −0.61 | 8.002 | 0.799 | 0.664 | −0.002 | 0.584 | 1.064 | −1.049 |
| INHBB | 2.0305 | 3.2245 | 5.1865 | 0.0785 | 0.2175 | 6.0735 | 2.9975 | −0.5645 | 1.4185 |
| INPP4B | −1.6755 | −0.3755 | −1.5635 | −0.7155 | 0.2875 | −1.2775 | −2.2125 | −0.5745 | −0.3555 |
| INSM1 | −1.011 | 0.928 | −0.315 | −0.507 | 0.42 | 8.581 | 0.273 | 0.259 | 0.875 |
| IQCA | −1.263 | −2.326 | 0.609 | 1.43 | 0.928 | −0.743 | 0.826 | −0.003 | 0.488 |
| IQCE | −8.873 | −0.694 | −0.044 | −0.8 | −0.477 | −8.851 | −1.103 | −0.545 | −0.509 |
| IQGAP2 | −0.5135 | −0.4695 | 1.4085 | −1.0515 | −0.4685 | −1.0715 | −1.1985 | −0.8035 | −0.3965 |
| IRF4 | −2.3255 | −0.8005 | −2.0345 | −1.3115 | −0.4075 | −2.8065 | −3.8615 | −1.8565 | −1.5595 |
| IRTA2 | −0.501 | −0.169 | −0.073 | −0.622 | −0.723 | −1.207 | 0.87 | 0.228 | −1.748 |
| IRX4 | −0.4495 | 0.3735 | 1.4465 | −0.4435 | 0.1565 | −0.8185 | 1.2455 | −0.1035 | 0.5825 |
| ISG20 | −0.39 | −0.538 | −2.711 | −0.289 | 0.191 | −3.603 | −1.616 | −0.3 | −0.999 |
| ISGF3 [symbol] | 8.085 | −0.176 | 0.581 | 1.17 | 1.219 | −0.284 | 0.417 | 0.033 | −0.416 |
| ITGB4 | 0.94 | −0.817 | 0.198 | −0.131 | 0.173 | −0.819 | −0.711 | 0.045 | 0.867 |
| ITPR1 | 1.264 | 0.846 | 1.591 | 1.392 | 0.821 | 2.451 | 1.976 | 1.235 | 0.324 |
| JARID1A | −0.508 | −0.607 | −0.544 | −0.084 | −0.107 | −0.336 | 0.001 | −0.233 | −0.476 |
| KARCA1 | 0.057 | 0.022 | 0.038 | −0.552 | −0.309 | −0.193 | −0.002 | 0.002 | 0.067 |
| KATNA1 | −0.9485 | −0.0715 | −0.0695 | −0.6745 | 0.1295 | 0.2585 | −0.0705 | 1.6305 | 0.6325 |
| KCNC2 | 0.433 | 0.137 | −8.095 | −0.663 | −0.963 | −0.687 | −0.447 | −0.051 | 0.088 |
| KCND2 | −0.969 | −0.106 | −1.156 | −0.659 | −0.377 | −1.517 | −0.87 | −1.391 | −0.906 |
| KCNK4 | −1.064 | −1.77 | −0.516 | 1.27 | 1.109 | 1.548 | 0.743 | −0.271 | −0.963 |
| KCNN4 | 0.2865 | 0.0745 | −0.9835 | −0.2125 | 0.3755 | −0.5405 | −0.7665 | 0.0615 | 0.5265 |
| KCNS3 | −0.3745 | 0.9095 | 1.1435 | 0.8245 | 1.0075 | 1.1875 | 1.4085 | 0.9845 | −0.3525 |
| KCTD15 | −1.674 | −0.363 | 0.014 | 0.579 | −0.22 | 1.264 | −1.105 | −0.069 | −0.856 |
| KCTD3 | −0.507 | −0.942 | −0.208 | −0.141 | 0.101 | −0.283 | −0.052 | −0.853 | −0.755 |
| KDELR2 | 1.36 | 1.01 | 1.119 | 0.397 | 1.973 | 3.163 | 1.454 | 0.91 | 0.893 |
| KIAA0020 | 0.226 | −0.509 | 1.536 | −0.293 | −0.025 | 0.126 | −0.445 | 1.898 | −0.763 |
| KIAA0101 | 0.2415 | 0.8485 | 0.5245 | −0.2215 | −0.4285 | 0.5735 | −0.5325 | 0.6445 | 1.4405 |
| KIAA0153 | −0.2945 | 1.6105 | 2.1195 | −0.7555 | 0.3115 | 8.3855 | 0.8745 | 0.1015 | 0.0355 |
| KIAA0182 | −0.9335 | 0.0035 | −0.0145 | 0.4255 | −0.2245 | 0.4155 | −0.2315 | −0.4485 | −0.4485 |
| KIAA0196 | −0.2575 | 0.6285 | 0.2805 | 0.4085 | 0.2535 | 0.8255 | −0.1325 | 0.5835 | 0.3775 |
| KIAA0241 | −0.267 | −0.553 | 0.365 | 0.465 | 0.213 | −0.146 | −0.209 | −0.364 | −0.175 |
| KIAA0251 | 0.0165 | 0.3135 | −0.3705 | −0.1855 | 0.2555 | −0.7765 | 0.1505 | −0.3635 | 0.0095 |
| KIAA0274 | 1.427 | 0.232 | −0.152 | −0.258 | 0.481 | −0.327 | 0.317 | 1.096 | 0.148 |
| KIAA0515 | −0.4355 | −1.1705 | −0.0405 | −0.7555 | −0.0335 | −0.3015 | 0.6595 | −0.3675 | −0.2955 |
| KIAA0582 | −0.4305 | 8.0135 | −0.5475 | −0.2005 | −0.2105 | −0.5675 | 0.2215 | 1.0015 | 1.6025 |
| KIAA0650 | −0.9145 | −0.8055 | −0.5135 | −0.1735 | 0.0365 | −0.0215 | −0.6465 | −0.5045 | 0.4495 |
| KIAA0703 | 0.452 | 0.366 | −0.002 | −0.289 | 0.722 | −0.823 | −1.958 | 0.1 | 0.588 |
| KIAA0830 | −0.7185 | 1.0105 | −0.2075 | 0.0495 | −0.2795 | −1.3535 | 0.1875 | 0.1705 | −0.1955 |
| KIAA0980 | −1.0365 | 0.3685 | −0.3775 | 0.0315 | −0.3135 | 0.2965 | 0.1175 | 0.0775 | 0.2185 |
| KIAA1324 | 0.781 | −0.253 | −0.828 | −0.598 | −0.804 | −0.564 | −0.151 | 0.075 | 1.48 |
| KIAA1363 | −0.337 | −0.257 | −1.061 | 0.147 | −0.506 | −0.193 | −0.102 | −0.266 | −0.986 |
| KIAA1683 | 0.1045 | 0.0835 | 0.1185 | −0.2765 | 0.9555 | −0.4945 | −0.1615 | −0.1425 | 0.4515 |
| KIF12B | 0.2745 | −0.1505 | 0.4345 | 0.1085 | −0.2165 | 0.1615 | 0.3715 | −0.1845 | −0.7025 |
| KIF24 | −0.0555 | −0.0015 | −0.3295 | −0.0745 | −0.3575 | −0.3975 | −0.2485 | −0.2635 | 0.1715 |
| KIN | −2.4075 | −2.0065 | 0.4165 | −1.2855 | −0.1535 | −1.0855 | −2.7545 | −0.7075 | −0.8405 |
| KIRREL3 | −0.0445 | 0.4435 | −0.2975 | −0.7175 | −0.6465 | −0.6855 | −0.3895 | −0.7705 | −0.3855 |
| KIT | −0.9445 | 0.2515 | −2.2485 | −1.4095 | −1.0005 | −2.6175 | −2.3805 | −1.6955 | −0.7685 |
| KLK5 | 0.169 | −0.299 | −0.558 | −0.533 | −0.425 | −0.803 | −0.44 | −0.724 | −0.444 |
| KLK6 | −0.611 | −0.006 | −1.724 | −0.735 | −0.309 | −1.258 | −1.84 | −0.62 | −0.165 |
| KLK7 | −0.185 | 0.381 | −0.515 | 0.119 | 1.225 | 1.585 | 1.12 | 0.636 | 0.933 |
| KLK8 | 0.4975 | 0.4785 | 0.6275 | 0.0975 | 0.9565 | 0.9575 | −0.0755 | −0.3225 | 0.1995 |
| KNTC2 | −0.679 | −0.497 | −1.017 | −0.156 | −0.601 | 0.219 | 2.717 | 0.195 | 0.303 |
| KPNA2 | 0.2165 | 0.1875 | 1.0625 | 1.1195 | 0.2905 | 1.4425 | 1.0216 | 0.7765 | 0.2615 |
| KREMEN2 | −1.1315 | −0.2655 | 2.7455 | −0.8285 | −0.6405 | 0.0395 | −1.1565 | 0.8015 | −1.3965 |
| KRT10 | −1.2045 | −0.5645 | 2.7225 | −1.1775 | −0.8965 | 0.2245 | −1.7735 | 0.8815 | −1.7295 |
| KRT13 | 0.178 | 0.529 | −1.422 | −1.313 | −0.251 | −1.713 | −2.486 | −2.158 | −0.902 |
| KRT15 | 0.552 | 0.986 | 1.537 | 0.946 | 0.098 | −0.756 | 0.651 | 1.372 | 0.767 |
| KRT17 | −2.9276 | 0.2795 | −1.7685 | 0.3705 | 0.1255 | 0.0525 | −5.1155 | −0.4815 | −1.6105 |
| KRT18 | −1.332 | −0.029 | −0.991 | −2.063 | −1.129 | −2.266 | −2.576 | −2.412 | −0.944 |
| KRT23 | 0.3905 | 0.1805 | −0.1125 | 0.0385 | −0.2125 | 0.2595 | 0.3095 | −0.2095 | 0.1295 |
| KRT6B | 0.695 | 1.581 | 2.918 | 1.221 | 0.482 | 1.3 | 0.26 | 1.966 | 0.09 |
| KRTAP3-3 | 0.0845 | 2.7165 | 1.9905 | −1.0635 | 1.8775 | 0.2345 | 0.4125 | 1.5165 | 0.7275 |
| KRTHA7 | −1.0855 | −0.4655 | −2.2555 | 0.0645 | −0.2855 | −0.8865 | −2.8215 | −2.1955 | −2.2045 |
| LAMA3 | 1.118 | 0.925 | 0.797 | 0.654 | 0.236 | 0.352 | 0.169 | 0.193 | −0.394 |
| LAMB3 | −0.39 | −0.104 | 0.99 | −0.593 | −0.242 | −0.436 | −0.343 | −0.339 | −0.104 |
| LASS2 | −0.8785 | −1.0035 | −0.5365 | −0.5785 | −0.3595 | −0.4095 | −0.4325 | −1.4235 | −0.5295 |
| LAX | −1.0945 | −1.9345 | 1.0595 | −0.8365 | 0.3935 | 0.5895 | 3.0715 | −1.3135 | −0.8265 |
| LBR | 0.1265 | −1.4405 | −0.2005 | −1.1065 | 0.3935 | 0.1515 | 1.0655 | −1.9205 | −1.1875 |
| LCE1B | −1.1975 | −0.6645 | −1.0105 | 0.3425 | 0.9905 | 0.4055 | 1.2075 | −0.1865 | −0.1225 |
| LDHB | −0.26 | 0.124 | 0.094 | −0.156 | 0.054 | −0.615 | 0.147 | 0.322 | 0.412 |
| LDOC1L | 0.675 | −0.22 | −0.373 | −0.824 | 0.17 | 0.402 | 1.876 | 1.33 | 1.19 |
| LETMD1 | −1.7925 | −0.2805 | 0.9525 | −1.9405 | −1.4735 | −1.0895 | −1.5335 | −0.7075 | −1.1625 |
| LFNG | 0.193 | −0.211 | −0.194 | 0.031 | −0.051 | 0.417 | 0.371 | 0.102 | −0.76 |
| LGALS2 | 0.489 | 0.021 | −0.231 | −0.344 | 0.314 | 0.079 | 1.192 | 1.685 | 1.129 |
| LGTN | 1.794 | 0.933 | 1.671 | 1.78 | 1.315 | 1.146 | 1.14 | 1.283 | 1.326 |
| LNX | 0.3655 | −0.4165 | 1.4775 | −1.3395 | 0.7185 | 1.1675 | 1.3435 | 0.6245 | −0.2265 |
| LOC11247 | −0.31 | −0.06 | −0.966 | −0.206 | 0.237 | −0.263 | 0.956 | −0.978 | −0.476 |
| LOC11492 | 0.645 | 0.359 | 0.595 | 0.02 | 0.388 | 2.05 | 0.821 | 0.466 | 0.874 |
| LOC11497 | −0.7245 | −0.4035 | −0.1465 | −0.5695 | −0.5935 | −0.0245 | −0.0865 | −0.5075 | −0.3465 |
| LOC11621 | −0.1985 | −0.5945 | 0.5315 | 1.5765 | −0.8125 | −0.8895 | 0.1505 | 0.9865 | −1.0755 |
| LOC11644 | −0.1965 | −1.0335 | 0.2425 | 0.7915 | −0.5035 | −0.0395 | 0.0075 | 0.2475 | −0.2095 |
| LOC12022 | 0.1545 | 0.3765 | 0.3475 | 0.0815 | −0.1665 | 0.9735 | 0.7305 | 0.6695 | 0.5305 |
| LOC12422 | 0.0915 | 0.6705 | −0.1455 | −0.3776 | −0.0385 | −0.8735 | −0.5785 | −1.0215 | 0.5195 |
| LOC14654 | 0.485 | 1.307 | 0.061 | 0.819 | 0.447 | 0.133 | 0.141 | 1.465 | −0.87 |
| LOC15506 | −0.626 | 0.524 | −1.003 | 0.592 | 0.486 | 0.732 | 3.02 | 1.735 | 2.459 |
| LOC20189 | −1.8375 | −1.4445 | −1.2465 | −0.9195 | −0.4925 | 4.3455 | 0.1335 | −0.5565 | 3.2645 |
| LOC25398 | −0.514 | 0.093 | −0.407 | 0.092 | −0.496 | −0.778 | −0.654 | 0.002 | 0.828 |
| LOC33990 | 0.949 | 0.906 | 0.501 | −0.585 | −0.231 | −0.314 | 1.232 | 1.229 | 0.698 |
| LOC34817 | 1.2025 | 0.7105 | 0.6195 | −1.2915 | −0.3925 | −0.3385 | 0.0875 | −0.4045 | −0.4825 |
| LOC40045 | −2.177 | −1.713 | −1.073 | 0.745 | 0.023 | 0.16 | −1.97 | −0.825 | −0.43 |
| LOC44089 | 0.791 | 0.371 | 1.835 | −0.576 | −0.073 | 0.453 | 0.646 | 0.136 | 0.114 |
| LOC49386 | −1.953 | 0.391 | 0.705 | 0.504 | 1.162 | 0.623 | 1.599 | 0.945 | 0.907 |
| LOC51123 | 1.232 | 0.243 | 0.502 | 0.841 | 0.197 | 0.799 | 0.36 | 0.36 | −0.208 |
| LOC51161 | 1.7995 | −0.0275 | 1.8905 | 1.3155 | 0.9755 | 1.5085 | 1.1135 | −0.2815 | 0.3705 |
| LOC51321 | 0.7265 | 0.4805 | −0.6785 | 0.3315 | −0.3965 | −0.9795 | −1.3525 | −0.0125 | −0.3195 |
| LOC51760 | −0.7755 | 0.3785 | −1.0595 | 0.2595 | −0.3025 | 1.0185 | −0.8975 | −0.4835 | 0.2335 |
| LOC81558 | 1.826 | 1.073 | 1.644 | 0.67 | 0.978 | 1.167 | 1.363 | 0.762 | −0.14 |
| LOC81569 | −0.9825 | 0.8495 | 0.4825 | −0.4555 | 0.0885 | 0.9835 | 0.8045 | −0.1975 | 0.8975 |
| LOC90355 | −0.106 | 1.228 | 0.851 | 0.245 | 0.167 | 0.555 | −0.52 | −0.55 | −0.107 |
| LOC90701 | −2.375 | −0.463 | 1.1 | −1.901 | 0.517 | −2.956 | 0.527 | −1.955 | −1.753 |
| LONP | 0.2355 | 0.0825 | 1.4055 | 0.5615 | 0.3885 | 0.5465 | 0.3525 | 0.8875 | −0.0635 |
| LRAP | −0.2145 | 0.0035 | 1.2225 | −0.2985 | 1.1905 | −0.1235 | −0.5725 | 1.0795 | 0.4835 |
| LRBA | 1.2525 | −0.4415 | −0.1245 | 0.7185 | −0.0025 | −0.2165 | 1.1935 | 0.3515 | 0.5535 |
| LRIG1 | −0.1235 | 1.0095 | −0.5345 | −0.7005 | −0.2245 | 0.0195 | −0.0995 | −0.6005 | 0.2665 |
| LRP6 | −1.445 | −0.764 | −0.882 | 0.432 | 0.651 | −0.38 | −1.08 | 0.417 | −0.639 |
| LRP8 | 1.333 | 0.959 | 1.104 | 0.206 | 0.056 | 0.167 | 0.079 | −0.135 | 0.518 |
| LRRC17 | 0.0625 | −0.0585 | 1.3105 | −0.9276 | −0.6965 | 1.1005 | 2.0545 | 2.5175 | 2.8475 |
| LRRC2 | 0.0125 | −0.1905 | −0.1575 | 0.1565 | 0.2295 | 0.8855 | 0.4345 | −0.5645 | 0.3035 |
| LSM1 | −0.9425 | −1.2885 | 1.3425 | −1.9255 | −0.9995 | −1.5615 | 0.7095 | −1.9695 | −0.9895 |
| LSM8 | −0.275 | −0.139 | −0.211 | −0.141 | −0.305 | 0.009 | −0.103 | −0.079 | −0.248 |
| LTB | −0.821 | −0.894 | 0.351 | −0.424 | 0.283 | −0.9 | −0.951 | −0.9 | −0.224 |
| LY6D | 0.425 | −0.165 | 0.755 | 0.825 | 1.215 | 2.091 | 1.751 | 1.165 | 0.4 |
| LYN | 0.671 | 0.578 | 0.435 | 0.892 | 0.289 | 0.804 | 1.136 | 1.087 | 0.973 |
| MA02L1 | −0.016 | −0.264 | −0.578 | 0.897 | −0.143 | −0.531 | −0.403 | −0.224 | 0.013 |
| MADP-1 | −0.9295 | −0.4805 | −0.9825 | −0.1345 | 0.0645 | −1.2065 | −1.6845 | 0.0965 | −0.5845 |
| MAGEA12 | −0.238 | −0.118 | −0.225 | 0.092 | −0.052 | −0.34 | −0.43 | −0.085 | −0.02 |
| MAGEA2 | −0.6625 | −0.6505 | −0.4505 | 0.0595 | 1.1105 | −0.9376 | −1.9415 | 0.2455 | 0.6645 |
| MAGEA3 | −1.3315 | −0.5245 | −1.5015 | −0.2905 | 0.5885 | −1.5885 | −1.1805 | −1.3495 | −0.8835 |
| MAGEA4 | −0.292 | 0.418 | −1.491 | −1.059 | −0.474 | 1.512 | −0.914 | 1.286 | −1.502 |
| MAGED4 | −0.4185 | −0.0485 | 1.7285 | −0.3065 | 0.4355 | −0.0025 | −0.1415 | 0.1225 | 0.1365 |
| MAGI1 | 0.9795 | 0.6245 | 1.5685 | −0.4475 | 1.1205 | 0.4965 | 1.6735 | −0.2325 | −1.0375 |
| MAGI-3 | −1.022 | 0.402 | −0.37 | −0.016 | 0.389 | −0.422 | −0.804 | −0.165 | −0.185 |
| MAL2 | 1.134 | 1.081 | 0.873 | −0.513 | 1.349 | 0.259 | 2.237 | 0.565 | 0.729 |
| MAN2B1 | −0.0275 | −0.7885 | −0.0395 | −0.3815 | 0.1335 | 0.1835 | −0.6255 | −0.6455 | −1.1245 |
| MAPK8IP2 | −1.7745 | −1.0215 | 0.3665 | −0.8375 | −0.1405 | 0.5855 | −1.0745 | −1.4275 | 3.2925 |
| MARCO | −0.055 | −0.365 | 1.796 | 0.772 | −0.08 | −0.279 | −1.926 | −0.012 | −1.141 |
| MARLIN1 | 0.011 | −0.285 | 0.533 | −0.172 | 0.809 | 0.869 | 1.184 | 0.316 | 0.11 |
| MASS1 | −0.3355 | −0.0015 | −0.2865 | 0.0335 | 0.0295 | 0.7075 | 1.1095 | 0.3935 | 0.8525 |
| MCM2 | 1.9235 | 0.1445 | 0.7115 | 0.7205 | 0.0735 | 1.7525 | −0.4595 | 0.2515 | −0.4945 |
| MCM3 | 0.108 | 0.729 | 0.788 | 0.68 | −0.25 | 1.272 | 1.057 | 0.141 | 0.378 |
| MESP1 | −0.0925 | −1.5415 | −0.8615 | −0.0945 | 0.4325 | −1.4005 | −2.5385 | −0.0205 | −0.6845 |
| METRN | −0.3745 | 0.2865 | −0.8385 | −0.3305 | 0.0295 | −0.7295 | −0.5135 | 1.0385 | −0.3025 |
| MFAP2 | 1.184 | 0.321 | 0.145 | 0.153 | −0.115 | −0.094 | 0.081 | −0.187 | −0.069 |
| MFGE8 | −0.2945 | 0.7415 | −0.2245 | −0.0205 | 0.0975 | 0.5035 | −0.0725 | 0.0275 | 0.0755 |
| MGAT4B | −0.172 | −0.48 | 0.155 | −0.368 | −0.312 | −0.15 | −0.96 | −0.617 | −0.402 |
| MGC1091 [symbol] | 0.2505 | 0.6405 | 1.9185 | 0.5145 | −0.3635 | 0.6245 | 0.0695 | 1.1975 | 0.2505 |
| MGC1098 [symbol] | −0.2555 | 0.4935 | 0.2755 | 0.7755 | 0.5805 | 0.9795 | 0.0305 | −0.2515 | 0.2735 |
| MGC1124 [symbol] | −0.839 | −0.427 | −0.515 | −0.133 | −0.037 | −0.189 | 0.237 | −0.187 | −0.058 |
| MGC1127 [symbol] | 0.12 | −0.572 | 0.299 | 0.049 | 0.412 | −0.311 | −0.5 | 0.595 | 0.936 |
| MGC1312 [symbol] | 0.237 | 0.54 | 0.555 | 0.402 | 0.05 | 0.155 | 0.365 | 0.082 | 0.305 |
| MGC1318 [symbol] | 0.341 | −0.293 | 0.3 | 0.338 | −0.081 | 0.501 | 0.508 | 0.016 | 1.004 |
| MGC1414 [symbol] | −0.2705 | −0.3415 | −0.0745 | 0.2425 | −0.0555 | 0.2085 | −0.1045 | −0.3575 | 0.4525 |
| MGG1428 [symbol] | −0.308 | 0.339 | 0.81 | 1.444 | 0.134 | 0.96 | 1.394 | −0.022 | −0.178 |
| MGC1481 [symbol] | −0.352 | −0.218 | 1.375 | −0.426 | −0.406 | −0.553 | 0.246 | −0.563 | 0.015 |
| MGC1580 [symbol] | −0.3205 | −0.3555 | −0.2965 | −0.5015 | −0.1165 | 0.6085 | 0.6045 | 3.3165 | −0.6415 |
| MGC1663 [symbol] | −0.275 | 0.028 | −0.294 | 0.162 | 0.495 | 0.651 | 0.423 | 0.411 | 0.076 |
| MGC1873 [symbol] | 0.704 | −0.472 | −0.051 | 0.363 | 0.01 | −0.042 | −0.905 | −0.04 | 0.464 |
| MGC2053 [symbol] | −0.024 | −0.135 | 0.231 | 1.546 | 1.086 | 0.852 | 2.156 | −0.162 | 0.558 |
| MGC2398 [symbol] | −1.474 | −0.562 | −0.382 | −0.11 | −0.359 | 0.691 | −0.832 | −0.658 | −0.368 |
| MGC2610 | −0.339 | −1.435 | 1.128 | −1.08 | −1.158 | −1.335 | −1.143 | −1.296 | −1.237 |
| MGC2714 | −0.044 | −0.656 | 1.423 | −0.945 | −0.762 | −0.872 | −0.743 | −0.6 | −0.792 |
| MGC2716 [symbol] | −0.2485 | −0.7395 | 0.9335 | −0.9555 | −0.7155 | −0.9865 | −0.9275 | −0.8585 | −0.8995 |
| MGC2716 [symbol] | −0.9085 | −0.9805 | 1.6315 | −1.2765 | −0.9475 | −1.0845 | −1.0645 | −1.1525 | −0.8085 |
| MGC2716 [symbol] | −0.734 | −1.057 | 1.073 | −1.242 | −0.903 | −0.889 | −1.057 | −0.983 | −0.851 |
| MGC2716 [symbol] | −0.2595 | 0.0995 | −0.0025 | 0.1045 | 0.1235 | 1.1655 | 0.6775 | −0.3345 | 0.2025 |
| MGC2716 [symbol] | 0.194 | 0.273 | 1.35 | 2.09 | −0.321 | 0.510 | 0.884 | 2.027 | 1.069 |
| MGC2887 [symbol] | −0.011 | 0.357 | 1.505 | 0.476 | 0.973 | 0.981 | −0.42 | 0.143 | 0.201 |
| MGC3212 [symbol] | −0.655 | −0.473 | −0.232 | −1.523 | −0.253 | −0.946 | −1.696 | −1.411 | 0.224 |
| MGC3321 [symbol] | −0.1935 | −0.4425 | 0.4645 | 0.2705 | −0.3175 | 0.5585 | −1.2865 | −0.8925 | −1.4845 |
| MGC3484 [symbol] | 1.1175 | 1.0345 | 0.7325 | −1.3985 | 0.7685 | −0.2455 | 0.5895 | −0.3445 | 0.8295 |
| MGC3492 [symbol] | 3.417 | 2.101 | 2.199 | 0.772 | 1.721 | −1.654 | 0.771 | 2.719 | 0.243 |
| MGC4021 [symbol] | −0.051 | −0.465 | 0.44 | 0.03 | 0.684 | 1.277 | 0.802 | 0.362 | 0.44 |
| MGC4251 | 0.6685 | 0.8515 | 0.0645 | 1.2445 | 0.4685 | 0.9535 | 1.0405 | 1.0055 | 0.0485 |
| MGC4308 | 0.592 | 0.078 | 1.625 | 1.71 | 0.532 | 1.584 | 2.196 | 0.725 | 0.922 |
| MGC4800 | −1.0555 | 0.4515 | −2.2105 | −2.1945 | −0.7935 | −2.2755 | −2.7955 | −1.9405 | −0.6555 |
| MGC4659 | −1.265 | −0.599 | −1.397 | −1.297 | −0.238 | −1.137 | −1.65 | −0.951 | −0.754 |
| MIA | −0.9945 | −0.2145 | −1.5195 | −0.8155 | −0.0515 | −1.3105 | −1.9255 | −1.0555 | −0.6295 |
| MID1 | −1.9435 | −1.2055 | 2.3716 | −1.8085 | −0.1585 | −1.3165 | −2.1635 | −1.2855 | −1.1595 |
| MID1 | −0.37 | 0.315 | 0.747 | 0.755 | 1.646 | 1.627 | 1.51 | 0.993 | 0.226 |
| MID1 | 0.2875 | −0.5075 | 1.0765 | 0.9355 | 0.6675 | 0.5495 | 1.0215 | −0.1975 | −0.7755 |
| MLF1IP | −1.8865 | −1.2545 | 0.1205 | 0.4295 | −1.3345 | 0.5435 | −1.1335 | −0.9055 | 0.9405 |
| MLSTD1 | −2.479 | −2.096 | −0.107 | −2.698 | 0.442 | 0.116 | −0.626 | −2.806 | −0.037 |
| MMP1 | 0.38 | −0.599 | 1.759 | 0.755 | −0.123 | 1.102 | 1.005 | 0.49 | 0.689 |
| MMP7 | 0.3145 | 0.0855 | −0.0355 | 0.2695 | −0.9415 | 0.1315 | 0.1815 | 0.0775 | −0.2235 |
| MNAT1 | −1.0855 | 0.1935 | 1.5935 | −0.2585 | 0.8445 | 0.5485 | −0.3005 | 0.1025 | −0.4545 |
| MRPL14 | −0.031 | 0.203 | 0.701 | 0.488 | −0.048 | −0.018 | −0.159 | 0.417 | 0.282 |
| MRPL15 | 0.6215 | 0.4655 | −0.6645 | −0.0275 | −0.2145 | −0.2085 | 0.3685 | 0.3365 | 0.0805 |
| MRPL27 | 0.3585 | 0.7035 | 0.5085 | 1.4415 | 0.4325 | 1.2345 | 0.4985 | 1.1015 | −0.1385 |
| MRPL34 | 0.223 | 0.281 | 0.771 | 0.552 | 0.323 | 0.475 | 0.385 | 0.354 | 0.307 |
| MRPL43 | −0.7885 | −0.7785 | −0.2135 | −0.3285 | 2.4005 | 0.1015 | 0.3485 | −0.4885 | 0.8415 |
| MRPL45 | −0.012 | −0.085 | −0.236 | −0.043 | −0.012 | 0.54 | 0.596 | −0.66 | −0.09 |
| MRPL48 | −0.4375 | −0.1705 | 0.3375 | 0.7395 | 0.4875 | 1.3735 | 0.8815 | −0.2765 | −0.3475 |
| MRPL9 | 0.2505 | 0.9155 | 0.3455 | 0.3665 | 0.0595 | −0.1935 | −0.3495 | 0.1505 | 0.3095 |
| MRPS14 | 0.409 | 0.4 | 0.012 | −0.162 | 0.423 | 1.32 | 0.439 | −0.227 | 0.256 |
| MRPS15 | −0.006 | −0.03 | 0.077 | 0.229 | 0.102 | 1.039 | 0.712 | 0.161 | 0.208 |
| MRPS17 | −0.371 | 0.349 | 0.175 | 0.236 | −0.027 | 0.69 | 0.134 | −0.057 | 0.397 |
| MRPS24 | −0.6615 | −0.8005 | −0.4335 | −0.9385 | −0.2925 | −0.2865 | 0.7635 | −0.0495 | 0.4545 |
| MRPS25 | −0.238 | −0.839 | −0.595 | −0.043 | 0.445 | 0.949 | 0.174 | 0.606 | −0.667 |
| MRPS31 | 0.435 | −0.009 | 0.599 | −0.41 | −0.075 | −0.301 | −0.479 | −0.298 | −0.347 |
| MRPS6 | −0.4085 | −0.0955 | 0.2195 | −0.1145 | 0.6865 | 0.2575 | 0.7645 | 0.2905 | 0.6555 |
| MS4A1 | 0.7655 | −0.4015 | 0.2925 | 0.2195 | −0.0005 | 0.9565 | 0.2275 | 0.5815 | 0.4275 |
| MSH2 | −0.283 | 0.394 | 4.448 | −0.646 | −0.131 | −0.469 | 0.469 | 0.386 | 0.428 |
| MSH3 | −0.677 | −0.196 | −0.025 | −0.273 | −0.498 | −0.608 | −0.97 | −0.666 | 0.159 |
| MSMB | −0.206 | 0.864 | 0.021 | 0.474 | 0.451 | −0.043 | 0.031 | 0.216 | 0.314 |
| MTA1 | −1.1945 | −0.8835 | 1.5775 | 0.1335 | −0.7445 | 0.6935 | 1.6875 | −0.4825 | −1.2875 |
| MTA3 | 0.215 | 0.086 | −0.162 | 0.136 | −0.698 | −0.766 | 0.357 | −0.193 | 0.196 |
| MTAC2D1 | −0.071 | −0.705 | 1.009 | 0.456 | −0.088 | 0.589 | 0.55 | 0.45 | 0.612 |
| MTCH1 | −0.9525 | −0.7765 | −1.3695 | 0.0405 | −0.0115 | 9.4455 | −0.6775 | −0.7895 | −0.2485 |
| MTHFD1 | 0.2765 | 0.1965 | −0.0515 | 1.2135 | −0.4155 | 0.4495 | 0.1725 | −0.8855 | −0.1645 |
| MTMR2 | −0.0915 | −0.4005 | −0.4655 | 0.3275 | −0.4765 | 1.0565 | −1.4825 | −0.6985 | −0.9195 |
| MUC1 | 0.154 | 0.048 | −0.426 | −0.333 | 0.212 | −1.109 | 0.584 | 0.726 | 0.56 |
| MUC20 | −0.431 | −0.821 | 0.338 | −0.154 | 0.05 | 1.302 | −0.385 | −0.355 | −0.524 |
| MUM1 | 1.371 | 0.186 | 0.199 | 0.758 | 1.066 | 0.001 | 0.09 | 1.483 | 0.377 |
| MXI1 | 1.213 | 3.962 | 2.698 | −0.235 | 2.697 | 1.608 | 1.138 | 1.01 | 1.188 |
| MYB | −0.172 | 0.137 | 0.192 | −0.029 | 0.987 | 1.448 | 0.967 | 0.061 | −0.015 |
| MYBL1 | −2.407 | −2.511 | −2.075 | −0.273 | −1.699 | 0 | −2.233 | 0.023 | −0.933 |
| MYBL2 | −0.898 | −0.542 | −0.525 | 1.162 | 0.01 | −0.068 | 0.588 | 1.094 | −0.362 |
| MYO10 | 0.5015 | −0.0945 | 1.2665 | −1.2695 | 1.1115 | −0.2645 | 1.5875 | 0.7255 | 2.0605 |
| MYO6 | −2.0915 | 1.3095 | −1.7985 | 1.2775 | 1.1425 | −3.1275 | 0.7605 | −1.8465 | −1.7115 |
| MYST3 | −0.1235 | −0.2155 | 0.5295 | −0.1985 | −0.0025 | −0.0825 | 0.8155 | 0.0845 | −0.0945 |
| NALP2 | 0.3855 | 0.7935 | −0.0465 | 0.5715 | 0.8085 | −0.6585 | −0.1785 | −0.3855 | 0.3065 |
| NALP6 | 0.0975 | −0.3615 | 0.7705 | 0.3585 | −0.2495 | −0.7375 | −0.7355 | −0.2015 | −0.2435 |
| NANS | 1.471 | −0.088 | 2.793 | 1.183 | 0.374 | 1.394 | 1.902 | −0.311 | 1.292 |
| NARF | 1.345 | −0.295 | −1.454 | −0.72 | 1.128 | −2.382 | 0.908 | −0.714 | 0.228 |
| NAT2 | 0.8085 | 0.0015 | 0.2025 | 0.2335 | 0.2855 | 0.3305 | −0.8955 | 1.3115 | 0.4935 |
| NAV2 | 0.157 | 0.252 | 1.066 | 0.847 | 0.014 | 0.421 | 0.031 | 0.644 | −0.841 |
| NDE1 | −0.2205 | −0.4525 | −0.9975 | −0.1365 | −0.1375 | −1.0555 | −0.6355 | −0.6195 | −0.1345 |
| NDP52 | 1.464 | 0.99 | 0.23 | 0.54 | 0.37 | 0.571 | 1.12 | 1.073 | 0.868 |
| NDRG2 | 0.3 | −0.046 | 0.613 | 0.487 | −0.24 | 1.014 | 0.907 | 0.468 | 0.419 |
| N0UFA7 | 0.0475 | 0.1925 | 0.4155 | 0.4605 | 0.1395 | 1.5185 | 0.5815 | 1.3275 | 0.5095 |
| NDUFB1 | −0.062 | 0.26 | −0.322 | 0.502 | 0.042 | 0.314 | 0.326 | 0.158 | 0.652 |
| NDUFB10 | 0 | −0.087 | −0.125 | 0.453 | 0.213 | 1.225 | 0.73 | −0.22 | −0.024 |
| NDUFB2 | 0.1955 | −0.6605 | 0.2995 | 0.1175 | 0.3145 | 0.7545 | 1.3255 | −0.0475 | −0.3865 |
| NDUFB5 | −1.1585 | −0.0135 | −0.3735 | −0.0625 | 0.5305 | 2.0515 | 1.8035 | 3.0255 | −0.0395 |
| NDUFB6 | 0.0805 | −0.4825 | 2.8715 | 0.5835 | −0.2755 | 1.8315 | 2.0155 | 0.1995 | −0.4375 |
| NDUFC2 | 0.4285 | −0.1075 | 0.9016 | 0.2375 | 1.3725 | 2.2435 | 0.5805 | −0.4285 | 0.1225 |
| NEBL | −0.621 | −0.319 | −0.523 | −0.251 | −0.213 | −0.516 | −0.786 | −0.318 | −0.119 |
| NEK2 | 0.791 | −1.419 | 3.609 | 3.449 | 1.82 | 2.069 | 4.854 | 3.187 | 2.28 |
| NEK6 | −0.3115 | 0.7885 | 1.4325 | −0.3855 | 0.4925 | 1.0065 | 1.7855 | 0.3925 | 0.7405 |
| NELL2 | −2.1225 | −1.0705 | −0.2645 | 0.2815 | −0.1605 | 0.5045 | −0.8155 | 0.0155 | −0.7355 |
| NEURL | −0.113 | −0.333 | −0.201 | 0.775 | 1.44 | −0.399 | −3.403 | 0.34 | −1.114 |
| NFE2L3 | 0.3785 | −0.4775 | −1.2785 | −1.7765 | −1.1285 | −2.8275 | −0.7925 | −2.2515 | −1.9605 |
| NFIA | 1.855 | 1.087 | 0.467 | 1.484 | 1.221 | 0.51 | 0.305 | 0.278 | −0.029 |
| NFIB | 1.8505 | −0.0825 | −0.5575 | 0.1205 | −0.0865 | 1.0065 | 1.0905 | −0.3275 | 0.5215 |
| NINJ1 | −0.0635 | 0.2135 | 0.1605 | −0.0225 | 0.1345 | −0.4495 | −0.2095 | 0.2355 | −0.3445 |
| NIPSNAP [symbol] | 0.1085 | −0.2545 | 1.0105 | 0.0525 | 0.1235 | 0.4855 | −0.3775 | 0.1195 | 0.1065 |
| NKX8-2 | −0.2885 | −0.1755 | 0.8555 | 0.2225 | 0.1975 | 0.7615 | 0.1845 | 0.5295 | 0.1345 |
| NME1 | 0.759 | 1.01 | 0.463 | 0.808 | 0.226 | 0.62 | 0.368 | 0.735 | 0.174 |
| NME2 | 3.3385 | 0.6695 | 0.5475 | 1.6355 | 1.4935 | −0.6365 | 0.3975 | 1.7755 | 0.3895 |
| NME3 | −2.4205 | −1.0675 | −1.0335 | −0.1225 | 0.0985 | −1.0195 | 1.2605 | −1.3085 | −0.4595 |
| NMES1 | −0.441 | −0.236 | −0.111 | 0.225 | −0.208 | 1.072 | 0.341 | 0.097 | 0.522 |
| NMI | −0.2015 | 0.5325 | 0.1515 | −0.0075 | 0.4655 | 0.5715 | 0.5045 | 0.2205 | 0.6735 |
| NOC4 | 0.0445 | −0.2905 | 2.0495 | −0.5315 | −0.3545 | −0.6385 | −0.5555 | −0.3916 | −0.1485 |
| N0LC1 | −1.0215 | −0.6835 | −0.5795 | −0.9955 | −0.1235 | −0.0735 | −0.0225 | −0.3455 | −0.3235 |
| NotFound | −0.3565 | 0.0055 | −0.0545 | 0.0925 | 0.1305 | 0.4055 | 0.6065 | 0.5085 | 0.2275 |
| NotFound | −0.61 | −0.191 | 0.616 | −0.71 | −0.464 | −0.569 | 0.127 | −0.413 | −0.452 |
| NotFound | 0.559 | −0.274 | −0.228 | 0.239 | 1.477 | −0.224 | 1.564 | 0.829 | 0.12 |
| NotFound | −0.06 | −0.887 | 0.81 | 0.048 | −0.041 | −0.647 | −0.238 | −0.398 | −0.745 |
| NotFound | −1.3915 | −1.1726 | 1.2315 | 0.4855 | 0.4665 | −1.0025 | −0.5295 | −0.7775 | 0.8085 |
| NotFound | −0.343 | 0.129 | −0.324 | −0.55 | 0.869 | −0.222 | −0.763 | −0.803 | 0.227 |
| NotFound | 0.2025 | 1.7775 | 1.3555 | −1.2695 | −0.6255 | 1.6515 | 2.7685 | −0.0215 | 0.5835 |
| NotFound | −1.414 | −1.23 | −0.54 | 0.887 | −0.448 | 0.028 | 0.078 | 0.355 | −0.779 |
| NotFound | −0.7595 | −0.6975 | −0.9565 | −0.7345 | −0.4735 | −1.1345 | −0.8305 | −0.4795 | −0.6965 |
| NotFound | −1.0235 | −2.1165 | 0.7975 | −2.7735 | −1.0285 | −1.8545 | −1.9745 | −2.7545 | −2.2015 |
| NotFound | 0.6835 | 0.5445 | 0.4975 | −0.0895 | −0.1695 | −0.8035 | 0.1165 | −0.1315 | 0.1875 |
| NotFound | 0.1265 | 0.2215 | 0.0135 | −0.1585 | −0.0925 | −0.1075 | −0.0165 | −0.0535 | 0.1945 |
| NotFound | 2.127 | 1.211 | 0.268 | 1.391 | 1.731 | −2.034 | 0.227 | 0.022 | 1.468 |
| NotFound | −0.037 | −0.041 | 0.14 | 0.252 | 0.023 | 0.143 | −0.236 | −0.217 | −0.273 |
| NotFound | 0.51 | 1.112 | 1.649 | 1.001 | 0.302 | −0.668 | 0.754 | 1.414 | 0.677 |
| NotFound | 1.4775 | 1.4445 | 1.2685 | 0.3165 | 0.9085 | 1.0455 | 1.0205 | 1.2875 | 1.3375 |
| NotFound | −0.063 | 0.479 | 0.203 | 0.068 | 1.065 | −1.224 | 0.08 | 1.371 | 0.666 |
| NotFound | −0.764 | −1.254 | −0.91 | 0.033 | 0.238 | −0.029 | 0.315 | −1.107 | −0.784 |
| NotFound | −0.5115 | 0.3185 | 0.6455 | 0.0485 | 0.3885 | 0.2555 | −0.4325 | 0.0725 | 0.6095 |
| NotFound | 3.0855 | 2.7595 | 1.2935 | 0.6425 | 2.9265 | 0.6745 | −0.0245 | 1.3295 | −1.5235 |
| NotFound | −0.026 | −0.167 | 0.011 | 0.235 | 0.061 | −0.378 | −0.164 | 0.48 | 0.22 |
| NotFound | 0.0755 | −1.0765 | −0.2645 | −0.5275 | −0.9325 | 0.4415 | −0.9785 | 0.0895 | −0.1245 |
| NotFound | 1.201 | 0.122 | 3.155 | 1.945 | 1.293 | 1.885 | 2.278 | −1.295 | 3.228 |
| NotFound | 0.475 | 1.133 | 1.157 | −0.107 | −0.431 | 0.643 | 2.235 | −1.127 | 0.361 |
| NotFound | −0.7675 | −0.2495 | 0.6265 | 0.9685 | 0.2585 | 1.8595 | 0.7035 | 0.0365 | 0.4315 |
| NotFound | −0.758 | −0.148 | 0.268 | −0.06 | 0.37 | 0.135 | −0.128 | −0.424 | −0.482 |
| NotFound | 0.4085 | 1.4055 | 0.6055 | −0.4155 | −0.5655 | 0.3795 | 2.0645 | −1.5085 | 0.8595 |
| NotFound | −0.816 | 0.294 | −0.317 | −0.385 | −0.183 | −1.083 | −0.881 | −0.241 | 2.998 |
| NotFound | 0.572 | 0.059 | 0.653 | 0.768 | 0.37 | 1.045 | 0.918 | 0.515 | −0.036 |
| NotFound | −0.5085 | −0.7055 | 1.8755 | −0.6885 | −0.4495 | −0.6005 | −0.5065 | −0.6405 | −0.6245 |
| NotFound | 0.3805 | 0.8075 | 2.5855 | 0.2565 | 1.4905 | 1.4015 | 3.4355 | −1.1355 | −0.2775 |
| NotFound | 1.0455 | 1.8125 | 2.3825 | −1.8845 | −0.3855 | 1.3965 | 1.5945 | −1.4115 | 0.3515 |
| NotFound | −0.1015 | 0.8415 | 0.8435 | −0.9185 | −0.4125 | 1.0355 | 2.9725 | −1.4405 | 0.7135 |
| NotFound | 0.7505 | 0.3485 | −0.5085 | 0.0085 | −0.0495 | 2.1045 | −0.1395 | 0.1155 | −0.2545 |
| NotFound | 0.126 | 0.782 | 0.926 | −0.199 | −0.195 | 0.576 | 1.639 | −0.501 | 0.412 |
| NotFound | −0.0895 | 0.5775 | 0.3785 | −0.1745 | −0.8475 | 0.3375 | 2.5675 | −1.4795 | 1.5305 |
| NotFound | 1.0245 | 0.5235 | 0.1705 | −0.6295 | 0.1285 | −0.1115 | 0.0115 | 0.4355 | 0.9825 |
| NotFound | 0.9525 | 1.5245 | 1.5365 | 0.2425 | 0.2175 | 1.2175 | 2.8045 | −1.5125 | 0.5365 |
| NotFound | 3.3355 | 0.3855 | 1.1075 | 0.2605 | 0.2925 | −0.2595 | −0.3145 | 0.1095 | 0.5695 |
| NotFound | 0.153 | 1.483 | −0.386 | 1.299 | 0.393 | −0.911 | −0.709 | −0.614 | −0.022 |
| NotFound | 0.439 | 0.206 | 0.2 | −0.018 | −0.217 | −0.393 | −0.406 | 0.216 | 0.111 |
| NotFound | −0.013 | −0.723 | 1.293 | −0.757 | −0.636 | −0.576 | −0.284 | −0.544 | −0.586 |
| NotFound | 0.5055 | 1.4275 | 0.9125 | −0.3045 | −0.4425 | 0.5655 | 2.1085 | −1.2245 | 0.7155 |
| NotFound | 0.1845 | 0.7395 | −0.2735 | 0.9825 | −0.2175 | 3.0055 | 1.0135 | 0.5535 | −0.0715 |
| NotFound | 0.426 | −0.004 | −0.003 | −0.47 | −0.52 | −0.113 | −0.436 | −0.063 | −0.157 |
| NotFound | −0.1995 | 0.1275 | −0.1515 | −0.4675 | 0.1835 | −0.4025 | −0.0835 | −0.0125 | 0.1745 |
| NotFound | 1.0545 | −0.3975 | 1.8135 | −0.7845 | 0.4095 | −1.4495 | −0.0765 | −1.0935 | −1.0525 |
| NotFound | −0.7985 | −1.2435 | −1.4375 | −1.3145 | −0.3046 | 0.0145 | −1.4145 | −0.7825 | −1.0135 |
| NotFound | 1.4135 | 0.8065 | 0.9325 | −0.1495 | −0.3215 | 0.2935 | 0.5035 | −0.6725 | 0.0285 |
| NotFound | −1.019 | −0.739 | −0.802 | −0.05 | −0.092 | 0.151 | 0.299 | −0.139 | 0.413 |
| NotFound | −0.228 | −0.523 | 0.981 | 0.983 | 0.555 | −0.249 | 0.695 | 0.761 | −0.447 |
| NotFound | 0.1735 | 0.0885 | 0.1385 | −0.0625 | 1.0545 | −0.2105 | 1.2945 | 0.8155 | 0.4275 |
| NotFound | 0.1005 | 0.6585 | 0.6785 | 0.8225 | 0.1555 | −0.4505 | 1.2305 | 0.2285 | 0.5955 |
| NotFound | 1.3785 | 0.8515 | 1.9925 | 0.7325 | 1.1225 | 0.5415 | 0.8145 | 2.1675 | 0.6775 |
| NotFound | 0.4025 | −0.7755 | 0.2815 | −1.2505 | 0.1035 | −0.0635 | −0.2005 | −0.7155 | −0.6755 |
| NotFound | −0.552 | 0.848 | −0.699 | 1.855 | 1.464 | −1.181 | −1.277 | −1.681 | −1.647 |
| NotFound | 3.025 | −0.267 | −0.3 | −0.53 | −0.143 | 2.399 | −0.46 | 1.548 | −0.733 |
| NotFound | −0.4575 | 0.3915 | 0.3595 | −0.1545 | 0.0715 | −0.0055 | 0.0575 | 0.0885 | 0.1775 |
| NotFound | 0.779 | 0.278 | −1.395 | 1.651 | −0.639 | −0.885 | 3.975 | −0.525 | 3.219 |
| NotFound | 0.104 | 0.44 | 0.607 | −0.13 | −0.46 | 0.304 | 0.4 | −0.387 | −0.644 |
| NotFound | −1.0715 | 1.9045 | 1.3435 | −0.7845 | 2.0855 | 2.2595 | 2.6835 | −0.1525 | 0.5635 |
| NotFound | 0.092 | −0.111 | −0.207 | −0.051 | −0.044 | 0.011 | 0.08 | −0.194 | 0.027 |
| NotFound | 1.189 | 2.346 | 2.867 | −2.059 | −0.319 | 1.665 | 2.035 | −1.71 | 0.444 |
| NotFound | 0.0125 | 0.4205 | 0.8305 | 0.5795 | 1.2135 | 1.0235 | 4.0615 | 1.6255 | 3.3165 |
| NotFound | 0.698 | 0.776 | 1.026 | 1.305 | 0.814 | −0.001 | −0.979 | 0.428 | 0.001 |
| NotFound | −0.6755 | −0.3015 | 0.1875 | −0.4835 | 0.1445 | −1.1125 | −0.6065 | 0.7025 | 1.1395 |
| NotFound | −0.0375 | −0.2085 | 0.0595 | −0.2115 | 0.2755 | −0.0645 | −0.4325 | 0.0055 | 0.1785 |
| NotFound | 0.528 | 1.52 | 0.845 | −0.298 | −0.487 | 0.177 | 1.996 | −1.302 | 0.714 |
| NotFound | 0.0145 | 0.5755 | 0.0795 | 0.3405 | −0.0945 | 0.9305 | 0.4775 | −0.3875 | −0.0195 |
| NotFound | 1.014 | 1.37 | −0.196 | 0.491 | 0.571 | −0.59 | 1.547 | −0.19 | 1.887 |
| NotFound | 0.715 | 0.973 | 0.874 | 1.137 | 0.624 | 1.326 | 0.230 | 0.422 | 0.353 |
| NPDC1 | −0.2655 | 0.1735 | −0.7415 | −0.5615 | −0.2435 | −0.8585 | −0.5785 | 0.1025 | 0.0815 |
| NPEPPS | 0.755 | 0.887 | 0.807 | 0.795 | 0.073 | 0.491 | 1.358 | 0.652 | −0.229 |
| NPY | −0.22 | −0.094 | −0.014 | 0.057 | −0.166 | 0.084 | −0.366 | −0.205 | 0.234 |
| NQO3A2 | 2.081 | 0.794 | 0.659 | −0.254 | 1.416 | 0.307 | 0.786 | 1.062 | 0.387 |
| NRAS | 0.475 | −0.018 | −1.548 | 2.152 | 2.051 | 0.018 | −0.371 | −1.844 | −2.479 |
| NRIP1 | 1.0555 | 0.1135 | −0.0875 | 0.5125 | −0.3285 | 0.9975 | 0.5455 | 0.7855 | 0.1175 |
| NTN4 | −0.2875 | 0.3555 | 0.8915 | 0.3945 | 0.1355 | 0.6855 | −0.4655 | −0.0015 | −0.2475 |
| NUCB2 | 0.4245 | 0.3105 | 1.2405 | 1.9195 | 1.2685 | 1.8395 | 1.5135 | 1.3705 | 0.7705 |
| NUDT5 | −0.128 | 0.066 | 0.5 | 0.17 | 0.341 | 0.978 | 0.543 | 1.073 | −0.153 |
| NUDT6 | 0.348 | −0.542 | −0.357 | 0.075 | −0.069 | −0.377 | −0.85 | −0.586 | 0.104 |
| OCIAD2 | −0.8385 | −0.9285 | −0.2215 | 0.1235 | −0.6005 | 0.0615 | −0.8435 | −0.2025 | −0.8395 |
| OFD1 | 1.3835 | 1.7045 | 1.4495 | −0.3535 | 1.2745 | 0.2605 | −0.1385 | −0.5575 | −0.2325 |
| OGFRL1 | 0.29 | 0.278 | 0.344 | 0.075 | −0.375 | −0.424 | −0.217 | 0.285 | −0.152 |
| OPLAH | −1.4565 | −1.2505 | −0.5115 | −1.0195 | 0.3605 | −1.1995 | −2.0685 | −1.1985 | −0.3205 |
| ORMDL3 | 0.765 | −0.327 | 2.301 | −1.19 | 0.09 | −1.658 | 2.153 | 0.517 | 0.179 |
| OSBPL3 | −1.071 | −0.154 | −0.002 | −0.127 | −0.443 | 0.031 | −0.784 | −0.302 | 0.019 |
| OSR2 | −0.2025 | 0.1325 | 0.7245 | −0.2145 | −0.0035 | −0.0275 | −0.5055 | −0.2685 | −0.4345 |
| OSTF1 | −0.489 | 1.545 | −0.833 | 0.033 | 3.085 | −0.125 | 2.39 | −1.733 | −0.327 |
| P2RX1 | −0.7345 | 0.9585 | −0.0645 | −0.9675 | −0.9325 | −1.8535 | −1.7725 | −0.2195 | 0.5105 |
| P2RY2 | −1.016 | −0.336 | 0.358 | −0.526 | −0.442 | −0.455 | −0.491 | −0.183 | −0.186 |
| PACAP | 1.568 | 1.252 | 0.654 | −0.194 | −0.1 | 1.189 | −0.656 | −0.196 | 0.416 |
| PADI2 | −1.1075 | −1.5495 | −0.1095 | −0.2435 | −0.4105 | 1.9345 | −0.1435 | −0.8615 | −0.9975 |
| PAPD5 | 0.202 | 0.807 | 0.149 | 0.234 | 0.418 | 1.703 | 1.454 | 0.821 | 1.721 |
| PAPSS2 | 0.059 | 0.407 | 0.269 | 0.621 | 0.416 | 0.607 | 0.587 | −0.6 | 0.056 |
| PARD8A | −1.196 | −0.418 | 1.596 | −0.374 | 0.271 | 0.086 | 1.481 | −0.673 | −0.304 |
| PARP1 | 0.435 | 0.739 | 0.935 | 0.516 | 0.369 | 0.092 | 0.97 | 0.431 | 0.311 |
| PARP9 | 1.8525 | 1.7245 | 0.6905 | −0.1595 | 0.3325 | 0.4745 | −0.4715 | 0.3225 | 0.4745 |
| PAXIP1L | −0.4835 | −1.0455 | −1.4995 | −0.7255 | −0.6905 | −0.0565 | −1.0965 | −0.4375 | −0.6155 |
| PBX3 | −0.9735 | −0.7915 | −0.9315 | −0.2965 | 0.4745 | −0.0505 | −1.2395 | 0.6385 | −0.2415 |
| PCDHB11 | 0.56 | −0.94 | −1.328 | −0.334 | −1.045 | 0.991 | −2.491 | −0.52 | −0.501 |
| PCDHB16 | 0.461 | −0.356 | 0.27 | −0.292 | 0.691 | 0.867 | −0.458 | −1.158 | −0.895 |
| PCDHB2 | −0.6445 | 0.1605 | 0.8255 | 0.8285 | 0.6895 | 1.5465 | 0.4095 | 0.8445 | 0.5455 |
| PCM1 | −0.0105 | −0.1785 | 0.7415 | −0.3235 | −0.1825 | −0.4675 | 0.0275 | −0.0755 | −0.0465 |
| PCNA | −0.005 | −0.534 | 0.234 | 0.296 | 0.188 | 1.168 | 0.796 | −0.276 | −0.459 |
| PDC01 | 0.2525 | 0.5425 | 0.6235 | 1.8525 | 1.9715 | −0.2775 | −0.4515 | −0.3855 | 0.1955 |
| PDC010 | 0.269 | 0.194 | −0.112 | −0.046 | 0.103 | 0.637 | 0.555 | 0.9 | 0.756 |
| PDCD4 | −0.3075 | 0.5215 | 0.3755 | 0.9785 | −0.0235 | 0.6555 | 0.1995 | 0.6585 | 0.5625 |
| PDCD5 | 0.4915 | −0.0365 | −1.2195 | −0.8685 | −1.4945 | −1.9655 | −1.8995 | −0.7935 | −0.2565 |
| PDCD8 | −0.2625 | 0.0185 | 0.3855 | 0.7655 | −0.2305 | 0.3165 | −0.3665 | 0.3055 | 0.0195 |
| PDE4DIP | −0.7345 | 0.3075 | −0.3155 | 0.5175 | −0.0845 | 0.2155 | −0.7015 | 1.2295 | −0.3145 |
| PDHA1 | −0.511 | 1.367 | 0.403 | 3.252 | 2.029 | 1.634 | 0.296 | −1.508 | 2.172 |
| PECI | −0.0485 | 0.9125 | −1.0185 | 0.1285 | 0.5905 | −0.7835 | 1.0545 | 0.0155 | 0.3895 |
| PEG10 | −1.7475 | −1.6905 | −0.4445 | −1.8325 | −1.0775 | −1.2085 | −0.9955 | −0.7825 | −0.9425 |
| PER2 | −0.016 | 0.046 | 0.164 | 0.069 | −0.219 | −0.168 | 0.205 | 0.144 | 0.352 |
| PERP | −0.5895 | −0.7685 | −0.6005 | −0.6555 | −0.0705 | −0.7345 | 0.6615 | −1.0495 | −0.0435 |
| PEX1 | −0.245 | 0.18 | 0.046 | 0.053 | −0.063 | 0.727 | 0.26 | −0.41 | 0.012 |
| PFAAP5 | −0.7655 | −0.6476 | 0.3965 | 0.0385 | 0.3675 | −0.4015 | 0.7705 | −0.6825 | −0.4955 |
| PFDN2 | −0.565 | 1.837 | 0.903 | 1.11 | 0.102 | 0.517 | 0.853 | 1.046 | 0.707 |
| PGM1 | 0.2355 | −0.1485 | 0.6035 | 0.4335 | 0.0315 | 1.0195 | 0.1375 | −0.4145 | −0.0965 |
| PGR1 | −0.6855 | −1.0075 | −1.6585 | −0.9875 | 0.1185 | −0.5615 | −0.3515 | −1.1965 | −0.8705 |
| PHAX | 0.911 | 0.904 | 0.912 | 0.906 | 0.239 | 1.97 | 0.682 | 0.586 | −0.168 |
| PHGDH | 0.5155 | 0.3015 | −1.4995 | 1.2215 | 1.0345 | 0.2875 | 1.6595 | 1.0705 | 0.4715 |
| PHYH | 0.7445 | 0.1335 | 0.9195 | 0.3235 | 0.3765 | 0.9455 | 0.7555 | 0.6275 | −0.4435 |
| PI85PA | −0.6315 | −0.1325 | 0.6255 | −0.9175 | −0.5445 | −0.9245 | −1.0485 | −0.4305 | −0.4085 |
| PIGT | 0.501 | 1.111 | 0.518 | 0.12 | 0.233 | −1.254 | 0.969 | 0.621 | −0.368 |
| PIM2 | 0.0885 | −1.0195 | −0.4015 | −0.3085 | −0.2585 | −0.7045 | −1.2595 | −1.4575 | −0.6485 |
| PISD | −0.293 | −0.518 | 1.204 | 2.841 | 1.551 | 1.72 | 2.145 | 3.36 | −0.959 |
| PITPNC1 | −0.0805 | 0.4625 | 0.8225 | 0.0255 | 0.8765 | 1.3705 | 1.1035 | 0.7335 | 0.6245 |
| PKIB | 0.344 | 0.567 | 1.088 | 0.618 | 0.207 | 0.58 | 0.255 | 0.847 | 0.26 |
| PKMYT1 | −1.113 | −0.326 | 0.157 | −0.473 | −0.11 | −0.737 | −1.183 | −1.045 | −0.081 |
| PLA2G4B | 1.9955 | 0.7215 | 0.2745 | −0.1185 | 0.7395 | −0.7825 | −1.3025 | −1.1285 | −0.4065 |
| PLAC8 | −0.808 | −0.311 | 0.308 | 0.283 | 0.582 | −0.24 | −0.05 | −0.339 | 0.07 |
| PLCB1 | −1.3205 | 0.1375 | 0.4835 | 0.6035 | −0.0395 | −0.1945 | −0.1255 | −0.1885 | 0.8205 |
| PLCXD1 | −0.406 | −0.275 | −0.128 | 0.213 | −0.515 | −0.955 | −1.544 | −0.053 | 0.207 |
| PLEK2 | −1.0825 | −0.5745 | −0.1295 | 0.0845 | 0.2585 | 0.7145 | −0.2055 | −0.2545 | −0.6495 |
| PLO0 | −0.1505 | 0.3985 | −0.2955 | 1.3125 | 0.9805 | 3.2135 | 2.3845 | 0.0335 | 0.9245 |
| PNAS-4 | −1.1865 | −1.3775 | −0.7285 | −0.3575 | 0.0205 | −0.8425 | 1.2085 | −0.7875 | −0.3495 |
| PNLIPRP2 | 0.5225 | −0.2925 | 1.0795 | −1.0515 | 0.6545 | 0.2505 | 0.4065 | 0.7165 | −0.3885 |
| PNMA3 | −0.2165 | −0.1525 | −0.4845 | −0.1165 | −0.1825 | −0.6325 | 0.0615 | 0.3555 | 0.0015 |
| POLB | 0.407 | 0.439 | 0.153 | 0.302 | 0.043 | 0.473 | 0.21 | −0.138 | 0.666 |
| POLR2F | 0.4385 | −0.3015 | 0.1785 | −0.7265 | −0.2855 | −0.4515 | −1.0075 | −0.5025 | −0.1875 |
| POLR2H | −0.1755 | −0.5685 | 0.8775 | 0.1315 | −0.3215 | 0.6055 | 0.2935 | −0.0725 | −0.4605 |
| POMC | 0.317 | 0.898 | −0.103 | 0.372 | 0.179 | 0.08 | 0.605 | 0.291 | −0.058 |
| POP4 | 0.043 | −0.642 | −0.929 | −0.027 | 0.039 | −1.105 | 0.107 | 0.651 | 0.67 |
| POP7 | 0.888 | 1.026 | 0.748 | −0.533 | 1.991 | −0.656 | 2.633 | 3.501 | 3.183 |
| PP1057 | −0.5395 | −1.2465 | −0.3025 | −0.2105 | 0.0125 | −1.7975 | 0.4035 | −1.2795 | −1.0745 |
| PPFIA1 | 0.251 | 0.298 | 0.588 | 0.326 | 0.478 | 0.007 | −0.651 | −0.036 | −0.435 |
| PPFIBP2 | 0.5765 | −0.2545 | 0.4155 | 0.6335 | −0.0915 | 0.2235 | 0.2185 | 0.3645 | 0.2975 |
| PPGB | −0.5855 | 1.3565 | −0.0295 | 1.6885 | 0.3385 | 0.2005 | −0.0875 | 0.7575 | −0.2425 |
| PPHLN1 | −0.0755 | 0.0865 | −0.0525 | 0.5465 | −0.4935 | 0.7865 | 0.6325 | 0.4115 | 0.7865 |
| PPIF | −0.0525 | 0.0005 | 0.1105 | −0.0005 | 0.0245 | 0.4805 | −0.0075 | −0.1685 | −0.1625 |
| PPIL1 | 0.6235 | 1.5585 | 1.5085 | −0.7015 | 0.8005 | −0.2065 | 0.1735 | 0.4075 | 0.9495 |
| PPOX | 0.942 | 2.354 | 2.521 | 2.404 | 1.213 | −1.882 | 3.273 | −0.891 | −0.083 |
| PPP1R16 [symbol] | 0.054 | 0.08 | 0.142 | 0.332 | 0.128 | −0.02 | −0.121 | −0.13 | 0.014 |
| PPP1R3C | 0.259 | 1.461 | −0.081 | 0.409 | −0.329 | 1.001 | 0.472 | 0.081 | −0.246 |
| PPP2R2D | −0.6425 | −0.0535 | −1.2325 | −0.9285 | −0.4635 | 5.2515 | −0.6305 | 4.9745 | −0.3585 |
| PPP2R5A | 0.303 | 0.425 | 0.283 | −0.259 | 0.84 | 0.969 | 0.755 | 0.694 | 0.947 |
| PRAME | −0.4245 | −0.2275 | −0.0545 | 0.0735 | −0.2605 | 0.7795 | 0.5815 | 0.1475 | 0.3316 |
| PRC1 | −0.386 | 0.282 | −0.431 | 0.078 | 0.337 | −0.541 | 0.029 | 0.061 | −0.085 |
| PRIM2A | −1.3055 | −0.4865 | 0.1825 | −0.4995 | 0.1845 | −1.3705 | −1.5405 | −0.6885 | −0.8355 |
| PRKACB | 1.263 | −0.074 | −0.324 | 0.286 | 0.494 | 0.395 | 0.252 | −0.442 | 0.223 |
| PRKCB1 | −1.316 | −1.177 | −0.757 | −0.409 | 0.18 | 0.995 | 2.619 | 3.504 | 2.086 |
| PRKCI | 0.386 | −0.602 | 0.119 | −0.101 | 0.239 | 0.173 | −0.582 | 0.316 | 0.895 |
| PRKRIR | −0.6165 | −0.7315 | −0.8715 | −0.0935 | −0.1115 | −0.1095 | −0.7345 | −0.9325 | −0.9935 |
| PRKWNK1 | −2.0165 | −0.6695 | −1.6255 | −0.9605 | 0.0395 | 0.7245 | −0.2535 | −2.2715 | −1.5605 |
| PRKX | 0.264 | 0.377 | 0.631 | −0.399 | −0.328 | −0.336 | −0.105 | −0.062 | 0.114 |
| PRNP | −0.5985 | −0.3305 | 0.4895 | 0.3585 | 0.0695 | 0.8345 | 0.6175 | −0.3275 | −1.1615 |
| PROL5 | 0.9425 | 0.2965 | 0.5995 | −0.0045 | −0.0715 | 0.1285 | 0.3755 | −0.3455 | 0.1135 |
| PRPF18 | −1.1715 | 0.0715 | −1.0115 | 0.6245 | 0.1575 | −0.1575 | −0.7915 | 0.0315 | 0.0785 |
| PRPSAP1 | −1.6335 | −1.8155 | −3.2495 | 0.8095 | −1.5915 | −0.9105 | −0.4605 | −0.4075 | −1.9025 |
| PRSS12 | 0.7085 | −0.7965 | −1.7355 | 0.2765 | −0.6355 | −1.9665 | 0.0085 | −0.8855 | 0.8115 |
| PRSS16 | 0.0215 | −0.8525 | 0.7095 | 0.7115 | −0.6495 | 1.1365 | 0.8855 | −0.2665 | −0.1655 |
| PSD3 | 0.7035 | 1.1515 | 0.5525 | 0.3435 | 0.2775 | 0.7755 | 1.0825 | 0.0505 | 0.1535 |
| PSMA3 | 0.11 | 0.826 | 0.51 | −0.022 | −0.022 | −0.393 | −0.282 | 0.065 | 0.113 |
| PSMA7 | −0.169 | −0.825 | 0.583 | 0.681 | −0.28 | 0.743 | 0.859 | −0.026 | 0.027 |
| PSMC5 | 1.7535 | 1.3745 | 0.2495 | 0.2225 | 0.1665 | 0.6245 | 0.9625 | 1.1135 | 0.8795 |
| PSMC6 | −0.161 | 0.801 | 0.493 | 0.534 | −0.139 | 0.728 | −0.171 | −0.484 | −0.531 |
| PSMD12 | 0.8915 | 0.4215 | 1.0715 | 0.4845 | 0.0015 | −0.0085 | −0.0785 | 0.2855 | −0.5385 |
| PSMD12 | −1.0235 | −0.9605 | 0.0545 | 0.0075 | 0.4816 | −0.7635 | −1.9275 | −0.5565 | −0.9916 |
| PSMD3 | −0.7675 | −0.8545 | −0.8735 | −0.1515 | −0.3095 | 0.0795 | −0.3985 | −0.3465 | −0.9255 |
| PSPH | 1.693 | 0.097 | 0.432 | −0.61 | 0.608 | 0.14 | −0.733 | 0.626 | 0.342 |
| PTD012 | 3.793 | 0.048 | −0.255 | −0.164 | −0.45 | 0.244 | −0.695 | −0.152 | −0.204 |
| PTGER3 | 0.981 | 1.171 | 0.95 | −0.176 | 0.495 | 0.838 | 0.953 | −0.224 | −0.204 |
| PTHLH | −0.4775 | 0.0015 | −0.2885 | −0.3815 | 0.1395 | −0.5855 | 0.0985 | −0.9015 | 0.1845 |
| PTK6 | −0.786 | −0.341 | 0.87 | −0.13 | −0.29 | −0.561 | −0.359 | −0.13 | −0.178 |
| PTPN4 | 0.158 | 0.218 | −0.018 | −0.126 | −0.003 | −0.579 | −0.806 | −0.327 | −0.518 |
| PTPN7 | −0.814 | 0.185 | −0.959 | 0.051 | −0.031 | −0.77 | 3.823 | 1.308 | 2.855 |
| PTPRF | 0.408 | 1.329 | −0.291 | 1.398 | 1.715 | 2.107 | 2.294 | 1.071 | 1.714 |
| PTPRN2 | −0.1355 | −0.2265 | −0.5725 | 0.2675 | −0.4585 | 0.0385 | −0.0125 | 0.3965 | 0.2545 |
| PTPRT | 0.9575 | −0.1185 | 0.6185 | 0.0815 | 0.8695 | 1.4545 | 0.3625 | −0.0395 | 0.5715 |
| PTS | −0.802 | −0.347 | −0.065 | 0.04 | −0.016 | 1.38 | −0.053 | −0.758 | −0.732 |
| PTTG1 | 0.0516 | 0.2285 | 0.6305 | 1.1285 | 1.2965 | 1.1475 | 1.6315 | 1.0405 | 0.5145 |
| PURB | −0.2465 | 0.0715 | −0.8395 | 1.6305 | 0.7145 | 1.4825 | 0.9395 | 0.0765 | −0.0245 |
| PXMP2 | −0.0995 | −0.4515 | 0.1735 | −0.1635 | 0.0175 | 1.1035 | 0.2435 | 0.0445 | −0.2465 |
| PYCARD | 0.892 | 2.132 | 0.122 | 2.055 | 1.378 | 0.119 | 0.563 | 1.505 | 0.984 |
| PYCR1 | −0.627 | 0.225 | −1.102 | 2.055 | 0.24 | 0.951 | 1.032 | −0.917 | −0.401 |
| QDPR | 0.7445 | −0.0495 | 0.8015 | 1.9255 | −0.6175 | −0.0815 | −0.2595 | −0.7725 | −0.9575 |
| QPRT | 2.8 | 1.279 | 1.034 | −1.126 | −0.456 | 0.969 | 0.889 | 3.431 | 2.104 |
| QSCN9 | −1.1175 | 0.7865 | 1.0955 | 0.8765 | −0.3025 | 0.8516 | 0.1916 | −0.2485 | 0.2295 |
| RAB11FIP | 0.389 | 0.275 | −0.018 | 0.152 | 0.232 | 0.482 | −0.612 | −0.217 | −0.37 |
| RAB11FIP | −1.42 | −0.831 | −0.558 | −0.079 | 0.13 | 0.71 | −0.583 | −1.133 | −0.96 |
| RAB13 | 0.329 | −0.328 | 1.618 | 1.12 | 0.159 | 0.581 | −0.85 | 0.279 | −0.481 |
| RAB27A | 0.105 | −0.148 | 1.142 | 0.045 | 0.335 | 0.5 | 0.99 | −1.635 | 0.047 |
| RAB30 | −0.9525 | −0.2965 | 0.2345 | 0.6735 | −0.1535 | −0.0555 | 0.0085 | −0.3225 | −0.0285 |
| RAB34 | 1.171 | 0.281 | 0.595 | 0.417 | −0.533 | 0.311 | 2.286 | 0.313 | 3.516 |
| RAB35 | −0.062 | −0.167 | −0.22 | −0.036 | 0.005 | −0.416 | 0.092 | 0.117 | −0.206 |
| RAB39B | 0.4575 | −0.3055 | 0.4965 | −0.1315 | 0.3865 | 0.3585 | −0.1695 | 1.2245 | 1.9355 |
| RAB3A | −0.598 | −1.661 | 2.343 | 2.484 | −0.942 | 1.789 | −0.506 | 1.887 | 0.729 |
| RAB3IP | −0.69 | −1.089 | −0.107 | −0.707 | 0.082 | −0.339 | −0.476 | −1.063 | −0.782 |
| RABEP1 | 0.203 | −0.216 | 0.773 | 0.457 | 1.082 | 1.303 | 1.227 | 0.557 | 1.714 |
| RABGAP1 | 0.6225 | −0.0195 | 0.8445 | 0.2555 | 0.5055 | 0.7905 | 0.5475 | 0.7235 | 0.6875 |
| RACGAP1 | 0.0115 | 0.7416 | 0.6115 | −0.5565 | 1.4805 | 1.0795 | 1.6165 | 1.1645 | 0.6475 |
| RAD17 | 0.143 | −0.432 | 0.587 | 0.223 | 1.005 | 1.595 | 1.912 | 0.622 | 0.531 |
| RAD51 | −0.335 | −0.051 | 0.268 | −0.018 | 0.027 | −0.259 | −0.468 | 0.261 | 0.064 |
| RAD51AP | 0.1135 | 0.7915 | 0.4835 | 0.5105 | 1.6085 | −0.2675 | 0.0005 | 0.6965 | 0.4245 |
| RAD52 | 1.505 | 0.421 | 0.197 | 0.331 | 1.442 | 2.114 | 1.974 | 0.398 | 0.984 |
| RAD54L | −0.3525 | −0.1965 | 0.6255 | −0.1905 | 0.5855 | 0.0325 | 0.6445 | −0.1075 | 0.0065 |
| RAMP | −1.6015 | −1.7275 | −0.7385 | −2.0625 | −0.3665 | 0.0095 | −1.0025 | −1.9325 | −1.5125 |
| RANBP1 | −0.5825 | 0.0395 | 0.5995 | 0.4245 | 0.5255 | 1.3905 | 1.1235 | 0.8235 | 0.3975 |
| RARRES1 | −0.791 | 0.922 | −0.368 | −0.136 | 0.716 | 2.06 | 0.486 | 2.271 | 0.834 |
| RBBP7 | −0.8495 | −0.5785 | 0.0755 | 0.4375 | 0.0335 | 0.6695 | −0.3725 | −1.0705 | −0.6125 |
| RBBP8 | −0.5485 | −0.4855 | 0.2145 | 0.0495 | 0.4105 | 0.1625 | 0.8535 | 0.4415 | −0.5035 |
| RBM8A | 0.379 | −0.542 | 0.294 | −0.35 | 1.314 | 1.164 | 0.412 | −0.16 | −0.521 |
| RBX1 | 0.601 | −0.788 | 0.724 | 0.56 | 0.162 | −0.006 | 0.059 | 0.002 | −0.057 |
| RDHE2 | −0.014 | 0.661 | 0.405 | −0.408 | 0.411 | 0.657 | 1.092 | 0.359 | 0.435 |
| REA | −0.042 | 0.035 | −0.192 | −0.31 | −0.323 | 0.072 | 0.123 | −0.089 | 0.106 |
| REC14 | −0.6985 | −0.8005 | −0.5355 | 0.0815 | −0.4825 | −0.7285 | 0.4125 | −0.1965 | −0.9845 |
| REG1A | −0.01 | 0.899 | 0.623 | 0.323 | 0.087 | −0.239 | 0.507 | 0.18 | 0.329 |
| RELB | 0.082 | 0.664 | 3.053 | 1.112 | 1.265 | 2.075 | 2.982 | 0.976 | 0.546 |
| REPIN1 | 0.194 | 0.337 | 1.305 | 1.51 | 0.176 | 0.955 | 1.805 | 0.984 | 1.035 |
| REPS2 | 0.0845 | −0.1395 | 0.0475 | 0.0635 | 1.1015 | 1.1525 | 1.3785 | −0.1355 | 0.6515 |
| RERG | −0.6725 | −0.5225 | −0.1595 | 0.2265 | 0.1485 | 0.1695 | −0.1945 | 0.3745 | −0.0365 |
| RFC4 | −1.2625 | −0.3535 | −0.7135 | 0.1315 | −0.0405 | −0.0805 | −0.4485 | −0.1995 | 0.4255 |
| RGS10 | 0.4615 | 0.0435 | 1.0115 | 1.0445 | 1.7605 | 1.9085 | 1.8335 | 1.0735 | 0.2745 |
| RH8DL2 | −0.567 | −0.217 | 0.455 | 0.175 | 0.598 | 0.036 | 0.555 | 1.054 | 0.654 |
| RIBC2 | −0.766 | −0.019 | −0.095 | 0.309 | −0.027 | 1.115 | −0.285 | −0.305 | −0.478 |
| RIC-8 | 1.528 | −0.283 | 0.879 | 2.772 | 2.094 | 1.169 | 1.85 | −0.093 | −0.205 |
| RIT1 | −0.829 | 0.334 | 0.05 | 0.231 | −0.466 | −0.386 | −0.511 | 0.281 | 0.062 |
| RLN2 | 0.171 | 0.87 | 0.875 | 0.894 | 0.566 | 1.185 | 0.517 | 0.743 | 0.202 |
| RNF10 | −0.3335 | 0.2175 | 0.4325 | 0.4725 | 0.2035 | 0.3135 | 0.1585 | 0.1105 | 0.0095 |
| RNF135 | −0.8165 | 0.0365 | −1.3045 | −0.2605 | −0.5945 | −0.6645 | −0.4165 | −0.8275 | −0.8475 |
| RNF7 | −0.66 | −0.425 | 0.548 | 0.531 | 0.591 | 2.335 | 0.884 | 0.742 | 0.099 |
| ROPN1 | −0.6595 | −0.8245 | −1.5775 | 1.1955 | 1.3535 | −1.5765 | 0.0175 | −0.6485 | −1.5605 |
| RPA3 | −0.5685 | −1.2105 | −0.2375 | 0.1875 | 0.3755 | −0.5745 | −0.5365 | −0.2265 | −0.0965 |
| RPESP | −0.593 | −0.399 | −0.254 | −0.9 | −0.138 | −0.248 | −0.514 | −0.249 | 1.163 |
| RPL11 | 0.201 | −0.405 | 0.215 | −0.26 | 0.18 | −0.543 | −0.269 | 0.142 | −0.188 |
| RPL13 | −0.424 | 0.301 | −0.2 | 0.503 | 0.47 | 0.402 | 0.928 | 0.884 | 0.994 |
| RPL13A | 0.2135 | −0.4535 | −0.8595 | −0.2425 | 0.4215 | −0.5845 | −0.4855 | −0.2705 | −0.2545 |
| RPL15 | −0.29 | −0.318 | 0.647 | 0.449 | 0.02 | −0.02 | −0.194 | 0.069 | −0.186 |
| RPL18A | −1.243 | −1.001 | −0.981 | −0.341 | −0.22 | −0.301 | 0.425 | −1.129 | −0.49 |
| RPL19 | −0.804 | −0.976 | 1.031 | 0.398 | 0.168 | 0.697 | 0.533 | 0.111 | −0.445 |
| RPL21 | −0.6165 | −0.6425 | 0.9135 | 0.4015 | 0.2745 | 0.2925 | 0.5235 | 0.0555 | −0.2185 |
| RPL23A | −0.843 | −1.149 | −0.626 | 0.979 | 0.062 | −0.1 | 0.042 | 0.354 | −0.08 |
| RPL23A | −0.2675 | −0.7835 | 0.0205 | 0.0145 | 0.4495 | 0.0325 | 0.2735 | −0.0095 | −0.1065 |
| RPL26L1 | 0.0185 | 0.0025 | −0.7845 | −0.0025 | 0.5215 | −0.9465 | 0.1345 | 0.1395 | −0.1245 |
| RPL29 | −0.0535 | −0.5725 | 0.0365 | 0.4725 | 0.0495 | 0.1975 | 0.3765 | −0.1675 | 0.1185 |
| RPL35 | −0.8385 | −0.8585 | 0.1175 | −0.2565 | 0.2045 | 0.0155 | −0.1225 | −0.4515 | −0.3305 |
| RPL35AL | −0.7255 | 0.2085 | −0.8095 | −0.2325 | 0.0365 | −1.9245 | −2.1375 | 5.5965 | −0.0585 |
| RPS16 | −0.3095 | −0.3685 | −0.3205 | 0.3455 | 0.0835 | −0.0935 | 0.8145 | −0.1705 | −0.1545 |
| RPS4Y1 | −0.3915 | −1.1195 | −0.0845 | −0.2235 | −0.3325 | 0.0565 | 0.0345 | −0.7845 | −0.4585 |
| RRAGA | −1.109 | 0.231 | 0.822 | −0.057 | 1.309 | 0.913 | 1.388 | 1.536 | 1.648 |
| RRAGD | −0.671 | 0.244 | −0.106 | −0.036 | 0.433 | 0.619 | 0.676 | −0.068 | 0.509 |
| RRM1 | −0.405 | −0.314 | −0.328 | −0.287 | −0.178 | −0.137 | −0.003 | −0.354 | 0.17 |
| RRM2 | 1.206 | −0.382 | −0.653 | 0.368 | 1.568 | −1.367 | 1.789 | −2.728 | 0.853 |
| RSAFD1 | 0.0315 | −0.2745 | −0.2335 | −0.2225 | −0.5335 | −0.3515 | −1.0245 | −0.4545 | −0.6755 |
| RSNL2 | −1.4035 | −0.5875 | −0.2805 | 0.8545 | −0.2865 | 0.1205 | −0.5805 | −0.1285 | −0.6885 |
| RSU1 | 0.011 | −0.039 | −0.005 | −0.345 | 0.02 | −0.127 | −0.547 | −0.933 | −0.851 |
| RTN4IP1 | 0.794 | −1.959 | −1.286 | −0.583 | −0.164 | −0.63 | 0.649 | 0.614 | 0.09 |
| S100A11 | 0.156 | −1.999 | −2.426 | −1.15 | −0.58 | 0.256 | 0.304 | 0.191 | −0.805 |
| S100A14 | −1.0195 | −1.4715 | −1.3165 | −1.4945 | −0.0295 | −1.1375 | −1.9775 | −2.1175 | −1.1335 |
| S100A16 | 0.1935 | −0.5195 | 0.9035 | 0.2905 | 0.1705 | 0.4465 | 0.2515 | 0.6315 | 0.6025 |
| S100A9 | −0.1 | −0.42 | −0.459 | 1.717 | 1.584 | 1.468 | 1.377 | 0.286 | 0.467 |
| SAE1 | −0.3885 | −0.0485 | −0.3605 | 0.7265 | 0.3445 | 0.9195 | −0.3565 | −0.3695 | −0.1625 |
| SALL2 | −0.032 | 0.521 | 0.835 | 0.739 | −0.087 | 1.734 | 0.352 | −0.219 | 0.363 |
| SARA1 | 0.3285 | 0.0995 | 0.1145 | −0.0545 | 0.0915 | 0.1835 | 0.1575 | 0.6445 | 0.8545 |
| SARA2 | −0.467 | −0.334 | 1.05 | −0.037 | −0.021 | −0.213 | −0.485 | −0.109 | 0.158 |
| SAS | −0.64 | 1.013 | 0.565 | 0.893 | 0.56 | −1.924 | 0.799 | 1.456 | −0.595 |
| SCIN | −1.6735 | −0.9705 | −0.1885 | −0.1225 | 0.1665 | −0.9935 | −0.7936 | −0.8285 | −1.0395 |
| SCML1 | 0.517 | 1.34 | 1.183 | −0.696 | 0.903 | −0.159 | 0.288 | 0.555 | 0.223 |
| SCPEP1 | −1.4255 | 1.1335 | −0.4315 | −0.0285 | −0.7615 | −0.5275 | −0.2965 | −0.5345 | −0.5225 |
| SCRIB | −0.231 | 0.884 | −0.557 | 0.437 | 0 | −1.568 | −0.288 | −0.125 | 1.353 |
| SDC1 | −0.477 | −0.572 | −0.121 | 0.288 | 0.233 | 0.157 | 0.031 | −0.897 | −0.37 |
| SDC3 | 0.3985 | 0.2235 | −0.2455 | −0.0925 | 0.2835 | 1.1085 | 0.5945 | −0.3285 | 0.1785 |
| SDCCAG0 | −0.2665 | 0.3805 | −0.1815 | 0.1575 | 0.0445 | −0.5675 | −1.0355 | 0.1535 | −0.1275 |
| SEC61G | 0.258 | 0.019 | 0.273 | 0.233 | −0.504 | 0.061 | 1.123 | 0.383 | −0.492 |
| SECP43 | 2.2065 | 2.1925 | −0.7595 | 0.8755 | 0.5465 | −0.5535 | −2.0025 | 1.5585 | 0.0495 |
| SEDLP | −0.217 | 0.704 | −1.091 | −0.263 | 0.815 | 1.16 | 0.454 | 0.187 | −0.928 |
| SEMA3B | 1.6625 | 0.2295 | 0.5425 | −0.2965 | 0.3805 | 0.6785 | 0.7515 | −0.0265 | 0.1095 |
| SEMA3C | 0.0685 | −0.0475 | 0.2385 | 0.4125 | 0.0055 | 0.2085 | 0.1145 | 0.7105 | −0.8885 |
| SEPW1 | 1.368 | 2.29 | 4.004 | 0.189 | 0.134 | 1.954 | 2.225 | 2.686 | 0.099 |
| SERP1 | −1.0785 | 0.1265 | −1.7876 | 0.0405 | −0.3265 | −0.4145 | −1.8535 | −0.4945 | −0.1645 |
| SERPINA [symbol] | −1.611 | −1.129 | −2.228 | −2.192 | 0.432 | −2.128 | −3.273 | −2.026 | −1.371 |
| SERPINB [symbol] | −0.186 | 0.055 | −0.333 | 0.893 | −0.227 | −0.137 | −0.337 | −0.634 | −0.108 |
| SFRP1 | −0.798 | −0.698 | −0.463 | 0.214 | 0.168 | −0.774 | −1.213 | 0.54 | 0.233 |
| SFRS6 | −1.5325 | 0.7515 | −1.1165 | 0.3505 | 0.1865 | −0.0505 | 0.4735 | −0.5765 | 1.5895 |
| SGCD | 1.3835 | −0.1035 | −0.2575 | 1.4095 | 2.1755 | 1.1205 | 0.8685 | −1.1285 | −1.1435 |
| SGCE | 0.028 | −0.028 | −0.373 | 0.204 | −0.159 | 0.296 | 0.292 | −0.231 | −0.181 |
| SH3BGRL | 0.765 | 0.585 | −0.004 | 0.866 | 0.234 | −2.235 | 0.304 | −0.095 | −0.304 |
| SHFM1 | 0.0475 | 0.0345 | −0.5145 | −0.3205 | −0.0045 | −0.4335 | −0.0025 | −0.0825 | −0.2115 |
| SIAT7B | −0.977 | −0.294 | 0.679 | −0.8 | −0.207 | −1.458 | −1.042 | −1.08 | −0.428 |
| SIGLEC12 | 0.02 | 0.1 | −0.611 | 0.12 | 0.138 | −0.182 | 0.615 | 0.237 | 0.655 |
| SIT | 0.6845 | −0.6325 | 0.6835 | 0.2655 | 0.3575 | 1.3165 | 1.1075 | −0.1965 | 0.1115 |
| SITPEC | 0.2295 | 0.3335 | −0.0225 | 0.0005 | −0.0505 | −0.7785 | −0.2905 | −0.1355 | 0.1035 |
| SKP1A | −0.5885 | −0.2925 | −1.4975 | −1.1755 | −0.4615 | −1.4545 | −1.7125 | −1.4325 | −1.2215 |
| SLC12A9 | 4.008 | 1.267 | 2.146 | 0.748 | 0.413 | 1.122 | 1.247 | 1.498 | 0.133 |
| SLC13A3 | 1.4835 | 2.3235 | 2.2925 | 1.0075 | 1.1195 | 1.3805 | 0.3855 | 1.4775 | −0.6645 |
| SLC16A6 | −1.736 | −0.108 | 2.081 | −0.752 | 4.089 | 0.912 | 2.507 | −0.896 | −0.349 |
| SLC19A2 | 2.4385 | 2.8385 | 0.0355 | −0.1735 | −0.4065 | 3.4435 | 0.5725 | 3.3865 | 2.9205 |
| SLC1A1 | −0.8265 | −0.6395 | 2.3535 | 0.1775 | 0.4085 | 0.5895 | 0.2315 | −2.1935 | 1.1085 |
| SLC1A2 | 0.3115 | −0.4515 | −0.2925 | 0.0225 | −0.8065 | −1.5425 | −0.2226 | −0.2785 | −1.2195 |
| SLC24A3 | −0.28 | 0.627 | 0.392 | 1.083 | −2.452 | 0.405 | 0.994 | 0.701 | 0.808 |
| SLC25A29 | −0.709 | −0.68 | 0.533 | 0.928 | 0.097 | 1.467 | 0.37 | 0.206 | −0.124 |
| SLC25A4 | 3.252 | 0.306 | 3.08 | −0.364 | 2.532 | 1.995 | 0.684 | 3.112 | 0.322 |
| SLC25A5 | 0.1885 | −0.1735 | 1.1495 | 0.5355 | 0.5625 | 1.1605 | 0.8875 | 1.3876 | −0.0935 |
| SLC27A2 | −0.316 | 0.359 | −0.132 | −0.277 | −0.171 | −0.139 | −0.438 | 1.919 | 1.86 |
| SLC27A5 | 0.12 | −0.656 | 2.216 | −0.485 | 0.949 | 0.863 | 1.446 | 1.063 | 0.127 |
| SLC30A8 | 2.2715 | 2.4895 | 3.8505 | 1.8975 | 3.6155 | 2.1755 | 4.4555 | 0.8665 | −0.2735 |
| SLC39A4 | −0.685 | −1.37 | −0.437 | 0.313 | 2.59 | −0.507 | −2.546 | −1.693 | 0.393 |
| SLC39A6 | −0.4255 | −0.1555 | 0.0995 | −0.4075 | −0.0285 | −0.2045 | 0.3855 | 0.5745 | 0.3085 |
| SLC40A1 | −0.0426 | 0.1055 | −0.0145 | −0.5435 | −0.6155 | −1.8635 | −0.8255 | −1.5005 | −1.1305 |
| SLC41A3 | 1.298 | −0.158 | 1.51 | 0.052 | 1.018 | −0.268 | −0.387 | −0.06 | 0.593 |
| SLC5A6 | 1.6935 | 1.8665 | 2.2285 | 1.1445 | −0.0135 | 1.8555 | 0.3255 | 1.5585 | 0.8345 |
| SLC6A4 | 1.6815 | −2.8105 | 2.5315 | −2.0795 | 1.7635 | 5.8605 | 2.2575 | 0.2955 | −2.4895 |
| SLC9A3R1 | 0.8745 | 0.8965 | −0.4875 | −0.0015 | 0.2375 | 3.2825 | −0.0415 | 2.1095 | −0.2975 |
| SLITRK6 | −1.159 | 0.24 | 0.2 | 0.249 | 0.273 | 0.401 | 0.106 | −0.026 | −0.159 |
| SMA3 | −0.423 | −0.928 | −1.296 | 0.081 | −0.072 | −0.294 | −0.964 | −0.564 | −0.83 |
| SMAD2 | −0.432 | −0.546 | −0.597 | 0.119 | −0.455 | −0.13 | 0.766 | −0.597 | −0.478 |
| SMO | −0.127 | −0.226 | −0.712 | 0.757 | −0.488 | −0.759 | −0.758 | −0.147 | 0.058 |
| SMU1 | −0.888 | 0.285 | 0.676 | 0.08 | −0.15 | 1.107 | −0.407 | −0.004 | 0.401 |
| SNPH | −0.806 | −0.515 | 0.498 | 0.317 | 0.146 | 0.796 | 0.602 | −0.484 | 0.06 |
| SNRPB2 | −0.3865 | 0.8145 | 0.3025 | 0.5285 | 0.3915 | 0.0955 | 0.7705 | −0.2195 | −0.3385 |
| SNRPD1 | 0.228 | −0.504 | −0.135 | 0.155 | 0.073 | 0.45 | 0.605 | 0.297 | −0.044 |
| SNRPD3 | −0.3625 | −0.6315 | −0.3045 | −0.2075 | 0.9955 | 0.5385 | 0.6145 | 0.6985 | −0.2775 |
| SNRPG | −0.8985 | −1.5045 | 0.9335 | −0.1345 | −0.6785 | 0.2875 | −1.5835 | −1.3265 | −1.8545 |
| SNX7 | 1.1055 | 0.2105 | −0.2805 | 0.3425 | 1.1135 | 0.0575 | 0.3635 | −0.1135 | 0.0045 |
| SO02 | 1.2145 | −0.0885 | −0.3955 | −0.6795 | 0.3035 | −1.6215 | −1.6845 | −1.5125 | −0.6775 |
| SORCS1 | −2.231 | −1.76 | −0.473 | −0.104 | −0.322 | −1.152 | −0.059 | −1.103 | −0.553 |
| SOX4 | 1.3705 | −2.1105 | 2.1935 | 0.0405 | 1.5915 | 1.1645 | 2.2935 | 1.9275 | 2.3035 |
| SOX9 | 0.326 | 0.575 | 1.282 | 0.185 | 0.982 | 1.2 | 0.426 | 0.26 | 0.799 |
| SPAG18 | −0.0665 | −0.5245 | 0.0785 | −0.1485 | 0.4235 | 0.7845 | −0.0525 | 0.0155 | 0.0745 |
| SPAG5 | 1.3115 | 0.5205 | 0.8785 | −0.8245 | −0.1065 | 0.1285 | 0.8665 | 2.9505 | 2.6735 |
| SPC18 | −0.097 | −0.161 | −0.331 | −0.019 | −0.455 | −1.519 | 0.441 | −0.238 | 1.135 |
| SPFH2 | −0.527 | 0.544 | 0.247 | 0.949 | 0.055 | 0.024 | 0.355 | 0.129 | 0.205 |
| SPG3A | 0.285 | 2.975 | 1.865 | −0.595 | 0.365 | 0.769 | 1.325 | 0.175 | 0.767 |
| SPPL3 | −0.541 | −0.324 | 0.607 | 0.726 | 0.62 | 0.833 | 0.346 | −0.125 | −0.526 |
| SQLE | −0.3 | 0.358 | 1.391 | 1.222 | −0.42 | 0.21 | 0.373 | 2.184 | 1.14 |
| SQRDL | 0.9416 | 0.0485 | 0.3545 | −0.1625 | 0.7065 | −0.2045 | 2.0355 | 0.6355 | 0.5055 |
| SREBF1 | −0.4255 | 0.3565 | −0.7325 | 0.0425 | −0.2945 | 0.3985 | 0.5575 | 0.3295 | 0.8145 |
| SRI | −0.3355 | 2.2285 | −0.7595 | 0.9525 | 0.5925 | −0.5255 | 0.1705 | 0.0545 | −0.0145 |
| SRPK1 | −0.6025 | 0.0925 | −0.2425 | −0.2545 | −0.2905 | 0.1225 | −0.3285 | −0.3085 | 0.0725 |
| SSFA2 | 0.2515 | 0.1305 | −0.3955 | 0.1995 | 0.1385 | 0.8715 | 0.5055 | −0.4145 | 0.7425 |
| SSR4 | −0.0405 | −0.3905 | 0.0065 | 0.1815 | 0.1775 | −0.9685 | −0.1735 | −0.9655 | 0.3115 |
| STARD3NI | −1.3155 | −0.3676 | 1.4405 | −0.5055 | 0.3115 | −0.8485 | 1.9405 | −1.3275 | 0.0325 |
| STARD7 | 2.2875 | 1.8216 | 3.9455 | 1.4255 | 2.4425 | −0.7635 | −2.9065 | 4.1225 | −1.2725 |
| STAT1 | 0.6235 | 0.3835 | −0.4285 | −1.1175 | −0.3535 | −0.4985 | 0.5045 | −0.2665 | 0.3755 |
| STC2 | −0.0195 | 0.8505 | 0.6575 | 0.0505 | 0.8515 | 0.4975 | −0.1645 | −0.0725 | 0.2645 |
| STK24 | 0.7765 | −0.6595 | 0.2985 | 0.5065 | 1.0705 | 1.1585 | 0.1675 | 0.5315 | 1.2225 |
| STK6 | −0.076 | 0.275 | −0.477 | 0.02 | −0.031 | −0.985 | 1.162 | −0.389 | 2.198 |
| STMN1 | 0.2225 | 1.0675 | 0.1125 | −0.5085 | −0.5075 | 0.1125 | −0.3825 | −0.0905 | 0.1705 |
| STX1A | −0.4105 | −1.1505 | 0.1715 | 0.5655 | −0.8175 | 1.0045 | −0.5165 | −0.5305 | −0.5845 |
| STX3A | −0.5215 | −0.7855 | −0.1705 | −0.8005 | −0.4255 | −0.3775 | 0.5855 | −0.6755 | 0.1685 |
| STYX | −0.8415 | 0.7545 | 0.7335 | 0.0025 | 0.0045 | 0.9845 | 0.0515 | 0.0535 | 0.8625 |
| SUGT1 | −0.15 | −0.293 | 0.296 | 0.151 | −0.181 | 0.418 | 0.642 | 0.008 | −0.189 |
| SUMF1 | −0.1215 | −0.3335 | 0.6685 | 0.5325 | 0.2415 | 1.3695 | 0.4655 | −0.2105 | −0.1365 |
| SUMO2 | −0.5965 | 0.4745 | 0.1665 | 0.6765 | −0.2915 | 0.9035 | −0.4555 | 0.6425 | −0.4475 |
| SUV39H2 | 0.0785 | 1.6125 | 1.5555 | 0.3655 | 0.9945 | 1.1505 | 0.7805 | −0.0035 | −0.8975 |
| SYAP1 | −0.9225 | −0.3265 | −0.8425 | −0.6155 | 0.0405 | 0.1775 | −2.1725 | −0.7145 | −1.0355 |
| SYCP2 | 1.299 | 1.253 | 0.121 | 1.095 | −0.378 | −0.808 | −1.317 | 0.192 | 0.593 |
| SYNGR1 | 0.0415 | 0.2415 | 0.1415 | 0.4515 | 0.2385 | 0.2175 | 0.7975 | 0.3895 | 0.1655 |
| SYTL2 | −0.2115 | 0.0455 | −0.0895 | −0.1118 | 0.1685 | −0.0865 | 0.0275 | 0.1685 | −0.0765 |
| TAF18 | −0.0205 | 0.7075 | −0.4355 | −0.4945 | −0.0175 | −0.8135 | −0.2245 | −0.4665 | −0.5395 |
| TAF8 | −0.179 | −0.302 | 0.056 | −0.173 | −0.222 | 1.226 | 0.287 | −0.07 | 0.122 |
| TARBP1 | 2.279 | 0.69 | 1.021 | −0.528 | 0.392 | 0.765 | 1.165 | −0.186 | −0.115 |
| TARS | −0.1075 | 0.3045 | 0.3895 | 0.5815 | 0.5065 | 0.7415 | 1.3765 | 0.6935 | 0.3525 |
| TATDN1 | −0.2445 | 0.4355 | 0.2305 | 0.4735 | −0.0335 | 0.4885 | 1.3725 | 1.0995 | 0.0455 |
| TBC1D12 | 0.8435 | −0.4025 | 1.0275 | 0.3905 | −0.0825 | −0.5875 | −0.8145 | −0.1376 | −0.1625 |
| TBC1D14 | 1.199 | 0.043 | 1.466 | 0.64 | 0.129 | 1.281 | 1.045 | 0.625 | 0.144 |
| TBC1D3 | 1.5525 | −1.9405 | 1.8785 | 1.1535 | 2.4085 | 0.8115 | −0.2515 | −0.6435 | −0.5325 |
| TBCA | 2.088 | 0.723 | 0.866 | 1.95 | 0.75 | 0.884 | 1.907 | 0.642 | 2.407 |
| TBX1 | 0.4485 | 0.5375 | 0.2985 | 0.1555 | −0.3085 | −0.4075 | −0.0545 | −0.0735 | 0.9505 |
| TBX3 | −0.263 | −0.247 | 0.386 | 2.77 | 1.349 | 1.975 | 2.426 | 1.76 | 0.032 |
| TCAP | −0.148 | 0.162 | 1.036 | 2.303 | 0.851 | 1.491 | 1.727 | 2.121 | 0.063 |
| TCEAL1 | −0.9105 | −0.2235 | 1.1425 | 2.8565 | 0.9265 | 1.7345 | 1.3865 | 2.0535 | 0.1145 |
| TCEAL3 | 0.923 | 1.226 | 1.333 | −0.7 | −0.145 | −0.397 | −1.14 | 0.558 | 1.437 |
| TCEAL4 | −0.573 | −0.282 | −1.079 | −0.309 | 0.496 | −0.818 | −1.383 | −0.752 | −0.693 |
| TCF15 | −0.529 | −0.94 | −2.058 | −0.146 | −0.498 | −0.162 | −1.845 | −0.248 | −1.178 |
| TCF3 | 0.636 | 0.738 | −0.081 | 0.345 | 0.649 | −0.554 | 1.244 | −0.2 | 0.628 |
| TCF7L2 | −0.0225 | −0.0955 | 0.8385 | −0.4345 | 0.4815 | −0.2475 | −0.1565 | 0.4615 | −0.2075 |
| TDRD7 | 1.8695 | −3.3695 | −0.6815 | −0.3445 | 1.2135 | −3.8455 | 1.0515 | −4.2115 | 1.6925 |
| TESK2 | 0.994 | 0.041 | 1.072 | −1.077 | −0.285 | −1.485 | −1.93 | 0.206 | −0.615 |
| TFAP2B | 0.074 | −0.13 | −0.059 | −0.778 | −0.08 | −1.333 | −0.032 | −0.684 | −1.135 |
| TFCP2L1 | 2.0155 | 2.8365 | −1.0125 | 1.7385 | −0.0325 | −1.6705 | −1.6915 | 0.7975 | −0.1065 |
| TFCP2L2 | 2.4215 | 1.6515 | 1.5905 | −1.8595 | −0.9575 | 0.2995 | 0.0575 | 2.7765 | 1.5355 |
| TFF1 | −0.8755 | −0.6355 | 1.2465 | −0.2915 | 1.1905 | 0.5675 | −0.6195 | −1.2515 | −0.2205 |
| TFF3 | 1.338 | −0.464 | −0.806 | 0.131 | 0.583 | 1.217 | 0.411 | −0.826 | −1.143 |
| TFRC | −1.0185 | −0.4245 | −0.5015 | 0.0055 | −0.0715 | −0.6475 | −0.4275 | −0.0945 | −0.4195 |
| TGFBR3 | −0.036 | 0.228 | 0.461 | 0.209 | 0.054 | −0.154 | 0.487 | 0.11 | 0.072 |
| THY28 | 0.4455 | −0.3475 | 0.6525 | 0.2295 | 0.1525 | 0.0775 | 1.2535 | −0.2265 | 0.2275 |
| TIF1 | 1.058 | 0.728 | 0.53 | 0.669 | 0.052 | 1.677 | 2.789 | 0.546 | 2.5 |
| TIGA1 | −0.322 | 1.007 | 0.807 | 1.062 | 1.204 | 1.4 | 0.984 | 1.3 | 1.661 |
| TIM14 | 0.0275 | −0.6615 | 0.3475 | 0.4385 | −0.2975 | 0.6875 | 1.0555 | 0.0755 | 0.6845 |
| TIMELES [symbol] | −0.1815 | 0.1685 | −0.0505 | 0.1575 | 0.2765 | 0.6115 | 0.3535 | 0.3495 | 0.2725 |
| TIMM9 | 0.272 | −0.01 | −1.37 | 1.328 | 0.548 | 1.114 | 1.513 | 0.625 | 1.624 |
| TINP1 | 0.6185 | 0.1795 | 0.9455 | 0.5765 | 1.1665 | 1.8605 | 1.1625 | 1.4075 | 0.3245 |
| [symbol] IP1208 | 0.601 | 1.456 | −0.406 | −0.931 | 1.035 | −0.79 | 0.799 | 0.152 | 0.429 |
| TK1 | 0.003 | −0.091 | 0.633 | 0.272 | 0.439 | 0.074 | 0.067 | −0.488 | −0.196 |
| TLE2 | −0.881 | −0.67 | 0.415 | 2.018 | 0.134 | 0.601 | 2.385 | −0.236 | −0.662 |
| TLR5 | 0.585 | 1.242 | 1.104 | 0.312 | 1.106 | 1.427 | 1.176 | 0.509 | 0.938 |
| TM4SF12 | −0.203 | 0.522 | −0.079 | 0.281 | −0.25 | 1.028 | 1.09 | −0.133 | −0.061 |
| TM4SF13 | 0.085 | 0.784 | 0.014 | 1.087 | −0.054 | 0.888 | 1.361 | 1.297 | 0.149 |
| TMEM14A | −0.1355 | 0.1165 | −0.1785 | 0.5675 | 0.3545 | 0.3925 | 0.4295 | 0.1075 | 0.3655 |
| TMEM25 | 0.1385 | 0.0195 | −0.2815 | −0.0535 | −0.4345 | −1.3725 | −0.9465 | −0.2895 | −0.6765 |
| TMEM4 | −0.889 | −0.259 | −0.388 | 0.223 | −0.297 | −1.612 | −1.488 | −1.372 | −0.88 |
| TMPRSS5 | −0.647 | −0.631 | −1.993 | 0.545 | 0.541 | 2.42 | 0.94 | 0.024 | −0.17 |
| TMSB10 | −2.293 | −0.821 | −0.083 | −1.533 | −0.679 | −2.202 | −2.918 | −0.853 | −1.084 |
| TMSNB | −0.6085 | −0.7795 | −0.6815 | −0.1115 | −0.0195 | 0.3515 | −0.0705 | −0.5605 | −0.0395 |
| TNFRSF1 [symbol] | −0.361 | −0.72 | −0.835 | −0.702 | −0.278 | −0.196 | −0.628 | −0.653 | −0.404 |
| TNFRSF2 [symbol] | −1.2985 | −1.0325 | −0.1595 | 1.8865 | 0.2385 | 0.0285 | 0.0935 | 0.1165 | 1.0935 |
| TNFRSF2 [symbol] | −0.8185 | −0.7355 | 0.0325 | −0.8815 | −0.0175 | −0.8615 | 1.3905 | −0.9435 | −1.1775 |
| TNFSF4 | 0.1925 | 1.4745 | −1.2875 | 1.4245 | −0.1545 | 0.5395 | 0.0045 | −0.4165 | −0.0945 |
| TNFSF7 | −0.2275 | 0.1005 | 1.1965 | −0.3925 | −0.5045 | 0.4045 | −0.7405 | 0.2585 | 0.7185 |
| TNIK | 0.0295 | −0.0115 | −0.6555 | −0.6305 | −0.5285 | −1.1585 | −1.1065 | −0.7385 | −0.5425 |
| TNNC2 | 0.433 | −0.533 | −1.225 | −0.674 | 1.749 | 3.301 | 4.516 | 4.18 | 3.027 |
| TNNI2 | 0.722 | 0.585 | 0.915 | 0.399 | 0.182 | −0.329 | −0.661 | 0.17 | −0.104 |
| TNNI3 | 0.8025 | 0.1475 | 0.7495 | 0.6625 | 0.5335 | 0.2075 | 1.2315 | 0.5775 | 0.8655 |
| TOB1 | −0.2055 | −0.8665 | −0.2745 | 0.0875 | 0.1125 | 0.3685 | 0.3205 | 0.0305 | −0.1755 |
| TOM1L1 | −0.5645 | 0.2705 | 0.9235 | −0.7745 | 0.1795 | −0.6885 | 0.0145 | −0.3665 | −0.0385 |
| TOMM7 | 1.1425 | 0.7905 | 1.6455 | 1.1475 | 1.8905 | 1.7545 | 1.0945 | 0.8265 | 1.6305 |
| TOP1MT | −1.239 | −0.208 | −0.267 | −0.158 | 0.429 | −0.213 | −0.116 | 0.222 | 0.287 |
| TOP2A | 0.3535 | 0.2555 | 0.8445 | −0.2135 | 0.9235 | 0.7315 | 0.6995 | −0.2015 | 0.8595 |
| TOP2B | 0.569 | 0.26 | 0.653 | −0.023 | 1.112 | 3.158 | 1.896 | 0.602 | 1.367 |
| TOPBP1 | 0.6745 | 0.1005 | 0.3755 | 0.1615 | 0.4005 | 0.2725 | 0.0145 | 0.7265 | −0.3615 |
| TOPK | −1.0765 | −0.1195 | −0.2675 | 0.5735 | 0.5915 | 1.1415 | 0.1095 | −0.9005 | −0.5675 |
| TP53AP1 | 0.9235 | 0.9375 | 1.6225 | −1.0015 | 0.3235 | −1.1775 | 0.0285 | −0.3195 | −0.4435 |
| TP53BP2 | 0.1935 | 0.4235 | −0.8945 | 2.2075 | 0.0035 | 0.8215 | −4.9515 | −1.5275 | −0.9715 |
| TP53INP1 | −0.5475 | −0.1315 | 0.4745 | 0.1595 | −0.1135 | 1.0895 | 0.7415 | −0.3385 | −0.3665 |
| TPD52L1 | −0.768 | −1.978 | 1.096 | −0.379 | −0.312 | −1.908 | −0.465 | −1.921 | −1.036 |
| TPRT | 0.5055 | 1.0615 | 0.5445 | 0.0095 | 0.3485 | −0.2335 | −0.1025 | 0.2235 | 0.3915 |
| TRA@ | −0.317 | −0.257 | 0.196 | 0.072 | −0.358 | 0.214 | −0.144 | 0.003 | 0.593 |
| TRAF4 | 4.956 | 0.519 | 0.857 | 0.417 | −0.204 | −0.588 | 1.061 | 0.267 | 3.577 |
| TRAPPC4 | 0.006 | 0.785 | −0.463 | −0.545 | 0.183 | 1.71 | 0.214 | −0.442 | −0.252 |
| TRH | 0.1605 | −1.4475 | −2.2585 | −1.7055 | −0.4945 | −1.4825 | 0.4765 | 0.6045 | −0.0715 |
| TRIM17 | 0.3265 | −0.7435 | −0.5845 | −0.1965 | 0.6125 | −0.6865 | 1.6845 | 0.3105 | −0.8655 |
| TRIM2 | 0.1425 | −0.0165 | 1.1255 | 0.5405 | 0.1935 | 1.1925 | 0.6485 | 0.3605 | 0.4555 |
| TRIM6 | −0.155 | −0.443 | 0.507 | −0.162 | 0.498 | 0.678 | 0.753 | 1.308 | 0.807 |
| TRIP3 | 0.9025 | −0.1155 | 0.5685 | −0.0885 | 0.0125 | 0.1275 | 0.5305 | 1.0225 | 0.9945 |
| TRPS1 | 1.0125 | 0.7455 | −0.1575 | 0.2735 | −0.1095 | 0.6075 | 0.3785 | −0.0095 | 0.5685 |
| TSG101 | −0.0165 | 0.0225 | −0.6085 | −0.7835 | −0.2665 | 0.8925 | 0.6815 | 0.5375 | 1.1495 |
| TTC10 | −0.5825 | −0.4505 | −0.1485 | −0.1695 | 0.5885 | 0.9885 | 0.0385 | 0.2905 | 0.0405 |
| TTC12 | −0.232 | −0.335 | −0.357 | −0.282 | −0.288 | −0.548 | −0.217 | −0.392 | −0.01 |
| TTK | −0.2915 | −0.7225 | −0.7305 | −1.0705 | −0.9755 | −1.4455 | −1.8515 | −0.3545 | −0.8185 |
| TTLL4 | −0.211 | −0.157 | −1.423 | −0.553 | 0.068 | −1.448 | −0.515 | −0.415 | 1.26 |
| TTYH1 | 1.4405 | −0.4195 | 0.2485 | −0.0225 | 0.1175 | −0.7975 | −1.0715 | −0.3555 | 0.1755 |
| TUBB3 | 1.1975 | −0.0155 | −0.3965 | −0.2145 | 0.5655 | 0.0865 | 0.9515 | −0.4885 | −0.0295 |
| TULP3 | 0.215 | 0.225 | −0.215 | 0.785 | 1.475 | 1.832 | 2.189 | 0.895 | 0.771 |
| TXN | 0.648 | 0.473 | 1.335 | 0.258 | 0.159 | 0.184 | −0.22 | 0.334 | −0.086 |
| TYMS | −2.2105 | −0.7615 | −2.8325 | −1.0725 | 0.8255 | −0.8775 | −0.6305 | −1.8665 | −1.3285 |
| U5-116KD | 0.6345 | −0.0395 | −0.1795 | 0.1935 | 0.0805 | 0.9545 | 1.0075 | 0.3695 | 0.0125 |
| UBE2E3 | −0.071 | 0.081 | 0.354 | 0.092 | −0.104 | 0.075 | −0.335 | 0.156 | 0.12 |
| UBL5 | 0.4085 | −0.3995 | −1.0225 | 0.0555 | −0.5345 | −0.7065 | −2.8015 | −0.7655 | −0.2595 |
| UBN1 | −0.237 | −0.07 | −0.573 | −1.041 | −0.624 | 0.279 | 0.571 | −0.206 | 0.776 |
| UCHL1 | 4.169 | 2.542 | 0.456 | 2.092 | 2.969 | 1.56 | 4.168 | −0.003 | 2.394 |
| UCHL3 | −0.682 | −0.163 | −0.182 | 0.83 | −0.142 | 0.1 | −1.098 | −1.005 | −0.545 |
| UGCG | −0.7745 | 0.0365 | −1.7015 | 0.8025 | −0.2505 | 4.0885 | −0.5305 | 0.7735 | −0.6415 |
| UGCGL1 | −0.4455 | −0.1755 | −0.2045 | −0.2865 | −0.9105 | −1.4785 | −1.7135 | −1.1095 | −0.7295 |
| UGT2B7 | 0.4515 | 0.8615 | 0.6335 | 0.2695 | 0.0535 | −0.8355 | 0.1425 | −0.0535 | −0.1465 |
| UNC13D | −0.213 | −0.232 | −1.04 | 1.049 | 0.414 | 1.083 | 0.584 | 0.231 | 0.781 |
| VAMP8 | −0.86 | −0.244 | 0.418 | 0.075 | 0.256 | −0.804 | −0.953 | −0.159 | −0.186 |
| VAPA | −2.0445 | 1.0245 | 0.8555 | 0.9385 | 2.0485 | −0.2175 | 0.3345 | 0.5575 | 0.4105 |
| VAV1 | −0.37 | −0.308 | −0.288 | 0.67 | 0.028 | −1.621 | −0.836 | −0.878 | 0.28 |
| VAV3 | −0.126 | 0.348 | 0.047 | 0.66 | 0.01 | 0.63 | 0.636 | 0.287 | 0.489 |
| VCX3 | 0.033 | 0.009 | 0.321 | 0.004 | −0.141 | 0.282 | 0.081 | −0.146 | −0.28 |
| VDAC2 | −0.3915 | 0.1285 | −0.4715 | −0.4015 | −0.3015 | 0.0116 | −0.0965 | −0.0915 | 0.1995 |
| VEST1 | 0 | 0.179 | −0.749 | −0.271 | −0.062 | −0.635 | −0.196 | 0.208 | 0.224 |
| VGLL1 | 0.1145 | −0.3415 | −0.1595 | −0.1175 | −0.0795 | −0.2075 | 0.0435 | −0.3265 | −0.4695 |
| VMD2L2 | −0.455 | 0.179 | 0.036 | −0.324 | −0.433 | −0.248 | −0.063 | −0.387 | 0.107 |
| VSX1 | −0.768 | 0.594 | 0.228 | −0.357 | 0.151 | 0.694 | 0.066 | −0.611 | 0.087 |
| WARP | −0.525 | −0.797 | 0.299 | 0.35 | 0.013 | −0.055 | −0.312 | 0.001 | 0.032 |
| WDR26 | 2.0145 | 0.1945 | 0.8055 | −1.1895 | 1.1495 | −0.2415 | 1.9935 | 1.5365 | 2.3585 |
| WDR6 | 2.173 | −0.569 | 0.914 | 0.47 | −0.205 | 1.762 | 1.623 | 0.975 | 0.249 |
| WHSC1L1 | −0.749 | −0.229 | −0.169 | −0.479 | −0.529 | −0.665 | −0.393 | −0.169 | −0.258 |
| WNT3 | −1.5845 | 0.2085 | −0.3315 | −0.1005 | −0.3815 | −0.0195 | 0.2535 | 0.1055 | 0.5305 |
| WNT6 | 2.1105 | 1.9085 | 2.6925 | −0.2655 | 1.2585 | −0.2995 | 1.0435 | 1.3485 | 0.1195 |
| WSB2 | −0.3115 | −0.1575 | 0.0605 | 0.0325 | −0.2825 | −0.1045 | −0.5515 | −0.0975 | 0.0315 |
| WWP1 | −0.1095 | 0.7105 | 0.7395 | 0.5025 | 0.9405 | 0.4745 | 0.3415 | 1.1675 | −1.3405 |
| WWP2 | 0.173 | −0.055 | 0.888 | 0.432 | 0.145 | 0.345 | −0.01 | −0.307 | −0.115 |
| XBP1 | −0.481 | 0.231 | 0.902 | 0.373 | 0.332 | 1.539 | 0.97 | 0.616 | 0.941 |
| XYLT2 | 0.9845 | 0.4865 | 0.3285 | −0.4965 | −0.5095 | 1.5695 | 1.4365 | −0.4735 | 1.8075 |
| YBX2 | −0.167 | 0.264 | 0.352 | 0.631 | 1.28 | 0.884 | 0.435 | 0.573 | −0.178 |
| YIF1 | 0.0545 | 0.9885 | 0.6095 | 0.8935 | −1.8765 | −0.3695 | 0.4585 | 1.1595 | 0.3505 |
| ZA20D3 | 0.0965 | −0.9025 | −0.2095 | 0.4145 | −0.1385 | 0.4105 | −0.3425 | 1.2015 | 0.2225 |
| ZAP128 | 0.4015 | 0.0895 | −0.1925 | 0.6745 | 0.0225 | −0.4015 | −0.3235 | 1.1095 | 0.4705 |
| ZBED3 | −0.88 | 0.267 | −0.301 | 0.212 | 0.056 | −0.353 | −0.039 | 0.582 | −0.613 |
| ZBTB4 | −0.101 | −0.207 | −0.641 | 0.444 | 0.182 | 0.726 | −0.005 | 0.994 | 0.186 |
| ZCWCC1 | 1.3755 | −0.2875 | 0.9165 | −0.0895 | 0.6395 | −0.9385 | 1.5475 | 1.1165 | 1.5105 |
| ZCWCC2 | 0.789 | −0.679 | −0.52 | 0.895 | −0.861 | −1.364 | −2.519 | −1.162 | −0.066 |
| ZDHHC13 | −0.4525 | −1.2545 | −1.1125 | −0.5905 | 0.8985 | −1.2905 | −1.9315 | −0.1005 | −0.6015 |
| ZDHHC14 | −0.6165 | −0.4185 | 0.2085 | 0.2465 | −0.4675 | 0.1245 | 0.0715 | −0.0955 | −0.2585 |
| ZFHX4 | −0.7455 | 0.3405 | −0.3325 | 0.0895 | 0.2075 | −0.3335 | −0.3645 | 0.4585 | 0.4685 |
| ZFYVE21 | 0.156 | 0.747 | 0.858 | 0.339 | −0.771 | 1.219 | 0.252 | 0.331 | −0.43 |
| ZNF133 | −0.427 | 0.745 | 0.089 | 0.197 | 0.036 | −0.331 | 0.537 | 0.483 | 0.76 |
| ZNF165 | −0.4565 | −0.4935 | −0.5635 | 0.0845 | −0.2125 | 0.1985 | 0.2975 | −0.1575 | 0.2735 |
| ZNF24 | 0.238 | −0.109 | 0.616 | 0.683 | 0.477 | 0.533 | 0.002 | −0.431 | 0.089 |
| ZNF318 | −0.2595 | −0.1465 | −0.0055 | −0.0215 | −0.3475 | −0.5045 | 0.1305 | −0.1755 | −0.0845 |
| ZNF54 | −2.8185 | −1.6365 | −1.5575 | −2.1015 | −1.0705 | −0.5775 | −1.8215 | −0.5375 | 0.9275 |
| ZNF432 | −0.4615 | −0.3435 | −1.1105 | −0.1145 | 0.0655 | −0.3365 | 0.7875 | −1.0525 | −0.0705 |
| ZNF482 | 2.583 | 2.234 | 1.079 | 0.485 | 0.902 | −0.61 | −0.116 | 1.887 | 0.619 |
| ZNF505 | 0.891 | −0.189 | −0.878 | 0.871 | −0.492 | −0.541 | 0.576 | −0.026 | 0.559 |
| ZNF552 | 2.8925 | 2.7465 | 0.6375 | −0.2785 | 0.9885 | −0.9235 | −1.0975 | 1.6285 | 1.1265 |
| ZNF577 | −0.4495 | 0.0395 | −0.2415 | −0.0065 | 0.0925 | 0.0115 | −0.3065 | 0.0765 | 0.0485 |
| ZNF587 | 0.0165 | 0.0605 | 0.3385 | 0.4695 | −0.2985 | −0.1515 | 0.7185 | 0.5665 | −0.2185 |
| ZNF598 | 0.8685 | 0.7575 | −1.4315 | 0.0545 | −0.2285 | 0.0045 | 1.1195 | −0.0125 | 0.5975 |
| ZNF599 | −0.043 | −0.242 | −0.541 | 0.221 | −0.133 | −0.047 | 0.272 | −0.083 | 0.492 |
| ZNF606 | 0.105 | 0.586 | 0.329 | 0.199 | 0.448 | 0.884 | 1.161 | 0.325 | −0.123 |
| ZNF614 | 0.373 | 1.226 | 1.745 | −0.547 | 0.829 | 0.3 | 0.951 | −0.196 | 0.344 |
| ZNF639 | | | | | | | | | |
| ZNF7 | | | | | | | | | |

| | Sample | | | | | | | |
|---|---|---|---|---|---|---|---|---|
| | UB-88 | 030311-M [symbol] | BR95-015 [symbol] | 9830-0002 | 9830-0005 | PB307 | BR98-016 [symbol] | UB78 |
| | Class | | | | | | | |
| Symbol | LUMINAL | LUMINAL | LUMINAL | LUMINAL | LUMINAL | LUMINAL | LUMINAL | LUMINAL |
| | −1.5785 | 0.3745 | 1.1915 | 0.7745 | 2.2795 | 3.5635 | −0.5615 | 0.8325 |
| | 0.103 | 2.893 | −0.276 | 1.094 | 2.215 | 1.441 | 2.29 | 0.09 |
| ABAT | 1.513 | 1.816 | 2.231 | 0.46 | 1.09 | −0.921 | 0.755 | −1.328 |
| ABC1 | 1.9455 | 0.8805 | 1.4835 | 0.3625 | −1.3755 | 0.0265 | −0.4455 | 0.3785 |
| ABCC11 | 1.0205 | 1.0755 | 0.1495 | −0.6135 | 0.6895 | 0.6145 | 1.8315 | 0.1075 |
| ABCC3 | −0.443 | −0.928 | −0.729 | 0.173 | −0.794 | −0.536 | −0.756 | −0.489 |
| ACAS2L | −0.2905 | 0.4765 | 0.4765 | −0.0535 | 0.0965 | −0.1745 | 0.0935 | 0.4405 |
| ACAT1 | 3.167 | −1.856 | −1.061 | 3.401 | 3.683 | 2.804 | −0.368 | −0.997 |
| ACAT2 | −0.5065 | −0.1035 | 0.1905 | 0.8055 | −0.4455 | 0.4295 | −0.1355 | −0.0295 |
| ACOX2 | −1.0025 | 0.9065 | 0.9165 | −0.2325 | 0.0985 | 0.4375 | 1.2045 | 0.3045 |
| ACDX3 | 1.4285 | 2.1555 | 0.7675 | 0.0165 | −1.3045 | 0.9155 | −0.2945 | −0.3255 |
| ACSL3 | 0.438 | 0.079 | −0.084 | 0.303 | −0.34 | −0.634 | −0.373 | −0.644 |
| ACTA1 | −0.1325 | 0.3155 | 0.1885 | −0.0095 | 0.1985 | 0.1105 | 0.2975 | 0.0115 |
| ACTB | 1.8955 | 1.4065 | −0.5415 | 3.2335 | 2.2365 | 0.3795 | 0.4685 | −0.4835 |
| ADAM33 | 0.11 | −1.424 | −0.769 | −1.203 | −1.826 | −1.121 | −0.916 | −0.535 |
| ADAMDE [symbol] | −0.3025 | 2.2075 | 0.6525 | 1.9735 | −1.2845 | 0.8055 | −0.6025 | 0.7615 |
| ADPN | 1.1985 | 3.3575 | 0.0775 | 0.3385 | −0.2935 | 0.7835 | 2.8835 | 1.1085 |
| ADPRHL1 | 1.5485 | 2.3175 | −0.2355 | 2.8475 | 2.3825 | 1.8965 | 1.1975 | 0.9085 |
| ADRA2C | 1.3145 | 0.3875 | −0.4195 | 0.7345 | −0.9095 | −0.3625 | −0.7835 | −0.5745 |
| AGR2 | 0.0725 | 0.2065 | −0.1755 | 0.3605 | 0.6175 | 1.4465 | −0.3565 | 0.2375 |
| AGT | −0.403 | −0.33 | 6.173 | −0.568 | −0.379 | 0.069 | 0.085 | −0.114 |
| AGTR1 | −0.116 | 2.66 | 0.979 | 0.508 | −1.448 | 1.126 | 4.423 | 0.408 |
| AHCYL1 | 0.576 | −0.442 | −1.153 | 0.112 | 0.519 | 0.278 | −1.209 | 0.201 |
| AHNAK | −0.08 | 1.355 | 2.465 | −0.753 | 0.451 | −1.187 | −1.313 | −0.177 |
| AHR | −0.2305 | −0.1885 | 0.2645 | −0.3285 | −0.2345 | −0.0985 | −0.0435 | −0.0515 |
| AIM1 | −0.2105 | −0.4675 | −0.6305 | −0.1385 | 0.4745 | 0.2135 | −0.8405 | −0.0125 |
| AK2 | −0.7085 | −1.7745 | −1.0265 | −0.4525 | −2.5995 | −1.3865 | −1.5775 | 0.5825 |
| AK3L1 | 0.401 | −0.525 | −0.853 | −0.368 | 0.883 | 0.064 | 0.041 | −0.372 |
| AK5 | −0.393 | −0.524 | 0.261 | 0.169 | 0.199 | −0.192 | 0.032 | −0.325 |
| AKAP1 | −0.5485 | 0.3525 | 0.4405 | 1.8885 | 0.5265 | 0.9285 | 0.7525 | 0.6495 |
| AKR1A1 | −0.592 | 0.756 | −0.793 | 0.308 | −0.338 | 0.635 | 0.384 | −0.5 |
| AKR7A3 | 0.6395 | −0.0515 | 0.0975 | −0.2915 | −0.0955 | 0.6375 | −0.6945 | 3.2215 |
| ALG5 | −1.1015 | 1.5645 | 0.0725 | 1.3605 | 0.6225 | 0.8575 | 1.5995 | −0.0655 |
| ALG8 | 0.2185 | −0.4935 | −0.3265 | −0.5235 | −0.9315 | −0.5195 | −0.1355 | −0.6245 |
| AMFR | 1.04 | 0.723 | 0.024 | 0.105 | 0.633 | −0.779 | −1.095 | 1.034 |
| AMPD2 | −0.163 | 1.568 | 1.62 | 0.202 | 0.447 | −0.044 | 1.29 | 0.036 |
| ANAPC11 | 0.382 | −0.124 | 0.133 | −0.279 | 0.54 | 0.237 | 0.529 | 0.26 |
| ANKRD22 | −0.33 | 0.928 | 1.821 | 0.164 | 1.127 | 0.066 | 0.245 | 0.903 |
| ANKRD27 | −0.321 | −0.078 | −0.484 | 0.167 | −0.058 | 0.359 | 0.697 | −0.068 |
| ANLN | −4.437 | −1.187 | 0.312 | −1.268 | −3.159 | −1.414 | −3.234 | −1.063 |
| ANP32A | 1.079 | 0.287 | −0.138 | 1.399 | 0.621 | 0.282 | −0.865 | 0.404 |
| ANXA3 | 0.7665 | 1.6585 | 1.9275 | 2.1075 | −0.9265 | 1.0095 | 0.0665 | −1.9325 |
| APBB2 | −1.2785 | 1.3085 | −0.9925 | −0.6276 | −1.5035 | −1.0655 | −1.2075 | 0.3335 |
| APOBEC3 | 0.175 | 0.817 | 2.197 | −0.793 | 1.188 | 0.502 | −0.032 | 0.941 |
| AQP5 | 3.226 | 1.812 | 0.921 | 4.22 | 0.087 | 3.697 | −1.519 | 1.924 |
| AR | −0.2305 | 0.4245 | −0.5435 | 0.5575 | −0.5545 | 0.5045 | 0.6815 | 0.2045 |
| AREG | 0.882 | −0.508 | −0.288 | −0.095 | −0.144 | −0.138 | −0.589 | −0.276 |
| ARF1 | −0.0225 | 0.1985 | −0.0545 | −0.3685 | −0.1645 | 0.7835 | 0.8585 | 0.6775 |
| ARHGDI [symbol] | −0.741 | −0.004 | −0.02 | −0.84 | −0.73 | −0.151 | 0.322 | −0.259 |
| ARL6IP | −0.4445 | −0.9445 | −0.2745 | 0.4815 | 1.1105 | −0.0845 | −0.1555 | −1.1625 |
| ARL6IP2 | 0.643 | 1.003 | 1.002 | −1.421 | −3.437 | −2.255 | 0.504 | 0.581 |
| ARMCX2 | 0.664 | 0.977 | 0.784 | −1.271 | −3.308 | −2.504 | 0.711 | 0.571 |
| ARNT2 | −0.216 | −1.04 | −0.471 | −1.523 | −0.758 | −0.965 | −0.649 | −0.426 |
| ARNT2 | −0.0895 | −0.8295 | −0.4405 | 0.6035 | −0.7905 | −0.6515 | −0.1975 | −0.1105 |
| ARP3BET [symbol] | 0.0095 | −0.1705 | 1.2355 | 0.0415 | −0.1595 | 0.2635 | 0.2545 | 0.1555 |
| ARPC1B | 3.594 | 2.793 | 0.482 | −0.881 | −0.759 | −0.251 | −0.199 | 0.334 |
| ARPC5L | −0.9225 | −0.3925 | −0.1135 | −0.2176 | 0.3085 | −0.9095 | −0.2405 | −0.3035 |
| AS89 | 0.331 | −0.115 | −0.202 | −0.114 | −0.252 | −0.133 | 0.39 | 0.545 |
| ASF1A | −1.4345 | 0.8825 | 0.2825 | −0.2735 | 1.2175 | 0.1535 | −0.2015 | 0.8125 |
| ASH2L | 0.064 | 6.579 | 1.183 | −0.242 | 1.418 | 0.688 | 1.119 | 0.521 |
| ASK | −1.102 | −1.191 | −0.244 | −0.25 | −1.434 | −0.909 | −0.854 | −1.032 |
| ASPM | −0.2255 | −0.1555 | 1.2245 | 0.4405 | 0.9045 | −0.0835 | 1.0905 | 1.2445 |
| ASS | 0.2795 | 1.4265 | −0.3155 | −0.1235 | −0.1265 | −0.3325 | −0.3055 | 0.7735 |
| ATAD2 | −0.0765 | −0.1355 | −2.0735 | −0.8785 | −0.4555 | −0.8015 | 0.2305 | −0.5885 |
| ATF5 | 0.261 | 0.928 | −0.154 | 0.321 | 0.122 | −0.585 | −0.394 | 0.145 |
| ATF7IP2 | 0.452 | −0.038 | 2.241 | −0.101 | −1.223 | −0.017 | −0.448 | 0.54 |
| ATP2B1 | −0.129 | −0.025 | −0.174 | −0.423 | −0.192 | −0.466 | −0.523 | −0.022 |
| ATP5G1 | 0.4895 | 1.0535 | −0.0695 | 0.4715 | −0.3285 | 0.2885 | 1.4795 | 0.8575 |
| ATP5O | −1.146 | −0.098 | −0.552 | −1.305 | 0.068 | −0.344 | 2.376 | 2.22 |
| ATP6AP1 | 0.181 | −1.071 | 0.32 | −0.809 | −0.992 | −0.582 | −0.954 | 0.313 |
| ATP6V0A4 | 0.071 | −0.323 | 0.46 | −0.003 | 0.575 | 1.056 | 0.731 | 0.738 |
| ATP6V1B [symbol] | −0.7375 | −0.0035 | −0.4855 | −0.7545 | −0.2025 | 0.3765 | 0.8995 | −0.4825 |
| ATP6V1G [symbol] | −0.09 | 0.015 | 0.063 | −0.28 | −0.816 | −0.196 | 0.116 | −0.295 |
| ATPIF1 | −0.7165 | 0.0875 | −1.0135 | −0.2075 | −0.6435 | −0.7505 | −1.3825 | −0.2585 |
| ATXN10 | 0.352 | −0.216 | −0.32 | −0.539 | −1.627 | −1.237 | −0.645 | −1.195 |
| B3GNT5 | −0.7395 | −0.3835 | −0.3755 | −0.7425 | 0.1375 | 1.5416 | 0.7245 | 1.5685 |
| BACE2 | −0.0265 | −0.4895 | −0.1676 | 0.0895 | 0.3195 | 0.3525 | 0.4245 | −0.1635 |
| BAG1 | −0.5805 | −0.5945 | −0.0835 | 0.2295 | −1.1585 | 0.3805 | −0.1745 | 0.4515 |
| BAG4 | −3.1875 | −0.1915 | 0.1155 | −1.5205 | −0.6826 | −2.1025 | 2.4195 | −1.0365 |
| BAI2 | 0.436 | 0.816 | 1.578 | 2.009 | −0.763 | 0.394 | 2.198 | 1.067 |
| BAMBI | −0.334 | −0.297 | −0.101 | 0.251 | 1.732 | 0.066 | 0.202 | −0.115 |
| BCAS1 | 1.615 | 1.046 | −4.14 | 2.494 | 2.127 | 2.106 | 1.751 | 0.578 |
| BCL2L14 | 3.2115 | 1.3595 | 0.0405 | −0.1595 | −0.3745 | −0.5265 | −0.1705 | −0.0125 |
| BCMP11 | 1.269 | 1.546 | 0.012 | −1.395 | −1.207 | −0.908 | 1.058 | −1.209 |
| BEX1 | 2.0525 | 2.1315 | −0.6775 | −2.5435 | −1.1615 | −0.0045 | −3.5105 | −1.2655 |
| BEX2 | 1.3075 | 2.9495 | −0.1035 | 0.1785 | 0.0315 | −0.8055 | −1.4135 | 1.3035 |
| BF | −0.238 | 0.606 | 0.077 | 1.032 | 0.253 | 0.472 | 0.57 | 0.111 |
| BFSP1 | 0.066 | −0.253 | −0.643 | −0.892 | −0.234 | −0.581 | −0.243 | −0.469 |
| BIK | 0.782 | 1.563 | 0.488 | 0.506 | 1.072 | −0.474 | 1.004 | 1.391 |
| BIRC2 | 0.5985 | 0.9805 | −0.0345 | 0.6125 | 1.4605 | 1.8335 | 1.3585 | −0.3265 |
| BIRC5 | −1.6545 | 0.2135 | −0.2875 | 0.0235 | −0.9085 | −1.0955 | −0.1075 | −0.6055 |
| Bit1 | 0.8895 | 0.7145 | −0.1655 | 0.1575 | 1.3155 | 1.5625 | −0.8885 | 0.3845 |
| BIVM | 0.427 | 0.465 | 0.182 | 0.641 | 0.948 | 0.038 | 0.65 | −0.433 |
| BLVRA | 0.073 | 3.398 | −0.43 | −0.157 | −1.387 | −1.291 | −1.594 | −0.214 |
| BM039 | −0.364 | −0.222 | −0.678 | −0.072 | −0.42 | −0.616 | −1.225 | 0.144 |
| BMP7 | 0.673 | −0.014 | 0.636 | −0.655 | 0.084 | −1.149 | −0.524 | 1.658 |
| BOC | 0.8945 | −0.0415 | −0.0395 | −0.0225 | 0.3665 | 0.2065 | 2.8785 | 0.5735 |
| BRCA1 | 0.0795 | −0.4795 | −0.5555 | 0.2785 | 0.1545 | 0.2095 | −0.5395 | −0.3645 |
| BRF2 | 0.325 | 2.431 | −0.241 | 0.331 | −0.046 | −0.713 | −0.3 | 1.347 |
| BRI3 | −1.158 | 0.243 | −1.138 | −0.121 | −0.114 | −0.762 | −0.429 | −0.587 |
| BRI3BP | 0.838 | −0.483 | −0.784 | −0.521 | 0.118 | 0.556 | 0.38 | 0.401 |
| BSPRY | −0.0555 | −0.4535 | −0.0095 | 0.3585 | −1.7195 | −0.9625 | −0.6885 | 0.0875 |
| BTF3 | 0.2455 | 0.0216 | 0.0016 | −0.2675 | 0.5725 | 0.0275 | 0.2205 | 0.2755 |
| BTG3 | −0.127 | 0.458 | 0.898 | 0.823 | 1.205 | 0.201 | 0.328 | 0.89 |
| BγN2A2 | 1.388 | −0.238 | 1.286 | 0.849 | 2.38 | 1.157 | 1.398 | −0.452 |
| BUB1 | −0.3335 | −1.3025 | 1.7625 | −0.1005 | −0.2285 | −0.7015 | −0.4995 | −0.5575 |
| BUB3 | −0.166 | −0.654 | −0.51 | 0.583 | −0.993 | −0.56 | 0.319 | −0.328 |
| BXDC1 | −0.22 | −0.144 | 0.873 | 0.461 | −0.187 | 0.594 | 1.108 | 0.094 |
| BZRP | −0.477 | 0.817 | 0.59 | 0.231 | 1.508 | 0.076 | 0.413 | 0.7 |
| BZW1 | −0.3225 | −0.7695 | 0.0395 | −0.3215 | −0.6245 | −0.5885 | −1.0365 | −0.4805 |
| C10orf116 | 0.5005 | 0.8205 | −0.3005 | −0.8855 | −0.1575 | −0.1045 | −0.3305 | 0.0325 |
| C10orf38 | −0.222 | 0.034 | 0.042 | −0.004 | 0.695 | −0.594 | −0.607 | −0.454 |
| C10orf4 | −6.0135 | 0.5705 | −0.1255 | 0.1475 | −0.2535 | −0.0315 | −0.2295 | 0.0135 |
| C10orf45 | −0.7125 | 3.6075 | 0.2755 | −1.0715 | 1.0165 | −0.2635 | 3.1685 | 0.0245 |
| C10orf7 | 0.0675 | 0.7505 | −0.2895 | 0.0345 | −0.7465 | −0.1625 | −0.3145 | −0.7035 |
| C10orf82 | 0.79 | 0.457 | −0.338 | 0.106 | −0.208 | −0.901 | −0.834 | −0.531 |
| C11orf24 | −1.1485 | −0.9155 | −0.7745 | −0.8525 | −0.8735 | −0.3015 | 1.2255 | 0.1985 |
| C12orf14 | −0.533 | 1.407 | −0.06 | 0.033 | −0.397 | 0.358 | −0.112 | −0.262 |
| C13orf21 | 0.086 | −0.335 | −0.399 | 0.281 | 0.322 | 0.247 | 0.144 | −0.033 |
| C13orf23 | −0.361 | −0.025 | 0.216 | −0.387 | 0.497 | 0.884 | 0.53 | 0.333 |
| C14orf94 | 2.8 | 0.168 | −0.273 | 1.22 | 0.27 | −0.92 | −0.96 | −0.298 |
| C15orf23 | 0.5985 | 3.1915 | 2.0175 | 1.0075 | 1.5785 | 0.6575 | −0.3365 | 0.7005 |
| C16orf45 | 1.549 | 0.133 | 0.71 | 0.212 | −0.903 | 0.015 | 1.014 | 0.48 |
| C17orf37 | 0.433 | 0.681 | −0.578 | 0.406 | −0.345 | −0.002 | −0.214 | −0.041 |
| C18orf1 | −1.824 | −0.263 | −0.451 | 0.809 | 0.329 | −0.116 | 0.843 | −1.064 |
| C19orf10 | 1.5895 | 0.3965 | 0.4245 | −0.1465 | 0.6155 | 1.2715 | 1.4895 | 1.2445 |
| C19orf32 | −1.1765 | −0.2895 | −0.3225 | 0.2615 | 1.8405 | −0.3025 | −1.4595 | −0.7415 |
| C19orf33 | −0.215 | −0.424 | 0.523 | 0.416 | 2.019 | 1.538 | 1.356 | −0.021 |
| C1orf24 | 0.294 | 0.904 | −0.242 | 0.492 | −0.426 | −0.315 | −1.112 | 0.591 |
| C1orf34 | 0.1735 | 0.4625 | 0.7795 | 0.1955 | 0.6335 | 0.6985 | 0.3265 | 1.0585 |
| C20orf103 | 0.6215 | 1.4035 | −1.4835 | 0.7945 | 1.9775 | 2.7775 | −1.0915 | 1.4205 |
| C20orf149 | 0.371 | 0.704 | 0.298 | −0.742 | −0.267 | 0.765 | 2.042 | −0.359 |
| C20orf23 | 0.824 | −0.045 | −1.185 | 0.095 | 0.514 | 0.223 | 0.701 | 0.119 |
| C20orf3 | 0.602 | −0.426 | 0.061 | −0.315 | 0.331 | 0.019 | 0.364 | −0.122 |
| C20orf35 | 0.66 | 0.343 | −1.252 | −0.154 | 0.329 | −0.132 | −1.039 | 0.795 |
| C20orf43 | −0.0065 | 0.2265 | −0.0765 | −0.1415 | 0.1295 | −0.1685 | 0.1175 | 0.5525 |
| C20orf8 | 0.528 | 1.016 | 0.683 | 0.623 | 0.007 | 0.351 | 0.881 | 0.17 |
| C21orf45 | 0.866 | −0.255 | −0.576 | −0.808 | −0.796 | 0.142 | −0.14 | −0.765 |
| C22orf18 | 0.02 | −0.416 | −0.182 | −1.02 | −0.514 | 0.407 | 0.389 | 0.135 |
| C2F | 0.501 | 1.421 | −0.346 | 0.319 | −0.636 | −1.016 | −1.019 | −0.462 |
| C2orf17 | 0.474 | 0.303 | 0.535 | 0.318 | −0.878 | −0.717 | −1.261 | 0.048 |
| C4A | 0.509 | 0.317 | −0.363 | −0.226 | −0.783 | −0.016 | −0.001 | 0.453 |
| C5orf13 | 0.209 | 0.23 | −0.921 | −0.387 | 0.257 | −0.493 | 0.347 | −0.104 |
| C6orf108 | −0.192 | −1.59 | −1.012 | −0.764 | −0.658 | −0.757 | −0.843 | −0.438 |
| C6orf166 | 1.072 | 1.377 | 2.328 | −0.188 | 2.118 | 3.319 | 1.125 | 0.399 |
| C6orf192 | −0.0325 | −0.1325 | −0.1525 | −0.4885 | −0.0535 | 0.1655 | −0.2515 | 0.3435 |
| C6orf211 | 0.582 | 0.827 | −0.033 | −1.536 | −0.467 | −0.925 | 0.298 | −0.524 |
| C6orf49 | −0.5755 | 0.1275 | 1.9265 | 0.2855 | −0.9915 | −0.3745 | −0.2405 | −0.2115 |
| C6orf85 | 0.8165 | 0.0905 | 0.2525 | 0.6465 | 0.5005 | −0.2255 | −0.0645 | 0.4815 |
| C7orf24 | 1.657 | −1.65 | −0.34 | −0.521 | 0.767 | 0.605 | −0.47 | 0.014 |
| C9orf10 | −0.848 | −1.731 | −0.287 | −1.012 | −0.401 | 0.706 | 1.636 | 1.401 |
| C9orf121 | 0.102 | 0.369 | 0.306 | 0.412 | −0.48 | 0.401 | 0.745 | 0.241 |
| C9orf58 | −0.3705 | −0.6435 | −0.2805 | −0.2455 | −0.8745 | −0.8885 | −0.3595 | −0.3285 |
| C9orf88 | 0.385 | −0.363 | −0.341 | 0.331 | 0.279 | −0.82 | 1.353 | 0.035 |
| C9orf9 | 0.575 | 0.501 | −0.241 | −1.094 | −2.14 | 0.574 | −0.248 | −1.135 |
| CA12 | 0.689 | 0.083 | −0.617 | −1.731 | −2.512 | 0.421 | −0.476 | −1.051 |
| CA8 | −0.228 | −0.092 | 0.067 | 0.37 | 0.013 | −0.255 | −0.332 | 0.048 |
| CA8 | 0.0065 | 1.3335 | −0.2875 | 0.1845 | −0.3065 | 0.0125 | 0.1076 | 0.4405 |
| CACNA1G | −0.414 | −0.518 | −0.221 | 0.895 | 0.394 | −0.036 | 0.423 | −0.149 |
| CALCA | −0.8 | −0.651 | −0.94 | 0.878 | 0.193 | 0.439 | −0.198 | −0.219 |
| CALML4 | −0.1215 | 0.0785 | 0.1435 | −0.2555 | −0.4885 | −0.3025 | 1.7335 | 0.4385 |
| CALML5 | 2.4225 | 0.4485 | 0.4315 | −1.3055 | −0.9615 | 0.8505 | −0.4435 | 0.6965 |
| CaMKIINa [symbol] | 0.481 | −0.012 | −0.448 | −0.001 | 0.364 | 0.095 | −0.034 | 0.372 |
| CAP2 | 0.688 | 0.724 | −0.671 | 0.199 | −0.086 | −1.034 | 0.42 | −1.08 |
| CAP350 | −1.3455 | −1.3395 | −0.3695 | −0.7755 | −1.0585 | 0.9335 | −0.1845 | 1.4695 |
| CAPN13 | 0.176 | 0.389 | −0.089 | −0.734 | −0.629 | 0.026 | 2.913 | −0.662 |
| CAPN9 | 0.4005 | 0.2015 | −0.3225 | 0.1205 | 0.4625 | 0.1875 | −0.2215 | 0.3965 |
| CARD14 | −0.0985 | −0.7025 | −0.6625 | −0.2755 | 0.2315 | −0.0205 | −0.3495 | 0.1295 |
| CART | −0.1395 | 0.4905 | 0.4485 | 0.2176 | 0.3815 | 0.2165 | 0.2115 | 0.8305 |
| CASP1 | −0.554 | −0.228 | −0.535 | 0.26 | −0.769 | 0.31 | −0.787 | −0.781 |
| CASP3 | −0.171 | −0.114 | −0.238 | 0.301 | 0.372 | 0.188 | 0.178 | −0.52 |
| CASP7 | −1.0255 | −1.7305 | −1.6295 | 0.2645 | 0.8645 | 0.1845 | −1.8055 | 0.0275 |
| C9LN1 | −1.076 | −0.106 | 0.578 | 0.031 | 0.481 | −1.014 | −1.95 | 0.861 |
| CBR1 | 0.2575 | −0.8235 | 1.3855 | 0.2825 | 0.1285 | 0.0105 | −0.1815 | −0.2285 |
| CBR3 | 0.15 | 0.172 | −0.029 | 0.144 | −0.078 | −0.037 | −0.067 | −0.039 |
| CBR4 | −1.3416 | −0.5385 | 0.2585 | −0.8635 | −1.2616 | −0.3535 | −1.4945 | 1.5555 |
| CCL24 | −0.599 | 0.259 | 0.774 | 0.281 | 0.976 | 0.334 | 0.652 | 0.837 |
| CCL29 | 1.301 | 0.819 | −0.188 | 1.858 | 3.044 | 0.289 | 1.572 | 3.255 |
| CCNB2 | 0.0375 | 1.1595 | 0.5825 | 0.6655 | −0.0655 | −0.1945 | 0.8135 | 0.2825 |
| CCND1 | 0.26 | 0.271 | 0.831 | −0.371 | 0.972 | 1.249 | 0.552 | −0.477 |
| CCNE1 | 0.6645 | −0.0585 | 0.1355 | 0.1435 | −0.3415 | 0.4635 | −0.0915 | −0.0005 |
| CCNG2 | −0.424 | 0.246 | 0.123 | 1.217 | 2.508 | 0.146 | 1.461 | 0.159 |
| CCNH | −0.1395 | 0.3195 | 0.3145 | 1.1805 | 1.8585 | 1.2395 | 1.0895 | 0.7516 |
| CD38 | −0.2335 | 0.4885 | −0.1635 | 0.2975 | 0.9015 | 0.1175 | 0.9885 | 0.0505 |
| C030 | −0.1135 | −0.2635 | −0.3765 | 0.1255 | −0.2475 | −0.0705 | −0.6525 | −0.0625 |
| CD4 | −0.3565 | −1.5715 | 0.1175 | −1.3215 | −0.7955 | −1.3025 | −1.2555 | −0.4965 |
| CD58 | −0.316 | 0.472 | 2.438 | 1.753 | 1.266 | −0.005 | 0.128 | 1.005 |
| CD50 | 0.3045 | −0.0105 | 0.1145 | 0.7775 | 2.5745 | 0.0305 | −0.3305 | 1.1285 |
| CDC2 | −0.2675 | 1.1125 | 0.3055 | 0.4075 | 1.6265 | 0.6925 | −0.4205 | 0.8715 |
| CDC6 | 0.6325 | 0.3235 | 1.0955 | −0.1995 | 0.4615 | 0.1535 | 0.9076 | 2.0035 |
| CDCA1 | −1.539 | −0.658 | 1.249 | −1.411 | 0.08 | 0.153 | 1.314 | −0.265 |
| CDCA5 | 0.109 | 1.438 | 0.846 | 0.988 | 0.969 | 1.11 | 0.996 | 1.947 |
| CDCA7 | −0.8585 | 0.9135 | −0.4915 | −0.5745 | −0.4245 | −0.5815 | −0.7065 | −0.2915 |
| CDH1 | 0.806 | 0.695 | 0.167 | −0.838 | 0.565 | −0.431 | −0.448 | 1.05 |
| CDH3 | 0.0945 | 0.2385 | 0.6665 | 0.4745 | 0.0645 | −0.1915 | −0.4935 | −0.0155 |
| CDK2 | 0.5405 | 1.4885 | −0.4505 | 0.0005 | 0.0135 | 0.8545 | 1.2985 | 1.1655 |
| CDKN2A | −0.1925 | 0.9825 | 0.3685 | 1.3705 | 1.2345 | 0.2245 | 0.5745 | 0.3365 |
| CDKN2D | 0.286 | 1.427 | 0.839 | 0.481 | 0.434 | 0.528 | 0.015 | 0.536 |
| CDKN3 | −0.08 | 0.291 | 0.139 | −0.525 | −0.564 | −0.148 | 0.38 | 0.306 |
| CDT1 | 1.3295 | −0.1465 | −0.6185 | 1.7505 | −0.0135 | 1.4855 | 1.0425 | 1.0315 |
| CE9PG | −0.519 | 0.158 | 0.716 | 0.242 | 0.568 | 0.168 | 0.649 | 0.168 |
| CELSR1 | 0.178 | 0.394 | 0.954 | 0.127 | 0.738 | 0.128 | 0.64 | 0.874 |
| CENPA | 0.213 | 0.034 | 0.601 | −0.651 | 0.427 | 0.196 | 0.675 | 0.461 |
| CENPE | −0.322 | 0.172 | −0.3 | −0.711 | −0.433 | −0.185 | 0.139 | 0.336 |
| CENPF | −0.0455 | 0.1895 | 0.2945 | 0.0555 | 0.3155 | 0.1765 | 0.8895 | 0.0845 |
| CENTG3 | 0.5165 | −0.4135 | −0.5895 | 0.6385 | 1.2275 | 0.1805 | −0.3005 | 0.5225 |
| CES2 | 0.274 | 0.857 | 0.408 | −0.105 | 1.48 | −0.323 | 5.884 | −0.163 |
| CETN3 | −1.1125 | 0.1185 | −0.3605 | −0.4295 | −0.7625 | −0.4195 | −0.8135 | −0.1315 |
| CGA | 0.8195 | 1.0935 | 2.5575 | 1.8005 | −0.6985 | 1.6065 | 0.8395 | 0.2715 |
| CGI-12 | 1.0595 | 1.4075 | −1.3375 | 0.2445 | 0.8575 | 0.8565 | 0.5685 | 0.5885 |
| CGI-49 | 1.093 | 0.883 | −0.775 | 0.372 | −0.547 | 1.043 | 1.325 | −1.589 |
| CGI-4 [symbol] | −3.273 | −0.294 | 1.426 | −0.761 | −1.491 | −1.373 | −2.772 | 0.194 |
| CGN | −0.312 | 0.803 | 0.243 | −0.347 | 1.458 | 0.104 | −0.133 | 0.943 |
| CGNL1 | −0.3855 | 0.3435 | −1.3285 | 0.5485 | −0.0615 | −0.3116 | −1.0955 | 0.7805 |
| CHAF1B | 0.39 | −0.016 | −1.385 | 0.519 | −0.263 | −0.183 | 0.78 | 0.026 |
| CHC1 | 0.441 | 0.074 | 0.282 | 0.438 | 0.839 | 0.495 | 0.082 | 0.517 |
| CHCHD2 | 2.097 | −0.409 | −1.013 | −1.165 | 0.89 | −0.11 | −0.018 | −0.08 |
| CHCH05 | −2.07 | −1.644 | −1.739 | −2.028 | −2.063 | −1.648 | −1.696 | −0.727 |
| CHI3L1 | 0.168 | 0.632 | −0.925 | −1.053 | −1.178 | −0.796 | −0.439 | −0.845 |
| CHI3L2 | −0.203 | −0.073 | −0.013 | −0.149 | −0.169 | −0.165 | 0.081 | −0.227 |
| CHKA | −0.344 | −0.27 | −0.217 | 0.037 | −0.293 | −0.009 | 0.083 | 0.025 |
| CHCDL | 0.0135 | 0.3075 | 0.1285 | 0.1515 | 0.0935 | 0.2045 | −0.0985 | −0.0955 |
| CHRDL2 | 0.736 | 0.271 | −0.564 | 0.435 | 1.177 | 0.348 | −0.146 | 0.159 |
| CHST2 | −0.0835 | −0.5205 | −0.1765 | 0.0145 | 0.4315 | 0.3925 | −0.0975 | −0.0165 |
| CHURC1 | 0.218 | 0.101 | 0.124 | −0.177 | 0.002 | 0.888 | 0.348 | −0.385 |
| CIR | 0.1765 | 0.0805 | −0.0475 | 0.2745 | 0.4655 | −0.1955 | 0.0055 | 0.3375 |
| CIRBP | −0.1425 | 0.0375 | −0.6315 | −0.0515 | 0.7275 | 0.5895 | −0.3105 | −0.3385 |
| CKLF | 0.0245 | −1.0835 | −1.2585 | −0.4125 | −0.5195 | −1.0005 | −1.0695 | 0.0535 |
| CKLFSF6 | −2.0325 | −0.1305 | 0.8285 | −1.0415 | −0.4175 | −0.5395 | −1.6135 | 0.6915 |
| CKLFSF7 | 0.149 | −0.005 | −0.145 | −0.063 | 0.647 | 0.265 | −0.35 | 0.858 |
| CKMT1 | 0.015 | 0.283 | 0.371 | 0.017 | 0.702 | −0.015 | 0.132 | 0.732 |
| CKS1B | 0.359 | −0.17 | 0.502 | 0.599 | 0.679 | −0.163 | 0.159 | −0.102 |
| CKS2 | 0.3075 | 0.4045 | −0.7985 | 0.4215 | −1.2685 | 0.4055 | −1.0445 | −0.0245 |
| CL640 | −1.2795 | 1.8055 | −0.5865 | 2.5495 | 0.7315 | 0.8255 | −0.0025 | 1.0485 |
| CLDN23 | −1.2775 | 0.6365 | 0.4195 | −0.8925 | −1.0125 | 0.2725 | −0.2405 | 0.2585 |
| CLDN3 | 0.1885 | −0.8215 | −0.3215 | −0.8915 | −0.8065 | −1.3355 | −1.1425 | 0.1285 |
| CLDN4 | 0.227 | 2.403 | −2.089 | −0.279 | 2.988 | 2.223 | −1.075 | 0.45 |
| CLDN8 | −0.2535 | −0.7325 | −0.7445 | 1.1025 | −0.4115 | 0.1065 | −0.8565 | −0.1805 |
| CLGN | 0.7195 | −0.7235 | −0.0005 | −1.1055 | −0.6075 | −0.2545 | −0.5235 | 3.0135 |
| CLIC6 | 0.505 | 1.115 | 0.483 | 1.313 | 2.594 | 2.772 | 2.841 | −0.113 |
| CLNS1A | −1.5785 | −0.0505 | −0.5585 | −0.3935 | 1.8615 | −0.5775 | 0.6955 | −0.5135 |
| CLTC | −0.009 | −0.313 | 0.268 | −0.591 | −0.55 | −0.417 | −0.218 | 0.342 |
| CLU | −0.596 | 0.21 | 0.28 | −0.041 | −0.241 | −0.551 | 0.368 | 0.34 |
| CMAS | −0.1975 | 0.2735 | −0.7955 | 0.4955 | 0.7385 | −0.1715 | 0.2135 | −0.1146 |
| CML65 | −0.309 | 0.194 | −0.361 | −0.308 | 0.201 | −0.26 | −0.302 | 0.585 |
| CNIH | 0.038 | 1.079 | −0.034 | 0.619 | −0.071 | −0.161 | −0.136 | 0.353 |
| CNOT10 | 0.3205 | −0.0965 | −0.6895 | −0.4305 | 0.5695 | 0.6385 | 0.0335 | −0.3685 |
| CNOT2 | 0.168 | −0.805 | −0.678 | 0.489 | −0.859 | −0.248 | −0.261 | −0.378 |
| CNOT7 | −1.187 | 2.149 | 4.287 | −1.751 | 1.853 | −2.525 | 1.077 | 3.214 |
| CNP | 0.7475 | 0.2455 | −0.0515 | −0.1285 | −0.8085 | −0.4835 | −0.3665 | 0.5145 |
| CNTNAP2 | −0.255 | −0.291 | 1.044 | 0.188 | 0.767 | 0.002 | 0.599 | 0.285 |
| COASY | −0.8175 | −1.7255 | −0.5715 | −1.2065 | −1.6035 | −1.0575 | 0.4375 | 0.0055 |
| COL11A2 | 1.472 | 0.351 | 1.925 | −1.182 | 0.27 | −0.24 | −1.903 | −0.269 |
| COL27A1 | −0.157 | 0.726 | 0.26 | −0.656 | −0.253 | 0.667 | 0.507 | 0.033 |
| COL4A5 | −1.6695 | −1.4255 | −0.8215 | −0.6515 | −1.8315 | −0.9235 | 0.7375 | 0.1685 |
| COL8A1 | −0.313 | −0.927 | −0.393 | 0.473 | 0.443 | 0.195 | 0.449 | 0.453 |
| COL8A3 | 0.17 | 0.695 | −0.218 | 0.23 | −0.414 | −0.31 | −0.316 | 0.554 |
| COMMD1 [symbol] | 0.1065 | 0.1115 | 0.3415 | −0.0765 | −0.7655 | −0.1535 | 0.1415 | 0.1305 |
| COMMD5 | 0.0755 | 0.1765 | −0.0915 | −0.2825 | −0.2755 | −0.2405 | −0.0515 | −0.9695 |
| COPS7B | −0.295 | 0.259 | −0.027 | 0.609 | 0.808 | −0.158 | 0.301 | 0.565 |
| CORO6 | 0.3345 | 0.8555 | 0.6135 | 0.6565 | 0.9185 | 0.7755 | 0.4565 | −0.0825 |
| CORT | −0.383 | 0.012 | −0.117 | 0.169 | −0.186 | 0.088 | 0.768 | 0.267 |
| COX17 | −0.281 | 0.031 | −0.041 | 0.534 | −0.06 | 0.331 | 0.98 | 0.147 |
| COX5A | 0.977 | 0.864 | 0.109 | 0.527 | 0.844 | 0.824 | 1.037 | 0.471 |
| COX6A | 1.2905 | −0.1375 | −1.2175 | 0.8735 | 1.6805 | 2.9655 | 2.2195 | 1.2245 |
| COX8A1 | 0.0165 | 0.2045 | −0.5355 | −0.1126 | 0.1465 | −0.3185 | 0.6145 | 0.9245 |
| COX8C | 1.8695 | −0.5295 | −1.0715 | −0.8015 | −1.2355 | −0.8705 | −1.4735 | −0.6475 |
| COX7A2 | −0.988 | 0.819 | −0.838 | −1.431 | 0.214 | −0.738 | −0.707 | −0.067 |
| CP | −0.3775 | 0.0655 | −0.3715 | 1.5845 | −0.0875 | 3.0785 | 3.2395 | 2.3405 |
| CPAMD0 | 0.298 | −0.212 | 0.043 | −0.165 | 0.724 | 0.472 | 0.024 | 0.848 |
| CPB1 | 0.5545 | 0.0785 | −0.8045 | 0.0645 | 0.3715 | −0.0545 | 1.7335 | −0.8345 |
| CPEB3 | 0.051 | 0.475 | 0.142 | 0.206 | −0.113 | −0.213 | −0.373 | 0.311 |
| CPEB4 | −0.237 | 0.129 | 0.12 | 1.134 | 0.042 | −0.33 | −0.268 | −0.5 |
| CPSF3 | −0.8855 | 0.6755 | 0.3255 | 0.0685 | 0.4085 | −0.2765 | −1.0535 | 1.0405 |
| CRABP1 | 0.863 | −0.059 | −2.661 | 0.104 | 1.391 | 0.816 | 0.937 | 0.693 |
| CREB3L4 | −0.084 | 0.373 | 0.111 | 1.141 | −0.333 | 0.031 | 1.288 | −0.061 |
| CRIP1 | −0.22 | 0.889 | −0.433 | 0.831 | −0.513 | −0.084 | 0.557 | 0.016 |
| CRYBB2 | −1.657 | −0.704 | −0.382 | −0.77 | 0.371 | −0.415 | −0.183 | −0.089 |
| CRYL1 | −0.229 | 0.177 | 0.269 | −0.015 | −1.172 | −0.757 | −0.873 | −0.597 |
| CRYZ | 0.4085 | 0.3735 | 0.5825 | −0.3105 | 0.2405 | 0.4915 | 0.2125 | 0.4165 |
| CSDA | −0.2265 | 0.1225 | 0.0745 | 0.3295 | 0.1695 | −0.2175 | 0.5145 | 0.0055 |
| CSE1L | −1.0505 | 0.1255 | 0.9685 | −0.8905 | −1.3935 | 0.1995 | −0.8675 | 0.0235 |
| CSK | −0.9765 | −0.0625 | −0.7245 | −0.7795 | 0.5375 | −0.1265 | −1.6635 | −0.5985 |
| CSRP1 | 1.0925 | 0.1205 | −1.3395 | 0.3865 | 1.9025 | −0.5975 | 1.7575 | −1.4075 |
| CSRP2 | 0.762 | −0.081 | −0.42 | 0.927 | −0.008 | −0.149 | −0.646 | −0.125 |
| CST5 | −1.1755 | −0.2185 | 0.0705 | −0.4505 | −0.5455 | 0.1875 | −0.5955 | 0.6395 |
| CSTB | −0.369 | 0.236 | −0.629 | −0.687 | −0.014 | 0.002 | −0.445 | −0.278 |
| CTAG1B | 0.7585 | 0.6625 | 0.5455 | 0.5525 | −0.1245 | 0.1345 | 0.0865 | 0.1335 |
| CTBP2 | −0.102 | −0.322 | 0.385 | −0.069 | −0.396 | −0.182 | −1.107 | 0.085 |
| CTNNA1 | 1.255 | 2.042 | 0.107 | 1.562 | 1.258 | 1.292 | 0.812 | 1.216 |
| CTNNAL1 | −0.2935 | −0.0245 | 0.1255 | −0.3815 | −0.1775 | −0.7915 | 0.1925 | 0.3445 |
| CTNND2 | 1.346 | 0.385 | 0.489 | 0.379 | 0.215 | −0.002 | 0.186 | −0.127 |
| CTPS | 1.096 | −0.239 | −0.057 | 1.159 | 1.268 | 0.469 | 0.826 | −0.114 |
| CTSL2 | 0.5675 | 2.3325 | 0.1985 | 0.8595 | 0.2325 | −0.3285 | 0.4655 | 0.1625 |
| CTSS | −2.77 | −0.85 | −1.855 | −1.24 | −0.68 | −0.55 | −2.18 | 0.997 |
| CTTN | 1.3235 | −0.2945 | 1.7515 | 1.9375 | 3.1885 | 1.2595 | 0.8965 | 1.4415 |
| CX3CL1 | 0.2875 | 0.7385 | 0.0555 | 0.9785 | 0.1495 | 0.5005 | 0.0215 | 0.0395 |
| CXCL9 | 1.4705 | −0.9045 | −0.1185 | 0.8085 | −0.4955 | 1.1855 | 0.4955 | −0.4805 |
| CXorf39 | 0.009 | −1.766 | −1.262 | 0.121 | −1.588 | −0.998 | −1.076 | −0.315 |
| CXXC4 | 0.265 | 0.265 | 0.602 | 1.542 | 0.324 | 0.647 | 1.506 | −0.322 |
| CYB5 | −0.1985 | 0.1085 | −0.5925 | 0.9435 | 0.0465 | 0.1115 | 0.0685 | 0.8165 |
| CYB561 | 0.069 | −0.054 | −0.446 | −0.518 | −0.213 | −0.411 | −0.463 | 0.412 |
| CYC1 | 0.748 | −0.021 | 0.126 | 0.533 | −0.101 | −0.084 | 0.093 | −0.262 |
| CYHR1 | −1.5305 | −0.5816 | −1.2385 | −1.4136 | −0.4255 | 0.1705 | −1.4315 | −0.1385 |
| CYP2A13 | −0.362 | 0.198 | −0.003 | −0.381 | −0.921 | −0.402 | 0.39 | −0.434 |
| CYP4X1 | 0.9745 | 1.4395 | 1.0585 | 0.6615 | 2.3045 | 1.1295 | 1.5435 | 1.2175 |
| D15Wsu7 [symbol] | 0.0675 | 1.8776 | 0.1785 | −0.5175 | 1.0645 | 1.1265 | 2.1725 | 1.9545 |
| DACH1 | 0.994 | 1.78 | 1.36 | 0.627 | 2.905 | 1.638 | 1.911 | 1.41 |
| DACH1 | 0.838 | −0.2 | −0.19 | −0.285 | −0.365 | 0.582 | −0.148 | −0.251 |
| DACH1 | −0.513 | −0.889 | 0.001 | −0.098 | 0.702 | −0.594 | −0.083 | −0.88 |
| DAP3 | 0.2195 | 0.1405 | −0.0955 | −0.4755 | 0.7615 | 0.7465 | 0.2845 | −0.1745 |
| DAPP1 | −0.347 | −0.882 | 0.104 | −0.486 | −0.532 | −0.583 | −0.859 | −0.349 |
| DAZAP2 | 0.601 | 1.073 | 0.885 | −0.657 | −2.042 | 0.04 | 0.059 | −0.542 |
| DBI | −0.0085 | −0.3975 | 0.4565 | 0.6615 | 0.4685 | 0.0945 | 0.5475 | −0.0195 |
| D9N1 | 0.496 | −0.032 | −0.783 | −0.048 | −0.754 | −0.896 | −0.681 | 0.288 |
| DC13 | −0.0216 | −0.5195 | 0.0225 | −0.2085 | −0.4835 | −0.0755 | 0.2445 | 0.1195 |
| DCPS | 0.4345 | −0.6285 | 0.1665 | 0.6565 | −0.4465 | −1.0775 | 0.4805 | −0.4855 |
| DDHD2 | 0.3985 | 0.7265 | 0.4945 | 0.4725 | 0.7725 | −0.0385 | 0.6015 | 0.4015 |
| D0X17 | 0.8655 | −0.0845 | 0.5275 | −1.1255 | −0.0465 | −0.3835 | 2.4795 | 0.8815 |
| DDX39 | −0.666 | 0.042 | −1.084 | 0.814 | 1.362 | 0.95 | −0.676 | −0.533 |
| DDX48 | 0.2255 | −0.3015 | −0.7695 | 0.0925 | 0.7165 | 0.5355 | 0.5545 | 0.3885 |
| DDX58 | 0.1375 | −0.1455 | 0.1195 | −0.8695 | 0.8205 | 0.4355 | −0.8705 | −0.1345 |
| DEF6 | 0.697 | 0.692 | 0.595 | −0.006 | −1.004 | −0.48 | −0.594 | 1.835 |
| DEK | −0.181 | 0.717 | −0.318 | 0.378 | 0.889 | 0.966 | −0.037 | 0.219 |
| DHCR7 | −1.118 | −0.989 | −0.592 | −0.802 | −1.57 | −0.762 | −1.025 | −1.175 |
| DHRS7 | −0.8925 | −1.3015 | 0.3915 | −0.4825 | −1.3405 | −1.2755 | −0.2895 | 0.1875 |
| DHX57 | −1.4745 | −0.4175 | 1.7095 | 2.4525 | 1.0325 | −0.8825 | −1.7325 | 0.8135 |
| DIRC1 | −0.144 | −0.075 | −0.87 | 0.191 | 0.305 | −0.118 | 0.396 | −0.047 |
| dJ222E13 | 0.99 | −0.032 | 0.128 | −0.678 | 0.512 | −0.075 | −0.437 | −0.571 |
| DKFZP434 | −0.566 | 1.1 | −0.455 | −0.122 | −0.126 | 0.006 | 0.354 | 0.837 |
| DKFZp434 | 0.1355 | −0.3235 | 0.2335 | −0.0265 | −0.1745 | −0.2675 | 0.3625 | 0.0345 |
| DKFZP434 | 0.339 | −0.476 | 0.451 | 0.405 | 0.301 | 0.611 | 0.139 | −0.012 |
| DKFZp434 | 0.8135 | 0.3375 | −0.0825 | −0.0515 | −0.1355 | 0.3625 | 0.1045 | 0.1555 |
| DKFZP564 | 0.6045 | 0.1445 | −0.3525 | −0.7505 | −0.1565 | −0.3505 | −1.0565 | 0.0545 |
| DKFZp564 | −0.472 | −0.256 | 0.747 | −0.076 | −0.206 | −0.586 | 1.254 | −0.278 |
| DKFZP564 | −0.0995 | 0.2375 | 0.1215 | 0.2425 | 0.0425 | 0.1505 | 0.4355 | 0.1275 |
| DKFZP564 | 0.64 | −0.476 | 0.087 | −0.197 | −0.468 | −0.133 | −0.559 | 0.483 |
| DKFZP564 | −1.682 | 0.284 | −1.008 | −1.493 | −1.865 | −0.132 | −1.508 | −0.229 |
| DKFZp586 | −0.1045 | 7.3525 | 0.3965 | 0.8395 | −0.6405 | 0.9115 | −0.4295 | 1.2585 |
| DKFZp761 | 0.005 | 0.17 | 0.002 | −0.006 | −0.299 | −0.032 | 0.322 | −0.075 |
| DLK1 | −1.812 | 1.064 | 0.4 | 0.925 | 1.352 | 1.325 | 3.022 | 1.367 |
| DLX5 | 0.0995 | 1.7985 | 0.1025 | 1.9025 | 0.1485 | 0.9015 | −0.4695 | 0.3145 |
| DNAJA4 | 1.7195 | 3.0375 | −1.5865 | 1.9985 | 1.8835 | 3.3705 | 1.6505 | 1.4835 |
| DNAJC1 | −0.587 | 0.056 | 1.889 | 1.836 | −0.366 | −1.331 | −0.264 | 0.502 |
| DNAJC12 | 1.471 | 0.142 | −3.143 | 1.204 | 1.572 | 1.191 | 0.827 | 0.041 |
| DNAJD1 | 0.223 | −0.182 | −0.182 | −0.224 | 0.329 | −0.399 | 0.437 | 0.221 |
| DNALI1 | −0.214 | −0.157 | −0.177 | −0.939 | 1.249 | 1.889 | 1.322 | 0.585 |
| DNMT1 | −1.0745 | −0.2345 | −0.4585 | −1.1235 | −1.2775 | −0.8475 | −1.4045 | 0.1015 |
| DOX1 | 0.2735 | −0.1365 | −0.8045 | −0.0075 | −0.0085 | 1.0925 | 0.0545 | −0.7215 |
| DSCR1 | 0.3045 | 0.5345 | −0.7125 | −1.3275 | −1.8895 | −2.5235 | −0.1455 | −1.4925 |
| DSCR1L2 | −0.1525 | −0.1385 | 0.4345 | 0.0635 | −0.3295 | 0.1635 | 0.2595 | 0.8145 |
| DSP | 0.6005 | 0.1805 | −0.9555 | −0.2865 | −0.3525 | 0.5895 | −1.2815 | −0.0285 |
| DTYMK | 0.2405 | 0.9635 | −0.0235 | 0.0545 | −0.4235 | −0.8875 | −1.1545 | −0.2435 |
| DUSP23 | −0.5515 | 1.8705 | 0.4535 | −1.3765 | −1.1675 | −0.3305 | −1.6875 | 0.1855 |
| DUSP4 | −0.8025 | 0.0965 | −0.1395 | −0.0635 | −0.7495 | 0.0435 | 1.0695 | −0.5625 |
| DUSP5 | −0.053 | 0.438 | 0.13 | 0.828 | 0.663 | 0.41 | 0.511 | 0.204 |
| DVL1 | 0.388 | −1.059 | −0.244 | 0.493 | 0.45 | −0.376 | −0.229 | −0.232 |
| DXYS155 [symbol] | −0.31 | 0.048 | 0.292 | −0.192 | −0.271 | −0.814 | 0.246 | 0.298 |
| DYRK4 | −1.1645 | 1.3795 | 2.6716 | 0.0315 | 1.7855 | 0.2445 | 0.7745 | 0.2555 |
| o(y)2 | 0.979 | 2.685 | 2.665 | 1.283 | −0.349 | 1.018 | 0.034 | 0.341 |
| EAF2 | −0.1545 | 1.0475 | −1.9345 | −0.6855 | −0.7895 | −0.2185 | −0.2015 | −0.0975 |
| EAP30 | −0.9085 | 0.0935 | −1.7495 | −0.2465 | −0.4495 | −0.9455 | −0.2375 | −0.3285 |
| ECHDC1 | −0.2075 | 1.9835 | 2.8425 | 2.1525 | 0.5876 | 0.7445 | 0.7025 | 1.6195 |
| ECHDC3 | 0.7745 | −0.0655 | 0.4215 | −1.0245 | 0.9665 | 1.5125 | 0.1785 | −0.4505 |
| EEF1A2 | −1.061 | 0.292 | −0.902 | −0.464 | 0.025 | −1.177 | −1.638 | −0.404 |
| EFHC1 | −2.1485 | 0.1325 | 0.2665 | −0.5835 | −1.0205 | −2.5745 | 0.1585 | −0.6816 |
| EFNA4 | −0.5245 | 0.5725 | 0.2445 | 0.5935 | −0.1155 | −0.0005 | −0.0525 | 0.1885 |
| EFS | −0.3165 | −0.6905 | −0.1195 | −0.2815 | −0.2705 | 0.8395 | 0.0645 | −0.0985 |
| EIF2C2 | −0.2005 | −0.7225 | −0.0095 | 0.1895 | −0.8985 | −0.5355 | 0.6605 | −0.5115 |
| EIF2C4 | 0.287 | 0.002 | 0.473 | −0.111 | −0.738 | −0.087 | 0.246 | −0.163 |
| EIF4EBP1 | 0.1675 | −0.4465 | −0.0075 | 0.0085 | −0.1845 | −0.2405 | 0.3855 | −0.2085 |
| EIF4G1 | −0.71 | −1.655 | −1.171 | −1.028 | −1.052 | −0.533 | −0.754 | −0.414 |
| EIF4G3 | 1.838 | 0.029 | −1.351 | 0.019 | −0.794 | −0.742 | −0.540 | −1.069 |
| ELAC1 | −0.266 | 0.334 | 0.423 | −0.741 | 1.565 | 0.244 | 0.328 | 0.87 |
| ELL2 | 0.1335 | −0.5975 | −0.0945 | −0.7495 | 0.8515 | −0.3035 | −1.5885 | −0.2835 |
| ELL3 | −0.1465 | 0.0935 | −0.2575 | −0.2125 | −0.6505 | −1.3285 | −0.5805 | −0.2665 |
| ELOVL5 | −0.6025 | 0.4855 | −0.0385 | −0.1435 | −0.4705 | 0.4965 | 0.2825 | −0.5835 |
| EN1 | −0.2345 | −2.9135 | −2.6505 | −1.7655 | 0.4125 | −1.5555 | 0.1445 | −0.0466 |
| ENAH | −0.1875 | 0.2715 | 0.1765 | −0.3755 | 0.2285 | 0.4165 | 0.2345 | −0.5855 |
| EPHX2 | 0.86 | 2.607 | 2.971 | 2.887 | −1.575 | 1.838 | 0.396 | 1.438 |
| EPLIN | 0.1445 | 0.0455 | −0.4805 | −0.7265 | 0.6776 | 0.1905 | −0.3125 | 0.1055 |
| EPN3 | 0.4615 | 4.3665 | 3.0085 | 2.4905 | 2.0075 | 0.0245 | −1.5815 | 0.9425 |
| EPOR | −0.429 | 0.707 | −0.936 | −0.682 | 0.009 | −0.299 | −0.721 | −0.173 |
| ERBB2 | 0.2775 | −0.4055 | 1.8355 | −1.5075 | 1.4395 | 2.0195 | 1.5885 | 2.2646 |
| ERP70 | −0.611 | 0.035 | 0.787 | 0.708 | 0.417 | 0.299 | −0.566 | 0.469 |
| ESR1 | 0.3915 | −0.5725 | −0.8445 | −0.4035 | −0.6755 | −0.5825 | −0.3555 | −0.3935 |
| ETFA | 0.1635 | −0.4055 | 0.3915 | 0.7855 | −0.4325 | −0.3165 | 0.2825 | 1.1785 |
| ETV8 | 0.2655 | 0.5665 | 0.5565 | 0.5155 | 0.5435 | −0.3045 | −0.3515 | 0.8585 |
| EVER1 | 0.1065 | 0.5555 | 0.3245 | 0.3905 | −0.0345 | 0.2765 | −0.2775 | 0.9735 |
| EXO1 | 0.4326 | −0.1955 | −0.6455 | −0.4465 | 0.0575 | −0.0935 | −0.4586 | 0.1545 |
| EXOSC9 | −0.92 | 0.649 | 1.039 | −0.513 | 0.914 | 0.294 | 0.668 | 0.565 |
| EXT2 | 0.051 | 0.204 | −0.638 | −0.288 | 0.671 | 0.604 | −1.158 | −0.004 |
| EZH2 | 0.0145 | −0.0915 | 0.0695 | 0.0875 | 0.0525 | 0.0625 | 0.0565 | 0.0325 |
| FI1R | −0.718 | 2.765 | 1.894 | 0.64 | −1.241 | 0.779 | 2.197 | 2.12 |
| F9 | −1.081 | 1.145 | 0.884 | −1.156 | −2.016 | −0.354 | −0.226 | 0.951 |
| FA2H | 0.028 | 2.424 | −0.235 | 1.005 | 0.984 | 0.015 | 1.331 | 1.39 |
| FABP7 | 0.699 | 0.76 | 0.222 | 0.034 | 1.345 | 1.448 | 0.488 | 0.313 |
| FADD | 0.359 | −0.785 | 0.284 | −0.543 | −0.463 | −0.832 | −0.008 | −0.225 |
| FAHD1 | −0.041 | −0.924 | −0.43 | −0.938 | −1.289 | 0.348 | 0.143 | −0.928 |
| FAHD2A | −0.104 | 0.39 | −0.081 | 0.774 | −0.219 | −0.158 | −0.395 | 0.373 |
| FAM38A | −0.9315 | 4.8855 | 1.7235 | 1.6285 | 3.2965 | −0.9015 | −1.2355 | 4.5935 |
| FAM3A | −0.7505 | −0.7155 | 0.1275 | −0.4255 | −0.8175 | −0.2785 | −0.1645 | 0.4555 |
| FAM3B | 0.033 | 0.906 | −0.07 | −0.114 | 0.868 | −0.565 | 0.495 | 1.117 |
| FAM3C | 0.115 | 0.548 | −0.651 | 0.083 | −0.24 | 0.695 | 0.198 | 0.094 |
| FAM46C | −0.745 | 1.63 | 0.013 | 0.216 | 0.038 | −0.358 | −0.022 | 1.274 |
| FAM49B | −0.488 | −0.073 | 0.577 | 0.175 | 0.553 | 0.362 | 0.112 | 0.637 |
| FAM54A | −0.189 | −0.199 | −0.003 | −0.903 | 0.501 | 0.236 | −0.044 | 0.58 |
| FANCA | 0.4455 | −0.0095 | −0.6205 | −0.4345 | −1.1405 | −0.5975 | −0.8535 | 0.2345 |
| FANCE | 1.3725 | 0.6686 | 1.2016 | 1.0325 | 1.1416 | 1.3545 | 0.9885 | 0.6615 |
| FBL | 0.022 | 0.534 | 0.13 | −0.328 | −0.181 | 0.33 | 0.87 | −0.073 |
| FBP1 | 0.891 | 1.372 | 1.093 | 1.421 | 0.979 | −0.255 | −0.16 | 0.428 |
| FBS1 | 0.3985 | 0.3275 | −0.9645 | −0.8235 | −0.4355 | 0.1625 | 0.1275 | −0.2895 |
| FBXL20 | 0.133 | 1.267 | 0.378 | 0.972 | 0.315 | 0.547 | 1.481 | 0.888 |
| FBXO25 | −0.454 | −0.28 | −0.248 | 0.339 | 0.084 | 0.586 | −0.064 | 0.138 |
| FDXR | 0.02 | 3.33 | 2.198 | 2.923 | 1.15 | −0.499 | 0.191 | 1.013 |
| FEM1B | −1.747 | 1.404 | 1.032 | 2.304 | −1.425 | 0.239 | −1.429 | −0.767 |
| FGF12 | −0.3975 | −0.0995 | 0.2445 | 0.7765 | −0.4025 | −0.2875 | 0.3265 | 0.1995 |
| FGF13 | −0.583 | 0.629 | −0.251 | 0.006 | 0.243 | 0.07 | 0.556 | 0.215 |
| FHOD1 | 0.2355 | −0.1835 | 0.3125 | 1.7485 | −0.9065 | 0.0585 | −0.3725 | −0.3135 |
| FKBP11 | −0.0095 | −1.0305 | 0.6316 | 0.4985 | 1.0535 | 0.5895 | 0.6895 | −0.1915 |
| FLJ10094 | −0.376 | −1.037 | −0.566 | 0.089 | −0.132 | 0.227 | −0.638 | 0.376 |
| FLJ10115 | −0.0495 | −0.0455 | −0.3925 | −0.1995 | −0.5445 | −0.2655 | 0.2495 | 0.9825 |
| FLJ10159 | −0.113 | −0.002 | 0.052 | −0.373 | −0.025 | 0.299 | 0.548 | 0.316 |
| FLJ10204 | −0.17 | 0.758 | −0.125 | −0.05 | 0.686 | 0.17 | −0.303 | 0.953 |
| FLJ10275 | −0.7426 | −1.4735 | −1.1895 | 1.2015 | 0.5485 | 1.2645 | 1.6485 | 1.6495 |
| FLJ10292 | 0.5205 | 1.5005 | 0.6926 | −1.1025 | −0.1395 | 0.3725 | 0.5445 | 0.5035 |
| FLJ10324 | 0.1665 | −0.0125 | 0.3485 | −0.5515 | −0.0945 | −0.4045 | −0.5485 | 0.9955 |
| FLJ10700 | 1.057 | 0.324 | −0.491 | −0.179 | −0.054 | 0.051 | −0.791 | −0.266 |
| FLJ10706 | −2.036 | 1.094 | −0.201 | −0.983 | −1.948 | −1.383 | 0.281 | 0.294 |
| FLJ10671 | −0.505 | 0.439 | −0.691 | −0.258 | −0.656 | 0.665 | 0.527 | −1.927 |
| FLJ10901 | 0.0055 | −0.2115 | 0.5835 | −0.3065 | 2.0315 | 0.8005 | −0.0055 | −0.0305 |
| FLJ10918 | 0.935 | −0.476 | −0.128 | −0.017 | −1.229 | −0.333 | −0.887 | −1.103 |
| FLJ10980 | −0.0555 | 0.4065 | 0.0645 | −0.2745 | 0.1535 | 0.2745 | 0.5675 | 0.3165 |
| FLJ11017 | −0.0665 | −1.1885 | −0.6085 | −0.3395 | −0.1505 | −1.0605 | −1.2965 | −0.3225 |
| FLJ11088 | −0.8025 | −0.3125 | −0.3065 | −0.7035 | −0.3035 | −0.6116 | 0.5665 | −0.6355 |
| FLJ11151 | 0.9765 | −0.2245 | 0.0425 | −0.1075 | −0.3615 | 0.7116 | 0.2965 | 0.8715 |
| FLJ11267 | −0.0625 | 0.2995 | 0.0216 | 0.3175 | 0.2395 | 0.5885 | 0.7425 | −0.1135 |
| FLJ11280 | −0.0785 | 0.1135 | −0.0815 | −0.2165 | −0.2395 | −0.7475 | 0.2955 | −0.3165 |
| FLJ11508 | −1.3335 | 1.4905 | −0.3095 | −0.8135 | 1.0675 | 0.7145 | −0.8045 | 0.3785 |
| FLJ12270 | 3.3855 | −0.1855 | 0.4485 | 0.4165 | 2.2795 | 3.1635 | 3.2385 | 3.7765 |
| FLJ12844 | 0.4235 | 1.7225 | 0.8235 | −0.4295 | −0.2195 | 0.6765 | −0.4455 | 0.8175 |
| FLJ12650 | 0.5385 | −0.7695 | 0.3735 | −0.1215 | 0.4295 | −0.1425 | 0.0115 | 0.5205 |
| FLJ12684 | −0.1305 | 1.0255 | 1.0115 | −0.1295 | −0.6335 | −0.6145 | −0.8265 | −0.0575 |
| FLJ12735 | 0.85 | −0.758 | 0.268 | −1.293 | −1.761 | 0.135 | −2.499 | 0.592 |
| FLJ12750 | 0.541 | −0.736 | −0.051 | −1.853 | 1.683 | 1.574 | 1.826 | 1.969 |
| FLJ12895 | 0.7095 | 3.3595 | 2.7105 | 0.6725 | −0.8055 | 1.3815 | 0.1785 | 0.4905 |
| FLJ13710 | −0.4455 | 0.7195 | 0.0016 | −0.3505 | 0.2635 | 0.3535 | −0.2395 | 1.3335 |
| FLJ13855 | 0.11 | −0.88 | −0.207 | −0.577 | −0.213 | 0.246 | −0.016 | −0.299 |
| FLJ13912 | 1.662 | 1.093 | 0.445 | 0.099 | 0.052 | −0.274 | −0.07 | 0.533 |
| FLJ14124 | −0.444 | 0.853 | 0.336 | −0.767 | −0.227 | 0.248 | 0.436 | 0.238 |
| FLJ14627 | −0.0715 | 0.0995 | 0.0985 | −0.1925 | 2.6395 | 0.1555 | 0.1985 | −0.3675 |
| FLJ14666 | −0.414 | −0.003 | −0.337 | 1.018 | 2.289 | 0.486 | −0.369 | 0.037 |
| FLJ14904 | 2.56 | −0.439 | 0.2 | −0.507 | 2.303 | 3.125 | 1.573 | 1.293 |
| FLJ20035 | 0.299 | 1.78 | −0.564 | 0.254 | −0.086 | 0.356 | −0.47 | 0.3 |
| FLJ20152 | 0.375 | 0.015 | −0.351 | −0.373 | −0.354 | −0.281 | −0.033 | −0.014 |
| FLJ20171 | 0.094 | 0.7 | −0.582 | 0.334 | 0.721 | −0.212 | 0.346 | −0.198 |
| FLJ20244 | −0.9 | 2.495 | 2.258 | 0.764 | 3.755 | 2.39 | 1.497 | 0.937 |
| FLJ20273 | −0.3 | −0.673 | −0.097 | 0.945 | −0.077 | 1.167 | 0.046 | −0.085 |
| FLJ20315 | −0.711 | −0.283 | −0.179 | 0.265 | 0.214 | −0.144 | −0.205 | 0.604 |
| FLJ20323 | −1.0255 | 0.2405 | −0.1535 | 0.5915 | 0.1735 | −0.5855 | −0.2945 | 0.0745 |
| FLJ20518 | −0.427 | −0.216 | −0.818 | −0.215 | 1.036 | 0.757 | −0.228 | 0.201 |
| FLJ20530 | −0.003 | 0.521 | 0.093 | 0.12 | 0.315 | −0.232 | 0.028 | 0.822 |
| FLJ20696 | −0.3005 | −0.2695 | 0.0925 | −0.2885 | 0.1935 | −0.7005 | 0.4205 | 0.4805 |
| FLJ20718 | −0.074 | 0.21 | −0.28 | −0.072 | −0.138 | −0.421 | 0.05 | 0.333 |
| FLJ20772 | 0.8725 | −0.0105 | −0.6075 | −0.7975 | −1.1305 | −0.4325 | −1.1265 | 0.0365 |
| FLJ20989 | −0.0455 | −0.8195 | −0.5205 | −0.2395 | 0.3485 | 0.7035 | −0.1555 | −0.8615 |
| FLJ21019 | 1.0975 | 1.9455 | 0.5195 | 1.3995 | −0.0175 | 0.5845 | 1.0765 | 0.1655 |
| FLJ21062 | 1.1035 | −2.7075 | −0.0195 | −1.0665 | −0.3085 | −0.3975 | −0.1565 | −0.3595 |
| FLJ21159 | −0.4115 | −0.3065 | −0.0475 | −0.1395 | −0.3315 | −0.4425 | −0.5535 | −0.0955 |
| FLJ21616 | 0.008 | −1.978 | 1.819 | −0.415 | 1.009 | −1.452 | 0.387 | 1.111 |
| FLJ21827 | 0.0345 | 0.4885 | −0.1015 | −0.8405 | 0.1105 | 0.1045 | −1.2985 | 0.1145 |
| FLJ21963 | 0.744 | 1.006 | −0.279 | 0 | 0.448 | −0.058 | −0.861 | 0.607 |
| FLJ22104 | 0.6635 | −0.3485 | 0.0195 | 0.6555 | 1.2245 | 0.1945 | 0.2375 | 0.7835 |
| FLJ22222 | 0.414 | 0.479 | 1.369 | −1.139 | 0.574 | 0.443 | −0.694 | 0.616 |
| FLJ22573 | −1.5885 | 2.7345 | 1.3035 | 0.3945 | 2.4225 | 1.9465 | 0.8565 | 0.5805 |
| FLJ22794 | 0.422 | 0.308 | −0.38 | −0.972 | −0.262 | −0.305 | −1.333 | −0.113 |
| FLJ23188 | 0.5745 | 1.1605 | −0.0305 | −0.4905 | 2.1155 | −0.4815 | 0.5335 | 0.2005 |
| FLJ23441 | −0.689 | 1.588 | 1.227 | −0.698 | 0.274 | 0.581 | 0.636 | −0.327 |
| FLJ25471 | 1.591 | 0.055 | −0.466 | −0.074 | −0.203 | −0.815 | −0.839 | 1.03 |
| FLJ31204 | −0.0335 | −0.1025 | 0.3705 | −0.2375 | −0.9615 | 0.0165 | 0.5915 | 0.1035 |
| FLJ31795 | 0.0705 | −0.1255 | −0.7265 | 0.0765 | 0.3975 | 0.0025 | 0.3405 | 0.6385 |
| FLJ32942 | 0.0905 | 0.4825 | 0.4335 | 1.3325 | 3.3755 | 1.4685 | −1.4105 | 0.5195 |
| FLJ37970 | −2.725 | −2.361 | −1.221 | −3.432 | −0.274 | −2.3 | −2.35 | −1.032 |
| FLJ39370 | 0.4045 | 0.1776 | 0.6605 | −0.5865 | 1.3135 | −0.5085 | −0.8815 | 1.2345 |
| FLJ90588 | −0.4745 | −0.7335 | 0.0835 | −0.2465 | −1.0395 | −0.1285 | 0.1945 | −0.1115 |
| FN3KRP | 0.648 | 0.974 | 0.464 | 0.197 | 0.613 | 0.638 | 0.292 | 1.004 |
| FOLR1 | −1.6695 | −0.5055 | −1.0876 | −1.1495 | −0.7595 | −0.2895 | −1.0395 | 0.2195 |
| FOXA1 | 0.4355 | 0.2305 | 0.2385 | 0.5645 | 0.0265 | 0.2765 | −0.5255 | −0.5135 |
| FOXC1 | 0.694 | −0.19 | 0.818 | 0.136 | −2.052 | −0.021 | −0.963 | −0.128 |
| FOXP1 | −0.1305 | −0.1785 | −0.0215 | −0.0485 | 0.2635 | −0.7005 | −0.1155 | −0.2025 |
| FREQ | −0.4425 | −0.1055 | −0.1075 | 0.1035 | 0.4985 | −0.2735 | −0.0045 | 0.5855 |
| FTO | 0.4945 | −0.0075 | −0.7365 | 0.7855 | 0.7075 | 0.4635 | 0.3125 | −0.4835 |
| FUBP1 | 1.3845 | 1.7815 | 0.4005 | 1.7725 | 2.6305 | 1.1985 | 0.6755 | 1.9145 |
| FUCA1 | 0.012 | −0.729 | −1.38 | −0.069 | −0.636 | 0.011 | −0.675 | 0.398 |
| FUT8 | −0.0275 | 0.5445 | 0.5715 | 0.4045 | 0.0805 | 0.6595 | −0.4715 | −0.5445 |
| FXYD6 | −1.155 | −1.114 | 1.249 | −0.768 | −0.939 | −0.423 | −0.855 | 0.293 |
| FYCO1 | −0.35 | −2.389 | −0.916 | −0.574 | −0.778 | −0.698 | −1.957 | 0.213 |
| FZD10 | −0.5715 | 0.3065 | −0.0555 | −0.1185 | −0.6505 | −0.2305 | −0.4665 | −0.3145 |
| FZD7 | 0.0915 | 2.1435 | −0.4195 | 1.1485 | 2.6235 | 0.6855 | −0.9335 | 0.0565 |
| FZD9 | 0.3905 | 0.9885 | −0.2775 | 0.8685 | 2.7425 | 0.3245 | 0.1565 | −0.1455 |
| G1P2 | 0.2105 | −0.4155 | −0.8615 | −0.0535 | −0.0645 | −0.5345 | −0.5025 | 0.0965 |
| G1P3 | −0.131 | −0.295 | 0.234 | 0.141 | 0.174 | 0.493 | 1.058 | −0.242 |
| GA17 | −0.6165 | 1.3885 | 1.3165 | 1.2615 | 1.4545 | −0.2595 | −0.1935 | 1.7655 |
| GABARAP | −0.0075 | 0.2935 | −1.2295 | 1.2965 | −0.0955 | −0.0025 | 0.5585 | −0.8835 |
| GAJ | 1.1235 | 2.5285 | −0.2895 | −0.5825 | −1.0435 | −1.0435 | 2.0025 | −0.5775 |
| GALE | 0.738 | 0.088 | −0.134 | 0.032 | −0.584 | −0.519 | −0.348 | −0.324 |
| GALNT14 | 0.8415 | 0.8485 | 0.3085 | 1.0105 | −0.0275 | 0.8335 | 0.2655 | 0.3555 |
| GALNT2 | −1.5135 | 0.5325 | −0.3155 | 0.9475 | 0.4275 | −0.3595 | 0.4175 | 0.5155 |
| GALNT7 | 0.265 | −0.085 | 1.149 | 0.244 | −0.758 | −0.434 | −0.051 | −1.073 |
| GAMT | −0.336 | 0.264 | 0.483 | 0.259 | −1.199 | −1.01 | −0.579 | −0.202 |
| GAPD | −0.478 | 0.054 | −0.03 | −0.104 | −0.413 | −0.843 | −0.565 | −0.068 |
| GARS | 1.252 | 1.048 | 0.344 | 0.022 | 0.348 | 0.468 | 0.68 | 1.14 |
| GART | 0.2645 | −0.0745 | 0.4655 | 1.0705 | −1.3245 | −0.6245 | 0.4215 | −1.3575 |
| GATA3 | 0.9815 | 0.2735 | −0.4145 | −0.6025 | −0.2445 | 0.1165 | −0.3765 | −0.2585 |
| GATM | 0.895 | −1.625 | −0.767 | −1.049 | −1.583 | −1.521 | 0.758 | −0.443 |
| GBA | −0.442 | −0.212 | −0.209 | −0.613 | −0.641 | −0.31 | −0.311 | 0.106 |
| GDAP1 | 0.248 | 0.744 | 0.172 | −0.278 | −0.89 | 0.024 | 0.404 | −0.556 |
| GDI2 | 0.4805 | 0.4755 | −0.6185 | −0.7285 | 0.3565 | −0.2355 | −0.5555 | 0.7805 |
| GGA1 | 0.3765 | −1.1255 | 0.0735 | 0.3275 | 1.0375 | 0.0995 | 0.0765 | −0.5715 |
| GKAP1 | 0.1465 | 0.2235 | −0.5475 | −0.0985 | −0.3875 | −0.4385 | −0.4305 | −0.3995 |
| GLCCI1 | 0.0945 | −0.3345 | −0.8025 | −0.3445 | −0.7325 | −0.1445 | −0.5235 | 0.1065 |
| GLG1 | −0.007 | −0.909 | −0.03 | −0.049 | −0.196 | −0.245 | −0.342 | −0.596 |
| GLTSCR2 | 0.466 | 0.475 | −0.894 | −0.223 | −1.687 | −0.641 | −0.398 | −1.76 |
| GNB4 | 0.637 | 0.838 | −0.427 | 0.282 | 0.26 | −0.263 | 0.02 | 0.065 |
| GOLPH2 | −0.6795 | 0.4235 | −0.1055 | −0.3345 | −0.2275 | −0.1935 | −0.1965 | 0.1335 |
| GPATC1 | −0.5095 | 0.7255 | −0.4475 | −0.9355 | −3.1205 | −0.3485 | −0.8255 | 0.8095 |
| GPC2 | −0.4505 | 1.5205 | 0.8535 | 1.0835 | 0.8475 | 1.4805 | 1.2705 | 0.2775 |
| GPR125 | −1.582 | 1.89 | 0.124 | 0.412 | −1.411 | −0.075 | 1.247 | 0.13 |
| GPR160 | −0.3005 | 1.5175 | 1.0035 | 0.5515 | −2.4165 | 0.0045 | 0.7365 | 1.2935 |
| GPR56 | 0.5455 | −0.8535 | 0.5895 | −0.0075 | −1.1875 | 0.7305 | 0.0615 | 0.8275 |
| GPRC5C | −0.2225 | −0.0955 | 0.4205 | 0.1075 | 0.6885 | −0.3195 | 0.1165 | 0.8295 |
| GPSM1 | −0.836 | −1.14 | 0.393 | 0.264 | −1.134 | −1.208 | −0.592 | 0.303 |
| GPSM2 | −1.537 | 0.095 | −0.239 | −1.211 | 0.188 | −1.21 | 0.717 | −0.927 |
| GPT | −0.1785 | −1.1875 | −0.9205 | 0.2455 | −1.1395 | −0.7295 | −0.5045 | −0.6195 |
| GPT2 | 1.561 | 2.181 | −0.626 | −0.705 | −1.512 | 0.933 | −0.683 | −1.787 |
| GPX7 | −0.092 | 3.352 | 1.221 | 0.73 | 1.361 | 0.214 | −0.057 | 0.104 |
| GRB14 | 1.2115 | 1.6295 | −1.0575 | 1.5345 | 3.5255 | 1.5595 | −1.3235 | 1.8485 |
| GRB7 | 0.0345 | 2.9005 | 3.1225 | −0.6925 | 1.4485 | −0.5105 | 0.0985 | −0.2435 |
| GSC | 0.191 | −0.223 | 0.417 | 0.38 | 0.42 | 0.102 | −0.067 | 0.013 |
| GSDML | −0.238 | 0.01 | −0.001 | 0.005 | −0.232 | 0.012 | −0.009 | −0.078 |
| GSK3B | −0.839 | −0.859 | 0.078 | −0.833 | 0.203 | −0.571 | −0.612 | 0.011 |
| GSTA1 | −1.7205 | −1.1405 | 3.2075 | −2.0305 | 1.7935 | 0.0085 | −0.1005 | 1.3215 |
| GSTK1 | −0.752 | 2.217 | −2.032 | −1.689 | −1.808 | −1.485 | −2.132 | −1.055 |
| GSTM3 | −8.2445 | 0.8955 | 0.6085 | −1.1825 | −0.3485 | −0.2425 | −2.4815 | 0.9245 |
| GSTP1 | 0.434 | −1.04 | 0.555 | 0.898 | −0.564 | 0.63 | −1.135 | −0.125 |
| GSTT1 | −0.5095 | 0.7855 | −0.2355 | 0.4645 | −0.5605 | 0.0725 | −0.1585 | 0.6355 |
| GSTT2 | −1.1505 | −0.2115 | 0.7055 | 0.2435 | −0.5005 | 0.8175 | 1.2085 | −0.2385 |
| GTF2F2 | −0.54 | 0.344 | 0.109 | −0.242 | −0.227 | 0.776 | 0.714 | −0.265 |
| GTF2I | 0.6715 | 0.1835 | −0.4095 | −0.1045 | 0.3525 | −0.2255 | 0.1545 | 0.2145 |
| GTF3A | −0.032 | 0.288 | −0.189 | 0.279 | 0.019 | 0.264 | 0.052 | 0.019 |
| GTPBP3 | 0.114 | −0.304 | −0.65 | 0.14 | 0.429 | −1.485 | −0.035 | −0.407 |
| GTPBP4 | 0.48 | 0.158 | −0.051 | 0.315 | 0.638 | 0.573 | −0.151 | 0.623 |
| H1F0 | 0.7415 | 0.2295 | 0.5415 | 0.4115 | −0.7385 | −0.0435 | 0.8505 | 0.1445 |
| H2AFV | −0.1015 | 0.4885 | 0.6705 | 0.5045 | 0.8975 | 0.3575 | 0.0155 | 0.8255 |
| H2AFY | −0.0885 | 0.8795 | −0.0265 | 1.0355 | 1.1655 | 1.2615 | 1.1855 | 1.0255 |
| H2AFZ | 0.2855 | 0.3785 | −0.2535 | 0.3885 | 1.1965 | 0.3515 | −0.1535 | −0.0115 |
| H2BFS | 0.7855 | 0.2825 | −0.3765 | −0.1415 | 0.4515 | 0.5645 | 0.0455 | −0.1175 |
| H3F3B | 0.77 | 0.406 | 0.153 | 0.011 | 0.514 | 0.977 | 1.139 | 0.825 |
| H41 | 0.6065 | 0.0145 | 0.1065 | 1.7875 | −0.0955 | 0.1465 | 0.0335 | −0.2545 |
| HAGH | 0.471 | 1.224 | −0.831 | −0.307 | 0.925 | 0.237 | 0.487 | 0.819 |
| HBG1 | −0.1215 | 1.3145 | 1.3095 | 0.3645 | 0.6845 | −0.0855 | 0.6985 | 1.2975 |
| HBL01 | −0.442 | −0.911 | −0.427 | 0.374 | 2.38 | 0.55 | −0.463 | 0.618 |
| HCAP-G | −0.2275 | −0.3675 | −0.3445 | −0.4245 | −0.5775 | −0.3795 | −0.2445 | −0.0035 |
| HCP5 | −0.496 | −0.209 | 0.414 | −0.779 | 2.219 | 0.364 | 0.238 | 0.455 |
| HDAC2 | 0.943 | 0.353 | −0.391 | 0.364 | 2.405 | −0.222 | 3.04 | 1.115 |
| HDC | −0.1015 | 0.9605 | 0.0905 | 0.1295 | 0.9285 | 0.4635 | 0.3565 | −0.0855 |
| HEBP1 | −0.401 | −0.23 | −0.804 | −1.27 | −0.83 | −1.659 | −0.922 | −0.917 |
| HIPK2 | 1.335 | −0.228 | −0.292 | −0.601 | 0.529 | −0.761 | −0.792 | 0.505 |
| HIPK2 | 0.0955 | 0.6515 | −0.0815 | 1.8595 | 1.9135 | 1.8225 | 1.6705 | 0.1455 |
| HIS1 | 0.5775 | 0.7005 | −0.0595 | 1.2445 | 1.6995 | 1.2695 | 1.4975 | 1.1545 |
| HIST1H2A | 0.553 | 0.581 | 0.005 | 1.037 | 1.664 | 1.057 | 1.408 | 1.221 |
| HIST1H2B | 0.5415 | 0.3805 | 0.0665 | 0.8395 | 1.5205 | 1.2315 | 1.7935 | 0.9075 |
| HIST1H2B | 0.66 | 0.678 | 0.053 | 1.188 | 1.686 | 1.434 | 1.506 | 1.023 |
| HIST1H2B | −0.1545 | 0.2405 | 0.0725 | 0.5235 | 1.2935 | 0.5855 | −0.2665 | 0.0845 |
| HIST1H2B | 0.7895 | 0.8975 | −0.1735 | 0.2905 | −0.5625 | −0.0105 | 0.8985 | −0.1325 |
| HIST1H3C | 0.8045 | 0.6195 | 0.2725 | −0.1745 | 0.4485 | −0.0135 | −0.7245 | 1.1415 |
| HM13 | 0.4105 | 1.1945 | 1.6075 | 0.4635 | 0.0745 | 0.3295 | 0.8385 | −0.2205 |
| HMGB3 | −0.1745 | −0.0035 | −0.3405 | −0.0795 | 0.5595 | −0.2095 | −0.1155 | 0.0105 |
| HN1 | 0.055 | 0.093 | −0.208 | 0.02 | −0.105 | 0.602 | −0.134 | −0.454 |
| HNRPA3P | −0.2115 | −0.0685 | −0.6035 | 0.2035 | −0.8705 | −0.2165 | −0.2935 | 0.1815 |
| HNRPDL | −0.088 | 1.878 | −1.525 | −0.514 | −0.676 | −0.302 | −0.871 | 0.536 |
| HOXA5 | 1.6815 | −0.6935 | 1.8635 | 1.7105 | −1.8265 | 1.1965 | 0.6205 | −1.6585 |
| HOXA7 | 0.9475 | 0.8665 | 2.8145 | 3.4085 | −1.2575 | −0.8725 | 0.0985 | −0.0215 |
| HOXB2 | −0.1075 | 1.9855 | 3.2905 | 1.1185 | −1.2075 | −1.4075 | −1.4045 | 1.2655 |
| HOXB6 | 0.5145 | 2.7055 | −0.5195 | 1.7175 | −1.1475 | −1.0355 | −0.7095 | 1.1675 |
| HOXB7 | 1.2045 | 1.6125 | 0.4595 | 0.4345 | 0.1485 | −0.9415 | −0.2755 | 0.2215 |
| HOXC10 | 0.109 | 1.454 | −0.252 | 0.257 | 0.718 | −0.123 | 0.467 | −0.036 |
| HOXC13 | −0.9735 | −0.8805 | 0.2565 | −0.4865 | −1.2995 | −1.3895 | −0.5395 | 0.3205 |
| HOXC9 | 0.023 | −0.463 | −0.636 | −0.701 | −0.195 | 0.423 | 0.432 | −0.264 |
| HOXD3 | 0.2475 | 0.5985 | 0.1785 | 0.2575 | 0.7185 | 0.2625 | −0.4315 | 0.8885 |
| HPCL2 | −0.451 | −1.015 | 0.293 | −0.346 | −2.276 | −1.433 | −0.78 | −0.879 |
| HPS3 | −0.0005 | −0.2875 | 0.6565 | 0.0405 | −0.1815 | 0.2105 | −0.1775 | 0.0465 |
| HRASLS | −0.8375 | 0.1845 | −0.9035 | −0.0845 | −0.4645 | −0.8485 | −0.6915 | −0.2565 |
| HRB | −1.896 | −0.42 | −0.226 | −1.341 | −2.359 | −0.535 | −0.218 | 0.234 |
| HRSP12 | −0.359 | 0.732 | −0.091 | 0.132 | −0.734 | 0.19 | −0.023 | 0.683 |
| HSD17B2 | −0.4425 | 1.0515 | 0.1845 | 0.2075 | −0.0005 | 0.1225 | −0.3615 | 0.7375 |
| HSF1 | −0.5465 | 1.8945 | 0.8695 | 0.5025 | −0.2535 | −0.2785 | −0.7175 | 0.5905 |
| HSPA14 | 0.158 | 0.988 | 1.878 | −0.511 | −0.999 | 0.689 | 0.845 | 1.016 |
| HSPA2 | −0.1535 | −0.5135 | −0.0885 | 0.1455 | 0.3745 | 0.1005 | 0.1545 | −0.0425 |
| HSPA5BP [symbol] | 0.587 | −0.517 | −0.375 | −0.888 | 0.013 | −0.021 | −0.962 | 0.078 |
| HSPC016 | 0.2 | −0.658 | −0.375 | 0.298 | −0.113 | −0.718 | −0.596 | 0.749 |
| HSPC138 | 0.55 | 0.908 | 0.828 | 0.438 | 1.309 | 0.496 | 0.388 | 1.174 |
| HSPC148 | 1.1255 | 0.8685 | −0.4705 | 0.4115 | 0.5905 | 0.6075 | −0.1175 | −0.1205 |
| HSPC150 | −0.6655 | 0.7655 | −0.0815 | −1.1116 | 0.1435 | 0.2545 | 0.4175 | 0.2185 |
| HSPC163 | 1.056 | 0.196 | −0.884 | −0.907 | −0.798 | −0.313 | 0.518 | −0.067 |
| HSPC266 | 0.706 | −0.675 | 0.675 | 1.148 | 0.849 | 0.737 | 0.643 | 0.2 |
| HSU79274 | −0.192 | −0.23 | −0.653 | −0.026 | −0.091 | 0.31 | 0.195 | −0.24 |
| HTATIP2 | 0.424 | 1.359 | 0.221 | 0.58 | 0.15 | 0.237 | 1.017 | 0.304 |
| ICMT | −1.336 | −1.131 | −1.805 | −1.38 | 0.527 | −0.485 | −1.732 | 0.583 |
| ICT1 | −1.168 | 0.105 | 0.554 | 1.154 | 0.099 | −0.414 | 0.696 | −1.257 |
| ID4 | 0.5505 | −0.1735 | −0.4415 | 0.0895 | 0.3555 | 0.4105 | 1.4675 | −0.4965 |
| IDH2 | 1.718 | 0.251 | 0.474 | 0.82 | 0.626 | 0.44 | 0.115 | −0.258 |
| IDS | −0.0365 | 0.6316 | −0.2745 | 0.7375 | 1.3725 | 0.0055 | −0.5375 | −0.5345 |
| IFI30 | −0.1035 | 0.6205 | −0.2705 | 0.4095 | 1.0845 | 0.7555 | −0.5045 | 0.4875 |
| IFI44 | −0.3625 | −0.6585 | −0.9715 | −0.2755 | 1.4295 | 0.1295 | −0.6335 | −0.1875 |
| IFIT5 | 0.2305 | 0.3355 | 0.3975 | 0.8145 | 0.6225 | 0.6065 | −0.3115 | 0.4025 |
| IFITM1 | 0.4775 | 1.0295 | −0.3035 | 1.0535 | 0.1055 | 0.0435 | −0.0445 | −0.2245 |
| IGBP1 | 0.2695 | 0.6965 | −0.1465 | −0.1495 | 1.3335 | 1.7885 | 2.4195 | −0.3305 |
| IGFALS | −0.4515 | 0.2375 | −0.0265 | 0.7675 | 2.9665 | 0.3475 | 2.2795 | 0.9095 |
| IGPBP2 | −0.3765 | −0.1335 | 0.0965 | 1.0415 | 3.2035 | 0.8985 | 1.8425 | 0.8895 |
| IGHG1 | −2.2075 | −0.8795 | −1.1195 | −0.6705 | 1.2565 | 0.6585 | 0.7935 | 2.5685 |
| IGHG1 | −1.091 | 0.06 | 0.282 | 1.831 | 2.66 | 1.208 | 3.084 | 1.065 |
| IGJ | −1.446 | 0.346 | −0.017 | 0.962 | 2.289 | 0.374 | 1.633 | 0.365 |
| IGKC | −0.333 | 0.405 | 0.305 | 0.791 | 2.593 | 0.232 | 1.236 | 0.448 |
| IGKC | −0.336 | 0.2 | −0.342 | 1.328 | 1.948 | 0.171 | 2.064 | 0.806 |
| IGLC2 | 0.1135 | 0.1275 | 0.7695 | 0.2935 | −0.6315 | −0.3735 | −0.7265 | 0.0745 |
| IGLL1 | −0.2395 | −0.3475 | −0.3175 | 0.0025 | −0.6995 | 0.1015 | −0.3235 | 0.5295 |
| IL13RA1 | −0.088 | 0.233 | 0.066 | −0.29 | 0.434 | 0.506 | 0.573 | −0.024 |
| IL17B | 1.2625 | −1.3755 | −1.3215 | −0.2345 | 1.5155 | −0.1635 | −0.8215 | 0.1535 |
| IL23A | 0.1005 | −0.0345 | 0.1755 | −0.1835 | 0.4195 | 0.0075 | −0.0555 | −0.1525 |
| IL6ST | 0.1985 | −0.3215 | −0.9055 | −1.2235 | −0.0065 | −0.7255 | −0.3775 | 0.4475 |
| ILF2 | −0.188 | −0.092 | 0.168 | 0.205 | 1.291 | 1.236 | −0.316 | 1.531 |
| IMPA2 | −1.8325 | 0.0455 | −0.3425 | −0.8175 | 0.2155 | −2.0585 | −0.2445 | 0.5105 |
| ING1L | −0.55 | 0.38 | −0.567 | −0.156 | 0.99 | −0.465 | −0.27 | 0.929 |
| INHBB | 0.1665 | −0.2695 | 0.1225 | 0.0515 | −0.0535 | −0.1535 | −0.4375 | 0.1155 |
| INPP4B | −0.6655 | −1.7615 | −1.3585 | −1.0885 | −1.9885 | −0.9375 | −2.1165 | −0.5785 |
| INSM1 | −0.206 | −0.21 | 0.306 | −0.247 | −1.258 | 0.541 | 1.076 | −0.288 |
| IQCA | 0.63 | −0.492 | −0.389 | 0.324 | 1.142 | 0.106 | −0.487 | 0.078 |
| IQCE | −0.588 | −0.295 | 0.222 | 0.061 | 0.848 | −0.055 | 1.348 | 0.5 |
| IQGAP2 | −0.8845 | 0.8215 | −0.4155 | −0.3875 | 2.0265 | 0.2115 | 2.0975 | 0.8585 |
| IRF4 | −2.3855 | 4.2245 | 1.7515 | −2.0075 | −1.9805 | −1.2685 | −2.9915 | 2.2425 |
| IRTA2 | −0.841 | 0.925 | −0.399 | 0.436 | 1.374 | −0.204 | 0.177 | −0.081 |
| IRX4 | 0.0315 | 0.9575 | −0.3905 | 0.6515 | 1.6015 | 0.2505 | 0.2445 | 0.0735 |
| ISG20 | −1.654 | 1.018 | 0.561 | 0.451 | −0.343 | −0.271 | −2.114 | 0.763 |
| ISGF3 [symbol] | 1.131 | 0.532 | 0.318 | 0.488 | 0.44 | 0.522 | 0.863 | −0.594 |
| ITGB4 | 0.422 | 0.138 | −0.541 | −0.279 | 0.031 | −0.749 | 0.108 | −0.429 |
| ITPR1 | 1.447 | 0.93 | −1.062 | −1.038 | 0.245 | 0.007 | −1.63 | 1.338 |
| JARID1A | −0.479 | 0.318 | −0.802 | −0.486 | −0.171 | 0.121 | −0.482 | 0.611 |
| KARCA1 | −0.271 | 0.307 | −0.125 | −0.511 | −0.495 | 0.288 | 0.09 | −0.373 |
| KATNA1 | 0.5785 | 0.6325 | 0.9295 | 0.6725 | −0.2705 | 0.4705 | −0.2015 | 0.3975 |
| KCNC2 | −0.102 | 0.76 | 0.042 | 0.849 | 0.937 | 0.813 | 0.554 | 0.332 |
| KCND2 | −0.406 | −0.926 | −0.317 | 0.753 | −0.412 | −0.914 | −0.875 | 0.048 |
| KCNK4 | 2.448 | −1.053 | −0.346 | 0.888 | −1.99 | −0.43 | −1.028 | −0.318 |
| KCNN4 | −1.3405 | −0.4545 | −1.2855 | −0.4825 | −0.3665 | −0.5485 | −1.5875 | −0.3505 |
| KCNS3 | 0.2735 | 0.0725 | −0.7125 | 0.1045 | 0.6355 | 1.2235 | −0.1635 | 0.3235 |
| KCTD15 | 0 | −0.438 | −0.171 | 1.085 | 0.16 | 0.871 | −1.373 | −0.305 |
| KCTD3 | −0.665 | −0.557 | −0.538 | −0.748 | −0.362 | −0.273 | −1.051 | −0.335 |
| KDELR2 | −0.263 | 0.115 | 1.218 | 0.222 | 0.759 | 0.283 | 1.357 | 0.924 |
| KIAA0020 | −0.022 | 0.216 | −0.088 | 2.076 | 0.441 | 0.235 | 0.999 | −1.143 |
| KIAA0101 | 0.7585 | 0.9695 | 1.0545 | 0.6245 | 0.0525 | 0.8535 | 0.5775 | 0.8535 |
| KIAA0153 | 0.0016 | 0.2035 | 0.0945 | −0.2125 | −0.0446 | −0.2685 | 1.8845 | 0.0605 |
| KIAA0182 | 0.8615 | 0.3755 | 1.0075 | 0.4485 | 0.0705 | 0.3655 | −0.1475 | 0.1445 |
| KIAA0196 | −0.0275 | −0.1065 | −0.2295 | 0.6945 | 0.4395 | 0.5335 | −0.3305 | −0.3365 |
| KIAA0241 | −0.48 | −1.152 | 2.39 | −0.643 | 0.495 | −0.865 | 0.798 | −0.458 |
| KIAA0251 | 0.3035 | −0.1625 | 0.0645 | −0.2015 | −0.7875 | 0.3905 | 0.1525 | 0.1305 |
| KIAA0274 | −0.296 | 0.118 | −0.557 | −0.589 | 0.813 | 0.308 | 1.224 | 0.774 |
| KIAA0515 | −0.2495 | −0.0805 | −0.4645 | −0.1075 | 1.0935 | −0.1625 | −0.0785 | 0.5195 |
| KIAA0582 | −1.2785 | 3.1765 | 1.1335 | 2.9235 | 0.8655 | 0.7515 | 1.6945 | 0.0355 |
| KIAA0650 | 1.0865 | −1.1885 | −1.5205 | −0.9105 | −0.3965 | −0.5745 | −1.9035 | 1.0105 |
| KIAA0703 | 0.42 | 0.896 | 0.296 | −0.869 | −0.454 | 0.886 | 1.533 | 0.304 |
| KIAA0830 | −1.3395 | 1.0145 | 0.3095 | 0.3655 | 0.4345 | 1.3175 | 0.1845 | 0.3825 |
| KIAA0980 | 0.5315 | 0.5165 | −0.2495 | 0.8955 | 1.1575 | −0.1635 | −0.0495 | −0.3815 |
| KIAA1324 | −0.462 | 1.642 | −0.305 | −0.31 | −0.104 | −0.168 | 0.938 | 0.829 |
| KIAA1363 | 0.9 | 0.897 | 0.014 | −1.127 | −0.682 | −0.015 | −1.989 | −0.872 |
| KIAA1683 | −0.8135 | −0.3855 | 0.2035 | −0.8935 | 1.1795 | 0.4865 | 0.0015 | 0.5255 |
| KIF12B | −0.5795 | 0.1445 | −0.4815 | −0.3885 | 0.1095 | −0.5125 | −0.5355 | 0.3845 |
| KIF24 | −0.8215 | 0.0365 | −0.0375 | −0.0025 | −0.0285 | 0.4515 | 0.2305 | 0.0155 |
| KIN | −1.0785 | −1.8285 | −1.6485 | −1.3215 | 1.3465 | −0.4575 | −0.9615 | 1.4475 |
| KIRREL3 | −0.4235 | −0.2176 | −0.4645 | −0.3305 | −0.1565 | −0.0735 | −0.4755 | 0.0025 |
| KIT | −1.7725 | −0.3885 | −1.2815 | −0.8435 | −1.5425 | −0.0895 | −1.9195 | 0.1745 |
| KLK5 | −0.981 | −0.971 | −0.395 | 0.483 | −0.346 | −0.4 | −0.844 | 0.069 |
| KLK6 | −0.891 | −0.828 | −0.276 | −0.582 | −1.379 | −0.491 | −0.809 | 0.055 |
| KLK7 | −0.288 | 0.596 | 0.6 | 0.165 | 0.796 | −0.269 | 0.853 | 1.06 |
| KLK8 | 0.2585 | 0.8465 | 0.5505 | 1.0895 | 1.7995 | 1.8655 | 1.8305 | −0.1265 |
| KNTC2 | −0.238 | −0.348 | 0.472 | 2.902 | −0.155 | 0.831 | 0.744 | −0.322 |
| KPNA2 | 0.4865 | −0.5155 | −0.8445 | −0.7935 | 2.4705 | 0.4055 | −1.1045 | 0.7945 |
| KREMEN2 | −1.0425 | −1.1465 | −0.8935 | −1.5475 | −0.6855 | −1.7995 | −1.3765 | 0.3445 |
| KRT10 | −1.2085 | −1.3945 | −1.1715 | −1.8815 | −0.7445 | −2.2765 | −2.2135 | 0.8075 |
| KRT13 | −1.081 | −1.111 | −0.909 | −0.323 | −1.189 | −0.321 | −0.901 | −0.402 |
| KRT15 | 1.09 | 1.052 | 1.672 | 0.428 | 0.348 | 0.229 | −0.285 | 1.317 |
| KRT17 | −0.6885 | −2.9615 | −2.6485 | −0.8785 | −2.9605 | −2.3295 | −2.5305 | −2.4175 |
| KRT18 | −2.317 | −2.848 | −1.283 | −0.101 | −2.471 | 0.48 | −3.238 | −1.761 |
| KRT23 | 0.9095 | 0.0215 | −0.1375 | −0.1975 | 0.0635 | −0.0165 | −0.0365 | 0.0425 |
| KRT6B | 0.318 | 0.644 | 0.06 | −0.149 | 1.334 | 0.171 | 0.195 | 0.833 |
| KRTAP3-3 | 0.7365 | −1.1215 | −0.3925 | −1.1585 | 2.2255 | 1.1175 | 1.5535 | 1.1185 |
| KRTHA7 | −1.8055 | −1.1325 | −1.5985 | −1.0755 | −2.2755 | −0.9955 | −2.0955 | −0.0245 |
| LAMA3 | 0.766 | 0.503 | 0.385 | 0.676 | 0.277 | 0.584 | 0.096 | 0.257 |
| LAMB3 | −9.21 | 0.248 | −0.139 | 0.79 | 1.969 | 0.336 | 0.478 | 0.382 |
| LASS2 | −0.4435 | −0.2975 | −0.9245 | −0.3035 | 0.4645 | 0.5555 | −0.2865 | −0.1645 |
| LAX | −0.8255 | −1.1325 | 0.7215 | −0.9945 | −1.9835 | −1.5655 | −0.8845 | 0.6895 |
| LBR | 0.5145 | −1.1245 | −0.7135 | −0.1475 | −0.7325 | −0.8215 | 0.8985 | −1.0695 |
| LCE1B | 0.0075 | 0.2385 | 0.3685 | −0.9015 | −0.4655 | 0.5825 | −0.2285 | −0.8865 |
| LDHB | 0.603 | −0.62 | 0.441 | −1.292 | −0.062 | 0.016 | −0.136 | 0.064 |
| LDOC1L | 0.065 | 2.37 | 0.535 | 0.815 | −0.105 | 0.99 | 0.707 | 1.881 |
| LETMD1 | −2.3965 | −1.7435 | −0.7665 | −0.6905 | 2.1945 | 1.9065 | 0.6815 | 1.7816 |
| LFNG | −0.008 | 0.345 | −0.438 | −0.015 | 0.148 | −0.048 | −0.133 | −0.026 |
| LGALS2 | 0.257 | 0.654 | 1.241 | 1.097 | 0.495 | 0.137 | 0.579 | 0.471 |
| LGTN | 0.762 | 0.203 | −0.479 | −1.327 | 1.204 | 0.495 | −1.317 | 0.212 |
| LNX | −0.2125 | 0.8025 | 0.0835 | 0.6075 | 0.0535 | 0.3035 | 0.2075 | 0.3155 |
| LOC11247 | −0.701 | 1.046 | 0.528 | −1.023 | 0.615 | −0.067 | −0.843 | 0.568 |
| LOC11492 | −1.19 | 0.103 | 0.088 | 0.085 | 0.503 | −0.254 | 0.336 | 0.047 |
| LOC11497 | −0.3255 | −0.2615 | −0.0985 | −0.1935 | 0.1725 | −0.1285 | −0.2025 | −0.1615 |
| LOC11621 | 0.4035 | 0.6245 | 2.8675 | −0.2385 | 0.6255 | −0.0775 | 1.0225 | 0.5325 |
| LOC11644 | 0.5835 | 1.2735 | −1.2805 | 0.0425 | 0.7205 | −0.7735 | −0.4955 | 0.3565 |
| LOC12022 | 0.0215 | 0.4255 | 0.0285 | 0.4095 | 0.0025 | 0.3815 | 0.1325 | −0.0716 |
| LOC12422 | 0.6165 | −0.6045 | 0.4035 | −2.3105 | −1.5075 | −0.7565 | 0.7555 | −1.0935 |
| LOC14654 | −0.74 | 1.177 | 0.52 | 0.584 | 0.782 | 0.863 | 0.442 | −0.005 |
| LOC15506 | −0.142 | −0.317 | 0.882 | −1.161 | −1.055 | 0.006 | 0.294 | 0.556 |
| LOC20189 | −0.9346 | 0.1405 | −0.6415 | −0.2505 | −1.4695 | −0.7525 | −0.7595 | 0.1205 |
| LOC25398 | −0.283 | 0.749 | 0.514 | 0.56 | −0.156 | 0.088 | 0.6 | −0.055 |
| LOC33990 | −0.869 | 0.858 | 0.416 | 0.264 | 1.069 | 0.261 | 1.081 | −0.645 |
| LOC34817 | 0.8075 | −0.6235 | −0.0395 | −1.0275 | −0.4885 | −0.1825 | 0.0205 | 0.3665 |
| LOC40045 | 0.182 | −1.54 | 0 | 0.371 | −0.826 | −0.917 | −1.206 | −0.66 |
| LOC44089 | −0.111 | 0.116 | −0.386 | −0.493 | −0.611 | −0.362 | 1.865 | 0.051 |
| LOC49386 | −0.98 | −1.067 | −0.866 | −0.588 | −0.122 | −0.535 | 0.186 | 0.038 |
| LOC51123 | 0.819 | 0.437 | 0.148 | 1.35 | 2.225 | 0.503 | 1.092 | 0.143 |
| LOC51161 | 1.6805 | 1.2895 | 0.6305 | −0.6625 | −0.3275 | −1.0885 | −1.1885 | −1.3316 |
| LOC51321 | −0.2415 | 1.8995 | 1.8245 | −0.3045 | −0.3105 | 2.8585 | 0.5575 | 0.4095 |
| LOC51760 | −0.2945 | 1.9075 | 0.3855 | 0.0345 | −0.6695 | −0.2715 | 0.7525 | −0.7755 |
| LOC81558 | 0.395 | 0.45 | 0.333 | −0.274 | 0.316 | 1.289 | 0.203 | 0.472 |
| LOC81569 | 0.2835 | 0.0035 | −1.0595 | 0.8545 | 0.3415 | −0.1265 | 0.7285 | 0.4225 |
| LOC90355 | 0.067 | −0.06 | 0.226 | 0.758 | 0.652 | −0.582 | 0.547 | 0.087 |
| LOC90701 | 1.995 | −0.406 | 0.077 | 1.205 | −0.739 | 1.106 | −0.201 | 1.454 |
| LONP | 0.3965 | 0.2835 | 0.5955 | 0.1105 | 0.4235 | 0.5415 | 0.9795 | 1.1345 |
| LRAP | −0.6945 | −1.0185 | −0.5345 | 0.1945 | 0.2955 | 1.1735 | 0.2545 | −1.0285 |
| LRBA | 1.3415 | 0.3885 | −0.5905 | −0.2285 | −0.2525 | −0.9545 | −0.6385 | −0.1405 |
| LRIG1 | −0.1635 | 1.0605 | 0.5465 | −0.2835 | −0.8575 | −0.5125 | 0.3045 | 0.9095 |
| LRP6 | −1.108 | −0.307 | −0.002 | 0.978 | −0.313 | −0.339 | −1.036 | −0.044 |
| LRP8 | −0.138 | −0.311 | 0.373 | 0.923 | 1.242 | 0.558 | 1.035 | 0.105 |
| LRRC17 | −0.1645 | −0.0365 | −0.2505 | −0.7035 | −0.1735 | −0.2025 | 0.4685 | 0.3205 |
| LRRC2 | −0.1555 | −0.0415 | 0.0155 | −0.8255 | 0.4475 | −0.2535 | −0.1935 | −0.0195 |
| LSM1 | 0.1415 | 0.3875 | −0.2675 | 1.2395 | 1.8735 | 1.1185 | −0.0275 | 1.1905 |
| LSM8 | −0.169 | 2.516 | −0.228 | 0.181 | −0.105 | 0.009 | −0.15 | 0.652 |
| LTB | 0.981 | −0.378 | −0.7 | 0.758 | 0.9 | −0.272 | −0.375 | 0.176 |
| LY6D | −0.085 | 0.6 | 0.968 | 0.665 | 1.455 | 0.805 | 0.495 | 1.186 |
| LYN | 0.657 | −0.314 | −0.004 | −0.45 | 0.103 | −0.253 | −0.062 | 0.189 |
| MA02L1 | −0.155 | 0.274 | −0.078 | 0.85 | 1.413 | −0.105 | 0.468 | 0.398 |
| MADP-1 | −0.0125 | −0.2765 | 0.1285 | 0.5795 | 2.7075 | 0.3085 | 0.5535 | −0.3125 |
| MAGEA12 | −0.077 | 0.27 | 0 | 0.645 | 1.868 | −0.428 | −0.338 | 0.34 |
| MAGEA2 | −0.6825 | −0.2155 | 0.4375 | 0.6035 | 2.5435 | −0.6735 | 0.9095 | −0.3465 |
| MAGEA3 | −0.5825 | −0.8295 | 0.7345 | −0.9035 | −2.3095 | −1.5045 | 0.5945 | 0.6445 |
| MAGEA4 | −2.171 | −0.167 | −0.336 | −2.089 | −1.35 | −1.162 | −1.824 | −1.401 |
| MAGED4 | −0.5485 | −0.2355 | 0.8175 | 0.8975 | 0.5755 | −0.0895 | −0.0025 | 0.7905 |
| MAGI1 | −0.6255 | 1.0945 | 0.0035 | 0.0405 | −0.6545 | −0.7205 | 0.5035 | 0.0465 |
| MAGI-3 | 0.467 | 0.319 | −0.119 | 0.254 | −0.128 | 0.138 | −0.031 | −0.119 |
| MAL2 | −0.022 | −1.066 | −0.877 | 0.761 | −0.238 | 1.049 | 1.114 | 0.896 |
| MAN2B1 | 0.9705 | 1.1325 | −0.7075 | −0.2065 | 0.8825 | 0.3845 | −0.8745 | −0.4485 |
| MAPK8IP2 | −0.7475 | 0.9045 | 0.7945 | 1.1855 | 1.2565 | 0.2725 | 0.7515 | 0.4425 |
| MARCO | 1.555 | 1.598 | 0.127 | 2.149 | 0.913 | 0.653 | 0.163 | 0.247 |
| MARLIN1 | −0.102 | 0.222 | 0.373 | 0.328 | −0.141 | 0.025 | 0.195 | 0.183 |
| MASS1 | 0.2115 | 0.3965 | 0.0716 | −0.7855 | 0.1865 | −0.0485 | 0.1225 | 0.3685 |
| MCM2 | −1.8095 | −0.6295 | −1.1965 | −0.5715 | −0.1355 | −0.7055 | −0.7355 | −1.1415 |
| MCM3 | 0.41 | 1.103 | −1.911 | 0.753 | −0.011 | 0.253 | 0.758 | 0.404 |
| MESP1 | 0.2745 | −0.6335 | 0.1115 | 0.3785 | −0.2025 | −0.0265 | −2.3405 | −0.7055 |
| METRN | 0.1015 | −0.7016 | −0.6385 | 0.1715 | −0.2175 | −0.0135 | 0.0885 | −0.2245 |
| MFAP2 | 0.408 | 0.456 | −0.052 | −0.14 | −0.767 | 0.227 | −0.216 | 0.012 |
| MFGE8 | 0.0815 | −0.0685 | −0.9865 | 0.5065 | −1.0185 | −0.0445 | −0.3755 | −0.1495 |
| MGAT4B | −0.163 | 0.434 | −0.078 | 0.693 | 0.669 | 0.288 | 0.025 | 0.088 |
| MGC1091 [symbol] | 1.2675 | 1.5895 | −0.1845 | 0.2815 | −1.0275 | 1.3015 | 0.4395 | 1.4945 |
| MGC1098 [symbol] | 0.8916 | −0.4725 | 0.3125 | −0.7835 | 0.4015 | −0.7125 | −0.0065 | 0.0855 |
| MGC1124 [symbol] | −0.377 | −0.428 | −0.454 | −0.789 | −0.422 | −0.13 | −0.208 | 0.084 |
| MGC1127 [symbol] | 0.34 | −0.513 | 0.31 | −0.547 | 0.274 | −0.1 | 0.398 | −0.51 |
| MGC1312 [symbol] | 0.709 | −0.493 | 0.561 | 0.228 | 0.589 | 0.644 | −0.378 | 0.123 |
| MGC1318 [symbol] | 0.688 | 0.041 | −0.137 | −0.543 | 0.067 | 0.084 | 0.125 | −0.174 |
| MGC1414 [symbol] | 0.4595 | 0.5795 | −0.0295 | 0.7935 | 0.2175 | −0.1505 | −0.0955 | −0.2625 |
| MGG1428 [symbol] | 1.558 | 0.384 | −0.65 | −0.88 | 1.385 | 0.491 | −0.333 | −0.34 |
| MGC1481 [symbol] | −0.328 | 1.7 | 0.27 | 1.44 | 1.284 | 1.384 | 0.116 | −0.318 |
| MGC1580 [symbol] | 0.4155 | −0.2345 | 0.0965 | 0.0195 | −0.2095 | −0.1565 | −0.5705 | 2.3445 |
| MGC1663 [symbol] | 0.273 | 0.875 | 0.315 | 0.12 | 0.831 | −0.008 | −0.38 | 0.492 |
| MGC1873 [symbol] | 0.887 | −0.345 | −0.111 | −0.212 | −0.605 | −0.517 | −0.257 | −0.225 |
| MGC2053 [symbol] | 0.264 | −0.673 | 0.372 | −1.268 | 0.54 | 0.517 | 0.13 | −0.741 |
| MGC2398 [symbol] | −0.185 | 0.695 | −0.243 | −0.484 | −0.357 | −0.629 | −0.821 | −0.487 |
| MGC2610 | 0.131 | 0.38 | −0.004 | 1.691 | 3.105 | 0.213 | 1.106 | −0.255 |
| MGC2714 | −0.595 | 0.67 | 0.004 | 1.418 | 3.171 | 0.245 | 1.279 | 0.639 |
| MGC2716 [symbol] | −0.6175 | 0.7395 | −0.0785 | 1.5885 | 2.8355 | 0.0135 | 2.4135 | 0.6005 |
| MGC2716 [symbol] | −0.7955 | 0.6045 | 0.3986 | 2.2975 | 2.4615 | 0.4475 | 1.8885 | 0.8295 |
| MGC2716 [symbol] | −0.798 | 0.551 | −0.08 | 1.277 | 2.541 | 1.029 | 1.598 | 1.014 |
| MGC2716 [symbol] | 0.6695 | −0.2525 | −0.6585 | −0.5775 | −0.3535 | 0.1745 | 0.0025 | 0.1365 |
| MGC2716 [symbol] | 0.099 | 0.1 | −0.187 | 0.188 | −0.492 | 0.424 | −0.748 | −0.595 |
| MGC2887 [symbol] | −0.845 | −0.399 | −0.408 | −1.506 | −0.928 | −0.523 | 0.65 | 0.478 |
| MGC3212 [symbol] | −0.425 | −0.238 | −1.511 | −0.441 | 0.09 | −0.326 | −0.878 | 0.482 |
| MGC3321 [symbol] | −0.8325 | 0.5476 | −0.2975 | −0.7165 | 0.8135 | 0.0926 | −0.4375 | −0.6885 |
| MGC3484 [symbol] | −0.7365 | 0.7176 | 0.4585 | −0.5725 | 0.7645 | 0.1845 | 0.2335 | 0.0335 |
| MGC3492 [symbol] | 0.681 | 0.053 | −1.493 | −1.193 | −0.18 | −0.429 | −0.454 | 0.02 |
| MGC4021 [symbol] | 0.131 | −1.087 | 0.023 | 0.301 | −0.318 | −0.819 | −0.647 | −0.632 |
| MGC4251 | 0.5895 | 0.4376 | −0.2055 | −0.8025 | 0.2745 | 0.4015 | −1.4865 | 0.3655 |
| MGC4308 | 1.146 | 1.791 | −1.109 | 0.258 | −0.178 | −0.175 | 1.368 | 1.367 |
| MGC4800 | −2.0725 | −1.8755 | −1.6855 | −1.1135 | −1.1925 | −0.0345 | −1.7875 | 0.4045 |
| MGC4659 | −1.348 | −1.203 | −0.737 | −0.883 | −0.635 | −0.455 | −1.65 | −0.231 |
| MIA | −2.0265 | −1.8005 | −0.5085 | −0.7685 | −1.1475 | −0.9305 | −1.8855 | −0.1605 |
| MID1 | −2.5765 | −2.4045 | −1.2535 | −2.2105 | −1.4545 | −0.6315 | −1.6615 | −0.1545 |
| MID1 | 0.112 | −0.442 | 1.922 | 0.089 | 1.342 | −0.135 | 0.774 | 1.584 |
| MID1 | −0.3205 | 0.6715 | −0.8115 | −0.1485 | 0.1225 | 0.9645 | 0.1495 | −0.3475 |
| MLF1IP | 2.2395 | −1.0806 | 3.8145 | 1.2175 | −1.9085 | −1.4835 | −1.3465 | −1.4965 |
| MLSTD1 | 0.783 | −1.14 | 0.192 | −0.228 | −1.37 | −2.05 | −2.722 | −0.96 |
| MMP1 | 0.479 | 1.13 | −0.761 | 0.381 | 1.221 | 0.776 | 0.917 | 0.406 |
| MMP7 | 0.4795 | −0.5765 | −0.3985 | −0.4385 | 0.4365 | 0.5185 | −0.2325 | 0.2565 |
| MNAT1 | −0.4715 | −0.0975 | −0.0435 | 0.0085 | −0.4545 | 0.1345 | 0.0855 | −0.5745 |
| MRPL14 | 0.453 | 1.29 | 2.7 | 0.868 | −0.802 | 1.405 | 0.131 | 0.348 |
| MRPL15 | 0.0165 | 0.0675 | −0.4145 | −0.0945 | 0.4355 | −0.0795 | −0.0175 | 0.1895 |
| MRPL27 | 0.3535 | 0.3655 | 0.0895 | −0.5005 | 0.6435 | 0.8665 | 0.2025 | −0.2525 |
| MRPL34 | 0.977 | −0.607 | 2.359 | −0.149 | 2.085 | 0.263 | −0.982 | 0.597 |
| MRPL43 | −0.0795 | −0.0785 | −0.1885 | 0.0825 | −0.5535 | −0.3045 | −0.2885 | −0.4955 |
| MRPL45 | 0.305 | 0.183 | 0.008 | −0.131 | 0.037 | 0.083 | −0.209 | 0.066 |
| MRPL48 | 0.9075 | 0.3265 | −0.4105 | −0.4395 | −0.2365 | −0.2435 | −0.9316 | 0.0935 |
| MRPL9 | −0.3105 | 0.4055 | 0.3215 | 0.4875 | −0.0795 | −0.0405 | 0.4785 | 0.4745 |
| MRPS14 | 0.408 | 0.365 | −1.332 | 0.921 | 0.098 | −0.147 | −0.537 | 0.194 |
| MRPS15 | 0.381 | 0.572 | −0.443 | 0.139 | −0.734 | 0.207 | −0.464 | 0.495 |
| MRPS17 | −0.067 | 1.412 | 0.125 | −0.414 | −0.19 | −0.001 | −0.347 | −0.407 |
| MRPS24 | −0.7265 | 0.4075 | −0.4885 | −0.3565 | −0.1285 | −0.0335 | −0.1145 | −0.1495 |
| MRPS25 | −0.552 | 0.079 | −0.328 | −0.371 | −0.357 | −0.541 | −0.578 | −0.434 |
| MRPS31 | −0.395 | −0.037 | −0.108 | 0.408 | 1.923 | 0.539 | 0.284 | 0.887 |
| MRPS6 | −0.5395 | 0.3105 | 0.1555 | −0.6725 | −0.1395 | 0.0505 | −0.3055 | 1.0015 |
| MS4A1 | 0.3105 | 0.0025 | 0.0485 | −0.1325 | −0.0285 | −0.6795 | 0.1735 | 0.6875 |
| MSH2 | −0.841 | 3.429 | −0.209 | 3.999 | 5.34 | 5.055 | 0.62 | 4.395 |
| MSH3 | −0.184 | 0.433 | −0.208 | 0.699 | 0.283 | 0.168 | 0.112 | 0.296 |
| MSMB | −0.152 | 0.02 | 0.746 | −0.099 | −0.234 | 0.455 | 0.05 | 0.525 |
| MTA1 | −0.5215 | 1.3965 | −0.7385 | −0.3535 | 1.9865 | −0.1445 | 0.4615 | 0.1805 |
| MTA3 | −0.314 | −0.091 | 0.262 | −0.407 | −0.533 | 0.404 | 0.747 | −0.41 |
| MTAC2D1 | 0.37 | −0.298 | −0.01 | −0.012 | 0.344 | 0.139 | 0.496 | 0.291 |
| MTCH1 | 0.6955 | −0.3135 | −0.2735 | −0.1965 | −0.1985 | −0.9725 | −0.4265 | 0.4105 |
| MTHFD1 | −3.7325 | −0.8875 | −0.8835 | 0.3015 | −0.6105 | 0.6625 | 0.2085 | 0.7085 |
| MTMR2 | −0.0145 | 9.9185 | 0.0785 | 1.2185 | 0.0145 | −0.6415 | −0.4885 | −0.1655 |
| MUC1 | −0.934 | 0.281 | −0.617 | −0.848 | −0.496 | −0.457 | 0.605 | −0.008 |
| MUC20 | 0.587 | 0.116 | −0.779 | −0.587 | 0.538 | 0.48 | −1.206 | −0.239 |
| MUM1 | 0.402 | 0.277 | −1.227 | −1.21 | −0.387 | 0.843 | 1.179 | 0.707 |
| MXI1 | 0.404 | 0.398 | 0.986 | −0.017 | 2.755 | 1.228 | −0.467 | 1.368 |
| MYB | 1.151 | 0.814 | 1 | 0.138 | 0.515 | 0.638 | 1.041 | 1.713 |
| MYBL1 | −0.581 | 0.002 | 0.846 | −1.137 | −1.847 | −1.149 | −2.226 | −1.157 |
| MYBL2 | −0.687 | 0.667 | −0.088 | −0.034 | 0.313 | 0.073 | 1.249 | 0.196 |
| MYO10 | 0.1105 | 0.1905 | −0.2385 | 0.0235 | 0.1425 | −0.2265 | 0.7915 | 0.1965 |
| MYO6 | −1.9115 | 1.2655 | 1.1885 | −1.6575 | −0.7225 | −1.3285 | −1.0735 | 1.0555 |
| MYST3 | 0.2805 | 0.2625 | −0.1295 | −0.2295 | −0.0145 | −0.1455 | −0.0305 | 0.9495 |
| NALP2 | 0.2495 | −0.0965 | 0.3295 | 1.2945 | 0.0645 | 0.4875 | 0.1815 | 1.2225 |
| NALP6 | 0.3865 | 0.7805 | 0.5795 | −0.5495 | 0.5535 | −0.6915 | −0.4935 | 0.1155 |
| NANS | −0.214 | 0.012 | −0.42 | 2.918 | 2.893 | 1.528 | −0.606 | 0.437 |
| NARF | −0.008 | −0.989 | −0.815 | −1.693 | −1.093 | −1.3 | −1.302 | −0.604 |
| NAT2 | 0.3375 | −0.0255 | 0.0055 | 0.4355 | 0.1835 | −0.3745 | −0.0005 | 0.7175 |
| NAV2 | 0.781 | 0.206 | 2.795 | 1.045 | −0.654 | 1.555 | 0.534 | 0.527 |
| NDE1 | −0.3585 | −1.5905 | −0.9795 | −1.2365 | −1.3745 | −0.9545 | −1.2075 | 0.3335 |
| NDP52 | 0.505 | 0.351 | −0.425 | −0.283 | 0.415 | 0.527 | 1.072 | 0.554 |
| NDRG2 | 0.532 | 0.642 | −0.248 | 0.288 | 1.242 | −0.248 | −0.257 | 1.422 |
| N0UFA7 | 0.4835 | 0.7075 | −0.2605 | 9.4905 | 0.4625 | 0.4785 | −0.0605 | 0.4285 |
| NDUFB1 | −0.235 | −0.095 | −0.193 | −0.447 | 0.078 | 0.223 | 0.186 | 0.3 |
| NDUFB10 | 0.318 | 0.153 | 0.189 | −0.283 | 0.089 | 0.315 | −0.063 | −0.01 |
| NDUFB2 | −0.1495 | −0.3295 | −0.4225 | −0.5995 | 0.2835 | −0.1335 | −0.8615 | 0.6255 |
| NDUFB5 | 0.1895 | −0.2825 | 0.2885 | −1.0585 | −0.8455 | 0.3585 | −0.2055 | 3.2285 |
| NDUFB6 | 1.4575 | 1.7175 | −0.1405 | 0.4235 | 1.5275 | 0.1125 | 0.3405 | 0.6795 |
| NDUFC2 | 9.0825 | 0.1015 | −0.0835 | 0.1045 | 1.0885 | 0.3565 | 0.2705 | 0.8235 |
| NEBL | 0.163 | −0.31 | −0.001 | 0.355 | −0.796 | −0.911 | −0.675 | −0.396 |
| NEK2 | 3.776 | −0.534 | −0.001 | 2.078 | 0.802 | 0.244 | 0.001 | 0.611 |
| NEK6 | 0.3665 | 1.3195 | 0.1115 | −0.6975 | −0.9495 | −0.8335 | −0.2855 | 0.9975 |
| NELL2 | −0.3965 | −1.0355 | −0.2655 | 0.1025 | 0.8285 | 0.3865 | −0.1265 | 0.1245 |
| NEURL | −0.127 | 0.992 | −1.275 | −0.608 | −0.412 | −0.322 | −2.357 | −0.737 |
| NFE2L3 | −1.8845 | −0.8635 | −0.9955 | −0.9465 | −0.3985 | −0.1825 | −2.1025 | −0.2455 |
| NFIA | 0.764 | 0.402 | 1.433 | 0.65 | 0.783 | −0.034 | −1.388 | 0.151 |
| NFIB | 0.0405 | 0.0005 | 0.9885 | −0.7985 | 0.3005 | 0.3445 | 0.0355 | 0.4905 |
| NINJ1 | 0.0175 | 0.3865 | 0.1315 | 0.1725 | 1.3015 | 0.0465 | −0.4615 | 0.1445 |
| NIPSNAP [symbol] | 1.1265 | 0.6735 | 2.6225 | 1.6945 | −1.1965 | 0.7245 | −0.4105 | 0.5835 |
| NKX8-2 | 0.9845 | 0.7035 | 2.4715 | 1.6315 | −1.5335 | 1.1316 | −0.5815 | 0.4445 |
| NME1 | 0.293 | 0.063 | −0.066 | 0.16 | 0.664 | 0.795 | 0.207 | 0.307 |
| NME2 | 0.8815 | 0.4385 | −0.7545 | 0.9985 | 0.9195 | 2.1765 | −0.5255 | 1.9845 |
| NME3 | −1.2785 | −0.9695 | −0.2595 | 0.1205 | 1.2435 | −0.0025 | −1.0595 | −0.8095 |
| NMES1 | −0.249 | 0.209 | 0.332 | 0.5 | 0.024 | −0.215 | 0.477 | −0.208 |
| NMI | −0.2065 | −0.1125 | −0.1035 | −0.5575 | −0.5185 | 0.3405 | 0.5565 | −0.1225 |
| NOC4 | −0.4705 | 0.1585 | −0.0385 | 0.5765 | 3.2345 | 0.2815 | 1.8935 | 0.9315 |
| N0LC1 | −0.4885 | −1.5915 | −1.2315 | −0.9255 | −0.9385 | −0.8035 | −0.8475 | −0.9535 |
| NotFound | −0.8465 | 0.2175 | −0.1295 | −0.4116 | −0.3415 | 0.4955 | 0.4175 | 0.2895 |
| NotFound | −0.689 | 0.263 | −0.119 | 0.745 | 1.331 | 0.102 | 1.178 | 0.492 |
| NotFound | 0.071 | 0.724 | 2.305 | −0.115 | 1.2 | −0.277 | 0.057 | −0.794 |
| NotFound | −0.149 | 0.028 | −0.28 | 1.357 | 2.223 | 0.589 | 0.208 | 0.435 |
| NotFound | −0.0135 | −0.9835 | 0.6885 | 1.1935 | 1.5215 | 0.5885 | 0.7985 | 0.2876 |
| NotFound | −0.935 | −0.934 | −1.134 | 1.01 | 0.201 | −0.182 | −0.672 | 0.037 |
| NotFound | 0.8565 | 1.0555 | 2.2685 | 0.7835 | 1.5955 | 2.0125 | 2.1585 | 0.5545 |
| NotFound | −0.388 | 0.221 | 0.524 | −0.308 | 1.089 | −0.459 | −0.973 | −0.486 |
| NotFound | −0.7405 | −0.4545 | 0.2145 | −0.4435 | −1.3155 | −0.9565 | −0.0405 | −0.5705 |
| NotFound | −0.6475 | 1.0495 | 0.3315 | 1.0545 | 1.9665 | 0.4465 | 1.1325 | 0.6605 |
| NotFound | 0.4245 | 0.4135 | 0.0235 | 0.4025 | 0.1125 | 0.2285 | −0.0185 | 0.5995 |
| NotFound | −0.0705 | 0.3555 | 0.0945 | 0.2875 | 0.0975 | −0.1075 | −0.0195 | −0.3115 |
| NotFound | −0.516 | −0.466 | −1.658 | −0.987 | 2.518 | 2.057 | −2.972 | 0.861 |
| NotFound | −0.217 | 0.775 | 0.111 | −0.049 | 0.081 | 0.145 | 0.043 | 0.151 |
| NotFound | 1.035 | 1.174 | 1.75 | 0.386 | 0.54 | 0.451 | −0.269 | 1.344 |
| NotFound | 1.8235 | 0.7325 | 0.2105 | 0.5155 | 0.1805 | 1.4785 | 1.4075 | 2.1735 |
| NotFound | −0.725 | 0.476 | 1.334 | −0.025 | 0.81 | −0.277 | −0.151 | −0.303 |
| NotFound | −0.359 | −0.84 | −0.697 | −0.818 | −0.428 | −0.685 | −1.013 | −0.343 |
| NotFound | −0.0865 | −0.5995 | −1.1825 | −0.3135 | 0.1695 | 0.4015 | 0.4445 | −0.3045 |
| NotFound | 0.0515 | −1.5565 | −1.6935 | −2.3975 | −0.0745 | 0.4555 | −1.1365 | −0.9675 |
| NotFound | 0.798 | −0.231 | −0.557 | −0.523 | −0.102 | −0.289 | −0.544 | 0.416 |
| NotFound | 0.4585 | −0.2535 | 0.2095 | −0.3255 | −0.8445 | −0.0295 | −0.7935 | −0.1655 |
| NotFound | −0.248 | 0.182 | −0.844 | 3.001 | 2.912 | 2.418 | −1.233 | 1.292 |
| NotFound | 0.539 | 0.608 | 0.282 | 1.199 | 1.755 | 1.107 | 1.449 | 1.176 |
| NotFound | −0.2135 | 0.4385 | −1.0455 | 0.1615 | 0.5885 | −0.4405 | −1.0375 | −0.2215 |
| NotFound | 0.705 | −0.527 | −0.341 | 0.281 | 0.389 | −0.276 | −0.621 | 0.027 |
| NotFound | 0.5175 | 0.4705 | 0.0145 | 1.0435 | 1.5315 | 1.2825 | 1.5475 | 0.8565 |
| NotFound | −0.406 | 2.717 | 0.089 | 0.027 | 4.267 | 2.397 | 2.294 | 0.931 |
| NotFound | 0.529 | −0.487 | −0.429 | −0.123 | 0.532 | 0.735 | 0.422 | 0.308 |
| NotFound | −0.2785 | −0.0045 | 0.0175 | 0.2975 | 1.7305 | 0.5435 | 3.3015 | 0.9945 |
| NotFound | 1.0695 | −0.9655 | 0.2445 | 2.5265 | 1.9205 | 0.7995 | 1.2045 | 0.0065 |
| NotFound | 0.6835 | 1.3235 | 0.3755 | 1.9975 | 1.2245 | 2.2805 | 1.9805 | 0.8095 |
| NotFound | 0.0715 | 0.7805 | 0.1205 | 1.0855 | 1.0925 | 1.8215 | 1.3945 | 0.8905 |
| NotFound | 1.0055 | 0.0805 | −0.4985 | 0.2075 | 0.8725 | 0.4765 | 0.5575 | −0.6295 |
| NotFound | −0.104 | 0.238 | −0.112 | 0.872 | 1.394 | 1.08 | 1.15 | 0.227 |
| NotFound | 0.1195 | −0.0135 | 0.1145 | −0.8365 | 0.6675 | 0.8425 | 1.8265 | 0.4205 |
| NotFound | −0.5955 | 0.2985 | −0.2105 | 0.6775 | −0.1365 | −0.1185 | 1.4885 | 0.0395 |
| NotFound | 0.4685 | 1.1435 | −0.9915 | 1.9105 | 1.6285 | 1.9385 | 1.5185 | −0.1105 |
| NotFound | 1.8935 | 0.4245 | 0.2085 | 2.4885 | 0.3745 | 1.4305 | 0.5375 | 0.3575 |
| NotFound | −0.395 | −0.544 | 0.317 | −1.393 | 0.503 | −0.24 | −0.551 | 0.417 |
| NotFound | 0.088 | 0.222 | 0.316 | 0.29 | 0.514 | −0.478 | −0.082 | 0.214 |
| NotFound | −0.388 | 0.405 | −0.038 | 1.583 | 2.325 | −0.04 | −0.021 | 0.39 |
| NotFound | 0.4645 | 0.7135 | 0.0705 | 1.1965 | 1.8735 | 1.2765 | 1.2785 | 0.9455 |
| NotFound | −0.5425 | −0.3265 | −0.0575 | −0.2025 | 0.9925 | 0.5395 | 0.7825 | −0.4705 |
| NotFound | −0.04 | 0.704 | 0.034 | 0.672 | 0.565 | 0.131 | 0.224 | −0.017 |
| NotFound | 0.3445 | 0.6505 | −0.2295 | −0.6085 | 0.0645 | −0.1185 | 0.3395 | −0.0685 |
| NotFound | −1.1135 | 0.1095 | 0.4655 | −0.6895 | −0.9215 | −0.6855 | −0.6645 | −0.9335 |
| NotFound | −0.9255 | −0.0795 | −1.0465 | −0.2125 | 0.1245 | 0.0025 | −0.5445 | 0.5605 |
| NotFound | 0.7995 | −0.0715 | −0.0185 | 0.9875 | 0.5075 | −0.2005 | 0.5435 | −0.0075 |
| NotFound | −0.033 | 0.33 | 0.425 | −1.54 | −0.142 | −1.05 | −0.918 | −0.418 |
| NotFound | −0.724 | −0.085 | 0.424 | 0.107 | 0.723 | −1.197 | 0.029 | 0.553 |
| NotFound | 0.4215 | 0.9395 | 2.2245 | 0.1375 | 1.5815 | 0.1875 | 0.6415 | −0.4975 |
| NotFound | 1.2515 | 0.7625 | −0.1525 | −0.1525 | −0.5615 | 0.3905 | 0.7015 | 0.2925 |
| NotFound | 0.5065 | 0.3185 | 0.6295 | −1.1685 | 1.0215 | 1.2375 | 1.5995 | 0.2455 |
| NotFound | −0.4785 | −0.5625 | −0.3095 | 1.0015 | 0.3155 | −0.7075 | 0.1735 | −0.7445 |
| NotFound | 0.026 | −2.851 | −3.57 | −2.291 | −0.228 | −2.283 | −3.227 | −1.271 |
| NotFound | −0.571 | −0.911 | −0.752 | −1.162 | −0.877 | 0.273 | −1.03 | 2.517 |
| NotFound | −0.0325 | 0.1155 | 0.0195 | 0.4205 | 0.6215 | 0.2365 | 0.3415 | −0.0016 |
| NotFound | 1.371 | 3.857 | 0.301 | 0.207 | −0.142 | 1.128 | 3.405 | 1.404 |
| NotFound | −0.08 | 0.209 | 0.087 | 0.406 | 1.124 | 0.453 | −0.127 | 0.128 |
| NotFound | 1.1155 | −0.0025 | 2.6955 | −0.3675 | −0.7635 | −0.5425 | −0.3555 | 0.3455 |
| NotFound | −0.237 | −0.179 | 0.034 | −0.119 | 0.223 | −0.117 | 0.102 | 0.021 |
| NotFound | 0.839 | 1.562 | 0.45 | 2.683 | 1.814 | 2.831 | 2.335 | 0.831 |
| NotFound | −0.5225 | 0.9685 | 0.9395 | 0.9325 | 2.0205 | 0.9825 | 0.7285 | 0.9355 |
| NotFound | 0.355 | −0.421 | 0.034 | 0.654 | 0.487 | 0.583 | −0.055 | 0.413 |
| NotFound | −0.0135 | 0.2645 | 0.3755 | −0.2175 | −0.2655 | −0.4305 | 0.4545 | 0.3425 |
| NotFound | −0.1405 | 0.2425 | 0.1585 | 0.4205 | −0.2985 | −0.1995 | 0.3005 | −0.2885 |
| NotFound | 0.821 | 0.353 | 0.052 | 1.013 | 1.858 | 1.128 | 1.578 | 0.981 |
| NotFound | 0.5035 | 0.1305 | −0.0345 | −0.3875 | −0.3515 | 0.1915 | −0.0695 | −0.0435 |
| NotFound | −0.06 | 0.392 | 0.358 | 0.023 | −0.418 | 0.578 | 1.131 | 0.509 |
| NotFound | 1.537 | 0.596 | −0.24 | −0.997 | 0.157 | 0.741 | −0.209 | 1.138 |
| NPDC1 | −0.8305 | −0.2155 | 0.1715 | −0.1355 | −0.7285 | 0.1835 | 0.1925 | 0.2745 |
| NPEPPS | 0.718 | 1.302 | −0.634 | −0.302 | 0.839 | 1.262 | 1.004 | −0.317 |
| NPY | −0.078 | −0.586 | −0.006 | −0.506 | 0.291 | 1.155 | 0.604 | 0.234 |
| NQO3A2 | 0.314 | −1.012 | −0.459 | 2.3 | 1.931 | 1.648 | 0.871 | 1.251 |
| NRAS | −0.028 | 0.89 | −0.474 | −0.558 | −1.842 | −0.987 | −3.542 | −1.328 |
| NRIP1 | −0.2085 | 0.6915 | −1.0745 | 2.1585 | 1.4315 | 1.5505 | 0.7855 | 0.9025 |
| NTN4 | −0.0615 | −0.3845 | −0.1915 | 0.1915 | −0.0325 | 0.1945 | 0.0135 | −0.4315 |
| NUCB2 | −0.4255 | −0.0915 | −1.3075 | −0.6945 | −0.0265 | 0.1525 | −0.5855 | 0.4885 |
| NUDT5 | −0.244 | 0.139 | 0.227 | 0.21 | 0.804 | 0.707 | −0.813 | 0.68 |
| NUDT6 | −0.469 | −0.633 | 0.234 | −0.617 | −0.179 | −0.756 | −0.043 | 0.054 |
| OCIAD2 | −0.2045 | −0.0875 | −1.2675 | −0.1075 | 0.3985 | −0.2095 | −1.1085 | 0.0795 |
| OFD1 | −0.3535 | −1.3085 | −0.1665 | 0.9055 | −0.9225 | 0.1345 | −0.2415 | 0.3275 |
| OGFRL1 | −0.083 | 2.87 | 1.515 | −0.068 | 0.402 | 0.318 | 0.172 | −0.22 |
| OPLAH | −0.1035 | 0.6405 | 0.0735 | −0.1775 | 0.2295 | −1.1825 | −0.3425 | −0.2775 |
| ORMDL3 | 1.475 | 0.038 | −0.996 | 2.373 | 2.618 | −0.128 | −1.829 | 2.691 |
| OSBPL3 | −0.272 | −0.556 | 0.342 | 0.171 | 0.352 | 1.035 | 0.482 | 0.47 |
| OSR2 | −0.1875 | 0.6025 | −0.0775 | 0.5325 | 1.6175 | −0.0035 | 0.9625 | 0.3065 |
| OSTF1 | 0.599 | 0.981 | 1.093 | −0.452 | −1.961 | −0.114 | −0.06 | 1.856 |
| P2RX1 | −0.5295 | 1.3935 | −0.4385 | 1.6425 | 0.8075 | −0.2005 | 1.4955 | 0.2245 |
| P2RY2 | 0.018 | 0.289 | 0.474 | −0.7 | 0.204 | −0.413 | −0.552 | 0.105 |
| PACAP | 0.235 | 0.402 | −0.316 | 0.767 | 1.676 | −0.159 | 1.273 | 0.543 |
| PADI2 | −0.4785 | −0.5755 | −0.8555 | 1.0325 | 0.4865 | −0.7495 | −1.4515 | −1.1835 |
| PAPD5 | 0.65 | 0.85 | 0.337 | 0.727 | 0.75 | 0.256 | 0.19 | 0.59 |
| PAPSS2 | 0.681 | 0.56 | −0.378 | −0.247 | −0.165 | 0.431 | −0.378 | 0.343 |
| PARD8A | −0.618 | −0.267 | 0.471 | 0.107 | 1.097 | −0.104 | −0.6 | −0.834 |
| PARP1 | −0.304 | −0.284 | −0.404 | −0.743 | 0.487 | 0.121 | −0.095 | 0.412 |
| PARP9 | 0.5016 | −1.4225 | −0.3415 | 1.3625 | 0.0835 | −0.3785 | −0.2655 | −0.6175 |
| PAXIP1L | 0.1635 | 1.1455 | 1.1385 | 0.5505 | 0.3535 | 0.3565 | −0.3505 | −0.1875 |
| PBX3 | −0.2305 | 0.6465 | 1.7235 | 0.0505 | 0.3575 | 1.0785 | −0.4295 | 0.5595 |
| PCDHB11 | −1.227 | 3.065 | 1.888 | 0.919 | −1.425 | 2.217 | −0.097 | −1.088 |
| PCDHB16 | 0.885 | −0.191 | −0.971 | 0.1 | 1.071 | 0.047 | −0.736 | −0.328 |
| PCDHB2 | 0.3025 | 0.1695 | 0.5505 | 0.8985 | 1.3045 | 0.7305 | −0.3725 | 1.0805 |
| PCM1 | −0.3575 | 0.2555 | 0.1025 | 0.3885 | 0.9955 | 0.6055 | 0.9795 | 0.6895 |
| PCNA | 0.137 | 0.287 | 0.011 | 0.216 | 0.197 | 0.259 | 0.264 | −0.212 |
| PDC01 | 0.7525 | −1.2205 | −0.2685 | −2.0085 | −0.3335 | −1.6535 | −0.5085 | −0.7365 |
| PDC010 | −0.249 | 0.212 | −0.004 | 0.076 | −0.363 | 0.006 | 0.077 | 0.134 |
| PDCD4 | −0.2545 | 1.0385 | 0.3585 | 0.9975 | −0.1516 | −0.0325 | 0.1685 | 0.6985 |
| PDCD5 | −0.4145 | 0.5165 | 0.9425 | 0.4965 | −1.4285 | 0.7985 | −1.6525 | −0.7635 |
| PDCD8 | −0.5945 | 0.5815 | 0.1575 | −0.7635 | −0.5345 | −0.2745 | 0.1015 | −0.1475 |
| PDE4DIP | −0.1105 | 0.5005 | 1.1345 | −0.5375 | 0.3815 | 0.0885 | −0.4935 | −0.0755 |
| PDHA1 | −2.293 | −1.148 | 0.308 | 0.034 | 0.709 | 1.239 | −2.092 | −0.816 |
| PECI | −0.4735 | −0.1605 | 0.0525 | −0.4715 | −0.2005 | 0.9425 | 0.5185 | 0.4865 |
| PEG10 | −1.5845 | 0.0685 | 0.0555 | −0.5815 | −1.5315 | −0.8215 | −1.2705 | −0.0495 |
| PER2 | −0.558 | 0.742 | −0.219 | −0.185 | 0.329 | −0.349 | 0.205 | −0.11 |
| PERP | −0.8995 | −0.2005 | −0.8395 | −1.5785 | −0.7835 | 0.2115 | 0.0785 | −0.6275 |
| PEX1 | 0.277 | −0.016 | −0.16 | −0.08 | −0.16 | 0.141 | −1.106 | −0.044 |
| PFAAP5 | −0.6845 | −1.6645 | 0.6305 | −0.2265 | −1.3145 | −1.0515 | −0.9415 | 0.1045 |
| PFDN2 | −0.126 | 0.859 | 0.547 | −0.129 | 0.25 | 0.764 | 0.506 | −0.048 |
| PGM1 | 0.1725 | −0.0235 | 0.0215 | 0.3105 | 0.1775 | 0.1375 | −0.1935 | 0.3405 |
| PGR1 | −1.2915 | −0.1265 | 0.7985 | −0.5215 | −1.5985 | −1.1585 | −0.6115 | −0.9705 |
| PHAX | −0.419 | 1.143 | −0.564 | 0.583 | −0.145 | −0.472 | −0.023 | −0.124 |
| PHGDH | 0.7815 | 1.3775 | −0.8295 | −0.9865 | −0.2785 | −1.1195 | 0.2775 | −0.8325 |
| PHYH | 0.7665 | 0.2945 | −0.9375 | −0.0215 | 0.6945 | 0.7755 | 1.0265 | 0.0585 |
| PI85PA | −0.0025 | 0.7285 | −0.3085 | 0.4335 | 1.9035 | 0.2385 | 1.2805 | 0.7145 |
| PIGT | 0.614 | 3.165 | 0.402 | −0.241 | 1.18 | 0.584 | 1.158 | −0.177 |
| PIM2 | 0.0705 | 0.9615 | 0.3365 | −0.5195 | 0.7945 | 0.7415 | −0.2585 | 0.2425 |
| PISD | −0.654 | −0.319 | −0.212 | 0.851 | 2.284 | 2.547 | 0.126 | 0.789 |
| PITPNC1 | 0.2885 | 0.9005 | 0.5325 | 0.9075 | 0.7785 | 0.5295 | 0.3885 | 1.2955 |
| PKIB | −0.503 | 0.478 | 0.333 | 0.387 | −0.738 | 0.717 | −0.142 | 0.564 |
| PKMYT1 | −1.027 | −0.17 | −0.137 | 0.229 | 1.660 | 0.754 | 1.01 | 0.785 |
| PLA2G4B | 0.7655 | 1.1735 | −0.2965 | 2.2265 | −0.4455 | −0.0845 | 0.6245 | 0.2225 |
| PLAC8 | −0.729 | 0.31 | 0.35 | 0.577 | 0.613 | 0.004 | 0.916 | −0.16 |
| PLCB1 | 0.6595 | 0.4875 | −0.2985 | 0.0515 | −2.0205 | 0.0075 | −1.0275 | −0.4395 |
| PLCXD1 | −0.195 | −0.243 | −0.704 | −0.235 | −0.928 | −0.719 | −0.399 | 0.085 |
| PLEK2 | −0.1435 | −0.1525 | −0.5245 | −0.0505 | −0.6165 | −0.1365 | −0.2045 | 0.0175 |
| PLO0 | 0.3155 | 0.6525 | −0.1145 | −0.4495 | 0.0035 | 0.2325 | 0.7905 | −0.3875 |
| PNAS-4 | −0.3035 | 1.9235 | −0.2175 | −0.3205 | −0.4835 | −0.1985 | −1.2765 | −0.2885 |
| PNLIPRP2 | 0.3525 | 0.5275 | 1.1825 | −0.7335 | −0.8205 | −0.3065 | −0.1705 | −0.0195 |
| PNMA3 | −0.0995 | −0.7365 | −0.1615 | 0.0185 | −0.1345 | −0.2425 | 0.1005 | −0.8215 |
| POLB | 0.965 | 0.595 | 0.138 | −0.111 | −0.006 | 0.223 | 0.183 | 0.212 |
| POLR2F | 0.0015 | 0.5325 | −0.4685 | 0.6895 | 0.2195 | −0.4095 | −0.2995 | −0.2275 |
| POLR2H | 0.3915 | 0.7825 | −0.0735 | 0.4285 | 0.2545 | −0.4385 | −0.3005 | 0.1315 |
| POMC | −0.573 | 0.602 | 0.285 | 0.114 | −0.267 | 0.639 | 0.071 | 0.004 |
| POP4 | −0.621 | 1 | 0.037 | −0.257 | −1.451 | −1.105 | −0.206 | −0.388 |
| POP7 | 0.475 | 2.023 | 0.121 | 0.307 | 0.633 | 0.188 | 1.965 | 0.589 |
| PP1057 | 0.2995 | 0.4225 | 0.6725 | −0.3618 | 0.3355 | −0.0985 | −0.3485 | 0.0855 |
| PPFIA1 | 0.52 | 0.265 | −0.34 | 0.256 | 0.381 | 0.317 | 1.104 | −0.552 |
| PPFIBP2 | 0.4345 | 0.3165 | 0.3025 | −0.5215 | −0.1745 | −0.2915 | −0.3305 | 0.4355 |
| PPGB | 0.2145 | −0.4935 | 0.3565 | −0.6245 | −0.0925 | 0.0315 | 0.4745 | 0.1605 |
| PPHLN1 | 0.5625 | 0.4585 | 0.0045 | 0.3965 | 0.5825 | 0.5695 | 0.8395 | 0.0835 |
| PPIF | 0.3985 | −0.0535 | −0.0695 | −0.7715 | −0.4985 | −0.2025 | −1.0575 | 0.2255 |
| PPIL1 | −0.5505 | 0.5865 | −0.4835 | −0.3595 | −0.7945 | 0.2235 | 0.4455 | 0.4095 |
| PPOX | 0.849 | 2.383 | 1.968 | −1.229 | 0.277 | −1.062 | −1.099 | 2.575 |
| PPP1R16 [symbol] | −0.149 | 0.681 | 0.204 | 0.099 | 0.144 | 0.118 | 0.32 | 0.034 |
| PPP1R3C | −0.3 | 0.348 | −0.788 | 0.002 | 0.759 | 1.351 | 0.732 | 0.841 |
| PPP2R2D | −0.9645 | −0.3075 | 0.1975 | 4.6335 | −0.6075 | −0.1645 | 4.4815 | 1.1645 |
| PPP2R5A | −0.134 | 0.687 | 0.994 | 0.316 | 0.685 | 0.448 | 1.237 | 0.341 |
| PRAME | 0.4875 | 0.5215 | −0.1095 | −0.3115 | 1.0955 | −0.2335 | −0.3715 | 1.1315 |
| PRC1 | −0.872 | −0.255 | 0.393 | 0.91 | 1.006 | 0.447 | 0.28 | −0.045 |
| PRIM2A | −0.0935 | 0.1885 | −0.7485 | −0.2385 | 1.6935 | 1.2625 | 0.8715 | 0.8435 |
| PRKACB | 0.856 | 0 | −0.151 | 0.059 | 0.075 | −0.104 | −0.258 | −0.047 |
| PRKCB1 | 0.216 | −0.153 | 0.467 | −0.865 | −0.915 | −0.227 | −0.609 | −0.238 |
| PRKCI | 0.552 | −0.203 | −0.326 | −0.43 | −0.244 | −0.618 | −0.094 | −0.195 |
| PRKRIR | −0.0055 | 0.7315 | 0.8175 | −0.5435 | −0.0025 | −0.0155 | −0.4875 | 0.3725 |
| PRKWNK1 | −0.6305 | −0.3195 | −0.9575 | 0.1105 | −0.7845 | −0.3255 | −1.9235 | −0.4635 |
| PRKX | −0.172 | 0.455 | 0.084 | 0.594 | 1.631 | 0.48 | 0.893 | −0.001 |
| PRNP | −0.4595 | 0.2185 | −0.6285 | −0.0985 | −0.0475 | −0.1455 | −0.1825 | −0.2025 |
| PROL5 | 0.5545 | 0.0925 | 0.0345 | 1.0335 | 0.4775 | 0.1615 | 1.5375 | −0.0525 |
| PRPF18 | −0.4665 | −0.7355 | −0.0415 | −1.1215 | −0.5385 | −0.2735 | −1.1095 | −0.2625 |
| PRPSAP1 | −1.4855 | −0.1885 | −0.5515 | −1.5635 | −0.7825 | −0.3595 | −0.3115 | −0.1305 |
| PRSS12 | 1.5935 | 1.0365 | −1.4485 | 1.9095 | 1.6495 | 0.7505 | −1.6465 | −0.7115 |
| PRSS16 | −0.1485 | 0.2845 | 0.1095 | 0.3965 | 0.8045 | 0.1835 | 0.1775 | −0.2445 |
| PSD3 | 0.5535 | 0.5965 | 0.2075 | −0.1785 | −0.3325 | 0.1165 | 0.2755 | 0.3435 |
| PSMA3 | 0.514 | 0.418 | 0.629 | 0.95 | −0.082 | 0.722 | 0.886 | −0.211 |
| PSMA7 | 0.082 | 0.391 | 0.006 | 0.69 | 1.131 | 0.47 | 0.495 | 0.097 |
| PSMC5 | 0.1855 | 1.7485 | 0.4895 | 1.2545 | 1.4885 | 1.5685 | 1.0085 | 0.9345 |
| PSMC6 | 0.407 | 0.281 | 0.495 | 1.033 | 1.294 | 2.235 | 0.898 | −0.244 |
| PSMD12 | 0.8485 | 3.6385 | 2.8915 | −0.3225 | 0.8615 | 0.1745 | −0.4755 | 0.2215 |
| PSMD12 | −2.5055 | −1.1885 | −0.8395 | 0.8095 | 0.2555 | 0.3255 | 1.1305 | 0.6275 |
| PSMD3 | −0.1825 | −0.5875 | −1.1055 | −0.6375 | −0.4065 | −0.8575 | −0.4135 | 0.7315 |
| PSPH | −0.45 | −0.696 | −0.188 | 0.636 | 0.948 | 0.082 | −0.137 | −0.428 |
| PTD012 | −0.202 | 3.206 | −0.019 | 0.013 | −0.406 | −0.027 | −0.748 | 0.116 |
| PTGER3 | −0.121 | 1.43 | 0.472 | 0.715 | −0.561 | 0.212 | −0.968 | 1.215 |
| PTHLH | −0.3685 | −0.4065 | −0.1165 | −0.3875 | 1.0455 | −0.1745 | −0.2285 | 0.4055 |
| PTK6 | 0.291 | −0.188 | 0.121 | 0.075 | 0.503 | 0.193 | 0.578 | −0.131 |
| PTPN4 | −0.684 | 0.275 | −0.131 | 0.715 | −0.473 | −0.647 | 0.003 | 0.053 |
| PTPN7 | −0.434 | 0.048 | 1.162 | −0.524 | 2.199 | 0.854 | 0.429 | 0.985 |
| PTPRF | 2.553 | −0.176 | −0.276 | −0.603 | −0.441 | 0.609 | −0.207 | 0.065 |
| PTPRN2 | −0.6885 | −0.0785 | −0.0175 | −0.6755 | −0.3265 | −0.8385 | −0.7175 | −0.2315 |
| PTPRT | 0.1685 | 0.4665 | 0.8765 | 0.6585 | 0.5455 | −0.1375 | −0.0085 | 0.3745 |
| PTS | 0.972 | −0.181 | −0.429 | −0.498 | 0.147 | 0.558 | −0.413 | 0.092 |
| PTTG1 | 0.1815 | 0.6245 | −0.0955 | −0.9416 | −0.0435 | 0.2525 | 0.2025 | 0.8425 |
| PURB | 0.6785 | 0.2805 | 0.5225 | −0.3315 | 0.2935 | 1.0285 | −1.3285 | −0.1325 |
| PXMP2 | −0.3445 | −0.0385 | 0.1585 | 1.5695 | 0.2055 | −0.7615 | −0.8035 | 0.5725 |
| PYCARD | 1.346 | −0.804 | 1.839 | 0.874 | −1.84 | 0.161 | −2.103 | −0.291 |
| PYCR1 | 0.475 | −0.897 | −0.224 | −0.455 | −0.682 | −0.932 | 0.979 | −0.798 |
| QDPR | 0.8735 | 1.4785 | −0.8825 | −1.1265 | 0.5955 | 0.0255 | 0.3175 | 0.1855 |
| QPRT | −0.551 | 0.271 | 0.913 | 0.151 | 0.648 | 1.055 | 1.948 | 0.182 |
| QSCN9 | 0.5705 | 1.8095 | −0.4145 | −1.0395 | 0.0365 | 1.7355 | −0.0575 | 0.0635 |
| RAB11FIP | −0.242 | −0.042 | −0.103 | −0.301 | −0.755 | −0.457 | −0.745 | 0.189 |
| RAB11FIP | −0.432 | −0.018 | 1.435 | 0.291 | 0.424 | 0.126 | −0.824 | −0.208 |
| RAB13 | 1.886 | −0.25 | 0.294 | −0.077 | 0.783 | 1.02 | 1.033 | −0.282 |
| RAB27A | 0.594 | 0.489 | −1.504 | −0.969 | −0.178 | 0.79 | 0.857 | −1.252 |
| RAB30 | 0.3355 | 1.2085 | 0.0045 | 0.2305 | 0.1765 | −0.0315 | −0.1375 | 0.6005 |
| RAB34 | −0.833 | −1.226 | 1.281 | −0.015 | 1.824 | 0.168 | 0.681 | 1.07 |
| RAB35 | −0.834 | 0.053 | 0.192 | −0.333 | −0.116 | −0.108 | 0.338 | 0.127 |
| RAB39B | −0.1275 | 0.8875 | −0.1795 | 2.0325 | 0.6845 | −0.0515 | −0.6755 | 0.0205 |
| RAB3A | 2.064 | 1.421 | −0.364 | −0.303 | 2.285 | 2.217 | −0.848 | 1.483 |
| RAB3IP | −0.501 | 0.23 | −0.78 | 0.089 | 0.152 | −0.164 | −0.646 | 0.127 |
| RABEP1 | 0.702 | 0.358 | 0.763 | 0.096 | 1.248 | 0.078 | 0.887 | 1.479 |
| RABGAP1 | 0.8175 | −0.7155 | −0.7095 | −0.2925 | 0.4365 | 0.0895 | 0.5385 | 0.3495 |
| RACGAP1 | −0.6695 | 0.1845 | 0.8745 | 0.1315 | 0.3905 | 0.2625 | 0.3385 | 1.2215 |
| RAD17 | −0.066 | 0.188 | 0.336 | 0.132 | 0.589 | 0.129 | 0.495 | 0.116 |
| RAD51 | −0.123 | −0.221 | 0.03 | −0.048 | 0.207 | −0.031 | 0.168 | −0.08 |
| RAD51AP | −0.2225 | 0.1035 | 0.8555 | −0.3715 | 0.1535 | 0.2165 | 0.7865 | 0.5785 |
| RAD52 | 0.916 | 1.404 | 0.834 | 0.587 | 1.053 | 0.435 | 0.32 | 1.744 |
| RAD54L | 0.0515 | 1.4965 | 0.0305 | −0.4065 | −0.6205 | −0.3975 | 0.1485 | −0.2715 |
| RAMP | 1.4725 | −0.6815 | −1.5675 | 0.7635 | 1.2775 | −1.1425 | −1.0705 | −0.7535 |
| RANBP1 | 0.3505 | 0.2935 | 0.1115 | −0.5645 | 0.2425 | −0.0175 | −0.3535 | 0.3275 |
| RARRES1 | 0.208 | −0.778 | 0.051 | −0.897 | 0.105 | −0.029 | −1.247 | −0.032 |
| RBBP7 | −0.0245 | 0.1495 | −0.3335 | −0.1855 | 0.0095 | 0.5435 | −0.3905 | −0.0245 |
| RBBP8 | −0.0325 | −0.1895 | −0.1885 | 0.3785 | −0.6575 | −0.1315 | 0.2785 | −0.0775 |
| RBM8A | −0.833 | −0.569 | 0.6 | −0.104 | −0.841 | 0.333 | −0.662 | 1.325 |
| RBX1 | 0.224 | −0.289 | 0.034 | 0.31 | 0.104 | 0.269 | −0.316 | −0.529 |
| RDHE2 | −0.56 | −0.072 | 0.291 | 0.031 | 0.705 | 0.482 | 1.172 | 0.083 |
| REA | −0.742 | 0.108 | 0.044 | 0.081 | −0.234 | −0.138 | 0.129 | 0.008 |
| REC14 | −0.4085 | 0.7415 | −0.6365 | −0.0895 | 0.1925 | 0.3795 | −0.8615 | −0.0145 |
| REG1A | −0.808 | −0.52 | 0.222 | −1.083 | −0.001 | 0.137 | 0.874 | 0.26 |
| RELB | 1.689 | 0.994 | 0.446 | 1.808 | 2.338 | 0.457 | −0.288 | 0.604 |
| REPIN1 | 1.265 | −0.686 | −0.483 | 1.112 | 1.381 | 1.15 | 0.813 | −0.211 |
| REPS2 | 0.6325 | −0.3095 | 0.8535 | −0.1695 | 0.3595 | −0.3155 | −0.5595 | 0.3195 |
| RERG | −0.2915 | 0.3135 | −0.7265 | −0.3365 | −0.8355 | 0.5125 | −0.3325 | −0.6165 |
| RFC4 | −0.8155 | −1.2005 | −0.4885 | −0.8745 | −1.8385 | −1.9145 | −0.7895 | 0.4535 |
| RGS10 | 1.1295 | 1.4935 | 1.1735 | 1.5805 | 1.9275 | 0.2805 | 0.1835 | 0.0645 |
| RH8DL2 | 0.43 | −0.547 | −0.297 | −0.005 | −0.447 | 0.441 | 0.344 | 0.102 |
| RIBC2 | −0.088 | 0.06 | −0.504 | −0.399 | 0.217 | −0.255 | 0.337 | −0.219 |
| RIC-8 | 0.145 | 0.296 | −0.593 | −0.342 | 0.852 | 0.815 | −0.345 | 0.665 |
| RIT1 | 0.262 | 0.952 | 0.287 | 0.136 | −0.265 | −0.04 | −0.374 | 0.354 |
| RLN2 | 0.517 | −0.3 | −0.575 | −0.476 | −0.247 | 0.885 | −0.479 | 0.111 |
| RNF10 | −0.0935 | −0.0755 | 0.0375 | −0.5645 | −0.0375 | 0.2425 | 0.5095 | −0.3785 |
| RNF135 | −1.1115 | −0.3505 | −0.7505 | −1.3925 | −0.9975 | −0.5475 | −0.3155 | 1.0685 |
| RNF7 | 0.88 | −0.578 | 0.577 | 0.745 | 0.648 | 0.265 | −0.505 | 0.194 |
| ROPN1 | 1.0665 | 1.0785 | −1.1316 | −1.5585 | −1.7375 | −0.1305 | −1.0285 | −0.7905 |
| RPA3 | −0.5195 | −1.0485 | −0.7315 | −0.3575 | −0.2915 | −0.0565 | −0.2775 | −0.3735 |
| RPESP | 0.277 | −0.606 | −0.428 | −0.712 | −0.738 | −0.303 | −0.154 | 0.541 |
| RPL11 | 0.132 | 0.304 | −0.562 | 0.049 | −0.328 | −0.63 | −0.425 | 0.002 |
| RPL13 | −0.78 | −0.141 | 0.169 | −0.559 | −0.462 | 0.556 | 1.002 | −0.034 |
| RPL13A | −0.0905 | −0.2385 | 0.0065 | 0.3005 | −0.2365 | −0.5145 | −0.2365 | −0.0885 |
| RPL15 | 0.089 | 2.661 | 1.451 | 1.375 | 1.432 | −0.418 | −0.743 | 0.527 |
| RPL18A | −0.911 | −0.817 | −0.818 | −0.536 | −0.479 | −0.35 | −0.359 | −0.678 |
| RPL19 | 0.287 | 0.896 | −0.164 | −0.918 | −0.481 | 0.394 | 0.623 | 0.205 |
| RPL21 | 0.1085 | 0.5225 | −0.0275 | −0.4595 | −0.3915 | 0.4295 | 0.4915 | −0.0415 |
| RPL23A | −0.278 | −0.592 | −0.527 | −0.781 | −0.045 | −0.169 | −0.438 | −0.498 |
| RPL23A | −0.1295 | −0.6405 | 0.2725 | −0.0375 | 0.0375 | 0.1385 | −0.6835 | −0.2495 |
| RPL26L1 | 0.2405 | −0.0765 | −0.1975 | −0.0125 | −0.1485 | 0.0515 | −0.1775 | 0.0795 |
| RPL29 | −0.1595 | −0.1965 | −0.3575 | 1.2275 | 0.4275 | 0.1775 | −0.3085 | 0.0115 |
| RPL35 | 0.1975 | −0.7775 | −0.8475 | −0.5295 | −1.0595 | −0.7695 | −0.5315 | 0.1065 |
| RPL35AL | −0.2835 | −0.3085 | 0.4995 | −0.7125 | −0.5485 | 0.1015 | 0.2595 | 0.1765 |
| RPS16 | −0.4585 | −0.5465 | −0.3455 | −0.5835 | −0.2365 | 0.3595 | −0.9115 | 0.0805 |
| RPS4Y1 | 0.6605 | −0.2785 | −0.4325 | 0.0855 | −0.5045 | −0.7695 | −0.2655 | −0.2705 |
| RRAGA | 0.908 | −0.417 | −0.101 | −0.095 | 0.863 | 0.385 | 0.59 | 0.753 |
| RRAGD | 0.044 | 0.191 | 2.006 | 0.678 | 0.471 | 0.157 | 1.152 | 0.04 |
| RRM1 | −0.013 | −0.002 | −0.177 | 1.159 | −0.753 | −0.094 | −0.253 | 0.251 |
| RRM2 | −6.633 | −1.77 | −1.786 | −1.202 | −0.451 | 0.007 | −1.265 | −1.037 |
| RSAFD1 | −0.2485 | −0.0355 | −0.0055 | 0.0115 | −0.3485 | −0.0445 | −0.1115 | 0.0055 |
| RSNL2 | −0.2415 | 0.7275 | 2.3205 | −0.0655 | −0.4755 | −0.9725 | −0.0875 | −0.3145 |
| RSU1 | 0.426 | 0.06 | 0.187 | 0.321 | −0.556 | −0.602 | −0.906 | −0.923 |
| RTN4IP1 | −2.062 | 1.991 | 1.325 | 0.944 | 1.102 | 0.115 | −1.004 | 0.911 |
| S100A11 | −1.522 | 1.636 | 1.879 | 0.862 | 0.641 | −0.08 | −2.135 | 1.348 |
| S100A14 | 0.6325 | 0.8785 | −1.0115 | −1.2355 | −0.8105 | −1.1925 | −1.7475 | −0.4765 |
| S100A16 | 0.2225 | 0.2275 | 0.3665 | 0.4825 | −0.4915 | 0.0835 | 0.5555 | 0.3805 |
| S100A9 | 1.49 | 0.084 | −0.584 | −1.231 | −0.158 | −0.172 | −0.108 | −0.286 |
| SAE1 | 0.2215 | −0.0405 | −0.6475 | 0.8585 | 0.5645 | 0.5755 | −0.3775 | −0.2225 |
| SALL2 | 0 | 0.268 | 0.477 | 0.803 | 0.357 | 0.491 | 0.544 | 0.283 |
| SARA1 | −0.0825 | 0.3095 | 0.3425 | 0.0425 | −0.3965 | 0.5485 | 0.2185 | −0.4735 |
| SARA2 | 0.396 | 2.202 | 1.783 | 0.321 | −0.389 | −0.23 | −0.561 | −0.481 |
| SAS | −0.736 | 0.518 | 1.494 | 0.555 | 1.162 | −0.809 | −0.408 | −0.922 |
| SCIN | −0.5125 | −1.6395 | −0.7355 | −0.6705 | 0.6555 | −0.7355 | −1.5365 | −0.6755 |
| SCML1 | −0.51 | 1.053 | −0.134 | 0.268 | −0.598 | 0.061 | −0.437 | 0.931 |
| SCPEP1 | −0.1645 | 0.3265 | 0.1805 | 0.5995 | −2.3055 | −1.4105 | 0.3495 | −0.9775 |
| SCRIB | −0.26 | 0.287 | −0.073 | −0.007 | −0.302 | −0.464 | 0.144 | 0.313 |
| SDC1 | −0.249 | −0.636 | −0.355 | −0.029 | −0.317 | −0.85 | −0.041 | −0.2 |
| SDC3 | 0.2885 | 0.8585 | −1.2165 | 0.2785 | 0.1595 | −0.0665 | 0.3535 | 0.3685 |
| SDCCAG0 | −0.2745 | −0.1055 | −0.1305 | 0.4625 | −0.2065 | −0.1495 | −0.1925 | 0.7325 |
| SEC61G | 0.552 | 0.783 | 0.177 | 0.083 | −0.133 | −0.143 | −1.499 | 0.789 |
| SECP43 | 0.8845 | −0.3875 | 0.3985 | 0.8635 | 1.7535 | 1.3445 | 0.8965 | 1.0935 |
| SEDLP | −1.292 | 0.88 | 0.313 | 0.081 | −0.892 | 0.976 | 0.755 | 0.824 |
| SEMA3B | −0.2555 | 0.6675 | −1.5615 | 1.5425 | 0.0415 | −0.2515 | 0.8945 | −0.0275 |
| SEMA3C | −0.9995 | 0.2995 | −0.0855 | 0.5175 | 1.3245 | 0.9655 | −0.2465 | 0.8255 |
| SEPW1 | 3.17 | 1.535 | −1.048 | 1.817 | 4.11 | 3.028 | 1.859 | 1.815 |
| SERP1 | −0.5865 | −0.8845 | 0.0005 | −0.8035 | −1.6665 | −0.9995 | −0.0485 | −0.6825 |
| SERPINA [symbol] | −2.029 | −1.94 | −1.507 | −1.75 | −2.155 | −1.163 | −1.322 | 0.602 |
| SERPINB [symbol] | −0.165 | 0.468 | 0.396 | −0.14 | −0.249 | 0.018 | 0.188 | 0.402 |
| SFRP1 | 0.222 | −1.331 | −0.02 | 0.863 | −0.964 | −0.081 | −0.312 | 0.106 |
| SFRS6 | −0.8615 | −1.4965 | −0.4815 | −0.3065 | −0.2145 | 1.4525 | −1.0155 | −0.6005 |
| SGCD | 1.3525 | 1.7945 | 1.1625 | 2.2505 | −0.7625 | −0.9245 | −2.0595 | −1.4955 |
| SGCE | −0.643 | −0.022 | −0.455 | −0.178 | 0.259 | −0.261 | −0.201 | −0.131 |
| SH3BGRL | 0.875 | 1.947 | 0.901 | −0.117 | 1.974 | 0.89 | 2.079 | 1.751 |
| SHFM1 | 0.1855 | 0.6195 | −0.1455 | 0.2525 | 0.1975 | 0.0195 | 0.1375 | 0.1665 |
| SIAT7B | 0.179 | 0.278 | 0.277 | 0.614 | 1.726 | 0.853 | 0.767 | 0.737 |
| SIGLEC12 | 0.588 | −0.294 | −0.044 | 0.057 | −0.749 | −0.464 | −0.397 | −0.288 |
| SIT | 0.2955 | −0.1525 | 0.1105 | 0.0045 | 0.0615 | −0.0045 | 0.8175 | −0.4055 |
| SITPEC | −0.2255 | 0.4295 | −0.2545 | 6.2595 | −0.1905 | 0.1525 | −0.2505 | −0.0685 |
| SKP1A | −1.0115 | 2.1825 | −1.1685 | −0.9545 | −0.9285 | −0.8135 | −1.1855 | 0.9595 |
| SLC12A9 | 3.498 | 1.72 | −0.358 | 0.362 | 1.06 | 0.596 | 3.896 | 1.133 |
| SLC13A3 | 1.2575 | 0.7445 | 0.9425 | 1.4495 | 1.2825 | 2.6315 | 0.7265 | 3.4025 |
| SLC16A6 | 2.509 | 0.438 | 0.429 | 0.098 | 1.502 | 2.02 | −0.444 | −0.32 |
| SLC19A2 | 0.2525 | −0.4865 | −1.4415 | 1.2255 | 1.3125 | −0.4615 | 2.4155 | 1.1235 |
| SLC1A1 | 2.1065 | 0.0465 | −1.5105 | −1.7555 | 0.3505 | −0.6785 | −1.6735 | −0.6585 |
| SLC1A2 | 0.1275 | 0.8835 | 0.9815 | −0.0205 | 1.2475 | −0.1835 | −0.5505 | 0.5255 |
| SLC24A3 | 0.061 | 0.532 | 0.715 | 0.116 | 0.824 | 1.16 | 0.886 | 0.351 |
| SLC25A29 | −0.296 | 0.524 | 0.672 | 0.437 | −0.41 | −0.156 | −0.002 | 0.451 |
| SLC25A4 | −0.105 | 0.4 | −0.203 | 0.837 | 0.358 | −0.155 | 3.003 | 0.939 |
| SLC25A5 | 0.4605 | 0.7575 | −0.9855 | −0.9865 | 0.4455 | −0.7575 | −0.4615 | 0.2115 |
| SLC27A2 | −0.117 | 0.168 | −0.481 | 0.764 | −0.638 | 0.048 | 0.219 | −0.188 |
| SLC27A5 | −0.487 | 1.525 | −0.923 | 1.367 | −1.193 | 0.35 | 0.636 | 1.098 |
| SLC30A8 | 3.3405 | −0.8555 | −0.0825 | 2.4405 | 2.6865 | 1.3415 | 0.4565 | 2.8055 |
| SLC39A4 | 0.12 | −0.976 | 0.491 | −0.105 | 1.356 | 0.558 | −0.976 | −0.191 |
| SLC39A6 | −0.2055 | 1.0535 | −0.3315 | −0.0505 | −0.7945 | 0.0795 | 0.3255 | −0.8425 |
| SLC40A1 | −1.0175 | −0.5295 | 0.9055 | −0.4115 | −2.0925 | −0.9495 | 0.1425 | −0.4495 |
| SLC41A3 | 3.768 | −0.182 | −0.006 | −0.503 | −0.733 | −0.182 | 0.257 | −0.094 |
| SLC5A6 | 1.5215 | 0.8995 | 1.1355 | 1.3055 | 1.1435 | 1.5385 | 3.3985 | 2.2865 |
| SLC6A4 | 4.3785 | 4.1075 | −2.6135 | 3.7385 | −1.9805 | −0.5675 | −3.7945 | −0.1415 |
| SLC9A3R1 | 0.1075 | −0.5005 | −0.0385 | 1.4515 | 0.8525 | 0.2385 | 0.0875 | 0.1535 |
| SLITRK6 | 0.028 | −0.667 | −0.53 | 0.001 | 0.038 | 0.01 | −0.151 | 0.282 |
| SMA3 | −0.349 | 0.564 | −0.145 | −0.548 | −1.39 | −0.947 | −0.703 | −0.244 |
| SMAD2 | −1.035 | 0.282 | −0.898 | −0.622 | 0.06 | −0.173 | −0.773 | 0.543 |
| SMO | −0.387 | 0.142 | 1.227 | −0.247 | −0.775 | −0.16 | −0.238 | 0.698 |
| SMU1 | 1.136 | 0.842 | −0.779 | 0.085 | 0.586 | 0.268 | −0.722 | 0.266 |
| SNPH | 0.569 | 0 | −0.047 | −0.253 | −0.008 | −0.469 | −0.465 | 0.165 |
| SNRPB2 | 0.1415 | −0.0055 | −0.0615 | −0.4995 | −0.8315 | −0.4525 | 0.3175 | 1.2365 |
| SNRPD1 | −0.394 | 0.029 | −0.019 | 0.332 | 0.526 | −0.101 | −0.6 | 0.234 |
| SNRPD3 | −0.5225 | −0.5725 | −0.1345 | 0.0155 | −1.9215 | −0.1865 | −0.1495 | 0.0315 |
| SNRPG | 0.0335 | −0.1945 | −0.1995 | −0.0025 | 0.1025 | −0.5355 | −0.0155 | −0.6075 |
| SNX7 | 0.9645 | −0.0815 | −0.2615 | 0.0075 | −0.8565 | −0.3215 | −1.0615 | 0.0225 |
| SO02 | 0.0665 | 1.4725 | 0.5015 | −0.0025 | −0.1405 | −0.4815 | 0.1705 | 0.6245 |
| SORCS1 | −0.646 | −0.477 | −2.576 | −1.026 | −0.942 | −0.947 | −2.393 | −1.265 |
| SOX4 | −1.9655 | −0.2635 | 0.7625 | −1.0225 | 0.4215 | −1.4095 | 0.0125 | −0.5825 |
| SOX9 | 0.749 | 1.917 | 1.75 | −0.264 | 0.563 | 0.327 | 1.722 | 1.314 |
| SPAG18 | −0.5855 | −0.9515 | −0.3705 | −0.4585 | 0.0775 | −0.1215 | 0.2665 | −0.5405 |
| SPAG5 | −0.2065 | −0.3755 | 0.1315 | −0.1825 | −0.0645 | −0.4005 | 1.3545 | 0.7035 |
| SPC18 | −0.812 | 0.077 | 0.072 | −0.506 | −0.723 | −0.235 | −1.158 | −0.209 |
| SPFH2 | 0.95 | 0.598 | −0.142 | −0.222 | 0.479 | 0.005 | −0.31 | 0.022 |
| SPG3A | −1.215 | 0.83 | 1.85 | 0.375 | −0.805 | −0.225 | 2.475 | 1.31 |
| SPPL3 | 0.057 | −0.555 | 0.157 | 0.225 | −0.624 | 0.257 | −0.288 | 0.094 |
| SQLE | −0.467 | 1.361 | 0.605 | 0.447 | −0.087 | 1.043 | 0.279 | 0.948 |
| SQRDL | −0.9415 | −0.0085 | −0.2525 | −0.2605 | −0.0305 | 0.3415 | −0.0465 | −0.3295 |
| SREBF1 | −0.2175 | 0.2885 | −0.0875 | −0.6615 | −0.5025 | −0.2245 | 0.0215 | −0.0735 |
| SRI | −0.4205 | −0.5205 | 2.0285 | −0.3475 | −0.2755 | 0.0575 | −1.1785 | −0.3545 |
| SRPK1 | −0.2515 | 0.6395 | −0.1885 | 0.8215 | 0.0845 | 0.0285 | 0.6185 | 0.6005 |
| SSFA2 | 0.3415 | 0.3745 | 0.5855 | 0.2355 | 0.1605 | −0.1065 | −0.0335 | −0.0145 |
| SSR4 | −0.0545 | −0.6705 | −0.1705 | −0.0805 | 0.4555 | −0.5605 | 0.1395 | −0.1225 |
| STARD3NI | 0.0775 | −0.4516 | 0.7095 | 0.9845 | 1.8335 | 0.7635 | 0.4365 | −0.7025 |
| STARD7 | 0.0085 | −1.1315 | −0.3885 | −1.6625 | −0.2515 | −0.0085 | −1.0905 | −0.3085 |
| STAT1 | 0.0255 | 0.4615 | −0.0585 | 0.1765 | −0.3965 | 0.1005 | 0.3425 | 0.3125 |
| STC2 | 0.7755 | 0.2405 | 1.0655 | 0.0045 | 1.0745 | 0.0755 | 0.5535 | 0.4535 |
| STK24 | −0.0655 | −0.3325 | 0.4735 | −0.3035 | 1.3345 | 0.0305 | 0.5835 | 0.6765 |
| STK6 | −0.675 | 1.138 | 0.912 | 2.12 | −0.185 | 0.997 | 0.763 | −0.132 |
| STMN1 | 0.2385 | 0.7045 | 0.9105 | −0.9205 | −0.1175 | 1.0245 | −0.3965 | −0.0395 |
| STX1A | −0.7355 | −0.1385 | −0.8175 | 1.0025 | 0.4245 | −0.3405 | −0.5005 | −0.3495 |
| STX3A | 0.1535 | 0.8895 | −0.4345 | −0.1325 | −0.6805 | −0.3255 | −0.0995 | 0.5685 |
| STYX | 0.4525 | −0.2275 | −1.0185 | −0.4785 | −1.1195 | −0.7175 | −0.1855 | 0.3435 |
| SUGT1 | 0.308 | 0.304 | 0.188 | −0.782 | −0.008 | 0.534 | 1.054 | 0.395 |
| SUMF1 | −0.3335 | 0.6695 | 0.1405 | −0.2595 | 0.1965 | −0.1845 | −0.8235 | 0.5095 |
| SUMO2 | −0.7215 | 0.7335 | 0.0095 | 1.1585 | −0.1015 | 0.1165 | 0.5695 | 0.0865 |
| SUV39H2 | 1.9465 | −0.3425 | 0.3465 | −3.5985 | 0.3985 | 0.8475 | 1.4105 | 1.1905 |
| SYAP1 | −0.0405 | 0.1665 | −0.4545 | 0.8865 | −0.7285 | 0.6855 | 1.1835 | −0.4945 |
| SYCP2 | 0.517 | 0.17 | 0.489 | 0.399 | 0.999 | 1.254 | 0.824 | 0.957 |
| SYNGR1 | −0.0595 | 0.5015 | 0.0155 | −0.0375 | 0.5085 | −0.0465 | 0.0325 | 0.0405 |
| SYTL2 | −0.4485 | 0.0805 | −0.2255 | 0.2035 | −0.1445 | 0.3475 | −0.1205 | 0.1775 |
| TAF18 | 0.0746 | −0.6365 | −1.0005 | −1.5645 | 0.3885 | 1.0975 | 0.3645 | −0.5425 |
| TAF8 | −0.301 | 0.119 | 0.996 | −0.18 | −0.879 | −0.516 | 0.19 | 0.111 |
| TARBP1 | −1.204 | −0.215 | 0.325 | −0.106 | −0.416 | −0.494 | 0.599 | 0.045 |
| TARS | −0.0705 | 0.1555 | −0.0225 | −0.7685 | 0.1945 | 0.2225 | 0.6505 | 0.5375 |
| TATDN1 | −0.5225 | 0.3745 | −0.3135 | −0.9185 | −0.5165 | 0.0745 | 0.3245 | −0.4045 |
| TBC1D12 | 1.1375 | −0.7565 | 2.2825 | −0.7455 | 1.6525 | −0.0745 | 0.3855 | 0.2685 |
| TBC1D14 | 0.602 | −0.007 | −0.417 | 0.178 | 0.405 | 0.857 | 0.14 | 0.455 |
| TBC1D3 | 0.8485 | 2.6745 | −1.2605 | 1.3855 | −1.1565 | −1.3065 | −1.7185 | 0.0095 |
| TBCA | −0.782 | 1.163 | 1.468 | −0.045 | 6.326 | −1.367 | 0.725 | 2.042 |
| TBX1 | −0.2615 | 3.0175 | 1.2215 | 1.0485 | 1.1765 | −0.3265 | 0.0175 | −0.1645 |
| TBX3 | 0.318 | 1.291 | 0.357 | −0.198 | 1.322 | 0.085 | −0.373 | 1.015 |
| TCAP | 0.438 | 1.982 | 0.16 | 0.144 | 1.455 | 0.183 | −0.566 | 0.84 |
| TCEAL1 | −0.1185 | 1.6105 | 0.5365 | −0.6125 | 0.2065 | 0.0095 | −0.4595 | 0.8575 |
| TCEAL3 | −0.141 | 1.441 | 0.646 | −1.146 | −0.532 | −0.948 | 1.023 | 1.014 |
| TCEAL4 | 0 | 0.99 | 0.433 | 0.045 | 0.696 | 0.311 | −0.371 | −0.702 |
| TCF15 | −0.287 | −0.237 | −0.469 | 0.075 | −0.8 | −0.889 | −1.384 | −0.221 |
| TCF3 | 0.214 | −0.102 | 0.176 | 0.063 | 0.172 | 0.401 | 0.085 | 0.606 |
| TCF7L2 | −0.2185 | 0.4095 | 1.2055 | −0.5525 | 0.6595 | −0.7485 | 1.3615 | 0.3875 |
| TDRD7 | 0.9025 | −2.2395 | −4.3325 | −4.5695 | −4.0425 | −3.5575 | −4.3665 | −1.8605 |
| TESK2 | −0.774 | −2.18 | −2.232 | −0.745 | −2.076 | −1.195 | 0.122 | −0.019 |
| TFAP2B | −1.364 | 0.531 | 0.63 | −0.462 | −1.594 | −0.378 | −0.906 | −0.348 |
| TFCP2L1 | 2.4105 | 1.7255 | 1.3495 | 0.9595 | 2.4915 | 2.9675 | −0.8135 | 3.3795 |
| TFCP2L2 | 0.0125 | 3.0485 | 2.9835 | 0.3225 | 2.2405 | 2.6815 | −2.4575 | 2.6095 |
| TFF1 | 0.3235 | −0.9255 | 1.4135 | −0.7015 | 0.3755 | −0.2105 | −0.7745 | −0.7975 |
| TFF3 | −1.84 | −0.954 | −1.079 | −1.036 | 0.385 | 0.155 | −0.748 | −0.125 |
| TFRC | 0.2815 | −0.3885 | −0.2385 | −0.8605 | −0.1145 | −0.1975 | −0.6765 | −0.0455 |
| TGFBR3 | −0.137 | 0.171 | 0.185 | −0.236 | 1.071 | 0.921 | −0.299 | 0.836 |
| THY28 | 1.2085 | −1.2135 | −0.9055 | −0.3225 | −0.0535 | 0.1745 | −0.0475 | 0.0875 |
| TIF1 | 2.61 | −0.403 | 0.176 | −0.211 | 0.169 | 0.446 | 0.62 | 0.325 |
| TIGA1 | 0.693 | 0.638 | 0.923 | −0.327 | 1.871 | 0.582 | 0.159 | 0.41 |
| TIM14 | 0.4665 | 0.6985 | −0.8755 | −0.1765 | 0.1855 | −0.0695 | −0.1625 | 0.1545 |
| TIMELES [symbol] | 0.9305 | −0.7105 | 0.0095 | 0.3065 | 0.9555 | 0.2335 | −0.1265 | −0.0085 |
| TIMM9 | −1.008 | −1.476 | −1.961 | −1.349 | −0.46 | −0.241 | −0.225 | −0.455 |
| TINP1 | 0.3275 | 0.4735 | 0.8705 | 0.0465 | 0.9955 | −0.4775 | 0.2485 | 1.3495 |
| [symbol] IP1208 | 0.243 | −0.072 | −0.695 | −0.595 | −0.031 | −0.381 | −0.04 | −0.222 |
| TK1 | 0.143 | −0.438 | −1.315 | 0.113 | 1.458 | −0.7 | −0.633 | 0.473 |
| TLE2 | −0.787 | 0.531 | −0.771 | −1.194 | 0.162 | 1.082 | −0.878 | 0.389 |
| TLR5 | 0.347 | −0.248 | 0.351 | 0.658 | 0.731 | 1.23 | 2.656 | 0.481 |
| TM4SF12 | 0.46 | 0.195 | 0.072 | −0.392 | 0.701 | 0.921 | −0.885 | 0.06 |
| TM4SF13 | −0.565 | 0.817 | 0.021 | 0.228 | −0.471 | 0.654 | 0.175 | 0.056 |
| TMEM14A | 0.1476 | 0.0085 | −0.2495 | −0.5575 | 0.2985 | 0.2305 | −0.1045 | −0.0515 |
| TMEM25 | −0.2335 | −0.6895 | −0.1575 | −0.9525 | −1.2275 | 0.1725 | −0.1765 | −0.4245 |
| TMEM4 | −0.339 | 0.052 | 0.978 | 0.236 | −0.891 | −0.266 | −0.312 | −0.38 |
| TMPRSS5 | −0.121 | 0.4 | 0.48 | −0.906 | −1.998 | −2.486 | 0.355 | −0.34 |
| TMSB10 | −2.183 | 0.588 | −0.227 | 0.888 | 2.09 | 0.048 | 1.867 | 1.44 |
| TMSNB | 0.9395 | −0.3715 | −0.2875 | −0.4815 | −0.5885 | −0.9205 | −0.8505 | 0.0175 |
| TNFRSF1 [symbol] | 1.147 | −0.444 | −0.348 | −0.344 | −1.13 | −0.585 | −0.919 | −0.103 |
| TNFRSF2 [symbol] | 1.2745 | −0.4775 | 0.4025 | 1.7755 | −0.4765 | −0.6765 | −0.3015 | −0.4615 |
| TNFRSF2 [symbol] | −0.5445 | −0.5715 | 0.2205 | −0.8985 | −1.6455 | −1.0445 | −0.5945 | 0.6255 |
| TNFSF4 | 0.9655 | −0.7045 | 0.5195 | −0.2445 | −0.5625 | −0.2185 | −1.5125 | 0.8105 |
| TNFSF7 | 0.5685 | 1.5635 | −0.8005 | −0.3705 | 0.2195 | −0.4835 | 2.8405 | −0.4715 |
| TNIK | 0.2225 | 0.0975 | −0.8275 | 0.0405 | −0.6215 | −0.9225 | −0.7575 | −0.3205 |
| TNNC2 | −0.62 | 0.692 | −0.615 | −0.318 | 4.472 | −0.741 | 0.033 | −0.03 |
| TNNI2 | 0.292 | 1.002 | 1.801 | 0.496 | −0.439 | 0.981 | 0.536 | 1.758 |
| TNNI3 | 0.1855 | −0.2795 | 0.1885 | 0.2825 | 2.8195 | 1.8355 | 0.8065 | 0.4315 |
| TOB1 | 0.3035 | −0.7595 | 0.0185 | −0.1325 | 0.1255 | −0.1225 | −0.8685 | 0.2815 |
| TOM1L1 | −0.2155 | 0.2685 | −1.0665 | −0.6085 | −1.2015 | −0.9415 | −1.2205 | 0.7145 |
| TOMM7 | 0.9185 | 0.0095 | 0.6775 | −0.6145 | 3.6205 | 1.0125 | 0.2885 | 1.0395 |
| TOP1MT | −0.222 | −0.257 | 0.121 | 0.075 | 0.744 | 0.506 | 0.455 | −0.004 |
| TOP2A | 0.2485 | 0.3895 | 0.4785 | 0.0755 | 0.5255 | 0.2295 | −0.1945 | 0.9425 |
| TOP2B | 0.647 | 0.811 | 0.434 | 0.27 | 1.79 | 1.11 | 0.658 | 0.888 |
| TOPBP1 | −0.8165 | 1.0185 | 0.7445 | −0.7215 | 0.3035 | 0.3595 | −1.1135 | 0.6955 |
| TOPK | −0.3375 | 0.4105 | −1.1575 | −0.1545 | 0.0615 | 0.5725 | −1.1735 | −1.2795 |
| TP53AP1 | 0.5905 | 1.1155 | −0.0575 | −0.1845 | 0.5785 | −0.4045 | 0.1945 | −0.6395 |
| TP53BP2 | 0.2075 | −1.7315 | −0.3705 | −1.6915 | −2.0675 | 0.1285 | 0.4175 | −0.7345 |
| TP53INP1 | −0.6215 | 0.4365 | 0.3085 | −0.0535 | −0.3835 | −0.4265 | 0.4505 | 0.1385 |
| TPD52L1 | 0.483 | 0.046 | −0.085 | 1.315 | 1.822 | 0.465 | 0.322 | 0.558 |
| TPRT | 1.0525 | 2.0475 | 1.2585 | −0.7725 | −0.4845 | 0.9825 | 0.9915 | 0.1005 |
| TRA@ | −0.183 | 0.248 | 0.631 | −0.502 | 0.137 | 0.056 | −0.151 | −0.648 |
| TRAF4 | 1.011 | 0.225 | −0.329 | 4.119 | 0.916 | 4.45 | 1.756 | 0.634 |
| TRAPPC4 | 0.988 | −0.025 | −0.376 | −0.423 | −0.401 | −0.110 | 0.084 | −0.317 |
| TRH | −1.3655 | −2.0455 | −0.9225 | −2.0445 | −1.9335 | −1.5555 | −0.0325 | −0.4155 |
| TRIM17 | −0.6295 | −1.4975 | −0.2915 | 0.6125 | −0.0415 | −0.2355 | −0.8005 | −0.5845 |
| TRIM2 | 1.1555 | −0.2425 | 1.9465 | −0.7285 | 1.4015 | 0.5365 | −0.2455 | 0.0995 |
| TRIM6 | 0.789 | 0.391 | −1.774 | −0.406 | 0.179 | −1.098 | 0.884 | −1.166 |
| TRIP3 | 0.3265 | −0.2205 | −0.2585 | 0.6015 | 0.5075 | 0.6775 | 0.7125 | 0.1085 |
| TRPS1 | −0.1415 | −0.2625 | −0.4155 | −0.1925 | −0.3275 | 0.4355 | 0.2335 | −0.5285 |
| TSG101 | −0.1995 | −0.0055 | 1.5015 | −0.1485 | 0.3115 | 0.2035 | −0.7115 | 0.0055 |
| TTC10 | −0.4235 | 0.3905 | 0.3415 | 0.0095 | 1.4495 | −0.8435 | 0.0915 | 1.3225 |
| TTC12 | −0.14 | −0.375 | −0.176 | −0.514 | −0.574 | −0.078 | −0.154 | −0.194 |
| TTK | −1.3285 | 0.3185 | −0.4076 | −0.2245 | −0.3425 | −0.5435 | −0.4175 | 0.5815 |
| TTLL4 | −0.433 | −0.155 | 1.864 | 0.452 | −0.907 | −0.007 | 0.896 | −0.2 |
| TTYH1 | 0.3625 | 0.0385 | −0.4495 | −0.4155 | −0.0925 | −0.5185 | −0.0935 | −0.5585 |
| TUBB3 | −0.0215 | −0.4725 | 0.4735 | −0.1985 | 0.1655 | 0.2985 | 0.2985 | 0.3055 |
| TULP3 | 0.405 | 0.939 | 0.976 | 0.075 | 1.525 | 0.845 | 1.355 | 1.113 |
| TXN | 1.204 | 0.674 | −0.375 | −0.312 | −0.527 | −0.234 | −0.492 | 0.811 |
| TYMS | −1.3815 | −2.4015 | −1.5085 | −1.0265 | −1.1425 | −1.1095 | −1.8805 | −1.3205 |
| U5-116KD | 0.8135 | 0.2815 | −0.3365 | −0.1285 | −0.0125 | −0.4325 | 0.0705 | 0.4975 |
| UBE2E3 | 0.333 | 0.467 | 0.051 | −0.237 | −0.817 | −0.228 | −0.317 | 0.372 |
| UBL5 | 0.3175 | −0.3155 | −0.6705 | −0.1845 | −1.8735 | −0.8825 | −0.2805 | −0.8735 |
| UBN1 | −0.72 | 0.308 | 0.093 | 1.067 | −0.25 | −0.072 | −0.818 | 0.339 |
| UCHL1 | 2.02 | 0.787 | −0.488 | −0.096 | 0.637 | 2.033 | 1.591 | 1.003 |
| UCHL3 | −0.411 | 0.304 | −0.271 | 0.477 | 0.438 | 0.15 | −0.492 | 0.704 |
| UGCG | −1.0045 | 0.3505 | −0.2425 | −0.1135 | 1.8785 | 0.5865 | −0.3835 | −0.1735 |
| UGCGL1 | 0.0106 | −0.1005 | −0.2975 | 0.3685 | 0.2375 | −0.4985 | −0.5795 | −0.2745 |
| UGT2B7 | 0.0785 | 0.4945 | −0.2195 | 0.4025 | −0.5535 | 0.0485 | −0.0835 | 0.1325 |
| UNC13D | −0.015 | −1.212 | −0.805 | −0.698 | −0.337 | −0.112 | 0.616 | −0.711 |
| VAMP8 | 0.314 | −0.874 | −0.143 | 0.441 | 0.528 | −0.023 | 0.21 | 0.001 |
| VAPA | −0.7305 | 0.8325 | 1.0355 | −1.5525 | −0.1025 | 0.3935 | 0.5755 | −1.0305 |
| VAV1 | 0.025 | 5.277 | −0.684 | −0.439 | 3.522 | −0.254 | 1.339 | 0.434 |
| VAV3 | −0.312 | −0.034 | 1.703 | 1.373 | −0.279 | 0.583 | 0.628 | 0.002 |
| VCX3 | 0.48 | 0.185 | −0.24 | 0.117 | −0.327 | −0.139 | 1.062 | −0.074 |
| VDAC2 | −0.2715 | −0.2085 | 0.0245 | −0.0115 | 0.0135 | −0.2015 | −0.1115 | −0.1035 |
| VEST1 | 0.173 | 0.062 | 0.009 | 0.026 | −0.07 | 0.203 | 0.366 | 0.054 |
| VGLL1 | −0.1355 | −0.4385 | −0.5085 | −0.4905 | −0.0035 | −0.2845 | −0.7545 | 0.3665 |
| VMD2L2 | −0.745 | 0.729 | −0.633 | −0.128 | −0.688 | −0.565 | −0.08 | −0.221 |
| VSX1 | 0.879 | 0.173 | 0.064 | 0.368 | 0.748 | 0.452 | 0.417 | 0.024 |
| WARP | 0.069 | 0.193 | 0.307 | −0.709 | −0.221 | 0.404 | 0.311 | −0.428 |
| WDR26 | −0.0155 | −0.6605 | −0.3315 | −0.7815 | −0.0075 | −0.8165 | 0.0575 | 0.0555 |
| WDR6 | 1.011 | 1.214 | −0.27 | 3.974 | −0.812 | −0.165 | 0.504 | 1.634 |
| WHSC1L1 | −0.619 | 0.068 | −0.066 | −0.189 | −0.329 | 0.071 | −0.409 | 0.098 |
| WNT3 | −0.1895 | 1.0445 | 0.7265 | 0.2625 | −0.2435 | −0.1255 | 0.3765 | −0.1275 |
| WNT6 | 0.2315 | 0.5875 | −0.2305 | 0.8515 | −0.1445 | 0.4935 | 0.8545 | −0.0325 |
| WSB2 | −0.3245 | 0.5495 | 0.1215 | 0.4565 | 0.2535 | 0.1865 | 0.2755 | −0.3165 |
| WWP1 | 0.5055 | 0.4145 | 0.5095 | −0.2945 | 0.0135 | 0.5395 | 1.0865 | −0.1985 |
| WWP2 | 0.933 | 2.202 | 1.469 | 0.265 | −1.518 | 0.751 | −0.719 | 0.539 |
| XBP1 | 0.526 | 1.185 | −0.284 | −0.297 | 0.779 | 0.267 | −1.581 | 1.677 |
| XYLT2 | 0.3345 | −0.3595 | 0.3775 | 0.0055 | 0.0135 | −0.0805 | 0.5475 | 0.7155 |
| YBX2 | −0.8 | −0.377 | −0.127 | −0.264 | 0.549 | 0.491 | 0.97 | −0.675 |
| YIF1 | 0.4445 | 0.8375 | −0.1045 | 0.0465 | 0.4145 | 0.0595 | 0.4055 | 0.8475 |
| ZA20D3 | 1.1005 | −0.6005 | −0.7265 | −0.4455 | −0.4435 | −0.1335 | −0.9405 | −0.7945 |
| ZAP128 | −0.1455 | −0.5625 | −0.2115 | −0.3255 | 0.2195 | −0.1145 | −0.3715 | −0.0635 |
| ZBED3 | 0.734 | 0.943 | −0.388 | 0.098 | −0.473 | −0.488 | 0.188 | 0.383 |
| ZBTB4 | −0.455 | 2.089 | 0.367 | −0.257 | −0.172 | −0.122 | −0.141 | 0.724 |
| ZCWCC1 | 0.2365 | 0.1685 | −0.4965 | −0.4325 | 0.1495 | 0.5955 | 0.5245 | 0.4475 |
| ZCWCC2 | −0.59 | 0.572 | −0.976 | −0.877 | −1.382 | −0.888 | −0.437 | −0.006 |
| ZDHHC13 | 0.1795 | −1.6855 | −0.8985 | 0.2495 | −1.0195 | −0.5875 | −0.2795 | 0.1576 |
| ZDHHC14 | −0.2435 | 0.5165 | −0.5575 | −0.7075 | −0.1235 | 0.1405 | −0.2285 | 0.8625 |
| ZFHX4 | 1.1095 | 0.3955 | −1.5845 | −0.5525 | 0.3945 | 0.0705 | 0.5275 | −0.1625 |
| ZFYVE21 | 0.27 | 1.47 | −0.622 | 0.191 | 2.422 | 1.046 | 0.4 | 1.038 |
| ZNF133 | 0.13 | 0.327 | −0.501 | −0.283 | 0.511 | 0.201 | 0.767 | 0.427 |
| ZNF165 | 0.1695 | 0.2915 | −0.3055 | −0.0855 | −0.1625 | 0.0745 | −0.1755 | 0.4215 |
| ZNF24 | 0.001 | −0.805 | −0.092 | −0.283 | 0.171 | 0.604 | −0.418 | 0.388 |
| ZNF318 | 0.0105 | 0.4265 | −0.0865 | −0.2275 | 0.7065 | −0.1015 | −0.1125 | 0.1605 |
| ZNF54 | 1.0495 | −2.6705 | 0.9035 | −1.8985 | −1.7065 | −1.5575 | −0.8005 | −0.4955 |
| ZNF432 | −0.5525 | 0.6925 | 0.2385 | −1.0725 | 0.2845 | −0.6365 | −1.0185 | 0.2665 |
| ZNF482 | 2.438 | 0.307 | 0.538 | 0.652 | 0.82 | 2.063 | 1.327 | 1.218 |
| ZNF505 | −0.971 | 0.76 | −9.487 | −0.321 | 1.77 | 0.888 | −0.685 | −0.43 |
| ZNF552 | 2.1745 | 0.1525 | 0.6855 | 0.8355 | 0.1735 | 1.0735 | 0.6375 | 1.4385 |
| ZNF577 | −0.4835 | 0.3135 | −0.4095 | −0.3755 | −0.3025 | 0.2315 | −0.2505 | 0.3045 |
| ZNF587 | 0.2575 | −0.3075 | −0.3685 | −1.3795 | −0.1065 | −1.1935 | −0.2436 | 0.2055 |
| ZNF598 | 0.1995 | 1.3305 | 0.1385 | 0.0895 | 0.4125 | 0.5775 | −0.1045 | 1.1855 |
| ZNF599 | −0.837 | 0.151 | −0.292 | −0.494 | 0.026 | 0.635 | 0.142 | −0.009 |
| ZNF606 | −0.032 | −0.029 | 0.228 | −0.185 | 0.457 | 1.217 | 0.163 | −0.404 |
| ZNF614 | 0.001 | 0.133 | −0.434 | 0.114 | 0.105 | −0.054 | 0.035 | 0.597 |
| ZNF639 | | | | | | | | |
| ZNF7 | | | | | | | | |

| | Sample | | | | | |
|---|---|---|---|---|---|---|
| | BC00015 | BC00034 | PB328-LN | PB211 | BR01-024 [symbol] | PB475 |
| | Class | | | | | |
| Symbol | LUMINAL | LUMINAL | LUMINAL | LUMINAL | LUMINAL | LUMINAL |
| | 1.3895 | 1.4905 | 1.0375 | 3.0865 | 1.8925 | 1.5455 |
| | −0.521 | −0.253 | −0.671 | −0.093 | 0.045 | −0.731 |
| ABAT | 1.637 | 1.261 | 0.782 | −0.146 | −0.114 | 0.694 |
| ABC1 | 0.0885 | 0.3335 | 0.1225 | 0.2925 | −0.1855 | −0.5695 |
| ABCC11 | 2.1475 | 0.9445 | 0.6235 | 0.3145 | 0.4775 | −0.3185 |
| ABCC3 | −0.97 | −0.321 | −0.122 | −0.685 | 0.002 | 0.706 |
| ACAS2L | −0.3435 | 0.9485 | 0.9455 | 0.3365 | −0.7415 | −0.9105 |
| ACAT1 | −0.254 | 1.945 | −0.803 | −0.715 | −0.366 | 0.974 |
| ACAT2 | −0.1755 | 0.0795 | −0.8855 | −0.1765 | 0.6345 | 0.6335 |
| ACOX2 | 0.1205 | 0.3205 | 1.2585 | 0.7195 | −0.2085 | −0.4335 |
| ACDX3 | −0.8515 | −0.4835 | −1.2445 | 1.5085 | −0.0855 | 0.3725 |
| ACSL3 | −0.215 | 0.858 | −0.089 | 0.058 | 0.533 | 0.654 |
| ACTA1 | −0.2875 | −0.1035 | 0.0965 | −0.0025 | −0.1435 | −0.1785 |
| ACTB | 1.9665 | 1.6535 | 2.2265 | −0.2985 | −0.2755 | −0.6635 |
| ADAM33 | −1.157 | 0.464 | −0.823 | 2.817 | 1.627 | 0.323 |
| ADAMDE [symbol] | −0.3995 | 0.5085 | −0.5565 | −0.5335 | −0.8135 | −0.0355 |
| ADPN | 0.7035 | 0.6955 | −0.4485 | −0.6865 | 0.1645 | 0.0645 |
| ADPRHL1 | 1.6835 | 1.0425 | 4.3815 | 3.7245 | 0.3705 | 1.5755 |
| ADRA2C | 0.4775 | −0.3845 | −0.9775 | 0.8065 | 0.3095 | 0.8695 |
| AGR2 | −0.2505 | 1.2135 | −0.2335 | −0.1765 | −0.4245 | −0.2655 |
| AGT | −0.485 | −0.061 | −0.229 | 0.381 | 0.201 | 0.005 |
| AGTR1 | −1.166 | 0 | 0.125 | 0.009 | −0.687 | 0.472 |
| AHCYL1 | 0.388 | −0.643 | −0.275 | 0.101 | −0.615 | 0.411 |
| AHNAK | 0.899 | 1.078 | 1.112 | 0.054 | −0.092 | −0.826 |
| AHR | 0.4515 | 0.2685 | 0.5235 | 0.2135 | 0.2455 | 0.3635 |
| AIM1 | 0.2565 | 0.0876 | 0.2225 | 0.1895 | −0.1115 | −0.1385 |
| AK2 | 0.4755 | −0.8375 | −1.2045 | 0.0435 | 0.0465 | −0.1095 |
| AK3L1 | −0.692 | −0.596 | −0.428 | 0.324 | −0.334 | −0.804 |
| AK5 | −0.208 | 0.855 | 0.79 | 0.113 | 0.101 | 0.014 |
| AKAP1 | −0.9965 | 1.1955 | −0.0935 | 0.7185 | 0.4255 | 0.3075 |
| AKR1A1 | −0.168 | 0.386 | −0.287 | −0.171 | −0.038 | 0.114 |
| AKR7A3 | 0.9165 | −0.0855 | −0.2235 | −0.4275 | −0.1185 | −0.0755 |
| ALG5 | 0.2605 | 1.2245 | −0.2375 | 0.2845 | −1.0475 | −1.7525 |
| ALG8 | 0.2785 | −0.8695 | −0.4085 | −0.2425 | −0.5225 | −0.0265 |
| AMFR | −0.548 | 0.263 | 0.656 | 0.273 | 0.105 | −0.869 |
| AMPD2 | 2.414 | 1.122 | 1.352 | 0.648 | −0.394 | 0.483 |
| ANAPC11 | 0.221 | −0.164 | 0.297 | 0.481 | −0.878 | −0.051 |
| ANKRD22 | −0.237 | 0.156 | 1.017 | 0.13 | −0.386 | 0.533 |
| ANKRD27 | 0.584 | −0.02 | −0.189 | 0.492 | 0.242 | −0.09 |
| ANLN | 0.068 | −1.538 | −1.559 | 0.922 | −1.54 | −0.013 |
| ANP32A | −0.693 | 0.475 | −0.105 | 0.87 | 0.586 | 0.398 |
| ANXA3 | −0.0405 | 0.6105 | 2.7355 | 0.2405 | −1.0615 | −0.1855 |
| APBB2 | 3.4605 | −1.3505 | −1.3885 | −0.4365 | −0.6575 | 0.3135 |
| APOBEC3 | 0.999 | 0.535 | 1.292 | 0.596 | 0.675 | 0.715 |
| AQP5 | −0.085 | 0.106 | −0.798 | 2.171 | 1.827 | 1.72 |
| AR | 0.7505 | −0.1705 | 0.8725 | 0.5585 | −0.1045 | 0.1275 |
| AREG | 1.28 | 0.09 | 1.768 | −0.275 | 0.677 | 0.764 |
| ARF1 | −0.4225 | 0.3675 | 0.3535 | −0.0105 | 0.2725 | −0.4385 |
| ARHGDI [symbol] | 0.414 | 0.198 | 0.347 | 0.778 | −0.456 | −0.325 |
| ARL6IP | 0.5185 | −0.4295 | −0.0305 | 0.2835 | 0.2615 | 0.3965 |
| ARL6IP2 | 0.132 | 2.038 | 3.538 | 0.805 | 1.242 | 0.338 |
| ARMCX2 | 0.224 | 2.068 | 3.607 | 0.74 | 1.297 | 0.038 |
| ARNT2 | 0.226 | −0.014 | −1.197 | −0.039 | −0.627 | −0.302 |
| ARNT2 | 0.4445 | 0.0085 | −0.0605 | −0.6355 | 0.9575 | −0.0385 |
| ARP3BET [symbol] | 1.1085 | 0.2045 | 0.8445 | −0.0195 | 0.0625 | 0.2295 |
| ARPC1B | 3.07 | 0.032 | −0.311 | 0.843 | −0.232 | −0.143 |
| ARPC5L | 0.8555 | 0.3285 | 1.1985 | −0.0485 | 0.3765 | 0.2405 |
| AS89 | 0.038 | 0.13 | −0.302 | −0.101 | −0.121 | 0.067 |
| ASF1A | 0.3555 | 0.7885 | 1.0825 | 0.5225 | 0.0935 | 0.0315 |
| ASH2L | −0.458 | 0.455 | 1.728 | 0.209 | −0.181 | −0.47 |
| ASK | −0.493 | 0.929 | −0.554 | −0.228 | 0.178 | 0.421 |
| ASPM | −0.0855 | 0.4585 | 0.7245 | 1.3045 | 0.1395 | −0.3565 |
| ASS | −0.0375 | 0.2825 | 0.2905 | 0.2855 | 0.4515 | −0.2485 |
| ATAD2 | 1.1365 | −0.0525 | 0.0705 | −0.3255 | 0.2215 | −0.5925 |
| ATF5 | 0.855 | −0.274 | 0.234 | −0.195 | −0.259 | −0.292 |
| ATF7IP2 | −0.302 | −0.213 | 0.554 | 0.979 | −0.17 | −0.451 |
| ATP2B1 | −0.063 | 0.303 | 0.159 | 0.33 | −0.206 | 0.038 |
| ATP5G1 | 0.2355 | 0.2455 | 0.0425 | −0.2185 | 0.4795 | −0.3845 |
| ATP5O | 0.838 | −0.423 | −1.038 | −0.557 | −0.798 | −0.704 |
| ATP6AP1 | −0.428 | −0.212 | 0.342 | −0.635 | −0.187 | 1.443 |
| ATP6V0A4 | 0.87 | −0.347 | 0.803 | 0.385 | 0.033 | 0.168 |
| ATP6V1B [symbol] | 0.3385 | 1.4545 | 0.1645 | 0.5725 | 0.1115 | 0.3065 |
| ATP6V1G [symbol] | −0.441 | 0.409 | 0.486 | −0.221 | −0.48 | −0.252 |
| ATPIF1 | −0.8335 | −0.9785 | −0.7605 | −0.6535 | −0.0115 | −0.2055 |
| ATXN10 | 0.197 | −0.725 | −0.847 | 0.381 | 0.191 | −0.047 |
| B3GNT5 | 0.6225 | −0.1845 | −0.2635 | 0.5035 | 0.2095 | 0.0295 |
| BACE2 | 0.5825 | 0.2495 | −0.1305 | 0.2935 | −0.5215 | 0.2595 |
| BAG1 | 0.6215 | 0.7535 | 1.5855 | −1.0885 | 1.7255 | 0.7585 |
| BAG4 | 1.6725 | −0.5705 | −2.5985 | −1.6165 | −0.1655 | −0.0985 |
| BAI2 | 1.934 | −0.422 | 1.497 | 0.333 | 9.006 | −0.398 |
| BAMBI | 1.212 | 1.526 | 1.128 | 0.07 | 0.295 | −0.251 |
| BCAS1 | 2.555 | 2.258 | 2.678 | 1.503 | 0.543 | 0.989 |
| BCL2L14 | 0.0935 | −1.1325 | −0.4165 | −0.4105 | 2.7485 | −0.5105 |
| BCMP11 | 0.073 | −1.735 | −0.318 | −0.731 | 1.145 | −0.647 |
| BEX1 | 2.1765 | 2.5325 | 0.9175 | 0.0025 | 2.3085 | 1.6855 |
| BEX2 | −0.2595 | 0.1455 | −0.7785 | −0.4685 | −0.7005 | 0.1515 |
| BF | 0.324 | 0.399 | 0.138 | −0.21 | −0.497 | −0.194 |
| BFSP1 | 0.223 | 0.311 | 0.192 | 0.035 | 0.093 | 0.189 |
| BIK | −0.969 | 0.192 | 0.738 | −0.266 | 0.95 | −0.811 |
| BIRC2 | −0.2415 | 0.0575 | −0.0435 | −0.3485 | −0.0995 | −0.5665 |
| BIRC5 | 0.0905 | −1.0375 | −0.3755 | −0.6305 | −0.1885 | −0.5255 |
| Bit1 | 0.8855 | 0.9305 | 0.6885 | 0.6375 | 0.3775 | 1.5545 |
| BIVM | −0.179 | 0.231 | 0.71 | −0.052 | 0.254 | −0.435 |
| BLVRA | 1.603 | −0.642 | −1.342 | −1.288 | −0.340 | 0.001 |
| BM039 | −0.444 | −0.415 | −0.261 | −0.351 | 0.572 | 0.323 |
| BMP7 | −0.706 | 1.02 | 0.868 | 0.806 | −0.35 | −0.766 |
| BOC | 0.3385 | −0.1885 | −0.2765 | 0.4085 | −0.8645 | −0.0325 |
| BRCA1 | −0.6575 | −0.5885 | −0.6285 | −0.6975 | −0.7905 | 0.0785 |
| BRF2 | −0.21 | 0.313 | −0.097 | −0.773 | 0.57 | −0.101 |
| BRI3 | 0.644 | 1.028 | −0.178 | 0.451 | 0.197 | −0.031 |
| BRI3BP | 0.488 | 0.808 | 0.092 | 0.492 | −0.092 | −0.123 |
| BSPRY | −0.8845 | 0.6875 | 0.0045 | −1.4765 | −1.1345 | −0.9675 |
| BTF3 | 1.1715 | 1.1265 | 0.7125 | −0.0255 | 0.7195 | −0.2975 |
| BTG3 | 0.48 | 0.817 | 1.314 | −0.141 | 0.028 | −0.067 |
| BγN2A2 | 0.381 | 0.016 | 0.776 | 1.303 | 0.002 | 0.509 |
| BUB1 | 1.1485 | −0.2425 | −0.4715 | −0.2735 | 0.0185 | −0.7125 |
| BUB3 | −0.94 | −0.285 | −0.117 | −0.868 | −0.393 | −0.18 |
| BXDC1 | 0.404 | 0.55 | 1.535 | 0.681 | 0.006 | 0.078 |
| BZRP | 0.032 | 0.006 | 0.88 | −0.812 | −1.171 | 0.187 |
| BZW1 | −0.5285 | −0.8175 | −0.3235 | −0.1385 | −0.3055 | 0.0005 |
| C10orf116 | 0.1485 | 0.5525 | −0.1135 | −0.2605 | 0.3575 | −0.1995 |
| C10orf38 | 1.183 | −0.078 | 1.46 | −0.121 | 0.117 | −0.155 |
| C10orf4 | 0.2855 | −0.0875 | −0.1315 | 0.2045 | −0.3125 | −0.3425 |
| C10orf45 | −0.6905 | −0.6105 | 0.5115 | 1.2095 | 0.5595 | 1.6055 |
| C10orf7 | 0.1105 | −0.1145 | −0.2325 | 0.0925 | −0.5165 | 0.2085 |
| C10orf82 | −0.277 | −1.506 | −1.003 | −0.63 | 0.632 | −0.55 |
| C11orf24 | 0.2605 | −0.0515 | −0.8805 | −0.8705 | −0.2515 | −0.3825 |
| C12orf14 | 0.086 | 0.444 | 0.604 | −0.505 | −0.193 | 0.145 |
| C13orf21 | −0.416 | 0.099 | −0.28 | 0 | 0.04 | 0.162 |
| C13orf23 | 0.401 | 0.605 | 1.335 | −0.008 | −0.382 | −0.296 |
| C14orf94 | −0.5 | −0.46 | −0.794 | −1.016 | 0.12 | −0.12 |
| C15orf23 | −0.7185 | 0.1415 | 0.1395 | 0.4435 | −0.4965 | −0.5725 |
| C16orf45 | −0.495 | 0.886 | −0.015 | 0.307 | −0.05 | 0.764 |
| C17orf37 | 0.098 | 0.125 | −0.286 | 0.377 | 0.192 | −0.283 |
| C18orf1 | 0.703 | 0.008 | −0.522 | −0.082 | 0.611 | −0.019 |
| C19orf10 | −0.8265 | −0.8835 | −0.5035 | −0.2415 | 0.4825 | −0.4055 |
| C19orf32 | 1.2545 | 0.4645 | 0.9085 | 0.5745 | −0.4885 | −0.5915 |
| C19orf33 | 1.3 | 1.517 | 0.948 | 1.696 | 1.024 | −0.148 |
| C1orf24 | −0.108 | 0.514 | −1.132 | −0.787 | −0.578 | 1.222 |
| C1orf34 | −0.0025 | −0.2485 | 0.0775 | 0.2425 | 0.2905 | 0.0135 |
| C20orf103 | 0.5685 | 0.3355 | −0.4605 | 0.1835 | −0.3195 | −0.0995 |
| C20orf149 | 2.359 | 0.035 | −0.234 | −0.174 | 0.248 | 0.578 |
| C20orf23 | −0.859 | −0.103 | −0.832 | −0.411 | 0.198 | 0.087 |
| C20orf3 | 0.197 | −0.549 | −0.129 | −0.383 | 0.37 | −0.071 |
| C20orf35 | 0.304 | −0.299 | −0.109 | −0.22 | −0.208 | −0.68 |
| C20orf43 | 0.2025 | 0.4985 | −0.2675 | 0.1755 | 0.2275 | −0.1045 |
| C20orf8 | −0.638 | 0.443 | 1.218 | −0.312 | −0.311 | −0.365 |
| C21orf45 | 0.059 | 0.318 | 0.296 | −0.004 | 0.322 | 0.129 |
| C22orf18 | −0.167 | −0.234 | −0.313 | −0.017 | 0.18 | 0.221 |
| C2F | 0.848 | 0.706 | 0.263 | −0.875 | −0.168 | 1.42 |
| C2orf17 | −0.253 | 0.998 | 0.273 | −0.244 | −0.452 | 0.361 |
| C4A | 0.264 | 0.197 | −0.518 | −0.246 | −0.169 | −0.474 |
| C5orf13 | 0.043 | −0.717 | −0.409 | −0.18 | 0.287 | −0.622 |
| C6orf108 | 0.012 | 0.57 | 0.032 | −0.738 | −0.337 | −0.648 |
| C6orf166 | 1.785 | 0.797 | 0.362 | 1.574 | 1.182 | 0.661 |
| C6orf192 | 0.1345 | −0.1185 | 0.4015 | 0.5185 | 0.1815 | −0.2285 |
| C6orf211 | −0.139 | 0.94 | −0.069 | 2.73 | 0.532 | 0.634 |
| C6orf49 | 1.5575 | 0.8265 | 2.3515 | 0.5675 | −1.3175 | −0.2105 |
| C6orf85 | 0.1975 | −0.4955 | 0.1675 | −0.1865 | 0.3265 | 0.2385 |
| C7orf24 | −0.472 | 2.82 | −1.548 | 2.783 | 1.095 | 1.005 |
| C9orf10 | 0.691 | 0.026 | −0.142 | 0.395 | 0.001 | −0.439 |
| C9orf121 | −0.232 | 0.228 | 0.21 | −0.397 | 0.578 | 0.127 |
| C9orf58 | −0.2795 | 0.7975 | −0.4115 | −0.3255 | −0.0885 | −0.2965 |
| C9orf88 | 1.083 | 0.015 | −0.351 | 1.085 | 1.233 | −0.051 |
| C9orf9 | −1.686 | 0.756 | −0.15 | 0.199 | 0.257 | −1.184 |
| CA12 | −1.924 | 1.064 | −0.228 | 0.193 | 0.381 | −1.305 |
| CA8 | −0.007 | −0.224 | −0.267 | −0.163 | −0.068 | 0.15 |
| CA8 | 0.0515 | −0.1935 | −0.0195 | 0.0475 | 0.3115 | −0.1415 |
| CACNA1G | 3.346 | 0.668 | 0.078 | −0.218 | −0.38 | −0.402 |
| CALCA | 1.931 | −0.68 | −0.758 | −0.716 | 0.099 | 2.129 |
| CALML4 | 0.3195 | 0.7185 | −1.5285 | −0.8475 | 0.8205 | 0.2525 |
| CALML5 | −0.0405 | 0.8245 | 0.4035 | 0.8215 | 0.9145 | 0.0375 |
| CaMKIINa [symbol] | 0.209 | 0.271 | 0.629 | 0.078 | 0.272 | −0.202 |
| CAP2 | 1.624 | 1.708 | −0.228 | −0.9 | −0.068 | −1.084 |
| CAP350 | 0.4505 | −0.4196 | −0.4125 | 1.6995 | 0.4455 | 0.7165 |
| CAPN13 | −0.468 | 0.333 | −0.004 | −0.529 | −0.474 | −0.168 |
| CAPN9 | 0.4135 | −0.2185 | 0.6055 | −0.1155 | −0.1965 | −0.0285 |
| CARD14 | 1.0225 | 1.5885 | 1.5725 | −0.0455 | 0.4445 | 0.1885 |
| CART | −0.0115 | 0.4265 | 0.0925 | 0.4805 | 0.3035 | −0.0005 |
| CASP1 | −0.068 | 1.051 | 0.895 | −0.002 | −0.206 | −0.057 |
| CASP3 | −0.263 | −0.243 | −0.266 | −0.215 | −0.166 | −0.041 |
| CASP7 | −0.4055 | −0.1655 | −0.4625 | −0.1475 | 0.2245 | 0.0645 |
| C9LN1 | 0.461 | −0.642 | 1.805 | 1.245 | 0.268 | −0.262 |
| CBR1 | −0.7395 | 1.2155 | −0.1465 | −0.2345 | 0.1295 | 0.6225 |
| CBR3 | −0.416 | −0.35 | −0.213 | −0.265 | −0.115 | −0.259 |
| CBR4 | 0.0025 | −1.6405 | 1.1905 | 1.9135 | 0.2055 | −0.1765 |
| CCL24 | −0.266 | 0.414 | 0.866 | 0.024 | 0.152 | 0.06 |
| CCL29 | −0.159 | −1.073 | −2.678 | −0.253 | 0.908 | 0.681 |
| CCNB2 | −0.1345 | −0.4175 | 0.1065 | −0.2265 | −0.3165 | −0.0505 |
| CCND1 | 0.534 | 0.828 | −0.42 | 1.034 | −0.407 | 0.27 |
| CCNE1 | 0.0845 | 0.7855 | 0.8185 | 0.1225 | −0.0335 | 0.1505 |
| CCNG2 | 1.496 | 1.331 | 1.495 | −0.432 | 1.017 | −0.041 |
| CCNH | 1.7995 | 0.9455 | 3.1535 | 0.1205 | 1.9445 | 0.1635 |
| CD38 | −0.1465 | 0.1716 | 0.0805 | −0.4445 | 0.6305 | −0.3795 |
| C030 | 0.8215 | −0.3085 | 0.3965 | 0.4995 | −0.2535 | 0.3545 |
| CD4 | −0.3585 | −1.2965 | −1.2585 | −0.3015 | −0.1225 | −0.4365 |
| CD58 | −0.259 | 2.486 | 1.828 | 0.206 | 0.222 | 0.184 |
| CD50 | −0.6225 | 0.9215 | 0.9125 | 0.9225 | −0.2365 | −0.0405 |
| CDC2 | −0.0525 | 0.1985 | 1.2545 | 0.2395 | 0.3275 | −0.5235 |
| CDC6 | −0.0575 | 0.2495 | 0.5595 | −0.0005 | 0.0935 | −0.4575 |
| CDCA1 | −0.354 | 0.427 | 1.618 | 1.05 | −0.523 | 0.054 |
| CDCA5 | −3.351 | 1.535 | −1.127 | −1.904 | 0.008 | −0.259 |
| CDCA7 | 0.2535 | −0.8545 | −1.0285 | 0.4375 | −0.1905 | −0.4135 |
| CDH1 | −0.534 | 0.417 | 0.134 | 0.097 | 0.072 | −0.487 |
| CDH3 | −0.4175 | −0.8925 | 0.9285 | −0.3505 | −0.8565 | −0.2265 |
| CDK2 | −0.1125 | 1.1395 | 1.0635 | −0.3825 | 0.1765 | −0.7225 |
| CDKN2A | 0.0005 | 0.6735 | 0.6505 | 0.0645 | 0.0805 | −0.1805 |
| CDKN2D | −0.825 | 1.048 | 0.432 | −0.388 | −0.276 | −0.478 |
| CDKN3 | 0.005 | 0.154 | 0.679 | 0.117 | −0.19 | −0.595 |
| CDT1 | 0.6945 | 1.0975 | 1.2545 | 0.5425 | −0.1255 | 0.5045 |
| CE9PG | −0.446 | 0.211 | 0.457 | −0.593 | 0.36 | −0.299 |
| CELSR1 | −0.318 | 0.177 | 0.674 | 0.054 | −0.141 | −0.189 |
| CENPA | −0.008 | 0.425 | 1.024 | 0.263 | 0.29 | 0.015 |
| CENPE | −1.32 | −0.006 | −1.035 | −0.178 | 0.318 | 0.342 |
| CENPF | −0.3195 | 0.2625 | −0.2685 | −0.2705 | −0.5385 | −0.0685 |
| CENTG3 | 0.5375 | 0.1025 | 0.0585 | 0.0865 | −0.1245 | −0.4455 |
| CES2 | −0.445 | −0.427 | −0.179 | 0.093 | 0.811 | 0.33 |
| CETN3 | 0.1066 | −0.5365 | −0.5885 | 0.3585 | −0.1795 | −0.5125 |
| CGA | −0.2005 | −0.4365 | 0.5815 | −0.4045 | −0.3055 | −0.8345 |
| CGI-12 | −0.2755 | 1.1875 | −0.7425 | 1.2765 | 0.0165 | −0.0315 |
| CGI-49 | 1.179 | 0.824 | −0.493 | 0.909 | −0.071 | −0.332 |
| CGI-4 [symbol] | 1.465 | −0.208 | 0.651 | 0.443 | 0.856 | −1.014 |
| CGN | 0.077 | 1.019 | 0.603 | 0.243 | 0.007 | −0.059 |
| CGNL1 | −0.3315 | −0.1745 | −0.5655 | −1.0065 | −1.0525 | −0.1525 |
| CHAF1B | 0.149 | 0.777 | 0.638 | 0.726 | −0.068 | 0 |
| CHC1 | −0.552 | −0.006 | −0.056 | −0.036 | −0.304 | −0.286 |
| CHCHD2 | −0.013 | −0.682 | −0.625 | −0.299 | −0.606 | −0.514 |
| CHCH05 | 1.592 | −1.12 | −1.005 | −0.826 | −0.952 | −0.822 |
| CHI3L1 | 0.227 | −0.189 | −1.087 | −0.126 | 0.077 | 0.083 |
| CHI3L2 | −0.152 | −0.175 | 0.004 | −0.088 | −0.296 | −0.3 |
| CHKA | −0.09 | −0.351 | −0.34 | −0.046 | −0.395 | −0.265 |
| CHCDL | −0.4035 | −0.2755 | −0.0345 | −0.2195 | −0.2285 | 0.1605 |
| CHRDL2 | 0.553 | −0.126 | 0.118 | 0.245 | −0.387 | 0.419 |
| CHST2 | 0.7945 | 0.0655 | −0.0055 | 0.3885 | −0.1405 | −0.1415 |
| CHURC1 | −0.048 | 0.542 | −0.718 | 0.122 | 0.7 | 0.132 |
| CIR | −0.3005 | 0.2805 | 0.2095 | −0.6305 | −0.3025 | −0.0385 |
| CIRBP | 0.1125 | 1.3905 | 0.2605 | 1.2225 | 0.1625 | −0.0395 |
| CKLF | −0.4445 | −0.1505 | −0.2375 | 0.5925 | 0.3775 | 0.3095 |
| CKLFSF6 | 0.7835 | −1.6445 | 1.0355 | 0.5875 | −0.5755 | −1.0555 |
| CKLFSF7 | 0.116 | 0.004 | 0.607 | 0.24 | −0.484 | −0.218 |
| CKMT1 | 0.42 | 1.003 | 1.228 | 0.628 | 0.972 | −0.182 |
| CKS1B | −0.334 | 0.674 | −0.005 | −0.36 | −0.296 | −0.572 |
| CKS2 | 0.2385 | 0.5015 | −0.3055 | 0.4595 | −0.9455 | 0.0785 |
| CL640 | 1.0595 | −0.4135 | −0.4335 | −0.2135 | −0.3735 | 0.3105 |
| CLDN23 | 0.9885 | −0.7455 | −1.3285 | −0.1195 | −0.4965 | −0.8125 |
| CLDN3 | −0.5655 | 0.4625 | −0.7355 | −0.1715 | −0.6115 | −0.6075 |
| CLDN4 | 1.785 | 0.403 | 2.015 | 2.186 | 1.047 | 2.325 |
| CLDN8 | −0.4045 | −1.0615 | −0.1605 | −0.3345 | 0.7945 | −0.6365 |
| CLGN | 0.2025 | −0.1025 | −0.3245 | −0.5185 | −0.1025 | 0.0015 |
| CLIC6 | −0.056 | −0.018 | 0.769 | −0.434 | 0.106 | −0.352 |
| CLNS1A | 1.7315 | −1.0225 | −1.2585 | −1.3285 | −0.4965 | −0.9676 |
| CLTC | 1.201 | −0.197 | 0.401 | 0.873 | 0.166 | −0.201 |
| CLU | 0.098 | −0.727 | −0.42 | −0.15 | −0.989 | −0.688 |
| CMAS | 0.2885 | 0.4455 | −0.2905 | 0.5895 | −0.0605 | −0.2105 |
| CML65 | −0.175 | 0.716 | 0.42 | 0.371 | 0.423 | 0.178 |
| CNIH | 0.523 | 0.091 | −0.017 | 0.23 | 0.304 | 0.027 |
| CNOT10 | 0.6785 | 0.4365 | 0.3585 | 0.7495 | −0.4435 | 0.0375 |
| CNOT2 | 0.01 | 0.532 | −0.494 | 0.377 | −0.596 | −0.163 |
| CNOT7 | 0.176 | −1.288 | 0.769 | −2.085 | 2.734 | 3.072 |
| CNP | −0.4925 | 0.2615 | −0.2445 | 1.0935 | −0.2485 | −0.3465 |
| CNTNAP2 | −0.004 | −0.134 | −0.287 | 0.021 | −0.011 | 0.009 |
| COASY | 0.6585 | −0.6365 | −1.8005 | 1.1185 | −0.9255 | −0.6755 |
| COL11A2 | 0.876 | 1.233 | 0.648 | 0.905 | 0.796 | 1.017 |
| COL27A1 | −0.265 | −1.352 | 0.024 | 0.337 | −0.431 | 0.291 |
| COL4A5 | 0.0025 | −0.5805 | −1.4475 | −1.1416 | −0.0025 | −0.4655 |
| COL8A1 | 0.407 | 0.369 | 0.704 | 0.559 | 0.365 | 0.288 |
| COL8A3 | 0.578 | −0.052 | −0.645 | −0.123 | 0.364 | −0.004 |
| COMMD1 [symbol] | −0.1025 | −0.0015 | 0.1045 | 0.0325 | −0.1305 | −0.1115 |
| COMMD5 | −0.8565 | −0.2255 | 0.0176 | −0.1705 | 0.0055 | 0.2995 |
| COPS7B | −0.483 | 0.444 | −0.229 | −0.482 | 0.145 | 0.019 |
| CORO6 | 0.3685 | −0.4195 | 0.8455 | 0.1845 | 0.2885 | −0.1995 |
| CORT | 0.69 | 0.042 | 0.912 | 0.171 | −0.093 | 0.071 |
| COX17 | 0.796 | −0.124 | 0.885 | 0.107 | −0.054 | 0.1 |
| COX5A | 0.657 | 0.53 | 0.92 | 0.348 | −0.444 | −0.246 |
| COX6A | 0.1005 | −0.4465 | 1.3765 | 0.0785 | 1.5735 | −1.1725 |
| COX8A1 | 0.2275 | 0.1905 | 1.3195 | 0.4635 | −0.1295 | −0.6705 |
| COX8C | 4.1075 | −1.2545 | −0.2505 | −0.1585 | −0.8215 | −0.6085 |
| COX7A2 | −0.357 | −0.281 | −0.989 | −0.239 | −0.59 | −0.375 |
| CP | −0.3495 | 0.5685 | 1.0125 | 0.3135 | 1.3135 | −1.0385 |
| CPAMD0 | 0.377 | 0.889 | 0.476 | 0.723 | 0.26 | −0.33 |
| CPB1 | 0.8355 | 0.1905 | −0.1555 | 0.4045 | 0.1155 | −0.6275 |
| CPEB3 | 0.319 | 0.226 | −0.074 | 0.192 | 0.359 | −0.15 |
| CPEB4 | −0.602 | 1.257 | −0.328 | −0.544 | −0.413 | 0.466 |
| CPSF3 | 1.1775 | 0.3405 | 0.9145 | 1.4065 | 0.3925 | 0.7915 |
| CRABP1 | 0.519 | 0.557 | 0.101 | 0.108 | 1.192 | 0.126 |
| CREB3L4 | 0.724 | −0.578 | −0.046 | 0.499 | −0.275 | −0.166 |
| CRIP1 | −0.187 | 0.276 | −0.138 | −0.311 | −0.097 | −0.003 |
| CRYBB2 | 0.559 | 0.893 | −0.659 | 1.491 | 0.236 | −0.153 |
| CRYL1 | −1.447 | −0.308 | −0.803 | −0.744 | −0.114 | −0.142 |
| CRYZ | −0.0745 | 0.2075 | 0.2815 | −0.1255 | −0.0515 | −0.1215 |
| CSDA | 0.0875 | 0.2815 | 0.1635 | −0.2105 | 0.9785 | −0.0235 |
| CSE1L | 0.7035 | −0.7335 | −0.5135 | 0.6575 | 0.2295 | −0.1435 |
| CSK | −0.8785 | −0.8555 | −0.1195 | −0.3095 | −0.7955 | 0.0645 |
| CSRP1 | −0.0285 | −1.3905 | −1.5085 | 0.0225 | −0.3685 | 0.4485 |
| CSRP2 | −0.685 | 0.039 | −0.815 | 0.158 | −0.216 | 0.262 |
| CST5 | −0.7525 | 0.2245 | −0.7625 | 0.0205 | 1.2185 | 0.3965 |
| CSTB | −0.892 | 0.393 | −0.288 | 0.697 | 0.115 | 0.015 |
| CTAG1B | −0.0035 | 0.2265 | 0.0035 | 0.2445 | −0.2405 | 0.3215 |
| CTBP2 | 0.151 | −0.059 | 0.809 | 0.245 | −0.287 | −0.151 |
| CTNNA1 | −0.825 | 0.585 | −0.199 | −0.333 | 0.013 | 0.315 |
| CTNNAL1 | −0.0355 | −0.0545 | 0.4815 | −0.6945 | −0.2685 | −0.1015 |
| CTNND2 | 0.747 | 0.776 | −0.122 | 0.769 | −1.4 | −0.804 |
| CTPS | 1.832 | 0.442 | 1.562 | −0.307 | 0.3 | −0.453 |
| CTSL2 | 0.0555 | −0.3145 | −1.2285 | −0.3555 | −0.4335 | 0.6325 |
| CTSS | −1.34 | −2.09 | −1.232 | 0.155 | −0.7 | −1.04 |
| CTTN | 1.7595 | 1.4115 | 2.6765 | 0.2085 | 1.6025 | −0.3725 |
| CX3CL1 | 1.1785 | −0.1715 | 0.3785 | −0.0715 | 0.0795 | 0.1675 |
| CXCL9 | 1.6895 | 2.3616 | 1.4776 | −0.4425 | 1.1615 | −0.0485 |
| CXorf39 | 0.696 | 1.588 | −0.214 | 0.914 | 0.295 | 0.682 |
| CXXC4 | 0.312 | −0.444 | −0.023 | 0.542 | 0.001 | −0.524 |
| CYB5 | 0.3515 | −0.2985 | −0.1385 | −0.2125 | −0.1695 | −0.1965 |
| CYB561 | −0.623 | −0.176 | −1.572 | −0.313 | 0.764 | −0.475 |
| CYC1 | −0.301 | −0.161 | −0.063 | 0.015 | 0.01 | 0.096 |
| CYHR1 | 1.3145 | 0.1475 | −0.3975 | −0.5305 | 0.7145 | 0.7295 |
| CYP2A13 | −0.95 | 0.264 | 0.142 | −0.957 | −0.479 | −0.607 |
| CYP4X1 | 1.1805 | 1.6995 | 0.1465 | 1.2465 | 0.0105 | −0.0475 |
| D15Wsu7 [symbol] | 0.4225 | 1.4005 | 0.4575 | 0.2995 | 0.1415 | −0.3645 |
| DACH1 | 1.473 | 2.035 | 0.349 | 1.659 | −0.427 | −0.202 |
| DACH1 | 0.327 | −0.288 | 0.701 | 0.436 | 0.073 | 0.049 |
| DACH1 | 1.377 | −0.669 | 1.424 | 0.408 | 0.507 | −0.172 |
| DAP3 | 0.6235 | 0.3495 | 0.9245 | 0.6995 | −0.0295 | −0.0235 |
| DAPP1 | 0.632 | 0.976 | 1.432 | 0.266 | −0.152 | −0.264 |
| DAZAP2 | −0.904 | −1.282 | −0.607 | −0.235 | −0.37 | 0.127 |
| DBI | 0.0515 | 0.4435 | 0.2835 | −0.2495 | −0.2935 | −0.4485 |
| D9N1 | −0.23 | 0.711 | −0.043 | −0.499 | 0.346 | 0.061 |
| DC13 | −0.2535 | 0.2405 | −0.1135 | −0.3835 | −0.4725 | 0.2425 |
| DCPS | 0.6805 | 0.8345 | −0.3455 | −0.9675 | 0.0275 | −0.2035 |
| DDHD2 | 0.1455 | 0.2745 | 0.4875 | 0.1375 | 0.2235 | −0.7145 |
| D0X17 | −0.3495 | 0.1955 | −0.0395 | −0.0895 | 0.5185 | −0.1935 |
| DDX39 | 2.016 | 1.26 | 0.476 | −0.069 | −0.209 | −0.074 |
| DDX48 | 0.8255 | 0.8015 | 0.9595 | 0.5345 | 1.3265 | 0.3825 |
| DDX58 | 0.6025 | 0.0725 | 1.1285 | 0.1845 | −0.5905 | −0.1885 |
| DEF6 | −0.55 | −0.182 | −1.019 | 0.001 | −0.258 | −0.788 |
| DEK | 0.365 | 0.797 | 0.585 | 0.886 | −0.22 | −0.104 |
| DHCR7 | −0.451 | 1.083 | −0.732 | −0.223 | 0.012 | 0.449 |
| DHRS7 | −1.9915 | −0.9935 | 0.8075 | −0.3875 | −0.1975 | 0.6485 |
| DHX57 | −0.5025 | −2.1475 | 2.0275 | 0.2225 | −1.1275 | −0.5085 |
| DIRC1 | −0.91 | 0.194 | −1.321 | −1.117 | −0.041 | 0.071 |
| dJ222E13 | −0.342 | 0.018 | −0.08 | 0.441 | 0.096 | −0.516 |
| DKFZP434 | 0.077 | −0.568 | −0.287 | −0.101 | −0.638 | −0.843 |
| DKFZp434 | −0.5335 | 1.2195 | −0.0235 | 0.2195 | 0.2145 | −0.2965 |
| DKFZP434 | −0.66 | 0.672 | 0.177 | 0.315 | −0.223 | 0.415 |
| DKFZp434 | 0.3555 | 0.2745 | 0.4565 | 0.4135 | 0.2385 | 0.0815 |
| DKFZP564 | 0.5435 | −0.3925 | −0.2495 | −0.2265 | 0.0455 | −0.1145 |
| DKFZp564 | 0.75 | −0.514 | −0.849 | −0.244 | −0.698 | −0.542 |
| DKFZP564 | −0.1705 | 0.1505 | 0.1945 | 0.2375 | −0.2405 | −0.2595 |
| DKFZP564 | 1.336 | −0.305 | −0.085 | −0.05 | −0.187 | 0.18 |
| DKFZP564 | 0.046 | 1.052 | −0.589 | 1.924 | 0.435 | 1.17 |
| DKFZp586 | −0.7265 | 0.0235 | −0.5165 | 0.1285 | 0.8055 | 0.7985 |
| DKFZp761 | −0.211 | −0.404 | 0.154 | −0.211 | −0.06 | −0.133 |
| DLK1 | 0.964 | 0.037 | 0.635 | −1.06 | 0.622 | 0.27 |
| DLX5 | 0.6825 | 1.2355 | 1.8685 | −0.1355 | 0.6555 | 1.1515 |
| DNAJA4 | −0.6915 | 2.9705 | 2.1105 | 1.2875 | 1.2035 | −1.5335 |
| DNAJC1 | 0.094 | 0.071 | 0.047 | −0.602 | 0.155 | −0.351 |
| DNAJC12 | −3.272 | −2.075 | −3.227 | −1.291 | 1.357 | −0.629 |
| DNAJD1 | 0.4 | 1.319 | 0.847 | 0.273 | 0.442 | −0.237 |
| DNALI1 | 0.341 | 0.732 | 0.246 | 0.717 | −0.028 | −0.072 |
| DNMT1 | −0.3405 | −1.5245 | −1.2665 | 0.2815 | −1.3645 | −0.6235 |
| DOX1 | 0.4405 | 0.1785 | −0.0675 | 0.1525 | −0.5775 | −0.4595 |
| DSCR1 | −0.1515 | −0.6665 | −0.5305 | −0.5885 | 0.2505 | −0.1715 |
| DSCR1L2 | 0.0645 | 0.7275 | 0.6295 | 0.3465 | 0.0975 | −0.0265 |
| DSP | −0.2035 | −0.3615 | 0.3085 | −0.4555 | −0.5105 | −0.2915 |
| DTYMK | −0.2695 | −0.7895 | −0.6635 | −0.4475 | −0.4345 | −0.1165 |
| DUSP23 | −0.0755 | −1.4105 | −0.7175 | 0.2545 | 0.1915 | 0.1145 |
| DUSP4 | −1.0725 | 0.0515 | −1.2265 | −0.6225 | 0.3045 | 0.2505 |
| DUSP5 | 0.057 | −0.181 | −0.025 | −0.255 | 0.496 | −0.17 |
| DVL1 | −0.389 | −0.638 | −0.216 | 0.205 | −0.636 | −0.111 |
| DXYS155 [symbol] | −0.102 | −0.097 | −0.533 | −0.19 | −0.311 | −0.495 |
| DYRK4 | 1.8945 | 0.8045 | 2.0195 | 0.9905 | 0.7525 | −0.5285 |
| o(y)2 | −0.27 | −0.112 | 0.258 | 0.354 | −0.614 | −0.169 |
| EAF2 | −1.0385 | 0.3775 | −0.3005 | 0.8325 | −0.6135 | 0.1345 |
| EAP30 | −0.2325 | −3.9215 | 0.2895 | −0.0355 | −1.4755 | −0.3415 |
| ECHDC1 | −0.5915 | 0.8665 | 0.0755 | −0.2955 | 0.6275 | 1.3195 |
| ECHDC3 | −1.3215 | 1.1305 | −0.0415 | 0.0855 | −0.3065 | 0.0335 |
| EEF1A2 | −0.28 | −0.017 | −0.669 | −0.200 | 0.163 | 0.764 |
| EFHC1 | −0.3095 | 0.1165 | 0.0005 | 0.4135 | −1.6215 | −1.2325 |
| EFNA4 | −0.2695 | −0.2905 | −0.1205 | −0.2905 | −0.4865 | 0.0005 |
| EFS | −0.5265 | 0.0385 | 0.1255 | 0.2045 | −0.4005 | −0.0425 |
| EIF2C2 | 0.0635 | −0.4085 | −0.5115 | −0.3335 | −0.7235 | −0.0695 |
| EIF2C4 | −0.711 | 0.097 | −0.415 | −0.121 | 0.051 | −0.169 |
| EIF4EBP1 | −0.9175 | 0.1515 | −0.3905 | −0.8775 | 0.5276 | 0.3325 |
| EIF4G1 | 0.393 | −0.819 | −0.156 | 0.466 | −0.428 | −0.302 |
| EIF4G3 | 1.307 | 1.011 | 1.548 | 0.423 | 0.174 | 0.177 |
| ELAC1 | 0.467 | 1.584 | 0.031 | 1.362 | 0.042 | −1.144 |
| ELL2 | −0.8025 | 1.1815 | 0.7385 | 1.0585 | 0.9265 | 0.8125 |
| ELL3 | 0.0475 | 1.7675 | −0.4955 | −0.3395 | −0.4275 | −0.4745 |
| ELOVL5 | −0.6305 | −0.1245 | 0.1985 | 0.3455 | −0.5045 | 0.3855 |
| EN1 | 1.5235 | −1.9585 | −0.0145 | 0.3875 | −0.4415 | 0.3685 |
| ENAH | 0.2305 | 0.7295 | 1.3295 | 0.5845 | −0.4085 | 0.2075 |
| EPHX2 | 0.374 | −0.079 | −0.051 | 1.115 | −0.825 | −0.8 |
| EPLIN | −0.1315 | 0.9735 | −0.3625 | 0.8915 | −0.0485 | −0.7045 |
| EPN3 | −1.4995 | −0.5045 | −1.4605 | 0.6815 | −1.0075 | −1.3995 |
| EPOR | 0.226 | 0.195 | −0.967 | 0.098 | 0.347 | 0.267 |
| ERBB2 | 0.6335 | 3.3505 | 0.7955 | 2.8265 | 1.2925 | 1.7095 |
| ERP70 | −0.093 | 0.163 | 0.452 | 0.346 | −0.345 | 0.734 |
| ESR1 | 0.1945 | −0.4115 | 0.0155 | −0.4075 | 0.2825 | −0.0265 |
| ETFA | 0.2045 | −0.0105 | −0.0565 | −0.5515 | 1.2785 | 0.1095 |
| ETV8 | 0.2445 | 1.3255 | 1.0165 | 0.0265 | −0.1555 | −0.3845 |
| EVER1 | −0.5035 | 0.5605 | 0.1455 | 0.3275 | 0.2455 | −0.3595 |
| EXO1 | 0.3265 | −0.2935 | −0.6585 | −0.8965 | −0.9955 | −0.1045 |
| EXOSC9 | −0.131 | −0.644 | 0.588 | 0.443 | −0.581 | 0.248 |
| EXT2 | −0.111 | 0.331 | 0.047 | 0.946 | 0.415 | 0.465 |
| EZH2 | −0.1865 | −0.3915 | −0.4535 | 0.3175 | 0.1385 | −0.0175 |
| FI1R | 0.594 | 1.089 | 1.275 | 1.395 | −0.447 | −0.717 |
| F9 | −1.494 | −0.545 | −1.204 | −0.244 | 0.188 | −0.077 |
| FA2H | −0.094 | −0.284 | −0.806 | 0.103 | −0.331 | 0.28 |
| FABP7 | −0.447 | −0.029 | 0.702 | 0.728 | −0.507 | −0.409 |
| FADD | −0.408 | −0.337 | −0.442 | −0.489 | −0.074 | −0.163 |
| FAHD1 | 1.018 | 0.507 | 0.633 | 0.179 | 0.903 | 0.002 |
| FAHD2A | −0.265 | −0.149 | −0.386 | −0.262 | −0.1 | −0.422 |
| FAM38A | −0.2905 | −0.9485 | 3.2965 | 3.1555 | 1.7245 | 0.1665 |
| FAM3A | 0.0015 | 0.1755 | 0.0385 | −0.1185 | −0.8295 | −0.1355 |
| FAM3B | 0.303 | 0.794 | −0.414 | 0.777 | 1.263 | −0.092 |
| FAM3C | 0.529 | 0.023 | 0.502 | −0.011 | −0.369 | 0.005 |
| FAM46C | 0.414 | 0.692 | 1.352 | 0.644 | −0.333 | −0.701 |
| FAM49B | −0.445 | 1.517 | 0.677 | 0.057 | −0.294 | −0.038 |
| FAM54A | 0.212 | 0.718 | 0.362 | 0.985 | −0.137 | 0.159 |
| FANCA | −0.5205 | −0.4775 | −0.0375 | −0.1785 | −0.0575 | −0.4995 |
| FANCE | 1.1575 | 0.6925 | 1.2985 | 0.3555 | 0.2315 | 0.0825 |
| FBL | 0.174 | 0.364 | 0 | 0.116 | 0.347 | −0.143 |
| FBP1 | −0.356 | 0.06 | −0.52 | 0.453 | −0.028 | −0.089 |
| FBS1 | 0.6545 | −1.2155 | −0.0605 | 0.5225 | −0.5835 | 0.0485 |
| FBXL20 | −0.615 | 0.695 | 0.05 | −0.428 | 0.588 | −0.393 |
| FBXO25 | 0.431 | −0.393 | −0.307 | 0.57 | −0.631 | 0.117 |
| FDXR | 0.942 | −0.237 | 0.518 | 0.918 | 0.748 | 0.248 |
| FEM1B | 1.634 | 2.76 | −0.095 | −1.063 | −1.022 | −0.085 |
| FGF12 | 0.7045 | 0.6175 | 0.1265 | −0.2485 | 0.2295 | 0.4565 |
| FGF13 | 0.089 | 0.946 | 0.567 | 0.644 | 0.631 | −0.182 |
| FHOD1 | −1.4745 | −0.2705 | 0.3355 | −0.3145 | −0.2875 | 0.1675 |
| FKBP11 | 0.2155 | 0.6445 | 0.3905 | 0.3805 | 0.3845 | 0.1575 |
| FLJ10094 | 0.265 | −0.891 | −0.959 | −0.236 | −0.452 | −0.192 |
| FLJ10115 | −0.0065 | −0.4265 | −0.4755 | 0.2725 | 0.0555 | −0.6475 |
| FLJ10159 | −0.059 | 0.44 | 0.066 | 0.524 | −0.398 | −0.353 |
| FLJ10204 | 1.293 | 0.257 | 0.956 | 0.87 | −0.231 | −0.117 |
| FLJ10275 | −0.6825 | −0.8855 | 2.0585 | −0.1815 | −0.9775 | −0.2885 |
| FLJ10292 | −0.3555 | 0.1975 | −0.9165 | 0.8275 | −0.3575 | −0.3485 |
| FLJ10324 | 0.3515 | 0.3735 | 1.0855 | 0.3845 | 0.0725 | −0.1305 |
| FLJ10700 | 0.928 | 0.46 | −0.07 | 0.407 | −0.451 | 0.228 |
| FLJ10706 | 0.767 | −0.495 | −0.754 | 0.843 | −0.168 | −0.214 |
| FLJ10671 | −0.482 | −1.95 | −0.402 | −0.282 | −1.282 | −0.317 |
| FLJ10901 | 0.8845 | 1.0205 | 0.4015 | 2.4265 | 0.4125 | 1.1025 |
| FLJ10918 | −0.713 | 0.588 | −0.357 | 1.816 | 0.182 | 0.161 |
| FLJ10980 | −0.0145 | 0.0205 | 0.3255 | 0.6135 | −0.1876 | −0.5075 |
| FLJ11017 | 0.3665 | −0.0735 | 0.6835 | −0.4805 | 0.0795 | −0.8415 |
| FLJ11088 | −0.6465 | 0.0205 | −0.7405 | −0.1415 | 0.2155 | −0.1525 |
| FLJ11151 | 0.7155 | 0.1035 | 0.1665 | 0.2715 | 1.4555 | 0.3705 |
| FLJ11267 | 0.3605 | 0.1105 | 0.4876 | 0.2325 | 0.4705 | 0.1145 |
| FLJ11280 | −0.4445 | 0.2015 | −0.4875 | −0.1335 | −0.0655 | −0.6015 |
| FLJ11508 | 0.9816 | 1.2105 | 0.3495 | 0.1835 | 0.0235 | 1.1425 |
| FLJ12270 | −0.8765 | −0.6885 | −1.2905 | 0.1455 | 1.3935 | 2.2895 |
| FLJ12844 | 0.0845 | −0.4085 | 0.5835 | 0.6785 | −0.3045 | −0.3175 |
| FLJ12650 | −0.0535 | 0.1705 | 0.1965 | 0.2435 | −0.0655 | −0.2905 |
| FLJ12684 | 0.0885 | −0.0535 | 0.0085 | −0.0945 | 0.1365 | −0.1395 |
| FLJ12735 | −0.434 | −1.204 | −0.288 | 0.984 | 0.679 | 0.41 |
| FLJ12750 | 0.533 | 1.008 | −0.414 | 2.222 | 0.671 | −0.15 |
| FLJ12895 | −0.1715 | 0.3216 | 0.0355 | 0.8985 | −0.0565 | −0.4875 |
| FLJ13710 | −0.2695 | 0.9416 | 0.5235 | 0.5255 | 0.1315 | 0.4265 |
| FLJ13855 | −0.133 | −0.768 | 0.232 | 0.176 | 0.347 | −0.018 |
| FLJ13912 | −0.005 | 0.324 | 0.241 | 0.034 | 0.034 | −0.299 |
| FLJ14124 | −0.182 | 0.535 | 0.288 | 0.394 | 0.044 | −0.003 |
| FLJ14627 | 0.1065 | −0.3025 | 0.0825 | 0.0265 | −0.1985 | 0.0015 |
| FLJ14666 | 1.561 | 2.037 | 1.023 | 0.095 | 0.275 | −0.624 |
| FLJ14904 | 2.51 | 1.441 | 1.832 | 0.902 | 0.364 | 0.318 |
| FLJ20035 | −0.296 | 0.153 | −0.858 | 0.308 | 0.036 | −0.172 |
| FLJ20152 | −0.208 | −0.208 | −0.294 | 0.194 | 0.277 | −0.345 |
| FLJ20171 | 0.567 | 1.041 | 0.682 | 0.757 | 0.015 | −0.445 |
| FLJ20244 | −0.075 | 0.92 | 0.34 | 0.666 | −0.714 | −0.949 |
| FLJ20273 | 1.441 | 0.356 | 0.523 | −0.327 | 0.254 | 0.635 |
| FLJ20315 | 0.509 | −0.009 | 0.573 | 0.798 | −0.011 | 0.171 |
| FLJ20323 | 0.4145 | −0.1535 | 0.1825 | 0.0205 | −0.5565 | −0.3805 |
| FLJ20518 | 0.701 | 0.091 | 1.159 | 0.244 | 1.12 | 0.238 |
| FLJ20530 | −0.182 | 0.678 | 0.018 | −0.11 | −0.093 | −0.09 |
| FLJ20696 | 0.3155 | 0.0035 | −0.8875 | −0.5065 | 0.1385 | −0.5835 |
| FLJ20718 | −0.398 | −0.327 | −0.534 | −0.072 | −0.234 | −0.245 |
| FLJ20772 | −0.7565 | 1.1055 | 0.0595 | 1.4445 | −0.1055 | 0.2295 |
| FLJ20989 | 0.3735 | 1.0275 | −0.1945 | 0.6855 | −0.1385 | 0.2475 |
| FLJ21019 | −1.4945 | −1.5026 | −1.4505 | −1.6325 | −0.1035 | −1.3215 |
| FLJ21062 | 0.3735 | 1.4445 | −0.5885 | 1.0575 | 0.3705 | 0.6085 |
| FLJ21159 | −0.4815 | 0.6455 | −0.3495 | −0.1915 | 0.9345 | 0.5055 |
| FLJ21616 | 1.284 | 0.407 | 0.665 | −1.45 | −0.574 | 0.044 |
| FLJ21827 | 0.1685 | 0.5445 | 0.1265 | −0.0135 | 0.0005 | 0.3365 |
| FLJ21963 | −0.536 | 0.243 | 0.268 | −0.285 | 0.77 | −0.272 |
| FLJ22104 | 0.7245 | 1.1215 | 1.2695 | −0.3625 | 0.3775 | −0.4265 |
| FLJ22222 | 0.377 | 0.583 | 0.782 | −0.051 | 0.753 | 0.755 |
| FLJ22573 | 0.3625 | 1.9975 | 1.1555 | 2.9035 | 0.1645 | 1.1315 |
| FLJ22794 | 0.147 | 0.13 | −0.045 | −0.194 | −0.299 | −0.126 |
| FLJ23188 | −0.4635 | 0.0295 | −0.6785 | −0.3145 | −0.5745 | −0.4385 |
| FLJ23441 | 0.58 | 0.61 | 0.584 | 0.844 | 0.043 | −0.454 |
| FLJ25471 | −0.269 | 0.572 | −0.295 | 1.239 | −0.335 | −0.315 |
| FLJ31204 | −0.4295 | 0.4325 | −0.1275 | 0.0735 | 0.5735 | 0.3065 |
| FLJ31795 | 0.6385 | 1.0425 | 1.1335 | −0.0525 | 1.1495 | 0.1175 |
| FLJ32942 | −0.1245 | 1.4545 | 2.0675 | 0.8065 | −0.2885 | 0.1205 |
| FLJ37970 | −3.124 | −3.039 | −2.835 | −0.068 | −1.706 | −2.246 |
| FLJ39370 | −0.7955 | 0.2055 | 0.2465 | 0.1385 | 0.8005 | −0.4705 |
| FLJ90588 | −0.9925 | −0.6515 | −0.6295 | −0.0025 | 0.0025 | −0.0725 |
| FN3KRP | 0.578 | −0.036 | 0.094 | 0.674 | 0.498 | 0.5 |
| FOLR1 | −1.5095 | −1.6395 | −2.0185 | −0.9245 | −0.1405 | −0.1895 |
| FOXA1 | 0.4805 | 0.4785 | 1.5645 | 0.2705 | 0.0245 | −0.0395 |
| FOXC1 | −0.976 | 0.077 | 0.011 | −1.019 | −0.604 | −0.318 |
| FOXP1 | −0.5285 | 0.1305 | 1.3715 | −0.3995 | −0.2175 | −0.3315 |
| FREQ | 0.2116 | 0.3485 | −0.0525 | 0.2245 | 0.2815 | 0.5795 |
| FTO | 0.5205 | 0.3395 | 0.6805 | −0.1025 | 0.2285 | 0.4245 |
| FUBP1 | 1.6485 | 0.0025 | 0.4915 | 0.8175 | −0.3785 | 0.2215 |
| FUCA1 | −0.75 | −1.153 | −0.488 | −0.164 | −0.39 | −0.077 |
| FUT8 | −0.3125 | 0.4455 | 0.2585 | 0.4325 | 0.1055 | 0.2065 |
| FXYD6 | 0.49 | −0.362 | −0.968 | −0.26 | 0.284 | 1.201 |
| FYCO1 | −0.339 | −0.82 | −0.221 | 0.644 | −0.6 | 0.151 |
| FZD10 | −0.3885 | 0.3405 | −0.0315 | −0.3525 | −0.2385 | −0.2535 |
| FZD7 | 1.4055 | 2.9185 | 0.0085 | −0.5905 | 0.9185 | −0.5115 |
| FZD9 | 1.4635 | 1.8085 | −0.3195 | 0.1185 | 1.1305 | 0.0255 |
| G1P2 | 0.3715 | −0.1665 | −0.5315 | 0.2795 | −0.0065 | 0.0355 |
| G1P3 | −0.251 | 0.095 | −0.091 | −0.373 | −0.549 | −0.324 |
| GA17 | −0.1665 | 0.5485 | 0.6915 | 0.4485 | −0.6945 | −1.3345 |
| GABARAP | 0.9355 | 0.0815 | −0.9005 | 0.0895 | 0.1245 | 0.2215 |
| GAJ | 1.3745 | −0.4285 | −1.1925 | 0.0225 | −0.4695 | −0.3505 |
| GALE | 0.888 | −0.169 | −0.301 | −0.154 | 0.383 | 0.544 |
| GALNT14 | 1.2355 | 0.3755 | 1.0825 | 1.2965 | −0.6875 | −0.3175 |
| GALNT2 | 0.2275 | −1.5305 | −0.7275 | 0.3355 | 1.1045 | −0.0945 |
| GALNT7 | 0.145 | −0.161 | −0.371 | −0.049 | −0.166 | 0.038 |
| GAMT | 0.039 | −1.111 | 0.226 | −0.778 | 0.193 | 0.001 |
| GAPD | 0.208 | 0.169 | −0.12 | −0.010 | 0.123 | −0.011 |
| GARS | 0.095 | 0.409 | −0.062 | 0.087 | 1.177 | 0.258 |
| GART | 1.0425 | −0.6345 | 1.6345 | 1.3945 | −0.6655 | 0.0005 |
| GATA3 | 0.1115 | −0.8385 | −0.4295 | −0.4815 | 0.0495 | −0.1255 |
| GATM | 1.512 | −0.852 | −0.778 | −0.201 | 1.013 | −0.509 |
| GBA | 0.214 | −0.023 | 0.109 | −0.074 | 0.265 | −0.083 |
| GDAP1 | −0.428 | 0.473 | 0.118 | −0.609 | −0.113 | −0.796 |
| GDI2 | 0.6755 | 0.2685 | 0.7745 | 0.2615 | 0.1095 | −0.0585 |
| GGA1 | 0.7715 | 0.9945 | 0.3895 | −0.8785 | 0.3825 | 0.5755 |
| GKAP1 | −0.0065 | −0.1105 | −0.3915 | −0.6415 | −0.2735 | −0.2275 |
| GLCCI1 | −0.0775 | −0.5075 | −0.2885 | −0.2655 | 0.2705 | −0.0045 |
| GLG1 | −0.417 | −0.242 | 0.056 | −1.08 | −0.281 | −0.003 |
| GLTSCR2 | 1.838 | 0.783 | −0.801 | 0.837 | 0.071 | −0.03 |
| GNB4 | 0.294 | −0.177 | 0.213 | 0.112 | −0.05 | −0.083 |
| GOLPH2 | −0.5905 | −0.1615 | −0.3665 | −0.1935 | 0.2135 | 0.1105 |
| GPATC1 | −1.1815 | −0.3295 | −1.1585 | −1.5775 | −0.2465 | −0.2075 |
| GPC2 | 1.2655 | 0.8585 | 2.2425 | 1.3415 | −0.4165 | 0.0435 |
| GPR125 | 0.571 | 0.298 | −0.758 | 0.349 | −0.68 | −0.765 |
| GPR160 | 0.3495 | 0.2855 | 0.6865 | 0.1905 | 0.1775 | −0.1525 |
| GPR56 | −1.1125 | −0.0035 | −1.4205 | −0.0725 | 0.3295 | −0.0125 |
| GPRC5C | −0.2075 | 0.4825 | 0.9085 | 0.2925 | −0.0655 | −0.2355 |
| GPSM1 | −1.825 | −1.044 | 0.691 | −0.688 | −0.333 | 0.476 |
| GPSM2 | −1 | −0.117 | −2.065 | 1.153 | −0.797 | 0.19 |
| GPT | −0.5235 | −0.2925 | −0.4275 | −0.8955 | −0.7785 | −0.0465 |
| GPT2 | −0.369 | 2.508 | −1.062 | 4.804 | 1.917 | 1.209 |
| GPX7 | −0.631 | −0.149 | −0.24 | −0.389 | −0.455 | −0.431 |
| GRB14 | 1.1835 | 1.4945 | −1.1705 | 0.2585 | −0.1795 | 1.3095 |
| GRB7 | −0.3535 | 0.3725 | 0.0595 | 0.5275 | 0.2575 | −0.0275 |
| GSC | −0.373 | −0.184 | −0.075 | −0.115 | 0.129 | −0.185 |
| GSDML | −0.266 | −0.574 | −0.31 | −0.321 | −0.209 | −0.151 |
| GSK3B | −0.074 | 0.13 | −0.891 | −0.26 | −0.304 | −0.564 |
| GSTA1 | −1.0025 | 0.3035 | 0.7615 | 2.3855 | −0.8275 | 0.7445 |
| GSTK1 | 1.963 | 1.526 | −1.273 | −1.672 | −0.323 | −0.849 |
| GSTM3 | −2.1945 | −1.3805 | 0.4185 | 0.0935 | −1.4315 | 0.2235 |
| GSTP1 | −1.007 | 1.609 | −0.715 | 0.092 | 0.043 | −0.238 |
| GSTT1 | −0.4615 | 0.0435 | −0.2755 | 0.3905 | −0.0415 | −0.1535 |
| GSTT2 | −0.1645 | 0.0865 | −0.3375 | 0.3555 | 0.2045 | −0.1465 |
| GTF2F2 | −0.358 | 0.502 | 0.329 | −0.272 | 0.084 | 0.31 |
| GTF2I | −0.3165 | 0.0065 | −0.3425 | −0.3645 | 0.1255 | −0.6995 |
| GTF3A | 0.768 | −0.018 | 0.061 | −0.063 | −0.178 | −0.4 |
| GTPBP3 | −1.061 | 0.838 | −0.032 | 0.861 | 0.003 | 0.618 |
| GTPBP4 | 0.332 | 0.905 | 1.768 | 0.572 | 0.332 | 0.475 |
| H1F0 | 0.6885 | 0.0565 | 0.3405 | −0.2325 | 0.8445 | 0.0065 |
| H2AFV | −0.3505 | 1.1165 | 0.5105 | 0.4985 | 0.1385 | −0.2705 |
| H2AFY | 1.2605 | −8.5055 | 0.8945 | 1.1225 | 0.3585 | 1.5175 |
| H2AFZ | −0.7755 | 0.7595 | 0.1575 | 0.3505 | 0.4785 | −0.8655 |
| H2BFS | −0.4985 | 0.8845 | 8.5675 | 0.3505 | 0.5885 | 0.1375 |
| H3F3B | −0.495 | 0.379 | 0.276 | −0.085 | 0.691 | −0.304 |
| H41 | −0.1925 | 0.5595 | 0.1735 | 0.1065 | 0.3565 | 0.0755 |
| HAGH | 0.308 | 1.069 | −0.55 | 0.672 | 0.205 | −0.006 |
| HBG1 | −0.3135 | 0.4505 | 0.7765 | 0.0425 | −0.0445 | −0.3305 |
| HBL01 | 0.774 | 1.998 | 0.827 | −0.667 | 1.19 | −0.417 |
| HCAP-G | 0.2815 | 0.6075 | 0.2525 | 0.4335 | 0.1655 | −0.5595 |
| HCP5 | −0.481 | 0.157 | 0.854 | 1.17 | 0.458 | −0.16 |
| HDAC2 | 1.316 | −0.289 | 0.322 | −0.335 | −0.64 | −0.465 |
| HDC | 0.0005 | −0.2775 | 0.4215 | 0.0085 | −0.2955 | −0.2165 |
| HEBP1 | 0.419 | 0.718 | −0.735 | 0.352 | 0.821 | −0.834 |
| HIPK2 | 0.129 | 0.346 | 0.187 | 1.826 | 0.162 | 0.315 |
| HIPK2 | 1.2465 | −1.3555 | 0.3735 | −0.3755 | 0.3605 | 1.4265 |
| HIS1 | 1.0755 | −1.1065 | 0.6065 | 0.4445 | 0.2485 | 1.5555 |
| HIST1H2A | 0.997 | −1.146 | 0.623 | 0.475 | 0.35 | 1.344 |
| HIST1H2B | 1.0235 | −1.1855 | 0.5755 | 0.4895 | 0.3225 | 1.0895 |
| HIST1H2B | 1.037 | −1.215 | 0.683 | 0.468 | 0.343 | 1.235 |
| HIST1H2B | −0.8025 | 0.2455 | 0.1955 | 0.1135 | −0.0065 | −0.6725 |
| HIST1H2B | 1.6225 | 0.2105 | 0.0565 | −0.0775 | 0.2755 | −0.4935 |
| HIST1H3C | −0.6045 | −0.1435 | −0.2875 | −0.5445 | 0.0345 | −0.2255 |
| HM13 | −1.1615 | 0.0765 | 0.3175 | −0.6655 | 0.4475 | 0.0475 |
| HMGB3 | 0.3375 | 0.7695 | 0.6625 | 0.3275 | 0.0865 | −0.2785 |
| HN1 | 0.078 | 0.494 | 0.042 | 0.2 | 0.027 | −0.055 |
| HNRPA3P | 0.1655 | −0.8455 | 0.0265 | −0.8125 | −0.0415 | 0.2965 |
| HNRPDL | −0.236 | −1.439 | −0.269 | −0.685 | −0.097 | 0.48 |
| HOXA5 | 1.7315 | 0.4635 | 2.6846 | −2.4025 | 0.3585 | 0.3175 |
| HOXA7 | 2.3615 | 0.0745 | 3.3795 | −0.4215 | −0.6165 | 0.3165 |
| HOXB2 | 0.7105 | 0.1345 | 0.8405 | −0.1585 | −0.5285 | −0.4715 |
| HOXB6 | 1.1325 | −0.3395 | 1.0355 | −0.3315 | 0.7535 | −0.1365 |
| HOXB7 | 1.1295 | 0.0155 | 0.1035 | −0.1215 | 0.1275 | 0.3875 |
| HOXC10 | −1.214 | 0.283 | −0.089 | −0.097 | 0.481 | 0.519 |
| HOXC13 | −2.1635 | −0.7605 | 0.7775 | −0.2505 | −0.0255 | 0.5645 |
| HOXC9 | −0.14 | 0.358 | 0.836 | −0.077 | 0.056 | −0.105 |
| HOXD3 | 0.0965 | −0.0055 | 0.4685 | 0.3085 | −0.3265 | −0.1985 |
| HPCL2 | −1.749 | 0.379 | −1.418 | 1.798 | −0.928 | 0.058 |
| HPS3 | 0.7145 | 0.1645 | 0.5435 | −0.3675 | −0.3795 | −0.0415 |
| HRASLS | 0.6255 | 0.4565 | −0.8655 | 0.4145 | −0.4975 | −0.5075 |
| HRB | 0.856 | −0.141 | −0.801 | 0.111 | −0.153 | −0.167 |
| HRSP12 | −0.435 | −0.083 | −0.677 | −0.16 | −0.4 | −0.468 |
| HSD17B2 | 0.8625 | −0.0935 | 0.7805 | 0.3355 | −0.2405 | −0.1795 |
| HSF1 | −1.0715 | −0.8526 | −0.3205 | 0.6015 | 0.3865 | −0.4565 |
| HSPA14 | 0.122 | −0.174 | −0.64 | −0.678 | 0.139 | 0.064 |
| HSPA2 | −0.0275 | 0.8285 | 0.7715 | 0.7415 | 0.2675 | 0.3625 |
| HSPA5BP [symbol] | 0.617 | −0.355 | −0.543 | −0.644 | −0.058 | 0.495 |
| HSPC016 | 0.328 | 0.184 | −0.272 | −0.292 | 0.108 | 0.012 |
| HSPC138 | −0.163 | 0.387 | 1.207 | 0.07 | 0.112 | −0.439 |
| HSPC148 | 1.6625 | −0.1085 | 0.9045 | −0.0135 | −0.2875 | 0.0955 |
| HSPC150 | −0.3965 | 0.2935 | 0.4885 | −0.3635 | 0.1725 | −0.4405 |
| HSPC163 | −0.481 | −0.281 | −0.478 | −0.592 | 0.016 | −0.398 |
| HSPC266 | −0.079 | −0.201 | 0.387 | −0.272 | −0.793 | −0.292 |
| HSU79274 | −0.858 | 0.231 | −0.405 | −0.618 | −0.144 | 0.414 |
| HTATIP2 | −0.437 | −0.168 | 0.058 | 0.319 | 0.341 | −0.638 |
| ICMT | −1.19 | −1.528 | −1.82 | 0.455 | −0.42 | −0.423 |
| ICT1 | 0.627 | 0.777 | 1.391 | −0.636 | −0.418 | 0.164 |
| ID4 | −0.3005 | 0.0895 | 0.3185 | −0.6985 | −0.2905 | 0.0345 |
| IDH2 | −0.268 | 0.016 | 1.403 | −1.238 | 0.051 | −0.485 |
| IDS | 2.0905 | 2.0745 | 0.9025 | 0.4355 | 0.4165 | −0.3055 |
| IFI30 | 0.7205 | 1.0365 | 0.8945 | −0.1435 | −0.0995 | −0.5625 |
| IFI44 | 0.5885 | 1.4675 | 0.3115 | 0.1525 | 0.2865 | 0.0265 |
| IFIT5 | −0.1755 | −0.0535 | −0.0585 | 0.2425 | −0.2495 | −0.0365 |
| IFITM1 | 0.8495 | −0.1495 | 0.8405 | −0.1145 | −0.4215 | 0.1615 |
| IGBP1 | −0.7145 | 0.3285 | 1.9505 | 0.8425 | 0.8175 | 0.3345 |
| IGFALS | 1.3905 | 1.5245 | 0.8545 | 0.2915 | 1.5225 | −0.1755 |
| IGPBP2 | 1.7825 | 1.2185 | 0.8595 | 0.5495 | 2.0305 | −0.2385 |
| IGHG1 | 1.1715 | 0.8295 | 0.5065 | 2.4815 | 1.5705 | 0.5795 |
| IGHG1 | 1.432 | 1.721 | 1.213 | 0.32 | 2.76 | −1.029 |
| IGJ | 1.031 | 1.216 | 0.373 | 0.306 | 1.053 | −0.568 |
| IGKC | 1.189 | 1.593 | 0.501 | 0.074 | −0.18 | −0.362 |
| IGKC | 1.348 | 3.761 | 0.043 | −0.202 | 1.718 | −0.426 |
| IGLC2 | 0.3075 | −0.0005 | 0.3505 | −0.2855 | −0.0505 | 0.4345 |
| IGLL1 | −0.5115 | 0.1175 | −0.3045 | 1.6895 | 0.6635 | 0.2115 |
| IL13RA1 | 0.495 | 1.014 | 1.008 | 0.535 | 0.171 | −0.148 |
| IL17B | 0.6075 | −0.1235 | −0.4075 | 1.0255 | 1.4325 | 0.2935 |
| IL23A | −0.0975 | −0.2085 | 0.1085 | 0.0695 | −0.2115 | 0.1825 |
| IL6ST | −1.1225 | −0.2695 | −0.1825 | 0.3955 | −0.5415 | −0.6385 |
| ILF2 | −0.07 | 0.332 | 0.043 | 0.798 | 0.32 | 0.282 |
| IMPA2 | −0.7415 | −2.3995 | 0.2775 | −0.1885 | −1.4745 | 0.7385 |
| ING1L | −0.511 | −0.993 | −0.473 | −0.284 | 0.311 | 0.653 |
| INHBB | 0.4515 | 0.5705 | −0.2095 | −0.7575 | 1.8975 | −0.2585 |
| INPP4B | 0.4955 | −0.6805 | −2.1335 | 1.1035 | 1.3395 | 0.3835 |
| INSM1 | 0.59 | 0.418 | 1.15 | 0.208 | 0.476 | 1.149 |
| IQCA | 2.649 | −0.511 | 2.469 | −0.367 | −0.361 | 0.702 |
| IQCE | 0.948 | 1.679 | 0.879 | 0.288 | 1.556 | 0.436 |
| IQGAP2 | 1.5675 | 2.1955 | 0.9705 | 0.9855 | 1.9915 | −0.7465 |
| IRF4 | −0.7465 | 1.2885 | −2.4645 | −0.0025 | −0.7715 | −0.8835 |
| IRTA2 | 0.767 | 0.981 | 0.576 | 0.234 | 0.838 | −1.048 |
| IRX4 | 0.8715 | 0.8805 | 0.0535 | 0.5535 | 0.9995 | 0.0075 |
| ISG20 | −0.733 | −0.088 | −0.798 | 0.289 | −0.201 | −0.533 |
| ISGF3 [symbol] | 0.505 | 0.979 | 0.295 | −0.025 | −1.04 | −0.646 |
| ITGB4 | 0.028 | −0.387 | −0.006 | −0.613 | 0.552 | −0.088 |
| ITPR1 | −0.173 | 0.678 | −1.609 | 0.617 | 0.517 | 0.113 |
| JARID1A | 0.32 | 0.041 | −0.536 | −0.084 | −0.147 | −0.172 |
| KARCA1 | −0.003 | 0.126 | −0.13 | 0.017 | 0.02 | 0.186 |
| KATNA1 | −0.5105 | 0.5395 | −0.7875 | 1.0425 | 0.6535 | −0.0585 |
| KCNC2 | 0.002 | 0.122 | 0.27 | −0.132 | −0.304 | −0.386 |
| KCND2 | 0.84 | −0.837 | 0.087 | −0.736 | −0.115 | −0.919 |
| KCNK4 | 1.4 | 0.172 | 0.277 | −0.398 | −0.638 | −0.582 |
| KCNN4 | 0.2495 | 0.0085 | −2.2245 | 0.0355 | 0.4355 | −0.1405 |
| KCNS3 | 0.2425 | 0.3195 | 0.3876 | 1.1065 | 0.0125 | 0.0765 |
| KCTD15 | 0.854 | 0.314 | 1.437 | 1.096 | −1.363 | 1.477 |
| KCTD3 | 0.656 | −0.268 | −0.248 | 0.33 | 0.055 | −0.118 |
| KDELR2 | 0.275 | 0.95 | 1.748 | 0.909 | −0.19 | 0.778 |
| KIAA0020 | −1.08 | −0.257 | −0.521 | −1.246 | −0.52 | −0.357 |
| KIAA0101 | −0.4245 | 1.3605 | 0.2945 | 0.1365 | 0.1895 | 0.6725 |
| KIAA0153 | 0.0585 | 0.0735 | −0.2225 | −0.2385 | −0.5525 | −0.4245 |
| KIAA0182 | −0.2625 | −0.1315 | 0.8015 | −0.0035 | 0.1725 | 0.7435 |
| KIAA0196 | 0.8875 | 0.0695 | 0.9945 | 1.1045 | −0.1225 | 0.1745 |
| KIAA0241 | 0.164 | 1.28 | 0.438 | −0.001 | 0.788 | −0.062 |
| KIAA0251 | −0.3515 | −0.1845 | 0.3855 | −0.5835 | 0.1045 | −0.0455 |
| KIAA0274 | −0.418 | −1.026 | −0.17 | −0.155 | −0.196 | −0.803 |
| KIAA0515 | 0.5475 | 0.5335 | 0.5135 | 0.4415 | 0.1605 | 0.1435 |
| KIAA0582 | 1.9975 | 0.6755 | 1.5425 | 0.8905 | −0.5555 | −0.3275 |
| KIAA0650 | 0.3685 | 0.7605 | −1.4925 | 0.9045 | 0.0085 | −0.9455 |
| KIAA0703 | −1.306 | 0.032 | −0.484 | −0.146 | −0.768 | −0.324 |
| KIAA0830 | 2.0635 | 0.8395 | 1.1345 | 1.4735 | 0.3045 | 0.2015 |
| KIAA0980 | −0.1115 | 0.3295 | 0.5645 | −0.2715 | −0.4885 | −0.4535 |
| KIAA1324 | 0.51 | 0.409 | −0.181 | −0.161 | 0.208 | 0.136 |
| KIAA1363 | 1.47 | 1.285 | 1.031 | 0.931 | 0.259 | 1 |
| KIAA1683 | −0.4835 | 2.1585 | 0.3875 | 1.7775 | 0.1525 | 0.2355 |
| KIF12B | 0.4645 | −0.1705 | −0.1945 | 0.1995 | 0.1205 | −0.0315 |
| KIF24 | 0.0065 | 0.0195 | 0.2455 | −0.1475 | 0.3185 | 0.0745 |
| KIN | −1.7485 | −1.2275 | −1.3405 | 0.8875 | −0.2155 | −0.1375 |
| KIRREL3 | −0.4985 | −0.6845 | −0.7745 | 0.5805 | −0.3805 | −0.6965 |
| KIT | −1.8505 | −1.4865 | −1.8575 | −0.0085 | −0.1015 | −0.4725 |
| KLK5 | −0.755 | −1.078 | −0.309 | 1.204 | −0.283 | −0.728 |
| KLK6 | −1.304 | −1.079 | −1.002 | 0.102 | 0.006 | −0.464 |
| KLK7 | −0.312 | 0.504 | 1.001 | −0.056 | 0.308 | −0.015 |
| KLK8 | −0.8175 | 0.3945 | 0.8045 | 0.1505 | 0.2525 | −0.7905 |
| KNTC2 | −0.078 | 0.54 | −0.14 | −0.092 | 0.61 | 1.13 |
| KPNA2 | 0.1955 | 0.2395 | −0.0825 | 0.6005 | −0.3915 | 0.3955 |
| KREMEN2 | −0.7375 | −0.2505 | −1.1985 | 3.2055 | −0.0405 | −0.3915 |
| KRT10 | −0.9525 | 1.3345 | −2.8845 | 3.0415 | −0.3995 | −0.3445 |
| KRT13 | −2.069 | −1.578 | −1.801 | 0.968 | 0.34 | −0.889 |
| KRT15 | 0.135 | 0.81 | −0.141 | 1.135 | 0.88 | 0.023 |
| KRT17 | −0.3105 | −2.6585 | −1.2585 | −0.4965 | −1.8795 | 0.3515 |
| KRT18 | −2.487 | −2.083 | −2.223 | 0.349 | −0.534 | −1.526 |
| KRT23 | 2.3095 | 0.0305 | −0.0685 | −0.1665 | 0.0365 | −0.3205 |
| KRT6B | −0.086 | −0.051 | 0.015 | 0.025 | −0.18 | 0.184 |
| KRTAP3-3 | −0.1125 | −0.1975 | 0.0015 | 1.8825 | −0.0245 | 0.0485 |
| KRTHA7 | 0.8145 | −2.4055 | −2.8075 | 0.4845 | −0.2955 | −0.0955 |
| LAMA3 | 0.439 | −0.151 | −0.003 | −0.19 | 0.431 | 0.318 |
| LAMB3 | 0.934 | 1.64 | 0.591 | 0.287 | 0.339 | −0.198 |
| LASS2 | −0.0545 | −0.3815 | 0.6975 | 0.3585 | 0.1485 | 0.1985 |
| LAX | −2.1225 | −0.7455 | 0.5765 | −1.3575 | −1.2525 | 1.4375 |
| LBR | −0.5465 | −1.3455 | 0.2545 | −1.1305 | −0.3175 | −0.3245 |
| LCE1B | −0.7655 | 0.7035 | 0.1635 | 0.4835 | −0.4435 | −0.5485 |
| LDHB | −0.226 | 0.004 | 0.101 | 0.255 | −0.211 | 0.21 |
| LDOC1L | −0.357 | 0.439 | −0.87 | 1.798 | 0.467 | 0.267 |
| LETMD1 | 1.4035 | 0.2365 | 2.1695 | 0.2775 | 2.2065 | 0.4315 |
| LFNG | −0.259 | −0.392 | 0.086 | 0.094 | −0.012 | 0.063 |
| LGALS2 | 0.481 | 0.388 | −0.481 | 0.522 | −0.371 | −0.483 |
| LGTN | −0.566 | 1.291 | 0.966 | −0.305 | 0.458 | −0.52 |
| LNX | 0.4735 | 0.3335 | −0.3235 | 0.3525 | −0.0345 | 0.1715 |
| LOC11247 | 1.058 | 0.994 | 0.4 | 0.395 | 0.005 | −0.447 |
| LOC11492 | −0.008 | 0.112 | 0.706 | 0.084 | 0.264 | 0.361 |
| LOC11497 | −0.2465 | −0.1775 | 0.0345 | −0.3025 | −0.2615 | −0.3145 |
| LOC11621 | −0.1135 | 0.7285 | 0.0745 | −1.1445 | 0.0285 | 0.2975 |
| LOC11644 | 1.3305 | 0.1475 | 1.1845 | 0.7915 | 0.6635 | 0.6375 |
| LOC12022 | −0.0875 | −0.1495 | −0.0585 | −0.2575 | 0.1435 | −0.1895 |
| LOC12422 | −0.2475 | 0.3555 | −1.0405 | 0.5205 | −0.2085 | 0.5295 |
| LOC14654 | 0.634 | 0.067 | 0.178 | 0.971 | 0.333 | 0.283 |
| LOC15506 | 0.796 | 0.148 | −0.338 | 0.446 | −0.392 | −0.445 |
| LOC20189 | 3.6735 | −0.7785 | −0.1675 | −0.4765 | −0.3885 | 0.0685 |
| LOC25398 | 0.215 | 0.512 | −0.242 | −0.278 | −0.029 | 0.008 |
| LOC33990 | 0.445 | 0.109 | −0.408 | −0.007 | 0.158 | −0.557 |
| LOC34817 | −1.3215 | −0.2905 | −1.4155 | −0.4125 | −0.5795 | 0.0875 |
| LOC40045 | 1.612 | 1.034 | 0 | 1.98 | −0.061 | 0.699 |
| LOC44089 | 0.149 | 0.077 | −0.535 | −0.052 | 0.01 | −0.412 |
| LOC49386 | −1.202 | 0.017 | −0.585 | 0.122 | 0.046 | 0.118 |
| LOC51123 | −0.782 | 0.022 | 0.113 | 0.331 | −0.018 | −0.816 |
| LOC51161 | 0.9925 | −0.1305 | −0.5805 | 0.7925 | 1.3315 | −0.3485 |
| LOC51321 | 0.1835 | −0.3595 | 0.3075 | 1.2585 | 0.1055 | −0.3025 |
| LOC51760 | −0.0555 | 1.0815 | −0.1135 | 0.0755 | 0.3765 | 0.2935 |
| LOC81558 | 1.447 | 0.582 | 1.165 | 0.043 | 0.064 | −0.016 |
| LOC81569 | 0.7185 | 0.2625 | 0.2725 | −0.0765 | 0.2915 | −0.4655 |
| LOC90355 | 0.439 | 0.617 | −0.1 | 0.19 | −0.242 | 0.255 |
| LOC90701 | −0.223 | 0.856 | 2.181 | 0.24 | −1.194 | −1.523 |
| LONP | 0.2485 | 1.0245 | 0.0535 | 0.5945 | 0.2345 | 0.0015 |
| LRAP | −0.3505 | 0.9725 | 0.3825 | 0.7555 | 0.0985 | 0.1255 |
| LRBA | 0.0465 | 0.2015 | −0.4705 | −0.2925 | −0.6555 | 0.1095 |
| LRIG1 | −0.9385 | −0.2725 | −0.2195 | 0.2705 | −0.9035 | −0.8225 |
| LRP6 | −1.262 | 0.713 | −1.02 | −0.559 | −0.339 | 1.354 |
| LRP8 | 0.38 | 0.239 | 1.706 | 0.356 | 0.503 | 0.292 |
| LRRC17 | 1.1065 | 0.4095 | −0.2795 | 0.4135 | −0.2805 | 0.0775 |
| LRRC2 | 1.3985 | 0.6305 | 1.3255 | 0.1715 | 0.3795 | −0.0755 |
| LSM1 | 2.6605 | 0.4425 | 2.7115 | 1.3365 | 2.3815 | 0.2335 |
| LSM8 | −0.28 | −0.175 | 0.101 | −0.023 | −0.277 | −0.122 |
| LTB | 0.932 | −0.504 | 1.163 | −0.812 | −0.328 | −0.8 |
| LY6D | −0.215 | 0.805 | 0.865 | 0.429 | −0.505 | −0.685 |
| LYN | 0.512 | 0.053 | −0.009 | 0.295 | 0.066 | −0.005 |
| MA02L1 | −0.083 | −0.195 | −0.199 | −0.347 | 0.344 | 0.188 |
| MADP-1 | −0.5605 | 0.0775 | 0.0835 | 0.0565 | 0.4065 | 0.1735 |
| MAGEA12 | −0.258 | 0.027 | 0.32 | −0.03 | 0.334 | 0.185 |
| MAGEA2 | −0.7335 | −0.1785 | 0.0105 | 0.2295 | 0.4595 | 0.6255 |
| MAGEA3 | −1.2175 | −0.4535 | −1.4805 | 0.6415 | −0.1105 | 0.0075 |
| MAGEA4 | −0.575 | −1.182 | −0.3 | 1.453 | 0.5 | −1.305 |
| MAGED4 | −0.3075 | 0.9855 | −0.3645 | 0.3305 | 0.0025 | 0.4845 |
| MAGI1 | 0.4345 | −0.7455 | −1.0935 | 0.6235 | −0.3665 | −0.6895 |
| MAGI-3 | 0.444 | 0.013 | 0.675 | −0.182 | −0.224 | −0.223 |
| MAL2 | −1.038 | 0.231 | 1.745 | −0.922 | −0.818 | −0.815 |
| MAN2B1 | −0.7915 | −0.9585 | 0.2205 | −0.3505 | −0.7215 | −0.9485 |
| MAPK8IP2 | 1.0105 | 1.0845 | 2.6895 | −0.6035 | 0.9305 | 0.0865 |
| MARCO | 1.024 | −1.68 | −0.281 | −1.037 | −0.504 | 0.267 |
| MARLIN1 | −0.684 | 0.632 | 0.559 | 0.392 | 0.279 | −0.077 |
| MASS1 | −0.5355 | 0.9525 | 0.4675 | 0.3585 | 0.0795 | −0.1495 |
| MCM2 | 2.5715 | −1.5155 | −1.2515 | −0.6905 | −0.7945 | 1.4705 |
| MCM3 | 1.037 | 0.098 | −0.869 | 0.011 | 1.354 | 0.711 |
| MESP1 | −1.3665 | −0.2385 | −1.7255 | −1.6145 | −0.6875 | 0.6715 |
| METRN | −0.5485 | −0.6855 | −0.4135 | −0.1095 | 0.0355 | 0.0345 |
| MFAP2 | −1.048 | −0.113 | −0.47 | −0.231 | 0.134 | 0.284 |
| MFGE8 | 0.0115 | 0.1225 | 0.2435 | 0.1695 | 0.4355 | 0.6405 |
| MGAT4B | 0.72 | 0.046 | 0.973 | 0.101 | 0.868 | −0.075 |
| MGC1091 [symbol] | 0.3515 | 0.1815 | −0.4445 | 1.8925 | −0.4625 | −0.1975 |
| MGC1098 [symbol] | −0.5555 | 0.5965 | −0.0085 | 1.3265 | −0.0585 | −0.6075 |
| MGC1124 [symbol] | −0.337 | 0.443 | −0.094 | −0.314 | −0.146 | 1.074 |
| MGC1127 [symbol] | −0.352 | −0.029 | −0.108 | 0.129 | 0.331 | 0.142 |
| MGC1312 [symbol] | 0.417 | −0.149 | 0.168 | 0.423 | −0.043 | 0.085 |
| MGC1318 [symbol] | 0.478 | −0.393 | 0.46 | −0.103 | −0.207 | 0.061 |
| MGC1414 [symbol] | 0.7805 | 0.0955 | 0.3205 | −0.4565 | 0.1725 | −0.1195 |
| MGG1428 [symbol] | 0.306 | 0.041 | 0.538 | 0.824 | 0.102 | −0.009 |
| MGC1481 [symbol] | −0.046 | 0.68 | 0.179 | 1.451 | −0.082 | −0.646 |
| MGC1580 [symbol] | 0.2665 | −0.3285 | −0.1635 | −0.3555 | −0.1425 | 0.0955 |
| MGC1663 [symbol] | −0.269 | 0.503 | 0.343 | 0.448 | −0.165 | −0.568 |
| MGC1873 [symbol] | −0.748 | 0.445 | −0.588 | −0.472 | −0.49 | −0.145 |
| MGC2053 [symbol] | 1.37 | 0.332 | 0.772 | 0.688 | 0.134 | −0.237 |
| MGC2398 [symbol] | 1.471 | 0.858 | 0.497 | −0.04 | 0.872 | 0.823 |
| MGC2610 | 1.093 | 1.602 | 0.824 | −0.098 | 2.021 | −0.379 |
| MGC2714 | 1.307 | 1.776 | 0.404 | −0.155 | 1.375 | −0.669 |
| MGC2716 [symbol] | 1.3375 | 1.8415 | 0.2385 | −0.2145 | 1.5195 | −0.6045 |
| MGC2716 [symbol] | 2.1475 | 0.9295 | 0.1125 | 0.0125 | 1.8915 | −0.4865 |
| MGC2716 [symbol] | 1.092 | 2.088 | 0.053 | 0.262 | 1.845 | −0.536 |
| MGC2716 [symbol] | 1.2145 | −0.5285 | −0.1745 | 0.3275 | −0.1725 | −0.3845 |
| MGC2716 [symbol] | −0.948 | 1.383 | −0.343 | 0.433 | 0.623 | −0.342 |
| MGC2887 [symbol] | −0.384 | 0.838 | 0.039 | −0.188 | 0.68 | 0.13 |
| MGC3212 [symbol] | −0.293 | −0.529 | −0.159 | 0.504 | −0.264 | −0.454 |
| MGC3321 [symbol] | −0.6185 | 0.2065 | −0.1195 | 0.2305 | −0.3655 | −0.5385 |
| MGC3484 [symbol] | −0.0265 | 1.1895 | −0.3425 | 0.0545 | 0.2265 | 0.3755 |
| MGC3492 [symbol] | −0.594 | −0.307 | −0.901 | 1.003 | −0.229 | −0.686 |
| MGC4021 [symbol] | −0.624 | −0.049 | −0.314 | 0.295 | −0.391 | −0.668 |
| MGC4251 | 0.0515 | 0.5665 | 0.2195 | 0.6625 | 0.6965 | 0.1405 |
| MGC4308 | −0.122 | −0.584 | −0.256 | −0.856 | 0.334 | 0.255 |
| MGC4800 | −2.1875 | −1.6285 | −1.6855 | 0.6545 | −0.6065 | −0.9225 |
| MGC4659 | −0.872 | −0.519 | −0.573 | 0.181 | −0.691 | −0.765 |
| MIA | −0.7175 | −0.3235 | −0.8055 | 0.3725 | −0.0105 | −0.6445 |
| MID1 | −1.1495 | −0.6915 | −0.7975 | 0.6245 | −0.4465 | −0.4735 |
| MID1 | −0.133 | 2.059 | 0.487 | 0.992 | 0.112 | −0.01 |
| MID1 | 1.2745 | −0.1385 | 0.5455 | −0.3315 | 0.1895 | −0.3845 |
| MLF1IP | −1.5975 | −0.4055 | −1.1685 | −1.0115 | −0.8375 | 0.9615 |
| MLSTD1 | 0.082 | −0.576 | −3.774 | −2.061 | −2.506 | −1.01 |
| MMP1 | 1.247 | 0.754 | −0.138 | 0.849 | 0.068 | −0.074 |
| MMP7 | 0.0315 | −0.0205 | 0.0645 | −0.2035 | −0.0865 | −0.0405 |
| MNAT1 | 0.2835 | −0.3295 | −0.2125 | 0.4695 | −0.5555 | −0.2435 |
| MRPL14 | −0.395 | 0.406 | 0.299 | 0.48 | 0.292 | −0.341 |
| MRPL15 | 0.5485 | 0.0845 | 0.6325 | −0.0725 | 0.0745 | −0.5055 |
| MRPL27 | −0.1905 | 0.6415 | 0.0725 | 0.4605 | −0.3445 | −0.1215 |
| MRPL34 | −0.659 | −0.141 | −0.612 | 0.885 | −1.061 | −0.838 |
| MRPL43 | 0.2725 | 0.0125 | −0.2845 | 0.0075 | −0.1935 | −0.4295 |
| MRPL45 | 0.114 | −0.193 | 0.356 | 0.11 | 0.153 | −0.097 |
| MRPL48 | 0.6215 | −0.2295 | 0.5705 | 0.0545 | −0.2305 | 0.2125 |
| MRPL9 | 0.2695 | 0.2705 | 0.5925 | 0.4125 | −0.3445 | −0.0365 |
| MRPS14 | 0.385 | 0.04 | 0.333 | −0.323 | −0.225 | −0.285 |
| MRPS15 | 0.036 | 0.124 | 0.683 | −0.309 | −0.541 | 0.048 |
| MRPS17 | −0.547 | 0.086 | 0.204 | −0.282 | −0.759 | −0.245 |
| MRPS24 | 0.0455 | 0.2125 | −0.0075 | −0.0775 | 0.1925 | −0.1035 |
| MRPS25 | −0.869 | −0.119 | −0.405 | 0.244 | −0.429 | −0.048 |
| MRPS31 | 3.103 | 0.257 | 1.838 | 1.844 | 0.666 | 0.287 |
| MRPS6 | 0.1125 | 0.9155 | 0.7385 | 0.8185 | −0.2555 | 0.0035 |
| MS4A1 | 0.6945 | 0.1695 | 0.0195 | 0.6505 | 1.0515 | 0.0825 |
| MSH2 | 0.854 | −1.258 | −0.978 | 0.374 | −0.408 | 0.377 |
| MSH3 | 0.317 | 0.194 | −1.05 | 0.365 | 0.75 | 0.114 |
| MSMB | 0.706 | 0.803 | 0.286 | 0.867 | 0.221 | 0.312 |
| MTA1 | 1.5075 | 1.1005 | 0.3425 | −0.1045 | −0.0705 | −0.2795 |
| MTA3 | 0.085 | 0.059 | 0.077 | 0.103 | −0.009 | −0.121 |
| MTAC2D1 | −0.157 | 0.188 | −0.126 | 0.177 | 0.008 | 0.089 |
| MTCH1 | 0.7735 | −1.3385 | −0.2355 | −0.2975 | −1.1485 | −0.1095 |
| MTHFD1 | 1.2425 | 9.2476 | 0.6245 | 0.9585 | −0.1005 | 1.0385 |
| MTMR2 | 1.3285 | −0.2965 | −0.9205 | −0.2335 | −0.4115 | −0.2595 |
| MUC1 | 0.339 | −0.559 | −0.938 | −0.156 | 0.836 | 0.066 |
| MUC20 | −0.789 | −1.776 | −1.533 | −0.373 | −0.516 | −0.151 |
| MUM1 | 0.124 | 2.076 | 0.873 | 2.491 | 1.167 | 0.164 |
| MXI1 | −0.301 | −1.25 | 0.500 | 0.268 | −0.052 | −0.383 |
| MYB | −0.876 | 0.154 | 0.78 | 0.147 | −0.058 | −0.205 |
| MYBL1 | 0.947 | −0.099 | −0.49 | 0.32 | −0.819 | −0.163 |
| MYBL2 | 0.127 | 0.765 | 1.14 | −0.028 | −0.416 | −0.417 |
| MYO10 | 0.5145 | −0.3065 | −0.1005 | −0.2345 | 0.2795 | −0.2595 |
| MYO6 | −1.9485 | 0.7415 | −0.2885 | −1.6445 | −1.5495 | 0.6795 |
| MYST3 | −0.1255 | −0.0085 | −0.0325 | −0.1105 | 0.2555 | −0.1915 |
| NALP2 | 2.1255 | 0.9235 | 1.3935 | 0.9745 | −0.0015 | 0.1645 |
| NALP6 | −1.1525 | 0.2675 | −0.3085 | −0.2785 | 0.2395 | −0.6695 |
| NANS | 1.865 | 2.968 | 2.333 | 2.497 | 0.541 | 1.304 |
| NARF | −1.555 | −1.333 | −1.236 | 0.718 | −0.349 | −0.234 |
| NAT2 | 0.1855 | 0.4765 | 0.4555 | 0.2155 | 0.6655 | −0.3665 |
| NAV2 | 0.45 | 0.912 | 1.591 | 0.744 | −0.973 | −0.4 |
| NDE1 | 0.8325 | −1.1145 | −0.8735 | −0.0065 | 0.1695 | −0.1795 |
| NDP52 | 0.391 | 0.23 | 0.24 | 0.582 | −0.227 | −0.533 |
| NDRG2 | 0.949 | 0.397 | 0.006 | −0.471 | 0.088 | −0.051 |
| N0UFA7 | −0.0285 | −0.0165 | −0.0745 | −0.2485 | −0.2355 | −0.1615 |
| NDUFB1 | 0.979 | 0.044 | 0.717 | −0.202 | 0.151 | −0.28 |
| NDUFB10 | 0.398 | 0.075 | 0.977 | 0.288 | −0.494 | −0.289 |
| NDUFB2 | 0.8885 | 0.2405 | 0.5875 | 0.5555 | −0.4045 | 0.0685 |
| NDUFB5 | 0.5655 | 0.6145 | 0.0365 | −0.1105 | 0.2975 | 0.2705 |
| NDUFB6 | −0.4135 | −0.1565 | 0.0765 | −0.2715 | −0.3535 | −0.3135 |
| NDUFC2 | 0.2605 | 0.7155 | 1.0235 | −0.2005 | 0.6015 | −0.1095 |
| NEBL | 0.048 | −0.167 | −0.19 | −0.181 | 0.2 | −0.097 |
| NEK2 | 0.794 | 2.91 | 1.412 | 2.419 | 2.506 | 2.471 |
| NEK6 | −1.5365 | −0.4315 | 0.3505 | 1.2455 | −0.3245 | 0.3085 |
| NELL2 | 0.3435 | −0.0925 | 1.8145 | −0.2955 | −0.1395 | −0.5215 |
| NEURL | 1.651 | 0.38 | 0.266 | 1.077 | −0.339 | 0.802 |
| NFE2L3 | −0.5645 | −1.0635 | 0.3035 | −0.5015 | −0.5205 | −0.5005 |
| NFIA | 0.162 | −0.125 | 0.182 | −0.578 | 0.48 | 0.329 |
| NFIB | 0.4415 | −0.0245 | 0.9005 | 0.3935 | −0.4225 | 0.0025 |
| NINJ1 | −0.1625 | −0.1535 | −0.1785 | −0.2295 | −0.1455 | 0.0335 |
| NIPSNAP [symbol] | −0.5525 | −0.0955 | −0.1815 | −0.4995 | −0.3925 | −0.4825 |
| NKX8-2 | −0.5005 | −0.3255 | 0.1035 | −0.5325 | −0.7825 | −0.7565 |
| NME1 | 0.13 | 0.194 | 0.073 | 0.075 | 0.594 | −0.129 |
| NME2 | 1.6355 | 1.7575 | 3.0645 | 1.3805 | 0.9465 | −1.3226 |
| NME3 | 1.2575 | 0.3565 | 1.6705 | 0.2205 | 0.0915 | 0.0205 |
| NMES1 | −0.558 | 0.213 | −0.219 | −0.722 | −0.137 | 0.218 |
| NMI | −0.2055 | 0.1205 | 0.0035 | 0.5965 | −0.0595 | −0.2655 |
| NOC4 | 1.5635 | 1.8685 | 0.6585 | 0.0825 | 1.6385 | −0.2025 |
| N0LC1 | 0.0025 | −0.0735 | 0.1285 | −0.8765 | −0.4315 | −0.3675 |
| NotFound | 0.2675 | −0.0415 | 0.1665 | 0.4105 | 0.2895 | 0.2575 |
| NotFound | 0.58 | 0.509 | 0.235 | 0.11 | 0.993 | −0.372 |
| NotFound | 0.877 | 1.114 | 0.918 | 0.758 | −0.283 | −1.261 |
| NotFound | 2.874 | 0.921 | 1.548 | 0.787 | 0.703 | 0.013 |
| NotFound | 1.0575 | −0.2455 | 2.0055 | −0.2415 | 1.2885 | −0.2735 |
| NotFound | 0.222 | −0.254 | −1.005 | −0.571 | −0.333 | −0.611 |
| NotFound | 1.1005 | −1.2535 | 1.9085 | 0.3845 | −0.4635 | 1.9685 |
| NotFound | 0.604 | 0.365 | 0.734 | 0.418 | −0.191 | −0.408 |
| NotFound | −0.6595 | −0.4125 | −0.7095 | −0.1495 | 0.3575 | 0.6925 |
| NotFound | 0.8845 | 0.8185 | 0.4335 | 0.5725 | 0.9595 | −0.7175 |
| NotFound | −0.1015 | −0.0035 | 0.1715 | −0.0205 | 0.4765 | 0.1785 |
| NotFound | −0.0765 | −0.1745 | 0.0445 | −0.0275 | −0.0365 | −0.0195 |
| NotFound | 0.828 | 0.974 | 0.299 | 2.557 | 1.54 | 0.336 |
| NotFound | −0.47 | 0.476 | −0.209 | 0.198 | 0.588 | 0.159 |
| NotFound | 0.38 | 0.549 | 0.11 | 1.16 | 0.213 | 0.121 |
| NotFound | 1.1935 | 1.1925 | −0.5776 | 1.5765 | 1.2035 | 0.3225 |
| NotFound | −0.929 | 1.541 | −0.335 | 1.204 | −0.461 | −1.063 |
| NotFound | 0.326 | −0.442 | −0.225 | 0.208 | −0.04 | −0.753 |
| NotFound | 0.1005 | 0.4365 | −0.3745 | −0.4176 | 0.2495 | 0.4925 |
| NotFound | −0.3305 | −0.5435 | 0.2565 | 0.3415 | 0.4405 | 1.3185 |
| NotFound | 0.127 | −0.04 | −0.047 | −0.134 | 0.035 | −0.219 |
| NotFound | −1.1305 | −0.5135 | −0.3835 | −0.1195 | −0.5995 | 0.3265 |
| NotFound | 2.817 | 3.787 | 3.177 | 3.668 | 2.288 | 2.894 |
| NotFound | 1.031 | −1.08 | 0.911 | 0.811 | 0.331 | 1.469 |
| NotFound | −0.4355 | −0.4875 | 0.0525 | −0.3245 | −0.6405 | −0.5285 |
| NotFound | 0.438 | −0.712 | 0 | 0.239 | −0.509 | −0.068 |
| NotFound | 1.1685 | −1.1345 | 0.7715 | 0.7645 | −0.1925 | 1.4895 |
| NotFound | −0.664 | −0.417 | −0.347 | −0.099 | 0.544 | 0.211 |
| NotFound | 0.855 | 0.779 | 0.334 | 0.423 | −0.199 | −0.071 |
| NotFound | 1.1035 | 2.4805 | 0.5045 | 0.2615 | 1.4545 | −0.6115 |
| NotFound | 0.8435 | −1.3415 | 0.0605 | −0.0095 | −0.1145 | 1.4145 |
| NotFound | 0.8615 | −0.1075 | 1.2295 | 0.5705 | 0.6555 | 1.9515 |
| NotFound | 1.4585 | −0.5835 | 0.9735 | 1.0845 | 0.2075 | 1.5525 |
| NotFound | 2.1615 | 0.1725 | −1.5916 | −1.3255 | −0.4825 | 0.5875 |
| NotFound | 0.689 | −1.204 | 0.42 | −0.132 | −0.014 | 1.06 |
| NotFound | 1.2365 | −1.8025 | 0.8975 | 1.7755 | 0.6915 | 0.7705 |
| NotFound | −0.5015 | 0.3365 | −0.0535 | −0.4125 | 0.1605 | −0.1035 |
| NotFound | 1.8195 | −1.2015 | 0.4615 | −0.5825 | 0.7735 | 1.3065 |
| NotFound | −1.0715 | −1.0035 | −0.6675 | 0.0325 | −0.7015 | 0.3105 |
| NotFound | −0.488 | 0.867 | 0.877 | 0.845 | −0.625 | 0.227 |
| NotFound | 0.145 | −0.037 | 0.123 | −0.07 | −0.208 | −0.464 |
| NotFound | 0.994 | 2.109 | 0.25 | 0.187 | 0.832 | −0.317 |
| NotFound | 1.1385 | −1.0995 | 0.7375 | 0.7135 | 0.2895 | 1.4125 |
| NotFound | 0.9825 | −0.2435 | 0.1305 | 0.7785 | −0.7125 | −0.3455 |
| NotFound | −0.318 | −0.187 | −0.061 | −0.375 | 0.352 | −0.113 |
| NotFound | −0.2985 | 0.1155 | 0.1115 | −0.0615 | 0.0125 | −0.0695 |
| NotFound | 0.7555 | −0.9285 | 0.5355 | −0.5315 | −0.0275 | 0.3055 |
| NotFound | −0.6225 | −0.7055 | −0.8775 | −1.4615 | 0.2635 | −0.1595 |
| NotFound | 1.3755 | −0.2995 | 0.4185 | −0.1575 | 0.5175 | 0.3275 |
| NotFound | 0.032 | −9.083 | 0.277 | −0.537 | 0.084 | 0.026 |
| NotFound | 1.021 | 0.422 | −0.827 | 0.585 | 0.228 | −0.039 |
| NotFound | 0.9525 | 0.9855 | 1.0085 | 0.6025 | −0.2095 | −0.6995 |
| NotFound | 0.6855 | 0.0965 | 0.3795 | −0.3845 | 0.5745 | −0.7335 |
| NotFound | 0.7725 | 0.7585 | 0.8215 | 1.1955 | 0.6675 | 1.2015 |
| NotFound | −0.6375 | −0.5415 | 0.2695 | −0.4285 | 0.0385 | −0.0085 |
| NotFound | 1.586 | 1.524 | −2.548 | −1.693 | −1.699 | −0.504 |
| NotFound | 2.06 | −0.563 | −0.785 | −0.01 | −0.522 | 0.233 |
| NotFound | 0.2316 | 0.0745 | 0.0685 | −0.0905 | −0.0925 | −0.2025 |
| NotFound | 0.935 | 0.968 | −0.224 | −0.599 | 0.116 | 0.383 |
| NotFound | −0.888 | 0.293 | 0.083 | 0.076 | 0.3 | −0.592 |
| NotFound | −0.7595 | 0.2605 | −0.1165 | 1.9995 | 1.8435 | −0.1685 |
| NotFound | 0.102 | −0.1 | 0.13 | −0.111 | 0.045 | −0.245 |
| NotFound | 1.248 | −0.012 | 1.368 | 0.759 | 0.35 | 2.166 |
| NotFound | 1.6645 | 1.1925 | 0.7205 | −0.7765 | 0.2065 | −0.5415 |
| NotFound | 0.98 | 0.446 | 1.572 | 0.165 | 1.38 | 0.45 |
| NotFound | −0.7525 | 0.6015 | −0.4395 | −0.1375 | 0.5575 | −0.6305 |
| NotFound | −0.4895 | −0.3085 | −0.0185 | 0.0125 | −0.1675 | 0.0345 |
| NotFound | 1.024 | −1.176 | 0.545 | 0.471 | 0.328 | 1.418 |
| NotFound | 0.3725 | −0.0955 | 0.3735 | 0.3405 | 0.0835 | 0.1325 |
| NotFound | 0.419 | 0.364 | −0.836 | 0.883 | 0.349 | 0.076 |
| NotFound | −0.484 | 0.03 | −0.474 | −0.251 | −0.341 | −0.543 |
| NPDC1 | −0.2445 | 0.1445 | −0.4205 | 0.1365 | 0.2215 | 0.1655 |
| NPEPPS | 0.81 | −0.612 | 0.697 | −0.114 | 0.46 | −0.148 |
| NPY | 0.202 | 0.312 | 0.524 | 0.454 | −0.162 | 0.436 |
| NQO3A2 | −0.011 | 0.445 | −0.665 | 0.482 | −0.195 | 1.172 |
| NRAS | 0.030 | 0.906 | 1.4 | 0.452 | −1.146 | 0.617 |
| NRIP1 | 1.4465 | −0.5935 | 0.7395 | 0.3205 | 0.9755 | −0.3325 |
| NTN4 | 0.4735 | 0.1365 | 0.3625 | 0.2765 | −0.1865 | 0.1315 |
| NUCB2 | −0.2755 | 1.0095 | 0.0295 | 0.3075 | 0.2265 | 0.3355 |
| NUDT5 | −0.134 | 0.170 | −0.454 | 0.444 | −0.282 | −0.376 |
| NUDT6 | 0.027 | −0.054 | 0.838 | 0.045 | 0.323 | 0.206 |
| OCIAD2 | 0.4945 | −0.1085 | −0.2885 | −0.2725 | −0.8225 | −0.9315 |
| OFD1 | −0.5665 | −2.4045 | −0.3995 | −0.6945 | −0.6195 | 0.1325 |
| OGFRL1 | −0.539 | −0.047 | −0.146 | 0.062 | −0.103 | 0.109 |
| OPLAH | −0.3855 | 0.1985 | −0.0655 | −1.2595 | 0.7735 | −0.1875 |
| ORMDL3 | −1.618 | 0.291 | 1.268 | 0.439 | 0.685 | 1.927 |
| OSBPL3 | 0.659 | −0.335 | 0.501 | 0.066 | 0.132 | 0.242 |
| OSR2 | 0.7075 | 0.8445 | −0.0265 | 0.1605 | 0.5625 | −0.5405 |
| OSTF1 | 1.312 | 0.419 | −0.339 | −0.942 | −0.041 | 0.123 |
| P2RX1 | 1.1815 | 1.7375 | 1.3405 | 0.7345 | 1.8535 | −0.5555 |
| P2RY2 | −0.107 | −0.99 | −0.637 | −0.344 | −0.35 | −0.279 |
| PACAP | −0.002 | 0.178 | 0.283 | −0.195 | −0.269 | −0.034 |
| PADI2 | 1.0335 | −0.8305 | 0.2885 | −1.5585 | −1.2125 | −0.1435 |
| PAPD5 | 0.149 | 0.591 | −0.076 | −0.392 | −0.22 | −0.123 |
| PAPSS2 | 0.573 | −0.489 | 0.477 | 0.412 | 0.091 | −0.253 |
| PARD8A | 1.137 | 1.362 | 1.402 | −0.012 | 0.053 | −0.001 |
| PARP1 | 0.037 | 3.007 | −0.049 | 0.634 | 0.541 | −0.004 |
| PARP9 | 0.9625 | −0.4835 | 0.9095 | −0.8985 | −0.3575 | 0.1485 |
| PAXIP1L | −0.2375 | −0.5845 | −0.3995 | −0.3495 | 0.6425 | 0.1715 |
| PBX3 | −1.1575 | −0.6375 | 0.3795 | −0.2515 | 0.3315 | 0.4335 |
| PCDHB11 | 0.384 | −0.971 | −1.274 | 0.815 | 1.842 | 0.545 |
| PCDHB16 | 1.033 | 2.108 | 0.615 | 1.028 | 0.154 | 0.754 |
| PCDHB2 | 0.1675 | 0.9045 | 1.3855 | 0.2885 | −0.1595 | −0.2405 |
| PCM1 | 0.6405 | 0.5845 | 1.6285 | −0.0635 | 1.1635 | −0.1195 |
| PCNA | −0.032 | −0.309 | 0.616 | 0.221 | −0.193 | 0.241 |
| PDC01 | 1.0205 | 0.4495 | −0.8535 | 0.5105 | 1.5395 | 0.3715 |
| PDC010 | 0.169 | −0.074 | 0.205 | 0.154 | −0.504 | −0.203 |
| PDCD4 | 0.7185 | 0.2705 | 0.6195 | 0.4675 | −0.1575 | −0.3005 |
| PDCD5 | −0.1715 | −0.1965 | 0.8385 | −0.2745 | 0.9785 | −0.0605 |
| PDCD8 | 0.0285 | −0.1025 | 0.3845 | 0.2385 | −0.2415 | −0.1885 |
| PDE4DIP | 0.1565 | 0.4045 | 1.5735 | 0.4615 | 0.0235 | −0.0415 |
| PDHA1 | −1.941 | −1.581 | 2.042 | −1.421 | 2.345 | −0.551 |
| PECI | 0.1855 | −0.0545 | 0.1355 | 0.0055 | −1.0315 | −0.0055 |
| PEG10 | 0.5355 | 0.2265 | 0.0795 | 0.8495 | −0.6025 | −1.3625 |
| PER2 | 0.426 | 0.524 | −0.071 | −0.32 | −0.006 | −0.594 |
| PERP | −0.1225 | 0.6395 | −0.1425 | 0.1595 | 0.2375 | 0.2575 |
| PEX1 | 0.631 | −0.382 | −0.087 | 0.03 | −0.294 | 0.019 |
| PFAAP5 | −0.4925 | −1.0745 | 0.3365 | −0.7185 | −0.8005 | 1.2605 |
| PFDN2 | −0.273 | 0.505 | −0.132 | 0.518 | 0.297 | 0.114 |
| PGM1 | 0.4785 | 0.3735 | 0.3325 | 0.6195 | −0.0555 | 0.2025 |
| PGR1 | 0.4615 | 1.4405 | 1.0175 | 0.0095 | −0.8315 | −0.4525 |
| PHAX | −0.387 | 0.356 | −0.526 | −0.305 | −0.538 | −0.703 |
| PHGDH | 0.6395 | 0.4905 | 0.7205 | −0.3835 | −0.5005 | −1.3985 |
| PHYH | 0.5705 | 0.1935 | 0.8265 | 0.3845 | 0.2245 | 0.3105 |
| PI85PA | 0.7685 | 1.3625 | 0.5465 | 0.1885 | 0.9445 | −0.4915 |
| PIGT | −0.142 | 0.225 | −0.313 | −0.485 | 0.088 | −0.506 |
| PIM2 | −0.6935 | 0.2525 | 0.2875 | −0.8555 | 0.5225 | −0.3105 |
| PISD | 1.533 | 2.655 | 1.904 | 4.093 | 1.117 | −0.015 |
| PITPNC1 | −0.4085 | 0.0775 | 0.7275 | 0.0015 | −0.1885 | −0.2175 |
| PKIB | −0.171 | 0.702 | −0.299 | −0.303 | −0.06 | 0.547 |
| PKMYT1 | 2.374 | −0.659 | 2.6 | 1.105 | 1.214 | 0.313 |
| PLA2G4B | 1.0365 | −0.8005 | −0.0045 | −0.0295 | 0.4005 | 0.3995 |
| PLAC8 | −0.334 | 0.745 | 0.19 | 0.04 | 0.053 | −0.077 |
| PLCB1 | 0.3665 | 1.2745 | −0.4655 | 1.0555 | 0.0845 | 0.1935 |
| PLCXD1 | −1.326 | −0.275 | −0.744 | −0.796 | −0.241 | 0.255 |
| PLEK2 | −0.2135 | −0.3235 | −0.0445 | −0.0435 | −0.2325 | 0.1665 |
| PLO0 | 0.9735 | −0.0595 | 0.7335 | 0.0555 | −0.3195 | 0.0125 |
| PNAS-4 | −0.4745 | −0.2395 | −0.1965 | −0.3885 | 0.4765 | −0.0385 |
| PNLIPRP2 | −0.0465 | 0.3245 | −0.1655 | 0.3425 | 0.5035 | −0.4215 |
| PNMA3 | −0.5365 | −0.1775 | −0.4745 | −0.8955 | −0.4395 | −0.3175 |
| POLB | −0.242 | 0.183 | −0.381 | 0.282 | −0.006 | −0.1 |
| POLR2F | 0.0705 | −0.2985 | 0.4905 | −0.5165 | −0.3055 | −0.7405 |
| POLR2H | 0.5515 | 0.1005 | 0.4505 | 0.1565 | 0.1235 | −0.0175 |
| POMC | 0.134 | −0.151 | 0.318 | 0.199 | −0.517 | −0.435 |
| POP4 | −0.176 | −0.136 | −0.364 | −1.005 | −0.688 | 0.094 |
| POP7 | −0.46 | 0.091 | −0.679 | −0.227 | −0.68 | 0.203 |
| PP1057 | −0.0905 | 0.0635 | 0.0685 | 0.3585 | −0.4195 | −0.2935 |
| PPFIA1 | −0.016 | −0.282 | −0.003 | −0.155 | −0.159 | −0.357 |
| PPFIBP2 | 0.2345 | 0.2165 | 0.0055 | 0.6465 | 0.3695 | 0.2415 |
| PPGB | 0.7165 | 0.0015 | 0.4725 | 0.5365 | −0.0845 | −0.3915 |
| PPHLN1 | 0.3585 | −0.1255 | 0.3605 | −0.1285 | −0.2825 | −0.4715 |
| PPIF | −0.3285 | 0.4055 | 0.4845 | −0.1495 | 0.3995 | 0.0035 |
| PPIL1 | −0.1225 | 1.0575 | −1.4785 | 0.2575 | 0.7015 | 0.4605 |
| PPOX | 1.028 | 2.523 | 3.429 | 0.02 | 2.806 | 0.33 |
| PPP1R16 [symbol] | −0.377 | 0.526 | −0.157 | 0.158 | 0.758 | 0.214 |
| PPP1R3C | 0.967 | −0.218 | 0.703 | 0.716 | 0.149 | 0.394 |
| PPP2R2D | −0.8395 | −0.5995 | −0.5265 | −0.0455 | −0.4755 | 0.1165 |
| PPP2R5A | −0.43 | 0.06 | 1.108 | −0.357 | −0.034 | −0.315 |
| PRAME | −0.3175 | 0.0655 | 0.0735 | 0.4565 | −0.0015 | 0.0495 |
| PRC1 | 1.11 | −0.163 | 0.655 | 0.043 | 0.048 | 0.621 |
| PRIM2A | 1.6485 | 0.0295 | 2.7165 | 1.2665 | 1.6255 | 0.0045 |
| PRKACB | −0.518 | −0.114 | 0.413 | −0.051 | −0.514 | −0.502 |
| PRKCB1 | 0.06 | −0.083 | 0.026 | −0.548 | 0.067 | 0.135 |
| PRKCI | 0.328 | −0.237 | −0.297 | −0.492 | 0.585 | −0.295 |
| PRKRIR | 0.1935 | −0.7965 | 0.5365 | −0.3045 | 0.3935 | −0.5425 |
| PRKWNK1 | −0.8825 | −1.1975 | −0.5175 | −0.3035 | −1.0735 | −0.8435 |
| PRKX | 0.331 | 0.958 | 0.183 | 0.186 | −0.058 | −0.245 |
| PRNP | 0.3445 | −0.2735 | 0.1955 | 0.0605 | −0.2835 | −0.3625 |
| PROL5 | −1.0425 | 0.2035 | −0.2245 | −0.4535 | −0.0365 | −0.7255 |
| PRPF18 | −0.1435 | −0.3425 | −0.1035 | 0.3105 | 0.3955 | 0.1785 |
| PRPSAP1 | 0.4075 | 0.1285 | 1.2155 | 1.2976 | −1.1165 | −1.5335 |
| PRSS12 | 0.4735 | 1.1295 | 1.9725 | 1.1515 | 1.1945 | 2.0845 |
| PRSS16 | 0.9575 | 0.1745 | 0.4485 | 0.0835 | 0.3195 | −0.1265 |
| PSD3 | −0.3555 | −0.1275 | 0.3185 | −0.3945 | 0.1915 | −0.4585 |
| PSMA3 | −0.189 | 0.524 | −0.053 | 0.818 | 1.048 | −0.097 |
| PSMA7 | 0.371 | 0.239 | 0.044 | 0.465 | −0.398 | 0.124 |
| PSMC5 | −0.4355 | −0.0865 | 0.1715 | −0.3905 | −0.0115 | −0.8655 |
| PSMC6 | −0.362 | 0.206 | 0.677 | −0.399 | −0.053 | −0.167 |
| PSMD12 | −0.6605 | −0.1495 | −0.5635 | 0.5075 | −0.5715 | −0.3905 |
| PSMD12 | 1.7075 | 2.2915 | 0.3115 | −1.4495 | −1.3945 | −0.0695 |
| PSMD3 | 0.4055 | 0.8145 | −0.5155 | −0.4155 | −0.2995 | −0.2555 |
| PSPH | −0.223 | 0.051 | −0.339 | −0.042 | −0.62 | 0.417 |
| PTD012 | −0.399 | −0.198 | −0.66 | −0.247 | 0.625 | −0.21 |
| PTGER3 | 1.31 | −0.678 | −0.056 | 0.022 | 0.481 | 0.032 |
| PTHLH | 0.0015 | 0.1755 | 0.2715 | 0.5265 | −0.2925 | −0.4865 |
| PTK6 | 0.885 | 0.591 | 0.773 | −0.032 | 1.005 | 0.196 |
| PTPN4 | 0.385 | −0.051 | 0.248 | −0.559 | −0.763 | −0.166 |
| PTPN7 | 2.478 | −0.294 | 1.153 | 2.288 | −0.147 | −0.208 |
| PTPRF | −0.621 | −0.288 | −0.491 | 1.389 | 1.098 | 0.188 |
| PTPRN2 | −0.2365 | 0.4725 | 0.2745 | −0.1715 | −0.2225 | −0.2895 |
| PTPRT | −0.3055 | 0.3885 | 0.9535 | −0.1015 | 0.5635 | −0.1835 |
| PTS | 1.169 | 0.784 | 1.071 | 0.55 | −0.292 | −0.21 |
| PTTG1 | −0.6405 | 0.8105 | 1.5745 | 0.1195 | −0.5035 | 0.2005 |
| PURB | 1.3685 | 1.1576 | 1.1185 | 0.5085 | 1.0785 | 0.9115 |
| PXMP2 | −0.8965 | −0.1165 | 0.5375 | 0.6385 | 0.5745 | 0.0695 |
| PYCARD | −0.498 | 0.021 | 0.212 | −0.2 | 1.308 | 0.207 |
| PYCR1 | 0.178 | 0.214 | −0.702 | −0.641 | −0.372 | 0.01 |
| QDPR | 0.8495 | 0.7045 | −0.2675 | 1.7345 | 1.0235 | 0.0315 |
| QPRT | 1.228 | −0.051 | −0.615 | 1.019 | −1.381 | −0.45 |
| QSCN9 | 0.8315 | 0.7235 | 0.2385 | 0.5045 | 0.1385 | −0.9195 |
| RAB11FIP | 0.101 | −0.316 | −0.435 | −0.042 | 0.571 | 0.088 |
| RAB11FIP | 1.163 | −0.073 | 1.83 | 0.382 | 0.604 | 0.27 |
| RAB13 | 1.193 | −0.143 | 0.059 | −0.029 | 0.325 | 0.304 |
| RAB27A | −1.369 | −0.18 | −2.11 | 0.233 | −0.264 | −0.338 |
| RAB30 | 0.4725 | 0.3655 | 0.0635 | 0.2055 | 0.2115 | 0.0235 |
| RAB34 | −0.069 | −0.016 | 0.172 | −0.357 | 0.785 | 0.204 |
| RAB35 | −0.175 | 0.001 | −0.196 | −0.28 | 0.202 | −0.259 |
| RAB39B | 0.7405 | 0.9055 | 1.5295 | 0.1835 | 0.3955 | 0.1005 |
| RAB3A | −0.451 | 2.289 | −0.692 | 0.814 | 2.693 | −0.635 |
| RAB3IP | 1.189 | 0.002 | 0.278 | 0.065 | 0.725 | 0.045 |
| RABEP1 | 0.219 | 0.545 | 1.2 | −0.077 | 0.049 | 0.09 |
| RABGAP1 | 0.3995 | 0.7025 | 0.2205 | 0.3385 | 0.2245 | −0.1185 |
| RACGAP1 | −0.3515 | 0.0855 | 0.9365 | 0.6125 | 0.0245 | −0.1885 |
| RAD17 | 0.169 | 0.946 | 1.109 | 0.369 | −0.02 | −0.038 |
| RAD51 | −0.189 | −0.033 | −0.206 | 0.274 | −0.115 | −0.214 |
| RAD51AP | −0.1775 | 0.7555 | 0.8145 | 0.0575 | −0.0275 | −0.1535 |
| RAD52 | 0.121 | −0.034 | 1.224 | 0.444 | 0.091 | −0.589 |
| RAD54L | −0.8785 | 0.8125 | 0.3845 | −0.5225 | 0.3075 | −0.5205 |
| RAMP | 0.8505 | −0.6505 | −0.1425 | −1.4045 | −1.1805 | −1.6935 |
| RANBP1 | 0.1425 | 1.2455 | 0.4985 | −0.1385 | 0.4205 | 0.1265 |
| RARRES1 | −0.152 | −0.168 | −0.297 | 1.848 | 0.455 | 0.029 |
| RBBP7 | 0.2085 | 0.4575 | 0.5545 | 0.5825 | 0.6535 | 0.5725 |
| RBBP8 | −0.1995 | 0.4675 | 0.5645 | −0.3785 | −0.3155 | −0.1865 |
| RBM8A | 1.825 | −0.765 | −0.827 | −0.029 | −0.414 | −0.282 |
| RBX1 | −0.135 | 0.045 | 0.336 | −0.117 | 0.194 | 0.24 |
| RDHE2 | −0.023 | 0.431 | 0.843 | 0.494 | −0.131 | 0.317 |
| REA | −0.149 | −0.035 | −0.044 | 0.038 | 0.108 | 0.012 |
| REC14 | 1.1715 | −0.1965 | 0.7825 | −0.3385 | 0.0945 | −0.2895 |
| REG1A | 0.503 | 0.557 | −0.246 | 0.18 | 0.633 | 0.223 |
| RELB | 0.541 | 1.971 | 0.957 | 0.385 | 0.142 | 0.353 |
| REPIN1 | 0.821 | 1.051 | 0.792 | 0.737 | −0.116 | −0.253 |
| REPS2 | −0.1985 | 0.3265 | 0.9315 | 0.2795 | −0.5025 | −0.1015 |
| RERG | 0.2865 | −0.2485 | 1.3335 | 0.5555 | 0.3465 | −0.2815 |
| RFC4 | 1.0215 | 0.0225 | 0.8705 | 0.0045 | −0.0105 | −0.3645 |
| RGS10 | −0.2505 | 2.6305 | 1.4895 | −0.0075 | 0.0745 | −0.2185 |
| RH8DL2 | −0.255 | 0.449 | −0.342 | 0.382 | 0.38 | 0.324 |
| RIBC2 | −0.086 | 0.251 | 0.127 | −0.102 | 0.188 | 0.464 |
| RIC-8 | −0.392 | −0.099 | 0.086 | −0.484 | −0.638 | −0.553 |
| RIT1 | −0.008 | 0.258 | 0.074 | 0.051 | 0.193 | 0.139 |
| RLN2 | 0.184 | −0.012 | −0.065 | 0.284 | −1.159 | −0.307 |
| RNF10 | −0.3785 | 0.3915 | 0.2435 | 0.3655 | 0.4005 | 0.3785 |
| RNF135 | −0.0145 | −0.7395 | −0.2835 | 0.4905 | 0.5705 | 0.4085 |
| RNF7 | 0.488 | 0.431 | 1.591 | 0.386 | 0.138 | 0.767 |
| ROPN1 | −0.6275 | −0.6055 | −1.2385 | −0.0975 | 1.2235 | 0.7345 |
| RPA3 | −0.4576 | −0.2915 | −0.0945 | −0.3345 | −0.0715 | −0.2095 |
| RPESP | −1.016 | −0.92 | −0.807 | −0.221 | 0.113 | −0.098 |
| RPL11 | 0.035 | −0.376 | −0.253 | −0.098 | 0.082 | −0.383 |
| RPL13 | −0.884 | −0.484 | 0.05 | 0.496 | −0.124 | −0.35 |
| RPL13A | −0.0515 | −0.4895 | −0.0315 | 0.0775 | −0.1925 | −0.4385 |
| RPL15 | −0.361 | 0.023 | −0.129 | 0.826 | −0.554 | −0.624 |
| RPL18A | −0.151 | −0.068 | 0.047 | −0.792 | −0.027 | −0.049 |
| RPL19 | −0.596 | 0.245 | −0.048 | 0.22 | −0.68 | −0.989 |
| RPL21 | −0.3805 | 0.2305 | 0.0765 | 0.2105 | −0.8085 | −0.8245 |
| RPL23A | −0.528 | −0.374 | −0.184 | 0.379 | −0.168 | −0.424 |
| RPL23A | −0.8855 | −0.6425 | −0.0295 | −0.3035 | 0.0515 | −0.1155 |
| RPL26L1 | −0.5135 | −0.7655 | −0.4115 | −0.6915 | −0.1135 | −0.7045 |
| RPL29 | 0.2645 | −0.0515 | −0.0845 | −0.1875 | 0.1115 | 0.0995 |
| RPL35 | 0.3225 | −0.2825 | 0.3195 | 0.0405 | −0.0545 | −0.4465 |
| RPL35AL | −0.2945 | 0.0945 | 0.0075 | 0.2045 | 1.1045 | 0.7245 |
| RPS16 | −0.1555 | −0.1025 | −0.1415 | 0.2415 | −0.0315 | −0.0305 |
| RPS4Y1 | −0.1185 | −0.3075 | −0.5255 | −0.8905 | −0.1055 | −0.3445 |
| RRAGA | −0.731 | 0.85 | 0.467 | 0.628 | −0.091 | −0.148 |
| RRAGD | −0.681 | 1.329 | 2.386 | 0.515 | −0.206 | 0.105 |
| RRM1 | 0.159 | −0.126 | −0.159 | 0.172 | 0.047 | 0.048 |
| RRM2 | −0.764 | −1.524 | −0.507 | −0.493 | −0.439 | −0.349 |
| RSAFD1 | −0.6955 | −0.4985 | −0.0255 | −0.1005 | −0.3835 | −0.2625 |
| RSNL2 | 0.5295 | −0.0335 | 0.6045 | 0.1635 | −0.2385 | −0.7435 |
| RSU1 | 1.068 | −0.107 | 0.167 | 0.53 | −0.52 | 0.142 |
| RTN4IP1 | 0.007 | −0.091 | −0.69 | 0.791 | −0.092 | 0.165 |
| S100A11 | −0.436 | −0.435 | −0.657 | −0.094 | −1.019 | 0.563 |
| S100A14 | 0.9345 | −0.6905 | −1.3435 | −1.7115 | −0.7855 | −1.2725 |
| S100A16 | −0.2205 | 0.7505 | 0.7035 | 0.1016 | 0.3095 | 0.3135 |
| S100A9 | 1.255 | −0.39 | −0.878 | −0.134 | 0.478 | 0.258 |
| SAE1 | 0.6445 | 0.2715 | 1.0545 | −0.4855 | −0.3535 | 0.4915 |
| SALL2 | 1.547 | 0.814 | 1.362 | 0.427 | −0.466 | 0.317 |
| SARA1 | −0.2545 | −0.0695 | 0.0925 | 0.4235 | 0.0135 | 0.1785 |
| SARA2 | −0.549 | −0.172 | −0.085 | 1.218 | 0.016 | −0.251 |
| SAS | 0.027 | −1.088 | 1.103 | 0.818 | −0.817 | 0.215 |
| SCIN | 0.0125 | −0.4085 | 0.3885 | 0.1685 | −0.5205 | −0.6155 |
| SCML1 | −0.53 | −0.322 | −0.935 | −0.099 | −0.004 | −0.305 |
| SCPEP1 | 0.0945 | −0.3335 | −1.3815 | −0.9845 | −0.7905 | 0.5695 |
| SCRIB | −0.171 | 0.23 | −1.232 | 0.878 | −0.043 | 0.4 |
| SDC1 | 0.06 | −0.221 | 0.373 | 0.178 | 0.003 | −0.408 |
| SDC3 | 0.4355 | −0.6005 | 0.3895 | −0.3515 | −0.3845 | 0.0285 |
| SDCCAG0 | −0.7035 | 0.8595 | −0.4305 | −0.2835 | 0.1735 | 0.5355 |
| SEC61G | 0.493 | −1.864 | 0.549 | 0.809 | −0.166 | 0.053 |
| SECP43 | −1.8695 | −0.2925 | −0.8985 | 1.7815 | 1.0895 | −0.1375 |
| SEDLP | 1.935 | 0.555 | 0.063 | 0.284 | 0.721 | 0.544 |
| SEMA3B | 0.0925 | −0.2835 | −0.1985 | 0.0695 | 0.1365 | 0.1215 |
| SEMA3C | 0.1676 | 0.0735 | 1.5385 | 0.1865 | −0.3555 | −0.2635 |
| SEPW1 | 3.765 | −0.999 | −1.39 | 2.715 | 2.132 | −0.4 |
| SERP1 | −1.5545 | −0.0545 | −0.7675 | 0.7525 | −0.0005 | −0.0585 |
| SERPINA [symbol] | −0.311 | −2.074 | −1.564 | −0.146 | −0.965 | −0.113 |
| SERPINB [symbol] | −0.39 | 0.709 | 0.03 | −0.004 | 0.496 | 0.222 |
| SFRP1 | −0.795 | −0.011 | −0.593 | −0.418 | −0.234 | 0.587 |
| SFRS6 | −0.4195 | −1.2055 | −0.2525 | −0.0995 | 0.3565 | 0.2435 |
| SGCD | 1.6315 | 1.6615 | 2.3675 | 1.1935 | 1.9735 | 0.3395 |
| SGCE | 0.381 | −0.511 | −0.072 | 0.117 | 0.161 | −0.247 |
| SH3BGRL | −0.288 | 1.74 | 1.227 | 1.855 | 1.023 | −0.509 |
| SHFM1 | −0.5535 | 0.1435 | −0.2495 | −0.5355 | −0.0725 | −0.5985 |
| SIAT7B | 1.996 | 0.698 | 2.663 | 0.519 | 1.165 | 0.061 |
| SIGLEC12 | −0.102 | −0.55 | −0.107 | −0.173 | −0.432 | −0.672 |
| SIT | 0.4035 | 0.0715 | 0.1625 | −0.1425 | 0.1375 | −0.0275 |
| SITPEC | 0.3475 | −0.0955 | −0.1595 | 0.0185 | 0.2825 | −0.0575 |
| SKP1A | 0.4835 | 2.0385 | −1.1385 | 0.9165 | 0.1485 | 0.3585 |
| SLC12A9 | −0.841 | 0.447 | 0.236 | 1.953 | 1.237 | −0.958 |
| SLC13A3 | 0.5985 | 0.1075 | 0.4815 | 1.2775 | 0.2665 | 1.0085 |
| SLC16A6 | 1.528 | −1.308 | −0.789 | 2.906 | 2.247 | 2.647 |
| SLC19A2 | 1.3475 | 0.6275 | −0.6925 | −0.7755 | −0.2575 | −0.3475 |
| SLC1A1 | 1.0365 | 0.7675 | −0.7655 | 0.6135 | −0.2005 | 0.7245 |
| SLC1A2 | 0.0185 | 0.0565 | −0.7515 | 0.6945 | 0.8595 | 0.6225 |
| SLC24A3 | 0.243 | −0.637 | −1.865 | 0.128 | −0.444 | −0.341 |
| SLC25A29 | 0.94 | 0.206 | 1.154 | 0.126 | −0.06 | −0.073 |
| SLC25A4 | 1.526 | 3.158 | 0.813 | 2.785 | 1.086 | 0.112 |
| SLC25A5 | −0.8165 | −1.3155 | −0.7725 | 0.0205 | −0.8265 | −0.4275 |
| SLC27A2 | 1.055 | 1.58 | −0.125 | 0.035 | 1.26 | 0.147 |
| SLC27A5 | 0.009 | −0.985 | −0.534 | −0.517 | −0.327 | 0.089 |
| SLC30A8 | 1.7805 | 1.5715 | 1.5805 | 2.7675 | 0.5345 | 0.4185 |
| SLC39A4 | 1.163 | 1.621 | 0.094 | −1.708 | 1.361 | 1.39 |
| SLC39A6 | −1.0615 | −0.4525 | −0.5095 | −0.2975 | −0.3125 | −0.0785 |
| SLC40A1 | 0.6185 | 0.5925 | −0.0545 | −0.3955 | 0.4795 | −0.2815 |
| SLC41A3 | −0.225 | 0.703 | −0.238 | 0.006 | 0.736 | 0.043 |
| SLC5A6 | 0.2755 | 1.0675 | 0.9425 | −0.0805 | 1.2405 | −0.2255 |
| SLC6A4 | −2.7675 | 3.8125 | −2.5455 | 0.7565 | 2.5165 | 0.8445 |
| SLC9A3R1 | −0.1965 | −0.8875 | −0.1005 | −1.1955 | 0.4745 | −0.1905 |
| SLITRK6 | −0.551 | 0.168 | −0.282 | 0.306 | −0.3 | 0.192 |
| SMA3 | −1.465 | −0.364 | −1.054 | −0.643 | −0.196 | −0.029 |
| SMAD2 | 0.148 | 0.101 | 0.359 | 0.116 | 0.425 | 0.424 |
| SMO | −0.52 | 1.153 | −0.063 | −0.158 | 0.525 | −0.072 |
| SMU1 | 0.187 | −0.238 | 0.03 | 0.369 | 0.133 | 0.079 |
| SNPH | −0.097 | 0.014 | −0.547 | 0.375 | 0.478 | 0.002 |
| SNRPB2 | −0.2365 | 0.6025 | 0.5915 | −0.1815 | −0.0295 | −0.3515 |
| SNRPD1 | 0.537 | 0.172 | 0.699 | 0.193 | −0.115 | 0.11 |
| SNRPD3 | −0.6395 | 0.7425 | 0.7935 | 0.2595 | −0.7655 | −0.3985 |
| SNRPG | −1.0295 | 0.8205 | 0.6625 | −0.8085 | 0.2655 | −0.6135 |
| SNX7 | 0.1615 | 1.2965 | 0.3515 | 0.0725 | 0.0285 | −0.0755 |
| SO02 | −0.6505 | −0.6345 | −1.4345 | −0.7825 | 0.1275 | −0.1555 |
| SORCS1 | 0.459 | 0.038 | −2.945 | 0.803 | −1.05 | −0.763 |
| SOX4 | −1.5955 | −2.2685 | −0.5465 | −1.3865 | 1.4585 | 0.8455 |
| SOX9 | −0.641 | 1.038 | 0.559 | −0.317 | −0.231 | −0.882 |
| SPAG18 | 0.1105 | 0.0085 | −0.0485 | 0.5745 | 0.4345 | 0.2685 |
| SPAG5 | 0.3365 | −0.4055 | −0.6116 | −0.2985 | 0.3885 | −0.0635 |
| SPC18 | −0.923 | −0.003 | −1.09 | −0.896 | −0.15 | 0.169 |
| SPFH2 | 0.482 | −0.138 | −0.608 | 0.299 | −0.5 | −0.198 |
| SPG3A | 1.185 | −0.015 | −0.11 | 1.047 | −1.185 | −0.285 |
| SPPL3 | 0.974 | 0.522 | 1.578 | −0.254 | 0.099 | 0.146 |
| SQLE | −0.775 | 1.029 | 0.505 | 0.918 | 0.733 | −0.205 |
| SQRDL | 0.2195 | −0.0085 | −0.2565 | 0.2535 | −0.7095 | −0.4085 |
| SREBF1 | −0.0835 | 0.1815 | −0.1005 | 0.0765 | −0.0575 | −0.2615 |
| SRI | 0.0005 | −0.2975 | 1.0725 | 0.5875 | −0.3645 | −0.0955 |
| SRPK1 | 0.9235 | 0.4575 | 0.4745 | 0.0845 | 0.4345 | −0.1175 |
| SSFA2 | 0.7045 | −0.0485 | 0.4875 | −0.0005 | −0.3325 | 0.1405 |
| SSR4 | 0.7785 | −0.1295 | −0.0815 | 0.0125 | 0.3345 | −0.2855 |
| STARD3NI | 1.4865 | 1.8325 | 2.4925 | 0.2575 | 0.5935 | −0.4835 |
| STARD7 | −1.6725 | −1.7025 | −2.1985 | 0.8955 | 0.8825 | 1.9765 |
| STAT1 | −0.3115 | 0.3305 | −0.3675 | 0.0925 | 0.0175 | −0.0825 |
| STC2 | −0.1665 | 0.2825 | 0.5405 | −0.1485 | 0.5765 | −0.0995 |
| STK24 | −1.1665 | 0.8165 | 0.3955 | −0.0485 | 0.2245 | 0.0875 |
| STK6 | −0.301 | 0.092 | −0.317 | 0.089 | −0.691 | 0.307 |
| STMN1 | 0.1525 | 0.2185 | −1.0865 | 0.6255 | −0.0165 | −0.0345 |
| STX1A | 0.5055 | 0.1705 | 0.5576 | −0.1275 | 0.1235 | 0.0645 |
| STX3A | −0.1515 | 0.0025 | −0.4005 | −0.0735 | 0.3475 | −0.3055 |
| STYX | −1.1715 | −0.1995 | −0.7435 | 0.2855 | 0.0325 | 0.1155 |
| SUGT1 | −0.494 | 0.278 | 0.245 | 0.354 | 0.174 | −0.511 |
| SUMF1 | 0.2345 | −1.5075 | 0.4525 | 0.4655 | 0.2015 | 0.0325 |
| SUMO2 | 0.2165 | 0.4695 | 0.5825 | 0.3375 | 0.2915 | 0.2455 |
| SUV39H2 | 1.3795 | 0.5835 | 0.3635 | 1.9265 | −0.4315 | −1.3375 |
| SYAP1 | −1.1185 | −1.4365 | 0.1045 | −1.3055 | −0.5365 | 0.0825 |
| SYCP2 | 0.797 | 2.614 | 0.209 | −0.118 | 0.507 | 0.773 |
| SYNGR1 | 0.6475 | 0.5315 | 0.4535 | 0.0585 | −0.2295 | −0.3985 |
| SYTL2 | −0.1435 | −0.0205 | 0.0005 | −0.0105 | 0.1075 | −0.0695 |
| TAF18 | 0.6075 | 0.2565 | 0.1335 | 0.1095 | −0.0815 | −0.1355 |
| TAF8 | 0.082 | 0.035 | 1.107 | −0.257 | 0.393 | −0.485 |
| TARBP1 | 0.242 | −0.619 | −0.795 | −0.295 | −0.495 | −0.683 |
| TARS | −0.0425 | 0.6035 | 0.8755 | −0.0835 | −0.1785 | −0.1075 |
| TATDN1 | −0.5365 | −0.3705 | −0.4645 | 0.1785 | 0.2335 | 0.0115 |
| TBC1D12 | −1.9545 | −0.2725 | −0.6965 | 0.9015 | 0.2465 | −0.3975 |
| TBC1D14 | 0.418 | 0.228 | 0.26 | 0.263 | 0.04 | −0.095 |
| TBC1D3 | −1.9435 | −1.8665 | −0.9665 | −1.6535 | 0.3295 | −1.0945 |
| TBCA | −0.232 | 0.572 | 0.409 | 1.259 | −0.413 | −0.971 |
| TBX1 | −0.3965 | 0.8535 | −0.1185 | −0.1075 | −0.1735 | −0.6145 |
| TBX3 | 0.101 | −0.119 | −0.073 | 0.099 | −0.018 | −0.105 |
| TCAP | −0.022 | −0.114 | −0.321 | 0.707 | −0.255 | 0.149 |
| TCEAL1 | −0.1395 | 0.3855 | −0.6505 | 0.9465 | 0.0945 | 0.2825 |
| TCEAL3 | 0.521 | −1.012 | −0.664 | 1.426 | 0.316 | −0.957 |
| TCEAL4 | −0.424 | 0.178 | −0.455 | −0.424 | 0.036 | 0.011 |
| TCF15 | −0.698 | −0.206 | −1.387 | 0.699 | −0.549 | 0.213 |
| TCF3 | 1.103 | 0.824 | 0.54 | 0.575 | 0.134 | 0.207 |
| TCF7L2 | 0.3705 | 0.7405 | 0.0295 | 0.7815 | −0.0175 | 0.2725 |
| TDRD7 | 0.8955 | −0.0235 | −3.8575 | 0.1325 | 0.4565 | 0.2216 |
| TESK2 | −0.278 | −1.14 | −1.707 | 0.176 | −0.333 | −1.601 |
| TFAP2B | −0.248 | 1.048 | 0.47 | 1.078 | −0.679 | −0.326 |
| TFCP2L1 | 2.9755 | 1.5005 | −0.4535 | 4.8015 | 2.7455 | 1.8025 |
| TFCP2L2 | 3.0735 | −0.2905 | −1.5415 | 2.8295 | 2.0655 | 1.1655 |
| TFF1 | 0.0045 | 0.7405 | 0.8525 | −0.6885 | −0.8745 | −0.0335 |
| TFF3 | −0.001 | −0.235 | 0.19 | 1.96 | −0.248 | −0.255 |
| TFRC | 0.0345 | 0.3455 | −0.0825 | 0.0475 | 0.1535 | 0.1575 |
| TGFBR3 | −0.059 | −0.232 | 0.25 | 0.305 | −0.349 | 0.170 |
| THY28 | −0.4985 | −1.0695 | −0.9095 | 0.1665 | −0.4295 | −0.0145 |
| TIF1 | 0.219 | 1.026 | 1.134 | 0.928 | 0.465 | −0.373 |
| TIGA1 | −0.005 | 0.912 | 1.096 | 0.175 | 0.239 | −0.233 |
| TIM14 | 0.1375 | −0.1275 | −0.3325 | −0.0445 | −0.1065 | −0.1895 |
| TIMELES [symbol] | 0.2335 | 0.2335 | 0.1925 | 0.3735 | −0.2665 | −0.1515 |
| TIMM9 | −0.490 | −0.685 | −0.351 | −0.402 | −0.160 | 1.115 |
| TINP1 | −0.1385 | 0.8015 | 1.2275 | −0.1885 | −0.0825 | −0.7445 |
| [symbol] IP1208 | −0.334 | 0.804 | −0.393 | 0.229 | 0.336 | −0.57 |
| TK1 | 1.347 | −0.487 | −0.918 | −0.044 | −0.716 | −1.18 |
| TLE2 | 1.339 | 1.012 | −0.039 | 0.377 | 0.505 | −0.045 |
| TLR5 | 1.235 | 0.705 | 2.132 | 1.421 | 0.081 | 0.598 |
| TM4SF12 | −0.205 | −0.167 | 0.321 | 0.257 | −0.236 | 0.269 |
| TM4SF13 | 0.567 | 0.454 | 0.678 | 0.593 | 0.369 | 0.644 |
| TMEM14A | 0.1285 | 0.0025 | 0.2365 | 0.5555 | 0.0175 | −0.0055 |
| TMEM25 | −0.5755 | 1.1105 | −0.5505 | −0.3385 | 0.3595 | 0.7655 |
| TMEM4 | −0.275 | 0.799 | 0.695 | 0.498 | 0.129 | 0.025 |
| TMPRSS5 | −1.028 | −2.092 | −0.44 | −0.38 | −0.112 | 0.029 |
| TMSB10 | 2.269 | 2.185 | 0.81 | 1.05 | 1.887 | −0.108 |
| TMSNB | 0.1585 | −0.0085 | −0.1755 | 0.0845 | 0.3255 | −0.0455 |
| TNFRSF1 [symbol] | 0.227 | −0.743 | −0.854 | 0.23 | 0.151 | −0.404 |
| TNFRSF2 [symbol] | −0.5415 | 0.8635 | −0.5205 | −1.3715 | 0.1315 | 0.9225 |
| TNFRSF2 [symbol] | 2.0685 | −0.8215 | −0.1385 | 0.1215 | −0.4635 | 1.9495 |
| TNFSF4 | 0.0715 | 0.8875 | −0.2985 | 0.0885 | 0.4265 | 0.1945 |
| TNFSF7 | −0.7785 | 0.1795 | −1.1615 | −0.0695 | 0.0405 | −0.1405 |
| TNIK | −0.7045 | −0.2575 | −0.7355 | 0.3485 | 0.3895 | −0.0995 |
| TNNC2 | −0.441 | 0.502 | −0.288 | −0.936 | −0.021 | 0.334 |
| TNNI2 | 0.222 | −0.001 | 0.058 | −0.663 | 0.232 | −0.647 |
| TNNI3 | −0.8295 | 1.5605 | 1.0415 | 0.5955 | −1.1995 | −0.6535 |
| TOB1 | −0.0925 | −0.6735 | 0.7915 | −0.4545 | −0.0905 | 0.0425 |
| TOM1L1 | −0.7505 | −0.1515 | −1.8935 | −0.2975 | 0.0565 | −0.9985 |
| TOMM7 | −0.3325 | 1.1815 | 0.9345 | 0.9775 | −0.3945 | 0.4415 |
| TOP1MT | −0.359 | 0.442 | 0.493 | −0.298 | 0.204 | 0.254 |
| TOP2A | −0.0225 | 0.2745 | 0.9215 | 0.5855 | 0.3965 | −0.4245 |
| TOP2B | 0.671 | 1.375 | 1.06 | 0.522 | −0.305 | 0.524 |
| TOPBP1 | −1.1095 | 0.8155 | 0.1495 | 0.1765 | 0.0415 | −0.1855 |
| TOPK | 0.4535 | −0.7345 | 0.7565 | −0.4215 | −0.3545 | −0.4195 |
| TP53AP1 | 1.2535 | 0.8135 | −0.5105 | 0.3275 | 0.5545 | 0.3005 |
| TP53BP2 | 1.2475 | −0.0035 | −0.7295 | 0.7645 | 0.0125 | −0.4635 |
| TP53INP1 | 0.9775 | −0.1925 | 0.5015 | 0.4585 | −0.2845 | 0.1455 |
| TPD52L1 | 1.399 | 0.962 | 1.984 | −0.041 | 1.36 | 0.008 |
| TPRT | −0.8795 | 0.2855 | −0.4095 | −0.1305 | −0.9455 | −0.9785 |
| TRA@ | −0.161 | 0.595 | 0.312 | −0.18 | 0.003 | 0.231 |
| TRAF4 | −0.434 | −0.308 | 0.043 | 0.008 | −0.559 | −0.26 |
| TRAPPC4 | −0.46 | −0.445 | −0.63 | −0.163 | −0.358 | −0.199 |
| TRH | −1.2455 | −2.0475 | −1.5265 | 0.4885 | −0.9085 | −0.7785 |
| TRIM17 | 1.0215 | 0.7525 | −0.6465 | 0.3175 | −0.3216 | 0.2075 |
| TRIM2 | −0.8205 | 0.2935 | −0.0725 | 0.2785 | −0.9275 | −0.7835 |
| TRIM6 | −0.051 | −0.685 | −0.276 | −0.585 | −0.549 | −0.173 |
| TRIP3 | 0.4175 | 0.6005 | −0.2335 | 0.3425 | −0.0425 | 0.0025 |
| TRPS1 | 0.3065 | 0.8315 | −0.2545 | −0.3895 | 0.1735 | 0.0185 |
| TSG101 | 0.8045 | 1.0375 | 0.5855 | −0.2105 | −0.0835 | 0.7615 |
| TTC10 | 0.0195 | 0.5875 | 1.3525 | 0.4195 | −0.0915 | −1.0595 |
| TTC12 | 0.159 | 0.132 | −0.186 | 0.035 | 0.438 | 0.147 |
| TTK | −0.4375 | −0.9435 | −1.2555 | 0.1875 | 0.2815 | −0.0035 |
| TTLL4 | −1.316 | −0.574 | −0.845 | −0.491 | 0.011 | 0.372 |
| TTYH1 | −0.4525 | 0.5545 | −0.3965 | −0.5125 | 0.1265 | −0.1785 |
| TUBB3 | 0.0245 | 0.2905 | 0.5325 | 0.3145 | 0.0475 | −0.4535 |
| TULP3 | −0.215 | 1.435 | 1.092 | 0.558 | −0.065 | −0.185 |
| TXN | −0.455 | 0.064 | −0.304 | 0.68 | −0.481 | −0.456 |
| TYMS | 0.3865 | −1.9135 | −0.0945 | −1.8885 | −0.9445 | −0.3145 |
| U5-116KD | 0.8335 | −0.1965 | 0.5765 | 0.4805 | 0.0735 | −0.5145 |
| UBE2E3 | −0.196 | 0.433 | 0.023 | −0.128 | 0.733 | −0.14 |
| UBL5 | −0.0855 | −0.7555 | −0.9195 | −1.2015 | −0.2705 | −0.2155 |
| UBN1 | 0.388 | 0.092 | 0.082 | 0.075 | −0.128 | 0.264 |
| UCHL1 | 1.428 | −0.821 | 0.811 | −0.64 | 0.66 | −0.269 |
| UCHL3 | 0.416 | 0.746 | 1.072 | 0.14 | 0.201 | −0.168 |
| UGCG | 4.1935 | −1.0885 | −0.5195 | 1.1825 | 1.1255 | 2.1555 |
| UGCGL1 | −0.3595 | −0.2825 | 0.1565 | −1.0735 | 0.6945 | −0.6795 |
| UGT2B7 | 0.7775 | 0.0035 | 0.1455 | 0.3735 | 0.5605 | −0.2625 |
| UNC13D | −0.545 | 0.331 | −0.453 | 0.037 | −0.321 | −0.417 |
| VAMP8 | 0.837 | 0.097 | 0.765 | −0.243 | 0.572 | −0.116 |
| VAPA | 0.8945 | 1.6105 | 1.1895 | 0.0155 | 0.6785 | 0.1905 |
| VAV1 | −0.374 | 0.44 | −0.862 | −0.184 | −0.499 | −0.314 |
| VAV3 | −0.524 | 0.193 | 0.648 | −0.512 | −0.196 | 0.135 |
| VCX3 | −0.288 | 0.092 | 0.053 | −0.142 | −0.067 | 0.012 |
| VDAC2 | 0.0485 | −0.4315 | −0.4355 | 0.0125 | −0.0315 | 0.0885 |
| VEST1 | −0.736 | −0.822 | −0.151 | −0.574 | −0.157 | −0.152 |
| VGLL1 | 0.9875 | −0.7785 | −0.5145 | 0.0715 | −0.8115 | −0.5165 |
| VMD2L2 | −0.518 | −0.248 | −0.755 | −0.829 | 0.118 | −0.289 |
| VSX1 | 1.034 | −0.378 | 0.598 | 0.121 | −0.114 | 0.651 |
| WARP | −0.885 | 1.121 | 0.06 | 0.57 | 0.897 | 0.071 |
| WDR26 | 0.7125 | −0.0945 | −0.3805 | −0.1225 | −0.5475 | −0.0185 |
| WDR6 | −0.941 | −0.395 | −0.453 | −0.501 | 0.037 | −0.074 |
| WHSC1L1 | −0.729 | −0.089 | −0.439 | −0.054 | −0.249 | −0.519 |
| WNT3 | −0.4125 | 0.4395 | 1.8575 | −0.3495 | −0.4625 | −0.3395 |
| WNT6 | 0.6595 | 0.0285 | −0.4935 | 0.6105 | 1.0545 | 0.1905 |
| WSB2 | 0.2195 | −0.1025 | −0.0645 | −0.8805 | −0.2535 | −0.3985 |
| WWP1 | 0.6955 | 0.8125 | 0.5585 | 0.8855 | 0.3015 | −0.8165 |
| WWP2 | −0.688 | 0.103 | −0.335 | 0.143 | 0.888 | −0.198 |
| XBP1 | −0.532 | −1.05 | −0.326 | 0.16 | −0.275 | −0.401 |
| XYLT2 | −0.2185 | −0.1605 | −0.5485 | 0.1595 | −0.7165 | 0.0425 |
| YBX2 | 0.742 | 0.196 | 0.96 | 0.526 | −0.101 | 0.336 |
| YIF1 | 0.3245 | 0.8285 | −0.2525 | 0.5235 | 0.2405 | 0.5335 |
| ZA20D3 | 0.6365 | −0.1555 | −0.3375 | 0.0745 | −0.1415 | −0.2585 |
| ZAP128 | 0.0195 | −0.3295 | −0.3725 | 0.3315 | 0.1175 | −0.1385 |
| ZBED3 | −0.379 | −0.728 | 0.442 | −1.138 | −0.764 | −0.614 |
| ZBTB4 | 0.561 | −0.193 | 1.044 | 0.435 | −0.068 | −0.032 |
| ZCWCC1 | 0.4175 | 1.5545 | 0.1745 | 0.7735 | 0.1895 | −0.1685 |
| ZCWCC2 | 0.481 | −0.932 | 0.472 | −0.116 | 0.347 | −1.063 |
| ZDHHC13 | −1.1255 | −0.7235 | −1.1635 | −0.3995 | −0.9145 | 0.2455 |
| ZDHHC14 | −0.5555 | 0.6705 | −0.3185 | 0.6935 | −0.1645 | 0.1105 |
| ZFHX4 | 0.5645 | −0.1325 | −0.0925 | 0.3045 | −0.1735 | 0.1815 |
| ZFYVE21 | −0.42 | −2.96 | −0.033 | 0.329 | −0.135 | −0.848 |
| ZNF133 | −0.136 | 0.267 | −0.618 | 0.63 | 0.337 | −0.105 |
| ZNF165 | 0.3245 | 0.4895 | 0.3245 | 0.0995 | 0.1915 | −0.1965 |
| ZNF24 | 0.391 | −0.122 | 0.189 | 0.572 | 0.177 | 0.15 |
| ZNF318 | −0.0265 | 0.6175 | −0.0125 | −0.0165 | −0.1075 | 0.0355 |
| ZNF54 | 1.1015 | −1.9905 | −2.0045 | −1.7295 | −0.7435 | −0.8925 |
| ZNF432 | 0.7085 | 0.8825 | 0.2835 | 0.3875 | −0.0135 | −0.3055 |
| ZNF482 | 0.73 | 0.199 | 0.463 | 1.869 | 0.338 | 0.644 |
| ZNF505 | 0.455 | 0.209 | −0.026 | 0.55 | −0.209 | −0.117 |
| ZNF552 | 0.1775 | 0.5135 | 0.2115 | 1.3235 | 0.6595 | 0.4975 |
| ZNF577 | 0.2105 | 0.0085 | −0.0205 | 0.1885 | 0.3135 | −0.1595 |
| ZNF587 | −0.7515 | 0.8155 | 0.0085 | −0.0505 | 0.3675 | −0.3495 |
| ZNF598 | 0.4825 | −0.0375 | 0.4765 | 0.4395 | 0.2815 | 0.3095 |
| ZNF599 | 0.203 | 0.688 | −0.231 | 0.283 | −0.314 | 0.853 |
| ZNF606 | −0.564 | −0.356 | −0.054 | −0.004 | −0.594 | −0.068 |
| ZNF614 | 0.2 | −0.033 | −0.527 | 0.139 | −0.09 | −0.231 |
| ZNF639 | | | | | | |
| ZNF7 | | | | | | |

[symbol]

indicates data missing or illegible when filed

TABLE 19

| Symbol | NAME | BR00-028 [symbol] | BR00-036 [symbol] | BR00-057 [symbol] | BR00-058 [symbol] | BR99-020 [symbol] | BR99-034 [symbol] | PB120-LN | PB126 | PB149 | PB205 | PB255 | PB297 | PB311 | PB314 |
|---|---|---|---|---|---|---|---|---|---|---|---|---|---|---|---|
| ABAT | 682378II [symbol] | −0.71 | −0.17 | −1.718 | −0.198 | 2.101 | 1.742 | −2.219 | −2.788 | 2.15 | −1.858 | 1.885 | −1.976 | 0.743 | −1.355 |
| ABC1 | 696882II [symbol] | −1.145 | 0.641 | −1.241 | −0.965 | −0.395 | −0.755 | −1.366 | −0.89 | −0.23 | −1.4 | −0.965 | −0.881 | −0.89 | −1.029 |
| ABCC11 | 697619II [symbol] | 5.574 | 0.322 | −0.343 | 1.524 | 0.38 | 4.056 | −0.302 | −0.699 | 2.28 | −0.405 | 2.032 | 0.031 | 1.395 | 3.009 |
| ABCC3 | 695262II [symbol] | 0.011 | −0.421 | −1.254 | 0.923 | −0.647 | 0.335 | −1.013 | −0.967 | 1.62 | −1.138 | 0.506 | −1.047 | 2.78 | 0.564 |
| ACAS2L | 695930II [symbol] | 0.433 | 2.406 | 1.24 | 1.7 | 1.355 | −0.297 | 0.417 | 0.494 | −0.23 | 0.466 | 0.737 | 0.748 | −1.13 | 1.472 |
| ACAT1 | 686983II [symbol] | −0.442 | 0.088 | 0.524 | 0.334 | 1.118 | 0.508 | −0.758 | −0.683 | 0.09 | −0.917 | −0.157 | −0.093 | −0.895 | −0.32 |
| ACAT2 | 691319II [symbol] | 0.001 | 0.126 | 1.659 | −2.572 | −2.162 | −0.602 | −1.019 | 1.182 | 0.03 | −0.044 | 1.563 | 1.406 | 1.622 | 1.839 |
| ACOX2 | 694368II [symbol] | 2.333 | 0.486 | −1.681 | 2.355 | 0.02 | 0.713 | −1.187 | 0.294 | 1.06 | −0.849 | 0.783 | −0.645 | 1.333 | 1.389 |
| ACOX3 | 690904II [symbol] | 0.419 | 0.278 | −1.859 | −1.15 | −0.07 | −1.288 | −3.312 | −1.98 | 0.3 | 0.828 | −1.685 | −2.11 | 1.411 | −1.525 |
| ACSL3 | 690853II [symbol] | 0.0335 | −0.5205 | −0.2775 | 0.3885 | 0.1215 | −0.5295 | −0.9195 | 0.8395 | 0.55 | −0.9595 | 1.1 | −1.18 | 0.2765 | 0.3205 |
| ACTA1 | 692913II [symbol] | −1.001 | 0.081 | 1.077 | −0.882 | −0.394 | −1.012 | 4.198 | −0.554 | −0.71 | 2.26 | −0.099 | 0.3 | −0.975 | 0.811 |
| ACTB | 695048II [symbol] | 1.344 | 1.374 | −0.125 | 0.665 | 1.528 | 1.371 | 1.832 | 0.514 | −0.33 | 1.703 | 0.792 | 1.032 | 1.315 | 1.037 |
| ADAM33 | 682670II [symbol] | 0.564 | 0.0732 | −0.063 | −0.31 | 0.058 | 0.292 | 0.067 | −0.386 | 0 | −0.083 | 0.181 | 0.236 | −0.11 | −0.271 |
| ADAMDE [symbol] | 687861II [symbol] | −0.553 | 0.231 | −1.838 | 0.372 | 0.169 | −0.819 | 0.041 | 1.537 | −1.19 | −1.483 | −0.075 | 0.352 | −0.642 | −0.398 |
| ADPN | 684643II [symbol] | −0.3975 | −0.4505 | 2.077 | −1.039 | −0.4655 | 2.134 | 1.524 | 1.483 | −0.44 | −0.3715 | −0.5345 | 1.34 | 0.5345 | 0.2945 |
| ADPRHL1 | 681147II [symbol] | −0.587 | 0.833 | −0.382 | 1.076 | 1.855 | −0.224 | −0.058 | −0.098 | 0.11 | 0.169 | −0.59 | −0.633 | 0.786 | −0.42 |
| ADRA2C | 696863II [symbol] | −0.227 | 1.017 | −0.319 | 0.694 | 1.363 | 0.147 | −0.049 | −0.802 | 0.05 | −0.919 | −0.009 | 0.372 | −0.693 | −0.135 |
| AGR2 | 696132II [symbol] | −0.27 | −0.608 | −0.195 | 0.848 | 0.884 | 0.121 | 0.214 | −0.781 | 0.86 | −1.447 | 0.301 | −0.084 | 0.368 | −0.09 |
| AGT | 685839II [symbol] | −0.548 | −1.413 | 1.543 | 1.119 | −0.04 | −0.808 | 0.012 | −1.185 | −0.32 | −0.682 | −0.81 | 1.756 | 0.555 | 1.122 |
| AGTR1 | 683209II [symbol] | −0.3398 | 0.1462 | −0.2918 | 1.483 | 1.397 | 0.0423 | 0.1232 | −0.1038 | 0.79 | −0.1228 | 1.272 | −0.2028 | 0.0183 | −0.3238 |
| AHCYL1 | 681920II [symbol] | −0.792 | −0.384 | −1.227 | 0.535 | 0.656 | 0.234 | 0.026 | −0.813 | 0.34 | −0.424 | 0.26 | 0.294 | −0.251 | −0.287 |
| AHNAK | 695607II [symbol] | 0.219 | 1.117 | −0.329 | 0.091 | 0.707 | 0.908 | −0.093 | −0.719 | 0.45 | −0.48 | 0.269 | −0.879 | −1.545 | 0.627 |
| AHR | 687282II [symbol] | 0.3202 | −0.6219 | −0.8058 | 0.9222 | −0.8408 | 0.2172 | −1.186 | −0.1218 | 0.94 | −1.919 | 0.5442 | −0.9258 | 0.4492 | 0.7602 |
| AIM1 | 692581II [symbol] | 1.166 | 0.2621 | −1.308 | 0.698 | −0.832 | −0.607 | 0.265 | 1.328 | 0.63 | −0.253 | −0.11 | 2.768 | 1.831 | 0.522 |
| AK2 | 686272II [symbol] | 0.585 | −0.518 | −0.096 | 0.181 | 0.01 | −0.557 | 0.397 | 0.381 | −0.07 | 0.498 | −0.01 | −0.507 | 0.548 | 0.219 |
| AK3L1 | 689094II [symbol] | 0.2795 | −0.1075 | 0.0975 | −0.2235 | 0.5605 | −0.1085 | −0.2235 | 0.4725 | 0.21 | −0.8675 | 0.4135 | −1.576 | 0.0555 | 0.0985 |
| AK5 | 686869II [symbol] | −1.138 | −1.394 | −1.842 | 3.196 | 2.427 | −0.717 | 0.821 | −1.423 | 2.97 | −0.785 | 0.809 | −0.803 | −0.787 | 0.046 |
| AKAP1 | 889352II [symbol] | −0.271 | −0.116 | 0.483 | 0.594 | −0.747 | 0.242 | −0.232 | 1.081 | 0.24 | −0.608 | −1.11 | 0.848 | −0.399 | −0.226 |
| AKR1A1 | 681581II [symbol] | 1.717 | 0.517 | −1.272 | 0.002 | 1.317 | 0.141 | −0.002 | −0.51 | −0.18 | −1.002 | 0.233 | −0.445 | 0.017 | 0.08 |
| AKR7A3 | 685301II [symbol] | 0.067 | −0.067 | −0.496 | 1.341 | 0.836 | 0.935 | −0.681 | −0.428 | 0.41 | −0.196 | 0.269 | −0.341 | 1.488 | −0.109 |
| ALG5 | 688178II [symbol] | −0.0385 | −0.6625 | −0.6365 | 0.4045 | 0.1246 | −0.7775 | −0.2975 | −0.3925 | 0.28 | 0.3035 | −0.3385 | −1.148 | 0.1825 | 0.1215 |
| ALG8 | 681787II [symbol] | −0.487 | 0.412 | 0.206 | −0.161 | 0.039 | 0.226 | −0.814 | −0.241 | −0.41 | −0.509 | 0.38 | −0.07 | 3.14 | −0.23 |
| AMFR | 693971II [symbol] | −0.463 | 1.486 | −1.015 | −0.672 | 2.971 | 1.675 | 0.738 | 0.541 | −0.9 | −1.239 | 0.732 | 1.056 | −1.942 | 0.733 |
| AMPD2 | 683330II [symbol] | −0.3317 | −0.2277 | −0.4257 | −0.4967 | 0.5983 | 0.7623 | 0.8743 | −0.2907 | 0.31 | −0.6147 | 0.0053 | 1.28 | 0.0293 | 0.0353 |
| ANAPC11 | 684913II [symbol] | −0.213 | −1.171 | 0.879 | −0.091 | −1.008 | −0.99 | −0.398 | 0.182 | −0.3 | 1.293 | −0.434 | −1.595 | 0.124 | 1.099 |
| ANKRD22 | 691041II [symbol] | 1.222 | 0.307 | 0.883 | −0.19 | −0.681 | −0.727 | −0.426 | 0.114 | −0.57 | −1.541 | 1.065 | −1.059 | 0.4 | 1.557 |
| ANKRD27 | 693340II [symbol] | −0.731 | 1.532 | 1.565 | −0.774 | −0.468 | 0.168 | 0.493 | 0.645 | 0.01 | −0.099 | 0.169 | −0.683 | 0.097 | −0.08 |
| ANLN | 687252II [symbol] | 1.471 | 1.009 | 0.4215 | −0.4215 | −0.4105 | −0.2425 | 2.609 | 2.448 | −0.56 | 2.632 | −0.2065 | 1.58 | 0.9385 | −0.0925 |
| ANP32A | 688123II [symbol] | 0.68 | 2.693 | −1.103 | 3.428 | 3.251 | −0.326 | −0.279 | −0.316 | 0.65 | −0.449 | 2.037 | −0.061 | 1.98 | −0.224 |
| ANXA3 | 885113II [symbol] | 1.979 | −1.097 | −0.652 | −1.193 | −2.541 | 0.042 | 1.981 | 1.342 | 1.13 | 0.976 | 0.384 | −0.21 | 0.002 | −0.647 |
| APBB2 | 685599II [symbol] | −0.458 | 0.21 | −0.38 | 0.444 | 0.772 | −0.039 | −0.948 | −0.778 | 0.17 | −0.687 | −0.379 | −1.06 | 0.342 | −0.927 |
| APOBEC3 | 691683II [symbol] | −0.0346 | 0.4805 | 0.3485 | −0.1125 | −0.6665 | −0.8435 | 4.932 | 3.224 | −0.96 | 0.1885 | 0.9135 | 3.407 | 1.404 | −0.6385 |
| AQP5 | 681185II [symbol] | 0.5905 | −2.625 | 1.991 | −1.283 | −2.33 | −1.33 | 1.983 | 1.903 | 1.23 | −1.351 | −1.83 | 2.108 | −1.588 | −1.583 |
| AR | 693849II [symbol] | 3.194 | 1.476 | −0.592 | 3.056 | 1.491 | 2.54 | −1.514 | −1.879 | 0.32 | −1.737 | 2.161 | −2.498 | 0.998 | 3.448 |
| AREG | 695877II [symbol] | 0.444 | 2.536 | 0.833 | 0.469 | 0.974 | 1.165 | 1.062 | 0.257 | 5.32 | 1.165 | 0.417 | 1.156 | 0.884 | 1.172 |
| ARF1 | 694438II [symbol] | 1.101 | 1.487 | 1.773 | 0.997 | 1.553 | 0.018 | 1.317 | 1.345 | −0.22 | 0.319 | 0.482 | 1.895 | 1.015 | 1.106 |
| ARHGDIB | 694908II [symbol] | 0.734 | −0.961 | −1.839 | 0.646 | 1.115 | 0.038 | 0.384 | −0.201 | 0.24 | 0.029 | 0.174 | 0.996 | −0.208 | 3.825 |
| ARL6IP | 688270II [symbol] | −0.104 | 1.084 | 0.095 | −0.137 | 0 | 0.803 | 0.082 | 0.716 | −0.19 | 0.314 | −0.065 | 0.419 | 0.415 | −0.725 |
| ARL6IP2 | 684285II [symbol] | −0.243 | 0.33 | 1.977 | −0.657 | 0.17 | −0.665 | 0.76 | 2.289 | 0.11 | 0.045 | 0.25 | 0.77 | −0.037 | 0.523 |
| ARMCX2 | 682195II [symbol] | −0.718 | −1.239 | 0.101 | −0.203 | −0.196 | −0.222 | 0.621 | −0.756 | 0.77 | −1.524 | 1.032 | 0.086 | 0.242 | −0.99 |
| ARNT2 | 685876II [symbol] | −0.059 | −0.4406 | 2.325 | 1.98 | 0.604 | −0.37 | −0.445 | −0.584 | 0.5 | −1.808 | 0.523 | −1.444 | 2.587 | 1.489 |
| ARNT2 | 686537II [symbol] | 0.07 | −1.389 | 2.75 | 2.274 | 0.253 | −0.115 | −0.163 | −0.687 | 0.53 | −1.782 | 0.821 | −1.582 | 2.69 | 1.508 |
| ARP3BET [symbol] | 696604II [symbol] | −1.016 | −1.612 | 2.441 | −0.241 | −1.099 | −1.13 | 0.282 | 0.133 | −0.03 | −0.408 | −1.005 | 1.374 | −0.879 | −0.574 |
| ARPC1B | 696647II [symbol] | 0.31 | 0.147 | −0.956 | 0.314 | 0.176 | −0.078 | 1.136 | 0.798 | −0.69 | 0.635 | −0.665 | 0.104 | 0.197 | 0.604 |
| ARPC5L | 690357II [symbol] | −0.0428 | 0.2283 | −1.087 | −0.4377 | 0.5693 | −0.4377 | 1.495 | 0.3823 | −0.07 | 0.2033 | −0.3737 | 0.0493 | −0.5017 | 0.3443 |
| ASB9 | 685235II [symbol] | 0.215 | −2.138 | 2.935 | −0.135 | −0.177 | 0.036 | −0.029 | −0.725 | 0.85 | −0.262 | −0.811 | 0.061 | 0.465 | −0.603 |
| ASF1A | 692334II [symbol] | −0.981 | −0.895 | −0.174 | −1.303 | −0.718 | −1.854 | −0.918 | −0.423 | −0.08 | 0.229 | −1.127 | −0.051 | −0.213 | −1.651 |
| ASH2L | 696184II [symbol] | −0.898 | 1.035 | −0.417 | −0.013 | 0.17 | 0.409 | −0.338 | −0.5 | 0.33 | −0.193 | −0.171 | 0.651 | −0.338 | −0.97 |
| ASK | 686686II [symbol] | −0.205 | −0.834 | 0.5 | −1.271 | −1.419 | 0.394 | 1.394 | 0.751 | −0.35 | −0.047 | 0.673 | 1.352 | 0.316 | −0.231 |
| ASPM | 695669II [symbol] | −1.347 | −1.239 | −0.346 | −2.702 | −2.138 | −2.893 | 0.244 | 0.247 | −1.07 | −1.345 | −2.347 | −0.711 | −1.264 | −1.829 |
| ASS | 695913II [symbol] | 0.085 | 0.42 | −0.189 | 1.334 | 0.54 | −0.788 | 0.602 | 0.313 | −0.12 | −0.307 | −0.464 | 0.125 | −0.576 | 0.136 |
| ATAD2 | 682321II [symbol] | −0.7303 | 1.111 | 0.1197 | −0.2803 | −1.371 | −0.3243 | 1.48 | 3.08 | −1.35 | 1.08 | −0.6883 | 1.262 | 0.2637 | −0.8863 |
| ATF5 | 684294II [symbol] | −0.2135 | 0.0025 | 0.5045 | −0.2325 | 0.4965 | −0.5215 | −0.1185 | 0.2605 | −0.07 | −0.6625 | −0.0345 | −0.8215 | −0.0375 | −0.0545 |
| ATF7IP2 | 681599II [symbol] | −0.6295 | −0.3945 | 0.9635 | −0.9795 | 0.1515 | −0.0805 | 0.3475 | 1.065 | 0.4 | 0.6995 | 0.8915 | −0.2935 | 0.0805 | −0.6805 |
| ATP2B1 | 685531II [symbol] | −0.069 | 1.133 | −1.135 | −0.219 | 0.223 | 0.278 | −0.764 | −0.525 | 0.05 | −0.661 | −0.08 | −0.952 | 0.555 | 0.0603 |
| ATP5G1 | 695474II [symbol] | −1.026 | −0.462 | −0.771 | −0.705 | −0.743 | −1.082 | −1.079 | −0.989 | −0.27 | −0.389 | −1.339 | −0.994 | −1.109 | 0.431 |
| ATP5O | 696801II [symbol] | −0.987 | −0.975 | −0.486 | −0.957 | −0.827 | −1.184 | −0.694 | −0.617 | −0.15 | 0.71 | −0.823 | −0.419 | −0.652 | −1.385 |
| ATP6AP1 | 681830II [symbol] | 0.265 | −0.607 | 1.985 | −0.154 | −0.162 | 1.383 | −0.194 | −0.551 | −0.23 | −0.205 | −0.439 | 0.349 | 0.649 | 0.71 |
| ATP6V0A [symbol] | 681594II [symbol] | 2.193 | 2.706 | 1.538 | −1.707 | −0.716 | 0.043 | −1.784 | 0.864 | −0.32 | −0.258 | 1.134 | 0.894 | −0.087 | −0.883 |
| ATP6V1B2 [symbol] | 692368II [symbol] | 0.769 | 3.467 | 0.516 | 2.762 | 0.969 | 3.025 | 0.237 | 0.709 | 0.44 | 1.795 | 2.515 | 1.383 | 0.527 | 0.433 |
| ATP6V1G [symbol] | 696310II [symbol] | 0.317 | 0.742 | −1.017 | 0.591 | 1.143 | 0.015 | 0.74 | −0.158 | 0.3 | 0.01 | 1.033 | −0.041 | −0.857 | 0.145 |
| ATPIF1 | 692140II [symbol] | 0.798 | −0.107 | −1.511 | 0.126 | 0.562 | −0.007 | 0.202 | −1.348 | 0.28 | −0.849 | −0.054 | −0.16 | 0.189 | −0.59 |
| ATXN10 | 685606II [symbol] | −0.4625 | 0.0485 | −0.0805 | −0.0095 | 0.2835 | 0.1625 | −0.0645 | 1.058 | 0.32 | 0.6575 | 0.2485 | −0.0585 | 0.7625 | 0.0095 |
| B3GNT5 | 683718II [symbol] | −0.571 | −0.868 | 1.947 | −0.27 | 0.16 | −0.463 | 2.328 | 3.117 | −0.72 | 1.584 | −0.325 | 1.499 | −0.247 | −0.62 |
| BACE2 | 697028II [symbol] | 0.674 | −0.982 | 0.9 | −1.325 | −0.432 | −1.198 | −0.299 | 0.577 | 0.01 | −0.613 | 0.558 | 0.462 | 0.082 | 0.095 |
| BAG1 | 689260II [symbol] | 0.412 | −0.091 | −1.759 | 1.025 | 1.923 | 1.163 | −0.782 | −1.732 | 1.13 | −1.524 | 1.601 | −2.124 | −1.508 | −0.041 |

| Symbol | NAME | PB334 | PB362 | PB370 | PB376 | PB413 | PB441 | PB455 | UB29 | UB37 | UB38 | UB39 | UB43 | UB45 |
|---|---|---|---|---|---|---|---|---|---|---|---|---|---|---|
| ABAT | 682378II [symbol] | −1.937 | −1.035 | 0.17 | −2.664 | 0.566 | 3.38 | −1.951 | −1.821 | −2.577 | 1.761 | 1.056 | 1.109 | 2.634 |
| ABC1 | 696882II [symbol] | −1.191 | −1.494 | −0.646 | −0.988 | −1.004 | −0.896 | −0.221 | 0.016 | −1.014 | −1.4965 | −0.084 | −1.621 | −0.944 |
| ABCC11 | 697619II [symbol] | 1.045 | 1.778 | 3.097 | 3.974 | 5.154 | 1.465 | 1.263 | −0.148 | 2.175 | 4.7335 | −0.069 | 1.38 | 4.361 |
| ABCC3 | 695262II [symbol] | −0.614 | 0.338 | −0.167 | −0.228 | −0.81 | −0.775 | 1.879 | 0.324 | 0.019 | −1.356 | −0.343 | −0.766 | 0.634 |
| ACAS2L | 695930II [symbol] | 0.72 | 1.056 | 2.794 | −0.805 | 1.295 | 1.207 | 3.344 | 0.969 | 2.1745 | 0.211 | 1.698 | −0.038 | −0.723 |
| ACAT1 | 686983II [symbol] | −0.98 | −0.575 | 0.506 | −0.007 | 0.669 | −0.185 | 1.092 | −1.464 | −0.4835 | 0.8455 | −1.092 | 0.013 | 0.361 |
| ACAT2 | 691319II [symbol] | 1.374 | 0.881 | 0.548 | 0.379 | −1.345 | −0.214 | −1.05 | 0.709 | −0.0015 | −1.424 | −0.826 | 0.483 | −0.001 |
| ACOX2 | 694368II [symbol] | −2.029 | 0.602 | 1.174 | −0.16 | 0.598 | 2.97 | 0.31 | −1.68 | −0.04 | 1.4795 | 1.158 | −1.451 | 2.66 |
| ACOX3 | 690904II [symbol] | −3.14 | −0.823 | −0.759 | −1.94 | −1.671 | −2.04 | −1.787 | −2.2 | −2.9515 | −1.25 | 0.009 | −0.452 | −0.689 |
| ACSL3 | 690853II [symbol] | 1.042 | −0.0515 | −0.0335 | −0.5825 | 1.159 | −0.5915 | −0.5065 | −1.223 | −0.803 | −0.656 | 0.8245 | −0.0575 | 0.2885 |
| ACTA1 | 692913II [symbol] | −1.083 | −0.065 | −0.746 | −0.783 | −1.265 | 1.085 | −1.871 | −0.301 | 9.861 | −0.531 | −0.54 | 2.181 | 0.443 |
| ACTB | 695048II [symbol] | 0.25 | 0.141 | 1.606 | 1.467 | 0.874 | 0.468 | 1.543 | 1.614 | 1.8985 | 0.424 | −0.063 | 1.404 | 0.593 |
| ADAM33 | 682670II [symbol] | 0.06 | 0.141 | 0.035 | −0.124 | 0.227 | −0.083 | 0.153 | 0 | 0.3235 | 0.8195 | −0.148 | −0.414 | −0.382 |
| ADAMDE [symbol] | 687861II [symbol] | −0.696 | 0.208 | −0.453 | −0.669 | −0.71 | 0.491 | −0.389 | 0.16 | 0.196 | 0.613 | 0.309 | −0.178 | 1.108 |
| ADPN | 684643II [symbol] | −0.3985 | −0.0745 | 1.839 | 2.739 | 1.317 | 1.13 | −1.289 | 0.8575 | 1.334 | −0.3455 | −1.289 | −1.103 | 0.0745 |
| ADPRHL1 | 681147II [symbol] | 0.942 | −0.431 | −0.042 | 0.042 | 1.073 | 0.385 | −0.397 | 0.814 | −0.555 | −0.193 | 0.942 | −0.045 | 1.578 |
| ADRA2C | 696863II [symbol] | −0.229 | −0.035 | 0.407 | −0.778 | 0.432 | −0.062 | −0.085 | −0.306 | −0.1 | 0.697 | 0.654 | 0.375 | 0.268 |
| AGR2 | 696132II [symbol] | −0.887 | 0.197 | 0.313 | −0.571 | −0.193 | 0.084 | −0.613 | −0.449 | −0.3885 | 0.249 | 0.408 | 0.429 | 0.503 |
| AGT | 685839II [symbol] | −0.838 | 0.438 | 0.234 | 0.285 | 0.079 | 2.553 | −0.023 | 1.992 | −0.012 | 2.382 | −1.137 | −1.044 | 0.785 |
| AGTR1 | 683209II [symbol] | −0.5528 | −0.2988 | 0.6602 | −0.1248 | −0.0258 | 1.748 | 0.1312 | −0.1798 | 0.1812 | 0.9682 | 2.742 | −0.0698 | −0.2848 |
| AHCYL1 | 681920II [symbol] | 0.359 | −0.191 | 0.504 | −0.267 | −0.084 | 0.924 | 0.157 | 0.007 | −0.365 | 0.4675 | −0.007 | −0.181 | 0.34 |
| AHNAK | 695607II [symbol] | −0.494 | 1.434 | 1.587 | −0.407 | 0.794 | −0.756 | 0.121 | −0.475 | 1.2205 | −0.003 | 1.014 | 0.952 | −1.48 |
| AHR | 687282II [symbol] | 0.3632 | −0.5178 | 0.0433 | −1.481 | −0.7168 | 0.3002 | −0.3528 | 1.744 | −0.5528 | −0.7328 | −0.7288 | 0.4392 | 0.1542 |
| AIM1 | 692581II [symbol] | 1.085 | 0.663 | 0.919 | 1.308 | −0.406 | 0.583 | 0.649 | 0.927 | 0.543 | −0.805 | −0.259 | 1.546 | 0.287 |
| AK2 | 686272II [symbol] | 0.473 | −0.98 | 0.387 | −0.073 | −0.335 | 0.209 | 0.467 | 0.28 | −0.0325 | −0.6795 | −0.146 | 0.214 | −0.102 |
| AK3L1 | 689094II [symbol] | 0.6145 | 0.5275 | −0.6565 | −0.4705 | 1.332 | −0.4835 | −0.2835 | −0.4735 | −1.278 | −0.302 | 0.2895 | −0.0555 | 0.2935 |
| AK5 | 686869II [symbol] | −1.417 | 1.186 | 0.094 | 0.487 | 2.745 | −0.083 | −1.027 | 1.509 | −0.3775 | 3.904 | −0.982 | 0.785 | −0.618 |
| AKAP1 | 889352II [symbol] | 0.487 | −0.18 | −0.752 | −0.012 | 0.012 | 0.6 | 0.222 | 1.141 | −0.835 | −0.3115 | 0.871 | −0.986 | 0.225 |
| AKR1A1 | 681581II [symbol] | −0.096 | −0.134 | 0.119 | 1.625 | 0.087 | −0.026 | 1.061 | −0.284 | 0.5215 | −0.0975 | −0.284 | 0.663 | −0.276 |
| AKR7A3 | 685301II [symbol] | −0.446 | 0.113 | 1.011 | 0.92 | −0.328 | −0.438 | −0.441 | −1.276 | −0.529 | −0.5155 | 0.437 | −0.303 | 0.883 |
| ALG5 | 688178II [symbol] | 1.319 | −0.5335 | −0.8885 | −0.6435 | 0.6405 | 0.0655 | 0.0245 | −0.4225 | −0.827 | −0.0555 | 0.9595 | 0.5425 | −0.0245 |
| ALG8 | 681787II [symbol] | 0.262 | 2.322 | 0.156 | −0.576 | −0.027 | 0.581 | 0.512 | −0.25 | −0.217 | 0.3505 | 0.027 | −0.28 | 0.488 |
| AMFR | 693971II [symbol] | 0.549 | 0.622 | 0.74 | 0.352 | 3.565 | 0.771 | −0.03 | 0.199 | 0.326 | 0.832 | 1.89 | 2.117 | 1.921 |
| AMPD2 | 683330II [symbol] | 0.2753 | 0.1883 | −0.0098 | −1.065 | 0.5773 | −0.1107 | 0.3843 | 0.2253 | 0.4798 | −0.0053 | −0.2927 | −0.4667 | −0.3127 |
| ANAPC11 | 684913II [symbol] | 0.132 | 0.012 | −0.406 | −0.818 | 0.366 | −0.069 | 0.843 | −1.078 | −0.341 | −0.7175 | −0.012 | 0.478 | 0.065 |
| ANKRD22 | 691041II [symbol] | 1.771 | −1.714 | 0.927 | 0.957 | 0.463 | 0.8 | −0.883 | −0.226 | −0.7355 | −7.35 | −0.347 | 2.667 | 0.01 |
| ANKRD27 | 693340II [symbol] | 0.731 | 0.082 | −0.111 | −0.964 | 0.046 | −0.447 | −0.394 | 0.304 | 1.3365 | −0.475 | 0.138 | −0.135 | −0.33 |
| ANLN | 687252II [symbol] | 1.161 | −1.825 | 0.4695 | 1.365 | −0.7685 | −0.5855 | 0.0925 | 1.056 | 0.264 | −1.006 | −0.9005 | 0.9535 | −0.4355 |
| ANP32A | 688123II [symbol] | −0.502 | 0.861 | 1.975 | −0.165 | 2.057 | 1.783 | 0.309 | −0.624 | −0.683 | 1.333 | 3.379 | 3.772 | 1.013 |
| ANXA3 | 885113II [symbol] | 2.498 | 1.221 | 0.946 | −0.73 | −1.184 | 0.527 | −0.287 | 2.44 | 0.6445 | −0.981 | −0.002 | 1.16 | −0.287 |
| APBB2 | 685599II [symbol] | −1.044 | −0.514 | 0.351 | −0.585 | 0.692 | 1.403 | −0.248 | −0.161 | 0.657 | 0.1135 | 0.998 | 1.235 | −0.415 |
| APOBEC3 | 691683II [symbol] | 3.764 | −1.152 | −1.112 | 2.107 | −0.6735 | −0.6835 | 0.5675 | 1.623 | 1.392 | −0.8585 | −1.367 | 0.0345 | −0.5815 |
| AQP5 | 681185II [symbol] | −0.2685 | 1.273 | −1.423 | 2.675 | −0.9805 | 0.0395 | 3.916 | 0.7485 | 1.043 | 2.342 | −0.7705 | −0.6555 | 0.0595 |
| AR | 693849II [symbol] | 0.938 | 2.533 | 3.681 | 2.948 | 1.352 | 3.757 | 1.407 | −1.683 | 1.2645 | 3.066 | 4.449 | 3.874 | 4.729 |
| AREG | 695877II [symbol] | 0.035 | −0.044 | 1.243 | −0.361 | −0.208 | 0.259 | 2.644 | 0.27 | 1.808 | 0.2515 | 1.12 | 1.449 | 1.348 |
| ARF1 | 694438II [symbol] | 0.922 | 0.274 | 0.357 | 0.546 | 0.713 | 1.074 | 0.861 | 0.641 | 1.0215 | 0.4345 | 0.637 | 1.047 | 0.719 |
| ARHGDIB | 694908II [symbol] | 0.889 | 1.278 | 0.351 | 0.345 | 0.192 | 1.42 | 0.698 | 2.561 | 0.1795 | 0.838 | 0.683 | 1.448 | 0.377 |
| ARL6IP | 688270II [symbol] | 0.188 | −0.17 | −0.155 | −0.223 | −0.509 | 0 | −0.905 | −0.244 | −0.701 | −1.051 | 0.066 | 0.343 | 0.408 |
| ARL6IP2 | 684285II [symbol] | 1.346 | 0.969 | −0.335 | −0.315 | −0.125 | −0.518 | −0.412 | 2.077 | −0.373 | −0.4945 | −0.287 | 0.263 | −0.034 |
| ARMCX2 | 682195II [symbol] | 0.039 | 0.062 | 0.995 | −0.427 | −0.088 | 0.984 | −0.368 | −0.17 | −1.217 | −1.199 | 0.555 | 0.083 | 0.817 |
| ARNT2 | 685876II [symbol] | 0.573 | −1.218 | −1.243 | −2.111 | 0.524 | 1.756 | −1.68 | −1.288 | −1.038 | 1.224 | −0.256 | −0.815 | 0.13 |
| ARNT2 | 686537II [symbol] | 0.84 | −0.956 | −1.083 | −2.091 | 0.908 | 1.859 | −1.813 | −1.278 | −0.5575 | 1.442 | −0.347 | −0.784 | −0.144 |
| ARP3BET [symbol] | 696604II [symbol] | 0.286 | −0.533 | −0.769 | −1.047 | −0.946 | −0.904 | −1.147 | 0.163 | −1.6045 | −1.4035 | −1.087 | −0.702 | −0.285 |
| ARPC1B | 696647II [symbol] | 0.007 | 0.345 | −0.073 | 0.853 | 0.159 | −0.84 | 0.054 | −0.472 | 0.6535 | −1.4485 | −0.86 | 1.014 | −0.508 |
| ARPC5L | 690357II [symbol] | 0.4093 | −0.7697 | −0.2027 | 1.161 | −0.0978 | −0.5927 | −0.4717 | 0.2013 | 1.102 | 0.0428 | 0.4783 | 0.5273 | −0.6097 |
| ASB9 | 685235II [symbol] | 2.304 | 0.392 | −0.188 | 0.747 | −0.184 | 1.315 | 0.094 | 0.527 | −1.008 | −0.2776 | −0.737 | −0.424 | 0.376 |
| ASF1A | 692334II [symbol] | −0.378 | −1.191 | −1.782 | −0.184 | −1.629 | −1.32 | −1.64 | −0.103 | −1.776 | −1.639 | −0.879 | −1.169 | −1.109 |
| ASH2L | 696184II [symbol] | −0.148 | 0.284 | −0.389 | −1.035 | −0.071 | −0.333 | −0.276 | 0.156 | −0.665 | −0.654 | −0.261 | 0.347 | −0.01 |
| ASK | 686686II [symbol] | 0.747 | 0.731 | 0.104 | −0.885 | −0.571 | −0.742 | 0.264 | −0.599 | −0.6275 | −1.161 | −0.54 | 1.043 | 0.719 |
| ASPM | 695669II [symbol] | −0.4 | −2.412 | −2.161 | −1.465 | −2.838 | −2.435 | −2.177 | −1.362 | −1.4305 | −2.5495 | −3.28 | −2.034 | −2.96 |
| ASS | 695913II [symbol] | 1.036 | −0.085 | −0.166 | −0.297 | −0.221 | 0.071 | −0.267 | −1.047 | 0.2705 | 0.639 | −0.403 | −0.368 | −0.92 |
| ATAD2 | 682321II [symbol] | 0.6897 | −1.03 | 0.3657 | 1.291 | 0.2597 | −0.1853 | 0.9397 | 0.8997 | −0.5113 | −0.8518 | −0.3703 | 0.1797 | −0.0103 |
| ATF5 | 684294II [symbol] | 0.0275 | 0.3675 | 0.5525 | −0.0205 | −0.5185 | −0.3155 | 0.3575 | 1.114 | 1.65 | −0.0005 | 0.0205 | 0.1755 | 0.1355 |
| ATF7IP2 | 681599II [symbol] | 0.4335 | 0.1435 | −0.2355 | −0.4485 | 1.943 | −0.8495 | 0.4745 | 0.7085 | −0.2845 | −0.113 | 0.3655 | −0.4825 | 0.5115 |
| ATP2B1 | 685531II [symbol] | 0 | 0.437 | 0.101 | −0.314 | −0.259 | −0.381 | 0.001 | −0.131 | −0.411 | −1.001 | 0.375 | 0.184 | 0.1 |
| ATP5G1 | 695474II [symbol] | −0.285 | −1.226 | −1.322 | −1.608 | −0.906 | −0.951 | −1.017 | −1.846 | −0.9945 | −1.5755 | −0.95 | −1.241 | −0.75 |
| ATP5O | 696801II [symbol] | −0.183 | −1.178 | −0.926 | −0.387 | −0.935 | −0.802 | −1.235 | −1.049 | −1.1955 | −0.806 | −1.057 | −0.613 | −0.605 |
| ATP6AP1 | 681830II [symbol] | −0.068 | 0.152 | −0.567 | 1.01 | −0.064 | −0.22 | 0.817 | 1.346 | 0.2365 | −0.4715 | 0.07 | −0.807 | −0.714 |
| ATP6V0A [symbol] | 681594II [symbol] | −0.1 | −0.043 | 0.25 | 0.448 | −0.619 | −1.157 | −0.996 | 0.628 | −0.958 | −0.198 | 0.16 | 0.309 | −1.894 |
| ATP6V1B2 [symbol] | 692368II [symbol] | 1.298 | 0.523 | 1.668 | 0.621 | 0.922 | 0.912 | 0.349 | 1.745 | 0.344 | 1.428 | 1.674 | 2.614 | 3.447 |
| ATP6V1G [symbol] | 696310II [symbol] | 0.054 | −1.259 | 0.514 | 1.241 | 0.788 | 0.094 | −0.275 | −0.21 | 0.9105 | 0.2665 | 2.608 | 1.174 | 0.034 |
| ATPIF1 | 692140II [symbol] | −0.455 | −1.964 | 0.013 | 0.092 | 0.144 | 0.333 | −1.322 | −1.227 | −0.257 | 0.555 | 0.141 | −1.218 | −0.866 |
| ATXN10 | 685606II [symbol] | 0.3135 | −0.1405 | −0.1715 | −0.1555 | 0.2025 | −0.1525 | −0.3595 | −0.5615 | −0.25 | −0.453 | −0.3925 | 0.7715 | 0.6985 |
| B3GNT5 | 683718II [symbol] | 2.526 | 1.026 | −0.315 | 0.776 | −0.469 | −0.141 | −0.459 | 3.211 | 0.252 | 0.283 | −0.523 | 0.151 | −0.261 |
| BACE2 | 697028II [symbol] | 0.465 | 0.262 | −0.646 | 0.992 | −0.262 | −1.285 | 0.79 | 1.137 | 0.11 | −0.22 | −0.982 | −0.503 | −0.792 |
| BAG1 | 689260II [symbol] | −1.085 | 0.041 | 0.174 | −1.161 | 2.585 | 0.216 | −0.492 | −1.653 | −0.3835 | 1.26 | 0.477 | 0.782 | −0.51 |

| Symbol | NAME | BR00-028 [symbol] | BR00-036 [symbol] | BR00-057 [symbol] | BR00-058 [symbol] | BR99-020 [symbol] | BR99-034 [symbol] | PB120-LN | PB126 | PB149 | PB205 | PB255 | PB297 | PB311 | PB314 | PB334 |
|---|---|---|---|---|---|---|---|---|---|---|---|---|---|---|---|---|
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| BIRC2 | 685336II [symbol] | −0.1325 | −0.3775 | 0.7935 | −0.1425 | −0.4995 | −0.2885 | 3.997 | 1.114 | 0.45 | −1.027 | −0.1125 | −0.0265 | −0.4865 | 0.0105 | 0.5285 |
| BIRC5 | 687015II [symbol] | 1.049 | 0.2395 | 0.5215 | 0.0475 | −0.7005 | −0.2405 | 2.289 | 2.47 | −1.03 | 1.95 | −0.5705 | 1.994 | 1.318 | −0.9995 | 0.9535 |
| Bit1 | 685275II [symbol] | 0.5935 | 0.2875 | −0.0125 | −0.0625 | 0.2965 | −0.2595 | −0.0625 | 0.6995 | 0.03 | −0.1015 | −0.4815 | 0.0005 | 1.004 | −0.6415 | 0.5505 |
| BIVM | 691115II [symbol] | −1.079 | 0.0215 | 1.202 | −0.3785 | −0.7585 | −0.9115 | 0.0895 | −0.2605 | 0.02 | −0.4625 | 0.2235 | 0.0995 | −0.0895 | 0.3775 | 1.291 |
| BLVRA | 695027II [symbol] | 0.034 | 0.369 | −2.259 | 0.264 | 0.62 | −0.332 | −1.041 | −1.605 | 0.08 | −1.248 | −0.22 | −2.495 | 0.299 | 0.038 | −0.825 |
| BM039 | 692354II [symbol] | −2.4 | −1.8 | −3.773 | −2.78 | −2.738 | −3.25 | −1.516 | −1.04 | −0.32 | −0.272 | −2.979 | −2.546 | −1.773 | −3.388 | −1.428 |
| BMP7 | 697100II [symbol] | 1.072 | 1.064 | 0.911 | −0.01 | −0.398 | 0.07 | 1.072 | 1.276 | −0.15 | 0.618 | 1.024 | 1.561 | 1.774 | 1.108 | 0.764 |
| BOC | 694925II [symbol] | −0.12 | 0.667 | 0.618 | −0.532 | −0.218 | 0.089 | 0.571 | 0.316 | 0.57 | 0.339 | 0.017 | 0.469 | 0.372 | −0.169 | 1.262 |
| BRCA1 | 691229II [symbol] | 0 | −0.371 | 0.12 | 0.51 | −0.653 | −0.08 | 0.83 | 0.12 | −0.68 | 1.82 | −0.458 | 0.752 | 0.484 | −0.449 | −1.15 |
| BRF2 | 697749II [symbol] | −0.677 | 1.016 | 0.61 | 0.255 | 0.202 | 0.806 | 0.118 | 0.018 | −0.1 | 0.682 | 0.52 | 1.621 | 0.111 | 0.472 | 0.537 |
| BRI3 | 694633II [symbol] | 0.357 | 0.349 | 1.113 | 0.227 | −0.352 | 0.339 | 1.206 | 0.95 | −0.21 | 1.135 | 0.606 | 0.327 | 0.143 | −0.203 | 0.266 |
| BRI3BP | 685224II [symbol] | −0.0885 | 0.0855 | 0.5615 | −0.1285 | 0.3545 | −0.8245 | −0.0925 | 0.1595 | −0.68 | −0.0975 | 0.0815 | −0.4875 | −0.0795 | 0.1645 | 1.228 |
| BSPRY | 689357II [symbol] | 0.5895 | −0.6785 | −0.8455 | 1.026 | −0.6525 | 1.408 | 0.9685 | −0.0245 | 0.32 | −2.148 | 0.3205 | −1.158 | 0.2345 | 0.9365 | 0.6955 |
| BTF3 | 686148II [symbol] | −0.378 | −0.157 | 0.184 | 0.642 | 0.195 | −0.191 | −0.217 | −0.306 | 0.42 | −0.108 | 0.233 | −1.506 | 0.004 | −0.038 | −0.607 |
| BTG3 | 681935II [symbol] | 0.49 | −0.719 | 0.215 | −1.504 | −0.259 | 0.044 | 0.701 | 2.078 | −0.28 | 0.871 | −0.046 | 1.627 | −0.566 | 0.052 | 1.76 |
| BTN2A2 | 685872II [symbol] | 0.0828 | −1.662 | 0.0658 | −0.4062 | 1.354 | 0.3608 | 0.4538 | −0.0303 | −0.45 | −2.194 | −0.2422 | 0.0288 | 0.1438 | −0.7142 | 1.069 |
| BUB1 | 682711II [symbol] | 0.5365 | 0.7295 | 1.523 | −0.6625 | −0.2985 | −0.5025 | 2.029 | 2.428 | −1.24 | 1.689 | 0.0075 | 1.703 | 0.5335 | 0.1865 | 0.6655 |
| BUB3 | 683752II [symbol] | −0.0085 | 0.0085 | −0.3815 | 0.0125 | −0.2145 | 0.5695 | −0.1725 | −0.0215 | −0.24 | 0.7755 | 0.7275 | −0.2525 | 0.2795 | 0.8115 | 0.1635 |
| BXDC1 | 686690II [symbol] | 0.648 | −0.766 | 1.012 | −0.281 | −0.267 | −0.182 | 0.144 | 0.403 | −0.06 | 0.663 | −0.79 | 0.697 | 1.112 | −0.0741 | 0.729 |
| BZRP | 682336II [symbol] | 0.7895 | 0.1835 | 0.3685 | 0.4415 | −0.3725 | 0.0385 | 0.3555 | 1.075 | −0.4 | −0.7285 | −0.4705 | 0.9095 | 0.4215 | 0.6325 | 0.5325 |
| BZW1 | 689148II [symbol] | 0.19 | 0.2 | −0.511 | 0.39 | 0.318 | 0.327 | −0.623 | −0.193 | 0.11 | −0.13 | 0.613 | −0.662 | 0.417 | 0.438 | 0.09 |
| C10orf116 | 694348II [symbol] | 3.641 | 3.615 | 2.311 | 3.161 | 0.644 | 3.618 | −0.517 | 0.024 | 0.12 | −0.072 | 3.711 | 0.389 | 2.969 | 3.03 | 0.545 |
| C10orf38 | 696429II [symbol] | −0.315 | −0.262 | 2.471 | −0.686 | 0.37 | −0.898 | 0.554 | 1.218 | 0.33 | −0.55 | −0.569 | 0.89 | −0.568 | −0.692 | 0.951 |
| C10orf4 | 682455II [symbol] | −0.189 | −0.385 | 1.011 | −0.757 | −0.559 | −1.052 | −0.141 | 0.632 | 0.24 | −0.18 | −1.043 | −0.606 | −0.581 | −0.125 | 0.95 |
| C18orf45 | 696197II [symbol] | 8.513 | 0.78 | −0.131 | 0.436 | 2.162 | 1.786 | 0.413 | 1.436 | −0.94 | −0.332 | 1.054 | 0.013 | −0.686 | 0.276 | −0.131 |
| C10orf7 | 688194II [symbol] | −0.31 | −0.179 | 0.87 | −0.155 | 0.48 | −0.019 | 0.364 | 0.906 | −0.07 | 0.282 | −0.16 | 0.809 | 0.285 | −0.007 | 1.393 |
| C10orf82 | 693231II [symbol] | −0.183 | 1.377 | −0.671 | 1.237 | −0.153 | 0.76 | −0.251 | −0.006 | 0.19 | 0.528 | 2.231 | 0.074 | 1.966 | −0.051 | −0.429 |
| C11orf24 | 694704II [symbol] | 0.16 | −0.49 | −0.2 | −0.28 | 0.128 | −0.295 | −0.364 | −0.672 | −0.38 | −0.085 | −0.214 | 0.471 | −1.067 | 0.142 | −0.09 |
| C12orf14 | 692853II [symbol] | −0.546 | −1.421 | 0.264 | −1.325 | −0.906 | −1.037 | −0.712 | −1.042 | −0.21 | 0.594 | −1.952 | −0.489 | −0.356 | −0.86 | −0.662 |
| C13orf21 | 697705II [symbol] | 0.956 | 0.214 | 0.209 | 1.432 | 3.293 | 1.782 | 1.071 | 0.277 | 0.95 | 0.133 | 1.051 | 0.37 | −0.485 | 0.1 | 0.46 |
| C13orf23 | 691314II [symbol] | 0.1855 | 1.058 | 0.1605 | −0.7525 | 0.0435 | −1.117 | 0.3725 | 0.8005 | −0.37 | 0.9235 | −0.7175 | 0.0315 | −0.4435 | −0.0315 | 1.328 |
| C14orf94 | 694118II [symbol] | −0.5595 | 0.8975 | −1.678 | −0.5415 | 0.7595 | −0.5195 | −0.0085 | −0.8595 | 0.05 | −1.496 | 0.9255 | 0.0085 | 0.2955 | −0.0805 | −0.0325 |
| C16orf23 | 681265II [symbol] | 0.0165 | 0.8235 | 0.7275 | −0.0155 | 0.2535 | −0.2545 | 1.416 | 1.744 | −0.46 | 2.09 | 0.2095 | 0.3935 | 0.6315 | 0.1255 | 0.9425 |
| C16orf45 | 772706II [symbol] | 2.783 | 0.0285 | −0.5365 | −0.3625 | −0.3415 | −0.0905 | 0.2325 | −0.5415 | 1.24 | −0.3725 | −0.0515 | 0.5965 | 0.1215 | −0.1545 | 2.43 |
| C17orf37 | 686313II [symbol] | 1.724 | −0.5812 | −0.9442 | 0.4508 | 0.0358 | 0.2288 | −0.7702 | 0.3338 | −0.4 | −1.065 | 0.1088 | 0.2088 | −0.1962 | 2.258 | −0.5932 |
| C18orf1 | 684420II [symbol] | 0.083 | 0.877 | −0.364 | 0.006 | −0.532 | 0.364 | −0.199 | −0.022 | 0.65 | −1.665 | −0.28 | 0.502 | −0.832 | 0.135 | −0.291 |
| C19orf10 | 687290II [symbol] | −0.4105 | −0.8875 | 2.95 | −0.4975 | −1.285 | 1.329 | 0.0405 | 0.2875 | −0.34 | −0.3265 | 0.7245 | 1.62 | 0.3525 | −0.0955 | −1.152 |
| C19orf32 | 697502II [symbol] | 1.352 | 1.069 | −0.875 | 0.992 | 0.428 | 1.274 | −0.357 | −0.856 | 0.79 | 0.146 | 1.105 | −0.068 | 1.164 | 1.733 | −0.791 |
| C19orf33 | 693018II [symbol] | −1.003 | 1.255 | −1.358 | 1.156 | −0.93 | 1.433 | −0.799 | −0.701 | 0.53 | −0.977 | 0.968 | −1.295 | 0.603 | −0.893 | −0.727 |
| C1orf24 | 689493II [symbol] | −0.2845 | 0.0905 | 0.6395 | 1.249 | 0.5195 | 0.0225 | −0.7825 | −1.205 | −0.26 | −4.476 | −0.0355 | −0.5185 | 0.6055 | −0.1175 | −1.339 |
| C1orf34 | 694666II [symbol] | 0.581 | 2.5 | −1.459 | −0.603 | 0.347 | 2.795 | 0.033 | 0.208 | 0.79 | −1.128 | 3.385 | −0.353 | 1.53 | 0.018 | −1.624 |
| C20orf103 | 695553II [symbol] | 1.368 | 3.33 | 0.668 | 1.795 | 2.213 | 0.552 | 0.826 | −0.701 | 1.69 | 0.767 | 3.719 | −0.022 | 1.217 | 1.844 | 0.241 |
| C20orf149 | 691805II [symbol] | 0.2445 | 0.5675 | −0.2525 | 0.0615 | −0.0205 | 0.0985 | −0.6205 | −0.4845 | −0.36 | −0.4385 | 0.8825 | 0.0825 | −0.5095 | −0.3815 | −1.351 |
| C20orf23 | 697940II [symbol] | −0.231 | 2.075 | 0.221 | 2.718 | 1.144 | 1.333 | 0.977 | 1.33 | 0.31 | −1.136 | 2.008 | 0.136 | 1.33 | 1.769 | 0.225 |
| C20orf3 | 685459II [symbol] | −0.606 | 0.263 | −0.02 | 0.359 | 0.596 | 0.757 | 0.194 | 0.528 | 0.33 | −0.369 | 0.326 | 0.155 | −0.585 | 0.258 | −0.627 |
| C20orf35 | 693279II [symbol] | −0.667 | 1.143 | 0.582 | −0.147 | −0.255 | −0.001 | 0.566 | 1.421 | 0.27 | 1.128 | 0.462 | 0.184 | 0.28 | −0.42 | 0.474 |
| C20orf43 | 687406II [symbol] | −0.1345 | 0.3645 | 0.0665 | 0.1045 | −0.4175 | 0.2925 | −0.2975 | −0.2225 | −0.05 | 1.389 | 1.215 | −0.0185 | 0.2005 | −0.2535 | −0.0145 |
| C20orf6 | 688651II [symbol] | −0.435 | −0.116 | 1.032 | −0.317 | −0.945 | −0.386 | −0.657 | 0.678 | 0.02 | 0.461 | −1.556 | 0.046 | −0.118 | −0.331 | 0.31 |
| C21orf45 | 681352II [symbol] | 1.589 | −0.165 | −1.71 | −1.305 | 0.619 | −1.278 | −0.024 | −1.908 | −0.78 | −1.108 | −0.96 | −0.807 | −0.524 | −0.955 | 0.908 |
| C22orf18 | 681943II [symbol] | 0.445 | 0.532 | −0.645 | −0.187 | 0.964 | −0.306 | 0.358 | 1.704 | −1.04 | 1.205 | −0.022 | 1.251 | 0.112 | −0.035 | 0.131 |
| C2F | 687401II [symbol] | −0.25 | 0.404 | 2.372 | 0.154 | −0.205 | −0.015 | −0.191 | 0.112 | −0.12 | 1.813 | −0.051 | 0.55 | 0.297 | −0.336 | −1.222 |
| C2orf17 | 693373II [symbol] | 0.532 | −0.369 | −0.271 | 0.705 | 0.614 | 1.119 | 0.175 | −0.24 | 0.48 | −0.298 | 1.007 | 1.169 | −0.012 | 0.412 | 0.605 |
| C4A | 689714II [symbol] | −0.639 | −1.303 | −1.668 | 1.694 | 0.517 | 0.237 | −1.518 | −1.33 | 0.72 | −1.461 | 0.139 | −0.967 | 0.348 | 0.712 | −0.618 |
| C5orf13 | 694921II [symbol] | 1.071 | −0.542 | 0.191 | −1.044 | −1.427 | −1.778 | −0.455 | −1.066 | 0.04 | −0.853 | −0.158 | −1.625 | −0.263 | −0.112 | −1.662 |
| C6orf108 | 694989II [symbol] | −0.788 | −0.158 | 0.787 | −0.478 | 0.778 | −0.401 | 0.098 | −0.694 | −0.39 | −0.8 | −0.382 | −0.045 | 0.416 | −0.901 | −0.148 |
| C8orf166 | 694215II [symbol] | 0.45 | 0.05 | 0.655 | 0.237 | 0.034 | −0.143 | 0.468 | 0.041 | −0.16 | −0.243 | 0.047 | 1.033 | 0.18 | −0.403 | 0.705 |
| C8orf182 | 691969II [symbol] | 0.066 | −2.172 | 0.808 | −0.191 | −0.123 | −0.217 | −0.034 | −0.059 | 0.47 | −0.118 | −0.396 | −0.361 | 0.467 | 0.841 | 1.306 |
| C6orf211 | 692990II [symbol] | 0.584 | −1.255 | 1.204 | −0.366 | −0.895 | −0.702 | 1.514 | 1.944 | 1.5 | 1.634 | −0.683 | 0.028 | 0.889 | 0.136 | 1.204 |
| C6orf49 | 692808II [symbol] | −0.55 | −0.695 | −0.444 | −0.351 | −0.554 | −0.576 | −0.686 | −0.598 | −0.17 | 0.169 | −0.869 | 0.111 | −0.374 | −0.51 | −0.133 |
| C6orf85 | 693055II [symbol] | 1.341 | −0.133 | −0.465 | −1.064 | −0.804 | 1.553 | −1.267 | −2.196 | 0.79 | −1.291 | −0.565 | −1.551 | −1.144 | 1.33 | −0.512 |
| C7orf24 | 685020II [symbol] | 0.9915 | 1.004 | −0.2495 | −0.5335 | 0.7125 | −1.017 | −0.0645 | 0.2655 | −0.25 | 0.2795 | 0.3575 | −0.3925 | 0.0195 | 0.9945 | 0.5775 |
| C9orf10 | 682761II [symbol] | 1.724 | 0.842 | 0.77 | 1.586 | 0.79 | 1.58 | 1.39 | 1.146 | −0.32 | −0.416 | 1.154 | −0.235 | 1.679 | 0.762 | 0.713 |
| C9orf121 | 692585II [symbol] | −2.095 | −0.285 | −2.23 | −0.443 | −1.051 | −0.525 | −1.422 | −1.594 | 1.62 | −1.586 | 1.291 | −1.934 | −0.37 | −2.266 | −2.122 |
| C9orf58 | 686494II [symbol] | 1.15 | −0.6305 | 1.236 | −2.076 | −0.8995 | −0.8965 | 3.25 | 0.8765 | −0.22 | 2.528 | 0.0995 | 2.95 | −0.4875 | −0.6945 | 1.421 |
| C9orf85 | 685307II [symbol] | −0.068 | 0.345 | 0.101 | −0.195 | 0.556 | −0.119 | −0.302 | 0.131 | −0.21 | −0.324 | −0.292 | −0.545 | −0.124 | −0.196 | −0.09 |
| C9orf9 | 696998II [symbol] | 0.6155 | −0.0075 | 0.3825 | −0.6865 | −0.2745 | −0.3575 | −0.1245 | 0.0435 | −0.05 | 0.4575 | −0.8925 | 0.7555 | −0.2585 | 0.0845 | −0.0805 |
| CA12 | 697341II [symbol] | 1.233 | 1.186 | −1.496 | 2.581 | 1.084 | 3.039 | −1.422 | −0.795 | 0.59 | 1.835 | 2.611 | −1.369 | 1.146 | 0.285 | −1.15 |
| CA8 | 681755II [symbol] | 1.008 | −0.0195 | −0.4715 | 1.714 | 0.1865 | −0.1105 | 0.0195 | 2.09 | 0.79 | −0.8595 | 0.3535 | 2.126 | −0.7825 | 1.612 | 1.37 |
| CA8 | 683844II [symbol] | 0.8145 | 0.3925 | −0.5415 | 1.346 | 0.4145 | −0.4555 | −0.2155 | 2.384 | 0.64 | −1.661 | 0.0445 | 2.199 | −0.8515 | 1.217 | 1.515 |
| CACNA1 [symbol] | 687482II [symbol] | 0.421 | 0.134 | 0 | −0.044 | 0.309 | −0.023 | −0.598 | −0.012 | −0.02 | 0.188 | −0.066 | −0.067 | −0.205 | −0.366 | −0.158 |
| CALCA | 686738II [symbol] | −0.12 | −0.009 | 1.377 | −0.071 | −0.085 | 0.348 | −0.013 | 0.102 | −0.18 | 0.116 | 0.185 | 0.342 | −0.062 | −0.087 | 0.12 |
| CALML4 | 685502II [symbol] | −0.3535 | −0.1675 | 0.4635 | −1.095 | 1.48 | −0.4515 | 0.0685 | 0.4025 | −0.01 | −0.0625 | −0.4715 | 0.8415 | −0.6005 | 0.2836 | 0.6025 |
| CALML5 | 686121II [symbol] | 4.238 | 0.85 | 0 | 0.803 | −0.623 | 0.063 | 1.307 | 1.751 | −0.34 | −0.646 | −0.396 | −0.476 | −0.745 | 0.551 | 2.728 |
| CaMKIIN [symbol] | 691045II [symbol] | −0.4 | 0.732 | −1.616 | 0.376 | −1.223 | 0.593 | −0.843 | −2.093 | 1.41 | −1.361 | 0.84 | −0.816 | 1.176 | 1.222 | −1.812 |
| CAP2 | 693946II [symbol] | −0.779 | 0.774 | 1.165 | −0.303 | −1.003 | 1.461 | −0.158 | −0.178 | 1.05 | −0.469 | 1.766 | 0.434 | 0.834 | −0.539 | −0.745 |
| CAP350 | 695521II [symbol] | −0.405 | 0.146 | 0.747 | 0.022 | −0.197 | −0.946 | 0.124 | 0.401 | −0.05 | −1.091 | −0.467 | −0.071 | −0.106 | −0.172 | 0.371 |
| CAPN13 | 691887II [symbol] | 1.446 | −0.5283 | −1.274 | 2.456 | −0.3153 | 1.169 | −0.3903 | −1.299 | 1.06 | −1.539 | 0.7217 | −1.022 | 1.005 | 0.2817 | −1.354 |
| CAPN6 | 697168II [symbol] | 0.018 | 0.693 | −1.75 | −0.256 | −2.012 | 0.013 | −0.894 | 0.296 | 1.6 | −2.586 | 0.099 | 0.47 | −1.499 | −1.204 | 0.965 |
| CARD14 | 681056II [symbol] | 0.445 | 0.484 | −0.727 | −0.757 | −0.586 | 0.171 | −0.185 | 1.056 | −0.09 | −1.458 | −0.282 | 0.09 | 0.979 | 0.352 | −0.683 |
| CART | 687059II [symbol] | −0.0455 | 1.097 | −0.2045 | 5.46 | 0.4365 | −0.1435 | −0.0325 | 0.1045 | 0.71 | −0.1045 | −0.0515 | −0.0085 | 0.0025 | −0.1125 | −0.0525 |
| CASP1 | 682686II [symbol] | −0.185 | −1.241 | −0.435 | −1.072 | 0.951 | −1.696 | −0.026 | 0.239 | 0.61 | −1.395 | 0.64 | −0.099 | 0.281 | 0.278 | 1.38 |
| CASP3 | 696606II [symbol] | 0.079 | 0.196 | −0.434 | 0.066 | −0.314 | 0.105 | 0.449 | 0.466 | −0.13 | 0.2 | −0.045 | 0.062 | 0.017 | −0.139 | 0.033 |
| CASP7 | 693491II [symbol] | 1.345 | 0.978 | −0.582 | −0.061 | −0.169 | 0.866 | 0.171 | 0.911 | −0.33 | −0.181 | 1.277 | −0.337 | −0.121 | 0.907 | 0.771 |
| CBLN1 | 685274II [symbol] | −0.2213 | −0.2363 | 1.1 | 0.2297 | 0.4197 | −0.1053 | −0.4693 | −0.2823 | 0.12 | 0.4137 | 0.8647 | −0.1733 | −0.1603 | 0.0338 | −0.3893 |
| CBR1 | 764397II [symbol] | 1.379 | 2.154 | 0.046 | 1.381 | 1.841 | 0.657 | 1.244 | 1.163 | 0.18 | −0.366 | 0.37 | 1.965 | 0.341 | 1.472 | −0.055 |
| CBR3 | 683488II [symbol] | 1.097 | −1.725 | −0.7225 | 0.4765 | 1.09 | −1.189 | 0.2615 | 0.3825 | 0 | −2.515 | −0.2305 | 0.6645 | −1.224 | 0.1475 | 0.9095 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| CBR4 | 697391II [symbol] | −0.575 | −1.474 | −1.577 | −0.795 | −0.843 | −0.181 | −1.03 | −0.986 | 0.37 | −0.811 | −0.833 | −0.941 | 1.057 | 0.426 | −0.384 |
| CCL24 | 681864II [symbol] | −0.013 | 0.09 | 0.238 | −0.005 | 0.463 | 0.047 | −0.139 | −0.106 | 0.15 | −0.323 | 0.092 | −0.116 | −0.3 | −0.02 | −0.139 |
| CCL28 | 685513II [symbol] | 2.981 | −0.453 | −2.854 | −0.027 | −0.131 | −0.132 | −0.943 | 0.655 | 0.34 | −2.235 | −1.018 | 1.275 | −1.331 | 0.11 | 2.488 |
| CCNB2 | 694924II [symbol] | −1.171 | 0.553 | −1.049 | −0.462 | −0.998 | −0.233 | −0.404 | −0.057 | −0.78 | 0.097 | −0.249 | −0.358 | −0.956 | −0.391 | −0.554 |
| CCND1 | 689117II [symbol] | 0 | 0.329 | 0.684 | 0.972 | 1.019 | 0.388 | −2.428 | −2.605 | −0.49 | −1.252 | −0.325 | −2.387 | 2.609 | −0.069 | 0.516 |
| CCNE1 | 685647II [symbol] | 0.2988 | −0.1122 | 1.805 | −0.7812 | 0.2888 | 0.1118 | 3.452 | 1.272 | −0.74 | −4.09 | −0.2792 | 3.026 | 0.7768 | 0.2098 | 0.3508 |
| CCNG2 | 686672II [symbol] | −0.9365 | 0.5265 | −0.4275 | 0.7445 | −0.5725 | 0.9125 | −1.086 | 0.2105 | 0.89 | −0.6555 | 1.361 | −0.6435 | −0.5545 | 0.6375 | −0.7325 |
| CCNH | 688140II [symbol] | −0.6995 | −1.165 | 1.927 | 0.3545 | 0.2155 | 0.6185 | 1.991 | −0.1385 | −0.01 | −0.4695 | 0.1205 | 0.6715 | −0.3965 | −0.3175 | 1.382 |
| CD38 | 691273II [symbol] | 1.626 | −0.539 | −1.1 | −0.567 | 0.911 | 0.105 | 0.517 | −0.066 | −0.61 | −0.458 | 0.158 | 0.425 | 0.638 | 0.208 | 2.428 |
| CD3D | 696702II [symbol] | −1.517 | −3.339 | −3.816 | −3.427 | −2.101 | 3.083 | −1.61 | −3.517 | −0.1 | −3.526 | −2.222 | −2.781 | −2.311 | −2.866 | −1.537 |
| CD4 | 691128II [symbol] | 1.359 | −0.138 | −0.214 | −0.35 | 0.405 | −0.254 | 0.002 | −0.344 | −0.39 | −0.33 | 0.119 | 0.434 | 0.05 | −0.045 | 1.114 |
| CD58 | 695421II [symbol] | −0.488 | −1.939 | −1.424 | −0.734 | −1.204 | −1.166 | −0.3998 | −0.686 | 0 | −1.502 | −1.124 | −0.711 | −0.321 | −0.658 | 0.375 |
| CD59 | 682450II [symbol] | −0.2545 | −0.3655 | −0.6185 | 0.8465 | 0.3225 | 0.3685 | −1.018 | 1.546 | 0.57 | −1.062 | 0.2585 | −0.0285 | −1.244 | −0.5615 | 0.9505 |
| CDC2 | 694849II [symbol] | −1.786 | −3.107 | −1.147 | −2.334 | −3.271 | −3.057 | −1.731 | 0.302 | −1.18 | −0.665 | −2.731 | −1.895 | −1.883 | −2.04 | −1.598 |
| CDC6 | 693742II [symbol] | 0.382 | −2.827 | −2.804 | −2.483 | −2.669 | −2.897 | −1.961 | −1.37 | −0.73 | −0.107 | −3.316 | −1.731 | −1.543 | 0.658 | −1.45 |
| CDCA1 | 693999II [symbol] | −1.957 | −0.275 | −0.346 | −2.503 | −1.061 | −3.157 | 0.497 | 0.759 | −0.98 | −0.988 | −2.677 | −0.691 | −0.553 | −1.984 | −0.293 |
| CDCA5 | 696764II [symbol] | −1.351 | −1.791 | −0.845 | −2.941 | −2.442 | −2.038 | −0.149 | −0.298 | −1.18 | 0.097 | −2.336 | −0.759 | −1.752 | −2.317 | −0.778 |
| CDCA7 | 686391II [symbol] | 1.048 | 1.718 | −1.07 | −1.101 | −0.761 | 0.34 | −0.034 | −2.561 | −0.04 | −0.221 | −0.725 | 0.299 | −0.002 | 0.454 | −2.281 |
| CDH1 | 693949II [symbol] | 0.988 | 3.655 | 0.5 | 0.769 | 2.139 | 1.053 | 0.532 | 1.402 | 0.28 | 0.586 | 1.408 | 0.698 | 2.098 | 0.365 | 1.085 |
| CDH3 | 689567II [symbol] | 0.1065 | −0.6695 | 0.3055 | −1.215 | −2.044 | 0.0915 | 2.283 | 2.532 | 1.66 | −0.7535 | −0.6575 | 1.535 | −0.0915 | 1.034 | 0.2975 |
| CDK2 | 690323II [symbol] | −0.0835 | 0.0915 | −0.5355 | −0.5385 | −0.7835 | 0.3585 | 0.6265 | 0.4155 | 0.02 | 1.05 | −0.8025 | 0.3965 | −0.9725 | −0.9685 | −1.081 |
| CDKN2A | 692881II [symbol] | 0.48 | −0.701 | 0.005 | −0.185 | 0.29 | −0.011 | 0.58 | 1.512 | −0.7 | −1.197 | 0.399 | 0.349 | 0.293 | 0.158 | −0.717 |
| CDKN2D | 682687II [symbol] | 0.105 | −0.672 | −0.796 | 0.977 | 0.475 | −0.015 | 2.584 | 1.656 | −0.48 | −0.715 | −0.272 | −0.37 | 0.662 | 0.706 | 0.764 |
| CDKN3 | 684517II [symbol] | 1.155 | 0.0225 | 0.6125 | −0.2165 | −0.5145 | −0.3395 | 1.094 | 1.792 | −0.5 | 2.701 | −0.2945 | −0.1895 | 1.179 | 0.8025 | 0.7235 |
| CDT1 | 686706II [symbol] | 0.9685 | 0.8445 | 0.9075 | −0.5445 | 0.4155 | −0.5295 | 1.168 | 1.949 | −1 | 2.745 | −0.7125 | 1.089 | 0.6985 | 0.0485 | 1.638 |
| CEBPG | 688002II [symbol] | −0.5215 | 0.8205 | 0.7785 | 0.4055 | 0.3205 | −0.5065 | 0.4745 | 0.4505 | −0.2 | −0.4875 | −0.0345 | −0.5875 | 0.0545 | −0.2155 | 0.6595 |
| CELSR1 | 686084II [symbol] | −0.4545 | 1.457 | −2.011 | 1.319 | 0.0355 | 0.9606 | −1.17 | −0.0005 | 1.36 | −0.0545 | 0.2825 | −1.277 | 0.1255 | −0.4705 | −1.464 |
| CENPA | 697624II [symbol] | −0.957 | −1.205 | −0.237 | −1.471 | −1.733 | −1.145 | 0.251 | 0.008 | −0.87 | 0.313 | −1.879 | −0.317 | −0.875 | −1.279 | −0.32 |
| CENPE | 683395II [symbol] | 0.6663 | 0.5683 | 1.579 | −0.4887 | 0.3323 | −0.8337 | 1.905 | 1.683 | −0.73 | 2.758 | −0.6307 | 1.366 | 0.6473 | 2.041 | 2.555 |
| CENPF | 691312II [symbol] | 0.668 | −0.207 | 1.9 | −0.087 | −1.298 | −1.14 | 2.718 | 2.703 | −0.69 | 0.786 | −0.835 | 1.774 | 1.058 | 0.661 | 2.108 |
| CENTG3 | 687661II [symbol] | 0.326 | −0.087 | −0.152 | 0.656 | 1.072 | 0.073 | 0.25 | −0.565 | 0.43 | −0.277 | −0.094 | 0.378 | −0.081 | 0.181 | −0.073 |
| CES2 | 897031II [symbol] | −0.717 | 0.314 | −1.005 | −0.565 | 1.121 | −0.119 | −0.309 | −1.038 | 0.17 | −1.105 | 0.139 | 0.227 | −0.16 | −0.853 | −1.119 |
| CETN3 | 692233II [symbol] | −1.564 | −0.647 | −1.615 | −0.99 | −0.782 | −0.77 | −1.059 | −0.493 | −0.13 | −0.065 | −0.886 | −1.672 | −0.526 | −1.568 | −0.917 |
| CGA | 693621II [symbol] | −2.334 | −3.042 | −2.787 | 3.033 | 2.423 | 2.196 | −2.713 | −2.271 | 0.01 | 2.446 | −2.728 | −3.023 | −2.611 | −3.22 | −3.636 |
| CGI-12 | 683023II [symbol] | −0.449 | 0.614 | 0.672 | −0.443 | −0.355 | 0.933 | 0.997 | 0.215 | 0.15 | −0.007 | 0 | 0.577 | 0.125 | 1.222 | 0.873 |
| CGI-45 | 693157II [symbol] | −1.749 | −0.053 | −1.223 | −1.314 | −1.344 | −1.647 | −1.658 | 0.415 | 0.03 | 0.038 | −1.349 | −1.632 | −1.056 | −1.749 | −0.402 |
| CGI-49 | 684734II [symbol] | −0.522 | 0.249 | 1.013 | −0.106 | −0.032 | −0.503 | 0.07 | 0.416 | 0.91 | 0.742 | −0.334 | 0.05 | 0.026 | 0.033 | 0.285 |
| CGN | 688407II [symbol] | −2.238 | 1.395 | 0.1225 | 0.6815 | 1.625 | 0.5405 | −1.313 | −0.5195 | 0.39 | −1.256 | 0.5595 | 0.2325 | 0.0745 | −0.8675 | −0.3585 |
| CGNL1 | 690560II [symbol] | 0.2305 | −1.182 | 0.4365 | 0.3375 | −1.853 | −0.3945 | −1.697 | 0.6215 | 0.74 | −3.081 | 0.5285 | −1.086 | 0.6455 | −1.532 | −0.0075 |
| CHAF1B | 682830II [symbol] | 0 | −0.386 | 0.44 | −0.03 | −0.434 | −0.379 | 1.21 | 1.38 | −0.67 | 1.6 | −0.343 | 1.556 | 0.423 | −0.384 | 1.82 |
| CHC1 | 682220II [symbol] | 1.047 | −0.205 | −1.126 | −1.552 | −0.528 | 0.22 | 1.709 | 0.565 | 0.01 | −0.413 | −0.213 | 1.141 | 0.271 | 0.6 | 0.45 |
| CHCHD2 | 681919II [symbol] | 0.0055 | −0.2435 | 0.3045 | 0.1155 | 0.3905 | −0.3385 | −0.0055 | −0.5105 | −0.15 | 0.6045 | −0.3695 | −0.6255 | 0.8955 | −0.1585 | 0.0565 |
| CHCHD5 | 686549II [symbol] | −0.0705 | 0.8725 | −0.6115 | 0.1055 | 0.7895 | 0.8765 | −0.7015 | −0.4735 | 0.03 | −0.2495 | 0.2245 | −0.5605 | 0.0175 | −0.2585 | −1.157 |
| CHI3L1 | 689913II [symbol] | 1.926 | −0.862 | 3.326 | −0.517 | −1.014 | −0.208 | 2.437 | 2.677 | 1.23 | −0.677 | 0.136 | 3.357 | −0.341 | −0.032 | 1.471 |
| CHI3L2 | 683767II [symbol] | −0.3245 | −1.912 | 3.966 | −1.331 | 1.666 | −0.3845 | 1.148 | 3.925 | −0.09 | −2.073 | −0.2675 | 4.971 | 0.5105 | −1.049 | 2.531 |
| CHKA | 689809II [symbol] | −0.0645 | −0.4815 | 2.037 | 0.0415 | −0.7155 | 0.1815 | 0.0155 | 0.0625 | 0.69 | −0.2345 | −1.129 | 0.8125 | −0.4045 | 0.3605 | −0.2005 |
| CHODL | 694845II [symbol] | 0.572 | −1.555 | 0.33 | −2.058 | −2.549 | −0.008 | −2.758 | −2.965 | 0.12 | 3.058 | 0.024 | −1.896 | −3.517 | −2.785 | −2.94 |
| CHRDL2 | 686365II [symbol] | 0 | 0.046 | 0.155 | −0.0925 | −0.084 | 0.747 | −0.356 | 1.666 | 0.1 | −0.018 | 0.592 | −0.017 | 0.094 | −0.532 | −0.003 |
| CHST2 | 691695II [symbol] | 0.0665 | 0.0925 | 0.1365 | −0.2855 | −0.0385 | 0.0185 | −0.1015 | 0.5735 | −0.24 | −0.2485 | 0.1995 | 0.0875 | −0.2895 | −0.2175 | 0.1185 |
| CHURC1 | 684314II [symbol] | −0.044 | 0.419 | −0.274 | 0.168 | 0.472 | −0.163 | −0.525 | 0.16 | −0.22 | 0.89 | 0.148 | −0.893 | −0.189 | −0.309 | −0.212 |
| CIR | 696941II [symbol] | 0.68 | 0.632 | 0.612 | 0.662 | 0.653 | 0.949 | 0.375 | −0.381 | 0.14 | 0.241 | 0.941 | 0.589 | 0.138 | 0.525 | 0.535 |
| CIRBP | 685402II [symbol] | −0.503 | 0.164 | −1.166 | 0.521 | 0.383 | 0.113 | −0.802 | −1.71 | 0.87 | −0.804 | 0.428 | −0.916 | 0.167 | −0.502 | −0.899 |
| CKLF | 688221II [symbol] | −0.1775 | 0.4685 | −1.151 | −0.3175 | 0.4355 | −0.9565 | 0.0945 | 0.9685 | −0.39 | −0.1145 | 0.2086 | −0.0555 | 0.6185 | −0.4545 | 0.2615 |
| CKLFSF6 | 681860II [symbol] | −0.407 | −0.021 | −0.846 | −0.179 | 1.593 | −0.64 | −0.606 | 0.446 | 0.26 | −0.567 | 0.116 | −1.092 | −0.368 | −0.468 | 0.417 |
| CKLFSF7 | 694344II [symbol] | 2.061 | 1.274 | −1.115 | −1.018 | 1.1 | 0.104 | 1.293 | 1.826 | 0.39 | −1.165 | 0 | −0.019 | 0.439 | −0.017 | 2.19 |
| CKMT1 | 692149II [symbol] | 1.597 | −0.531 | 0.382 | 1.92 | −0.742 | 0.378 | 1.791 | 0.739 | 0.05 | 1.694 | 0.342 | 0.05 | 0.889 | 1.432 | 1.22 |
| CKS1B | 696332II [symbol] | −1.804 | −1.471 | −0.752 | −2.062 | −2.013 | −1.37 | 0.518 | −0.135 | −0.31 | 0.388 | −1.766 | −0.41 | −0.773 | −1.216 | 0.204 |
| CKS2 | 691352II [symbol] | 0.283 | 0.051 | 1.526 | −0.3 | 0.408 | −0.13 | 1.56 | 2.48 | −1.26 | 1.762 | −0.739 | 0.194 | 0.051 | −0.457 | 1.159 |
| CL640 | 695106II [symbol] | −0.898 | −0.29 | −1.398 | −1.395 | −0.677 | −0.881 | 0.052 | 0.46 | −0.3 | −0.757 | −0.133 | −0.834 | −0.169 | −1.31 | 0.312 |
| CLDN23 | 683062II [symbol] | 0.194 | 0.1337 | 1.514 | −1.279 | −0.205 | −0.342 | 3.05 | 1.596 | 0.36 | 0.717 | 0.06 | 0.688 | −0.061 | −0.964 | 0.805 |
| CLDN3 | 686923II [symbol] | −0.5225 | 1.454 | 1.795 | −0.7775 | 0.1405 | 1.529 | −0.3765 | 0.3705 | −0.92 | −1.949 | −2.487 | 0.7845 | 0.0245 | −1.499 | 0.2695 |
| CLDN4 | 681712II [symbol] | −0.8355 | −0.1085 | 0.7985 | −0.0545 | 0.2535 | 0.5015 | −0.0735 | 0.7405 | −0.68 | 0.6885 | −1.434 | 0.6865 | −0.0475 | −0.0445 | 0.0065 |
| CLDN8 | 695858II [symbol] | 0.372 | −0.675 | 1.439 | 1.505 | −0.549 | −0.144 | 0.248 | 0.26 | 1.08 | 0.07 | 0.107 | 0.57 | −0.026 | 0.738 | 2.212 |
| CLGN | 691474II [symbol] | 1.772 | −1.737 | −1.607 | 3.193 | −0.1175 | 0.4255 | −1.071 | −0.6305 | 0.37 | −2.312 | −0.0316 | −1.713 | 0.6745 | −1.46 | −0.1445 |
| CLIC6 | 692133II [symbol] | −0.274 | 0.213 | 0.13 | 0.229 | 0.135 | 3.694 | −0.659 | 0.635 | 1.91 | 0.32 | 1.197 | −0.746 | −0.554 | −0.293 | 2.798 |
| CLNS1A | 693088II [symbol] | −1.5 | 0.836 | 0.725 | −1.05 | −0.507 | −0.743 | −0.995 | −0.417 | 0.03 | −0.681 | −0.043 | −0.423 | −1.702 | −0.522 | 0.025 |
| CLTC | 694429II [symbol] | −0.443 | 0.762 | −1.251 | −1.525 | −0.326 | −0.909 | −1.043 | 0.289 | −0.3 | −0.836 | −0.066 | −0.566 | 0.204 | −0.829 | −0.171 |
| CLU | 691706II [symbol] | 0.5765 | −0.8835 | 2.479 | 1.45 | −0.8845 | 0.0185 | −1.841 | −0.7245 | 0.48 | −1.901 | 0.6425 | 0.2215 | −0.4995 | −1.858 | −0.5135 |
| CMAS | 682207II [symbol] | −0.042 | 0.816 | 1.884 | 0.432 | 0.587 | −0.475 | −0.36 | 0.107 | −0.27 | 0.647 | −0.393 | 0.182 | 0.133 | 0.123 | 1.692 |
| CML66 | 682090II [symbol] | −0.25 | 1.121 | 1.07 | −0.48 | −0.608 | 0.065 | 1.25 | 0.93 | −0.52 | 0.78 | −0.442 | 0.477 | 0.552 | −0.195 | 1.26 |
| CNIH | 685679II [symbol] | 0.2875 | −0.8275 | 0.3385 | 0.2995 | −0.5575 | −0.7165 | −0.6055 | 0.2485 | −0.06 | 0.7075 | 0.1925 | −1.412 | 0.8565 | 1.392 | 0.5315 |
| CNOT10 | 688504II [symbol] | 0.043 | 0.028 | 1.157 | 0.225 | 0.27 | 0.004 | −0.027 | −0.053 | 0.01 | 0.377 | −0.88 | −0.704 | −0.507 | −0.3 | −0.239 |
| CNOT2 | 688501II [symbol] | 0.2955 | 0.1975 | −0.5125 | −0.0395 | 0.0215 | −0.0465 | −0.2315 | −0.2605 | 0.34 | −0.7435 | −0.0215 | 0.2255 | −0.1725 | −0.1505 | 0.4635 |
| CNOT7 | 687730II [symbol] | −0.3705 | −0.2185 | 0.9905 | 0.3145 | 0.1555 | −0.5776 | −0.0545 | −0.0325 | 0.26 | 0.4755 | 0.7565 | 0.0455 | 0.0325 | −0.8115 | −0.2525 |
| CNP | 684683II [symbol] | −0.369 | −1.178 | −0.722 | −0.642 | −0.464 | −0.396 | 0.099 | −1.289 | 0.14 | −1.055 | −1.389 | −0.388 | −1.195 | −0.886 | −0.992 |
| CNTNAP2 | 687862II [symbol] | 1.741 | 1.048 | −0.365 | −0.104 | 2.444 | 3.888 | −1.598 | −2.514 | 0.48 | 1.805 | −2.856 | −2.216 | 0.983 | −0.282 | −2.465 |
| COASY | 694546II [symbol] | −0.467 | 0.503 | −1.231 | −0.002 | 0.803 | 0.605 | 0.033 | 0.076 | 0.18 | −0.661 | −0.053 | 0.157 | −1.026 | −0.74 | −0.386 |
| COL11A2 | 698658II [symbol] | −0.481 | −0.11 | 0.64 | −0.569 | −0.059 | −0.066 | 0.503 | −0.276 | −0.01 | −0.449 | −0.187 | 1.724 | −0.353 | 0.056 | −0.487 |
| COL27A1 | 690790II [symbol] | −0.7165 | −1.859 | 2.106 | −1.74 | 0.1725 | −0.5675 | 1.567 | 0.5055 | 1.14 | 0.4345 | −0.3055 | 0.5675 | 0.0235 | −0.1905 | 0.6515 |
| COL4A5 | 695636II [symbol] | −0.436 | −0.875 | −0.269 | 0.987 | 0.192 | −2.484 | −0.636 | −0.526 | 1.91 | −2.373 | −0.208 | −1.233 | 0.429 | −1.113 | −1.684 |
| COL9A1 | 688215II [symbol] | −0.138 | −1.447 | 0.782 | −0.556 | −1.095 | −0.499 | −0.091 | 0.294 | 0.3 | 0.421 | −0.977 | 1.665 | 0.005 | 0.446 | 0.171 |
| COL9A3 | 694474II [symbol] | −2.927 | −0.477 | 2.932 | −3.638 | −1.171 | −3.019 | −0.748 | −2.447 | 0 | 2.387 | −2.969 | 1.369 | 3.463 | −3.021 | −2.294 |
| CDMMD [symbol] | 685920II [symbol] | −0.519 | 0.648 | −0.034 | −0.323 | 0.745 | −0.032 | 0.359 | 0.225 | 0.27 | −0.252 | −0.592 | 0.384 | −0.34 | −0.008 | −0.015 |
| COMMD5 | 683292II [symbol] | 0.2925 | 1.111 | 0.1295 | 0.0085 | −0.1365 | 0.9785 | 0.4225 | 0.6735 | −0.29 | 0.2965 | −0.0935 | 1.078 | 0.7155 | −0.7545 | −0.4355 |
| COPS7B | 686043II [symbol] | 0.195 | 0.254 | 0.348 | −0.044 | 0.168 | 0.08 | −0.224 | 0.107 | −0.08 | −0.266 | 0.078 | 0.262 | −0.199 | 0.023 | 0.266 |
| CORO6 | 691905II [symbol] | −0.3585 | −0.1175 | −0.3425 | −0.4055 | 0.0005 | −0.0005 | 0.4026 | −0.4125 | 0.36 | 0.1255 | 0.0455 | −0.3705 | −0.0665 | 0.3195 | −0.3805 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| CORT | 688125II [symbol] | 0.5285 | −0.0475 | −0.2555 | 0.1155 | 0.0285 | 0.4525 | 0.0305 | −0.4275 | −0.18 | −0.2525 | −0.2415 | 0.4495 | 0.2345 | −0.4225 | 0.3395 |
| COX17 | 698533II [symbol] | 0.474 | 1.017 | −0.639 | 0.867 | 1.187 | 0.43 | −0.049 | 0.385 | −0.59 | 0.511 | 0.056 | 0.427 | −0.12 | 0.2 | 0.511 |
| COX5A | 690776II [symbol] | 0.29 | 1.046 | 0.507 | −0.03 | 0.051 | −0.332 | −0.117 | 0.306 | −0.48 | 1.244 | −0.11 | −0.559 | 0.062 | 0.632 | 0.881 |
| COX5A | 693143II [symbol] | −0.678 | 0.14 | −0.429 | −1.007 | −0.683 | −1.043 | −1.153 | −0.737 | −0.45 | 0.29 | −0.722 | −1.328 | −0.863 | −0.26 | −0.073 |
| COX6A1 | 690654II [symbol] | −0.2305 | 0.7275 | −0.8495 | 0.0745 | −0.0545 | 0.6795 | −0.2785 | 0.1005 | −0.46 | −0.3245 | 0.3015 | 0.3455 | −0.2445 | −1.12 | 0.4505 |
| COX6C | 685569II [symbol] | −1.498 | 1.687 | −0.7865 | 2.7 | 3.523 | 3.64 | −0.4285 | −0.8995 | −0.24 | −1.725 | 2.648 | −0.3555 | 2.742 | −0.5525 | −0.7795 |
| COX7A2 | 697312II [symbol] | −0.207 | −0.583 | 0.172 | 0.012 | 0.086 | −0.518 | 0.042 | 0.043 | −0.44 | −0.684 | −0.416 | 0.338 | 0.216 | −0.338 | 0.205 |
| CP | 683747II [symbol] | 0.2635 | −1.411 | −0.0895 | −0.4235 | 0.7025 | 0.4965 | 0.3425 | 0.5955 | 0.97 | −0.4985 | −0.0445 | 0.4305 | 0.2595 | 0.4625 | 0.9185 |
| CPAMD8 | 690973II [symbol] | 0.098 | −1.218 | −1.804 | 4.213 | 2.702 | 0.238 | −0.733 | −0.744 | 1.37 | −0.388 | 0.214 | −0.768 | −0.985 | 1.907 | −1.008 |
| CPB1 | 697093II [symbol] | −1.077 | −0.133 | −0.413 | 0.673 | 2.946 | 0.983 | 0.229 | −0.285 | 0.66 | −2.449 | −0.613 | −0.225 | 1.882 | −1.457 | −1.706 |
| CPEB3 | 683341II [symbol] | 0.0105 | 0.2765 | −0.7825 | 0.8965 | 0.7705 | −0.1355 | −0.8095 | −0.8255 | 0.4 | −1.049 | 0.1875 | −1.302 | −0.9115 | 0.0005 | −0.1055 |
| CPEB4 | 681384II [symbol] | −1.711 | 0.244 | 1.798 | −1.124 | 0.599 | −0.069 | 1.228 | 0.113 | −0.71 | 0.061 | −1.255 | 0.659 | 0.637 | −1.25 | 1.446 |
| CPSF3 | 690199II [symbol] | 0.242 | 0.604 | 0.079 | 1.643 | 1.997 | 0.709 | 0.717 | −0.201 | 0.04 | 1.266 | 0.86 | −0.242 | −0.121 | 0.3 | 1.4 |
| CRABP1 | 694973II [symbol] | −0.068 | −0.565 | 2.097 | −0.407 | −0.387 | −0.12 | −0.261 | 1.168 | −0.05 | −0.649 | 0.089 | 1.958 | 0.483 | −0.586 | 0.293 |
| CREB3L4 | 690183II [symbol] | −2.32 | −1.919 | −1.48 | −2.33 | −1.636 | −1.474 | −1.14 | −1.56 | 0.52 | −0.36 | −1.175 | −0.486 | −1.189 | −1.908 | −0.73 |
| CRIP1 | 686560II [symbol] | 0.999 | −0.113 | −0.51 | 0.426 | 0.079 | 0.774 | −2.421 | −1.427 | 0.22 | −0.354 | 0.704 | −1.64 | −0.786 | −0.412 | −2.455 |
| CRYBB2 | 683857II [symbol] | −0.094 | 0.812 | −0.013 | −0.318 | 0.064 | 0.826 | 1.198 | −0.039 | −0.46 | −0.422 | 0.244 | −0.02 | −0.41 | −0.277 | 0.37 |
| CRYL1 | 692598II [symbol] | 0.064 | −0.259 | −1.416 | −0.915 | 0.944 | −0.253 | −0.768 | −0.562 | −0.03 | −0.668 | 0.152 | −1.043 | −0.559 | 0.277 | −0.3 |
| CRYZ | 683831II [symbol] | −1.439 | 0.0005 | −0.5195 | −0.1265 | −0.1375 | 1.092 | −0.0915 | 0.4175 | 0.41 | −0.6205 | 0.0395 | 0.2595 | −0.2795 | −0.7985 | 1.175 |
| CSDA | 681102II [symbol] | 0.0585 | −0.0315 | 0.3015 | −0.4235 | −0.3135 | −0.0465 | 0.6365 | 0.2485 | 0.41 | 1.135 | −0.4915 | 0.8245 | 0.0455 | −0.1755 | −0.0655 |
| CSE1L | 693508II [symbol] | −1.468 | −1.295 | −0.32 | −1.551 | −1.273 | −2.315 | −0.459 | −0.797 | −0.4 | 0.934 | −0.146 | −1.668 | −1.147 | −1.397 | −0.777 |
| CSK | 693320II [symbol] | 1.99 | 0.169 | 0.099 | 0.182 | 0.606 | 0.143 | 0.135 | 0.034 | −0.66 | 0.354 | −0.008 | 0.582 | 0.189 | 0.303 | 1.215 |
| CSRP1 | 681588II [symbol] | 0.405 | −0.052 | −0.895 | 0.289 | −0.134 | −0.842 | −0.308 | 0.294 | 0.88 | 0.223 | −0.417 | 0.458 | 0.663 | 0.551 | −0.334 |
| CSRP2 | 691158II [symbol] | 0.0765 | −0.4495 | 2.209 | −0.5815 | −0.8735 | 0.5525 | 1.558 | 2.914 | 0.49 | 1.44 | −0.0855 | 0.8725 | −0.9775 | 0.2135 | 0.9395 |
| CST6 | 688329II [symbol] | 1.422 | 0.336 | 0.238 | −1.245 | −1.761 | 2.077 | −0.558 | −1.708 | 0.35 | 0.599 | −1.562 | −0.869 | −0.188 | 0.861 | −1.19 |
| CSTB | 688630II [symbol] | 0.9905 | −0.4045 | −0.1425 | −0.0625 | −0.4465 | 0.4115 | 0.2285 | 1.738 | −0.12 | 0.3925 | 0.2445 | 1.244 | 0.7865 | −0.3405 | 0.6285 |
| CTAG1B | 685110II [symbol] | −0.193 | 0.302 | 0.345 | −0.18 | 0.617 | 0.296 | −0.628 | −0.921 | 0.24 | −0.718 | −0.299 | 0.117 | 0.158 | 0.408 | −0.675 |
| CTBP2 | 690880II [symbol] | −0.139 | −0.551 | −0.079 | 0.045 | 0.085 | 0.012 | −0.147 | −0.523 | 0.13 | −0.246 | −0.055 | −0.041 | −0.441 | −0.374 | 0.459 |
| CTNNA1 | 682257II [symbol] | 0.55 | −0.309 | −3.171 | 0.102 | −0.856 | 0.542 | 0.025 | −3.295 | 0.24 | 0.219 | −0.054 | −0.361 | 1.068 | 0.461 | −0.137 |
| CTNNAL [symbol] | 683652II [symbol] | −1.421 | −0.3585 | −1.112 | −0.3355 | 0.9045 | 0.0635 | 1.134 | 2.142 | 0.99 | 2.063 | −0.2805 | 0.8715 | 0.2395 | −0.8305 | −0.4505 |
| CTNND2 | 692813II [symbol] | −0.16 | 0.878 | −0.494 | 0.047 | −0.438 | 0.266 | 0.244 | 0.138 | 0.89 | −1.246 | 0.826 | 0.332 | 0.544 | −0.211 | −0.352 |
| CTPS | 688832II [symbol] | 0.641 | 0.273 | −0.845 | −0.32 | 0.541 | −0.692 | 0.658 | −0.413 | −0.15 | −0.821 | 0.067 | −0.388 | 0.12 | 0.174 | 1.428 |
| CTSL2 | 696809II [symbol] | −1.342 | −2.125 | −0.547 | −3.147 | −2.725 | −3.572 | −0.627 | 0.409 | −0.77 | 3.572 | −2.468 | 0.661 | −2.239 | −3.192 | −0.241 |
| CTSS | 688045II [symbol] | 2.02 | −0.4505 | −1.272 | −0.2075 | 0.3565 | −0.0855 | −0.3655 | 0.5505 | −0.96 | −0.2865 | 0.7855 | 0.4135 | 0.0765 | −0.0765 | 1.747 |
| CTTN | 690610II [symbol] | −0.009 | −0.118 | 0.178 | −0.381 | −0.2165 | −0.847 | −0.63 | 0.261 | −0.35 | 0.76 | −0.501 | 0.388 | 2.463 | 0.468 | 0.449 |
| CX3CL1 | 762475II [symbol] | 0.063 | 2 | 3.532 | 0.862 | 3.14 | 1.93 | 1.865 | 3.454 | 0.67 | −0.855 | 1.02 | 2.16 | 1.79 | 1.24 | 4.065 |
| CXCL9 | 694763II [symbol] | 5.713 | 1.064 | 1.163 | 2.301 | 3.785 | 1.364 | 6.162 | 1.358 | −1.61 | 0.801 | 4.173 | 3.489 | 4.958 | 4.282 | 6.083 |
| CXorf39 | 682198II [symbol] | −0.3965 | 1.325 | 0.8595 | −0.2925 | 0.0705 | −0.0395 | 0.7335 | 0.7085 | −0.59 | 0.0395 | −0.0455 | −0.4375 | −0.9825 | 0.4945 | 0.3945 |
| CXXC4 | 684240II [symbol] | 0.2825 | −1.43 | −2.899 | 1.284 | 0.9305 | −2.872 | −0.4655 | −2.015 | 0.69 | −3.124 | 0.4285 | −2.239 | −2.986 | −0.7605 | 0.0145 |
| CYB5 | 690446II [symbol] | 0.373 | −1.299 | −0.312 | 0.872 | 1.354 | 0.696 | −1.725 | −1.637 | 0.47 | −1.635 | 0.869 | −1.97 | −0.641 | 0.823 | −0.809 |
| CYB561 | 683867II [symbol] | 1.093 | 1.675 | −1.202 | 0.543 | 0.025 | 0.548 | −0.887 | −0.679 | −0.53 | −0.572 | −0.248 | −0.276 | 0.665 | −0.382 | −1.901 |
| CYC1 | 683057II [symbol] | 1.469 | −0.3725 | −2.332 | −1.394 | 0.1405 | −0.0925 | 0.1455 | −1.021 | −0.72 | −1.555 | 0.3275 | −0.9825 | 0.7295 | 0.1105 | 1.134 |
| CYHR1 | 697209II [symbol] | 0.585 | 2.074 | 1.46 | 1.168 | 1.892 | 2.667 | 1.815 | 0.914 | −0.04 | 0.192 | 0.611 | 2.07 | 0.324 | 0.569 | −0.282 |
| CYP2A13 | 689236II [symbol] | 0.262 | −0.108 | −0.221 | 3.221 | 2.868 | 0.185 | 0.455 | −0.258 | −0.22 | 0.031 | −0.314 | −0.051 | −0.085 | −0.19 | −0.609 |
| CYP4X1 | 681940II [symbol] | −0.3317 | −1.621 | 0.7853 | 3.181 | −0.8897 | 1.624 | −1.034 | −0.2017 | 1.65 | −1.391 | 0.7433 | −1.532 | −0.2977 | 1.815 | −1.25 |
| D15Wsu7 [symbol] | 684516II [symbol] | 2.312 | 1.687 | −0.81 | −0.046 | −0.678 | −0.175 | −0.335 | −0.988 | 0.27 | −0.093 | 3.062 | −0.853 | 2.702 | −0.29 | −0.769 |
| DACH1 | 692995II [symbol] | 0.0635 | 0.0235 | −0.2485 | 0.4775 | −0.7655 | −0.3825 | −0.2595 | −0.2405 | 1.07 | 1.287 | −0.1315 | −1.28 | 0.5935 | −0.1575 | −0.0235 |
| DACH1 | 694269II [symbol] | 0.742 | 1.926 | 0.127 | 3.311 | 2.563 | 0.233 | 0.077 | 0.407 | 0.33 | 0.0416 | 1.635 | 0.201 | 1.814 | 0.214 | −0.019 |
| DACH1 | 697247II [symbol] | 1.002 | 2.13 | 0.012 | 3.591 | 2.412 | 0.196 | −0.414 | −0.064 | 1.25 | −0.235 | 2.863 | 0.101 | 1.968 | −0.31 | −0.43 |
| DAP3 | 689542II [symbol] | −0.242 | 1.535 | 0.022 | 0.934 | 1.757 | 0.54 | 0.658 | −0.295 | 0.03 | −0.472 | 0.23 | 1.233 | 0.673 | 0.891 | −0.037 |
| DAPP1 | 681676II [symbol] | −1.281 | −1.369 | 0.265 | −1.21 | −1.155 | −1.023 | −0.8 | −0.493 | −0.87 | −0.704 | −1.651 | −0.861 | −0.885 | −1.236 | −0.336 |
| DAZAP2 | 686305II [symbol] | −0.3725 | 0.6865 | −1.197 | −0.2115 | −0.3995 | 0.6835 | −0.5435 | −0.8115 | 0.19 | −1.96 | 0.8305 | −0.3475 | −0.8615 | −0.0895 | −0.2455 |
| DBI | 687444II [symbol] | 1.022 | 0.8265 | −0.1415 | −0.2735 | −0.4095 | −0.2395 | 0.6975 | 1.529 | −0.12 | −0.2195 | 0.3415 | 1.349 | −1.087 | −0.0095 | −0.6545 |
| DBN1 | 685238II [symbol] | 0.713 | 0.131 | 0.768 | −0.25 | −1.337 | −0.25 | 0.574 | 0.245 | 0.04 | −0.715 | −0.312 | −0.322 | 0.575 | 0.334 | 0.233 |
| DC13 | 693725II [symbol] | −1.52 | −0.667 | −1.31 | −1.817 | −1.486 | −1.319 | −0.33 | 0.411 | −0.21 | −0.656 | −1.534 | −0.537 | −0.352 | −1.502 | −0.183 |
| DCPS | 683983II [symbol] | 0.208 | 0.312 | 0.868 | −0.08 | 0.508 | 0.079 | 0.097 | −0.007 | −0.49 | 0.192 | −0.595 | 0.501 | 0.037 | −0.228 | 0.094 |
| DDHD2 | 683946II [symbol] | −0.3635 | 0.4915 | 0.5875 | 0.4105 | 0.4645 | 0.2425 | 0.3115 | 0.1085 | −0.09 | −0.0765 | −0.1775 | 0.3855 | 0.3035 | 0.9685 | −0.0645 |
| DDX17 | 694961II [symbol] | −0.325 | −0.152 | 1.689 | 0.625 | 0.323 | −0.052 | −0.496 | −0.103 | −0.3 | 0.212 | −0.67 | −0.528 | −0.158 | 0.531 | 0.299 |
| DDX39 | 681363II [symbol] | 0.3795 | 0.1245 | 0.6755 | −0.2315 | −0.3985 | 0.2135 | 1.043 | −0.0755 | −0.4 | 0.6765 | −0.3185 | −0.0325 | 0.3515 | 0.0485 | 0.2765 |
| DDX48 | 696985II [symbol] | −0.77 | 0.217 | −0.657 | −1.218 | −1.439 | −0.185 | −0.698 | 0.188 | −0.04 | −0.123 | −0.727 | −0.571 | −0.067 | −0.713 | −0.41 |
| DDX58 | 691620II [symbol] | −0.88 | −0.195 | −1.431 | −1.222 | 0.974 | −0.515 | 0.999 | 0.032 | 0.15 | −1.087 | 0.881 | −1.454 | 0.105 | 0.65 | 0.25 |
| DEF6 | 682749II [symbol] | 0.068 | −0.654 | −0.854 | −0.546 | −0.384 | −0.253 | 0.67 | −0.356 | 0.27 | −0.704 | 0.728 | −0.026 | 0.598 | −0.589 | 0.346 |
| DEK | 688515II [symbol] | −0.004 | −0.393 | −0.389 | 0.836 | −1 | −0.197 | 1.337 | 1.278 | 0.05 | 0.789 | −0.08 | 0.608 | 1.166 | 0.318 | 0.498 |
| DHCR7 | 694109II [symbol] | −0.0975 | −0.5875 | 0.9905 | 1.273 | 0.0155 | 0.4855 | 0.3605 | −0.0565 | −0.8 | −0.0375 | −0.3605 | −0.7285 | 0.1295 | 0.3945 | −0.1065 |
| DHRS7 | 682438II [symbol] | −0.071 | −0.202 | −0.258 | 0.97 | 0.773 | −0.244 | 0.099 | 0.38 | −0.21 | −0.587 | 1.157 | −1.057 | 1.08 | −0.001 | 0.409 |
| DHX57 | 686263II [symbol] | −0.242 | 0.11 | −0.203 | 0.018 | 0.003 | 0.697 | 0.058 | 0.208 | −0.17 | −1.484 | −0.079 | −0.862 | −0.284 | 0.065 | −0.303 |
| DIRC1 | 691337II [symbol] | −0.295 | −1.385 | −1.83 | −0.473 | 0.352 | 0.553 | −1.124 | 0.306 | 0.68 | −0.348 | 1.016 | 0.15 | −0.17 | 0.333 | −0.317 |
| dJ222E13 [symbol] | 689427II [symbol] | 2.96 | 0.898 | −1.95 | −0.87 | −0.315 | 3.64 | −1.876 | −0.588 | 2.64 | 0.263 | 1.979 | −0.504 | −1.552 | 0.595 | −2.129 |
| DKFZP43 [symbol] | 689930II [symbol] | −0.178 | 0.092 | −0.769 | 0.071 | 0.775 | −0.006 | 0.227 | −0.477 | 0.63 | −0.75 | −0.756 | 0.956 | −0.995 | −0.376 | −0.167 |
| DKFZp43 [symbol] | 689808II [symbol] | −0.309 | −0.658 | 1.825 | −0.203 | −0.869 | 0.111 | 1.576 | 2.045 | 0.16 | −1.283 | −0.056 | 1.015 | −0.767 | 0.715 | 2.025 |
| DKFZP43 [symbol] | 681071II [symbol] | 1.449 | 0.4035 | −0.8765 | −0.0085 | 0.2755 | 1.024 | 0.0085 | −0.2685 | −0.51 | 0.8845 | −0.5755 | 0.4935 | −0.2795 | 1.138 | 0.8575 |
| DKFZp43 [symbol] | 683495II [symbol] | 0.4705 | −0.4625 | 1.77 | −0.5325 | −0.1005 | 0.1505 | 1.03 | 0.6235 | −0.51 | −0.0595 | 0.3245 | 0.3295 | 0.6375 | 0.0025 | 1.376 |
| DKFZP56 [symbol] | 684976II [symbol] | 0.057 | −1.542 | 0.799 | −0.846 | −1.208 | −1.296 | 0.734 | 0.885 | −0.18 | −0.552 | −0.483 | 0.593 | −0.659 | −0.973 | 1.26 |
| DKFZp56 [symbol] | 697724II [symbol] | 0.254 | 1.156 | −0.824 | 0.475 | 0.546 | 0.184 | −0.226 | −0.216 | −0.21 | −0.141 | 0.631 | 0.164 | −0.202 | 0.353 | −0.165 |
| DKFZP56 [symbol] | 683868II [symbol] | 1.681 | 0.686 | −0.817 | −0.16 | 0.626 | −0.281 | −0.167 | 0.425 | 0.02 | 0.321 | −0.355 | 0.77 | 0.216 | 2.636 | 0.34 |
| DKFZP56 [symbol] | 688875II [symbol] | −0.4905 | 1.038 | 0.7275 | −0.4055 | −0.0195 | 1.096 | 1.173 | 0.7635 | −0.23 | 0.8315 | −0.3275 | 0.9075 | 0.8345 | −0.4505 | 0.8405 |
| DKFZP56 [symbol] | 682674II [symbol] | −0.367 | 0.963 | −0.682 | −0.013 | 0.236 | 0.014 | −0.245 | 0.148 | −0.07 | 0.441 | 0.576 | −0.659 | 0.011 | 0.171 | −0.29 |
| DKFZp58 [symbol] | 695204II [symbol] | −0.935 | −1.417 | 0.315 | −1.004 | −1.512 | −1.2 | −1.764 | −0.637 | −0.15 | −0.035 | −1.125 | −1.521 | −1.459 | −1.218 | −0.41 |
| DKFZp76 [symbol] | 681865II [symbol] | −0.1577 | 1.551 | −1.052 | 0.3933 | 1.047 | 0.1953 | 0.1553 | −0.8487 | 1.81 | −0.7277 | 0.2353 | 0.0963 | −0.0858 | −0.3597 | −1.025 |
| DLK1 | 685295II [symbol] | −0.125 | −0.452 | 3.323 | 0.043 | −0.387 | 0.151 | −0.446 | −0.498 | 1.18 | 0.284 | −0.427 | 0.007 | −1.071 | −0.307 | −0.857 |
| DLX5 | 682457II [symbol] | 2.838 | −0.321 | −4.3 | 1.018 | −1.116 | −1.771 | −4.617 | −3.72 | 0.09 | −4.282 | 0.713 | −3.312 | 0.535 | 2.988 | −4.171 |
| DNAJA4 | 681226II [symbol] | 0.458 | 2.584 | −0.638 | 0.632 | 0.124 | 0.139 | −0.74 | 0.042 | 0.23 | −2.497 | −0.573 | −0.866 | 0.257 | 0.328 | −0.405 |
| DNAJC1 | 692749II [symbol] | 1.517 | 1.627 | 0.083 | 1.521 | 1.413 | 1.289 | 0.041 | −0.262 | −0.28 | 0.605 | 0.332 | −0.299 | 1.491 | 0.755 | 0.57 |
| DNAJC12 | 696427II [symbol] | −0.483 | 0.603 | −2.422 | 3.074 | 3.602 | 2.264 | −3.661 | −1.687 | 1.98 | −2.463 | 2.03 | −2.403 | −0.18 | −2.071 | −2.767 |
| DNAJD1 | 690353II [symbol] | 1.077 | 0.0885 | 0.1855 | −0.5325 | 0.1765 | 0.3395 | 0.0415 | 0.2085 | −0.3 | 1.834 | −0.4276 | −0.2595 | −0.2735 | −0.3195 | 0.6505 |
| DNALI1 | 695070II [symbol] | −0.165 | 4.309 | −0.962 | 4.42 | 4.481 | 4.176 | 0.126 | −0.886 | 1.07 | 1.85 | 3.148 | −0.552 | 4.849 | −0.288 | −0.859 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| DNMT1 | 681126II [symbol] | 0.5797 | −0.7723 | −0.8023 | −0.2363 | −0.1923 | 0.2157 | −0.4693 | −0.7463 | 0.15 | −1.135 | 0.5007 | 0.3227 | 0.0188 | −0.2543 | 0.0698 |
| DQX1 | 697175II [symbol] | 0.798 | 0.609 | −1.166 | −1.009 | 0.307 | −0.8 | −0.876 | −0.688 | −0.21 | −0.741 | 0.215 | −0.534 | 1.044 | −0.596 | 0 |
| DSCR1 | 690367II [symbol] | 0.004 | −1.3 | −0.711 | −1.297 | −1.267 | 0.528 | 0.459 | 1.66 | 0.76 | 0 | 0.278 | 1.54 | −0.977 | −0.519 | 0.369 |
| DSCR1L2 | 685798II [symbol] | −0.19 | 0.658 | −0.745 | 0.205 | −0.045 | 0.023 | 0.931 | 1.227 | −0.35 | −1.239 | 0.097 | 0.843 | −0.225 | −0.216 | 0.108 |
| DSP | 695661II [symbol] | −1.628 | −1.03 | −2.021 | 0.075 | 0.799 | −0.59 | −2.887 | −2.723 | 0.92 | −4.809 | 0.292 | −3.213 | −0.098 | −0.622 | −2.092 |
| DTYMK | 692933II [symbol] | −0.975 | −0.965 | 0.021 | −0.997 | −1.908 | −1.06 | −0.203 | 0.424 | −0.11 | 0.193 | −1.204 | −0.766 | −0.817 | −0.994 | −0.466 |
| DUSP23 | 691997II [symbol] | −0.9345 | 0.1045 | −0.1475 | 0.0245 | 0.0615 | 0.4645 | 0.2215 | −0.0785 | 0.51 | −1.682 | −0.4295 | 0.5835 | 0.0005 | −0.6025 | −0.4215 |
| DUSP4 | 693976II [symbol] | 1.052 | −0.043 | −0.159 | 1.77 | 0.051 | 1.31 | −0.447 | −0.266 | 0 | −0.4 | 1.285 | −0.165 | 1.305 | 1.356 | −0.18 |
| DUSP6 | 693200II [symbol] | 1.502 | −0.909 | −1.932 | 0.141 | −0.909 | −0.346 | −1.056 | −1.885 | 1.76 | −1.596 | −0.495 | −1.874 | 1.263 | 0.744 | −0.991 |
| DVL1 | 682613II [symbol] | −0.281 | 0.15 | −0.412 | 0.404 | 0.753 | 0.568 | −0.236 | −0.022 | 0.33 | −0.258 | 0.221 | 0.802 | 0.421 | −0.076 | −0.744 |
| DXYS155 [symbol] | 694208II [symbol] | 1.101 | 0.401 | −0.089 | −0.148 | 0.511 | 0.241 | 0.427 | −0.102 | −0.09 | 0.085 | 0.231 | 0.335 | 0.043 | 0.277 | 0.875 |
| DYRK4 | 697043II [symbol] | 0.785 | 0.816 | 2.159 | 0.097 | 0.895 | 0.337 | 0.485 | 0.894 | −0.11 | −0.6 | 0.104 | 0.94 | 0.167 | 1.123 | 0.728 |
| [symbol] 2 | 695983II [symbol] | −0.689 | −0.157 | −0.391 | −1.13 | −1.547 | −0.088 | 0.606 | 0.063 | −0.43 | −0.178 | −1.246 | −0.321 | 0.308 | −0.832 | 0.056 |
| EAF2 | 690585II [symbol] | −0.312 | −0.383 | 0.438 | −0.114 | 0.504 | 0.738 | −0.349 | −1.683 | −0.36 | −0.12 | 0.551 | −0.558 | 0.039 | −0.26 | −0.008 |
| EAP30 | 695304II [symbol] | −0.74 | 0.494 | −0.673 | −0.645 | −0.522 | −0.27 | −0.837 | −0.234 | −0.09 | −0.317 | −0.268 | −0.254 | −0.165 | 0.277 | −0.574 |
| ECHDC1 | 695068II [symbol] | −1.128 | −0.267 | −0.814 | −1.093 | 0.054 | −0.22 | −1.356 | −0.047 | 0.46 | −0.94 | −0.415 | −0.89 | −1.946 | −1.695 | 0.504 |
| ECHDC3 | 693484II [symbol] | 0.01 | 1.913 | 2.217 | −0.817 | 2.533 | 1.978 | 1.16 | 1.333 | 0.02 | 2.079 | 1.258 | 1.41 | 0.938 | 0.8 | 1.285 |
| EEF1A2 | 696775II [symbol] | −0.617 | 1.335 | −1.099 | −2.08 | −0.719 | −2.007 | −2.374 | −2.674 | −0.56 | −0.131 | −1.213 | −2.418 | −0.927 | −0.449 | −2.553 |
| EFHC1 | 694066II [symbol] | −0.918 | 0.121 | −0.502 | 0.526 | 0.885 | 0.556 | −0.592 | −1.351 | 1.33 | −1.541 | 0.69 | −0.476 | 0.431 | −0.515 | −0.387 |
| EFNA4 | 687429II [symbol] | 2.2 | 0.661 | 0.323 | 1.81 | 1.016 | 2.191 | 0.2 | 1.463 | −0.13 | −1.041 | 2.212 | 0.744 | 1.404 | 2.102 | 2.207 |
| EF5 | 685583II [symbol] | 0.5675 | 1.18 | −0.4105 | −0.6195 | −1.177 | 0.9405 | −0.0575 | 1.06 | 0.43 | 0.5945 | −1.267 | 1.284 | 0.4705 | −0.1445 | −0.5725 |
| EIF2C2 | 696794II [symbol] | 0.076 | 0.754 | 0.343 | −0.551 | −0.201 | 0.028 | 0.801 | 1.624 | −0.73 | 0.546 | −0.6 | 0.659 | −0.107 | −0.671 | −1.199 |
| EIF2C4 | 689728II [symbol] | 0.6255 | 0.0405 | 0.9055 | −0.4345 | −0.0955 | −0.6985 | 1.466 | 2.576 | 0.39 | 1.536 | −1.157 | 1.032 | 0.7225 | −0.0405 | 1.056 |
| EIF4EBP1 | 683217II [symbol] | −0.181 | −0.895 | 2.648 | −1.18 | −0.914 | −0.83 | 0.009 | −0.151 | −0.3 | −0.525 | −0.001 | 0.734 | 0.019 | −1.411 | −1.297 |
| EIF4G1 | 692045II [symbol] | 0.8795 | 0.2575 | −0.1195 | 0.5285 | 0.4125 | 0.0055 | 0.5815 | −1.435 | −0.16 | −0.0055 | −0.0735 | −0.2605 | −0.6085 | −0.2515 | −0.0075 |
| EIF4G3 | 683768II [symbol] | −0.258 | −0.58 | −0.957 | 0.523 | 0.182 | −0.154 | 0.2 | −0.678 | 0.33 | −0.458 | −0.392 | −0.564 | 0.383 | 0.363 | −0.58 |
| ELAC1 | 695486II [symbol] | −1.605 | −0.947 | 0.64 | −1.001 | −0.084 | −0.16 | 0.46 | −1.134 | 0.19 | 0.032 | −0.453 | 0.52 | −1.498 | −1.382 | 1.252 |
| ELL2 | 684066II [symbol] | 0.8535 | −0.9295 | −1.933 | 1.464 | −1.415 | 0.0575 | −0.8225 | −0.1875 | −0.61 | −0.5635 | −0.2915 | −1.478 | 1.116 | 1.309 | 1.359 |
| ELL3 | 695507II [symbol] | −0.1218 | 1.188 | 0.5092 | −0.2708 | 0.1952 | 0.6332 | 1.192 | 1.133 | −0.16 | 0.9062 | −0.1058 | 0.8272 | 0.3332 | −0.5918 | 0.1782 |
| ELOVL5 | 694593II [symbol] | −0.832 | −2.216 | −0.815 | 0.978 | −1.656 | 0.659 | −1.012 | −1.051 | 1 | −0.322 | 0.923 | −1.516 | 1.599 | 0.094 | −0.953 |
| EN1 | 689084II [symbol] | 1.265 | −0.0465 | 0.6955 | −0.5135 | −0.0655 | 0.1425 | 4.171 | 3.459 | −0.67 | 0.6085 | −0.3305 | 3.734 | −0.2635 | −0.0095 | 2.154 |
| ENAH | 682255II [symbol] | 0.186 | 0.019 | 0.29 | −0.42 | −0.459 | −1.811 | −0.119 | 1.236 | −0.19 | −1.222 | −0.572 | 0.487 | −0.499 | 0.822 | 0.238 |
| EPHX2 | 683128II [symbol] | −1.052 | −0.91 | 0.505 | 0.997 | 0.925 | 0.18 | −0.824 | −0.417 | 0.49 | −1.301 | −1.885 | −0.966 | −0.928 | −0.315 | 0.933 |
| EPLIN | 687610II [symbol] | −0.3575 | 0.7315 | −2.213 | −0.1725 | −0.7255 | −0.3315 | −1.236 | −1.434 | 0.72 | 0.3485 | 0.7655 | −2.055 | −0.6365 | 0.6145 | −1.253 |
| EPN3 | 691782II [symbol] | −0.183 | 2.598 | −1.203 | 0.71 | 1.232 | −0.423 | −1.214 | 0.152 | −0.02 | −0.161 | 0.05 | 0.516 | −0.157 | −0.029 | −0.762 |
| EPOR | 684991II [symbol] | 0.0025 | −0.0435 | −1.081 | 1.054 | 0.9365 | 1.906 | −0.1585 | −0.7305 | 1.24 | −0.0675 | 0.3165 | −0.7775 | −0.0025 | −0.7995 | −0.1155 |
| ERBB2 | 688427II [symbol] | 3.101 | 0.148 | −0.733 | 0.78 | 0.252 | 0.36 | −0.037 | −0.32 | 0.96 | −0.773 | 0.018 | −0.018 | −0.978 | 3.955 | −1.586 |
| ERP70 | 696269II [symbol] | 0.085 | −1.413 | −0.323 | −1.04 | −0.638 | −1.127 | 0.145 | −0.514 | −0.06 | 0.629 | −1.061 | 0.241 | −0.179 | 0.71 | −0.695 |
| ESR1 | 765041II [symbol] | 2.521 | 2.922 | 2.427 | 0.86 | −2.369 | 3.048 | 0.089 | 1.733 | 2.497 | 1.172 | 0.678 | 1.211 | 1.518 | 1.387 | 1.799 |
| ETFA | 693087II [symbol] | −0.261 | −1.922 | −2.058 | −2.397 | −2.046 | −3.114 | −1.414 | −2.115 | −0.05 | −0.156 | −1.907 | −3.324 | −1.316 | −1.352 | −1.825 |
| ETV6 | 697286II [symbol] | 1.328 | 1.156 | 2.601 | 1.176 | 0.473 | 2.265 | 0.876 | 1.897 | −0.34 | 0.691 | 0.899 | 2.268 | 0.899 | 1.803 | 2.568 |
| EVER1 | 688220II [symbol] | 0.9895 | 0.3085 | 0.3935 | −0.2055 | −0.8265 | −0.3875 | 0.2865 | −0.2225 | −0.85 | −0.0825 | −0.6205 | 0.7775 | 1.404 | 0.3695 | 0.2215 |
| EXO1 | 689742II [symbol] | −0.424 | 1.481 | −1.218 | −0.454 | −0.525 | 1.012 | −0.005 | −0.205 | −0.8 | −0.411 | 0.737 | −0.464 | −0.079 | −0.286 | −0.145 |
| EXOSC9 | 691275II [symbol] | 0.229 | 0.064 | −1.058 | 0.077 | 0.113 | −0.319 | −0.175 | −0.403 | −0.11 | 0.162 | −0.25 | −0.174 | 0.334 | −1.085 | 0.241 |
| EXT2 | 693031II [symbol] | 0.134 | 0.0032 | 0.548 | −0.344 | 0.485 | 0.026 | −0.05 | 1.053 | 0.33 | 0.923 | 0.526 | 0.125 | −0.423 | 0.537 | 0.165 |
| EZH2 | 689533II [symbol] | 0.549 | −0.296 | 0.168 | −0.678 | −0.578 | −0.545 | 0.434 | −0.146 | −0.52 | −0.443 | −0.361 | −0.122 | 0.352 | 0.183 | 0.606 |
| F11R | 684061II [symbol] | 1.289 | 0.891 | 0.113 | 0.619 | 0.714 | 0.935 | −0.008 | 0.01 | 0.12 | 0.407 | 1.006 | 0.636 | 0.173 | 0.349 | 0.216 |
| F9 | 682839II [symbol] | −1.008 | 0.025 | −0.866 | 0.609 | −0.676 | −0.116 | −0.272 | −0.387 | 0.13 | −2.114 | 0.985 | −1.117 | −0.031 | −1.35 | −0.681 |
| FA2H | 688063II [symbol] | 2.98 | 1.843 | −1.026 | 0.689 | −0.069 | −1.359 | 0.543 | −0.759 | −0.09 | −1.876 | 0.614 | −1.782 | −0.383 | 0.069 | −0.58 |
| FABP7 | 694559II [symbol] | −1.956 | −1.792 | 1.785 | −1.78 | −1.187 | −1.915 | −2.005 | 3.405 | 1.35 | −1.852 | −2.609 | 4.104 | −1.852 | −1.832 | 1.657 |
| FADD | 686191II [symbol] | −0.199 | 0.394 | −0.099 | −0.156 | 0.214 | −0.081 | −0.203 | −1.077 | −0.06 | 0.29 | 0.368 | 0.423 | 2.643 | −0.205 | −0.004 |
| FAHD1 | 688442II [symbol] | 0.503 | 0.775 | −0.274 | 0.414 | 0.869 | 2.045 | 0.001 | −0.549 | 0.46 | −0.228 | 0.767 | 0 | −0.086 | 0.209 | 0.167 |
| FAHD2A | 687850II [symbol] | 0.5245 | 0.0015 | −0.2015 | 0.2015 | −0.1995 | 0.0095 | 0.0975 | 0.5535 | 0.11 | −0.2285 | −0.3295 | 0.5635 | −0.3105 | 0.3325 | 0.5425 |
| FAM36A | 683002II [symbol] | 0.11 | 0.033 | 0.342 | 0.633 | −0.071 | −0.203 | −0.074 | 1.263 | 0.02 | −0.364 | −0.148 | 0.435 | 1.226 | −0.307 | −0.162 |
| FAM3A | 695471II [symbol] | −0.206 | 0.61 | 0.297 | −0.108 | 0.041 | −0.093 | 0.429 | −0.468 | −0.47 | −0.096 | −0.342 | 0.538 | 0.064 | 0.508 | −0.45 |
| FAM3B | 685659II [symbol] | 0.5095 | −0.2545 | 2.321 | −0.2825 | −0.3775 | −0.4015 | 0.4475 | −0.3775 | 0.76 | −0.0505 | 2.544 | −0.6825 | −0.0495 | −0.7155 | 2.92 |
| FAM3C | 681623II [symbol] | −0.149 | 0.242 | 0.025 | 0.052 | 0.493 | −1.271 | 0.191 | 0.921 | 0 | 0.289 | 0.468 | 0.362 | 0.991 | 0.822 | 0.177 |
| FAM46C | 685310II [symbol] | 1.756 | −0.1615 | −0.8775 | 0.9235 | 0.0155 | −0.5655 | 0.1635 | −0.8005 | −0.19 | −1.313 | −0.5375 | −0.4555 | −0.2645 | −0.0155 | 1.02 |
| FAM49B | 692836II [symbol] | −0.567 | 0.349 | −0.734 | −0.547 | −1.435 | −0.855 | 0.417 | 1.928 | −0.8 | 0.045 | −0.131 | 0.157 | −0.413 | 0.346 | 0.851 |
| FAM54A | 690904II [symbol] | 0.338 | 1.486 | 0.546 | 0.132 | 0.265 | 1.022 | 1.413 | 2.034 | −1.21 | 0.627 | −0.002 | 1.438 | 0.109 | −0.636 | 0.476 |
| FANCA | 685878II [symbol] | 0.195 | 0.467 | 0.475 | −1.005 | −0.05 | −0.736 | 1.575 | 2.675 | −1.03 | 0.085 | −1.046 | 2.321 | 1.481 | −0.577 | 2.355 |
| FANCE | 692746II [symbol] | −1.085 | −1.943 | 0.055 | −1.896 | −2.49 | −1.855 | −0.588 | −0.344 | 0.28 | 0.859 | −1.458 | −0.515 | −1.018 | −1.243 | −0.279 |
| FBL | 682815II [symbol] | −0.7515 | 0.1815 | 1.763 | 0.3025 | −0.4645 | 0.0225 | 1.067 | 0.4365 | 0.32 | 0.4905 | −0.6215 | −0.9805 | −0.3475 | −0.3535 | 0.6855 |
| FBP1 | 687231II [symbol] | −0.1147 | 0.1393 | 0.1433 | −0.7597 | −0.5677 | −0.4327 | 0.0783 | 0.7233 | 0.19 | −1.607 | 0.4553 | 1.081 | −0.7777 | −0.6017 | 0.9223 |
| FBS1 | 683642II [symbol] | −0.73 | 0.312 | −0.376 | −0.244 | 0.378 | 1.023 | −0.197 | −0.463 | 0.2 | −0.93 | 0.02 | 0.359 | 0.081 | −0.297 | 0.199 |
| FBXL20 | 696291II [symbol] | −0.263 | −0.209 | 0.177 | −0.157 | −0.547 | −0.282 | −0.021 | 0.283 | −0.35 | −0.101 | −0.305 | 0.106 | −0.248 | −0.072 | −0.08 |
| FBXO25 | 682373II [symbol] | −0.326 | −0.216 | 1.269 | 0.652 | 1.259 | 0.032 | 0.134 | 0.335 | 0.32 | −0.79 | −0.148 | −1.091 | −1.6 | −0.458 | −0.337 |
| FDXR | 696354II [symbol] | −0.990 | 0.121 | −1.422 | −0.023 | −0.171 | 0.169 | −1.476 | −0.896 | −0.43 | −1.193 | −0.205 | −0.823 | −0.673 | −1.578 | −0.521 |
| FEM1B | 687319II [symbol] | 0.117 | −0.473 | −0.453 | 0.029 | −0.264 | −0.443 | −0.179 | −0.378 | 0.19 | 1.242 | 0.071 | −0.643 | −0.297 | 0.027 | 0.3 |
| FGF12 | 686589II [symbol] | −0.2965 | 0.1855 | −0.7455 | −0.3325 | 1.351 | −0.0475 | −0.2875 | 0.0215 | −0.07 | −0.4985 | 0.2235 | −0.1965 | −0.1405 | −0.3625 | 0.1165 |
| FGF13 | 696768II [symbol] | 0.81 | 0.176 | 1.119 | −1.025 | −0.277 | −0.611 | 0.999 | 2.23 | 0.26 | 0.106 | 0.131 | 1.306 | 0.765 | −0.791 | 1.486 |
| FHOD1 | 693587II [symbol] | 0.172 | 0.198 | −1.138 | −0.38 | 0.016 | −0.248 | −0.029 | −0.933 | 0.06 | −0.018 | −0.595 | 0.09 | 0.994 | 0.03 | −0.96 |
| FKBP11 | 684453II [symbol] | 0.4105 | 0.2475 | −0.9825 | −0.2015 | −0.4115 | −0.2645 | −0.7475 | 0.4345 | −0.26 | 0.7895 | −0.4555 | 0.3295 | −0.8305 | −0.0445 | −0.3155 |
| FLJ10094 | 684307II [symbol] | −0.1165 | 0.0335 | −1.57 | 0.7535 | 3.232 | 0.2905 | −0.1545 | 0.1165 | 0.33 | −0.3645 | 0.3945 | −0.1925 | 0.7145 | −0.5245 | −0.0875 |
| FLJ10116 | 682368II [symbol] | 0.0985 | −0.1495 | −1.043 | 1.111 | 0.0355 | 0.5855 | −1.632 | 0.1715 | 0.73 | −1.655 | 0.5785 | −1.352 | 0.1585 | 0.4635 | −1.386 |
| FLJ10159 | 685392II [symbol] | −0.143 | 0.851 | −0.834 | −0.223 | 0.612 | −0.358 | −0.148 | −0.541 | 1.17 | −0.315 | 0.516 | −0.181 | −0.567 | −0.441 | −0.724 |
| FLJ10204 | 688075II [symbol] | −0.5865 | 0.3355 | 0.7535 | −0.0685 | −0.2925 | 0.7605 | 0.6325 | 2.023 | −0.49 | 0.2165 | −0.4855 | 0.1495 | 0.4435 | 0.0405 | 0.3005 |
| FLJ10276 | 694528II [symbol] | 1.501 | 1.061 | −0.512 | 1.11 | 1.811 | 1.332 | 0.835 | 0.255 | 0.42 | −0.549 | 1.083 | 1.29 | 0.736 | 0.211 | 0.701 |
| FLJ10292 | 686906II [symbol] | −0.035 | 0.343 | 1.665 | −0.004 | 1.352 | −0.025 | 1.267 | 2.237 | −0.67 | −0.64 | 1.138 | 1.758 | 0.004 | 0.811 | 2.196 |
| FLJ10324 | 693717II [symbol] | −1.338 | −0.776 | −2.288 | 0.843 | −0.505 | −0.783 | −1.553 | 0.228 | 0.57 | −0.329 | 0.292 | 2.347 | 0.566 | −1.997 | −3.051 |
| FLJ10700 | 687078II [symbol] | 0.1085 | −1.418 | 0.7295 | 0.0955 | −0.6315 | −0.9785 | −0.5155 | 0.7915 | 0.29 | −0.9145 | −0.3365 | 0.3945 | −0.5105 | −0.7125 | 1.113 |
| FLJ10706 | 693300II [symbol] | −1.408 | −1.272 | 0.403 | −1.257 | −2.074 | −2.052 | −0.03 | −0.16 | 0.08 | −0.481 | −1.876 | −0.507 | −0.445 | −1.191 | −0.063 |
| FLJ10871 | 694856II [symbol] | −0.202 | −0.42 | 0.767 | 0.407 | 0.041 | 1.296 | 0.009 | 0.06 | 0.02 | −0.296 | 0.303 | −0.318 | −0.579 | 0.002 | 0.288 |
| FLJ10901 | 684051II [symbol] | 1.03 | 1.035 | 1.349 | −1.563 | −0.5695 | −2.208 | 2.487 | 1.143 | −0.75 | 1.543 | −1.762 | 0.4455 | 1.967 | 1.437 | 1.672 |
| FLJ10916 | 695840II [symbol] | 0.481 | 0.012 | −0.41 | 0.409 | 0.3466 | −0.665 | −0.184 | 1.02 | 0.52 | −0.59 | 0.747 | 0.611 | 0.245 | −0.359 | 0.721 |
| FLJ10960 | 684797II [symbol] | 0.2535 | −1.079 | −2.704 | 0.7505 | −0.5265 | 1.461 | −1.44 | −1.975 | 0.89 | −1.812 | 0.7135 | −1.166 | −1.36 | 0.1816 | −0.9615 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| FLJ11017 | 682528II [symbol] | 0.629 | −0.106 | −0.566 | −0.341 | −0.283 | −0.368 | 0.075 | 2.59 | −0.09 | −1.012 | 0.412 | 0.255 | 1.369 | −0.272 | 1.621 |
| FLJ11088 | 689859II [symbol] | −0.024 | 0.365 | 1.948 | 1.463 | −0.011 | −0.123 | −0.752 | 0.097 | −0.02 | −0.634 | 0.522 | −0.32 | 0.291 | −0.384 | 1.683 |
| FLJ11151 | 695680II [symbol] | 0.017 | −1.059 | −0.128 | −0.604 | −1.528 | −0.149 | −0.047 | −0.463 | −0.29 | −1.361 | −0.684 | −2.252 | −0.333 | −0.093 | −1.317 |
| FLJ11287 | 688538II [symbol] | −0.2485 | 0.5275 | 0.0565 | 0.0005 | 0.0775 | 0.5555 | 0.1075 | −0.1725 | 0.24 | −0.7155 | 0.0365 | 0.8105 | −0.6575 | −0.2265 | 0.2215 |
| FLJ11280 | 697294II [symbol] | 0.053 | 1.202 | 0.536 | 1.856 | 1.722 | 1.529 | 0.443 | −0.045 | 0.38 | −0.778 | 1.475 | 0.629 | 1.643 | −0.275 | 0.501 |
| FLJ11506 | 691255II [symbol] | −0.1865 | 0.0625 | −0.7005 | −0.3355 | 0.2555 | 0.6045 | −0.1425 | −0.1965 | 0.17 | −0.0375 | 0.3065 | −0.2795 | −0.5615 | −0.3545 | −0.4855 |
| FLJ12270 | 690555II [symbol] | −0.4687 | 1.155 | −0.1747 | −0.6977 | 0.6603 | 0.0123 | 0.3463 | 0.3353 | 0.43 | −0.0398 | −0.2027 | 0.7573 | 0.3953 | −0.5267 | 0.2203 |
| FLJ12644 | 693818II [symbol] | −0.174 | −0.751 | 0.715 | −0.162 | 0.391 | 0.011 | −0.142 | −0.499 | 0.74 | −0.428 | 1.331 | −0.642 | 4.772 | 0.436 | 1.138 |
| FLJ12950 | 690008II [symbol] | −1.297 | 1.54 | −1.689 | 3.138 | −0.3295 | −1.604 | −1.739 | −1.776 | 0.88 | −1.451 | 1.964 | −1.687 | 1.098 | −2.064 | −1.585 |
| FLJ12684 | 685216II [symbol] | 0.247 | 0.066 | 0.167 | 0.104 | −0.254 | 0.952 | 0.243 | −0.298 | 0.04 | 3.319 | 1.06 | 0.325 | −0.317 | −0.08 | 1.715 |
| FLJ12735 | 697973II [symbol] | −0.4945 | 1.416 | 0.0325 | −0.4555 | −0.0325 | 0.8515 | 0.8965 | 1.338 | −0.4 | 0.3725 | −0.1425 | 0.8175 | 0.3445 | −0.5735 | −0.1515 |
| FLJ12750 | 685380II [symbol] | 1.834 | 0.8125 | −0.5385 | −0.2225 | 0.2285 | −0.3075 | 0.5165 | 0.3095 | −0.49 | 0.0865 | −0.8865 | 1.991 | −0.2085 | 0.6225 | 0.0145 |
| FLJ12895 | 683723II [symbol] | 0.1365 | −1.854 | 1.061 | 0.4725 | −1.531 | 0.1995 | −1.109 | −0.6195 | 0.32 | 0.4205 | 0.7355 | −0.9985 | −0.4235 | −1.771 | −0.1155 |
| FLJ13710 | 693274II [symbol] | −0.816 | 0.954 | −2.5 | 2.22 | 2.258 | 1.672 | −1.678 | −0.233 | 2.21 | −2.283 | 2.629 | −1.125 | 0.789 | −0.191 | −1.52 |
| FLJ13855 | 681758II [symbol] | −0.216 | 0.946 | −0.624 | −0.12 | −0.009 | 0.247 | 0.009 | 0.317 | 0.26 | −0.178 | 0.289 | −0.045 | −0.397 | 1.003 | 0.245 |
| FLJ13912 | 696739II [symbol] | −2.134 | −2.025 | −2.159 | −2.364 | −2.148 | −2.434 | −0.315 | −0.826 | 0.11 | 0.045 | −2.315 | −0.928 | −1.378 | −1.858 | −0.183 |
| FLJ14124 | 691127II [symbol] | −0.478 | 0.418 | 0.608 | 0.068 | 0.336 | −0.043 | 0.503 | 0.551 | 0.58 | −0.43 | 0.221 | 1.024 | −0.011 | 0.181 | 0.883 |
| FLJ14627 | 681398II [symbol] | −0.354 | −0.451 | 1.218 | 0.233 | 0.621 | −0.623 | 1.777 | 2.082 | −0.73 | −0.813 | −0.705 | 1.595 | 0.83 | −0.451 | 0.192 |
| FLJ14668 | 696627II [symbol] | −0.265 | −0.757 | −0.358 | −1.159 | −0.687 | −1.085 | 0.441 | −0.482 | 0.19 | −0.609 | −0.571 | −0.207 | 0.519 | −0.444 | 0.474 |
| FLJ14904 | 687755II [symbol] | −0.0983 | 0.3557 | 0.0238 | −0.1653 | 0.2737 | 0.3437 | −0.1853 | −0.1043 | 0.22 | −0.2333 | 0.4557 | 0.0128 | 0.5147 | −0.0853 | −0.2293 |
| FLJ20035 | 696171II [symbol] | −0.113 | 1.395 | −0.016 | 0.208 | 2.679 | 0.077 | 1.794 | 0.667 | −0.57 | −0.376 | 2.511 | 0.111 | 1.651 | 1.459 | 2.172 |
| FLJ20152 | 694420II [symbol] | −0.339 | 2.096 | −0.327 | 2.245 | 2.431 | 1.256 | −1.176 | −1.346 | −0.35 | −1.736 | 1.191 | 0.998 | 0.514 | −1.871 | −0.011 |
| FLJ20171 | 695239II [symbol] | −0.995 | 0.549 | −0.262 | −0.095 | −0.314 | 0.326 | −0.019 | −0.807 | −0.6 | −0.095 | 1.575 | −1.002 | −0.339 | −1.266 | −1.46 |
| FLJ20244 | 684905II [symbol] | 0.059 | −0.872 | 1.024 | −0.052 | 0.157 | 0.219 | 0.654 | −0.306 | 0.08 | 0.184 | −0.529 | −0.286 | −0.299 | 0.306 | 0.265 |
| FLJ20273 | 683092II [symbol] | −0.041 | 0.149 | −0.959 | 0.338 | −0.043 | 0.101 | −1.122 | −0.201 | 0.33 | 0.92 | −0.091 | −1.664 | 0.189 | 0.624 | −0.772 |
| FLJ20315 | 689446II [symbol] | −0.132 | 2.16 | −0.175 | −0.87 | 1.098 | −0.123 | −1.185 | 0.231 | 0.68 | −1.034 | 1.103 | −0.814 | −0.878 | 0.149 | −0.868 |
| FLJ20323 | 697352II [symbol] | −0.394 | 0.18 | 0.058 | 0.608 | 1.046 | −0.214 | 0.26 | −0.056 | 0.74 | 0.674 | 0.638 | −0.165 | −0.068 | −0.068 | −0.507 |
| FLJ20516 | 688292II [symbol] | −0.0435 | −1.147 | −0.2635 | 0.0195 | −0.4115 | −0.4125 | 0.4945 | 0.5155 | −0.58 | 2.139 | −0.7445 | −0.0085 | 0.1695 | −0.6355 | 0.5975 |
| FLJ20530 | 681463II [symbol] | −0.437 | 1.134 | 0.699 | −0.419 | 0.003 | 0.612 | 0.999 | 0.326 | 0.03 | 0.355 | 0.184 | 1.055 | −0.113 | 0.604 | 0.074 |
| FLJ20696 | 684792II [symbol] | −0.269 | −0.689 | 1.051 | −0.534 | −0.745 | 0.147 | 0.145 | −0.819 | 0.63 | −1.317 | 1.03 | −0.566 | 0.787 | −0.894 | 0.752 |
| FLJ20718 | 692255II [symbol] | 1.575 | −1.642 | −1.812 | 0.681 | 1.45 | −0.683 | −1.153 | 0.014 | −0.12 | 0.114 | 1.755 | −1.524 | −0.014 | 0.957 | 2.238 |
| FLJ20772 | 688898II [symbol] | −0.53 | 0.945 | 0.284 | −0.168 | −0.695 | 1.045 | 0.57 | 0.856 | −0.14 | 0.006 | −0.454 | 0.23 | −0.006 | 1.856 | 0.422 |
| FLJ20989 | 695210II [symbol] | −0.24 | 0.597 | 0.457 | −0.128 | −0.226 | 0.99 | 1.147 | 0.56 | −0.13 | 0.248 | −0.43 | 0.567 | −0.218 | −0.915 | −0.389 |
| FLJ21019 | 689334II [symbol] | −0.0335 | −0.0635 | −0.6245 | 0.4115 | 0.3215 | 0.1925 | −0.2805 | −0.2505 | 0.42 | −0.7335 | −0.1055 | 0.1685 | −0.3955 | −0.5455 | −0.7725 |
| FLJ21052 | 695900II [symbol] | −0.257 | 0.816 | 0.735 | 0.514 | 1.117 | 0.769 | −0.122 | 0.48 | 0.88 | −0.324 | 0.447 | 0.489 | −0.239 | 0.294 | 0.024 |
| FLJ21159 | 688476II [symbol] | 0.1055 | −0.9915 | 0.1175 | 0.9825 | 2.858 | 0.3595 | −1.566 | −0.6875 | 0.42 | −1.584 | 0.6265 | 0.3865 | −1.928 | −0.9035 | 0.5545 |
| FLJ21616 | 696280II [symbol] | −0.32 | −0.666 | 1.457 | 0.661 | −0.053 | 0.084 | 0.443 | 0.808 | 0.29 | −1.198 | −0.06 | 0.002 | −0.115 | −1.46 | 0.891 |
| FLJ21827 | 683572II [symbol] | 0.0115 | −0.4035 | 0.5675 | 0.9115 | 0.3615 | 0.7135 | −0.9785 | −0.7935 | 0.89 | −1.111 | −0.3335 | −1.179 | −1.825 | 0.4445 | −0.7915 |
| FLJ21963 | 688106II [symbol] | 0.724 | 0.419 | 0.304 | −0.466 | −0.493 | −0.747 | −0.126 | 0.784 | 0.95 | 0.374 | −0.409 | −0.201 | 0.23 | −0.337 | 0.784 |
| FLJ22104 | 690673II [symbol] | −0.203 | 1.031 | 0.23 | −0.38 | 2.028 | 1.058 | −1.016 | 0.358 | 0.11 | 0.225 | −0.38 | −0.04 | −0.353 | 0.514 | −0.406 |
| FLJ22222 | 694558II [symbol] | 0.61 | 1.003 | −0.284 | 0.632 | 0.205 | −0.28 | −0.451 | 1.078 | −0.14 | −0.004 | 0.349 | 1.113 | −0.031 | −1.16 | 0.042 |
| FLJ22573 | 683583II [symbol] | −1.195 | −0.2558 | −0.1528 | −0.6398 | −0.2248 | −0.4858 | 0.6792 | −0.0408 | −0.68 | −0.4038 | 0.1452 | −0.2728 | −0.4308 | −0.5238 | 1.117 |
| FLJ22794 | 692448II [symbol] | 0.543 | −0.723 | −0.045 | −0.066 | 0.107 | 0.441 | −0.251 | 0.412 | 0.49 | −2.253 | 0.278 | −0.749 | −0.659 | 0.933 | 0.321 |
| FLJ23186 | 692984II [symbol] | −0.04 | 2.681 | 0.572 | 0.394 | 0 | −0.26 | 0.074 | 0.485 | 0.35 | 0.703 | 1.236 | 0.002 | −0.76 | −0.286 | −0.212 |
| FLJ23441 | 696416II [symbol] | 0.262 | −0.509 | −0.123 | 0.736 | 0.582 | 0.485 | 0.058 | −0.079 | −0.3 | −0.007 | 1.125 | 0.406 | 0.343 | 0.677 | 0.255 |
| FLJ25471 | 695557II [symbol] | −0.147 | −0.405 | 0.432 | −0.303 | −0.053 | −0.112 | 0.083 | −0.55 | −0.13 | 0.895 | −0.667 | −0.234 | −0.346 | −0.22 | 0.09 |
| FLJ31204 | 683963II [symbol] | −0.483 | −0.155 | 1.227 | 0.341 | 0.814 | 1.132 | −0.875 | −0.072 | 0.67 | −1.797 | 0.416 | −2.614 | 0.27 | −0.639 | 0.689 |
| FLJ31795 | 688302II [symbol] | −0.4205 | −0.3635 | −0.1185 | −0.0255 | −0.0085 | −0.2985 | 0.0365 | 0.6445 | 0.11 | 1.38 | −0.5355 | 0.2275 | 0.2945 | −0.7275 | 0.0745 |
| FLJ32942 | 690691II [symbol] | 0.086 | 0.226 | −0.139 | −0.085 | 0.256 | 0.347 | 0.001 | −0.267 | −0.25 | 0.84 | −0.166 | 0.302 | −0.001 | 0.028 | −0.229 |
| FLJ37970 | 697729II [symbol] | 0.593 | −1.559 | 0.215 | −0.863 | −0.6838 | −1.59 | −0.124 | 0.003 | −0.31 | −1.141 | −1.035 | 0.569 | −0.474 | −0.366 | 0.584 |
| FLJ39370 | 694709II [symbol] | 1.836 | 1.025 | −2.038 | 2.274 | 0.414 | 1.029 | −0.805 | −0.406 | 0.48 | −1.763 | 1.212 | −1.475 | 0.752 | −1.019 | 0.475 |
| FLJ90586 | 685670II [symbol] | −2.044 | 0.951 | 1.5 | 1.566 | 0.488 | −2.402 | 2.4 | 1.401 | 0.68 | −1.341 | −1.85 | 1.87 | 0.819 | −1.565 | 0.388 |
| FN3KRP | 695326II [symbol] | 0.174 | 1.16 | −0.186 | 0.095 | −0.091 | 0.079 | −0.388 | −0.058 | −0.17 | 0.128 | −0.083 | 0.143 | −0.16 | −1.133 | −0.38 |
| FOLR1 | 697855II [symbol] | −1.738 | −1.88 | −1.687 | −2 | −1.811 | −1.323 | −1.956 | −0.705 | 0.14 | −0.513 | −1.657 | 0.826 | −1.068 | −0.3 | −1.199 |
| FOXA1 | 692889II [symbol] | 1.99 | 2.045 | −3.9 | 3.12 | 2.665 | 1.527 | −4.24 | −3 | 0.83 | −4.04 | 1.583 | −4.398 | 1.428 | 2.116 | −3.51 |
| FOXC1 | 772435II [symbol] | 0.408 | −1.27 | 3.614 | −1.075 | −1.37 | −0.71 | 2.223 | 1.438 | 0.35 | −1.125 | −1.27 | 2.87 | −0.55 | −1.33 | 2.368 |
| FOXP1 | 692043II [symbol] | −0.017 | 0.294 | −1.296 | −0.025 | −0.726 | −0.352 | −1.076 | −0.805 | 0.33 | −1.826 | 0.049 | −1.929 | −0.928 | 0.221 | −1.513 |
| FREQ | 683314II [symbol] | −0.4622 | 0.2368 | 0.7408 | −0.6532 | 0.2048 | −0.4812 | 1.803 | 1.596 | −0.71 | 1.527 | −0.8412 | 0.4978 | 0.5028 | 0.3248 | 1.261 |
| FTO | 693312II [symbol] | 0.369 | 0.427 | −0.478 | 1.282 | 2.741 | −0.442 | −0.119 | −0.237 | 1.22 | −0.16 | 1.524 | 1.207 | 1.545 | 0.139 | 0.04 |
| FUBP1 | 690423II [symbol] | −0.567 | −0.532 | −1.089 | −0.434 | −0.776 | −0.758 | 0.175 | 0.868 | −0.32 | 0.679 | −0.579 | 0.531 | 0.094 | −0.216 | 0.602 |
| FUCA1 | 691254II [symbol] | 0.5155 | −0.1175 | −0.9075 | 1.093 | 1.549 | −0.0985 | −0.0825 | −0.5695 | −0.14 | −0.4265 | 0.5995 | −0.6595 | −0.5595 | 0.1705 | −0.9275 |
| FUT8 | 695197II [symbol] | 0.436 | 0.479 | −2.239 | −0.748 | −0.923 | 0.929 | −1.134 | −1.054 | 0.95 | 0.654 | 1.534 | −1.317 | 0.376 | −0.311 | 0.868 |
| FXYD8 | 687048II [symbol] | 0.0475 | 0.0575 | 0.3965 | −0.0755 | −0.8995 | −0.2335 | 0.2815 | −0.9155 | 0.82 | −0.9055 | 0.0225 | 0.4045 | −0.6785 | −1.004 | −0.1305 |
| FYCO1 | 691750II [symbol] | 0.022 | 0.088 | −1.033 | 0.427 | 0.393 | −0.632 | −0.871 | −1.076 | 0.86 | −1.182 | −0.022 | −1.54 | −0.614 | 0.285 | 0.807 |
| FZD10 | 686248II [symbol] | −0.023 | −0.902 | 4.678 | 0.931 | 0.105 | 0.198 | −0.65 | −1.134 | 0.46 | 2.984 | −0.475 | −0.758 | 0.074 | 0.012 | −1.139 |
| FZD7 | 691443II [symbol] | −0.945 | −0.475 | 1.447 | 0.577 | −2.15 | −0.742 | −0.968 | −0.038 | 1.64 | −1.307 | 0.066 | 0.33 | −1.281 | −0.495 | 1.544 |
| FZD9 | 691926II [symbol] | −0.497 | −0.926 | −0.539 | −0.319 | −0.521 | −0.464 | −0.508 | −0.798 | −0.5 | −0.058 | −0.743 | −0.823 | −0.635 | 0.369 | −0.226 |
| G1P2 | 694822II [symbol] | 0.224 | 2.011 | 0.125 | −0.485 | 3.91 | 0.44 | 2.816 | 1.291 | −1.65 | 1.019 | 2.991 | 1.465 | 2.535 | 2.583 | 2.075 |
| G1P3 | 693731II [symbol] | 0.289 | 0.826 | −0.168 | 0.334 | 2.084 | 0.213 | 2.916 | 0.507 | −1.39 | 1.381 | 3.352 | 1.255 | 1.79 | 2.804 | 1.623 |
| GA17 | 696445II [symbol] | −0.885 | −1.714 | 0.303 | −0.938 | −1.167 | −1.533 | −0.959 | −0.181 | 0.03 | −0.089 | −1.58 | −2.232 | −1.193 | −0.86 | −0.386 |
| GABARAF | 683657II [symbol] | −0.9585 | 0.6265 | −1.193 | 0.0105 | 0.4095 | −0.4725 | −0.0455 | 0.3025 | 0.35 | 0.7465 | −0.0105 | −0.1335 | 0.2155 | −0.4245 | 0.9115 |
| GAJ | 696722II [symbol] | −1.832 | −3.297 | −0.558 | −3.036 | −3.141 | −3.297 | −0.775 | −1.332 | −1.18 | −1.523 | −3.252 | −0.792 | −1.82 | −3.374 | −1.245 |
| GALE | 681740II [symbol] | 0.587 | 0.736 | −0.541 | −0.173 | 0.733 | 0.365 | 0.147 | −0.918 | −0.44 | −0.016 | −0.749 | 0.05 | 0.308 | 0.89 | −0.747 |
| GALNT14 | 693376II [symbol] | −2.035 | −1.332 | 1.114 | −0.0535 | 1.395 | −0.4085 | −0.7805 | 0.0535 | 0.61 | −0.7395 | −1.009 | 0.4345 | −1.637 | −2.124 | 0.2865 |
| GALNT2 | 692425II [symbol] | −0.14 | −1.417 | 0.412 | −0.465 | −1.086 | −1.368 | −0.164 | 0.253 | −0.29 | 0.783 | −1.287 | 1.139 | 0.008 | 0.45 | −0.031 |
| GALNT7 | 684872II [symbol] | 2.037 | −0.131 | −1.377 | 1.354 | 1.544 | −0.173 | −1.21 | −0.364 | 0.73 | −1.521 | 0.181 | −1.173 | −0.219 | 0.033 | −0.62 |
| GAMT | 697858II [symbol] | −0.03 | 0.026 | −2.028 | 0.567 | 0.228 | −0.142 | −1.211 | −1.641 | 0.77 | −0.53 | −0.31 | −0.078 | 0.054 | −0.711 | −2.374 |
| GAPD | 696932II [symbol] | −0.972 | −1.664 | −0.233 | −1.977 | −1.33 | −1.903 | −2.35 | −0.678 | −0.31 | −0.391 | −1.834 | −1.563 | −0.579 | −0.479 | −0.918 |
| GARS | 693904II [symbol] | −0.075 | −0.419 | −0.917 | −0.92 | 0.014 | −0.949 | −0.158 | −0.654 | −0.53 | 0.178 | −1.343 | −0.824 | −0.915 | −0.612 | −0.448 |
| GART | 683079II [symbol] | −0.007 | 0.189 | 0.814 | −0.055 | 0.576 | −0.247 | 0.622 | 1.385 | 0.01 | 1.439 | −0.265 | 1.123 | 0.231 | 0.08 | 0.918 |
| GATA3 | 696046II [symbol] | 0.198 | 1.397 | −2.394 | 2.468 | 2.365 | 2.21 | −2.824 | −1.131 | 0.92 | 0.801 | 1.489 | −1.164 | 0.93 | 0.441 | −1.536 |
| GATM | 685950II [symbol] | −0.87 | −0.042 | 1.301 | 0.477 | 0.177 | 1.76 | −1.198 | −0.807 | 0.14 | −2.253 | 0.717 | −0.717 | −1.268 | −0.265 | 1.477 |
| GBA | 691089II [symbol] | 0.1235 | −0.5515 | −0.6775 | 0.0435 | 0.1225 | 0.4755 | 0.7795 | −0.8865 | −0.28 | −0.5115 | −0.3665 | 1.949 | −0.1305 | 0.3075 | 0.1165 |
| GDAP1 | 689715II [symbol] | −1.232 | −0.3415 | 0.1055 | 1.988 | 1.511 | 1.797 | −0.9245 | −0.2495 | 0.83 | −1.237 | 0.3965 | −0.2135 | 0.0165 | −1.664 | −0.1355 |
| GDI2 | 686228II [symbol] | 0.1015 | −0.0545 | 1.276 | −0.1945 | −0.3585 | −0.5105 | 0.0515 | 0.9835 | −0.27 | 0.7185 | −0.0765 | −0.5345 | 0.3165 | 0.5905 | 1.702 |
| GGA1 | 683921II [symbol] | 0.0545 | −0.0645 | −0.8015 | −0.0515 | 0.5015 | 0.5905 | 0.1955 | 0.4175 | 0.3 | −0.4385 | 0.0515 | −0.1015 | 0.2405 | 0.0515 | 0.0595 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| GKAP1 | 682770II [symbol] | −0.3225 | 0.1395 | 0.3515 | 0.7575 | 0.8105 | 0.3345 | 0.1985 | 0.4115 | 0.52 | −2.076 | −0.0095 | −1.217 | −1.007 | −0.6165 | 0.0375 |
| GLCCI1 | 689756II [symbol] | 0.2525 | 0.4785 | −1.966 | −0.2325 | 0.7475 | 0.4455 | −0.8625 | −1.296 | 0.15 | −0.6565 | −1.05 | −1.13 | −0.4985 | −0.7565 | −0.0795 |
| GLG1 | 681938II [symbol] | −0.0485 | 0.5635 | −0.2985 | −0.4625 | 0.8285 | −0.4815 | −0.0985 | 0.0545 | 0.02 | −0.1445 | 0.0515 | 0.7985 | −0.0335 | −0.3385 | −0.5795 |
| GLTSCR2 | 686786II [symbol] | −0.619 | −0.359 | 0.646 | 0.866 | −0.14 | −0.043 | −0.252 | −0.64 | 0.51 | −0.453 | 0.043 | −1.124 | −1.476 | −0.044 | −0.529 |
| GNB4 | 697071II [symbol] | −0.984 | −0.268 | −1.441 | −0.843 | −0.33 | −0.532 | 0.229 | 0.927 | 0.05 | 0.114 | −0.227 | −0.794 | −0.15 | 0.273 | 1.716 |
| GOLPH2 | 689832II [symbol] | −0.1725 | −0.0045 | −1.231 | 0.0045 | 0.2915 | 0.6045 | −0.6475 | −0.1635 | 0.65 | −1.165 | 0.4215 | 0.3505 | −0.7785 | 0.6195 | −0.6335 |
| GPATC1 | 687697II [symbol] | −0.576 | −1.081 | −0.375 | −0.078 | −0.013 | 0.351 | 0.494 | 0.141 | 0.29 | 0.334 | −0.498 | −1.154 | −0.486 | −1.01 | 0.069 |
| GPC2 | 692352II [symbol] | 0.358 | −0.947 | −1.344 | 0.992 | −0.505 | 0.061 | −1.817 | −0.122 | −0.06 | −1.59 | 0.32 | −1.147 | 0.861 | 0.05 | −0.132 |
| GPR125 | 694724II [symbol] | −1.345 | −0.108 | 1.29 | −1.726 | −0.147 | −0.541 | 0.243 | 0.144 | 0.71 | −1.357 | −0.91 | 1.732 | −0.118 | −0.797 | 0.019 |
| GPR160 | 682327II [symbol] | 0.159 | 1.06 | −2.876 | 1.142 | −1.203 | 0.973 | −0.623 | −0.624 | 0.52 | −1.704 | 1.892 | −1.694 | −1.176 | 1.861 | −0.013 |
| GPR56 | 688865II [symbol] | −0.7025 | 1.189 | −0.0375 | −0.6945 | 0.5995 | −0.7385 | 0.9015 | 0.2195 | −0.09 | −1.186 | −1.487 | 1.106 | 0.2065 | 0.5605 | 0.4855 |
| GPRC5C | 696709II [symbol] | 1.094 | 0.013 | −1.262 | 0.735 | −0.113 | −0.995 | −1.639 | −0.737 | 0.6 | −0.382 | −0.024 | −0.838 | 0.634 | −0.003 | −2.059 |
| GPSM1 | 682326II [symbol] | 0.1197 | 0.0848 | 0.1847 | −0.8553 | −0.2733 | −0.8993 | 0.3717 | −0.7213 | 0.35 | 0.0428 | −0.1383 | 0.6677 | 0.4967 | −0.4043 | −0.7643 |
| GPSM2 | 690902II [symbol] | 0.929 | 0.781 | −1.627 | 0.492 | −0.25 | 0.095 | −1.598 | −1.412 | −0.6 | −2.325 | −0.147 | 0.005 | −0.078 | 0.472 | −1.895 |
| GPT | 693608II [symbol] | −0.377 | −0.747 | −1.468 | −0.815 | 0.957 | 0.303 | −1.38 | 0.374 | 0.21 | −0.817 | 0.613 | 0.235 | −0.554 | −0.075 | −0.832 |
| GPT2 | 689304II [symbol] | 1.108 | 1.081 | 0.442 | −1.182 | 2.301 | 1.347 | 2.299 | 1.135 | −0.1 | −0.697 | −1.217 | 1.52 | 0.032 | 1.081 | 1.178 |
| GPX7 | 694626II [symbol] | 0.344 | 0.347 | 2.301 | −1.111 | 0.019 | −0.819 | 1.405 | −0.335 | 0.36 | −0.094 | 0.598 | 0.212 | −1.053 | −1.217 | 0.741 |
| GRB14 | 691136II [symbol] | 0.9502 | 0.2632 | 2.983 | −0.5178 | −0.2148 | −0.1468 | 3.955 | 3.993 | −0.88 | −0.8918 | 1.7 | 1.623 | 4.492 | −0.3378 | 2.705 |
| GRB7 | 691667II [symbol] | 1.126 | 0.076 | −0.26 | −0.006 | −0.498 | 0.409 | 0.005 | −0.005 | −0.05 | −0.279 | 0.066 | −0.011 | −0.128 | 3.045 | −0.348 |
| GSC | 684798II [symbol] | 1.392 | 0.2133 | −0.8827 | −0.4597 | 0.4573 | 0.0713 | −0.8977 | −1.535 | 0.64 | −0.6867 | 0.4823 | 0.3013 | 0.2943 | −0.6447 | −1.111 |
| GSDML | 684490II [symbol] | 1.277 | −1.403 | 0.55 | −1.64 | −2.831 | −0.685 | −2.501 | 0.136 | 0.05 | −1.87 | −0.105 | −1.594 | 0.106 | −0.109 | 0.196 |
| GSK3B | 682653II [symbol] | −0.336 | 1.289 | −0.938 | −0.109 | 0.04 | 0.21 | −0.614 | 0.053 | −0.46 | −1.014 | −0.048 | −0.51 | 2.331 | −1.025 | −0.4594 |
| GSTA1 | 690111II [symbol] | −0.002 | 0.249 | 0.376 | −0.164 | 0.44 | 0.193 | 0.065 | 0.377 | 0.13 | 0.038 | 0.035 | 1.175 | −0.117 | −0.066 | 0.233 |
| GSTK1 | 691113II [symbol] | −0.558 | −0.129 | −1.17 | −0.317 | −0.274 | 0.802 | 0.161 | −0.655 | 0.11 | −1.747 | −1.049 | 0.163 | −0.827 | −0.034 | −0.34 |
| GSTM3 | 686813II [symbol] | −0.631 | 0.903 | −0.432 | 2.272 | −1.119 | 3.235 | −0.428 | −1.004 | 0.55 | −1.212 | 2.351 | −0.71 | 0.886 | −0.83 | −1.242 |
| GSTP1 | 690952II [symbol] | −0.527 | −1.246 | 1.279 | −1.327 | −0.204 | 1.361 | 1.285 | 0.707 | 0.24 | 1.624 | −0.71 | 1.598 | −1.642 | −1.896 | 1.36 |
| GSTT1 | 684076II [symbol] | −1.89 | −0.536 | −0.71 | −1.75 | −1.948 | −1.119 | −0.76 | 0.52 | 0.28 | −1.25 | −1.75 | −1.302 | −0.511 | 1.03 | 0.2 |
| GSTT2 | 685053II [symbol] | −0.35 | −0.094 | −0.613 | −0.695 | −0.297 | 0.695 | −0.859 | −0.983 | 0.838 | −0.623 | 0.19 | −0.223 | 0.063 | −0.187 | 1.067 |
| GTF2F2 | 697621II [symbol] | −0.74 | −0.247 | 0.094 | −1.128 | 0.19 | −1.547 | −0.43 | −0.145 | 0.05 | −0.354 | −1.387 | −1.063 | −0.448 | −0.856 | −0.24 |
| GTF2I | 692303II [symbol] | −0.211 | −0.273 | 0.636 | 0.807 | 0.697 | 1.17 | −0.126 | 0.16 | 0.9 | −1.385 | 0.111 | 0.415 | 0.044 | −0.516 | −0.522 |
| GTF3A | 693886II [symbol] | 0.345 | −1.134 | 0.443 | −0.335 | −0.841 | −0.661 | 0.908 | 1.061 | 0.07 | −1.028 | −0.528 | 0.563 | 0.072 | −0.033 | 2.329 |
| GTPBP3 | 682310II [symbol] | 0.7385 | 0.0735 | 1.312 | −2.408 | 0.5265 | −1.309 | 0.4775 | 0.7675 | −0.36 | 1.632 | −0.7785 | 0.2575 | −0.1005 | 0.1235 | −0.0005 |
| GTPBP4 | 689649II [symbol] | 0.0165 | 0.4915 | 1.18 | −0.6815 | 0.1015 | −0.0175 | 0.8005 | 1.722 | −0.36 | 0.8525 | −0.1195 | 0.6045 | 0.6805 | 0.6675 | 2.41 |
| H1F0 | 692476II [symbol] | −0.4245 | 0.3335 | −0.1905 | −0.2925 | 0.1185 | 0.0575 | 0.6555 | 0.6605 | 0.81 | −0.2515 | 0.6685 | 0.6515 | −0.8065 | 0.4305 | 1.657 |
| H2AFV | 695317II [symbol] | −0.622 | −0.829 | −1.181 | −0.555 | −1.039 | −0.496 | −0.15 | −0.437 | −0.47 | 0.669 | −0.431 | −0.597 | −0.076 | −1.278 | −0.132 |
| H2AFY | 696406II [symbol] | 0.946 | 0.835 | 0.11 | 0.599 | 0.921 | 0.98 | 0.634 | −1.228 | −0.46 | 0.601 | 0.65 | 0.101 | 1.137 | 0.658 | 0.388 |
| H2AFZ | 683032II [symbol] | 0.3335 | 0.2695 | 0.1765 | 0.0355 | −0.8385 | −0.2355 | 1.411 | 0.8905 | −0.71 | 1.386 | −0.0035 | 0.1175 | 0.6915 | −0.8595 | 1.737 |
| H2BFS | 685337II [symbol] | 0.3425 | −0.0105 | 0.5185 | −0.2115 | 0.5795 | −1.192 | −0.1105 | 1.157 | −0.26 | 0.0695 | −0.8105 | −0.1435 | 0.2965 | 0.0965 | −0.1065 |
| H3F3B | 690295II [symbol] | 0.207 | 0.991 | −0.528 | −0.082 | −0.554 | 0.154 | −0.726 | 0.928 | 0.13 | −0.154 | −0.004 | −0.344 | 0.023 | −1.338 | −0.843 |
| H41 | 682811II [symbol] | 0.7835 | −0.2205 | 1.611 | −0.3855 | −0.2905 | 0.5635 | 2.192 | 1.694 | −0.03 | 1.754 | −0.2475 | 1.736 | 0.7255 | 0.3905 | 1.399 |
| HAGH | 696380II [symbol] | 0.206 | 0.628 | −1.043 | 0.839 | 1.268 | 1.645 | 0.386 | −0.442 | 0.01 | −0.876 | 0.037 | −0.095 | 0.092 | −0.37 | −0.217 |
| HBG1 | 688388II [symbol] | 0.133 | 0.052 | 0.188 | 0.149 | 0.038 | 0.088 | 0.078 | −0.616 | 0.21 | −0.869 | 0.18 | −0.503 | −0.542 | 0.223 | 0.4 |
| HBLD1 | 697677II [symbol] | −0.306 | −0.049 | −0.219 | −0.598 | −0.437 | −0.025 | −0.861 | −0.76 | 0.39 | 0.634 | 0.085 | −1.201 | −0.291 | −0.064 | −0.39 |
| HCAP-G | 691867II [symbol] | 0.758 | 0.787 | 0.759 | −0.618 | −0.137 | −0.548 | 2.302 | 1.426 | −0.69 | 0.93 | −0.222 | 1.196 | 0.556 | 0.868 | 0.632 |
| HCP5 | 695382II [symbol] | 1.064 | 0.838 | 0.077 | 0.587 | 3.674 | 1.64 | 1.634 | 0.833 | −0.64 | 0.187 | 1.812 | 1.288 | 1.762 | 1.222 | 3.443 |
| HDAC2 | 697848II [symbol] | −0.238 | −0.597 | 1.107 | −0.808 | −0.395 | −1.094 | −0.049 | 0.228 | 0.13 | 0.062 | −0.568 | 0.355 | 0.074 | −0.468 | 0.194 |
| HDC | 688061II [symbol] | −0.0475 | −0.8045 | −0.2205 | −0.2135 | 4.522 | −0.5255 | 0.6185 | −0.3775 | 1.04 | −0.1785 | −0.0385 | −0.5425 | −0.2215 | 0.0995 | −0.3745 |
| HEBP1 | 697035II [symbol] | −0.2 | 1.645 | 2.496 | 1.092 | 1.766 | 1.164 | −0.573 | −0.68 | 0.56 | −1.261 | 0.853 | −0.375 | 0.035 | −0.657 | 0.935 |
| HIPK2 | 691802II [symbol] | 0.4235 | 0.134 | −0.0675 | −0.2545 | 0.2752 | 0.1665 | −0.0485 | −0.2595 | 0.03 | 0.0175 | 0.3995 | 0.0155 | −0.2445 | −0.2635 | 0.0835 |
| HIPK2 | 694742II [symbol] | 0.863 | 0.139 | 1.091 | 2.233 | 2.653 | −0.372 | 1.343 | 1.506 | 0.42 | −1.338 | 0.574 | 1.688 | 1.397 | 0.579 | 0.163 |
| HIS1 | 694010II [symbol] | −0.132 | −0.114 | −1.004 | 0.261 | 0.312 | 0.762 | −0.209 | 0.519 | 0.82 | 0.605 | 0.636 | −0.003 | −0.264 | 0.247 | −0.114 |
| HIST1H2A | 684611II [symbol] | −0.6587 | 0.7373 | 0.3763 | −0.7707 | 0.6803 | −0.8087 | −0.8547 | 1.085 | −0.31 | −1.492 | −0.8697 | 0.1093 | 0.0133 | −1.014 | −1.615 |
| HIST1H2B | 697885II [symbol] | −1.864 | 2.045 | −1.721 | 3.511 | 4.254 | 2.596 | −2.762 | −1.523 | −0.22 | −2.101 | 2.488 | −3.744 | 0.992 | −2.325 | −1.251 |
| HIST1H2B | 681566II [symbol] | 0.45 | 0.003 | 0.36 | −0.073 | 0.596 | −1 | −0.619 | 1.118 | −0.34 | −0.457 | −0.712 | 0.048 | −0.003 | −0.167 | −0.789 |
| HIST1H2B | 695646II [symbol] | 0.253 | −0.058 | 0.24 | −0.197 | 0.414 | −0.892 | −0.694 | 0.995 | −0.26 | −0.541 | −0.541 | −0.06 | −0.077 | 0.236 | −0.729 |
| HIST1H2B | 683833II [symbol] | 0.2295 | 0.0505 | 0.0675 | −0.3165 | 0.7535 | −0.7645 | −0.9515 | 1.053 | −0.35 | −0.6275 | −0.4935 | −0.0145 | −0.3165 | −0.4735 | −0.9165 |
| HIST1H3C | 693912II [symbol] | −0.084 | 0.973 | −0.782 | −0.471 | −0.397 | −0.403 | −1.142 | 0.64 | −0.32 | −0.492 | −0.367 | −0.224 | −0.382 | −1.439 | −1.147 |
| HM13 | 695097II [symbol] | 0.538 | −0.707 | −0.189 | −0.122 | −0.222 | 0.003 | 0.275 | 0.023 | −0.1 | −0.213 | −0.134 | −0.411 | 0.776 | 0.255 | −0.038 |
| HMGB3 | 690848II [symbol] | 0.974 | 1.583 | 0.788 | 1.315 | −1.151 | −1.343 | 2.657 | −0.59 | −0.62 | −2.801 | 2.015 | 1.222 | −0.621 | 0.061 | 1.381 |
| HN1 | 689578II [symbol] | 0.836 | 0.998 | 0.541 | −0.355 | 0.099 | −0.07 | 0.044 | 1.052 | −1.01 | 0.398 | −0.114 | 1.387 | 1.725 | −0.044 | 0.3 |
| HNRPA3P | 687868II [symbol] | −0.0285 | −0.4055 | 1.016 | −0.1975 | −0.5425 | 0.0675 | 0.3945 | 0.8035 | −0.28 | 0.9315 | −0.3185 | −0.1665 | 0.2815 | −0.0595 | 0.7825 |
| HNRPDL | 689713II [symbol] | −0.2353 | 0.0778 | 0.0408 | 0.1947 | −0.0663 | −0.3933 | 0.0686 | 0.4537 | −0.32 | 1.155 | −0.3823 | −0.3623 | 0.0738 | 0.1817 | 0.4757 |
| HOXA5 | 682213II [symbol] | −0.033 | −0.149 | −0.584 | 2.55 | 0.192 | −0.034 | −0.588 | −1.06 | 1.68 | 1.637 | 0.48 | 2.699 | −0.656 | −0.0595 | −0.486 |
| HOXA7 | 693224II [symbol] | 0.155 | 0.955 | −1.089 | 3.703 | −0.169 | −0.25 | −0.641 | −2.558 | 2.77 | 3.112 | 0.442 | 3.383 | −0.905 | −0.682 | −0.129 |
| HOXB2 | 695779II [symbol] | 0.375 | −0.225 | −2.749 | 2.542 | 2.335 | −1.879 | −1.619 | −2.514 | 0.08 | −1.223 | 0.739 | −1.444 | −1.234 | 0.348 | −1.615 |
| HOXB6 | 688928II [symbol] | −0.573 | −0.979 | −1.782 | −1.039 | −2.034 | −1.604 | −1.289 | −1.527 | −0.1 | −0.951 | −1.729 | −2.077 | −0.943 | −1.077 | −1.466 |
| HOXB7 | 687026II [symbol] | 0.481 | 0.445 | −2.218 | 1.575 | 1.741 | −0.824 | −1.089 | −1.252 | −0.55 | 0.508 | −0.704 | −0.501 | −1.559 | 0.016 | −1.106 |
| HOXC10 | 688125II [symbol] | 0.6015 | −0.2635 | −1.429 | −0.5215 | −1.211 | 0.6665 | 0.1165 | −2.322 | 0.44 | −1.418 | −1.246 | −0.2615 | −0.0175 | 0.2315 | −2.381 |
| HOXC13 | 688022II [symbol] | −0.1605 | 0.1865 | −0.2105 | −0.0375 | 0.4545 | 0.2215 | −0.2995 | −0.0595 | 0.06 | 0.0815 | −0.0225 | −0.1525 | −0.3055 | 0.0395 | −0.3975 |
| HOXC9 | 686974II [symbol] | −0.13 | −1.492 | −0.46 | −0.882 | −0.906 | −0.546 | 0.294 | −0.204 | 0 | −0.61 | −1.224 | −0.679 | 0.297 | −0.259 | −0.969 |
| HOXD3 | 688881II [symbol] | −0.3365 | −1.305 | −1.752 | −0.4085 | 0.7225 | 0.1875 | −1.485 | 0.3035 | 0.61 | −0.3205 | 0.7585 | −0.0115 | −0.2965 | 0.5135 | −0.2725 |
| HPCL2 | 693166II [symbol] | −1.08 | −0.875 | 0.135 | −0.816 | −0.309 | −0.747 | −1.165 | −1.045 | 0.25 | 0.14 | −0.081 | −1.169 | −1.595 | −1.262 | 0.042 |
| HPS3 | 684956II [symbol] | −0.093 | 0.512 | −0.473 | 0.187 | 0.003 | −0.024 | −0.473 | 0.207 | −0.04 | −0.593 | 0.267 | −0.489 | −0.642 | 0.303 | 1.097 |
| HRASLS | 689305II [symbol] | 0.814 | −1.361 | 0.78 | −0.093 | −0.77 | −1.124 | 0.5651 | 3.324 | −0.96 | −1.452 | −1.279 | 0.664 | 0.017 | 1.096 | 2.491 |
| HRB | 692071II [symbol] | 0.2815 | 0.4785 | 0.7995 | −0.7525 | −0.0355 | −0.4175 | −0.1215 | 1.774 | −0.37 | 1.174 | 0.2105 | 0.1065 | −0.2305 | 0.6195 | 1.279 |
| HRSP12 | 681060II [symbol] | −1.286 | 1.674 | 0.901 | −0.625 | 0.003 | −0.096 | 0.827 | 0.702 | −0.28 | 0.754 | 0.452 | 0.629 | −0.067 | 0.915 | 0.931 |
| HSD17B2 | 692999II [symbol] | 1.247 | −1.716 | 0.368 | −0.925 | −1.308 | −1.169 | −0.859 | 1.35 | 0.32 | −1.316 | −1.684 | −0.973 | −1.157 | 0.198 | 0.078 |
| HSF1 | 694939II [symbol] | −1.192 | 1.278 | −1.315 | −0.001 | −0.166 | −0.879 | −0.532 | −1.337 | −0.37 | −1.03 | 2.03 | −1.354 | −0.489 | 0.042 | −2.123 |
| HSPA14 | 694810II [symbol] | 0.211 | 0.585 | −1.188 | 0.392 | 0.218 | 0.154 | −0.883 | −1.109 | −0.39 | −0.275 | 0.302 | 0.246 | −0.01 | −0.157 | −0.698 |
| HSPA2 | 695880II [symbol] | 1.112 | 1.749 | −0.066 | 1.54 | 0.141 | 2.98 | 0.959 | 2.585 | 0.93 | 2.095 | 1.655 | 0.249 | −0.396 | 0.799 | 1.183 |
| HSPA5BP | 693776II [symbol] | −0.113 | −0.164 | 2.249 | 0 | 0.123 | −0.563 | 0.579 | 0 | −0.44 | 3.9 | −0.862 | −0.288 | 1.403 | 0.38 | −0.241 |
| HSPC016 | 688206II [symbol] | −0.3015 | −0.0045 | −0.3675 | 0.3925 | −0.1875 | −0.0305 | −0.0795 | −0.3765 | 0.05 | 0.4525 | −0.6285 | −0.5465 | 0.0035 | −0.0825 | −0.5175 |
| HSPC138 | 696635II [symbol] | −0.988 | −1.676 | 0.311 | −1.31 | −1.486 | −1.3 | −1.714 | −0.664 | 0.18 | 0.371 | −1.121 | −1.223 | −0.053 | −1.03 | −0.622 |
| HSPC148 | 687877II [symbol] | 0.153 | −0.014 | 0.505 | −0.071 | 0.394 | 0.266 | 1.243 | −0.038 | −0.04 | 0.203 | −0.388 | 0.386 | −0.522 | −0.356 | −0.001 |
| HSFC150 | 692010II [symbol] | 0.3565 | 0.7525 | 1.144 | −0.1855 | −0.4225 | −0.7435 | 1.996 | 2.565 | −0.96 | 0.7315 | −0.5515 | 1.716 | 1.145 | −0.1965 | 1.101 |
| HSPC183 | 691987II [symbol] | 0.044 | 0.126 | 1.421 | −0.69 | −0.588 | 0.02 | 0.479 | 0.872 | 0.12 | 0.014 | −0.424 | 0.278 | 0.62 | 0.928 | 0.607 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| HSPC268 | 693184II [symbol] | −0.685 | 0.507 | 0.675 | 1.048 | 0.909 | 0.363 | 0.464 | 0.038 | 0.16 | −0.354 | 0.149 | 0.479 | −0.202 | −0.969 | −1.231 |
| HSU7927 [symbol] | 693843II [symbol] | −0.011 | 0.331 | 1.21 | −0.189 | 0.292 | 0.161 | 0.115 | 0.533 | 0.19 | −0.235 | 0.158 | 0.103 | 0.262 | 0.101 | 0.244 |
| HTATIP2 | 693716II [symbol] | −0.277 | −0.384 | 1.4 | −0.661 | 0.109 | −0.706 | 1.926 | 1.272 | −0.75 | 0.184 | −0.025 | 0.661 | −0.213 | −0.368 | 0.956 |
| ICMT | 681046II [symbol] | −0.1455 | 0.0165 | 0.2035 | 0.2745 | 0.2515 | −0.1945 | 0.2885 | 1.096 | −0.13 | 0.4425 | −0.0725 | 0.6615 | −0.1535 | −0.0985 | −0.0485 |
| ICT1 | 683297II [symbol] | −0.1065 | 0.9805 | −0.1345 | −0.2645 | 0.1525 | 0.4905 | −0.2865 | 0.3085 | 0.04 | −0.0115 | −0.1176 | 0.2985 | −0.0025 | −0.8895 | 0.2175 |
| ID4 | 685578II [symbol] | 0.0015 | −0.1335 | −0.2125 | 0.0195 | 0.1555 | 0.4445 | 0.0345 | −0.3595 | 1.52 | −0.1285 | 0.2245 | 0.0825 | −0.2615 | −0.4735 | −0.5225 |
| IDH2 | 681706II [symbol] | 1.011 | 2.896 | −0.245 | −0.488 | −0.417 | −1.043 | 0.049 | 1.076 | −1.38 | 0.366 | 0.507 | −0.224 | −0.049 | 1.284 | 1.076 |
| ID5 | 696420II [symbol] | −0.655 | −0.611 | 3.267 | −0.293 | −0.196 | −0.591 | −0.778 | 2.454 | 0.17 | 3.693 | −1.045 | 2.07 | −0.573 | −0.786 | 1.995 |
| IFI30 | 695270II [symbol] | 2.959 | 0.946 | 0.483 | 1.649 | 1.02 | 2.389 | 1.88 | 1.139 | −1.15 | 1.845 | 2.411 | 1.696 | 2.162 | 1.786 | 2.044 |
| IFI44 | 681212II [symbol] | −0.415 | 0.011 | −0.458 | 0.138 | 0.088 | 0.267 | −0.011 | −0.431 | −0.57 | −0.642 | 0.446 | −0.064 | −0.42 | −0.477 | 0.216 |
| IFIT5 | 684503II [symbol] | −0.7882 | −0.2242 | 1.203 | −0.3572 | −0.1022 | 0.3238 | 0.2988 | 1.461 | −0.18 | −0.9102 | 0.5518 | 0.8188 | 0.0868 | 0.1008 | 0.7148 |
| IFITM1 | 690666II [symbol] | −1.025 | −1.499 | −2.707 | −0.794 | 0.816 | 0.117 | 0.904 | −1.225 | 0.08 | −0.801 | 0.867 | −1.564 | −0.337 | 0.458 | 0.026 |
| IGBP1 | 697847II [symbol] | −0.066 | 0.564 | −0.264 | 0.308 | 0.345 | −0.035 | −0.948 | −0.72 | 0.53 | −0.545 | 0.122 | −1.594 | −0.346 | −0.284 | −0.474 |
| IGFALS | 692644II [symbol] | −0.183 | 0.983 | −0.133 | 1.914 | 3.601 | 1.034 | −0.088 | −0.432 | 0.75 | 0.067 | 0.768 | 0.189 | −0.2 | −0.132 | −1.043 |
| IGFBP2 | 689980II [symbol] | 1.051 | −0.3605 | 1.671 | 2.463 | −3.084 | 0.8445 | 0.7355 | −2.979 | 0.61 | −1.205 | 1.282 | −1.219 | 1.408 | 0.0445 | −2.984 |
| IGHG1 | 682030II [symbol] | 3.133 | −0.055 | −0.544 | −0.61 | −0.161 | −0.753 | 0.733 | −0.152 | −0.25 | −0.362 | −0.371 | 0.491 | −0.202 | −0.001 | 2.804 |
| IGHG1 | 696457II [symbol] | 3.923 | −0.046 | 0.101 | 0.48 | 0.363 | 0.57 | 1.008 | 0.134 | −0.41 | 0.279 | −1.345 | 1.534 | 0.373 | 0.499 | 2.828 |
| IGJ | 687674II [symbol] | 1.609 | −1.75 | −3.089 | −0.2245 | 0.1715 | −2.747 | 1.752 | −2.842 | 1.24 | −2.373 | 0.0355 | −0.0355 | 0.1475 | −0.3085 | 2.217 |
| IGKC | 690921II [symbol] | 3.578 | 1.387 | −1.336 | −0.389 | 2.14 | −0.683 | 1.006 | −1.017 | −0.86 | −1.099 | 0.2 | 2.108 | 0.245 | 0.889 | 3.312 |
| IGKC | 691031II [symbol] | 3.638 | 0.323 | −0.229 | −0.201 | 1.034 | −0.588 | 0.059 | −0.506 | −1.07 | −0.186 | −0.086 | 0.822 | 1.522 | 0.589 | 3.51 |
| IGLC2 | 682716II [symbol] | 2.504 | −0.0085 | −0.2925 | 0.1515 | 1.34 | −0.3555 | 0.4485 | −0.3245 | −0.67 | −0.2155 | −0.0555 | 0.0385 | 1.098 | 0.0425 | 2.607 |
| IGLL1 | 691836II [symbol] | 3.265 | −0.7 | −1.323 | −0.611 | 2.352 | −1.266 | 0.118 | −1.459 | −0.77 | −1.325 | −0.368 | 0.506 | 1.241 | 0.353 | 2.849 |
| IL13RA1 | 697838II [symbol] | 0.388 | −0.016 | −1.141 | 0.081 | −0.487 | 0.34 | −0.282 | 1.014 | 0.01 | −0.087 | 0.234 | −0.756 | 0.368 | 1.372 | −0.162 |
| IL17B | 685612II [symbol] | −0.1405 | −1.008 | 0.4015 | 0.4075 | −0.5225 | −0.2695 | 3.964 | −0.4125 | 1.78 | −0.1655 | −0.4385 | −0.1565 | −0.0515 | 0.0545 | 0.0915 |
| IL23A | 690046II [symbol] | 0.502 | 0.425 | −0.279 | −0.366 | 0.275 | 0.093 | 0.166 | 1.926 | −0.27 | −0.078 | 0.051 | 0.285 | 0.255 | −0.621 | 0.476 |
| IL6ST | 690686II [symbol] | −0.9835 | −0.2695 | −1.137 | 1.26 | −0.0175 | 0.4245 | −1.192 | −0.5085 | 0.07 | −0.7495 | 1.207 | −1.968 | −0.6415 | −0.7635 | −0.3975 |
| ILF2 | 681204II [symbol] | 2.365 | 0.499 | 0.852 | 0.868 | 0.812 | 0.496 | 3.258 | 1.396 | −0.03 | 0.333 | 2.625 | 3.651 | 1.526 | 0.943 | 2.414 |
| IMPA2 | 690416II [symbol] | −0.478 | −0.783 | 1.851 | −1.092 | −0.703 | 1.033 | 2.183 | 2.212 | −0.15 | 0.745 | −0.209 | 1.238 | −0.736 | −0.568 | 1.159 |
| ING1L | 681976II [symbol] | −0.8195 | −0.0065 | −0.7155 | −0.4475 | −0.3835 | −0.0005 | 0.0245 | 0.5845 | 0.41 | 0.8775 | 0.6275 | 0.3815 | 0.2435 | −0.8845 | −0.2375 |
| INHBB | 695995II [symbol] | 3.552 | 3.98 | 0.816 | 3.615 | 0.336 | 3.212 | 0.798 | −0.068 | 0.9 | 1.104 | 1.906 | −0.19 | 0.519 | 3.446 | 0.83 |
| INPP4B | 685012II [symbol] | 0.244 | 0.149 | −2.689 | 0.958 | −0.37 | 1.27 | −0.55 | −2.631 | 1.27 | −2.583 | 0.097 | −2.614 | 0.766 | −2.116 | −2.667 |
| INSM1 | 693435II [symbol] | −0.174 | 0.143 | −0.397 | 6.039 | −0.104 | −0.521 | 0.808 | 0.064 | −0.07 | −0.315 | −0.064 | −0.332 | 4.575 | −0.216 | 0.937 |
| IQCA | 692963II [symbol] | 0.235 | 0.897 | 2.335 | 0.767 | 0.497 | 0.298 | −0.176 | −0.357 | 1.38 | 2.649 | 1.704 | 3.684 | 0.113 | 0.801 | −0.394 |
| IQCE | 687430II [symbol] | 0.2445 | 0.0025 | −0.0025 | 1.184 | 1.011 | −0.0195 | −1.489 | −1.575 | 0.9 | −0.7185 | −0.0345 | 0.0925 | −0.3635 | 0.5055 | 0.4285 |
| IQGAP2 | 697172II [symbol] | 0.2585 | 0.5295 | −0.2135 | 0.3825 | 0.2655 | 0.1985 | −0.0095 | −0.5235 | 0.57 | −0.4335 | −0.3335 | 0.3665 | 0.0395 | −0.2596 | −1.291 |
| IRF4 | 690581II [symbol] | 1.926 | −1.039 | −0.7285 | −0.4935 | 0.3585 | −0.3125 | 0.7325 | −0.7205 | −0.09 | −0.3985 | −0.9005 | 0.0825 | 0.2695 | −0.1615 | 1.929 |
| IRTA2 | 686673II [symbol] | 2.215 | 0.45 | −0.631 | −0.761 | 3.027 | −1.187 | 1.554 | −0.74 | −0.48 | −0.701 | −0.313 | 0.337 | 0.623 | −0.031 | 3.138 |
| IRX4 | 682616II [symbol] | 2.193 | 1.646 | 2.868 | −0.81 | −0.543 | −1.482 | −2.658 | −1.72 | 3.04 | 3.826 | 0.293 | 0.861 | 1.374 | −0.63 | −2.193 |
| ISG20 | 689588II [symbol] | 0.1105 | −0.9835 | −0.1095 | −0.1085 | −0.3325 | 0.5005 | 0.1995 | −0.4055 | −0.89 | 0.3275 | 0.0005 | −0.3885 | −0.2335 | −0.2485 | −0.0505 |
| ISGF3G | 684521II [symbol] | 1.039 | 0.864 | 1.799 | 0.231 | 1.139 | 0.497 | 0.671 | 2.916 | −0.77 | 0.428 | 0.955 | 0.732 | 1.169 | 0.813 | 2.833 |
| ITGB4 | 692936II [symbol] | 0.061 | 1.061 | 0.287 | 4.251 | 1.98 | 0.12 | 0.439 | 0.196 | 1.61 | 0.094 | 1.577 | 0.323 | 0.016 | 0.153 | −0.032 |
| ITPR1 | 690279II [symbol] | −0.4435 | 1.577 | −1.299 | 1.653 | 2.336 | −0.0515 | −0.7895 | −1.01 | 1.24 | −1.658 | 1.153 | −0.8815 | 0.2125 | −0.2475 | −0.1095 |
| JARID1A | 694293II [symbol] | 0.248 | 0.974 | 2.177 | 0.334 | 0.397 | 0.636 | 0.19 | 0.964 | −0.16 | 0.253 | 0.168 | 0.56 | 0.01 | −0.04 | 1.125 |
| KARCA1 | 692006II [symbol] | −0.404 | 0.264 | 0.558 | 1.382 | 0.976 | 0.537 | −3.487 | −2.698 | 0.63 | −2.575 | −0.321 | 0.673 | 0.517 | −0.212 | 0.056 |
| KATNA1 | 692605II [symbol] | 0.171 | −1.806 | 1.005 | −0.498 | −0.892 | −0.262 | 0.117 | 0.018 | 0.34 | −0.116 | 0.137 | 0.397 | −0.265 | −0.214 | 0.352 |
| KCNC2 | 686570II [symbol] | −0.2385 | −0.0915 | −0.4035 | 3.885 | −0.4365 | 0.4915 | 0.1055 | −0.3105 | 0.1 | 0.1135 | 2.384 | 0.2595 | 0.2635 | 0.1155 | −0.5285 |
| KCND2 | 683134II [symbol] | 0.762 | −0.167 | −0.3252 | −0.05 | −0.541 | 0.339 | −0.088 | −0.836 | −0.02 | −0.255 | 1.134 | −0.318 | 0.868 | −0.355 | −0.145 |
| KCNK4 | 691761II [symbol] | 1.143 | 0.253 | −0.153 | −0.386 | 0.087 | 0.16 | 0 | −0.265 | 0.02 | 1.982 | 0.536 | 0.161 | −0.17 | −0.299 | 0.485 |
| KCNN4 | 692275II [symbol] | 0.5145 | −0.9385 | −1.058 | 0.7795 | 0.6255 | −0.2625 | −0.2745 | 0.1065 | 0.57 | −0.1015 | −0.0075 | 0.3265 | 0.3575 | −0.1335 | −0.0635 |
| KCNS3 | 681093II [symbol] | 0.8105 | −1.818 | −0.7305 | −0.0395 | 0.6445 | 0.4795 | 0.5755 | −0.3735 | 0.82 | −2.35 | −0.5925 | −1.057 | −1.337 | 1.212 | −0.5235 |
| KCTD15 | 689199II [symbol] | −0.0095 | −0.7915 | 1.228 | 0.2995 | −1.781 | 0.5395 | 0.3695 | −0.0835 | 0.65 | −1.072 | −0.7435 | 0.9315 | −1.146 | −0.2765 | −1.165 |
| KCTD3 | 686312II [symbol] | −0.424 | 1.102 | −0.163 | 1.277 | 0.709 | −0.06 | −0.369 | −0.074 | 0.61 | −1.981 | 0.009 | −0.009 | −0.919 | 0.669 | −0.117 |
| KDELR2 | 697248II [symbol] | 0.494 | 0.704 | −1.632 | −0.157 | −0.383 | −1.782 | −1.093 | −0.373 | −0.55 | 0.99 | −0.692 | 0.038 | −0.915 | −0.522 | 0.618 |
| KIAA0020 | 696703II [symbol] | −1.209 | −1.48 | −0.351 | −1.059 | −1.203 | −1.013 | 0.225 | −0.056 | 0.4 | 0.307 | −1.334 | −0.642 | −0.593 | −0.322 | 0.592 |
| KIAA0101 | 691153II [symbol] | 0.4855 | 0.6905 | −0.5105 | −0.3015 | −0.6585 | 0.2295 | 0.2025 | 1.244 | −1.26 | 2.724 | 0.0795 | 0.3455 | 0.9325 | −0.5195 | 0.3875 |
| KIAA0153 | 694669II [symbol] | 0.855 | 0.573 | −0.13 | 0.496 | 0.467 | 0.378 | 0.77 | 1.336 | −0.25 | 0.765 | −0.624 | 0.749 | 1.024 | 0.285 | 0.087 |
| KIAA0182 | 691550II [symbol] | 0.6547 | 1.988 | −0.3683 | −0.1133 | 1.845 | −0.2483 | −0.1743 | −0.4393 | −0.12 | 0.4647 | −0.4633 | −1.001 | 0.2877 | −0.6973 | −2.014 |
| KIAA0196 | 685973II [symbol] | −0.4475 | 1.698 | −0.2505 | −0.0145 | 0.3505 | 1.605 | 0.5125 | 0.8755 | 0.05 | −0.5915 | 0.5945 | 0.4435 | −0.0175 | 1.307 | 0.1785 |
| KIAA0241 | 692587II [symbol] | −0.179 | −0.195 | −0.806 | −0.86 | −1.106 | −0.704 | −0.562 | −0.489 | −0.58 | 0.693 | −0.644 | −1.279 | −0.2 | −0.266 | −0.414 |
| KIAA0251 | 682227II [symbol] | −0.6855 | 0.9995 | 0.1235 | −0.2255 | 0.6605 | 0.4725 | −0.0365 | 0.1385 | −0.07 | −0.1865 | 0.1205 | 0.5415 | −0.0435 | 0.2475 | −0.1345 |
| KIAA0274 | 683166II [symbol] | 0.2755 | −0.0275 | 0.2265 | 0.5675 | 0.6465 | 1.736 | −0.3915 | −0.8155 | 0.05 | −1.133 | 0.1025 | −0.5295 | 0.6115 | 0.3595 | 0.3635 |
| KIAA0515 | 688133II [symbol] | 0.042 | 0.236 | −0.303 | 0.249 | 0.235 | −0.098 | 0.93 | −0.173 | −0.11 | −0.21 | 0.001 | 0.452 | −0.695 | −0.153 | −0.276 |
| KIAA0582 | 697645II [symbol] | 0.127 | 1.104 | 0.375 | 1.435 | 1.696 | 2.661 | 0.262 | 0.554 | 0.59 | −0.244 | 1.006 | 0.129 | −0.122 | 0.203 | 0.311 |
| KIAA0650 | 684609II [symbol] | −0.2685 | −0.5445 | 0.6935 | −0.7475 | −0.0575 | −0.3095 | 1.188 | 0.6515 | 0.43 | 0.4685 | −0.6895 | −0.3975 | −0.4585 | 0.1945 | 1.322 |
| KIAA0703 | 684139II [symbol] | 1.224 | 0.8725 | −0.1475 | 0.7515 | 0.5255 | −0.4165 | 0.1585 | 1.706 | −0.35 | −0.4555 | −0.5825 | −0.7745 | 0.1625 | 0.0835 | −0.6505 |
| KIAA0830 | 692561II [symbol] | 0.823 | −0.74 | 1.463 | 1.781 | 0.021 | 1.094 | 1.244 | 0.354 | 0.18 | −0.67 | 0.475 | −0.681 | −0.282 | 0.659 | 1.534 |
| KIAA0980 | 686300II [symbol] | −1.562 | 0.707 | 1.301 | −1.309 | 0.049 | 0.385 | −0.27 | 0.438 | 0.52 | −2.163 | −0.38 | 0.12 | −0.042 | −0.746 | −0.138 |
| KIAA1324 | 690969II [symbol] | −0.7208 | 0.1552 | −1.932 | 1.868 | 0.3792 | 0.0553 | −3.935 | −3.311 | 0.71 | −4.858 | 0.1642 | −0.8498 | −1.489 | −0.6358 | −4.158 |
| KIAA1363 | 696389II [symbol] | 0.781 | −0.487 | −1.503 | 0.01 | −0.521 | 0.045 | −1.608 | 0.848 | −0.63 | −1.056 | −0.281 | −0.596 | −0.202 | 0.025 | 0.034 |
| KIAA1683 | 684555II [symbol] | −0.0145 | −0.6375 | −0.2575 | 0.5655 | 0.5975 | 0.0425 | −0.2205 | −0.3065 | 0.65 | −1.129 | −0.3655 | −0.3815 | −0.1785 | −0.2755 | −0.5565 |
| KIF13B | 689768II [symbol] | 0.089 | −1.391 | −0.88 | 1.909 | −0.518 | 2.473 | −1.409 | −1.592 | 1.48 | −3.069 | 0.558 | −1.683 | −0.383 | 0.92 | 0.32 |
| KIF24 | 692033II [symbol] | −0.132 | −1.19 | 0.448 | 0.005 | −0.215 | 0.275 | 0.584 | 0.466 | 0.06 | −0.8 | −0.121 | 0.106 | 0.155 | 0.354 | −0.183 |
| KIN | 687329II [symbol] | 0.047 | −0.625 | 0.397 | −0.01 | −0.436 | −0.335 | −0.377 | 0.73 | 0.11 | 0.121 | −0.569 | 0.134 | −0.104 | −0.226 | 0.406 |
| KIRREL3 | 693623II [symbol] | 0.428 | 0.312 | 0.667 | −0.344 | 1.61 | −0.443 | 0.134 | −0.011 | −0.04 | 0.058 | 0.318 | 0.35 | −0.228 | −0.237 | 0.175 |
| KIT | 692519II [symbol] | −1.056 | −0.687 | 0.79 | −1.028 | 0.248 | 1.364 | −0.88 | 0.142 | 0.71 | −0.995 | 0.61 | 2.206 | −0.778 | 0.625 | 3.495 |
| KLK5 | 693542II [symbol] | 0.836 | 0.758 | 3.22 | 0.591 | 0.59 | 0.451 | 0.295 | 1.786 | 1.46 | 0.259 | 0.653 | 3.426 | 0.235 | 0.533 | 0.589 |
| KLK6 | 684796II [symbol] | 1.944 | −0.2187 | 3.834 | −0.6597 | −0.0168 | −0.8257 | −1.603 | 3.255 | 1.98 | 3.004 | −0.5817 | 1.402 | 0.7163 | 0.0623 | −0.1017 |
| KLK7 | 695787II [symbol] | 0.3 | −1.102 | 3.966 | 0.841 | −1.102 | 0.001 | 0.181 | 3.34 | 1.41 | 1.517 | −0.04 | 2.373 | 0.276 | 0.078 | 0.017 |
| KLK8 | 688892II [symbol] | −0.0975 | −0.2495 | 2.83 | −0.0195 | −0.5245 | 1.291 | −0.5805 | 3.935 | 2.05 | 3.532 | −0.2785 | 3.581 | −0.5235 | −0.2165 | −0.7555 |
| KNTC2 | 694720II [symbol] | −1.874 | −2.503 | −1.398 | −2.773 | −1.736 | −2.889 | 0.698 | −0.515 | −1.24 | −0.283 | −2.102 | −1.284 | −1.631 | −2.131 | −0.085 |
| KPNA2 | 689153II [symbol] | 0.5265 | 0.8915 | 0.1995 | −0.5265 | −0.4805 | −0.4745 | 1.05 | 2.355 | −0.45 | 1.114 | −0.4625 | 0.0685 | 0.6635 | 0.4785 | 0.7065 |
| KREMEN [symbol] | 687365II [symbol] | 0.96 | −1.542 | 2.757 | −0.079 | −1.246 | 1.13 | 0.043 | 1.82 | −0.29 | 2.234 | −0.664 | 0 | 1.304 | 0.955 | −0.214 |
| KRT10 | 694359II [symbol] | −0.122 | −1.572 | −2.49 | −0.716 | −1.63 | −1.313 | −0.944 | −0.556 | 0.08 | −0.862 | −0.997 | −0.654 | −0.81 | −1.391 | −0.895 |
| KRT13 | 696433II [symbol] | 2.033 | 0.125 | −0.025 | −0.107 | −0.228 | −0.559 | 0.228 | 1.244 | 1.45 | −0.829 | −0.425 | 2.748 | 0.194 | 0.249 | 2.377 |
| KRT15 | 694764II [symbol] | 0.111 | 1.568 | 0.26 | 0.175 | 0.438 | 0.098 | −0.559 | 0.532 | 1.48 | −0.167 | −0.089 | 0.491 | 0.369 | −0.994 | −0.131 |
| KRT17 | 690706II [symbol] | 0.672 | −0.705 | 0.059 | −0.041 | −1.159 | −0.99 | −0.2 | 0.591 | 1.51 | −0.172 | −1.198 | −0.289 | 0.492 | 0.308 | 1.975 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| KRT18 | 689283II [symbol] | 0.865 | 2.4 | −1.884 | −0.061 | 0.312 | 0.611 | −1.874 | −2.003 | −0.06 | 1 | 0.31 | −1.487 | −0.078 | 0.13 | −2.135 |
| KRT23 | 689793II [symbol] | 0.396 | −2.402 | −0.093 | 0.437 | −3.047 | −0.886 | 2.906 | 0.693 | −0.15 | 0.294 | −2.513 | 3.46 | 0.201 | −1.105 | 1.815 |
| KRT6B | 696954II [symbol] | 4.848 | 2.002 | 0.16 | 1.751 | 0.356 | 0.841 | 1.186 | 4.578 | 2.09 | −1.439 | 1.33 | 2.001 | 1.722 | 2.662 | 4.762 |
| KRTAP3-3 | 684073II [symbol] | −0.0895 | −0.7195 | −0.0445 | 6.526 | −0.0005 | 0.1985 | −0.1445 | 0.0485 | 0.12 | 0.0795 | −0.0295 | −0.0455 | 0.0615 | −0.0635 | −0.1505 |
| KRTHA7 | 683234II [symbol] | −0.3275 | 2.165 | −0.1776 | 0.0065 | −0.2075 | 0.2055 | 0.1075 | −0.0285 | −0.09 | −0.2355 | 0.9615 | −0.0115 | 0.9075 | −0.1405 | 0.0165 |
| LAMA3 | 689785II [symbol] | −0.4585 | −0.0555 | −0.7935 | 0.8125 | −0.6985 | 1.121 | −0.4525 | −0.7865 | 1.58 | −1.073 | 1.64 | −0.2535 | −0.6365 | −0.6425 | −0.6515 |
| LAMB3 | 764691II [symbol] | −0.889 | −2.46 | 0.534 | −1.473 | −1.52 | −2.45 | −2.149 | 0.063 | 2.61 | −1.501 | −2.46 | −1.75 | 0.96 | −1.49 | −0.326 |
| LASS2 | 695705II [symbol] | 0.699 | 0.62 | −0.655 | 0.178 | −1.464 | −0.33 | −0.61 | −0.899 | −0.1 | −1.651 | 0.442 | −2.588 | 1.192 | 0.931 | 0.876 |
| LAX | 682690II [symbol] | 1.475 | −0.4945 | −0.1255 | −0.4845 | −0.0945 | 0.0625 | 0.3175 | −0.3555 | −0.27 | −0.5975 | 0.2645 | −0.0475 | 0.0585 | −0.1565 | 1.929 |
| LBR | 681309II [symbol] | 0.0268 | −0.9573 | 1.065 | −0.9203 | −1.236 | −1.638 | 1.534 | 1.795 | −0.36 | 1.369 | −0.6453 | 0.7137 | 1.009 | 0.5807 | 2.137 |
| LCE1B | 693796II [symbol] | −1.215 | −0.114 | −2.003 | −1.614 | 0.205 | −0.104 | −1.241 | 0.5 | 0.56 | −0.929 | 1.415 | 0.684 | −0.832 | −0.62 | −1.065 |
| LDHB | 683774II [symbol] | −1.295 | −1.465 | 3.404 | −1.492 | 1.548 | −1.471 | 0.3055 | 0.7935 | 0.62 | 2.133 | −0.3705 | 0.7535 | −0.7505 | 0.7625 | 2.881 |
| LDOC1L | 692299II [symbol] | −0.734 | −0.242 | −0.156 | −0.475 | −0.184 | −0.123 | −1.184 | −0.031 | 0.58 | −0.865 | 0.712 | −0.61 | 0.769 | 0.007 | 0.179 |
| LETMD1 | 682701II [symbol] | −0.37 | 0.269 | −0.171 | 0.481 | 0.234 | 0.169 | −0.376 | −0.335 | 0.46 | −0.821 | 0.092 | −0.193 | −0.525 | 0.42 | −0.171 |
| LFNG | 683275II [symbol] | 0.9885 | 1.414 | −2.457 | 1.047 | −1.213 | −0.5685 | −1.195 | −2.95 | 0.74 | −0.7425 | −0.0455 | −2.063 | −0.2635 | −1.933 | −1.333 |
| LGALS2 | 696910II [symbol] | 1.322 | −1.677 | −1.629 | −0.856 | −1.07 | −1.665 | 0.964 | −0.575 | 0.77 | −1.35 | −1.221 | 0.25 | 1.032 | −0.606 | 1.232 |
| LGTN | 694133II [symbol] | −0.111 | −0.03 | −0.33 | 0.249 | 0.683 | 0.066 | 0.239 | 0.687 | 0.24 | −1.937 | −0.594 | 0.758 | 0.254 | 0.267 | −0.16 |
| LNX | 686766II [symbol] | 1.059 | 0.4855 | 0.4645 | 1.022 | 1.473 | 0.3195 | −1.533 | 0.3675 | −0.22 | −1.362 | 0.0895 | −0.7715 | 0.1295 | −0.4795 | −2.09 |
| LOC11247 | 687612II [symbol] | −1.258 | −2.96 | −1.331 | −2.643 | −3.366 | −2.197 | −0.72 | −0.73 | 1.13 | 0.282 | −2.465 | −1.896 | −1.224 | −2.029 | −0.847 |
| LOC11492 | 691804II [symbol] | −0.175 | 0.597 | 0.868 | 0.51 | 0.049 | 0.984 | −0.491 | −0.767 | 0.12 | −0.313 | −0.157 | 1.117 | 0.06 | −0.42 | 0.471 |
| LOC11497 | 691267II [symbol] | 0.236 | −0.17 | 2.071 | 0.078 | −0.375 | 0.024 | 0.801 | −0.44 | −0.3 | 0.476 | 0.28 | −0.102 | 0.264 | −1.102 | 0.849 |
| LOC11621 | 686452II [symbol] | −0.1075 | −0.2555 | 0.3685 | 0.3075 | 0.4955 | 0.1545 | 0.6275 | 0.9725 | 0.26 | −0.0965 | −0.7115 | −0.5035 | −0.6605 | 0.5605 | −0.3195 |
| LOC11644 | 695259II [symbol] | 0.464 | 0.334 | 0.558 | 0.591 | 0.467 | 0.307 | 0.381 | 0.262 | 0.88 | 0.282 | 0.238 | 0.301 | 0.586 | 0.329 | 2.256 |
| LOC12022 | 693599II [symbol] | 0.835 | −3.311 | −2.524 | −1.893 | −2.913 | 0.532 | −3.381 | −3.375 | 1.8 | −2.136 | 1.039 | −2.841 | −3.274 | 0.439 | −3.212 |
| LOC12422 | 694408II [symbol] | 3.766 | 1.817 | 0.835 | 3.368 | 2.587 | 3.024 | −0.113 | 3.57 | 0.41 | −1.997 | 2.633 | 3.465 | 1.358 | 1.678 | 1.223 |
| LOC14654 | 687013II [symbol] | −0.5985 | 0.2605 | −0.2725 | 0.3755 | 0.2915 | 1.288 | −0.1475 | −1.324 | 0.16 | −1.003 | 0.1705 | 0.0875 | −0.2705 | −0.5585 | −0.4975 |
| LOC1550 [symbol] | 683455II [symbol] | −1.779 | 1.665 | −1.782 | −0.258 | 0.08 | −0.498 | −2.835 | −1.304 | 0.49 | −0.709 | 0.839 | −1.906 | 0.445 | −1.32 | −1.441 |
| LOC2018 [symbol] | 694158II [symbol] | 0.646 | 1.281 | −1.598 | 0.203 | 0.171 | 0.939 | −0.988 | −0.947 | 0.18 | −1.677 | 1.434 | −0.829 | 1.624 | 0.933 | −0.51 |
| LOC2539 [symbol] | 689291II [symbol] | −0.523 | 0.284 | 0.059 | 2.521 | 1.153 | 0.249 | −0.14 | 0.041 | −0.54 | −0.242 | −0.53 | 1.061 | 0.107 | −0.152 | −0.451 |
| LOC3399 [symbol] | 685165II [symbol] | 0.3735 | −0.7585 | −0.4426 | −1.112 | 1.15 | 2.048 | −0.3895 | −0.9455 | 0.98 | 0.6505 | −0.1555 | −0.3845 | 0.1395 | −0.1505 | −1.142 |
| LOC34817 | 686995II [symbol] | −0.474 | 0.429 | −0.284 | 0.082 | 0.588 | 0.529 | 0.692 | −0.086 | 0.67 | −0.084 | −0.104 | 1.142 | −0.008 | 0.372 | −0.095 |
| LOC4004 [symbol] | 683489II [symbol] | 0.3985 | 0.0945 | −2.407 | 0.9165 | 0.0185 | 0.7945 | −1.649 | −1.903 | 0.39 | −1.232 | 0.1115 | −2.059 | −0.5845 | 0.5885 | −2.711 |
| LOC4408 [symbol] | 696169II [symbol] | −2.027 | 0.559 | −0.565 | 0.713 | 1.485 | 1.421 | 0.598 | 0.458 | 0.21 | −0.108 | 0.871 | −0.125 | 0.605 | 1.241 | 0.845 |
| LOC4938 [symbol] | 684635II [symbol] | 0.086 | −0.249 | −1.804 | −1.374 | −0.045 | −1.804 | 0.436 | −0.894 | 0.33 | 0.656 | 0.464 | −0.962 | −0.09 | 0.862 | 0.606 |
| LOC51123 | 697791II [symbol] | −2.006 | 0.761 | −2.058 | −2.648 | −2.228 | −2.111 | −3.078 | −2.677 | −0.48 | −2.011 | −2.969 | −1.913 | −2.563 | −2.803 | −2.513 |
| LOC5116 [symbol] | 695670II [symbol] | −0.267 | 0.219 | −0.442 | 1.805 | 1.162 | 1.904 | −0.14 | −0.807 | 1.06 | −0.408 | 0.476 | 0.3 | −0.679 | −0.197 | 0.259 |
| LOC5132 [symbol] | 694469II [symbol] | −0.868 | −0.912 | −0.446 | −0.202 | −0.578 | −0.658 | −0.931 | −0.639 | −0.15 | −1.028 | −0.715 | −0.56 | 0.425 | −0.728 | 0.49 |
| LOC5176 [symbol] | 694904II [symbol] | 1.832 | 1.043 | −1.526 | 2.887 | 0.229 | 3.923 | 0.135 | −0.662 | 0.07 | 0.519 | 1.35 | −0.779 | 0.453 | 0.75 | −0.458 |
| LOC8155 [symbol] | 682974II [symbol] | −0.2025 | −0.0285 | 1.016 | −0.6145 | 0.3315 | −0.5895 | −0.2965 | 0.1125 | 0.55 | −0.5255 | 0.6365 | 0.5635 | −1.035 | −1.501 | 0.4315 |
| LOC8156 [symbol] | 695594II [symbol] | −1.783 | −0.221 | −0.732 | −2.165 | 0.041 | −1.494 | 5.466 | −0.453 | −0.22 | −1.365 | −1.607 | 0.347 | −0.857 | 1.823 | 2.221 |
| LOC9035 [symbol] | 690384II [symbol] | −0.8795 | 1.028 | −0.5715 | 0.8655 | 0.0795 | 0.5975 | −0.8935 | −0.6415 | −0.06 | 0.0945 | 1.271 | −1.502 | 0.1235 | −0.2425 | −0.3685 |
| LOC9070 [symbol] | 691788II [symbol] | 0.6155 | −1.251 | 0.0025 | 0.3845 | 1.405 | −0.9835 | 0.0135 | −1.148 | −0.44 | −1.585 | −0.3055 | 0.0895 | 0.2115 | 0.0116 | 0.4385 |
| LONP | 694535II [symbol] | 2.462 | 1.317 | −0.132 | 0.37 | 1.277 | 2.517 | 0.549 | 0.254 | 0.1 | 0.574 | 0.455 | 0.894 | 0.907 | 0.204 | −0.16 |
| LRAP | 694401II [symbol] | 0.062 | 0.041 | −0.454 | 0.476 | 1.011 | −0.378 | 0.083 | −0.326 | −1.35 | 0.092 | 0.462 | −1.181 | −0.134 | −0.796 | −0.086 |
| LRBA | 689006II [symbol] | −0.0643 | 0.8837 | 0.7387 | 0.9487 | −0.3023 | 0.4107 | −0.7713 | 0.1077 | 0.19 | −0.4043 | 0.0568 | 1.679 | 0.1807 | 0.0168 | 0.4397 |
| LRIG1 | 696908II [symbol] | 0.684 | 1.274 | 0.446 | 2.252 | 3.361 | 2.316 | −0.25 | 1.362 | 1.16 | −0.783 | 1.04 | 0.528 | 0.547 | 1.132 | 0.031 |
| LRP6 | 688139II [symbol] | −0.8435 | −0.0715 | 2.146 | −0.3445 | 0.6835 | −0.4485 | −0.9655 | 0.4775 | 0.34 | −1.372 | −0.3905 | 0.8455 | −0.7015 | −0.7305 | 1.53 |
| LRP8 | 684430II [symbol] | 1.342 | −0.216 | 2.429 | −1.389 | 0.071 | −0.548 | 2.225 | 2.508 | −1.2 | −0.027 | −0.895 | 2.233 | 1.215 | 0.293 | 1.685 |
| LRRC17 | 689237II [symbol] | 0.3715 | 0.4165 | 0.4245 | 0.4845 | 0.4585 | −0.3455 | 0.1065 | −0.2305 | 0.65 | −0.6695 | −0.4055 | −0.5555 | 0.3826 | −0.2585 | 0.6375 |
| LRRC2 | 696221II [symbol] | 0.98 | 0.407 | 0.11 | 1.081 | −0.019 | 1.626 | −0.33 | 0.395 | 0.06 | 0.342 | 1.363 | −0.501 | 0.823 | 0.954 | 0.777 |
| LSM1 | 693186II [symbol] | −0.52 | 0.335 | 0.387 | −0.215 | −0.75 | 0.64 | −0.05 | −0.211 | 0.37 | 0.197 | −0.416 | 0.635 | 0.071 | −0.571 | 0.089 |
| LSM8 | 689434II [symbol] | −0.276 | −0.314 | 0.303 | 0.15 | −0.035 | −0.482 | 0.368 | 0.589 | 0.04 | −0.199 | −0.271 | −0.857 | −0.061 | −0.273 | 0.04 |
| LTB | 695788II [symbol] | 3.347 | 0.322 | 3.221 | 1.619 | 1.604 | 1.236 | 4.626 | 3.237 | −0.16 | 0.963 | 1.77 | 1.96 | 2.547 | 1.563 | 3.895 |
| LY6D | 688248II [symbol] | 1.027 | 0.078 | 1.451 | −0.348 | 0.058 | −0.459 | 2.067 | 1.756 | 0.06 | 0.594 | −0.018 | −0.024 | 0.826 | −0.436 | 2.971 |
| LYN | 690653II [symbol] | −0.072 | −0.558 | −1.158 | −0.624 | 0.096 | 0.209 | 1.515 | 1.61 | −0.72 | 1.339 | 0.206 | 1.239 | −0.252 | −0.336 | 1.327 |
| MAD2L1 | 684649II [symbol] | 0 | 0.51 | 0.808 | −0.599 | 0 | −0.92 | 0.919 | 1.566 | −0.84 | 2.496 | −0.08 | 0.54 | 1.5 | 0.25 | 2.472 |
| MADP-1 | 687178II [symbol] | −0.1515 | 0.3195 | −0.5105 | 0.2695 | 0.2675 | 0.1105 | −0.9015 | −0.3885 | 0.16 | −0.2235 | 0.3035 | 0.2785 | −0.0195 | −0.6115 | 0.4235 |
| MAGEA12 | 696395II [symbol] | −0.962 | −0.71 | −2.984 | −0.308 | −1.226 | −1.284 | −2.599 | −3.087 | −0.12 | −3.026 | −0.54 | −2.654 | −1.742 | 0.589 | −1.661 |
| MAGEA2 | 696763II [symbol] | −3.019 | 1.304 | −4.248 | −3.628 | −2.728 | −2.588 | −3.868 | −3.765 | −0.14 | −2.961 | −3.217 | −3.904 | −3.617 | −3.346 | −3.427 |
| MAGEA3 | 689265II [symbol] | −1.154 | 0.0955 | −1.544 | 1.409 | 0.6485 | 0.6065 | −0.4895 | −0.8495 | −0.04 | 0.3715 | 0.0165 | 0.8915 | 0.3585 | 0.3495 | −2.116 |
| MAGEA4 | 690918II [symbol] | 0.5115 | 4.455 | −0.3795 | −0.3655 | −1.126 | −0.1535 | −0.1895 | 0.3765 | 0.02 | 1.04 | −0.5555 | 0.0295 | −0.0455 | −0.5785 | 0.2755 |
| MAGED4 | 683069II [symbol] | −0.0815 | 0.1475 | 2.092 | −0.5325 | 0.1685 | −0.3125 | −0.6485 | −0.4135 | 0.15 | 2.023 | −0.4495 | 0.0745 | 2.831 | −0.0065 | −0.8945 |
| MAGI1 | 688247II [symbol] | 0.0075 | −2.313 | 2.145 | 2.706 | 0.3435 | −0.9205 | 0.9085 | 2.77 | 0.39 | −2.205 | −0.8445 | 1.629 | −0.0455 | −1.157 | 1.609 |
| MAGI-3 | 692838II [symbol] | 0.814 | 1.005 | 0.2 | 1.404 | 0.286 | 1.506 | 0.838 | 0.648 | 0.63 | −0.108 | 1.118 | 0.138 | 1.142 | 0.5539 | −0.013 |
| MAL2 | 683958II [symbol] | −0.442 | 1.323 | 0.649 | −0.09 | −0.757 | 0.269 | 0.867 | 1.108 | −0.42 | −0.744 | −0.734 | 1.807 | 0.823 | 1.251 | 0.874 |
| MAN2B1 | 690298II [symbol] | −0.001 | 1.436 | −0.626 | 0.208 | 0.476 | 0.303 | 0.588 | −0.174 | 0.32 | −0.842 | −0.431 | −1.06 | −1.03 | −1.14 | −0.69 |
| MAPK8IP [symbol] | 693222II [symbol] | 0.296 | 1.261 | −0.566 | 2.881 | 2.356 | 1.604 | 1.105 | 2.511 | 0.86 | 1.309 | 2.011 | 0.532 | 1.787 | 1.78 | 1.653 |
| MARCO | 691594II [symbol] | 2.873 | −0.1515 | 3.213 | −0.1485 | 0.0335 | −0.3605 | 1.812 | 3.011 | −0.95 | 1.703 | 0.0605 | 2.12 | 0.0585 | −0.2065 | 2.128 |
| MARLIN1 | 687609II [symbol] | 3.237 | 1.223 | −0.7675 | −0.5715 | 0.8475 | −0.6505 | 0.4555 | −0.7735 | −1.19 | −2.007 | −0.0755 | 0.0755 | 1.27 | −0.3475 | 1.441 |
| MASS1 | 687008II [symbol] | 1.843 | 0.1442 | −1.634 | 0.8152 | −1.15 | −0.2758 | −2.188 | −1.006 | 1.23 | −1.177 | −1.282 | −1.366 | 1.291 | 0.6132 | −0.7058 |
| MCM2 | 694711II [symbol] | −0.246 | −0.217 | −0.803 | −0.969 | −0.784 | −0.475 | −0.36 | −0.196 | −0.46 | −0.037 | −0.813 | −0.66 | −0.352 | −1.069 | 1.444 |
| MCM3 | 696856II [symbol] | −1.384 | −1.743 | −0.804 | −1.33 | −1.578 | −0.65 | −0.319 | −0.404 | 0.03 | 0.028 | −1.525 | −0.423 | −0.959 | −1.543 | −0.653 |
| MESP1 | 697846II [symbol] | −0.816 | 2.173 | 1.032 | −1.418 | 0.147 | 1.865 | 0.901 | 0.964 | −0.91 | −0.773 | −0.367 | 0.99 | −0.89 | 0.7484 | 0.934 |
| METRN | 691541II [symbol] | 0.4155 | 0.4545 | −1.302 | 0.3745 | 1.098 | 0.7915 | −0.2725 | −0.6895 | −0.04 | −0.9375 | −0.6035 | 1.414 | 0.8135 | 0.2135 | −2.579 |
| MFAP2 | 682204II [symbol] | −0.0785 | 0.3565 | 0.1085 | −0.8825 | −1.291 | −0.5225 | −0.4915 | −0.0595 | −0.52 | 0.0145 | 1.011 | 0.5215 | −0.9275 | −0.6155 | −0.3725 |
| MFGE8 | 695022II [symbol] | 0.234 | −0.529 | −0.187 | −0.347 | −0.229 | 0.119 | 0.3 | 1.759 | 0.04 | −0.146 | −0.094 | 0.513 | −0.041 | −0.229 | 1.221 |
| MGAT4B | 687726II [symbol] | 0.3065 | −1.071 | 1.023 | 0.1275 | −0.2975 | 0.2445 | 0.8745 | 0.8985 | −0.01 | 0.0095 | −0.2495 | 0.1505 | 0.3855 | 0.5885 | 0.6645 |
| MGC1091 | 690398II [symbol] | 0.1935 | −0.0245 | −0.9615 | 0.0335 | 0.4975 | −0.0245 | −0.3455 | −0.3555 | −0.16 | 0.7125 | −0.6215 | 0.3885 | −0.3885 | −0.1735 | −0.0155 |
| MGC1098 [symbol] | 687879II [symbol] | 0.6445 | −0.9315 | 1.584 | −2.072 | 0.9175 | −1.431 | −2.492 | 1.227 | −0.51 | −3.681 | −1.474 | 1.734 | −0.7535 | 0.4885 | 0.9115 |
| MGC1124 [symbol] | 680903II [symbol] | −0.4125 | 0.7745 | −0.4395 | 0.0995 | −0.4785 | 0.3725 | −1.113 | −1.398 | −0.59 | −2.697 | −0.1415 | −1.58 | −1.567 | 1.314 | 0.0825 |
| MGC1127 | 697307II [symbol] | −1.402 | −0.483 | −0.82 | −1.007 | −0.5068 | −0.325 | 0.283 | 0.583 | 0.42 | −1.668 | −0.091 | −0.376 | −0.164 | 0.809 | 0.359 |
| MGC1312 [symbol] | 695012II [symbol] | −0.341 | −0.685 | 0.094 | −0.487 | −1.026 | −0.058 | 0.006 | 0.172 | 0.17 | −0.451 | −0.347 | −0.388 | −1.166 | −0.663 | −0.492 |
| MGC1318 [symbol] | 687992II [symbol] | 0.0585 | −0.0675 | 2.386 | −0.2105 | −0.4675 | −0.0855 | 0.7265 | 1.489 | 0.08 | 0.6795 | 0.1505 | 1.252 | 0.1255 | −0.0838 | 1.267 |
| MGC1414 | 691067II [symbol] | 0.461 | 0.428 | −0.667 | −0.002 | 0.002 | 0.903 | −0.063 | −1.288 | −0.39 | −0.583 | 0.448 | −0.124 | −1.103 | 0.237 | −0.239 |
| MGC1428 [symbol] | 695016II [symbol] | −0.376 | 0.543 | −0.506 | 0.213 | 0.13 | −0.072 | −0.864 | −0.049 | 0.06 | −0.23 | 0.034 | −0.791 | −0.649 | 0.118 | −0.491 |
| MGC148 [symbol] | 693346II [symbol] | −0.0925 | 1.297 | −0.2385 | −0.4665 | 1.045 | −1.06 | −1.22 | 2.852 | −0.21 | −0.6625 | −0.9305 | 1.525 | 1.255 | 1.04 | −0.2855 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| MGC1560 [symbol] | 681291II [symbol] | −0.176 | −0.139 | −1.006 | 1.169 | 0.351 | −0.167 | −0.427 | 0.246 | 0.11 | −0.673 | 0.461 | −0.206 | −0.557 | 0.372 | 1.806 |
| MGC1663 [symbol] | 695059II [symbol] | −2.565 | −0.2805 | −0.4805 | 3.131 | 5.665 | −0.2125 | −2.567 | −2.923 | −0.17 | −4.364 | −1.289 | −2.587 | 0.4435 | −2.169 | −0.6285 |
| MGC1673 [symbol] | 684966II [symbol] | −0.0425 | 0.5016 | 0.4745 | −0.1335 | −0.1705 | 0.4975 | −0.6335 | 0.1375 | −0.13 | −0.6055 | 0.0065 | 0.3095 | 2.728 | −0.4235 | −0.0715 |
| MGC2053 [symbol] | 692614II [symbol] | −0.242 | −0.953 | −0.581 | −0.768 | −1.16 | −0.458 | 0.366 | −0.606 | −0.38 | 0.033 | −1.137 | −0.505 | −1.082 | −1.38 | −0.1226 |
| MGC2398 [symbol] | 684508II [symbol] | 1.494 | 0.0478 | 0.043 | −0.622 | −0.244 | −0.452 | −0.385 | −0.228 | −0.45 | 0.486 | −0.082 | −0.304 | −0.446 | 0.377 | 1.55 |
| MGC2610 | 685994II [symbol] | −1.609 | 0.0145 | 1.463 | 0.7695 | −0.0205 | −0.9605 | −0.6675 | −0.4385 | 0.62 | −0.4885 | 1.139 | −2.041 | −1.283 | 0.9666 | 0.6895 |
| MGC2714 | 681884II [symbol] | 0.1745 | −0.4625 | 1.662 | −0.4645 | −0.3295 | −0.4165 | 0.2175 | 1.032 | −0.15 | 0.5315 | −0.6355 | 0.0355 | −0.2135 | 0.6655 | 0.6125 |
| MGC2716 [symbol] | 682082II [symbol] | 3.139 | −0.168 | −0.936 | −0.026 | 0.246 | −0.71 | −0.231 | −0.925 | −0.69 | −0.591 | −0.823 | 0.518 | 1.363 | 0.404 | 3.06 |
| MGC2716 [symbol] | 685085II [symbol] | 0.144 | 1.937 | −1.006 | −0.89 | −0.182 | −1.427 | 1.079 | 0.298 | −0.88 | −0.45 | −0.736 | 0.418 | −0.696 | −0.565 | −0.329 |
| MGC2716 [symbol] | 687819II [symbol] | 3.754 | 0.513 | −0.616 | −0.005 | 1.548 | −0.831 | 0.436 | −0.547 | −0.89 | −0.587 | −0.101 | 1.493 | 1.65 | 0.919 | 3.406 |
| MGC2716 [symbol] | 694146II [symbol] | 4.665 | 1.541 | 0.384 | 0.968 | 0.989 | 0.371 | 1.019 | −0.029 | −1.26 | 0.541 | 1.021 | 2.288 | 1.752 | 1.337 | 4.368 |
| MGC2716 [symbol] | 695966II [symbol] | 4.636 | 1.191 | 0.391 | 0.954 | 2.265 | 0.217 | 1.424 | 0.096 | −0.65 | 0.438 | 1.062 | 2.447 | 2.65 | 2.274 | 4.197 |
| MGC2987 [symbol] | 692023II [symbol] | −0.5777 | 0.2763 | 0.8963 | 0.2153 | −0.0718 | −0.7687 | 0.6853 | 1.386 | 0.13 | 0.4183 | −0.4407 | 0.8153 | −0.1907 | 0.1963 | 1.155 |
| MGC3212 [symbol] | 693269II [symbol] | −0.766 | 0.368 | −1.473 | 1.696 | 1.778 | 1.504 | −1.208 | −1.198 | 0.68 | −0.784 | 0.506 | −1.072 | 0.113 | −0.39 | −1.277 |
| MGC3321 [symbol] | 692025II [symbol] | −0.419 | −0.744 | 1.469 | 0.061 | 0.251 | −0.152 | 1.263 | −0.012 | −0.18 | −0.462 | −0.901 | 1.387 | 0.69 | −0.394 | 0.608 |
| MGC3464 [symbol] | 692812II [symbol] | 1.027 | 0.032 | 3.023 | 1.116 | 1.256 | 0.312 | 2.957 | 3.742 | 0.8 | 0.233 | 0.901 | 3.43 | 0.343 | 0.588 | 3.246 |
| MGC3492 [symbol] | 691559II [symbol] | 0.489 | 0.152 | 0.016 | −0.812 | −0.022 | −0.144 | 0.465 | 3.041 | −0.55 | 1.358 | −0.867 | 0.343 | 0.851 | 0.468 | 2.074 |
| MGC4021 [symbol] | 695104II [symbol] | 0.13 | 1.06 | 0.1 | 4.29 | 1.276 | 1.432 | 0.104 | 0.293 | 0.18 | 0.167 | 2.875 | −0.145 | 0.115 | 0.003 | 0.078 |
| MGC4251 | 693489II [symbol] | −0.437 | 0.275 | −2.304 | −0.452 | 0.88 | 2.443 | −0.629 | 0.301 | 0.84 | 0.821 | 1.135 | 0.082 | −1.457 | −0.508 | −0.24 |
| MGC4308 | 691305II [symbol] | 0.23 | −1.117 | 0.507 | −0.28 | −0.713 | −0.666 | 0.181 | 1.198 | −0.2 | 0.758 | −0.676 | 0.067 | 0.271 | 0.355 | 1.804 |
| MGC4606 | 683786II [symbol] | −0.5625 | −0.3565 | −0.1605 | 0.4475 | −0.1555 | 1.021 | 0.1165 | −1.009 | 0.62 | −0.4405 | −0.4785 | 0.4225 | 0.7905 | −0.7345 | −0.5155 |
| MGC4659 | 684418II [symbol] | −1.444 | −1.19 | −1.232 | 0.4725 | 2.784 | 1.181 | −0.8415 | −0.4675 | 0.67 | −2.245 | −0.2445 | −0.3495 | −1.187 | −1.624 | 0.3125 |
| MIA | 695303II [symbol] | −2.46 | −1.462 | 2.38 | −0.9 | −1.681 | −1.609 | 3.33 | 2.3 | 3.58 | −2.55 | −1.082 | 3.59 | −2.261 | −1.223 | 2.33 |
| MID1 | 687747II [symbol] | −0.7775 | −0.1055 | 0.9205 | 0.2315 | 0.3785 | 0.0085 | −0.1855 | 0.0805 | −0.13 | 0.1755 | 0.4105 | −1.613 | −0.4025 | 0.3885 | 0.2065 |
| MID1 | 690115II [symbol] | −0.621 | −0.727 | 2.285 | −0.664 | 0.628 | −1.322 | 1.897 | 0.702 | 0.08 | 0.889 | −0.941 | 0.421 | −0.961 | 0.047 | 1.783 |
| MID1 | 690599II [symbol] | −0.407 | −2.348 | 2.853 | −0.593 | 0.231 | −1.109 | 2.489 | 0.945 | 0.29 | 0.938 | −0.969 | 0.502 | −0.735 | 0.609 | 2.927 |
| MLF1IP | 684863II [symbol] | 0.2942 | −0.5558 | −1.644 | 0.5222 | −0.0458 | −1.527 | −0.4268 | 0.0993 | −0.57 | −1.451 | −0.3018 | −1.536 | 0.0213 | 0.3902 | −0.6048 |
| MLSTD1 | 689505II [symbol] | 1.069 | 0.4625 | −0.5125 | −0.0775 | 0.4505 | 0.7135 | 1.169 | −0.4885 | −0.22 | 1.889 | −0.7355 | −0.1425 | −0.5785 | −0.7215 | 1.242 |
| MMP1 | 697289II [symbol] | 3.534 | −2.645 | −3.241 | −2.835 | −2.742 | −2.644 | −2.856 | 0.529 | −0.15 | 0.386 | −2.056 | −2.868 | 0.178 | −0.816 | −1.472 |
| MMP7 | 697274II [symbol] | 2.484 | 4.54 | 4.881 | 2.325 | 3.641 | 2.443 | 3.423 | 4.807 | 0.13 | 0.883 | 3.62 | 4.256 | 1.54 | 4.177 | 5.149 |
| MNAT1 | 687038II [symbol] | −0.712 | −0.26 | −1.864 | 0.387 | −1.08 | 2.46 | −1.389 | −1.684 | 0.09 | −1.579 | 0.09 | −1.57 | −1.15 | −0.96 | −2.07 |
| MRPL14 | 686347II [symbol] | 0.082 | −1.09 | 0.493 | 0.336 | −0.024 | 0.238 | 0.024 | 0.188 | −0.33 | 0.262 | −0.733 | 0.206 | 0.523 | 0.233 | 0.075 |
| MRPL15 | 693549II [symbol] | −0.728 | 0.645 | 0.237 | −1.357 | −1.071 | −0.052 | 0.999 | −0.45 | −0.28 | 0.172 | −0.511 | 0.114 | −0.988 | −0.599 | −0.276 |
| MRPL27 | 697164II [symbol] | −0.322 | 1.401 | −1.101 | −0.193 | 0.505 | −0.102 | −0.677 | −0.297 | −0.03 | 0.431 | 0.054 | −0.105 | −0.265 | −0.876 | −0.532 |
| MRPL34 | 688498II [symbol] | 0.226 | −0.44 | 0.711 | 0.771 | 0.599 | 0.026 | −0.268 | −0.116 | −0.27 | 0.237 | 0.132 | −0.935 | −0.23 | −0.309 | 0.246 |
| MRPL43 | 683308II [symbol] | −0.9225 | −0.6655 | −1.566 | −0.7975 | 0.1285 | −0.8445 | 0.7545 | −0.5325 | 0.08 | −0.6155 | 0.6905 | −0.2035 | −0.1895 | 0.1845 | 0.8545 |
| MRPL45 | 687058II [symbol] | 0.4945 | 0.0165 | 0.0655 | −0.2825 | −0.4955 | 0.4785 | −0.4605 | 0.4495 | 0.06 | 0.2515 | 0.0945 | −0.2195 | −0.7085 | 1.785 | 0.1535 |
| MRPL48 | 683204II [symbol] | 0.0865 | 0.6395 | 0.2105 | −0.2095 | 0.2165 | −0.4535 | −0.0065 | 0.5745 | −0.23 | 0.8515 | 0.2375 | −0.0095 | −0.2005 | −0.2955 | 0.3475 |
| MRPL9 | 682558II [symbol] | −0.4365 | 0.2855 | 0.9755 | −0.0905 | −0.1825 | −0.4285 | 0.4865 | 0.5575 | 0.17 | 0.4455 | −0.2975 | 0.4165 | −0.0115 | −0.1735 | 0.5565 |
| MRPS14 | 691200II [symbol] | 0.0965 | −0.3455 | 0.4885 | 0.0265 | 0.4645 | −0.4155 | 0.1725 | 0.0385 | −0.25 | 0.0495 | −0.0665 | −0.6265 | −0.1295 | 0.2605 | −0.3345 |
| MRPS15 | 686445II [symbol] | −0.108 | 0.312 | −0.109 | 0.033 | 0.977 | 0.385 | 0.642 | 0.11 | −0.23 | 0.616 | 0.046 | 0.241 | 0.106 | −0.683 | −0.096 |
| MRPS17 | 695189II [symbol] | 3.623 | 1.264 | 0.404 | 2.508 | 2.509 | 1.48 | 3.341 | 0.301 | −0.47 | 0.408 | 2.127 | 1.882 | 2.415 | 1.738 | 3.544 |
| MRPS24 | 681503II [symbol] | −0.088 | 0.713 | −0.105 | 0.044 | 0.628 | −0.358 | 0.054 | 0.413 | −0.27 | 0.75 | −0.387 | 0.08 | −0.459 | −0.599 | 0.43 |
| MRPS25 | 695169II [symbol] | −0.619 | −0.441 | 0.298 | −1.312 | −0.424 | −0.889 | −0.768 | −1.157 | −0.3 | −0.438 | −0.995 | −0.889 | −1.242 | −0.728 | 0.314 |
| MRPS31 | 689671II [symbol] | 0.144 | −0.128 | −0.253 | −0.225 | 0.03 | −0.652 | −0.01 | −0.545 | 0.33 | 0.221 | −0.23 | −0.836 | −0.075 | −0.1831 | 0.947 |
| MRPS6 | 692172II [symbol] | −0.331 | −0.002 | −0.242 | −1.433 | −0.626 | −0.823 | −0.224 | −0.042 | 0.08 | 1.148 | −0.409 | −0.109 | −0.185 | −0.607 | −0.011 |
| MS4A1 | 690488II [symbol] | 0.052 | 0.383 | −0.182 | −0.256 | 0.741 | −0.075 | 1.492 | −0.082 | −0.17 | −0.183 | 0.025 | 0.285 | −0.251 | −0.068 | 2.809 |
| MSH2 | 686029II [symbol] | −0.501 | 0.221 | 1.121 | 0.01 | −0.187 | −1.077 | 0.501 | 1.211 | −0.08 | 0.899 | −0.219 | 0.026 | 0.729 | −0.148 | 0.486 |
| MSH3 | 692736II [symbol] | 0.021 | −0.538 | −0.022 | 0.435 | −0.025 | −0.205 | −0.913 | −0.246 | −0.3 | 0.002 | −0.627 | −1.404 | 0.092 | −0.357 | −0.909 |
| MSMB | 695442II [symbol] | −0.017 | 0.094 | −2.049 | 0.671 | −0.719 | −0.652 | −1.294 | −0.296 | 0.72 | −1.287 | −0.754 | −0.789 | 0.288 | 0.062 | −1.598 |
| MTA1 | 690961II [symbol] | 0.3835 | −0.215 | 0.0465 | 0.1515 | 0.1675 | −0.7195 | −0.3695 | 0.2205 | 0.28 | −1.244 | −0.3545 | −0.4705 | −0.2455 | −0.1235 | −0.8665 |
| MTA3 | 684982II [symbol] | 0.305 | 0.438 | −0.209 | 0.11 | −0.053 | 0.183 | −0.265 | −0.405 | 0.24 | 0.655 | 0.511 | 0.231 | 0.037 | 0.021 | −1.018 |
| MTAC2D1 | 689442II [symbol] | 0.728 | 0.131 | −0.29 | 0.047 | −1.593 | −0.762 | −0.649 | −0.237 | 0.35 | −1.61 | −0.007 | −0.491 | 0.238 | 0.26 | −0.039 |
| MTCH1 | 694039II [symbol] | −0.118 | −0.436 | −0.22 | −0.73 | −0.087 | −0.216 | 0.159 | −0.317 | 0 | −0.289 | −0.071 | −0.319 | −0.493 | −0.36 | 0.318 |
| MTHFD1 | 693532II [symbol] | −1.128 | −1.275 | −1.143 | −1.241 | −1.363 | −0.81 | −0.945 | −0.755 | −0.33 | 0.625 | −0.877 | −1.612 | −0.228 | −1.336 | −0.993 |
| MTMR2 | 683517II [symbol] | −0.615 | 0.477 | −0.922 | −0.447 | 0.354 | −0.059 | −0.099 | −0.292 | −0.32 | −1.202 | 0.598 | 0.665 | −0.714 | −0.399 | −0.166 |
| MUC1 | 691634II [symbol] | 0.0405 | −0.7375 | −4.868 | 0.5765 | 0.3285 | −0.8855 | −0.9815 | 0.3255 | 0.11 | −3.353 | 0.4676 | −1.997 | −1.835 | 1.043 | −1.332 |
| MUC20 | 691484II [symbol] | 1.173 | 1.133 | −0.682 | −0.482 | −0.116 | 0.785 | −0.553 | 1.795 | 0.31 | −1.423 | −0.213 | 1.244 | −0.993 | 0.38 | −0.152 |
| MUM1 | 684755II [symbol] | 0.209 | 0.272 | −0.578 | −0.208 | −0.047 | 0.65 | −0.044 | −1.073 | 0.46 | −0.778 | 0.174 | 0.062 | 0.067 | −0.035 | −0.441 |
| MXI1 | 692501II [symbol] | 0.047 | −0.081 | −0.101 | 0.072 | 0.1359 | 0.637 | −0.943 | 0.185 | 0.51 | −1.507 | −0.225 | 0.295 | −1.305 | 0.605 | −0.035 |
| MYB | 693882II [symbol] | 0.284 | 0.573 | −0.147 | 1.194 | 0.872 | 1.755 | 0.083 | −1.123 | 2.42 | −0.374 | 1.767 | 0.313 | 1.034 | −0.442 | −0.292 |
| MYBL1 | 685207II [symbol] | −0.615 | 0.999 | 1.355 | −0.615 | −1.87 | 1.932 | 3.375 | 3.705 | −0.8 | −0.855 | 1.366 | 1.774 | −0.663 | −1.213 | 2.455 |
| MYBL2 | 683074II [symbol] | 0.7265 | 0.2065 | 0.8905 | −0.8605 | −0.9635 | 0.0085 | 1.855 | 0.6225 | −0.93 | 2.306 | −0.6395 | 0.9765 | 0.0615 | −0.0085 | 1.161 |
| MYO10 | 682215II [symbol] | 0.8285 | 0.1595 | 1.503 | 0.1475 | −0.7415 | 0.0045 | −1.05 | 1.968 | −0.14 | 0.2535 | −1.046 | 2.171 | −0.0795 | −0.2775 | 0.9265 |
| MYO6 | 695860II [symbol] | 2.153 | −0.0435 | 0.4275 | −0.0585 | 0.5975 | 0.2565 | 0.3285 | −0.2276 | 0.6 | 1.84 | −0.3376 | 0.1645 | 0.0395 | −0.7495 | 1.678 |
| MYST3 | 772896II [symbol] | 0.628 | 2.009 | 0.82 | 0.654 | 1.024 | 1.64 | −0.039 | 0.28 | 0.2 | 0.107 | 0.847 | 2.472 | 0.28 | −0.158 | 0.444 |
| NALP2 | 692203II [symbol] | −0.809 | −1.618 | 2.208 | 0.452 | −1.601 | 1.239 | −1.165 | 2.478 | 0.54 | 3.398 | −1.619 | 1.81 | 0.076 | −1.468 | −0.978 |
| NALP6 | 684014II [symbol] | 0.191 | −0.375 | −0.25 | 0.772 | 0.142 | 2.628 | 0.07 | −0.146 | −0.24 | −0.186 | −0.409 | −0.302 | −0.014 | 0.062 | −0.127 |
| NANS | 697330II [symbol] | 0.716 | 0.168 | −1.101 | −0.396 | 0.32 | −0.714 | −0.246 | 0.463 | −0.2 | −0.481 | 0.025 | −0.986 | −0.768 | −0.144 | 0.503 |
| NARF | 687421II [symbol] | 2.114 | 0.2955 | −1.331 | 0.7465 | 0.2785 | −0.0375 | 0.0325 | −0.7565 | −0.19 | −0.5035 | −0.0515 | 0.5775 | 0.3595 | 0.6255 | −0.0775 |
| NAT2 | 697768II [symbol] | −0.681 | −1.005 | −1 | −0.586 | −0.655 | −0.144 | −1.146 | 0.303 | 0.84 | −0.448 | 0.939 | 0.43 | −0.413 | −0.544 | −0.355 |
| NAV2 | 685130II [symbol] | 0.1155 | −1.64 | 0.7815 | 0.5675 | −1.239 | −0.5265 | −0.0835 | −0.3445 | 1.19 | −0.7775 | −0.8036 | −0.8335 | 0.0095 | 1.186 | 1.884 |
| NDE1 | 690898II [symbol] | −0.6365 | 0.8225 | −1.057 | 0.2515 | 0.5865 | 1.662 | −0.0855 | −0.2635 | 0.19 | −1.159 | 0.2555 | −0.2755 | −0.6445 | −0.4655 | −0.1555 |
| NDP52 | 684577II [symbol] | −0.5285 | 1.247 | −1.306 | 0.3435 | 1.171 | −0.4875 | −0.7915 | 0.6525 | −0.03 | −0.8945 | 0.8466 | −0.3265 | −0.6715 | 0.5185 | 0.3595 |
| NDRG2 | 686705II [symbol] | −1.044 | 0.311 | −0.665 | 0.545 | −0.849 | 0.088 | 0.157 | 1.588 | 0.69 | −1.486 | −0.76 | 1.877 | −1.45 | −0.692 | −0.246 |
| NDUFA7 | 682916II [symbol] | −0.215 | −0.318 | 0.508 | 1.222 | 1.054 | 0.372 | 0.005 | −0.553 | −0.42 | −0.808 | 0.669 | 0.004 | −0.183 | −0.427 | −0.511 |
| NDUFB1 | 695310II [symbol] | 0.026 | −0.633 | 0.041 | 0.619 | −0.255 | −0.246 | −0.531 | −1.221 | −0.59 | −0.829 | −0.377 | −0.952 | 0.188 | −0.425 | −0.49 |
| NDUFB10 | 681833II [symbol] | 0.613 | 0.579 | −0.362 | −0.094 | 1.028 | 1.16 | −0.226 | −0.607 | −0.04 | −0.056 | −0.121 | 0.203 | 0.078 | −0.48 | −0.147 |
| NDUFB2 | 688062II [symbol] | −0.2695 | −0.3335 | 0.1945 | 0.5455 | 0.5295 | −0.1705 | 0.0625 | 0.2325 | −0.21 | −0.1055 | 0.2625 | −0.5905 | 0.2505 | −0.4865 | −0.7575 |
| NDUFB5 | 686352II [symbol] | −0.5635 | 0.5835 | 0.0025 | −0.0025 | −0.2945 | −0.2985 | 0.0955 | 1.047 | −0.15 | 0.0705 | 0.3775 | −0.5975 | −0.9385 | 0.5485 | 1.659 |
| NDUFB6 | 686626II [symbol] | −0.5065 | −0.6165 | 0.1776 | 0.3425 | −0.2975 | 0.0455 | −0.0145 | 0.0145 | −0.17 | 0.4155 | 0.2485 | −1.272 | 0.1065 | −0.1775 | 0.2195 |
| NDUFC2 | 696213II [symbol] | −0.265 | 0.207 | −0.428 | −0.368 | −0.967 | 0.238 | −0.826 | −0.164 | −0.17 | −0.613 | 0.37 | −0.873 | 2.711 | −0.571 | 0.089 |
| NEBL | 690781II [symbol] | 0.6305 | 0.7785 | 1.161 | 0.0815 | 0.2535 | 0.8845 | −0.6985 | 1.323 | −0.27 | −0.5495 | −0.4895 | −0.3935 | 0.8795 | −0.4026 | 0.7955 |
| NEK2 | 695375II [symbol] | −0.865 | −0.997 | −0.202 | −1.682 | −2.323 | −2.542 | 0.416 | 0.865 | −1.24 | −0.644 | −2.759 | −0.385 | −0.642 | −1.383 | 0.264 |
| NEK6 | 695042II [symbol] | 0.616 | 0.502 | 0.478 | −0.718 | −0.036 | −0.912 | 1.532 | 1.132 | −0.01 | 0.256 | −0.438 | 1.573 | 0.046 | −0.396 | −0.512 |
| NELL2 | 688574II [symbol] | 0.125 | −0.245 | −0.997 | 2.327 | −0.572 | 3.003 | 0.169 | −1.275 | −0.04 | −1.221 | −0.549 | −0.545 | −0.509 | −1.014 | 0.092 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| NEURL | 681900II [symbol] | −0.5415 | 0.7855 | −1.24 | 0.0145 | 0.6495 | 0.7015 | −1.268 | 0.1035 | 0.3 | −0.2775 | 1.094 | 0.2315 | −0.6605 | −0.0885 | −0.6035 |
| NFE2L3 | 683131II [symbol] | 0.3585 | 0.4925 | 0.9985 | 0.1285 | 0.5645 | 0.9705 | 1.409 | 2.269 | −0.54 | 2.889 | 0.0635 | 1.632 | 1.903 | −0.6415 | 2.589 |
| NFIA | 692198II [symbol] | 1.49 | 1.862 | 1.678 | 2.212 | 2.063 | 1.774 | 0.725 | 1.603 | 1.03 | −1.425 | 1.74 | 3.167 | −1.289 | 2.991 | 1.376 |
| NFIB | 681529II [symbol] | −0.825 | 0.549 | −0.533 | −0.494 | 0.945 | 4.215 | −0.285 | −0.081 | 0.44 | 0 | 3.948 | −0.658 | −0.676 | −0.2604 | 0.832 |
| NINJ1 | 684190II [symbol] | −0.0645 | 0.4955 | −1.087 | 0.5255 | 1.11 | 0.9215 | −1.137 | 0.0355 | 0.31 | −0.5005 | 0.7015 | −0.3425 | −0.2505 | 0.1015 | −0.1645 |
| NIPSNAP [symbol] | 697441II [symbol] | −0.149 | −0.137 | 0.179 | 0.213 | 0.114 | −0.389 | 0.748 | 0.329 | 0 | −0.687 | 0.826 | −1.067 | −0.507 | −0.308 | 1.914 |
| NKX6-2 | 682635II [symbol] | 0.093 | 0.262 | −0.039 | −0.039 | 0.408 | −0.176 | −0.143 | 0.014 | −0.37 | 0.414 | −0.106 | 0.211 | −0.003 | −0.069 | −0.08 |
| NME1 | 689970II [symbol] | 0.0565 | 0.4475 | 0.3605 | −0.1005 | −0.0595 | −0.3415 | −0.0135 | 0.4725 | −0.06 | 0.4765 | −0.7645 | 0.0715 | 0.3835 | −0.1995 | 0.6215 |
| NME2 | 681665II [symbol] | −0.127 | 1.052 | 0.488 | −0.068 | 0.372 | −0.307 | −0.037 | 0.709 | −0.1 | 0.327 | −0.321 | 0.086 | −0.013 | −0.372 | 0.62 |
| NME3 | 682225II [symbol] | 0.2685 | 0.3955 | −0.2965 | 0.3925 | 0.8915 | 1.168 | −0.9755 | −1.248 | 0.32 | −1.598 | 0.6865 | 0.0155 | −0.0155 | −0.2195 | −1.323 |
| NMES1 | 689620II [symbol] | 0.634 | 0.602 | 1.552 | 0.612 | 2.4 | 1.376 | −0.614 | 1.404 | 0.35 | −1.112 | 1.088 | −0.479 | 0.039 | −0.632 | 0.751 |
| NMI | 690834II [symbol] | −0.035 | −1.638 | 1.095 | −1.155 | −0.956 | −0.561 | 1.175 | 2.125 | −0.09 | 0.265 | 0.934 | −0.288 | 0.131 | 0.528 | 1.965 |
| NOC4 | 691497II [symbol] | −0.1545 | 0.6495 | 0.2775 | −0.4865 | 0.4255 | −0.4205 | 0.9455 | 1.625 | −0.14 | 0.6175 | −0.4985 | 0.5915 | 0.2725 | −0.3785 | 0.8915 |
| NOLC1 | 689311II [symbol] | −0.103 | −0.253 | 0.16 | −0.507 | 0.249 | 0.321 | 0.439 | −0.044 | 0.34 | 0.456 | 0.024 | −0.024 | 0.159 | 0.382 | 0.607 |
| NotFound [symbol] | 681142II | −0.526 | 0.229 | 2.073 | −1.117 | 0.782 | −0.54 | 1.731 | 0.652 | −0.51 | 0.713 | −0.443 | 0.441 | −0.767 | 0.12 | 1.692 |
| NotFound [symbol] | 681155II | 0.0735 | −0.2176 | 1.615 | −0.1015 | −0.2445 | −0.0875 | 0.1865 | 1.462 | −0.04 | 0.7145 | −0.0105 | −0.2405 | −0.5405 | 0.2425 | 0.4405 |
| NotFound [symbol] | 681349II | 0.1415 | 0.6825 | −0.4085 | 0.1215 | −0.0275 | 0.2535 | −0.4945 | −0.5435 | 0.3 | 0.3485 | 0.0705 | −0.5125 | −0.5705 | 0.3905 | −0.0625 |
| NotFound [symbol] | 681357II | 1.988 | 0.0535 | −0.0225 | −0.2815 | 0.7265 | −0.1295 | 0.0345 | −0.5785 | −0.37 | −0.3715 | 0.1135 | 0.7535 | −0.0025 | 0.0545 | 1.524 |
| NotFound [symbol] | 681576II | 0.7615 | −1.466 | 0.6355 | 1.858 | 0.1575 | −0.1875 | −2.32 | −1.13 | 0.46 | −0.6235 | −0.2045 | −0.3825 | 0.3755 | 0.9575 | −0.0395 |
| NotFound [symbol] | 681606II | −0.05 | 0.429 | 0.696 | 0.292 | 1.552 | −0.72 | 0.576 | 0.333 | −0.61 | 0.973 | −1.043 | 0.226 | −0.034 | 0.034 | −0.196 |
| NotFound [symbol] | 681958II | 0.7615 | −1.878 | 0.5735 | −1.158 | −0.0495 | −1.297 | 0.4395 | −0.3605 | −0.89 | −1.957 | 0.7385 | −0.6495 | 0.4055 | 0.1285 | 2.757 |
| NotFound [symbol] | 682219II | −0.078 | 0.511 | 1.057 | 0.222 | 0.157 | 0.509 | 0.337 | −0.099 | 2.41 | 1.003 | 1.812 | 0.229 | 0.44 | −0.206 | −0.208 |
| NotFound [symbol] | 682298II | −0.142 | 0.984 | 0.675 | −0.489 | 0.053 | −0.347 | 1.037 | 0.611 | 0.06 | 0.987 | −0.301 | 0.401 | 0.125 | −0.74 | 1.297 |
| NotFound [symbol] | 682699II | 1.928 | 4.213 | −1.904 | 1.864 | 2.233 | 0.83 | −2.294 | −2.196 | −0.09 | −1.575 | 3.59 | −1.883 | 2.401 | 2.276 | −3.381 |
| NotFound [symbol] | 683354II | 0.638 | 0.459 | 2.537 | 0.069 | 0.525 | 0.563 | 2.018 | 1.762 | 0.26 | 0.138 | 0.659 | 1.794 | 0.772 | 0.6462 | 1.823 |
| NotFound [symbol] | 683540II | −1.183 | −0.4755 | 0.5875 | 0.5405 | −0.1605 | −0.4175 | −0.8615 | 1.023 | −0.72 | 0.2505 | −0.6415 | 0.8745 | −1.246 | −1.315 | −0.6415 |
| NotFound [symbol] | 683552II | 0.0055 | 0.2235 | 0.4335 | −0.2205 | −0.1505 | 0.1025 | 0.5325 | −0.1895 | −0.38 | 0.5275 | −0.4385 | 0.1865 | −0.8285 | −0.3025 | 0.4825 |
| NotFound [symbol] | 683669II | 0.025 | 0.175 | −0.132 | −0.18 | 0.066 | 0.035 | 0.024 | 0.588 | −0.29 | −0.221 | 0.105 | 1.786 | −0.316 | −0.084 | −0.217 |
| NotFound [symbol] | 684000II | −0.83 | 0.83 | −0.095 | −0.423 | −1.895 | 0.299 | −1.428 | −0.361 | 1.33 | −2.444 | 1.105 | 0.67 | −0.705 | −3.003 | −1.221 |
| NotFound [symbol] | 684333II | 1.278 | 0.22 | 2.931 | 0.426 | 0.05 | 0.3 | 3.363 | 2.251 | 0.01 | 3.168 | 0.51 | 1.7 | 0.03 | 0.6 | 2.268 |
| NotFound [symbol] | 684481II | 0.7775 | 2.64 | −1.854 | −0.1145 | 0.4885 | 0.5585 | −1.871 | −1.98 | 0.08 | 1.012 | 0.3615 | −1.426 | −0.0225 | 0.0975 | −2.095 |
| NotFound [symbol] | 685169II | 0.554 | 0.167 | −1.452 | 0.608 | −0.219 | −0.429 | −0.873 | −1.963 | 0.98 | −0.406 | 0.1 | −1.53 | 0.9 | 0.018 | −2.031 |
| NotFound [symbol] | 685265II | 0.6765 | −0.0095 | −0.0775 | 0.4855 | −0.1345 | 0.6035 | −0.1465 | 0.3905 | 1.01 | −1.509 | 0.4215 | −0.4325 | 0.2035 | 1.557 | 0.0515 |
| NotFound [symbol] | 685572II | −0.17 | −0.921 | 1.159 | −0.252 | 0.638 | −0.876 | 0.131 | 0.308 | 0 | 0.126 | −0.648 | −1.059 | 0.014 | 0.593 | 0.095 |
| NotFound [symbol] | 665625II | −0.6315 | 0.7585 | −0.7915 | 0.1555 | −0.0365 | 0.3865 | −0.4325 | 0.3775 | −0.15 | 1.107 | 0.2475 | 1.653 | −0.1185 | 0.3165 | −0.2345 |
| NotFound [symbol] | 685756II | −0.768 | −2.088 | −0.842 | 1.596 | 1.164 | 1.391 | −1.655 | 0.289 | 1.22 | −1.251 | 2.163 | −1.392 | 2.009 | 0.058 | −1.748 |
| NotFound [symbol] | 685823II | −0.302 | 0.015 | −0.493 | 0.049 | 0.154 | 0.116 | −0.747 | −0.964 | 0.31 | −0.403 | −0.29 | −0.677 | −0.69 | −0.527 | −1.131 |
| NotFound [symbol] | 686027II | 0.288 | −0.356 | 0.473 | −0.57 | 0.087 | −0.845 | 0.403 | 2.27 | −0.06 | −0.568 | −0.03 | 0.916 | −0.443 | −0.475 | 1.329 |
| NotFound [symbol] | 688055II | −0.203 | 1.565 | −1.482 | 4.633 | 2.137 | 2.267 | −0.51 | −0.772 | 2.99 | −0.73 | 4.462 | −0.787 | −0.367 | −0.927 | −0.403 |
| NotFound [symbol] | 686477II | 0.424 | −0.119 | 0.719 | 0.039 | 0.607 | −1.296 | −0.668 | 1.055 | 0.02 | −0.391 | −0.673 | 0.021 | 0.265 | −0.021 | −0.428 |
| NotFound [symbol] | 686759II | −0.244 | 0.01 | 0.668 | 0.42 | 1.075 | 0.197 | −0.019 | −0.443 | −0.37 | −0.823 | 1.177 | −0.439 | −0.178 | 0.445 | 0.732 |
| NotFound [symbol] | 686948II | −0.076 | 0.307 | 2.111 | −0.205 | 0.026 | 0.048 | 0.495 | 1.288 | 0.25 | 1.112 | −0.156 | 0.701 | 0.124 | 0.206 | 0.261 |
| NotFound [symbol] | 687368II | 0.4355 | 0.0065 | 0.1335 | −0.2915 | 0.4215 | −0.8675 | −0.5485 | 1.17 | −0.23 | −0.2995 | −0.6575 | 0.0075 | −0.1225 | −0.0065 | −0.5745 |
| NotFound [symbol] | 687761II | 0.2845 | 0.5925 | 0.0145 | 0.3225 | 0.1195 | 0.5795 | 0.3535 | 0.0085 | −0.92 | −0.2535 | 0.0965 | −0.0085 | −0.3355 | −0.4585 | −0.8105 |
| NotFound [symbol] | 688033II | −0.6635 | 0.2795 | 0.0715 | 0.3885 | 0.3575 | −0.4715 | −0.8135 | −0.3855 | 0.28 | −0.1965 | 0.1125 | −1.639 | 0.0475 | 0.0095 | −0.5815 |
| NotFound [symbol] | 688163II | 3.267 | 0.1885 | −0.7616 | −0.3065 | 0.2265 | −0.4125 | 0.3855 | −0.5145 | −0.69 | −0.7435 | −0.3895 | 0.5145 | −0.0555 | −0.1016 | 3.209 |
| NotFound [symbol] | 688264II | −0.6395 | 1.879 | −0.3455 | 0.1705 | 0.8475 | −0.6455 | −1.138 | 1.448 | 0.09 | −1.301 | −1.682 | −0.1705 | −0.3755 | −0.2616 | −0.2945 |
| NotFound [symbol] | 688843II | 0.5615 | 1.331 | −0.9755 | 0.0415 | 2.033 | −0.7745 | −0.2605 | 0.6025 | −0.39 | −0.4495 | −0.9205 | 0.4115 | −0.6685 | −0.6135 | −1.184 |
| NotFound [symbol] | 688964II | 0.0875 | 0.1165 | 0.4305 | −0.1705 | 0.6775 | −1.471 | −0.1875 | 1.457 | −0.2 | 0.0745 | −0.7075 | −0.1325 | 0.1565 | 0.1675 | −0.2955 |
| NotFound [symbol] | 688988II | −0.063 | −0.574 | 0.414 | 0.728 | 1.193 | 1.076 | −0.676 | −1.129 | 0.01 | −2.251 | 0.717 | 0.86 | 0.809 | −0.873 | 1.334 |
| NotFound [symbol] | 689368II | −0.3785 | 0.9765 | 0.4875 | −0.4975 | 0.9045 | −0.4545 | −0.6945 | 0.8005 | −0.35 | −0.6055 | −0.0135 | 0.5035 | −0.0875 | −0.2945 | −1.217 |
| NotFound [symbol] | 689732II | −1.325 | 0.421 | 0.478 | 0.894 | 1.105 | −1.631 | −1.564 | 1.447 | 0.79 | −1.805 | 0.337 | −0.045 | −1.142 | −0.221 | −1.073 |
| NotFound [symbol] | 690073II | 0.0915 | −0.9085 | −0.7165 | −0.9815 | 0.4615 | 0.0455 | 0.4775 | 0.1385 | −0.44 | 0.0115 | −0.5555 | −0.2435 | 1.679 | 0.0245 | 0.2845 |
| NotFound [symbol] | 690533II | −0.5435 | 1.28 | −0.1285 | −0.5505 | 1.077 | −0.9176 | −0.6145 | 1.137 | −0.18 | −1.907 | −1.152 | 0.1605 | 0.2255 | −1.363 | −1.575 |
| NotFound [symbol] | 690571II | −0.403 | −0.009 | −0.43 | −0.172 | 0.171 | 0.107 | −0.082 | 0.26 | −0.43 | −0.269 | 0.531 | 0.608 | −0.62 | −0.241 | 0.235 |
| NotFound [symbol] | 690683II | 0.291 | 0.115 | 1.688 | 0.59 | −0.008 | 0.149 | −0.182 | −0.132 | 0.26 | −0.414 | −0.414 | 0.189 | 0.309 | −0.21 | −0.348 |
| NotFound [symbol] | 691307II | 0.169 | 0.365 | −0.047 | 0 | 0.194 | −0.005 | 2.61 | 0.265 | −0.42 | −0.137 | 0.192 | −0.127 | −0.006 | −0.126 | −0.056 |
| NotFound [symbol] | 691444II | 1.879 | −0.4825 | −0.2635 | −0.2545 | 0.0295 | −0.4305 | 0.4235 | −0.6405 | −0.34 | −0.2125 | −0.5955 | 0.1765 | 0.2355 | 0.4345 | 3.562 |
| NotFound [symbol] | 691485II | 0.7805 | 0.1025 | 0.2495 | −0.1885 | 0.4295 | −0.8605 | −0.3705 | 1.318 | −0.39 | 0.0105 | −0.7925 | −0.0275 | −0.0105 | −0.0805 | −0.6035 |
| NotFound [symbol] | 691652II | 0.933 | −0.401 | 0.179 | −0.333 | 1.112 | 0.453 | 1.412 | 0.441 | −0.41 | −0.507 | 0.125 | 1.39 | −0.046 | −0.405 | 0.901 |
| NotFound [symbol] | 691783II | 0.8475 | −0.2935 | −0.0905 | −0.1765 | 0.0045 | −0.0775 | 0.1155 | −0.1345 | −0.24 | −0.1595 | −0.0045 | 0.1155 | −0.0295 | −0.1085 | 0.3155 |
| NotFound [symbol] | 692077II | −0.0085 | −0.2025 | 0.1225 | 0.0175 | −0.1495 | 0.2155 | 0.3245 | 0.0085 | 0.44 | −0.4945 | 0.1865 | 0.2015 | 0.0615 | 0.0665 | −0.1135 |
| NotFound [symbol] | 692188II | 0.617 | 0.915 | −0.284 | 1.141 | 0.916 | 0.749 | 0.029 | 0.105 | 0.33 | −0.225 | −0.27 | −0.273 | −0.417 | 0.405 | 1.155 |
| NotFound [symbol] | 602468II | −0.155 | 0.287 | 0.861 | 0.371 | −0.071 | 0.116 | −1.082 | 0.874 | 0.37 | −0.982 | −0.546 | 0.796 | 0.287 | −1.171 | 0.439 |
| NotFound [symbol] | 692727II | 0.616 | −0.065 | 1.181 | −0.153 | −0.304 | 0.23 | −0.07 | 0.039 | −0.48 | 0.132 | 0.127 | −0.515 | 1.088 | 0.522 | 0.278 |
| NotFound [symbol] | 692860II | −0.53 | −1.752 | −0.918 | −0.147 | −0.534 | −0.727 | −0.793 | −0.852 | 0.86 | −0.464 | −1.84 | −0.508 | −1.111 | −1.328 | −0.316 |
| NotFound [symbol] | 692931II | 0.081 | −0.64 | −2.031 | 0.3 | −0.359 | −0.311 | −1.026 | −0.708 | 0.96 | −1.351 | −0.631 | −0.971 | 0.543 | 0.557 | −0.952 |
| NotFound [symbol] | 693121II | 2.511 | 0.958 | 2.645 | 3.463 | 2.508 | 2.028 | −0.226 | 1.183 | 0.44 | 1.406 | 2.106 | 1.69 | 1.979 | 2.499 | 1.832 |
| NotFound [symbol] | 693387II | 0.059 | −1.04 | −1.346 | 1.213 | 0.971 | 0.632 | −0.494 | −1.249 | 0.27 | −0.627 | 0.588 | −0.663 | −1.536 | 0.088 | −0.25 |
| NotFound [symbol] | 693511II | 1.09 | 2.264 | 0.381 | 3.114 | 0.888 | 2.797 | 0.604 | −0.916 | 0.87 | 0.37 | 2.742 | 1.116 | 1.612 | 1.146 | −0.256 |
| NotFound [symbol] | 693741II | −0.483 | −0.119 | 0.429 | −0.576 | −0.517 | −0.843 | −0.236 | 0.839 | −0.39 | −0.487 | −0.111 | −1.147 | −0.317 | 1.462 | 0.529 |
| NotFound [symbol] | 694011II | −0.19 | 0.645 | 0.439 | −0.257 | 0.077 | 3.597 | 2.186 | 3.571 | 0.97 | −0.338 | 2.079 | 3.562 | 1.073 | 2.084 | 1.806 |
| NotFound [symbol] | 694287II | 0.18 | −0.391 | −0.085 | 5.727 | 2.238 | 4.317 | 0.288 | −0.034 | 3.34 | 0.083 | 1.69 | −0.039 | 0.207 | −0.005 | 0.228 |
| NotFound [symbol] | 694458II | 0.0515 | −0.8165 | 0.0005 | −0.0005 | −1.877 | −0.0915 | 0.1465 | 0.0075 | −0.27 | −0.0785 | −0.0365 | 0.0705 | −0.1885 | 0.1135 | 0.0575 |
| NotFound [symbol] | 605199II | −0.381 | −0.741 | 1.01 | −1.071 | 0.38 | −2.431 | 1.094 | 1.195 | 0.21 | −2.01 | −0.398 | 1.875 | −1.873 | 0.27 | 0.513 |
| NotFound [symbol] | 695260II | −0.115 | 0.798 | −0.618 | −0.542 | −0.462 | −0.398 | −0.931 | 0.532 | −0.28 | −0.514 | −0.404 | −0.27 | −0.315 | −1.29 | −1.1 |
| NotFound [symbol] | 695667II | 1.002 | 1.648 | 0.528 | 0.735 | 0.474 | 1.45 | 0.962 | 0.398 | −0.09 | 0.757 | 1.469 | 0.625 | 0.645 | 0.458 | 1.584 |
| NotFound [symbol] | 686120II | 0.138 | 0.77 | 0.718 | 0.056 | −0.221 | 0.561 | 0.257 | −0.125 | −0.23 | 0.581 | −0.691 | 1.353 | 0.076 | 0.213 | 0.515 |
| NotFound [symbol] | 696388II | −0.615 | 0.773 | −2.528 | −1.209 | 1.618 | −2.194 | −1.314 | −0.138 | −0.38 | −1.998 | −1.986 | −0.609 | −2.078 | −1.833 | −2.589 |
| NotFound [symbol] | 696612II | −0.568 | 0.39 | −0.957 | 0.219 | −0.502 | −0.406 | −0.551 | −0.921 | −0.05 | 0.284 | −0.691 | −0.896 | 0.145 | −0.826 | −0.424 |
| NotFound [symbol] | 698792II | 0.705 | 0.196 | −0.953 | 1.188 | 0.201 | 1.07 | 0.317 | −0.541 | 0.07 | −0.013 | 0.703 | −0.049 | 0.956 | 0.22 | 0.147 |
| NotFound [symbol] | 697127II | 0.1375 | 0.2495 | −0.5695 | −0.1595 | 0.2525 | 0.0335 | 0.2865 | 0.3015 | 0.22 | 0.1585 | −0.3735 | 0.3455 | −0.3155 | 0.7245 | 0.1775 |
| NotFound [symbol] | 697197II | 0.723 | −0.055 | 1.468 | 0.058 | 3.136 | −0.032 | 0.169 | 0.021 | −0.06 | 0.256 | −0.014 | 0.138 | 0.451 | −0.236 | −0.158 |
| NotFound [symbol] | 697297II | 0.346 | 0.008 | 0.314 | −0.144 | 0.358 | −0.893 | −0.606 | 1.095 | −0.27 | −0.453 | −0.587 | 0.057 | 0.001 | −0.186 | −0.738 |
| NotFound [symbol] | 697403II | −0.658 | −0.184 | 0.226 | −0.437 | −0.309 | −0.681 | 1.539 | −0.391 | −0.08 | −0.259 | −0.63 | 2.042 | 0.069 | −0.704 | 0.373 |
| NPDC1 | 682819II [symbol] | 0.1735 | 0.0545 | −2.677 | 0.9465 | 1.363 | 0.5095 | −0.1275 | −2.289 | 0.68 | −2.719 | −0.2535 | −1.868 | −0.7335 | 0.1835 | −2.775 |
| ABAT | 682378II [symbol] | −0.71 | −0.17 | −1.718 | −0.198 | 2.101 | 1.742 | −2.210 | −2.788 | 2.15 | −1.856 | 1.885 | −1.976 | 0.743 | −1.355 | −1.937 |
| NPEPPS | 687914II [symbol] | −1.009 | 0.018 | −0.215 | −0.14 | 0.1 | 0.372 | −0.591 | 0.124 | 0.34 | 0.218 | −0.244 | −0.816 | −0.389 | −0.65 | 0.05 |
| NPY | 683068II [symbol] | −0.138 | 0.939 | −1.308 | −0.927 | 0.644 | −0.837 | −2.531 | −0.603 | −0.62 | −2.725 | 0.23 | −1.755 | 0.212 | −0.494 | −2.259 |
| NCO3A2 | 697754II [symbol] | 1.178 | 1.484 | −0.145 | 2.448 | 1.589 | 1.79 | 0.215 | 0.899 | 0.37 | 1.901 | 1.682 | 1.848 | 1.146 | 1.916 | 0.449 |
| NRAS | 695502II [symbol] | −0.3685 | −0.4395 | 0.8085 | 0.0065 | −0.6955 | −0.0065 | 0.8235 | 0.3565 | −0.32 | −0.6955 | −0.3545 | 0.3685 | −0.0235 | −0.6825 | 0.1995 |
| NRIP1 | 684742II [symbol] | −0.7187 | 0.6443 | −1.737 | −0.1937 | 0.6633 | 0.4093 | −1.614 | −0.0998 | 1.02 | 0.7413 | 0.5543 | 0.1443 | 1.028 | −0.7627 | −0.2947 |
| NTN4 | 686487II [symbol] | −2.377 | −2.057 | −2.951 | 0.5685 | 0.9135 | 0.7005 | −2.749 | −2.542 | 1.69 | −3.694 | 1.14 | −0.7775 | −2.364 | 0.6075 | −1.706 |
| NUCB2 | 681359II [symbol] | 0.3445 | 0.2835 | −1.769 | 1.428 | 1.359 | −0.1275 | −1.160 | −0.7865 | −0.09 | −1.115 | 0.6535 | −0.8685 | 0.6335 | 0.6435 | −0.0295 |
| NUDT5 | 695608II [symbol] | −0.669 | −0.463 | 1.02 | −0.512 | −0.874 | −0.383 | 0.077 | 1.26 | −0.39 | −0.019 | −0.49 | 0.31 | −0.065 | −0.64 | 0.545 |
| NUDT6 | 686517II [symbol] | −1.086 | −0.377 | −0.77 | 0.348 | 0.665 | −0.03 | −1.025 | −0.644 | 0.29 | −0.503 | 0.243 | −0.794 | 0.46 | −1.605 | 0.74 |
| OCIAD2 | 695560II [symbol] | 1.556 | 0.083 | 0.976 | 0.331 | 1.224 | 0.827 | 0.462 | 0.898 | −0.03 | −2.593 | 0.395 | 1.374 | 2.435 | 0.884 | 0.691 |
| OFD1 | 683518II [symbol] | −0.645 | 0.348 | 1.176 | 0.676 | 0.473 | 0.287 | −0.229 | −1.59 | 0.36 | −1.976 | −0.656 | −1.39 | 0.199 | 0.491 | 0.037 |
| OGFRL1 | 689994II [symbol] | −0.302 | −0.092 | 1.239 | −0.284 | −0.001 | −0.061 | 0.109 | 1.841 | 0.26 | −0.694 | 0.53 | 4.002 | −0.517 | −0.501 | 2.491 |
| OPLAH | 694256II [symbol] | 0.872 | 1.927 | 0.106 | 0.59 | 0.803 | 1.041 | 2.102 | 2.182 | −0.37 | −0.036 | 0.819 | 2.341 | −0.099 | 0.466 | 0.012 |
| ORMDL3 | 663021II [symbol] | 0.592 | 0.193 | −0.627 | −0.011 | 0.352 | 0.042 | −0.618 | −0.522 | −0.08 | −0.272 | 0.011 | 0.044 | −0.43 | 1.285 | −0.545 |
| OSBPL3 | 691049II [symbol] | 1.505 | −0.5435 | −0.9035 | −0.7325 | −0.0115 | −0.0705 | 1.804 | 1.011 | −0.6 | 0.4865 | −0.9725 | −0.1815 | 0.0115 | −0.6415 | 0.6005 |
| OSR2 | 681734II [symbol] | 0.1958 | 1.167 | 0.0428 | 1.501 | −0.5622 | −0.8882 | 0.8548 | −1.217 | −0.73 | −0.7352 | 0.3458 | 3.772 | −0.3802 | −0.3812 | −0.6452 |
| OSTF1 | 684940II [symbol] | 0.262 | 0.597 | 0.326 | −0.051 | 0.16 | 0.118 | 1.611 | 1.132 | −0.61 | 0.003 | 0.185 | 0.304 | −0.138 | 0.095 | 1.357 |
| P2RX1 | 681440II [symbol] | 1.609 | 1.436 | −0.028 | −0.061 | 1.852 | −0.255 | 0.534 | −0.287 | −0.19 | −0.328 | 0.117 | 0.21 | −0.125 | 0.007 | 1.801 |
| P2RY2 | 696219II [symbol] | −1.161 | 0.13 | −0.411 | −0.885 | −0.535 | −1.02 | −1.669 | −0.753 | 0.1 | −0.443 | −1.637 | −1.193 | 2.441 | −0.728 | −0.22 |
| PACAP | 697667II [symbol] | 0.31 | −2.235 | −3.014 | −2.5 | −0.698 | −2.785 | −1.973 | −3.071 | −0.84 | −2.644 | −1.069 | −1.717 | 0.013 | −0.985 | 0.047 |
| PADI2 | 688464II [symbol] | 1.859 | −0.6385 | 0.5505 | −0.1275 | −0.5545 | −0.5545 | 2.729 | 1.93 | −0.16 | 0.5215 | −0.1725 | 1.101 | −0.0925 | 0.7365 | 2.313 |
| PAPD5 | 688131II [symbol] | 0.5255 | 1.104 | −0.1125 | −0.7245 | 0.7225 | 0.9225 | 0.5705 | −0.0265 | 0.13 | 0.1485 | 0.3655 | 0.0985 | −0.0215 | −0.3725 | −0.1015 |
| PAPSS2 | 688187II [symbol] | 0.4605 | 0.4345 | 1.658 | −1.001 | −2.126 | 0.3236 | −0.6235 | 1.457 | −0.43 | −1.202 | −0.2445 | −0.6275 | −0.8735 | 1.077 | 0.2855 |
| PARD6A | 692612II [symbol] | 0.444 | −0.219 | −0.046 | 0.625 | 1.665 | 0.964 | 0.239 | −0.456 | −0.21 | −0.603 | 0.374 | −0.091 | 0.256 | −0.175 | −0.058 |
| PARP1 | 683432II [symbol] | −0.1475 | 0.2525 | 1.413 | −0.1355 | 0.4155 | −0.5755 | 0.6025 | 0.7165 | −0.19 | −0.0595 | −0.2305 | 0.5045 | 0.2155 | 0.1845 | 0.2375 |
| PARP9 | 695188II [symbol] | 0.325 | 1.544 | 0.445 | 0.973 | 1.148 | 2.073 | 1.395 | −0.08 | −0.05 | 0.577 | 0.942 | 1.935 | 0.143 | 1.337 | 0.673 |
| PAXIP1L | 684118II [symbol] | −0.4328 | −0.2018 | 3.339 | −0.5648 | −0.4258 | 1.057 | 0.9922 | 0.7332 | −0.02 | −1.503 | 1.694 | 3.063 | −0.9718 | −1.421 | 1.333 |
| PBX3 | 692245II [symbol] | −0.562 | 0.128 | −1.846 | 2.026 | 0.584 | 0.036 | −0.787 | −0.591 | 0.15 | −1.712 | 0.776 | −0.691 | −1.442 | −0.532 | −0.853 |
| PCDHB1 [symbol] | 681640II [symbol] | 0.0985 | −0.7045 | 0.2895 | −0.1915 | 0.1235 | −0.7305 | −0.2395 | 2.412 | 0.23 | −0.7285 | −0.2945 | 0.7165 | −0.0015 | −0.7655 | 0.9915 |
| PCDHB16 | 697521II [symbol] | −1.995 | 0.568 | −0.959 | −2.49 | 1.603 | −0.507 | 0.454 | −1.658 | 0.8 | −0.949 | 0.403 | −0.774 | −0.104 | −0.293 | 0.075 |
| PCDHB2 | 684692II [symbol] | 0.912 | 0 | −1.488 | 0.799 | 1.343 | −1.191 | −1.219 | 3.913 | 0.6 | −1.004 | −0.456 | 2.291 | −0.809 | −1.133 | 1.692 |
| PCM1 | 685108II [symbol] | −0.5415 | −1.141 | 0.9375 | 1.062 | 0.5985 | −0.8655 | −0.0095 | −0.5625 | 1 | −0.2865 | 0.3535 | −1.318 | −0.3155 | −1.18 | −0.2825 |
| PCNA | 683820II [symbol] | −0.371 | −1.99 | 0.23 | 0.565 | 0.64 | 0.62 | 0.007 | 0.889 | −0.41 | 1.322 | −0.95 | 1.56 | −1.34 | −1.13 | 0.513 |
| PDCD1 | 687556II [symbol] | 0.858 | −0.301 | −0.335 | −0.303 | 0.532 | −0.304 | 0.383 | −0.241 | −0.47 | −0.615 | 0.182 | −0.004 | 0.453 | 0.004 | 0.839 |
| PDCD10 | 694505II [symbol] | −0.898 | −0.523 | −0.886 | −0.351 | −1.155 | −1.274 | −0.34 | 0.792 | −0.16 | −0.351 | −0.46 | −1.263 | −1.381 | 0.417 | 0.752 |
| PDCD4 | 692083II [symbol] | −0.514 | −0.405 | 0.002 | −0.232 | −0.178 | −0.537 | −0.19 | −0.236 | 1.46 | 0.018 | −0.351 | −1.105 | −0.507 | −0.22 | 0.003 |
| PDCD5 | 682521II [symbol] | −0.521 | 0.611 | 1.98 | 0.194 | 0.329 | −0.094 | 0.522 | 1.018 | −0.27 | 0.3 | 0.239 | −0.37 | 1.849 | 0.196 | 1.224 |
| PDCD8 | 697583II [symbol] | 0.309 | 0.667 | 0.462 | −0.139 | 0.726 | 0.088 | 0.048 | −0.993 | −0.28 | −0.315 | −0.063 | −0.297 | 0.058 | −0.023 | 0.09 |
| PDE4D1P | 689720II [symbol] | 0.5505 | 0.8825 | −1.498 | 1.144 | 0.7885 | −0.5175 | −0.1685 | −0.2675 | −0.17 | 0.6125 | 1.029 | 2.657 | 0.2805 | −0.2765 | 0.3135 |
| PDHA1 | 684251II [symbol] | 1.948 | −1.116 | −0.196 | 0.554 | 1.013 | −1.386 | −0.163 | −1.02 | −0.29 | 0.074 | −0.576 | 0.26 | 0.435 | −0.194 | 0.988 |
| PECI | 689878II [symbol] | 0.015 | 0.445 | 0.134 | 0.479 | 0.691 | 0.163 | −1.96 | −1.161 | 0.63 | −1.472 | 1.07 | −0.875 | 0.279 | −0.046 | −1.195 |
| PEG10 | 687554II [symbol] | 2.096 | −2.503 | 0.6945 | −2.266 | −2.721 | 0.2975 | 1.435 | 1.029 | 2.23 | −1.282 | 0.8765 | 0.1785 | 2.371 | −1.516 | −0.7405 |
| PER2 | 688213II [symbol] | −0.1658 | 0.2282 | 0.1472 | 0.3672 | 1.164 | 0.3942 | −1.697 | −0.6398 | 0.78 | −0.4948 | 1.079 | −0.6458 | −0.7578 | −0.1958 | −0.9008 |
| PERP | 683121II [symbol] | 0.907 | −0.025 | 0.338 | −0.592 | −0.829 | −1.11 | −0.086 | 0.763 | 0.68 | 0.025 | 0.262 | 0.123 | −2.059 | 0.382 | 1.748 |
| PEX1 | 686946II [symbol] | −0.1345 | −0.3585 | 0.7665 | 0.6825 | −0.0245 | 0.1755 | 0.9655 | −0.0115 | 0.41 | −0.6805 | −0.2425 | −0.9975 | 0.0225 | 0.0115 | −0.4615 |
| PFAAP5 | 683100II [symbol] | −0.555 | −0.506 | 0.085 | −0.004 | −0.607 | −3.034 | 0.135 | −0.073 | 0.98 | −0.525 | −0.504 | −0.791 | −0.157 | −0.603 | 0.883 |
| PFDN2 | 687458II [symbol] | 0.272 | 0.517 | 0.61 | 0.177 | 0.236 | −0.25 | 0.34 | 1.036 | −0.22 | 0.088 | −0.303 | 1.176 | 0.984 | 0.276 | 1.12 |
| PGM1 | 681276II [symbol] | −0.4795 | −0.8405 | −0.2895 | −0.8445 | 0.0635 | 0.6846 | −0.0065 | 0.7105 | 0.52 | 0.1965 | 1.671 | 1.075 | −0.6045 | −0.1585 | −0.2585 |
| PGR1 | 689407II [symbol] | −0.23 | 0.751 | −0.779 | 0.047 | 0.076 | 0.817 | 0.078 | −1.348 | 0.43 | −0.623 | 0.662 | −0.415 | 0.378 | −0.519 | −0.831 |
| PHAX | 692593II [symbol] | −0.419 | 0.012 | −1.091 | −0.548 | −0.182 | −0.879 | −0.507 | −0.369 | −0.22 | 0.019 | −0.603 | −0.885 | 0.123 | −0.962 | −0.193 |
| PHGDH | 682626II [symbol] | 0.15 | −1.292 | 0.386 | −0.503 | −0.601 | −1.135 | 0.088 | 0.582 | −0.19 | 0.769 | −1.183 | 1.472 | 0.211 | −0.088 | 1.078 |
| PHYH | 689499II [symbol] | −0.346 | −1.005 | 1 | −0.418 | 0.031 | 1.388 | 0.788 | 0.243 | −0.52 | −0.031 | 0.303 | 0.308 | 0.765 | −1.026 | 1.488 |
| PIB5PA | 691558II [symbol] | −0.397 | −0.005 | −2.788 | 0.719 | 0.333 | 1.597 | −2.388 | −1.981 | 1 | −2.654 | 0.21 | −2.071 | −1.195 | 0.12 | −1.718 |
| PIGT | 688446II [symbol] | −0.3085 | 0.5915 | −0.2325 | 1.018 | 2.544 | 0.2975 | −0.4335 | −0.4455 | −0.24 | 1.031 | 0.4765 | −0.2705 | −0.1245 | −0.4216 | −0.4215 |
| PIM2 | 697074II [symbol] | −1.159 | −1.19 | 0.032 | −0.114 | 1.128 | −0.312 | −0.273 | 0.905 | −0.63 | −1.57 | −0.032 | −0.152 | −0.864 | −1.161 | 0.06 |
| PISD | 696650II [symbol] | 0.668 | 0.567 | −0.252 | 0.853 | 0.81 | 1.992 | −0.175 | −0.32 | 0.1 | 0.826 | 1.353 | −0.013 | −0.16 | −0.086 | −0.242 |
| PITPNC1 | 684234II [symbol] | 0.745 | −0.117 | −0.155 | −0.377 | −0.006 | 0.389 | 0.041 | 0.378 | −0.16 | −0.303 | −0.379 | 0.248 | −0.225 | 2.398 | 0.159 |
| PKIB | 681591II [symbol] | −0.345 | −0.273 | 0.277 | 3.269 | −1.07 | 0.752 | −3.539 | −0.727 | 1.96 | 2.017 | 2.753 | −1.332 | 3.149 | −1.006 | −0.623 |
| PKMYT1 | 686037II [symbol] | 1.06 | 0.5515 | 0.8505 | −0.4935 | −0.3405 | 0.2945 | 2.046 | 0.9765 | −1.11 | 1.301 | −0.7115 | 0.3555 | 0.8195 | −0.2225 | 0.9665 |
| PLA2G4B | 695710II [symbol] | −0.1965 | 0.0135 | −1.006 | −1.06 | −1.087 | 1.173 | −0.0135 | −0.7605 | −0.02 | 2.243 | 0.8385 | 1.142 | 0.6125 | 0.3055 | 0.3526 |
| PLAC8 | 692243II [symbol] | −3.344 | −4.988 | −3.417 | −3.819 | −4.32 | −4.218 | −1.271 | −3.715 | 0.41 | −0.28 | −3.168 | −3.393 | −1.956 | −2.356 | −0.128 |
| PLCB1 | 681242II [symbol] | −0.748 | 1.734 | −1.097 | −2.351 | −1.293 | −2.69 | −2.711 | −3.449 | −0.27 | −0.356 | −1.282 | −3.087 | −0.213 | −0.572 | −3.133 |
| PLCXD1 | 686574II [symbol] | 3.154 | 0.534 | −1.819 | −0.796 | 1.773 | −2.249 | 1.313 | −1.728 | −0.04 | −0.888 | 0.139 | 1.247 | 1.302 | 1.271 | 2.642 |
| PLEK2 | 686964II [symbol] | 1.267 | −0.4215 | −0.8185 | 0.6855 | −0.3155 | −0.6475 | −0.7985 | −0.8685 | −0.32 | 1.021 | −0.2575 | −0.7105 | −0.7835 | 0.2415 | 0.2065 |
| PLOD | 690311II [symbol] | −0.0475 | −0.6575 | 0.2995 | −0.2005 | −0.1785 | −0.4055 | 0.4335 | 1.205 | 0.02 | 0.8305 | −0.4365 | 2.04 | 0.2825 | −0.2855 | −0.5085 |
| PNAS-4 | 695276II [symbol] | 0.207 | −0.576 | 0.508 | −0.495 | −1.226 | −0.962 | 0.872 | 1.14 | 0.07 | −0.067 | −0.737 | 0.049 | −0.784 | 0.449 | 0.662 |
| PNLIPRP [symbol] | 692823II [symbol] | −0.156 | 0.646 | −0.493 | 0.215 | 0.782 | 1.031 | −0.404 | −0.238 | −0.05 | −1.025 | 0.588 | −0.613 | −0.097 | 0.107 | −0.304 |
| PNMA3 | 695191II [symbol] | 0.1295 | −0.1835 | −0.3205 | 3.218 | 0.3655 | −0.1665 | −0.7345 | −0.0785 | −0.16 | −0.2635 | 2.323 | 0.0675 | −0.1045 | −0.1775 | −0.2535 |
| POLB | 688699II [symbol] | 2.088 | 1.575 | 0.221 | −0.382 | −0.08 | −0.628 | 0.556 | −0.01 | −0.24 | −0.593 | 0.411 | −0.241 | 0.074 | 1.152 | 2.216 |
| POLR2F | 695452II [symbol] | −0.649 | −0.755 | −0.366 | −0.547 | −0.737 | 0.026 | 0.426 | 0.321 | 0 | 0.684 | −0.312 | 0.607 | −0.042 | −1.245 | −0.114 |
| POLR2H | 692429II [symbol] | 0.9065 | −0.6015 | 0.5505 | −0.9205 | 0.2005 | −0.2305 | −0.7065 | 0.4135 | 0.33 | −0.2945 | −0.5185 | 0.3005 | −0.5595 | 0.1745 | 0.0485 |
| POMC | 653917II [symbol] | −0.0035 | 0.2385 | −0.1845 | −0.3545 | 0.0065 | −0.4385 | 0.4895 | 0.8025 | −0.28 | 1.426 | −0.2725 | 0.6025 | 0.0935 | −0.3615 | 0.7725 |
| POP4 | 687322II [symbol] | 0.001 | −0.199 | 1.097 | 0.038 | −0.471 | −0.354 | −0.361 | 0.071 | −0.27 | 0.786 | −0.222 | 2.501 | 1.273 | 0.067 | 0.234 |
| POP7 | 688981II [symbol] | 0.0745 | −0.2645 | 0.1535 | 0.1775 | 0.4475 | 0.1645 | 0.9775 | 0.5255 | −0.34 | 0.8055 | 0.1555 | 0.1925 | 0.1225 | −0.2575 | −0.3695 |
| PP1057 | 694694II [symbol] | 2.019 | 1.431 | 2.411 | 1.805 | 1.547 | 2.017 | 0.156 | 1.213 | 0.63 | 1.577 | 1.803 | 1.307 | 1.819 | 2.548 | 0.317 |
| PPFIA1 | 684385II [symbol] | 0.0235 | 0.2145 | 0.3355 | −0.2185 | 0.3175 | −0.0205 | −0.4075 | −0.4345 | 0.36 | −0.4965 | 0.4255 | 0.5605 | 2.388 | 0.0305 | 0.0175 |
| PPFIBP2 | 688244II [symbol] | 0.88 | −0.292 | 1.288 | 0.107 | −0.237 | 0.715 | −0.02 | 0.031 | 0.54 | −0.602 | −0.246 | −0.332 | −0.179 | 1.238 | 0.189 |
| PPGB | 696506II [symbol] | 0.37 | 0.833 | −0.988 | 0.005 | −0.136 | 0.036 | −0.066 | −0.493 | −0.53 | −0.299 | 0.387 | 0.964 | −0.327 | −0.374 | 0.03 |
| PPHLN1 | 681157II [symbol] | 0.4155 | 0.0325 | −0.5845 | 0.3255 | 0.2405 | −0.5415 | −0.6995 | −0.5485 | −0.06 | −0.0655 | −0.4015 | −0.1505 | 0.0345 | −0.6015 | 0.5485 |
| PPIF | 685607II [symbol] | 0.213 | −0.099 | 1.689 | −0.469 | 0.021 | −0.633 | −0.288 | 1.432 | −0.76 | 1.174 | 2.632 | 0.273 | −0.828 | 0.536 | 0.674 |
| PPIL1 | 693349II [symbol] | −1.466 | −1.22 | −0.691 | −1.917 | −1.34 | −1.446 | −1.423 | −0.624 | 0 | 0.161 | −1.339 | −1.27 | −0.781 | −1.238 | −0.358 |
| PPOX | 686128II [symbol] | −0.155 | 0.0667 | 0.343 | 0.425 | 0.595 | −0.195 | −0.115 | −0.525 | 0.21 | −0.608 | −0.26 | 0.284 | 0.414 | −0.078 | 0.173 |
| PPP1R16 [symbol] | 684680II [symbol] | −0.012 | 0.461 | 0.65 | −0.021 | −0.38 | 1.01 | 1.286 | 1.58 | 0.29 | −0.871 | −0.504 | 1.339 | 0.251 | −0.722 | −0.27 |
| PPP1R3C | 688073II [symbol] | −1.06 | −0.7895 | −2.277 | 1.711 | 1.235 | −0.4055 | −0.9295 | −1.452 | 1.76 | −1.539 | 2.71 | −1.159 | 0.0725 | −1.323 | −1.275 |
| PPP2R2D | 689931II [symbol] | 0.2275 | 0.3975 | −0.1515 | 0.3345 | 0.0315 | 0.3455 | 0.1045 | −0.5305 | 0.03 | −0.3975 | −0.0705 | 0.4265 | 0.2415 | −0.1585 | −0.9605 |
| PPP2R5A | 686848II [symbol] | −0.4735 | −0.1785 | −0.4635 | 0.5235 | −0.1675 | −0.2695 | −0.3595 | 0.2905 | 0.28 | 1.557 | −0.3135 | −0.1165 | −0.3655 | −0.2475 | 0.8925 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| PRAME | 690640II [symbol] | −0.0805 | 2.018 | 2.206 | −0.4305 | −0.9355 | 0.0805 | 1.646 | 2.753 | 0.33 | −0.2455 | −0.3405 | 2.649 | −0.4505 | 1.311 | −0.4895 |
| PRC1 | 685404II [symbol] | 0.5055 | 0.4215 | 0.0205 | −0.1335 | −0.0055 | −0.0725 | 0.8505 | 0.9885 | −0.93 | 1.247 | −0.0145 | 0.3745 | 0.0505 | −0.4745 | 0.6155 |
| PRIM2A | 687823II [symbol] | 0.219 | −0.789 | 1 | 0.031 | −1.759 | 0.141 | 0.694 | 1.127 | −0.22 | 1.16 | −0.476 | 0.69 | 0.101 | −0.223 | 1.14 |
| PRKACB | 696518II [symbol] | 0.184 | 0.736 | −0.325 | −0.083 | 2.46 | 0.011 | −0.213 | −0.385 | −0.31 | −0.529 | 0.124 | 0.031 | −0.309 | −0.246 | −0.166 |
| PRKCB1 | 694132II [symbol] | −1.314 | −4.887 | −3.335 | −2.564 | −3.055 | −1.876 | −0.723 | −3.027 | −0.22 | −2.548 | −1.8 | −2.095 | −1.371 | −2.252 | −0.848 |
| PRKCI | 686943II [symbol] | 0.2142 | 0.4552 | −0.3278 | −0.5188 | −0.4778 | −0.6818 | 0.0853 | 1.178 | −0.44 | −0.0718 | 0.7142 | −1.108 | −0.7328 | 0.6772 | 0.8622 |
| PRKRIR | 684358II [symbol] | −0.02 | −0.153 | 1.157 | −0.286 | 0.005 | −0.093 | −0.062 | 0.99 | 0.35 | 0.699 | 0.039 | −0.058 | −0.96 | −0.005 | 0.429 |
| PRKWNK [symbol] | 683385II [symbol] | −1.015 | −2.128 | −1.084 | −1.411 | −2.367 | −1.252 | −0.863 | −0.698 | −0.07 | −0.239 | −1.871 | −0.891 | −1.552 | −1.56 | −0.454 |
| PRKX | 686136II [symbol] | −0.033 | 0.505 | −1.15 | −0.106 | 0.279 | −0.321 | −0.002 | −1.268 | −0.71 | −1.428 | −0.214 | −1.256 | 0.292 | −0.632 | −0.65 |
| PRNP | 692320II [symbol] | −2.144 | −1.362 | 0.178 | −3.134 | −1.218 | −2.326 | −0.903 | −0.027 | 1.37 | −2.176 | −1.664 | −1.568 | −1.672 | −2.493 | −1.014 |
| PROL5 | 684331II [symbol] | 1.785 | 0.055 | −0.326 | −0.323 | 0.271 | 0.214 | −0.08 | −0.435 | −0.02 | 0.017 | 0.334 | 0.222 | −0.017 | −0.141 | 1.523 |
| PRPF18 | 685943II [symbol] | −0.155 | −0.669 | 0.481 | −0.135 | −0.879 | −0.182 | 0.075 | 1.011 | 0.07 | 0.703 | −0.011 | −0.481 | 0.171 | −0.168 | 1.339 |
| PRPSAP [symbol] | 681061II [symbol] | −0.2535 | 1.33 | 0.7345 | 0.1335 | 0.4615 | 0.0095 | −0.0685 | 1.111 | 0.27 | −0.6425 | 0.0705 | 2.264 | 0.4565 | −0.6715 | 0.6425 |
| PRSS12 | 692634II [symbol] | −1.489 | −2.302 | −1.051 | −1.615 | −2.401 | −1.937 | −1.31 | 1.807 | 0.73 | −0.279 | −2.379 | 2.018 | −1.461 | −1.371 | 1.101 |
| PRSS16 | 695121II [symbol] | 0.54 | −0.69 | 1.128 | −0.751 | 1.022 | 1.791 | 0.93 | 0.792 | 0.6 | 0.184 | 0.526 | 0.796 | −0.951 | −0.516 | 1.874 |
| PSD3 | 688328II [symbol] | −0.097 | −0.313 | −1.53 | 2.714 | −0.228 | 0.194 | −1.309 | 0.097 | 0.96 | −1.615 | 0.573 | 1.733 | −1.495 | −1.573 | −2.035 |
| PSMA3 | 682239II [symbol] | 0.3395 | −0.7445 | 0.0175 | 0.0815 | −0.1315 | −0.4945 | 0.2785 | 0.3595 | −0.45 | 1.723 | 0.0145 | −0.8855 | 1.139 | 0.0955 | 0.8745 |
| PSMA7 | 694665II [symbol] | −0.241 | 0.337 | −0.025 | −0.583 | 0.331 | −0.662 | −0.399 | −0.034 | −0.85 | 1.143 | 0.121 | 0.435 | −0.264 | −0.966 | −0.469 |
| PSMC5 | 687471II [symbol] | 1.521 | 0.9305 | −0.8705 | −0.0105 | 0.0055 | 0.4445 | −0.2935 | −0.2125 | 0.09 | 0.3015 | −0.2245 | −0.2235 | 0.6885 | −0.6665 | −0.2555 |
| PSMC6 | 693728II [symbol] | −1.063 | −0.877 | −1.153 | −1.019 | −0.745 | −1.054 | −1.437 | −0.73 | 0.1 | 1.09 | −0.121 | −1.531 | −0.177 | 0.253 | −0.738 |
| PSMD12 | 697390II [symbol] | 0.858 | −0.327 | −0.579 | −0.245 | −0.138 | −0.807 | −0.235 | −0.404 | −0.16 | −1.447 | −0.443 | −0.319 | 0.502 | −0.308 | 0.568 |
| PSMD12 | 697530II [symbol] | −0.716 | −0.292 | −1.769 | −1.315 | −1.607 | −1.881 | −1.012 | −0.01 | −0.38 | −0.214 | −1.052 | −0.632 | −0.032 | −1.341 | −0.474 |
| PSMD3 | 681750II [symbol] | 0.409 | 0.173 | −0.753 | −0.836 | 0.029 | −0.708 | 0.839 | 0.038 | −0.59 | −0.245 | 0.02 | 0.279 | −0.135 | −0.272 | 0.116 |
| PSPH | 685810II [symbol] | −0.3 | 0.707 | −0.298 | 0.358 | 0.163 | 0.965 | −0.392 | −0.372 | 0.36 | −0.868 | 0.249 | −0.313 | 0.583 | −0.428 | −0.542 |
| PTD012 | 684256II [symbol] | 0.7645 | 0.5655 | 0.5105 | 0.7455 | 0.0445 | 0.1925 | 1.261 | −0.1825 | −1.05 | −1.012 | −0.1225 | −0.1595 | −0.4125 | −0.0725 | 0.2025 |
| PTGER3 | 685654II [symbol] | 0.118 | −0.72 | 0.006 | −0.211 | 0.711 | 0.018 | 0.037 | −0.265 | 0.57 | −0.131 | 1.208 | −0.246 | −0.348 | −0.143 | −0.204 |
| PTHLH | 683001II [symbol] | −1.901 | −2.746 | 1.032 | −1.44 | −0.071 | 3.487 | 0.811 | −1.115 | 1.27 | −0.414 | 1.071 | −0.026 | −0.463 | −1.052 | 0.365 |
| PTK6 | 690829II [symbol] | 0.805 | −0.28 | −0.847 | 0.569 | −0.493 | 0.253 | −0.399 | 0.053 | −0.21 | −1.244 | −0.38 | −0.146 | −0.642 | 0.434 | −0.695 |
| PTPN4 | 693676II [symbol] | 0.291 | 0.25 | 0.95 | 0.03 | 0.292 | 0.422 | −0.216 | 0.879 | −0.27 | −0.407 | −0.478 | −0.286 | −0.722 | −0.145 | −0.015 |
| PTPN7 | 687885II [symbol] | 1.023 | −0.1205 | −0.5975 | −0.7545 | 0.5475 | 0.1825 | 0.6495 | −0.6035 | −0.2 | −0.3165 | 0.1575 | −0.0415 | 0.2295 | 0.0445 | 0.9585 |
| PTPRF | 684441II [symbol] | −0.381 | 0.051 | −0.144 | 0.359 | 0.078 | 0.895 | 0.397 | 0.48 | −0.45 | 0.106 | −0.476 | 0.612 | −0.154 | −0.051 | 1.243 |
| PTPRN2 | 685456II [symbol] | −0.2195 | −0.2725 | −0.1605 | 3.799 | 0.6745 | 0.5415 | 0.0685 | −0.4525 | −0.55 | −0.2395 | 0.2335 | −0.0215 | 0.1415 | −0.6435 | −0.3515 |
| PTPRT | 694229II [symbol] | 0.374 | −0.183 | 1.026 | 1.647 | −0.078 | 1.197 | 0.435 | 0.059 | 0.89 | 0.072 | 1.893 | −0.915 | 0.737 | 0.258 | 0.034 |
| PTS | 691288II [symbol] | 0.102 | −0.015 | 0.251 | −0.099 | 0.797 | −0.228 | 0.819 | 1.69 | −0.17 | 0.032 | 0.072 | 0.124 | −0.039 | 0.015 | −0.479 |
| PTTG1 | 687187II [symbol] | 1.077 | 0.072 | 1.483 | −0.072 | −0.377 | −0.374 | 2.01 | 1.786 | −1.01 | 1.687 | −0.366 | 0.7 | 0.726 | −0.222 | 1.281 |
| PURB | 697166II [symbol] | −0.832 | −1.322 | −1.054 | −1.117 | −0.991 | −1.562 | −1.264 | −0.176 | −0.73 | 0.46 | −0.763 | −1.013 | −1.085 | 0.437 | −0.004 |
| PXMP2 | 694802II [symbol] | −0.935 | 0.166 | −1.627 | −0.497 | −0.642 | −0.114 | −0.649 | 0.415 | −0.03 | −0.328 | −0.462 | −0.374 | −0.397 | −1.965 | −0.3 |
| PYCARD | 696817II [symbol] | −0.14 | 0.38 | 0.164 | −0.519 | 0.373 | 0.89 | −0.017 | 0.187 | 0.16 | −0.631 | −0.526 | 0.026 | −0.333 | −0.428 | 0.451 |
| PYCR1 | 683284II [symbol] | 0.2435 | 0.8025 | 0.2965 | −0.3605 | 0.0205 | −1.04 | −0.2365 | 0.5985 | −0.66 | 0.5685 | −1.127 | 0.4595 | 0.5805 | 0.2495 | −0.6445 |
| QDPR | 695556II [symbol] | −2.105 | −1.157 | −0.92 | 2.74 | 1.735 | 0.588 | −0.723 | −0.723 | 1.62 | −1.767 | 1.721 | 0.326 | 0.74 | 0.374 | −1.283 |
| QPRT | 681328II [symbol] | 0.584 | 1.485 | 0.377 | −0.155 | 0.366 | −0.356 | −0.172 | 0.867 | −0.36 | 0.26 | −0.328 | 0.821 | 0.625 | −0.481 | 0.103 |
| QSCN6 | 691861II [symbol] | 0.636 | 0.026 | 0.263 | −0.208 | −0.026 | 0.514 | −0.762 | −0.723 | 0.41 | 1.344 | −0.235 | 1.162 | 0.335 | −1.373 | −0.988 |
| RAB11FIP | 697363II [symbol] | 0.174 | −0.551 | 0.75 | −2.627 | −0.092 | −1.628 | −0.077 | −0.024 | 0 | 1.007 | −1.028 | −0.299 | −0.483 | −0.303 | −0.434 |
| RAB11FIP | 692380II [symbol] | 2.606 | 0.749 | 1.3 | 0.224 | −1.283 | −0.52 | 1.127 | 3.487 | 0 | −0.79 | −0.259 | 3.659 | 0.496 | 0.791 | 2 |
| RAB13 | 681521II [symbol] | −0.1547 | 0.3343 | −0.1817 | 0.5293 | 0.6823 | 0.1263 | −0.3347 | 0.1433 | −0.01 | −0.9147 | −0.9267 | 0.6273 | −0.0068 | −0.0208 | −0.9097 |
| RAB27A | 689787II [symbol] | 1.104 | −0.3815 | −0.7145 | −0.5825 | 0.1195 | −0.4465 | 0.7355 | 1.427 | −0.05 | 1.498 | 0.1835 | −0.0425 | 0.9175 | 0.1175 | 0.2525 |
| RAB30 | 690906II [symbol] | 0.002 | 0.68 | −0.463 | 1.912 | 0.11 | 0.386 | −0.47 | −0.268 | −0.23 | −0.674 | −0.093 | −0.233 | −0.411 | 0.546 | 0.446 |
| RAB34 | 687074II [symbol] | −0.814 | −0.238 | 0.598 | −0.421 | −0.035 | 0.835 | −0.23 | 0.412 | 0.58 | −0.175 | −0.47 | 0.212 | −1.748 | 0.485 | −0.183 |
| RAB35 | 689469II [symbol] | 0.532 | 0.027 | −1.045 | −0.098 | −0.792 | −0.117 | 0.34 | 0.473 | −0.82 | 0.007 | −0.55 | 0.762 | 0.017 | −0.786 | 0.545 |
| RAB39B | 696999II [symbol] | −0.476 | −1.549 | −0.264 | −1.408 | 0.237 | 0.079 | −0.73 | −0.719 | −0.33 | −0.779 | 0.47 | −0.643 | −0.594 | −1.349 | −0.539 |
| RAB3A | 686794II [symbol] | 0.1255 | −0.0095 | −0.2045 | 0.0135 | 0.2895 | −0.0785 | −0.3365 | −0.0055 | 0.3 | −0.1465 | −0.2215 | −0.0015 | 0.0425 | 0.0015 | −0.1555 |
| RAB3IP | 690420II [symbol] | 0.0075 | −0.6655 | −0.5345 | 0.3125 | −0.8745 | 0.0095 | 0.3725 | 0.0675 | 0.1 | −1.794 | −1.474 | −1.138 | −0.6705 | −0.1635 | 1.682 |
| RABEP1 | 687547II [symbol] | −0.6175 | 0.0745 | −0.8635 | 2.051 | 1.407 | 1.134 | −1.134 | −1.084 | 0.84 | −0.5205 | 1.231 | −1.07 | −0.0745 | 1.433 | 0.4665 |
| RABGAP1 | 694298II [symbol] | 0.59 | −0.173 | 0.138 | 0.86 | 0.67 | −0.43 | 0.73 | 0.725 | −0.26 | −0.728 | −0.074 | 0.09 | −0.21 | 0.499 | 2.322 |
| RACGAP1 | 690792II [symbol] | −2.932 | −3.677 | −1.273 | −2.357 | −0.878 | −2.085 | −0.526 | −1.777 | −0.79 | −1.314 | −2.62 | −0.427 | −0.945 | −3.281 | −0.846 |
| RAD17 | 691216II [symbol] | −0.464 | −0.237 | −0.477 | 0.421 | 0.276 | 0.008 | 0.091 | −0.023 | 0.11 | −0.513 | 0.216 | −0.909 | 0.188 | −0.485 | −0.158 |
| RAD51 | 692122II [symbol] | −1.409 | −1.084 | −1.065 | −2.419 | −2.134 | −1.5 | 0.343 | 0.012 | −1.23 | 0.629 | −1.847 | −1.167 | −1.614 | −2.083 | 0.321 |
| RAD51AP | 687081II [symbol] | −0.077 | −0.2225 | 2.544 | −0.218 | −0.693 | −0.491 | 0.366 | 2.031 | −0.3 | 1.475 | −0.037 | 0.423 | 0.435 | 0.632 | 2.03 |
| RAD52 | 693390II [symbol] | −0.0075 | 0.3645 | −1.472 | −1.553 | 0.5215 | 1.179 | −0.6835 | −1.375 | −0.43 | −0.7325 | −2.099 | 0.1365 | −0.4765 | 0.1805 | −1.433 |
| RAD54L | 690991II [symbol] | −0.386 | 0.74 | 0.38 | −0.62 | 0.209 | −0.006 | 1.913 | 1.073 | −0.57 | 1.025 | −0.383 | 0.88 | 0.327 | −0.734 | 0.782 |
| RAMP | 696334II [symbol] | −1.61 | −1.414 | −1.34 | −1.68 | −2.214 | −1.962 | −1.25 | −0.28 | −1.19 | −1.63 | −2.405 | −1.038 | −1.597 | −1.1936 | −1.15 |
| RANBP1 | 698056II [symbol] | −1.55 | −1.204 | −1.362 | −1.896 | −1.13 | −1.298 | −0.835 | −1.39 | −0.06 | 0.264 | −2.144 | −1.292 | −2.048 | −1.658 | −1.049 |
| RARRES [symbol] | 696860II [symbol] | 3.931 | 2.463 | 0.945 | 0.911 | 1.403 | 0.412 | 1.164 | 4.214 | −0.07 | 1.441 | 1.279 | 2.105 | 1.922 | 4.517 | 5.798 |
| RBBP7 | 695677II [symbol] | −0.376 | −0.783 | −0.635 | −0.691 | −0.455 | −0.489 | −0.387 | −0.453 | 0.02 | 0.067 | −0.379 | −1.211 | −0.159 | −1.226 | −0.514 |
| RBBP8 | 690099II [symbol] | −0.086 | 0.343 | −0.164 | 0.348 | −0.585 | −0.145 | −1.033 | −0.331 | 1.35 | 1.389 | 0.485 | −1.16 | 0.903 | −0.171 | −0.391 |
| RBM8A | 681239II [symbol] | −0.3105 | −0.0745 | 0.3765 | −0.1765 | −0.9445 | −0.4575 | 0.9495 | 0.0305 | 0.31 | 0.5535 | −0.9035 | 0.4265 | 0.6225 | −0.8195 | 0.6705 |
| RBX1 | 685240II [symbol] | 0.015 | 0.369 | −0.777 | −0.176 | 0.034 | 0.496 | 0.675 | 0.741 | −0.04 | 0.904 | 0.07 | 0.08 | 0.594 | 0.095 | 0.181 |
| RDHE2 | 695028II [symbol] | 0.68 | −0.109 | 1.61 | 0.968 | 0.235 | −0.174 | 1.021 | 1.226 | 0.31 | 1.206 | −0.187 | 1.553 | 0.201 | 0.295 | 0.666 |
| REA | 691904II [symbol] | −0.023 | 0.15 | 2.024 | −0.101 | 0.04 | −0.054 | −0.006 | 0.234 | −0.04 | 0.757 | −0.053 | −0.268 | 0.465 | 0.724 | 1.315 |
| REC14 | 685901II [symbol] | 0.0005 | 0.5375 | 0.0595 | 0.1775 | −0.3485 | 0.3085 | −0.6255 | −0.5395 | 0.39 | 0.4635 | 0.4745 | −0.8425 | −0.1355 | 0.0945 | −0.4075 |
| REG1A | 695196II [symbol] | 0.479 | −0.607 | −0.002 | 0.18 | −0.508 | 0.047 | 1.236 | 0.375 | 0.08 | 0.263 | 0.135 | 0.266 | 0.326 | 0.171 | 2.386 |
| RELB | 684812II [symbol] | −0.021 | −0.623 | 1.924 | −0.485 | 1.138 | 0.153 | 0.793 | 1.682 | −0.32 | −0.72 | 0.076 | −0.167 | −0.706 | −0.906 | 1.501 |
| REPIN1 | 683066II [symbol] | −0.4075 | 0.3965 | −0.2905 | 0.4605 | 0.3045 | 0.5105 | 0.3105 | −0.5725 | 0.01 | −1.657 | 0.4535 | −0.4035 | −0.1745 | 0.1855 | −0.4345 |
| REPS2 | 687793II [symbol] | 0.5422 | −0.2178 | 2.194 | −1.147 | −0.5378 | 0.4132 | 1.859 | 1.989 | −0.32 | 2.345 | −0.4648 | 1.253 | 0.4612 | 0.1402 | 2.357 |
| RERG | 687590II [symbol] | −0.8165 | 1.61 | −1.088 | 1.092 | 2.118 | −0.1005 | −1.032 | −1.172 | 0.76 | −0.8225 | 0.7675 | −0.1925 | −0.0045 | −0.2185 | −0.1815 |
| RFC4 | 684079II [symbol] | −0.3105 | 0.0225 | 0.6185 | −0.4005 | −0.2875 | −0.3595 | 1.296 | 1.692 | −0.35 | 1.064 | −0.1135 | 0.1715 | −0.0235 | 0.3455 | 1.724 |
| RGS10 | 695917II [symbol] | 1.288 | 0.864 | 1.413 | 1.697 | 0.894 | 0.494 | 2.036 | 2.422 | −0.31 | −0.386 | 0.736 | 3.716 | −0.008 | 1.537 | 1.888 |
| RHBDL2 | 684395II [symbol] | 0.189 | 0.587 | 0.636 | 0.167 | −0.197 | −0.399 | 1.65 | 1.716 | −0.37 | −0.037 | −0.624 | 1.555 | −0.213 | −0.496 | 1.22 |
| RIBC2 | 695320II [symbol] | 0.932 | 0.794 | −0.755 | 0.527 | 0.565 | −0.189 | 1.104 | 1.979 | −0.23 | −0.863 | −0.132 | 0.062 | 0.129 | 0.412 | 0.284 |
| RIC-8 | 690624II [symbol] | −0.91 | −0.279 | −0.06 | −0.767 | 0.096 | −0.716 | −0.443 | 0.245 | 0.35 | 0.373 | −1.154 | −0.551 | −1.317 | −0.098 | 0.784 |
| RIT1 | 693292II [symbol] | −0.214 | −1.003 | −0.519 | −0.715 | −1.87 | −0.711 | −0.147 | 0.974 | 0.23 | −0.105 | −0.716 | 0.987 | 0.678 | 0.078 | 0.109 |
| RLN2 | 688741II [symbol] | 0.233 | −1.677 | 0.613 | −0.682 | 3.3 | 3.554 | −0.379 | −0.847 | 1.14 | −0.95 | 1.022 | −0.498 | −0.164 | 1.366 | −0.976 |
| RNF10 | 696512II [symbol] | 0.564 | 0.536 | −0.431 | 0.607 | 0.62 | 0.257 | 0.062 | 0.279 | 0 | 0.018 | 0.245 | 0.61 | 0.243 | −0.024 | −0.084 |
| RNF135 | 691324II [symbol] | 0.549 | 0.065 | 2.63 | −0.774 | −0.016 | 0.235 | −0.291 | 0.813 | 0.53 | −0.32 | −0.474 | 1.164 | 0.364 | 0.157 | 0.888 |
| RNF7 | 691075II [symbol] | 0.0058 | 1.086 | −0.6673 | −0.4283 | 0.2308 | 0.6398 | −0.7112 | −0.1963 | 0.37 | 0.0688 | 0.5397 | −0.1103 | −0.6623 | 0.0198 | 0.2528 |
| ROPN1 | 692734II [symbol] | −0.798 | −2.361 | −0.103 | −0.927 | −1.895 | −0.584 | 2.759 | 2.609 | 1.28 | −1.398 | −1.367 | 2.807 | −1.272 | −0.4048 | 2.728 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| RPA3 | 684912II [symbol] | 0.119 | −0.497 | 0 | −0.02 | 0.01 | 0.087 | 0.417 | 0.0917 | −0.63 | 0.053 | −0.108 | 0.32 | 0.023 | −0.087 | −0.049 |
| RPESP | 690228II [symbol] | −1.337 | 0.1057 | 1.524 | 0 | 3.455 | −0.518 | −1.286 | −0.909 | 2.89 | −1.612 | −0.311 | −0.461 | −1.065 | 4.244 | −0.763 |
| RPL11 | 684149II [symbol] | 1.035 | 0.337 | 0.42 | 0.424 | 0.379 | 0.667 | 0.609 | 0.127 | 0.56 | 0.285 | 0.856 | 0.788 | 0.137 | 0.144 | 1.298 |
| RPL13 | 690460II [symbol] | −0.3855 | −0.0545 | 0.7695 | −0.4025 | 0.2715 | 0.1305 | −0.0735 | 0.4495 | 0.76 | 0.4475 | −0.2605 | 0.2845 | −0.4925 | 0.2335 | 0.3065 |
| RPL13A | 697951II [symbol] | −0.695 | −0.465 | 1.329 | 0.38 | −0.12 | −0.104 | −0.401 | −0.286 | 0.14 | −0.096 | −0.02 | −1.208 | 1.61 | 0.255 | −0.331 |
| RPL15 | 682985II [symbol] | 0.4203 | 0.5413 | 0.2203 | −0.4397 | −0.4147 | −0.0458 | 1.81 | 1.31 | 0.85 | 0.9703 | −0.0007 | 0.9493 | 0.2593 | 0.5443 | 0.8503 |
| RPL18A | 683895II [symbol] | 0.3535 | −0.7485 | 1.063 | 0.3045 | −0.0195 | −0.2205 | −0.1645 | −0.4535 | −0.13 | 0.8535 | −0.0825 | −1.019 | 0.0565 | 0.9205 | 0.4045 |
| RPL19 | 688555II [symbol] | 0.422 | −0.421 | −0.382 | 0.006 | 0.7 | −0.382 | −0.465 | 0.422 | 0.14 | −0.584 | −0.052 | 0.192 | −0.257 | −0.447 | 0.063 |
| RPL21 | 692099II [symbol] | 0.0895 | −1.342 | 0.6665 | 0.2065 | −0.9965 | −0.0065 | 1.025 | 0.5985 | 0.44 | 0.2215 | −0.1945 | −0.3975 | 0.1125 | −0.1305 | 0.7445 |
| RPL23A | 681568II [symbol] | −0.578 | −0.496 | 0.986 | 0.017 | 0.195 | −0.432 | −0.525 | 1.05 | 0.23 | 0.032 | −0.035 | −0.477 | −0.381 | 0.056 | −0.017 |
| RPL23A | 692544II [symbol] | −0.354 | −0.273 | −0.921 | −0.468 | −0.548 | −0.369 | −0.201 | 0.235 | 0.25 | −0.508 | −0.36 | −0.503 | −1.006 | 0.272 | −0.059 |
| RPL26L1 | 685938II [symbol] | −0.044 | −0.195 | 0.474 | −0.359 | −0.254 | −0.174 | 1.314 | 0.878 | 0.45 | 1.603 | −0.779 | 0.294 | 0.542 | 0.416 | 1.056 |
| RPL29 | 690810II [symbol] | −0.272 | 0.412 | 0.666 | 0.27 | 1.005 | −0.043 | −0.758 | −0.386 | 0.29 | 0.596 | −0.419 | −0.61 | −0.435 | 0.495 | −0.445 |
| RPL36 | 687324II [symbol] | −0.021 | −1.157 | 0.968 | 0.303 | 0.687 | −0.277 | −0.168 | −0.557 | 0.3 | −0.432 | 0.007 | −0.462 | −0.359 | 0.245 | 0.005 |
| RPL36AL | 693407II [symbol] | 0.07 | 0.61 | −3.914 | −0.528 | −4.797 | −1.496 | −4.564 | −3.785 | 0.09 | −3.546 | 0.317 | −3.929 | 0.643 | −3.825 | −1.758 |
| RPS16 | 687942II [symbol] | −0.6585 | 0.0245 | 1.617 | 0.2295 | −0.4415 | −0.4365 | 0.1185 | 0.4505 | −0.09 | 0.3665 | −0.6145 | −1.314 | 0.0175 | 0.5765 | −0.1425 |
| RPS4Y1 | 683806II [symbol] | 0.318 | 0 | 1.062 | −0.393 | −0.277 | −0.638 | 0.117 | −0.119 | 0.48 | 1.173 | −0.211 | −0.343 | 0.065 | −0.212 | 0.827 |
| RRAGA | 688744II [symbol] | −0.797 | −0.018 | −0.758 | 0.228 | 0.373 | 0.341 | −0.119 | −0.436 | 0.41 | −0.03 | 0.609 | −0.231 | −0.366 | 0.019 | 0.255 |
| RRAGD | 691801II [symbol] | 0.6827 | −1.098 | 2.148 | 0.1787 | −0.1353 | −0.3183 | −0.1283 | 1.774 | −0.81 | 0.0788 | −0.2743 | 0.5957 | 0.6057 | −0.2653 | 0.3907 |
| RRM1 | 684258II [symbol] | 0.1005 | −0.1195 | 0.5305 | 0.2015 | −0.0305 | 0.2205 | 0.1315 | 0.6475 | 0.02 | 0.6355 | 0.4045 | −0.4015 | −0.5015 | −0.2305 | 0.6145 |
| RRM2 | 694262II [symbol] | −0.756 | −1.513 | −1.914 | −3.078 | −1.924 | −4.008 | −1.355 | −1.322 | −1.15 | 0.52 | −2.216 | −3.059 | −1.729 | −2.333 | −1.767 |
| RSAFD1 | 687475II [symbol] | −0.3945 | −0.3015 | 0.4665 | 0.2375 | 0.0485 | 1.402 | −0.3725 | 0.0325 | 0.27 | −0.1245 | −0.6825 | −0.8295 | 0.1095 | −0.1725 | −0.3265 |
| RSNL2 | 692893II [symbol] | −0.844 | −1.451 | −0.262 | −1.773 | −0.745 | −0.835 | −0.352 | −0.003 | 0.84 | −2.078 | 0.535 | −0.3 | 0.435 | −1.079 | 1.874 |
| RSU1 | 682633II [symbol] | −0.0025 | 0.2105 | 0.3425 | −0.6875 | −0.6765 | −0.3785 | 1.041 | 1.171 | −0.23 | −0.1975 | 0.2325 | 0.4285 | 0.8035 | −0.1075 | 0.8445 |
| RTN4IP1 | 696006II [symbol] | −1.125 | −0.71 | −1.142 | −0.692 | −0.875 | −0.971 | −1.003 | −0.105 | −0.45 | −0.183 | −0.864 | −0.298 | −0.327 | −1.251 | −0.013 |
| S100A11 | 688967II [symbol] | −0.043 | 0.206 | −0.439 | −0.559 | −1.212 | −0.173 | 0.227 | −0.104 | −0.11 | 0.009 | −0.212 | 0.751 | 0.343 | 0.356 | 0.272 |
| S100A14 | 689448II [symbol] | 1.787 | 0.7805 | 1.313 | −0.7455 | 0.0765 | 0.8185 | −1.195 | −0.0765 | −0.16 | −3.893 | −0.1915 | −0.4735 | 2.072 | 0.9225 | 0.6795 |
| S100A16 | 682683II [symbol] | 1.547 | 1.001 | −0.246 | −1.06 | 0.02 | −0.649 | −1.683 | −1.584 | 0.21 | −1.815 | 0.238 | −0.317 | 1.662 | 0.44 | 0.849 |
| S100A9 | 681875II [symbol] | −1.751 | −0.1485 | −1.949 | −2.16 | 0.192 | −1.265 | 0.577 | −0.537 | 0.2 | −2.267 | 0.872 | 0.032 | −0.413 | 0.822 | 0.731 |
| SAE1 | 695822II [symbol] | −1.344 | −0.221 | −0.792 | −0.762 | −0.787 | −0.51 | −0.112 | −0.738 | −0.13 | −0.083 | −0.752 | −1.722 | −1.196 | −1.183 | −0.503 |
| SALL2 | 696484II [symbol] | −1.648 | −0.187 | −0.715 | −0.043 | −1.453 | −0.128 | −1.134 | −1.93 | 1.19 | −1.177 | −0.337 | −0.714 | 0.572 | −1.36 | −1.327 |
| SARA1 | 690066II [symbol] | 0.364 | 0.156 | −0.005 | −0.357 | −0.329 | 0 | −0.057 | 1.369 | −0.54 | 0.935 | 0.107 | 0.056 | −0.32 | −0.375 | −0.06 |
| SARA2 | 688945II [symbol] | 0.144 | 0.632 | −0.699 | 0.091 | 0.182 | 0.068 | −0.519 | −0.348 | −0.51 | 0.303 | 0.537 | −0.505 | 0.081 | −0.127 | 0.177 |
| SAS | 697574II [symbol] | 2.364 | −1.065 | −1.765 | −0.934 | 0.236 | −1.989 | 0.651 | −2.133 | 0.54 | −1.703 | 0.32 | 0.873 | 0.43 | 0.587 | 1.652 |
| SCIN | 698329II [symbol] | 1.468 | 1.0052 | 0.332 | 0.995 | 0.341 | 2.402 | 1.044 | −0.055 | −0.69 | 0.54 | 1.357 | 1.02 | 0.286 | 0.716 | 0.65 |
| SCML1 | 683929II [symbol] | 0.4585 | −0.2995 | −0.6135 | −1.262 | 2.061 | 1.104 | −0.3255 | −1.387 | 0.38 | −0.7785 | 0.7995 | −1.384 | −0.8075 | −1.402 | 0.9905 |
| SCPEP1 | 689318II [symbol] | 0.0718 | −0.8382 | 0.1308 | −0.7552 | −0.6022 | −0.0763 | −0.0253 | 1.678 | 1.07 | −0.9152 | 0.2508 | 1.703 | −0.6332 | 0.3658 | 0.6858 |
| SCRIB | 687588II [symbol] | −0.1103 | 1.02 | 0.4267 | 0.0548 | 0.0128 | 0.7667 | 1.336 | 1.233 | −0.23 | −0.5613 | −0.3343 | 1.324 | −0.0993 | −1.118 | −0.1373 |
| SDC1 | 681259II [symbol] | 1.56 | 1.103 | −0.5535 | 0.3235 | −0.9955 | 0.7675 | 0.9165 | −0.0985 | 0.01 | −1.111 | −0.0945 | 1.088 | −0.1805 | 1.037 | −0.3715 |
| SDC3 | 693165II [symbol] | 1.442 | −1.521 | 0.238 | −0.024 | −1.087 | 1.14 | 0.732 | 0.081 | 0.93 | 0.234 | 1.412 | 1.54 | 1.2 | −0.221 | 0.54 |
| SDCCAG [symbol] | 690950II [symbol] | 0.059 | 0.04 | 0.674 | −0.01 | 2.689 | −0.746 | 0.132 | 0.895 | 0.15 | −0.77 | −0.597 | 0.652 | 0.241 | −0.223 | 0.354 |
| SEC61G | 693650II [symbol] | −0.298 | −0.978 | −1.537 | −1.002 | −0.341 | −1.528 | −0.592 | −1.138 | −0.78 | 0.511 | −1.123 | −1.199 | 0.27 | −0.702 | −0.324 |
| SECP43 | 694767II [symbol] | 0.324 | 0.208 | −0.734 | −0.294 | −0.267 | 0.29 | 0.598 | −0.308 | 0.04 | −0.626 | −0.303 | 0.621 | 0.462 | −0.844 | 0.05 |
| SEDLP | 693304II [symbol] | −0.024 | −0.053 | 0.424 | 0.943 | −0.172 | −1.013 | −0.321 | 0.261 | −0.07 | 0.707 | −0.07 | −1.019 | −0.816 | 0.198 | −0.321 |
| SEMA3B | 696622II [symbol] | −1.244 | −1.236 | −1.507 | −0.277 | −0.794 | 0.497 | −0.68 | −2.055 | 1.39 | −2.146 | −0.046 | −1.671 | 1.37 | −0.278 | −2.442 |
| SEMA3C | 691613II [symbol] | 0.8405 | 1.047 | −3.177 | 0.9065 | 1.582 | 1.416 | −1.843 | −2.957 | 1.87 | −2.605 | 0.9655 | −2.527 | −0.6545 | −0.6475 | −1.387 |
| SEPW1 | 683289II [symbol] | −0.896 | 0.35 | 0.54 | 0.499 | −0.17 | 0.372 | 0.579 | −0.014 | −0.39 | −0.308 | 0.447 | −0.146 | 0.547 | −0.434 | −0.135 |
| SERP1 | 690913II [symbol] | 0.0363 | 0.0073 | −0.3207 | 0.5823 | 1.188 | −0.7667 | −1.286 | 0.0063 | −0.29 | −0.7557 | −0.2087 | −1.267 | −1.554 | 0.5833 | 1.136 |
| SERPINA [symbol] | 692607II [symbol] | −1.627 | −0.911 | −2.651 | −1.558 | −1.982 | 0.536 | −2.486 | −2.367 | 0.59 | −2.305 | −0.244 | −1.212 | −0.513 | −2.179 | −2.87 |
| SERPINB [symbol] | 683845II [symbol] | 1.293 | −0.1835 | 1.858 | −0.1985 | 0.2105 | 0.0165 | 1.453 | 1.215 | 0.79 | −0.7535 | 3.435 | 2.083 | 1.274 | 0.1025 | 1.959 |
| SFRP1 | 690138II [symbol] | −1.172 | −0.601 | 2.765 | 0.115 | −0.42 | −0.616 | 0.014 | 3.29 | 2.9 | 2.569 | −0.209 | 5.578 | −0.525 | −0.254 | 1.833 |
| SFRS6 | 689301II [symbol] | 0.3545 | 0.2365 | 0.0465 | 0.2425 | 0.0775 | −0.0735 | −0.7005 | −0.7535 | −0.71 | 1.001 | 1.136 | −0.5155 | −0.3725 | 0.1135 | −0.8015 |
| SGCD | 694761II [symbol] | −0.54 | 0.106 | −1.263 | 0.053 | −0.835 | −0.417 | −1.587 | −0.424 | −0.27 | −0.363 | 0.576 | −0.466 | 0.384 | −0.576 | −1.047 |
| SGCE | 685105II [symbol] | −0.645 | 0.423 | 1.175 | −1.105 | −0.743 | −0.173 | 0.135 | 1.675 | 1.61 | 0.225 | −0.059 | 0.109 | −0.482 | −0.401 | 0.742 |
| SH3BGRL | 694455II [symbol] | 0.541 | 0.633 | 0.821 | 3.353 | −0.201 | 1.381 | −0.736 | 0.625 | 0.77 | −1.556 | 1.748 | −0.634 | 2.064 | 0.771 | 0.688 |
| SHFM1 | 683462II [symbol] | 0.0705 | 0.1785 | 1.18 | −0.0525 | 0.0135 | −0.1465 | 1.57 | 1.545 | −0.34 | 0.5115 | −0.3895 | 0.3635 | 0.0745 | −0.1625 | 0.3745 |
| SIAT7B | 695595II [symbol] | 0.474 | 0.648 | −1.386 | −0.085 | −0.891 | −0.23 | 0.107 | −0.456 | 0.95 | −0.674 | 0.561 | −0.336 | 1.478 | −1.29 | −1.192 |
| SIGLEC12 | 690532II [symbol] | 0.9615 | 0.3165 | 0.6715 | 0.0615 | 0.5655 | −0.1595 | −0.0035 | −0.0995 | 0 | −0.2385 | −0.0505 | 0.1095 | −0.1665 | 0.0035 | 0.3385 |
| SIT | 692124II [symbol] | 1.221 | −0.336 | −1.362 | −0.306 | 0.673 | −0.132 | 1.406 | −0.946 | −0.26 | −1.108 | 0.477 | −0.163 | 0.034 | −0.334 | 1.191 |
| SITPEC | 681337II [symbol] | −0.0235 | −0.3815 | −0.1555 | 0.6555 | 0.1025 | 0.2105 | −0.3265 | −0.6655 | 0.09 | 0.1475 | −0.0945 | −0.6465 | −0.5895 | −0.5095 | −0.8945 |
| SKP1A | 684965II [symbol] | −0.764 | −0.829 | −0.799 | −0.005 | −0.964 | 0.104 | −0.849 | −1.075 | 0.09 | 0.23 | −0.108 | −1.407 | 0.779 | 0.005 | 0.268 |
| SLC12A9 | 696704II [symbol] | 0.353 | 0.21 | 0.531 | 0.672 | 0.845 | 0.674 | 1.188 | 0.672 | 0 | 0.281 | 0.384 | 0.509 | 0.166 | 0.423 | 0.093 |
| SLC13A3 | 689746II [symbol] | 0.5155 | −0.6385 | 1.707 | −1.379 | −0.3225 | 0.2415 | 0.0935 | 0.3255 | −0.18 | 1.579 | −1.039 | 2.051 | −0.9745 | 0.3765 | 0.7935 |
| SLC16A6 | 687056II [symbol] | −0.0835 | −0.8115 | −1.98 | 2.134 | 1.964 | 1.646 | −1.324 | −1.763 | 1.48 | 0.4175 | 0.5865 | −2.304 | −0.3345 | −1.072 | −1.501 |
| SLC19A2 | 693146II [symbol] | −0.932 | 1.729 | 0.36 | 0.834 | 0.277 | −0.446 | −1.218 | 0.11 | 0.5 | −1.398 | 1.311 | −0.13 | 0.19 | −0.956 | −0.789 |
| SLC1A1 | 692391II [symbol] | 0.889 | 0.618 | 1.217 | 0.552 | 1.182 | −0.119 | 1.038 | 0.533 | 3.29 | 1.802 | 0.887 | 0.764 | 0.906 | 0.558 | −0.17 |
| SLC1A2 | 690726II [symbol] | −0.9725 | 1.233 | 0.3015 | 2.282 | 1.061 | 1.712 | −1.669 | 2.009 | −0.41 | −1.592 | 1.658 | −0.3585 | 0.3365 | −0.6735 | −1.396 |
| SLC24A3 | 689570II [symbol] | −0.5655 | −0.0265 | 0.7215 | −2.329 | −2.29 | −0.8075 | −0.3035 | −0.4865 | 0.05 | −1.625 | 0.9115 | −0.2575 | −0.8735 | 0.0265 | 0.9865 |
| SLC25A2 [symbol] | 692129II [symbol] | 1.193 | 0.146 | 0.91 | 0.131 | −0.034 | 0.477 | −0.346 | −0.428 | 0.01 | −0.496 | 0.229 | −0.423 | 0.682 | 0.763 | −0.602 |
| SLC25A4 | 684980II [symbol] | −0.9085 | 0.7055 | −0.4965 | 0.9845 | 0.9845 | −0.4375 | 0.4675 | 0.1795 | 0.06 | −0.1805 | 0.3775 | −0.1165 | 0.0145 | −1.118 | −0.0145 |
| SLC25A5 | 681570II [symbol] | 0.024 | 0.321 | 1.174 | −0.374 | −0.71 | −0.403 | 0.563 | 1.199 | −0.29 | 0.255 | −0.266 | −1.08 | 0.144 | 1.026 | 0.768 |
| SLC27A2 | 686936II [symbol] | 0.2693 | −0.2677 | −1.345 | −1.045 | 4.006 | 2.719 | −1.052 | −1.38 | 1.29 | −1.8 | 1.892 | −1.487 | 1.202 | −0.5087 | −0.2567 |
| SLC27A5 | 697266II [symbol] | −1.421 | −0.614 | −0.206 | −1.197 | 0.181 | −0.927 | −0.081 | 0.029 | 0.05 | −1.788 | −1.67 | −1.289 | −0.325 | 0.073 | −0.768 |
| SLC30A8 | 695934II [symbol] | 0.1566 | 1.139 | 0.971 | 2.881 | 2.126 | 0.313 | −0.084 | −0.099 | 0.07 | −0.058 | 0.11 | 0.606 | 0.092 | 0.27 | −0.259 |
| SLC39A4 | 684775II [symbol] | −0.722 | 0.43 | −0.426 | −0.75 | 0.326 | 0.27 | 1.234 | 1.3 | −0.24 | 0.745 | −1.143 | 1.858 | 0.112 | −0.294 | −0.313 |
| SLC39A6 | 690413II [symbol] | −2.088 | 0.5945 | −0.6945 | 2.988 | 3.546 | 1.403 | −2.594 | −1.108 | 2.21 | −2.438 | 2.089 | −1.515 | −1.288 | −1.271 | −1.232 |
| SLC40A1 | 695661II [symbol] | 2.251 | 1.547 | 0.898 | 1.814 | 2.976 | 3.28 | 1.292 | −1.913 | 1.68 | 1.178 | 3.828 | −0.216 | 1.333 | 3.713 | 0.898 |
| SLC41A3 | 682343II [symbol] | −0.3485 | −0.0745 | 0.2065 | 0.0335 | 0.4095 | 0.2565 | −0.0045 | 0.1715 | 0.43 | 0.2955 | −0.0285 | 0.6995 | −0.8705 | 0.0045 | 0.5035 |
| SLC5A6 | 688366II [symbol] | 0.147 | 0.083 | 1.773 | −1.152 | −0.784 | 0.733 | 0.214 | 1.508 | −0.54 | 1.586 | −0.711 | −0.003 | 0.197 | 1.323 | 1.277 |
| SLC6A4 | 691977II [symbol] | −0.026 | −1.383 | 0.302 | 0.873 | 3.557 | 0.733 | −0.207 | −0.075 | 1.23 | 0.381 | 2.51 | −0.093 | −0.063 | 0.085 | −0.12 |
| SLC9A3R [symbol] | 682747II [symbol] | 0.825 | 2.043 | −2.176 | 0.766 | 0.952 | −0.474 | −1.047 | 0.016 | −0.22 | −0.908 | 1.505 | 0.007 | 0.823 | 0.04 | −0.756 |
| SLITRK6 | 685643II [symbol] | 0.521 | 0.036 | 0.423 | −0.401 | −1.307 | −1.038 | 1.448 | 1.759 | 3.07 | 1.32 | −0.412 | 0.579 | 0.447 | −0.036 | 0.696 |
| SMA3 | 687200II [symbol] | 2.007 | 1.115 | −1.021 | 1.819 | 1.215 | 0.0025 | −1.056 | −0.9755 | 0.83 | −1.925 | 1.172 | −1.917 | 1.784 | 0.1516 | −1.396 |
| SMAD2 | 695457II [symbol] | −0.321 | −0.496 | 0.888 | −0.631 | −0.414 | −0.183 | 0.167 | 0.209 | 0.19 | −0.577 | −0.417 | 0.119 | −0.155 | 0.056 | 0.511 |
| SMO | 686958II [symbol] | −0.43 | −0.791 | 1.318 | −0.78 | 0.176 | −0.137 | 0.624 | 0.995 | 0.27 | 0.030 | 0.14 | 2.065 | −0.635 | 0.234 | −0.068 |
| SMU1 | 689287II [symbol] | −0.18 | 0.228 | −0.038 | 0.343 | 0.255 | −0.503 | 0.283 | 0.355 | −0.11 | 1.17 | 0.038 | −0.31 | −0.395 | −0.534 | −0.172 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| SNPH | 684407II [symbol] | 2.091 | −0.6652 | 0.1028 | 0.7698 | 0.4908 | −0.2252 | 0.0448 | −0.0613 | −0.07 | −0.3902 | −0.0493 | −0.0353 | −0.2052 | −0.2372 | −0.3822 |
| SNRPB2 | 696839II [symbol] | −1.303 | 0.149 | −0.107 | −1.291 | −1.063 | −1.042 | −0.172 | −0.513 | −0.29 | 0.426 | −1.072 | −0.562 | −0.531 | −0.755 | −0.495 |
| SNRPD1 | 695576II [symbol] | −1.632 | −1.906 | 0.041 | −2.01 | −1.445 | −1.467 | −0.214 | 0.063 | −0.13 | 0.005 | −2.262 | −0.957 | −1.224 | −1.252 | −0.515 |
| SNRPD3 | 692082II [symbol] | 0.4215 | 0.0955 | −0.2545 | −0.0095 | 0.0365 | −0.1365 | 0.2975 | 0.2565 | −0.15 | 0.6845 | −0.0645 | 0.1655 | −0.3545 | −0.0725 | 0.2625 |
| SNRPG | 684507II [symbol] | −0.001 | −0.286 | 0.725 | −0.238 | −0.68 | −0.295 | 0.161 | 1.027 | −0.26 | 0.708 | −0.503 | 0.463 | 0.13 | −0.155 | 0.404 |
| SNX7 | 685968II [symbol] | −1.289 | −0.0245 | −0.5705 | −0.4295 | 0.4865 | −0.4615 | 0.8235 | 1.276 | 0.62 | −0.7685 | 0.0855 | −0.4525 | −1.444 | −1.671 | 0.8145 |
| SOD2 | 684100II [symbol] | 1.273 | −1.085 | 0.1563 | −0.6177 | 0.3783 | −0.3797 | −0.5267 | 3.133 | −0.84 | 1.09 | 0.4003 | 1.377 | 0.7443 | 0.6523 | 1.881 |
| SORCS1 | 690320II [symbol] | −0.3215 | 0.2215 | −0.3555 | 0.7895 | −1.142 | −0.3395 | −0.2555 | −0.0255 | 0.36 | −0.7135 | 0.7465 | 0.0695 | 0.4395 | −0.0285 | 0.1235 |
| SOX4 | 686301II [symbol] | −0.0815 | 1.005 | 0.2225 | −0.2455 | −1.799 | 0.7005 | −0.3595 | −0.0005 | −0.2 | −1.026 | −0.2305 | 0.5325 | 0.3615 | −0.5585 | −0.4725 |
| SOX9 | 681418II [symbol] | 1.355 | 1.846 | 2.21 | 1.248 | 1.149 | 0.571 | 0.037 | 0.336 | 0.49 | 2.587 | 1.136 | 0.651 | −0.152 | 0.242 | 0.552 |
| SPAG16 | 687462II [symbol] | 0.8158 | 2.1 | 0.9868 | −0.7202 | −0.3052 | 0.0468 | 0.4938 | 0.5408 | 0.26 | −0.0363 | −0.0903 | −0.1042 | 0.4498 | 0.1588 | 0.5018 |
| SPAG5 | 686120II [symbol] | 0.7525 | 0.6155 | 1.334 | −0.1415 | −0.0375 | −0.7725 | 1.621 | 2.169 | −0.97 | 1.167 | −0.3895 | 1.06 | 0.7835 | 0.0345 | 1.023 |
| SPC18 | 695899II [symbol] | −1.381 | −3.19 | −1.213 | −1.497 | −2.878 | −1.841 | −1.486 | −1.38 | 0.25 | 0.894 | −1.852 | −1.703 | −0.65 | −0.816 | −1.069 |
| SPFH2 | 686593II [symbol] | 0 | 0.3462 | 0.372 | 1.336 | −0.18 | 1.381 | −0.625 | −0.589 | −0.04 | 1.088 | −0.087 | 0.796 | 0.461 | −0.455 | −0.709 |
| SPG3A | 685052II [symbol] | −0.501 | −0.043 | −0.098 | 1.334 | 0.49 | 0.252 | −0.737 | −0.115 | 0.45 | −1.122 | 0.821 | −0.418 | −0.181 | −0.086 | −0.275 |
| SPPL3 | 684761II [symbol] | −0.1785 | 0.1175 | −4.237 | −0.3695 | 0.1155 | 0.7135 | 0.3165 | 0.7985 | 0.12 | −0.1155 | −0.0295 | 0.6885 | −0.5425 | −1.058 | 0.3455 |
| SQLE | 765400II [symbol] | −1.538 | 0.14 | −0.481 | −1.974 | −1.54 | −1.34 | 0.079 | −0.189 | −0.6 | −0.488 | −1.5 | −0.35 | −0.84 | 0.15 | 0.4 |
| SQRDL | 693902II [symbol] | 0.4455 | 0.1895 | −0.7415 | 0.4205 | 0.7915 | 0.3405 | −0.0865 | 0.2055 | −0.15 | −0.5655 | −0.5915 | −0.5405 | −1.111 | −0.4075 | −0.4355 |
| SREBF1 | 697880II [symbol] | 1.373 | 1.997 | −2.013 | 3.289 | 2.38 | 2.389 | 0.08 | 0.84 | 0.78 | −0.776 | 1.425 | −0.294 | 0.982 | 1.245 | 0.093 |
| SRI | 698971II [symbol] | 0.184 | 0.551 | 0.18 | −0.298 | 0.503 | −0.587 | 1.275 | 4.243 | 0.34 | 0.29 | −0.592 | 1.601 | 0.011 | 0.253 | −0.109 |
| SRPK1 | 697017II [symbol] | −1.091 | −2.202 | −0.142 | −1.729 | −0.593 | −1.363 | −0.77 | 0.422 | −0.06 | −1.093 | −1.402 | −0.408 | −0.805 | −0.186 | 0.124 |
| SSFA2 | 692106II [symbol] | −0.3641 | −0.51 | −1.466 | 1.2 | 0.282 | −0.478 | −1.57 | −1.153 | 0.4 | −1.41 | 0.706 | −1.281 | −1.149 | −0.504 | −0.426 |
| SSR4 | 686717II [symbol] | 1.209 | 1.703 | 1.092 | 2.517 | 2.17 | 2.385 | 2.135 | −0.219 | −0.37 | 0.875 | 2.192 | 1.659 | 1.546 | 1.872 | 1.891 |
| STARD3N | 686038II [symbol] | 0.184 | −0.38 | −0.202 | 0.527 | 0.139 | −0.114 | 0.008 | −0.605 | −0.2 | 0.623 | 0.499 | −0.611 | 0.671 | 0.36 | 0.476 |
| STARD7 | 681306II [symbol] | −0.4835 | −0.0225 | 0.5145 | −0.5745 | −0.2065 | −0.0215 | 0.4105 | 0.6885 | −0.23 | 0.0215 | −0.3045 | −0.2005 | −0.8275 | −0.0505 | 0.3175 |
| STAT1 | 681492II [symbol] | −0.0425 | 0.3845 | −0.3725 | −1.139 | 1.567 | −1.193 | 0.1205 | −2.17 | −0.79 | −1.221 | 1.622 | −0.7515 | 0.8105 | 0.8945 | 1.947 |
| STC2 | 689625II [symbol] | −0.7195 | 0.7165 | −1.526 | −0.9935 | −0.5765 | 0.8595 | −0.2245 | 0.4695 | 2.41 | 0.3545 | 1.389 | −0.7265 | −0.7285 | 0.1525 | −0.1525 |
| STK24 | 688430II [symbol] | −0.3955 | −0.5455 | 1.384 | 0.3335 | 0.0345 | −0.0805 | −0.8235 | −0.9685 | 0.13 | −1.291 | 0.6275 | −1.703 | −1.033 | 0.1225 | 0.9135 |
| STK6 | 685902II [symbol] | 0.192 | 0.483 | 0.213 | −0.677 | −0.654 | −0.69 | 0.785 | 1.085 | −0.51 | 3.203 | 0.915 | 0.405 | 0.238 | 0.047 | 0.654 |
| STMN1 | 682764II [symbol] | 0.7995 | 0.0715 | 0.7505 | −0.3695 | −0.8905 | −0.1605 | 2.013 | 1.359 | −0.63 | 0.8825 | −0.0395 | 1.347 | 1.583 | −1.007 | 0.6755 |
| STX1A | 691816II [symbol] | 0.88 | −0.306 | 0.05 | 1.444 | −0.186 | 0.162 | 0.077 | 0.349 | 0.08 | −0.569 | −0.506 | −0.159 | 1.132 | 0.983 | −0.48 |
| STX3A | 691051II [symbol] | 0.1595 | 0.2305 | 0.4955 | −0.4395 | −0.1905 | 0.3805 | −0.5855 | −0.1595 | 0.34 | 0.6485 | −0.1575 | −0.0555 | −0.8055 | 0.1935 | −0.0565 |
| STYX | 685620II [symbol] | 0.689 | −0.431 | 0.056 | 0.35 | −0.36 | −1.034 | −0.023 | 0.78 | −0.72 | 2.551 | −0.532 | −0.573 | 0.744 | 1.48 | 0.386 |
| SUGT1 | 693218II [symbol] | −0.118 | 0.103 | 0.073 | 0.086 | 0.404 | −0.902 | −0.351 | 0.139 | −0.17 | 0.294 | −1.133 | −0.417 | −0.48 | −0.912 | −0.561 |
| SUMF1 | 695843II [symbol] | 2.289 | −1.876 | 0.8525 | −0.8285 | −1.864 | −0.9445 | −0.6485 | 3.217 | 0.57 | −1.624 | 1.013 | 5.808 | −0.0825 | 6.322 | 4.108 |
| SUMO2 | 689403II [symbol] | 0.053 | 0.678 | 0.462 | 0.017 | −0.594 | −0.209 | −0.122 | 0.658 | 0.26 | 0.195 | −0.057 | −0.417 | 0.412 | −0.467 | 0.068 |
| SUV39H2 | 681342II [symbol] | −0.17 | 0.073 | 1.671 | −0.768 | 0.001 | −0.745 | 0.323 | 1.623 | −0.3 | 0.916 | −0.366 | 0.402 | −0.156 | −0.077 | 2.447 |
| SYAP1 | 681813II [symbol] | 1.446 | 1.633 | −0.086 | 1.059 | 0.066 | −0.462 | −0.535 | −0.121 | −0.46 | 0.706 | 0.2 | −0.153 | 1.267 | −0.033 | −0.107 |
| SYCP2 | 689491II [symbol] | −0.778 | 0.5517 | 0.452 | −0.007 | −2.309 | 0.655 | 0.431 | 1.291 | 0.3 | −2.224 | 1.494 | −0.432 | −0.796 | 0.814 | −2.801 |
| SYNGR1 | 686385II [symbol] | 0.8505 | 1.447 | −0.6745 | −0.6465 | −1.569 | 0.7675 | −0.5555 | 1.604 | 0.17 | 1.554 | 0.6065 | 1.959 | 2.236 | 0.0485 | −0.1185 |
| SYTL2 | 692920II [symbol] | 0.484 | 2.491 | −2.244 | −0.084 | 1.402 | −0.229 | −2.071 | −1.2 | 1.09 | 3.002 | 0.107 | −1.215 | 0.682 | −1.452 | −3.129 |
| TAF1B | 681153II [symbol] | 0.356 | 0.241 | −0.055 | −0.264 | −0.002 | 0.22 | 0.423 | −0.094 | 0.15 | 1.651 | 0.236 | −0.349 | 0.216 | −0.674 | −0.225 |
| TAF6 | 687144II [symbol] | −0.158 | −0.584 | 0.116 | 0.59 | 0.079 | 0.04 | 1.1 | 1.216 | −0.01 | −0.017 | −0.031 | 1.071 | 0.017 | −0.835 | −0.133 |
| TARBP1 | 697518II [symbol] | −0.475 | 0.05 | 1.78 | 0.548 | −0.132 | −0.128 | 0.547 | 1.28 | 0.69 | −0.724 | 0.065 | 1.305 | 0.441 | 0.113 | 0.586 |
| TARS | 686090II [symbol] | 0.34 | −0.443 | −0.133 | −0.472 | 0.279 | −0.225 | 0.292 | 0.666 | −0.26 | 0.394 | −0.192 | 0.665 | 0.382 | 0.18 | 0.617 |
| TATDN1 | 692620II [symbol] | 0.41 | 0.037 | 0.29 | −0.215 | −0.059 | −1.069 | 0.272 | 1.194 | −0.19 | 1.236 | −0.953 | 0.857 | 0.148 | 0.255 | 0.256 |
| TBC1D12 | 689394II [symbol] | 0.0435 | −0.2975 | 2.585 | −0.1265 | −0.1075 | −0.7165 | 0.0205 | 1.846 | 0.22 | 0.7705 | −0.3785 | 0.1695 | 0.6615 | 0.2705 | 1.823 |
| TBC1D14 | 683294II [symbol] | −0.2092 | −0.4972 | 0.1648 | 0.5928 | 0.4028 | 0.6098 | 0.0138 | −0.5352 | 0.32 | −1.561 | −0.2372 | 0.6968 | 0.1308 | −0.1872 | 0.2258 |
| TBC1D3 | 684780II [symbol] | 0.467 | −1.826 | 0.276 | −0.357 | 0.182 | 0.517 | 0 | 0.407 | 0.4 | −0.605 | −0.576 | −0.233 | 0.43 | 1.861 | 0.478 |
| TBCA | 689449II [symbol] | −0.2675 | −0.6155 | 0.0165 | 0.2405 | 0.0025 | −0.3495 | 0.4105 | −0.1395 | −0.6 | 0.0475 | −0.5305 | −0.6435 | 0.8325 | 0.0865 | 0.0475 |
| TBX1 | 682010II [symbol] | 0.3065 | 2.254 | 0.9025 | 0.5765 | 0.5945 | −0.4725 | −0.8145 | −0.8695 | −0.2 | 0.0925 | −1.201 | 1.464 | 1.241 | 1.542 | −1.576 |
| TBX3 | 687903II [symbol] | −2.203 | 1.318 | −2.884 | 0.907 | 0.651 | 1.434 | −2.146 | −1.933 | 1.58 | −2.825 | 0.807 | −1.371 | 0.645 | −1.719 | −2.104 |
| TCAP | 680995II [symbol] | 0.7675 | 0.5745 | −0.5265 | −0.3405 | −0.1605 | −0.4735 | 0.1995 | 0.5155 | 0.3 | −0.1925 | −0.1145 | 0.2505 | −0.0815 | 0.4065 | 0.2745 |
| TCEAL1 | 688759II [symbol] | −0.7125 | −0.7415 | −0.2205 | 0.5595 | −0.5575 | 0.3545 | −0.3485 | −1.196 | 1.01 | −0.4775 | 1.242 | −0.7755 | 0.2375 | −0.4785 | −0.9255 |
| TCEAL3 | 691252II [symbol] | 0.545 | 4.173 | −0.145 | −0.205 | 0.727 | −0.493 | −0.295 | 0.035 | 1.13 | 0.505 | −0.687 | −0.399 | −0.687 | −0.151 | −0.025 |
| TCEAL4 | 687353II [symbol] | 0.968 | −0.018 | 0.086 | 0.424 | 0.864 | −0.846 | −0.586 | −1.252 | 0.7 | −0.578 | −0.452 | 0.376 | 0.853 | 0.506 | 0.648 |
| TCF15 | 685545II [symbol] | 0.3045 | −0.0795 | 0.5125 | −0.6385 | −0.8025 | 0.5425 | 2.161 | 1.83 | −0.62 | −1.943 | −0.5865 | 0.9125 | 0.5145 | 0.1535 | −0.5055 |
| TCF3 | 689758II [symbol] | 1.645 | −0.022 | 2.365 | −0.72 | −0.273 | 0.376 | 1.398 | 1.26 | −0.05 | 0.248 | −0.458 | 1.212 | 0.246 | 1.618 | 1.784 |
| TCF7L2 | 688358II [symbol] | 0.0035 | −0.6005 | 0.4045 | −1.368 | 0.0785 | −0.1605 | −0.3065 | 0.6945 | 0.14 | 1.331 | −0.0135 | 1.162 | −0.7525 | −0.0005 | 0.1805 |
| TDRD7 | 695761II [symbol] | −0.033 | −0.567 | −1.696 | 0.493 | 0.523 | −0.18 | 1.189 | 0.593 | 0.19 | −1.056 | 0.684 | −0.816 | −0.38 | 0.423 | 1.442 |
| TESK2 | 685924II [symbol] | 0.2208 | −0.8262 | 3.509 | −0.1582 | −0.5692 | 0.5718 | 4.079 | 1.084 | 0.41 | −1.014 | −0.0263 | 3.528 | −0.5972 | −1.041 | −0.8362 |
| TFAP2B | 686514II [symbol] | 1.856 | −3.456 | −4.337 | 0.69 | −3.402 | 1.046 | −4.149 | −2.709 | 1.91 | −3.137 | 0.114 | −0.286 | −3.9 | 0.654 | −3.814 |
| TFCP2L1 | 685915II [symbol] | −0.401 | −0.814 | 2.605 | 0.642 | −1.435 | 0.352 | 0.492 | 3.028 | 0.29 | 0.116 | −0.89 | 0.897 | −1.098 | −1.812 | 2.455 |
| TFCP2L2 | 697960II [symbol] | 2.374 | 2.398 | 1.224 | 0.767 | 1.032 | 0.331 | 1.122 | 2.718 | 0.47 | 3.019 | 1.419 | 1.917 | 0.806 | 2.422 | 2.922 |
| TFF1 | 688687II [symbol] | 1.018 | −1.339 | 2.113 | −0.147 | −0.89 | −0.003 | 0.612 | 1.094 | 2.82 | 0.296 | −0.8 | 1.394 | 0.014 | 0.422 | 0.9 |
| TFF3 | 695605II [symbol] | 2.494 | 1.899 | 0.353 | 2.299 | 1.244 | 2.805 | 0.431 | 0.937 | 0.87 | 0.136 | 0.938 | −0.133 | 1.331 | 0.462 | 0.795 |
| TFRC | 693432II [symbol] | −1.396 | −1.776 | −1.718 | −3.329 | −2.322 | −1.63 | −0.657 | 2.03 | −0.27 | −0.389 | −1.758 | −1.883 | −1.868 | −0.069 | −0.431 |
| TGFBR3 | 690641II [symbol] | −0.611 | −0.921 | 0 | 0.729 | 0.1366 | 0.278 | −0.161 | 0.501 | 0.36 | 0.34 | −0.277 | 0.423 | −0.177 | −0.731 | −1.028 |
| THY28 | 687185II [symbol] | 2.22 | 1.072 | 1.139 | −0.037 | −0.094 | 0.498 | 1.449 | 1.502 | 0.45 | 0.297 | 1.025 | 1.445 | 0.486 | 1.831 | 0.731 |
| TIF1 | 682191II [symbol] | 0.0155 | 0.7135 | 0.2025 | −0.1365 | −0.2885 | −0.2975 | 0.1305 | 0.6745 | −0.28 | 0.6965 | −0.1135 | 0.3805 | −0.2075 | −0.0795 | −0.5025 |
| TIGA1 | 689331II [symbol] | −0.1625 | −1.146 | 0.9535 | 0.6755 | −0.2345 | −0.1825 | −0.5305 | −1.21 | 0.56 | 0.0635 | −0.1795 | −1.152 | −0.5255 | 0.7145 | −0.4805 |
| TIM14 | 681121II [symbol] | −0.452 | −0.336 | −0.044 | 1.208 | 0.661 | 0.12 | −0.406 | −0.064 | 0.28 | −1.393 | 0.18 | −0.775 | −0.584 | 0.377 | 1.077 |
| TIMELES [symbol] | 689108II [symbol] | −0.1335 | 0.7755 | −0.2025 | 0.0045 | −0.3935 | 0.4445 | 0.4155 | 0.3065 | −0.56 | 0.5305 | 0.1615 | 0.7295 | 0.7675 | −0.4085 | 0.8135 |
| TIMM9 | 684722II [symbol] | −0.178 | −0.555 | 0.93 | 0.186 | −0.05 | −0.312 | −0.111 | 0.18 | 0.21 | 1.361 | −0.174 | −0.681 | 0.133 | −0.154 | 0.053 |
| TINP1 | 688051II [symbol] | −0.3588 | 0.0103 | −0.0578 | 0.6892 | −0.0128 | −0.1258 | 0.2972 | −0.4418 | 0.15 | 0.1562 | 0.6842 | −1.171 | 0.4932 | 0.3522 | −0.1918 |
| TIP120B | 686908II [symbol] | −0.7955 | −1.166 | −0.3515 | −0.6315 | −1.692 | −0.0575 | 0.7915 | −0.1525 | 0.29 | −1.157 | 0.0575 | −0.5765 | 1.119 | −1.034 | −0.2255 |
| TK1 | 686069II [symbol] | 0.817 | 0.732 | 0.273 | 0.563 | 1.051 | 0.375 | 0.053 | 1.184 | −1.39 | 0.788 | −0.343 | 1.397 | −0.257 | −1.005 | 0.276 |
| TLE2 | 690798II [symbol] | −0.7665 | 0.9705 | −0.9055 | 0.1125 | −1.208 | 0.4925 | 0.2575 | −0.2525 | 0.2 | 0.6855 | 0.2625 | −0.2135 | −0.0155 | 0.3765 | −1.389 |
| TLR5 | 682772II [symbol] | −0.5235 | −0.3105 | 1.284 | 0.1115 | −1.029 | −0.0695 | 1.552 | 1.581 | 0.1 | −1.396 | −0.0535 | 0.7175 | −0.2965 | 0.4095 | 0.8985 |
| TM4SF12 | 689572II [symbol] | 0.1235 | 0.3255 | 0.9765 | 1.279 | −0.9505 | −1.224 | 0.0055 | 0.7385 | 0.31 | −0.5715 | −0.8685 | −0.6235 | 0.3535 | −0.9145 | 0.2395 |
| TM4SF13 | 696352II [symbol] | 0.836 | 0.515 | −1.097 | 1.717 | −0.273 | −0.199 | −0.641 | −0.159 | 0.32 | −0.315 | 0.994 | −0.768 | 0.939 | 1.724 | 0.169 |
| TMEM14A | 683793II [symbol] | −0.351 | −0.499 | −0.008 | 0.337 | −0.859 | −0.802 | −0.178 | 0.996 | 0.06 | 0.243 | 0.565 | 0.822 | 0.812 | 0.431 | 1.179 |
| TMEM25 | 697985II [symbol] | 1.289 | 2.041 | 1.604 | 4.452 | 4.117 | 2.103 | 1.263 | 1.232 | 0.42 | 0.38 | 3.018 | 2.409 | 0.398 | 2.341 | 0.898 |
| TMEM4 | 695931II [symbol] | −0.278 | −0.068 | −0.807 | −0.102 | −0.174 | −0.601 | −0.027 | −0.62 | −0.01 | 0.212 | −0.35 | 0.713 | −0.446 | −0.071 | 0.002 |
| TMPRSS5 | 689589II [symbol] | 0.062 | −1.281 | −0.508 | −0.101 | −0.866 | 0.365 | 0.216 | 0.888 | 0.28 | −1.022 | −0.442 | 1.084 | −0.259 | −0.262 | 2.376 |
| TMSB10 | 695386II [symbol] | 0.151 | −2.121 | 0.99 | 2.568 | −0.359 | 0.331 | −1.877 | 2.537 | −0.09 | −1.338 | −2.187 | −0.436 | 1.034 | −0.748 | −1.334 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| TMSNB | 681721II [symbol] | 0.775 | −1.886 | 2.735 | −0.255 | −1.544 | −1.274 | 1.195 | 2.565 | −0.71 | −0.715 | −0.88 | 0.84 | −0.718 | −1.398 | −0.055 |
| TNFRSF1 [symbol] | 681592II [symbol] | 2.846 | −1.763 | −1.022 | −0.587 | 0.682 | −0.441 | 1.566 | −0.955 | −0.21 | −1.242 | −0.239 | 0.556 | 0.049 | 0.124 | 3.136 |
| TNFRSF2 [symbol] | 689952II [symbol] | −0.1265 | −1.047 | 0.4885 | −0.3675 | −0.5095 | −0.3985 | −0.2835 | 0.1395 | 0.31 | 0.5815 | −1.228 | 1.035 | 0.4155 | 0.4305 | 0.3275 |
| TNFRSF2 [symbol] | 692465II [symbol] | 0.396 | −0.186 | 0.326 | −0.182 | 0.48 | −1.068 | −0.69 | 1.204 | 0.37 | −0.493 | −0.841 | 0.076 | −0.162 | 0.295 | −0.7 |
| TNFSF4 | 694020II [symbol] | 0.634 | 0.945 | −0.143 | −0.038 | 0.984 | −0.575 | −0.373 | −0.652 | −0.11 | −0.3 | 1.722 | −0.339 | 1.051 | 0.604 | 0.957 |
| TNFSF7 | 684958II [symbol] | 3.641 | −0.8485 | −0.8645 | −0.3235 | −0.8875 | −0.0615 | −0.6245 | 0.0615 | 0.79 | −0.7965 | 1.088 | 0.5195 | −0.3935 | 3.543 | −0.7175 |
| TNIK | 695447II [symbol] | 1.064 | −0.803 | 0.121 | 0.659 | 0.731 | −0.005 | −1.295 | 0.071 | −0.06 | −0.794 | 0.481 | 0.22 | 0.871 | 0.383 | 0.174 |
| TNNC2 | 687349II [symbol] | −0.0088 | 1.58 | −0.5958 | −0.4178 | −0.2888 | −0.1258 | 0.1732 | −0.6728 | −0.17 | −0.1358 | −0.8108 | 0.6772 | −0.1408 | −0.3086 | 1.962 |
| TNNI2 | 687679II [symbol] | 1.905 | 2.313 | 1.405 | 1.983 | 0.06 | 2.088 | −0.239 | 0.43 | 1.32 | 0.395 | 2.628 | 0.119 | 1.749 | 2.12 | 1.074 |
| TNNI3 | 695629II [symbol] | −1.783 | −0.003 | −2.565 | −0.84 | 0.513 | 0.117 | −1.975 | −0.178 | 0.02 | 2.118 | −1.95 | −1.349 | −0.372 | −0.96 | −2.018 |
| TOB1 | 683402II [symbol] | 0.1375 | 1.065 | −0.8275 | 0.4575 | −0.5385 | −0.4465 | −1.125 | −0.0355 | −0.24 | −1.417 | 0.1635 | −0.2045 | 0.2765 | 0.0575 | −0.4995 |
| TOM1L1 | 685320II [symbol] | −1.202 | 0.6715 | −0.5145 | −0.0355 | −0.0165 | 0.3445 | −1.529 | 0.5425 | 0.49 | −3.394 | 0.4865 | −1.219 | 0.5145 | 2.651 | 0.4865 |
| TOMM7 | 683060II [symbol] | −0.484 | −0.479 | −0.621 | 0.388 | −0.066 | −0.292 | −0.677 | −0.152 | 0.24 | 0.456 | −0.382 | −0.405 | −0.347 | −0.322 | 0.016 |
| TOP1MT | 693021II [symbol] | −0.49 | 0.027 | 0.921 | −0.351 | −0.567 | 1.118 | 1.395 | 1.501 | −0.13 | 0.744 | −1.549 | 0.3 | 0.126 | −1.116 | −0.46 |
| TOP2A | 690278II [symbol] | 1.801 | 0.7815 | 0.4395 | −0.3965 | 0.1995 | 0.0525 | 0.9335 | 2.232 | −0.75 | 1.564 | 0.4855 | 0.8985 | 0.7955 | 3.072 | 1.333 |
| TOP2B | 687326II [symbol] | −0.587 | −0.064 | 0.67 | 0.036 | −0.305 | 0.856 | −0.236 | −0.141 | 0.46 | −0.433 | −0.077 | −0.826 | −0.902 | −0.024 | 0.321 |
| TOPBP1 | 697492II [symbol] | −0.738 | 0.02 | −0.933 | −0.881 | −0.084 | −0.993 | −1.214 | −0.054 | −0.28 | 0.424 | −0.647 | −0.315 | −1.493 | −1.194 | 0.048 |
| TOPK | 684492II [symbol] | −0.257 | 0.083 | 1.523 | −0.063 | 0.089 | 0.569 | 2.159 | 2.617 | −1.46 | 1.332 | 0.373 | 0.848 | 0.476 | −0.726 | 1.804 |
| TP53AP1 | 688947II [symbol] | −0.602 | −0.619 | −0.402 | 0.631 | 0.519 | 0.632 | −0.528 | −0.383 | 0.49 | −1.531 | 0.085 | −2.023 | 0.665 | −0.34 | −0.645 |
| TP53BP2 | 681831II [symbol] | −0.2965 | 0.1045 | 2.468 | −0.0995 | −0.7185 | −0.6895 | 1.473 | 1.864 | 0.21 | −0.9855 | −0.3925 | 2.187 | −0.4595 | 0.2645 | 1.213 |
| TP53INP1 | 686814II [symbol] | 0.887 | 0.287 | −2.21 | 0.002 | 0.147 | 2.016 | −0.39 | −0.913 | 0.33 | −1.946 | 0.269 | −1.26 | −0.46 | 1.092 | −0.366 |
| TPD52L1 | 684176II [symbol] | −0.1945 | −0.0995 | 0.0425 | −0.1535 | −0.3185 | 0.2305 | 0.3235 | 0.4015 | 0.25 | 1.612 | 0.3315 | 0.1795 | −0.1785 | −0.0865 | −0.2035 |
| TPRT | 695363II [symbol] | −0.892 | −1.389 | 0.574 | −1.221 | −1.505 | −1.791 | −0.347 | 0.027 | −0.1 | 0.058 | −1.969 | −1.032 | −0.495 | −0.804 | 0.574 |
| TRA@ | 696810II [symbol] | 0.6835 | 0.0375 | −1.067 | −0.4275 | 1.696 | 0.7605 | −0.1295 | −0.2935 | −0.96 | −1.318 | −0.0405 | −0.2465 | −0.7685 | 0.5645 | −1.003 |
| TRAF4 | 695150II [symbol] | 0.829 | 0.859 | 1.056 | −0.011 | 0.848 | 0.423 | 0.343 | 1.025 | −0.1 | −0.329 | 0.759 | 1.28 | 0.335 | 0.459 | 0.341 |
| TRAPPC4 | 682554II [symbol] | 0.283 | 0.174 | −0.154 | 0.228 | 0.553 | 0.576 | −0.534 | −0.265 | 0.09 | −1.039 | −0.224 | −0.389 | 0.402 | 0.048 | −0.706 |
| TRH | 688633II [symbol] | −0.099 | 0.571 | −0.512 | −0.145 | 0.044 | 4.302 | −0.475 | 0.138 | −0.16 | 0.376 | 0.566 | −0.044 | −0.178 | −0.149 | −0.317 |
| TRIM17 | 684736II [symbol] | 0.3487 | 0.4867 | 1.434 | −0.5793 | −0.3983 | 0.2967 | 0.6287 | −0.5713 | 0.56 | −0.5653 | 0.8257 | 0.3857 | −0.4753 | 2.521 | 0.0388 |
| TRIM2 | 682053II [symbol] | 0.0118 | −2.233 | 1.493 | −0.0473 | −0.1012 | −0.2042 | −0.0403 | 1.233 | 0.44 | −0.9362 | −0.6722 | 3.094 | −0.8102 | −1.136 | 0.1548 |
| TRIM6 | 682620II [symbol] | 0.216 | −0.931 | −1.092 | 0.316 | −0.666 | −0.148 | −0.356 | −1.027 | 0.34 | 0.172 | 0.946 | −0.596 | −0.996 | 0.197 | 0.431 |
| TRIP3 | 696359II [symbol] | −0.906 | 0.111 | −0.931 | −0.564 | −0.264 | −0.698 | −1.045 | −0.708 | 0.15 | −0.151 | −0.246 | −0.835 | −0.38 | −0.951 | −0.438 |
| TRPS1 | 683107II [symbol] | −0.9335 | 0.4625 | 0.2285 | 0.4615 | 1.035 | 1.257 | 0.5615 | 0.7755 | 0.21 | −4.797 | −0.6465 | 0.7345 | −0.5205 | −0.4025 | −1.06 |
| TSG101 | 685755II [symbol] | −0.1475 | 0.7725 | −0.0115 | 0.3565 | 0.6845 | 0.8895 | −0.3645 | −1.446 | 0 | −0.3975 | 0.7285 | −0.4695 | −0.1315 | −0.0145 | 0.0115 |
| TTC10 | 687943II [symbol] | −0.2825 | 0.4795 | 0.0275 | 0.8875 | 1.091 | −0.4945 | −1.003 | −0.4135 | 0.71 | −0.5045 | −0.0475 | −0.8145 | −0.2985 | 0.0415 | −0.7505 |
| TTC12 | 682598II [symbol] | −0.001 | −0.269 | 1.102 | −0.018 | 0.02 | −0.219 | −0.485 | −0.763 | 0.7 | −0.097 | 1.094 | 0.204 | 0.001 | −0.429 | 0.169 |
| TTK | 691976II [symbol] | 0.678 | 0.89 | 0.733 | −0.044 | 0.356 | −0.567 | 0.705 | 1.244 | −1 | 0.68 | −0.351 | 0.225 | 0.631 | −0.061 | 0.622 |
| TTLL4 | 685109II [symbol] | 0.1645 | −0.6855 | 2.598 | −0.5045 | 0.0325 | 0.1325 | −0.3185 | 0.4825 | 0 | 0.4725 | −0.2985 | 0.3575 | 0.0185 | 0.0955 | 0.5955 |
| TTYH1 | 688941II [symbol] | 1.121 | −0.0935 | 0.7215 | −0.0085 | −1.709 | −0.0345 | 1.543 | 1.104 | 0.47 | 0.4645 | −0.8275 | 3.214 | −0.0235 | −0.0735 | 4.113 |
| TUBB3 | 685420II [symbol] | 0.006 | −0.272 | −0.628 | 0.332 | −1.184 | 0.205 | 1.72 | 1.757 | 0.02 | 0.442 | 0.175 | 0.378 | 3.158 | −0.768 | −0.593 |
| TULP3 | 684931II [symbol] | −0.36 | 0.217 | 2.027 | −0.055 | −0.04 | 0.451 | −0.209 | 1.066 | 0.04 | −0.318 | −0.549 | 0.075 | −0.156 | 0.225 | 0.406 |
| TXN | 687357II [symbol] | 0.169 | 1.25 | −0.143 | −1.205 | −0.006 | 1.373 | 0.2 | 1.312 | −0.54 | −3.489 | 0.006 | −1.581 | −0.087 | 1.525 | 0.475 |
| TYMS | 763590II [symbol] | −3.357 | −2.92 | −2.807 | −3.951 | −3.58 | −2.74 | −1.34 | −1.93 | −1.04 | −1.838 | −2.6 | −2.84 | −3.13 | −3.98 | −0.838 |
| U5-116KD | 696228II [symbol] | −0.823 | −0.015 | −0.846 | −0.929 | 0.112 | −0.036 | −0.435 | 0.003 | 0.01 | 0.195 | −0.408 | 0.062 | −0.538 | −1.186 | −0.542 |
| UBE2E3 | 682401II [symbol] | −0.8132 | −1.39 | 1.221 | −2.032 | −2.697 | 0.3708 | 0.1198 | 0.1038 | 0.81 | −0.3832 | 0.4348 | 0.0718 | 0.1188 | −1.374 | 1.29 |
| UBL5 | 688745II [symbol] | 0.462 | −0.834 | 0.531 | 0.95 | 0.025 | −0.304 | −0.093 | −0.59 | −0.01 | −0.497 | −0.214 | −0.564 | 0.578 | 0.201 | 0.187 |
| UBN1 | 687671II [symbol] | 0.3765 | 0.5135 | 0.2735 | 0.2785 | 0.0695 | 0.4975 | 0.0035 | −0.0115 | −0.14 | −0.1765 | −0.6615 | 0.4335 | −0.3785 | −0.1605 | 0.0425 |
| UCHL1 | 688791II [symbol] | −0.6 | −0.589 | 0.341 | −0.644 | −0.579 | −0.463 | 4.428 | 1.686 | −0.53 | 0.798 | 0.764 | 3.475 | −0.396 | −0.36 | 3.864 |
| UCHL3 | 695206II [symbol] | −0.061 | −1.042 | −0.134 | −0.509 | −0.134 | −1.228 | −0.513 | 0.224 | 0 | 0.585 | −0.722 | −0.7 | 0.135 | −0.486 | −0.292 |
| UGCG | 682939II [symbol] | −0.5385 | 1.11 | −1.925 | 0.8545 | 0.1905 | 2.236 | −1.134 | −1.917 | 0 | −0.3245 | 1.384 | −2.554 | 0.1985 | −0.4715 | −0.0725 |
| UGCGL1 | 686132II [symbol] | −0.457 | 0.073 | −0.486 | −0.449 | 0.666 | −0.657 | 0.053 | −0.703 | −0.76 | 0.067 | −0.687 | −1.028 | −0.327 | −0.134 | −0.546 |
| UGT2B7 | 689581II [symbol] | 1.083 | −1.669 | 0.195 | 0.414 | −0.739 | 0.452 | −1.031 | 0.991 | 4.04 | −0.891 | −0.558 | 0.024 | −0.976 | 1.051 | −0.121 |
| UNC13D | 693680II [symbol] | 1.516 | 0.811 | 1.851 | −0.533 | 0.147 | 0.683 | 1.498 | 0.271 | −0.04 | 0.484 | 0.41 | 0.675 | 1.6 | 0.033 | 0.99 |
| VAMP8 | 685078II [symbol] | 0.146 | −0.315 | 0.925 | −0.412 | −0.128 | −0.074 | 0.998 | −0.065 | 0.11 | 0.52 | −0.526 | 0.288 | 0.363 | −0.16 | 0.561 |
| VAPA | 686453II [symbol] | −0.587 | −0.532 | −0.724 | 0.105 | −0.127 | −0.277 | −0.456 | −0.04 | 0.23 | 0.279 | 0.202 | −0.107 | −0.338 | −0.104 | 0.299 |
| VAV1 | 693880II [symbol] | 0.339 | −0.451 | −1.459 | −0.517 | −0.646 | 0.023 | −0.189 | −0.92 | −0.44 | −0.776 | −0.107 | −0.183 | −0.127 | −0.382 | 0.421 |
| VAV3 | 695745II [symbol] | 1.278 | 3.255 | −0.787 | 3.543 | −1.372 | 2.015 | −0.55 | 0.875 | 1.15 | 0.517 | 3.601 | −0.159 | 1.917 | 2.901 | 0.168 |
| VCX3 | 688317II [symbol] | −0.128 | 3.141 | −0.205 | −0.097 | 2.003 | 0.286 | −0.011 | −0.578 | 0.54 | −0.214 | 1.898 | −0.518 | 0.788 | 0.45 | −0.319 |
| VDAC2 | 681387II [symbol] | 0.2585 | −0.1395 | 1.694 | 0.1125 | −0.0305 | −0.1305 | 0.1195 | 0.5895 | −0.22 | 0.6655 | 0.3455 | −0.2165 | 0.2075 | 0.4245 | 0.9915 |
| VEST1 | 685356II [symbol] | −0.0225 | 0.5425 | −0.2395 | 3.052 | 0.2325 | 3.016 | −0.0435 | 0.1295 | 0.04 | −0.2295 | 0.0525 | 0.0305 | −0.1395 | −0.1025 | −0.0455 |
| VGLL1 | 763309II [symbol] | 0.8785 | −0.2635 | −0.6895 | 0.1705 | 0.2865 | −0.0565 | 0.4605 | 0.7425 | −0.18 | 0.5705 | −1.196 | −0.1855 | −0.0745 | −1.098 | −0.1195 |
| VMD2L2 | 696188II [symbol] | 0.602 | −0.848 | 1.105 | 0.395 | 0.448 | 0.246 | 0.317 | −0.011 | 0.19 | 0.077 | 0.435 | 0.482 | 0.077 | 0.536 | 0.16 |
| VSX1 | 692722II [symbol] | 0.312 | 0.821 | 0.298 | 0.793 | 0.383 | 0.488 | 0.845 | 2.477 | −0.2 | 0.017 | 0.315 | 2.832 | −0.066 | 0.092 | 0.483 |
| WARP | 683039II [symbol] | −0.9485 | −0.1775 | −0.5135 | 0.5735 | 0.3845 | −0.2405 | 0.0915 | 0.9195 | 0.33 | 0.6095 | 0.2825 | 0.4115 | 0.5045 | 0.0555 | −1.892 |
| WDR26 | 691481II [symbol] | 0.0735 | 0.6425 | 1.001 | −0.0265 | −0.1005 | −0.2195 | −0.0095 | 0.4095 | 0.03 | −0.5015 | −0.0455 | 0.5245 | −0.0175 | 1.208 | 0.2015 |
| WDR6 | 693627II [symbol] | −0.474 | −0.061 | −0.177 | 0.707 | 0.347 | 0.644 | 0.09 | −0.49 | 0.4 | 0.013 | −0.653 | −0.227 | −0.044 | −0.902 | −0.588 |
| WHSC1L1 | 695912II [symbol] | −0.231 | 1.106 | 0.173 | 0.585 | 1.14 | 1.05 | −0.136 | 0.132 | 0.15 | 0.259 | 0.158 | 0.513 | 0.331 | 0.448 | 0.436 |
| WNT3 | 686424II [symbol] | −1.223 | −0.224 | −0.769 | 1.111 | 0.265 | 0.915 | −0.661 | 0.02 | 0.03 | 0.706 | −0.878 | 0.031 | −0.414 | −0.572 | 0.051 |
| WNT6 | 763326II [symbol] | 0.104 | −0.33 | 1.974 | −0.347 | −0.02 | −0.54 | 0.485 | 2.228 | −0.1 | 2.798 | −0.35 | 2.05 | −0.44 | −0.51 | 0.711 |
| WSB2 | 693380II [symbol] | 0.3205 | −1.655 | 0.3455 | −0.0145 | −1.316 | 2.383 | 0.1395 | −0.1315 | 0 | 0.4115 | −0.5675 | −0.0055 | 0.0025 | 0.4765 | −0.4075 |
| WWP1 | 686545II [symbol] | −0.4173 | 2.735 | −0.9713 | 0.8387 | 0.0288 | 2.384 | −0.0973 | −0.4703 | 0.34 | −1.457 | 0.4087 | −0.0943 | −0.1403 | −0.3403 | −0.7593 |
| WWP2 | 691487II [symbol] | −0.345 | 0.899 | −1.13 | −0.649 | 0.581 | −0.508 | 0.909 | 0.169 | −0.2 | −0.001 | −0.323 | 0.032 | 0.074 | −0.0834 | −0.492 |
| XBP1 | 689595II [symbol] | −0.075 | 1.484 | −4.077 | 1.272 | 0.484 | 0.947 | −2.754 | −2.815 | 0.9 | −3.277 | 1.149 | −3.168 | 0.597 | 0.223 | −1.102 |
| XYLT2 | 692339II [symbol] | −0.361 | 0.881 | −0.679 | −0.006 | 0.289 | 0.032 | −0.429 | −0.169 | 0.14 | 0.062 | 0.463 | −0.337 | −0.527 | −1.095 | −0.612 |
| YBX2 | 690818II [symbol] | 0.0105 | 0.8045 | −0.6025 | −0.4505 | 0.0295 | 1.362 | 1.943 | 0.6665 | −0.55 | −0.2705 | −0.7325 | 0.3295 | −1.371 | 0.9675 | −1.047 |
| YIF1 | 687006II [symbol] | 0.138 | −0.08 | 0.269 | −0.014 | 0.43 | −0.394 | −0.049 | −0.284 | −0.47 | 0.173 | 0.082 | −0.16 | −0.512 | 0.484 | 0.441 |
| ZA20D3 | 694225II [symbol] | −0.965 | −0.722 | −1.072 | −0.854 | −1.236 | −0.039 | −1.889 | 0.456 | 0.25 | 0.066 | −0.022 | −1.424 | 0.231 | 0.143 | −0.603 |
| ZAP1Z8 | 697114II [symbol] | −0.4613 | −0.0383 | 0.7427 | −0.2443 | −0.0483 | −0.1903 | 0.6227 | 0.9217 | −0.02 | 1.055 | 0.4387 | 0.2907 | 0.0458 | −0.6313 | 1.69 |
| ZBED3 | 694304II [symbol] | 0.135 | −0.431 | 0.797 | −0.144 | 0.284 | 0.982 | −0.23 | 0.126 | −0.11 | −1.901 | 0.386 | 0.019 | −0.465 | −0.524 | −0.625 |
| ZBTB4 | 692679II [symbol] | 0.684 | 1.728 | 0.439 | 1.606 | 1.964 | 1.654 | 0.528 | 0.61 | 0.69 | −0.272 | 1.948 | 0.52 | 1.055 | 0.609 | 0.298 |
| ZCWCC1 | 694671II [symbol] | 0.51 | 0.91 | 0.802 | 0.123 | 0.732 | 0.97 | 1.385 | 0.984 | −0.04 | −0.049 | 0.356 | 1.78 | 0.403 | 0.423 | 1.005 |
| ZCWCC2 | 688993II [symbol] | 1.088 | −0.4185 | 0.6305 | −0.4065 | −0.2515 | −0.5805 | 0.7805 | 1.064 | 0.06 | 0.8955 | −0.3515 | −1.201 | 0.3265 | 1.369 | 0.2285 |
| ZDHHC13 | 692867II [symbol] | −0.042 | −0.2218 | 1.211 | −0.085 | 0.064 | −0.77 | 0.465 | 0.41 | 0.03 | −0.776 | −0.073 | −0.429 | −0.857 | 0.193 | 0.987 |
| ZDHHC14 | 690817II [symbol] | −1.001 | 0.929 | 3.309 | −0.924 | 1.258 | −0.546 | 0.129 | 5.485 | 0.17 | −0.401 | 1.748 | 0.278 | 0.053 | −0.294 | 5.71 |
| ZFHX4 | 696157II [symbol] | 0.391 | 1.931 | −0.92 | 0.314 | 0.196 | 0.012 | −0.11 | −0.396 | 0.25 | −0.005 | 1.715 | 1.494 | 0.114 | 0.034 | 0.956 |
| ZFYVE21 | 696639II [symbol] | 0.438 | 0.712 | −0.855 | 0.465 | 0.24 | 0.726 | −0.366 | 0.713 | 0.5 | 0.289 | 0.757 | −0.086 | 0.491 | 0.014 | −0.989 |
| ZNF133 | 689721II [symbol] | −0.888 | −0.004 | 0.611 | −0.53 | −0.119 | 0.004 | −0.137 | −0.007 | 0.78 | −0.153 | 0.19 | 0.36 | −0.08 | 0.117 | 0.11 |
| ABAT | 682378II [symbol] | −0.71 | −0.17 | −1.718 | −0.198 | 2.101 | 1.742 | −2.219 | −2.788 | 2.15 | −1.856 | 1.885 | −1.976 | 0.743 | −1.355 | −1.937 |
| ZNF165 | 688726II [symbol] | −0.2035 | 0.6555 | 1.011 | −0.2675 | 0.9915 | 0.8015 | 0.0545 | 0.9905 | −0.56 | 0.7355 | 0.1295 | −0.4565 | 1.436 | −0.8705 | 0.7445 |
| ZNF24 | 683142II [symbol] | −0.724 | 0.048 | −0.451 | 0.079 | 0.561 | 0.776 | −0.614 | −0.793 | 0.32 | −1.358 | 0.107 | 0.35 | −1.129 | −0.66 | −0.358 |
| ZNF318 | 682803II [symbol] | −4.404 | −0.468 | 0.703 | 0.1 | −0.265 | −0.429 | 0.156 | 0.479 | 0.57 | −0.2 | −0.375 | −0.245 | 0.2 | −0.304 | 0.441 |
| ZNF364 | 690750II [symbol] | 0.043 | 1.099 | 0.684 | 0.261 | −0.136 | −0.006 | 0.79 | 0.339 | −0.07 | −0.054 | 0.42 | 0.595 | −0.143 | −0.021 | 0.621 |
| ZNF432 | 696694II [symbol] | 0.115 | −0.581 | 0.121 | 0.079 | −0.59 | −0.349 | 0.055 | 0.017 | 0.09 | −0.103 | −0.121 | −0.07 | 4.736 | −0.039 | −0.055 |
| ZNF462 | 682655II [symbol] | 0.3475 | 0.7615 | −1.216 | −0.2895 | −0.6575 | −0.2485 | 0.2125 | 0.9976 | 0.71 | 1.115 | −0.1365 | 0.0515 | −0.4195 | −0.164 | 0.5415 |
| ZNF505 | 697563II [symbol] | −0.062 | −1.345 | 1.695 | −0.302 | −1.101 | −0.776 | 0.345 | −0.991 | −0.18 | 0.052 | −0.367 | −0.787 | −0.257 | −1.527 | 0.252 |
| ZNF552 | 681805II [symbol] | −0.777 | −0.259 | −1.221 | 1.24 | −0.409 | 0.1 | −0.618 | −1.114 | 0.56 | −0.596 | 0.79 | −1.928 | −1.997 | −0.335 | −1.303 |
| ZNF577 | 696450II [symbol] | 0.594 | 0.483 | 1.045 | 0.188 | 0.391 | 1.083 | 0.363 | 0.186 | 0.77 | 0.396 | 1.791 | −0.272 | 3.899 | 0.603 | 0.257 |
| ZNF587 | 695267II [symbol] | −0.446 | 0.393 | 0.383 | 1.546 | 0.898 | 1.35 | 0.089 | 0.408 | −0.17 | 0.392 | 1.469 | −0.226 | −0.663 | 0.457 | −0.202 |
| ZNF598 | 689656II [symbol] | 0.6632 | 0.2352 | 0.2132 | −0.5278 | 0.4472 | 0.8932 | 0.6212 | 0.5472 | −0.02 | −0.5568 | −0.3968 | 0.4472 | −0.0128 | −0.0725 | 0.8852 |
| ZNF599 | 697181II [symbol] | −0.26 | 0.012 | 2.135 | 1.661 | 0.306 | 0.977 | −0.308 | 0.171 | 0.33 | −0.172 | 0.694 | −0.418 | 0.806 | 0.103 | −0.06 |
| ZNF606 | 683801II [symbol] | 0.9348 | −0.2202 | 1.263 | −0.2632 | −0.6392 | 0.0888 | 0.0898 | −0.0083 | 0.46 | −0.5032 | 0.5348 | −0.1692 | −0.6272 | −0.1902 | 0.0268 |
| ZNF614 | 683555II [symbol] | −0.254 | 0.733 | 0.158 | −0.164 | −0.03 | 0.611 | −0.172 | −0.119 | −0.06 | 1.452 | 0.582 | −0.101 | 4.451 | −0.016 | 0.294 |
| ZNF639 | 690004II [symbol] | −1.119 | −0.862 | 0.172 | −0.547 | −0.225 | −0.847 | −1.019 | 0.412 | 0.1 | −0.658 | −0.356 | −1.029 | −0.846 | −0.49 | −0.513 |
| ZNF7 | 684715II [symbol] | 1.521 | 2.282 | 1.746 | 2.8 | 2.387 | 2.442 | 0.777 | 1.214 | 0.24 | −0.767 | 1.321 | 2.685 | 1.835 | 1.378 | 1.659 |

| Symbol | NAME | PB362 | PB370 | PB376 | PB413 | PB441 | PB455 | UB29 | UB37 | UB38 | UB39 | UB43 | UB45 | UB55 | UB57 | UB58 | UB60 |
|---|---|---|---|---|---|---|---|---|---|---|---|---|---|---|---|---|---|
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| BIRC2 | 685336II [symbol] | −0.0115 | −0.4705 | −0.3475 | 0.2515 | −0.0345 | 0.1045 | 0.5875 | 0.147 | 0.1 | −0.2855 | 0.4625 | −0.0105 | −0.303 | 0.0275 | 0.0265 | 0.1595 |
| BIRC5 | 687015II [symbol] | −1.034 | −0.1875 | 1.586 | −0.0985 | −0.7355 | 1.556 | 1.746 | 2.579 | −0.558 | 0.3125 | 0.4145 | −0.3085 | −0.1445 | −1.023 | −0.0475 | −0.2085 |
| Bit1 | 685275II [symbol] | −0.9605 | −0.3305 | 0.2935 | 0.0635 | −0.5085 | 1.881 | 1.118 | −0.508 | 0.1275 | 0.4375 | −0.6625 | 0.2825 | −0.3275 | −0.0005 | −0.5205 | 0.8225 |
| BIVM | 691115II [symbol] | −0.0215 | −0.3935 | −0.0655 | 0.1415 | −1.253 | −0.5095 | 0.4205 | −1.832 | −0.4105 | 0.2265 | 0.4155 | 0.3435 | 0.766 | −0.2055 | 1.024 | −0.0805 |
| BLVRA | 695027II [symbol] | −0.614 | −0.5 | 0.149 | 0.063 | 0.203 | −0.053 | −1.145 | 0.1625 | 0.875 | 0.586 | 1.057 | −0.203 | 0.0785 | −1.136 | 0.599 | 0.308 |
| BM039 | 692354II [symbol] | −3.488 | −2.849 | −1.676 | −3.056 | −3.501 | −2.385 | −1.912 | −2.931 | −3.179 | −3.045 | −2.513 | −3.334 | −3.266 | −2.763 | −2.706 | −3.111 |
| BMP7 | 697100II [symbol] | −0.396 | 0.633 | 1.718 | 0.01 | −0.99 | 0.524 | 1.121 | 1.8375 | −0.4765 | 0.102 | 1.504 | −0.393 | −0.274 | −0.067 | 0.579 | 1.241 |
| BOC | 694925II [symbol] | −0.443 | −0.192 | 0.304 | −0.039 | 0.247 | −0.237 | 0.555 | 0.1975 | −0.4875 | −0.571 | −0.357 | −0.017 | −0.451 | −0.382 | −0.025 | −0.036 |
| BRCA1 | 691229II [symbol] | −0.68 | −0.633 | 1.694 | 0.07 | −0.588 | −0.12 | 0 | −0.7015 | −0.6855 | 0 | −0.11 | 0.55 | 0.4855 | 0.07 | 0.168 | 0.07 |
| BRF2 | 697749II [symbol] | 0.054 | −0.278 | −0.77 | −0.72 | 0.178 | 0.13 | 0.771 | 0.1 | 0.1845 | 0.342 | 0.932 | 0.65 | 0.2915 | 0.115 | −0.05 | −0.135 |
| BRI3 | 694633II [symbol] | 0.164 | 0.106 | 0.101 | 0.447 | 0.035 | 0.269 | −0.024 | 0.2895 | −0.146 | −0.112 | 0.267 | −0.239 | 0.3445 | 0.189 | 0.228 | −0.045 |
| BRI3BP | 685224II [symbol] | −0.0735 | −0.2695 | −0.2595 | 0.2025 | 0.1825 | −0.3285 | 0.4905 | −0.502 | −1.073 | 0.2235 | 0.7045 | −0.3415 | −0.6565 | 0.3025 | 0.1055 | −0.3045 |
| BSPRY | 689357II [symbol] | −1.528 | 1.364 | 1.443 | −0.0855 | 0.0615 | −0.9235 | −0.4895 | −0.5055 | −0.4415 | 0.2555 | 1.016 | −0.1735 | −0.3465 | 0.3265 | −0.5055 | 0.0245 |
| BTF3 | 686148II [symbol] | 0.115 | 0.093 | −0.842 | 0.093 | −0.143 | −0.267 | −0.772 | −0.9595 | −0.3765 | 1.043 | 0.707 | 0.531 | 0.574 | 0.534 | −0.004 | 0.419 |
| BTG3 | 681935II [symbol] | 0.694 | −0.423 | 0.947 | −1.106 | −0.379 | −0.293 | 1.08 | 0.229 | −1.226 | −0.978 | −0.044 | 0.88 | −1.665 | −0.15 | −0.66 | 0.721 |
| BTN2A2 | 685872II [symbol] | −0.4412 | −0.3842 | 0.6248 | 1.201 | 0.4778 | 0.2388 | 0.3908 | −0.0288 | −0.9592 | 0.0748 | −0.6812 | −1.059 | −0.4657 | −0.1052 | −0.5402 | 0.1588 |
| BUB1 | 682711II [symbol] | −1.206 | −0.0475 | 0.7055 | −0.7335 | −0.6105 | −0.0075 | 0.5046 | 0.313 | −0.7605 | −0.4445 | 0.6365 | −0.4255 | −0.186 | −1.197 | −0.3405 | 0.4435 |
| BUB3 | 683752II [symbol] | −0.6125 | 1.122 | 0.4535 | −0.0575 | 0.5595 | −0.5165 | −1.003 | −0.888 | −0.2995 | 0.1585 | 0.7055 | 1.158 | −0.2835 | 0.0815 | −0.3515 | −0.2865 |
| BXDC1 | 686690II [symbol] | −0.609 | −0.338 | 0.494 | −0.144 | 0.057 | 0.354 | 1.586 | 0.0995 | 0.9735 | −0.313 | 0.349 | −0.409 | −1.005 | −0.088 | −0.509 | −0.293 |
| BZRP | 682336II [symbol] | −0.4515 | 0.0895 | 0.9545 | −0.2055 | 0.0365 | −0.1535 | −0.1615 | 1.022 | −0.6005 | −0.8565 | −0.0365 | −0.0885 | 0.3135 | −0.1455 | −0.3405 | −0.1085 |
| BZW1 | 689148II [symbol] | 0.356 | 0.781 | −0.271 | −0.09 | −0.403 | −0.708 | −0.973 | −0.6525 | −0.858 | 0.318 | −0.235 | −0.13 | −0.387 | 0.928 | −0.42 | 0.186 |
| C10orf116 | 694348II [symbol] | 3.185 | 3.845 | 1.628 | 1.128 | 3.176 | 2.82 | 0.102 | 1.6235 | 3.165 | 3.237 | 2.978 | 3.335 | 2.749 | 3.25 | 2.503 | 1.8 |
| C10orf38 | 696429II [symbol] | 0.523 | −0.67 | −0.074 | −0.149 | −0.823 | 0.07 | 1.506 | −0.6515 | −0.5005 | −0.519 | −0.334 | −0.909 | −0.8585 | −0.265 | −0.538 | −0.014 |
| C10orf4 | 682455II [symbol] | −0.653 | −0.722 | 0.166 | −1.023 | −0.95 | −0.422 | 0.954 | −0.3155 | −0.8255 | −0.676 | 0.125 | −0.705 | −1.1885 | −1.323 | −1.126 | 0.16 |
| C18orf45 | 696197II [symbol] | −0.404 | 0.493 | 1.214 | 4.51 | 0.039 | 0.889 | 1.823 | 0.434 | 0.8235 | −0.335 | 1.427 | 0.593 | 0.808 | 0.548 | 0.881 | 1.406 |
| C10orf7 | 688194II [symbol] | −0.255 | 0.087 | 0.745 | −0.088 | −0.428 | 0.177 | 1.302 | 0.5555 | −0.089 | −0.199 | 0.564 | −0.302 | −0.4815 | −0.041 | −0.151 | 0.041 |
| C10orf82 | 693231II [symbol] | 0.857 | 3.109 | −0.127 | −0.329 | 0.148 | −0.153 | −0.186 | −0.336 | −0.1155 | 1.247 | 2.37 | 1.036 | −0.334 | −0.4 | 1.567 | 0.099 |
| C11orf24 | 694704II [symbol] | −0.339 | 0.471 | 0.142 | −0.468 | −0.525 | 0.345 | 0.307 | 0.739 | 0.097 | −0.953 | −0.016 | −0.493 | −0.2765 | 0.272 | −0.166 | −0.144 |
| C12orf14 | 692853II [symbol] | −1.03 | −0.948 | −0.399 | −1.361 | −1.354 | 0.063 | 0.113 | −0.545 | −0.0105 | −0.596 | −0.935 | −0.915 | −0.8895 | −0.734 | −0.288 | −0.701 |
| C13orf21 | 697705II [symbol] | 0.934 | 1.316 | −0.182 | 1.939 | 0.172 | 0.468 | 1.315 | 1.5345 | 1.3385 | 2.574 | 0.943 | 2.362 | 0.216 | 2.895 | 1.438 | 1.646 |
| C13orf23 | 691314II [symbol] | −1.11 | −0.4595 | −0.1585 | −0.8625 | −0.1715 | 0.4455 | 1.369 | 0.529 | 0.732 | 0.3355 | 0.9485 | −0.2605 | −0.5575 | −0.6455 | −0.3755 | 0.1235 |
| C14orf94 | 694118II [symbol] | −0.6855 | 0.7785 | 0.5315 | 0.4445 | −0.4725 | 0.5295 | 1.151 | 0.538 | −0.9385 | 0.5865 | 0.5065 | −0.1775 | −0.6505 | −0.1805 | 0.8195 | 0.2405 |
| C16orf23 | 681265II [symbol] | −1.209 | −0.4345 | 0.6735 | 0.0475 | −0.1595 | −0.7415 | −0.0895 | 0.126 | −0.561 | −0.4555 | 0.9505 | −0.6835 | −0.4735 | −0.3865 | −0.5745 | −0.1275 |
| C16orf45 | 772706II [symbol] | −0.5745 | −0.0285 | −0.1885 | 1.232 | 1.305 | 0.1535 | 0.1385 | 0.272 | −0.117 | 1.446 | 0.1005 | −0.2855 | −0.415 | −0.4175 | 0.0355 | 0.1255 |
| C17orf37 | 686313II [symbol] | −0.6392 | −0.6662 | −0.3012 | 0.3058 | 0.0188 | 1.736 | −0.4252 | 1.847 | 0.0878 | 0.6128 | −0.5552 | −0.2762 | −0.0188 | 0.4928 | −0.4002 | 2.317 |
| C18orf1 | 684420II [symbol] | −0.07 | 1.797 | −0.006 | −1.158 | 0.113 | −0.284 | 0.397 | 0.581 | −0.7545 | −0.326 | 0.088 | 0.903 | 0.5645 | −0.436 | −0.187 | 0.036 |
| C19orf10 | 687290II [symbol] | −0.0405 | 1.189 | −0.7745 | −0.9535 | −0.3715 | 3.22 | 2.801 | 1.389 | 0.667 | −0.6815 | −0.7525 | 1.807 | −0.5415 | −1.299 | 1.653 | 1.598 |
| C19orf32 | 697502II [symbol] | −0.069 | 0.912 | 0.469 | −0.096 | 2.281 | −0.031 | −0.006 | 1.4915 | 3.444 | 0.374 | 0.521 | 1.673 | 1.5535 | 1.654 | 0.678 | 0.711 |
| C19orf33 | 693018II [symbol] | −0.763 | −1.649 | −0.641 | −0.118 | −2.046 | −0.834 | −0.63 | −0.6275 | −1.0495 | 0.727 | 0.112 | −1.229 | −0.635 | 0.883 | 0.61 | 0.303 |
| C1orf24 | 689493II [symbol] | −1.371 | −0.1355 | −1.624 | −0.3585 | 1.577 | 0.5065 | −2.28 | −0.2555 | 0.2875 | 0.7655 | 0.1485 | 0.8455 | 1.1 | 0.2635 | 0.3285 | −0.2565 |
| C1orf34 | 694666II [symbol] | −0.208 | 2.409 | −0.122 | 0.157 | 1.694 | 0.757 | 0.436 | 1.5795 | 0.269 | 0.962 | 0.811 | 2.774 | 0.4445 | 0.799 | 0.88 | 1.053 |
| C20orf103 | 695553II [symbol] | 2.018 | 1.813 | −0.122 | 0.742 | 0.761 | 1.186 | 0.65 | 3.013 | 2.0255 | 1.129 | 2.311 | 1.79 | 1.7645 | 2.506 | 2.592 | 1.712 |
| C20orf149 | 691805II [symbol] | −0.4645 | 1.034 | −0.7325 | −0.8675 | 0.0205 | 0.1885 | 0.0465 | −0.323 | 0.0965 | 0.7825 | 1.142 | 0.2025 | 0.4305 | −0.0545 | 0.3405 | −0.1015 |
| C20orf23 | 697940II [symbol] | 0.559 | 2.08 | −0.419 | 1.998 | 1.46 | 0.17 | 0.336 | −0.576 | 1.2375 | 2.203 | 2.427 | 2.412 | 1.5685 | 2.078 | 1.747 | 1.566 |
| C20orf3 | 685459II [symbol] | −0.792 | 0.794 | −0.861 | 0.484 | 0.811 | 0.299 | −0.757 | −0.1095 | −0.6 | −0.234 | 0.384 | −0.254 | −0.3465 | 0.02 | −0.43 | −0.343 |
| C20orf35 | 693279II [symbol] | −0.574 | 0.676 | 0.484 | −0.311 | −0.504 | −0.251 | 0.56 | −0.643 | −1.163 | 0.255 | −0.203 | 0.001 | 0.496 | −0.449 | −0.31 | 0.296 |
| C20orf43 | 687406II [symbol] | −0.5725 | 0.4735 | 0.2555 | −0.2095 | 0.1415 | −0.5695 | 0.2195 | −0.1395 | −0.865 | 0.2345 | 0.5775 | −0.0195 | 0.0385 | −0.1065 | −0.0345 | −0.1955 |
| C20orf6 | 688651II [symbol] | 0.063 | −0.376 | −0.671 | −0.287 | −0.059 | 0.491 | 0.236 | −0.7815 | −0.471 | 0.117 | 0.51 | 0.706 | 0.3625 | −0.046 | 0.242 | 0.41 |
| C21orf45 | 681352II [symbol] | 1.235 | −1.163 | −0.795 | 1.03 | 0.024 | 0.351 | −0.878 | −0.246 | 0.8535 | 0.58 | −0.939 | −1.376 | −1.435 | −1.255 | −0.716 | −0.799 |
| C22orf18 | 681943II [symbol] | −1.253 | −0.427 | 0.871 | 0.387 | −0.577 | 0.229 | 0.362 | 0.519 | −0.0445 | −0.574 | −0.122 | −0.577 | 0.1575 | −0.619 | −1.069 | 0.022 |
| C2F | 687401II [symbol] | −0.317 | 0.161 | −0.091 | −0.077 | 0.194 | −0.202 | 0.703 | −0.174 | 0.2195 | −0.187 | 0.505 | −0.217 | 0.162 | 0.015 | −0.269 | −0.28 |
| C2orf17 | 693373II [symbol] | 0.359 | 0.905 | 0.306 | 1.204 | 0.79 | −0.014 | 0.152 | 0.604 | 0.6025 | 0.947 | 0.108 | 0.987 | 0.689 | 1.004 | 1.025 | 0.48 |
| C4A | 689714II [symbol] | −0.925 | −0.09 | −0.18 | −0.284 | 0.163 | −0.737 | −0.377 | −0.363 | 0.221 | 0.09 | 1.078 | 0.348 | −0.38 | 0.803 | 0.167 | 0.358 |
| C5orf13 | 694921II [symbol] | −1.035 | −0.801 | 0.375 | −1.193 | −1.281 | −0.634 | −0.243 | 0.1595 | −0.464 | −1.108 | 0.743 | −0.065 | −0.841 | −0.435 | −0.15 | −1.028 |
| C6orf108 | 694989II [symbol] | −0.875 | −0.797 | −0.607 | −0.029 | −1.87 | −0.439 | −0.469 | −0.271 | −0.524 | −1.07 | −0.75 | −0.74 | −0.441 | 0.146 | −0.634 | −0.545 |
| C8orf166 | 694215II [symbol] | 0.293 | −0.092 | 0.49 | −0.397 | 0.518 | 0.277 | 0.995 | 0.7865 | 0.3305 | 0.402 | 0.067 | −0.231 | −0.6335 | 0.334 | 0.132 | 0.707 |
| C8orf182 | 691969II [symbol] | 0.612 | −0.797 | 0.652 | 0.379 | 0.034 | 0.902 | 1.527 | −0.5785 | −0.061 | −0.122 | −0.644 | 0.175 | −0.761 | 0.417 | −0.169 | 0.578 |
| C6orf211 | 692990II [symbol] | −0.196 | −0.748 | 0.705 | −0.246 | −0.884 | 0.414 | −0.036 | −0.9245 | −0.5915 | 0.504 | 1.204 | 0.514 | 0.258 | −0.356 | −0.028 | 0.314 |
| C6orf49 | 692808II [symbol] | −1 | −1.208 | −0.571 | −0.911 | −0.69 | −1.034 | −0.231 | −1.1035 | −1.3135 | −0.613 | −0.134 | −0.881 | −0.78 | −0.325 | −1.034 | −0.089 |
| C6orf85 | 693055II [symbol] | 0.619 | 0.068 | 0.264 | −0.14 | 0.634 | 1.518 | −1.594 | 0.8765 | 0.827 | −0.421 | −0.332 | 0.729 | −0.0475 | 0.252 | −0.167 | 1.267 |
| C7orf24 | 685020II [symbol] | −0.4475 | −0.0785 | 1.496 | −0.7295 | −0.6855 | 0.5815 | −0.0585 | 0.6445 | −0.855 | −0.9565 | 0.1265 | 0.3285 | 0.669 | −0.9835 | −0.6775 | 0.9305 |
| C9orf10 | 682761II [symbol] | 0.148 | 0.05 | 1.404 | 0.703 | −0.025 | 1.253 | −0.59 | 0.726 | −0.2095 | 1.182 | 0.49 | 1.817 | 0.051 | 1.643 | 0.197 | 1.791 |
| C9orf121 | 692585II [symbol] | −0.605 | 1.079 | −1.696 | −2.273 | 1.569 | −2.417 | −1.828 | −0.8805 | 0.79 | −0.864 | −0.085 | 0.671 | 0.9965 | −1.508 | 0.714 | −1.275 |
| C9orf58 | 686494II [symbol] | 0.7125 | −0.9565 | 1.937 | −0.8365 | −1.023 | −0.5225 | 2.184 | 1.312 | −0.0025 | −0.3015 | 0.0775 | −2.135 | −1.06 | −1.449 | 0.0595 | 0.9045 |
| C9orf85 | 685307II [symbol] | 0.16 | −0.334 | −0.415 | −0.007 | −0.125 | 0.445 | −0.115 | 0.233 | −0.043 | −0.1 | −0.034 | −0.121 | 0.0885 | 0.003 | −0.36 | 0.027 |
| C9orf9 | 696998II [symbol] | −0.5005 | 0.3505 | 0.0735 | −1.316 | 0.7675 | 0.5055 | −0.4485 | 0.2385 | 0.2745 | −1.107 | 0.6885 | 0.3575 | 0.4165 | −1.168 | 0.0075 | −0.2395 |
| CA12 | 697341II [symbol] | 1.595 | 3.19 | −0.102 | 1.69 | 3.291 | 0.229 | −1.075 | −0.3475 | 1.7605 | 2.306 | 3.246 | 1.945 | 2.307 | 0.145 | 1.938 | 0.18 |
| CA8 | 681755II [symbol] | −0.3055 | 0.8355 | −0.6905 | −0.3195 | 0.9815 | −2.2865 | −0.1185 | −0.328 | −0.4615 | −0.5745 | −0.8925 | 1.552 | 0.668 | 1.094 | −0.1205 | −0.2545 |
| CA8 | 683844II [symbol] | −0.1655 | 0.8975 | −1.016 | −0.7535 | 0.7085 | −0.4425 | −0.6826 | −1.14 | −1.305 | −0.8535 | −1.27 | 1.352 | 0.591 | 0.7875 | −0.0445 | −0.3445 |
| CACNA1 [symbol] | 687482II [symbol] | 0 | 0.209 | −0.009 | 0.2994 | −0.177 | 0.491 | 0.147 | 0.527 | 1.348 | −0.065 | 0.253 | 0.054 | 0.0935 | 0.166 | 0.097 | 0.139 |
| CALCA | 686738II [symbol] | −0.185 | −0.048 | −0.037 | 0.13 | −0.222 | 0.118 | 0.061 | 1.433 | 0.0305 | −0.132 | 0.382 | 0.281 | 0.009 | −0.084 | 0.136 | −0.035 |
| CALML4 | 685502II [symbol] | 0.5765 | −0.4175 | 0.0755 | −0.4935 | 1.265 | 0.1585 | 1.253 | 0.937 | 2.06 | −0.3395 | −0.6995 | −0.7835 | −0.469 | −0.5885 | −0.7115 | 1.874 |
| CALML5 | 686121II [symbol] | −0.267 | −0.401 | 3.238 | −0.2826 | −0.411 | 3.985 | −0.564 | 2.481 | 0.115 | −0.311 | −0.198 | 0.45 | −0.847 | −0.646 | 0.507 | 3.125 |
| CaMKIIN [symbol] | 691045II [symbol] | −0.129 | 0.607 | −0.684 | −0.006 | 0.015 | 0.019 | −0.635 | −1.082 | −0.612 | 1.209 | 0.006 | 0.576 | 0.8025 | −0.033 | 1.185 | 0.446 |
| CAP2 | 693946II [symbol] | 0.428 | 0.908 | −1.687 | 0.091 | 1.247 | −0.201 | −0.12 | −0.275 | 1.3725 | −1.1 | −0.333 | 0.393 | 0.421 | −1.113 | −0.46 | −0.588 |
| CAP350 | 695521II [symbol] | 0.344 | −0.74 | −0.578 | −0.443 | 0.316 | 0.07 | −0.153 | −0.739 | −0.868 | 0.275 | 0.046 | 1.029 | 0.0905 | −0.463 | 0.036 | 0.468 |
| CAPN13 | 691887II [symbol] | −0.9583 | 0.8667 | −0.2213 | −0.0763 | 0.5727 | 0.6727 | −1.109 | 2.27 | −0.0308 | 0.0308 | −1.116 | 1.193 | −0.1468 | 0.8967 | −0.7243 | 0.4897 |
| CAPN6 | 697168II [symbol] | −0.013 | 0.531 | −0.88 | −1.22 | 1.259 | 0.801 | 0.135 | −0.9755 | 0.993 | 1.58 | −1.943 | −1.982 | 0.8075 | −0.141 | 0.03 | 0.939 |
| CARD14 | 681056II [symbol] | 0.042 | 0.146 | −0.32 | −1.119 | −0.765 | 2.119 | 0.461 | 0.1415 | −0.123 | 0.692 | 1.029 | −0.042 | 0.571 | −0.422 | 0.413 | −0.262 |
| CART | 687059II [symbol] | −0.2145 | −0.0595 | −0.0155 | 4.095 | −0.3065 | 0.3625 | −0.0025 | −0.2965 | 0.0275 | 0.1125 | 0.2105 | 0.0945 | 0.051 | 4.935 | −0.0926 | 0.0795 |
| CASP1 | 682686II [symbol] | 0.026 | −0.235 | 0.421 | 0.029 | −0.044 | 0.084 | 0.278 | 1.372 | −0.782 | −0.213 | 0.539 | −0.038 | −0.849 | −0.147 | −0.551 | 1.233 |
| CASP3 | 696606II [symbol] | −0.143 | 0.016 | 0.693 | −0.395 | 0.137 | 0.397 | 0.706 | −0.004 | −0.2145 | 0.489 | −0.109 | −0.242 | −0.1425 | −0.041 | 0.121 | 0.773 |
| CASP7 | 693491II [symbol] | 0.547 | 0.864 | 0.772 | 0.193 | 1.227 | 1.183 | −0.068 | 0.947 | 0.6695 | 0.443 | 1.502 | 0.684 | 0.1345 | 0.391 | 0.443 | 1.333 |
| CBLN1 | 685274II [symbol] | −0.1103 | 0.0228 | 0.1267 | 0.5137 | 0.1957 | −0.1363 | −0.1403 | −0.0228 | 0.9007 | −0.0493 | −0.2443 | −0.0893 | −0.1608 | 1.259 | 0.2317 | 0.1717 |
| CBR1 | 764397II [symbol] | 0.493 | 2.149 | 0.936 | 0.879 | 2.72 | 2.412 | 1.899 | 2.536 | 1.8815 | 1.513 | −0.185 | 2.296 | 2.2235 | 0.446 | 0.234 | 1.914 |
| CBR3 | 683488II [symbol] | −0.2025 | 0.0295 | 2.441 | −1.453 | −0.0295 | −0.6765 | 0.4775 | 1.571 | 1.574 | −1.122 | −0.3825 | 0.9245 | −0.734 | −1.269 | 0.4005 | 1.458 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| CBR4 | 697391II [symbol] | −0.932 | −0.318 | 0.866 | −0.126 | 0.752 | −0.736 | −0.215 | 0.2805 | −0.1895 | −0.009 | −0.362 | 0.072 | −0.4935 | 0.127 | −0.686 | 0.238 |
| CCL24 | 681864II [symbol] | 0.291 | 0.074 | 0.044 | 0.065 | −0.085 | 0.526 | −0.018 | 0.504 | 0.4265 | −0.228 | −0.23 | 0.048 | 0.2075 | −0.107 | 0.005 | −0.111 |
| CCL28 | 685513II [symbol] | 1.671 | −0.665 | 1.217 | −0.122 | 0.792 | −1.118 | 4.034 | 0.2605 | 1.185 | 1.657 | 0.248 | −1.349 | −1.384 | −0.969 | 0.22 | 2.778 |
| CCNB2 | 694924II [symbol] | −0.619 | −0.287 | 0.029 | 0.097 | −0.107 | −0.359 | −1.001 | −0.697 | −1.0085 | −1.379 | −1.124 | −0.377 | −0.13 | −0.734 | −0.793 | −1.021 |
| CCND1 | 689117II [symbol] | −0.012 | 0.597 | −1.878 | 0.677 | 0.487 | −1.085 | −1.311 | −0.102 | 0.526 | 2.428 | −0.149 | 0.212 | −0.563 | 1.658 | −0.0805 | −0.914 |
| CCNE1 | 685647II [symbol] | −0.0813 | −0.1662 | 1.224 | −0.2332 | −0.7842 | −0.0153 | 1.357 | 1.118 | 0.4468 | −0.2332 | −0.1932 | −0.6242 | 0.0153 | −0.6912 | −0.2312 | −0.0453 |
| CCNG2 | 686672II [symbol] | 0.1515 | 0.4855 | −1.205 | −0.1365 | 0.1365 | −0.2815 | −1.433 | −1.062 | −0.7755 | 0.9325 | 0.3845 | 1.461 | 0.549 | −0.9695 | −0.1655 | 0.3805 |
| CCNH | 688140II [symbol] | −0.1875 | −0.2475 | −0.4235 | 0.0925 | 0.5195 | 0.3495 | 0.1805 | 0.0035 | −0.0035 | 0.4545 | −1.017 | −0.2315 | −0.017 | 0.1025 | −0.5205 | −0.2355 |
| CD38 | 691273II [symbol] | −0.168 | −0.559 | 0.136 | 0.534 | 0.482 | −0.098 | −0.027 | 0.027 | −0.4135 | 0.322 | −0.488 | −0.77 | −0.7915 | −1.36 | −0.865 | 0.185 |
| CD3D | 696702II [symbol] | −1.041 | −2.571 | −1.96 | −2.195 | −2.8 | −2.101 | −2.622 | −1.917 | −3.1555 | −2.486 | −2.772 | −2.04 | −3.3125 | −3.185 | −3.182 | −1.799 |
| CD4 | 691128II [symbol] | −0.015 | −0.036 | −0.137 | 0.479 | 0.428 | 0.275 | 0.108 | 0.9325 | 0.4185 | 0.222 | 0.063 | −0.217 | −0.002 | −0.19 | 0.181 | −0.236 |
| CD58 | 695421II [symbol] | −1.304 | −1.51 | −0.586 | −1.525 | −1.316 | −0.527 | −0.142 | −1.4935 | −1.0995 | −0.408 | −0.231 | −0.558 | −1.5845 | −0.72 | −1.236 | −0.336 |
| CD59 | 682450II [symbol] | 0.2065 | 0.2785 | −0.4395 | 1.134 | 0.2495 | −1.269 | 0.4595 | −0.9665 | −0.8475 | −0.3265 | 0.9355 | −0.0005 | −1.401 | 2.685 | −0.4015 | 0.0005 |
| CDC2 | 694849II [symbol] | −4.226 | −2.8 | −1.583 | −0.575 | −2.706 | −2.259 | −2.192 | −3.225 | −3.1295 | −2.316 | −1.203 | −2.498 | −2.726 | 3.333 | −2.999 | −1.899 |
| CDC6 | 693742II [symbol] | −3.418 | −3.475 | −0.523 | −2.751 | −3.045 | 0.601 | −1.673 | −2.9005 | −2.7335 | −2.89 | −2.851 | −2.762 | −2.7075 | −2.741 | −2.745 | −2.992 |
| CDCA1 | 693999II [symbol] | −3.18 | −2.368 | −1.567 | −2.986 | −2.966 | −2.274 | −1.793 | −1.623 | −2.1815 | −0.796 | −1.982 | −2.514 | −2.2725 | −3.56 | −1.872 | −1.84 |
| CDCA5 | 696764II [symbol] | −4.174 | −1.368 | −0.583 | −2.61 | −3.384 | −1.491 | −0.992 | −1.377 | −3.4315 | −2.551 | −1.237 | −3.009 | −2.424 | −3.403 | −2.737 | −1.658 |
| CDCA7 | 686391II [symbol] | −0.8 | 0.785 | 0.54 | −0.86 | 0.002 | 1.045 | 0.298 | 0.187 | 0.5375 | −1.841 | 0.855 | 0.708 | −1.994 | −1.062 | 1.955 | −0.07 |
| CDH1 | 693949II [symbol] | −0.268 | −2.208 | 1.722 | 1.498 | 0.927 | 1.09 | 1.056 | 0.421 | −0.461 | 2.344 | 2.101 | 0.909 | −3.2125 | 0.753 | 1.94 | 1.076 |
| CDH3 | 689567II [symbol] | 0.4405 | −0.5505 | 2.17 | −0.4405 | −1.527 | 0.5745 | 1.736 | 1.165 | −1.009 | −0.1065 | 0.5095 | −0.9195 | −1.232 | −2.25 | −0.1425 | 0.1565 |
| CDK2 | 690323II [symbol] | −1.66 | −0.0125 | 1.146 | −0.6125 | 0.0015 | 0.4355 | 0.7785 | 0.6115 | 0.445 | 0.0295 | 0.1105 | −0.0435 | 0.3765 | −0.6695 | 0.2435 | −0.1235 |
| CDKN2A | 692881II [symbol] | −0.025 | 0.07 | 1.653 | 0.327 | −0.769 | 0.265 | 1.39 | 0.875 | −0.278 | −0.321 | −0.71 | −0.986 | −0.4 | −0.056 | −0.113 | −0.78 |
| CDKN2D | 682687II [symbol] | −0.163 | 0.023 | 0.104 | 0.119 | −0.59 | −0.549 | 0.401 | 0.8575 | 0.35 | 1.191 | 0.092 | −0.554 | −0.2905 | 0.015 | −0.207 | −0.628 |
| CDKN3 | 684517II [symbol] | −1.447 | 0.2375 | 0.3815 | −0.3115 | −0.6405 | 0.5175 | 0.0035 | −0.089 | −0.7515 | −0.1665 | 1.084 | 0.2915 | −0.403 | −0.4645 | −0.2875 | 0.0435 |
| CDT1 | 686706II [symbol] | −1.324 | −0.2495 | 1.401 | −0.2975 | −1.184 | 1.511 | 0.7715 | 0.7245 | −0.1295 | −0.0485 | −0.0875 | −0.6755 | −0.2585 | −0.1555 | 1.393 | −0.2735 |
| CEBPG | 688002II [symbol] | 0.3525 | −0.1475 | −0.5465 | 0.2886 | −0.0155 | 0.0155 | 0.8375 | 1.171 | −0.455 | −0.2345 | −0.3085 | −0.7985 | 0.1625 | 0.2755 | −0.3285 | 0.2025 |
| CELSR1 | 686084II [symbol] | −1.159 | 0.0005 | −1.057 | −0.8105 | 0.9365 | 0.2335 | −1.752 | −1.444 | −0.4375 | 0.4265 | 1.476 | 0.7565 | 0.918 | 0.4685 | 0.2805 | −0.6555 |
| CENPA | 697624II [symbol] | −1.483 | −1.091 | −0.569 | −2.247 | −2.225 | −1.356 | −0.558 | −1.136 | −1.5445 | −1.631 | −1.162 | −2.035 | −1.899 | −2.022 | −0.874 | −1.268 |
| CENPE | 683395II [symbol] | −0.6567 | −0.8997 | 0.9613 | −0.6587 | −0.9877 | 0.0553 | 1.019 | −0.0843 | 0.0338 | −0.1387 | −0.0338 | −0.6837 | −0.2702 | −1.272 | −0.4197 | 0.5623 |
| CENPF | 691312II [symbol] | −1.475 | −0.265 | 1.242 | −0.899 | 0.149 | 0.214 | 1.002 | 0.6615 | −0.531 | −1.073 | 0.087 | −0.31 | −0.1645 | −1.404 | −0.189 | 0.357 |
| CENTG3 | 687661II [symbol] | 0.325 | −0.194 | −0.494 | 0.319 | −0.194 | −0.433 | −0.304 | 0.4435 | 1.246 | 0.487 | 0.274 | −0.167 | −0.163 | 0.146 | 0.288 | −0.109 |
| CES2 | 897031II [symbol] | −0.143 | −0.117 | −0.378 | −0.028 | −0.494 | −0.183 | −0.811 | −0.319 | 0.6115 | 0.039 | 0.106 | −0.613 | −0.6725 | 0.056 | −0.053 | −0.351 |
| CETN3 | 692233II [symbol] | −1.615 | −1.14 | −1.082 | 0.027 | −0.548 | −1.673 | −1.624 | −1.557 | −1.5355 | −0.047 | −0.618 | −0.443 | −1.0385 | −0.7 | −1.293 | −0.566 |
| CGA | 693621II [symbol] | −2.841 | −3.168 | −2.38 | 4.665 | −3.616 | −2.148 | −2.626 | −3.148 | −2.504 | −2.688 | −2.56 | −3.473 | −2.63 | −2.458 | −2.905 | −3.138 |
| CGI-12 | 683023II [symbol] | −0.347 | 0.137 | 0.124 | −0.637 | −0.484 | 0.579 | −0.229 | 0.124 | −0.628 | −0.759 | −0.1 | −0.224 | −0.5485 | −0.491 | −0.489 | −0.102 |
| CGI-45 | 693157II [symbol] | −1.897 | −1.823 | −1.513 | −1.408 | −1.502 | −1.641 | −1.577 | −2.1075 | −2.1685 | −1.002 | −1.325 | −0.693 | −1.2345 | −1.248 | −1.685 | 0.159 |
| CGI-49 | 684734II [symbol] | −0.844 | −0.004 | −0.14 | −0.919 | 0.416 | 0.004 | −0.538 | −0.075 | −0.534 | −0.379 | 0.33 | 1.097 | 0.2505 | −0.213 | −0.289 | 0.136 |
| CGN | 688407II [symbol] | 0.4475 | 0.8515 | −2.539 | −0.0445 | −0.0135 | −1.108 | 0.8395 | −0.117 | −1.382 | 1.164 | −0.0165 | 0.0135 | 0.84 | −0.5515 | 0.1045 | −0.5455 |
| CGNL1 | 690560II [symbol] | 0.6615 | −0.5625 | −0.9345 | 1.654 | 0.2235 | −0.6085 | −0.8295 | −0.973 | 0.6565 | −0.0855 | 0.0076 | 0.3655 | 0.5435 | 0.8445 | 0.6705 | −0.2775 |
| CHAF1B | 682830II [symbol] | −0.12 | −0.327 | 1.68 | −0.27 | −0.645 | 0.54 | 1.55 | −0.486 | −0.0415 | 0.17 | −0.06 | 0.68 | 0.22 | −1.07 | −0.099 | 0.29 |
| CHC1 | 682220II [symbol] | −1.508 | 0.64 | 1.172 | −0.686 | −0.307 | 0.414 | 0.837 | 1.621 | 0.532 | −0.636 | 0.012 | −0.012 | −0.279 | −0.394 | −0.285 | 0.105 |
| CHCHD2 | 681919II [symbol] | −0.3985 | −0.3415 | 0.4665 | 0.0405 | −0.3375 | −0.4235 | 0.4845 | 0.09 | −0.717 | 0.3335 | 0.5945 | −0.0945 | −0.084 | 0.1455 | −0.1115 | 0.1965 |
| CHCHD5 | 686549II [symbol] | −0.5315 | 0.4655 | −0.3275 | 0.2495 | 0.3895 | −0.2235 | −0.7655 | −0.077 | 0.779 | 0.4105 | 0.4835 | 0.3335 | 0.414 | 0.4475 | 0.7395 | −0.0675 |
| CHI3L1 | 689913II [symbol] | −1.583 | −0.004 | 0.545 | 1.372 | −1.423 | −1.109 | 0.814 | −0.4695 | −0.9945 | 0.163 | −0.713 | −0.862 | 0.0085 | 0.004 | −0.03 | 1.146 |
| CHI3L2 | 683767II [symbol] | 0.4135 | −0.8735 | 0.5935 | −0.0855 | 1.118 | −1.313 | 3.175 | −1.603 | 0.51 | 0.3765 | −0.8495 | 1.419 | 1.493 | −0.4465 | −0.9925 | −0.8855 |
| CHKA | 689809II [symbol] | 0.2835 | 0.2005 | −0.1205 | 0.0276 | −0.0155 | −0.0735 | 1.005 | 0.7255 | 0.1325 | −0.4495 | −0.0445 | −0.4865 | −0.259 | −0.4545 | 0.0935 | 0.5695 |
| CHODL | 694845II [symbol] | −2.118 | −1.888 | −0.409 | −2.75 | −3.382 | −1.33 | −1.726 | −0.8325 | −1.782 | −2.872 | −2.079 | −2.918 | −1.6675 | −2.222 | −1.635 | 0.411 |
| CHRDL2 | 686365II [symbol] | −0.004 | −0.247 | −0.105 | −0.493 | 0.373 | 0 | 0.644 | 0.256 | 1.408 | −0.037 | −0.364 | 0.166 | −0.515 | 0.271 | −0.225 | −0.084 |
| CHST2 | 691695II [symbol] | −0.0485 | 0.2345 | −0.0185 | −0.2715 | −0.0695 | 0.2265 | 0.2055 | 0.7645 | 0.552 | −0.2815 | 0.1865 | −0.0845 | 0.1975 | −0.2395 | 0.2085 | −0.0355 |
| CHURC1 | 684314II [symbol] | −1.285 | 0.194 | −0.581 | −0.133 | 0.456 | 0.691 | −0.379 | −0.0965 | 0.5045 | 0.08 | 0.327 | 0.453 | −0.061 | 0.174 | 0.044 | −0.078 |
| CIR | 696941II [symbol] | −0.174 | 0.521 | 0.386 | 0.603 | 0.99 | 0.577 | 0.261 | 0.7605 | 0.654 | 0.832 | 0.089 | 0.688 | 0.1385 | 0.548 | 0.518 | 0.587 |
| CIRBP | 685402II [symbol] | 0.169 | −0.019 | −0.004 | 0.843 | 0.868 | −0.603 | −1.244 | −1.118 | −0.1625 | 0.004 | −0.587 | 0.211 | 0.293 | 0.657 | 0.053 | −0.568 |
| CKLF | 688221II [symbol] | −0.8155 | 0.0185 | 0.7785 | −0.0855 | −0.4825 | −0.1285 | 0.0205 | 0.0725 | −0.0195 | −0.0045 | 0.1835 | 0.0125 | −0.356 | 0.0045 | 0.2755 | −0.1525 |
| CKLFSF6 | 681860II [symbol] | 0.287 | 0.021 | −0.781 | −0.033 | 0.305 | −0.138 | −0.213 | 0.688 | 0.024 | 0.618 | 0.492 | −0.934 | 0.3405 | 0.224 | 0.478 | 0.188 |
| CKLFSF7 | 694344II [symbol] | −1.101 | −0.643 | 0.67 | 0.464 | −0.868 | 0.917 | −0.015 | 0.7355 | −0.576 | 0.276 | 1.752 | −0.781 | −1.24 | −1.089 | −0.415 | 1.303 |
| CKMT1 | 692149II [symbol] | −0.759 | 0.027 | 1.343 | 0.704 | −0.224 | 1.177 | −0.226 | 1.773 | −0.1725 | 0.817 | −1.193 | 0.513 | 1.7515 | 2.334 | 0.741 | 1.572 |
| CKS1B | 696332II [symbol] | −1.944 | −1.296 | −0.853 | −1.685 | −1.3 | −1.635 | −1.511 | −1.064 | −2.3 | −1.84 | −1.165 | −0.949 | −1.462 | −2.443 | −1.228 | −0.988 |
| CKS2 | 691352II [symbol] | −2.668 | 0.029 | 0.997 | −0.029 | −0.301 | −0.487 | 0.293 | −0.345 | −0.6495 | 0.614 | 0.804 | −0.937 | 0.379 | −0.86 | −0.763 | −0.326 |
| CL640 | 695106II [symbol] | −0.622 | −0.431 | 0.297 | −0.671 | −1.184 | −1.008 | −0.255 | −1.0715 | −1.0785 | −0.756 | −0.345 | −0.791 | −0.909 | −1.033 | −1.34 | −0.185 |
| CLDN23 | 683062II [symbol] | −0.38 | −0.668 | −0.576 | −1.177 | −0.997 | −0.202 | 1.72 | 0.4125 | 0.172 | 0.931 | 0.304 | −0.268 | −0.985 | −1.423 | 0 | 0.467 |
| CLDN3 | 686923II [symbol] | −2.056 | −0.1765 | −0.4345 | −1.503 | −0.9145 | 0.4815 | 1.088 | 0.7465 | 0.378 | 0.3295 | −0.9115 | 0.4095 | −0.0245 | −0.8425 | 0.8975 | 1.223 |
| CLDN4 | 681712II [symbol] | −0.4535 | 0.4105 | −0.6325 | −0.5075 | −1.53 | −0.0065 | 0.5815 | 0.6455 | −1.006 | 0.2525 | 0.5775 | 0.5345 | 0.141 | −2.337 | 0.9115 | 0.9765 |
| CLDN8 | 695858II [symbol] | 0.139 | 0.964 | 0.544 | 0.698 | −0.17 | −0.012 | 2.017 | 0.8345 | 0.361 | 0.653 | 0.779 | 0.587 | 0.217 | 0.017 | −0.232 | 1.499 |
| CLGN | 691474II [symbol] | −1.766 | −0.3925 | 1.277 | −0.8075 | 0.7105 | 1.045 | 0.5705 | 0.626 | −0.1935 | 0.2105 | 0.8265 | 0.0325 | 1.25 | −1.801 | −0.3995 | 3.383 |
| CLIC6 | 692133II [symbol] | −0.963 | 1.423 | −1.11 | −0.979 | −0.23 | 0.074 | −0.729 | −0.004 | 1.901 | 2.901 | −0.13 | 4.722 | −0.437 | −0.679 | 2.046 | 0 |
| CLNS1A | 693088II [symbol] | 1.06 | −0.623 | −1.06 | −0.651 | −0.771 | −1.076 | −1.227 | −1.458 | −1.0955 | −0.933 | −0.752 | −1.074 | −0.924 | −0.711 | −1.408 | −0.96 |
| CLTC | 694429II [symbol] | −0.263 | −0.291 | −0.373 | −0.651 | −0.645 | 1.353 | −0.257 | −0.773 | −1.106 | 0.726 | −0.443 | −0.267 | −0.713 | −0.522 | −0.371 | 0.918 |
| CLU | 691706II [symbol] | 0.8665 | 0.5265 | −0.4715 | 0.5475 | 0.0515 | −1.166 | 2.588 | −1.746 | −0.0055 | −0.4735 | −0.8475 | −0.2895 | −0.7635 | 0.8475 | 0.0055 | 0.0855 |
| CMAS | 682207II [symbol] | −0.427 | −0.265 | 0.751 | −0.05 | −0.115 | 0.011 | 0.713 | −0.6065 | −1.337 | 0.371 | −0.007 | −0.275 | −0.0625 | 0.007 | 0.493 | −0.336 |
| CML66 | 682090II [symbol] | 0.09 | −0.313 | 0.842 | −0.22 | −0.75 | −0.1 | −0.04 | −0.404 | −0.869 | 0.04 | 0.49 | 0.06 | −0.26 | −0.53 | −1.067 | 0.7 |
| CNIH | 685679II [symbol] | −0.8955 | −0.6635 | −0.7375 | 0.0145 | 0.4305 | −0.3495 | −1.142 | −1.403 | −0.967 | 0.4515 | 0.1465 | 0.8305 | −0.748 | 0.1715 | −0.2935 | −0.0145 |
| CNOT10 | 688504II [symbol] | −0.05 | −0.206 | −0.186 | 0.03 | 0.119 | 0.327 | 0.315 | −0.0045 | −0.1705 | 0.174 | −0.216 | 0.16 | −0.004 | −0.036 | −0.127 | 0.085 |
| CNOT2 | 688501II [symbol] | −0.1155 | 0.0925 | −0.2075 | −0.4315 | 0.3715 | 0.4225 | 0.7645 | 0.0795 | 0.55 | 0.0735 | 0.7905 | −0.0585 | −0.123 | −0.1885 | 0.3245 | 0.0805 |
| CNOT7 | 687730II [symbol] | −0.2205 | −0.0485 | −0.8155 | 0.1245 | 0.3815 | −0.1125 | −0.0645 | −0.2125 | −0.2335 | 0.3965 | 0.0855 | 0.4175 | 0.2985 | 0.3025 | −0.0575 | −0.4655 |
| CNP | 684683II [symbol] | −1.885 | −1.073 | −0.542 | −0.719 | −1.563 | −0.743 | −0.849 | −0.0315 | −0.4945 | −1.98 | −1.584 | −1.043 | −0.747 | −0.184 | −1.598 | −1.38 |
| CNTNAP2 | 687862II [symbol] | −0.221 | −0.471 | 0.732 | −1.999 | 0.092 | −1.433 | −0.999 | 1.262 | 0.6345 | 2.715 | 1.985 | 2.699 | −1.359 | −2.161 | 1.373 | 3.097 |
| COASY | 694546II [symbol] | −0.416 | −0.347 | 0.375 | 0.31 | −0.28 | −0.926 | −0.322 | −0.286 | 0.1505 | −0.116 | −0.376 | −0.008 | 0.204 | 0.289 | 0.149 | −0.501 |
| COL11A2 | 698658II [symbol] | −0.075 | −0.192 | −0.339 | −0.11 | −0.192 | −0.967 | 0.47 | −0.3375 | −0.1235 | 0.503 | −0.656 | −0.57 | −0.3965 | −0.482 | −0.199 | 0.197 |
| COL27A1 | 690790II [symbol] | −0.0235 | 0.3315 | −0.0985 | −1.199 | −0.0395 | −0.1415 | 1.136 | 0.1985 | −0.6845 | 1.316 | −0.5405 | 0.6675 | 0.4905 | −1.341 | 0.2325 | −1.145 |
| COL4A5 | 695636II [symbol] | 0.332 | 0.253 | −1.356 | −1.295 | 0.885 | −1.173 | −0.005 | −1.8235 | 0.8815 | −0.167 | 1.391 | 0.801 | 1.849 | −0.028 | 0.458 | −0.791 |
| COL9A1 | 688215II [symbol] | −0.248 | −0.71 | 0.156 | −0.754 | −0.374 | 0.929 | 1.542 | 0.402 | −0.5485 | −0.756 | 0.575 | −0.461 | −1.222 | 1.266 | −0.43 | 0.977 |
| COL9A3 | 694474II [symbol] | −2.056 | −2.294 | −3.618 | −2.774 | −2.471 | −2.602 | 0.515 | −0.705 | −2.465 | −2.71 | −1.795 | −0.888 | −2.807 | −2.749 | −1.43 | −3.057 |
| CDMMD [symbol] | 685920II [symbol] | −0.371 | −0.577 | −0.344 | −0.828 | −0.64 | −0.781 | −0.433 | −0.661 | −1.468 | −0.543 | −0.131 | −0.285 | −0.6175 | −0.906 | −0.796 | −0.155 |
| COMMD5 | 683292II [symbol] | −0.4995 | 0.2195 | 0.3055 | −0.0815 | 0.0135 | −0.7045 | 0.2525 | 0.447 | −0.4235 | −0.2775 | −0.2435 | −0.3486 | −0.0085 | −0.1095 | −0.1805 | 0.1025 |
| COPS7B | 686043II [symbol] | 0.582 | −0.072 | 0.339 | 0.016 | −0.026 | −0.203 | −0.143 | −0.055 | 0.3025 | −0.408 | −0.217 | 0.024 | 0.205 | −0.223 | −0.052 | −0.016 |
| CORO6 | 691905II [symbol] | −0.0355 | 0.3895 | −0.1355 | 0.2285 | 1.909 | 0.0405 | −0.1015 | 0.9185 | 1.054 | −0.1985 | 2.384 | −0.6605 | 2.118 | −0.4985 | 0.7105 | −0.5385 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| CORT | 688125II [symbol] | −0.7105 | 0.0325 | 2.138 | −0.2635 | 0.1815 | 0.1115 | −0.0285 | 0.1295 | 0.479 | 0.1065 | −0.8395 | −0.2245 | −0.33 | 0.4485 | −0.2455 | −0.4065 |
| COX17 | 698533II [symbol] | −0.656 | 0.275 | 0.711 | 1.304 | 0.394 | 0.008 | 0.49 | 0.3355 | 0.9745 | 1.246 | 0.715 | −0.095 | −0.1825 | 1.083 | −0.042 | 1.54 |
| COX5A | 690776II [symbol] | −0.979 | −0.275 | −0.391 | 0.112 | −0.421 | −0.251 | 0.198 | −0.0495 | −1.115 | 0.03 | 0.33 | −0.28 | −0.6325 | 0.156 | −0.691 | 0.349 |
| COX5A | 693143II [symbol] | −2.086 | −0.909 | −0.793 | −0.715 | −1.325 | −1.157 | −0.882 | −0.8455 | −2.057 | −1.07 | −0.738 | −1.265 | −1.4645 | −0.863 | −1.657 | −0.645 |
| COX6A1 | 690654II [symbol] | −0.9205 | 0.1665 | 0.3495 | 0.2755 | 0.5945 | −0.6965 | −0.2725 | −0.2335 | −0.069 | 0.6495 | 0.0545 | −0.5445 | 0.4175 | −0.1005 | −0.0785 | 0.2295 |
| COX6C | 685569II [symbol] | −1.808 | 1.316 | −1.277 | 3.487 | 1.651 | −1.392 | −1.262 | −0.3855 | 0.892 | 3.457 | 2.557 | 0.3555 | −0.439 | 3.391 | 0.6465 | −0.8235 |
| COX7A2 | 697312II [symbol] | −1.334 | −1.406 | −0.014 | −0.037 | 0.127 | −0.284 | −0.18 | −0.0785 | 0.5865 | 0.826 | −0.201 | −0.664 | −1.62 | 0.156 | −0.404 | 0.603 |
| CP | 683747II [symbol] | −0.6765 | −0.6375 | 0.0925 | 0.6225 | 0.0265 | −0.0265 | −0.0415 | −0.484 | 0.0475 | −0.8445 | −0.8685 | −0.5285 | −0.6125 | 0.3015 | −1.02 | −0.2575 |
| CPAMD8 | 690973II [symbol] | −0.126 | −0.375 | 0.625 | −0.037 | 0.206 | −0.265 | −0.142 | 0.576 | 0.037 | −1.056 | −0.061 | 3.166 | 3.845 | 2.207 | 0.499 | 2.051 |
| CPB1 | 697093II [symbol] | 0.513 | 0.429 | −1.003 | 0.617 | 0.661 | −0.612 | −0.524 | −0.342 | 0.4035 | 1.022 | 0.133 | 2.114 | 0.3125 | −0.666 | 1.012 | −0.678 |
| CPEB3 | 683341II [symbol] | −0.3475 | 0.0645 | −0.7445 | 0.2985 | 0.3075 | −0.2725 | −0.5415 | −1.118 | −0.0995 | 0.8425 | 0.0875 | 0.8565 | −0.0005 | 0.6465 | −0.0405 | 0.5945 |
| CPEB4 | 681384II [symbol] | −0.088 | −0.011 | 1.206 | −0.446 | −1.505 | −0.133 | 0.011 | −0.729 | −0.349 | −0.802 | −1.932 | 1.172 | 0.026 | −1.156 | 0.14 | 0.13 |
| CPSF3 | 690199II [symbol] | 0.694 | 0.838 | −0.056 | 2.285 | 1.011 | 0.281 | 0.521 | 0.1565 | 1.3865 | 1.124 | 1.62 | 0.988 | 1.16 | 1.591 | 1.24 | 0.204 |
| CRABP1 | 694973II [symbol] | 0.255 | 0.568 | 0.993 | 0.473 | 0.032 | 0.401 | −0.018 | −0.183 | −0.097 | −0.362 | −0.089 | −0.163 | −0.2425 | 0.757 | −0.035 | −0.376 |
| CREB3L4 | 690183II [symbol] | −0.94 | −1.411 | −2.371 | −1.61 | −1.891 | −1.9 | −1.08 | −1.715 | −2.167 | −1.4 | −0.94 | −1.84 | −1.876 | −2.15 | −2.257 | −1.88 |
| CRIP1 | 686560II [symbol] | −1.201 | 0.496 | 0.653 | 0.043 | 0.631 | −0.304 | −2.408 | 0.913 | 1.556 | −0.812 | 0.292 | 0.097 | −0.6455 | 0.141 | 0.916 | 0.216 |
| CRYBB2 | 683857II [symbol] | 0.939 | 0.013 | 1.595 | 0.844 | −0.253 | 1.605 | 0.086 | 0.5425 | 1.017 | −0.512 | −0.544 | −0.438 | 0.0565 | 0.928 | −0.36 | −0.425 |
| CRYL1 | 692598II [symbol] | 0.062 | 0.103 | −0.353 | 0.589 | 0.232 | −0.471 | −0.88 | −0.608 | 0.076 | 0.485 | −0.175 | −0.395 | −0.1375 | 0.986 | 0.067 | 0.011 |
| CRYZ | 683831II [symbol] | 0.3215 | 0.4525 | 0.7585 | −0.7725 | 1.166 | 0.7095 | −1.169 | −0.69 | −0.4945 | 0.1365 | 0.5695 | 0.7495 | −0.273 | −0.6155 | −1.032 | −0.0005 |
| CSDA | 681102II [symbol] | −0.8075 | 0.6285 | 0.8245 | −0.7915 | −0.6265 | −0.0985 | 0.4945 | 0.758 | −0.191 | −0.3835 | 0.3295 | 0.1655 | 0.079 | −0.5975 | 0.4115 | 0.0315 |
| CSE1L | 693508II [symbol] | −2.161 | −0.933 | −2.377 | −2.035 | −1.895 | −1.684 | −1.129 | −1.147 | −2.0965 | −1.943 | −0.625 | −1.715 | −1.2515 | −1.714 | −2.156 | −1.614 |
| CSK | 693320II [symbol] | 0.314 | 0.249 | 0.449 | 0.205 | 0.262 | 0.805 | 0.423 | 0.5835 | 0.498 | 0.064 | 0.252 | 0.079 | 0.336 | 0.241 | 0.512 | 0.306 |
| CSRP1 | 681588II [symbol] | 0.021 | −0.001 | −0.299 | −0.041 | 0.004 | −0.056 | −1.452 | 0.001 | −0.9505 | −0.092 | 0.219 | 0.417 | −0.7305 | 0.321 | 0.289 | −0.053 |
| CSRP2 | 691158II [symbol] | 0.8765 | −0.0745 | 2.228 | −0.3665 | −0.4325 | 2.179 | 1.36 | 1.144 | −0.4835 | −0.7195 | −0.2045 | −0.6855 | −1.075 | 0.0745 | −0.1165 | −0.2645 |
| CST6 | 688329II [symbol] | −0.97 | 2.042 | −0.417 | −1.445 | 1.698 | 0.677 | 1.442 | 1.213 | −1.604 | −0.622 | 0.562 | 1.054 | −0.824 | 0.266 | 0.103 | 0.987 |
| CSTB | 688630II [symbol] | −1.191 | −0.4695 | 1.666 | −0.3155 | 0.4005 | −0.5035 | 2.018 | 0.3585 | −0.244 | −0.3235 | −0.0455 | 0.6175 | −0.9545 | −0.0055 | −0.1515 | 0.0055 |
| CTAG1B | 685110II [symbol] | −0.735 | −0.326 | 0.042 | −0.549 | 0.142 | 0 | −0.402 | 2.11 | 0.623 | 0.456 | −0.957 | −1.104 | 0.046 | −0.13 | 0.638 | −0.795 |
| CTBP2 | 690880II [symbol] | 0.036 | 0.367 | −0.445 | −0.5 | 0.34 | 0.426 | −0.465 | 0.1375 | 0.1085 | −0.012 | 0.283 | 0.193 | −0.12 | 0.107 | 0.538 | 0.053 |
| CTNNA1 | 682257II [symbol] | −0.242 | −0.025 | 0.14 | −0.181 | −0.082 | 0.352 | −0.417 | −0.507 | −0.7735 | −0.431 | 0.542 | 0.595 | 0.3225 | −0.253 | 0.035 | 0.463 |
| CTNNAL [symbol] | 683652II [symbol] | 1.023 | 0.5355 | 0.7555 | 1.022 | −0.2545 | −0.2615 | −0.2265 | −0.572 | −0.0795 | 0.8225 | 0.3425 | −0.4195 | −0.345 | −0.2395 | −0.0635 | 0.4855 |
| CTNND2 | 692813II [symbol] | −0.231 | 1.076 | 0.557 | 0.243 | −0.462 | −0.342 | −0.06 | −0.3595 | −0.1565 | 1.142 | 0.584 | 0.12 | 0.73 | −1.049 | 0.173 | 0.303 |
| CTPS | 688832II [symbol] | 0.135 | −0.128 | 0.062 | −0.173 | −0.194 | 0.725 | 0.21 | −0.062 | 0.1855 | 0.303 | 0.375 | −0.458 | −0.5405 | −0.74 | −0.172 | 0.31 |
| CTSL2 | 696809II [symbol] | −3.2 | −1.437 | −1.564 | −3.349 | −3.181 | −1.972 | −0.835 | −1.4935 | −3.094 | −2.976 | −1.861 | −2.901 | −2.4545 | −3.426 | −3.012 | −2.723 |
| CTSS | 688045II [symbol] | −0.3315 | −0.6815 | 0.7055 | 0.3775 | 0.5025 | 0.3285 | 0.6695 | −0.7335 | −0.934 | −0.1175 | 0.4065 | −0.5725 | 0.077 | −0.3895 | −0.6305 | 0.7905 |
| CTTN | 690610II [symbol] | −1.348 | 0.466 | −0.215 | −0.481 | 0.144 | −0.155 | 0.661 | 0.707 | 0.5855 | −0.156 | 0.514 | −0.152 | −0.4775 | −0.281 | 0 | 0.004 |
| CX3CL1 | 762475II [symbol] | 3.59 | 1.57 | 3.48 | 1.658 | −0.03 | 2.156 | 4.336 | 4.5675 | 1.655 | 1.058 | 0.68 | 3.476 | 0.349 | 1.503 | 1.11 | 3.476 |
| CXCL9 | 694763II [symbol] | 3.031 | 4.374 | 2.939 | 4.879 | 2.024 | 5.016 | 3.599 | 3.6495 | 1.4145 | 4.887 | 4.927 | 2.821 | 1.9125 | 2.516 | 1.812 | 4.807 |
| CXorf39 | 682198II [symbol] | −0.9845 | 0.6755 | 0.2135 | 0.1195 | 0.0825 | −0.4945 | 0.5975 | −1.173 | −0.938 | −0.0945 | 0.4935 | −0.1235 | 0.446 | −0.1645 | −0.2605 | 0.2915 |
| CXXC4 | 684240II [symbol] | −0.0145 | 0.3535 | 0.4175 | 0.7485 | −2.215 | −1.551 | −0.3475 | −0.1335 | −0.1315 | 1.571 | 0.8435 | −2.266 | 0.633 | 0.7775 | 0.0745 | 0.4285 |
| CYB5 | 690446II [symbol] | −0.669 | 0.875 | 0.444 | −0.056 | 0.911 | −0.03 | −1.765 | −0.111 | −0.84 | 0.03 | 1.335 | 1.184 | 1.336 | 0.045 | −0.833 | −0.183 |
| CYB561 | 683867II [symbol] | −0.025 | −0.356 | 0.133 | −0.58 | 0.617 | 2.52 | 0.804 | 0.7995 | −0.8505 | 1.443 | −0.047 | 1.391 | 0.1885 | −0.509 | 0.633 | 1.264 |
| CYC1 | 683057II [symbol] | −0.8785 | −0.6485 | 1.237 | 0.3355 | −0.6355 | 1.257 | 0.9745 | 0.8255 | −0.3545 | 0.2745 | −0.2975 | −1.573 | −2.481 | 0.0925 | −1.247 | 0.1165 |
| CYHR1 | 697209II [symbol] | 0.818 | 1.527 | 0.295 | 1.426 | 1.088 | 0.299 | 1.069 | 0.7955 | 0.969 | 1.073 | 0.908 | 1.317 | 1.025 | 1.163 | 1.168 | 0.89 |
| CYP2A13 | 689236II [symbol] | −0.222 | 0.095 | 0.022 | 1.934 | −0.263 | 0.293 | 0.095 | 0.244 | 1.645 | 2.79 | −0.022 | −0.299 | −0.142 | 1.147 | −0.022 | −0.155 |
| CYP4X1 | 681940II [symbol] | −1.11 | 0.3313 | −1.267 | −0.6697 | 3.196 | 2.319 | 0.0393 | −0.0393 | 2.466 | 1.272 | −0.9837 | −1.017 | 1.685 | 1.483 | −0.5567 | 1.053 |
| D15Wsu7 [symbol] | 684516II [symbol] | −1.228 | 0.126 | −0.261 | 0.263 | 1.406 | 0.18 | −0.288 | −0.512 | 4.215 | 0.574 | 3.249 | 0.046 | −0.351 | 3.129 | 3.895 | −0.066 |
| DACH1 | 692995II [symbol] | −0.7635 | 0.1305 | −0.5195 | 0.6695 | 0.3735 | 0.0835 | −0.2945 | −0.8105 | −0.3995 | 0.3475 | 0.4925 | 0.5405 | 0.323 | −0.0865 | 0.0805 | −0.1985 |
| DACH1 | 694269II [symbol] | 0.347 | 1.152 | 0.335 | 1.065 | 1.348 | 0.918 | 0.206 | 0.4585 | 0.86 | 3.522 | 3.21 | 0.827 | 0.8895 | 3.562 | 1.232 | 0.895 |
| DACH1 | 697247II [symbol] | 0.509 | 2.275 | −0.374 | 2.641 | 1.981 | 0.162 | −0.496 | −0.1035 | 1.12 | 3.573 | 3.92 | 1.082 | 1.493 | 4.227 | 2.011 | 1.061 |
| DAP3 | 689542II [symbol] | 0.443 | 0.533 | −0.039 | 0.848 | 1.358 | 0.07 | 0.342 | −0.2825 | 0.3355 | 0.612 | 0.401 | 1.016 | 0.42 | 0.506 | 0.76 | 0.468 |
| DAPP1 | 681676II [symbol] | −1.435 | −1.233 | −0.539 | −1.278 | −1.299 | −0.908 | −0.257 | −1.5035 | −1.277 | −1.137 | −1.427 | −0.963 | −0.841 | −1.698 | −0.807 | −1.22 |
| DAZAP2 | 686305II [symbol] | 0.7095 | 0.2455 | −0.4785 | 0.3465 | 0.2675 | −0.2005 | −0.7305 | −0.157 | −0.5555 | 0.3575 | 0.2275 | 0.0895 | 0.678 | 0.1155 | 0.1035 | 0.3295 |
| DBI | 687444II [symbol] | −0.4475 | 0.3055 | 0.2215 | 0.2935 | −0.8865 | −0.1045 | −0.6905 | 0.0095 | −0.37 | −0.2835 | 0.6075 | 0.4085 | 0.266 | 0.0265 | −0.1945 | 0.4465 |
| DBN1 | 685238II [symbol] | 0.618 | −0.148 | −0.007 | −0.857 | −0.568 | 0.539 | 0.594 | 0.783 | −0.743 | −1.48 | 0.268 | −0.67 | −0.6295 | −0.148 | 0.007 | 0.145 |
| DC13 | 693725II [symbol] | −2.009 | −1.634 | −0.682 | −0.772 | −1.654 | −1.643 | −1.341 | −1.443 | −2.2565 | −1.519 | −1.339 | −2.052 | −2.1565 | −1.371 | −1.627 | −1.402 |
| DCPS | 683983II [symbol] | −0.566 | −0.306 | 0.191 | −0.77 | −0.165 | 0.459 | 0.936 | 0.066 | −0.3445 | −0.213 | 0.565 | 0.007 | −0.2345 | −0.27 | −0.19 | −0.048 |
| DDHD2 | 683946II [symbol] | 0.1645 | −0.1035 | −0.8185 | −0.2495 | −0.2945 | −0.4305 | 0.3925 | −0.54 | −0.08 | −0.2465 | 0.6835 | 0.0645 | −0.2905 | 0.3295 | −0.2085 | −0.5595 |
| DDX17 | 694961II [symbol] | 0.08 | −0.376 | −0.347 | −0.615 | 0.169 | −0.392 | −0.532 | −0.858 | −0.0355 | −0.086 | 0.32 | 0.03 | −0.029 | −0.366 | −0.361 | −0.225 |
| DDX39 | 681363II [symbol] | −0.4825 | 0.1255 | 0.9465 | −0.1195 | −0.5755 | 0.0985 | 0.7935 | 0.4545 | −0.841 | −0.0865 | −0.6215 | −0.8585 | −0.2395 | −0.7215 | 0.3275 | 0.0325 |
| DDX48 | 696985II [symbol] | −1.526 | −0.675 | −0.25 | −0.956 | −0.66 | −0.318 | 0.142 | −1.1185 | −1.4035 | −0.186 | 0.121 | −0.614 | −0.439 | −0.879 | −0.202 | −1.174 |
| DDX58 | 691620II [symbol] | −0.447 | 0.002 | 0.927 | 0.163 | −0.009 | 0.935 | 1.026 | 1.768 | −0.002 | 0.78 | −0.109 | −1.106 | −1.255 | −0.975 | −0.311 | 0.534 |
| DEF6 | 682749II [symbol] | 0.026 | −0.134 | 0.177 | −0.08 | 0.104 | 0.69 | 0.363 | 0.631 | 0.6695 | 0.104 | −0.375 | −0.767 | −0.2735 | −0.336 | 0.265 | 0.326 |
| DEK | 688515II [symbol] | −1.49 | 0.421 | 1.85 | −0.775 | −0.496 | −0.204 | 0.535 | 0.5575 | −0.324 | 0.004 | 0.355 | −0.332 | −0.1915 | −0.63 | −0.838 | 0.945 |
| DHCR7 | 694109II [symbol] | −0.5445 | −0.6005 | 0.0825 | 1.157 | 0.9055 | −0.0155 | −1.147 | −0.45 | −1.163 | −0.6995 | 0.3945 | 0.4795 | −0.126 | 1.452 | −0.5615 | 0.4185 |
| DHRS7 | 682438II [symbol] | −0.647 | 0.126 | −1.295 | −0.221 | 0.001 | −0.631 | −0.848 | −1.464 | −0.7505 | 0.68 | 0.409 | 0.002 | 0.053 | 0.463 | −0.699 | 0.021 |
| DHX57 | 686263II [symbol] | −1.104 | −0.311 | −0.211 | 0.184 | 0.105 | 0.004 | 0.327 | 0.6665 | −0.0925 | 0.347 | 0.394 | −0.358 | −0.701 | 0.013 | −0.032 | 0.246 |
| DIRC1 | 691337II [symbol] | −1.418 | −1.283 | −0.061 | −0.074 | 0.724 | −0.051 | 0.236 | −0.11 | −0.166 | 0.237 | 0.196 | 0.447 | 0.5475 | 0.63 | 0.051 | −0.78 |
| dJ222E13 [symbol] | 689427II [symbol] | −1.49 | −0.733 | 2.464 | 2.705 | −0.05 | 0.594 | −1.422 | 1.93 | 1.581 | −1.129 | −1.055 | 0.601 | −0.877 | 0.05 | −1.69 | 1.95 |
| DKFZP43 [symbol] | 689930II [symbol] | −0.359 | −0.085 | 0.751 | 0.299 | 0.006 | −0.148 | −0.383 | 0.1065 | 0.82 | 0.454 | −0.191 | 0.083 | −0.5205 | 0.851 | 0.527 | −0.326 |
| DKFZp43 [symbol] | 689808II [symbol] | −0.775 | −0.347 | 0.558 | −0.332 | 0.601 | 0.392 | 1.688 | 0.6495 | −0.214 | −0.526 | −1.322 | 0.803 | −0.4305 | 0.073 | −0.777 | −0.27 |
| DKFZP43 [symbol] | 681071II [symbol] | −0.5155 | 0.3115 | 0.8255 | −0.6115 | −0.7485 | 0.8715 | 1.83 | 2.396 | −0.103 | −0.2025 | −0.1655 | −0.7375 | −0.6105 | 0.0185 | −0.5475 | 1.703 |
| DKFZp43 [symbol] | 683495II [symbol] | −0.0155 | 0.0345 | 0.8745 | −0.1525 | −0.4985 | −0.0025 | 0.5685 | −0.1335 | 0.0235 | −0.3415 | −0.5025 | −0.5805 | 0.0345 | −0.9515 | 0.0805 | −0.1775 |
| DKFZP56 [symbol] | 684976II [symbol] | 0.648 | 0.51 | −0.92 | −0.509 | −0.304 | −0.779 | 2.51 | −0.3615 | −0.306 | −0.729 | 0.319 | −1.121 | 0.4805 | −0.318 | 0.488 | 0.441 |
| DKFZp56 [symbol] | 697724II [symbol] | 0.373 | 0.81 | 0.603 | 0.001 | 0.436 | −0.103 | −0.45 | 0.576 | 0.3095 | 0.401 | 0.792 | 0.304 | 0.143 | 0.199 | 0.305 | 0.352 |
| DKFZP56 [symbol] | 683868II [symbol] | 0.281 | −0.241 | 0.584 | −0.377 | −0.455 | 1.603 | −0.209 | −0.111 | −0.536 | 0.235 | −0.28 | −0.022 | 0.138 | 0.342 | 0.022 | −0.283 |
| DKFZP56 [symbol] | 688875II [symbol] | −0.6255 | 0.2795 | 0.0575 | −0.8785 | −0.9395 | −0.4185 | −0.3465 | 0.516 | −0.8285 | −0.2145 | 0.0195 | −0.2425 | −0.7 | 0.5915 | −0.9865 | 0.6805 |
| DKFZP56 [symbol] | 682674II [symbol] | −0.741 | 0.162 | −0.494 | −0.064 | 0.088 | −0.212 | −0.56 | −0.2155 | −0.0805 | 0.377 | 0.631 | 0.356 | 0.228 | 0.644 | 0.07 | −0.167 |
| DKFZp58 [symbol] | 695204II [symbol] | −1.53 | −1.555 | −1.542 | −0.621 | −0.551 | −0.848 | −1.642 | −1.2305 | −1.556 | −1.41 | −0.198 | −0.558 | −1.2245 | −0.561 | −1.397 | −0.617 |
| DKFZp76 [symbol] | 681865II [symbol] | −0.3817 | 0.2193 | −0.0598 | 0.7583 | −0.2437 | −0.5037 | −0.4637 | −0.0168 | 0.0168 | 2.979 | 0.2413 | −0.3307 | 0.0343 | 1.292 | 0.4293 | 0.2843 |
| DLK1 | 685295II [symbol] | 0.364 | −0.007 | 0.842 | 0.251 | −0.161 | 1.286 | 3.768 | 0.257 | 0.895 | −0.455 | −0.51 | −0.234 | −0.7685 | −0.095 | 0.534 | 0.333 |
| DLX5 | 682457II [symbol] | −1.293 | 3.158 | −1.831 | −2.199 | −0.104 | 1.879 | −2.824 | −0.933 | 0.94 | −0.68 | 0.597 | 2.23 | 3.848 | 0.878 | 3.181 | 1.222 |
| DNAJA4 | 681226II [symbol] | −1.109 | 0.14 | 0.091 | −0.095 | 0.458 | −0.885 | −0.997 | 0.03 | −0.825 | 0.5 | −0.504 | −0.03 | −1.84 | 0.165 | −0.894 | 0.477 |
| DNAJC1 | 692749II [symbol] | −0.037 | 2.128 | 1.559 | 2.015 | 1.479 | 1.934 | 2.289 | 1.196 | 1.6525 | 0.432 | 2.093 | 0.336 | 1.7065 | 0.867 | 1.676 | 0.523 |
| DNAJC12 | 696427II [symbol] | −0.726 | 0.374 | −0.103 | 1.514 | 2.607 | −1.814 | −1.759 | −2.4005 | −0.3875 | 3.315 | 1.402 | 141 | 1.1265 | 3.707 | 0.326 | −0.159 |
| DNAJD1 | 690353II [symbol] | −1.062 | −1.588 | −1.186 | −0.0415 | 0.4355 | 0.9265 | −0.9185 | 0.0855 | −0.2225 | −0.1545 | 0.8615 | 0.1955 | −0.63 | 0.4755 | 0.0205 | −0.7585 |
| DNALI1 | 695070II [symbol] | 2.329 | 4.175 | −0.085 | 3.722 | 4.258 | 2.916 | −0.243 | 0.9765 | 4.471 | 3.635 | 0.935 | 4.066 | 2.8575 | 3.916 | 3.089 | 3.384 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| DNMT1 | 681126II [symbol] | −0.3683 | −0.0693 | 0.3027 | 0.1957 | 0.0508 | 0.3197 | −0.2283 | 0.1967 | 0.5467 | −0.8143 | −0.7223 | −0.3103 | −0.0188 | 0.0528 | 0.1477 | 0.0298 |
| DQX1 | 697175II [symbol] | −0.242 | −1.095 | 0.207 | −0.814 | −0.848 | −0.511 | −0.847 | −0.9885 | −0.171 | 0.412 | −0.989 | −0.203 | −0.6855 | −1.383 | −0.298 | 0.774 |
| DSCR1 | 690367II [symbol] | 0.884 | 0 | 1.087 | −0.574 | −0.331 | 0.444 | 1.188 | −0.6655 | −0.947 | −0.745 | −0.12 | 0.09 | −1.101 | −0.395 | 0.612 | 1.084 |
| DSCR1L2 | 685798II [symbol] | −0.174 | 0.244 | −0.139 | −0.8 | 0.287 | −0.581 | 0.848 | −0.279 | 0.0655 | 0.212 | −0.444 | 0.262 | 0.36 | −0.023 | −0.347 | 0.072 |
| DSP | 695661II [symbol] | −0.533 | 0.379 | −1.118 | −0.806 | 0.386 | −0.852 | −2.051 | −2.332 | 2.054 | 0.459 | −0.134 | 0.732 | 0.033 | −0.91 | 0.15 | −1.301 |
| DTYMK | 692933II [symbol] | −1.518 | −0.687 | −0.362 | −1.34 | −1.073 | −1.11 | −1.236 | −0.6395 | −1.5155 | −1.527 | −1.132 | −0.976 | −1.3305 | −1.373 | −1.127 | −0.758 |
| DUSP23 | 691997II [symbol] | −0.3365 | −0.1285 | −0.7975 | 0.0945 | 0.3355 | −0.0005 | −0.0565 | 0.7685 | 0.768 | −0.4105 | −1.069 | 0.3005 | 0.2855 | −0.4655 | 0.1145 | 0.0895 |
| DUSP4 | 693976II [symbol] | −0.23 | 1.21 | −0.049 | 1.229 | 0.372 | 1.721 | 0.396 | 0.227 | 1.8675 | 2.167 | 0.209 | 0.137 | 0.569 | 2.105 | 0.047 | 0.495 |
| DUSP6 | 693200II [symbol] | 1.258 | 0.524 | −0.74 | −0.471 | 0.197 | 1.259 | −0.25 | −0.2305 | −0.072 | 0.858 | −0.513 | 0.925 | 1.031 | 0.65 | −0.103 | 1.046 |
| DVL1 | 682613II [symbol] | −0.706 | −0.106 | 0.022 | 0.466 | 0.06 | −0.415 | −0.592 | 0.185 | 0.554 | 0.128 | −0.872 | −0.489 | −0.7335 | 0.295 | 0.084 | −0.82 |
| DXYS155 [symbol] | 694208II [symbol] | −0.076 | 0.053 | 0.151 | 0.482 | 0.384 | 0.117 | 0.018 | 0.5805 | 0.5985 | 0.281 | −0.058 | −0.145 | 0.0545 | 0.069 | 0.028 | 0.416 |
| DYRK4 | 697043II [symbol] | 0.275 | 0.648 | −0.146 | 0.277 | 0.287 | 1.061 | 1.083 | 0.4005 | 0.517 | 0.447 | 0.314 | 0.459 | 0.735 | 0.405 | 0.53 | 0.454 |
| [symbol] 2 | 695983II [symbol] | −1.337 | −0.979 | 0.534 | −1.028 | −1.246 | −0.737 | 0.189 | −0.592 | −0.914 | −0.759 | −0.29 | −0.687 | −0.9445 | −0.692 | −1.083 | 0.082 |
| EAF2 | 690585II [symbol] | 0.078 | 0.775 | −0.068 | 0.147 | 0.436 | −0.842 | −0.425 | −0.1185 | 0.385 | 0.008 | −0.707 | 0.536 | 0.141 | 0.096 | −0.01 | −0.528 |
| EAP30 | 695304II [symbol] | −0.259 | −0.558 | −0.026 | −0.246 | −0.628 | −0.522 | −0.76 | −0.114 | −0.405 | −0.824 | −0.86 | −0.246 | −0.212 | −0.317 | −0.265 | −0.323 |
| ECHDC1 | 695068II [symbol] | 0.109 | −1.354 | −1.189 | 0.477 | −0.197 | 1.442 | 0.47 | 0.0415 | −1.604 | 0.501 | −0.714 | −1.542 | −1.5885 | −0.076 | −0.635 | 0.01 |
| ECHDC3 | 693484II [symbol] | 0.605 | 1.358 | 1.153 | 1.837 | 0.264 | −0.286 | 1.767 | 1.8565 | 0.381 | −0.306 | 0.698 | 1.368 | 0.347 | 0.333 | 0 | 0.67 |
| EEF1A2 | 696775II [symbol] | −0.916 | −0.245 | −2.527 | −0.411 | −2.106 | −1.248 | −1.183 | −0.9185 | −1.5145 | −1.388 | 0.019 | −2.407 | −2.535 | −1.355 | −0.91 | −1.486 |
| EFHC1 | 694066II [symbol] | 0.287 | −0.057 | −0.632 | 0.536 | 1.137 | 0.436 | −0.544 | −0.1875 | 1.2505 | 0.56 | −0.117 | 0.663 | 0.06 | 0.305 | −0.036 | −0.101 |
| EFNA4 | 687429II [symbol] | 0.727 | 1.238 | 1.89 | 1.795 | 1.68 | 1.986 | 1.253 | 1.9925 | 1.154 | 1.03 | 1.246 | 1.413 | 1.44 | 1.478 | 1.03 | 2.125 |
| EF5 | 685583II [symbol] | −0.2165 | 0.9045 | 1.098 | −1.544 | 0.0215 | 0.0815 | 0.4395 | 0.4755 | 0.6755 | −1.826 | −0.5255 | −0.0215 | −0.7885 | −0.5185 | −0.4575 | 0.4055 |
| EIF2C2 | 696794II [symbol] | 0.114 | −0.193 | 0.694 | −0.638 | −0.576 | 1.292 | 0.789 | 0.214 | −0.3285 | −0.519 | −0.166 | −0.173 | −0.5615 | −0.697 | −0.611 | 0.218 |
| EIF2C4 | 689728II [symbol] | −0.1845 | −1.008 | 1.079 | −0.5245 | −1.145 | 0.1155 | 0.4955 | 0.087 | −0.5555 | −0.4845 | 0.1155 | 0.1555 | −0.2855 | −0.3245 | −0.2515 | 0.5155 |
| EIF4EBP1 | 683217II [symbol] | 0.864 | −0.298 | −0.703 | −0.577 | 0.656 | −0.537 | 1.86 | 1.0445 | 0.12 | −1.096 | 0.052 | 0.769 | 0.0165 | −0.883 | 0.303 | 0.004 |
| EIF4G1 | 692045II [symbol] | −0.1355 | 0.0615 | 0.8865 | 0.4625 | −0.3255 | 0.4505 | −0.5355 | 0.2105 | −0.0965 | 0.3355 | 0.3205 | −0.7775 | −0.2655 | 0.4595 | 0.0085 | 0.0065 |
| EIF4G3 | 683768II [symbol] | −0.854 | 0.122 | 0.294 | 0.005 | 0.423 | −0.005 | −0.135 | −0.294 | −0.5375 | 0.496 | −0.12 | 0.353 | −0.6685 | 0.352 | 0.268 | 0.144 |
| ELAC1 | 695486II [symbol] | −0.378 | −1.029 | −0.967 | −0.259 | −0.069 | −1.397 | −0.762 | −0.0065 | −0.434 | −0.79 | −1.182 | 0.195 | −0.4635 | −0.05 | −0.635 | −0.046 |
| ELL2 | 684066II [symbol] | −0.6585 | −0.0575 | −0.0785 | −0.2555 | 1.886 | 0.3885 | −0.1765 | 0.1865 | −0.1555 | −0.1775 | 0.6345 | 0.3155 | 1.002 | 1.406 | 0.4545 | 0.9525 |
| ELL3 | 695507II [symbol] | −0.8568 | 0.0813 | 0.0103 | 0.2112 | −0.1498 | −0.7278 | −1.048 | 0.2832 | −0.5408 | −0.3078 | −0.1208 | −0.3958 | −0.0103 | 0.0873 | −0.8388 | 0.2282 |
| ELOVL5 | 694593II [symbol] | −1.403 | 0.039 | −0.287 | −0.018 | 1.253 | −0.459 | −1.854 | −1.457 | −0.514 | 2.436 | 1.208 | 0.512 | −0.065 | 1.51 | −0.223 | 0.472 |
| EN1 | 689084II [symbol] | −0.2295 | −0.4825 | −0.4755 | 0.5695 | 0.3545 | −0.0705 | 0.6455 | 0.885 | −0.433 | −0.2805 | 0.0095 | −0.1535 | −0.167 | 0.4075 | 0.2175 | 0.0385 |
| ENAH | 682255II [symbol] | −0.019 | −0.319 | 0.156 | −1.163 | 0.598 | −1.332 | 0.439 | 0.312 | −0.8545 | −0.232 | 0.381 | 1.105 | 0.4255 | −0.889 | −0.329 | 0.321 |
| EPHX2 | 683128II [symbol] | −0.019 | −0.158 | −0.837 | 0.019 | 1.321 | 1.326 | −1.272 | −1.525 | 0.1675 | 1.037 | 0.573 | 1.567 | 0.603 | 1.332 | −0.099 | −0.21 |
| EPLIN | 687610II [symbol] | 1.098 | 1.017 | −0.6645 | −0.3655 | −0.2025 | 0.3695 | −0.3155 | −0.0585 | −0.639 | 0.1795 | 1.221 | 0.3235 | 1.345 | 0.0585 | 0.6455 | 1.014 |
| EPN3 | 691782II [symbol] | 0.192 | −0.173 | 0.492 | −0.959 | 0.029 | 0.191 | −0.315 | −0.9085 | −0.512 | 2.326 | 0.529 | 0.828 | 0.741 | −0.321 | 0.212 | 0.541 |
| EPOR | 684991II [symbol] | 1.272 | 0.8425 | −0.7366 | −0.6555 | 1.212 | −0.0715 | −0.4675 | 0.6555 | −0.0315 | −0.5735 | 0.3235 | 1.469 | 1.569 | −0.0525 | 1.506 | 0.1965 |
| ERBB2 | 688427II [symbol] | −1.068 | −0.167 | −0.548 | −0.14 | −0.423 | 4.56 | −1.249 | 4.415 | 1.13 | 1.358 | −0.86 | 1.175 | 0.254 | −0.515 | 1.077 | 3.685 |
| ERP70 | 696269II [symbol] | −2.074 | −0.692 | 0.062 | −0.721 | −0.696 | −0.161 | −0.774 | −1.2475 | −1.6965 | −0.599 | 0.01 | −0.532 | −0.3795 | −0.894 | −0.532 | −0.843 |
| ESR1 | 765041II [symbol] | 0.765 | 0.095 | 1.51 | −0.932 | 1.64 | 0.696 | 1.845 | −0.033 | 0.8815 | −0.385 | 1.218 | 1.838 | 1.231 | 0.993 | 1.372 | 0.766 |
| ETFA | 693087II [symbol] | −2.615 | −1.752 | −0.918 | −1.798 | −3.124 | −1 | −1.829 | −0.9845 | −2.0885 | −1.662 | −1.306 | −1.297 | −2.257 | −1.874 | −2.067 | −0.713 |
| ETV6 | 697286II [symbol] | 0.568 | 1.459 | 1.533 | 1.502 | 0.835 | 1.614 | 2.864 | 1.269 | 1.134 | 0.841 | 1.349 | 1.039 | 1.221 | 0.697 | 1.505 | 1.348 |
| EVER1 | 688220II [symbol] | −0.0495 | −0.9925 | 0.0495 | −0.6445 | −0.1715 | 1.922 | 0.8785 | 0.7175 | −0.281 | 0.4585 | 0.2265 | −1.39 | −0.549 | −0.0815 | 0.9245 | 0.2705 |
| EXO1 | 689742II [symbol] | 0.017 | 0.693 | 0.164 | 1.149 | 0.646 | 0.005 | −0.132 | −0.201 | 0.1165 | 0.253 | 1.206 | −0.409 | 0.0815 | −0.651 | 0.186 | −0.093 |
| EXOSC9 | 691275II [symbol] | −0.6 | −0.112 | 0.958 | −0.442 | −0.511 | −0.044 | 0.535 | 0.054 | 0.4585 | 0.044 | 0.274 | −0.204 | −0.5765 | 0.096 | −0.391 | 0.22 |
| EXT2 | 693031II [symbol] | 0.186 | 0.402 | 0.345 | 0.05 | −1.078 | 0.265 | 0.934 | −0.09 | −0.1905 | −0.019 | 0.45 | 0.042 | −0.079 | 0.383 | 0.434 | 0.071 |
| EZH2 | 689533II [symbol] | −1.041 | −0.866 | −0.151 | −0.59 | −0.798 | −0.133 | −0.324 | −0.2305 | −0.194 | −0.046 | 0.084 | −0.483 | −0.5335 | −0.33 | −0.067 | −0.164 |
| F11R | 684061II [symbol] | 0.118 | 0.689 | 0.174 | 0.829 | 1.06 | 0.972 | −0.106 | 0.9955 | 1.992 | 1.318 | 1.173 | 0.412 | 0.408 | 0.799 | 0.58 | 0.713 |
| F9 | 682839II [symbol] | −0.331 | 0.577 | 0.199 | 1.521 | 0.49 | −0.587 | −0.439 | 0.036 | 0.5675 | 0.966 | −0.025 | 0.562 | 0.75 | 1.433 | −0.339 | 0.257 |
| FA2H | 688063II [symbol] | −0.608 | −0.778 | 3.279 | 0.392 | −0.831 | 2.375 | −0.336 | 1.103 | 0.508 | 2.308 | 0.407 | −0.502 | 0.121 | −1.409 | 0.589 | 0.765 |
| FABP7 | 694559II [symbol] | −0.544 | −2.649 | −1.524 | −1.389 | −2.645 | −1.556 | 4.58 | −3.032 | −1.944 | −2.184 | −2.207 | −1.472 | −2.021 | −2.698 | −1.606 | −1.407 |
| FADD | 686191II [symbol] | 1.133 | 0.739 | −0.073 | −0.056 | −0.07 | −0.083 | 0.23 | 0.5035 | 0.046 | 0.061 | 0.546 | 0.187 | −0.2375 | −0.172 | −0.197 | 0.16 |
| FAHD1 | 688442II [symbol] | −0.727 | −0.254 | −0.369 | 0.577 | 0.299 | −0.133 | −0.192 | −0.172 | −0.055 | 0.003 | −0.222 | 0.941 | −0.337 | −0.084 | −0.486 | 0.702 |
| FAHD2A | 687850II [symbol] | −0.3105 | −0.0565 | 0.2345 | −0.1315 | −0.2955 | 0.5165 | −0.5185 | 0.1825 | −0.4965 | −0.3275 | −0.0015 | −0.3605 | 0.4035 | 0.2435 | 0.2885 | −0.2715 |
| FAM36A | 683002II [symbol] | −0.709 | −0.003 | 0.027 | 0.3 | 1.503 | −0.686 | −0.189 | 0.2415 | 0.4075 | −0.369 | −0.45 | 0.003 | −0.717 | 0.036 | −0.408 | −0.469 |
| FAM3A | 695471II [symbol] | 0.415 | 0.131 | 0.19 | −0.427 | −0.076 | 0.138 | −0.068 | −0.0715 | −0.07 | 1.821 | −0.014 | 0.208 | 0.5115 | −0.022 | 0.479 | 0.105 |
| FAM3B | 685659II [symbol] | 0.8135 | 0.0415 | 0.2715 | −0.0595 | −0.9605 | −0.0415 | 0.4426 | −0.936 | 0.282 | 4.054 | 3.852 | −0.8665 | 0.342 | −0.7385 | 0.6665 | 2.181 |
| FAM3C | 681623II [symbol] | −0.827 | 0.024 | −0.49 | −0.793 | −0.848 | −0.663 | −0.112 | −0.4615 | −1.101 | −0.024 | 0.878 | −0.227 | −0.448 | 0.088 | −0.378 | −0.228 |
| FAM46C | 685310II [symbol] | −0.1475 | 0.9685 | −0.1985 | −0.5185 | 1.452 | 1.176 | −0.5995 | 0.042 | −0.5695 | 1.175 | 0.3055 | −0.0425 | 0.5765 | 0.7195 | 0.5876 | 0.3605 |
| FAM49B | 692836II [symbol] | −0.571 | −0.285 | −0.062 | −0.808 | −0.184 | −0.913 | −0.861 | −1.3525 | −1.802 | −0.576 | −0.459 | −1.126 | −0.0115 | −1.158 | −1.35 | 0.501 |
| FAM54A | 690904II [symbol] | 0.193 | 0.678 | 0.064 | −0.205 | −0.241 | −0.482 | 0.673 | 0.625 | 0.1445 | 0.134 | 0.49 | 0.274 | 0.281 | 0.067 | −0.126 | 0.428 |
| FANCA | 685878II [symbol] | −0.395 | 0.429 | 0.653 | −0.095 | −1.373 | 0.465 | 1.755 | 1.313 | −0.083 | 0.045 | 0.645 | −0.785 | −0.3365 | −0.895 | −0.371 | −0.045 |
| FANCE | 692746II [symbol] | −1.282 | −2.091 | −0.538 | −1.635 | −1.869 | −0.917 | −0.316 | −1.1335 | −1.4485 | −1.281 | −1.441 | −2.467 | −1.6425 | −2.494 | −1.545 | −1.207 |
| FBL | 682815II [symbol] | 0.3855 | 0.2255 | −0.3435 | −0.4065 | −0.2315 | 0.3025 | 0.5025 | 0.476 | −0.3715 | −0.6035 | −0.2005 | 0.2935 | 0.2965 | −0.3405 | −0.0225 | −0.0985 |
| FBP1 | 687231II [symbol] | 0.6633 | 0.7763 | −0.0668 | −0.5117 | −0.5787 | 0.7233 | 1.537 | 0.0668 | −1.087 | −0.7587 | 0.4873 | −1.221 | 0.3988 | −0.1527 | −0.4407 | 0.4203 |
| FBS1 | 683642II [symbol] | −0.551 | −0.02 | −0.5 | −0.112 | 0.236 | −0.201 | 0.178 | −0.12 | 0.2055 | 0.19 | 0.105 | 0.458 | −0.101 | −0.271 | 0.769 | 0.096 |
| FBXL20 | 696291II [symbol] | −0.39 | −0.174 | −0.122 | −0.156 | −0.068 | −0.057 | −0.149 | 1.9205 | −0.0435 | −0.32 | −0.568 | 0.385 | 0.3695 | 0.031 | −0.176 | 1.743 |
| FBXO25 | 682373II [symbol] | −0.385 | −0.006 | −1.113 | 0.7 | 0.006 | 0.098 | 0.064 | −0.192 | 0.3875 | 0.409 | −0.132 | 0.211 | 0.1015 | 0.167 | −0.526 | −0.636 |
| FDXR | 696354II [symbol] | 0.054 | −0.503 | −0.286 | 0.12 | −0.154 | −0.684 | −1.024 | −0.4865 | 0.3045 | 1.28 | −1.234 | 0.075 | 0.2155 | −0.174 | −0.034 | −1.172 |
| FEM1B | 687319II [symbol] | −0.447 | 0.387 | −0.087 | −0.662 | −0.546 | −0.351 | −0.308 | 0.5115 | −0.027 | 0.29 | 0.472 | 0.184 | 0.055 | 0.038 | −0.134 | 0.445 |
| FGF12 | 686589II [symbol] | −0.0435 | 1.427 | −0.2525 | −0.0325 | 0.1915 | −0.1995 | 1.567 | 2.402 | 1.755 | 0.8435 | 0.6385 | −0.5605 | 2.115 | 2.538 | 0.1115 | −0.2255 |
| FGF13 | 696768II [symbol] | 1.061 | −0.365 | −0.184 | −0.106 | −0.737 | 0.476 | 0.916 | −0.327 | −1.333 | −0.404 | 0.536 | −0.925 | 0.36 | −0.756 | −0.121 | 0.421 |
| FHOD1 | 693587II [symbol] | −0.163 | −0.391 | 1.09 | −0.116 | −0.245 | 0.103 | −0.354 | 0.3605 | −0.098 | −0.158 | −0.123 | −0.461 | −0.896 | −0.115 | 0.076 | −0.332 |
| FKBP11 | 684453II [symbol] | −0.3445 | 0.0285 | 0.4705 | −0.3805 | −0.1545 | 0.0645 | 0.6015 | 0.319 | 0.1525 | 0.4605 | 0.1885 | 0.1035 | −0.2415 | −0.0285 | 0.0475 | 1.29 |
| FLJ10094 | 684307II [symbol] | −0.7885 | 0.0385 | −0.3835 | 3.429 | −0.2555 | −0.3705 | −0.6925 | 0.481 | 0.739 | −0.4605 | −0.2555 | 0.9805 | 0.1185 | 2.063 | −0.0365 | −0.0335 |
| FLJ10116 | 682368II [symbol] | −0.6175 | 0.6335 | −0.9175 | −0.9845 | 0.5185 | −1.14 | −1.216 | −1.633 | −0.868 | 0.1755 | 0.2495 | 0.9715 | −0.091 | 0.8935 | 0.7715 | −0.0355 |
| FLJ10159 | 685392II [symbol] | 1.958 | 0.124 | −0.374 | −0.334 | −0.238 | −0.027 | 1.931 | 1.414 | 0.4445 | 0.87 | 0.324 | 0.253 | 0.027 | 0.464 | 0.706 | 0.859 |
| FLJ10204 | 688075II [symbol] | −0.3835 | 0.2315 | −0.5355 | −0.0765 | −0.3865 | 1.848 | −0.2175 | 0.5805 | −0.484 | −0.0685 | −0.0405 | 0.7015 | −0.599 | −0.1875 | −0.5305 | 0.5435 |
| FLJ10276 | 694528II [symbol] | 0.951 | 0.857 | 0.461 | 0.969 | 0.803 | 1.447 | 0.679 | 1.3025 | 0.965 | 1.245 | 0.71 | 0.857 | 0.41 | 0.74 | 0.593 | 0.465 |
| FLJ10292 | 686906II [symbol] | 0.514 | −0.075 | −0.103 | −0.283 | −0.431 | −0.845 | 1.139 | −0.4225 | −0.3305 | 0.187 | 0.96 | 0.892 | −0.6695 | −0.58 | −0.882 | −0.129 |
| FLJ10324 | 693717II [symbol] | 0.976 | 0.782 | 0.476 | −0.605 | 0.928 | −1.139 | −0.219 | −0.087 | −0.2305 | 0.352 | 0.049 | −0.546 | 1.1095 | 1.836 | −0.194 | −0.033 |
| FLJ10700 | 687078II [symbol] | −0.1715 | 0.3135 | −0.0515 | −0.4075 | 0.3835 | 0.8835 | 0.4765 | −0.7116 | 0.0525 | 0.6575 | −0.2815 | 0.7635 | −0.582 | 0.1025 | 0.0515 | −0.4595 |
| FLJ10706 | 693300II [symbol] | −1.931 | −1.369 | −1.237 | −2.341 | −1.335 | −1.233 | −0.58 | −0.7365 | −0.8285 | −1.837 | −1.232 | −0.566 | −0.44 | −1.93 | −1.442 | −0.608 |
| FLJ10871 | 694856II [symbol] | 0.354 | 0.471 | −0.452 | 0.214 | 0.252 | 0.6 | −0.025 | 0.1675 | 0.077 | 0.196 | 0.369 | 0.888 | 1.1025 | 0.118 | −0.064 | −0.217 |
| FLJ10901 | 684051II [symbol] | −0.2655 | −1.625 | 0.8645 | −1.589 | −2.142 | 0.6595 | 0.4075 | −0.0805 | −1.773 | 0.0805 | −0.7665 | −1.457 | −1.579 | −2.008 | 0.6495 | 1.702 |
| FLJ10916 | 695840II [symbol] | −0.383 | −0.191 | 0.096 | 0.739 | −0.225 | −0.227 | 0.369 | 0.9545 | −0.104 | −0.194 | 0.073 | −0.45 | −0.497 | 0.151 | −0.616 | 0.415 |
| FLJ10960 | 684797II [symbol] | −0.8005 | −0.6535 | −0.8105 | 0.8845 | 1.147 | −0.0195 | −1.016 | −1.382 | 1.169 | 1.068 | 0.7385 | 2.065 | 0.5505 | −0.1945 | 0.0195 | 0.8615 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| FLJ11017 | 682528II [symbol] | 0.829 | 0.175 | 0.151 | 0.688 | −0.779 | −0.595 | 0.595 | 0.625 | 0.1255 | 1.119 | 1.161 | 0.394 | −0.0865 | 0.296 | 0.613 | 0.731 |
| FLJ11088 | 689859II [symbol] | 0.063 | −0.177 | −0.986 | −0.165 | 0.68 | −0.696 | −0.719 | −0.566 | −0.045 | 0.949 | 0.06 | 0.011 | −0.252 | 1.737 | −0.285 | 0.329 |
| FLJ11151 | 695680II [symbol] | −0.597 | −0.72 | 0.033 | −1.434 | −0.757 | −1.37 | 0.208 | −0.8315 | −1.7755 | −1.205 | −0.264 | 0.184 | −1.2015 | −0.813 | −0.834 | 0.455 |
| FLJ11287 | 688538II [symbol] | −0.0725 | 0.0845 | −0.2285 | −0.1615 | 0.1485 | −0.2625 | −0.1165 | −0.1185 | 0.119 | −0.0005 | −0.9355 | −0.2505 | 0.066 | 0.0165 | 0.1935 | −0.0015 |
| FLJ11280 | 697294II [symbol] | 0.528 | 1.432 | 0.692 | 1.149 | 2.016 | 0.384 | 0.271 | −0.1055 | 1.383 | 1.64 | 1.022 | 2.042 | 0.9075 | 1.368 | 1.964 | 0.331 |
| FLJ11506 | 691255II [symbol] | −0.0005 | 0.7945 | 0.1275 | 0.2975 | −0.0045 | −0.0715 | −0.0865 | 0.3575 | 0.136 | 0.4275 | 0.1185 | −0.1695 | −0.1085 | 0.1365 | 0.0445 | 0.1925 |
| FLJ12270 | 690555II [symbol] | 0.4633 | −0.0728 | 0.1203 | 0.0543 | −0.4477 | 0.0123 | 0.0323 | −0.0123 | −1.171 | 0.2333 | −0.1317 | −0.0958 | −0.5682 | −0.1987 | 0.2133 | −0.1247 |
| FLJ12644 | 693818II [symbol] | 0.797 | −0.076 | −0.334 | −0.312 | −0.221 | −0.457 | 0.332 | 0.188 | −0.118 | −0.39 | 1.043 | −0.074 | −0.134 | −0.166 | 0.043 | 0.447 |
| FLJ12950 | 690008II [symbol] | −0.0295 | 3.068 | −1.406 | −1.335 | 0.0295 | −0.9905 | 0.1795 | −0.6135 | 0.1735 | 0.4365 | 2.209 | 2.803 | 2.281 | 2.365 | 1.195 | −1.748 |
| FLJ12684 | 685216II [symbol] | −0.242 | −0.378 | −0.08 | −0.438 | −0.044 | 0.097 | 0.224 | −0.0705 | 0.19 | 0.891 | −0.257 | −0.182 | −0.1775 | −0.112 | −0.146 | 0.079 |
| FLJ12735 | 697973II [symbol] | −0.3785 | 0.3445 | −0.5385 | −0.0965 | −0.2295 | −0.8035 | −0.0685 | 0.115 | −0.8315 | 0.3445 | 0.2275 | 0.3325 | 0.0415 | −0.3295 | −0.2145 | 0.2905 |
| FLJ12750 | 685380II [symbol] | −0.0145 | −0.2895 | 1.147 | −0.0645 | −0.0805 | 1.05 | 1.447 | 1.038 | −0.2835 | −0.3615 | 0.3375 | 0.0725 | −0.069 | −0.5865 | 0.1535 | 1.089 |
| FLJ12895 | 683723II [symbol] | 0.3775 | 0.1055 | −0.4645 | −0.0135 | 0.0135 | 0.3785 | 0.2705 | −0.432 | −0.929 | 0.9235 | 0.9135 | 0.4305 | −0.5575 | −1.273 | 0.4625 | −0.2845 |
| FLJ13710 | 693274II [symbol] | 0.727 | 1.977 | −1.427 | 2.486 | 2.119 | −0.595 | 0.023 | −0.605 | 1.6505 | 3.289 | 2.095 | 1.944 | 1.4515 | 2.361 | 1.549 | 0.39 |
| FLJ13855 | 681758II [symbol] | 0.02 | 0.071 | −0.116 | 0.499 | −0.125 | −0.338 | −0.072 | 0.524 | −0.8795 | 0.117 | −0.416 | −0.168 | 0.16 | −0.203 | 0.121 | 0.025 |
| FLJ13912 | 696739II [symbol] | −1.95 | −2.53 | 0.16 | −2.119 | −2.867 | −2.307 | −0.955 | −2.2415 | −2.0385 | −0.67 | −0.935 | −1.595 | −1.978 | −2.052 | −1.459 | −1.758 |
| FLJ14124 | 691127II [symbol] | −0.687 | 0.166 | 0.011 | −0.204 | 0.544 | −0.107 | −0.56 | 0.047 | 0.026 | −0.672 | −0.139 | 0.334 | 0.033 | −0.465 | −0.208 | −0.021 |
| FLJ14627 | 681398II [symbol] | −0.913 | 0.428 | 0.377 | −0.131 | −0.295 | −0.129 | −0.019 | 0.1775 | −0.0095 | −0.362 | 0.263 | −0.005 | −0.448 | 0.16 | 0.174 | 0.005 |
| FLJ14668 | 696627II [symbol] | −0.954 | −0.842 | −0.355 | −0.68 | −0.749 | −0.765 | −0.018 | −0.2905 | 0.4855 | −0.559 | −0.407 | −1.221 | 0.8855 | −0.773 | −0.721 | −0.656 |
| FLJ14904 | 687755II [symbol] | −0.0123 | 0.2527 | −0.0123 | 0.2697 | 0.0638 | 0.0568 | −0.2023 | 2.278 | 0.0983 | −0.3983 | −0.2753 | −0.2173 | 0.0123 | −0.2513 | −0.2363 | −0.1833 |
| FLJ20035 | 696171II [symbol] | 0.494 | 1.708 | 2.234 | 1.359 | 0.62 | 0.817 | 1.35 | 1.0465 | −0.53 | 2.784 | 1.859 | 1.607 | 0.5135 | 0.918 | 1.202 | 1.961 |
| FLJ20152 | 694420II [symbol] | 0.994 | 1.472 | −1.157 | 1.697 | 2.011 | −0.878 | −1.012 | −0.7995 | 0.5035 | 3.104 | 2.02 | −0.896 | 1.7795 | 1.245 | 1.57 | 0.065 |
| FLJ20171 | 695239II [symbol] | 0.026 | 0.452 | −0.799 | 0.86 | 0.126 | −0.699 | −0.558 | −0.427 | 0.2185 | 0.83 | 0.228 | 0.219 | 1.166 | 1.424 | 0.019 | −0.133 |
| FLJ20244 | 684905II [symbol] | 0.186 | 0.107 | 0.156 | −0.336 | −0.115 | 0.293 | 0.804 | 0.406 | −0.198 | −0.125 | −0.613 | 0.052 | 0.2815 | −0.313 | −0.061 | −0.191 |
| FLJ20273 | 683092II [symbol] | −1.373 | 0.287 | −0.823 | −1.086 | −0.274 | 0.12 | −0.667 | −0.179 | −1.501 | 0.041 | 0.784 | 1.282 | 0.6575 | 0.138 | 0.168 | 1.198 |
| FLJ20315 | 689446II [symbol] | −0.155 | 1.484 | 0.625 | 1.378 | −0.919 | −0.934 | 1.431 | −0.255 | −0.258 | 2.013 | 0.948 | 0.123 | 0.991 | −1.262 | 0.842 | 2.008 |
| FLJ20323 | 697352II [symbol] | 0.161 | 0.257 | −0.243 | −0.436 | 0.177 | −0.047 | 0.037 | −0.423 | −0.6115 | 0.38 | 1.268 | 0.328 | 1.075 | −0.276 | 0.726 | −0.248 |
| FLJ20516 | 688292II [symbol] | −1.338 | −0.3625 | 0.8455 | −0.4195 | 0.1575 | 0.0645 | 0.8645 | 0.0085 | 0.354 | −0.6605 | 0.2345 | 0.4675 | −0.407 | 0.3695 | −0.3945 | 0.0765 |
| FLJ20530 | 681463II [symbol] | −0.134 | −0.245 | 0.107 | −0.321 | −0.003 | 1.764 | −0.534 | 0.0135 | −0.916 | −0.268 | −0.203 | 0.502 | −0.4835 | −0.452 | −0.712 | 0.68 |
| FLJ20696 | 684792II [symbol] | 0.799 | −0.841 | −0.735 | 0.317 | 0.373 | −0.034 | 0.133 | −1.11 | −0.4775 | −0.036 | −0.058 | 0.604 | 0.283 | −0.763 | 0.034 | 0.463 |
| FLJ20718 | 692255II [symbol] | −2.138 | −1.08 | 0.234 | −0.445 | −1.723 | −0.849 | 0.655 | −0.1405 | −0.026 | 1.006 | 1.81 | 0.223 | 1.326 | −0.497 | −0.735 | 0.655 |
| FLJ20772 | 688898II [symbol] | −0.108 | 0.079 | 0.865 | −0.074 | −0.13 | 2.371 | −0.376 | 0.0515 | −0.399 | −0.746 | −0.192 | 0.062 | −0.1865 | 0.925 | −0.053 | 0.458 |
| FLJ20989 | 695210II [symbol] | 0.069 | 0.165 | −0.095 | 0.029 | 0.074 | 0.566 | −0.17 | 0.161 | −0.093 | 0.068 | 0.04 | −0.098 | −0.074 | 0.046 | −0.004 | 0.224 |
| FLJ21019 | 689334II [symbol] | 0.1095 | 0.2185 | 0.5935 | −0.1175 | 0.5285 | −0.9315 | −0.0155 | 0.519 | 0.125 | 0.4215 | 0.0155 | 0.3185 | 0.3265 | −0.0755 | 1.03 | −0.5475 |
| FLJ21052 | 695900II [symbol] | 0.242 | 0.929 | −0.014 | 0.239 | 1.511 | 0.456 | −0.334 | 0.5885 | 1.042 | 1.343 | 0.478 | 1.722 | 1.843 | 0.536 | 0.861 | 0.509 |
| FLJ21159 | 688476II [symbol] | −0.5825 | −0.9215 | −0.0595 | 0.6815 | −1.122 | −0.7975 | −0.2705 | −0.5175 | 0.672 | 0.7665 | −0.2475 | 0.6715 | −1.777 | 0.9345 | 0.5815 | 0.7145 |
| FLJ21616 | 696280II [symbol] | 0.388 | −0.085 | −1.796 | −1.566 | 1.335 | 0.429 | 0.201 | −1.367 | −0.295 | −0.241 | 0.307 | 1.306 | 1.0425 | 0.351 | 0.32 | −0.446 |
| FLJ21827 | 683572II [symbol] | 0.1365 | −0.2205 | −0.1545 | 1.102 | 0.6985 | −0.0585 | −0.0115 | −0.654 | 0.5355 | −0.2085 | 0.2505 | 0.7945 | 0.2075 | 0.9245 | 0.1255 | −0.1525 |
| FLJ21963 | 688106II [symbol] | 0.704 | −0.004 | 0.974 | −0.136 | −0.641 | 0.484 | 0.844 | −0.815 | −0.28 | −0.056 | −0.206 | 0.004 | 0.7065 | −0.486 | 0.105 | 0.354 |
| FLJ22104 | 690673II [symbol] | 0.051 | −0.038 | −0.028 | −0.16 | 0.028 | −0.35 | −0.569 | −1.214 | −0.4485 | 0.302 | 0.516 | 1.061 | 0.068 | −0.302 | −0.189 | 0.634 |
| FLJ22222 | 694558II [symbol] | −0.678 | 0.097 | 0.08 | 0.434 | 0.004 | 0.72 | 0.641 | 0.4005 | 0.097 | 1.075 | −0.229 | −0.107 | 0.797 | 1.473 | 0.175 | −0.237 |
| FLJ22573 | 683583II [symbol] | −0.0108 | 0.3952 | 1.075 | 0.4952 | 0.6412 | 0.6802 | 0.5612 | 0.0773 | 0.2712 | −0.1558 | −0.5928 | −0.1258 | 0.0108 | 0.2512 | 0.2722 | 1.092 |
| FLJ22794 | 692448II [symbol] | −0.003 | 0.452 | 0.404 | 0.643 | 0.934 | 0.939 | −0.205 | 0.5935 | 1.76 | 0.728 | 0.127 | 0.246 | 0.3045 | −0.717 | 0.44 | 0.89 |
| FLJ23186 | 692984II [symbol] | 0.167 | 0.802 | −0.133 | 0.936 | 0.724 | −0.157 | −0.129 | −0.4435 | 0.6575 | 2.603 | 1.481 | −0.335 | 0.9715 | −0.189 | 0.575 | 0.538 |
| FLJ23441 | 696416II [symbol] | 0.246 | 0.784 | 0.233 | 0.543 | 1.515 | −0.045 | 0.158 | 0.336 | 0.576 | 0.549 | 0.26 | 0.635 | 0.8525 | 0.809 | 0.693 | 0.405 |
| FLJ25471 | 695557II [symbol] | −2.007 | −0.587 | 0.026 | −1.011 | −0.811 | −0.223 | 0.297 | 0.0475 | −0.8925 | −1.218 | 0.245 | −0.475 | 0.014 | −0.892 | −1.026 | −0.587 |
| FLJ31204 | 683963II [symbol] | 0.072 | −0.273 | −0.684 | 1.074 | 0.888 | −0.863 | −1.126 | −0.757 | −0.117 | 1.159 | −0.125 | 0.411 | −0.123 | 0.875 | −0.924 | 0.111 |
| FLJ31795 | 688302II [symbol] | −0.3375 | −0.8225 | 0.6115 | −0.3665 | −0.5375 | −0.3865 | 0.4195 | −0.865 | −0.9255 | 0.3565 | 0.0815 | 0.7875 | −0.1845 | 0.0065 | 0.0875 | 0.5325 |
| FLJ32942 | 690691II [symbol] | 1.168 | −0.191 | −0.106 | 0.208 | −0.192 | 0.458 | 0.119 | −0.363 | 0.3925 | −0.357 | −0.381 | −0.154 | 0.028 | −0.156 | 0.338 | −0.17 |
| FLJ37970 | 697729II [symbol] | 0.294 | −0.315 | 0.336 | −1.021 | −1.264 | −0.415 | 0.786 | −0.1165 | −0.5005 | −1.016 | −0.446 | −1.029 | −1.623 | −0.023 | −1.028 | 0.028 |
| FLJ39370 | 694709II [symbol] | 1.216 | 1.098 | 1.079 | 0.162 | 1.781 | 0.091 | 1.172 | −1.7935 | 2.2845 | 1.478 | 0.955 | 1.116 | 2.0705 | 0.816 | 0.594 | 0.427 |
| FLJ90586 | 685670II [symbol] | 0.782 | −1.988 | −2.148 | −0.982 | −1.325 | −1.041 | −1.944 | 0.043 | −1.335 | 0.234 | 1.206 | −0.043 | −0.7335 | −0.623 | 0.578 | 0.936 |
| FN3KRP | 695326II [symbol] | −0.323 | −0.521 | −0.581 | −0.403 | −0.61 | −0.393 | 0.968 | −0.7095 | −0.6625 | 0.482 | −0.029 | 0.172 | 0.156 | −0.409 | 0.251 | −0.302 |
| FOLR1 | 697855II [symbol] | −0.214 | −1.592 | −1.927 | −1.245 | −1.97 | −1.521 | 0.251 | −1.7245 | −1.4215 | −1.983 | −2.615 | −2.278 | −1.968 | −2.45 | −1.134 | 1.553 |
| FOXA1 | 692889II [symbol] | 0.51 | 1.605 | 1.547 | 2.72 | 2.608 | 2.37 | −3.19 | 0.6095 | 1.864 | 3.55 | 2.65 | 2.65 | 2.4265 | 3.38 | 2.148 | 1.8 |
| FOXC1 | 772435II [symbol] | 0.6762 | −0.41 | −0.71 | 0.008 | −0.78 | −0.076 | 3.622 | −1.12 | −0.42 | −1.078 | −1.304 | −1.761 | −1.22 | 0.074 | 0.3 | −0.276 |
| FOXP1 | 692043II [symbol] | 0.512 | 0.77 | −0.148 | −0.637 | 0.891 | 1.119 | −0.447 | 1.285 | 1.276 | 0.001 | −0.364 | −0.001 | 1.043 | 0.302 | 0.856 | 0.203 |
| FREQ | 683314II [symbol] | −1.042 | −0.0223 | 0.6058 | −0.2912 | −0.6682 | 0.4098 | 0.4598 | 0.0223 | −0.5602 | 0.4438 | 0.5858 | −0.5352 | −0.8367 | −0.3322 | −0.6632 | −0.0233 |
| FTO | 693312II [symbol] | 0.399 | 0.16 | 0.884 | 1.977 | −0.115 | −0.174 | −0.069 | 0.317 | −0.4205 | 0.622 | 0.447 | −0.071 | 0.0355 | 1.94 | 0.844 | 0.491 |
| FUBP1 | 690423II [symbol] | −0.647 | −0.094 | 0.368 | 0.643 | 0.32 | −0.232 | 0.42 | −0.9265 | −0.7235 | 0.637 | 0.845 | 0.486 | −0.5035 | 0.176 | −0.301 | 0.424 |
| FUCA1 | 691254II [symbol] | −0.0085 | 0.3025 | −0.0215 | 0.0645 | 0.9035 | −0.3615 | −1.34 | −0.1935 | 0.0085 | 0.8175 | 0.2305 | 0.6295 | 0.967 | 0.7445 | −0.3725 | 0.2335 |
| FUT8 | 695197II [symbol] | −0.381 | 0.051 | −0.437 | 0.193 | 0.968 | −0.052 | −0.576 | −0.806 | 0.624 | 0.63 | 0.537 | 0.621 | 0.0735 | 0.368 | 0.796 | −0.398 |
| FXYD8 | 687048II [symbol] | 0.5065 | 0.0615 | −0.5595 | −0.0225 | −0.8395 | 0.1295 | 1.287 | 1.2 | 0.7475 | −0.2065 | 0.1815 | −0.1155 | −0.4125 | 0.1855 | 0.3355 | 0.3945 |
| FYCO1 | 691750II [symbol] | 0.276 | 0.283 | −0.158 | −0.314 | 0.995 | 0.211 | −0.448 | 0.128 | −0.228 | 0.326 | −0.316 | 0.919 | 0.2195 | −0.062 | −0.026 | 1.194 |
| FZD10 | 686248II [symbol] | −0.078 | −0.173 | −0.526 | 0.058 | −0.012 | 0.584 | −0.228 | 0.2335 | 1.137 | −0.778 | 0.457 | −0.184 | −0.418 | 2.47 | 0.068 | −0.324 |
| FZD7 | 691443II [symbol] | 1.206 | 0.032 | −1.205 | −1.2 | −0.004 | −1.005 | 0.188 | −0.685 | −0.599 | 0.163 | 0.683 | 0.687 | −0.2195 | 0.236 | 0.309 | 0.442 |
| FZD9 | 691926II [symbol] | 0.277 | −0.432 | −0.333 | −0.565 | −0.388 | −0.042 | 0.624 | −0.543 | −0.595 | 0.145 | 0.24 | 0.715 | −0.676 | −0.595 | −0.299 | −0.099 |
| G1P2 | 694822II [symbol] | 0.462 | 1.908 | 3.723 | 3.079 | 0.714 | 2.581 | 3.215 | 4.191 | 1.8715 | 3.101 | 2.19 | 0.089 | −0.455 | 2.032 | 2.407 | 2.481 |
| G1P3 | 693731II [symbol] | 0.256 | 2.108 | 2.719 | 3.319 | 0.892 | 2.721 | 2.71 | 2.4355 | 0.809 | 3.757 | 1.885 | 0.671 | 0.4285 | 2.193 | 2.781 | 2.487 |
| GA17 | 696445II [symbol] | −1.121 | −1.421 | −1.113 | −1.262 | −1.068 | −1.063 | −1.337 | −1.8575 | −2.0785 | −1.064 | −0.193 | −0.92 | −1.4755 | −0.747 | −1.411 | −1.098 |
| GABARAF | 683657II [symbol] | 0.3915 | −0.2045 | −0.5415 | 1.111 | 0.1865 | −0.5985 | −1.283 | −0.664 | −0.9635 | 1.488 | 0.2545 | 0.1235 | −0.6995 | 1.143 | 0.2285 | −0.0105 |
| GAJ | 696722II [symbol] | −3.835 | −2.749 | −1.054 | −3.724 | −3.91 | −2.049 | −1.283 | −3.284 | −2.9965 | −2.756 | −1.689 | −3.358 | −2.8925 | −2.817 | −3.158 | −2.271 |
| GALE | 681740II [symbol] | −1.051 | 0.109 | 0.613 | −0.913 | 0.468 | −0.842 | 0.072 | 0.6475 | 0.7345 | 0.025 | −0.565 | 0.016 | −0.7885 | −0.337 | −0.752 | −0.064 |
| GALNT14 | 693376II [symbol] | 0.1755 | 0.8925 | 0.5505 | 0.2305 | −0.2105 | −0.7465 | −0.7005 | −0.6215 | 0.2285 | 0.8855 | −1.153 | 0.3045 | −0.926 | 1.336 | 1.812 | −0.0695 |
| GALNT2 | 692425II [symbol] | −0.951 | −0.272 | 0.054 | −0.782 | 0.454 | −0.106 | 0.478 | 0.3465 | 0.2595 | −0.357 | −0.556 | −0.488 | −1.0275 | −0.241 | −0.075 | −0.241 |
| GALNT7 | 684872II [symbol] | −0.517 | −0.241 | 0.796 | 0.15 | 1.235 | 0.153 | −1.544 | −0.85 | −0.033 | 0.939 | −0.083 | 1.602 | 0.319 | 1.356 | −0.539 | 1.474 |
| GAMT | 697858II [symbol] | −0.666 | −0.725 | −1.415 | −0.067 | 0.14 | 0.24 | −1.913 | −1.79 | 0.397 | −1.183 | −0.459 | 0.623 | 0.105 | 0.584 | −0.534 | −0.8 |
| GAPD | 696932II [symbol] | −1.749 | −1.459 | −0.573 | −1.078 | −2.427 | −1.772 | −1.866 | −2.412 | −3.0295 | −2.166 | −1.765 | −2.334 | −2.16 | −1.285 | −1.266 | −2.232 |
| GARS | 693904II [symbol] | −1.483 | −0.863 | 0.2 | −1.116 | −1.476 | −0.11 | 0.499 | 0.607 | −1.05 | −1.058 | −0.223 | −1.201 | −1.138 | −0.742 | −1.153 | 0.468 |
| GART | 683079II [symbol] | −0.686 | 0.007 | 1.385 | −0.396 | −0.154 | −0.109 | 0.578 | −0.297 | −1.167 | 0.351 | 0.328 | 0.405 | −0.582 | −0.139 | −0.212 | 0.196 |
| GATA3 | 696046II [symbol] | 0.254 | 1.353 | −0.803 | 2.093 | 1.715 | 0.134 | 0.49 | −1.1995 | 1.454 | 2.71 | 1.619 | 1.284 | 2.1275 | 2.502 | 2.071 | −0.529 |
| GATM | 685950II [symbol] | 0.042 | 1.005 | −0.468 | 0.213 | 1.424 | −0.955 | 0.941 | −0.7205 | −1.361 | 0.975 | 0.485 | 1.657 | −0.0975 | 1.046 | −0.374 | 1.513 |
| GBA | 691089II [symbol] | −0.6455 | 0.4885 | 0.8065 | −0.5215 | 0.7585 | 0.1105 | 0.8655 | 0.7955 | 0.284 | −0.6905 | −0.9105 | −0.0455 | −0.2785 | −0.0435 | −0.4615 | 0.1485 |
| GDAP1 | 689715II [symbol] | −0.2815 | 0.5425 | −0.0165 | 3.55 | 0.6665 | −0.1265 | −0.5925 | −1.633 | −1.237 | 0.5975 | −1.22 | 1.42 | 0.8315 | 2.25 | 0.4485 | −0.4215 |
| GDI2 | 686228II [symbol] | −0.6925 | −0.1865 | 0.7085 | −0.1715 | −0.3415 | −0.2665 | 0.8385 | 0.4785 | −0.765 | 0.0825 | 0.7535 | −0.0515 | −0.5485 | 0.3425 | −0.0535 | 0.4015 |
| GGA1 | 683921II [symbol] | −0.3755 | −0.1815 | −0.2105 | 0.1405 | 0.1675 | 0.3825 | −0.1675 | 0.67 | 0.485 | −0.7395 | −0.7015 | −0.0945 | 0.0865 | −0.1595 | −0.6145 | −0.1175 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| GKAP1 | 682770II [symbol] | −0.7235 | 0.0095 | −0.9195 | 0.7245 | 0.9165 | −0.8575 | −0.4195 | −0.1985 | 1.047 | 0.3405 | −0.1935 | −0.6085 | 0.236 | 0.4765 | −0.5885 | −0.5495 |
| GLCCI1 | 689756II [symbol] | 0.0025 | 0.7605 | −0.7195 | −1.179 | 1.239 | 0.9135 | −0.4555 | −0.0025 | 0.373 | 0.2825 | 0.2095 | 1.218 | 0.784 | −0.2785 | 0.0815 | 0.0045 |
| GLG1 | 681938II [symbol] | 0.2565 | 0.4325 | 0.5965 | 0.2155 | −0.2775 | 0.3025 | 0.1985 | 0.7875 | −0.876 | −0.2615 | 0.0095 | −0.6455 | −0.466 | 0.5435 | 0.6295 | −0.0095 |
| GLTSCR2 | 686786II [symbol] | 0.727 | 0.083 | −0.731 | 0.39 | 0.551 | 0.575 | −1.224 | −0.2765 | 0.165 | −0.168 | −0.33 | 0.423 | 0.605 | 0.582 | 0.449 | 0.105 |
| GNB4 | 697071II [symbol] | −0.394 | −0.395 | 0.547 | −1.028 | −1.066 | −1.025 | 0.429 | −0.7795 | −0.68 | −0.466 | −0.446 | −0.754 | −0.7405 | −0.461 | −0.515 | −0.57 |
| GOLPH2 | 689832II [symbol] | −0.8465 | 0.4055 | 0.2735 | −0.3875 | −0.0365 | 1.118 | −1.288 | 0.7235 | 0.1515 | −2.3 | −0.5635 | −0.7285 | 0.0185 | 0.9515 | −0.2375 | 0.4085 |
| GPATC1 | 687697II [symbol] | −0.563 | 0.211 | 0.552 | 0.028 | 0.013 | 0.589 | 0.687 | 0.802 | 0.138 | 1.223 | −0.88 | −0.331 | 0.0905 | −0.073 | 0.435 | −0.527 |
| GPC2 | 692352II [symbol] | −0.01 | 0.793 | −0.743 | 0.229 | 0.369 | −0.537 | 0.01 | −0.746 | −0.8665 | 0.985 | −0.085 | 0.984 | 0.1285 | 0.961 | −0.408 | 0.306 |
| GPR125 | 694724II [symbol] | −0.681 | −1.832 | −3.657 | −0.897 | −1.088 | −0.758 | 0.023 | −0.579 | −0.389 | −1.062 | −1.562 | −0.958 | −0.736 | −1.045 | −0.915 | −0.065 |
| GPR160 | 682327II [symbol] | 0.072 | 0.674 | 0.013 | −0.134 | 0.625 | −0.738 | −1.284 | −0.5005 | −0.2635 | 1.038 | 0.981 | −0.542 | 0.567 | −0.349 | −0.531 | 1.344 |
| GPR56 | 688865II [symbol] | −0.4095 | −0.5035 | 0.1555 | −0.5045 | −0.8545 | 0.0375 | 0.6995 | 0.2165 | −0.9795 | −0.1825 | 0.6255 | 0.3225 | −1.874 | −0.3485 | −0.2225 | 0.3475 |
| GPRC5C | 696709II [symbol] | −0.185 | −1.186 | 0.171 | −0.085 | −0.261 | −0.25 | −0.469 | −1.1765 | 0.543 | 1.571 | −0.917 | −1.255 | 0.235 | 0.539 | 0.539 | 0.018 |
| GPSM1 | 682326II [symbol] | 0.3987 | −0.0553 | −0.4903 | 0.3787 | −0.6883 | 0.1927 | 0.4277 | 0.8907 | −0.0428 | 0.5327 | 0.5257 | −0.1633 | −0.0443 | −0.5153 | 0.9147 | −0.3093 |
| GPSM2 | 690902II [symbol] | −0.943 | −0.969 | 0.227 | −0.005 | 1.542 | 0.932 | −1.306 | −0.296 | 0.352 | 0.182 | −0.467 | 1.614 | −0.2145 | −0.489 | 0.502 | 0.976 |
| GPT | 693608II [symbol] | −0.875 | −1.013 | −0.285 | −0.588 | 0.264 | −0.068 | 0.249 | −0.0315 | −0.1455 | 0.326 | 0.152 | 0.252 | 0.544 | 0.508 | −0.153 | −0.866 |
| GPT2 | 689304II [symbol] | −0.586 | −0.699 | −0.032 | 0.474 | −1.538 | −0.097 | 0.333 | −0.058 | −0.1925 | 0.169 | −1.05 | −0.941 | −1.458 | 1.156 | −1.456 | 0.039 |
| GPX7 | 694626II [symbol] | −0.104 | 0.041 | −1.039 | −0.187 | −0.818 | 0.207 | 0.106 | 0.2355 | −0.2935 | −0.73 | 0.713 | 0.501 | −0.065 | −0.034 | −0.167 | −0.238 |
| GRB14 | 691136II [symbol] | 3.215 | −0.1078 | −0.4608 | −1.39 | 0.7762 | 3.036 | 1.287 | −0.3648 | 2.536 | 4.131 | −0.4198 | −0.6808 | 0.0128 | −0.8318 | −0.3878 | −0.0128 |
| GRB7 | 691667II [symbol] | −0.304 | 0.181 | −0.228 | −0.376 | −0.29 | 2.53 | 0.193 | 2.368 | −0.0755 | 0.374 | −0.35 | 0.646 | 0.035 | −0.105 | 0.269 | 1.709 |
| GSC | 684798II [symbol] | 0.5113 | −0.3277 | 1.04 | 2.039 | 1.631 | −0.2397 | −0.6227 | 0.9548 | −0.0713 | 1.953 | 0.9113 | −0.4457 | −0.5827 | −0.1397 | 1.699 | −0.1007 |
| GSDML | 684490II [symbol] | 0.22 | −1.292 | 0.18 | −0.391 | −0.42 | −0.282 | 2.098 | −0.6895 | 2.018 | 0.847 | −0.388 | 0.488 | −1.9425 | −2.014 | 0.265 | 0.016 |
| GSK3B | 682653II [symbol] | −0.563 | 1.102 | −0.864 | −0.078 | 1.018 | −0.102 | 0.777 | 0 | 0.3205 | 0.618 | 1.056 | 1.09 | 2.017 | 1.203 | 0.224 | −0.284 |
| GSTA1 | 690111II [symbol] | 0.684 | −0.182 | 0.071 | −0.393 | 0.309 | 0.002 | 0.681 | −0.125 | −0.0585 | −0.234 | −0.354 | −0.057 | −0.409 | −0.124 | −0.175 | −0.086 |
| GSTK1 | 691113II [symbol] | −0.673 | −0.245 | 0.094 | −0.106 | 0.188 | 1.539 | −0.367 | 0.4655 | −0.05 | 0.496 | −1.378 | 0.385 | −0.3785 | −0.903 | −0.485 | −0.241 |
| GSTM3 | 686813II [symbol] | 2.189 | 2.646 | −0.445 | −1.643 | 1.021 | 2.856 | −1.926 | 0.006 | −0.1555 | 2.045 | 0.17 | −0.3 | 2.588 | −0.006 | 1.685 | −1.439 |
| GSTP1 | 690952II [symbol] | 0.204 | −0.303 | 1.78 | 0.438 | −0.456 | −0.546 | 1.124 | 0.485 | 0.459 | −0.348 | −0.434 | −1.455 | −0.903 | −0.992 | 0.76 | 1.139 |
| GSTT1 | 684076II [symbol] | −1.46 | −1.689 | −1.21 | −1.96 | −1.84 | 1.27 | −1.96 | −2.021 | −2.325 | −1.86 | −1.5 | −0.86 | −1.8855 | −1.39 | −2.212 | −0.94 |
| GSTT2 | 685053II [symbol] | 0.394 | 0.267 | −0.642 | 2.049 | 0 | 1.082 | −1.031 | 1.465 | −0.7965 | −0.613 | 0.162 | 0.269 | −0.006 | 1.196 | −0.0684 | 1.052 |
| GTF2F2 | 697621II [symbol] | −1.144 | −1.198 | −1.392 | 0.367 | −1.119 | −0.702 | 0.087 | −1.1935 | −0.822 | −0.486 | −0.038 | −0.963 | −0.8105 | 0.083 | −0.633 | −0.908 |
| GTF2I | 692303II [symbol] | 0.26 | 0.374 | −0.483 | 0.717 | 0.94 | −0.184 | −0.251 | −0.317 | 0.58 | 0.427 | 0.276 | 0.707 | 0.722 | 0.028 | 0.398 | −0.23 |
| GTF3A | 693886II [symbol] | −0.333 | −0.811 | 0.775 | −0.431 | 0.213 | 0.026 | 1.191 | −0.644 | −0.64 | 0.207 | 0.482 | 0.081 | −0.65 | −1.347 | −0.026 | 0.81 |
| GTPBP3 | 682310II [symbol] | −0.4755 | 0.0005 | 1.445 | −2.058 | −2.437 | −0.1345 | 0.1265 | 0.9495 | −1.456 | −1.758 | −0.8735 | −1.052 | −1.63 | 0.4095 | −0.7765 | 1.104 |
| GTPBP4 | 689649II [symbol] | −0.8405 | 0.0415 | 0.9405 | −0.1545 | −0.3895 | −0.0165 | 1.381 | −0.4155 | −1.471 | −0.0845 | 0.9365 | −0.1465 | −0.895 | −0.2725 | −0.2995 | 0.3515 |
| H1F0 | 692476II [symbol] | 0.1465 | 0.0525 | −0.2415 | 0.0005 | −0.0775 | −0.8455 | −0.0005 | −0.4405 | 0.071 | 0.1915 | −0.1965 | −0.1475 | −0.4155 | −0.2995 | −0.8295 | −0.0895 |
| H2AFV | 695317II [symbol] | −2.585 | −0.154 | 0.364 | −0.992 | −0.719 | −1.19 | −0.264 | −0.3195 | −1.1425 | −1.244 | −0.029 | −0.665 | −0.332 | −1.294 | −0.573 | −0.661 |
| H2AFY | 696406II [symbol] | −0.103 | 0.751 | 0.314 | 0.796 | 0.46 | 0.598 | 0.544 | 0.6815 | 0.0035 | 0.431 | 0.882 | 0.039 | 0.2915 | 0.339 | 0.329 | 0.695 |
| H2AFZ | 683032II [symbol] | −0.3055 | −0.0275 | 1.398 | 0.0035 | −0.3565 | −0.4685 | 0.4695 | −0.7455 | −1.496 | 0.6975 | 0.6985 | −0.3585 | −0.57 | −0.0645 | −0.6515 | 0.8365 |
| H2BFS | 685337II [symbol] | −1.384 | −0.5255 | 0.4525 | −0.8525 | −0.0625 | −0.3665 | 0.8125 | −0.6175 | 0.0105 | 0.7805 | 0.8815 | 0.7245 | 0.42 | 0.4025 | −0.1165 | −0.4655 |
| H3F3B | 690295II [symbol] | −0.584 | −0.152 | 0.086 | 0.078 | 0.004 | −0.741 | 0.737 | −0.325 | −0.7265 | 1.617 | 0.056 | 0.332 | −0.5085 | 0.073 | 0.244 | −0.168 |
| H41 | 682811II [symbol] | 0.1815 | −0.5035 | 1.357 | −0.5145 | −0.8105 | 0.0415 | 0.8045 | 0.3305 | −0.771 | −0.7865 | −0.0415 | −0.9025 | −1.048 | −0.8825 | 0.1315 | 0.7715 |
| HAGH | 696380II [symbol] | −0.739 | −0.057 | −0.252 | 0.864 | 0.652 | −0.717 | −0.091 | 0.2695 | 0.754 | 0.737 | −0.399 | −0.143 | −0.1275 | 0.92 | 0.488 | 0.257 |
| HBG1 | 688388II [symbol] | 0.226 | 0.341 | −0.369 | −0.212 | −0.14 | −0.076 | −0.466 | −0.134 | −0.431 | −0.097 | 0.467 | 0.098 | −0.2385 | −0.038 | −0.091 | 0.105 |
| HBLD1 | 697677II [symbol] | −0.771 | −0.528 | −0.514 | −0.251 | −0.214 | −0.05 | −1.063 | −0.991 | −0.857 | −0.411 | −0.591 | 0.43 | −0.202 | −0.588 | −0.037 | −0.366 |
| HCAP-G | 691867II [symbol] | −1.389 | −0.335 | 0.776 | −0.187 | −0.915 | 0.296 | 0.543 | 0.989 | 0.025 | −0.958 | 0.206 | −1.207 | −0.719 | −1.035 | −0.628 | −0.025 |
| HCP5 | 695382II [symbol] | 1.077 | 1.177 | 1.768 | 3.244 | 1.39 | 1.208 | 1.562 | 1.5825 | 1.14 | 2.509 | 2.105 | 0.963 | 0.6245 | 1.915 | 1.324 | 2.053 |
| HDAC2 | 697848II [symbol] | −1.423 | −0.939 | 0.007 | −0.956 | −1.234 | −0.34 | 1.177 | −0.9035 | −1.254 | −0.335 | −0.529 | −1.029 | −1.496 | −0.627 | −1.048 | −0.258 |
| HDC | 688061II [symbol] | 0.9595 | 1.546 | 0.6395 | 5.862 | −0.1125 | 0.2495 | −0.0505 | 0.199 | 2.602 | −0.0535 | 0.4255 | −0.1415 | −0.2605 | 5.683 | 0.2145 | 0.3745 |
| HEBP1 | 697035II [symbol] | 0.586 | 1.112 | −0.581 | 1.973 | 0.979 | 0.062 | −0.174 | −0.488 | 0.127 | 0.692 | 0.997 | 0.388 | 0.3475 | 0.641 | 0.161 | −0.122 |
| HIPK2 | 691802II [symbol] | 0.1025 | 0.1115 | −0.3095 | 0.1155 | 0.0025 | 0.0705 | −0.0025 | 0.4565 | 1.08 | −0.0645 | −0.3115 | −0.3165 | −0.158 | −0.1375 | −0.4955 | −0.2425 |
| HIPK2 | 694742II [symbol] | −0.136 | 1.835 | 0.1 | 2.139 | 1.676 | 1.645 | 0.808 | 2.1685 | 3.195 | 0.511 | 0.803 | 2.068 | 1.2145 | 1.696 | 1.307 | 0.574 |
| HIS1 | 694010II [symbol] | −0.539 | 0.54 | 1.027 | 0.46 | 1.357 | −0.168 | −0.366 | −0.1065 | 0.6005 | 1.565 | 0.049 | 0.889 | −0.103 | 0.729 | 0.469 | 0.87 |
| HIST1H2A | 684611II [symbol] | −2.959 | 0.8663 | −0.5437 | 0.1193 | 0.4083 | −0.4557 | 0.6543 | −0.2727 | −0.0073 | 0.3283 | −0.2787 | 0.3763 | 0.2043 | 0.0073 | 0.2423 | −1.269 |
| HIST1H2B | 697885II [symbol] | 0.0408 | 3.604 | −3.185 | 3.19 | 4.385 | −2.618 | −2.273 | −3.393 | 2.701 | 2.937 | 1.041 | 2.828 | 3.185 | 2.859 | 1.467 | −0.408 |
| HIST1H2B | 681566II [symbol] | −1.745 | −0.21 | 0.094 | −0.583 | 0.044 | −0.121 | 0.931 | −0.9565 | −0.426 | 0.954 | 0.629 | 0.36 | 0.046 | 0.489 | 0.418 | −0.6 |
| HIST1H2B | 695646II [symbol] | −1.895 | −0.159 | 0.31 | −0.499 | 0.215 | −0.233 | 0.741 | −0.8165 | −0.1895 | 0.81 | 0.432 | 0.099 | 0.7435 | 0.397 | 0.292 | 0.836 |
| HIST1H2B | 683833II [symbol] | −2.167 | 0.0145 | 0.2255 | −0.8535 | 0.0225 | −0.3185 | 0.7095 | −0.8215 | −0.4075 | 0.7725 | 0.4706 | 0.2045 | 0.7025 | 0.2065 | 0.0955 | −0.8305 |
| HIST1H3C | 693912II [symbol] | −0.441 | −0.303 | −0.317 | −0.631 | −0.775 | −0.871 | 0.237 | −0.6355 | −1.2085 | 1.113 | −0.235 | 0.088 | −0.5655 | −0.342 | −0.158 | −0.423 |
| HM13 | 695097II [symbol] | −0.884 | 0.043 | 0.834 | 0.494 | −0.016 | 0.646 | 0.132 | 0.3625 | −0.6925 | −0.406 | −0.07 | −0.504 | −0.5645 | 0.193 | −0.316 | −0.063 |
| HMGB3 | 690848II [symbol] | 0.5 | 1.455 | 0.666 | −0.258 | −0.296 | 0.789 | 1.733 | −1.651 | 0.294 | 3.69 | 0.387 | 2.063 | 2.172 | 0.25 | 0.922 | 2.339 |
| HN1 | 689578II [symbol] | −1.78 | −0.482 | 1.981 | −0.173 | −0.293 | −0.646 | 1.523 | 0.757 | −0.2695 | 0.087 | 0.135 | −0.404 | −0.5715 | −0.375 | −0.685 | −1.037 |
| HNRPA3P | 687868II [symbol] | −0.7775 | −0.1455 | 0.8365 | 0.1205 | 0.3795 | 0.3555 | 0.7395 | 0.0015 | −0.103 | −0.0045 | −0.1445 | 0.4725 | −0.48 | −0.0015 | 0.2705 | 0.4775 |
| HNRPDL | 689713II [symbol] | −0.2693 | −0.0473 | −0.9523 | −0.0933 | 0.2997 | −0.0183 | −0.2053 | 0.0183 | −0.7298 | 0.5737 | 0.2197 | −0.0743 | −0.4778 | 0.2407 | −0.0633 | 0.3787 |
| HOXA5 | 682213II [symbol] | 0.755 | 0.019 | −0.53 | 0.352 | −0.074 | 0 | 0.051 | 1.254 | −0.1125 | −0.386 | −0.476 | −0.158 | 0.394 | 0.447 | 0.093 | 0.111 |
| HOXA7 | 693224II [symbol] | 1.233 | 0.384 | −1.481 | 0.408 | 0.02 | 0.371 | −0.833 | 2.811 | 0.5405 | 0.124 | −0.179 | −0.108 | −0.0405 | 0.26 | 0.899 | 0.497 |
| HOXB2 | 695779II [symbol] | 0.706 | 0.757 | −1.138 | 0.816 | 1.083 | 0.68 | −1.168 | 1.3575 | 0.3515 | 1.611 | 1.48 | 2.69 | 1.364 | 2.145 | 0.91 | 2.507 |
| HOXB6 | 688928II [symbol] | −0.719 | −1.966 | −0.754 | −1.461 | −1.77 | −1.309 | −0.514 | −0.136 | −1.826 | −1.632 | −0.884 | −1.221 | −1.267 | −0.382 | −0.683 | −0.409 |
| HOXB7 | 687026II [symbol] | −1.68 | −0.86 | 1.146 | −0.016 | −0.171 | 0.286 | 0.202 | 1.275 | 0.499 | −1.402 | −0.316 | 1.589 | 0.5925 | 0.357 | 0.875 | 1.202 |
| HOXC10 | 688125II [symbol] | −1.285 | 1.285 | 0.4495 | 0.3225 | 0.7515 | 2.216 | 0.8195 | 1.948 | −0.488 | −1.581 | 1.529 | 0.8785 | −1.816 | −1.814 | 1.541 | 0.5825 |
| HOXC13 | 688022II [symbol] | −0.6865 | 0.1705 | −0.1295 | 0.3795 | 0.0885 | 0.3405 | 0.1315 | 0.1445 | −0.1425 | 0.1185 | −0.1455 | −0.1295 | −0.6255 | 0.2955 | 0.8535 | −0.1145 |
| HOXC9 | 686974II [symbol] | −0.7 | −0.846 | 0.179 | −0.766 | −1.081 | 0.733 | 0.145 | 0.613 | −0.2605 | −0.64 | −0.256 | −0.732 | −0.5035 | −1.048 | −0.173 | −0.763 |
| HOXD3 | 688881II [symbol] | −1.49 | −1.339 | −0.3795 | −0.1945 | 0.3585 | 0.0115 | 0.2605 | −0.1555 | −0.117 | 0.3595 | 0.2465 | 0.7245 | 0.67 | 0.5535 | 0.1065 | −0.5945 |
| HPCL2 | 693166II [symbol] | −0.96 | −0.267 | −0.844 | −0.712 | −0.307 | −1.093 | −1.434 | −0.795 | −0.5265 | −0.347 | −1.056 | −0.234 | −0.2535 | −0.295 | −1.038 | −0.898 |
| HPS3 | 684956II [symbol] | −0.243 | −0.034 | −0.489 | 0.187 | 0.25 | −0.293 | −0.003 | −0.675 | −0.295 | 0.727 | 0.707 | 0.737 | 0.5285 | 0.047 | −0.416 | 0.337 |
| HRASLS | 689305II [symbol] | −1.17 | −0.182 | 2.368 | 2.154 | 0.774 | −0.146 | 1.603 | 1.194 | 0.3155 | 0.093 | 0 | −1.167 | −1.23 | 0.113 | −0.078 | −1.059 |
| HRB | 692071II [symbol] | −0.5825 | −0.0295 | 0.3825 | −0.6225 | −0.5035 | −0.1895 | 0.3425 | −0.1455 | −0.7005 | 0.5565 | 0.6195 | 0.2305 | −0.489 | −0.2045 | 0.0295 | 0.7925 |
| HRSP12 | 681060II [symbol] | −1.175 | −0.391 | −0.226 | −0.799 | −0.638 | 0.539 | 0.029 | −0.003 | −0.901 | 0.014 | −0.499 | 0.378 | −0.1405 | −0.248 | −1.155 | 0.257 |
| HSD17B2 | 692999II [symbol] | −0.782 | −0.999 | 0.594 | −0.953 | −1.517 | 1.824 | 0.515 | −0.794 | −1.0145 | −1.521 | −0.997 | −0.746 | −1.415 | −2.314 | −1.229 | −0.496 |
| HSF1 | 694939II [symbol] | 0.412 | 1.115 | −1.762 | −1.114 | −0.993 | 0.001 | −0.095 | 0.517 | 0.8035 | 0.026 | 0.888 | 0.427 | 1.119 | 1.144 | 1.929 | 0.524 |
| HSPA14 | 694810II [symbol] | −0.21 | −0.138 | −0.347 | 0.01 | 0.364 | −0.38 | −0.52 | −0.1045 | 0.7 | 0.353 | 0.077 | 0.378 | 0.2855 | −0.157 | −0.132 | 0.177 |
| HSPA2 | 695880II [symbol] | 0.526 | 1.859 | 0.85 | 1.908 | 1.614 | 1.012 | 0.139 | 0.176 | 2.052 | 2.906 | 1.565 | 0.837 | 1.306 | 1.378 | 2.046 | 0.577 |
| HSPA5BP | 693776II [symbol] | 0.465 | −0.613 | 0.168 | 1.938 | −0.57 | −0.144 | 1.093 | 0.0865 | 0.6215 | 0.087 | −0.828 | −0.762 | −0.403 | −0.136 | 0.895 | −0.045 |
| HSPC016 | 688206II [symbol] | −0.8075 | 0.0545 | −0.1305 | 0.1775 | 0.1655 | −0.0025 | −0.0565 | −0.7745 | 0.592 | 0.6585 | 0.0285 | 0.1685 | 0.1535 | 0.2505 | 0.1215 | 0.1585 |
| HSPC138 | 696635II [symbol] | −1.266 | −1.31 | −0.883 | −0.652 | −0.606 | −0.907 | −1.28 | −1.3565 | −2.0075 | −0.568 | −0.416 | −0.631 | −1.04 | −0.39 | −0.834 | −0.938 |
| HSPC148 | 687877II [symbol] | −0.981 | −0.539 | −0.306 | 0.21 | 0.106 | 0.399 | 0.598 | 0.001 | 0.0445 | −0.534 | −0.053 | −0.054 | −0.161 | 0.007 | −0.355 | 0.09 |
| HSFC150 | 692010II [symbol] | −1.315 | −0.2065 | 1.367 | −1.048 | −0.5775 | 0.0635 | 0.6735 | 0.117 | −0.359 | −0.0635 | 0.6385 | 0.1835 | 0.1465 | −0.9305 | −0.4355 | 0.5315 |
| HSPC183 | 691987II [symbol] | −1.441 | −0.608 | −0.348 | −1.095 | −0.819 | 0.415 | 0.239 | −0.014 | −0.095 | −0.961 | −0.161 | 0.432 | −0.226 | −1.064 | −0.819 | 0.348 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| HSPC268 | 693184II [symbol] | −1.161 | −0.419 | 0.195 | −0.325 | 0.043 | −0.396 | −0.126 | −0.086 | 0.4685 | 0.208 | −0.716 | −0.362 | 0.2405 | 0.859 | −0.368 | −0.544 |
| HSU7927 [symbol] | 693843II [symbol] | 0.382 | 0.184 | −0.054 | −0.021 | 0.095 | 0.118 | 0.157 | −0.0635 | 0.6215 | −0.463 | −0.341 | 0.222 | 0.2015 | −0.335 | −0.086 | −0.27 |
| HTATIP2 | 693716II [symbol] | 0.082 | −0.162 | −1.127 | 0.03 | −0.869 | −0.051 | 0.609 | −0.2305 | −0.6755 | −0.429 | 0.558 | 0.069 | −0.54 | 0.025 | 0.082 | 0.299 |
| ICMT | 681046II [symbol] | −1.316 | 0.1965 | 0.7665 | −0.6665 | −0.3965 | −0.4225 | 0.3845 | 0.783 | 0.4165 | −0.1065 | −0.0125 | −0.1555 | −0.726 | 0.0515 | −0.2185 | 0.0515 |
| ICT1 | 683297II [symbol] | −1.279 | −0.3945 | 0.2175 | 0.0205 | 0.0935 | −0.6575 | 0.4645 | −0.477 | 0.7915 | 0.8285 | −0.5885 | 0.2385 | 0.1205 | 0.0025 | −0.2215 | −0.3885 |
| ID4 | 685578II [symbol] | −0.0015 | 0.2155 | 0.0485 | 0.6365 | 0.3025 | 0.3545 | −0.3545 | 0.0765 | 0.1435 | −0.2675 | −0.3645 | −0.5315 | 0.211 | −0.0325 | −0.3845 | −0.4605 |
| IDH2 | 681706II [symbol] | −0.822 | 0.791 | 1.097 | −0.07 | −0.637 | 2.602 | 0.833 | 1.588 | −0.6705 | −0.666 | 0.542 | −0.107 | 1.04 | −1.318 | −0.745 | 1.98 |
| ID5 | 696420II [symbol] | 0.051 | −0.715 | −1.01 | −0.245 | −0.559 | −1.16 | 2.938 | −0.8145 | −0.314 | −0.166 | −0.378 | −0.433 | −0.72 | −0.699 | −0.523 | 0.922 |
| IFI30 | 695270II [symbol] | 0.383 | 1.353 | 2.757 | 1.717 | 1.099 | 1.688 | 1.587 | 2.496 | 1.5135 | 0.722 | 1.82 | 0.638 | 2.072 | 1.334 | 0.915 | 1.929 |
| IFI44 | 681212II [symbol] | 0.077 | 0.126 | −0.368 | 0.086 | 0.266 | −1.076 | −0.491 | −0.341 | 0.127 | 0.629 | −0.482 | 0.405 | −0.132 | −0.328 | −0.276 | 0.053 |
| IFIT5 | 684503II [symbol] | −0.5042 | −0.2722 | 0.8778 | −0.7092 | −0.1902 | 0.3318 | 0.4968 | 0.0163 | −0.7247 | −0.0163 | −0.1402 | 0.6268 | −0.2852 | −0.2872 | −0.4832 | 0.5918 |
| IFITM1 | 690666II [symbol] | −0.288 | −0.177 | 0.454 | 0.179 | −1.01 | 0.048 | 0.834 | 0.9875 | −0.5805 | 0.276 | 1.145 | −0.64 | −1.493 | 0.572 | −0.026 | 0.459 |
| IGBP1 | 697847II [symbol] | −0.174 | −0.169 | −0.594 | 0.469 | 0.358 | −0.391 | −0.809 | −0.3875 | 0.3905 | 0.04 | −0.504 | 0.571 | 0.654 | 0.246 | −0.018 | −0.021 |
| IGFALS | 692644II [symbol] | 0.615 | 0.515 | −0.339 | 2.748 | 2.083 | −0.08 | 0.361 | 0.1805 | 3.505 | 0.457 | 1.235 | 1.653 | 1.5395 | 2.696 | 1.444 | −0.209 |
| IGFBP2 | 689980II [symbol] | 0.0505 | 0.8165 | −1.757 | −1.187 | 0.8785 | 1.385 | −0.3715 | −1.094 | 0.657 | 0.5005 | −1.314 | −1.225 | 0.9455 | −0.9015 | −0.3365 | −0.1095 |
| IGHG1 | 682030II [symbol] | 0.408 | 0.082 | 0.021 | 0.539 | 1.052 | 0.856 | 0.601 | 0.977 | 0.001 | 2.174 | 0.609 | −0.103 | −0.3685 | −0.599 | −0.445 | −0.293 |
| IGHG1 | 696457II [symbol] | 0.24 | 1.028 | 0.577 | 1.93 | 1.914 | 0.839 | 0.684 | 0.7135 | 0.416 | 1.973 | 0.851 | 0.334 | 0.109 | 0.214 | 0.279 | 0.675 |
| IGJ | 687674II [symbol] | 0.9615 | −1.09 | −1.135 | 0.7325 | 0.6775 | −0.2155 | 1.104 | −0.6835 | 1.565 | 2.208 | −0.5615 | 0.5415 | −1.604 | −2.104 | 0.7665 | 1 |
| IGKC | 690921II [symbol] | −1.119 | −0.61 | −0.896 | 0.73 | 0.716 | −0.697 | −0.729 | 1.359 | −0.154 | 1.768 | 0.184 | 0.154 | −0.9235 | −0.744 | −0.688 | −0.2 |
| IGKC | 691031II [symbol] | −0.345 | −0.16 | −0.308 | 0.503 | 1.057 | −0.049 | 0.049 | 0.67 | 0.0935 | 0.828 | 0.615 | −0.23 | −0.339 | −0.403 | −0.1 | −0.191 |
| IGLC2 | 682716II [symbol] | −0.1005 | −0.1415 | −0.3405 | 0.1985 | 0.1425 | 0.3945 | −0.1175 | 1.017 | 0.917 | 0.1605 | −0.0345 | 0.0655 | −0.0485 | −0.3035 | −0.1455 | −0.0065 |
| IGLL1 | 691836II [symbol] | 1.466 | −0.464 | −0.877 | 0.862 | 0.635 | 0.66 | 0.167 | 0.746 | −0.7375 | 1.1 | −0.118 | −0.657 | −1.407 | −1.028 | 0.267 | −0.488 |
| IL13RA1 | 697838II [symbol] | −0.355 | 0.014 | 0.548 | −0.267 | −0.147 | 0.287 | 0.641 | −0.0655 | −0.0595 | 0.207 | 0.446 | 0.671 | 0.0495 | 0.602 | 0.48 | 0.774 |
| IL17B | 685612II [symbol] | 0.2985 | 0.4465 | −0.1965 | 0.0685 | −0.3275 | 0.1645 | −0.0995 | −0.2575 | 0.1845 | 1.656 | 0.6065 | −0.0855 | −0.353 | 0.0515 | 0.3965 | 0.6345 |
| IL23A | 690046II [symbol] | 0.304 | −0.417 | 0.953 | 0.123 | −0.291 | 0.045 | 0.733 | −0.045 | 0.3115 | −0.156 | −0.454 | −0.529 | −0.124 | −0.244 | −0.208 | −0.334 |
| IL6ST | 690686II [symbol] | −0.0715 | 0.6885 | −0.8905 | 1.765 | 2.795 | −0.7135 | −0.4255 | −1.359 | 0.9375 | 1.621 | 1.228 | 2.031 | 1.163 | 0.7245 | 1.175 | 0.9195 |
| ILF2 | 681204II [symbol] | 0.318 | 1.423 | 0.228 | 0.482 | 2.526 | −0.186 | 0.682 | 0.492 | −0.0025 | 3.42 | 0.999 | 0.315 | 0.35 | 0.477 | 0.601 | 0.544 |
| IMPA2 | 690416II [symbol] | 0.671 | −0.459 | 1.891 | 0.244 | −0.876 | 0.339 | 0.726 | 0.504 | −0.579 | −1.12 | −1.11 | 0.892 | −0.1205 | −0.126 | −0.026 | 0.026 |
| ING1L | 681976II [symbol] | −1.137 | 0.1775 | 0.1205 | −0.1895 | 0.0645 | −0.2645 | 0.0845 | −0.3655 | −0.332 | 0.9895 | −0.4155 | 0.6005 | 0.063 | −0.4155 | 0.2505 | 0.0005 |
| INHBB | 695995II [symbol] | 2.147 | 2.512 | 1.919 | 2.795 | 2.045 | 3.734 | 2.164 | 3.3615 | 1.5385 | 1.273 | 1.294 | 3.434 | 0.696 | 1.789 | 2.748 | 3.616 |
| INPP4B | 685012II [symbol] | −0.097 | 1.952 | −1.538 | −0.32 | 1.688 | −0.335 | −1.24 | −1.332 | −0.394 | 1.414 | 0.878 | 2.168 | 0.7665 | −0.798 | 0.712 | 0.763 |
| INSM1 | 693435II [symbol] | 1.848 | 2.529 | −0.19 | −0.466 | 2.355 | −0.042 | −0.261 | −0.072 | −0.178 | 0.881 | −0.176 | −0.338 | 1.5595 | 5.923 | 1.577 | 0.344 |
| IQCA | 692963II [symbol] | 1.075 | 3.039 | −0.107 | 0.439 | 3.225 | 1.699 | 2.19 | 1.911 | 1.1465 | 2.193 | 0.974 | 1.848 | 0.1905 | 1.016 | 2.643 | 1.774 |
| IQCE | 687430II [symbol] | −0.3265 | −0.2725 | −0.1865 | −0.0565 | 0.6285 | 0.1335 | −0.4985 | 0.764 | 0.033 | −0.2775 | 0.5555 | 0.7505 | 0.158 | −0.0605 | −0.0645 | 0.8425 |
| IQGAP2 | 697172II [symbol] | −0.1915 | 0.0095 | 0.2355 | −0.1225 | −0.1005 | 0.2455 | 0.2845 | −0.1455 | −0.3775 | 0.2135 | 0.8075 | 0.2015 | −0.7015 | −0.3505 | 0.0105 | 0.4795 |
| IRF4 | 690581II [symbol] | 0.9585 | −0.5535 | 0.5825 | 0.5355 | 0.5855 | 0.8515 | −0.1525 | 0.1545 | 0.084 | 0.1405 | −0.4285 | −0.6695 | −0.846 | −0.2785 | 0.1096 | 0.6645 |
| IRTA2 | 686673II [symbol] | 0.925 | −0.354 | −0.87 | 0.446 | 1.138 | 1.025 | 0.166 | 0.4285 | −0.4935 | 0.384 | −0.093 | −0.756 | −0.7215 | −0.721 | −0.244 | 0.551 |
| IRX4 | 682616II [symbol] | 1.145 | 0.06 | −1.45 | 0.076 | −0.069 | −1.748 | −1.997 | −1.122 | −0.2305 | 0.258 | −0.06 | 3.15 | −1.215 | −2.277 | 0.367 | 0.317 |
| ISG20 | 689588II [symbol] | 0.7225 | 0.3845 | −0.7445 | −0.1485 | 0.1465 | −0.1665 | −0.2505 | 0.145 | −0.6595 | 0.5205 | 0.1745 | 0.6905 | 0.118 | 0.0285 | −0.0005 | 0.2875 |
| ISGF3G | 684521II [symbol] | 0.319 | 0.697 | 2.39 | 0.514 | 1.163 | 0.213 | 3.567 | 1.516 | 0.342 | 0.467 | 0.496 | 0.919 | 0.2255 | 0.569 | 0.592 | 0.939 |
| ITGB4 | 692936II [symbol] | 0.633 | 1.216 | −0.099 | 4.823 | 0.193 | 0.059 | 0.294 | 0.4685 | 1.232 | 4.219 | 3.56 | 0.441 | 0.2075 | 1.631 | 0.656 | 1.717 |
| ITPR1 | 690279II [symbol] | 1.822 | 1.036 | −0.7155 | 1.58 | 1.557 | −0.4835 | −1.252 | −1.686 | −1.016 | 0.9045 | −0.4905 | 0.9435 | 0.0515 | 1.264 | −0.4445 | 0.2535 |
| JARID1A | 694293II [symbol] | 0.553 | 0.413 | 0.315 | 0.85 | 0.815 | 0.841 | 0.789 | 0.7715 | −0.001 | 0.609 | 0.227 | 0.183 | 0.5635 | 0.289 | 0.971 | 0.25 |
| KARCA1 | 692006II [symbol] | −0.929 | −0.128 | −1.706 | −0.65 | 1.723 | −0.627 | −1.221 | −1.345 | 0.7255 | −0.119 | −0.056 | 2.049 | 1.188 | 0.177 | 0.063 | −0.106 |
| KATNA1 | 692605II [symbol] | −0.723 | −0.109 | 0.572 | −0.331 | −0.077 | −0.168 | −0.495 | 0.179 | −0.492 | −0.547 | −0.548 | −0.595 | −0.6235 | −0.978 | −0.291 | −0.15 |
| KCNC2 | 686570II [symbol] | −0.4325 | −0.2095 | −0.0045 | −0.0885 | 0.0405 | −0.0145 | −0.1115 | −0.1135 | 0.176 | 0.7085 | −0.2225 | 0.0045 | −0.236 | 0.4835 | 0.0165 | −0.1525 |
| KCND2 | 683134II [symbol] | −0.935 | −0.305 | 0.234 | 3.532 | 0.254 | −0.241 | 0.04 | 0.3265 | 0.07 | −0.169 | 1.023 | 0 | −0.485 | 2.908 | 0.706 | 0.217 |
| KCNK4 | 691761II [symbol] | −0.219 | −0.043 | −0.321 | 0.29 | 0.03 | 0.164 | −0.015 | 0.3865 | 0.9375 | 0.13 | −0.308 | −0.317 | −0.2315 | 0.0365 | −0.042 | −0.443 |
| KCNN4 | 692275II [symbol] | −0.3045 | −0.2495 | 0.3755 | −0.0975 | 0.1375 | 0.2555 | −0.2625 | 0.385 | 0.299 | −0.6045 | 0.0075 | −0.2075 | 0.0515 | 0.9145 | −0.3345 | 0.1495 |
| KCNS3 | 681093II [symbol] | −1.14 | −0.6875 | 0.2625 | −0.7165 | 0.2015 | 1.241 | 0.7615 | 0.13 | −0.7325 | −0.6065 | −0.8995 | 0.8645 | 0.0395 | 2.71 | −1.174 | 0.1115 |
| KCTD15 | 689199II [symbol] | −1.154 | 0.8345 | 0.0825 | −1.091 | −0.0945 | 0.1585 | −0.9515 | 0.654 | 0.5915 | −0.1615 | −1.096 | 1.119 | 0.627 | 0.4715 | 0.0095 | −0.4985 |
| KCTD3 | 686312II [symbol] | 0.316 | 0.034 | −0.725 | 0.083 | 1.443 | −0.023 | −0.626 | −0.545 | −0.587 | 0.362 | 0.058 | 1.647 | 0.9325 | −0.53 | 0.604 | −0.038 |
| KDELR2 | 697248II [symbol] | −2.168 | −0.188 | 0.623 | −1.287 | −1.835 | −0.311 | −0.142 | −0.0455 | −1.391 | −0.139 | 0.841 | 0.16 | −0.2145 | −1.079 | −0.188 | −0.612 |
| KIAA0020 | 696703II [symbol] | −1.024 | −0.996 | −0.915 | −1.1492 | −1.391 | −0.675 | −0.605 | −0.747 | −1.194 | −1.288 | −0.669 | −0.828 | −1.0375 | −0.974 | −1.288 | −0.786 |
| KIAA0101 | 691153II [symbol] | −2.049 | 0.2895 | 1.542 | −0.4445 | −0.0795 | −0.3635 | 0.3325 | −0.5245 | −0.8845 | −1.178 | 0.7285 | −0.3005 | 0.1165 | −0.8245 | −0.7485 | 0.6585 |
| KIAA0153 | 694669II [symbol] | −0.34 | 0.326 | 1.108 | 0.07 | 0.463 | 0.542 | 0.308 | 0.467 | 0.244 | −0.948 | 0.077 | 0.118 | 1.161 | 0.203 | 0.149 | −0.006 |
| KIAA0182 | 691550II [symbol] | −1.481 | −0.1833 | 0.4457 | 0.6397 | 0.2367 | 0.3287 | −0.7653 | −0.2813 | −0.0418 | 1.202 | 0.3177 | 0.0518 | −1.229 | 0.0418 | 0.6157 | 0.2637 |
| KIAA0196 | 685973II [symbol] | −0.2845 | 0.5405 | −0.1625 | −0.6645 | −0.2355 | −0.3695 | −0.5605 | −0.3915 | −0.711 | 0.2185 | 0.1415 | 0.5435 | 0.0145 | −0.1625 | −0.4275 | 1.007 |
| KIAA0241 | 692587II [symbol] | −1.356 | −1.126 | 0.216 | −0.977 | −1.107 | −0.844 | 0.055 | 0.2045 | −0.3335 | −0.66 | 0.145 | −0.584 | −0.474 | −1.412 | −0.508 | −0.414 |
| KIAA0251 | 682227II [symbol] | −0.9535 | 0.4905 | 0.2465 | −0.6415 | 0.2045 | 0.0365 | −0.1795 | −0.043 | −0.694 | −0.1665 | −0.2165 | 0.6515 | 0.0735 | −0.5595 | 0.3625 | 1.036 |
| KIAA0274 | 683166II [symbol] | 0.1255 | 0.2905 | 0.7055 | −0.1195 | −0.1105 | −0.7125 | −0.1495 | −0.9155 | −0.732 | −0.0455 | 0.3415 | 0.2515 | −0.628 | 0.4845 | −0.3705 | 0.0235 |
| KIAA0515 | 688133II [symbol] | 0.18 | 0.642 | 0.65 | −0.071 | 0.254 | −0.001 | −0.228 | 0.606 | −0.4565 | 0.075 | 0.133 | −0.303 | −0.313 | −0.126 | 0.064 | 0.142 |
| KIAA0582 | 697645II [symbol] | 1.047 | 1.236 | −0.667 | 0.406 | 1.222 | 0.788 | 0.627 | 1.6665 | 1.8815 | 0.884 | 0.592 | 1.165 | 1.0665 | 1.064 | 0.9 | 0.869 |
| KIAA0650 | 684609II [symbol] | −0.0595 | −0.5925 | 0.6025 | −0.0015 | 0.0015 | 0.2105 | 0.7385 | −0.523 | −0.5965 | 0.5675 | 0.0355 | 0.0535 | −0.342 | −0.2795 | −0.4245 | 0.3645 |
| KIAA0703 | 684139II [symbol] | −0.8435 | 0.1905 | 0.2995 | 1.175 | −0.2415 | 1.251 | 2.135 | 0.5875 | −0.232 | 1.21 | −0.9285 | −1.774 | −0.0835 | −0.3255 | −0.2915 | 1.072 |
| KIAA0830 | 692561II [symbol] | 1.026 | 0.052 | 0.293 | 1.749 | 1.95 | 1.708 | 1.158 | 1.0585 | 1.4795 | 1.01 | 0.398 | 1.499 | 0.208 | 2.018 | 0.835 | 1.045 |
| KIAA0980 | 686300II [symbol] | −0.124 | 0.75 | −0.614 | 0.398 | 0.383 | 1.184 | 1.034 | 1.073 | 0.501 | −0.123 | −1.294 | 0.042 | −0.313 | −0.851 | 0.159 | −0.261 |
| KIAA1324 | 690969II [symbol] | −0.4178 | 0.7782 | 0.2332 | −0.3818 | 0.7792 | 0.6022 | −0.6878 | −0.7923 | −0.0243 | 0.5782 | 0.0243 | 0.4472 | 0.2822 | 1.527 | 0.0993 | −0.4308 |
| KIAA1363 | 696389II [symbol] | −0.39 | −0.24 | 0.576 | −0.397 | 0.202 | −0.97 | −0.645 | 0.2415 | −0.309 | 0.037 | 0.145 | −0.562 | 0.543 | −0.135 | −0.535 | 0.456 |
| KIAA1683 | 684555II [symbol] | 0.6475 | 0.5255 | 1.235 | 0.0145 | −0.1375 | 0.6595 | 0.7675 | 0.362 | 0.4405 | 1.717 | 0.4415 | 1.692 | −0.151 | −0.2215 | 0.8625 | −0.0735 |
| KIF13B | 689768II [symbol] | −0.131 | −0.089 | −1.531 | 0.149 | 1.88 | −0.423 | −1.264 | −1.097 | 1.732 | 0.913 | 0.32 | 0.906 | 1.307 | 0.962 | −0.298 | −0.43 |
| KIF24 | 692033II [symbol] | 0.021 | −0.025 | 0.138 | −0.497 | 0.776 | 0.489 | 0.047 | −0.076 | 0.1475 | −0.236 | −0.287 | −0.283 | −0.071 | −0.575 | 0.205 | −0.432 |
| KIN | 687329II [symbol] | −0.434 | −0.303 | 0.389 | −0.589 | 0.136 | 0.227 | 1.257 | −0.0445 | −0.4445 | 0.01 | 0.767 | 0.213 | −0.39 | 0.056 | −0.164 | 0.249 |
| KIRREL3 | 693623II [symbol] | −0.054 | −0.311 | 0.055 | 0.193 | −0.073 | 1.963 | 0.541 | 0.354 | 0.8065 | 0.037 | 0.141 | −0.391 | 0.212 | −0.014 | −0.337 | −0.043 |
| KIT | 692519II [symbol] | 3.103 | 0.642 | −0.176 | 0.316 | −0.885 | 1.751 | 1.63 | −0.172 | 0.1205 | −0.508 | 0.656 | 0.284 | −0.018 | −0.467 | 0.75 | 1.73 |
| KLK5 | 693542II [symbol] | 0.86 | 0.893 | 0.746 | 0.589 | 0.091 | 1.126 | 4.053 | 1.1005 | 1.1535 | 1.625 | 1.465 | 0.65 | 0.409 | 0.12 | 0.745 | 1.848 |
| KLK6 | 684796II [symbol] | 0.9013 | −0.0118 | 0.9243 | −0.6537 | −1.431 | −1.086 | 4.963 | −0.9952 | 0.0118 | 0.7233 | 0.9123 | −0.7627 | −0.6937 | −1.117 | 0.4533 | 0.9943 |
| KLK7 | 695787II [symbol] | 0.381 | 0.729 | 0.881 | 0.276 | −0.402 | −0.335 | 4.147 | −0.0875 | 0.221 | 1.52 | 1.42 | 0.333 | −0.3595 | −0.123 | 0.493 | 1.861 |
| KLK8 | 688892II [symbol] | 0.7015 | 0.2295 | 0.8635 | 0.9265 | −0.4345 | 0.0195 | 3.697 | −0.23 | 0.4285 | 0.0525 | −0.2885 | −0.3965 | −0.176 | −0.2895 | 0.4435 | 1.127 |
| KNTC2 | 694720II [symbol] | −3.755 | −2.206 | −0.659 | −2.157 | −2.285 | −1.867 | −1.029 | −1.137 | −2.48 | −2.977 | −2.164 | −2.853 | −2.3395 | −2.745 | −2.464 | −1.519 |
| KPNA2 | 689153II [symbol] | −0.7125 | −0.7765 | 0.5645 | −0.0685 | −0.4175 | 0.6195 | 1.571 | 1.817 | −0.6155 | 0.9155 | −0.1415 | −0.4625 | −0.662 | −0.4085 | −0.2995 | 0.9025 |
| KREMEN [symbol] | 687365II [symbol] | 0 | −1.427 | −0.364 | −0.681 | −0.361 | 0.062 | −0.504 | 0.403 | −0.2125 | −0.929 | −0.428 | 0.707 | 0.026 | −1.449 | 0.355 | 0.864 |
| KRT10 | 694359II [symbol] | −1.197 | −1.32 | −1.437 | −1.324 | −0.835 | −0.182 | −1.228 | −1.588 | −1.0915 | −0.647 | −1.008 | −0.347 | −0.2025 | −1.203 | −0.597 | −1.074 |
| KRT13 | 696433II [symbol] | 2.123 | 1.716 | 1.809 | 1.221 | 3.075 | 0.203 | 1.911 | −0.119 | 0.531 | 0.335 | −0.184 | 0.867 | −0.3845 | −0.683 | 0.742 | 1.562 |
| KRT15 | 694764II [symbol] | −0.583 | −0.115 | −0.143 | −0.126 | −0.061 | 0.018 | 0.937 | −0.0395 | −0.018 | 0.988 | −0.021 | 0.147 | 0.489 | 0.17 | 0.181 | −0.504 |
| KRT17 | 690706II [symbol] | 1.604 | 0.383 | 1.188 | 0.041 | −1.028 | −0.538 | 1.218 | −0.519 | −1.304 | 0.62 | 0.087 | −0.81 | −1.667 | −1.359 | 1.049 | 0.464 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| KRT18 | 689283II [symbol] | −1.697 | 0.661 | −0.218 | −0.441 | 0.171 | −0.105 | −0.65 | 0.268 | 0.061 | −0.244 | 0.919 | −0.227 | −0.278 | 0.132 | 0.444 | 0.491 |
| KRT23 | 689793II [symbol] | −0.353 | 1.7 | −1.587 | −2.298 | 2.736 | 1.998 | 2.755 | 0.204 | −0.7495 | −2.769 | −3.09 | 2.078 | −2.111 | −0.992 | −1.109 | 0.267 |
| KRT6B | 696954II [symbol] | 1.542 | 2.236 | 3.666 | 2.236 | 0.616 | −0.468 | 5.529 | 1.26 | 0.0235 | 2.129 | 2.488 | 1.523 | 0.148 | 1.3599 | 2.125 | 2.66 |
| KRTAP3-3 | 684073II [symbol] | −0.0215 | −0.0255 | −0.2755 | −0.0775 | 1.433 | 0.0125 | 0.1955 | −0.2525 | 0.111 | 0.3245 | 0.1785 | 0.0716 | 0.0005 | 0.3135 | −0.0265 | −0.0955 |
| KRTHA7 | 683234II [symbol] | 0.2865 | 0.6905 | −0.3435 | −0.3975 | 0.4395 | −0.1585 | −0.1095 | −0.0065 | 0.219 | 2.659 | 0.0125 | 0.6825 | 0.1655 | −0.3725 | 0.5215 | −0.3495 |
| LAMA3 | 689785II [symbol] | 1.499 | 1.084 | −0.3805 | 0.6216 | 0.7215 | −0.1335 | 0.9305 | −0.656 | 0.8465 | 0.3365 | −0.1975 | 0.0885 | 0.9525 | 0.6925 | −0.2365 | 0.0555 |
| LAMB3 | 764691II [symbol] | 0.697 | −1.46 | −0.03 | −1.417 | −1.01 | −2.06 | −0.462 | −2.197 | −0.4865 | −0.445 | −1.138 | 1.548 | −0.683 | −2.828 | −0.61 | −1.114 |
| LASS2 | 695705II [symbol] | −0.293 | −0.616 | −2.627 | −0.637 | 0.109 | −1.763 | −1.544 | −1.616 | 0.3965 | 0.185 | 0.17 | 0.746 | 0.4615 | −1.049 | 0.073 | 0.404 |
| LAX | 682690II [symbol] | −0.3505 | −0.0435 | 0.1885 | 0.7365 | 0.4585 | 0.3695 | 0.0628 | 0 | 0.2035 | 0.3605 | 0.0195 | −0.3545 | −0.5325 | −0.4015 | −0.1145 | 0.0535 |
| LBR | 681309II [symbol] | 0.7837 | −0.8533 | −0.5383 | −0.3583 | 0.1157 | 0.2737 | 0.3717 | −0.0098 | 0.3847 | 0.0098 | −0.1263 | −0.6963 | −0.2398 | −0.4773 | −0.2943 | 0.8407 |
| LCE1B | 693796II [symbol] | −2.129 | −2.397 | −0.031 | −1.797 | 0.074 | −0.867 | 0.257 | −0.33 | −0.0265 | −0.063 | 0.393 | 0.674 | 0.638 | 0.58 | −0.697 | −2.031 |
| LDHB | 683774II [symbol] | 0.7575 | −0.6215 | −1.355 | 1.479 | −1.199 | 0.8775 | 0.7505 | −0.4535 | −1.293 | 0.2635 | −0.9065 | −1.627 | −1.825 | 0.5515 | −0.7505 | −0.2535 |
| LDOC1L | 692299II [symbol] | −0.172 | −0.499 | −0.373 | −0.506 | −0.213 | −0.36 | −1.434 | −0.4975 | −1.002 | −0.556 | 0.272 | 0.563 | 0.5835 | 0.266 | −0.683 | −0.351 |
| LETMD1 | 682701II [symbol] | 0.998 | 0.116 | −0.025 | 0.486 | 0.225 | 0.064 | −0.35 | −0.475 | −0.3085 | −0.037 | −0.389 | −0.106 | 0.742 | 0.571 | 0.025 | −0.075 |
| LFNG | 683275II [symbol] | −0.2245 | −0.2695 | 1.527 | 0.0885 | 0.7435 | 0.8935 | −0.9515 | 0.0455 | 0.567 | 2.103 | −0.2915 | 0.7175 | −0.246 | 1.438 | 1.059 | 1.227 |
| LGALS2 | 696910II [symbol] | 0.752 | −0.19 | 0.963 | 0.042 | −0.4 | 1.486 | 0.242 | 0.066 | 0.007 | 0.375 | −0.543 | −1.574 | −1.341 | −0.731 | −0.769 | 1.699 |
| LGTN | 694133II [symbol] | 0.536 | −0.359 | −0.215 | −0.287 | 0.237 | 0.535 | 0.113 | 0.108 | −0.2695 | −0.857 | −0.471 | 0.852 | 0.313 | −0.012 | −0.123 | −0.068 |
| LNX | 686766II [symbol] | −0.7075 | −0.0405 | −0.0595 | 0.4405 | 0.7895 | −0.7685 | 0.3185 | −1.413 | −1.039 | 0.0405 | 0.7425 | 0.5025 | −0.6855 | 0.5515 | −0.3495 | −0.4665 |
| LOC11247 | 687612II [symbol] | −3.285 | −2.479 | −0.747 | −2.513 | −2.515 | −2.005 | −1.177 | −1.648 | −2.91 | −2.817 | −1.63 | −2.931 | −2.764 | −2.775 | −2.231 | −2.229 |
| LOC11492 | 691804II [symbol] | −0.725 | −0.163 | −1.414 | −0.519 | −0.049 | −0.632 | 0.588 | −1.039 | −0.5605 | 0.734 | 0.885 | 1.03 | 0.153 | 0.235 | −0.16 | −0.616 |
| LOC11497 | 691267II [symbol] | 0.338 | −0.842 | 0.258 | −0.304 | −0.052 | 0.282 | 0.381 | 0.855 | −0.1985 | −0.704 | 0.017 | −0.211 | −0.0625 | −0.819 | −0.017 | 0.022 |
| LOC11621 | 686452II [symbol] | −0.9385 | 0.3175 | 1.269 | 0.1655 | −0.2745 | 0.0715 | −0.9335 | −0.4405 | −0.111 | 0.6765 | 0.1595 | 0.7705 | −0.0375 | 0.1115 | −0.4985 | −0.5935 |
| LOC11644 | 695259II [symbol] | 0.293 | 0.396 | 0.443 | 0.81 | 0.35 | 0.069 | 0.479 | 0.3305 | 0.5815 | 0.701 | 0.683 | 0.282 | 0.445 | 1.388 | 0.194 | 0.593 |
| LOC12022 | 693599II [symbol] | −1.809 | −0.633 | −0.097 | −2.039 | −1.507 | 0.145 | −2.583 | 0.0155 | −1.429 | −2.346 | −0.573 | 0.017 | −1.405 | −3.098 | −2.193 | 2.212 |
| LOC12422 | 694408II [symbol] | 0.099 | 1.935 | 2.056 | 2.667 | 3.382 | 2.895 | 1.4 | 2.6375 | 3.566 | 3.378 | 2.938 | 2.669 | 2.6575 | 3.38 | 2.731 | 3.181 |
| LOC14654 | 687013II [symbol] | −0.2615 | 0.0935 | −0.1775 | 0.2455 | 0.6165 | −0.0535 | −0.1915 | 0.0995 | 0.5425 | −0.5235 | −0.4645 | 0.9295 | 0.32 | 0.1415 | 0.1905 | −0.4615 |
| LOC1550 [symbol] | 683455II [symbol] | −0.597 | 0.227 | −1.308 | 0.469 | 0.145 | −2.572 | −2.782 | −3.099 | −0.5675 | 0.832 | −1.029 | −0.611 | −0.579 | −0.268 | −1.254 | −1.11 |
| LOC2018 [symbol] | 694158II [symbol] | 0.165 | 0.55 | 0.168 | 0.273 | 1.003 | 0.618 | −0.927 | 0.481 | −0.3575 | 0.667 | 0.021 | 1.609 | 0.0415 | 0.615 | −0.069 | 1.264 |
| LOC2539 [symbol] | 689291II [symbol] | 0.429 | −0.354 | −0.088 | 0.223 | −0.991 | −0.041 | 0.465 | −0.351 | −0.2025 | −0.057 | −0.939 | 1.04 | 1.292 | 2.944 | 0.656 | −0.438 |
| LOC3399 [symbol] | 685165II [symbol] | 0.2085 | 2.944 | −0.7945 | −0.1195 | −0.5615 | −0.5885 | 0.8815 | 1.078 | −0.0245 | −0.0015 | 0.7255 | −0.0475 | 1.972 | 0.7655 | 0.0015 | 1.489 |
| LOC34817 | 686995II [symbol] | 0.003 | −0.335 | −0.007 | 0.566 | 0.233 | 0.577 | 0.003 | −0.012 | −0.0625 | −0.003 | 0.302 | −0.351 | −0.3045 | −0.042 | 0.554 | −0.322 |
| LOC4004 [symbol] | 683489II [symbol] | −0.9845 | 0.8805 | −0.4555 | −0.0005 | 0.4075 | 2.099 | −0.2205 | −0.3755 | −0.6985 | 0.5845 | 0.0005 | 0.1005 | −0.0655 | 0.6025 | −0.3965 | 0.3965 |
| LOC4408 [symbol] | 696169II [symbol] | 0.84 | 2.259 | −0.533 | 0.09 | 1.213 | 1.163 | 1.605 | 1.7205 | 0.3925 | 1.349 | 1.188 | −0.394 | 1.4835 | 1.039 | 1.704 | 0.394 |
| LOC4938 [symbol] | 684635II [symbol] | −0.194 | 0.306 | 1.399 | −1.074 | −0.373 | −0.194 | 0.736 | 0.4195 | −0.639 | −1.284 | 0.706 | 0.286 | −0.423 | −0.504 | 0.289 | 1.376 |
| LOC51123 | 697791II [symbol] | −2.636 | −2.292 | −2.149 | −1.871 | −2.807 | −2.207 | −1.8 | −0.758 | −1.9965 | −2.172 | −2.657 | −2.514 | −2.4585 | −2.449 | −2.152 | −2.782 |
| LOC5116 [symbol] | 695670II [symbol] | 0.867 | 1.618 | −0.521 | 0.884 | 2.731 | −0.533 | −0.294 | 0.9105 | 2.4345 | 1.505 | 0.484 | 1.636 | 1.112 | 1.77 | 0.912 | 0.703 |
| LOC5132 [symbol] | 694469II [symbol] | −0.54 | −0.97 | −0.649 | −0.604 | −0.585 | −1.265 | 0.511 | 0.198 | −0.5225 | 1.628 | −0.866 | 0.386 | −0.1875 | −0.435 | −0.392 | 0.491 |
| LOC5176 [symbol] | 694904II [symbol] | 0.484 | 2.536 | 0.357 | 1.994 | 2.464 | 1.93 | 0.559 | 2.3175 | 1.0935 | −0.794 | 1.762 | 2.577 | 2.0775 | −0.11 | 1.505 | 2.557 |
| LOC8155 [symbol] | 682974II [symbol] | 0.4885 | −0.4045 | −0.3385 | 1.234 | 0.0295 | −0.6155 | −0.8135 | 0.047 | −0.374 | 1.31 | −1.14 | −0.3295 | 0.6355 | 0.2615 | 0.0285 | −0.1575 |
| LOC8156 [symbol] | 695594II [symbol] | −0.526 | −0.88 | −0.363 | −0.731 | −0.892 | −1.008 | 0.153 | −1.0305 | −0.432 | −1.921 | −0.992 | −1.1 | −0.5535 | −1.291 | −1.276 | −1.482 |
| LOC9035 [symbol] | 690384II [symbol] | 0.0595 | 0.3965 | −0.5065 | 0.2825 | −0.0555 | −1.818 | −0.8765 | −1.225 | −0.1525 | 1.16 | 1.189 | −1.797 | 0.256 | 0.0895 | 0.0555 | −0.1715 |
| LOC9070 [symbol] | 691788II [symbol] | −1.199 | −1.078 | −0.7805 | 0.9285 | 0.5375 | 0.3035 | −1.041 | 0.4225 | −0.467 | 0.3655 | −0.5805 | −0.0855 | −0.9705 | 1.564 | −1.039 | −0.0025 |
| LONP | 694535II [symbol] | −0.869 | 0.527 | 0.772 | 2.546 | 0.744 | −0.063 | −0.112 | −0.8675 | −0.335 | 0.132 | 0.464 | 0.67 | −0.634 | 0.661 | 0.457 | −0.468 |
| LRAP | 694401II [symbol] | −1.002 | 0.372 | −0.107 | −0.041 | 0.137 | −0.908 | −0.914 | −0.066 | 0.4755 | 0.665 | 0.572 | −0.087 | 0.0855 | 1.032 | −0.55 | 0.407 |
| LRBA | 689006II [symbol] | −0.7023 | −0.6023 | −1.373 | −0.2683 | 0.4187 | −0.5103 | −0.0953 | −1.134 | −0.0168 | −0.0473 | 0.8537 | 0.3127 | 0.1772 | 0.6027 | −0.2043 | −0.5243 |
| LRIG1 | 696908II [symbol] | 0.886 | 2.337 | 0.888 | 2.697 | 3.064 | 1.899 | 0.554 | 0.97 | 1.6775 | 1.991 | 0.765 | 2.987 | 2.3605 | 2.211 | 1.236 | 1.364 |
| LRP6 | 688139II [symbol] | 0.1025 | 0.3385 | −1.485 | 0.4335 | 0.0715 | 0.1635 | 0.5835 | −0.2435 | 0.1235 | −0.1155 | −0.2195 | −0.6725 | 0.287 | 0.1855 | 0.6275 | −0.3115 |
| LRP8 | 684430II [symbol] | −1.284 | −1.188 | 0.954 | −0.618 | −1.165 | 0.393 | 1.369 | 0.534 | −0.224 | 0.02 | −0.02 | −0.982 | −1.252 | −0.77 | −0.976 | 0.655 |
| LRRC17 | 689237II [symbol] | 0.5445 | −0.3675 | 0.0155 | −0.3145 | 0.8315 | −0.1165 | 0.4635 | −0.7825 | −0.3325 | 1.434 | −0.0735 | −0.0155 | 0.4305 | −0.1695 | 0.1755 | −0.2625 |
| LRRC2 | 696221II [symbol] | 0.298 | 0.999 | 0.884 | 0.676 | 1.187 | 0.142 | −0.172 | 0.0255 | −0.2035 | 1.936 | 1.996 | 1.356 | 0.578 | 0.927 | 1.511 | 1.079 |
| LSM1 | 693186II [symbol] | 0.07 | −0.56 | −1.151 | −0.414 | −0.768 | −0.16 | 0.548 | −0.8635 | −0.9355 | −0.351 | 0.56 | 0.173 | −0.29 | −0.116 | −0.457 | −0.541 |
| LSM8 | 689434II [symbol] | −0.637 | 0.009 | 0.019 | −0.067 | 2.349 | 0.019 | 0.163 | 0.0155 | −0.239 | 0.03 | 0.371 | −0.009 | 0.4055 | −0.622 | 0.238 | −0.125 |
| LTB | 695788II [symbol] | 3.772 | 2.589 | 3.654 | 2.986 | 2.826 | 4.432 | 3.828 | 4.0385 | 2.185 | 3.74 | 2.625 | 1.042 | 0.884 | 1.975 | 1.782 | 3.948 |
| LY6D | 688248II [symbol] | 0.018 | −0.095 | 2.673 | −0.673 | −0.579 | 0.088 | 1.163 | 0.431 | 1.7 | −0.51 | −0.286 | −0.309 | −0.2305 | −0.296 | −0.525 | 0.049 |
| LYN | 690653II [symbol] | −0.17 | −0.355 | 0.121 | 0.278 | −0.199 | 0.097 | 1.93 | 0.5005 | −0.876 | −0.257 | −0.01 | −0.62 | −0.2775 | 0.302 | −0.304 | 0.717 |
| MAD2L1 | 684649II [symbol] | −1.23 | −0.33 | 1.06 | −0.664 | −1.3 | −0.871 | 0.22 | −0.8125 | −1.263 | 0.463 | 0.829 | −0.476 | −0.5575 | −0.61 | −0.51 | 0.482 |
| MADP-1 | 687178II [symbol] | −0.3745 | −0.1685 | −0.4225 | 0.2345 | 0.2175 | −0.3385 | 0.0515 | −0.4245 | −0.52 | −0.0235 | 0.6505 | 0.2565 | 0.072 | −0.0775 | 0.1845 | −0.0955 |
| MAGEA12 | 696395II [symbol] | 0.116 | −0.052 | −1.329 | −0.806 | −1.112 | −1.043 | −2.383 | −0.9455 | −1.4125 | 0.389 | 1.09 | −1.657 | 1.601 | −2.154 | −0.976 | 0.76 |
| MAGEA2 | 696763II [symbol] | −3.576 | −3.296 | −3.572 | −2.955 | −3.428 | −3.201 | −2.086 | −0.4455 | −3.768 | −3.81 | −4.067 | −3.969 | −3.8185 | −3.81 | −2.954 | −4.013 |
| MAGEA3 | 689265II [symbol] | −0.1865 | −0.2265 | −1.196 | 0.3235 | −0.0525 | −0.8675 | −1.18 | −0.208 | 1.075 | −0.1085 | 0.7975 | 1.017 | 0.5875 | −0.0165 | 0.9925 | −0.3085 |
| MAGEA4 | 690918II [symbol] | 0.3225 | −0.5455 | 0.5585 | 0.1145 | −0.4495 | 0.7005 | 1.869 | 2.856 | 0.177 | −0.1335 | −0.1985 | −0.5145 | 0.2445 | −0.5135 | −0.0295 | 0.0935 |
| MAGED4 | 683069II [symbol] | 1.119 | 0.0565 | 0.1095 | −0.3765 | −0.8585 | 0.0065 | 2.07 | 0.2625 | −0.347 | 0.0395 | 0.1915 | −0.3715 | −0.2815 | −0.4235 | 1.395 | −0.3685 |
| MAGI1 | 688247II [symbol] | −0.2665 | −0.7935 | 1.526 | 3.035 | 1.055 | −0.0075 | −0.1935 | −0.579 | −1.814 | −1.407 | −1.658 | 0.2525 | −2.031 | 4.199 | −1.463 | 0.8735 |
| MAGI-3 | 692838II [symbol] | −0.298 | 1.394 | 1.319 | 0.484 | 1.041 | 1.593 | 0.507 | 1.042 | 0.114 | 1.309 | 1.38 | 1.253 | 0.753 | 1.811 | 0.924 | 0.588 |
| MAL2 | 683958II [symbol] | 0.576 | 0.382 | 1.083 | −0.115 | −0.976 | 1.623 | −0.321 | 2.446 | −2.038 | −0.767 | 0.452 | −0.359 | −1.456 | −0.61 | −1.411 | 0.09 |
| MAN2B1 | 690298II [symbol] | 0.353 | 0.348 | −0.4 | −0.403 | −0.596 | 0.182 | −0.591 | 1.023 | 0.577 | 0.12 | 0.479 | 0.025 | −0.1965 | 0.204 | 0.001 | 0.268 |
| MAPK8IP [symbol] | 693222II [symbol] | 0.166 | 0.976 | 1.948 | 2.371 | 0.872 | −0.071 | 0.209 | 2.8355 | 2.161 | 1.953 | 2.399 | 2.387 | 1.8115 | 2.798 | 1.396 | 0.913 |
| MARCO | 691594II [symbol] | −0.6075 | −0.4845 | 3.02 | −0.3655 | −0.0445 | −0.5435 | 1.151 | 1.874 | −0.06 | 0.0465 | −0.2165 | 0.0215 | −0.0215 | −0.2855 | −0.4995 | 0.6955 |
| MARLIN1 | 687609II [symbol] | 0.4645 | 0.2495 | 1.535 | 0.9545 | 0.9175 | −0.1035 | −0.1835 | 1.151 | 0.4505 | 0.3455 | −0.4705 | −1.199 | −0.5675 | 3.33 | −0.3425 | −0.1725 |
| MASS1 | 687008II [symbol] | −0.6208 | −0.0998 | −0.8038 | −0.7358 | −0.4518 | 0.9532 | −1.187 | 1.017 | 0.0998 | 0.7552 | 0.7122 | 0.1392 | 0.5222 | 1.012 | −0.2408 | 2.215 |
| MCM2 | 694711II [symbol] | −1.655 | −0.538 | −0.047 | −0.824 | −0.593 | 0.161 | −0.018 | −0.3995 | −0.493 | −0.833 | −0.058 | −0.513 | −0.6895 | −0.642 | −0.507 | −0.448 |
| MCM3 | 696856II [symbol] | −1.475 | −1.818 | 0.313 | −1.117 | −1.284 | −1.028 | −0.192 | −1.333 | −1.939 | −1.295 | −1.309 | −1.621 | −1.374 | −1.379 | −1.359 | −1.217 |
| MESP1 | 697846II [symbol] | 0.269 | 0.138 | 1.614 | 2.375 | −0.317 | 3.172 | 3.107 | 2.833 | 1.665 | 2.985 | 0.118 | −0.807 | −0.5525 | −1.483 | 0.355 | 3.999 |
| METRN | 691541II [symbol] | −0.6545 | −0.1825 | −0.1165 | 0.6205 | 0.5775 | −0.4245 | −2.108 | −1.492 | 0.925 | −1.028 | 0.1065 | 1.242 | −0.582 | 0.8595 | 0.5175 | 0.0665 |
| MFAP2 | 682204II [symbol] | 0.2885 | 0.5295 | 0.5555 | −1.257 | −1.175 | 0.3735 | 0.8325 | 1.338 | 0.511 | −1.372 | 0.9205 | −0.0145 | 0.9165 | 0.0985 | 1.272 | −0.6725 |
| MFGE8 | 695022II [symbol] | −0.026 | −0.053 | −0.005 | 2.075 | 0.243 | 0.044 | 0.656 | 0.094 | −0.1375 | −0.331 | −0.152 | −0.511 | −0.393 | −0.395 | 0.048 | −0.291 |
| MGAT4B | 687726II [symbol] | −0.0725 | −0.2345 | −0.3645 | −0.2955 | −0.1715 | −0.5145 | 0.5935 | 0.027 | −0.702 | −0.1365 | 0.5415 | 0.1175 | −0.2415 | −0.3115 | −0.2045 | 0.0625 |
| MGC1091 | 690398II [symbol] | −0.3745 | 0.0155 | 0.6135 | −0.3675 | −0.0455 | 0.4015 | 0.9535 | 0.5035 | 0.066 | −0.3935 | 0.7815 | 0.0175 | 0.198 | −0.4505 | 0.2385 | 0.5395 |
| MGC1098 [symbol] | 687879II [symbol] | 0.5945 | −1.042 | 0.1735 | −1.432 | 0.4835 | −0.1885 | 1.519 | −1.067 | −0.1735 | 1.273 | −0.9055 | 1.731 | 1.85 | −1.417 | −0.3385 | 0.2225 |
| MGC1124 [symbol] | 680903II [symbol] | −0.5255 | −0.0825 | 0.2675 | 0.3625 | 0.1285 | −0.9685 | −0.9235 | 0.197 | 0.67 | 1.269 | 0.1505 | 1.368 | 0.849 | −0.5455 | 1.159 | 1.634 |
| MGC1127 | 697307II [symbol] | −0.496 | −0.025 | 0.551 | −1.319 | −0.879 | −1.643 | −0.464 | 0.0215 | −0.2555 | −1.293 | −1.042 | 0.044 | −0.563 | −0.662 | −0.226 | −0.835 |
| MGC1312 [symbol] | 695012II [symbol] | −0.525 | −0.652 | −0.135 | −0.283 | −0.216 | 0.256 | 0.126 | 0.0655 | −0.326 | −0.861 | −0.015 | −0.392 | −0.3145 | −0.423 | −0.442 | −0.348 |
| MGC1318 [symbol] | 687992II [symbol] | 0.0795 | 0.3835 | 0.4755 | −0.2845 | −0.1605 | −0.1535 | 0.6235 | 0.156 | 0 | −0.2755 | −0.4255 | −0.7615 | −0.0395 | −0.5065 | −0.1616 | −0.5305 |
| MGC1414 | 691067II [symbol] | −1.093 | 0.756 | 0.768 | 0.502 | 0.368 | −0.211 | −0.862 | 0.562 | 0.432 | 0.733 | 0.41 | −0.742 | −0.121 | 0.03 | −0.132 | −0.132 |
| MGC1428 [symbol] | 695016II [symbol] | −0.265 | −0.498 | −0.3 | 0.361 | 0.285 | 0.032 | −0.324 | −0.114 | −0.4065 | 0.61 | 0.218 | 0.076 | 0.476 | 0.3 | 0.016 | 0.47 |
| MGC148 [symbol] | 693346II [symbol] | −0.8605 | −0.1555 | −0.0705 | −0.7685 | 0.0755 | −1.233 | 1.279 | −1.285 | −0.768 | 0.9415 | 1.44 | 1.581 | 0.6855 | 1.487 | 0.0705 | −0.8085 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| MGC1560 [symbol] | 681291II [symbol] | 0.257 | 0.512 | −0.491 | 0.091 | 1.039 | −0.211 | −0.216 | −0.5855 | −0.147 | 2.239 | −0.091 | 0.488 | 0.6585 | −0.188 | −0.31 | 0.221 |
| MGC1663 [symbol] | 695059II [symbol] | 1.14 | 3.925 | 0.2125 | −2.074 | 4.405 | −1.389 | 1.411 | 0.545 | −0.2295 | −3.418 | 0.7695 | 4.63 | 3.062 | 1.355 | 1.712 | −0.5845 |
| MGC1673 [symbol] | 684966II [symbol] | 1.045 | 0.1365 | 0.2135 | −0.4575 | 0.0495 | 0.3095 | −0.0065 | 0.024 | −0.253 | −0.1805 | 0.2595 | 0.0415 | −0.3405 | −0.1755 | −0.5435 | 0.1345 |
| MGC2053 [symbol] | 692614II [symbol] | −0.909 | −0.48 | 0.627 | −1.354 | −0.905 | −0.538 | 0.047 | −0.082 | −1.3245 | −0.805 | −1.373 | −0.922 | −0.6885 | −1.538 | −0.753 | −0.814 |
| MGC2398 [symbol] | 684508II [symbol] | 0.097 | 0.13 | 0.452 | −0.318 | −0.266 | 0 | 2.218 | 1.662 | 0.099 | −0.099 | 0.789 | 0.338 | 0.217 | −0.485 | 0.887 | −0.477 |
| MGC2610 | 685994II [symbol] | −0.3435 | 0.4225 | −0.1955 | 0.8575 | −0.1615 | −1.121 | −1.935 | −1.121 | −0.1385 | 0.6145 | 0.2245 | 0.2435 | 0.747 | 0.9705 | −0.0145 | 0.2805 |
| MGC2714 | 681884II [symbol] | −0.1825 | −0.2905 | −0.3065 | 0.0115 | −0.4325 | −0.0115 | 0.5725 | −0.53 | −0.3315 | −0.6055 | 0.9095 | 0.1835 | 0.0445 | 0.1095 | −0.3885 | 0.3625 |
| MGC2716 [symbol] | 682082II [symbol] | −0.081 | −0.391 | −0.483 | 1.405 | 1.39 | 0.104 | 0.228 | 0.4445 | 0.104 | 1.532 | 0.764 | −0.56 | −0.795 | −0.67 | 0.026 | −0.585 |
| MGC2716 [symbol] | 685085II [symbol] | 0.557 | 1.049 | −0.231 | −0.456 | −0.905 | 0.26 | 1.375 | 0.265 | 0.343 | 0.391 | 0.283 | −1.075 | 0.171 | −0.37 | 0.691 | 0.74 |
| MGC2716 [symbol] | 687819II [symbol] | −0.266 | −0.276 | −0.354 | 0.658 | 1.25 | −0.064 | 0 | 0.7755 | 0.3435 | 0.969 | 0.579 | −0.299 | −0.4365 | −0.63 | −0.2006 | −0.137 |
| MGC2716 [symbol] | 694146II [symbol] | 0.174 | 0.803 | 0.435 | 2.499 | 2.185 | 1.083 | 1.147 | 1.7465 | 1.18 | 2.011 | 0.937 | 0.554 | 0.267 | 0.444 | 0.647 | 0.742 |
| MGC2716 [symbol] | 695966II [symbol] | 0.395 | 1.135 | 0.638 | 2.685 | 3.083 | 0.966 | 0.877 | 1.5745 | 1.1065 | 1.811 | 1.504 | 0.728 | 0.426 | 0.584 | 1.556 | 0.749 |
| MGC2987 [symbol] | 692023II [symbol] | −0.1657 | −0.5087 | −0.8367 | −0.7567 | 0.1153 | −0.4137 | 0.2413 | −0.4777 | −1.156 | −0.2747 | 0.0143 | 1.05 | −0.0143 | −0.4077 | −0.4067 | 0.7033 |
| MGC3212 [symbol] | 693269II [symbol] | 0.499 | 0.014 | 0.203 | 1.231 | 1.64 | −0.524 | −0.528 | −0.4535 | 0.9035 | 0.345 | −0.39 | 0.805 | 0.993 | 2.265 | −0.346 | 0.029 |
| MGC3321 [symbol] | 692025II [symbol] | −0.123 | 0 | 0.109 | −0.16 | 0.384 | −0.241 | 0.664 | −0.1495 | 0.4555 | 0.209 | −0.34 | 0.37 | 0.4415 | −0.2331 | 0.532 | −0.53 |
| MGC3464 [symbol] | 692812II [symbol] | 2.605 | 1.318 | 2.217 | 1.852 | 0.882 | 1.56 | 3.996 | 2.722 | 1.3815 | 2.187 | 1.328 | 1.141 | 1.0165 | 1.707 | 1.078 | 2.895 |
| MGC3492 [symbol] | 691559II [symbol] | −0.653 | −0.016 | 2.606 | −0.702 | −0.495 | −0.055 | 1.167 | 0.3955 | 0.141 | −1.096 | −1.336 | −0.866 | −0.5165 | 0.32 | 0.639 | −0.307 |
| MGC4021 [symbol] | 695104II [symbol] | 0.3 | 0.422 | 0.105 | −0.014 | 3.69 | −0.238 | 0.244 | 0.1105 | 1.888 | 0.469 | 3.171 | 3.318 | 2.7775 | 0.834 | 2.51 | 0.265 |
| MGC4251 | 693489II [symbol] | 0.058 | −0.584 | 0.514 | 0.411 | 0.623 | −1.114 | −0.303 | 0.6885 | 0.452 | 0.366 | −0.718 | 0.87 | 0.099 | 0.932 | 0.916 | 0.589 |
| MGC4308 | 691305II [symbol] | −0.913 | −0.051 | 0.86 | −0.202 | −0.079 | −0.559 | 0.562 | 0.002 | −0.728 | 0.054 | 0.17 | 0.059 | −0.1315 | −0.002 | −0.526 | −0.163 |
| MGC4606 | 683786II [symbol] | −0.0045 | 0.0695 | 0.1785 | 0.1005 | 0.9025 | −0.6125 | 0.5426 | −0.805 | 0.5565 | 0.0045 | −0.4205 | 0.7335 | 0.112 | −0.6235 | 0.2975 | −0.5685 |
| MGC4659 | 684418II [symbol] | −0.1445 | 0.9665 | 0.8365 | 3.734 | 0.2505 | −0.7135 | 0.9325 | −0.531 | −0.602 | 1.771 | −0.0315 | 0.6025 | −0.712 | 1.449 | 0.0315 | 0.9245 |
| MIA | 695303II [symbol] | 0.72 | −0.764 | −2.31 | 0.06 | −2.442 | −1.67 | 2.44 | −0.381 | −1.393 | 0.01 | −1.09 | −1.28 | −2.096 | −2.17 | −0.154 | 0.81 |
| MID1 | 687747II [symbol] | −0.2685 | −0.0795 | −0.8155 | 0.6395 | −0.2845 | 0.4245 | −1.113 | −0.4115 | −0.7245 | −0.4175 | −0.4605 | −0.0085 | 0.474 | 0.5265 | −0.6055 | 0.0235 |
| MID1 | 690115II [symbol] | 0.872 | −0.373 | −1.378 | 1.485 | −1.325 | 0.665 | 1.288 | 0.4495 | −0.5805 | −0.047 | 0.101 | −0.887 | −0.782 | −0.732 | −0.784 | 1.775 |
| MID1 | 690599II [symbol] | 1.174 | −1.082 | −1.298 | −0.396 | −1.137 | 0.94 | 1.246 | 0.475 | −0.097 | 0.133 | 0.097 | −0.747 | −0.844 | −0.584 | −0.551 | 1.881 |
| MLF1IP | 684863II [symbol] | 0.0093 | −1.884 | 0.1322 | −1.654 | 0.4562 | 0.2392 | −1.474 | −0.0093 | 0.0818 | 0.5262 | −1.939 | 1.098 | −1.936 | 0.5132 | 0.4282 | −0.1188 |
| MLSTD1 | 689505II [symbol] | 0.0775 | −0.3515 | 1.417 | 0.2365 | 0.7405 | −0.6675 | 0.4725 | −0.47 | −0.6955 | −0.2785 | 0.3195 | 1.467 | −0.823 | −0.4225 | −0.6325 | 0.1905 |
| MMP1 | 697289II [symbol] | −3.971 | −2.198 | −0.174 | −2.706 | −0.837 | −1.431 | 0.992 | 0.6135 | −2.6985 | −3.413 | 0.462 | −1.558 | −2.7695 | −2.119 | −3.344 | −3.118 |
| MMP7 | 697274II [symbol] | 3.326 | 3.204 | 1.66 | 3.19 | 2.873 | 1.567 | 4.624 | 3.0655 | 2.011 | 1.832 | 4.239 | 2.344 | 2.718 | 2.72 | 3.005 | 4.092 |
| MNAT1 | 687038II [symbol] | 0.0587 | 1.33 | −1.47 | 0 | 0.03 | 0.443 | −1.32 | −0.49 | 0.79 | −0.58 | 0.093 | 1.908 | 0.95 | 0.157 | −0.26 | −0.454 |
| MRPL14 | 686347II [symbol] | −1.059 | −0.56 | 0.498 | −0.201 | −0.119 | −0.335 | 0.616 | −0.323 | −0.3785 | 0.385 | 0.453 | −0.549 | −0.905 | 0.232 | −0.422 | −0.091 |
| MRPL15 | 693549II [symbol] | −1.207 | −0.668 | −0.421 | −1.078 | −1.371 | −1.113 | −0.637 | −0.9565 | −1.4105 | −1.298 | −0.922 | −1.176 | −1.24 | −1.45 | −1.683 | −0.402 |
| MRPL27 | 697164II [symbol] | −0.313 | −0.27 | −0.015 | −0.651 | −0.46 | −0.054 | 0.152 | 0.4805 | −0.346 | 0.85 | −0.467 | −0.271 | 0.153 | −0.402 | −0.243 | 0.036 |
| MRPL34 | 688498II [symbol] | −0.118 | 0.177 | 0.168 | 0.007 | 0.144 | −0.354 | −0.088 | −0.352 | −0.878 | 1.188 | −0.407 | −0.167 | 0.3355 | 0.231 | −0.158 | −0.001 |
| MRPL43 | 683308II [symbol] | −0.3715 | −0.0395 | 0.2355 | 0.1365 | −0.2425 | −0.1445 | 0.6025 | 0.5036 | −0.6525 | 1.513 | 0.5955 | 0.2695 | −0.7635 | 0.3735 | 0.0395 | 1.071 |
| MRPL45 | 687058II [symbol] | −0.2255 | −0.6465 | 0.0865 | 0.0405 | 0.2025 | 0.7905 | −0.7185 | −0.3165 | −0.431 | −0.6825 | −0.9855 | 0.5535 | −0.0305 | −0.2535 | −0.3525 | 2.004 |
| MRPL48 | 683204II [symbol] | 0.3385 | 0.1115 | −0.0015 | −0.3435 | −0.1525 | 0.0345 | 0.5125 | 0.2015 | −0.0785 | −0.5015 | 0.1045 | −0.2595 | −0.5525 | 0.0015 | −0.6795 | 0.4035 |
| MRPL9 | 682558II [symbol] | −0.2675 | −0.3115 | −0.2085 | −0.4675 | 0.3175 | 0.0895 | 0.6845 | 0.1775 | −0.0715 | −0.7305 | −0.2175 | 0.4795 | 0.0115 | 0.4885 | −0.1375 | 0.3335 |
| MRPS14 | 691200II [symbol] | 0.2815 | 0.0165 | −0.0845 | −0.0065 | 0.0065 | −0.1735 | −0.7125 | −0.0525 | −0.755 | −0.3105 | −0.3495 | 0.4495 | 0.0345 | 0.4675 | −0.0505 | −0.1195 |
| MRPS15 | 686445II [symbol] | −0.871 | 0.163 | 0.263 | −0.033 | −0.561 | 1.745 | −0.16 | 0.187 | 0.9445 | −0.166 | −0.431 | −0.403 | −0.25 | 0.207 | −0.834 | −0.138 |
| MRPS17 | 695189II [symbol] | 2.079 | 1.79 | 2.862 | 2.928 | 1.966 | 2.633 | 2.651 | 2.609 | 1.2965 | 2.913 | 2.147 | 1.598 | 1.449 | 3.494 | 1.509 | 3.035 |
| MRPS24 | 681503II [symbol] | −0.01 | 0.018 | −0.042 | −0.198 | −0.383 | −0.31 | 0.253 | 0.307 | 0.061 | −0.282 | 0.447 | 0.01 | 0.541 | −0.309 | −0.208 | 0.042 |
| MRPS25 | 695169II [symbol] | −1.874 | −0.669 | −0.512 | −0.231 | −1.261 | −0.715 | −1.195 | −0.6955 | −0.497 | −1.471 | −1.766 | −1.399 | −0.6485 | −0.794 | −1.716 | −1.133 |
| MRPS31 | 689671II [symbol] | −0.254 | −0.461 | −0.881 | −0.303 | 0.305 | 0.299 | −0.502 | −0.1995 | 0.117 | 0.14 | 0.332 | 0.105 | 0 | 0.02 | 0.326 | 0.085 |
| MRPS6 | 692172II [symbol] | 0.153 | −0.043 | −0.219 | −0.381 | −1.59 | −0.861 | −0.263 | −0.354 | −1.0995 | −0.703 | −0.126 | 0.135 | −0.941 | −0.975 | −0.674 | −0.127 |
| MS4A1 | 690488II [symbol] | 0.631 | −0.051 | −0.099 | 0.11 | 0.399 | 1.05 | −0.025 | 0.2005 | 0.86 | 0.61 | 0.049 | −0.343 | −0.0905 | −0.079 | −0.182 | 0.402 |
| MSH2 | 686029II [symbol] | −0.964 | −0.427 | 0.51 | −0.96 | −0.455 | −0.681 | 0.035 | −0.051 | −0.462 | 0.511 | 0.308 | 0.343 | −0.4145 | −0.463 | 0.026 | −0.01 |
| MSH3 | 692736II [symbol] | −1.222 | −0.647 | −0.454 | −0.493 | 0.159 | −0.437 | −0.587 | −0.655 | −0.6655 | 0.146 | 0.291 | 0.484 | 0.0415 | 0.158 | −0.376 | 0.763 |
| MSMB | 695442II [symbol] | −0.273 | −0.02 | −0.687 | −0.749 | −0.579 | 0.613 | −0.02 | −0.189 | 0.9425 | 0.953 | 0.04 | 0.594 | 0.02 | −1.072 | 1.421 | 0.534 |
| MTA1 | 690961II [symbol] | −0.2555 | 0.0435 | 0.2445 | −0.6945 | 0.1515 | −0.0325 | −0.2025 | 0.6515 | 0.322 | −0.4735 | 0.0395 | 1.373 | 0.1585 | −0.5595 | 0.3835 | 0.0325 |
| MTA3 | 684982II [symbol] | −0.12 | 0.2 | −0.093 | −0.345 | 0.426 | −0.744 | −0.388 | −0.885 | −0.187 | 0.184 | −0.003 | 0.172 | −0.374 | −0.503 | 0.084 | −0.522 |
| MTAC2D1 | 689442II [symbol] | −1.011 | −0.165 | 0.988 | 0.83 | −0.019 | 0.007 | 0.433 | −1.356 | 1.094 | −0.588 | 0.23 | 0.77 | 0.425 | 0.087 | −0.109 | −0.072 |
| MTCH1 | 694039II [symbol] | 0.199 | −0.569 | −0.343 | −0.091 | −0.854 | −0.523 | −0.305 | −0.101 | −1.522 | −0.367 | −0.166 | −1.253 | −0.7085 | −0.313 | −0.431 | −0.292 |
| MTHFD1 | 693532II [symbol] | −2.082 | −1.151 | −0.316 | −1.157 | −0.906 | −1.032 | −1.276 | −1.898 | −2.2155 | −1.673 | −1.403 | −0.949 | −1.0225 | −1.205 | −1.19 | −1.647 |
| MTMR2 | 683517II [symbol] | 0.27 | −0.176 | −0.08 | −0.4 | 0.166 | 0.365 | 0.296 | 0.1015 | 1.507 | 0.439 | 0.114 | −1.735 | 0.3475 | −0.739 | 0.464 | 0.617 |
| MUC1 | 691634II [symbol] | −1.784 | 0.2055 | −0.0405 | −2.818 | 0.3675 | −1.869 | −2.009 | −0.3255 | 1.368 | 0.1325 | 0.2846 | 0.4605 | 0.372 | −0.7885 | 0.9325 | 0.9245 |
| MUC20 | 691484II [symbol] | −0.322 | −0.181 | −1.093 | −0.492 | 0.657 | 1.218 | 2.309 | 1.334 | 0.4865 | −0.784 | 2.102 | 0.116 | 0.1205 | −0.917 | −0.154 | 1.645 |
| MUM1 | 684755II [symbol] | 0.791 | −0.249 | 0.125 | 0.035 | 0.555 | 1.153 | 0.099 | 0.5435 | −0.1135 | −0.767 | −0.149 | 0.097 | −0.0595 | −0.543 | 0.134 | −0.035 |
| MXI1 | 692501II [symbol] | 0.865 | 0.723 | −0.986 | −0.242 | 0.407 | −0.143 | −0.627 | −0.152 | −0.093 | 0.833 | −0.401 | 0.584 | −0.4905 | 0.663 | −0.302 | 0.213 |
| MYB | 693882II [symbol] | −0.081 | 1.185 | 0.762 | 1.076 | 1.104 | 0.462 | −0.646 | 0.446 | 0.6825 | −0.173 | 1.336 | 0.323 | 1.2855 | 1.211 | 1.083 | 0.496 |
| MYBL1 | 685207II [symbol] | 0.565 | 0.239 | −0.798 | 0.305 | −0.947 | −0.535 | 0.555 | −0.7825 | −0.5285 | −0.035 | −0.145 | 0.605 | 0.288 | −0.835 | −0.975 | 0.035 |
| MYBL2 | 683074II [symbol] | −1.468 | 0.3405 | 1.189 | −0.7975 | −0.5745 | 0.1475 | 1.34 | 1.073 | −0.7155 | −1.119 | 0.4615 | −1.012 | −0.3085 | −1.334 | −0.4755 | −0.3495 |
| MYO10 | 682215II [symbol] | −0.5685 | 0.1555 | 0.9565 | −1.595 | −0.1765 | −0.0365 | 1.272 | 0.276 | −1.724 | −1.167 | 0.3695 | −0.4205 | 0.529 | −0.3435 | −1.103 | 0.4825 |
| MYO6 | 695860II [symbol] | −0.8985 | −0.2765 | −0.2625 | −0.0325 | 0.0325 | 3.092 | 2.611 | −0.2655 | 0.297 | 0.3355 | −0.4235 | −0.4916 | −0.6505 | −0.4225 | 0.7095 | 2.148 |
| MYST3 | 772896II [symbol] | 0.3697 | 0.25 | −0.758 | 0.687 | 0.856 | 0.591 | 0.754 | 0.169 | 0.531 | 0.953 | 0.806 | 1.065 | 1.021 | 0.61 | 0.654 | 0.325 |
| NALP2 | 692203II [symbol] | 0.347 | −1.31 | −1.233 | −0.659 | 0.287 | 1.579 | −0.634 | 1.306 | 0.4445 | −1.777 | −1.424 | −2.075 | 0.739 | −1.001 | −1.233 | 1.031 |
| NALP6 | 684014II [symbol] | −0.301 | 0.136 | −0.201 | 0.015 | 0.012 | 0.021 | 0.017 | 0.1835 | −0.012 | 0.08 | −0.08 | 0.093 | −0.162 | 1.116 | −0.018 | 0.36 |
| NANS | 697330II [symbol] | −1.463 | −0.096 | 1.193 | −0.293 | −0.188 | 0.05 | −0.619 | 0.211 | 0.6505 | 0.325 | 0.342 | −0.649 | −0.133 | −0.313 | −0.366 | 0.474 |
| NARF | 687421II [symbol] | −1.281 | −0.7715 | 0.2415 | −0.1505 | −0.0325 | 1.482 | 0.4135 | 0.2765 | −0.9205 | 1.176 | −0.8715 | −0.4435 | −0.4545 | 0.2175 | 0.1245 | 0.8675 |
| NAT2 | 697768II [symbol] | −1.202 | −0.81 | 0.19 | −0.889 | 0.093 | −0.693 | −0.217 | 0.7025 | −0.0645 | 0.062 | 0.241 | 0.577 | 0.371 | 0.491 | −0.427 | −0.871 |
| NAV2 | 685130II [symbol] | 1.203 | 0.9075 | −0.2675 | 0.8985 | 0.3065 | 0.7285 | −0.0095 | 1.651 | −0.042 | 1.132 | −0.5285 | 0.1335 | 0.0135 | −0.1205 | −0.2395 | −0.2005 |
| NDE1 | 690898II [symbol] | −0.1545 | 0.9605 | 0.2365 | −0.3665 | −0.0295 | 0.3985 | 0.2815 | 0.8695 | 0.25 | −0.3125 | 0.2395 | 0.9715 | −0.166 | −0.4435 | 0.5755 | 0.1135 |
| NDP52 | 684577II [symbol] | 0.4735 | 0.0145 | 0.0785 | 0.1435 | 0.7895 | −1.167 | −0.6855 | −0.228 | −0.8135 | 0.1705 | −0.1825 | 0.3205 | −0.019 | −0.3165 | −0.0205 | 0.5435 |
| NDRG2 | 686705II [symbol] | 1.112 | −0.06 | −0.5 | 0.245 | 0.527 | −0.306 | 2.469 | −0.209 | 0.7205 | −0.186 | −0.924 | −0.162 | 0.316 | 0.06 | 0.414 | 0.885 |
| NDUFA7 | 682916II [symbol] | 0.091 | −0.32 | −0.802 | 0.68 | 0.275 | −0.402 | −0.573 | −0.567 | 0.4795 | −0.08 | −0.431 | 0.071 | 0.6535 | 0.545 | −0.004 | 0.107 |
| NDUFB1 | 695310II [symbol] | −1.206 | −0.173 | 0.242 | 0.106 | 0.728 | 0.578 | −0.227 | −1.0165 | 0.606 | −0.293 | 0.125 | 0.561 | 0.311 | 0.59 | −0.093 | 0.227 |
| NDUFB10 | 681833II [symbol] | −0.379 | −0.315 | 0.035 | 0.497 | 0.543 | −0.035 | −0.247 | 0.1935 | 0.887 | 0.282 | −0.073 | 0.136 | −0.121 | 0.378 | 0.168 | 0.359 |
| NDUFB2 | 688062II [symbol] | −1.003 | −0.0175 | 0.0175 | 0.3325 | −0.3555 | −0.3775 | −0.3465 | −0.254 | 0.1715 | 0.4675 | 0.4915 | −0.0535 | 0.2095 | 0.4375 | 0.1385 | 0.0565 |
| NDUFB5 | 686352II [symbol] | −0.3245 | −0.0625 | 0.0095 | −0.0475 | −0.2565 | −0.3785 | −0.4426 | −0.3565 | −1.081 | 0.1985 | 0.0065 | 0.0125 | 0.1425 | 0.1425 | −0.8775 | 0.2905 |
| NDUFB6 | 686626II [symbol] | −0.8515 | 0.0325 | −0.4535 | 0.2955 | 0.2145 | −0.4825 | −0.6275 | −0.1425 | −0.4135 | 0.4935 | 0.9075 | −0.2815 | −0.3655 | 0.4375 | −0.3635 | 0.4126 |
| NDUFC2 | 696213II [symbol] | 1.745 | 0.027 | −0.088 | 0.044 | −0.345 | −1.205 | −0.908 | −0.88 | −1.066 | −0.032 | 0.042 | −0.445 | −0.711 | 0.435 | −0.806 | −0.323 |
| NEBL | 690781II [symbol] | −0.8585 | 0.3445 | −0.5185 | −0.4745 | 0.0065 | −0.5845 | 2.594 | −0.2275 | −0.092 | −0.0065 | 1.124 | 0.3216 | −0.4345 | 0.4475 | −0.6955 | 0.2935 |
| NEK2 | 695375II [symbol] | −1.746 | −2.55 | −1.022 | −3.036 | −2.676 | −1.102 | −1.23 | −1.719 | −2.55 | −2.455 | −1.343 | −1.583 | −1.699 | −2.918 | −1.893 | −1.234 |
| NEK6 | 695042II [symbol] | 0.153 | 0.036 | 2.067 | 0.336 | −1.498 | 0.2 | 0.984 | 1.016 | −0.796 | −1.293 | 0.559 | −0.768 | −0.7375 | −0.762 | −0.467 | −0.258 |
| NELL2 | 688574II [symbol] | −0.138 | 0.803 | −0.807 | −0.165 | 3.535 | 0.048 | −0.333 | −0.8865 | −0.1595 | 0.559 | 0 | 0.153 | 4.994 | 1.729 | 2.519 | 0.718 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.306 | −0.534 |
| NEURL | 681900II [symbol] | −0.7705 | −0.6345 | −0.4785 | 2.894 | 0.6575 | −0.2695 | 0.2065 | −0.089 | −0.406 | 0.1135 | −0.0145 | 0.0655 | 0.458 | 1.035 | −0.0495 | −0.8175 |
| NFE2L3 | 683131II [symbol] | −0.8615 | −0.4845 | −0.7095 | −1.191 | −0.0635 | −0.6815 | 0.5285 | −0.3705 | −0.8715 | −0.2315 | 0.1785 | 0.7485 | −0.3285 | −1.651 | −1.033 | −0.2415 |
| NFIA | 692198II [symbol] | 2.674 | 2.084 | 2.47 | 1.249 | 3.094 | 3.365 | 2.619 | 3.3335 | 2.174 | 1.878 | 1.75 | 3.823 | 3.433 | 2.132 | 1.76 | 3.115 |
| NFIB | 681529II [symbol] | −0.034 | 0.4 | −1.193 | 0.667 | −0.191 | −0.577 | −0.53 | −0.8565 | 3.548 | 2.178 | −0.591 | 0.702 | 0.4865 | 0.702 | 0.111 | −0.121 |
| NINJ1 | 684190II [symbol] | −0.0655 | 0.8015 | −1.153 | −0.1375 | 0.8365 | −0.9455 | −1.32 | 0.0275 | −0.637 | −0.0185 | 1.183 | −0.5565 | 0.679 | 0.6455 | 0.0185 | −0.1165 |
| NIPSNAP [symbol] | 697441II [symbol] | −0.217 | 0.116 | 0.146 | −0.037 | −0.622 | −0.618 | −0.608 | −0.259 | −0.145 | 0.013 | 0.713 | −0.559 | −0.1725 | 0.685 | −0.614 | 0.278 |
| NKX6-2 | 682635II [symbol] | 0.031 | 0.05 | 0.117 | 3.386 | −0.029 | 0.971 | −0.193 | 0.3815 | 0.452 | −0.175 | −0.273 | −0.197 | 0.2395 | −0.047 | 0.281 | −0.199 |
| NME1 | 689970II [symbol] | −0.2445 | −0.6455 | 0.4425 | −0.1405 | −0.5085 | 0.2395 | 0.3905 | −0.3225 | −0.8205 | −0.2675 | −0.4645 | 0.3625 | −0.0845 | 0.4145 | 0.0135 | 1.325 |
| NME2 | 681665II [symbol] | −0.284 | −0.317 | 0.288 | −0.363 | −0.475 | 0.023 | 0.013 | 0.046 | −1.112 | −0.441 | −0.634 | 0.492 | 0.0235 | 0.539 | −0.097 | 1.344 |
| NME3 | 682225II [symbol] | −0.2345 | 0.7685 | −0.7295 | 0.7025 | 0.8445 | −0.1445 | −0.2875 | −0.37 | 0.639 | 0.1255 | 0.0835 | 0.2415 | −0.029 | −0.0465 | 1.015 | −0.0255 |
| NMES1 | 689620II [symbol] | 0.613 | 1.249 | 2.333 | 0.257 | 1.881 | 0.769 | 2.362 | 0.8385 | 2.7125 | 1.539 | 1.126 | 1.966 | 2.21 | 0.239 | 1.725 | 1.455 |
| NMI | 690834II [symbol] | −0.435 | −0.289 | 1.007 | −0.155 | −0.113 | −0.095 | 0.515 | 1.297 | −0.298 | 0.245 | 0.315 | 0.035 | −0.3515 | −0.185 | −0.306 | 1.015 |
| NOC4 | 691497II [symbol] | −0.5865 | −0.0705 | 0.5025 | 0.0705 | −0.4805 | −0.4685 | 0.2455 | −0.1445 | −0.8705 | 0.1875 | 0.1515 | −0.2235 | −0.8175 | 0.2525 | −0.2385 | −0.1185 |
| NOLC1 | 689311II [symbol] | −0.815 | −0.267 | 0.239 | −0.059 | −0.24 | −0.157 | −0.963 | −0.6345 | −0.641 | 0.07 | 0.044 | 0.079 | −0.602 | −0.106 | −0.355 | 0.163 |
| NotFound [symbol] | 681142II | 1.096 | −0.396 | −1.051 | −0.657 | −0.9 | 0.873 | 1.176 | 0.81 | −0.451 | 0.041 | −0.041 | −0.77 | −0.919 | −0.504 | −0.162 | 1.48 |
| NotFound [symbol] | 681155II | 0.0465 | −0.8015 | −0.0695 | 0.4225 | 0.0505 | 0.0105 | −0.3125 | −0.8675 | −1.218 | −0.7805 | −0.5975 | 0.3475 | −0.3545 | 0.0205 | −1.14 | 0.6915 |
| NotFound [symbol] | 681349II | −0.0955 | −0.0955 | −0.1845 | 0.1765 | 0.0275 | −0.2025 | −0.0715 | −0.371 | −0.1575 | 0.4735 | 0.1875 | 0.2195 | −0.738 | 0.2595 | −0.2865 | 0.4255 |
| NotFound [symbol] | 681357II | −0.5475 | −0.1705 | −0.0205 | 0.0925 | 0.9225 | 0.0795 | 0.0025 | 0.8835 | 0.2195 | 0.5565 | −0.0775 | −0.4435 | −0.2145 | −0.7145 | −0.3665 | −0.2915 |
| NotFound [symbol] | 681576II | 0.5655 | 1.48 | 0.0465 | −0.4145 | 0.3685 | 0.5235 | −0.7585 | −2.016 | −0.1465 | 1.163 | 0.3415 | 0.3375 | −0.578 | 0.0395 | −0.1915 | −0.3135 |
| NotFound [symbol] | 681606II | −1.405 | −0.115 | 0.589 | 0.485 | −1.158 | 0.71 | 0.921 | −0.3215 | −0.073 | −0.911 | 0.077 | −0.876 | −0.2515 | 0.37 | −0.407 | −0.141 |
| NotFound [symbol] | 681958II | 0.0495 | −0.2255 | 1.136 | 0.3485 | −0.6065 | 0.8825 | 0.0635 | 0.4275 | −0.73 | 0.3185 | 0.4945 | −0.5285 | −1.584 | −0.0955 | −1.36 | 1.46 |
| NotFound [symbol] | 682219II | −0.06 | 1.251 | 0.326 | 0.741 | 0.078 | −0.455 | −0.306 | −0.394 | 0.079 | −0.251 | −0.025 | 0.536 | 0.1175 | 0.013 | −0.147 | 0.046 |
| NotFound [symbol] | 682298II | −0.222 | 0.238 | 0.286 | −0.078 | −0.191 | −0.148 | 0.92 | 0.2635 | −0.032 | 0.914 | 0.032 | −0.363 | 0.266 | −0.314 | −0.741 | −0.342 |
| NotFound [symbol] | 682699II | 1.869 | 1.877 | −0.296 | 2.823 | 3.423 | 3.716 | −2.012 | −0.965 | 4.197 | 4.961 | 4.366 | 2.152 | 1.7135 | 4.72 | 5.373 | 3.464 |
| NotFound [symbol] | 683354II | 0.629 | 0.571 | 0.109 | 0.242 | 0.404 | 0.396 | 1.37 | 0.9155 | 0.0995 | 0.401 | 0.367 | 0.277 | 0.5305 | 0.27 | 0.21 | 0.297 |
| NotFound [symbol] | 683540II | 1.48 | 1.085 | −1.628 | 0.0205 | 1.373 | −0.3655 | 1.1 | 0.365 | −0.4595 | 1.025 | −0.8915 | −0.7865 | −1.069 | 1.517 | 0.0775 | −0.0205 |
| NotFound [symbol] | 683552II | −0.8265 | 0.0025 | 0.7125 | 0.1035 | −0.0025 | 0.2505 | 0.3375 | 0.772 | 0.0245 | −0.1055 | 0.0845 | −0.8795 | −0.714 | −0.0525 | −0.3165 | −0.2925 |
| NotFound [symbol] | 683669II | 0.096 | 0.197 | −0.024 | 0.264 | −0.25 | 0.281 | 1.634 | 0.5935 | 0.679 | −0.267 | −0.048 | −0.232 | 0.0565 | −0.195 | −0.274 | −0.09 |
| NotFound [symbol] | 684000II | −0.422 | 1.319 | −2.299 | 0.962 | 0.889 | 0.095 | −0.133 | 0.943 | 2.369 | 1.118 | −0.755 | 0.473 | 1.495 | −2.388 | 0.327 | 0.103 |
| NotFound [symbol] | 684333II | −0.173 | 0.31 | 0.51 | 0.622 | 0.58 | 1.762 | 2.563 | 0.1995 | 0.4705 | 0.198 | 0.344 | 0.316 | 0.0865 | 0.33 | 0.28 | 1.662 |
| NotFound [symbol] | 684481II | −1.409 | 0.7585 | −0.2585 | −0.5185 | 0.0225 | −0.0655 | −0.7615 | 0.3905 | 0.489 | −0.1925 | 0.9745 | −0.1375 | −0.2265 | 0.1425 | 0.3795 | 0.4795 |
| NotFound [symbol] | 685169II | −0.408 | 0.413 | −1.127 | −0.018 | 0.457 | −0.576 | −0.992 | −0.2765 | 0.162 | 1.439 | 0.413 | 0.359 | −0.177 | 0.357 | 0.934 | 0.103 |
| NotFound [symbol] | 685265II | −0.2725 | −0.0775 | −0.5645 | 0.0095 | −0.4335 | 0.5855 | 0.2565 | 1.084 | −0.8065 | −0.2385 | −1.021 | 0.7545 | 0.096 | −0.7525 | −0.2115 | 0.0855 |
| NotFound [symbol] | 685572II | 0.4 | −0.369 | −0.765 | 0.166 | −0.535 | 0.123 | −0.281 | −0.121 | −0.916 | −0.048 | 0.161 | 0.199 | −0.6265 | 0.203 | −0.014 | 0.342 |
| NotFound [symbol] | 665625II | −0.2445 | 0.9035 | 0.1775 | −0.1565 | 0.9455 | −0.3435 | −0.3785 | −0.3345 | 0.29 | 0.9725 | −0.0195 | 0.1095 | −0.0525 | 0.0315 | 0.0195 | −0.1065 |
| NotFound [symbol] | 685756II | 1.553 | 1.604 | −1.449 | 0.102 | −0.058 | −0.451 | −1.153 | −0.804 | 0.1575 | 2.224 | −0.437 | −0.067 | −0.156 | 2.106 | 0.342 | −0.597 |
| NotFound [symbol] | 685823II | 0.468 | 0.229 | −0.292 | −0.212 | 0.271 | 0.205 | −0.79 | 0.4965 | 0.328 | −0.026 | −0.015 | 0.064 | 0.4015 | 0.21 | 0.27 | 0.106 |
| NotFound [symbol] | 686027II | 0.294 | 0 | 0.158 | −0.389 | −0.796 | 0.064 | 1.958 | 0.8085 | −0.538 | −0.28 | 0.433 | −0.52 | −0.615 | −0.2611 | −0.282 | 0.269 |
| NotFound [symbol] | 688055II | −0.503 | 1.12 | −0.285 | 0.113 | 4.505 | −0.661 | −0.642 | −1.109 | 3.2 | 0.894 | 4.162 | 3.79 | 4.099 | 1.636 | 3.304 | −0.113 |
| NotFound [symbol] | 686477II | −2.212 | −0.325 | 0.058 | −0.714 | −0.136 | −0.351 | 0.87 | −0.8025 | −0.2235 | 0.832 | 0.707 | 0.571 | 0.835 | 0.515 | 0.141 | −0.521 |
| NotFound [symbol] | 686759II | 0.476 | −0.075 | −0.575 | 1.733 | −0.331 | 0.022 | −1.013 | −0.574 | 0.1055 | 2.599 | 0.437 | 0.568 | 0.778 | 0.392 | −0.228 | 0.305 |
| NotFound [symbol] | 686948II | −0.162 | 0.016 | −0.016 | −0.824 | 0.308 | −0.477 | 0.058 | −0.381 | −0.7835 | −0.629 | −0.323 | 0.879 | −0.216 | −0.563 | −0.186 | −0.22 |
| NotFound [symbol] | 687368II | −2.236 | −0.2885 | 0.6065 | −0.5595 | 0.2305 | −0.3535 | 0.8505 | −0.8645 | −0.678 | 0.7505 | 0.4825 | 0.1925 | 0.566 | 0.1565 | 0.1425 | −0.7585 |
| NotFound [symbol] | 687761II | −0.2905 | 0.7175 | −1.433 | 0.0805 | −0.7225 | −1.148 | −1.155 | −0.1755 | −0.709 | 0.6455 | 1.131 | 0.2025 | −0.1465 | −0.3825 | 0.0385 | −0.3285 |
| NotFound [symbol] | 688033II | 0.5895 | −0.0095 | −1.225 | −0.2575 | −0.5735 | −0.5225 | −1.235 | −1.197 | −1.083 | 0.7995 | 0.3905 | 0.3945 | 0.578 | 0.1295 | −0.2475 | 0.2425 |
| NotFound [symbol] | 688163II | 0.2855 | 0.0555 | −0.3285 | 1.002 | 0.6865 | 0.7065 | 0.3835 | 0.0765 | −0.2575 | 1.966 | 0.2325 | −0.4115 | −0.339 | −0.6035 | −0.1055 | −0.1355 |
| NotFound [symbol] | 688264II | −1.389 | −0.4635 | 0.2725 | −0.2675 | 0.7155 | −0.9695 | 0.2445 | −0.183 | 0.2515 | 1.707 | 1.026 | 1.968 | 0.5535 | 0.6585 | 1.004 | 0.4525 |
| NotFound [symbol] | 688843II | −1.941 | 0.9535 | −1.017 | −0.1445 | −0.0295 | 0.0295 | 1.399 | −0.55 | −0.4375 | 1.004 | 0.5335 | 0.9125 | 0.84 | 1.206 | 0.5245 | −0.3825 |
| NotFound [symbol] | 688964II | −1.277 | −0.2695 | 0.3595 | −0.8615 | −0.1495 | −0.4675 | 0.9645 | −0.613 | −0.3765 | 1.121 | 0.8215 | 0.4975 | 0.714 | 0.4295 | −0.0745 | −0.4985 |
| NotFound [symbol] | 688988II | −0.927 | −0.236 | −0.426 | −0.008 | 0.569 | 0.586 | −0.426 | −1.14 | 0.9935 | 0.717 | −0.173 | 1.624 | −0.454 | 0.822 | 0.008 | −0.266 |
| NotFound [symbol] | 689368II | −1.151 | 0.4145 | 0.1575 | −0.2785 | −0.0035 | −0.2415 | 0.4955 | −0.1175 | 0.006 | 0.4715 | 0.0725 | 0.2255 | 0.5285 | 0.3815 | 0.0035 | −0.9315 |
| NotFound [symbol] | 689732II | −1.24 | 0.458 | −1.377 | −0.324 | 0.623 | −1.926 | 0.045 | −1.771 | −0.795 | 0.676 | 1.2 | 0.551 | 1.278 | 1.168 | −0.215 | −0.85 |
| NotFound [symbol] | 690073II | −0.9015 | 0.0295 | 1.282 | 0.7305 | −0.8285 | −0.4375 | −0.2355 | 0.0245 | −0.653 | −0.1755 | 0.0035 | −0.5035 | −1.39 | 0.8415 | −0.0035 | −0.3415 |
| NotFound [symbol] | 690533II | −2.801 | 1.18 | −0.5685 | −1.055 | 0.4915 | −0.4815 | 1.076 | 0.0715 | 0.437 | 0.7465 | −0.0715 | 0.9355 | 0.6045 | 0.3225 | 0.8505 | −0.8915 |
| NotFound [symbol] | 690571II | 0.143 | 0.021 | 0.229 | −0.012 | 0.028 | 0.031 | 0.155 | 0.5335 | −0.2625 | −0.353 | −0.499 | −0.392 | −0.169 | −0.199 | 0.009 | −0.47 |
| NotFound [symbol] | 690683II | 0.306 | −0.154 | −0.195 | −0.321 | 0.23 | 0.4 | 0.771 | 0.0745 | −0.4115 | −0.032 | −0.33 | 0.686 | 0.7015 | 0.008 | 0.678 | −0.319 |
| NotFound [symbol] | 691307II | 0.226 | 0.234 | −0.464 | 0.095 | −0.042 | 0.165 | 0.078 | −0.229 | −0.0185 | −0.188 | 0.033 | 0.19 | 0.2345 | −0.201 | −0.007 | 0.456 |
| NotFound [symbol] | 691444II | −0.4465 | −0.3825 | −0.3225 | 0.3185 | 0.2225 | 0.3345 | −0.0275 | 1.416 | 0.128 | 0.6395 | 0.4315 | −0.1315 | −1.159 | −0.3685 | 0.0275 | −0.0435 |
| NotFound [symbol] | 691485II | −1.57 | −0.3105 | 0.7365 | −0.6435 | 0.2115 | −0.1775 | 0.7655 | −0.7205 | −0.663 | 0.6985 | 0.5465 | 0.2315 | 0.451 | 0.2425 | 0.0735 | −0.5535 |
| NotFound [symbol] | 691652II | 0.865 | 0.419 | 1.293 | 1.277 | 0.715 | 1.443 | 1.326 | 1.4045 | 1.3175 | 0.011 | −0.535 | −0.027 | −0.293 | 0.918 | 0.366 | 0.859 |
| NotFound [symbol] | 691783II | −0.0295 | 0.2395 | 0.0545 | 0.0175 | 0.1915 | 0.4565 | 0.3045 | 0.659 | 0.5895 | 0.3235 | −0.0565 | −0.2885 | 0.0715 | 0.0116 | −0.1485 | −0.2345 |
| NotFound [symbol] | 692077II | −0.1665 | −0.2335 | −0.2235 | −0.1985 | 0.0185 | 0.2615 | 0.3775 | −0.105 | 0.206 | −0.3085 | −0.1855 | 0.1735 | −0.1615 | −0.1365 | 0.5646 | −0.3045 |
| NotFound [symbol] | 692188II | 0.598 | 1.073 | −0.409 | 0.031 | 1.145 | 0.923 | 0.063 | 1.3525 | 0.2315 | −0.005 | 0.324 | 1.117 | 1.312 | 0.432 | −0.358 | 0.506 |
| NotFound [symbol] | 602468II | 0.608 | −0.364 | −0.311 | −0.294 | 0.462 | −0.242 | 0.55 | −0.7815 | −0.3455 | −0.362 | −0.57 | 0.305 | −0.667 | 0.356 | 0.556 | −0.126 |
| NotFound [symbol] | 692727II | −1.151 | 0.446 | 0.734 | −0.055 | 0.203 | 0.139 | −0.189 | 0.568 | 0.11 | 0.089 | 0.579 | −0.213 | −0.5415 | −0.044 | 0.126 | 0.624 |
| NotFound [symbol] | 692860II | 0.16 | −1.291 | −2.032 | −0.229 | −0.465 | −0.792 | −1.728 | −1.7925 | −1.749 | −1.302 | −0.945 | −0.314 | −1.5115 | 0.002 | −1.223 | −0.683 |
| NotFound [symbol] | 692931II | −0.11 | −0.171 | −0.225 | −0.564 | −0.033 | 0.062 | −1.037 | −0.903 | −0.4175 | −0.524 | −1.806 | 0.997 | −1.136 | −0.2 | 0.526 | −0.762 |
| NotFound [symbol] | 693121II | 2.599 | 3.563 | 2.065 | 1.631 | 2.702 | 2.538 | 1.04 | 0.4925 | 1.452 | 3 | 2.381 | 2.397 | 1.659 | 2.112 | 1.666 | 1.779 |
| NotFound [symbol] | 693387II | −0.6 | 0.902 | 0.162 | 0.416 | 0.928 | 1.048 | −0.15 | 1.092 | 0.2765 | 0.015 | −0.424 | 0.816 | 1.528 | 1.112 | 0.735 | 0.663 |
| NotFound [symbol] | 693511II | 1.128 | 3.618 | 1.085 | 1.533 | 2.569 | 0.453 | 0.342 | 0.435 | 1.9195 | 2.994 | 2.132 | 0.92 | 3.4205 | 1.971 | 1.719 | 1.376 |
| NotFound [symbol] | 693741II | 0.191 | −0.458 | −0.449 | −0.329 | −0.651 | −0.673 | −1.06 | −1.4805 | −0.9695 | 0.405 | −0.569 | 0.297 | −0.106 | −0.157 | −0.167 | −0.357 |
| NotFound [symbol] | 694011II | 1.107 | 2.677 | 1.944 | −0.131 | 1.32 | 2.187 | 2.393 | 0.279 | 1.9355 | 0.775 | 1.785 | 4.513 | 3.8305 | 0.716 | 3.08 | 1.094 |
| NotFound [symbol] | 694287II | 1.426 | 1.702 | −0.04 | 5.785 | 3.721 | 1.345 | 0.069 | 1.096 | 3.2585 | 6.443 | 0.521 | 4.268 | 0.448 | 7.077 | 2.012 | 2.022 |
| NotFound [symbol] | 694458II | −0.4045 | 0.1705 | 0.0975 | 0.3585 | 0.1325 | −0.3095 | −0.1155 | −0.699 | 0.0015 | 0.1595 | −0.0365 | 0.1315 | −0.0245 | −0.2815 | −0.1075 | −0.1825 |
| NotFound [symbol] | 605199II | 0.877 | 0.508 | −0.595 | −0.026 | −1.285 | −0.024 | 1.73 | 1.345 | −0.7665 | −1.101 | −0.872 | −1.367 | −1.096 | 0.791 | 0.024 | 0.239 |
| NotFound [symbol] | 695260II | −0.56 | −0.248 | −0.25 | −0.446 | −0.854 | −0.924 | 0.255 | −0.66 | −1.215 | 1.009 | −0.24 | 0.021 | −0.423 | −0.309 | −0.12 | −0.551 |
| NotFound [symbol] | 695667II | −0.976 | 1.602 | 0.765 | 2.411 | 3.059 | 1.492 | 1.236 | 1.32 | 0.8165 | 0.631 | 1.833 | 0.742 | 1.6915 | 1.541 | 2.007 | 1.232 |
| NotFound [symbol] | 686120II | −0.066 | −0.726 | 0.382 | −0.352 | −0.056 | 0.495 | 1.331 | 0.0795 | −0.384 | −0.475 | −0.195 | −0.293 | −0.144 | −0.18 | 0.071 | −0.484 |
| NotFound [symbol] | 696388II | −3.384 | 0.208 | −2.458 | −1.658 | −1.168 | −1.605 | 0.406 | −1.6265 | −1.34 | 0.068 | −0.559 | −0.166 | −0.0545 | 0.405 | −0.66 | −1.842 |
| NotFound [symbol] | 696612II | −0.569 | 0.103 | −0.335 | 0.012 | −1.326 | 0.403 | −1.04 | −1.098 | −0.6835 | 2.237 | −0.344 | 1.859 | 0.6695 | 0.963 | 1.248 | 0.627 |
| NotFound [symbol] | 698792II | −0.86 | 0.5 | 0.081 | 1.723 | 1.351 | 0.809 | 0.425 | −0.1255 | −0.271 | 0.636 | 1.273 | 0.937 | 0.095 | 0.445 | 1.246 | 0.042 |
| NotFound [symbol] | 697127II | −0.3925 | 0.1295 | 0.3635 | −0.0455 | −0.4855 | −0.0255 | 0.4835 | 0.291 | −0.6255 | −0.1475 | 0.1615 | −0.4676 | −0.209 | −0.1265 | 0.0255 | −0.3505 |
| NotFound [symbol] | 697197II | 0.011 | −0.266 | 0.228 | 0.246 | 0.364 | 0.397 | 0.542 | 0.5665 | 0.3515 | −0.124 | 0.191 | −0.096 | 0.369 | −0.13 | −0.361 | 0.175 |
| NotFound [symbol] | 697297II | −1.791 | −0.001 | 0.405 | −0.405 | 0.337 | −0.088 | 0.72 | −0.843 | −0.3645 | 0.744 | 0.435 | 0.171 | 0.7605 | 0.454 | 0.335 | −0.871 |
| NotFound [symbol] | 697403II | −0.832 | −0.627 | 0.008 | −0.817 | −0.173 | −0.892 | −0.108 | −0.332 | −0.889 | −0.92 | −0.761 | −0.422 | −0.543 | −0.601 | −0.967 | −0.167 |
| NPDC1 | 682819II [symbol] | −0.2955 | −0.0945 | 0.1685 | 1.237 | 0.3685 | −0.3575 | −1.327 | −0.0165 | 1.213 | 1.053 | −0.0395 | −0.3095 | −0.5385 | 1.615 | 0.0165 | 0.2385 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| NPEPPS | 687914II [symbol] | 0.497 | −1.11 | −0.406 | 0.29 | 0.154 | −1.039 | −1.148 | −1.577 | −1.024 | 0.377 | −0.645 | 0.795 | 0.093 | 0.137 | 0.167 | −0.018 |
| NPY | 683068II [symbol] | 0.894 | 0.395 | 0.17 | −0.991 | 0.161 | −1.847 | −2.51 | −1.727 | −1.114 | 1.236 | 1.595 | 2.883 | 0.5535 | −0.697 | 0.138 | 0.371 |
| NCO3A2 | 697754II [symbol] | 1.238 | 1.162 | 0.395 | 0.917 | 2.02 | 0.269 | 0.658 | 0.7015 | 1.3615 | 1.252 | 1.099 | 1.757 | 0.9645 | 2.339 | 0.999 | 1.722 |
| NRAS | 695502II [symbol] | 0.1255 | −0.3085 | −0.5165 | −0.1335 | −0.2445 | 0.5435 | 0.4975 | 0.4735 | 0.385 | −0.5045 | −0.7945 | −0.2985 | 0.0275 | −0.8435 | 0.4315 | 0.1075 |
| NRIP1 | 684742II [symbol] | 0.0623 | 0.2713 | −0.7397 | −0.7697 | 1.648 | −0.3717 | −1.217 | −0.9572 | −0.3347 | 0.1143 | 0.8933 | 2.613 | −0.0623 | −0.2207 | 0.3983 | −0.1147 |
| NTN4 | 686487II [symbol] | 1.472 | 0.2985 | −2.333 | 1.629 | 1.503 | −1.026 | 1.318 | −1.403 | −0.2985 | −0.4425 | 0.8355 | 1.543 | 1.467 | −0.4455 | −0.4845 | 1.035 |
| NUCB2 | 681359II [symbol] | −1.004 | −0.2345 | −0.6905 | 0.4855 | 1.738 | 0.5655 | 0.7425 | −0.1515 | 2.368 | 1.989 | 0.0295 | −0.0595 | −1.911 | 2.284 | −0.4035 | −0.6195 |
| NUDT5 | 695608II [symbol] | −1.303 | −0.261 | 0.836 | −0.87 | −0.595 | −0.84 | 0.629 | −0.343 | −1.075 | −1.187 | −0.385 | −1.196 | −1.8045 | −0.759 | −0.813 | −0.644 |
| NUDT6 | 686517II [symbol] | 0.204 | 0.348 | −0.763 | −0.575 | 0.262 | −1.505 | −0.611 | −1.033 | −0.3645 | 0.545 | 0.369 | 1.206 | 0.2725 | 0.456 | −0.139 | 0.283 |
| OCIAD2 | 695560II [symbol] | 0.352 | 0.988 | 0.851 | 0.297 | 1.283 | 0.697 | 0.805 | 1.0855 | 0.4175 | 1.131 | 1.008 | 1.031 | 0.069 | 1.057 | 0.207 | 1.032 |
| OFD1 | 683518II [symbol] | 0.125 | 0.524 | −1.264 | 0.178 | 0.557 | 0.424 | −0.766 | −0.202 | 0.5005 | −0.47 | −0.919 | −0.747 | −0.037 | −0.637 | 0.867 | −0.48 |
| OGFRL1 | 689994II [symbol] | −0.306 | −0.44 | 1.008 | 0.336 | 0.001 | −0.81 | 2.811 | −0.1925 | 0.7015 | 0.25 | 0.006 | 0.325 | −0.3735 | −0.053 | −0.793 | 0.622 |
| OPLAH | 694256II [symbol] | −0.104 | 1.893 | −0.593 | 1.108 | 0.471 | 0.623 | 0.956 | 1.982 | 1.2465 | −1.025 | 1.723 | 0.684 | 0.5755 | 0.433 | 0.862 | 1.567 |
| ORMDL3 | 663021II [symbol] | 0.219 | 0.061 | −0.092 | −0.264 | −0.205 | 1.112 | −0.336 | 1.933 | 0.2475 | 0.196 | −0.581 | −0.396 | −0.1545 | 0.364 | 0.175 | 1.487 |
| OSBPL3 | 691049II [symbol] | −0.4015 | 0.3605 | 0.3565 | −0.1455 | 0.2965 | 1.772 | 1.137 | 0.153 | −0.5045 | −0.4235 | 0.1295 | −0.3515 | −0.8755 | 0.4745 | 0.1505 | 0.0535 |
| OSR2 | 681734II [symbol] | 1.383 | 1.103 | −1.065 | −1.368 | 0.3458 | 0.2368 | −0.5172 | 1.472 | 0.0163 | −0.0163 | 2.285 | −0.4992 | −0.3722 | −0.5352 | 1.826 | 0.5778 |
| OSTF1 | 684940II [symbol] | −1.086 | −0.021 | 1.255 | −0.395 | −0.598 | −0.369 | −0.117 | −0.199 | −0.783 | 0.627 | 0.525 | −0.617 | −0.768 | 0.126 | −0.475 | −0.003 |
| P2RX1 | 681440II [symbol] | 0.736 | 0.11 | −0.212 | −0.188 | 0.143 | −0.001 | 0.001 | 0.5155 | −0.388 | 0.469 | 0.161 | −0.315 | −0.2735 | −0.421 | −0.197 | 0.135 |
| P2RY2 | 696219II [symbol] | −0.114 | −1.541 | −1.429 | −1.842 | −1.227 | −0.769 | −1.261 | −1.678 | −1.89 | −0.749 | −0.871 | −0.423 | −1.173 | −0.773 | −1.26 | −0.91 |
| PACAP | 697667II [symbol] | −1.928 | −2.171 | −2.505 | −0.227 | −1.097 | −1.26 | −2.161 | −1.64 | −2.5845 | −0.972 | −1.559 | −2.663 | −2.8895 | −1.127 | −1.85 | −1.844 |
| PADI2 | 688464II [symbol] | 0.0195 | −0.1635 | 2.483 | −0.4905 | −0.2315 | 1.056 | 1.508 | −0.111 | −0.1205 | −0.1745 | 0.0215 | 1.377 | −0.0175 | −0.1685 | −0.0255 | 1.606 |
| PAPD5 | 688131II [symbol] | −0.4735 | −0.0495 | −0.1615 | 0.1205 | −0.5685 | −0.1685 | −0.4945 | 0.1055 | −0.217 | 0.5995 | 0.1235 | 1.07 | −0.643 | 0.4605 | −0.3755 | −0.2405 |
| PAPSS2 | 688187II [symbol] | −1.099 | 1.766 | 0.3125 | −1.272 | −0.9325 | 0.3115 | −0.7405 | 1.909 | −1.177 | −1.38 | 1.232 | 1.809 | −0.9965 | 0.3105 | 0.2445 | 1.876 |
| PARD6A | 692612II [symbol] | 0.132 | 0.266 | 0.563 | 1.254 | 0.261 | 0.563 | −0.313 | 0.458 | 0.808 | 0.691 | 0.335 | 0.415 | 0.089 | 1.547 | 0.433 | −0.109 |
| PARP1 | 683432II [symbol] | −0.1465 | −0.2255 | 0.3905 | −0.4075 | 0.4145 | 0.9005 | 0.1265 | −0.1455 | −0.4785 | −0.4975 | −0.6115 | 0.5695 | 0.8465 | −0.7945 | 0.0595 | −0.1535 |
| PARP9 | 695188II [symbol] | 1.209 | 1.649 | 0.621 | 0.885 | 1.524 | 1.039 | 0.738 | 0.263 | 0.217 | 0.813 | 0.303 | 1.638 | 1.464 | 1.77 | 1.255 | 1.048 |
| PAXIP1L | 684118II [symbol] | 1.302 | −0.2738 | −1.694 | 2.246 | −0.2728 | 0.5042 | 0.5612 | 2.544 | −0.1653 | −0.2838 | 0.1652 | −0.8928 | −1.855 | −0.8258 | −0.2928 | 0.8742 |
| PBX3 | 692245II [symbol] | −0.135 | −0.467 | −0.224 | −0.695 | 0.467 | −1.035 | −0.769 | 0.9965 | −0.5165 | 0.782 | 0.733 | 0.88 | −0.32 | 3.083 | −0.379 | 0.526 |
| PCDHB1 [symbol] | 681640II [symbol] | 0.6195 | −0.1625 | 0.9865 | 0.0016 | −0.7615 | −0.4825 | 0.2835 | −0.4985 | −0.261 | 0.2505 | 0.7155 | 0.1625 | −0.895 | 0.3325 | −0.1105 | 0.3565 |
| PCDHB16 | 697521II [symbol] | −1.12 | −2.28 | −0.085 | −0.282 | −2.344 | −1.84 | 0.292 | −0.5145 | −1.3455 | −2.202 | 0.465 | −2.34 | 0.0655 | −2.463 | 0.117 | −1.725 |
| PCDHB2 | 684692II [symbol] | 0.075 | −0.358 | 2.714 | 0.0604 | 0.902 | −1.053 | −0.924 | −0.5875 | 1.35 | 1.208 | 1.517 | 1.05 | −0.763 | −0.877 | −0.365 | 0.011 |
| PCM1 | 685108II [symbol] | −0.3375 | −0.8715 | −1.142 | 0.2235 | 1.012 | 0.0095 | −0.3525 | −1.225 | 0.184 | 0.3895 | 0.9595 | 1.578 | 0.7955 | 0.5385 | 0.4225 | −0.1295 |
| PCNA | 683820II [symbol] | −1.046 | −1.55 | 0.71 | −0.117 | 0.05 | 0.711 | 0.965 | 0.6905 | −0.01 | 0.055 | −0.101 | 0.729 | 0.0095 | −1.15 | −0.12 | 0.717 |
| PDCD1 | 687556II [symbol] | −0.07 | −0.005 | 0.333 | 0.366 | −0.161 | 0.629 | 0.2 | 0.618 | 0.2045 | 0.127 | 0.07 | −0.331 | −0.116 | −0.194 | −0.169 | 0.492 |
| PDCD10 | 694505II [symbol] | −1.281 | −0.767 | −0.444 | −1.013 | −1.012 | −1.402 | −0.827 | −0.715 | −1.741 | 0.111 | 0.457 | −0.259 | −1.086 | −0.803 | −1.537 | −0.471 |
| PDCD4 | 692083II [symbol] | −0.361 | −0.302 | −0.327 | −0.151 | −0.109 | −0.008 | −0.618 | −0.805 | −0.758 | −0.442 | −0.294 | −0.113 | 0.1985 | −0.125 | −0.317 | −0.587 |
| PDCD5 | 682521II [symbol] | −1.229 | −0.057 | −0.143 | −0.133 | −0.116 | −0.553 | 0.914 | 0.951 | −0.227 | 0.057 | 0.495 | −0.219 | −0.4755 | −0.24 | −0.474 | −0.304 |
| PDCD8 | 697583II [symbol] | −0.884 | −0.06 | 0.332 | 0.286 | −0.124 | −0.027 | −1.049 | −0.4615 | −0.51 | 1.005 | −0.28 | −0.684 | −0.063 | 0.156 | −0.433 | 0.169 |
| PDE4D1P | 689720II [symbol] | −1.385 | −1.544 | 1.305 | 1.122 | −0.9375 | 0.8665 | −0.8265 | 0.8225 | 1.809 | 0.2345 | −0.0975 | −0.7485 | −0.4175 | −1.187 | −0.6665 | 0.0265 |
| PDHA1 | 684251II [symbol] | −0.343 | −0.613 | −0.524 | 0.187 | 0.66 | 1.034 | −0.441 | 0.295 | 0.093 | 0.074 | −0.289 | −0.074 | −0.721 | 1.123 | −0.216 | 0.139 |
| PECI | 689878II [symbol] | 0.121 | −0.18 | −0.015 | 0.421 | 0.337 | −0.804 | −2.499 | −1.242 | −0.062 | 0.139 | −0.328 | −0.192 | −0.1 | 0.351 | −0.725 | 0.253 |
| PEG10 | 687554II [symbol] | −2.137 | 0.9535 | −1.031 | −2.38 | −2.784 | 0.4375 | −2.085 | −2.412 | −2.762 | −0.0395 | 1.311 | 1.881 | 1.378 | −2.3 | 1.327 | 0.0395 |
| PER2 | 688213II [symbol] | 0.9782 | 0.6042 | −1.297 | 0.7712 | 0.6872 | −0.3658 | −1.165 | −1.33 | −0.0743 | 0.8262 | −0.1428 | 0.4532 | −0.0633 | 0.0633 | 0.6112 | 0.5052 |
| PERP | 683121II [symbol] | 0.158 | −0.336 | 0.858 | −1.415 | −1.355 | −0.917 | 0.717 | −0.7765 | −1.151 | 0.102 | −0.931 | 0.094 | −0.396 | −4.017 | 0.215 | 0.399 |
| PEX1 | 686946II [symbol] | −0.5915 | −0.0395 | −0.0495 | 0.1225 | −0.0735 | 0.2505 | −1.032 | −0.8375 | −0.5385 | 0.3635 | 0.5545 | 0.7115 | 0.111 | 0.0945 | 0.0835 | 0.0435 |
| PFAAP5 | 683100II [symbol] | 0.41 | −0.128 | −0.692 | 0.012 | 1.16 | −0.121 | −0.035 | 0.004 | 0.9195 | 0.335 | 0.282 | 0.226 | 0.311 | −0.16 | 0.765 | 0.009 |
| PFDN2 | 687458II [symbol] | −0.643 | −0.156 | 0.462 | −0.499 | 0.179 | −0.143 | 0.182 | 0.2125 | −0.1315 | −0.728 | −0.182 | −0.227 | −0.3485 | −0.124 | −0.163 | −0.179 |
| PGM1 | 681276II [symbol] | −0.7235 | −0.7675 | 0.4895 | −0.2345 | 0.7845 | −0.1855 | 0.3335 | −0.295 | 0.1805 | 0.0055 | 0.1335 | 0.5465 | 0.604 | 0.8515 | −0.0925 | −0.6445 |
| PGR1 | 689407II [symbol] | −0.047 | 0.073 | −0.517 | 0.625 | 0.07 | −0.854 | −1.283 | −0.3085 | −0.149 | 0.396 | 0.252 | −0.394 | −0.785 | 0.556 | −0.143 | 0.09 |
| PHAX | 692593II [symbol] | −1.498 | −0.753 | −0.914 | −0.883 | −1.041 | −0.443 | −0.7 | −0.2 | −0.327 | −0.66 | 0.315 | −0.505 | −0.459 | −0.737 | −0.742 | −0.153 |
| PHGDH | 682626II [symbol] | 0.226 | 0.453 | 1.547 | −0.706 | −0.545 | 0.442 | 2.233 | 0.2155 | −0.9885 | −0.871 | −1.569 | −1.292 | 0.521 | −1.34 | −0.438 | 0.483 |
| PHYH | 689499II [symbol] | −0.622 | 0.34 | 0.709 | −0.294 | 0.664 | −0.608 | 0.868 | −0.3685 | −0.562 | −0.205 | −0.253 | 0.545 | −0.4895 | 0.326 | −1.148 | −0.48 |
| PIB5PA | 691558II [symbol] | 0.328 | 0.507 | −0.575 | 1.765 | 0.538 | −0.376 | −1.99 | 0.0955 | 0.524 | −1.666 | −0.161 | 0.321 | 1.232 | 0.146 | −0.846 | 0.005 |
| PIGT | 688446II [symbol] | −0.3845 | 0.8675 | −0.5875 | 1.268 | 0.4145 | −0.5155 | −0.2005 | −0.3485 | 1.071 | 0.5095 | 1.073 | −0.0875 | 0.4295 | 1.337 | 0.0875 | −0.3506 |
| PIM2 | 697074II [symbol] | 0.425 | −0.155 | −0.36 | −0.587 | 1.509 | −0.969 | 0.318 | −0.7425 | 0.27 | 0.725 | 0.422 | 0.566 | 0.4505 | 0.214 | 0.121 | −0.68 |
| PISD | 696650II [symbol] | 0.117 | 0.987 | 0.577 | 2.153 | 0.717 | −0.045 | −0.282 | 0.1565 | −0.311 | −0.052 | −0.372 | 0 | −0.272 | 0.055 | −0.339 | 0.027 |
| PITPNC1 | 684234II [symbol] | −0.068 | 0.133 | 0.395 | 0.074 | 0.006 | 1.384 | 0.392 | 3.785 | −0.4795 | −0.226 | −0.672 | −0.458 | −0.225 | −0.805 | −0.105 | 0.97 |
| PKIB | 681591II [symbol] | −1.142 | 0.199 | −0.199 | 2.474 | 2.932 | −2.328 | −1.065 | −2.107 | −0.545 | 2.572 | −0.332 | 1.414 | 0.6255 | 1.822 | 0.946 | −1.005 |
| PKMYT1 | 686037II [symbol] | −1.439 | −0.3605 | 0.8145 | −0.6036 | −1.321 | 0.3835 | 0.2165 | −0.0155 | −0.621 | −0.5675 | 0.2865 | −0.4455 | −0.2685 | −1.032 | 0.0155 | 0.2755 |
| PLA2G4B | 695710II [symbol] | −0.2725 | 0.8575 | 1.593 | −0.1425 | 0.5405 | 0.6465 | 0.3325 | 0.1815 | −0.738 | −1.052 | −0.3365 | −0.7135 | −0.523 | −1.046 | 0.4445 | 0.1735 |
| PLAC8 | 692243II [symbol] | −1.477 | −3.604 | −1.816 | −2.656 | −2.873 | −1.56 | −2.589 | −2.525 | −2.868 | −2.112 | −3.626 | −3.239 | −3.474 | 4.546 | −2.911 | −0.859 |
| PLCB1 | 681242II [symbol] | −0.688 | −0.022 | −3.194 | −0.762 | −2.321 | −1.818 | −1.333 | −0.8765 | −1.5405 | −1.687 | 0.121 | −2.951 | −2.9205 | −1.683 | −1.247 | −1.527 |
| PLCXD1 | 686574II [symbol] | −1.647 | −0.532 | −0.927 | 2.014 | 1.503 | 0.107 | −0.107 | 0.7545 | −0.46 | 1.211 | 1.067 | −0.813 | −1.717 | −1.478 | −0.247 | −0.371 |
| PLEK2 | 686964II [symbol] | −0.7975 | 0.4375 | 0.9505 | −0.8275 | 0.7465 | 0.2375 | 0.0625 | 0.145 | 0.167 | 0.1165 | 0.0446 | 0.3845 | 0.0295 | −0.2295 | −0.0325 | −0.4255 |
| PLOD | 690311II [symbol] | 0.1675 | 0.3525 | 0.9735 | −0.1115 | −0.2505 | 0.0445 | 1.206 | 0.653 | 0.0445 | −1.092 | −0.0875 | −0.4685 | −0.154 | 0.3005 | 0.3315 | −0.4325 |
| PNAS-4 | 695276II [symbol] | −0.756 | −0.714 | −0.099 | −1.087 | 0.537 | −0.412 | 0.332 | 0.229 | −0.648 | −0.806 | −0.131 | 0.031 | −0.3925 | −0.543 | −0.345 | 0.253 |
| PNLIPRP [symbol] | 692823II [symbol] | 0.111 | −0.255 | −1.103 | 1.909 | 0.623 | −0.647 | −0.763 | −1.182 | −0.256 | 0.097 | 0.671 | 1.015 | −0.1425 | 0.659 | −0.24 | 0.326 |
| PNMA3 | 695191II [symbol] | 0.2465 | −0.0125 | −0.4145 | −0.1765 | 1.498 | −0.5085 | −0.0025 | 0.4365 | 0.9665 | 1.538 | 0.5615 | 0.0025 | 0.1855 | 3.659 | −0.0995 | 0.7805 |
| POLB | 688699II [symbol] | −0.28 | −0.544 | 1.447 | 0.349 | −0.082 | 1.075 | −0.168 | −0.2455 | −0.8245 | 0.754 | −0.097 | −0.739 | −0.817 | 0.01 | 0.025 | 0.449 |
| POLR2F | 695452II [symbol] | −1.355 | −0.522 | −0.234 | −0.488 | −0.309 | −0.765 | −0.792 | −0.6465 | −0.467 | −1.197 | −1.085 | −0.783 | −0.9595 | −0.248 | −0.868 | −1.082 |
| POLR2H | 692429II [symbol] | −0.0485 | 0.4615 | −0.7275 | −0.4585 | 0.1355 | 0.8895 | 1.037 | 0.169 | −0.0975 | −0.1845 | −0.7315 | −1.259 | 0.52 | 0.4365 | −0.6505 | 1.338 |
| POMC | 653917II [symbol] | 0.0735 | −0.3435 | −0.2295 | 0.0035 | −0.7815 | 0.5645 | 1.159 | 0.3045 | 0.255 | −0.0995 | −0.1115 | −0.2055 | −0.282 | −0.2635 | 0.1045 | 0.1755 |
| POP4 | 687322II [symbol] | −0.111 | −0.318 | −0.559 | 0.092 | −0.32 | −0.26 | −0.001 | 0.8676 | −0.739 | 0.312 | 0.523 | 0.42 | −0.124 | −0.012 | −0.196 | 0.337 |
| POP7 | 688981II [symbol] | −0.3435 | 0.0275 | 0.2795 | 0.0345 | −0.3215 | −0.2705 | −0.4195 | −0.0275 | −0.0335 | −0.1455 | 0.1675 | −0.2595 | 0.0305 | −0.2005 | −0.3335 | −0.2355 |
| PP1057 | 694694II [symbol] | 1.884 | 2.61 | 1.661 | 1.017 | 1.434 | 2.556 | 1.226 | 3.303 | 2.314 | 1.286 | 2.541 | 2.012 | 1.8855 | 1.872 | 2.47 | 1.578 |
| PPFIA1 | 684385II [symbol] | 0.2145 | 0.4335 | −0.2225 | −0.2085 | −0.2105 | −0.5695 | 0.2845 | −0.0215 | −0.678 | −0.0175 | −0.0365 | −0.2425 | −0.31 | 0.0795 | −0.5775 | 0.0485 |
| PPFIBP2 | 688244II [symbol] | −0.138 | −0.05 | 0.211 | −0.783 | −0.059 | 0.823 | −0.783 | 0.1265 | −0.6615 | 0.771 | −0.072 | 0.353 | 0.198 | −0.655 | 0.02 | 0.88 |
| PPGB | 696506II [symbol] | 0.371 | 0.504 | 0.141 | −0.217 | −0.309 | 0.14 | 0.45 | 0.706 | −0.069 | −0.318 | 0.169 | −0.076 | 0.324 | 0.639 | 0.061 | 0.327 |
| PPHLN1 | 681157II [symbol] | −0.1455 | −0.1105 | −0.4725 | −0.3855 | 0.1545 | −0.2265 | 0.5705 | −0.324 | −0.3975 | 0.3015 | 0.9905 | 0.2625 | 0.0435 | 0.1255 | 0.4595 | 0.1745 |
| PPIF | 685607II [symbol] | −0.588 | 0.271 | 0.147 | −0.578 | −0.612 | 0.277 | −0.406 | −0.566 | 0.04 | 1.653 | −0.15 | −0.641 | 0.6295 | −1.017 | 0.519 | −0.021 |
| PPIL1 | 693349II [symbol] | −1.961 | −1.685 | −0.754 | −1.28 | −2.071 | −1.266 | −1.19 | −1.268 | −2.2485 | −1.352 | −1.076 | −1.743 | −1.848 | −1.107 | −1.657 | −0.914 |
| PPOX | 686128II [symbol] | 0.096 | 0.126 | −0.31 | −0.433 | 0.468 | −0.169 | −0.164 | 0.364 | 0.8105 | −0.058 | −0.144 | 0.623 | 0.602 | −0.077 | 0.341 | 0 |
| PPP1R16 [symbol] | 684680II [symbol] | −0.219 | 0.392 | 0.496 | 0.175 | −0.022 | −0.778 | −0.63 | −0.089 | 0.5595 | −0.21 | −0.084 | 0.187 | −0.442 | 0.052 | 0.008 | −0.008 |
| PPP1R3C | 688073II [symbol] | 1.011 | 1.408 | −0.5835 | 0.0435 | 1.87 | −0.3695 | −1.005 | 0.2255 | 0.6505 | 0.4745 | 1.197 | −0.4395 | 2.301 | −0.0435 | 1.91 | −0.1375 |
| PPP2R2D | 689931II [symbol] | −0.2765 | −0.0015 | 0.4055 | −0.0565 | 0.1865 | −0.0105 | 0.1475 | −0.1315 | −0.28 | 0.1065 | 0.4705 | 0.0085 | −0.688 | −0.3035 | −0.0755 | 0.3425 |
| PPP2R5A | 686848II [symbol] | −1.299 | 0.2155 | 0.7975 | −0.7235 | 0.0355 | −0.0235 | −0.1175 | 0.037 | 0.0495 | 0.4775 | 0.5315 | 0.6855 | 0.414 | 0.0235 | −0.0485 | 0.8435 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| PRAME | 690640II [symbol] | −0.6265 | 3.257 | 0.6065 | −0.1755 | −0.2995 | 0.7105 | −0.2295 | 2.129 | 0.181 | −0.1915 | −0.4635 | −0.3165 | −0.971 | 0.1095 | 0.9265 | 1.383 |
| PRC1 | 685404II [symbol] | 0.0055 | 0.3335 | 0.7235 | −0.0945 | −0.3985 | −0.2255 | 0.7665 | 0.3355 | −0.336 | −0.3975 | 0.5695 | −0.9285 | −0.171 | −0.7725 | −0.2635 | 0.1945 |
| PRIM2A | 687823II [symbol] | −0.512 | −0.476 | 1.135 | −0.332 | 0.124 | 0.548 | 0.846 | −0.213 | −0.236 | −0.072 | −0.133 | 0.05 | −0.1245 | −0.031 | −0.039 | 0.553 |
| PRKACB | 696518II [symbol] | 0.472 | −0.03 | −0.373 | 3.271 | −0.285 | −0.016 | −0.357 | −0.4785 | 1.3505 | 0.671 | −0.337 | −0.425 | −0.1695 | 2.204 | −0.375 | 0.115 |
| PRKCB1 | 694132II [symbol] | −1.604 | −1.919 | −1.615 | −1.383 | −0.963 | −1.112 | −1.921 | −1.2795 | −1.7105 | −1.606 | −2.71 | −2.748 | −2.2115 | −2.381 | −2.305 | −0.271 |
| PRKCI | 686943II [symbol] | 0.3132 | 0.1682 | −0.1158 | −0.2808 | 0.5302 | 0.4802 | 0.3822 | −0.0013 | −1.24 | −0.1658 | 0.4832 | 0.0013 | 0.3722 | −0.2888 | 0.4132 | −0.2938 |
| PRKRIR | 684358II [symbol] | 1.554 | 0.053 | 0.109 | 0.466 | −0.116 | 0.217 | 0.081 | −0.3795 | −0.5485 | −0.052 | 0.065 | −0.166 | −0.3455 | −0.114 | −0.358 | 0.354 |
| PRKWNK [symbol] | 683385II [symbol] | −2.111 | −1.39 | 0.228 | −1.32 | −1.804 | −0.555 | −0.347 | −1.196 | −0.7995 | −1.413 | −1.255 | −1.205 | −1.538 | −1.678 | −1.152 | −1.399 |
| PRKX | 686136II [symbol] | 0.169 | 0.46 | −0.695 | −0.698 | −0.286 | −1.024 | −1.306 | −0.6575 | −0.2705 | 0.277 | 0.282 | 0.244 | 0.2055 | 0.542 | 0.013 | −0.283 |
| PRNP | 692320II [symbol] | −0.273 | −1.642 | −2.509 | −1.32 | −3.023 | −2.955 | −1.21 | −2.293 | −3.3455 | −1.262 | −1.143 | −1.681 | −2.588 | −1.761 | −1.245 | −0.81 |
| PROL5 | 684331II [symbol] | −0.321 | 0.021 | −0.231 | 0.26 | 0.217 | 0.102 | −0.1 | 0.4445 | 0.5625 | 0.204 | −0.192 | −0.394 | −0.167 | −0.211 | −0.353 | −0.348 |
| PRPF18 | 685943II [symbol] | −0.044 | −0.055 | 0.321 | −0.388 | −0.209 | 0.098 | 1.081 | −0.142 | −0.701 | 0.172 | 1.061 | 0.735 | −0.3655 | 0.011 | −0.36 | 0.432 |
| PRPSAP [symbol] | 681061II [symbol] | −0.2575 | −0.3185 | −0.1965 | 0.2655 | −0.0695 | −0.8055 | 1.129 | −0.7935 | −1.047 | 1.157 | −0.0095 | 0.3305 | −0.12 | −0.4445 | 0.0255 | −0.2495 |
| PRSS12 | 692634II [symbol] | −0.877 | −1.496 | −1.49 | −1.141 | −1.132 | −1.524 | −0.808 | −1.701 | −1.0935 | −1.582 | −1.797 | −2.094 | −1.283 | −2.166 | −1.387 | −1.229 |
| PRSS16 | 695121II [symbol] | 1.157 | −0.603 | 0.262 | −0.125 | 1.621 | 1.281 | −0.08 | 0.511 | 1.342 | 1.38 | −0.902 | 1.239 | 0.405 | 0.806 | 0.09 | 1.341 |
| PSD3 | 688328II [symbol] | −1.33 | −0.54 | −1.229 | 0.108 | 2.901 | −0.163 | 0.237 | 0.926 | 2.441 | 0.221 | 1.145 | 1.411 | 2.726 | −1.099 | 1.403 | −0.654 |
| PSMA3 | 682239II [symbol] | −0.9385 | −0.3085 | −0.0145 | −0.2475 | −0.2545 | −0.3095 | −0.1945 | −0.495 | −0.6975 | 0.0815 | 0.6375 | 0.1395 | −0.4145 | 0.2235 | −0.4185 | 0.0505 |
| PSMA7 | 694665II [symbol] | −1.193 | 0.074 | 0.265 | −0.549 | −0.882 | −0.777 | −0.021 | 0.31 | −1.0275 | −0.116 | 0.234 | −1.137 | −0.544 | −0.475 | −0.928 | −0.217 |
| PSMC5 | 687471II [symbol] | −0.4135 | −0.0925 | 0.0655 | −0.0055 | 0.1755 | 1.518 | 0.4545 | 0.885 | −0.421 | 0.8285 | −0.5795 | −0.1335 | −0.3955 | 0.3005 | 0.8875 | 0.6315 |
| PSMC6 | 693728II [symbol] | −1.466 | −1.174 | −1.316 | −0.838 | −0.675 | −1.32 | −1.477 | −1.6335 | −1.511 | −0.487 | −0.11 | −0.341 | −0.8745 | −0.981 | −1.111 | −0.922 |
| PSMD12 | 697390II [symbol] | −0.244 | −1.419 | −0.172 | −0.717 | −0.649 | 1.09 | 0.086 | −0.862 | −0.994 | 1.588 | −0.324 | 0.155 | −0.6945 | −0.241 | −0.417 | 1.608 |
| PSMD12 | 697530II [symbol] | −2.12 | −1.821 | −0.776 | −1.808 | −1.145 | −0.415 | −0.148 | −1.487 | −1.75 | 0.21 | −1.519 | −1.237 | −1.6465 | −1.549 | −1.848 | −0.194 |
| PSMD3 | 681750II [symbol] | 0.324 | −0.02 | 0.228 | −0.184 | −0.2 | 0.848 | 0.983 | 2.153 | 0.591 | 0.089 | −0.265 | −0.685 | −0.0535 | −0.207 | −0.274 | 0.553 |
| PSPH | 685810II [symbol] | −0.152 | 0.163 | −0.383 | 0.145 | −0.012 | −0.523 | −0.363 | −1.268 | −0.323 | 1.396 | 0.664 | 0.54 | 0.012 | 0.334 | −0.123 | 0.402 |
| PTD012 | 684256II [symbol] | 0.2245 | −0.8455 | −0.5465 | 0.2775 | 0.3405 | 1.463 | −0.2865 | −0.44 | −0.4965 | −0.0445 | −0.2945 | 0.7815 | −0.3425 | 0.4845 | −0.0575 | 0.7725 |
| PTGER3 | 685654II [symbol] | −0.61 | 0.748 | −0.419 | −0.098 | 0.053 | −0.224 | −0.174 | 0.8835 | 0.0385 | −0.249 | 1.404 | 0.922 | 0.0235 | −0.128 | 0 | 0.567 |
| PTHLH | 683001II [symbol] | −0.604 | 2.454 | −1.538 | 0.026 | 0.878 | −1.23 | 0.375 | 0.184 | 0.1245 | 1.472 | 0.211 | 2.758 | −1.689 | 3.135 | −0.222 | −0.353 |
| PTK6 | 690829II [symbol] | −0.469 | 0.928 | −0.224 | −0.062 | 0.154 | −0.109 | 0.008 | 0.842 | 0.721 | −0.008 | 1.676 | 0.713 | 0.3645 | 0.522 | 0.036 | 0.901 |
| PTPN4 | 693676II [symbol] | −0.552 | 0.149 | −0.393 | 0.077 | 0.027 | −0.006 | −0.173 | 0.456 | −0.4065 | 0.537 | 0.511 | 0.086 | −0.0405 | −0.254 | 0.395 | 0.868 |
| PTPN7 | 687885II [symbol] | 0.6685 | 0.0415 | 0.5665 | 0.1315 | 0.3195 | −0.3605 | 0.2405 | 0.0735 | −0.083 | −0.1165 | −0.3955 | −0.4605 | −0.407 | −0.2815 | −0.0435 | 0.1495 |
| PTPRF | 684441II [symbol] | −0.743 | 0.608 | 1.039 | −0.456 | −0.2 | 1.527 | 0.611 | −0.1455 | −0.451 | 0.174 | 0.272 | 0.014 | −0.437 | −0.014 | −0.104 | −0.263 |
| PTPRN2 | 685456II [symbol] | 1.492 | −0.1305 | −0.2355 | 2.493 | −0.0605 | 1.736 | −0.5245 | 0.0215 | 0.5865 | 2.512 | −0.7545 | −0.4715 | 1.529 | 3.702 | 0.0845 | 0.9735 |
| PTPRT | 694229II [symbol] | 0.597 | 1.973 | 0.234 | 2.241 | 1.935 | 0.069 | −0.004 | 0.1935 | 1.911 | 2.303 | 0.448 | 0.334 | 2.5505 | 3.359 | 1.077 | 0.31 |
| PTS | 691288II [symbol] | −1.382 | −0.66 | −0.189 | 0.228 | −0.644 | −0.218 | 0.77 | −0.075 | −0.2385 | 0.047 | 0.703 | −0.182 | −0.7385 | 0.123 | −0.343 | 0.64 |
| PTTG1 | 687187II [symbol] | −0.27 | 0.273 | 0.576 | −0.077 | −0.514 | 0.517 | 1.093 | 0.8145 | −0.3445 | −0.542 | 0.641 | −0.779 | −0.1595 | −0.465 | −0.152 | 0.139 |
| PURB | 697166II [symbol] | −0.456 | −1.013 | −1.1 | −1.961 | −1.61 | −1.377 | −0.662 | −1.1395 | −1.741 | −1.16 | −0.608 | −0.89 | −1.0025 | −1.477 | −1.328 | −0.92 |
| PXMP2 | 694802II [symbol] | −2.098 | −0.369 | −0.012 | −0.293 | −1.08 | −0.758 | −0.483 | −0.6015 | −1.4705 | −1.01 | −0.134 | −0.403 | −0.8775 | −0.297 | −0.941 | −0.926 |
| PYCARD | 696817II [symbol] | −0.221 | −0.165 | 0.11 | −0.676 | 0.017 | 0.477 | 0.421 | 0.3345 | 0.076 | −0.442 | −0.325 | 0.815 | 0.0205 | −0.541 | −0.02 | 0.372 |
| PYCR1 | 683284II [symbol] | −0.6145 | −0.4875 | 0.3975 | −0.4045 | −0.7965 | 0.6655 | 1.089 | 0.3785 | −0.0405 | −0.3655 | −0.2035 | −0.0205 | −0.3025 | 0.4095 | 0.0805 | −0.2485 |
| QDPR | 695556II [symbol] | 0.224 | 0.559 | −1.393 | 0.69 | −1.016 | −1.714 | −1.621 | −1.844 | 0.0665 | 0.038 | 0.283 | 1.279 | 0.1 | 3.568 | −1.119 | −0.239 |
| QPRT | 681328II [symbol] | 0.069 | −0.345 | 0.277 | −0.296 | −0.229 | 0.516 | 1.156 | −0.043 | −0.7125 | 0.146 | −0.471 | −0.135 | −0.279 | −0.399 | 0.198 | −0.134 |
| QSCN6 | 691861II [symbol] | −0.143 | −0.114 | −0.665 | −0.301 | 0.632 | −0.681 | 0.594 | −0.418 | 2 | −1.132 | −0.366 | −0.734 | 0.2485 | 1.144 | 0.383 | 0.099 |
| RAB11FIP | 697363II [symbol] | −0.99 | −0.388 | 0.822 | −1.965 | −2.173 | −0.547 | −0.428 | 0.516 | −1.9285 | −2.084 | −1.304 | −1.484 | −2.047 | 0.005 | −0.964 | 0.517 |
| RAB11FIP | 692380II [symbol] | 3.03 | 2.394 | 2.692 | 1.808 | 3.868 | 3.675 | 3.002 | 0.517 | 1.4485 | 0.943 | −0.374 | 1.819 | −0.338 | −1.066 | 1.551 | 2.509 |
| RAB13 | 681521II [symbol] | 0.0483 | −0.0508 | −0.2077 | −0.0198 | 0.4973 | 0.4563 | 0.5493 | 0.2183 | 0.0958 | −0.6197 | −0.1947 | 0.8033 | 0.0068 | 0.2803 | 0.1183 | −0.2247 |
| RAB27A | 689787II [symbol] | −1.397 | −0.9415 | 0.9105 | −0.0305 | −0.5905 | 0.6885 | 2.503 | 0.0065 | 1.363 | −0.1425 | 0.7555 | −0.5015 | −0.596 | −0.0065 | −0.0275 | 0.7495 |
| RAB30 | 690906II [symbol] | 0.158 | −0.193 | −0.112 | −0.507 | 1.111 | −0.383 | −0.34 | −0.1705 | −0.002 | 0.019 | 0.037 | 0.15 | 0.246 | 0.36 | 0.095 | −0.267 |
| RAB34 | 687074II [symbol] | 0.508 | 1.581 | −0.427 | −0.976 | 0.696 | 0.057 | −0.134 | 0.278 | −0.2545 | 0.035 | −0.08 | 0.432 | 0.633 | −0.887 | 0.968 | −0.269 |
| RAB35 | 689469II [symbol] | −0.503 | −0.713 | 1.045 | −0.007 | −0.575 | 0.208 | 0.837 | 0.328 | −0.1955 | 0.178 | 0.17 | −0.287 | −0.638 | −0.407 | 0.124 | 0.303 |
| RAB39B | 696999II [symbol] | −1.509 | −0.667 | −1.077 | 0.908 | −0.641 | −1.311 | −1.171 | −1.28 | −1.611 | 2.957 | −1.454 | −0.973 | −0.782 | 0.831 | −0.287 | −0.527 |
| RAB3A | 686794II [symbol] | 0.2025 | 0.1165 | 0.1375 | −0.2835 | −0.0435 | 0.2275 | 0.0455 | −0.2395 | −0.0405 | 0.9285 | −0.2205 | 0.0405 | 0.047 | 0.1275 | 0.1645 | −0.3535 |
| RAB3IP | 690420II [symbol] | −0.8535 | −1.048 | −0.2355 | 0.4765 | −0.2365 | −0.0075 | 0.2645 | −0.3195 | 0.132 | 0.5085 | 0.7795 | 0.3555 | 0.579 | 0.5215 | −0.6135 | 0.6255 |
| RABEP1 | 687547II [symbol] | −1.185 | −0.9745 | −0.5065 | 0.9985 | 0.9865 | −0.7475 | −1.166 | 0.945 | 1.326 | −0.3865 | −0.6755 | 1.843 | 0.2965 | 2.383 | −0.3535 | −0.1335 |
| RABGAP1 | 694298II [symbol] | 0.32 | 0.191 | 0.586 | 0.439 | 0.349 | 0.971 | 1.331 | 0.9275 | 0.281 | 0.606 | 0.627 | 0.179 | −0.0055 | 0.522 | 0.682 | 1.136 |
| RACGAP1 | 690792II [symbol] | −3.388 | −2.284 | −2.675 | −1.534 | −2.327 | −2.137 | −1.91 | −2.703 | −2.6175 | −3.008 | −2.85 | −2.33 | −2.544 | −0.354 | −2.499 | −2.579 |
| RAD17 | 691216II [symbol] | 0.167 | −0.219 | −0.446 | −0.008 | 0.271 | −0.582 | −0.619 | −0.306 | −0.616 | 0.776 | 0.468 | 0.6 | 0.3005 | 0.439 | −0.195 | 0.487 |
| RAD51 | 692122II [symbol] | −2.761 | −2.111 | −0.193 | −2.765 | −3.108 | −1.794 | −1.743 | −0.754 | −2.6025 | −2.184 | −1.161 | −2.421 | −1.745 | −3.066 | −2.013 | −1.148 |
| RAD51AP | 687081II [symbol] | 0.818 | −0.287 | 0.658 | −0.587 | −0.799 | −0.377 | 0.115 | −0.9135 | −0.534 | −0.122 | 0.715 | 0 | 0.054 | 1.025 | −0.014 | −0.249 |
| RAD52 | 693390II [symbol] | 0.0665 | 0.8515 | −0.8275 | −0.9245 | 0.3985 | 0.7415 | 0.0075 | 0.127 | −1.838 | −1.097 | 0.3795 | 1.029 | −1.818 | −2.056 | 0.0595 | 0.0315 |
| RAD54L | 690991II [symbol] | −1.174 | 0.138 | 1.1 | 0.006 | −1.206 | 0.765 | 1.237 | 0.457 | −0.88 | −0.626 | 0.418 | −0.807 | −0.0485 | −1.186 | −0.414 | −0.637 |
| RAMP | 696334II [symbol] | −1.63 | −2.265 | −0.658 | −1.87 | −2.043 | −0.08 | −1.06 | −1.905 | −1.9475 | −1.65 | −1.47 | −1.46 | −1.431 | −2.56 | −2.03 | −1.25 |
| RANBP1 | 698056II [symbol] | −2.319 | −1.893 | −0.444 | −1.176 | −2.283 | −1.205 | −1.399 | −0.236 | −2.375 | −2.308 | −1.983 | −2.152 | −2.0675 | −1.619 | −2.242 | −1.839 |
| RARRES [symbol] | 696860II [symbol] | 1.209 | 1.619 | 1.426 | 1.723 | 0.786 | 1.66 | 6.376 | 2.941 | 0.8015 | 1.044 | 2.232 | 1.904 | 0.841 | 0.965 | 0.884 | 4.268 |
| RBBP7 | 695677II [symbol] | −0.66 | −0.194 | −0.478 | −0.393 | −0.133 | −0.847 | −0.37 | −1.2115 | −0.859 | 1.284 | 0.136 | −0.422 | 0.5245 | −0.131 | −0.65 | −1.109 |
| RBBP8 | 690099II [symbol] | −0.482 | 0.206 | −0.93 | 0.434 | 0.512 | −0.412 | −0.845 | −1.288 | −1.233 | 0.086 | 1.31 | 0.467 | 0.4045 | 0.477 | −0.781 | 0.629 |
| RBM8A | 681239II [symbol] | −0.6735 | −0.2595 | 0.3925 | −0.1575 | −0.0305 | −0.1165 | 0.2505 | 0.096 | 0.233 | −0.1585 | −0.0385 | 0.0655 | −0.2085 | −0.3915 | 0.2325 | 0.0405 |
| RBX1 | 685240II [symbol] | −0.957 | −0.125 | 0.146 | −0.038 | 0.067 | −0.073 | −0.217 | 0.044 | −0.46 | −0.449 | 0.162 | −0.056 | −0.1545 | −0.057 | −0.389 | −0.015 |
| RDHE2 | 695028II [symbol] | 1.781 | 0.338 | 0.695 | 0.702 | 0.588 | 1.157 | 1.202 | 1.027 | 0.6405 | 0.799 | 0.407 | 0.704 | 0.348 | 0.417 | 1.181 | 1.861 |
| REA | 691904II [symbol] | 0.386 | −0.008 | 0.171 | −0.048 | −0.009 | 0.006 | −0.28 | −0.421 | −0.2705 | 0.327 | 0.531 | −0.012 | 0.811 | 0.371 | 0.222 | −0.333 |
| REC14 | 685901II [symbol] | −0.7385 | 0.3455 | −0.3835 | 0.2655 | 0.3105 | −0.7215 | −0.4405 | −0.147 | −0.0005 | −0.1185 | 0.0435 | −0.2215 | −0.6055 | 0.5695 | −0.3735 | 0.1665 |
| REG1A | 695196II [symbol] | 0.184 | 0.168 | 0.361 | 0.296 | −0.068 | 0.114 | 0.297 | 0.395 | 0.242 | 0.21 | 0.209 | 0.257 | 0.157 | 0.161 | −0.04 | 0.24 |
| RELB | 684812II [symbol] | −0.058 | 0.182 | −0.174 | −0.437 | −0.19 | 0.396 | −0.132 | 1.278 | 0.452 | 0.021 | 0.675 | 0.148 | 0.1445 | −0.152 | −0.307 | 0.394 |
| REPIN1 | 683066II [symbol] | −0.0665 | 0.1965 | −0.4285 | 0.1265 | −0.0015 | −0.4355 | −0.5895 | −0.7705 | −0.453 | 0.0585 | 0.6545 | 0.0565 | 0.2225 | 0.0015 | 0.2565 | 0.0935 |
| REPS2 | 687793II [symbol] | 0.6362 | −0.3108 | 2.308 | −0.3968 | −0.5268 | 0.8482 | 1.074 | −0.1888 | −0.1183 | −0.3018 | −0.8208 | 0.1232 | −1.14 | −0.9665 | −0.4468 | −0.2258 |
| RERG | 687590II [symbol] | −0.4055 | 0.6855 | −0.6445 | 0.0145 | 0.3205 | −0.6025 | 0.0045 | −0.941 | 0.085 | 2.529 | 0.2045 | −0.2785 | 0.486 | 0.8305 | 0.1005 | −0.7375 |
| RFC4 | 684079II [symbol] | −0.3525 | 0.1185 | 0.8055 | −0.5475 | −0.2985 | 0.5995 | 0.6605 | 0.1625 | −0.8835 | −0.4325 | 0.3005 | 0.0645 | 0.2005 | −0.5185 | −0.3035 | −0.2685 |
| RGS10 | 695917II [symbol] | 0.088 | 0.803 | 0.637 | 1.076 | 0.69 | 1.217 | 0.913 | 0.6545 | 0.9435 | 0.249 | 0.607 | 0.758 | 0.333 | 1.229 | 0.426 | 1.859 |
| RHBDL2 | 684395II [symbol] | −0.173 | −0.052 | 1.113 | −1.205 | 0.037 | 0.259 | 1.346 | −0.087 | −1.034 | −0.489 | 0.444 | −0.14 | 0.0985 | −0.446 | −0.203 | 0.237 |
| RIBC2 | 695320II [symbol] | −1.037 | 0.546 | 1.79 | −0.269 | −0.114 | 1.549 | 0.297 | 1.425 | 0.5765 | −0.768 | 0.683 | 0.364 | 0.015 | −0.684 | −0.651 | −0.363 |
| RIC-8 | 690624II [symbol] | −0.636 | −0.918 | −0.416 | −0.634 | −1.367 | −1.091 | −0.672 | −0.25 | −1.54 | −0.05 | −0.322 | −0.499 | −1.412 | −0.462 | −1.494 | −0.06 |
| RIT1 | 693292II [symbol] | −0.936 | −0.702 | −0.366 | −0.597 | −0.112 | −0.429 | −0.368 | 0.2395 | −0.4535 | −1.021 | 0.413 | −0.255 | −0.793 | −0.983 | −0.749 | 0.038 |
| RLN2 | 688741II [symbol] | 0.361 | −0.306 | 0.133 | 0.316 | 3.043 | −0.38 | −0.705 | 0.141 | 2.609 | 1.899 | −0.347 | 1.853 | 1.3765 | 0.058 | 0.052 | 1.234 |
| RNF10 | 696512II [symbol] | 0.407 | 0.54 | 0.253 | 0.164 | 0.022 | 0.326 | 0.857 | 0.127 | 0.103 | 0.279 | 0.855 | −0.041 | 0.4785 | 0.361 | 0.478 | 0.484 |
| RNF135 | 691324II [symbol] | 0.142 | 0.24 | 0.037 | 0.016 | −0.854 | 0.185 | 2.019 | −0.5285 | −1.195 | −0.57 | −0.686 | −0.455 | −1.628 | −0.05 | −0.258 | −0.256 |
| RNF7 | 691075II [symbol] | −0.4712 | 0.2697 | 0.4928 | 0.4457 | −0.5483 | −0.4762 | 0.1088 | −0.0058 | −0.4237 | 0.4768 | 0.4037 | −0.1292 | −0.1302 | −0.0943 | −0.3103 | −0.3662 |
| ROPN1 | 692734II [symbol] | 0.403 | −1.118 | −0.739 | −0.491 | −1.159 | −0.671 | 1.695 | −0.856 | 0.1745 | −1.19 | −1.407 | −0.683 | −1.276 | −0.902 | −0.856 | 0.4784 |
| ABAT | 682378II [symbol] | −1.035 | 0.17 | −2.664 | 0.566 | 3.38 | −1.951 | −1.821 | −2.557 | 1.761 | 1.056 | 1.109 | 2.634 | 2.026 | 1.035 | 1.308 | −0.534 |
| RPA3 | 684912II [symbol] | −0.767 | −0.032 | 0.024 | 4.783 | 0.07 | 0.202 | 0.047 | −0.2505 | 0.231 | −0.664 | −0.677 | −0.055 | −0.2825 | 2.535 | −0.106 | −0.205 |
| RPESP | 690228II [symbol] | 0.456 | −0.377 | 1.743 | −0.928 | 0.419 | −1.572 | −0.623 | 0.5485 | −0.1195 | −0.475 | 0.171 | 1.685 | 0.0575 | 0.108 | 0.57 | 0.406 |
| RPL11 | 684149II [symbol] | 0.3 | 0.684 | 0.455 | 0.834 | 0.726 | 0.442 | 0.407 | 0.583 | 0.338 | 0.415 | 0.301 | 0.309 | 0.4875 | 0.358 | 0.072 | 0.479 |
| RPL13 | 690460II [symbol] | 0.9885 | 0.1765 | −0.0095 | 0.7695 | 0.0095 | 0.6975 | −0.6235 | −0.1695 | −0.8565 | −0.3685 | −0.2335 | −0.1805 | −0.6755 | 0.8465 | −0.0445 | −0.2085 |
| RPL13A | 697951II [symbol] | −0.118 | 0.718 | −0.77 | 0.615 | 0.075 | 0.367 | −0.541 | −0.365 | −0.5125 | −0.266 | −0.17 | −0.491 | 0.2745 | 0.161 | 0.147 | −0.154 |
| RPL15 | 682985II [symbol] | −0.5097 | 0.1333 | 1.159 | −0.1597 | −0.4857 | −0.0398 | 0.4203 | −0.1027 | 0.0008 | −0.1597 | 0.3803 | −0.4397 | −0.0813 | −0.6397 | −0.3307 | 0.1513 |
| RPL18A | 683895II [symbol] | 0.8285 | −0.1265 | 0.2935 | 0.3505 | −0.4935 | 0.5145 | −0.3775 | −0.3485 | −1.102 | −0.2015 | −0.5625 | −0.1775 | 0.409 | 0.0195 | 0.1915 | −0.1125 |
| RPL19 | 688555II [symbol] | −0.449 | −0.64 | −0.121 | 0.029 | 0.015 | 0.218 | −0.881 | 2.148 | 0.519 | −0.123 | −0.621 | 0.346 | 0.023 | 0.172 | −0.284 | 1.82 |
| RPL21 | 692099II [symbol] | −0.4005 | −0.5085 | 1.234 | −0.2065 | −0.1635 | 0.0065 | 1.2 | −0.688 | −0.8215 | 0.4035 | −0.2675 | 0.0995 | 0.112 | −0.7245 | 0.1265 | −0.1115 |
| RPL23A | 681568II [symbol] | 0.251 | 0.697 | −0.627 | 0.194 | −0.166 | 0.657 | −0.938 | −0.2225 | −0.503 | 0.222 | −0.224 | 0.904 | 0.0405 | 0.255 | −0.154 | −0.238 |
| RPL23A | 692544II [symbol] | −0.779 | −0.377 | 0.086 | −0.627 | −0.338 | −0.493 | 0.014 | −0.409 | −0.166 | −0.512 | −0.525 | −0.826 | −0.2135 | −0.631 | −0.807 | −0.892 |
| RPL26L1 | 685938II [symbol] | −0.519 | −0.214 | 1.196 | 0 | −0.257 | 0.495 | 0.146 | −0.5225 | −0.5045 | 0.5 | 0.438 | −0.077 | −1.158 | 0 | 0.016 | 0.041 |
| RPL29 | 690810II [symbol] | 0.614 | 0.338 | −0.304 | 0.242 | −0.041 | 0.598 | −0.336 | −0.468 | −0.2905 | −0.054 | −0.605 | 0.28 | 0.8345 | 0.492 | −0.044 | 0.041 |
| RPL36 | 687324II [symbol] | −0.404 | −0.475 | 0.024 | 0.343 | 0.034 | 0.578 | −0.493 | 0.1175 | 0.7685 | −0.831 | −0.821 | −0.005 | 0.259 | 0.129 | −0.056 | −0.245 |
| RPL36AL | 693407II [symbol] | −3.028 | 1.096 | −0.169 | −3.159 | −3.871 | −3.749 | −3.81 | −3.647 | −2.6825 | −1.544 | −2.462 | 1.985 | −3.089 | −3.563 | −1.577 | −0.601 |
| RPS16 | 687942II [symbol] | −0.0745 | −0.5235 | −0.8975 | −0.1105 | −0.4755 | 0.4105 | −0.1125 | 1.196 | −1.196 | −0.2325 | 0.2715 | 0.2905 | 0.1335 | −0.0175 | −0.2755 | 0.2705 |
| RPS4Y1 | 683806II [symbol] | −0.968 | 0.719 | 0.213 | −0.467 | −0.477 | −0.34 | 0.128 | −0.0685 | 0.0115 | −0.442 | 0.429 | 0.009 | 0.5315 | −0.377 | 0.026 | −0.455 |
| RRAGA | 688744II [symbol] | 0.216 | 0.29 | −0.579 | 0.57 | 0.25 | −0.461 | −0.692 | −0.1615 | −0.3375 | 0.223 | −0.077 | −0.221 | −0.0475 | 0.741 | 0.018 | 0.219 |
| RRAGD | 691801II [symbol] | 0.1837 | −0.7903 | 0.1387 | −0.3323 | −0.6833 | −0.1523 | 1.75 | 0.3602 | 0.3347 | −0.2463 | 0.3667 | 0.3977 | −0.0788 | −0.3513 | −0.3623 | 0.0928 |
| RRM1 | 684258II [symbol] | −0.4575 | 0.4515 | 0.9905 | 0.1585 | 0.1625 | −0.3575 | −0.9885 | −0.2695 | −0.702 | −0.1225 | 0.0305 | 0.1695 | −0.114 | −0.3515 | −0.4485 | −0.1575 |
| RRM2 | 694262II [symbol] | −4.534 | −2.777 | −1.292 | −3.358 | −3.758 | −2.752 | −1.934 | −1.6445 | −2.726 | −3.309 | −1.915 | −3.594 | −3.087 | −3.954 | −3.384 | −2.106 |
| RSAFD1 | 687475II [symbol] | 0.8625 | −0.1825 | −0.1345 | −0.0745 | 0.1025 | 0.2045 | −0.1975 | −0.4385 | −0.436 | 0.0105 | 0.3645 | 0.3935 | 0.351 | −0.1195 | −0.0105 | 0.0505 |
| RSNL2 | 692893II [symbol] | 1.468 | 0.513 | −1.74 | 0.293 | −0.03 | −0.572 | −0.583 | −1.699 | −1.519 | −0.374 | 0.12 | 0.536 | −1.3835 | −1.463 | −0.602 | 0.083 |
| RSU1 | 682633II [symbol] | 0.2425 | 0.0215 | 0.0645 | −0.8215 | −0.5775 | 0.4825 | 1.018 | 0.336 | −0.3915 | −0.7165 | 0.7115 | −0.2025 | −0.2.985 | −0.3635 | −0.2455 | 0.0025 |
| RTN4IP1 | 696006II [symbol] | −0.92 | −0.831 | −0.297 | −1.363 | −1.413 | −0.94 | 0.033 | 0.116 | −1.4865 | −1.476 | −0.865 | −1.199 | −1.6085 | −0.284 | −1.34 | −0.198 |
| S100A11 | 688967II [symbol] | −0.003 | 0.003 | 0.281 | −0.719 | −0.088 | 0.216 | 1.175 | 0.704 | −0.4455 | −1.249 | −0.04 | 0.078 | 0.2385 | −1.103 | 0.152 | 0.467 |
| S100A14 | 689448II [symbol] | −2.183 | −0.3325 | 1.092 | 0.4335 | 0.6415 | 1.622 | −1.356 | −0.636 | −2.074 | −0.1185 | 0.8545 | 1.325 | 3.23 | −0.1695 | −0.2895 | 0.6775 |
| S100A16 | 682683II [symbol] | 0.122 | 0.265 | 0.71 | −0.61 | −0.119 | 1.11 | −0.745 | −0.5295 | −0.5535 | −0.401 | 1.255 | 0.841 | −1.326 | 0.268 | −0.049 | 0.523 |
| S100A9 | 681875II [symbol] | −0.122 | −0.358 | 1.597 | 0.099 | −1.347 | 1.039 | 1.946 | 1.154 | −1.628 | 0.158 | 0.247 | −1.24 | −1.958 | −0.645 | 0.306 | 1.046 |
| SAE1 | 695822II [symbol] | −0.782 | −0.386 | −0.314 | −0.923 | −1.055 | −1.02 | −0.983 | −0.0965 | −1.393 | −1.118 | −0.314 | −1.365 | −0.901 | −0.935 | −0.721 | −0.804 |
| SALL2 | 696484II [symbol] | −0.371 | −0.214 | −1.203 | −0.483 | 0.464 | 0.271 | −1.025 | −0.922 | 0.8865 | −0.329 | −0.884 | 1.087 | 0.9135 | 0.072 | 0.345 | −0.844 |
| SARA1 | 690066II [symbol] | −0.092 | 0.435 | 0.874 | 0 | −0.232 | 0.447 | 0.594 | 0.485 | 0.2495 | −0.026 | 0.318 | −0.395 | −0.4215 | −0.302 | 0.023 | −0.131 |
| SARA2 | 688945II [symbol] | −0.176 | −0.058 | 0.134 | 0.058 | 0.116 | −0.464 | −0.637 | −0.5915 | 0.8635 | −0.25 | 0.2 | −0.322 | −0.3685 | −0.137 | −1.09 | 0.567 |
| SAS | 697574II [symbol] | −1.735 | −0.422 | −0.463 | 2.012 | 1.254 | −0.097 | 0.226 | 1.315 | −0.3085 | 1.66 | 1.075 | −0.4 | −1.617 | −1.524 | −0.181 | 0.097 |
| SCIN | 698329II [symbol] | −0.036 | 0.748 | 1.148 | 0.693 | 0.844 | 2.26 | 0.868 | 0.574 | 0.9715 | −0.013 | 2.66 | 0.664 | 1.0155 | 1.563 | 0.362 | 0.705 |
| SCML1 | 683929II [symbol] | 0.9705 | 0.5515 | −1.38 | 1.056 | −0.4645 | −0.2415 | 0.5716 | 0.144 | −0.0545 | 1.662 | 0.9455 | 0.1615 | 1.707 | −0.9315 | −0.1375 | 0.0545 |
| SCPEP1 | 689318II [symbol] | 0.8218 | 0.0938 | 0.7788 | −0.6312 | 0.0808 | −0.5232 | 1.332 | 0.3503 | −1.347 | −0.6052 | −0.1232 | −0.1552 | −0.0008 | −1.01 | −0.6732 | 0.5888 |
| SCRIB | 687588II [symbol] | 0.4937 | 0.6047 | −0.2293 | −0.3563 | −0.3593 | −0.4323 | 0.1047 | 0.6042 | −0.0128 | −0.0913 | 0.0188 | 0.4547 | 0.2297 | −0.3413 | −0.2413 | 0.3747 |
| SDC1 | 681259II [symbol] | −1.838 | 0.2385 | 0.7375 | −1.464 | −1.165 | 0.5805 | 0.2925 | 1.95 | −1.29 | −2.033 | 0.0945 | 0.2365 | −0.329 | −1.277 | 1.274 | 1.293 |
| SDC3 | 693165II [symbol] | 0.382 | 1.427 | 1.063 | 1.535 | 0.398 | −0.695 | 1.046 | 1.535 | 1.9885 | 0.494 | 0.462 | 1.245 | −0.0195 | 0.748 | 0.639 | 0.655 |
| SDCCAG [symbol] | 690950II [symbol] | −0.58 | −1.253 | −0.654 | 3.036 | 0.203 | −0.461 | −0.176 | 0.257 | −0.546 | −0.7 | −0.625 | 0.812 | −0.492 | −0.179 | 0.017 | 0.01 |
| SEC61G | 693650II [symbol] | −0.644 | −0.994 | −0.521 | −0.519 | −1 | −0.393 | −0.315 | −0.596 | −1.756 | −0.507 | 0.351 | −1.054 | −0.7185 | −0.707 | −0.682 | −0.615 |
| SECP43 | 694767II [symbol] | −0.732 | 0.037 | 0.718 | −0.901 | −0.368 | −0.312 | −0.114 | 0.963 | 0.093 | −0.263 | −0.172 | −0.309 | −0.7915 | −0.738 | −0.572 | −0.263 |
| SEDLP | 693304II [symbol] | −0.499 | −0.388 | −0.827 | −0.692 | 0.02 | −0.144 | −0.321 | 0.2755 | 0.6265 | 0.925 | 0.871 | 0.337 | 0.2565 | 0.373 | −0.554 | 0.743 |
| SEMA3B | 696622II [symbol] | −0.828 | 0.412 | −1.215 | 0.465 | −0.081 | −0.704 | −0.164 | −1.271 | 2.1895 | −0.78 | 0.107 | 1.732 | −0.4205 | 1.633 | 0.936 | −1.306 |
| SEMA3C | 691613II [symbol] | −1.5 | 0.7335 | 0.3805 | 1.851 | 0.7245 | −0.0355 | −1.885 | −1.264 | 0.4455 | 1.036 | 0.7395 | −0.0435 | 0.61 | 1.233 | −0.1525 | −0.2305 |
| SEPW1 | 683289II [symbol] | −0.425 | 0.296 | −0.49 | −0.074 | 0.257 | 0.04 | 0.407 | 0.9 | 0.0655 | −0.302 | −0.1 | −0.008 | 0.15 | −0.504 | −0.178 | −0.299 |
| SERP1 | 690913II [symbol] | −0.1627 | −0.2027 | −0.4747 | 0.0933 | 0.0293 | −0.6987 | −0.4707 | −0.0063 | −0.4202 | 1.149 | 0.1713 | 0.4243 | 0.7278 | 0.4613 | −0.8057 | 0.2113 |
| SERPINA [symbol] | 692607II [symbol] | −1.499 | 1.459 | −2.13 | −1.219 | 1.049 | −1.614 | 0.474 | −2.146 | −1.129 | 0.077 | −0.699 | −0.088 | −0.882 | −2.187 | 0.041 | −1.215 |
| SERPINB [symbol] | 683845II [symbol] | −0.0165 | −1.068 | 1.439 | −0.1275 | −0.2735 | −0.9315 | 0.2535 | −0.6935 | 0.059 | −0.1285 | 0.1775 | −0.4705 | −0.1565 | −0.4785 | −0.0325 | −0.1665 |
| SFRP1 | 690138II [symbol] | 3.03 | 0.194 | −0.254 | 1.65 | −0.731 | −0.157 | 2.85 | −0.56 | −0.014 | 0.611 | −0.656 | −0.396 | −0.499 | 0.163 | 0.121 | 2.74 |
| SFRS6 | 689301II [symbol] | 0.0235 | 0.2855 | −0.4105 | −0.2925 | 0.5435 | 0.0615 | 0.3935 | −0.4175 | 0.31 | 0.3665 | −0.0235 | −0.0325 | −0.111 | −0.0335 | −0.1645 | −0.0905 |
| SGCD | 694761II [symbol] | 0.417 | 0.632 | −0.385 | 1.728 | −0.371 | −0.761 | −0.847 | 0.554 | −0.101 | −0.943 | 0.634 | −0.231 | 0.0905 | 0.054 | 0.453 | −0.584 |
| SGCE | 685105II [symbol] | 0.125 | −0.571 | 0.727 | 0.145 | −0.267 | 0.014 | 0.545 | −0.3585 | −1.234 | −0.014 | 0.093 | −0.265 | −0.1865 | −0.817 | −0.684 | 0.196 |
| SH3BGRL | 694455II [symbol] | 0.635 | 2.187 | −0.143 | 0.131 | 1.899 | −0.343 | −0.881 | −1.1965 | −0.037 | 2.126 | 2.111 | 2.096 | 2.123 | 1.239 | −0.037 | 0.667 |
| SHFM1 | 683462II [symbol] | −1.079 | −0.2225 | 0.3965 | −0.2675 | 0.0805 | 0.0655 | 0.0885 | 0.122 | −0.4975 | −0.6485 | 0.5085 | −0.0485 | 0.4685 | −0.4465 | −0.0135 | −0.3395 |
| SIAT7B | 695595II [symbol] | −0.293 | −1.188 | −0.148 | −0.029 | −0.963 | −0.796 | 0.732 | −1.9165 | 0.493 | 1.75 | −0.933 | 0.449 | 0.7785 | −1.118 | 0.697 | −0.554 |
| SIGLEC12 | 690532II [symbol] | −0.0315 | −0.0545 | −0.0595 | −0.1235 | −0.0695 | 0.6705 | 0.2415 | 0.656 | 0.939 | −0.4175 | −0.2975 | 0.2235 | 0.284 | −0.0315 | −0.4165 | 0.0185 |
| SIT | 692124II [symbol] | 1.244 | 0.292 | 0.692 | 0.736 | 0.2 | 1.132 | 0.453 | 1.367 | 0.499 | 0.462 | 0.358 | −0.907 | −0.1995 | −0.462 | −0.399 | 0.883 |
| SITPEC | 681337II [symbol] | 0.3725 | 0.1085 | 0.2365 | 0.3395 | 0.0235 | 0.5895 | −0.1425 | 0.938 | 0.1165 | −1.091 | −0.8955 | −0.0655 | 0.92 | 0.6835 | −0.0595 | −0.4145 |
| SKP1A | 684965II [symbol] | −0.612 | −0.101 | −0.56 | 0.685 | 0.382 | −0.689 | −0.681 | −1.029 | −0.655 | 0.805 | 0.803 | 0.354 | 0.119 | 0.775 | 0.1 | 0.147 |
| SLC12A9 | 696704II [symbol] | 0.764 | 0.683 | 0.225 | 0.747 | 0.514 | 0.791 | 0.073 | 0.49 | 0.1825 | 0.546 | 0.602 | 0.702 | 0.9385 | 0.473 | 0.852 | 0.25 |
| SLC13A3 | 689746II [symbol] | −0.2875 | −0.0615 | −0.6555 | −0.1865 | −0.7335 | 0.7385 | 1.026 | 0.7035 | 0.093 | 0.9515 | −0.2735 | −1.166 | −1.139 | −0.7805 | −0.2765 | 0.0615 |
| SLC16A6 | 687056II [symbol] | −0.0285 | 0.7725 | −1.199 | −0.1135 | 0.1435 | −0.3695 | −0.6765 | −0.961 | 0.61 | 2.965 | 0.0535 | 3.511 | 0.056 | 0.0285 | 0.0805 | 1.934 |
| SLC19A2 | 693146II [symbol] | −0.727 | −0.154 | −2.035 | −0.402 | 1.457 | −0.867 | −1.4 | −1.2115 | 1.9825 | 0.882 | 0.804 | 2.102 | 1.518 | −0.439 | 1.428 | 0.571 |
| SLC1A1 | 692391II [symbol] | 0.369 | 0.046 | 0.698 | −0.118 | 0.5 | 0.557 | 0.32 | 1.8985 | 1.042 | −0.146 | 0.531 | 0.527 | 0.432 | 0.762 | 0.254 | 0.869 |
| SLC1A2 | 690726II [symbol] | −0.2875 | 0.3885 | −0.0055 | 0.0805 | 0.9495 | 0.0055 | −0.9025 | −1.679 | −0.603 | 0.8365 | −0.3925 | 0.8825 | −0.3965 | 5.283 | −0.1795 | −0.4765 |
| SLC24A3 | 689570II [symbol] | 0.3175 | 2.498 | −1.445 | −1.484 | −0.8395 | 0.5385 | 1.264 | 0.589 | −0.4255 | −1.8 | 1.602 | 1.662 | 1.511 | −1.31 | 1.223 | 0.6215 |
| SLC25A2 [symbol] | 692129II [symbol] | 0.19 | −0.271 | 0.712 | 0.6 | 0.99 | 1.585 | 0.426 | 0.456 | 0.725 | 0.699 | 0.405 | 0.602 | 0.5565 | −0.209 | 1.502 | −0.047 |
| SLC25A4 | 684980II [symbol] | −0.3145 | 0.1645 | 0.5085 | 0.9445 | −0.2735 | −1.056 | 0.1205 | −0.481 | 0.25 | 1.25 | −0.1295 | −0.2505 | −2.792 | 0.9725 | −0.4995 | 0.3985 |
| SLC25A5 | 681570II [symbol] | −0.162 | −0.024 | 0.759 | −0.042 | −0.76 | −0.026 | −0.177 | −0.8325 | −1.478 | 0.083 | 0.612 | 0.188 | 0.278 | −0.194 | −0.067 | 0.679 |
| SLC27A2 | 686936II [symbol] | −0.8787 | 0.0343 | 0.3593 | 3.513 | 0.6073 | −0.1027 | −1.122 | −0.8377 | 0.6968 | 1.405 | −0.8227 | 2.064 | −0.0343 | 5.006 | −1.203 | 0.4353 |
| SLC27A5 | 697266II [symbol] | −1.107 | −0.427 | −0.687 | −1.183 | −0.734 | −0.261 | −0.615 | −0.5425 | −0.5215 | −1.244 | −0.148 | 0.096 | 0.7765 | 0.489 | −0.923 | −0.547 |
| SLC30A8 | 695934II [symbol] | 0.011 | −0.117 | 0.264 | 3.102 | 0.533 | −0.063 | −0.077 | −0.1435 | −0.069 | 0.41 | 0.285 | −0.301 | −0.107 | 0.081 | 0.258 | −0.041 |
| SLC39A4 | 684775II [symbol] | −0.26 | 0.574 | −0.251 | −0.796 | −0.502 | −0.457 | 0.186 | 0.3545 | 0.8195 | 0.287 | −0.188 | 0.333 | 0.281 | −0.436 | −0.112 | 0.629 |
| SLC39A6 | 690413II [symbol] | 0.9635 | 0.6375 | −2.047 | 1.77 | 1.972 | −1.003 | −2.988 | −2.199 | −0.5245 | 2.952 | 0.7645 | 2.594 | 2.748 | 1.68 | 2.469 | −0.9125 |
| SLC40A1 | 695661II [symbol] | 3.495 | 3.509 | 1.832 | 2.178 | 1.63 | 2.072 | 0.241 | 1.942 | 4.3185 | 3.945 | 2.542 | 1.602 | 4.899 | 2.767 | 3.17 | 3.376 |
| SLC41A3 | 682343II [symbol] | −0.0915 | 0.0365 | −0.5935 | 0.2155 | −0.0555 | 0.1395 | 0.1515 | 0.198 | −0.1785 | −0.2285 | −0.5935 | −0.5175 | −0.1095 | 0.3485 | −0.3805 | −0.2075 |
| SLC5A6 | 688366II [symbol] | 0.083 | −0.357 | 0.397 | −1.065 | −0.988 | 0.003 | 1.004 | −0.2315 | −0.5995 | −1.041 | −0.068 | 0.227 | −0.8975 | −1.181 | −0.059 | 0.344 |
| SLC6A4 | 691977II [symbol] | −0.1 | 1.623 | 0.03 | −0.038 | −0.186 | 0.103 | 0.049 | −0.1815 | 0.3365 | −0.234 | −0.255 | 0.026 | 2.96 | −0.568 | −0.099 | −0.246 |
| SLC9A3R [symbol] | 682747II [symbol] | −0.129 | 1.076 | 0.426 | −0.007 | 0.367 | −0.583 | −0.158 | −0.9525 | −1.197 | 3.245 | 0.978 | 0.087 | −0.1065 | −0.107 | 1.104 | −0.865 |
| SLITRK6 | 685643II [symbol] | −0.927 | −0.125 | 0.851 | −0.284 | −0.567 | 0.093 | 0.227 | −0.096 | −1.086 | −0.208 | 1.01 | −0.387 | 0.263 | −0.791 | 0.296 | 0.585 |
| SMA3 | 687200II [symbol] | −0.0025 | 0.4805 | −0.3285 | −1.734 | 0.8885 | −1.353 | −1.038 | −1.133 | −0.1015 | 0.2785 | 1.254 | 3.863 | −0.326 | −0.1045 | 0.6355 | 1.252 |
| SMAD2 | 695457II [symbol] | −1.034 | −0.093 | −0.182 | −0.443 | 0.21 | −0.217 | −0.74 | −0.086 | −0.5625 | −0.586 | −0.243 | 0.118 | −0.1125 | −0.256 | −0.266 | −0.482 |
| SMO | 686958II [symbol] | 0.166 | −0.677 | −1.029 | −0.039 | −0.412 | 1.094 | 0.57 | 0.7295 | 0.5625 | −1.019 | −0.343 | −0.154 | −0.282 | −0.973 | 0.382 | −0.186 |
| SMU1 | 689287II [symbol] | 0.154 | 0.112 | −0.717 | −0.562 | 0.34 | 0.122 | −0.1 | −0.1425 | −0.287 | 0.118 | 0.814 | −0.014 | −0.539 | 0.194 | −0.515 | 0.18 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| SNPH | 684407II [symbol] | 0.2778 | −0.0123 | −0.2082 | 0.7498 | 0.1628 | 0.6428 | 0.3058 | −0.0543 | 1.131 | 0.9268 | 0.8578 | −0.0023 | 0.0023 | 0.5818 | −0.1612 | 0.2988 |
| SNRPB2 | 696839II [symbol] | −1.05 | −0.344 | −1.279 | −0.988 | −1.304 | −0.656 | −0.218 | −0.946 | −1.319 | −0.589 | −0.217 | −0.657 | −0.368 | −1.076 | −0.567 | −0.564 |
| SNRPD1 | 695576II [symbol] | −1.959 | −1.547 | −0.887 | −1.163 | −1.763 | −0.94 | −0.388 | −0.8435 | −1.4615 | −1.81 | −1.386 | −1.353 | −0.896 | −1.365 | −1.218 | −1.493 |
| SNRPD3 | 692082II [symbol] | −0.8965 | −0.5555 | 1.201 | 0.1705 | 0.0095 | −0.2235 | 0.1225 | −0.095 | −0.2385 | 0.0345 | 0.3195 | −0.0565 | −0.5095 | −0.1995 | −0.7185 | 0.3415 |
| SNRPG | 684507II [symbol] | −0.952 | −0.34 | 0.867 | −0.508 | 0.093 | −0.255 | 0.432 | 0.0635 | 0.0395 | 0.131 | 0.698 | −0.003 | −0.726 | −0.442 | −0.277 | 0.279 |
| SNX7 | 685968II [symbol] | 0.7175 | 0.0465 | 0.2505 | −0.2105 | −0.7775 | −0.6105 | 0.1505 | 0.1345 | −0.857 | 0.3525 | 0.6465 | 0.2255 | −0.3565 | 0.0245 | −0.1905 | −0.5325 |
| SOD2 | 684100II [symbol] | −1.135 | −0.6397 | 1.002 | 0.0053 | 0.4643 | −0.1687 | 1.734 | −0.0053 | −1.638 | −0.0978 | −0.0198 | −0.1617 | −1.393 | −0.5777 | −1.246 | 1.15 |
| SORCS1 | 690320II [symbol] | 0.3705 | 0.4475 | 0.0255 | 0.1115 | −0.0505 | −0.5305 | −0.5165 | −0.561 | 0.2655 | −0.8815 | −0.4195 | 0.5525 | 0.981 | −0.0975 | 0.5965 | −0.1925 |
| SOX4 | 686301II [symbol] | −0.5885 | 0.0665 | 0.5645 | −0.6715 | −0.1065 | 1.282 | 1.419 | 1.768 | 1.093 | −0.3925 | 0.8675 | 0.0005 | 0.2905 | −0.3745 | 1.347 | −0.5195 |
| SOX9 | 681418II [symbol] | −0.624 | −0.005 | 0.476 | 0.686 | 1.572 | 0.387 | 0.386 | 0.083 | 0.0865 | 1.516 | 0.077 | 1.092 | 0.767 | 0.024 | 0.729 | 0.367 |
| SPAG16 | 687462II [symbol] | −0.8592 | 0.1978 | 0.7328 | −0.2902 | 0.0128 | 1.021 | 0.9008 | 0.3478 | −0.4772 | −1.134 | −0.0833 | −0.0653 | −0.0128 | −0.6412 | −0.0703 | −0.1202 |
| SPAG5 | 686120II [symbol] | −1.389 | 0.6765 | 1.152 | −0.6085 | −0.6105 | 0.0685 | 0.5695 | 0.037 | −0.747 | −0.3415 | −0.0345 | −0.6295 | −0.2705 | −0.9175 | 0.1315 | −0.3365 |
| SPC18 | 695899II [symbol] | −1.731 | −1.098 | −0.78 | −1.48 | −1.646 | −1.701 | −1.44 | −2.175 | −2.817 | −1.81 | −1.252 | −1.218 | −1.9775 | −1.237 | −1.234 | −1.289 |
| SPFH2 | 686593II [symbol] | −1.6 | −0.013 | −0.592 | 0.543 | 0.586 | 0.67 | 0.044 | 0.111 | −0.202 | −0.2 | 0.588 | 0.449 | −0.157 | 0.852 | 0.268 | −0.434 |
| SPG3A | 685052II [symbol] | −0.349 | 0.774 | 0.367 | 0.899 | 0.857 | −0.57 | 0.043 | −0.191 | −0.2185 | 0.272 | 0.096 | −0.271 | 0.5485 | 1.123 | 0.612 | −0.22 |
| SPPL3 | 684761II [symbol] | −0.4465 | 0.3005 | 0.0125 | 0.1695 | 0.1955 | −0.2255 | 0.0845 | 0.096 | −0.441 | 0.0325 | −0.3835 | −0.3315 | 0.052 | −0.3905 | 0.1165 | −0.0885 |
| SQLE | 765400II [symbol] | −2.217 | −1.5 | −0.52 | −1.743 | −2.06 | 0.458 | −2.212 | −1.3925 | −2.8535 | −1.82 | −1.925 | −2.071 | −2.2235 | −2.928 | −2.27 | 0.227 |
| SQRDL | 693902II [symbol] | −0.8995 | 0.1085 | 0.3715 | 0.1545 | 0.6805 | −0.0595 | −0.1495 | 0.2085 | 0.2675 | 0.2895 | 0.1725 | −0.0685 | 0.1285 | −0.0315 | −0.2645 | 0.0315 |
| SREBF1 | 697880II [symbol] | −0.051 | 0.972 | 1.06 | 2.221 | 1.86 | 0.897 | 0.515 | 0.6855 | 2.5175 | 2.147 | 1.285 | 1.319 | −0.342 | 2.755 | 0.404 | 0.228 |
| SRI | 698971II [symbol] | −0.576 | −0.669 | −0.118 | −0.435 | −0.77 | −0.011 | 2.125 | 0.4875 | 0.8865 | −0.055 | −0.162 | −0.278 | 0.159 | 0.312 | 1.229 | −0.42 |
| SRPK1 | 697017II [symbol] | −1.607 | −2.113 | −1.003 | −1.337 | −1.535 | −1.344 | −0.505 | −1.876 | −1.7 | −1.137 | −1.052 | −1.436 | −1.899 | −1.507 | −1.994 | −1.156 |
| SSFA2 | 692106II [symbol] | −0.261 | −0.837 | −1.445 | −0.245 | 2.127 | −1.384 | −0.999 | −2.075 | −1.1545 | −0.489 | −0.98 | −0.864 | −1.5005 | 0.96 | −0.877 | −0.469 |
| SSR4 | 686717II [symbol] | 0.949 | 1.737 | −0.457 | 1.912 | 1.674 | 1.132 | 1.87 | 2.056 | 2.0695 | 2.665 | −0.56 | 2.616 | 1.7255 | 2.723 | 2.13 | 1.035 |
| STARD3N | 686038II [symbol] | −0.008 | 0.043 | 0.337 | −0.263 | −0.434 | −0.714 | −1.403 | −0.507 | −0.913 | 0.5 | 0.677 | 0.383 | −0.0425 | −0.048 | −0.028 | 0.206 |
| STARD7 | 681306II [symbol] | 0.2655 | 0.2525 | 0.3085 | 0.4385 | −0.1175 | 0.1735 | −0.4065 | −0.159 | −0.8935 | 0.1725 | 0.2635 | −0.2535 | 0.038 | 0.3455 | 0.1415 | 0.5445 |
| STAT1 | 681492II [symbol] | 0.0785 | 0.9275 | 0.0425 | 0.7285 | −0.6415 | 0.2365 | −0.1675 | −0.0615 | −2.148 | 1.576 | 0.8495 | −0.3075 | −1.256 | −0.4615 | −0.5455 | 0.7575 |
| STC2 | 689625II [symbol] | 1.065 | 3.109 | 0.2735 | 2.306 | 2.91 | −1.04 | −0.2195 | −0.764 | 1.696 | 2.185 | −1.1 | −1.015 | 1.748 | −0.7605 | 1.205 | −0.6795 |
| STK24 | 688430II [symbol] | 1.279 | −0.0345 | −1.033 | 0.9055 | 0.1285 | 0.2185 | −1.642 | −1.136 | −0.7675 | −0.0875 | −0.7465 | 0.1185 | 0.626 | 0.8065 | −0.3795 | 0.4785 |
| STK6 | 685902II [symbol] | −0.399 | 0.075 | 0.709 | −0.677 | −0.612 | −0.206 | 0.34 | −0.123 | −0.6765 | −0.302 | 0.425 | −0.636 | −0.476 | −0.69 | 0.133 | −0.047 |
| STMN1 | 682764II [symbol] | −0.8595 | 0.5675 | 1.746 | −0.6945 | −0.2665 | 2.489 | 1.393 | 0.391 | −1.017 | 0.0395 | 2.005 | −0.2435 | −0.04 | −0.5135 | −0.6495 | −0.5555 |
| STX1A | 691816II [symbol] | −0.465 | 0.361 | 0.116 | −0.605 | −0.073 | 0.203 | −0.228 | 0.5925 | −0.108 | −0.505 | 0.284 | 0.22 | 1.05 | 0.817 | −0.344 | −0.609 |
| STX3A | 691051II [symbol] | −0.8805 | −0.0635 | −0.2845 | 0.0045 | 0.3225 | 0.2625 | 0.3215 | −0.032 | 0.403 | 0.0915 | −0.2995 | −0.0045 | −0.8405 | 0.0265 | −0.5315 | 0.2155 |
| STYX | 685620II [symbol] | 0.439 | −0.369 | −0.12 | −0.317 | −0.207 | 0.053 | −0.358 | −0.488 | −0.179 | 0.506 | 0.768 | 0.831 | −0.1555 | −0.366 | 0.18 | 0.279 |
| SUGT1 | 693218II [symbol] | −1.212 | −0.867 | −0.737 | −0.185 | −0.249 | 0.49 | 0.453 | 0.0085 | 0.4855 | 0.04 | 0.198 | −0.449 | −0.817 | 0.167 | −0.263 | −0.277 |
| SUMF1 | 695843II [symbol] | −0.0045 | 0.7065 | 4.265 | −0.2925 | −0.1675 | 3.317 | 2.524 | −0.275 | −0.306 | −1.211 | −0.4665 | 4.152 | −0.552 | −1.196 | 0.0045 | 0.6095 |
| SUMO2 | 689403II [symbol] | −0.666 | −0.46 | 0.417 | −0.024 | −0.4 | −0.307 | 0.732 | −0.478 | −1.044 | 1.268 | −0.017 | 0.162 | −0.374 | 0.248 | 0.058 | −0.256 |
| SUV39H2 | 681342II [symbol] | 0.223 | −0.638 | 0.567 | −0.016 | −1.038 | −0.041 | 1.42 | −0.06 | 0.0145 | −0.296 | 0.577 | −0.001 | 0.114 | −0.521 | −0.819 | 0.245 |
| SYAP1 | 681813II [symbol] | 0.069 | −0.015 | 0.24 | 0.003 | 0.573 | −0.342 | −0.003 | −1.094 | −0.428 | 1.387 | 0.735 | −0.079 | −0.212 | 0.452 | 0.089 | −0.205 |
| SYCP2 | 689491II [symbol] | −0.598 | −1.098 | −3.181 | 0.998 | 1.29 | −0.512 | 0.056 | 0 | −1.394 | 1.856 | 0.826 | 1.241 | 0.2105 | −0.805 | −0.797 | 1.539 |
| SYNGR1 | 686385II [symbol] | −0.0485 | −0.3495 | 0.8185 | −0.7885 | −0.9925 | 0.7645 | −0.6565 | 0.376 | 0.381 | −1.019 | −0.5735 | 0.1745 | −0.289 | −0.8265 | −0.3355 | 0.5395 |
| SYTL2 | 692920II [symbol] | −0.797 | 0.55 | −0.128 | 1.142 | 0.877 | −1.655 | −1.411 | −0.9045 | 0.621 | 1.442 | 0.708 | −0.541 | 0.848 | 0.495 | 0.937 | −0.695 |
| TAF1B | 681153II [symbol] | −0.364 | 0.002 | −0.714 | −0.327 | 0.348 | −0.175 | 0.51 | −0.3905 | −0.4765 | 0.401 | −0.086 | 0.157 | −0.739 | 0.309 | 0.082 | 0.327 |
| TAF6 | 687144II [symbol] | −0.372 | 0.334 | −0.067 | −0.59 | 0.254 | −0.057 | 0.021 | 0.125 | −0.0375 | −0.184 | 0.837 | 0.742 | 0.3675 | −0.207 | 0.073 | −0.205 |
| TARBP1 | 697518II [symbol] | 0.301 | −0.078 | −0.939 | 0.009 | 1.395 | −1.38 | 0.586 | −0.76 | −0.3145 | 0.16 | −0.709 | −0.585 | 0.6035 | −0.688 | −0.25 | −0.181 |
| TARS | 686090II [symbol] | −0.834 | −0.199 | 0.464 | 0.163 | −0.162 | 0.068 | 0.896 | 0.158 | −1.006 | −0.213 | 0.444 | −1 | −0.8275 | −0.068 | −0.905 | 0.861 |
| TATDN1 | 692620II [symbol] | −1.297 | −0.212 | 0.34 | −0.867 | −0.124 | 0.357 | 0.613 | −0.063 | 0.229 | −0.153 | 0.116 | −0.21 | −0.8815 | −0.037 | −0.224 | 0.662 |
| TBC1D12 | 689394II [symbol] | −0.9205 | 0.0365 | 0.4355 | −0.8615 | −0.4295 | −0.1995 | 1.267 | −0.54 | −0.343 | 0.0825 | 0.9455 | 0.3595 | −0.2785 | −0.2105 | −0.4815 | −0.0205 |
| TBC1D14 | 683294II [symbol] | 0.4328 | −0.6892 | −1.582 | 0.4478 | 0.6088 | −0.3772 | −1.039 | −0.0138 | 0.0158 | 0.1128 | −0.1162 | 0.7928 | −0.5787 | −0.0943 | −0.4952 | 0.9948 |
| TBC1D3 | 684780II [symbol] | 0.008 | −0.335 | −0.579 | −0.484 | −0.218 | 0.104 | 0.441 | −0.3465 | −0.4745 | 0.404 | −1.03 | −0.15 | 0.281 | −0.585 | −0.1296 | 0.883 |
| TBCA | 689449II [symbol] | −0.8765 | −0.3795 | 0.1675 | 0.6835 | −0.0025 | −0.5715 | −0.3525 | −0.627 | −0.6225 | 0.7855 | 0.5245 | −0.1445 | −0.3885 | 0.2545 | 0.2005 | 0.5465 |
| TBX1 | 682010II [symbol] | −0.1465 | 0.4835 | −1.273 | −0.4425 | 0.9785 | −0.2185 | 1.222 | −0.1945 | 1.987 | −0.5735 | −0.0055 | −0.9445 | −0.8985 | −0.3195 | 0.0055 | 1.338 |
| TBX3 | 687903II [symbol] | 0.575 | 0.694 | −1.662 | 0.986 | −1.504 | −1.488 | −2.057 | −0.871 | −0.728 | 1.564 | 1.17 | 1.485 | −0.7745 | 0.857 | 1.331 | −0.576 |
| TCAP | 680995II [symbol] | 0.0925 | −0.2795 | −0.0005 | −0.5225 | −0.3555 | 2.108 | 0.4845 | 3.644 | 0.3625 | 0.0005 | −0.4485 | −0.0695 | −0.1555 | −0.4535 | 0.0885 | 2.31 |
| TCEAL1 | 688759II [symbol] | −0.0735 | 0.3855 | 0.0765 | 1.744 | 0.1815 | −0.5945 | −0.8455 | −1.156 | −0.2725 | 1.469 | 0.0735 | 0.6775 | 0.457 | 1.422 | 0.1075 | −0.1055 |
| TCEAL3 | 691252II [symbol] | 0.385 | −0.344 | −0.522 | 0.045 | −0.759 | 0.735 | 0.825 | 2.027 | −0.162 | 0.025 | 0.175 | 0.085 | −0.442 | 0.205 | 0.106 | 0.225 |
| TCEAL4 | 687353II [symbol] | −0.582 | −0.2 | −0.423 | 0.511 | 0.612 | −0.134 | 0.038 | −0.4995 | −0.1205 | 2.662 | −0.535 | −0.411 | −0.007 | 1.051 | 0.007 | 0.296 |
| TCF15 | 685545II [symbol] | 0.0795 | −0.1695 | 0.3085 | −0.9575 | 0.1105 | 0.2255 | 0.7855 | 1.544 | −0.514 | −1.655 | −0.5845 | 0.4005 | −0.416 | −1.542 | −0.2085 | 0.1585 |
| TCF3 | 689758II [symbol] | 0.416 | 0.56 | 1.359 | −0.569 | −0.475 | 0.705 | 2.37 | −0.104 | 0.1615 | −0.488 | 0.234 | 1.132 | −0.416 | −0.262 | −0.033 | 0.082 |
| TCF7L2 | 688358II [symbol] | 0.0005 | 0.1045 | −0.0215 | −0.6005 | 0.5535 | 0.0365 | −0.0495 | 0.1415 | −0.5705 | −0.3265 | −0.5415 | −1.228 | −0.1325 | −0.3155 | 0.6095 | 0.2455 |
| TDRD7 | 695761II [symbol] | −0.777 | 0.093 | 0.617 | 0.612 | 0.365 | −0.186 | 0.096 | 0.907 | −0.0675 | 1.689 | 0.539 | −0.337 | −0.4695 | 0.038 | −0.141 | 0.411 |
| TESK2 | 685924II [symbol] | 1.606 | −0.0703 | 0.2758 | 0.8428 | −0.6132 | −0.0353 | −0.5532 | −0.5052 | 0.0263 | 0.2988 | −0.5102 | 2.5 | −0.9822 | −0.4442 | 0.0748 | 0.1278 |
| TFAP2B | 686514II [symbol] | −0.114 | 0.954 | 1.476 | −0.734 | −4.08 | 2.163 | −4.236 | 1.221 | −1.342 | −2.568 | −3.686 | 0.505 | 1.098 | 0.885 | 0.529 | 0.524 |
| TFCP2L1 | 685915II [symbol] | 1.4 | 0.394 | −0.008 | 0.049 | −0.464 | −1.324 | 2.28 | −0.909 | 0.008 | 1.391 | −0.127 | −0.82 | −0.849 | −0.092 | −0.667 | 0.398 |
| TFCP2L2 | 697960II [symbol] | 1.325 | 1.469 | 0.788 | −0.4 | 1.68 | 1.178 | 2.825 | 1.306 | 1.2125 | 0.46 | 1.688 | 1.908 | 0.894 | 0.303 | 1.865 | 1.906 |
| TFF1 | 688687II [symbol] | −0.145 | −0.233 | 0.846 | 0.003 | −0.114 | −0.315 | 0.876 | 1.319 | −0.516 | −1.093 | −0.512 | −0.165 | −0.4085 | −0.285 | 0.524 | 0.009 |
| TFF3 | 695605II [symbol] | 0.301 | 0.985 | 1.01 | 0.04 | 0.122 | 2.347 | 0.49 | 1.3365 | 0.5245 | 1.84 | 1.847 | 2.496 | 0.0895 | 2.366 | 0.47 | 4.335 |
| TFRC | 693432II [symbol] | −2.03 | −2.123 | −0.85 | −2.501 | −3.568 | −2.153 | −2.46 | −2.1945 | −3.2305 | −2.427 | −1.773 | −2.321 | −1.7495 | −2.804 | −2.887 | −0.578 |
| TGFBR3 | 690641II [symbol] | 1.321 | 0.438 | −0.753 | −0.612 | 2.647 | 0.027 | −0.512 | −0.836 | −0.54 | 0.009 | −0.211 | 1.052 | −0.6425 | 0 | −0.273 | 0.639 |
| THY28 | 687185II [symbol] | −1.087 | 1.695 | 1.316 | −1.017 | −0.616 | 2.015 | 1.313 | 1.3725 | −0.1215 | −0.757 | 0.393 | −0.459 | 0.0555 | 0.317 | 0.452 | 1.005 |
| TIF1 | 682191II [symbol] | −0.7645 | 0.3025 | 0.2075 | −0.0155 | −0.5115 | 0.7255 | 0.0615 | −0.3445 | 0.4885 | 0.2015 | 0.2895 | −0.0265 | 0.3885 | −0.7005 | 0.2695 | −0.6025 |
| TIGA1 | 689331II [symbol] | 0.8035 | −0.0825 | −0.9185 | 0.7155 | 0.7795 | −0.0415 | −1.14 | −0.554 | 0.3455 | 0.0415 | −0.1465 | 0.0485 | 0.8315 | 0.7455 | 0.2105 | 0.3355 |
| TIM14 | 681121II [symbol] | −0.429 | −0.599 | −0.823 | 0.337 | 0.154 | −1.119 | −0.689 | −1.219 | −0.5855 | 0.444 | 0.194 | 0.206 | 0.1625 | 0.814 | −0.443 | 0.088 |
| TIMELES [symbol] | 689108II [symbol] | −0.7075 | 0.5005 | 1.071 | −0.1345 | −0.1405 | 0.2615 | 1.108 | 0.1015 | −0.8185 | −0.0375 | −0.2565 | −0.7355 | 0.599 | −0.5105 | −0.0045 | −0.0925 |
| TIMM9 | 684722II [symbol] | −0.518 | −0.076 | −0.797 | −0.042 | 0.331 | 0.309 | −0.076 | −0.5435 | −0.282 | 0.077 | 0.061 | 0.876 | 0.3505 | 0.042 | 0.091 | −0.141 |
| TINP1 | 688051II [symbol] | −0.2278 | −0.0618 | −0.4498 | 0.2012 | 0.6642 | −0.0478 | −1.106 | −0.5563 | 0.1972 | 0.3812 | −0.0588 | 0.1082 | −0.0103 | 0.0233 | −0.3668 | 0.2972 |
| TIP120B | 686908II [symbol] | −0.4065 | 0.1195 | −0.9015 | −0.7165 | 1.236 | −0.2925 | 0.2125 | 0.169 | 2.136 | 1.509 | 1.171 | 0.3635 | 0.7715 | −0.9325 | 1.389 | 0.1415 |
| TK1 | 686069II [symbol] | −1.126 | −1.091 | 1.01 | −0.256 | −0.692 | 0.329 | 1.4 | 2.728 | −0.188 | −0.342 | −0.053 | −0.593 | 0.4565 | −0.687 | −0.867 | −0.115 |
| TLE2 | 690798II [symbol] | 0.0825 | 0.4775 | −0.8075 | 0.0195 | 0.4175 | 0.0155 | −0.0565 | −0.1895 | −0.8375 | −1.111 | 0.0715 | −0.0665 | 0.4935 | −0.5875 | 0.3705 | −0.1255 |
| TLR5 | 682772II [symbol] | −0.3315 | −0.1385 | −0.9965 | −0.8945 | 0.2665 | 1.207 | 0.7365 | 0.0535 | −0.808 | −0.5805 | −0.6425 | 1.296 | 0.099 | −0.3375 | −0.6275 | 1.069 |
| TM4SF12 | 689572II [symbol] | 0.2865 | −0.8155 | −1.819 | 0.9055 | −0.3315 | 0.0415 | 0.7115 | −1.078 | −0.993 | −0.0055 | −0.1625 | −1.327 | −1.232 | 2.204 | 0.6365 | 0.9895 |
| TM4SF13 | 696352II [symbol] | 0.223 | 0.981 | 0.921 | 0.169 | 0.85 | 0.461 | −0.038 | −0.6125 | −0.243 | 0.246 | 1.162 | 1.195 | 1.5455 | 1.197 | 0.532 | 1.426 |
| TMEM14A | 683793II [symbol] | −0.332 | −0.328 | −0.08 | −0.099 | 0.055 | −0.169 | 0.333 | −1.005 | −1.001 | 0.594 | 0.497 | 0.222 | −0.1555 | 0.414 | −0.45 | −0.263 |
| TMEM25 | 697985II [symbol] | 1.485 | 2.092 | 0.811 | 3.405 | 3.363 | 0.784 | 1.458 | 1.449 | 3.859 | 2.418 | 3.497 | 2.931 | 2.224 | 4.014 | 1.985 | 2.73 |
| TMEM4 | 695931II [symbol] | −1.298 | −0.489 | −0.275 | −0.683 | −0.221 | −0.169 | 0.535 | −0.3675 | 0.272 | −0.534 | −0.217 | −0.069 | 0.0645 | 1.003 | −0.324 | −0.041 |
| TMPRSS5 | 689589II [symbol] | 0.355 | −0.2 | 0.044 | 0.279 | 0.264 | 0.127 | 0.144 | −0.044 | 0.5555 | −0.442 | −0.487 | −0.571 | −0.3985 | −0.438 | 0.231 | −0.314 |
| TMSB10 | 695386II [symbol] | −0.796 | −1.333 | −0.146 | 0.236 | −1.109 | 0.056 | 1.893 | 2.428 | −1.521 | −0.886 | −2.318 | −2.276 | −1.998 | 1.545 | −1.604 | −0.201 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| TMSNB | 681721II [symbol] | 0.515 | −0.856 | 0.952 | 0.285 | −1.579 | 1.395 | 1.715 | 0.7005 | 0.339 | 0.055 | 0.075 | −0.085 | 0.2315 | −0.265 | −1.549 | 0.555 |
| TNFRSF1 [symbol] | 681592II [symbol] | 1.105 | −0.671 | −0.601 | 0.869 | 0.857 | 0.707 | −0.049 | 0.2935 | 0.3835 | 0.938 | −0.187 | −0.17 | −0.379 | −1.771 | −0.116 | 0.184 |
| TNFRSF2 [symbol] | 689952II [symbol] | 0.0255 | −1.065 | 0.2195 | −0.3185 | −1.113 | 0.2505 | 0.7175 | −0.5265 | −0.6225 | −0.8125 | 0.1255 | −0.4715 | −0.753 | 0.8655 | −0.0255 | 0.2695 |
| TNFRSF2 [symbol] | 692465II [symbol] | −1.943 | −0.106 | 0.357 | −0.563 | 0.14 | −0.117 | 0.844 | −0.776 | −0.423 | 0.93 | 0.7 | 0.406 | 0.6895 | 0.364 | 0.267 | −0.718 |
| TNFSF4 | 694020II [symbol] | −0.295 | 0.812 | −0.218 | −0.615 | 0.836 | −0.168 | 0.405 | 0.4515 | −0.613 | 0.103 | 2.104 | 0.001 | 0.6525 | 2.358 | 0.323 | 1.134 |
| TNFSF7 | 684958II [symbol] | −0.3295 | −0.1695 | 1.896 | 0.7375 | 0.2305 | 0.1715 | −0.2845 | 2.316 | 0.393 | 3.06 | −0.2025 | 0.2355 | 0.4875 | 0.5015 | −0.6825 | −0.4525 |
| TNIK | 695447II [symbol] | 0.278 | 0.824 | 0.452 | −0.514 | 0.736 | −0.105 | −0.827 | −0.565 | 0.7685 | −0.23 | 1.517 | 0.387 | 0.4545 | 0.107 | 0.426 | −0.552 |
| TNNC2 | 687349II [symbol] | 0.5432 | 0.9222 | 0.1818 | −0.4018 | 1.472 | 0.4622 | 1.25 | 1.355 | 0.0088 | −0.1176 | −0.6648 | −0.3448 | −0.1788 | 0.1272 | 1.06 | 0.0243 |
| TNNI2 | 687679II [symbol] | 0.553 | 2.324 | 1.511 | −0.285 | 0.829 | 1.559 | 0.809 | 0.997 | 2.117 | 1.352 | 2.428 | 1.166 | 1.7275 | 0.043 | 1.256 | 1.527 |
| TNNI3 | 695629II [symbol] | −0.79 | 1.666 | −0.87 | −1.27 | −1.739 | 0.17 | −1.481 | −1.464 | −0.3905 | −1.4 | −0.013 | −0.642 | 2.1455 | −1.158 | 1.684 | −1.846 |
| TOB1 | 683402II [symbol] | −0.0015 | 0.3535 | −0.1455 | −0.2445 | 0.1235 | 0.8465 | −0.2175 | −0.9805 | −0.0875 | 2.088 | 0.0015 | 0.7685 | 0.958 | −0.0165 | 1.242 | 1.302 |
| TOM1L1 | 685320II [symbol] | −1.206 | −1.088 | 0.6535 | −0.7075 | 0.2975 | −1.839 | −0.4255 | −2.015 | −0.538 | 2.358 | 0.2215 | 0.8575 | 0.0635 | −0.2575 | −0.1915 | 0.8075 |
| TOMM7 | 683060II [symbol] | 0.506 | 0.096 | −0.782 | −0.121 | 0.269 | 0.057 | −0.242 | 0.2625 | −0.311 | 0.175 | 0.472 | 1.145 | 0.7535 | 0.073 | 0.352 | 0.404 |
| TOP1MT | 693021II [symbol] | −0.474 | −0.502 | 0.029 | −0.343 | −0.603 | 2.329 | −0.115 | −0.5405 | −0.9695 | −1.172 | −1.029 | 0.162 | 0.0825 | −0.674 | −0.385 | −0.712 |
| TOP2A | 690278II [symbol] | −0.6495 | −0.2105 | 0.7415 | −0.3155 | −0.4035 | 1.236 | −0.1805 | −0.55 | −0.7375 | −0.1945 | 0.1155 | −0.8016 | −0.1465 | −0.8935 | −0.0525 | −0.6545 |
| TOP2B | 687326II [symbol] | −0.055 | 0.461 | −0.44 | 0.204 | 0.553 | 0.17 | −0.37 | −0.547 | −0.6665 | 0.568 | −0.422 | 0.482 | 0.431 | 0.024 | 0.063 | 0.1 |
| TOPBP1 | 697492II [symbol] | −0.945 | −0.767 | 0.218 | −0.664 | −0.847 | −0.479 | 0.25 | −0.605 | −1.486 | −0.324 | −0.276 | −0.451 | −0.3315 | −0.871 | −0.943 | −0.636 |
| TOPK | 684492II [symbol] | −2.06 | −0.04 | 0.003 | −0.131 | −0.781 | −0.142 | 0.506 | −1.133 | −0.805 | 0.115 | 1.259 | −0.078 | 0.222 | −0.843 | −0.411 | −0.755 |
| TP53AP1 | 688947II [symbol] | −0.206 | 1.227 | 0.028 | −0.028 | 0.599 | −0.353 | −0.922 | −0.1495 | 1.607 | 0.57 | 0.727 | 1.872 | 1.5 | −0.306 | 0.697 | −0.394 |
| TP53BP2 | 681831II [symbol] | −0.0085 | −1.026 | −0.6335 | −1.278 | 0.3755 | 0.2955 | 0.6075 | 0.5225 | −0.3095 | −1.208 | 0.0125 | 0.7755 | 0.339 | −1.6 | −0.4235 | 0.8505 |
| TP53INP1 | 686814II [symbol] | −0.922 | 0.363 | −0.689 | −0.258 | 0.895 | 1.383 | −0.626 | −0.234 | −0.2395 | 1.694 | −0.002 | 0.942 | 1.019 | −0.25 | −0.041 | 0.885 |
| TPD52L1 | 684176II [symbol] | −0.0855 | −0.0655 | −0.0245 | 0.1385 | −0.1695 | 0.3065 | −0.0085 | 0.38 | 0.086 | −0.1345 | −0.2085 | −0.1065 | 0.105 | 0.3795 | −0.0395 | 0.0085 |
| TPRT | 695363II [symbol] | −0.999 | −1.864 | −0.466 | −1.239 | −1.606 | −1.366 | −0.121 | −1.2425 | −1.871 | −0.708 | −1.182 | −1.599 | −1.843 | −1.837 | −1.717 | −0.936 |
| TRA@ | 696810II [symbol] | 0.2055 | 0.8925 | 1.088 | −0.0375 | 1.235 | −0.1145 | 0.1685 | −0.888 | 0.5335 | −0.1745 | 1.362 | 2.038 | 0.177 | −0.1305 | 1.376 | −0.0525 |
| TRAF4 | 695150II [symbol] | 0.464 | 1.971 | 0.64 | 0.635 | 0.248 | 1.941 | 0.994 | 0.841 | 0.8725 | 1.165 | 0.04 | 0.508 | 0.9815 | 0.136 | 0.946 | −0.151 |
| TRAPPC4 | 682554II [symbol] | −0.435 | −0.344 | 0.385 | 0.342 | 0.503 | −0.048 | −0.63 | −0.083 | 0.2115 | −0.144 | 0.554 | −0.337 | 0.3475 | 0.643 | −0.247 | 0.535 |
| TRH | 688633II [symbol] | 0.73 | 0.162 | −0.202 | −0.315 | 0.157 | −0.097 | 0.046 | 0.5465 | 1.864 | 5.493 | −0.06 | 2.527 | −0.077 | 0.441 | 0.103 | −0.375 |
| TRIM17 | 684736II [symbol] | −0.1843 | 0.2567 | −0.7273 | −0.1633 | 0.3577 | −0.0333 | −0.2773 | −0.5623 | 0.3207 | −0.0053 | −0.5463 | −0.5483 | 0.0053 | −0.5843 | −0.5633 | 0.6477 |
| TRIM2 | 682053II [symbol] | 0.9928 | −0.6642 | 0.5608 | 0.5218 | −0.3212 | 0.9428 | 1.619 | −0.0118 | 0.2188 | 0.4068 | −1.121 | −1.134 | −1.231 | 0.5198 | −0.5792 | 1.278 |
| TRIM6 | 682620II [symbol] | −0.5 | 0.78 | 0.35 | 0.185 | 0.025 | −0.637 | −0.046 | 0.482 | −0.443 | −0.412 | 0.213 | −0.053 | −0.2895 | 0.381 | −0.025 | 0.26 |
| TRIP3 | 696359II [symbol] | −0.6 | −1.141 | −0.597 | −0.42 | −0.79 | −1.075 | −0.921 | −0.4005 | −0.6465 | 0.149 | −0.839 | −0.123 | −0.1195 | −0.433 | −0.583 | −0.247 |
| TRPS1 | 683107II [symbol] | 0.1265 | 0.1685 | −1.644 | −0.0685 | 0.6165 | 0.2235 | −0.8665 | −0.709 | −0.3205 | −0.1035 | 0.2255 | 1.037 | −0.387 | 0.7805 | −0.4815 | −0.4885 |
| TSG101 | 685755II [symbol] | 0.1805 | 0.5395 | −0.1245 | 0.4925 | 0.3975 | −0.2715 | −0.2635 | 0.556 | −0.095 | 0.4905 | 0.1985 | −0.0246 | −0.425 | 0.7935 | −0.3285 | −0.0815 |
| TTC10 | 687943II [symbol] | 0.0065 | −0.5145 | −0.7475 | 0.6085 | 0.9545 | 0.7135 | −0.8765 | −0.8075 | −0.2155 | 0.2675 | −0.0065 | 0.8125 | 0.092 | 0.3015 | 0.0515 | −0.2055 |
| TTC12 | 682598II [symbol] | −0.039 | 0.035 | −0.052 | −0.104 | 0.343 | 0.204 | −0.087 | 0.3925 | −0.014 | 0.145 | −0.108 | −0.085 | −0.1105 | 0.314 | 0.049 | 0.168 |
| TTK | 691976II [symbol] | −0.984 | −0.021 | 1.333 | −0.609 | −0.476 | 0.497 | 0.809 | 0.0705 | −0.4235 | −0.639 | 0.408 | −0.061 | 0.021 | −1.333 | −0.682 | −0.543 |
| TTLL4 | 685109II [symbol] | 0.0935 | −0.8135 | 0.2445 | −0.0755 | −0.3835 | 0.0545 | 0.8575 | −1.097 | 0.0035 | −0.3365 | −0.9005 | −0.0595 | −0.3935 | −0.8225 | −0.0035 | 0.0655 |
| TTYH1 | 688941II [symbol] | 0.5985 | −0.2605 | −0.0075 | 0.0075 | −0.1525 | 0.0485 | 0.9245 | 0.0395 | 0.7495 | −0.3695 | −0.4525 | −0.4995 | −0.243 | 0.0875 | −0.0285 | 0.1085 |
| TUBB3 | 685420II [symbol] | −0.174 | −0.17 | 2.285 | −0.167 | −0.461 | −0.058 | −0.006 | 1.107 | −0.2065 | −0.942 | 1.329 | 0.293 | −0.2135 | 0.947 | 0.647 | −0.644 |
| TULP3 | 684931II [symbol] | 0.022 | 0.035 | 0.275 | −0.022 | 0.143 | 1.187 | 0.856 | 0.4425 | −0.1195 | −0.196 | −0.325 | −0.233 | −0.214 | −0.948 | 0.355 | −0.755 |
| TXN | 687357II [symbol] | −1.205 | 0.205 | 1.011 | −1.684 | −0.685 | 1.866 | −0.854 | 0.5685 | −0.996 | −0.195 | 0.346 | 0.565 | 0.1 | −1.365 | −0.826 | 1.805 |
| TYMS | 763590II [symbol] | −4.416 | −2.95 | −1.2 | −2.759 | −3.84 | −2.208 | −2.516 | −3.2415 | −3.292 | −3.218 | −3.499 | −3.084 | −3.153 | −4.199 | −3.76 | −3.059 |
| U5-116KD | 696228II [symbol] | −0.631 | −0.574 | 0.067 | −0.672 | −1.097 | −0.605 | −0.413 | −0.4435 | −0.394 | −0.34 | −0.663 | −0.192 | −0.232 | −0.841 | −0.283 | −0.488 |
| UBE2E3 | 682401II [symbol] | 0.5278 | 0.2158 | −0.1002 | −0.4922 | −0.4302 | −0.4692 | −0.0453 | 0.1818 | −0.5832 | −1.438 | −0.4452 | 0.6308 | 0.0453 | −1.422 | −0.7132 | 0.4848 |
| UBL5 | 688745II [symbol] | −0.967 | −0.554 | −0.046 | −0.118 | −0.088 | −0.224 | −0.393 | −0.3625 | 0.1825 | 0.372 | 0.108 | 0.41 | 0.288 | 0.52 | 0.336 | 0.151 |
| UBN1 | 687671II [symbol] | 0.1595 | −0.3325 | −0.0035 | −0.3045 | 0.1255 | −0.0365 | 0.3235 | −0.6895 | −0.3955 | −0.1475 | 0.3325 | 0.2865 | −0.0205 | −0.2445 | 0.5145 | 0.1815 |
| UCHL1 | 688791II [symbol] | 0.291 | 0.096 | 1.841 | −0.551 | −0.401 | 0.21 | 0.781 | 1.641 | −0.507 | −1.09 | −0.225 | −0.795 | 0.1005 | −0.253 | 0.918 | 0.565 |
| UCHL3 | 695206II [symbol] | −1.658 | −0.985 | 0.065 | −0.596 | −0.283 | −0.168 | −1.274 | −1.2515 | −0.9345 | −0.925 | 0.01 | −0.862 | −1.2795 | 0.007 | −0.891 | −1.11 |
| UGCG | 682939II [symbol] | 0.2765 | 0.5015 | −0.4795 | 0.1245 | 0.3405 | −0.8485 | −1.763 | −0.3765 | 0.0905 | 3.741 | 0.4865 | 0.0175 | 0.901 | −0.7485 | 0.6075 | −0.1135 |
| UGCGL1 | 686132II [symbol] | −0.535 | −0.51 | −0.981 | −0.176 | −0.485 | −0.377 | −0.199 | −1.039 | −0.8075 | −0.171 | 0.107 | −0.474 | −0.7075 | 0.225 | −0.653 | −0.74 |
| UGT2B7 | 689581II [symbol] | 0.389 | 1.544 | 2.456 | −0.024 | 0.087 | −0.057 | 1.946 | −0.6705 | 7.47 | 4.424 | −0.687 | −0.572 | 0.129 | −0.638 | −0.388 | −0.296 |
| UNC13D | 693680II [symbol] | 0.045 | 0.455 | 0.677 | 0.237 | −0.281 | 1.095 | 3.26 | 0.9015 | 0.653 | 0.551 | 0.285 | 0.038 | −0.4755 | −0.332 | 1.613 | 0.486 |
| VAMP8 | 685078II [symbol] | −0.784 | −0.052 | 0.539 | −0.354 | −0.117 | 0.371 | 0.716 | 0.4105 | 0.5915 | −0.039 | 0.144 | 0.067 | −0.698 | 0.024 | −0.06 | 0.277 |
| VAPA | 686453II [symbol] | 0.451 | 0.175 | −0.877 | 0.107 | 0.156 | −0.496 | −0.677 | 0.223 | −0.4505 | 0.063 | −0.33 | 0.39 | 0.556 | 0.56 | −0.536 | 0.04 |
| VAV1 | 693880II [symbol] | −0.123 | −0.676 | 0.256 | −0.175 | 0.34 | −0.113 | −0.53 | 0.658 | −0.296 | −0.281 | −0.423 | −0.5 | −0.263 | 0.067 | 0.126 | 0.677 |
| VAV3 | 695745II [symbol] | 2.085 | 2.908 | 2.009 | 1.994 | 3.354 | 1.206 | 0.634 | 2.362 | 1.8145 | 2.503 | 2.478 | 3.789 | 3.6055 | 2.111 | 3.427 | 2.187 |
| VCX3 | 688317II [symbol] | 0.501 | 1.65 | −0.548 | −0.55 | −0.567 | 0.011 | 0.23 | 6.411 | 0.5425 | 0.415 | −0.607 | −0.874 | 1.59 | −0.06 | 1.249 | −0.359 |
| VDAC2 | 681387II [symbol] | −0.3335 | −0.0985 | 0.0305 | 0.3935 | −0.1615 | −0.6135 | −1.102 | −0.662 | −0.546 | −0.0475 | 0.2865 | −0.3845 | −0.9655 | 0.1435 | −0.4755 | 0.1255 |
| VEST1 | 685356II [symbol] | 0.2335 | 0.1105 | −0.1045 | −0.1235 | −0.1715 | 0.0905 | −0.0135 | 0.148 | 0.25 | −0.0815 | 0.2435 | −0.1005 | 0.0135 | −0.1875 | −0.0935 | 0.0525 |
| VGLL1 | 763309II [symbol] | −0.8415 | −0.3065 | 1.259 | 0.1545 | 0.2995 | 1.704 | 0.8835 | 0.522 | 0.7155 | 0.0365 | −0.3335 | −0.1415 | −0.7025 | 0.4095 | 0.3445 | −0.0365 |
| VMD2L2 | 696188II [symbol] | 0.082 | 0.492 | 0.593 | 0.421 | 0.268 | 0.852 | 0.212 | 0.818 | 0.8835 | −0.376 | −0.407 | 0.249 | 0.526 | 0.573 | −0.111 | 0.029 |
| VSX1 | 692722II [symbol] | 1.679 | 0.703 | 0.228 | 0.48 | 0.592 | 0.541 | 2.96 | 1.013 | 1.729 | 0.763 | 0.303 | 0.791 | 1.101 | 0.466 | 0.314 | 1.497 |
| WARP | 683039II [symbol] | −0.7765 | −0.5485 | −0.3955 | −0.0135 | −0.4955 | −0.3785 | 0.1125 | −0.0105 | 0.5835 | 0.0105 | −0.2255 | −0.2535 | −0.587 | 0.9525 | 0.0335 | 0.0335 |
| WDR26 | 691481II [symbol] | −0.5025 | 0.0095 | −0.2355 | −0.2945 | 0.9445 | 0.4695 | −0.2705 | −0.3265 | −0.5185 | −0.0385 | 0.4975 | 0.6385 | −0.537 | −0.3165 | 0.0875 | 0.9185 |
| WDR6 | 693627II [symbol] | 0.498 | 0.617 | −0.162 | 0.429 | 0.879 | 0.622 | 0.618 | −0.149 | −0.0425 | 0.283 | −0.357 | 0.376 | 0.45 | 0.252 | 0.714 | 0.033 |
| WHSC1L1 | 695912II [symbol] | 0.592 | −0.033 | −0.689 | 0.327 | 0.486 | 0.195 | 0.796 | −0.188 | −0.295 | 0.617 | 1.098 | 1.123 | 0.3425 | 0.046 | 0.547 | −0.243 |
| WNT3 | 686424II [symbol] | −0.426 | −0.805 | −0.241 | 1.482 | 0.37 | −0.751 | 0.641 | −0.529 | 0.1555 | 0.737 | −0.02 | 0.39 | −0.172 | 0.71 | 0.389 | −0.924 |
| WNT6 | 763326II [symbol] | 0.782 | −0.24 | −0.25 | −0.156 | −0.21 | 0.252 | 0.288 | 2.327 | 0.3455 | −0.074 | −0.436 | −0.399 | −0.1765 | −0.332 | −0.61 | 0.163 |
| WSB2 | 693380II [symbol] | −0.4555 | −0.1415 | 0.4875 | 0.5765 | 0.0885 | 0.5255 | −0.0025 | −0.461 | 0.3685 | −0.3935 | −0.1595 | −0.0385 | −0.171 | 0.2435 | 0.8595 | −0.0845 |
| WWP1 | 686545II [symbol] | −0.8573 | 0.7567 | −0.2473 | 0.8567 | 1.278 | −1.12 | −1.019 | −0.5823 | −0.0288 | 1.342 | 0.7997 | 1.194 | 0.2687 | −0.4463 | 0.4677 | 0.4007 |
| WWP2 | 691487II [symbol] | 0.389 | 0.048 | −0.017 | 0.113 | −0.501 | 0.307 | −0.581 | 0.522 | −0.2845 | 0.095 | 0.231 | −0.312 | −0.327 | 0.203 | −0.039 | 0 |
| XBP1 | 689595II [symbol] | −1.072 | −0.035 | −0.792 | 0.863 | 1.137 | −0.21 | −2.713 | −1.272 | −0.6345 | 1.233 | −0.022 | 0.507 | 0.022 | 1.553 | −0.468 | 0.151 |
| XYLT2 | 692339II [symbol] | −0.095 | −0.256 | −0.406 | 0.141 | 0.225 | 0.668 | 0.166 | 0.6635 | 1.226 | 0.813 | 0.095 | 0.606 | 0.7995 | −0.199 | 0.841 | 0.246 |
| YBX2 | 690818II [symbol] | 0.1375 | 0.0515 | 0.0145 | −1.027 | −1.04 | 0.6275 | 0.3395 | 0.334 | −0.0965 | −0.7035 | −0.0105 | −0.0935 | 0.431 | −0.9875 | −0.0715 | 0.1445 |
| YIF1 | 687006II [symbol] | −0.355 | 0.475 | −0.198 | −0.227 | −0.576 | 0.358 | −0.177 | 0.5015 | −0.432 | 0.014 | 0.04 | −0.043 | 0.025 | 0.317 | −0.33 | 0.277 |
| ZA20D3 | 694225II [symbol] | −0.376 | −0.019 | −1.078 | −0.891 | −0.314 | −1.854 | −1.196 | −1.399 | −1.9335 | −0.814 | −0.632 | −0.353 | −0.791 | −0.545 | −1.426 | −0.264 |
| ZAP1Z8 | 697114II [symbol] | −0.3493 | 0.2927 | 1.839 | 0.0448 | −0.2853 | −0.2403 | −0.1233 | 1.143 | 0.0383 | 0.2147 | −0.3163 | −0.5583 | −0.4458 | −0.0763 | −0.2813 | 0.8537 |
| ZBED3 | 694304II [symbol] | 0.435 | 0.175 | 0.529 | −0.422 | 0.519 | 0.02 | 0.71 | 0.7545 | 0.835 | −0.747 | −0.451 | −0.605 | 0.57 | −0.316 | −0.215 | 0.619 |
| ZBTB4 | 692679II [symbol] | 1.455 | 1.504 | 0.367 | 1.712 | 1.784 | 1.201 | 0.876 | 1.8355 | 1.1585 | 1.155 | 1.097 | 1.269 | 1.355 | 1.576 | 1.216 | 1.245 |
| ZCWCC1 | 694671II [symbol] | 0.049 | 0.573 | 0.684 | 0.383 | 0.631 | 0.451 | 0.783 | 1.6015 | 0.5455 | −0.722 | 0.105 | 0.708 | 0.556 | 0.078 | 0.182 | 0.664 |
| ZCWCC2 | 688993II [symbol] | −0.1665 | −0.1045 | 1.029 | −0.2365 | 0.1945 | 0.9135 | −0.0235 | −0.045 | −0.4805 | −0.1055 | −0.0305 | −0.4985 | 0.7455 | 0.3625 | −0.6295 | 0.7165 |
| ZDHHC13 | 692867II [symbol] | 0.355 | −0.089 | −0.646 | −0.338 | −0.346 | 0.003 | 0.616 | −0.124 | −0.1395 | 0.658 | 0.43 | −1.176 | −0.301 | −0.518 | −0.106 | 0.286 |
| ZDHHC14 | 690817II [symbol] | 0.888 | −0.053 | 0.125 | 0.38 | −0.662 | −0.247 | 1.106 | −0.1365 | 0.117 | 0.158 | −0.716 | −0.499 | −0.31 | −0.691 | −0.362 | −0.194 |
| ZFHX4 | 696157II [symbol] | 1.593 | 0.795 | −0.003 | −0.227 | 0.107 | 0.676 | 0.015 | 2.556 | 1.31 | 0.036 | 1.176 | 0.4772 | 1.1775 | 0.818 | 1.776 | 1.246 |
| ZFYVE21 | 696639II [symbol] | 0.647 | 0.835 | 0.151 | −0.485 | 0.571 | 0.551 | 0.07 | 0.4875 | −0.0275 | 0.019 | 1.098 | 0.648 | 0.668 | 0.061 | 0.634 | 0.216 |
| ZNF133 | 689721II [symbol] | 0.115 | 0.61 | −0.527 | 0.098 | 0.07 | 0.498 | 0.551 | 0.186 | −0.1795 | −0.486 | −0.245 | −0.261 | 0.8345 | −0.565 | 0.107 | −0.325 |
| ABAT | 682378II [symbol] | −1.035 | 0.17 | −2.664 | 0.566 | 3.38 | −1.951 | −1.821 | −2.577 | 1.761 | 1.056 | 1.109 | 2.634 | 2.026 | 1.035 | 1.308 | −0.534 |
| ZNF165 | 688726II [symbol] | −0.1955 | −0.8575 | 0.6975 | 1.12 | −0.1195 | −1.678 | 0.9645 | −0.0665 | −0.0545 | 1.208 | 0.1355 | −0.1375 | −0.881 | 0.3035 | −0.4095 | −0.2185 |
| ZNF24 | 683142II [symbol] | −0.265 | −0.103 | −0.77 | 0.519 | 0.433 | 0.093 | −1.395 | −0.5275 | 0.3775 | 0.736 | −0.711 | 0.181 | 0.0885 | 0.389 | 0.061 | −0.344 |
| ZNF318 | 682803II [symbol] | 0.069 | −1.028 | −0.391 | 0.403 | −0.069 | −0.132 | 0.6 | −0.692 | −0.333 | 0.507 | 0.191 | −0.286 | −0.344 | 0.394 | 0.165 | 0.096 |
| ZNF364 | 690750II [symbol] | −0.44 | −0.021 | −0.184 | −0.244 | 1.175 | −0.566 | −0.127 | −0.703 | −0.3245 | 0.006 | 0.054 | 0.423 | −0.032 | −0.185 | 0.121 | 0.36 |
| ZNF432 | 696694II [symbol] | −0.01 | −0.499 | 0.082 | −0.149 | −0.126 | −0.236 | 0.289 | −0.176 | −0.018 | −0.09 | −0.013 | −0.263 | −0.1725 | −0.585 | −0.199 | −0.025 |
| ZNF462 | 682655II [symbol] | −1.284 | 0.0325 | 0.3455 | 0.2355 | 0.6035 | −0.3985 | −0.2176 | 0 | −0.3955 | 0.3475 | 0.4495 | −0.8205 | −0.483 | 0.1825 | −0.3665 | 0.0085 |
| ZNF505 | 697563II [symbol] | −0.235 | −1.432 | −0.212 | −0.981 | −0.349 | −0.138 | 0.199 | 0.2625 | −0.6395 | −1.155 | −0.379 | −0.76 | −0.2935 | −1.415 | −0.567 | −0.408 |
| ZNF552 | 681805II [symbol] | −1.548 | 0.777 | −1.663 | 0.068 | 1.447 | −0.042 | 0.106 | −0.76 | 1.226 | 1.308 | 1.017 | 0.797 | 0.6195 | 0.042 | 1.178 | −0.059 |
| ZNF577 | 696450II [symbol] | 0.321 | 0.712 | 0.139 | 0.183 | 1.181 | 0.733 | 1.402 | 1.619 | 1.993 | −0.221 | 0.957 | 0.864 | 0.9 | 0.748 | 0.172 | 0.989 |
| ZNF587 | 695267II [symbol] | 0.358 | 1.387 | 0.249 | 1.832 | 1.934 | 1.091 | 1.028 | 0.067 | 1.3975 | 2.257 | 1.306 | 0.396 | 1.119 | 0.74 | 2.358 | 0.357 |
| ZNF598 | 689656II [symbol] | −0.6938 | −0.2228 | 0.2622 | −0.5368 | −0.2118 | −0.2478 | 0.6612 | 0.6202 | 0.0128 | −0.4858 | 0.1722 | 0.1482 | −0.5633 | −0.6408 | −0.2288 | 0.6552 |
| ZNF599 | 697181II [symbol] | 0.253 | 0.331 | −0.297 | 0.771 | 0.651 | 0.457 | 1.152 | 1.8945 | 0.409 | 0.660 | 0.452 | 0.593 | 0.9135 | 1.273 | 0.175 | 0.11 |
| ZNF606 | 683801II [symbol] | −0.3762 | −0.0923 | −0.2222 | 0.1478 | 0.5268 | 0.7288 | 0.8038 | −0.1362 | 0.0083 | 0.5988 | 0.6638 | −0.2602 | −0.2782 | −0.1942 | 0.0478 | −0.0523 |
| ZNF614 | 683555II [symbol] | 0.258 | −0.324 | 0.073 | 0.16 | 0.361 | −0.368 | −0.113 | 0.3545 | 0.007 | −0.055 | 0.416 | −0.393 | 0.0935 | −0.212 | 0.129 | −0.175 |
| ZNF639 | 690004II [symbol] | −0.227 | 0.091 | −1.13 | −0.222 | −0.464 | 0.007 | −1.547 | −0.6165 | −1.2135 | −0.316 | −0.791 | 0.09 | −0.401 | 0.22 | −0.6 | −0.87 |
| ZNF7 | 684715II [symbol] | 1.388 | 1.15 | 0.941 | 1.095 | 2.176 | 0.398 | 1.193 | −0.2835 | 0.8745 | 1.771 | 1.116 | 2.48 | 0.923 | 1.847 | 0.603 | 1.405 |

| UB66 | UB67 | UB71 | UB79 |
|---|---|---|---|
| 1.258 | −2.195 | −2.417 | −1.009 |
| −1.49 | −0.758 | −1.58 | −0.9225 |
| 1.265 | −0.784 | −0.318 | −0.096 |
| −0.312 | 0.4985 | 0.186 | −0.025 |
| −0.997 | 0.1436 | 1.733 | 4.101 |
| 0.663 | −0.3 | 0.281 | 0.539 |
| −1.101 | −0.4495 | −0.114 | −0.0845 |
| 1.649 | −0.1665 | 0.406 | 1.365 |
| 0.182 | −2.157 | −2.106 | 0.423 |
| 0.0325 | −0.435 | −0.504 | −0.566 |
| −0.713 | 1.184 | −0.443 | −0.5105 |
| 0.677 | 0.806 | 1.09 | 1.4095 |
| 0.081 | −0.058 | 0.301 | 0.144 |
| 0.059 | 1.197 | 0.949 | 1.003 |
| 1.412 | −2.586 | 0.971 | −3.3155 |
| 0.087 | 0.344 | 0.184 | −0.5335 |
| 0.233 | −0.302 | −0.507 | 0.876 |
| −0.285 | 0.91 | 0.099 | 0.572 |
| 0.19 | −1.8625 | −3.646 | −3.21 |
| 1.087 | 0.2385 | 0.243 | 0.8425 |
| 0.339 | −0.5255 | −0.513 | −0.3355 |
| −0.34 | −0.2085 | −0.723 | 0.177 |
| 0.1102 | 1.629 | 1.437 | 1.1065 |
| −0.263 | 1.774 | 2.097 | 0.565 |
| 0.425 | −0.2825 | −0.557 | −0.366 |
| 0.6335 | −0.465 | −0.447 | −1.0055 |
| −0.749 | 1.047 | −0.845 | 0.3715 |
| 0.084 | 0.718 | −0.383 | −0.52 |
| −0.126 | 0.508 | 0.054 | −0.296 |
| 1.448 | −0.655 | −0.044 | −0.4085 |
| 1.454 | −0.0355 | 0.547 | −0.1255 |
| 0.246 | −0.8735 | −0.301 | 0.648 |
| 1.278 | −1.1825 | −0.575 | −0.36 |
| −0.0258 | −0.1195 | −0.481 | −0.106 |
| 1.571 | −0.3265 | 0.177 | −1.0425 |
| 0 | 3.542 | 3.412 | 2.4155 |
| −0.401 | 2.032 | 0.443 | −0.6525 |
| −0.2636 | 0.1195 | −0.72 | −2.519 |
| 2.91 | −0.0625 | 0.163 | 2.841 |
| −1.621 | 0.9145 | −0.982 | −2.2475 |
| −0.42 | 0.6335 | −0.316 | −0.0395 |
| −0.9245 | 1.5355 | 0.225 | −1.326 |
| 0.0075 | 3.2705 | 0.45 | 0.528 |
| 3.901 | −0.537 | 0.191 | −0.923 |
| 0.637 | −0.0045 | 1.507 | 1.3285 |
| 0.883 | 1.4 | 0.551 | 0.2565 |
| 0.4 | 1.519 | 0.712 | 0.224 |
| −0.032 | −0.3525 | 0.767 | −0.2155 |
| −0.008 | 0.075 | 0.708 | −0.333 |
| 0.757 | 0.939 | −0.54 | 0.301 |
| 2.02 | 0.137 | 0.349 | 1.413 |
| 2.211 | 0.1095 | 0.573 | 1.5195 |
| −0.197 | 1.413 | 1.132 | −0.3405 |
| −0.363 | 0.324 | −0.931 | 0.2405 |
| −0.1787 | 0.127 | 0.481 | −0.25 |
| 1.183 | −1.8905 | −2.515 | −2.4255 |
| −1.489 | −0.584 | −0.603 | −1.1395 |
| −0.041 | 0.54 | −0.487 | 1.6535 |
| 0.214 | 0.262 | −2.309 | −0.653 |
| −2.568 | −0.647 | 0.23 | −2.489 |
| −0.039 | −0.3445 | −0.395 | 0.042 |
| −0.0803 | −0.437 | 0.31 | −1.277 |
| −0.0445 | 1.475 | 1.048 | 0.599 |
| −1.727 | −0.464 | −0.853 | 0.3445 |
| 1.339 | −1.0525 | −0.801 | −0.207 |
| −0.861 | −0.991 | −0.774 | −0.851 |
| −0.942 | −1.0735 | −0.164 | −0.956 |
| −0.902 | 0.083 | 0.537 | 0.1685 |
| 1.459 | 0.9805 | 0.852 | −1.4675 |
| −0.066 | 1.672 | 0.675 | 2.9835 |
| 0.659 | −0.2805 | 0.367 | 0.414 |
| 0.028 | −1.0495 | −0.428 | −0.7735 |
| 0.6725 | −0.3505 | −0.71 | −0.7465 |
| −0.317 | 1.6915 | 2.679 | −1.3855 |
| −1.107 | 0.121 | −0.384 | 0.993 |
| 0.13 | −0.432 | −0.86 | −0.161 |
| −0.0225 | 0.2445 | −0.241 | 0.7185 |
| 1.587 | −0.877 | −0.478 | 1.0005 |
| −0.459 | 0.5865 | −6.138 | −3.0645 |
| −0.7312 | −0.927 | −0.936 | 1.635 |
| 0.001 | 2.023 | 0.502 | 0.809 |
| 0.355 | −1.8665 | −2.476 | 4.691 |
| −1.022 | −3.348 | −3.083 | 0.6085 |
| −1.475 | 2.2285 | 4.242 | −0.0565 |
| 0.208 | 1.4045 | −1.316 | 0.871 |
| −0.089 | 0.966 | 0.33 | −1.795 |
| −0.717 | 0.2925 | 0.03 | 0.3105 |
| 1.258 | −2.195 | −2.417 | −1.009 |
| 0.5975 | 0.81 | −1.184 | −0.649 |
| −1.239 | −0.387 | −0.987 | −2.025 |
| 0.0095 | 0.451 | −1.301 | −1.024 |
| 0.5085 | −1.227 | −1.921 | −0.512 |
| 1.347 | −0.1035 | −0.251 | −0.337 |
| −3.422 | −0.8526 | −1.303 | −2.201 |
| −0.404 | 1.62 | 0.889 | −0.3725 |
| 0.213 | 0.411 | 0.418 | −1.264 |
| −0.33 | −1.2306 | −1.793 | −1.608 |
| −0.277 | 0.731 | 0.049 | 2.747 |
| 0.279 | 1.434 | 0.534 | 0.005 |
| 0.0735 | −0.0055 | 0.951 | 0.205 |
| −0.1565 | 1.823 | 1.008 | 0.3725 |
| 0.666 | −1.0755 | −0.807 | −0.509 |
| 0.426 | 0.1045 | −0.286 | −2.497 |
| 0.5068 | 1.32 | −0.373 | 0.429 |
| −0.3675 | −1.2405 | −1.13 | −2.7365 |
| 1.191 | −0.8015 | −1.189 | −1.224 |
| −0.549 | −0.775 | −0.502 | −0.3055 |
| −0.3395 | 1.0565 | 1.005 | −0.2755 |
| 1.204 | −1.059 | −1.165 | −1.3515 |
| 3.314 | 1.4345 | 1.71 | 3.514 |
| −0.949 | 0.6965 | 0.339 | −0.259 |
| −0.628 | 0.3045 | 0.041 | −1.114 |
| 0.455 | 1.6675 | 0.636 | 0.707 |
| −0.225 | 0.6865 | 0.141 | −0.4795 |
| 0.878 | 0.3725 | 0.2 | 1.031 |
| −0.278 | −0.1385 | −1.048 | −0.349 |
| −1.183 | −1.063 | 0.315 | −1.096 |
| 1.751 | 1.3175 | 0.343 | 2.555 |
| −0.5485 | 0.7915 | 1.05 | −0.3535 |
| 0.0655 | 1.3725 | 1.598 | 2.2415 |
| −0.3095 | −1.293 | −0.334 | −1.384 |
| 1.038 | 1.406 | 1.874 | 1.6695 |
| −0.1352 | −0.4375 | −0.028 | 1.381 |
| 1.361 | 1.959 | −0.323 | 0.158 |
| −1.183 | 2.105 | 2.321 | 0.816 |
| 0.577 | −0.685 | −0.078 | 0.788 |
| −1.693 | −0.9595 | −0.932 | 1.1 |
| 0.2685 | 2.385 | 0.991 | 1.7925 |
| 2.217 | 0.584 | −2.274 | 0.1105 |
| 1.456 | 1.9405 | 0.594 | 1 |
| −0.7735 | −0.682 | 0.515 | 1.4945 |
| 1.833 | 1.363 | 0.438 | 0.127 |
| −0.452 | −0.072 | 1.75 | 1.684 |
| −0.056 | −1.374 | −0.164 | −2.1765 |
| 0.3075 | −0.7555 | 0.355 | 0.7635 |
| 0.139 | −0.029 | 0.218 | −0.836 |
| −0.727 | −0.302 | 0.62 | 1.0825 |
| −1.139 | −0.6455 | −1.273 | −1.326 |
| 0.077 | −1.275 | −0.43 | −1.0395 |
| 0.739 | 0.92 | 0.101 | 0.536 |
| 0.376 | 0.342 | 0.549 | 1.7015 |
| −0.779 | −0.5425 | −1.184 | −1.498 |
| −0.367 | 0.132 | −0.469 | −1.35 |
| −0.944 | 0.25 | 0.971 | 0.6875 |
| 0.077 | 0.3895 | −0.139 | −0.574 |
| −0.116 | −0.4255 | −1.607 | −1.9085 |
| −0.326 | −0.9225 | −0.724 | 0.073 |
| 0.912 | −1.07 | −0.167 | −0.0015 |
| −0.0195 | 0.663 | 1.216 | 0.259 |
| 1.034 | 1.226 | 0.054 | 0.384 |
| 0.428 | −1.88 | −1.85 | −1.185 |
| −0.6415 | 0.277 | 1.573 | −1.052 |
| −0.498 | −0.131 | 0.055 | 0.069 |
| −0.5895 | 0.2945 | 1.113 | −1.396 |
| 1.957 | −0.9075 | −0.657 | 2.262 |
| 1.161 | −0.336 | −0.934 | −0.642 |
| 0.6075 | −0.7015 | −1.855 | −1.0265 |
| −0.381 | 0.0835 | 0.262 | 0.175 |
| −0.301 | 0.329 | −0.2 | 0.0965 |
| −0.4175 | 0.849 | −0.747 | −0.451 |
| −0.72 | −0.6345 | 3.082 | −0.5465 |
| −0.69 | −0.4865 | −1.205 | 1.895 |
| 0.665 | 0.544 | −1.426 | −0.758 |
| 0.282 | 0.7705 | 0.63 | −0.211 |
| 0.6957 | −0.013 | 0.302 | 2.853 |
| −1.937 | 2.012 | 1.326 | 1.179 |
| −0.795 | 1.244 | 0.343 | 2.1755 |
| 4.713 | 1.77 | 0.171 | 0.592 |
| 1.261 | 2.3235 | 1.016 | 0.763 |
| −0.519 | 1.0175 | 0.746 | −0.0465 |
| 0.403 | 1.882 | 0.392 | −0.213 |
| 0.6257 | 0.312 | 0.121 | 0.313 |
| 1.582 | −0.296 | 0.767 | −0.2115 |
| −1.436 | 1.103 | 1.698 | 0.459 |
| 1.258 | −2.195 | −2.417 | −1.009 |
| 1.429 | 0 | −0.476 | −0.8535 |
| −0.142 | 0.333 | 0.467 | 0.582 |
| −1.831 | −1.397 | −1.56 | −1.416 |
| −0.243 | −0.865 | 0.286 | −0.8305 |
| 0.981 | −1.623 | −2.596 | 0.626 |
| −0.7762 | 0.5255 | 0.161 | −1.2745 |
| 0.8405 | 0.8165 | 0.556 | −0.345 |
| −0.3235 | 0.5325 | −0.516 | −0.3915 |
| 0.167 | 1.79 | 1.393 | 0.807 |
| −3.009 | −0.4945 | −1.376 | −1.334 |
| −0.232 | 1.018 | 1.379 | 1.387 |
| −0.314 | 0.5875 | −0.031 | −1.067 |
| 0.7755 | 2.253 | 0.248 | 0.3655 |
| −2.851 | −1.9635 | −0.883 | −2.725 |
| −2.918 | −2.0965 | −1.851 | −2.795 |
| −3.108 | −0.3275 | 0.045 | −1.9595 |
| −3.466 | −1.157 | −1.253 | −1.2285 |
| −0.623 | 0.9805 | −0.301 | 1.6545 |
| 1.163 | 0.907 | 0.962 | 0.999 |
| −1.137 | 2.571 | −0.224 | 0.346 |
| −1.087 | −0.689 | −0.848 | −0.4865 |
| −0.152 | 1.1865 | 0.949 | −0.3375 |
| −0.921 | 1.1575 | 0.435 | 0.4235 |
| −0.0035 | −1.604 | −0.956 | −2.315 |
| −0.0685 | −2.0075 | −1.438 | −3.039 |
| −0.6165 | 1.662 | 0.172 | −0.011 |
| −0.8515 | −0.3005 | 0.423 | 0.542 |
| −1.464 | −0.6685 | −0.082 | −1.491 |
| −1.421 | −0.57 | −0.873 | −2.3655 |
| −0.254 | −0.12 | 0.169 | −2.413 |
| −0.364 | 0.828 | 0.407 | 1.023 |
| −0.399 | −0.0025 | −0.276 | −0.0165 |
| −0.306 | −1.072 | −0.486 | −0.513 |
| −2.727 | −3.1285 | −2.381 | −1.2175 |
| 0.364 | 0.0845 | 0.406 | −0.1355 |
| −0.464 | −0.657 | −1.475 | −1.132 |
| 0.238 | −0.332 | 0.34 | −0.2346 |
| −1.621 | 1.7295 | 0.546 | 3.096 |
| −0.3045 | 1.0635 | −0.37 | 3.116 |
| 0.04 | −1.015 | −1.228 | −1.1525 |
| −0.872 | 0.642 | −1.036 | −1.5495 |
| 0.1065 | −0.913 | 1.782 | −0.46 |
| −0.0175 | −0.383 | −0.089 | 0.5265 |
| 1.945 | 2.8015 | 1.544 | 2.6545 |
| −1.524 | 0.602 | −1.29 | −1.1545 |
| −0.8735 | 0.027 | −1.251 | −0.01 |
| −3.334 | −1.5585 | 0.514 | 1.554 |
| 1.023 | 1.062 | 0.132 | −0.5355 |
| −0.4035 | 0.139 | 0.408 | 0.4325 |
| 0.384 | −0.0925 | 0.505 | −0.1265 |
| 0.793 | 0.9865 | 0.682 | 0.7365 |
| 0.609 | −0.3775 | 0.186 | 0.417 |
| 0.2076 | 0.2285 | −0.195 | −1.1965 |
| 0.302 | 0.882 | 0.67 | 0.7495 |
| 0.0941 | 4.8515 | 2.996 | 3.2155 |
| 0.958 | 0.2615 | 1.746 | 2.5585 |
| −1.968 | −0.437 | 0.298 | −1.3425 |
| −0.701 | −1.3765 | −1.081 | −2.2975 |
| −0.898 | 0.6165 | −0.209 | −0.882 |
| −0.576 | 1.8525 | 2.96 | 1.53 |
| −2.106 | 2.1845 | 0.138 | 2.4485 |
| −3.392 | 2.701 | 1.543 | 3.3105 |
| 0.395 | 0.2735 | −0.228 | 0.3885 |
| 3.9 | −2.72 | −3.005 | −2.5685 |
| −0.566 | −0.0145 | −0.116 | −0.424 |
| −0.204 | −0.8545 | −0.704 | 0.496 |
| −0.02 | 0.211 | −0.79 | −0.7755 |
| 1.34 | 1.176 | −2.197 | 1.535 |
| −0.202 | −1.27 | −0.259 | −0.883 |
| 0.08 | −0.2785 | 0.089 | −0.455 |
| 1.936 | −0.829 | −0.258 | −1.2165 |
| 0.095 | −0.5125 | −0.638 | −0.0615 |
| −0.2075 | 0.3275 | −0.149 | 0.4815 |
| 0.3435 | −0.451 | 0.124 | −0.294 |
| −0.591 | 0.039 | −1.195 | −0.806 |
| −0.092 | 0.139 | −1.183 | 3.671 |
| −0.462 | −1.058 | −0.779 | −0.099 |
| −0.044 | −0.224 | −0.378 | −0.6265 |
| −0.8995 | 1.207 | −0.813 | −0.6985 |
| 0.467 | 0.287 | −1.328 | −1.3715 |
| 0.719 | 1.232 | 0.107 | −0.8745 |
| −2.762 | −1.61 | 0.074 | −1.818 |
| −0.679 | −0.067 | 0.348 | 0.2415 |
| −0.0975 | −0.125 | 0.76 | 0.659 |
| −0.088 | −0.2345 | −0.122 | −0.2375 |
| 0.0785 | −0.08 | −0.322 | 0.4685 |
| 1.258 | −2.165 | −2.417 | −1.009 |
| −0.1075 | 0.1415 | 0.117 | 0.099 |
| 0.32 | −0.1655 | 0.276 | 1.5745 |
| 0.375 | −0.879 | −0.885 | −0.941 |
| −0.343 | −0.7915 | −0.65 | −0.945 |
| 0.1085 | −0.127 | −0.037 | −0.0165 |
| 0.0565 | −1.397 | −0.141 | 3.765 |
| −1.345 | 0.0195 | 0.406 | 0.3675 |
| 0.1105 | 0.1705 | −0.462 | −0.63 |
| 0.22 | 0.168 | −0.434 | −0.373 |
| −0.913 | −0.3035 | 0.272 | 2.414 |
| 0.3345 | 1.497 | 0.064 | 1.863 |
| −0.993 | −1.7815 | 2.944 | −2.5135 |
| 1.06 | −0.1395 | 0.909 | 1.77 |
| 1.301 | 0.058 | 0.136 | 0.8715 |
| −1.24 | −1.4905 | −2.4 | −1.48 |
| −0.043 | 1.0625 | 1.494 | 3.027 |
| −0.361 | −0.633 | −0.305 | −0.254 |
| 1.149 | −0.0155 | 0.02 | −0.129 |
| 0.5885 | −0.6405 | −0.736 | −1.3725 |
| −0.4495 | −1.2285 | 0.031 | −0.7585 |
| −1.866 | −1.7125 | −0.722 | −0.3525 |
| −0.152 | 0.0595 | 0.276 | 0.5095 |
| 0.372 | 0.3625 | 0.831 | 0.574 |
| −0.3165 | 0.0405 | 1.087 | −1.129 |
| −1.802 | 3.0385 | 4.269 | 2.2935 |
| 0.6285 | 0.6915 | 1.558 | −1.153 |
| −0.4667 | 4.266 | 3.839 | −2.1665 |
| −0.571 | 0.363 | −0.506 | 0.8255 |
| 0.36 | 0.497 | 0.429 | −0.457 |
| 0.8485 | −1.7985 | −1.104 | −2.7145 |
| −0.114 | −0.1185 | −0.296 | −0.589 |
| −0.577 | 2.3735 | 1.6 | 1.6125 |
| −3.524 | −0.668 | −0.876 | −2.4285 |
| 0.6085 | 3.4205 | 2.738 | 1.37 |
| 0.054 | −0.111 | −0.946 | −0.4385 |
| 1.517 | 3.6885 | 1.7 | 2.5665 |
| 2.521 | 5.5665 | 4.36 | 5.5545 |
| −0.6105 | 0.2905 | 0.361 | −0.163 |
| 0.9885 | −0.78 | −1.917 | 1.54 |
| 0.519 | 0.422 | 3.036 | 1.6335 |
| −0.195 | −0.1385 | 0.723 | −0.067 |
| 0.7075 | 2.2215 | 0.843 | 1.1445 |
| 0.494 | −0.0935 | 0.552 | 2.24 |
| 1.3 | 0.084 | 0.425 | 0.3815 |
| 3.03 | 2.1005 | −0.022 | 2.3625 |
| 1.18 | −3.612 | −3.176 | −1.856 |
| 0.7755 | −0.873 | −0.568 | −0.536 |
| 2.313 | 0.033 | 0.267 | 2.293 |
| 3.171 | 0.1115 | 0.035 | 3.0525 |
| 0.617 | −0.4355 | −0.288 | 0.794 |
| −1.054 | −0.8135 | −0.643 | −0.6945 |
| 0.4815 | 0.8605 | 0.135 | 0.417 |
| −0.4575 | 1.629 | 2.309 | 0.0435 |
| −0.755 | 0.134 | 0.421 | −0.764 |
| −1.408 | −0.431 | −0.679 | −1.213 |
| −0.457 | −0.274 | −0.744 | −0.8945 |
| 0.1695 | 0.232 | −0.447 | 1.5075 |
| −0.406 | −0.8355 | −0.377 | 0.023 |
| −0.7805 | −0.8835 | −0.263 | −0.2755 |
| −0.412 | 0.66 | −0.623 | −0.9925 |
| 1.626 | 3.4315 | 1.017 | 1.746 |
| −0.437 | 1.326 | 0.755 | 2.141 |
| 0.31 | −0.943 | −1.472 | −1.5895 |
| 2.3 | −1.1825 | −1.553 | −1.315 |
| 1.112 | 0.2385 | −0.622 | −0.0625 |
| −0.003 | 2.26 | 1.118 | 2.027 |
| 0.288 | 1.262 | −0.328 | 0.571 |
| 1.046 | −0.036 | −0.234 | 0.0765 |
| 0.45 | −0.5915 | 0.118 | −0.1505 |
| 0.72 | 3.3175 | 0.68 | 0.656 |
| −0.8905 | 0.3025 | −0.794 | −0.785 |
| −0.6625 | −0.652 | −0.645 | −1.1985 |
| −0.542 | 1.2025 | −0.111 | 0.0965 |
| 0.194 | 0.149 | −0.234 | 0.3625 |
| −0.436 | −0.8835 | −0.527 | −0.305 |
| 0.2015 | −0.311 | 0.202 | −0.1965 |
| −0.036 | 0.8115 | −0.066 | 0.4745 |
| 0.273 | −0.8705 | −1.132 | −1.035 |
| −0.6257 | 0.706 | 0.119 | 1.9065 |
| 0.096 | −5.587 | −4.343 | −4.6435 |
| 2.254 | −4.279 | −2.188 | 2.7735 |
| 0.033 | 0.5705 | 0.836 | 3.1925 |
| 2.732 | 0.4465 | −0.089 | 0.3945 |
| 0.697 | −2.23 | −2.726 | 0.926 |
| −0.0205 | 0.5885 | 1.324 | −0.7845 |
| 3.531 | 2.487 | −0.575 | 4.2555 |
| 1.258 | −2.195 | −2.417 | −1.009 |
| 0.011 | −0.1015 | −0.365 | −0.2375 |
| 0.53 | −0.052 | 0.296 | −0.2565 |
| −0.362 | 0.077 | −0.8 | −0.657 |
| −0.1905 | −0.41 | 0.256 | −0.405 |
| 1.059 | 0.3665 | 0.568 | 1.4715 |
| 0.0005 | −0.3235 | −0.156 | 0.7935 |
| −0.8157 | −0.261 | −0.471 | −0.03 |
| 0.023 | 0.0865 | 0.67 | −0.266 |
| 0.2435 | 0.5205 | −1.574 | 3.0035 |
| −0.361 | 1.007 | −0.051 | 0.1825 |
| −0.1285 | −0.547 | 1.549 | 1.3415 |
| −1.337 | 0.1675 | 0.073 | 0.5315 |
| −1.186 | 0.014 | −0.488 | 1.22 |
| 2.108 | −1.563 | −2.78 | 3.224 |
| −0.048 | 0.857 | −0.4 | −0.244 |
| −1.805 | −0.884 | −0.111 | −1.772 |
| 0.193 | 0.769 | 0.328 | −0.1145 |
| 0.617 | 0.0935 | −1.093 | −0.27 |
| −0.788 | 0.274 | −0.44 | −0.547 |
| 0.1137 | 0.385 | 0.207 | 0.2005 |
| 3.148 | 2.7105 | 1.277 | 2.262 |
| 1.186 | 1.3185 | −0.781 | 2.707 |
| 1.121 | 0.105 | −0.618 | 0.6415 |
| −0.674 | −0.582 | −0.74 | 0.003 |
| 0.686 | 1.909 | 2.446 | 1.319 |
| −0.596 | −0.4665 | −0.73 | −0.196 |
| −0.63 | −0.173 | −0.223 | 0.322 |
| 0.4235 | −1.7485 | −1.033 | −1.6895 |
| −0.285 | −0.4285 | 0.803 | −0.0485 |
| 0.665 | 0.981 | 0.176 | 0.5875 |
| −0.853 | 4.7405 | 3.037 | 3.714 |
| −0.008 | −0.0805 | 0.441 | 0.508 |
| 0.237 | −0.5 | 0.481 | 0.9265 |
| −0.8225 | 0.16 | 0.621 | 0.8115 |
| 0.228 | 0.044 | 0.151 | 1.2785 |
| 0.8535 | 1.4705 | −0.444 | 1.491 |
| 0.351 | 0.7235 | −1.656 | 0.561 |
| 0.4075 | −1.0285 | −1.445 | 0.2645 |
| −0.326 | −1.8955 | −2.133 | −1.351 |
| 1.489 | −1.2625 | −0.658 | −1.427 |
| 0.317 | 0.074 | −0.454 | −0.5385 |
| 0.0723 | 2.5855 | 1.048 | 1.6505 |
| 0.463 | 0.6945 | 0.291 | 0.28 |
| −0.286 | −0.2555 | −0.167 | 1.296 |
| 0.97 | 0.0005 | 0.041 | 0.4816 |
| −0.823 | −0.924 | 2.454 | −0.707 |
| 1.04 | 0.5805 | −0.145 | 0.457 |
| 0.1495 | −0.372 | −0.391 | −0.1545 |
| 0.187 | −0.7765 | 0.284 | −2.269 |
| −0.939 | −0.4045 | 0.446 | −1.305 |
| 2.718 | 0.3295 | −0.022 | 0.376 |
| 0.219 | 3.606 | −0.129 | −0.4445 |
| −0.431 | −0.344 | −0.901 | 0.1985 |
| −1.743 | −0.9195 | −2.003 | −2.1255 |
| 3.41 | −4.2305 | −4.225 | 1.984 |
| −1.506 | 1.8 | 1.7 | −1.3745 |
| 0.089 | 0.787 | 0.577 | 1.8715 |
| −0.7692 | −0.7985 | −0.41 | −1.836 |
| 0.605 | 0.1055 | −0.178 | −0.157 |
| 0.389 | −1.1115 | −0.067 | −0.4795 |
| 0.4925 | 1.291 | 1.46 | 0.4245 |
| 1.016 | −0.2855 | −0.944 | −0.584 |
| −0.1545 | 2.534 | 2.661 | 1.03 |
| 1.065 | 1.682 | 0.003 | 0.159 |
| 0.183 | −0.5665 | 0.626 | −1.507 |
| 0.004 | 2.016 | 0.741 | 0.188 |
| −0.134 | −0.3395 | 0.082 | −0.377 |
| 3.363 | 3.1745 | 0.252 | 2.73 |
| 2.735 | 1.4945 | 1.555 | 3.127 |
| −0.14 | −0.0325 | −1.016 | −1.1245 |
| 1.177 | 0.379 | 0.103 | −0.087 |
| −3.216 | −1.122 | −1.598 | −2.0735 |
| −0.874 | −0.95 | −1.045 | −0.5125 |
| 1.827 | −0.206 | −1.004 | 1.9315 |
| −0.125 | 0.0325 | −0.39 | −0.845 |
| 1.486 | 0.049 | −1.091 | 1.506 |
| 0.29 | −2.118 | −1.252 | 0.638 |
| −1.049 | −2.305 | −1.048 | −2.2145 |
| −1.663 | −0.0635 | −0.418 | −0.7835 |
| −0.37 | −1.0095 | −0.61 | −1.177 |
| 1.072 | −1.232 | −0.267 | 2.021 |
| −0.459 | 2.8805 | 3.832 | 0.714 |
| 0.5005 | 0.903 | 0.606 | 0.311 |
| 0.1365 | −0.952 | 0.164 | 0.7875 |
| 0.2135 | 0.0965 | −0.066 | −0.8175 |
| −0.5405 | 0.659 | 0.16 | 0.6445 |
| 1.258 | −2.195 | −2.417 | −1.009 |
| −0.1105 | −0.684 | −0.567 | 0.261 |
| −0.2485 | −0.083 | 0.3 | 1.118 |
| −0.1655 | 0.788 | −0.321 | −0.412 |
| 0.353 | −0.154 | 0.788 | 0.459 |
| −0.393 | 0.6605 | 0.177 | −0.995 |
| 0.6925 | 1.521 | −0.242 | 0.3175 |
| −0.956 | −0.701 | −0.54 | 0.809 |
| 0.281 | −0.237 | −0.127 | 0.9805 |
| −0.788 | 0.283 | 1.671 | −1.5575 |
| 0.654 | −0.264 | −1.174 | 1.3395 |
| 0.1165 | 1.6435 | 1.832 | 1.087 |
| 0.178 | −1.7625 | −1.344 | 0.3855 |
| −0.7413 | −0.1455 | 0.13 | 0.7565 |
| 0.783 | 0.478 | 0.62 | 3.377 |
| −0.519 | 1.4335 | −0.077 | 0.8025 |
| −0.125 | −0.505 | 0.737 | −1.6875 |
| −0.162 | 0.761 | 0.045 | −1.3295 |
| −0.2828 | −0.2715 | 0.414 | 0.621 |
| −0.191 | 0.313 | 0.497 | 0.9105 |
| −1.486 | −0.127 | −1.14 | −1.3275 |
| −0.422 | 1.8345 | −1.245 | −0.5285 |
| −1.27 | 0.6325 | −0.311 | 2.0815 |
| −0.142 | 0.8035 | 0.287 | −0.13 |
| −0.194 | −0.331 | 1.194 | 1.256 |
| 0.232 | −2.447 | −1.959 | −0.893 |
| −0.474 | 0.4995 | 0.615 | −1.42 |
| −0.82 | −0.7525 | −0.549 | −0.8585 |
| 1.523 | 1.0405 | −0.62 | 1.6135 |
| −0.663 | −0.5215 | 0.112 | −0.5505 |
| 0.178 | 0.2615 | 0.681 | 0.4705 |
| 0.724 | 1.6955 | 0.367 | 0.093 |
| 0.9095 | −1.145 | −1.157 | −1.7725 |
| −0.0665 | −0.218 | −0.133 | −0.894 |
| 0.4675 | −0.072 | −0.286 | −0.4725 |
| −0.843 | 0.321 | 0.967 | −0.7215 |
| 0.208 | −0.102 | 1.15 | 0.5075 |
| −0.1285 | −0.471 | −0.444 | −1.663 |
| 2.003 | −0.989 | −0.703 | −1.725 |
| 0.252 | 1.1985 | −0.583 | 0.069 |
| −1.537 | −0.303 | −0.174 | −1.7355 |
| −0.14 | −0.4285 | −0.365 | 0.8145 |
| −0.41 | 0.041 | −0.252 | −0.0495 |
| 0.671 | −0.2025 | 0.153 | −0.4175 |
| −1.347 | −1.228 | −0.929 | −2.37 |
| 2.231 | 2.981 | 1.378 | 2.4275 |
| −1.607 | −0.0935 | 0.635 | −0.2905 |
| 0.0385 | 0.7195 | 0.139 | 2.534 |
| 0.432 | −0.149 | −0.025 | 0.4995 |
| 0.0605 | 0.2235 | 0.548 | 0.4185 |
| 0.228 | 0.5295 | 0.301 | 2.9505 |
| 0.59 | 0.25 | −0.454 | 0.76 |
| 2.206 | −0.4335 | −0.431 | −1.23 |
| 3.527 | −2.553 | −3.169 | 3.557 |
| 1.702 | −0.966 | −0.492 | −0.8135 |
| 1.716 | −0.909 | −0.357 | −0.8245 |
| 1.186 | −0.845 | −0.228 | −0.6295 |
| −0.695 | 0.3805 | −0.407 | −0.098 |
| 0.358 | −0.0515 | −0.018 | −0.223 |
| 1.671 | 1.447 | −1.666 | −1.119 |
| 0.076 | 0.4945 | −0.16 | −1.669 |
| 0.2605 | 0.397 | 0.297 | −0.662 |
| 0.6837 | −0.6425 | −0.972 | −0.6905 |
| −0.381 | −0.897 | 1.307 | 0.19 |
| −0.57 | −0.5305 | 2.258 | −0.4535 |
| 1.492 | 0.04 | −1.618 | 1.7205 |
| −0.69 | −0.8025 | −1.432 | −1.235 |
| −1.114 | −0.1985 | −0.562 | −0.601 |
| −0.6585 | 0.9565 | −0.441 | 3.8875 |
| 0.0225 | −4.5635 | −4.29 | −3.513 |
| −0.611 | −0.133 | 0.154 | −0.53 |
| 0.3275 | 0.9065 | −0.738 | 0.3805 |
| −1.072 | −0.812 | −0.177 | 0.001 |
| 0.577 | −0.525 | −1.202 | −1.237 |
| −0.984 | 2.112 | 1.374 | −0.016 |
| −0.2385 | 0.57 | −0.075 | −1.03 |
| 0.359 | −0.1105 | 0 | −0.638 |
| −1.037 | −0.5005 | −0.508 | −1.2295 |
| −0.53 | −1.034 | −1.77 | −0.922 |
| 0.204 | 0.2945 | −0.25 | 0.4155 |
| 2.689 | 2.1635 | 0.961 | 0.2675 |
| −0.535 | 0.4705 | −1.069 | −1.3775 |
| 0.0025 | −0.2135 | −0.162 | 0.585 |
| −0.236 | −0.5965 | −0.533 | −1 |
| 0.098 | −0.3985 | −0.647 | −0.669 |
| −0.3205 | −0.9625 | 0.008 | −1.5555 |
| 0.512 | 0.762 | 0.329 | −0.92 |
| 1.258 | −2.195 | −2.417 | −1.009 |
| −0.159 | −0.0935 | −0.366 | 0.0775 |
| −0.176 | −0.18 | 0.199 | −0.5095 |
| 0.044 | −0.242 | −0.839 | −1.3275 |
| 0.0125 | 0.6385 | 0.612 | −0.687 |
| 0.1365 | 0.1145 | −0.206 | −0.0855 |
| 0.3515 | −0.4215 | 0.353 | 0.4575 |
| −1.23 | 0.6445 | 1.222 | 1.3035 |
| −0.649 | 4.5325 | −0.392 | −0.304 |
| 1.709 | 2.7645 | 2.565 | 1.774 |
| 0.034 | 0.9895 | 0.925 | 0.275 |
| 0.3998 | 1.3225 | 0.214 | 0.2365 |
| 0.975 | 2.561 | 0.352 | 1.8775 |
| 1.022 | −0.052 | −0.339 | −0.266 |
| −0.507 | −0.3225 | 0.322 | 2.332 |
| −0.0445 | −0.369 | −1.545 | −2.353 |
| 0.144 | 1.412 | 1.862 | 2.4745 |
| 1.449 | 1.6985 | 2.073 | 1.783 |
| −0.0505 | −0.2825 | 0.842 | 2.148 |
| 2.5 | 2.6355 | 4.269 | 2.4885 |
| 0.981 | 0.988 | 2.227 | 2.311 |
| 0.0065 | 1.5165 | 2.321 | 1.6265 |
| 0.386 | −1.18 | 0.598 | 0.409 |
| 1.387 | 1.21 | −0.559 | −0.3525 |
| −0.1165 | −0.112 | −0.571 | 0.0805 |
| −0.128 | 0.3015 | −0.739 | −0.2395 |
| 1.075 | −0.6275 | −0.287 | 0.4105 |
| 0.726 | 0.608 | 0.533 | 0.205 |
| −0.447 | 1.1525 | 1.963 | −0.6745 |
| 0.1415 | 0.3015 | 0.195 | −0.8805 |
| 3.198 | 3.8165 | 0.625 | 1.187 |
| 0.871 | −0.3445 | −1.235 | −0.3215 |
| −0.444 | −0.078 | −0.052 | 0.0025 |
| 0.829 | 2.8695 | −0.014 | 0.28 |
| −0.1045 | 0.3065 | 0.258 | 1.8485 |
| −0.6255 | −0.1055 | −0.055 | −0.068 |
| −0.1545 | −0.694 | −0.474 | −0.3695 |
| 0.031 | 2.487 | 2.753 | 2.8755 |
| 2.046 | 0.732 | 1.355 | 3.241 |
| 0.0855 | −0.8785 | −0.64 | 0.3195 |
| 1.271 | 2.3555 | 1.02 | 1.02 |
| 0.139 | 0.0685 | 0.205 | 1.213 |
| 1.31 | 0.112 | −0.151 | 0.331 |
| 0.407 | 0.1765 | 1.185 | 0.283 |
| 0.526 | 1.1625 | −0.947 | 2.2165 |
| −0.662 | 0.3355 | 0.879 | −0.8795 |
| 0.5825 | 0.409 | −0.046 | 0.0925 |
| 0.9 | 0.4315 | 0.633 | 0.454 |
| −0.017 | 0.0325 | 0.613 | 0.6255 |
| 0.3055 | −0.4245 | −0.857 | −0.7945 |
| 1.761 | 2.0545 | −0.316 | 0.9995 |
| 0.8855 | 1.4385 | 3.093 | 1.4235 |
| 1.039 | −0.803 | −0.269 | 0.4745 |
| −0.096 | 0.1025 | −0.016 | −0.3115 |
| −0.298 | −0.1335 | −0.261 | −1.0825 |
| −0.3145 | −1.658 | −1.896 | −2.3585 |
| −0.961 | 0.325 | 0.45 | −0.5805 |
| 0.1907 | −0.2915 | 0.78 | 1.364 |
| −0.5505 | 0.0555 | 0.758 | 0.7425 |
| −0.723 | 0.665 | 0.171 | −1.14 |
| −0.0505 | −0.0215 | −0.419 | 1.066 |
| −0.2865 | 0.132 | 0.024 | 0.3955 |
| −0.934 | 0.2825 | 0.402 | 0.3435 |
| 0.618 | 2.4105 | 1.053 | 1.0115 |
| 0.7325 | 1.3465 | 0.013 | −0.3305 |
| −0.5665 | −0.0705 | 0.474 | 1.115 |
| 1.499 | 0.5785 | 0.707 | 0.341 |
| −1.569 | 0.58 | 0.671 | 0.851 |
| −0.4868 | −1.812 | −1.541 | 2.0035 |
| −0.435 | 0.0815 | 0.057 | −0.6605 |
| −0.1715 | −0.0135 | 0.139 | 0.3075 |
| 0.626 | 0.993 | 0.468 | 3.0465 |
| 0.986 | −0.643 | −0.316 | −0.061 |
| −0.502 | −0.0655 | 0.256 | 0.2105 |
| −0.275 | −0.062 | −0.042 | 0.9035 |
| −0.464 | 2.8445 | 1.853 | 0.5285 |
| −0.133 | 3.849 | 1.878 | 0.8505 |
| −0.8157 | 2.9415 | 4.429 | −0.6495 |
| 0.03 | 0.51 | 3.39 | 0.09 |
| −1.38 | 2.3225 | 4.097 | 0.179 |
| −2.036 | 0.104 | −0.236 | −2.1005 |
| −0.2175 | −0.759 | −1.529 | −2.13 |
| 2.294 | −1.326 | 1.69 | 1.8555 |
| −0.657 | −0.6555 | 2.257 | −0.322 |
| −0.197 | 0.1725 | 0.083 | 0.293 |
| −0.487 | 0.3015 | −0.352 | −0.1935 |
| −1.422 | −1.289 | 1.736 | −0.366 |
| 1.258 | −2.195 | −2.417 | −1.009 |
| 0.563 | −2.956 | −3.87 | −0.348 |
| 0.093 | 4.7295 | 2.699 | −1.515 |
| 0.008 | 2.1405 | 3.975 | 2.039 |
| 0.0635 | 0.261 | −0.025 | 0.5405 |
| −0.3995 | −0.3195 | 0.035 | 0.544 |
| 0.3635 | −0.8765 | −0.98 | −0.1835 |
| −2.289 | −1.8425 | −2.52 | −0.903 |
| −0.073 | −0.087 | −0.298 | −0.472 |
| 0.0245 | 1.2265 | 2.086 | 1.8925 |
| −0.3573 | −0.863 | 0.384 | −1.8695 |
| −1.686 | 2.666 | −1.117 | 0.822 |
| 0.4805 | −1.852 | −3.066 | −2.9065 |
| 0.684 | 0.019 | 0.392 | −1.0545 |
| 0.583 | −1.0995 | −0.706 | 0.256 |
| 0.3245 | 0.6075 | 0.447 | 3.0075 |
| −1.129 | 0.944 | −0.545 | 1.865 |
| 0.085 | −0.385 | −0.046 | −0.3695 |
| −0.6785 | 0.1345 | −0.172 | −0.184 |
| −2.541 | −0.9975 | −0.934 | −1.949 |
| 0.333 | 0.0915 | −0.2 | −0.4125 |
| −0.503 | −0.2495 | −0.564 | −1.283 |
| −0.4285 | −0.768 | −0.69 | −0.5655 |
| 2.482 | 0.888 | 0.253 | 0.182 |
| 0.036 | −3.334 | −1.825 | −2.441 |
| 2.676 | 0.912 | −1.122 | 2.8355 |
| −0.2845 | 0.1275 | 0.216 | 1.0495 |
| 1.096 | −1.9835 | −0.92 | 1.7415 |
| 2.108 | −0.478 | −0.217 | 0.1575 |
| 1.465 | −1.0465 | −1.58 | 1.9735 |
| 0.6025 | 1.3315 | −3.584 | −3.1905 |
| 0.071 | 0.0585 | −0.438 | −0.022 |
| 0.0455 | −0.938 | −1.324 | 2.483 |
| 0.348 | 0.541 | 0.356 | 1.1935 |
| 0.836 | −0.523 | −2.5 | −2.625 |
| −2.092 | −1.5505 | −1.25 | −2.2795 |
| 1.184 | 0.1935 | −0.992 | 2.281 |
| 0.107 | −0.153 | −1.082 | 0.0355 |
| 0.463 | 0.0205 | 0.278 | 0.492 |
| 0.1295 | 0.513 | −0.024 | −0.1725 |
| −1.282 | −1.3755 | −1.091 | −1.338 |
| 0.8525 | −0.616 | −0.595 | 0.258 |
| 0.7065 | −0.107 | 0.189 | 0.4985 |
| 0.829 | −0.072 | −0.115 | −0.495 |
| 0.44 | −0.598 | −0.721 | −0.709 |
| 1.039 | 0.4585 | −0.597 | −0.535 |
| 2.739 | 0.888 | 0.651 | 1.338 |
| −0.6915 | −0.695 | 0.762 | −0.07 |
| 0.642 | 1.0335 | −1.075 | −2.235 |
| −0.3255 | 0.0665 | −0.432 | 0.3705 |
| 1.189 | −0.28 | 0.326 | 1.792 |
| −0.159 | 0.441 | −0.439 | 1.742 |
| 0.038 | 0.077 | −0.468 | −0.564 |
| 1.807 | 4.67 | 3.508 | 4.879 |
| −0.866 | 0.636 | 3.903 | 0.036 |
| 0.01 | 2.566 | 0.723 | 0.8075 |
| 0.499 | −1.0935 | −1.49 | −2.8115 |
| 0.2155 | −0.1675 | −0.492 | 0.72 |
| 0.289 | −0.351 | −0.944 | −0.473 |
| −4.079 | −1.3735 | −0.865 | −3.599 |
| −0.9985 | −0.3475 | 0.161 | 1.5845 |
| −0.3155 | −0.5095 | 0.512 | −3.3345 |
| −0.3595 | 0.0905 | −0.483 | −1.775 |
| 0.3935 | 0.981 | 0.794 | −1.4265 |
| 2.101 | 0.4805 | −0.134 | 0.255 |
| −0.105 | 0.1585 | 1.38 | −1.8605 |
| −0.776 | 1.3635 | 1.315 | 1.3905 |
| 2.509 | −0.081 | 0.706 | 2.9455 |
| −0.8145 | 2.708 | 2.942 | 0.8475 |
| −0.6045 | 0.227 | −1.086 | −0.0345 |
| 1.31 | −1.188 | −0.765 | −0.5155 |
| −0.442 | −0.046 | −0.918 | −0.685 |
| −1.425 | −0.931 | −1.255 | −0.568 |
| −1.214 | −0.5545 | −0.227 | 2.1375 |
| −0.0665 | −1.446 | −0.839 | 1.8125 |
| −0.9395 | 1.575 | 0.86 | −0.4155 |
| 0.173 | 0.48 | 0.551 | −0.511 |
| −0.6095 | −0.2205 | 0.738 | 0.1105 |
| −0.8395 | −0.519 | 0.657 | 0.5235 |
| −0.9445 | 1.33 | 1.338 | 0.9555 |
| −0.4765 | −0.005 | −1.07 | 1.1405 |
| −0.904 | −0.401 | −0.551 | −0.7335 |
| −0.165 | 0.8635 | −0.163 | −0.578 |
| −0.5345 | −0.718 | −0.07 | −1.335 |
| 0.034 | −0.7115 | 0.093 | 0.485 |
| −0.027 | 0.134 | −0.331 | 0.372 |
| 3.259 | −0.9415 | −1.455 | −2.164 |
| 1.258 | −2.195 | −2.417 | −1.009 |
| −0.1613 | 2.0085 | 1.246 | 1.1705 |
| −1.159 | −0.9515 | −0.301 | 1.4325 |
| −0.866 | 1.3115 | −0.331 | −0.839 |
| −0.263 | 1.7695 | 0.567 | 0.256 |
| 0.191 | −2.1295 | −0.722 | −0.011 |
| −1.213 | −0.5225 | 0.012 | −1.514 |
| −0.4235 | 1.7925 | 1.283 | 1.032 |
| −0.054 | 0.043 | −0.192 | 0.8895 |
| 0.706 | 0.47 | 0.43 | −0.192 |
| −0.544 | −0.233 | 0.289 | 0.4725 |
| 0.186 | −0.004 | 0.436 | 0.061 |
| −0.068 | 0.204 | 0.527 | 0.109 |
| −0.758 | −0.2845 | 0.248 | 0.055 |
| 0.446 | 2.029 | 1.176 | 0.817 |
| −0.694 | −0.309 | −0.638 | −0.38 |
| −0.435 | 0.8935 | 1.6 | 0.838 |
| 1.311 | 1.5925 | 1.822 | 0.4675 |
| −0.421 | −1.2225 | −0.559 | 1.008 |
| −0.535 | −0.9395 | −0.56 | 1.98 |
| 2.581 | 2.1205 | 1.798 | 1.685 |
| −0.8655 | 1.128 | 2.783 | −0.3285 |
| −0.955 | −0.6315 | 0.614 | 0.1235 |
| −0.7945 | −0.2655 | −0.871 | −2.031 |
| −0.794 | 1.8775 | 1.656 | 0.571 |
| −0.3965 | −0.9315 | 0.023 | −0.012 |
| 0.183 | −0.06 | −0.438 | −0.0455 |
| −0.066 | 0.6225 | 0.593 | −0.4735 |
| 1.513 | −0.4105 | −0.034 | −0.1295 |
| −0.2438 | −1.701 | 0.858 | −0.458 |
| 1.37 | −0.422 | −1.516 | −0.4835 |
| −0.1615 | 2.502 | 0.109 | −0.232 |
| 0.303 | 2.1445 | 1.366 | 0.6245 |
| 0.609 | −0.0995 | −0.339 | 1.3435 |
| 0.6735 | 0.6455 | −0.873 | 0.453 |
| −2.24 | 0.195 | 0.801 | 2.9715 |
| 0.0615 | −0.7485 | −1.563 | 0.705 |
| −1.765 | 1.4085 | 1.141 | 2.2145 |
| −0.177 | −0.041 | −0.555 | −0.9295 |
| 0.143 | 1.1795 | 1.788 | 2.1585 |
| −0.799 | −1.391 | −1.722 | −3.3815 |
| 0.592 | 1.2755 | 0.963 | 0.784 |
| −0.5895 | −0.2735 | −0.375 | 0.4225 |
| 0.01 | 0.318 | 0.455 | 0.0915 |
| 0.144 | −0.1415 | −0.484 | 0.742 |
| 0.414 | 1.103 | 0.037 | 0.1505 |
| −0.202 | −0.012 | 0.105 | −0.4465 |
| −0.287 | 0.6415 | 1.353 | 0.6105 |
| 1.701 | 1.355 | 0.398 | 1.0185 |
| −0.356 | −1.3955 | 1.841 | −0.755 |
| −2.394 | −0.5405 | 4.641 | −1.615 |
| −0.23 | 0.3275 | −0.49 | −0.5055 |
| −0.04 | −0.5665 | −0.084 | −0.5955 |
| −0.3825 | −0.032 | −0.773 | 0.393 |
| 0.676 | 0.638 | −0.337 | −0.22 |
| −0.502 | −0.4075 | −0.153 | 0.0885 |
| −0.3765 | −0.295 | 0.828 | 2.907 |
| 0.385 | 1.0005 | −1.014 | −0.817 |
| 0.4305 | 0.499 | 0.665 | 0.9935 |
| 0.261 | 0.6305 | 0.895 | 0.2265 |
| −0.711 | −0.4795 | 0.982 | 1.164 |
| −0.985 | −0.3795 | −0.245 | −1.2965 |
| −1.764 | −0.286 | 0.821 | −0.3405 |
| −0.4595 | 1.5995 | −0.222 | −1.119 |
| −0.4247 | 1.49 | −1.284 | −0.3 |
| −0.802 | 0.2585 | 0.331 | 1.1605 |
| 0.026 | 0.3105 | 0.097 | −0.506 |
| −0.216 | 1.1645 | 0.096 | 0.46 |
| −0.304 | −1.255 | 0.578 | −0.4075 |
| 0.168 | 0.425 | −0.545 | −1.055 |
| 0.1005 | 0.3266 | −0.148 | 1.9475 |
| −1.154 | 1.3405 | 0.493 | −0.3065 |
| −0.731 | −0.886 | −0.984 | −0.26 |
| −0.8455 | −0.075 | 0.78 | 0.2715 |
| 1.08 | 0.3945 | −0.025 | −0.4075 |
| −0.2115 | −0.4985 | −0.774 | 0.038 |
| −0.195 | −0.642 | −0.723 | −1.197 |
| 0.4685 | 0.1955 | 0.313 | 0.052 |
| 0.117 | 0.8895 | −0.063 | 1.51 |
| 0.433 | −0.1065 | −0.068 | −0.5465 |
| 0.33 | 0.646 | −2.192 | 2.1455 |
| −0.3995 | 1.0335 | 0.181 | 0.402 |
| −1.631 | −0.6755 | −0.144 | −1.3265 |
| −0.316 | 0.117 | 0.235 | 0.4495 |
| −1.471 | 1.0385 | 1.975 | −1.554 |
| −0.004 | 0.481 | 0.237 | 0.822 |
| 1.311 | 0.2685 | −0.007 | 1.2705 |
| 1.258 | −2.195 | −2.417 | −1.009 |
| 0.572 | 0.4295 | 0.111 | 0.99 |
| 4.52 | 0.719 | −2.251 | 0.341 |
| −0.0195 | −0.1895 | 0.256 | 0.618 |
| −1.438 | −0.548 | −0.556 | −1.1075 |
| −0.083 | 1.46 | 1.172 | 0.0315 |
| 0.8385 | −0.4535 | 0.149 | 1.288 |
| 0.7145 | −1.4375 | −1.471 | −2.1625 |
| 1.757 | 0.633 | 3.076 | 2.4715 |
| −0.896 | −0.5895 | 0.277 | 0.3185 |
| 1.908 | 1.192 | 2.673 | 2.706 |
| 3.156 | 1.417 | 3.53 | 3.3665 |
| 2.929 | 1.1595 | 2.965 | 2.9035 |
| 0.4753 | 0.89 | 0.329 | −0.259 |
| 1.185 | −0.0365 | −0.68 | −0.097 |
| −0.362 | 0.8215 | 0.453 | 0.323 |
| 0.45 | 3.96 | 1.843 | 1.557 |
| 0.062 | −1.1775 | −1.091 | −2.0165 |
| −0.084 | 0.226 | 0.063 | −0.0525 |
| 0.188 | 0.3715 | 0.057 | 1.6385 |
| 0.277 | −0.8805 | −0.761 | −1.2325 |
| −0.0815 | −0.2805 | −0.482 | 0.8895 |
| 2.058 | −1.4345 | −2.365 | −2.112 |
| −2.19 | 0.989 | −2.377 | −0.066 |
| 1.122 | 0.2565 | −0.437 | −0.0085 |
| −0.867 | 1.7455 | −0.069 | −1.3365 |
| −1.117 | 2.369 | −0.233 | −1.4885 |
| 0.1412 | 0.152 | −0.032 | −1.3555 |
| −0.9215 | 0.4795 | −0.543 | −0.13 |
| −2.299 | 3.0925 | −0.52 | −3.312 |
| 1.815 | 4.988 | 3.458 | 2.1755 |
| 0.813 | −1.33 | −1.42 | −0.9095 |
| −0.243 | −0.2515 | −0.456 | −0.2435 |
| −0.573 | 0.4675 | 0.94 | −0.513 |
| −0.456 | −0.6715 | −0.282 | −0.329 |
| 0.001 | −1.203 | −0.573 | −0.301 |
| 0.7415 | 1.975 | 0.624 | 1.911 |
| 0.5355 | −0.5515 | −0.347 | −0.298 |
| −0.6425 | −0.9335 | −0.939 | −0.0075 |
| 0.1605 | −0.371 | −0.034 | −0.564 |
| 0.5525 | 0.495 | −0.226 | −0.4055 |
| −0.304 | −0.6245 | −0.689 | −0.6155 |
| 2.553 | 3.935 | 2.906 | 3.903 |
| −0.141 | −0.137 | 0.14 | −0.3305 |
| −1.406 | −0.4135 | −0.638 | −1.1045 |
| 0.528 | −0.2755 | 0.42 | −0.2945 |
| −0.875 | −0.226 | 0.222 | −0.57 |
| −0.201 | 1.2845 | 1.002 | 1.9355 |
| 0.231 | −0.968 | −0.394 | −1.0595 |
| −0.296 | −0.2095 | −0.384 | −0.11 |
| −2.036 | −1.1305 | −0.699 | −0.8305 |
| −1.19 | −0.3075 | 0.074 | 0.698 |
| 0.042 | −0.9015 | −0.107 | 0.082 |
| 1.609 | 1.324 | 0.729 | 0.4885 |
| −0.37 | −0.1905 | −0.834 | 0.0995 |
| −0.951 | −1.1525 | −0.536 | −1.3275 |
| −0.748 | 1.5435 | −0.227 | 0.7755 |
| 0.1685 | 0.553 | −0.342 | 2.87 |
| −0.576 | 2.429 | −0.058 | 0.533 |
| −0.178 | 0.3565 | 0.238 | 1.584 |
| 0.272 | 0.006 | 0.534 | −0.316 |
| 0.259 | 0.4035 | 0.48 | 0.491 |
| 0.045 | 1.9255 | −1.463 | −1.3115 |
| −1.639 | −0.703 | −0.133 | −1.414 |
| −0.9955 | 0.6385 | −1.125 | −2.269 |
| −0.1835 | −0.388 | −2.878 | 0.951 |
| 0.315 | 1.9 | 0.551 | 0.899 |
| 0.667 | −1.0995 | 0.294 | −1.0315 |
| −0.192 | 0.5165 | 0.208 | 0.5725 |
| −0.562 | −0.3095 | −1.056 | −0.281 |
| −0.6235 | −0.0825 | 0.02 | 1.1945 |
| −0.373 | 2.299 | −0.294 | 0.411 |
| −0.0925 | −0.519 | 0.651 | −1.1775 |
| −0.9455 | −0.094 | −0.129 | 1.065 |
| 0.3825 | 0.7435 | −0.567 | 0.2465 |
| −0.987 | 1.2565 | 1.39 | 1.549 |
| 0.33 | −0.4085 | −0.25 | 0.702 |
| 0.16 | −0.847 | 0.433 | −0.0155 |
| −0.137 | −0.19 | 0.236 | 0.243 |
| −0.0905 | 0.4405 | −0.128 | 0.635 |
| 0.6055 | −0.0755 | 0.201 | −0.388 |
| 0.7085 | −0.3575 | −0.542 | −0.4565 |
| 0.251 | −0.78 | −0.614 | 0.513 |
| −0.1055 | 1.585 | 0.012 | 0.3955 |
| −2.388 | −1.259 | 0.014 | −1.847 |
| −0.389 | 1.142 | 0.39 | −1.4875 |
| 1.483 | 0.5235 | 0.645 | 4.0945 |
| 1.258 | −2.195 | −2.417 | −1.009 |
| 0.7595 | 1.628 | 0.078 | 1.165 |
| −0.3715 | 1.1205 | 0.609 | −1.473 |
| 2.021 | 2.125 | 2.599 | 1.796 |
| 0.511 | −0.198 | −0.658 | −0.511 |
| 0.2485 | 0.112 | −0.094 | −0.0365 |
| 0.477 | 0.5005 | 0.521 | 0.1185 |
| 0.003 | −0.0515 | 0.17 | −0.2125 |
| 0.3175 | −1.2195 | −1.236 | −1.1325 |
| 0.132 | −0.882 | −1.262 | −1.0965 |
| −0.3655 | −0.486 | −1.257 | 1.417 |
| 0.543 | 1.116 | 2.047 | 0.5715 |
| 0.885 | 2.688 | 0.632 | 0.6975 |
| 0.1605 | −0.1645 | −0.388 | −1.3355 |
| 0.424 | −0.1305 | −0.443 | −0.5205 |
| −1.211 | 1.7855 | 0.031 | −0.8095 |
| 1.451 | −0.792 | −1.563 | −1.64 |
| 0.0395 | −0.9655 | −0.783 | 0.04 |
| 0.3865 | 0.596 | 1.93 | 1.2465 |
| −1.34 | 2.081 | 3.109 | 4.1745 |
| 0.318 | 0.1065 | −0.27 | −2.0265 |
| −0.5545 | 2.964 | −1.433 | 1.0265 |
| 0.451 | 1.2685 | 0.859 | 0.5 |
| −0.534 | −1.166 | −0.432 | −0.717 |
| 3.006 | −2.545 | −2.766 | 3.368 |
| 0.298 | 2.1395 | 0.347 | 0.312 |
| 2.305 | −0.221 | −0.246 | −0.85 |
| −0.8415 | −0.115 | 0.248 | 0.621 |
| −0.447 | 0.858 | 0.056 | 0.285 |
| −1.114 | 0.396 | −1.009 | 0.442 |
| 0.199 | 1.272 | 0.29 | 0.1725 |
| 0.4685 | −2.65 | −3.658 | −0.3105 |
| −0.363 | −0.787 | −0.321 | 1.1915 |
| −0.8816 | 1.223 | −0.243 | 2.599 |
| 0.842 | 0.1285 | −0.53 | −0.598 |
| −0.1745 | 0.1075 | 0.075 | −0.3015 |
| 1.722 | −0.412 | 1.199 | −1.084 |
| −0.137 | 0.151 | 0.383 | 1.07 |
| −0.636 | 2.4305 | 0.714 | 0.8535 |
| −0.365 | −0.1915 | −0.984 | −0.104 |
| 1.81 | −0.891 | −1.038 | −1.2915 |
| 0.756 | −0.231 | −0.456 | 0.595 |
| −0.188 | 0.4455 | 0.004 | −0.7715 |
| 1.974 | −1.0885 | −0.287 | −1.2485 |
| −1.035 | −0.821 | 1.425 | −0.021 |
| 0.7575 | −1.0265 | −0.923 | −0.8985 |
| 0.0695 | 1.4915 | 1.889 | 2.1135 |
| 1.522 | −1.0445 | 0.478 | −1.8205 |
| 2.57 | −1.677 | −1.285 | −1.401 |
| 2.832 | −1.102 | −0.795 | −1.2125 |
| 0.861 | −0.38 | −0.136 | 1.086 |
| 1.811 | 0.9335 | −0.473 | −0.795 |
| 2.451 | −0.283 | −1.294 | −0.4105 |
| 0.9285 | −0.9035 | −0.088 | −1.169 |
| 2.893 | −0.561 | −0.277 | −1.3155 |
| 0.297 | 0.3685 | −0.317 | −0.1895 |
| −0.82 | −0.1305 | 0.028 | 0.083 |
| −0.084 | 0.191 | 0.108 | −0.0055 |
| 0.6265 | 0.6055 | 1.123 | 2.158 |
| 1.749 | −0.938 | −0.474 | −1.3355 |
| 0.351 | 0.995 | 0.657 | 2.0075 |
| −0.3595 | 0.3165 | 0.645 | 0.724 |
| −0.5885 | 0.048 | −0.128 | 0.3665 |
| 1.053 | 0.516 | 0.041 | 0.6575 |
| −0.043 | −0.618 | −0.492 | −1.132 |
| 0.157 | 0.271 | 0.392 | −0.4565 |
| 0.328 | −1.921 | −0.553 | 0.0895 |
| −0.143 | −1.507 | −1.975 | 0.9105 |
| 0.643 | 1.639 | 3.087 | 3.738 |
| 0.476 | −1.005 | 0.074 | 1.165 |
| 1.973 | 0.0225 | −0.436 | 1.8685 |
| 0.221 | 0.236 | −0.53 | −0.0505 |
| −1.754 | 1.809 | 0.273 | 0.478 |
| 5.633 | 0.985 | −0.014 | 6.7865 |
| 0.5155 | 2.866 | 0.224 | −0.064 |
| 0.087 | 1.3495 | 0.283 | −0.1625 |
| −0.567 | 0.3145 | −0.441 | −0.0895 |
| 1.029 | 1.419 | 0.796 | 0.7795 |
| −0.244 | −0.2105 | −0.878 | 0.249 |
| 1.673 | −1.84 | −1.494 | −1.762 |
| 1.945 | 0.391 | −0.158 | 2.7375 |
| 1.087 | −0.291 | −0.662 | 0.7485 |
| −0.4725 | −0.692 | −0.003 | 0.0985 |
| 0.186 | 0.1755 | 0.302 | 0.058 |
| 1.923 | −0.7565 | −0.412 | −0.782 |
| −0.086 | 0.22 | −0.254 | −0.967 |
| 0.4345 | −0.515 | −0.773 | 1.934 |
| 1.258 | −2.195 | −2.417 | −1.009 |
| 0.995 | −0.972 | −1.166 | −0.2055 |
| 1.012 | −1.806 | −1.99 | −0.748 |
| 0.699 | 0.9385 | 1.646 | 1.6265 |
| 0.0355 | 0.633 | 0.097 | 0.6395 |
| 0.2833 | 0.3555 | 0.196 | 0.8335 |
| 2.76 | 0.4685 | −2.39 | 0.0045 |
| 0.8005 | −1.4525 | −1.406 | 1.38 |
| −0.155 | 0.271 | 0.584 | −1.663 |
| 1.246 | −0.738 | −0.104 | −0.811 |
| 0.761 | 2.454 | 1.017 | −1.0235 |
| −0.316 | −0.123 | −1.179 | 0.0055 |
| −0.57 | 1.6445 | 0.247 | 0.087 |
| −0.026 | −0.428 | 1.601 | 3.186 |
| −0.479 | 0.1405 | 0.65 | 0.7615 |
| −0.7365 | −0.0385 | 0.44 | 0.162 |
| −0.6402 | 0.473 | −2.059 | −0.552 |
| −0.747 | −0.8975 | −0.531 | −0.9905 |
| −0.366 | 1.268 | 1.22 | 1.899 |
| −1.09 | −1.0755 | −0.983 | −0.3155 |
| −1.901 | −2.4025 | −0.607 | −0.1685 |
| −0.4245 | 0.735 | 0.457 | 0.5815 |
| 0.0345 | 0.1885 | 0.084 | −0.277 |
| −0.2845 | 0.6705 | 0.338 | −0.774 |
| 0.672 | −0.075 | 0.157 | 0.8995 |
| −0.2665 | 0.502 | −0.57 | −0.0475 |
| 1.575 | 0.339 | 1.052 | 0.7435 |
| 0.5452 | 3.0325 | 0.665 | −0.209 |
| −0.704 | 0.554 | 0.334 | 0.394 |
| −0.7995 | 0.9455 | 0.351 | −0.7255 |
| 0.592 | 0.3805 | −0.054 | 0.2285 |
| −1.078 | 2.406 | 1.319 | −1.9005 |
| 0.6255 | 0.0885 | −0.046 | −0.21 |
| −1.259 | 0.0475 | 1.05 | 0.1575 |
| −0.59 | 1.518 | 1.127 | 1.5775 |
| 0.239 | 0.236 | 0.257 | −0.6655 |
| −0.073 | −0.3345 | −0.303 | −0.52 |
| 0.494 | 1.402 | −0.334 | −1.412 |
| −0.1 | −0.2335 | −0.626 | −0.202 |
| −0.0555 | −2.3995 | −1.165 | −1.875 |
| 1.343 | 0.1825 | −0.093 | −0.465 |
| 0.503 | −0.5685 | −1.992 | −0.6655 |
| −0.7745 | −1.8285 | −4.055 | −1.7385 |
| −0.2228 | 0.2635 | 0.346 | 0.7735 |
| −3.437 | 0.582 | 2.312 | −0.911 |
| −0.2625 | 0.3335 | 0.287 | 0.749 |
| 0.806 | 0.4435 | 0.792 | 0.722 |
| −0.088 | 0.735 | 0.005 | −0.682 |
| −0.2225 | 1.761 | −0.672 | 0.038 |
| 0.442 | −0.279 | −0.301 | −0.716 |
| −0.205 | −0.267 | −0.528 | −0.816 |
| −1.569 | −0.085 | −0.173 | −1.8535 |
| 0.124 | 1.1245 | −0.121 | −0.1365 |
| −0.314 | 0.3865 | 0.435 | 3.1165 |
| 0.3845 | −0.2135 | −0.08 | 0.8155 |
| 3.202 | −0.374 | −0.504 | −1.2585 |
| 0.79 | −0.0415 | −0.256 | 0.114 |
| −0.09 | 0.1555 | −0.656 | 0.0635 |
| 2.883 | −0.612 | −2.409 | −0.789 |
| −1.308 | −0.9015 | −0.569 | −0.6075 |
| −1.091 | −2.362 | −0.516 | −1.345 |
| −3.616 | −1.3425 | −1.521 | −0.8555 |
| −3.005 | −1.207 | −0.636 | 1.113 |
| 1.524 | 3.2655 | 4.402 | 4.037 |
| −0.0295 | 0.335 | 0.581 | −0.631 |
| 0.0185 | −0.366 | −1.717 | −2.117 |
| −0.25 | 1.254 | 0.219 | −0.6985 |
| 1.212 | 0.3175 | −0.289 | 0.266 |
| −0.0875 | 0.699 | −0.106 | 0.2735 |
| 0.212 | −0.059 | 0.186 | 0.533 |
| −0.339 | −0.98 | −0.269 | −1.2715 |
| 1.53 | −0.0085 | −0.491 | −0.7845 |
| −0.8165 | 1.8935 | 0.652 | 0.8695 |
| 0.872 | 0.717 | −0.035 | −0.4355 |
| −0.1955 | 0.024 | −0.181 | −0.1725 |
| 1.539 | 1.002 | 1.155 | 1.201 |
| 0.0485 | 1.025 | 0.073 | 0.459 |
| −1.102 | 1.4085 | 2.009 | 0.716 |
| 0.064 | 0.2845 | −0.039 | 0.5955 |
| 0.2565 | −0.3575 | −0.529 | −0.0865 |
| −0.327 | 0.164 | 0.191 | −0.502 |
| −0.988 | −0.8975 | −1.212 | −0.9825 |
| −0.158 | 0.182 | −0.45 | 0.1875 |
| 0.456 | −0.192 | 1.703 | 2.505 |
| 0.9445 | −2.8085 | −3.064 | −0.213 |
| −0.3875 | −0.02 | −0.312 | −0.164 |
| 0.3985 | −1.828 | −0.548 | −0.751 |
| 1.258 | −2.195 | −2.417 | −1.009 |
| −0.6015 | −0.517 | −4.208 | −3.3 |
| −0.5555 | −0.9515 | −0.47 | −0.536 |
| −0.059 | −0.2335 | 0.094 | −1.5255 |
| −0.255 | −0.163 | 0.082 | 0.013 |
| −2.212 | 0.934 | −0.228 | −0.457 |
| −0.0498 | −0.403 | 0.399 | −0.51 |
| 0.278 | −0.3295 | −0.151 | 0.0455 |
| −1.548 | −1.0785 | −0.132 | −0.718 |
| −0.843 | 0.975 | 0.285 | 0.1805 |
| −1.333 | 0.1645 | −0.262 | −1.92 |
| 0.157 | 0.787 | 0.912 | 0.7205 |
| 0.539 | 0.842 | 0.532 | −0.5215 |
| −0.1215 | −0.0455 | −1.146 | −0.616 |
| −1.383 | 1.6555 | −0.623 | −1.157 |
| 1.286 | 1.0675 | −0.628 | −1.1715 |
| −1.695 | −0.7475 | −0.694 | −1.412 |
| 0.7325 | −0.573 | −0.546 | −1.187 |
| −1.295 | −0.134 | 0.158 | 0.125 |
| −0.3065 | −0.3555 | −0.93 | −0.444 |
| −0.036 | −0.6375 | −0.22 | −0.9395 |
| −0.659 | 0.6055 | −0.874 | 0.161 |
| −0.55 | −0.0435 | −1.151 | −1.4135 |
| −0.615 | 1.272 | 0.699 | 0.943 |
| 0.139 | 0.113 | 0.517 | 0.871 |
| 0.9375 | 0.016 | −0.482 | 0.052 |
| −0.518 | 1.6575 | 0.4 | 0.592 |
| −1.604 | 0.0505 | −0.117 | 0.633 |
| −0.27 | −0.0375 | 1.021 | 2.514 |
| −0.378 | 0.8635 | 0.945 | 1.0105 |
| −0.2245 | 0.5865 | 0.586 | 0.614 |
| −0.864 | 1.546 | 1.2 | 1.0445 |
| −0.2045 | −0.757 | −0.578 | 4.301 |
| 0.112 | 0.007 | 0.056 | 0.278 |
| 0.648 | 0.528 | −0.85 | −0.505 |
| −0.302 | −1.231 | −0.767 | −1.8945 |
| −1.568 | −0.063 | 0.159 | −0.6025 |
| 0.126 | −0.6125 | −0.071 | −1.632 |
| −0.606 | −0.22 | 0.422 | 0.1305 |
| 0.0765 | −0.7335 | −3.021 | −0.9475 |
| 0.506 | −0.112 | −0.499 | −2.267 |
| −0.703 | −1.1285 | −1.085 | −1.008 |
| 0.837 | −0.3865 | −1.178 | −1.145 |
| 0.579 | −0.865 | −1.271 | −1.581 |
| −0.423 | 1.1645 | 0.843 | 1.1015 |
| −0.2007 | 0.6735 | 0.287 | 0.484 |
| −0.1645 | −0.015 | −0.92 | −1.4005 |
| 0.239 | −0.257 | −0.289 | 0.47 |
| 0.702 | 0.609 | −0.606 | −0.0455 |
| −0.474 | 0.725 | −0.361 | 0.2915 |
| 0.057 | −0.4655 | −0.824 | 0.484 |
| −0.2695 | −0.1425 | −0.004 | −0.185 |
| 1.952 | 0.32 | −0.066 | 0.5295 |
| 2.004 | 1.2525 | 0.596 | 1.7725 |
| 0.329 | 1.755 | 0.496 | 0.9255 |
| −1.46 | −1.8745 | −2.395 | −2.1025 |
| 0.12 | −0.365 | −0.104 | 0.619 |
| −3.066 | −0.699 | −1.365 | −1.655 |
| 0.266 | −1.8445 | −0.974 | −2.79 |
| 0.8095 | −0.6555 | −0.496 | 0.6745 |
| −1.228 | −1.065 | −1.347 | −1.872 |
| −2.1 | −1.1515 | −0.051 | −1.493 |
| −1.618 | −0.8285 | −1.608 | −1.358 |
| 1.029 | 3.664 | 3.107 | 2.519 |
| −0.603 | 0.1525 | −0.65 | 1.2495 |
| 1.222 | −0.961 | −0.415 | −1.1035 |
| 0.1985 | 0.025 | 0.063 | −0.936 |
| −0.016 | −0.088 | −0.037 | −0.467 |
| 0.171 | 1.243 | 0.515 | 0.8075 |
| −0.076 | −1.238 | 0.024 | −1.8455 |
| 0.8385 | −0.558 | −0.621 | 0.03 |
| 0.237 | 0.4055 | 0.225 | 0.185 |
| −0.356 | 3.1915 | 1.368 | 1.5945 |
| −0.4095 | 0.351 | −0.18 | 0.248 |
| −0.5988 | −1.891 | −0.137 | −3.7605 |
| 0.1835 | −0.2595 | −0.046 | 1.556 |
| −0.0225 | −1.1765 | −0.756 | −1.344 |
| 0.15 | 2.2665 | 0.157 | 0.5825 |
| 0.25 | 0.614 | −0.475 | −1.006 |
| −0.055 | −0.3425 | 1.185 | −0.221 |
| −0.203 | −0.296 | 0.129 | −1.6045 |
| −0.052 | 0.6085 | −0.349 | −0.9045 |
| 3.695 | −0.05 | −0.379 | −1.5885 |
| −0.305 | −0.395 | −0.413 | 0.382 |
| −0.453 | −0.6315 | 0.577 | −0.9615 |
| 0.4377 | −0.052 | −0.404 | −0.111 |
| −1.021 | 1.2955 | −0.446 | −0.8295 |
| 1.258 | −2.195 | −2.417 | −1.009 |
| 0.093 | −0.0545 | −0.045 | −0.2265 |
| 3.557 | −0.7755 | −0.965 | −0.0635 |
| 0.566 | 0.661 | 0.632 | 0.4875 |
| 0.7825 | −0.265 | −0.874 | −0.7085 |
| 0.651 | −0.309 | 0.084 | −0.2945 |
| −0.0998 | −0.668 | −0.506 | −1.357 |
| 0.6065 | −1.505 | −0.422 | −1.117 |
| −0.006 | −0.1585 | −0.134 | −0.238 |
| −0.1995 | −0.3885 | −0.524 | −1.0605 |
| 1.07 | 0.672 | −0.839 | −0.4365 |
| −0.256 | −0.031 | −0.346 | −0.277 |
| −0.039 | −0.7895 | −0.478 | −1.6515 |
| 0.668 | −0.8305 | −0.529 | −0.305 |
| 0.569 | 0.4485 | −0.113 | −0.073 |
| −2.94 | −1.9705 | −2.877 | −0.377 |
| 0.4005 | 1.131 | −0.308 | −0.8855 |
| 0.308 | −0.2965 | −0.532 | −0.599 |
| 0.513 | 0.276 | 0.048 | 0.11 |
| −0.1303 | 1.1625 | 0.394 | −0.49 |
| 0.0405 | −0.601 | 0.093 | −1.292 |
| −3.945 | −1.616 | −2.542 | −2.2225 |
| 0.1155 | 0.7615 | −0.023 | 0.355 |
| 0.185 | 0.6515 | −0.016 | 1.0175 |
| −0.2135 | 1.007 | 0.643 | −0.217 |
| −1.624 | −0.9095 | −0.505 | −0.382 |
| −0.167 | 1.4065 | 0.878 | 0.413 |
| 0.1305 | −0.424 | 3.412 | 1.454 |
| 0.318 | −0.3175 | 1.727 | 0.354 |
| 0.0027 | 3.5815 | 0.998 | 2.855 |
| −1.174 | −0.6585 | −0.625 | −1.088 |
| 0.495 | −1.6285 | 0.224 | 0.1115 |
| 0.073 | 0.0875 | 0.08 | −0.338 |
| 0.624 | 0.372 | −0.334 | −0.7245 |
| 1.016 | 2.995 | 4.379 | 4.205 |
| 2.825 | 0.5805 | 0.483 | 0.44 |
| −1.012 | −0.7655 | −1.183 | −0.726 |
| 0.0008 | 2.223 | 1.215 | 0.7945 |
| −1.095 | −0.6735 | 1.378 | 1.4885 |
| −1.314 | 1.2115 | 1.778 | 1.6215 |
| 0.187 | 1.1785 | 0.34 | 0.023 |
| 0.129 | 0.8875 | −0.082 | −0.076 |
| −0.438 | −0.422 | 4.378 | −0.255 |
| −1.269 | 0.5665 | −0.315 | −0.3865 |
| 0.406 | −0.0935 | −0.401 | 0.323 |
| −0.447 | −0.1885 | −2.363 | 0.603 |
| −0.1475 | 0.087 | 1.146 | 1.613 |
| 0.008 | 1.157 | 0.416 | 0.18 |
| 0.1683 | 0.204 | 0.161 | −0.1645 |
| −2.551 | −0.7175 | −2.65 | −0.331 |
| −0.0715 | −0.052 | 0.782 | −1.0715 |
| −0.822 | 2.4295 | −1.8 | −0.271 |
| 0.3185 | −0.7575 | 0.742 | 0.6175 |
| 0.106 | −0.4285 | −0.535 | −1.0785 |
| 0.396 | 0.302 | 3.594 | −2.4045 |
| 2.897 | 1.321 | −0.094 | −0.5665 |
| −0.4005 | −0.0165 | −0.148 | −0.341 |
| 0.088 | −0.0185 | 0.502 | 0.9815 |
| −0.0725 | 0.598 | 0.435 | 0.5235 |
| −0.453 | 2.1895 | 1.028 | 1.3975 |
| 0.1205 | −0.6335 | −0.556 | −0.0265 |
| 1.109 | −0.3175 | −0.404 | −0.2365 |
| 0.075 | 0.219 | 0.107 | 0.956 |
| 1.099 | −0.454 | −0.979 | −1.6235 |
| −0.5965 | −0.0785 | −0.659 | 1.0165 |
| −0.47 | −0.61 | −0.05 | 1.301 |
| −0.001 | 0.5665 | 0.425 | 0.12 |
| 0.0175 | −0.4025 | −1.786 | 1.649 |
| 0.2975 | 1.2045 | 0.392 | 0.259 |
| −0.073 | −0.346 | −0.227 | −0.143 |
| 0.7055 | −0.069 | −1.145 | 0.0225 |
| 0.649 | −0.702 | −0.587 | −2.553 |
| 0.7393 | −2.376 | −1.985 | −1.3105 |
| −0.26 | −0.6305 | −0.846 | −0.398 |
| −0.071 | 0.959 | 0.153 | −0.153 |
| −0.772 | −1.0275 | 0.43 | 0.543 |
| 0.5245 | −2.531 | −2.535 | −1.3725 |
| 5.09 | 2.1575 | 2.119 | 2.042 |
| −0.1265 | 0.0615 | 0.122 | 0.242 |
| −1.112 | 0.026 | 0.051 | −0.471 |
| 0.059 | −0.5865 | −0.511 | 0.236 |
| −0.6 | −1.127 | −0.983 | 1.267 |
| −0.37 | −1.0225 | −0.685 | −1.311 |
| −0.0995 | −1.002 | −0.244 | −0.066 |
| 0.599 | 0.684 | 0.42 | −0.6205 |
| 0.187 | 0.409 | −0.203 | −1.326 |
| 0.014 | −0.8295 | −0.726 | −0.5525 |
| 1.258 | −2.195 | −2.417 | −1.009 |
| −0.0503 | −0.1615 | −0.135 | 0.1335 |
| −1.109 | −0.5465 | −0.516 | −1.0545 |
| −1.272 | −0.921 | −0.346 | −1.3355 |
| 0.0655 | −0.1975 | −0.986 | −0.2275 |
| 0.001 | 0.0235 | −0.019 | −0.8465 |
| 0.4015 | 0.238 | −0.193 | −1.988 |
| 0.6013 | 1.755 | 0.138 | −0.9295 |
| 0.3315 | −0.5515 | −0.12 | −0.875 |
| −1.403 | 0.7235 | 2.769 | −0.2935 |
| 0.162 | 1.923 | 0.454 | 1.708 |
| −0.7682 | 0.127 | 0.029 | −1.4335 |
| −0.9525 | −0.757 | −1.482 | −1.66 |
| −0.444 | −1.1955 | −1.525 | −1.488 |
| −0.144 | 0.239 | 0.199 | 2.2265 |
| 0.887 | 1.0615 | 1.824 | 1.4095 |
| −0.2395 | 0.2955 | 0.942 | 0.5475 |
| −1.49 | −1.2625 | 0.85 | −1.26 |
| −0.4485 | 0.276 | 0.407 | 0.81 |
| 1.312 | 0.758 | 1.234 | −0.381 |
| −0.698 | 0.942 | −0.729 | −0.1515 |
| −0.888 | −0.461 | −1.076 | −1.0225 |
| −0.489 | −0.5475 | −1.196 | −0.409 |
| −1.768 | 0.3105 | −0.292 | 2.542 |
| 0.55 | −0.0685 | −0.618 | −0.368 |
| −0.6155 | −0.3565 | 0.412 | 0.3365 |
| 1.897 | 2.9345 | 1.31 | 2.011 |
| −1.302 | −2.2145 | −2.166 | −1.5405 |
| 1.083 | −0.7245 | −0.877 | −0.1575 |
| −0.693 | −2.0745 | −0.995 | −1.2275 |
| −1.139 | 0.1695 | 0.043 | −2.5715 |
| −0.871 | −0.4765 | −0.328 | 1.1625 |
| 0.1615 | 0.0875 | −0.424 | 0.263 |
| 0.023 | −0.7575 | −0.734 | −1.245 |
| −0.49 | −0.227 | −0.013 | 0.4805 |
| 2.479 | 0.062 | −3.592 | −3.1325 |
| 0.022 | 0.071 | −0.526 | −0.3445 |
| 0.017 | −0.5225 | −0.412 | −2.175 |
| 0.74 | −0.366 | −1.256 | −0.6035 |
| −0.439 | 0.3285 | 2.682 | 3.375 |
| −1.1 | −0.0635 | 0.501 | −1.2335 |
| 0.708 | −1.443 | −1.502 | 0.689 |
| 0.125 | −0.5515 | 0.76 | −0.29 |
| −0.487 | −0.1195 | −0.027 | 0.0075 |
| 0.195 | 0.6225 | 1.067 | 0.6315 |
| −0.112 | −0.2525 | −1.651 | −1.4895 |
| −0.572 | 0.41 | 0.161 | −0.694 |
| 0.1405 | −1.2325 | 0.537 | −0.8745 |
| −0.1772 | −0.244 | 1.054 | 0.398 |
| −0.139 | −0.8765 | −0.591 | 0.4255 |
| 0.2655 | −1.086 | −0.89 | −0.5695 |
| −1.624 | 0.6645 | 0.679 | 1.418 |
| 1.667 | −0.621 | −1.587 | 2.1335 |
| −0.7145 | 0.1565 | 0.127 | 0.7915 |
| 2.065 | −0.1645 | −0.787 | 0.525 |
| −0.185 | −1.84 | −1.408 | −2.8535 |
| 0.524 | −0.073 | 0.332 | −0.1375 |
| −1.759 | −0.5705 | 1.634 | 2.407 |
| −0.79 | 0.406 | 0.188 | 0.6265 |
| 0.0605 | 1.099 | −0.009 | −0.37 |
| 0.665 | 1.3855 | 0.026 | 0.6925 |
| 0.2408 | 1.985 | 1.488 | 0.9415 |
| 0.173 | −0.8835 | −0.036 | 1.1305 |
| −0.643 | 3.577 | 2.12 | 0.2175 |
| 0.993 | 1.328 | 3.69 | −0.2495 |
| −0.202 | −0.2435 | 0.329 | −0.211 |
| 0.962 | 0.1635 | 1.083 | 0.3975 |
| −0.769 | −0.2215 | 0.48 | −1.7465 |
| 0.574 | 0.003 | −0.613 | −0.5195 |
| −0.313 | 0.628 | 0.059 | −0.764 |
| −0.5545 | −0.3295 | −0.571 | −0.9955 |
| 1.258 | −0.5155 | −0.164 | −0.216 |
| 0.684 | 0.483 | −0.508 | 1.0075 |
| −0.8415 | −0.724 | −0.676 | −0.6555 |
| 0.51 | −0.6785 | −0.194 | −0.833 |
| 0.8362 | 0.1435 | −0.636 | −0.646 |
| 0.5955 | 0.762 | −0.496 | −0.862 |
| −1.793 | 0.8615 | −1.823 | −1.6915 |
| −1.182 | 0.2905 | −0.776 | 1.7525 |
| 0.4095 | 2.481 | 1.157 | 1.1395 |
| −0.6985 | 2.3805 | −0.842 | 0.276 |
| 1.963 | −0.7905 | −0.1 | 2.7755 |
| 0.917 | 0.088 | −0.833 | 0.775 |
| 2.632 | 1.3155 | −0.409 | 2.578 |
| 0.065 | −0.5745 | −1.127 | −0.1285 |
| 1.034 | −0.477 | −0.576 | −0.702 |
| 3.766 | −0.8315 | −1.652 | −1.013 |
| 1.258 | −2.195 | −2.417 | −1.009 |
| −0.125 | 0.493 | −0.171 | −2.113 |
| 0.787 | 0.698 | 1.169 | 1.242 |
| 0.8595 | 0.485 | −0.604 | −1.01 |
| 1.611 | −0.8495 | −0.316 | −0.801 |
| 1.297 | 2.1275 | 0.711 | 0.5135 |
| −0.6265 | 2.428 | −0.922 | −0.212 |
| 0.422 | −0.616 | 0.084 | −0.6035 |
| 0.3862 | 0.456 | 1.408 | 0.931 |
| −0.713 | 0.488 | 1.319 | 1.7595 |
| 0.362 | −1.458 | −1.975 | 1.9145 |
| 0.0705 | −0.617 | −1.067 | 1.604 |
| 0.7085 | −1.876 | −0.311 | −0.2115 |
| −0.016 | −0.2585 | 0.169 | 0.012 |
| −0.689 | −1.204 | 0.515 | 0.478 |
| −0.3525 | −1.749 | −1.205 | −2.084 |
| 0.174 | 0.107 | 0.516 | 0.092 |
| −1.02 | −0.381 | 0.494 | −0.2645 |
| −0.003 | −1.3185 | −1.966 | −2.576 |
| 0.899 | 0.877 | 1.257 | 0.8625 |
| −1.854 | 1.7755 | 1.337 | −0.7485 |
| 0.143 | 0.6555 | 1.667 | 3.1895 |
| 0.0525 | 0.4065 | 0.92 | 0.239 |
| −1.206 | −0.464 | −0.28 | −1.7025 |
| −0.4585 | 1.0845 | 1.514 | 3.372 |
| −0.132 | 1.217 | 0.066 | 0.6395 |
| 0.588 | −0.8915 | −0.968 | −0.9995 |
| −0.244 | 0.138 | 0.102 | −0.242 |
| 0.3267 | 2.6225 | 0.022 | 0.75 |
| 0.8218 | 2.2175 | −0.791 | −0.2545 |
| 0.141 | 0.742 | −0.547 | −0.675 |
| −0.267 | 0.4 | −0.703 | −0.2935 |
| −0.0045 | 3.729 | 0.765 | 5.2815 |
| 0.8805 | 0.0815 | 0.107 | 0.613 |
| 0.9035 | −0.317 | 0.77 | 0.705 |
| 0.335 | −0.517 | −0.479 | −0.909 |
| −0.828 | 0.266 | −0.181 | −2.171 |
| −0.3865 | 0.0135 | 0.633 | −0.404 |
| −0.1005 | 1.863 | 0.003 | −0.7615 |
| −0.215 | 0.2095 | 0.84 | −0.5155 |
| −1.069 | −0.235 | 0.737 | 0.3165 |
| −0.183 | 1.0175 | 1.979 | 0.416 |
| −3.524 | −0.466 | −1.15 | −2.8765 |
| −1.077 | −0.454 | −0.422 | −0.5775 |
| 0.5598 | 0.222 | 0.215 | −1.7235 |
| 0.342 | 0.215 | −0.416 | 0.069 |
| −0.3125 | −0.7235 | −0.137 | 0.2315 |
| −0.096 | 1.539 | −0.996 | −2.0415 |
| 0.74 | −1.082 | 0.546 | −0.9845 |
| −0.0175 | 0.2045 | −0.274 | 2.644 |
| −0.747 | −0.377 | 0.065 | 0.073 |
| −0.478 | −1.356 | −2.276 | −2.133 |
| −1.275 | 1.23 | 0.382 | 1.1065 |
| −0.267 | −0.4675 | −0.03 | −0.4625 |
| 1.218 | 0.121 | −0.235 | −0.726 |
| 0.075 | 0.642 | 0.35 | 0.135 |
| 3.388 | 0.558 | 3.188 | 4.885 |
| −1.064 | −2.437 | −2.775 | 3.109 |
| 0.5685 | −0.9105 | −0.456 | −0.8495 |
| −0.3355 | 0.4465 | 0.129 | 0.2645 |
| −0.8415 | −0.6935 | 1.477 | 0.7645 |
| 0.816 | 0.407 | 0.333 | 0.155 |
| −0.055 | 0.727 | 0.689 | 0.7025 |
| 0.1845 | 1.076 | 0.777 | 1.251 |
| 0.4375 | 0.7615 | 0.94 | 0.267 |
| −0.294 | −0.1115 | −0.937 | 0.4265 |
| 0.057 | 0.547 | 0.108 | 2.4905 |
| 0.567 | 0.1465 | −0.629 | 0.22 |
| −0.117 | 1.4275 | 0.96 | −0.4215 |
| 0.4475 | −4.686 | −4.499 | −4.292 |
| 0.2147 | 0.1585 | 0.524 | 3.423 |
| 0.102 | 0.69 | 0.344 | 0.1975 |
| 1.074 | −0.6955 | −0.588 | 2.0955 |
| −0.53 | −0.098 | −1.159 | 0.1365 |
| −1.467 | −0.263 | −0.688 | 0.8055 |
| 0.186 | 0.1015 | −0.852 | −0.089 |
| 0.958 | −0.81 | −1.177 | −0.894 |
| −0.0853 | −1.5085 | −0.579 | −1.9215 |
| −0.261 | 0.0105 | 0.231 | 0.17 |
| 1.151 | 0.7545 | 0.917 | 2.016 |
| −0.003 | 1.3845 | 0.202 | −0.0165 |
| 1.179 | 0.631 | 0.323 | 0.6045 |
| 0.884 | 0.264 | 0.551 | 0.23 |
| −1.198 | −0.8815 | −2.198 | −1.421 |
| 0.381 | 0.507 | −0.247 | 0.267 |
| 0.088 | −0.05 | 0.208 | −0.0065 |
| −0.693 | 0.7775 | 1.48 | 0.204 |
| 1.258 | −2.195 | −2.417 | −1.009 |
| −0.6295 | 1.447 | 0.401 | 0.049 |
| −0.048 | 0.1175 | 0.401 | 0.926 |
| 0.147 | −0.0745 | −0.954 | −0.186 |
| 0.602 | 0.35 | 0.003 | −0.149 |
| −0.01 | −0.0375 | −0.039 | −0.191 |
| −0.0605 | 0.2575 | 0.896 | −0.4445 |
| −1.171 | −0.3635 | −1.074 | −0.9295 |
| 0.287 | 0.919 | 0.202 | 1.497 |
| 0.607 | 0.5715 | 0.004 | 0.949 |
| 0.454 | −0.2315 | −0.104 | 1.327 |
| −1.064 | −0.6975 | −0.317 | −0.4125 |
| −0.032 | 1.912 | −0.452 | 0.495 |
| 0.1128 | 0.2275 | 0.448 | 0.9535 |
| −0.038 | −0.0855 | −0.273 | −0.8535 |
| 0.284 | 0.454 | −0.62 | −0.3355 |
| 1.108 | 0.9605 | 0.077 | 1.368 |

[symbol]

indicates data missing or illegible when filed

| P3 minimized | | P2 minimized |
|---|---|---|
| Intrinsic gene list | | Intrinsic gene list |
| ASF1A | | ACADSB |
| BLVRA | B3GNT5 | BF |
| BTG3 | COX6C | C5ORF18 (=DP1) |
| C10orf7 | ERBB2 | CDK2AP1 |
| C16orf45 | ESR1 | CX3CL1 |
| CaMKIINalpha | FOXC1 | CYB5 |
| CDH3 | FZD7 | DSC2 (ESTs) |
| CHI3L2 | GATA3 | EGFR |
| CSDA | GRB7 | FLJ14525 |
| CTPS | GSTP1 | FOXA1 |
| FABP7 | KIT | GARS |
| FBP1 | KRT17 | HSD17B4 |
| FLJ10980 | S100A11 | KIAA0310 |
| GSTM3 | SLC39A6 | KRT5 |
| HIS1 | XBP1 | NAT1 |
| ID4 | | PGR |
| IGBP1 | | PLOD1 |
| INPP4B | | PTP4A2 |
| SEMA3C | | RABEP1 |
| SLC5A6 | | RARRES3 |
| TCEAL1 | | SDC2 |
| TFF3 | | SLPI |
| TMSB10 | | SMA3 |
| TP53BP2 | | TAP1 |
| VAV3 | | TRIM29 |
| WWP1 | | |

| Proliferation genes | Proliferation genes |
|---|---|
| BUB1 | BIRC5 |
| MKI67 | BUB1 |
| MYBL2 | CENPF |
| STK6 | CKS2 |
| TOP2A | FAM54A (=DUFD1) |
| | GTPBP4 |
| | HSPA14 |
| Housekeeper genes | |
| ACTB | MKI67 |
| MRPL19 | MYBL2 |
| PSMC4 | NEK2 |
| PUM1 | PCNA |
| SF3A1 | STK6 |
| | TOP2A |
| | TTK |
| | Housekeeper genes |
| | MRPL19 |
| | PSMC4 |
| | PUM1 |

TABLE 21

| | | | | Forward Sequence |
|---|---|---|---|---|
| GENE | Other Symbol | Gene Name | Location | (SEQ ID NO: 29-240) |
| ACTB | PS1TP5BP1 | actin, beta | 7p15-p12 | TTCCTGGGCATGGAGTC |
| GAPDH | GAPDH, GAPD | glyceraldehyde-3- | 12p13 | TGGAAGGACTCATGACCACA |
| | | phosphate dehydrogenase | | |
| GUSB | | glucuronidase, beta | 7q22 | ACTATGCAGCAGACAAGG |
| RPLP0 | 36B4, P0, L10E, | ribosomal protein, large, | 12q24.2 | TCTACAACCCTGAAGTGCT |
| | RPPO, PRLP0 | P0; also known as 60S | | |
| | | acidic ribosomal protein | | |
| | | P0, [symbol] | | |
| TFRC | CD71, TFR1 | transferrin receptor | 3q26.2-qter | GACTATTGCTGTGATCGTCT |
| | | (p90, CD71 | | |
| MRPL19 | MRP-L15, RPML15, | Mitochondrial ribosomal | 2p112-q11.2 | GGAAGAGGACTTGGAGCTACT |
| | KIAA0104, RLX1 | protein L19 | | |
| PSMC4 | TBP7, S6, MIP224 | proteasome (prosome, | 19q13.11-q13.13 | ACCTGGCTGTGGGAAGA |
| | | macropain) 26S subunit, | | |
| | | ATPase, 4 | | |
| PUM1 | PUMH1, KIAA0099 | KIAA0099 pumilio | 1p35.2 | CGGGAGATTGCTGGACATATAA |
| | | bonaolog 1 (Drosophila) | | |
| SF3A1 | SF3a120, SAP114, | splicing factor 3a, | 22q12.2 | CGATGATGAGGTGTACGC |
| | PRPF21, Prp21 | subunit 1, 120 kDa | | |
| SLC7A6 | y+LAT-2, KIAA0245, | solute carrier family 7 | 16q22.1 | TGGCACTCATCTACCTCATC |
| | LAT3, LAT-2 | (cationic amino acid | | |
| | | transporter, y+system), | | |
| | | member 6 | | |
| S100A11 | S100C | S100 calcium binding | 1q21 | GCGCACAGAGCTCTCAG |
| | | protein A11 (calgizzarin) | | |
| ASF1A | DKFZP547E2110, CIA | ASF1 anti-silencing | 6q22.31 | TCCACCAGTAAAACCAGACTT |
| | | function 1 homolog A | | |
| | | (S. cerevisiae) | | |
| BLVRA | BLVR | biliverdin reductase A | 7p14-cen | GCTGGCTGAGCAGAAAG |
| BTG3 | ANA, MGC8928, TOB5, | BTG family, member 3 | 21q21.1-q212 | AGCCGCAAGTCCTGTGTA |
| | TOB55, TOFA | | | |
| C10orf7 | D123 | chromosome 10 open | 10p13 | CCCCAAGGGATGCGTAT |
| | | reading frame 7 | | |
| C16orf45 | FLI32618 | chromosome 16 open | 16p13.2 | GCTCAGGTTCATGATGGAT |
| | | reading frame 45 | | |
| CAMK2N1 | CaMKIINalpha, | calcium/calmodulin- | 1p36.12 | GAGCAAGCGGGTTGTTAT |
| | ICAP-1 alpha | dependent protein kinase | | |
| | | II inhibitor 1 | | |
| CHI3L2 | YKL-39, YKL39 | chitinase 3-like 2 | 1p13.3 | TCCCAAACTGAAAATTCTCT |
| | | | | TGTC |
| CSDA | dbpA, ZONAB, CSDA1 | cold shock domain | 12p13.1 | TCGCTCACGGGTCTTAC |
| | | protein A | | |
| FABP7 | B-FABP, BLBP | fatty acid binding | 6q22-q23 | GCAAAATGGTTATGACCCTTAC |
| | | protein 7 | | |
| HEXIM1 | CLP-1, HIS1, MAQ1, | hexamethylene bis- | 17q21.31 | TCCGAGGCCAGTAAGTTG |
| | EDG1 | acetamide inducible 1 | | |
| ID4 | | inhibitor of DNA binding | 6p22-p21 | CCCAACAAGAAAGTCAGCAA |
| | | 4, dominant negative | | |
| | | helix-loop-helix protein | | |
| IGBP1 | alpha 4 | immunoglobulin (CD79A) | Xq13.1-q13.3 | TGCCGAAATGTTATCGCAG |
| | | binding prorein 1 | | |
| INPP4B | MGC132014 | inositol polyphosphate- | 4q31.21 | GGCAGCACTTTCTTCCTACA |
| | | 4-phosphatase, type II, | | |
| | | 105 kDa | | |
| SLC5A6 | SMVT | solute carrier family 5 | 2p23 | TCTTCAGCGGCTCTCTCA |
| | | (sodium-dependent viatamin | | |
| | | transporter), member 6 | | |
| TMSB10 | | thymosin, beta 10 | 2p11.2 | TGGCAGACAAACCAGACAT |
| WWP1 | ATP5, DKFZP434D2111 | WWdomain containing E3 | 8q21 | CTTGCTCACTTCCGTTATTTG |
| | | ubiquitin protein ligase 1 | | |
| BAG1 | | BCL2-associated athanogene | 9p12 | CTGGAAGAGTTGAATAAAGAGC |
| GSTM1 | MU, H-B | glutathione S-transferase | 1p13.3 | GGACGCTCCTGATTATGAC |
| | | M1 | | |
| MMP11 | | matrix metallopeptidase 11 | 22q11_2 | AGGGGTGCCCTCTGAGAT |
| | | (stromelysin 3) | | |
| CD68 | SCARD1, macrosialin | CD68 antigen | 17p13 | GGGCAGAGCTTCAGTTG |

| C17orf37 | MGCl4832, ORB3, | chromosome 17 open reading | 17q12 | TCTCCAGCCACCTCATAC |
|---|---|---|---|---|
| | XTP4 | frame 37 | | |
| TCAP | LGMD2G, T-cap, | titin-cap (telethonin) | 17q12 | GTGGTGCCTGTCAGCAA |
| | TELE, telethonin, | | | |
| | CMD1N | | | |
| EMSY | Cl1or130 | chromosome 11 open reading | 11q13.5 | GCTCCCAGCTTCTTCAGAGA |
| | | frame 30 | | |
| IGFBP2 | IBP2 | insulin-like growth factor | 2q33-q34 | GAGTGCTGGTGTGTGAAC |
| | | binding protein 2, 36 kDa | | |
| MDM2 | HDM2 | Mdm2, transformed 3T3 cell | 12q13-q14 | GACTCCAAGCGCGAAAAC |
| | | double minute 2, p53 | | |
| | | binding protein | | |
| PTEN | MMAC1, TEP1, PTEN1 | phosphatase and tensin | 10q23 | GGGAAGTAAGGACCAGAGACAA |
| | | homolog (mutated in | | |
| | | multiple advanced | | |
| | | cancers 1) | | |
| TP53 | P53 | tumor protein p53 | 17p13.1 | AGGCCTTGGAACTCAAGGAT |
| | | (Li-Fraumeni syndrome) | | |
| CDC6 | | CDC6 cell division cycle 6 | 17q21.3 | GTAAATCACCTTCTGAGCCT |
| | | honaolog (S. cerevisiae) | | |
| KIF13B | GAKIN, KIAA0639 | kinesin family member 13B | 8p21 | GCCCTCTCTGTTTCTCCC |
| MUC1 | CD227 | mucin 1, cell surface | 1q21 | GATCGTAGCCCCTATGAGAC |
| | | associated | | |
| TK1 | | thymidine kinase 1, soluble | 17q23.2-q25.3 | CAGCTTCTGCACACATGACC |
| CLDN7 | CEPTRL2, CPETRL2 | claudin 7 | 17p13 | GGGAGACGACAAAGTGAAG |
| FGFR4 | JTK2, CD334 | fibroblast growth factor | 5q33-qter | GATCGTCCTGCAGAATCTC |
| | | receptor 4 | | |
| PDSS1 | TPT, COQ1, TPRT | prenyl (decaprenyl) | 10p12.2 | ACTCGGTTGGAGAGACT |
| | | diphosphate synthase, | | |
| | | subunit 1 | | |
| AKT3 | PKBG, RAC-gamma, | v-akt murine thymoina | q43-44 | TGGATTTACLATCCCCTCAA |
| | PRKBG | viral oncogene hornolog 3 | | |
| | | (protein kinase B, gamma) | | |
| AVEN | PDCD12 | apoptosis, caspase | 15q13.1 | GGACCTGAAATCCAAGGAAGAT |
| | | activation inhibitor | | |
| BCL2A1 | GRS, BFL1, BCL2L5 | BCL2-related protein A1 | 15q24.3 | AACGTCCAGAGTGCTACA |
| CA9 | MN | carbonic anhydrase IX | 9p12 | TCAGCCGCTACTTCCAATA |
| CDKNIB | KIP1, P27KIP1 | cyclin-dependent kinase | 12p13.1-p12 | CCCTAGAGGGCAAGTACGAGT |
| | | inhibitor 1B (p27, Kip1) | | |
| CFLAR | CASH, Casper, | CASP8 and FADD-like | 2q33-q34 | CTCACCGTCCCTGTACCTG |
| | CLARP, FLAME, FLIP, | apoptosis regulator | | |
| | I-FLICE, [symbol] | | | |
| FIGF | VEGF-D, VEGFD | c-fos induced growth | Xp2231 | ACTCTCATCTCCAGGAACC |
| | | factor (vascular | | |
| | | endothelial growth | | |
| | | factor D) | | |
| IGF1 | JTKI3 | insulin-like growth | 15q25-q26 | GCAGTCTTCCAACCCAAT |
| | | factor 1 receptor | | |
| KPNA1 | SRP1, RCH2, NPI-1, | karyopherin alpha 1 | 3q21 | GCTTGGGCCATCACAAAT |
| | IPOA5 | (importin alpha 5) | | |
| KRAS | KRAS2 | v-Ki-ras2 Kirsten rat | 12p12.1 | TGGACGAATATGATCCAACAAT |
| | | sarcoma viral oncogene | | |
| | | homolog | | |
| LRIG1 | LIG-1, | leucine-rich repeats and | 3p14 | CCAGAATCACTGAAGGGTC |
| | DKFZP586O1624, | immunoglobulin-like | | |
| | LIG1 | domains 1 | | |
| MAP2 | MAP2A, MAP2B, | microtubule-associated | 2q34-q35 | AACCCTTTGAGAACACGAC |
| | MAP2C | protein 2 | | |
| MAPT | MTBT1, tau, PPND, | microtubule-associated | 17q21 | TGTGGCTCATTAGGCAAC |
| | FTDP-17, TAU, | protein tau | | |
| | MSTD, MIBT | | | |
| PPMID | Wip1 | protein phosphatase 1D | 17q233 | TTTCTGGCAGTAGCAAGAG |
| | | magnesium-dependent, | | |
| | | delta isoform | | |
| PTGS2 | | prostaglandin-endoperoxide | 1q25.2-q25.3 | GCTGAAGCCCTATGAATCATTT |
| | | synthase 2 (prostaglandin | | |
| | | G/H synthase and [symbol] | | |
| RABEP1 | | rabaptirt, RAB GTPase | 17p13.2 | CAGTGGAGAGAAGAAGTTGC |
| | | binding effector protein 1 | | |
| RARA | | retinoic acid receptor, | 17q12 | CAAAGCGCACCAGGAAAC |
| | | alpha | | |
| RHOC | | ras homolog gene family, | 1p13.1 | GCAGCCTGGGAACTTCAG |
| | | member C | | |
| ROPN1 | ODF6, ropporin, | ropporin, rhophilin | 3q21.1 | GAGTCGCTTTGTGTAACCG |
| | ROPN1A | associated protein 1 | | |
| S100A7 | | S100 calcium binding | 1q21 | TGCTGACGATGATGAAGGAG |
| | | protein A7 (psoriasin 1) | | |
| S100A8 | | S100 calcium binding | 1q12-q22 | CTGGAGAAAGCCTTGAACT |
| | | protein A8 (calgranulin A) | | |
| S100A9 | P14, MTF, NTF, | S100 calcium binding | 1q21 | GTGCGAAAAGATCTGCAAAA |
| | LIAG, MRP14, | protein A9 (calgranulin B) | | |
| | MAC387, 60B8AG | | | |
| SHC1 | SHC-P66 | SHC (fire homology 2 | 1q21 | GGGGTTTCCTACTTGGTTCG |
| | | domain containing) | | |
| | | transfonning protein 1 | | |
| TAP1 | | transporter 1, ATP-binding | 6p21.3 | GCCAGGAGACGGAGTTT |
| | | cassette, sub-family B | | |
| | | (MDR/TAP) | | |
| TP73L | | tumor protein p73L | 3q27-28) | CACTCTCCATGCCATCCAC |
| CKS2 | | CDC28 protein kinase | 9q22 | TGGAGGAGACTTGGTGT |
| | | regulatory subunit 2 | | |
| FAM54A | DUFD1 | family with sequence | 6q23.2 | GTGGAAATGCAGGAACTGAA |
| | | similarity 54, member A | | |
| GTPBP4 | CRPG, NGB, | GTP binding protein 4 | 10p15-p14 | GGATCATTACAAGTTGGCTCT |
| | FLJ10690, FLJ10686 | | | |
| HSPA14 | HSP70-4, HSP70LI | heat shock 70 kDa protein | 10p14 | TGGAATTGGACAAGACTCCC |
| | | 14 | | |
| PCNA | | proliferating cell nuclear | 20pter-p12 | CCACTCTCTTCAACGOT |
| | | antigen | | |
| FOXA1 | HNF3A | forkhead box A1 | 14q12-q13 | GCTACTACGCAGACACG |
| GATA3 | | GATA binding protein 3 | 10p15 | CATTAAGCCCAAGCGAAGG |
| CDCA1 | NUF2R | cell division cycle | 1q23.1 | GGAGGCGGAAGAAACCAG |
| | | associated 1 | | |
| AGR2 | XAG-2, HAG-2, AG2 | anterior gradient 2 | 7p21.3 | TTTGTCCTCCTCAATCTGGTTT |
| | | homolog (Xenopus laevis) | | |
| ESR1 | NR3A1, Era, ESR | estrogen receptor 1 | 6q24-q27 | GCAGGGAGAGGAGTTTGT |
| SCUBE2 | Cegf1, Cegb1, | signal peptide, CUB | 11p15.3 | GTTCCAGGTCCCATACG |
| | FLJ16792 | domain, EGF-like 2 | | |
| BUB1 | hBUB1, BUB1A, | BUB1 budding uninhibited | 2p11-q21 | GTTTGCGGTTCAGGTTTGG |
| | BUB1L | by benzimidazoles 1 | | |
| | | homolog (yeast) | | |
| SLC39A6 | LIV-1 | solute carrier family 39 | 18q12.2 | TCGAACTGAAGGCTATTTACGAG |
| | | (zinc transporter), | | |
| | | member 6 | | |
| UGT8 | CGT | UDP glycosyltransferase 8 | 4q26 | AACTCCGAAGCCTCCCTTA |
| | | (UDP-galactose ceramide | | |
| | | galactosyltransferase) | | |
| LOC400451 | | Hypothetical gene supported | 15q26.1 | CCAGGGTTTGTGTATTTGC |
| | | by AK075564, BC060873 | | |
| KNTC2 | HEC, HEC1 | kinetochore associated 2 | 18p11.31 | TGGGTCGTGTCAGGAAAC |
| TMC5 | FLJ13593 | transmembrane channel- | 16p13.11 | GCCTGGGTTGTCTCTACAGG |
| | | like 5 | | |
| ERBB2 | NEU ,HER-2 | v-erb-b2 crythroblastic | 17q11.2-q12 | GCTGGCTCTCACACTGATAG |
| | | leukemia viral oncogene | | |
| | | homolog 2, | | |
| | | neuro/glioblastoma | | |
| CA12 | HsT18816 | carbonic anhydrase XII | 15q22 | GCAGGTCCAGAAGTTCGATG |
| DKFZp762E1312 | | Hypothetical protein | 2q37.1 | GCTCCAAGGAGAACTTCATAC |
| | | DKFZp762E1312 | | |
| BIRC5 | EPR-1, API4 | baculoviral LAP repeat- | 17q25 | GCACAAAGCCATTCTAAGTC |
| | | containing 5 (survivin) | | |
| ANLN | ANILLIN, Scraps | anillin, actin binding | 7p15-p14 | ACAGCCACTTTCAGAAGCAAG |
| | | protein (scraps homolog, | | |
| | | Drosophila) | | |
| CEP55 | FLJ10540, CEP55, | centrosomal protein 55 kDa | 10q24.1 | CCTCACGAATTGCTGAACTT |
| | C10orf3 | | | |
| REEP6 | DPIL1, FLJ25383, | receptor accessory | 19p13.3 | CGAGTTCTTCAGCGATCTAC |
| | C19orf32 | protein 6 | | |
| ELOVL5 | HELO1, dJ483K16.1 | ELOVL family member 5, | 6p21.1-p12.1 | CCCTTCCATGCGTCCATA |
| | | elongation of long chain | | |
| | | fatty acids | | |
| | | (FEN1/Elo2, [symbol] | | |
| TMEM45B | LOC120224 | Transmembrane protein 45B | 11q24.3 | GTCGAAGCCGCAATTAGG |
| TTK | MPS1L1 | TTK protein kinase | 6q13-q21 | GGAGTTTGGGTTCCATCTT |
| AR | AKR1B1, ALDR1 | aldo-keto reductase family | 7q35 | TGTCCATCTTGTCGTCTTC |
| | | 1, member B1 (aldose | | |
| | | reductase) | | |
| CTSL2 | CTSU, CTSV | cathepsin L2 | 9q22.2 | GTACCAGTGGAAGGCAAC |
| CENPA | | cenromere protein A, 17 kDa | 2p24-p21 | CTGCACCCAGTGTTTCTGTC |
| GALNT7 | GALNAC-T7 | UDP-N-acetyl-alpha-D- | 4q31.I | GCACTGTGCCGCTTATAG |
| | | galactosamine:polypeptide | | |
| | | N- | | |
| | | acetylgalactosarminyltrans | | |
| DNAJC12 | JDP1, “J domain | DnaJ (Hsp40) homolog, | 10 | GAGTCGAGCCCGCTATGA |
| | protein 1” | subfamily C, member 12 | | |
| MLPH | 1Rk3, I(1)-3Rk, | melanaphilin | 2q37.2 | GTGGAATGCCTGCTGACC |
| | Slac-2a, In, | | | |
| | exophilin-3 | | | |
| TACSTD1 | Ly74, TROP1, GA733- | tumor-associated calcium | 2p21 | AGTTGGTGCACAAAATACTG |
| | 2, EGP, KSA M4S1, | signal transducer 1 | | TCAT |
| | MIC18 | | | |
| CDC20 | p55CDC | CDC20 cell division cycle | 1p34.1 | CTGTCTGAGTGCCGTGGAT |
| | | 20 homolog (S. cerevisiae) | | |
| PIP | | prolactin-induced protein | 7q32-qter | TGCCTATGTGACGACAATCC |
| CDCA7 | ELJ14736, JPO1 | cell division cycle | 2q31 | AAAGAGGAAGACCGTGGATGG |
| | | associated 7-variant2 | | |
| MIA | CD-RAP | melanoma inhibitory | 19q13.32-q13.3 | CCAGTAGCATTGTCCGAG |
| | | activity | | |
| XBP1 | XRP1 | X-box binding protein 1 | 5q22.2 | CTGGAACAGCAAGTGGTAG |
| C4orf34 | FLJ3289, LOC201895 | chromosome 4 open reading | 4p14 | TCAAGTAAAATCAAGCTGGG |
| | | frame 34 | | TAATC |
| VAV3 | | vav 3 oncogene | 1p13.3 | ACAAGGGACACTCAAACTAC |
| GRB7 | | growth factor receptor- | 17q11.2-17q21 | CGTGGCAGATGTGAACGA |
| | | bound protein 7 | | |
| UBE2C | UBCH10 | ubiquitin-conjugating | 20 | TGCCCTGTATGATGTCAGGA |
| | | enzyme E2C | | |
| PH-4 | | hypoxia-inducible factor | 3p21.31 | ACCGACAGGGATCACTTCAT |
| | | prolyl 4-hydroxylase | | |
| ART3 | | ADP-ribosyltransferase 3 | 4p14-p15.1 | TTGAACCCACCCAAATACCT |
| MELK | KIAA0175 | maternal embryonic leucine | 9p13.1 | CCAACAAAATATTCATGGTT |
| | | zipper kinase | | CTTG |
| CDCA8 | FLJ12042 | cell division cycle | 1p34.2 | TCCTTTCTGAAAGACTTCGACC |
| | | associated 8 | | |
| DNALI1 | P28 | dynein, axonemal, light | 1p35.1 | CCGCAGGGAACTCTACTCAC |
| | | intermediate polypeptide 1 | | |
| KIAA1370 | FLJ10980 | KIAA1370 | 15q21.2-q21.3 | ATGGATCTTGGAGCCAGTTC |
| THSD4 | FVSY9334, PRO34005, | thromhospondin, type 1, | 15q23 | GTGGGAACCATTTGCAGAAG |
| | FL313710 | domain containing 4 | | |
| KRT18 | | keratin 18 | 12q13 | TGATGACACCAATATCACACGA |
| MYO5C | | myosin VC | 15q21 | GGCCTACAGCCGAGGATT |
| FBP1 | FBP | fructose-1,6-bisphosphatase | 9q22.3 | GTGTCCGTTGGAACCAT |
| | | 1 | | |
| CDC45L | CDC45L2 | CDC45 cell division cycle | 22q11.2 | GTTTGAGCTGGCTTGGATG |
| | | 45-hire (S. cerevisiae) | | |
| CXXC5 | HSPC195 | CXXC finger 5 | 5q31.3 | CATGAAATAGTGCATAGTTT |
| | | | | GCC |
| FANCA | FACA, FANCH, FAA, | Fanconi anemia, | 16q24.3 | GCCATCATGGTGTTTGAG |
| | FA-H, FAH | complementation group A | | |
| MYB | c-myb | v-myb myeloblastosis viral | 6q22-q23 | GCTCCTAATGTCAACCGAGAA |
| | | oncogene homolog (avian) | | |
| OGFRLI | dJ331H24.1 | opioid growth factor | 6q13 | GAGCACAACCACACTTACATTC |
| | | receptor-like 1 | | |
| KIF2C | MCAK | kinesin family member 2C | 1p34.1 | GGAGATCCGTCAACTCCAAA |
| RRM2 | | ribonucleotide reductase | 2p25-p24 | CAGCAAGCGATGGCATAGT |
| | | M2 polypeptide | | |
| FOXC1 | FREAC3, ARA, IGDA, | forkhead box C1 | 6p25 | GATGTTCGAGTCACAGAGG |
| | IHG1 | | | |
| SFRP1 | SARP2, FRP, FRP1 | secreted frizzled-related | 8p12-p11.1 | AATGCCACCGAAGCCTC |
| | | protein 1 | | |
| AURKA | STK6 | serine/threomine kinase 6 | 20q13.2-20q13.3 | TCCAGGCCACTGAATAACAC |
| ACTR3B | ARP11, ARP3beta, | ARP3 actin-related protein | 7q34 | AAAGATTCCTGGGACCTGA |
| | ARP3BETA | 3 homolog B (yeast) | | |
| TCF7L1 | TCF3 | transcription factor | 2p11.2 | CCATGAACGCCTCGATGT |
| | | 7-like 1 (T-cell specific, | | |
| | | HMG-box) | | |
| MYBL2 | BMYB | v-myb myeloblastosis | 20q13.1 | CGAGATCGCCAAGATGTT |
| | | viral oncogene homolog | | |
| | | (avian)-like 2 | | |
| CELSR1 | ME2, HFMI2, FMI2, | cadherin, EGF LAG seven- | 22q13.1 | TGGTGACAGTGGATGATTGTG |
| | CDHF9 | pass G-type receptor 1 | | |
| | | (flamingo homolog, [symbol] | | |
| NTN4 | | netrin 4 | 12q22-12q23 | CCAGGCTTCTATCGTGAC |
| SLC16A6 | MCT6 | solute carrier family 16 | 17q24.3 | TGGATAATCTCAATCTGTGTG |
| | | (monocarboxylic acid | | TTTG |
| | | transporters), member 6 | | |
| C10orf38 | FLJ12884 | chromosome 10 open reading | 10p13 | GTGGCGGTTTGACCAGAA |
| | | frame 38 | | |
| GPR160 | GPCR150, GPCR1 | G protein-coupled receptor | 3q26.2-q27 | TTCGGCTGGAAGGAACC |
| | | 160 | | |
| TFF3 | HITF | trefoil factor 3 | 21q22.3 | TGCTGGGGCTGGTCCTG |
| | | (intestinal) | | |
| PIB5PA | | phosphatidylinositol (4,5) | 22 | AACTTCGCTCCCACCTTC |
| | | bisphosphate 5-phosphatase, | | |
| | | A | | |
| BCL11A | Evi9, BCL11A-XL, | B-cell CLL/lymphoma 11A | 2p16 .l | CCCAAACAGGAACACATAGCA |
| | BCL11A-L, BCL11A-S, | (zinc fmger protein) | | |
| | EVI9 | | | |
| E2F1 | RBP3 | E2F transcription factor 1 | 20q11-20q11 | AGACCGTAGGTGCGATCAG |
| RACGAP1 | MgcRacGAP | Ran GTPase activating | 12q13 | GCCTTAACAGAGCCTITATGGA |
| | | protein 1 | | |
| TRIP13 | | thyroid hormone receptor | 5p15 | CTCATGCGCTGTATGTCCA |
| | | interactor 13 | | |
| UBE2T | HSPC150 | ubiquitin-conjugating | 1q32.1 | GTGAGGGGTGTCAGCTCAGT |
| | | enzyme E2T (putative) | | |
| CAPN13 | FLJ23523 | calpain 13 | 2p22-p21 | TTCCACTCGATTTCCAAGTGA |
| ACOT4 | ACOT4, FLJ31235, | acyl-CoA thioesterase 4 | 14q24.1 | GTATGCTACATGTTCAACATCC |
| | PTE-Ib, | | | |
| PRC1 | | protein regulator of | 15q26 | ACCATTATGTCTGGGTCAAAGG |
| | | cytokinesis 1 | | |
| SPDEF | PDEF, bA375E1.3 | SAM pointed domain | 6p21.3 | CTGCAAGCTGCTCAACATC |
| | | containing ets | | |
| | | transcription factor | | |
| NAT1 | | N-acetyltransferase 1 | 8p23.1-p21.3 | AGCCTCGAACAATTGAAGA |
| | | (arylamine | | |
| | | N-acetyltransferase) | | |
| KIAA1324 | maba1 | KIAA1324 | 1p133 | TTCCTACTCCAATGGCTCAGA |
| TSPAN13 | NET-6, TM4SF13, | tetsaspanin 13 | 7p21.2 | GCCATGTGCTCCAATCATAG |
| | TSPAN13 | | | |
| MAD2L1 | MAD2, HSMAD2 | MAD2 matotic arrest | 4q27 | GGTGACATTTCTGCCACTG |
| | | deficient-like 1 (yeast) | | |
| NEK2 | NLK1, “HsPK 21” | NIMA (never ln mitosis gene | 1q32-q42 | ACATTTGTTGGCACACCTTA |
| | | a)-related kinase 2 | | |
| NPDC1 | | neural proliferation, | 9q34.3 | GCTCTGTGTGCCCAGGAT |
| | | differentiation and | | |
| | | control, 1 | | |
| GPSM2 | LGN, Pins | G-protein signalling | 1p13.3 | ATTGACCACCGAATTCCAAA |
| | | modulator 2 (AGS3-like, | | |
| | | C. elegans) | | |
| DLG7 | KIAA0008, DLG1, | discs large homolog 7 | 14q22.1-q22.3 | AAATGCCGGTCCTCAGAATAC |
| | HURP | (Drosophila) | | |
| SLC40A1 | MTP1, IREG1, FPNI, | solute carrier family 40 | 2q32 | GATTGTTGTTGTTGCAGGAGA |
| | HFE4 | (iron-regulated | | |
| | | transporter), member 1 | | |
| ORC6L | ORC6 | origin recognition complex, | 16q12 | ATCGACTGTGTAAACAACTAG |
| | | subunit 6 homolog-like | | AGAAGA |
| | | (yeast) | | |
| BCMP11 | HAG3, hAG-3 | breast cancer membrane | 7p21.1 | TGAAGAAGGTCTCTTTTATCT |
| | | protein 11 | | CTCA |
| EXO1 | HEX1, hExoI | exonuclease 1 | 1q42-q43 | CCCATCCATGTGAGGAAGTATAA |
| KIF20A | RAB6KIFL | kinesin family member 20A | 5q31 | AAGCCACACACAGGTTC |
| EPN3 | FLJ20778 | epsin 3 | 17q21.33 | CACCTTCGCTTCCAGATG |
| PTTG1 | PTTG, HPTTG, EAP1, | pituitary tumor- | 5q35.1 | CCTCAGATGATGCCTATCCA |
| | securin | transforming 1 | | |
| RERG | MGC15754 | RAS-like, estrogen- | 12p13.1 | AACTCGCAAACGCAACCT |
| | | regulated, growth inhibitor | | |
| TMEM25 | FLJ14399, | transmembrane protein 25 | 11q23.3 | CAAGGTTTCATCCGCCTC |
| | 0610039I01Rik | | | |
| PHGDH | SERA, PGDH, PDG | phosphoglycerate | 1p12 | TGCCGCAGAACTCACTTG |
| | | dehydrogenase | | |
| SLC9A3R1 | NHE3 | solute carrier family 9 | 5p153 | CCAATGGGGAGATACAGAAGG |
| | | (sodium/hydrogen | | |
| | | exchanger), member 3 | | |
| FAM64A | FLJ10156 | family with sequence | 17p132 | CCATTACGGCGATCAAGG |
| | | similarity 64, member A | | |
| SEMA3C | SemE\SEMAE | Sema domain, immunoglobulin | 7q21-q31 | GACAAAGACAGGAGGAAAGAG |
| | | domain (Ig), short basic | | |
| | | domain, secreted, (sema [symbol] | | |
| PGR | PR, NR3C3 | progesterone receptor | 11q22-q23 | TTTAAGAGGGCAATGGAAGG |
| BCL2 | Bcl-2 | B-cell CLI/lymphoma 2 | 18q21.3 | TACCTGAACCGGCACCTG |
| ABCC3 | MRP3, cMOAT2, | ATP-binding cassette, | 17q21 | TGCTCTCCTTCATCAATCCA |
| | EST90757, | sub-family C (CFIRflaP), | | |
| | MLP2, MOAT-D | member 3 | | |
| CCND1 | BCL1, D11S287E, | cyclin D1 | | GAAGATCGTCGCCACCTG |
| | PRAD1 | | | |
| CCNE1 | CCNE | eyclin E1 | 19q12 | GGCCAAAATCGACAGGAC |
| CDH1 | uvomorulin | cadherin 1, type 1, | 16q22.1 | CCACCAAAGTCACGCTGAA |
| | | E-cadherin (epithelial) | | |
| EGFR | ERBB | epidermal growth factor | 7p12 | ACACAGAATCTATACCCACC |
| | | receptor (erythroblastic | | AGAGT |
| | | leukemia viral | | |
| | | (v-orb-b) [symbol] | | |
| KRT6B | KRTL1 | keratin 6B | 12q12-q13 | TCGACCACGTCAAGAAGC |
| MYC | c-Myc | v-myc myelocytomatosis | 8q24 | AGGCGAACACACAACGTC |
| | | viral oncogene homolog | | |
| | | (avian) | | |
| KRT5 | | keratin 5 (epidermolysis | 12q | GTTGGACCAGTCAACATCTCTG |
| | | bullosa simplex, | | |
| | | Dowling-Meara/Kobner/W | | |
| | | eber-Cock [symbol] | | |
| GSTP1 | | glutathione S-transferase | 11q13-gter | ACCTCACCCTGTACCAGTC |
| | | pi | | |
| B3GNT5 | B3GN-T5, | UDP-GlcNAc:betaGal beta- | 3q28 | CCGGAGCTGCCTATGTAATC |
| | beta3Cm-T5 | 1,3-N-acetylglucosaminyl- | | |
| | | transferase 5 | | |
| COX6c | | cytochrome c oxidase | 8q22-q23 | CATTCGTGCTATCCCTGG |
| | | subunit VIc | | |
| FZD7 | FzE3 | frizzled homolog 7 | 2q33 | CTGACCCTGTCTCTGTGT |
| | | (Drosophila) | | |
| TCEAL1 | p21, pp21, SIIR, | transcription elongation | Xq22.1 | CAACATGGACAAACCACG |
| | P21 | factor A (S11)-like 1 | | |
| KIT | PBT, CD117, SCFR, | v-kit Hardy-Zuckerman 4 | 4q11-q12 | ATTCCCAGAGCCCACAATA |
| | C-Kit | feline sarcoma | | |
| | | viral oncogene homoloc | | |
| RRT17 | PCHC1 | keratin 17 | 17q12-17q21 | ACTCAGTACAAGAAAGAACCG |
| CDH3 | CDHP, PCAD | cadherin 3, type 1, | 16q22.1 | GACAAGGAGAATCAAAAGATC |
| | | P-cadherin (placental) | | AGC |
| GSTM3 | GST5 | glutathione S-transferase | 1p133 | CAAGCTAGACCTGGACT |
| | | M3 (brain) | | |
| TP53BP2 | 2 choices: | TP53BP1 tumor protein p53 binding | | AGGCTCTGCTTCTGTACC |
| | PPP1R13A, | protein, 1 (15q15-q21) or TP53BP2 | | |
| | TP53BP | tumor protein p53 bindi [symbol] | | |
| CENPF | | centromere protein P, | 1q32-q41 | GTGGCAGCAGATCACAA |
| | | 350/400 ka (mitosin) | | |
| TOP2A | | topoisomerase (DNA) II | 17q21-q22 | CAACATGCCAATTGAGTGAAA |
| | | alpha 170 kDa | | |
| Tvms | | thymidylate synthetase | 18p1131-p11.21 | CAAACGTGTGTTCTGGAAGG |
| CCNB1 | CCNB | cyclin B1 | 5q12 | CTTTCGCCTGAGCCTATTT |
| MKI67 | | antigen identified by | 10q25-qter | GTCTCTGGTAATGCACACT |
| | | monoclonal antibody Ki-67 | | |
| CLDN3 | RVP1, C7orf1, | claudin 3 | 7q11 | CTACGACCGCAAGGACTACG |
| | CPETR2 | | | |
| CLDN4 | CPE-R, WBSCR8, | claudin 4 | 7q11.23 | ATCGGCAGCAACATTGTCA |
| | hCPE-R, CPETR, | | | |
| | CPETR1 | | | |
| CRYAB | HSPB5 | crystallin, alpha B | 11q22.3-q23.1 | CAAGGAAACAGGTCTCTGG |
| CTPS | | CTP synthase | 1p34.1 | TGCCATGTTGAGCCTGA |
| GSDML | PRO2521 | gasdarmin-like | 17q21.2 | TGGATTCTGGGCTCCAAG |
| KRT14 | EBS3, EBS4 | keratin 14 (epidermolysis | | |
| | | bullosa simplex, Dowling- | | |
| | | Meara, koebner) | | |
| KRT19 | | keratin 19 | 17q21-q23 | GTCATGGCCGAGCAGAAC |
| KRT8 | | keratin 8 | 12q13 | GATGAACCGGAACATCAGC |
| RARRESS | | retinoic acid receptor | 11q23 | AGCACTTTGTCACCCAG |
| | | responder (tazarotene | | |
| | | induced) 3 | | |
| TRIM29 | ATDC | tripartite motif-containing | 11q22-q23 | TGAGATTGAGGATGAAGCTGAG |
| | | 29-Variant 1 | | |
| VEGF | | vascular endothelial | 6p21-p12 | AGTGTGTGCCCACTGAGGA |
| | | growth factor | | |
| WIRE | WICH | WIRE protein | 17q212 | CAACATTAATGATCGGAGTGCT |
| YBX1 | NSEP1 | Y box binding protein 1 | 1p34 | CAGTATTCCAACCCTCCTGTG |

| Reverse Sequence | | |
|---|---|---|
| (SEQ ID NO: 242-454) | Source | |
| CAGGTCTTTGCGGATGTC | Housekeeper | corresponds to gene from 1st page |
| | | of table 21 and is respectively in |
| | | order of those genes |
| GGCCATCCACAGTGTTCT | Housekeeper | |
| CCGTAGTCGTGATACCAAGA | Housekeeper | |
| GACAGACACTGGCAACA | Housekeeper | |
| TTACAATAGCCCAAGTAGCCA | Housekeeper | |
| TCCTGGACCCGAGGATTAT | Housekeeper | |
| GCCCTCACCCAGATACT | Housekeeper | |
| TGGCACGCTCCAGTTTC | Housekeeper | |
| GCTCAGCCAACTGCTTC | Housekeeper | |
| CCACGAAGAACCAGTAGC | MIP2 | |
| AGGGCTGGAGATTTTTGC | MIP2/MIP3 | |
| TTGTGACCCTGGGATTAGATG | MIP3 | |
| TTCCTCCATCAAGAGTTCAACA | MIP3 | |
| GGGTGCCACATTOGAAGA | MIP3 | |
| AGATGTCGCTGAGGGTT | MIP3 | |
| GATGGCGACCAAGTTCT | MIP3 | |
| TCTTTGGGGGAGTTAGACAC | MIP3 | |
| GTGATGTAGAAGAATCCACCATAG | MIP3 | |
| TTCATCTCTCCAATCTCACCAG | MIP3 | |
| CAGGAACATTTTTATGCCTTCTC | MIP3 | |
| TGTCTCTGCTGCTGTCC | MIP3 | |
| AGGTCCAGGATGTAGTCG | MIP3 | |
| TGGTGAGGGCTCCTTGA | MIP3 | |
| GCGGTTCATTTGGAGTCT | MIP3 | |
| TCAGGGAACCAAGGTCG | MIP3 | |
| TCCGCTTCTCCTGCTCA | MIP3 | |
| CGGGACACATTGATCTTTACA | MIP3 | |
| GCAAATCCTTGGGCAGA | Other | |
| AGGGCAGATTGGGAAAG | Other | |
| TCACAGGGTCAAACTTCCAGT | Other | |
| CTGGAGCCTCAGGGAGA | Other | |
| TATTACCGAGGCGAAGAGT | Other | |
| CCTCTCAGCCTCTCTGTG | Other | |
| GAGGATCCTTGGGTTATAATTGG | Other | |
| TGTAGAAGAGATGACACTCGG | Other | |
| CAGACATGTTGGTATTGCACATT | Other | |
| TCCAGATGATTCTTTAACAGGTAGC | Other | |
| CCCTTTTTGGACTTCAGGTG | Other | |
| ACTTGGGATATGTGAATAAGACC | Other | |
| GGATTCAAGTAGGATGCTGC | Other | |
| ACTGCTGGGTTTGTGTAAG | Other | |
| CGTCGATGCCTATGACAGC | Other | |
| ATACCAGGAGCAAGCIACC | Other | |
| GGGTCCTCATCATCGTTG | Other | |
| GGCTTTCCCTTTCCCAT | Other | |
| TGGCTTTGGTCGTTCTGTTT | Other | |
| CAGTCACAGATGGTTTTGCAC | Other | |
| CCAAGCATGACTTCAGATTC | Other | |
| CTCAGCATCACTGTCTGGTTA | Other | |
| AGTAGAACTCGGGCAAGCTG | Other | |
| CAGGAGTGGGCGTTTTCTT | Other | |
| CTCGCAACGATCTTCGTC | Other | |
| GAGGACATGGTGTGCATC | Other | |
| CGGCTTGATACAACCCAGTT | Other | |
| TCCCTCATTGCACTGTACTCC | Other | |
| AGGAAGTCATCGCACAC | Other | |
| TCTTTCCGTTCATCTGCCA | Other | |
| CTTCGACTGGACTCTGT | Other | |
| ACTTGTGTCTGGTTCAGG | Other | |
| TCCAACTCTGCAGACATTTCC | Other | |
| CTGGTGCTCATAGTCACG | Other | |
| GTTGTTCTGAGCTGTTGTTCGT | Other | |
| CACCAGCTTCTTTCGGATTG | Other | |
| TGAGAATGCAGGATCTTTAACAG | Other | |
| CGAGGTAATTTGTGCCCTTT | Other | |
| CTGTAGACGGCATGGAAAT | Other | |
| TCAGCTGCTTGTCTGCATTT | Other | |
| CCGGGTGTTGAAGTCCAG | Other | |
| CGTGTCCTCTGTTACCCGA | Other | |
| GCCCAACCTCGCTAAGAAA | Other | |
| GAATATGTGGTTCTGGCTCA | Proliferation | |
| GCTCGTCACTCAAGCCAA | Proliferation | |
| CTTCATCAGTCGCACATAATCT | Proliferation | |
| ACGCTGAGAGATAAGGATG | Proliferation | |
| AGTGTCCCATATCCGCA | Proliferation | |
| CTGAGTTCATGTTGCTGACC | Top 100 (1) | |
| TGACAGTTCGCACAGGAC | Top 100 (10) | |
| GGGGAAAGACAAAGTTTCCA | Top 100 (100) | |
| CATAATCCTGGGGACATACTGG | Top 100 (11) | |
| GACTTCAGGGTGCTGGAC | Top 100 (12) | |
| TAGAGCCTGCCATCTCG | Top 100 (13) | |
| CATGTGGGCTTCAAGCATC | Top 100 (14) | |
| CTGCTGAGAATCAAAGTGGGA | Top 100 (15) | |
| GTGTTTGTGCGCTGAATC | Top 100 (16) | |
| ACTGAAGAACCGAAGATGG | Top 100 (17) | |
| CACCGCTGGAAACTGAAC | Top 100 (18) | |
| CCCCAGGGTTACTGTGTGTC | Top 100 (19) | |
| GCCCTTACACATCGGAGAAC | Top 100 (2) | |
| CCGCAGTACAGACTTGCACTT | Top 100 (20) | |
| CTTGCAATCFCTTAATGCCC | Top 100 (21) | |
| GACGCTTCCTATCACTCTATTC | Top 100 (22) | |
| CGATGGTTTTGTACAAGATTTCTC | Top 100 (23) | |
| CCACAGTCTGTGATAAACGG | Top 100 (24) | |
| AGCCATGCAGAACAACAG | Top 100 (25) | |
| TGTCAGCACAAACTGAAGCA | Top 100 (27) | |
| GGAACAAACTGCTCTGCCA | Top 100 (28) | |
| TTCTCTGCCACTTAAATCCTCG | Top 100 (29) | |
| CTCCTTCCTCCTGTAGITTC | Top 100 (3) | |
| ACACTGCTCTCCTCCATC | Top 100 (30) | |
| GAGAGTCCCCGGTATCATCC | Top 100 (31) | |
| TCGGGCATACCCATCTTC | Top 100 (32) | |
| CAACCCAGTGCATTGACG | Top 100 (33) | |
| CGCACTCCAGCACCTAGAC | Top 100 (34) | |
| TCCCAAGTTTTGAGCCATTC | Top 100 (35) | |
| TCCTTGTAATGGGGAGACCA | Top 100 (36) | |
| GGCTGCAATTTGCACAGTTC | Top 100 (37) | |
| CACTGGGCGAATTATATGCG | Top 100 (38) | |
| CCCATTTGTCTGTCTTCAC | Top 100 (39) | |
| GCCATGAGTTTTCTCTCGT | Top 100 (4) | |
| TAGGACTGGGACTGCCGTAA | Top 100 (40) | |
| TGTTTAGGAGTTCTTCGCAG | Top 100 (41) | |
| AGTGGGCATCCCGTAGA | Top 100 (42) | |
| GGGACTATCAATGTTGGGTTCTC | Top 100 (43) | |
| AGCCGACACTCTTCATCAGTC | Top 100 (44) | |
| GATGCAGAAGGATGGCTTTT | Top 100 (45) | |
| AGGCGATCCTGGGAAATTAT | Top 100 (46) | |
| CCTGTCTGACTCAATTTGCT | Top 100 (47) | |
| GGATCTCGTCCCGGACTC | Top 100 (48) | |
| ACACAAATGAGCGGACAG | Top 100 (49) | |
| ATTGCCTGGCAGTTCAACTC | Top 100 (5) | |
| GGCTTGTAGGCCTTTTACTTCC | Top 100 (51) | |
| GCCTTATGTTCCTCCAGCAT | Top 100 (52) | |
| CTCAGAAGGCTCATCAGT | Top 100 (53) | |
| TCTTGTCTTGCACCCACTG | Top 100 (54) | |
| CCATCAACATTCTCTTTATGAACG | Top 100 (55) | |
| GAAGTGGGACACGTAGTAAG | Top 100 (56) | |
| AGCTGCATGTGTGGTTCTGT | Top 100 (57) | |
| GAAGTTCAAGCCTTGTTCTC | Top 100 (58) | |
| AGTGGACATGCGAGTGGAG | Top 100 (59) | |
| AGCGGGCTTCTGTAATCTGA | Top 100 (59) | |
| GACAGCTACTATTCCCGTT | Top 100 (6) | |
| GCCTCAGATTTCAACTCGT | Top 100 (60) | |
| TTTGATGCCAGTECCTCCTC | Top 100 (61) | |
| TGGGGCAGTTCTGTATTACTTC | Top 100 (62) | |
| GAGCCACCATGTGAGGAGAG | Top 100 (63) | |
| GATGGTAGAGTTCCAGTGATT | Top 100 (64) | |
| CGGTCAGATCCAGGGACTT | Top 100 (64) | |
| AGTTGGCAGGAAGGACA | Top 100 (66) | |
| CGAAACGATTGCTCAGGACT | Top 100 (67) | |
| TGGTGCACAAGACCCAGAC | Top 100 (68) | |
| TATGTGAGTAAGCTCGGAGAC | Top 100 (69) | |
| GGCACGGCACACTGGTT | Top 100 (7) | |
| GCTGGCTTCCGTTTCTTG | Top 100 (70) | |
| GAGCTCCATGTGCAGAACG | Top 100 (71) | |
| GGTGGTGGTGACACTATGG | Top 100 (72) | |
| CAGCTATGCTGTTGTciICA | Top 100 (73) | |
| GTCCACTGCCAGAGACAGG | Top 100 (74) | |
| CACACAGTTCACTGCTCCACA | Top 100 (75) | |
| GTGGAAATTTCTCCCGGAAC | Top 100 (76) | |
| AGGCCATTGAGAGACAAATATC | Top 100 (77) | |
| TTCTTCCAACCGATCCACTTC | Top 100 (78) | |
| CGGTATTGGTGCTCTGTC | Top 100 (79) | |
| ACACAGATGATGGAGATGTC | Top 100 (8) | |
| AGCGTGTTCCACCATTTGTA | Top 100 (80) | |
| GCCAAACACCCAGGATCTC | Top 100 (82) | |
| GTCCCGACTCTTCCCAT | Top 100 (83) | |
| ATTGTAGGACATGCGATTCA | Top 100 (84) | |
| GGAAGTCAATCTCATCTECCAGTC | Top 100 (85) | |
| CAAAGAACCCTTCATCTCCAA | Top 100 (86) | |
| TCCTGCTTTCAGGAATACTC | Top 100 (87) | |
| CCTTCGTATTGTGGCATTC | Top 100 (88) | |
| AGTAGCTACATCTCCAGGTTCTCTG | Top 100 (89) | |
| TGGGCAAATACTTTCTTTAGTGC | Top 100 (9) | |
| TGTGAAGCCAGCAATATGTATC | Top 100 (90) | |
| CATCTCCTTCACAGTTAGGTEG | Top 100 (91) | |
| GCCTATEGTCTCTTGCTGTE | Top 100 (92) | |
| GCAGGTCAAAACTCTCAAAG | Top 100 (93) | |
| TCTTGGAAGAGTCCACAATCC | Top 100 (94) | |
| TCATCACTGCTCACGCT | Top 100 (95) | |
| CATTTGCCGTCCTTCATCG | Top 100 (96) | |
| CACTGGAGGCGGATCTCA | Top 100 (97) | |
| CCCACAGGCTCTAGGTCACT | Top 100 (98) | |
| TCCCTGTGAAGTGGCTATTA | Top 100 (99) | |
| CGGATTTTATCAACGATGCAG | Top 141 | |
| GCCGTACAGTTCCACAAAGG | Top 141 | |
| TGGGGTTGGAGATAAACCTG | Top 141 | |
| GACCTCCTCCTCGCACTTCT | Top 141 | |
| GGGTCTGCACAGACTGCAT | Top 141 | |
| TGCTTGGATTCCAGAAACG | Top 141 | |
| ATCAACTCCCAAACGGTCAC | Top 141 | |
| GTTCTTAGCATCCTTGAGGG | Top 141 | |
| TCTGGTCACGCAGGGCAA | Top 141 | |
| GCCATAGCCACTGCCACT | Top 141 | |
| CTGCTGGTCCTTCCCATAG | Top 141 | |
| CAGAGGCCCATGAACACATC | Top 141 | |
| TGTAGAAATCTGCGTATGCC | Top 141 | |
| GTTCAAACCTTCCTCTTCGT | Top 141 | |
| CCTCTCCTCATCGGTCT | Top 141 | |
| ATCCACTGGCAGTACAGAAG | Top 141 | |
| GAGGAGATGACCTTGCC | Top 141 | |
| ACTGTCTGGGTCCATGGCTA | Top 141 | |
| GCATTGCTCTGGGTGAT | Top 141 | |
| CGGACGCACTTTCTTCTC | Top 141 | |
| GGATTTCGTGGTGGGTTC | Top 141 | |
| ACTTGGGCCTTAAACTTCACC | Top 141 | |
| ACAGCTCTTTAGCATTTGTGGA | Top 141 | |
| GGGCACATCCAGATGTTT | Top 141 | |
| CTGATGGTTGAGGCTGTT | Top 141 | |
| GTGGTGGTGTTGGTGGTG | Top 141 | |
| CACGCAGTTCATCCATAGG | Top 141 | |
| GCAGGCTTCTCTTCACG | Top 141 | |
| CAAGGGGACTCGGTAGA | Top 141 | |
| CAACTCTCCCGTTGAGTC | Top 141 | |
| CGTGCACATCCATGACCTT | Top 141 | |
| CCGGTTCAATTCTTCAGTCC | Top 141 | |
| CTCCAGGGAAGCCCTCTG | Top 141 | |
| GCCACACCAACTTCAACC | Top 141 | |
| CATTGGTGGTGAAGCTCTTG | Top 141 | |
| GGTGAGGTTTGATCCGCATA | Top 141 | |
| CTCCTCCAGAGCCATAGCC | Top 141 | |
| GTTCTCCTGCACCCTGTT | Top 141 | |

[symbol]

indicates data missing or illegible when filed

### G. REFERENCES

Aldlesh S, Shaffer D J, Roopenian D. “Customized molecular phenotyping by quantitative gene expression and pattern recognition analysis” Genome Res 13:1719-1727 (2003).
Bair, E., and Tibshirani, R. “Semi-supervised methods to predict patient survival from gene expression data” PLoS Biol 2:E108 (2004).
Bloom, H. J. G., and Richardson, W. W. “Histologic grading and prognosis in breast cancer” British Journal of Cancer 9:359-377 (1957).
Benito, M., Parker, J., Du, Q., Wu, J., Xiang, D., Perou, C. M., and Marron, J. S. “Adjustment of systematic microarray data biases” Bioinformatics 20:105-114 (2004).
Bhatia P, Taylor W R, Greenberg A H, Wright J A. “Comparison of glyceraldehyde-3-phosphate dehydrogenase and 28S-ribosomal RNA gene expression as RNA loading controls for northern blot analysis of cell lines of varying malignant potential” Anal Biochem 216:223-226 (1994).
Bullinger, L., Dohner, K., Bair, E., Frohling, S., Schlenk, R. F., Tibshirani, R., Dohner, H., and Pollack, J. R. “Use of gene-expression profiling to identify prognostic subclasses in adult acute myeloid leukemia” N Engl J Med 350:1605-1616 (2004).
Buzdar, A., O'Shaughnessy, J. A., Booser, D. J., Pippen, J. E., Jr., Jones, S. E., Munster, P. N., Peterson, P., Melemed, A. S., Winer, E., and Hudis, C. “Phase II, randomized, double-blind study of two dose levels of arzoxifene in patients with locally advanced or metastatic breast cancer” J Clin Oncol 21:1007-1014 (2003).
Caly, M., Genin, P., Ghuzlan, A. A., Elie, C., Freneaux, P., Klijanienko, J., Rosty, C., Sigal-Zafrani, B., Vincent-Salomon, A., Douggaz, A., et al. “Analysis of correlation between mitotic index, MIB1 score and S-phase fraction as proliferation markers in invasive breast carcinoma. Methodological aspects and prognostic value in a series of 257 cases” Anticancer Res 24:3283-3288 (2004).
Chia, S. K., Speers, C. H., Bryce, C. J., Hayes, M. M., and Olivotto, I. A. “Ten-year outcomes in a population-based cohort of node-negative, lymphatic, and vascular invasion-negative early breast cancers without adjuvant systemic therapies” J Clin Oncol 22:1630-1637 (2004).
Clark G. M., Allred, D. C., Hilsenbeck, S. G., Chamness, G. C., Osborne, C. K., Jones, D., and Lee, W. H. “Mitosin (a new proliferation marker) correlates with clinical outcome in node-negative breast cancer” Cancer Res 57:5505-5508 (1997).
Cronin, M., Pho, M., Dutta, D., Stephans, Shak, S., Kiefer, M. C., Esteban, S. M., and Baker, J. B. “Measurement of gene expression in archival paraffin-embedded tissues: development and performance of a 92-gene reverse transcriptase-polymerase chain reaction assay” Am J Pathol 164:35-42 (2004).
Dalton, L. W., Page, D. L., and Dupont, W. D. “Histologic grading of breast carcinoma. A reproducibility study” Cancer 73:2765-2770 (1994).
Dhanasekaran S M, Barrette T R, Ghosh D, Shah R, Varambally S, Kurachi K, Pienta K J, Rubin M A, Chinnaiyan A M. “Delineation of prognostic biomarkers in prostate cancer” Nature 412:822-826 (2001).
Diehn, M., Sherlock, G., Binkley, G., Jin, H., Matese, J. C., Hernandez-Boussard, T., Rees, C. A., Cherry, J. M., Botstein, D., Brown, P. O., et al. “SOURCE: a unified genomic resource of functional annotations, ontologies, and gene expression data” Nucleic Acids Res 31:219-223 (2003).
Dudoit, S., and Fridlyand, I. “A prediction-based resampling method for estimating the number of clusters in a dataset” Genome Biol 3:RESEARCH0036 (2002).
Efron, B., Tibshirani, R. J. “An Introduction to the Bootstrap” Boca Raton, Fla.: CRC Press LLC. p 247 pp (1998).
Eggert A, Brodeur G M, Ikegaki N. “Relative quantitative RT-PCR protocol for TrkB expression in neuroblastoma using GAPD as an internal control” Biotechniques 28:681-682, 686, 688-691 (2000).
Eisen, M. B., Spellman, P. T., Brown, P. O., and Botstein, D. “Cluster analysis and display of genome-wide expression patterns” Proc Natl Acad Sci USA 95:14863-14868 (1998).
Elston, C. W., and Ellis, I. O. “Pathological prognostic factors in breast cancer. L The value of histological grade in breast cancer: experience from a large study with long-term follow-up” Histopathology 19:403-410 (1991).
Fisher, E R., Osborne, C. K., McGuire, W. L., Redmond, C., Knight, W. A., 3rd, Fisher, B., Bannayan, G., Walder, A., Gregory, E. J., Jacobsen, A., et al. “Correlation of primary breast cancer histopathology and estrogen receptor content” Breast Cancer Res Treat 1:37-41(1981).
Fisher, B., Costantino, I., Redmond, C., Poisson, R., Bowman, D., Couture, J., Dimitrov, N. V., Wolmark, N., Wickerham, D. L., Fisher, E. R., et al. “A randomized clinical trial evaluating tamoxifen in the treatment of patients with node-negative breast cancer who have estrogen-receptor-positive tumors” N Engl I Med 320:479-484 (1989).
Fitzgibbons, P. L., Page, D. L., Weaver, D., Thor, A. D., Allred, D. C., Clark, G. M., Ruby, S. G., O'Malley, F., Simpson, J. F., Connolly, J. L., et al. “Prognostic factors in breast cancer. College of American Pathologists Consensus Statement 1999” Arch Pathol Lab Med 124:966-978 (2000).
Frank S G, Bernard, P. S. “Profiling Breast Cancer using Real-Time Quantitative PCR. In Rapid Cycle Real-Time PCR: Methods and Applications” Edited by S. Meuer W, C., Nakagawara, K. Heidelberg, Germany, Springer pp 95-106 (2003).
Frierson, H. F., Jr., Wolber, R. A., Berean, K. W., Franquemont, D. W., Gaffey, M. J., Boyd, I C., and Wilbur, D. C. “Interobserver reproducibility of the Nottingham modification of the Bloom and Richardson histologic grading scheme for infiltrating ductal carcinoma” Am J Clin Pathol 103:195-198 (1995).
Genestie, C., Zafrani, B., Asselain, B., Fourquet, A., Rozan, S., Validire, P., Vincent-Salomon,
A., and Sastre-Garau, X. “Comparison of the prognostic value of Scarff-Bloom-Richardson and Nottingham histological grades in a series of 825 cases of breast cancer: major importance of the mitotic count as a component of both grading systems” Anticancer Res 18:571-576 (1998).
Greenough, R. B. “Varying degrees of malignancy in cancer of the breast” J Cancer Res 9:452-463 (1925).
Gruvberger, S., Ringner, M., Chen, Y., Panavally, S., Saal, L. H., Borg, A., Ferno, M., Peterson, C., and Meltzer, P. S. “Estrogen receptor status in breast cancer is associated with remarkably distinct gene expression patterns” Cancer Res 61:5979-5984 (2001).
Henson, D. E., Ries, L., Freedman, L. S., and Carriaga, M. “Relationship among outcome, stage of disease, and histologic grade for 22,616 cases of breast cancer. The basis for a prognostic index” Cancer 68:2142-2149 (1991),
Ishida, S., Huang E., Zuzan, H., Spang, R., Leone, G., West, M., and Nevins, J. R. “Role for E2F in control of both DNA replication and mitotic functions as revealed from DNA microarray analysis” Mol Cell Biol 21:4684-4699 (2001).
Iwahashi, H., Eguchi, Y., Yasuhara, N., Hanafusa, T., Matsuzawa, Y., and Tsujimoto, Y. “Synergistic anti-apoptotic activity between Bcl-2 and SMN implicated in spinal muscular atrophy” Nature 390:413-417 (1997).
Kollias, J., Murphy, C. A., Elston, C. W., Ellis, I. O., Robertson, J. F., and Blarney, R. W. “The prognosis of small primary breast cancers” Eur J Cancer 35:908-912 (1999).
Kristt D, Turner I, Koren R, Ramadan E, Gal R. “Overexpression of cyclin D1 mRNA in colorectal carcinomas and relationship to clinicopathological features: an in situ hybridization analysis” Pathol Oncol Res 6:65-70 (2000).
Laping, N. J., Olson, B. A., and Zhu, Y. “Identification of a novel nuclear guanosine triphosphate-binding protein differentially expressed in renal disease” J Am Soc Nephrol 12:883-890 (2001).
Manders, P., Bult, P., Sweep, C. G., Tjan-Heijnen, V. C., and Beex, L. V. “The prognostic value of the mitotic activity index in patients with primary breast cancer who were not treated with adjuvant systemic therapy” Breast Cancer Res Treat 77:77-84 (2003).
Makretsov, N. A., Huntsman, D. G., Nielsen, T. O., Yorida, E., Peacock, M., Cheang M. C., Dunn, S. E., Hayes, M., van de Rijn, M., Bajdik, C., et al. “Hierarchical clustering analysis of tissue microarray immunostaining data identifies prognostically significant groups of breast carcinoma” Clin Cancer Res 10:6143-6151 (2004).
Michels, J. J., Marnay, J., Delozier, T., Denoux, Y., and Chasle, J. “Proliferative activity in primary breast carcinomas is a salient prognostic factor” Cancer 100:455-464 (2004).
Miller C L, Yolken R H. “Methods to optimize the generation of cDNA from postmortem human brain tissue” Brain Res Brain Res Protoc 10:156-167 (2003).
Mischel P S, Nelson S F, Cloughesy T F. “Molecular analysis of glioblastoma: pathway profiling and its implications for patent therapy” Cancer Biol Ther 2:242-247 (2003).
Nielsen, T. O., Hsu, F. D., Jensen, K., Cheang, M., Karaca, G., Hu, Z., Hernandez-Boussard, T., Livasy, C., Cowan, D., Dressler, L., et al. “Immunohistochemical and clinical characterization of the basal-like subtype of invasive breast carcinoma” Clin Cancer Res 10:5367-5374 (2004).
Paik, S., Shak, S., Tang, G., Kim, C., Baker, J., Cronin, M., Baehner, F. L., Walker, M. G., Watson, D., Park, T., et al. “A multigene assay to predict recurrence of tamoxifen-treated, node-negative breast cancer” N Engl J Med 351:2817-2826 (2004).
Panaro N J, Yuen P K, Sakazume T, Fortina P, Kricka L T, Wilding P. “Evaluation of DNA fragment sizing and quantification by the agilent 2100 bioanalyzer” Clin Chem 46:1851-1853 (2000).
Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C, Zhu S X, Lonning P E, Borresen-Dale A L, Brown P O, Botstein D. “Molecular portraits of human breast tumours” Nature 406:747-752 (2000).
Perou C M, Brown P O, Botstein D. “Tumor classification using gene expression patterns from DNA microarrays” New Technologies for life sciences: A Trends Guide pp 67-76 (2000).
Perou, C M., Jeffrey, S. S., van de Rijn, M., Rees, C. A., Eisen, M. B., Ross, D. T., Pergamenschikov, A., Williams, C. F., Zhu, S. X., Lee, I C., et al. “Distinctive gene expression patterns in human mammary epithelial cells and breast cancers” Proc Natl Acad Sci USA 96:9212-9217 (1999).
Pinheiro J C B D. “Mixed-effects models in S and S-PLUS” New York, Springer (2000). Pollack, J. R., Sorlie, T., Perou, C. M., Rees, C. A., Jeffrey, S. S., Lonning, P. E., Tibshirani, R., Botstein, D., Borresen-Dale, A. L., and Brown, P. O. “Microarray analysis reveals a major direct role of DNA copy number alteration in the transcriptional program of human breast tumors” Proc Natl Acad Sci USA 99:12963-12968 (2002).
Pollack, J R., Perou, C. M., Alizadela, A. A., Eisen, M. B., Pergamenschikov, A., Williams, C. F., Jeffrey, S. S., Botstein, D., and Brown, P. O. “Genome-wide analysis of DNA copy-number changes using cDNA microarrays” Nature Genetics 23:41-46 (1999).
Rasmussen R P. “Quantification on the LightCycler. In Rapid Cycle Real-Time PCR: Methods and Applications” Edited by Wittwer C T, Meuer, S., Nakagawara, K. Heidelberg, Springer Verlag, pp 21-34 (2001).
Robbins, P., Pinder, S., de Klerk, N., Dawkins, H., Harvey, J., Sterrett, G., Ellis, I., and Elston, C. “Histological grading of breast carcinomas: a study of interobserver agreement” Hum Pathol 26:873-879 (1995).
Ross, D. T., Scherf, U., Eisen, M. B., Perou, C. M., Rees, C., Spellman, P., Iyer, V., Jeffrey, S. S., Van de Rijn, M., Waltham, M., et al. “Systematic variation in gene expression patterns in human cancer cell lines [see comments]” Nat Genet 24:227-235 (2000).
Roux S, Pichaud F, Quinn J, Lalande A, Morieux C, Jullienne A, de Vernejoul M C. “Effects of prostaglandins on human hematopoietic osteoclast precursors” Endocrinology 138:1476-1482 (1997).
SantaLucia J. “A unified view of polymer, dumbbell, and oligonucleotide DNA nearest-neighbor thermodynamics” Proc Natl Acad Sci USA 95:1460-1465 (1998).
Schena M, Shalon D, Davis R W, Brown P O. “Quantitative monitoring of gene expression patterns with a complementary DNA microarray” Science 270:467-470 (1995).
Schwarz G. “Estimating the dimension of a model” The Annals of Statistics 6:461-464 (1978).
Singletary, S. E., Allred, C., Ashley, P., Bassett, L. W., Berry, D., Bland, K. I., Borgen, P. I., Clark, G. M., Edge, S. B., Hayes, D. F., et al. “Staging system for breast cancer” revisions for the 6th edition of the AJCC Cancer Staging Manual. Surg Clin North Am 83:803-819 (2003).
Sørlie, T., Tibshirani, R., Parker, J., Hastie, T., Marron, J. S., Nobel, A., Deng, S., Johnsen, H., Pesich, R., Geisler, S., et al. “Repeated observation of breast tumor subtypes in independent gene expression data sets” Proc Natl Acad Sci USA 100:8418-8423 (2003).
Sørlie, T., Perou, C. M., Tibshirani, R., Aas, T., Geisler, S., Johnsen, H., Hastie, T., Eisen, M. B., van de Rijn, M., Jeffrey, S. S., et al. “Gene expression patterns of breast carcinomas distinguish tumor subclasses with clinical implications” Proc Natl Acad Sci USA 98:1086940874 (2001).
Sotiriou, C., Neo, S. Y., McShane, L. M., Korn, E L., Long, P. M., Jazaeri, A., Martiat, P., Fox, S. B., Harris, A. L., and Liu, E. T. “Breast cancer classification and prognosis based on gene expression profiles from a population-based study” Proc Natl. Acad Sci USA 100:10393-10398 (2003).
Spanakis E. “Problems related to the interpretation of autoradiographic data on gene expression using common constitutive transcripts as controls” Nucleic Acids Res 21:3809-3819 (1993).
Suzuki T, Higgins P J, Crawford D R. “Control selection for RNA quantitation” Biotechniques 29:332-337 (2000).
Szabo, A., Perou, C. M., Karaca, M., Perreard, L., Quackenbush, U., and Bernard, P. S. “Statistical modeling for selecting housekeeper genes” Genome Biol 5:R59 (2004).
Taylor-Papadimitriou, J., Stampfer, M., Bartek, J., Lewis, A., Boshell, M., Lane, E. B., and Leigh, I. M. “Keratin expression in human mammary epithelial cells cultured from normal and malignant tissue: relation to in vivo phenotypes and influence of medium” J Cell Sci 94:403413 (1989).
Troyanskaya, O., Cantor, M., Sherlock, G., Brown, P., Hastie, T., Tibshirani, R., Botstein, D., and Altman, R B. “Missing value estimation methods for DNA microarrays” Bioinformatics 17:520-525 (2001).
Tubbs R R, Pettay J D, Roche P C, Stoler M H, Jenkins R B, Grogan T M. “Discrepancies in clinical laboratory testing of eligibility for trastuzumab therapy: apparent immunohistochemical false-positives do not get the message” J Clin Oncol 19:2714-2721 (2001).
van de Vijver M J, He Y D, van't Veer Li, Dai H, Hart A A, Voskuil D W, Schreiber G I, Peterse J L, Roberts C, Marton M I, Parrish M, Atsma D, Witteveen A, Glas A, Delahaye L, van der Velde T, Bartelink H, Rodenhuis S, Rutgers E T, Friend S H, Bernards R. “A gene-expression signature as a predictor of survival in breast cancer” N Engl I Med 347:1999-2009 (2002).
van't Veer, L J., Dai, H., van de Vijver, M J., He, Y. D., Hart, A. A., Mao, M., Peterse, H. L., van der Kooy, K., Marton, M. J., Witteveen, A T., et al. “Gene expression profiling predicts clinical outcome of breast cancer” Nature 415:530-536 (2002).
Vandesompele I, De Preter K, Pattyn F, Poppe B, Van Roy N, De Paepe A, Speleman F. “Accurate normalization of real-time quantitative RT-PCR data by geometric averaging of multiple internal control genes” Genome Biol 3:RESEARCH0034 (2002).
Welsh J B, Zarrinkar P P, Sapinoso L M, Kern S G, Behling C A, Monk B J, Lockhart D I, Burger R A, Hampton G M. “Analysis of gene expression profiles in normal and neoplastic ovarian tissue samples identifies candidate molecular markers of epithelial ovarian cancer” Proc Natl Acad Sci USA 98:1176-1181 (2001).
West, M., Blanchette, C., Dressman, H., Huang, E., Ishida, S., Spang, R., Zuzan, H., Olson, J. A., Jr., Marks, J. R., and Nevins, J. R. “Predicting the clinical status of human breast cancer by using gene expression profiles” Proc Natl Acad Sci USA 98:11462-11467 (2001).
Whitfield, M. L., Sherlock, G., Saldanha, A. J., Murray, J. I., Ball, C. A., Alexander, K. E., Matese, J. C., Perou, C. M., Hurt, M. M., Brown, P. O., et al. “Identification of genes periodically expressed in the human cell cycle and their expression in tumors” Mol Biol Cell 13:1977-2000(2002).
Wittwer C T, a. K., N. “Real-time PCR. In Molecular Microbiology” T. Persing D H, F C, Versalovic, I, Tang, Y W, Unger, E R, Relman, D A, and White, V, editor. Washington, D.C.: ASM Press (2004).
Yang, Y. H., Dudoit, S., Luu, P., Lin, D. M., Peng, V., Ngai, I., and Speed, T. P. “Normalization for cDNA microarray data: a robust composite method addressing single and multiple slide systematic variation” Nucleic Acids Res 30:e15 (2002).
Yu, K., Lee, C. H., Tan, P. H., and Tan, P. “Conservation of breast cancer molecular subtypes and transcriptional patterns of tumor progression across distinct ethnic populations” Clin Cancer Res 10:5508-5517 (2004).

What is claimed is:

1. A method of diagnosing cancer, the method comprising comparing expression levels of a combination of genes from Table 21 to test nucleic acids, wherein specific expression patterns of the test nucleic acids indicates a cancerous state.

2. The method of claim 1, wherein the combination of genes includes at least 10 genes from Table 21.

3. The method of claim 1, wherein the combination of genes includes at least 25 genes from Table 21.

4. The method of claim 1, wherein the combination of genes includes at least 50 genes from Table 21.

5. The method of claim 1, wherein the combination of genes includes at least 75 genes from Table 21.

6. A method of quantitating level of expression of a test nucleic acid comprising: a) comparing gene expression levels of a combination of genes from Table 21 to test nucleic acids corresponding to the same combination of genes; and b) quantitating level of expression of the test nucleic acid.

7. A method determining prognosis based on expression patterns in a subject diagnosed with cancer comprising: a) comparing expression levels of a combination of genes from Table 21 to test nucleic acids corresponding to the same combination of genes, b) identifying a subtype of cancer of the subject, and c) determining prognosis based on expression patterns in the subject.

8. A method of classifying cancer in a subject, comprising: a) identifying intrinsic genes of the subject to be used to classify the cancer; b) obtaining a sample from the subject; c) amplifying and detecting levels of intrinsic genes in the subject; and d) classifying cancer based upon results of step c.

9. A method of diagnosing cancer in a subject the method comprising: a) amplifying and detecting intrinsic genes; and b) diagnosing cancer based on expression levels of the gene within the subject.

10. A method of deriving a minimal intrinsic gene set for making biological classifications of cancer comprising:
a) collecting data from multiple samples from the same individual to identify potential intrinsic classifier genes;
b) weighting intrinsic classifier genes of multiple individuals identified using the method of step a relative to each other and forming classification dusters;
c) estimating the number of clusters formed in step b) and assigning individual samples to classification clusters;
d) identifying genes that optimally distinguish the samples in the assigned groups of step c);
e) performing iterative cross-validation with a nearest centroid classifier and overlapping gene sets of various sizes using the genes identified in step d); and
f) choosing a gene set which provides the highest class prediction accuracy when compared to the classifications made in step b).

11. The method of claim 10, wherein the cancer is selected from the group consisting of breast cancer, colon cancer, or melanoma.

12. The method of claim 1, wherein the genes are derived from fresh samples.

13. The method of claim 1, wherein the genes are derived from formalin-fixed paraffin embedded (FFPE) samples.

14. The method of claim 10, wherein sample comprises mRNA.

15. The method of claim 10, wherein the sample is amplified by PCR.

16. The method of claim 15, wherein the PCR is real time PCR.

17. The method of claim 11, wherein the breast cancer is classified into luminal, normal-like, HER2+/ER−, and basal-like.

18. The method of claim 10, wherein the intrinsic gene set is identified using a microarray.

19. The method of claim 10, wherein the intrinsic gene set is modified from a microarray.

20. The method of claim 19, wherein the intrinsic gene set includes at least one housekeeper gene.

21. A method of assigning a sample to an intrinsic subtype, comprising:
a) creating an intrinsic subtype average profile (centroid) for each subtype;
b) individually comparing a new sample to each centroid; and
c) assigning the new sample to the centroid that is most similar to an expression profile of the new sample.
